(19) United States
(12) Patent Application Publication
(10) Pub. No.: US 2004/0049352 A1
(43) Pub. Date: Mar. 11, 2004

(54) Designing modulators for glycosyltransferases

(76) Inventors: Isabelle Andre, Toronto (CA); Igor Tvoroska, Toronto (CA); Mohan Rao, Toronto (CA); Tibor Kozar, Toronto (CA)

Correspondence Address: MERCHANT & GOULD PC, P.O. BOX 2903, MINNEAPOLIS, MN, 55402-0903, US

(21) Appl. No.: 10/275,572

(22) PCT Filed: May 10, 2001
(86) PCT No.: PCT/CA01/00656

(30) Foreign Application Priority Data
May 10, 2000 (US) 60203316

(51) Int. Cl. G06F 19/00; G01N 33/48; G01N 33/50; C12N 9/10

(52) U.S. Cl. 702/19; 435/193

(57) ABSTRACT

The invention relates to structures and models of glycosyltransferases and ligand binding domains of glycosyltransferases, and complexes of the glycosyltransferases and ligand binding domains with ligands. The structural coordinates that define the structures and models enable the determination of homologues, the structures of polypeptides with unknown structure, and the identification of modulators of the glycosyltransferases. The invention also relates to structures and models of nucleotide-sugar donors and acceptors for the glycosyltransferases, and the design of modulators for the glycosyltransferases based on the properties of these structures and models.

### FIELD OF THE INVENTION

The invention relates to structures and models of glycosyltransferases and ligand binding domains of glycosyltransferases, and complexes of the glycosyltransferases and ligand binding domains with ligands. The structural coordinates that define the structures and models enable the determination of homologues, the structures of polypeptides with unknown structure, and the identification of modulators of the glycosyltransferases. The invention also relates to structures and models of nucleotide-sugar donors and acceptors for the glycosyltransferases, and the design of modulators for the glycosyltransferases based on the properties of these structures and models.

### BACKGROUND OF THE INVENTION

Glycosyltransferases (GTs, a general nomenclature for glycosyltransferases is EC 2.4.x.y) comprise a group of enzymes that are involved in the biosynthesis of complex oligosaccharides (1-4). The result of the reaction catalyzed by these enzymes is the formation of a new glycosidic linkage and it appears that there is at least one distinct glycosyltransferase for every type of glycosidic linkage. Glycosylation proceeds in a stepwise manner and, therefore, the expression and specificity of the enzymes represent key regulatory factors in defining the repertoire of biosynthesized oligosaccharides. During oligosaccharide processing, oligosaccharides are converted into hybrid and complex oligosaccharides by addition of N-acetylglucosaminyl residues (GlcNAc, 2-acetamido-2-deoxy-α-D-glucopyranosyl). These modifications in the oligosaccharide chains of N- and O-linked glycoproteins accompany many physiological and pathological cell processes (5). The transfer of GlcNAc is catalyzed by N-acetylglucosaminyltransferases (GlcNAc-Ts or GnTs). In such a transfer, the donor of the GlcNAc residue is UDP-GlcNAc [uridine 5′-(2-acetamido-2-deoxy-α-D-glucopyranosyl pyrophosphate)] while the acceptor is one of the hydroxyl groups located at a particular position of a variety of oligosaccharides. N-acetylglucosaminyltransferases show a decisive specificity for the oligosaccharide-acceptor and they generally require the presence of a metal cofactor (6). There are at least eight different GlcNAc-Ts involved in the biosynthesis of complex and hybrid N-glycans (GlcNAc-T I-GlcNAc-T VIII), five in the biosynthesis of O-glycans (Core 2 - Core 4 GnTs, Core 1 and Core 2 elongation GnTs), and two in the biosynthesis of antigen determinants (blood group i and blood group I) (1, 2,4,7). Though some of these GlcNAc-Ts have already been cloned, the origin of their specificity remains unknown due to the lack of experimental structures of GlcNAc-Ts or any other mammalian glycosyltransferase.

In spite of their large abundance in nature, crystal structures of glycosyltransferases are rare. Until recently, the only structure of glycosyltransferase available was that of a DNA-modifying β-glucosyltransferase from bacteriophage T4 and its complex with UDP-Glc (8). However, that enzyme is somewhat different from other glycosyltransferases since the acceptor involved in the reaction with this enzyme is not a carbohydrate. Indeed, this enzyme catalyses the transfer of a glucose moiety from UDP-glucose to hydroxymethylated cytosines of DNA. The DNA-modifying β-glucosyltransferase from bacteriophage T4 presents no sequence homology to any other glycosyltransferase (9) though the structure of this enzyme has been used as a template to predict the structure of other glycosyltransferases (10). Recently, a decisive breakthrough in this field has been achieved with the resolution of the X-ray structures of two bacterial glycosyltransferases in their native and nucleotide-complexed forms, the SpsA (11), for which the substrate specificity is undefined, and the β1,4-galactosyltransferase T1 (12).

The reaction catalyzed by GlcNAc-Ts can be regarded as a nucleophilic displacement of the UDP (uridine 5′-pyrophosphate) functional group at the anomeric carbon C₁ of the GlcNAc (2-acetamido-2-deoxy-α-D-glucopyranose) residue of UDP-GlcNAc by a hydroxyl group of a specific oligosaccharide-acceptor (FIG. 36). The enzymatic reaction of all known GlcNAc-Ts, except the α-1,4-GlcNAc-T (13), leads to an inversion of the anomeric configuration. There is a clear resemblance between the enzymatic action of glycosyltransferases and the enzymatic action of glycoside hydrolases, mechanisms of which have largely been characterized in detail (14-21). Many aspects of the functions and catalytic mechanisms of N-acetylglucosaminyltransferases are, however, still unknown since only few mechanistic studies on N-acetylglucosaminyltransferases have been reported to date (6,22). In the absence of experimental data, high-level ab initio calculations can be used to gain some insight into many characteristics of the enzymatic reaction catalyzed by N-acetylglucosaminyltransferases. They can provide the description on an atomic level of the discrete intermediates and transition states found along the enzyme-catalyzed reaction pathway.

### SUMMARY OF THE INVENTION

The applicants have produced high-level ab initio quantum chemical results on a model of the GlcNAc transfer reaction catalyzed by N-acetylglucosaminyltransferases and, based on the results additionally developed homology models for glycosyltransferases and ligand binding domains thereof, and complexes of the enzymes or ligand binding domains with ligands including sugar nucleotide donors and acceptors. In particular, applicants have produced models and structures for GnT1, GnTV, core 2L, core 2b/M, and core 3, ligand binding domains thereof, and complexes of the enzymes, for example with UDP, UDP-GlcNAc and acceptors. Models and structures have also been produced for transition states of GnT1 and core 2L.

Therefore, the invention provides a model or secondary, tertiary, and/or quantemary structure of a ligand binding domain of a glycosyltransferase. Binding domains are of significant utility in drug discovery. The association of natural ligands and substrates with the ligand binding domains of glycosyltransferases is the basis of biological mechanisms. The associations may occur with all or any parts of a ligand binding domain. An understanding of these associations is the basis for the design and optimization of drugs having more favorable associations with their target enzyme and thus provide improved biological effects. Therefore, information about the shape and structure of glycosyltransferases and their ligand-binding domains is invaluable in designing potential modulators of glycosyltransferases for use in treating diseases and conditions associated with or modulated by the glycosyltransferases.

Ligand binding domains include one or more of the binding domains for a disphosphate group or pyrophosphate of a sugar nucleotide donor, a nucleotide of a sugar nucleotide donor, a nitrogeneous heterocyclic base (preferably a pyrimidine base, more preferably uracil) of a sugar nucleotide donor, a sugar of the nucleotide of a sugar nucleotide donor, a selected sugar of a sugar nucleotide donor that is transferred to an acceptor, and/or an acceptor. The structure of a ligand binding domain may be defined by selected binding sites or atomic interactions in the domain.

In accordance with aspects of the invention, a ligand binding domain is defined by (a) one or more (preferably all) amino acid residues of a GnT1 shown in Table 10; (b) one or more (preferably all) amino acid residues of a GnTV shown in Table 11; (c) one or more (preferably all) amino acid residues of a core 2L/T1 shown in Table 12; and (d) one or more (preferably all) amino acid residues of a core 2b/2M/T2 shown in Table 13. The invention also relates to a model or secondary, tertiary, and/or quantemary structure of a ligand binding domain of a glycosyltransferase defined by the structural coordinates of one or more of the atomic contacts or atomic interactions as shown in Table 10, Table 11, Table 12, or Table 13. Each of the atomic interactions is defined in Table 10, 11, 12, or 13 by an atomic contact (more preferably a specific atom where indicated) on the sugar nucleotide donor or part thereof, and an atomic contact (more preferably a specific atom where indicated) on the glycosyltransferase.

The invention also provides a model of a ligand binding domain designed in accordance with a method of the invention.

The invention further provides a model or secondary, tertiary and/or quantemary structure of a glycosyltransferase or a transition state of a glycosyltransferase.

The invention contemplates a model or secondary, tertiary and/or quanternary structure of a glycosyltransferase or ligand binding domain in association with a ligand or substrate.

The structures and models of the invention provide information about die atomic contacts involved in the interaction between the enzyme and a known ligand which can be used to screen for unknown ligands. Therefore the present invention provides a method of screening for a ligand capable of binding a glycosyltransferase ligand binding domain, comprising the use of a secondary, tertiary or quantemary structure or a model of the invention. For example, the method may comprise the step of contacting a ligand binding domain with a test compound, and determining if the test compound binds to the ligand.

A structure or model of the invention may be used to design, evaluate, and identify ligands of glycosyltransferase other than ligands that associate with a glycosyltransferase. The ligands may be based on the shape and structure of a glycosyltransferase, or a ligand binding domain or atomic interactions, or atomic contacts thereof. Therefore, ligands, in particular modulators, may be derived from ligand binding domains or analogues or parts thereof.

The present invention also contemplates a ligand identified by a method of the invention. A ligand may be a competitive or non-competitive inhibitor of a glycosyltransferase. Preferably, the ligand is a modulator that is capable of modulating the activity of a glycosyltransferase enzyme. Thus, the methods of the invention permit the identification early in the drug development cycle of compounds that have advantageous properties.

In an embodiment, the present invention contemplates a method of identifying a modulator of a glycosyltransferase or a ligand binding domain or binding site thereof, comprising the step of using the structural coordinates of a glycosyltransferase or a ligand binding domain or binding site thereof, or a model of the invention to computationally evaluate a test compound for its ability to associate with the glycosyltransferase or ligand binding domain or binding site thereof. Use of the structural coordinates of a glycosyltransferase structure, ligand binding domain, or binding site thereof, of the invention to identify a ligand or modulator is also provided.

In another embodiment of the invention, a method is provided for identifying a potential modulator of a glycosyltransferase by determining binding interactions between a test compound and atomic contacts of a ligand binding domain of a glycosyltransferase defined in accordance with the invention comprising:

(a) generating the atomic contacts on a computer screen;

(b) generating test compounds with their spatial structure on the computer screen; and

(c) determining whether the compounds associate or interact with the atomic contacts defining the glycosyltransferase;

(d) identifying test compounds that are potential modulators by their ability to enter into a selected number of atomic contacts.

Another aspect of the invention provides methods for identifying a potential modulator of a glycosyltransferase function by docking a computer representation of a test compound with a computer representation of a structure of a glycosyltransferase or a ligand binding domain thereof that is defined as described herein. In an embodiment the method comprises the following steps:

(a) docking a computer representation of a compound from a computer data base with a computer representation of atomic interactions or atomic contacts of a ligand binding domain of a glycosyltransferase to obtain a complex;

(b) determining a conformation of the complex with a favourable geometric fit and favourable complementary interactions; and

(c) identifying test compounds that best fit the atomic interactions or contacts as potential modulators of the glycosyltransferase.

In another embodiment the method comprises the following steps:

(a) modifying a computer representation of a test compound complexed with a ligand binding domain of a glycosyltransferase by deleting or adding a chemical group or groups;

(b) determining a conformation of the complex with a favourable geometric fit and favourable complementary interactions; and

(c) identifying a test compound that best fits the ligand binding domain as a potential modulator of a glycosyltransferase.

In still another embodiment the method comprises the following steps:

(a) selecting a computer representation of a test compound complexed with atomic contacts or atomic interactions of a binding domain of a glycosyltransferase; and

(b) searching for molecules in a data base that are similar to the test compound using a searching computer program, or replacing portions of the test compound with similar chemical structures from a data base using a compound building computer program.

The ligands or compounds identified according to the methods of the invention preferably have structures such that they are able to enter into an association with a ligand binding domain. Selected ligands or compounds may be characterized by their suitability for binding to particular ligand binding domains. A ligand binding domain or binding site may be regarded as a type of negative template with which the compounds correlate as positives in the manner described herein and thus the compounds are unambiguously defined. Therefore, it is possible to describe the structure of a compound suitable as a modulator of a glycosyltransferase by accurately defining the atomic interactions to which the compound binds to a ligand binding domain and deriving the structure of the compound from the spacial structure of the target.

The invention contemplates a method for the design of ligands, in particular modulators, for glycosyltransferase based on the secondary, tertiary or quanternary structure of a sugar nucleotide donor (or part thereof) defined in relation to its spatial association with the three dimensional structure of the glycosyltransferase or a ligand binding domain thereof. Generally, a method is provided for designing potential inhibitors of a glycosyltransferase comprising the step of using the structural coordinates of a sugar nucleotide donor or part thereof, defined in relation to its spatial association with the secondary, tertiary or quantemary structure or model of a glycosyltransferase or a ligand binding domain thereof, to generate a compound for associating with the ligand binding domain of the glycosyltransferase. The following steps are employed in a particular method of the invention: (a) generating a computer representation of a sugar nucleotide donor, or part thereof, defined in relation to its spatial association with the three dimensional Structure of a glycosyltransferase or a ligand binding domain thereof, (b) searching for molecules in a data base that are similar to the defined sugar nucleotide donor, or part thereof, using a searching computer program, or replacing portions of the compound with similar chemical structures from a database using a compound building computer program.

Therefore, the invention further contemplates classes of ligands, in particular modulators, of a glycosyltransferase based on the secondary, tertiary or quantemary structure of a sugar nucleotide donor, or part thereof, defined in relation to the sugar nucleotide donor's spatial association with a three dimensional structure of a glycosyltransferase.

It will be appreciated that a ligand or modulator of a glycosyltransferase may be identified by generating an actual secondary or three-dimensional model of a ligand binding domain or binding site, synthesizing a compound, and examining the components to find whether the required interaction occurs.

Modulators which are capable of modulating the activity of glycosyltransferases have therapeutic and prophylactic potential. Therefore, the methods of the invention for identifying modulators may comprise one or more of the following additional steps:

(a) testing whether the ligand is a modulator of the activity of a glycosyltransferase, preferably testing the activity of the modulator in cellular assays and animal model assays;

(b) modifying the modulator;

(c) optionally rerunning steps (a) or (b); and

(d) preparing a pharmaceutical composition comprising the modulator.

Steps (a), (b) (c) and (d) may be carried out in any order, at different points in time, and they need not be sequential.

Still another aspect of the invention provides a method of conducting a drug discovery business comprising:

(a) providing one or more systems or methods for identifying modulators based on a model or structure of the present invention, preferably a method using a computer as described herein;

(b) conducting therapeutic profiling of modulators identified in step (a), or further analogs thereof, for efficacy and toxicity in animals; and

(c) formulating a pharmaceutical composition including one or more agents identified in step (b) as having an acceptable therapeutic profile.

In certain embodiments, the subject method may also include a step of establishing a distribution system for distributing the pharmaceutical composition for sale, and may optionally include establishing a sales group for marketing the pharmaceutical composition.

In yet another aspect of the invention, a method of conducting a target discovery business is provided comprising:

(a) providing one or more system or method for identifying modulators based on a model or structure of the present invention, preferably a method using a computer as described herein;

(b) optionally conducting therapeutic profiling of modulators identified in (a) for efficacy and toxicity in animals; and

(c) licensing to a third party the rights for further drug development and/or sales for agents identified in step (a), or analogs thereof.

There is also provided a pharmaceutical composition comprising a modulator, and a method of treating and/or preventing disease associated with a glycosyltransferase comprising the step of administering a modulator or pharmaceutical composition comprising a modulator to a patient.

In an aspect, the invention contemplates a method of treating a disease associated with a glycosyltransferase with inappropriate activity in a cellular organism, comprising:

(a) administering a modulator identified using the methods of the invention in an acceptable pharmaceutical preparation; and

(b) activating or inhibiting a glycosyltransferase to treat the disease.

The invention provides for the use of a modulator identified by the methods of the invention in the preparation of a medicament to treat a disease associated with a glycosyltransferase with inappropriate activity in a cellular organism. Use of the structural coordinates of a glycosyltransferase structure of the invention to manufacture a medicament is also provided.

Another aspect of the invention provides machine readable media encoded with data representing a model of the invention or the coordinates of a structure of a glycosyltransferase or ligand binding domain or binding site thereof as defined herein, or the three dimensional structure of a sugar nucleotide donor or part thereof defined in relation to its spatial association with a three dimensional structure of a glycosyltransferase as defined herein. The invention also provides computerized representations of a model of the invention or the secondary, tertiary or quanternary structures of the invention, including any electronic, magnetic, or electromagnetic storage forms of the data needed to define the structures such that the data will be computer readable for purposes of display and/or manipulation. The invention further provides a computer programmed with a homology model of a ligand binding domain of a glycosyltransferase. The invention still further contemplates the use of a homology model of the invention as input to a computer programmed for drug design and/or database searching and/or molecular graphic imaging in order to identify new ligands or modulators for glycosyltransferases.

These and other aspects of the present invention will become evident upon reference to the following detailed description and attached drawings.

### BRIEF DESCRIPTION OF THE DRAWINGS

The invention will now be described in relation to the drawings in which:

FIG. 1. (a) Potential Energy Surface calculated at the HF/6-31G* level and corresponding to the mechanism involving only a catalytic base to assist the nucleophilic attack followed by the proton transfer to the base (3-A); (b) Geometrical representation of the different stationary points calculated at the DFT/B3LYP/6-31G* level. Numbers in italic represent relative energies (in kcal/mol) at DFT/B3LYP/6-31++G**//DFT/B3LYP/6-31G* level. R, TS, INT, and PC represent the reactants, transition states, intermediates, and products, respectively.

FIG. 2. (a) Potential Energy Surface calculated at the HF/6-31G* level and corresponding to the mechanism involving a pair of catalytic amino acids to assist the proton transfer to O1 and the nucleophilic attack. In this mechanism, the proton Ha is located at the acceptor (Scheme 3-B). (b) Geometrical representation of the different stationary points calculated at the DFT/B3LYP/6-31G* level. Numbers in italic represent relative energies (in kcal/mol) at DFT/B3LYP/6-31++G**//DFT/B3LYP/6-31G* level. R, TS, INT, and PC represent the reactants, transition states, intermediates, and products, respectively.

FIG. 3. (a) Potential Energy Surface calculated at the HF/6-31G* level and corresponding to the mechanism involving a pair of catalytic amino acids to assist the proton transfer to O1 and the nucleophilic attack. In this mechanism, the proton Ha is positioned at the base (Scheme 3-B). (b) Geometrical representation of the different stationary points calculated at the DFT/B3LYP/6-31G* level. Numbers in italic represent relative energies (in kcal/mol) at DFT/B3LYP/6-31++G**//DFT/B3LYP/6-31G* level. R, TS, INT, and PC represent the reactants, transition states, intermediates, and products, respectively.

FIG. 4. (a) Crystal structures of SpsA and GnT I. (b) Superimposition of SpsA and GnT I structures.

FIG. 5. Sequence alignment of GnTI and Core 2L Gn T. Relevant amino acid residues of the GnT I binding site are highlighted.

FIG. 6. Homology models of Core 2L (left), Core 2b/M GnT (middle), and GnTV (right) based on the structure of GnT I.

FIG. 7. Representation of the electrostatic potential surface of GnT I and the favored binding modes of UDP docked within the enzyme.

FIG. 8. Representation of UDP binding interactions in the experimental structures of GnTs.

FIG. 9. Representation of the four top scoring UDP-Core2L GnT complexes. Amino acids interacting with the uridine part as described herein are shown in tube.

FIG. 10. Representation of the predicted lowest energy docking mode of the acceptor heptasaccharide into GnT I-UDPGlcNAc complex model. (a) enlarged view of the GlcNAc binding site. (b) overall view of the Transition state-GnT I complex.

FIG. 11. Predicted binding mode of the transition state in the Core2L GnT model.

FIG. 12. Representation of the electrostatic potential surface of the Core2L GnT model and UDP in its prominent binding mode. GlcNAc acceptor (GalNAc-Gal) binding regions are outlined in green.

FIG. 13. GnT V model complexed with the transition state of UDP-GlcNAc and the acceptor oligosaccharide.

FIG. 14. Predicted binding mode of GD500 in the model of Core2L GnT.

FIG. 15. Predicted binding modes for a fragment of GD0541 (in yellow) and an analogue where the GD0541 fragment is attached to Tacrine (in white).

FIG. 16. (a) Structure of Tetrahydroaminoacaridine (Tacrine). (b) Structure of a potential GD0541 analogue having the so-called Tacrine molecule attached to a fragment of GD0541 through an etheric linkage.

FIG. 17: A view showing a superimposition of GT's. The Figure shows the superimposition of the main chain atoms of GnT I (red), Core 2L GnT (green), SpsA (magenta), α-1,3-Ga1T (cyan) and GnT V (black).

FIG. 18a-d: The UDP recognition domain of a) Core 2L GnT; b) GalT; c) SpsA (Davies and co-workers); and d) GnT I (Rini and co-workers).

FIG. 18e: Overlay of the trace of the UDP recognition domain of GnT I and SpsA. The binding conformation of UDP is shown in tubes.

FIG. 19A shows the formula of the heptasaccharide acceptor for GnTV, which provides the basis for a potential modulator based on the acceptor for GnT V. The reactive groups in the molecule can be substituted with the list of groups set out elsewhere in the application.

FIG. 19B shows the formula of (1,6)-linked N-acetylglucosylamine linked to the heptasaccharide acceptor for GnT V. This is the product after the reaction with the enzyme.

FIG. 20 represents the schematic view of the resulting homology model of GnT V (right). For comparison the scheme of the GnT I template (left) is also shown. The overall shape of the binding pocket of GnT V in the center of the enzyme resembles the binding pocket of GnT I and as a result the docking of UDPGlcNAc is assumed to be similar.

FIG. 21 shows UDPGlcNAc in the active site of GnT V.

FIG. 22 shows UDP in the active site of GnT V.

FIG. 23 illustrates the orientation of the ligand in the binding pocket of GnT V complexed with UDPGlcNAc (top view). The uridine part of the molecule is stabilized (localized at the bottom part of the pocket) with hydrogen bonds and stacking interactions.

FIG. 24 shows the amino acids involved in the interactions with the UDPGlcNAc ligand. There are two low energy conformations presented from the top-ranking clusters of UDPGlcNAc. The Figure also illustrates the flexibility around the diphosphate linkages.

FIG. 25a shows the active site residues of the GnT I-UDP complex.

FIG. 25b shows a superimposition of the Core 2L GnT model (green) and the GnT I structure (red). The active site residues of Core 2L GnT are shown in tubes. The core region contains many of the common alpha helix and beta strand elements, including the active site residues Asp99 (Core 2L)/Asp144(GnT I), His 131 (Core 2L)/His190 (GnT I), Ile133 (Core 2L)/Ile187(GnT I), Glu159 (Core 2L)/Asp213 (GnT I). It is clear that the active site architectures and the residues that constitute the active site of Core 2L GnT and GnT I are highly conserved.

FIG. 26 shows the computed low energy docking modes of UDP to Core 2L GnT. The lowest energy-binding mode is shown as a thick tube. In all the top ranking binding modes of UDP shown in this Figure, the uridine group assumes a similar binding conformation.

FIG. 27 shows the lowest energy-docking mode of UDP on the solvent-excluded surface of the Core 2L GnT. The potential residues that interact with the uridine ring are shown in blue colored surface. The ribose ring and pyrophosphate groups of UDP are covered by the loop structure of Core 2L GnT.

FIG. 28 shows a view of the lowest energy-binding mode of UDP-GlcNAc to the Core 2L GnT. The Core 2L GnT is shown in a solvent excluded surface representation and the UDP-GlcNAc is shown in tubes.

FIG. 29 shows a close-up view of the sugar binding pocket. The sugar group of the UDP-GlcNAc occupies a site that is close to the hydrophobic region.

FIG. 30 shows an overall view of GlcNAc binding to the transition state of Core 2L GnT showing the hydrophobic pocket.

FIG. 31 shows a view of GlcNAc binding to the transition state of Core 2L GnT showing the hydrophobic pocket.

FIG. 32 shows the binding of the pyrophosphate of UDP-GlcNAc to the loop structure of Core 2L GnT.

FIG. 33 shows a GnT I acceptor.

FIG. 34 is a schematic energetic representation (in kcal/mol) of the possible reaction pathways observed in the different PESs for the transfer of GlcNAc catalyzed by inverting N-acetylglucosaminyltransferases. Relative energies are calculated at DFT/B3LYP/6-31++G**//DFT/B3LYP/6-31 G* level.

FIG. 35 is a geometrical representation of the transition states TS1-TS11 calculated at the DFT/B3LYP/6-31G* level. Transition states are clustered by similarities in their C1-Oa and C1-O1 bond lengths. Average C1-Oa and C1-O1 distances, calculated for each group, are noted on the figure. (A) TS2, TS5 and TS8 structures exhibit short C1-Oa (1.4-1.6 Å) and long C1-O1 bond lengths (2.8-3.2 Å). TS11 has been omitted from the structure superimposition for clarity purpose. (B) TS3, TS4 and TS9 structures display long C1-Oa (2.4-2.7 Å) and short C1-O1 (1.5-2.1 Å) bond lengths. TS10 has been omitted from the structure superimposition for clarity. (C) TS1, TS6 and TS7 structures exhibit elongated C1-Oa (2.1-2.4 Å) and C1-O1 (2.5-2.7 Å) bond lengths.

FIG. 36 is a schematic representation of the N-acetylglucosaminyltransferases involved in the biosynthesis of N-glycans (GlcNAc-T I-VIII), O-glycans (Core 2-4 and Core 1-2 elongation GnTs) and antigen determinants (blood groups i and I).

FIG. 37 is a schematic representation of the structural model used to describe the GlcNAc transfer by inverting N-acetylglucosaminyltransferases.

FIG. 38 is a schematic representation of the two different types of mechanism investigated for the transfer of GlcNAc by inverting N-acetylglucosaminyltransferases. Mechanism A involves only a catalytic base while two catalytic amino acids are implicated in mechanism B.

### DESCRIPTION OF THE TABLES

Table 1—Structural coordinates for GnT1.

Table 2—Structural coordinates for GnTV.

Table 3—Structural coordinates for core 2L or T1 (human).

Table 4—Structural coordinates for core 2L or T1(mouse)

Table 5—Structural coordinates for core 2L (bovine)

Table 6—Structural coordinates for core 2b/core M/core 2T2.

Table 7—Structural coordinates for core 2C (human)

Table 8—Structural coordinates for core 3.

Table 9—consensus polar and hydrophobic interactions in the UDP binding sites of GT-UDP complexes (the first columns are uracil atoms).

Table 10—Atomic interactions between a GnT1 and a nucleotide sugar donor.

Table 11—Atomic interactions between a GnTV and a nucleotide sugar donor.

Table 12—Atomic interactions between a core 2L or T1 and a nucleotide sugar donor.

Table 13—Atomic interactions between a core 2B and a nucleotide sugar donor.

Table 14—Structural coordinates for conformations of UDP in association with a GNTI/ground state.

Table 15—Structural coordinates for conformations of UDP in association with GnTV.

Table 16—Structural coordinates for conformations of UDP in association with core 2L.

Table 17—Structural coordinates for conformations of UDPGlcNAc in association with a GnT1 transition state.

Table 18—Structural coordinates for conformations of UDPGlcNAc in association with GnTV.

Table 19—Structural coordinates for conformations of UDPGlcNAc in association with a core 2L/transition state.

Table 20—Structural coordinates for conformations of an oligosaccharide acceptor in association with a GNT1.

Table 21—Structural coordinates for the loop structure for a GnT1.

Table 22—Structural coordinates for the loop structure for a Core 2L.

Table 23—Structural coordinates for the loop structure for a GnTV.

Table 24 is a list of N-acetylglucosylaminotransferases, and their sugar nucleotide donors and acceptors.

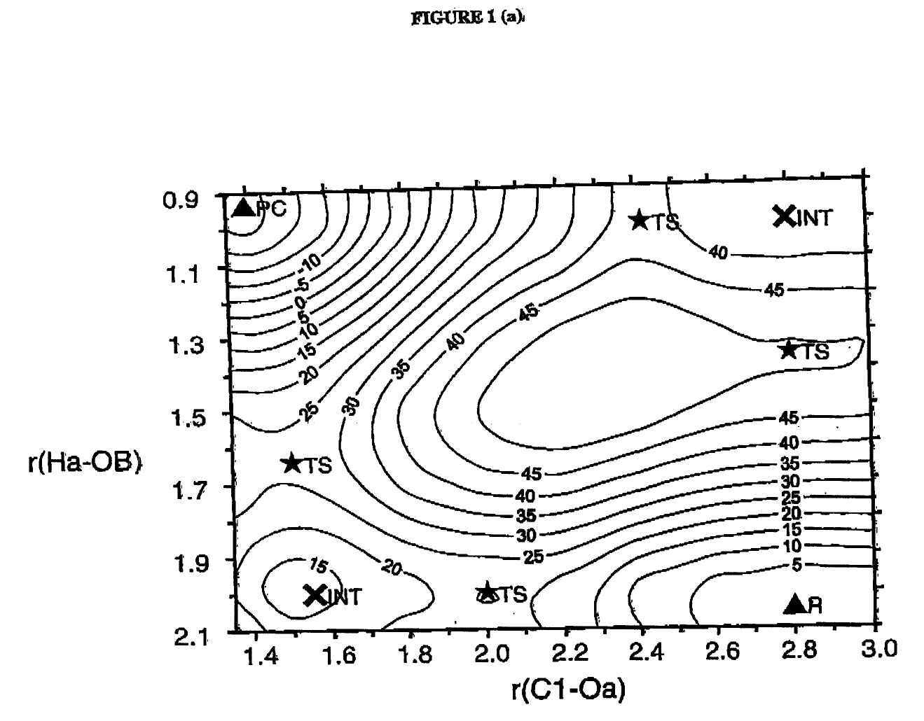

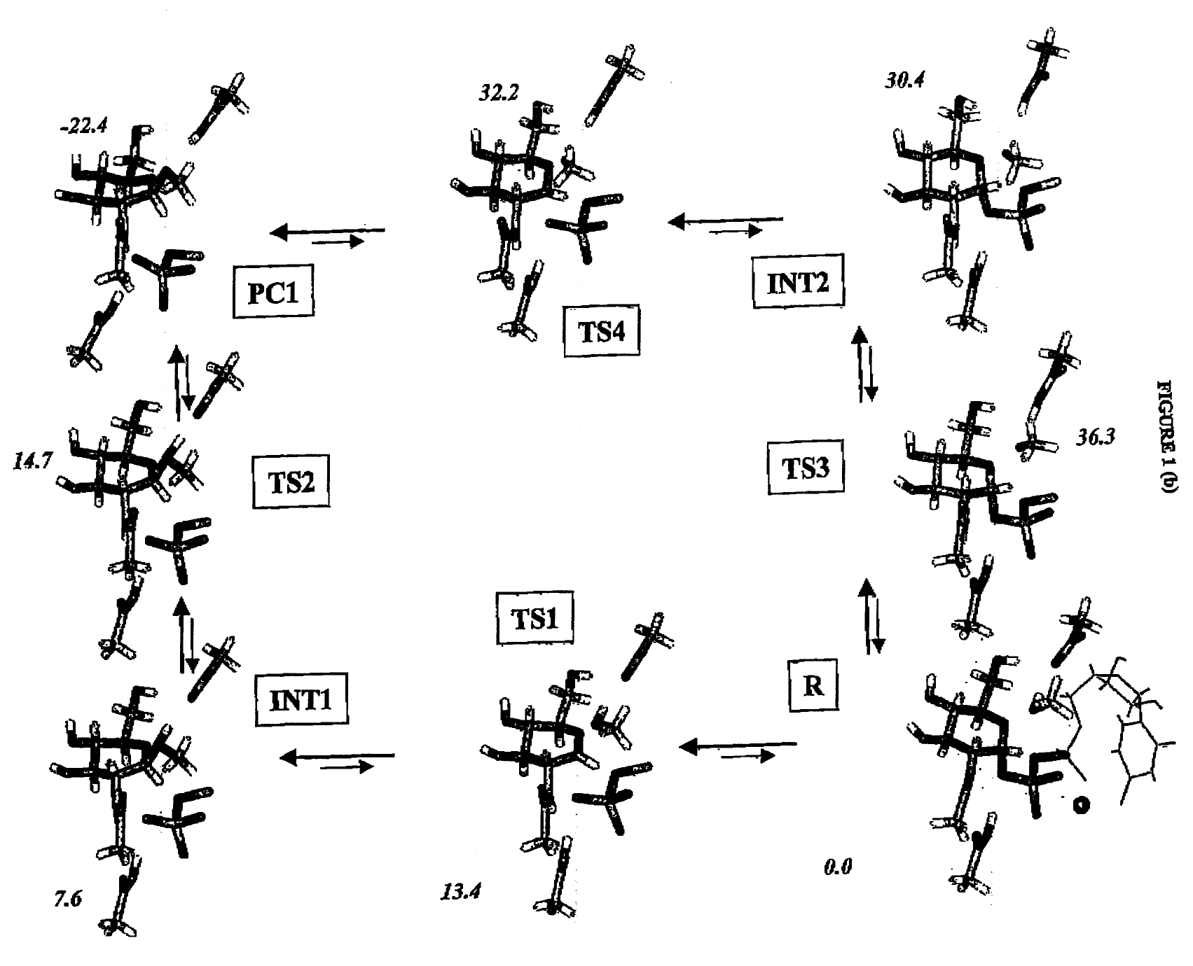

Table 25—Ab initio calculated Geometrical Parameters of the points observed on PESs described in FIGS. 1-3 at the HFG/6-31G* and DFT/B3YLYP/6-3G* levels.

Table 26—Comparison of the ab initio relative energies (kcal/mol) calculated by various methods for the points observed on PESs described on FIGS. 1-3.

In Tables 2 through 9 inclusive, from the left, the second column identifies the atom number; the third identifies the atom type; the fourth identifies the amino acid type; the fifth identifies the residue number; the sixth identifies the x coordinates; the seventh identifies the y coordinates; and the eighth identifies the z coordinates.

### DETAILED DESCRIPTION OF PREFERRED EMBODIMENTS

Definitions:

Unless otherwise indicated, all terms used herein have the same meaning as they would to one skilled in the art of the present invention. Practitioners are particularly directed to Current Protocols in Molecular Biology (Ansubel) for definitions and terms of the art. Abbreviations for amino acid residues are the standard 3-letter and/or 1-letter codes used in the art to refer to one of the 20 common L-amino acids.

The term “associate”, “association” or “associating” refers to a condition of proximity between a ligand, chemical entity, or compound, or portions or fragments thereof, and a glycosyltransferase, or portions or fragments thereof (e.g. ligand binding domain). The association may be non-covalent i.e. where the juxtaposition is energetically favored by for example, hydrogen-bonding, van der Waals, or electrostatic or hydrophobic interactions, or it may be covalent.

The term “glycosyltransferase” refers to an enzyme that catalyzes the transfer of a single monosaccharide unit from a donor to the hydroxyl group of an acceptor substrate. The acceptor can be either a free saccharide, glycoprotein, glycolipid, or polysaccharide. The donor can be a nucleotide-sugar, preferably UDP-GlcNAc. Glycosyltransferases show a precise specificity for both the sugar acceptor and donor and generally require the presence of a metal cofactor. Glycosyltransferases include but are not limited to eukaryotic glycosyltransferases involved in the biosynthesis of glycoproteins, glycolipids, glycosylphosphatidylinositols and other complex glycoconjugates, and prokaryotic glycosyltransferases involved in the synthesis of carbohydrate structures of bacteria and viruses, such as enzymes involved in LOS and lipopolysaccharide biosynthesis.

Glycosyltransferases are derivable from a variety of sources, including viruses, bacteria, fungi, plants, and animals. In a preferred embodiment the glycosyltransferases are derivable from an animal, preferably a mammal including but not limited to bovine, ovine, porcine, murine equine, most preferably a human. The enzyme may be from any source, whether natural, synthetic, semi-synthetic, or recombinant

Examples of glycosyltransferases are N-acetylglucosaminyltransferases, including N-acetylglucosaminyltransferases I through VIII (“GnT1” through “GnTVIII”) involved in the biosynthesis of complex and hybrid N-glycans; UDP-N-acetylglucosamine: N-acetyl galactosamine 1,6-N-acetylgucosaminyl transferases (core 2 GlcNAc transferases), Core 3 GlcNAc transferase, Core 4 GlcNAc transferase, and Core 1 and Core 2 elongation GnTs involved in the biosynthesis of 0-glycans, and the GnTs involved in the biosynthesis of antigen determinants (blood group i and blood group I) (Schachter, H. Curr. Opin. Struct. Biol. 1991, 1, 755-765; Montreuil, J.; Vliegenthart, J. F. G.; Schachter, H. Glycoproteiins; Neuberger, A. and van Deenen, L. L. M., Ed.; Elsevier. Amsterdam, 1995; Vol. 29a; and Raju, T. S.; Stanley, P. JBiol. Chem. 1998, 273, 14090-14098). Table 24 provides examples of eukaryotic glycosyltransferases, and their sugar nucleotide donors, and acceptors.

A number of core 2 GlcNAc transferases have been identified and cloned: Core 2 GnT1 (Core 2 GnT, Core 2 GnT-L, Core 2L/T1); Core 2 GnT2 (Core 2/4 GnT, Core 2 GnT-M, Core 2b/T2/M); and Core 2 GnT3 (Core 2c/T3) (Bierhuizen, 1. and Fukuda, M. 1999, Proc. Natl. Acad, Sci. U.S.A. 89, 9326-9330; Schwientek, T. et al, 1999, J. Biol. Chem. 274, 4504-4512; Yeh, J. C. et al, 1999, J. Biol. Chem. 274, 3215-3221; and Schwientek et al, 2000, J. Biol. Chem. 275, 11106-11113). Acceptors for Core 2 GnT-M include oligosaccharides, glycoproteins, O-linked core 1-glycopeptides, and glycosphingolipids comprising the sequences Galβ1-3GalNAc, or Glcβ1-3GalNAc. Acceptors for Core 2 GnT3 include oligosaccharides, glycoproteins, O-linked core 1 and core 3-glycopeptides, and glycosphingolipids comprising the sequences Galβ1-3GalNAc, GlcNAc 1-3GalNAc, or Glc 1-3GalNAc.

In preferred embodiments of the invention, the glycosyltransferases are GnT1, GnTV, Core 2L/T1, Core 2b/T2/M, Core 2c/T3, and Core 3; and the invention provides preferred models and structures for these enzymes a methods of using the models and structures.

A glycosyltransferase or part thereof in the present invention may be a wild type enzyme, or part thereof, or a mutant, variant or homologue of such an enzyme.

The term “wild type” refers to a polypeptide having a primary amino acid sequence which is identical with the native enzyme (for example, the mammalian enzyme).

The term “mutant” refers to a polypeptide having a primary amino acid sequence which differs from the wild type sequence by one or more amino acid additions, substitutions or deletions. Preferably, the mutant has at least 90% sequence identity with the wild type sequence. Preferably, the mutant has 20 mutations or less over the whole wild-type sequence. More preferably the mutant has 10 mutations or less, most preferably 5 mutations or less over the whole wild-type sequence. A mutant may or may not be functional.

The term “variant” refers to a naturally occurring polypeptide which differs from a wild-type sequence. A variant may be found within the same species (i.e. if there is more than one isoform of the enzyme) or may be found within a different species. Preferably the variant has at least 90% sequence identity with the wild type sequence. Preferably, the variant has 20 mutations or less over the whole wild-type sequence. More preferably, the variant has 10 mutations or less, most preferably 5 mutations or less over the whole wild-type sequence.

The term “part” indicates that the polypeptide comprises a fraction of the wild-type amino acid sequence. It may comprise one or more large contiguous sections of sequence or a plurality of small sections. The “part” may comprise a ligand binding domain as described herein. The polypeptide may also comprise other elements of sequence, for example, it may be a fusion protein with another protein. Preferably the polypeptide comprises at least 50%, more preferably at least 65%, most preferably at least 80% of the wild-type sequence.

The term “homologue” means a polypeptide having a degree of homology with the wild-type amino acid sequence. The term “homology” can be equated with “identity”.

In the present context, a homologous sequence is taken to include an amino acid sequence which may be at least 75, 85 or 90% identical, preferably at least 95 or 98% identical to the wild-type sequence. Typically, the homologues will comprise the same sites (for example, ligand binding domains) as the subject amino acid sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences (e.g. Wilbur, W. J. and Lipman, D. J. Proc. Natl. Acad. Sci. USA (1983), 80:726-730).

The term “function” refers to the ability of a modulator to enhance or inhibit the association between a glycosyltransferase and a ligand or substrate, or the activity of the glycosyltransferase.

“Ligand binding domain” refers to a region of a molecule or molecular complex that as a result of its shape, favourably associates with a ligand or a part thereof. For example, it may be a region of a glycosyltransferase that is responsible for binding a ligand, substrate, or known modulator. With reference to the models and structures of the invention, residues in a ligand binding domain may be defined by their spatial proximity to a ligand in the model or structure.

The term “ligand binding domain” includes homologues of a ligand binding domain or portions thereof. As used herein, the term “homologue” in reference to a ligand binding domain refers to a ligand binding domain or a portion thereof which may have deletions, insertions or substitutions of amino acid residues as long as the binding specificity of the molecule is retained. In this regard, deliberate amino acid substitutions may be made on the basis of similarity in polarity, charge, solubility, hydrophobicity, hydrophilicity, and/or the amphipathic nature of the residues as long as the binding specificity of the ligand binding domain is retained.

As used herein, the term “portion thereof” means the structural coordinates corresponding to a sufficient number of amino acid residues of a glycosyltransferase ligand binding domain (or homologues thereof) that are capable of associating or interacting with a ligand, substrate, modulator, or test compound that binds to the ligand binding domain. This term includes glycosyltransferase ligand binding domain amino acid residues having amino acid residues from about 4 Å to about 5 Å of a bound compound or fragment thereof. Thus, for example, the structural coordinates provided in the structure may contain a subset of the amino acid residues in the ligand binding domain which may be useful in the modeling and design of compounds that bind to the ligand binding domain.

A ligand binding domain may be defined by its association with a ligand. With reference to the structures and models of the invention, residues in the ligand binding domain may be defined by their spatial proximity to a ligand. For example, such may be defined by their proximity to a substrate or modulator.

“Ligand” refers to a compound or entity that associates with a ligand binding domain, including substrates or analogues or parts thereof. A ligand may be designed rationally using a model according to the invention. A ligand may be a modulator.

“Modulator” refers to a molecule which changes or alters the biological activity of a glycosyltransferase. A modulator may increase or decrease glycosyltransferase activity, or change its characteristics, or functional or immunological properties. It may be an inhibitor that decreases the biological or immunological activity of the protein. A modulator may include but is not limited to peptides, members of random peptide libraries and combinatorial chemistry-derived molecular libraries, phosphopeptides (including members of random or partially degenerate, directed phosphopeptide libraries), antibodies, carbohydrates, monosaccharides, oligosaccharides, polysaccharides, glycolipids, heterocyclic compounds, nucleosides or nucleotides or parts thereof, and small organic or inorganic molecules. A modulator may be an endogenous physiological compound or it may be a natural or synthetic compound. The term “modulator” also includes a chemically modified ligand or compound, and includes isomers and racemic forms.

The term “structural coordinates” as used herein refers to a set of values that define the position of one or more amino acid residues or molecules with reference to a system of axes. A data set of structural coordinates defines the three dimensional structure of a molecule or molecules. Structural coordinates can be slightly modified and still render nearly identical three dimensional structures. A measure of a unique set of structural coordinates is the root-mean-square deviation of the resulting structure. Structural coordinates that render three dimensional structures that deviate from one another by a root-mean-square deviation of less than 2 Å, preferably less than 0.5 Å, more preferably less than 0.3 Å, may be viewed by a person of ordinary skill in the art as identical.

Variations in structural coordinates may be generated because of mathematical manipulations of the structural coordinates of a glycosyltransferase described herein. For example, the structural coordinates of Tables 1-8 and 14-23 may be manipulated by crystallographic permutations of the structural coordinates, fractionalization of the structural coordinates, integer additions or subtractions to sets of the structural coordinates, inversion of the structural coordinates or any combination of the above.

Variations in structure due to mutations, additions, substitutions, and/or deletions of the amino acids, or other changes in any of the components that make up a structure of the invention may also account for modifications in structural coordinates. If such modifications are within an acceptable standard error as compared to the original structural coordinates, the resulting structure may be the same. Therefore, a ligand that bound to a ligand binding domain of a glycosyltransferase would also be expected to bind to another ligand binding domain whose structural coordinates defined a shape that fell within the acceptable error. Such modified structures of a ligand binding domain are also within the scope of the invention.

Various computational analyses may be used to determine whether a ligand or a ligand binding domain thereof is sufficiently similar to all or parts of a ligand or ligand binding domain of the invention. Such analyses may be carried out using conventional software applications and methods as described herein.

The term “modeling” includes the quantitative and qualitative analysis of molecular structure and/or function based on atomic structural information and interaction models. The term includes conventional numeric-based molecular dynamic and energy minimization models, interactive computer graphic models, modified molecular mechanics models, distance geometry, and other structure-based constraint models. Preferably modeling is performed using a computer and may be optimized using known methods. This is called modeling optimization.

The term “substrate” refers to molecules that associate with a glycosyltransferase as it catalyzes the transfer of a selected sugar from a nucleotide sugar donor to an acceptor that leads to the formation of a new glycosidic linkage. A substrate includes the nucleotide sugar donor and acceptor and parts thereof.

A “sugar nucleotide donor” refers to a nucleotide coupled to a selected sugar that is transferred by a glycosyltransferase to an acceptor. The selected sugar may be a monosaccharide or disaccharide, preferably a monosaccharide. A suitable selected sugar includes GlcNAc. The GlcNAc may be modified for example, the hydroxyls may be blocked with acetonide, acylated, or alkylated or substituted with other groups such as halogen. The nucleotide is preferably UDP. The heterocyclic amine base in the nucleotide may be modified. For example, when the base is uridine it may be modified at the C-5 or C-6 position with groups including but not limited to alkyl, aryl, gallic acid, and with electron donating and electron withdrawing groups. The sugar in the nucleotide (e.g. ribose) may be modified at the 2′ or 3′ position with groups including but not limited to alkyl, aryl, gallic acid, and with electron donating and electron withdrawing groups.

An “acceptor” refers to the part of a carbohydrate structure (e.g. glycoprotein, glycolipid) where the selected sugar of a sugar nucleotide donor is transferred by the glycosyltransferase.

The term “alkyl”, alone or in combination, refers to a branched or linear hydrocarbon radical, typically containing from 1 through 20 carbon atoms, preferably 1 through 10 carbon atoms, more preferably 1 to 6 carbon atoms. Typical alkyl groups include but are not limited to methyl, ethyl, 1-propyl, 2-propyl, 1-butyl, 2-butyl, tert-butyl, pentyl, hexyl, heptyl, octyl, nonyl, decyl, and the like.

The term “alkenyl”, alone or in combination, refers to an unsaturated branched or linear group typically having from 2 to 20 carbon atoms and at least one double bond. Examples of such groups include but are not limited to ethenyl, 1-propenyl, 2-propenyl, 1-butenyl, 1,3-butadienyl, 1-bexenyl, 2-hexenyl, 1-pentenyl, 2-pentenyl like.

The term “alkynyl”, alone or in combination, refers to an unsaturated branched or linear group having 2 to 20 carbon atoms and at least one triple bond. Examples of such groups include but are not limited to ethynyl, 1-propynyl, 2-propynyl, 1-butynyl, 2-butynyl, 1-pentenyl, and the like.

The term “cycloalkyl” refers to cyclic hydrocarbon groups and includes but is not limited to cyclopropyl, cyclobutyl, cyclopentyl, cyclohexyl, cycloheptyl, and cyclooctyl.

The term “aryl”, alone or in combination, refers to a monocyclic or polycyclic group, preferably a monocyclic or bicyclic group. An aryl group may optionally be substituted as described herein. Examples of aryl groups and substituted aryl groups are phenyl, benzyl, p-nitrobenzyl, p-methoxybenzyl, biphenyl, and naphthyl.

The term “alkoxy” alone or in combination, refers to an alkyl or cycloalkyl linked to the parent molecular moiety through an oxygen atom. The term “aryloxy” refers to an aryl linked to the parent molecular moiety through an oxygen atom. Examples of alkoxy groups are methoxy, ethoxy, propoxy, vinyloxy, allyloxy, butoxy, pentoxy, hexoxy, cyclopentoxy, and cyclohexoxy. Examples of aryloxy groups are phenyloxy, O-benzyl i.e. benzyloxy, O-p-nitrobenzyl and O-p-methyl-benzyl, 4-nitrophenyloxy, 4-chlorophenyloxy, and the like.

The term “halo” or “halogen”, alone or in combination, means fluoro, chloro, bromo, or iodo.

The term “amino”, alone or in combination, refers to a chemical functional group where a nitrogen atom (N) is bonded to three substituents being any combination of hydrogen, alkyl, cycloalkyl, alkenyl, alkynyl, or aryl with the general chemical formula —NR₁₄R,₁₆ where R₁₄ and R₁₆ can be any combination of hydrogen, alkyl, cycloalkyl, alkenyl, alkynyl, or aryl. Optionally one substituent on the nitrogen atom can be a hydroxyl group (—OH) to give an amine known as a hydroxylamine. Examples of amino groups are amino (—NH₂), methylamine, ethylamine, dimethylamine, 2-propylamine, butylamine, isobutylamine, cyclopropylamine, benzylamine, allylamine, hydroxylamine, cyclohexylamino (—NHCH(CH₂)₅), piperidine (—N(CH₂)₅) and benzylamino (—NHCH2C₆H₅).

The term “thioalkyl”, alone or in combination, refers to a chemical functional group where a sulfur atom (S) is bonded to an alkyl. Examples of thioalkyl groups are thiomethyl, thioethyl, and thiopropyl.

The term “thioaryl”, alone or in combination, refers to a chemical functional group where a sulfur atom (S) is bonded to an aryl group with the general chemical formula —SR₁₆ where R₁₆ is an aryl group which may be substituted. Examples of thioaryl groups and substituted thioaryl groups are thiophenyl, para-chlorothiophenyl, thiobenzyl, 4-methoxy-thiophenyl, 4-nitro-thiophenyl, and para-nitrothiobenzyl.

Heterocyclic rings are molecular rings where one or more carbon atoms have been replaced by hetero atoms (atoms not being carbon) such as for example, oxygen (O), nitrogen (N) or sulfur (S), or combinations thereof. Examples of heterocyclic rings include ethylene oxide, tetrahydrofuran, thiophene, piperidine (piperidinyl group), pyridine (pyridinyl group), and caprolactam. A carbocyclic or heterocyclic group may be optionally substituted at carbon or nitrogen atoms with for example, alkyl, phenyl, benzyl or thienyl, or a carbon atom in the heterocyclic group together with an oxygen atom may form a carbonyl group, or a heterocyclic group may be fused with a phenyl group.

“Antibody” includes intact monoclonal or polyclonal molecules, and immunologically active fragments (e.g. a Fab or (Fab)₂ fragment), an antibody heavy chain, an antibody light chain, a genetically engineered single chain Fᵥ molecule (Ladner et al, U.S. Pat. No. 4,946,778), a humanized antibody or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. Antibodies that bind a peptide of the invention can be prepared using intact peptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, and keyhole limpet hemocyanin. The coupled peptide may then be used to immunize the animal (e.g., a mouse, a rat, or a rabbit).

By being “derived from” a ligand binding domain is meant any molecular entity which is identical or substantially equivalent to a native ligand binding domain of a molecule i.e. a loop structure of a glycosyltransferase. A peptide derived from a specific ligand binding domain may encompass the amino acid sequence of a naturally occurring ligand binding domain, any portion of that domain, or other molecular entity that functions to associate with an associated molecule. A peptide derived from such a ligand binding domain will interact directly or indirectly with an associated molecule in such a way as to mimic a native ligand binding domain. Such peptides may include competitive inhibitors, peptide mimetics, and the like.

“Peptide mimetics” are structures which serve as substitutes for peptides in interactions between molecules (See Morgan et al (1989), Ann. Reports Med. Chem. 24:243-252 for a review ). Peptide mimetics include synthetic structures which may or may not contain amino acids and/or peptide bonds but retain the structural and functional features of a peptide, or agonist, or antagonist. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide, or agonist or antagonist of the invention.

### Three Dimensional Structure of Glycosyltransferases and Binding Domains of Same

The present invention provides a glycosyltransferase secondary, tertiary and/or quanternary structure. The invention also provides a homology model that represents the secondary, tertiary, and/or quantemary structure of a glycosyltransferase. A model may, for example, be a structural model (or representation thereof), or a computer model. The model itself may be in two or three dimensions. It is possible for a computer model to be in three dimensions despite the constraints imposed by a conventional computer screen, if it is possible to scroll along at least a pair of axes, causing “rotation” of the image. A model or structure of a glycosyltransferase may be defined by the structural coordinates of each of Tables 1 through 8.

In accordance with an aspect of the invention a method is provided for designing a homology model of a ligand binding domain of a glycosytransferase wherein the homology model may be displayed as a three-dimensional image, the method comprising:

(i) providing an amino acid sequence and structural coordinates of a ligand binding domain structure of a glycosyltransferase, preferably a GnT1 glycosytransferase;

(ii) modifying said structure to take into account differences between the amino acid configuration of the ligand binding domains of the glycosyltransferase on the one hand and the glycosyltransferase on the other hand to generate a homology model, and

(iii) if required refining the homology model.

The method may further comprise comparing the homology model with the structures of other, similar, proteins.

Models or structures of preferred glycosyltransferases of the invention comprise the following atomic structural coordinates:

Table 1—Structural coordinates for GnT1.

Tab]e 2—Structural coordinates for GnTV.

Table 3—Structural coordinates for core 2L or T1 (human).

Table 4—Structural coordinates for core 2L or T1(mouse)

Table 5—Structural coordinates for core 2L (bovine)

Table 6—Structural coordinates for core 2b/core M/core 2 T2.

Table 7—Structural coordinates for core 2C (human)

Table 8—Structural coordinates for core 3.

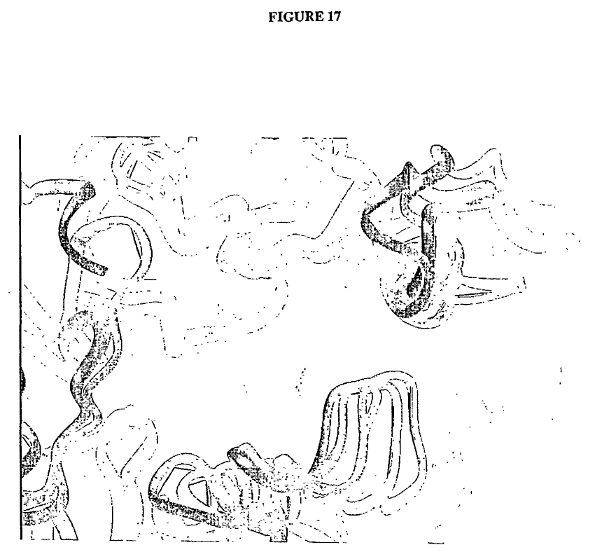

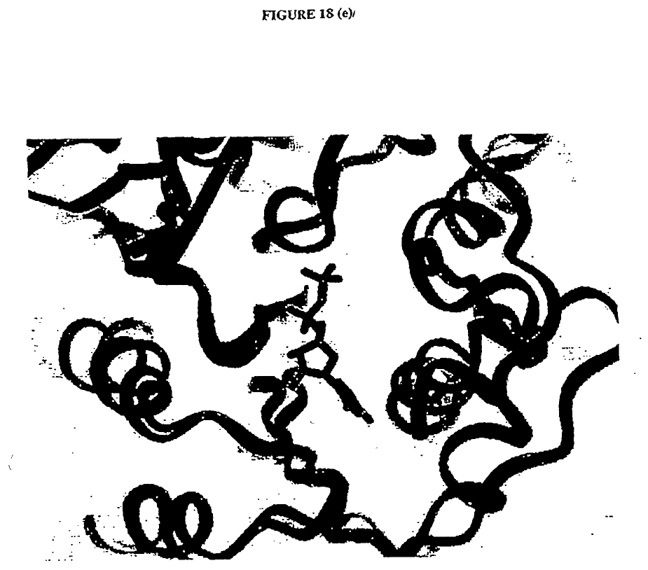

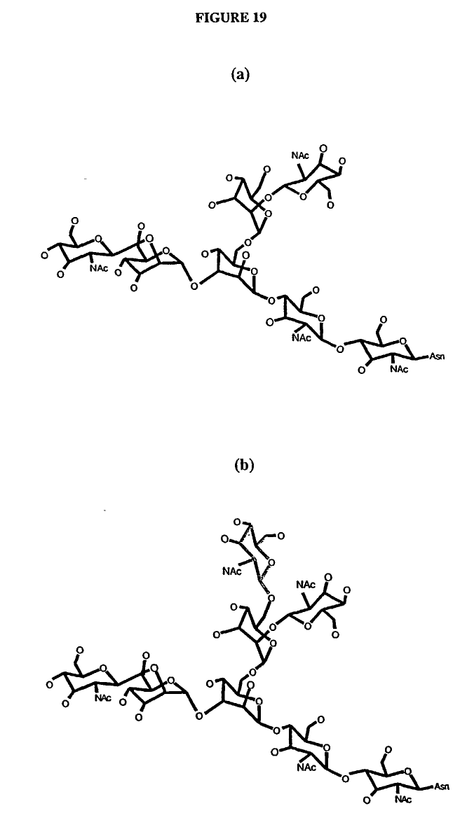

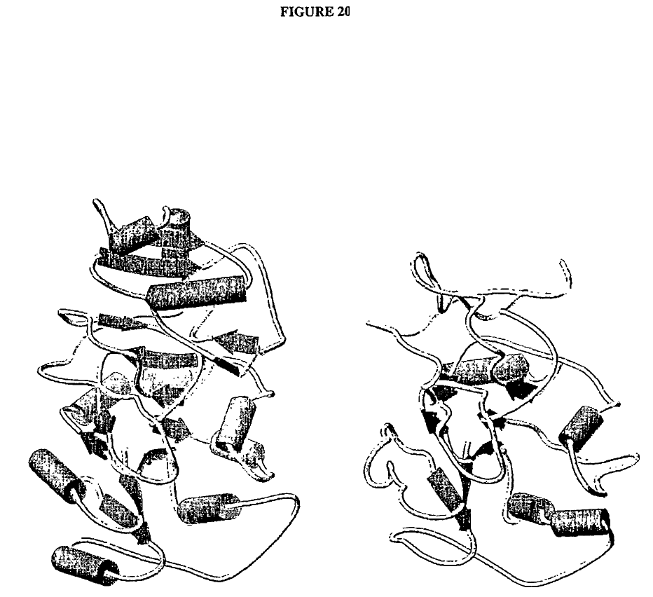

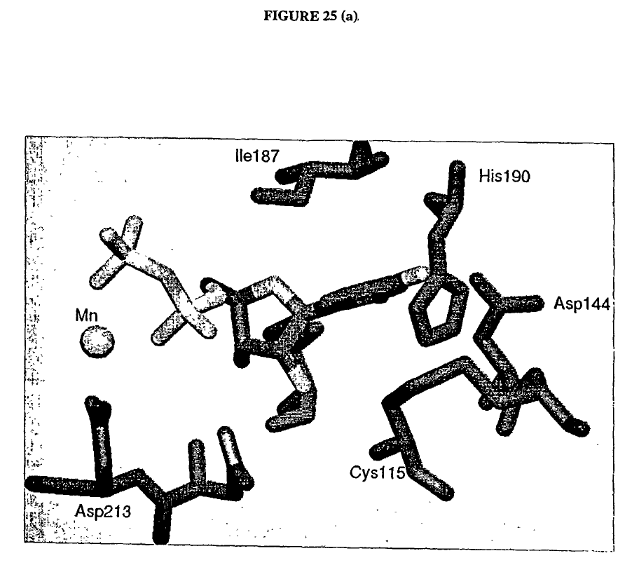

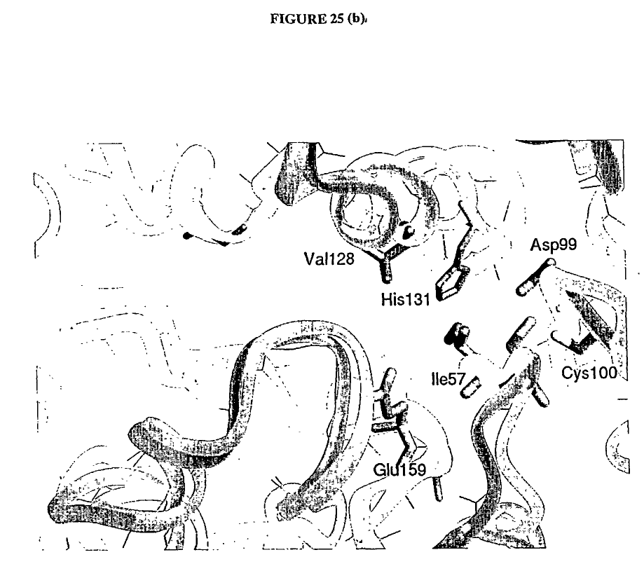

Computer representations of exemplary structures or models of the invention are illustrated in the FIGS. For example, FIG. 6 illustrates homology models for GnTV, Core 2L, and Core 2b/M; FIG. 17 shows a superimposition of main chain atoms of various structures; FIG. 20 shows a homology model of GnTV; and FIG. 25b shows a Core 2L model.

The structural coordinates in a structure or model of the invention may comprise the amino acid residues of a glycosyltransferase ligand binding domain, or a portion or homolog thereof useful in the modeling and design of test compounds capable of binding to the glycosyltransferase. Therefore, the invention also relates to a secondary, tertiary, or quantemary structure or model of a ligand binding domain of a glycosyltransferase. Ligand binding domains include the ligand binding domains for a disphosphate group of a sugar nucleotide donor, a nucleotide of a sugar nucleotide donor, a nitrogeneous heterocyclic base (preferably a pyrimidine base, more preferably uracil) of a sugar nucleotide donor, and/or a sugar (e.g. GlcNAc) of a sugar nucleotide donor.

A structure of a ligand binding domain may be defined by selected atomic interactions or contacts in the ligand binding domain, as follows:

(a) one or more of atomic interactions or atomic contacts for GnT1 shown in Table 10;

(b) one or more of atomic interactions or atomic contacts for GnTV shown in Table 11;

(c) one or more of atomic interactions or atomic contacts for Core 2L/T1 shown in Table 12; or

(d) one or more of atomic interactions or atomic contacts for Core 2b/T shown in Table 13.

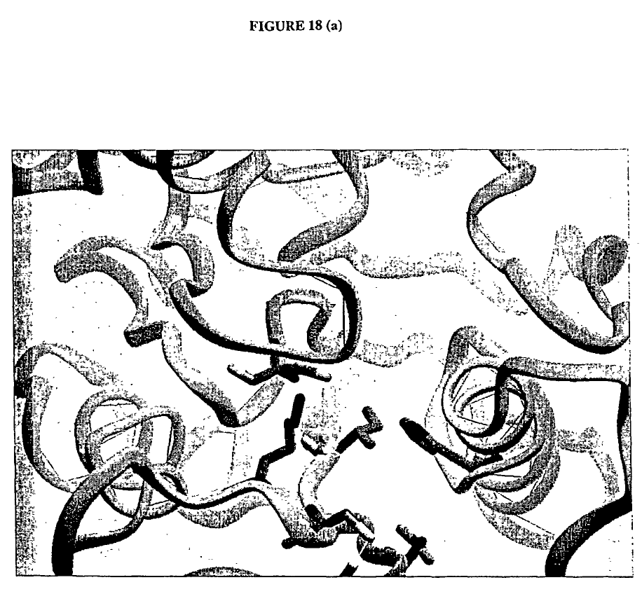

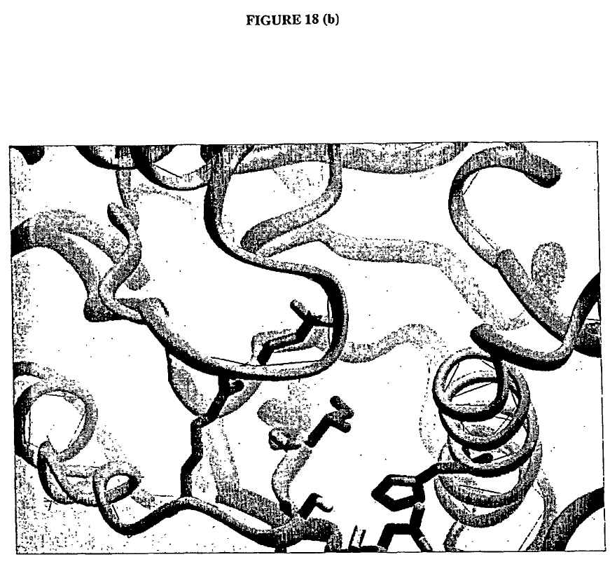

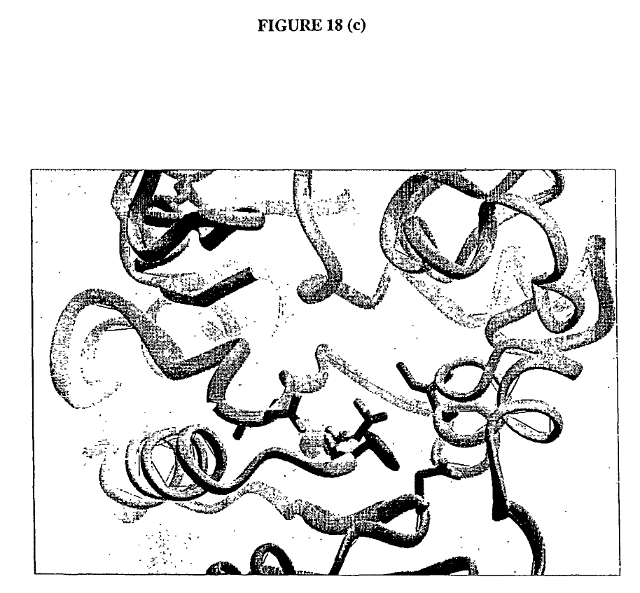

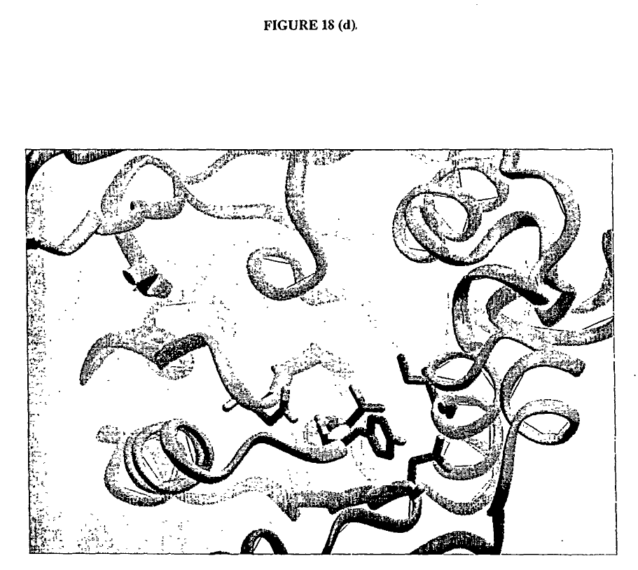

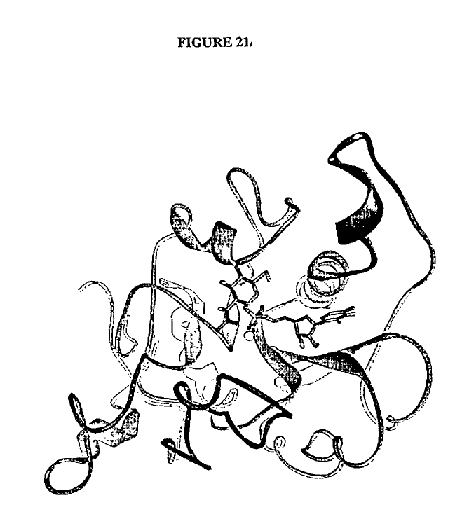

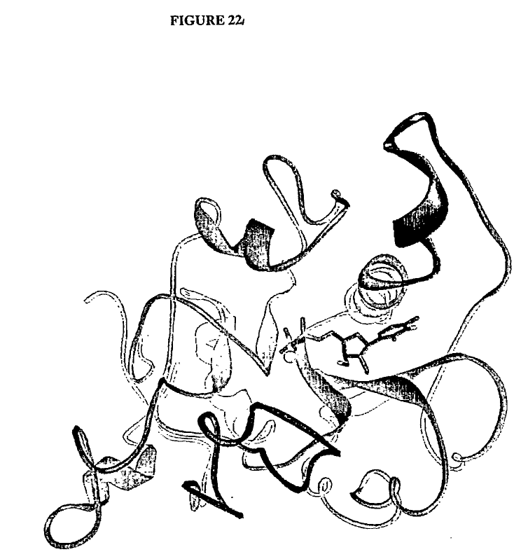

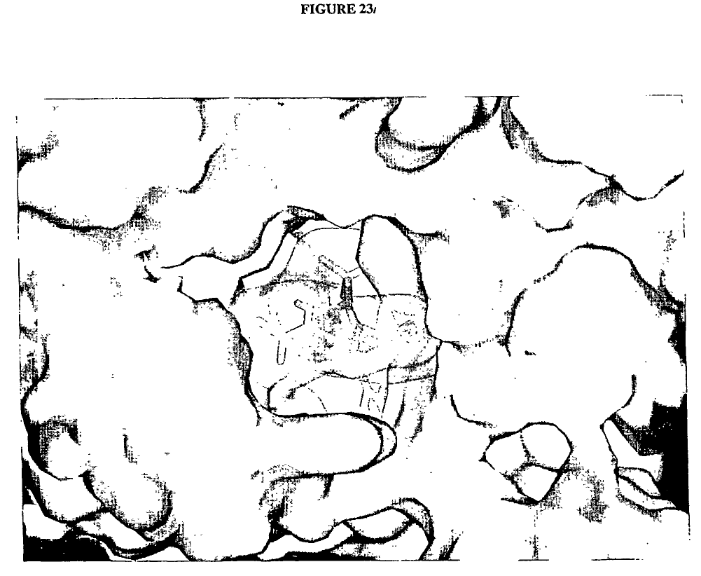

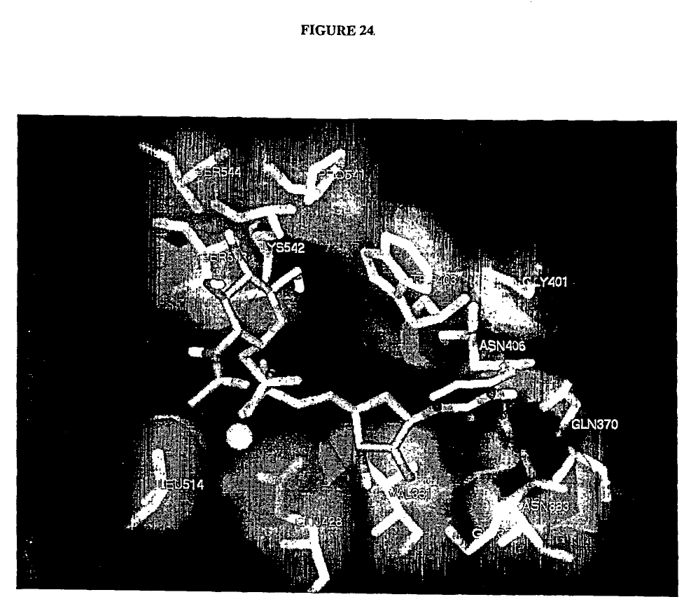

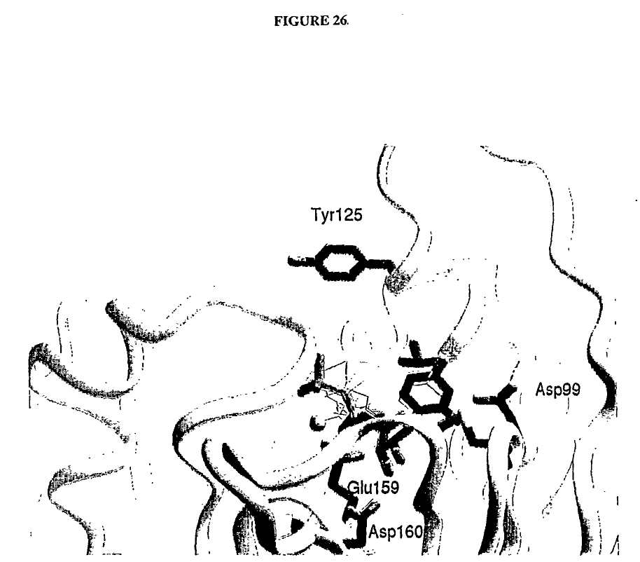

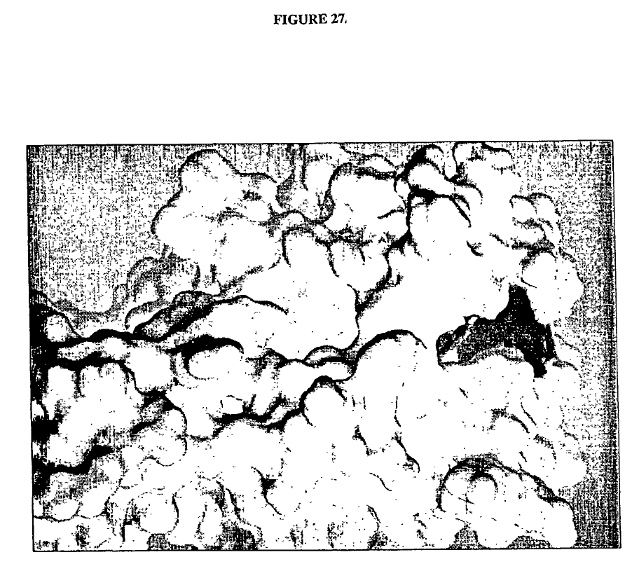

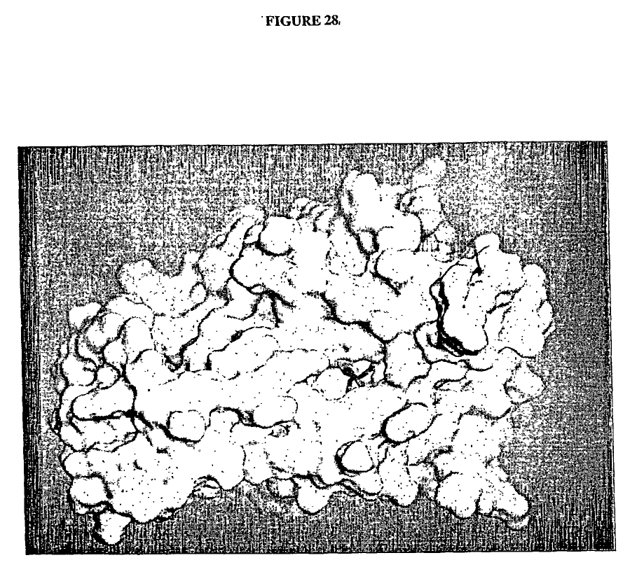

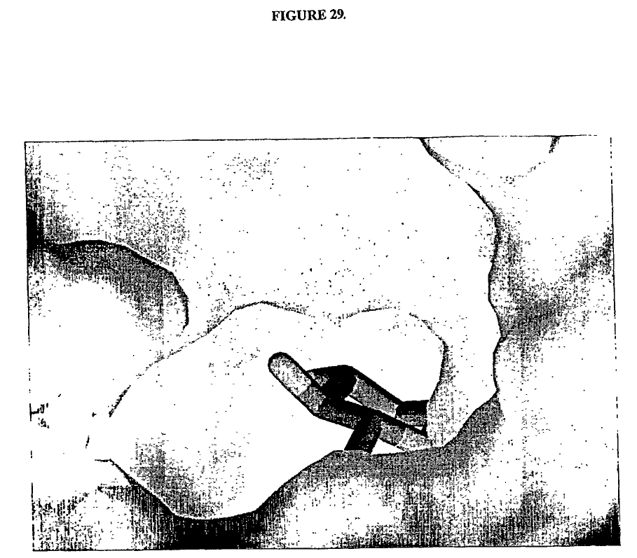

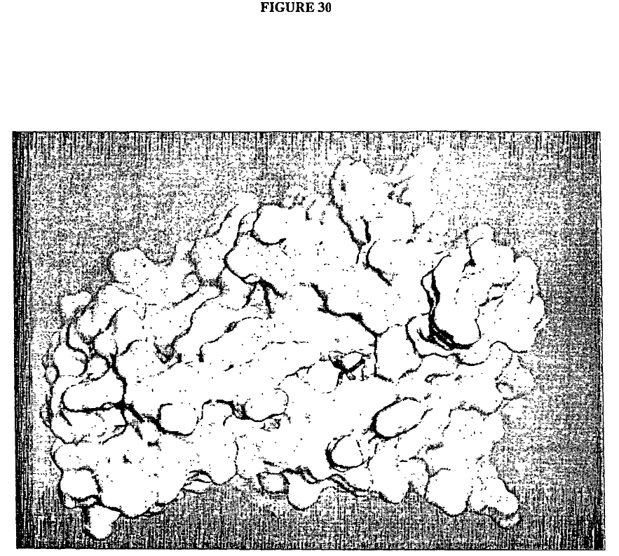

Computer representations of exemplary structures or models of ligand binding domains (and ligands) of the invention are illustrated in the Figures. For example, FIGS. 18, 22, 25a, 26, and 27 show models of a UDP ligand binding domain; FIGS. 21, 23, 24, 28 show models of a UDP-GlcNAc ligand binding domain; and FIG. 29 shows a sugar ligand binding domain.

It is understood that a structure or model of the invention includes a structure where at least one amino acid residue is replaced with a different amino acid residue or by adding or deleting amino acid residues, and having substantially the same three dimensional structure as the glycosyltransferase as described herein, or the ligand binding domains as described herein, i.e. having a set of atomic structural coordinates that have a root mean square deviation of less than or equal to about 2 Å, preferably less than 0.5 Å, most preferably less than 0.3 Å, when superimposed with the atomic structure coordinates of a glycosyltransferase as described herein or a ligand binding domain as described herein when at least 50% to 100% of the atoms of the ligand binding domain or binding domains of components thereof as the case may be, are included in the superimposition.

The invention also features a secondary, tertiary, or quantemary structure or model of a glycosyltransferase in association with one or more molecules (e.g. substrates such as UDP-GlcNac, uridine-ribose, monophophate-Mn²⁺, or diphosphate-Mn²⁺). The association may be covalent or non-covalent. The molecule may be any organic molecule, and it may modulate the function of a glycosyltransferase by, for example, inhibiting or enhancing its function, or it may be an acceptor or donor for the glycosyltransferase. It is preferred that the geometry of the compound and the interactions formed between the compound and the glycosyltransferase provide high affinity binding between the two molecules.

The structure and model of a glycosyltransferase described herein has allowed the identification and characterization of ligand binding domains of UDP and UDP-GlcNAc. The UDP-GlcNAc binding domain has been subdivided into sub-sites (the uracil binding domain, ribose binding domain, pyrophosphate binding domain, GlcNAc binding domain) and characterized.

In an embodiment of the invention, a secondary, tertiary, and/or quanternary structure or model of a ligand binding domain of a glycosyltransferase that associates with a diphosphate of a sugar nucleotide donor is provided comprising (a) atomic interaction 7 listed in Table 10 (GnTI Table); (b) at least two of atomic interactions 9, 10, 11, 12, and 13 listed in Table 12 (Core 2L Table); (c) at least two of atomic interactions 11, 12, 13, 14, or 15 listed in Table 13 (Core2b/M); or (d) atomic interaction 8 listed in Table 11 (GNTV Table), each atomic interaction defined therein by a residue (more preferably a specific atom where indicated) on the diphosphate of the sugar nucleotide donor and an amino acid, (more preferably a specific atom where indicated), on the glycosyltransferase. Preferably, a ligand binding domain is defined by atomic interaction 7 listed in Table 10 (GnT1 Table); atomic interactions 9, 10, 11, 12, and 13 listed in Table 12 (Core 2L Table), atomic interactions 11, 12, 13, 14, and 15 listed in Table (Core2b/M), or atomic interaction 8 listed in Table 11 (GNTV Table). Most preferably, a ligand binding domain is defined by the atoms of the amino acid residues of the atomic interactions having the structural coordinates for the atoms listed in Table 1 for GnT1, Table 3, 4, or 5 for Core 2L, Table 6 for Core 2b(M), and Table 2 for GnTV.

The three dimensional structure of a complex of a ligand binding domain of a glycosyltransferase in association with a disphosphate can also be defined as described above.

In an embodiment of the invention, a secondary, tertiary, and/or quanternary structure or model of a ligand binding domain of a glycosyltransferase that associates with a heterocyclic amine base (preferably uracil) of a sugar nucleotide donor is provided comprising at least two of the following atomic interactions (a) 1, 2, 3, and 4 listed in Table 10 (GnTI Table); (b) 1, 2, 3, 4, and 5 listed in Table 12 (Core 2L Table); (c) 1, 2, 3, and 4 listed in Table (13) (Core2b/M); or (d) 1, 2, 3, and 4 listed in Table 11 (GNTV Table), each atomic interaction defined therein by a residue (more preferably a specific atom where indicated) on the heterocyclic amine base of the sugar nucleotide donor and an amino acid, (more preferably a specific atom where indicated), on the glycosyltransferase. Preferably, a ligand binding domain is defined by atomic interactions 1, 2, 3, and 4 listed in Table 10 (GnT1 Table); atomic interactions 1, 2, 3, 4, and 5, listed in Table 12 (Core 2L Table), atomic interactions 1, 2, 3, and 4 listed in table 13 (Core2b/M), or atomic interactions 1, 2, 3, and 4, listed in Table 11 (GNTV Table). Most preferably, a ligand binding domain is defined by the atoms of the amino acid residues in the atomic interactions having the structural coordinates for the atoms listed in Table 1 for GnT1, Table 3, 4, or 5 for Core 2L, Table 6 for Core 2b(M), and Table 2 for GnTV. The three dimensional structure of a complex of a ligand binding domain of a glycosyltransferase in association with a heterocyclic amine base (preferably uracil) can also be defined as described above.

In an embodiment of the invention, a secondary, tertiary, and/or quantemary structure or model of a ligand binding domain of a glycosyltransferase that associates with the sugar (preferably ribose) of the nucleotide of a sugar nucleotide donor is provided comprising atomic interaction 5 or 6 listed in Table 10 (GnT1 Table); at least two of atomic interactions 6, 7, and 3 listed in Table 12 (Core 2L Table), or atomic interaction 5 listed in Table 11 (GnTV Table), each atomic interaction defined therein by a residue (more preferably a specific atom where indicated) on the sugar of the nucleotide of the sugar nucleotide donor and an amino acid, (more preferably a specific atom where indicated), on the glycosyltransferase. Preferably, a ligand binding domain is defined by atomic interactions 5 and 6 listed in Table 10 (GnTI Table); atomic interactions 6, 7, and 8 listed in Table 12 (Core 2L Table), or atomic interaction 5 listed in Table 11 (GNTV Table). Most preferably, a ligand binding domain is defined by the atoms of the amino acid residues in the atomic interactions having the structural coordinates for the atoms listed in Table 1 for GnT1, Table 3, 4, or 5 for Core 2L, Table 6 for Core 2b(M), and Table 2 for GnTV. The three dimensional structure of a complex of a ligand binding domain of a glycosyltransferase in association with a sugar (preferably ribose) can also be defined as described above.

In an embodiment of the invention, a secondary, tertiary, and/or quantemary structure or model of a ligand binding domain of a glycosyltransferase that associates with the sugar (GlcNAc) of a sugar nucleotide donor is provided comprising at least two of atomic interactions 8, 9, 10, 11, and 12 listed in Table 10 (GnT1 Table); at least two of atomic interactions 14, 15, 16, 17, and 18 listed in Table 12 (Core 2L Table), atomic interactions 16 or 17 listed in Table 13 (Core2b/M), or at least two of atomic interactions 9, 10, 11, 12, and 13 listed in Table 11 (GNTV Table), each atomic interaction defined therein by a residue (more preferably a specific atom where indicated) on the sugar of the sugar nucleotide donor and an amino acid, (more preferably a specific atom where indicated), on the glycosyltransferase. Preferably, the ligand binding domain is defined by atomic interactions 8, 9, 10, 11 and 12 listed in Table 10 (GnT1 Table); atomic interactions 14, 15, 16, 17, and 13 listed in Table 12 (Core 2L Table); atomic interactions 16 and 17 listed in Table 13 (Core2b/M), or atomic interactions 9, 10, 11, 12, and 13 listed in Table 11 (GNTV Table). Most preferably, a ligand binding domain is defined by the atoms of the amino acid residues in the atomic interactions having the structural coordinates for the atoms listed in Table 1 for GnT1, Table 3, 4, or 5 for Core 2L, Table 6 for Core 2b(M), and Table 2 for GnTV. The three dimensional structure of a complex of a ligand binding domain of a glycosyltransferase in association with a sugar (GlcNAc) of a sugar nucleotide donor can also be defined as described above.

A secondary, tertiary, and/or quanternary structure or model of a ligand binding domain of a glycosyltransferase that binds UDP is provided characterized by (a) a hydrogen bond between an Asp side chain of the glycosyltransferase with position 3 of the uracil ring of UDP; (b) a stacking interaction between either a disulfide or an aromatic group (Phe or Tyr) of the glycosyltransferase and the uracil ring of the UDP; (c) a stacking interaction between either an Ile or a Thr of the glycosyltransferase and the ribose ring of the UDP; and (d) metal mediated charge interactions between a well-conserved Asp/Glu of the glycosyltransferase and a pyrophosphate oxygen of the UDP.

In an embodiment of the invention, a secondary, tertiary, and/or quanternary structure or model of a ligand binding domain of a glycosyltransferase that associates with a nucleotide (preferably UDP) of a sugar nucleotide donor is provided comprising at least two of (a) atomic interactions 1, 2, 3, 4, 5, 6, and 7 listed in Table 10 (GnT1 Table); (b) atomic interactions 1, 2, 3, 4, 5, 6, 7, 8, 9, 10, 11, 12, and 13 listed in Table 12 (Core 2L Table); (c) atomic interactions 1, 2, 3, 4, 5, 6, 7, 8, 9, 10, 11, 12, 13, 14 and 15 listed in Table 13 (Core2b/M); or (d) atomic interactions 1, 2, 3, 4, 5, 6, 7, and 8, listed in Table 11 (GNTV Table), each atomic interaction defined therein by a residue (more preferably a specific atom where indicated) on the nucleotide of the sugar nucleotide donor and an amino acid, (more preferably a specific atom where indicated), on the glycosyltransferase. Preferably, a ligand binding domain is defined by atomic interactions 1, 2, 3, 4, 5, 6, and 7 listed in Table 10 (GnT1 Table); atomic interactions 1, 2, 3, 4, 5, 6, 7, 8, 9, 10, 11, 12, and 13 listed in Table 12 (Core 2L Table); atomic interactions 1 2, 3, 4, 5, 6, 7, 8, 9, 10, 11, 12, 13, 14, and 15 listed in Table 13 (Core2b/M); or, atomic interactions 1, 2, 3, 4, 5, 6, 7, and 8 listed in Table 11 (GNTV Table). Most preferably, a ligand binding domain is defined by the atoms of the amino acid residues in the atomic interactions having the structural coordinates for the atoms listed in Table 1 for GNT1, Table 3, 4, or 5 for Core 2L, Table 6 for Core 2b(M), and Table 2 for GnTV. The three dimensional structure of a complex of the ligand binding domain of a glycosyltransferase in association with a nucleotide (e.g. UDP) of a sugar nucleotide donor can also be defined as described above.

In an embodiment of the invention, a secondary, tertiary, and/or quantemary structure or model of a ligand binding domain of a glycosyltransferase that associates with a sugar nucleotide donor (e.g. UDP-GlcNAc) is provided comprising at least two of (a) atomic interactions 1, 2,3, 4, 5, 6, 7, 8, 9, 10, 11, and 12 listed in Table 10 (GnT1 Table); (b) atomic interactions 1, 2, 3, 4, 5, 6, 7, 8, 9, 10, 11, 12, 13, 14, 15, 16, 17, and 18 listed in Table 12 (Core 2L Table); (c) atomic interactions 1, 2, 3, 4, 5, 6, 9, 10, 11, 12, 13, 14, 15, 16, and 17 listed in Table 13 (Core2b/M), or (d) atomic interactions 1, 2, 3, 4, 5, 6, 7, 8, 9, 10, 11, 12, and 13 listed in Table 11 (GNTV Table), each atomic interaction defined therein by a residue (more preferably a specific atom where indicated) on the sugar nucleotide donor and an amino acid, (more preferably a specific atom where indicated), on the glycosyltransferase. Preferably, a ligand binding domain is defined by atomic interactions 1, 2, 3, 4, 5, 6, 7, 8, 9, 10, 11, and 12 listed in Table 10 (GnT1 Table); atomic interactions 1, 2, 3, 4, 5, 6, 7, 8, 9, 10, 11, 12, 13, 14, 15, 16, 17, and 18 listed in Table 12 (Core 2L Table); atomic interactions 1, 2, 3, 4, 5, 6, 9, 10, 11, 12, 13, 14, 15, 16, and 17 listed in Table 13 (Core2b/M); or atomic interactions 1, 2, 3, 4, 5, 6, 7, 8, 9, 10, 11, 12, and 13 listed in Table 11 (GNTV Table). Most preferably, a ligand binding domain is defined by the atoms of the amino acid residues in the atomic interactions having the structural coordinates for the atoms listed in Table 1 for GnT1, Table 3, 4, or 5 for Core 2L, Table 6 for Core 2b(M), and Table 2 for GnTV. The three dimensional structure of a complex of a ligand binding domain of a glycosyltransferase in association with a sugar nucleotide donor (e.g. UDP-GlcNAc) can also be defined as described above.

The three dimensional structure of glycostyltransferases are characterized by a “loop” structure. The loop folds on top of the pyrophosphate after the sugar nucleotide donor associates with the active site of the glycosyltransferase. The loop has a similar amino acid motif in glycosyltransferases but in Core 2 transferases the loop is hydrophilic and in GNTI and GNTV the loop is hydrophobic. Molecules that associate with the loop are highly specific inhibitors of the enzymes. In an embodiment of the invention, a secondary, tertiary, or quantemary structure or model of a loop structure of a glycosyltransferase that binds a pyrophosphate of a sugar nucleotide donor is provided comprising the structural coordinates for the loop structure of GnT1 listed in Table 21; Core 2L listed in Table 22; or GnTV listed in Table 23.

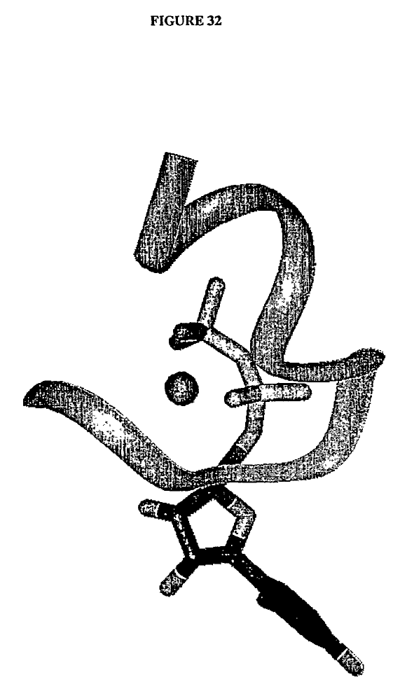

FIG. 32 illustrates a model of the pyrophosphate of UDP-GlcNAc interacting with the loop structure of Core 2L.

### Transition State Ligand Binding Domains

The invention also provides a secondary, tertiary, and/or quantemary structure or model of a ligand binding domain of a transition state of a reaction catalyzed by a glycosyltransferase. In particular, the invention provides a secondary, tertiary, and/or quanternary structure or model of a sugar transition state ligand binding domain, preferably a GlcNAc transition state ligand binding domain, of a glycosyltransferase comprising a hydrophobic pocket that is 1.9 to 3.5 Å, preferably 2.2 to 3.0 Å, from the pyrophosphate binding cavity for the glycosyltransferase The amino acid residues in the domain that associate with the C2 and C4 positions of the sugar preferably have the structural coordinates of Leu-331, and Leu 269 in Table 1 (GNT1 Table), or the structural coordinates of Leu -116 and Val-81 of Table 3, 4, or 5 (Core 2L coordinates).

The sugar transition ligand binding domain preferably comprises atomic interactions 14 to 18 in Table 12 (Core 2L Table) or atomic interactions 9 to 12 of Table 10 (GnT1 Table), or the particular structural coordinates for the atomic contacts of the atomic interactions as set out in Tables 1 (GnT1) or 3, 4, or 5 (Core 2L).

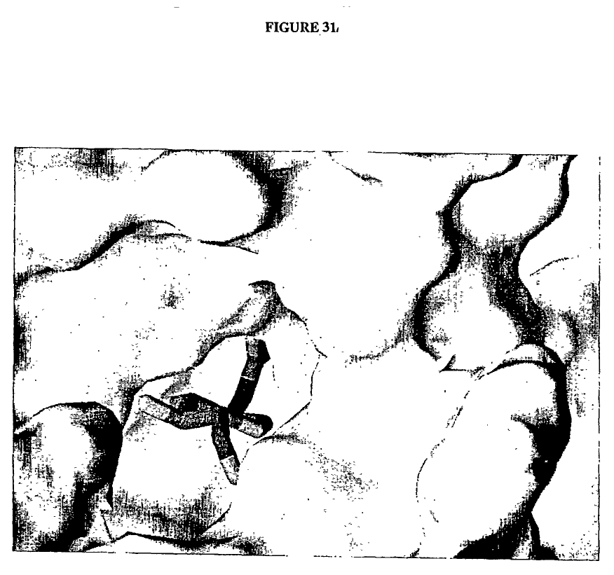

FIG. 31 shows a model of the binding of GlcNAc to the transition state of Core 2L showing a hydrophobic ligand binding domain.

### Identification of Homologues

The knowledge of the structures and models of the invention enables one skilled in the art to identify homologues of glycosyltransferases. This is achieved by searches of three-dimensional databases. Since structural folds are conserved to a greater extent than sequence, one may identify homologues with very little sequence identity or similarity. Programs that provide this type of database searching are known in the art and include Dal and the Fold recognition server located at UCLA (8). The structural coordinates of a protein structure are submitted and the program performs a multiple structural alignment with proteins in the protein data bank. Homologues identified in accordance with the present invention may be used in the methods of the invention described herein.

### Computer Format of Structures/Models

Information derivable from the structures of the present invention (for example the structural coordinates) or a model of the present invention may be provided in a computer-readable format.

Therefore, the invention provides a computer readable medium or a machine readable storage medium which comprises the models of the invention or structural coordinates of a glycosyltransferase including all or any parts of the glycosyltransferase (e.g ligand binding domain), ligands including portions thereof, or substrates including portions thereof. Such storage medium or storage medium encoded with these data are capable of displaying on a computer screen or similar viewing device, a three-dimensional graphical representation of a molecule or molecular complex which comprises the enzyme or ligand binding domains or similarly shaped homologous enzymes or ligand binding domains. Thus, the invention also provides computerized representations of a model or structure of the invention, including any electronic, magnetic, or electromagnetic storage forms of the data needed to define the structures such that the data will be computer readable for purposes of display and/or manipulation.

In an aspect the invention provides a computer for producing a model or three-dimensional representation of a molecule or molecular complex, wherein said molecule or molecular complex comprises a glycosyltransferase or ligand binding domain thereof defined by structural coordinates of glycosyltransferase amino acids or a ligand binding domain thereof, or comprises structural coordinates of atoms of a ligand or substrate, or a three-dimensional representation of a homologue of said molecule or molecular complex, wherein said computer comprises:

(a) a machine-readable data storage medium comprising a data storage material encoded with machine readable data wherein said data comprises the structural coordinates of glycosyltransferase amino acids according to any one of Tables 1-8 or a ligand binding domain thereof according to Table 21, 22, or 23, or a ligand according to any one of Tables 14-20;

(b) a working memory for storing instructions for processing said machine-readable data;

(c) a central-processing unit coupled to said working memory and to said machine-readable data storage medium for processing said machine readable data into said three-dimensional representation; and

(d) a display coupled to said central-processing unit for displaying said three-dimensional representation.

A homologue may comprise a glycosyltransferase or ligand binding domain thereof, or ligand or substrate that has a root mean square deviation from the backbone atoms of not more than 1.5 angstroms.

The invention also provides a computer for determining at least a portion of the structural coordinates corresponding to an X-ray diffraction pattern of a molecule or molecular complex wherein said computer comprises:

(a) a machine-readable data storage medium comprising a data storage material encoded with machine readable data wherein said data comprises the structural coordinates according to any one of Tables 1-8, and 14-23;

(b) a machine-readable data storage medium comprising a data storage material encoded with machine readable data wherein said data comprises an X-ray diffraction pattern of said molecule or molecular complex;

(c) a working memory for storing instructions for processing said machine-readable data of (a) and (b);

(d) a central-processing unit coupled to said working memory and to said machine-readable data storage medium of (a) and (b) for performing a Fourier transform of the machine readable data of (a) and for processing said machine readable data of (b) into structural coordinates; and

(e) a display coupled to said central-processing unit for displaying said structural coordinates of said molecule or molecular complex.

The invention also contemplates a computer programmed with a homology model of a ligand binding domain according to the invention; a machine-readable data-storage medium on which has been stored in machine-readable form a homology model of a ligand binding domain of a glycosyltransferase; and the use of a homology model as input to a computer programmed for drug design and/or database searching and/or molecular graphic imaging in order to identify new ligands or modulators for glycosyltransferases.

### Structural Determinations

The present invention also provides a method for determining the secondary and/or tertiary structures of a polypeptide by using a model according to the invention. The polypeptide may be any polypeptide for which the secondary and or tertiary structure is uncharacterised or incompletely characterised. In a preferred embodiment the polypeptide shares (or is predicted to share) some structural or functional homology to a glycosyltransferase. For example, the polypeptide may show a degree of structural homology over some or all parts of the primary amino acid sequence. For example the polypeptide may have one or more domains which show homology with a glycosyltransferase domain.

The polypeptide may be a glycosyltransferase with a different specificity for a ligand or substrate. The polypeptide may be a glycosyltransferase which requires a different metal cofactor. Alternatively (or in addition) the polypeptide may be a glycosyltransferase from a different species.

The polypeptide may be a mutant of the wild-type glycosyltransferase. A mutant may arise naturally, or may be made artificially (for example using molecular biology techniques). The mutant may also not be “made” at all in the conventional sense, but merely tested theoretically using the model of the present invention. A mutant may or may not be functional.

Thus, using a model of the present invention, the effect of a particular mutation on the overall two and/or three dimensional structure of a glycosyltransferase and/or the interaction between the enzyme and a ligand or substrate can be investigated. Alternatively, the polypeptide may perform an analogous function or be suspected to show a similar catalytic mechanism to the glycosyltransferase enzyme. For example, the polypeptide may transfer a sugar residue from a sugar nucleotide donor.

The polypeptide may also be the same as a polypeptide described herein, but in association with a different ligand (for example, modulator or inhibitor) or cofactor. In this way it is possible to investigate the effect of altering a ligand or compound with which the polypeptide is associated on the structure of a ligand binding domain.

Secondary or tertiary structure may be determined by applying the structural coordinates of the model of the present invention to other data such as an amino acid sequence, X-ray crystallographic diffraction data, or nuclear magnetic resonance (NMR) data. Homology modeling, molecular replacement, and nuclear magnetic resonance methods using these other data sets are described below.

Homology modeling (also known as comparative modeling or knowledge-based modeling) methods develop a three dimensional model from a polypeptide sequence based on the structures of known proteins (e.g. native or mutated glycosyltransferases). In the present invention the method utilizes a computer representation of the structure of a glycosyltransferase, or a binding domain or complex of same as described herein, a computer representation of the amino acid sequence of a polypeptide with an unknown structure (additional native or mutated glycosyltransferases), and standard computer representations of the structures of amino acids. The method in particular comprises the steps of; (a) identifying structurally conserved and variable regions in the known structure; (b) aligning the amino acid sequences of the known structure and unknown structure (c) generating coordinates of main chain atoms and side chain atoms in structurally conserved and variable regions of the unknown structure based on the coordinates of the known structure thereby obtaining a homology model; and (d) refining the homology model to obtain a three dimensional structure for the unknown structure. This method is well known to those skilled in the art (Greer, 1985, Science 228, 1055; Bundell et al 1988, Eur. J. Biochem. 172, 513; Knighton et al., 1992, Science 258:130-135, http:l/biochem.vt.edu/courses/modeling/homology.htn). Computer programs that can be used in homology modeling are Quanta and the Homology module in the Insight II modeling package distributed by Molecular Simulations Inc, or MODELLER (Rockefeller University, www.iucr.ac.uk/sinris-top/logical/prg-modeller.html).

In step (a) of the homology modeling method, a known glycosyltransferase structure is examined to identify the structurally conserved regions (SCRs) from which an average structure, or framework, can be constructed for these regions of the protein. Variable regions (VRs), in which known structures may differ in conformation, also must be identified. SCRs generally correspond to the elements of secondary structure, such as alpha-helices and beta-sheets, and to ligand- and substrate-binding sites (e.g. acceptor and donor binding sites). The VRs usually lie on the surface of the proteins and form the loops where the main chain turns.

Many methods are available for sequence alignment of known structures and unknown structures. Sequence alignments generally are based on the dynamic programming algorithm of Needleman and Wunsch [J. Mol. Biol. 48: 442-453, 1970]. Current methods include FASTA, Smith-Waterman, and BLASTP, with the BLASTP method differing from the other two in not allowing gaps. Scoring of alignments typically involves construction of a 20×20 matrix in which identical amino acids and those of similar character (i.e., conservative substitutions) may be scored higher than those of different character. Substitution schemes which may be used to score alignments include the scoring matrices PAM (Dayhoffet al., Meth. Enzymol. 91: 524-545, 1983), and BLOSUM (Henikoff and Henikoff, Proc. Nat. Acad. Sci. USA 89: 10915-0919, 1992), and the matrices based on alignments derived from three-dimensional structures including that of Johnson and Overington (JO matrices) (J. Mol. Biol. 233: 716-738, 1993).

Alignment based solely on sequence may be used, though other structural features also may be taken into account. In Quanta, multiple sequence alignment algorithms are available that may be used when aligning a sequence of the unknown with the known structures. Four scoring systems (i.e. sequence homology, secondary structure homology, residue accessibility homology, CA-CA distance homology) are available, each of which may be evaluated during an alignment so that relative statistical weights may be assigned.

When generating coordinates for the unknown structure, main chain atoms and side chain atoms, both in SCRs and VRs need to be modeled. A variety of approaches may be used to assign coordinates to the unknown. In particular, the coordinates of the main chain atoms of SCRs will be transferred to the unknown structure. VRs correspond most often to the loops on the surface of the polypeptide and if a loop in the known structure is a good model for the unknown, then the main chain coordinates of the known structure may be copied. Side chain coordinates of SCRs and VRs are copied if the residue type in the unknown is identical to or very similar to that in the known structure. For other side chain coordinates, a side chain rotamer library may be used to define the side chain coordinates. When a good model for a loop cannot be found fragment databases may be searched for loops in other proteins that may provide a suitable model for the unknown. If desired, the loop may then be subjected to conformational searching to identify low energy conformers if desired.

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R. et al, Nature 356: 83-85, 1992; and Bowie, J. U. et al, Science 253: 164-170, 1991]. Once any irregularities have been resolved, the entire structure may be further refined. Refinement may consist of energy minimization with restraints, especially for the SCRs. Restraints may be gradually removed for subsequent minimizations. Molecular dynamics may also be applied in conjunction with energy minimization.

The structural coordinates of a glycosyltransferase structure may be applied to nuclear magnetic resonance (NMR) data to determine the three dimensional structures of polypeptides in solution (e.g. additional native or mutated glycosyltransferases). (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide can guide the NMR spectroscopist to an understanding of the spatical interactions between secondary structural elements in a polypeptide of related structure. Information on spatial interactions between secondary structural elements can greatly simplify Nuclear Overhauser Effect (NOE) data from two-dimensional NMR experiments. In addition, applying the structural coordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of polypeptides with unknown structures, preferably a native or mutated glycosyltransferase, by applying the structural coordinates of a glycosyltransferase structure, or ligand binding domain or complex thereof described herein to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assignment” defines a method of analyzing NMR data and identifying which amino acids give rise to signals in the NMR spectrum.

### Screening Method

The present invention also provides a method of screening for a ligand that associates with a ligand binding domain and/or modulates the function of a glycosyltransferase, by using a structure or a model according to the present invention. The method may involve investigating whether a test compound is capable of associating with or binding a ligand binding domain.

In accordance with an aspect of the present invention, a method is provided for screening for a ligand capable of binding to a ligand binding domain, wherein said method comprises the use of a structure or model according to the invention.

In another aspect, the invention relates to a method of screening for a ligand capable of binding to a ligand binding domain, wherein the ligand binding domain is defined by the amino acid residue structural coordinates given herein, the method comprising contacting the ligand binding domain with a test compound and determining if said test compound binds to said ligand binding domain.

In one embodiment, the present invention provides a method of screening for a test compound capable of interacting with one or more key amino acid residue of the ligand binding domain of a glycosyltransferase.

Another aspect of the invention provides a process comprising the steps of:

(a) performing a method of screening for a ligand as described above;

(b) identifying one or more ligands capable of binding to a ligand binding domain; and

(c) preparing a quantity of said one or more ligands.

A further aspect of the invention provides a process comprising the steps of:

(a) performing the method of screening for a ligand as described above;

(b) identifying one or more ligands capable of binding to a ligand binding domain; and

(c) preparing a pharmaceutical composition comprising said one or more ligands.

Once a test compound capable of interacting with a key amino acid residue in a glycosyltransferase ligand binding domain has been identified, further steps may be carried out either to select and/or to modify compounds and/or to modify existing compounds, to modulate the interaction with key amino acid residues in the glycosyltransferase ligand binding domain.

Yet another aspect of the invention provides a process comprising the steps of:

(a) performing a method of screening for a ligand as described above;

(b) identifying one or more ligands capable of binding to a ligand binding domain;

(c) modifying said one or more ligands capable of binding to a ligand binding domain;

(d) performing said method of screening for a ligand as described above;

(e) optionally preparing a pharmaceutical composition comprising said one or more ligands.

As used herein, the term “test compound” means any compound which is potentially capable of associating with a ligand binding domain. If, after testing, it is determined that the test compound does bind to the ligand binding domain, it is known as a “ligand”.

A “test compound” includes, but is not limited to, a compound which may be obtainable from or produced by any suitable source, whether natural or not. The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal (particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptide synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The test compound may be screened as part of a library or a data base of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK (University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Test compounds may be tested for their capacity to fit spatially into a glycosyltransferase ligand binding domain. As used herein, the term “fits spatially” means that the three-dimensional structure of the test compound is accommodated geometrically in a glycosyltransferase ligand binding domain. The test compound can then be considered to be a ligand.

A favourable geometric fit occurs when the surface area of the test compound is in close proximity with the surface area of the cavity or pocket without forming unfavorable interactions. A favourable complementary interaction occurs where the test compound interacts by hydrophobic, aromatic, ionic, dipolar, or hydrogen donating and accepting forces. Unfavourable interactions may be steric hindrance between atoms in the test compound and atoms in the binding site.

In an embodiment of the invention, a method is provided for identifying potential modulators of a glycosyltransferase function. The method utilizes the structural coordinates or model of a glycosyltransferase three dimensional structure, or binding domain thereof. The method comprises the steps of (a) docking a computer representation of a test compound from a computer data base with a computer model of a ligand binding domain of a glycosyltransferase; (b) determining a conformation of a complex between the test compound and binding domain with a favourable geometric fit or favorable complementary interactions; and (c) identifying test compounds that best fit the glycosyltransferase ligand binding domain as potential modulators of glycosyltransferase function. The initial glycosyltransferase structure may or may not have substrates bound to it. A favourable complementary interaction occurs where a compound in a compound-glycosyltransferase complex interacts by hydrophobic, ionic, or hydrogen donating and accepting forces, with the active-site or binding domain of a glycosyltransferase without forming unfavorable interactions.

If a model of the present invention is a computer model, the test compounds may be positioned in a ligand binding domain through computational docking. If, on the other hand, the model of the present invention is a structural model, the test compounds may be positioned in the ligand binding domain by, for example, manual docking.

As used herein the term “docking” refers to a process of placing a compound in close proximity with a glycosyltransferase ligand binding domain, or a process of finding low energy conformations of a test compound/glycosyltransferase complex.

A screening method of the present invention may comprise the following steps:

(i) generating a computer model of a glycosyltransferase or a ligand binding domain thereof according to the first aspect of the invention;

(ii) docking a computer representation of a test compound with the computer model;

(iii) analysing the fit of the compound in the glycosyltransferase or ligand binding domain thereof.

In an aspect of the invention a method is provided comprising the following steps:

(a) docking a computer representation of a structure of a test compound into a computer representation of a ligand binding domain of a glycosyltransferase defined in accordance with the invention using a computer program, or by interactively moving the representation of the test compound into the representation of the binding domain;

(b) characterizing the geometry and the complementary interactions formed between the atoms of the ligand binding domain and the compound; optionally

(c) searching libraries for molecular fragments which can fit into the empty space between the compound and ligand binding domain and can be linked to the compound; and

(d) linking the fragments found in (c) to the compound and evaluating the new modified compound.

In an embodiment of the invention a method is provided which comprises the following steps:

(a) docking a computer representation of a test compound from a computer data base with a computer representation of a selected site (e.g. an inhibitor binding domain) on a glycosyltransferase structure or model defined in accordance with the invention to obtain a complex;

(b) determining a conformation of the complex with a favourable geometric fit and favourable complementary interactions; and

(c) identifying test compounds that best fit the selected site as potential modulators of the glycosyltransferase.

A method of the invention may be applied to a plurality of test compounds, to identify those that best fit the selected site.

The model used in the screening method may comprise a glycosyltransferase or ligand binding domain thereof either alone or in association with one or more ligands and/or cofactors. For example, the model may comprise a ligand binding domain in association with a ligand, substrate, or analogue thereof

If the model comprises an unassociated ligand binding domain, then the selected site under investigation may be the ligand binding domain itself. The test compound may, for example, mimic a known substrate for the enzyme in order to interact with the ligand binding domain. The selected site may alternatively be another site on the enzyme.

If the model comprises an associated ligand binding domain, for example a ligand binding domain in association with a ligand, substrate molecule or analogue thereof, the selected site may be the ligand binding domain or a site made up of the ligand binding domain and the complexed ligand, or a site on the ligand itself. The test compound may be investigated for its capacity to modulate the interaction with the associated molecule.

A test compound (or plurality of test compounds) may be selected on the basis of its similarity to a known ligand for the glycosyltransferase. For example, the screening method may comprise the following steps:

(i) generating a computer model of a glycosyltransferase ligand binding domain in complex with a ligand;

(ii) searching for a test compound with a similar three dimensional structure and/or similar chemical groups; and

(iii) evaluating the fit of the test compound in the ligand binding domain.

Searching may be carried out using a database of computer representations of potential compounds, using methods known in the art.

The present invention also provides a method for designing ligands for a glycosyltransferase. It is well known in the art to use a screening method as described above to identify a test compound with promising fit, but then to use this test compound as a starting point to design a ligand with improved fit to the model. A known modulator can also be modified to enhance its fit with a model of the invention. Such techniques are known as “structure-based ligand design” (See Kuntz et al., 1994, Acc. Chem. Res. 27:117; Guida, 1994, Current Opinion in Struc. Biol. 4: 777; and Colman, 1994, Current Opinion in Struc. Biol. 4: 868, for reviews of structure-based drug design and identification;and Kuntz et al 1982, J. Mol. Biol. 162:269; Kuntz et al., 1994, Acc. Chem. Res. 27: 117; Meng et al., 1992, J. Compt Chem. 13: 505; Bohm, 1994, J. Comp. Aided Molec. Design 8: 623 for methods of structure-based modulator design).

Examples of computer programs that may be used for structure-based ligand design are CAVEAT (Bartlett et al., 1989, in “Chemical and Biological Problems in Molecular Recognition”, Roberts, S. M. Ley, S. V.; Campbell, N. M. eds; Royal Society of Chemistry: Cambridge, pp 182-196); FLOG (Miller et al., 1994, J. Comp. Aided Molec. Design 8:153); PRO Modulator (Clark et al., 1995 J. Comp. Aided Molec. Design 9:13); MCSS (Miranker and Karplus, 1991, Proteins: Structure, Function, and Genetics 8:195); and, GRID (Goodford, 1985, J. Med. Chem. 28:849).

The method may comprise the following steps:

(i) docking a model of a test compound with a model of a selected ligand binding domain;

(ii) identifying one or more groups on the test compound which may be modified to improve their fit in the selected ligand binding domain;

(iii) replacing one or more identified groups to produce a modified test compound model; and

(iv) docking the modified test compound model with the model of the selected ligand binding domain.

Evaluation of fit may comprise the following steps:

(a) mapping chemical features of a test compound such as by hydrogen bond donors or acceptors, hydrophobic/lipophilic sites, positively ionizable sites, or negatively ionizable sites; and

(b) adding geometric constraints to selected mapped features.

The fit of the modified test compound may then be evaluated using the same criteria.

The chemical modification of a group may either enhance or reduce hydrogen bonding interaction, charge interaction, hydrophobic interaction, Van Der Waals interaction or dipole interaction between the test compound and the key amino acid residue(s) of the selected site. Preferably the group modifications involve the addition, removal, or replacement of substituents onto the test compound such that the substituents are positioned to collide or to bind preferentially with one or more amino acid residues that correspond to the key amino acid residues of the selected site.

Identified groups in a test compound may be substituted with, for example, alkyl, alkoxy, hydroxyl, aryl, cycloatkyl, alkenyl, alkynyl, thiol, thioalkyl, thioaryl, amino, or halo groups. Generally, initial substitutions are conservative, i.e., the replacement group will have approximately the same size, shape, hydrophobicity and charge as the original group. It should, of course, be understood that components known in the art to alter conformation should be avoided.

If a modified test compound model has an improved fit, then it may bind to the selected site and be considered to be a “ligand”. Rational modification of groups may be made with the aid of libraries of molecular fragments which may be screened for their capacity to fit into the available space and to interact with the appropriate atoms. Databases of computer representations of libraries of chemical groups are available commercially, for this purpose.

A test compound may also be modified “in situ” (i.e. once docked into the potential binding domain), enabling immediate evaluation of the effect of replacing selected groups. The computer representation of the test compound may be modified by deleting a chemical group or groups, replacing chemical groups, or by adding a chemical group or groups. After each modification to a compound, the atoms of the modified compound and potential binding site can be shifted in conformation and the distance between the modulator and the active site atoms may be scored on the basis of geometric fit and favourable complementary interactions between the molecules. This technique is described in detail in Molecular Simulations User Manual, 1995 in LUDI.

Examples of ligand building and/or searching computer programs include programs in the Molecular Simulations Package (Catalyst), ISIS/HOST, ISIS/BASE, and ISIS/DRAW (Molecular Designs Limited), and UNITY (Tripos Associates).

The “starting point” for rational ligand design may be a known ligand for the enzyme. For example, in order to identify potential modulators of a glycosyltransferase, a logical approach would be to start with a known ligand (for example a substrate molecule or inhibitor ) to produce a molecule which mimics the binding of the ligand. Such a molecule may, for example, act as a competitive inhibitor for the true ligand, or may bind so strongly that the interaction (and inhibition) is effectively irreversible. Such a method may comprise the following steps:

(i) generating a computer model of a glycosyltransferase ligand binding domain in complex with a ligand;

(ii) replacing one or more groups on the ligand computer model to produce a modified ligand; and

(iii) evaluating the fit of the modified ligand in the ligand binding domain.

The replacement groups could be selected and replaced using a compound construction program which replaces computer representations of chemical groups with groups from a computer database, where the representations of the compounds are defined by structural coordinates.

In an embodiment, a screening method is provided for identifying a ligand of a glycosyltransferase comprising the step of using die structural coordinates or model of a substrate molecule or component thereof, defined in relation to its spatial association with a glycosyltransferase structure or a ligand binding domain, to generate a compound that is capable of associating with the glycosyltransferase or ligand binding domain.

The screening methods of the present invention may be used to identify compounds or entities that associate with a molecule that associates with a glycosyltransferase enzyme (for example, a substrate molecule).

Compounds and entities (e.g. ligands) of a glycosyltransferase identified using the above-described methods may be prepared using methods described in standard reference sources utilized by those skilled in the art. For example, organic compounds may be prepared by organic synthetic methods described in references such as March, 1994, Advanced Organic Chemistry: Reactions, Mechanisms, and Structure, New York, McGraw Hill.

Test compounds and ligands which are identified using a model of the present invention can be screened in assays such as those well known in the art. Screening can be, for example, in vitro, in cell culture, and/or in vivo. Biological screening assays preferably centre on activity-based response models, binding assays (which measure how well a compound binds), and bacterial, yeast and animal cell lines (which measure the biological effect of a compound in a cell). The assays can be automated for high capacity-high throughput screening (HTS) in which large numbers of compounds can be tested to identify compounds with the desired activity. The biological assay, may also be an assay for the ligand binding activity of a compound that selectively binds to the ligand binding domain compared to other enzymes.

The invention contemplates a method for the design of modulators for a glycosyltransferase based on a structure or model of a sugar nucleotide donor (or parts thereof) or an acceptor defied in related to its association with a ligand binding domain.

In accordance with particular aspects of the invention, a method is provided for designing potential inhibitors of a glycosyltransferase, preferably GnT I, GnT V, and/or Core 2L GnT, comprising the step of using one or more (preferably all) of the structural coordinates of uracil, uridine, ribose, pyrophosphate, or UDP of Tables 14, 15 or 16, as follows:

Table 14 for GnT1 Ground State

Table 15 for GntV

Table 16 for core 2L

to generate a compound for associating with a ligand binding domain of a glycosyltransferase that associates with uracil, uridine, ribose, pyrophosphate, or UDP.

To generate a compound for associating with the active site of a glycosyltransferase, the following steps are employed in a particular method of the invention: (a) generating a computer representation of uracil, uridine, or UDP defined by structural coordinates of Tables 14, 15 or 16; (b) searching for molecules in a data base that are structurally or chemically similar to the defined uracil, uridine, or UDP using a searching computer program, or replacing portions of the compound with similar chemical structures from a database using a compound-building computer program.

In another embodiment of the invention, a method is provided for designing potential inhibitors of a glycosyltransferase preferably GnT I, GnT V,. and/or Core 2L GnT, said method comprising the step of using one or more (preferably all) of the structural coordinates of UDP-GlcNAc of Tables 17, 18, or 19 as follows:

Table 17 for GnTI transition state

Table 18 for GnTV

Table 19 for core 2L transition state

to generate a compound for associating with a ligand binding domain of a glycosyltransferase that associates with UDP-GlcNAc.

The following steps are employed in a particular method of the invention: (a) generating a computer representation of UDP-GlcNAc defined by one or more (preferably all) of the structural coordinates of Table 17, 18, or 19 appropriate for a specific glycosyltransferase; (b) searching for molecules in a data base that are structurally or chemically similar to the defined UDP-GlcNAc using a searching computer program, or replacing portions of the compound with similar chemical structures from a database using a compound building computer program.

In another embodiment of the invention, a method is provided for designing potential inhibitors of GnT I, said method comprising the step of using one or more (preferably all) of the structural coordinates of Table 20 for an oligosaccharide acceptor, to generate a compound for associating with a ligand binding domain of a glycosyltransferase that associates with the acceptor.

The following steps are employed in a particular method of the invention: (a) generating a computer representation of an oligosaccharide acceptor defined by the one or more (preferably all) of the structural coordinates of Table 20 appropriate for a specific glycosyltransferase; (b) searching for molecules in a data base that are structurally or chemically similar to the defined oligosaccharide acceptor using a searching computer program, or replacing portions of the compound with similar chemical structures from a database using a compound building computer program.

Potential modulators of glycosyltransferases identified using the above-described methods may be prepared using methods described in standard reference sources utilized by those skilled in the art. For example, organic compounds may be prepared by organic synthetic methods described in references such as March, 1994, Advanced Organic Chemistry: Reactions, Mechanisms, and Structure, New York, McGraw Hill.

### Ligands/Modulators

The present invention provides a ligand or compound or entity identified by a screening method of the present invention. A ligand or compound may have been designed rationally by using a model according to the present invention. A ligand or compound identified using the screening methods of the invention specifically associate with a target compound. In the present invention the target compound may be a glycosyltransferase or a molecule that is capable of associating with a glycosyltransferase (for example a ligand or substrate molecule). In a preferred embodiment the ligand is capable of binding to the ligand binding domain of a glycosyltransferase.

A ligand or compound identified using a screening method of the invention may act as a “modulator”, i.e. a compound which affects the activity of a glycosyltransferase. A modulator may reduce, enhance or alter the biological function of a glycosyltransferase. For example a modulator may modulate the capacity of the enzyme to transfer a sugar from a nucleotide sugar donor to a specific hydroxyl of various saccharide acceptors that leads to the formation of a new glycosidic linkage. An alteration in biological function may be characterised by a change in specificity. For example, a modulator may cause the enzyme to accept a different substrate molecule, to transfer a different sugar, or to work with a different metal cofactor. In order to exert its function, a modulator commonly binds to a ligand binding domain.

A modulator which is capable of reducing the biological function of the enzyme may also be known as an inhibitor. Preferably an inhibitor reduces or blocks the capacity of the enzyme to form new glycosidic linkages. The inhibitor may mimic the binding of a substrate molecule, for example, it may be a substrate analogue. A substrate analogue may be designed by considering the interactions between the substrate molecule and the enzyme (for example by using information derivable from a model of the invention) and specifically altering one or more groups.

In a highly preferred embodiment, a modulator acts as an inhibitor of a glycosyltransferase and is capable of inhibiting N- or O-glycan biosynthesis.

The present invention also provides a method for modulating the activity of a glycosyltransferase within a cell using a modulator according to the present invention. It would be possible to monitor the expression of N-glycans on the cell surface following such treatment by a number of methods known in the art (for example by detecting expression with an N-and O-glycan specific antibody).

In another preferred embodiment, the modulator modulates the catalytic mechanism of a glycosyltransferase.

A modulator may be an agonist, partial agonist, partial inverse agonist or antagonist of a glycosyltransferase.

The term “agonist” includes any ligand, which is capable of binding to a glycosyltransferse or ligand binding domain thereof, and which is capable of increasing a proportion of active enzyme, resulting in an increased biological response. The term includes partial agonists and inverse agonists.

The term “partial agonist” includes an agonist that is unable to evoke the maximal response of a biological system, even at a concentration sufficient to saturate a specific glycosyltransferase or ligand binding domain thereof.

The term “partial inverse agonist” includes an inverse agonist that evokes a submaximal response to a biological system, even at a concentration sufficient to saturate the specific receptors. At high concentrations, it will diminish the actions of a full inverse agonist.

The invention relates to a glycosyltransferase ligand binding domain antagonist, wherein said ligand binding domain is that defined by the amino acid structural coordinates described herein. For example the ligand may antagonise the inhibition of glycosyltransferase by an inhibitor.

The term “antagonist” includes any agent that reduces the action of another agent, such as an agonist. The antagonist may act at the same site as the agonist (competitive antagonism). The antagonistic action may result from a combination of the substance being antagonised (chemical antagonism) or the production of an opposite effect through a different molecule (functional antagonism or physiological antagonism) or as a consequence of competition for the binding site of an intermediate that links the enzyme to the effect observed (indirect antagonism).

The term “competitive antagonism” refers to the competition between an agonist and an antagonist for a glycosyltransferase or ligand binding domain thereof that occurs when the binding of agonist and antagonist becomes mutually exclusive. This may be because the agonist and antagonist compete for the same binding site or combine with adjacent but overlapping sites. A third possibility is that different sites are involved but that they influence the glycosyltransferase or ligand binding domain in such a way that agonist and antagonist molecules cannot be bound at the same time. If the agonist and antagonist form only short lived combinations with a glycosyltransferase or ligand binding domain thereof so that equilibrium between agonist, antagonist and glycosyltransferase and ligand binding domain thereof is reached during the presence of the agonist, the antagonism will be surmountable over a wide range of concentrations. In contrast, some antagonists, when in close enough proximity to their binding site, may form a stable covalent bond with it and the antagonism becomes insurmountable when no spare ligand binding domain remains.

As mentioned above, an identified ligand or compound may act as a ligand model (for example, a template) for the development of other compounds. A modulator may be a mimetic of a ligand or ligand binding domain. A mimetic of a ligand may compete with a natural ligand for a glycosyltransferase or ligand binding domain thereof, and antagonize a physiological effect of the enzyme in an animal. A mimetic of a ligand may be an organically synthesized compound. A mimetic of a ligand binding domain, may be either a peptide or other biopharmaceutical (such as an organically synthesized compound) that specifically binds to a natural substrate molecule for a glycosyltransferase and antagonize a physiological effect of the enzyme in an animal.

Once a ligand has been optimally selected or designed, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to a glycosyltransferase ligand binding domain by the same computer methods described above.

Preferably, positions for substitution are selected based on the predicted binding orientation of a ligand to a glycosyltransferase ligand binding domain.

A technique suitable for preparing a modulator will depend on its chemical nature. For example, organic compounds may be prepared by organic synthetic methods described in references such as March, 1994, Advanced Organic Chemistry: Reactions, Mechanisms, and Structure, New York, McGraw Hill. Peptides can be synthesized by solid phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Once cleaved from the resin, the peptide may be purified by preparative high performance liquid chromatography (e.g., Creighton (1983) Proteins Structures and Molecular Principles, W H Freeman and Co, New York N.Y.). The composition of the synthetic peptides may be confirmed by amino acid analysis or sequencing (e.g., the Edman degradation procedure; Creighton, supra).

If a modulator is a nucleotide, or a polypeptide expressable therefrom, it may be synthesized, in whole or in part, using chemical methods well known in the art (see Caruthers M H et al (1980) Nuc Acids Res Symp Ser 215-23, Horn T et al (1980) Nuc Acids Res Symp Ser 225-232), or it may be prepared using recombinant techniques well known in the art.

Direct synthesis of a ligand or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences obtainable from the ligand, or any part thereof, may be altered during direct synthesis and/or combined using chemical methods with a sequence from other subunits, or any part thereof, to produce a variant ligand.

In an alternative embodiment of the invention, the coding sequence of a ligand or mimetics thereof may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

A wide variety of host cells can be employed for expression of the nucleotide sequences encoding a ligand of the present invention. These cells may be both prokaryotic and eukaryotic host cells. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the expression products to produce an appropriate mature polypeptide. Processing includes but is not limited to glycosylation, ubiquitination, disulfide bond formation and general post-translational modification.

In an embodiment of the present invention, the ligand may be a derivative of, or a chemically modified ligand. The term “derivative” or “derivatised” as used herein includes the chemical modification of a ligand.

A chemical modification of a ligand and/or a key amino acid residue of a ligand binding domain of the present invention may either enhance or reduce hydrogen bonding interaction, charge interaction, hydrophobic interaction, Van Der Waals interaction or dipole interaction between the ligand and the key amino acid residue(s) of a glycosyltransferase ligand binding domain.

Preferably such modifications involve the addition of substituents onto a test compound such that the substituents are positioned to collide or to bind preferentially with one or more amino acid residues that correspond to the key amino acid residues of a glycosyltransferase ligand binding domain. Typical modifications may include, for example, the replacement of a hydrogen by a halo group, an alkyl group, an acyl group or an amino group.

The invention also relates to classes of modulators of a glycosyltransferase based on the structure and shape of a substrate, defined in relation to the substrate molecule's spatial association with a glycosyltransferase structure of the invention or part thereof. Therefore, a modulator may comprise a substrate molecule having the shape or structure, preferably the structural coordinates, of a substrate molecule in an active site or ligand binding domain of a reaction catalyzed by a glycosyltransferase.

### Modulators Based on the 3D Structure of a Nucleotide Sugar Donor

One class of modulators (i.e. inhibitors) of a glycosyltransferase, preferably GnT I, GnT V, and/or Core 2L GnT, comprise the structure of uracil, uridine, ribose, pyrophosphate, or UDP with one or more (preferably all) of the structural coordinates of uracil, uridine, ribose, pyrophosphate, or UDP of Tables 14, 15 or 16 as follows:

Table 14 for Gnt1 Ground State

Table 15 for GnTV

Table 16 for core 2L

In an embodiment, the invention provides inhibitors of a glycosyltransferase, preferably GnT I, GnT V. and/or Core 2L GnT, comprising the structure of UDP-GlcNAc and having one or more (preferably all) of the structural coordinates of UDP-GlcNAc of Tables 17, 18, or 19 as follows:

Table 17 for GnT1 transition state

Table 18 for GnTV,

Table 19 for core 2L

Another class of modulators defined by the invention are compounds of the Formula I having the structural coordinates of uracil of Table 14, 15 or 16, preferably the first conformation in each Table:

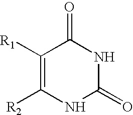

wherein R₁ and R₂ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, pyrophophate, gallic acid, phosphonates, thioamide, and —OR₁₀ where R₁₀ is alkyl, cycloalkyl, alkenyl, alkynyl, or heterocyclic ring;

and salts and optically active and racemic forms of a compound of the formula I.

Yet another class of modulators defined by the invention are compounds of the formula II having the structural coordinates of uridine of Table 14, 15, or 16, preferably, the first conformation in each Table:

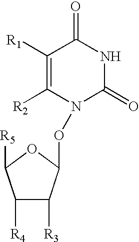

wherein R₁, R₂, R₃, R4, and R₅ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, pyrophosphate, gallic acid, phosphonates, thioamide, and —OR₁₀ where R₁₀ is alkyl, cycloalkyl, alkenyl, alkynyl, or heterocyclic ring,

and salts and optically active and racemic forms of a compound of the formula II.

Yet another class of modulators identified by the invention are compounds of the formula III having the structural coordinates of UDP of Tables 14, 15, or 16, preferably the first conformation in each Table:

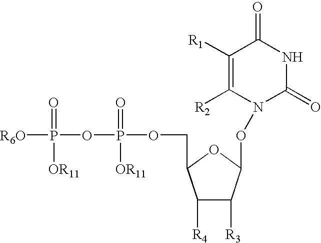

wherein R₁, R₂, R₃, R, R₅, and R₆ are each independently hydrogen, alkyl, cycloalklyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, gallic acid, phosphonates, thioamide, and —OR₁₀ where R₁₀ is alkyl, cycloalkyl, alkenyl, alkynyl, or heterocyclic ring, R₆ may be a monosaccharide or disaccharide, preferably a monosaccharide, including GlcNAc, glucose, and mannose, and salts and optically active and racemic forms of a compound of the formula III.

Yet another class of modulators are compounds of the formula IV having the structural coordinates of UDP-GlcNAc of Table 17, 18, or 19, preferably the first conformation in each Table:

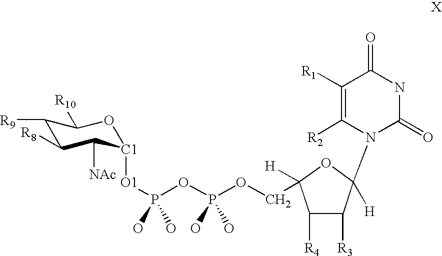

wherein R₁, R₂, R₃, R₄, and R₅ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, gallic acid, phosphonates, thioamide, and —OR₁₀ where R₁₀ is alkyl, cycloalkyl, alkenyl, alkynyl, or heterocyclic ring,

and salts and optically active and racemic forms of a compound of the formula IV.

One or more of R₁, R₂, R₃, R4, R₅, and/or R6, alone or together, which contain available functional groups as described herein, may be substituted with for example one or more of the following: alkyl, alkoxy, hydroxyl, aryl, cycloalkyl, alkenyl, alkynyl, thiol, thioalkyl, thioaryl, amino, or halo. The term “one or more” used herein preferably refers to from 1 to 2 substituents.

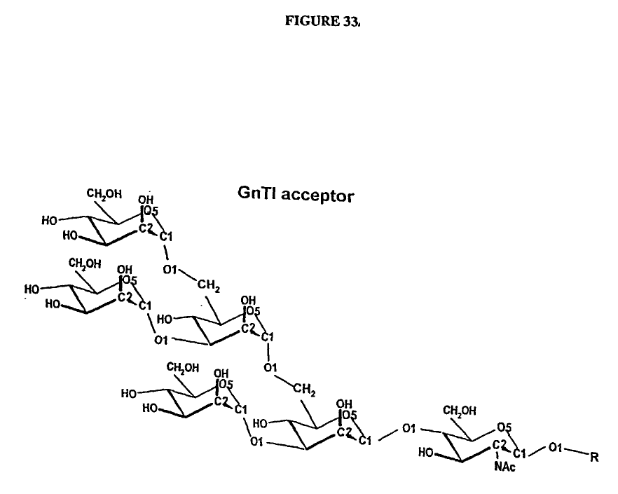

Modulators (e.g. inhibitors) are also contemplated that have the structure of an acceptor of a glycosyltransferase, and are characterized by the structural coordinates of an acceptor for a glycosyltransferase of Table 20. The acceptor may have the structure as shown in FIG. 19A or 33. Functional groups in the acceptor structure may be substituted with for example, alkyl, alkoxy, hydroxyl, aryl, cycloalkyl, alkenyl, alkynyl, thiol, thioalkyl, thioaryl, amino, or halo, or they may be modified using techniques known in the art.

### Modulators Derived From the Transition State Sugar Binding Cavity

A class of modulators defined by the invention are compounds comprising the structural coordinates of GlcNAc in the transition state of a reaction catalyzed by a glycosyltransferase, preferably Core 2 GnT-L and GnT-I. The GlcNAc has a half chair or distorted chair conformation, a partial double bond between C1 and 05, and a hybridization Sp² at C1.

Yet another class of modulators defined by the invention are compounds comprising a pyrophosphate group directly or indirectly linked to GlcNAc having the structural coordinates of GlcNAc in the transition state of a reaction catalyzed by a glycosyltransferase, preferably Core 2 GnT-L and GnT-I. The GlcNAc component has a half chair or distorted chair conformation, a partial double bond between Cl and 05, and a hybridization Sp2 at C1. The distance between the pyrophosphate group and the GlcNAc is about 1.9 to 3.5 Å, preferably 2.2 to 3.0 Å.

The compounds may comprise analogues and derivatives of GlcNAc or the pyrophosphate group. For example, reactive groups of the GlcNAc or pyrophosphate group may be modified or they may be substituted with alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof (e.g. —CH₂OH), amines, sulfate, sulfonic or sulfinic acid or esters thereof, phosphate, gallic acid, phosphonates, thioamide, and —OR₁₂ where R₁₂ is alkyl, cycloalkyl, alkenyl, alkynyl, or a heterocyclic ring. The GlcNAc and pyrophosphate may be linked via any molecules suitable for linking a sugar and phosphate group.

The present invention contemplates all optical isomers and racemic forms thereof of the compounds (modulators) of the invention described herein, and the formulas of the compounds shown herein are intended to encompass all possible optical isomers of the compounds so depicted.

The present invention also contemplates salts and esters of the compounds (modulators) of the invention described herein. In particular, the present invention includes pharmaceutically acceptable salts. By pharmaceutically acceptable salts is meant those salts which are suitable for use in contact with the tissues of humans and lower animals without undue toxicity, irritation, allergic response and the like, and are commensurate with a reasonable benefit/risk ratio. Pharmaceutically acceptable salts are well known in the art and are described for example, in S. M. Berge, et al., J. Pharmaceutical Sciences, 1977, 66:1-19.

### Peptide Modulators Derived From the Loop Structure of a Glycosyltransferase

The invention provides peptides that are derived from the loop structure of a glycosyltransferase. For example, peptides of the invention include the amino acids EER, HVNT, or VSHG that bind to a pyrophosphate group of a sugar nucleotide donor. Other proteins containing these binding domain sequences may be identified with a protein homology search, for example by searching available databases such as GenBank or SwissProt and various search algorithms and/or programs may be used including FASTA, BLAST (available as a part of the GCG sequence analysis package, University of Wisconsin, Madison, Wis.), or ENTREZ (National Center for Biotechnology Information, National Library of Medicine, National Institutes of Health, Bethesda, Md.).

In accordance with an embodiment of the invention, specific peptides are contemplated that mediate the association of the loop structure of a Core 2 transferase and a pyrophosphate group of a sugar nucleotide donor for the Core 2 transferase. In particular, a peptide of the following formula is provided which interferes with the association of the loop structure of a Core 2 transferase and a pyrophosphate group of a sugar nucleotide donor for the Core 2 transferase:

X-X¹X²-X³-X⁴

wherein X represents 0 to 70, preferably 0 to 50 amino acids, more preferably 2 to 20 amino acids, X¹ and X² independently represent an amino acid with a charged polar group, preferably Glu, Asp, Asn, or Gin, X³ represents a basic amino acid, preferably Arg, His, or Lys, and X⁴ represents 0 to 70, preferably 0 to 50 amino acids, more preferably 2 to 20 amino acids. In specific embodiments, X¹ and X² are Glu, and X³ is Arg.

In an embodiment of the present invention a peptide is provided where X represents X⁵—SHK where X⁵ represents 0 to 70, preferably 0 to 50 amino acids, more preferably 2 to 20 amino acids, or X⁴ represents X⁶-NRKRYE where X⁶ represents 0 to 70, preferably 0 to 50 amino acids, more preferably 2 to 20 amino acids

Preferred peptides of the invention include SHKEERNRKRYE, SHKDERNRKRYE, SHKEDRNRNYE, SHKEENNRKRYE, SHKDDRNRKRYE, and SHKNERNRKRYE.

In accordance with another embodiment of the invention, specific peptides are contemplated that mediate the association of the loop structure of a GnT-I to V transferase and a pyrophosphate group of a sugar nucleotide donor for the transferase. In particular, a peptide of the following formula is provided which interferes with the association of the loop structure of a GnT-I to V transferase and a pyrophosphate group of a sugar nucleotide donor for the transferase:

X⁷-X⁸-X⁹-X¹⁰-X¹¹-X¹²

wherein X⁷ represents 0 to 70, preferably 0 to 50 amino acids, more preferably 2 to 20 amino acids, X8 represents Val or His, X⁹ represents Val or Ser, X¹⁰ represents Asn, or His, X¹¹ represents Thr or Gly, and X¹² represents 0 to 70, preferably 0 to 50 amino acids, more preferably 2 to 20 amino acids.

In an embodiment of the present invention a peptide is provided where X⁷ represents X¹³-FIGRP or X¹³-GRKG where X¹³ represents 0 to 70, preferably 0 to 50 amino acids, more preferably 2 to 20 amino acids or X¹² represents X¹⁴-DLN or X¹⁴—QFF, where X¹⁴ represents 0 to 70, preferably 0 to 50 amino acids, 20 amino acids.

Preferred peptides of the invention include FIGRPHVNTDLN, and GRKGVSHGQFF.

All of the peptides of the invention, as well as molecules substantially homologous, complementary or otherwise functionally or structurally equivalent to these peptides may be used for purposes of the present invention. In addition to full-length peptides of the invention, truncations of the peptides are contemplated in the present invention. Truncated peptides may comprise peptides of about 7 to 10 amino acid residues

The truncated peptides may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated peptides may have a carboxyl group, an amido group, a T-butyloxycarbonyl group, or a macromolecule including but not limited to lipid-fatty acid conjugates, polyethylene glycol, or carbohydrates at the carboxy terminal end.

The peptides of the invention may also include analogs of a peptide of the invention and/or truncations of a peptide, which may include, but are not limited to a peptide of the invention containing one or more amino acid insertions, additions, or deletions, or both. Analogs of a peptide of the invention exhibit the activity characteristic of a peptide e.g. interference with the interaction of a loop structure of a glycosyltransferase and a pyrophosphate of a sugar nucleotide donor, and may further possess additional advantageous features such as increased bioavailability, stability, or reduced host immune recognition. One or more amino acid insertions may be introduced into a peptide of the invention. Amino acid insertions may consist of a single amino acid residue or sequential amino acids.

One or more amino acids, preferably one to five amino acids, may be added to the right or left termini of a peptide of the invention. Deletions may consist of the removal of one or more amino acids, or discrete portions from the peptide sequence. The deleted amino acids may or may not be contiguous. The lower limit length of the resulting analog with a deletion mutation is about 7 amino acids.

It is anticipated that if amino acids are inserted or deleted in sequences outside an X¹-X²-X³ or X⁸-X ⁹-X¹⁰-X¹¹ sequence that the resulting analog of the peptide will exhibit the activity of a peptide of the invention.

The invention also includes a peptide conjugated with a selected protein, or a selectable marker (see below) to produce fusion proteins.

The peptides of the invention may be prepared using recombinant DNA methods. Accordingly, nucleic acid molecules which encode a peptide of the invention may be incorporated in a known manner into an appropriate expression vector which ensures good expression of the peptide. Possible expression vectors include but are not limited to cosmids, plasmids, or modified viruses so long as the vector is compatible with the host cell used. The expression vectors contain a nucleic acid molecule encoding a peptide of the invention and the necessary regulatory sequences for the transcription and translation of the inserted protein-sequence. Suitable regulatory sequences may be obtained from a variety of sources, including bacterial, fungal, viral, mammalian, or insect genes. (For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Other sequences, such as an origin of replication, additional DNA restriction sites, enhancers, and sequences conferring inducibility of transcription may also be incorporated into the expression vector.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest encoding a peptide of the invention.

The recombinant expression vectors may also contain genes that encode a fusion portion which provides increased expression of the recombinant peptide; increased solubility of the recombinant peptide; and/or aid in the purification of the recombinant peptide by acting as a ligand in affinity purification. For example, a proteolytic cleavage site may be inserted in the recombinant peptide to allow separation of the recombinant peptide from the fusion portion after purification of the fusion protein. Examples of fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors may be introduced into host cells to produce a transformant host cell. Transformant host cells include prokaryotic and eukaryotic cells which have been transformed or transfected with a recombinant expression vector of the invention. The terms “transformed with”, “transfected with”, “transformation” and “transfection” are intended to include the introduction of nucleic acid (e.g. a vector) into a cell by one of many techniques known in the art. For example, prokaryotic cells can be transformed with nucleic acid by electroporation or calcium-chloride mediated transformation. Nucleic acid can be introduced into mammalian cells using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, the peptides of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

The peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, ₂nd Ed., Pierce Chemical Co., Rockford Ill. (1984) and G. Barany and R B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles fo Peptide Synthesis, Springer-Verlag, Berlin 1984, and E. Gross and J. Meienhofer, Eds., The Peptides: Analysis, Synthesis, Biologu, suprs, Vol 1, for classical solution synthesis).

N-terminal or C-terminal fusion proteins comprising a peptide of the invention conjugated with other molecules may be prepared by fusing, through recombinant techniques, the N-terminal or C-terminal of the peptide, and the sequence of a selected protein or selectable marker with a desired biological function. The resultant fusion proteins contain the peptide fused to the selected protein or marker protein as described herein. Examples of proteins which may be used to prepare fusion proteins include immunoglobulins, glutathione-S-transferase (GST), hemagglutinin (HA), and truncated myc.

Cyclic derivatives of the peptides of the invention are also part of the present invention. Cyclization may allow the peptide to assume a more favorable conformation for association with a ligand binding domain. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The side chains of Tyr and Asn may be linked to form cyclic peptides. The components that form the bonds may be side chains of amino acids, non-amino acid components or a combination of the two. In aspects of the invention, cyclic peptides may have a beta-turn in the right position. Beta-turns may be introduced into the peptides of the invention by adding the amino acids Pro-Gly at the right position.

It may be desirable to produce a cyclic peptide that is more flexible than the cyclic peptides containing peptide bond linkages as described above. A more flexible peptide may be prepared by introducing cysteines at the right and left position of the peptide and forming a disulphide bridge between the two cysteines. The peptide is more flexible as a result of the length of the disulfide linkage and the smaller number of hydrogen bonds in the beta-sheet portion. The relative flexibility of a cyclic peptide can be determined by molecular dynamics simulations.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Combined with certain formulations, peptides can be effective intracellular agents. However, in order to increase the efficacy of peptides, a fusion peptide can be prepared comprising a second peptide which promotes “transcytosis”, e.g. uptake of the peptide by epithelial cells. To illustrate, a peptide of the invention can be provided as part of a fusion polypeptide with all or a fragment of the N-terminal domain of the HIV protein Tat, e.g. residues 1-72 of Tat or a smaller fragment thereof which can promote transcytosis. In other embodiments, a peptide of the invention can be provided as a fusion polypeptide with all or a portion of an antennapedia protein. To further illustrate, a peptide of the invention can be provided as a chimeric peptide which includes a heterologous peptide sequence (“internalizing peptide”) which drives the translocation of an extracellular form of a peptide sequence across a cell membrane in order to facilitate intracellular localization of the peptide.

The peptides may be developed using a biological expression system. The use of these systems allows the production of large libraries of random peptide sequences and the screening of these libraries for peptide sequences that bind to the loop structure. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

Peptides of the invention may be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds which can be tested for predicted properties as related to the target molecule (i.e. glycosyltranaferases or ligand binding domain thereof). The activity of the lead compounds can be evaluated using, assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess desired activities.

The peptides of the invention may be used to prepare antibodies. Conventional methods can be used to prepare the antibodies.

The peptides and antibodies specific for the peptides of the invention may be labelled using conventional methods with various enzymes, fluorescent materials, luminescent materials and radioactive materials. Suitable enzymes, fluorescent materials, luminescent materials, and radioactive material are well known to the skilled artisan. Antibodies and labeled antibodies specific for the peptides of the invention may be used to screen for proteins containing loop structures or they may be used to modulate the activity of a glycosyltransferase.

Computer modelling techniques known in the art may also be used to observe the interaction of a peptide of the invention, and truncations and analogs thereof with a pyrophosphate of a sugar nucleotide donor (for example, Homology Insight 11 and Discovery available from BioSym/Molecular Simulations, San Diego, Calif., U.S.A.). If computer modelling indicates a strong interaction, the peptide can be synthesized and tested for its ability to interfere with the binding of the molecules of a complex discussed herein.

The present invention also contemplates salts and esters of the peptides of the invention. In particular, the present invention includes pharmaceutically acceptable salts. By pharmaceutically acceptable salts is meant those salts which are suitable for use in contact with the tissues of humans and lower animals without undue toxicity, irritation, allergic response and the like, and are commensurate with a reasonable benefit/risk ratio. Pharmaceutically acceptable salts are well known in the art and are described for example, in S. M. Berge, et al., J. Pharmaceutical Sciences, 1977, 66:1-19.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

### Compositions and Methods of Treatment

A secondary, tertiary, or quantemary glycosyltransferase structure or models of the invention and the modulators identified using the methods of the invention may be used to modulate the biological activity of a glycosyltransferase in a cell, including modulating a pathway in a cell regulated by the glycosyltransferase or modulating a glycosyltransferase with inappropriate activity in a cellular organism.

The modulators can be administered for prophylactic and/or therapeutic treatments. In therapeutic applications, compositions are administered to a patient already suffering from a condition, in an amount sufficient to cure or at least alleviate the symptoms of the disease and its complications. In prophylactic applications, modulators are administered to a patient susceptible to or otherwise at risk of a particular condition.

Cellular assays, as well as animal model assays in vivo, may be used to test the activity of a potential modulator of a glycosyltransferase as well as diagnose a disease associated with inappropriate glycosyltransferase activity. In vivo assays are also useful for testing the bioactivity of a potential modulator designed by the methods of the invention.

The modulators (e.g. inhibitors) identified using the methods of the invention can be useful in the treatment and prophylaxis of tumor growth and metastasis of tumors. Anti-metastatic effects of inhibitors can be demonstrated using a lung colonization assay. For example, melanoma cells treated with an inhibitor may be injected into mice and the ability of the melanoma cells to colonize the lungs of the mice may be examined by counting tumor nodules on the lungs after death. Suppression of tumor growth in mice by the inhibitor administered orally or intravenously may be examined by measuring tumor volume.

An inhibitor identified using the invention can have particular application in the prevention of tumor recurrence after surgery i.e. as an adjuvant therapy.

An inhibitor can be especially useful in the treatment of various forms of neoplasia such as leukemias, lymphomas, melanomas, adenomas, sarcomas, and carcinomas of solid tissues in patients. In particular, inhibitors can be used for treating malignant melanoma, pancreatic cancer, cervico-uterine cancer, ovarian cancer, cancer of the kidney such as metastatic renal cell carcinoma, stomach, lung, rectum, breast, bowel, gastric, liver, thyroid, head and neck cancers such as unresectable head and neck cancers, lymphangitis carcinamatosis, cancers of the cervix, breast, salivary gland, leg, tongue, lip, bile duct, pelvis, mediastinum, urethra, bronchogenic, bladder, esophagus and colon, non-small cell lung cancer, and Kaposi's Sarcoma which is a form of cancer associated with HIV-infected patients with Acquired Immune Deficiency Syndrome (AIDS). The inhibitors may also be used for other anti-proliferative conditions such as bacterial and viral infections, in particular AIDS.

An inhibitor identified in accordance with the present invention can be used to treat immunocompromised subjects. For example, they can be used in a subject infected with HIV, or other viruses or infectious agents including bacteria, fungi, and parasites, in a subject undergoing bone marrow transplants, and in subjects with chemical or tumor-induced immune suppression.

Inhibitors may be used as hemorestorative agents and in particular to stimulate bone marrow cell proliferation, in particular following chemotherapy or radiotherapy. The myeloproliferative activity of an inhibitor of the invention may be determined by injecting the inhibitor into mice, sacrificing the mice, removing bone marrow cells and measuring the ability of the inhibitor to stimulate bone marrow proliferation by directly counting bone marrow cells and by measuring clonogenic progenitor cells in methylcellulose assays. The inhibitors can also be used as chemoprotectants and in particular to protect mucosal epithelium following chemotherapy.

An inhibitor identified in accordance with the invention also may be used as an antiviral agent in particular on membrane enveloped viruses such as retroviruses, influenza viruses, cytomegaloviruses and herpes viruses. A small molecule inhibitor can also be used to treat bacterial, fungal, and parasitic infections. For example, a small molecule inhibitor can be used to prevent or treat infections caused by the following: Neisseria species such as Neisseria meningitidis, and N. gonorrheae; Chlamydia species such as Chlamydia pneumoniae, Chlamydia psittaci, Chlamydia trichomatis; Escherichia coli, Haemophilus species such as Haemophilus influenza; Yersinia enterocolitica; Salmonella species such as S. typhimurium; Shigella species such as Shigella flexneri; Streptococcus species such as S. agalactiae and S. pneumoniae; Bacillus species such as Bacillus subtilis; Branhamella catarrhalis; Borrelia burgdorfer; Pseudomonas aeruginosa; Coxiella burnetti; Campylobacter species such as C.hyoilei; Helicobacter pylori; and, Klebsiella species such as Klebsiella pneumoniae.

An inhibitor can also be used in the treatment of inflammatory diseases such as rheumatoid arthritis, asthma, inflammatory bowel disease, and atherosclerosis. In particular, an inhibitor of core 2L may be used in the treatment of inflammatory diseases.

An inhibitor can also be used to augment the anti-cancer effects of agents such as interleukin-2 and poly-IC, to augment natural killer and macrophage tumoricidal activity, induce cytokine synthesis and secretion, enhance expression of LAK and HLA class I specific antigens; activate protein kinase C, stimulate bone marrow cell proliferation including hematopoietic progenitor cell proliferation, and increase engraftment efficiency and colony-forming unit activity, to confer protection against chemotherapy and radiation therapy (e.g. chemoprotective and radioprotective agents), and to accelerate recovery of bone marrow cellularity particularly when used in combination with chemical agents commonly used in the treatment of human diseases including cancer and acquired immune deficiency syndrome (AIDS). For example, an inhibitor can be used as a chemoprotectant in combination with anti-cancer agents including doxorubicin, 5-fluorouracil, cyclophosphamide, and methotrexate, and in combination with isoniazid or NSAID.

The present invention thus provides a method for treating the above-mentioned conditions in a subject comprising administering to a subject an effective amount of a modulator identified using the methods of the invention. The invention also contemplates a method for stimulating or inhibiting tumor growth or metastasis in a subject comprising administering to a subject an effective amount of a modulator identified using the methods of the invention. The invention further contemplates the use of a modulator to treat the above-mentioned conditions in a subject.

The invention still further relates to a pharmaceutical composition which comprises a glycosyltransferase structure of the invention or a ligand binding domain thereof (e.g. a loop structure, a hydrophobic pocket), or a modulator identified using the methods of the invention in an amount effective to regulate one or more of the above-mentioned conditions (e.g. tumor growth or metastasis) and a pharmaceutically acceptable carrier, diluent or excipient. The invention contemplates the use of a modulator in the preparation of a pharmaceutical composition.

The compositions of the invention are administered to subjects in a biologically compatible form suitable for pharmaceutical administration in vivo. By “biologically compatible form suitable for administration in vivo” is meant a form of the protein to be administered in which any toxic effects are outweighed by the therapeutic effects of the protein. The terms “subject” or “individual” are intended to include mammals and includes humans, dogs, cats, mice, rats, and transgenic species thereof. Administration of a therapeutically active amount of the pharmaceutical compositions of the present invention is defined as an amount effective, at dosages and for periods of time necessary to achieve the desired result. For example, a therapeutically active amount of a three dimensional glycosyltransferase structure of the invention or modulators of the invention may vary according to factors such as the condition, age, sex, and weight of the individual. Dosage regimes may be adjusted to provide the optimum therapeutic response. For example, several divided doses may be administered daily or the dose may be proportionally reduced as indicated by the exigencies of the therapeutic situation.

The active compound may be administered in a convenient manner such as by injection (subcutaneous, intravenous, etc.), oral administration, inhalation, transdermal application, or intracerebral administration.

A pharmaceutical composition of the invention can be administered to a subject in an appropriate carrier or diluent, co-administered with enzyme inhibitors or in an appropriate carrier such as microporous or solid beads or liposomes. The term “pharmaceutically acceptable carrier” as used herein is intended to include diluents such as saline and aqueous buffer solutions. Liposomes include water-in-oil-in-water emulsions as well as conventional liposomes (Strejan et al., (1984) J. Neuroimmunol 7:27). The active compound may also be administered parenterally or intraperitoneally. Dispersions can also be prepared in glycerol, liquid polyethylene glycols, and mixtures thereof and in oils. Under ordinary conditions of storage and use, these preparations may contain a preservative to prevent the growth of microorganisms. Depending on the route of administration, the active compound may be coated to protect the compound from the action of enzymes, acids, and other natural conditions which may inactivate the compound.

Therapeutic administration of polypeptides may also be accomplished using gene therapy. A nucleic acid including a promoter operatively linked to a heterologous polypeptide may be used to produce high-level expression of the polypeptide in cells transfected with the nucleic acid. DNA or isolated nucleic acids may be introduced into cells of a subject by conventional nucleic acid delivery systems. Suitable delivery systems include liposomes, naked DNA, and receptor-mediated delivery systems, and viral vectors such as retroviruses, herpes viruses, and adenoviruses.

The therapeutic efficacy and safety of a modulator or composition of the invention can be determined by standard pharmaceutical procedures in cell cultures or animal models. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

### EXAMPLES

The following non-limiting examples illustrate the invention:

### Example 1

### Model and Computational Procedures

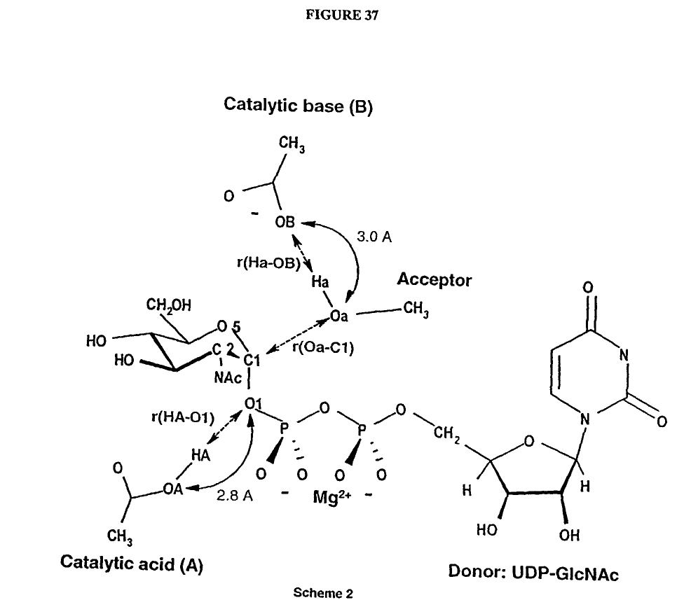

The structural model used in this investigation to analyze the GlcNAc transfer by an N-acetylglucosaminyltransferase enzymatic mechanism computationally consists of all the essential molecules or their fragments, assumed to be involved in the enzymatic mechanism (FIG. 37). The reaction site model contains a complete sugar-donor molecule, UDP-GlcNAc, a hydroxyl group of the oligosaccharide-acceptor modeled by methanol, a divalent metal cofactor modeled by Mg²⁺, as well as the essential parts of the catalytic acid (A) and catalytic base (B) represented by acetic acid and acetate molecules. Such a model of the active site allows all the required electronic rearrangements occurring during the enzymatic reaction such as the proton transfers between the active site components and the substrates. This model consists of 86 atoms and has an overall charge of minus one. In the construction of this model, the relative position of the different participants was an important issue that could not be restricted by the usual means of crystallographic data since no structure of a N-acetylglucosaminyltransferase complexed with the entire UDP-GlcNAc substrate was available. As a consequence, the conformation of the UDP-GlcNAc used in the model is based on previous extensive calculations on sugar-phosphate and diphosphate models (23-25). The two catalytic amino acids present in the model were placed in an arrangement that emulates their orientation in the active site of inverting glycosyl hydrolases (16) where the two carboxylates are located 6.5 Å to 9.5 Å apart. In the model, the two amino acids are located about 5.0 Å away from the anomeric carbon C1. The methanol oxygen atom Oa, representing the reactive hydroxyl of the sugar-acceptor, was initially placed at 3.0 Å from the anomeric carbon C1 and at 3.0 Å from the oxygen OB of the amino acid noted B on FIG. 37. Geometrical constraints applied to fix the relative position of the different components are another important element to consider in building a physically meaningful model. Because the whole structure of the enzyme is not used in the model, these constraints are essential to prevent movement of residues to unrealistic positions with respect to the substrates. The positions of the relevant oxygen atoms of both the catalytic base and catalytic acid have been restricted in the model.

In addition to the nucleophilic attack, the transfer of either one or two protons can be involved in the catalytic reaction of GlcNAc-Ts. Consequently, three distances were used as reaction coordinates in order to follow the mechanism (FIG. 37): the distance r_{HA-O1} between the proton HA of the catalytic acid and the glycosidic oxygen O1; the distance r_{Ha-OB} between the proton Ha of the sugar-acceptor and the oxygen OB of the catalytic base; and the r_{C1-Oa} distance between the anomeric carbon C1 and the oxygen Oa of the acceptor hydroxyl group. These geometrical parameters reflect the proton transfer process from the catalytic acid to the sugar-donor, the proton transfer process from the sugar-acceptor to the catalytic base, and the nucleophilic attack of the sugar-acceptor on the anomeric C1-O1 linkage. The energy of the model calculated as a function of these three reaction-coordinates gives the Potential Energy Surface (PES). Each calculated point on the PES corresponds to the optimized structure and arrangement of the model for the given r_{HA-O1}, r_{Ha-OB}, and r_{C1-Oa} distances. These distances were varied by 0.2 Å increments within the 0.9 Å to 2.1 Å range for r_{Ha-OB}, 0.9 Å to 1.9 Å range for r_{HA-O1} and within the 3.0 Å to 1.3 Å range for r_{C1-Oa}. During the optimization, all geometrical parameters were optimized with exception of the location of two amino acids. As a result, each point on the PES represented by fixed values of the r_{HA-O1}, r_{Ha-OB}, and r_{C1-Oa} distances have all their geometrical variables adjusted to their most stable values. Since, the calculation of such maps requires an extremely large amount of CPU time, the calculation of the PESs was divided into two parts. The first PES corresponds to the energy calculated as a function of the r_{Ha-OB} and r_{C1-Oa} distances whereas for the second PES, the energy was computed as a function of the r_{HA-O1}, and r_{C1-Oa}, distances. The location of the local minima and transition barriers on the PESs is only approximate and for that reason a further optimization of the stationary points with no constraints on the r_{Ha-O1}, r_{Ha-OB}, and r_{C1-Oa} distances is required. These stationary points represent structures of the intermediates and transition states found on the different PESs and along the different reaction pathways. However, in order to avoid any confusion, the same acronyms TSi, and INTi respectively are used herein for the barriers located on PESs and for the stationary points that follow.

The ab initio calculations were carried out with the Jaguar program (26). The optimization of the geometry was performed at the SCF level with the 6-31G* (834 basis functions) basis set. Full optimizations were accomplished using the gradient optimization routines of the program without any symmetry constraints. To better characterize the individual reaction paths, the location and structure of the diverse transition states were calculated using three nearest points to the particular barrier on the PES using the QST-guided search of the Jaguar software (26). The geometry of all stationary points on PESs was then fully optimized using the 6-3 1G* basis set. The effects of electron correlation on the potential energy surface were estimated using the B3LYP density functional method (27). Ultimately, selected geometries were used to estimate the effect of the basis set by calculating their single point energy with the 6-31++G** basis set (1178 basic functions).

### Results and Discussion

The only experimental data available to date on the mechanism of N-acetylglucosaminyltransferase are kinetic studies on GlcNAc-T I and GlcNAc-T II (6, 22). They indicate an ordered sequential mechanism and the prerequisite of a metal cofactor for the enzyme activity. A metal cofactor has been shown to be generally required by these N-acetylglucosaminyltransferases, presumably to increase the leaving ability of the pyrophosphate group. This metal ion binds to the enzyme prior to the donor nucleotide-sugar. Then, before any of the products leaves the enzyme, a sugar-acceptor has to be bound in order to proceed to the transfer of the sugar. For some galactosyltransferases (28), the metal cofactor was found to be released from the enzyme in the form of a complex with the nucleotide-diphosphate.

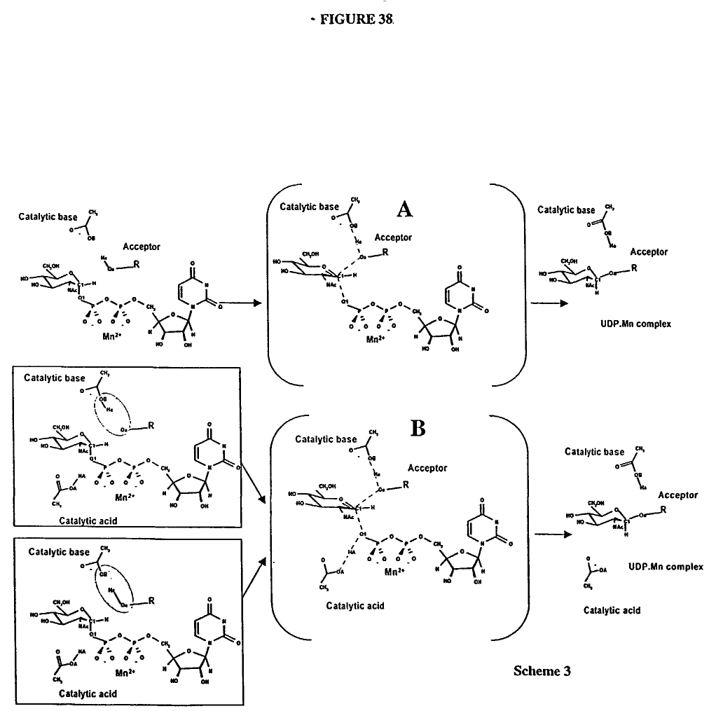

By analogy with the reaction mechanism of the inverting glycosyl hydrolases (18-20), one can assume two different types of mechanism for the inverting GlcNAc-Ts (FIG. 38). In the first type (Scheme A), only a carboxylate acting as a general base catalyst is involved in the catalytic mechanism. Such a catalytic base assists the nucleophilic attack of the acceptor oxygen Oa on the anomeric carbon C1 of the donor to form a new glycosidic linkage C1-Oa. A pair of carboxylic acids is involved in the second type of catalytic mechanism (FIG. 38, Scheme B). This mechanism consists of an electrophilic attack of a carboxylic acid on the target oxygen O1 of the donor that cleaves the bond, followed by the nucleophilic attack of the acceptor oxygen Oa on the anomeric carbon C1 of the donor. Here, one catalytic acid behaves as a general acid catalyst protonating the glycosidic oxygen atom O1 while the second carboxylate acts as a general base catalyst deprotonating the nucleophilic oxygen Oa of the acceptor. Both types of catalytic reaction described above may proceed via one or several transition states and, in terms of course of events, both mechanisms can proceed either in a concerted or stepwise manner. In the absence of experimental information on the reaction pathway, calculation of the potential energy surfaces (PESs) describing these mechanisms would provide valuable insight on the kinetic importance of a particular pathway and on the structure and energy of stationary points, intermediates and transition states observed on these PESs.

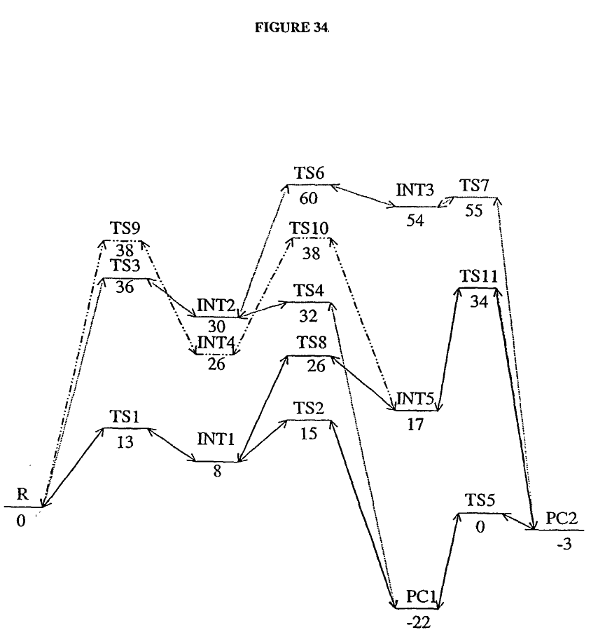

The HF/6-31G* calculated potential energy surfaces of the catalytic reactions are represented in the form of two-dimensional reaction-coordinate contour diagrams in FIGS. 1a-3a. The distances along the x-axis determine the formation and scission of a new glycosidic linkage C1-Oa, and corresponding to the nucleophilic attack of the acceptor Oa on anomeric C1, while the distances along the y-axis characterize the proton transfer processes. Different reaction pathways can generally be identified on these potential energy surfaces. The reaction pathways parallel to the vertical and horizontal axes describe particular steps in a stepwise mechanism while the reaction pathways following the diagonal across the PES represent a concerted mechanism. The profile of the PESs depends on the relative acidity of the different molecules involved. The calculated two-dimensional PESs only represent a section of the potential energy hypersurface (PEHS) describing the entire complex reaction. Nevertheless, several conclusions can be formulated from the calculated two-dimensional PESs displayed on FIGS. 1a-3a and they will be discussed below. Optimized structures of the different stationary points found along the reaction pathways, and determined at the DFT/B3LYP/6-31G* level, are given in FIGS. 1b-3b. Analysis of the stationary point structures along the reaction pathways calculated at DFT/B3LYP/6-31G* level revealed features qualitatively similar to those found on the PESs calculated at HF/6-31G* level. As previously observed (23-25), the inclusion of the electron correlation results in slightly different magnitudes of bond lengths and the increase of the basis set decreases the relative energy of minima. For that reason, the discussion will essentially be based on the structures calculated at DFT/B3LYP/6-31G* level and their energy estimated at DFT/B3LYP/6-31++G**//DFT/B3LYP/6-31G*. More detailed structural information on each stationary point is given for reference in Table 25 while their relative energy (ΔE) determined at various levels is listed in Table 26. FIG. 34 shows a scheme of various possible reaction pathways observed for the transfer of GlcNAc catalyzed by N-acetylglucosaminyltransferases.

3.1 Potential Energy Surfaces

PES as a function of the r_{Ha-OB} and r_{C1-Oa} distances. —The first reaction mechanism studied (FIG. 38, Scheme A) characterizes the nucleophilic attack of the methanol (sugar-acceptor) on the anomeric carbon C1 of UDP-GlcNAc (sugar-donor) either followed or preceded by the proton transfer from the methanol (acceptor) to the catalytic base (B). As mentioned earlier, only one carboxylate (catalytic base noted B on FIG. 37, Scheme 2) is involved in this reaction mechanism but for the sake of energy comparison with other reaction pathways, the second catalytic acid (noted A on FIG. 37, Scheme 2) was kept in the model in a constrained position. The PES corresponding to such reaction mechanism and calculated at HF/6-31G* level is given on FIG. 1a. Distances plotted along both axes of the contour map describe horizontally the nucleophilic attack of the methanol oxygen Oa on the anomeric carbon C1 of GlcNAc and vertically the proton (Ha) transfer from the hydroxyl group of the methanol to the catalytic base (B). This PES shows two intermediates (INT1 and INT2) and four energy barriers (TS1-TS4) located in valleys along the borders of the contour map. An energy maximum, with no saddle point, is observed in the central region of the map. Thus, a concerted mechanism appears to be impossible in this model and the reaction must proceed through a stepwise mechanism from the reactants (R) to the product complex (PC1) with the proton transfer and the nucleophilic attack as two distinct steps.

The stepwise mechanism observed on the contour map of FIG. 1a, offers two distinct pathways leading to the same product complex (PC1) but differing in the sequence of the individual steps. In the first pathway (R→TS1→INT1→TS2→PC 1), the enzymatic reaction starts with the nucleophilic attack (along the horizontal axis) of the methanol oxygen Oa on the anomeric carbon C1 of UDP-GlcNAc, followed by the proton (Ha) transfer (along the vertical axis) from the methanol to the catalytic base (B). In the second pathway (R→TS3→INT2→TS4→PC1), the order of the steps is reversed with the proton transfer occurring before the nucleophilic attack. Comparison of the energy barriers required to proceed along these two pathways (FIG. 34) reveals that the process starting by the nucleophilic attack is less energy demanding. From FIG. 1a and Table 26, both intermediates (INT1 and INT2) appear to have also higher relative energy compared to the reactants or the products. As a consequence, the transition states are located in asymmetric positions next to the intermediates.

The energy of the proton transfer from methanol to the catalytic base depends on the stage of the nucleophilic attack and reflects the different acidity of both participants. As the nucleophilic attack proceeds, acid strength of the attacking methanol changes from about 15 for methanol to an approximate value of −5 for the protonated glycosidic oxygen in INT1 (29). The changes in the calculated proton transfer energy are consistent with this variation of the pKₐ. When the methanol is in the starting position (r_{C1-Oa}=2.8 Å), not attacking the anomeric carbon of UDP-GlcNAc, the proton transfer energy is 36.3 kcal/mol, which is in reasonable agreement with the experimental estimate of ΔH=44 kcal/mol for the HCOO⁻+C₂H₅OH→HCOOH+C₂H₅O⁻process in the gas phase (29). The proton transfer energy then gradually decreases as the methanol oxygen Oa attacks the anomeric carbon C1 to finally end up with an energy around −22.4 kcal/mol in the final stage of the nucleophilic attack (r_{C1-Oa}=1.5 Å). The examination of the PES indicates that along the different pathways, the points located at equal r_{C1-Oa}=2.0 Å but with distinct position of the proton Ha, r_{Ha-OB}=1.0 Å and 2.0 Å respectively, appear to have roughly the same energy. This suggests that at such an r_{C1-Oa} distance, the acidity of the methanol and the catalytic base are very similar. The hydroxyl group of the methanol used in the model probably has higher acidity compared to the hydroxyl group that would be present in the real oligosaccharide substrate. It can, therefore, be expected that a weaker acid ROH of this type would increase the transition barrier and move TS1 and TS3 toward the products. Similarly, an increase of the strength of the catalytic base would move the TSs closer to INT2 or INT1.

Since the general reaction can be considered as a simple nucleophilic displacement at the anomeric carbon with inversion of configuration, the reaction path must involve the deprotonation of the acceptor oxygen and a change of the absolute configuration at the anomeric carbon. These changes are clearly seen in the structure of the discrete points along both reaction pathways (FIG. 1b). The analysis of these geometrical changes revealed that while the proton transfer only marginally influences the structure of the reactants, the nucleophilic attack results in a significant alteration of the UDP-GlcNAc structure. Along the reaction path: R→TS1 (13.4 kcal/mol)→INT1 (7.6)→TS2 (14.7)→PC1 (−22.4), the C1-O1 bond length between the anomeric carbon C1 and the leaving group, UDP, gradually elongates from 1.519 Å to 3.260 Å as the distance between the anomeric carbon and the attacking oxygen r_{C1-Oa} decreases. The transition state for the nucleophilic attack occurring as a first step in the reaction, TS1, undergoes significant geometrical changes compared to the starting structure, R. As the C1-Oa reaction coordinate gets close to 2.16 Å such as in TS1, the C1-O1 scissile bond increases drastically by 1 Å going from 1.519 Å to 2.535 Å and the C1-O5 bond shortens from 1.371 Å to 1.290 Å. In INT1, the C1-Oa and C1-O1 bonds have value of 1.532 Å and 2.829 Å, respectively. During the second step of the reaction, the proton Ha of TS2 is positioned closer to the oxygen Oa with r_{Ha-Oa}=1.361 Å. The C1-O1 distance slightly stretches to 3.014 Å whereas the C1-Oa bond shortens to 1.499 Å but in overall, only small changes were found between the relevant bonds of TS2 and INT1.

The geometry of the starting active site model (R) is characterized by the values of 1.519 Å and 1.371 Å for, respectively, the C1-O1 and C1-O5 bond lengths. The pyranoid ring of the GlcNAc is initially in the ⁴C₁ chair conformation characterized by the ring-puckering parameters φ=246.2, θ=14.1 and Q=0.54. Along the reaction coordinate, the conformation of the pyranoid ring continuously changes from the ⁴C₁ chair through the ⁴H₃ half-chair and the ⁴E envelope conformations where the proton H1 is at a quasi-planar position and back to the ⁴C₁ chair conformation. During this process, the HI atom moves from the equatorial position through the position in the plane defined by C2-C1-O5 atoms to the axial position. Interestingly, no boat conformations of the transferred sugar, as earlier described for β-1,4-xylanases (30) or suggested for chitinases (31), were found on the PES. These modifications in the six-membered ring conformation of GlcNAc are accompanied by changes in the orientation of the leaving and attacking groups with respect to the six-membered ring. As the ring shape shifts to the envelope conformation, the atoms attached to the anomeric carbon become coplanar with the sp² character at the reaction center, C1. The delocalization of the ring oxygen lone-pair electrons into the empty p orbital at the C1 atom stabilizes the oxocarbenium ion-like character of GlcNAc. The formation of such an oxocarbenium ion requires an alteration of the GlcNAc ring conformation, from chair to half-chair or envelope, to accommodate the partial double-bond character. A consequence of the charge delocalization is the shortening of the C1-O5 bond length from its equilibrium value of 1.371 Å observed in R. This change is more pronounced in TS1 with r_{C1-Oa=}2.158 Å and where the C1-O5 bond length, r_{C1-O5}=1.290 Å, developed a partial double bond character. The orientation of both the leaving and the attacking groups, is also influenced by the tendency to optimize interactions between the C1 carbon p orbital and the lone pairs of the connecting oxygen atoms of these groups. The most efficient interactions clearly occur when these oxygen atoms are located in the direction of this p orbital oriented perpendicularly to the O5-C1-C2 plane. A stronger nucleophilic character of the methanolate should result in a larger stabilization of such oxocarbenium species. Indeed, it appears that in the case of methanol, the O1 and Oa atoms adopt a quasi-orthogonal orientation regarding the O5-C1-C2 plane with the 5-C1-O1/Oa bond angles close to 90° at r_{C1-Oa}=2.158 Å, whereas in the case of methanolate (the alternative pathway described on the map), this situation occurs earlier at larger C1-Oa distance (r_{C1-Oa}=2.426 Å).

For the alternative reaction pathway described on the map, R→TS3 (36.3)→INT2 (30.4)→TS4 (32.2)→PC1 (−22.4), the conversion of the reactants into the intermediate INT2 during the proton transfer occurs through the transition state TS3 at r_{Ha-Oa}=1.638 Å. The geometry of GlcNAc does not exhibit any important change along this reaction step. The C1-O1 bond is a good illustration of this behavior since this bond remains almost unchanged with lengths of 1.519, 1.524 and 1.529 Å in respectively the R, TS3 and INT2 stationary points.

As in the first pathway, the main geometrical changes are connected with the nucleophilic attack occurring along the horizontal axis of the contour map. Several interesting differences between the TS1 and TS4 structures can be noticed. In TS4, the distance C1-Oa of 2.426 Å is longer compared to the 2.158Å found for TS1. On the contrary, the length of the C1-O1 scissile bond is considerably shorter in TS4, 1.863 A versus 2.535 Å in TS1. Using these distances as a criterion to describe the extent of the transfer reaction, one can assume TS4 is an earlier transition state because its geometry is closer to the reactants, in contrast to TS I that might be characterized as a late transition state since the structure is nearer to that of the products. Both TS1 and TS4 structures have a significant Sp² character at the C1 atom.

A comparison of the orientations of the N-acetyl group located at C2 shows that for all points on the PES, the acetamido group remains in the most stable conformation called Z-trans (32). This indicates that the N-acetyl group does not participate in the catalytic mechanism through the so-called substrate assisted catalysis by stabilizing the developing oxocarbenium character on C1 as proposed earlier for some retaining hydrolases (33). This is not surprising given the difference in stereochemical outcome. For some early points along the R→TS1→INT→TS2→PC1 reaction pathway, the N-acetyl group is brought closer to the leaving UDP group. However, the weak (N)—H . . . O1 hydrogen bond formed disappears as the C1-O1 distance increases.

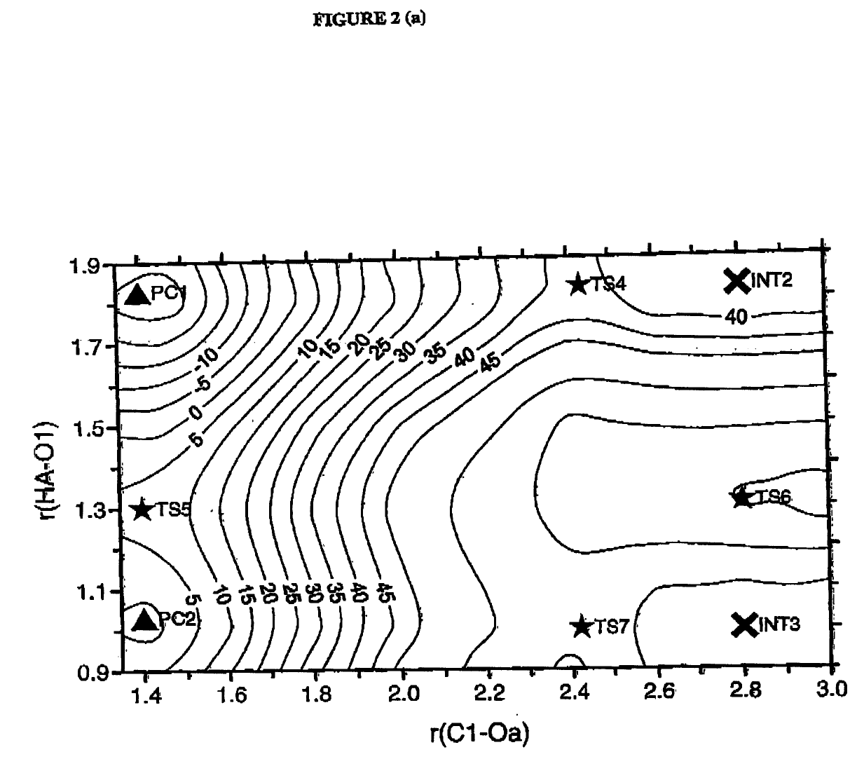

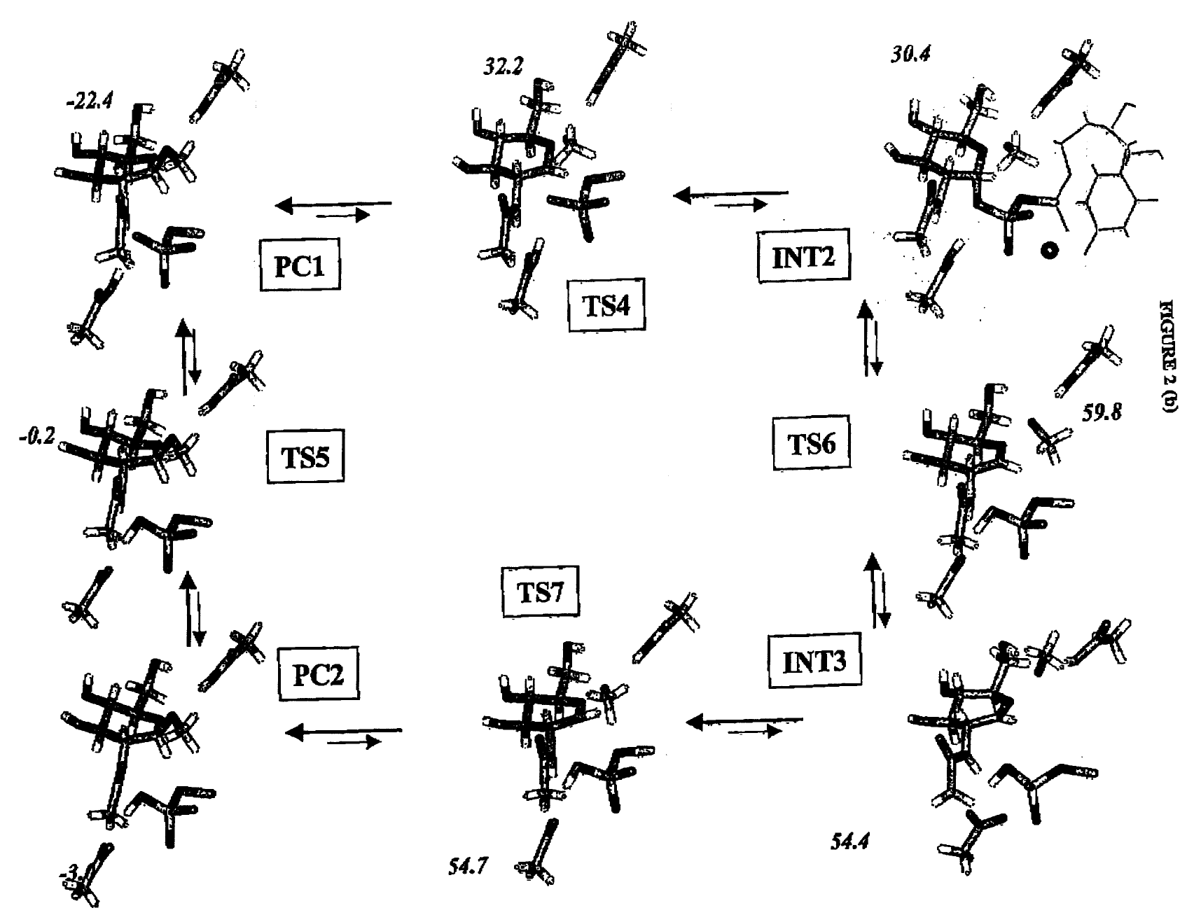

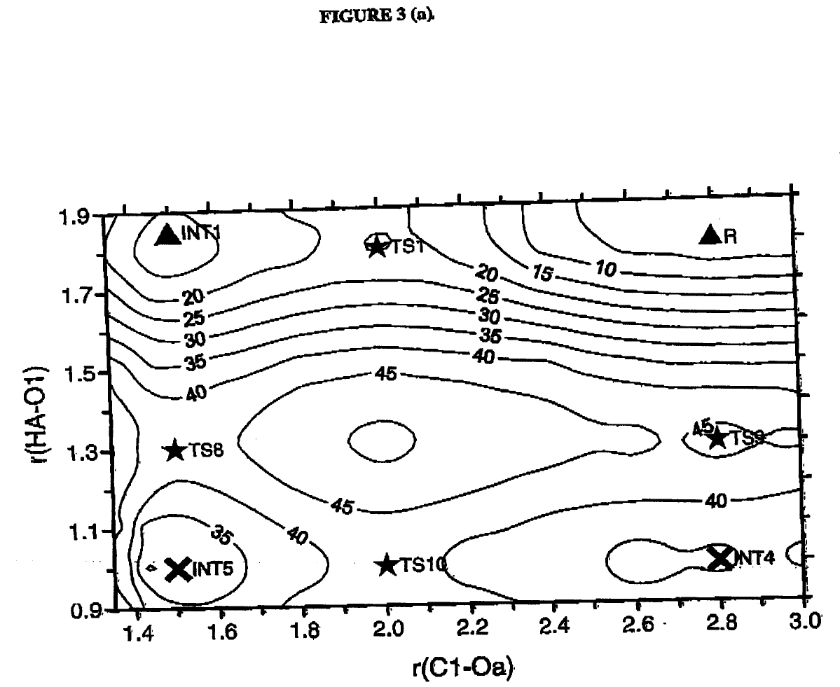

PESs as a function of the r_{HA-O1}, and r_{C1-Oa} distances. —The mechanism considered here describes the proton transfer from the catalytic acid (A) to the glycosidic oxygen O1 and the nucleophilic attack of the acceptor oxygen Oa on the anomeric carbon C1 (FIG. 38, Scheme B). Results on the mechanism earlier described showed that the nucleophilic attack and the proton transfer from the acceptor to the catalytic base (B) proceed in distinct steps. The question then was whether or not this behavior also remained for pathways where the HA proton of a second catalytic acid (A) is attacking the glycosidic oxygen. Preliminary calculations of the PES as a function of the r_{HA-O1} and r_{C1-Oa} distances indicated a conservation of these features. However, the optimization led to structures with the Ha proton located either at the acceptor oxygen atom (Oa) or at the catalytic base oxygen (OB) depending on the starting position. For that reason, two different PESs were calculated in order to describe the present mechanism. In the first PES (FIG. 2a), the Ha is initially located at the acceptor oxygen Oa while in the second PES (FIG. 3a), the proton Ha is positioned at the catalytic base oxygen OB. The analyses of the results support the previous findings and they reveal that on both potential energy surfaces, the Ha proton always remains in its same starting location. Some assumptions are implicitly included in these two models used to describe the reaction mechanisms. For the first PES (Ha located at the acceptor), the proton transfer from the acceptor to the catalytic base is the final step completing the reaction. However, in the second PES (Ha located at the catalytic base), the proton transfer from the acceptor to the base (B) precedes the nucleophilic attack and the proton transfer from catalytic acid (A) to the glycosidic oxygen O1.

Both PESs corresponding to the type of mechanism described on Scheme 3-B and calculated at the HF/6-31 G* level, are given on FIGS. 2a and 3a. The distances r_{C1-Oa} and r_{Ha-O1} represented along both axes of the contour map characterize horizontally the nucleophilic attack of the methanol oxygen Oa on the anomeric carbon C1 of GlcNAc and vertically, the proton (HA) transfer from the catalytic acid (A) to the glycosidic oxygen O1. Though the proton transfer processes represented on the vertical axes of FIG. 1 and FIGS. 2-3 refer to a transfer between different molecules, all maps exhibit similar features. They all indicate that a concerted mechanism is impossible in this model. As a consequence, the reaction must proceed by a stepwise mechanism though different reaction channels with the proton transfer and the nucleophilic attack occurring as two distinct steps. In all maps, the transition states are similarly located in asymmetric positions near the intermediates.

Two different pathways are observed on each of the PESs, namely INT2→TS4→PC1→TS5→PC2 and INT2→TS6→INT3→TS7→PC2 on FIG. 2 and R→TS1→INT1→TS8→INT5 and R→TS9 INT4→TS10→INT5 on FIG. 3. Three intermediates and four transition states are encountered in valleys along the borders of each contour map. It should be pointed out that some points of these PESs, corresponding to the nucleophilic attack with the HA proton located at the catalytic acid, coincide with points shown on FIG. 1. This was possible because the second catalytic acid (A) was kept in the model in a constrained position though it is not involved in the mechanism illustrated on FIG. 1. In this way, a consistent comparison between the energies required by the diverse reaction pathways was achieved.

The calculated PESs show that the HA proton must pass through a relatively large energetic barrier during its transfer from the catalytic acid (A) to the glycosidic oxygen O1. In general, this step is the most energy demanding among all the steps occurring along a particular reaction pathway. When this process is the starting step of a reaction mechanism, the energy barrier calculated at the 6-31 G* level is particularly high. For example, in the pathway R→TS9 (26.2 kcal/mol)→INT4 (25.8)→TS10 (37.7)→INT5 (17.3) (FIG. 3a), the barrier for proton transfer from the catalytic acid to the glycosidic oxygen (R→INT4) approaches 37.6 kcal/mol. A similar magnitude for the energy barrier between R→INT2 (36.3 kcal/mol) was observed in the mechanism earlier described and corresponding to the Ha proton transfer (FIG. 1). This suggests that in the present mechanism, the probability of protonation of the glycosidic oxygen might be too low to be kinetically productive. As on FIG. 1, the more favorable pathways found on FIGS. 2 and 3 are those starting with the nucleophilic attack.

The difference between the nucleophilic character of methanol and methanolate oxygen atoms is clearly reflected in the location of the transition barriers on PESs given in FIGS. 2a and 3a. When the nucleophile is methanolate (FIG. 2a), the reaction barriers for the nucleophilic attack are closer to the starting reactants (INT2), whereas, in the case of nucleophilic attack by methanol (FIG. 3a), the TSs are closer to the products. Barriers for the proton (HA) transfer from the catalytic acid (A) to the glycosidic oxygen O1 are located asymmetrically on the bottom part of the maps, closer to the final intermediates, and are therefore exhibiting a character of late transition states. The location of these energy barriers results from the fact that the glycosidic oxygen O1 in UDP-GlcNAc might have a pKa value (34) of approximately −10 and that the pyrophosphate group is a very strong acid. The activation energy of the reverse reaction in solution (the proton transfer from O1 to the catalytic acid) is an exothermic process assumed to be a diffused-controlled reaction with an activation barrier of about 5 kcal/mol (35), which is in reasonable agreement with the energy barriers of about 6 kcal/mol calculated on these maps, with however, an exception for the proton transfer between INT4→TS9.

As expected, the analysis of the geometrical changes observed along the reaction pathways and shown on FIGS. 2b and 3b reveals that the nucleophilic attack alters the structure in a similar fashion to the mechanism described on FIG. 1. However, the proton transfer from the catalytic acid to O1 is more significantly influencing the structure of UDP-GlcNAc than was in FIG. 1, the proton transfer from the acceptor to the catalytic base. The modifications in the structure of UDP-GlcNAc caused by the proton transfer can be illustrated by the bond distance r_{C1-O1} between the anomeric carbon C1 and the oxygen O1 that gradually elongates as the HA proton approaches the O1 atom. Such a process is observed on FIG. 3a during the R→INT4 step, where the value of the C1-O1 bond length changes from the equilibrium position of 1.519 Å through 1.950 Å in TS9 to 3.733 Å in INT 4. In the INT2→INT3 step described in FIG. 2a, the elongation of the glycosidic bond gets more pronounced with the C1-O1 distance increasing from 1.529 Å to 2.663 Å. The reverse trend is however observed for the C1-05 bond that is shortening from 1.371 Å to 1.259 Å along R→INT4 and from 1.381 Å to 1.253 Å along INT2→INT3. The changes in the bond lengths observed during this proton transfer process are accompanied by an alteration of the six-membered ring conformation. The conformation of the pyranoid ring continuously changes from the ⁴C₁ chair to the ⁴H₃ half-chair and back to the ⁴C₁ conformation. During this process, the HI atom moves from the equatorial position through the position in the plane defined by C2-C1-O5 atoms to reach ultimately the axial position. These results show that the structure around the reaction center is very sensitive to the nucleophilic attack and the proton transfer to the glycosidic oxygen. Moreover, they suggest that geometric changes caused by these two processes are comparable, both leading to the cleavage of the C1-O1 bond and the alteration of the GlcNAc ring conformation leading to inversion of the anomeric configuration of the transferred sugar. The energetic requirements for the two processes are very different and they indicate that the nucleophilic attack is the less energy-demanding operation.

Using information coming from the three calculated PESs, various possible reaction pathways for the transfer of GlcNAc catalyzed by N-acetylglucosaminyltransferases could be established. They are schematized in FIG. 34. Among them, four distinct stepwise reaction pathways exist to describe the transfer of GlcNAc using mechanisms involving jointly a catalytic acid and a catalytic base. Two pathways, R→TS3 (36.3)→IN12 (30.4) →TS4 (32.2)→PC1 (−22.4)→TS5 (−0.2)→PC2 (−3) and R→TS3 (36.3)→INT2 (30.4)→TS6 (59.8)→INT3 (54.4)→TS7 (54.7)→PC2 (−3), start with the proton transfer from the acceptor to the catalytic base, R→TS3→INT2, and as such, have their first step (noted in bold) identical to the mechanism previously discussed and involving only the catalytic base. In two other pathways, R→TS1 (13.4)→INT1 (7.6)→TS8 (26.4)→INT5 (17.3)→TS11 (34.4)→PC2 (−3) and R→TS9 (37.6)→INT4 (25.8)→TS10 (37.7)→INT5 (17.3)→TS11 (34.4)→PC2 (−3), the final step of the reaction, INT5→TS11→PC2, is the proton transfer from the acceptor to the catalytic base. In the first of these two pathways, the proton transfer to O1 occurs as the second step after the nucleophilic attack and it proceeds from INT1 to the intermediate INT5 via TS8. Both TS8 and INT5 structures have the GlcNAc ring in a ⁴H₃ conformation and the C1-O5 bond length around 1.33 Å. The C1-O1 bond length differs though for these structures with 2.843 Å versus 3.467 Å for TS8 and INT5, respectively. In the second pathway, the proton transfer happens as the first step, from R to INT4 via TS9, and it is energetically less favorable. In this case, the C1-O1 and C1-O5 bonds of TS9 are 1.950 Å and 1.286 Å, respectively. For all points on the PESs, the acetamido group remains in the most stable conformation called Z-trans (32).

Several changes described above resemble those assumed in reactions catalyzed by glycosyl hydrolases. The calculated potential energy surface for a general acid catalyzed reaction of lysozyme (35) has suggested for the transition state [RC1-O1(H+)−R], C1-O1 bond lengths in the range of 2.5-2.6 Å. It was assumed that the larger values calculated for the C1-O1 bond lengths of UDP-GlcNAc reflect the better leaving character of the UDP group compared to sugar aglycons. The comparison of the six-membered ring conformational rearrangements calculated in the present study with those observed in reactions catalyzed by glycosidases is of particular interest. Enforced by either the protonation of the glycosidic oxygen or the nucleophilic attack on the anomeric carbon, the changes observed in the GlcNAc ring conformation of UDP-GlcNAc resemble to some extent those calculated for 2-oxanol (36). This molecule was used as a hexopyranose model in the investigation of transition state structures during the glycoside hydrolysis mechanism. Some major features, however, distinguish glycosidases from glycosyltransferases. For example, it is clear from the analysis of all the points located on the PESs that the GlcNAc ring does not adopt any of the boat conformations sometimes described for glycosidase mechanism (30, 31). Most likely, the restraints associated with the nucleophilic attack of the acceptor and with the inversion of configuration at the C1 atom prevent any large ring conformational changes along the reaction pathways, which is in agreement with the assumption of the least motion effect (37). Complexes of glycosidases with a substrate or a product, in which a sugar ring is substantially deformed. have been experimentally observed (30, 31, 38). Ring distortion induced by these enzymes in ground states has been assumed to be crucial for their reaction mechanism. On the other side, circular dichroism studies in solution of N-acetylglucosaminyltransferase V and its complex with UDP-GlcNAc (39) suggested that the UDP part alone of UDP-GlcNAc is tightly bound to the enzyme while the GlcNAc residue is simply weakly interacting with the enzyme. These findings are supported by X-ray structural data available on complexes of glycosyltransferases with UDP-sugars (8, 11, 12) showing only the location of the UDP part in the binding pocket. The position of the transferred sugar moiety could never directly be determined from experiments, which suggests that in the ground state, the GlcNAc residue, initially observed in the ⁴C₁ conformation, is only loosely bound to the enzyme.

The inclusion of the electron correlation by means of the DFT/B3LYP method at the 6-31G* level usually reduces the relative energy of the stationary points determined on PESs compared to HF calculations (Table 26). The largest shifts are usually found for the structures along the proton transfer process, what indicates the importance of the use of electron correlation to describe systems with hydrogen bonds. The relative energy of the stationary points at the best theory DFT/B3LYP/6-31++G**//DFT/B3LYP/6-31G* is further decreased usually by about 3 kcal/mol and in few cases as much as 8 kcal/mol compared to DFT/B3LYP/6-31G*//DFT/B3LYP/6-31G*. The inclusion of electron correlation also affects the geometry of the molecules by increasing the bond lengths by approximately 0.3 Å (Table 5) as has earlier been observed for similar compounds (23-25). Reaction pathways.

The calculated PESs show altogether the presence of five intermediates (INT1-INT5) and 11 energy barriers indicating thus the existence of 11 transition states (TS1-TS11). Considering the structures of the stationary points described on FIGS. 1b-3b and Table 25 as well as the relative energetic data listed in Table 26 and schematically shown in FIG. 34, a number of results on the mechanism of inverting N-acetylglucosaminyltransferases become immediately apparent:

A maximum of energy is observed in the central region of all the calculated PESs indicating that a concerted mechanism is impossible in this model of GlcNAc transfer reaction. Therefore, in order to move from reactants (R) to the product complex, the reaction has to proceed through a stepwise mechanism. To avoid confusion, it is emphasized that this concerns only the r_{C1-Oa}, r_{Ha-OB}, and r_{HA-O1} reaction coordinates used to define the reaction mechanism. The C1-O1 bond has not been considered as a reaction coordinate since it is assumed here that changes in this bond length are a consequence of the nucleophilic attack or the proton transfer. The results clearly show that the C1-O1 distance varies in a continuous manner with the r_{C1-Oa} and r_{HA-O1} distances as a result of the nucleophilic attack at C1 or the proton transfer to O1. Whether the prolongation of the C1-O1 bond and the nucleophilic attack at C1 and proton transfer to O1 proceed in concerted manner remains to be explored.

There exist a number of transition states and intermediates connected by several pathways associated with proton transfer between the enzyme and the substrates and the nucleophilic attack of the acceptor. From the six possible pathways described here, only one [R→TS3 (36.3)→INT2 (30.4)→TS6 (59.8)→INT3 (54.4) (54.7)→PC2 (−3)] appears very unlikely. In this pathway, the catalytic reaction begins with two consecutive energetically unfavorable proton transfers.

The overall activation energy of 38 kcal/mol calculated for the preferred pathway of the mechanism requiring the participation of a pair of carboxylic acids [R→TS9 (37.6)→INT4 (25.8)→TS10 (37.7)→INT5 (17.3)→TS 11 (34.4)→PC2 (−3)] is relatively high. This mechanism involves the proton transfer from a catalytic acid to the glycosidic oxygen. However, the energy results shown on FIG. 34, apparently suggest that for some enzymes, the involvement of this catalytic acid in the reaction mechanism might not be essential for the enzymatic catalysis. These conclusions are supported by experimental findings reported for the inverting mechanism of human fucosyltransferase V, where only one carboxylate residue functions as a general base catalyst. It has been shown (40, 41) that a single proton is “in flight” at the rate determining transition state. The secondary deuterium kinetic isotope effect for this reaction is consistent with a large degree of S_{N}l character at the transition state and therefore a largely dissociative mechanism.

The GlcNAc transfer mechanism assuming the enrolment of only a catalytic base [R→TS1 (13.4) INT1 (7.6)→TS2 (14.7)→PC 1 (−22.4)] appears to be the less energy-demanding pathway represented on FIG. 34. The overall activation energy calculated at the DFT/B3LYP/6-31++G** //DFT/B3LYP/6-31G* level is approximately 15 kcal/mol. The alternative pathway with the proton transfer to the catalytic base occurring as first step [R→TS3 (36.3)→INT4 (25.8)→TS4 (32.2)→PC1 (−22.4)] requires considerably higher overall activation energy 36 kcal/mol).

The comparison of the calculated reaction barriers with experimental data would be very instructive but, unfortunately, values of the rate constant k_{cat} determined for the various GTs are not available in the literature. Only k_{cat} values for blood group A and B glycosyltransferases(42) and for FucT V (40) have been reported. These k_{cat} values are in the range between 50 s⁻¹ and 0.1 s⁻¹. Using the phenomenological definition associating k_{cat} with the activation free energy, ΔG^{act}=−RTln(hk_{cat}/k_{B}T), activation barriers between 15 kcal/mol and 19 kcal/mol have been determined. These estimates are in reasonable agreement with the overall activation energy of about 15 kcal/mol calculated for the GlcNAc transfer mechanism via the [R→TS1 (13.4)→INT1 (7.6)→TS2 (14.7)→PC1 (−22.4)] pathway. This further supports the assumption that inverting N-acetylglucosaminyltransferases would prefer a general base catalyzed mechanism represented by such a pathway. However, without any additional experiment and because this agreement could be only fortuitous, it would be premature to completely exclude the possibility for the reaction to proceed via another pathway. Especially when similar activation barriers were calculated for several reaction pathways indicating that subtle changes in the microenvironment of the active site could change the overall reaction barrier of any of the pathways.

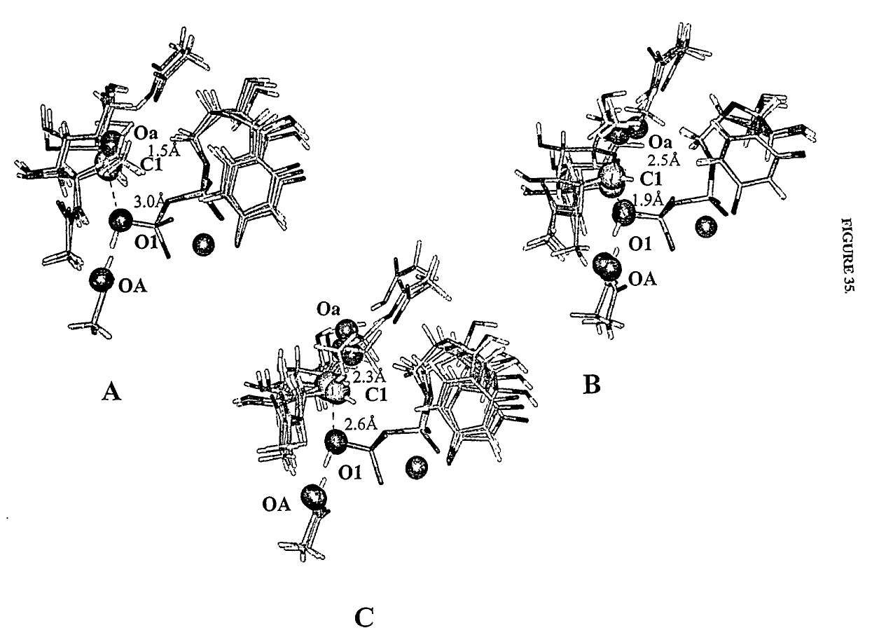

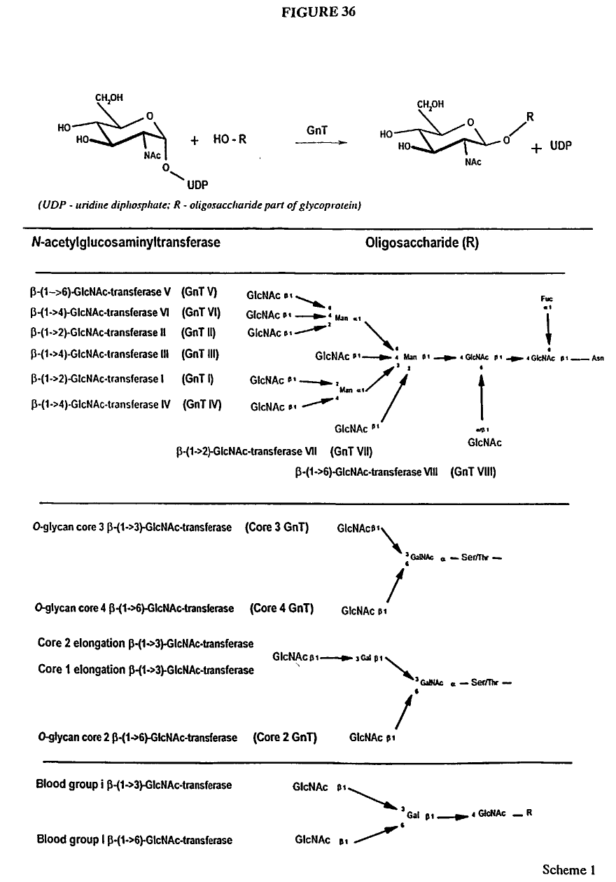

Transition state structures associated with the different reaction pathways exhibit significant variations in their C1-Oa and C1-O1 bond lengths (Table 25). Using these distances as criteria, the multiple transition states could be clustered into three groups presenting common structural features. The difference in C1-Oa and C1-O1 bonds of the TSs associated with different stages of the reaction and distinct pathways can be as large as 1.2 and 1.5 Å respectively. Superposition of the TSs belonging to each group is represented on FIG. 35. The first group (FIG. 35-A) is characterized by structures, such as TS2, TS5, TS8 and TS11, having short C1-Oa bonds within the range of 1.4-1.6 Å and long C1-O1 distances between 2.8 and 3.2 Å. The geometry of these stationary points is close to the structure of the final products, PC1 or PC2, where the C1-Oa bond created from the nucleophilic attack of the acceptor on C1 almost reached its final length of 1.42 Å and the UDP group is leaving the reaction site. These structures can therefore be described as “late transition states”. The second group (FIGS. 35-B) is represented by structures, such as TS3, TS4, TS9 and TS 10 with long C1-Oa bonds located within the range of 2.4-2.7 Å an C1-O1 distances comprised between 1.5 and 2.1 Å. The geometries of these stationary points have not been yet altered by the reaction and thus, are very comparable to the initial reactants. These structures can be termed as “early transition states”. The third group (FIGS. 35-C) corresponds to intermediate structures such as TS1, TS6 and TS7 where both C1-Oa and C1-O1 are elongated compared to their initial values but where the structures did not reach yet the final arrangement observed in the products, PC1 and PC2. Compared to the ⁴C₁ chair conformation in the reactants, the shape of the GlcNAc ring in most of the transition states changes to half-chair. These variations in C1-Oa and C1-O1 bond lengths and in the ring shape of the GlcNAc residue clearly demonstrate that the design of a transition state analog inhibitor is dependent on the actual mechanism of a particular enzyme.

Though the active site model consists of all the molecules that may directly be involved in the mechanism, it does only represent a model of the real active site and as such it has its internal limitations and several factors can influence the calculated relative energies. The actual arrangement of the relevant molecules as well as their conformation in the real active site might differ from the model considered but it can also vary from enzyme to enzyme. The real location of the catalytic acids in a particular enzyme may be different compared to the model. This may result, for example, in a smaller r_{Oa-OB} distance between the catalytic base and the hydroxyl group of the oxygen of the acceptor. As a consequence, the reaction barrier for the proton transfer from the acceptor to the catalytic base might be lower.(43) Reaction barriers for proton transfer and nucleophilic attack processes may also be drastically influenced by the presence in the vicinity (up to 6 Å) of the reaction center of ionized amino acid residues despite the fact that they might not participate in the reaction.(44) The influence of the catalytic metal on the conformation of the substrates is also a parameter to consider. An earlier study (25) showed that the relative stability of sugar-pyrophosphate conformations is sensitive to the occupancy of the metal coordination shell by interactions with surrounding elements present in the enzyme active site. This can be observed for instance, in the crystal structure of a nucleotide-complexed form of nucleotide-diphospho-sugar transferase SpsA, (11) where the coordination of the metal present in the active site, Mn²⁺, involves a neighboring aspartate residue.

Albeit ΔG values would be more appropriate for description of reaction processes, the conclusions were based on the energy values. For such a complex system like the reaction model, the determination of the ΔG for each individual step of the catalytic reaction would require enormous computational resources. On the basis of previous model compounds (23-25) that suggested inclusion of the ZPE and thermodynamic contributions to the calculated energies would only barely decrease energy differences, these calculations were not done. Therefore, it might be presumed that the calculated ΔE values could slightly be overestimated compared to ΔG values.

On the Catalytic Reaction Mechanism of Inverting N-acetylglucosaminyltransferases.

On the basis of the available experimental data and these calculation results, a mechanism of inverting N-acetylglucosaminyltransferases is suggested.

Experimental studies have revealed a sequential mechanism for GnT I and GnT II (6, 22) where UDP-GlcNAc binds first either to an enzyme.Mn²⁺ complex or as a Mn²⁺.UDP-GlcNAc complex followed by the binding of the acceptor substrate. Already observed in crystal (8) and in solution, (39) the binding of the sugar-donor appears to be associated with conformational changes of the enzyme upon binding of the sugar-acceptor. The binding of the nucleotide-sugar by the enzyme therefore triggers the conformational change that will bring to a proper distance the donor- and acceptor-binding sites in order to start the enzymatic reaction. Crystal structures of glycosyltransferases (8, 11, 12) and a solution study on GnT V (39) revealed however, that only the UDP part is observable in the nucleotide-sugar.enzyme complex. The sugar residue of the donor would then just be loosely bound in the ground state complex. Thus only the UDP-binding domain may be present or accessible in the sugar-donor.enzyme complex. This hypothesis can be supported by inhibition studies of GlcNAc-T II (6) indicating that neither GlcNAc nor GlcNAc-α-l-phosphate binds to the enzyme, whereas UDP-Glc, UDP-Gal, and UDP-GalNAc do bind into the enzyme though they are not substrates for GnT II. These results show how crucial is the UDP part for the binding of the donor into the enzyme, whereas it is mostly during the sugar transfer reaction that the enzyme recognizes the GlcNAc residue. UDP-GlcNAc being the GlcNAc-donor substrate of all GnTs, it is believed that the specificity of the enzyme must ensue from the structure of the transition state-binding domain that includes also specific information on the sugar-acceptor substrate.

In view of these different elements, one can therefore speculate that the sugar donor-binding site in N-acetylglucosaminyltransferases consists of two separate pockets: one pocket serving to accommodate the UDP part of the donor and a second for the binding of the sugar residue that will be transferred during the reaction. Only the UDP pocket would be occupied in the ground state. The sugar pocket of the donor-binding site would become accessible only after the reaction starts and the C1-O1 bond is elongated. Then only, in the transition state of the reaction, would the pocket be fully occupied. This pocket could more precisely be termed as the “sugar transition state pocket”. The UDP and sugar pockets should be separated by a distance corresponding approximately to the C1-O1 bond length in the transition state, which can be as large as 3.2 Å based on the calculations.

The architecture of the uridine-binding site, commonly known as a nucleotide recognition domain (NRD)(45), has been well described for many nucleotide-binding enzymes. A network of interactions involving the uracil and ribose rings with some conserved amino acids characterizes this region.(8, 11, 12) Therefore, it can be envisaged that the topology of the UDP-binding site may be fairly comparable in all concerned glycosyltransferases. An important feature in the UDP pocket is the presence of a metal cofactor, usually Mn²⁺, which is required by most of the UDP-dependent transferases for activity. The exceptions might appear to be β-1,6-GlcNAc-Ts, since their activity does not depend on the addition of a metal cofactor to the medium, however, it is assumed that these enzymes already contain a tightly bound metal ion.(1-4) The divalent cation contributes to the binding of UDP-GlcNAc in the binding site through strong interactions with the pyrophosphate group of the nucleotide. The nature of these interactions presumably determines the conformation adopted by the pyrophosphate group during the reaction. Without a doubt, the metal also plays an important role in the stabilization of the leaving group, UDP, which sees its formal charge changing from 0 to −1 as the C1-O1 bond is cleaved. It has been shown that a particular aspartate from the DXD motif, contained in many glycosyltransferases and involved in the binding of the nucleotide-sugar, is crucial for binding the divalent ion associated with the nucleotide. (46) A complex of UDP.Mn²⁺ was found in the products of galactosyltransferase reactions (28), leading to the notion that this particular Asp residue may also play an important role in the removal of the UDP-metal complex from the reaction site.

During the nucleophilic attack of the acceptor on the anomeric carbon, calculation of reaction pathways clearly showed the elongation of the C1-O1 linkage accompanied by conformational rearrangement of the glucopyranose ring. As the catalytic reaction proceeds, modifications at the reaction center move the sugar residue closer to the sugar-binding pocket. Interactions between the sugar and the enzyme benefit from this movement. As reactants get closer to the transition state, interactions with the enzyme increase and become crucial for the stabilization of the TS during the rate-limiting step. Relevant structural features within the enzyme active site should reflect the specificity and the structure of the transition state for a particular enzyme. The specificity for a particular sugar residue that differentiates UDP-GlcNAc, UDP-Glc, UDP-GalNAc, and UDP-Gal is likely produced by a sensitive array of hydrogen bonds, hydrophobic, and electrostatic interactions. Though the structure of the “sugar transition state pocket” is not experimentally known, one can hypothesize that this pocket will likely accommodate specific interactions with the proton-rich part of the half-chair sugar ring. Some amino acid residues located in the neighborhood of the N-acetyl group at C2 could also be responsible for the specificity of the enzyme, distinguishing for example between UDP-GlcNAc and UDP-Glc substrates. In the case of A/B glycosyltransferases, (42) the difference for the donor specificity, UDP-Gal vs. UDP-GalNAc, has been shown to reside in the different nature of a single amino acid, e.g. methionine vs. leucine, interacting favorably with the N-acetyl group of the donor. In the same way, properly oriented amino acids should preferentially interact with the equatorially oriented hydroxyl group at the C4 atom of GlcNAc and not with the axially oriented OH4 of GalNAc.

Concerning the acceptor-binding site, it should reflect the differences appearing in the oligosaccharide-acceptor structures, specific for each N-acetylglucosaminyltransferase, and what contributes to the specificity of the enzyme. In the case of a general base mechanism, a catalytic base, presumably an aspartate or a glutamate residue, is likely to be positioned at a proper distance from the hydroxyl group of the oligosaccharide-acceptor where the sugar transfer will occur. Inhibition studies of GlcNAc-T V revealed (47) that the α-D-mannopyranosyl residue to which GlcNAc-T V transfers is not tightly bound to the enzyme prior to the transfer of GlcNAc from the donor. This flexibility may allow the α-D-mannopyranosyl residue to adopt the optimal position for the nucleophilic attack.

Conclusion

Despite their extreme importance, the mechanism of glycosyltransferases has not yet been resolved but the results of this investigation have enlarged the understanding of this process. The present work explores the potential energy surface for the transfer of GlcNAc catalyzed by inverting N-acetylglucosaminyltransferases using ab initio quantum chemical methods. The structural model of the reaction site used in this study consists of all essential molecules assumed to be involved in the mechanism. All stationary points, transition states, and intermediates revealed from the calculated PESs were characterized at HF/6-31G*, HF/31++G**//HF/6-31G*, DFT/B3LYP/6-31G*, and DFT/B3LYP/6-31 ++G**// DFT/B3LYP/6-31G* levels. The multiple transition states along the different reaction pathways were grouped into three groups having common structural features relating them to different stages of the reaction. These geometrical differences clearly demonstrate that the design of a transition state analog inhibitor is dependent on the actual mechanism of a particular enzyme. Among the six different reaction pathways analyzed, a stepwise reaction pathway assuming the enrolment of only a catalytic base [R→TS1 (13.4)→INT1 (7.6)→TS2 (14.7)→PC1 (−22.4)] appeared to be the most probable reaction path, and is consistent with the existing experimental data. The mechanism described by such a pathway starts with the nucleophilic attack of the acceptor hydroxyl on the anomeric carbon C1 of the transferred GlcNAc, followed by proton transfer from the acceptor to the catalytic base.

The use of ab initio methods to study enzyme reactions presents a great challenge due to the high complexity and dimensionality of the potential energy surfaces describing this type of mechanism. Nevertheless, the recent progress in computational methods and technology makes possible to study more and more complex systems such as those presented in this work. The results obtained by these methods can supplement experimental data and provide unique information about reaction pathways and structure of the relevant stationary points observed along the reaction coordinate. Needless to say, such information is vital for the design of drugs inhibiting these enzymes.

### EXAMPLE 2

Structural Modeling of N-acetylglucosaminyltransferases

The structure of the enzyme is another relevant asset to design inhibitors in a rational way. In the absence of X-ray data, the modeling of the three-dimensional structure of some GnTs (Core2L and Core2b/M GnTs; GnT V; GnT Vb) provides an alternative to get insight on structural features of the active site. The modeling of these GnTs was carried out using computational procedures in the following systematic way:

(a) Search for homologous sequences in protein amino acid sequence databases and alignment of the amino acid sequences.

(b) Structural modeling of the protein using either the Threading or Homology approach.

(c) Refinement of the models using molecular mechanics.

(d) Docking of ligands into the active site of the enzyme.

(e) Analysis and description of the binding interactions for ligands.

(f) Search for ligand mimics in databases of protein-ligand complexes.

Information from the ab initio molecular orbital study described in Example 1 was also used in the modeling of the the glycosyltransferases.

Computational procedures.—Two different modeling procedures were used to generate models of GnTs. The first approach is so-called “protein threading”. This method has been used to successfully predict protein structures prior to their experimental studies. The appraisal of such approach is periodically reviewed at the “meetings on the Critical Assessment of Techniques for Protein Structure Prediction (CASP)”.(50) The threading approach is generally used when there is no structural or functional information on the sequence of interest. In this approach, a query amino acid sequence of a particular protein, in particular GnT, is mapped onto all the experimentally observed protein folds by using the publicly available Fold Recognition Server (51, 52). The fitness of such mapping is computationally based on a heuristic-scoring scheme that takes into account experimentally observed amino acid interactions. Based on their score, the best matching folds are selected, and the corresponding structural fragments are retrieved from the PDB database. (53) Subsequently, the protein sequence is mapped onto the different fragmentary structures. Though the generation of protein models using such procedure contains large risks, site-directed mutagenesis experiments and exhaustive docking calculations can validate the reliability of a particular model. The threading modeling approach has been used in the early stage of the investigation for generating models of GnTs. However, since the X-ray coordinates of SpsA glycosyltransferase (11) and GnT I (49) structures were obtained another approach, called “homology modeling”, has been used to predict the protein structures.

The homology procedure exploits the structural similarities between proteins by constructing a three-dimensional structure of a given sequence using as a template the structure of a similar and known protein. In this procedure, the amino acid sequence of the protein is matched onto the protein selected to be the template. The Needleman-Wunch alignment algorithm (54) was applied to align the two sequences of amino acids and the homology module from MS1 (55) was used to construct the enzyme models. First, the SpsA glycosyltransferase structure (11) was utilized as a template model for generating the homology models of GnTs. Subsequently the crystal structure of GnT I (49) became available, and it was used in the computational studies. In the created models, the atomic charges were assigned using the cvff91 force field and the essential hydrogen atoms fixed.

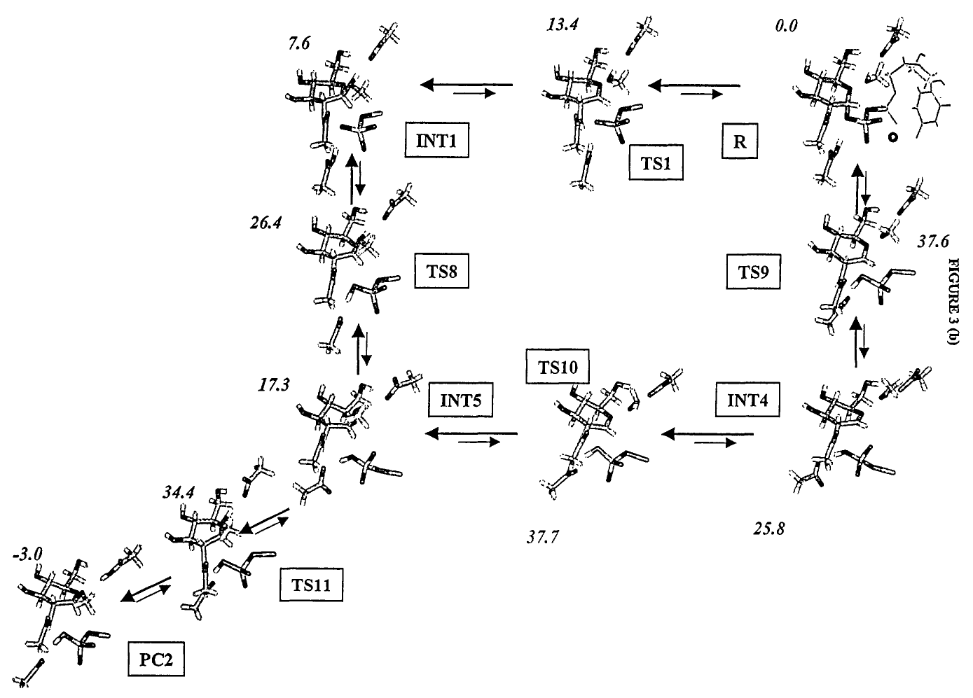

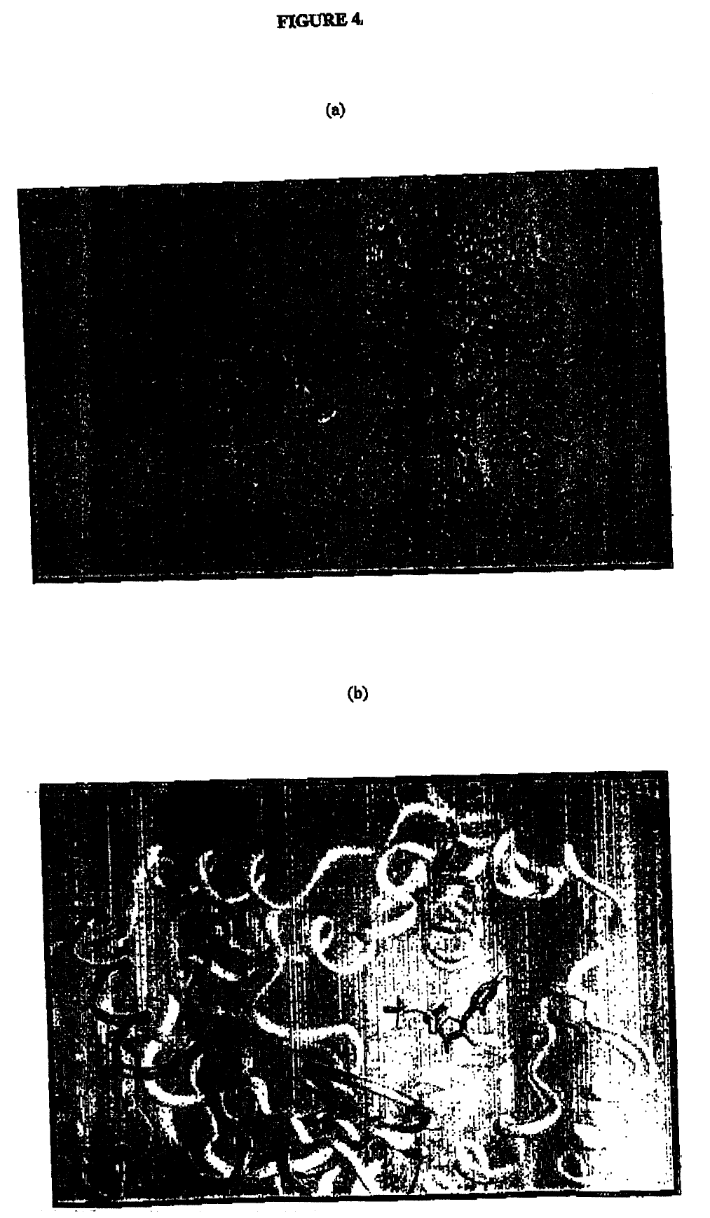

Topology of known GnTs.—The X-ray structures of GnT I and SpsA are shown on FIG. 4.a. The architecture of their binding sites appears somewhat similar. An exception is a loop consisting of amino acids 318-329 that is not clearly seen in the electron density map of GnT I (FIG. 4.b). In the SpsA structure, a loop similar to the sequence 318-329 of GnT I is folded into the active site and interacts with the nucleotide. The nucleotide-binding domain of SpsA is very similar to others, such as in pyruvate kinase and in 5′-3′ exonuclease domain of Thermus aquaticus DNA polymerase. The location of UDP was then determined using a docking procedure. Sequence alignments were performed for all GnT V, GnT Vb, Core2L and Core2b/M GnT enzymes. The best identity alignment, usually ˜30%, was used for further studies. To illustrate such procedure, the sequence alignment of Core2L GnT with GnT I is displayed on FIG. 5. On this Figure the relevant amino acid residues of the GnT I binding site are highlighted.

Homology modeling of Cote2L GnT, Core2b/M GnT and GnT V.—FIG. 6 shows the comparison of the homology models obtained for GnT V, Core2L and Core2b/M GnTs using as a template the GnT I structure. On this Figure, few relevant amino acid residues constituting the nucleotide-binding site accommodating the UDP part of the sugar-donor are shown in the form of tubes. The architecture of the active site was found to be similar for all three enzymes. Of course, some differences in the amino acid residues that form the active sites exist and they are discussed below.

### Modeling of N-acetylglucosaminyltransferase-Ligand Complexes

Insight on the crucial interactions between the enzyme and the natural substrates in the binding site is a prerequisite for understanding the catalytic mechanism of the enzyme and designing a specific inhibitor. To determine the interactions responsible for the binding of the ligands, the structure of GnT-ligand complexes was investigated using the docking approach.

Computational methods.—The protein-ligand docking calculations were performed using the AutoDock suite of programs (56). The position and orientation of the ligand into the protein was not constrained. All the freely rotatable bonds of the ligands were allowed to vary during the optimization runs of docking. About 100 docking runs were performed for every ligand. Each of these runs consists of about 1 million energy evaluations. Subsequently, the calculated complexes were clustered using a RMS tolerance of 2.5 Å and the top ranked clusters analyzed.

4.a. Critical Binding Sites for the Natural Substrates

The docking of the natural substrates into the active site of GnTs has been performed in a systematic manner. After docking UDP into the enzyme active site, the prominent binding modes for the nucleotide were identified and the lowest energy binding mode was used. Subsequently, the GlcNAc residue and the oligosaccharide-acceptor were docked into the protein active site. Three of the sub sites constituting the enzyme active sites, the UDP sub site, the GlcNAc sub site and the acceptor sub site, are briefly described in the following.

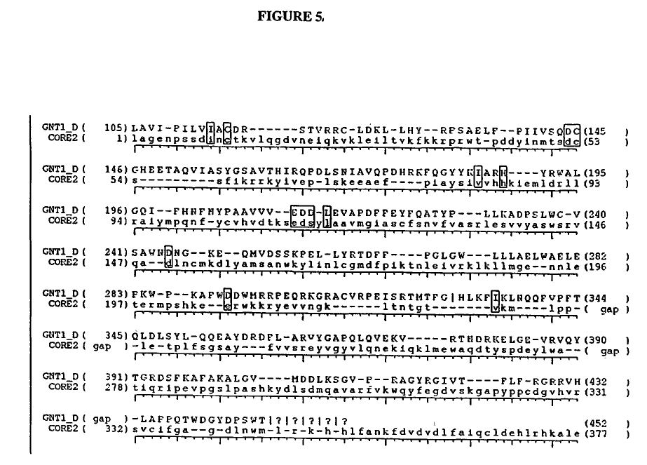

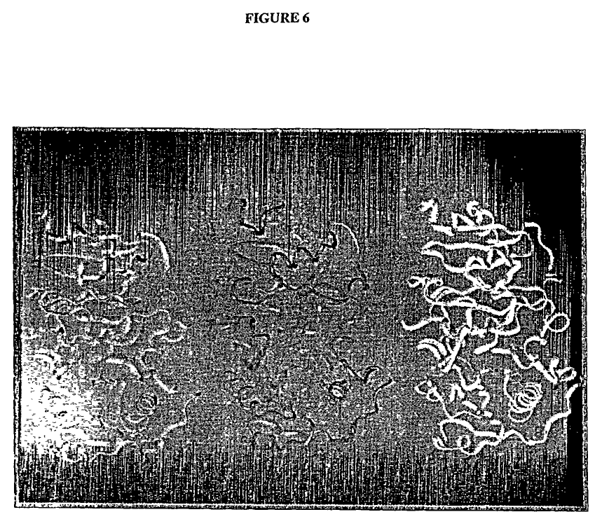

UDP-binding site.—Docking calculations of the UDP molecule onto the surface of the GnT I structure were undertaken. Guided by the SpsA structure were undertaken, the metal cation had earlier been placed in the active site of GnT I. Several possible binding modes for UDP were predicted. FIG. 7 shows the lowest energy binding modes of UDP within the electrostatic potential surface of GnT I. As it can be clearly seen from this Figure, UDP (shown in colored lines) binds into the well-formed electronegative groove where several interactions between the uridine and the side chains of GnT I stabilize the UDP location. A list of the predicted interactions is given in Table 10, 11, 12, and 13. This funnel shaped groove restricts the conformation of the uracil ring in a narrow pocket. While in most of the lowest energy binding modes calculated for UDP, the uracil group appears to prefer a similar and characteristic binding mode, at least three different conformations are observed for the pyrophosphate moiety. A comparison of the atomic details described therein on the GnT I-UDP complex with the model of the complex obtained by docking revealed that the predicted intermolecular interactions described in Table 10 are in agreement with the interactions experimentally observed. Similarly to the procedure above described for GnT I, the different homology models of GnTs were used to determine and characterize the binding site of the nucleotide sugar. A similar electronegative groove with the uracil part of UDP bound deeply into the pocket is commonly seen in the known X-ray structures of glycosyltransferases. Comparison of the observed and predicted interactions revealed that a characteristic pattern of interactions is conserved in all GnTs and suggests that this region is relevant for the recognition and binding of UDP-GlcNAc. This is illustrated on FIG. 8 where the interactions between the enzyme and UDP are displayed.

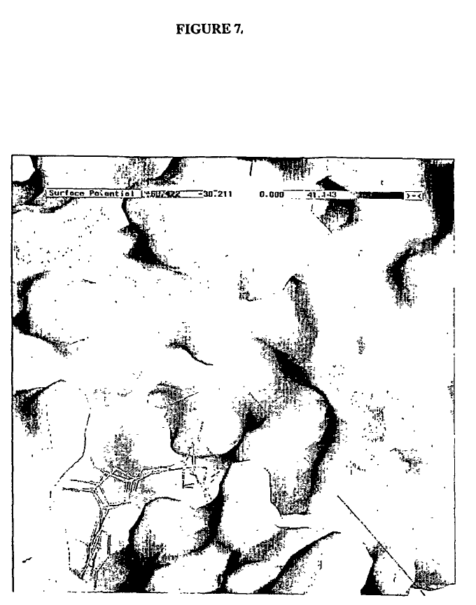

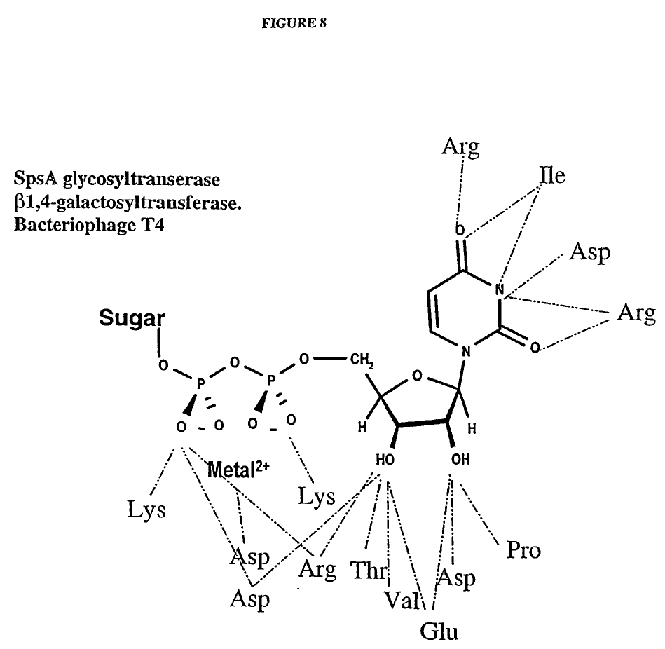

Based on the docking results, the UDP-binding site in Core2L GnT can be described in detail. A representation of the four top scoring Core2L GnT complexes with UDP is shown on FIG. 9. In the conformations displayed on this Figure, the uridine part of UDP favors a similar binding mode comparable to the one observed in GnT I, but the diphosphate moiety adopts two different conformations. In both of these phosphate conformations, the phosphoryl oxygens located the closest to the uridine are in the vicinity of a glutamate (Glu-159 in Core2L GnT) that is conserved in the pyrophosphate sub site through all the different GnTs investigated (Table 10, 11, 12, and 13) Residues Ile-57 and His-131 located in the surroundings of the ribose-binding pocket arrest the ribose in a definite location. These interactions, together with stacking interactions of the Cys59-Cys100 disulfide bond with the uracil, assist in restricting the uridine group in a favorable position to the protein. The conformational preferences observed for the pyrophosphate group indicate a possible flexibility of the pyrophosphate moiety. This flexibility may play an important role in the removal of the products from the active site.

Results from the docking of UDP into the homology model of GnT V gave a set of conformations that overlapped with the predicted UDP-Core2L GnT complex (Table 11, 12). However, the stacking interactions of the uracil group with the Cys59-Cys 100 disulfide bond were not observed in GnT 1. This seems to be the most relevant difference between the UDP-binding sites of GnT V and Core2L GnT.

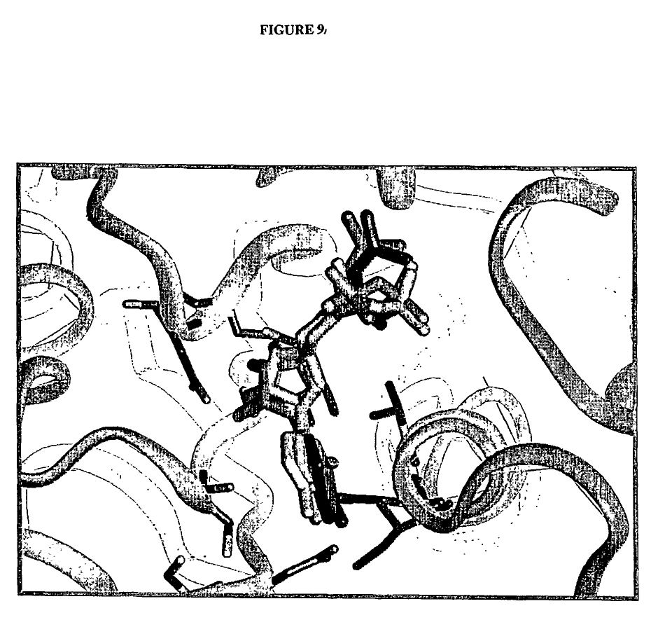

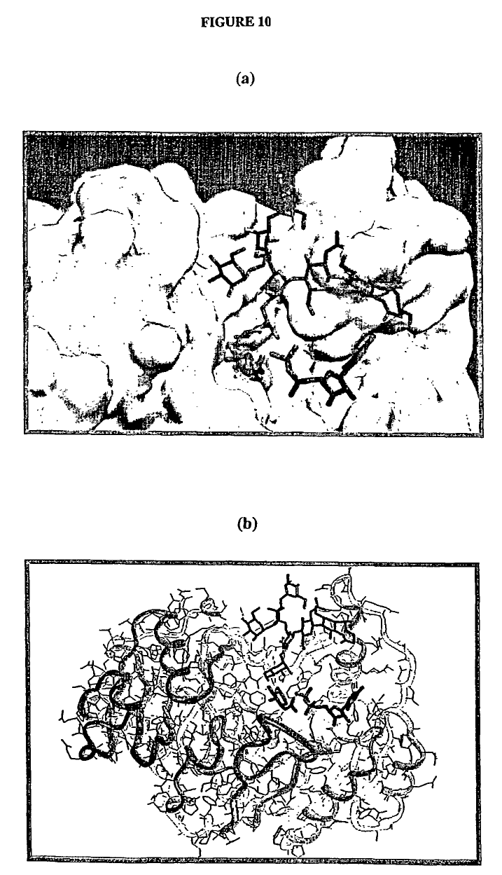

Transition state-binding site.—As an isolated molecule, GlcNAc appears to dock in many regions of the GnT I surface. The surface representation of GnT I is shown on FIG. 10. In GnT 1, the most significant binding site of the GlcNAc molecule corresponds to a specific hydrophobic pocket. The predicted lowest energy docking modes calculated for the acceptor heptasaccharide into the enzyme are shown on FIG. 10. The terminal mannose, to which the GlcNAc is transferred, is buried into the binding site, in the vicinity of the GlcNAc-binding site. Several docking modes for the acceptor are observed. The location of the amino acid Asp-291 in the vicinity of GlcNAc and the acceptor oligosaccharide suggests a possible function of catalytic base for this amino acid. Some of the low energy docking modes resemble the lowest energy complex displayed on FIG. 10, however, different arrangements of the other sugar units are observed, that creates distinct local interactions.

As for GnT I, the GlcNAc molecule in the isolated form, appears to dock in many regions of the Core2L GnT surface. FIG. 11 shows one of the predicted binding modes for the UDP-GlcNAc-Acceptor (GalNAc-Gal) complex. The most significant binding site of the GlcNAc molecule is a hydrophobic rich region located about 3 A apart from UDP. The estimation of this distance is done using the (GlcNAc)C1 . . . OP(UDP) distance. For Core2L GnT, the Glu-253 residue is assumed to play the role of the catalytic base in the reaction mechanism. On FIG. 11 the protein surface is color coded by the chemical nature of the atom. It is clear from this Figure that the entire ligand molecules dock into the cavity formed at the Core2L GnT surface. It is also noteworthy that the arrangement of the reactants in this binding mode resembles the structure of the late transition state predicted by the ab initio calculations.

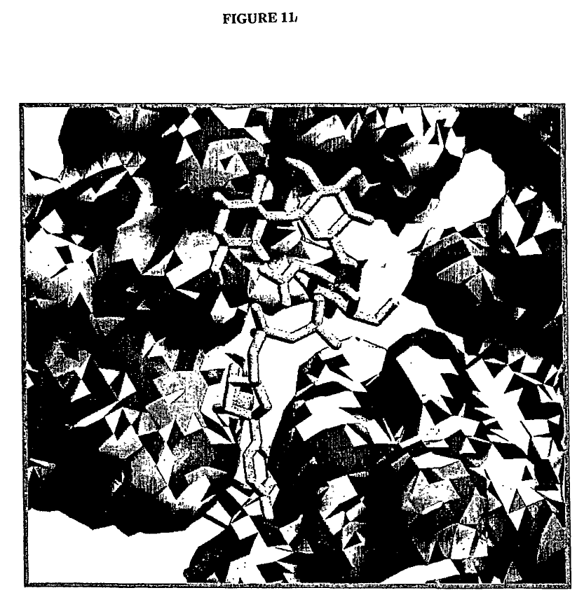

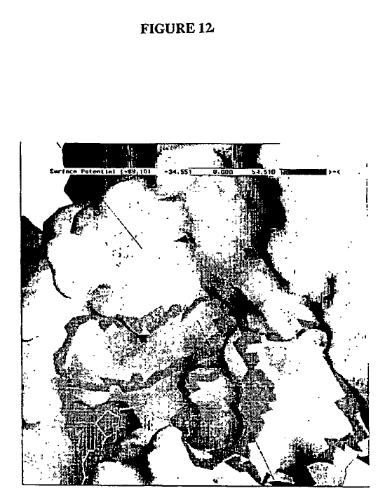

To summarize, the active site of the modeled Core2L GnT appears to have at least three prominent sub sites where the UDP, the GlcNAc and the oligosaccharide-acceptor can easily be accommodated. The GlcNAc and the oligosaccharide-acceptor regions are outlined in green on the electrostatic potential surface of the modeled Core2L GnT presented in FIG. 12. In this Figure, the UDP is shown in its preferred binding mode. The sub site located on the top of the ribose ring is the hydrophobic region where the GlcNAc binds. The other sub site corresponds to the region where the disaccharide-acceptor (GalNac-Gal) binds.

The results revealed that the UDP-binding site of the modeled Core2b/M GnT has similar features to those earlier described for Core2L GnT. The similar architecture of the active site in all GnTs studied is documented in Tables 10, 11, 12, and 13, where some relevant amino acid residues forming three sub sites of the active site are listed together with the predicted catalytic base residue.

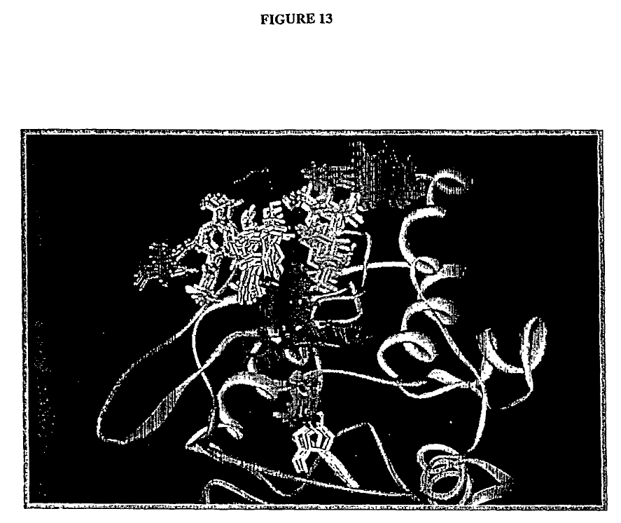

At the moment, results on the GnT V-transition state complexes are based on the model of GnT V issued from the SpsA structure. These results are given on FIG. 13 and they resemble the results on other GnTs revealing three distinct sub sites in the enzyme active site.

4.b. Binding of Selected Compounds into the Active Site of N-acetylglucosaminyltransferases

As a first step in understanding the potency and specificity of the GD compounds, several compounds were docked into the Core2L GnT model. Preliminary results indicate that GD compounds with a UDP-like group prefer to dock into a well defined pocket through two strong hydrogen bonds with Asp99 and His-131 and a strong stacking interaction with the hydrophobic amino acid residue, Ile-57, located at the bottom of the pocket. To illustrate the potential of the method used, the results obtained for GD0500, a fragment of GD0541 and a potential inhibitor are described.

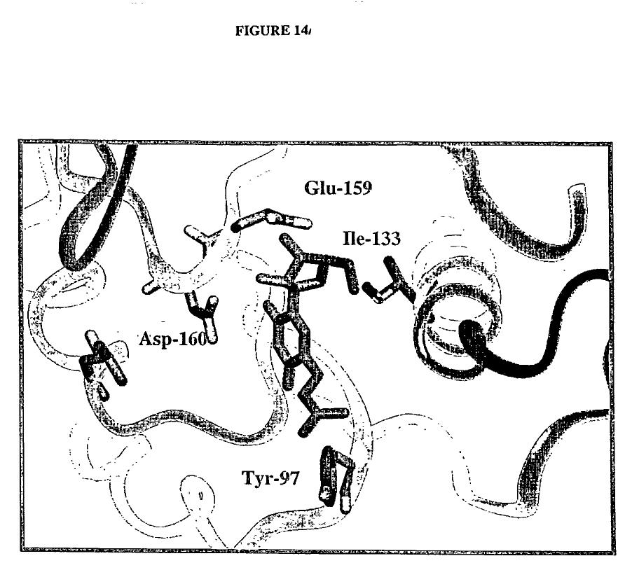

Binding mode of GD0500.—GD0500 is an analogue of the uridine molecule bearing a small modification at the C5 position of the uracil ring. The docking of this simple molecule into the active site of Core2L GnT led to four prominent binding regions. A location of the top ranking binding mode resulting from the docking calculations corresponds to the earlier described UDP-binding pocket of Core2L GnT (FIG. 14). This binding mode involves all the interactions observed for the binding of the uridine part of UDP. However, the orientation of GD0500 in the binding site is slightly different than for UDP. The substituent at the C5 position of the uracil ring of GD0500 favors a hydrogen bond interaction with Tyr-97 from Core2L GnT. This interaction might be responsible for the small shift in the orientation of GD0500 compared to UDP. In addition, this hydrogen bond interaction may perhaps be the reason for the specific inhibition of Core2L GnT. In fact, preliminary results show that Tyr-97 is not present in the models of GnT I and GnT V. The absence of this interaction in the enzyme-GD0500 complexes may explain the different inhibitory results of this compound against the different enzymes.

Binding mode of a GD0541 fragment.—GD0541 was found to be a potent inhibitor of Core2L GnT. Docking of a fragment of this molecule in the Core2L GnT model revealed various possible binding modes in several regions of the protein surface (represented in yellow color on FIG. 15). A detailed analysis of the docking calculations revealed the presence of a conformational cluster of the GD0541 fragment overlapped with the predicted binding region of UDP. In these computed GD0541 fragment-Core2L GnT complexes, the hydroxyl groups attached to the benzyl group of GD0541 fragment are situated in close distance to the metal ion. Independently, a structural database search revealed that similar hydroxyl groups attached to a benzyl ring generally favor interactions with a bound metal ion.

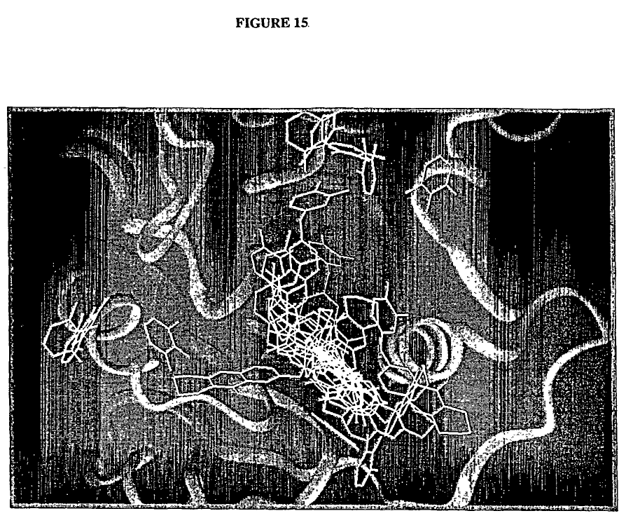

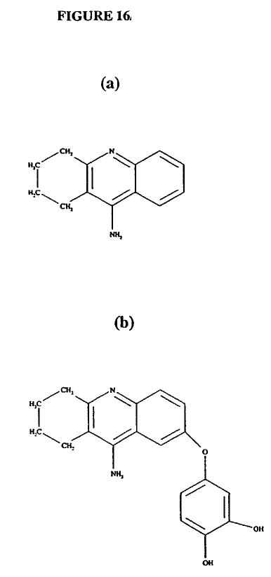

Binding mode of a potential GD0541 analogue.—Extensive database searches revealed that tetrahydroaminoacridine (Tacrine, FIG. 16.a) and its structural analogues prefer to dock into hydrophobic rich region. Interestingly, Tacrine is a drug already used for the treatment of Alzheimer's disease as an inhibitor of acetylcholinesterase. A close resemblance was detected between the GlcNAc-binding site in Core2L GnT and the Tacrine-binding sites in various protein complexes. This suggests that Tacrine could presumably occupy the hydrophobic region of GlcNAc. As a result of these observations, a molecule was built on the assumption that a combination of the GD0541 structural features with the hydrophobic moiety contained in Tacrine might lead to a significant restriction of the binding modes observed for the GD0541 fragment. Computational modification of GD0541 resulted in a new potential molecule having the Tacrine molecule attached to the fragment of GD0541 through an etheric linkage (FIG. 16.b). The docking results show that unlike the GD0541 fragment alone, this combination of fragments, displayed on FIG. 15 in white color, favors indeed the docking in a single region of the Core2L GnT model. This region appears also to be the prominent site where UDP binds. In the top scoring complexes, the Tacrine fragment binds into the GlcNAc-binding site while the hydroxyls of the GD0541 fragment interact with Glu-159 and Asp-160 via a metal ion. This suggests that this type of molecule could be tested as a potential lead for developing new inhibitors of Core2L GnT.

### Discussion and Conclusions

The present results shed some light on the mechanism of inverting N-acetylglucosaminyltransferases, the structure of the transition states, the structure and character of the catalytic site of GnTs, and the binding of natural substrates and inhibitors into the GnTs. As a result, the following conclusions can be drawn:

Among the six different reaction pathways analyzed, a stepwise reaction pathway assuming the enrolment of only a catalytic base appears to be the most probable reaction path that is consistent with the existing experimental data. A mechanism described by such pathway starts with the nucleophilic attack of the acceptor reactive hydroxyl on the anomeric carbon C1 of the transferred GlcNAc, followed by the proton transfer from the acceptor to the catalytic base. The structure of the rate determining transition state for this pathway corresponds to the “late transition state”.

The multiple transition states found along the different reaction pathways were classified into three groups having common structural features relating them to different stages of the reaction. During the reaction, the structure of the donor, UDP-GlcNAc, undergoes significant structural changes. The geometry of the so-called “late transition states” is close to the final products, where the C1-Oa bond (1.5 Å) created during the reaction almost reached the length of the C-O glycosidic bond (1.42 Å). The UDP group that is leaving the reaction site is characterized by the C1-O1 distance ˜3 Å. The structural characterization of the late transition state offers us an opportunity to create a pharmacophore and to design late transition state analogue inhibitors.

The binding interactions of the natural substrates (UDP-GlcNAc and acceptor-oligosaccharide) within the homology models of Core2L GnT, GnT V and GnT I were described. Based on the complex models, it is proposed that the binding sites of these enzymes possess at least three prominent binding sites. The models allowed the prediction of the position of an amino acid residue that functions as the catalytic base in the catalytic reaction mechanism. The characterization of the structure and interactions in each of the three binding sub sites can be used in a sub site-directed pharmacophore search.

In all the GnTs, the binding conformation of the uracil group in UDP is restricted because of the size of the funnel shaped groove and the nature of the amino acid residues forming the narrow groove. In all the predicted binding modes of UDP, the carbonyl oxygen atoms (02, 04) and the amide hydrogen (N3H) of the uracil ring interact with the protein while the CS position of the ring is exposed to the exterior.

The ribose ring prefers a very defined conformation in the active site of all GnTs. A hydrophobic residue at the bottom of the UDP-binding site pocket arrests the conformation of the ribose by favorable stacking interactions.

A “hydrophobic pocket” corresponding to the GlcNAc transition state-binding site has been identified. In all GnTs, the GlcNAc prefers to dock into this well-defined hydrophobic pocket, which is approximately 3 Å distant from the pyrophosphate-binding sub site.

Models of Core2L GnT complexed with some drug hits provide some structural explanation for their inhibitory activity.

Proposed catalytic mechanism of glycosyltransferases. —On the basis of the available experimental data, protein-ligand modeling, reaction pathways and transition state structure calculations, the mechanism of inverting N-acetylglucosaminyltransferases.

Experimental studies have revealed a sequential mechanism for GnT I and GnT II (6, 22) where UDP-GlcNAc binds first either to an enzyme.Mn²+ complex or as a Mn²+.UDP-GlcNAc complex followed by the binding of the acceptor substrate. Already observed in crystal (8) and in solution, (39) the binding of the sugar-donor appears to be associated with conformational changes of the enzyme upon binding of the sugar-acceptor. The binding of the nucleotide-sugar by the enzyme therefore triggers the conformational change that will bring to a proper distance the donor- and the acceptor-binding sites in order to start the enzymatic reaction. Crystal structures of glycosyltransferases (8, 11, 12) and a solution study on GnT V (39) revealed however, that only the UDP part is observable in the nucleotide-sugar.enzyme complex. The sugar residue of the donor would then just be loosely bound in the ground state complex. Therefore only the UDP binding domain may be present or accessible in the sugar-donor.enzyme complex. This hypothesis can be supported by inhibition studies of GlcNAc-T II (6) indicating that neither GlcNAc nor GlcNAc-α-1-phosphate binds to the enzyme, whereas UDP-Glc, UDP-Gal, and UDP-GalNAc do bind into the enzyme though they are not substrates for GnT II. These results show how crucial is the UDP part for the binding of the donor into the enzyme, whereas it is mostly during the sugar transfer reaction that the enzyme recognizes the GlcNAc residue. Since UDP-GlcNAc is the GlcNAc-donor substrate of all GnTs, the specificity of the enzyme must ensue from the structure of the transition state binding domain that includes also specific information on the sugar-acceptor substrate.

In view of these different elements and results of the molecular modeling, one can therefore consider that the sugar-donor binding site in N-acetylglucosaminyltransferases consists of two separate pockets. One pocket serving to accommodate the UDP part of the donor and a second for the binding of the sugar residue that will be transferred during the reaction. Only the UDP pocket would be occupied in the ground state. The sugar-binding pocket of the donor would only become accessible after the reaction starts and the C1-O1 bond is elongated. Only in the transition state of the reaction, would the pocket be fully occupied. This pocket could more precisely be termed as the “sugar transition state pocket”. The GnT homology structures complexed with ligands show that the GlcNAc pocket is very hydrophobic and separated by about 3 Å from the UDP pocket. This separation corresponds approximately to the average C1-O1 bond length of 3.1 Å observed in the late transition states that was independently calculated using ab initio methods.

The architecture of the uridine-binding site, commonly known as a nucleotide recognition domain (NRD)(45), has been well described for many nucleotide-binding enzymes. A network of interactions involving the uracil and ribose rings with some conserved amino acids characterizes this region. (8, 11, 12). The docking calculations with different GnTs show similar nucleotide-binding interactions where the uracil and ribose moieties are tightly bound to the enzyme. Therefore, it can be envisaged that the topology of the UDP-binding site may be quite comparable in the concerned glycosyltransferases. An important feature in the UDP pocket is the presence of a metal cofactor, usually Mn²⁺, which is required by most of the UDP-dependent transferases for activity. The exceptions might appear to be β-1,6-GlcNAc-Ts, since their activity does not depend on the addition of a metal cofactor to the medium, however, it is assumed that these enzymes already contain a tightly bound metal ion. (14). The divalent cation contributes to the binding of UDP-GlcNAc in the binding site through strong interactions with the pyrophosphate group of the nucleotide. The nature of these interactions presumably determines the conformation adopted by the pyrophosphate group during the reaction. Without a doubt, the metal also plays an important role in the stabilization of the leaving group, UDP, which sees its formal charge changing from 0 to −1 as the C1-O1 bond is cleaved. It has been shown that a particular aspartate from the DXD motif, contained in many glycosyltransferases and involved in the binding of the nucleotide-sugar, is crucial for binding the divalent ion associated with the nucleotide. (46) In agreement with these data, the GnT homology models (Tables 10 and 11) have usually a glutamate residue in the vicinity of the pyrophosphate-binding site. A complex of UDP.Mn²⁺ was found in the products of galactosyltransferase reactions, (28) which indicates that this particular aspartate residue may as well play an important role in the removal of the UDP-metal complex from the reaction site.

During the nucleophilic attack of the acceptor on the anomeric carbon, calculation of reaction pathways clearly showed the elongation of the C1-O1 linkage accompanied by conformational rearrangement of the glucopyranose ring. As the catalytic reaction proceeds, modifications at the reaction center move the sugar residue closer to the sugar-binding pocket. Interactions between the sugar and the enzyme benefit from this movement. As reactants get closer to the transition state, interactions with the enzyme increase and become crucial for the stabilization of the TS during the rate-limiting step. Relevant structural features within the enzyme active site should reflect the specificity and the structure of the transition state for a particular enzyme. The specificity for a particular sugar residue that differentiates UDP-GlcNAc, UDP-Glc, UDP-GalNAc, and UDP-Gal is likely produced by a sensitive array of hydrogen bonds, hydrophobic, and electrostatic interactions. Though the structure of the “sugar transition state pocket” is not experimentally known, one can hypothesize that this pocket will likely accommodate specific interactions with the proton-rich part of the half-chair sugar ring. Some amino acid residues located in the neighborhood of the N-acetyl group at C2 could also be responsible for the specificity of the enzyme distinguishing for example between UDP-GlcNAc and UDP-Glc substrates. In the case of A/B glycosyltransferases, (42) the difference for the donor specificity, UDP-Gal vs. UDP-GalNAc, has been shown to reside in the different nature of a single amino acid, e.g. methionine vs. leucine, interacting favorably with the N-acetyl group of the donor. In the same way, properly oriented amino acids should preferentially interact with the equatorially oriented hydroxyl group at the C4 atom of GlcNAc and not with the axially oriented OH4 of GalNac. Indeed, the docking modeling of UDP-GlcNAc into Core2L GnT supports these assumptions, showing a specific hydrogen bond between the OH4 from GlcNAc and Ser-l 58 and a stacking interaction of the N-Acetyl group from GlcNAc with Leu-163. Such interactions would not be possible in a different type of sugar residue (i.e. Glc, Gal, GalNAc or Man).

Concerning the acceptor-binding site, it should reflect the differences appearing in the oligosaccharide-acceptor structures, specific for each N-acetylglucosaminyltransferase, and what contributes to the specificity of the enzyme. In the case of a general base mechanism, a catalytic base, presumably an aspartate or a glutamate residue, is likely to be positioned at a proper distance from the hydroxyl group of the oligosaccharide-acceptor where the sugar transfer will occur. In the homology models, a catalytic base (see Tables 10, 11, 12, and 13) is usually present in the neighborhood of the GlcNAc and oligosaccharide acceptor moieties. Inhibition studies of GlcNAc-T V revealed (47) that the α-D-mannopyranosyl residue to which GlcNAc-T V transfers is not tightly bound to the enzyme prior to the transfer of GlcNAc from the donor. This flexibility may allow the α-D-mannopyranosyl residue to adopt the optimal position for the nucleophilic attack. In the computed model of the GnT I-Transition state complex (FIG. 10), the oligosaccharide-acceptor binds into a canal formed by a patch of hydrophobic residues (Phe-181, Tyr-184, Phe-289 and His-178). The key trimannosyl residues of the oligosaccharide-acceptor dock onto this region to stabilize the enzyme-transition state complex.

This example describes results on the catalytic mechanism and the homology modeling of GnTs and their complexes with ligands. These results contribute to the understanding of the reaction mechanism catalyzed by GnTs and to the essential requirements for a rational design of inhibitors. The results are consistent with the experimental data.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

(1) Beyer, T. A.; Sadler, J. E.; Rearick, J. I.; Paulson, J. C.; Hill, R. L. Adv. Enzymol. 1981, 52,23-175.

(2) Schachter, H. Curr. Opin. Struct. Biol. 1991, 1, 755-765.

(3) Kleene, R.; Berger, E. G. Biochim. Biophys. Acta 1993, 1154, 283-325.

(4) Montreuil, J.; Vliegenthart, J. F. G.; Schachter, H. Glycoproteins; Neuberger, A. and van Deenen, L. L. M., Ed.; Elsevier: Amsterdam, 1995; Vol. 29a.

(5) Montreuil, J.; Vliegenthart, J. F. G.; Schachter, H. Glycoproteins and Disease; Neuberger, A. and van Deenen, L. L. M., Ed.; Elsevier: Amsterdam, 1995; Vol.30.

(6) Bendiak, B.; Schachter, H. J Biol. Chem. 1987, 262, 5784-5790.

(7) Raju, T. S.; Stanley, P. J Biol. Chem. 1998, 273, 14090-14098.

(8) Vrielink, A.; Ruger, W.; Driessen, H. P. C.; Freemont, P. S. EMBO J. 1994, 13,3413-3422.

(9) Campbell, J. A.; Davies, G. J.; Bulone, V.; Henrissat, B. Biochem. J. 1997, 326, 929-942.

(10) Imberty, A.; Monier, C.; Bettler, E.; Morera, S.; Freemont, P.; Sippl, M.; Flockner, H.; Ruger, W.; Breton, C. Glycobiology 1999,9, 713-722.

Chamrock, S. J.; Davies, G. J. Biochemistry 1999, 38, 6380-6385.

(12) Gastinel, L. N.; Cambillau, C.; Boume, Y. EMBO J. 1999, 18, 3546-3557.

(13) Coutinho, P. M.; Henrissat, B.; http://afmb.cnrs-nirs.frl-pedro/CAZY/db.html.]

(14) Sinnott, M. L. Chem. Rev. 1990, 90, 1171-1202.

(15) Legler, G. Carbohydr. Res. 1993, 250, vii-xx.

(16) Davies, G.; Henrissat, B. Structure 1995, 3, 853-859.

(17) Withers, S. G.; Aebersold, R. Protein Sci. 1995, 4,361-372.

(18) Davies, G.; Sinnott, M. L.; Withers, S. G. Glycosyl transfer, Chapter 3; In Comprehensive Biological Catalysis; Sinott, M. L., Ed.; Academic Press Limited: New York, 1997, pp 119-208.

(19) Zechel, D. L.; Withers, S. G. Mechanisms of glycosyl transfer, Chapter 5; In Comprehensive Natural Products Chemistry; Poulter, C. D., Ed.; Elsevier: New York, 1999, pp 279-314.

(20) Withers, S. G.; Namchuk, M.; Mosi, R. Iminosugars as glycosidase inhibitors; Stutz, A. E., Ed.; Wiley-VCH: Weinheim, 1999, pp 188-206.

(21) Heightman, T. D.; Vasella, A. T. Angew. Chem. Int. Ed. 1999, 38,750-770.

(22) Nishikawa, Y.; Pegg, W.; Paulsen, H.; Schachter, H. J. Biol. Chem. 1988, 263, 8270-8281.

(23) Tvaroska, I.; André, I.; Carver, J. P. J. Mol. Struct. (77Theochem) 1999, 469, 103-114.

(24) Tvaroska, I.; André, I.; Carver, J. P. J. Phys. Chem.B 1999, 103, 2560-2569.

(25) André, I.; Tvaroska, I.; Carver, J. P. J. Phys. Chem. 1999, submitted

(26) Jaguar 3.5, S., Inc., Portland, Oreg., 1998.

(27) Becke, A. D. J Chem. Phys. 1993, 98, 5648-5652.

(28) Khatra, B. S.; Herries, D. G.; Brew, K. Eur. J. Biochem. 1974, 44,537-560.

(29) Warshel, A.; Weiss, R. M. J. Am. Chem. Soc. 1980, 102, 6218-6226.

(30) Sidhu, G.; Withers, S. G.; Nguyen, N. T.; McIntosh, L. P.; Ziser, L.; Brayer, G. D. Biochenmistry 1999, 38, 5346-5354.

(31) Brameld, K. A.; Goddard III, W. A. J. Am. Chem. Soc. 1998, 120,3571-3580.

(32) Fowler, P.; Bernet, B.; Vasella, A. Helv. Chim. Acta 1996, 79, 269-287.

(33) Sulzenbacher, G.; Driguez, H.; Henrissat, B.; Schulein, M.; Davies, G. J. Biochemistry 1996, 35, 15280-15287.

(34) Guthrie, J. P. J. Am. Chiem. Soc. 1977, 99, 3391-4000.

(35) Warshel, A. Computer modeling of Chemical Reactions in Enzymes and Solutions; John Wiley & Sons, Inc.: New York, 1997.

(36) Smith, B. J. J. Am. Chem. Soc. 1997, 119, 2699-2706.

(37) Sinnott, M. L. Adv. Phys. Org. Chem. 1988, 24, 113-224.

(38) Strynadka, N. C. J.; James, M. N. G. J. Mol. Biol. 1991, 220,401424.

(39) Zhang, N.; Peng, K. C.; Chen, L.; Puett, D.; Pierce, M. J. Biol. Chem. 1997, 272, 4225-4229.

(40) Murray, B. W.; Takayama, S.; Schultz, J.; Wong, C. -H. Biochemistry 1996, 35, 11183-11195.

(41) Murray, B. W.; Wittmann, V.; Burkart, M. D.; Hung, S. C.; Wong, C. -H. Biochemistry 1997, 36, 823-831.

(42) Seto, N. O.; Compston, C. A.; Evans, S. V.; Bundle, D. R.; Narang, S. A.; Palcic, M. M. Eur. J Biochein. 1999, 259, 770-775.

(43) Lu, D.; Voth, G. A. J. Am. Chem. Soc. 1998, 120, 4006-4014.

(44) Fuxreiter, M.; Warshel, A. J. Am. Chem. Soc. 1998, 120, 183-194.

(45) Kapitonov, D.; Yu, R. K. Glycobiology 1999, 9, 961-78.

(46) Hagen, F. K.; Hazes, B.; Raffo, R.; deSa, D.; Tabak, L. A. J. Biol. Chem. 1999, 274,6797-6803.

(47) Lu, P. P.; Hindsgaul, O.; Li, H.; Palcic, M. M. Carbohydr. Res. 1997,303,283-291.

48) Taguchi, T.; Kitajima, K.; Inoue, S.; Inoue, Y.; Yang, J. M.; Schachter, H.; Brockhausen, I. Biochem. Biophys. Res. Commun 1997, 230,533-6.

49) Unligil, U. M.; Zhou, S.; Yuwaraj, S.; Sarkar, M.; Schachter, H.; Rini, J. M. X-ray Crystal Structure of Rabbit N-Acetylglucosaminyltransferase I, a Key Enymine in the Biosynthesis of N-Linked Glycans: Annual Conference of the Society for Glycobiology. San Francisco, Calif., USA, 1999.

50) Meeting on the Critical Assessment of Techniques for Protein Structure Prediction (CASP) at URL: http://moult.carb.nist.gov/casp/.

51) Fischer, D.; Eisenberg, D. Protein Sci. 1996, 5, 947-55.

52) Fischer, D. Fold-Recognition server at URL: http./fold.doe-mbi.ucla.edu/Home.

53) Protein Data Bank at URL: http//www.rcsb.org/pdb/.

54) Needleman, S. B.; Wunch, C. D. J. Mol. Biol. 1970,48,443-453.

55) Homology 95.0 User Guide; Biosym/MSI: San Diego, 1995.

56) Goodsell, D. S.; Olson, A. J. Proteins 1990,8, 195-202.

TABLE 1
REMARK

| | | | | | | | | | | | | |
|---|---|---|---|---|---|---|---|---|---|---|---|---|
| ATOM | 1 | CB | ALA | 106A | −17.148 | −1.078 | 17.481 | 1.00 | 0.00 | −0.159 | 16.15 | 4.00 |
| ATOM | 2 | HB1 | ALA | 106A | −16.722 | −0.081 | 17.603 | 1.00 | 0.00 | 0.053 | 0.00 | 0.00 |
| ATOM | 3 | HB2 | ALA | 106A | −17.977 | −1.206 | 18.176 | 1.00 | 0.00 | 0.053 | 0.00 | 0.00 |
| ATOM | 4 | HB3 | ALA | 106A | −16.383 | −1.826 | 17.686 | 1.00 | 0.00 | 0.053 | 0.00 | 0.00 |
| ATOM | 5 | C | ALA | 106A | −16.506 | −1.033 | 15.073 | 1.00 | 0.00 | 0.396 | 9.82 | 4.00 |
| ATOM | 6 | O | ALA | 106A | −15.407 | −1.538 | 15.276 | 1.00 | 0.00 | −0.396 | 8.17 | −17.40 |
| ATOM | 7 | N | ALA | 106A | −18.246 | −2.612 | 15.849 | 1.00 | 0.00 | −0.580 | 9.00 | −17.40 |
| ATOM | 8 | HN1 | ALA | 106A | −19.264 | −2.530 | 15.721 | 1.00 | 0.00 | 0.249 | 0.00 | 0.00 |
| ATOM | 9 | HN2 | ALA | 106A | −17.830 | −3.050 | 15.015 | 1.00 | 0.00 | 0.249 | 0.00 | 0.00 |
| ATOM | 10 | CA | ALA | 106A | −17.654 | −1.245 | 16.045 | 1.00 | 0.00 | 0.030 | 9.40 | 4.00 |
| ATOM | 11 | HA | ALA | 106A | −18.412 | −0.482 | 15.869 | 1.00 | 0.00 | 0.053 | 0.00 | 0.00 |
| ATOM | 12 | N | VAL | 107 | −16.765 | −0.282 | 14.012 | 1.00 | 0.00 | −0.650 | 9.00 | −17.40 |
| ATOM | 13 | HN | VAL | 107 | −17.706 | 0.118 | 13.890 | 1.00 | 0.00 | 0.440 | 0.00 | 0.00 |
| ATOM | 14 | CA | VAL | 107 | −15.742 | −0.016 | 13.021 | 1.00 | 0.00 | 0.158 | 9.40 | 4.00 |
| ATOM | 15 | HA | VAL | 107 | −15.072 | −0.874 | 12.966 | 1.00 | 0.00 | 0.053 | 0.00 | 0.00 |
| ATOM | 16 | CB | VAL | 107 | −16.385 | 0.191 | 11.638 | 1.00 | 0.00 | −0.053 | 9.40 | 4.00 |
| ATOM | 17 | HB | VAL | 107 | −17.143 | 0.972 | 11.685 | 1.00 | 0.00 | 0.053 | 0.00 | 0.00 |
| ATOM | 18 | CG1 | VAL | 107 | −15.336 | 0.599 | 10.624 | 1.00 | 0.00 | −0.159 | 16.15 | 4.00 |
| ATOM | 19 | HG1 | VAL | 107 | −15.806 | 0.741 | 9.651 | 1.00 | 0.00 | 0.053 | 0.00 | 0.00 |
| ATOM | 20 | HG1 | VAL | 107 | −14.867 | 1.530 | 10.940 | 1.00 | 0.00 | 0.053 | 0.00 | 0.00 |
| ATOM | 21 | HG1 | VAL | 107 | −14.578 | −0.181 | 10.550 | 1.00 | 0.00 | 0.053 | 0.00 | 0.00 |
| ATOM | 22 | CG2 | VAL | 107 | −17.075 | −1.098 | 11.206 | 1.00 | 0.00 | −0.159 | 16.15 | 4.00 |
| ATOM | 23 | HG2 | VAL | 107 | −17.531 | −0.955 | 10.226 | 1.00 | 0.00 | 0.053 | 0.00 | 0.00 |
| ATOM | 24 | HG2 | VAL | 107 | −16.341 | −1.902 | 11.150 | 1.00 | 0.00 | 0.053 | 0.00 | 0.00 |
| ATOM | 25 | HG2 | VAL | 107 | −17.845 | −1.358 | 11.931 | 1.00 | 0.00 | 0.053 | 0.00 | 0.00 |
| ATOM | 26 | C | VAL | 107 | −14.964 | 1.229 | 13.437 | 1.00 | 0.00 | 0.396 | 9.82 | 4.00 |
| ATOM | 27 | O | VAL | 107 | −15.559 | 2.286 | 13.678 | 1.00 | 0.00 | −0.396 | 8.17 | −17.40 |
| ATOM | 28 | N | ILE | 108 | −13.641 | 1.098 | 13.523 | 1.00 | 0.00 | −0.650 | 9.00 | −17.40 |
| ATOM | 29 | HN | ILE | 108 | −13.215 | 0.185 | 13.304 | 1.00 | 0.00 | 0.440 | 0.00 | 0.00 |
| ATOM | 30 | CA | ILE | 108 | −12.772 | 2.209 | 13.918 | 1.00 | 0.00 | 0.158 | 9.40 | 4.00 |
| ATOM | 31 | HA | ILE | 108 | −13.380 | 3.076 | 14.173 | 1.00 | 0.00 | 0.053 | 0.00 | 0.00 |
| ATOM | 32 | CB | ILE | 108 | −11.894 | 1.841 | 15.128 | 1.00 | 0.00 | −0.053 | 9.40 | 4.00 |
| ATOM | 33 | HB | ILE | 108 | −11.260 | 0.989 | 14.880 | 1.00 | 0.00 | 0.053 | 0.00 | 0.00 |
| ATOM | 34 | CG2 | ILE | 108 | −11.002 | 3.039 | 15.511 | 1.00 | 0.00 | −0.159 | 16.15 | 4.00 |
| ATOM | 35 | HG2 | ILE | 108 | −10.383 | 2.772 | 16.367 | 1.00 | 0.00 | 0.053 | 0.00 | 0.00 |
| ATOM | 36 | HG2 | ILE | 108 | −10.361 | 3.300 | 14.668 | 1.00 | 0.00 | 0.053 | 0.00 | 0.00 |
| ATOM | 37 | HG2 | ILE | 108 | −11.629 | 3.892 | 15.768 | 1.00 | 0.00 | 0.053 | 0.00 | 0.00 |
| ATOM | 38 | CG1 | ILE | 108 | −12.776 | 1.453 | 16.315 | 1.00 | 0.00 | −0.106 | 12.77 | 4.00 |
| ATOM | 39 | HG1 | ILE | 108 | −13.473 | 0.649 | 16.081 | 1.00 | 0.00 | 0.053 | 0.00 | 0.00 |
| ATOM | 40 | HG1 | ILE | 108 | −13.387 | 2.279 | 16.678 | 1.00 | 0.00 | 0.053 | 0.00 | 0.00 |
| ATOM | 41 | CDl | ILE | 108 | −11.971 | 0.967 | 17.524 | 1.00 | 0.00 | −0.159 | 16.15 | 4.00 |
| ATOM | 42 | HD1 | ILE | 108 | −12.652 | 0.706 | 18.334 | 1.00 | 0.00 | 0.053 | 0.00 | 0.00 |
| ATOM | 43 | HD1 | ILE | 108 | −11.387 | 0.089 | 17.244 | 1.00 | 0.00 | 0.053 | 0.00 | 0.00 |
| ATOM | 44 | HD1 | ILE | 108 | −11.299 | 1.758 | 17.855 | 1.00 | 0.00 | 0.053 | 0.00 | 0.00 |
| ATOM | 45 | C | ILE | 108 | −11.845 | 2.565 | 12.767 | 1.00 | 0.00 | 0.396 | 9.82 | 4.00 |
| ATOM | 46 | O | ILE | 108 | −10.877 | 1.862 | 12.498 | 1.00 | 0.00 | −0.396 | 8.17 | −17.40 |
| ATOM | 47 | N | PRO | 109 | −12.135 | 3.670 | 12.073 | 1.00 | 0.00 | −0.422 | 9.00 | −17.40 |
| ATOM | 48 | CD | PRO | 109 | −13.304 | 4.556 | 12.253 | 1.00 | 0.00 | 0.105 | 12.77 | 4.00 |
| ATOM | 49 | HD1 | PRO | 109 | −13.420 | 4.849 | 13.296 | 1.00 | 0.00 | 0.053 | 0.00 | 0.00 |
| ATOM | 50 | HD2 | PRO | 109 | −14.225 | 4.062 | 11.944 | 1.00 | 0.00 | 0.053 | 0.00 | 0.00 |
| ATOM | 51 | CA | PRO | 109 | −11.316 | 4.106 | 10.944 | 1.00 | 0.00 | 0.158 | 9.40 | 4.00 |
| ATOM | 52 | HA | PRO | 109 | −11.017 | 3.286 | 10.290 | 1.00 | 0.00 | 0.053 | 0.00 | 0.00 |
| ATOM | 53 | CB | PRO | 109 | −12.236 | 5.070 | 10.215 | 1.00 | 0.00 | −0.106 | 12.77 | 4.00 |
| ATOM | 54 | HB1 | PRO | 109 | −12.913 | 4.543 | 9.543 | 1.00 | 0.00 | 0.053 | 0.00 | 0.00 |
| ATOM | 55 | HB2 | PRO | 109 | −11.671 | 5.783 | 9.615 | 1.00 | 0.00 | 0.053 | 0.00 | 0.00 |
| ATOM | 56 | CG | PRO | 109 | −12.961 | 5.730 | 11.366 | 1.00 | 0.00 | −0.106 | 12.77 | 4.00 |
| ATOM | 57 | HG1 | PRO | 109 | −13.850 | 6.257 | 11.022 | 1.00 | 0.00 | 0.053 | 0.00 | 0.00 |
| ATOM | 58 | HG2 | PRO | 109 | −12.322 | 6.453 | 11.872 | 1.00 | 0.00 | 0.053 | 0.00 | 0.00 |
| ATOM | 59 | C | PRO | 109 | −10.025 | 4.780 | 11.370 | 1.00 | 0.00 | 0.396 | 9.82 | 4.00 |
| ATOM | 60 | O | PRO | 109 | −9.933 | 5.391 | 12.450 | 1.00 | 0.00 | −0.396 | 8.17 | −17.40 |
| ATOM | 61 | N | ILE | 110 | −9.010 | 4.618 | 10.532 | 1.00 | 0.00 | −0.650 | 9.00 | −17.40 |
| ATOM | 62 | HN | ILE | 110 | −9.128 | 4.021 | 9.700 | 1.00 | 0.00 | 0.440 | 0.00 | 0.00 |
| ATOM | 63 | CA | ILE | 110 | −7.740 | 5.266 | 10.768 | 1.00 | 0.00 | 0.158 | 9.40 | 4.00 |
| ATOM | 64 | HA | ILE | 110 | −7.645 | 5.602 | 11.800 | 1.00 | 0.00 | 0.053 | 0.00 | 0.00 |
| ATOM | 65 | CB | ILE | 110 | −6.542 | 4.379 | 10.376 | 1.00 | 0.00 | −0.053 | 9.40 | 4.00 |
| ATOM | 66 | HB | ILE | 110 | −6.559 | 4.187 | 9.303 | 1.00 | 0.00 | 0.053 | 0.00 | 0.00 |
| ATOM | 67 | CG2 | ILE | 110 | −5.217 | 5.093 | 10.742 | 1.00 | 0.00 | −0.159 | 16.15 | 4.00 |
| ATOM | 68 | HG2 | ILE | 110 | −4.373 | 4.461 | 10.462 | 1.00 | 0.00 | 0.053 | 0.00 | 0.00 |
| ATOM | 69 | HG2 | ILE | 110 | −5.153 | 6.040 | 10.206 | 1.00 | 0.00 | 0.053 | 0.00 | 0.00 |
| ATOM | 70 | HG2 | ILE | 110 | −5.189 | 5.280 | 11.815 | 1.00 | 0.00 | 0.053 | 0.00 | 0.00 |
| ATOM | 71 | CG1 | ILE | 110 | −6.673 | 3.009 | 11.041 | 1.00 | 0.00 | −0.106 | 12.77 | 4.00 |
| ATOM | 72 | HG1 | ILE | 110 | −5.827 | 2.397 | 10.726 | 1.00 | 0.00 | 0.053 | 0.00 | 0.00 |
| ATOM | 73 | HG1 | ILE | 110 | −7.614 | 2.564 | 10.717 | 1.00 | 0.00 | 0.053 | 0.00 | 0.00 |
| ATOM | 74 | CD1 | ILE | 110 | −6.679 | 3.028 | 12.571 | 1.00 | 0.00 | −0.159 | 16.15 | 4.00 |
| ATOM | 75 | HD1 | ILE | 110 | −6.775 | 2.009 | 12.947 | 1.00 | 0.00 | 0.053 | 0.00 | 0.00 |
| ATOM | 76 | HD1 | ILE | 110 | −5.746 | 3.461 | 12.933 | 1.00 | 0.00 | 0.053 | 0.00 | 0.00 |
| ATOM | 77 | HD1 | ILE | 110 | −7.518 | 3.626 | 12.924 | 1.00 | 0.00 | 0.053 | 0.00 | 0.00 |
| ATOM | 78 | C | ILE | 110 | −7.821 | 6.417 | 9.778 | 1.00 | 0.00 | 0.396 | 9.82 | 4.00 |
| ATOM | 79 | O | ILE | 110 | −7.960 | 6.185 | 8.568 | 1.00 | 0.00 | −0.396 | 8.17 | −17.40 |
| ATOM | 80 | N | LEU | 111 | −7.772 | 7.644 | 10.288 | 1.00 | 0.00 | −0.650 | 9.00 | −17.40 |
| ATOM | 81 | HN | LEU | 111 | −7.701 | 7.766 | 11.308 | 1.00 | 0.00 | 0.440 | 0.00 | 0.00 |
| ATOM | 82 | CA | LEU | 111 | −7.817 | 8.815 | 9.429 | 1.00 | 0.00 | 0.158 | 9.40 | 4.00 |
| ATOM | 83 | HA | LEU | 111 | −8.247 | 8.489 | 8.482 | 1.00 | 0.00 | 0.053 | 0.00 | 0.00 |
| ATOM | 84 | CB | LEU | 111 | −8.659 | 9.929 | 10.051 | 1.00 | 0.00 | −0.106 | 12.77 | 4.00 |
| ATOM | 85 | HB1 | LEU | 111 | −8.370 | 10.024 | 11.098 | 1.00 | 0.00 | 0.053 | 0.00 | 0.00 |
| ATOM | 86 | HB2 | LEU | 111 | −9.708 | 9.648 | 9.960 | 1.00 | 0.00 | 0.053 | 0.00 | 0.00 |
| ATOM | 87 | CG | LEU | 111 | −8.502 | 11.311 | 9.410 | 1.00 | 0.00 | −0.053 | 9.40 | 4.00 |
| ATOM | 88 | HG | LEU | 111 | −7.462 | 11.633 | 9.457 | 1.00 | 0.00 | 0.053 | 0.00 | 0.00 |
| ATOM | 89 | CD1 | LEU | 111 | −8.934 | 11.268 | 7.944 | 1.00 | 0.00 | −0.159 | 16.15 | 4.00 |
| ATOM | 90 | HD1 | LEU | 111 | −8.817 | 12.257 | 7.501 | 1.00 | 0.00 | 0.053 | 0.00 | 0.00 |
| ATOM | 91 | HD1 | LEU | 111 | −8.314 | 10.552 | 7.403 | 1.00 | 0.00 | 0.053 | 0.00 | 0.00 |
| ATOM | 92 | HD1 | LEU | 111 | −9.978 | 10.963 | 7.881 | 1.00 | 0.00 | 0.053 | 0.00 | 0.00 |
| ATOM | 93 | CD2 | LEU | 111 | −9.351 | 12.310 | 10.180 | 1.00 | 0.00 | −0.159 | 16.15 | 4.00 |
| ATOM | 94 | HD2 | LEU | 111 | −9.246 | 13.298 | 9.731 | 1.00 | 0.00 | 0.053 | 0.00 | 0.00 |
| ATOM | 95 | HD2 | LEU | 111 | −10.396 | 12.003 | 10.143 | 1.00 | 0.00 | 0.053 | 0.00 | 0.00 |
| ATOM | 96 | HD2 | LEU | 111 | −9.019 | 12.345 | 11.217 | 1.00 | 0.00 | 0.053 | 0.00 | 0.00 |
| ATOM | 97 | C | LEU | 111 | −6.380 | 9.295 | 9.273 | 1.00 | 0.00 | 0.396 | 9.82 | 4.00 |
| ATOM | 98 | O | LEU | 111 | −5.758 | 9.752 | 10.230 | 1.00 | 0.00 | −0.396 | 8.17 | −17.40 |
| ATOM | 99 | N | VAL | 112 | −5.854 | 9.151 | 8.068 | 1.00 | 0.00 | −0.650 | 9.00 | −17.40 |
| ATOM | 100 | HN | VAL | 112 | −6.417 | 8.712 | 7.325 | 1.00 | 0.00 | 0.440 | 0.00 | 0.00 |
| ATOM | 101 | CA | VAL | 112 | −4.503 | 9.594 | 7.762 | 1.00 | 0.00 | 0.158 | 9.40 | 4.00 |
| ATOM | 102 | HA | VAL | 112 | −3.905 | 9.594 | 8.673 | 1.00 | 0.00 | 0.053 | 0.00 | 0.00 |
| ATOM | 103 | CB | VAL | 112 | −3.834 | 8.632 | 6.751 | 1.00 | 0.00 | −0.053 | 9.40 | 4.00 |
| ATOM | 104 | HB | VAL | 112 | −4.445 | 8.521 | 5.855 | 1.00 | 0.00 | 0.053 | 0.00 | 0.00 |
| ATOM | 105 | CG1 | VAL | 112 | −2.476 | 9.150 | 6.329 | 1.00 | 0.00 | −0.159 | 16.15 | 4.00 |
| ATOM | 106 | HG1 | VAL | 112 | −2.026 | 8.456 | 5.618 | 1.00 | 0.00 | 0.053 | 0.00 | 0.00 |
| ATOM | 107 | HG1 | VAL | 112 | −2.589 | 10.127 | 5.859 | 1.00 | 0.00 | 0.053 | 0.00 | 0.00 |
| ATOM | 108 | HG1 | VAL | 112 | −1.832 | 9.240 | 7.204 | 1.00 | 0.00 | 0.053 | 0.00 | 0.00 |
| ATOM | 109 | CG2 | VAL | 112 | −3.702 | 7.236 | 7.371 | 1.00 | 0.00 | −0.159 | 16.15 | 4.00 |
| ATOM | 110 | HG2 | VAL | 112 | −3.230 | 6.562 | 6.655 | 1.00 | 0.00 | 0.053 | 0.00 | 0.00 |
| ATOM | 111 | HG2 | VAL | 112 | −3.090 | 7.294 | 8.271 | 1.00 | 0.00 | 0.053 | 0.00 | 0.00 |
| ATOM | 112 | HG2 | VAL | 112 | −4.691 | 6.857 | 7.628 | 1.00 | 0.00 | 0.053 | 0.00 | 0.00 |
| ATOM | 113 | C | VAL | 112 | −4.549 | 11.016 | 7.179 | 1.00 | 0.00 | 0.396 | 9.82 | 4.00 |
| ATOM | 114 | O | VAL | 112 | −5.245 | 11.273 | 6.188 | 1.00 | 0.00 | −0.396 | 8.17 | −17.40 |
| ATOM | 115 | N | ILE | 113 | −3.802 | 11.924 | 7.806 | 1.00 | 0.00 | −0.650 | 9.00 | −17.40 |
| ATOM | 116 | HN | ILE | 113 | −3.260 | 11.626 | 8.630 | 1.00 | 0.00 | 0.440 | 0.00 | 0.00 |
| ATOM | 117 | CA | ILE | 113 | −3.713 | 13.318 | 7.384 | 1.00 | 0.00 | 0.158 | 9.40 | 4.00 |
| ATOM | 118 | HA | ILE | 113 | −4.610 | 13.526 | 6.801 | 1.00 | 0.00 | 0.053 | 0.00 | 0.00 |
| ATOM | 119 | CB | ILE | 113 | −3.687 | 14.304 | 8.604 | 1.00 | 0.00 | −0.053 | 9.40 | 4.00 |
| ATOM | 120 | HB | ILE | 113 | −2.859 | 14.067 | 9.272 | 1.00 | 0.00 | 0.053 | 0.00 | 0.00 |
| ATOM | 121 | CG2 | ILE | 113 | −3.520 | 15.752 | 8.126 | 1.00 | 0.00 | −0.159 | 16.15 | 4.00 |
| ATOM | 122 | HG2 | ILE | 113 | −3.504 | 16.419 | 8.987 | 1.00 | 0.00 | 0.053 | 0.00 | 0.00 |
| ATOM | 123 | HG2 | ILE | 113 | −2.584 | 15.847 | 7.575 | 1.00 | 0.00 | 0.053 | 0.00 | 0.00 |
| ATOM | 124 | HG2 | ILE | 113 | −4.352 | 16.018 | 7.475 | 1.00 | 0.00 | 0.053 | 0.00 | 0.00 |
| ATOM | 125 | CG1 | ILE | 113 | −5.014 | 14.224 | 9.377 | 1.00 | 0.00 | −0.106 | 12.77 | 4.00 |
| ATOM | 126 | HG1 | ILE | 113 | −5.892 | 14.359 | 8.746 | 1.00 | 0.00 | 0.053 | 0.00 | 0.00 |
| ATOM | 127 | HG1 | ILE | 113 | −5.105 | 14.976 | 10.160 | 1.00 | 0.00 | 0.053 | 0.00 | 0.00 |
| ATOM | 128 | CD1 | ILE | 113 | −5.223 | 12.918 | 10.063 | 1.00 | 0.00 | −0.159 | 16.15 | 4.00 |
| ATOM | 129 | HD1 | ILE | 113 | −6.178 | 12.931 | 10.588 | 1.00 | 0.00 | 0.053 | 0.00 | 0.00 |
| ATOM | 130 | HD1 | ILE | 113 | −5.226 | 12.115 | 9.325 | 1.00 | 0.00 | 0.053 | 0.00 | 0.00 |
| ATOM | 131 | HD1 | ILE | 113 | −4.417 | 12.749 | 10.778 | 1.00 | 0.00 | 0.053 | 0.00 | 0.00 |
| ATOM | 132 | C | ILE | 113 | −2.445 | 13.442 | 6.549 | 1.00 | 0.00 | 0.396 | 9.82 | 4.00 |
| ATOM | 133 | O | ILE | 113 | −1.349 | 13.096 | 7.003 | 1.00 | 0.00 | −0.396 | 8.17 | −17.40 |
| ATOM | 134 | N | ALA | 114 | −2.619 | 13.927 | 5.320 | 1.00 | 0.00 | −0.650 | 9.00 | −17.40 |
| ATOM | 135 | HN | ALA | 114 | −3.564 | 14.220 | 5.035 | 1.00 | 0.00 | 0.440 | 0.00 | 0.00 |
| ATOM | 136 | CA | ALA | 114 | −1.526 | 14.061 | 4.358 | 1.00 | 0.00 | 0.158 | 9.40 | 4.00 |
| ATOM | 137 | HA | ALA | 114 | −0.556 | 13.990 | 4.851 | 1.00 | 0.00 | 0.053 | 0.00 | 0.00 |
| ATOM | 138 | CB | ALA | 114 | −1.616 | 12.899 | 3.319 | 1.00 | 0.00 | −0.159 | 16.15 | 4.00 |
| ATOM | 139 | HB1 | ALA | 114 | −0.803 | 12.992 | 2.598 | 1.00 | 0.00 | 0.053 | 0.00 | 0.00 |
| ATOM | 140 | HB2 | ALA | 114 | −1.535 | 11.942 | 3.835 | 1.00 | 0.00 | 0.053 | 0.00 | 0.00 |
| ATOM | 141 | HB3 | ALA | 114 | −2.571 | 12.950 | 2.797 | 1.00 | 0.00 | 0.053 | 0.00 | 0.00 |
| ATOM | 142 | C | ALA | 114 | −1.585 | 15.410 | 3.638 | 1.00 | 0.00 | 0.396 | 9.82 | 4.00 |
| ATOM | 143 | O | ALA | 114 | −2.595 | 16.112 | 3.705 | 1.00 | 0.00 | −0.396 | 8.17 | −17.40 |
| ATOM | 144 | N | CYS | 115 | −0.504 | 15.768 | 2.938 | 1.00 | 0.00 | −0.650 | 9.00 | −17.40 |
| ATOM | 145 | HN | CYS | 115 | 0.311 | 15.139 | 2.908 | 1.00 | 0.00 | 0.440 | 0.00 | 0.00 |
| ATOM | 146 | CA | CYS | 115 | −0.459 | 17.042 | 2.211 | 1.00 | 0.00 | 0.158 | 9.40 | 4.00 |
| ATOM | 147 | HA | CYS | 115 | −1.428 | 17.348 | 1.819 | 1.00 | 0.00 | 0.053 | 0.00 | 0.00 |
| ATOM | 148 | C | CYS | 115 | 0.474 | 17.015 | 1.007 | 1.00 | 0.00 | 0.396 | 9.82 | 4.00 |
| ATOM | 149 | O | CYS | 115 | 0.035 | 16.810 | −0.139 | 1.00 | 0.00 | −0.396 | 8.17 | −17.40 |
| ATOM | 150 | CB | CYS | 115 | −0.048 | 18.162 | 3.182 | 1.00 | 0.00 | −0.041 | 12.77 | 4.00 |
| ATOM | 151 | HB1 | CYS | 115 | 0.803 | 17.788 | 3.751 | 1.00 | 0.00 | 0.053 | 0.00 | 0.00 |
| ATOM | 152 | HB2 | CYS | 115 | −0.910 | 18.359 | 3.818 | 1.00 | 0.00 | 0.053 | 0.00 | 0.00 |
| ATOM | 153 | SG | CYS | 115 | 0.470 | 19.793 | 2.539 | 1.00 | 0.00 | −0.065 | 19.93 | −6.40 |
| ATOM | 154 | N | ASP | 116P | 1.766 | 17.200 | 1.268 | 1.00 | 0.00 | −0.650 | 9.00 | −17.40 |
| ATOM | 155 | HN | ASP | 116P | 2.079 | 17.312 | 2.242 | 1.00 | 0.00 | 0.440 | 0.00 | 0.00 |
| ATOM | 156 | CA | ASP | 116P | 2.737 | 17.245 | 0.189 | 1.00 | 0.00 | 0.158 | 9.40 | 4.00 |
| ATOM | 157 | HA | ASP | 116P | 2.249 | 16.867 | −0.709 | 1.00 | 0.00 | 0.053 | 0.00 | 0.00 |
| ATOM | 158 | CB | ASP | 116P | 3.111 | 18.711 | −0.106 | 1.00 | 0.00 | −0.336 | 12.77 | 4.00 |
| ATOM | 159 | HB1 | ASP | 116P | 2.257 | 19.320 | −0.403 | 1.00 | 0.00 | 0.053 | 0.00 | 0.00 |
| ATOM | 160 | HB2 | ASP | 116P | 3.836 | 18.814 | −0.913 | 1.00 | 0.00 | 0.053 | 0.00 | 0.00 |
| ATOM | 161 | CG | ASP | 116P | 3.729 | 19.429 | 1.093 | 1.00 | 0.00 | 0.297 | 9.82 | 4.00 |
| ATOM | 162 | OD1 | ASP | 116P | 3.864 | 20.668 | 1.027 | 1.00 | 0.00 | −0.534 | 8.17 | −18.95 |
| ATOM | 163 | OD2 | ASP | 116P | 4.093 | 18.779 | 2.096 | 1.00 | 0.00 | −0.534 | 8.17 | −18.95 |
| ATOM | 164 | C | ASP | 116P | 4.003 | 16.435 | 0.371 | 1.00 | 0.00 | 0.396 | 9.82 | 4.00 |
| ATOM | 165 | O | ASP | 116P | 5.038 | 16.772 | −0.228 | 1.00 | 0.00 | −0.396 | 8.17 | −17.40 |
| ATOM | 166 | N | ARG | 117G | 3.943 | 15.390 | 1.198 | 1.00 | 0.00 | −0.650 | 9.00 | −17.40 |
| ATOM | 167 | HN | ARG | 117G | 3.065 | 15.189 | 1.697 | 1.00 | 0.00 | 0.440 | 0.00 | 0.00 |
| ATOM | 168 | CA | ARG | 117G | 5.101 | 14.527 | 1.406 | 1.00 | 0.00 | 0.158 | 9.40 | 4.00 |
| ATOM | 169 | HA | ARG | 117G | 5.955 | 14.953 | 0.879 | 1.00 | 0.00 | 0.053 | 0.00 | 0.00 |
| ATOM | 170 | CB | ARG | 117G | 5.454 | 14.440 | 2.905 | 1.00 | 0.00 | −0.106 | 12.77 | 4.00 |
| ATOM | 171 | HB1 | ARG | 117G | 6.128 | 13.617 | 3.144 | 1.00 | 0.00 | 0.053 | 0.00 | 0.00 |
| ATOM | 172 | HB2 | ARG | 117G | 4.585 | 14.294 | 3.547 | 1.00 | 0.00 | 0.053 | 0.00 | 0.00 |
| ATOM | 173 | CG | ARG | 117G | 6.149 | 15.705 | 3.445 | 1.00 | 0.00 | −0.106 | 12.77 | 4.00 |
| ATOM | 174 | HG1 | ARG | 117G | 5.526 | 16.597 | 3.384 | 1.00 | 0.00 | 0.053 | 0.00 | 0.00 |
| ATOM | 175 | HG2 | ARG | 117G | 7.065 | 15.953 | 2.910 | 1.00 | 0.00 | 0.053 | 0.00 | 0.00 |
| ATOM | 176 | CD | ARG | 117G | 6.560 | 15.589 | 4.939 | 1.00 | 0.00 | 0.374 | 12.77 | 4.00 |
| ATOM | 177 | HD1 | ARG | 117G | 7.067 | 16.479 | 5.310 | 1.00 | 0.00 | 0.053 | 0.00 | 0.00 |
| ATOM | 178 | HD2 | ARG | 117G | 7.240 | 14.758 | 5.128 | 1.00 | 0.00 | 0.053 | 0.00 | 0.00 |
| ATOM | 179 | NE | ARG | 117G | 5.422 | 15.381 | 5.826 | 1.00 | 0.00 | −0.819 | 9.00 | −24.67 |
| ATOM | 180 | HE | ARG | 117G | 4.617 | 14.851 | 5.461 | 1.00 | 0.00 | 0.407 | 0.00 | 0.00 |
| ATOM | 181 | CZ | ARG | 117G | 5.354 | 15.831 | 7.077 | 1.00 | 0.00 | 0.796 | 6.95 | 4.00 |
| ATOM | 182 | NH1 | ARG | 117G | 6.376 | 16.534 | 7.595 | 1.00 | 0.00 | −0.746 | 9.00 | −24.67 |
| ATOM | 183 | HH1 | ARG | 117G | 6.323 | 16.882 | 8.563 | 1.00 | 0.00 | 0.407 | 0.00 | 0.00 |
| ATOM | 184 | HH1 | ARG | 117G | 7.211 | 16.724 | 7.024 | 1.00 | 0.00 | 0.407 | 0.00 | 0.00 |
| ATOM | 185 | NH2 | ARG | 117G | 4.267 | 15.584 | 7.808 | 1.00 | 0.00 | −0.746 | 9.00 | −24.67 |
| ATOM | 186 | HH2 | ARG | 117G | 4.208 | 15.929 | 8.776 | 1.00 | 0.00 | 0.407 | 0.00 | 0.00 |
| ATOM | 187 | HH2 | ARG | 117G | 3.485 | 15.047 | 7.404 | 1.00 | 0.00 | 0.407 | 0.00 | 0.00 |
| ATOM | 188 | C | ARG | 117G | 4.783 | 13.136 | 0.856 | 1.00 | 0.00 | 0.396 | 9.82 | 4.00 |
| ATOM | 189 | O | ARG | 117G | 3.832 | 12.475 | 1.306 | 1.00 | 0.00 | −0.396 | 8.17 | −17.40 |
| ATOM | 190 | N | SER | 118 | 5.581 | 12.692 | −0.111 | 1.00 | 0.00 | −0.650 | 9.00 | −17.40 |
| ATOM | 191 | HN | SER | 118 | 6.356 | 13.288 | −0.433 | 1.00 | 0.00 | 0.440 | 0.00 | 0.00 |
| ATOM | 192 | CA | SER | 118 | 5.390 | 11.388 | −0.728 | 1.00 | 0.00 | 0.158 | 9.40 | 4.00 |
| ATOM | 193 | HA | SER | 118 | 4.363 | 11.252 | −1.068 | 1.00 | 0.00 | 0.053 | 0.00 | 0.00 |
| ATOM | 194 | CB | SER | 118 | 6.266 | 11.235 | −1.983 | 1.00 | 0.00 | 0.007 | 12.77 | 4.00 |
| ATOM | 195 | HB1 | SER | 118 | 5.990 | 11.965 | −2.744 | 1.00 | 0.00 | 0.053 | 0.00 | 0.00 |
| ATOM | 196 | HB2 | SER | 118 | 6.156 | 10.241 | −2.418 | 1.00 | 0.00 | 0.053 | 0.00 | 0.00 |
| ATOM | 197 | OG | SER | 118 | 7.640 | 11.426 | −1.672 | 1.00 | 0.00 | −0.537 | 11.04 | −17.40 |
| ATOM | 198 | HG | SER | 118 | 8.102 | 11.920 | −2.449 | 1.00 | 0.00 | 0.424 | 0.00 | 0.00 |
| ATOM | 199 | C | SER | 118 | 5.696 | 10.269 | 0.236 | 1.00 | 0.00 | 0.396 | 9.82 | 4.00 |
| ATOM | 200 | O | SER | 118 | 5.278 | 9.133 | 0.007 | 1.00 | 0.00 | −0.396 | 8.17 | −17.40 |
| ATOM | 201 | N | THR | 119 | 6.421 | 10.588 | 1.304 | 1.00 | 0.00 | −0.650 | 9.00 | −17.40 |
| ATOM | 202 | HN | THR | 119 | 6.753 | 11.556 | 1.415 | 1.00 | 0.00 | 0.440 | 0.00 | 0.00 |
| ATOM | 203 | CA | THR | 119 | 6.756 | 9.602 | 2.318 | 1.00 | 0.00 | 0.158 | 9.40 | 4.00 |
| ATOM | 204 | HA | THR | 119 | 7.211 | 8.714 | 1.878 | 1.00 | 0.00 | 0.053 | 0.00 | 0.00 |
| ATOM | 205 | CB | THR | 119 | 7.826 | 10.147 | 3.300 | 1.00 | 0.00 | 0.060 | 9.40 | 4.00 |
| ATOM | 206 | HB | THR | 119 | 7.958 | 9.460 | 4.136 | 1.00 | 0.00 | 0.053 | 0.00 | 0.00 |
| ATOM | 207 | OG1 | THR | 119 | 7.413 | 11.418 | 3.813 | 1.00 | 0.00 | −0.537 | 11.04 | −17.40 |
| ATOM | 208 | HG1 | THR | 119 | 8.175 | 12.100 | 3.696 | 1.00 | 0.00 | 0.424 | 0.00 | 0.00 |
| ATOM | 209 | CG2 | THR | 119 | 9.167 | 10.310 | 2.589 | 1.00 | 0.00 | −0.159 | 16.15 | 4.00 |
| ATOM | 210 | HG2 | THR | 119 | 9.907 | 10.693 | 3.291 | 1.00 | 0.00 | 0.053 | 0.00 | 0.00 |
| ATOM | 211 | HG2 | THR | 119 | 9.495 | 9.343 | 2.206 | 1.00 | 0.00 | 0.053 | 0.00 | 0.00 |
| ATOM | 212 | HG2 | THR | 119 | 9.056 | 11.009 | 1.760 | 1.00 | 0.00 | 0.053 | 0.00 | 0.00 |
| ATOM | 213 | C | THR | 119 | 5.508 | 9.142 | 3.092 | 1.00 | 0.00 | 0.396 | 9.82 | 4.00 |
| ATOM | 214 | O | THR | 119 | 5.593 | 8.372 | 4.060 | 1.00 | 0.00 | −0.396 | 8.17 | −17.40 |
| ATOM | 215 | N | VAL | 120 | 4.342 | 9.636 | 2.685 | 1.00 | 0.00 | −0.650 | 9.00 | −17.40 |
| ATOM | 216 | HN | VAL | 120 | 4.310 | 10.347 | 1.941 | 1.00 | 0.00 | 0.440 | 0.00 | 0.00 |
| ATOM | 217 | CA | VAL | 120 | 3.117 | 9.164 | 3.297 | 1.00 | 0.00 | 0.158 | 9.40 | 4.00 |
| ATOM | 218 | HA | VAL | 120 | 3.126 | 9.327 | 4.374 | 1.00 | 0.00 | 0.053 | 0.00 | 0.00 |
| ATOM | 219 | CB | VAL | 120 | 1.871 | 9.876 | 2.703 | 1.00 | 0.00 | −0.053 | 9.40 | 4.00 |
| ATOM | 220 | HB | VAL | 120 | 1.914 | 10.944 | 2.914 | 1.00 | 0.00 | 0.053 | 0.00 | 0.00 |
| ATOM | 221 | CG1 | VAL | 120 | 1.823 | 9.673 | 1.187 | 1.00 | 0.00 | −0.159 | 16.15 | 4.00 |
| ATOM | 222 | HG1 | VAL | 120 | 0.945 | 10.176 | 0.780 | 1.00 | 0.00 | 0.053 | 0.00 | 0.00 |
| ATOM | 223 | HG1 | VAL | 120 | 2.722 | 10.090 | 0.735 | 1.00 | 0.00 | 0.053 | 0.00 | 0.00 |
| ATOM | 224 | HG1 | VAL | 120 | 1.766 | 8.607 | 0.964 | 1.00 | 0.00 | 0.053 | 0.00 | 0.00 |
| ATOM | 225 | CG2 | VAL | 120 | 0.596 | 9.324 | 3.360 | 1.00 | 0.00 | −0.159 | 16.15 | 4.00 |
| ATOM | 226 | HG2 | VAL | 120 | −0.276 | 9.825 | 2.940 | 1.00 | 0.00 | 0.053 | 0.00 | 0.00 |
| ATOM | 227 | HG2 | VAL | 120 | 0.523 | 8.252 | 3.171 | 1.00 | 0.00 | 0.053 | 0.00 | 0.00 |
| ATOM | 228 | HG2 | VAL | 120 | 0.634 | 9.501 | 4.435 | 1.00 | 0.00 | 0.053 | 0.00 | 0.00 |
| ATOM | 229 | C | VAL | 120 | 3.117 | 7.657 | 2.937 | 1.00 | 0.00 | 0.396 | 9.82 | 4.00 |
| ATOM | 230 | O | VAL | 120 | 2.421 | 6.852 | 3.557 | 1.00 | 0.00 | −0.396 | 8.17 | −17.40 |
| ATOM | 231 | N | ARG | 121G | 3.929 | 7.294 | 1.935 | 1.00 | 0.00 | −0.650 | 9.00 | −17.40 |
| ATOM | 232 | HN | ARG | 121G | 4.469 | 8.023 | 1.448 | 1.00 | 0.00 | 0.440 | 0.00 | 0.00 |
| ATOM | 233 | CA | ARG | 121G | 4.080 | 5.898 | 1.503 | 1.00 | 0.00 | 0.158 | 9.40 | 4.00 |
| ATOM | 234 | HA | ARG | 121G | 3.131 | 5.530 | 1.113 | 1.00 | 0.00 | 0.053 | 0.00 | 0.00 |
| ATOM | 235 | CB | ARG | 121G | 5.142 | 5.783 | 0.382 | 1.00 | 0.00 | −0.106 | 12.77 | 4.00 |
| ATOM | 236 | HB1 | ARG | 121G | 6.087 | 6.162 | 0.768 | 1.00 | 0.00 | 0.053 | 0.00 | 0.00 |
| ATOM | 237 | HB2 | ARG | 121G | 4.806 | 6.377 | −0.467 | 1.00 | 0.00 | 0.053 | 0.00 | 0.00 |
| ATOM | 238 | CG | ARG | 121G | 5.389 | 4.348 | −0.123 | 1.00 | 0.00 | −0.106 | 12.77 | 4.00 |
| ATOM | 239 | HG1 | ARG | 121G | 4.502 | 3.908 | −0.580 | 1.00 | 0.00 | 0.053 | 0.00 | 0.00 |
| ATOM | 240 | HG2 | ARG | 121G | 5.689 | 3.669 | 0.674 | 1.00 | 0.00 | 0.053 | 0.00 | 0.00 |
| ATOM | 241 | CD | ARG | 121G | 6.506 | 4.281 | −1.192 | 1.00 | 0.00 | 0.374 | 12.77 | 4.00 |
| ATOM | 242 | HD1 | ARG | 121G | 6.758 | 3.261 | −1.484 | 1.00 | 0.00 | 0.053 | 0.00 | 0.00 |
| ATOM | 243 | HD2 | ARG | 121G | 7.441 | 4.731 | −0.862 | 1.00 | 0.00 | 0.053 | 0.00 | 0.00 |
| ATOM | 244 | NE | ARG | 121G | 6.155 | 4.968 | −2.430 | 1.00 | 0.00 | −0.819 | 9.00 | −24.67 |
| ATOM | 245 | HE | ARG | 121G | 6.554 | 5.904 | −2.586 | 1.00 | 0.00 | 0.407 | 0.00 | 0.00 |
| ATOM | 246 | CZ | ARG | 121G | 5.358 | 4.474 | −3.371 | 1.00 | 0.00 | 0.796 | 6.95 | 4.00 |
| ATOM | 247 | NH1 | ARG | 121G | 4.818 | 3.265 | −3.225 | 1.00 | 0.00 | −0.746 | 9.00 | −24.67 |
| ATOM | 248 | HH1 | ARG | 121G | 4.200 | 2.885 | −3.957 | 1.00 | 0.00 | 0.407 | 0.00 | 0.00 |
| ATOM | 249 | HH1 | ARG | 121G | 5.018 | 2.710 | −2.381 | 1.00 | 0.00 | 0.407 | 0.00 | 0.00 |
| ATOM | 250 | NH2 | ARG | 121G | 5.101 | 5.191 | −4.463 | 1.00 | 0.00 | −0.746 | 9.00 | −24.67 |
| ATOM | 251 | HH2 | ARG | 121G | 4.483 | 4.811 | −5.195 | 1.00 | 0.00 | 0.407 | 0.00 | 0.00 |
| ATOM | 252 | HH2 | ARG | 121G | 5.519 | 6.125 | −4.576 | 1.00 | 0.00 | 0.407 | 0.00 | 0.00 |
| ATOM | 253 | C | ARG | 121G | 4.507 | 5.033 | 2.694 | 1.00 | 0.00 | 0.396 | 9.82 | 4.00 |
| ATOM | 254 | O | ARG | 121G | 3.917 | 3.990 | 2.969 | 1.00 | 0.00 | −0.396 | 8.17 | −17.40 |
| ATOM | 255 | N | ARG | 122G | 5.532 | 5.476 | 3.410 | 1.00 | 0.00 | −0.650 | 9.00 | −17.40 |
| ATOM | 256 | HN | ARG | 122G | 5.989 | 6.360 | 3.148 | 1.00 | 0.00 | 0.440 | 0.00 | 0.00 |
| ATOM | 257 | CA | ARG | 122G | 6.017 | 4.732 | 4.557 | 1.00 | 0.00 | 0.158 | 9.40 | 4.00 |
| ATOM | 258 | HA | ARG | 122G | 6.354 | 3.739 | 4.258 | 1.00 | 0.00 | 0.053 | 0.00 | 0.00 |
| ATOM | 259 | CB | ARG | 122G | 7.234 | 5.443 | 5.156 | 1.00 | 0.00 | −0.106 | 12.77 | 4.00 |
| ATOM | 260 | HB1 | ARG | 122G | 6.938 | 6.463 | 5.402 | 1.00 | 0.00 | 0.053 | 0.00 | 0.00 |
| ATOM | 261 | HB2 | ARG | 122G | 8.027 | 5.435 | 4.409 | 1.00 | 0.00 | 0.053 | 0.00 | 0.00 |
| ATOM | 262 | CG | ARG | 122G | 7.804 | 4.827 | 6.418 | 1.00 | 0.00 | −0.106 | 12.77 | 4.00 |
| ATOM | 263 | HG1 | ARG | 122G | 8.079 | 3.788 | 6.233 | 1.00 | 0.00 | 0.053 | 0.00 | 0.00 |
| ATOM | 264 | HG2 | ARG | 122G | 7.062 | 4.860 | 7.216 | 1.00 | 0.00 | 0.053 | 0.00 | 0.00 |
| ATOM | 265 | CD | ARG | 122G | 9.047 | 5.601 | 6.861 | 1.00 | 0.00 | 0.374 | 12.77 | 4.00 |
| ATOM | 266 | HD1 | ARG | 122G | 8.840 | 6.670 | 6.912 | 1.00 | 0.00 | 0.053 | 0.00 | 0.00 |
| ATOM | 267 | HD2 | ARG | 122G | 9.868 | 5.448 | 6.161 | 1.00 | 0.00 | 0.053 | 0.00 | 0.00 |
| ATOM | 268 | NE | ARG | 122G | 9.490 | 5.161 | 8.184 | 1.00 | 0.00 | −0.819 | 9.00 | −24.67 |
| ATOM | 269 | HE | ARG | 122G | 8.889 | 4.495 | 8.692 | 1.00 | 0.00 | 0.407 | 0.00 | 0.00 |
| ATOM | 270 | CZ | ARG | 122G | 10.614 | 5.558 | 8.780 | 1.00 | 0.00 | 0.796 | 6.95 | 4.00 |
| ATOM | 271 | NH1 | ARG | 122G | 11.429 | 6.408 | 8.175 | 1.00 | 0.00 | −0.746 | 9.00 | −24.67 |
| ATOM | 272 | HH1 | ARG | 122G | 12.296 | 6.710 | 8.641 | 1.00 | 0.00 | 0.407 | 0.00 | 0.00 |
| ATOM | 273 | HH1 | ARG | 122G | 11.196 | 6.766 | 7.238 | 1.00 | 0.00 | 0.407 | 0.00 | 0.00 |
| ATOM | 274 | NH2 | ARG | 122G | 10.914 | 5.111 | 9.994 | 1.00 | 0.00 | −0.746 | 9.00 | −24.67 |
| ATOM | 275 | HH2 | ARG | 122G | 11.782 | 5.417 | 10.455 | 1.00 | 0.00 | 0.407 | 0.00 | 0.00 |
| ATOM | 276 | HH2 | ARG | 122G | 10.278 | 4.458 | 10.474 | 1.00 | 0.00 | 0.407 | 0.00 | 0.00 |
| ATOM | 277 | C | ARG | 122G | 4.920 | 4.568 | 5.594 | 1.00 | 0.00 | 0.396 | 9.82 | 4.00 |
| ATOM | 278 | O | ARG | 122G | 4.780 | 3.495 | 6.191 | 1.00 | 0.00 | −0.396 | 8.17 | −17.40 |
| ATOM | 279 | N | CYSH | 123S | 4.141 | 5.628 | 5.811 | 1.00 | 0.00 | −0.65 | 9.00 | −17.40 |
| ATOM | 280 | HN | CYSH | 123S | 4.313 | 6.497 | 5.286 | 1.00 | 0.00 | 0.44 | 0.00 | 0.00 |
| ATOM | 281 | CA | CYSH | 123S | 3.053 | 5.582 | 6.773 | 1.00 | 0.00 | 0.15 | 9.40 | 4.00 |
| ATOM | 282 | HA | CYSH | 123S | 3.446 | 5.330 | 7.758 | 1.00 | 0.00 | 0.05 | 0.00 | 0.00 |
| ATOM | 283 | CB | CYSH | 123S | 2.374 | 6.958 | 6.867 | 1.00 | 0.00 | −0.04 | 12.77 | 4.00 |
| ATOM | 284 | HB1 | CYSH | 123S | 2.053 | 7.247 | 5.866 | 1.00 | 0.00 | 0.05 | 0.00 | 0.00 |
| ATOM | 285 | HB2 | CYSH | 123S | 3.100 | 7.668 | 7.261 | 1.00 | 0.00 | 0.05 | 0.00 | 0.00 |
| ATOM | 286 | SG | CYSH | 123S | 0.913 | 7.019 | 7.945 | 1.00 | 0.00 | −0.25 | 19.93 | −6.40 |
| ATOM | 287 | HG | CYSH | 123S | 0.595 | 8.296 | 8.196 | 1.00 | 0.00 | 0.19 | 0.00 | 0.00 |
| ATOM | 288 | C | CYSH | 123S | 2.041 | 4.526 | 6.350 | 1.00 | 0.00 | 0.39 | 9.82 | 4.00 |
| ATOM | 289 | O | CYSH | 123S | 1.714 | 3.631 | 7.136 | 1.00 | 0.00 | −0.39 | 8.17 | −17.40 |
| ATOM | 290 | N | LEU | 124 | 1.573 | 4.609 | 5.102 | 1.00 | 0.00 | −0.650 | 9.00 | −17.40 |
| ATOM | 291 | HN | LEU | 124 | 1.922 | 5.348 | 4.475 | 1.00 | 0.00 | 0.440 | 0.00 | 0.00 |
| ATOM | 292 | CA | LEU | 124 | 0.575 | 3.667 | 4.616 | 1.00 | 0.00 | 0.158 | 9.40 | 4.00 |
| ATOM | 293 | HA | LEU | 124 | −0.315 | 3.628 | 5.243 | 1.00 | 0.00 | 0.053 | 0.00 | 0.00 |
| ATOM | 294 | CB | LEU | 124 | 0.013 | 4.109 | 3.245 | 1.00 | 0.00 | −0.106 | 12.77 | 4.00 |
| ATOM | 295 | HB1 | LEU | 124 | −0.630 | 3.307 | 2.883 | 1.00 | 0.00 | 0.053 | 0.00 | 0.00 |
| ATOM | 296 | HB2 | LEU | 124 | −0.862 | 4.269 | 2.581 | 1.00 | 0.00 | 0.053 | 0.00 | 0.00 |
| ATOM | 297 | CG | LEU | 124 | −0.836 | 5.409 | 3.190 | 1.00 | 0.00 | −0.053 | 9.40 | 4.00 |
| ATOM | 298 | HG | LEU | 124 | −0.242 | 6.257 | 3.529 | 1.00 | 0.00 | 0.053 | 0.00 | 0.00 |
| ATOM | 299 | CD1 | LEU | 124 | −1.300 | 5.672 | 1.758 | 1.00 | 0.00 | −0.159 | 16.15 | 4.00 |
| ATOM | 300 | HD1 | LEU | 124 | −1.894 | 6.585 | 1.730 | 1.00 | 0.00 | 0.053 | 0.00 | 0.00 |
| ATOM | 301 | HD1 | LEU | 124 | −0.431 | 5.784 | 1.109 | 1.00 | 0.00 | 0.053 | 0.00 | 0.00 |
| ATOM | 302 | HD1 | LEU | 124 | −1.905 | 4.834 | 1.412 | 1.00 | 0.00 | 0.053 | 0.00 | 0.00 |
| ATOM | 303 | CD2 | LEU | 124 | −2.062 | 5.280 | 4.106 | 1.00 | 0.00 | −0.159 | 16.15 | 4.00 |
| ATOM | 304 | HD2 | LEU | 124 | −2.649 | 6.197 | 4.059 | 1.00 | 0.00 | 0.053 | 0.00 | 0.00 |
| ATOM | 305 | HD2 | LEU | 124 | −2.674 | 4.439 | 3.778 | 1.00 | 0.00 | 0.053 | 0.00 | 0.00 |
| ATOM | 306 | HD2 | LEU | 124 | −1.733 | 5.111 | 5.131 | 1.00 | 0.00 | 0.053 | 0.00 | 0.00 |
| ATOM | 307 | C | LEU | 124 | 1.093 | 2.232 | 4.545 | 1.00 | 0.00 | 0.396 | 9.82 | 4.00 |
| ATOM | 308 | O | LEU | 124 | 0.364 | 1.306 | 4.891 | 1.00 | 0.00 | −0.396 | 8.17 | −17.40 |
| ATOM | 309 | N | ASP | 125P | 2.342 | 2.028 | 4.124 | 1.00 | 0.00 | −0.650 | 9.00 | −17.40 |
| ATOM | 310 | HN | ASP | 125P | 2.940 | 2.820 | 3.852 | 1.00 | 0.00 | 0.440 | 0.00 | 0.00 |
| ATOM | 311 | CA | ASP | 125P | 2.845 | 0.656 | 4.056 | 1.00 | 0.00 | 0.158 | 9.40 | 4.00 |
| ATOM | 312 | HA | ASP | 125P | 2.300 | 0.058 | 3.324 | 1.00 | 0.00 | 0.053 | 0.00 | 0.00 |
| ATOM | 313 | CB | ASP | 125P | 4.306 | 0.599 | 3.585 | 1.00 | 0.00 | −0.336 | 12.77 | 4.00 |
| ATOM | 314 | HB1 | ASP | 125P | 4.748 | −0.390 | 3.701 | 1.00 | 0.00 | 0.053 | 0.00 | 0.00 |
| ATOM | 315 | HB2 | ASP | 125P | 4.953 | 1.283 | 4.134 | 1.00 | 0.00 | 0.053 | 0.00 | 0.00 |
| ATOM | 316 | CG | ASP | 125P | 4.466 | 0.961 | 2.110 | 1.00 | 0.00 | 0.297 | 9.82 | 4.00 |
| ATOM | 317 | OD1 | ASP | 125P | 3.486 | 0.881 | 1.331 | 1.00 | 0.00 | −0.534 | 8.17 | −18.95 |
| ATOM | 318 | OD2 | ASP | 125P | 5.596 | 1.314 | 1.724 | 1.00 | 0.00 | −0.534 | 8.17 | −18.95 |
| ATOM | 319 | C | ASP | 125P | 2.722 | −0.064 | 5.397 | 1.00 | 0.00 | 0.396 | 9.82 | 4.00 |
| ATOM | 320 | O | ASP | 125P | 2.187 | −1.171 | 5.449 | 1.00 | 0.00 | −0.396 | 8.17 | −17.40 |
| ATOM | 321 | N | LYS | 126S | 3.175 | 0.578 | 6.474 | 1.00 | 0.00 | −0.650 | 9.00 | −17.40 |
| ATOM | 322 | HN | LYS | 126S | 3.568 | 1.524 | 6.368 | 1.00 | 0.00 | 0.440 | 0.00 | 0.00 |
| ATOM | 323 | CA | LYS | 126S | 3.125 | −0.035 | 7.806 | 1.00 | 0.00 | 0.158 | 9.40 | 4.00 |
| ATOM | 324 | HA | LYS | 126S | 3.619 | −1.005 | 7.754 | 1.00 | 0.00 | 0.053 | 0.00 | 0.00 |
| ATOM | 325 | CB | LYS | 126S | 3.918 | 0.808 | 8.802 | 1.00 | 0.00 | −0.106 | 12.77 | 4.00 |
| ATOM | 326 | HB1 | LYS | 126S | 3.717 | 0.430 | 9.804 | 1.00 | 0.00 | 0.053 | 0.00 | 0.00 |
| ATOM | 327 | HB2 | LYS | 126S | 3.591 | 1.843 | 8.708 | 1.00 | 0.00 | 0.053 | 0.00 | 0.00 |
| ATOM | 328 | CG | LYS | 126S | 5.425 | 0.781 | 8.594 | 1.00 | 0.00 | −0.106 | 12.77 | 4.00 |
| ATOM | 329 | HG1 | LYS | 126S | 5.915 | 1.455 | 9.296 | 1.00 | 0.00 | 0.053 | 0.00 | 0.00 |
| ATOM | 330 | HG2 | LYS | 126S | 5.671 | 1.093 | 7.579 | 1.00 | 0.00 | 0.053 | 0.00 | 0.00 |
| ATOM | 331 | CD | LYS | 126S | 5.965 | −0.632 | 8.815 | 1.00 | 0.00 | 0.106 | 12.77 | 4.00 |
| ATOM | 332 | HD1 | LYS | 126S | 5.570 | −1.337 | 8.083 | 1.00 | 0.00 | 0.053 | 0.00 | 0.00 |
| ATOM | 333 | HD2 | LYS | 126S | 5.704 | −1.017 | 9.800 | 1.00 | 0.00 | 0.053 | 0.00 | 0.00 |
| ATOM | 334 | CE | LYS | 126S | 7.486 | −0.684 | 8.706 | 1.00 | 0.00 | 0.099 | 12.77 | 4.00 |
| ATOM | 335 | HE1 | LYS | 126S | 7.899 | −1.672 | 8.907 | 1.00 | 0.00 | 0.053 | 0.00 | 0.00 |
| ATOM | 336 | HE2 | LYS | 126S | 7.991 | −0.012 | 9.400 | 1.00 | 0.00 | 0.053 | 0.00 | 0.00 |
| ATOM | 337 | NZ | LYS | 126S | 7.996 | −0.310 | 7.351 | 1.00 | 0.00 | −0.045 | 13.25 | −39.20 |
| ATOM | 338 | HZ1 | LYS | 126S | 9.024 | −0.364 | 7.343 | 1.00 | 0.00 | 0.280 | 0.00 | 0.00 |
| ATOM | 339 | HZ2 | LYS | 126S | 7.613 | −0.958 | 6.648 | 1.00 | 0.00 | 0.280 | 0.00 | 0.00 |
| ATOM | 340 | HZ3 | LYS | 126S | 7.702 | 0.651 | 7.126 | 1.00 | 0.00 | 0.280 | 0.00 | 0.00 |
| ATOM | 341 | C | LYS | 126S | 1.713 | −0.265 | 8.347 | 1.00 | 0.00 | 0.396 | 9.82 | 4.00 |
| ATOM | 342 | O | LYS | 126S | 1.438 | −1.311 | 8.938 | 1.00 | 0.00 | −0.396 | 8.17 | −17.40 |
| ATOM | 343 | N | LEU | 127 | 0.817 | 0.703 | 8.150 | 1.00 | 0.00 | −0.650 | 9.00 | −17.40 |
| ATOM | 344 | HN | LEU | 127 | 1.099 | 1.562 | 7.657 | 1.00 | 0.00 | 0.440 | 0.00 | 0.00 |
| ATOM | 345 | CA | LEU | 127 | −0.562 | 0.564 | 8.622 | 1.00 | 0.00 | 0.158 | 9.40 | 4.00 |
| ATOM | 346 | HA | LEU | 127 | −0.576 | 0.405 | 9.700 | 1.00 | 0.00 | 0.053 | 0.00 | 0.00 |
| ATOM | 347 | CB | LEU | 127 | −1.382 | 1.816 | 8.290 | 1.00 | 0.00 | −0.106 | 12.77 | 4.00 |
| ATOM | 348 | HB1 | LEU | 127 | −2.432 | 1.527 | 8.330 | 1.00 | 0.00 | 0.053 | 0.00 | 0.00 |
| ATOM | 349 | HB2 | LEU | 127 | −1.085 | 2.133 | 7.290 | 1.00 | 0.00 | 0.053 | 0.00 | 0.00 |
| ATOM | 350 | CG | LEU | 127 | −1.261 | 3.054 | 9.168 | 1.00 | 0.00 | −0.053 | 9.40 | 4.00 |
| ATOM | 351 | HG | LEU | 127 | −0.208 | 3.266 | 9.356 | 1.00 | 0.00 | 0.053 | 0.00 | 0.00 |
| ATOM | 352 | CD1 | LEU | 127 | −1.904 | 4.254 | 8.457 | 1.00 | 0.00 | −0.159 | 16.15 | 4.00 |
| ATOM | 353 | HD1 | LEU | 127 | −1.816 | 5.139 | 9.086 | 1.00 | 0.00 | 0.053 | 0.00 | 0.00 |
| ATOM | 354 | HD1 | LEU | 127 | −1.395 | 4.430 | 7.509 | 1.00 | 0.00 | 0.053 | 0.00 | 0.00 |
| ATOM | 355 | HD1 | LEU | 127 | −2.957 | 4.044 | 8.269 | 1.00 | 0.00 | 0.053 | 0.00 | 0.00 |
| ATOM | 356 | CD2 | LEU | 127 | −1.950 | 2.806 | 10.483 | 1.00 | 0.00 | −0.159 | 16.15 | 4.00 |
| ATOM | 357 | HD2 | LEU | 127 | −1.863 | 3.692 | 11.112 | 1.00 | 0.00 | 0.053 | 0.00 | 0.00 |
| ATOM | 358 | HD2 | LEU | 127 | −3.003 | 2.587 | 10.306 | 1.00 | 0.00 | 0.053 | 0.00 | 0.00 |
| ATOM | 359 | HD2 | LEU | 127 | −1.482 | 1.958 | 10.984 | 1.00 | 0.00 | 0.053 | 0.00 | 0.00 |
| ATOM | 360 | C | LEU | 127 | −1.230 | −0.620 | 7.946 | 1.00 | 0.00 | 0.396 | 9.82 | 4.00 |
| ATOM | 361 | O | LEU | 127 | −1.886 | −1.433 | 8.583 | 1.00 | 0.00 | −0.396 | 8.17 | −17.40 |
| ATOM | 362 | N | LEU | 128 | −1.076 | −0.678 | 6.631 | 1.00 | 0.00 | −0.650 | 9.00 | −17.40 |
| ATOM | 363 | HN | LEU | 128 | −0.515 | 0.048 | 6.164 | 1.00 | 0.00 | 0.440 | 0.00 | 0.00 |
| ATOM | 364 | CA | LEU | 128 | −1.668 | −1.726 | 5.828 | 1.00 | 0.00 | 0.158 | 9.40 | 4.00 |
| ATOM | 365 | HA | LEU | 128 | −2.732 | −1.823 | 6.040 | 1.00 | 0.00 | 0.053 | 0.00 | 0.00 |
| ATOM | 366 | CB | LEU | 128 | −1.530 | −1.363 | 4.340 | 1.00 | 0.00 | −0.106 | 12.77 | 4.00 |
| ATOM | 367 | HB1 | LEU | 128 | −1.697 | −2.265 | 3.752 | 1.00 | 0.00 | 0.053 | 0.00 | 0.00 |
| ATOM | 368 | HB2 | LEU | 128 | −0.523 | −0.977 | 4.175 | 1.00 | 0.00 | 0.053 | 0.00 | 0.00 |
| ATOM | 369 | CG | LEU | 128 | −2.513 | −0.306 | 3.848 | 1.00 | 0.00 | −0.053 | 9.40 | 4.00 |
| ATOM | 370 | HG | LEU | 128 | −2.493 | 0.571 | 4.494 | 1.00 | 0.00 | 0.053 | 0.00 | 0.00 |
| ATOM | 371 | CD1 | LEU | 128 | −2.157 | 0.149 | 2.405 | 1.00 | 0.00 | −0.159 | 16.15 | 4.00 |
| ATOM | 372 | HD1 | LEU | 128 | −2.871 | 0.903 | 2.074 | 1.00 | 0.00 | 0.053 | 0.00 | 0.00 |
| ATOM | 373 | HD1 | LEU | 128 | −1.152 | 0.571 | 2.394 | 1.00 | 0.00 | 0.053 | 0.00 | 0.00 |
| ATOM | 374 | HD1 | LEU | 128 | −2.197 | −0.708 | 1.733 | 1.00 | 0.00 | 0.053 | 0.00 | 0.00 |
| ATOM | 375 | CD2 | LEU | 128 | −3.914 | −0.930 | 3.87S | 1.00 | 0.00 | −0.159 | 16.15 | 4.00 |
| ATOM | 376 | HD2 | LEU | 128 | −4.644 | −0.199 | 3.528 | 1.00 | 0.00 | 0.053 | 0.00 | 0.00 |
| ATOM | 377 | HD2 | LEU | 128 | −3.936 | −1.803 | 3.222 | 1.00 | 0.00 | 0.053 | 0.00 | 0.00 |
| ATOM | 378 | HD2 | LEU | 128 | −4.158 | −1.231 | 4.893 | 1.00 | 0.00 | 0.053 | 0.00 | 0.00 |
| ATOM | 379 | C | LEU | 128 | −1.009 | −3.061 | 6.112 | 1.00 | 0.00 | 0.396 | 9.82 | 4.00 |
| ATOM | 380 | O | LEU | 128 | −1.651 | −4.103 | 6.040 | 1.00 | 0.00 | −0.396 | 8.17 | −17.40 |
| ATOM | 381 | N | HIS | 129S | 0.279 | −3.032 | 6.438 | 1.00 | 0.00 | −0.650 | 9.00 | −17.40 |
| ATOM | 382 | HN | HIS | 129S | 0.775 | −2.131 | 6.488 | 1.00 | 0.00 | 0.440 | 0.00 | 0.00 |
| ATOM | 383 | CA | HIS | 129S | 0.989 | −4.268 | 6.724 | 1.00 | 0.00 | 0.158 | 9.40 | 4.00 |
| ATOM | 384 | HA | HIS | 129S | 0.843 | −4.998 | 5.927 | 1.00 | 0.00 | 0.053 | 0.00 | 0.00 |
| ATOM | 385 | CB | HIS | 129S | 2.490 | −4.008 | 6.821 | 1.00 | 0.00 | −0.106 | 12.77 | 4.00 |
| ATOM | 386 | HB1 | HIS | 129S | 2.757 | −3.280 | 7.587 | 1.00 | 0.00 | 0.053 | 0.00 | 0.00 |
| ATOM | 387 | HB2 | HIS | 129S | 2.926 | −3.622 | 5.899 | 1.00 | 0.00 | 0.053 | 0.00 | 0.00 |
| ATOM | 388 | CG | HIS | 129S | 3.290 | −5.231 | 7.149 | 1.00 | 0.00 | −0.050 | 7.26 | 0.60 |
| ATOM | 389 | CD2 | HIS | 129S | 3.763 | −5.694 | 8.332 | 1.00 | 0.00 | −0.177 | 10.80 | 0.60 |
| ATOM | 390 | HD2 | HIS | 129S | 3.681 | −5.199 | 9.300 | 1.00 | 0.00 | 0.127 | 0.00 | 0.00 |
| ATOM | 391 | ND1 | HIS | 129S | 3.626 | −6.178 | 6.205 | 1.00 | 0.00 | 0.207 | 9.25 | −17.40 |
| ATOM | 392 | HD1 | HIS | 129S | 3.409 | −6.120 | 5.199 | 1.00 | 0.00 | 0.393 | 0.00 | 0.00 |
| ATOM | 393 | CE1 | HIS | 129S | 4.270 | −7.172 | 6.792 | 1.00 | 0.00 | −0.227 | 10.80 | 0.60 |
| ATOM | 394 | HE1 | HIS | 129S | 4.657 | −8.062 | 6.295 | 1.00 | 0.00 | 0.127 | 0.00 | 0.00 |
| ATOM | 395 | NE2 | HIS | 129S | 4.365 | −6.904 | 8.082 | 1.00 | 0.00 | 0.207 | 9.25 | −17.40 |
| ATOM | 396 | HE2 | HIS | 129S | 4.817 | −7.504 | 8.786 | 1.00 | 0.00 | 0.393 | 0.00 | 0.00 |
| ATOM | 397 | C | HIS | 129S | 0.499 | −4.895 | 8.026 | 1.00 | 0.00 | 0.396 | 9.82 | 4.00 |
| ATOM | 398 | O | HIS | 129S | 0.314 | −6.103 | 8.113 | 1.00 | 0.00 | −0.396 | 8.17 | −17.40 |
| ATOM | 399 | N | TYR | 130 | 0.274 | −4.058 | 9.030 | 1.00 | 0.00 | −0.650 | 9.00 | −17.40 |
| ATOM | 400 | HN | TYR | 130 | 0.410 | −3.048 | 8.881 | 1.00 | 0.00 | 0.440 | 0.00 | 0.00 |
| ATOM | 401 | CA | TYR | 130 | −0.160 | −4.528 | 10.335 | 1.00 | 0.00 | 0.158 | 9.40 | 4.00 |
| ATOM | 402 | HA | TYR | 130 | 0.261 | −5.521 | 10.489 | 1.00 | 0.00 | 0.053 | 0.00 | 0.00 |
| ATOM | 403 | CB | TYR | 130 | 0.428 | −3.609 | 11.409 | 1.00 | 0.00 | −0.106 | 12.77 | 4.00 |
| ATOM | 404 | HB1 | TYR | 130 | 0.014 | −3.798 | 12.399 | 1.00 | 0.00 | 0.053 | 0.00 | 0.00 |
| ATOM | 405 | HB2 | TYR | 130 | 0.247 | −2.553 | 11.206 | 1.00 | 0.00 | 0.053 | 0.00 | 0.00 |
| ATOM | 406 | CG | TYR | 130 | 1.925 | −3.755 | 11.549 | 1.00 | 0.00 | 0.000 | 7.26 | 0.60 |
| ATOM | 407 | CD1 | TYR | 130 | 2.754 | −2.635 | 11.637 | 1.00 | 0.00 | −0.127 | 10.80 | 0.60 |
| ATOM | 408 | HD1 | TYR | 130 | 2.313 | −1.638 | 11.616 | 1.00 | 0.00 | 0.127 | 0.00 | 0.00 |
| ATOM | 409 | CE1 | TYR | 130 | 4.137 | −2.772 | 11.751 | 1.00 | 0.00 | −0.127 | 10.80 | 0.60 |
| ATOM | 410 | HE1 | TYR | 130 | 4.773 | −1.889 | 11.814 | 1.00 | 0.00 | 0.127 | 0.00 | 0.00 |
| ATOM | 411 | CD2 | TYR | 130 | 2.512 | −5.017 | 11.582 | 1.00 | 0.00 | −0.127 | 10.80 | 0.60 |
| ATOM | 412 | HD2 | TYR | 130 | 1.880 | −5.903 | 11.516 | 1.00 | 0.00 | 0.127 | 0.00 | 0.00 |
| ATOM | 413 | CE2 | TYR | 130 | 3.888 | −5.166 | 11.697 | 1.00 | 0.00 | −0.127 | 10.80 | 0.60 |
| ATOM | 414 | HE2 | TYR | 130 | 4.330 | −6.161 | 11.719 | 1.00 | 0.00 | 0.127 | 0.00 | 0.00 |
| ATOM | 415 | CZ | TYR | 130 | 4.696 | −4.042 | 11.783 | 1.00 | 0.00 | 0.026 | 7.26 | 0.60 |
| ATOM | 416 | OH | TYR | 130 | 6.063 | −4.202 | 11.934 | 1.00 | 0.00 | −0.451 | 10.94 | −17.40 |
| ATOM | 417 | HH | TYR | 130 | 6.290 | −4.291 | 12.934 | 1.00 | 0.00 | 0.424 | 0.00 | 0.00 |
| ATOM | 418 | C | TYR | 130 | −1.668 | −4.647 | 10.541 | 1.00 | 0.00 | 0.396 | 9.82 | 4.00 |
| ATOM | 419 | O | TYR | 130 | −2.106 | −5.329 | 11.453 | 1.00 | 0.00 | −0.396 | 8.17 | −17.40 |
| ATOM | 420 | N | ARG | 131G | −2.456 | −3.981 | 9.710 | 1.00 | 0.00 | −0.650 | 9.00 | −17.40 |
| ATOM | 421 | HN | ARG | 131G | −2.035 | −3.433 | 8.946 | 1.00 | 0.00 | 0.440 | 0.00 | 0.00 |
| ATOM | 422 | CA | ARG | 131G | −3.913 | −4.008 | 9.856 | 1.00 | 0.00 | 0.158 | 9.40 | 4.00 |
| ATOM | 423 | HA | ARG | 131G | −4.240 | −3.479 | 10.751 | 1.00 | 0.00 | 0.053 | 0.00 | 0.00 |
| ATOM | 424 | CB | ARG | 131G | −4.565 | −3.274 | 8.670 | 1.00 | 0.00 | −0.106 | 12.77 | 4.00 |
| ATOM | 425 | HB1 | ARG | 131G | −4.361 | −3.759 | 7.715 | 1.00 | 0.00 | 0.053 | 0.00 | 0.00 |
| ATOM | 426 | HB2 | ARG | 131G | −4.214 | −2.246 | 8.570 | 1.00 | 0.00 | 0.053 | 0.00 | 0.00 |
| ATOM | 427 | CG | ARG | 131G | −6.069 | −3.197 | 8.793 | 1.00 | 0.00 | −0.106 | 12.77 | 4.00 |
| ATOM | 428 | HG1 | ARG | 131G | −6.406 | −2.929 | 9.794 | 1.00 | 0.00 | 0.053 | 0.00 | 0.00 |
| ATOM | 429 | HG2 | ARG | 131G | −6.568 | −4.137 | 8.560 | 1.00 | 0.00 | 0.053 | 0.00 | 0.00 |
| ATOM | 430 | CD | ARG | 131G | −6.696 | −2.166 | 7.866 | 1.00 | 0.00 | 0.374 | 12.77 | 4.00 |
| ATOM | 431 | HD1 | ARG | 131G | −6.267 | −1.170 | 7.986 | 1.00 | 0.00 | 0.053 | 0.00 | 0.00 |
| ATOM | 432 | HD2 | ARG | 131G | −7.768 | −2.048 | 8.024 | 1.00 | 0.00 | 0.053 | 0.00 | 0.00 |
| ATOM | 433 | NE | ARG | 131G | −6.537 | −2.499 | 6.455 | 1.00 | 0.00 | −0.819 | 9.00 | −24.67 |
| ATOM | 434 | HE | ARG | 131G | −5.853 | −3.225 | 6.196 | 1.00 | 0.00 | 0.407 | 0.00 | 0.00 |
| ATOM | 435 | CZ | ARG | 131G | −7.231 | −1.911 | 5.487 | 1.00 | 0.00 | 0.796 | 6.95 | 4.00 |
| ATOM | 436 | NH1 | ARG | 131G | −8.125 | −0.979 | 5.800 | 1.00 | 0.00 | −0.746 | 9.00 | −24.67 |
| ATOM | 437 | HH1 | ARG | 131G | −8.668 | −0.516 | 5.057 | 1.00 | 0.00 | 0.407 | 0.00 | 0.00 |
| ATOM | 438 | HH1 | ARG | 131G | −8.274 | −0.719 | 6.785 | 1.00 | 0.00 | 0.407 | 0.00 | 0.00 |
| ATOM | 439 | NH2 | ARG | 131G | −7.040 | −2.253 | 4.218 | 1.00 | 0.00 | −0.746 | 9.00 | −24.67 |
| ATOM | 440 | HH2 | ARG | 131G | −7.582 | −1.792 | 3.473 | 1.00 | 0.00 | 0.407 | 0.00 | 0.00 |
| ATOM | 441 | HH2 | ARG | 131G | −6.349 | −2.978 | 3.978 | 1.00 | 0.00 | 0.407 | 0.00 | 0.00 |
| ATOM | 442 | C | ARG | 131G | −4.449 | −5.442 | 9.960 | 1.00 | 0.00 | 0.396 | 9.82 | 4.00 |
| ATOM | 443 | O | ARG | 131G | −4.204 | −6.262 | 9.084 | 1.00 | 0.00 | −0.396 | 8.17 | −17.40 |
| ATOM | 444 | N | PRO | 132 | −5.162 | −5.774 | 11.054 | 1.00 | 0.00 | −0.422 | 9.00 | −17.40 |
| ATOM | 445 | CD | PRO | 132 | −5.111 | −5.140 | 12.382 | 1.00 | 0.00 | 0.105 | 12.77 | 4.00 |
| ATOM | 446 | HD1 | PRO | 132 | −5.405 | −4.091 | 12.332 | 1.00 | 0.00 | 0.053 | 0.00 | 0.00 |
| ATOM | 447 | HD2 | PRO | 132 | −4.105 | −5.180 | 12.801 | 1.00 | 0.00 | 0.053 | 0.00 | 0.00 |
| ATOM | 448 | CA | PRO | 132 | −5.679 | −7.150 | 11.160 | 1.00 | 0.00 | 0.158 | 9.40 | 4.00 |
| ATOM | 449 | HA | PRO | 132 | −4.945 | −7.820 | 10.713 | 1.00 | 0.00 | 0.053 | 0.00 | 0.00 |
| ATOM | 450 | CB | PRO | 132 | −5.780 | −7.361 | 12.670 | 1.00 | 0.00 | −0.106 | 12.77 | 4.00 |
| ATOM | 451 | HB1 | PRO | 132 | −4.843 | −7.740 | 13.078 | 1.00 | 0.00 | 0.053 | 0.00 | 0.00 |
| ATOM | 452 | HB2 | PRO | 132 | −6.563 | −8.077 | 12.916 | 1.00 | 0.00 | 0.053 | 0.00 | 0.00 |
| ATOM | 453 | CG | PRO | 132 | −6.099 | −5.976 | 13.177 | 1.00 | 0.00 | −0.106 | 12.77 | 4.00 |
| ATOM | 454 | HG1 | PRO | 132 | −5.945 | −5.897 | 14.253 | 1.00 | 0.00 | 0.053 | 0.00 | 0.00 |
| ATOM | 455 | HG2 | PRO | 132 | −7.135 | −5.704 | 12.977 | 1.00 | 0.00 | 0.053 | 0.00 | 0.00 |
| ATOM | 456 | C | PRO | 132 | −7.012 | −7.409 | 10.461 | 1.00 | 0.00 | 0.396 | 9.82 | 4.00 |
| ATOM | 457 | O | PRO | 132 | −7.433 | −8.553 | 10.307 | 1.00 | 0.00 | −0.396 | 8.17 | −17.40 |
| ATOM | 458 | N | SER | 133 | −7.676 | −6.338 | 10.041 | 1.00 | 0.00 | −0.650 | 9.00 | −17.40 |
| ATOM | 459 | HN | SER | 133 | −7.263 | −5.404 | 10.179 | 1.00 | 0.00 | 0.440 | 0.00 | 0.00 |
| ATOM | 460 | CA | SER | 133 | −8.967 | −6.453 | 9.393 | 1.00 | 0.00 | 0.158 | 9.40 | 4.00 |
| ATOM | 461 | HA | SER | 133 | −8.961 | −7.269 | 8.671 | 1.00 | 0.00 | 0.053 | 0.00 | 0.00 |
| ATOM | 462 | CB | SER | 133 | −10.045 | −6.720 | 10.441 | 1.00 | 0.00 | 0.007 | 12.77 | 4.00 |
| ATOM | 463 | HB1 | SER | 133 | −9.905 | −6.092 | 11.321 | 1.00 | 0.00 | 0.053 | 0.00 | 0.00 |
| ATOM | 464 | HB2 | SER | 133 | −10.027 | −7.758 | 10.771 | 1.00 | 0.00 | 0.053 | 0.00 | 0.00 |
| ATOM | 465 | OG | SER | 133 | −11.342 | −6.455 | 9.926 | 1.00 | 0.00 | −0.537 | 11.04 | −17.40 |
| ATOM | 466 | HG | SER | 133 | −11.474 | −5.437 | 9.835 | 1.00 | 0.00 | 0.424 | 0.00 | 0.00 |
| ATOM | 467 | C | SER | 133 | −9.309 | −5.178 | 8.665 | 1.00 | 0.00 | 0.396 | 9.82 | 4.00 |
| ATOM | 468 | O | SER | 133 | −9.390 | −4.121 | 9.291 | 1.00 | 0.00 | −0.396 | 8.17 | −17.40 |
| ATOM | 469 | N | ALA | 134 | −9.540 | −5.286 | 7.356 | 1.00 | 0.00 | −0.650 | 9.00 | −17.40 |
| ATOM | 470 | HN | ALA | 134 | −9.464 | −6.209 | 6.905 | 1.00 | 0.00 | 0.440 | 0.00 | 0.00 |
| ATOM | 471 | CA | ALA | 134 | −9.895 | −4.130 | 6.554 | 1.00 | 0.00 | 0.158 | 9.40 | 4.00 |
| ATOM | 472 | HA | ALA | 134 | −9.159 | −3.337 | 6.691 | 1.00 | 0.00 | 0.053 | 0.00 | 0.00 |
| ATOM | 473 | CB | ALA | 134 | −9.911 | −4.507 | 5.077 | 1.00 | 0.00 | −0.159 | 16.15 | 4.00 |
| ATOM | 474 | HB1 | ALA | 134 | −10.178 | −3.634 | 4.481 | 1.00 | 0.00 | 0.053 | 0.00 | 0.00 |
| ATOM | 475 | HB2 | ALA | 134 | −8.923 | −4.859 | 4.780 | 1.00 | 0.00 | 0.053 | 0.00 | 0.00 |
| ATOM | 476 | HB3 | ALA | 134 | −10.643 | −5.297 | 4.911 | 1.00 | 0.00 | 0.053 | 0.00 | 0.00 |
| ATOM | 477 | C | ALA | 134 | −11.265 | −3.605 | 6.967 | 1.00 | 0.00 | 0.396 | 9.82 | 4.00 |
| ATOM | 478 | O | ALA | 134 | −11.490 | −2.396 | 6.998 | 1.00 | 0.00 | −0.396 | 8.17 | −17.40 |
| ATOM | 479 | N | GLU | 135 | −12.182 | −4.517 | 7.287 | 1.00 | 0.00 | −0.650 | 9.00 | −17.40 |
| ATOM | 480 | HN | GLU | 135 | −11.938 | −5.517 | 7.253 | 1.00 | 0.00 | 0.440 | 0.00 | 0.00 |
| ATOM | 481 | CA | GLU | 135 | −13.525 | −4.115 | 7.684 | 1.00 | 0.00 | 0.158 | 9.40 | 4.00 |
| ATOM | 482 | HA | GLU | 135 | −13.996 | −3.439 | 6.970 | 1.00 | 0.00 | 0.053 | 0.00 | 0.00 |
| ATOM | 483 | CB | GLU | 135 | −14.479 | −5.321 | 7.735 | 1.00 | 0.00 | −0.106 | 12.77 | 4.00 |
| ATOM | 484 | HB1 | GLU | 135 | −15.124 | −5.277 | 6.857 | 1.00 | 0.00 | 0.053 | 0.00 | 0.00 |
| ATOM | 485 | HB2 | GLU | 135 | −15.064 | −5.249 | 8.651 | 1.00 | 0.00 | 0.053 | 0.00 | 0.00 |
| ATOM | 486 | CG | GLU | 135 | −13.803 | −6.693 | 7.734 | 1.00 | 0.00 | −0.106 | 12.77 | 4.00 |
| ATOM | 487 | HG1 | GLU | 135 | −14.487 | −7.498 | 8.001 | 1.00 | 0.00 | 0.053 | 0.00 | 0.00 |
| ATOM | 488 | HG2 | GLU | 135 | −12.974 | −6.753 | 8.439 | 1.00 | 0.00 | 0.053 | 0.00 | 0.00 |
| ATOM | 489 | CD | GLU | 135 | −13.229 | −7.061 | 6.377 | 1.00 | 0.00 | 0.399 | 9.82 | 4.00 |
| ATOM | 490 | OE1 | GLU | 135 | −14.006 | −7.070 | 5.398 | 1.00 | 0.00 | −0.396 | 8.17 | −18.95 |
| ATOM | 491 | OE2 | GLU | 135 | −12.008 | −7.341 | 6.285 | 1.00 | 0.00 | −0.427 | 8.17 | −18.95 |
| ATOM | 492 | HE2 | GLU | 135 | −11.779 | −7.566 | 5.306 | 1.00 | 0.00 | 0.424 | 0.00 | 0.00 |
| ATOM | 493 | C | GLU | 135 | −13.558 | −3.390 | 9.021 | 1.00 | 0.00 | 0.396 | 9.82 | 4.00 |
| ATOM | 494 | O | GLU | 135 | −14.308 | −2.436 | 9.186 | 1.00 | 0.00 | −0.396 | 8.17 | −17.40 |
| ATOM | 495 | N | LEU | 136 | −12.747 | −3.836 | 9.974 | 1.00 | 0.00 | −0.650 | 9.00 | −17.40 |
| ATOM | 496 | HN | LEU | 136 | −12.131 | −4.640 | 9.785 | 1.00 | 0.00 | 0.440 | 0.00 | 0.00 |
| ATOM | 497 | CA | LEU | 136 | −12.720 | −3.198 | 11.284 | 1.00 | 0.00 | 0.158 | 9.40 | 4.00 |
| ATOM | 498 | HA | LEU | 136 | −13.714 | −2.976 | 11.671 | 1.00 | 0.00 | 0.053 | 0.00 | 0.00 |
| ATOM | 499 | CB | LEU | 136 | −12.092 | −4.133 | 12.316 | 1.00 | 0.00 | −0.106 | 12.77 | 4.00 |
| ATOM | 500 | HB1 | LEU | 136 | −11.976 | −3.666 | 13.294 | 1.00 | 0.00 | 0.053 | 0.00 | 0.00 |
| ATOM | 501 | HB2 | LEU | 136 | −11.097 | −4.473 | 12.028 | 1.00 | 0.00 | 0.053 | 0.00 | 0.00 |
| ATOM | 502 | CG | LEU | 136 | −12.920 | −5.403 | 12.550 | 1.00 | 0.00 | −0.053 | 9.40 | 4.00 |
| ATOM | 503 | HG | LEU | 136 | −12.902 | −6.021 | 11.652 | 1.00 | 0.00 | 0.053 | 0.00 | 0.00 |
| ATOM | 504 | CD1 | LEU | 136 | −12.332 | −6.188 | 13.716 | 1.00 | 0.00 | −0.159 | 16.15 | 4.00 |
| ATOM | 505 | HD1 | LEU | 136 | −12.921 | −7.090 | 13.881 | 1.00 | 0.00 | 0.053 | 0.00 | 0.00 |
| ATOM | 506 | HD1 | LEU | 136 | −11.302 | −6.463 | 13.486 | 1.00 | 0.00 | 0.053 | 0.00 | 0.00 |
| ATOM | 507 | HD1 | LEU | 136 | −12.350 | −5.572 | 14.615 | 1.00 | 0.00 | 0.053 | 0.00 | 0.00 |
| ATOM | 508 | CD2 | LEU | 136 | −14.377 | −5.019 | 12.849 | 1.00 | 0.00, | −0.159 | 16.15 | 4.00 |
| ATOM | 509 | HD2 | LEU | 136 | −14.964 | −5.922 | 13.015 | 1.00 | 0.00 | 0.053 | 0.00 | 0.00 |
| ATOM | 510 | HD2 | LEU | 136 | −14.412 | −4.393 | 13.741 | 1.00 | 0.00 | 0.053 | 0.00 | 0.00 |
| ATOM | 511 | HD2 | LEU | 136 | −14.789 | −4.468 | 12.003 | 1.00 | 0.00 | 0.053 | 0.00 | 0.00 |
| ATOM | 512 | C | LEU | 136 | −11.974 | −1.873 | 11.285 | 1.00 | 0.00 | 0.396 | 9.82 | 4.00 |
| ATOM | 513 | O | LEU | 136 | −12.360 | −0.937 | 12.003 | 1.00 | 0.00 | −0.396 | 8.17 | −17.40 |
| ATOM | 514 | N | PHE | 137 | −10.911 | −1.803 | 10.482 | 1.00 | 0.00 | −0.650 | 9.00 | −17.40 |
| ATOM | 515 | HN | PHE | 137 | −10.660 | −2.625 | 9.915 | 1.00 | 0.00 | 0.440 | 0.00 | 0.00 |
| ATOM | 516 | CA | PHE | 137 | −10.093 | −0.601 | 10.380 | 1.00 | 0.00 | 0.158 | 9.40 | 4.00 |
| ATOM | 517 | HA | PHE | 137 | −10.543 | 0.168 | 11.007 | 1.00 | 0.00 | 0.053 | 0.00 | 0.00 |
| ATOM | 518 | CB | PHE | 137 | −8.681 | −0.861 | 10.920 | 1.00 | 0.00 | −0.106 | 12.77 | 4.00 |
| ATOM | 519 | HB1 | PHE | 137 | −8.017 | 0.000 | 10.841 | 1.00 | 0.00 | 0.053 | 0.00 | 0.00 |
| ATOM | 520 | HB2 | PHE | 137 | −8.158 | −1.667 | 10.406 | 1.00 | 0.00 | 0.053 | 0.00 | 0.00 |
| ATOM | 521 | CG | PHE | 137 | −8.652 | −1.240 | 12.365 | 1.00 | 0.00 | 0.000 | 7.26 | 0.60 |
| ATOM | 522 | CD1 | PHE | 137 | −8.659 | −2.573 | 12.743 | 1.00 | 0.00 | −0.127 | 10.80 | 0.60 |
| ATOM | 523 | HD1 | PHE | 137 | −8.659 | −3.350 | 11.978 | 1.00 | 0.00 | 0.127 | 0.00 | 0.00 |
| ATOM | 524 | CD2 | PHE | 137 | −8.650 | −0.254 | 13.355 | 1.00 | 0.00 | −0.127 | 10.80 | 0.60 |
| ATOM | 525 | HD2 | PHE | 137 | −8.644 | 0.798 | 13.070 | 1.00 | 0.00 | 0.127 | 0.00 | 0.00 |
| ATOM | 526 | CE1 | PHE | 137 | −8.665 | −2.931 | 14.089 | 1.00 | 0.00 | −0.127 | 10.80 | 0.60 |
| ATOM | 527 | HE1 | PHE | 137 | −8.671 | −3.983 | 14.374 | 1.00 | 0.00 | 0.127 | 0.00 | 0.00 |
| ATOM | 528 | CE2 | PHE | 137 | −8.655 | −0.606 | 14.706 | 1.00 | 0.00 | −0.127 | 10.80 | 0.60 |
| ATOM | 529 | HE2 | PHE | 137 | −8.652 | 0.168 | 15.473 | 1.00 | 0.00 | 0.127 | 0.00 | 0.00 |
| ATOM | 530 | CZ | PHE | 137 | −8.663 | −1.945 | 15.068 | 1.00 | 0.00 | −0.127 | 10.80 | 0.60 |
| ATOM | 531 | HZ | PHE | 137 | −8.667 | −2.224 | 16.121 | 1.00 | 0.00 | 0.127 | 0.00 | 0.00 |
| ATOM | 532 | C | PHE | 137 | −9.959 | −0.021 | 8.976 | 1.00 | 0.00 | 0.396 | 9.82 | 4.00 |
| ATOM | 533 | O | PHE | 137 | −8.897 | −0.115 | 8.359 | 1.00 | 0.00 | −0.396 | 8.17 | −17.40 |
| ATOM | 534 | N | PRO | 138 | −11.023 | 0.606 | 8.460 | 1.00 | 0.00 | −0.422 | 9.00 | −17.40 |
| ATOM | 535 | CD | PRO | 138 | −12.373 | 0.786 | 9.018 | 1.00 | 0.00 | 0.105 | 12.77 | 4.00 |
| ATOM | 536 | HD1 | PRO | 138 | −12.332 | 1.052 | 10.074 | 1.00 | 0.00 | 0.053 | 0.00 | 0.00 |
| ATOM | 537 | HD2 | PRO | 138 | −12.962 | −0.126 | 8.931 | 1.00 | 0.00 | 0.053 | 0.00 | 0.00 |
| ATOM | 538 | CA | PRO | 138 | −10.925 | 1.185 | 7.120 | 1.00 | 0.00 | 0.158 | 9.40 | 4.00 |
| ATOM | 539 | HA | PRO | 138 | −10.529 | 0.405 | 6.469 | 1.00 | 0.00 | 0.053 | 0.00 | 0.00 |
| ATOM | 540 | CB | PRO | 138 | −12.361 | 1.606 | 6.813 | 1.00 | 0.00 | −0.106 | 12.77 | 4.00 |
| ATOM | 541 | HB1 | PRO | 138 | −12.906 | 0.807 | 6.309 | 1.00 | 0.00 | 0.053 | 0.00 | 0.00 |
| ATOM | 542 | HB2 | PRO | 138 | −12.384 | 2.482 | 6.166 | 1.00 | 0.00 | 0.053 | 0.00 | 0.00 |
| ATOM | 543 | CG | PRO | 138 | −12.921 | 1.897 | 8.173 | 1.00 | 0.00 | −0.106 | 12.77 | 4.00 |
| ATOM | 544 | HG1 | PRO | 138 | −14.010 | 1.892 | 8.156 | 1.00 | 0.00 | 0.053 | 0.00 | 0.00 |
| ATOM | 545 | HG2 | PRO | 138 | −12.595 | 2.874 | 8.529 | 1.00 | 0.00 | 0.053 | 0.00 | 0.00 |
| ATOM | 546 | C | PRO | 138 | −9.984 | 2.372 | 7.224 | 1.00 | 0.00 | 0.396 | 9.82 | 4.00 |
| ATOM | 547 | O | PRO | 138 | −10.013 | 3.101 | 8.209 | 1.00 | 0.00 | −0.396 | 8.17 | −17.40 |
| ATOM | 548 | N | ILE | 139 | −9.152 | 2.555 | 6.210 | 1.00 | 0.00 | −0.650 | 9.00 | −17.40 |
| ATOM | 549 | HN | ILE | 139 | −9.190 | 1.906 | 5.411 | 1.00 | 0.00 | 0.440 | 0.00 | 0.00 |
| ATOM | 550 | CA | ILE | 139 | −8.184 | 3.643 | 6.187 | 1.00 | 0.00 | 0.158 | 9.40 | 4.00 |
| ATOM | 551 | HA | ILE | 139 | −8.067 | 4.046 | 7.193 | 1.00 | 0.00 | 0.053 | 0.00 | 0.00 |
| ATOM | 552 | CB | ILE | 139 | −6.803 | 3.121 | 5.713 | 1.00 | 0.00 | −0.053 | 9.40 | 4.00 |
| ATOM | 553 | HB | ILE | 139 | −6.905 | 2.523 | 4.807 | 1.00 | 0.00 | 0.053 | 0.00 | 0.00 |
| ATOM | 554 | CG2 | ILE | 139 | −5.838 | 4.309 | 5.405 | 1.00 | 0.00 | −0.159 | 16.15 | 4.00 |
| ATOM | 555 | HG2 | ILE | 139 | −4.875 | 3.919 | 5.074 | 1.00 | 0.00 | 0.053 | 0.00 | 0.00 |
| ATOM | 556 | HG2 | ILE | 139 | −6.266 | 4.931 | 4.619 | 1.00 | 0.00 | 0.053 | 0.00 | 0.00 |
| ATOM | 557 | HG2 | ILE | 139 | −5.697 | 4.906 | 6.305 | 1.00 | 0.00 | 0.053 | 0.00 | 0.00 |
| ATOM | 558 | CG1 | ILE | 139 | −6.225 | 2.184 | 6.782 | 1.00 | 0.00 | −0.106 | 12.77 | 4.00 |
| ATOM | 559 | HG1 | ILE | 139 | −6.971 | 1.518 | 7.214 | 1.00 | 0.00 | 0.053 | 0.00 | 0.00 |
| ATOM | 560 | HG1 | ILE | 139 | −5.783 | 2.717 | 7.623 | 1.00 | 0.00 | 0.053 | 0.00 | 0.00 |
| ATOM | 561 | CD1 | ILE | 139 | −5.138 | 1.288 | 6.254 | 1.00 | 0.00 | −0.159 | 16.15 | 4.00 |
| ATOM | 562 | HD1 | ILE | 139 | −4.770 | 0.649 | 7.057 | 1.00 | 0.00 | 0.053 | 0.00 | 0.00 |
| ATOM | 563 | HD1 | ILE | 139 | −5.535 | 0.667 | 5.451 | 1.00 | 0.00 | 0.053 | 0.00 | 0.00 |
| ATOM | 564 | HD1 | ILE | 139 | −4.319 | 1.896 | 5.870 | 1.00 | 0.00 | 0.053 | 0.00 | 0.00 |
| ATOM | 565 | C | ILE | 139 | −8.676 | 4.738 | 5.256 | 1.00 | 0.00 | 0.396 | 9.82 | 4.00 |
| ATOM | 566 | O | ILE | 139 | −8.926 | 4.509 | 4.069 | 1.00 | 0.00 | −0.396 | 8.17 | −17.40 |
| ATOM | 567 | N | ILE | 140 | −8.835 | 5.930 | 5.805 | 1.00 | 0.00 | −0.650 | 9.00 | −17.40 |
| ATOM | 568 | HN | ILE | 140 | −8.630 | 6.056 | 6.806 | 1.00 | 0.00 | 0.440 | 0.00 | 0.00 |
| ATOM | 569 | CA | ILE | 140 | −9.292 | 7.063 | 5.024 | 1.00 | 0.00 | 0.158 | 9.40 | 4.00 |
| ATOM | 570 | HA | ILE | 140 | −9.516 | 6.664 | 4.034 | 1.00 | 0.00 | 0.053 | 0.00 | 0.00 |
| ATOM | 571 | CB | ILE | 140 | −10.554 | 7.708 | 5.676 | 1.00 | 0.00 | −0.053 | 9.40 | 4.00 |
| ATOM | 572 | HB | ILE | 140 | −10.397 | 7.869 | 6.742 | 1.00 | 0.00 | 0.053 | 0.00 | 0.00 |
| ATOM | 573 | CG2 | ILE | 140 | −10.683 | 9.043 | 5.037 | 1.00 | 0.00 | −0.159 | 16.15 | 4.00 |
| ATOM | 574 | HG2 | ILE | 140 | −11.746 | 9.475 | 5.506 | 1.00 | 0.00 | 0.053 | 0.00 | 0.00 |
| ATOM | 575 | HG2 | ILE | 140 | −10.015 | 9.715 | 5.170 | 1.00 | 0.00 | 0.053 | 0.00 | 0.00 |
| ATOM | 576 | HG2 | ILE | 140 | −11.050 | 8.902 | 3.972 | 1.00 | 0.00 | 0.053 | 0.00 | 0.00 |
| ATOM | 577 | CG1 | ILE | 140 | −11.779 | 6.797 | 5.488 | 1.00 | 0.00 | −0.106 | 12.77 | 4.00 |
| ATOM | 578 | HG1 | ILE | 140 | −11.837 | 6.516 | 4.436 | 1.00 | 0.00 | 0.053 | 0.00 | 0.00 |
| ATOM | 579 | HG1 | ILE | 140 | −12.667 | 7.353 | 5.787 | 1.00 | 0.00 | 0.053 | 0.00 | 0.00 |
| ATOM | 580 | CD1 | ILE | 140 | −11.744 | 5.537 | 6.290 | 1.00 | 0.00 | −0.159 | 16.15 | 4.00 |
| ATOM | 581 | HD1 | ILE | 140 | −12.646 | 4.956 | 6.095 | 1.00 | 0.00 | 0.053 | 0.00 | 0.00 |
| ATOM | 582 | HD1 | ILE | 140 | −10.868 | 4.951 | 6.009 | 1.00 | 0.00 | 0.053 | 0.00 | 0.00 |
| ATOM | 583 | HD1 | ILE | 140 | −11.692 | 5.782 | 7.350 | 1.00 | 0.00 | 0.053 | 0.00 | 0.00 |
| ATOM | 584 | C | ILE | 140 | −8.134 | 8.051 | 5.021 | 1.00 | 0.00 | 0.396 | 9.82 | 4.00 |
| ATOM | 585 | O | ILE | 140 | −7.722 | 8.535 | 6.076 | 1.00 | 0.00 | −0.396 | 8.17 | −17.40 |
| ATOM | 586 | N | VAL | 141 | −7.583 | 8.315 | 3.841 | 1.00 | 0.00 | −0.650 | 9.00 | −17.40 |
| ATOM | 587 | HN | VAL | 141 | −7.947 | 7.843 | 3.001 | 1.00 | 0.00 | 0.440 | 0.00 | 0.00 |
| ATOM | 588 | CA | VAL | 141 | −6.476 | 9.255 | 3.704 | 1.00 | 0.00 | 0.158 | 9.40 | 4.00 |
| ATOM | 589 | HA | VAL | 141 | −5.980 | 9.347 | 4.670 | 1.00 | 0.00 | 0.053 | 0.00 | 0.00 |
| ATOM | 590 | CB | VAL | 141 | −5.456 | 8.780 | 2.633 | 1.00 | 0.00 | −0.053 | 9.40 | 4.00 |
| ATOM | 591 | HB | VAL | 141 | −5.894 | 8.853 | 1.637 | 1.00 | 0.00 | 0.053 | 0.00 | 0.00 |
| ATOM | 592 | CG1 | VAL | 141 | −4.182 | 9.658 | 2.682 | 1.00 | 0.00 | −0.159 | 16.15 | 4.00 |
| ATOM | 593 | HG1 | VAL | 141 | −3.474 | 9.315 | 1.926 | 1.00 | 0.00 | 0.053 | 0.00 | 0.00 |
| ATOM | 594 | HG1 | VAL | 141 | −4.448 | 10.696 | 2.485 | 1.00 | 0.00 | 0.053 | 0.00 | 0.00 |
| ATOM | 595 | HG1 | VAL | 141 | −3.724 | 9.581 | 3.668 | 1.00 | 0.00 | 0.053 | 0.00 | 0.00 |
| ATOM | 596 | CG2 | VAL | 141 | −5.109 | 7.307 | 2.852 | 1.00 | 0.00 | −0.159 | 16.15 | 4.00 |
| ATOM | 597 | HG2 | VAL | 141 | −4.393 | 6.985 | 2.095 | 1.00 | 0.00 | 0.053 | 0.00 | 0.00 |
| ATOM | 598 | HG2 | VAL | 141 | −4.671 | 7.179 | 3.842 | 1.00 | 0.00 | 0.053 | 0.00 | 0.00 |
| ATOM | 599 | HG2 | VAL | 141 | −6.014 | 6.704 | 2.774 | 1.00 | 0.00 | 0.053 | 0.00 | 0.00 |
| ATOM | 600 | C | VAL | 141 | −7.038 | 10.601 | 3.262 | 1.00 | 0.00 | 0.396 | 9.82 | 4.00 |
| ATOM | 601 | O | VAL | 141 | −7.539 | 10.729 | 2.137 | 1.00 | 0.00 | −0.396 | 8.17 | −17.40 |
| ATOM | 602 | N | SER | 142 | −6.984 | 11.600 | 4.137 | 1.00 | 0.00 | −0.650 | 9.00 | −17.40 |
| ATOM | 603 | HN | SER | 142 | −6.594 | 11.436 | 5.076 | 1.00 | 0.00 | 0.440 | 0.00 | 0.00 |
| ATOM | 604 | CA | SER | 142 | −7.475 | 12.931 | 3.775 | 1.00 | 0.00 | 0.158 | 9.40 | 4.00 |
| ATOM | 605 | HA | SER | 142 | −8.200 | 12.773 | 2.976 | 1.00 | 0.00 | 0.053 | 0.00 | 0.00 |
| ATOM | 606 | CB | SER | 142 | −8.148 | 13.608 | 4.978 | 1.00 | 0.00 | 0.007 | 12.77 | 4.00 |
| ATOM | 607 | HB1 | SER | 142 | −7.418 | 13.920 | 5.725 | 1.00 | 0.00 | 0.053 | 0.00 | 0.00 |
| ATOM | 608 | HB2 | SER | 142 | −8.851 | 12.940 | 5.475 | 1.00 | 0.00 | 0.053 | 0.00 | 0.00 |
| ATOM | 609 | OG | SER | 142 | −8.877 | 14.772 | 4.590 | 1.00 | 0.00 | −0.537 | 11.04 | −17.40 |
| ATOM | 610 | HG | SER | 142 | −8.260 | 15.595 | 4.639 | 1.00 | 0.00 | 0.424 | 0.00 | 0.00 |
| ATOM | 611 | C | SER | 142 | −6.252 | 13.715 | 3.317 | 1.00 | 0.00 | 0.396 | 9.82 | 4.00 |
| ATOM | 612 | O | SER | 142 | −5.303 | 13.911 | 4.079 | 1.00 | 0.00 | −0.396 | 8.17 | −17.40 |
| ATOM | 613 | N | GLN | 143 | −6.261 | 14.140 | 2.061 | 1.00 | 0.00 | −0.650 | 9.00 | −17.40 |
| ATOM | 614 | HN | GLN | 143 | −7.071 | 13.938 | 1.458 | 1.00 | 0.00 | 0.440 | 0.00 | 0.00 |
| ATOM | 615 | CA | GLN | 143 | −5.134 | 14.889 | 1.528 | 1.00 | 0.00 | 0.158 | 9.40 | 4.00 |
| ATOM | 616 | HA | GLN | 143 | −4.285 | 14.863 | 2.211 | 1.00 | 0.00 | 0.053 | 0.00 | 0.00 |
| ATOM | 617 | CB | GLN | 143 | −4.669 | 14.296 | 0.191 | 1.00 | 0.00 | −0.106 | 12.77 | 4.00 |
| ATOM | 618 | HB1 | GLN | 143 | −5.280 | 14.628 | −0.647 | 1.00 | 0.00 | 0.053 | 0.00 | 0.00 |
| ATOM | 619 | HB2 | GLN | 143 | −4.705 | 13.206 | 0.183 | 1.00 | 0.00 | 0.053 | 0.00 | 0.00 |
| ATOM | 620 | CG | GLN | 143 | −3.231 | 14.679 | −0.148 | 1.00 | 0.00 | −0.106 | 12.77 | 4.00 |
| ATOM | 621 | HG1 | GLN | 143 | −2.485 | 14.008 | 0.279 | 1.00 | 0.00 | 0.053 | 0.00 | 0.00 |
| ATOM | 622 | HG2 | GLN | 143 | −2.947 | 15.672 | 0.199 | 1.00 | 0.00 | 0.053 | 0.00 | 0.00 |
| ATOM | 623 | CD | GLN | 143 | −2.937 | 14.689 | −1.637 | 1.00 | 0.00 | 0.396 | 9.82 | 4.00 |
| ATOM | 624 | OE1 | GLN | 143 | −3.668 | 14.114 | −2.440 | 1.00 | 0.00 | −0.396 | 8.17 | −17.40 |
| ATOM | 625 | NE2 | GLN | 143 | −1.846 | 15.347 | −2.008 | 1.00 | 0.00 | −0.879 | 13.25 | −17.40 |
| ATOM | 626 | HE2 | GLN | 143 | −1.261 | 15.815 | −1.301 | 1.00 | 0.00 | 0.440 | 0.00 | 0.00 |
| ATOM | 627 | HE2 | GLN | 143 | −1.583 | 15.389 | −3.004 | 1.00 | 0.00 | 0.440 | 0.00 | 0.00 |
| ATOM | 628 | C | GLN | 143 | −5.500 | 16.345 | 1.302 | 1.00 | 0.00 | 0.396 | 9.82 | 4.00 |
| ATOM | 629 | O | GLN | 143 | −6.510 | 16.653 | 0.666 | 1.00 | 0.00 | −0.396 | 8.17 | −17.40 |
| ATOM | 630 | N | ASP | 144P | −4.671 | 17.236 | 1.827 | 1.00 | 0.00 | −0.650 | 9.00 | −17.40 |
| ATOM | 631 | HN | ASP | 144P | −3.863 | 16.891 | 2.366 | 1.00 | 0.00 | 0.440 | 0.00 | 0.00 |
| ATOM | 632 | CA | ASP | 144P | −4.839 | 18.682 | 1.683 | 1.00 | 0.00 | 0.158 | 9.40 | 4.00 |
| ATOM | 633 | HA | ASP | 144P | −5.833 | 18.921 | 1.305 | 1.00 | 0.00 | 0.053 | 0.00 | 0.00 |
| ATOM | 634 | CB | ASP | 144P | −4.588 | 19.322 | 3.061 | 1.00 | 0.00 | −0.336 | 12.77 | 4.00 |
| ATOM | 635 | HB1 | ASP | 144P | −3.586 | 19.142 | 3.452 | 1.00 | 0.00 | 0.053 | 0.00 | 0.00 |
| ATOM | 636 | HB2 | ASP | 144P | −5.261 | 18.964 | 3.840 | 1.00 | 0.00 | 0.053 | 0.00 | 0.00 |
| ATOM | 637 | CG | ASP | 144P | −4.746 | 20.830 | 3.072 | 1.00 | 0.00 | 0.297 | 9.82 | 4.00 |
| ATOM | 638 | OD1 | ASP | 144P | −5.409 | 21.398 | 2.188 | 1.00 | 0.00 | −0.534 | 8.17 | −18.95 |
| ATOM | 639 | OD2 | ASP | 144P | −4.214 | 21.461 | 4.011 | 1.00 | 0.00 | −0.534 | 8.17 | −18.95 |
| ATOM | 640 | C | ASP | 144P | −3.738 | 19.087 | 0.679 | 1.00 | 0.00 | 0.396 | 9.82 | 4.00 |
| ATOM | 641 | O | ASP | 144P | −3.009 | 18.221 | 0.187 | 1.00 | 0.00 | −0.396 | 8.17 | −17.40 |
| ATOM | 642 | N | CYS | 145 | −3.655 | 20.371 | 0.343 | 1.00 | 0.00 | −0.650 | 9.00 | −17.40 |
| ATOM | 643 | HN | CYS | 145 | −4.370 | 21.025 | 0.690 | 1.00 | 0.00 | 0.440 | 0.00 | 0.00 |
| ATOM | 644 | CA | CYS | 145 | −2.577 | 20.882 | −0.506 | 1.00 | 0.00 | 0.158 | 9.40 | 4.00 |
| ATOM | 645 | HA | CYS | 145 | −2.598 | 21.967 | −0.408 | 1.00 | 0.00 | 0.053 | 0.00 | 0.00 |
| ATOM | 646 | C | CYS | 145 | −2.535 | 20.626 | −2.013 | 1.00 | 0.00 | 0.396 | 9.82 | 4.00 |
| ATOM | 647 | O | CYS | 145 | −1.862 | 21.357 | −2.720 | 1.00 | 0.00 | −0.396 | 8.17 | −17.40 |
| ATOM | 648 | CB | CYS | 145 | −1.248 | 20.432 | 0.106 | 1.00 | 0.00 | −0.041 | 12.77 | 4.00 |
| ATOM | 649 | HB1 | CYS | 145 | −0.410 | 20.986 | −0.317 | 1.00 | 0.00 | 0.053 | 0.00 | 0.00 |
| ATOM | 650 | HB2 | CYS | 145 | −1.069 | 19.372 | −0.075 | 1.00 | 0.00 | 0.053 | 0.00 | 0.00 |
| ATOM | 651 | SG | CYS | 145 | −1.231 | 20.697 | 1.915 | 1.00 | 0.00 | −0.065 | 19.93 | −6.40 |
| ATOM | 652 | N | GLY | 146 | −3.205 | 19.593 | −2.506 | 1.00 | 0.00 | −0.650 | 9.00 | −17.40 |
| ATOM | 653 | HN | GLY | 146 | −3.736 | 18.973 | −1.879 | 1.00 | 0.00 | 0.440 | 0.00 | 0.00 |
| ATOM | 654 | CA | GLY | 146 | −3.185 | 19.340 | −3.942 | 1.00 | 0.00 | 0.105 | 9.40 | 4.00 |
| ATOM | 655 | HA1 | GLY | 146 | −3.506 | 20.255 | −4.439 | 1.00 | 0.00 | 0.053 | 0.00 | 0.00 |
| ATOM | 656 | HA2 | GLY | 146 | −3.872 | 18.517 | −4.138 | 1.00 | 0.00 | 0.053 | 0.00 | 0.00 |
| ATOM | 657 | C | GLY | 146 | −1.852 | 18.949 | −4.579 | 1.00 | 0.00 | 0.396 | 9.82 | 4.00 |
| ATOM | 658 | O | GLY | 146 | −1.664 | 19.142 | −5.779 | 1.00 | 0.00 | −0.396 | 8.17 | −17.40 |
| ATOM | 659 | N | HIS | 147S | −0.926 | 18.396 | −3.796 | 1.00 | 0.00 | −0.650 | 9.00 | −17.40 |
| ATOM | 660 | HN | HIS | 147S | −1.141 | 18.252 | −2.799 | 1.00 | 0.00 | 0.440 | 0.00 | 0.00 |
| ATOM | 661 | CA | HIS | 147S | 0.390 | 17.983 | −4.302 | 1.00 | 0.00 | 0.158 | 9.40 | 4.00 |
| ATOM | 662 | HA | HIS | 147S | 0.797 | 18.822 | −4.866 | 1.00 | 0.00 | 0.053 | 0.00 | 0.00 |
| ATOM | 663 | CB | HIS | 147S | 1.282 | 17.627 | −3.105 | 1.00 | 0.00 | −0.106 | 12.77 | 4.00 |
| ATOM | 664 | HB1 | HIS | 147S | 0.898 | 16.711 | −2.655 | 1.00 | 0.00 | 0.053 | 0.00 | 0.00 |
| ATOM | 665 | HB2 | HIS | 147S | 1.243 | 18.452 | −2.394 | 1.00 | 0.00 | 0.053 | 0.00 | 0.00 |
| ATOM | 666 | CG | HIS | 147S | 2.721 | 17.393 | −3.448 | 1.00 | 0.00 | −0.050 | 7.26 | 0.60 |
| ATOM | 667 | CD2 | HIS | 147S | 3.744 | 18.260 | −3.636 | 1.00 | 0.00 | −0.177 | 10.80 | 0.60 |
| ATOM | 668 | HD2 | HIS | 147S | 3.682 | 19.347 | −3.612 | 1.00 | 0.00 | 0.127 | 0.00 | 0.00 |
| ATOM | 669 | ND1 | HIS | 147S | 3.263 | 16.131 | −3.573 | 1.00 | 0.00 | 0.207 | 9.25 | −17.40 |
| ATOM | 670 | HD1 | HIS | 147S | 2.740 | 15.247 | −3.487 | 1.00 | 0.00 | 0.393 | 0.00 | 0.00 |
| ATOM | 671 | CE1 | HIS | 147S | 4.557 | 16.230 | −3.821 | 1.00 | 0.00 | −0.227 | 10.80 | 0.60 |
| ATOM | 672 | HE1 | HIS | 147S | 5.244 | 15.396 | −3.967 | 1.00 | 0.00 | 0.127 | 0.00 | 0.00 |
| ATOM | 673 | NE2 | HIS | 147S | 4.875 | 17.513 | −3.863 | 1.00 | 0.00 | 0.207 | 9.25 | −17.40 |
| ATOM | 674 | HE2 | HIS | 147S | 5.817 | 17.890 | −4.038 | 1.00 | 0.00 | 0.393 | 0.00 | 0.00 |
| ATOM | 675 | C | HIS | 147S | 0.170 | 16.762 | −5.193 | 1.00 | 0.00 | 0.396 | 9.82 | 4.00 |
| ATOM | 676 | O | HIS | 147S | −0.197 | 15.698 | −4.695 | 1.00 | 0.00 | −0.396 | 8.17 | −17.40 |
| ATOM | 677 | N | GLU | 148 | 0.398 | 16.918 | −6.500 | 1.00 | 0.00 | −0.650 | 9.00 | −17.40 |
| ATOM | 678 | HN | GLU | 148 | 0.746 | 17.827 | −6.837 | 1.00 | 0.00 | 0.440 | 0.00 | 0.00 |
| ATOM | 679 | CA | GLU | 148 | 0.171 | 15.842 | −7.478 | 1.00 | 0.00 | 0.158 | 9.40 | 4.00 |
| ATOM | 680 | HA | GLU | 148 | −0.897 | 15.627 | −7.496 | 1.00 | 0.00 | 0.053 | 0.00 | 0.00 |
| ATOM | 681 | CB | GLU | 148 | 0.540 | 16.321 | −8.892 | 1.00 | 0.00 | −0.106 | 12.77 | 4.00 |
| ATOM | 682 | HB1 | GLU | 148 | 1.601 | 16.568 | −8.902 | 1.00 | 0.00 | 0.053 | 0.00 | 0.00 |
| ATOM | 683 | HB2 | GLU | 148 | −0.063 | 17.199 | −9.122 | 1.00 | 0.00 | 0.053 | 0.00 | 0.00 |
| ATOM | 684 | CG | GLU | 148 | 0.297 | 15.290 | −10.003 | 1.00 | 0.00 | −0.106 | 12.77 | 4.00 |
| ATOM | 685 | HG1 | GLU | 148 | 0.951 | 14.422 | −9.921 | 1.00 | 0.00 | 0.053 | 0.00 | 0.00 |
| ATOM | 686 | HG2 | GLU | 148 | 0.461 | 15.699 | −11.000 | 1.00 | 0.00 | 0.053 | 0.00 | 0.00 |
| ATOM | 687 | CD | GLU | 148 | −1.126 | 14.748 | −9.999 | 1.00 | 0.00 | 0.399 | 9.82 | 4.00 |
| ATOM | 688 | OE1 | GLU | 148 | −2.051 | 15.519 | −9.657 | 1.00 | 0.00 | −0.396 | 8.17 | −18.95 |
| ATOM | 689 | OE2 | GLU | 148 | −1.325 | 13.556 | −10.349 | 1.00 | 0.00 | −0.427 | 8.17 | −18.95 |
| ATOM | 690 | HE2 | GLU | 148 | −2.332 | 13.347 | −10.304 | 1.00 | 0.00 | 0.424 | 0.00 | 0.00 |
| ATOM | 691 | C | GLU | 148 | 0.885 | 14.523 | −7.208 | 1.00 | 0.00 | 0.396 | 9.82 | 4.00 |
| ATOM | 692 | O | GLU | 148 | 0.268 | 13.455 | −7.277 | 1.00 | 0.00 | −0.396 | 8.17 | −17.40 |
| ATOM | 693 | N | GLU | 149 | 2.178 | 14.586 | −6.917 | 1.00 | 0.00 | −0.650 | 9.00 | −17.40 |
| ATOM | 694 | HN | GLU | 149 | 2.653 | 15.498 | −6.871 | 1.00 | 0.00 | 0.440 | 0.00 | 0.00 |
| ATOM | 695 | CA | GLU | 149 | 2.920 | 13.364 | −6.664 | 1.00 | 0.00 | 0.158 | 9.40 | 4.00 |
| ATOM | 696 | HA | GLU | 149 | 2.841 | 12.671 | −7.502 | 1.00 | 0.00 | 0.053 | 0.00 | 0.00 |
| ATOM | 697 | CB | GLU | 149 | 4.410 | 13.648 | −6.496 | 1.00 | 0.00 | −0.106 | 12.77 | 4.00 |
| ATOM | 698 | HB1 | GLU | 149 | 4.617 | 14.366 | −5.703 | 1.00 | 0.00 | 0.053 | 0.00 | 0.00 |
| ATOM | 699 | HB2 | GLU | 149 | 4.867 | 14.058 | −7.397 | 1.00 | 0.00 | 0.053 | 0.00 | 0.00 |
| ATOM | 700 | CG | GLU | 149 | 5.208 | 12.413 | −6.154 | 1.00 | 0.00 | −0.106 | 12.77 | 4.00 |
| ATOM | 701 | HG1 | GLU | 149 | 5.104 | 11.631 | −6.908 | 1.00 | 0.00 | 0.053 | 0.00 | 0.00 |
| ATOM | 702 | HG2 | GLU | 149 | 4.901 | 11.971 | −5.206 | 1.00 | 0.00 | 0.053 | 0.00 | 0.00 |
| ATOM | 703 | CD | GLU | 149 | 6.690 | 12.701 | −6.034 | 1.00 | 0.00 | 0.399 | 9.82 | 4.00 |
| ATOM | 704 | OE1 | GLU | 149 | 7.412 | 11.901 | −5.395 | 1.00 | 0.00 | −0.396 | 8.17 | −18.95 |
| ATOM | 705 | OE2 | GLU | 149 | 7.132 | 13.729 | −6.589 | 1.00 | 0.00 | −0.427 | 8.17 | −18.95 |
| ATOM | 706 | HE2 | GLU | 149 | 8.147 | 13.794 | −6.433 | 1.00 | 0.00 | 0.424 | 0.00 | 0.00 |
| ATOM | 707 | C | GLU | 149 | 2.402 | 12.647 | −5.428 | 1.00 | 0.00 | 0.396 | 9.82 | 4.00 |
| ATOM | 708 | O | GLU | 149 | 2.257 | 11.430 | −5.432 | 1.00 | 0.00 | −0.396 | 8.17 | −17.40 |
| ATOM | 709 | N | THR | 150 | 2.119 | 13.394 | −4.367 | 1.00 | 0.00 | −0.650 | 9.00 | −17.40 |
| ATOM | 710 | HN | THR | 150 | 2.258 | 14.414 | −4.393 | 1.00 | 0.00 | 0.440 | 0.00 | 0.00 |
| ATOM | 711 | CA | THR | 150 | 1.609 | 12.749 | −3.169 | 1.00 | 0.00 | 0.158 | 9.40 | 4.00 |
| ATOM | 712 | HA | THR | 150 | 2.308 | 11.970 | −2.866 | 1.00 | 0.00 | 0.053 | 0.00 | 0.00 |
| ATOM | 713 | CB | THR | 150 | 1.492 | 13.738 | −2.003 | 1.00 | 0.00 | 0.060 | 9.40 | 4.00 |
| ATOM | 714 | HB | THR | 150 | 0.818 | 14.547 | −2.283 | 1.00 | 0.00 | 0.053 | 0.00 | 0.00 |
| ATOM | 715 | OG1 | THR | 150 | 2.797 | 14.274 | −1.677 | 1.00 | 0.00 | −0.537 | 11.04 | −17.40 |
| ATOM | 716 | HG1 | THR | 150 | 3.466 | 14.024 | −2.420 | 1.00 | 0.00 | 0.424 | 0.00 | 0.00 |
| ATOM | 717 | CG2 | THR | 150 | 0.938 | 13.010 | −0.774 | 1.00 | 0.00 | −0.159 | 16.15 | 4.00 |
| ATOM | 718 | HG2 | THR | 150 | 0.853 | 13.710 | −0.055 | 1.00 | 0.00 | 0.053 | 0.00 | 0.00 |
| ATOM | 719 | HG2 | THR | 150 | −0.045 | 12.601 | −1.006 | 1.00 | 0.00 | 0.053 | 0.00 | 0.00 |
| ATOM | 720 | HG2 | THR | 150 | 1.611 | 12.198 | −0.498 | 1.00 | 0.00 | 0.053 | 0.00 | 0.00 |
| ATOM | 721 | C | THR | 150 | 0.232 | 12.133 | −3.486 | 1.00 | 0.00 | 0.396 | 9.82 | 4.00 |
| ATOM | 722 | O | THR | 150 | −0.128 | 11.083 | −2.939 | 1.00 | 0.00 | −0.396 | 8.17 | −17.40 |
| ATOM | 723 | N | ALA | 151 | −0.528 | 12.785 | −4.369 | 1.00 | 0.00 | −0.650 | 9.00 | −17.40 |
| ATOM | 724 | HN | ALA | 151 | −0.178 | 13.658 | −4.788 | 1.00 | 0.00 | 0.440 | 0.00 | 0.00 |
| ATOM | 725 | CA | ALA | 151 | −1.847 | 12.284 | −4.755 | 1.00 | 0.00 | 0.158 | 9.40 | 4.00 |
| ATOM | 726 | HA | ALA | 151 | −2.471 | 12.117 | −3.877 | 1.00 | 0.00 | 0.053 | 0.00 | 0.00 |
| ATOM | 727 | CB | ALA | 151 | −2.570 | 13.315 | −5.634 | 1.00 | 0.00 | −0.159 | 16.15 | 4.00 |
| ATOM | 728 | HB1 | ALA | 151 | −3.550 | 12.930 | −5.915 | 1.00 | 0.00 | 0.053 | 0.00 | 0.00 |
| ATOM | 729 | HB2 | ALA | 151 | −2.690 | 14.245 | −5.080 | 1.00 | 0.00 | 0.053 | 0.00 | 0.00 |
| ATOM | 730 | HB3 | ALA | 151 | −1.982 | 13.502 | −6.533 | 1.00 | 0.00 | 0.053 | 0.00 | 0.00 |
| ATOM | 731 | C | ALA | 151 | −1.727 | 10.961 | −5.508 | 1.00 | 0.00 | 0.396 | 9.82 | 4.00 |
| ATOM | 732 | O | ALA | 151 | −2.497 | 10.018 | −5.265 | 1.00 | 0.00 | −0.396 | 8.17 | −17.40 |
| ATOM | 733 | N | GLN | 152 | −0.764 | 10.888 | −6.426 | 1.00 | 0.00 | −0.650 | 9.00 | −17.40 |
| ATOM | 734 | HN | GLN | 152 | −0.147 | 11.698 | −6.582 | 1.00 | 0.00 | 0.440 | 0.00 | 0.00 |
| ATOM | 735 | CA | GLN | 152 | −0.571 | 9.665 | −7.219 | 1.00 | 0.00 | 0.158 | 9.40 | 4.00 |
| ATOM | 736 | HA | GLN | 152 | −1.501 | 9.393 | −7.717 | 1.00 | 0.00 | 0.053 | 0.00 | 0.00 |
| ATOM | 737 | CB | GLN | 152 | 0.472 | 9.904 | −8.325 | 1.00 | 0.00 | −0.106 | 12.77 | 4.00 |
| ATOM | 738 | HB1 | GLN | 152 | 0.564 | 9.053 | −9.001 | 1.00 | 0.00 | 0.053 | 0.00 | 0.00 |
| ATOM | 739 | HB2 | GLN | 152 | 1.469 | 10.087 | −7.925 | 1.00 | 0.00 | 0.053 | 0.00 | 0.00 |
| ATOM | 740 | CG | GLN | 152 | 0.130 | 11.116 | −9.201 | 1.00 | 0.00 | −0.106 | 12.77 | 4.00 |
| ATOM | 741 | HG1 | GLN | 152 | 0.010 | 11.985 | −8.554 | 1.00 | 0.00 | 0.053 | 0.00 | 0.00 |
| ATOM | 742 | HG2 | GLN | 152 | −0.797 | 10.904 | −9.733 | 1.00 | 0.00 | 0.053 | 0.00 | 0.00 |
| ATOM | 743 | CD | GLN | 152 | 1.194 | 11.450 | −10.237 | 1.00 | 0.00 | 0.396 | 9.82 | 4.00 |
| ATOM | 744 | OE1 | GLN | 152 | 2.395 | 11.221 | −10.024 | 1.00 | 0.00 | −0.396 | 8.17 | −17.40 |
| ATOM | 745 | NE2 | GLN | 152 | 0.761 | 12.029 | −11.357 | 1.00 | 0.00 | −0.879 | 13.25 | −17.40 |
| ATOM | 746 | HE2 | GLN | 152 | −0.245 | 12.200 | −11.493 | 1.00 | 0.00 | 0.440 | 0.00 | 0.00 |
| ATOM | 747 | HE2 | GLN | 152 | 1.433 | 12.305 | −12.087 | 1.00 | 0.00 | 0.440 | 0.00 | 0.00 |
| ATOM | 748 | C | GLN | 152 | −0.141 | 8.518 | −6.311 | 1.00 | 0.00 | 0.396 | 9.82 | 4.00 |
| ATOM | 749 | O | GLN | 152 | −0.603 | 7.386 | −6.458 | 1.00 | 0.00 | −0.396 | 8.17 | −17.40 |
| ATOM | 750 | N | VAL | 153 | 0.752 | 8.812 | −5.375 | 1.00 | 0.00 | −0.650 | 9.00 | −17.40 |
| ATOM | 751 | HN | VAL | 153 | 1.126 | 9.769 | −5.320 | 1.00 | 0.00 | 0.440 | 0.00 | 0.00 |
| ATOM | 752 | CA | VAL | 153 | 1.214 | 7.813 | −4.426 | 1.00 | 0.00 | 0.158 | 9.40 | 4.00 |
| ATOM | 753 | HA | VAL | 153 | 1.734 | 7.002 | −4.937 | 1.00 | 0.00 | 0.053 | 0.00 | 0.00 |
| ATOM | 754 | CB | VAL | 153 | 2.187 | 8.436 | −3.416 | 1.00 | 0.00 | −0.053 | 9.40 | 4.00 |
| ATOM | 755 | HB | VAL | 153 | 1.756 | 9.337 | −2.981 | 1.00 | 0.00 | 0.053 | 0.00 | 0.00 |
| ATOM | 756 | CG1 | VAL | 153 | 2.493 | 7.438 | −2.276 | 1.00 | 0.00 | −0.159 | 16.15 | 4.00 |
| ATOM | 757 | HG1 | VAL | 153 | 3.184 | 7.895 | −1.569 | 1.00 | 0.00 | 0.053 | 0.00 | 0.00 |
| ATOM | 758 | HG1 | VAL | 153 | 1.567 | 7.177 | −1.763 | 1.00 | 0.00 | 0.053 | 0.00 | 0.00 |
| ATOM | 759 | HG1 | VAL | 153 | 2.943 | 6.536 | −2.693 | 1.00 | 0.00 | 0.053 | 0.00 | 0.00 |
| ATOM | 760 | CG2 | VAL | 153 | 3.465 | 8.855 | −4.146 | 1.00 | 0.00 | −0.159 | 16.15 | 4.00 |
| ATOM | 761 | HG2 | VAL | 153 | 4.161 | 9.298 | −3.435 | 1.00 | 0.00 | 0.053 | 0.00 | 0.00 |
| ATOM | 762 | HG2 | VAL | 153 | 3.924 | 7.980 | −4.607 | 1.00 | 0.00 | 0.053 | 0.00 | 0.00 |
| ATOM | 763 | HG2 | VAL | 153 | 3.220 | 9.584 | −4.918 | 1.00 | 0.00 | 0.053 | 0.00 | 0.00 |
| ATOM | 764 | C | VAL | 153 | 0.041 | 7.210 | −3.656 | 1.00 | 0.00 | 0.396 | 9.82 | 4.00 |
| ATOM | 765 | O | VAL | 153 | −0.055 | 5.980 | −3.508 | 1.00 | 0.00 | −0.396 | 8.17 | −17.40 |
| ATOM | 766 | N | ILE | 154 | −0.854 | 8.075 | −3.184 | 1.00 | 0.00 | −0.650 | 9.00 | −17.40 |
| ATOM | 767 | HN | ILE | 154 | −0.723 | 9.080 | −3.364 | 1.00 | 0.00 | 0.440 | 0.00 | 0.00 |
| ATOM | 768 | CA | ILE | 154 | −2.011 | 7.635 | −2.421 | 1.00 | 0.00 | 0.158 | 9.40 | 4.00 |
| ATOM | 769 | HA | ILE | 154 | −1.657 | 7.020 | −1.594 | 1.00 | 0.00 | 0.053 | 0.00 | 0.00 |
| ATOM | 770 | CB | ILE | 154 | −2.786 | 8.834 | −1.809 | 1.00 | 0.00 | −0.053 | 9.40 | 4.00 |
| ATOM | 771 | HB | ILE | 154 | −3.097 | 9.521 | −2.596 | 1.00 | 0.00 | 0.053 | 0.00 | 0.00 |
| ATOM | 772 | CG2 | ILE | 154 | −4.026 | 8.339 | −1.072 | 1.00 | 0.00 | −0.159 | 16.15 | 4.00 |
| ATOM | 773 | HG2 | ILE | 154 | −4.560 | 9.188 | −0.648 | 1.00 | 0.00 | 0.053 | 0.00 | 0.00 |
| ATOM | 774 | HG2 | ILE | 154 | −4.677 | 7.812 | −1.770 | 1.00 | 0.00 | 0.053 | 0.00 | 0.00 |
| ATOM | 775 | HG2 | ILE | 154 | −3.727 | 7.661 | −0.272 | 1.00 | 0.00 | 0.053 | 0.00 | 0.00 |
| ATOM | 776 | CG1 | ILE | 154 | −1.887 | 9.587 | −0.816 | 1.00 | 0.00 | −0.106 | 12.77 | 4.00 |
| ATOM | 777 | HG1 | ILE | 154 | −0.922 | 9.757 | −1.294 | 1.00 | 0.00 | 0.053 | 0.00 | 0.00 |
| ATOM | 778 | HG1 | ILE | 154 | −1.773 | 8.969 | 0.073 | 1.00 | 0.00 | 0.053 | 0.00 | 0.00 |
| ATOM | 779 | CD1 | ILE | 154 | −2.428 | 10.962 | −0.360 | 1.00 | 0.00 | −0.159 | 16.15 | 4.00 |
| ATOM | 780 | HD1 | ILE | 154 | −1.725 | 11.416 | 0.337 | 1.00 | 0.00 | 0.053 | 0.00 | 0.00 |
| ATOM | 781 | HD1 | ILE | 154 | −2.548 | 11.611 | −1.228 | 1.00 | 0.00 | 0.053 | 0.00 | 0.00 |
| ATOM | 782 | HD1 | ILE | 154 | −3.392 | 10.829 | 0.129 | 1.00 | 0.00 | 0.053 | 0.00 | 0.00 |
| ATOM | 783 | C | ILE | 154 | −2.907 | 6.831 | −3.345 | 1.00 | 0.00 | 0.396 | 9.82 | 4.00 |
| ATOM | 784 | O | ILE | 154 | −3.307 | 5.733 | −2.994 | 1.00 | 0.00 | −0.396 | 8.17 | −17.40 |
| ATOM | 785 | N | ALA | 155 | −3.191 | 7.371 | −4.532 | 1.00 | 0.00 | −0.650 | 9.00 | −17.40 |
| ATOM | 786 | HN | ALA | 155 | −2.803 | 8.298 | −4.758 | 1.00 | 0.00 | 0.440 | 0.00 | 0.00 |
| ATOM | 787 | CA | ALA | 155 | −4.035 | 6.698 | −5.533 | 1.00 | 0.00 | 0.158 | 9.40 | 4.00 |
| ATOM | 788 | HA | ALA | 155 | −5.065 | 6.576 | −5.200 | 1.00 | 0.00 | 0.053 | 0.00 | 0.00 |
| ATOM | 789 | CB | ALA | 155 | −4.103 | 7.551 | −6.821 | 1.00 | 0.00 | −0.159 | 16.15 | 4.00 |
| ATOM | 790 | HB1 | ALA | 155 | −4.728 | 7.047 | −7.558 | 1.00 | 0.00 | 0.053 | 0.00 | 0.00 |
| ATOM | 791 | HB2 | ALA | 155 | −4.529 | 8.527 | −6.589 | 1.00 | 0.00 | 0.053 | 0.00 | 0.00 |
| ATOM | 792 | HB3 | ALA | 155 | −3.099 | 7.680 | −7.226 | 1.00 | 0.00 | 0.053 | 0.00 | 0.00 |
| ATOM | 793 | C | ALA | 155 | −3.530 | 5.288 | −5.881 | 1.00 | 0.00 | 0.396 | 9.82 | 4.00 |
| ATOM | 794 | O | ALA | 155 | −4.325 | 4.381 | −6.150 | 1.00 | 0.00 | −0.396 | 8.17 | −17.40 |
| ATOM | 795 | N | SER | 156 | −2.210 | 5.110 | −5.888 | 1.00 | 0.00 | −0.650 | 9.00 | −17.40 |
| ATOM | 796 | HN | SER | 156 | −1.592 | 5.901 | −5.655 | 1.00 | 0.00 | 0.440 | 0.00 | 0.00 |
| ATOM | 797 | CA | SER | 156 | −1.621 | 3.818 | −6.219 | 1.00 | 0.00 | 0.158 | 9.40 | 4.00 |
| ATOM | 798 | HA | SER | 156 | −1.944 | 3.540 | −7.223 | 1.00 | 0.00 | 0.053 | 0.00 | 0.00 |
| ATOM | 799 | CB | SER | 156 | −0.088 | 3.917 | −6.282 | 1.00 | 0.00 | 0.007 | 12.77 | 4.00 |
| ATOM | 800 | HB1 | SER | 156 | 0.256 | 4.860 | −6.706 | 1.00 | 0.00 | 0.053 | 0.00 | 0.00 |
| ATOM | 801 | HB2 | SER | 156 | 0.360 | 3.132 | −6.892 | 1.00 | 0.00 | 0.053 | 0.00 | 0.00 |
| ATOM | 802 | OG | SER | 156 | 0.523 | 3.816 | −5.002 | 1.00 | 0.00 | −0.537 | 11.04 | −17.40 |
| ATOM | 803 | HG | SER | 156 | 0.601 | 4.753 | −4.584 | 1.00 | 0.00 | 0.424 | 0.00 | 0.00 |
| ATOM | 804 | C | SER | 156 | −2.024 | 2.687 | −5.268 | 1.00 | 0.00 | 0.396 | 9.82 | 4.00 |
| ATOM | 805 | O | SER | 156 | −1.841 | 1.504 | −5.599 | 1.00 | 0.00 | −0.396 | 8.17 | −17.40 |
| ATOM | 806 | N | TYR | 157 | −2.557 | 3.026 | −4.093 | 1.00 | 0.00 | −0.650 | 9.00 | −17.40 |
| ATOM | 807 | HN | TYR | 157 | −2.659 | 4.019 | −3.840 | 1.00 | 0.00 | 0.440 | 0.00 | 0.00 |
| ATOM | 808 | CA | TYR | 157 | −2.995 | 1.986 | −3.167 | 1.00 | 0.00 | 0.158 | 9.40 | 4.00 |
| ATOM | 809 | HA | TYR | 157 | −2.283 | 1.160 | −3.144 | 1.00 | 0.00 | 0.053 | 0.00 | 0.00 |
| ATOM | 810 | CB | TYR | 157 | −3.048 | 2.502 | −1.725 | 1.00 | 0.00 | −0.106 | 12.77 | 4.00 |
| ATOM | 811 | HB1 | TYR | 157 | −3.593 | 1.817 | −1.075 | 1.00 | 0.00 | 0.053 | 0.00 | 0.00 |
| ATOM | 812 | HB2 | TYR | 157 | −3.543 | 3.471 | −1.665 | 1.00 | 0.00 | 0.053 | 0.00 | 0.00 |
| ATOM | 813 | CG | TYR | 157 | −1.671 | 2.666 | −1.147 | 1.00 | 0.00 | 0.000 | 7.26 | 0.60 |
| ATOM | 814 | CD1 | TYR | 157 | −0.894 | 3.777 | −1.454 | 1.00 | 0.00 | −0.127 | 10.80 | 0.60 |
| ATOM | 815 | HD1 | TYR | 157 | −1.310 | 4.559 | −2.089 | 1.00 | 0.00 | 0.127 | 0.00 | 0.00 |
| ATOM | 816 | CE1 | TYR | 157 | 0.411 | 3.914 | −0.966 | 1.00 | 0.00 | −0.127 | 10.80 | 0.60 |
| ATOM | 817 | HE1 | TYR | 157 | 1.001 | 4.795 | −1.216 | 1.00 | 0.00 | 0.127 | 0.00 | 0.00 |
| ATOM | 818 | CD2 | TYR | 157 | −1.116 | 1.674 | −0.332 | 1.00 | 0.00 | −0.127 | 10.80 | 0.60 |
| ATOM | 819 | HD2 | TYR | 157 | −1.708 | 0.795 | −0.077 | 1.00 | 0.00 | 0.127 | 0.00 | 0.00 |
| ATOM | 820 | CE2 | TYR | 157 | 0.187 | 1.792 | 0.159 | 1.00 | 0.00 | −0.127 | 10.80 | 0.60 |
| ATOM | 821 | HE2 | TYR | 157 | 0.609 | 1.010 | 0.790 | 1.00 | 0.00 | 0.127 | 0.00 | 0.00 |
| ATOM | 822 | CZ | TYR | 157 | 0.944 | 2.921 | −0.165 | 1.00 | 0.00 | 0.026 | 7.26 | 0.60 |
| ATOM | 823 | OH | TYR | 157 | 2.238 | 3.024 | 0.313 | 1.00 | 0.00 | −0.451 | 10.94 | −17.40 |
| ATOM | 824 | HH | TYR | 157 | 2.631 | 3.937 | 0.046 | 1.00 | 0.00 | 0.424 | 0.00 | 0.00 |
| ATOM | 825 | C | TYR | 157 | −4.349 | 1.427 | −3.579 | 1.00 | 0.00 | 0.396 | 9.82 | 4.00 |
| ATOM | 826 | O | TYR | 157 | −4.793 | 0.400 | −3.067 | 1.00 | 0.00 | −0.396 | 8.17 | −17.40 |
| ATOM | 827 | N | GLY | 158 | −4.992 | 2.094 | −4.531 | 1.00 | 0.00 | −0.650 | 9.00 | −17.40 |
| ATOM | 828 | HN | GLY | 158 | −4.577 | 2.947 | −4.933 | 1.00 | 0.00 | 0.440 | 0.00 | 0.00 |
| ATOM | 829 | CA | GLY | 158 | −6.277 | 1.619 | −5.003 | 1.00 | 0.00 | 0.105 | 9.40 | 4.00 |
| ATOM | 830 | HA1 | GLY | 158 | −6.213 | 0.651 | −5.501 | 1.00 | 0.00 | 0.053 | 0.00 | 0.00 |
| ATOM | 831 | HA2 | GLY | 158 | −6.739 | 2.295 | −5.722 | 1.00 | 0.00 | 0.053 | 0.00 | 0.00 |
| ATOM | 832 | C | GLY | 158 | −7.286 | 1.446 | −3.886 | 1.00 | 0.00 | 0.396 | 9.82 | 4.00 |
| ATOM | 833 | O | GLY | 158 | −7.398 | 2.310 | −3.012 | 1.00 | 0.00 | −0.396 | 8.17 | −17.40 |
| ATOM | 834 | N | SER | 159 | −8.008 | 0.327 | −3.906 | 1.00 | 0.00 | −0.650 | 9.00 | −17.40 |
| ATOM | 835 | HN | SER | 159 | −7.833 | −0.364 | −4.649 | 1.00 | 0.00 | 0.440 | 0.00 | 0.00 |
| ATOM | 836 | CA | SER | 159 | −9.043 | 0.047 | −2.905 | 1.00 | 0.00 | 0.158 | 9.40 | 4.00 |
| ATOM | 837 | HA | SER | 159 | −9.730 | 0.892 | −2.893 | 1.00 | 0.00 | 0.053 | 0.00 | 0.00 |
| ATOM | 838 | CB | SER | 159 | −9.866 | −1.184 | −3.302 | 1.00 | 0.00 | 0.007 | 12.77 | 4.00 |
| ATOM | 839 | HB1 | SER | 159 | −10.182 | −1.136 | −4.344 | 1.00 | 0.00 | 0.053 | 0.00 | 0.00 |
| ATOM | 840 | HB2 | SER | 159 | −10.766 | −1.278 | −2.695 | 1.00 | 0.00 | 0.053 | 0.00 | 0.00 |
| ATOM | 841 | OG | SER | 159 | −9.121 | −2.387 | −3.142 | 1.00 | 0.00 | −0.537 | 11.04 | −17.40 |
| ATOM | 842 | HG | SER | 159 | −9.761 | −3.159 | −2.911 | 1.00 | 0.00 | 0.424 | 0.00 | 0.00 |
| ATOM | 843 | C | SER | 159 | −8.566 | −0.156 | −1.473 | 1.00 | 0.00 | 0.396 | 9.82 | 4.00 |
| ATOM | 844 | O | SER | 159 | −9.391 | −0.219 | −0.564 | 1.00 | 0.00 | −0.396 | 8.17 | −17.40 |
| ATOM | 845 | N | ALA | 160 | −7.260 | −0.269 | −1.254 | 1.00 | 0.00 | −0.650 | 9.00 | −17.40 |
| ATOM | 846 | HN | ALA | 160 | −6.597 | −0.222 | −2.041 | 1.00 | 0.00 | 0.440 | 0.00 | 0.00 |
| ATOM | 847 | CA | ALA | 160 | −6.770 | −0.460 | 0.107 | 1.00 | 0.00 | 0.158 | 9.40 | 4.00 |
| ATOM | 848 | HA | ALA | 160 | −7.243 | −1.337 | 0.547 | 1.00 | 0.00 | 0.053 | 0.00 | 0.00 |
| ATOM | 849 | CB | ALA | 160 | −5.261 | −0.725 | 0.108 | 1.00 | 0.00 | −0.159 | 16.15 | 4.00 |
| ATOM | 850 | HB1 | ALA | 160 | −4.916 | −0.864 | 1.132 | 1.00 | 0.00 | 0.053 | 0.00 | 0.00 |
| ATOM | 851 | HB2 | ALA | 160 | −5.049 | −1.623 | −0.471 | 1.00 | 0.00 | 0.053 | 0.00 | 0.00 |
| ATOM | 852 | HB3 | ALA | 160 | −4.742 | 0.124 | −0.336 | 1.00 | 0.00 | 0.053 | 0.00 | 0.00 |
| ATOM | 853 | C | ALA | 160 | −7.105 | 0.773 | 0.953 | 1.00 | 0.00 | 0.396 | 9.82 | 4.00 |
| ATOM | 854 | O | ALA | 160 | −7.188 | 0.685 | 2.185 | 1.00 | 0.00 | −0.396 | 8.17 | −17.40 |
| ATOM | 855 | N | VAL | 161 | −7.304 | 1.921 | 0.305 | 1.00 | 0.00 | −0.650 | 9.00 | −17.40 |
| ATOM | 856 | HN | VAL | 161 | −7.207 | 1.960 | −0.719 | 1.00 | 0.00 | 0.440 | 0.00 | 0.00 |
| ATOM | 857 | CA | VAL | 161 | −7.660 | 3.122 | 1.056 | 1.00 | 0.00 | 0.158 | 9.40 | 4.00 |
| ATOM | 858 | HA | VAL | 161 | −8.029 | 2.889 | 2.055 | 1.00 | 0.00 | 0.053 | 0.00 | 0.00 |
| ATOM | 859 | CB | VAL | 161 | −6.442 | 4.058 | 1.247 | 1.00 | 0.00 | −0.053 | 9.40 | 4.00 |
| ATOM | 860 | HB | VAL | 161 | −6.736 | 4.946 | 1.805 | 1.00 | 0.00 | 0.053 | 0.00 | 0.00 |
| ATOM | 861 | CG1 | VAL | 161 | −5.349 | 3.341 | 2.011 | 1.00 | 0.00 | −0.159 | 16.15 | 4.00 |
| ATOM | 862 | HG1 | VAL | 161 | −4.497 | 4.008 | 2.139 | 1.00 | 0.00 | 0.053 | 0.00 | 0.00 |
| ATOM | 863 | HG1 | VAL | 161 | −5.725 | 3.040 | 2.988 | 1.00 | 0.00 | 0.053 | 0.00 | 0.00 |
| ATOM | 864 | HG1 | VAL | 161 | −5.036 | 2.456 | 1.455 | 1.00 | 0.00 | 0.053 | 0.00 | 0.00 |
| ATOM | 865 | CG2 | VAL | 161 | −5.930 | 4.532 | −0.132 | 1.00 | 0.00 | −0.159 | 16.15 | 4.00 |
| ATOM | 866 | HG2 | VAL | 161 | −5.072 | 5.190 | 0.003 | 1.00 | 0.00 | 0.053 | 0.00 | 0.00 |
| ATOM | 867 | HG2 | VAL | 161 | −5.632 | 3.667 | −0.727 | 1.00 | 0.00 | 0.053 | 0.00 | 0.00 |
| ATOM | 868 | HG2 | VAL | 161 | −6.723 | 5.071 | −0.649 | 1.00 | 0.00 | 0.053 | 0.00 | 0.00 |
| ATOM | 869 | C | VAL | 161 | −8.746 | 3.926 | 0.370 | 1.00 | 0.00 | 0.396 | 9.82 | 4.00 |
| ATOM | 870 | O | VAL | 161 | −9.109 | 3.651 | −0.783 | 1.00 | 0.00 | −0.396 | 8.17 | −17.40 |
| ATOM | 871 | N | THR | 162 | −9.273 | 4.913 | 1.096 | 1.00 | 0.00 | −0.650 | 9.00 | −17.40 |
| ATOM | 872 | HN | THR | 162 | −8.955 | 5.035 | 2.068 | 1.00 | 0.00 | 0.440 | 0.00 | 0.00 |
| ATOM | 873 | CA | THR | 162 | −10.273 | 5.818 | 0.572 | 1.00 | 0.00 | 0.158 | 9.40 | 4.00 |
| ATOM | 874 | HA | THR | 162 | −10.582 | 5.525 | −0.431 | 1.00 | 0.00 | 0.053 | 0.00 | 0.00 |
| ATOM | 875 | CB | THR | 162 | −11.545 | 5.881 | 1.468 | 1.00 | 0.00 | 0.060 | 9.40 | 4.00 |
| ATOM | 876 | HB | THR | 162 | −11.269 | 6.107 | 2.498 | 1.00 | 0.00 | 0.053 | 0.00 | 0.00 |
| ATOM | 877 | OG1 | THR | 162 | −12.230 | 4.616 | 1.429 | 1.00 | 0.00 | −0.537 | 11.04 | −17.40 |
| ATOM | 878 | HG1 | THR | 162 | −13.247 | 4.776 | 1.404 | 1.00 | 0.00 | 0.424 | 0.00 | 0.00 |
| ATOM | 879 | CG2 | THR | 162 | −12.508 | 6.985 | 0.956 | 1.00 | 0.00 | −0.159 | 16.15 | 4.00 |
| ATOM | 880 | HG2 | THR | 162 | −13.393 | 7.018 | 1.591 | 1.00 | 0.00 | 0.053 | 0.00 | 0.00 |
| ATOM | 881 | HG2 | THR | 162 | −12.003 | 7.950 | 0.985 | 1.00 | 0.00 | 0.053 | 0.00 | 0.00 |
| ATOM | 882 | HG2 | THR | 162 | −12.804 | 6.762 | −0.068 | 1.00 | 0.00 | 0.053 | 0.00 | 0.00 |
| ATOM | 883 | C | THR | 162 | −9.544 | 7.165 | 0.579 | 1.00 | 0.00 | 0.396 | 9.82 | 4.00 |
| ATOM | 884 | O | THR | 162 | −9.136 | 7.655 | 1.636 | 1.00 | 0.00 | −0.396 | 8.17 | −17.40 |
| ATOM | 885 | N | HIS | 163S | −9.375 | 7.749 | −0.604 | 1.00 | 0.00 | −0.650 | 9.00 | −17.40 |
| ATOM | 886 | HN | HIS | 163S | −9.765 | 7.294 | −1.442 | 1.00 | 0.00 | 0.440 | 0.00 | 0.00 |
| ATOM | 887 | CA | HIS | 163S | −8.659 | 9.006 | −0.759 | 1.00 | 0.00 | 0.158 | 9.40 | 4.00 |
| ATOM | 888 | HA | HIS | 163S | −8.021 | 9.196 | 0.103 | 1.00 | 0.00 | 0.053 | 0.00 | 0.00 |
| ATOM | 889 | CB | HIS | 163S | −7.777 | 8.910 | −2.016 | 1.00 | 0.00 | −0.106 | 12.77 | 4.00 |
| ATOM | 890 | HB1 | HIS | 163S | −8.429 | 8.845 | −2.887 | 1.00 | 0.00 | 0.053 | 0.00 | 0.00 |
| ATOM | 891 | HB2 | HIS | 163S | −7.158 | 8.016 | −1.932 | 1.00 | 0.00 | 0.053 | 0.00 | 0.00 |
| ATOM | 892 | CG | HIS | 163S | −6.861 | 10.075 | −2.223 | 1.00 | 0.00 | −0.050 | 7.26 | 0.60 |
| ATOM | 893 | CD2 | HIS | 163S | −6.486 | 11.080 | −1.395 | 1.00 | 0.00 | −0.177 | 10.80 | 0.60 |
| ATOM | 894 | HD2 | HIS | 163S | −6.822 | 11.232 | −0.370 | 1.00 | 0.00 | 0.127 | 0.00 | 0.00 |
| ATOM | 895 | ND1 | HIS | 163S | −6.183 | 10.284 | −3.409 | 1.00 | 0.00 | 0.207 | 9.25 | −17.40 |
| ATOM | 896 | HD1 | HIS | 163S | −6.250 | 9.687 | −4.246 | 1.00 | 0.00 | 0.393 | 0.00 | 0.00 |
| ATOM | 897 | CE1 | HIS | 163S | −5.434 | 11.370 | −3.302 | 1.00 | 0.00 | −0.227 | 10.80 | 0.60 |
| ATOM | 898 | HE1 | HIS | 163S | −4.789 | 11.780 | −4.080 | 1.00 | 0.00 | 0.127 | 0.00 | 0.00 |
| ATOM | 899 | NE2 | HIS | 163S | −5.601 | 11.872 | −2.091 | 1.00 | 0.00 | 0.207 | 9.25 | −17.40 |
| ATOM | 900 | HE2 | HIS | 163S | −5.142 | 12.719 | −1.727 | 1.00 | 0.00 | 0.393 | 0.00 | 0.00 |
| ATOM | 901 | C | HIS | 163S | −9.636 | 10.171 | −0.896 | 1.00 | 0.00 | 0.396 | 9.82 | 4.00 |
| ATOM | 902 | O | HIS | 163S | −10.426 | 10.191 | −1.846 | 1.00 | 0.00 | −0.396 | 8.17 | −17.40 |
| ATOM | 903 | N | ILE | 164 | −9.625 | 11.102 | 0.063 | 1.00 | 0.00 | −0.650 | 9.00 | −17.40 |
| ATOM | 904 | HN | ILE | 164 | −8.993 | 10.983 | 0.868 | 1.00 | 0.00 | 0.440 | 0.00 | 0.00 |
| ATOM | 905 | CA | ILE | 164 | −10.485 | 12.285 | 0.002 | 1.00 | 0.00 | 0.158 | 9.40 | 4.00 |
| ATOM | 906 | HA | ILE | 164 | −11.073 | 12.230 | −0.913 | 1.00 | 0.00 | 0.053 | 0.00 | 0.00 |
| ATOM | 907 | CB | ILE | 164 | −11.521 | 12.349 | 1.186 | 1.00 | 0.00 | −0.053 | 9.40 | 4.00 |
| ATOM | 908 | HB | ILE | 164 | −12.091 | 13.273 | 1.100 | 1.00 | 0.00 | 0.053 | 0.00 | 0.00 |
| ATOM | 909 | CG2 | ILE | 164 | −12.448 | 11.146 | 1.087 | 1.00 | 0.00 | −0.159 | 16.15 | 4.00 |
| ATOM | 910 | HG2 | ILE | 164 | −13.171 | 11.176 | 1.901 | 1.00 | 0.00 | 0.053 | 0.00 | 0.00 |
| ATOM | 911 | HG2 | ILE | 164 | −12.974 | 11.169 | 0.132 | 1.00 | 0.00 | 0.053 | 0.00 | 0.00 |
| ATOM | 912 | HG2 | ILE | 164 | −11.862 | 10.229 | 1.156 | 1.00 | 0.00 | 0.053 | 0.00 | 0.00 |
| ATOM | 913 | CG1 | ILE | 164 | −10.807 | 12.397 | 2.542 | 1.00 | 0.00 | −0.106 | 12.77 | 4.00 |
| ATOM | 914 | HG1 | ILE | 164 | −10.101 | 13.228 | 2.528 | 1.00 | 0.00 | 0.053 | 0.00 | 0.00 |
| ATOM | 915 | HG1 | ILE | 164 | −10.283 | 11.451 | 2.687 | 1.00 | 0.00 | 0.053 | 0.00 | 0.00 |
| ATOM | 916 | CD1 | ILE | 164 | −11.739 | 12.599 | 3.736 | 1.00 | 0.00 | −0.159 | 16.15 | 4.00 |
| ATOM | 917 | HD1 | ILE | 164 | −11.153 | 12.621 | 4.655 | 1.00 | 0.00 | 0.053 | 0.00 | 0.00 |
| ATOM | 918 | HD1 | ILE | 164 | −12.274 | 13.541 | 3.624 | 1.00 | 0.00 | 0.053 | 0.00 | 0.00 |
| ATOM | 919 | HD1 | ILE | 164 | −12.454 | 11.778 | 3.781 | 1.00 | 0.00 | 0.053 | 0.00 | 0.00 |
| ATOM | 920 | C | ILE | 164 | −9.592 | 13.522 | −0.001 | 1.00 | 0.00 | 0.396 | 9.82 | 4.00 |
| ATOM | 921 | O | ILE | 164 | −8.460 | 13.513 | 0.536 | 1.00 | 0.00 | −0.396 | 8.17 | −17.40 |
| ATOM | 922 | N | ARG | 165G | −10.093 | 14.586 | −0.616 | 1.00 | 0.00 | −0.650 | 9.00 | −17.40 |
| ATOM | 923 | HN | ARG | 165G | −11.049 | 14.549 | −0.996 | 1.00 | 0.00 | 0.440 | 0.00 | 0.00 |
| ATOM | 924 | CA | ARG | 165G | −9.310 | 15.801 | −0.761 | 1.00 | 0.00 | 0.158 | 9.40 | 4.00 |
| ATOM | 925 | HA | ARG | 165G | −8.359 | 15.658 | −0.247 | 1.00 | 0.00 | 0.053 | 0.00 | 0.00 |
| ATOM | 926 | CB | ARG | 165G | −9.009 | 16.010 | −2.246 | 1.00 | 0.00 | −0.106 | 12.77 | 4.00 |
| ATOM | 927 | HB1 | ARG | 165G | −8.288 | 16.810 | −2.415 | 1.00 | 0.00 | 0.053 | 0.00 | 0.00 |
| ATOM | 928 | HB2 | ARG | 165G | −9.899 | 16.271 | −2.818 | 1.00 | 0.00 | 0.053 | 0.00 | 0.00 |
| ATOM | 929 | CG | ARG | 165G | −8.431 | 14.769 | −2.898 | 1.00 | 0.00 | −0.106 | 12.77 | 4.00 |
| ATOM | 930 | HG1 | ARG | 165G | −9.118 | 13.924 | −2.861 | 1.00 | 0.00 | 0.053 | 0.00 | 0.00 |
| ATOM | 931 | HG2 | ARG | 165G | −7.509 | 14.440 | −2.418 | 1.00 | 0.00 | 0.053 | 0.00 | 0.00 |
| ATOM | 932 | CD | ARG | 165G | −8.098 | 14.990 | −4.363 | 1.00 | 0.00 | 0.374 | 12.77 | 4.00 |
| ATOM | 933 | HD1 | ARG | 165G | −7.201 | 15.599 | −4.480 | 1.00 | 0.00 | 0.053 | 0.00 | 0.00 |
| ATOM | 934 | HD2 | ARG | 165G | −8.909 | 15.498 | −4.883 | 1.00 | 0.00 | 0.053 | 0.00 | 0.00 |
| ATOM | 935 | NE | ARG | 165G | −7.860 | 13.721 | −5.054 | 1.00 | 0.00 | −0.819 | 9.00 | −24.67 |
| ATOM | 936 | HE | ARG | 165G | −8.424 | 12.907 | −4.770 | 1.00 | 0.00 | 0.407 | 0.00 | 0.00 |
| ATOM | 937 | CZ | ARG | 165G | −6.963 | 13.544 | −6.026 | 1.00 | 0.00 | 0.796 | 6.95 | 4.00 |
| ATOM | 938 | NH1 | ARG | 165G | −6.202 | 14.555 | −6.433 | 1.00 | 0.00 | −0.746 | 9.00 | −24.67 |
| ATOM | 939 | HH1 | ARG | 165G | −5.511 | 14.410 | −7.184 | 1.00 | 0.00 | 0.407 | 0.00 | 0.00 |
| ATOM | 940 | HH1 | ARG | 165G | −6.302 | 15.483 | −5.998 | 1.00 | 0.00 | 0.407 | 0.00 | 0.00 |
| ATOM | 941 | NH2 | ARG | 165G | −6.827 | 12.352 | −6.598 | 1.00 | 0.00 | −0.746 | 9.00 | −24.67 |
| ATOM | 942 | HH2 | ARG | 165G | −6.134 | 12.216 | −7.349 | 1.00 | 0.00 | 0.407 | 0.00 | 0.00 |
| ATOM | 943 | HH2 | ARG | 165G | −7.414 | 11.563 | −6.291 | 1.00 | 0.00 | 0.407 | 0.00 | 0.00 |
| ATOM | 944 | C | ARG | 165G | −9.960 | 17.033 | −0.186 | 1.00 | 0.00 | 0.396 | 9.82 | 4.00 |
| ATOM | 945 | O | ARG | 165G | −11.082 | 17.395 | −0.562 | 1.00 | 0.00 | −0.396 | 8.17 | −17.40 |
| ATOM | 946 | N | GLN | 166 | −9.244 | 17.672 | 0.735 | 1.00 | 0.00 | −0.650 | 9.00 | −17.40 |
| ATOM | 947 | HN | GLN | 166 | −8.322 | 17.294 | 0.998 | 1.00 | 0.00 | 0.440 | 0.00 | 0.00 |
| ATOM | 948 | CA | GLN | 166 | −9.715 | 18.883 | 1.383 | 1.00 | 0.00 | 0.158 | 9.40 | 4.00 |
| ATOM | 949 | HA | GLN | 166 | −10.586 | 18.683 | 2.007 | 1.00 | 0.00 | 0.053 | 0.00 | 0.00 |
| ATOM | 950 | CB | GLN | 166 | −8.588 | 19.437 | 2.263 | 1.00 | 0.00 | −0.106 | 12.77 | 4.00 |
| ATOM | 951 | HB1 | GLN | 166 | −7.898 | 19.988 | 1.624 | 1.00 | 0.00 | 0.053 | 0.00 | 0.00 |
| ATOM | 952 | HB2 | GLN | 166 | −8.084 | 18.596 | 2.739 | 1.00 | 0.00 | 0.053 | 0.00 | 0.00 |
| ATOM | 953 | CG | GLN | 166 | −9.043 | 20.376 | 3.360 | 1.00 | 0.00 | −0.106 | 12.77 | 4.00 |
| ATOM | 954 | HG1 | GLN | 166 | −8.295 | 20.469 | 4.147 | 1.00 | 0.00 | 0.053 | 0.00 | 0.00 |
| ATOM | 955 | HG2 | GLN | 166 | −9.963 | 20.030 | 3.830 | 1.00 | 0.00 | 0.053 | 0.00 | 0.00 |
| ATOM | 956 | CD | GLN | 166 | −9.308 | 21.767 | 2.832 | 1.00 | 0.00 | 0.396 | 9.82 | 4.00 |
| ATOM | 957 | OE1 | GLN | 166 | −10.463 | 22.202 | 2.684 | 1.00 | 0.00 | −0.396 | 8.17 | −17.40 |
| ATOM | 958 | NE2 | GLN | 166 | −8.231 | 22.478 | 2.527 | 1.00 | 0.00 | −0.879 | 13.25 | −17.40 |
| ATOM | 959 | HE2 | GLN | 166 | −7.292 | 22.076 | 2.666 | 1.00 | 0.00 | 0.440 | 0.00 | 0.00 |
| ATOM | 960 | HE2 | GLN | 166 | −8.332 | 23.431 | 2.150 | 1.00 | 0.00 | 0.440 | 0.00 | 0.00 |
| ATOM | 961 | C | GLN | 166 | −10.054 | 19.791 | 0.188 | 1.00 | 0.00 | 0.396 | 9.82 | 4.00 |
| ATOM | 962 | O | GLN | 166 | −9.222 | 20.026 | −0.679 | 1.00 | 0.00 | −0.396 | 8.17 | −17.40 |
| ATOM | 963 | N | PRO | 167 | −11.292 | 20.298 | 0.127 | 1.00 | 0.00 | −0.422 | 9.00 | −17.40 |
| ATOM | 964 | CD | PRO | 167 | −12.390 | 20.081 | 1.089 | 1.00 | 0.00 | 0.105 | 12.77 | 4.00 |
| ATOM | 965 | HD1 | PRO | 167 | −12.094 | 20.372 | 2.096 | 1.00 | 0.00 | 0.053 | 0.00 | 0.00 |
| ATOM | 966 | HD2 | PRO | 167 | −12.684 | 19.032 | 1.120 | 1.00 | 0.00 | 0.053 | 0.00 | 0.00 |
| ATOM | 967 | CA | PRO | 167 | −11.721 | 21.150 | −0.988 | 1.00 | 0.00 | 0.158 | 9.40 | 4.00 |
| ATOM | 968 | HA | PRO | 167 | −11.343 | 20.681 | −1.897 | 1.00 | 0.00 | 0.053 | 0.00 | 0.00 |
| ATOM | 969 | CB | PRO | 167 | −13.240 | 21.006 | −0.947 | 1.00 | 0.00 | −0.106 | 12.77 | 4.00 |
| ATOM | 970 | HB1 | PRO | 167 | −13.568 | 20.095 | −1.449 | 1.00 | 0.00 | 0.053 | 0.00 | 0.00 |
| ATOM | 971 | HB2 | PRO | 167 | −13.733 | 21.844 | −1.438 | 1.00 | 0.00 | 0.053 | 0.00 | 0.00 |
| ATOM | 972 | CG | PRO | 167 | −13.502 | 20.972 | 0.538 | 1.00 | 0.00 | −0.106 | 12.77 | 4.00 |
| ATOM | 973 | HG1 | PRO | 167 | −14.488 | 20.558 | 0.749 | 1.00 | 0.00 | 0.053 | 0.00 | 0.00 |
| ATOM | 974 | HG2 | PRO | 167 | −13.458 | 21.974 | 0.963 | 1.00 | 0.00 | 0.053 | 0.00 | 0.00 |
| ATOM | 975 | C | PRO | 167 | −11.297 | 22.606 | −1.090 | 1.00 | 0.00 | 0.396 | 9.82 | 4.00 |
| ATOM | 976 | O | PRO | 167 | −11.373 | 23.181 | −2.176 | 1.00 | 0.00 | −0.396 | 8.17 | −17.40 |
| ATOM | 977 | N | ASP | 168P | −10.836 | 23.207 | 0.001 | 1.00 | 0.00 | −0.650 | 9.00 | −17.40 |
| ATOM | 978 | HN | ASP | 168P | −10.731 | 22.670 | 0.874 | 1.00 | 0.00 | 0.440 | 0.00 | 0.00 |
| ATOM | 979 | CA | ASP | 168P | −10.476 | 24.624 | −0.026 | 1.00 | 0.00 | 0.158 | 9.40 | 4.00 |
| ATOM | 980 | HA | ASP | 168P | −10.814 | 25.137 | −0.927 | 1.00 | 0.00 | 0.053 | 0.00 | 0.00 |
| ATOM | 981 | CB | ASP | 168P | −11.169 | 25.325 | 1.147 | 1.00 | 0.00 | −0.336 | 12.77 | 4.00 |
| ATOM | 982 | HB1 | ASP | 168P | −10.700 | 25.098 | 2.104 | 1.00 | 0.00 | 0.053 | 0.00 | 0.00 |
| ATOM | 983 | HB2 | ASP | 168P | −12.216 | 25.039 | 1.245 | 1.00 | 0.00 | 0.053 | 0.00 | 0.00 |
| ATOM | 984 | CG | ASP | 168P | −11.153 | 26.828 | 1.015 | 1.00 | 0.00 | 0.297 | 9.82 | 4.00 |
| ATOM | 985 | OD1 | ASP | 168P | −10.592 | 27.312 | 0.018 | 1.00 | 0.00 | −0.534 | 8.17 | −18.95 |
| ATOM | 986 | OD2 | ASP | 168P | −11.699 | 27.521 | 1.904 | 1.00 | 0.00 | −0.534 | 8.17 | −18.95 |
| ATOM | 987 | C | ASP | 168P | −8.975 | 24.865 | 0.035 | 1.00 | 0.00 | 0.396 | 9.82 | 4.00 |
| ATOM | 988 | O | ASP | 168P | −8.380 | 24.809 | 1.104 | 1.00 | 0.00 | −0.396 | 8.17 | −17.40 |
| ATOM | 989 | N | LEU | 169 | −8.359 | 25.140 | −1.108 | 1.00 | 0.00 | −0.650 | 9.00 | −17.40 |
| ATOM | 990 | HN | LEU | 169 | −8.903 | 25.196 | −1.981 | 1.00 | 0.00 | 0.440 | 0.00 | 0.00 |
| ATOM | 991 | CA | LEU | 169 | −6.920 | 25.362 | −1.139 | 1.00 | 0.00 | 0.158 | 9.40 | 4.00 |
| ATOM | 992 | HA | LEU | 169 | −6.480 | 24.861 | −0.277 | 1.00 | 0.00 | 0.053 | 0.00 | 0.00 |
| ATOM | 993 | CB | LEU | 169 | −6.319 | 24.682 | −2.373 | 1.00 | 0.00 | −0.106 | 12.77 | 4.00 |
| ATOM | 994 | HB1 | LEU | 169 | −5.242 | 24.849 | −2.410 | 1.00 | 0.00 | 0.053 | 0.00 | 0.00 |
| ATOM | 995 | HB2 | LEU | 169 | −6.765 | 25.085 | −3.282 | 1.00 | 0.00 | 0.053 | 0.00 | 0.00 |
| ATOM | 996 | CG | LEU | 169 | −6.581 | 23.165 | −2.327 | 1.00 | 0.00 | −0.053 | 9.40 | 4.00 |
| ATOM | 997 | HG | LEU | 169 | −7.643 | 22.948 | −2.436 | 1.00 | 0.00 | 0.053 | 0.00 | 0.00 |
| ATOM | 998 | CD1 | LEU | 169 | −5.842 | 22.450 | −3.447 | 1.00 | 0.00 | −0.159 | 16.15 | 4.00 |
| ATOM | 999 | HD1 | LEU | 169 | −6.044 | 21.380 | −3.392 | 1.00 | 0.00 | 0.053 | 0.00 | 0.00 |
| ATOM | 1000 | HD1 | LEU | 169 | −6.180 | 22.833 | −4.410 | 1.00 | 0.00 | 0.053 | 0.00 | 0.00 |
| ATOM | 1001 | HD1 | LEU | 169 | −4.770 | 22.622 | −3.345 | 1.00 | 0.00 | 0.053 | 0.00 | 0.00 |
| ATOM | 1002 | CD2 | LEU | 169 | −6.133 | 22.623 | −0.963 | 1.00 | 0.00 | −0.159 | 16.15 | 4.00 |
| ATOM | 1003 | HD2 | LEU | 169 | −6.314 | 21.549 | −0.921 | 1.00 | 0.00 | 0.053 | 0.00 | 0.00 |
| ATOM | 1004 | HD2 | LEU | 169 | −5.069 | 22.816 | −0.826 | 1.00 | 0.00 | 0.053 | 0.00 | 0.00 |
| ATOM | 1005 | HD2 | LEU | 169 | −6.696 | 23.117 | −0.172 | 1.00 | 0.00 | 0.053 | 0.00 | 0.00 |
| ATOM | 1006 | C | LEU | 169 | −6.498 | 26.826 | −1.084 | 1.00 | 0.00 | 0.396 | 9.82 | 4.00 |
| ATOM | 1007 | O | LEU | 169 | −5.325 | 27.146 | −1.275 | 1.00 | 0.00 | −0.396 | 8.17 | −17.40 |
| ATOM | 1008 | N | SER | 170 | −7.443 | 27.710 | −0.796 | 1.00 | 0.00 | −0.650 | 9.00 | −17.40 |
| ATOM | 1009 | HN | SER | 170 | −8.403 | 27.388 | −0.610 | 1.00 | 0.00 | 0.440 | 0.00 | 0.00 |
| ATOM | 1010 | CA | SER | 170 | −7.135 | 29.133 | −0.741 | 1.00 | 0.00 | 0.158 | 9.40 | 4.00 |
| ATOM | 1011 | HA | SER | 170 | −6.692 | 29.487 | −1.672 | 1.00 | 0.00 | 0.053 | 0.00 | 0.00 |
| ATOM | 1012 | CB | SER | 170 | −8.418 | 29.934 | −0.502 | 1.00 | 0.00 | 0.007 | 12.77 | 4.00 |
| ATOM | 1013 | HB1 | SER | 170 | −9.184 | 29.624 | −1.213 | 1.00 | 0.00 | 0.053 | 0.00 | 0.00 |
| ATOM | 1014 | HB2 | SER | 170 | −8.218 | 30.997 | −0.632 | 1.00 | 0.00 | 0.053 | 0.00 | 0.00 |
| ATOM | 1015 | OG | SER | 170 | −8.888 | 29.706 | 0.817 | 1.00 | 0.00 | −0.537 | 11.04 | −17.40 |
| ATOM | 1016 | HG | SER | 170 | −9.157 | 30.602 | 1.245 | 1.00 | 0.00 | 0.424 | 0.00 | 0.00 |
| ATOM | 1017 | C | SER | 170 | −6.148 | 29.456 | 0.374 | 1.00 | 0.00 | 0.396 | 9.82 | 4.00 |
| ATOM | 1018 | O | SER | 170 | −5.988 | 28.686 | 1.318 | 1.00 | 0.00 | −0.396 | 8.17 | −17.40 |
| ATOM | 1019 | N | ASN | 171 | −5.475 | 30.598 | 0.262 | 1.00 | 0.00 | −0.650 | 9.00 | −17.40 |
| ATOM | 1020 | HN | ASN | 171 | −5.604 | 31.187 | −0.572 | 1.00 | 0.00 | 0.440 | 0.00 | 0.00 |
| ATOM | 1021 | CA | ASN | 171 | −4.556 | 31.021 | 1.310 | 1.00 | 0.00 | 0.158 | 9.40 | 4.00 |
| ATOM | 1022 | HA | ASN | 171 | −3.983 | 30.134 | 1.583 | 1.00 | 0.00 | 0.053 | 0.00 | 0.00 |
| ATOM | 1023 | CB | ASN | 171 | −3.624 | 32.138 | 0.837 | 1.00 | 0.00 | −0.106 | 12.77 | 4.00 |
| ATOM | 1024 | HB1 | ASN | 171 | −3.157 | 32.671 | 1.665 | 1.00 | 0.00 | 0.053 | 0.00 | 0.00 |
| ATOM | 1025 | HB2 | ASN | 171 | −4.142 | 32.890 | 0.242 | 1.00 | 0.00 | 0.053 | 0.00 | 0.00 |
| ATOM | 1026 | CG | ASN | 171 | −2.491 | 31.622 | −0.025 | 1.00 | 0.00 | 0.396 | 9.82 | 4.00 |
| ATOM | 1027 | OD1 | ASN | 171 | −1.953 | 30.530 | 0.210 | 1.00 | 0.00 | −0.396 | 8.17 | −17.40 |
| ATOM | 1028 | ND2 | ASN | 171 | −2.107 | 32.408 | −1.021 | 1.00 | 0.00 | −0.879 | 13.25 | −17.40 |
| ATOM | 1029 | HD2 | ASN | 171 | −2.583 | 33.307 | −1.181 | 1.00 | 0.00 | 0.440 | 0.00 | 0.00 |
| ATOM | 1030 | HD2 | ASN | 171 | −1.332 | 32.119 | −1.636 | 1.00 | 0.00 | 0.440 | 0.00 | 0.00 |
| ATOM | 1031 | C | ASN | 171 | −5.421 | 31.530 | 2.442 | 1.00 | 0.00 | 0.396 | 9.82 | 4.00 |
| ATOM | 1032 | O | ASN | 171 | −6.560 | 31.971 | 2.234 | 1.00 | 0.00 | −0.396 | 8.17 | −17.40 |
| ATOM | 1033 | N | ILE | 172 | −4.883 | 31.483 | 3.648 | 1.00 | 0.00 | −0.650 | 9.00 | −17.40 |
| ATOM | 1034 | HN | ILE | 172 | −3.917 | 31.143 | 3.766 | 1.00 | 0.00 | 0.440 | 0.00 | 0.00 |
| ATOM | 1035 | CA | ILE | 172 | −5.643 | 31.906 | 4.800 | 1.00 | 0.00 | 0.158 | 9.40 | 4.00 |
| ATOM | 1036 | HA | ILE | 172 | −6.697 | 32.048 | 4.565 | 1.00 | 0.00 | 0.053 | 0.00 | 0.00 |
| ATOM | 1037 | CB | ILE | 172 | −5.617 | 30.799 | 5.879 | 1.00 | 0.00 | −0.053 | 9.40 | 4.00 |
| ATOM | 1038 | HB | ILE | 172 | −4.592 | 30.578 | 6.180 | 1.00 | 0.00 | 0.053 | 0.00 | 0.00 |
| ATOM | 1039 | CG2 | ILE | 172 | −6.390 | 31.232 | 7.110 | 1.00 | 0.00 | −0.159 | 16.15 | 4.00 |
| ATOM | 1040 | HG2 | ILE | 172 | −6.359 | 30.438 | 7.856 | 1.00 | 0.00 | 0.053 | 0.00 | 0.00 |
| ATOM | 1041 | HG2 | ILE | 172 | −5.941 | 32.135 | 7.522 | 1.00 | 0.00 | 0.053 | 0.00 | 0.00 |
| ATOM | 1042 | HG2 | ILE | 172 | −7.425 | 31.433 | 6.837 | 1.00 | 0.00 | 0.053 | 0.00 | 0.00 |
| ATOM | 1043 | CG1 | ILE | 172 | −6.237 | 29.522 | 5.301 | 1.00 | 0.00 | −0.106 | 12.77 | 4.00 |
| ATOM | 1044 | HG1 | ILE | 172 | −5.613 | 29.180 | 4.475 | 1.00 | 0.00 | 0.053 | 0.00 | 0.00 |
| ATOM | 1045 | HG1 | ILE | 172 | −7.241 | 29.756 | 4.949 | 1.00 | 0.00 | 0.053 | 0.00 | 0.00 |
| ATOM | 1046 | CD1 | ILE | 172 | −6.348 | 28.389 | 6.295 | 1.00 | 0.00 | −0.159 | 16.15 | 4.00 |
| ATOM | 1047 | HD1 | ILE | 172 | −6.796 | 27.521 | 5.809 | 1.00 | 0.00 | 0.053 | 0.00 | 0.00 |
| ATOM | 1048 | HD1 | ILE | 172 | −5.355 | 28.127 | 6.661 | 1.00 | 0.00 | 0.053 | 0.00 | 0.00 |
| ATOM | 1049 | HD1 | ILE | 172 | −6.973 | 28.699 | 7.132 | 1.00 | 0.00 | 0.053 | 0.00 | 0.00 |
| ATOM | 1050 | C | ILE | 172 | −5.138 | 33.226 | 5.366 | 1.00 | 0.00 | 0.396 | 9.82 | 4.00 |
| ATOM | 1051 | O | ILE | 172 | −3.928 | 33.433 | 5.492 | 1.00 | 0.00 | −0.396 | 8.17 | −17.40 |
| ATOM | 1052 | N | ALA | 173 | −6.074 | 34.121 | 5.680 | 1.00 | 0.00 | −0.650 | 9.00 | −17.40 |
| ATOM | 1053 | HN | ALA | 173 | −7.064 | 33.888 | 5.518 | 1.00 | 0.00 | 0.440 | 0.00 | 0.00 |
| ATOM | 1054 | CA | ALA | 173 | −5.733 | 35.423 | 6.247 | 1.00 | 0.00 | 0.158 | 9.40 | 4.00 |
| ATOM | 1055 | HA | ALA | 173 | −4.862 | 35.813 | 5.719 | 1.00 | 0.00 | 0.053 | 0.00 | 0.00 |
| ATOM | 1056 | CB | ALA | 173 | −6.900 | 36.401 | 6.073 | 1.00 | 0.00 | −0.159 | 16.15 | 4.00 |
| ATOM | 1057 | HB1 | ALA | 173 | −6.631 | 37.367 | 6.500 | 1.00 | 0.00 | 0.053 | 0.00 | 0.00 |
| ATOM | 1058 | HB2 | ALA | 173 | −7.118 | 36.521 | 5.012 | 1.00 | 0.00 | 0.053 | 0.00 | 0.00 |
| ATOM | 1059 | HB3 | ALA | 173 | −7.780 | 36.010 | 6.583 | 1.00 | 0.00 | 0.053 | 0.00 | 0.00 |
| ATOM | 1060 | C | ALA | 173 | −5.418 | 35.239 | 7.730 | 1.00 | 0.00 | 0.396 | 9.82 | 4.00 |
| ATOM | 1061 | O | ALA | 173 | −6.314 | 35.005 | 8.537 | 1.00 | 0.00 | −0.396 | 8.17 | −17.40 |
| ATOM | 1062 | N | VAL | 174 | −4.144 | 35.360 | 8.084 | 1.00 | 0.00 | −0.650 | 9.00 | −17.40 |
| ATOM | 1063 | HN | VAL | 174 | −3.440 | 35.574 | 7.363 | 1.00 | 0.00 | 0.440 | 0.00 | 0.00 |
| ATOM | 1064 | CA | VAL | 174 | −3.719 | 35.197 | 9.463 | 1.00 | 0.00 | 0.158 | 9.40 | 4.00 |
| ATOM | 1065 | HA | VAL | 174 | −4.308 | 34.454 | 10.001 | 1.00 | 0.00 | 0.053 | 0.00 | 0.00 |
| ATOM | 1066 | CB | VAL | 174 | −2.251 | 34.687 | 9.506 | 1.00 | 0.00 | −0.053 | 9.40 | 4.00 |
| ATOM | 1067 | HB | VAL | 174 | −1.938 | 34.530 | 10.538 | 1.00 | 0.00 | 0.053 | 0.00 | 0.00 |
| ATOM | 1068 | CG1 | VAL | 174 | −2.131 | 33.361 | 8.746 | 1.00 | 0.00 | −0.159 | 16.15 | 4.00 |
| ATOM | 1069 | HG1 | VAL | 174 | −1.098 | 33.012 | 8.782 | 1.00 | 0.00 | 0.053 | 0.00 | 0.00 |
| ATOM | 1070 | HG1 | VAL | 174 | −2.781 | 32.617 | 9.207 | 1.00 | 0.00 | 0.053 | 0.00 | 0.00 |
| ATOM | 1071 | HG1 | VAL | 174 | −2.427 | 33.508 | 7.707 | 1.00 | 0.00 | 0.053 | 0.00 | 0.00 |
| ATOM | 1072 | CG2 | VAL | 174 | −1.323 | 35.736 | 8.872 | 1.00 | 0.00 | −0.159 | 16.15 | 4.00 |
| ATOM | 1073 | HG2 | VAL | 174 | −0.294 | 35.377 | 8.902 | 1.00 | 0.00 | 0.053 | 0.00 | 0.00 |
| ATOM | 1074 | HG2 | VAL | 174 | −1.617 | 35.904 | 7.836 | 1.00 | 0.00 | 0.053 | 0.00 | 0.00 |
| ATOM | 1075 | HG2 | VAL | 174 | −1.398 | 36.671 | 9.427 | 1.00 | 0.00 | 0.053 | 0.00 | 0.00 |
| ATOM | 1076 | C | VAL | 174 | −3.829 | 36.495 | 10.276 | 1.00 | 0.00 | 0.396 | 9.82 | 4.00 |
| ATOM | 1077 | O | VAL | 174 | −3.933 | 37.586 | 9.726 | 1.00 | 0.00 | −0.396 | 8.17 | −17.40 |
| ATOM | 1078 | N | GLN | 175 | −3.838 | 36.362 | 11.599 | 1.00 | 0.00 | −0.650 | 9.00 | −17.40 |
| ATOM | 1079 | HN | GLN | 175 | −3.807 | 35.421 | 12.017 | 1.00 | 0.00 | 0.440 | 0.00 | 0.00 |
| ATOM | 1080 | CA | GLN | 175 | −3.890 | 37.528 | 12.457 | 1.00 | 0.00 | 0.158 | 9.40 | 4.00 |
| ATOM | 1081 | HA | GLN | 175 | −4.675 | 38.180 | 12.074 | 1.00 | 0.00 | 0.053 | 0.00 | 0.00 |
| ATOM | 1082 | CB | GLN | 175 | −4.268 | 37.105 | 13.879 | 1.00 | 0.00 | −0.106 | 12.77 | 4.00 |
| ATOM | 1083 | HB1 | GLN | 175 | −4.052 | 37.881 | 14.613 | 1.00 | 0.00 | 0.053 | 0.00 | 0.00 |
| ATOM | 1084 | HB2 | GLN | 175 | −3.729 | 36.214 | 14.203 | 1.00 | 0.00 | 0.053 | 0.00 | 0.00 |
| ATOM | 1085 | CG | GLN | 175 | −5.753 | 36.784 | 14.013 | 1.00 | 0.00 | −0.106 | 12.77 | 4.00 |
| ATOM | 1086 | HG1 | GLN | 175 | −5.977 | 36.337 | 14.981 | 1.00 | 0.00 | 0.053 | 0.00 | 0.00 |
| ATOM | 1087 | HG2 | GLN | 175 | −6.079 | 36.082 | 13.245 | 1.00 | 0.00 | 0.053 | 0.00 | 0.00 |
| ATOM | 1088 | CD | GLN | 175 | −6.605 | 38.035 | 13.879 | 1.00 | 0.00 | 0.396 | 9.82 | 4.00 |
| ATOM | 1089 | OE1 | GLN | 175 | −6.458 | 38.968 | 14.659 | 1.00 | 0.00 | −0.396 | 8.17 | −17.40 |
| ATOM | 1090 | NE2 | GLN | 175 | −7.493 | 38.062 | 12.884 | 1.00 | 0.00 | −0.879 | 13.25 | −17.40 |
| ATOM | 1091 | HE2 | GLN | 175 | −7.582 | 37.253 | 12.251 | 1.00 | 0.00 | 0.440 | 0.00 | 0.00 |
| ATOM | 1092 | HE2 | GLN | 175 | −8.089 | 38.890 | 12.748 | 1.00 | 0.00 | 0.440 | 0.00 | 0.00 |
| ATOM | 1093 | C | GLN | 175 | −2.520 | 38.206 | 12.393 | 1.00 | 0.00 | 0.396 | 9.82 | 4.00 |
| ATOM | 1094 | O | GLN | 175 | −1.539 | 37.589 | 11.953 | 1.00 | 0.00 | −0.396 | 8.17 | −17.40 |
| ATOM | 1095 | N | PRO | 176 | −2.428 | 39.476 | 12.828 | 1.00 | 0.00 | −0.422 | 9.00 | −17.40 |
| ATOM | 1096 | CD | PRO | 176 | −3.486 | 40.256 | 13.488 | 1.00 | 0.00 | 0.105 | 12.77 | 4.00 |
| ATOM | 1097 | HD1 | PRO | 176 | −3.544 | 40.029 | 14.552 | 1.00 | 0.00 | 0.053 | 0.00 | 0.00 |
| ATOM | 1098 | HD2 | PRO | 176 | −4.464 | 40.046 | 13.056 | 1.00 | 0.00 | 0.053 | 0.00 | 0.00 |
| ATOM | 1099 | CA | PRO | 176 | −1.171 | 40.242 | 12.802 | 1.00 | 0.00 | 0.158 | 9.40 | 4.00 |
| ATOM | 1100 | HA | PRO | 176 | −0.905 | 40.404 | 11.757 | 1.00 | 0.00 | 0.053 | 0.00 | 0.00 |
| ATOM | 1101 | CB | PRO | 176 | −1.551 | 41.569 | 13.464 | 1.00 | 0.00 | −0.106 | 12.77 | 4.00 |
| ATOM | 1102 | HB1 | PRO | 176 | −1.011 | 42.400 | 13.011 | 1.00 | 0.00 | 0.053 | 0.00 | 0.00 |
| ATOM | 1103 | HB2 | PRO | 176 | −1.315 | 41.556 | 14.528 | 1.00 | 0.00 | 0.053 | 0.00 | 0.00 |
| ATOM | 1104 | CG | PRO | 176 | −3.029 | 41.668 | 13.223 | 1.00 | 0.00 | −0.106 | 12.77 | 4.00 |
| ATOM | 1105 | HG1 | PRO | 176 | −3.244 | 41.988 | 12.203 | 1.00 | 0.00 | 0.053 | 0.00 | 0.00 |
| ATOM | 1106 | HG2 | PRO | 176 | −3.494 | 42.386 | 13.897 | 1.00 | 0.00 | 0.053 | 0.00 | 0.00 |
| ATOM | 1107 | C | PRO | 176 | 0.041 | 39.606 | 13.467 | 1.00 | 0.00 | 0.396 | 9.82 | 4.00 |
| ATOM | 1108 | O | PRO | 176 | 1.171 | 39.912 | 13.113 | 1.00 | 0.00 | −0.396 | 8.17 | −17.40 |
| ATOM | 1109 | N | ASP | 177P | −0.183 | 38.727 | 14.435 | 1.00 | 0.00 | −0.650 | 9.00 | −17.40 |
| ATOM | 1110 | HN | ASP | 177P | −1.150 | 38.495 | 14.700 | 1.00 | 0.00 | 0.440 | 0.00 | 0.00 |
| ATOM | 1111 | CA | ASP | 177P | 0.926 | 38.088 | 15.124 | 1.00 | 0.00 | 0.158 | 9.40 | 4.00 |
| ATOM | 1112 | HA | ASP | 177P | 1.773 | 38.773 | 15.121 | 1.00 | 0.00 | 0.053 | 0.00 | 0.00 |
| ATOM | 1113 | CB | ASP | 177P | 0.540 | 37.847 | 16.597 | 1.00 | 0.00 | −0.336 | 12.77 | 4.00 |
| ATOM | 1114 | HB1 | ASP | 177P | 0.315 | 38.764 | 17.141 | 1.00 | 0.00 | 0.053 | 0.00 | 0.00 |
| ATOM | 1115 | HB2 | ASP | 177P | 1.322 | 37.354 | 17.174 | 1.00 | 0.00 | 0.053 | 0.00 | 0.00 |
| ATOM | 1116 | CG | ASP | 177P | −0.706 | 36.961 | 16.764 | 1.00 | 0.00 | 0.297 | 9.82 | 4.00 |
| ATOM | 1117 | OD1 | ASP | 177P | −1.616 | 36.976 | 15.890 | 1.00 | 0.00 | −0.534 | 8.17 | −18.95 |
| ATOM | 1118 | OD2 | ASP | 177P | −0.789 | 36.255 | 17.799 | 1.00 | 0.00 | −0.534 | 8.17 | −18.95 |
| ATOM | 1119 | C | ASP | 177P | 1.382 | 36.774 | 14.479 | 1.00 | 0.00 | 0.396 | 9.82 | 4.00 |
| ATOM | 1120 | O | ASP | 177P | 2.361 | 36.173 | 14.918 | 1.00 | 0.00 | −0.396 | 8.17 | −17.40 |
| ATOM | 1121 | N | HIS | 178S | 0.713 | 36.358 | 13.405 | 1.00 | 0.00 | −0.650 | 9.00 | −17.40 |
| ATOM | 1122 | HN | HIS | 178S | −0.024 | 36.953 | 13.001 | 1.00 | 0.00 | 0.440 | 0.00 | 0.00 |
| ATOM | 1123 | CA | HIS | 178S | 1.014 | 35.062 | 12.791 | 1.00 | 0.00 | 0.158 | 9.40 | 4.00 |
| ATOM | 1124 | HA | HIS | 178S | 1.804 | 34.605 | 13.387 | 1.00 | 0.00 | 0.053 | 0.00 | 0.00 |
| ATOM | 1125 | CB | HIS | 178S | −0.218 | 34.167 | 12.927 | 1.00 | 0.00 | −0.106 | 12.77 | 4.00 |
| ATOM | 1126 | HB1 | HIS | 178S | −0.023 | 33.231 | 12.402 | 1.00 | 0.00 | 0.053 | 0.00 | 0.00 |
| ATOM | 1127 | HB2 | HIS | 178S | −1.069 | 34.683 | 12.483 | 1.00 | 0.00 | 0.053 | 0.00 | 0.00 |
| ATOM | 1128 | CG | HIS | 178S | −0.573 | 33.832 | 14.347 | 1.00 | 0.00 | −0.050 | 7.26 | 0.60 |
| ATOM | 1129 | CD2 | HIS | 178S | 0.199 | 33.688 | 15.450 | 1.00 | 0.00 | −0.177 | 10.80 | 0.60 |
| ATOM | 1130 | HD2 | HIS | 178S | 1.275 | 33.847 | 15.512 | 1.00 | 0.00 | 0.127 | 0.00 | 0.00 |
| ATOM | 1131 | ND1 | HIS | 178S | −1.861 | 33.535 | 14.740 | 1.00 | 0.00 | 0.207 | 9.25 | −17.40 |
| ATOM | 1132 | HD1 | HIS | 178S | −2.689 | 33.556 | 14.129 | 1.00 | 0.00 | 0.393 | 0.00 | 0.00 |
| ATOM | 1133 | CE1 | HIS | 178S | −1.866 | 33.219 | 16.023 | 1.00 | 0.00 | −0.227 | 10.80 | 0.60 |
| ATOM | 1134 | HE1 | HIS | 178S | −2.741 | 32.936 | 16.607 | 1.00 | 0.00 | 0.127 | 0.00 | 0.00 |
| ATOM | 1135 | NE2 | HIS | 178S | −0.629 | 33.304 | 16.476 | 1.00 | 0.00 | 0.207 | 9.25 | −17.40 |
| ATOM | 1136 | HE2 | HIS | 178S | −0.332 | 33.112 | 17.443 | 1.00 | 0.00 | 0.393 | 0.00 | 0.00 |
| ATOM | 1137 | C | HIS | 178S | 1.499 | 34.998 | 11.347 | 1.00 | 0.00 | 0.396 | 9.82 | 4.00 |
| ATOM | 1138 | O | HIS | 178S | 1.307 | 33.986 | 10.675 | 1.00 | 0.00 | −0.396 | 8.17 | −17.40 |
| ATOM | 1139 | N | ARG | 179G | 2.150 | 36.048 | 10.875 | 1.00 | 0.00 | −0.650 | 9.00 | −17.40 |
| ATOM | 1140 | HN | ARG | 179G | 2.309 | 36.862 | 11.484 | 1.00 | 0.00 | 0.440 | 0.00 | 0.00 |
| ATOM | 1141 | CA | ARG | 179G | 2.649 | 36.069 | 9.495 | 1.00 | 0.00 | 0.158 | 9.40 | 4.00 |
| ATOM | 1142 | HA | ARG | 179G | 1.838 | 36.125 | 8.768 | 1.00 | 0.00 | 0.053 | 0.00 | 0.00 |
| ATOM | 1143 | CB | ARG | 179G | 3.523 | 37.309 | 9.274 | 1.00 | 0.00 | −0.106 | 12.77 | 4.00 |
| ATOM | 1144 | HB1 | ARG | 179G | 4.558 | 36.985 | 9.168 | 1.00 | 0.00 | 0.053 | 0.00 | 0.00 |
| ATOM | 1145 | HB2 | ARG | 179G | 3.414 | 37.965 | 10.137 | 1.00 | 0.00 | 0.053 | 0.00 | 0.00 |
| ATOM | 1146 | CG | ARG | 179G | 3.154 | 38.110 | 8.029 | 1.00 | 0.00 | −0.106 | 12.77 | 4.00 |
| ATOM | 1147 | HG1 | ARG | 179G | 3.136 | 37.499 | 7.126 | 1.00 | 0.00 | 0.053 | 0.00 | 0.00 |
| ATOM | 1148 | HG2 | ARG | 179G | 3.851 | 38.923 | 7.830 | 1.00 | 0.00 | 0.053 | 0.00 | 0.00 |
| ATOM | 1149 | CD | ARG | 179G | 1.761 | 38.759 | 8.131 | 1.00 | 0.00 | 0.374 | 12.77 | 4.00 |
| ATOM | 1150 | HD1 | ARG | 179G | 0.986 | 38.053 | 8.431 | 1.00 | 0.00 | 0.053 | 0.00 | 0.00 |
| ATOM | 1151 | HD2 | ARG | 179G | 1.423 | 39.188 | 7.187 | 1.00 | 0.00 | 0.053 | 0.00 | 0.00 |
| ATOM | 1152 | NE | ARG | 179G | 1.711 | 39.851 | 9.110 | 1.00 | 0.00 | −0.819 | 9.00 | −24.67 |
| ATOM | 1153 | HE | ARG | 179G | 2.547 | 40.019 | 9.686 | 1.00 | 0.00 | 0.407 | 0.00 | 0.00 |
| ATOM | 1154 | CZ | ARG | 179G | 0.652 | 40.640 | 9.302 | 1.00 | 0.00 | 0.796 | 6.95 | 4.00 |
| ATOM | 1155 | NH1 | ARG | 179G | −0.453 | 40.463 | 8.586 | 1.00 | 0.00 | −0.746 | 9.00 | −24.67 |
| ATOM | 1156 | HH1 | ARG | 179G | −1.268 | 41.074 | 8.737 | 1.00 | 0.00 | 0.407 | 0.00 | 0.00 |
| ATOM | 1157 | HH1 | ARG | 179G | −0.494 | 39.714 | 7.879 | 1.00 | 0.00 | 0.407 | 0.00 | 0.00 |
| ATOM | 1158 | NH2 | ARG | 179G | 0.700 | 41.617 | 10.202 | 1.00 | 0.00 | −0.746 | 9.00 | −24.67 |
| ATOM | 1159 | HH2 | ARG | 179G | −0.119 | 42.223 | 10.347 | 1.00 | 0.00 | 0.407 | 0.00 | 0.00 |
| ATOM | 1160 | HH2 | ARG | 179G | 1.556 | 41.767 | 10.754 | 1.00 | 0.00 | 0.407 | 0.00 | 0.00 |
| ATOM | 1161 | C | ARG | 179G | 3.459 | 34.813 | 9.159 | 1.00 | 0.00 | 0.396 | 9.82 | 4.00 |
| ATOM | 1162 | O | ARG | 179G | 3.307 | 34.225 | 8.086 | 1.00 | 0.00 | −0.396 | 8.17 | −17.40 |
| ATOM | 1163 | N | LYS | 180S | 4.311 | 34.405 | 10.086 | 1.00 | 0.00 | −0.650 | 9.00 | −17.40 |
| ATOM | 1164 | HN | LYS | 180S | 4.378 | 34.926 | 10.971 | 1.00 | 0.00 | 0.440 | 0.00 | 0.00 |
| ATOM | 1165 | CA | LYS | 180S | 5.155 | 33.235 | 9.882 | 1.00 | 0.00 | 0.158 | 9.40 | 4.00 |
| ATOM | 1166 | HA | LYS | 180S | 5.464 | 33.216 | 8.837 | 1.00 | 0.00 | 0.053 | 0.00 | 0.00 |
| ATOM | 1167 | CB | LYS | 180S | 6.416 | 33.362 | 10.750 | 1.00 | 0.00 | −0.106 | 12.77 | 4.00 |
| ATOM | 1168 | HB1 | LYS | 180S | 7.182 | 32.632 | 10.489 | 1.00 | 0.00 | 0.053 | 0.00 | 0.00 |
| ATOM | 1169 | HB2 | LYS | 180S | 6.212 | 33.218 | 11.811 | 1.00 | 0.00 | 0.053 | 0.00 | 0.00 |
| ATOM | 1170 | CG | LYS | 180S | 7.080 | 34.732 | 10.629 | 1.00 | 0.00 | −0.106 | 12.77 | 4.00 |
| ATOM | 1171 | HG1 | LYS | 180S | 6.546 | 35.524 | 11.154 | 1.00 | 0.00 | 0.053 | 0.00 | 0.00 |
| ATOM | 1172 | HG2 | LYS | 180S | 7.169 | 35.085 | 9.602 | 1.00 | 0.00 | 0.053 | 0.00 | 0.00 |
| ATOM | 1173 | CD | LYS | 180S | 8.496 | 34.776 | 11.185 | 1.00 | 0.00 | −0.106 | 12.77 | 4.00 |
| ATOM | 1174 | HD1 | LYS | 180S | 8.950 | 35.761 | 11.087 | 1.00 | 0.00 | 0.053 | 0.00 | 0.00 |
| ATOM | 1175 | HD2 | LYS | 180S | 9.163 | 34.078 | 10.679 | 1.00 | 0.00 | 0.053 | 0.00 | 0.00 |
| ATOM | 1176 | CE | LYS | 180S | 8.550 | 34.426 | 12.655 | 1.00 | 0.00 | 0.099 | 12.77 | 4.00 |
| ATOM | 1177 | HE1 | LYS | 180S | 7.859 | 35.056 | 13.216 | 1.00 | 0.00 | 0.053 | 0.00 | 0.00 |
| ATOM | 1178 | HE2 | LYS | 180S | 9.558 | 34.579 | 13.040 | 1.00 | 0.00 | 0.053 | 0.00 | 0.00 |
| ATOM | 1179 | NZ | LYS | 180S | 8.171 | 33.002 | 12.856 | 1.00 | 0.00 | −0.045 | 13.25 | −39.20 |
| ATOM | 1180 | HZ1 | LYS | 180S | 8.210 | 32.773 | 13.859 | 1.00 | 0.00 | 0.280 | 0.00 | 0.00 |
| ATOM | 1181 | HZ2 | LYS | 180S | 7.214 | 32.848 | 12.507 | 1.00 | 0.00 | 0.280 | 0.00 | 0.00 |
| ATOM | 1182 | HZ3 | LYS | 180S | 8.825 | 32.396 | 12.340 | 1.00 | 0.00 | 0.280 | 0.00 | 0.00 |
| ATOM | 1183 | C | LYS | 180S | 4.477 | 31.896 | 10.185 | 1.00 | 0.00 | 0.396 | 9.82 | 4.00 |
| ATOM | 1184 | O | LYS | 180S | 5.102 | 30.850 | 10.068 | 1.00 | 0.00 | −0.396 | 8.17 | −17.40 |
| ATOM | 1185 | N | PHE | 181 | 3.197 | 31.904 | 10.529 | 1.00 | 0.00 | −0.650 | 9.00 | −17.40 |
| ATOM | 1186 | HN | PHE | 181 | 2.662 | 32.784 | 10.536 | 1.00 | 0.00 | 0.440 | 0.00 | 0.00 |
| ATOM | 1187 | CA | PHE | 181 | 2.558 | 30.649 | 10.896 | 1.00 | 0.00 | 0.158 | 9.40 | 4.00 |
| ATOM | 1188 | HA | PHE | 181 | 3.295 | 29.855 | 10.772 | 1.00 | 0.00 | 0.053 | 0.00 | 0.00 |
| ATOM | 1189 | CB | PHE | 181 | 2.205 | 30.680 | 12.387 | 1.00 | 0.00 | −0.106 | 12.77 | 4.00 |
| ATOM | 1190 | HB1 | PHE | 181 | 1.815 | 29.734 | 12.763 | 1.00 | 0.00 | 0.053 | 0.00 | 0.00 |
| ATOM | 1191 | HB2 | PHE | 181 | 1.445 | 31.419 | 12.640 | 1.00 | 0.00 | 0.053 | 0.00 | 0.00 |
| ATOM | 1192 | CG | PHE | 181 | 3.377 | 31.003 | 13.279 | 1.00 | 0.00 | 0.000 | 7.26 | 0.60 |
| ATOM | 1193 | CD1 | PHE | 181 | 3.570 | 32.292 | 13.762 | 1.00 | 0.00 | −0.127 | 10.80 | 0.60 |
| ATOM | 1194 | HD1 | PHE | 181 | 2.846 | 33.070 | 13.520 | 1.00 | 0.00 | 0.127 | 0.00 | 0.00 |
| ATOM | 1195 | CD2 | PHE | 181 | 4.313 | 30.023 | 13.603 | 1.00 | 0.00 | −0.127 | 10.80 | 0.60 |
| ATOM | 1196 | HD2 | PHE | 181 | 4.176 | 29.005 | 13.235 | 1.00 | 0.00 | 0.127 | 0.00 | 0.00 |
| ATOM | 1197 | CE1 | PHE | 181 | 4.675 | 32.602 | 14.550 | 1.00 | 0.00 | −0.127 | 10.80 | 0.60 |
| ATOM | 1198 | HE1 | PHE | 181 | 4.811 | 33.617 | 14.920 | 1.00 | 0.00 | 0.127 | 0.00 | 0.00 |
| ATOM | 1199 | CE2 | PHE | 181 | 5.422 | 30.327 | 14.391 | 1.00 | 0.00 | −0.127 | 10.80 | 0.60 |
| ATOM | 1200 | HE2 | PHE | 181 | 6.147 | 29.551 | 14.636 | 1.00 | 0.00 | 0.127 | 0.00 | 0.00 |
| ATOM | 1201 | CZ | PHE | 181 | 5.600 | 31.622 | 14.863 | 1.00 | 0.00 | −0.127 | 10.80 | 0.60 |
| ATOM | 1202 | HZ | PHE | 181 | 6.466 | 31.864 | 15.478 | 1.00 | 0.00 | 0.127 | 0.00 | 0.00 |
| ATOM | 1203 | C | PHE | 181 | 1.334 | 30.235 | 10.096 | 1.00 | 0.00 | 0.396 | 9.82 | 4.00 |
| ATOM | 1204 | O | PHE | 181 | 0.460 | 29.562 | 10.625 | 1.00 | 0.00 | −0.396 | 8.17 | −17.40 |
| ATOM | 1205 | N | GLN | 182 | 1.264 | 30.615 | 8.822 | 1.00 | 0.00 | −0.650 | 9.00 | −17.40 |
| ATOM | 1206 | HN | GLN | 182 | 2.021 | 31.169 | 8.399 | 1.00 | 0.00 | 0.440 | 0.00 | 0.00 |
| ATOM | 1207 | CA | GLN | 182 | 0.104 | 30.235 | 8.040 | 1.00 | 0.00 | 0.158 | 9.40 | 4.00 |
| ATOM | 1208 | HA | GLN | 182 | −0.821 | 30.603 | 8.483 | 1.00 | 0.00 | 0.053 | 0.00 | 0.00 |
| ATOM | 1209 | CB | GLN | 182 | 0.146 | 30.857 | 6.644 | 1.00 | 0.00 | −0.106 | 12.77 | 4.00 |
| ATOM | 1210 | HB1 | GLN | 182 | 0.969 | 30.447 | 6.058 | 1.00 | 0.00 | 0.053 | 0.00 | 0.00 |
| ATOM | 1211 | HB2 | GLN | 182 | 0.280 | 31.937 | 6.701 | 1.00 | 0.00 | 0.053 | 0.00 | 0.00 |
| ATOM | 1212 | CG | GLN | 182 | −1.142 | 30.587 | 5.890 | 1.00 | 0.00 | −0.106 | 12.77 | 4.00 |
| ATOM | 1213 | HG1 | GLN | 182 | −2.034 | 30.729 | 6.499 | 1.00 | 0.00 | 0.053 | 0.00 | 0.00 |
| ATOM | 1214 | HG2 | GLN | 182 | −1.210 | 29.568 | 5.507 | 1.00 | 0.00 | 0.053 | 0.00 | 0.00 |
| ATOM | 1215 | CD | GLN | 182 | −1.326 | 31.478 | 4.695 | 1.00 | 0.00 | 0.396 | 9.82 | 4.00 |
| ATOM | 1216 | OE1 | GLN | 182 | −2.264 | 31.298 | 3.924 | 1.00 | 0.00 | −0.396 | 8.17 | −17.40 |
| ATOM | 1217 | NE2 | GLN | 182 | −0.437 | 32.452 | 4.530 | 1.00 | 0.00 | −0.879 | 13.25 | −17.40 |
| ATOM | 1218 | HE2 | GLN | 182 | 0.334 | 32.564 | 5.203 | 1.00 | 0.00 | 0.440 | 0.00 | 0.00 |
| ATOM | 1219 | HE2 | GLN | 182 | −0.519 | 33.094 | 3.728 | 1.00 | 0.00 | 0.440 | 0.00 | 0.00 |
| ATOM | 1220 | C | GLN | 182 | −0.029 | 28.718 | 7.922 | 1.00 | 0.00 | 0.396 | 9.82 | 4.00 |
| ATOM | 1221 | O | GLN | 182 | −1.139 | 28.206 | 7.789 | 1.00 | 0.00 | −0.396 | 8.17 | −17.40 |
| ATOM | 1222 | N | GLY | 183 | 1.094 | 27.998 | 7.955 | 1.00 | 0.00 | −0.650 | 9.00 | −17.40 |
| ATOM | 1223 | HN | GLY | 183 | 2.006 | 28.466 | 8.045 | 1.00 | 0.00 | 0.440 | 0.00 | 0.00 |
| ATOM | 1224 | CA | GLY | 183 | 1.022 | 26.551 | 7.863 | 1.00 | 0.00 | 0.105 | 9.40 | 4.00 |
| ATOM | 1225 | HA1 | GLY | 183 | 2.007 | 26.099 | 7.978 | 1.00 | 0.00 | 0.053 | 0.00 | 0.00 |
| ATOM | 1226 | HA2 | GLY | 183 | 0.624 | 26.237 | 6.898 | 1.00 | 0.00 | 0.053 | 0.00 | 0.00 |
| ATOM | 1227 | C | GLY | 183 | 0.120 | 25.976 | 8.945 | 1.00 | 0.00 | 0.396 | 9.82 | 4.00 |
| ATOM | 1228 | O | GLY | 183 | −0.668 | 25.063 | 8.687 | 1.00 | 0.00 | −0.396 | 8.17 | −17.40 |
| ATOM | 1229 | N | TYR | 184 | 0.231 | 26.505 | 10.165 | 1.00 | 0.00 | −0.650 | 9.00 | −17.40 |
| ATOM | 1230 | HN | TYR | 184 | 0.909 | 27.262 | 10.329 | 1.00 | 0.00 | 0.440 | 0.00 | 0.00 |
| ATOM | 1231 | CA | TYR | 184 | −0.599 | 26.023 | 11.276 | 1.00 | 0.00 | 0.158 | 9.40 | 4.00 |
| ATOM | 1232 | HA | TYR | 184 | −0.501 | 24.950 | 11.442 | 1.00 | 0.00 | 0.053 | 0.00 | 0.00 |
| ATOM | 1233 | CB | TYR | 184 | −0.156 | 26.659 | 12.593 | 1.00 | 0.00 | −0.106 | 12.77 | 4.00 |
| ATOM | 1234 | HB1 | TYR | 184 | −0.796 | 26.364 | 13.424 | 1.00 | 0.00 | 0.053 | 0.00 | 0.00 |
| ATOM | 1235 | HB2 | TYR | 184 | −0.177 | 27.747 | 12.548 | 1.00 | 0.00 | 0.053 | 0.00 | 0.00 |
| ATOM | 1236 | CG | TYR | 184 | 1.251 | 26.265 | 12.965 | 1.00 | 0.00 | 0.000 | 7.26 | 0.60 |
| ATOM | 1237 | CD1 | TYR | 184 | 2.330 | 27.088 | 12.651 | 1.00 | 0.00 | −0.127 | 10.80 | 0.60 |
| ATOM | 1238 | HD1 | TYR | 184 | 2.145 | 28.054 | 12.181 | 1.00 | 0.00 | 0.127 | 0.00 | 0.00 |
| ATOM | 1239 | CE1 | TYR | 184 | 3.638 | 26.703 | 12.924 | 1.00 | 0.00 | −0.127 | 10.80 | 0.60 |
| ATOM | 1240 | HE1 | TYR | 184 | 4.468 | 27.364 | 12.675 | 1.00 | 0.00 | 0.127 | 0.00 | 0.00 |
| ATOM | 1241 | CD2 | TYR | 184 | 1.510 | 25.040 | 13.566 | 1.00 | 0.00 | −0.127 | 10.80 | 0.60 |
| ATOM | 1242 | HD2 | TYR | 184 | 0.679 | 24.385 | 13.828 | 1.00 | 0.00 | 0.127 | 0.00 | 0.00 |
| ATOM | 1243 | CE2 | TYR | 184 | 2.823 | 24.632 | 13.841 | 1.00 | 0.00 | −0.127 | 10.80 | 0.60 |
| ATOM | 1244 | HE2 | TYR | 184 | 3.012 | 23.664 | 14.306 | 1.00 | 0.00 | 0.127 | 0.00 | 0.00 |
| ATOM | 1245 | CZ | TYR | 184 | 3.879 | 25.471 | 13.516 | 1.00 | 0.00 | 0.026 | 7.26 | 0.60 |
| ATOM | 1246 | OH | TYR | 184 | 5.174 | 25.084 | 13.777 | 1.00 | 0.00 | −0.451 | 10.94 | −17.40 |
| ATOM | 1247 | HH | TYR | 184 | 5.224 | 24.663 | 14.715 | 1.00 | 0.00 | 0.424 | 0.00 | 0.00 |
| ATOM | 1248 | C | TYR | 184 | −2.093 | 26.280 | 11.056 | 1.00 | 0.00 | 0.396 | 9.82 | 4.00 |
| ATOM | 1249 | O | TYR | 184 | −2.936 | 25.521 | 11.555 | 1.00 | 0.00 | −0.396 | 8.17 | −17.40 |
| ATOM | 1250 | N | TYR | 185 | −2.419 | 27.352 | 10.330 | 1.00 | 0.00 | −0.650 | 9.00 | −17.40 |
| ATOM | 1251 | HN | TYR | 185 | −1.673 | 27.957 | 9.958 | 1.00 | 0.00 | 0.440 | 0.00 | 0.00 |
| ATOM | 1252 | CA | TYR | 185 | −3.812 | 27.679 | 10.055 | 1.00 | 0.00 | 0.158 | 9.40 | 4.00 |
| ATOM | 1253 | HA | TYR | 185 | −4.342 | 27.653 | 11.007 | 1.00 | 0.00 | 0.053 | 0.00 | 0.00 |
| ATOM | 1254 | CB | TYR | 185 | −3.942 | 29.069 | 9.406 | 1.00 | 0.00 | −0.106 | 12.77 | 4.00 |
| ATOM | 1255 | HB1 | TYR | 185 | −4.892 | 29.105 | 8.874 | 1.00 | 0.00 | 0.053 | 0.00 | 0.00 |
| ATOM | 1256 | HB2 | TYR | 185 | −3.107 | 29.199 | 8.717 | 1.00 | 0.00 | 0.053 | 0.00 | 0.00 |
| ATOM | 1257 | CG | TYR | 185 | −3.919 | 30.246 | 10.375 | 1.00 | 0.00 | 0.000 | 7.26 | 0.60 |
| ATOM | 1258 | CD1 | TYR | 185 | −2.776 | 30.545 | 11.133 | 1.00 | 0.00 | −0.127 | 10.80 | 0.60 |
| ATOM | 1259 | HD1 | TYR | 185 | −1.891 | 29.914 | 11.043 | 1.00 | 0.00 | 0.127 | 0.00 | 0.00 |
| ATOM | 1260 | CE1 | TYR | 185 | −2.755 | 31.638 | 12.000 | 1.00 | 0.00 | −0.127 | 10.80 | 0.60 |
| ATOM | 1261 | HE1 | TYR | 185 | −1.860 | 31.859 | 12.581 | 1.00 | 0.00 | 0.127 | 0.00 | 0.00 |
| ATOM | 1262 | CD2 | TYR | 185 | −5.040 | 31.072 | 10.516 | 1.00 | 0.00 | −0.127 | 10.80 | 0.60 |
| ATOM | 1263 | HD2 | TYR | 185 | −5.940 | 30.855 | 9.941 | 1.00 | 0.00 | 0.127 | 0.00 | 0.00 |
| ATOM | 1264 | CE2 | TYR | 185 | −5.025 | 32.167 | 11.378 | 1.00 | 0.00 | −0.127 | 10.80 | 0.60 |
| ATOM | 1265 | HE2 | TYR | 185 | −5.904 | 32.803 | 11.471 | 1.00 | 0.00 | 0.127 | 0.00 | 0.00 |
| ATOM | 1266 | CZ | TYR | 185 | −3.881 | 32.441 | 12.117 | 1.00 | 0.00 | 0.026 | 7.26 | 0.60 |
| ATOM | 1267 | OH | TYR | 185 | −3.887 | 33.503 | 12.996 | 1.00 | 0.00 | −0.451 | 10.94 | −17.40 |
| ATOM | 1268 | HH | TYR | 185 | −3.140 | 34.165 | 12.740 | 1.00 | 0.00 | 0.424 | 0.00 | 0.00 |
| ATOM | 1269 | C | TYR | 185 | −4.340 | 26.623 | 9.086 | 1.00 | 0.00 | 0.396 | 9.82 | 4.00 |
| ATOM | 1270 | O | TYR | 185 | −5.488 | 26.166 | 9.196 | 1.00 | 0.00 | −0.396 | 8.17 | −17.40 |
| ATOM | 1271 | N | LYS | 186S | −3.495 | 26.242 | 8.129 | 1.00 | 0.00 | −0.650 | 9.00 | −17.40 |
| ATOM | 1272 | HN | LYS | 186S | −2.554 | 26.660 | 8.089 | 1.00 | 0.00 | 0.440 | 0.00 | 0.00 |
| ATOM | 1273 | CA | LYS | 186S | −3.875 | 25.248 | 7.141 | 1.00 | 0.00 | 0.158 | 9.40 | 4.00 |
| ATOM | 1274 | HA | LYS | 186S | −4.802 | 25.533 | 6.644 | 1.00 | 0.00 | 0.053 | 0.00 | 0.00 |
| ATOM | 1275 | CB | LYS | 186S | −2.814 | 25.197 | 6.026 | 1.00 | 0.00 | −0.106 | 12.77 | 4.00 |
| ATOM | 1276 | HB1 | LYS | 186S | −3.021 | 24.388 | 5.324 | 1.00 | 0.00 | 0.053 | 0.00 | 0.00 |
| ATOM | 1277 | HB2 | LYS | 186S | −1.818 | 25.032 | 6.439 | 1.00 | 0.00 | 0.053 | 0.00 | 0.00 |
| ATOM | 1278 | CG | LYS | 186S | −2.783 | 26.514 | 5.232 | 1.00 | 0.00 | −0.106 | 12.77 | 4.00 |
| ATOM | 1279 | HG1 | LYS | 186S | −2.438 | 27.306 | 5.895 | 1.00 | 0.00 | 0.053 | 0.00 | 0.00 |
| ATOM | 1280 | HG2 | LYS | 186S | −3.791 | 26.724 | 4.876 | 1.00 | 0.00 | 0.053 | 0.00 | 0.00 |
| ATOM | 1281 | CD | LYS | 186S | −1.865 | 26.488 | 4.028 | 1.00 | 0.00 | −0.106 | 12.77 | 4.00 |
| ATOM | 1282 | HD1 | LYS | 186S | −2.089 | 25.613 | 3.417 | 1.00 | 0.00 | 0.053 | 0.00 | 0.00 |
| ATOM | 1283 | HD2 | LYS | 186S | −0.828 | 26.439 | 4.361 | 1.00 | 0.00 | 0.053 | 0.00 | 0.00 |
| ATOM | 1284 | CE | LYS | 186S | −2.074 | 27.757 | 3.197 | 1.00 | 0.00 | 0.099 | 12.77 | 4.00 |
| ATOM | 1285 | HE1 | LYS | 186S | −1.892 | 28.672 | 3.759 | 1.00 | 0.00 | 0.053 | 0.00 | 0.00 |
| ATOM | 1286 | HE2 | LYS | 186S | −3.086 | 27.851 | 2.805 | 1.00 | 0.00 | 0.053 | 0.00 | 0.00 |
| ATOM | 1287 | NZ | LYS | 186S | −1.181 | 27.834 | 2.012 | 1.00 | 0.00 | −0.045 | 13.25 | −39.20 |
| ATOM | 1288 | HZ1 | LYS | 186S | −1.368 | 28.705 | 1.496 | 1.00 | 0.00 | 0.280 | 0.00 | 0.00 |
| ATOM | 1289 | HZ2 | LYS | 186S | −0.198 | 27.822 | 2.320 | 1.00 | 0.00 | 0.280 | 0.00 | 0.00 |
| ATOM | 1290 | HZ3 | LYS | 186S | −1.356 | 27.026 | 1.396 | 1.00 | 0.00 | 0.280 | 0.00 | 0.00 |
| ATOM | 1291 | C | LYS | 186S | −4.085 | 23.889 | 7.798 | 1.00 | 0.00 | 0.396 | 9.82 | 4.00 |
| ATOM | 1292 | O | LYS | 186S | −5.029 | 23.162 | 7.441 | 1.00 | 0.00 | −0.396 | 8.17 | −17.40 |
| ATOM | 1293 | N | ILE | 187 | −3.246 | 23.555 | 8.788 | 1.00 | 0.00 | −0.650 | 9.00 | −17.40 |
| ATOM | 1294 | HN | ILE | 187 | −2.494 | 24.204 | 9.061 | 1.00 | 0.00 | 0.440 | 0.00 | 0.00 |
| ATOM | 1295 | CA | ILE | 187 | −3.389 | 22.275 | 9.484 | 1.00 | 0.00 | 0.158 | 9.40 | 4.00 |
| ATOM | 1296 | HA | ILE | 187 | −3.351 | 21.458 | 8.762 | 1.00 | 0.00 | 0.053 | 0.00 | 0.00 |
| ATOM | 1297 | CB | ILE | 187 | −2.242 | 22.027 | 10.521 | 1.00 | 0.00 | −0.053 | 9.40 | 4.00 |
| ATOM | 1298 | HB | ILE | 187 | −2.157 | 22.882 | 11.191 | 1.00 | 0.00 | 0.053 | 0.00 | 0.00 |
| ATOM | 1299 | CG2 | ILE | 187 | −2.546 | 20.781 | 11.341 | 1.00 | 0.00 | −0.159 | 16.15 | 4.00 |
| ATOM | 1300 | HG2 | ILE | 187 | −1.744 | 20.614 | 12.060 | 1.00 | 0.00 | 0.053 | 0.00 | 0.00 |
| ATOM | 1301 | HG2 | ILE | 187 | −3.488 | 20.916 | 11.872 | 1.00 | 0.00 | 0.053 | 0.00 | 0.00 |
| ATOM | 1302 | HG2 | ILE | 187 | −2.623 | 19.919 | 10.678 | 1.00 | 0.00 | 0.053 | 0.00 | 0.00 |
| ATOM | 1303 | CG1 | ILE | 187 | −0.900 | 21.861 | 9.797 | 1.00 | 0.00 | −0.106 | 12.77 | 4.00 |
| ATOM | 1304 | HG1 | ILE | 187 | −0.750 | 22.728 | 9.154 | 1.00 | 0.00 | 0.053 | 0.00 | 0.00 |
| ATOM | 1305 | HG1 | ILE | 187 | −0.943 | 20.946 | 9.205 | 1.00 | 0.00 | 0.053 | 0.00 | 0.00 |
| ATOM | 1306 | CD1 | ILE | 187 | 0.315 | 21.756 | 10.720 | 1.00 | 0.00 | −0.159 | 16.15 | 4.00 |
| ATOM | 1307 | HD1 | ILE | 187 | 1.218 | 21.641 | 10.121 | 1.00 | 0.00 | 0.053 | 0.00 | 0.00 |
| ATOM | 1308 | HD1 | ILE | 187 | 0.392 | 22.660 | 11.323 | 1.00 | 0.00 | 0.053 | 0.00 | 0.00 |
| ATOM | 1309 | HD1 | ILE | 187 | 0.201 | 20.891 | 11.374 | 1.00 | 0.00 | 0.053 | 0.00 | 0.00 |
| ATOM | 1310 | C | ILE | 187 | −4.729 | 22.236 | 10.221 | 1.00 | 0.00 | 0.396 | 9.82 | 4.00 |
| ATOM | 1311 | O | ILE | 187 | −5.483 | 21.258 | 10.116 | 1.00 | 0.00 | −0.396 | 8.17 | −17.40 |
| ATOM | 1312 | N | ALA | 188 | −5.047 | 23.298 | 10.958 | 1.00 | 0.00 | −0.650 | 9.00 | −17.40 |
| ATOM | 1313 | HN | ALA | 188 | −4.408 | 24.104 | 11.010 | 1.00 | 0.00 | 0.440 | 0.00 | 0.00 |
| ATOM | 1314 | CA | ALA | 188 | −6.308 | 23.309 | 11.690 | 1.00 | 0.00 | 0.158 | 9.40 | 4.00 |
| ATOM | 1315 | HA | ALA | 188 | −6.362 | 22.478 | 12.394 | 1.00 | 0.00 | 0.053 | 0.00 | 0.00 |
| ATOM | 1316 | CB | ALA | 188 | −6.406 | 24.571 | 12.531 | 1.00 | 0.00 | −0.159 | 16.15 | 4.00 |
| ATOM | 1317 | HB1 | ALA | 188 | −7.350 | 24.573 | 13.075 | 1.00 | 0.00 | 0.053 | 0.00 | 0.00 |
| ATOM | 1318 | HB2 | ALA | 188 | −5.578 | 24.601 | 13.240 | 1.00 | 0.00 | 0.053 | 0.00 | 0.00 |
| ATOM | 1319 | HB3 | ALA | 188 | −6.359 | 25.445 | 11.882 | 1.00 | 0.00 | 0.053 | 0.00 | 0.00 |
| ATOM | 1320 | C | ALA | 188 | −7.498 | 23.192 | 10.742 | 1.00 | 0.00 | 0.396 | 9.82 | 4.00 |
| ATOM | 1321 | O | ALA | 188 | −8.394 | 22.359 | 10.950 | 1.00 | 0.00 | −0.396 | 8.17 | −17.40 |
| ATOM | 1322 | N | ARG | 189G | −7.511 | 24.015 | 9.691 | 1.00 | 0.00 | −0.650 | 9.00 | −17.40 |
| ATOM | 1323 | HN | ARG | 189G | −6.749 | 24.697 | 9.569 | 1.00 | 0.00 | 0.440 | 0.00 | 0.00 |
| ATOM | 1324 | CA | ARG | 189G | −8.596 | 23.959 | 8.709 | 1.00 | 0.00 | 0.158 | 9.40 | 4.00 |
| ATOM | 1325 | HA | ARG | 189G | −9.537 | 24.250 | 9.173 | 1.00 | 0.00 | 0.053 | 0.00 | 0.00 |
| ATOM | 1326 | CB | ARG | 189G | −8.351 | 24.916 | 7.534 | 1.00 | 0.00 | −0.106 | 12.77 | 4.00 |
| ATOM | 1327 | HB1 | ARG | 189G | −7.335 | 24.844 | 7.144 | 1.00 | 0.00 | 0.053 | 0.00 | 0.00 |
| ATOM | 1328 | HB2 | ARG | 189G | −8.501 | 25.959 | 7.809 | 1.00 | 0.00 | 0.053 | 0.00 | 0.00 |
| ATOM | 1329 | CG | ARG | 189G | −9.290 | 24.641 | 6.348 | 1.00 | 0.00 | −0.106 | 12.77 | 4.00 |
| ATOM | 1330 | HG1 | ARG | 189G | −10.334 | 24.653 | 6.658 | 1.00 | 0.00 | 0.053 | 0.00 | 0.00 |
| ATOM | 1331 | HG2 | ARG | 189G | −9.091 | 23.666 | 5.902 | 1.00 | 0.00 | 0.053 | 0.00 | 0.00 |
| ATOM | 1332 | CD | ARG | 189G | −9.123 | 25.693 | 5.250 | 1.00 | 0.00 | 0.374 | 12.77 | 4.00 |
| ATOM | 1333 | HD1 | ARG | 189G | −9.155 | 26.705 | 5.653 | 1.00 | 0.00 | 0.053 | 0.00 | 0.00 |
| ATOM | 1334 | HD2 | ARG | 189G | −9.910 | 25.620 | 4.499 | 1.00 | 0.00 | 0.053 | 0.00 | 0.00 |
| ATOM | 1335 | NE | ARG | 189G | −7.848 | 25.548 | 4.553 | 1.00 | 0.00 | −0.819 | 9.00 | −24.67 |
| ATOM | 1336 | HE | ARG | 189G | −7.272 | 24.719 | 4.762 | 1.00 | 0.00 | 0.407 | 0.00 | 0.00 |
| ATOM | 1337 | CZ | ARG | 189G | −7.388 | 26.421 | 3.666 | 1.00 | 0.00 | 0.796 | 6.95 | 4.00 |
| ATOM | 1338 | NH1 | ARG | 189G | −8.107 | 27.500 | 3.377 | 1.00 | 0.00 | −0.746 | 9.00 | −24.67 |
| ATOM | 1339 | HH1 | ARG | 189G | −7.757 | 28.182 | 2.689 | 1.00 | 0.00 | 0.407 | 0.00 | 0.00 |
| ATOM | 1340 | HH1 | ARG | 189G | −9.014 | 27.653 | 3.840 | 1.00 | 0.00 | 0.407 | 0.00 | 0.00 |
| ATOM | 1341 | NH2 | ARG | 189G | −6.224 | 26.213 | 3.068 | 1.00 | 0.00 | −0.746 | 9.00 | −24.67 |
| ATOM | 1342 | HH2 | ARG | 189G | −5.869 | 26.892 | 2.380 | 1.00 | 0.00 | 0.407 | 0.00 | 0.00 |
| ATOM | 1343 | HH2 | ARG | 189G | −5.673 | 25.371 | 3.291 | 1.00 | 0.00 | 0.407 | 0.00 | 0.00 |
| ATOM | 1344 | C | ARG | 189G | −8.736 | 22.540 | 8.161 | 1.00 | 0.00 | 0.396 | 9.82 | 4.00 |
| ATOM | 1345 | O | ARG | 189G | −9.855 | 22.035 | 8.035 | 1.00 | 0.00 | −0.396 | 8.17 | −17.40 |
| ATOM | 1346 | N | HIS | 190S | −7.615 | 21.889 | 7.830 | 1.00 | 0.00 | −0.650 | 9.00 | −17.40 |
| ATOM | 1347 | HN | HIS | 190S | −6.700 | 22.348 | 7.940 | 1.00 | 0.00 | 0.440 | 0.00 | 0.00 |
| ATOM | 1348 | CA | HIS | 190S | −7.690 | 20.519 | 7.309 | 1.00 | 0.00 | 0.158 | 9.40 | 4.00 |
| ATOM | 1349 | HA | HIS | 190S | −8.325 | 20.459 | 6.425 | 1.00 | 0.00 | 0.053 | 0.00 | 0.00 |
| ATOM | 1350 | CB | HIS | 190S | −6.326 | 19.994 | 6.848 | 1.00 | 0.00 | −0.106 | 12.77 | 4.00 |
| ATOM | 1351 | HB1 | HIS | 190S | −5.620 | 19.848 | 7.666 | 1.00 | 0.00 | 0.053 | 0.00 | 0.00 |
| ATOM | 1352 | HB2 | HIS | 190S | −5.821 | 20.659 | 6.147 | 1.00 | 0.00 | 0.053 | 0.00 | 0.00 |
| ATOM | 1353 | CG | HIS | 190S | −6.404 | 18.668 | 6.150 | 1.00 | 0.00 | −0.050 | 7.26 | 0.60 |
| ATOM | 1354 | CD2 | HIS | 190S | −7.456 | 18.007 | 5.602 | 1.00 | 0.00 | −0.177 | 10.80 | 0.60 |
| ATOM | 1355 | HD2 | HIS | 190S | −8.496 | 18.331 | 5.607 | 1.00 | 0.00 | 0.127 | 0.00 | 0.00 |
| ATOM | 1356 | ND1 | HIS | 190S | −5.295 | 17.887 | 5.903 | 1.00 | 0.00 | 0.207 | 9.25 | −17.40 |
| ATOM | 1357 | HD1 | HIS | 190S | −4.332 | 18.112 | 6.193 | 1.00 | 0.00 | 0.393 | 0.00 | 0.00 |
| ATOM | 1358 | CE1 | HIS | 190S | −5.657 | 16.804 | 5.236 | 1.00 | 0.00 | −0.227 | 10.80 | 0.60 |
| ATOM | 1359 | HE1 | HIS | 190S | −4.991 | 16.006 | 4.905 | 1.00 | 0.00 | 0.127 | 0.00 | 0.00 |
| ATOM | 1360 | NE2 | HIS | 190S | −6.965 | 16.853 | 5.039 | 1.00 | 0.00 | 0.207 | 9.25 | −17.40 |
| ATOM | 1361 | HE2 | HIS | 190S | −7.523 | 16.142 | 4.544 | 1.00 | 0.00 | 0.393 | 0.00 | 0.00 |
| ATOM | 1362 | C | HIS | 190S | −8.264 | 19.553 | 8.345 | 1.00 | 0.00 | 0.396 | 9.82 | 4.00 |
| ATOM | 1363 | O | HIS | 190S | −9.140 | 18.744 | 8.017 | 1.00 | 0.00 | −0.396 | 8.17 | −17.40 |
| ATOM | 1364 | N | TYR | 191 | −7.771 | 19.609 | 9.584 | 1.00 | 0.00 | −0.650 | 9.00 | −17.40 |
| ATOM | 1365 | HN | TYR | 191 | −7.011 | 20.269 | 9.803 | 1.00 | 0.00 | 0.440 | 0.00 | 0.00 |
| ATOM | 1366 | CA | TYR | 191 | −8.303 | 18.736 | 10.635 | 1.00 | 0.00 | 0.158 | 9.40 | 4.00 |
| ATOM | 1367 | HA | TYR | 191 | −8.098 | 17.688 | 10.411 | 1.00 | 0.00 | 0.053 | 0.00 | 0.00 |
| ATOM | 1368 | CB | TYR | 191 | −7.663 | 19.042 | 11.995 | 1.00 | 0.00 | −0.106 | 12.77 | 4.00 |
| ATOM | 1369 | HB1 | TYR | 191 | −8.346 | 18.891 | 12.830 | 1.00 | 0.00 | 0.053 | 0.00 | 0.00 |
| ATOM | 1370 | HB2 | TYR | 191 | −7.314 | 20.071 | 12.079 | 1.00 | 0.00 | 0.053 | 0.00 | 0.00 |
| ATOM | 1371 | CG | TYR | 191 | −6.469 | 18.183 | 12.286 | 1.00 | 0.00 | 0.000 | 7.26 | 0.60 |
| ATOM | 1372 | CD1 | TYR | 191 | −5.311 | 18.285 | 11.512 | 1.00 | 0.00 | −0.127 | 10.80 | 0.60 |
| ATOM | 1373 | HD1 | TYR | 191 | −5.275 | 19.001 | 10.691 | 1.00 | 0.00 | 0.127 | 0.00 | 0.00 |
| ATOM | 1374 | CE1 | TYR | 191 | −4.206 | 17.490 | 11.771 | 1.00 | 0.00 | −0.127 | 10.80 | 0.60 |
| ATOM | 1375 | HE1 | TYR | 191 | −3.308 | 17.578 | 11.158 | 1.00 | 0.00 | 0.127 | 0.00 | 0.00 |
| ATOM | 1376 | CD2 | TYR | 191 | −6.495 | 17.261 | 13.335 | 1.00 | 0.00 | −0.127 | 10.80 | 0.60 |
| ATOM | 1377 | HD2 | TYR | 191 | −7.392 | 17.169 | 13.947 | 1.00 | 0.00 | 0.127 | 0.00 | 0.00 |
| ATOM | 1378 | CE2 | TYR | 191 | −5.396 | 16.460 | 13.609 | 1.00 | 0.00 | −0.127 | 10.80 | 0.60 |
| ATOM | 1379 | HE2 | TYR | 191 | −5.427 | 15.746 | 14.432 | 1.00 | 0.00 | 0.127 | 0.00 | 0.00 |
| ATOM | 1380 | CZ | TYR | 191 | −4.254 | 16.574 | 12.825 | 1.00 | 0.00 | 0.026 | 7.26 | 0.60 |
| ATOM | 1381 | OH | TYR | 191 | −3.164 | 15.771 | 13.087 | 1.00 | 0.00 | −0.451 | 10.94 | −17.40 |
| ATOM | 1382 | HH | TYR | 191 | −3.457 | 14.970 | 13.665 | 1.00 | 0.00 | 0.424 | 0.00 | 0.00 |
| ATOM | 1383 | C | TYR | 191 | −9.809 | 18.902 | 10.768 | 1.00 | 0.00 | 0.396 | 9.82 | 4.00 |
| ATOM | 1384 | O | TYR | 191 | −10.550 | 17.916 | 10.865 | 1.00 | 0.00 | −0.396 | 8.17 | −17.40 |
| ATOM | 1385 | N | ARG | 192G | −10.274 | 20.149 | 10.767 | 1.00 | 0.00 | −0.650 | 9.00 | −17.40 |
| ATOM | 1386 | HN | ARG | 192G | −9.627 | 20.944 | 10.670 | 1.00 | 0.00 | 0.440 | 0.00 | 0.00 |
| ATOM | 1387 | CA | ARG | 192G | −11.705 | 20.378 | 10.904 | 1.00 | 0.00 | 0.158 | 9.40 | 4.00 |
| ATOM | 1388 | HA | ARG | 192G | −12.066 | 19.985 | 11.854 | 1.00 | 0.00 | 0.053 | 0.00 | 0.00 |
| ATOM | 1389 | CB | ARG | 192G | −12.019 | 21.869 | 10.893 | 1.00 | 0.00 | −0.106 | 12.77 | 4.00 |
| ATOM | 1390 | HB1 | ARG | 192G | −11.918 | 22.316 | 9.904 | 1.00 | 0.00 | 0.053 | 0.00 | 0.00 |
| ATOM | 1391 | HB2 | ARG | 192G | −11.365 | 22.445 | 11.548 | 1.00 | 0.00 | 0.053 | 0.00 | 0.00 |
| ATOM | 1392 | CG | ARG | 192G | −13.433 | 22.178 | 11.344 | 1.00 | 0.00 | −0.106 | 12.77 | 4.00 |
| ATOM | 1393 | HG1 | ARG | 192G | −13.591 | 21.897 | 12.385 | 1.00 | 0.00 | 0.053 | 0.00 | 0.00 |
| ATOM | 1394 | HG2 | ARG | 192G | −14.170 | 21.640 | 10.747 | 1.00 | 0.00 | 0.053 | 0.00 | 0.00 |
| ATOM | 1395 | CD | ARG | 192G | −13.746 | 23.656 | 11.223 | 1.00 | 0.00 | 0.374 | 12.77 | 4.00 |
| ATOM | 1396 | HD1 | ARG | 192G | −14.547 | 23.844 | 10.509 | 1.00 | 0.00 | 0.053 | 0.00 | 0.00 |
| ATOM | 1397 | HD2 | ARG | 192G | −12.881 | 24.228 | 10.888 | 1.00 | 0.00 | 0.053 | 0.00 | 0.00 |
| ATOM | 1398 | NE | ARG | 192G | −14.169 | 24.231 | 12.501 | 1.00 | 0.00 | −0.819 | 9.00 | −24.67 |
| ATOM | 1399 | HE | ARG | 192G | −13.914 | 23.729 | 13.364 | 1.00 | 0.00 | 0.407 | 0.00 | 0.00 |
| ATOM | 1400 | CZ | ARG | 192G | −14.863 | 25.362 | 12.618 | 1.00 | 0.00 | 0.796 | 6.95 | 4.00 |
| ATOM | 1401 | NH1 | ARG | 192G | −15.220 | 26.034 | 11.527 | 1.00 | 0.00 | −0.746 | 9.00 | −24.67 |
| ATOM | 1402 | HH1 | ARG | 192G | −15.756 | 26.908 | 11.613 | 1.00 | 0.00 | 0.407 | 0.00 | 0.00 |
| ATOM | 1403 | HH1 | ARG | 192G | −14.959 | 25.679 | 10.595 | 1.00 | 0.00 | 0.407 | 0.00 | 0.00 |
| ATOM | 1404 | NH2 | ARG | 192G | −15.186 | 25.829 | 13.823 | 1.00 | 0.00 | −0.746 | 9.00 | −24.67 |
| ATOM | 1405 | HH2 | ARG | 192G | −15.722 | 26.703 | 13.909 | 1.00 | 0.00 | 0.407 | 0.00 | 0.00 |
| ATOM | 1406 | HH2 | ARG | 192G | −14.899 | 25.315 | 14.668 | 1.00 | 0.00 | 0.407 | 0.00 | 0.00 |
| ATOM | 1407 | C | ARG | 192G | −12.469 | 19.687 | 9.777 | 1.00 | 0.00 | 0.396 | 9.82 | 4.00 |
| ATOM | 1408 | O | ARG | 192G | −13.436 | 18.976 | 10.018 | 1.00 | 0.00 | −0.396 | 8.17 | −17.40 |
| ATOM | 1409 | N | TRP | 193 | −12.027 | 19.887 | 8.541 | 1.00 | 0.00 | −0.650 | 9.00 | −17.40 |
| ATOM | 1410 | HN | TRP | 193 | −11.204 | 20.485 | 8.383 | 1.00 | 0.00 | 0.440 | 0.00 | 0.00 |
| ATOM | 1411 | CA | TRP | 193 | −12.695 | 19.268 | 7.411 | 1.00 | 0.00 | 0.158 | 9.40 | 4.00 |
| ATOM | 1412 | HA | TRP | 193 | −13.758 | 19.501 | 7.369 | 1.00 | 0.00 | 0.053 | 0.00 | 0.00 |
| ATOM | 1413 | CB | TRP | 193 | −12.122 | 19.792 | 6.078 | 1.00 | 0.00 | −0.106 | 12.77 | 4.00 |
| ATOM | 1414 | HB1 | TRP | 193 | −11.075 | 19.519 | 5.944 | 1.00 | 0.00 | 0.053 | 0.00 | 0.00 |
| ATOM | 1415 | HB2 | TRP | 193 | −12.172 | 20.878 | 6.006 | 1.00 | 0.00 | 0.053 | 0.00 | 0.00 |
| ATOM | 1416 | CG | TRP | 193 | −12.871 | 19.242 | 4.874 | 1.00 | 0.00 | 0.000 | 7.26 | 0.60 |
| ATOM | 1417 | CD2 | TRP | 193 | −12.535 | 18.074 | 4.114 | 1.00 | 0.00 | 0.000 | 6.80 | 0.60 |
| ATOM | 1418 | CE2 | TRP | 193 | −13.549 | 17.898 | 3.134 | 1.00 | 0.00 | −0.050 | 6.80 | 0.60 |
| ATOM | 1419 | CD1 | TRP | 193 | −14.043 | 19.714 | 4.339 | 1.00 | 0.00 | −0.177 | 10.80 | 0.60 |
| ATOM | 1420 | HD1 | TRP | 193 | −14.575 | 20.598 | 4.690 | 1.00 | 0.00 | 0.127 | 0.00 | 0.00 |
| ATOM | 1421 | NE1 | TRP | 193 | −14.453 | 18.913 | 3.295 | 1.00 | 0.00 | −0.292 | 9.00 | −17.40 |
| ATOM | 1422 | HE1 | TRP | 193 | −15.301 | 19.055 | 2.728 | 1.00 | 0.00 | 0.393 | 0.00 | 0.00 |
| ATOM | 1423 | CE3 | TRP | 193 | −11.479 | 17.156 | 4.166 | 1.00 | 0.00 | −0.127 | 10.80 | 0.60 |
| ATOM | 1424 | HE3 | TRP | 193 | −10.688 | 17.259 | 4.909 | 1.00 | 0.00 | 0.127 | 0.00 | 0.00 |
| ATOM | 1425 | CZ2 | TRP | 193 | −13.532 | 16.841 | 2.211 | 1.00 | 0.00 | −0.127 | 10.80 | 0.60 |
| ATOM | 1426 | HZ2 | TRP | 193 | −14.321 | 16.726 | 1.468 | 1.00 | 0.00 | 0.127 | 0.00 | 0.00 |
| ATOM | 1427 | CZ3 | TRP | 193 | −11.459 | 16.101 | 3.243 | 1.00 | 0.00 | −0.127 | 10.80 | 0.60 |
| ATOM | 1428 | HZ3 | TRP | 193 | −10.641 | 15.380 | 3.269 | 1.00 | 0.00 | 0.127 | 0.00 | 0.00 |
| ATOM | 1429 | CH2 | TRP | 193 | −12.483 | 15.956 | 2.278 | 1.00 | 0.00 | −0.127 | 10.80 | 0.60 |
| ATOM | 1430 | HH2 | TRP | 193 | −12.439 | 15.125 | 1.573 | 1.00 | 0.00 | 0.127 | 0.00 | 0.00 |
| ATOM | 1431 | C | TRP | 193 | −12.600 | 17.739 | 7.430 | 1.00 | 0.00 | 0.396 | 9.82 | 4.00 |
| ATOM | 1432 | O | TRP | 193 | −13.615 | 17.049 | 7.230 | 1.00 | 0.00 | −0.396 | 8.17 | −17.40 |
| ATOM | 1433 | N | ALA | 194 | −11.398 | 17.208 | 7.681 | 1.00 | 0.00 | −0.650 | 9.00 | −17.40 |
| ATOM | 1434 | HN | ALA | 194 | −10.601 | 17.832 | 7.872 | 1.00 | 0.00 | 0.440 | 0.00 | 0.00 |
| ATOM | 1435 | CA | ALA | 194 | −11.190 | 15.755 | 7.690 | 1.00 | 0.00 | 0.158 | 9.40 | 4.00 |
| ATOM | 1436 | HA | ALA | 194 | −11.494 | 15.299 | 6.747 | 1.00 | 0.00 | 0.053 | 0.00 | 0.00 |
| ATOM | 1437 | CB | ALA | 194 | −9.701 | 15.422 | 7.849 | 1.00 | 0.00 | −0.159 | 16.15 | 4.00 |
| ATOM | 1438 | HB1 | ALA | 194 | −9.568 | 14.340 | 7.853 | 1.00 | 0.00 | 0.053 | 0.00 | 0.00 |
| ATOM | 1439 | HB2 | ALA | 194 | −9.141 | 15.852 | 7.019 | 1.00 | 0.00 | 0.053 | 0.00 | 0.00 |
| ATOM | 1440 | HB3 | ALA | 194 | −9.333 | 15.836 | 8.788 | 1.00 | 0.00 | 0.053 | 0.00 | 0.00 |
| ATOM | 1441 | C | ALA | 194 | −11.986 | 15.083 | 8.790 | 1.00 | 0.00 | 0.396 | 9.82 | 4.00 |
| ATOM | 1442 | O | ALA | 194 | −12.639 | 14.068 | 8.552 | 1.00 | 0.00 | −0.396 | 8.17 | −17.40 |
| ATOM | 1443 | N | LEU | 195 | −11.936 | 15.638 | 9.998 | 1.00 | 0.00 | −0.650 | 9.00 | −17.40 |
| ATOM | 1444 | HN | LEU | 195 | −11.369 | 16.482 | 10.162 | 1.00 | 0.00 | 0.440 | 0.00 | 0.00 |
| ATOM | 1445 | CA | LEU | 195 | −12.695 | 15.034 | 11.086 | 1.00 | 0.00 | 0.158 | 9.40 | 4.00 |
| ATOM | 1446 | HA | LEU | 195 | −12.448 | 13.978 | 11.203 | 1.00 | 0.00 | 0.053 | 0.00 | 0.00 |
| ATOM | 1447 | CB | LEU | 195 | −12.295 | 15.669 | 12.422 | 1.00 | 0.00 | −0.106 | 12.77 | 4.00 |
| ATOM | 1448 | HB1 | LEU | 195 | −12.965 | 15.377 | 13.230 | 1.00 | 0.00 | 0.053 | 0.00 | 0.00 |
| ATOM | 1449 | HB2 | LEU | 195 | −12.310 | 16.758 | 12.378 | 1.00 | 0.00 | 0.053 | 0.00 | 0.00 |
| ATOM | 1450 | CG | LEU | 195 | −10.874 | 15.261 | 12.853 | 1.00 | 0.00 | −0.053 | 9.40 | 4.00 |
| ATOM | 1451 | HG | LEU | 195 | −10.173 | 15.397 | 12.029 | 1.00 | 0.00 | 0.053 | 0.00 | 0.00 |
| ATOM | 1452 | CD1 | LEU | 195 | −10.420 | 16.097 | 14.007 | 1.00 | 0.00 | −0.159 | 16.15 | 4.00 |
| ATOM | 1453 | HD1 | LEU | 195 | −9.414 | 15.796 | 14.300 | 1.00 | 0.00 | 0.053 | 0.00 | 0.00 |
| ATOM | 1454 | HD1 | LEU | 195 | −10.414 | 17.147 | 13.715 | 1.00 | 0.00 | 0.053 | 0.00 | 0.00 |
| ATOM | 1455 | HD1 | LEU | 195 | −11.099 | 15.957 | 14.847 | 1.00 | 0.00 | 0.053 | 0.00 | 0.00 |
| ATOM | 1456 | CD2 | LEU | 195 | −10.849 | 13.777 | 13.218 | 1.00 | 0.00 | −0.159 | 16.15 | 4.00 |
| ATOM | 1457 | HD2 | LEU | 195 | −9.841 | 13.494 | 13.522 | 1.00 | 0.00 | 0.053 | 0.00 | 0.00 |
| ATOM | 1458 | HD2 | LEU | 195 | −11.541 | 13.592 | 14.039 | 1.00 | 0.00 | 0.053 | 0.00 | 0.00 |
| ATOM | 1459 | HD2 | LEU | 195 | −11.146 | 13.184 | 12.353 | 1.00 | 0.00 | 0.053 | 0.00 | 0.00 |
| ATOM | 1460 | C | LEU | 195 | −14.208 | 15.131 | 10.833 | 1.00 | 0.00 | 0.396 | 9.82 | 4.00 |
| ATOM | 1461 | O | LEU | 195 | −14.958 | 14.225 | 11.191 | 1.00 | 0.00 | −0.396 | 8.17 | −17.40 |
| ATOM | 1462 | N | GLY | 196 | −14.644 | 16.219 | 10.206 | 1.00 | 0.00 | −0.650 | 9.00 | −17.40 |
| ATOM | 1463 | HN | GLY | 196 | −13.975 | 16.955 | 9.939 | 1.00 | 0.00 | 0.440 | 0.00 | 0.00 |
| ATOM | 1464 | CA | GLY | 196 | −16.055 | 16.381 | 9.893 | 1.00 | 0.00 | 0.105 | 9.40 | 4.00 |
| ATOM | 1465 | HA1 | GLY | 196 | −16.226 | 17.375 | 9.481 | 1.00 | 0.00 | 0.053 | 0.00 | 0.00 |
| ATOM | 1466 | HA2 | GLY | 196 | −16.647 | 16.259 | 10.800 | 1.00 | 0.00 | 0.053 | 0.00 | 0.00 |
| ATOM | 1467 | C | GLY | 196 | −16.475 | 15.334 | 8.873 | 1.00 | 0.00 | 0.396 | 9.82 | 4.00 |
| ATOM | 1468 | O | GLY | 196 | −17.610 | 14.865 | 8.884 | 1.00 | 0.00 | −0.396 | 8.17 | −17.40 |
| ATOM | 1469 | N | GLN | 197 | −15.571 | 14.965 | 7.972 | 1.00 | 0.00 | −0.650 | 9.00 | −17.40 |
| ATOM | 1470 | HN | GLN | 197 | −14.638 | 15.403 | 7.973 | 1.00 | 0.00 | 0.440 | 0.00 | 0.00 |
| ATOM | 1471 | CA | GLN | 197 | −15.890 | 13.943 | 6.981 | 1.00 | 0.00 | 0.158 | 9.40 | 4.00 |
| ATOM | 1472 | HA | GLN | 197 | −16.796 | 14.223 | 6.443 | 1.00 | 0.00 | 0.053 | 0.00 | 0.00 |
| ATOM | 1473 | CB | GLN | 197 | −14.749 | 13.812 | 5.955 | 1.00 | 0.00 | −0.106 | 12.77 | 4.00 |
| ATOM | 1474 | HB1 | GLN | 197 | −14.887 | 12.878 | 5.410 | 1.00 | 0.00 | 0.053 | 0.00 | 0.00 |
| ATOM | 1475 | HB2 | GLN | 197 | −13.803 | 13.806 | 6.497 | 1.00 | 0.00 | 0.053 | 0.00 | 0.00 |
| ATOM | 1476 | CG | GLN | 197 | −14.681 | 14.942 | 4.922 | 1.00 | 0.00 | −0.106 | 12.77 | 4.00 |
| ATOM | 1477 | HG1 | GLN | 197 | −13.827 | 14.852 | 4.250 | 1.00 | 0.00 | 0.053 | 0.00 | 0.00 |
| ATOM | 1478 | HG2 | GLN | 197 | −14.598 | 15.928 | 5.377 | 1.00 | 0.00 | 0.053 | 0.00 | 0.00 |
| ATOM | 1479 | CD | GLN | 197 | −15.913 | 15.000 | 4.023 | 1.00 | 0.00 | 0.396 | 9.82 | 4.00 |
| ATOM | 1480 | OE1 | GLN | 197 | −16.246 | 14.024 | 3.350 | 1.00 | 0.00 | −0.396 | 8.17 | −17.40 |
| ATOM | 1481 | NE2 | GLN | 197 | −16.594 | 16.147 | 4.011 | 1.00 | 0.00 | −0.879 | 13.25 | −17.40 |
| ATOM | 1482 | HE2 | GLN | 197 | −16.279 | 16.938 | 4.590 | 1.00 | 0.00 | 0.440 | 0.00 | 0.00 |
| ATOM | 1483 | HE2 | GLN | 197 | −17.434 | 16.241 | 3.422 | 1.00 | 0.00 | 0.440 | 0.00 | 0.00 |
| ATOM | 1484 | C | GLN | 197 | −16.113 | 12.594 | 7.683 | 1.00 | 0.00 | 0.396 | 9.82 | 4.00 |
| ATOM | 1485 | O | GLN | 197 | −17.068 | 11.870 | 7.387 | 1.00 | 0.00 | −0.396 | 8.17 | −17.40 |
| ATOM | 1486 | N | ILE | 198 | −15.213 | 12.269 | 8.607 | 1.00 | 0.00 | −0.650 | 9.00 | −17.40 |
| ATOM | 1487 | HN | ILE | 198 | −14.443 | 12.926 | 8.797 | 1.00 | 0.00 | 0.440 | 0.00 | 0.00 |
| ATOM | 1488 | CA | ILE | 198 | −15.267 | 11.022 | 9.363 | 1.00 | 0.00 | 0.158 | 9.40 | 4.00 |
| ATOM | 1489 | HA | ILE | 198 | −15.221 | 10.136 | 8.729 | 1.00 | 0.00 | 0.053 | 0.00 | 0.00 |
| ATOM | 1490 | CB | ILE | 198 | −14.068 | 10.937 | 10.370 | 1.00 | 0.00 | −0.053 | 9.40 | 4.00 |
| ATOM | 1491 | HB | ILE | 198 | −13.945 | 11.893 | 10.878 | 1.00 | 0.00 | 0.053 | 0.00 | 0.00 |
| ATOM | 1492 | CG2 | ILE | 198 | −14.338 | 9.861 | 11.395 | 1.00 | 0.00 | −0.159 | 16.15 | 4.00 |
| ATOM | 1493 | HG2 | ILE | 198 | −13.502 | 9.805 | 12.092 | 1.00 | 0.00 | 0.053 | 0.00 | 0.00 |
| ATOM | 1494 | HG2 | ILE | 198 | −15.251 | 10.099 | 11.940 | 1.00 | 0.00 | 0.053 | 0.00 | 0.00 |
| ATOM | 1495 | HG2 | ILE | 198 | −14.455 | 8.901 | 10.892 | 1.00 | 0.00 | 0.053 | 0.00 | 0.00 |
| ATOM | 1496 | CG1 | ILE | 198 | −12.745 | 10.685 | 9.639 | 1.00 | 0.00 | −0.106 | 12.77 | 4.00 |
| ATOM | 1497 | HG1 | ILE | 198 | −11.931 | 10.806 | 10.354 | 1.00 | 0.00 | 0.053 | 0.00 | 0.00 |
| ATOM | 1498 | HG1 | ILE | 198 | −12.658 | 11.410 | 8.830 | 1.00 | 0.00 | 0.053 | 0.00 | 0.00 |
| ATOM | 1499 | CD1 | ILE | 198 | −12.613 | 9.297 | 9.026 | 1.00 | 0.00 | −0.159 | 16.15 | 4.00 |
| ATOM | 1500 | HD1 | ILE | 198 | −11.646 | 9.208 | 8.530 | 1.00 | 0.00 | 0.053 | 0.00 | 0.00 |
| ATOM | 1501 | HD1 | ILE | 198 | −12.688 | 8.544 | 9.810 | 1.00 | 0.00 | 0.053 | 0.00 | 0.00 |
| ATOM | 1502 | HD1 | ILE | 198 | −13.409 | 9.143 | 8.298 | 1.00 | 0.00 | 0.053 | 0.00 | 0.00 |
| ATOM | 1503 | C | ILE | 198 | −16.549 | 10.875 | 10.187 | 1.00 | 0.00 | 0.396 | 9.82 | 4.00 |
| ATOM | 1504 | O | ILE | 198 | −17.201 | 9.817 | 10.190 | 1.00 | 0.00 | −0.396 | 8.17 | −17.40 |
| ATOM | 1505 | N | PHE | 199 | −16.901 | 11.936 | 10.903 | 1.00 | 0.00 | −0.650 | 9.00 | −17.40 |
| ATOM | 1506 | HN | PHE | 199 | −16.348 | 12.802 | 10.836 | 1.00 | 0.00 | 0.440 | 0.00 | 0.00 |
| ATOM | 1507 | CA | PHE | 199 | −18.062 | 11.886 | 11.783 | 1.00 | 0.00 | 0.158 | 9.40 | 4.00 |
| ATOM | 1508 | HA | PHE | 199 | −18.210 | 10.844 | 12.066 | 1.00 | 0.00 | 0.053 | 0.00 | 0.00 |
| ATOM | 1509 | CB | PHE | 199 | −17.737 | 12.622 | 13.089 | 1.00 | 0.00 | −0.106 | 12.77 | 4.00 |
| ATOM | 1510 | HB1 | PHE | 199 | −18.551 | 12.611 | 13.813 | 1.00 | 0.00 | 0.053 | 0.00 | 0.00 |
| ATOM | 1511 | HB2 | PHE | 199 | −17.499 | 13.676 | 12.951 | 1.00 | 0.00 | 0.053 | 0.00 | 0.00 |
| ATOM | 1512 | CG | PHE | 199 | −16.550 | 12.051 | 13.831 | 1.00 | 0.00 | 0.000 | 7.26 | 0.60 |
| ATOM | 1513 | CD1 | PHE | 199 | −15.377 | 12.777 | 13.949 | 1.00 | 0.00 | −0.127 | 10.80 | 0.60 |
| ATOM | 1514 | HD1 | PHE | 199 | −15.307 | 13.760 | 13.485 | 1.00 | 0.00 | 0.127 | 0.00 | 0.00 |
| ATOM | 1515 | CD2 | PHE | 199 | −16.614 | 10.787 | 14.426 | 1.00 | 0.00 | −0.127 | 10.80 | 0.60 |
| ATOM | 1516 | HD2 | PHE | 199 | −17.528 | 10.199 | 14.341 | 1.00 | 0.00 | 0.127 | 0.00 | 0.00 |
| ATOM | 1517 | CE1 | PHE | 199 | −14.280 | 12.272 | 14.652 | 1.00 | 0.00 | −0.127 | 10.80 | 0.60 |
| ATOM | 1518 | HE1 | PHE | 199 | −13.368 | 12.863 | 14.735 | 1.00 | 0.00 | 0.127 | 0.00 | 0.00 |
| ATOM | 1519 | CE2 | PHE | 199 | −15.514 | 10.262 | 15.134 | 1.00 | 0.00 | −0.127 | 10.80 | 0.60 |
| ATOM | 1520 | HE2 | PHE | 199 | −15.576 | 9.273 | 15.589 | 1.00 | 0.00 | 0.127 | 0.00 | 0.00 |
| ATOM | 1521 | CZ | PHE | 199 | −14.349 | 11.011 | 15.248 | 1.00 | 0.00 | −0.127 | 10.80 | 0.60 |
| ATOM | 1522 | HZ | PHE | 199 | −13.495 | 10.616 | 15.799 | 1.00 | 0.00 | 0.127 | 0.00 | 0.00 |
| ATOM | 1523 | C | PHE | 199 | −19.399 | 12.375 | 11.236 | 1.00 | 0.00 | 0.396 | 9.82 | 4.00 |
| ATOM | 1524 | O | PHE | 199 | −20.447 | 12.015 | 11.776 | 1.00 | 0.00 | −0.396 | 8.17 | −17.40 |
| ATOM | 1525 | N | HIS | 200S | −19.381 | 13.177 | 10.173 | 1.00 | 0.00 | −0.650 | 9.00 | −17.40 |
| ATOM | 1526 | HN | HIS | 200S | −18.480 | 13.443 | 9.751 | 1.00 | 0.00 | 0.440 | 0.00 | 0.00 |
| ATOM | 1527 | CA | HIS | 200S | −20.627 | 13.683 | 9.598 | 1.00 | 0.00 | 0.158 | 9.40 | 4.00 |
| ATOM | 1528 | HA | HIS | 200S | −21.476 | 13.558 | 10.269 | 1.00 | 0.00 | 0.053 | 0.00 | 0.00 |
| ATOM | 1529 | CB | HIS | 200S | −20.539 | 15.186 | 9.317 | 1.00 | 0.00 | −0.106 | 12.77 | 4.00 |
| ATOM | 1530 | HB1 | HIS | 200S | −21.467 | 15.501 | 8.840 | 1.00 | 0.00 | 0.053 | 0.00 | 0.00 |
| ATOM | 1531 | HB2 | HIS | 200S | −19.690 | 15.363 | 8.656 | 1.00 | 0.00 | 0.053 | 0.00 | 0.00 |
| ATOM | 1532 | CG | HIS | 200S | −20.347 | 16.023 | 10.542 | 1.00 | 0.00 | −0.050 | 7.26 | 0.60 |
| ATOM | 1533 | CD2 | HIS | 200S | −19.545 | 17.089 | 10.775 | 1.00 | 0.00 | −0.177 | 10.80 | 0.60 |
| ATOM | 1534 | HD2 | HIS | 200S | −18.861 | 17.552 | 10.064 | 1.00 | 0.00 | 0.127 | 0.00 | 0.00 |
| ATOM | 1535 | ND1 | HIS | 200S | −21.034 | 15.792 | 11.718 | 1.00 | 0.00 | 0.207 | 9.25 | −17.40 |
| ATOM | 1536 | HD1 | HIS | 200S | −21.728 | 15.046 | 11.865 | 1.00 | 0.00 | 0.393 | 0.00 | 0.00 |
| ATOM | 1537 | CE1 | HIS | 200S | −20.659 | 16.679 | 12.624 | 1.00 | 0.00 | −0.227 | 10.80 | 0.60 |
| ATOM | 1538 | HE1 | HIS | 200S | −21.030 | 16.742 | 13.646 | 1.00 | 0.00 | 0.127 | 0.00 | 0.00 |
| ATOM | 1539 | NE2 | HIS | 200S | −19.756 | 17.477 | 12.078 | 1.00 | 0.00 | 0.207 | 9.25 | −17.40 |
| ATOM | 1540 | HE2 | HIS | 200S | −19.286 | 18.261 | 12.552 | 1.00 | 0.00 | 0.393 | 0.00 | 0.00 |
| ATOM | 1541 | C | HIS | 200S | −20.998 | 12.980 | 8.309 | 1.00 | 0.00 | 0.396 | 9.82 | 4.00 |
| ATOM | 1542 | O | HIS | 200S | −22.166 | 12.703 | 8.063 | 1.00 | 0.00 | −0.396 | 8.17 | −17.40 |
| ATOM | 1543 | N | ASN | 201 | −20.008 | 12.702 | 7.474 | 1.00 | 0.00 | −0.650 | 9.00 | −17.40 |
| ATOM | 1544 | HN | ASN | 201 | −19.041 | 12.954 | 7.724 | 1.00 | 0.00 | 0.440 | 0.00 | 0.00 |
| ATOM | 1545 | CA | ASN | 201 | −20.277 | 12.045 | 6.212 | 1.00 | 0.00 | 0.158 | 9.40 | 4.00 |
| ATOM | 1546 | HA | ASN | 201 | −21.274 | 12.290 | 5.848 | 1.00 | 0.00 | 0.053 | 0.00 | 0.00 |
| ATOM | 1547 | CB | ASN | 201 | −19.311 | 12.564 | 5.156 | 1.00 | 0.00 | −0.106 | 12.77 | 4.00 |
| ATOM | 1548 | HB1 | ASN | 201 | −19.530 | 12.150 | 4.171 | 1.00 | 0.00 | 0.053 | 0.00 | 0.00 |
| ATOM | 1549 | HB2 | ASN | 201 | −18.279 | 12.303 | 5.392 | 1.00 | 0.00 | 0.053 | 0.00 | 0.00 |
| ATOM | 1550 | CG | ASN | 201 | −19.375 | 14.069 | 5.033 | 1.00 | 0.00 | 0.396 | 9.82 | 4.00 |
| ATOM | 1551 | OD1 | ASN | 201 | −20.168 | 14.612 | 4.256 | 1.00 | 0.00 | −0.396 | 8.17 | −17.40 |
| ATOM | 1552 | ND2 | ASN | 201 | −18.563 | 14.760 | 5.827 | 1.00 | 0.00 | −0.879 | 13.25 | −17.40 |
| ATOM | 1553 | HD2 | ASN | 201 | −17.917 | 14.265 | 6.459 | 1.00 | 0.00 | 0.440 | 0.00 | 0.00 |
| ATOM | 1554 | HD2 | ASN | 201 | −18.579 | 15.789 | 5.809 | 1.00 | 0.00 | 0.440 | 0.00 | 0.00 |
| ATOM | 1555 | C | ASN | 201 | −20.192 | 10.543 | 6.354 | 1.00 | 0.00 | 0.396 | 9.82 | 4.00 |
| ATOM | 1556 | O | ASN | 201 | −21.192 | 9.852 | 6.124 | 1.00 | 0.00 | −0.396 | 8.17 | −17.40 |
| ATOM | 1557 | N | PHE | 202 | −19.026 | 10.022 | 6.739 | 1.00 | 0.00 | −0.650 | 9.00 | −17.40 |
| ATOM | 1558 | HN | PHE | 202 | −18.216 | 10.634 | 6.914 | 1.00 | 0.00 | 0.440 | 0.00 | 0.00 |
| ATOM | 1559 | CA | PHE | 202 | −18.906 | 8.581 | 6.910 | 1.00 | 0.00 | 0.158 | 9.40 | 4.00 |
| ATOM | 1560 | HA | PHE | 202 | −19.282 | 8.065 | 6.026 | 1.00 | 0.00 | 0.053 | 0.00 | 0.00 |
| ATOM | 1561 | CB | PHE | 202 | −17.439 | 8.151 | 7.047 | 1.00 | 0.00 | −0.106 | 12.77 | 4.00 |
| ATOM | 1562 | HB1 | PHE | 202 | −17.315 | 7.099 | 7.305 | 1.00 | 0.00 | 0.053 | 0.00 | 0.00 |
| ATOM | 1563 | HB2 | PHE | 202 | −16.894 | 8.697 | 7.816 | 1.00 | 0.00 | 0.053 | 0.00 | 0.00 |
| ATOM | 1564 | CG | PHE | 202 | −16.632 | 8.343 | 5.783 | 1.00 | 0.00 | 0.000 | 7.26 | 0.60 |
| ATOM | 1565 | CD1 | PHE | 202 | −15.910 | 9.511 | 5.569 | 1.00 | 0.00 | −0.127 | 10.80 | 0.60 |
| ATOM | 1566 | HD1 | PHE | 202 | −15.916 | 10.292 | 6.329 | 1.00 | 0.00 | 0.127 | 0.00 | 0.00 |
| ATOM | 1567 | CD2 | PHE | 202 | −16.609 | 7.355 | 4.801 | 1.00 | 0.00 | −0.127 | 10.80 | 0.60 |
| ATOM | 1568 | HD2 | PHE | 202 | −17.169 | 6.432 | 4.952 | 1.00 | 0.00 | 0.127 | 0.00 | 0.00 |
| ATOM | 1569 | CE1 | PHE | 202 | −15.173 | 9.701 | 4.390 | 1.00 | 0.00 | −0.127 | 10.80 | 0.60 |
| ATOM | 1570 | HE1 | PHE | 202 | −14.615 | 10.624 | 4.237 | 1.00 | 0.00 | 0.127 | 0.00 | 0.00 |
| ATOM | 1571 | CE2 | PHE | 202 | −15.873 | 7.534 | 3.620 | 1.00 | 0.00 | −0.127 | 10.80 | 0.60 |
| ATOM | 1572 | HE2 | PHE | 202 | −15.862 | 6.752 | 2.860 | 1.00 | 0.00 | 0.127 | 0.00 | 0.00 |
| ATOM | 1573 | CZ | PHE | 202 | −15.156 | 8.710 | 3.419 | 1.00 | 0.00 | −0.127 | 10.80 | 0.60 |
| ATOM | 1574 | HZ | PHE | 202 | −14.583 | 8.851 | 2.502 | 1.00 | 0.00 | 0.127 | 0.00 | 0.00 |
| ATOM | 1575 | C | PHE | 202 | −19.710 | 8.127 | 8.123 | 1.00 | 0.00 | 0.396 | 9.82 | 4.00 |
| ATOM | 1576 | O | PHE | 202 | −20.163 | 6.977 | 8.178 | 1.00 | 0.00 | −0.396 | 8.17 | −17.40 |
| ATOM | 1577 | N | ASN | 203 | −19.874 | 9.028 | 9.090 | 1.00 | 0.00 | −0.650 | 9.00 | −17.40 |
| ATOM | 1578 | HN | ASN | 203 | −19.429 | 9.951 | 8.984 | 1.00 | 0.00 | 0.440 | 0.00 | 0.00 |
| ATOM | 1579 | CA | ASN | 203 | −20.661 | 8.761 | 10.299 | 1.00 | 0.00 | 0.158 | 9.40 | 4.00 |
| ATOM | 1580 | HA | ASN | 203 | −20.675 | 9.668 | 10.902 | 1.00 | 0.00 | 0.053 | 0.00 | 0.00 |
| ATOM | 1581 | CB | ASN | 203 | −22.116 | 8.473 | 9.888 | 1.00 | 0.00 | −0.106 | 12.77 | 4.00 |
| ATOM | 1582 | HB1 | ASN | 203 | −22.247 | 7.482 | 9.451 | 1.00 | 0.00 | 0.053 | 0.00 | 0.00 |
| ATOM | 1583 | HB2 | ASN | 203 | −22.496 | 9.175 | 9.146 | 1.00 | 0.00 | 0.053 | 0.00 | 0.00 |
| ATOM | 1584 | CG | ASN | 203 | −23.090 | 8.541 | 11.057 | 1.00 | 0.00 | 0.396 | 9.82 | 4.00 |
| ATOM | 1585 | OD1 | ASN | 203 | −24.155 | 7.916 | 11.018 | 1.00 | 0.00 | −0.396 | 8.17 | −17.40 |
| ATOM | 1586 | ND2 | ASN | 203 | −22.743 | 9.309 | 12.096 | 1.00 | 0.00 | −0.879 | 13.25 | −17.40 |
| ATOM | 1587 | HD2 | ASN | 203 | −21.844 | 9.812 | 12.086 | 1.00 | 0.00 | 0.440 | 0.00 | 0.00 |
| ATOM | 1588 | HD2 | ASN | 203 | −23.374 | 9.396 | 12.905 | 1.00 | 0.00 | 0.440 | 0.00 | 0.00 |
| ATOM | 1589 | C | ASN | 203 | −20.117 | 7.625 | 11.184 | 1.00 | 0.00 | 0.396 | 9.82 | 4.00 |
| ATOM | 1590 | O | ASN | 203 | −20.870 | 6.768 | 11.660 | 1.00 | 0.00 | −0.396 | 8.17 | −17.40 |
| ATOM | 1591 | N | TYR | 204 | −18.807 | 7.631 | 11.406 | 1.00 | 0.00 | −0.650 | 9.00 | −17.40 |
| ATOM | 1592 | HN | TYR | 204 | −18.234 | 8.370 | 10.974 | 1.00 | 0.00 | 0.440 | 0.00 | 0.00 |
| ATOM | 1593 | CA | TYR | 204 | −18.149 | 6.630 | 12.238 | 1.00 | 0.00 | 0.158 | 9.40 | 4.00 |
| ATOM | 1594 | HA | TYR | 204 | −18.629 | 5.667 | 12.062 | 1.00 | 0.00 | 0.053 | 0.00 | 0.00 |
| ATOM | 1595 | CB | TYR | 204 | −16.669 | 6.523 | 11.862 | 1.00 | 0.00 | −0.106 | 12.77 | 4.00 |
| ATOM | 1596 | HB1 | TYR | 204 | −16.159 | 5.953 | 12.639 | 1.00 | 0.00 | 0.053 | 0.00 | 0.00 |
| ATOM | 1597 | HB2 | TYR | 204 | −16.259 | 7.531 | 11.793 | 1.00 | 0.00 | 0.053 | 0.00 | 0.00 |
| ATOM | 1598 | CG | TYR | 204 | −16.409 | 5.832 | 10.541 | 1.00 | 0.00 | 0.00 | 07.26 | 0.60 |
| ATOM | 1599 | CD1 | TYR | 204 | −15.745 | 6.487 | 9.499 | 1.00 | 0.00 | −0.127 | 10.80 | 0.60 |
| ATOM | 1600 | HD1 | TYR | 204 | −15.440 | 7.525 | 9.626 | 1.00 | 0.00 | 0.127 | 0.00 | 0.00 |
| ATOM | 1601 | CE1 | TYR | 204 | −15.465 | 5.828 | 8.294 | 1.00 | 0.00 | −0.127 | 10.80 | 0.60 |
| ATOM | 1602 | HE1 | TYR | 204 | −14.944 | 6.349 | 7.491 | 1.00 | 0.00 | 0.127 | 0.00 | 0.00 |
| ATOM | 1603 | CD2 | TYR | 204 | −16.792 | 4.505 | 10.344 | 1.00 | 0.00 | −0.127 | 10.80 | 0.60 |
| ATOM | 1604 | HD2 | TYR | 204 | −17.312 | 3.974 | 11.141 | 1.00 | 0.00 | 0.127 | 0.00 | 0.00 |
| ATOM | 1605 | CE2 | TYR | 204 | −16.519 | 3.843 | 9.140 | 1.00 | 0.00 | −0.127 | 10.80 | 0.60 |
| ATOM | 1606 | HE2 | TYR | 204 | −16.830 | 2.807 | 9.004 | 1.00 | 0.00 | 0.127 | 0.00 | 0.00 |
| ATOM | 1607 | CZ | TYR | 204 | −15.857 | 4.501 | 8.130 | 1.00 | 0.00 | 0.026 | 7.26 | 0.60 |
| ATOM | 1608 | OH | TYR | 204 | −15.548 | 3.809 | 6.983 | 1.00 | 0.00 | −0.451 | 10.94 | −17.40 |
| ATOM | 1609 | HH | TYR | 204 | −15.959 | 4.289 | 6.170 | 1.00 | 0.00 | 0.424 | 0.00 | 0.00 |
| ATOM | 1610 | C | TYR | 204 | −18.298 | 7.044 | 13.696 | 1.00 | 0.00 | 0.396 | 9.82 | 4.00 |
| ATOM | 1611 | O | TYR | 204 | −18.378 | 8.233 | 14.000 | 1.00 | 0.00 | −0.396 | 8.17 | −17.40 |
| ATOM | 1612 | N | PRO | 205 | −18.300 | 6.068 | 14.620 | 1.00 | 0.00 | −0.422 | 9.00 | −17.40 |
| ATOM | 1613 | CD | PRO | 205 | −18.114 | 4.621 | 14.383 | 1.00 | 0.00 | 0.105 | 12.77 | 4.00 |
| ATOM | 1614 | HD1 | PRO | 205 | −17.200 | 4.427 | 13.821 | 1.00 | 0.00 | 0.053 | 0.00 | 0.00 |
| ATOM | 1615 | HD2 | PRO | 205 | −18.945 | 4.204 | 13.815 | 1.00 | 0.00 | 0.053 | 0.00 | 0.00 |
| ATOM | 1616 | CA | PRO | 205 | −18.451 | 6.338 | 16.049 | 1.00 | 0.00 | 0.158 | 9.40 | 4.00 |
| ATOM | 1617 | HA | PRO | 205 | −19.201 | 7.118 | 16.171 | 1.00 | 0.00 | 0.053 | 0.00 | 0.00 |
| ATOM | 1618 | CB | PRO | 205 | −18.920 | 5.000 | 16.583 | 1.00 | 0.00 | −0.106 | 12.77 | 4.00 |
| ATOM | 1619 | HB1 | PRO | 205 | −19.981 | 4.841 | 16.390 | 1.00 | 0.00 | 0.053 | 0.00 | 0.00 |
| ATOM | 1620 | HB2 | PRO | 205 | −18.770 | 4.924 | 17.660 | 1.00 | 0.00 | 0.053 | 0.00 | 0.00 |
| ATOM | 1621 | CG | PRO | 205 | −18.046 | 4.053 | 15.811 | 1.00 | 0.00 | −0.106 | 12.77 | 4.00 |
| ATOM | 1622 | HG1 | PRO | 205 | −18.426 | 3.032 | 15.864 | 1.00 | 0.00 | 0.053 | 0.00 | 0.00 |
| ATOM | 1623 | HG2 | PRO | 205 | −17.028 | 4.047 | 16.201 | 1.00 | 0.00 | 0.053 | 0.00 | 0.00 |
| ATOM | 1624 | C | PRO | 205 | −17.179 | 6.810 | 16.739 | 1.00 | 0.00 | 0.396 | 9.82 | 4.00 |
| ATOM | 1625 | O | PRO | 205 | −17.216 | 7.255 | 17.886 | 1.00 | 0.00 | −0.396 | 8.17 | −17.40 |
| ATOM | 1626 | N | ALA | 206 | −16.059 | 6.705 | 16.029 | 1.00 | 0.00 | −0.650 | 9.00 | −17.40 |
| ATOM | 1627 | HN | ALA | 206 | −16.110 | 6.340 | 15.067 | 1.00 | 0.00 | 0.440 | 0.00 | 0.00 |
| ATOM | 1628 | CA | ALA | 206 | −14.762 | 7.090 | 16.569 | 1.00 | 0.00 | 0.158 | 9.40 | 4.00 |
| ATOM | 1629 | HA | ALA | 206 | −14.844 | 8.098 | 16.974 | 1.00 | 0.00 | 0.053 | 0.00 | 0.00 |
| ATOM | 1630 | CB | ALA | 206 | −14.365 | 6.119 | 17.681 | 1.00 | 0.00 | −0.159 | 16.15 | 4.00 |
| ATOM | 1631 | HB1 | ALA | 206 | −13.394 | 6.406 | 18.085 | 1.00 | 0.00 | 0.053 | 0.00 | 0.00 |
| ATOM | 1632 | HB2 | ALA | 206 | −15.112 | 6.149 | 18.474 | 1.00 | 0.00 | 0.053 | 0.00 | 0.00 |
| ATOM | 1633 | HB3 | ALA | 206 | −14.304 | 5.108 | 17.277 | 1.00 | 0.00 | 0.053 | 0.00 | 0.00 |
| ATOM | 1634 | C | ALA | 206 | −13.739 | 7.050 | 15.438 | 1.00 | 0.00 | 0.396 | 9.82 | 4.00 |
| ATOM | 1635 | O | ALA | 206 | −14.068 | 6.651 | 14.311 | 1.00 | 0.00 | −0.396 | 8.17 | −17.40 |
| ATOM | 1636 | N | ALA | 207 | −12.509 | 7.480 | 15.722 | 1.00 | 0.00 | −0.650 | 9.00 | −17.40 |
| ATOM | 1637 | HN | ALA | 207 | −12.304 | 7.833 | 16.667 | 1.00 | 0.00 | 0.440 | 0.00 | 0.00 |
| ATOM | 1638 | CA | ALA | 207 | −11.442 | 7.461 | 14.718 | 1.00 | 0.00 | 0.158 | 9.40 | 4.00 |
| ATOM | 1639 | HA | ALA | 207 | −11.420 | 6.542 | 14.132 | 1.00 | 0.00 | 0.053 | 0.00 | 0.00 |
| ATOM | 1640 | CB | ALA | 207 | −11.669 | 8.581 | 13.708 | 1.00 | 0.00 | −0.159 | 16.15 | 4.00 |
| ATOM | 1641 | HB1 | ALA | 207 | −10.874 | 8.565 | 12.962 | 1.00 | 0.00 | 0.053 | 0.00 | 0.00 |
| ATOM | 1642 | HB2 | ALA | 207 | −12.631 | 8.437 | 13.216 | 1.00 | 0.00 | 0.053 | 0.00 | 0.00 |
| ATOM | 1643 | HB3 | ALA | 207 | −11.664 | 9.541 | 14.223 | 1.00 | 0.00 | 0.053 | 0.00 | 0.00 |
| ATOM | 1644 | C | ALA | 207 | −10.042 | 7.593 | 15.324 | 1.00 | 0.00 | 0.396 | 9.82 | 4.00 |
| ATOM | 1645 | O | ALA | 207 | −9.849 | 8.232 | 16.369 | 1.00 | 0.00 | −0.396 | 8.17 | −17.40 |
| ATOM | 1646 | N | VAL | 208 | −9.062 | 6.973 | 14.676 | 1.00 | 0.00 | −0.650 | 9.00 | −17.40 |
| ATOM | 1647 | HN | VAL | 208 | −9.283 | 6.402 | 13.847 | 1.00 | 0.00 | 0.440 | 0.00 | 0.00 |
| ATOM | 1648 | CA | VAL | 208 | −7.680 | 7.087 | 15.118 | 1.00 | 0.00 | 0.158 | 9.40 | 4.00 |
| ATOM | 1649 | HA | VAL | 208 | −7.748 | 7.519 | 16.116 | 1.00 | 0.00 | 0.053 | 0.00 | 0.00 |
| ATOM | 1650 | CB | VAL | 208 | −6.951 | 5.709 | 15.173 | 1.00 | 0.00 | −0.053 | 9.40 | 4.00 |
| ATOM | 1651 | HB | VAL | 208 | −7.041 | 5.192 | 14.217 | 1.00 | 0.00 | 0.053 | 0.00 | 0.00 |
| ATOM | 1652 | CG1 | VAL | 208 | −5.449 | 5.905 | 15.484 | 1.00 | 0.00 | −0.159 | 16.15 | 4.00 |
| ATOM | 1653 | HG1 | VAL | 208 | −4.954 | 4.934 | 15.519 | 1.00 | 0.00 | 0.053 | 0.00 | 0.00 |
| ATOM | 1654 | HG1 | VAL | 208 | −4.993 | 6.516 | 14.705 | 1.00 | 0.00 | 0.053 | 0.00 | 0.00 |
| ATOM | 1655 | HG1 | VAL | 208 | −5.338 | 6.402 | 16.447 | 1.00 | 0.00 | 0.053 | 0.00 | 0.00 |
| ATOM | 1656 | CG2 | VAL | 208 | −7.569 | 4.837 | 16.278 | 1.00 | 0.00 | −0.159 | 16.15 | 4.00 |
| ATOM | 1657 | HG2 | VAL | 208 | −7.056 | 3.875 | 16.312 | 1.00 | 0.00 | 0.053 | 0.00 | 0.00 |
| ATOM | 1658 | HG2 | VAL | 208 | −7.463 | 5.339 | 17.239 | 1.00 | 0.00 | 0.053 | 0.00 | 0.00 |
| ATOM | 1659 | HG2 | VAL | 208 | −8.626 | 4.677 | 16.066 | 1.00 | 0.00 | 0.053 | 0.00 | 0.00 |
| ATOM | 1660 | C | VAL | 208 | −7.023 | 8.005 | 14.086 | 1.00 | 0.00 | 0.396 | 9.82 | 4.00 |
| ATOM | 1661 | O | VAL | 208 | −6.944 | 7.678 | 12.895 | 1.00 | 0.00 | −0.396 | 8.17 | −17.40 |
| ATOM | 1662 | N | VAL | 209 | −6.573 | 9.161 | 14.561 | 1.00 | 0.00 | −0.650 | 9.00 | −17.40 |
| ATOM | 1663 | HN | VAL | 209 | −6.686 | 9.355 | 15.566 | 1.00 | 0.00 | 0.440 | 0.00 | 0.00 |
| ATOM | 1664 | CA | VAL | 209 | −5.924 | 10.171 | 13.726 | 1.00 | 0.00 | 0.158 | 9.40 | 4.00 |
| ATOM | 1665 | HA | VAL | 209 | −6.351 | 10.147 | 12.723 | 1.00 | 0.00 | 0.053 | 0.00 | 0.00 |
| ATOM | 1666 | CB | VAL | 209 | −6.101 | 11.597 | 14.334 | 1.00 | 0.00 | −0.053 | 9.40 | 4.00 |
| ATOM | 1667 | HB | VAL | 209 | −5.550 | 11.668 | 15.271 | 1.00 | 0.00 | 0.053 | 0.00 | 0.00 |
| ATOM | 1668 | CG1 | VAL | 209 | −5.572 | 12.638 | 13.364 | 1.00 | 0.00 | −0.159 | 16.15 | 4.00 |
| ATOM | 1669 | HG1 | VAL | 209 | −5.697 | 13.632 | 13.793 | 1.00 | 0.00 | 0.053 | 0.00 | 0.00 |
| ATOM | 1670 | HG1 | VAL | 209 | −4.514 | 12.455 | 13.175 | 1.00 | 0.00 | 0.053 | 0.00 | 0.00 |
| ATOM | 1671 | HG1 | VAL | 209 | −6.123 | 12.575 | 12.426 | 1.00 | 0.00 | 0.053 | 0.00 | 0.00 |
| ATOM | 1672 | CG2 | VAL | 209 | −7.567 | 11.869 | 14.664 | 1.00 | 0.00 | −0.159 | 16.15 | 4.00 |
| ATOM | 1673 | HG2 | VAL | 209 | −7.665 | 12.868 | 15.086 | 1.00 | 0.00 | 0.053 | 0.00 | 0.00 |
| ATOM | 1674 | HG2 | VAL | 209 | −8.163 | 11.799 | 13.754 | 1.00 | 0.00 | 0.053 | 0.00 | 0.00 |
| ATOM | 1675 | HG2 | VAL | 209 | −7.919 | 11.133 | 15.387 | 1.00 | 0.00 | 0.053 | 0.00 | 0.00 |
| ATOM | 1676 | C | VAL | 209 | −4.418 | 9.882 | 13.643 | 1.00 | 0.00 | 0.396 | 9.82 | 4.00 |
| ATOM | 1677 | O | VAL | 209 | −3.726 | 9.828 | 14.668 | 1.00 | 0.00 | −0.396 | 8.17 | −17.40 |
| ATOM | 1678 | N | VAL | 210 | −3.918 | 9.748 | 12.419 | 1.00 | 0.00 | −0.650 | 9.00 | −17.40 |
| ATOM | 1679 | HN | VAL | 210 | −4.547 | 9.856 | 11.611 | 1.00 | 0.00 | 0.440 | 0.00 | 0.00 |
| ATOM | 1680 | CA | VAL | 210 | −2.504 | 9.452 | 12.177 | 1.00 | 0.00 | 0.158 | 9.40 | 4.00 |
| ATOM | 1681 | HA | VAL | 210 | −1.907 | 9.566 | 13.082 | 1.00 | 0.00 | 0.053 | 0.00 | 0.00 |
| ATOM | 1682 | CB | VAL | 210 | −2.329 | 8.005 | 11.668 | 1.00 | 0.00 | −0.053 | 9.40 | 4.00 |
| ATOM | 1683 | HB | VAL | 210 | −2.855 | 7.884 | 10.721 | 1.00 | 0.00 | 0.053 | 0.00 | 0.00 |
| ATOM | 1684 | CG1 | VAL | 210 | −0.851 | 7.712 | 11.464 | 1.00 | 0.00 | −0.159 | 16.15 | 4.00 |
| ATOM | 1685 | HG1 | VAL | 210 | −0.728 | 6.690 | 11.104 | 1.00 | 0.00 | 0.053 | 0.00 | 0.00 |
| ATOM | 1686 | HG1 | VAL | 210 | −0.440 | 8.406 | 10.730 | 1.00 | 0.00 | 0.053 | 0.00 | 0.00 |
| ATOM | 1687 | HG1 | VAL | 210 | −0.322 | 7.829 | 12.410 | 1.00 | 0.00 | 0.053 | 0.00 | 0.00 |
| ATOM | 1688 | CG2 | VAL | 210 | −2.961 | 7.015 | 12.663 | 1.00 | 0.00 | −0.159 | 16.15 | 4.00 |
| ATOM | 1689 | HG2 | VAL | 210 | −2.832 | 5.997 | 12.295 | 1.00 | 0.00 | 0.053 | 0.00 | 0.00 |
| ATOM | 1690 | HG2 | VAL | 210 | −2.474 | 7.114 | 13.633 | 1.00 | 0.00 | 0.053 | 0.00 | 0.00 |
| ATOM | 1691 | HG2 | VAL | 210 | −4.024 | 7.231 | 12.766 | 1.00 | 0.00 | 0.053 | 0.00 | 0.00 |
| ATOM | 1692 | C | VAL | 210 | −1.905 | 10.379 | 11.124 | 1.00 | 0.00 | 0.396 | 9.82 | 4.00 |
| ATOM | 1693 | O | VAL | 210 | −2.327 | 10.357 | 9.971 | 1.00 | 0.00 | −0.396 | 8.17 | −17.40 |
| ATOM | 1694 | N | GLU | 211 | −0.925 | 11.191 | 11.505 | 1.00 | 0.00 | −0.650 | 9.00 | −17.40 |
| ATOM | 1695 | HN | GLU | 211 | −0.595 | 11.193 | 12.481 | 1.00 | 0.00 | 0.440 | 0.00 | 0.00 |
| ATOM | 1696 | CA | GLU | 211 | −0.326 | 12.076 | 10.520 | 1.00 | 0.00 | 0.158 | 9.40 | 4.00 |
| ATOM | 1697 | HA | GLU | 211 | −1.107 | 12.559 | 9.933 | 1.00 | 0.00 | 0.053 | 0.00 | 0.00 |
| ATOM | 1698 | CB | GLU | 211 | 0.404 | 13.225 | 11.220 | 1.00 | 0.00 | −0.106 | 12.77 | 4.00 |
| ATOM | 1699 | HB1 | GLU | 211 | 0.929 | 13.831 | 10.482 | 1.00 | 0.00 | 0.053 | 0.00 | 0.00 |
| ATOM | 1700 | HB2 | GLU | 211 | 1.124 | 12.824 | 11.932 | 1.00 | 0.00 | 0.053 | 0.00 | 0.00 |
| ATOM | 1701 | CG | GLU | 211 | −0.605 | 14.102 | 11.965 | 1.00 | 0.00 | −0.106 | 12.77 | 4.00 |
| ATOM | 1702 | HG1 | GLU | 211 | −1.101 | 13.490 | 12.718 | 1.00 | 0.00 | 0.053 | 0.00 | 0.00 |
| ATOM | 1703 | HG2 | GLU | 211 | −1.328 | 14.482 | 11.243 | 1.00 | 0.00 | 0.053 | 0.00 | 0.00 |
| ATOM | 1704 | CD | GLU | 211 | 0.012 | 15.286 | 12.667 | 1.00 | 0.00 | 0.399 | 9.82 | 4.00 |
| ATOM | 1705 | OE1 | GLU | 211 | 1.175 | 15.180 | 13.104 | 1.00 | 0.00 | −0.396 | 8.17 | −18.95 |
| ATOM | 1706 | OE2 | GLU | 211 | −0.681 | 16.324 | 12.799 | 1.00 | 0.00 | −0.427 | 8.17 | −18.95 |
| ATOM | 1707 | HE2 | GLU | 211 | −0.139 | 17.036 | 13.309 | 1.00 | 0.00 | 0.424 | 0.00 | 0.00 |
| ATOM | 1708 | C | GLU | 211 | 0.583 | 11.270 | 9.579 | 1.00 | 0.00 | 0.396 | 9.82 | 4.00 |
| ATOM | 1709 | O | GLU | 211 | 1.101 | 10.209 | 9.955 | 1.00 | 0.00 | −0.396 | 8.17 | −17.40 |
| ATOM | 1710 | N | ASP | 212P | 0.749 | 11.778 | 8.360 | 1.00 | 0.00 | −0.650 | 9.00 | −17.40 |
| ATOM | 1711 | HN | ASP | 212P | 0.304 | 12.681 | 8.143 | 1.00 | 0.00 | 0.440 | 0.00 | 0.00 |
| ATOM | 1712 | CA | ASP | 212P | 1.528 | 11.122 | 7.312 | 1.00 | 0.00 | 0.158 | 9.40 | 4.00 |
| ATOM | 1713 | HA | ASP | 212P | 1.043 | 10.171 | 7.090 | 1.00 | 0.00 | 0.053 | 0.00 | 0.00 |
| ATOM | 1714 | CB | ASP | 212P | 1.457 | 11.959 | 6.028 | 1.00 | 0.00 | −0.336 | 12.77 | 4.00 |
| ATOM | 1715 | HB1 | ASP | 212P | 0.430 | 12.140 | 5.709 | 1.00 | 0.00 | 0.053 | 0.00 | 0.00 |
| ATOM | 1716 | HB2 | ASP | 212P | 1.960 | 11.475 | 5.191 | 1.00 | 0.00 | 0.053 | 0.00 | 0.00 |
| ATOM | 1717 | CG | ASP | 212P | 2.108 | 13.326 | 6.193 | 1.00 | 0.00 | 0.297 | 9.82 | 4.00 |
| ATOM | 1718 | OD1 | ASP | 212P | 1.842 | 13.976 | 7.210 | 1.00 | 0.00 | −0.534 | 8.17 | −18.95 |
| ATOM | 1719 | OD2 | ASP | 212P | 2.876 | 13.758 | 5.318 | 1.00 | 0.00 | −0.534 | 8.17 | −18.95 |
| ATOM | 1720 | C | ASP | 212P | 2.989 | 10.804 | 7.615 | 1.00 | 0.00 | 0.396 | 9.82 | 4.00 |
| ATOM | 1721 | O | ASP | 212P | 3.608 | 10.009 | 6.888 | 1.00 | 0.00 | −0.396 | 8.17 | −17.40 |
| ATOM | 1722 | N | ASP | 213P | 3.552 | 11.406 | 8.663 | 1.00 | 0.00 | −0.650 | 9.00 | −17.40 |
| ATOM | 1723 | HN | ASP | 213P | 3.000 | 12.056 | 9.240 | 1.00 | 0.00 | 0.440 | 0.00 | 0.00 |
| ATOM | 1724 | CA | ASP | 213P | 4.947 | 11.144 | 8.993 | 1.00 | 0.00 | 0.158 | 9.40 | 4.00 |
| ATOM | 1725 | HA | ASP | 213P | 5.412 | 10.641 | 8.145 | 1.00 | 0.00 | 0.053 | 0.00 | 0.00 |
| ATOM | 1726 | CB | ASP | 213P | 5.727 | 12.470 | 9.124 | 1.00 | 0.00 | −0.336 | 12.77 | 4.00 |
| ATOM | 1727 | HB1 | ASP | 213P | 5.731 | 13.064 | 8.210 | 1.00 | 0.00 | 0.053 | 0.00 | 0.00 |
| ATOM | 1728 | HB2 | ASP | 213P | 6.777 | 12.333 | 9.379 | 1.00 | 0.00 | 0.053 | 0.00 | 0.00 |
| ATOM | 1729 | CG | ASP | 213P | 5.173 | 13.400 | 10.202 | 1.00 | 0.00 | 0.297 | 9.82 | 4.00 |
| ATOM | 1730 | OD1 | ASP | 213P | 4.080 | 13.123 | 10.751 | 1.00 | 0.00 | −0.534 | 8.17 | −18.95 |
| ATOM | 1731 | OD2 | ASP | 213P | 5.837 | 14.431 | 10.497 | 1.00 | 0.00 | −0.534 | 8.17 | −18.95 |
| ATOM | 1732 | C | ASP | 213P | 5.139 | 10.253 | 10.229 | 1.00 | 0.00 | 0.396 | 9.82 | 4.00 |
| ATOM | 1733 | O | ASP | 213P | 6.180 | 10.289 | 10.892 | 1.00 | 0.00 | −0.396 | 8.17 | −17.40 |
| ATOM | 1734 | N | LEU | 214 | 4.135 | 9.433 | 10.524 | 1.00 | 0.00 | −0.650 | 9.00 | −17.40 |
| ATOM | 1735 | HN | LEU | 214 | 3.283 | 9.441 | 9.944 | 1.00 | 0.00 | 0.440 | 0.00 | 0.00 |
| ATOM | 1736 | CA | LEU | 214 | 4.225 | 8.520 | 11.662 | 1.00 | 0.00 | 0.158 | 9.40 | 4.00 |
| ATOM | 1737 | HA | LEU | 214 | 5.134 | 8.706 | 12.233 | 1.00 | 0.00 | 0.053 | 0.00 | 0.00 |
| ATOM | 1738 | CB | LEU | 214 | 3.028 | 8.678 | 12.613 | 1.00 | 0.00 | −0.106 | 12.77 | 4.00 |
| ATOM | 1739 | HB1 | LEU | 214 | 3.237 | 8.080 | 13.500 | 1.00 | 0.00 | 0.053 | 0.00 | 0.00 |
| ATOM | 1740 | HB2 | LEU | 214 | 2.145 | 8.315 | 12.086 | 1.00 | 0.00 | 0.053 | 0.00 | 0.00 |
| ATOM | 1741 | CG | LEU | 214 | 2.682 | 10.085 | 13.114 | 1.00 | 0.00 | −0.053 | 9.40 | 4.00 |
| ATOM | 1742 | HG | LEU | 214 | 2.413 | 10.705 | 12.258 | 1.00 | 0.00 | 0.053 | 0.00 | 0.00 |
| ATOM | 1743 | CD1 | LEU | 214 | 1.500 | 9.985 | 14.087 | 1.00 | 0.00 | −0.159 | 16.15 | 4.00 |
| ATOM | 1744 | HD1 | LEU | 214 | 1.243 | 10.979 | 14.451 | 1.00 | 0.00 | 0.053 | 0.00 | 0.00 |
| ATOM | 1745 | HD1 | LEU | 214 | 0.640 | 9.554 | 13.572 | 1.00 | 0.00 | 0.053 | 0.00 | 0.00 |
| ATOM | 1746 | HD1 | LEU | 214 | 1.774 | 9.349 | 14.929 | 1.00 | 0.00 | 0.053 | 0.00 | 0.00 |
| ATOM | 1747 | CD2 | LEU | 214 | 3.899 | 10.708 | 13.793 | 1.00 | 0.00 | −0.159 | 16.15 | 4.00 |
| ATOM | 1748 | HD2 | LEU | 214 | 3.646 | 11.707 | 14.146 | 1.00 | 0.00 | 0.053 | 0.00 | 0.00 |
| ATOM | 1749 | HD2 | LEU | 214 | 4.200 | 10.089 | 14.638 | 1.00 | 0.00 | 0.053 | 0.00 | 0.00 |
| ATOM | 1750 | HD2 | LEU | 214 | 4.720 | 10.772 | 13.079 | 1.00 | 0.00 | 0.053 | 0.00 | 0.00 |
| ATOM | 1751 | C | LEU | 214 | 4.246 | 7.077 | 11.190 | 1.00 | 0.00 | 0.396 | 9.82 | 4.00 |
| ATOM | 1752 | O | LEU | 214 | 3.483 | 6.681 | 10.289 | 1.00 | 0.00 | −0.396 | 8.17 | −17.40 |
| ATOM | 1753 | N | GLU | 215 | 5.125 | 6.286 | 11.782 | 1.00 | 0.00 | −0.650 | 9.00 | −17.40 |
| ATOM | 1754 | HN | GLU | 215 | 5.787 | 6.679 | 12.466 | 1.00 | 0.00 | 0.440 | 0.00 | 0.00 |
| ATOM | 1755 | CA | GLU | 215 | 5.164 | 4.866 | 11.475 | 1.00 | 0.00 | 0.158 | 9.40 | 4.00 |
| ATOM | 1756 | HA | GLU | 215 | 4.584 | 4.763 | 10.557 | 1.00 | 0.00 | 0.053 | 0.00 | 0.00 |
| ATOM | 1757 | CB | GLU | 215 | 6.592 | 4.376 | 11.328 | 1.00 | 0.00 | −0.106 | 12.77 | 4.00 |
| ATOM | 1758 | HB1 | GLU | 215 | 7.178 | 4.543 | 12.231 | 1.00 | 0.00 | 0.053 | 0.00 | 0.00 |
| ATOM | 1759 | HB2 | GLU | 215 | 7.117 | 4.879 | 10.516 | 1.00 | 0.00 | 0.053 | 0.00 | 0.00 |
| ATOM | 1760 | CG | GLU | 215 | 6.658 | 2.901 | 11.037 | 1.00 | 0.00 | −0.106 | 12.77 | 4.00 |
| ATOM | 1761 | HG1 | GLU | 215 | 6.021 | 2.608 | 10.202 | 1.00 | 0.00 | 0.053 | 0.00 | 0.00 |
| ATOM | 1762 | HG2 | GLU | 215 | 6.344 | 2.290 | 11.884 | 1.00 | 0.00 | 0.053 | 0.00 | 0.00 |
| ATOM | 1763 | CD | GLU | 215 | 8.060 | 2.436 | 10.680 | 1.00 | 0.00 | 0.399 | 9.82 | 4.00 |
| ATOM | 1764 | OE1 | GLU | 215 | 8.764 | 3.177 | 9.955 | 1.00 | 0.00 | −0.396 | 8.17 | −18.95 |
| ATOM | 1765 | OE2 | GLU | 215 | 8.441 | 1.325 | 11.116 | 1.00 | 0.00 | −0.427 | 8.17 | −18.95 |
| ATOM | 1766 | HE2 | GLU | 215 | 9.398 | 1.135 | 10.785 | 1.00 | 0.00 | 0.424 | 0.00 | 0.00 |
| ATOM | 1767 | C | GLU | 215 | 4.523 | 4.186 | 12.686 | 1.00 | 0.00 | 0.396 | 9.82 | 4.00 |
| ATOM | 1768 | O | GLU | 215 | 4.885 | 4.496 | 13.824 | 1.00 | 0.00 | −0.396 | 8.17 | −17.40 |
| ATOM | 1769 | N | VAL | 216 | 3.578 | 3.278 | 12.456 | 1.00 | 0.00 | −0.650 | 9.00 | −17.40 |
| ATOM | 1770 | HN | VAL | 216 | 3.308 | 3.062 | 11.485 | 1.00 | 0.00 | 0.440 | 0.00 | 0.00 |
| ATOM | 1771 | CA | VAL | 216 | 2.914 | 2.578 | 13.561 | 1.00 | 0.00 | 0.158 | 9.40 | 4.00 |
| ATOM | 1772 | HA | VAL | 216 | 2.839 | 3.179 | 14.466 | 1.00 | 0.00 | 0.053 | 0.00 | 0.00 |
| ATOM | 1773 | CB | VAL | 216 | 1.447 | 2.213 | 13.192 | 1.00 | 0.00 | −0.053 | 9.40 | 4.00 |
| ATOM | 1774 | HB | VAL | 216 | 0.953 | 1.719 | 14.029 | 1.00 | 0.00 | 0.053 | 0.00 | 0.00 |
| ATOM | 1775 | CG1 | VAL | 216 | 0.648 | 3.481 | 12.836 | 1.00 | 0.00 | −0.159 | 16.15 | 4.00 |
| ATOM | 1776 | HG1 | VAL | 216 | −0.375 | 3.206 | 12.580 | 1.00 | 0.00 | 0.053 | 0.00 | 0.00 |
| ATOM | 1777 | HG1 | VAL | 216 | 0.639 | 4.157 | 13.690 | 1.00 | 0.00 | 0.053 | 0.00 | 0.00 |
| ATOM | 1778 | HG1 | VAL | 216 | 1.113 | 3.977 | 11.984 | 1.00 | 0.00 | 0.053 | 0.00 | 0.00 |
| ATOM | 1779 | CG2 | VAL | 216 | 1.436 | 1.234 | 12.015 | 1.00 | 0.00 | −0.159 | 16.15 | 4.00 |
| ATOM | 1780 | HG2 | VAL | 216 | 0.406 | 0.981 | 11.761 | 1.00 | 0.00 | 0.053 | 0.00 | 0.00 |
| ATOM | 1781 | HG2 | VAL | 216 | 1.919 | 1.694 | 11.153 | 1.00 | 0.00 | 0.053 | 0.00 | 0.00 |
| ATOM | 1782 | HG2 | VAL | 216 | 1.974 | 0.327 | 12.291 | 1.00 | 0.00 | 0.053 | 0.00 | 0.00 |
| ATOM | 1783 | C | VAL | 216 | 3.637 | 1.289 | 13.981 | 1.00 | 0.00 | 0.396 | 9.82 | 4.00 |
| ATOM | 1784 | O | VAL | 216 | 4.296 | 0.638 | 13.168 | 1.00 | 0.00 | −0.396 | 8.17 | −17.40 |
| ATOM | 1785 | N | ALA | 217 | 3.524 | 0.933 | 15.264 | 1.00 | 0.00 | −0.650 | 9.00 | −17.40 |
| ATOM | 1786 | HN | ALA | 217 | 2.993 | 1.543 | 15.901 | 1.00 | 0.00 | 0.440 | 0.00 | 0.00 |
| ATOM | 1787 | CA | ALA | 217 | 4.125 | −0.291 | 15.801 | 1.00 | 0.00 | 0.158 | 9.40 | 4.00 |
| ATOM | 1788 | HA | ALA | 217 | 5.129 | −0.406 | 15.393 | 1.00 | 0.00 | 0.053 | 0.00 | 0.00 |
| ATOM | 1789 | CB | ALA | 217 | 4.205 | −0.193 | 17.325 | 1.00 | 0.00 | −0.159 | 16.15 | 4.00 |
| ATOM | 1790 | HB1 | ALA | 217 | 4.651 | −1.103 | 17.725 | 1.00 | 0.00 | 0.053 | 0.00 | 0.00 |
| ATOM | 1791 | HB2 | ALA | 217 | 4.817 | 0.664 | 17.602 | 1.00 | 0.00 | 0.053 | 0.00 | 0.00 |
| ATOM | 1792 | HB3 | ALA | 217 | 3.202 | −0.070 | 17.735 | 1.00 | 0.00 | 0.053 | 0.00 | 0.00 |
| ATOM | 1793 | C | ALA | 217 | 3.261 | −1.496 | 15.401 | 1.00 | 0.00 | 0.396 | 9.82 | 4.00 |
| ATOM | 1794 | O | ALA | 217 | 2.107 | −1.331 | 15.024 | 1.00 | 0.00 | −0.396 | 8.17 | −17.40 |
| ATOM | 1795 | N | PRO | 218 | 3.800 | −2.727 | 15.515 | 1.00 | 0.00 | −0.422 | 9.00 | −17.40 |
| ATOM | 1796 | CD | PRO | 218 | 5.177 | −3.030 | 15.961 | 1.00 | 0.00 | 0.105 | 12.77 | 4.00 |
| ATOM | 1797 | HD1 | PRO | 218 | 5.249 | −3.019 | 17.048 | 1.00 | 0.00 | 0.053 | 0.00 | 0.00 |
| ATOM | 1798 | HD2 | PRO | 218 | 5.885 | −2.297 | 15.573 | 1.00 | 0.00 | 0.053 | 0.00 | 0.00 |
| ATOM | 1799 | CA | PRO | 218 | 3.080 | −3.962 | 15.163 | 1.00 | 0.00 | 0.158 | 9.40 | 4.00 |
| ATOM | 1800 | HA | PRO | 218 | 2.826 | −4.031 | 14.105 | 1.00 | 0.00 | 0.053 | 0.00 | 0.00 |
| ATOM | 1801 | CB | PRO | 218 | 4.076 | −5.071 | 15.555 | 1.00 | 0.00 | −0.106 | 12.77 | 4.00 |
| ATOM | 1802 | HB1 | PRO | 218 | 3.972 | −5.938 | 14.902 | 1.00 | 0.00 | 0.053 | 0.00 | 0.00 |
| ATOM | 1803 | HB2 | PRO | 218 | 3.909 | −5.401 | 16.580 | 1.00 | 0.00 | 0.053 | 0.00 | 0.00 |
| ATOM | 1804 | CG | PRO | 218 | 5.421 | −4.406 | 15.393 | 1.00 | 0.00 | −0.106 | 12.77 | 4.00 |
| ATOM | 1805 | HG1 | PRO | 218 | 5.720 | −4.370 | 14.345 | 1.00 | 0.00 | 0.053 | 0.00 | 0.00 |
| ATOM | 1806 | HG2 | PRO | 218 | 6.195 | −4.943 | 15.939 | 1.00 | 0.00 | 0.053 | 0.00 | 0.00 |
| ATOM | 1807 | C | PRO | 218 | 1.753 | −4.126 | 15.903 | 1.00 | 0.00 | 0.396 | 9.82 | 4.00 |
| ATOM | 1808 | O | PRO | 218 | 0.795 | −4.708 | 15.372 | 1.00 | 0.00 | −0.396 | 8.17 | −17.40 |
| ATOM | 1809 | N | ASP | 219P | 1.707 | −3.654 | 17.143 | 1.00 | 0.00 | −0.650 | 9.00 | −17.40 |
| ATOM | 1810 | HN | ASP | 219P | 2.535 | −3.195 | 17.548 | 1.00 | 0.00 | 0.440 | 0.00 | 0.00 |
| ATOM | 1811 | CA | ASP | 219P | 0.495 | −3.781 | 17.929 | 1.00 | 0.00 | 0.158 | 9.40 | 4.00 |
| ATOM | 1812 | HA | ASP | 219P | −0.186 | −4.535 | 17.536 | 1.00 | 0.00 | 0.053 | 0.00 | 0.00 |
| ATOM | 1813 | CB | ASP | 219P | 0.812 | −4.288 | 19.357 | 1.00 | 0.00 | −0.336 | 12.77 | 4.00 |
| ATOM | 1814 | HB1 | ASP | 219P | 1.157 | −5.321 | 19.386 | 1.00 | 0.00 | 0.053 | 0.00 | 0.00 |
| ATOM | 1815 | HB2 | ASP | 219P | −0.044 | −4.256 | 20.031 | 1.00 | 0.00 | 0.053 | 0.00 | 0.00 |
| ATOM | 1816 | CG | ASP | 219P | 1.901 | −3.486 | 20.061 | 1.00 | 0.00 | 0.297 | 9.82 | 4.00 |
| ATOM | 1817 | OD1 | ASP | 219P | 2.355 | −2.441 | 19.545 | 1.00 | 0.00 | −0.534 | 8.17 | −18.95 |
| ATOM | 1818 | OD2 | ASP | 219P | 2.301 | −3.916 | 21.166 | 1.00 | 0.00 | −0.534 | 8.17 | −18.95 |
| ATOM | 1819 | C | ASP | 219P | −0.307 | −2.487 | 17.993 | 1.00 | 0.00 | 0.396 | 9.82 | 4.00 |
| ATOM | 1820 | O | ASP | 219P | −1.042 | −2.244 | 18.941 | 1.00 | 0.00 | −0.396 | 8.17 | −17.40 |
| ATOM | 1821 | N | PHE | 220 | −0.177 | −1.671 | 16.960 | 1.00 | 0.00 | −0.650 | 9.00 | −17.40 |
| ATOM | 1822 | HN | PHE | 220 | −0.450 | −1.937 | 16.188 | 1.00 | 0.00 | 0.440 | 0.00 | 0.00 |
| ATOM | 1823 | CA | PHE | 220 | −0.896 | −0.405 | 16.881 | 1.00 | 0.00 | 0.158 | 9.40 | 4.00 |
| ATOM | 1824 | HA | PHE | 220 | −0.632 | 0.267 | 17.697 | 1.00 | 0.00 | 0.053 | 0.00 | 0.00 |
| ATOM | 1825 | CB | PHE | 220 | −0.505 | 0.308 | 15.580 | 1.00 | 0.00 | −0.106 | 12.77 | 4.00 |
| ATOM | 1826 | HB1 | PHE | 220 | −0.671 | −0.378 | 14.750 | 1.00 | 0.00 | 0.053 | 0.00 | 0.00 |
| ATOM | 1827 | HB2 | PHE | 220 | 0.547 | 0.584 | 15.644 | 1.00 | 0.00 | 0.053 | 0.00 | 0.00 |
| ATOM | 1828 | CG | PHE | 220 | −1.294 | 1.566 | 15.301 | 1.00 | 0.00 | 0.000 | 7.26 | 0.60 |
| ATOM | 1829 | CD1 | PHE | 220 | −0.921 | 2.787 | 15.873 | 1.00 | 0.00 | −0.127 | 10.80 | 0.60 |
| ATOM | 1830 | HD1 | PHE | 220 | −0.062 | −2.826 | 16.543 | 1.00 | 0.00 | 0.127 | 0.00 | 0.00 |
| ATOM | 1831 | CD2 | PHE | 220 | −2.396 | 1.530 | 14.448 | 1.00 | 0.00 | −0.127 | 10.80 | 0.60 |
| ATOM | 1832 | HD2 | PHE | 220 | −2.697 | 0.584 | 13.998 | 1.00 | 0.00 | 0.127 | 0.00 | 0.00 |
| ATOM | 1833 | CE1 | PHE | 220 | −1.632 | 3.955 | 15.598 | 1.00 | 0.00 | −0.127 | 10.80 | 0.60 |
| ATOM | 1834 | HE1 | PHE | 220 | −1.326 | 4.897 | 16.052 | 1.00 | 0.00 | 0.127 | 0.00 | 0.00 |
| ATOM | 1835 | CE2 | PHE | 220 | −3.119 | 2.692 | 14.162 | 1.00 | 0.00 | −0.127 | 10.80 | 0.60 |
| ATOM | 1836 | HE2 | PHE | 220 | −3.978 | 2.651 | 13.492 | 1.00 | 0.00 | 0.127 | 0.00 | 0.00 |
| ATOM | 1837 | CZ | PHE | 220 | −2.730 | 3.918 | 14.745 | 1.00 | 0.00 | −0.127 | 10.80 | 0.60 |
| ATOM | 1838 | HZ | PHE | 220 | −3.286 | 4.829 | 14.528 | 1.00 | 0.00 | 0.127 | 0.00 | 0.00 |
| ATOM | 1839 | C | PHE | 220 | −2.413 | −0.600 | 16.942 | 1.00 | 0.00 | 0.396 | 9.82 | 4.00 |
| ATOM | 1840 | O | PHE | 220 | −3.085 | 0.021 | 17.761 | 1.00 | 0.00 | −0.396 | 8.17 | −17.40 |
| ATOM | 1841 | N | PHE | 221 | −2.955 | −1.452 | 16.071 | 1.00 | 0.00 | −0.650 | 9.00 | −17.40 |
| ATOM | 1842 | HN | PHE | 221 | −2.346 | −1.952 | 15.407 | 1.00 | 0.00 | 0.440 | 0.00 | 0.00 |
| ATOM | 1843 | CA | PHE | 221 | −4.393 | −1.688 | 16.042 | 1.00 | 0.00 | 0.158 | 9.40 | 4.00 |
| ATOM | 1844 | HA | PHE | 221 | −4.964 | −0.760 | 16.015 | 1.00 | 0.00 | 0.053 | 0.00 | 0.00 |
| ATOM | 1845 | CB | PHE | 221 | −4.774 | −2.465 | 14.780 | 1.00 | 0.00 | −0.106 | 12.77 | 4.00 |
| ATOM | 1846 | HB1 | PHE | 221 | −5.860 | −2.536 | 14.736 | 1.00 | 0.00 | 0.053 | 0.00 | 0.00 |
| ATOM | 1847 | HB2 | PHE | 221 | −4.326 | −3.457 | 14.839 | 1.00 | 0.00 | 0.053 | 0.00 | 0.00 |
| ATOM | 1848 | CG | PHE | 221 | −4.305 | −1.827 | 13.501 | 1.00 | 0.00 | 0.000 | 7.26 | 0.60 |
| ATOM | 1849 | CD1 | PHE | 221 | −3.001 | −2.040 | 13.035 | 1.00 | 0.00 | −0.127 | 10.80 | 0.60 |
| ATOM | 1850 | HD1 | PHE | 221 | −2.313 | −2.653 | 13.617 | 1.00 | 0.00 | 0.127 | 0.00 | 0.00 |
| ATOM | 1851 | CD2 | PHE | 221 | −5.170 | −1.039 | 12.750 | 1.00 | 0.00 | −0.127 | 10.80 | 0.60 |
| ATOM | 1852 | HD2 | PHE | 221 | −6.184 | −0.863 | 13.109 | 1.00 | 0.00 | 0.127 | 0.00 | 0.00 |
| ATOM | 1853 | CE1 | PHE | 221 | −2.567 | −1.468 | 11.817 | 1.00 | 0.00 | −0.127 | 10.80 | 0.60 |
| ATOM | 1854 | HE1 | PHE | 221 | −1.549 | −1.638 | 11.464 | 1.00 | 0.00 | 0.127 | 0.00 | 0.00 |
| ATOM | 1855 | CE2 | PHE | 221 | −4.758 | −0.466 | 11.536 | 1.00 | 0.00 | −0.127 | 10.80 | 0.60 |
| ATOM | 1856 | HE2 | PHE | 221 | −5.449 | 0.145 | 10.956 | 1.00 | 0.00 | 0.127 | 0.00 | 0.00 |
| ATOM | 1857 | CZ | PHE | 221 | −3.445 | −0.686 | 11.070 | 1.00 | 0.00 | −0.127 | 10.80 | 0.60 |
| ATOM | 1858 | HZ | PHE | 221 | −3.118 | −0.245 | 10.128 | 1.00 | 0.00 | 0.127 | 0.00 | 0.00 |
| ATOM | 1859 | C | PHE | 221 | −4.880 | −2.463 | 17.269 | 1.00 | 0.00 | 0.396 | 9.82 | 4.00 |
| ATOM | 1860 | O | PHE | 221 | −5.933 | −2.160 | 17.828 | 1.00 | 0.00 | −0.396 | 8.17 | −17.40 |
| ATOM | 1861 | N | GLU | 222 | −4.119 | −3.477 | 17.664 | 1.00 | 0.00 | −0.650 | 9.00 | −17.40 |
| ATOM | 1862 | HN | GLU | 222 | −3.267 | −3.696 | 17.127 | 1.00 | 0.00 | 0.440 | 0.00 | 0.00 |
| ATOM | 1863 | CA | GLU | 222 | −4.443 | −4.290 | 18.826 | 1.00 | 0.00 | 0.158 | 9.40 | 4.00 |
| ATOM | 1864 | HA | GLU | 222 | −5.363 | −4.823 | 18.591 | 1.00 | 0.00 | 0.053 | 0.00 | 0.00 |
| ATOM | 1865 | CB | GLU | 222 | −3.281 | −5.276 | 19.077 | 1.00 | 0.00 | −0.106 | 12.77 | 4.00 |
| ATOM | 1866 | HB1 | GLU | 222 | −2.363 | −4.694 | 19.165 | 1.00 | 0.00 | 0.053 | 0.00 | 0.00 |
| ATOM | 1867 | HB2 | GLU | 222 | −3.233 | −5.958 | 18.228 | 1.00 | 0.00 | 0.053 | 0.00 | 0.00 |
| ATOM | 1868 | CG | GLU | 222 | −3.384 | −6.123 | 20.315 | 1.00 | 0.00 | −0.106 | 12.77 | 4.00 |
| ATOM | 1869 | HG1 | GLU | 222 | −3.658 | −5.560 | 21.207 | 1.00 | 0.00 | 0.053 | 0.00 | 0.00 |
| ATOM | 1870 | HG2 | GLU | 222 | −2.455 | −6.630 | 20.575 | 1.00 | 0.00 | 0.053 | 0.00 | 0.00 |
| ATOM | 1871 | CD | GLU | 222 | −4.418 | −7.224 | 20.212 | 1.00 | 0.00 | 0.399 | 9.82 | 4.00 |
| ATOM | 1872 | OE1 | GLU | 222 | −4.788 | −7.594 | 19.078 | 1.00 | 0.00 | −0.396 | 8.17 | −18.95 |
| ATOM | 1873 | OE2 | GLU | 222 | −4.838 | −7.747 | 21.271 | 1.00 | 0.00 | −0.427 | 8.17 | −18.95 |
| ATOM | 1874 | HE2 | GLU | 222 | −5.507 | −8.496 | 21.042 | 1.00 | 0.00 | 0.424 | 0.00 | 0.00 |
| ATOM | 1875 | C | GLU | 222 | −4.622 | −3.332 | 19.999 | 1.00 | 0.00 | 0.396 | 9.82 | 4.00 |
| ATOM | 1876 | O | GLU | 222 | −5.595 | −3.403 | 20.743 | 1.00 | 0.00 | −0.396 | 8.17 | −17.40 |
| ATOM | 1877 | N | TYR | 223 | −3.693 | −2.394 | 20.123 | 1.00 | 0.00 | −0.650 | 9.00 | −17.40 |
| ATOM | 1878 | HN | TYR | 223 | −2.926 | −2.355 | 19.436 | 1.00 | 0.00 | 0.440 | 0.00 | 0.00 |
| ATOM | 1879 | CA | TYR | 223 | −3.725 | −1.419 | 21.201 | 1.00 | 0.00 | 0.158 | 9.40 | 4.00 |
| ATOM | 1680 | HA | TYR | 223 | −3.626 | −1.888 | 22.179 | 1.00 | 0.00 | 0.053 | 0.00 | 0.00 |
| ATOM | 1881 | CB | TYR | 223 | −2.538 | −0.481 | 21.034 | 1.00 | 0.00 | −0.106 | 12.77 | 4.00 |
| ATOM | 1882 | HB1 | TYR | 223 | −2.548 | −0.099 | 20.013 | 1.00 | 0.00 | 0.053 | 0.00 | 0.00 |
| ATOM | 1883 | HB2 | TYR | 223 | −1.627 | −1.049 | 21.225 | 1.00 | 0.00 | 0.053 | 0.00 | 0.00 |
| ATOM | 1884 | CG | TYR | 223 | −2.528 | 0.711 | 21.960 | 1.00 | 0.00 | 0.000 | 7.26 | 0.60 |
| ATOM | 1885 | CD1 | TYR | 223 | −2.537 | 0.542 | 23.339 | 1.00 | 0.00 | −0.127 | 10.80 | 0.60 |
| ATOM | 1886 | HD1 | TYR | 223 | −2.627 | −0.460 | 23.757 | 1.00 | 0.00 | 0.127 | 0.00 | 0.00 |
| ATOM | 1887 | CE1 | TYR | 223 | −2.431 | 1.648 | 24.203 | 1.00 | 0.00 | −0.127 | 10.80 | 0.60 |
| ATOM | 1888 | HE1 | TYR | 223 | −2.436 | 1.501 | 25.283 | 1.00 | 0.00 | 0.127 | 0.00 | 0.00 |
| ATOM | 1889 | CD2 | TYR | 223 | −2.427 | 2.010 | 21.451 | 1.00 | 0.00 | −0.127 | 10.80 | 0.60 |
| ATOM | 1890 | HD2 | TYR | 223 | −2.428 | 2.162 | 20.371 | 1.00 | 0.00 | 0.127 | 0.00 | 0.00 |
| ATOM | 1891 | CE2 | TYR | 223 | −2.324 | 3.122 | 22.303 | 1.00 | 0.00 | −0.127 | 10.80 | 0.60 |
| ATOM | 1892 | HE2 | TYR | 223 | −2.248 | 4.128 | 21.890 | 1.00 | 0.00 | 0.127 | 0.00 | 0.00 |
| ATOM | 1893 | CZ | TYR | 223 | −2.320 | 2.924 | 23.673 | 1.00 | 0.00 | 0.026 | 7.26 | 0.60 |
| ATOM | 1894 | OH | TYR | 223 | −2.161 | 3.986 | 24.528 | 1.00 | 0.00 | −0.451 | 10.94 | −17.40 |
| ATOM | 1895 | HH | TYR | 223 | −1.370 | 4.565 | 24.213 | 1.00 | 0.00 | 0.424 | 0.00 | 0.00 |
| ATOM | 1896 | C | TYR | 223 | −5.038 | −0.626 | 21.220 | 1.00 | 0.00 | 0.396 | 9.82 | 4.00 |
| ATOM | 1897 | O | TYR | 223 | −5.712 | −0.518 | 22.255 | 1.00 | 0.00 | −0.396 | 8.17 | −17.40 |
| ATOM | 1898 | N | PHE | 224 | −5.414 | −0.087 | 20.069 | 1.00 | 0.00 | −0.650 | 9.00 | −17.40 |
| ATOM | 1899 | HN | PHE | 224 | −4.837 | −0.220 | 19.226 | 1.00 | 0.00 | 0.440 | 0.00 | 0.00 |
| ATOM | 1900 | CA | PHE | 224 | −6.635 | 0.689 | 19.996 | 1.00 | 0.00 | 0.158 | 9.40 | 4.00 |
| ATOM | 1901 | HA | PHE | 224 | −6.634 | 1.402 | 20.820 | 1.00 | 0.00 | 0.053 | 0.00 | 0.00 |
| ATOM | 1902 | CB | PHE | 224 | −6.621 | 1.563 | 18.729 | 1.00 | 0.00 | −0.106 | 12.77 | 4.00 |
| ATOM | 1903 | HB1 | PHE | 224 | −7.594 | 1.995 | 18.498 | 1.00 | 0.00 | 0.053 | 0.00 | 0.00 |
| ATOM | 1904 | HB2 | PHE | 224 | −6.323 | 1.014 | 17.835 | 1.00 | 0.00 | 0.053 | 0.00 | 0.00 |
| ATOM | 1905 | CG | PHE | 224 | −5.671 | 2.728 | 18.827 | 1.00 | 0.00 | 0.000 | 7.26 | 0.60 |
| ATOM | 1906 | CD1 | PHE | 224 | −4.466 | 2.740 | 18.126 | 1.00 | 0.00 | −0.127 | 10.80 | 0.60 |
| ATOM | 1907 | HD1 | PHE | 224 | −4.225 | 1.917 | 17.453 | 1.00 | 0.00 | 0.127 | 0.00 | 0.00 |
| ATOM | 1908 | CD2 | PHE | 224 | −5.965 | 3.794 | 19.682 | 1.00 | 0.00 | −0.127 | 10.80 | 0.60 |
| ATOM | 1909 | HD2 | PHE | 224 | −6.906 | 3.796 | 20.232 | 1.00 | 0.00 | 0.127 | 0.00 | 0.00 |
| ATOM | 1910 | CE1 | PHE | 224 | −3.557 | 3.803 | 18.278 | 1.00 | 0.00 | −0.127 | 10.80 | 0.60 |
| ATOM | 1911 | HE1 | PHE | 224 | −2.616 | 3.801 | 17.727 | 1.00 | 0.00 | 0.127 | 0.00 | 0.00 |
| ATOM | 1912 | CE2 | PHE | 224 | −5.077 | 4.851 | 19.842 | 1.00 | 0.00 | −0.127 | 10.80 | 0.60 |
| ATOM | 1913 | HE2 | PHE | 224 | −5.321 | 5.673 | 20.514 | 1.00 | 0.00 | 0.127 | 0.00 | 0.00 |
| ATOM | 1914 | CZ | PHE | 224 | −3.862 | 4.857 | 19.134 | 1.00 | 0.00 | −0.127 | 10.80 | 0.60 |
| ATOM | 1915 | HZ | PHE | 224 | −3.162 | 5.683 | 19.255 | 1.00 | 0.00 | 0.127 | 0.00 | 0.00 |
| ATOM | 1916 | C | PHE | 224 | −7.926 | −0.135 | 20.099 | 1.00 | 0.00 | 0.396 | 9.82 | 4.00 |
| ATOM | 1917 | O | PHE | 224 | −8.909 | 0.370 | 20.642 | 1.00 | 0.00 | −0.396 | 8.17 | −17.40 |
| ATOM | 1918 | N | GLN | 225 | −7.955 | −1.373 | 19.597 | 1.00 | 0.00 | −0.650 | 9.00 | −17.40 |
| ATOM | 1919 | HN | GLN | 225 | −7.130 | −1.768 | 19.123 | 1.00 | 0.00 | 0.440 | 0.00 | 0.00 |
| ATOM | 1920 | CA | GLN | 225 | −9.193 | −2.160 | 19.737 | 1.00 | 0.00 | 0.158 | 9.40 | 4.00 |
| ATOM | 1921 | HA | GLN | 225 | −10.033 | −1.547 | 19.411 | 1.00 | 0.00 | 0.053 | 0.00 | 0.00 |
| ATOM | 1922 | CB | GLN | 225 | −9.187 | −3.411 | 18.842 | 1.00 | 0.00 | −0.106 | 12.77 | 4.00 |
| ATOM | 1923 | HB1 | GLN | 225 | −8.364 | −4.050 | 19.163 | 1.00 | 0.00 | 0.053 | 0.00 | 0.00 |
| ATOM | 1924 | HB2 | GLN | 225 | −9.047 | −3.085 | 17.811 | 1.00 | 0.00 | 0.053 | 0.00 | 0.00 |
| ATOM | 1925 | CG | GLN | 225 | −10.496 | −4.284 | 18.881 | 1.00 | 0.00 | −0.106 | 12.77 | 4.00 |
| ATOM | 1926 | HG1 | GLN | 225 | −10.598 | −4.692 | 19.886 | 1.00 | 0.00 | 0.053 | 0.00 | 0.00 |
| ATOM | 1927 | HG2 | GLN | 225 | −10.391 | −5.081 | 18.146 | 1.00 | 0.00 | 0.053 | 0.00 | 0.00 |
| ATOM | 1928 | CD | GLN | 225 | −11.819 | −3.523 | 18.553 | 1.00 | 0.00 | 0.396 | 9.82 | 4.00 |
| ATOM | 1929 | OE1 | GLN | 225 | −12.479 | −2.948 | 19.449 | 1.00 | 0.00 | −0.396 | 8.17 | −17.40 |
| ATOM | 1930 | NE2 | GLN | 225 | −12.202 | −3.522 | 17.268 | 1.00 | 0.00 | −0.879 | 13.25 | −17.40 |
| ATOM | 1931 | HE2 | GLN | 225 | −11.631 | −4.006 | 16.560 | 1.00 | 0.00 | 0.440 | 0.00 | 0.00 |
| ATOM | 1932 | HE2 | GLN | 225 | −13.066 | −3.036 | 16.988 | 1.00 | 0.00 | 0.440 | 0.00 | 0.00 |
| ATOM | 1933 | C | GLN | 225 | −9.360 | −2.553 | 21.211 | 1.00 | 0.00 | 0.396 | 9.82 | 4.00 |
| ATOM | 1934 | O | GLN | 225 | −10.465 | −2.742 | 21.683 | 1.00 | 0.00 | −0.396 | 8.17 | −17.40 |
| ATOM | 1935 | N | ALA | 226 | −8.264 | −2.670 | 21.953 | 1.00 | 0.00 | −0.650 | 9.00 | −17.40 |
| ATOM | 1936 | HN | ALA | 226 | −7.335 | −2.513 | 21.535 | 1.00 | 0.00 | 0.440 | 0.00 | 0.00 |
| ATOM | 1937 | CA | ALA | 226 | −8.385 | −3.023 | 23.369 | 1.00 | 0.00 | 0.158 | 9.40 | 4.00 |
| ATOM | 1938 | HA | ALA | 226 | −9.142 | −3.782 | 23.564 | 1.00 | 0.00 | 0.053 | 0.00 | 0.00 |
| ATOM | 1939 | CB | ALA | 226 | −7.088 | −3.661 | 23.872 | 1.00 | 0.00 | −0.159 | 16.15 | 4.00 |
| ATOM | 1940 | HB1 | ALA | 226 | −7.193 | −3.917 | 24.926 | 1.00 | 0.00 | 0.053 | 0.00 | 0.00 |
| ATOM | 1941 | HB2 | ALA | 226 | −6.880 | −4.563 | 23.297 | 1.00 | 0.00 | 0.053 | 0.00 | 0.00 |
| ATOM | 1942 | HB3 | ALA | 226 | −6.265 | −2.956 | 23.751 | 1.00 | 0.00 | 0.053 | 0.00 | 0.00 |
| ATOM | 1943 | C | ALA | 226 | −8.766 | −1.836 | 24.266 | 1.00 | 0.00 | 0.396 | 9.82 | 4.00 |
| ATOM | 1944 | O | ALA | 226 | −9.464 | −2.017 | 25.272 | 1.00 | 0.00 | −0.396 | 8.17 | −17.40 |
| ATOM | 1945 | N | THR | 227 | −8.324 | −0.627 | 23.915 | 1.00 | 0.00 | −0.650 | 9.00 | −17.40 |
| ATOM | 1946 | HN | THR | 227 | −7.771 | −0.519 | 23.052 | 1.00 | 0.00 | 0.440 | 0.00 | 0.00 |
| ATOM | 1947 | CA | THR | 227 | −8.610 | 0.541 | 24.732 | 1.00 | 0.00 | 0.158 | 9.40 | 4.00 |
| ATOM | 1948 | HA | THR | 227 | −8.723 | 0.212 | 25.765 | 1.00 | 0.00 | 0.053 | 0.00 | 0.00 |
| ATOM | 1949 | CB | THR | 227 | −7.401 | 1.545 | 24.747 | 1.00 | 0.00 | 0.060 | 9.40 | 4.00 |
| ATOM | 1950 | HB | THR | 227 | −7.667 | 2.443 | 25.303 | 1.00 | 0.00 | 0.053 | 0.00 | 0.00 |
| ATOM | 1951 | OG1 | THR | 227 | −7.068 | 1.954 | 23.402 | 1.00 | 0.00 | −0.537 | 11.04 | −17.40 |
| ATOM | 1952 | HG1 | THR | 227 | −6.713 | 2.921 | 23.414 | 1.00 | 0.00 | 0.424 | 0.00 | 0.00 |
| ATOM | 1953 | CG2 | THR | 227 | −6.174 | 0.888 | 25.419 | 1.00 | 0.00 | −0.159 | 16.15 | 4.00 |
| ATOM | 1954 | HG2 | THR | 227 | −5.341 | 1.591 | 25.424 | 1.00 | 0.00 | 0.053 | 0.00 | 0.00 |
| ATOM | 1955 | HG2 | THR | 227 | −6.423 | 0.613 | 26.444 | 1.00 | 0.00 | 0.053 | 0.00 | 0.00 |
| ATOM | 1956 | HG2 | THR | 227 | −5.889 | −0.005 | 24.863 | 1.00 | 0.00 | 0.053 | 0.00 | 0.00 |
| ATOM | 1957 | C | THR | 227 | −9.883 | 1.301 | 24.351 | 1.00 | 0.00 | 0.396 | 9.82 | 4.00 |
| ATOM | 1958 | O | THR | 227 | −10.380 | 2.118 | 25.137 | 1.00 | 0.00 | −0.396 | 8.17 | −17.40 |
| ATOM | 1959 | N | TYR | 228 | −10.416 | 1.035 | 23.163 | 1.00 | 0.00 | −0.650 | 9.00 | −17.40 |
| ATOM | 1960 | HN | TYR | 228 | −9.964 | 0.350 | 22.539 | 1.00 | 0.00 | 0.440 | 0.00 | 0.00 |
| ATOM | 1961 | CA | TYR | 228 | −11.642 | 1.710 | 22.737 | 1.00 | 0.00 | 0.158 | 9.40 | 4.00 |
| ATOM | 1962 | HA | TYR | 228 | −11.482 | 2.781 | 22.618 | 1.00 | 0.00 | 0.053 | 0.00 | 0.00 |
| ATOM | 1963 | CB | TYR | 228 | −12.064 | 1.177 | 21.366 | 1.00 | 0.00 | −0.106 | 12.77 | 4.00 |
| ATOM | 1964 | HB1 | TYR | 228 | −12.104 | 0.089 | 21.420 | 1.00 | 0.00 | 0.053 | 0.00 | 0.00 |
| ATOM | 1965 | HB2 | TYR | 228 | −11.324 | 1.498 | 20.632 | 1.00 | 0.00 | 0.053 | 0.00 | 0.00 |
| ATOM | 1966 | CG | TYR | 228 | −13.411 | 1.660 | 20.896 | 1.00 | 0.00 | 0.000 | 7.26 | 0.60 |
| ATOM | 1967 | CD1 | TYR | 228 | −13.645 | 3.012 | 20.653 | 1.00 | 0.00 | −0.127 | 10.80 | 0.60 |
| ATOM | 1968 | HD1 | TYR | 228 | −12.838 | 3.730 | 20.801 | 1.00 | 0.00 | 0.127 | 0.00 | 0.00 |
| ATOM | 1969 | CE1 | TYR | 228 | −14.895 | 3.463 | 20.222 | 1.00 | 0.00 | −0.127 | 10.80 | 0.60 |
| ATOM | 1970 | HE1 | TYR | 228 | −15.064 | 4.524 | 20.038 | 1.00 | 0.00 | 0.127 | 0.00 | 0.00 |
| ATOM | 1971 | CD2 | TYR | 228 | −14.461 | 0.758 | 20.695 | 1.00 | 0.00 | −0.127 | 10.80 | 0.60 |
| ATOM | 1972 | HD2 | TYR | 228 | −14.298 | −0.303 | 20.881 | 1.00 | 0.00 | 0.127 | 0.00 | 0.00 |
| ATOM | 1973 | CE2 | TYR | 228 | −15.712 | 1.193 | 20.261 | 1.00 | 0.00 | −0.127 | 10.80 | 0.60 |
| ATOM | 1974 | HE2 | TYR | 228 | −16.519 | 0.477 | 20.104 | 1.00 | 0.00 | 0.127 | 0.00 | 0.00 |
| ATOM | 1975 | CZ | TYR | 228 | −15.920 | 2.545 | 20.030 | 1.00 | 0.00 | 0.026 | 7.26 | 0.60 |
| ATOM | 1976 | OH | TYR | 228 | −17.152 | 2.981 | 19.616 | 1.00 | 0.00 | −0.451 | 10.94 | −17.40 |
| ATOM | 1977 | HH | TYR | 228 | −17.152 | 4.009 | 19.561 | 1.00 | 0.00 | 0.424 | 0.00 | 0.00 |
| ATOM | 1978 | C | TYR | 228 | −12.777 | 1.517 | 23.771 | 1.00 | 0.00 | 0.396 | 9.82 | 4.00 |
| ATOM | 1979 | O | TYR | 228 | −13.457 | 2.466 | 24.152 | 1.00 | 0.00 | −0.396 | 8.17 | −17.40 |
| ATOM | 1980 | N | PRO | 229 | −12.991 | 0.282 | 24.244 | 1.00 | 0.00 | −0.422 | 9.00 | −17.40 |
| ATOM | 1981 | CD | PRO | 229 | −12.395 | −1.014 | 23.869 | 1.00 | 0.00 | 0.105 | 12.77 | 4.00 |
| ATOM | 1982 | HD1 | PRO | 229 | −11.321 | −0.922 | 23.702 | 1.00 | 0.00 | 0.053 | 0.00 | 0.00 |
| ATOM | 1983 | HD2 | PRO | 229 | −12.838 | −1.403 | 22.952 | 1.00 | 0.00 | 0.053 | 0.00 | 0.00 |
| ATOM | 1984 | CA | PRO | 229 | −14.071 | 0.123 | 25.224 | 1.00 | 0.00 | 0.158 | 9.40 | 4.00 |
| ATOM | 1985 | HA | PRO | 229 | −14.997 | 0.479 | 24.773 | 1.00 | 0.00 | 0.053 | 0.00 | 0.00 |
| ATOM | 1986 | CB | PRO | 229 | −14.130 | −1.392 | 25.448 | 1.00 | 0.00 | −0.106 | 12.77 | 4.00 |
| ATOM | 1987 | HB1 | PRO | 229 | −14.894 | −1.849 | 24.819 | 1.00 | 0.00 | 0.053 | 0.00 | 0.00 |
| ATOM | 1988 | HB2 | PRO | 229 | −14.366 | −1.623 | 26.486 | 1.00 | 0.00 | 0.053 | 0.00 | 0.00 |
| ATOM | 1989 | CG | PRO | 229 | −12.714 | −1.873 | 25.065 | 1.00 | 0.00 | −0.106 | 12.77 | 4.00 |
| ATOM | 1990 | HG1 | PRO | 229 | −12.709 | −2.935 | 24.821 | 1.00 | 0.00 | 0.053 | 0.00 | 0.00 |
| ATOM | 1991 | HG2 | PRO | 229 | −12.009 | −1.719 | 25.882 | 1.00 | 0.00 | 0.053 | 0.00 | 0.00 |
| ATOM | 1992 | C | PRO | 229 | −13.857 | 0.901 | 26.526 | 1.00 | 0.00 | 0.396 | 9.82 | 4.00 |
| ATOM | 1993 | O | PRO | 229 | −14.828 | 1.275 | 27.185 | 1.00 | 0.00 | −0.396 | 8.17 | −17.40 |
| ATOM | 1994 | N | LEU | 230 | −12.596 | 1.151 | 26.889 | 1.00 | 0.00 | −0.650 | 9.00 | −17.40 |
| ATOM | 1995 | HN | LEU | 230 | −11.824 | 0.813 | 26.296 | 1.00 | 0.00 | 0.440 | 0.00 | 0.00 |
| ATOM | 1996 | CA | LEU | 230 | −12.288 | 1.892 | 28.108 | 1.00 | 0.00 | 0.158 | 9.40 | 4.00 |
| ATOM | 1997 | HA | LEU | 230 | −12.875 | 1.476 | 28.926 | 1.00 | 0.00 | 0.053 | 0.00 | 0.00 |
| ATOM | 1998 | CB | LEU | 230 | −10.801 | 1.781 | 28.441 | 1.00 | 0.00 | −0.106 | 12.77 | 4.00 |
| ATOM | 1999 | HB1 | LEU | 230 | −10.391 | 2.789 | 28.372 | 1.00 | 0.00 | 0.053 | 0.00 | 0.00 |
| ATOM | 2000 | HB2 | LEU | 230 | −10.367 | 1.108 | 27.701 | 1.00 | 0.00 | 0.053 | 0.00 | 0.00 |
| ATOM | 2001 | CG | LEU | 230 | −10.327 | 1.251 | 29.786 | 1.00 | 0.00 | −0.053 | 9.40 | 4.00 |
| ATOM | 2002 | HG | LEU | 230 | −10.382 | 0.163 | 29.731 | 1.00 | 0.00 | 0.053 | 0.00 | 0.00 |
| ATOM | 2003 | CD1 | LEU | 230 | −8.900 | 1.742 | 29.971 | 1.00 | 0.00 | −0.159 | 16.15 | 4.00 |
| ATOM | 2004 | HD1 | LEU | 230 | −8.513 | 1.386 | 30.926 | 1.00 | 0.00 | 0.053 | 0.00 | 0.00 |
| ATOM | 2005 | HD1 | LEU | 230 | −8.276 | 1.360 | 29.162 | 1.00 | 0.00 | 0.053 | 0.00 | 0.00 |
| ATOM | 2006 | HD1 | LEU | 230 | −8.885 | 2.831 | 29.957 | 1.00 | 0.00 | 0.053 | 0.00 | 0.00 |
| ATOM | 2007 | CD2 | LEU | 230 | −11.204 | 1.733 | 30.936 | 1.00 | 0.00 | −0.159 | 16.15 | 4.00 |
| ATOM | 2008 | HD2 | LEU | 230 | −10.826 | 1.329 | 31.875 | 1.00 | 0.00 | 0.053 | 0.00 | 0.00 |
| ATOM | 2009 | HD2 | LEU | 230 | −11.185 | 2.822 | 30.976 | 1.00 | 0.00 | 0.053 | 0.00 | 0.00 |
| ATOM | 2010 | HD2 | LEU | 230 | −12.227 | 1.393 | 30.780 | 1.00 | 0.00 | 0.053 | 0.00 | 0.00 |
| ATOM | 2011 | C | LEU | 230 | −12.649 | 3.367 | 27.890 | 1.00 | 0.00 | 0.396 | 9.82 | 4.00 |
| ATOM | 2012 | O | LEU | 230 | −13.219 | 4.023 | 28.764 | 1.00 | 0.00 | −0.396 | 8.17 | −17.40 |
| ATOM | 2013 | N | LEU | 231 | −12.317 | 3.872 | 26.711 | 1.00 | 0.00 | −0.650 | 9.00 | −17.40 |
| ATOM | 2014 | HN | LEU | 231 | −11.841 | 3.272 | 26.021 | 1.00 | 0.00 | 0.440 | 0.00 | 0.00 |
| ATOM | 2015 | CA | LEU | 231 | −12.611 | 5.259 | 26.369 | 1.00 | 0.00 | 0.158 | 9.40 | 4.00 |
| ATOM | 2016 | HA | LEU | 231 | −12.149 | 5.909 | 27.112 | 1.00 | 0.00 | 0.053 | 0.00 | 0.00 |
| ATOM | 2017 | CB | LEU | 231 | −12.037 | 5.596 | 24.988 | 1.00 | 0.00 | −0.106 | 12.77 | 4.00 |
| ATOM | 2018 | HB1 | LEU | 231 | −12.544 | 4.975 | 24.249 | 1.00 | 0.00 | 0.053 | 0.00 | 0.00 |
| ATOM | 2019 | HB2 | LEU | 231 | −10.968 | 5.382 | 25.004 | 1.00 | 0.00 | 0.053 | 0.00 | 0.00 |
| ATOM | 2020 | CG | LEU | 231 | −12.205 | 7.049 | 24.554 | 1.00 | 0.00 | −0.053 | 9.40 | 4.00 |
| ATOM | 2021 | HG | LEU | 231 | −13.261 | 7.267 | 24.400 | 1.00 | 0.00 | 0.053 | 0.00 | 0.00 |
| ATOM | 2022 | CD1 | LEU | 231 | −11.652 | 7.978 | 25.634 | 1.00 | 0.00 | −0.159 | 16.15 | 4.00 |
| ATOM | 2023 | HD1 | LEU | 231 | −11.774 | 9.014 | 25.319 | 1.00 | 0.00 | 0.053 | 0.00 | 0.00 |
| ATOM | 2024 | HD1 | LEU | 231 | −12.193 | 7.815 | 26.566 | 1.00 | 0.00 | 0.053 | 0.00 | 0.00 |
| ATOM | 2025 | HD1 | LEU | 231 | −10.593 | 7.767 | 25.787 | 1.00 | 0.00 | 0.053 | 0.00 | 0.00 |
| ATOM | 2026 | CD2 | LEU | 231 | −11.504 | 7.275 | 23.211 | 1.00 | 0.00 | −0.159 | 16.15 | 4.00 |
| ATOM | 2027 | HD2 | LEU | 231 | −11.627 | 8.314 | 22.906 | 1.00 | 0.00 | 0.053 | 0.00 | 0.00 |
| ATOM | 2028 | HD2 | LEU | 231 | −10.442 | 7.050 | 23.312 | 1.00 | 0.00 | 0.053 | 0.00 | 0.00 |
| ATOM | 2029 | HD2 | LEU | 231 | −11.942 | 6.621 | 22.457 | 1.00 | 0.00 | 0.053 | 0.00 | 0.00 |
| ATOM | 2030 | C | LEU | 231 | −14.127 | 5.455 | 26.367 | 1.00 | 0.00 | 0.396 | 9.82 | 4.00 |
| ATOM | 2031 | O | LEU | 231 | −14.638 | 6.487 | 26.815 | 1.00 | 0.00 | −0.396 | 8.17 | −17.40 |
| ATOM | 2032 | N | LYS | 232S | −14.848 | 4.454 | 25.883 | 1.00 | 0.00 | −0.650 | 9.00 | −17.40 |
| ATOM | 2033 | HN | LYS | 232S | −14.380 | 3.603 | 25.538 | 1.00 | 0.00 | 0.440 | 0.00 | 0.00 |
| ATOM | 2034 | CA | LYS | 232S | −16.304 | 4.555 | 25.837 | 1.00 | 0.00 | 0.158 | 9.40 | 4.00 |
| ATOM | 2035 | HA | LYS | 232S | −16.652 | 5.491 | 25.401 | 1.00 | 0.00 | 0.053 | 0.00 | 0.00 |
| ATOM | 2036 | CB | LYS | 232S | −16.897 | 3.465 | 24.940 | 1.00 | 0.00 | −0.106 | 12.77 | 4.00 |
| ATOM | 2037 | HB1 | LYS | 232S | −17.739 | 2.937 | 25.387 | 1.00 | 0.00 | 0.053 | 0.00 | 0.00 |
| ATOM | 2038 | HB2 | LYS | 232S | −16.188 | 2.681 | 24.672 | 1.00 | 0.00 | 0.053 | 0.00 | 0.00 |
| ATOM | 2039 | CG | LYS | 232S | −17.425 | 3.984 | 23.608 | 1.00 | 0.00 | −0.106 | 12.77 | 4.00 |
| ATOM | 2040 | HG1 | LYS | 232S | −16.635 | 4.017 | 22.857 | 1.00 | 0.00 | 0.053 | 0.00 | 0.00 |
| ATOM | 2041 | HG2 | LYS | 232S | −17.828 | 4.991 | 23.709 | 1.00 | 0.00 | 0.053 | 0.00 | 0.00 |
| ATOM | 2042 | CD | LYS | 232S | −18.536 | 3.093 | 23.071 | 1.00 | 0.00 | −0.106 | 12.77 | 4.00 |
| ATOM | 2043 | HD1 | LYS | 232S | −18.931 | 3.457 | 22.123 | 1.00 | 0.00 | 0.053 | 0.00 | 0.00 |
| ATOM | 2044 | HD2 | LYS | 232S | −19.379 | 3.029 | 23.758 | 1.00 | 0.00 | 0.053 | 0.00 | 0.00 |
| ATOM | 2045 | CE | LYS | 232S | −18.056 | 1.654 | 22.826 | 1.00 | 0.00 | 0.099 | 12.77 | 4.00 |
| ATOM | 2046 | HE1 | LYS | 232S | −17.671 | 1.185 | 23.731 | 1.00 | 0.00 | 0.053 | 0.00 | 0.00 |
| ATOM | 2047 | HE2 | LYS | 232S | −17.254 | 1.603 | 22.088 | 1.00 | 0.00 | 0.053 | 0.00 | 0.00 |
| ATOM | 2048 | NZ | LYS | 232S | −19.158 | 0.760 | 22.319 | 1.00 | 0.00 | −0.045 | 13.25 | −39.20 |
| ATOM | 2049 | HZ1 | LYS | 232S | −18.789 | −0.190 | 22.170 | 1.00 | 0.00 | 0.280 | 0.00 | 0.00 |
| ATOM | 2050 | HZ2 | LYS | 232S | −19.920 | 0.727 | 23.011 | 1.00 | 0.00 | 0.280 | 0.00 | 0.00 |
| ATOM | 2051 | HZ3 | LYS | 232S | −19.519 | 1.130 | 21.428 | 1.00 | 0.00 | 0.280 | 0.00 | 0.00 |
| ATOM | 2052 | C | LYS | 232S | −16.951 | 4.475 | 27.217 | 1.00 | 0.00 | 0.396 | 9.82 | 4.00 |
| ATOM | 2053 | O | LYS | 232S | −18.001 | 5.080 | 27.447 | 1.00 | 0.00 | −0.396 | 8.17 | −17.40 |
| ATOM | 2054 | N | ALA | 233 | −16.312 | 3.750 | 28.133 | 1.00 | 0.00 | −0.650 | 9.00 | −17.40 |
| ATOM | 2055 | HN | ALA | 233 | −15.411 | 3.313 | 27.888 | 1.00 | 0.00 | 0.440 | 0.00 | 0.00 |
| ATOM | 2056 | CA | ALA | 233 | −16.854 | 3.557 | 29.474 | 1.00 | 0.00 | 0.158 | 9.40 | 4.00 |
| ATOM | 2057 | HA | ALA | 233 | −17.940 | 3.607 | 29.399 | 1.00 | 0.00 | 0.053 | 0.00 | 0.00 |
| ATOM | 2058 | CB | ALA | 233 | −16.515 | 2.142 | 29.963 | 1.00 | 0.00 | −0.159 | 16.15 | 4.00 |
| ATOM | 2059 | HB1 | ALA | 233 | −16.919 | 1.996 | 30.964 | 1.00 | 0.00 | 0.053 | 0.00 | 0.00 |
| ATOM | 2060 | HB2 | ALA | 233 | −16.951 | 1.408 | 29.285 | 1.00 | 0.00 | 0.053 | 0.00 | 0.00 |
| ATOM | 2061 | HB3 | ALA | 233 | −15.432 | 2.014 | 29.986 | 1.00 | 0.00 | 0.053 | 0.00 | 0.00 |
| ATOM | 2062 | C | ALA | 233 | −16.448 | 4.555 | 30.542 | 1.00 | 0.00 | 0.396 | 9.82 | 4.00 |
| ATOM | 2063 | O | ALA | 233 | −17.122 | 4.675 | 31.569 | 1.00 | 0.00 | −0.396 | 8.17 | −17.40 |
| ATOM | 2064 | N | ASP | 234P | −15.355 | 5.269 | 30.328 | 1.00 | 0.00 | −0.650 | 9.00 | −17.40 |
| ATOM | 2065 | HN | ASP | 234P | −14.832 | 5.156 | 29.447 | 1.00 | 0.00 | 0.440 | 0.00 | 0.00 |
| ATOM | 2066 | CA | ASP | 234P | −14.889 | 6.210 | 31.329 | 1.00 | 0.00 | 0.158 | 9.40 | 4.00 |
| ATOM | 2067 | HA | ASP | 234P | −15.551 | 6.207 | 32.194 | 1.00 | 0.00 | 0.053 | 0.00 | 0.00 |
| ATOM | 2068 | CB | ASP | 234P | −13.499 | 5.762 | 31.788 | 1.00 | 0.00 | −0.336 | 12.77 | 4.00 |
| ATOM | 2069 | HB1 | ASP | 234P | −12.747 | 5.830 | 31.001 | 1.00 | 0.00 | 0.053 | 0.00 | 0.00 |
| ATOM | 2070 | HB2 | ASP | 234P | −13.475 | 4.726 | 32.128 | 1.00 | 0.00 | 0.053 | 0.00 | 0.00 |
| ATOM | 2071 | CG | ASP | 234P | −12.966 | 6.579 | 32.932 | 1.00 | 0.00 | 0.297 | 9.82 | 4.00 |
| ATOM | 2072 | OD1 | ASP | 234P | −13.626 | 7.560 | 33.356 | 1.00 | 0.00 | −0.534 | 8.17 | −18.95 |
| ATOM | 2073 | OD2 | ASP | 234P | −11.861 | 6.238 | 33.408 | 1.00 | 0.00 | −0.534 | 8.17 | −18.95 |
| ATOM | 2074 | C | ASP | 234P | −14.849 | 7.633 | 30.754 | 1.00 | 0.00 | 0.396 | 9.82 | 4.00 |
| ATOM | 2075 | O | ASP | 234P | −13.932 | 7.979 | 30.019 | 1.00 | 0.00 | −0.396 | 8.17 | −17.40 |
| ATOM | 2076 | N | PRO | 235 | −15.863 | 8.462 | 31.076 | 1.00 | 0.00 | −0.422 | 9.00 | −17.40 |
| ATOM | 2077 | CD | PRO | 235 | −17.026 | 8.088 | 31.906 | 1.00 | 0.00 | 0.105 | 12.77 | 4.00 |
| ATOM | 2078 | HD1 | PRO | 235 | −16.727 | 7.463 | 32.748 | 1.00 | 0.00 | 0.053 | 0.00 | 0.00 |
| ATOM | 2079 | HD2 | PRO | 235 | −17.762 | 7.530 | 31.328 | 1.00 | 0.00 | 0.053 | 0.00 | 0.00 |
| ATOM | 2080 | CA | PRO | 235 | −15.985 | 9.852 | 30.612 | 1.00 | 0.00 | 0.158 | 9.40 | 4.00 |
| ATOM | 2081 | HA | PRO | 235 | −15.923 | 9.856 | 29.523 | 1.00 | 0.00 | 0.053 | 0.00 | 0.00 |
| ATOM | 2082 | CB | PRO | 235 | −17.387 | 10.251 | 31.078 | 1.00 | 0.00 | −0.106 | 12.77 | 4.00 |
| ATOM | 2083 | HB1 | PRO | 235 | −18.138 | 9.995 | 30.331 | 1.00 | 0.00 | 0.053 | 0.00 | 0.00 |
| ATOM | 2084 | HB2 | PRO | 235 | −17.452 | 11.323 | 31.259 | 1.00 | 0.00 | 0.053 | 0.00 | 0.00 |
| ATOM | 2085 | CG | PRO | 235 | −17.552 | 9.446 | 32.353 | 1.00 | 0.00 | −0.106 | 12.77 | 4.00 |
| ATOM | 2086 | HG1 | PRO | 235 | −18.593 | 9.409 | 32.674 | 1.00 | 0.00 | 0.053 | 0.00 | 0.00 |
| ATOM | 2087 | HG2 | PRO | 235 | −16.975 | 9.871 | 33.174 | 1.00 | 0.00 | 0.053 | 0.00 | 0.00 |
| ATOM | 2088 | C | PRO | 235 | −14.902 | 10.788 | 31.141 | 1.00 | 0.00 | 0.396 | 9.82 | 4.00 |
| ATOM | 2089 | O | PRO | 235 | −14.799 | 11.940 | 30.696 | 1.00 | 0.00 | −0.396 | 8.17 | −17.40 |
| ATOM | 2090 | N | SER | 236 | −14.105 | 10.311 | 32.095 | 1.00 | 0.00 | −0.650 | 9.00 | −17.40 |
| ATOM | 2091 | HN | SER | 236 | −14.250 | 9.358 | 32.459 | 1.00 | 0.00 | 0.440 | 0.00 | 0.00 |
| ATOM | 2092 | CA | SER | 236 | −13.020 | 11.141 | 32.626 | 1.00 | 0.00 | 0.158 | 9.40 | 4.00 |
| ATOM | 2093 | HA | SER | 236 | −13.393 | 12.164 | 32.668 | 1.00 | 0.00 | 0.053 | 0.00 | 0.00 |
| ATOM | 2094 | CB | SER | 236 | −12.632 | 10.686 | 34.050 | 1.00 | 0.00 | 0.007 | 12.77 | 4.00 |
| ATOM | 2095 | HB1 | SER | 236 | −13.505 | 10.572 | 34.691 | 1.00 | 0.00 | 0.053 | 0.00 | 0.00 |
| ATOM | 2096 | HB2 | SER | 236 | −11.968 | 11.400 | 34.537 | 1.00 | 0.00 | 0.053 | 0.00 | 0.00 |
| ATOM | 2097 | OG | SER | 236 | −11.963 | 9.442 | 34.040 | 1.00 | 0.00 | −0.537 | 11.04 | −17.40 |
| ATOM | 2098 | HG | SER | 236 | −12.655 | 8.679 | 34.026 | 1.00 | 0.00 | 0.424 | 0.00 | 0.00 |
| ATOM | 2099 | C | SER | 236 | −11.831 | 11.003 | 31.653 | 1.00 | 0.00 | 0.396 | 9.82 | 4.00 |
| ATOM | 2100 | O | SER | 236 | −10.817 | 11.691 | 31.766 | 1.00 | 0.00 | −0.396 | 8.17 | −17.40 |
| ATOM | 2101 | N | LEU | 237 | −11.975 | 10.096 | 30.694 | 1.00 | 0.00 | −0.650 | 9.00 | −17.40 |
| ATOM | 2102 | HN | LEU | 237 | −12.827 | 9.518 | 30.676 | 1.00 | 0.00 | 0.440 | 0.00 | 0.00 |
| ATOM | 2103 | CA | LEU | 237 | −10.956 | 9.891 | 29.658 | 1.00 | 0.00 | 0.158 | 9.40 | 4.00 |
| ATOM | 2104 | HA | LEU | 237 | −10.044 | 10.372 | 30.010 | 1.00 | 0.00 | 0.053 | 0.00 | 0.00 |
| ATOM | 2105 | CB | LEU | 237 | −10.773 | 8.403 | 29.366 | 1.00 | 0.00 | −0.106 | 12.77 | 4.00 |
| ATOM | 2106 | HB1 | LEU | 237 | −10.259 | 8.337 | 28.407 | 1.00 | 0.00 | 0.053 | 0.00 | 0.00 |
| ATOM | 2107 | HB2 | LEU | 237 | −11.772 | 7.969 | 29.330 | 1.00 | 0.00 | 0.053 | 0.00 | 0.00 |
| ATOM | 2108 | CG | LEU | 237 | −9.979 | 7.494 | 30.306 | 1.00 | 0.00 | −0.053 | 9.40 | 4.00 |
| ATOM | 2109 | HG | LEU | 237 | −10.427 | 7.538 | 31.298 | 1.00 | 0.00 | 0.053 | 0.00 | 0.00 |
| ATOM | 2110 | CD1 | LEU | 237 | −10.031 | 6.061 | 29.766 | 1.00 | 0.00 | −0.159 | 16.15 | 4.00 |
| ATOM | 2111 | HD1 | LEU | 237 | −9.468 | 5.402 | 30.427 | 1.00 | 0.00 | 0.053 | 0.00 | 0.00 |
| ATOM | 2112 | HD1 | LEU | 237 | −11.067 | 5.727 | 29.718 | 1.00 | 0.00 | 0.053 | 0.00 | 0.00 |
| ATOM | 2113 | HD1 | LEU | 237 | −9.594 | 6.032 | 28.768 | 1.00 | 0.00 | 0.053 | 0.00 | 0.00 |
| ATOM | 2114 | CD2 | LEU | 237 | −8.537 | 8.009 | 30.421 | 1.00 | 0.00 | −0.159 | 16.15 | 4.00 |
| ATOM | 2115 | HD2 | LEU | 237 | −7.971 | 7.361 | 31.090 | 1.00 | 0.00 | 0.053 | 0.00 | 0.00 |
| ATOM | 2116 | HD2 | LEU | 237 | −8.070 | 8.007 | 29.436 | 1.00 | 0.00 | 0.053 | 0.00 | 0.00 |
| ATOM | 2117 | HD2 | LEU | 237 | −8.543 | 9.024 | 30.818 | 1.00 | 0.00 | 0.053 | 0.00 | 0.00 |
| ATOM | 2118 | C | LEU | 237 | −11.483 | 10.536 | 28.385 | 1.00 | 0.00 | 0.396 | 9.82 | 4.00 |
| ATOM | 2119 | O | LEU | 237 | −12.657 | 10.350 | 28.035 | 1.00 | 0.00 | −0.396 | 8.17 | −17.40 |
| ATOM | 2120 | N | TRP | 238 | −10.647 | 11.291 | 27.670 | 1.00 | 0.00 | −0.650 | 9.00 | −17.40 |
| ATOM | 2121 | HN | TRP | 238 | −9.681 | 11.454 | 27.987 | 1.00 | 0.00 | 0.440 | 0.00 | 0.00 |
| ATOM | 2122 | CA | TRP | 238 | −11.134 | 11.879 | 26.428 | 1.00 | 0.00 | 0.158 | 9.40 | 4.00 |
| ATOM | 2123 | HA | TRP | 238 | −12.167 | 11.615 | 26.203 | 1.00 | 0.00 | 0.053 | 0.00 | 0.00 |
| ATOM | 2124 | CB | TRP | 238 | −11.227 | 13.409 | 26.560 | 1.00 | 0.00 | −0.106 | 12.77 | 4.00 |
| ATOM | 2125 | HB1 | TRP | 238 | −11.649 | 13.629 | 27.540 | 1.00 | 0.00 | 0.053 | 0.00 | 0.00 |
| ATOM | 2126 | HB2 | TRP | 238 | −11.873 | 13.769 | 25.759 | 1.00 | 0.00 | 0.053 | 0.00 | 0.00 |
| ATOM | 2127 | CG | TRP | 238 | −9.950 | 14.186 | 26.461 | 1.00 | 0.00 | 0.000 | 7.26 | 0.60 |
| ATOM | 2128 | CD2 | TRP | 238 | −9.818 | 15.532 | 25.987 | 1.00 | 0.00 | 0.000 | 6.80 | 0.60 |
| ATOM | 2129 | CE2 | TRP | 238 | −8.458 | 15.893 | 26.116 | 1.00 | 0.00 | −0.050 | 6.80 | 0.60 |
| ATOM | 2130 | CD1 | TRP | 238 | −8.700 | 13.790 | 26.847 | 1.00 | 0.00 | −0.177 | 10.80 | 0.60 |
| ATOM | 2131 | HD1 | TRP | 238 | −8.453 | 12.811 | 27.258 | 1.00 | 0.00 | 0.127 | 0.00 | 0.00 |
| ATOM | 2132 | NE1 | TRP | 238 | −7.794 | 14.814 | 26.639 | 1.00 | 0.00 | −0.292 | 9.00 | −17.40 |
| ATOM | 2133 | HE1 | TRP | 238 | −6.785 | 14.772 | 26.843 | 1.00 | 0.00 | 0.393 | 0.00 | 0.00 |
| ATOM | 2134 | CE3 | TRP | 238 | −10.723 | 16.469 | 25.469 | 1.00 | 0.00 | −0.127 | 10.80 | 0.60 |
| ATOM | 2135 | HE3 | TRP | 238 | −11.779 | 16.221 | 25.361 | 1.00 | 0.00 | 0.127 | 0.00 | 0.00 |
| ATOM | 2136 | CZ2 | TRP | 238 | −7.978 | 17.155 | 25.742 | 1.00 | 0.00 | −0.127 | 10.80 | 0.60 |
| ATOM | 2137 | HZ2 | TRP | 238 | −6.925 | 17.415 | 25.851 | 1.00 | 0.00 | 0.127 | 0.00 | 0.00 |
| ATOM | 2138 | CZ3 | TRP | 238 | −10.245 | 17.723 | 25.094 | 1.00 | 0.00 | −0.127 | 10.80 | 0.60 |
| ATOM | 2139 | HZ3 | TRP | 238 | −10.936 | 18.460 | 24.687 | 1.00 | 0.00 | 0.127 | 0.00 | 0.00 |
| ATOM | 2140 | CH2 | TRP | 238 | −8.885 | 18.052 | 25.232 | 1.00 | 0.00 | −0.127 | 10.80 | 0.60 |
| ATOM | 2141 | HH2 | TRP | 238 | −8.542 | 19.041 | 24.927 | 1.00 | 0.00 | 0.127 | 0.00 | 0.00 |
| ATOM | 2142 | C | TRP | 238 | −10.325 | 11.449 | 25.196 | 1.00 | 0.00 | 0.396 | 9.82 | 4.00 |
| ATOM | 2143 | O | TRP | 238 | −10.612 | 11.864 | 24.069 | 1.00 | 0.00 | −0.396 | 8.17 | −17.40 |
| ATOM | 2144 | N | CYS | 239 | −9.332 | 10.588 | 25.409 | 1.00 | 0.00 | −0.650 | 9.00 | −17.40 |
| ATOM | 2145 | HN | CYS | 239 | −9.126 | 10.276 | 26.369 | 1.00 | 0.00 | 0.440 | 0.00 | 0.00 |
| ATOM | 2146 | CA | CYS | 239 | −8.531 | 10.080 | 24.298 | 1.00 | 0.00 | 0.158 | 9.40 | 4.00 |
| ATOM | 2147 | HA | CYS | 239 | −9.208 | 9.728 | 23.520 | 1.00 | 0.00 | 0.053 | 0.00 | 0.00 |
| ATOM | 2148 | C | CYS | 239 | −7.614 | 8.927 | 24.666 | 1.00 | 0.00 | 0.396 | 9.82 | 4.00 |
| ATOM | 2149 | O | CYS | 239 | −7.362 | 8.670 | 25.840 | 1.00 | 0.00 | −0.396 | 8.17 | −17.40 |
| ATOM | 2150 | CB | CYS | 239 | −7.707 | 11.227 | 23.693 | 1.00 | 0.00 | −0.041 | 12.77 | 4.00 |
| ATOM | 2151 | HB1 | CYS | 239 | −8.043 | 12.145 | 24.174 | 1.00 | 0.00 | 0.053 | 0.00 | 0.00 |
| ATOM | 2152 | HB2 | CYS | 239 | −7.909 | 11.230 | 22.622 | 1.00 | 0.00 | 0.053 | 0.00 | 0.00 |
| ATOM | 2153 | SG | CYS | 239 | −5.882 | 11.228 | 23.850 | 1.00 | 0.00 | −0.065 | 19.93 | −6.40 |
| ATOM | 2154 | N | VAL | 240 | −7.156 | 8.205 | 23.649 | 1.00 | 0.00 | −0.650 | 9.00 | −17.40 |
| ATOM | 2155 | HN | VAL | 240 | −7.491 | 8.414 | 22.698 | 1.00 | 0.00 | 0.440 | 0.00 | 0.00 |
| ATOM | 2156 | CA | VAL | 240 | −6.195 | 7.127 | 23.835 | 1.00 | 0.00 | 0.158 | 9.40 | 4.00 |
| ATOM | 2157 | HA | VAL | 240 | −5.818 | 7.109 | 24.857 | 1.00 | 0.00 | 0.053 | 0.00 | 0.00 |
| ATOM | 2158 | CB | VAL | 240 | −6.787 | 5.728 | 23.542 | 1.00 | 0.00 | −0.053 | 9.40 | 4.00 |
| ATOM | 2159 | HB | VAL | 240 | −7.201 | 5.700 | 22.534 | 1.00 | 0.00 | 0.053 | 0.00 | 0.00 |
| ATOM | 2160 | CG1 | VAL | 240 | −5.671 | 4.637 | 23.660 | 1.00 | 0.00 | −0.159 | 16.15 | 4.00 |
| ATOM | 2161 | HG1 | VAL | 240 | −6.097 | 3.655 | 23.452 | 1.00 | 0.00 | 0.053 | 0.00 | 0.00 |
| ATOM | 2162 | HG1 | VAL | 240 | −4.878 | 4.847 | 22.941 | 1.00 | 0.00 | 0.053 | 0.00 | 0.00 |
| ATOM | 2163 | HG1 | VAL | 240 | −5.258 | 4.646 | 24.668 | 1.00 | 0.00 | 0.053 | 0.00 | 0.00 |
| ATOM | 2164 | CG2 | VAL | 240 | −7.906 | 5.427 | 24.549 | 1.00 | 0.00 | −0.159 | 16.15 | 4.00 |
| ATOM | 2165 | HG2 | VAL | 240 | −8.324 | 4.441 | 24.344 | 1.00 | 0.00 | 0.053 | 0.00 | 0.00 |
| ATOM | 2166 | HG2 | VAL | 240 | −7.500 | 5.445 | 25.560 | 1.00 | 0.00 | 0.053 | 0.00 | 0.00 |
| ATOM | 2167 | HG2 | VAL | 240 | −8.689 | 6.179 | 24.458 | 1.00 | 0.00 | 0.053 | 0.00 | 0.00 |
| ATOM | 2168 | C | VAL | 240 | −5.098 | 7.465 | 22.831 | 1.00 | 0.00 | 0.396 | 9.82 | 4.00 |
| ATOM | 2169 | O | VAL | 240 | −5.346 | 7.555 | 21.619 | 1.00 | 0.00 | −0.396 | 8.17 | −17.40 |
| ATOM | 2170 | N | SER | 241 | −3.886 | 7.666 | 23.338 | 1.00 | 0.00 | −0.650 | 9.00 | −17.40 |
| ATOM | 2171 | HN | SER | 241 | −3.737 | 7.559 | 24.351 | 1.00 | 0.00 | 0.440 | 0.00 | 0.00 |
| ATOM | 2172 | CA | SER | 241 | −2.763 | 8.035 | 22.486 | 1.00 | 0.00 | 0.158 | 9.40 | 4.00 |
| ATOM | 2173 | HA | SER | 241 | −3.051 | 8.200 | 21.448 | 1.00 | 0.00 | 0.053 | 0.00 | 0.00 |
| ATOM | 2174 | CB | SER | 241 | −2.174 | 9.372 | 22.968 | 1.00 | 0.00 | 0.007 | 12.77 | 4.00 |
| ATOM | 2175 | HB1 | SER | 241 | −1.908 | 9.325 | 24.024 | 1.00 | 0.00 | 0.053 | 0.00 | 0.00 |
| ATOM | 2176 | HB2 | SER | 241 | −2.889 | 10.184 | 22.839 | 1.00 | 0.00 | 0.053 | 0.00 | 0.00 |
| ATOM | 2177 | OG | SER | 241 | −1.002 | 9.705 | 22.240 | 1.00 | 0.00 | −0.537 | 11.04 | −17.40 |
| ATOM | 2178 | HG | SER | 241 | −0.490 | 10.449 | 22.734 | 1.00 | 0.00 | 0.424 | 0.00 | 0.00 |
| ATOM | 2179 | C | SER | 241 | −1.667 | 6.970 | 22.446 | 1.00 | 0.00 | 0.396 | 9.82 | 4.00 |
| ATOM | 2180 | O | SER | 241 | −1.415 | 6.272 | 23.431 | 1.00 | 0.00 | −0.396 | 8.17 | −17.40 |
| ATOM | 2181 | N | ALA | 242 | −1.035 | 6.839 | 21.286 | 1.00 | 0.00 | −0.650 | 9.00 | −17.40 |
| ATOM | 2182 | HN | ALA | 242 | −1.322 | 7.435 | 20.496 | 1.00 | 0.00 | 0.440 | 0.00 | 0.00 |
| ATOM | 2183 | CA | ALA | 242 | 0.049 | 5.886 | 21.084 | 1.00 | 0.00 | 0.158 | 9.40 | 4.00 |
| ATOM | 2184 | HA | ALA | 242 | −0.148 | 4.950 | 21.608 | 1.00 | 0.00 | 0.053 | 0.00 | 0.00 |
| ATOM | 2185 | CB | ALA | 242 | 0.200 | 5.606 | 19.596 | 1.00 | 0.00 | −0.159 | 16.15 | 4.00 |
| ATOM | 2186 | HB1 | ALA | 242 | 1.010 | 4.893 | 19.440 | 1.00 | 0.00 | 0.053 | 0.00 | 0.00 |
| ATOM | 2187 | HB2 | ALA | 242 | −0.729 | 5.189 | 19.208 | 1.00 | 0.00 | 0.053 | 0.00 | 0.00 |
| ATOM | 2188 | HB3 | ALA | 242 | 0.427 | 6.534 | 19.072 | 1.00 | 0.00 | 0.053 | 0.00 | 0.00 |
| ATOM | 2189 | C | ALA | 242 | 1.372 | 6.442 | 21.603 | 1.00 | 0.00 | 0.396 | 9.82 | 4.00 |
| ATOM | 2190 | O | ALA | 242 | 2.348 | 5.707 | 21.782 | 1.00 | 0.00 | −0.396 | 8.17 | −17.40 |
| ATOM | 2191 | N | TRP | 243 | 1.383 | 7.735 | 21.882 | 1.00 | 0.00 | −0.650 | 9.00 | −17.40 |
| ATOM | 2192 | HN | TRP | 243 | 0.498 | 8.260 | 21.839 | 1.00 | 0.00 | 0.440 | 0.00 | 0.00 |
| ATOM | 2193 | CA | TRP | 243 | 2.607 | 8.442 | 22.249 | 1.00 | 0.00 | 0.158 | 9.40 | 4.00 |
| ATOM | 2194 | HA | TRP | 243 | 3.428 | 7.891 | 21.790 | 1.00 | 0.00 | 0.053 | 0.00 | 0.00 |
| ATOM | 2195 | CB | TRP | 243 | 2.547 | 9.806 | 21.533 | 1.00 | 0.00 | −0.106 | 12.77 | 4.00 |
| ATOM | 2196 | HB1 | TRP | 243 | 1.857 | 10.441 | 22.088 | 1.00 | 0.00 | 0.053 | 0.00 | 0.00 |
| ATOM | 2197 | HB2 | TRP | 243 | 2.191 | 9.631 | 20.517 | 1.00 | 0.00 | 0.053 | 0.00 | 0.00 |
| ATOM | 2198 | CG | TRP | 243 | 3.842 | 10.566 | 21.416 | 1.00 | 0.00 | 0.000 | 7.26 | 0.60 |
| ATOM | 2199 | CD2 | TRP | 243 | 4.825 | 10.422 | 20.378 | 1.00 | 0.00 | 0.000 | 6.80 | 0.60 |
| ATOM | 2200 | CE2 | TRP | 243 | 5.872 | 11.332 | 20.659 | 1.00 | 0.00 | −0.050 | 6.80 | 0.60 |
| ATOM | 2201 | CD1 | TRP | 243 | 4.317 | 11.529 | 22.265 | 1.00 | 0.00 | −0.177 | 10.80 | 0.60 |
| ATOM | 2202 | HD1 | TRP | 243 | 3.807 | 11.876 | 23.163 | 1.00 | 0.00 | 0.127 | 0.00 | 0.00 |
| ATOM | 2203 | NE1 | TRP | 243 | 5.543 | 11.998 | 21.813 | 1.00 | 0.00 | −0.292 | 9.00 | −17.40 |
| ATOM | 2204 | HE1 | TRP | 243 | 6.111 | 12.725 | 22.268 | 1.00 | 0.00 | 0.393 | 0.00 | 0.00 |
| ATOM | 2205 | CE3 | TRP | 243 | 4.917 | 9.615 | 19.234 | 1.00 | 0.00 | −0.127 | 10.80 | 0.60 |
| ATOM | 2206 | HE3 | TRP | 243 | 4.127 | 8.904 | 18.990 | 1.00 | 0.00 | 0.127 | 0.00 | 0.00 |
| ATOM | 2207 | CZ2 | TRP | 243 | 6.998 | 11.455 | 19.839 | 1.00 | 0.00 | −0.127 | 10.80 | 0.60 |
| ATOM | 2208 | HZ2 | TRP | 243 | 7.793 | 12.161 | 20.075 | 1.00 | 0.00 | 0.127 | 0.00 | 0.00 |
| ATOM | 2209 | CZ3 | TRP | 243 | 6.043 | 9.741 | 18.412 | 1.00 | 0.00 | −0.127 | 10.80 | 0.60 |
| ATOM | 2210 | HZ3 | TRP | 243 | 6.127 | 9.123 | 17.517 | 1.00 | 0.00 | 0.127 | 0.00 | 0.00 |
| ATOM | 2211 | CH2 | TRP | 243 | 7.067 | 10.654 | 18.725 | 1.00 | 0.00 | −0.127 | 10.80 | 0.60 |
| ATOM | 2212 | HH2 | TRP | 243 | 7.934 | 10.727 | 18.069 | 1.00 | 0.00 | 0.127 | 0.00 | 0.00 |
| ATOM | 2213 | C | TRP | 243 | 3.031 | 8.636 | 23.709 | 1.00 | 0.00 | 0.396 | 9.82 | 4.00 |
| ATOM | 2214 | O | TRP | 243 | 2.241 | 9.068 | 24.560 | 1.00 | 0.00 | −0.396 | 8.17 | −17.40 |
| ATOM | 2215 | N | ASN | 244 | 4.293 | 8.295 | 23.993 | 1.00 | 0.00 | −0.650 | 9.00 | −17.40 |
| ATOM | 2216 | HN | ASN | 244 | 4.879 | 7.863 | 23.264 | 1.00 | 0.00 | 0.440 | 0.00 | 0.00 |
| ATOM | 2217 | CA | ASN | 244 | 4.853 | 8.528 | 25.328 | 1.00 | 0.00 | 0.158 | 9.40 | 4.00 |
| ATOM | 2218 | HA | ASN | 244 | 4.068 | 8.770 | 26.044 | 1.00 | 0.00 | 0.053 | 0.00 | 0.00 |
| ATOM | 2219 | CB | ASN | 244 | 5.631 | 7.304 | 25.846 | 1.00 | 0.00 | −0.106 | 12.77 | 4.00 |
| ATOM | 2220 | HB1 | ASN | 244 | 6.383 | 6.965 | 25.134 | 1.00 | 0.00 | 0.053 | 0.00 | 0.00 |
| ATOM | 2221 | HB2 | ASN | 244 | 4.976 | 6.454 | 26.039 | 1.00 | 0.00 | 0.053 | 0.00 | 0.00 |
| ATOM | 2222 | CG | ASN | 244 | 6.371 | 7.603 | 27.165 | 1.00 | 0.00 | 0.396 | 9.82 | 4.00 |
| ATOM | 2223 | OD1 | ASN | 244 | 6.322 | 8.732 | 27.667 | 1.00 | 0.00 | −0.396 | 8.17 | −17.40 |
| ATOM | 2224 | ND2 | ASN | 244 | 7.056 | 6.605 | 27.717 | 1.00 | 0.00 | −0.879 | 13.25 | −17.40 |
| ATOM | 2225 | HD2 | ASN | 244 | 7.070 | 5.679 | 27.266 | 1.00 | 0.00 | 0.440 | 0.00 | 0.00 |
| ATOM | 2226 | HD2 | ASN | 244 | 7.571 | 6.758 | 28.595 | 1.00 | 0.00 | 0.440 | 0.00 | 0.00 |
| ATOM | 2227 | C | ASN | 244 | 5.811 | 9.709 | 25.112 | 1.00 | 0.00 | 0.396 | 9.82 | 4.00 |
| ATOM | 2228 | O | ASN | 244 | 6.852 | 9.561 | 24.464 | 1.00 | 0.00 | −0.396 | 8.17 | −17.40 |
| ATOM | 2229 | N | ASP | 245P | 5.462 | 10.886 | 25.621 | 1.00 | 0.00 | −0.650 | 9.00 | −17.40 |
| ATOM | 2230 | HN | ASP | 245P | 4.590 | 10.966 | 26.164 | 1.00 | 0.00 | 0.440 | 0.00 | 0.00 |
| ATOM | 2231 | CA | ASP | 245P | 6.302 | 12.073 | 25.419 | 1.00 | 0.00 | 0.158 | 9.40 | 4.00 |
| ATOM | 2232 | HA | ASP | 245P | 6.322 | 12.290 | 24.351 | 1.00 | 0.00 | 0.053 | 0.00 | 0.00 |
| ATOM | 2233 | CB | ASP | 245P | 5.640 | 13.286 | 26.072 | 1.00 | 0.00 | −0.336 | 12.77 | 4.00 |
| ATOM | 2234 | HB1 | ASP | 245P | 6.340 | 14.109 | 26.212 | 1.00 | 0.00 | 0.053 | 0.00 | 0.00 |
| ATOM | 2235 | HB2 | ASP | 245P | 5.231 | 13.050 | 27.054 | 1.00 | 0.00 | 0.053 | 0.00 | 0.00 |
| ATOM | 2236 | CG | ASP | 245P | 4.496 | 13.825 | 25.243 | 1.00 | 0.00 | 0.297 | 9.82 | 4.00 |
| ATOM | 2237 | OD1 | ASP | 245P | 4.764 | 14.540 | 24.250 | 1.00 | 0.00 | −0.534 | 8.17 | −18.95 |
| ATOM | 2238 | OD2 | ASP | 245P | 3.326 | 13.522 | 25.566 | 1.00 | 0.00 | −0.534 | 8.17 | −18.95 |
| ATOM | 2239 | C | ASP | 245P | 7.759 | 11.953 | 25.881 | 1.00 | 0.00 | 0.396 | 9.82 | 4.00 |
| ATOM | 2240 | O | ASP | 245P | 8.640 | 12.658 | 25.368 | 1.00 | 0.00 | −0.396 | 8.17 | −17.40 |
| ATOM | 2241 | N | ASN | 246 | 8.005 | 11.068 | 26.848 | 1.00 | 0.00 | −0.650 | 9.00 | −17.40 |
| ATOM | 2242 | HN | ASN | 246 | 7.218 | 10.532 | 27.242 | 1.00 | 0.00 | 0.440 | 0.00 | 0.00 |
| ATOM | 2243 | CA | ASN | 246 | 9.346 | 10.830 | 27.368 | 1.00 | 0.00 | 0.158 | 9.40 | 4.00 |
| ATOM | 2244 | HA | ASN | 246 | 10.031 | 11.553 | 26.927 | 1.00 | 0.00 | 0.053 | 0.00 | 0.00 |
| ATOM | 2245 | CB | ASN | 246 | 9.352 | 10.992 | 28.891 | 1.00 | 0.00 | −0.106 | 12.77 | 4.00 |
| ATOM | 2246 | HB1 | ASN | 246 | 10.307 | 10.715 | 29.336 | 1.00 | 0.00 | 0.053 | 0.00 | 0.00 |
| ATOM | 2247 | HB2 | ASN | 246 | 8.597 | 10.375 | 29.379 | 1.00 | 0.00 | 0.053 | 0.00 | 0.00 |
| ATOM | 2248 | CG | ASN | 246 | 9.079 | 12.418 | 29.324 | 1.00 | 0.00 | 0.396 | 9.82 | 4.00 |
| ATOM | 2249 | OD1 | ASN | 246 | 9.828 | 13.339 | 28.973 | 1.00 | 0.00 | −0.396 | 8.17 | −17.40 |
| ATOM | 2250 | ND2 | ASN | 246 | 8.003 | 12.615 | 30.081 | 1.00 | 0.00 | −0.879 | 13.25 | −17.40 |
| ATOM | 2251 | HD2 | ASN | 246 | 7.408 | 11.817 | 30.348 | 1.00 | 0.00 | 0.440 | 0.00 | 0.00 |
| ATOM | 2252 | HD2 | ASN | 246 | 7.764 | 13.565 | 30.399 | 1.00 | 0.00 | 0.440 | 0.00 | 0.00 |
| ATOM | 2253 | C | ASN | 246 | 9.781 | 9.407 | 26.998 | 1.00 | 0.00 | 0.396 | 9.82 | 4.00 |
| ATOM | 2254 | O | ASN | 246 | 10.603 | 8.793 | 27.678 | 1.00 | 0.00 | −0.396 | 8.17 | −17.40 |
| ATOM | 2255 | N | GLY | 247 | 9.233 | 8.896 | 25.900 | 1.00 | 0.00 | −0.650 | 9.00 | −17.40 |
| ATOM | 2256 | HN | GLY | 247 | 8.583 | 9.468 | 25.341 | 1.00 | 0.00 | 0.440 | 0.00 | 0.00 |
| ATOM | 2257 | CA | GLY | 247 | 9.539 | 7.543 | 25.480 | 1.00 | 0.00 | 0.105 | 9.40 | 4.00 |
| ATOM | 2258 | HA1 | GLY | 247 | 8.755 | 7.241 | 24.784 | 1.00 | 0.00 | 0.053 | 0.00 | 0.00 |
| ATOM | 2259 | HA2 | GLY | 247 | 9.545 | 6.925 | 26.378 | 1.00 | 0.00 | 0.053 | 0.00 | 0.00 |
| ATOM | 2260 | C | GLY | 247 | 10.854 | 7.253 | 24.771 | 1.00 | 0.00 | 0.396 | 9.82 | 4.00 |
| ATOM | 2261 | O | GLY | 247 | 10.866 | 6.480 | 23.805 | 1.00 | 0.00 | −0.396 | 8.17 | −17.40 |
| ATOM | 2262 | N | LYS | 248S | 11.950 | 7.859 | 25.225 | 1.00 | 0.00 | −0.650 | 9.00 | −17.40 |
| ATOM | 2263 | HN | LYS | 248S | 11.871 | 8.516 | 26.014 | 1.00 | 0.00 | 0.440 | 0.00 | 0.00 |
| ATOM | 2264 | CA | LYS | 248S | 13.261 | 7.613 | 24.629 | 1.00 | 0.00 | 0.158 | 9.40 | 4.00 |
| ATOM | 2265 | HA | LYS | 248S | 13.151 | 7.684 | 23.547 | 1.00 | 0.00 | 0.053 | 0.00 | 0.00 |
| ATOM | 2266 | CB | LYS | 248S | 14.268 | 8.657 | 25.116 | 1.00 | 0.00 | −0.106 | 12.77 | 4.00 |
| ATOM | 2267 | HB1 | LYS | 248S | 15.222 | 8.466 | 24.625 | 1.00 | 0.00 | 0.053 | 0.00 | 0.00 |
| ATOM | 2268 | HB2 | LYS | 248S | 14.365 | 8.559 | 26.197 | 1.00 | 0.00 | 0.053 | 0.00 | 0.00 |
| ATOM | 2269 | CG | LYS | 248S | 13.874 | 10.104 | 24.815 | 1.00 | 0.00 | −0.106 | 12.77 | 4.00 |
| ATOM | 2270 | HG1 | LYS | 248S | 12.915 | 10.317 | 25.288 | 1.00 | 0.00 | 0.053 | 0.00 | 0.00 |
| ATOM | 2271 | HG2 | LYS | 248S | 13.791 | 10.232 | 23.735 | 1.00 | 0.00 | 0.053 | 0.00 | 0.00 |
| ATOM | 2272 | CD | LYS | 248S | 14.945 | 11.036 | 25.369 | 1.00 | 0.00 | −0.106 | 12.77 | 4.00 |
| ATOM | 2273 | HD1 | LYS | 248S | 15.927 | 10.871 | 24.925 | 1.00 | 0.00 | 0.053 | 0.00 | 0.00 |
| ATOM | 2274 | HD2 | LYS | 248S | 15.088 | 10.932 | 26.444 | 1.00 | 0.00 | 0.053 | 0.00 | 0.00 |
| ATOM | 2275 | CE | LYS | 248S | 14.636 | 12.501 | 25.140 | 1.00 | 0.00 | 0.099 | 12.77 | 4.00 |
| ATOM | 2276 | HE1 | LYS | 248S | 13.672 | 12.743 | 25.587 | 1.00 | 0.00 | 0.053 | 0.00 | 0.00 |
| ATOM | 2277 | HE2 | LYS | 248S | 14.600 | 12.701 | 24.069 | 1.00 | 0.00 | 0.053 | 0.00 | 0.00 |
| ATOM | 2278 | NZ | LYS | 248S | 15.711 | 13.346 | 25.774 | 1.00 | 0.00 | −0.045 | 13.25 | −39.20 |
| ATOM | 2279 | HZ1 | LYS | 248S | 15.501 | 14.342 | 25.618 | 1.00 | 0.00 | 0.280 | 0.00 | 0.00 |
| ATOM | 2280 | HZ2 | LYS | 248S | 15.745 | 13.156 | 26.786 | 1.00 | 0.00 | 0.280 | 0.00 | 0.00 |
| ATOM | 2281 | HZ3 | LYS | 248S | 16.622 | 13.117 | 25.351 | 1.00 | 0.00 | 0.280 | 0.00 | 0.00 |
| ATOM | 2282 | C | LYS | 248S | 13.708 | 6.208 | 25.049 | 1.00 | 0.00 | 0.396 | 9.82 | 4.00 |
| ATOM | 2283 | O | LYS | 248S | 13.255 | 5.698 | 26.075 | 1.00 | 0.00 | −0.396 | 8.17 | −17.40 |
| ATOM | 2284 | N | GLU | 249 | 14.601 | 5.588 | 24.280 | 1.00 | 0.00 | −0.650 | 9.00 | −17.40 |
| ATOM | 2285 | HN | GLU | 249 | 14.995 | 6.078 | 23.464 | 1.00 | 0.00 | 0.440 | 0.00 | 0.00 |
| ATOM | 2286 | CA | GLU | 249 | 15.030 | 4.221 | 24.576 | 1.00 | 0.00 | 0.158 | 9.40 | 4.00 |
| ATOM | 2287 | HA | GLU | 249 | 14.178 | 3.563 | 24.402 | 1.00 | 0.00 | 0.053 | 0.00 | 0.00 |
| ATOM | 2288 | CB | GLU | 249 | 16.104 | 3.755 | 23.577 | 1.00 | 0.00 | −0.106 | 12.77 | 4.00 |
| ATOM | 2289 | HB1 | GLU | 249 | 16.995 | 4.365 | 23.721 | 1.00 | 0.00 | 0.053 | 0.00 | 0.00 |
| ATOM | 2290 | HB2 | GLU | 249 | 15.712 | 3.882 | 22.567 | 1.00 | 0.00 | 0.053 | 0.00 | 0.00 |
| ATOM | 2291 | CG | GLU | 249 | 16.516 | 2.266 | 23.742 | 1.00 | 0.00 | −0.106 | 12.77 | 4.00 |
| ATOM | 2292 | HG1 | GLU | 249 | 15.701 | 1.569 | 23.542 | 1.00 | 0.00 | 0.053 | 0.00 | 0.00 |
| ATOM | 2293 | HG2 | GLU | 249 | 16.864 | 2.024 | 24.746 | 1.00 | 0.00 | 0.053 | 0.00 | 0.00 |
| ATOM | 2294 | CD | GLU | 249 | 17.659 | 1.842 | 22.798 | 1.00 | 0.00 | 0.399 | 9.82 | 4.00 |
| ATOM | 2295 | OE1 | GLU | 249 | 17.425 | 1.664 | 21.581 | 1.00 | 0.00 | −0.396 | 8.17 | −18.95 |
| ATOM | 2296 | OE2 | GLU | 249 | 18.804 | 1.697 | 23.276 | 1.00 | 0.00 | −0.427 | 8.17 | −18.95 |
| ATOM | 2297 | HE2 | GLU | 249 | 19.457 | 1.424 | 22.527 | 1.00 | 0.00 | 0.424 | 0.00 | 0.00 |
| ATOM | 2298 | C | GLU | 249 | 15.519 | 3.940 | 25.993 | 1.00 | 0.00 | 0.396 | 9.82 | 4.00 |
| ATOM | 2299 | O | GLU | 249 | 15.109 | 2.958 | 26.593 | 1.00 | 0.00 | −0.396 | 8.17 | −17.40 |
| ATOM | 2300 | N | GLN | 250 | 16.398 | 4.776 | 26.531 | 1.00 | 0.00 | −0.650 | 9.00 | −17.40 |
| ATOM | 2301 | HN | GLN | 250 | 16.715 | 5.601 | 26.002 | 1.00 | 0.00 | 0.440 | 0.00 | 0.00 |
| ATOM | 2302 | CA | GLN | 250 | 16.913 | 4.524 | 27.871 | 1.00 | 0.00 | 0.158 | 9.40 | 4.00 |
| ATOM | 2303 | HA | GLN | 250 | 17.095 | 3.470 | 28.081 | 1.00 | 0.00 | 0.053 | 0.00 | 0.00 |
| ATOM | 2304 | CB | GLN | 250 | 18.293 | 5.177 | 28.048 | 1.00 | 0.00 | −0.106 | 12.77 | 4.00 |
| ATOM | 2305 | HB1 | GLN | 250 | 18.605 | 5.034 | 29.082 | 1.00 | 0.00 | 0.053 | 0.00 | 0.00 |
| ATOM | 2306 | HB2 | GLN | 250 | 18.198 | 6.237 | 27.815 | 1.00 | 0.00 | 0.053 | 0.00 | 0.00 |
| ATOM | 2307 | CG | GLN | 250 | 19.398 | 4.607 | 27.152 | 1.00 | 0.00 | −0.106 | 12.77 | 4.00 |
| ATOM | 2308 | HG1 | GLN | 250 | 20.383 | 5.025 | 27.355 | 1.00 | 0.00 | 0.053 | 0.00 | 0.00 |
| ATOM | 2309 | HG2 | GLN | 250 | 19.230 | 4.780 | 26.089 | 1.00 | 0.00 | 0.053 | 0.00 | 0.00 |
| ATOM | 2310 | CD | GLN | 250 | 19.574 | 3.093 | 27.285 | 1.00 | 0.00 | 0.396 | 9.82 | 4.00 |
| ATOM | 2311 | OE1 | GLN | 250 | 19.674 | 2.550 | 28.396 | 1.00 | 0.00 | −0.396 | 8.17 | −17.40 |
| ATOM | 2312 | NE2 | GLN | 250 | 19.625 | 2.406 | 26.148 | 1.00 | 0.00 | −0.879 | 13.25 | −17.40 |
| ATOM | 2313 | HE2 | GLN | 250 | 19.537 | 2.895 | 25.246 | 1.00 | 0.00 | 0.440 | 0.00 | 0.00 |
| ATOM | 2314 | HE2 | GLN | 250 | 19.751 | 1.384 | 26.169 | 1.00 | 0.00 | 0.440 | 0.00 | 0.00 |
| ATOM | 2315 | C | GLN | 250 | 15.979 | 4.990 | 28.990 | 1.00 | 0.00 | 0.396 | 9.82 | 4.00 |
| ATOM | 2316 | O | GLN | 250 | 16.310 | 4.860 | 30.172 | 1.00 | 0.00 | −0.396 | 8.17 | −17.40 |
| ATOM | 2317 | N | MET | 251 | 14.810 | 5.515 | 28.619 | 1.00 | 0.00 | −0.650 | 9.00 | −17.40 |
| ATOM | 2318 | HN | MET | 251 | 14.582 | 5.579 | 27.616 | 1.00 | 0.00 | 0.440 | 0.00 | 0.00 |
| ATOM | 2319 | CA | MET | 251 | 13.851 | 5.997 | 29.596 | 1.00 | 0.00 | 0.158 | 9.40 | 4.00 |
| ATOM | 2320 | HA | MET | 251 | 14.329 | 5.980 | 30.575 | 1.00 | 0.00 | 0.053 | 0.00 | 0.00 |
| ATOM | 2321 | CB | MET | 251 | 13.527 | 7.464 | 29.321 | 1.00 | 0.00 | −0.106 | 12.77 | 4.00 |
| ATOM | 2322 | HB1 | MET | 251 | 12.678 | 7.806 | 29.913 | 1.00 | 0.00 | 0.053 | 0.00 | 0.00 |
| ATOM | 2323 | HB2 | MET | 251 | 13.276 | 7.631 | 28.273 | 1.00 | 0.00 | 0.053 | 0.00 | 0.00 |
| ATOM | 2324 | CG | MET | 251 | 14.722 | 8.385 | 29.658 | 1.00 | 0.00 | −0.041 | 12.77 | 4.00 |
| ATOM | 2325 | HG1 | MET | 251 | 15.568 | 8.089 | 29.038 | 1.00 | 0.00 | 0.053 | 0.00 | 0.00 |
| ATOM | 2326 | HG2 | MET | 251 | 14.960 | 8.264 | 30.714 | 1.00 | 0.00 | 0.053 | 0.00 | 0.00 |
| ATOM | 2327 | SD | MET | 251 | 14.415 | 10.108 | 29.367 | 1.00 | 0.00 | −0.130 | 16.39 | −6.40 |
| ATOM | 2328 | CE | MET | 251 | 13.476 | 10.468 | 30.775 | 1.00 | 0.00 | −0.094 | 16.15 | 4.00 |
| ATOM | 2329 | HE1 | MET | 251 | 13.190 | 11.519 | 30.762 | 1.00 | 0.00 | 0.053 | 0.00 | 0.00 |
| ATOM | 2330 | HE2 | MET | 251 | 12.579 | 9.848 | 30.782 | 1.00 | 0.00 | 0.053 | 0.00 | 0.00 |
| ATOM | 2331 | HE3 | MET | 251 | 14.066 | 10.262 | 31.668 | 1.00 | 0.00 | 0.053 | 0.00 | 0.00 |
| ATOM | 2332 | C | MET | 251 | 12.566 | 5.184 | 29.706 | 1.00 | 0.00 | 0.396 | 9.82 | 4.00 |
| ATOM | 2333 | O | MET | 251 | 11.623 | 5.608 | 30.378 | 1.00 | 0.00 | −0.396 | 8.17 | −17.40 |
| ATOM | 2334 | N | VAL | 252 | 12.518 | 4.036 | 29.034 | 1.00 | 0.00 | −0.650 | 9.00 | −17.40 |
| ATOM | 2335 | HN | VAL | 252 | 13.323 | 3.754 | 28.457 | 1.00 | 0.00 | 0.440 | 0.00 | 0.00 |
| ATOM | 2336 | CA | VAL | 252 | 11.346 | 3.167 | 29.098 | 1.00 | 0.00 | 0.158 | 9.40 | 4.00 |
| ATOM | 2337 | HA | VAL | 252 | 10.677 | 3.561 | 29.862 | 1.00 | 0.00 | 0.053 | 0.00 | 0.00 |
| ATOM | 2338 | CB | VAL | 252 | 10.556 | 3.105 | 27.742 | 1.00 | 0.00 | −0.053 | 9.40 | 4.00 |
| ATOM | 2339 | HB | VAL | 252 | 9.651 | 2.508 | 27.857 | 1.00 | 0.00 | 0.053 | 0.00 | 0.00 |
| ATOM | 2340 | CG1 | VAL | 252 | 10.159 | 4.509 | 27.295 | 1.00 | 0.00 | −0.159 | 16.15 | 4.00 |
| ATOM | 2341 | HG1 | VAL | 252 | 9.612 | 4.450 | 26.354 | 1.00 | 0.00 | 0.053 | 0.00 | 0.00 |
| ATOM | 2342 | HG1 | VAL | 252 | 9.525 | 4.967 | 28.054 | 1.00 | 0.00 | 0.053 | 0.00 | 0.00 |
| ATOM | 2343 | HG1 | VAL | 252 | 11.055 | 5.113 | 27.156 | 1.00 | 0.00 | 0.053 | 0.00 | 0.00 |
| ATOM | 2344 | CG2 | VAL | 252 | 11.403 | 2.430 | 26.653 | 1.00 | 0.00 | −0.159 | 16.15 | 4.00 |
| ATOM | 2345 | HG2 | VAL | 252 | 10.838 | 2.396 | 25.721 | 1.00 | 0.00 | 0.053 | 0.00 | 0.00 |
| ATOM | 2346 | HG2 | VAL | 252 | 12.320 | 2.998 | 26.501 | 1.00 | 0.00 | 0.053 | 0.00 | 0.00 |
| ATOM | 2347 | HG2 | VAL | 252 | 11.652 | 1.415 | 26.962 | 1.00 | 0.00 | 0.053 | 0.00 | 0.00 |
| ATOM | 2348 | C | VAL | 252 | 11.823 | 1.760 | 29.461 | 1.00 | 0.00 | 0.396 | 9.82 | 4.00 |
| ATOM | 2349 | O | VAL | 252 | 12.972 | 1.388 | 29.156 | 1.00 | 0.00 | −0.396 | 8.17 | −17.40 |
| ATOM | 2350 | N | ASP | 253P | 10.942 | 0.996 | 30.111 | 1.00 | 0.00 | −0.650 | 9.00 | −17.40 |
| ATOM | 2351 | HN | ASP | 253P | 10.019 | 1.398 | 30.327 | 1.00 | 0.00 | 0.440 | 0.00 | 0.00 |
| ATOM | 2352 | CA | ASP | 253P | 11.211 | −0.386 | 30.536 | 1.00 | 0.00 | 0.158 | 9.40 | 4.00 |
| ATOM | 2353 | HA | ASP | 253P | 12.275 | −0.511 | 30.736 | 1.00 | 0.00 | 0.053 | 0.00 | 0.00 |
| ATOM | 2354 | CB | ASP | 253P | 10.422 | −0.693 | 31.816 | 1.00 | 0.00 | −0.336 | 12.77 | 4.00 |
| ATOM | 2355 | HB1 | ASP | 253P | 9.345 | −0.739 | 31.650 | 1.00 | 0.00 | 0.053 | 0.00 | 0.00 |
| ATOM | 2356 | HB2 | ASP | 253P | 10.570 | 0.054 | 32.595 | 1.00 | 0.00 | 0.053 | 0.00 | 0.00 |
| ATOM | 2357 | CG | ASP | 253P | 10.804 | −2.027 | 32.442 | 1.00 | 0.00 | 0.297 | 9.82 | 4.00 |
| ATOM | 2358 | OD1 | ASP | 253P | 11.407 | −2.881 | 31.753 | 1.00 | 0.00 | −0.534 | 8.17 | −18.95 |
| ATOM | 2359 | OD2 | ASP | 253P | 10.487 | −2.230 | 33.635 | 1.00 | 0.00 | −0.534 | 8.17 | −18.95 |
| ATOM | 2360 | C | ASP | 253P | 10.790 | −1.363 | 29.429 | 1.00 | 0.00 | 0.396 | 9.82 | 4.00 |
| ATOM | 2361 | O | ASP | 253P | 9.592 | −1.666 | 29.263 | 1.00 | 0.00 | −0.396 | 8.17 | −17.40 |
| ATOM | 2362 | N | SER | 254 | 11.773 | −1.870 | 28.688 | 1.00 | 0.00 | −0.650 | 9.00 | −17.40 |
| ATOM | 2363 | HN | SER | 254 | 12.746 | −1.604 | 28.894 | 1.00 | 0.00 | 0.440 | 0.00 | 0.00 |
| ATOM | 2364 | CA | SER | 254 | 11.505 | −2.794 | 27.589 | 1.00 | 0.00 | 0.158 | 9.40 | 4.00 |
| ATOM | 2365 | HA | SER | 254 | 10.800 | −2.318 | 26.906 | 1.00 | 0.00 | 0.053 | 0.00 | 0.00 |
| ATOM | 2366 | CB | SER | 254 | 12.791 | −3.050 | 26.796 | 1.00 | 0.00 | 0.007 | 12.77 | 4.00 |
| ATOM | 2367 | HB1 | SER | 254 | 13.130 | −2.140 | 26.301 | 1.00 | 0.00 | 0.053 | 0.00 | 0.00 |
| ATOM | 2368 | HB2 | SER | 254 | 12.631 | −3.809 | 26.030 | 1.00 | 0.00 | 0.053 | 0.00 | 0.00 |
| ATOM | 2369 | OG | SER | 254 | 13.828 | −3.498 | 27.647 | 1.00 | 0.00 | −0.537 | 11.04 | −17.40 |
| ATOM | 2370 | HG | SER | 254 | 13.772 | −3.001 | 28.547 | 1.00 | 0.00 | 0.424 | 0.00 | 0.00 |
| ATOM | 2371 | C | SER | 254 | 10.903 | −4.125 | 28.021 | 1.00 | 0.00 | 0.396 | 9.82 | 4.00 |
| ATOM | 2372 | O | SER | 254 | 10.421 | −4.887 | 27.182 | 1.00 | 0.00 | −0.396 | 8.17 | −17.40 |
| ATOM | 2373 | N | SER | 255 | 10.930 | −4.405 | 29.323 | 1.00 | 0.00 | −0.650 | 9.00 | −17.40 |
| ATOM | 2374 | HN | SER | 255 | 11.344 | −3.725 | 29.977 | 1.00 | 0.00 | 0.440 | 0.00 | 0.00 |
| ATOM | 2375 | CA | SER | 255 | 10.382 | −5.660 | 29.842 | 1.00 | 0.00 | 0.158 | 9.40 | 4.00 |
| ATOM | 2376 | HA | SER | 255 | 10.462 | −6.442 | 29.087 | 1.00 | 0.00 | 0.053 | 0.00 | 0.00 |
| ATOM | 2377 | CB | SER | 255 | 11.165 | −6.152 | 31.069 | 1.00 | 0.00 | 0.007 | 12.77 | 4.00 |
| ATOM | 2378 | HB1 | SER | 255 | 12.241 | −6.034 | 30.941 | 1.00 | 0.00 | 0.053 | 0.00 | 0.00 |
| ATOM | 2379 | HB2 | SER | 255 | 10.985 | −7.207 | 31.272 | 1.00 | 0.00 | 0.053 | 0.00 | 0.00 |
| ATOM | 2380 | OG | SER | 255 | 10.811 | −5.441 | 32.247 | 1.00 | 0.00 | −0.537 | 11.04 | −17.40 |
| ATOM | 2381 | HG | SER | 255 | 11.211 | −4.492 | 32.209 | 1.00 | 0.00 | 0.424 | 0.00 | 0.00 |
| ATOM | 2382 | C | SER | 255 | 8.912 | −5.476 | 30.209 | 1.00 | 0.00 | 0.396 | 9.82 | 4.00 |
| ATOM | 2383 | O | SER | 255 | 8.248 | −6.420 | 30.659 | 1.00 | 0.00 | −0.396 | 8.17 | −17.40 |
| ATOM | 2384 | N | LYS | 256S | 8.402 | −4.257 | 30.022 | 1.00 | 0.00 | −0.650 | 9.00 | −17.40 |
| ATOM | 2385 | HN | LYS | 256S | 9.013 | −3.501 | 29.680 | 1.00 | 0.00 | 0.440 | 0.00 | 0.00 |
| ATOM | 2386 | CA | LYS | 256S | 6.985 | −3.969 | 30.293 | 1.00 | 0.00 | 0.158 | 9.40 | 4.00 |
| ATOM | 2387 | HA | LYS | 256S | 6.446 | −4.891 | 30.510 | 1.00 | 0.00 | 0.053 | 0.00 | 0.00 |
| ATOM | 2388 | CB | LYS | 256S | 6.843 | −3.047 | 31.504 | 1.00 | 0.00 | −0.106 | 12.77 | 4.00 |
| ATOM | 2389 | HB1 | LYS | 256S | 5.804 | −2.720 | 31.562 | 1.00 | 0.00 | 0.053 | 0.00 | 0.00 |
| ATOM | 2390 | HB2 | LYS | 256S | 7.508 | −2.195 | 31.362 | 1.00 | 0.00 | 0.053 | 0.00 | 0.00 |
| ATOM | 2391 | CG | LYS | 256S | 7.202 | −3.696 | 32.864 | 1.00 | 0.00 | −0.106 | 12.77 | 4.00 |
| ATOM | 2392 | HG1 | LYS | 256S | 7.191 | −2.978 | 33.684 | 1.00 | 0.00 | 0.053 | 0.00 | 0.00 |
| ATOM | 2393 | HG2 | LYS | 256S | 8.195 | −4.144 | 32.862 | 1.00 | 0.00 | 0.053 | 0.00 | 0.00 |
| ATOM | 2394 | CD | LYS | 256S | 6.221 | −4.819 | 33.259 | 1.00 | 0.00 | −0.106 | 12.77 | 4.00 |
| ATOM | 2395 | HD1 | LYS | 256S | 6.527 | −5.311 | 34.182 | 1.00 | 0.00 | 0.053 | 0.00 | 0.00 |
| ATOM | 2396 | HD2 | LYS | 256S | 6.159 | −5.585 | 32.486 | 1.00 | 0.00 | 0.053 | 0.00 | 0.00 |
| ATOM | 2397 | CE | LYS | 256S | 4.799 | −4.274 | 33.480 | 1.00 | 0.00 | 0.099 | 12.77 | 4.00 |
| ATOM | 2398 | HE1 | LYS | 256S | 4.404 | −3.795 | 32.583 | 1.00 | 0.00 | 0.053 | 0.00 | 0.00 |
| ATOM | 2399 | HE2 | LYS | 256S | 4.768 | −3.531 | 34.277 | 1.00 | 0.00 | 0.053 | 0.00 | 0.00 |
| ATOM | 2400 | NZ | LYS | 256S | 3.842 | −5.356 | 33.855 | 1.00 | 0.00 | −0.045 | 13.25 | −39.20 |
| ATOM | 2401 | HZ1 | LYS | 256S | 2.904 | −4.952 | 33.994 | 1.00 | 0.00 | 0.280 | 0.00 | 0.00 |
| ATOM | 2402 | HZ2 | LYS | 256S | 3.805 | −6.058 | 33.102 | 1.00 | 0.00 | 0.280 | 0.00 | 0.00 |
| ATOM | 2403 | HZ3 | LYS | 256S | 4.155 | −5.804 | 34.727 | 1.00 | 0.00 | 0.280 | 0.00 | 0.00 |
| ATOM | 2404 | C | LYS | 256S | 6.337 | −3.299 | 29.078 | 1.00 | 0.00 | 0.396 | 9.82 | 4.00 |
| ATOM | 2405 | O | LYS | 256S | 5.740 | −2.239 | 29.201 | 1.00 | 0.00 | −0.396 | 8.17 | −17.40 |
| ATOM | 2406 | N | PRO | 257 | 6.415 | −3.920 | 27.889 | 1.00 | 0.00 | −0.422 | 9.00 | −17.40 |
| ATOM | 2407 | CD | PRO | 257 | 6.890 | −5.261 | 27.482 | 1.00 | 0.00 | 0.105 | 12.77 | 4.00 |
| ATOM | 2408 | HD1 | PRO | 257 | 6.549 | −6.027 | 28.178 | 1.00 | 0.00 | 0.053 | 0.00 | 0.00 |
| ATOM | 2409 | HD2 | PRO | 257 | 7.978 | −5.300 | 27.451 | 1.00 | 0.00 | 0.053 | 0.00 | 0.00 |
| ATOM | 2410 | CA | PRO | 257 | 5.780 | −3.209 | 26.777 | 1.00 | 0.00 | 0.158 | 9.40 | 4.00 |
| ATOM | 2411 | HA | PRO | 257 | 6.170 | −2.191 | 26.753 | 1.00 | 0.00 | 0.053 | 0.00 | 0.00 |
| ATOM | 2412 | CB | PRO | 257 | 6.259 | −3.987 | 25.560 | 1.00 | 0.00 | −0.106 | 12.77 | 4.00 |
| ATOM | 2413 | HB1 | PRO | 257 | 7.247 | −3.656 | 25.240 | 1.00 | 0.00 | 0.053 | 0.00 | 0.00 |
| ATOM | 2414 | HB2 | PRO | 257 | 5.581 | −3.859 | 24.715 | 1.00 | 0.00 | 0.053 | 0.00 | 0.00 |
| ATOM | 2415 | CG | PRO | 257 | 6.269 | −5.434 | 26.089 | 1.00 | 0.00 | −0.106 | 12.77 | 4.00 |
| ATOM | 2416 | HG1 | PRO | 257 | 6.863 | −6.086 | 25.449 | 1.00 | 0.00 | 0.053 | 0.00 | 0.00 |
| ATOM | 2417 | HG2 | PRO | 257 | 5.261 | −5.847 | 26.133 | 1.00 | 0.00 | 0.053 | 0.00 | 0.00 |
| ATOM | 2418 | C | PRO | 257 | 4.254 | −3.119 | 26.876 | 1.00 | 0.00 | 0.396 | 9.82 | 4.00 |
| ATOM | 2419 | O | PRO | 257 | 3.606 | −2.371 | 26.114 | 1.00 | 0.00 | −0.396 | 8.17 | −17.40 |
| ATOM | 2420 | N | GLU | 258 | 3.675 | −3.848 | 27.829 | 1.00 | 0.00 | −0.650 | 9.00 | −17.40 |
| ATOM | 2421 | HN | GLU | 258 | 4.259 | −4.414 | 28.460 | 1.00 | 0.00 | 0.440 | 0.00 | 0.00 |
| ATOM | 2422 | CA | GLU | 258 | 2.226 | −3.856 | 27.992 | 1.00 | 0.00 | 0.158 | 9.40 | 4.00 |
| ATOM | 2423 | HA | GLU | 258 | 1.704 | −3.685 | 27.050 | 1.00 | 0.00 | 0.053 | 0.00 | 0.00 |
| ATOM | 2424 | CB | GLU | 258 | 1.753 | −5.235 | 28.507 | 1.00 | 0.00 | −0.106 | 12.77 | 4.00 |
| ATOM | 2425 | HB1 | GLU | 258 | 2.284 | −6.005 | 27.948 | 1.00 | 0.00 | 0.053 | 0.00 | 0.00 |
| ATOM | 2426 | HB2 | GLU | 258 | 0.678 | −5.308 | 28.340 | 1.00 | 0.00 | 0.053 | 0.00 | 0.00 |
| ATOM | 2427 | CG | GLU | 258 | 2.006 | −5.497 | 30.010 | 1.00 | 0.00 | −0.106 | 12.77 | 4.00 |
| ATOM | 2428 | HG1 | GLU | 258 | 1.236 | −6.178 | 30.372 | 1.00 | 0.00 | 0.053 | 0.00 | 0.00 |
| ATOM | 2429 | HG2 | GLU | 258 | 1.954 | −4.544 | 30.536 | 1.00 | 0.00 | 0.053 | 0.00 | 0.00 |
| ATOM | 2430 | CD | GLU | 258 | 3.374 | −6.129 | 30.323 | 1.00 | 0.00 | 0.399 | 9.82 | 4.00 |
| ATOM | 2431 | OE1 | GLU | 258 | 4.344 | −5.931 | 29.558 | 1.00 | 0.00 | −0.396 | 8.17 | −18.95 |
| ATOM | 2432 | OE2 | GLU | 258 | 3.485 | −6.813 | 31.360 | 1.00 | 0.00 | −0.427 | 8.17 | −18.95 |
| ATOM | 2433 | HE2 | GLU | 258 | 4.454 | −7.150 | 31.447 | 1.00 | 0.00 | 0.424 | 0.00 | 0.00 |
| ATOM | 2434 | C | GLU | 258 | 1.748 | −2.773 | 28.959 | 1.00 | 0.00 | 0.396 | 9.82 | 4.00 |
| ATOM | 2435 | O | GLU | 258 | 0.563 | −2.454 | 29.007 | 1.00 | 0.00 | −0.396 | 8.17 | −17.40 |
| ATOM | 2436 | N | LEU | 259 | 2.673 | −2.199 | 29.717 | 1.00 | 0.00 | −0.650 | 9.00 | −17.40 |
| ATOM | 2437 | HN | LEU | 259 | 3.661 | −2.461 | 29.599 | 1.00 | 0.00 | 0.440 | 0.00 | 0.00 |
| ATOM | 2438 | CA | LEU | 259 | 2.306 | −1.196 | 30.720 | 1.00 | 0.00 | 0.158 | 9.40 | 4.00 |
| ATOM | 2439 | HA | LEU | 259 | 1.564 | −1.637 | 31.386 | 1.00 | 0.00 | 0.053 | 0.00 | 0.00 |
| ATOM | 2440 | CB | LEU | 259 | 3.517 | −0.860 | 31.581 | 1.00 | 0.00 | −0.106 | 12.77 | 4.00 |
| ATOM | 2441 | HB1 | LEU | 259 | 4.256 | −0.364 | 30.952 | 1.00 | 0.00 | 0.053 | 0.00 | 0.00 |
| ATOM | 2442 | HB2 | LEU | 259 | 3.917 | −1.789 | 31.985 | 1.00 | 0.00 | 0.053 | 0.00 | 0.00 |
| ATOM | 2443 | CG | LEU | 259 | 3.215 | 0.066 | 32.759 | 1.00 | 0.00 | −0.053 | 9.40 | 4.00 |
| ATOM | 2444 | HG | LEU | 259 | 2.875 | 1.035 | 32.395 | 1.00 | 0.00 | 0.053 | 0.00 | 0.00 |
| ATOM | 2445 | CD1 | LEU | 259 | 2.127 | −0.539 | 33.634 | 1.00 | 0.00 | −0.159 | 16.15 | 4.00 |
| ATOM | 2446 | HD1 | LEU | 259 | 1.919 | 0.128 | 34.470 | 1.00 | 0.00 | 0.053 | 0.00 | 0.00 |
| ATOM | 2447 | HD1 | LEU | 259 | 1.220 | −0.675 | 33.045 | 1.00 | 0.00 | 0.053 | 0.00 | 0.00 |
| ATOM | 2448 | HD1 | LEU | 259 | 2.461 | −1.504 | 34.014 | 1.00 | 0.00 | 0.053 | 0.00 | 0.00 |
| ATOM | 2449 | CD2 | LEU | 259 | 4.491 | 0.289 | 33.530 | 1.00 | 0.00 | −0.159 | 16.15 | 4.00 |
| ATOM | 2450 | HD2 | LEU | 259 | 4.294 | 0.948 | 34.375 | 1.00 | 0.00 | 0.053 | 0.00 | 0.00 |
| ATOM | 2451 | HD2 | LEU | 259 | 4.867 | −0.666 | 33.895 | 1.00 | 0.00 | 0.053 | 0.00 | 0.00 |
| ATOM | 2452 | HD2 | LEU | 259 | 5.234 | 0.746 | 32.878 | 1.00 | 0.00 | 0.053 | 0.00 | 0.00 |
| ATOM | 2453 | C | LEU | 259 | 1.706 | 0.088 | 30.157 | 1.00 | 0.00 | 0.396 | 9.82 | 4.00 |
| ATOM | 2454 | O | LEU | 259 | 2.291 | 0.725 | 29.282 | 1.00 | 0.00 | −0.396 | 8.17 | −17.40 |
| ATOM | 2455 | N | LEU | 260 | 0.528 | 0.463 | 30.652 | 1.00 | 0.00 | −0.650 | 9.00 | −17.40 |
| ATOM | 2456 | HN | LEU | 260 | 0.071 | −0.123 | 31.365 | 1.00 | 0.00 | 0.440 | 0.00 | 0.00 |
| ATOM | 2457 | CA | LEU | 260 | −0.132 | 1.690 | 30.204 | 1.00 | 0.00 | 0.158 | 9.40 | 4.00 |
| ATOM | 2458 | HA | LEU | 260 | 0.476 | 2.185 | 29.447 | 1.00 | 0.00 | 0.053 | 0.00 | 0.00 |
| ATOM | 2459 | CB | LEU | 260 | −1.506 | 1.394 | 29.575 | 1.00 | 0.00 | −0.106 | 12.77 | 4.00 |
| ATOM | 2460 | HB1 | LEU | 260 | −1.901 | 2.326 | 29.171 | 1.00 | 0.00 | 0.053 | 0.00 | 0.00 |
| ATOM | 2461 | HB2 | LEU | 260 | −2.158 | 1.002 | 30.355 | 1.00 | 0.00 | 0.053 | 0.00 | 0.00 |
| ATOM | 2462 | CG | LEU | 260 | −1.492 | 0.380 | 28.444 | 1.00 | 0.00 | −0.053 | 9.40 | 4.00 |
| ATOM | 2463 | HG | LEU | 260 | −1.066 | −0.558 | 28.800 | 1.00 | 0.00 | 0.053 | 0.00 | 0.00 |
| ATOM | 2464 | CD1 | LEU | 260 | −2.931 | 0.135 | 27.954 | 1.00 | 0.00 | −0.159 | 16.15 | 4.00 |
| ATOM | 2465 | HD1 | LEU | 260 | −2.920 | −0.592 | 27.142 | 1.00 | 0.00 | 0.053 | 0.00 | 0.00 |
| ATOM | 2466 | HD1 | LEU | 260 | −3.535 | −0.247 | 28.776 | 1.00 | 0.00 | 0.053 | 0.00 | 0.00 |
| ATOM | 2467 | HD1 | LEU | 260 | −3.357 | 1.071 | 27.595 | 1.00 | 0.00 | 0.053 | 0.00 | 0.00 |
| ATOM | 2468 | CD2 | LEU | 260 | −0.608 | 0.909 | 27.308 | 1.00 | 0.00 | −0.159 | 16.15 | 4.00 |
| ATOM | 2469 | HD2 | LEU | 260 | −0.592 | 0.186 | 26.492 | 1.00 | 0.00 | 0.053 | 0.00 | 0.00 |
| ATOM | 2470 | HD2 | LEU | 260 | −1.008 | 1.856 | 26.946 | 1.00 | 0.00 | 0.053 | 0.00 | 0.00 |
| ATOM | 2471 | HD2 | LEU | 260 | 0.406 | 1.060 | 27.677 | 1.00 | 0.00 | 0.053 | 0.00 | 0.00 |
| ATOM | 2472 | C | LEU | 260 | −0.325 | 2.638 | 31.383 | 1.00 | 0.00 | 0.396 | 9.82 | 4.00 |
| ATOM | 2473 | O | LEU | 260 | −0.217 | 2.222 | 32.547 | 1.00 | 0.00 | −0.396 | 8.17 | −17.40 |
| ATOM | 2474 | N | TYR | 261 | −0.619 | 3.904 | 31.061 | 1.00 | 0.00 | −0.650 | 9.00 | −17.40 |
| ATOM | 2475 | HN | TYR | 261 | −0.710 | 4.142 | 30.063 | 1.00 | 0.00 | 0.440 | 0.00 | 0.00 |
| ATOM | 2476 | CA | TYR | 261 | −0.817 | 4.967 | 32.044 | 1.00 | 0.00 | 0.158 | 9.40 | 4.00 |
| ATOM | 2477 | HA | TYR | 261 | −1.092 | 4.583 | 33.026 | 1.00 | 0.00 | 0.053 | 0.00 | 0.00 |
| ATOM | 2478 | CB | TYR | 261 | 0.445 | 5.833 | 32.161 | 1.00 | 0.00 | −0.106 | 12.77 | 4.00 |
| ATOM | 2479 | HB1 | TYR | 261 | 0.248 | 6.597 | 32.913 | 1.00 | 0.00 | 0.053 | 0.00 | 0.00 |
| ATOM | 2480 | HB2 | TYR | 261 | 0.629 | 6.276 | 31.182 | 1.00 | 0.00 | 0.053 | 0.00 | 0.00 |
| ATOM | 2481 | CG | TYR | 261 | 1.735 | 5.144 | 32.573 | 1.00 | 0.00 | 0.000 | 7.26 | 0.60 |
| ATOM | 2482 | CD1 | TYR | 261 | 2.511 | 4.445 | 31.654 | 1.00 | 0.00 | −0.127 | 10.80 | 0.60 |
| ATOM | 2483 | HD1 | TYR | 261 | 2.165 | 4.341 | 30.625 | 1.00 | 0.00 | 0.127 | 0.00 | 0.00 |
| ATOM | 2484 | CE1 | TYR | 261 | 3.737 | 3.870 | 32.037 | 1.00 | 0.00 | −0.127 | 10.80 | 0.60 |
| ATOM | 2485 | HE1 | TYR | 261 | 4.344 | 3.328 | 31.311 | 1.00 | 0.00 | 0.127 | 0.00 | 0.00 |
| ATOM | 2486 | CD2 | TYR | 261 | 2.202 | 5.250 | 33.882 | 1.00 | 0.00 | −0.127 | 10.80 | 0.60 |
| ATOM | 2487 | HD2 | TYR | 261 | 1.604 | 5.789 | 34.617 | 1.00 | 0.00 | 0.127 | 0.00 | 0.00 |
| ATOM | 2488 | CE2 | TYR | 261 | 3.397 | 4.691 | 34.270 | 1.00 | 0.00 | −0.127 | 10.80 | 0.60 |
| ATOM | 2489 | HE2 | TYR | 261 | 3.738 | 4.792 | 35.300 | 1.00 | 0.00 | 0.127 | 0.00 | 0.00 |
| ATOM | 2490 | CZ | TYR | 261 | 4.162 | 4.002 | 33.352 | 1.00 | 0.00 | 0.026 | 7.26 | 0.60 |
| ATOM | 2491 | OH | TYR | 261 | 5.337 | 3.425 | 33.776 | 1.00 | 0.00 | −0.451 | 10.94 | −17.40 |
| ATOM | 2492 | HH | TYR | 261 | 5.223 | 3.082 | 34.741 | 1.00 | 0.00 | 0.424 | 0.00 | 0.00 |
| ATOM | 2493 | C | TYR | 261 | −1.925 | 5.941 | 31.641 | 1.00 | 0.00 | 0.396 | 9.82 | 4.00 |
| ATOM | 2494 | O | TYR | 261 | −2.400 | 5.940 | 30.503 | 1.00 | 0.00 | −0.396 | 8.17 | −17.40 |
| ATOM | 2495 | N | ARG | 262G | −2.298 | 6.799 | 32.590 | 1.00 | 0.00 | −0.650 | 9.00 | −17.40 |
| ATOM | 2496 | HN | ARG | 262G | −1.904 | 6.692 | 33.535 | 1.00 | 0.00 | 0.440 | 0.00 | 0.00 |
| ATOM | 2497 | CA | ARG | 262G | −3.240 | 7.882 | 32.343 | 1.00 | 0.00 | 0.158 | 9.40 | 4.00 |
| ATOM | 2498 | HA | ARG | 262G | −3.779 | 7.731 | 31.408 | 1.00 | 0.00 | 0.053 | 0.00 | 0.00 |
| ATOM | 2499 | CB | ARG | 262G | −4.244 | 8.052 | 33.510 | 1.00 | 0.00 | −0.106 | 12.77 | 4.00 |
| ATOM | 2500 | HB1 | ARG | 262G | −4.715 | 9.029 | 33.409 | 1.00 | 0.00 | 0.053 | 0.00 | 0.00 |
| ATOM | 2501 | HB2 | ARG | 262G | −3.688 | 7.982 | 34.445 | 1.00 | 0.00 | 0.053 | 0.00 | 0.00 |
| ATOM | 2502 | CG | ARG | 262G | −5.377 | 7.001 | 33.571 | 1.00 | 0.00 | −0.106 | 12.77 | 4.00 |
| ATOM | 2503 | HG1 | ARG | 262G | −4.977 | 5.993 | 33.688 | 1.00 | 0.00 | 0.053 | 0.00 | 0.00 |
| ATOM | 2504 | HG2 | ARG | 262G | −5.977 | 7.012 | 32.661 | 1.00 | 0.00 | 0.053 | 0.00 | 0.00 |
| ATOM | 2505 | CD | ARG | 262G | −6.332 | 7.268 | 34.766 | 1.00 | 0.00 | 0.374 | 12.77 | 4.00 |
| ATOM | 2506 | HD1 | ARG | 262G | −5.802 | 7.478 | 35.695 | 1.00 | 0.00 | 0.053 | 0.00 | 0.00 |
| ATOM | 2507 | HD2 | ARG | 262G | −6.987 | 6.424 | 34.984 | 1.00 | 0.00 | 0.053 | 0.00 | 0.00 |
| ATOM | 2508 | NE | ARG | 262G | −7.220 | 8.415 | 34.545 | 1.00 | 0.00 | −0.819 | 9.00 | −24.67 |
| ATOM | 2509 | HE | ARG | 262G | −6.838 | 9.357 | 34.707 | 1.00 | 0.00 | 0.407 | 0.00 | 0.00 |
| ATOM | 2510 | CZ | ARG | 262G | −8.491 | 8.316 | 34.147 | 1.00 | 0.00 | 0.796 | 6.95 | 4.00 |
| ATOM | 2511 | NH1 | ARG | 262G | −9.038 | 7.124 | 33.922 | 1.00 | 0.00 | −0.746 | 9.00 | −24.67 |
| ATOM | 2512 | HH1 | ARG | 262G | −10.019 | 7.057 | 33.615 | 1.00 | 0.00 | 0.407 | 0.00 | 0.00 |
| ATOM | 2513 | HH1 | ARG | 262G | −8.480 | 6.268 | 34.054 | 1.00 | 0.00 | 0.407 | 0.00 | 0.00 |
| ATOM | 2514 | NH2 | ARG | 262G | −9.224 | 9.411 | 33.971 | 1.00 | 0.00 | −0.746 | 9.00 | −24.67 |
| ATOM | 2515 | HH2 | ARG | 262G | −10.203 | 9.330 | 33.664 | 1.00 | 0.00 | 0.407 | 0.00 | 0.00 |
| ATOM | 2516 | HH2 | ARG | 262G | −8.812 | 10.339 | 34.141 | 1.00 | 0.00 | 0.407 | 0.00 | 0.00 |
| ATOM | 2517 | C | ARG | 262G | −2.318 | 9.119 | 32.290 | 1.00 | 0.00 | 0.396 | 9.82 | 4.00 |
| ATOM | 2518 | O | ARG | 262G | −1.275 | 9.140 | 32.968 | 1.00 | 0.00 | −0.396 | 8.17 | −17.40 |
| ATOM | 2519 | N | THR | 263 | −2.665 | 10.114 | 31.466 | 1.00 | 0.00 | −0.650 | 9.00 | −17.40 |
| ATOM | 2520 | HN | THR | 263 | −3.487 | 9.990 | 30.858 | 1.00 | 0.00 | 0.440 | 0.00 | 0.00 |
| ATOM | 2521 | CA | THR | 263 | −1.912 | 11.385 | 31.395 | 1.00 | 0.00 | 0.158 | 9.40 | 4.00 |
| ATOM | 2522 | HA | THR | 263 | −1.366 | 11.577 | 32.319 | 1.00 | 0.00 | 0.053 | 0.00 | 0.00 |
| ATOM | 2523 | CB | THR | 263 | −0.856 | 11.436 | 30.258 | 1.00 | 0.00 | 0.060 | 9.40 | 4.00 |
| ATOM | 2524 | HB | THR | 263 | −0.091 | 10.671 | 30.396 | 1.00 | 0.00 | 0.053 | 0.00 | 0.00 |
| ATOM | 2525 | OG1 | THR | 263 | −0.248 | 12.740 | 30.256 | 1.00 | 0.00 | −0.537 | 11.04 | −17.40 |
| ATOM | 2526 | HG1 | THR | 263 | 0.777 | 12.641 | 30.257 | 1.00 | 0.00 | 0.424 | 0.00 | 0.00 |
| ATOM | 2527 | CG2 | THR | 263 | −1.497 | 11.208 | 28.906 | 1.00 | 0.00 | −0.159 | 16.15 | 4.00 |
| ATOM | 2528 | HG2 | THR | 263 | −0.733 | 11.249 | 28.129 | 1.00 | 0.00 | 0.053 | 0.00 | 0.00 |
| ATOM | 2529 | HG2 | THR | 263 | −1.976 | 10.229 | 28.891 | 1.00 | 0.00 | 0.053 | 0.00 | 0.00 |
| ATOM | 2530 | HG2 | THR | 263 | −2.243 | 11.980 | 28.722 | 1.00 | 0.00 | 0.053 | 0.00 | 0.00 |
| ATOM | 2531 | C | THR | 263 | −2.845 | 12.576 | 31.157 | 1.00 | 0.00 | 0.396 | 9.82 | 4.00 |
| ATOM | 2532 | O | THR | 263 | −3.781 | 12.498 | 30.348 | 1.00 | 0.00 | −0.396 | 8.17 | −17.40 |
| ATOM | 2533 | N | ASP | 264P | −2.577 | 13.678 | 31.861 | 1.00 | 0.00 | −0.650 | 9.00 | −17.40 |
| ATOM | 2534 | HN | ASP | 264P | −1.789 | 13.659 | 32.524 | 1.00 | 0.00 | 0.440 | 0.00 | 0.00 |
| ATOM | 2535 | CA | ASP | 264P | −3.355 | 14.914 | 31.731 | 1.00 | 0.00 | 0.158 | 9.40 | 4.00 |
| ATOM | 2536 | HA | ASP | 264P | −4.408 | 14.655 | 31.623 | 1.00 | 0.00 | 0.053 | 0.00 | 0.00 |
| ATOM | 2537 | CB | ASP | 264P | −3.162 | 15.796 | 32.978 | 1.00 | 0.00 | −0.336 | 12.77 | 4.00 |
| ATOM | 2538 | HB1 | ASP | 264P | −3.551 | 16.805 | 32.844 | 1.00 | 0.00 | 0.053 | 0.00 | 0.00 |
| ATOM | 2539 | HB2 | ASP | 264P | −2.113 | 15.912 | 33.253 | 1.00 | 0.00 | 0.053 | 0.00 | 0.00 |
| ATOM | 2540 | CG | ASP | 264P | −3.857 | 15.238 | 34.203 | 1.00 | 0.00 | 0.297 | 9.82 | 4.00 |
| ATOM | 2541 | OD1 | ASP | 264P | −3.262 | 15.286 | 35.307 | 1.00 | 0.00 | −0.534 | 8.17 | −18.95 |
| ATOM | 2542 | OD2 | ASP | 264P | −5.012 | 14.774 | 34.066 | 1.00 | 0.00 | −0.534 | 8.17 | −18.95 |
| ATOM | 2543 | C | ASP | 264P | −2.871 | 15.671 | 30.500 | 1.00 | 0.00 | 0.396 | 9.82 | 4.00 |
| ATOM | 2544 | O | ASP | 264P | −3.581 | 16.531 | 29.953 | 1.00 | 0.00 | −0.396 | 8.17 | −17.40 |
| ATOM | 2545 | N | PHE | 265 | −1.663 | 15.335 | 30.058 | 1.00 | 0.00 | −0.650 | 9.00 | −17.40 |
| ATOM | 2546 | HN | PHE | 265 | −1.142 | 14.598 | 30.555 | 1.00 | 0.00 | 0.440 | 0.00 | 0.00 |
| ATOM | 2547 | CA | PHE | 265 | −1.044 | 15.969 | 28.894 | 1.00 | 0.00 | 0.158 | 9.40 | 4.00 |
| ATOM | 2548 | HA | PHE | 265 | −1.354 | 17.009 | 28.797 | 1.00 | 0.00 | 0.053 | 0.00 | 0.00 |
| ATOM | 2549 | CB | PHE | 265 | 0.495 | 15.978 | 29.053 | 1.00 | 0.00 | −0.106 | 12.77 | 4.00 |
| ATOM | 2550 | HB1 | PHE | 265 | 0.863 | 14.966 | 28.880 | 1.00 | 0.00 | 0.053 | 0.00 | 0.00 |
| ATOM | 2551 | HB2 | PHE | 265 | 0.733 | 16.305 | 30.065 | 1.00 | 0.00 | 0.053 | 0.00 | 0.00 |
| ATOM | 2552 | CG | PHE | 265 | 1.214 | 16.907 | 28.082 | 1.00 | 0.00 | 0.000 | 7.26 | 0.60 |
| ATOM | 2553 | CD1 | PHE | 265 | 1.598 | 18.185 | 28.468 | 1.00 | 0.00 | −0.127 | 10.80 | 0.60 |
| ATOM | 2554 | HD1 | PHE | 265 | 1.393 | 18.521 | 29.484 | 1.00 | 0.00 | 0.127 | 0.00 | 0.00 |
| ATOM | 2555 | CD2 | PHE | 265 | 1.492 | 16.499 | 26.772 | 1.00 | 0.00 | −0.127 | 10.80 | 0.60 |
| ATOM | 2556 | HD2 | PHE | 265 | 1.202 | 15.499 | 26.448 | 1.00 | 0.00 | 0.127 | 0.00 | 0.00 |
| ATOM | 2557 | CE1 | PHE | 265 | 2.241 | 19.046 | 27.578 | 1.00 | 0.00 | −0.127 | 10.80 | 0.60 |
| ATOM | 2558 | HE1 | PHE | 265 | 2.533 | 20.046 | 27.898 | 1.00 | 0.00 | 0.127 | 0.00 | 0.00 |
| ATOM | 2559 | CE2 | PHE | 265 | 2.133 | 17.351 | 25.876 | 1.00 | 0.00 | −0.127 | 10.80 | 0.60 |
| ATOM | 2560 | HE2 | PHE | 265 | 2.338 | 17.015 | 24.859 | 1.00 | 0.00 | 0.127 | 0.00 | 0.00 |
| ATOM | 2561 | CZ | PHE | 265 | 2.509 | 18.620 | 26.272 | 1.00 | 0.00 | −0.127 | 10.80 | 0.60 |
| ATOM | 2562 | HZ | PHE | 265 | 3.011 | 19.286 | 25.571 | 1.00 | 0.00 | 0.127 | 0.00 | 0.00 |
| ATOM | 2563 | C | PHE | 265 | −1.431 | 15.244 | 27.588 | 1.00 | 0.00 | 0.396 | 9.82 | 4.00 |
| ATOM | 2564 | O | PHE | 265 | −1.010 | 14.090 | 27.334 | 1.00 | 0.00 | −0.396 | 8.17 | −17.40 |
| ATOM | 2565 | N | PHE | 266 | −2.237 | 15.927 | 26.766 | 1.00 | 0.00 | −0.650 | 9.00 | −17.40 |
| ATOM | 2566 | HN | PHE | 266 | −2.554 | 16.864 | 27.052 | 1.00 | 0.00 | 0.440 | 0.00 | 0.00 |
| ATOM | 2567 | CA | PHE | 266 | −2.688 | 15.396 | 25.473 | 1.00 | 0.00 | 0.158 | 9.40 | 4.00 |
| ATOM | 2568 | HA | PHE | 266 | −3.193 | 14.443 | 25.632 | 1.00 | 0.00 | 0.053 | 0.00 | 0.00 |
| ATOM | 2569 | CB | PHE | 266 | −3.664 | 16.378 | 24.813 | 1.00 | 0.00 | −0.106 | 12.77 | 4.00 |
| ATOM | 2570 | HB1 | PHE | 266 | −3.267 | 17.389 | 24.721 | 1.00 | 0.00 | 0.053 | 0.00 | 0.00 |
| ATOM | 2571 | HB2 | PHE | 266 | −4.602 | 16.488 | 25.355 | 1.00 | 0.00 | 0.053 | 0.00 | 0.00 |
| ATOM | 2572 | CG | PHE | 266 | −4.061 | 15.982 | 23.416 | 1.00 | 0.00 | 0.000 | 7.26 | 0.60 |
| ATOM | 2573 | CD1 | PHE | 266 | −4.788 | 14.812 | 23.195 | 1.00 | 0.00 | −0.127 | 10.80 | 0.60 |
| ATOM | 2574 | HD1 | PHE | 266 | −5.095 | 14.204 | 24.046 | 1.00 | 0.00 | 0.127 | 0.00 | 0.00 |
| ATOM | 2575 | CD2 | PHE | 266 | −3.677 | 16.746 | 22.324 | 1.00 | 0.00 | −0.127 | 10.80 | 0.60 |
| ATOM | 2576 | HD2 | PHE | 266 | −3.111 | 17.664 | 22.480 | 1.00 | 0.00 | 0.127 | 0.00 | 0.00 |
| ATOM | 2577 | CE1 | PHE | 266 | −5.128 | 14.406 | 21.907 | 1.00 | 0.00 | −0.127 | 10.80 | 0.60 |
| ATOM | 2578 | HE1 | PHE | 266 | −5.695 | 13.488 | 21.752 | 1.00 | 0.00 | 0.127 | 0.00 | 0.00 |
| ATOM | 2579 | CE2 | PHE | 266 | −4.009 | 16.349 | 21.024 | 1.00 | 0.00 | −0.127 | 10.80 | 0.60 |
| ATOM | 2580 | HE2 | PHE | 266 | −3.696 | 16.954 | 20.173 | 1.00 | 0.00 | 0.127 | 0.00 | 0.00 |
| ATOM | 2581 | CZ | PHE | 266 | −4.738 | 15.182 | 20.816 | 1.00 | 0.00 | −0.127 | 10.80 | 0.60 |
| ATOM | 2582 | HZ | PHE | 266 | −5.003 | 14.875 | 19.804 | 1.00 | 0.00 | 0.127 | 0.00 | 0.00 |
| ATOM | 2583 | C | PHE | 266 | −1.468 | 15.189 | 24.556 | 1.00 | 0.00 | 0.396 | 9.82 | 4.00 |
| ATOM | 2584 | O | PHE | 266 | −0.809 | 16.171 | 24.150 | 1.00 | 0.00 | −0.396 | 8.17 | −17.40 |
| ATOM | 2585 | N | PRO | 267 | −1.155 | 13.928 | 24.193 | 1.00 | 0.00 | −0.422 | 9.00 | −17.40 |
| ATOM | 2586 | CD | PRO | 267 | −1.655 | 12.666 | 24.769 | 1.00 | 0.00 | 0.105 | 12.77 | 4.00 |
| ATOM | 2587 | HD1 | PRO | 267 | −2.328 | 12.151 | 24.083 | 1.00 | 0.00 | 0.053 | 0.00 | 0.00 |
| ATOM | 2588 | HD2 | PRO | 267 | −2.204 | 12.838 | 25.694 | 1.00 | 0.00 | 0.053 | 0.00 | 0.00 |
| ATOM | 2589 | CA | PRO | 267 | 0.015 | 13.667 | 23.324 | 1.00 | 0.00 | 0.158 | 9.40 | 4.00 |
| ATOM | 2590 | HA | PRO | 267 | 0.776 | 14.408 | 23.567 | 1.00 | 0.00 | 0.053 | 0.00 | 0.00 |
| ATOM | 2591 | CB | PRO | 267 | 0.433 | 12.239 | 23.704 | 1.00 | 0.00 | −0.106 | 12.77 | 4.00 |
| ATOM | 2592 | HB1 | PRO | 267 | 1.509 | 12.298 | 23.866 | 1.00 | 0.00 | 0.053 | 0.00 | 0.00 |
| ATOM | 2593 | HB2 | PRO | 267 | 0.152 | 11.629 | 22.845 | 1.00 | 0.00 | 0.053 | 0.00 | 0.00 |
| ATOM | 2594 | CG | PRO | 267 | −0.376 | 11.918 | 24.989 | 1.00 | 0.00 | −0.106 | 12.77 | 4.00 |
| ATOM | 2595 | HG1 | PRO | 267 | 0.149 | 12.259 | 25.881 | 1.00 | 0.00 | 0.053 | 0.00 | 0.00 |
| ATOM | 2596 | HG2 | PRO | 267 | −0.542 | 10.845 | 25.092 | 1.00 | 0.00 | 0.053 | 0.00 | 0.00 |
| ATOM | 2597 | C | PRO | 267 | −0.202 | 13.744 | 21.812 | 1.00 | 0.00 | 0.396 | 9.82 | 4.00 |
| ATOM | 2598 | O | PRO | 267 | 0.730 | 14.023 | 21.069 | 1.00 | 0.00 | −0.396 | 8.17 | −17.40 |
| ATOM | 2599 | N | GLY | 268 | −1.421 | 13.473 | 21.363 | 1.00 | 0.00 | −0.650 | 9.00 | −17.40 |
| ATOM | 2600 | HN | GLY | 268 | −2.180 | 13.260 | 22.025 | 1.00 | 0.00 | 0.440 | 0.00 | 0.00 |
| ATOM | 2601 | CA | GLY | 268 | −1.680 | 13.478 | 19.933 | 1.00 | 0.00 | 0.105 | 9.40 | 4.00 |
| ATOM | 2602 | HA1 | GLY | 268 | −1.359 | 14.457 | 19.579 | 1.00 | 0.00 | 0.053 | 0.00 | 0.00 |
| ATOM | 2603 | HA2 | GLY | 268 | −2.753 | 13.317 | 19.827 | 1.00 | 0.00 | 0.053 | 0.00 | 0.00 |
| ATOM | 2604 | C | GLY | 268 | −0.852 | 12.341 | 19.362 | 1.00 | 0.00 | 0.396 | 9.82 | 4.00 |
| ATOM | 2605 | O | GLY | 268 | −0.775 | 11.251 | 19.957 | 1.00 | 0.00 | −0.396 | 8.17 | −17.40 |
| ATOM | 2606 | N | LEU | 269 | −0.229 | 12.594 | 18.215 | 1.00 | 0.00 | −0.650 | 9.00 | −17.40 |
| ATOM | 2607 | HN | LEU | 269 | −0.364 | 13.518 | 17.780 | 1.00 | 0.00 | 0.440 | 0.00 | 0.00 |
| ATOM | 2608 | CA | LEU | 269 | 0.639 | 11.631 | 17.536 | 1.00 | 0.00 | 0.158 | 9.40 | 4.00 |
| ATOM | 2609 | HA | LEU | 269 | 0.735 | 11.937 | 16.494 | 1.00 | 0.00 | 0.053 | 0.00 | 0.00 |
| ATOM | 2610 | CB | LEU | 269 | 2.055 | 11.736 | 18.134 | 1.00 | 0.00 | −0.106 | 12.77 | 4.00 |
| ATOM | 2611 | HB1 | LEU | 269 | 2.710 | 11.034 | 17.618 | 1.00 | 0.00 | 0.053 | 0.00 | 0.00 |
| ATOM | 2612 | HB2 | LEU | 269 | 2.010 | 11.492 | 19.195 | 1.00 | 0.00 | 0.053 | 0.00 | 0.00 |
| ATOM | 2613 | CG | LEU | 269 | 2.575 | 13.180 | 17.945 | 1.00 | 0.00 | −0.053 | 9.40 | 4.00 |
| ATOM | 2614 | HG | LEU | 269 | 1.839 | 13.915 | 18.270 | 1.00 | 0.00 | 0.053 | 0.00 | 0.00 |
| ATOM | 2615 | CD1 | LEU | 269 | 3.852 | 13.426 | 18.748 | 1.00 | 0.00 | −0.159 | 16.15 | 4.00 |
| ATOM | 2616 | HD1 | LEU | 269 | 4.188 | 14.450 | 18.591 | 1.00 | 0.00 | 0.053 | 0.00 | 0.00 |
| ATOM | 2617 | HD1 | LEU | 269 | 3.651 | 13.268 | 19.807 | 1.00 | 0.00 | 0.053 | 0.00 | 0.00 |
| ATOM | 2618 | HD1 | LEU | 269 | 4.627 | 12.734 | 18.418 | 1.00 | 0.00 | 0.053 | 0.00 | 0.00 |
| ATOM | 2619 | CD2 | LEU | 269 | 2.796 | 13.431 | 16.456 | 1.00 | 0.00 | −0.159 | 16.15 | 4.00 |
| ATOM | 2620 | HD2 | LEU | 269 | 3.163 | 14.446 | 16.308 | 1.00 | 0.00 | 0.053 | 0.00 | 0.00 |
| ATOM | 2621 | HD2 | LEU | 269 | 3.528 | 12.721 | 16.072 | 1.00 | 0.00 | 0.053 | 0.00 | 0.00 |
| ATOM | 2622 | HD2 | LEU | 269 | 1.854 | 13.304 | 15.922 | 1.00 | 0.00 | 0.053 | 0.00 | 0.00 |
| ATOM | 2623 | C | LEU | 269 | 0.132 | 10.181 | 17.544 | 1.00 | 0.00 | 0.396 | 9.82 | 4.00 |
| ATOM | 2624 | O | LEU | 269 | 0.760 | 9.291 | 18.104 | 1.00 | 0.00 | −0.396 | 8.17 | −17.40 |
| ATOM | 2625 | N | GLY | 270 | −0.980 | 9.948 | 16.850 | 1.00 | 0.00 | −0.650 | 9.00 | −17.40 |
| ATOM | 2626 | HN | GLY | 270 | −1.422 | 10.718 | 16.328 | 1.00 | 0.00 | 0.440 | 0.00 | 0.00 |
| ATOM | 2627 | CA | GLY | 270 | −1.579 | 8.628 | 16.816 | 1.00 | 0.00 | 0.105 | 9.40 | 4.00 |
| ATOM | 2628 | HA1 | GLY | 270 | −0.740 | 7.942 | 16.941 | 1.00 | 0.00 | 0.053 | 0.00 | 0.00 |
| ATOM | 2629 | HA2 | GLY | 270 | −2.055 | 8.568 | 15.837 | 1.00 | 0.00 | 0.053 | 0.00 | 0.00 |
| ATOM | 2630 | C | GLY | 270 | −2.557 | 8.622 | 17.978 | 1.00 | 0.00 | 0.396 | 9.82 | 4.00 |
| ATOM | 2631 | O | GLY | 270 | −2.222 | 8.170 | 19.077 | 1.00 | 0.00 | −0.396 | 8.17 | −17.40 |
| ATOM | 2632 | N | TRP | 271 | −3.765 | 9.131 | 17.753 | 1.00 | 0.00 | −0.650 | 9.00 | −17.40 |
| ATOM | 2633 | HN | TRP | 271 | −4.013 | 9.484 | 16.817 | 1.00 | 0.00 | 0.440 | 0.00 | 0.00 |
| ATOM | 2634 | CA | TRP | 271 | −4.739 | 9.186 | 18.843 | 1.00 | 0.00 | 0.158 | 9.40 | 4.00 |
| ATOM | 2635 | HA | TRP | 271 | −4.468 | 8.425 | 19.575 | 1.00 | 0.00 | 0.053 | 0.00 | 0.00 |
| ATOM | 2636 | CB | TRP | 271 | −4.672 | 10.528 | 19.575 | 1.00 | 0.00 | −0.106 | 12.77 | 4.00 |
| ATOM | 2637 | HB1 | TRP | 271 | −3.666 | 10.646 | 19.979 | 1.00 | 0.00 | 0.053 | 0.00 | 0.00 |
| ATOM | 2638 | HB2 | TRP | 271 | −5.410 | 10.516 | 20.377 | 1.00 | 0.00 | 0.053 | 0.00 | 0.00 |
| ATOM | 2639 | CG | TRP | 271 | −4.958 | 11.724 | 18.713 | 1.00 | 0.00 | 0.000 | 7.26 | 0.60 |
| ATOM | 2640 | CD2 | TRP | 271 | −6.109 | 12.576 | 18.778 | 1.00 | 0.00 | 0.000 | 6.80 | 0.60 |
| ATOM | 2641 | CE2 | TRP | 271 | −5.923 | 13.603 | 17.819 | 1.00 | 0.00 | −0.050 | 6.80 | 0.60 |
| ATOM | 2642 | CD1 | TRP | 271 | −4.144 | 12.253 | 17.737 | 1.00 | 0.00 | −0.177 | 10.80 | 0.60 |
| ATOM | 2643 | HD1 | TRP | 271 | −3.182 | 11.838 | 17.433 | 1.00 | 0.00 | 0.127 | 0.00 | 0.00 |
| ATOM | 2644 | NE1 | TRP | 271 | −4.719 | 13.378 | 17.203 | 1.00 | 0.00 | −0.292 | 9.00 | −17.40 |
| ATOM | 2645 | HE1 | TRP | 271 | −4.311 | 13.960 | 16.458 | 1.00 | 0.00 | 0.393 | 0.00 | 0.00 |
| ATOM | 2646 | CE3 | TRP | 271 | −7.278 | 12.577 | 19.556 | 1.00 | 0.00 | −0.127 | 10.80 | 0.60 |
| ATOM | 2647 | HE3 | TRP | 271 | −7.450 | 11.798 | 20.299 | 1.00 | 0.00 | 0.127 | 0.00 | 0.00 |
| ATOM | 2648 | CZ2 | TRP | 271 | −6.865 | 14.625 | 17.616 | 1.00 | 0.00 | −0.127 | 10.80 | 0.60 |
| ATOM | 2649 | HZ2 | TRP | 271 | −6.702 | 15.402 | 16.870 | 1.00 | 0.00 | 0.127 | 0.00 | 0.00 |
| ATOM | 2650 | CZ3 | TRP | 271 | −8.216 | 13.595 | 19.360 | 1.00 | 0.00 | −0.127 | 10.80 | 0.60 |
| ATOM | 2651 | HZ3 | TRP | 271 | −9.126 | 13.610 | 19.959 | 1.00 | 0.00 | 0.127 | 0.00 | 0.00 |
| ATOM | 2652 | CH2 | TRP | 271 | −7.997 | 14.609 | 18.390 | 1.00 | 0.00 | −0.127 | 10.80 | 0.60 |
| ATOM | 2653 | HH2 | TRP | 271 | −8.744 | 15.391 | 18.258 | 1.00 | 0.00 | 0.127 | 0.00 | 0.00 |
| ATOM | 2654 | C | TRP | 271 | −6.170 | 8.934 | 18.444 | 1.00 | 0.00 | 0.396 | 9.82 | 4.00 |
| ATOM | 2655 | O | TRP | 271 | −6.626 | 9.351 | 17.370 | 1.00 | 0.00 | −0.396 | 8.17 | −17.40 |
| ATOM | 2656 | N | LEU | 272 | −6.869 | 8.255 | 19.344 | 1.00 | 0.00 | −0.650 | 9.00 | −17.40 |
| ATOM | 2657 | HN | LEU | 272 | −6.392 | 7.966 | 20.210 | 1.00 | 0.00 | 0.440 | 0.00 | 0.00 |
| ATOM | 2658 | CA | LEU | 272 | −8.264 | 7.895 | 19.180 | 1.00 | 0.00 | 0.158 | 9.40 | 4.00 |
| ATOM | 2659 | HA | LEU | 272 | −8.574 | 7.890 | 18.135 | 1.00 | 0.00 | 0.053 | 0.00 | 0.00 |
| ATOM | 2660 | CB | LEU | 272 | −8.473 | 6.479 | 19.737 | 1.00 | 0.00 | −0.106 | 12.77 | 4.00 |
| ATOM | 2661 | HB1 | LEU | 272 | −8.012 | 6.451 | 20.724 | 1.00 | 0.00 | 0.053 | 0.00 | 0.00 |
| ATOM | 2662 | HB2 | LEU | 272 | −7.989 | 5.788 | 19.046 | 1.00 | 0.00 | 0.053 | 0.00 | 0.00 |
| ATOM | 2663 | CG | LEU | 272 | −9.900 | 5.956 | 19.924 | 1.00 | 0.00 | −0.053 | 9.40 | 4.00 |
| ATOM | 2664 | HG | LEU | 272 | −10.527 | 6.735 | 20.356 | 1.00 | 0.00 | 0.053 | 0.00 | 0.00 |
| ATOM | 2665 | CD1 | LEU | 272 | −10.481 | 5.541 | 18.579 | 1.00 | 0.00 | −0.159 | 16.15 | 4.00 |
| ATOM | 2666 | HD1 | LEU | 272 | −11.496 | 5.170 | 18.720 | 1.00 | 0.00 | 0.053 | 0.00 | 0.00 |
| ATOM | 2667 | HD1 | LEU | 272 | −10.498 | 6.400 | 17.909 | 1.00 | 0.00 | 0.053 | 0.00 | 0.00 |
| ATOM | 2668 | HD1 | LEU | 272 | −9.864 | 4.754 | 18.143 | 1.00 | 0.00 | 0.053 | 0.00 | 0.00 |
| ATOM | 2669 | CD2 | LEU | 272 | −9.875 | 4.774 | 20.891 | 1.00 | 0.00 | −0.159 | 16.15 | 4.00 |
| ATOM | 2670 | HD2 | LEU | 272 | −10.887 | 4.395 | 21.029 | 1.00 | 0.00 | 0.053 | 0.00 | 0.00 |
| ATOM | 2671 | HD2 | LEU | 272 | −9.245 | 3.983 | 20.483 | 1.00 | 0.00 | 0.053 | 0.00 | 0.00 |
| ATOM | 2672 | HD2 | LEU | 272 | −9.474 | 5.098 | 21.851 | 1.00 | 0.00 | 0.053 | 0.00 | 0.00 |
| ATOM | 2673 | C | LEU | 272 | −9.213 | 8.865 | 19.907 | 1.00 | 0.00 | 0.396 | 9.82 | 4.00 |
| ATOM | 2674 | O | LEU | 272 | −8.983 | 9.236 | 21.060 | 1.00 | 0.00 | −0.396 | 8.17 | −17.40 |
| ATOM | 2675 | N | LEU | 273 | −10.273 | 9.283 | 19.227 | 1.00 | 0.00 | −0.650 | 9.00 | −17.40 |
| ATOM | 2676 | HN | LEU | 273 | −10.396 | 9.004 | 18.243 | 1.00 | 0.00 | 0.440 | 0.00 | 0.00 |
| ATOM | 2677 | CA | LEU | 273 | −11.258 | 10.133 | 19.870 | 1.00 | 0.00 | 0.158 | 9.40 | 4.00 |
| ATOM | 2678 | HA | LEU | 273 | −11.076 | 10.086 | 20.944 | 1.00 | 0.00 | 0.053 | 0.00 | 0.00 |
| ATOM | 2679 | CB | LEU | 273 | −11.142 | 11.590 | 19.424 | 1.00 | 0.00 | −0.106 | 12.77 | 4.00 |
| ATOM | 2680 | HB1 | LEU | 273 | −10.079 | 11.834 | 19.413 | 1.00 | 0.00 | 0.053 | 0.00 | 0.00 |
| ATOM | 2681 | HB2 | LEU | 273 | −11.690 | 12.188 | 20.151 | 1.00 | 0.00 | 0.053 | 0.00 | 0.00 |
| ATOM | 2682 | CG | LEU | 273 | −11.676 | 12.015 | 18.055 | 1.00 | 0.00 | −0.053 | 9.40 | 4.00 |
| ATOM | 2683 | HG | LEU | 273 | −12.651 | 11.547 | 17.923 | 1.00 | 0.00 | 0.053 | 0.00 | 0.00 |
| ATOM | 2684 | CD1 | LEU | 273 | −11.786 | 13.541 | 18.057 | 1.00 | 0.00 | −0.159 | 16.15 | 4.00 |
| ATOM | 2685 | HD1 | LEU | 273 | −12.165 | 13.879 | 17.092 | 1.00 | 0.00 | 0.053 | 0.00 | 0.00 |
| ATOM | 2686 | HD1 | LEU | 273 | −12.469 | 13.855 | 18.846 | 1.00 | 0.00 | 0.053 | 0.00 | 0.00 |
| ATOM | 2687 | HD1 | LEU | 273 | −10.802 | 13.976 | 18.233 | 1.00 | 0.00 | 0.053 | 0.00 | 0.00 |
| ATOM | 2688 | CD2 | LEU | 273 | −10.759 | 11.550 | 16.920 | 1.00 | 0.00 | −0.159 | 16.15 | 4.00 |
| ATOM | 2689 | HD2 | LEU | 273 | −11.172 | 11.871 | 15.963 | 1.00 | 0.00 | 0.053 | 0.00 | 0.00 |
| ATOM | 2690 | HD2 | LEU | 273 | −9.768 | 11.984 | 17.051 | 1.00 | 0.00 | 0.053 | 0.00 | 0.00 |
| ATOM | 2691 | HD2 | LEU | 273 | −10.683 | 10.462 | 16.935 | 1.00 | 0.00 | 0.053 | 0.00 | 0.00 |
| ATOM | 2692 | C | LEU | 273 | −12.634 | 9.586 | 19.512 | 1.00 | 0.00 | 0.396 | 9.82 | 4.00 |
| ATOM | 2693 | O | LEU | 273 | −12.797 | 8.866 | 18.521 | 1.00 | 0.00 | −0.396 | 8.17 | −17.40 |
| ATOM | 2694 | N | LEU | 274 | −13.616 | 9.893 | 20.351 | 1.00 | 0.00 | −0.650 | 9.00 | −17.40 |
| ATOM | 2695 | HN | LEU | 274 | −13.411 | 10.445 | 21.195 | 1.00 | 0.00 | 0.440 | 0.00 | 0.00 |
| ATOM | 2696 | CA | LEU | 274 | −14.977 | 9.457 | 20.089 | 1.00 | 0.00 | 0.158 | 9.40 | 4.00 |
| ATOM | 2697 | HA | LEU | 274 | −14.959 | 8.553 | 19.479 | 1.00 | 0.00 | 0.053 | 0.00 | 0.00 |
| ATOM | 2698 | CB | LEU | 274 | −15.713 | 9.180 | 21.391 | 1.00 | 0.00 | −0.106 | 12.77 | 4.00 |
| ATOM | 2699 | HB1 | LEU | 274 | −16.749 | 8.938 | 21.155 | 1.00 | 0.00 | 0.053 | 0.00 | 0.00 |
| ATOM | 2700 | HB2 | LEU | 274 | −15.660 | 10.074 | 22.012 | 1.00 | 0.00 | 0.053 | 0.00 | 0.00 |
| ATOM | 2701 | CG | LEU | 274 | −15.136 | 8.019 | 22.196 | 1.00 | 0.00 | −0.053 | 9.40 | 4.00 |
| ATOM | 2702 | HG | LEU | 274 | −14.107 | 8.267 | 22.455 | 1.00 | 0.00 | 0.053 | 0.00 | 0.00 |
| ATOM | 2703 | CD1 | LEU | 274 | −15.991 | 7.845 | 23.456 | 1.00 | 0.00 | −0.159 | 16.15 | 4.00 |
| ATOM | 2704 | HD1 | LEU | 274 | −15.599 | 7.019 | 24.050 | 1.00 | 0.00 | 0.053 | 0.00 | 0.00 |
| ATOM | 2705 | HD1 | LEU | 274 | −15.962 | 8.761 | 24.044 | 1.00 | 0.00 | 0.053 | 0.00 | 0.00 |
| ATOM | 2706 | HD1 | LEU | 274 | −17.020 | 7.629 | 23.170 | 1.00 | 0.00 | 0.053 | 0.00 | 0.00 |
| ATOM | 2707 | CD2 | LEU | 274 | −15.108 | 6.750 | 21.375 | 1.00 | 0.00 | −0.159 | 16.15 | 4.00 |
| ATOM | 2708 | HD2 | LEU | 274 | −14.692 | 5.938 | 21.973 | 1.00 | 0.00 | 0.053 | 0.00 | 0.00 |
| ATOM | 2709 | HD2 | LEU | 274 | −16.121 | 6.492 | 21.069 | 1.00 | 0.00 | 0.053 | 0.00 | 0.00 |
| ATOM | 2710 | HD2 | LEU | 274 | −14.489 | 6.902 | 20.490 | 1.00 | 0.00 | 0.053 | 0.00 | 0.00 |
| ATOM | 2711 | C | LEU | 274 | −15.724 | 10.547 | 19.352 | 1.00 | 0.00 | 0.396 | 9.82 | 4.00 |
| ATOM | 2712 | O | LEU | 274 | −15.391 | 11.724 | 19.453 | 1.00 | 0.00 | −0.396 | 8.17 | −17.40 |
| ATOM | 2713 | N | ALA | 275 | −16.741 | 10.137 | 18.610 | 1.00 | 0.00 | −0.650 | 9.00 | −17.40 |
| ATOM | 2714 | HN | ALA | 275 | −16.950 | 9.130 | 18.554 | 1.00 | 0.00 | 0.440 | 0.00 | 0.00 |
| ATOM | 2715 | CA | ALA | 275 | −17.566 | 11.075 | 17.875 | 1.00 | 0.00 | 0.158 | 9.40 | 4.00 |
| ATOM | 2716 | HA | ALA | 275 | −16.899 | 11.565 | 17.165 | 1.00 | 0.00 | 0.053 | 0.00 | 0.00 |
| ATOM | 2717 | CB | ALA | 275 | −18.689 | 10.335 | 17.151 | 1.00 | 0.00 | −0.159 | 16.15 | 4.00 |
| ATOM | 2718 | HB1 | ALA | 275 | −19.301 | 11.050 | 16.602 | 1.00 | 0.00 | 0.053 | 0.00 | 0.00 |
| ATOM | 2719 | HB2 | ALA | 275 | −18.260 | 9.615 | 16.454 | 1.00 | 0.00 | 0.053 | 0.00 | 0.00 |
| ATOM | 2720 | HB3 | ALA | 275 | −19.307 | 9.810 | 17.879 | 1.00 | 0.00 | 0.053 | 0.00 | 0.00 |
| ATOM | 2721 | C | ALA | 275 | −18.131 | 12.033 | 18.913 | 1.00 | 0.00 | 0.396 | 9.82 | 4.00 |
| ATOM | 2722 | O | ALA | 275 | −18.343 | 13.190 | 18.611 | 1.00 | 0.00 | −0.396 | 8.17 | −17.40 |
| ATOM | 2723 | N | GLU | 276 | −18.340 | 11.553 | 20.145 | 1.00 | 0.00 | −0.650 | 9.00 | −17.40 |
| ATOM | 2724 | HN | GLU | 276 | −18.126 | 10.566 | 20.349 | 1.00 | 0.00 | 0.440 | 0.00 | 0.00 |
| ATOM | 2725 | CA | GLU | 276 | −18.868 | 12.413 | 21.207 | 1.00 | 0.00 | 0.158 | 9.40 | 4.00 |
| ATOM | 2726 | HA | GLU | 276 | −19.847 | 12.815 | 20.949 | 1.00 | 0.00 | 0.053 | 0.00 | 0.00 |
| ATOM | 2727 | CB | GLU | 276 | −19.062 | 11.630 | 22.515 | 1.00 | 0.00 | −0.106 | 12.77 | 4.00 |
| ATOM | 2728 | HB1 | GLU | 276 | −19.113 | 12.351 | 23.330 | 1.00 | 0.00 | 0.053 | 0.00 | 0.00 |
| ATOM | 2729 | HB2 | GLU | 276 | −18.207 | 10.963 | 22.633 | 1.00 | 0.00 | 0.053 | 0.00 | 0.00 |
| ATOM | 2730 | CG | GLU | 276 | −20.317 | 10.764 | 22.609 | 1.00 | 0.00 | −0.106 | 12.77 | 4.00 |
| ATOM | 2731 | HG1 | GLU | 276 | −21.203 | 11.224 | 22.173 | 1.00 | 0.00 | 0.053 | 0.00 | 0.00 |
| ATOM | 2732 | HG2 | GLU | 276 | −20.605 | 10.516 | 23.630 | 1.00 | 0.00 | 0.053 | 0.00 | 0.00 |
| ATOM | 2733 | CD | GLU | 276 | −20.191 | 9.418 | 21.903 | 1.00 | 0.00 | 0.399 | 9.82 | 4.00 |
| ATOM | 2734 | OE1 | GLU | 276 | −20.937 | 8.475 | 22.285 | 1.00 | 0.00 | −0.396 | 8.17 | −18.95 |
| ATOM | 2735 | OE2 | GLU | 276 | −19.356 | 9.290 | 20.975 | 1.00 | 0.00 | −0.427 | 8.17 | −18.95 |
| ATOM | 2736 | HE2 | GLU | 276 | −19.394 | 8.328 | 20.608 | 1.00 | 0.00 | 0.424 | 0.00 | 0.00 |
| ATOM | 2737 | C | GLU | 276 | −17.943 | 13.609 | 21.481 | 1.00 | 0.00 | 0.396 | 9.82 | 4.00 |
| ATOM | 2738 | O | GLU | 276 | −18.411 | 14.708 | 21.780 | 1.00 | 0.00 | −0.396 | 8.17 | −17.40 |
| ATOM | 2739 | N | LEU | 277 | −16.631 | 13.399 | 21.397 | 1.00 | 0.00 | −0.650 | 9.00 | −17.40 |
| ATOM | 2740 | HN | LEU | 277 | −16.277 | 12.459 | 21.164 | 1.00 | 0.00 | 0.440 | 0.00 | 0.00 |
| ATOM | 2741 | CA | LEU | 277 | −15.689 | 14.490 | 21.632 | 1.00 | 0.00 | 0.158 | 9.40 | 4.00 |
| ATOM | 2742 | HA | LEU | 277 | −15.941 | 15.040 | 22.538 | 1.00 | 0.00 | 0.053 | 0.00 | 0.00 |
| ATOM | 2743 | CB | LEU | 277 | −14.251 | 13.977 | 21.846 | 1.00 | 0.00 | −0.106 | 12.77 | 4.00 |
| ATOM | 2744 | HB1 | LEU | 277 | −13.901 | 13.357 | 21.020 | 1.00 | 0.00 | 0.053 | 0.00 | 0.00 |
| ATOM | 2745 | HB2 | LEU | 277 | −14.154 | 13.368 | 22.745 | 1.00 | 0.00 | 0.053 | 0.00 | 0.00 |
| ATOM | 2746 | CG | LEU | 277 | −13.231 | 15.122 | 21.988 | 1.00 | 0.00 | −0.053 | 9.40 | 4.00 |
| ATOM | 2747 | HG | LEU | 277 | −13.255 | 15.743 | 21.092 | 1.00 | 0.00 | 0.053 | 0.00 | 0.00 |
| ATOM | 2748 | CD1 | LEU | 277 | −13.589 | 15.979 | 23.219 | 1.00 | 0.00 | −0.159 | 16.15 | 4.00 |
| ATOM | 2749 | HD1 | LEU | 277 | −12.867 | 16.789 | 23.320 | 1.00 | 0.00 | 0.053 | 0.00 | 0.00 |
| ATOM | 2750 | HD1 | LEU | 277 | −14.588 | 16.396 | 23.093 | 1.00 | 0.00 | 0.053 | 0.00 | 0.00 |
| ATOM | 2751 | HD1 | LEU | 277 | −13.564 | 15.357 | 24.114 | 1.00 | 0.00 | 0.053 | 0.00 | 0.00 |
| ATOM | 2752 | CD2 | LEU | 277 | −11.807 | 14.564 | 22.116 | 1.00 | 0.00 | −0.159 | 16.15 | 4.00 |
| ATOM | 2753 | HD2 | LEU | 277 | −11.100 | 15.388 | 22.215 | 1.00 | 0.00 | 0.053 | 0.00 | 0.00 |
| ATOM | 2754 | HD2 | LEU | 277 | −11.744 | 13.924 | 22.996 | 1.00 | 0.00 | 0.053 | 0.00 | 0.00 |
| ATOM | 2755 | HD2 | LEU | 277 | −11.562 | 13.982 | 21.227 | 1.00 | 0.00 | 0.053 | 0.00 | 0.00 |
| ATOM | 2756 | C | LEU | 277 | −15.692 | 15.479 | 20.474 | 1.00 | 0.00 | 0.396 | 9.82 | 4.00 |
| ATOM | 2757 | O | LEU | 277 | −15.628 | 16.702 | 20.686 | 1.00 | 0.00 | −0.396 | 8.17 | −17.40 |
| ATOM | 2758 | N | TRP | 278 | −15.738 | 14.964 | 19.251 | 1.00 | 0.00 | −0.650 | 9.00 | −17.40 |
| ATOM | 2759 | HN | TRP | 278 | −15.755 | 13.942 | 19.123 | 1.00 | 0.00 | 0.440 | 0.00 | 0.00 |
| ATOM | 2760 | CA | TRP | 278 | −15.763 | 15.838 | 18.097 | 1.00 | 0.00 | 0.158 | 9.40 | 4.00 |
| ATOM | 2761 | HA | TRP | 278 | −14.854 | 16.439 | 18.074 | 1.00 | 0.00 | 0.053 | 0.00 | 0.00 |
| ATOM | 2762 | CB | TRP | 278 | −15.800 | 15.026 | 16.804 | 1.00 | 0.00 | −0.106 | 12.77 | 4.00 |
| ATOM | 2763 | HB1 | TRP | 278 | −16.703 | 14.422 | 16.717 | 1.00 | 0.00 | 0.053 | 0.00 | 0.00 |
| ATOM | 2764 | HB2 | TRP | 278 | −14.960 | 14.335 | 16.722 | 1.00 | 0.00 | 0.053 | 0.00 | 0.00 |
| ATOM | 2765 | CG | TRP | 278 | −15.753 | 15.892 | 15.584 | 1.00 | 0.00 | 0.000 | 7.26 | 0.60 |
| ATOM | 2766 | CD2 | TRP | 278 | −14.755 | 16.866 | 15.277 | 1.00 | 0.00 | 0.000 | 6.80 | 0.60 |
| ATOM | 2767 | CE2 | TRP | 278 | −15.145 | 17.502 | 14.076 | 1.00 | 0.00 | −0.050 | 6.80 | 0.60 |
| ATOM | 2768 | CD1 | TRP | 278 | −16.685 | 15.964 | 14.578 | 1.00 | 0.00 | −0.177 | 10.80 | 0.60 |
| ATOM | 2769 | HD1 | TRP | 278 | −17.578 | 15.343 | 14.508 | 1.00 | 0.00 | 0.127 | 0.00 | 0.00 |
| ATOM | 2770 | NE1 | TRP | 278 | −16.326 | 16.935 | 13.669 | 1.00 | 0.00 | −0.292 | 9.00 | −17.40 |
| ATOM | 2771 | HE1 | TRP | 278 | −16.856 | 17.192 | 12.824 | 1.00 | 0.00 | 0.393 | 0.00 | 0.00 |
| ATOM | 2772 | CE3 | TRP | 278 | −13.566 | 17.267 | 15.906 | 1.00 | 0.00 | −0.127 | 10.80 | 0.60 |
| ATOM | 2773 | HE3 | TRP | 278 | −13.240 | 16.792 | 16.832 | 1.00 | 0.00 | 0.127 | 0.00 | 0.00 |
| ATOM | 2774 | CZ2 | TRP | 278 | −14.386 | 18.522 | 13.491 | 1.00 | 0.00 | −0.127 | 10.80 | 0.60 |
| ATOM | 2775 | HZ2 | TRP | 278 | −14.700 | 19.002 | 12.564 | 1.00 | 0.00 | 0.127 | 0.00 | 0.00 |
| ATOM | 2776 | CZ3 | TRP | 278 | −12.813 | 18.278 | 15.327 | 1.00 | 0.00 | −0.127 | 10.80 | 0.60 |
| ATOM | 2777 | HZ3 | TRP | 278 | −11.887 | 18.599 | 15.805 | 1.00 | 0.00 | 0.127 | 0.00 | 0.00 |
| ATOM | 2778 | CH2 | TRP | 278 | −13.225 | 18.894 | 14.134 | 1.00 | 0.00 | −0.127 | 10.80 | 0.60 |
| ATOM | 2779 | HH2 | TRP | 278 | −12.611 | 19.686 | 13.705 | 1.00 | 0.00 | 0.127 | 0.00 | 0.00 |
| ATOM | 2780 | C | TRP | 278 | −16.979 | 16.760 | 18.179 | 1.00 | 0.00 | 0.396 | 9.82 | 4.00 |
| ATOM | 2781 | O | TRP | 278 | −16.921 | 17.912 | 17.751 | 1.00 | 0.00 | −0.396 | 8.17 | −17.40 |
| ATOM | 2782 | N | ALA | 279 | −18.086 | 16.252 | 18.725 | 1.00 | 0.00 | −0.650 | 9.00 | −17.40 |
| ATOM | 2783 | HN | ALA | 279 | −18.086 | 15.272 | 19.042 | 1.00 | 0.00 | 0.440 | 0.00 | 0.00 |
| ATOM | 2784 | CA | ALA | 279 | −19.302 | 17.058 | 18.883 | 1.00 | 0.00 | 0.158 | 9.40 | 4.00 |
| ATOM | 2785 | HA | ALA | 279 | −19.623 | 17.435 | 17.912 | 1.00 | 0.00 | 0.053 | 0.00 | 0.00 |
| ATOM | 2786 | CB | ALA | 279 | −20.441 | 16.201 | 19.461 | 1.00 | 0.00 | −0.159 | 16.15 | 4.00 |
| ATOM | 2787 | HB1 | ALA | 279 | −21.336 | 16.812 | 19.572 | 1.00 | 0.00 | 0.053 | 0.00 | 0.00 |
| ATOM | 2788 | HB2 | ALA | 279 | −20.650 | 15.371 | 18.786 | 1.00 | 0.00 | 0.053 | 0.00 | 0.00 |
| ATOM | 2789 | HB3 | ALA | 279 | −20.144 | 15.810 | 20.434 | 1.00 | 0.00 | 0.053 | 0.00 | 0.00 |
| ATOM | 2790 | C | ALA | 279 | −19.023 | 18.243 | 19.816 | 1.00 | 0.00 | 0.396 | 9.82 | 4.00 |
| ATOM | 2791 | O | ALA | 279 | −19.557 | 19.342 | 19.631 | 1.00 | 0.00 | −0.396 | 8.17 | −17.40 |
| ATOM | 2792 | N | GLU | 280 | −18.165 | 18.011 | 20.804 | 1.00 | 0.00 | −0.650 | 9.00 | −17.40 |
| ATOM | 2793 | HN | GLU | 280 | −17.744 | 17.074 | 20.885 | 1.00 | 0.00 | 0.440 | 0.00 | 0.00 |
| ATOM | 2794 | CA | GLU | 280 | −17.797 | 19.026 | 21.773 | 1.00 | 0.00 | 0.158 | 9.40 | 4.00 |
| ATOM | 2795 | HA | GLU | 280 | −18.653 | 19.594 | 22.135 | 1.00 | 0.00 | 0.053 | 0.00 | 0.00 |
| ATOM | 2796 | CB | GLU | 280 | −17.154 | 18.339 | 22.983 | 1.00 | 0.00 | −0.106 | 12.77 | 4.00 |
| ATOM | 2797 | HB1 | GLU | 280 | −16.114 | 18.119 | 22.738 | 1.00 | 0.00 | 0.053 | 0.00 | 0.00 |
| ATOM | 2798 | HB2 | GLU | 280 | −17.704 | 17.420 | 23.185 | 1.00 | 0.00 | 0.053 | 0.00 | 0.00 |
| ATOM | 2799 | CG | GLU | 280 | −17.160 | 19.154 | 24.236 | 1.00 | 0.00 | −0.106 | 12.77 | 4.00 |
| ATOM | 2800 | HG1 | GLU | 280 | −18.163 | 19.361 | 24.607 | 1.00 | 0.00 | 0.053 | 0.00 | 0.00 |
| ATOM | 2801 | HG2 | GLU | 280 | −16.685 | 20.128 | 24.115 | 1.00 | 0.00 | 0.053 | 0.00 | 0.00 |
| ATOM | 2802 | CD | GLU | 280 | −16.428 | 18.483 | 25.389 | 1.00 | 0.00 | 0.399 | 9.82 | 4.00 |
| ATOM | 2803 | OE1 | GLU | 280 | −16.567 | 17.254 | 25.575 | 1.00 | 0.00 | −0.396 | 8.17 | −18.95 |
| ATOM | 2804 | OE2 | GLU | 280 | −15.733 | 19.205 | 26.129 | 1.00 | 0.00 | −0.427 | 8.17 | −18.95 |
| ATOM | 2805 | HE2 | GLU | 280 | −15.319 | 18.632 | 26.878 | 1.00 | 0.00 | 0.424 | 0.00 | 0.00 |
| ATOM | 2806 | C | GLU | 280 | −16.808 | 20.066 | 21.205 | 1.00 | 0.00 | 0.396 | 9.82 | 4.00 |
| ATOM | 2807 | O | GLU | 280 | −16.924 | 21.268 | 21.462 | 1.00 | 0.00 | −0.396 | 8.17 | −17.40 |
| ATOM | 2808 | N | LEU | 281 | −15.648 | 19.596 | 20.418 | 1.00 | 0.00 | −0.650 | 9.00 | −17.40 |
| ATOM | 2809 | HN | LEU | 281 | −15.847 | 18.594 | 20.176 | 1.00 | 0.00 | 0.440 | 0.00 | 0.00 |
| ATOM | 2810 | CA | LEU | 281 | −14.795 | 20.451 | 19.884 | 1.00 | 0.00 | 0.158 | 9.40 | 4.00 |
| ATOM | 2811 | HA | LEU | 281 | −14.547 | 21.183 | 20.652 | 1.00 | 0.00 | 0.053 | 0.00 | 0.00 |
| ATOM | 2812 | CB | LEU | 281 | −13.532 | 19.606 | 19.655 | 1.00 | 0.00 | −0.106 | 12.77 | 4.00 |
| ATOM | 2813 | HB1 | LEU | 281 | −12.729 | 20.274 | 19.341 | 1.00 | 0.00 | 0.053 | 0.00 | 0.00 |
| ATOM | 2814 | HB2 | LEU | 281 | −13.751 | 18.872 | 18.879 | 1.00 | 0.00 | 0.053 | 0.00 | 0.00 |
| ATOM | 2815 | CG | LEU | 281 | −13.029 | 18.831 | 20.893 | 1.00 | 0.00 | −0.053 | 9.40 | 4.00 |
| ATOM | 2816 | HG | LEU | 281 | −13.787 | 18.113 | 21.205 | 1.00 | 0.00 | 0.053 | 0.00 | 0.00 |
| ATOM | 2817 | CD1 | LEU | 281 | −11.737 | 18.087 | 20.539 | 1.00 | 0.00 | −0.159 | 16.15 | 4.00 |
| ATOM | 2818 | HD1 | LEU | 281 | −11.381 | 17.539 | 21.412 | 1.00 | 0.00 | 0.053 | 0.00 | 0.00 |
| ATOM | 2819 | HD1 | LEU | 281 | −11.931 | 17.386 | 19.726 | 1.00 | 0.00 | 0.053 | 0.00 | 0.00 |
| ATOM | 2820 | HD1 | LEU | 281 | −10.978 | 18.804 | 20.226 | 1.00 | 0.00 | 0.053 | 0.00 | 0.00 |
| ATOM | 2821 | CD2 | LEU | 281 | −12.803 | 19.793 | 22.071 | 1.00 | 0.00 | −0.159 | 16.15 | 4.00 |
| ATOM | 2822 | HD2 | LEU | 281 | −12.448 | 19.232 | 22.936 | 1.00 | 0.00 | 0.053 | 0.00 | 0.00 |
| ATOM | 2823 | HD2 | LEU | 281 | −12.059 | 20.539 | 21.793 | 1.00 | 0.00 | 0.053 | 0.00 | 0.00 |
| ATOM | 2824 | HD2 | LEU | 281 | −13.740 | 20.289 | 22.320 | 1.00 | 0.00 | 0.053 | 0.00 | 0.00 |
| ATOM | 2825 | C | LEU | 281 | −15.100 | 21.234 | 18.625 | 1.00 | 0.00 | 0.396 | 9.82 | 4.00 |
| ATOM | 2826 | O | LEU | 281 | −14.793 | 22.427 | 18.534 | 1.00 | 0.00 | −0.396 | 8.17 | −17.40 |
| ATOM | 2827 | N | GLU | 282 | −15.707 | 20.573 | 17.651 | 1.00 | 0.00 | −0.650 | 9.00 | −17.40 |
| ATOM | 2828 | HN | GLU | 282 | −15.985 | 19.591 | 17.793 | 1.00 | 0.00 | 0.440 | 0.00 | 0.00 |
| ATOM | 2829 | CA | GLU | 282 | −15.983 | 21.230 | 16.374 | 1.00 | 0.00 | 0.158 | 9.40 | 4.00 |
| ATOM | 2830 | HA | GLU | 282 | −15.037 | 21.327 | 15.841 | 1.00 | 0.00 | 0.053 | 0.00 | 0.00 |
| ATOM | 2831 | CB | GLU | 282 | −16.847 | 20.326 | 15.480 | 1.00 | 0.00 | −0.106 | 12.77 | 4.00 |
| ATOM | 2832 | HB1 | GLU | 282 | −17.813 | 20.078 | 15.918 | 1.00 | 0.00 | 0.053 | 0.00 | 0.00 |
| ATOM | 2833 | HB2 | GLU | 282 | −16.381 | 19.366 | 15.254 | 1.00 | 0.00 | 0.053 | 0.00 | 0.00 |
| ATOM | 2834 | CG | GLU | 282 | −17.172 | 20.949 | 14.111 | 1.00 | 0.00 | −0.106 | 12.77 | 4.00 |
| ATOM | 2835 | HG1 | GLU | 282 | −16.287 | 21.337 | 13.606 | 1.00 | 0.00 | 0.053 | 0.00 | 0.00 |
| ATOM | 2836 | HG2 | GLU | 282 | −17.869 | 21.783 | 14.185 | 1.00 | 0.00 | 0.053 | 0.00 | 0.00 |
| ATOM | 2837 | CD | GLU | 282 | −17.802 | 19.956 | 13.153 | 1.00 | 0.00 | 0.399 | 9.82 | 4.00 |
| ATOM | 2838 | OE1 | GLU | 282 | −17.146 | 19.593 | 12.148 | 1.00 | 0.00 | −0.396 | 8.17 | −18.95 |
| ATOM | 2839 | OE2 | GLU | 282 | −18.945 | 19.518 | 13.403 | 1.00 | 0.00 | −0.427 | 8.17 | −18.95 |
| ATOM | 2840 | HE2 | GLU | 282 | −19.223 | 18.847 | 12.672 | 1.00 | 0.00 | 0.424 | 0.00 | 0.00 |
| ATOM | 2841 | C | GLU | 282 | −16.599 | 22.626 | 16.435 | 1.00 | 0.00 | 0.396 | 9.82 | 4.00 |
| ATOM | 2842 | O | GLU | 282 | −16.173 | 23.517 | 15.706 | 1.00 | 0.00 | −0.396 | 8.17 | −17.40 |
| ATOM | 2843 | N | PRO | 283 | −17.591 | 22.845 | 17.312 | 1.00 | 0.00 | −0.422 | 9.00 | −17.40 |
| ATOM | 2844 | CD | PRO | 283 | −18.223 | 21.902 | 18.254 | 1.00 | 0.00 | 0.105 | 12.77 | 4.00 |
| ATOM | 2845 | HD1 | PRO | 283 | −17.657 | 21.832 | 19.183 | 1.00 | 0.00 | 0.053 | 0.00 | 0.00 |
| ATOM | 2846 | HD2 | PRO | 283 | −18.283 | 20.900 | 17.829 | 1.00 | 0.00 | 0.053 | 0.00 | 0.00 |
| ATOM | 2847 | CA | PRO | 283 | −18.213 | 24.169 | 17.393 | 1.00 | 0.00 | 0.158 | 9.40 | 4.00 |
| ATOM | 2848 | HA | PRO | 283 | −18.650 | 24.402 | 16.422 | 1.00 | 0.00 | 0.053 | 0.00 | 0.00 |
| ATOM | 2849 | CB | PRO | 283 | −19.286 | 23.995 | 18.478 | 1.00 | 0.00 | −0.106 | 12.77 | 4.00 |
| ATOM | 2850 | HB1 | PRO | 283 | −20.173 | 24.583 | 18.245 | 1.00 | 0.00 | 0.053 | 0.00 | 0.00 |
| ATOM | 2851 | HB2 | PRO | 283 | −18.912 | 24.318 | 19.449 | 1.00 | 0.00 | 0.053 | 0.00 | 0.00 |
| ATOM | 2852 | CG | PRO | 283 | −19.582 | 22.513 | 18.462 | 1.00 | 0.00 | −0.106 | 12.77 | 4.00 |
| ATOM | 2853 | HG1 | PRO | 283 | −20.271 | 22.258 | 17.657 | 1.00 | 0.00 | 0.053 | 0.00 | 0.00 |
| ATOM | 2854 | HG2 | PRO | 283 | −20.034 | 22.192 | 19.400 | 1.00 | 0.00 | 0.053 | 0.00 | 0.00 |
| ATOM | 2855 | C | PRO | 283 | −17.242 | 25.292 | 17.748 | 1.00 | 0.00 | 0.396 | 9.82 | 4.00 |
| ATOM | 2856 | O | PRO | 283 | −17.450 | 26.445 | 17.368 | 1.00 | 0.00 | −0.396 | 8.17 | −17.40 |
| ATOM | 2857 | N | LYS | 284S | −16.192 | 24.960 | 18.490 | 1.00 | 0.00 | −0.650 | 9.00 | −17.40 |
| ATOM | 2858 | HN | LYS | 284S | −16.050 | 23.977 | 18.763 | 1.00 | 0.00 | 0.440 | 0.00 | 0.00 |
| ATOM | 2859 | CA | LYS | 284S | −15.242 | 25.971 | 18.920 | 1.00 | 0.00 | 0.158 | 9.40 | 4.00 |
| ATOM | 2860 | HA | LYS | 284S | −15.530 | 26.976 | 18.612 | 1.00 | 0.00 | 0.053 | 0.00 | 0.00 |
| ATOM | 2861 | CB | LYS | 284S | −15.192 | 26.017 | 20.461 | 1.00 | 0.00 | −0.106 | 12.77 | 4.00 |
| ATOM | 2862 | HB1 | LYS | 284S | −16.116 | 26.470 | 20.819 | 1.00 | 0.00 | 0.053 | 0.00 | 0.00 |
| ATOM | 2863 | HB2 | LYS | 284S | −14.330 | 26.615 | 20.757 | 1.00 | 0.00 | 0.053 | 0.00 | 0.00 |
| ATOM | 2864 | CG | LYS | 284S | −15.057 | 24.648 | 21.131 | 1.00 | 0.00 | −0.106 | 12.77 | 4.00 |
| ATOM | 2865 | HG1 | LYS | 284S | −14.229 | 24.067 | 20.724 | 1.00 | 0.00 | 0.053 | 0.00 | 0.00 |
| ATOM | 2866 | HG2 | LYS | 284S | −15.951 | 24.037 | 21.009 | 1.00 | 0.00 | 0.053 | 0.00 | 0.00 |
| ATOM | 2867 | CD | LYS | 284S | −14.807 | 24.760 | 22.652 | 1.00 | 0.00 | −0.106 | 12.77 | 4.00 |
| ATOM | 2868 | HD1 | LYS | 284S | −15.610 | 25.308 | 23.144 | 1.00 | 0.00 | 0.053 | 0.00 | 0.00 |
| ATOM | 2869 | HD2 | LYS | 284S | −13.873 | 25.282 | 22.860 | 1.00 | 0.00 | 0.053 | 0.00 | 0.00 |
| ATOM | 2870 | CE | LYS | 284S | −14.721 | 23.370 | 23.305 | 1.00 | 0.00 | 0.099 | 12.77 | 4.00 |
| ATOM | 2871 | HE1 | LYS | 284S | −14.056 | 22.707 | 22.750 | 1.00 | 0.00 | 0.053 | 0.00 | 0.00 |
| ATOM | 2872 | HE2 | LYS | 284S | −15.697 | 22.887 | 23.349 | 1.00 | 0.00 | 0.053 | 0.00 | 0.00 |
| ATOM | 2873 | NZ | LYS | 284S | −14.204 | 23.452 | 24.696 | 1.00 | 0.00 | −0.045 | 13.25 | −39.20 |
| ATOM | 2874 | HZ1 | LYS | 284S | −14.159 | 22.506 | 25.102 | 1.00 | 0.00 | 0.280 | 0.00 | 0.00 |
| ATOM | 2875 | HZ2 | LYS | 284S | −13.261 | 23.866 | 24.688 | 1.00 | 0.00 | 0.280 | 0.00 | 0.00 |
| ATOM | 2876 | HZ3 | LYS | 284S | −14.832 | 24.039 | 25.262 | 1.00 | 0.00 | 0.280 | 0.00 | 0.00 |
| ATOM | 2877 | C | LYS | 284S | −13.851 | 25.742 | 18.360 | 1.00 | 0.00 | 0.396 | 9.82 | 4.00 |
| ATOM | 2878 | O | LYS | 284S | −12.887 | 26.309 | 18.880 | 1.00 | 0.00 | −0.396 | 8.17 | −17.40 |
| ATOM | 2879 | N | TRP | 285 | −13.740 | 24.920 | 17.312 | 1.00 | 0.00 | −0.650 | 9.00 | −17.40 |
| ATOM | 2880 | HN | TRP | 285 | −14.587 | 24.482 | 16.922 | 1.00 | 0.00 | 0.440 | 0.00 | 0.00 |
| ATOM | 2881 | CA | TRP | 285 | −12.440 | 24.628 | 16.707 | 1.00 | 0.00 | 0.158 | 9.40 | 4.00 |
| ATOM | 2882 | HA | TRP | 285 | −11.825 | 24.024 | 17.374 | 1.00 | 0.00 | 0.053 | 0.00 | 0.00 |
| ATOM | 2883 | CB | TRP | 285 | −12.623 | 23.786 | 15.433 | 1.00 | 0.00 | −0.106 | 12.77 | 4.00 |
| ATOM | 2884 | HB1 | TRP | 285 | −12.964 | 24.442 | 14.632 | 1.00 | 0.00 | 0.053 | 0.00 | 0.00 |
| ATOM | 2885 | HB2 | TRP | 285 | −13.364 | 23.012 | 15.635 | 1.00 | 0.00 | 0.053 | 0.00 | 0.00 |
| ATOM | 2886 | CG | TRP | 285 | −11.363 | 23.106 | 14.966 | 1.00 | 0.00 | 0.000 | 7.26 | 0.60 |
| ATOM | 2887 | CD2 | TRP | 285 | −10.711 | 21.989 | 15.585 | 1.00 | 0.00 | 0.000 | 6.80 | 0.60 |
| ATOM | 2888 | CE2 | TRP | 285 | −9.589 | 21.667 | 14.791 | 1.00 | 0.00 | −0.050 | 6.80 | 7.60 |
| ATOM | 2889 | CD1 | TRP | 285 | −10.621 | 23.414 | 13.847 | 1.00 | 0.00 | −0.177 | 10.80 | 0.60 |
| ATOM | 2890 | HD1 | TRP | 285 | −10.843 | 24.223 | 13.151 | 1.00 | 0.00 | 0.127 | 0.00 | 0.00 |
| ATOM | 2891 | NE1 | TRP | 285 | −9.559 | 22.549 | 13.739 | 1.00 | 0.00 | −0.292 | 9.00 | −17.40 |
| ATOM | 2892 | HE1 | TRP | 285 | −8.853 | 22.560 | 12.989 | 1.00 | 0.00 | 0.393 | 0.00 | 0.00 |
| ATOM | 2893 | CE3 | TRP | 285 | −10.968 | 21.225 | 16.739 | 1.00 | 0.00 | −0.127 | 10.80 | 0.60 |
| ATOM | 2894 | HE3 | TRP | 285 | −11.826 | 21.448 | 17.372 | 1.00 | 0.00 | 0.127 | 0.00 | 0.00 |
| ATOM | 2895 | CZ2 | TRP | 285 | −8.720 | 20.614 | 15.110 | 1.00 | 0.00 | −0.127 | 10.80 | 0.60 |
| ATOM | 2896 | HZ2 | TRP | 285 | −7.858 | 20.383 | 14.483 | 1.00 | 0.00 | 0.127 | 0.00 | 0.00 |
| ATOM | 2897 | CZ3 | TRP | 285 | −10.100 | 20.171 | 17.059 | 1.00 | 0.00 | −0.127 | 10.80 | 0.60 |
| ATOM | 2898 | HZ3 | TRP | 285 | −10.287 | 19.571 | 17.949 | 1.00 | 0.00 | 0.127 | 0.00 | 0.00 |
| ATOM | 2899 | CH2 | TRP | 285 | −8.994 | 19.879 | 16.247 | 1.00 | 0.00 | −0.127 | 10.80 | 0.60 |
| ATOM | 2900 | HH2 | TRP | 285 | −8.336 | 19.053 | 16.519 | 1.00 | 0.00 | 0.127 | 0.00 | 0.00 |
| ATOM | 2901 | C | TRP | 285 | −11.667 | 25.931 | 16.398 | 1.00 | 0.00 | 0.396 | 9.82 | 4.00 |
| ATOM | 2902 | O | TRP | 285 | −12.179 | 26.840 | 15.748 | 1.00 | 0.00 | −0.396 | 8.17 | −17.40 |
| ATOM | 2903 | N | PRO | 286 | −10.406 | 26.015 | 16.851 | 1.00 | 0.00 | −0.422 | 9.00 | −17.40 |
| ATOM | 2904 | CD | PRO | 286 | −9.649 | 24.891 | 17.443 | 1.00 | 0.00 | 0.105 | 12.77 | 4.00 |
| ATOM | 2905 | HD1 | PRO | 286 | −9.757 | 23.985 | 16.845 | 1.00 | 0.00 | 0.053 | 0.00 | 0.00 |
| ATOM | 2906 | HD2 | PRO | 286 | −9.999 | 24.667 | 18.450 | 1.00 | 0.00 | 0.053 | 0.00 | 0.00 |
| ATOM | 2907 | CA | PRO | 286 | −9.539 | 27.184 | 16.662 | 1.00 | 0.00 | 0.158 | 9.40 | 4.00 |
| ATOM | 2908 | HA | PRO | 286 | −10.106 | 28.070 | 16.944 | 1.00 | 0.00 | 0.053 | 0.00 | 0.00 |
| ATOM | 2909 | CB | PRO | 286 | −6.403 | 26.911 | 17.633 | 1.00 | 0.00 | −0.106 | 12.77 | 4.00 |
| ATOM | 2910 | HB1 | PRO | 286 | −8.677 | 27.170 | 18.655 | 1.00 | 0.00 | 0.053 | 0.00 | 0.00 |
| ATOM | 2911 | HB2 | PRO | 286 | −7.512 | 27.487 | 17.381 | 1.00 | 0.00 | 0.053 | 0.00 | 0.00 |
| ATOM | 2912 | CG | PRO | 286 | −8.207 | 25.412 | 17.439 | 1.00 | 0.00 | −0.106 | 12.77 | 4.00 |
| ATOM | 2913 | HG1 | PRO | 286 | −7.611 | 24.982 | 18.245 | 1.00 | 0.00 | 0.053 | 0.00 | 0.00 |
| ATOM | 2914 | HG2 | PRO | 286 | −7.694 | 25.197 | 16.501 | 1.00 | 0.00 | 0.053 | 0.00 | 0.00 |
| ATOM | 2915 | C | PRO | 286 | −9.026 | 27.413 | 15.241 | 1.00 | 0.00 | 0.396 | 9.82 | 4.00 |
| ATOM | 2916 | O | PRO | 286 | −9.091 | 26.521 | 14.397 | 1.00 | 0.00 | −0.396 | 8.17 | −17.40 |
| ATOM | 2917 | N | LYS | 287S | −8.496 | 28.612 | 14.997 | 1.00 | 0.00 | −0.650 | 9.00 | −17.40 |
| ATOM | 2918 | HN | LYS | 287S | −8.474 | 29.314 | 15.750 | 1.00 | 0.00 | 0.440 | 0.00 | 0.00 |
| ATOM | 2919 | CA | LYS | 287S | −7.942 | 28.956 | 13.681 | 1.00 | 0.00 | 0.158 | 9.40 | 4.00 |
| ATOM | 2920 | HA | LYS | 287S | −8.551 | 28.550 | 12.873 | 1.00 | 0.00 | 0.053 | 0.00 | 0.00 |
| ATOM | 2921 | CB | LYS | 287S | −7.874 | 30.472 | 13.485 | 1.00 | 0.00 | −0.106 | 12.77 | 4.00 |
| ATOM | 2922 | HB1 | LYS | 287S | −7.557 | 30.665 | 12.460 | 1.00 | 0.00 | 0.053 | 0.00 | 0.00 |
| ATOM | 2923 | HB2 | LYS | 287S | −7.151 | 30.871 | 14.196 | 1.00 | 0.00 | 0.053 | 0.00 | 0.00 |
| ATOM | 2924 | CG | LYS | 287S | −9.164 | 31.211 | 13.699 | 1.00 | 0.00 | −0.106 | 12.77 | 4.00 |
| ATOM | 2925 | HG1 | LYS | 287S | −9.025 | 32.279 | 13.536 | 1.00 | 0.00 | 0.053 | 0.00 | 0.00 |
| ATOM | 2926 | HG2 | LYS | 287S | −9.526 | 31.063 | 14.716 | 1.00 | 0.00 | 0.053 | 0.00 | 0.00 |
| ATOM | 2927 | CD | LYS | 287S | −10.225 | 30.718 | 12.740 | 1.00 | 0.00 | −0.106 | 12.77 | 4.00 |
| ATOM | 2928 | HD1 | LYS | 287S | −10.404 | 29.650 | 12.869 | 1.00 | 0.00 | 0.053 | 0.00 | 0.00 |
| ATOM | 2929 | HD2 | LYS | 287S | −9.924 | 30.883 | 11.705 | 1.00 | 0.00 | 0.053 | 0.00 | 0.00 |
| ATOM | 2930 | CE | LYS | 287S | −11.542 | 31.442 | 12.964 | 1.00 | 0.00 | 0.099 | 12.77 | 4.00 |
| ATOM | 2931 | HE1 | LYS | 287S | −11.408 | 32.515 | 12.833 | 1.00 | 0.00 | 0.053 | 0.00 | 0.00 |
| ATOM | 2932 | HE2 | LYS | 287S | −11.907 | 31.255 | 13.974 | 1.00 | 0.00 | 0.053 | 0.00 | 0.00 |
| ATOM | 2933 | NZ | LYS | 287S | −12.561 | 30.966 | 11.993 | 1.00 | 0.00 | −0.045 | 13.25 | −39.20 |
| ATOM | 2934 | HZ1 | LYS | 287S | −13.448 | 31.463 | 12.154 | 1.00 | 0.00 | 0.280 | 0.00 | 0.00 |
| ATOM | 2935 | HZ2 | LYS | 287S | −12.234 | 31.150 | 11.033 | 1.00 | 0.00 | 0.280 | 0.00 | 0.00 |
| ATOM | 2936 | HZ3 | LYS | 287S | −12.707 | 29.954 | 12.117 | 1.00 | 0.00 | 0.280 | 0.00 | 0.00 |
| ATOM | 2937 | C | LYS | 287S | −6.524 | 28.402 | 13.508 | 1.00 | 0.00 | 0.396 | 9.82 | 4.00 |
| ATOM | 2938 | O | LYS | 287S | 6.046 | 28.287 | 12.366 | 1.00 | 0.00 | −0.396 | 8.17 | −17.40 |
| ATOM | 2939 | N | ALA | 288 | −5.852 | 28.066 | 14.617 | 1.00 | 0.00 | −0.650 | 9.00 | −17.40 |
| ATOM | 2940 | HN | ALA | 288 | −6.304 | 28.170 | 15.536 | 1.00 | 0.00 | 0.440 | 0.00 | 0.00 |
| ATOM | 2941 | CA | ALA | 288 | −4.468 | 27.546 | 14.539 | 1.00 | 0.00 | 0.158 | 9.40 | 4.00 |
| ATOM | 2942 | HA | ALA | 288 | −4.330 | 26.784 | 13.771 | 1.00 | 0.00 | 0.053 | 0.00 | 0.00 |
| ATOM | 2943 | CB | ALA | 288 | −3.500 | 28.686 | 14.162 | 1.00 | 0.00 | −0.159 | 16.15 | 4.00 |
| ATOM | 2944 | HB1 | ALA | 288 | −2.483 | 28.296 | 14.106 | 1.00 | 0.00 | 0.053 | 0.00 | 0.00 |
| ATOM | 2945 | HB2 | ALA | 288 | −3.783 | 29.099 | 13.194 | 1.00 | 0.00 | 0.053 | 0.00 | 0.00 |
| ATOM | 2946 | HB3 | ALA | 288 | −3.547 | 29.469 | 14.918 | 1.00 | 0.00 | 0.053 | 0.00 | 0.00 |
| ATOM | 2947 | C | ALA | 288 | −3.986 | 26.896 | 15.831 | 1.00 | 0.00 | 0.396 | 9.82 | 4.00 |
| ATOM | 2948 | O | ALA | 288 | −4.549 | 27.138 | 16.908 | 1.00 | 0.00 | −0.396 | 8.17 | −17.40 |
| ATOM | 2949 | N | PHE | 289 | −2.902 | 26.123 | 15.720 | 1.00 | 0.00 | −0.650 | 9.00 | −17.40 |
| ATOM | 2950 | HN | PHE | 289 | −2.470 | 26.018 | 14.790 | 1.00 | 0.00 | 0.440 | 0.00 | 0.00 |
| ATOM | 2951 | CA | PHE | 289 | −2.294 | 25.416 | 16.852 | 1.00 | 0.00 | 0.158 | 9.40 | 4.00 |
| ATOM | 2952 | HA | PHE | 289 | −1.516 | 24.738 | 16.499 | 1.00 | 0.00 | 0.053 | 0.00 | 0.00 |
| ATOM | 2953 | CB | PHE | 289 | −1.628 | 26.420 | 17.793 | 1.00 | 0.00 | −0.106 | 12.77 | 4.00 |
| ATOM | 2954 | HB1 | PHE | 289 | −0.983 | 25.949 | 18.535 | 1.00 | 0.00 | 0.053 | 0.00 | 0.00 |
| ATOM | 2955 | HB2 | PHE | 289 | −2.343 | 27.014 | 18.361 | 1.00 | 0.00 | 0.053 | 0.00 | 0.00 |
| ATOM | 2956 | CG | PHE | 289 | −0.758 | 27.413 | 17.077 | 1.00 | 0.00 | 0.000 | 7.26 | 0.60 |
| ATOM | 2957 | CD1 | PHE | 289 | −1.173 | 28.730 | 16.908 | 1.00 | 0.00 | −0.127 | 10.80 | 0.60 |
| ATOM | 2958 | HD1 | PHE | 289 | −2.128 | 29.049 | 17.323 | 1.00 | 0.00 | 0.127 | 0.00 | 0.00 |
| ATOM | 2959 | CD2 | PHE | 289 | −0.469 | 27.016 | 16.541 | 1.00 | 0.00 | −0.127 | 10.80 | 0.60 |
| ATOM | 2960 | HD2 | PHE | 289 | 0.806 | 25.987 | 16.668 | 1.00 | 0.00 | 0.127 | 0.00 | 0.00 |
| ATOM | 2961 | CE1 | PHE | 289 | −0.381 | 29.652 | 16.212 | 1.00 | 0.00 | −0.127 | 10.80 | 0.60 |
| ATOM | 2962 | HE1 | PHE | 289 | −0.716 | 30.681 | 16.087 | 1.00 | 0.00 | 0.127 | 0.00 | 0.00 |
| ATOM | 2963 | CE2 | PHE | 289 | 1.261 | 27.918 | 15.849 | 1.00 | 0.00 | −0.127 | 10.80 | 0.60 |
| ATOM | 2964 | HE2 | PHE | 289 | 2.216 | 27.596 | 15.433 | 1.00 | 0.00 | 0.127 | 0.00 | 0.00 |
| ATOM | 2965 | CZ | PHE | 289 | 0.835 | 29.243 | 15.683 | 1.00 | 0.00 | −0.127 | 10.80 | 0.60 |
| ATOM | 2966 | HZ | PHE | 289 | 1.458 | 29.952 | 15.138 | 1.00 | 0.00 | 0.127 | 0.00 | 0.00 |
| ATOM | 2967 | C | PHE | 289 | −3.344 | 24.609 | 17.592 | 1.00 | 0.00 | 0.396 | 9.82 | 4.00 |
| ATOM | 2968 | O | PHE | 289 | −3.559 | 24.779 | 18.808 | 1.00 | 0.00 | −0.396 | 8.17 | −17.40 |
| ATOM | 2969 | N | TRP | 290 | −3.978 | 23.703 | 16.855 | 1.00 | 0.00 | −0.650 | 9.00 | −17.40 |
| ATOM | 2970 | HN | TRP | 290 | −3.697 | 23.566 | 15.873 | 1.00 | 0.00 | 0.440 | 0.00 | 0.00 |
| ATOM | 2971 | CA | TRP | 290 | −5.055 | 22.903 | 17.402 | 1.00 | 0.00 | 0.158 | 9.40 | 4.00 |
| ATOM | 2972 | HA | TRP | 290 | −5.838 | 23.578 | 17.746 | 1.00 | 0.00 | 0.053 | 0.00 | 0.00 |
| ATOM | 2973 | CB | TRP | 290 | −5.722 | 22.072 | 16.295 | 1.00 | 0.00 | −0.106 | 12.77 | 4.00 |
| ATOM | 2974 | HB1 | TRP | 290 | −5.908 | 22.723 | 15.441 | 1.00 | 0.00 | 0.053 | 0.00 | 0.00 |
| ATOM | 2975 | HB2 | TRP | 290 | −6.659 | 21.672 | 16.682 | 1.00 | 0.00 | 0.053 | 0.00 | 0.00 |
| ATOM | 2976 | CG | TRP | 290 | −4.909 | 20.939 | 15.827 | 1.00 | 0.00 | 0.000 | 7.26 | 0.60 |
| ATOM | 2977 | CD2 | TRP | 290 | −4.821 | 19.657 | 16.445 | 1.00 | 0.00 | 0.000 | 6.80 | 0.60 |
| ATOM | 2978 | CE2 | TSP | 290 | −3.800 | 18.942 | 15.783 | 1.00 | 0.00 | −0.050 | 6.80 | 0.60 |
| ATOM | 2979 | CD1 | TRP | 290 | −3.979 | 20.950 | 14.829 | 1.00 | 0.00 | −0.177 | 10.80 | 0.60 |
| ATOM | 2980 | HD1 | TRP | 290 | −3.797 | 21.787 | 14.155 | 1.00 | 0.00 | 0.127 | 0.00 | 0.00 |
| ATOM | 2981 | NE1 | TRP | 290 | −3.303 | 19.751 | 14.796 | 1.00 | 0.00 | −0.292 | 9.00 | −17.40 |
| ATOM | 2982 | HE1 | TRP | 290 | −2.549 | 19.503 | 14.139 | 1.00 | 0.00 | 0.393 | 0.00 | 0.00 |
| ATOM | 2983 | CE3 | TRP | 290 | −5.500 | 19.044 | 17.509 | 1.00 | 0.00 | −0.127 | 10.80 | 0.60 |
| ATOM | 2984 | HE3 | TRP | 290 | −6.297 | 19.567 | 18.035 | 1.00 | 0.00 | 0.127 | 0.00 | 0.00 |
| ATOM | 2985 | CZ2 | TRP | 290 | −3.433 | 17.639 | 16.154 | 1.00 | 0.00 | −0.127 | 10.80 | 0.60 |
| ATOM | 2986 | HZ2 | TRP | 290 | −2.642 | 17.102 | 15.628 | 1.00 | 0.00 | 0.127 | 0.00 | 0.00 |
| ATOM | 2987 | CZ3 | TRP | 290 | −5.133 | 17.753 | 17.878 | 1.00 | 0.00 | −0.127 | 10.80 | 0.60 |
| ATOM | 2988 | HZ3 | TRP | 290 | −5.648 | 17.263 | 18.704 | 1.00 | 0.00 | 0.127 | 0.00 | 0.00 |
| ATOM | 2989 | CH2 | TRP | 290 | −4.104 | 17.068 | 17.199 | 1.00 | 0.00 | −0.127 | 10.80 | 0.60 |
| ATOM | 2990 | HH2 | TRP | 290 | −3.837 | 16.059 | 17.515 | 1.00 | 0.00 | 0.127 | 0.00 | 0.00 |
| ATOM | 2991 | C | TRP | 290 | −4.664 | 22.017 | 18.582 | 1.00 | 0.00 | 0.396 | 9.82 | 4.00 |
| ATOM | 2992 | O | TRP | 290 | −5.436 | 21.909 | 19.538 | 1.00 | 0.00 | −0.396 | 8.17 | −17.40 |
| ATOM | 2993 | N | ASP | 291P | −3.473 | 21.414 | 18.547 | 1.00 | 0.00 | −0.650 | 9.00 | −17.40 |
| ATOM | 2994 | HN | ASP | 291P | −2.848 | 21.560 | 17.740 | 1.00 | 0.00 | 0.440 | 0.00 | 0.00 |
| ATOM | 2995 | CA | ASP | 291P | −3.049 | 20.547 | 19.646 | 1.00 | 0.00 | 0.158 | 9.40 | 4.00 |
| ATOM | 2996 | HA | ASP | 291P | −3.829 | 19.805 | 19.817 | 1.00 | 0.00 | 0.053 | 0.00 | 0.00 |
| ATOM | 2997 | CB | ASP | 291P | −1.822 | 19.690 | 19.241 | 1.00 | 0.00 | −0.336 | 12.77 | 4.00 |
| ATOM | 2998 | HB1 | ASP | 291P | −2.050 | 18.944 | 18.479 | 1.00 | 0.00 | 0.053 | 0.00 | 0.00 |
| ATOM | 2999 | HB2 | ASP | 291P | −1.392 | 19.132 | 20.073 | 1.00 | 0.00 | 0.053 | 0.00 | 0.00 |
| ATOM | 3000 | CG | ASP | 291P | −0.681 | 20.510 | 18.673 | 1.00 | 0.00 | 0.297 | 9.82 | 4.00 |
| ATOM | 3001 | OD1 | ASP | 291P | 0.456 | 20.000 | 18.639 | 1.00 | 0.00 | −0.534 | 8.17 | −18.95 |
| ATOM | 3002 | OD2 | ASP | 291P | −0.908 | 21.653 | 18.247 | 1.00 | 0.00 | −0.534 | 8.17 | −18.95 |
| ATOM | 3003 | C | ASP | 291P | −2.805 | 21.285 | 20.974 | 1.00 | 0.00 | 0.396 | 9.82 | 4.00 |
| ATOM | 3004 | O | ASP | 291P | −3.192 | 20.781 | 22.028 | 1.00 | 0.00 | −0.396 | 8.17 | −17.40 |
| ATOM | 3005 | N | ASP | 292P | −2.180 | 22.461 | 20.937 | 1.00 | 0.00 | −0.650 | 9.00 | −17.40 |
| ATOM | 3006 | HN | ASP | 292P | −1.852 | 22.833 | 20.034 | 1.00 | 0.00 | 0.440 | 0.00 | 0.00 |
| ATOM | 3007 | CA | ASP | 292P | −1.951 | 23.235 | 22.167 | 1.00 | 0.00 | 0.158 | 9.40 | 4.00 |
| ATOM | 3008 | HA | ASP | 292P | −1.505 | 22.602 | 22.935 | 1.00 | 0.00 | 0.053 | 0.00 | 0.00 |
| ATOM | 3009 | CB | ASP | 292P | −0.960 | 24.384 | 21.932 | 1.00 | 0.00 | −0.336 | 12.77 | 4.00 |
| ATOM | 3010 | HB1 | ASP | 292P | −1.069 | 25.178 | 22.670 | 1.00 | 0.00 | 0.053 | 0.00 | 0.00 |
| ATOM | 3011 | HB2 | ASP | 292P | −1.091 | 24.846 | 20.953 | 1.00 | 0.00 | 0.053 | 0.00 | 0.00 |
| ATOM | 3012 | CG | ASP | 292P | 0.485 | 23.918 | 22.004 | 1.00 | 0.00 | 0.297 | 9.82 | 4.00 |
| ATOM | 3013 | OD1 | ASP | 292P | 0.715 | 22.720 | 22.323 | 1.00 | 0.00 | −0.534 | 8.17 | −18.95 |
| ATOM | 3014 | OD2 | ASP | 292P | 1.392 | 24.733 | 21.758 | 1.00 | 0.00 | −0.534 | 8.17 | −18.95 |
| ATOM | 3015 | C | ASP | 292P | −3.265 | 23.788 | 22.696 | 1.00 | 0.00 | 0.396 | 9.82 | 4.00 |
| ATOM | 3016 | O | ASP | 292P | −3.420 | 24.012 | 23.913 | 1.00 | 0.00 | −0.396 | 8.17 | −17.40 |
| ATOM | 3017 | N | TRP | 293 | −4.207 | 24.001 | 21.777 | 1.00 | 0.00 | −0.650 | 9.00 | −17.40 |
| ATOM | 3018 | HN | TRP | 293 | −3.987 | 23.808 | 20.789 | 1.00 | 0.00 | 0.440 | 0.00 | 0.00 |
| ATOM | 3019 | CA | TRP | 293 | −5.551 | 24.501 | 22.120 | 1.00 | 0.00 | 0.158 | 9.40 | 4.00 |
| ATOM | 3020 | HA | TRP | 293 | −5.457 | 25.391 | 22.742 | 1.00 | 0.00 | 0.053 | 0.00 | 0.00 |
| ATOM | 3021 | CB | TRP | 293 | −6.296 | 24.863 | 20.834 | 1.00 | 0.00 | −0.106 | 12.77 | 4.00 |
| ATOM | 3022 | HB1 | TRP | 293 | −6.241 | 24.005 | 20.163 | 1.00 | 0.00 | 0.053 | 0.00 | 0.00 |
| ATOM | 3023 | HB2 | TRP | 293 | −5.805 | 25.733 | 20.397 | 1.00 | 0.00 | 0.053 | 0.00 | 0.00 |
| ATOM | 3024 | CG | TRP | 293 | −7.737 | 25.206 | 20.986 | 1.00 | 0.00 | 0.000 | 7.26 | 0.60 |
| ATOM | 3025 | CD2 | TRP | 293 | −8.854 | 24.339 | 20.738 | 1.00 | 0.00 | 0.000 | 6.80 | 0.60 |
| ATOM | 3026 | CE2 | TRP | 293 | −10.028 | 25.094 | 20.971 | 1.00 | 0.00 | −0.050 | 6.80 | 0.60 |
| ATOM | 3027 | CD1 | TRP | 293 | −8.263 | 26.413 | 21.353 | 1.00 | 0.00 | −0.177 | 10.80 | 0.60 |
| ATOM | 3028 | HD1 | TRP | 293 | −7.677 | 27.294 | 21.615 | 1.00 | 0.00 | 0.127 | 0.00 | 0.00 |
| ATOM | 3029 | NE1 | TRP | 293 | −9.639 | 26.356 | 21.344 | 1.00 | 0.00 | −0.292 | 9.00 | −17.40 |
| ATOM | 3030 | HE1 | TRP | 293 | −10.272 | 27.133 | 21.579 | 1.00 | 0.00 | 0.393 | 0.00 | 0.00 |
| ATOM | 3031 | CE3 | TRP | 293 | −8.974 | 22.995 | 20.345 | 1.00 | 0.00 | −0.127 | 10.80 | 0.60 |
| ATOM | 3032 | HE3 | TRP | 293 | −8.086 | 22.390 | 20.161 | 1.00 | 0.00 | 0.127 | 0.00 | 0.00 |
| ATOM | 3033 | CZ2 | TRP | 293 | −11.308 | 24.554 | 20.820 | 1.00 | 0.00 | −0.127 | 10.80 | 0.60 |
| ATOM | 3034 | HZ2 | TRP | 293 | −12.199 | 25.153 | 21.003 | 1.00 | 0.00 | 0.127 | 0.00 | 0.00 |
| ATOM | 3035 | CZ3 | TRP | 293 | −10.245 | 22.449 | 20.194 | 1.00 | 0.00 | −0.127 | 10.80 | 0.60 |
| ATOM | 3036 | HZ3 | TRP | 293 | −10.353 | 21.407 | 19.890 | 1.00 | 0.00 | 0.127 | 0.00 | 0.00 |
| ATOM | 3037 | CH2 | TRP | 293 | −11.400 | 23.235 | 20.431 | 1.00 | 0.00 | −0.127 | 10.80 | 0.60 |
| ATOM | 3038 | HH2 | TRP | 293 | −12.384 | 22.785 | 20.302 | 1.00 | 0.00 | 0.127 | 0.00 | 0.00 |
| ATOM | 3039 | C | TRP | 293 | −6.318 | 23.415 | 22.888 | 1.00 | 0.00 | 0.396 | 9.82 | 4.00 |
| ATOM | 3040 | O | TRP | 293 | −7.058 | 23.699 | 23.840 | 1.00 | 0.00 | −0.396 | 8.17 | −17.40 |
| ATOM | 3041 | N | MET | 294 | −6.155 | 22.166 | 22.478 | 1.00 | 0.00 | −0.650 | 9.00 | −17.40 |
| ATOM | 3042 | HN | MET | 294 | −5.559 | 21.956 | 21.664 | 1.00 | 0.00 | 0.440 | 0.00 | 0.00 |
| ATOM | 3043 | CA | MET | 294 | −6.835 | 21.087 | 23.200 | 1.00 | 0.00 | 0.158 | 9.40 | 4.00 |
| ATOM | 3044 | HA | MET | 294 | −7.888 | 21.330 | 23.340 | 1.00 | 0.00 | 0.053 | 0.00 | 0.00 |
| ATOM | 3045 | CB | MET | 294 | −6.777 | 19.782 | 22.399 | 1.00 | 0.00 | −0.106 | 12.77 | 4.00 |
| ATOM | 3046 | HB1 | MET | 294 | −6.802 | 18.947 | 23.100 | 1.00 | 0.00 | 0.053 | 0.00 | 0.00 |
| ATOM | 3047 | HB2 | MET | 294 | −5.850 | 19.772 | 21.825 | 1.00 | 0.00 | 0.053 | 0.00 | 0.00 |
| ATOM | 3048 | CG | MET | 294 | −7.941 | 19.600 | 21.412 | 1.00 | 0.00 | −0.041 | 12.77 | 4.00 |
| ATOM | 3049 | HG1 | MET | 294 | −8.018 | 20.448 | 20.732 | 1.00 | 0.00 | 0.053 | 0.00 | 0.00 |
| ATOM | 3050 | HG2 | MET | 294 | −8.892 | 19.508 | 21.936 | 1.00 | 0.00 | 0.053 | 0.00 | 0.00 |
| ATOM | 3051 | SD | MET | 294 | −7.725 | 18.134 | 20.424 | 1.00 | 0.00 | −0.130 | 16.39 | −6.40 |
| ATOM | 3052 | CE | MET | 294 | −8.130 | 16.824 | 21.657 | 1.00 | 0.00 | −0.094 | 16.15 | 4.00 |
| ATOM | 3053 | HE1 | MET | 294 | −8.040 | 15.843 | 21.189 | 1.00 | 0.00 | 0.053 | 0.00 | 0.00 |
| ATOM | 3054 | HE2 | MET | 294 | −7.439 | 16.889 | 22.497 | 1.00 | 0.00 | 0.053 | 0.00 | 0.00 |
| ATOM | 3055 | HE3 | MET | 294 | −9.150 | 16.963 | 22.014 | 1.00 | 0.00 | 0.053 | 0.00 | 0.00 |
| ATOM | 3056 | C | MET | 294 | −6.195 | 20.881 | 24.574 | 1.00 | 0.00 | 0.396 | 9.82 | 4.00 |
| ATOM | 3057 | O | MET | 294 | −6.825 | 20.326 | 25.480 | 1.00 | 0.00 | −0.396 | 8.17 | −17.40 |
| ATOM | 3058 | N | ARG | 295G | −4.950 | 21.329 | 24.733 | 1.00 | 0.00 | −0.650 | 9.00 | −17.40 |
| ATOM | 3059 | HN | ARG | 295G | −4.470 | 21.789 | 23.946 | 1.00 | 0.00 | 0.440 | 0.00 | 0.00 |
| ATOM | 3060 | CA | ARG | 295G | −4.263 | 21.176 | 26.002 | 1.00 | 0.00 | 0.158 | 9.40 | 4.00 |
| ATOM | 3061 | HA | ARG | 295G | −4.388 | 20.185 | 26.439 | 1.00 | 0.00 | 0.053 | 0.00 | 0.00 |
| ATOM | 3062 | CB | ARG | 295G | −2.744 | 21.266 | 25.810 | 1.00 | 0.00 | −0.106 | 12.77 | 4.00 |
| ATOM | 3063 | HB1 | ARG | 295G | −2.221 | 21.504 | 26.736 | 1.00 | 0.00 | 0.053 | 0.00 | 0.00 |
| ATOM | 3064 | HB2 | ARG | 295G | −2.460 | 22.034 | 25.090 | 1.00 | 0.00 | 0.053 | 0.00 | 0.00 |
| ATOM | 3065 | CG | ARG | 295G | −2.140 | 19.952 | 25.299 | 1.00 | 0.00 | −0.106 | 12.77 | 4.00 |
| ATOM | 3066 | HG1 | ARG | 295G | −2.800 | 19.401 | 24.628 | 1.00 | 0.00 | 0.053 | 0.00 | 0.00 |
| ATOM | 3067 | HG2 | ARG | 295G | −1.891 | 19.249 | 26.094 | 1.00 | 0.00 | 0.053 | 0.00 | 0.00 |
| ATOM | 3068 | CD | ARG | 295G | −0.838 | 20.143 | 24.512 | 1.00 | 0.00 | 0.374 | 12.77 | 4.00 |
| ATOM | 3069 | HD1 | ARG | 295G | −0.034 | 20.468 | 25.172 | 1.00 | 0.00 | 0.053 | 0.00 | 0.00 |
| ATOM | 3070 | HD2 | ARG | 295G | −0.967 | 20.894 | 23.733 | 1.00 | 0.00 | 0.053 | 0.00 | 0.00 |
| ATOM | 3071 | NE | ARG | 295G | −0.430 | 18.885 | 23.875 | 1.00 | 0.00 | −0.819 | 9.00 | −24.67 |
| ATOM | 3072 | HE | ARG | 295G | −0.793 | 18.003 | 24.265 | 1.00 | 0.00 | 0.407 | 0.00 | 0.00 |
| ATOM | 3073 | CZ | ARG | 295G | 0.380 | 18.816 | 22.825 | 1.00 | 0.00 | 0.796 | 6.95 | 4.00 |
| ATOM | 3074 | NH1 | ARG | 295G | 0.875 | 19.927 | 22.284 | 1.00 | 0.00 | −0.746 | 9.00 | −24.67 |
| ATOM | 3075 | HH1 | ARG | 295G | 1.502 | 19.867 | 21.469 | 1.00 | 0.00 | 0.407 | 0.00 | 0.00 |
| ATOM | 3076 | HH1 | ARG | 295G | 0.630 | 20.845 | 22.679 | 1.00 | 0.00 | 0.407 | 0.00 | 0.00 |
| ATOM | 3077 | NH2 | ARG | 295G | 0.697 | 17.639 | 22.307 | 1.00 | 0.00 | −0.746 | 9.00 | −24.67 |
| ATOM | 3078 | HH2 | ARG | 295G | 1.325 | 17.587 | 21.492 | 1.00 | 0.00 | 0.407 | 0.00 | 0.00 |
| ATOM | 3079 | HH2 | ARG | 295G | 0.315 | 16.775 | 22.718 | 1.00 | 0.00 | 0.407 | 0.00 | 0.00 |
| ATOM | 3080 | C | ARG | 295G | −4.746 | 22.160 | 27.067 | 1.00 | 0.00 | 0.396 | 9.82 | 4.00 |
| ATOM | 3081 | O | ARG | 295G | −4.470 | 21.983 | 28.255 | 1.00 | 0.00 | −0.396 | 8.17 | −17.40 |
| ATOM | 3082 | N | ARG | 296G | −5.485 | 23.178 | 26.646 | 1.00 | 0.00 | −0.650 | 9.00 | −17.40 |
| ATOM | 3083 | HN | ARG | 296G | −5.682 | 23.276 | 25.639 | 1.00 | 0.00 | 0.440 | 0.00 | 0.00 |
| ATOM | 3084 | CA | ARG | 296G | −6.026 | 24.164 | 27.582 | 1.00 | 0.00 | 0.158 | 9.40 | 4.00 |
| ATOM | 3085 | HA | ARG | 296G | −5.251 | 24.530 | 28.255 | 1.00 | 0.00 | 0.053 | 0.00 | 0.00 |
| ATOM | 3086 | CB | ARG | 296G | −6.596 | 25.359 | 26.826 | 1.00 | 0.00 | −0.106 | 12.77 | 4.00 |
| ATOM | 3087 | HB1 | ARG | 296G | −7.008 | 26.122 | 27.485 | 1.00 | 0.00 | 0.053 | 0.00 | 0.00 |
| ATOM | 3088 | HB2 | ARG | 296G | −7.404 | 25.087 | 26.146 | 1.00 | 0.00 | 0.053 | 0.00 | 0.00 |
| ATOM | 3089 | CG | ARG | 296G | −5.575 | 26.059 | 25.983 | 1.00 | 0.00 | −0.106 | 12.77 | 4.00 |
| ATOM | 3090 | HG1 | ARG | 296G | −5.186 | 25.362 | 25.240 | 1.00 | 0.00 | 0.053 | 0.00 | 0.00 |
| ATOM | 3091 | HG2 | ARG | 296G | −4.763 | 26.408 | 26.621 | 1.00 | 0.00 | 0.053 | 0.00 | 0.00 |
| ATOM | 3092 | CD | ARG | 296G | −6.219 | 27.222 | 25.299 | 1.00 | 0.00 | 0.374 | 12.77 | 4.00 |
| ATOM | 3093 | HD1 | ARG | 296G | −6.638 | 27.931 | 26.012 | 1.00 | 0.00 | 0.053 | 0.00 | 0.00 |
| ATOM | 3094 | HD2 | ARG | 296G | −7.033 | 26.909 | 24.645 | 1.00 | 0.00 | 0.053 | 0.00 | 0.00 |
| ATOM | 3095 | NE | ARG | 296G | −5.276 | 27.956 | 24.477 | 1.00 | 0.00 | −0.819 | 9.00 | −24.67 |
| ATOM | 3096 | HE | ARG | 296G | −4.284 | 27.676 | 24.497 | 1.00 | 0.00 | 0.407 | 0.00 | 0.00 |
| ATOM | 3097 | CZ | ARG | 296G | −5.632 | 28.974 | 23.698 | 1.00 | 0.00 | 0.796 | 6.95 | 4.00 |
| ATOM | 3098 | NH1 | ARG | 296G | −6.911 | 29.356 | 23.646 | 1.00 | 0.00 | −0.746 | 9.00 | −24.67 |
| ATOM | 3099 | HH1 | ARG | 296G | −7.189 | 30.143 | 23.043 | 1.00 | 0.00 | 0.407 | 0.00 | 0.00 |
| ATOM | 3100 | HH1 | ARG | 296G | −7.619 | 28.862 | 24.208 | 1.00 | 0.00 | 0.407 | 0.00 | 0.00 |
| ATOM | 3101 | NH2 | ARG | 296G | −4.715 | 29.618 | 22.987 | 1.00 | 0.00 | −0.746 | 9.00 | −24.67 |
| ATOM | 3102 | HH2 | ARG | 296G | −4.990 | 30.406 | 22.384 | 1.00 | 0.00 | 0.407 | 0.00 | 0.00 |
| ATOM | 3103 | HH2 | ARG | 296G | −3.727 | 29.329 | 23.038 | 1.00 | 0.00 | 0.407 | 0.00 | 0.00 |
| ATOM | 3104 | C | ARG | 296G | −7.141 | 23.557 | 28.436 | 1.00 | 0.00 | 0.396 | 9.82 | 4.00 |
| ATOM | 3105 | O | ARG | 296G | −7.880 | 22.667 | 27.986 | 1.00 | 0.00 | −0.396 | 8.17 | −17.40 |
| ATOM | 3106 | N | PRO | 297 | −7.305 | 24.059 | 29.670 | 1.00 | 0.00 | −0.422 | 9.00 | −17.40 |
| ATOM | 3107 | CD | PRO | 297 | −6.485 | 25.036 | 30.414 | 1.00 | 0.00 | 0.105 | 12.77 | 4.00 |
| ATOM | 3108 | HD1 | PRO | 297 | −6.254 | 25.906 | 29.800 | 1.00 | 0.00 | 0.053 | 0.00 | 0.00 |
| ATOM | 3109 | HD2 | PRO | 297 | −5.539 | 24.596 | 30.732 | 1.00 | 0.00 | 0.053 | 0.00 | 0.00 |
| ATOM | 3110 | CA | PRO | 297 | −8.367 | 23.477 | 30.501 | 1.00 | 0.00 | 0.158 | 9.40 | 4.00 |
| ATOM | 3111 | HA | PRO | 297 | −8.203 | 22.400 | 30.554 | 1.00 | 0.00 | 0.053 | 0.00 | 0.00 |
| ATOM | 3112 | CB | PRO | 297 | −8.107 | 24.085 | 31.892 | 1.00 | 0.00 | −0.106 | 12.77 | 4.00 |
| ATOM | 3113 | HB1 | PRO | 297 | −7.495 | 23.339 | 32.401 | 1.00 | 0.00 | 0.053 | 0.00 | 0.00 |
| ATOM | 3114 | HB2 | PRO | 297 | −9.101 | 24.213 | 32.319 | 1.00 | 0.00 | 0.053 | 0.00 | 0.00 |
| ATOM | 3115 | CG | PRO | 297 | −7.382 | 25.391 | 31.601 | 1.00 | 0.00 | −0.106 | 12.77 | 4.00 |
| ATOM | 3116 | HG1 | PRO | 297 | −6.802 | 25.717 | 32.464 | 1.00 | 0.00 | 0.053 | 0.00 | 0.00 |
| ATOM | 3117 | HG2 | PRO | 297 | −8.087 | 26.184 | 31.353 | 1.00 | 0.00 | 0.053 | 0.00 | 0.00 |
| ATOM | 3118 | C | PRO | 297 | −9.792 | 23.699 | 29.982 | 1.00 | 0.00 | 0.396 | 9.82 | 4.00 |
| ATOM | 3119 | O | PRO | 297 | −10.681 | 22.870 | 30.227 | 1.00 | 0.00 | −0.396 | 8.17 | −17.40 |
| ATOM | 3120 | N | GLU | 298 | −10.013 | 24.790 | 29.252 | 1.00 | 0.00 | −0.650 | 9.00 | −17.40 |
| ATOM | 3121 | HN | GLU | 298 | −9.240 | 25.444 | 29.062 | 1.00 | 0.00 | 0.440 | 0.00 | 0.00 |
| ATOM | 3122 | CA | GLU | 298 | −11.341 | 25.064 | 28.720 | 1.00 | 0.00 | 0.158 | 9.40 | 4.00 |
| ATOM | 3123 | HA | GLU | 298 | −12.060 | 25.131 | 29.536 | 1.00 | 0.00 | 0.053 | 0.00 | 0.00 |
| ATOM | 3124 | CB | GLU | 298 | −11.371 | 26.396 | 27.951 | 1.00 | 0.00 | −0.106 | 12.77 | 4.00 |
| ATOM | 3125 | HB1 | GLU | 298 | −12.354 | 26.494 | 27.491 | 1.00 | 0.00 | 0.053 | 0.00 | 0.00 |
| ATOM | 3126 | HB2 | GLU | 298 | −10.586 | 26.362 | 27.194 | 1.00 | 0.00 | 0.053 | 0.00 | 0.00 |
| ATOM | 3127 | CG | GLU | 298 | −11.138 | 27.656 | 28.780 | 1.00 | 0.00 | −0.106 | 12.77 | 4.00 |
| ATOM | 3128 | HG1 | GLU | 298 | −11.752 | 27.701 | 29.679 | 1.00 | 0.00 | 0.053 | 0.00 | 0.00 |
| ATOM | 3129 | HG2 | GLU | 298 | −11.352 | 28.576 | 28.237 | 1.00 | 0.00 | 0.053 | 0.00 | 0.00 |
| ATOM | 3130 | CD | GLU | 298 | −9.699 | 27.802 | 29.268 | 1.00 | 0.00 | 0.399 | 9.82 | 4.00 |
| ATOM | 3131 | OE1 | GLU | 298 | −8.803 | 27.167 | 28.681 | 1.00 | 0.00 | −0.396 | 8.17 | −18.95 |
| ATOM | 3132 | OE2 | GLU | 298 | −9.468 | 28.568 | 30.236 | 1.00 | 0.00 | −0.427 | 8.17 | −18.95 |
| ATOM | 3133 | HE2 | GLU | 298 | −8.458 | 28.570 | 30.439 | 1.00 | 0.00 | 0.424 | 0.00 | 0.00 |
| ATOM | 3134 | C | GLU | 298 | −11.765 | 23.938 | 27.771 | 1.00 | 0.00 | 0.396 | 9.82 | 4.00 |
| ATOM | 3135 | O | GLU | 298 | −12.947 | 23.729 | 27.537 | 1.00 | 0.00 | −0.396 | 8.17 | −17.40 |
| ATOM | 3136 | N | GLN | 299 | −10.792 | 23.226 | 27.216 | 1.00 | 0.00 | −0.650 | 9.00 | −17.40 |
| ATOM | 3137 | HN | GLN | 299 | −9.812 | 23.453 | 27.437 | 1.00 | 0.00 | 0.440 | 0.00 | 0.00 |
| ATOM | 3138 | CA | GLN | 299 | −11.081 | 22.127 | 26.298 | 1.00 | 0.00 | 0.158 | 9.40 | 4.00 |
| ATOM | 3139 | HA | GLN | 299 | −12.084 | 22.214 | 25.880 | 1.00 | 0.00 | 0.053 | 0.00 | 0.00 |
| ATOM | 3140 | CB | GLN | 299 | −10.079 | 22.119 | 25.141 | 1.00 | 0.00 | −0.106 | 12.77 | 4.00 |
| ATOM | 3141 | HB1 | GLN | 299 | −10.013 | 21.098 | 24.763 | 1.00 | 0.00 | 0.053 | 0.00 | 0.00 |
| ATOM | 3142 | HB2 | GLN | 299 | −9.118 | 22.455 | 25.530 | 1.00 | 0.00 | 0.053 | 0.00 | 0.00 |
| ATOM | 3143 | CG | GLN | 299 | −10.417 | 23.017 | 23.945 | 1.00 | 0.00 | −0.106 | 12.77 | 4.00 |
| ATOM | 3144 | HG1 | GLN | 299 | −11.321 | 22.717 | 23.415 | 1.00 | 0.00 | 0.053 | 0.00 | 0.00 |
| ATOM | 3145 | HG2 | GLN | 299 | −9.638 | 23.041 | 23.182 | 1.00 | 0.00 | 0.053 | 0.00 | 0.00 |
| ATOM | 3146 | CD | GLN | 299 | −10.647 | 24.484 | 24.323 | 1.00 | 0.00 | 0.396 | 9.82 | 4.00 |
| ATOM | 3147 | OE1 | GLN | 299 | −11.778 | 24.895 | 24.584 | 1.00 | 0.00 | −0.396 | 8.17 | −17.40 |
| ATOM | 3148 | NE2 | GLN | 299 | −9.568 | 25.274 | 24.357 | 1.00 | 0.00 | −0.879 | 13.25 | −17.40 |
| ATOM | 3149 | HE2 | GLN | 299 | −8.640 | 24.888 | 24.131 | 1.00 | 0.00 | 0.440 | 0.00 | 0.00 |
| ATOM | 3150 | HE2 | GLN | 299 | −9.665 | 26.268 | 24.609 | 1.00 | 0.00 | 0.440 | 0.00 | 0.00 |
| ATOM | 3151 | C | GLN | 299 | −10.991 | 20.785 | 27.001 | 1.00 | 0.00 | 0.396 | 9.82 | 4.00 |
| ATOM | 3152 | O | GLN | 299 | −11.876 | 19.947 | 26.883 | 1.00 | 0.00 | −0.396 | 8.17 | −17.40 |
| ATOM | 3153 | N | ARG | 300G | −9.891 | 20.596 | 27.723 | 1.00 | 0.00 | −0.650 | 9.00 | −17.40 |
| ATOM | 3154 | HN | ARG | 300G | −9.207 | 21.363 | 27.794 | 1.00 | 0.00 | 0.440 | 0.00 | 0.00 |
| ATOM | 3155 | CA | ARG | 300G | −9.614 | 19.345 | 28.418 | 1.00 | 0.00 | 0.158 | 9.40 | 4.00 |
| ATOM | 3156 | HA | ARG | 300G | −9.620 | 18.472 | 27.764 | 1.00 | 0.00 | 0.053 | 0.00 | 0.00 |
| ATOM | 3157 | CB | ARG | 300G | −8.206 | 19.406 | 29.013 | 1.00 | 0.00 | −0.106 | 12.77 | 4.00 |
| ATOM | 3158 | HB1 | ARG | 300G | −8.217 | 20.106 | 29.847 | 1.00 | 0.00 | 0.053 | 0.00 | 0.00 |
| ATOM | 3159 | HB2 | ARG | 300G | −7.520 | 19.745 | 28.236 | 1.00 | 0.00 | 0.053 | 0.00 | 0.00 |
| ATOM | 3160 | CG | ARG | 300G | −7.690 | 18.075 | 29.535 | 1.00 | 0.00 | −0.106 | 12.77 | 4.00 |
| ATOM | 3161 | HG1 | ARG | 300G | −7.842 | 17.289 | 28.795 | 1.00 | 0.00 | 0.053 | 0.00 | 0.00 |
| ATOM | 3162 | HG2 | ARG | 300G | −8.214 | 17.793 | 30.448 | 1.00 | 0.00 | 0.053 | 0.00 | 0.00 |
| ATOM | 3163 | CD | ARG | 300G | −6.192 | 18.164 | 29.845 | 1.00 | 0.00 | 0.374 | 12.77 | 4.00 |
| ATOM | 3164 | HD1 | ARG | 300G | −5.720 | 18.784 | 29.083 | 1.00 | 0.00 | 0.053 | 0.00 | 0.00 |
| ATOM | 3165 | HD2 | ARG | 300G | −5.777 | 17.156 | 29.828 | 1.00 | 0.00 | 0.053 | 0.00 | 0.00 |
| ATOM | 3166 | NE | ARG | 300G | −5.887 | 18.751 | 31.151 | 1.00 | 0.00 | −0.819 | 9.00 | −24.67 |
| ATOM | 3167 | HE | ARG | 300G | −5.490 | 19.701 | 31.169 | 1.00 | 0.00 | 0.407 | 0.00 | 0.00 |
| ATOM | 3168 | CZ | ARG | 300G | −6.088 | 18.139 | 32.319 | 1.00 | 0.00 | 0.796 | 6.95 | 4.00 |
| ATOM | 3169 | NH1 | ARG | 300G | −6.606 | 16.908 | 32.369 | 1.00 | 0.00 | −0.746 | 9.00 | −24.67 |
| ATOM | 3170 | HH1 | ARG | 300G | −6.755 | 16.448 | 33.278 | 1.00 | 0.00 | 0.407 | 0.00 | 0.00 |
| ATOM | 3171 | HH1 | ARG | 300G | −6.856 | 16.418 | 31.497 | 1.00 | 0.00 | 0.407 | 0.00 | 0.00 |
| ATOM | 3172 | NH2 | ARG | 300G | −5.749 | 18.750 | 33.449 | 1.00 | 0.00 | −0.746 | 9.00 | −24.67 |
| ATOM | 3173 | HH2 | ARG | 300G | −5.902 | 18.281 | 34.353 | 1.00 | 0.00 | 0.407 | 0.00 | 0.00 |
| ATOM | 3174 | HH2 | ARG | 300G | −5.333 | 19.691 | 33.420 | 1.00 | 0.00 | 0.407 | 0.00 | 0.00 |
| ATOM | 3175 | C | ARG | 300G | −10.612 | 19.025 | 29.511 | 1.00 | 0.00 | 0.396 | 9.82 | 4.00 |
| ATOM | 3176 | O | ARG | 300G | −11.061 | 17.889 | 29.647 | 1.00 | 0.00 | −0.396 | 8.17 | −17.40 |
| ATOM | 3177 | N | LYS | 301S | −10.925 | 20.034 | 30.314 | 1.00 | 0.00 | −0.650 | 9.00 | −17.40 |
| ATOM | 3178 | HN | LYS | 301S | −10.500 | 20.957 | 30.144 | 1.00 | 0.00 | 0.440 | 0.00 | 0.00 |
| ATOM | 3179 | CA | LYS | 301S | −11.847 | 19.877 | 31.424 | 1.00 | 0.00 | 0.158 | 9.40 | 4.00 |
| ATOM | 3180 | HA | LYS | 301S | −11.889 | 20.776 | 32.038 | 1.00 | 0.00 | 0.053 | 0.00 | 0.00 |
| ATOM | 3181 | CB | LYS | 301S | −13.257 | 19.668 | 30.882 | 1.00 | 0.00 | −0.106 | 12.77 | 4.00 |
| ATOM | 3182 | HB1 | LYS | 301S | −13.987 | 19.441 | 31.659 | 1.00 | 0.00 | 0.053 | 0.00 | 0.00 |
| ATOM | 3183 | HB2 | LYS | 301S | −13.326 | 18.845 | 30.169 | 1.00 | 0.00 | 0.053 | 0.00 | 0.00 |
| ATOM | 3184 | CG | LYS | 301S | −13.810 | 20.894 | 30.146 | 1.00 | 0.00 | −0.106 | 12.77 | 4.00 |
| ATOM | 3185 | HG1 | LYS | 301S | −13.086 | 21.314 | 29.447 | 1.00 | 0.00 | 0.053 | 0.00 | 0.00 |
| ATOM | 3186 | HG2 | LYS | 301S | −14.084 | 21.695 | 30.832 | 1.00 | 0.00 | 0.053 | 0.00 | 0.00 |
| ATOM | 3187 | CD | LYS | 301S | −15.057 | 20.563 | 29.338 | 1.00 | 0.00 | −0.106 | 12.77 | 4.00 |
| ATOM | 3188 | HD1 | LYS | 301S | −15.869 | 20.192 | 29.963 | 1.00 | 0.00 | 0.053 | 0.00 | 0.00 |
| ATOM | 3189 | HD2 | LYS | 301S | −14.871 | 19.796 | 28.586 | 1.00 | 0.00 | 0.053 | 0.00 | 0.00 |
| ATOM | 3190 | CE | LYS | 301S | −15.604 | 21.777 | 28.592 | 1.00 | 0.00 | 0.099 | 12.77 | 4.00 |
| ATOM | 3191 | HE1 | LYS | 301S | −14.893 | 22.158 | 27.859 | 1.00 | 0.00 | 0.053 | 0.00 | 0.00 |
| ATOM | 3192 | HE2 | LYS | 301S | −15.834 | 22.600 | 29.268 | 1.00 | 0.00 | 0.053 | 0.00 | 0.00 |
| ATOM | 3193 | NZ | LYS | 301S | −16.871 | 21.454 | 27.847 | 1.00 | 0.00 | −0.045 | 13.25 | −39.20 |
| ATOM | 3194 | HZ1 | LYS | 301S | −17.206 | 22.296 | 27.358 | 1.00 | 0.00 | 0.280 | 0.00 | 0.00 |
| ATOM | 3195 | HZ2 | LYS | 301S | −16.685 | 20.710 | 27.159 | 1.00 | 0.00 | 0.280 | 0.00 | 0.00 |
| ATOM | 3196 | HZ3 | LYS | 301S | −17.589 | 21.134 | 28.512 | 1.00 | 0.00 | 0.280 | 0.00 | 0.00 |
| ATOM | 3197 | C | LYS | 301S | −11.431 | 18.712 | 32.342 | 1.00 | 0.00 | 0.396 | 9.82 | 4.00 |
| ATOM | 3198 | O | LYS | 301S | −12.215 | 17.819 | 32.634 | 1.00 | 0.00 | −0.396 | 8.17 | −17.40 |
| ATOM | 3199 | N | GLY | 302 | −10.171 | 18.719 | 32.759 | 1.00 | 0.00 | −0.650 | 9.00 | −17.40 |
| ATOM | 3200 | HN | GLY | 302 | −9.532 | 19.457 | 32.431 | 1.00 | 0.00 | 0.440 | 0.00 | 0.00 |
| ATOM | 3201 | CA | GLY | 302 | −9.678 | 17.699 | 33.672 | 1.00 | 0.00 | 0.105 | 9.40 | 4.00 |
| ATOM | 3202 | HA1 | GLY | 302 | −10.336 | 17.696 | 34.540 | 1.00 | 0.00 | 0.053 | 0.00 | 0.00 |
| ATOM | 3203 | HA2 | GLY | 302 | −8.658 | 17.973 | 33.942 | 1.00 | 0.00 | 0.053 | 0.00 | 0.00 |
| ATOM | 3204 | C | GLY | 302 | −9.614 | 16.263 | 33.188 | 1.00 | 0.00 | 0.396 | 9.82 | 4.00 |
| ATOM | 3205 | O | GLY | 302 | −9.218 | 15.381 | 33.945 | 1.00 | 0.00 | −0.396 | 8.17 | −17.40 |
| ATOM | 3206 | N | ARG | 303G | −9.966 | 16.026 | 31.928 | 1.00 | 0.00 | −0.650 | 9.00 | −17.40 |
| ATOM | 3207 | HN | ARG | 303G | −10.247 | 16.814 | 31.328 | 1.00 | 0.00 | 0.440 | 0.00 | 0.00 |
| ATOM | 3208 | CA | ARG | 303G | −9.963 | 14.683 | 31.378 | 1.00 | 0.00 | 0.158 | 9.40 | 4.00 |
| ATOM | 3209 | HA | ARG | 303G | −10.342 | 13.947 | 32.087 | 1.00 | 0.00 | 0.053 | 0.00 | 0.00 |
| ATOM | 3210 | CB | ARG | 303G | −10.920 | 14.619 | 30.191 | 1.00 | 0.00 | −0.106 | 12.77 | 4.00 |
| ATOM | 3211 | HB1 | ARG | 303G | −10.860 | 13.673 | 29.652 | 1.00 | 0.00 | 0.053 | 0.00 | 0.00 |
| ATOM | 3212 | HB2 | ARG | 303G | −10.728 | 15.398 | 29.453 | 1.00 | 0.00 | 0.053 | 0.00 | 0.00 |
| ATOM | 3213 | CG | ARG | 303G | −12.388 | 14.779 | 30.604 | 1.00 | 0.00 | −0.106 | 12.77 | 4.00 |
| ATOM | 3214 | HG1 | ARG | 303G | −12.549 | 15.676 | 31.201 | 1.00 | 0.00 | 0.053 | 0.00 | 0.00 |
| ATOM | 3215 | HG2 | ARG | 303G | −12.739 | 13.936 | 31.199 | 1.00 | 0.00 | 0.053 | 0.00 | 0.00 |
| ATOM | 3216 | CD | ARG | 303G | −13.316 | 14.882 | 29.394 | 1.00 | 0.00 | 0.374 | 12.77 | 4.00 |
| ATOM | 3217 | HD1 | ARG | 303G | −14.360 | 14.823 | 29.701 | 1.00 | 0.00 | 0.053 | 0.00 | 0.00 |
| ATOM | 3218 | HD2 | ARG | 303G | −13.124 | 14.073 | 28.689 | 1.00 | 0.00 | 0.053 | 0.00 | 0.00 |
| ATOM | 3219 | NE | ARG | 303G | −13.119 | 16.146 | 28.697 | 1.00 | 0.00 | −0.819 | 9.00 | −24.67 |
| ATOM | 3220 | HE | ARG | 303G | −12.304 | 16.718 | 28.960 | 1.00 | 0.00 | 0.407 | 0.00 | 0.00 |
| ATOM | 3221 | CZ | ARG | 303G | −13.925 | 16.611 | 27.740 | 1.00 | 0.00 | 0.796 | 6.95 | 4.00 |
| ATOM | 3222 | NH1 | ARG | 303G | −14.991 | 15.911 | 27.356 | 1.00 | 0.00 | −0.746 | 9.00 | −24.67 |
| ATOM | 3223 | HH1 | ARG | 303G | −15.608 | 16.276 | 26.617 | 1.00 | 0.00 | 0.407 | 0.00 | 0.00 |
| ATOM | 3224 | HH1 | ARG | 303G | −15.198 | 15.004 | 27.798 | 1.00 | 0.00 | 0.407 | 0.00 | 0.00 |
| ATOM | 3225 | NH2 | ARG | 303G | −13.678 | 17.791 | 27.190 | 1.00 | 0.00 | −0.746 | 9.00 | −24.67 |
| ATOM | 3226 | HH2 | ARG | 303G | −14.296 | 18.154 | 26.451 | 1.00 | 0.00 | 0.407 | 0.00 | 0.00 |
| ATOM | 3227 | HH2 | ARG | 303G | −12.867 | 18.344 | 27.501 | 1.00 | 0.00 | 0.407 | 0.00 | 0.00 |
| ATOM | 3228 | C | ARG | 303G | −8.567 | 14.228 | 30.984 | 1.00 | 0.00 | 0.396 | 9.82 | 4.00 |
| ATOM | 3229 | O | ARG | 303G | −7.683 | 15.046 | 30.702 | 1.00 | 0.00 | −0.396 | 8.17 | −17.40 |
| ATOM | 3230 | N | ALA | 304 | −8.382 | 12.917 | 30.969 | 1.00 | 0.00 | −0.650 | 9.00 | −17.40 |
| ATOM | 3231 | HN | ALA | 304 | −9.177 | 12.300 | 31.188 | 1.00 | 0.00 | 0.440 | 0.00 | 0.00 |
| ATOM | 3232 | CA | ALA | 304 | −7.094 | 12.317 | 30.654 | 1.00 | 0.00 | 0.158 | 9.40 | 4.00 |
| ATOM | 3233 | HA | ALA | 304 | −6.379 | 13.119 | 30.470 | 1.00 | 0.00 | 0.053 | 0.00 | 0.00 |
| ATOM | 3234 | CB | ALA | 304 | −6.607 | 11.510 | 31.864 | 1.00 | 0.00 | −0.159 | 16.15 | 4.00 |
| ATOM | 3235 | HB1 | ALA | 304 | −5.642 | 11.057 | 31.635 | 1.00 | 0.00 | 0.053 | 0.00 | 0.00 |
| ATOM | 3236 | HB2 | ALA | 304 | −6.502 | 12.171 | 32.724 | 1.00 | 0.00 | 0.053 | 0.00 | 0.00 |
| ATOM | 3237 | HB3 | ALA | 304 | −7.329 | 10.727 | 32.094 | 1.00 | 0.00 | 0.053 | 0.00 | 0.00 |
| ATOM | 3238 | C | ALA | 304 | −7.100 | 11.419 | 29.430 | 1.00 | 0.00 | 0.396 | 9.82 | 4.00 |
| ATOM | 3239 | O | ALA | 304 | −8.152 | 11.084 | 28.868 | 1.00 | 0.00 | −0.396 | 8.17 | −17.40 |
| ATOM | 3240 | N | CYS | 305 | −5.902 | 11.032 | 29.009 | 1.00 | 0.00 | −0.650 | 9.00 | −17.40 |
| ATOM | 3241 | HN | CYS | 305 | −5.058 | 11.395 | 29.475 | 1.00 | 0.00 | 0.440 | 0.00 | 0.00 |
| ATOM | 3242 | CA | CYS | 305 | −5.762 | 10.103 | 27.900 | 1.00 | 0.00 | 0.158 | 9.40 | 4.00 |
| ATOM | 3243 | HA | CYS | 305 | −6.774 | 9.824 | 27.606 | 1.00 | 0.00 | 0.053 | 0.00 | 0.00 |
| ATOM | 3244 | C | CYS | 305 | −4.968 | 8.927 | 28.431 | 1.00 | 0.00 | 0.396 | 9.82 | 4.00 |
| ATOM | 3245 | O | CYS | 305 | −4.263 | 9.031 | 29.428 | 1.00 | 0.00 | −0.396 | 8.17 | −17.40 |
| ATOM | 3246 | CB | CYS | 305 | −4.917 | 10.648 | 26.734 | 1.00 | 0.00 | −0.041 | 12.77 | 4.00 |
| ATOM | 3247 | HB1 | CYS | 305 | −4.790 | 9.819 | 26.037 | 1.00 | 0.00 | 0.053 | 0.00 | 0.00 |
| ATOM | 3248 | HB2 | CYS | 305 | −3.972 | 10.975 | 27.169 | 1.00 | 0.00 | 0.053 | 0.00 | 0.00 |
| ATOM | 3249 | SG | CYS | 305 | −5.457 | 12.044 | 25.699 | 1.00 | 0.00 | −0.065 | 19.93 | −6.40 |
| ATOM | 3250 | N | VAL | 306 | −5.073 | 7.811 | 27.737 | 1.00 | 0.00 | −0.650 | 9.00 | −17.40 |
| ATOM | 3251 | HN | VAL | 306 | −5.720 | 7.764 | 26.937 | 1.00 | 0.00 | 0.440 | 0.00 | 0.00 |
| ATOM | 3252 | CA | VAL | 306 | −4.283 | 6.651 | 28.092 | 1.00 | 0.00 | 0.158 | 9.40 | 4.00 |
| ATOM | 3253 | HA | VAL | 306 | −4.012 | 6.659 | 29.148 | 1.00 | 0.00 | 0.053 | 0.00 | 0.00 |
| ATOM | 3254 | CB | VAL | 306 | −4.997 | 5.328 | 27.712 | 1.00 | 0.00 | −0.053 | 9.40 | 4.00 |
| ATOM | 3255 | HB | VAL | 306 | −5.387 | 5.392 | 26.696 | 1.00 | 0.00 | 0.053 | 0.00 | 0.00 |
| ATOM | 3256 | CG1 | VAL | 306 | −4.014 | 4.162 | 27.791 | 1.00 | 0.00 | −0.159 | 16.15 | 4.00 |
| ATOM | 3257 | HG1 | VAL | 306 | −4.524 | 3.237 | 27.522 | 1.00 | 0.00 | 0.053 | 0.00 | 0.00 |
| ATOM | 3258 | HG1 | VAL | 306 | −3.188 | 4.334 | 27.100 | 1.00 | 0.00 | 0.053 | 0.00 | 0.00 |
| ATOM | 3259 | HG1 | VAL | 306 | −3.626 | 4.080 | 28.806 | 1.00 | 0.00 | 0.053 | 0.00 | 0.00 |
| ATOM | 3260 | CG2 | VAL | 306 | −6.159 | 5.075 | 28.671 | 1.00 | 0.00 | −0.159 | 16.15 | 4.00 |
| ATOM | 3261 | HG2 | VAL | 306 | −6.658 | 4.144 | 28.400 | 1.00 | 0.00 | 0.053 | 0.00 | 0.00 |
| ATOM | 3262 | HG2 | VAL | 306 | −5.779 | 5.000 | 29.690 | 1.00 | 0.00 | 0.053 | 0.00 | 0.00 |
| ATOM | 3263 | HG2 | VAL | 306 | −6.869 | 5.899 | 28.607 | 1.00 | 0.00 | 0.053 | 0.00 | 0.00 |
| ATOM | 3264 | C | VAL | 306 | −3.082 | 6.816 | 27.184 | 1.00 | 0.00 | 0.396 | 9.82 | 4.00 |
| ATOM | 3265 | O | VAL | 306 | −3.229 | 7.262 | 26.042 | 1.00 | 0.00 | −0.396 | 8.17 | −17.40 |
| ATOM | 3266 | N | ARG | 307G | −1.897 | 6.515 | 27.698 | 1.00 | 0.00 | −0.650 | 9.00 | −17.40 |
| ATOM | 3267 | HN | ARG | 307G | −1.836 | 6.239 | 28.688 | 1.00 | 0.00 | 0.440 | 0.00 | 0.00 |
| ATOM | 3268 | CA | ARG | 307G | −0.673 | 6.563 | 26.897 | 1.00 | 0.00 | 0.158 | 9.40 | 4.00 |
| ATOM | 3269 | HA | ARG | 307G | −0.900 | 6.506 | 25.832 | 1.00 | 0.00 | 0.053 | 0.00 | 0.00 |
| ATOM | 3270 | CB | ARG | 307G | −0.096 | 7.892 | 27.089 | 1.00 | 0.00 | −0.106 | 12.77 | 4.00 |
| ATOM | 3271 | HB1 | ARG | 307G | −0.596 | 8.705 | 26.873 | 1.00 | 0.00 | 0.053 | 0.00 | 0.00 |
| ATOM | 3272 | HB2 | ARG | 307G | 0.934 | 7.889 | 26.392 | 1.00 | 0.00 | 0.053 | 0.00 | 0.00 |
| ATOM | 3273 | CG | ARG | 307G | 0.682 | 8.175 | 28.473 | 1.00 | 0.00 | −0.106 | 12.77 | 4.00 |
| ATOM | 3274 | HG1 | ARG | 307G | 0.019 | 7.796 | 29.252 | 1.00 | 0.00 | 0.053 | 0.00 | 0.00 |
| ATOM | 3275 | HG2 | ARG | 307G | 0.811 | 9.247 | 28.620 | 1.00 | 0.00 | 0.053 | 0.00 | 0.00 |
| ATOM | 3276 | CD | ARG | 307G | 2.041 | 7.500 | 28.625 | 1.00 | 0.00 | 0.374 | 12.77 | 4.00 |
| ATOM | 3277 | HD1 | ARG | 307G | 2.736 | 7.841 | 27.858 | 1.00 | 0.00 | 0.053 | 0.00 | 0.00 |
| ATOM | 3278 | HD2 | ARG | 307G | 1.952 | 6.417 | 28.537 | 1.00 | 0.00 | 0.053 | 0.00 | 0.00 |
| ATOM | 3279 | NE | ARG | 307G | 2.635 | 7.798 | 29.934 | 1.00 | 0.00 | −0.819 | 9.00 | −24.67 |
| ATOM | 3280 | HE | ARG | 307G | 2.131 | 8.436 | 30.566 | 1.00 | 0.00 | 0.407 | 0.00 | 0.00 |
| ATOM | 3281 | CZ | ARG | 307G | 3.788 | 7.286 | 30.342 | 1.00 | 0.00 | 0.796 | 6.95 | 4.00 |
| ATOM | 3282 | NH1 | ARG | 307G | 4.463 | 6.459 | 29.540 | 1.00 | 0.00 | −0.746 | 9.00 | −24.67 |
| ATOM | 3283 | HH1 | ARG | 307G | 5.359 | 6.057 | 29.850 | 1.00 | 0.00 | 0.407 | 0.00 | 0.00 |
| ATOM | 3284 | HH1 | ARG | 307G | 4.087 | 6.222 | 28.610 | 1.00 | 0.00 | 0.407 | 0.00 | 0.00 |
| ATOM | 3285 | NH2 | ARG | 307G | 4.250 | 7.583 | 31.545 | 1.00 | 0.00 | −0.746 | 9.00 | −24.67 |
| ATOM | 3286 | HH2 | ARG | 307G | 5.145 | 7.186 | 31.865 | 1.00 | 0.00 | 0.407 | 0.00 | 0.00 |
| ATOM | 3287 | HH2 | ARG | 307G | 3.714 | 8.210 | 32.162 | 1.00 | 0.00 | 0.407 | 0.00 | 0.00 |
| ATOM | 3288 | C | ARG | 307G | 0.120 | 5.332 | 27.378 | 1.00 | 0.00 | 0.396 | 9.82 | 4.00 |
| ATOM | 3289 | O | ARG | 307G | −0.068 | 4.868 | 28.503 | 1.00 | 0.00 | −0.396 | 8.17 | −17.40 |
| ATOM | 3290 | N | PRO | 308 | 0.998 | 4.784 | 26.525 | 1.00 | 0.00 | −0.422 | 9.00 | −17.40 |
| ATOM | 3291 | CD | PRO | 308 | 1.201 | 5.151 | 25.106 | 1.00 | 0.00 | 0.105 | 12.77 | 4.00 |
| ATOM | 3292 | HD1 | PRO | 308 | 1.688 | 6.121 | 25.016 | 1.00 | 0.00 | 0.053 | 0.00 | 0.00 |
| ATOM | 3293 | HD2 | PRO | 308 | 0.251 | 5.206 | 24.573 | 1.00 | 0.00 | 0.053 | 0.00 | 0.00 |
| ATOM | 3294 | CA | PRO | 308 | 1.785 | 3.601 | 26.880 | 1.00 | 0.00 | 0.158 | 9.40 | 4.00 |
| ATOM | 3295 | HA | PRO | 308 | 1.252 | 3.063 | 27.664 | 1.00 | 0.00 | 0.053 | 0.00 | 0.00 |
| ATOM | 3296 | CB | PRO | 308 | 1.851 | 2.852 | 25.563 | 1.00 | 0.00 | −0.106 | 12.77 | 4.00 |
| ATOM | 3297 | HB1 | PRO | 308 | 0.922 | 2.318 | 25.359 | 1.00 | 0.00 | 0.053 | 0.00 | 0.00 |
| ATOM | 3298 | HB2 | PRO | 308 | 2.655 | 2.116 | 25.560 | 1.00 | 0.00 | 0.053 | 0.00 | 0.00 |
| ATOM | 3299 | CG | PRO | 308 | 2.099 | 3.997 | 24.578 | 1.00 | 0.00 | −0.106 | 12.77 | 4.00 |
| ATOM | 3300 | HG1 | PRO | 308 | 1.787 | 3.590 | 23.615 | 1.00 | 0.00 | 0.053 | 0.00 | 0.00 |
| ATOM | 3301 | HG2 | PRO | 308 | 3.169 | 4.188 | 24.658 | 1.00 | 0.00 | 0.053 | 0.00 | 0.00 |
| ATOM | 3302 | C | PRO | 308 | 3.181 | 3.887 | 27.395 | 1.00 | 0.00 | 0.396 | 9.82 | 4.00 |
| ATOM | 3303 | O | PRO | 308 | 3.708 | 4.975 | 27.188 | 1.00 | 0.00 | −0.396 | 8.17 | −17.40 |
| ATOM | 3304 | N | GLU | 309 | 3.780 | 2.881 | 28.031 | 1.00 | 0.00 | −0.650 | 9.00 | −17.40 |
| ATOM | 3305 | HN | GLU | 309 | 3.253 | 2.010 | 28.189 | 1.00 | 0.00 | 0.440 | 0.00 | 0.00 |
| ATOM | 3306 | CA | GLU | 309 | 5.156 | 2.968 | 28.511 | 1.00 | 0.00 | 0.158 | 9.40 | 4.00 |
| ATOM | 3307 | HA | GLU | 309 | 5.231 | 3.849 | 29.147 | 1.00 | 0.00 | 0.053 | 0.00 | 0.00 |
| ATOM | 3308 | CB | GLU | 309 | 5.505 | 1.689 | 29.285 | 1.00 | 0.00 | −0.106 | 12.77 | 4.00 |
| ATOM | 3309 | HB1 | GLU | 309 | 5.222 | 0.838 | 28.664 | 1.00 | 0.00 | 0.053 | 0.00 | 0.00 |
| ATOM | 3310 | HB2 | GLU | 309 | 4.940 | 1.701 | 30.217 | 1.00 | 0.00 | 0.053 | 0.00 | 0.00 |
| ATOM | 3311 | CG | GLU | 309 | 6.992 | 1.488 | 29.667 | 1.00 | 0.00 | −0.106 | 12.77 | 4.00 |
| ATOM | 3312 | HG1 | GLU | 309 | 7.682 | 1.739 | 28.862 | 1.00 | 0.00 | 0.053 | 0.00 | 0.00 |
| ATOM | 3313 | HG2 | GLU | 309 | 7.232 | 0.461 | 29.942 | 1.00 | 0.00 | 0.053 | 0.00 | 0.00 |
| ATOM | 3314 | CD | GLU | 309 | 7.427 | 2.330 | 30.844 | 1.00 | 0.00 | 0.399 | 9.82 | 4.00 |
| ATOM | 3315 | OE1 | GLU | 309 | 6.554 | 2.982 | 31.453 | 1.00 | 0.00 | −0.396 | 8.17 | −18.95 |
| ATOM | 3316 | OE2 | GLU | 309 | 8.642 | 2.334 | 31.166 | 1.00 | 0.00 | −0.427 | 8.17 | −18.95 |
| ATOM | 3317 | HE2 | GLU | 309 | 8.778 | 2.944 | 31.984 | 1.00 | 0.00 | 0.424 | 0.00 | 0.00 |
| ATOM | 3318 | C | GLU | 309 | 6.074 | 3.090 | 27.285 | 1.00 | 0.00 | 0.396 | 9.82 | 4.00 |
| ATOM | 3319 | O | GLU | 309 | 7.065 | 3.829 | 27.314 | 1.00 | 0.00 | −0.396 | 8.17 | −17.40 |
| ATOM | 3320 | N | ILE | 310 | 5.748 | 2.350 | 26.219 | 1.00 | 0.00 | −0.650 | 9.00 | −17.40 |
| ATOM | 3321 | HN | ILE | 310 | 4.904 | 1.760 | 26.267 | 1.00 | 0.00 | 0.440 | 0.00 | 0.00 |
| ATOM | 3322 | CA | ILE | 310 | 6.544 | 2.343 | 24.980 | 1.00 | 0.00 | 0.158 | 9.40 | 4.00 |
| ATOM | 3323 | HA | ILE | 310 | 7.358 | 3.052 | 25.126 | 1.00 | 0.00 | 0.053 | 0.00 | 0.00 |
| ATOM | 3324 | CB | ILE | 310 | 7.118 | 0.916 | 24.698 | 1.00 | 0.00 | −0.053 | 9.40 | 4.00 |
| ATOM | 3325 | HB | ILE | 310 | 6.313 | 0.188 | 24.590 | 1.00 | 0.00 | 0.053 | 0.00 | 0.00 |
| ATOM | 3326 | CG2 | ILE | 310 | 7.943 | 0.905 | 23.409 | 1.00 | 0.00 | −0.159 | 16.15 | 4.00 |
| ATOM | 3327 | HG2 | ILE | 310 | 8.331 | −0.098 | 23.234 | 1.00 | 0.00 | 0.053 | 0.00 | 0.00 |
| ATOM | 3328 | HG2 | ILE | 310 | 7.312 | 1.200 | 22.570 | 1.00 | 0.00 | 0.053 | 0.00 | 0.00 |
| ATOM | 3329 | HG2 | ILE | 310 | 8.773 | 1.604 | 23.502 | 1.00 | 0.00 | 0.053 | 0.00 | 0.00 |
| ATOM | 3330 | CG1 | ILE | 310 | 7.970 | 0.458 | 25.883 | 1.00 | 0.00 | −0.106 | 12.77 | 4.00 |
| ATOM | 3331 | HG1 | ILE | 310 | 7.331 | 0.402 | 26.764 | 1.00 | 0.00 | 0.053 | 0.00 | 0.00 |
| ATOM | 3332 | HG1 | ILE | 310 | 8.768 | 1.184 | 26.034 | 1.00 | 0.00 | 0.053 | 0.00 | 0.00 |
| ATOM | 3333 | CD1 | ILE | 310 | 8.632 | −0.944 | 25.683 | 1.00 | 0.00 | −0.159 | 16.15 | 4.00 |
| ATOM | 3334 | HD1 | ILE | 310 | 9.219 | −1.198 | 26.565 | 1.00 | 0.00 | 0.053 | 0.00 | 0.00 |
| ATOM | 3335 | HD1 | ILE | 310 | 7.856 | −1.695 | 25.534 | 1.00 | 0.00 | 0.053 | 0.00 | 0.00 |
| ATOM | 3336 | HD1 | ILE | 310 | 9.283 | −0.917 | 24.809 | 1.00 | 0.00 | 0.053 | 0.00 | 0.00 |
| ATOM | 3337 | C | ILE | 310 | 5.604 | 2.771 | 23.849 | 1.00 | 0.00 | 0.396 | 9.82 | 4.00 |
| ATOM | 3338 | O | ILE | 310 | 4.538 | 2.167 | 23.660 | 1.00 | 0.00 | −0.396 | 8.17 | −17.40 |
| ATOM | 3339 | N | SER | 311 | 5.997 | 3.802 | 23.102 | 1.00 | 0.00 | −0.650 | 9.00 | −17.40 |
| ATOM | 3340 | HN | SER | 311 | 6.923 | 4.219 | 23.272 | 1.00 | 0.00 | 0.440 | 0.00 | 0.00 |
| ATOM | 3341 | CA | SER | 311 | 5.145 | 4.360 | 22.041 | 1.00 | 0.00 | 0.158 | 9.40 | 4.00 |
| ATOM | 3342 | HA | SER | 311 | 4.230 | 4.729 | 22.505 | 1.00 | 0.00 | 0.053 | 0.00 | 0.00 |
| ATOM | 3343 | CB | SER | 311 | 5.818 | 5.584 | 21.403 | 1.00 | 0.00 | 0.007 | 12.77 | 4.00 |
| ATOM | 3344 | HB1 | SER | 311 | 5.154 | 6.082 | 20.696 | 1.00 | 0.00 | 0.053 | 0.00 | 0.00 |
| ATOM | 3345 | HB2 | SER | 311 | 6.721 | 5.306 | 20.859 | 1.00 | 0.00 | 0.053 | 0.00 | 0.00 |
| ATOM | 3346 | OG | SER | 311 | 6.190 | 6.540 | 22.378 | 1.00 | 0.00 | −0.537 | 11.04 | −17.40 |
| ATOM | 3347 | HG | SER | 311 | 6.936 | 7.142 | 22.002 | 1.00 | 0.00 | 0.424 | 0.00 | 0.00 |
| ATOM | 3348 | C | SER | 311 | 4.727 | 3.384 | 20.937 | 1.00 | 0.00 | 0.396 | 9.82 | 4.00 |
| ATOM | 3349 | O | SER | 311 | 5.477 | 2.475 | 20.563 | 1.00 | 0.00 | −0.396 | 8.17 | −17.40 |
| ATOM | 3350 | N | ARG | 312G | 3.514 | 3.578 | 20.421 | 1.00 | 0.00 | −0.650 | 9.00 | −17.40 |
| ATOM | 3351 | HN | ARG | 312G | 2.918 | 4.333 | 20.787 | 1.00 | 0.00 | 0.440 | 0.00 | 0.00 |
| ATOM | 3352 | CA | ARG | 312G | 3.024 | 2.731 | 19.344 | 1.00 | 0.00 | 0.158 | 9.40 | 4.00 |
| ATOM | 3353 | HA | ARG | 312G | 3.596 | 1.805 | 19.286 | 1.00 | 0.00 | 0.053 | 0.00 | 0.00 |
| ATOM | 3354 | CB | ARG | 312G | 1.594 | 2.269 | 19.623 | 1.00 | 0.00 | −0.106 | 12.77 | 4.00 |
| ATOM | 3355 | HB1 | ARG | 312G | 0.979 | 2.341 | 18.725 | 1.00 | 0.00 | 0.053 | 0.00 | 0.00 |
| ATOM | 3356 | HB2 | ARG | 312G | 1.131 | 2.880 | 20.397 | 1.00 | 0.00 | 0.053 | 0.00 | 0.00 |
| ATOM | 3357 | CG | ARG | 312G | 1.572 | 0.796 | 20.100 | 1.00 | 0.00 | −0.106 | 12.77 | 4.00 |
| ATOM | 3358 | HG1 | ARG | 312G | 2.014 | 0.155 | 19.337 | 1.00 | 0.00 | 0.053 | 0.00 | 0.00 |
| ATOM | 3359 | HG2 | ARG | 312G | 0.543 | 0.482 | 20.279 | 1.00 | 0.00 | 0.053 | 0.00 | 0.00 |
| ATOM | 3360 | CD | ARG | 312G | 2.380 | 0.660 | 21.408 | 1.00 | 0.00 | 0.374 | 12.77 | 4.00 |
| ATOM | 3361 | HD1 | ARG | 312G | 1.917 | 1.200 | 22.234 | 1.00 | 0.00 | 0.053 | 0.00 | 0.00 |
| ATOM | 3362 | HD2 | ARG | 312G | 3.393 | 1.049 | 21.307 | 1.00 | 0.00 | 0.053 | 0.00 | 0.00 |
| ATOM | 3363 | NE | ARG | 312G | 2.506 | −0.732 | 21.835 | 1.00 | 0.00 | −0.819 | 9.00 | −24.67 |
| ATOM | 3364 | HE | ARG | 312G | 2.293 | −1.467 | 21.145 | 1.00 | 0.00 | 0.407 | 0.00 | 0.00 |
| ATOM | 3365 | CZ | ARG | 312G | 2.878 | −1.115 | 23.059 | 1.00 | 0.00 | 0.796 | 6.95 | 4.00 |
| ATOM | 3366 | NH1 | ARG | 312G | 3.163 | −0.211 | 23.975 | 1.00 | 0.00 | −0.746 | 9.00 | −24.67 |
| ATOM | 3367 | HH1 | ARG | 312G | 3.449 | −0.507 | 24.919 | 1.00 | 0.00 | 0.407 | 0.00 | 0.00 |
| ATOM | 3368 | HH1 | ARG | 312G | 3.098 | 0.791 | 23.746 | 1.00 | 0.00 | 0.407 | 0.00 | 0.00 |
| ATOM | 3369 | NH2 | ARG | 312G | 2.945 | −2.405 | 23.371 | 1.00 | 0.00 | −0.746 | 9.00 | −24.67 |
| ATOM | 3370 | HH2 | ARG | 312G | 3.232 | −2.691 | 24.317 | 1.00 | 0.00 | 0.407 | 0.00 | 0.00 |
| ATOM | 3371 | HH2 | ARG | 312G | 2.708 | −3.118 | 22.666 | 1.00 | 0.00 | 0.407 | 0.00 | 0.00 |
| ATOM | 3372 | C | ARG | 312G | 3.138 | 3.437 | 18.006 | 1.00 | 0.00 | 0.396 | 9.82 | 4.00 |
| ATOM | 3373 | O | ARG | 312G | 2.646 | 2.960 | 16.984 | 1.00 | 0.00 | −0.396 | 8.17 | −17.40 |
| ATOM | 3374 | N | THR | 313 | 3.791 | 4.596 | 18.035 | 1.00 | 0.00 | −0.650 | 9.00 | −17.40 |
| ATOM | 3375 | HN | THR | 313 | 4.113 | 4.961 | 18.942 | 1.00 | 0.00 | 0.440 | 0.00 | 0.00 |
| ATOM | 3376 | CA | THR | 313 | 4.068 | 5.370 | 16.823 | 1.00 | 0.00 | 0.158 | 9.40 | 4.00 |
| ATOM | 3377 | HA | THR | 313 | 4.095 | 4.668 | 15.989 | 1.00 | 0.00 | 0.053 | 0.00 | 0.00 |
| ATOM | 3378 | CB | THR | 313 | 3.042 | 6.491 | 16.534 | 1.00 | 0.00 | 0.060 | 9.40 | 4.00 |
| ATOM | 3379 | HB | THR | 313 | 3.398 | 7.138 | 15.733 | 1.00 | 0.00 | 0.053 | 0.00 | 0.00 |
| ATOM | 3380 | OG1 | THR | 313 | 2.888 | 7.307 | 17.698 | 1.00 | 0.00 | −0.537 | 11.04 | −17.40 |
| ATOM | 3381 | HG1 | THR | 313 | 3.405 | 8.188 | 17.570 | 1.00 | 0.00 | 0.424 | 0.00 | 0.00 |
| ATOM | 3382 | CG2 | THR | 313 | 1.703 | 5.895 | 16.111 | 1.00 | 0.00 | −0.159 | 16.15 | 4.00 |
| ATOM | 3383 | HG2 | THR | 313 | 0.993 | 6.698 | 15.912 | 1.00 | 0.00 | 0.053 | 0.00 | 0.00 |
| ATOM | 3384 | HG2 | THR | 313 | 1.838 | 5.299 | 15.208 | 1.00 | 0.00 | 0.053 | 0.00 | 0.00 |
| ATOM | 3385 | HG2 | THR | 313 | 1.318 | 5.261 | 16.910 | 1.00 | 0.00 | 0.053 | 0.00 | 0.00 |
| ATOM | 3386 | C | THR | 313 | 5.413 | 6.056 | 17.021 | 1.00 | 0.00 | 0.396 | 9.82 | 4.00 |
| ATOM | 3387 | O | THR | 313 | 5.884 | 6.212 | 18.151 | 1.00 | 0.00 | −0.396 | 8.17 | −17.40 |
| ATOM | 3388 | N | MET | 314 | 6.019 | 6.462 | 15.914 | 1.00 | 0.00 | −0.650 | 9.00 | −17.40 |
| ATOM | 3389 | HN | MET | 314 | 5.572 | 6.270 | 15.005 | 1.00 | 0.00 | 0.440 | 0.00 | 0.00 |
| ATOM | 3390 | CA | MET | 314 | 7.289 | 7.167 | 15.935 | 1.00 | 0.00 | 0.158 | 9.40 | 4.00 |
| ATOM | 3391 | HA | MET | 314 | 7.438 | 7.681 | 16.884 | 1.00 | 0.00 | 0.053 | 0.00 | 0.00 |
| ATOM | 3392 | CB | MET | 314 | 8.464 | 6.215 | 15.657 | 1.00 | 0.00 | −0.106 | 12.77 | 4.00 |
| ATOM | 3393 | HB1 | MET | 314 | 8.506 | 5.368 | 16.342 | 1.00 | 0.00 | 0.053 | 0.00 | 0.00 |
| ATOM | 3394 | HB2 | MET | 314 | 9.438 | 6.696 | 15.734 | 1.00 | 0.00 | 0.053 | 0.00 | 0.00 |
| ATOM | 3395 | CG | MET | 314 | 8.428 | 5.598 | 14.256 | 1.00 | 0.00 | −0.041 | 12.77 | 4.00 |
| ATOM | 3396 | HG1 | MET | 314 | 8.216 | 6.391 | 13.539 | 1.00 | 0.00 | 0.053 | 0.00 | 0.00 |
| ATOM | 3397 | HG2 | MET | 314 | 7.642 | 4.842 | 14.236 | 1.00 | 0.00 | 0.053 | 0.00 | 0.00 |
| ATOM | 3398 | SD | MET | 314 | 9.960 | 4.803 | 13.750 | 1.00 | 0.00 | −0.130 | 16.39 | −6.40 |
| ATOM | 3399 | CE | MET | 314 | 10.989 | 6.220 | 13.391 | 1.00 | 0.00 | −0.094 | 16.15 | 4.00 |
| ATOM | 3400 | HE1 | MET | 314 | 11.972 | 5.882 | 13.064 | 1.00 | 0.00 | 0.053 | 0.00 | 0.00 |
| ATOM | 3401 | HE2 | MET | 314 | 10.528 | 6.813 | 12.601 | 1.00 | 0.00 | 0.053 | 0.00 | 0.00 |
| ATOM | 3402 | HE3 | MET | 314 | 11.094 | 6.829 | 14.288 | 1.00 | 0.00 | 0.053 | 0.00 | 0.00 |
| ATOM | 3403 | C | MET | 314 | 7.154 | 8.135 | 14.781 | 1.00 | 0.00 | 0.396 | 9.82 | 4.00 |
| ATOM | 3404 | O | MET | 314 | 6.330 | 7.927 | 13.891 | 1.00 | 0.00 | −0.396 | 8.17 | −17.40 |
| ATOM | 3405 | N | THR | 315 | 7.938 | 9.200 | 14.794 | 1.00 | 0.00 | −0.650 | 9.00 | −17.40 |
| ATOM | 3406 | HN | THR | 315 | 8.584 | 9.356 | 15.581 | 1.00 | 0.00 | 0.440 | 0.00 | 0.00 |
| ATOM | 3407 | CA | THR | 315 | 7.885 | 10.146 | 13.697 | 1.00 | 0.00 | 0.158 | 9.40 | 4.00 |
| ATOM | 3408 | HA | THR | 315 | 7.132 | 9.887 | 12.952 | 1.00 | 0.00 | 0.053 | 0.00 | 0.00 |
| ATOM | 3409 | CB | THR | 315 | 7.405 | 11.578 | 14.148 | 1.00 | 0.00 | 0.060 | 9.40 | 4.00 |
| ATOM | 3410 | HB | THR | 315 | 6.467 | 11.541 | 14.702 | 1.00 | 0.00 | 0.053 | 0.00 | 0.00 |
| ATOM | 3411 | OG1 | THR | 315 | 7.200 | 12.401 | 12.987 | 1.00 | 0.00 | −0.537 | 11.04 | −17.40 |
| ATOM | 3412 | HG1 | THR | 315 | 7.706 | 13.290 | 13.102 | 1.00 | 0.00 | 0.424 | 0.00 | 0.00 |
| ATOM | 3413 | CG2 | THR | 315 | 8.420 | 12.265 | 15.051 | 1.00 | 0.00 | −0.159 | 16.15 | 4.00 |
| ATOM | 3414 | HG2 | THR | 315 | 8.046 | 13.248 | 15.336 | 1.00 | 0.00 | 0.053 | 0.00 | 0.00 |
| ATOM | 3415 | HG2 | THR | 315 | 8.577 | 11.662 | 15.946 | 1.00 | 0.00 | 0.053 | 0.00 | 0.00 |
| ATOM | 3416 | HG2 | THR | 315 | 9.364 | 12.376 | 14.518 | 1.00 | 0.00 | 0.053 | 0.00 | 0.00 |
| ATOM | 3417 | C | THR | 315 | 9.205 | 10.248 | 12.941 | 1.00 | 0.00 | 0.396 | 9.82 | 4.00 |
| ATOM | 3418 | O | THR | 315 | 10.294 | 10.206 | 13.522 | 1.00 | 0.00 | −0.396 | 8.17 | −17.40 |
| ATOM | 3419 | N | PHE | 316 | 9.081 | 10.361 | 11.624 | 1.00 | 0.00 | −0.650 | 9.00 | −17.40 |
| ATOM | 3420 | HN | PHE | 316 | 8.137 | 10.348 | 11.210 | 1.00 | 0.00 | 0.440 | 0.00 | 0.00 |
| ATOM | 3421 | CA | PHE | 316 | 10.233 | 10.502 | 10.743 | 1.00 | 0.00 | 0.158 | 9.40 | 4.00 |
| ATOM | 3422 | HA | PHE | 316 | 11.143 | 10.512 | 11.342 | 1.00 | 0.00 | 0.053 | 0.00 | 0.00 |
| ATOM | 3423 | CB | PHE | 316 | 10.324 | 9.296 | 9.806 | 1.00 | 0.00 | −0.106 | 12.77 | 4.00 |
| ATOM | 3424 | HB1 | PHE | 316 | 10.536 | 8.411 | 10.406 | 1.00 | 0.00 | 0.053 | 0.00 | 0.00 |
| ATOM | 3425 | HB2 | PHE | 316 | 11.127 | 9.476 | 9.092 | 1.00 | 0.00 | 0.053 | 0.00 | 0.00 |
| ATOM | 3426 | CG | PHE | 316 | 9.062 | 9.027 | 9.020 | 1.00 | 0.00 | 0.000 | 7.26 | 0.60 |
| ATOM | 3427 | CD1 | PHE | 316 | 8.168 | 8.047 | 9.427 | 1.00 | 0.00 | −0.127 | 10.80 | 0.60 |
| ATOM | 3428 | HD1 | PHE | 316 | 8.375 | 7.469 | 10.328 | 1.00 | 0.00 | 0.127 | 0.00 | 0.00 |
| ATOM | 3429 | CD2 | PHE | 316 | 8.781 | 9.757 | 7.865 | 1.00 | 0.00 | −0.127 | 10.80 | 0.60 |
| ATOM | 3430 | HD2 | PHE | 316 | 9.475 | 10.529 | 7.535 | 1.00 | 0.00 | 0.127 | 0.00 | 0.00 |
| ATOM | 3431 | CE1 | PHE | 316 | 7.007 | 7.792 | 8.696 | 1.00 | 0.00 | −0.127 | 10.80 | 0.60 |
| ATOM | 3432 | HE1 | PHE | 316 | 6.312 | 7.019 | 9.026 | 1.00 | 0.00 | 0.127 | 0.00 | 0.00 |
| ATOM | 3433 | CE2 | PHE | 316 | 7.622 | 9.511 | 7.125 | 1.00 | 0.00 | −0.127 | 10.80 | 0.60 |
| ATOM | 3434 | HE2 | PHE | 316 | 7.413 | 10.088 | 6.224 | 1.00 | 0.00 | 0.127 | 0.00 | 0.00 |
| ATOM | 3435 | CZ | PHE | 316 | 6.737 | 8.527 | 7.543 | 1.00 | 0.00 | −0.127 | 10.80 | 0.60 |
| ATOM | 3436 | HZ | PHE | 316 | 5.831 | 8.329 | 6.970 | 1.00 | 0.00 | 0.127 | 0.00 | 0.00 |
| ATOM | 3437 | C | PHE | 316 | 10.111 | 11.828 | 9.955 | 1.00 | 0.00 | 0.396 | 9.82 | 4.00 |
| ATOM | 3438 | O | PHE | 316 | 10.933 | 12.134 | 9.093 | 1.00 | 0.00 | −0.396 | 8.17 | −17.40 |
| ATOM | 3439 | N | GLY | 317 | 9.095 | 12.623 | 10.296 | 1.00 | 0.00 | −0.650 | 9.00 | −17.40 |
| ATOM | 3440 | HN | GLY | 317 | 8.455 | 12.320 | 11.044 | 1.00 | 0.00 | 0.440 | 0.00 | 0.00 |
| ATOM | 3441 | CA | GLY | 317 | 8.861 | 13.910 | 9.641 | 1.00 | 0.00 | 0.105 | 9.40 | 4.00 |
| ATOM | 3442 | HA1 | GLY | 317 | 7.853 | 14.233 | 9.902 | 1.00 | 0.00 | 0.053 | 0.00 | 0.00 |
| ATOM | 3443 | HA2 | GLY | 317 | 8.961 | 13.755 | 8.566 | 1.00 | 0.00 | 0.053 | 0.00 | 0.00 |
| ATOM | 3444 | C | GLY | 317 | 9.796 | 15.061 | 10.005 | 1.00 | 0.00 | 0.396 | 9.82 | 4.00 |
| ATOM | 3445 | O | GLY | 317 | 9.382 | 16.088 | 10.594 | 1.00 | 0.00 | −0.396 | 8.17 | −17.40 |
| ATOM | 3446 | N | ARG | 318G | 11.062 | 14.887 | 9.623 | 1.00 | 0.00 | −0.650 | 9.00 | −17.40 |
| ATOM | 3447 | HN | ARG | 318G | 11.298 | 14.008 | 9.140 | 1.00 | 0.00 | 0.440 | 0.00 | 0.00 |
| ATOM | 3448 | CA | ARG | 318G | 12.138 | 15.853 | 9.839 | 1.00 | 0.00 | 0.158 | 9.40 | 4.00 |
| ATOM | 3449 | HA | ARG | 318G | 12.379 | 15.950 | 10.897 | 1.00 | 0.00 | 0.053 | 0.00 | 0.00 |
| ATOM | 3450 | CB | ARG | 318G | 13.382 | 15.375 | 9.089 | 1.00 | 0.00 | −0.106 | 12.77 | 4.00 |
| ATOM | 3451 | HB1 | ARG | 318G | 14.274 | 15.950 | 9.333 | 1.00 | 0.00 | 0.053 | 0.00 | 0.00 |
| ATOM | 3452 | HB2 | ARG | 318G | 13.277 | 15.438 | 8.006 | 1.00 | 0.00 | 0.053 | 0.00 | 0.00 |
| ATOM | 3453 | CG | ARG | 318G | 13.733 | 13.931 | 9.383 | 1.00 | 0.00 | −0.106 | 12.77 | 4.00 |
| ATOM | 3454 | HG1 | ARG | 318G | 12.928 | 13.255 | 9.092 | 1.00 | 0.00 | 0.053 | 0.00 | 0.00 |
| ATOM | 3455 | HG2 | ARG | 318G | 13.921 | 13.771 | 10.444 | 1.00 | 0.00 | 0.053 | 0.00 | 0.00 |
| ATOM | 3456 | CD | ARG | 318G | 14.989 | 13.492 | 8.629 | 1.00 | 0.00 | 0.374 | 12.77 | 4.00 |
| ATOM | 3457 | HD1 | ARG | 318G | 15.637 | 14.341 | 8.411 | 1.00 | 0.00 | 0.053 | 0.00 | 0.00 |
| ATOM | 3458 | HD2 | ARG | 318G | 14.736 | 13.018 | 7.680 | 1.00 | 0.00 | 0.053 | 0.00 | 0.00 |
| ATOM | 3459 | NE | ARG | 318G | 15.768 | 12.530 | 9.409 | 1.00 | 0.00 | −0.819 | 9.00 | −24.67 |
| ATOM | 3460 | HE | ARG | 318G | 15.871 | 11.573 | 9.041 | 1.00 | 0.00 | 0.407 | 0.00 | 0.00 |
| ATOM | 3461 | CZ | ARG | 318G | 16.349 | 12.827 | 10.567 | 1.00 | 0.00 | 0.796 | 6.95 | 4.00 |
| ATOM | 3462 | NH1 | ARG | 318G | 16.228 | 14.053 | 11.061 | 1.00 | 0.00 | −0.746 | 9.00 | −24.67 |
| ATOM | 3463 | HH1 | ARG | 318G | 16.674 | 14.293 | 11.957 | 1.00 | 0.00 | 0.407 | 0.00 | 0.00 |
| ATOM | 3464 | HH1 | ARG | 318G | 15.687 | 14.764 | 10.547 | 1.00 | 0.00 | 0.407 | 0.00 | 0.00 |
| ATOM | 3465 | NH2 | ARG | 318G | 17.049 | 11.912 | 11.228 | 1.00 | 0.00 | −0.746 | 9.00 | −24.67 |
| ATOM | 3466 | HH2 | ARG | 318G | 17.495 | 12.153 | 12.124 | 1.00 | 0.00 | 0.407 | 0.00 | 0.00 |
| ATOM | 3467 | HH2 | ARG | 318G | 17.145 | 10.961 | 10.844 | 1.00 | 0.00 | 0.407 | 0.00 | 0.00 |
| ATOM | 3468 | C | ARG | 318G | 11.754 | 17.241 | 9.327 | 1.00 | 0.00 | 0.396 | 9.82 | 4.00 |
| ATOM | 3469 | O | ARG | 318G | 11.783 | 18.238 | 10.066 | 1.00 | 0.00 | −0.396 | 8.17 | −17.40 |
| ATOM | 3470 | N | LYS | 319S | 11.404 | 17.279 | 8.045 | 1.00 | 0.00 | −0.650 | 9.00 | −17.40 |
| ATOM | 3471 | HN | LYS | 319S | 11.423 | 16.399 | 7.509 | 1.00 | 0.00 | 0.440 | 0.00 | 0.00 |
| ATOM | 3472 | CA | LYS | 319S | 10.993 | 18.499 | 7.353 | 1.00 | 0.00 | 0.158 | 9.40 | 4.00 |
| ATOM | 3473 | HA | LYS | 319S | 11.381 | 19.341 | 7.925 | 1.00 | 0.00 | 0.053 | 0.00 | 0.00 |
| ATOM | 3474 | CB | LYS | 319S | 11.571 | 18.496 | 5.924 | 1.00 | 0.00 | −0.106 | 12.77 | 4.00 |
| ATOM | 3475 | HB1 | LYS | 319S | 11.433 | 17.497 | 5.509 | 1.00 | 0.00 | 0.053 | 0.00 | 0.00 |
| ATOM | 3476 | HB2 | LYS | 319S | 12.629 | 18.749 | 5.989 | 1.00 | 0.00 | 0.053 | 0.00 | 0.00 |
| ATOM | 3477 | CG | LYS | 319S | 10.933 | 19.481 | 4.946 | 1.00 | 0.00 | −0.106 | 12.77 | 4.00 |
| ATOM | 3478 | HG1 | LYS | 319S | 9.843 | 19.454 | 4.977 | 1.00 | 0.00 | 0.053 | 0.00 | 0.00 |
| ATOM | 3479 | HG2 | LYS | 319S | 11.213 | 19.283 | 3.911 | 1.00 | 0.00 | 0.053 | 0.00 | 0.00 |
| ATOM | 3480 | CD | LYS | 319S | 11.331 | 20.919 | 5.226 | 1.00 | 0.00 | −0.106 | 12.77 | 4.00 |
| ATOM | 3481 | HD1 | LYS | 319S | 12.405 | 21.047 | 5.097 | 1.00 | 0.00 | 0.053 | 0.00 | 0.00 |
| ATOM | 3482 | HD2 | LYS | 319S | 11.069 | 21.193 | 6.248 | 1.00 | 0.00 | 0.053 | 0.00 | 0.00 |
| ATOM | 3483 | CE | LYS | 319S | 10.614 | 21.862 | 4.274 | 1.00 | 0.00 | 0.099 | 12.77 | 4.00 |
| ATOM | 3484 | HE1 | LYS | 319S | 10.882 | 21.636 | 3.241 | 1.00 | 0.00 | 0.053 | 0.00 | 0.00 |
| ATOM | 3485 | HE2 | LYS | 319S | 10.887 | 22.895 | 4.485 | 1.00 | 0.00 | 0.053 | 0.00 | 0.00 |
| ATOM | 3486 | NZ | LYS | 319S | 9.133 | 21.724 | 4.420 | 1.00 | 0.00 | −0.045 | 13.25 | −39.20 |
| ATOM | 3487 | HZ1 | LYS | 319S | 8.661 | 22.368 | 3.769 | 1.00 | 0.00 | 0.280 | 0.00 | 0.00 |
| ATOM | 3488 | HZ2 | LYS | 319S | 8.855 | 20.755 | 4.205 | 1.00 | 0.00 | 0.280 | 0.00 | 0.00 |
| ATOM | 3489 | HZ3 | LYS | 319S | 8.860 | 21.952 | 5.386 | 1.00 | 0.00 | 0.280 | 0.00 | 0.00 |
| ATOM | 3490 | C | LYS | 319S | 9.467 | 18.503 | 7.312 | 1.00 | 0.00 | 0.396 | 9.82 | 4.00 |
| ATOM | 3491 | O | LYS | 319S | 8.849 | 17.531 | 6.874 | 1.00 | 0.00 | −0.396 | 8.17 | −17.40 |
| ATOM | 3492 | N | GLY | 320 | 8.866 | 19.590 | 7.782 | 1.00 | 0.00 | −0.650 | 9.00 | −17.40 |
| ATOM | 3493 | HN | GLY | 320 | 9.434 | 20.366 | 8.149 | 1.00 | 0.00 | 0.440 | 0.00 | 0.00 |
| ATOM | 3494 | CA | GLY | 320 | 7.420 | 19.697 | 7.784 | 1.00 | 0.00 | 0.105 | 9.40 | 4.00 |
| ATOM | 3495 | HA1 | GLY | 320 | 6.973 | 19.122 | 8.595 | 1.00 | 0.00 | 0.053 | 0.00 | 0.00 |
| ATOM | 3496 | HA2 | GLY | 320 | 6.986 | 19.329 | 6.854 | 1.00 | 0.00 | 0.053 | 0.00 | 0.00 |
| ATOM | 3497 | C | GLY | 320 | 6.964 | 21.130 | 7.951 | 1.00 | 0.00 | 0.396 | 9.82 | 4.00 |
| ATOM | 3498 | O | GLY | 320 | 7.638 | 22.068 | 7.492 | 1.00 | 0.00 | −0.396 | 8.17 | −17.40 |
| ATOM | 3499 | N | VAL | 321 | 5.809 | 21.317 | 8.582 | 1.00 | 0.00 | −0.650 | 9.00 | −17.40 |
| ATOM | 3500 | HN | VAL | 321 | 5.263 | 20.505 | 8.905 | 1.00 | 0.00 | 0.440 | 0.00 | 0.00 |
| ATOM | 3501 | CA | VAL | 321 | 5.312 | 22.665 | 8.817 | 1.00 | 0.00 | 0.158 | 9.40 | 4.00 |
| ATOM | 3502 | HA | VAL | 321 | 5.337 | 23.172 | 7.852 | 1.00 | 0.00 | 0.053 | 0.00 | 0.00 |
| ATOM | 3503 | CB | VAL | 321 | 3.883 | 22.646 | 9.373 | 1.00 | 0.00 | −0.053 | 9.40 | 4.00 |
| ATOM | 3504 | HB | VAL | 321 | 3.812 | 21.963 | 10.220 | 1.00 | 0.00 | 0.053 | 0.00 | 0.00 |
| ATOM | 3505 | CG1 | VAL | 321 | 3.473 | 24.040 | 9.841 | 1.00 | 0.00 | −0.159 | 16.15 | 4.00 |
| ATOM | 3506 | HG1 | VAL | 321 | 2.456 | 24.007 | 10.232 | 1.00 | 0.00 | 0.053 | 0.00 | 0.00 |
| ATOM | 3507 | HG1 | VAL | 321 | 4.152 | 24.375 | 10.624 | 1.00 | 0.00 | 0.053 | 0.00 | 0.00 |
| ATOM | 3508 | HG1 | VAL | 321 | 3.516 | 24.733 | 9.001 | 1.00 | 0.00 | 0.053 | 0.00 | 0.00 |
| ATOM | 3509 | CG2 | VAL | 321 | 2.942 | 22.152 | 8.294 | 1.00 | 0.00 | −0.159 | 16.15 | 4.00 |
| ATOM | 3510 | HG2 | VAL | 321 | 1.922 | 22.134 | 8.680 | 1.00 | 0.00 | 0.053 | 0.00 | 0.00 |
| ATOM | 3511 | HG2 | VAL | 321 | 2.992 | 22.819 | 7.433 | 1.00 | 0.00 | 0.053 | 0.00 | 0.00 |
| ATOM | 3512 | HG2 | VAL | 321 | 3.232 | 21.146 | 7.991 | 1.00 | 0.00 | 0.053 | 0.00 | 0.00 |
| ATOM | 3513 | C | VAL | 321 | 6.254 | 23.296 | 9.830 | 1.00 | 0.00 | 0.396 | 9.82 | 4.00 |
| ATOM | 3514 | O | VAL | 321 | 6.976 | 24.247 | 9.512 | 1.00 | 0.00 | −0.396 | 8.17 | −17.40 |
| ATOM | 3515 | N | SER | 322 | 6.255 | 22.759 | 11.047 | 1.00 | 0.00 | −0.650 | 9.00 | −17.40 |
| ATOM | 3516 | HN | SER | 322 | 5.622 | 21.974 | 11.259 | 1.00 | 0.00 | 0.440 | 0.00 | 0.00 |
| ATOM | 3517 | CA | SER | 322 | 7.144 | 23.272 | 12.083 | 1.00 | 0.00 | 0.158 | 9.40 | 4.00 |
| ATOM | 3518 | HA | SER | 322 | 6.929 | 24.329 | 12.235 | 1.00 | 0.00 | 0.053 | 0.00 | 0.00 |
| ATOM | 3519 | CB | SER | 322 | 6.973 | 22.510 | 13.406 | 1.00 | 0.00 | 0.007 | 12.77 | 4.00 |
| ATOM | 3520 | HB1 | SER | 322 | 7.633 | 22.886 | 14.187 | 1.00 | 0.00 | 0.053 | 0.00 | 0.00 |
| ATOM | 3521 | HB2 | SER | 322 | 7.190 | 21.447 | 13.301 | 1.00 | 0.00 | 0.053 | 0.00 | 0.00 |
| ATOM | 3522 | OG | SER | 322 | 5.654 | 22.604 | 13.912 | 1.00 | 0.00 | −0.537 | 11.04 | −17.40 |
| ATOM | 3523 | HG | SER | 322 | 5.398 | 23.595 | 14.023 | 1.00 | 0.00 | 0.424 | 0.00 | 0.00 |
| ATOM | 3524 | C | SER | 322 | 8.582 | 23.088 | 11.627 | 1.00 | 0.00 | 0.396 | 9.82 | 4.00 |
| ATOM | 3525 | O | SER | 322 | 8.883 | 22.248 | 10.771 | 1.00 | 0.00 | −0.396 | 8.17 | −17.40 |
| ATOM | 3526 | N | HIS | 323S | 9.468 | 23.882 | 12.211 | 1.00 | 0.00 | −0.650 | 9.00 | −17.40 |
| ATOM | 3527 | HN | HIS | 323S | 9.144 | 24.581 | 12.894 | 1.00 | 0.00 | 0.440 | 0.00 | 0.00 |
| ATOM | 3528 | CA | HIS | 323S | 10.882 | 23.788 | 11.909 | 1.00 | 0.00 | 0.158 | 9.40 | 4.00 |
| ATOM | 3529 | HA | HIS | 323S | 10.950 | 23.686 | 10.826 | 1.00 | 0.00 | 0.053 | 0.00 | 0.00 |
| ATOM | 3530 | CB | HIS | 323S | 11.577 | 25.075 | 12.358 | 1.00 | 0.00 | −0.106 | 12.77 | 4.00 |
| ATOM | 3531 | HB1 | HIS | 323S | 12.618 | 25.131 | 12.040 | 1.00 | 0.00 | 0.053 | 0.00 | 0.00 |
| ATOM | 3532 | HB2 | HIS | 323S | 11.592 | 25.195 | 13.441 | 1.00 | 0.00 | 0.053 | 0.00 | 0.00 |
| ATOM | 3533 | CG | HIS | 323S | 10.923 | 26.313 | 11.822 | 1.00 | 0.00 | −0.050 | 7.26 | 0.60 |
| ATOM | 3534 | CD2 | HIS | 323S | 10.432 | 27.410 | 12.448 | 1.00 | 0.00 | −0.177 | 10.80 | 0.60 |
| ATOM | 3535 | HD2 | HIS | 323S | 10.458 | 27.609 | 13.519 | 1.00 | 0.00 | 0.127 | 0.00 | 0.00 |
| ATOM | 3536 | ND1 | HIS | 323S | 10.671 | 26.494 | 10.477 | 1.00 | 0.00 | 0.207 | 9.25 | −17.40 |
| ATOM | 3537 | HD1 | HIS | 323S | 10.925 | 25.833 | 9.729 | 1.00 | 0.00 | 0.393 | 0.00 | 0.00 |
| ATOM | 3538 | CE1 | HIS | 323S | 10.052 | 27.649 | 10.299 | 1.00 | 0.00 | −0.227 | 10.80 | 0.60 |
| ATOM | 3539 | HE1 | HIS | 323S | 9.727 | 28.056 | 9.342 | 1.00 | 0.00 | 0.127 | 0.00 | 0.00 |
| ATOM | 3540 | NE2 | HIS | 323S | 9.894 | 28.225 | 11.478 | 1.00 | 0.00 | 0.207 | 9.25 | −17.40 |
| ATOM | 3541 | HE2 | HIS | 323S | 9.441 | 29.135 | 11.644 | 1.00 | 0.00 | 0.393 | 0.00 | 0.00 |
| ATOM | 3542 | C | HIS | 323S | 11.392 | 22.543 | 12.658 | 1.00 | 0.00 | 0.396 | 9.82 | 4.00 |
| ATOM | 3543 | O | HIS | 323S | 10.748 | 22.069 | 13.603 | 1.00 | 0.00 | −0.396 | 8.17 | −17.40 |
| ATOM | 3544 | N | GLY | 324 | 12.534 | 22.013 | 12.231 | 1.00 | 0.00 | −0.650 | 9.00 | −17.40 |
| ATOM | 3545 | HN | GLY | 324 | 13.053 | 22.467 | 11.467 | 1.00 | 0.00 | 0.440 | 0.00 | 0.00 |
| ATOM | 3546 | CA | GLY | 324 | 13.054 | 20.799 | 12.833 | 1.00 | 0.00 | 0.105 | 9.40 | 4.00 |
| ATOM | 3547 | HA1 | GLY | 324 | 13.786 | 20.403 | 12.129 | 1.00 | 0.00 | 0.053 | 0.00 | 0.00 |
| ATOM | 3548 | HA2 | GLY | 324 | 12.198 | 20.137 | 12.965 | 1.00 | 0.00 | 0.053 | 0.00 | 0.00 |
| ATOM | 3549 | C | GLY | 324 | 13.766 | 20.773 | 14.178 | 1.00 | 0.00 | 0.396 | 9.82 | 4.00 |
| ATOM | 3550 | O | GLY | 324 | 14.357 | 19.738 | 14.502 | 1.00 | 0.00 | −0.396 | 8.17 | −17.40 |
| ATOM | 3551 | N | GLN | 325 | 13.728 | 21.843 | 14.973 | 1.00 | 0.00 | −0.650 | 9.00 | −17.40 |
| ATOM | 3552 | HN | GLN | 325 | 13.209 | 22.688 | 14.693 | 1.00 | 0.00 | 0.440 | 0.00 | 0.00 |
| ATOM | 3553 | CA | GLN | 325 | 14.439 | 21.791 | 16.253 | 1.00 | 0.00 | 0.158 | 9.40 | 4.00 |
| ATOM | 3554 | HA | GLN | 325 | 15.457 | 21.450 | 16.067 | 1.00 | 0.00 | 0.053 | 0.00 | 0.00 |
| ATOM | 3555 | CB | GLN | 325 | 14.596 | 23.177 | 16.878 | 1.00 | 0.00 | −0.106 | 12.77 | 4.00 |
| ATOM | 3556 | HB1 | GLN | 325 | 13.635 | 23.616 | 17.146 | 1.00 | 0.00 | 0.053 | 0.00 | 0.00 |
| ATOM | 3557 | HB2 | GLN | 325 | 15.084 | 23.878 | 16.201 | 1.00 | 0.00 | 0.053 | 0.00 | 0.00 |
| ATOM | 3558 | CG | GLN | 325 | 15.436 | 23.139 | 18.156 | 1.00 | 0.00 | −0.106 | 12.77 | 4.00 |
| ATOM | 3559 | HG1 | GLN | 325 | 16.217 | 22.379 | 18.140 | 1.00 | 0.00 | 0.053 | 0.00 | 0.00 |
| ATOM | 3560 | HG2 | GLN | 325 | 14.853 | 22.930 | 19.053 | 1.00 | 0.00 | 0.053 | 0.00 | 0.00 |
| ATOM | 3561 | CD | GLN | 325 | 16.160 | 24.449 | 18.447 | 1.00 | 0.00 | 0.396 | 9.82 | 4.00 |
| ATOM | 3562 | OE1 | GLN | 325 | 15.533 | 25.478 | 18.719 | 1.00 | 0.00 | −0.396 | 8.17 | −17.40 |
| ATOM | 3563 | NE2 | GLN | 325 | 17.492 | 24.415 | 18.384 | 1.00 | 0.00 | −0.879 | 13.25 | −17.40 |
| ATOM | 3564 | HE2 | GLN | 325 | 17.974 | 23.534 | 18.153 | 1.00 | 0.00 | 0.440 | 0.00 | 0.00 |
| ATOM | 3565 | HE2 | GLN | 325 | 18.038 | 25.269 | 18.565 | 1.00 | 0.00 | 0.440 | 0.00 | 0.00 |
| ATOM | 3566 | C | GLN | 325 | 13.811 | 20.848 | 17.271 | 1.00 | 0.00 | 0.396 | 9.82 | 4.00 |
| ATOM | 3567 | O | GLN | 325 | 14.502 | 20.013 | 17.863 | 1.00 | 0.00 | −0.396 | 8.17 | −17.40 |
| ATOM | 3568 | N | PHE | 326 | 12.509 | 20.976 | 17.485 | 1.00 | 0.00 | −0.650 | 9.00 | −17.40 |
| ATOM | 3569 | HN | PHE | 326 | 11.976 | 21.696 | 16.977 | 1.00 | 0.00 | 0.440 | 0.00 | 0.00 |
| ATOM | 3570 | CA | PHE | 326 | 11.829 | 20.109 | 18.428 | 1.00 | 0.00 | 0.158 | 9.40 | 4.00 |
| ATOM | 3571 | HA | PHE | 326 | 12.285 | 20.212 | 19.412 | 1.00 | 0.00 | 0.053 | 0.00 | 0.00 |
| ATOM | 3572 | CB | PHE | 326 | 10.357 | 20.511 | 18.564 | 1.00 | 0.00 | −0.106 | 12.77 | 4.00 |
| ATOM | 3573 | HB1 | PHE | 326 | 9.765 | 20.309 | 17.670 | 1.00 | 0.00 | 0.053 | 0.00 | 0.00 |
| ATOM | 3574 | HB2 | PHE | 326 | 10.211 | 21.572 | 18.767 | 1.00 | 0.00 | 0.053 | 0.00 | 0.00 |
| ATOM | 3575 | CG | PHE | 326 | 9.642 | 19.801 | 19.672 | 1.00 | 0.00 | 0.000 | 7.26 | 0.60 |
| ATOM | 3576 | CD1 | PHE | 326 | 10.205 | 19.736 | 20.947 | 1.00 | 0.00 | −0.127 | 10.80 | 0.60 |
| ATOM | 3577 | HD1 | PHE | 326 | 11.155 | 20.233 | 21.140 | 1.00 | 0.00 | 0.127 | 0.00 | 0.00 |
| ATOM | 3578 | CD2 | PHE | 326 | 8.427 | 19.169 | 19.443 | 1.00 | 0.00 | −0.127 | 10.80 | 0.60 |
| ATOM | 3579 | HD2 | PHE | 326 | 7.972 | 19.216 | 18.453 | 1.00 | 0.00 | 0.127 | 0.00 | 0.00 |
| ATOM | 3580 | CE1 | PHE | 326 | 9.576 | 19.050 | 21.968 | 1.00 | 0.00 | −0.127 | 10.80 | 0.60 |
| ATOM | 3581 | HE1 | PHE | 326 | 10.028 | 19.005 | 22.958 | 1.00 | 0.00 | 0.127 | 0.00 | 0.00 |
| ATOM | 3582 | CE2 | PHE | 326 | 7.788 | 18.480 | 20.459 | 1.00 | 0.00 | −0.127 | 10.80 | 0.60 |
| ATOM | 3583 | HE2 | PHE | 326 | 6.834 | 17.987 | 20.267 | 1.00 | 0.00 | 0.127 | 0.00 | 0.00 |
| ATOM | 3584 | CZ | PHE | 326 | 8.361 | 18.416 | 21.724 | 1.00 | 0.00 | −0.127 | 10.80 | 0.60 |
| ATOM | 3585 | HZ | PHE | 326 | 7.859 | 17.870 | 22.523 | 1.00 | 0.00 | 0.127 | 0.00 | 0.00 |
| ATOM | 3586 | C | PHE | 326 | 11.946 | 18.659 | 17.964 | 1.00 | 0.00 | 0.396 | 9.82 | 4.00 |
| ATOM | 3587 | O | PHE | 326 | 12.147 | 17.757 | 18.786 | 1.00 | 0.00 | −0.396 | 8.17 | −17.40 |
| ATOM | 3588 | N | PHE | 327 | 11.847 | 18.437 | 16.653 | 1.00 | 0.00 | −0.650 | 9.00 | −17.40 |
| ATOM | 3589 | HN | PHE | 327 | 11.684 | 19.232 | 16.019 | 1.00 | 0.00 | 0.440 | 0.00 | 0.00 |
| ATOM | 3590 | CA | PHE | 327 | 11.964 | 17.086 | 16.092 | 1.00 | 0.00 | 0.158 | 9.40 | 4.00 |
| ATOM | 3591 | HA | PHE | 327 | 11.279 | 16.405 | 16.598 | 1.00 | 0.00 | 0.053 | 0.00 | 0.00 |
| ATOM | 3592 | CB | PHE | 327 | 11.633 | 17.061 | 14.584 | 1.00 | 0.00 | −0.106 | 12.77 | 4.00 |
| ATOM | 3593 | HB1 | PHE | 327 | 12.193 | 17.794 | 14.004 | 1.00 | 0.00 | 0.053 | 0.00 | 0.00 |
| ATOM | 3594 | HB2 | PHE | 327 | 10.584 | 17.265 | 14.365 | 1.00 | 0.00 | 0.053 | 0.00 | 0.00 |
| ATOM | 3595 | CG | PHE | 327 | 11.923 | 15.726 | 13.923 | 1.00 | 0.00 | 0.000 | 7.26 | 0.60 |
| ATOM | 3596 | CD1 | PHE | 327 | 10.920 | 14.762 | 13.796 | 1.00 | 0.00 | −0.127 | 10.80 | 0.60 |
| ATOM | 3597 | HD1 | PHE | 327 | 9.902 | 15.003 | 14.101 | 1.00 | 0.00 | 0.127 | 0.00 | 0.00 |
| ATOM | 3598 | CD2 | PHE | 327 | 13.222 | 15.402 | 13.519 | 1.00 | 0.00 | −0.127 | 10.80 | 0.60 |
| ATOM | 3599 | HD2 | PHE | 327 | 14.011 | 16.148 | 13.606 | 1.00 | 0.00 | 0.127 | 0.00 | 0.00 |
| ATOM | 3600 | CE1 | PHE | 327 | 11.202 | 13.488 | 13.282 | 1.00 | 0.00 | −0.127 | 10.80 | 0.60 |
| ATOM | 3601 | HE1 | PHE | 327 | 10.408 | 12.746 | 13.190 | 1.00 | 0.00 | 0.127 | 0.00 | 0.00 |
| ATOM | 3602 | CE2 | PHE | 327 | 13.526 | 14.135 | 13.004 | 1.00 | 0.00 | −0.127 | 10.80 | 0.60 |
| ATOM | 3603 | HE2 | PHE | 327 | 14.544 | 13.898 | 12.696 | 1.00 | 0.00 | 0.127 | 0.00 | 0.00 |
| ATOM | 3604 | CZ | PHE | 327 | 12.512 | 13.171 | 12.886 | 1.00 | 0.00 | −0.127 | 10.80 | 0.60 |
| ATOM | 3605 | HZ | PHE | 327 | 12.740 | 12.181 | 12.489 | 1.00 | 0.00 | 0.127 | 0.00 | 0.00 |
| ATOM | 3606 | C | PHE | 327 | 13.385 | 16.564 | 16.263 | 1.00 | 0.00 | 0.396 | 9.82 | 4.00 |
| ATOM | 3607 | O | PHE | 327 | 13.592 | 15.379 | 16.554 | 1.00 | 0.00 | −0.396 | 8.17 | −17.40 |
| ATOM | 3608 | N | ASP | 328P | 14.358 | 17.453 | 16.073 | 1.00 | 0.00 | −0.650 | 9.00 | −17.40 |
| ATOM | 3609 | HN | ASP | 328P | 14.106 | 18.430 | 15.866 | 1.00 | 0.00 | 0.440 | 0.00 | 0.00 |
| ATOM | 3610 | CA | ASP | 328P | 15.766 | 17.090 | 16.148 | 1.00 | 0.00 | 0.158 | 9.40 | 4.00 |
| ATOM | 3611 | HA | ASP | 328P | 15.907 | 16.173 | 15.575 | 1.00 | 0.00 | 0.053 | 0.00 | 0.00 |
| ATOM | 3612 | CB | ASP | 328P | 16.637 | 18.167 | 15.483 | 1.00 | 0.00 | −0.336 | 12.77 | 4.00 |
| ATOM | 3613 | HB1 | ASP | 328P | 17.620 | 18.261 | 15.943 | 1.00 | 0.00 | 0.053 | 0.00 | 0.00 |
| ATOM | 3614 | HB2 | ASP | 328P | 16.191 | 19.160 | 15.528 | 1.00 | 0.00 | 0.053 | 0.00 | 0.00 |
| ATOM | 3615 | CG | ASP | 328P | 16.895 | 17.893 | 13.999 | 1.00 | 0.00 | 0.297 | 9.82 | 4.00 |
| ATOM | 3616 | OD1 | ASP | 328P | 16.766 | 16.723 | 13.555 | 1.00 | 0.00 | −0.534 | 8.17 | −18.95 |
| ATOM | 3617 | OD2 | ASP | 328P | 17.254 | 18.855 | 13.273 | 1.00 | 0.00 | −0.534 | 8.17 | −18.95 |
| ATOM | 3618 | C | ASP | 328P | 16.306 | 16.834 | 17.545 | 1.00 | 0.00 | 0.396 | 9.82 | 4.00 |
| ATOM | 3619 | O | ASP | 328P | 17.203 | 16.003 | 17.731 | 1.00 | 0.00 | −0.396 | 8.17 | −17.40 |
| ATOM | 3620 | N | GLN | 329 | 15.765 | 17.537 | 18.529 | 1.00 | 0.00 | −0.650 | 9.00 | −17.40 |
| ATOM | 3621 | HN | GLN | 329 | 14.994 | 18.192 | 18.333 | 1.00 | 0.00 | 0.440 | 0.00 | 0.00 |
| ATOM | 3622 | CA | GLN | 329 | 16.262 | 17.381 | 19.886 | 1.00 | 0.00 | 0.158 | 9.40 | 4.00 |
| ATOM | 3623 | HA | GLN | 329 | 17.258 | 16.943 | 19.836 | 1.00 | 0.00 | 0.053 | 0.00 | 0.00 |
| ATOM | 3624 | CB | GLN | 329 | 16.415 | 18.748 | 20.534 | 1.00 | 0.00 | −0.106 | 12.77 | 4.00 |
| ATOM | 3625 | HB1 | GLN | 329 | 16.656 | 18.690 | 21.595 | 1.00 | 0.00 | 0.053 | 0.00 | 0.00 |
| ATOM | 3626 | HB2 | GLN | 329 | 15.511 | 19.353 | 20.468 | 1.00 | 0.00 | 0.053 | 0.00 | 0.00 |
| ATOM | 3627 | CG | GLN | 329 | 17.513 | 19.579 | 19.907 | 1.00 | 0.00 | −0.106 | 12.77 | 4.00 |
| ATOM | 3628 | HG1 | GLN | 329 | 17.257 | 19.882 | 18.891 | 1.00 | 0.00 | 0.053 | 0.00 | 0.00 |
| ATOM | 3629 | HG2 | GLN | 329 | 18.452 | 19.029 | 19.852 | 1.00 | 0.00 | 0.053 | 0.00 | 0.00 |
| ATOM | 3630 | CD | GLN | 329 | 17.778 | 20.835 | 20.694 | 1.00 | 0.00 | 0.396 | 9.82 | 4.00 |
| ATOM | 3631 | OE1 | GLN | 329 | 16.900 | 21.690 | 20.827 | 1.00 | 0.00 | −0.396 | 8.17 | −17.40 |
| ATOM | 3632 | NE2 | GLN | 329 | 18.989 | 20.956 | 21.231 | 1.00 | 0.00 | −0.879 | 13.25 | −17.40 |
| ATOM | 3633 | HE2 | GLN | 329 | 19.690 | 20.214 | 21.092 | 1.00 | 0.00 | 0.440 | 0.00 | 0.00 |
| ATOM | 3634 | HE2 | GLN | 329 | 19.225 | 21.791 | 21.785 | 1.00 | 0.00 | 0.440 | 0.00 | 0.00 |
| ATOM | 3635 | C | GLN | 329 | 15.414 | 16.489 | 20.766 | 1.00 | 0.00 | 0.396 | 9.82 | 4.00 |
| ATOM | 3636 | O | GLN | 329 | 15.855 | 16.067 | 21.835 | 1.00 | 0.00 | −0.396 | 8.17 | −17.40 |
| ATOM | 3637 | N | HIS | 330S | 14.212 | 16.172 | 20.308 | 1.00 | 0.00 | −0.650 | 9.00 | −17.40 |
| ATOM | 3638 | HN | HIS | 330S | 13.897 | 16.513 | 19.388 | 1.00 | 0.00 | 0.440 | 0.00 | 0.00 |
| ATOM | 3639 | CA | HIS | 330S | 13.346 | 15.339 | 21.118 | 1.00 | 0.00 | 0.158 | 9.40 | 4.00 |
| ATOM | 3640 | HA | HIS | 330S | 13.973 | 14.821 | 21.843 | 1.00 | 0.00 | 0.053 | 0.00 | 0.00 |
| ATOM | 3641 | CB | HIS | 330S | 12.387 | 16.225 | 21.908 | 1.00 | 0.00 | −0.106 | 12.77 | 4.00 |
| ATOM | 3642 | HB1 | HIS | 330S | 11.748 | 16.843 | 21.277 | 1.00 | 0.00 | 0.053 | 0.00 | 0.00 |
| ATOM | 3643 | HB2 | HIS | 330S | 12.891 | 16.923 | 22.575 | 1.00 | 0.00 | 0.053 | 0.00 | 0.00 |
| ATOM | 3644 | CG | HIS | 330S | 11.449 | 15.456 | 22.782 | 1.00 | 0.00 | −0.050 | 7.26 | 0.60 |
| ATOM | 3645 | CD2 | HIS | 330S | 10.127 | 15.184 | 22.649 | 1.00 | 0.00 | −0.177 | 10.80 | 0.60 |
| ATOM | 3646 | HD2 | HIS | 330S | 9.471 | 15.531 | 21.850 | 1.00 | 0.00 | 0.127 | 0.00 | 0.00 |
| ATOM | 3647 | ND1 | HIS | 330S | 11.861 | 14.815 | 23.934 | 1.00 | 0.00 | 0.207 | 9.25 | −17.40 |
| ATOM | 3648 | HD1 | HIS | 330S | 12.818 | 14.828 | 24.314 | 1.00 | 0.00 | 0.393 | 0.00 | 0.00 |
| ATOM | 3649 | CE1 | HIS | 330S | 10.833 | 14.181 | 24.472 | 1.00 | 0.00 | −0.227 | 10.80 | 0.60 |
| ATOM | 3650 | HE1 | HIS | 330S | 10.857 | 13.588 | 25.386 | 1.00 | 0.00 | 0.127 | 0.00 | 0.00 |
| ATOM | 3651 | NE2 | HIS | 330S | 9.769 | 14.388 | 23.711 | 1.00 | 0.00 | 0.207 | 9.25 | −17.40 |
| ATOM | 3652 | HE2 | HIS | 330S | 8.825 | 14.013 | 23.886 | 1.00 | 0.00 | 0.393 | 0.00 | 0.00 |
| ATOM | 3653 | C | HIS | 330S | 12.530 | 14.261 | 20.400 | 1.00 | 0.00 | 0.396 | 9.82 | 4.00 |
| ATOM | 3654 | O | HIS | 330S | 12.694 | 13.069 | 20.667 | 1.00 | 0.00 | −0.396 | 8.17 | −17.40 |
| ATOM | 3655 | N | LEU | 331 | 11.657 | 14.687 | 19.490 | 1.00 | 0.00 | −0.650 | 9.00 | −17.40 |
| ATOM | 3656 | HN | LEU | 331 | 11.605 | 15.692 | 19.274 | 1.00 | 0.00 | 0.440 | 0.00 | 0.00 |
| ATOM | 3657 | CA | LEU | 331 | 10.777 | 13.769 | 18.795 | 1.00 | 0.00 | 0.158 | 9.40 | 4.00 |
| ATOM | 3658 | HA | LEU | 331 | 10.081 | 13.361 | 19.528 | 1.00 | 0.00 | 0.053 | 0.00 | 0.00 |
| ATOM | 3659 | CB | LEU | 331 | 9.901 | 14.529 | 17.798 | 1.00 | 0.00 | −0.106 | 12.77 | 4.00 |
| ATOM | 3660 | HB1 | LEU | 331 | 9.291 | 13.794 | 17.272 | 1.00 | 0.00 | 0.053 | 0.00 | 0.00 |
| ATOM | 3661 | HB2 | LEU | 331 | 10.566 | 15.057 | 17.114 | 1.00 | 0.00 | 0.053 | 0.00 | 0.00 |
| ATOM | 3662 | CG | LEU | 331 | 8.928 | 15.581 | 18.359 | 1.00 | 0.00 | −0.053 | 9.40 | 4.00 |
| ATOM | 3663 | HG | LEU | 331 | 9.512 | 16.310 | 18.920 | 1.00 | 0.00 | 0.053 | 0.00 | 0.00 |
| ATOM | 3664 | CD1 | LEU | 331 | 8.225 | 16.232 | 17.188 | 1.00 | 0.00 | −0.159 | 16.15 | 4.00 |
| ATOM | 3665 | HD1 | LEU | 331 | 7.526 | 16.984 | 17.555 | 1.00 | 0.00 | 0.053 | 0.00 | 0.00 |
| ATOM | 3666 | HD1 | LEU | 331 | 8.961 | 16.706 | 16.539 | 1.00 | 0.00 | 0.053 | 0.00 | 0.00 |
| ATOM | 3667 | HD1 | LEU | 331 | 7.679 | 15.475 | 16.624 | 1.00 | 0.00 | 0.053 | 0.00 | 0.00 |
| ATOM | 3668 | CD2 | LEU | 331 | 7.913 | 14.967 | 19.305 | 1.00 | 0.00 | −0.159 | 16.15 | 4.00 |
| ATOM | 3669 | HD2 | LEU | 331 | 7.246 | 15.744 | 19.677 | 1.00 | 0.00 | 0.053 | 0.00 | 0.00 |
| ATOM | 3670 | HD2 | LEU | 331 | 7.331 | 14.213 | 18.775 | 1.00 | 0.00 | 0.053 | 0.00 | 0.00 |
| ATOM | 3671 | HD2 | LEU | 331 | 8.431 | 14.501 | 20.143 | 1.00 | 0.00 | 0.053 | 0.00 | 0.00 |
| ATOM | 3672 | C | LEU | 331 | 11.420 | 12.582 | 18.100 | 1.00 | 0.00 | 0.396 | 9.82 | 4.00 |
| ATOM | 3673 | O | LEU | 331 | 10.867 | 11.480 | 18.147 | 1.00 | 0.00 | −0.396 | 8.17 | −17.40 |
| ATOM | 3674 | N | LYS | 332S | 12.574 | 12.795 | 17.465 | 1.00 | 0.00 | −0.650 | 9.00 | −17.40 |
| ATOM | 3675 | HN | LYS | 332S | 13.000 | 13.732 | 17.487 | 1.00 | 0.00 | 0.440 | 0.00 | 0.00 |
| ATOM | 3676 | CA | LYS | 332S | 13.243 | 11.714 | 16.736 | 1.00 | 0.00 | 0.158 | 9.40 | 4.00 |
| ATOM | 3677 | HA | LYS | 332S | 12.584 | 11.219 | 16.022 | 1.00 | 0.00 | 0.053 | 0.00 | 0.00 |
| ATOM | 3678 | CB | LYS | 332S | 14.422 | 12.261 | 15.897 | 1.00 | 0.00 | −0.106 | 12.77 | 4.00 |
| ATOM | 3679 | HB1 | LYS | 332S | 14.058 | 13.106 | 15.313 | 1.00 | 0.00 | 0.053 | 0.00 | 0.00 |
| ATOM | 3680 | HB2 | LYS | 332S | 14.772 | 11.461 | 15.243 | 1.00 | 0.00 | 0.053 | 0.00 | 0.00 |
| ATOM | 3681 | CG | LYS | 332S | 15.646 | 12.757 | 16.724 | 1.00 | 0.00 | −0.106 | 12.77 | 4.00 |
| ATOM | 3682 | HG1 | LYS | 332S | 16.079 | 11.977 | 17.350 | 1.00 | 0.00 | 0.053 | 0.00 | 0.00 |
| ATOM | 3683 | HG2 | LYS | 332S | 15.399 | 13.576 | 17.398 | 1.00 | 0.00 | 0.053 | 0.00 | 0.00 |
| ATOM | 3684 | CD | LYS | 332S | 16.810 | 13.279 | 15.832 | 1.00 | 0.00 | −0.106 | 12.77 | 4.00 |
| ATOM | 3685 | HD1 | LYS | 332S | 17.462 | 13.945 | 16.396 | 1.00 | 0.00 | 0.053 | 0.00 | 0.00 |
| ATOM | 3686 | HD2 | LYS | 332S | 16.424 | 13.830 | 14.974 | 1.00 | 0.00 | 0.053 | 0.00 | 0.00 |
| ATOM | 3687 | CE | LYS | 332S | 17.652 | 12.125 | 15.310 | 1.00 | 0.00 | 0.099 | 12.77 | 4.00 |
| ATOM | 3688 | HE1 | LYS | 332S | 17.050 | 11.551 | 14.604 | 1.00 | 0.00 | 0.053 | 0.00 | 0.00 |
| ATOM | 3689 | HE2 | LYS | 332S | 17.945 | 11.503 | 16.156 | 1.00 | 0.00 | 0.053 | 0.00 | 0.00 |
| ATOM | 3690 | NZ | LYS | 332S | 18.904 | 12.555 | 14.601 | 1.00 | 0.00 | −0.045 | 13.25 | −39.20 |
| ATOM | 3691 | HZ1 | LYS | 332S | 19.418 | 11.723 | 14.278 | 1.00 | 0.00 | 0.280 | 0.00 | 0.00 |
| ATOM | 3692 | HZ2 | LYS | 332S | 18.656 | 13.141 | 13.791 | 1.00 | 0.00 | 0.280 | 0.00 | 0.00 |
| ATOM | 3693 | HZ3 | LYS | 332S | 19.496 | 13.095 | 15.247 | 1.00 | 0.00 | 0.280 | 0.00 | 0.00 |
| ATOM | 3694 | C | LYS | 332S | 13.764 | 10.621 | 17.659 | 1.00 | 0.00 | 0.396 | 9.82 | 4.00 |
| ATOM | 3695 | O | LYS | 332S | 14.106 | 9.541 | 17.201 | 1.00 | 0.00 | −0.396 | 8.17 | −17.40 |
| ATOM | 3696 | N | PHE | 333 | 13.824 | 10.893 | 18.955 | 1.00 | 0.00 | −0.650 | 9.00 | −17.40 |
| ATOM | 3697 | HN | PHE | 333 | 13.492 | 11.801 | 19.309 | 1.00 | 0.00 | 0.440 | 0.00 | 0.00 |
| ATOM | 3698 | CA | PHE | 333 | 14.363 | 9.897 | 19.879 | 1.00 | 0.00 | 0.158 | 9.40 | 4.00 |
| ATOM | 3699 | HA | PHE | 333 | 15.045 | 9.255 | 19.322 | 1.00 | 0.00 | 0.053 | 0.00 | 0.00 |
| ATOM | 3700 | CB | PHE | 333 | 15.223 | 10.592 | 20.943 | 1.00 | 0.00 | −0.106 | 12.77 | 4.00 |
| ATOM | 3701 | HB1 | PHE | 333 | 15.616 | 9.900 | 21.687 | 1.00 | 0.00 | 0.053 | 0.00 | 0.00 |
| ATOM | 3702 | HB2 | PHE | 333 | 14.674 | 11.350 | 21.501 | 1.00 | 0.00 | 0.053 | 0.00 | 0.00 |
| ATOM | 3703 | CG | PHE | 333 | 16.416 | 11.292 | 20.362 | 1.00 | 0.00 | 0.000 | 7.26 | 0.60 |
| ATOM | 3704 | CD1 | PHE | 333 | 16.505 | 12.681 | 20.382 | 1.00 | 0.00 | −0.127 | 10.80 | 0.60 |
| ATOM | 3705 | HD1 | PHE | 333 | 15.729 | 13.262 | 20.880 | 1.00 | 0.00 | 0.127 | 0.00 | 0.00 |
| ATOM | 3706 | CD2 | PHE | 333 | 17.420 | 10.556 | 19.726 | 1.00 | 0.00 | −0.127 | 10.80 | 0.60 |
| ATOM | 3707 | HD2 | PHE | 333 | 17.361 | 9.467 | 19.711 | 1.00 | 0.00 | 0.127 | 0.00 | 0.00 |
| ATOM | 3708 | CE1 | PHE | 333 | 17.577 | 13.340 | 19.770 | 1.00 | 0.00 | −0.127 | 10.80 | 0.60 |
| ATOM | 3709 | HE1 | PHE | 333 | 17.634 | 14.428 | 19.792 | 1.00 | 0.00 | 0.127 | 0.00 | 0.00 |
| ATOM | 3710 | CE2 | PHE | 333 | 18.498 | 11.197 | 19.109 | 1.00 | 0.00 | −0.127 | 10.80 | 0.60 |
| ATOM | 3711 | HE2 | PHE | 333 | 19.275 | 10.613 | 18.615 | 1.00 | 0.00 | 0.127 | 0.00 | 0.00 |
| ATOM | 3712 | CZ | PHE | 333 | 18.571 | 12.600 | 19.131 | 1.00 | 0.00 | −0.127 | 10.80 | 0.60 |
| ATOM | 3713 | HZ | PHE | 333 | 19.404 | 13.109 | 18.648 | 1.00 | 0.00 | 0.127 | 0.00 | 0.00 |
| ATOM | 3714 | C | PHE | 333 | 13.335 | 8.995 | 20.540 | 1.00 | 0.00 | 0.396 | 9.82 | 4.00 |
| ATOM | 3715 | O | PHE | 333 | 13.700 | 8.057 | 21.252 | 1.00 | 0.00 | −0.396 | 8.17 | −17.40 |
| ATOM | 3716 | N | ILE | 334 | 12.055 | 9.262 | 20.286 | 1.00 | 0.00 | −0.650 | 9.00 | −17.40 |
| ATOM | 3717 | HN | ILE | 334 | 11.815 | 10.052 | 19.670 | 1.00 | 0.00 | 0.440 | 0.00 | 0.00 |
| ATOM | 3718 | CA | ILE | 334 | 10.988 | 8.457 | 20.862 | 1.00 | 0.00 | 0.158 | 9.40 | 4.00 |
| ATOM | 3719 | HA | ILE | 334 | 11.190 | 8.351 | 21.928 | 1.00 | 0.00 | 0.053 | 0.00 | 0.00 |
| ATOM | 3720 | CB | ILE | 334 | 9.624 | 9.176 | 20.700 | 1.00 | 0.00 | −0.053 | 9.40 | 4.00 |
| ATOM | 3721 | HB | ILE | 334 | 9.401 | 9.368 | 19.650 | 1.00 | 0.00 | 0.053 | 0.00 | 0.00 |
| ATOM | 3722 | CG2 | ILE | 334 | 8.493 | 8.340 | 21.264 | 1.00 | 0.00 | −0.159 | 16.15 | 4.00 |
| ATOM | 3723 | HG2 | ILE | 334 | 7.549 | 8.870 | 21.135 | 1.00 | 0.00 | 0.053 | 0.00 | 0.00 |
| ATOM | 3724 | HG2 | ILE | 334 | 8.447 | 7.386 | 20.737 | 1.00 | 0.00 | 0.053 | 0.00 | 0.00 |
| ATOM | 3725 | HG2 | ILE | 334 | 8.667 | 8.160 | 22.325 | 1.00 | 0.00 | 0.053 | 0.00 | 0.00 |
| ATOM | 3726 | CG1 | ILE | 334 | 9.691 | 10.528 | 21.417 | 1.00 | 0.00 | −0.106 | 12.77 | 4.00 |
| ATOM | 3727 | HG1 | ILE | 334 | 8.694 | 10.970 | 21.404 | 1.00 | 0.00 | 0.053 | 0.00 | 0.00 |
| ATOM | 3728 | HG1 | ILE | 334 | 10.400 | 11.161 | 20.884 | 1.00 | 0.00 | 0.053 | 0.00 | 0.00 |
| ATOM | 3729 | CD1 | ILE | 334 | 10.142 | 10.456 | 22.871 | 1.00 | 0.00 | −0.159 | 16.15 | 4.00 |
| ATOM | 3730 | HD1 | ILE | 334 | 10.159 | 11.459 | 23.296 | 1.00 | 0.00 | 0.053 | 0.00 | 0.00 |
| ATOM | 3731 | HD1 | ILE | 334 | 9.448 | 9.833 | 23.436 | 1.00 | 0.00 | 0.053 | 0.00 | 0.00 |
| ATOM | 3732 | HD1 | ILE | 334 | 11.141 | 10.023 | 22.921 | 1.00 | 0.00 | 0.053 | 0.00 | 0.00 |
| ATOM | 3733 | C | ILE | 334 | 11.000 | 7.101 | 20.162 | 1.00 | 0.00 | 0.396 | 9.82 | 4.00 |
| ATOM | 3734 | O | ILE | 334 | 10.825 | 7.015 | 18.941 | 1.00 | 0.00 | −0.396 | 8.17 | −17.40 |
| ATOM | 3735 | N | LYS | 335S | 11.215 | 6.048 | 20.946 | 1.00 | 0.00 | −0.650 | 9.00 | −17.40 |
| ATOM | 3736 | HN | LYS | 335S | 11.325 | 6.199 | 21.959 | 1.00 | 0.00 | 0.440 | 0.00 | 0.00 |
| ATOM | 3737 | CA | LYS | 335S | 11.301 | 4.687 | 20.426 | 1.00 | 0.00 | 0.158 | 9.40 | 4.00 |
| ATOM | 3738 | HA | LYS | 335S | 11.908 | 4.718 | 19.521 | 1.00 | 0.00 | 0.053 | 0.00 | 0.00 |
| ATOM | 3739 | CB | LYS | 335S | 12.043 | 3.800 | 21.433 | 1.00 | 0.00 | −0.106 | 12.77 | 4.00 |
| ATOM | 3740 | HB1 | LYS | 335S | 11.533 | 3.711 | 22.392 | 1.00 | 0.00 | 0.053 | 0.00 | 0.00 |
| ATOM | 3741 | HB2 | LYS | 335S | 13.043 | 4.158 | 21.674 | 1.00 | 0.00 | 0.053 | 0.00 | 0.00 |
| ATOM | 3742 | CG | LYS | 335S | 12.240 | 2.362 | 20.951 | 1.00 | 0.00 | −0.106 | 12.77 | 4.00 |
| ATOM | 3743 | HG1 | LYS | 335S | 12.912 | 2.294 | 20.095 | 1.00 | 0.00 | 0.053 | 0.00 | 0.00 |
| ATOM | 3744 | HG2 | LYS | 335S | 11.307 | 1.891 | 20.640 | 1.00 | 0.00 | 0.053 | 0.00 | 0.00 |
| ATOM | 3745 | CD | LYS | 335S | 12.833 | 1.456 | 22.029 | 1.00 | 0.00 | −0.106 | 12.77 | 4.00 |
| ATOM | 3746 | HD1 | LYS | 335S | 12.154 | 1.362 | 22.876 | 1.00 | 0.00 | 0.053 | 0.00 | 0.00 |
| ATOM | 3747 | HD2 | LYS | 335S | 13.777 | 1.855 | 22.398 | 1.00 | 0.00 | 0.053 | 0.00 | 0.00 |
| ATOM | 3748 | CE | LYS | 335S | 13.098 | 0.052 | 21.480 | 1.00 | 0.00 | 0.099 | 12.77 | 4.00 |
| ATOM | 3749 | HE1 | LYS | 335S | 12.172 | −0.454 | 21.206 | 1.00 | 0.00 | 0.053 | 0.00 | 0.00 |
| ATOM | 3750 | HE2 | LYS | 335S | 13.605 | −0.577 | 22.211 | 1.00 | 0.00 | 0.053 | 0.00 | 0.00 |
| ATOM | 3751 | NZ | LYS | 335S | 13.960 | 0.090 | 20.255 | 1.00 | 0.00 | −0.045 | 13.25 | −39.20 |
| ATOM | 3752 | HZ1 | LYS | 335S | 14.116 | −0.869 | 19.915 | 1.00 | 0.00 | 0.280 | 0.00 | 0.00 |
| ATOM | 3753 | HZ2 | LYS | 335S | 13.491 | 0.641 | 19.522 | 1.00 | 0.00 | 0.280 | 0.00 | 0.00 |
| ATOM | 3754 | HZ3 | LYS | 335S | 14.865 | 0.524 | 20.485 | 1.00 | 0.00 | 0.280 | 0.00 | 0.00 |
| ATOM | 3755 | C | LYS | 335S | 9.986 | 3.992 | 20.045 | 1.00 | 0.00 | 0.396 | 9.82 | 4.00 |
| ATOM | 3756 | O | LYS | 335S | 8.993 | 4.029 | 20.783 | 1.00 | 0.00 | −0.396 | 8.17 | −17.40 |
| ATOM | 3757 | N | LEU | 336 | 9.998 | 3.333 | 18.891 | 1.00 | 0.00 | −0.650 | 9.00 | −17.40 |
| ATOM | 3758 | HN | LEU | 336 | 10.848 | 3.344 | 18.310 | 1.00 | 0.00 | 0.440 | 0.00 | 0.00 |
| ATOM | 3759 | CA | LEU | 336 | 8.826 | 2.593 | 18.433 | 1.00 | 0.00 | 0.158 | 9.40 | 4.00 |
| ATOM | 3760 | HA | LEU | 336 | 7.907 | 3.109 | 18.712 | 1.00 | 0.00 | 0.053 | 0.00 | 0.00 |
| ATOM | 3761 | CB | LEU | 336 | 8.850 | 2.448 | 16.903 | 1.00 | 0.00 | −0.106 | 12.77 | 4.00 |
| ATOM | 3762 | HB1 | LEU | 336 | 9.711 | 1.832 | 16.642 | 1.00 | 0.00 | 0.053 | 0.00 | 0.00 |
| ATOM | 3763 | HB2 | LEU | 336 | 8.936 | 3.447 | 16.476 | 1.00 | 0.00 | 0.053 | 0.00 | 0.00 |
| ATOM | 3764 | CG | LEU | 336 | 7.618 | 1.790 | 16.250 | 1.00 | 0.00 | −0.053 | 9.40 | 4.00 |
| ATOM | 3765 | HG | LEU | 336 | 7.510 | 0.773 | 16.629 | 1.00 | 0.00 | 0.053 | 0.00 | 0.00 |
| ATOM | 3766 | CD1 | LEU | 336 | 6.381 | 2.597 | 16.592 | 1.00 | 0.00 | −0.159 | 16.15 | 4.00 |
| ATOM | 3767 | HD1 | LEU | 336 | 5.507 | 2.135 | 16.132 | 1.00 | 0.00 | 0.053 | 0.00 | 0.00 |
| ATOM | 3768 | HD1 | LEU | 336 | 6.251 | 2.622 | 17.674 | 1.00 | 0.00 | 0.053 | 0.00 | 0.00 |
| ATOM | 3769 | HD1 | LEU | 336 | 6.494 | 3.613 | 16.216 | 1.00 | 0.00 | 0.053 | 0.00 | 0.00 |
| ATOM | 3770 | CD2 | LEU | 336 | 7.781 | 1.735 | 14.723 | 1.00 | 0.00 | −0.159 | 16.15 | 4.00 |
| ATOM | 3771 | HD2 | LEU | 336 | 6.901 | 1.267 | 14.280 | 1.00 | 0.00 | 0.053 | 0.00 | 0.00 |
| ATOM | 3772 | HD2 | LEU | 336 | 7.889 | 2.746 | 14.332 | 1.00 | 0.00 | 0.053 | 0.00 | 0.00 |
| ATOM | 3773 | HD2 | LEU | 336 | 8.667 | 1.151 | 14.472 | 1.00 | 0.00 | 0.053 | 0.00 | 0.00 |
| ATOM | 3774 | C | LEU | 336 | 8.790 | 1.202 | 19.048 | 1.00 | 0.00 | 0.396 | 9.82 | 4.00 |
| ATOM | 3775 | O | LEU | 336 | 9.768 | 0.445 | 18.954 | 1.00 | 0.00 | −0.396 | 8.17 | −17.40 |
| ATOM | 3776 | N | ASN | 337 | 7.674 | 0.845 | 19.676 | 1.00 | 0.00 | −0.650 | 9.00 | −17.40 |
| ATOM | 3777 | HN | ASN | 337 | 6.899 | 1.517 | 19.771 | 1.00 | 0.00 | 0.440 | 0.00 | 0.00 |
| ATOM | 3778 | CA | ASN | 337 | 7.546 | −0.494 | 20.228 | 1.00 | 0.00 | 0.158 | 9.40 | 4.00 |
| ATOM | 3779 | HA | ASN | 337 | 8.322 | −0.676 | 20.970 | 1.00 | 0.00 | 0.053 | 0.00 | 0.00 |
| ATOM | 3780 | CB | ASN | 337 | 6.190 | −0.674 | 20.913 | 1.00 | 0.00 | −0.106 | 12.77 | 4.00 |
| ATOM | 3781 | HB1 | ASN | 337 | 5.362 | −0.540 | 20.216 | 1.00 | 0.00 | 0.053 | 0.00 | 0.00 |
| ATOM | 3782 | HB2 | ASN | 337 | 6.044 | 0.043 | 21.720 | 1.00 | 0.00 | 0.053 | 0.00 | 0.00 |
| ATOM | 3783 | CG | ASN | 337 | 6.043 | −2.045 | 21.512 | 1.00 | 0.00 | 0.396 | 9.82 | 4.00 |
| ATOM | 3784 | OD1 | ASN | 337 | 6.814 | −2.433 | 22.392 | 1.00 | 0.00 | −0.396 | 8.17 | −17.40 |
| ATOM | 3785 | ND2 | ASN | 337 | 5.064 | −2.798 | 21.040 | 1.00 | 0.00 | −0.879 | 13.25 | −17.40 |
| ATOM | 3786 | HD2 | ASN | 337 | 4.442 | −2.433 | 20.304 | 1.00 | 0.00 | 0.440 | 0.00 | 0.00 |
| ATOM | 3787 | HD2 | ASN | 337 | 4.922 | −3.749 | 21.408 | 1.00 | 0.00 | 0.440 | 0.00 | 0.00 |
| ATOM | 3788 | C | ASN | 337 | 7.677 | −1.546 | 19.121 | 1.00 | 0.00 | 0.396 | 9.82 | 4.00 |
| ATOM | 3789 | O | ASN | 337 | 7.123 | −1.387 | 18.026 | 1.00 | 0.00 | −0.396 | 8.17 | −17.40 |
| ATOM | 3790 | N | GLN | 338 | 8.401 | −2.622 | 19.417 | 1.00 | 0.00 | −0.650 | 9.00 | −17.40 |
| ATOM | 3791 | HN | GLN | 338 | 8.827 | −2.695 | 20.351 | 1.00 | 0.00 | 0.440 | 0.00 | 0.00 |
| ATOM | 3792 | CA | GLN | 338 | 8.609 | −3.700 | 18.458 | 1.00 | 0.00 | 0.158 | 9.40 | 4.00 |
| ATOM | 3793 | HA | GLN | 338 | 8.149 | −3.497 | 17.490 | 1.00 | 0.00 | 0.053 | 0.00 | 0.00 |
| ATOM | 3794 | CB | GLN | 338 | 10.105 | −3.885 | 18.188 | 1.00 | 0.00 | −0.106 | 12.77 | 4.00 |
| ATOM | 3795 | HB1 | GLN | 338 | 10.230 | −4.740 | 17.524 | 1.00 | 0.00 | 0.053 | 0.00 | 0.00 |
| ATOM | 3796 | HB2 | GLN | 338 | 10.606 | −4.061 | 19.139 | 1.00 | 0.00 | 0.053 | 0.00 | 0.00 |
| ATOM | 3797 | CG | GLN | 338 | 10.774 | −2.681 | 17.526 | 1.00 | 0.00 | −0.106 | 12.77 | 4.00 |
| ATOM | 3798 | HG1 | GLN | 338 | 11.845 | −2.822 | 17.383 | 1.00 | 0.00 | 0.053 | 0.00 | 0.00 |
| ATOM | 3799 | HG2 | GLN | 338 | 10.664 | −1.767 | 18.110 | 1.00 | 0.00 | 0.053 | 0.00 | 0.00 |
| ATOM | 3800 | CD | GLN | 338 | 10.200 | −2.376 | 16.150 | 1.00 | 0.00 | 0.396 | 9.82 | 4.00 |
| ATOM | 3801 | OE1 | GLN | 338 | 10.590 | −1.397 | 15.503 | 1.00 | 0.00 | −0.396 | 8.17 | −17.40 |
| ATOM | 3802 | NE2 | GLN | 338 | 9.272 | −3.215 | 15.694 | 1.00 | 0.00 | −0.879 | 13.25 | −17.40 |
| ATOM | 3803 | HE2 | GLN | 338 | 8.977 | −4.018 | 16.267 | 1.00 | 0.00 | 0.440 | 0.00 | 0.00 |
| ATOM | 3804 | HE2 | GLN | 338 | 8.848 | −3.060 | 14.767 | 1.00 | 0.00 | 0.440 | 0.00 | 0.00 |
| ATOM | 3805 | C | GLN | 338 | 8.028 | −5.029 | 18.931 | 1.00 | 0.00 | 0.396 | 9.82 | 4.00 |
| ATOM | 3806 | O | GLN | 338 | 7.795 | −5.922 | 18.128 | 1.00 | 0.00 | −0.396 | 8.17 | −17.40 |
| ATOM | 3807 | N | GLN | 339 | 7.805 | −5.166 | 20.235 | 1.00 | 0.00 | −0.650 | 9.00 | −17.40 |
| ATOM | 3808 | HN | GLN | 339 | 8.011 | −4.385 | 20.874 | 1.00 | 0.00 | 0.440 | 0.00 | 0.00 |
| ATOM | 3809 | CA | GLN | 339 | 7.269 | −6.417 | 20.766 | 1.00 | 0.00 | 0.158 | 9.40 | 4.00 |
| ATOM | 3810 | HA | GLN | 339 | 7.557 | −7.250 | 20.125 | 1.00 | 0.00 | 0.053 | 0.00 | 0.00 |
| ATOM | 3811 | CB | GLN | 339 | 7.841 | −6.712 | 22.158 | 1.00 | 0.00 | −0.106 | 12.77 | 4.00 |
| ATOM | 3812 | HB1 | GLN | 339 | 7.588 | −5.934 | 22.879 | 1.00 | 0.00 | 0.053 | 0.00 | 0.00 |
| ATOM | 3813 | HB2 | GLN | 339 | 8.928 | −6.787 | 22.148 | 1.00 | 0.00 | 0.053 | 0.00 | 0.00 |
| ATOM | 3814 | CG | GLN | 339 | 7.316 | −8.026 | 22.729 | 1.00 | 0.00 | −0.106 | 12.77 | 4.00 |
| ATOM | 3815 | HG1 | GLN | 339 | 7.538 | −8.895 | 22.110 | 1.00 | 0.00 | 0.053 | 0.00 | 0.00 |
| ATOM | 3816 | HG2 | GLN | 339 | 6.234 | −8.047 | 22.860 | 1.00 | 0.00 | 0.053 | 0.00 | 0.00 |
| ATOM | 3817 | CD | GLN | 339 | 7.880 | −8.369 | 24.096 | 1.00 | 0.00 | 0.396 | 9.82 | 4.00 |
| ATOM | 3818 | OE1 | GLN | 339 | 7.400 | −9.302 | 24.753 | 1.00 | 0.00 | −0.396 | 8.17 | −17.40 |
| ATOM | 3819 | NE2 | GLN | 339 | 8.903 | −7.628 | 24.533 | 1.00 | 0.00 | −0.879 | 13.25 | −17.40 |
| ATOM | 3820 | HE2 | GLN | 339 | 9.269 | −6.861 | 23.950 | 1.00 | 0.00 | 0.440 | 0.00 | 0.00 |
| ATOM | 3821 | HE2 | GLN | 339 | 9.325 | −7.823 | 25.452 | 1.00 | 0.00 | 0.440 | 0.00 | 0.00 |
| ATOM | 3822 | C | GLN | 339 | 5.749 | −6.350 | 20.830 | 1.00 | 0.00 | 0.396 | 9.82 | 4.00 |
| ATOM | 3823 | O | GLN | 339 | 5.180 | −5.503 | 21.522 | 1.00 | 0.00 | −0.396 | 8.17 | −17.40 |
| ATOM | 3824 | N | PHE | 340 | 5.105 | −7.238 | 20.079 | 1.00 | 0.00 | −0.650 | 9.00 | −17.40 |
| ATOM | 3825 | HN | PHE | 340 | 5.649 | −7.918 | 19.529 | 1.00 | 0.00 | 0.440 | 0.00 | 0.00 |
| ATOM | 3826 | CA | PHE | 340 | 3.651 | −7.275 | 20.013 | 1.00 | 0.00 | 0.158 | 9.40 | 4.00 |
| ATOM | 3827 | HA | PHE | 340 | 3.230 | −6.304 | 19.749 | 1.00 | 0.00 | 0.053 | 0.00 | 0.00 |
| ATOM | 3828 | CB | PHE | 340 | 3.183 | −8.262 | 18.931 | 1.00 | 0.00 | −0.106 | 12.77 | 4.00 |
| ATOM | 3829 | HB1 | PHE | 340 | 3.428 | −9.299 | 19.158 | 1.00 | 0.00 | 0.053 | 0.00 | 0.00 |
| ATOM | 3830 | HB2 | PHE | 340 | 3.622 | −8.069 | 17.952 | 1.00 | 0.00 | 0.053 | 0.00 | 0.00 |
| ATOM | 3831 | CG | PHE | 340 | 1.698 | −8.243 | 18.712 | 1.00 | 0.00 | 0.000 | 7.26 | 0.60 |
| ATOM | 3832 | CD1 | PHE | 340 | 1.146 | −7.481 | 17.688 | 1.00 | 0.00 | −0.127 | 10.80 | 0.60 |
| ATOM | 3833 | HD1 | PHE | 340 | 1.804 | −6.950 | 17.000 | 1.00 | 0.00 | 0.127 | 0.00 | 0.00 |
| ATOM | 3834 | CD2 | PHE | 340 | 0.847 | −8.923 | 19.579 | 1.00 | 0.00 | −0.127 | 10.80 | 0.60 |
| ATOM | 3835 | HD2 | PHE | 340 | 1.266 | −9.530 | 20.381 | 1.00 | 0.00 | 0.127 | 0.00 | 0.00 |
| ATOM | 3836 | CE1 | PHE | 340 | −0.233 | −7.385 | 17.527 | 1.00 | 0.00 | −0.127 | 10.80 | 0.60 |
| ATOM | 3837 | HE1 | PHE | 340 | −0.649 | −6.783 | 16.719 | 1.00 | 0.00 | 0.127 | 0.00 | 0.00 |
| ATOM | 3838 | CE2 | PHE | 340 | −0.542 | −8.834 | 19.431 | 1.00 | 0.00 | −0.127 | 10.80 | 0.60 |
| ATOM | 3839 | HE2 | PHE | 340 | −1.200 | −9.367 | 20.117 | 1.00 | 0.00 | 0.127 | 0.00 | 0.00 |
| ATOM | 3840 | CZ | PHE | 340 | −1.080 | −8.063 | 18.406 | 1.00 | 0.00 | −0.127 | 10.80 | 0.60 |
| ATOM | 3841 | HZ | PHE | 340 | −2.161 | −7.988 | 18.289 | 1.00 | 0.00 | 0.127 | 0.00 | 0.00 |
| ATOM | 3842 | C | PHE | 340 | 3.030 | −7.680 | 21.347 | 1.00 | 0.00 | 0.396 | 9.82 | 4.00 |
| ATOM | 3843 | O | PHE | 340 | 3.331 | −8.741 | 21.878 | 1.00 | 0.00 | −0.396 | 8.17 | −17.40 |
| ATOM | 3844 | N | VAL | 341 | 2.166 | −6.827 | 21.883 | 1.00 | 0.00 | −0.650 | 9.00 | −17.40 |
| ATOM | 3845 | HN | VAL | 341 | 1.971 | −5.936 | 21.404 | 1.00 | 0.00 | 0.440 | 0.00 | 0.00 |
| ATOM | 3846 | CA | VAL | 341 | 1.487 | −7.129 | 23.138 | 1.00 | 0.00 | 0.158 | 9.40 | 4.00 |
| ATOM | 3847 | HA | VAL | 341 | 1.967 | −8.017 | 23.549 | 1.00 | 0.00 | 0.053 | 0.00 | 0.00 |
| ATOM | 3848 | CB | VAL | 341 | 1.576 | −5.947 | 24.116 | 1.00 | 0.00 | −0.053 | 9.40 | 4.00 |
| ATOM | 3849 | HB | VAL | 341 | 1.211 | −5.041 | 23.631 | 1.00 | 0.00 | 0.053 | 0.00 | 0.00 |
| ATOM | 3850 | CG1 | VAL | 341 | 0.729 | −6.234 | 25.345 | 1.00 | 0.00 | −0.159 | 16.15 | 4.00 |
| ATOM | 3851 | HG1 | VAL | 341 | 0.794 | −5.393 | 26.036 | 1.00 | 0.00 | 0.053 | 0.00 | 0.00 |
| ATOM | 3852 | HG1 | VAL | 341 | −0.309 | −6.377 | 25.046 | 1.00 | 0.00 | 0.053 | 0.00 | 0.00 |
| ATOM | 3853 | HG1 | VAL | 341 | 1.093 | −7.136 | 25.835 | 1.00 | 0.00 | 0.053 | 0.00 | 0.00 |
| ATOM | 3854 | CG2 | VAL | 341 | 3.035 | −5.703 | 24.518 | 1.00 | 0.00 | −0.159 | 16.15 | 4.00 |
| ATOM | 3855 | HG2 | VAL | 341 | 3.085 | −4.863 | 25.211 | 1.00 | 0.00 | 0.053 | 0.00 | 0.00 |
| ATOM | 3856 | HG2 | VAL | 341 | 3.433 | −6.595 | 24.999 | 1.00 | 0.00 | 0.053 | 0.00 | 0.00 |
| ATOM | 3857 | HG2 | VAL | 341 | 3.624 | −5.476 | 23.629 | 1.00 | 0.00 | 0.053 | 0.00 | 0.00 |
| ATOM | 3858 | C | VAL | 341 | 0.012 | −7.378 | 22.794 | 1.00 | 0.00 | 0.396 | 9.82 | 4.00 |
| ATOM | 3859 | O | VAL | 341 | −0.620 | −6.554 | 22.117 | 1.00 | 0.00 | −0.396 | 8.17 | −17.40 |
| ATOM | 3860 | N | PRO | 342 | −0.547 | −8.521 | 23.228 | 1.00 | 0.00 | −0.422 | 9.00 | −17.40 |
| ATOM | 3861 | CD | PRO | 342 | 0.078 | −9.643 | 23.967 | 1.00 | 0.00 | 0.105 | 12.77 | 4.00 |
| ATOM | 3862 | HD1 | PRO | 342 | 0.559 | −9.293 | 24.880 | 1.00 | 0.00 | 0.053 | 0.00 | 0.00 |
| ATOM | 3863 | HD2 | PRO | 342 | 0.836 | −10.139 | 23.361 | 1.00 | 0.00 | 0.053 | 0.00 | 0.00 |
| ATOM | 3864 | CA | PRO | 342 | −1.958 | −8.806 | 22.925 | 1.00 | 0.00 | 0.158 | 9.40 | 4.00 |
| ATOM | 3865 | HA | PRO | 342 | −2.200 | −8.489 | 21.910 | 1.00 | 0.00 | 0.053 | 0.00 | 0.00 |
| ATOM | 3866 | CB | PRO | 342 | −2.044 | −10.329 | 23.092 | 1.00 | 0.00 | −0.106 | 12.77 | 4.00 |
| ATOM | 3867 | HB1 | PRO | 342 | −1.705 | −10.845 | 22.193 | 1.00 | 0.00 | 0.053 | 0.00 | 0.00 |
| ATOM | 3868 | HB2 | PRO | 342 | −3.067 | −10.648 | 23.290 | 1.00 | 0.00 | 0.053 | 0.00 | 0.00 |
| ATOM | 3869 | CG | PRO | 342 | −1.135 | −10.575 | 24.265 | 1.00 | 0.00 | −0.106 | 12.77 | 4.00 |
| ATOM | 3870 | HG1 | PRO | 342 | −0.839 | −11.622 | 24.320 | 1.00 | 0.00 | 0.053 | 0.00 | 0.00 |
| ATOM | 3871 | HG2 | PRO | 342 | −1.625 | −10.317 | 25.203 | 1.00 | 0.00 | 0.053 | 0.00 | 0.00 |
| ATOM | 3872 | C | PRO | 342 | −2.878 | −8.058 | 23.902 | 1.00 | 0.00 | 0.396 | 9.82 | 4.00 |
| ATOM | 3873 | O | PRO | 342 | −3.560 | −8.677 | 24.725 | 1.00 | 0.00 | −0.396 | 8.17 | −17.40 |
| ATOM | 3874 | N | PHE | 343 | −2.892 | −6.730 | 23.809 | 1.00 | 0.00 | −0.650 | 9.00 | −17.40 |
| ATOM | 3875 | HN | PHE | 343 | −2.307 | −6.266 | 23.098 | 1.00 | 0.00 | 0.440 | 0.00 | 0.00 |
| ATOM | 3876 | CA | PHE | 343 | −3.722 | −5.917 | 24.697 | 1.00 | 0.00 | 0.158 | 9.40 | 4.00 |
| ATOM | 3877 | HA | PHE | 343 | −3.292 | −5.953 | 25.698 | 1.00 | 0.00 | 0.053 | 0.00 | 0.00 |
| ATOM | 3878 | CB | PHE | 343 | −3.718 | −4.443 | 24.283 | 1.00 | 0.00 | −0.106 | 12.77 | 4.00 |
| ATOM | 3879 | HB1 | PHE | 343 | −4.465 | −3.918 | 24.878 | 1.00 | 0.00 | 0.053 | 0.00 | 0.00 |
| ATOM | 3880 | HB2 | PHE | 343 | −3.962 | −4.384 | 23.222 | 1.00 | 0.00 | 0.053 | 0.00 | 0.00 |
| ATOM | 3881 | CG | PHE | 343 | −2.392 | −3.743 | 24.489 | 1.00 | 0.00 | 0.000 | 7.26 | 0.60 |
| ATOM | 3882 | CD1 | PHE | 343 | −1.545 | −3.501 | 23.414 | 1.00 | 0.00 | −0.127 | 10.80 | 0.60 |
| ATOM | 3883 | HD1 | PHE | 343 | −1.801 | −3.890 | 22.428 | 1.00 | 0.00 | 0.127 | 0.00 | 0.00 |
| ATOM | 3884 | CD2 | PHE | 343 | −2.042 | −3.250 | 25.740 | 1.00 | 0.00 | −0.127 | 10.80 | 0.60 |
| ATOM | 3885 | HD2 | PHE | 343 | −2.692 | −3.440 | 26.593 | 1.00 | 0.00 | 0.127 | 0.00 | 0.00 |
| ATOM | 3886 | CE1 | PHE | 343 | −0.371 | −2.766 | 23.581 | 1.00 | 0.00 | −0.127 | 10.80 | 0.60 |
| ATOM | 3887 | HE1 | PHE | 343 | 0.283 | −2.579 | 22.729 | 1.00 | 0.00 | 0.127 | 0.00 | 0.00 |
| ATOM | 3888 | CE2 | PHE | 343 | −0.874 | −2.517 | 25.919 | 1.00 | 0.00 | −0.127 | 10.80 | 0.60 |
| ATOM | 3889 | HE2 | PHE | 343 | −0.615 | −2.135 | 26.907 | 1.00 | 0.00 | 0.127 | 0.00 | 0.00 |
| ATOM | 3890 | CZ | PHE | 343 | −0.038 | −2.271 | 24.839 | 1.00 | 0.00 | −0.127 | 10.80 | 0.60 |
| ATOM | 3891 | HZ | PHE | 343 | 0.875 | −1.692 | 24.975 | 1.00 | 0.00 | 0.127 | 0.00 | 0.00 |
| ATOM | 3892 | C | PHE | 343 | −5.157 | −6.382 | 24.794 | 1.00 | 0.00 | 0.396 | 9.82 | 4.00 |
| ATOM | 3893 | O | PHE | 343 | −5.749 | −6.270 | 25.866 | 1.00 | 0.00 | −0.396 | 8.17 | −17.40 |
| ATOM | 3894 | N | THR | 344 | −5.731 | −6.889 | 23.699 | 1.00 | 0.00 | −0.650 | 9.00 | −17.40 |
| ATOM | 3895 | HN | THR | 344 | −5.205 | −6.943 | 22.815 | 1.00 | 0.00 | 0.440 | 0.00 | 0.00 |
| ATOM | 3896 | CA | THR | 344 | −7.123 | −7.370 | 23.767 | 1.00 | 0.00 | 0.158 | 9.40 | 4.00 |
| ATOM | 3897 | HA | THR | 344 | −7.815 | −6.608 | 24.125 | 1.00 | 0.00 | 0.053 | 0.00 | 0.00 |
| ATOM | 3898 | CB | THR | 344 | −7.717 | −7.720 | 22.359 | 1.00 | 0.00 | 0.060 | 9.40 | 4.00 |
| ATOM | 3899 | HB | THR | 344 | −8.760 | −8.020 | 22.450 | 1.00 | 0.00 | 0.053 | 0.00 | 0.00 |
| ATOM | 3900 | OG1 | THR | 344 | −7.002 | −8.809 | 21.770 | 1.00 | 0.00 | −0.537 | 11.04 | −17.40 |
| ATOM | 3901 | HG1 | THR | 344 | −7.569 | −9.226 | 21.019 | 1.00 | 0.00 | 0.424 | 0.00 | 0.00 |
| ATOM | 3902 | CG2 | THR | 344 | −7.641 | −6.508 | 21.432 | 1.00 | 0.00 | −0.159 | 16.15 | 4.00 |
| ATOM | 3903 | HG2 | THR | 344 | −8.057 | −6.767 | 20.458 | 1.00 | 0.00 | 0.053 | 0.00 | 0.00 |
| ATOM | 3904 | HG2 | THR | 344 | −8.210 | −5.684 | 21.862 | 1.00 | 0.00 | 0.053 | 0.00 | 0.00 |
| ATOM | 3905 | HG2 | THR | 344 | −6.600 | −6.205 | 21.312 | 1.00 | 0.00 | 0.053 | 0.00 | 0.00 |
| ATOM | 3906 | C | THR | 344 | −7.292 | −8.574 | 24.710 | 1.00 | 0.00 | 0.396 | 9.82 | 4.00 |
| ATOM | 3907 | O | THR | 344 | −8.417 | −8.904 | 25.103 | 1.00 | 0.00 | −0.396 | 8.17 | −17.40 |
| ATOM | 3908 | N | GLN | 345 | −6.187 | −9.212 | 25.101 | 1.00 | 0.00 | −0.650 | 9.00 | −17.40 |
| ATOM | 3909 | HN | GLN | 345 | −5.269 | −8.899 | 24.753 | 1.00 | 0.00 | 0.440 | 0.00 | 0.00 |
| ATOM | 3910 | CA | GLN | 345 | −6.262 | −10.356 | 26.022 | 1.00 | 0.00 | 0.158 | 9.40 | 4.00 |
| ATOM | 3911 | HA | GLN | 345 | −7.256 | −10.801 | 26.051 | 1.00 | 0.00 | 0.053 | 0.00 | 0.00 |
| ATOM | 3912 | CB | GLN | 345 | −5.293 | −11.469 | 25.600 | 1.00 | 0.00 | −0.106 | 12.77 | 4.00 |
| ATOM | 3913 | HB1 | GLN | 345 | −5.374 | −12.273 | 26.331 | 1.00 | 0.00 | 0.053 | 0.00 | 0.00 |
| ATOM | 3914 | HB2 | GLN | 345 | −4.288 | −11.045 | 25.589 | 1.00 | 0.00 | 0.053 | 0.00 | 0.00 |
| ATOM | 3915 | CG | GLN | 345 | −5.522 | −12.109 | 24.207 | 1.00 | 0.00 | −0.106 | 12.77 | 4.00 |
| ATOM | 3916 | HG1 | GLN | 345 | −5.513 | −11.358 | 23.416 | 1.00 | 0.00 | 0.053 | 0.00 | 0.00 |
| ATOM | 3917 | HG2 | GLN | 345 | −6.481 | −12.623 | 24.157 | 1.00 | 0.00 | 0.053 | 0.00 | 0.00 |
| ATOM | 3918 | CD | GLN | 345 | −4.432 | −13.137 | 23.875 | 1.00 | 0.00 | 0.396 | 9.82 | 4.00 |
| ATOM | 3919 | OE1 | GLN | 345 | −3.378 | −13.155 | 24.511 | 1.00 | 0.00 | −0.396 | 8.17 | −17.40 |
| ATOM | 3920 | NE2 | GLN | 345 | −4.680 | −13.984 | 22.867 | 1.00 | 0.00 | −0.879 | 13.25 | −17.40 |
| ATOM | 3921 | HE2 | GLN | 345 | −5.575 | −13.934 | 22.361 | 1.00 | 0.00 | 0.440 | 0.00 | 0.00 |
| ATOM | 3922 | HE2 | GLN | 345 | −3.974 | −14.684 | 22.598 | 1.00 | 0.00 | 0.440 | 0.00 | 0.00 |
| ATOM | 3923 | C | GLN | 345 | −5.917 | −9.965 | 27.476 | 1.00 | 0.00 | 0.396 | 9.82 | 4.00 |
| ATOM | 3924 | O | GLN | 345 | −5.904 | −10.814 | 28.360 | 1.00 | 0.00 | −0.396 | 8.17 | −17.40 |
| ATOM | 3925 | N | LEU | 346 | −5.628 | −8.694 | 27.725 | 1.00 | 0.00 | −0.650 | 9.00 | −17.40 |
| ATOM | 3926 | HN | LEU | 346 | −5.670 | −8.009 | 26.957 | 1.00 | 0.00 | 0.440 | 0.00 | 0.00 |
| ATOM | 3927 | CA | LEU | 346 | −5.248 | −8.240 | 29.072 | 1.00 | 0.00 | 0.158 | 9.40 | 4.00 |
| ATOM | 3928 | HA | LEU | 346 | −4.931 | −9.108 | 29.650 | 1.00 | 0.00 | 0.053 | 0.00 | 0.00 |
| ATOM | 3929 | CB | LEU | 346 | −4.096 | −7.244 | 28.965 | 1.00 | 0.00 | −0.106 | 12.77 | 4.00 |
| ATOM | 3930 | HB1 | LEU | 346 | −3.621 | −7.019 | 29.920 | 1.00 | 0.00 | 0.053 | 0.00 | 0.00 |
| ATOM | 3931 | HB2 | LEU | 346 | −4.390 | −6.274 | 28.562 | 1.00 | 0.00 | 0.053 | 0.00 | 0.00 |
| ATOM | 3932 | CG | LEU | 346 | −2.958 | −7.716 | 28.061 | 1.00 | 0.00 | −0.053 | 9.40 | 4.00 |
| ATOM | 3933 | HG | LEU | 346 | −3.326 | −7.899 | 27.051 | 1.00 | 0.00 | 0.053 | 0.00 | 0.00 |
| ATOM | 3934 | CD1 | LEU | 346 | −1.855 | −6.653 | 27.992 | 1.00 | 0.00 | −0.159 | 16.15 | 4.00 |
| ATOM | 3935 | HD1 | LEU | 346 | −1.051 | −7.003 | 27.344 | 1.00 | 0.00 | 0.053 | 0.00 | 0.00 |
| ATOM | 3936 | HD1 | LEU | 346 | −2.266 | −5.727 | 27.590 | 1.00 | 0.00 | 0.053 | 0.00 | 0.00 |
| ATOM | 3937 | HD1 | LEU | 346 | −1.461 | −6.471 | 28.992 | 1.00 | 0.00 | 0.053 | 0.00 | 0.00 |
| ATOM | 3938 | CD2 | LEU | 346 | −2.405 | −9.008 | 28.618 | 1.00 | 0.00 | −0.159 | 16.15 | 4.00 |
| ATOM | 3939 | HD2 | LEU | 346 | −1.591 | −9.358 | 27.983 | 1.00 | 0.00 | 0.053 | 0.00 | 0.00 |
| ATOM | 3940 | HD2 | LEU | 346 | −2.030 | −8.838 | 29.627 | 1.00 | 0.00 | 0.053 | 0.00 | 0.00 |
| ATOM | 3941 | HD2 | LEU | 346 | −3.193 | −9.759 | 28.645 | 1.00 | 0.00 | 0.053 | 0.00 | 0.00 |
| ATOM | 3942 | C | LEU | 346 | −6.368 | −7.560 | 29.834 | 1.00 | 0.00 | 0.396 | 9.82 | 4.00 |
| ATOM | 3943 | O | LEU | 346 | −7.310 | −7.048 | 29.235 | 1.00 | 0.00 | −0.396 | 8.17 | −17.40 |
| ATOM | 3944 | N | ASP | 347P | −6.243 | −7.505 | 31.157 | 1.00 | 0.00 | −0.650 | 9.00 | −17.40 |
| ATOM | 3945 | HN | ASP | 347P | −5.424 | −7.936 | 31.610 | 1.00 | 0.00 | 0.440 | 0.00 | 0.00 |
| ATOM | 3946 | CA | ASP | 347P | −7.252 | −6.841 | 31.971 | 1.00 | 0.00 | 0.158 | 9.40 | 4.00 |
| ATOM | 3947 | HA | ASP | 347P | −8.193 | −6.923 | 31.427 | 1.00 | 0.00 | 0.053 | 0.00 | 0.00 |
| ATOM | 3948 | CB | ASP | 347P | −7.352 | −7.510 | 33.346 | 1.00 | 0.00 | −0.336 | 12.77 | 4.00 |
| ATOM | 3949 | HB1 | ASP | 347P | −6.427 | −7.435 | 33.919 | 1.00 | 0.00 | 0.053 | 0.00 | 0.00 |
| ATOM | 3950 | HB2 | ASP | 347P | −7.580 | −8.573 | 33.281 | 1.00 | 0.00 | 0.053 | 0.00 | 0.00 |
| ATOM | 3951 | CG | ASP | 347P | −8.439 | −6.899 | 34.220 | 1.00 | 0.00 | 0.297 | 9.82 | 4.00 |
| ATOM | 3952 | OD1 | ASP | 347P | −8.914 | −5.785 | 33.900 | 1.00 | 0.00 | −0.534 | 8.17 | −18.95 |
| ATOM | 3953 | OD2 | ASP | 347P | −8.814 | −7.542 | 35.238 | 1.00 | 0.00 | −0.534 | 8.17 | −18.95 |
| ATOM | 3954 | C | ASP | 347P | −6.797 | −5.382 | 32.128 | 1.00 | 0.00 | 0.396 | 9.82 | 4.00 |
| ATOM | 3955 | O | ASP | 347P | −5.851 | −5.100 | 32.859 | 1.00 | 0.00 | −0.396 | 8.17 | −17.40 |
| ATOM | 3956 | N | LEU | 348 | −7.446 | −4.456 | 31.425 | 1.00 | 0.00 | −0.650 | 9.00 | −17.40 |
| ATOM | 3957 | HN | LEU | 348 | −8.226 | −4.733 | 30.813 | 1.00 | 0.00 | 0.440 | 0.00 | 0.00 |
| ATOM | 3958 | CA | LEU | 348 | −7.062 | −3.056 | 31.513 | 1.00 | 0.00 | 0.158 | 9.40 | 4.00 |
| ATOM | 3959 | HA | LEU | 348 | −6.044 | −2.915 | 31.878 | 1.00 | 0.00 | 0.053 | 0.00 | 0.00 |
| ATOM | 3960 | CB | LEU | 348 | −7.092 | −2.417 | 30.112 | 1.00 | 0.00 | −0.106 | 12.77 | 4.00 |
| ATOM | 3961 | HB1 | LEU | 348 | −6.747 | −1.383 | 30.099 | 1.00 | 0.00 | 0.053 | 0.00 | 0.00 |
| ATOM | 3962 | HB2 | LEU | 348 | −8.087 | −2.392 | 29.668 | 1.00 | 0.00 | 0.053 | 0.00 | 0.00 |
| ATOM | 3963 | CG | LEU | 348 | −6.212 | −3.148 | 29.089 | 1.00 | 0.00 | −0.053 | 9.40 | 4.00 |
| ATOM | 3964 | HG | LEU | 348 | −6.559 | −4.172 | 28.955 | 1.00 | 0.00 | 0.053 | 0.00 | 0.00 |
| ATOM | 3965 | CD1 | LEU | 348 | −6.265 | −2.421 | 27.714 | 1.00 | 0.00 | −0.159 | 16.15 | 4.00 |
| ATOM | 3966 | HD1 | LEU | 348 | −5.636 | −2.950 | 26.998 | 1.00 | 0.00 | 0.053 | 0.00 | 0.00 |
| ATOM | 3967 | HD1 | LEU | 348 | −7.292 | −2.403 | 27.351 | 1.00 | 0.00 | 0.053 | 0.00 | 0.00 |
| ATOM | 3968 | HD1 | LEU | 348 | −5.903 | −1.399 | 27.828 | 1.00 | 0.00 | 0.053 | 0.00 | 0.00 |
| ATOM | 3969 | CD2 | LEU | 348 | −4.788 | −3.231 | 29.631 | 1.00 | 0.00 | −0.159 | 16.15 | 4.00 |
| ATOM | 3970 | HD2 | LEU | 348 | −4.154 | −3.749 | 28.911 | 1.00 | 0.00 | 0.053 | 0.00 | 0.00 |
| ATOM | 3971 | HD2 | LEU | 348 | −4.402 | −2.225 | 29.796 | 1.00 | 0.00 | 0.053 | 0.00 | 0.00 |
| ATOM | 3972 | HD2 | LEU | 348 | −4.788 | −3.778 | 30.573 | 1.00 | 0.00 | 0.053 | 0.00 | 0.00 |
| ATOM | 3973 | C | LEU | 348 | −7.954 | −2.250 | 32.454 | 1.00 | 0.00 | 0.396 | 9.82 | 4.00 |
| ATOM | 3974 | O | LEU | 348 | −8.040 | −1.030 | 32.329 | 1.00 | 0.00 | −0.396 | 8.17 | −17.40 |
| ATOM | 3975 | N | SER | 349 | −8.613 | −2.918 | 33.395 | 1.00 | 0.00 | −0.650 | 9.00 | −17.40 |
| ATOM | 3976 | HN | SER | 349 | −8.502 | −3.938 | 33.475 | 1.00 | 0.00 | 0.440 | 0.00 | 0.00 |
| ATOM | 3977 | CA | SER | 349 | −9.497 | −2.202 | 34.319 | 1.00 | 0.00 | 0.158 | 9.40 | 4.00 |
| ATOM | 3978 | HA | SER | 349 | −10.212 | −1.568 | 33.795 | 1.00 | 0.00 | 0.053 | 0.00 | 0.00 |
| ATOM | 3979 | CB | SER | 349 | −10.348 | −3.186 | 35.134 | 1.00 | 0.00 | 0.007 | 12.77 | 4.00 |
| ATOM | 3980 | HB1 | SER | 349 | −10.967 | −3.802 | 34.482 | 1.00 | 0.00 | 0.053 | 0.00 | 0.00 |
| ATOM | 3981 | HB2 | SER | 349 | −11.010 | −2.658 | 35.820 | 1.00 | 0.00 | 0.053 | 0.00 | 0.00 |
| ATOM | 3982 | OG | SER | 349 | −9.534 | −4.050 | 35.900 | 1.00 | 0.00 | −0.537 | 11.04 | −17.40 |
| ATOM | 3983 | HG | SER | 349 | −9.929 | −4.146 | 36.846 | 1.00 | 0.00 | 0.424 | 0.00 | 0.00 |
| ATOM | 3984 | C | SER | 349 | −8.722 | −1.295 | 35.269 | 1.00 | 0.00 | 0.396 | 9.82 | 4.00 |
| ATOM | 3985 | O | SER | 349 | −9.303 | −0.426 | 35.921 | 1.00 | 0.00 | −0.396 | 8.17 | −17.40 |
| ATOM | 3986 | N | TYR | 350 | −7.416 | −1.506 | 35.363 | 1.00 | 0.00 | −0.650 | 9.00 | −17.40 |
| ATOM | 3987 | HN | TYR | 350 | −6.979 | −2.258 | 34.811 | 1.00 | 0.00 | 0.440 | 0.00 | 0.00 |
| ATOM | 3988 | CA | TYR | 350 | −6.591 | −0.680 | 36.243 | 1.00 | 0.00 | 0.158 | 9.40 | 4.00 |
| ATOM | 3989 | HA | TYR | 350 | −6.990 | −0.650 | 37.256 | 1.00 | 0.00 | 0.053 | 0.00 | 0.00 |
| ATOM | 3990 | CB | TYR | 350 | −5.181 | −1.292 | 36.378 | 1.00 | 0.00 | −0.106 | 12.77 | 4.00 |
| ATOM | 3991 | HB1 | TYR | 350 | −5.296 | −2.341 | 36.649 | 1.00 | 0.00 | 0.053 | 0.00 | 0.00 |
| ATOM | 3992 | HB2 | TYR | 350 | −4.650 | −0.743 | 37.156 | 1.00 | 0.00 | 0.053 | 0.00 | 0.00 |
| ATOM | 3993 | CG | TYR | 350 | −4.316 | −1.246 | 35.132 | 1.00 | 0.00 | 0.000 | 7.26 | 0.60 |
| ATOM | 3994 | CD1 | TYR | 350 | −3.343 | −0.252 | 34.962 | 1.00 | 0.00 | −0.127 | 10.80 | 0.60 |
| ATOM | 3995 | HD1 | TYR | 350 | −3.240 | 0.528 | 35.715 | 1.00 | 0.00 | 0.127 | 0.00 | 0.00 |
| ATOM | 3996 | CE1 | TYR | 350 | −2.503 | −0.240 | 33.850 | 1.00 | 0.00 | −0.127 | 10.80 | 0.60 |
| ATOM | 3997 | HE1 | TYR | 350 | −1.747 | 0.538 | 33.744 | 1.00 | 0.00 | 0.127 | 0.00 | 0.00 |
| ATOM | 3998 | CD2 | TYR | 350 | −4.431 | −2.223 | 34.140 | 1.00 | 0.00 | −0.127 | 10.80 | 0.60 |
| ATOM | 3999 | HD2 | TYR | 350 | −5.181 | −3.007 | 34.245 | 1.00 | 0.00 | 0.127 | 0.00 | 0.00 |
| ATOM | 4000 | CE2 | TYR | 350 | −3.594 | −2.213 | 33.005 | 1.00 | 0.00 | −0.127 | 10.80 | 0.60 |
| ATOM | 4001 | HE2 | TYR | 350 | −3.703 | −2.977 | 32.235 | 1.00 | 0.00 | 0.127 | 0.00 | 0.00 |
| ATOM | 4002 | CZ | TYR | 350 | −2.632 | −1.226 | 32.874 | 1.00 | 0.00 | 0.026 | 7.26 | 0.60 |
| ATOM | 4003 | OH | TYR | 350 | −1.776 | −1.247 | 31.788 | 1.00 | 0.00 | −0.451 | 10.94 | −17.40 |
| ATOM | 4004 | HH | TYR | 350 | −1.577 | −0.281 | 31.487 | 1.00 | 0.00 | 0.424 | 0.00 | 0.00 |
| ATOM | 4005 | C | TYR | 350 | −6.508 | 0.773 | 35.743 | 1.00 | 0.00 | 0.396 | 9.82 | 4.00 |
| ATOM | 4006 | O | TYR | 350 | −6.112 | 1.667 | 36.501 | 1.00 | 0.00 | −0.396 | 8.17 | −17.40 |
| ATOM | 4007 | N | LEU | 351 | −6.889 | 1.005 | 34.479 | 1.00 | 0.00 | −0.650 | 9.00 | −17.40 |
| ATOM | 4008 | HN | LEU | 351 | −7.210 | 0.217 | 33.897 | 1.00 | 0.00 | 0.440 | 0.00 | 0.00 |
| ATOM | 4009 | CA | LEU | 351 | −6.859 | 2.347 | 33.909 | 1.00 | 0.00 | 0.158 | 9.40 | 4.00 |
| ATOM | 4010 | HA | LEU | 351 | −6.003 | 2.877 | 34.328 | 1.00 | 0.00 | 0.053 | 0.00 | 0.00 |
| ATOM | 4011 | CB | LEU | 351 | −6.638 | 2.276 | 32.390 | 1.00 | 0.00 | −0.106 | 12.77 | 4.00 |
| ATOM | 4012 | HB1 | LEU | 351 | −6.818 | 3.269 | 31.980 | 1.00 | 0.00 | 0.053 | 0.00 | 0.00 |
| ATOM | 4013 | HB2 | LEU | 351 | −7.343 | 1.550 | 31.984 | 1.00 | 0.00 | 0.053 | 0.00 | 0.00 |
| ATOM | 4014 | CG | LEU | 351 | −5.242 | 1.849 | 31.924 | 1.00 | 0.00 | −0.053 | 9.40 | 4.00 |
| ATOM | 4015 | HG | LEU | 351 | −4.959 | 0.924 | 32.428 | 1.00 | 0.00 | 0.053 | 0.00 | 0.00 |
| ATOM | 4016 | CD1 | LEU | 351 | −5.260 | 1.622 | 30.405 | 1.00 | 0.00 | −0.159 | 16.15 | 4.00 |
| ATOM | 4017 | HD1 | LEU | 351 | −4.268 | 1.318 | 30.070 | 1.00 | 0.00 | 0.053 | 0.00 | 0.00 |
| ATOM | 4018 | HD1 | LEU | 351 | −5.979 | 0.840 | 30.162 | 1.00 | 0.00 | 0.053 | 0.00 | 0.00 |
| ATOM | 4019 | HD1 | LEU | 351 | −5.544 | 2.546 | 29.902 | 1.00 | 0.00 | 0.053 | 0.00 | 0.00 |
| ATOM | 4020 | CD2 | LEU | 351 | −4.216 | 2.940 | 32.294 | 1.00 | 0.00 | −0.159 | 16.15 | 4.00 |
| ATOM | 4021 | HD2 | LEU | 351 | −3.223 | 2.634 | 31.961 | 1.00 | 0.00 | 0.053 | 0.00 | 0.00 |
| ATOM | 4022 | HD2 | LEU | 351 | −4.489 | 3.876 | 31.807 | 1.00 | 0.00 | 0.053 | 0.00 | 0.00 |
| ATOM | 4023 | HD2 | LEU | 351 | −4.209 | 3.081 | 33.374 | 1.00 | 0.00 | 0.053 | 0.00 | 0.00 |
| ATOM | 4024 | C | LEU | 351 | −8.108 | 3.184 | 34.194 | 1.00 | 0.00 | 0.396 | 9.82 | 4.00 |
| ATOM | 4025 | O | LEU | 351 | −8.130 | 4.389 | 33.921 | 1.00 | 0.00 | −0.396 | 8.17 | −17.40 |
| ATOM | 4026 | N | GLN | 352 | −9.156 | 2.555 | 34.709 | 1.00 | 0.00 | −0.650 | 9.00 | −17.40 |
| ATOM | 4027 | HN | GLN | 352 | −9.111 | 1.540 | 34.879 | 1.00 | 0.00 | 0.440 | 0.00 | 0.00 |
| ATOM | 4028 | CA | GLN | 352 | −10.371 | 3.293 | 35.035 | 1.00 | 0.00 | 0.158 | 9.40 | 4.00 |
| ATOM | 4029 | HA | GLN | 352 | −10.659 | 3.851 | 34.144 | 1.00 | 0.00 | 0.053 | 0.00 | 0.00 |
| ATOM | 4030 | CB | GLN | 352 | −11.488 | 2.340 | 35.450 | 1.00 | 0.00 | −0.106 | 12.77 | 4.00 |
| ATOM | 4031 | HB1 | GLN | 352 | −12.335 | 2.935 | 35.790 | 1.00 | 0.00 | 0.053 | 0.00 | 0.00 |
| ATOM | 4032 | HB2 | GLN | 352 | −11.114 | 1.706 | 36.255 | 1.00 | 0.00 | 0.053 | 0.00 | 0.00 |
| ATOM | 4033 | CG | GLN | 352 | −11.983 | 1.429 | 34.342 | 1.00 | 0.00 | −0.106 | 12.77 | 4.00 |
| ATOM | 4034 | HG1 | GLN | 352 | −11.162 | 1.003 | 33.764 | 1.00 | 0.00 | 0.053 | 0.00 | 0.00 |
| ATOM | 4035 | HG2 | GLN | 352 | −12.624 | 1.955 | 33.635 | 1.00 | 0.00 | 0.053 | 0.00 | 0.00 |
| ATOM | 4036 | CD | GLN | 352 | −12.792 | 0.260 | 34.882 | 1.00 | 0.00 | 0.396 | 9.82 | 4.00 |
| ATOM | 4037 | OE1 | GLN | 352 | −13.401 | −0.495 | 34.118 | 1.00 | 0.00 | −0.396 | 8.17 | −17.40 |
| ATOM | 4038 | NE2 | GLN | 352 | −12.798 | 0.100 | 36.206 | 1.00 | 0.00 | −0.879 | 13.25 | −17.40 |
| ATOM | 4039 | HE2 | GLN | 352 | −12.275 | 0.754 | 36.806 | 1.00 | 0.00 | 0.440 | 0.00 | 0.00 |
| ATOM | 4040 | HE2 | GLN | 352 | −13.325 | −0.677 | 36.628 | 1.00 | 0.00 | 0.440 | 0.00 | 0.00 |
| ATOM | 4041 | C | GLN | 352 | −10.039 | 4.222 | 36.195 | 1.00 | 0.00 | 0.396 | 9.82 | 4.00 |
| ATOM | 4042 | O | GLN | 352 | −9.312 | 3.853 | 37.127 | 1.00 | 0.00 | −0.396 | 8.17 | −17.40 |
| ATOM | 4043 | N | GLN | 353 | −10.588 | 5.427 | 36.130 | 1.00 | 0.00 | −0.650 | 9.00 | −17.40 |
| ATOM | 4044 | HN | GLN | 353 | −11.208 | 5.640 | 35.336 | 1.00 | 0.00 | 0.440 | 0.00 | 0.00 |
| ATOM | 4045 | CA | GLN | 353 | −10.356 | 6.462 | 37.126 | 1.00 | 0.00 | 0.158 | 9.40 | 4.00 |
| ATOM | 4046 | HA | GLN | 353 | −9.350 | 6.851 | 36.965 | 1.00 | 0.00 | 0.053 | 0.00 | 0.00 |
| ATOM | 4047 | CB | GLN | 353 | −11.308 | 7.612 | 36.836 | 1.00 | 0.00 | −0.106 | 12.77 | 4.00 |
| ATOM | 4048 | HB1 | GLN | 353 | −12.302 | 7.192 | 36.683 | 1.00 | 0.00 | 0.053 | 0.00 | 0.00 |
| ATOM | 4049 | HB2 | GLN | 353 | −10.955 | 8.119 | 35.938 | 1.00 | 0.00 | 0.053 | 0.00 | 0.00 |
| ATOM | 4050 | CG | GLN | 353 | −11.430 | 8.643 | 37.897 | 1.00 | 0.00 | −0.106 | 12.77 | 4.00 |
| ATOM | 4051 | HG1 | GLN | 353 | −10.457 | 9.078 | 38.127 | 1.00 | 0.00 | 0.053 | 0.00 | 0.00 |
| ATOM | 4052 | HG2 | GLN | 353 | −11.833 | 8.210 | 38.812 | 1.00 | 0.00 | 0.053 | 0.00 | 0.00 |
| ATOM | 4053 | CD | GLN | 353 | −12.354 | 9.761 | 37.458 | 1.00 | 0.00 | 0.396 | 9.82 | 4.00 |
| ATOM | 4054 | OE1 | GLN | 353 | −13.495 | 9.519 | 37.030 | 1.00 | 0.00 | −0.396 | 8.17 | −17.40 |
| ATOM | 4055 | NE2 | GLN | 353 | −11.865 | 10.983 | 37.540 | 1.00 | 0.00 | −0.879 | 13.25 | −17.40 |
| ATOM | 4056 | HE2 | GLN | 353 | −10.912 | 11.133 | 37.902 | 1.00 | 0.00 | 0.440 | 0.00 | 0.00 |
| ATOM | 4057 | HE2 | GLN | 353 | −12.435 | 11.787 | 37.242 | 1.00 | 0.00 | 0.440 | 0.00 | 0.00 |
| ATOM | 4058 | C | GLN | 353 | −10.435 | 6.058 | 38.596 | 1.00 | 0.00 | 0.396 | 9.82 | 4.00 |
| ATOM | 4059 | O | GLN | 353 | −9.514 | 6.349 | 39.367 | 1.00 | 0.00 | −0.396 | 8.17 | −17.40 |
| ATOM | 4060 | N | GLU | 354 | −11.510 | 5.380 | 38.991 | 1.00 | 0.00 | −0.650 | 9.00 | −17.40 |
| ATOM | 4061 | HN | GLU | 354 | −12.235 | 5.125 | 38.306 | 1.00 | 0.00 | 0.440 | 0.00 | 0.00 |
| ATOM | 4062 | CA | GLU | 354 | −11.666 | 4.994 | 40.394 | 1.00 | 0.00 | 0.158 | 9.40 | 4.00 |
| ATOM | 4063 | HA | GLU | 354 | −11.575 | 5.890 | 41.007 | 1.00 | 0.00 | 0.053 | 0.00 | 0.00 |
| ATOM | 4064 | CB | GLU | 354 | −13.090 | 4.474 | 40.681 | 1.00 | 0.00 | −0.106 | 12.77 | 4.00 |
| ATOM | 4065 | HB1 | GLU | 354 | −13.796 | 5.214 | 40.306 | 1.00 | 0.00 | 0.053 | 0.00 | 0.00 |
| ATOM | 4066 | HB2 | GLU | 354 | −13.187 | 4.349 | 41.759 | 1.00 | 0.00 | 0.053 | 0.00 | 0.00 |
| ATOM | 4067 | CG | GLU | 354 | −13.467 | 3.135 | 40.042 | 1.00 | 0.00 | −0.106 | 12.77 | 4.00 |
| ATOM | 4068 | HG1 | GLU | 354 | −14.303 | 2.640 | 40.535 | 1.00 | 0.00 | 0.053 | 0.00 | 0.00 |
| ATOM | 4069 | HG2 | GLU | 354 | −12.655 | 2.407 | 40.052 | 1.00 | 0.00 | 0.053 | 0.00 | 0.00 |
| ATOM | 4070 | CD | GLU | 354 | −13.877 | 3.257 | 38.584 | 1.00 | 0.00 | 0.399 | 9.82 | 4.00 |
| ATOM | 4071 | OE1 | GLU | 354 | −14.440 | 2.275 | 38.044 | 1.00 | 0.00 | −0.396 | 8.17 | −18.95 |
| ATOM | 4072 | OE2 | GLU | 354 | −13.634 | 4.326 | 37.980 | 1.00 | 0.00 | −0.427 | 8.17 | −18.95 |
| ATOM | 4073 | HE2 | GLU | 354 | −13.964 | 4.254 | 37.007 | 1.00 | 0.00 | 0.424 | 0.00 | 0.00 |
| ATOM | 4074 | C | GLU | 354 | −10.634 | 3.991 | 40.891 | 1.00 | 0.00 | 0.396 | 9.82 | 4.00 |
| ATOM | 4075 | O | GLU | 354 | −10.442 | 3.844 | 42.101 | 1.00 | 0.00 | −0.396 | 8.17 | −17.40 |
| ATOM | 4076 | N | ALA | 355 | −9.964 | 3.290 | 39.980 | 1.00 | 0.00 | −0.650 | 9.00 | −17.40 |
| ATOM | 4077 | HN | ALA | 355 | −10.172 | 3.409 | 38.978 | 1.00 | 0.00 | 0.440 | 0.00 | 0.00 |
| ATOM | 4078 | CA | ALA | 355 | −8.929 | 2.348 | 40.408 | 1.00 | 0.00 | 0.158 | 9.40 | 4.00 |
| ATOM | 4079 | HA | ALA | 355 | −9.148 | 2.029 | 41.427 | 1.00 | 0.00 | 0.053 | 0.00 | 0.00 |
| ATOM | 4080 | CB | ALA | 355 | −8.920 | 1.110 | 39.492 | 1.00 | 0.00 | −0.159 | 16.15 | 4.00 |
| ATOM | 4081 | HB1 | ALA | 355 | −8.145 | 0.418 | 39.822 | 1.00 | 0.00 | 0.053 | 0.00 | 0.00 |
| ATOM | 4082 | HB2 | ALA | 355 | −9.890 | 0.616 | 39.538 | 1.00 | 0.00 | 0.053 | 0.00 | 0.00 |
| ATOM | 4083 | HB3 | ALA | 355 | −8.717 | 1.417 | 38.466 | 1.00 | 0.00 | 0.053 | 0.00 | 0.00 |
| ATOM | 4084 | C | ALA | 355 | −7.559 | 3.062 | 40.352 | 1.00 | 0.00 | 0.396 | 9.82 | 4.00 |
| ATOM | 4085 | O | ALA | 355 | −6.799 | 3.077 | 41.330 | 1.00 | 0.00 | −0.396 | 8.17 | −17.40 |
| ATOM | 4086 | N | TYR | 356 | −7.267 | 3.663 | 39.203 | 1.00 | 0.00 | −0.650 | 9.00 | −17.40 |
| ATOM | 4087 | HN | TYR | 356 | −7.956 | 3.632 | 38.438 | 1.00 | 0.00 | 0.440 | 0.00 | 0.00 |
| ATOM | 4088 | CA | TYR | 356 | −6.005 | 4.366 | 38.984 | 1.00 | 0.00 | 0.158 | 9.40 | 4.00 |
| ATOM | 4089 | HA | TYR | 356 | −5.196 | 3.635 | 39.012 | 1.00 | 0.00 | 0.053 | 0.00 | 0.00 |
| ATOM | 4090 | CB | TYR | 356 | −6.007 | 4.999 | 37.588 | 1.00 | 0.00 | −0.106 | 12.77 | 4.00 |
| ATOM | 4091 | HB1 | TYR | 356 | −6.530 | 5.953 | 37.646 | 1.00 | 0.00 | 0.053 | 0.00 | 0.00 |
| ATOM | 4092 | HB2 | TYR | 356 | −6.520 | 4.320 | 36.906 | 1.00 | 0.00 | 0.053 | 0.00 | 0.00 |
| ATOM | 4093 | CG | TYR | 356 | −4.639 | 5.264 | 37.022 | 1.00 | 0.00 | 0.000 | 7.26 | 0.60 |
| ATOM | 4094 | CD1 | TYR | 356 | −3.935 | 4.255 | 36.360 | 1.00 | 0.00 | −0.127 | 10.80 | 0.60 |
| ATOM | 4095 | HD1 | TYR | 356 | −4.379 | 3.263 | 36.270 | 1.00 | 0.00 | 0.127 | 0.00 | 0.00 |
| ATOM | 4096 | CE1 | TYR | 356 | −2.679 | 4.490 | 35.814 | 1.00 | 0.00 | −0.127 | 10.80 | 0.60 |
| ATOM | 4097 | HE1 | TYR | 356 | −2.145 | 3.691 | 35.298 | 1.00 | 0.00 | 0.127 | 0.00 | 0.00 |
| ATOM | 4098 | CD2 | TYR | 356 | −4.045 | 6.525 | 37.130 | 1.00 | 0.00 | −0.127 | 10.80 | 0.60 |
| ATOM | 4099 | HD2 | TYR | 356 | −4.574 | 7.325 | 37.646 | 1.00 | 0.00 | 0.127 | 0.00 | 0.00 |
| ATOM | 4100 | CE2 | TYR | 356 | −2.789 | 6.772 | 36.589 | 1.00 | 0.00 | −0.127 | 10.80 | 0.60 |
| ATOM | 4101 | HE2 | TYR | 356 | −2.336 | 7.759 | 36.677 | 1.00 | 0.00 | 0.127 | 0.00 | 0.00 |
| ATOM | 4102 | CZ | TYR | 356 | −2.113 | 5.743 | 35.931 | 1.00 | 0.00 | 0.026 | 7.26 | 0.60 |
| ATOM | 4103 | OH | TYR | 356 | −0.880 | 5.958 | 35.375 | 1.00 | 0.00 | −0.451 | 10.94 | −17.40 |
| ATOM | 4104 | HH | TYR | 356 | −0.418 | 6.742 | 35.856 | 1.00 | 0.00 | 0.424 | 0.00 | 0.00 |
| ATOM | 4105 | C | TYR | 356 | −5.696 | 5.447 | 40.033 | 1.00 | 0.00 | 0.396 | 9.82 | 4.00 |
| ATOM | 4106 | O | TYR | 356 | −4.605 | 5.459 | 40.598 | 1.00 | 0.00 | −0.396 | 8.17 | −17.40 |
| ATOM | 4107 | N | ASP | 357P | −6.645 | 6.343 | 40.293 | 1.00 | 0.00 | −0.650 | 9.00 | −17.40 |
| ATOM | 4108 | HN | ASP | 357P | −7.550 | 6.277 | 39.805 | 1.00 | 0.00 | 0.440 | 0.00 | 0.00 |
| ATOM | 4109 | CA | ASP | 357P | −6.425 | 7.424 | 41.262 | 1.00 | 0.00 | 0.158 | 9.40 | 4.00 |
| ATOM | 4110 | HA | ASP | 357P | −5.506 | 7.942 | 40.988 | 1.00 | 0.00 | 0.053 | 0.00 | 0.00 |
| ATOM | 4111 | CB | ASP | 357P | −7.538 | 8.476 | 41.144 | 1.00 | 0.00 | −0.336 | 12.77 | 4.00 |
| ATOM | 4112 | HB1 | ASP | 357P | −7.500 | 9.215 | 41.944 | 1.00 | 0.00 | 0.053 | 0.00 | 0.00 |
| ATOM | 4113 | HB2 | ASP | 357P | −8.534 | 8.036 | 41.182 | 1.00 | 0.00 | 0.053 | 0.00 | 0.00 |
| ATOM | 4114 | CG | ASP | 357P | −7.467 | 9.258 | 39.837 | 1.00 | 0.00 | 0.297 | 9.82 | 4.00 |
| ATOM | 4115 | OD1 | ASP | 357P | −6.645 | 8.900 | 38.962 | 1.00 | 0.00 | −0.534 | 8.17 | −18.95 |
| ATOM | 4116 | OD2 | ASP | 357P | −8.236 | 10.235 | 39.677 | 1.00 | 0.00 | −0.534 | 8.17 | −18.95 |
| ATOM | 4117 | C | ASP | 357P | −6.279 | 6.969 | 42.717 | 1.00 | 0.00 | 0.396 | 9.82 | 4.00 |
| ATOM | 4118 | O | ASP | 357P | −5.941 | 7.762 | 43.595 | 1.00 | 0.00 | −0.396 | 8.17 | −17.40 |
| ATOM | 4119 | N | ARG | 358G | −6.540 | 5.699 | 42.983 | 1.00 | 0.00 | −0.650 | 9.00 | −17.40 |
| ATOM | 4120 | HN | ARG | 358G | −6.860 | 5.073 | 42.230 | 1.00 | 0.00 | 0.440 | 0.00 | 0.00 |
| ATOM | 4121 | CA | ARG | 358G | −6.377 | 5.182 | 44.330 | 1.00 | 0.00 | 0.158 | 9.40 | 4.00 |
| ATOM | 4122 | HA | ARG | 358G | −6.354 | 6.044 | 44.996 | 1.00 | 0.00 | 0.053 | 0.00 | 0.00 |
| ATOM | 4123 | CB | ARG | 358G | −7.543 | 4.248 | 44.686 | 1.00 | 0.00 | −0.106 | 12.77 | 4.00 |
| ATOM | 4124 | HB1 | ARG | 358G | −7.564 | 3.433 | 43.962 | 1.00 | 0.00 | 0.053 | 0.00 | 0.00 |
| ATOM | 4125 | HB2 | ARG | 358G | −8.468 | 4.821 | 44.643 | 1.00 | 0.00 | 0.053 | 0.00 | 0.00 |
| ATOM | 4126 | CG | ARG | 358G | −7.441 | 3.637 | 46.068 | 1.00 | 0.00 | −0.106 | 12.77 | 4.00 |
| ATOM | 4127 | HG1 | ARG | 358G | −8.371 | 3.749 | 46.624 | 1.00 | 0.00 | 0.053 | 0.00 | 0.00 |
| ATOM | 4128 | HG2 | ARG | 358G | −6.652 | 4.107 | 46.655 | 1.00 | 0.00 | 0.053 | 0.00 | 0.00 |
| ATOM | 4129 | CD | ARG | 358G | −7.129 | 2.145 | 45.998 | 1.00 | 0.00 | 0.374 | 12.77 | 4.00 |
| ATOM | 4130 | HD1 | ARG | 358G | −6.592 | 1.775 | 46.872 | 1.00 | 0.00 | 0.053 | 0.00 | 0.00 |
| ATOM | 4131 | HD2 | ARG | 358G | −6.510 | 1.873 | 45.142 | 1.00 | 0.00 | 0.053 | 0.00 | 0.00 |
| ATOM | 4132 | NE | ARG | 358G | −8.326 | 1.307 | 45.893 | 1.00 | 0.00 | −0.819 | 9.00 | −24.67 |
| ATOM | 4133 | HE | ARG | 358G | −9.241 | 1.778 | 45.885 | 1.00 | 0.00 | 0.407 | 0.00 | 0.00 |
| ATOM | 4134 | CZ | ARG | 358G | −8.312 | −0.024 | 45.807 | 1.00 | 0.00 | 0.796 | 6.95 | 4.00 |
| ATOM | 4135 | NH1 | ARG | 358G | −7.159 | −0.688 | 45.802 | 1.00 | 0.00 | −0.746 | 9.00 | −24.67 |
| ATOM | 4136 | HH1 | ARG | 358G | −7.156 | −1.715 | 45.735 | 1.00 | 0.00 | 0.407 | 0.00 | 0.00 |
| ATOM | 4137 | HH1 | ARG | 358G | −6.268 | −0.174 | 45.864 | 1.00 | 0.00 | 0.407 | 0.00 | 0.00 |
| ATOM | 4138 | NH2 | ARG | 358G | −9.455 | −0.699 | 45.751 | 1.00 | 0.00 | −0.746 | 9.00 | −24.67 |
| ATOM | 4139 | HH2 | ARG | 358G | −9.443 | −1.726 | 45.684 | 1.00 | 0.00 | 0.407 | 0.00 | 0.00 |
| ATOM | 4140 | HH2 | ARG | 358G | −10.352 | −0.194 | 45.774 | 1.00 | 0.00 | 0.407 | 0.00 | 0.00 |
| ATOM | 4141 | C | ARG | 358G | −5.061 | 4.409 | 44.348 | 1.00 | 0.00 | 0.396 | 9.82 | 4.00 |
| ATOM | 4142 | O | ARG | 358G | −4.143 | 4.715 | 45.126 | 1.00 | 0.00 | −0.396 | 8.17 | −17.40 |
| ATOM | 4143 | N | ASP | 359P | −4.963 | 3.431 | 43.447 | 1.00 | 0.00 | −0.650 | 9.00 | −17.40 |
| ATOM | 4144 | HN | ASP | 359P | −5.744 | 3.278 | 42.793 | 1.00 | 0.00 | 0.440 | 0.00 | 0.00 |
| ATOM | 4145 | CA | ASP | 359P | −3.793 | 2.572 | 43.353 | 1.00 | 0.00 | 0.158 | 9.40 | 4.00 |
| ATOM | 4146 | HA | ASP | 359P | −3.585 | 2.179 | 44.348 | 1.00 | 0.00 | 0.053 | 0.00 | 0.00 |
| ATOM | 4147 | CB | ASP | 359P | −4.130 | 1.367 | 42.478 | 1.00 | 0.00 | −0.336 | 12.77 | 4.00 |
| ATOM | 4148 | HB1 | ASP | 359P | −3.271 | 0.713 | 42.323 | 1.00 | 0.00 | 0.053 | 0.00 | 0.00 |
| ATOM | 4149 | HB2 | ASP | 359P | −4.478 | 1.658 | 41.487 | 1.00 | 0.00 | 0.053 | 0.00 | 0.00 |
| ATOM | 4150 | CG | ASP | 359P | −5.225 | 0.502 | 43.081 | 1.00 | 0.00 | 0.297 | 9.82 | 4.00 |
| ATOM | 4151 | OD1 | ASP | 359P | −5.704 | 0.839 | 44.189 | 1.00 | 0.00 | −0.534 | 8.17 | −18.95 |
| ATOM | 4152 | OD2 | ASP | 359P | −5.603 | −0.517 | 42.454 | 1.00 | 0.00 | −0.534 | 8.17 | −18.95 |
| ATOM | 4153 | C | ASP | 359P | −2.505 | 3.239 | 42.856 | 1.00 | 0.00 | 0.396 | 9.82 | 4.00 |
| ATOM | 4154 | O | ASP | 359P | −1.500 | 3.234 | 43.563 | 1.00 | 0.00 | −0.396 | 8.17 | −17.40 |
| ATOM | 4155 | N | PHE | 360 | −2.528 | 3.823 | 41.660 | 1.00 | 0.00 | −0.650 | 9.00 | −17.40 |
| ATOM | 4156 | HN | PHE | 360 | −3.398 | 3.838 | 41.110 | 1.00 | 0.00 | 0.440 | 0.00 | 0.00 |
| ATOM | 4157 | CA | PHE | 360 | −1.318 | 4.441 | 41.130 | 1.00 | 0.00 | 0.158 | 9.40 | 4.00 |
| ATOM | 4158 | HA | PHE | 360 | −0.497 | 3.735 | 40.998 | 1.00 | 0.00 | 0.053 | 0.00 | 0.00 |
| ATOM | 4159 | CB | PHE | 360 | −1.537 | 5.003 | 39.726 | 1.00 | 0.00 | −0.106 | 12.77 | 4.00 |
| ATOM | 4160 | HB1 | PHE | 360 | −2.168 | 5.891 | 39.717 | 1.00 | 0.00 | 0.053 | 0.00 | 0.00 |
| ATOM | 4161 | HB2 | PHE | 360 | −2.016 | 4.292 | 39.053 | 1.00 | 0.00 | 0.053 | 0.00 | 0.00 |
| ATOM | 4162 | CG | PHE | 360 | −0.253 | 5.407 | 39.047 | 1.00 | 0.00 | 0.000 | 7.26 | 0.60 |
| ATOM | 4163 | CD1 | PHE | 360 | 0.640 | 4.437 | 38.596 | 1.00 | 0.00 | −0.127 | 10.80 | 0.60 |
| ATOM | 4164 | HD1 | PHE | 360 | 0.376 | 3.382 | 38.680 | 1.00 | 0.00 | 0.127 | 0.00 | 0.00 |
| ATOM | 4165 | CD2 | PHE | 360 | 0.093 | 6.748 | 38.928 | 1.00 | 0.00 | −0.127 | 10.80 | 0.60 |
| ATOM | 4166 | HD2 | PHE | 360 | −0.601 | 7.512 | 39.275 | 1.00 | 0.00 | 0.127 | 0.00 | 0.00 |
| ATOM | 4167 | CE1 | PHE | 360 | 1.870 | 4.797 | 38.037 | 1.00 | 0.00 | −0.127 | 10.80 | 0.60 |
| ATOM | 4168 | HE1 | PHE | 360 | 2.561 | 4.029 | 37.690 | 1.00 | 0.00 | 0.127 | 0.00 | 0.00 |
| ATOM | 4169 | CE2 | PHE | 360 | 1.318 | 7.134 | 38.370 | 1.00 | 0.00 | −0.127 | 10.80 | 0.60 |
| ATOM | 4170 | HE2 | PHE | 360 | 1.570 | 8.190 | 38.285 | 1.00 | 0.00 | 0.127 | 0.00 | 0.00 |
| ATOM | 4171 | CZ | PHE | 360 | 2.211 | 6.167 | 37.925 | 1.00 | 0.00 | −0.127 | 10.80 | 0.60 |
| ATOM | 4172 | HZ | PHE | 360 | 3.167 | 6.460 | 37.492 | 1.00 | 0.00 | 0.127 | 0.00 | 0.00 |
| ATOM | 4173 | C | PHE | 360 | −0.766 | 5.546 | 42.026 | 1.00 | 0.00 | 0.396 | 9.82 | 4.00 |
| ATOM | 4174 | O | PHE | 360 | 0.426 | 5.587 | 42.289 | 1.00 | 0.00 | −0.396 | 8.17 | −17.40 |
| ATOM | 4175 | N | LEU | 361 | −1.630 | 6.444 | 42.484 | 1.00 | 0.00 | −0.650 | 9.00 | −17.40 |
| ATOM | 4176 | HN | LEU | 361 | −2.619 | 6.369 | 42.207 | 1.00 | 0.00 | 0.440 | 0.00 | 0.00 |
| ATOM | 4177 | CA | LEU | 361 | −1.221 | 7.542 | 43.373 | 1.00 | 0.00 | 0.158 | 9.40 | 4.00 |
| ATOM | 4178 | HA | LEU | 361 | −0.443 | 8.156 | 42.920 | 1.00 | 0.00 | 0.053 | 0.00 | 0.00 |
| ATOM | 4179 | CB | LEU | 361 | −2.413 | 8.473 | 43.653 | 1.00 | 0.00 | −0.106 | 12.77 | 4.00 |
| ATOM | 4180 | HB1 | LEU | 361 | −2.186 | 9.032 | 44.560 | 1.00 | 0.00 | 0.053 | 0.00 | 0.00 |
| ATOM | 4181 | HB2 | LEU | 361 | −3.295 | 7.846 | 43.783 | 1.00 | 0.00 | 0.053 | 0.00 | 0.00 |
| ATOM | 4182 | CG | LEU | 361 | −2.768 | 9.507 | 42.582 | 1.00 | 0.00 | −0.053 | 9.40 | 4.00 |
| ATOM | 4183 | HG | LEU | 361 | −3.651 | 10.052 | 42.915 | 1.00 | 0.00 | 0.053 | 0.00 | 0.00 |
| ATOM | 4184 | CD1 | LEU | 361 | −1.579 | 10.451 | 42.419 | 1.00 | 0.00 | −0.159 | 16.15 | 4.00 |
| ATOM | 4185 | HD1 | LEU | 361 | −1.809 | 11.198 | 41.659 | 1.00 | 0.00 | 0.053 | 0.00 | 0.00 |
| ATOM | 4186 | HD1 | LEU | 361 | −1.376 | 10.948 | 43.367 | 1.00 | 0.00 | 0.053 | 0.00 | 0.00 |
| ATOM | 4187 | HD1 | LEU | 361 | −0.701 | 9.881 | 42.113 | 1.00 | 0.00 | 0.053 | 0.00 | 0.00 |
| ATOM | 4188 | CD2 | LEU | 361 | −3.087 | 8.829 | 41.257 | 1.00 | 0.00 | −0.159 | 16.15 | 4.00 |
| ATOM | 4189 | HD2 | LEU | 361 | −3.336 | 9.584 | 40.512 | 1.00 | 0.00 | 0.053 | 0.00 | 0.00 |
| ATOM | 4190 | HD2 | LEU | 361 | −2.219 | 8.260 | 40.921 | 1.00 | 0.00 | 0.053 | 0.00 | 0.00 |
| ATOM | 4191 | HD2 | LEU | 361 | −3.933 | 8.155 | 41.387 | 1.00 | 0.00 | 0.053 | 0.00 | 0.00 |
| ATOM | 4192 | C | LEU | 361 | −0.670 | 7.018 | 44.698 | 1.00 | 0.00 | 0.396 | 9.82 | 4.00 |
| ATOM | 4193 | O | LEU | 361 | 0.283 | 7.569 | 45.256 | 1.00 | 0.00 | −0.396 | 8.17 | −17.40 |
| ATOM | 4194 | N | ALA | 362 | −1.268 | 5.952 | 45.209 | 1.00 | 0.00 | −0.650 | 9.00 | −17.40 |
| ATOM | 4195 | HN | ALA | 362 | −2.073 | 5.532 | 44.722 | 1.00 | 0.00 | 0.440 | 0.00 | 0.00 |
| ATOM | 4196 | CA | ALA | 362 | −0.790 | 5.368 | 46.461 | 1.00 | 0.00 | 0.158 | 9.40 | 4.00 |
| ATOM | 4197 | HA | ALA | 362 | −0.770 | 6.170 | 47.198 | 1.00 | 0.00 | 0.053 | 0.00 | 0.00 |
| ATOM | 4198 | CB | ALA | 362 | −1.741 | 4.251 | 46.938 | 1.00 | 0.00 | −0.159 | 16.15 | 4.00 |
| ATOM | 4199 | HB1 | ALA | 362 | −1.367 | 3.829 | 47.871 | 1.00 | 0.00 | 0.053 | 0.00 | 0.00 |
| ATOM | 4200 | HB2 | ALA | 362 | −2.736 | 4.664 | 47.099 | 1.00 | 0.00 | 0.053 | 0.00 | 0.00 |
| ATOM | 4201 | HB3 | ALA | 362 | −1.791 | 3.468 | 46.181 | 1.00 | 0.00 | 0.053 | 0.00 | 0.00 |
| ATOM | 4202 | C | ALA | 362 | 0.607 | 4.811 | 46.188 | 1.00 | 0.00 | 0.396 | 9.82 | 4.00 |
| ATOM | 4203 | O | ALA | 362 | 1.522 | 4.960 | 46.991 | 1.00 | 0.00 | −0.396 | 8.17 | −17.40 |
| ATOM | 4204 | N | ARG | 363G | 0.765 | 4.201 | 45.020 | 1.00 | 0.00 | −0.650 | 9.00 | −17.40 |
| ATOM | 4205 | HN | ARG | 363G | −0.043 | 4.123 | 44.386 | 1.00 | 0.00 | 0.440 | 0.00 | 0.00 |
| ATOM | 4206 | CA | ARG | 363G | 2.042 | 3.637 | 44.603 | 1.00 | 0.00 | 0.158 | 9.40 | 4.00 |
| ATOM | 4207 | HA | ARG | 363G | 2.344 | 2.847 | 45.291 | 1.00 | 0.00 | 0.053 | 0.00 | 0.00 |
| ATOM | 4208 | CB | ARG | 363G | 1.894 | 3.044 | 43.195 | 1.00 | 0.00 | −0.106 | 12.77 | 4.00 |
| ATOM | 4209 | HB1 | ARG | 363G | 1.839 | 3.804 | 42.416 | 1.00 | 0.00 | 0.053 | 0.00 | 0.00 |
| ATOM | 4210 | HB2 | ARG | 363G | 0.994 | 2.438 | 43.080 | 1.00 | 0.00 | 0.053 | 0.00 | 0.00 |
| ATOM | 4211 | CG | ARG | 363G | 3.037 | 2.151 | 42.812 | 1.00 | 0.00 | −0.106 | 12.77 | 4.00 |
| ATOM | 4212 | HG1 | ARG | 363G | 2.901 | 1.126 | 43.157 | 1.00 | 0.00 | 0.053 | 0.00 | 0.00 |
| ATOM | 4213 | HG2 | ARG | 363G | 3.987 | 2.488 | 43.225 | 1.00 | 0.00 | 0.053 | 0.00 | 0.00 |
| ATOM | 4214 | CD | ARG | 363G | 3.235 | 2.068 | 41.298 | 1.00 | 0.00 | 0.374 | 12.77 | 4.00 |
| ATOM | 4215 | HD1 | ARG | 363G | 3.480 | 3.058 | 40.915 | 1.00 | 0.00 | 0.053 | 0.00 | 0.00 |
| ATOM | 4216 | HD2 | ARG | 363G | 2.314 | 1.711 | 40.836 | 1.00 | 0.00 | 0.053 | 0.00 | 0.00 |
| ATOM | 4217 | NE | ARG | 363G | 4.324 | 1.142 | 41.009 | 1.00 | 0.00 | −0.819 | 9.00 | −24.67 |
| ATOM | 4218 | HE | ARG | 363G | 5.238 | 1.543 | 40.759 | 1.00 | 0.00 | 0.407 | 0.00 | 0.00 |
| ATOM | 4219 | CZ | ARG | 363G | 4.221 | −0.187 | 41.045 | 1.00 | 0.00 | 0.796 | 6.95 | 4.00 |
| ATOM | 4220 | NH1 | ARG | 363G | 3.066 | −0.764 | 41.347 | 1.00 | 0.00 | −0.746 | 9.00 | −24.67 |
| ATOM | 4221 | HH1 | ARG | 363G | 2.994 | −1.791 | 41.372 | 1.00 | 0.00 | 0.407 | 0.00 | 0.00 |
| ATOM | 4222 | HH1 | ARG | 363G | 2.240 | −0.185 | 41.556 | 1.00 | 0.00 | 0.407 | 0.00 | 0.00 |
| ATOM | 4223 | NH2 | ARG | 363G | 5.287 | −0.939 | 40.800 | 1.00 | 0.00 | −0.746 | 9.00 | −24.67 |
| ATOM | 4224 | HH2 | ARG | 363G | 5.210 | −1.965 | 40.827 | 1.00 | 0.00 | 0.407 | 0.00 | 0.00 |
| ATOM | 4225 | HH2 | ARG | 363G | 6.191 | −0.495 | 40.582 | 1.00 | 0.00 | 0.407 | 0.00 | 0.00 |
| ATOM | 4226 | C | ARG | 363G | 3.126 | 4.733 | 44.594 | 1.00 | 0.00 | 0.396 | 9.82 | 4.00 |
| ATOM | 4227 | O | ARG | 363G | 4.204 | 4.574 | 45.169 | 1.00 | 0.00 | −0.396 | 8.17 | −17.40 |
| ATOM | 4228 | N | VAL | 364 | 2.801 | 5.853 | 43.956 | 1.00 | 0.00 | −0.650 | 9.00 | −17.40 |
| ATOM | 4229 | HN | VAL | 364 | 1.864 | 5.915 | 43.531 | 1.00 | 0.00 | 0.440 | 0.00 | 0.00 |
| ATOM | 4230 | CA | VAL | 364 | 3.698 | 7.004 | 43.822 | 1.00 | 0.00 | 0.158 | 9.40 | 4.00 |
| ATOM | 4231 | HA | VAL | 364 | 4.650 | 6.756 | 43.352 | 1.00 | 0.00 | 0.053 | 0.00 | 0.00 |
| ATOM | 4232 | CB | VAL | 364 | 3.061 | 8.072 | 42.898 | 1.00 | 0.00 | −0.053 | 9.40 | 4.00 |
| ATOM | 4233 | HB | VAL | 364 | 2.041 | 8.301 | 43.206 | 1.00 | 0.00 | 0.053 | 0.00 | 0.00 |
| ATOM | 4234 | CG1 | VAL | 364 | 3.873 | 9.396 | 42.928 | 1.00 | 0.00 | −0.159 | 16.15 | 4.00 |
| ATOM | 4235 | HG1 | VAL | 364 | 3.402 | 10.126 | 42.270 | 1.00 | 0.00 | 0.053 | 0.00 | 0.00 |
| ATOM | 4236 | HG1 | VAL | 364 | 3.895 | 9.785 | 43.945 | 1.00 | 0.00 | 0.053 | 0.00 | 0.00 |
| ATOM | 4237 | HG1 | VAL | 364 | 4.891 | 9.206 | 42.589 | 1.00 | 0.00 | 0.053 | 0.00 | 0.00 |
| ATOM | 4238 | CG2 | VAL | 364 | 2.974 | 7.533 | 41.510 | 1.00 | 0.00 | −0.159 | 16.15 | 4.00 |
| ATOM | 4239 | HG2 | VAL | 364 | 2.526 | 8.281 | 40.856 | 1.00 | 0.00 | 0.053 | 0.00 | 0.00 |
| ATOM | 4240 | HG2 | VAL | 364 | 3.973 | 7.289 | 41.151 | 1.00 | 0.00 | 0.053 | 0.00 | 0.00 |
| ATOM | 4241 | HG2 | VAL | 364 | 2.358 | 6.633 | 41.507 | 1.00 | 0.00 | 0.053 | 0.00 | 0.00 |
| ATOM | 4242 | C | VAL | 364 | 4.061 | 7.646 | 45.159 | 1.00 | 0.00 | 0.396 | 9.82 | 4.00 |
| ATOM | 4243 | O | VAL | 364 | 5.244 | 7.876 | 45.446 | 1.00 | 0.00 | −0.396 | 8.17 | −17.40 |
| ATOM | 4244 | N | TYR | 365 | 3.056 | 7.951 | 45.974 | 1.00 | 0.00 | −0.650 | 9.00 | −17.40 |
| ATOM | 4245 | HN | TYR | 365 | 2.083 | 7.746 | 45.702 | 1.00 | 0.00 | 0.440 | 0.00 | 0.00 |
| ATOM | 4246 | CA | TYR | 365 | 3.346 | 8.575 | 47.250 | 1.00 | 0.00 | 0.158 | 9.40 | 4.00 |
| ATOM | 4247 | HA | TYR | 365 | 4.178 | 9.265 | 47.112 | 1.00 | 0.00 | 0.053 | 0.00 | 0.00 |
| ATOM | 4248 | CB | TYR | 365 | 2.184 | 9.481 | 47.669 | 1.00 | 0.00 | −0.106 | 12.77 | 4.00 |
| ATOM | 4249 | HB1 | TYR | 365 | 2.301 | 9.814 | 48.700 | 1.00 | 0.00 | 0.053 | 0.00 | 0.00 |
| ATOM | 4250 | HB2 | TYR | 365 | 1.232 | 8.955 | 47.592 | 1.00 | 0.00 | 0.053 | 0.00 | 0.00 |
| ATOM | 4251 | CG | TYR | 365 | 2.110 | 10.710 | 46.787 | 1.00 | 0.00 | 0.000 | 7.26 | 0.60 |
| ATOM | 4252 | CD1 | TYR | 365 | 1.295 | 10.743 | 45.653 | 1.00 | 0.00 | −0.127 | 10.80 | 0.60 |
| ATOM | 4253 | HD1 | TYR | 365 | 0.638 | 9.899 | 45.440 | 1.00 | 0.00 | 0.127 | 0.00 | 0.00 |
| ATOM | 4254 | CE1 | TYR | 365 | 1.308 | 11.841 | 44.785 | 1.00 | 0.00 | −0.127 | 10.80 | 0.60 |
| ATOM | 4255 | HE1 | TYR | 365 | 0.665 | 11.855 | 43.904 | 1.00 | 0.00 | 0.127 | 0.00 | 0.00 |
| ATOM | 4256 | CD2 | TYR | 365 | 2.933 | 11.802 | 47.035 | 1.00 | 0.00 | −0.127 | 10.80 | 0.60 |
| ATOM | 4257 | HD2 | TYR | 365 | 3.571 | 11.799 | 47.918 | 1.00 | 0.00 | 0.127 | 0.00 | 0.00 |
| ATOM | 4258 | CE2 | TYR | 365 | 2.960 | 12.902 | 46.177 | 1.00 | 0.00 | −0.127 | 10.80 | 0.60 |
| ATOM | 4259 | HE2 | TYR | 365 | 3.616 | 13.745 | 46.389 | 1.00 | 0.00 | 0.127 | 0.00 | 0.00 |
| ATOM | 4260 | CZ | TYR | 365 | 2.153 | 12.917 | 45.060 | 1.00 | 0.00 | 0.026 | 7.26 | 0.60 |
| ATOM | 4261 | OH | TYR | 365 | 2.218 | 14.004 | 44.214 | 1.00 | 0.00 | −0.451 | 10.94 | −17.40 |
| ATOM | 4262 | HH | TYR | 365 | 2.999 | 14.612 | 44.496 | 1.00 | 0.00 | 0.424 | 0.00 | 0.00 |
| ATOM | 4263 | C | TYR | 365 | 3.733 | 7.599 | 48.349 | 1.00 | 0.00 | 0.396 | 9.82 | 4.00 |
| ATOM | 4264 | O | TYR | 365 | 4.034 | 8.009 | 49.478 | 1.00 | 0.00 | −0.396 | 8.17 | −17.40 |
| ATOM | 4265 | N | GLY | 366 | 3.737 | 6.316 | 48.010 | 1.00 | 0.00 | −0.650 | 9.00 | −17.40 |
| ATOM | 4266 | HN | GLY | 366 | 3.439 | 6.044 | 47.062 | 1.00 | 0.00 | 0.440 | 0.00 | 0.00 |
| ATOM | 4267 | CA | GLY | 366 | 4.152 | 5.286 | 48.947 | 1.00 | 0.00 | 0.105 | 9.40 | 4.00 |
| ATOM | 4268 | HA1 | GLY | 366 | 3.534 | 4.405 | 48.772 | 1.00 | 0.00 | 0.053 | 0.00 | 0.00 |
| ATOM | 4269 | HA2 | GLY | 366 | 4.004 | 5.671 | 49.955 | 1.00 | 0.00 | 0.053 | 0.00 | 0.00 |
| ATOM | 4270 | C | GLY | 366 | 5.627 | 4.981 | 48.687 | 1.00 | 0.00 | 0.396 | 9.82 | 4.00 |
| ATOM | 4271 | O | GLY | 366 | 6.329 | 4.425 | 49.532 | 1.00 | 0.00 | −0.396 | 8.17 | −17.40 |
| ATOM | 4272 | N | ALA | 367 | 6.102 | 5.364 | 47.504 | 1.00 | 0.00 | −0.650 | 9.00 | −17.40 |
| ATOM | 4273 | HN | ALA | 367 | 5.471 | 5.831 | 46.837 | 1.00 | 0.00 | 0.440 | 0.00 | 0.00 |
| ATOM | 4274 | CA | ALA | 367 | 7.491 | 5.139 | 47.129 | 1.00 | 0.00 | 0.158 | 9.40 | 4.00 |
| ATOM | 4275 | HA | ALA | 367 | 7.787 | 4.111 | 47.340 | 1.00 | 0.00 | 0.053 | 0.00 | 0.00 |
| ATOM | 4276 | CB | ALA | 367 | 7.671 | 5.366 | 45.622 | 1.00 | 0.00 | −0.159 | 16.15 | 4.00 |
| ATOM | 4277 | HB1 | ALA | 367 | 8.712 | 5.195 | 45.350 | 1.00 | 0.00 | 0.053 | 0.00 | 0.00 |
| ATOM | 4278 | HB2 | ALA | 367 | 7.034 | 4.673 | 45.071 | 1.00 | 0.00 | 0.053 | 0.00 | 0.00 |
| ATOM | 4279 | HB3 | ALA | 367 | 7.393 | 6.390 | 45.372 | 1.00 | 0.00 | 0.053 | 0.00 | 0.00 |
| ATOM | 4280 | C | ALA | 367 | 8.417 | 6.067 | 47.903 | 1.00 | 0.00 | 0.396 | 9.82 | 4.00 |
| ATOM | 4281 | O | ALA | 367 | 8.072 | 7.219 | 48.187 | 1.00 | 0.00 | −0.396 | 8.17 | −17.40 |
| ATOM | 4282 | N | PRO | 368 | 9.620 | 5.586 | 48.239 | 1.00 | 0.00 | −0.422 | 9.00 | −17.40 |
| ATOM | 4283 | CD | PRO | 368 | 10.241 | 4.281 | 47.948 | 1.00 | 0.00 | 0.105 | 12.77 | 4.00 |
| ATOM | 4284 | HD1 | PRO | 368 | 10.031 | 3.959 | 46.927 | 1.00 | 0.00 | 0.053 | 0.00 | 0.00 |
| ATOM | 4285 | HD2 | PRO | 368 | 9.869 | 3.505 | 48.618 | 1.00 | 0.00 | 0.053 | 0.00 | 0.00 |
| ATOM | 4286 | CA | PRO | 368 | 10.541 | 6.441 | 48.976 | 1.00 | 0.00 | 0.158 | 9.40 | 4.00 |
| ATOM | 4287 | HA | PRO | 368 | 10.086 | 6.838 | 49.883 | 1.00 | 0.00 | 0.053 | 0.00 | 0.00 |
| ATOM | 4288 | CB | PRO | 368 | 11.682 | 5.488 | 49.320 | 1.00 | 0.00 | −0.106 | 12.77 | 4.00 |
| ATOM | 4289 | HB1 | PRO | 368 | 11.484 | 4.959 | 50.252 | 1.00 | 0.00 | 0.053 | 0.00 | 0.00 |
| ATOM | 4290 | HB2 | PRO | 368 | 12.622 | 6.026 | 49.436 | 1.00 | 0.00 | 0.053 | 0.00 | 0.00 |
| ATOM | 4291 | CG | PRO | 368 | 11.710 | 4.578 | 48.169 | 1.00 | 0.00 | −0.106 | 12.77 | 4.00 |
| ATOM | 4292 | HG1 | PRO | 368 | 12.281 | 3.677 | 48.392 | 1.00 | 0.00 | 0.053 | 0.00 | 0.00 |
| ATOM | 4293 | HG2 | PRO | 368 | 12.167 | 5.053 | 47.301 | 1.00 | 0.00 | 0.053 | 0.00 | 0.00 |
| ATOM | 4294 | C | PRO | 368 | 10.994 | 7.635 | 48.144 | 1.00 | 0.00 | 0.396 | 9.82 | 4.00 |
| ATOM | 4295 | O | PRO | 368 | 11.062 | 7.574 | 46.917 | 1.00 | 0.00 | −0.396 | 8.17 | −17.40 |
| ATOM | 4296 | N | GLN | 369 | 11.305 | 8.724 | 48.832 | 1.00 | 0.00 | −0.650 | 9.00 | −17.40 |
| ATOM | 4297 | HN | GLN | 369 | 11.218 | 8.703 | 49.858 | 1.00 | 0.00 | 0.440 | 0.00 | 0.00 |
| ATOM | 4298 | CA | GLN | 369 | 11.764 | 9.949 | 48.193 | 1.00 | 0.00 | 0.158 | 9.40 | 4.00 |
| ATOM | 4299 | HA | GLN | 369 | 11.325 | 9.998 | 47.196 | 1.00 | 0.00 | 0.053 | 0.00 | 0.00 |
| ATOM | 4300 | CB | GLN | 369 | 11.330 | 11.140 | 49.035 | 1.00 | 0.00 | −0.106 | 12.77 | 4.00 |
| ATOM | 4301 | HB1 | GLN | 369 | 12.043 | 11.259 | 49.850 | 1.00 | 0.00 | 0.053 | 0.00 | 0.00 |
| ATOM | 4302 | HB2 | GLN | 369 | 10.332 | 10.937 | 49.424 | 1.00 | 0.00 | 0.053 | 0.00 | 0.00 |
| ATOM | 4303 | CG | GLN | 369 | 11.271 | 12.448 | 48.284 | 1.00 | 0.00 | −0.106 | 12.77 | 4.00 |
| ATOM | 4304 | HG1 | GLN | 369 | 10.707 | 12.363 | 47.355 | 1.00 | 0.00 | 0.053 | 0.00 | 0.00 |
| ATOM | 4305 | HG2 | GLN | 369 | 12.262 | 12.812 | 48.016 | 1.00 | 0.00 | 0.053 | 0.00 | 0.00 |
| ATOM | 4306 | CD | GLN | 369 | 10.607 | 13.547 | 49.099 | 1.00 | 0.00 | 0.396 | 9.82 | 4.00 |
| ATOM | 4307 | OE1 | GLN | 369 | 9.521 | 13.351 | 49.669 | 1.00 | 0.00 | −0.396 | 8.17 | −17.40 |
| ATOM | 4308 | NE2 | GLN | 369 | 11.247 | 14.712 | 49.153 | 1.00 | 0.00 | −0.879 | 13.25 | −17.40 |
| ATOM | 4309 | HE2 | GLN | 369 | 12.146 | 14.828 | 48.664 | 1.00 | 0.00 | 0.440 | 0.00 | 0.00 |
| ATOM | 4310 | HE2 | GLN | 369 | 10.843 | 15.497 | 49.683 | 1.00 | 0.00 | 0.440 | 0.00 | 0.00 |
| ATOM | 4311 | C | GLN | 369 | 13.289 | 9.903 | 48.110 | 1.00 | 0.00 | 0.396 | 9.82 | 4.00 |
| ATOM | 4312 | O | GLN | 369 | 13.945 | 9.482 | 49.057 | 1.00 | 0.00 | −0.396 | 8.17 | −17.40 |
| ATOM | 4313 | N | LEU | 370 | 13.854 | 10.314 | 46.980 | 1.00 | 0.00 | −0.650 | 9.00 | −17.40 |
| ATOM | 4314 | HN | LEU | 370 | 13.261 | 10.636 | 46.202 | 1.00 | 0.00 | 0.440 | 0.00 | 0.00 |
| ATOM | 4315 | CA | LEU | 370 | 15.305 | 10.313 | 46.829 | 1.00 | 0.00 | 0.158 | 9.40 | 4.00 |
| ATOM | 4316 | HA | LEU | 370 | 15.775 | 10.181 | 47.803 | 1.00 | 0.00 | 0.053 | 0.00 | 0.00 |
| ATOM | 4317 | CB | LEU | 370 | 15.756 | 9.136 | 45.936 | 1.00 | 0.00 | −0.106 | 12.77 | 4.00 |
| ATOM | 4318 | HB1 | LEU | 370 | 15.453 | 9.366 | 44.914 | 1.00 | 0.00 | 0.053 | 0.00 | 0.00 |
| ATOM | 4319 | HB2 | LEU | 370 | 15.261 | 8.237 | 46.305 | 1.00 | 0.00 | 0.053 | 0.00 | 0.00 |
| ATOM | 4320 | CG | LEU | 370 | 17.256 | 8.814 | 45.882 | 1.00 | 0.00 | −0.053 | 9.40 | 4.00 |
| ATOM | 4321 | HG | LEU | 370 | 17.788 | 9.691 | 45.515 | 1.00 | 0.00 | 0.053 | 0.00 | 0.00 |
| ATOM | 4322 | CD1 | LEU | 370 | 17.733 | 8.456 | 47.296 | 1.00 | 0.00 | −0.159 | 16.15 | 4.00 |
| ATOM | 4323 | HD1 | LEU | 370 | 18.797 | 8.224 | 47.273 | 1.00 | 0.00 | 0.053 | 0.00 | 0.00 |
| ATOM | 4324 | HD1 | LEU | 370 | 17.559 | 9.300 | 47.963 | 1.00 | 0.00 | 0.053 | 0.00 | 0.00 |
| ATOM | 4325 | HD1 | LEU | 370 | 17.180 | 7.588 | 47.657 | 1.00 | 0.00 | 0.053 | 0.00 | 0.00 |
| ATOM | 4326 | CD2 | LEU | 370 | 17.544 | 7.661 | 44.918 | 1.00 | 0.00 | −0.159 | 16.15 | 4.00 |
| ATOM | 4327 | HD2 | LEU | 370 | 18.614 | 7.458 | 44.903 | 1.00 | 0.00 | 0.053 | 0.00 | 0.00 |
| ATOM | 4328 | HD2 | LEU | 370 | 17.010 | 6.769 | 45.247 | 1.00 | 0.00 | 0.053 | 0.00 | 0.00 |
| ATOM | 4329 | HD2 | LEU | 370 | 17.212 | 7.933 | 43.916 | 1.00 | 0.00 | 0.053 | 0.00 | 0.00 |
| ATOM | 4330 | C | LEU | 370 | 15.761 | 11.643 | 46.223 | 1.00 | 0.00 | 0.396 | 9.82 | 4.00 |
| ATOM | 4331 | O | LEU | 370 | 15.113 | 12.183 | 45.317 | 1.00 | 0.00 | −0.396 | 8.17 | −17.40 |
| ATOM | 4332 | N | GLN | 371 | 16.867 | 12.177 | 46.739 | 1.00 | 0.00 | −0.650 | 9.00 | −17.40 |
| ATOM | 4333 | HN | GLN | 371 | 17.348 | 11.688 | 47.507 | 1.00 | 0.00 | 0.440 | 0.00 | 0.00 |
| ATOM | 4334 | CA | GLN | 371 | 17.415 | 13.437 | 46.243 | 1.00 | 0.00 | 0.158 | 9.40 | 4.00 |
| ATOM | 4335 | HA | GLN | 371 | 16.683 | 14.227 | 46.410 | 1.00 | 0.00 | 0.053 | 0.00 | 0.00 |
| ATOM | 4336 | CB | GLN | 371 | 18.704 | 13.769 | 46.990 | 1.00 | 0.00 | −0.106 | 12.77 | 4.00 |
| ATOM | 4337 | HB1 | GLN | 371 | 19.321 | 14.515 | 46.490 | 1.00 | 0.00 | 0.053 | 0.00 | 0.00 |
| ATOM | 4338 | HB2 | GLN | 371 | 19.362 | 12.911 | 47.133 | 1.00 | 0.00 | 0.053 | 0.00 | 0.00 |
| ATOM | 4339 | CG | GLN | 371 | 18.479 | 14.319 | 48.375 | 1.00 | 0.00 | −0.106 | 12.77 | 4.00 |
| ATOM | 4340 | HG1 | GLN | 371 | 17.507 | 14.038 | 48.782 | 1.00 | 0.00 | 0.053 | 0.00 | 0.00 |
| ATOM | 4341 | HG2 | GLN | 371 | 18.519 | 15.407 | 48.402 | 1.00 | 0.00 | 0.053 | 0.00 | 0.00 |
| ATOM | 4342 | CD | GLN | 371 | 19.514 | 13.830 | 49.368 | 1.00 | 0.00 | 0.396 | 9.82 | 4.00 |
| ATOM | 4343 | OE1 | GLN | 371 | 20.712 | 13.784 | 49.065 | 1.00 | 0.00 | −0.396 | 8.17 | −17.40 |
| ATOM | 4344 | NE2 | GLN | 371 | 19.059 | 13.468 | 50.569 | 1.00 | 0.00 | −0.879 | 13.25 | −17.40 |
| ATOM | 4345 | HE2 | GLN | 371 | 18.051 | 13.523 | 50.776 | 1.00 | 0.00 | 0.440 | 0.00 | 0.00 |
| ATOM | 4346 | HE2 | GLN | 371 | 19.715 | 13.133 | 51.289 | 1.00 | 0.00 | 0.440 | 0.00 | 0.00 |
| ATOM | 4347 | C | GLN | 371 | 17.702 | 13.294 | 44.759 | 1.00 | 0.00 | 0.396 | 9.82 | 4.00 |
| ATOM | 4348 | O | GLN | 371 | 18.278 | 12.299 | 44.337 | 1.00 | 0.00 | −0.396 | 8.17 | −17.40 |
| ATOM | 4349 | N | VAL | 372 | 17.313 | 14.285 | 43.964 | 1.00 | 0.00 | −0.650 | 9.00 | −17.40 |
| ATOM | 4350 | HN | VAL | 372 | 16.847 | 15.112 | 44.363 | 1.00 | 0.00 | 0.440 | 0.00 | 0.00 |
| ATOM | 4351 | CA | VAL | 372 | 17.544 | 14.203 | 42.524 | 1.00 | 0.00 | 0.158 | 9.40 | 4.00 |
| ATOM | 4352 | HA | VAL | 372 | 16.905 | 13.471 | 42.028 | 1.00 | 0.00 | 0.053 | 0.00 | 0.00 |
| ATOM | 4353 | CB | VAL | 372 | 17.170 | 15.545 | 41.825 | 1.00 | 0.00 | −0.053 | 9.40 | 4.00 |
| ATOM | 4354 | HB | VAL | 372 | 16.146 | 15.831 | 42.065 | 1.00 | 0.00 | 0.053 | 0.00 | 0.00 |
| ATOM | 4355 | CG1 | VAL | 372 | 18.098 | 16.665 | 42.280 | 1.00 | 0.00 | −0.159 | 16.15 | 4.00 |
| ATOM | 4356 | HG1 | VAL | 372 | 17.820 | 17.593 | 41.780 | 1.00 | 0.00 | 0.053 | 0.00 | 0.00 |
| ATOM | 4357 | HG1 | VAL | 372 | 18.011 | 16.793 | 43.359 | 1.00 | 0.00 | 0.053 | 0.00 | 0.00 |
| ATOM | 4358 | HG1 | VAL | 372 | 19.127 | 16.410 | 42.026 | 1.00 | 0.00 | 0.053 | 0.00 | 0.00 |
| ATOM | 4359 | CG2 | VAL | 372 | 17.207 | 15.373 | 40.308 | 1.00 | 0.00 | −0.159 | 16.15 | 4.00 |
| ATOM | 4360 | HG2 | VAL | 372 | 16.944 | 16.315 | 39.828 | 1.00 | 0.00 | 0.053 | 0.00 | 0.00 |
| ATOM | 4361 | HG2 | VAL | 372 | 18.209 | 15.076 | 40.000 | 1.00 | 0.00 | 0.053 | 0.00 | 0.00 |
| ATOM | 4362 | HG2 | VAL | 372 | 16.493 | 14.604 | 40.011 | 1.00 | 0.00 | 0.053 | 0.00 | 0.00 |
| ATOM | 4363 | C | VAL | 372 | 18.987 | 13.803 | 42.164 | 1.00 | 0.00 | 0.396 | 9.82 | 4.00 |
| ATOM | 4364 | O | VAL | 372 | 19.217 | 12.958 | 41.291 | 1.00 | 0.00 | −0.396 | 8.17 | −17.40 |
| ATOM | 4365 | N | GLU | 373 | 19.954 | 14.385 | 42.862 | 1.00 | 0.00 | −0.650 | 9.00 | −17.40 |
| ATOM | 4366 | HN | GLU | 373 | 19.702 | 15.055 | 43.602 | 1.00 | 0.00 | 0.440 | 0.00 | 0.00 |
| ATOM | 4367 | CA | GLU | 373 | 21.363 | 14.103 | 42.612 | 1.00 | 0.00 | 0.158 | 9.40 | 4.00 |
| ATOM | 4368 | HA | GLU | 373 | 21.651 | 14.351 | 41.590 | 1.00 | 0.00 | 0.053 | 0.00 | 0.00 |
| ATOM | 4369 | CB | GLU | 373 | 22.221 | 14.977 | 43.536 | 1.00 | 0.00 | −0.106 | 12.77 | 4.00 |
| ATOM | 4370 | HB1 | GLU | 373 | 22.078 | 14.624 | 44.557 | 1.00 | 0.00 | 0.053 | 0.00 | 0.00 |
| ATOM | 4371 | HB2 | GLU | 373 | 21.885 | 16.008 | 43.428 | 1.00 | 0.00 | 0.053 | 0.00 | 0.00 |
| ATOM | 4372 | CG | GLU | 373 | 23.717 | 14.965 | 43.265 | 1.00 | 0.00 | −0.106 | 12.77 | 4.00 |
| ATOM | 4373 | HG1 | GLU | 373 | 24.156 | 13.969 | 43.331 | 1.00 | 0.00 | 0.053 | 0.00 | 0.00 |
| ATOM | 4374 | HG2 | GLU | 373 | 24.283 | 15.582 | 43.962 | 1.00 | 0.00 | 0.053 | 0.00 | 0.00 |
| ATOM | 4375 | CD | GLU | 373 | 24.078 | 15.479 | 41.878 | 1.00 | 0.00 | 0.399 | 9.82 | 4.00 |
| ATOM | 4376 | OE1 | GLU | 373 | 23.253 | 16.206 | 41.271 | 1.00 | 0.00 | −0.396 | 8.17 | −18.95 |
| ATOM | 4377 | OE2 | GLU | 373 | 25.195 | 15.162 | 41.403 | 1.00 | 0.00 | −0.427 | 8.17 | −18.95 |
| ATOM | 4378 | HE2 | GLU | 373 | 25.305 | 15.586 | 40.471 | 1.00 | 0.00 | 0.424 | 0.00 | 0.00 |
| ATOM | 4379 | C | GLU | 373 | 21.694 | 12.621 | 42.823 | 1.00 | 0.00 | 0.396 | 9.82 | 4.00 |
| ATOM | 4380 | O | GLU | 373 | 22.504 | 12.052 | 42.102 | 1.00 | 0.00 | −0.396 | 8.17 | −17.40 |
| ATOM | 4381 | N | LYS | 374S | 21.075 | 12.003 | 43.827 | 1.00 | 0.00 | −0.650 | 9.00 | −17.40 |
| ATOM | 4382 | HN | LYS | 374S | 20.417 | 12.524 | 44.424 | 1.00 | 0.00 | 0.440 | 0.00 | 0.00 |
| ATOM | 4383 | CA | LYS | 374S | 21.325 | 10.599 | 44.081 | 1.00 | 0.00 | 0.158 | 9.40 | 4.00 |
| ATOM | 4384 | HA | LYS | 374S | 22.393 | 10.406 | 44.178 | 1.00 | 0.00 | 0.053 | 0.00 | 0.00 |
| ATOM | 4385 | CB | LYS | 374S | 20.704 | 10.195 | 45.419 | 1.00 | 0.00 | −0.106 | 12.77 | 4.00 |
| ATOM | 4386 | HB1 | LYS | 374S | 20.708 | 9.118 | 45.587 | 1.00 | 0.00 | 0.053 | 0.00 | 0.00 |
| ATOM | 4387 | HB2 | LYS | 374S | 19.662 | 10.500 | 45.519 | 1.00 | 0.00 | 0.053 | 0.00 | 0.00 |
| ATOM | 4388 | CG | LYS | 374S | 21.423 | 10.798 | 46.623 | 1.00 | 0.00 | −0.106 | 12.77 | 4.00 |
| ATOM | 4389 | HG1 | LYS | 374S | 21.406 | 11.886 | 46.576 | 1.00 | 0.00 | 0.053 | 0.00 | 0.00 |
| ATOM | 4390 | HG2 | LYS | 374S | 22.463 | 10.474 | 46.652 | 1.00 | 0.00 | 0.053 | 0.00 | 0.00 |
| ATOM | 4391 | CD | LYS | 374S | 20.758 | 10.370 | 47.913 | 1.00 | 0.00 | −0.106 | 12.77 | 4.00 |
| ATOM | 4392 | HD1 | LYS | 374S | 20.735 | 9.284 | 48.011 | 1.00 | 0.00 | 0.053 | 0.00 | 0.00 |
| ATOM | 4393 | HD2 | LYS | 374S | 19.727 | 10.721 | 47.969 | 1.00 | 0.00 | 0.053 | 0.00 | 0.00 |
| ATOM | 4394 | CE | LYS | 374S | 21.486 | 10.916 | 49.123 | 1.00 | 0.00 | 0.099 | 12.77 | 4.00 |
| ATOM | 4395 | HE1 | LYS | 374S | 21.540 | 12.003 | 49.075 | 1.00 | 0.00 | 0.053 | 0.00 | 0.00 |
| ATOM | 4396 | HE2 | LYS | 374S | 22.500 | 10.520 | 49.166 | 1.00 | 0.00 | 0.053 | 0.00 | 0.00 |
| ATOM | 4397 | NZ | LYS | 374S | 20.767 | 10.528 | 50.387 | 1.00 | 0.00 | −0.045 | 13.25 | −39.20 |
| ATOM | 4398 | HZ1 | LYS | 374S | 21.273 | 10.906 | 51.200 | 1.00 | 0.00 | 0.280 | 0.00 | 0.00 |
| ATOM | 4399 | HZ2 | LYS | 374S | 19.810 | 10.910 | 50.370 | 1.00 | 0.00 | 0.280 | 0.00 | 0.00 |
| ATOM | 4400 | HZ3 | LYS | 374S | 20.724 | 9.501 | 50.456 | 1.00 | 0.00 | 0.280 | 0.00 | 0.00 |
| ATOM | 4401 | C | LYS | 374S | 20.789 | 9.734 | 42.937 | 1.00 | 0.00 | 0.396 | 9.82 | 4.00 |
| ATOM | 4402 | O | LYS | 374S | 21.387 | 8.701 | 42.586 | 1.00 | 0.00 | −0.396 | 8.17 | −17.40 |
| ATOM | 4403 | N | VAL | 375 | 19.680 | 10.162 | 42.336 | 1.00 | 0.00 | −0.650 | 9.00 | −17.40 |
| ATOM | 4404 | HN | VAL | 375 | 19.236 | 11.034 | 42.658 | 1.00 | 0.00 | 0.440 | 0.00 | 0.00 |
| ATOM | 4405 | CA | VAL | 375 | 19.078 | 9.420 | 41.229 | 1.00 | 0.00 | 0.158 | 9.40 | 4.00 |
| ATOM | 4406 | HA | VAL | 375 | 18.978 | 8.371 | 41.509 | 1.00 | 0.00 | 0.053 | 0.00 | 0.00 |
| ATOM | 4407 | CB | VAL | 375 | 17.671 | 9.982 | 40.848 | 1.00 | 0.00 | −0.053 | 9.40 | 4.00 |
| ATOM | 4408 | HB | VAL | 375 | 17.743 | 11.046 | 40.626 | 1.00 | 0.00 | 0.053 | 0.00 | 0.00 |
| ATOM | 4409 | CG1 | VAL | 375 | 17.136 | 9.255 | 39.621 | 1.00 | 0.00 | −0.159 | 16.15 | 4.00 |
| ATOM | 4410 | HG1 | VAL | 375 | 16.154 | 9.652 | 39.361 | 1.00 | 0.00 | 0.053 | 0.00 | 0.00 |
| ATOM | 4411 | HG1 | VAL | 375 | 17.819 | 9.401 | 38.784 | 1.00 | 0.00 | 0.053 | 0.00 | 0.00 |
| ATOM | 4412 | HG1 | VAL | 375 | 17.050 | 8.190 | 39.837 | 1.00 | 0.00 | 0.053 | 0.00 | 0.00 |
| ATOM | 4413 | CG2 | VAL | 375 | 16.702 | 9.842 | 42.006 | 1.00 | 0.00 | −0.159 | 16.15 | 4.00 |
| ATOM | 4414 | HG2 | VAL | 375 | 15.729 | 10.240 | 41.716 | 1.00 | 0.00 | 0.053 | 0.00 | 0.00 |
| ATOM | 4415 | HG2 | VAL | 375 | 16.599 | 8.789 | 42.269 | 1.00 | 0.00 | 0.053 | 0.00 | 0.00 |
| ATOM | 4416 | HG2 | VAL | 375 | 17.079 | 10.395 | 42.865 | 1.00 | 0.00 | 0.053 | 0.00 | 0.00 |
| ATOM | 4417 | C | VAL | 375 | 19.973 | 9.538 | 39.995 | 1.00 | 0.00 | 0.396 | 9.82 | 4.00 |
| ATOM | 4418 | O | VAL | 375 | 20.294 | 8.547 | 39.345 | 1.00 | 0.00 | −0.396 | 8.17 | −17.40 |
| ATOM | 4419 | N | ARG | 376G | 20.355 | 10.775 | 39.685 | 1.00 | 0.00 | −0.650 | 9.00 | −17.40 |
| ATOM | 4420 | HN | ARG | 376G | 20.045 | 11.548 | 40.290 | 1.00 | 0.00 | 0.440 | 0.00 | 0.00 |
| ATOM | 4421 | CA | ARG | 376G | 21.188 | 11.089 | 38.534 | 1.00 | 0.00 | 0.158 | 9.40 | 4.00 |
| ATOM | 4422 | HA | ARG | 376G | 20.688 | 10.895 | 37.584 | 1.00 | 0.00 | 0.053 | 0.00 | 0.00 |
| ATOM | 4423 | CB | ARG | 376G | 21.509 | 12.597 | 38.512 | 1.00 | 0.00 | −0.106 | 12.77 | 4.00 |
| ATOM | 4424 | HB1 | ARG | 376G | 22.408 | 12.848 | 39.073 | 1.00 | 0.00 | 0.053 | 0.00 | 0.00 |
| ATOM | 4425 | HB2 | ARG | 376G | 20.713 | 13.207 | 38.938 | 1.00 | 0.00 | 0.053 | 0.00 | 0.00 |
| ATOM | 4426 | CG | ARG | 376G | 21.736 | 13.138 | 37.100 | 1.00 | 0.00 | −0.106 | 12.77 | 4.00 |
| ATOM | 4427 | HG1 | ARG | 376G | 20.785 | 13.512 | 36.720 | 1.00 | 0.00 | 0.053 | 0.00 | 0.00 |
| ATOM | 4428 | HG2 | ARG | 376G | 22.104 | 12.323 | 36.477 | 1.00 | 0.00 | 0.053 | 0.00 | 0.00 |
| ATOM | 4429 | CD | ARG | 376G | 22.754 | 14.285 | 37.017 | 1.00 | 0.00 | 0.374 | 12.77 | 4.00 |
| ATOM | 4430 | HD1 | ARG | 376G | 23.105 | 14.355 | 35.987 | 1.00 | 0.00 | 0.053 | 0.00 | 0.00 |
| ATOM | 4431 | HD2 | ARG | 376G | 23.580 | 14.057 | 37.690 | 1.00 | 0.00 | 0.053 | 0.00 | 0.00 |
| ATOM | 4432 | NE | ARG | 376G | 22.223 | 15.593 | 37.394 | 1.00 | 0.00 | −0.819 | 9.00 | −24.67 |
| ATOM | 4433 | HE | ARG | 376G | 21.336 | 15.624 | 37.918 | 1.00 | 0.00 | 0.407 | 0.00 | 0.00 |
| ATOM | 4434 | CZ | ARG | 376G | 22.821 | 16.749 | 37.098 | 1.00 | 0.00 | 0.796 | 6.95 | 4.00 |
| ATOM | 4435 | NH1 | ARG | 376G | 23.963 | 16.750 | 36.420 | 1.00 | 0.00 | −0.746 | 9.00 | −24.67 |
| ATOM | 4436 | HH1 | ARG | 376G | 24.425 | 17.641 | 36.191 | 1.00 | 0.00 | 0.407 | 0.00 | 0.00 |
| ATOM | 4437 | HH1 | ARG | 376G | 24.386 | 15.859 | 36.123 | 1.00 | 0.00 | 0.407 | 0.00 | 0.00 |
| ATOM | 4438 | NH2 | ARG | 376G | 22.290 | 17.906 | 37.484 | 1.00 | 0.00 | −0.746 | 9.00 | −24.67 |
| ATOM | 4439 | HH2 | ARG | 376G | 22.760 | 18.792 | 37.250 | 1.00 | 0.00 | 0.407 | 0.00 | 0.00 |
| ATOM | 4440 | HH2 | ARG | 376G | 21.408 | 17.915 | 38.017 | 1.00 | 0.00 | 0.407 | 0.00 | 0.00 |
| ATOM | 4441 | C | ARG | 376G | 22.487 | 10.275 | 38.499 | 1.00 | 0.00 | 0.396 | 9.82 | 4.00 |
| ATOM | 4442 | O | ARG | 376G | 22.866 | 9.747 | 37.452 | 1.00 | 0.00 | −0.396 | 8.17 | −17.40 |
| ATOM | 4443 | N | THR | 377 | 23.157 | 10.149 | 39.641 | 1.00 | 0.00 | −0.650 | 9.00 | −17.40 |
| ATOM | 4444 | HN | THR | 377 | 22.783 | 10.577 | 40.500 | 1.00 | 0.00 | 0.440 | 0.00 | 0.00 |
| ATOM | 4445 | CA | THR | 377 | 24.417 | 9.410 | 39.691 | 1.00 | 0.00 | 0.158 | 9.40 | 4.00 |
| ATOM | 4446 | HA | THR | 377 | 24.970 | 9.443 | 38.752 | 1.00 | 0.00 | 0.053 | 0.00 | 0.00 |
| ATOM | 4447 | CB | THR | 377 | 25.356 | 9.986 | 40.736 | 1.00 | 0.00 | 0.060 | 9.40 | 4.00 |
| ATOM | 4448 | HB | THR | 377 | 26.311 | 9.462 | 40.707 | 1.00 | 0.00 | 0.053 | 0.00 | 0.00 |
| ATOM | 4449 | OG1 | THR | 377 | 24.775 | 9.815 | 42.034 | 1.00 | 0.00 | −0.537 | 11.04 | −17.40 |
| ATOM | 4450 | HG1 | THR | 377 | 24.519 | 10.735 | 42.420 | 1.00 | 0.00 | 0.424 | 0.00 | 0.00 |
| ATOM | 4451 | CG2 | THR | 377 | 25.593 | 11.462 | 40.464 | 1.00 | 0.00 | −0.159 | 16.15 | 4.00 |
| ATOM | 4452 | HG2 | THR | 377 | 26.267 | 11.867 | 41.218 | 1.00 | 0.00 | 0.053 | 0.00 | 0.00 |
| ATOM | 4453 | HG2 | THR | 377 | 26.038 | 11.583 | 39.476 | 1.00 | 0.00 | 0.053 | 0.00 | 0.00 |
| ATOM | 4454 | HG2 | THR | 377 | 24.643 | 11.996 | 40.502 | 1.00 | 0.00 | 0.053 | 0.00 | 0.00 |
| ATOM | 4455 | C | THR | 377 | 24.232 | 7.942 | 40.002 | 1.00 | 0.00 | 0.396 | 9.82 | 4.00 |
| ATOM | 4456 | O | THR | 377 | 25.204 | 7.211 | 40.168 | 1.00 | 0.00 | −0.396 | 8.17 | −17.40 |
| ATOM | 4457 | N | ASN | 378 | 22.983 | 7.511 | 40.103 | 1.00 | 0.00 | −0.650 | 9.00 | −17.40 |
| ATOM | 4458 | HN | ASN | 378 | 22.206 | 8.178 | 39.990 | 1.00 | 0.00 | 0.440 | 0.00 | 0.00 |
| ATOM | 4459 | CA | ASN | 378 | 22.691 | 6.110 | 40.370 | 1.00 | 0.00 | 0.158 | 9.40 | 4.00 |
| ATOM | 4460 | HA | ASN | 378 | 21.627 | 5.875 | 40.318 | 1.00 | 0.00 | 0.053 | 0.00 | 0.00 |
| ATOM | 4461 | CB | ASN | 378 | 23.343 | 5.243 | 39.283 | 1.00 | 0.00 | −0.106 | 12.77 | 4.00 |
| ATOM | 4462 | HB1 | ASN | 378 | 24.249 | 4.744 | 39.625 | 1.00 | 0.00 | 0.053 | 0.00 | 0.00 |
| ATOM | 4463 | HB2 | ASN | 378 | 23.633 | 5.815 | 38.401 | 1.00 | 0.00 | 0.053 | 0.00 | 0.00 |
| ATOM | 4464 | CG | ASN | 378 | 22.428 | 4.140 | 38.779 | 1.00 | 0.00 | 0.396 | 9.82 | 4.00 |
| ATOM | 4465 | OD1 | ASN | 378 | 21.252 | 4.068 | 39.145 | 1.00 | 0.00 | −0.396 | 8.17 | −17.40 |
| ATOM | 4466 | ND2 | ASN | 378 | 22.966 | 3.276 | 37.924 | 1.00 | 0.00 | −0.879 | 13.25 | −17.40 |
| ATOM | 4467 | HD2 | ASN | 378 | 23.952 | 3.373 | 37.645 | 1.00 | 0.00 | 0.440 | 0.00 | 0.00 |
| ATOM | 4468 | HD2 | ASN | 378 | 22.396 | 2.508 | 37.540 | 1.00 | 0.00 | 0.440 | 0.00 | 0.00 |
| ATOM | 4469 | C | ASN | 378 | 23.144 | 5.653 | 41.763 | 1.00 | 0.00 | 0.396 | 9.82 | 4.00 |
| ATOM | 4470 | O | ASN | 378 | 23.641 | 4.534 | 41.931 | 1.00 | 0.00 | −0.396 | 8.17 | −17.40 |
| ATOM | 4471 | N | ASP | 379P | 22.984 | 6.513 | 42.764 | 1.00 | 0.00 | −0.650 | 9.00 | −17.40 |
| ATOM | 4472 | HN | ASP | 379P | 22.605 | 7.453 | 42.579 | 1.00 | 0.00 | 0.440 | 0.00 | 0.00 |
| ATOM | 4473 | CA | ASP | 379P | 23.344 | 6.126 | 44.123 | 1.00 | 0.00 | 0.158 | 9.40 | 4.00 |
| ATOM | 4474 | HA | ASP | 379P | 24.197 | 5.453 | 44.034 | 1.00 | 0.00 | 0.053 | 0.00 | 0.00 |
| ATOM | 4475 | CB | ASP | 379P | 23.711 | 7.358 | 44.956 | 1.00 | 0.00 | −0.336 | 12.77 | 4.00 |
| ATOM | 4476 | HB1 | ASP | 379P | 22.846 | 7.987 | 45.167 | 1.00 | 0.00 | 0.053 | 0.00 | 0.00 |
| ATOM | 4477 | HB2 | ASP | 379P | 24.441 | 7.994 | 44.455 | 1.00 | 0.00 | 0.053 | 0.00 | 0.00 |
| ATOM | 4478 | CG | ASP | 379P | 24.316 | 6.986 | 46.307 | 1.00 | 0.00 | 0.297 | 9.82 | 4.00 |
| ATOM | 4479 | OD1 | ASP | 379P | 24.511 | 5.780 | 46.570 | 1.00 | 0.00 | −0.534 | 8.17 | −18.95 |
| ATOM | 4480 | OD2 | ASP | 379P | 24.604 | 7.900 | 47.104 | 1.00 | 0.00 | −0.534 | 8.17 | −18.95 |
| ATOM | 4481 | C | ASP | 379P | 22.109 | 5.431 | 44.716 | 1.00 | 0.00 | 0.396 | 9.82 | 4.00 |
| ATOM | 4482 | O | ASP | 379P | 20.999 | 5.566 | 44.177 | 1.00 | 0.00 | −0.396 | 8.17 | −17.40 |
| ATOM | 4483 | N | ARG | 380G | 22.295 | 4.666 | 45.794 | 1.00 | 0.00 | −0.650 | 9.00 | −17.40 |
| ATOM | 4484 | HN | ARG | 380G | 23.243 | 4.558 | 46.180 | 1.00 | 0.00 | 0.440 | 0.00 | 0.00 |
| ATOM | 4485 | CA | ARG | 380G | 21.180 | 3.980 | 46.434 | 1.00 | 0.00 | 0.158 | 9.40 | 4.00 |
| ATOM | 4486 | HA | ARG | 380G | 21.492 | 3.310 | 47.235 | 1.00 | 0.00 | 0.053 | 0.00 | 0.00 |
| ATOM | 4487 | CB | ARG | 380G | 20.257 | 5.009 | 47.088 | 1.00 | 0.00 | −0.106 | 12.77 | 4.00 |
| ATOM | 4488 | HB1 | ARG | 380G | 19.383 | 4.484 | 47.475 | 1.00 | 0.00 | 0.053 | 0.00 | 0.00 |
| ATOM | 4489 | HB2 | ARG | 380G | 19.966 | 5.735 | 46.329 | 1.00 | 0.00 | 0.053 | 0.00 | 0.00 |
| ATOM | 4490 | CG | ARG | 380G | 20.880 | 5.783 | 48.250 | 1.00 | 0.00 | −0.106 | 12.77 | 4.00 |
| ATOM | 4491 | HG1 | ARG | 380G | 20.249 | 6.613 | 48.569 | 1.00 | 0.00 | 0.053 | 0.00 | 0.00 |
| ATOM | 4492 | HG2 | ARG | 380G | 21.849 | 6.203 | 47.984 | 1.00 | 0.00 | 0.053 | 0.00 | 0.00 |
| ATOM | 4493 | CD | ARG | 380G | 21.099 | 4.896 | 49.470 | 1.00 | 0.00 | 0.374 | 12.77 | 4.00 |
| ATOM | 4494 | HD1 | ARG | 380G | 21.609 | 5.419 | 50.278 | 1.00 | 0.00 | 0.053 | 0.00 | 0.00 |
| ATOM | 4495 | HD2 | ARG | 380G | 21.704 | 4.018 | 49.242 | 1.00 | 0.00 | 0.053 | 0.00 | 0.00 |
| ATOM | 4496 | NE | ARG | 380G | 19.842 | 4.397 | 50.030 | 1.00 | 0.00 | −0.819 | 9.00 | −24.67 |
| ATOM | 4497 | HE | ARG | 380G | 19.657 | 3.385 | 49.976 | 1.00 | 0.00 | 0.407 | 0.00 | 0.00 |
| ATOM | 4498 | CZ | ARG | 380G | 18.921 | 5.169 | 50.605 | 1.00 | 0.00 | 0.796 | 6.95 | 4.00 |
| ATOM | 4499 | NH1 | ARG | 380G | 19.120 | 6.478 | 50.698 | 1.00 | 0.00 | −0.746 | 9.00 | −24.67 |
| ATOM | 4500 | HH1 | ARG | 380G | 18.409 | 7.076 | 51.142 | 1.00 | 0.00 | 0.407 | 0.00 | 0.00 |
| ATOM | 4501 | HH1 | ARG | 380G | 19.985 | 6.895 | 50.326 | 1.00 | 0.00 | 0.407 | 0.00 | 0.00 |
| ATOM | 4502 | NH2 | ARG | 380G | 17.796 | 4.638 | 51.075 | 1.00 | 0.00 | −0.746 | 9.00 | −24.67 |
| ATOM | 4503 | HH2 | ARG | 380G | 17.088 | 5.240 | 51.518 | 1.00 | 0.00 | 0.407 | 0.00 | 0.00 |
| ATOM | 4504 | HH2 | ARG | 380G | 17.632 | 3.624 | 50.995 | 1.00 | 0.00 | 0.407 | 0.00 | 0.00 |
| ATOM | 4505 | C | ARG | 380G | 20.369 | 3.110 | 45.463 | 1.00 | 0.00 | 0.396 | 9.82 | 4.00 |
| ATOM | 4506 | O | ARG | 380G | 19.137 | 3.164 | 45.440 | 1.00 | 0.00 | −0.396 | 8.17 | −17.40 |
| ATOM | 4507 | N | LYS | 381S | 21.051 | 2.283 | 44.683 | 1.00 | 0.00 | −0.650 | 9.00 | −17.40 |
| ATOM | 4508 | HN | LYS | 381S | 22.076 | 2.232 | 44.761 | 1.00 | 0.00 | 0.440 | 0.00 | 0.00 |
| ATOM | 4509 | CA | LYS | 381S | 20.356 | 1.448 | 43.718 | 1.00 | 0.00 | 0.158 | 9.40 | 4.00 |
| ATOM | 4510 | HA | LYS | 381S | 19.782 | 2.101 | 43.061 | 1.00 | 0.00 | 0.053 | 0.00 | 0.00 |
| ATOM | 4511 | CB | LYS | 381S | 21.379 | 0.760 | 42.814 | 1.00 | 0.00 | −0.106 | 12.77 | 4.00 |
| ATOM | 4512 | HB1 | LYS | 381S | 20.912 | 0.032 | 42.150 | 1.00 | 0.00 | 0.053 | 0.00 | 0.00 |
| ATOM | 4513 | HB2 | LYS | 381S | 22.135 | 0.225 | 43.387 | 1.00 | 0.00 | 0.053 | 0.00 | 0.00 |
| ATOM | 4514 | CG | LYS | 381S | 22.113 | 1.749 | 41.934 | 1.00 | 0.00 | −0.106 | 12.77 | 4.00 |
| ATOM | 4515 | HG1 | LYS | 381S | 22.364 | 2.673 | 42.453 | 1.00 | 0.00 | 0.053 | 0.00 | 0.00 |
| ATOM | 4516 | HG2 | LYS | 381S | 21.536 | 2.046 | 41.058 | 1.00 | 0.00 | 0.053 | 0.00 | 0.00 |
| ATOM | 4517 | CD | LYS | 381S | 23.431 | 1.202 | 41.398 | 1.00 | 0.00 | −0.106 | 12.77 | 4.00 |
| ATOM | 4518 | HD1 | LYS | 381S | 24.080 | 0.829 | 42.190 | 1.00 | 0.00 | 0.053 | 0.00 | 0.00 |
| ATOM | 4519 | HD2 | LYS | 381S | 24.006 | 1.955 | 40.859 | 1.00 | 0.00 | 0.053 | 0.00 | 0.00 |
| ATOM | 4520 | CE | LYS | 381S | 23.231 | 0.053 | 40.437 | 1.00 | 0.00 | 0.099 | 12.77 | 4.00 |
| ATOM | 4521 | HE1 | LYS | 381S | 22.556 | 0.352 | 39.635 | 1.00 | 0.00 | 0.053 | 0.00 | 0.00 |
| ATOM | 4522 | HE2 | LYS | 381S | 22.801 | −0.800 | 40.962 | 1.00 | 0.00 | 0.053 | 0.00 | 0.00 |
| ATOM | 4523 | NZ | LYS | 381S | 24.545 | −0.351 | 39.843 | 1.00 | 0.00 | −0.045 | 13.25 | −39.20 |
| ATOM | 4524 | HZ1 | LYS | 381S | 24.400 | −1.134 | 39.189 | 1.00 | 0.00 | 0.280 | 0.00 | 0.00 |
| ATOM | 4525 | HZ2 | LYS | 381S | 24.953 | 0.447 | 39.336 | 1.00 | 0.00 | 0.280 | 0.00 | 0.00 |
| ATOM | 4526 | HZ3 | LYS | 381S | 25.186 | −0.644 | 40.594 | 1.00 | 0.00 | 0.280 | 0.00 | 0.00 |
| ATOM | 4527 | C | LYS | 381S | 19.380 | 0.428 | 44.299 | 1.00 | 0.00 | 0.396 | 9.82 | 4.00 |
| ATOM | 4528 | O | LYS | 381S | 18.582 | −0.145 | 43.561 | 1.00 | 0.00 | −0.396 | 8.17 | −17.40 |
| ATOM | 4529 | N | GLU | 382 | 19.410 | 0.201 | 45.611 | 1.00 | 0.00 | −0.650 | 9.00 | −17.40 |
| ATOM | 4530 | HN | GLU | 382 | 20.078 | 0.700 | 46.215 | 1.00 | 0.00 | 0.440 | 0.00 | 0.00 |
| ATOM | 4531 | CA | GLU | 382 | 18.480 | −0.766 | 46.175 | 1.00 | 0.00 | 0.158 | 9.40 | 4.00 |
| ATOM | 4532 | HA | GLU | 382 | 18.545 | −1.698 | 45.615 | 1.00 | 0.00 | 0.053 | 0.00 | 0.00 |
| ATOM | 4533 | CB | GLU | 382 | 18.808 | −1.102 | 47.643 | 1.00 | 0.00 | −0.106 | 12.77 | 4.00 |
| ATOM | 4534 | HB1 | GLU | 382 | 19.859 | −1.381 | 47.702 | 1.00 | 0.00 | 0.053 | 0.00 | 0.00 |
| ATOM | 4535 | HB2 | GLU | 382 | 18.171 | −1.930 | 47.953 | 1.00 | 0.00 | 0.053 | 0.00 | 0.00 |
| ATOM | 4536 | CG | GLU | 382 | 18.582 | 0.039 | 48.617 | 1.00 | 0.00 | −0.106 | 12.77 | 4.00 |
| ATOM | 4537 | HG1 | GLU | 382 | 18.378 | −0.312 | 49.628 | 1.00 | 0.00 | 0.053 | 0.00 | 0.00 |
| ATOM | 4538 | HG2 | GLU | 382 | 17.736 | 0.666 | 48.333 | 1.00 | 0.00 | 0.053 | 0.00 | 0.00 |
| ATOM | 4539 | CD | GLU | 382 | 19.775 | 0.953 | 48.717 | 1.00 | 0.00 | 0.399 | 9.82 | 4.00 |
| ATOM | 4540 | OE1 | GLU | 382 | 20.592 | 0.968 | 47.761 | 1.00 | 0.00 | −0.396 | 8.17 | −18.95 |
| ATOM | 4541 | OE2 | GLU | 382 | 19.891 | 1.659 | 49.746 | 1.00 | 0.00 | −0.427 | 8.17 | −18.95 |
| ATOM | 4542 | HE2 | GLU | 382 | 20.742 | 2.233 | 49.674 | 1.00 | 0.00 | 0.424 | 0.00 | 0.00 |
| ATOM | 4543 | C | GLU | 382 | 17.064 | −0.215 | 46.085 | 1.00 | 0.00 | 0.396 | 9.82 | 4.00 |
| ATOM | 4544 | O | GLU | 382 | 16.102 | −0.948 | 46.309 | 1.00 | 0.00 | −0.396 | 8.17 | −17.40 |
| ATOM | 4545 | N | LEU | 383 | 16.936 | 1.075 | 45.767 | 1.00 | 0.00 | −0.650 | 9.00 | −17.40 |
| ATOM | 4546 | HN | LEU | 383 | 17.780 | 1.641 | 45.603 | 1.00 | 0.00 | 0.440 | 0.00 | 0.00 |
| ATOM | 4547 | CA | LEU | 383 | 15.617 | 1.699 | 45.647 | 1.00 | 0.00 | 0.158 | 9.40 | 4.00 |
| ATOM | 4548 | HA | LEU | 383 | 14.934 | 1.110 | 46.260 | 1.00 | 0.00 | 0.053 | 0.00 | 0.00 |
| ATOM | 4549 | CB | LEU | 383 | 15.648 | 3.152 | 46.164 | 1.00 | 0.00 | −0.106 | 12.77 | 4.00 |
| ATOM | 4550 | HB1 | LEU | 383 | 14.634 | 3.551 | 46.116 | 1.00 | 0.00 | 0.053 | 0.00 | 0.00 |
| ATOM | 4551 | HB2 | LEU | 383 | 16.319 | 3.725 | 45.525 | 1.00 | 0.00 | 0.053 | 0.00 | 0.00 |
| ATOM | 4552 | CG | LEU | 383 | 16.137 | 3.312 | 47.605 | 1.00 | 0.00 | −0.053 | 9.40 | 4.00 |
| ATOM | 4553 | HG | LEU | 383 | 17.118 | 2.858 | 47.740 | 1.00 | 0.00 | 0.053 | 0.00 | 0.00 |
| ATOM | 4554 | CD1 | LEU | 383 | 16.250 | 4.789 | 47.990 | 1.00 | 0.00 | −0.159 | 16.15 | 4.00 |
| ATOM | 4555 | HD1 | LEU | 383 | 16.599 | 4.871 | 49.019 | 1.00 | 0.00 | 0.053 | 0.00 | 0.00 |
| ATOM | 4556 | HD1 | LEU | 383 | 16.957 | 5.285 | 47.325 | 1.00 | 0.00 | 0.053 | 0.00 | 0.00 |
| ATOM | 4557 | HD1 | LEU | 383 | 15.273 | 5.264 | 47.899 | 1.00 | 0.00 | 0.053 | 0.00 | 0.00 |
| ATOM | 4558 | CD2 | LEU | 383 | 15.169 | 2.568 | 48.541 | 1.00 | 0.00 | −0.159 | 16.15 | 4.00 |
| ATOM | 4559 | HD2 | LEU | 383 | 15.506 | 2.674 | 49.572 | 1.00 | 0.00 | 0.053 | 0.00 | 0.00 |
| ATOM | 4560 | HD2 | LEU | 383 | 14.169 | 2.990 | 48.441 | 1.00 | 0.00 | 0.053 | 0.00 | 0.00 |
| ATOM | 4561 | HD2 | LEU | 383 | 15.144 | 1.511 | 48.274 | 1.00 | 0.00 | 0.053 | 0.00 | 0.00 |
| ATOM | 4562 | C | LEU | 383 | 15.242 | 1.654 | 44.167 | 1.00 | 0.00 | 0.396 | 9.82 | 4.00 |
| ATOM | 4563 | O | LEU | 383 | 15.532 | 2.589 | 43.413 | 1.00 | 0.00 | −0.396 | 8.17 | −17.40 |
| ATOM | 4564 | N | GLY | 384 | 14.632 | 0.538 | 43.764 | 1.00 | 0.00 | −0.650 | 9.00 | −17.40 |
| ATOM | 4565 | HN | GLY | 384 | 14.446 | −0.205 | 44.452 | 1.00 | 0.00 | 0.440 | 0.00 | 0.00 |
| ATOM | 4566 | CA | GLY | 384 | 14.221 | 0.339 | 42.385 | 1.00 | 0.00 | 0.105 | 9.40 | 4.00 |
| ATOM | 4567 | HA1 | GLY | 384 | 13.898 | −0.696 | 42.276 | 1.00 | 0.00 | 0.053 | 0.00 | 0.00 |
| ATOM | 4568 | HA2 | GLY | 384 | 15.077 | 0.548 | 41.745 | 1.00 | 0.00 | 0.053 | 0.00 | 0.00 |
| ATOM | 4569 | C | GLY | 384 | 13.075 | 1.221 | 41.907 | 1.00 | 0.00 | 0.396 | 9.82 | 4.00 |
| ATOM | 4570 | O | GLY | 384 | 12.772 | 1.247 | 40.705 | 1.00 | 0.00 | −0.396 | 8.17 | −17.40 |
| ATOM | 4571 | N | GLU | 385 | 12.435 | 1.941 | 42.826 | 1.00 | 0.00 | −0.650 | 9.00 | −17.40 |
| ATOM | 4572 | HN | GLU | 385 | 12.725 | 1.873 | 43.812 | 1.00 | 0.00 | 0.440 | 0.00 | 0.00 |
| ATOM | 4573 | CA | GLU | 385 | 11.319 | 2.834 | 42.462 | 1.00 | 0.00 | 0.158 | 9.40 | 4.00 |
| ATOM | 4574 | HA | GLU | 385 | 11.482 | 3.193 | 41.446 | 1.00 | 0.00 | 0.053 | 0.00 | 0.00 |
| ATOM | 4575 | CB | GLU | 385 | 9.974 | 2.085 | 42.498 | 1.00 | 0.00 | −0.106 | 12.77 | 4.00 |
| ATOM | 4576 | HB1 | GLU | 385 | 9.813 | 1.716 | 43.511 | 1.00 | 0.00 | 0.053 | 0.00 | 0.00 |
| ATOM | 4577 | HB2 | GLU | 385 | 10.024 | 1.255 | 41.792 | 1.00 | 0.00 | 0.053 | 0.00 | 0.00 |
| ATOM | 4578 | CG | GLU | 385 | 8.762 | 2.949 | 42.118 | 1.00 | 0.00 | −0.106 | 12.77 | 4.00 |
| ATOM | 4579 | HG1 | GLU | 385 | 8.969 | 3.631 | 41.294 | 1.00 | 0.00 | 0.053 | 0.00 | 0.00 |
| ATOM | 4580 | HG2 | GLU | 385 | 8.411 | 3.573 | 42.940 | 1.00 | 0.00 | 0.053 | 0.00 | 0.00 |
| ATOM | 4581 | CD | GLU | 385 | 7.551 | 2.118 | 41.678 | 1.00 | 0.00 | 0.399 | 9.82 | 4.00 |
| ATOM | 4582 | OE1 | GLU | 385 | 7.681 | 0.883 | 41.577 | 1.00 | 0.00 | −0.396 | 8.17 | −18.95 |
| ATOM | 4583 | OE2 | GLU | 385 | 6.470 | 2.692 | 41.432 | 1.00 | 0.00 | −0.427 | 8.17 | −18.95 |
| ATOM | 4584 | HE2 | GLU | 385 | 5.766 | 1.994 | 41.152 | 1.00 | 0.00 | 0.424 | 0.00 | 0.00 |
| ATOM | 4585 | C | GLU | 385 | 11.296 | 3.995 | 43.439 | 1.00 | 0.00 | 0.396 | 9.82 | 4.00 |
| ATOM | 4586 | O | GLU | 385 | 11.085 | 3.814 | 44.646 | 1.00 | 0.00 | −0.396 | 8.17 | −17.40 |
| ATOM | 4587 | N | VAL | 386 | 11.519 | 5.195 | 42.924 | 1.00 | 0.00 | −0.650 | 9.00 | −17.40 |
| ATOM | 4588 | HN | VAL | 386 | 11.675 | 5.304 | 41.912 | 1.00 | 0.00 | 0.440 | 0.00 | 0.00 |
| ATOM | 4589 | CA | VAL | 386 | 11.541 | 6.355 | 43.797 | 1.00 | 0.00 | 0.158 | 9.40 | 4.00 |
| ATOM | 4590 | HA | VAL | 386 | 11.158 | 6.051 | 44.771 | 1.00 | 0.00 | 0.053 | 0.00 | 0.00 |
| ATOM | 4591 | CB | VAL | 386 | 12.957 | 6.868 | 43.990 | 1.00 | 0.00 | −0.053 | 9.40 | 4.00 |
| ATOM | 4592 | HB | VAL | 386 | 12.960 | 7.705 | 44.688 | 1.00 | 0.00 | 0.053 | 0.00 | 0.00 |
| ATOM | 4593 | CG1 | VAL | 386 | 13.844 | 5.761 | 44.546 | 1.00 | 0.00 | −0.159 | 16.15 | 4.00 |
| ATOM | 4594 | HG1 | VAL | 386 | 14.857 | 6.139 | 44.680 | 1.00 | 0.00 | 0.053 | 0.00 | 0.00 |
| ATOM | 4595 | HG1 | VAL | 386 | 13.450 | 5.427 | 45.506 | 1.00 | 0.00 | 0.053 | 0.00 | 0.00 |
| ATOM | 4596 | HG1 | VAL | 386 | 13.859 | 4.922 | 43.849 | 1.00 | 0.00 | 0.053 | 0.00 | 0.00 |
| ATOM | 4597 | CG2 | VAL | 386 | 13.500 | 7.407 | 42.657 | 1.00 | 0.00 | −0.159 | 16.15 | 4.00 |
| ATOM | 4598 | HG2 | VAL | 386 | 14.516 | 7.774 | 42.800 | 1.00 | 0.00 | 0.053 | 0.00 | 0.00 |
| ATOM | 4599 | HG2 | VAL | 386 | 13.503 | 6.607 | 41.916 | 1.00 | 0.00 | 0.053 | 0.00 | 0.00 |
| ATOM | 4600 | HG2 | VAL | 386 | 12.865 | 8.221 | 42.308 | 1.00 | 0.00 | 0.053 | 0.00 | 0.00 |
| ATOM | 4601 | C | VAL | 386 | 10.712 | 7.523 | 43.335 | 1.00 | 0.00 | 0.396 | 9.82 | 4.00 |
| ATOM | 4602 | O | VAL | 386 | 10.185 | 7.552 | 42.216 | 1.00 | 0.00 | −0.396 | 8.17 | −17.40 |
| ATOM | 4603 | N | ARG | 387G | 10.623 | 8.501 | 44.220 | 1.00 | 0.00 | −0.650 | 9.00 | −17.40 |
| ATOM | 4604 | HN | ARG | 387G | 11.084 | 8.380 | 45.133 | 1.00 | 0.00 | 0.440 | 0.00 | 0.00 |
| ATOM | 4605 | CA | ARG | 387G | 9.910 | 9.728 | 43.967 | 1.00 | 0.00 | 0.158 | 9.40 | 4.00 |
| ATOM | 4606 | HA | ARG | 387G | 9.530 | 9.648 | 42.948 | 1.00 | 0.00 | 0.053 | 0.00 | 0.00 |
| ATOM | 4607 | CB | ARG | 387G | 8.792 | 9.896 | 45.003 | 1.00 | 0.00 | −0.106 | 12.77 | 4.00 |
| ATOM | 4608 | HB1 | ARG | 387G | 9.258 | 10.049 | 45.976 | 1.00 | 0.00 | 0.053 | 0.00 | 0.00 |
| ATOM | 4609 | HB2 | ARG | 387G | 8.191 | 8.986 | 44.996 | 1.00 | 0.00 | 0.053 | 0.00 | 0.00 |
| ATOM | 4610 | CG | ARG | 387G | 7.845 | 11.053 | 44.780 | 1.00 | 0.00 | −0.106 | 12.77 | 4.00 |
| ATOM | 4611 | HG1 | ARG | 387G | 7.681 | 11.201 | 43.712 | 1.00 | 0.00 | 0.053 | 0.00 | 0.00 |
| ATOM | 4612 | HG2 | ARG | 387G | 8.264 | 11.965 | 45.203 | 1.00 | 0.00 | 0.053 | 0.00 | 0.00 |
| ATOM | 4613 | CD | ARG | 387G | 6.492 | 10.765 | 45.457 | 1.00 | 0.00 | 0.374 | 12.77 | 4.00 |
| ATOM | 4614 | HD1 | ARG | 387G | 5.863 | 10.113 | 44.850 | 1.00 | 0.00 | 0.053 | 0.00 | 0.00 |
| ATOM | 4615 | HD2 | ARG | 387G | 5.923 | 11.677 | 45.634 | 1.00 | 0.00 | 0.053 | 0.00 | 0.00 |
| ATOM | 4616 | NE | ARG | 387G | 6.659 | 10.107 | 46.756 | 1.00 | 0.00 | −0.819 | 9.00 | −24.67 |
| ATOM | 4617 | HE | ARG | 387G | 6.577 | 9.080 | 46.787 | 1.00 | 0.00 | 0.407 | 0.00 | 0.00 |
| ATOM | 4618 | CZ | ARG | 387G | 6.909 | 10.743 | 47.899 | 1.00 | 0.00 | 0.796 | 6.95 | 4.00 |
| ATOM | 4619 | NH1 | ARG | 387G | 7.014 | 12.061 | 47.913 | 1.00 | 0.00 | −0.746 | 9.00 | −24.67 |
| ATOM | 4620 | HH1 | ARG | 387G | 7.207 | 12.552 | 48.797 | 1.00 | 0.00 | 0.407 | 0.00 | 0.00 |
| ATOM | 4621 | HH1 | ARG | 387G | 6.902 | 12.595 | 47.039 | 1.00 | 0.00 | 0.407 | 0.00 | 0.00 |
| ATOM | 4622 | NH2 | ARG | 387G | 7.069 | 10.058 | 49.025 | 1.00 | 0.00 | −0.746 | 9.00 | −24.67 |
| ATOM | 4623 | HH2 | ARG | 387G | 7.262 | 10.554 | 49.906 | 1.00 | 0.00 | 0.407 | 0.00 | 0.00 |
| ATOM | 4624 | HH2 | ARG | 387G | 6.999 | 9.030 | 49.017 | 1.00 | 0.00 | 0.407 | 0.00 | 0.00 |
| ATOM | 4625 | C | ARG | 387G | 10.927 | 10.841 | 44.129 | 1.00 | 0.00 | 0.396 | 9.82 | 4.00 |
| ATOM | 4626 | O | ARG | 387G | 11.649 | 10.876 | 45.116 | 1.00 | 0.00 | −0.396 | 8.17 | −17.40 |
| ATOM | 4627 | N | VAL | 388 | 11.032 | 11.701 | 43.122 | 1.00 | 0.00 | −0.650 | 9.00 | −17.40 |
| ATOM | 4628 | HN | VAL | 388 | 10.474 | 11.537 | 42.271 | 1.00 | 0.00 | 0.440 | 0.00 | 0.00 |
| ATOM | 4629 | CA | VAL | 388 | 11.907 | 12.870 | 43.173 | 1.00 | 0.00 | 0.158 | 9.40 | 4.00 |
| ATOM | 4630 | HA | VAL | 388 | 12.586 | 12.838 | 44.024 | 1.00 | 0.00 | 0.053 | 0.00 | 0.00 |
| ATOM | 4631 | CB | VAL | 388 | 12.732 | 13.053 | 41.879 | 1.00 | 0.00 | −0.053 | 9.40 | 4.00 |
| ATOM | 4632 | HB | VAL | 388 | 12.084 | 12.966 | 41.006 | 1.00 | 0.00 | 0.053 | 0.00 | 0.00 |
| ATOM | 4633 | CG1 | VAL | 388 | 13.403 | 14.450 | 41.868 | 1.00 | 0.00 | −0.159 | 16.15 | 4.00 |
| ATOM | 4634 | HG1 | VAL | 388 | 13.981 | 14.568 | 40.952 | 1.00 | 0.00 | 0.053 | 0.00 | 0.00 |
| ATOM | 4635 | HG1 | VAL | 388 | 12.635 | 15.222 | 41.914 | 1.00 | 0.00 | 0.053 | 0.00 | 0.00 |
| ATOM | 4636 | HG1 | VAL | 388 | 14.064 | 14.542 | 42.729 | 1.00 | 0.00 | 0.053 | 0.00 | 0.00 |
| ATOM | 4637 | CG2 | VAL | 388 | 13.812 | 11.975 | 41.801 | 1.00 | 0.00 | −0.159 | 16.15 | 4.00 |
| ATOM | 4638 | HG2 | VAL | 388 | 14.392 | 12.106 | 40.887 | 1.00 | 0.00 | 0.053 | 0.00 | 0.00 |
| ATOM | 4639 | HG2 | VAL | 388 | 14.471 | 12.057 | 42.664 | 1.00 | 0.00 | 0.053 | 0.00 | 0.00 |
| ATOM | 4640 | HG2 | VAL | 388 | 13.343 | 10.990 | 41.795 | 1.00 | 0.00 | 0.053 | 0.00 | 0.00 |
| ATOM | 4641 | C | VAL | 388 | 10.842 | 13.954 | 43.284 | 1.00 | 0.00 | 0.396 | 9.82 | 4.00 |
| ATOM | 4642 | O | VAL | 388 | 10.032 | 14.146 | 42.366 | 1.00 | 0.00 | −0.396 | 8.17 | −17.40 |
| ATOM | 4643 | N | GLN | 389 | 10.811 | 14.636 | 44.422 | 1.00 | 0.00 | −0.650 | 9.00 | −17.40 |
| ATOM | 4644 | HN | GLN | 389 | 11.512 | 14.448 | 45.152 | 1.00 | 0.00 | 0.440 | 0.00 | 0.00 |
| ATOM | 4645 | CA | GLN | 389 | 9.782 | 15.654 | 44.642 | 1.00 | 0.00 | 0.158 | 9.40 | 4.00 |
| ATOM | 4646 | HA | GLN | 389 | 8.863 | 15.467 | 44.086 | 1.00 | 0.00 | 0.053 | 0.00 | 0.00 |
| ATOM | 4647 | CB | GLN | 389 | 9.365 | 15.639 | 46.117 | 1.00 | 0.00 | −0.106 | 12.77 | 4.00 |
| ATOM | 4648 | HB1 | GLN | 389 | 10.264 | 15.746 | 46.724 | 1.00 | 0.00 | 0.053 | 0.00 | 0.00 |
| ATOM | 4649 | HB2 | GLN | 389 | 8.872 | 14.688 | 46.319 | 1.00 | 0.00 | 0.053 | 0.00 | 0.00 |
| ATOM | 4650 | CG | GLN | 389 | 8.399 | 16.751 | 46.531 | 1.00 | 0.00 | −0.106 | 12.77 | 4.00 |
| ATOM | 4651 | HG1 | GLN | 389 | 7.521 | 16.798 | 45.886 | 1.00 | 0.00 | 0.053 | 0.00 | 0.00 |
| ATOM | 4652 | HG2 | GLN | 389 | 8.862 | 17.736 | 46.494 | 1.00 | 0.00 | 0.053 | 0.00 | 0.00 |
| ATOM | 4653 | CD | GLN | 389 | 7.879 | 16.568 | 47.960 | 1.00 | 0.00 | 0.396 | 9.82 | 4.00 |
| ATOM | 4654 | OE1 | GLN | 389 | 7.020 | 15.722 | 48.217 | 1.00 | 0.00 | −0.396 | 8.17 | −17.40 |
| ATOM | 4655 | NE2 | GLN | 389 | 8.410 | 17.356 | 48.894 | 1.00 | 0.00 | −0.879 | 13.25 | −17.40 |
| ATOM | 4656 | HE2 | GLN | 389 | 9.125 | 18.050 | 48.636 | 1.00 | 0.00 | 0.440 | 0.00 | 0.00 |
| ATOM | 4657 | HE2 | GLN | 389 | 8.104 | 17.270 | 49.874 | 1.00 | 0.00 | 0.440 | 0.00 | 0.00 |
| ATOM | 4658 | C | GLN | 389 | 10.218 | 17.047 | 44.237 | 1.00 | 0.00 | 0.396 | 9.82 | 4.00 |
| ATOM | 4659 | O | GLN | 389 | 11.326 | 17.470 | 44.561 | 1.00 | 0.00 | −0.396 | 8.17 | −17.40 |
| ATOM | 4660 | N | TYR | 390 | 9.360 | 17.745 | 43.493 | 1.00 | 0.00 | −0.650 | 9.00 | −17.40 |
| ATOM | 4661 | HN | TYR | 390 | 8.474 | 17.307 | 43.200 | 1.00 | 0.00 | 0.440 | 0.00 | 0.00 |
| ATOM | 4662 | CA | TYR | 390 | 9.650 | 19.118 | 43.084 | 1.00 | 0.00 | 0.158 | 9.40 | 4.00 |
| ATOM | 4663 | HA | TYR | 390 | 10.666 | 19.302 | 43.431 | 1.00 | 0.00 | 0.053 | 0.00 | 0.00 |
| ATOM | 4664 | CB | TYR | 390 | 9.560 | 19.282 | 41.555 | 1.00 | 0.00 | −0.106 | 12.77 | 4.00 |
| ATOM | 4665 | HB1 | TYR | 390 | 10.225 | 18.549 | 41.098 | 1.00 | 0.00 | 0.053 | 0.00 | 0.00 |
| ATOM | 4666 | HB2 | TYR | 390 | 9.869 | 20.297 | 41.306 | 1.00 | 0.00 | 0.053 | 0.00 | 0.00 |
| ATOM | 4667 | CG | TYR | 390 | 8.179 | 19.072 | 40.953 | 1.00 | 0.00 | 0.000 | 7.26 | 0.60 |
| ATOM | 4668 | CD1 | TYR | 390 | 7.216 | 20.104 | 40.964 | 1.00 | 0.00 | −0.127 | 10.80 | 0.60 |
| ATOM | 4669 | HD1 | TYR | 390 | 7.449 | 21.045 | 41.461 | 1.00 | 0.00 | 0.127 | 0.00 | 0.00 |
| ATOM | 4670 | CE1 | TYR | 390 | 5.970 | 19.946 | 40.352 | 1.00 | 0.00 | −0.127 | 10.80 | 0.60 |
| ATOM | 4671 | HE1 | TYR | 390 | 5.241 | 20.756 | 40.369 | 1.00 | 0.00 | 0.127 | 0.00 | 0.00 |
| ATOM | 4672 | CD2 | TYR | 390 | 7.852 | 17.872 | 40.324 | 1.00 | 0.00 | −0.127 | 10.80 | 0.60 |
| ATOM | 4673 | HD2 | TYR | 390 | 8.576 | 17.058 | 40.312 | 1.00 | 0.00 | 0.127 | 0.00 | 0.00 |
| ATOM | 4674 | CE2 | TYR | 390 | 6.599 | 17.694 | 39.703 | 1.00 | 0.00 | −0.127 | 10.80 | 0.60 |
| ATOM | 4675 | HE2 | TYR | 390 | 6.358 | 16.749 | 39.215 | 1.00 | 0.00 | 0.127 | 0.00 | 0.00 |
| ATOM | 4676 | CZ | TYR | 390 | 5.668 | 18.734 | 39.716 | 1.00 | 0.00 | 0.026 | 7.26 | 0.60 |
| ATOM | 4677 | OH | TYR | 390 | 4.460 | 18.569 | 39.061 | 1.00 | 0.00 | −0.451 | 10.94 | −17.40 |
| ATOM | 4678 | HH | TYR | 390 | 4.044 | 19.491 | 38.869 | 1.00 | 0.00 | 0.424 | 0.00 | 0.00 |
| ATOM | 4679 | C | TYR | 390 | 8.595 | 19.966 | 43.792 | 1.00 | 0.00 | 0.396 | 9.82 | 4.00 |
| ATOM | 4680 | O | TYR | 390 | 7.483 | 19.495 | 44.058 | 1.00 | 0.00 | −0.396 | 8.17 | −17.40 |
| ATOM | 4681 | N | THR | 391 | 8.932 | 21.203 | 44.116 | 1.00 | 0.00 | −0.650 | 9.00 | −17.40 |
| ATOM | 4682 | HN | THR | 391 | 9.862 | 21.569 | 43.870 | 1.00 | 0.00 | 0.440 | 0.00 | 0.00 |
| ATOM | 4683 | CA | THR | 391 | 7.976 | 22.041 | 44.822 | 1.00 | 0.00 | 0.158 | 9.40 | 4.00 |
| ATOM | 4684 | HA | THR | 391 | 7.068 | 21.460 | 44.986 | 1.00 | 0.00 | 0.053 | 0.00 | 0.00 |
| ATOM | 4685 | CB | THR | 391 | 8.522 | 22.430 | 46.210 | 1.00 | 0.00 | 0.060 | 9.40 | 4.00 |
| ATOM | 4686 | HB | THR | 391 | 7.878 | 23.179 | 46.670 | 1.00 | 0.00 | 0.053 | 0.00 | 0.00 |
| ATOM | 4687 | OG1 | THR | 391 | 9.841 | 22.964 | 46.061 | 1.00 | 0.00 | −0.537 | 11.04 | −17.40 |
| ATOM | 4688 | HG1 | THR | 391 | 10.520 | 22.332 | 46.508 | 1.00 | 0.00 | 0.424 | 0.00 | 0.00 |
| ATOM | 4689 | CG2 | THR | 391 | 8.575 | 21.211 | 47.112 | 1.00 | 0.00 | −0.159 | 16.15 | 4.00 |
| ATOM | 4690 | HG2 | THR | 391 | 8.962 | 21.498 | 48.089 | 1.00 | 0.00 | 0.053 | 0.00 | 0.00 |
| ATOM | 4691 | HG2 | THR | 391 | 7.572 | 20.798 | 47.225 | 1.00 | 0.00 | 0.053 | 0.00 | 0.00 |
| ATOM | 4692 | HG2 | THR | 391 | 9.228 | 20.459 | 46.669 | 1.00 | 0.00 | 0.053 | 0.00 | 0.00 |
| ATOM | 4693 | C | THR | 391 | 7.586 | 23.304 | 44.082 | 1.00 | 0.00 | 0.396 | 9.82 | 4.00 |
| ATOM | 4694 | O | THR | 391 | 6.542 | 23.880 | 44.348 | 1.00 | 0.00 | −0.396 | 8.17 | −17.40 |
| ATOM | 4695 | N | GLY | 392 | 8.421 | 23.752 | 43.161 | 1.00 | 0.00 | −0.650 | 9.00 | −17.40 |
| ATOM | 4696 | HN | GLY | 392 | 9.300 | 23.253 | 42.964 | 1.00 | 0.00 | 0.440 | 0.00 | 0.00 |
| ATOM | 4697 | CA | GLY | 392 | 8.078 | 24.956 | 42.434 | 1.00 | 0.00 | 0.105 | 9.40 | 4.00 |
| ATOM | 4698 | HA1 | GLY | 392 | 8.658 | 25.800 | 42.805 | 1.00 | 0.00 | 0.053 | 0.00 | 0.00 |
| ATOM | 4699 | HA2 | GLY | 392 | 7.018 | 25.182 | 42.554 | 1.00 | 0.00 | 0.053 | 0.00 | 0.00 |
| ATOM | 4700 | C | GLY | 392 | 8.368 | 24.782 | 40.968 | 1.00 | 0.00 | 0.396 | 9.82 | 4.00 |
| ATOM | 4701 | O | GLY | 392 | 8.803 | 23.719 | 40.542 | 1.00 | 0.00 | −0.396 | 8.17 | −17.40 |
| ATOM | 4702 | N | ARG | 393G | 8.131 | 25.829 | 40.196 | 1.00 | 0.00 | −0.650 | 9.00 | −17.40 |
| ATOM | 4703 | HN | ARG | 393G | 7.761 | 26.692 | 40.618 | 1.00 | 0.00 | 0.440 | 0.00 | 0.00 |
| ATOM | 4704 | CA | ARG | 393G | 8.380 | 25.788 | 38.764 | 1.00 | 0.00 | 0.158 | 9.40 | 4.00 |
| ATOM | 4705 | HA | ARG | 393G | 7.863 | 24.954 | 38.289 | 1.00 | 0.00 | 0.053 | 0.00 | 0.00 |
| ATOM | 4706 | CB | ARG | 393G | 7.820 | 27.057 | 38.108 | 1.00 | 0.00 | −0.106 | 12.77 | 4.00 |
| ATOM | 4707 | HB1 | ARG | 393G | 6.731 | 27.008 | 38.142 | 1.00 | 0.00 | 0.053 | 0.00 | 0.00 |
| ATOM | 4708 | HB2 | ARG | 393G | 8.170 | 27.092 | 37.076 | 1.00 | 0.00 | 0.053 | 0.00 | 0.00 |
| ATOM | 4709 | CG | ARG | 393G | 8.249 | 28.363 | 38.790 | 1.00 | 0.00 | −0.106 | 12.77 | 4.00 |
| ATOM | 4710 | HG1 | ARG | 393G | 9.310 | 28.565 | 38.644 | 1.00 | 0.00 | 0.053 | 0.00 | 0.00 |
| ATOM | 4711 | HG2 | ARG | 393G | 8.073 | 28.334 | 39.865 | 1.00 | 0.00 | 0.053 | 0.00 | 0.00 |
| ATOM | 4712 | CD | ARG | 393G | 7.482 | 29.569 | 38.241 | 1.00 | 0.00 | 0.374 | 12.77 | 4.00 |
| ATOM | 4713 | HD1 | ARG | 393G | 7.335 | 29.524 | 37.162 | 1.00 | 0.00 | 0.053 | 0.00 | 0.00 |
| ATOM | 4714 | HD2 | ARG | 393G | 7.985 | 30.516 | 38.432 | 1.00 | 0.00 | 0.053 | 0.00 | 0.00 |
| ATOM | 4715 | NE | ARG | 393G | 6.144 | 29.706 | 38.819 | 1.00 | 0.00 | −0.819 | 9.00 | −24.67 |
| ATOM | 4716 | HE | ARG | 393G | 5.980 | 29.319 | 39.759 | 1.00 | 0.00 | 0.407 | 0.00 | 0.00 |
| ATOM | 4717 | CZ | ARG | 393G | 5.125 | 30.302 | 38.200 | 1.00 | 0.00 | 0.796 | 6.95 | 4.00 |
| ATOM | 4718 | NH1 | ARG | 393G | 5.306 | 30.805 | 36.988 | 1.00 | 0.00 | −0.746 | 9.00 | −24.67 |
| ATOM | 4719 | HH1 | ARG | 393G | 4.525 | 31.267 | 36.500 | 1.00 | 0.00 | 0.407 | 0.00 | 0.00 |
| ATOM | 4720 | HH1 | ARG | 393G | 6.227 | 30.733 | 36.533 | 1.00 | 0.00 | 0.407 | 0.00 | 0.00 |
| ATOM | 4721 | NH2 | ARG | 393G | 3.929 | 30.394 | 38.782 | 1.00 | 0.00 | −0.746 | 9.00 | −24.67 |
| ATOM | 4722 | HH2 | ARG | 393G | 3.151 | 30.857 | 38.290 | 1.00 | 0.00 | 0.407 | 0.00 | 0.00 |
| ATOM | 4723 | HH2 | ARG | 393G | 3.782 | 30.001 | 39.723 | 1.00 | 0.00 | 0.407 | 0.00 | 0.00 |
| ATOM | 4724 | C | ARG | 393G | 9.868 | 25.632 | 38.454 | 1.00 | 0.00 | 0.396 | 9.82 | 4.00 |
| ATOM | 4725 | O | ARG | 393G | 10.243 | 24.902 | 37.544 | 1.00 | 0.00 | −0.396 | 8.17 | −17.40 |
| ATOM | 4726 | N | ASP | 394P | 10.715 | 26.312 | 39.215 | 1.00 | 0.00 | −0.650 | 9.00 | −17.40 |
| ATOM | 4727 | HN | ASP | 394P | 10.349 | 26.909 | 39.970 | 1.00 | 0.00 | 0.440 | 0.00 | 0.00 |
| ATOM | 4728 | CA | ASP | 394P | 12.158 | 26.228 | 39.001 | 1.00 | 0.00 | 0.158 | 9.40 | 4.00 |
| ATOM | 4729 | HA | ASP | 394P | 12.442 | 26.445 | 37.971 | 1.00 | 0.00 | 0.053 | 0.00 | 0.00 |
| ATOM | 4730 | CB | ASP | 394P | 12.883 | 27.277 | 39.851 | 1.00 | 0.00 | −0.336 | 12.77 | 4.00 |
| ATOM | 4731 | HB1 | ASP | 394P | 13.966 | 27.213 | 39.755 | 1.00 | 0.00 | 0.053 | 0.00 | 0.00 |
| ATOM | 4732 | HB2 | ASP | 394P | 12.662 | 27.175 | 40.913 | 1.00 | 0.00 | 0.053 | 0.00 | 0.00 |
| ATOM | 4733 | CG | ASP | 394P | 12.503 | 28.694 | 39.469 | 1.00 | 0.00 | 0.297 | 9.82 | 4.00 |
| ATOM | 4734 | OD1 | ASP | 394P | 12.436 | 28.973 | 38.249 | 1.00 | 0.00 | −0.534 | 8.17 | −18.95 |
| ATOM | 4735 | OD2 | ASP | 394P | 12.274 | 29.526 | 40.379 | 1.00 | 0.00 | −0.534 | 8.17 | −18.95 |
| ATOM | 4736 | C | ASP | 394P | 12.720 | 24.838 | 39.313 | 1.00 | 0.00 | 0.396 | 9.82 | 4.00 |
| ATOM | 4737 | O | ASP | 394P | 13.574 | 24.331 | 38.573 | 1.00 | 0.00 | −0.396 | 8.17 | −17.40 |
| ATOM | 4738 | N | SER | 395 | 12.260 | 24.231 | 40.409 | 1.00 | 0.00 | −0.650 | 9.00 | −17.40 |
| ATOM | 4739 | HN | SER | 395 | 11.569 | 24.708 | 41.006 | 1.00 | 0.00 | 0.440 | 0.00 | 0.00 |
| ATOM | 4740 | CA | SER | 395 | 12.727 | 22.900 | 40.767 | 1.00 | 0.00 | 0.158 | 9.40 | 4.00 |
| ATOM | 4741 | HA | SER | 395 | 13.816 | 22.888 | 40.773 | 1.00 | 0.00 | 0.053 | 0.00 | 0.00 |
| ATOM | 4742 | CB | SER | 395 | 12.289 | 22.512 | 42.194 | 1.00 | 0.00 | 0.007 | 12.77 | 4.00 |
| ATOM | 4743 | HB1 | SER | 395 | 12.743 | 23.133 | 42.965 | 1.00 | 0.00 | 0.053 | 0.00 | 0.00 |
| ATOM | 4744 | HB2 | SER | 395 | 12.544 | 21.485 | 42.456 | 1.00 | 0.00 | 0.053 | 0.00 | 0.00 |
| ATOM | 4745 | OG | SER | 395 | 10.883 | 22.619 | 42.397 | 1.00 | 0.00 | −0.537 | 11.04 | −17.40 |
| ATOM | 4746 | HG | SER | 395 | 10.616 | 23.613 | 42.440 | 1.00 | 0.00 | 0.424 | 0.00 | 0.00 |
| ATOM | 4747 | C | SER | 395 | 12.208 | 21.884 | 39.740 | 1.00 | 0.00 | 0.396 | 9.82 | 4.00 |
| ATOM | 4748 | O | SER | 395 | 12.932 | 20.954 | 39.351 | 1.00 | 0.00 | −0.396 | 8.17 | −17.40 |
| ATOM | 4749 | N | PHE | 396 | 10.964 | 22.054 | 39.290 | 1.00 | 0.00 | −0.650 | 9.00 | −17.40 |
| ATOM | 4750 | HN | PHE | 396 | 10.380 | 22.827 | 39.640 | 1.00 | 0.00 | 0.440 | 0.00 | 0.00 |
| ATOM | 4751 | CA | PHE | 396 | 10.444 | 21.123 | 38.291 | 1.00 | 0.00 | 0.158 | 9.40 | 4.00 |
| ATOM | 4752 | HA | PHE | 396 | 10.405 | 20.111 | 38.696 | 1.00 | 0.00 | 0.053 | 0.00 | 0.00 |
| ATOM | 4753 | CB | PHE | 396 | 9.024 | 21.482 | 37.830 | 1.00 | 0.00 | −0.106 | 12.77 | 4.00 |
| ATOM | 4754 | HB1 | PHE | 396 | 8.918 | 22.533 | 37.564 | 1.00 | 0.00 | 0.053 | 0.00 | 0.00 |
| ATOM | 4755 | HB2 | PHE | 396 | 8.270 | 21.289 | 38.593 | 1.00 | 0.00 | 0.053 | 0.00 | 0.00 |
| ATOM | 4756 | CG | PHE | 396 | 8.589 | 20.703 | 36.614 | 1.00 | 0.00 | 0.000 | 7.26 | 0.60 |
| ATOM | 4757 | CD1 | PHE | 396 | 8.242 | 19.354 | 36.720 | 1.00 | 0.00 | −0.127 | 10.80 | 0.60 |
| ATOM | 4758 | HD1 | PHE | 396 | 8.192 | 18.888 | 37.704 | 1.00 | 0.00 | 0.127 | 0.00 | 0.00 |
| ATOM | 4759 | CD2 | PHE | 396 | 8.635 | 21.282 | 35.345 | 1.00 | 0.00 | −0.127 | 10.80 | 0.60 |
| ATOM | 4760 | HD2 | PHE | 396 | 8.894 | 22.335 | 35.243 | 1.00 | 0.00 | 0.127 | 0.00 | 0.00 |
| ATOM | 4761 | CE1 | PHE | 396 | 7.959 | 18.595 | 35.588 | 1.00 | 0.00 | −0.127 | 10.80 | 0.60 |
| ATOM | 4762 | HE1 | PHE | 396 | 7.692 | 17.542 | 35.688 | 1.00 | 0.00 | 0.127 | 0.00 | 0.00 |
| ATOM | 4763 | CE2 | PHE | 396 | 8.354 | 20.530 | 34.209 | 1.00 | 0.00 | −0.127 | 10.80 | 0.60 |
| ATOM | 4764 | HE2 | PHE | 396 | 8.397 | 20.994 | 33.224 | 1.00 | 0.00 | 0.127 | 0.00 | 0.00 |
| ATOM | 4765 | CZ | PHE | 396 | 8.017 | 19.181 | 34.330 | 1.00 | 0.00 | −0.127 | 10.80 | 0.60 |
| ATOM | 4766 | HZ | PHE | 396 | 7.800 | 18.589 | 33.440 | 1.00 | 0.00 | 0.127 | 0.00 | 0.00 |
| ATOM | 4767 | C | PHE | 396 | 11.335 | 21.114 | 37.058 | 1.00 | 0.00 | 0.396 | 9.82 | 4.00 |
| ATOM | 4768 | O | PHE | 396 | 11.832 | 20.067 | 36.649 | 1.00 | 0.00 | −0.396 | 8.17 | −17.40 |
| ATOM | 4769 | N | LYS | 397S | 11.539 | 22.278 | 36.451 | 1.00 | 0.00 | −0.650 | 9.00 | −17.40 |
| ATOM | 4770 | HN | LYS | 397S | 11.119 | 23.138 | 36.830 | 1.00 | 0.00 | 0.440 | 0.00 | 0.00 |
| ATOM | 4771 | CA | LYS | 397S | 12.359 | 22.333 | 35.245 | 1.00 | 0.00 | 0.158 | 9.40 | 4.00 |
| ATOM | 4772 | HA | LYS | 397S | 11.994 | 21.699 | 34.437 | 1.00 | 0.00 | 0.053 | 0.00 | 0.00 |
| ATOM | 4773 | CB | LYS | 397S | 12.317 | 23.733 | 34.614 | 1.00 | 0.00 | −0.106 | 12.77 | 4.00 |
| ATOM | 4774 | HB1 | LYS | 397S | 13.281 | 24.049 | 34.216 | 1.00 | 0.00 | 0.053 | 0.00 | 0.00 |
| ATOM | 4775 | HB2 | LYS | 397S | 12.018 | 24.508 | 35.319 | 1.00 | 0.00 | 0.053 | 0.00 | 0.00 |
| ATOM | 4776 | CG | LYS | 397S | 11.326 | 23.833 | 33.437 | 1.00 | 0.00 | −0.106 | 12.77 | 4.00 |
| ATOM | 4777 | HG1 | LYS | 397S | 10.289 | 23.721 | 33.756 | 1.00 | 0.00 | 0.053 | 0.00 | 0.00 |
| ATOM | 4778 | HG2 | LYS | 397S | 11.499 | 23.066 | 32.682 | 1.00 | 0.00 | 0.053 | 0.00 | 0.00 |
| ATOM | 4779 | CD | LYS | 397S | 11.413 | 25.167 | 32.721 | 1.00 | 0.00 | −0.106 | 12.77 | 4.00 |
| ATOM | 4780 | HD1 | LYS | 397S | 12.429 | 25.388 | 32.395 | 1.00 | 0.00 | 0.053 | 0.00 | 0.00 |
| ATOM | 4781 | HD2 | LYS | 397S | 11.097 | 25.992 | 33.359 | 1.00 | 0.00 | 0.053 | 0.00 | 0.00 |
| ATOM | 4782 | CE | LYS | 397S | 10.528 | 25.204 | 31.474 | 1.00 | 0.00 | 0.099 | 12.77 | 4.00 |
| ATOM | 4783 | HE1 | LYS | 397S | 10.468 | 26.235 | 31.127 | 1.00 | 0.00 | 0.053 | 0.00 | 0.00 |
| ATOM | 4784 | HE2 | LYS | 397S | 9.539 | 24.832 | 31.744 | 1.00 | 0.00 | 0.053 | 0.00 | 0.00 |
| ATOM | 4785 | NZ | LYS | 397S | 11.032 | 24.371 | 30.339 | 1.00 | 0.00 | −0.045 | 13.25 | −39.20 |
| ATOM | 4786 | HZ1 | LYS | 397S | 10.384 | 24.448 | 29.541 | 1.00 | 0.00 | 0.280 | 0.00 | 0.00 |
| ATOM | 4787 | HZ2 | LYS | 397S | 11.965 | 24.703 | 30.056 | 1.00 | 0.00 | 0.280 | 0.00 | 0.00 |
| ATOM | 4788 | HZ3 | LYS | 397S | 11.093 | 23.386 | 30.636 | 1.00 | 0.00 | 0.280 | 0.00 | 0.00 |
| ATOM | 4789 | C | LYS | 397S | 13.803 | 21.896 | 35.464 | 1.00 | 0.00 | 0.396 | 9.82 | 4.00 |
| ATOM | 4790 | O | LYS | 397S | 14.410 | 21.302 | 34.577 | 1.00 | 0.00 | −0.396 | 8.17 | −17.40 |
| ATOM | 4791 | N | ALA | 398 | 14.355 | 22.178 | 36.639 | 1.00 | 0.00 | −0.650 | 9.00 | −17.40 |
| ATOM | 4792 | HN | ALA | 398 | 13.810 | 22.681 | 37.354 | 1.00 | 0.00 | 0.440 | 0.00 | 0.00 |
| ATOM | 4793 | CA | ALA | 398 | 15.736 | 21.775 | 36.921 | 1.00 | 0.00 | 0.158 | 9.40 | 4.00 |
| ATOM | 4794 | HA | ALA | 398 | 16.397 | 22.102 | 36.119 | 1.00 | 0.00 | 0.053 | 0.00 | 0.00 |
| ATOM | 4795 | CB | ALA | 398 | 16.221 | 22.427 | 38.194 | 1.00 | 0.00 | −0.159 | 16.15 | 4.00 |
| ATOM | 4796 | HB1 | ALA | 398 | 17.247 | 22.119 | 38.392 | 1.00 | 0.00 | 0.053 | 0.00 | 0.00 |
| ATOM | 4797 | HB2 | ALA | 398 | 16.181 | 23.510 | 38.085 | 1.00 | 0.00 | 0.053 | 0.00 | 0.00 |
| ATOM | 4798 | HB3 | ALA | 398 | 15.584 | 22.121 | 39.024 | 1.00 | 0.00 | 0.053 | 0.00 | 0.00 |
| ATOM | 4799 | C | ALA | 398 | 15.832 | 20.252 | 37.041 | 1.00 | 0.00 | 0.396 | 9.82 | 4.00 |
| ATOM | 4800 | O | ALA | 398 | 16.681 | 19.627 | 36.412 | 1.00 | 0.00 | −0.396 | 8.17 | −17.40 |
| ATOM | 4801 | N | PHE | 399 | 14.954 | 19.658 | 37.846 | 1.00 | 0.00 | −0.650 | 9.00 | −17.40 |
| ATOM | 4802 | HN | PHE | 399 | 14.258 | 20.228 | 38.347 | 1.00 | 0.00 | 0.440 | 0.00 | 0.00 |
| ATOM | 4803 | CA | PHE | 399 | 14.963 | 18.210 | 38.029 | 1.00 | 0.00 | 0.158 | 9.40 | 4.00 |
| ATOM | 4804 | HA | PHE | 399 | 15.946 | 17.870 | 38.355 | 1.00 | 0.00 | 0.053 | 0.00 | 0.00 |
| ATOM | 4805 | CB | PHE | 399 | 13.982 | 17.806 | 39.132 | 1.00 | 0.00 | −0.106 | 12.77 | 4.00 |
| ATOM | 4806 | HB1 | PHE | 399 | 13.999 | 16.720 | 39.226 | 1.00 | 0.00 | 0.053 | 0.00 | 0.00 |
| ATOM | 4807 | HB2 | PHE | 399 | 12.986 | 18.149 | 38.849 | 1.00 | 0.00 | 0.053 | 0.00 | 0.00 |
| ATOM | 4808 | CG | PHE | 399 | 14.305 | 18.391 | 40.484 | 1.00 | 0.00 | 0.000 | 7.26 | 0.60 |
| ATOM | 4809 | CD1 | PHE | 399 | 13.472 | 18.153 | 41.565 | 1.00 | 0.00 | −0.127 | 10.80 | 0.60 |
| ATOM | 4810 | HD1 | PHE | 399 | 12.582 | 17.538 | 41.430 | 1.00 | 0.00 | 0.127 | 0.00 | 0.00 |
| ATOM | 4811 | CD2 | PHE | 399 | 15.441 | 19.181 | 40.673 | 1.00 | 0.00 | −0.127 | 10.80 | 0.60 |
| ATOM | 4812 | HD2 | PHE | 399 | 16.105 | 19.376 | 39.831 | 1.00 | 0.00 | 0.127 | 0.00 | 0.00 |
| ATOM | 4813 | CE1 | PHE | 399 | 13.754 | 18.690 | 42.833 | 1.00 | 0.00 | −0.127 | 10.80 | 0.60 |
| ATOM | 4814 | HE1 | PHE | 399 | 13.089 | 18.492 | 43.674 | 1.00 | 0.00 | 0.127 | 0.00 | 0.00 |
| ATOM | 4815 | CE2 | PHE | 399 | 15.735 | 19.720 | 41.917 | 1.00 | 0.00 | −0.127 | 10.80 | 0.60 |
| ATOM | 4816 | HE2 | PHE | 399 | 16.625 | 20.334 | 42.047 | 1.00 | 0.00 | 0.127 | 0.00 | 0.00 |
| ATOM | 4817 | CZ | PHE | 399 | 14.889 | 19.476 | 43.005 | 1.00 | 0.00 | −0.127 | 10.80 | 0.60 |
| ATOM | 4818 | HZ | PHE | 399 | 15.118 | 19.899 | 43.982 | 1.00 | 0.00 | 0.127 | 0.00 | 0.00 |
| ATOM | 4819 | C | PHE | 399 | 14.619 | 17.497 | 36.714 | 1.00 | 0.00 | 0.396 | 9.82 | 4.00 |
| ATOM | 4820 | O | PHE | 399 | 15.227 | 16.474 | 36.387 | 1.00 | 0.00 | −0.396 | 8.17 | −17.40 |
| ATOM | 4821 | N | ALA | 400 | 13.656 | 18.040 | 35.966 | 1.00 | 0.00 | −0.650 | 9.00 | −17.40 |
| ATOM | 4822 | HN | ALA | 400 | 13.181 | 18.888 | 36.304 | 1.00 | 0.00 | 0.440 | 0.00 | 0.00 |
| ATOM | 4823 | CA | ALA | 400 | 13.253 | 17.468 | 34.680 | 1.00 | 0.00 | 0.158 | 9.40 | 4.00 |
| ATOM | 4824 | HA | ALA | 400 | 12.856 | 16.464 | 34.833 | 1.00 | 0.00 | 0.053 | 0.00 | 0.00 |
| ATOM | 4825 | CB | ALA | 400 | 12.158 | 18.330 | 34.037 | 1.00 | 0.00 | −0.159 | 16.15 | 4.00 |
| ATOM | 4826 | HB1 | ALA | 400 | 11.866 | 17.894 | 33.081 | 1.00 | 0.00 | 0.053 | 0.00 | 0.00 |
| ATOM | 4827 | HB2 | ALA | 400 | 11.291 | 18.369 | 34.697 | 1.00 | 0.00 | 0.053 | 0.00 | 0.00 |
| ATOM | 4828 | HB3 | ALA | 400 | 12.537 | 19.339 | 33.875 | 1.00 | 0.00 | 0.053 | 0.00 | 0.00 |
| ATOM | 4829 | C | ALA | 400 | 14.461 | 17.396 | 33.748 | 1.00 | 0.00 | 0.396 | 9.82 | 4.00 |
| ATOM | 4830 | O | ALA | 400 | 14.760 | 16.355 | 33.164 | 1.00 | 0.00 | −0.396 | 8.17 | −17.40 |
| ATOM | 4831 | N | LYS | 401S | 15.141 | 18.529 | 33.616 | 1.00 | 0.00 | −0.650 | 9.00 | −17.40 |
| ATOM | 4832 | HN | LYS | 401S | 14.820 | 19.359 | 34.134 | 1.00 | 0.00 | 0.440 | 0.00 | 0.00 |
| ATOM | 4833 | CA | LYS | 401S | 16.318 | 18.647 | 32.772 | 1.00 | 0.00 | 0.158 | 9.40 | 4.00 |
| ATOM | 4834 | HA | LYS | 401S | 16.078 | 18.348 | 31.751 | 1.00 | 0.00 | 0.053 | 0.00 | 0.00 |
| ATOM | 4835 | CB | LYS | 401S | 16.755 | 20.121 | 32.746 | 1.00 | 0.00 | −0.106 | 12.77 | 4.00 |
| ATOM | 4836 | HB1 | LYS | 401S | 17.064 | 20.396 | 33.754 | 1.00 | 0.00 | 0.053 | 0.00 | 0.00 |
| ATOM | 4837 | HB2 | LYS | 401S | 15.902 | 20.717 | 32.422 | 1.00 | 0.00 | 0.053 | 0.00 | 0.00 |
| ATOM | 4838 | CG | LYS | 401S | 17.911 | 20.454 | 31.818 | 1.00 | 0.00 | −0.106 | 12.77 | 4.00 |
| ATOM | 4839 | HG1 | LYS | 401S | 17.794 | 19.909 | 30.880 | 1.00 | 0.00 | 0.053 | 0.00 | 0.00 |
| ATOM | 4840 | HG2 | LYS | 401S | 18.851 | 20.167 | 32.289 | 1.00 | 0.00 | 0.053 | 0.00 | 0.00 |
| ATOM | 4841 | CD | LYS | 401S | 17.923 | 21.958 | 31.534 | 1.00 | 0.00 | −0.106 | 12.77 | 4.00 |
| ATOM | 4842 | HD1 | LYS | 401S | 17.695 | 22.540 | 32.426 | 1.00 | 0.00 | 0.053 | 0.00 | 0.00 |
| ATOM | 4843 | HD2 | LYS | 401S | 17.188 | 22.233 | 30.777 | 1.00 | 0.00 | 0.053 | 0.00 | 0.00 |
| ATOM | 4844 | CE | LYS | 401S | 19.282 | 22.428 | 31.028 | 1.00 | 0.00 | 0.099 | 12.77 | 4.00 |
| ATOM | 4845 | HE1 | LYS | 401S | 19.259 | 23.480 | 30.744 | 1.00 | 0.00 | 0.053 | 0.00 | 0.00 |
| ATOM | 4846 | HE2 | LYS | 401S | 19.599 | 21.860 | 30.153 | 1.00 | 0.00 | 0.053 | 0.00 | 0.00 |
| ATOM | 4847 | NZ | LYS | 401S | 20.344 | 22.268 | 32.078 | 1.00 | 0.00 | −0.045 | 13.25 | −39.20 |
| ATOM | 4848 | HZ1 | LYS | 401S | 21.247 | 22.593 | 31.705 | 1.00 | 0.00 | 0.280 | 0.00 | 0.00 |
| ATOM | 4849 | HZ2 | LYS | 401S | 20.094 | 22.825 | 32.907 | 1.00 | 0.00 | 0.280 | 0.00 | 0.00 |
| ATOM | 4850 | HZ3 | LYS | 401S | 20.419 | 21.275 | 32.342 | 1.00 | 0.00 | 0.280 | 0.00 | 0.00 |
| ATOM | 4851 | C | LYS | 401S | 17.442 | 17.743 | 33.303 | 1.00 | 0.00 | 0.396 | 9.82 | 4.00 |
| ATOM | 4852 | O | LYS | 401S | 18.121 | 17.060 | 32.542 | 1.00 | 0.00 | −0.396 | 8.17 | −17.40 |
| ATOM | 4853 | N | ALA | 402 | 17.622 | 17.711 | 34.613 | 1.00 | 0.00 | −0.650 | 9.00 | −17.40 |
| ATOM | 4854 | HN | ALA | 402 | 17.021 | 18.277 | 35.228 | 1.00 | 0.00 | 0.440 | 0.00 | 0.00 |
| ATOM | 4855 | CA | ALA | 402 | 18.665 | 16.879 | 35.185 | 1.00 | 0.00 | 0.158 | 9.40 | 4.00 |
| ATOM | 4856 | HA | ALA | 402 | 19.660 | 17.134 | 34.822 | 1.00 | 0.00 | 0.053 | 0.00 | 0.00 |
| ATOM | 4857 | CB | ALA | 402 | 18.727 | 17.098 | 36.685 | 1.00 | 0.00 | −0.159 | 16.15 | 4.00 |
| ATOM | 4858 | HB1 | ALA | 402 | 19.510 | 16.472 | 37.112 | 1.00 | 0.00 | 0.053 | 0.00 | 0.00 |
| ATOM | 4859 | HB2 | ALA | 402 | 18.946 | 18.145 | 36.891 | 1.00 | 0.00 | 0.053 | 0.00 | 0.00 |
| ATOM | 4860 | HB3 | ALA | 402 | 17.768 | 16.833 | 37.131 | 1.00 | 0.00 | 0.053 | 0.00 | 0.00 |
| ATOM | 4861 | C | ALA | 402 | 18.480 | 15.384 | 34.874 | 1.00 | 0.00 | 0.396 | 9.82 | 4.00 |
| ATOM | 4862 | O | ALA | 402 | 19.461 | 14.647 | 34.746 | 1.00 | 0.00 | −0.396 | 8.17 | −17.40 |
| ATOM | 4863 | N | LEU | 403 | 17.239 | 14.933 | 34.732 | 1.00 | 0.00 | −0.650 | 9.00 | −17.40 |
| ATOM | 4864 | HN | LEU | 403 | 16.442 | 15.581 | 34.812 | 1.00 | 0.00 | 0.440 | 0.00 | 0.00 |
| ATOM | 4865 | CA | LEU | 403 | 17.003 | 13.514 | 34.461 | 1.00 | 0.00 | 0.158 | 9.40 | 4.00 |
| ATOM | 4866 | HA | LEU | 403 | 17.914 | 12.967 | 34.703 | 1.00 | 0.00 | 0.053 | 0.00 | 0.00 |
| ATOM | 4867 | CB | LEU | 403 | 15.935 | 12.977 | 35.418 | 1.00 | 0.00 | −0.106 | 12.77 | 4.00 |
| ATOM | 4868 | HB1 | LEU | 403 | 15.794 | 11.898 | 35.351 | 1.00 | 0.00 | 0.053 | 0.00 | 0.00 |
| ATOM | 4869 | HB2 | LEU | 403 | 14.946 | 13.404 | 35.250 | 1.00 | 0.00 | 0.053 | 0.00 | 0.00 |
| ATOM | 4870 | CG | LEU | 403 | 16.244 | 13.250 | 36.895 | 1.00 | 0.00 | −0.053 | 9.40 | 4.00 |
| ATOM | 4871 | HG | LEU | 403 | 16.466 | 14.307 | 37.040 | 1.00 | 0.00 | 0.053 | 0.00 | 0.00 |
| ATOM | 4872 | CD1 | LEU | 403 | 15.056 | 12.878 | 37.748 | 1.00 | 0.00 | −0.159 | 16.15 | 4.00 |
| ATOM | 4873 | HD1 | LEU | 403 | 15.284 | 13.075 | 38.795 | 1.00 | 0.00 | 0.053 | 0.00 | 0.00 |
| ATOM | 4874 | HD1 | LEU | 403 | 14.192 | 13.470 | 37.447 | 1.00 | 0.00 | 0.053 | 0.00 | 0.00 |
| ATOM | 4875 | HD1 | LEU | 403 | 14.832 | 11.819 | 37.618 | 1.00 | 0.00 | 0.053 | 0.00 | 0.00 |
| ATOM | 4876 | CD2 | LEU | 403 | 17.486 | 12.482 | 37.325 | 1.00 | 0.00 | −0.159 | 16.15 | 4.00 |
| ATOM | 4877 | HD2 | LEU | 403 | 17.693 | 12.685 | 38.375 | 1.00 | 0.00 | 0.053 | 0.00 | 0.00 |
| ATOM | 4878 | HD2 | LEU | 403 | 17.318 | 11.413 | 37.188 | 1.00 | 0.00 | 0.053 | 0.00 | 0.00 |
| ATOM | 4879 | HD2 | LEU | 403 | 18.336 | 12.796 | 36.719 | 1.00 | 0.00 | 0.053 | 0.00 | 0.00 |
| ATOM | 4880 | C | LEU | 403 | 16.640 | 13.178 | 33.008 | 1.00 | 0.00 | 0.396 | 9.82 | 4.00 |
| ATOM | 4881 | O | LEU | 403 | 16.375 | 12.018 | 32.687 | 1.00 | 0.00 | −0.396 | 8.17 | −17.40 |
| ATOM | 4882 | N | GLY | 404 | 16.632 | 14.187 | 32.138 | 1.00 | 0.00 | −0.650 | 9.00 | −17.40 |
| ATOM | 4883 | HN | GLY | 404 | 16.826 | 15.143 | 32.468 | 1.00 | 0.00 | 0.440 | 0.00 | 0.00 |
| ATOM | 4884 | CA | GLY | 404 | 16.353 | 13.958 | 30.729 | 1.00 | 0.00 | 0.105 | 9.40 | 4.00 |
| ATOM | 4885 | HA1 | GLY | 404 | 16.786 | 12.992 | 30.467 | 1.00 | 0.00 | 0.053 | 0.00 | 0.00 |
| ATOM | 4886 | HA2 | GLY | 404 | 16.817 | 14.771 | 30.172 | 1.00 | 0.00 | 0.053 | 0.00 | 0.00 |
| ATOM | 4887 | C | GLY | 404 | 14.906 | 13.907 | 30.269 | 1.00 | 0.00 | 0.396 | 9.82 | 4.00 |
| ATOM | 4888 | O | GLY | 404 | 14.614 | 13.380 | 29.189 | 1.00 | 0.00 | −0.396 | 8.17 | −17.40 |
| ATOM | 4889 | N | VAL | 405 | 14.004 | 14.458 | 31.079 | 1.00 | 0.00 | −0.650 | 9.00 | −17.40 |
| ATOM | 4890 | HN | VAL | 405 | 14.325 | 14.875 | 31.964 | 1.00 | 0.00 | 0.440 | 0.00 | 0.00 |
| ATOM | 4891 | CA | VAL | 405 | 12.576 | 14.495 | 30.765 | 1.00 | 0.00 | 0.158 | 9.40 | 4.00 |
| ATOM | 4892 | HA | VAL | 405 | 12.332 | 13.622 | 30.158 | 1.00 | 0.00 | 0.053 | 0.00 | 0.00 |
| ATOM | 4893 | CB | VAL | 405 | 11.723 | 14.509 | 32.064 | 1.00 | 0.00 | −0.053 | 9.40 | 4.00 |
| ATOM | 4894 | HB | VAL | 405 | 12.067 | 15.303 | 32.725 | 1.00 | 0.00 | 0.053 | 0.00 | 0.00 |
| ATOM | 4895 | CG1 | VAL | 405 | 10.258 | 14.749 | 31.720 | 1.00 | 0.00 | −0.159 | 16.15 | 4.00 |
| ATOM | 4896 | HG1 | VAL | 405 | 9.666 | 14.757 | 32.635 | 1.00 | 0.00 | 0.053 | 0.00 | 0.00 |
| ATOM | 4897 | HG1 | VAL | 405 | 10.155 | 15.708 | 31.212 | 1.00 | 0.00 | 0.053 | 0.00 | 0.00 |
| ATOM | 4898 | HG1 | VAL | 405 | 9.902 | 13.953 | 31.065 | 1.00 | 0.00 | 0.053 | 0.00 | 0.00 |
| ATOM | 4899 | CG2 | VAL | 405 | 11.890 | 13.204 | 32.817 | 1.00 | 0.00 | −0.159 | 16.15 | 4.00 |
| ATOM | 4900 | HG2 | VAL | 405 | 11.286 | 13.227 | 33.724 | 1.00 | 0.00 | 0.053 | 0.00 | 0.00 |
| ATOM | 4901 | HG2 | VAL | 405 | 11.564 | 12.376 | 32.187 | 1.00 | 0.00 | 0.053 | 0.00 | 0.00 |
| ATOM | 4902 | HG2 | VAL | 405 | 12.938 | 13.068 | 33.081 | 1.00 | 0.00 | 0.053 | 0.00 | 0.00 |
| ATOM | 4903 | C | VAL | 405 | 12.283 | 15.779 | 29.994 | 1.00 | 0.00 | 0.396 | 9.82 | 4.00 |
| ATOM | 4904 | O | VAL | 405 | 12.974 | 16.789 | 30.189 | 1.00 | 0.00 | −0.396 | 8.17 | −17.40 |
| ATOM | 4905 | N | MET | 406 | 11.282 | 15.752 | 29.115 | 1.00 | 0.00 | −0.650 | 9.00 | −17.40 |
| ATOM | 4906 | HN | MET | 406 | 10.753 | 14.881 | 28.961 | 1.00 | 0.00 | 0.440 | 0.00 | 0.00 |
| ATOM | 4907 | CA | MET | 406 | 10.935 | 16.960 | 28.370 | 1.00 | 0.00 | 0.158 | 9.40 | 4.00 |
| ATOM | 4908 | HA | MET | 406 | 11.792 | 17.337 | 27.813 | 1.00 | 0.00 | 0.053 | 0.00 | 0.00 |
| ATOM | 4909 | CB | MET | 406 | 9.789 | 16.709 | 27.373 | 1.00 | 0.00 | −0.106 | 12.77 | 4.00 |
| ATOM | 4910 | HB1 | MET | 406 | 8.943 | 17.334 | 27.660 | 1.00 | 0.00 | 0.053 | 0.00 | 0.00 |
| ATOM | 4911 | HB2 | MET | 406 | 9.520 | 15.653 | 27.420 | 1.00 | 0.00 | 0.053 | 0.00 | 0.00 |
| ATOM | 4912 | CG | MET | 406 | 10.137 | 17.038 | 25.904 | 1.00 | 0.00 | −0.041 | 12.77 | 4.00 |
| ATOM | 4913 | HG1 | MET | 406 | 9.202 | 17.183 | 25.362 | 1.00 | 0.00 | 0.053 | 0.00 | 0.00 |
| ATOM | 4914 | HG2 | MET | 406 | 10.698 | 16.197 | 25.496 | 1.00 | 0.00 | 0.053 | 0.00 | 0.00 |
| ATOM | 4915 | SD | MET | 406 | 11.159 | 18.553 | 25.639 | 1.00 | 0.00 | −0.130 | 16.39 | −6.40 |
| ATOM | 4916 | CE | MET | 406 | 9.938 | 19.798 | 25.460 | 1.00 | 0.00 | −0.094 | 16.15 | 4.00 |
| ATOM | 4917 | HE1 | MET | 406 | 10.421 | 20.760 | 25.294 | 1.00 | 0.00 | 0.053 | 0.00 | 0.00 |
| ATOM | 4918 | HE2 | MET | 406 | 9.299 | 19.561 | 24.608 | 1.00 | 0.00 | 0.053 | 0.00 | 0.00 |
| ATOM | 4919 | HE3 | MET | 406 | 9.332 | 19.846 | 26.365 | 1.00 | 0.00 | 0.053 | 0.00 | 0.00 |
| ATOM | 4920 | C | MET | 406 | 10.498 | 17.924 | 29.460 | 1.00 | 0.00 | 0.396 | 9.82 | 4.00 |
| ATOM | 4921 | O | MET | 406 | 9.661 | 17.582 | 30.292 | 1.00 | 0.00 | −0.396 | 8.17 | −17.40 |
| ATOM | 4922 | N | ASP | 407P | 11.075 | 19.123 | 29.461 | 1.00 | 0.00 | −0.650 | 9.00 | −17.40 |
| ATOM | 4923 | HN | ASP | 407P | 11.738 | 19.366 | 28.711 | 1.00 | 0.00 | 0.440 | 0.00 | 0.00 |
| ATOM | 4924 | CA | ASP | 407P | 10.786 | 20.094 | 30.500 | 1.00 | 0.00 | 0.158 | 9.40 | 4.00 |
| ATOM | 4925 | HA | ASP | 407P | 10.340 | 19.549 | 31.332 | 1.00 | 0.00 | 0.053 | 0.00 | 0.00 |
| ATOM | 4926 | CB | ASP | 407P | 12.109 | 20.657 | 31.034 | 1.00 | 0.00 | −0.336 | 12.77 | 4.00 |
| ATOM | 4927 | HB1 | ASP | 407P | 12.762 | 19.887 | 31.445 | 1.00 | 0.00 | 0.053 | 0.00 | 0.00 |
| ATOM | 4928 | HB2 | ASP | 407P | 11.969 | 21.385 | 31.832 | 1.00 | 0.00 | 0.053 | 0.00 | 0.00 |
| ATOM | 4929 | CG | ASP | 407P | 12.924 | 21.365 | 29.967 | 1.00 | 0.00 | 0.297 | 9.82 | 4.00 |
| ATOM | 4930 | OD1 | ASP | 407P | 12.695 | 21.142 | 28.764 | 1.00 | 0.00 | −0.534 | 8.17 | −18.95 |
| ATOM | 4931 | OD2 | ASP | 407P | 13.827 | 22.139 | 30.341 | 1.00 | 0.00 | −0.534 | 8.17 | −18.95 |
| ATOM | 4932 | C | ASP | 407P | 9.834 | 21.246 | 30.197 | 1.00 | 0.00 | 0.396 | 9.82 | 4.00 |
| ATOM | 4933 | O | ASP | 407P | 9.622 | 22.097 | 31.064 | 1.00 | 0.00 | −0.396 | 8.17 | −17.40 |
| ATOM | 4934 | N | ASP | 408P | 9.247 | 21.291 | 29.004 | 1.00 | 0.00 | −0.650 | 9.00 | −17.40 |
| ATOM | 4935 | HN | ASP | 408P | 9.434 | 20.560 | 28.302 | 1.00 | 0.00 | 0.440 | 0.00 | 0.00 |
| ATOM | 4936 | CA | ASP | 408P | 8.332 | 22.392 | 28.703 | 1.00 | 0.00 | 0.158 | 9.40 | 4.00 |
| ATOM | 4937 | HA | ASP | 408P | 8.741 | 23.309 | 29.126 | 1.00 | 0.00 | 0.053 | 0.00 | 0.00 |
| ATOM | 4938 | CB | ASP | 408P | 8.215 | 22.605 | 27.179 | 1.00 | 0.00 | −0.336 | 12.77 | 4.00 |
| ATOM | 4939 | HB1 | ASP | 408P | 9.179 | 22.695 | 26.679 | 1.00 | 0.00 | 0.053 | 0.00 | 0.00 |
| ATOM | 4940 | HB2 | ASP | 408P | 7.665 | 23.506 | 26.908 | 1.00 | 0.00 | 0.053 | 0.00 | 0.00 |
| ATOM | 4941 | CG | ASP | 408P | 7.494 | 21.463 | 26.459 | 1.00 | 0.00 | 0.297 | 9.82 | 4.00 |
| ATOM | 4942 | OD1 | ASP | 408P | 7.611 | 20.296 | 26.894 | 1.00 | 0.00 | −0.534 | 8.17 | −18.95 |
| ATOM | 4943 | OD2 | ASP | 408P | 6.827 | 21.733 | 25.432 | 1.00 | 0.00 | −0.534 | 8.17 | −18.95 |
| ATOM | 4944 | C | ASP | 408P | 6.979 | 22.081 | 29.323 | 1.00 | 0.00 | 0.396 | 9.82 | 4.00 |
| ATOM | 4945 | O | ASP | 408P | 6.744 | 20.944 | 29.753 | 1.00 | 0.00 | −0.396 | 8.17 | −17.40 |
| ATOM | 4946 | N | LEU | 409 | 6.113 | 23.088 | 29.422 | 1.00 | 0.00 | −0.650 | 9.00 | −17.40 |
| ATOM | 4947 | HN | LEU | 409 | 6.387 | 24.023 | 29.089 | 1.00 | 0.00 | 0.440 | 0.00 | 0.00 |
| ATOM | 4948 | CA | LEU | 409 | 4.783 | 22.894 | 29.993 | 1.00 | 0.00 | 0.158 | 9.40 | 4.00 |
| ATOM | 4949 | HA | LEU | 409 | 4.532 | 21.839 | 30.113 | 1.00 | 0.00 | 0.053 | 0.00 | 0.00 |
| ATOM | 4950 | CB | LEU | 409 | 4.713 | 23.527 | 31.385 | 1.00 | 0.00 | −0.106 | 12.77 | 4.00 |
| ATOM | 4951 | HB1 | LEU | 409 | 3.690 | 23.424 | 31.749 | 1.00 | 0.00 | 0.053 | 0.00 | 0.00 |
| ATOM | 4952 | HB2 | LEU | 409 | 4.989 | 24.577 | 31.291 | 1.00 | 0.00 | 0.053 | 0.00 | 0.00 |
| ATOM | 4953 | CG | LEU | 409 | 5.633 | 22.910 | 32.440 | 1.00 | 0.00 | −0.053 | 9.40 | 4.00 |
| ATOM | 4954 | HG | LEU | 409 | 6.098 | 22.008 | 32.041 | 1.00 | 0.00 | 0.053 | 0.00 | 0.00 |
| ATOM | 4955 | CD1 | LEU | 409 | 6.718 | 23.905 | 32.822 | 1.00 | 0.00 | −0.159 | 16.15 | 4.00 |
| ATOM | 4956 | HD1 | LEU | 409 | 7.370 | 23.460 | 33.573 | 1.00 | 0.00 | 0.053 | 0.00 | 0.00 |
| ATOM | 4957 | HD1 | LEU | 409 | 7.303 | 24.161 | 31.939 | 1.00 | 0.00 | 0.053 | 0.00 | 0.00 |
| ATOM | 4958 | HD1 | LEU | 409 | 6.258 | 24.806 | 33.227 | 1.00 | 0.00 | 0.053 | 0.00 | 0.00 |
| ATOM | 4959 | CD2 | LEU | 409 | 4.812 | 22.504 | 33.650 | 1.00 | 0.00 | −0.159 | 16.15 | 4.00 |
| ATOM | 4960 | HD2 | LEU | 409 | 5.466 | 22.064 | 34.402 | 1.00 | 0.00 | 0.053 | 0.00 | 0.00 |
| ATOM | 4961 | HD2 | LEU | 409 | 4.318 | 23.382 | 34.066 | 1.00 | 0.00 | 0.053 | 0.00 | 0.00 |
| ATOM | 4962 | HD2 | LEU | 409 | 4.060 | 21.773 | 33.351 | 1.00 | 0.00 | 0.053 | 0.00 | 0.00 |
| ATOM | 4963 | C | LEU | 409 | 3.678 | 23.506 | 29.121 | 1.00 | 0.00 | 0.396 | 9.82 | 4.00 |
| ATOM | 4964 | O | LEU | 409 | 3.859 | 24.596 | 28.566 | 1.00 | 0.00 | −0.396 | 8.17 | −17.40 |
| ATOM | 4965 | N | LYS | 410S | 2.534 | 22.827 | 28.991 | 1.00 | 0.00 | −0.650 | 9.00 | −17.40 |
| ATOM | 4966 | HN | LYS | 410S | 2.428 | 21.905 | 29.438 | 1.00 | 0.00 | 0.440 | 0.00 | 0.00 |
| ATOM | 4967 | CA | LYS | 410S | 1.428 | 23.395 | 28.211 | 1.00 | 0.00 | 0.158 | 9.40 | 4.00 |
| ATOM | 4968 | HA | LYS | 410S | 1.779 | 24.325 | 27.765 | 1.00 | 0.00 | 0.053 | 0.00 | 0.00 |
| ATOM | 4969 | CB | LYS | 410S | 1.010 | 22.459 | 27.068 | 1.00 | 0.00 | −0.106 | 12.77 | 4.00 |
| ATOM | 4970 | HB1 | LYS | 410S | 0.173 | 22.916 | 26.540 | 1.00 | 0.00 | 0.053 | 0.00 | 0.00 |
| ATOM | 4971 | HB2 | LYS | 410S | 0.715 | 21.502 | 27.500 | 1.00 | 0.00 | 0.053 | 0.00 | 0.00 |
| ATOM | 4972 | CG | LYS | 410S | 2.116 | 22.183 | 26.036 | 1.00 | 0.00 | −0.106 | 12.77 | 4.00 |
| ATOM | 4973 | HG1 | LYS | 410S | 1.820 | 21.423 | 25.312 | 1.00 | 0.00 | 0.053 | 0.00 | 0.00 |
| ATOM | 4974 | HG2 | LYS | 410S | 3.034 | 21.829 | 26.504 | 1.00 | 0.00 | 0.053 | 0.00 | 0.00 |
| ATOM | 4975 | CD | LYS | 410S | 2.480 | 23.428 | 25.242 | 1.00 | 0.00 | −0.106 | 12.77 | 4.00 |
| ATOM | 4976 | HD1 | LYS | 410S | 3.026 | 24.146 | 25.852 | 1.00 | 0.00 | 0.053 | 0.00 | 0.00 |
| ATOM | 4977 | HD2 | LYS | 410S | 1.592 | 23.934 | 24.862 | 1.00 | 0.00 | 0.053 | 0.00 | 0.00 |
| ATOM | 4978 | CE | LYS | 410S | 3.363 | 23.089 | 24.036 | 1.00 | 0.00 | 0.099 | 12.77 | 4.00 |
| ATOM | 4979 | HE1 | LYS | 410S | 2.905 | 22.330 | 23.401 | 1.00 | 0.00 | 0.053 | 0.00 | 0.00 |
| ATOM | 4980 | HE2 | LYS | 410S | 4.336 | 22.705 | 24.343 | 1.00 | 0.00 | 0.053 | 0.00 | 0.00 |
| ATOM | 4981 | NZ | LYS | 410S | 3.615 | 24.299 | 23.171 | 1.00 | 0.00 | −0.045 | 13.25 | −39.20 |
| ATOM | 4982 | HZ1 | LYS | 410S | 4.209 | 24.033 | 22.373 | 1.00 | 0.00 | 0.280 | 0.00 | 0.00 |
| ATOM | 4983 | HZ2 | LYS | 410S | 2.717 | 24.665 | 22.822 | 1.00 | 0.00 | 0.280 | 0.00 | 0.00 |
| ATOM | 4984 | HZ3 | LYS | 410S | 4.089 | 25.025 | 23.726 | 1.00 | 0.00 | 0.280 | 0.00 | 0.00 |
| ATOM | 4985 | C | LYS | 410S | 0.266 | 23.647 | 29.168 | 1.00 | 0.00 | 0.396 | 9.82 | 4.00 |
| ATOM | 4986 | O | LYS | 410S | −0.254 | 22.722 | 29.794 | 1.00 | 0.00 | −0.396 | 8.17 | −17.40 |
| ATOM | 4987 | N | SER | 411 | −0.153 | 24.904 | 29.276 | 1.00 | 0.00 | −0.650 | 9.00 | −17.40 |
| ATOM | 4988 | HN | SER | 411 | 0.275 | 25.630 | 28.685 | 1.00 | 0.00 | 0.440 | 0.00 | 0.00 |
| ATOM | 4989 | CA | SER | 411 | −1.208 | 25.276 | 30.215 | 1.00 | 0.00 | 0.158 | 9.40 | 4.00 |
| ATOM | 4990 | HA | SER | 411 | −1.316 | 26.360 | 30.247 | 1.00 | 0.00 | 0.053 | 0.00 | 0.00 |
| ATOM | 4991 | CB | SER | 411 | −2.575 | 24.744 | 29.766 | 1.00 | 0.00 | 0.007 | 12.77 | 4.00 |
| ATOM | 4992 | HB1 | SER | 411 | −3.293 | 24.834 | 30.581 | 1.00 | 0.00 | 0.053 | 0.00 | 0.00 |
| ATOM | 4993 | HB2 | SER | 411 | −2.488 | 23.695 | 29.481 | 1.00 | 0.00 | 0.053 | 0.00 | 0.00 |
| ATOM | 4994 | OG | SER | 411 | −3.042 | 25.493 | 28.648 | 1.00 | 0.00 | −0.537 | 11.04 | −17.40 |
| ATOM | 4995 | HG | SER | 411 | −2.644 | 26.442 | 28.680 | 1.00 | 0.00 | 0.424 | 0.00 | 0.00 |
| ATOM | 4996 | C | SER | 411 | −0.865 | 24.773 | 31.618 | 1.00 | 0.00 | 0.396 | 9.82 | 4.00 |
| ATOM | 4997 | O | SER | 411 | −1.746 | 24.405 | 32.403 | 1.00 | 0.00 | −0.396 | 8.17 | −17.40 |
| ATOM | 4998 | N | GLY | 412 | 0.431 | 24.757 | 31.928 | 1.00 | 0.00 | −0.650 | 9.00 | −17.40 |
| ATOM | 4999 | HN | GLY | 412 | 1.128 | 25.045 | 31.227 | 1.00 | 0.00 | 0.440 | 0.00 | 0.00 |
| ATOM | 5000 | CA | GLY | 412 | 0.868 | 24.334 | 33.253 | 1.00 | 0.00 | 0.105 | 9.40 | 4.00 |
| ATOM | 5001 | HA1 | GLY | 412 | 0.102 | 24.635 | 33.968 | 1.00 | 0.00 | 0.053 | 0.00 | 0.00 |
| ATOM | 5002 | HA2 | GLY | 412 | 1.817 | 24.826 | 33.463 | 1.00 | 0.00 | 0.053 | 0.00 | 0.00 |
| ATOM | 5003 | C | GLY | 412 | 1.091 | 22.835 | 33.445 | 1.00 | 0.00 | 0.396 | 9.82 | 4.00 |
| ATOM | 5004 | O | GLY | 412 | 1.614 | 22.421 | 34.468 | 1.00 | 0.00 | −0.396 | 8.17 | −17.40 |
| ATOM | 5005 | N | VAL | 413 | 0.706 | 22.027 | 32.460 | 1.00 | 0.00 | −0.650 | 9.00 | −17.40 |
| ATOM | 5006 | HN | VAL | 413 | 0.296 | 22.439 | 31.610 | 1.00 | 0.00 | 0.440 | 0.00 | 0.00 |
| ATOM | 5007 | CA | VAL | 413 | 0.845 | 20.565 | 32.543 | 1.00 | 0.00 | 0.158 | 9.40 | 4.00 |
| ATOM | 5008 | HA | VAL | 413 | 0.832 | 20.183 | 33.564 | 1.00 | 0.00 | 0.053 | 0.00 | 0.00 |
| ATOM | 5009 | CB | VAL | 413 | −0.312 | 19.865 | 31.794 | 1.00 | 0.00 | −0.053 | 9.40 | 4.00 |
| ATOM | 5010 | HB | VAL | 413 | −0.274 | 20.090 | 30.728 | 1.00 | 0.00 | 0.053 | 0.00 | 0.00 |
| ATOM | 5011 | CG1 | VAL | 413 | −0.225 | 18.316 | 31.967 | 1.00 | 0.00 | −0.159 | 16.15 | 4.00 |
| ATOM | 5012 | HG1 | VAL | 413 | −1.048 | 17.843 | 31.432 | 1.00 | 0.00 | 0.053 | 0.00 | 0.00 |
| ATOM | 5013 | HG1 | VAL | 413 | 0.722 | 17.957 | 31.564 | 1.00 | 0.00 | 0.053 | 0.00 | 0.00 |
| ATOM | 5014 | HG1 | VAL | 413 | −0.287 | 18.064 | 33.025 | 1.00 | 0.00 | 0.053 | 0.00 | 0.00 |
| ATOM | 5015 | CG2 | VAL | 413 | −1.647 | 20.411 | 32.298 | 1.00 | 0.00 | −0.159 | 16.15 | 4.00 |
| ATOM | 5016 | HG2 | VAL | 413 | −2.463 | 19.918 | 31.770 | 1.00 | 0.00 | 0.053 | 0.00 | 0.00 |
| ATOM | 5017 | HG2 | VAL | 413 | −1.737 | 20.220 | 33.367 | 1.00 | 0.00 | 0.053 | 0.00 | 0.00 |
| ATOM | 5018 | HG2 | VAL | 413 | −1.693 | 21.484 | 32.116 | 1.00 | 0.00 | 0.053 | 0.00 | 0.00 |
| ATOM | 5019 | C | VAL | 413 | 2.141 | 20.042 | 31.940 | 1.00 | 0.00 | 0.396 | 9.82 | 4.00 |
| ATOM | 5020 | O | VAL | 413 | 2.458 | 20.348 | 30.792 | 1.00 | 0.00 | −0.396 | 8.17 | −17.40 |
| ATOM | 5021 | N | PRO | 414 | 2.911 | 19.246 | 32.701 | 1.00 | 0.00 | −0.422 | 9.00 | −17.40 |
| ATOM | 5022 | CD | PRO | 414 | 2.848 | 19.043 | 34.162 | 1.00 | 0.00 | 0.105 | 12.77 | 4.00 |
| ATOM | 5023 | HD1 | PRO | 414 | 1.844 | 18.758 | 34.479 | 1.00 | 0.00 | 0.053 | 0.00 | 0.00 |
| ATOM | 5024 | HD2 | PRO | 414 | 3.118 | 19.951 | 34.700 | 1.00 | 0.00 | 0.053 | 0.00 | 0.00 |
| ATOM | 5025 | CA | PRO | 414 | 4.164 | 18.709 | 32.153 | 1.00 | 0.00 | 0.158 | 9.40 | 4.00 |
| ATOM | 5026 | HA | PRO | 414 | 4.734 | 19.453 | 31.597 | 1.00 | 0.00 | 0.053 | 0.00 | 0.00 |
| ATOM | 5027 | CB | PRO | 414 | 4.937 | 18.288 | 33.402 | 1.00 | 0.00 | −0.106 | 12.77 | 4.00 |
| ATOM | 5028 | HB1 | PRO | 414 | 5.563 | 19.100 | 33.769 | 1.00 | 0.00 | 0.053 | 0.00 | 0.00 |
| ATOM | 5029 | HB2 | PRO | 414 | 5.584 | 17.436 | 33.194 | 1.00 | 0.00 | 0.053 | 0.00 | 0.00 |
| ATOM | 5030 | CG | PRO | 414 | 3.845 | 17.939 | 34.373 | 1.00 | 0.00 | −0.106 | 12.77 | 4.00 |
| ATOM | 5031 | HG1 | PRO | 414 | 4.220 | 17.917 | 35.396 | 1.00 | 0.00 | 0.053 | 0.00 | 0.00 |
| ATOM | 5032 | HG2 | PRO | 414 | 3.421 | 16.958 | 34.154 | 1.00 | 0.00 | 0.053 | 0.00 | 0.00 |
| ATOM | 5033 | C | PRO | 414 | 3.936 | 17.545 | 31.187 | 1.00 | 0.00 | 0.396 | 9.82 | 4.00 |
| ATOM | 5034 | O | PRO | 414 | 2.876 | 16.910 | 31.184 | 1.00 | 0.00 | −0.396 | 8.17 | −17.40 |
| ATOM | 5035 | N | ARG | 415G | 4.941 | 17.277 | 30.367 | 1.00 | 0.00 | −0.650 | 9.00 | −17.40 |
| ATOM | 5036 | HN | ARG | 415G | 5.792 | 17.853 | 30.431 | 1.00 | 0.00 | 0.440 | 0.00 | 0.00 |
| ATOM | 5037 | CA | ARG | 415G | 4.903 | 16.210 | 29.381 | 1.00 | 0.00 | 0.158 | 9.40 | 4.00 |
| ATOM | 5038 | HA | ARG | 415G | 4.109 | 16.396 | 28.657 | 1.00 | 0.00 | 0.053 | 0.00 | 0.00 |
| ATOM | 5039 | CB | ARG | 415G | 6.252 | 16.161 | 28.659 | 1.00 | 0.00 | −0.106 | 12.77 | 4.00 |
| ATOM | 5040 | HB1 | ARG | 415G | 6.262 | 15.284 | 28.010 | 1.00 | 0.00 | 0.053 | 0.00 | 0.00 |
| ATOM | 5041 | HB2 | ARG | 415G | 7.039 | 16.091 | 29.409 | 1.00 | 0.00 | 0.053 | 0.00 | 0.00 |
| ATOM | 5042 | CG | ARG | 415G | 6.539 | 17.371 | 27.801 | 1.00 | 0.00 | −0.106 | 12.77 | 4.00 |
| ATOM | 5043 | HG1 | ARG | 415G | 7.611 | 17.563 | 27.762 | 1.00 | 0.00 | 0.053 | 0.00 | 0.00 |
| ATOM | 5044 | HG2 | ARG | 415G | 6.042 | 18.251 | 28.208 | 1.00 | 0.00 | 0.053 | 0.00 | 0.00 |
| ATOM | 5045 | CD | ARG | 415G | 6.037 | 17.146 | 26.382 | 1.00 | 0.00 | 0.374 | 12.77 | 4.00 |
| ATOM | 5046 | HD1 | ARG | 415G | 4.999 | 16.812 | 26.365 | 1.00 | 0.00 | 0.053 | 0.00 | 0.00 |
| ATOM | 5047 | HD2 | ARG | 415G | 6.621 | 16.389 | 25.858 | 1.00 | 0.00 | 0.053 | 0.00 | 0.00 |
| ATOM | 5048 | NE | ARG | 415G | 6.104 | 18.361 | 25.583 | 1.00 | 0.00 | −0.819 | 9.00 | −24.67 |
| ATOM | 5049 | HE | ARG | 415G | 6.546 | 19.191 | 26.001 | 1.00 | 0.00 | 0.407 | 0.00 | 0.00 |
| ATOM | 5050 | CZ | ARG | 415G | 5.628 | 18.463 | 24.345 | 1.00 | 0.00 | 0.796 | 6.95 | 4.00 |
| ATOM | 5051 | NH1 | ARG | 415G | 5.051 | 17.409 | 23.767 | 1.00 | 0.00 | −0.746 | 9.00 | −24.67 |
| ATOM | 5052 | HH1 | ARG | 415G | 4.681 | 17.485 | 22.808 | 1.00 | 0.00 | 0.407 | 0.00 | 0.00 |
| ATOM | 5053 | HH1 | ARG | 415G | 4.974 | 16.518 | 24.279 | 1.00 | 0.00 | 0.407 | 0.00 | 0.00 |
| ATOM | 5054 | NH2 | ARG | 415G | 5.721 | 19.615 | 23.692 | 1.00 | 0.00 | −0.746 | 9.00 | −24.67 |
| ATOM | 5055 | HH2 | ARG | 415G | 5.352 | 19.694 | 22.733 | 1.00 | 0.00 | 0.407 | 0.00 | 0.00 |
| ATOM | 5056 | HH2 | ARG | 415G | 6.161 | 20.429 | 24.144 | 1.00 | 0.00 | 0.407 | 0.00 | 0.00 |
| ATOM | 5057 | C | ARG | 415G | 4.641 | 14.874 | 30.065 | 1.00 | 0.00 | 0.396 | 9.82 | 4.00 |
| ATOM | 5058 | O | ARG | 415G | 5.398 | 14.487 | 30.954 | 1.00 | 0.00 | −0.396 | 8.17 | −17.40 |
| ATOM | 5059 | N | ALA | 416 | 3.577 | 14.187 | 29.651 | 1.00 | 0.00 | −0.650 | 9.00 | −17.40 |
| ATOM | 5060 | HN | ALA | 416 | 3.004 | 14.589 | 28.895 | 1.00 | 0.00 | 0.440 | 0.00 | 0.00 |
| ATOM | 5061 | CA | ALA | 416 | 3.171 | 12.886 | 30.210 | 1.00 | 0.00 | 0.158 | 9.40 | 4.00 |
| ATOM | 5062 | HA | ALA | 416 | 2.309 | 12.445 | 29.707 | 1.00 | 0.00 | 0.053 | 0.00 | 0.00 |
| ATOM | 5063 | CB | ALA | 416 | 4.295 | 11.838 | 30.014 | 1.00 | 0.00 | −0.159 | 16.15 | 4.00 |
| ATOM | 5064 | HB1 | ALA | 416 | 3.978 | 10.882 | 30.432 | 1.00 | 0.00 | 0.053 | 0.00 | 0.00 |
| ATOM | 5065 | HB2 | ALA | 416 | 4.499 | 11.718 | 28.950 | 1.00 | 0.00 | 0.053 | 0.00 | 0.00 |
| ATOM | 5066 | HB3 | ALA | 416 | 5.199 | 12.174 | 30.521 | 1.00 | 0.00 | 0.053 | 0.00 | 0.00 |
| ATOM | 5067 | C | ALA | 416 | 2.779 | 12.951 | 31.691 | 1.00 | 0.00 | 0.396 | 9.82 | 4.00 |
| ATOM | 5068 | O | ALA | 416 | 2.675 | 11.919 | 32.375 | 1.00 | 0.00 | −0.396 | 8.17 | −17.40 |
| ATOM | 5069 | N | GLY | 417 | 2.554 | 14.157 | 32.192 | 1.00 | 0.00 | −0.650 | 9.00 | −17.40 |
| ATOM | 5070 | HN | GLY | 417 | 2.639 | 14.991 | 31.594 | 1.00 | 0.00 | 0.440 | 0.00 | 0.00 |
| ATOM | 5071 | CA | GLY | 417 | 2.185 | 14.293 | 33.593 | 1.00 | 0.00 | 0.105 | 9.40 | 4.00 |
| ATOM | 5072 | HA1 | GLY | 417 | 2.314 | 15.337 | 33.878 | 1.00 | 0.00 | 0.053 | 0.00 | 0.00 |
| ATOM | 5073 | HA2 | GLY | 417 | 2.839 | 13.648 | 34.180 | 1.00 | 0.00 | 0.053 | 0.00 | 0.00 |
| ATOM | 5074 | C | GLY | 417 | 0.756 | 13.907 | 33.918 | 1.00 | 0.00 | 0.396 | 9.82 | 4.00 |
| ATOM | 5075 | O | GLY | 417 | −0.144 | 14.019 | 33.082 | 1.00 | 0.00 | −0.396 | 8.17 | −17.40 |
| ATOM | 5076 | N | TYR | 418 | 0.560 | 13.433 | 35.142 | 1.00 | 0.00 | −0.650 | 9.00 | −17.40 |
| ATOM | 5077 | HN | TYR | 418 | 1.374 | 13.328 | 35.763 | 1.00 | 0.00 | 0.440 | 0.00 | 0.00 |
| ATOM | 5078 | CA | TYR | 418 | −0.754 | 13.053 | 35.650 | 1.00 | 0.00 | 0.158 | 9.40 | 4.00 |
| ATOM | 5079 | HA | TYR | 418 | −1.453 | 13.586 | 35.006 | 1.00 | 0.00 | 0.053 | 0.00 | 0.00 |
| ATOM | 5080 | CB | TYR | 418 | −0.960 | 11.539 | 35.611 | 1.00 | 0.00 | −0.106 | 12.77 | 4.00 |
| ATOM | 5081 | HB1 | TYR | 418 | −0.313 | 11.038 | 36.332 | 1.00 | 0.00 | 0.053 | 0.00 | 0.00 |
| ATOM | 5082 | HB2 | TYR | 418 | −0.732 | 11.139 | 34.622 | 1.00 | 0.00 | 0.053 | 0.00 | 0.00 |
| ATOM | 5083 | CG | TYR | 418 | −2.378 | 11.182 | 35.937 | 1.00 | 0.00 | 0.000 | 7.26 | 0.60 |
| ATOM | 5084 | CD1 | TYR | 418 | −3.413 | 11.539 | 35.076 | 1.00 | 0.00 | −0.127 | 10.80 | 0.60 |
| ATOM | 5085 | HD1 | TYR | 418 | −3.182 | 12.053 | 34.143 | 1.00 | 0.00 | 0.127 | 0.00 | 0.00 |
| ATOM | 5086 | CE1 | TYR | 418 | −4.738 | 11.249 | 35.389 | 1.00 | 0.00 | −0.127 | 10.80 | 0.60 |
| ATOM | 5087 | HE1 | TYR | 418 | −5.538 | 11.529 | 34.704 | 1.00 | 0.00 | 0.127 | 0.00 | 0.00 |
| ATOM | 5088 | CD2 | TYR | 418 | −2.701 | 10.531 | 37.127 | 1.00 | 0.00 | −0.127 | 10.80 | 0.60 |
| ATOM | 5089 | HD2 | TYR | 418 | −1.904 | 10.243 | 37.813 | 1.00 | 0.00 | 0.127 | 0.00 | 0.00 |
| ATOM | 5090 | CE2 | TYR | 418 | −4.023 | 10.242 | 37.455 | 1.00 | 0.00 | −0.127 | 10.80 | 0.60 |
| ATOM | 5091 | HE2 | TYR | 418 | −4.261 | 9.739 | 38.392 | 1.00 | 0.00 | 0.127 | 0.00 | 0.00 |
| ATOM | 5092 | CZ | TYR | 418 | −5.030 | 10.601 | 36.577 | 1.00 | 0.00 | 0.026 | 7.26 | 0.60 |
| ATOM | 5093 | OH | TYR | 418 | −6.327 | 10.294 | 36.871 | 1.00 | 0.00 | −0.451 | 10.94 | −17.40 |
| ATOM | 5094 | HH | TYR | 418 | −6.872 | 11.162 | 36.969 | 1.00 | 0.00 | 0.424 | 0.00 | 0.00 |
| ATOM | 5095 | C | TYR | 418 | −0.747 | 13.534 | 37.105 | 1.00 | 0.00 | 0.396 | 9.82 | 4.00 |
| ATOM | 5096 | O | TYR | 418 | 0.068 | 13.076 | 37.893 | 1.00 | 0.00 | −0.396 | 8.17 | −17.40 |
| ATOM | 5097 | N | ARG | 419G | −1.663 | 14.446 | 37.449 | 1.00 | 0.00 | −0.650 | 9.00 | −17.40 |
| ATOM | 5098 | HN | ARG | 419G | −2.360 | 14.731 | 36.747 | 1.00 | 0.00 | 0.440 | 0.00 | 0.00 |
| ATOM | 5099 | CA | ARG | 419G | −1.719 | 15.058 | 38.780 | 1.00 | 0.00 | 0.158 | 9.40 | 4.00 |
| ATOM | 5100 | HA | ARG | 419G | −2.459 | 15.856 | 38.726 | 1.00 | 0.00 | 0.053 | 0.00 | 0.00 |
| ATOM | 5101 | CB | ARG | 419G | −2.165 | 14.066 | 39.854 | 1.00 | 0.00 | −0.106 | 12.77 | 4.00 |
| ATOM | 5102 | HB1 | ARG | 419G | −2.204 | 14.502 | 40.852 | 1.00 | 0.00 | 0.053 | 0.00 | 0.00 |
| ATOM | 5103 | HB2 | ARG | 419G | −1.506 | 13.201 | 39.939 | 1.00 | 0.00 | 0.053 | 0.00 | 0.00 |
| ATOM | 5104 | CG | ARG | 419G | −3.568 | 13.491 | 39.602 | 1.00 | 0.00 | −0.106 | 12.77 | 4.00 |
| ATOM | 5105 | HG1 | ARG | 419G | −3.567 | 12.766 | 38.787 | 1.00 | 0.00 | 0.053 | 0.00 | 0.00 |
| ATOM | 5106 | HG2 | ARG | 419G | −4.282 | 14.270 | 39.337 | 1.00 | 0.00 | 0.053 | 0.00 | 0.00 |
| ATOM | 5107 | CD | ARG | 419G | −4.116 | 12.781 | 40.832 | 1.00 | 0.00 | 0.374 | 12.77 | 4.00 |
| ATOM | 5108 | HD1 | ARG | 419G | −3.445 | 11.990 | 41.168 | 1.00 | 0.00 | 0.053 | 0.00 | 0.00 |
| ATOM | 5109 | HD2 | ARG | 419G | −5.084 | 12.323 | 40.630 | 1.00 | 0.00 | 0.053 | 0.00 | 0.00 |
| ATOM | 5110 | NE | ARG | 419G | −4.295 | 13.708 | 41.950 | 1.00 | 0.00 | −0.819 | 9.00 | −24.67 |
| ATOM | 5111 | HE | ARG | 419G | −3.565 | 13.732 | 42.676 | 1.00 | 0.00 | 0.407 | 0.00 | 0.00 |
| ATOM | 5112 | CZ | ARG | 419G | −5.341 | 14.524 | 42.091 | 1.00 | 0.00 | 0.796 | 6.95 | 4.00 |
| ATOM | 5113 | NH1 | ARG | 419G | −6.315 | 14.527 | 41.187 | 1.00 | 0.00 | −0.746 | 9.00 | −24.67 |
| ATOM | 5114 | HH1 | ARG | 419G | −7.120 | 15.158 | 41.300 | 1.00 | 0.00 | 0.407 | 0.00 | 0.00 |
| ATOM | 5115 | HH1 | ARG | 419G | −6.264 | 13.897 | 40.373 | 1.00 | 0.00 | 0.407 | 0.00 | 0.00 |
| ATOM | 5116 | NH2 | ARG | 419G | −5.408 | 15.351 | 43.133 | 1.00 | 0.00 | −0.746 | 9.00 | −24.67 |
| ATOM | 5117 | HH2 | ARG | 419G | −6.216 | 15.980 | 43.240 | 1.00 | 0.00 | 0.407 | 0.00 | 0.00 |
| ATOM | 5118 | HH2 | ARG | 419G | −4.651 | 15.361 | 43.832 | 1.00 | 0.00 | 0.407 | 0.00 | 0.00 |
| ATOM | 5119 | C | ARG | 419G | −0.321 | 15.591 | 39.076 | 1.00 | 0.00 | 0.396 | 9.82 | 4.00 |
| ATOM | 5120 | O | ARG | 419G | 0.163 | 15.547 | 40.218 | 1.00 | 0.00 | −0.396 | 8.17 | −17.40 |
| ATOM | 5121 | N | GLY | 420 | 0.321 | 16.074 | 38.014 | 1.00 | 0.00 | −0.650 | 9.00 | −17.40 |
| ATOM | 5122 | HN | GLY | 420 | −0.147 | 16.029 | 37.097 | 1.00 | 0.00 | 0.440 | 0.00 | 0.00 |
| ATOM | 5123 | CA | GLY | 420 | 1.657 | 16.665 | 38.083 | 1.00 | 0.00 | 0.105 | 9.40 | 4.00 |
| ATOM | 5124 | HA1 | GLY | 420 | 1.723 | 17.239 | 39.007 | 1.00 | 0.00 | 0.053 | 0.00 | 0.00 |
| ATOM | 5125 | HA2 | GLY | 420 | 1.784 | 17.309 | 37.213 | 1.00 | 0.00 | 0.053 | 0.00 | 0.00 |
| ATOM | 5126 | C | GLY | 420 | 2.817 | 15.674 | 38.084 | 1.00 | 0.00 | 0.396 | 9.82 | 4.00 |
| ATOM | 5127 | O | GLY | 420 | 3.974 | 16.056 | 38.000 | 1.00 | 0.00 | −0.396 | 8.17 | −17.40 |
| ATOM | 5128 | N | ILE | 421 | 2.500 | 14.394 | 38.163 | 1.00 | 0.00 | −0.650 | 9.00 | −17.40 |
| ATOM | 5129 | HN | ILE | 421 | 1.506 | 14.123 | 38.185 | 1.00 | 0.00 | 0.440 | 0.00 | 0.00 |
| ATOM | 5130 | CA | ILE | 421 | 3.523 | 13.354 | 38.220 | 1.00 | 0.00 | 0.158 | 9.40 | 4.00 |
| ATOM | 5131 | HA | ILE | 421 | 4.380 | 13.747 | 38.766 | 1.00 | 0.00 | 0.053 | 0.00 | 0.00 |
| ATOM | 5132 | CB | ILE | 421 | 2.975 | 12.124 | 38.970 | 1.00 | 0.00 | −0.053 | 9.40 | 4.00 |
| ATOM | 5133 | HB | ILE | 421 | 2.125 | 11.694 | 38.438 | 1.00 | 0.00 | 0.053 | 0.00 | 0.00 |
| ATOM | 5134 | CG2 | ILE | 421 | 4.045 | 11.052 | 39.099 | 1.00 | 0.00 | −0.159 | 16.15 | 4.00 |
| ATOM | 5135 | HG2 | ILE | 421 | 3.637 | 10.192 | 39.631 | 1.00 | 0.00 | 0.053 | 0.00 | 0.00 |
| ATOM | 5136 | HG2 | ILE | 421 | 4.371 | 10.742 | 38.106 | 1.00 | 0.00 | 0.053 | 0.00 | 0.00 |
| ATOM | 5137 | HG2 | ILE | 421 | 4.895 | 11.451 | 39.652 | 1.00 | 0.00 | 0.053 | 0.00 | 0.00 |
| ATOM | 5138 | CG1 | ILE | 421 | 2.435 | 12.548 | 40.333 | 1.00 | 0.00 | −0.106 | 12.77 | 4.00 |
| ATOM | 5139 | HG1 | ILE | 421 | 1.944 | 13.521 | 40.307 | 1.00 | 0.00 | 0.053 | 0.00 | 0.00 |
| ATOM | 5140 | HG1 | ILE | 421 | 3.215 | 12.623 | 41.090 | 1.00 | 0.00 | 0.053 | 0.00 | 0.00 |
| ATOM | 5141 | CD1 | ILE | 421 | 1.399 | 11.567 | 40.890 | 1.00 | 0.00 | −0.159 | 16.15 | 4.00 |
| ATOM | 5142 | HD1 | ILE | 421 | 1.049 | 11.918 | 41.860 | 1.00 | 0.00 | 0.053 | 0.00 | 0.00 |
| ATOM | 5143 | HD1 | ILE | 421 | 0.555 | 11.500 | 40.203 | 1.00 | 0.00 | 0.053 | 0.00 | 0.00 |
| ATOM | 5144 | HD1 | ILE | 421 | 1.853 | 10.582 | 41.003 | 1.00 | 0.00 | 0.053 | 0.00 | 0.00 |
| ATOM | 5145 | C | ILE | 421 | 4.017 | 12.887 | 36.864 | 1.00 | 0.00 | 0.396 | 9.82 | 4.00 |
| ATOM | 5146 | O | ILE | 421 | 3.222 | 12.457 | 36.017 | 1.00 | 0.00 | −0.396 | 8.17 | −17.40 |
| ATOM | 5147 | N | VAL | 422 | 5.330 | 12.973 | 36.659 | 1.00 | 0.00 | −0.650 | 9.00 | −17.40 |
| ATOM | 5148 | HN | VAL | 422 | 5.926 | 13.379 | 37.394 | 1.00 | 0.00 | 0.440 | 0.00 | 0.00 |
| ATOM | 5149 | CA | VAL | 422 | 5.945 | 12.508 | 35.424 | 1.00 | 0.00 | 0.158 | 9.40 | 4.00 |
| ATOM | 5150 | HA | VAL | 422 | 5.120 | 12.356 | 34.727 | 1.00 | 0.00 | 0.053 | 0.00 | 0.00 |
| ATOM | 5151 | CB | VAL | 422 | 6.937 | 13.537 | 34.868 | 1.00 | 0.00 | −0.053 | 9.40 | 4.00 |
| ATOM | 5152 | HB | VAL | 422 | 7.732 | 13.718 | 35.590 | 1.00 | 0.00 | 0.053 | 0.00 | 0.00 |
| ATOM | 5153 | CG1 | VAL | 422 | 7.562 | 13.016 | 33.559 | 1.00 | 0.00 | −0.159 | 16.15 | 4.00 |
| ATOM | 5154 | HG1 | VAL | 422 | 8.264 | 13.754 | 33.172 | 1.00 | 0.00 | 0.053 | 0.00 | 0.00 |
| ATOM | 5155 | HG1 | VAL | 422 | 8.087 | 12.081 | 33.753 | 1.00 | 0.00 | 0.053 | 0.00 | 0.00 |
| ATOM | 5156 | HG1 | VAL | 422 | 6.775 | 12.843 | 32.824 | 1.00 | 0.00 | 0.053 | 0.00 | 0.00 |
| ATOM | 5157 | CG2 | VAL | 422 | 6.202 | 14.844 | 34.614 | 1.00 | 0.00 | −0.159 | 16.15 | 4.00 |
| ATOM | 5158 | HG2 | VAL | 422 | 6.899 | 15.582 | 34.218 | 1.00 | 0.00 | 0.053 | 0.00 | 0.00 |
| ATOM | 5159 | HG2 | VAL | 422 | 5.402 | 14.677 | 33.892 | 1.00 | 0.00 | 0.053 | 0.00 | 0.00 |
| ATOM | 5160 | HG2 | VAL | 422 | 5.777 | 15.210 | 35.548 | 1.00 | 0.00 | 0.053 | 0.00 | 0.00 |
| ATOM | 5161 | C | VAL | 422 | 6.669 | 11.208 | 35.791 | 1.00 | 0.00 | 0.396 | 9.82 | 4.00 |
| ATOM | 5162 | O | VAL | 422 | 7.576 | 11.199 | 36.650 | 1.00 | 0.00 | −0.396 | 8.17 | −17.40 |
| ATOM | 5163 | N | THR | 423 | 6.248 | 10.111 | 35.156 | 1.00 | 0.00 | −0.650 | 9.00 | −17.40 |
| ATOM | 5164 | HN | THR | 423 | 5.498 | 10.202 | 34.455 | 1.00 | 0.00 | 0.440 | 0.00 | 0.00 |
| ATOM | 5165 | CA | THR | 423 | 6.814 | 8.774 | 35.418 | 1.00 | 0.00 | 0.158 | 9.40 | 4.00 |
| ATOM | 5166 | HA | THR | 423 | 7.456 | 8.781 | 36.298 | 1.00 | 0.00 | 0.053 | 0.00 | 0.00 |
| ATOM | 5167 | CB | THR | 423 | 5.675 | 7.758 | 35.713 | 1.00 | 0.00 | 0.060 | 9.40 | 4.00 |
| ATOM | 5168 | HB | THR | 423 | 4.954 | 7.723 | 34.896 | 1.00 | 0.00 | 0.053 | 0.00 | 0.00 |
| ATOM | 5169 | OG1 | THR | 423 | 4.989 | 8.166 | 36.907 | 1.00 | 0.00 | −0.537 | 11.04 | −17.40 |
| ATOM | 5170 | HG1 | THR | 423 | 5.100 | 9.181 | 37.037 | 1.00 | 0.00 | 0.424 | 0.00 | 0.00 |
| ATOM | 5171 | CG2 | THR | 423 | 6.230 | 6.338 | 35.904 | 1.00 | 0.00 | −0.159 | 16.15 | 4.00 |
| ATOM | 5172 | HG2 | THR | 423 | 5.409 | 5.650 | 36.108 | 1.00 | 0.00 | 0.053 | 0.00 | 0.00 |
| ATOM | 5173 | HG2 | THR | 423 | 6.747 | 6.024 | 34.997 | 1.00 | 0.00 | 0.053 | 0.00 | 0.00 |
| ATOM | 5174 | HG2 | THR | 423 | 6.927 | 6.329 | 36.741 | 1.00 | 0.00 | 0.053 | 0.00 | 0.00 |
| ATOM | 5175 | C | THR | 423 | 7.653 | 8.286 | 34.240 | 1.00 | 0.00 | 0.396 | 9.82 | 4.00 |
| ATOM | 5176 | O | THR | 423 | 7.216 | 8.364 | 33.090 | 1.00 | 0.00 | −0.396 | 8.17 | −17.40 |
| ATOM | 5177 | N | PHE | 424 | 8.849 | 7.778 | 34.539 | 1.00 | 0.00 | −0.650 | 9.00 | −17.40 |
| ATOM | 5178 | HN | PHE | 424 | 9.129 | 7.702 | 35.527 | 1.00 | 0.00 | 0.440 | 0.00 | 0.00 |
| ATOM | 5179 | CA | PHE | 424 | 9.770 | 7.326 | 33.502 | 1.00 | 0.00 | 0.158 | 9.40 | 4.00 |
| ATOM | 5180 | HA | PHE | 424 | 9.232 | 6.783 | 32.724 | 1.00 | 0.00 | 0.053 | 0.00 | 0.00 |
| ATOM | 5181 | CB | PHE | 424 | 10.453 | 8.560 | 32.886 | 1.00 | 0.00 | −0.106 | 12.77 | 4.00 |
| ATOM | 5182 | HB1 | PHE | 424 | 9.767 | 9.217 | 32.351 | 1.00 | 0.00 | 0.053 | 0.00 | 0.00 |
| ATOM | 5183 | HB2 | PHE | 424 | 11.230 | 8.314 | 32.162 | 1.00 | 0.00 | 0.053 | 0.00 | 0.00 |
| ATOM | 5184 | CG | PHE | 424 | 11.137 | 9.454 | 33.910 | 1.00 | 0.00 | 0.000 | 7.26 | 0.60 |
| ATOM | 5185 | CD1 | PHE | 424 | 12.518 | 9.381 | 34.124 | 1.00 | 0.00 | −0.127 | 10.80 | 0.60 |
| ATOM | 5186 | HD1 | PHE | 424 | 13.116 | 8.673 | 33.550 | 1.00 | 0.00 | 0.127 | 0.00 | 0.00 |
| ATOM | 5187 | CD2 | PHE | 424 | 10.397 | 10.367 | 34.658 | 1.00 | 0.00 | −0.127 | 10.80 | 0.60 |
| ATOM | 5188 | HD2 | PHE | 424 | 9.320 | 10.437 | 34.504 | 1.00 | 0.00 | 0.127 | 0.00 | 0.00 |
| ATOM | 5189 | CE1 | PHE | 424 | 13.146 | 10.219 | 35.080 | 1.00 | 0.00 | −0.127 | 10.80 | 0.60 |
| ATOM | 5190 | HE1 | PHE | 424 | 14.222 | 10.154 | 35.236 | 1.00 | 0.00 | 0.127 | 0.00 | 0.00 |
| ATOM | 5191 | CE2 | PHE | 424 | 11.017 | 11.199 | 35.608 | 1.00 | 0.00 | −0.127 | 10.80 | 0.60 |
| ATOM | 5192 | HE2 | PHE | 424 | 10.421 | 11.907 | 36.183 | 1.00 | 0.00 | 0.127 | 0.00 | 0.00 |
| ATOM | 5193 | CZ | PHE | 424 | 12.397 | 11.116 | 35.813 | 1.00 | 0.00 | −0.127 | 10.80 | 0.60 |
| ATOM | 5194 | HZ | PHE | 424 | 12.879 | 11.758 | 36.549 | 1.00 | 0.00 | 0.127 | 0.00 | 0.00 |
| ATOM | 5195 | C | PHE | 424 | 10.822 | 6.404 | 34.090 | 1.00 | 0.00 | 0.396 | 9.82 | 4.00 |
| ATOM | 5196 | O | PHE | 424 | 10.856 | 6.196 | 35.307 | 1.00 | 0.00 | −0.396 | 8.17 | −17.40 |
| ATOM | 5197 | N | LEU | 425 | 11.664 | 5.834 | 33.232 | 1.00 | 0.00 | −0.650 | 9.00 | −17.40 |
| ATOM | 5198 | HN | LEU | 425 | 11.542 | 5.991 | 32.221 | 1.00 | 0.00 | 0.440 | 0.00 | 0.00 |
| ATOM | 5199 | CA | LEU | 425 | 12.761 | 4.987 | 33.703 | 1.00 | 0.00 | 0.158 | 9.40 | 4.00 |
| ATOM | 5200 | HA | LEU | 425 | 12.533 | 4.722 | 34.735 | 1.00 | 0.00 | 0.053 | 0.00 | 0.00 |
| ATOM | 5201 | CB | LEU | 425 | 12.891 | 3.701 | 32.861 | 1.00 | 0.00 | −0.106 | 12.77 | 4.00 |
| ATOM | 5202 | HB1 | LEU | 425 | 13.620 | 3.884 | 32.071 | 1.00 | 0.00 | 0.053 | 0.00 | 0.00 |
| ATOM | 5203 | HB2 | LEU | 425 | 11.913 | 3.471 | 32.438 | 1.00 | 0.00 | 0.053 | 0.00 | 0.00 |
| ATOM | 5204 | CG | LEU | 425 | 13.358 | 2.461 | 33.642 | 1.00 | 0.00 | −0.053 | 9.40 | 4.00 |
| ATOM | 5205 | HG | LEU | 425 | 14.264 | 2.697 | 34.199 | 1.00 | 0.00 | 0.053 | 0.00 | 0.00 |
| ATOM | 5206 | CD1 | LEU | 425 | 12.266 | 2.026 | 34.618 | 1.00 | 0.00 | −0.159 | 16.15 | 4.00 |
| ATOM | 5207 | HD1 | LEU | 425 | 12.600 | 1.147 | 35.169 | 1.00 | 0.00 | 0.053 | 0.00 | 0.00 |
| ATOM | 5208 | HD1 | LEU | 425 | 12.059 | 2.836 | 35.317 | 1.00 | 0.00 | 0.053 | 0.00 | 0.00 |
| ATOM | 5209 | HD1 | LEU | 425 | 11.358 | 1.783 | 34.064 | 1.00 | 0.00 | 0.053 | 0.00 | 0.00 |
| ATOM | 5210 | CD2 | LEU | 425 | 13.685 | 1.323 | 32.695 | 1.00 | 0.00 | −0.159 | 16.15 | 4.00 |
| ATOM | 5211 | HD2 | LEU | 425 | 14.013 | 0.455 | 33.268 | 1.00 | 0.00 | 0.053 | 0.00 | 0.00 |
| ATOM | 5212 | HD2 | LEU | 425 | 12.797 | 1.062 | 32.119 | 1.00 | 0.00 | 0.053 | 0.00 | 0.00 |
| ATOM | 5213 | HD2 | LEU | 425 | 14.480 | 1.631 | 32.016 | 1.00 | 0.00 | 0.053 | 0.00 | 0.00 |
| ATOM | 5214 | C | LEU | 425 | 14.049 | 5.825 | 33.592 | 1.00 | 0.00 | 0.396 | 9.82 | 4.00 |
| ATOM | 5215 | O | LEU | 425 | 14.235 | 6.601 | 32.629 | 1.00 | 0.00 | −0.396 | 8.17 | −17.40 |
| ATOM | 5216 | N | PHE | 426 | 14.914 | 5.708 | 34.593 | 1.00 | 0.00 | −0.650 | 9.00 | −17.40 |
| ATOM | 5217 | HN | PHE | 426 | 14.686 | 5.095 | 35.389 | 1.00 | 0.00 | 0.440 | 0.00 | 0.00 |
| ATOM | 5218 | CA | PHE | 426 | 16.184 | 6.434 | 34.584 | 1.00 | 0.00 | 0.158 | 9.40 | 4.00 |
| ATOM | 5219 | HA | PHE | 426 | 16.366 | 6.764 | 33.561 | 1.00 | 0.00 | 0.053 | 0.00 | 0.0.0 |
| ATOM | 5220 | CB | PHE | 426 | 16.156 | 7.650 | 35.517 | 1.00 | 0.00 | −0.106 | 12.77 | 4.00 |
| ATOM | 5221 | HB1 | PHE | 426 | 16.154 | 7.382 | 36.573 | 1.00 | 0.00 | 0.053 | 0.00 | 0.00 |
| ATOM | 5222 | HB2 | PHE | 426 | 15.277 | 8.280 | 35.376 | 1.00 | 0.00 | 0.053 | 0.00 | 0.00 |
| ATOM | 5223 | CG | PHE | 426 | 17.335 | 8.562 | 35.338 | 1.00 | 0.00 | 0.000 | 7.26 | 0.60 |
| ATOM | 5224 | CD1 | PHE | 426 | 17.350 | 9.514 | 34.317 | 1.00 | 0.00 | −0.127 | 10.80 | 0.60 |
| ATOM | 5225 | HD1 | PHE | 426 | 16.477 | 9.626 | 33.673 | 1.00 | 0.00 | 0.127 | 0.00 | 0.00 |
| ATOM | 5226 | CD2 | PHE | 426 | 18.461 | 8.431 | 36.153 | 1.00 | 0.00 | −0.127 | 10.80 | 0.60 |
| ATOM | 5227 | HD2 | PHE | 426 | 18.460 | 7.693 | 36.956 | 1.00 | 0.00 | 0.127 | 0.00 | 0.00 |
| ATOM | 5228 | CE1 | PHE | 426 | 18.470 | 10.325 | 34.110 | 1.00 | 0.00 | −0.127 | 10.80 | 0.60 |
| ATOM | 5229 | HE1 | PHE | 426 | 18.467 | 11.066 | 33.311 | 1.00 | 0.00 | 0.127 | 0.00 | 0.00 |
| ATOM | 5230 | CE2 | PHE | 426 | 19.588 | 9.231 | 35.954 | 1.00 | 0.00 | −0.127 | 10.80 | 0.60 |
| ATOM | 5231 | HE2 | PHE | 426 | 20.460 | 9.112 | 36.596 | 1.00 | 0.00 | 0.127 | 0.00 | 0.00 |
| ATOM | 5232 | CZ | PHE | 426 | 19.597 | 10.181 | 34.934 | 1.00 | 0.00 | −0.127 | 10.80 | 0.60 |
| ATOM | 5233 | HZ | PHE | 426 | 20.474 | 10.808 | 34.777 | 1.00 | 0.00 | 0.127 | 0.00 | 0.00 |
| ATOM | 5234 | C | PHE | 426 | 17.265 | 5.474 | 35.056 | 1.00 | 0.00 | 0.396 | 9.82 | 4.00 |
| ATOM | 5235 | O | PHE | 426 | 17.297 | 5.083 | 36.226 | 4.00 | 0.00 | −0.396 | 8.17 | −17.40 |
| ATOM | 5236 | N | ARG | 427G | 18.140 | 5.096 | 34.135 | 1.00 | 0.00 | −0.650 | 9.00 | −17.40 |
| ATOM | 5237 | HN | ARG | 427G | 18.050 | 5.470 | 33.179 | 1.00 | 0.00 | 0.440 | 0.00 | 0.00 |
| ATOM | 5238 | CA | ARG | 427G | 19.218 | 4.174 | 34.428 | 1.00 | 0.00 | 0.158 | 9.40 | 4.00 |
| ATOM | 5239 | HA | ARG | 427G | 19.726 | 3.864 | 33.514 | 1.00 | 0.00 | 0.053 | 0.00 | 0.00 |
| ATOM | 5240 | CB | ARG | 427G | 20.283 | 4.875 | 35.277 | 1.00 | 0.00 | −0.106 | 12.17 | 4.00 |
| ATOM | 5241 | HB1 | ARG | 427G | 21.160 | 4.241 | 35.407 | 1.00 | 0.00 | 0.053 | 0.00 | 0.00 |
| ATOM | 5242 | HB2 | ARG | 427G | 19.897 | 5.118 | 36.267 | 1.00 | 0.00 | 0.053 | 0.00 | 0.00 |
| ATOM | 5243 | CG | ARG | 427G | 20.736 | 6.166 | 34.626 | 1.00 | 0.00 | −0.106 | 12.77 | 4.00 |
| ATOM | 5244 | HG1 | ARG | 427G | 19.920 | 6.886 | 34.686 | 1.00 | 0.00 | 0.053 | 0.00 | 0.00 |
| ATOM | 5245 | HG2 | ARG | 427G | 20.985 | 5.957 | 33.585 | 1.00 | 0.00 | 0.053 | 0.00 | 0.00 |
| ATOM | 5246 | CD | ARG | 427G | 21.956 | 6.788 | 35.281 | 1.00 | 0.00 | 0.374 | 12.77 | 4.00 |
| ATOM | 5247 | HD1 | ARG | 427G | 21.834 | 6.931 | 36.354 | 1.00 | 0.00 | 0.053 | 0.00 | 0.00 |
| ATOM | 5248 | HD2 | ARG | 427G | 22.200 | 7.769 | 34.876 | 1.00 | 0.00 | 0.053 | 0.00 | 0.00 |
| ATOM | 5249 | NE | ARG | 427G | 23.162 | 5.979 | 35.121 | 1.00 | 0.00 | −0.819 | 9.00 | −24.67 |
| ATOM | 5250 | HE | ARG | 427G | 23.077 | 5.054 | 34.674 | 1.00 | 0.00 | 0.407 | 0.00 | 0.00 |
| ATOM | 5251 | CZ | ARG | 427G | 24.363 | 6.379 | 35.524 | 1.00 | 0.00 | 0.796 | 6.95 | 4.00 |
| ATOM | 5252 | NH1 | ARG | 427G | 24.492 | 7.562 | 36.098 | 1.00 | 0.00 | −0.746 | 9.00 | −24.67 |
| ATOM | 5253 | HH1 | ARG | 427G | 25.418 | 7.882 | 36.414 | 1.00 | 0.00 | 0.407 | 0.00 | 0.00 |
| ATOM | 5254 | HH1 | ARG | 427G | 23.666 | 8.163 | 36.229 | 1.00 | 0.00 | 0.407 | 0.00 | 0.00 |
| ATOM | 5255 | NH2 | ARG | 427G | 25.426 | 5.604 | 35.360 | 1.00 | 0.00 | −0.746 | 9.00 | −24.67 |
| ATOM | 5256 | HH2 | ARG | 427G | 26.351 | 5.925 | 35.677 | 1.00 | 0.00 | 0.407 | 0.00 | 0.00 |
| ATOM | 5257 | HH2 | ARG | 427G | 25.326 | 4.680 | 34.916 | 1.00 | 0.00 | 0.407 | 0.00 | 0.00 |
| ATOM | 5258 | C | ARG | 427G | 18.692 | 2.922 | 35.119 | 1.00 | 0.00 | 0.396 | 9.82 | 4.00 |
| ATOM | 5259 | O | ARG | 427G | 19.262 | 2.457 | 36.094 | 1.00 | 0.00 | −0.396 | 8.17 | −17.40 |
| ATOM | 5260 | N | GLY | 428 | 17.579 | 2.401 | 34.604 | 1.00 | 0.00 | −0.650 | 9.00 | −17.40 |
| ATOM | 5261 | HN | GLY | 428 | 17.133 | 2.870 | 33.802 | 1.00 | 0.00 | 0.440 | 0.00 | 0.00 |
| ATOM | 5262 | CA | GLY | 428 | 16.973 | 1.192 | 35.136 | 1.00 | 0.00 | 0.105 | 9.40 | 4.00 |
| ATOM | 5263 | HA1 | GLY | 428 | 17.777 | 0.513 | 35.418 | 1.00 | 0.00 | 0.053 | 0.00 | 0.00 |
| ATOM | 5264 | HA2 | GLY | 428 | 16.351 | 0.759 | 34.352 | 1.00 | 0.00 | 0.053 | 0.00 | 0.00 |
| ATON | 5265 | C | GLY | 428 | 16.087 | 1.360 | 36.360 | 1.00 | 0.00 | 0.396 | 9.82 | 4.00 |
| ATOM | 5266 | O | GLY | 428 | 15.515 | 0.390 | 36.859 | 1.00 | 0.00 | −0.396 | 8.17 | −17.40 |
| ATOM | 5267 | N | ARG | 429G | 15.947 | 2.591 | 36.835 | 1.00 | 0.00 | −0.650 | 9.00 | −17.40 |
| ATOM | 5268 | HN | ARG | 429G | 16.414 | 3.376 | 36.360 | 1.00 | 0.00 | 0.440 | 0.00 | 0.00 |
| ATOM | 5269 | CA | ARG | 429G | 15.137 | 2.844 | 38.023 | 1.00 | 0.00 | 0.158 | 9.40 | 4.00 |
| ATOM | 5270 | HA | ARG | 429G | 14.910 | 1.911 | 38.539 | 1.00 | 0.00 | 0.053 | 0.00 | 0.00 |
| ATOM | 5271 | CB | ARG | 429G | 15.905 | 3.752 | 38.992 | 1.00 | 0.00 | −0.106 | 12.77 | 4.00 |
| ATOM | 5272 | HB1 | ARG | 429G | 15.848 | 4.773 | 38.615 | 1.00 | 0.00 | 0.053 | 0.00 | 0.00 |
| ATOM | 5273 | HB2 | ARG | 429G | 16.938 | 3.408 | 39.029 | 1.00 | 0.00 | 0.053 | 0.00 | 0.00 |
| ATOM | 5274 | CG | ARG | 429G | 15.378 | 3.767 | 40.428 | 1.00 | 0.00 | −0.106 | 12.77 | 4.00 |
| ATOM | 5275 | HG1 | ARG | 429G | 15.778 | 2.924 | 40.992 | 1.00 | 0.00 | 0.053 | 0.00 | 0.00 |
| ATOM | 5276 | HG2 | ARG | 429G | 14.290 | 3.700 | 40.438 | 1.00 | 0.00 | 0.053 | 0.00 | 0.00 |
| ATOM | 5277 | CD | ARG | 429G | 15.787 | 5.060 | 41.139 | 1.00 | 0.00 | 0.374 | 12.77 | 4.00 |
| ATOM | 5278 | HD1 | ARG | 429G | 15.504 | 4.974 | 42.188 | 1.00 | 0.00 | 0.053 | 0.00 | 0.00 |
| ATOM | 5279 | HD2 | ARG | 429G | 15.262 | 5.889 | 40.664 | 1.00 | 0.00 | 0.053 | 0.00 | 0.00 |
| ATOM | 5280 | NE | ARG | 429G | 17.227 | 5.363 | 41.092 | 1.00 | 0.00 | −0.819 | 9.00 | −24.67 |
| ATOM | 5281 | HE | ARG | 429G | 17.583 | 5.855 | 40.260 | 1.00 | 0.00 | 0.407 | 0.00 | 0.00 |
| ATOM | 5282 | CZ | ARG | 429G | 18.105 | 5.048 | 42.045 | 1.00 | 0.00 | 0.796 | 6.95 | 4.00 |
| ATOM | 5283 | NH1 | ARG | 429G | 17.710 | 4.406 | 43.134 | 1.00 | 0.00 | −0.746 | 9.00 | −24.67 |
| ATOM | 5284 | HH1 | ARG | 429G | 18.393 | 4.165 | 43.866 | 1.00 | 0.00 | 0.407 | 0.00 | 0.00 |
| ATOM | 5285 | HH1 | ARG | 429G | 16.719 | 4.147 | 43.248 | 1.00 | 0.00 | 0.407 | 0.00 | 0.00 |
| ATOM | 5286 | NH2 | ARG | 429G | 19.380 | 5.398 | 41.922 | 1.00 | 0.00 | −0.746 | 9.00 | −24.67 |
| ATOM | 5287 | HH2 | ARG | 429G | 20.054 | 5.152 | 42.660 | 1.00 | 0.00 | 0.407 | 0.00 | 0.00 |
| ATOM | 5288 | HH2 | ARG | 429G | 19.694 | 5.914 | 41.088 | 1.00 | 0.00 | 0.407 | 0.00 | 0.00 |
| ATOM | 5289 | C | ARG | 429G | 13.820 | 3.512 | 37.643 | 1.00 | 0.00 | 0.396 | 9.82 | 4.00 |
| ATOM | 5290 | O | ARG | 429G | 13.793 | 4.400 | 36.788 | 1.00 | 0.00 | −0.396 | 8.17 | −17.40 |
| ATOM | 5291 | N | ARG | 430G | 12.733 | 3.077 | 38.264 | 1.00 | 0.00 | −0.650 | 9.00 | −17.40 |
| ATOM | 5292 | HN | ARG | 430G | 12.807 | 2.303 | 38.939 | 1.00 | 0.00 | 0.440 | 0.00 | 0.00 |
| ATOM | 5293 | CA | ARG | 430G | 11.433 | 3.681 | 38.002 | 1.00 | 0.00 | 0.158 | 9.40 | 4.00 |
| ATOM | 5294 | HA | ARG | 430G | 11.393 | 3.860 | 36.927 | 1.00 | 0.00 | 0.053 | 0.00 | 0.00 |
| ATOM | 5295 | CB | ARG | 430G | 10.295 | 2.754 | 38.437 | 1.00 | 0.00 | −0.106 | 12.77 | 4.00 |
| ATOM | 5296 | HB1 | ARG | 430G | 10.392 | 2.414 | 39.468 | 1.00 | 0.00 | 0.053 | 0.00 | 0.00 |
| ATOM | 5297 | HB2 | ARG | 430G | 10.228 | 1.848 | 37.833 | 1.00 | 0.00 | 0.053 | 0.00 | 0.00 |
| ATOM | 5298 | CG | ARG | 430G | 8.935 | 3.406 | 38.348 | 1.00 | 0.00 | −0.106 | 12.77 | 4.00 |
| ATOM | 5299 | HG1 | ARG | 430G | 8.871 | 4.257 | 39.025 | 1.00 | 0.00 | 0.053 | 0.00 | 0.00 |
| ATOM | 5300 | HG2 | ARG | 430G | 8.150 | 2.697 | 38.615 | 1.00 | 0.00 | 0.053 | 0.00 | 0.00 |
| ATOM | 5301 | CD | ARG | 430G | 8.667 | 3.904 | 36.925 | 1.00 | 0.00 | 0.374 | 12.77 | 4.00 |
| ATOM | 5302 | HD1 | ARG | 430G | 9.468 | 4.558 | 36.582 | 1.00 | 0.00 | 0.053 | 0.00 | 0.00 |
| ATOM | 5303 | HD2 | ARG | 430G | 7.733 | 4.464 | 36.876 | 1.00 | 0.00 | 0.053 | 0.00 | 0.00 |
| ATOM | 5304 | NE | ARG | 430G | 8.570 | 2.791 | 35.992 | 1.00 | 0.00 | −0.819 | 9.00 | −24.67 |
| ATOM | 5305 | HE | ARG | 430G | 8.490 | 1.842 | 36.385 | 1.00 | 0.00 | 0.407 | 0.00 | 0.00 |
| ATOM | 5306 | CZ | ARG | 430G | 8.577 | 2.912 | 34.670 | 1.00 | 0.00 | 0.796 | 6.95 | 4.00 |
| ATOM | 5307 | NH1 | ARG | 430G | 8.674 | 4.120 | 34.111 | 1.00 | 0.00 | −0.746 | 9.00 | −24.67 |
| ATOM | 5308 | HH1 | ARG | 430G | 8.679 | 4.213 | 33.085 | 1.00 | 0.00 | 0.407 | 0.00 | 0.00 |
| ATOM | 5309 | HH1 | ARG | 430G | 8.743 | 4.958 | 34.705 | 1.00 | 0.00 | 0.407 | 0.00 | 0.00 |
| ATOM | 5310 | NH2 | ARG | 430G | 8.511 | 1.814 | 33.908 | 1.00 | 0.00 | −0.746 | 9.00 | −24.67 |
| ATOM | 5311 | HH2 | ARG | 430G | 8.515 | 1.898 | 32.881 | 1.00 | 0.00 | 0.407 | 0.00 | 0.00 |
| ATOM | 5312 | HH2 | ARG | 430G | 8.455 | 0.884 | 34.348 | 1.00 | 0.00 | 0.407 | 0.00 | 0.00 |
| ATOM | 5313 | C | ARG | 430G | 11.394 | 4.970 | 38.813 | 1.00 | 0.00 | 0.396 | 9.82 | 4.00 |
| ATOM | 5314 | O | ARG | 430G | 11.573 | 4.958 | 40.045 | 1.00 | 0.00 | −0.396 | 8.17 | −17.40 |
| ATOM | 5315 | N | VAL | 431 | 11.178 | 6.082 | 38.128 | 1.00 | 0.00 | −0.650 | 9.00 | −17.40 |
| ATOM | 5316 | HN | VAL | 431 | 11.025 | 6.037 | 37.110 | 1.00 | 0.00 | 0.440 | 0.00 | 0.00 |
| ATOM | 5317 | CA | VAL | 431 | 11.155 | 7.370 | 38.811 | 1.00 | 0.00 | 0.158 | 9.40 | 4.00 |
| ATOM | 5318 | HA | VAL | 431 | 11.255 | 7.288 | 39.893 | 1.00 | 0.00 | 0.053 | 0.00 | 0.00 |
| ATOM | 5319 | CB | VAL | 431 | 12.335 | 8.254 | 38.343 | 1.00 | 0.00 | −0.053 | 9.40 | 4.00 |
| ATOM | 5320 | HB | VAL | 431 | 12.315 | 8.384 | 37.261 | 1.00 | 0.00 | 0.053 | 0.00 | 0.00 |
| ATOM | 5321 | CG1 | VAL | 431 | 12.267 | 9.638 | 38.991 | 1.00 | 0.00 | −0.159 | 16.15 | 4.00 |
| ATOM | 5322 | HG1 | VAL | 431 | 13.106 | 10.242 | 38.647 | 1.00 | 0.00 | 0.053 | 0.00 | 0.00 |
| ATOM | 5323 | HG1 | VAL | 431 | 11.332 | 10.124 | 38.713 | 1.00 | 0.00 | 0.053 | 0.00 | 0.00 |
| ATOM | 5324 | HG1 | VAL | 431 | 12.313 | 9.534 | 40.075 | 1.00 | 0.00 | 0.053 | 0.00 | 0.00 |
| ATOM | 5325 | CG2 | VAL | 431 | 13.661 | 7.566 | 38.693 | 1.00 | 0.00 | −0.159 | 16.15 | 4.00 |
| ATOM | 5326 | HG2 | VAL | 431 | 14.492 | 8.189 | 38.363 | 1.00 | 0.00 | 0.053 | 0.00 | 0.00 |
| ATOM | 5327 | HG2 | VAL | 431 | 13.722 | 7.420 | 39.771 | 1.00 | 0.00 | 0.053 | 0.00 | 0.00 |
| ATOM | 5328 | HG2 | VAL | 431 | 13.712 | 6.598 | 38.193 | 1.00 | 0.00 | 0.053 | 0.00 | 0.00 |
| ATOM | 5329 | C | VAL | 431 | 9.871 | 8.149 | 38.587 | 1.00 | 0.00 | 0.396 | 9.82 | 4.00 |
| ATOM | 5330 | O | VAL | 431 | 9.358 | 8.212 | 37.454 | 1.00 | 0.00 | −0.396 | 8.17 | −17.40 |
| ATOM | 5331 | N | HIS | 432S | 9.355 | 8.724 | 39.673 | 1.00 | 0.00 | −0.650 | 9.00 | −17.40 |
| ATOM | 5332 | HN | HIS | 432S | 9.807 | 8.561 | 40.584 | 1.00 | 0.00 | 0.440 | 0.00 | 0.00 |
| ATOM | 5333 | CA | HIS | 432S | 8.173 | 9.574 | 39.616 | 1.00 | 0.00 | 0.158 | 9.40 | 4.00 |
| ATOM | 5334 | HA | HIS | 432S | 7.799 | 9.559 | 38.592 | 1.00 | 0.00 | 0.053 | 0.00 | 0.00 |
| ATOM | 5335 | CB | HIS | 432S | 7.088 | 9.086 | 40.584 | 1.00 | 0.00 | −0.106 | 12.77 | 4.00 |
| ATOM | 5336 | HB1 | HIS | 432S | 6.213 | 9.726 | 40.471 | 1.00 | 0.00 | 0.053 | 0.00 | 0.00 |
| ATOM | 5337 | HB2 | HIS | 432S | 7.479 | 9.148 | 41.599 | 1.00 | 0.00 | 0.053 | 0.00 | 0.00 |
| ATOM | 5338 | CG | HIS | 432S | 6.655 | 7.677 | 40.348 | 1.00 | 0.00 | −0.050 | 7.26 | 0.60 |
| ATOM | 5339 | CD2 | HIS | 432S | 6.893 | 6.542 | 41.051 | 1.00 | 0.00 | −0.177 | 10.80 | 0.60 |
| ATOM | 5340 | HD2 | HIS | 432S | 7.451 | 6.463 | 41.984 | 1.00 | 0.00 | 0.127 | 0.00 | 0.00 |
| ATOM | 5341 | ND1 | HIS | 432S | 5.917 | 7.299 | 39.248 | 1.00 | 0.00 | 0.207 | 9.25 | −17.40 |
| ATOM | 5342 | HD1 | HIS | 432S | 5.574 | 7.934 | 38.513 | 1.00 | 0.00 | 0.393 | 0.00 | 0.00 |
| ATOM | 5343 | CE1 | HIS | 432S | 5.717 | 5.993 | 39.284 | 1.00 | 0.00 | −0.227 | 10.80 | 0.60 |
| ATOM | 5344 | HE1 | HIS | 432S | 5.165 | 5.413 | 38.544 | 1.00 | 0.00 | 0.127 | 0.00 | 0.00 |
| ATOM | 5345 | NE2 | HIS | 432S | 6.298 | 5.509 | 40.366 | 1.00 | 0.00 | 0.207 | 9.25 | −17.40 |
| ATOM | 5346 | HE2 | HIS | 432S | 6.304 | 4.519 | 40.650 | 1.00 | 0.00 | 0.393 | 0.00 | 0.00 |
| ATOM | 5347 | C | HIS | 432S | 8.593 | 10.994 | 40.030 | 1.00 | 0.00 | 0.396 | 9.82 | 4.00 |
| ATOM | 5348 | O | HIS | 432S | 8.753 | 11.280 | 41.226 | 1.00 | 0.00 | −0.396 | 8.17 | −17.40 |
| ATOM | 5349 | N | LEU | 433 | 8.805 | 11.873 | 39.056 | 1.00 | 0.00 | −0.650 | 9.00 | −17.40 |
| ATOM | 5350 | HN | LEU | 433 | 8.732 | 11.569 | 38.074 | 1.00 | 0.00 | 0.440 | 0.00 | 0.00 |
| ATOM | 5351 | CA | LEU | 433 | 9.139 | 13.263 | 39.356 | 1.00 | 0.00 | 0.158 | 9.40 | 4.00 |
| ATOM | 5352 | HA | LEU | 433 | 9.877 | 13.343 | 40.154 | 1.00 | 0.00 | 0.053 | 0.00 | 0.00 |
| ATOM | 5353 | CB | LEU | 433 | 9.644 | 13.996 | 38.101 | 1.00 | 0.00 | −0.106 | 12.77 | 4.00 |
| ATOM | 5354 | HB1 | LEU | 433 | 8.868 | 13.909 | 37.339 | 1.00 | 0.00 | 0.053 | 0.00 | 0.00 |
| ATOM | 5355 | HB2 | LEU | 433 | 10.566 | 13.507 | 37.788 | 1.00 | 0.00 | 0.053 | 0.00 | 0.00 |
| ATOM | 5356 | CG | LEU | 433 | 9.967 | 15.495 | 38.222 | 1.00 | 0.00 | −0.053 | 9.40 | 4.00 |
| ATOM | 5357 | HG | LEU | 433 | 9.098 | 16.044 | 38.584 | 1.00 | 0.00 | 0.053 | 0.00 | 0.00 |
| ATOM | 5358 | CD1 | LEU | 433 | 11.116 | 15.715 | 39.195 | 1.00 | 0.00 | −0.159 | 16.15 | 4.00 |
| ATOM | 5359 | HD1 | LEU | 433 | 11.331 | 16.781 | 39.268 | 1.00 | 0.00 | 0.053 | 0.00 | 0.00 |
| ATOM | 5360 | HD1 | LEU | 433 | 10.839 | 15.332 | 40.177 | 1.00 | 0.00 | 0.053 | 0.00 | 0.00 |
| ATOM | 5361 | HD1 | LEU | 433 | 12.001 | 15.189 | 38.837 | 1.00 | 0.00 | 0.053 | 0.00 | 0.00 |
| ATOM | 5362 | CD2 | LEU | 433 | 10.344 | 16.050 | 36.840 | 1.00 | 0.00 | −0.159 | 16.15 | 4.00 |
| ATOM | 5363 | HD2 | LEU | 433 | 10.573 | 17.112 | 36.924 | 1.00 | 0.00 | 0.053 | 0.00 | 0.00 |
| ATOM | 5364 | HD2 | LEU | 433 | 11.217 | 15.519 | 36.460 | 1.00 | 0.00 | 0.053 | 0.00 | 0.00 |
| ATOM | 5365 | HD2 | LEU | 433 | 9.509 | 15.912 | 36.152 | 1.00 | 0.00 | 0.053 | 0.00 | 0.00 |
| ATOM | 5366 | C | LEU | 433 | 7.759 | 13.764 | 39.756 | 1.00 | 0.00 | 0.396 | 9.82 | 4.00 |
| ATOM | 5367 | O | LEU | 433 | 6.883 | 13.927 | 38.898 | 1.00 | 0.00 | −0.396 | 8.17 | −17.40 |
| ATOM | 5368 | N | ALA | 434 | 7.558 | 14.005 | 41.052 | 1.00 | 0.00 | −0.650 | 9.00 | −17.40 |
| ATOM | 5369 | HN | ALA | 434 | 8.336 | 13.906 | 41.718 | 1.00 | 0.00 | 0.440 | 0.00 | 0.00 |
| ATOM | 5370 | CA | ALA | 434 | 6.239 | 14.408 | 41.530 | 1.00 | 0.00 | 0.158 | 9.40 | 4.00 |
| ATOM | 5371 | HA | ALA | 434 | 5.631 | 14.650 | 40.658 | 1.00 | 0.00 | 0.053 | 0.00 | 0.00 |
| ATOM | 5372 | CB | ALA | 434 | 5.569 | 13.223 | 42.231 | 1.00 | 0.00 | −0.159 | 16.15 | 4.00 |
| ATOM | 5373 | HB1 | ALA | 434 | 4.583 | 13.521 | 42.589 | 1.00 | 0.00 | 0.053 | 0.00 | 0.00 |
| ATOM | 5374 | HB2 | ALA | 434 | 5.464 | 12.396 | 41.528 | 1.00 | 0.00 | 0.053 | 0.00 | 0.00 |
| ATOM | 5375 | HB3 | ALA | 434 | 6.181 | 12.906 | 43.075 | 1.00 | 0.00 | 0.053 | 0.00 | 0.00 |
| ATOM | 5376 | C | ALA | 434 | 6.182 | 15.607 | 42.447 | 1.00 | 0.00 | 0.396 | 9.82 | 4.00 |
| ATOM | 5377 | O | ALA | 434 | 7.173 | 15.971 | 43.089 | 1.00 | 0.00 | −0.396 | 8.17 | −17.40 |
| ATOM | 5378 | N | PRO | 435 | 4.999 | 16.232 | 42.534 | 1.00 | 0.00 | −0.422 | 9.00 | −17.40 |
| ATOM | 5379 | CD | PRO | 435 | 3.769 | 16.013 | 41.742 | 1.00 | 0.00 | 0.105 | 12.77 | 4.00 |
| ATOM | 5380 | HD1 | PRO | 435 | 3.426 | 14.981 | 41.822 | 1.00 | 0.00 | 0.053 | 0.00 | 0.00 |
| ATOM | 5381 | HD2 | PRO | 435 | 3.937 | 16.224 | 40.686 | 1.00 | 0.00 | 0.053 | 0.00 | 0.00 |
| ATOM | 5382 | CA | PRO | 435 | 4.854 | 17.399 | 43.406 | 1.00 | 0.00 | 0.158 | 9.40 | 4.00 |
| ATOM | 5383 | HA | PRO | 435 | 5.824 | 17.894 | 43.395 | 1.00 | 0.00 | 0.053 | 0.00 | 0.00 |
| ATOM | 5384 | CB | PRO | 435 | 3.687 | 18.149 | 42.774 | 1.00 | 0.00 | −0.106 | 12.77 | 4.00 |
| ATOM | 5385 | HB1 | PRO | 435 | 4.008 | 18.741 | 41.917 | 1.00 | 0.00 | 0.053 | 0.00 | 0.00 |
| ATOM | 5386 | HB2 | PRO | 435 | 3.218 | 18.831 | 43.483 | 1.00 | 0.00 | 0.053 | 0.00 | 0.00 |
| ATOM | 5387 | CG | PRO | 435 | 2.775 | 17.003 | 42.379 | 1.00 | 0.00 | −0.106 | 12.77 | 4.00 |
| ATOM | 5388 | HG1 | PRO | 435 | 2.007 | 17.329 | 41.677 | 1.00 | 0.00 | 0.053 | 0.00 | 0.00 |
| ATOM | 5389 | HG2 | PRO | 435 | 2.267 | 16.581 | 43.246 | 1.00 | 0.00 | 0.053 | 0.00 | 0.00 |
| ATOM | 5390 | C | PRO | 435 | 4.483 | 16.844 | 44.772 | 1.00 | 0.00 | 0.396 | 9.82 | 4.00 |
| ATOM | 5391 | O | PRO | 435 | 4.271 | 15.629 | 44.910 | 1.00 | 0.00 | −0.396 | 8.17 | −17.40 |
| ATOM | 5392 | N | PRO | 436 | 4.390 | 17.718 | 45.797 | 1.00 | 0.00 | −0.422 | 9.00 | −17.40 |
| ATOM | 5393 | CD | PRO | 436 | 4.674 | 19.162 | 45.816 | 1.00 | 0.00 | 0.105 | 12.77 | 4.00 |
| ATOM | 5394 | HD1 | PRO | 436 | 3.798 | 19.743 | 45.525 | 1.00 | 0.00 | 0.053 | 0.00 | 0.00 |
| ATOM | 5395 | HD2 | PRO | 436 | 5.479 | 19.418 | 45.128 | 1.00 | 0.00 | 0.053 | 0.00 | 0.00 |
| ATOM | 5396 | CA | PRO | 436 | 4.024 | 17.245 | 47.139 | 1.00 | 0.00 | 0.158 | 9.40 | 4.00 |
| ATOM | 5397 | HA | PRO | 436 | 4.733 | 16.461 | 47.405 | 1.00 | 0.00 | 0.053 | 0.00 | 0.00 |
| ATOM | 5398 | CB | PRO | 436 | 4.098 | 18.506 | 47.996 | 1.00 | 0.00 | −0.106 | 12.77 | 4.00 |
| ATOM | 5399 | HB1 | PRO | 436 | 4.459 | 18.149 | 48.960 | 1.00 | 0.00 | 0.053 | 0.00 | 0.00 |
| ATOM | 5400 | HB2 | PRO | 436 | 3.073 | 18.876 | 48.010 | 1.00 | 0.00 | 0.053 | 0.00 | 0.00 |
| ATOM | 5401 | CG | PRO | 436 | 5.060 | 19.385 | 47.265 | 1.00 | 0.00 | −0.106 | 12.77 | 4.00 |
| ATOM | 5402 | HG1 | PRO | 436 | 6.091 | 19.093 | 47.464 | 1.00 | 0.00 | 0.053 | 0.00 | 0.00 |
| ATOM | 5403 | HG2 | PRO | 436 | 4.947 | 20.427 | 47.563 | 1.00 | 0.00 | 0.053 | 0.00 | 0.00 |
| ATOM | 5404 | C | PRO | 436 | 2.603 | 16.714 | 47.051 | 1.00 | 0.00 | 0.396 | 9.82 | 4.00 |
| ATOM | 5405 | O | PRO | 436 | 1.823 | 17.140 | 46.198 | 1.00 | 0.00 | −0.396 | 8.17 | −17.40 |
| ATOM | 5406 | N | GLN | 437 | 2.272 | 15.793 | 47.945 | 1.00 | 0.00 | −0.650 | 9.00 | −17.40 |
| ATOM | 5407 | HN | GLN | 437 | 2.969 | 15.516 | 48.650 | 1.00 | 0.00 | 0.440 | 0.00 | 0.00 |
| ATOM | 5408 | CA | GLN | 437 | 0.962 | 15.156 | 47.971 | 1.00 | 0.00 | 0.158 | 9.40 | 4.00 |
| ATOM | 5409 | HA | GLN | 437 | 0.821 | 14.631 | 47.026 | 1.00 | 0.00 | 0.053 | 0.00 | 0.00 |
| ATOM | 5410 | CB | GLN | 437 | 0.966 | 14.095 | 49.076 | 1.00 | 0.00 | −0.106 | 12.77 | 4.00 |
| ATOM | 5411 | HB1 | GLN | 437 | 1.097 | 14.603 | 50.031 | 1.00 | 0.00 | 0.053 | 0.00 | 0.00 |
| ATOM | 5412 | HB2 | GLN | 437 | 1.792 | 13.410 | 48.883 | 1.00 | 0.00 | 0.053 | 0.00 | 0.00 |
| ATOM | 5413 | CG | GLN | 437 | −0.284 | 13.267 | 49.179 | 1.00 | 0.00 | −0.106 | 12.77 | 4.00 |
| ATOM | 5414 | HG1 | GLN | 437 | −0.773 | 13.157 | 48.211 | 1.00 | 0.00 | 0.053 | 0.00 | 0.00 |
| ATOM | 5415 | HG2 | GLN | 437 | −1.008 | 13.716 | 49.858 | 1.00 | 0.00 | 0.053 | 0.00 | 0.00 |
| ATOM | 5416 | CD | GLN | 437 | 0.011 | 11.873 | 49.694 | 1.00 | 0.00 | 0.396 | 9.82 | 4.00 |
| ATOM | 5417 | OE1 | GLN | 437 | 0.901 | 11.687 | 50.537 | 1.00 | 0.00 | −0.396 | 8.17 | −17.40 |
| ATOM | 5418 | NE2 | GLN | 437 | −0.733 | 10.884 | 49.197 | 1.00 | 0.00 | −0.879 | 13.25 | −17.40 |
| ATOM | 5419 | HE2 | GLN | 437 | −1.461 | 11.088 | 48.497 | 1.00 | 0.00 | 0.440 | 0.00 | 0.00 |
| ATOM | 5420 | HE2 | GLN | 437 | −0.579 | 9.915 | 49.512 | 1.00 | 0.00 | 0.440 | 0.00 | 0.00 |
| ATOM | 5421 | C | GLN | 437 | −0.222 | 16.113 | 48.143 | 1.00 | 0.00 | 0.396 | 9.82 | 4.00 |
| ATOM | 5422 | O | GLN | 437 | −1.382 | 15.706 | 48.037 | 1.00 | 0.00 | −0.396 | 8.17 | −17.40 |
| ATOM | 5423 | N | THR | 438 | 0.072 | 17.381 | 48.394 | 1.00 | 0.00 | −0.650 | 9.00 | −17.40 |
| ATOM | 5424 | HN | THR | 438 | 1.059 | 17.665 | 48.467 | 1.00 | 0.00 | 0.440 | 0.00 | 0.00 |
| ATOM | 5425 | CA | THR | 438 | −0.973 | 18.380 | 48.568 | 1.00 | 0.00 | 0.158 | 9.40 | 4.00 |
| ATOM | 5426 | HA | THR | 438 | −1.874 | 17.959 | 49.015 | 1.00 | 0.00 | 0.053 | 0.00 | 0.00 |
| ATOM | 5427 | CB | THR | 438 | −0.513 | 19.488 | 49.522 | 1.00 | 0.00 | 0.060 | 9.40 | 4.00 |
| ATOM | 5428 | HB | THR | 438 | −1.232 | 20.307 | 49.528 | 1.00 | 0.00 | 0.053 | 0.00 | 0.00 |
| ATOM | 5429 | OG1 | THR | 438 | 0.751 | 19.992 | 49.083 | 1.00 | 0.00 | −0.537 | 11.04 | −17.40 |
| ATOM | 5430 | HG1 | THR | 438 | 1.397 | 20.044 | 49.883 | 1.00 | 0.00 | 0.424 | 0.00 | 0.00 |
| ATOM | 5431 | CG2 | THR | 438 | −0.382 | 18.945 | 50.948 | 1.00 | 0.00 | −0.159 | 16.15 | 4.00 |
| ATOM | 5432 | HG2 | THR | 438 | −0.054 | 19.744 | 51.613 | 1.00 | 0.00 | 0.053 | 0.00 | 0.00 |
| ATOM | 5433 | HG2 | THR | 438 | −1.347 | 18.566 | 51.283 | 1.00 | 0.00 | 0.053 | 0.00 | 0.00 |
| ATOM | 5434 | HG2 | THR | 438 | 0.349 | 18.137 | 50.964 | 1.00 | 0.00 | 0.053 | 0.00 | 0.00 |
| ATOM | 5435 | C | THR | 438 | −1.398 | 19.013 | 47.235 | 1.00 | 0.00 | 0.396 | 9.82 | 4.00 |
| ATOM | 5436 | O | THR | 438 | −2.264 | 19.894 | 47.205 | 1.00 | 0.00 | −0.396 | 8.17 | −17.40 |
| ATOM | 5437 | N | TRP | 439 | −0.783 | 18.560 | 46.143 | 1.00 | 0.00 | −0.650 | 9.00 | −17.40 |
| ATOM | 5438 | HN | TRP | 439 | −0.056 | 17.837 | 46.249 | 1.00 | 0.00 | 0.440 | 0.00 | 0.00 |
| ATOM | 5439 | CA | TRP | 439 | −1.099 | 19.048 | 44.797 | 1.00 | 0.00 | 0.158 | 9.40 | 4.00 |
| ATOM | 5440 | HA | TRP | 439 | −0.653 | 20.036 | 44.687 | 1.00 | 0.00 | 0.053 | 0.00 | 0.00 |
| ATOM | 5441 | CB | TRP | 439 | −0.576 | 18.072 | 43.744 | 1.00 | 0.00 | −0.106 | 12.77 | 4.00 |
| ATOM | 5442 | HB1 | TRP | 439 | −1.122 | 17.134 | 43.849 | 1.00 | 0.00 | 0.053 | 0.00 | 0.00 |
| ATOM | 5443 | HB2 | TRP | 439 | 0.488 | 17.920 | 43.921 | 1.00 | 0.00 | 0.053 | 0.00 | 0.00 |
| ATOM | 5444 | CG | TRP | 439 | −0.735 | 18.527 | 42.324 | 1.00 | 0.00 | 0.000 | 7.26 | 0.60 |
| ATOM | 5445 | CD2 | TRP | 439 | −1.829 | 18.243 | 41.440 | 1.00 | 0.00 | 0.000 | 6.80 | 0.60 |
| ATOM | 5446 | CE2 | TRP | 439 | −1.537 | 18.859 | 40.199 | 1.00 | 0.00 | −0.050 | 6.80 | 0.60 |
| ATOM | 5447 | CD1 | TRP | 439 | 0.147 | 19.285 | 41.605 | 1.00 | 0.00 | −0.177 | 10.80 | 0.60 |
| ATOM | 5448 | HD1 | TRP | 439 | 1.090 | 19.674 | 41.988 | 1.00 | 0.00 | 0.127 | 0.00 | 0.00 |
| ATOM | 5449 | NE1 | TRP | 439 | −0.326 | 19.487 | 40.328 | 1.00 | 0.00 | −0.292 | 9.00 | −17.40 |
| ATOM | 5450 | HE1 | TRP | 439 | 0.152 | 20.022 | 39.590 | 1.00 | 0.00 | 0.393 | 0.00 | 0.00 |
| ATOM | 5451 | CE3 | TRP | 439 | −3.025 | 17.527 | 41.573 | 1.00 | 0.00 | −0.127 | 10.80 | 0.60 |
| ATOM | 5452 | HE3 | TRP | 439 | −3.280 | 17.041 | 42.515 | 1.00 | 0.00 | 0.127 | 0.00 | 0.00 |
| ATOM | 5453 | CZ2 | TRP | 439 | −2.399 | 18.781 | 39.095 | 1.00 | 0.00 | −0.127 | 10.80 | 0.60 |
| ATOM | 5454 | HZ2 | TRP | 439 | −2.151 | 19.264 | 38.150 | 1.00 | 0.00 | 0.127 | 0.00 | 0.00 |
| ATOM | 5455 | CZ3 | TRP | 439 | −3.886 | 17.447 | 40.472 | 1.00 | 0.00 | −0.127 | 10.80 | 0.60 |
| ATOM | 5456 | HZ3 | TRP | 439 | −4.819 | 16.891 | 40.560 | 1.00 | 0.00 | 0.127 | 0.00 | 0.00 |
| ATOM | 5457 | CH2 | TRP | 439 | −3.564 | 18.074 | 39.248 | 1.00 | 0.00 | −0.127 | 10.80 | 0.60 |
| ATOM | 5458 | HH2 | TRP | 439 | −4.254 | 17.994 | 38.408 | 1.00 | 0.00 | 0.127 | 0.00 | 0.00 |
| ATOM | 5459 | C | TRP | 439 | −2.609 | 19.114 | 44.669 | 1.00 | 0.00 | 0.396 | 9.82 | 4.00 |
| ATOM | 5460 | O | TRP | 439 | −3.312 | 18.194 | 45.080 | 1.00 | 0.00 | −0.396 | 8.17 | −17.40 |
| ATOM | 5461 | N | ASP | 440P | −3.102 | 20.175 | 44.054 | 1.00 | 0.00 | −0.650 | 9.00 | −17.40 |
| ATOM | 5462 | HN | ASP | 440P | −2.461 | 20.892 | 43.685 | 1.00 | 0.00 | 0.440 | 0.00 | 0.00 |
| ATOM | 5463 | CA | ASP | 440P | −4.530 | 20.341 | 43.893 | 1.00 | 0.00 | 0.158 | 9.40 | 4.00 |
| ATOM | 5464 | HA | ASP | 440P | −5.044 | 19.381 | 43.838 | 1.00 | 0.00 | 0.053 | 0.00 | 0.00 |
| ATOM | 5465 | CB | ASP | 440P | −5.091 | 21.098 | 45.092 | 1.00 | 0.00 | −0.336 | 12.77 | 4.00 |
| ATOM | 5466 | HB1 | ASP | 440P | −4.857 | 22.162 | 45.056 | 1.00 | 0.00 | 0.053 | 0.00 | 0.00 |
| ATOM | 5467 | HB2 | ASP | 440P | −4.696 | 20.726 | 46.037 | 1.00 | 0.00 | 0.053 | 0.00 | 0.00 |
| ATOM | 5468 | CG | ASP | 440P | −6.599 | 20.992 | 45.185 | 1.00 | 0.00 | 0.297 | 9.82 | 4.00 |
| ATOM | 5469 | OD1 | ASP | 440P | −7.198 | 21.763 | 45.967 | 1.00 | 0.00 | −0.534 | 8.17 | −18.95 |
| ATOM | 5470 | OD2 | ASP | 440P | −7.183 | 20.130 | 44.483 | 1.00 | 0.00 | −0.534 | 8.17 | −18.95 |
| ATOM | 5471 | C | ASP | 440P | −4.870 | 21.107 | 42.616 | 1.00 | 0.00 | 0.396 | 9.82 | 4.00 |
| ATOM | 5472 | O | ASP | 440P | −5.526 | 22.153 | 42.671 | 1.00 | 0.00 | −0.396 | 8.17 | −17.40 |
| ATOM | 5473 | N | GLY | 441 | −4.419 | 20.607 | 41.471 | 1.00 | 0.00 | −0.650 | 9.00 | −17.40 |
| ATOM | 5474 | HN | GLY | 441 | −3.856 | 19.744 | 41.469 | 1.00 | 0.00 | 0.440 | 0.00 | 0.00 |
| ATOM | 5475 | CA | GLY | 441 | −4.723 | 21.283 | 40.221 | 1.00 | 0.00 | 0.105 | 9.40 | 4.00 |
| ATOM | 5476 | HA1 | GLY | 441 | −5.493 | 22.029 | 40.413 | 1.00 | 0.00 | 0.053 | 0.00 | 0.00 |
| ATOM | 5477 | HA2 | GLY | 441 | −5.079 | 20.540 | 39.507 | 1.00 | 0.00 | 0.053 | 0.00 | 0.00 |
| ATOM | 5478 | C | GLY | 441 | −3.536 | 21.994 | 39.595 | 1.00 | 0.00 | 0.396 | 9.82 | 4.00 |
| ATOM | 5479 | O | GLY | 441 | −2.619 | 22.453 | 40.282 | 1.00 | 0.00 | −0.396 | 8.17 | −17.40 |
| ATOM | 5480 | N | TYR | 442 | −3.565 | 22.105 | 38.273 | 1.00 | 0.00 | −0.650 | 9.00 | −17.40 |
| ATOM | 5481 | HN | TYR | 442 | −4.373 | 21.733 | 37.753 | 1.00 | 0.00 | 0.440 | 0.00 | 0.00 |
| ATOM | 5482 | CA | TYR | 442 | −2.482 | 22.739 | 37.544 | 1.00 | 0.00 | 0.158 | 9.40 | 4.00 |
| ATOM | 5483 | HA | TYR | 442 | −1.490 | 22.488 | 37.921 | 1.00 | 0.00 | 0.053 | 0.00 | 0.00 |
| ATOM | 5484 | CB | TYR | 442 | −2.489 | 22.256 | 36.094 | 1.00 | 0.00 | −0.106 | 12.77 | 4.00 |
| ATOM | 5485 | HB1 | TYR | 442 | −1.769 | 22.779 | 35.465 | 1.00 | 0.00 | 0.053 | 0.00 | 0.00 |
| ATOM | 5486 | HB2 | TYR | 442 | −3.454 | 22.384 | 35.604 | 1.00 | 0.00 | 0.053 | 0.00 | 0.00 |
| ATOM | 5487 | CG | TYR | 442 | −2.159 | 20.800 | 35.957 | 1.00 | 0.00 | 0.000 | 7.26 | 0.60 |
| ATOM | 5488 | CD1 | TYR | 442 | −3.157 | 19.858 | 35.705 | 1.00 | 0.00 | −0.127 | 10.80 | 0.60 |
| ATOM | 5489 | HD1 | TYR | 442 | −4.194 | 20.181 | 35.618 | 1.00 | 0.00 | 0.127 | 0.00 | 0.00 |
| ATOM | 5490 | CE1 | TYR | 442 | −2.841 | 18.505 | 35.563 | 1.00 | 0.00 | −0.127 | 10.80 | 0.60 |
| ATOM | 5491 | HE1 | TYR | 442 | −3.626 | 17.776 | 35.361 | 1.00 | 0.00 | 0.127 | 0.00 | 0.00 |
| ATOM | 5492 | CD2 | TYR | 442 | −0.838 | 20.357 | 36.069 | 1.00 | 0.00 | −0.127 | 10.80 | 0.60 |
| ATOM | 5493 | HD2 | TYR | 442 | −0.046 | 21.079 | 36.269 | 1.00 | 0.00 | 0.127 | 0.00 | 0.00 |
| ATOM | 5494 | CE2 | TYR | 442 | −0.517 | 19.018 | 35.930 | 1.00 | 0.00 | −0.127 | 10.80 | 0.60 |
| ATOM | 5495 | HE2 | TYR | 442 | 0.519 | 18.692 | 36.016 | 1.00 | 0.00 | 0.127 | 0.00 | 0.00 |
| ATOM | 5496 | CZ | TYR | 442 | −1.518 | 18.092 | 35.680 | 1.00 | 0.00 | 0.026 | 7.26 | 0.60 |
| ATOM | 5497 | OH | TYR | 442 | −1.207 | 16.747 | 35.580 | 1.00 | 0.00 | −0.451 | 10.94 | −17.40 |
| ATOM | 5498 | HH | TYR | 442 | −2.073 | 16.201 | 35.467 | 1.00 | 0.00 | 0.424 | 0.00 | 0.00 |
| ATOM | 5499 | C | TYR | 442 | −2.533 | 24.261 | 37.567 | 1.00 | 0.00 | 0.396 | 9.82 | 4.00 |
| ATOM | 5500 | O | TYR | 442 | −3.583 | 24.858 | 37.802 | 1.00 | 0.00 | −0.396 | 8.17 | −17.40 |
| ATOM | 5501 | N | ASP | 443P | −1.390 | 24.882 | 37.302 | 1.00 | 0.00 | −0.650 | 9.00 | −17.40 |
| ATOM | 5502 | HN | ASP | 443P | −0.547 | 24.318 | 37.116 | 1.00 | 0.00 | 0.440 | 0.00 | 0.00 |
| ATOM | 5503 | CA | ASP | 443P | −1.289 | 26.342 | 37.266 | 1.00 | 0.00 | 0.158 | 9.40 | 4.00 |
| ATOM | 5504 | HA | ASP | 443P | −2.283 | 26.755 | 37.432 | 1.00 | 0.00 | 0.053 | 0.00 | 0.00 |
| ATOM | 5505 | CB | ASP | 443P | −0.337 | 26.813 | 38.362 | 1.00 | 0.00 | −0.336 | 12.77 | 4.00 |
| ATOM | 5506 | HB1 | ASP | 443P | 0.692 | 26.502 | 38.184 | 1.00 | 0.00 | 0.053 | 0.00 | 0.00 |
| ATOM | 5507 | HB2 | ASP | 443P | −0.605 | 26.426 | 39.345 | 1.00 | 0.00 | 0.053 | 0.00 | 0.00 |
| ATOM | 5508 | CG | ASP | 443P | −0.305 | 28.316 | 38.496 | 1.00 | 0.00 | 0.297 | 9.82 | 4.00 |
| ATOM | 5509 | OD1 | ASP | 443P | −0.836 | 28.997 | 37.594 | 1.00 | 0.00 | −0.534 | 8.17 | −18.95 |
| ATOM | 5510 | OD2 | ASP | 443P | 0.253 | 28.816 | 39.496 | 1.00 | 0.00 | −0.534 | 8.17 | −18.95 |
| ATOM | 5511 | C | ASP | 443P | −0.757 | 26.748 | 35.896 | 1.00 | 0.00 | 0.396 | 9.82 | 4.00 |
| ATOM | 5512 | O | ASP | 443P | 0.424 | 26.548 | 35.601 | 1.00 | 0.00 | −0.396 | 8.17 | −17.40 |
| ATOM | 5513 | N | PRO | 444 | −1.617 | 27.340 | 35.052 | 1.00 | 0.00 | −0.422 | 9.00 | −17.40 |
| ATOM | 5514 | CD | PRO | 444 | −2.992 | 27.767 | 35.376 | 1.00 | 0.00 | 0.105 | 12.77 | 4.00 |
| ATOM | 5515 | HD1 | PRO | 444 | −3.067 | 28.107 | 36.408 | 1.00 | 0.00 | 0.053 | 0.00 | 0.00 |
| ATOM | 5516 | HD2 | PRO | 444 | −3.701 | 26.949 | 35.246 | 1.00 | 0.00 | 0.053 | 0.00 | 0.00 |
| ATOM | 5517 | CA | PRO | 444 | −1.240 | 27.777 | 33.702 | 1.00 | 0.00 | 0.158 | 9.40 | 4.00 |
| ATOM | 5518 | HA | PRO | 444 | −0.908 | 26.963 | 33.057 | 1.00 | 0.00 | 0.053 | 0.00 | 0.00 |
| ATOM | 5519 | CB | PRO | 444 | −2.547 | 28.347 | 33.153 | 1.00 | 0.00 | −0.106 | 12.77 | 4.00 |
| ATOM | 5520 | HB1 | PRO | 444 | −3.140 | 27.572 | 32.666 | 1.00 | 0.00 | 0.053 | 0.00 | 0.00 |
| ATOM | 5521 | HB2 | PRO | 444 | −2.357 | 29.129 | 32.418 | 1.00 | 0.00 | 0.053 | 0.00 | 0.00 |
| ATOM | 5522 | CG | PRO | 444 | −3.223 | 28.884 | 34.389 | 1.00 | 0.00 | −0.106 | 12.77 | 4.00 |
| ATOM | 5523 | HG1 | PRO | 444 | −4.282 | 29.071 | 34.214 | 1.00 | 0.00 | 0.053 | 0.00 | 0.00 |
| ATOM | 5524 | HG2 | PRO | 444 | −2.774 | 29.823 | 34.712 | 1.00 | 0.00 | 0.053 | 0.00 | 0.00 |
| ATOM | 5525 | C | PRO | 444 | −0.099 | 28.787 | 33.685 | 1.00 | 0.00 | 0.396 | 9.82 | 4.00 |
| ATOM | 5526 | O | PRO | 444 | 0.531 | 29.007 | 32.648 | 1.00 | 0.00 | −0.396 | 8.17 | −17.40 |
| ATOM | 5527 | N | SER | 445 | 0.184 | 29.385 | 34.838 | 1.00 | 0.00 | −0.650 | 9.00 | −17.40 |
| ATOM | 5528 | HN | SER | 445 | −0.363 | 29.147 | 35.677 | 1.00 | 0.00 | 0.440 | 0.00 | 0.00 |
| ATOM | 5529 | CA | SER | 445 | 1.257 | 30.373 | 34.938 | 1.00 | 0.00 | 0.158 | 9.40 | 4.00 |
| ATOM | 5530 | HA | SER | 445 | 1.241 | 31.064 | 34.095 | 1.00 | 0.00 | 0.053 | 0.00 | 0.00 |
| ATOM | 5531 | CB | SER | 445 | 1.079 | 31.276 | 36.183 | 1.00 | 0.00 | 0.007 | 12.77 | 4.00 |
| ATOM | 5532 | HB1 | SER | 445 | 0.117 | 31.789 | 36.199 | 1.00 | 0.00 | 0.053 | 0.00 | 0.00 |
| ATOM | 5533 | HB2 | SER | 445 | 1.836 | 32.057 | 36.250 | 1.00 | 0.00 | 0.053 | 0.00 | 0.00 |
| ATOM | 5534 | OG | SER | 445 | 1.155 | 30.556 | 37.398 | 1.00 | 0.00 | −0.537 | 11.04 | −17.40 |
| ATOM | 5535 | HG | SER | 445 | 0.679 | 31.086 | 38.142 | 1.00 | 0.00 | 0.424 | 0.00 | 0.00 |
| ATOM | 5536 | C | SER | 445 | 2.629 | 29.706 | 34.957 | 1.00 | 0.00 | 0.396 | 9.82 | 4.00 |
| ATOM | 5537 | O | SER | 445 | 3.651 | 30.395 | 34.871 | 1.00 | 0.00 | −0.396 | 8.17 | −17.40 |
| ATOM | 5538 | N | TRP | 446 | 2.652 | 28.375 | 35.081 | 1.00 | 0.00 | −0.650 | 9.00 | −17.40 |
| ATOM | 5539 | HN | TRP | 446 | 1.764 | 27.863 | 35.190 | 1.00 | 0.00 | 0.440 | 0.00 | 0.00 |
| ATOM | 5540 | CA | TRP | 446 | 3.919 | 27.625 | 35.065 | 1.00 | 0.00 | 0.158 | 9.40 | 4.00 |
| ATOM | 5541 | HA | TRP | 446 | 4.705 | 28.165 | 35.592 | 1.00 | 0.00 | 0.053 | 0.00 | 0.00 |
| ATOM | 5542 | CB | TRP | 446 | 3.758 | 26.223 | 35.685 | 1.00 | 0.00 | −0.106 | 12.77 | 4.00 |
| ATOM | 5543 | HB1 | TRP | 446 | 4.536 | 25.583 | 35.270 | 1.00 | 0.00 | 0.053 | 0.00 | 0.00 |
| ATOM | 5544 | HB2 | TRP | 446 | 2.766 | 25.854 | 35.422 | 1.00 | 0.00 | 0.053 | 0.00 | 0.00 |
| ATOM | 5545 | CG | TRP | 446 | 3.877 | 26.139 | 37.215 | 1.00 | 0.00 | 0.000 | 7.26 | 0.60 |
| ATOM | 5546 | CD2 | TRP | 446 | 4.572 | 25.126 | 37.977 | 1.00 | 0.00 | 0.000 | 6.80 | 0.60 |
| ATOM | 5547 | CE2 | TRP | 446 | 4.352 | 25.403 | 39.347 | 1.00 | 0.00 | −0.050 | 6.80 | 0.60 |
| ATOM | 5548 | CD1 | TRP | 446 | 3.286 | 26.964 | 38.138 | 1.00 | 0.00 | −0.177 | 10.80 | 0.60 |
| ATOM | 5549 | HD1 | TRP | 446 | 2.681 | 27.838 | 37.896 | 1.00 | 0.00 | 0.127 | 0.00 | 0.00 |
| ATOM | 5550 | NE1 | TRP | 446 | 3.568 | 26.525 | 39.421 | 1.00 | 0.00 | −0.292 | 9.00 | −17.40 |
| ATOM | 5551 | HE1 | TRP | 446 | 3.241 | 26.969 | 40.290 | 1.00 | 0.00 | 0.393 | 0.00 | 0.00 |
| ATOM | 5552 | CE3 | TRP | 446 | 5.354 | 24.017 | 37.631 | 1.00 | 0.00 | −0.127 | 10.80 | 0.60 |
| ATOM | 5553 | HE3 | TRP | 446 | 5.543 | 23.776 | 36.584 | 1.00 | 0.00 | 0.127 | 0.00 | 0.00 |
| ATOM | 5554 | CZ2 | TRP | 446 | 4.889 | 24.604 | 40.374 | 1.00 | 0.00 | −0.127 | 10.80 | 0.60 |
| ATOM | 5555 | HZ2 | TRP | 446 | 4.706 | 24.834 | 41.423 | 1.00 | 0.00 | 0.127 | 0.00 | 0.00 |
| ATOM | 5556 | CZ3 | TRP | 446 | 5.887 | 23.225 | 38.648 | 1.00 | 0.00 | −0.127 | 10.80 | 0.60 |
| ATOM | 5557 | HZ3 | TRP | 446 | 6.497 | 22.359 | 38.389 | 1.00 | 0.00 | 0.127 | 0.00 | 0.00 |
| ATOM | 5558 | CH2 | TRP | 446 | 5.650 | 23.525 | 40.004 | 1.00 | 0.00 | −0.127 | 10.80 | 0.60 |
| ATOM | 5559 | HH2 | TRP | 446 | 6.080 | 22.886 | 40.775 | 1.00 | 0.00 | 0.127 | 0.00 | 0.00 |
| ATOM | 5560 | C | TRP | 446 | 4.207 | 27.501 | 33.578 | 1.00 | 0.00 | 0.396 | 9.82 | 4.00 |
| ATOM | 5561 | O | TRP | 446 | 3.542 | 26.744 | 32.881 | 1.00 | 0.00 | −0.396 | 8.17 | −17.40 |
| ATOM | 5562 | N | THR | 447 | 5.180 | 28.267 | 33.087 | 1.00 | 0.00 | −0.650 | 9.00 | −17.40 |
| ATOM | 5563 | HN | THR | 447 | 5.710 | 28.881 | 33.721 | 1.00 | 0.00 | 0.440 | 0.00 | 0.00 |
| ATOM | 5564 | CA | THR | 447 | 5.506 | 28.250 | 31.668 | 1.00 | 0.00 | −0.072 | 9.40 | 4.00 |
| ATOM | 5565 | HA | THR | 447 | 4.944 | 27.476 | 31.144 | 1.00 | 0.00 | 0.053 | 0.00 | 0.00 |
| ATOM | 5566 | CB | THR | 447 | 5.173 | 29.608 | 31.001 | 1.00 | 0.00 | 0.060 | 9.40 | 4.00 |
| ATOM | 5567 | HB | THR | 447 | 5.477 | 29.586 | 29.954 | 1.00 | 0.00 | 0.053 | 0.00 | 0.00 |
| ATOM | 5568 | OG1 | THR | 447 | 5.874 | 30.663 | 31.678 | 1.00 | 0.00 | −0.537 | 11.04 | −17.40 |
| ATOM | 5569 | HG1 | THR | 447 | 6.755 | 30.863 | 31.183 | 1.00 | 0.00 | 0.424 | 0.00 | 0.00 |
| ATOM | 5570 | CG2 | THR | 447 | 3.664 | 29.893 | 31.083 | 1.00 | 0.00 | −0.159 | 16.15 | 4.00 |
| ATOM | 5571 | HG2 | THR | 447 | 3.448 | 30.850 | 30.609 | 1.00 | 0.00 | 0.053 | 0.00 | 0.00 |
| ATOM | 5572 | HG2 | THR | 447 | 3.116 | 29.102 | 30.569 | 1.00 | 0.00 | 0.053 | 0.00 | 0.00 |
| ATOM | 5573 | HG2 | THR | 447 | 3.356 | 29.927 | 32.128 | 1.00 | 0.00 | 0.053 | 0.00 | 0.00 |
| ATOM | 5574 | C | THR | 447 | 6.968 | 27.930 | 31.406 | 1.00 | 0.00 | 0.297 | 9.82 | 4.00 |
| ATOM | 5575 | O | THR | 447 | 7.719 | 27.750 | 32.375 | 1.00 | 0.00 | −0.534 | 8.17 | −17.40 |
| ATOM | 5576 | OT2 | THR | 447 | 7.336 | 27.863 | 30.218 | 1.00 | 0.00 | −0.534 | 0.00 | 0.00 |
| ATOM | 2812 | MN | MN2 | 448 | 5.281 | 16.167 | 11.610 | 1.00 | 0.00 | 0.500 | 0.00 | 0.00 |

REMARK Solvation parameters added by “addsol”

TABLE 2
REMARK Solvation parameters added by “addsol”
REMARK

| | | | | | | | | | | | | |
|---|---|---|---|---|---|---|---|---|---|---|---|---|
| ATOM | 1 | N | GLU | 325 | 5.889 | 2.827 | −27.227 | 0.00 | 0.00 | −0.247 | 9.00 | −17.40 |
| ATOM | 2 | HN1 | GLU | 325 | 5.803 | 2.916 | −28.250 | 0.00 | 0.00 | 0.280 | 0.00 | 0.00 |
| ATOM | 3 | HN2 | GLU | 325 | 6.121 | 3.745 | −26.821 | 0.00 | 0.00 | 0.280 | 0.00 | 0.00 |
| ATOM | 4 | HN3 | GLU | 325 | 6.636 | 2.155 | −27.003 | 0.00 | 0.00 | 0.280 | 0.00 | 0.00 |
| ATOM | 5 | CA | GLU | 325 | 4.664 | 2.368 | −26.689 | 0.00 | 0.00 | 0.354 | 9.40 | 4.00 |
| ATOM | 6 | HA | GLU | 325 | 3.856 | 3.064 | −26.913 | 0.00 | 0.00 | 0.053 | 0.00 | 0.00 |
| ATOM | 7 | C | GLU | 325 | 4.925 | 2.286 | −25.234 | 0.00 | 0.00 | 0.396 | 9.82 | 4.00 |
| ATOM | 8 | O | GLU | 325 | 5.847 | 2.890 | −24.693 | 0.00 | 0.00 | −0.396 | 8.17 | −17.40 |
| ATOM | 9 | CB | GLU | 325 | 4.255 | 0.957 | −27.143 | 0.00 | 0.00 | −0.106 | 12.77 | 4.00 |
| ATOM | 10 | HB1 | GLU | 325 | 3.368 | 0.660 | −26.583 | 0.00 | 0.00 | 0.053 | 0.00 | 0.00 |
| ATOM | 11 | HB2 | GLU | 325 | 5.082 | 0.277 | −26.939 | 0.00 | 0.00 | 0.053 | 0.00 | 0.00 |
| ATOM | 12 | CG | GLU | 325 | 3.923 | 0.853 | −28.631 | 0.00 | 0.00 | −0.336 | 12.77 | 4.00 |
| ATOM | 13 | HG1 | GLU | 325 | 4.763 | 1.263 | −29.192 | 0.00 | 0.00 | 0.053 | 0.00 | 0.00 |
| ATOM | 14 | HG2 | GLU | 325 | 3.015 | 1.427 | −28.813 | 0.00 | 0.00 | 0.053 | 0.00 | 0.00 |
| ATOM | 15 | CD | GLU | 325 | 3.713 | −0.619 | −28.963 | 0.00 | 0.00 | 0.297 | 9.82 | 4.00 |
| ATOM | 16 | OE1 | GLU | 325 | 3.260 | −1.374 | −28.061 | 0.00 | 0.00 | −0.534 | 8.17 | −18.95 |
| ATOM | 17 | OE2 | GLU | 325 | 4.008 | −1.007 | −30.125 | 0.00 | 0.00 | −0.534 | 8.17 | −18.95 |
| ATOM | 18 | N | ILE | 326 | 4.038 | 1.575 | −24.556 | 0.00 | 0.00 | −0.650 | 9.00 | −17.40 |
| ATOM | 19 | HN | ILE | 326 | 3.197 | 1.255 | −25.057 | 0.00 | 0.00 | 0.439 | 0.00 | 0.00 |
| ATOM | 20 | CA | ILE | 326 | 4.164 | 1.223 | −23.189 | 0.00 | 0.00 | 0.158 | 9.40 | 4.00 |
| ATOM | 21 | HA | ILE | 326 | 5.187 | 0.942 | −22.938 | 0.00 | 0.00 | 0.053 | 0.00 | 0.00 |
| ATOM | 22 | C | ILE | 326 | 3.228 | 0.096 | −23.112 | 0.00 | 0.00 | 0.396 | 9.82 | 4.00 |
| ATOM | 23 | O | ILE | 326 | 2.183 | 0.110 | −23.756 | 0.00 | 0.00 | −0.396 | 8.17 | −17.40 |
| ATOM | 24 | CB | ILE | 326 | 3.636 | 2.222 | −22.208 | 0.00 | 0.00 | −0.053 | 9.40 | 4.00 |
| ATOM | 25 | HB | ILE | 326 | 3.492 | 1.749 | −21.237 | 0.00 | 0.00 | 0.053 | 0.00 | 0.00 |
| ATOM | 26 | CG1 | ILE | 326 | 2.197 | 2.583 | −22.610 | 0.00 | 0.00 | −0.106 | 12.77 | 4.00 |
| ATOM | 27 | HG11 | ILE | 326 | 1.715 | 1.683 | −22.992 | 0.00 | 0.00 | 0.053 | 0.00 | 0.00 |
| ATOM | 28 | HG12 | ILE | 326 | 2.243 | 3.353 | −23.380 | 0.00 | 0.00 | 0.053 | 0.00 | 0.00 |
| ATOM | 29 | CG2 | ILE | 326 | 4.628 | 3.381 | −22.057 | 0.00 | 0.00 | −0.159 | 16.15 | 4.00 |
| ATOM | 30 | HG21 | ILE | 326 | 4.237 | 4.104 | −21.341 | 0.00 | 0.00 | 0.053 | 0.00 | 0.00 |
| ATOM | 31 | HG22 | ILE | 326 | 5.584 | 2.998 | −21.700 | 0.00 | 0.00 | 0.053 | 0.00 | 0.00 |
| ATOM | 32 | HG23 | ILE | 326 | 4.770 | 3.867 | −23.022 | 0.00 | 0.00 | 0.053 | 0.00 | 0.00 |
| ATOM | 33 | CD1 | ILE | 326 | 1.331 | 3.119 | −21.480 | 0.00 | 0.00 | −0.159 | 16.15 | 4.00 |
| ATOM | 34 | HD11 | ILE | 326 | 0.335 | 3.345 | −21.861 | 0.00 | 0.00 | 0.053 | 0.00 | 0.00 |
| ATOM | 35 | HD12 | ILE | 326 | 1.257 | 2.370 | −20.692 | 0.00 | 0.00 | 0.053 | 0.00 | 0.00 |
| ATOM | 36 | HD13 | ILE | 326 | 1.780 | 4.027 | −21.077 | 0.00 | 0.00 | 0.053 | 0.00 | 0.00 |
| ATOM | 37 | N | MET | 327 | 3.548 | −0.944 | −22.358 | 0.00 | 0.00 | −0.650 | 9.00 | −17.40 |
| ATOM | 38 | HN | MET | 327 | 4.434 | −1.035 | −21.841 | 0.00 | 0.00 | 0.439 | 0.00 | 0.00 |
| ATOM | 39 | CA | MET | 327 | 2.518 | −1.916 | −22.353 | 0.00 | 0.00 | 0.158 | 9.40 | 4.00 |
| ATOM | 40 | HA | MET | 327 | 1.755 | −1.687 | −23.097 | 0.00 | 0.00 | 0.053 | 0.00 | 0.00 |
| ATOM | 41 | C | MET | 327 | 1.991 | −1.822 | −20.960 | 0.00 | 0.00 | 0.396 | 9.82 | 4.00 |
| ATOM | 42 | O | MET | 327 | 2.750 | −1.601 | −20.017 | 0.00 | 0.00 | −0.396 | 8.17 | −17.40 |
| ATOM | 43 | CB | MET | 327 | 3.047 | −3.329 | −22.608 | 0.00 | 0.00 | −0.106 | 12.77 | 4.00 |
| ATOM | 44 | HB1 | MET | 327 | 3.130 | −3.826 | −21.642 | 0.00 | 0.00 | 0.053 | 0.00 | 0.00 |
| ATOM | 45 | HB2 | MET | 327 | 4.020 | −3.232 | −23.090 | 0.00 | 0.00 | 0.053 | 0.00 | 0.00 |
| ATOM | 46 | CG | MET | 327 | 2.213 | −4.249 | −23.498 | 0.00 | 0.00 | −0.041 | 12.77 | 4.00 |
| ATOM | 47 | HG1 | MET | 327 | 1.143 | −4.083 | −23.372 | 0.00 | 0.00 | 0.053 | 0.00 | 0.00 |
| ATOM | 48 | HG2 | MET | 327 | 2.393 | −5.302 | −23.284 | 0.00 | 0.00 | 0.053 | 0.00 | 0.00 |
| ATOM | 49 | SD | MET | 327 | 2.574 | −4.007 | −25.264 | 0.00 | 0.00 | −0.130 | 16.39 | −6.40 |
| ATOM | 50 | CE | MET | 327 | 1.965 | −5.631 | −25.803 | 0.00 | 0.00 | −0.094 | 16.15 | 4.00 |
| ATOM | 51 | HE1 | MET | 327 | 2.084 | −5.724 | −26.883 | 0.00 | 0.00 | 0.053 | 0.00 | 0.00 |
| ATOM | 52 | HE2 | MET | 327 | 2.535 | −6.416 | −25.306 | 0.00 | 0.00 | 0.053 | 0.00 | 0.00 |
| ATOM | 53 | HE3 | MET | 327 | 0.911 | −5.729 | −25.544 | 0.00 | 0.00 | 0.053 | 0.00 | 0.00 |
| ATOM | 54 | N | LYS | 328 | 0.661 | −1.911 | −20.804 | 0.00 | 0.00 | −0.650 | 9.00 | −17.40 |
| ATOM | 55 | HN | LYS | 328 | 0.059 | −2.026 | −21.632 | 0.00 | 0.00 | 0.439 | 0.00 | 0.00 |
| ATOM | 56 | CA | LYS | 328 | 0.068 | −1.848 | −19.505 | 0.00 | 0.00 | 0.158 | 9.40 | 4.00 |
| ATOM | 57 | HA | LYS | 328 | 0.740 | −1.223 | −18.917 | 0.00 | 0.00 | 0.053 | 0.00 | 0.00 |
| ATOM | 58 | C | LYS | 328 | 0.009 | −3.259 | −19.038 | 0.00 | 0.00 | 0.396 | 9.82 | 4.00 |
| ATOM | 59 | O | LYS | 328 | −0.599 | −4.113 | −19.684 | 0.00 | 0.00 | −0.396 | 8.17 | −17.40 |
| ATOM | 60 | CB | LYS | 328 | −1.375 | −1.325 | −19.551 | 0.00 | 0.00 | −0.106 | 12.77 | 4.00 |
| ATOM | 61 | HB1 | LYS | 328 | −1.955 | −1.989 | −20.191 | 0.00 | 0.00 | 0.053 | 0.00 | 0.00 |
| ATOM | 62 | HB2 | LYS | 328 | −1.356 | −0.314 | −19.958 | 0.00 | 0.00 | 0.053 | 0.00 | 0.00 |
| ATOM | 63 | CG | LYS | 328 | −2.086 | −1.259 | −18.199 | 0.00 | 0.00 | −0.106 | 12.77 | 4.00 |
| ATOM | 64 | HG1 | LYS | 328 | −1.521 | −0.685 | −17.464 | 0.00 | 0.00 | 0.053 | 0.00 | 0.00 |
| ATOM | 65 | HG2 | LYS | 328 | −2.244 | −2.248 | −17.768 | 0.00 | 0.00 | 0.053 | 0.00 | 0.00 |
| ATOM | 66 | CD | LYS | 328 | −3.465 | −0.601 | −18.302 | 0.00 | 0.00 | −0.106 | 12.77 | 4.00 |
| ATOM | 67 | HD1 | LYS | 328 | −3.981 | −1.021 | −19.165 | 0.00 | 0.00 | 0.053 | 0.00 | 0.00 |
| ATOM | 68 | HD2 | LYS | 328 | −3.324 | 0.473 | −18.425 | 0.00 | 0.00 | 0.053 | 0.00 | 0.00 |
| ATOM | 69 | CE | LYS | 328 | −4.356 | −0.816 | −17.077 | 0.00 | 0.00 | 0.099 | 12.77 | 4.00 |
| ATOM | 70 | HE1 | LYS | 328 | −3.858 | −1.464 | −16.356 | 0.00 | 0.00 | 0.053 | 0.00 | 0.00 |
| ATOM | 71 | HE2 | LYS | 328 | −5.297 | −1.280 | −17.371 | 0.00 | 0.00 | 0.053 | 0.00 | 0.00 |
| ATOM | 72 | NZ | LYS | 328 | −4.651 | 0.478 | −16.424 | 0.00 | 0.00 | −0.045 | 13.25 | −39.20 |
| ATOM | 73 | HZ1 | LYS | 328 | −5.251 | 0.319 | −15.602 | 0.00 | 0.00 | 0.280 | 0.00 | 0.00 |
| ATOM | 74 | HZ2 | LYS | 328 | −3.770 | 0.921 | −16.126 | 0.00 | 0.00 | 0.280 | 0.00 | 0.00 |
| ATOM | 75 | HZ3 | LYS | 328 | −5.137 | 1.096 | −17.089 | 0.00 | 0.00 | 0.280 | 0.00 | 0.00 |
| ATOM | 76 | N | LYS | 329 | 0.672 | −3.552 | −17.908 | 0.00 | 0.00 | −0.650 | 9.00 | −17.40 |
| ATOM | 77 | HN | LYS | 329 | 1.198 | −2.826 | −17.401 | 0.00 | 0.00 | 0.439 | 0.00 | 0.00 |
| ATOM | 78 | CA | LYS | 329 | 0.629 | −4.896 | −17.427 | 0.00 | 0.00 | 0.158 | 9.40 | 4.00 |
| ATOM | 79 | HA | LYS | 329 | 0.361 | −5.514 | −18.284 | 0.00 | 0.00 | 0.053 | 0.00 | 0.00 |
| ATOM | 80 | C | LYS | 329 | −0.409 | −4.917 | −16.355 | 0.00 | 0.00 | 0.396 | 9.82 | 4.00 |
| ATOM | 81 | O | LYS | 329 | −0.296 | −4.213 | −15.354 | 0.00 | 0.00 | −0.396 | 8.17 | −17.40 |
| ATOM | 82 | CB | LYS | 329 | 1.963 | −5.362 | −16.824 | 0.00 | 0.00 | −0.106 | 12.77 | 4.00 |
| ATOM | 83 | HB1 | LYS | 329 | 1.907 | −6.378 | −16.434 | 0.00 | 0.00 | 0.053 | 0.00 | 0.00 |
| ATOM | 84 | HB2 | LYS | 329 | 2.286 | −4.730 | −15.997 | 0.00 | 0.00 | 0.053 | 0.00 | 0.00 |
| ATOM | 85 | CG | LYS | 329 | 3.100 | −5.351 | −17.851 | 0.00 | 0.00 | −0.106 | 12.77 | 4.00 |
| ATOM | 86 | HG1 | LYS | 329 | 3.128 | −4.366 | −18.317 | 0.00 | 0.00 | 0.053 | 0.00 | 0.00 |
| ATOM | 87 | HG2 | LYS | 329 | 2.896 | −6.123 | −18.593 | 0.00 | 0.00 | 0.053 | 0.00 | 0.00 |
| ATOM | 88 | CD | LYS | 329 | 4.486 | −5.625 | −17.273 | 0.00 | 0.00 | −0.106 | 12.77 | 4.00 |
| ATOM | 89 | HD1 | LYS | 329 | 5.029 | −6.265 | −17.969 | 0.00 | 0.00 | 0.053 | 0.00 | 0.00 |
| ATOM | 90 | HD2 | LYS | 329 | 4.365 | −6.123 | −16.311 | 0.00 | 0.00 | 0.053 | 0.00 | 0.00 |
| ATOM | 91 | CE | LYS | 329 | 5.323 | −4.366 | −17.046 | 0.00 | 0.00 | 0.099 | 12.77 | 4.00 |
| ATOM | 92 | HE1 | LYS | 329 | 5.269 | −3.719 | −17.922 | 0.00 | 0.00 | 0.053 | 0.00 | 0.00 |
| ATOM | 93 | HE2 | LYS | 329 | 6.364 | −4.637 | −16.870 | 0.00 | 0.00 | 0.053 | 0.00 | 0.00 |
| ATOM | 94 | NZ | LYS | 329 | 4.815 | −3.625 | −15.871 | 0.00 | 0.00 | −0.045 | 13.25 | −39.20 |
| ATOM | 95 | HZ1 | LYS | 329 | 5.384 | −2.779 | −15.726 | 0.00 | 0.00 | 0.280 | 0.00 | 0.00 |
| ATOM | 96 | HZ2 | LYS | 329 | 3.834 | −3.355 | −16.033 | 0.00 | 0.00 | 0.280 | 0.00 | 0.00 |
| ATOM | 97 | HZ3 | LYS | 329 | 4.872 | −4.225 | −15.035 | 0.00 | 0.00 | 0.280 | 0.00 | 0.00 |
| ATOM | 98 | N | VAL | 330 | −1.468 | −5.726 | −16.549 | 0.00 | 0.00 | −0.650 | 9.00 | −17.40 |
| ATOM | 99 | HN | VAL | 330 | −1.528 | −6.305 | −17.398 | 0.00 | 0.00 | 0.439 | 0.00 | 0.00 |
| ATOM | 100 | CA | VAL | 330 | −2.511 | −5.777 | −15.569 | 0.00 | 0.00 | 0.158 | 9.40 | 4.00 |
| ATOM | 101 | HA | VAL | 330 | −2.417 | −4.885 | −14.949 | 0.00 | 0.00 | 0.053 | 0.00 | 0.00 |
| ATOM | 102 | C | VAL | 330 | −2.310 | −7.022 | −14.772 | 0.00 | 0.00 | 0.396 | 9.82 | 4.00 |
| ATOM | 103 | O | VAL | 330 | −2.198 | −8.117 | −15.321 | 0.00 | 0.00 | −0.396 | 8.17 | −17.40 |
| ATOM | 104 | CB | VAL | 330 | −3.891 | −5.844 | −16.166 | 0.00 | 0.00 | −0.053 | 9.40 | 4.00 |
| ATOM | 105 | HB | VAL | 330 | −3.944 | −6.698 | −16.841 | 0.00 | 0.00 | 0.053 | 0.00 | 0.00 |
| ATOM | 106 | CG1 | VAL | 330 | −4.919 | −6.005 | −15.032 | 0.00 | 0.00 | −0.159 | 16.15 | 4.00 |
| ATOM | 107 | HG11 | VAL | 330 | −5.922 | −6.055 | −15.455 | 0.00 | 0.00 | 0.053 | 0.00 | 0.00 |
| ATOM | 108 | HG12 | VAL | 330 | −4.712 | −6.922 | −14.481 | 0.00 | 0.00 | 0.053 | 0.00 | 0.00 |
| ATOM | 109 | HG13 | VAL | 330 | −4.852 | −5.152 | −14.356 | 0.00 | 0.00 | 0.053 | 0.00 | 0.00 |
| ATOM | 110 | CG2 | VAL | 330 | −4.117 | −4.588 | −17.024 | 0.00 | 0.00 | −0.159 | 16.15 | 4.00 |
| ATOM | 111 | HG21 | VAL | 330 | −5.114 | −4.622 | −17.464 | 0.00 | 0.00 | 0.053 | 0.00 | 0.00 |
| ATOM | 112 | HG22 | VAL | 330 | −4.026 | −3.699 | −16.399 | 0.00 | 0.00 | 0.053 | 0.00 | 0.00 |
| ATOM | 113 | HG23 | VAL | 330 | −3.371 | −4.551 | −17.818 | 0.00 | 0.00 | 0.053 | 0.00 | 0.00 |
| ATOM | 114 | N | VAL | 331 | −2.242 | −6.874 | −13.436 | 0.00 | 0.00 | −0.650 | 9.00 | −17.40 |
| ATOM | 115 | HN | VAL | 331 | −2.311 | −5.934 | −13.021 | 0.00 | 0.00 | 0.439 | 0.00 | 0.00 |
| ATOM | 116 | CA | VAL | 331 | −2.074 | −8.013 | −12.589 | 0.00 | 0.00 | 0.158 | 9.40 | 4.00 |
| ATOM | 117 | HA | VAL | 331 | −1.580 | −8.769 | −13.199 | 0.00 | 0.00 | 0.053 | 0.00 | 0.00 |
| ATOM | 118 | C | VAL | 331 | −3.446 | −8.416 | −12.169 | 0.00 | 0.00 | 0.396 | 9.82 | 4.00 |
| ATOM | 119 | O | VAL | 331 | −4.267 | −7.577 | −11.798 | 0.00 | 0.00 | −0.396 | 8.17 | −17.40 |
| ATOM | 120 | CB | VAL | 331 | −1.278 | −7.731 | −11.349 | 0.00 | 0.00 | −0.053 | 9.40 | 4.00 |
| ATOM | 121 | HB | VAL | 331 | −1.751 | −6.909 | −10.812 | 0.00 | 0.00 | 0.053 | 0.00 | 0.00 |
| ATOM | 122 | CG1 | VAL | 331 | −1.261 | −8.999 | −10.479 | 0.00 | 0.00 | −0.159 | 16.15 | 4.00 |
| ATOM | 123 | HG11 | VAL | 331 | −0.686 | −8.811 | −9.573 | 0.00 | 0.00 | 0.053 | 0.00 | 0.00 |
| ATOM | 124 | HG12 | VAL | 331 | −2.282 | −9.270 | −10.211 | 0.00 | 0.00 | 0.053 | 0.00 | 0.00 |
| ATOM | 125 | HG13 | VAL | 331 | −0.803 | −9.816 | −11.036 | 0.00 | 0.00 | 0.053 | 0.00 | 0.00 |
| ATOM | 126 | CG2 | VAL | 331 | 0.122 | −7.250 | −11.768 | 0.00 | 0.00 | −0.159 | 16.15 | 4.00 |
| ATOM | 127 | HG21 | VAL | 331 | 0.716 | −7.040 | −10.878 | 0.00 | 0.00 | 0.053 | 0.00 | 0.00 |
| ATOM | 128 | HG22 | VAL | 331 | 0.612 | −8.026 | −12.357 | 0.00 | 0.00 | 0.053 | 0.00 | 0.00 |
| ATOM | 129 | HG23 | VAL | 331 | 0.031 | −6.343 | −12.366 | 0.00 | 0.00 | 0.053 | 0.00 | 0.00 |
| ATOM | 130 | N | GLY | 332 | −3.739 | −9.727 | −12.240 | 0.00 | 0.00 | −0.650 | 9.00 | −17.40 |
| ATOM | 131 | HN | GLY | 332 | −3.024 | −10.407 | −12.534 | 0.00 | 0.00 | 0.439 | 0.00 | 0.00 |
| ATOM | 132 | CA | GLY | 332 | −5.059 | −10.163 | −11.901 | 0.00 | 0.00 | 0.105 | 9.40 | 4.00 |
| ATOM | 133 | HA1 | GLY | 332 | −5.574 | −10.451 | −12.817 | 0.00 | 0.00 | 0.053 | 0.00 | 0.00 |
| ATOM | 134 | HA2 | GLY | 332 | −5.581 | −9.339 | −11.414 | 0.00 | 0.00 | 0.053 | 0.00 | 0.00 |
| ATOM | 135 | C | GLY | 332 | −4.950 | −11.326 | −10.979 | 0.00 | 0.00 | 0.396 | 9.82 | 4.00 |
| ATOM | 136 | O | GLY | 332 | −3.976 | −12.080 | −11.005 | 0.00 | 0.00 | −0.396 | 8.17 | −17.40 |
| ATOM | 137 | N | ASN | 333 | −5.985 | −11.495 | −10.136 | 0.00 | 0.00 | −0.650 | 9.00 | −17.40 |
| ATOM | 138 | HN | ASN | 333 | −6.781 | −10.842 | −10.180 | 0.00 | 0.00 | 0.439 | 0.00 | 0.00 |
| ATOM | 139 | CA | ASN | 333 | −6.008 | −12.557 | −9.181 | 0.00 | 0.00 | 0.158 | 9.40 | 4.00 |
| ATOM | 140 | HA | ASN | 333 | −5.416 | −13.372 | −9.597 | 0.00 | 0.00 | 0.053 | 0.00 | 0.00 |
| ATOM | 141 | C | ASN | 333 | −7.441 | −12.948 | −8.996 | 0.00 | 0.00 | 0.396 | 9.82 | 4.00 |
| ATOM | 142 | O | ASN | 333 | −7.893 | −13.943 | −9.558 | 0.00 | 0.00 | −0.396 | 8.17 | −17.40 |
| ATOM | 143 | CB | ASN | 333 | −5.475 | −12.126 | −7.804 | 0.00 | 0.00 | −0.106 | 12.77 | 4.00 |
| ATOM | 144 | HB1 | ASN | 333 | −6.218 | −11.476 | −7.341 | 0.00 | 0.00 | 0.053 | 0.00 | 0.00 |
| ATOM | 145 | HB2 | ASN | 333 | −4.536 | −11.594 | −7.955 | 0.00 | 0.00 | 0.053 | 0.00 | 0.00 |
| ATOM | 146 | CG | ASN | 333 | −5.257 | −13.378 | −6.968 | 0.00 | 0.00 | 0.396 | 9.82 | 4.00 |
| ATOM | 147 | OD1 | ASN | 333 | −5.849 | −14.424 | −7.227 | 0.00 | 0.00 | −0.396 | 8.17 | −17.40 |
| ATOM | 148 | ND2 | ASN | 333 | −4.380 | −13.272 | −5.935 | 0.00 | 0.00 | −0.879 | 13.25 | −17.40 |
| ATOM | 149 | HD21 | ASN | 333 | −3.905 | −12.376 | −5.753 | 0.00 | 0.00 | 0.439 | 0.00 | 0.00 |
| ATOM | 150 | HD22 | ASN | 333 | −4.190 | −14.087 | −5.335 | 0.00 | 0.00 | 0.439 | 0.00 | 0.00 |
| ATOM | 151 | N | ARG | 334 | −8.201 | −12.153 | −8.213 | 0.00 | 0.00 | −0.650 | 9.00 | −17.40 |
| ATOM | 152 | HN | ARG | 334 | −7.794 | −11.295 | −7.814 | 0.00 | 0.00 | 0.439 | 0.00 | 0.00 |
| ATOM | 153 | CA | ARG | 334 | −9.567 | −12.486 | −7.928 | 0.00 | 0.00 | 0.158 | 9.40 | 4.00 |
| ATOM | 154 | HA | ARG | 334 | −9.632 | −13.566 | −7.794 | 0.00 | 0.00 | 0.053 | 0.00 | 0.00 |
| ATOM | 155 | C | ARG | 334 | −10.403 | −12.045 | −9.082 | 0.00 | 0.00 | 0.396 | 9.82 | 4.00 |
| ATOM | 156 | O | ARG | 334 | −10.533 | −10.852 | −9.351 | 0.00 | 0.00 | −0.396 | 8.17 | −17.40 |
| ATOM | 157 | CB | ARG | 334 | −10.110 | −11.768 | −6.685 | 0.00 | 0.00 | −0.106 | 12.77 | 4.00 |
| ATOM | 158 | HB1 | ARG | 334 | −11.116 | −12.084 | −6.409 | 0.00 | 0.00 | 0.053 | 0.00 | 0.00 |
| ATOM | 159 | HB2 | ARG | 334 | −10.169 | −10.686 | −6.803 | 0.00 | 0.00 | 0.053 | 0.00 | 0.00 |
| ATOM | 160 | CG | ARG | 334 | −9.258 | −11.993 | −5.435 | 0.00 | 0.00 | −0.106 | 12.77 | 4.00 |
| ATOM | 161 | HG1 | ARG | 334 | −8.220 | −11.685 | −5.560 | 0.00 | 0.00 | 0.053 | 0.00 | 0.00 |
| ATOM | 162 | HG2 | ARG | 334 | −9.215 | −13.037 | −5.125 | 0.00 | 0.00 | 0.053 | 0.00 | 0.00 |
| ATOM | 163 | CD | ARG | 334 | −9.764 | −11.227 | −4.212 | 0.00 | 0.00 | 0.374 | 12.77 | 4.00 |
| ATOM | 164 | HD1 | ARG | 334 | −9.005 | −11.318 | −3.435 | 0.00 | 0.00 | 0.053 | 0.00 | 0.00 |
| ATOM | 165 | HD2 | ARG | 334 | −10.705 | −11.686 | −3.908 | 0.00 | 0.00 | 0.053 | 0.00 | 0.00 |
| ATOM | 166 | NE | ARG | 334 | −9.956 | −9.807 | −4.626 | 0.00 | 0.00 | −0.819 | 9.00 | −24.67 |
| ATOM | 167 | HE | ARG | 334 | −10.900 | −9.470 | −4.862 | 0.00 | 0.00 | 0.407 | 0.00 | 0.00 |
| ATOM | 168 | CZ | ARG | 334 | −8.896 | −8.948 | −4.696 | 0.00 | 0.00 | 0.796 | 6.95 | 4.00 |
| ATOM | 169 | NH1 | ARG | 334 | −7.633 | −9.391 | −4.426 | 0.00 | 0.00 | −0.746 | 9.00 | −24.67 |
| ATOM | 170 | HH11 | ARG | 334 | −6.836 | −8.740 | −4.480 | 0.00 | 0.00 | 0.407 | 0.00 | 0.00 |
| ATOM | 171 | HH12 | ARG | 334 | −7.477 | −10.376 | −4.168 | 0.00 | 0.00 | 0.407 | 0.00 | 0.00 |
| ATOM | 172 | NH2 | ARG | 334 | −9.102 | −7.645 | −5.049 | 0.00 | 0.00 | −0.746 | 9.00 | −24.67 |
| ATOM | 173 | HH21 | ARG | 334 | −8.306 | −6.994 | −5.103 | 0.00 | 0.00 | 0.407 | 0.00 | 0.00 |
| ATOM | 174 | HH22 | ARG | 334 | −10.054 | −7.313 | −5.261 | 0.00 | 0.00 | 0.407 | 0.00 | 0.00 |
| ATOM | 175 | N | SER | 335 | −10.989 | −13.015 | −9.805 | 0.00 | 0.00 | −0.650 | 9.00 | −17.40 |
| ATOM | 176 | HN | SER | 335 | −10.837 | −14.004 | −9.561 | 0.00 | 0.00 | 0.439 | 0.00 | 0.00 |
| ATOM | 177 | CA | SER | 335 | −11.824 | −12.671 | −10.913 | 0.00 | 0.00 | 0.158 | 9.40 | 4.00 |
| ATOM | 178 | HA | SER | 335 | −11.235 | −12.037 | −11.576 | 0.00 | 0.00 | 0.053 | 0.00 | 0.00 |
| ATOM | 179 | C | SER | 335 | −13.001 | −11.949 | −10.368 | 0.00 | 0.00 | 0.396 | 9.32 | 4.00 |
| ATOM | 180 | O | SER | 335 | −13.358 | −10.879 | −10.859 | 0.00 | 0.00 | −0.396 | 8.17 | −17.40 |
| ATOM | 181 | CB | SER | 335 | −12.348 | −13.901 | −11.672 | 0.00 | 0.00 | 0.007 | 12.77 | 4.00 |
| ATOM | 182 | HB1 | SER | 335 | −12.935 | −14.532 | −11.005 | 0.00 | 0.00 | 0.053 | 0.00 | 0.00 |
| ATOM | 183 | HB2 | SER | 335 | −11.515 | −14.484 | −12.064 | 0.00 | 0.00 | 0.053 | 0.00 | 0.00 |
| ATOM | 184 | OG | SER | 335 | −13.170 | −13.494 | −12.756 | 0.00 | 0.00 | −0.537 | 11.04 | −17.40 |
| ATOM | 185 | HG | SER | 335 | −13.091 | −12.475 | −12.884 | 0.00 | 0.00 | 0.424 | 0.00 | 0.00 |
| ATOM | 186 | N | GLY | 336 | −13.600 | −12.511 | −9.297 | 0.00 | 0.00 | −0.650 | 9.00 | −17.40 |
| ATOM | 187 | HN | GLY | 336 | −13.203 | −13.366 | −8.882 | 0.00 | 0.00 | 0.439 | 0.00 | 0.00 |
| ATOM | 188 | CA | GLY | 336 | −14.779 | −11.932 | −8.732 | 0.00 | 0.00 | 0.105 | 9.40 | 4.00 |
| ATOM | 189 | HA1 | GLY | 336 | −14.470 | −10.957 | −8.356 | 0.00 | 0.00 | 0.053 | 0.00 | 0.00 |
| ATOM | 190 | HA2 | GLY | 336 | −15.088 | −12.619 | −7.944 | 0.00 | 0.00 | 0.053 | 0.00 | 0.00 |
| ATOM | 191 | C | GLY | 336 | −15.752 | −11.848 | −9.853 | 0.00 | 0.00 | 0.396 | 9.82 | 4.00 |
| ATOM | 192 | O | GLY | 336 | −16.373 | −12.840 | −10.236 | 0.00 | 0.00 | −0.396 | 8.17 | −17.40 |
| ATOM | 193 | N | CYS | 337 | −15.913 | −10.631 | −10.396 | 0.00 | 0.00 | −0.650 | 9.00 | −17.40 |
| ATOM | 194 | HN | CYS | 337 | −15.427 | −9.827 | −9.974 | 0.00 | 0.00 | 0.439 | 0.00 | 0.00 |
| ATOM | 195 | CA | CYS | 337 | −16.737 | −10.418 | −11.540 | 0.00 | 0.00 | 0.158 | 9.40 | 4.00 |
| ATOM | 196 | HA | CYS | 337 | −16.389 | −11.120 | −12.297 | 0.00 | 0.00 | 0.053 | 0.00 | 0.00 |
| ATOM | 197 | C | CYS | 337 | −16.530 | −8.990 | −11.939 | 0.00 | 0.00 | 0.396 | 9.82 | 4.00 |
| ATOM | 198 | O | CYS | 337 | −15.627 | −8.716 | −12.729 | 0.00 | 0.00 | −0.396 | 8.17 | −17.40 |
| ATOM | 199 | CB | CYS | 337 | −18.236 | −10.745 | −11.345 | 0.00 | 0.00 | −0.041 | 12.77 | 4.00 |
| ATOM | 200 | HB1 | CYS | 337 | −18.690 | −10.145 | −10.556 | 0.00 | 0.00 | 0.053 | 0.00 | 0.00 |
| ATOM | 201 | HB2 | CYS | 337 | −18.398 | −11.788 | −11.074 | 0.00 | 0.00 | 0.053 | 0.00 | 0.00 |
| ATOM | 202 | SG | CYS | 337 | −19.208 | −10.442 | −12.853 | 0.00 | 0.00 | −0.255 | 19.93 | −6.40 |
| ATOM | 203 | HG | CYS | 337 | −19.237 | −9.127 | −13.110 | 0.00 | 0.00 | 0.190 | 0.00 | 0.00 |
| ATOM | 204 | N | PRO | 338 | −17.314 | −8.066 | −11.440 | 0.00 | 0.00 | −0.422 | 9.00 | −17.40 |
| ATOM | 205 | CA | PRO | 338 | −17.193 | −6.706 | −11.888 | 0.00 | 0.00 | 0.158 | 9.40 | 4.00 |
| ATOM | 206 | HA | PRO | 338 | −17.424 | −6.630 | −12.950 | 0.00 | 0.00 | 0.053 | 0.00 | 0.00 |
| ATOM | 207 | CD | PRO | 338 | −17.724 | −8.098 | −10.038 | 0.00 | 0.00 | 0.105 | 12.77 | 4.00 |
| ATOM | 208 | HD1 | PRO | 338 | −16.889 | −8.538 | −9.492 | 0.00 | 0.00 | 0.053 | 0.00 | 0.00 |
| ATOM | 209 | HD2 | PRO | 338 | −18.622 | −8.714 | −10.003 | 0.00 | 0.00 | 0.053 | 0.00 | 0.00 |
| ATOM | 210 | C | PRO | 338 | −15.802 | −6.203 | −11.673 | 0.00 | 0.00 | 0.396 | 9.82 | 4.00 |
| ATOM | 211 | O | PRO | 338 | −15.308 | −5.431 | −12.492 | 0.00 | 0.00 | −0.396 | 8.17 | −17.40 |
| ATOM | 212 | CB | PRO | 338 | −18.133 | −5.912 | −10.991 | 0.00 | 0.00 | −0.106 | 12.77 | 4.00 |
| ATOM | 213 | HB1 | PRO | 338 | −19.156 | −5.940 | −11.366 | 0.00 | 0.00 | 0.053 | 0.00 | 0.00 |
| ATOM | 214 | HB2 | PRO | 338 | −17.833 | −4.866 | −10.930 | 0.00 | 0.00 | 0.053 | 0.00 | 0.00 |
| ATOM | 215 | CG | PRO | 338 | −17.989 | −6.637 | −9.643 | 0.00 | 0.00 | −0.106 | 12.77 | 4.00 |
| ATOM | 216 | HG1 | PRO | 338 | −18.899 | −6.540 | −9.050 | 0.00 | 0.00 | 0.053 | 0.00 | 0.00 |
| ATOM | 217 | HG2 | PRO | 338 | −17.164 | −6.222 | −9.064 | 0.00 | 0.00 | 0.053 | 0.00 | 0.00 |
| ATOM | 218 | N | THR | 339 | −15.145 | −6.630 | −10.580 | 0.00 | 0.00 | −0.650 | 9.00 | −17.40 |
| ATOM | 219 | HN | THR | 339 | −15.539 | −7.380 | −9.994 | 0.00 | 0.00 | 0.439 | 0.00 | 0.00 |
| ATOM | 220 | CA | THR | 339 | −13.892 | −6.029 | −10.243 | 0.00 | 0.00 | 0.158 | 9.40 | 4.00 |
| ATOM | 221 | HA | THR | 339 | −13.989 | −4.959 | −10.059 | 0.00 | 0.00 | 0.053 | 0.00 | 0.00 |
| ATOM | 222 | C | THR | 339 | −12.881 | −6.191 | −11.334 | 0.00 | 0.00 | 0.396 | 9.82 | 4.00 |
| ATOM | 223 | O | THR | 339 | −12.377 | −5.197 | −11.853 | 0.00 | 0.00 | −0.396 | 8.17 | −17.40 |
| ATOM | 224 | CB | THR | 339 | −13.334 | −6.570 | −8.956 | 0.00 | 0.00 | 0.060 | 9.40 | 4.00 |
| ATOM | 225 | HB | THR | 339 | −14.012 | −6.335 | −8.136 | 0.00 | 0.00 | 0.053 | 0.00 | 0.00 |
| ATOM | 226 | OG1 | THR | 339 | −12.095 | −5.945 | −8.652 | 0.00 | 0.00 | −0.537 | 11.04 | −17.40 |
| ATOM | 227 | HG1 | THR | 339 | −11.368 | −6.293 | −9.293 | 0.00 | 0.00 | 0.424 | 0.00 | 0.00 |
| ATOM | 228 | CG2 | THR | 339 | −13.175 | −8.096 | −9.064 | 0.00 | 0.00 | −0.159 | 16.15 | 4.00 |
| ATOM | 229 | HG21 | THR | 339 | −12.770 | −8.487 | −8.130 | 0.00 | 0.00 | 0.053 | 0.00 | 0.00 |
| ATOM | 230 | HG22 | THR | 339 | −14.147 | −8.550 | −9.257 | 0.00 | 0.00 | 0.053 | 0.00 | 0.00 |
| ATOM | 231 | HG23 | THR | 339 | −12.495 | −8.334 | −9.882 | 0.00 | 0.00 | 0.053 | 0.00 | 0.00 |
| ATOM | 232 | N | VAL | 340 | −12.555 | −7.430 | −11.737 | 0.00 | 0.00 | −0.650 | 9.00 | −17.40 |
| ATOM | 233 | HN | VAL | 340 | −12.998 | −8.260 | −11.318 | 0.00 | 0.00 | 0.439 | 0.00 | 0.00 |
| ATOM | 234 | CA | VAL | 340 | −11.570 | −7.559 | −12.770 | 0.00 | 0.00 | 0.158 | 9.40 | 4.00 |
| ATOM | 235 | HA | VAL | 340 | −10.686 | −7.001 | −12.462 | 0.00 | 0.00 | 0.053 | 0.00 | 0.00 |
| ATOM | 236 | C | VAL | 340 | −12.145 | −6.997 | −14.025 | 0.00 | 0.00 | 0.396 | 9.82 | 4.00 |
| ATOM | 237 | O | VAL | 340 | −11.651 | −6.018 | −14.582 | 0.00 | 0.00 | −0.396 | 8.17 | −17.40 |
| ATOM | 238 | CB | VAL | 340 | −11.288 | −9.002 | −13.089 | 0.00 | 0.00 | −0.053 | 9.40 | 4.00 |
| ATOM | 239 | HB | VAL | 340 | −12.220 | −9.507 | −13.342 | 0.00 | 0.00 | 0.053 | 0.00 | 0.00 |
| ATOM | 240 | CG1 | VAL | 340 | −10.323 | −9.083 | −14.279 | 0.00 | 0.00 | −0.159 | 16.15 | 4.00 |
| ATOM | 241 | HG11 | VAL | 340 | −10.118 | −10.128 | −14.511 | 0.00 | 0.00 | 0.053 | 0.00 | 0.00 |
| ATOM | 242 | HG12 | VAL | 340 | −10.774 | −8.601 | −15.146 | 0.00 | 0.00 | 0.053 | 0.00 | 0.00 |
| ATOM | 243 | HG13 | VAL | 340 | −9.391 | −8.578 | −14.026 | 0.00 | 0.00 | 0.053 | 0.00 | 0.00 |
| ATOM | 244 | CG2 | VAL | 340 | −10.783 | −9.715 | −11.832 | 0.00 | 0.00 | −0.159 | 16.15 | 4.00 |
| ATOM | 245 | HG21 | VAL | 340 | −10.579 | −10.760 | −12.064 | 0.00 | 0.00 | 0.053 | 0.00 | 0.00 |
| ATOM | 246 | HG22 | VAL | 340 | −9.868 | −9.235 | −11.484 | 0.00 | 0.00 | 0.053 | 0.00 | 0.00 |
| ATOM | 247 | HG23 | VAL | 340 | −11.542 | −9.658 | −11.052 | 0.00 | 0.00 | 0.053 | 0.00 | 0.00 |
| ATOM | 248 | N | GLY | 341 | −13.265 | −7.598 | −14.463 | 0.00 | 0.00 | −0.650 | 9.00 | −17.40 |
| ATOM | 249 | HN | GLY | 341 | −13.745 | −8.291 | −13.871 | 0.00 | 0.00 | 0.439 | 0.00 | 0.00 |
| ATOM | 250 | CA | GLY | 341 | −13.787 | −7.273 | −15.752 | 0.00 | 0.00 | 0.105 | 9.40 | 4.00 |
| ATOM | 251 | HA1 | GLY | 341 | −14.618 | −7.907 | −16.062 | 0.00 | 0.00 | 0.053 | 0.00 | 0.00 |
| ATOM | 252 | HA2 | GLY | 341 | −13.056 | −7.358 | −16.556 | 0.00 | 0.00 | 0.053 | 0.00 | 0.00 |
| ATOM | 253 | C | GLY | 341 | −14.308 | −5.883 | −15.855 | 0.00 | 0.00 | 0.396 | 9.82 | 4.00 |
| ATOM | 254 | O | GLY | 341 | −13.731 | −5.065 | −16.570 | 0.00 | 0.00 | −0.396 | 8.17 | −17.40 |
| ATOM | 255 | N | ASP | 342 | −15.382 | −5.553 | −15.109 | 0.00 | 0.00 | −0.650 | 9.00 | −17.40 |
| ATOM | 256 | HN | ASP | 342 | −15.758 | −6.179 | −14.383 | 0.00 | 0.00 | 0.439 | 0.00 | 0.00 |
| ATOM | 257 | CA | ASP | 342 | −15.966 | −4.278 | −15.390 | 0.00 | 0.00 | 0.158 | 9.40 | 4.00 |
| ATOM | 258 | HA | ASP | 342 | −15.892 | −4.075 | −16.458 | 0.00 | 0.00 | 0.053 | 0.00 | 0.00 |
| ATOM | 259 | C | ASP | 342 | −15.243 | −3.230 | −14.633 | 0.00 | 0.00 | 0.396 | 9.82 | 4.00 |
| ATOM | 260 | O | ASP | 342 | −15.832 | −2.286 | −14.108 | 0.00 | 0.00 | −0.396 | 8.17 | −17.40 |
| ATOM | 261 | CB | ASP | 342 | −17.499 | −4.187 | −15.169 | 0.00 | 0.00 | −0.336 | 12.77 | 4.00 |
| ATOM | 262 | HB1 | ASP | 342 | −17.978 | −4.952 | −15.780 | 0.00 | 0.00 | 0.053 | 0.00 | 0.00 |
| ATOM | 263 | HB2 | ASP | 342 | −17.829 | −3.192 | −15.469 | 0.00 | 0.00 | 0.053 | 0.00 | 0.00 |
| ATOM | 264 | CG | ASP | 342 | −17.941 | −4.404 | −13.729 | 0.00 | 0.00 | 0.297 | 9.82 | 4.00 |
| ATOM | 265 | OD1 | ASP | 342 | −17.289 | −3.882 | −12.788 | 0.00 | 0.00 | −0.534 | 8.17 | −18.95 |
| ATOM | 266 | OD2 | ASP | 342 | −18.974 | −5.106 | −13.562 | 0.00 | 0.00 | −0.534 | 8.17 | −18.95 |
| ATOM | 267 | N | ARG | 343 | −13.911 | −3.375 | −14.583 | 0.00 | 0.00 | −0.650 | 9.00 | −17.40 |
| ATOM | 268 | HN | ARG | 343 | −13.465 | −4.208 | −14.992 | 0.00 | 0.00 | 0.439 | 0.00 | 0.00 |
| ATOM | 269 | CA | ARG | 343 | −13.119 | −2.373 | −13.963 | 0.00 | 0.00 | 0.158 | 9.40 | 4.00 |
| ATOM | 270 | HA | ARG | 343 | −13.772 | −1.888 | −13.237 | 0.00 | 0.00 | 0.053 | 0.00 | 0.00 |
| ATOM | 271 | C | ARG | 343 | −12.690 | −1.459 | −15.063 | 0.00 | 0.00 | 0.396 | 9.82 | 4.00 |
| ATOM | 272 | O | ARG | 343 | −12.634 | −0.246 | −14.879 | 0.00 | 0.00 | −0.396 | 8.17 | −17.40 |
| ATOM | 273 | CB | ARG | 343 | −11.844 | −2.932 | −13.305 | 0.00 | 0.00 | −0.106 | 12.77 | 4.00 |
| ATOM | 274 | HB1 | ARG | 343 | −11.084 | −3.239 | −14.024 | 0.00 | 0.00 | 0.053 | 0.00 | 0.00 |
| ATOM | 275 | HB2 | ARG | 343 | −12.025 | −3.811 | −12.687 | 0.00 | 0.00 | 0.053 | 0.00 | 0.00 |
| ATOM | 276 | CG | ARG | 343 | −11.145 | −1.930 | −12.384 | 0.00 | 0.00 | −0.106 | 12.77 | 4.00 |
| ATOM | 277 | HG1 | ARG | 343 | −11.110 | −0.967 | −12.893 | 0.00 | 0.00 | 0.053 | 0.00 | 0.00 |
| ATOM | 278 | HG2 | ARG | 343 | −10.138 | −2.297 | −12.183 | 0.00 | 0.00 | 0.053 | 0.00 | 0.00 |
| ATOM | 279 | CD | ARG | 343 | −11.836 | −1.714 | −11.032 | 0.00 | 0.00 | 0.374 | 12.77 | 4.00 |
| ATOM | 280 | HD1 | ARG | 343 | −11.189 | −1.108 | −10.398 | 0.00 | 0.00 | 0.053 | 0.00 | 0.00 |
| ATOM | 281 | HD2 | ARG | 343 | −12.013 | −2.684 | −10.567 | 0.00 | 0.00 | 0.053 | 0.00 | 0.00 |
| ATOM | 282 | NE | ARG | 343 | −13.133 | −1.011 | −11.259 | 0.00 | 0.00 | −0.819 | 9.00 | −24.67 |
| ATOM | 283 | HE | ARG | 343 | −13.146 | 0.015 | −11.347 | 0.00 | 0.00 | 0.407 | 0.00 | 0.00 |
| ATOM | 284 | CZ | ARG | 343 | −14.302 | −1.711 | −11.351 | 0.00 | 0.00 | 0.796 | 6.95 | 4.00 |
| ATOM | 285 | NH1 | ARG | 343 | −14.297 | −3.070 | −11.230 | 0.00 | 0.00 | −0.746 | 9.00 | −24.67 |
| ATOM | 286 | HH11 | ARG | 343 | −15.180 | −3.596 | −11.300 | 0.00 | 0.00 | 0.407 | 0.00 | 0.00 |
| ATOM | 287 | HH12 | ARG | 343 | −13.411 | −3.569 | −11.069 | 0.00 | 0.00 | 0.407 | 0.00 | 0.00 |
| ATOM | 288 | NH2 | ARG | 343 | −15.476 | −1.049 | −11.567 | 0.00 | 0.00 | −0.746 | 9.00 | −24.67 |
| ATOM | 289 | HH21 | ARG | 343 | −16.359 | −1.575 | −11.637 | 0.00 | 0.00 | 0.407 | 0.00 | 0.00 |
| ATOM | 290 | HH22 | ARG | 343 | −15.479 | −0.023 | −11.660 | 0.00 | 0.00 | 0.407 | 0.00 | 0.00 |
| ATOM | 291 | N | ILE | 344 | −12.440 | −2.035 | −16.262 | 0.00 | 0.00 | −0.650 | 9.00 | −17.40 |
| ATOM | 292 | HN | ILE | 344 | −12.563 | −3.053 | −16.354 | 0.00 | 0.00 | 0.439 | 0.00 | 0.00 |
| ATOM | 293 | CA | ILE | 344 | −12.012 | −1.290 | −17.408 | 0.00 | 0.00 | 0.158 | 9.40 | 4.00 |
| ATOM | 294 | HA | ILE | 344 | −12.459 | −0.296 | −17.388 | 0.00 | 0.00 | 0.053 | 0.00 | 0.00 |
| ATOM | 295 | C | ILE | 344 | −12.441 | −2.003 | −18.669 | 0.00 | 0.00 | 0.396 | 9.82 | 4.00 |
| ATOM | 296 | O | ILE | 344 | −12.607 | −3.217 | −18.642 | 0.00 | 0.00 | −0.396 | 8.17 | −17.40 |
| ATOM | 297 | CB | ILE | 344 | −10.506 | −1.134 | −17.348 | 0.00 | 0.00 | −0.053 | 9.40 | 4.00 |
| ATOM | 298 | HB | ILE | 344 | −10.200 | −0.636 | −18.268 | 0.00 | 0.00 | 0.053 | 0.00 | 0.00 |
| ATOM | 299 | CG1 | ILE | 344 | −9.803 | −2.485 | −17.271 | 0.00 | 0.00 | −0.106 | 12.77 | 4.00 |
| ATOM | 300 | HG11 | ILE | 344 | −10.151 | −3.205 | −18.011 | 0.00 | 0.00 | 0.053 | 0.00 | 0.00 |
| ATOM | 301 | HG12 | ILE | 344 | −9.924 | −2.990 | −16.313 | 0.00 | 0.00 | 0.053 | 0.00 | 0.00 |
| ATOM | 302 | CG2 | ILE | 344 | −10.192 | −0.287 | −16.110 | 0.00 | 0.00 | −0.159 | 16.15 | 4.00 |
| ATOM | 303 | HG21 | ILE | 344 | −9.114 | −0.149 | −16.027 | 0.00 | 0.00 | 0.053 | 0.00 | 0.00 |
| ATOM | 304 | HG22 | ILE | 344 | −10.676 | 0.685 | −16.202 | 0.00 | 0.00 | 0.053 | 0.00 | 0.00 |
| ATOM | 305 | HG23 | ILE | 344 | −10.562 | −0.794 | −15.219 | 0.00 | 0.00 | 0.053 | 0.00 | 0.00 |
| ATOM | 306 | CD1 | ILE | 344 | −8.293 | −2.407 | −17.483 | 0.00 | 0.00 | −0.159 | 16.15 | 4.00 |
| ATOM | 307 | HD11 | ILE | 344 | −7.864 | −3.407 | −17.414 | 0.00 | 0.00 | 0.053 | 0.00 | 0.00 |
| ATOM | 308 | HD12 | ILE | 344 | −8.085 | −1.990 | −18.468 | 0.00 | 0.00 | 0.053 | 0.00 | 0.00 |
| ATOM | 309 | HD13 | ILE | 344 | −7.851 | −1.769 | −16.718 | 0.00 | 0.00 | 0.053 | 0.00 | 0.00 |
| ATOM | 310 | N | VAL | 345 | −12.690 | −1.280 | −19.800 | 0.00 | 0.00 | −0.650 | 9.00 | −17.40 |
| ATOM | 311 | HN | VAL | 345 | −12.586 | −0.255 | −19.808 | 0.00 | 0.00 | 0.439 | 0.00 | 0.00 |
| ATOM | 312 | CA | VAL | 345 | −13.105 | −1.998 | −20.988 | 0.00 | 0.00 | 0.158 | 9.40 | 4.00 |
| ATOM | 313 | HA | VAL | 345 | −13.852 | −2.708 | −20.633 | 0.00 | 0.00 | 0.053 | 0.00 | 0.00 |
| ATOM | 314 | C | VAL | 345 | −11.857 | −2.638 | −21.496 | 0.00 | 0.00 | 0.396 | 9.82 | 4.00 |
| ATOM | 315 | O | VAL | 345 | −11.894 | −3.537 | −22.335 | 0.00 | 0.00 | −0.396 | 8.17 | −17.40 |
| ATOM | 316 | CB | VAL | 345 | −13.636 | −1.263 | −22.215 | 0.00 | 0.00 | −0.053 | 9.40 | 4.00 |
| ATOM | 317 | HB | VAL | 345 | −14.156 | −1.982 | −22.848 | 0.00 | 0.00 | 0.053 | 0.00 | 0.00 |
| ATOM | 318 | CG1 | VAL | 345 | −14.616 | −0.134 | −21.884 | 0.00 | 0.00 | −0.159 | 16.15 | 4.00 |
| ATOM | 319 | HG11 | VAL | 345 | −14.949 | 0.341 | −22.807 | 0.00 | 0.00 | 0.053 | 0.00 | 0.00 |
| ATOM | 320 | HG12 | VAL | 345 | −15.477 | −0.543 | −21.355 | 0.00 | 0.00 | 0.053 | 0.00 | 0.00 |
| ATOM | 321 | HG13 | VAL | 345 | −14.121 | 0.605 | −21.254 | 0.00 | 0.00 | 0.053 | 0.00 | 0.00 |
| ATOM | 322 | CG2 | VAL | 345 | −12.506 | −0.921 | −23.176 | 0.00 | 0.00 | −0.159 | 16.15 | 4.00 |
| ATOM | 323 | HG21 | VAL | 345 | −12.911 | −0.398 | −24.042 | 0.00 | 0.00 | 0.053 | 0.00 | 0.00 |
| ATOM | 324 | HG22 | VAL | 345 | −11.781 | −0.282 | −22.672 | 0.00 | 0.00 | 0.053 | 0.00 | 0.00 |
| ATOM | 325 | HG23 | VAL | 345 | −12.016 | −1.838 | −23.503 | 0.00 | 0.00 | 0.053 | 0.00 | 0.00 |
| ATOM | 326 | N | GLU | 346 | −10.691 | −2.122 | −21.067 | 0.00 | 0.00 | −0.650 | 9.00 | −17.40 |
| ATOM | 327 | HN | GLU | 346 | −10.636 | −1.267 | −20.495 | 0.00 | 0.00 | 0.439 | 0.00 | 0.00 |
| ATOM | 328 | CA | GLU | 346 | −9.542 | −2.853 | −21.468 | 0.00 | 0.00 | 0.158 | 9.40 | 4.00 |
| ATOM | 329 | HA | GLU | 346 | −9.610 | −2.878 | −22.556 | 0.00 | 0.00 | 0.053 | 0.00 | 0.00 |
| ATOM | 330 | C | GLU | 346 | −9.709 | −4.171 | −20.817 | 0.00 | 0.00 | 0.396 | 9.82 | 4.00 |
| ATOM | 331 | O | GLU | 346 | −9.628 | −5.209 | −21.471 | 0.00 | 0.00 | −0.396 | 8.17 | −17.40 |
| ATOM | 332 | CB | GLU | 346 | −8.193 | −2.317 | −20.949 | 0.00 | 0.00 | −0.106 | 12.77 | 4.00 |
| ATOM | 333 | HB1 | GLU | 346 | −7.488 | −3.148 | −20.933 | 0.00 | 0.00 | 0.053 | 0.00 | 0.00 |
| ATOM | 334 | HB2 | GLU | 346 | −8.355 | −1.921 | −19.946 | 0.00 | 0.00 | 0.053 | 0.00 | 0.00 |
| ATOM | 335 | CG | GLU | 346 | −7.548 | −1.201 | −21.770 | 0.00 | 0.00 | −0.336 | 12.77 | 4.00 |
| ATOM | 336 | HG1 | GLU | 346 | −6.685 | −0.822 | −21.223 | 0.00 | 0.00 | 0.053 | 0.00 | 0.00 |
| ATOM | 337 | HG2 | GLU | 346 | −8.282 | −0.408 | −21.916 | 0.00 | 0.00 | 0.053 | 0.00 | 0.00 |
| ATOM | 338 | CD | GLU | 346 | −7.115 | −1.774 | −23.111 | 0.00 | 0.00 | 0.297 | 9.82 | 4.00 |
| ATOM | 339 | OE1 | GLU | 346 | −7.943 | −2.477 | −23.747 | 0.00 | 0.00 | −0.534 | 8.17 | −18.95 |
| ATOM | 340 | OE2 | GLU | 346 | −5.950 | −1.513 | −23.517 | 0.00 | 0.00 | −0.534 | 8.17 | −18.95 |
| ATOM | 341 | N | LEU | 347 | −9.973 | −4.165 | −19.496 | 0.00 | 0.00 | −0.650 | 9.00 | −17.40 |
| ATOM | 342 | HN | LEU | 347 | −10.122 | −3.299 | −18.959 | 0.00 | 0.00 | 0.439 | 0.00 | 0.00 |
| ATOM | 343 | CA | LEU | 347 | −10.028 | −5.452 | −18.910 | 0.00 | 0.00 | 0.158 | 9.40 | 4.00 |
| ATOM | 344 | HA | LEU | 347 | −9.121 | −6.001 | −19.164 | 0.00 | 0.00 | 0.053 | 0.00 | 0.00 |
| ATOM | 345 | C | LEU | 347 | −11.225 | −6.183 | −19.436 | 0.00 | 0.00 | 0.396 | 9.82 | 4.00 |
| ATOM | 346 | O | LEU | 347 | −11.059 | −7.053 | −20.281 | 0.00 | 0.00 | −0.396 | 8.17 | −17.40 |
| ATOM | 347 | CB | LEU | 347 | −9.967 | −5.426 | −17.377 | 0.00 | 0.00 | −0.106 | 12.77 | 4.00 |
| ATOM | 348 | HB1 | LEU | 347 | −10.938 | −5.206 | −16.934 | 0.00 | 0.00 | 0.053 | 0.00 | 0.00 |
| ATOM | 349 | HB2 | LEU | 347 | −9.273 | −4.670 | −17.009 | 0.00 | 0.00 | 0.053 | 0.00 | 0.00 |
| ATOM | 350 | CG | LEU | 347 | −9.507 | −6.773 | −16.797 | 0.00 | 0.00 | −0.053 | 9.40 | 4.00 |
| ATOM | 351 | HG | LEU | 347 | −8.652 | −7.163 | −17.349 | 0.00 | 0.00 | 0.053 | 0.00 | 0.00 |
| ATOM | 352 | CD1 | LEU | 347 | −9.094 | −6.624 | −15.327 | 0.00 | 0.00 | −0.159 | 16.15 | 4.00 |
| ATOM | 353 | HD11 | LEU | 347 | −8.773 | −7.591 | −14.940 | 0.00 | 0.00 | 0.053 | 0.00 | 0.00 |
| ATOM | 354 | HD12 | LEU | 347 | −8.273 | −5.911 | −15.250 | 0.00 | 0.00 | 0.053 | 0.00 | 0.00 |
| ATOM | 355 | HD13 | LEU | 347 | −9.943 | −6.264 | −14.745 | 0.00 | 0.00 | 0.053 | 0.00 | 0.00 |
| ATOM | 356 | CD2 | LEU | 347 | −10.545 | −7.881 | −17.030 | 0.00 | 0.00 | −0.159 | 16.15 | 4.00 |
| ATOM | 357 | HD21 | LEU | 347 | −10.181 | −8.816 | −16.605 | 0.00 | 0.00 | 0.053 | 0.00 | 0.00 |
| ATOM | 358 | HD22 | LEU | 347 | −11.484 | −7.606 | −16.551 | 0.00 | 0.00 | 0.053 | 0.00 | 0.00 |
| ATOM | 359 | HD23 | LEU | 347 | −10.708 | −8.008 | −18.100 | 0.00 | 0.00 | 0.053 | 0.00 | 0.00 |
| ATOM | 360 | N | ILE | 348 | −12.469 | −5.774 | −19.107 | 0.00 | 0.00 | −0.650 | 9.00 | −17.40 |
| ATOM | 361 | HN | ILE | 348 | −12.616 | −4.940 | −18.521 | 0.00 | 0.00 | 0.439 | 0.00 | 0.00 |
| ATOM | 362 | CA | ILE | 348 | −13.586 | −6.541 | −19.601 | 0.00 | 0.00 | 0.158 | 9.40 | 4.00 |
| ATOM | 363 | HA | ILE | 348 | −13.183 | −7.481 | −19.979 | 0.00 | 0.00 | 0.053 | 0.00 | 0.00 |
| ATOM | 364 | C | ILE | 348 | −14.220 | −5.737 | −20.677 | 0.00 | 0.00 | 0.396 | 9.82 | 4.00 |
| ATOM | 365 | O | ILE | 348 | −13.722 | −4.673 | −21.025 | 0.00 | 0.00 | −0.396 | 8.17 | −17.40 |
| ATOM | 366 | CB | ILE | 348 | −14.687 | −6.841 | −18.614 | 0.00 | 0.00 | −0.053 | 9.40 | 4.00 |
| ATOM | 367 | HB | ILE | 348 | −14.243 | −7.079 | −17.647 | 0.00 | 0.00 | 0.053 | 0.00 | 0.00 |
| ATOM | 368 | CG1 | ILE | 348 | −15.476 | −8.078 | −19.082 | 0.00 | 0.00 | −0.106 | 12.77 | 4.00 |
| ATOM | 369 | HG11 | ILE | 348 | −14.843 | −8.923 | −19.354 | 0.00 | 0.00 | 0.053 | 0.00 | 0.00 |
| ATOM | 370 | HG12 | ILE | 348 | −16.095 | −7.895 | −19.960 | 0.00 | 0.00 | 0.053 | 0.00 | 0.00 |
| ATOM | 371 | CG2 | ILE | 348 | −15.599 | −5.601 | −18.483 | 0.00 | 0.00 | −0.159 | 16.15 | 4.00 |
| ATOM | 372 | HG21 | ILE | 348 | −16.397 | −5.810 | −17.771 | 0.00 | 0.00 | 0.053 | 0.00 | 0.00 |
| ATOM | 373 | HG22 | ILE | 348 | −15.012 | −4.753 | −18.131 | 0.00 | 0.00 | 0.053 | 0.00 | 0.00 |
| ATOM | 374 | HG23 | ILE | 348 | −16.032 | −5.364 | −19.455 | 0.00 | 0.00 | 0.053 | 0.00 | 0.00 |
| ATOM | 375 | CD1 | ILE | 348 | −16.435 | −8.626 | −18.029 | 0.00 | 0.00 | −0.159 | 16.15 | 4.00 |
| ATOM | 376 | HD11 | ILE | 348 | −16.956 | −9.496 | −18.429 | 0.00 | 0.00 | 0.053 | 0.00 | 0.00 |
| ATOM | 377 | HD12 | ILE | 348 | −15.873 | −8.916 | −17.141 | 0.00 | 0.00 | 0.053 | 0.00 | 0.00 |
| ATOM | 378 | HD13 | ILE | 348 | −17.162 | −7.858 | −17.764 | 0.00 | 0.00 | 0.053 | 0.00 | 0.00 |
| ATOM | 379 | N | TYR | 349 | −15.355 | −6.227 | −21.211 | 0.00 | 0.00 | −0.650 | 9.00 | −17.40 |
| ATOM | 380 | HN | TYR | 349 | −15.747 | −7.091 | −20.809 | 0.00 | 0.00 | 0.439 | 0.00 | 0.00 |
| ATOM | 381 | CA | TYR | 349 | −16.048 | −5.618 | −22.303 | 0.00 | 0.00 | 0.158 | 9.40 | 4.00 |
| ATOM | 382 | HA | TYR | 349 | −15.476 | −5.766 | −23.219 | 0.00 | 0.00 | 0.053 | 0.00 | 0.00 |
| ATOM | 383 | C | TYR | 349 | −16.201 | −4.149 | −22.034 | 0.00 | 0.00 | 0.396 | 9.82 | 4.00 |
| ATOM | 384 | O | TYR | 349 | −16.089 | −3.342 | −22.956 | 0.00 | 0.00 | −0.396 | 8.17 | −17.40 |
| ATOM | 385 | CB | TYR | 349 | −17.451 | −6.220 | −22.490 | 0.00 | 0.00 | −0.106 | 12.77 | 4.00 |
| ATOM | 386 | HB1 | TYR | 349 | −18.078 | −5.907 | −21.655 | 0.00 | 0.00 | 0.053 | 0.00 | 0.00 |
| ATOM | 387 | HB2 | TYR | 349 | −17.364 | −7.306 | −22.511 | 0.00 | 0.00 | 0.053 | 0.00 | 0.00 |
| ATOM | 388 | CG | TYR | 349 | −18.015 | −5.722 | −23.775 | 0.00 | 0.00 | 0.000 | 7.26 | 0.60 |
| ATOM | 389 | CD1 | TYR | 349 | −17.694 | −6.354 | −24.954 | 0.00 | 0.00 | −0.127 | 10.80 | 0.60 |
| ATOM | 390 | HD1 | TYR | 349 | −17.025 | −7.214 | −24.939 | 0.00 | 0.00 | 0.127 | 0.00 | 0.00 |
| ATOM | 391 | CD2 | TYR | 349 | −18.864 | −4.641 | −23.807 | 0.00 | 0.00 | −0.127 | 10.80 | 0.60 |
| ATOM | 392 | HD2 | TYR | 349 | −19.130 | −4.134 | −22.880 | 0.00 | 0.00 | 0.127 | 0.00 | 0.00 |
| ATOM | 393 | CE1 | TYR | 349 | −18.207 | −5.913 | −26.150 | 0.00 | 0.00 | −0.127 | 10.80 | 0.60 |
| ATOM | 394 | HE1 | TYR | 349 | −17.944 | −6.421 | −27.078 | 0.00 | 0.00 | 0.127 | 0.00 | 0.00 |
| ATOM | 395 | CE2 | TYR | 349 | −19.380 | −4.195 | −25.002 | 0.00 | 0.00 | −0.127 | 10.80 | 0.60 |
| ATOM | 396 | HE2 | TYR | 349 | −20.050 | −3.336 | −25.018 | 0.00 | 0.00 | 0.127 | 0.00 | 0.00 |
| ATOM | 397 | CZ | TYR | 349 | −19.052 | −4.831 | −26.175 | 0.00 | 0.00 | 0.027 | 7.26 | 0.60 |
| ATOM | 398 | OH | TYR | 349 | −19.580 | −4.376 | −27.402 | 0.00 | 0.00 | −0.451 | 10.94 | −17.40 |
| ATOM | 399 | HH | TYR | 349 | −19.396 | −3.368 | −27.503 | 0.00 | 0.00 | 0.424 | 0.00 | 0.00 |
| ATOM | 400 | N | ILE | 350 | −16.440 | −3.747 | −20.769 | 0.00 | 0.00 | −0.650 | 9.00 | −17.40 |
| ATOM | 401 | HN | ILE | 350 | −16.492 | −4.420 | −19.991 | 0.00 | 0.00 | 0.439 | 0.00 | 0.00 |
| ATOM | 402 | CA | ILE | 350 | −16.618 | −2.339 | −20.556 | 0.00 | 0.00 | 0.158 | 9.40 | 4.00 |
| ATOM | 403 | HA | ILE | 350 | −16.237 | −1.784 | −21.414 | 0.00 | 0.00 | 0.053 | 0.00 | 0.00 |
| ATOM | 404 | C | ILE | 350 | −15.871 | −1.911 | −19.321 | 0.00 | 0.00 | 0.396 | 9.82 | 4.00 |
| ATOM | 405 | O | ILE | 350 | −15.575 | −2.727 | −18.450 | 0.00 | 0.00 | −0.396 | 8.17 | −17.40 |
| ATOM | 406 | CB | ILE | 350 | −18.071 | −1.983 | −20.370 | 0.00 | 0.00 | −0.053 | 9.40 | 4.00 |
| ATOM | 407 | HB | ILE | 350 | −18.474 | −2.531 | −19.518 | 0.00 | 0.00 | 0.053 | 0.00 | 0.00 |
| ATOM | 408 | CG1 | ILE | 350 | −18.883 | −2.445 | −21.592 | 0.00 | 0.00 | −0.106 | 12.77 | 4.00 |
| ATOM | 409 | HG11 | ILE | 350 | −18.674 | −3.474 | −21.886 | 0.00 | 0.00 | 0.053 | 0.00 | 0.00 |
| ATOM | 410 | HG12 | ILE | 350 | −18.699 | −1.847 | −22.485 | 0.00 | 0.00 | 0.053 | 0.00 | 0.00 |
| ATOM | 411 | CG2 | ILE | 350 | −18.201 | −0.473 | −20.115 | 0.00 | 0.00 | −0.159 | 16.15 | 4.00 |
| ATOM | 412 | HG21 | ILE | 350 | −19.252 | −0.216 | −19.981 | 0.00 | 0.00 | 0.053 | 0.00 | 0.00 |
| ATOM | 413 | HG22 | ILE | 350 | −17.644 | −0.206 | −19.217 | 0.00 | 0.00 | 0.053 | 0.00 | 0.00 |
| ATOM | 414 | HG23 | ILE | 350 | −17.799 | 0.076 | −20.967 | 0.00 | 0.00 | 0.053 | 0.00 | 0.00 |
| ATOM | 415 | CD1 | ILE | 350 | −20.394 | −2.387 | −21.373 | 0.00 | 0.00 | −0.159 | 16.15 | 4.00 |
| ATOM | 416 | HD11 | ILE | 350 | −20.905 | −2.726 | −22.274 | 0.00 | 0.00 | 0.053 | 0.00 | 0.00 |
| ATOM | 417 | HD12 | ILE | 350 | −20.665 | −3.032 | −20.537 | 0.00 | 0.00 | 0.053 | 0.00 | 0.00 |
| ATOM | 418 | HD13 | ILE | 350 | −20.690 | −1.362 | −21.152 | 0.00 | 0.00 | 0.053 | 0.00 | 0.00 |
| ATOM | 419 | N | ASP | 351 | −15.515 | −0.603 | −19.282 | 0.00 | 0.00 | −0.650 | 9.00 | −17.40 |
| ATOM | 420 | HN | ASP | 351 | −15.672 | −0.075 | −20.152 | 0.00 | 0.00 | 0.439 | 0.00 | 0.00 |
| ATOM | 421 | CA | ASP | 351 | −14.950 | 0.149 | −18.199 | 0.00 | 0.00 | 0.158 | 9.40 | 4.00 |
| ATOM | 422 | HA | ASP | 351 | −15.725 | 0.800 | −17.793 | 0.00 | 0.00 | 0.053 | 0.00 | 0.00 |
| ATOM | 423 | C | ASP | 351 | −13.779 | 1.019 | −18.592 | 0.00 | 0.00 | 0.396 | 9.82 | 4.00 |
| ATOM | 424 | O | ASP | 351 | −13.505 | 1.215 | −19.774 | 0.00 | 0.00 | −0.396 | 8.17 | −17.40 |
| ATOM | 425 | CB | ASP | 351 | −14.807 | −0.619 | −16.892 | 0.00 | 0.00 | −0.336 | 12.77 | 4.00 |
| ATOM | 426 | HB1 | ASP | 351 | −14.150 | −0.006 | −16.276 | 0.00 | 0.00 | 0.053 | 0.00 | 0.00 |
| ATOM | 427 | HB2 | ASP | 351 | −14.371 | −1.577 | −17.175 | 0.00 | 0.00 | 0.053 | 0.00 | 0.00 |
| ATOM | 428 | CG | ASP | 351 | −16.222 | −0.732 | −16.346 | 0.00 | 0.00 | 0.297 | 9.82 | 4.00 |
| ATOM | 429 | OD1 | ASP | 351 | −16.748 | 0.308 | −15.863 | 0.00 | 0.00 | −0.534 | 8.17 | −18.95 |
| ATOM | 430 | OD2 | ASP | 351 | −16.807 | −1.845 | −16.412 | 0.00 | 0.00 | −0.534 | 8.17 | −18.95 |
| ATOM | 431 | N | ILE | 352 | −13.068 | 1.584 | −17.589 | 0.00 | 0.00 | −0.650 | 9.00 | −17.40 |
| ATOM | 432 | HN | ILE | 352 | −13.182 | 1.214 | −16.634 | 0.00 | 0.00 | 0.439 | 0.00 | 0.00 |
| ATOM | 433 | CA | ILE | 352 | −12.155 | 2.685 | −17.801 | 0.00 | 0.00 | 0.158 | 9.40 | 4.00 |
| ATOM | 434 | HA | ILE | 352 | −12.709 | 3.454 | −18.339 | 0.00 | 0.00 | 0.053 | 0.00 | 0.00 |
| ATOM | 435 | C | ILE | 352 | −10.879 | 2.482 | −18.588 | 0.00 | 0.00 | 0.396 | 9.82 | 4.00 |
| ATOM | 436 | O | ILE | 352 | −10.619 | 3.275 | −19.487 | 0.00 | 0.00 | −0.396 | 8.17 | −17.40 |
| ATOM | 437 | CB | ILE | 352 | −11.803 | 3.419 | −16.535 | 0.00 | 0.00 | −0.053 | 9.40 | 4.00 |
| ATOM | 438 | HB | ILE | 352 | −12.717 | 3.653 | −15.989 | 0.00 | 0.00 | 0.053 | 0.00 | 0.00 |
| ATOM | 439 | CG1 | ILE | 352 | −11.175 | 4.779 | −16.884 | 0.00 | 0.00 | −0.106 | 12.77 | 4.00 |
| ATOM | 440 | HG11 | ILE | 352 | −10.324 | 4.699 | −17.560 | 0.00 | 0.00 | 0.053 | 0.00 | 0.00 |
| ATOM | 441 | HG12 | ILE | 352 | −10.803 | 5.317 | −16.012 | 0.00 | 0.00 | 0.053 | 0.00 | 0.00 |
| ATOM | 442 | CG2 | ILE | 352 | −10.898 | 2.532 | −15.672 | 0.00 | 0.00 | −0.159 | 16.15 | 4.00 |
| ATOM | 443 | HG21 | ILE | 352 | −10.640 | 3.060 | −14.754 | 0.00 | 0.00 | 0.053 | 0.00 | 0.00 |
| ATOM | 444 | HG22 | ILE | 352 | −11.422 | 1.609 | −15.425 | 0.00 | 0.00 | 0.053 | 0.00 | 0.00 |
| ATOM | 445 | HG23 | ILE | 352 | −9.987 | 2.296 | −16.223 | 0.00 | 0.00 | 0.053 | 0.00 | 0.00 |
| ATOM | 446 | CD1 | ILE | 352 | −12.150 | 5.735 | −17.568 | 0.00 | 0.00 | −0.159 | 16.15 | 4.00 |
| ATOM | 447 | HD11 | ILE | 352 | −11.644 | 6.675 | −17.787 | 0.00 | 0.00 | 0.053 | 0.00 | 0.00 |
| ATOM | 448 | HD12 | ILE | 352 | −12.505 | 5.289 | −18.497 | 0.00 | 0.00 | 0.053 | 0.00 | 0.00 |
| ATOM | 449 | HD13 | ILE | 352 | −12.997 | 5.924 | −16.909 | 0.00 | 0.00 | 0.053 | 0.00 | 0.00 |
| ATOM | 450 | N | VAL | 353 | −10.041 | 1.459 | −18.324 | 0.00 | 0.00 | −0.650 | 9.00 | −17.40 |
| ATOM | 451 | HN | VAL | 353 | −10.310 | 0.715 | −17.665 | 0.00 | 0.00 | 0.439 | 0.00 | 0.00 |
| ATOM | 452 | CA | VAL | 353 | −8.755 | 1.431 | −18.988 | 0.00 | 0.00 | 0.158 | 9.40 | 4.00 |
| ATOM | 453 | HA | VAL | 353 | −8.189 | 2.335 | −18.762 | 0.00 | 0.00 | 0.053 | 0.00 | 0.00 |
| ATOM | 454 | C | VAL | 353 | −8.932 | 1.343 | −20.466 | 0.00 | 0.00 | 0.396 | 9.82 | 4.00 |
| ATOM | 455 | O | VAL | 353 | −8.302 | 2.088 | −21.216 | 0.00 | 0.00 | −0.396 | 8.17 | −17.40 |
| ATOM | 456 | CB | VAL | 353 | −7.857 | 0.307 | −18.555 | 0.00 | 0.00 | −0.053 | 9.40 | 4.00 |
| ATOM | 457 | HB | VAL | 353 | −8.399 | −0.630 | −18.681 | 0.00 | 0.00 | 0.053 | 0.00 | 0.00 |
| ATOM | 458 | CG1 | VAL | 353 | −6.599 | 0.330 | −19.438 | 0.00 | 0.00 | −0.159 | 16.15 | 4.00 |
| ATOM | 459 | HG11 | VAL | 353 | −5.931 | −0.479 | −19.141 | 0.00 | 0.00 | 0.053 | 0.00 | 0.00 |
| ATOM | 460 | HG12 | VAL | 353 | −6.885 | 0.200 | −20.482 | 0.00 | 0.00 | 0.053 | 0.00 | 0.00 |
| ATOM | 461 | HG13 | VAL | 353 | −6.088 | 1.285 | −19.318 | 0.00 | 0.00 | 0.053 | 0.00 | 0.00 |
| ATOM | 462 | CG2 | VAL | 353 | −7.523 | 0.491 | −17.065 | 0.00 | 0.00 | −0.159 | 16.15 | 4.00 |
| ATOM | 463 | HG21 | VAL | 353 | −6.871 | −0.318 | −16.736 | 0.00 | 0.00 | 0.053 | 0.00 | 0.00 |
| ATOM | 464 | HG22 | VAL | 353 | −7.017 | 1.446 | −16.921 | 0.00 | 0.00 | 0.053 | 0.00 | 0.00 |
| ATOM | 465 | HG23 | VAL | 353 | −8.443 | 0.476 | −16.481 | 0.00 | 0.00 | 0.053 | 0.00 | 0.00 |
| ATOM | 466 | N | GLY | 354 | −9.791 | 0.424 | −20.934 | 0.00 | 0.00 | −0.650 | 9.00 | −17.40 |
| ATOM | 467 | HN | GLY | 354 | −10.300 | −0.188 | −20.281 | 0.00 | 0.00 | 0.439 | 0.00 | 0.00 |
| ATOM | 468 | CA | GLY | 354 | −9.996 | 0.299 | −22.345 | 0.00 | 0.00 | 0.105 | 9.40 | 4.00 |
| ATOM | 469 | HA1 | GLY | 354 | −10.574 | −0.600 | −22.559 | 0.00 | 0.00 | 0.053 | 0.00 | 0.00 |
| ATOM | 470 | HA2 | GLY | 354 | −9.036 | 0.232 | −22.856 | 0.00 | 0.00 | 0.053 | 0.00 | 0.00 |
| ATOM | 471 | C | GLY | 354 | −10.739 | 1.489 | −22.860 | 0.00 | 0.00 | 0.396 | 9.82 | 4.00 |
| ATOM | 472 | O | GLY | 354 | −10.519 | 1.927 | −23.987 | 0.00 | 0.00 | −0.396 | 8.17 | −17.40 |
| ATOM | 473 | N | LEU | 355 | −11.675 | 2.029 | −22.061 | 0.00 | 0.00 | −0.650 | 9.00 | −17.40 |
| ATOM | 474 | HN | LEU | 355 | −11.838 | 1.644 | −21.120 | 0.00 | 0.00 | 0.439 | 0.00 | 0.00 |
| ATOM | 475 | CA | LEU | 355 | −12.446 | 3.145 | −22.528 | 0.00 | 0.00 | 0.158 | 9.40 | 4.00 |
| ATOM | 476 | HA | LEU | 355 | −12.932 | 2.839 | −23.454 | 0.00 | 0.00 | 0.053 | 0.00 | 0.00 |
| ATOM | 477 | C | LEU | 355 | −11.496 | 4.279 | −22.755 | 0.00 | 0.00 | 0.396 | 9.82 | 4.00 |
| ATOM | 478 | O | LEU | 355 | −11.586 | 4.988 | −23.757 | 0.00 | 0.00 | −0.396 | 8.17 | −17.40 |
| ATOM | 479 | CB | LEU | 355 | −13.517 | 3.573 | −21.495 | 0.00 | 0.00 | −0.106 | 12.77 | 4.00 |
| ATOM | 480 | HB1 | LEU | 355 | −12.993 | 3.968 | −20.625 | 0.00 | 0.00 | 0.053 | 0.00 | 0.00 |
| ATOM | 481 | HB2 | LEU | 355 | −14.099 | 2.687 | −21.240 | 0.00 | 0.00 | 0.053 | 0.00 | 0.00 |
| ATOM | 482 | CG | LEU | 355 | −14.536 | 4.659 | −21.926 | 0.00 | 0.00 | −0.053 | 9.40 | 4.00 |
| ATOM | 483 | HG | LEU | 355 | −15.099 | 4.322 | −22.796 | 0.00 | 0.00 | 0.053 | 0.00 | 0.00 |
| ATOM | 484 | CD1 | LEU | 355 | −15.521 | 4.946 | −20.780 | 0.00 | 0.00 | −0.159 | 16.15 | 4.00 |
| ATOM | 485 | HD11 | LEU | 355 | −16.232 | 5.710 | −21.094 | 0.00 | 0.00 | 0.053 | 0.00 | 0.00 |
| ATOM | 486 | HD12 | LEU | 355 | −16.059 | 4.033 | −20.525 | 0.00 | 0.00 | 0.053 | 0.00 | 0.00 |
| ATOM | 487 | HD13 | LEU | 355 | −14.971 | 5.299 | −19.907 | 0.00 | 0.00 | 0.053 | 0.00 | 0.00 |
| ATOM | 488 | CD2 | LEU | 355 | −13.883 | 5.954 | −22.434 | 0.00 | 0.00 | −0.159 | 16.15 | 4.00 |
| ATOM | 489 | HD21 | LEU | 355 | −14.658 | 6.666 | −22.717 | 0.00 | 0.00 | 0.053 | 0.00 | 0.00 |
| ATOM | 490 | HD22 | LEU | 355 | −13.266 | 6.384 | −21.645 | 0.00 | 0.00 | 0.053 | 0.00 | 0.00 |
| ATOM | 491 | HD23 | LEU | 355 | −13.261 | 5.732 | −23.301 | 0.00 | 0.00 | 0.053 | 0.00 | 0.00 |
| ATOM | 492 | N | ALA | 356 | −10.560 | 4.468 | −21.809 | 0.00 | 0.00 | −0.650 | 9.00 | −17.40 |
| ATOM | 493 | HN | ALA | 356 | −10.533 | 3.806 | −21.021 | 0.00 | 0.00 | 0.439 | 0.00 | 0.00 |
| ATOM | 494 | CA | ALA | 356 | −9.595 | 5.532 | −21.824 | 0.00 | 0.00 | 0.158 | 9.40 | 4.00 |
| ATOM | 495 | HA | ALA | 356 | −10.076 | 6.499 | −21.973 | 0.00 | 0.00 | 0.053 | 0.00 | 0.00 |
| ATOM | 496 | C | ALA | 356 | −8.599 | 5.345 | −22.926 | 0.00 | 0.00 | 0.396 | 9.82 | 4.00 |
| ATOM | 497 | O | ALA | 356 | −8.168 | 6.333 | −23.519 | 0.00 | 0.00 | −0.396 | 8.17 | −17.40 |
| ATOM | 498 | CB | ALA | 356 | −8.797 | 5.627 | −20.511 | 0.00 | 0.00 | −0.159 | 16.15 | 4.00 |
| ATOM | 499 | HB1 | ALA | 356 | −8.083 | 6.448 | −20.578 | 0.00 | 0.00 | 0.053 | 0.00 | 0.00 |
| ATOM | 500 | HB2 | ALA | 356 | −9.481 | 5.808 | −19.682 | 0.00 | 0.00 | 0.053 | 0.00 | 0.00 |
| ATOM | 501 | HB3 | ALA | 356 | −8.262 | 4.693 | −20.342 | 0.00 | 0.00 | 0.053 | 0.00 | 0.00 |
| ATOM | 502 | N | GLN | 357 | −8.210 | 4.078 | −23.202 | 0.00 | 0.00 | −0.650 | 9.00 | −17.40 |
| ATOM | 503 | HN | GLN | 357 | −8.669 | 3.319 | −22.679 | 0.00 | 0.00 | 0.439 | 0.00 | 0.00 |
| ATOM | 504 | CA | GLN | 357 | −7.205 | 3.702 | −24.168 | 0.00 | 0.00 | 0.158 | 9.40 | 4.00 |
| ATOM | 505 | HA | GLN | 357 | −6.204 | 3.819 | −23.752 | 0.00 | 0.00 | 0.053 | 0.00 | 0.00 |
| ATOM | 506 | C | GLN | 357 | −7.300 | 4.553 | −25.392 | 0.00 | 0.00 | 0.396 | 9.82 | 4.00 |
| ATOM | 507 | O | GLN | 357 | −8.265 | 4.470 | −26.149 | 0.00 | 0.00 | −0.396 | 8.17 | −17.40 |
| ATOM | 508 | CB | GLN | 357 | −7.377 | 2.248 | −24.644 | 0.00 | 0.00 | −0.106 | 12.77 | 4.00 |
| ATOM | 509 | HB1 | GLN | 357 | −8.386 | 2.135 | −25.040 | 0.00 | 0.00 | 0.053 | 0.00 | 0.00 |
| ATOM | 510 | HB2 | GLN | 357 | −7.224 | 1.588 | −23.790 | 0.00 | 0.00 | 0.053 | 0.00 | 0.00 |
| ATOM | 511 | CG | GLN | 357 | −6.400 | 1.824 | −25.738 | 0.00 | 0.00 | −0.106 | 12.77 | 4.00 |
| ATOM | 512 | HG1 | GLN | 357 | −6.112 | 2.711 | −26.302 | 0.00 | 0.00 | 0.053 | 0.00 | 0.00 |
| ATOM | 513 | HG2 | GLN | 357 | −6.902 | 1.104 | −26.385 | 0.00 | 0.00 | 0.053 | 0.00 | 0.00 |
| ATOM | 514 | CD | GLN | 357 | −5.188 | 1.194 | −25.076 | 0.00 | 0.00 | 0.396 | 9.82 | 4.00 |
| ATOM | 515 | OE1 | GLN | 357 | −4.308 | 0.685 | −25.766 | 0.00 | 0.00 | −0.396 | 8.17 | −17.40 |
| ATOM | 516 | NE2 | GLN | 357 | −5.142 | 1.209 | −23.718 | 0.00 | 0.00 | −0.879 | 13.25 | −17.40 |
| ATOM | 517 | HE21 | GLN | 357 | −5.904 | 1.648 | −23.182 | 0.00 | 0.00 | 0.439 | 0.00 | 0.00 |
| ATOM | 518 | HE22 | GLN | 357 | −4.345 | 0.781 | −23.225 | 0.00 | 0.00 | 0.439 | 0.00 | 0.00 |
| ATOM | 519 | N | PHE | 358 | −6.296 | 5.429 | −25.599 | 0.00 | 0.00 | −0.650 | 9.00 | −17.40 |
| ATOM | 520 | HN | PHE | 358 | −5.523 | 5.504 | −24.922 | 0.00 | 0.00 | 0.439 | 0.00 | 0.00 |
| ATOM | 521 | CA | PHE | 358 | −6.315 | 6.254 | −26.768 | 0.00 | 0.00 | 0.158 | 9.40 | 4.00 |
| ATOM | 522 | HA | PHE | 358 | −7.300 | 6.717 | −26.825 | 0.00 | 0.00 | 0.053 | 0.00 | 0.00 |
| ATOM | 523 | C | PHE | 358 | −6.058 | 5.368 | −27.946 | 0.00 | 0.00 | 0.396 | 9.82 | 4.00 |
| ATOM | 524 | O | PHE | 358 | −6.739 | 5.465 | −28.966 | 0.00 | 0.00 | −0.396 | 8.17 | −17.40 |
| ATOM | 525 | CB | PHE | 358 | −5.261 | 7.369 | −26.758 | 0.00 | 0.00 | −0.106 | 12.77 | 4.00 |
| ATOM | 526 | HB1 | PHE | 358 | −4.302 | 6.883 | −26.937 | 0.00 | 0.00 | 0.053 | 0.00 | 0.00 |
| ATOM | 527 | HB2 | PHE | 358 | −5.317 | 7.832 | −25.773 | 0.00 | 0.00 | 0.053 | 0.00 | 0.00 |
| ATOM | 528 | CG | PHE | 358 | −5.637 | 8.307 | −27.851 | 0.00 | 0.00 | 0.000 | 7.26 | 0.60 |
| ATOM | 529 | CD1 | PHE | 358 | −6.539 | 9.317 | −27.607 | 0.00 | 0.00 | −0.127 | 10.80 | 0.60 |
| ATOM | 530 | HD1 | PHE | 358 | −6.971 | 9.425 | −26.612 | 0.00 | 0.00 | 0.127 | 0.00 | 0.00 |
| ATOM | 531 | CD2 | PHE | 358 | −5.106 | 8.179 | −29.113 | 0.00 | 0.00 | −0.127 | 10.80 | 0.60 |
| ATOM | 532 | HD2 | PHE | 358 | −4.395 | 7.380 | −29.322 | 0.00 | 0.00 | 0.127 | 0.00 | 0.00 |
| ATOM | 533 | CE1 | PHE | 358 | −6.902 | 10.192 | −28.603 | 0.00 | 0.00 | −0.127 | 10.80 | 0.60 |
| ATOM | 534 | HE1 | PHE | 358 | −7.615 | 10.990 | −28.396 | 0.00 | 0.00 | 0.127 | 0.00 | 0.00 |
| ATOM | 535 | CE2 | PHE | 358 | −5.465 | 9.051 | −30.113 | 0.00 | 0.00 | −0.127 | 10.80 | 0.60 |
| ATOM | 536 | HE2 | PHE | 358 | −5.035 | 8.943 | −31.109 | 0.00 | 0.00 | 0.127 | 0.00 | 0.00 |
| ATOM | 537 | CZ | PHE | 358 | −6.365 | 10.059 | −29.860 | 0.00 | 0.00 | −0.127 | 10.80 | 0.60 |
| ATOM | 538 | HZ | PHE | 358 | −6.651 | 10.749 | −30.653 | 0.00 | 0.00 | 0.127 | 0.00 | 0.00 |
| ATOM | 539 | N | LYS | 359 | −5.053 | 4.470 | −27.841 | 0.00 | 0.00 | −0.650 | 9.00 | −17.40 |
| ATOM | 540 | HN | LYS | 359 | −4.455 | 4.435 | −27.003 | 0.00 | 0.00 | 0.439 | 0.00 | 0.00 |
| ATOM | 541 | CA | LYS | 359 | −4.848 | 3.570 | −28.935 | 0.00 | 0.00 | 0.158 | 9.40 | 4.00 |
| ATOM | 542 | HA | LYS | 359 | −5.794 | 3.349 | −29.428 | 0.00 | 0.00 | 0.053 | 0.00 | 0.00 |
| ATOM | 543 | C | LYS | 359 | −4.256 | 2.290 | −28.452 | 0.00 | 0.00 | 0.396 | 9.82 | 4.00 |
| ATOM | 544 | O | LYS | 359 | −3.419 | 2.254 | −27.550 | 0.00 | 0.00 | −0.396 | 8.17 | −17.40 |
| ATOM | 545 | CB | LYS | 359 | −3.966 | 4.089 | −30.076 | 0.00 | 0.00 | −0.106 | 12.77 | 4.00 |
| ATOM | 546 | HB1 | LYS | 359 | −2.926 | 4.156 | −29.757 | 0.00 | 0.00 | 0.053 | 0.00 | 0.00 |
| ATOM | 547 | HB2 | LYS | 359 | −4.292 | 5.080 | −30.393 | 0.00 | 0.00 | 0.053 | 0.00 | 0.00 |
| ATOM | 548 | CG | LYS | 359 | −4.039 | 3.150 | −31.284 | 0.00 | 0.00 | −0.106 | 12.77 | 4.00 |
| ATOM | 549 | HG1 | LYS | 359 | −3.745 | 2.129 | −31.041 | 0.00 | 0.00 | 0.053 | 0.00 | 0.00 |
| ATOM | 550 | HG2 | LYS | 359 | −3.390 | 3.465 | −32.101 | 0.00 | 0.00 | 0.053 | 0.00 | 0.00 |
| ATOM | 551 | CD | LYS | 359 | −5.449 | 3.056 | −31.875 | 0.00 | 0.00 | −0.106 | 12.77 | 4.00 |
| ATOM | 552 | HD1 | LYS | 359 | −5.750 | 4.050 | −32.204 | 0.00 | 0.00 | 0.053 | 0.00 | 0.00 |
| ATOM | 553 | HD2 | LYS | 359 | −6.122 | 2.689 | −31.100 | 0.00 | 0.00 | 0.053 | 0.00 | 0.00 |
| ATOM | 554 | CE | LYS | 359 | −5.560 | 2.114 | −33.074 | 0.00 | 0.00 | 0.099 | 12.77 | 4.00 |
| ATOM | 555 | HE1 | LYS | 359 | −6.599 | 2.049 | −33.396 | 0.00 | 0.00 | 0.053 | 0.00 | 0.00 |
| ATOM | 556 | HE2 | LYS | 359 | −5.209 | 1.121 | −32.795 | 0.00 | 0.00 | 0.053 | 0.00 | 0.00 |
| ATOM | 557 | NZ | LYS | 359 | −4.736 | 2.626 | −34.189 | 0.00 | 0.00 | −0.045 | 13.25 | −39.20 |
| ATOM | 558 | HZ1 | LYS | 359 | −4.813 | 1.989 | −34.995 | 0.00 | 0.00 | 0.280 | 0.00 | 0.00 |
| ATOM | 559 | HZ2 | LYS | 359 | −5.068 | 3.563 | −34.460 | 0.00 | 0.00 | 0.280 | 0.00 | 0.00 |
| ATOM | 560 | HZ3 | LYS | 359 | −3.752 | 2.684 | −33.891 | 0.00 | 0.00 | 0.280 | 0.00 | 0.00 |
| ATOM | 561 | N | LYS | 360 | −4.702 | 1.199 | −29.098 | 0.00 | 0.00 | −0.650 | 9.00 | −17.40 |
| ATOM | 562 | HN | LYS | 360 | −5.381 | 1.350 | −29.857 | 0.00 | 0.00 | 0.439 | 0.00 | 0.00 |
| ATOM | 563 | CA | LYS | 360 | −4.319 | −0.152 | −28.827 | 0.00 | 0.00 | 0.158 | 9.40 | 4.00 |
| ATOM | 564 | HA | LYS | 360 | −4.521 | −0.363 | −27.777 | 0.00 | 0.00 | 0.053 | 0.00 | 0.00 |
| ATOM | 565 | C | LYS | 360 | −2.859 | −0.297 | −29.120 | 0.00 | 0.00 | 0.396 | 9.82 | 4.00 |
| ATOM | 566 | O | LYS | 360 | −2.127 | −0.953 | −28.381 | 0.00 | 0.00 | −0.396 | 8.17 | −17.40 |
| ATOM | 567 | CB | LYS | 360 | −5.079 | −1.117 | −29.755 | 0.00 | 0.00 | −0.106 | 12.77 | 4.00 |
| ATOM | 568 | HB1 | LYS | 360 | −4.827 | −0.860 | −30.784 | 0.00 | 0.00 | 0.053 | 0.00 | 0.00 |
| ATOM | 569 | HB2 | LYS | 360 | −6.145 | −0.988 | −29.570 | 0.00 | 0.00 | 0.053 | 0.00 | 0.00 |
| ATOM | 570 | CG | LYS | 360 | −4.770 | −2.603 | −29.577 | 0.00 | 0.00 | −0.106 | 12.77 | 4.00 |
| ATOM | 571 | HG1 | LYS | 360 | −4.865 | −2.922 | −28.539 | 0.00 | 0.00 | 0.053 | 0.00 | 0.00 |
| ATOM | 572 | HG2 | LYS | 360 | −3.756 | −2.853 | −29.888 | 0.00 | 0.00 | 0.053 | 0.00 | 0.00 |
| ATOM | 573 | CD | LYS | 360 | −5.711 | −3.492 | −30.397 | 0.00 | 0.00 | −0.106 | 12.77 | 4.00 |
| ATOM | 574 | HD1 | LYS | 360 | −5.759 | −3.096 | −31.411 | 0.00 | 0.00 | 0.053 | 0.00 | 0.00 |
| ATOM | 575 | HD2 | LYS | 360 | −6.696 | −3.471 | −29.930 | 0.00 | 0.00 | 0.053 | 0.00 | 0.00 |
| ATOM | 576 | CE | LYS | 360 | −5.271 | −4.953 | −30.489 | 0.00 | 0.00 | 0.099 | 12.77 | 4.00 |
| ATOM | 577 | HE1 | LYS | 360 | −5.399 | −5.441 | −29.523 | 0.00 | 0.00 | 0.053 | 0.00 | 0.00 |
| ATOM | 578 | HE2 | LYS | 360 | −4.222 | −5.007 | −30.778 | 0.00 | 0.00 | 0.053 | 0.00 | 0.00 |
| ATOM | 579 | NZ | LYS | 360 | −6.088 | −5.663 | −31.500 | 0.00 | 0.00 | −0.045 | 13.25 | −39.20 |
| ATOM | 580 | HZ1 | LYS | 360 | −5.786 | −6.646 | −31.557 | 0.00 | 0.00 | 0.280 | 0.00 | 0.00 |
| ATOM | 581 | HZ2 | LYS | 360 | −7.081 | −5.624 | −31.229 | 0.00 | 0.00 | 0.280 | 0.00 | 0.00 |
| ATOM | 582 | HZ3 | LYS | 360 | −5.966 | −5.213 | −32.418 | 0.00 | 0.00 | 0.280 | 0.00 | 0.00 |
| ATOM | 583 | N | THR | 361 | −2.405 | 0.327 | −30.220 | 0.00 | 0.00 | −0.650 | 9.00 | −17.40 |
| ATOM | 584 | HN | THR | 361 | −3.053 | 0.916 | −30.763 | 0.00 | 0.00 | 0.439 | 0.00 | 0.00 |
| ATOM | 585 | CA | THR | 361 | −1.048 | 0.199 | −30.661 | 0.00 | 0.00 | 0.158 | 9.40 | 4.00 |
| ATOM | 586 | HA | THR | 361 | −0.790 | −0.849 | −30.817 | 0.00 | 0.00 | 0.053 | 0.00 | 0.00 |
| ATOM | 587 | C | THR | 361 | −0.104 | 0.767 | −29.644 | 0.00 | 0.00 | 0.396 | 9.82 | 4.00 |
| ATOM | 588 | O | THR | 361 | 0.890 | 0.132 | −29.292 | 0.00 | 0.00 | −0.396 | 8.17 | −17.40 |
| ATOM | 589 | CB | THR | 361 | −0.827 | 0.917 | −31.964 | 0.00 | 0.00 | 0.060 | 9.40 | 4.00 |
| ATOM | 590 | HB | THR | 361 | 0.204 | 0.753 | −32.278 | 0.00 | 0.00 | 0.053 | 0.00 | 0.00 |
| ATOM | 591 | OG1 | THR | 361 | −1.066 | 2.308 | −31.805 | 0.00 | 0.00 | −0.537 | 11.04 | −17.40 |
| ATOM | 592 | HG1 | THR | 361 | −0.173 | 2.819 | −31.858 | 0.00 | 0.00 | 0.424 | 0.00 | 0.00 |
| ATOM | 593 | CG2 | THR | 361 | −1.805 | 0.346 | −33.007 | 0.00 | 0.00 | −0.159 | 16.15 | 4.00 |
| ATOM | 594 | HG21 | THR | 361 | −1.659 | 0.855 | −33.960 | 0.00 | 0.00 | 0.053 | 0.00 | 0.00 |
| ATOM | 595 | HG22 | THR | 361 | −1.620 | −0.721 | −33.134 | 0.00 | 0.00 | 0.053 | 0.00 | 0.00 |
| ATOM | 596 | HG23 | THR | 361 | −2.829 | 0.499 | −32.667 | 0.00 | 0.00 | 0.053 | 0.00 | 0.00 |
| ATOM | 597 | N | LEU | 362 | −0.389 | 1.980 | −29.138 | 0.00 | 0.00 | −0.650 | 9.00 | −17.40 |
| ATOM | 598 | HN | LEU | 362 | −1.261 | 2.456 | −29.410 | 0.00 | 0.00 | 0.439 | 0.00 | 0.00 |
| ATOM | 599 | CA | LEU | 362 | 0.515 | 2.615 | −28.221 | 0.00 | 0.00 | 0.158 | 9.40 | 4.00 |
| ATOM | 600 | HA | LEU | 362 | 1.527 | 2.638 | −28.624 | 0.00 | 0.00 | 0.053 | 0.00 | 0.00 |
| ATOM | 601 | C | LEU | 362 | 0.556 | 1.881 | −26.911 | 0.00 | 0.00 | 0.396 | 9.82 | 4.00 |
| ATOM | 602 | O | LEU | 362 | 1.633 | 1.683 | −26.345 | 0.00 | 0.00 | −0.396 | 8.17 | −17.40 |
| ATOM | 603 | CB | LEU | 362 | 0.146 | 4.085 | −27.946 | 0.00 | 0.00 | −0.106 | 12.77 | 4.00 |
| ATOM | 604 | HB1 | LEU | 362 | 0.866 | 4.492 | −27.236 | 0.00 | 0.00 | 0.053 | 0.00 | 0.00 |
| ATOM | 605 | HB2 | LEU | 362 | −0.861 | 4.114 | −27.530 | 0.00 | 0.00 | 0.053 | 0.00 | 0.00 |
| ATOM | 606 | CG | LEU | 362 | 0.162 | 4.984 | −29.197 | 0.00 | 0.00 | −0.053 | 9.40 | 4.00 |
| ATOM | 607 | HG | LEU | 362 | 1.118 | 4.928 | −29.718 | 0.00 | 0.00 | 0.053 | 0.00 | 0.00 |
| ATOM | 608 | CD1 | LEU | 362 | −0.929 | 4.576 | −30.198 | 0.00 | 0.00 | −0.159 | 16.15 | 4.00 |
| ATOM | 609 | HD11 | LEU | 362 | −0.888 | 5.231 | −31.068 | 0.00 | 0.00 | 0.053 | 0.00 | 0.00 |
| ATOM | 610 | HD12 | LEU | 362 | −0.767 | 3.545 | −30.513 | 0.00 | 0.00 | 0.053 | 0.00 | 0.00 |
| ATOM | 611 | HD13 | LEU | 362 | −1.907 | 4.661 | −29.725 | 0.00 | 0.00 | 0.053 | 0.00 | 0.00 |
| ATOM | 612 | CD2 | LEU | 362 | 0.070 | 6.467 | −28.812 | 0.00 | 0.00 | −0.159 | 16.15 | 4.00 |
| ATOM | 613 | HD21 | LEU | 362 | 0.083 | 7.079 | −29.714 | 0.00 | 0.00 | 0.053 | 0.00 | 0.00 |
| ATOM | 614 | HD22 | LEU | 362 | −0.857 | 6.645 | −28.267 | 0.00 | 0.00 | 0.053 | 0.00 | 0.00 |
| ATOM | 615 | HD23 | LEU | 362 | 0.918 | 6.732 | −28.181 | 0.00 | 0.00 | 0.053 | 0.00 | 0.00 |
| ATOM | 616 | N | GLY | 363 | −0.607 | 1.442 | −26.382 | 0.00 | 0.00 | −0.650 | 9.00 | −17.40 |
| ATOM | 617 | HN | GLY | 363 | −1.503 | 1.564 | −26.876 | 0.00 | 0.00 | 0.439 | 0.00 | 0.00 |
| ATOM | 618 | CA | GLY | 363 | −0.539 | 0.797 | −25.100 | 0.00 | 0.00 | 0.105 | 9.40 | 4.00 |
| ATOM | 619 | HA1 | GLY | 363 | −1.015 | 1.407 | −24.332 | 0.00 | 0.00 | 0.053 | 0.00 | 0.00 |
| ATOM | 620 | HA2 | GLY | 363 | 0.496 | 0.629 | −24.803 | 0.00 | 0.00 | 0.053 | 0.00 | 0.00 |
| ATOM | 621 | C | GLY | 363 | −1.232 | −0.530 | −25.132 | 0.00 | 0.00 | 0.396 | 9.82 | 4.00 |
| ATOM | 622 | O | GLY | 363 | −2.401 | −0.648 | −24.770 | 0.00 | 0.00 | −0.396 | 8.17 | −17.40 |
| ATOM | 623 | N | PRO | 364 | −0.520 | −1.555 | −25.510 | 0.00 | 0.00 | −0.422 | 9.00 | −17.40 |
| ATOM | 624 | CA | PRO | 364 | −1.149 | −2.844 | −25.515 | 0.00 | 0.00 | 0.158 | 9.40 | 4.00 |
| ATOM | 625 | HA | PRO | 364 | −2.138 | −2.766 | −25.966 | 0.00 | 0.00 | 0.053 | 0.00 | 0.00 |
| ATOM | 626 | CD | PRO | 364 | 0.461 | −1.427 | −26.575 | 0.00 | 0.00 | 0.105 | 12.77 | 4.00 |
| ATOM | 627 | HD1 | PRO | 364 | 1.422 | −1.326 | −26.072 | 0.00 | 0.00 | 0.053 | 0.00 | 0.00 |
| ATOM | 628 | HD2 | PRO | 364 | 0.170 | −0.535 | −27.130 | 0.00 | 0.00 | 0.053 | 0.00 | 0.00 |
| ATOM | 629 | C | PRO | 364 | −1.281 | −3.341 | −24.114 | 0.00 | 0.00 | 0.396 | 9.82 | 4.00 |
| ATOM | 630 | O | PRO | 364 | −0.393 | −3.080 | −23.302 | 0.00 | 0.00 | −0.396 | 8.17 | −17.40 |
| ATOM | 631 | CB | PRO | 364 | −0.324 | −3.727 | −26.441 | 0.00 | 0.00 | −0.106 | 12.77 | 4.00 |
| ATOM | 632 | HB1 | PRO | 364 | −0.959 | −4.434 | −26.975 | 0.00 | 0.00 | 0.053 | 0.00 | 0.00 |
| ATOM | 633 | HB2 | PRO | 364 | 0.415 | −4.299 | −25.880 | 0.00 | 0.00 | 0.053 | 0.00 | 0.00 |
| ATOM | 634 | CG | PRO | 364 | 0.340 | −2.720 | −27.398 | 0.00 | 0.00 | −0.106 | 12.77 | 4.00 |
| ATOM | 635 | HG1 | PRO | 364 | −0.350 | −2.646 | −28.239 | 0.00 | 0.00 | 0.053 | 0.00 | 0.00 |
| ATOM | 636 | HG2 | PRO | 364 | 1.298 | −3.174 | −27.651 | 0.00 | 0.00 | 0.053 | 0.00 | 0.00 |
| ATOM | 637 | N | SER | 365 | −2.370 | −4.067 | −23.804 | 0.00 | 0.00 | −0.650 | 9.00 | −17.40 |
| ATOM | 638 | HN | SER | 365 | −3.067 | −4.308 | −24.523 | 0.00 | 0.00 | 0.439 | 0.00 | 0.00 |
| ATOM | 639 | CA | SER | 365 | −2.536 | −4.496 | −22.449 | 0.00 | 0.00 | 0.158 | 9.40 | 4.00 |
| ATOM | 640 | HA | SER | 365 | −1.844 | −3.950 | −21.808 | 0.00 | 0.00 | 0.053 | 0.00 | 0.00 |
| ATOM | 641 | C | SER | 365 | −2.255 | −5.961 | −22.352 | 0.00 | 0.00 | 0.396 | 9.82 | 4.00 |
| ATOM | 642 | O | SER | 365 | −2.814 | −6.776 | −23.089 | 0.00 | 0.00 | −0.396 | 8.17 | −17.40 |
| ATOM | 643 | CB | SER | 365 | −3.953 | −4.270 | −21.895 | 0.00 | 0.00 | 0.007 | 12.77 | 4.00 |
| ATOM | 644 | HB1 | SER | 365 | −4.676 | −4.821 | −22.496 | 0.00 | 0.00 | 0.053 | 0.00 | 0.00 |
| ATOM | 645 | HB2 | SER | 365 | −4.197 | −3.208 | −21.927 | 0.00 | 0.00 | 0.053 | 0.00 | 0.00 |
| ATOM | 646 | OG | SER | 365 | −4.022 | −4.724 | −20.550 | 0.00 | 0.00 | −0.537 | 11.04 | −17.40 |
| ATOM | 647 | HG | SER | 365 | −4.349 | −3.957 | −19.945 | 0.00 | 0.00 | 0.424 | 0.00 | 0.00 |
| ATOM | 648 | N | TRP | 366 | −1.345 | −6.345 | −21.437 | 0.00 | 0.00 | −0.650 | 9.00 | −17.40 |
| ATOM | 649 | HN | TRP | 366 | −0.811 | −5.664 | −20.879 | 0.00 | 0.00 | 0.439 | 0.00 | 0.00 |
| ATOM | 650 | CA | TRP | 366 | −1.166 | −7.751 | −21.288 | 0.00 | 0.00 | 0.158 | 9.40 | 4.00 |
| ATOM | 651 | HA | TRP | 366 | −1.747 | −8.300 | −22.029 | 0.00 | 0.00 | 0.053 | 0.00 | 0.00 |
| ATOM | 652 | C | TRP | 366 | −1.648 | −8.013 | −19.900 | 0.00 | 0.00 | 0.396 | 9.82 | 4.00 |
| ATOM | 653 | O | TRP | 366 | −1.330 | −7.281 | −18.961 | 0.00 | 0.00 | −0.396 | 8.17 | −17.40 |
| ATOM | 654 | CB | TRP | 366 | 0.281 | −8.257 | −21.369 | 0.00 | 0.00 | −0.106 | 12.77 | 4.00 |
| ATOM | 655 | HB1 | TRP | 366 | 0.919 | −7.621 | −21.982 | 0.00 | 0.00 | 0.053 | 0.00 | 0.00 |
| ATOM | 656 | HB2 | TRP | 366 | 0.358 | −9.256 | −21.798 | 0.00 | 0.00 | 0.053 | 0.00 | 0.00 |
| ATOM | 657 | CG | TRP | 366 | 0.948 | −8.335 | −20.028 | 0.00 | 0.00 | 0.000 | 7.26 | 0.60 |
| ATOM | 658 | CD1 | TRP | 366 | 1.466 | −7.357 | −19.241 | 0.00 | 0.00 | −0.177 | 10.80 | 0.60 |
| ATOM | 659 | HD1 | TRP | 366 | 1.461 | −6.293 | −19.480 | 0.00 | 0.00 | 0.127 | 0.00 | 0.00 |
| ATOM | 660 | CD2 | TRP | 366 | 1.154 | −9.572 | −19.337 | 0.00 | 0.00 | 0.000 | 6.80 | 0.60 |
| ATOM | 661 | NE1 | TRP | 366 | 1.998 | −7.907 | −18.099 | 0.00 | 0.00 | −0.292 | 9.00 | −17.40 |
| ATOM | 662 | HE1 | TRP | 366 | 2.459 | −7.389 | −17.338 | 0.00 | 0.00 | 0.393 | 0.00 | 0.00 |
| ATOM | 663 | CE2 | TRP | 366 | 1.809 | −9.270 | −18.148 | 0.00 | 0.00 | −0.050 | 6.80 | 0.60 |
| ATOM | 664 | CE3 | TRP | 366 | 0.825 | −10.856 | −19.666 | 0.00 | 0.00 | −0.127 | 10.80 | 0.60 |
| ATOM | 665 | HE3 | TRP | 366 | 0.310 | −11.092 | −20.597 | 0.00 | 0.00 | 0.127 | 0.00 | 0.00 |
| ATOM | 666 | CZ2 | TRP | 366 | 2.148 | −10.253 | −17.270 | 0.00 | 0.00 | −0.127 | 10.80 | 0.60 |
| ATOM | 667 | HZ2 | TRP | 366 | 2.661 | −10.020 | −16.337 | 0.00 | 0.00 | 0.127 | 0.00 | 0.00 |
| ATOM | 668 | CZ3 | TRP | 366 | 1.174 | −11.839 | −18.767 | 0.00 | 0.00 | −0.127 | 10.80 | 0.60 |
| ATOM | 669 | HZ3 | TRP | 366 | 0.927 | −12.876 | −18.993 | 0.00 | 0.00 | 0.127 | 0.00 | 0.00 |
| ATOM | 670 | CH2 | TRP | 366 | 1.824 | −11.548 | −17.594 | 0.00 | 0.00 | −0.127 | 10.80 | 0.60 |
| ATOM | 671 | HH2 | TRP | 366 | 2.087 | −12.355 | −16.910 | 0.00 | 0.00 | 0.127 | 0.00 | 0.00 |
| ATOM | 672 | N | VAL | 367 | −2.473 | −9.053 | −19.722 | 0.00 | 0.00 | −0.650 | 9.00 | −17.40 |
| ATOM | 673 | HN | VAL | 367 | −2.740 | −9.677 | −20.496 | 0.00 | 0.00 | 0.439 | 0.00 | 0.00 |
| ATOM | 674 | CA | VAL | 367 | −2.958 | −9.243 | −18.397 | 0.00 | 0.00 | 0.158 | 9.40 | 4.00 |
| ATOM | 675 | HA | VAL | 367 | −2.673 | −8.365 | −17.818 | 0.00 | 0.00 | 0.053 | 0.00 | 0.00 |
| ATOM | 676 | C | VAL | 367 | −2.317 | −10.487 | −17.861 | 0.00 | 0.00 | 0.396 | 9.82 | 4.00 |
| ATOM | 677 | O | VAL | 367 | −2.369 | −11.557 | −18.468 | 0.00 | 0.00 | −0.396 | 8.17 | −17.40 |
| ATOM | 678 | CB | VAL | 367 | −4.462 | −9.327 | −18.328 | 0.00 | 0.00 | −0.053 | 9.40 | 4.00 |
| ATOM | 679 | HB | VAL | 367 | −4.918 | −8.573 | −18.970 | 0.00 | 0.00 | 0.053 | 0.00 | 0.00 |
| ATOM | 680 | CG1 | VAL | 367 | −4.947 | −10.709 | −18.787 | 0.00 | 0.00 | −0.159 | 16.15 | 4.00 |
| ATOM | 681 | HG11 | VAL | 367 | −6.035 | −10.750 | −18.730 | 0.00 | 0.00 | 0.053 | 0.00 | 0.00 |
| ATOM | 682 | HG12 | VAL | 367 | −4.631 | −10.883 | −19.816 | 0.00 | 0.00 | 0.053 | 0.00 | 0.00 |
| ATOM | 683 | HG13 | VAL | 367 | −4.521 | −11.477 | −18.142 | 0.00 | 0.00 | 0.053 | 0.00 | 0.00 |
| ATOM | 684 | CG2 | VAL | 367 | −4.904 | −8.914 | −16.921 | 0.00 | 0.00 | −0.159 | 16.15 | 4.00 |
| ATOM | 685 | HG21 | VAL | 367 | −5.990 | −8.968 | −16.850 | 0.00 | 0.00 | 0.053 | 0.00 | 0.00 |
| ATOM | 686 | HG22 | VAL | 367 | −4.459 | −9.586 | −16.188 | 0.00 | 0.00 | 0.053 | 0.00 | 0.00 |
| ATOM | 687 | HG23 | VAL | 367 | −4.578 | −7.893 | −16.722 | 0.00 | 0.00 | 0.053 | 0.00 | 0.00 |
| ATOM | 688 | N | HIS | 368 | −1.642 | −10.345 | −16.705 | 0.00 | 0.00 | −0.650 | 9.00 | −17.40 |
| ATOM | 689 | HN | HIS | 368 | −1.581 | −9.409 | −16.278 | 0.00 | 0.00 | 0.439 | 0.00 | 0.00 |
| ATOM | 690 | CA | HIS | 368 | −1.006 | −11.446 | −16.046 | 0.00 | 0.00 | 0.158 | 9.40 | 4.00 |
| ATOM | 691 | HA | HIS | 368 | −0.772 | −12.161 | −16.834 | 0.00 | 0.00 | 0.053 | 0.00 | 0.00 |
| ATOM | 692 | C | HIS | 368 | −2.004 | −11.956 | −15.074 | 0.00 | 0.00 | 0.396 | 9.82 | 4.00 |
| ATOM | 693 | O | HIS | 368 | −2.461 | −11.217 | −14.205 | 0.00 | 0.00 | −0.396 | 8.17 | −17.40 |
| ATOM | 694 | CB | HIS | 368 | 0.203 | −11.031 | −15.192 | 0.00 | 0.00 | −0.106 | 12.77 | 4.00 |
| ATOM | 695 | HB1 | HIS | 368 | 0.274 | −9.954 | −15.041 | 0.00 | 0.00 | 0.053 | 0.00 | 0.00 |
| ATOM | 696 | HB2 | HIS | 368 | 1.158 | −11.326 | −15.626 | 0.00 | 0.00 | 0.053 | 0.00 | 0.00 |
| ATOM | 697 | CG | HIS | 368 | 0.189 | −11.628 | −13.813 | 0.00 | 0.00 | 0.241 | 7.26 | 0.60 |
| ATOM | 698 | ND1 | HIS | 368 | −0.014 | −12.962 | −13.539 | 0.00 | 0.00 | −0.481 | 9.25 | −17.40 |
| ATOM | 699 | CD2 | HIS | 368 | 0.364 | −11.024 | −12.604 | 0.00 | 0.00 | −0.177 | 10.80 | 0.60 |
| ATOM | 700 | HD2 | HIS | 368 | 0.548 | −9.959 | −12.463 | 0.00 | 0.00 | 0.127 | 0.00 | 0.00 |
| ATOM | 701 | CE1 | HIS | 368 | 0.049 | −13.099 | −12.190 | 0.00 | 0.00 | 0.064 | 10.80 | 0.60 |
| ATOM | 702 | HE1 | HIS | 368 | −0.074 | −14.049 | −11.670 | 0.00 | 0.00 | 0.127 | 0.00 | 0.00 |
| ATOM | 703 | NE2 | HIS | 368 | 0.276 | −11.950 | −11.580 | 0.00 | 0.00 | −0.292 | 9.25 | −17.40 |
| ATOM | 704 | HE2 | HIS | 368 | 0.367 | −11.781 | −10.568 | 0.00 | 0.00 | 0.393 | 0.00 | 0.00 |
| ATOM | 705 | N | TYR | 369 | −2.379 | −13.240 | −15.183 | 0.00 | 0.00 | −0.650 | 9.00 | −17.40 |
| ATOM | 706 | HN | TYR | 369 | −1.970 | −13.866 | −15.891 | 0.00 | 0.00 | 0.439 | 0.00 | 0.00 |
| ATOM | 707 | CA | TYR | 369 | −3.376 | −13.694 | −14.265 | 0.00 | 0.00 | 0.158 | 9.40 | 4.00 |
| ATOM | 708 | HA | TYR | 369 | −3.646 | −12.873 | −13.600 | 0.00 | 0.00 | 0.053 | 0.00 | 0.00 |
| ATOM | 709 | C | TYR | 369 | −2.824 | −14.831 | −13.472 | 0.00 | 0.00 | 0.396 | 9.82 | 4.00 |
| ATOM | 710 | O | TYR | 369 | −2.335 | −15.816 | −14.024 | 0.00 | 0.00 | −0.396 | 8.17 | −17.40 |
| ATOM | 711 | CB | TYR | 369 | −4.662 | −14.140 | −14.988 | 0.00 | 0.00 | −0.106 | 12.77 | 4.00 |
| ATOM | 712 | HB1 | TYR | 369 | −4.449 | −14.978 | −15.652 | 0.00 | 0.00 | 0.053 | 0.00 | 0.00 |
| ATOM | 713 | HB2 | TYR | 369 | −5.066 | −13.318 | −15.579 | 0.00 | 0.00 | 0.053 | 0.00 | 0.00 |
| ATOM | 714 | CG | TYR | 369 | −5.689 | −14.565 | −13.996 | 0.00 | 0.00 | 0.000 | 7.26 | 0.60 |
| ATOM | 715 | CD1 | TYR | 369 | −6.254 | −13.646 | −13.142 | 0.00 | 0.00 | −0.127 | 10.80 | 0.60 |
| ATOM | 716 | HD1 | TYR | 369 | −5.938 | −12.604 | −13.186 | 0.00 | 0.00 | 0.127 | 0.00 | 0.00 |
| ATOM | 717 | CD2 | TYR | 369 | −6.118 | −15.871 | −13.950 | 0.00 | 0.00 | −0.127 | 10.80 | 0.60 |
| ATOM | 718 | HD2 | TYR | 369 | −5.694 | −16.604 | −14.637 | 0.00 | 0.00 | 0.127 | 0.00 | 0.00 |
| ATOM | 719 | CE1 | TYR | 369 | −7.213 | −14.029 | −12.234 | 0.00 | 0.00 | −0.127 | 10.80 | 0.60 |
| ATOM | 720 | HE1 | TYR | 369 | −7.646 | −13.295 | −11.555 | 0.00 | 0.00 | 0.127 | 0.00 | 0.00 |
| ATOM | 721 | CE2 | TYR | 369 | −7.077 | −16.259 | −13.045 | 0.00 | 0.00 | −0.127 | 10.80 | 0.60 |
| ATOM | 722 | HE2 | TYR | 369 | −7.404 | −17.298 | −13.010 | 0.00 | 0.00 | 0.127 | 0.00 | 0.00 |
| ATOM | 723 | CZ | TYR | 369 | −7.624 | −15.339 | −12.184 | 0.00 | 0.00 | 0.027 | 7.26 | 0.60 |
| ATOM | 724 | OH | TYR | 369 | −8.609 | −15.742 | −11.259 | 0.00 | 0.00 | −0.451 | 10.94 | −17.40 |
| ATOM | 725 | HH | TYR | 369 | −9.127 | −14.916 | −10.926 | 0.00 | 0.00 | 0.424 | 0.00 | 0.00 |
| ATOM | 726 | N | GLN | 370 | −2.875 | −14.699 | −12.131 | 0.00 | 0.00 | −0.650 | 9.00 | −17.40 |
| ATOM | 727 | HN | GLN | 370 | −3.249 | −13.830 | −11.724 | 0.00 | 0.00 | 0.439 | 0.00 | 0.00 |
| ATOM | 728 | CA | GLN | 370 | −2.420 | −15.741 | −11.259 | 0.00 | 0.00 | 0.158 | 9.40 | 4.00 |
| ATOM | 729 | HA | GLN | 370 | −1.593 | −16.212 | −11.790 | 0.00 | 0.00 | 0.053 | 0.00 | 0.00 |
| ATOM | 730 | C | GLN | 370 | −3.599 | −16.636 | −11.079 | 0.00 | 0.00 | 0.396 | 9.82 | 4.00 |
| ATOM | 731 | O | GLN | 370 | −4.641 | −16.421 | −11.695 | 0.00 | 0.00 | −0.396 | 8.17 | −17.40 |
| ATOM | 732 | CB | GLN | 370 | −2.016 | −15.244 | −9.856 | 0.00 | 0.00 | −0.106 | 12.77 | 4.00 |
| ATOM | 733 | HB1 | GLN | 370 | −2.917 | −14.924 | −9.332 | 0.00 | 0.00 | 0.053 | 0.00 | 0.00 |
| ATOM | 734 | HB2 | GLN | 370 | −1.325 | −14.409 | −9.973 | 0.00 | 0.00 | 0.053 | 0.00 | 0.00 |
| ATOM | 735 | CG | GLN | 370 | −1.325 | −16.308 | −8.995 | 0.00 | 0.00 | −0.106 | 12.77 | 4.00 |
| ATOM | 736 | HG1 | GLN | 370 | −0.299 | −16.428 | −9.344 | 0.00 | 0.00 | 0.053 | 0.00 | 0.00 |
| ATOM | 737 | HG2 | GLN | 370 | −1.870 | −17.247 | −9.096 | 0.00 | 0.00 | 0.053 | 0.00 | 0.00 |
| ATOM | 738 | CD | GLN | 370 | −1.337 | −15.845 | −7.545 | 0.00 | 0.00 | 0.396 | 9.82 | 4.00 |
| ATOM | 739 | OE1 | GLN | 370 | −1.929 | −14.819 | −7.212 | 0.00 | 0.00 | −0.396 | 8.17 | −17.40 |
| ATOM | 740 | NE2 | GLN | 370 | −0.670 | −16.627 | −6.654 | 0.00 | 0.00 | −0.879 | 13.25 | −17.40 |
| ATOM | 741 | HE21 | GLN | 370 | −0.188 | −17.478 | −6.976 | 0.00 | 0.00 | 0.439 | 0.00 | 0.00 |
| ATOM | 742 | HE22 | GLN | 370 | −0.647 | −16.368 | −5.657 | 0.00 | 0.00 | 0.439 | 0.00 | 0.00 |
| ATOM | 743 | N | CYS | 371 | −3.472 | −17.658 | −10.208 | 0.00 | 0.00 | −0.650 | 9.00 | −17.40 |
| ATOM | 744 | HN | CYS | 371 | −2.582 | −17.767 | −9.700 | 0.00 | 0.00 | 0.439 | 0.00 | 0.00 |
| ATOM | 745 | CA | CYS | 371 | −4.525 | −18.599 | −9.960 | 0.00 | 0.00 | 0.158 | 9.40 | 4.00 |
| ATOM | 746 | HA | CYS | 371 | −4.140 | −19.450 | −9.398 | 0.00 | 0.00 | 0.053 | 0.00 | 0.00 |
| ATOM | 747 | C | CYS | 371 | −5.091 | −19.089 | −11.258 | 0.00 | 0.00 | 0.396 | 9.82 | 4.00 |
| ATOM | 748 | O | CYS | 371 | −4.542 | −18.870 | −12.335 | 0.00 | 0.00 | −0.396 | 8.17 | −17.40 |
| ATOM | 749 | CB | CYS | 371 | −5.667 | −18.012 | −9.115 | 0.00 | 0.00 | −0.041 | 12.77 | 4.00 |
| ATOM | 750 | HB1 | CYS | 371 | −6.480 | −18.726 | −8.984 | 0.00 | 0.00 | 0.053 | 0.00 | 0.00 |
| ATOM | 751 | HB2 | CYS | 371 | −6.093 | −17.121 | −9.577 | 0.00 | 0.00 | 0.053 | 0.00 | 0.00 |
| ATOM | 752 | SG | CYS | 371 | −5.099 | −17.536 | −7.456 | 0.00 | 0.00 | −0.255 | 19.93 | −6.40 |
| ATOM | 753 | HG | CYS | 371 | −4.628 | −16.282 | −7.487 | 0.00 | 0.00 | 0.190 | 0.00 | 0.00 |
| ATOM | 754 | N | MET | 372 | −6.186 | −19.859 | −11.188 | 0.00 | 0.00 | −0.650 | 9.00 | −17.40 |
| ATOM | 755 | HN | MET | 372 | −6.624 | −20.085 | −10.284 | 0.00 | 0.00 | 0.439 | 0.00 | 0.00 |
| ATOM | 756 | CA | MET | 372 | −6.723 | −20.355 | −12.418 | 0.00 | 0.00 | 0.158 | 9.40 | 4.00 |
| ATOM | 757 | HA | MET | 372 | −6.234 | −19.834 | −13.241 | 0.00 | 0.00 | 0.053 | 0.00 | 0.00 |
| ATOM | 758 | C | MET | 372 | −8.193 | −20.091 | −12.430 | 0.00 | 0.00 | 0.396 | 9.82 | 4.00 |
| ATOM | 759 | O | MET | 372 | −8.972 | −20.827 | −11.827 | 0.00 | 0.00 | −0.396 | 8.17 | −17.40 |
| ATOM | 760 | CB | MET | 372 | −6.523 | −21.869 | −12.596 | 0.00 | 0.00 | −0.106 | 12.77 | 4.00 |
| ATOM | 761 | HB1 | MET | 372 | −7.001 | −22.468 | −11.821 | 0.00 | 0.00 | 0.053 | 0.00 | 0.00 |
| ATOM | 762 | HB2 | MET | 372 | −5.477 | −22.177 | −12.584 | 0.00 | 0.00 | 0.053 | 0.00 | 0.00 |
| ATOM | 763 | CG | MET | 372 | −7.079 | −22.411 | −13.913 | 0.00 | 0.00 | −0.041 | 12.77 | 4.00 |
| ATOM | 764 | HG1 | MET | 372 | −6.602 | −21.874 | −14.732 | 0.00 | 0.00 | 0.053 | 0.00 | 0.00 |
| ATOM | 765 | HG2 | MET | 372 | −8.157 | −22.247 | −13.922 | 0.00 | 0.00 | 0.053 | 0.00 | 0.00 |
| ATOM | 766 | SD | MET | 372 | −6.790 | −24.186 | −14.177 | 0.00 | 0.00 | −0.130 | 16.39 | −6.40 |
| ATOM | 767 | CE | MET | 372 | −7.914 | −24.727 | −12.857 | 0.00 | 0.00 | −0.094 | 16.15 | 4.00 |
| ATOM | 768 | HE1 | MET | 372 | −7.920 | −25.816 | −12.808 | 0.00 | 0.00 | 0.053 | 0.00 | 0.00 |
| ATOM | 769 | HE2 | MET | 372 | −7.576 | −24.322 | −11.903 | 0.00 | 0.00 | 0.053 | 0.00 | 0.00 |
| ATOM | 770 | HE3 | MET | 372 | −8.922 | −24.368 | −13.067 | 0.00 | 0.00 | 0.053 | 0.00 | 0.00 |
| ATOM | 771 | N | LEU | 373 | −8.604 | −19.008 | −13.116 | 0.00 | 0.00 | −0.650 | 9.00 | −17.40 |
| ATOM | 772 | HN | LEU | 373 | −7.906 | −18.394 | −13.559 | 0.00 | 0.00 | 0.439 | 0.00 | 0.00 |
| ATOM | 773 | CA | LEU | 373 | −9.998 | −18.696 | −13.240 | 0.00 | 0.00 | 0.158 | 9.40 | 4.00 |
| ATOM | 774 | HA | LEU | 373 | −10.532 | −19.636 | −13.103 | 0.00 | 0.00 | 0.053 | 0.00 | 0.00 |
| ATOM | 775 | C | LEU | 373 | −10.176 | −18.128 | −14.612 | 0.00 | 0.00 | 0.396 | 9.82 | 4.00 |
| ATOM | 776 | O | LEU | 373 | −9.200 | −17.914 | −15.328 | 0.00 | 0.00 | −0.396 | 8.17 | −17.40 |
| ATOM | 777 | CB | LEU | 373 | −10.504 | −17.643 | −12.238 | 0.00 | 0.00 | −0.106 | 12.77 | 4.00 |
| ATOM | 778 | HB1 | LEU | 373 | −11.535 | −17.396 | −12.492 | 0.00 | 0.00 | 0.053 | 0.00 | 0.00 |
| ATOM | 779 | HB2 | LEU | 373 | −9.868 | −16.762 | −12.317 | 0.00 | 0.00 | 0.053 | 0.00 | 0.00 |
| ATOM | 780 | CG | LEU | 373 | −10.482 | −18.108 | −10.767 | 0.00 | 0.00 | −0.053 | 9.40 | 4.00 |
| ATOM | 781 | HG | LEU | 373 | −11.000 | −19.058 | −10.634 | 0.00 | 0.00 | 0.053 | 0.00 | 0.00 |
| ATOM | 782 | CD1 | LEU | 373 | −9.048 | −18.302 | −10.250 | 0.00 | 0.00 | −0.159 | 16.15 | 4.00 |
| ATOM | 783 | HD11 | LEU | 373 | −9.077 | −18.630 | −9.211 | 0.00 | 0.00 | 0.053 | 0.00 | 0.00 |
| ATOM | 784 | HD12 | LEU | 373 | −8.542 | −19.055 | −10.854 | 0.00 | 0.00 | 0.053 | 0.00 | 0.00 |
| ATOM | 785 | HD13 | LEU | 373 | −8.506 | −17.359 | −10.318 | 0.00 | 0.00 | 0.053 | 0.00 | 0.00 |
| ATOM | 786 | CD2 | LEU | 373 | −11.314 | −17.172 | −9.875 | 0.00 | 0.00 | −0.159 | 16.15 | 4.00 |
| ATOM | 787 | HD21 | LEU | 373 | −11.279 | −17.526 | −8.845 | 0.00 | 0.00 | 0.053 | 0.00 | 0.00 |
| ATOM | 788 | HD22 | LEU | 373 | −10.906 | −16.163 | −9.926 | 0.00 | 0.00 | 0.053 | 0.00 | 0.00 |
| ATOM | 789 | HD23 | LEU | 373 | −12.348 | −17.163 | −10.221 | 0.00 | 0.00 | 0.053 | 0.00 | 0.00 |
| ATOM | 790 | N | ARG | 374 | −11.438 | −17.890 | −15.032 | 0.00 | 0.00 | −0.650 | 9.00 | −17.40 |
| ATOM | 791 | HN | ARG | 374 | −12.238 | −18.103 | −14.420 | 0.00 | 0.00 | 0.439 | 0.00 | 0.00 |
| ATOM | 792 | CA | ARG | 374 | −11.653 | −17.337 | −16.339 | 0.00 | 0.00 | 0.158 | 9.40 | 4.00 |
| ATOM | 793 | HA | ARG | 374 | −10.681 | −17.105 | −16.775 | 0.00 | 0.00 | 0.053 | 0.00 | 0.00 |
| ATOM | 794 | C | ARG | 374 | −12.477 | −16.095 | −16.192 | 0.00 | 0.00 | 0.396 | 9.82 | 4.00 |
| ATOM | 795 | O | ARG | 374 | −13.170 | −15.925 | −15.190 | 0.00 | 0.00 | −0.396 | 8.17 | −17.40 |
| ATOM | 796 | CB | ARG | 374 | −12.414 | −18.272 | −17.295 | 0.00 | 0.00 | −0.106 | 12.77 | 4.00 |
| ATOM | 797 | HB1 | ARG | 374 | −11.917 | −19.228 | −17.460 | 0.00 | 0.00 | 0.053 | 0.00 | 0.00 |
| ATOM | 798 | HB2 | ARG | 374 | −12.559 | −17.855 | −18.292 | 0.00 | 0.00 | 0.053 | 0.00 | 0.00 |
| ATOM | 799 | CG | ARG | 374 | −13.819 | −18.635 | −16.810 | 0.00 | 0.00 | −0.106 | 12.77 | 4.00 |
| ATOM | 800 | HG1 | ARG | 374 | −14.458 | −17.769 | −16.637 | 0.00 | 0.00 | 0.053 | 0.00 | 0.00 |
| ATOM | 801 | HG2 | ARG | 374 | −13.827 | −19.186 | −15.869 | 0.00 | 0.00 | 0.053 | 0.00 | 0.00 |
| ATOM | 802 | CD | ARG | 374 | −14.598 | −19.514 | −17.792 | 0.00 | 0.00 | 0.374 | 12.77 | 4.00 |
| ATOM | 803 | HD1 | ARG | 374 | −14.546 | −19.041 | −18.773 | 0.00 | 0.00 | 0.053 | 0.00 | 0.00 |
| ATOM | 804 | HD2 | ARG | 374 | −15.627 | −19.575 | −17.438 | 0.00 | 0.00 | 0.053 | 0.00 | 0.00 |
| ATOM | 805 | NE | ARG | 374 | −13.954 | −20.857 | −17.807 | 0.00 | 0.00 | −0.819 | 9.00 | −24.67 |
| ATOM | 806 | HE | ARG | 374 | −13.053 | −21.003 | −17.329 | 0.00 | 0.00 | 0.407 | 0.00 | 0.00 |
| ATOM | 807 | CZ | ARG | 374 | −14.559 | −21.894 | −18.455 | 0.00 | 0.00 | 0.796 | 6.95 | 4.00 |
| ATOM | 808 | NH1 | ARG | 374 | −15.748 | −21.699 | −19.096 | 0.00 | 0.00 | −0.746 | 9.00 | −24.67 |
| ATOM | 809 | HH11 | ARG | 374 | −16.204 | −22.483 | −19.585 | 0.00 | 0.00 | 0.407 | 0.00 | 0.00 |
| ATOM | 810 | HH12 | ARG | 374 | −16.189 | −20.768 | −19.091 | 0.00 | 0.00 | 0.407 | 0.00 | 0.00 |
| ATOM | 811 | NH2 | ARG | 374 | −13.976 | −23.129 | −18.462 | 0.00 | 0.00 | −0.746 | 9.00 | −24.67 |
| ATOM | 812 | HH21 | ARG | 374 | −14.433 | −23.912 | −18.951 | 0.00 | 0.00 | 0.407 | 0.00 | 0.00 |
| ATOM | 813 | HH22 | ARG | 374 | −13.079 | −23.278 | −17.978 | 0.00 | 0.00 | 0.407 | 0.00 | 0.00 |
| ATOM | 814 | N | VAL | 375 | −12.403 | −15.187 | −17.194 | 0.00 | 0.00 | −0.650 | 9.00 | −17.40 |
| ATOM | 815 | HN | VAL | 375 | −11.787 | −15.376 | −17.997 | 0.00 | 0.00 | 0.439 | 0.00 | 0.00 |
| ATOM | 816 | CA | VAL | 375 | −13.162 | −13.965 | −17.167 | 0.00 | 0.00 | 0.158 | 9.40 | 4.00 |
| ATOM | 817 | HA | VAL | 375 | −14.215 | −14.195 | −17.333 | 0.00 | 0.00 | 0.053 | 0.00 | 0.00 |
| ATOM | 818 | C | VAL | 375 | −12.662 | −13.050 | −18.253 | 0.00 | 0.00 | 0.396 | 9.82 | 4.00 |
| ATOM | 819 | O | VAL | 375 | −13.367 | −12.786 | −19.225 | 0.00 | 0.00 | −0.396 | 8.17 | −17.40 |
| ATOM | 820 | CB | VAL | 375 | −13.105 | −13.263 | −15.831 | 0.00 | 0.00 | −0.053 | 9.40 | 4.00 |
| ATOM | 821 | HB | VAL | 375 | −13.643 | −13.836 | −15.076 | 0.00 | 0.00 | 0.053 | 0.00 | 0.00 |
| ATOM | 822 | CG1 | VAL | 375 | −11.645 | −13.100 | −15.370 | 0.00 | 0.00 | −0.159 | 16.15 | 4.00 |
| ATOM | 823 | HG11 | VAL | 375 | −11.622 | −12.592 | −14.406 | 0.00 | 0.00 | 0.053 | 0.00 | 0.00 |
| ATOM | 824 | HG12 | VAL | 375 | −11.182 | −14.082 | −15.274 | 0.00 | 0.00 | 0.053 | 0.00 | 0.00 |
| ATOM | 825 | HG13 | VAL | 375 | −11.096 | −12.510 | −16.104 | 0.00 | 0.00 | 0.053 | 0.00 | 0.00 |
| ATOM | 826 | CG2 | VAL | 375 | −13.878 | −11.941 | −15.952 | 0.00 | 0.00 | −0.159 | 16.15 | 4.00 |
| ATOM | 827 | HG21 | VAL | 375 | −13.851 | −11.415 | −14.998 | 0.00 | 0.00 | 0.053 | 0.00 | 0.00 |
| ATOM | 828 | HG22 | VAL | 375 | −13.419 | −11.320 | −16.722 | 0.00 | 0.00 | 0.053 | 0.00 | 0.00 |
| ATOM | 829 | HG23 | VAL | 375 | −14.913 | −12.148 | −16.223 | 0.00 | 0.00 | 0.053 | 0.00 | 0.00 |
| ATOM | 830 | N | LEU | 376 | −11.418 | −12.556 | −18.118 | 0.00 | 0.00 | −0.650 | 9.00 | −17.40 |
| ATOM | 831 | HN | LEU | 376 | −10.876 | −12.842 | −17.290 | 0.00 | 0.00 | 0.439 | 0.00 | 0.00 |
| ATOM | 832 | CA | LEU | 376 | −10.794 | −11.657 | −19.049 | 0.00 | 0.00 | 0.158 | 9.40 | 4.00 |
| ATOM | 833 | HA | LEU | 376 | −11.480 | −10.817 | −19.159 | 0.00 | 0.00 | 0.053 | 0.00 | 0.00 |
| ATOM | 834 | C | LEU | 376 | −10.621 | −12.434 | −20.316 | 0.00 | 0.00 | 0.396 | 9.82 | 4.00 |
| ATOM | 835 | O | LEU | 376 | −10.578 | −11.885 | −21.416 | 0.00 | 0.00 | −0.396 | 8.17 | −17.40 |
| ATOM | 836 | CB | LEU | 376 | −9.439 | −11.136 | −18.520 | 0.00 | 0.00 | −0.106 | 12.77 | 4.00 |
| ATOM | 837 | HB1 | LEU | 376 | −8.727 | −11.960 | −18.561 | 0.00 | 0.00 | 0.053 | 0.00 | 0.00 |
| ATOM | 838 | HB2 | LEU | 376 | −9.590 | −10.798 | −17.495 | 0.00 | 0.00 | 0.053 | 0.00 | 0.00 |
| ATOM | 839 | CG | LEU | 376 | −8.806 | −9.956 | −19.294 | 0.00 | 0.00 | −0.053 | 9.40 | 4.00 |
| ATOM | 840 | HG | LEU | 376 | −9.524 | −9.144 | −19.410 | 0.00 | 0.00 | 0.053 | 0.00 | 0.00 |
| ATOM | 841 | CD1 | LEU | 376 | −7.581 | −9.414 | −18.544 | 0.00 | 0.00 | −0.159 | 16.15 | 4.00 |
| ATOM | 842 | HD11 | LEU | 376 | −7.149 | −8.584 | −19.104 | 0.00 | 0.00 | 0.053 | 0.00 | 0.00 |
| ATOM | 843 | HD12 | LEU | 376 | −7.884 | −9.067 | −17.556 | 0.00 | 0.00 | 0.053 | 0.00 | 0.00 |
| ATOM | 844 | HD13 | LEU | 376 | −6.839 | −10.206 | −18.439 | 0.00 | 0.00 | 0.053 | 0.00 | 0.00 |
| ATOM | 845 | CD2 | LEU | 376 | −8.448 | −10.313 | −20.743 | 0.00 | 0.00 | −0.159 | 16.15 | 4.00 |
| ATOM | 846 | HD21 | LEU | 376 | −8.008 | −9.445 | −21.234 | 0.00 | 0.00 | 0.053 | 0.00 | 0.00 |
| ATOM | 847 | HD22 | LEU | 376 | −7.732 | −11.135 | −20.749 | 0.00 | 0.00 | 0.053 | 0.00 | 0.00 |
| ATOM | 848 | HD23 | LEU | 376 | −9.349 | −10.613 | −21.277 | 0.00 | 0.00 | 0.053 | 0.00 | 0.00 |
| ATOM | 849 | N | ASP | 377 | −10.588 | −13.769 | −20.171 | 0.00 | 0.00 | −0.650 | 9.00 | −17.40 |
| ATOM | 850 | HN | ASP | 377 | −10.739 | −14.152 | −19.227 | 0.00 | 0.00 | 0.439 | 0.00 | 0.00 |
| ATOM | 851 | CA | ASP | 377 | −10.358 | −14.698 | −21.236 | 0.00 | 0.00 | 0.158 | 9.40 | 4.00 |
| ATOM | 852 | HA | ASP | 377 | −9.338 | −14.566 | −21.597 | 0.00 | 0.00 | 0.053 | 0.00 | 0.00 |
| ATOM | 853 | C | ASP | 377 | −11.336 | −14.419 | −22.329 | 0.00 | 0.00 | 0.396 | 9.82 | 4.00 |
| ATOM | 854 | O | ASP | 377 | −11.073 | −14.753 | −23.483 | 0.00 | 0.00 | −0.396 | 8.17 | −17.40 |
| ATOM | 855 | CB | ASP | 377 | −10.556 | −16.160 | −20.794 | 0.00 | 0.00 | −0.336 | 12.77 | 4.00 |
| ATOM | 856 | HB1 | ASP | 377 | −11.621 | −16.344 | −20.653 | 0.00 | 0.00 | 0.053 | 0.00 | 0.00 |
| ATOM | 857 | HB2 | ASP | 377 | −10.021 | −16.321 | −19.858 | 0.00 | 0.00 | 0.053 | 0.00 | 0.00 |
| ATOM | 858 | CG | ASP | 377 | −10.005 | −17.081 | −21.876 | 0.00 | 0.00 | 0.297 | 9.82 | 4.00 |
| ATOM | 859 | OD1 | ASP | 377 | −9.405 | −16.560 | −22.854 | 0.00 | 0.00 | −0.534 | 8.17 | −18.95 |
| ATOM | 860 | OD2 | ASP | 377 | −10.174 | −18.320 | −21.735 | 0.00 | 0.00 | −0.534 | 8.17 | −18.95 |
| ATOM | 861 | N | SER | 378 | −12.490 | −13.808 | −21.990 | 0.00 | 0.00 | −0.650 | 9.00 | −17.40 |
| ATOM | 862 | HN | SER | 378 | −12.653 | −13.542 | −21.008 | 0.00 | 0.00 | 0.439 | 0.00 | 0.00 |
| ATOM | 863 | CA | SER | 378 | −13.494 | −13.523 | −22.976 | 0.00 | 0.00 | 0.158 | 9.40 | 4.00 |
| ATOM | 864 | HA | SER | 378 | −13.891 | −14.475 | −23.329 | 0.00 | 0.00 | 0.053 | 0.00 | 0.00 |
| ATOM | 865 | C | SER | 378 | −12.838 | −12.766 | −24.086 | 0.00 | 0.00 | 0.396 | 9.82 | 4.00 |
| ATOM | 866 | O | SER | 378 | −13.025 | −13.098 | −25.256 | 0.00 | 0.00 | −0.396 | 8.17 | −17.40 |
| ATOM | 867 | CB | SER | 378 | −14.634 | −12.647 | −22.428 | 0.00 | 0.00 | 0.007 | 12.77 | 4.00 |
| ATOM | 868 | HB1 | SER | 378 | −14.238 | −11.694 | −22.078 | 0.00 | 0.00 | 0.053 | 0.00 | 0.00 |
| ATOM | 869 | HB2 | SER | 378 | −15.126 | −13.151 | −21.596 | 0.00 | 0.00 | 0.053 | 0.00 | 0.00 |
| ATOM | 870 | OG | SER | 378 | −15.591 | −12.400 | −23.447 | 0.00 | 0.00 | −0.537 | 11.04 | −17.40 |
| ATOM | 871 | HG | SER | 378 | −15.654 | −11.387 | −23.621 | 0.00 | 0.00 | 0.424 | 0.00 | 0.00 |
| ATOM | 872 | N | PHE | 379 | −12.032 | −11.739 | −23.760 | 0.00 | 0.00 | −0.650 | 9.00 | −17.40 |
| ATOM | 873 | HN | PHE | 379 | −11.924 | −11.433 | −22.782 | 0.00 | 0.00 | 0.439 | 0.00 | 0.00 |
| ATOM | 874 | CA | PHE | 379 | −11.331 | −11.088 | −24.825 | 0.00 | 0.00 | 0.158 | 9.40 | 4.00 |
| ATOM | 875 | HA | PHE | 379 | −11.072 | −11.857 | −25.553 | 0.00 | 0.00 | 0.053 | 0.00 | 0.00 |
| ATOM | 876 | C | PHE | 379 | −10.117 | −10.458 | −24.228 | 0.00 | 0.00 | 0.396 | 9.82 | 4.00 |
| ATOM | 877 | O | PHE | 379 | −10.180 | −9.902 | −23.133 | 0.00 | 0.00 | −0.396 | 8.17 | −17.40 |
| ATOM | 878 | CB | PHE | 379 | −12.134 | −9.973 | −25.521 | 0.00 | 0.00 | −0.106 | 12.77 | 4.00 |
| ATOM | 879 | HB1 | PHE | 379 | −13.111 | −10.381 | −25.779 | 0.00 | 0.00 | 0.053 | 0.00 | 0.00 |
| ATOM | 880 | HB2 | PHE | 379 | −11.582 | −9.676 | −26.413 | 0.00 | 0.00 | 0.053 | 0.00 | 0.00 |
| ATOM | 881 | CG | PHE | 379 | −12.258 | −8.842 | −24.563 | 0.00 | 0.00 | 0.000 | 7.26 | 0.60 |
| ATOM | 882 | CD1 | PHE | 379 | −13.220 | −8.841 | −23.580 | 0.00 | 0.00 | −0.127 | 10.80 | 0.60 |
| ATOM | 883 | HD1 | PHE | 379 | −13.910 | −9.680 | −23.495 | 0.00 | 0.00 | 0.127 | 0.00 | 0.00 |
| ATOM | 884 | CD2 | PHE | 379 | −11.393 | −7.776 | −24.656 | 0.00 | 0.00 | −0.127 | 10.80 | 0.60 |
| ATOM | 885 | HD2 | PHE | 379 | −10.627 | −7.771 | −25.432 | 0.00 | 0.00 | 0.127 | 0.00 | 0.00 |
| ATOM | 886 | CE1 | PHE | 379 | −13.314 | −7.785 | −22.704 | 0.00 | 0.00 | −0.127 | 10.80 | 0.60 |
| ATOM | 887 | HE1 | PHE | 379 | −14.079 | −7.788 | −21.928 | 0.00 | 0.00 | 0.127 | 0.00 | 0.00 |
| ATOM | 888 | CE2 | PHE | 379 | −11.481 | −6.719 | −23.785 | 0.00 | 0.00 | −0.127 | 10.80 | 0.60 |
| ATOM | 889 | HE2 | PHE | 379 | −10.791 | −5.880 | −23.869 | 0.00 | 0.00 | 0.127 | 0.00 | 0.00 |
| ATOM | 890 | CZ | PHE | 379 | −12.444 | −6.726 | −22.807 | 0.00 | 0.00 | −0.127 | 10.80 | 0.60 |
| ATOM | 891 | HZ | PHE | 379 | −12.519 | −5.890 | −22.111 | 0.00 | 0.00 | 0.127 | 0.00 | 0.00 |
| ATOM | 892 | N | GLY | 380 | −8.962 | −10.556 | −24.920 | 0.00 | 0.00 | −0.650 | 9.00 | −17.40 |
| ATOM | 893 | HN | GLY | 380 | −8.915 | −11.094 | −25.797 | 0.00 | 0.00 | 0.439 | 0.00 | 0.00 |
| ATOM | 894 | CA | GLY | 380 | −7.802 | −9.890 | −24.400 | 0.00 | 0.00 | 0.105 | 9.40 | 4.00 |
| ATOM | 895 | HA1 | GLY | 380 | −7.968 | −9.571 | −23.371 | 0.00 | 0.00 | 0.053 | 0.00 | 0.00 |
| ATOM | 896 | HA2 | GLY | 380 | −7.563 | −9.008 | −24.994 | 0.00 | 0.00 | 0.053 | 0.00 | 0.00 |
| ATOM | 897 | C | GLY | 380 | −6.609 | −10.803 | −24.419 | 0.00 | 0.00 | 0.396 | 9.82 | 4.00 |
| ATOM | 898 | O | GLY | 380 | −6.712 | −11.959 | −24.827 | 0.00 | 0.00 | −0.396 | 8.17 | −17.40 |
| ATOM | 899 | N | THR | 381 | −5.433 | −10.282 | −23.978 | 0.00 | 0.00 | −0.650 | 9.00 | −17.40 |
| ATOM | 900 | HN | THR | 381 | −5.395 | −9.298 | −23.676 | 0.00 | 0.00 | 0.439 | 0.00 | 0.00 |
| ATOM | 901 | CA | THR | 381 | −4.240 | −11.088 | −23.927 | 0.00 | 0.00 | 0.158 | 9.40 | 4.00 |
| ATOM | 902 | HA | THR | 381 | −4.408 | −11.947 | −24.577 | 0.00 | 0.00 | 0.053 | 0.00 | 0.00 |
| ATOM | 903 | C | THR | 381 | −4.050 | −11.498 | −22.501 | 0.00 | 0.00 | 0.396 | 9.82 | 4.00 |
| ATOM | 904 | O | THR | 381 | −3.900 | −10.645 | −21.626 | 0.00 | 0.00 | −0.396 | 8.17 | −17.40 |
| ATOM | 905 | CB | THR | 381 | −3.000 | −10.356 | −24.350 | 0.00 | 0.00 | 0.060 | 9.40 | 4.00 |
| ATOM | 906 | HB | THR | 381 | −2.857 | −9.503 | −23.687 | 0.00 | 0.00 | 0.053 | 0.00 | 0.00 |
| ATOM | 907 | OG1 | THR | 381 | −3.140 | −9.889 | −25.685 | 0.00 | 0.00 | −0.537 | 11.04 | −17.40 |
| ATOM | 908 | HG1 | THR | 381 | −2.855 | −8.901 | −25.735 | 0.00 | 0.00 | 0.424 | 0.00 | 0.00 |
| ATOM | 909 | CG2 | THR | 381 | −1.805 | −11.318 | −24.249 | 0.00 | 0.00 | −0.159 | 16.15 | 4.00 |
| ATOM | 910 | HG21 | THR | 381 | −0.895 | −10.801 | −24.553 | 0.00 | 0.00 | 0.053 | 0.00 | 0.00 |
| ATOM | 911 | HG22 | THR | 381 | −1.700 | −11.661 | −23.220 | 0.00 | 0.00 | 0.053 | 0.00 | 0.00 |
| ATOM | 912 | HG23 | THR | 381 | −1.971 | −12.175 | −24.902 | 0.00 | 0.00 | 0.053 | 0.00 | 0.00 |
| ATOM | 913 | N | GLU | 382 | −4.053 | −12.821 | −22.219 | 0.00 | 0.00 | −0.650 | 9.00 | −17.40 |
| ATOM | 914 | HN | GLU | 382 | −4.125 | −13.529 | −22.964 | 0.00 | 0.00 | 0.439 | 0.00 | 0.00 |
| ATOM | 915 | CA | GLU | 382 | −3.951 | −13.197 | −20.834 | 0.00 | 0.00 | 0.158 | 9.40 | 4.00 |
| ATOM | 916 | HA | GLU | 382 | −3.581 | −12.365 | −20.235 | 0.00 | 0.00 | 0.053 | 0.00 | 0.00 |
| ATOM | 917 | C | GLU | 382 | −3.007 | −14.352 | −20.665 | 0.00 | 0.00 | 0.396 | 9.82 | 4.00 |
| ATOM | 918 | O | GLU | 382 | −3.421 | −15.503 | −20.789 | 0.00 | 0.00 | −0.396 | 8.17 | −17.40 |
| ATOM | 919 | CB | GLU | 382 | −5.278 | −13.718 | −20.255 | 0.00 | 0.00 | −0.106 | 12.77 | 4.00 |
| ATOM | 920 | HB1 | GLU | 382 | −5.159 | −13.834 | −19.178 | 0.00 | 0.00 | 0.053 | 0.00 | 0.00 |
| ATOM | 921 | HB2 | GLU | 382 | −5.502 | −14.677 | −20.722 | 0.00 | 0.00 | 0.053 | 0.00 | 0.00 |
| ATOM | 922 | CG | GLU | 382 | −6.473 | −12.797 | −20.492 | 0.00 | 0.00 | −0.336 | 12.77 | 4.00 |
| ATOM | 923 | HG1 | GLU | 382 | −6.087 | −11.779 | −20.431 | 0.00 | 0.00 | 0.053 | 0.00 | 0.00 |
| ATOM | 924 | HG2 | GLU | 382 | −7.189 | −13.022 | −19.702 | 0.00 | 0.00 | 0.053 | 0.00 | 0.00 |
| ATOM | 925 | CD | GLU | 382 | −7.003 | −13.141 | −21.879 | 0.00 | 0.00 | 0.297 | 9.82 | 4.00 |
| ATOM | 926 | OE1 | GLU | 382 | −6.405 | −14.042 | −22.526 | 0.00 | 0.00 | −0.534 | 8.17 | −18.95 |
| ATOM | 927 | OE2 | GLU | 382 | −8.010 | −12.520 | −22.310 | 0.00 | 0.00 | −0.534 | 8.17 | −18.95 |
| ATOM | 928 | N | PRO | 383 | −1.752 | −14.107 | −20.406 | 0.00 | 0.00 | −0.422 | 9.00 | −17.40 |
| ATOM | 929 | CA | PRO | 383 | −0.921 | −15.252 | −20.115 | 0.00 | 0.00 | 0.158 | 9.40 | 4.00 |
| ATOM | 930 | HA | PRO | 383 | −1.232 | −16.155 | −20.640 | 0.00 | 0.00 | 0.053 | 0.00 | 0.00 |
| ATOM | 931 | CD | PRO | 383 | −1.071 | −13.197 | −21.314 | 0.00 | 0.00 | 0.105 | 12.77 | 4.00 |
| ATOM | 932 | HD1 | PRO | 383 | −1.343 | −12.199 | −20.971 | 0.00 | 0.00 | 0.053 | 0.00 | 0.00 |
| ATOM | 933 | HD2 | PRO | 383 | −1.461 | −13.438 | −22.303 | 0.00 | 0.00 | 0.053 | 0.00 | 0.00 |
| ATOM | 934 | C | PRO | 383 | −0.908 | −15.623 | −18.655 | 0.00 | 0.00 | 0.396 | 9.82 | 4.00 |
| ATOM | 935 | O | PRO | 383 | −1.325 | −14.810 | −17.832 | 0.00 | 0.00 | −0.396 | 8.17 | −17.40 |
| ATOM | 936 | CB | PRO | 383 | 0.448 | −14.994 | −20.749 | 0.00 | 0.00 | −0.106 | 12.77 | 4.00 |
| ATOM | 937 | HB1 | PRO | 383 | 0.496 | −15.678 | −21.596 | 0.00 | 0.00 | 0.053 | 0.00 | 0.00 |
| ATOM | 938 | HB2 | PRO | 383 | 1.167 | −15.221 | −19.962 | 0.00 | 0.00 | 0.053 | 0.00 | 0.00 |
| ATOM | 939 | CG | PRO | 383 | 0.421 | −13.514 | −21.152 | 0.00 | 0.00 | −0.106 | 12.77 | 4.00 |
| ATOM | 940 | HG1 | PRO | 383 | 0.989 | −13.481 | −22.082 | 0.00 | 0.00 | 0.053 | 0.00 | 0.00 |
| ATOM | 941 | HG2 | PRO | 383 | 0.899 | −13.002 | −20.317 | 0.00 | 0.00 | 0.053 | 0.00 | 0.00 |
| ATOM | 942 | N | GLU | 384 | −0.449 | −16.856 | −18.323 | 0.00 | 0.00 | −0.650 | 9.00 | −17.40 |
| ATOM | 943 | HN | GLU | 384 | −0.120 | −17.477 | −19.076 | 0.00 | 0.00 | 0.439 | 0.00 | 0.00 |
| ATOM | 944 | CA | GLU | 384 | −0.400 | −17.338 | −16.961 | 0.00 | 0.00 | 0.158 | 9.40 | 4.00 |
| ATOM | 945 | HA | GLU | 384 | −0.368 | −16.475 | −16.296 | 0.00 | 0.00 | 0.053 | 0.00 | 0.00 |
| ATOM | 946 | C | GLU | 384 | 0.841 | −18.179 | −16.805 | 0.00 | 0.00 | 0.396 | 9.82 | 4.00 |
| ATOM | 947 | O | GLU | 384 | 1.652 | −18.264 | −17.725 | 0.00 | 0.00 | −0.396 | 8.17 | −17.40 |
| ATOM | 948 | CB | GLU | 384 | −1.596 | −18.226 | −16.577 | 0.00 | 0.00 | −0.106 | 12.77 | 4.00 |
| ATOM | 949 | HB1 | GLU | 384 | −1.501 | −18.677 | −15.589 | 0.00 | 0.00 | 0.053 | 0.00 | 0.00 |
| ATOM | 950 | HB2 | GLU | 384 | −1.752 | −19.061 | −17.260 | 0.00 | 0.00 | 0.053 | 0.00 | 0.00 |
| ATOM | 951 | CG | GLU | 384 | −2.928 | −17.474 | −16.550 | 0.00 | 0.00 | −0.336 | 12.77 | 4.00 |
| ATOM | 952 | HG1 | GLU | 384 | −3.107 | −17.073 | −17.548 | 0.00 | 0.00 | 0.053 | 0.00 | 0.00 |
| ATOM | 953 | HG2 | GLU | 384 | −2.846 | −16.672 | −15.816 | 0.00 | 0.00 | 0.053 | 0.00 | 0.00 |
| ATOM | 954 | CD | GLU | 384 | −4.016 | −18.462 | −16.157 | 0.00 | 0.00 | 0.297 | 9.82 | 4.00 |
| ATOM | 955 | OE1 | GLU | 384 | −3.681 | −19.661 | −15.953 | 0.00 | 0.00 | −0.534 | 8.17 | −18.95 |
| ATOM | 956 | OE2 | GLU | 384 | −5.197 | −18.034 | −16.053 | 0.00 | 0.00 | −0.534 | 8.17 | −18.95 |
| ATOM | 957 | N | PHE | 385 | 1.040 | −18.809 | −15.618 | 0.00 | 0.00 | −0.650 | 9.00 | −17.40 |
| ATOM | 958 | HN | PHE | 385 | 0.341 | −18.719 | −14.867 | 0.00 | 0.00 | 0.439 | 0.00 | 0.00 |
| ATOM | 959 | CA | PHE | 385 | 2.223 | −19.603 | −15.402 | 0.00 | 0.00 | 0.158 | 9.40 | 4.00 |
| ATOM | 960 | HA | PHE | 385 | 2.582 | −20.017 | −16.344 | 0.00 | 0.00 | 0.053 | 0.00 | 0.00 |
| ATOM | 961 | C | PHE | 385 | 1.930 | −20.738 | −14.467 | 0.00 | 0.00 | 0.396 | 9.82 | 4.00 |
| ATOM | 962 | O | PHE | 385 | 0.823 | −21.274 | −14.437 | 0.00 | 0.00 | −0.396 | 8.17 | −17.40 |
| ATOM | 963 | CB | PHE | 385 | 3.384 | −18.783 | −14.803 | 0.00 | 0.00 | −0.106 | 12.77 | 4.00 |
| ATOM | 964 | HB1 | PHE | 385 | 3.617 | −17.962 | −15.481 | 0.00 | 0.00 | 0.053 | 0.00 | 0.00 |
| ATOM | 965 | HB2 | PHE | 385 | 4.250 | −19.436 | −14.689 | 0.00 | 0.00 | 0.053 | 0.00 | 0.00 |
| ATOM | 966 | CG | PHE | 385 | 2.958 | −18.252 | −13.474 | 0.00 | 0.00 | 0.000 | 7.26 | 0.60 |
| ATOM | 967 | CD1 | PHE | 385 | 2.027 | −17.241 | −13.397 | 0.00 | 0.00 | −0.127 | 10.80 | 0.60 |
| ATOM | 968 | HD1 | PHE | 385 | 1.598 | −16.832 | −14.312 | 0.00 | 0.00 | 0.127 | 0.00 | 0.00 |
| ATOM | 969 | CD2 | PHE | 385 | 3.505 | −18.737 | −12.307 | 0.00 | 0.00 | −0.127 | 10.80 | 0.60 |
| ATOM | 970 | HD2 | PHE | 385 | 4.258 | −19.524 | −12.352 | 0.00 | 0.00 | 0.127 | 0.00 | 0.00 |
| ATOM | 971 | CE1 | PHE | 385 | 1.632 | −16.741 | −12.178 | 0.00 | 0.00 | −0.127 | 10.80 | 0.60 |
| ATOM | 972 | HE1 | PHE | 385 | 0.888 | −15.946 | −12.132 | 0.00 | 0.00 | 0.127 | 0.00 | 0.00 |
| ATOM | 973 | CE2 | PHE | 385 | 3.114 | −18.241 | −11.085 | 0.00 | 0.00 | −0.127 | 10.80 | 0.60 |
| ATOM | 974 | HE2 | PHE | 385 | 3.550 | −18.640 | −10.169 | 0.00 | 0.00 | 0.127 | 0.00 | 0.00 |
| ATOM | 975 | CZ | PHE | 385 | 2.173 | −17.242 | −11.019 | 0.00 | 0.00 | −0.127 | 10.80 | 0.60 |
| ATOM | 976 | HZ | PHE | 385 | 1.858 | −16.849 | −10.052 | 0.00 | 0.00 | 0.127 | 0.00 | 0.00 |
| ATOM | 977 | N | ASN | 386 | 2.957 | −21.137 | −13.684 | 0.00 | 0.00 | −0.650 | 9.00 | −17.40 |
| ATOM | 978 | HN | ASN | 386 | 3.842 | −20.613 | −13.741 | 0.00 | 0.00 | 0.439 | 0.00 | 0.00 |
| ATOM | 979 | CA | ASN | 386 | 2.887 | −22.250 | −12.777 | 0.00 | 0.00 | 0.158 | 9.40 | 4.00 |
| ATOM | 980 | HA | ASN | 386 | 2.470 | −23.114 | −13.295 | 0.00 | 0.00 | 0.053 | 0.00 | 0.00 |
| ATOM | 981 | C | ASN | 386 | 2.013 | −21.893 | −11.618 | 0.00 | 0.00 | 0.396 | 9.82 | 4.00 |
| ATOM | 982 | O | ASN | 386 | 1.943 | −20.738 | −11.200 | 0.00 | 0.00 | −0.396 | 8.17 | −17.40 |
| ATOM | 983 | CB | ASN | 386 | 4.265 | −22.651 | −12.217 | 0.00 | 0.00 | −0.106 | 12.77 | 4.00 |
| ATOM | 984 | HB1 | ASN | 386 | 4.564 | −21.933 | −11.453 | 0.00 | 0.00 | 0.053 | 0.00 | 0.00 |
| ATOM | 985 | HB2 | ASN | 386 | 4.994 | −22.652 | −13.027 | 0.00 | 0.00 | 0.053 | 0.00 | 0.00 |
| ATOM | 986 | CG | ASN | 386 | 4.171 | −24.043 | −11.607 | 0.00 | 0.00 | 0.396 | 9.82 | 4.00 |
| ATOM | 987 | OD1 | ASN | 386 | 3.132 | −24.698 | −11.660 | 0.00 | 0.00 | −0.396 | 8.17 | −17.40 |
| ATOM | 988 | ND2 | ASN | 386 | 5.298 | −24.514 | −11.008 | 0.00 | 0.00 | −0.879 | 13.25 | −17.40 |
| ATOM | 989 | HD21 | ASN | 386 | 6.149 | −23.935 | −10.984 | 0.00 | 0.00 | 0.439 | 0.00 | 0.00 |
| ATOM | 990 | HD22 | ASN | 386 | 5.300 | −25.450 | −10.578 | 0.00 | 0.00 | 0.439 | 0.00 | 0.00 |
| ATOM | 991 | N | HIS | 387 | 1.313 | −22.906 | −11.068 | 0.00 | 0.00 | −0.650 | 9.00 | −17.40 |
| ATOM | 992 | HN | HIS | 387 | 1.417 | −23.856 | −11.452 | 0.00 | 0.00 | 0.439 | 0.00 | 0.00 |
| ATOM | 993 | CA | HIS | 387 | 0.429 | −22.691 | −9.962 | 0.00 | 0.00 | 0.158 | 9.40 | 4.00 |
| ATOM | 994 | HA | HIS | 387 | 0.374 | −21.615 | −9.798 | 0.00 | 0.00 | 0.053 | 0.00 | 0.00 |
| ATOM | 995 | C | HIS | 387 | 1.015 | −23.399 | −8.785 | 0.00 | 0.00 | 0.396 | 9.82 | 4.00 |
| ATOM | 996 | O | HIS | 387 | 0.328 | −23.638 | −7.792 | 0.00 | 0.00 | −0.396 | 8.17 | −17.40 |
| ATOM | 997 | CB | HIS | 387 | −0.955 | −23.323 | −10.190 | 0.00 | 0.00 | −0.106 | 12.77 | 4.00 |
| ATOM | 998 | HB1 | HIS | 387 | −1.649 | −23.147 | −9.368 | 0.00 | 0.00 | 0.053 | 0.00 | 0.00 |
| ATOM | 999 | HB2 | HIS | 387 | −0.918 | −24.405 | −10.313 | 0.00 | 0.00 | 0.053 | 0.00 | 0.00 |
| ATOM | 1000 | CG | HIS | 387 | −1.647 | −22.805 | −11.414 | 0.00 | 0.00 | 0.241 | 7.26 | 0.60 |
| ATOM | 1001 | ND1 | HIS | 387 | −1.455 | −23.316 | −12.678 | 0.00 | 0.00 | −0.481 | 9.25 | −17.40 |
| ATOM | 1002 | CD2 | HIS | 387 | −2.550 | −21.796 | −11.555 | 0.00 | 0.00 | −0.177 | 10.80 | 0.60 |
| ATOM | 1003 | HD2 | HIS | 387 | −2.923 | −21.178 | −10.738 | 0.00 | 0.00 | 0.127 | 0.00 | 0.00 |
| ATOM | 1004 | CE1 | HIS | 387 | −2.245 | −22.598 | −13.516 | 0.00 | 0.00 | 0.064 | 10.80 | 0.60 |
| ATOM | 1005 | HE1 | HIS | 387 | −2.308 | −22.772 | −14.590 | 0.00 | 0.00 | 0.127 | 0.00 | 0.00 |
| ATOM | 1006 | NE2 | HIS | 387 | −2.928 | −21.664 | −12.879 | 0.00 | 0.00 | −0.292 | 9.25 | −17.40 |
| ATOM | 1007 | HE2 | HIS | 387 | −3.594 | −20.988 | −13.279 | 0.00 | 0.00 | 0.393 | 0.00 | 0.00 |
| ATOM | 1008 | N | ALA | 388 | 2.315 | −23.737 | −8.857 | 0.00 | 0.00 | −0.650 | 9.00 | −17.40 |
| ATOM | 1009 | HN | ALA | 388 | 2.872 | −23.466 | −9.680 | 0.00 | 0.00 | 0.439 | 0.00 | 0.00 |
| ATOM | 1010 | CA | ALA | 388 | 2.926 | −24.473 | −7.791 | 0.00 | 0.00 | 0.158 | 9.40 | 4.00 |
| ATOM | 1011 | HA | ALA | 388 | 2.309 | −25.342 | −7.564 | 0.00 | 0.00 | 0.053 | 0.00 | 0.00 |
| ATOM | 1012 | C | ALA | 388 | 3.029 | −23.591 | −6.593 | 0.00 | 0.00 | 0.396 | 9.82 | 4.00 |
| ATOM | 1013 | O | ALA | 388 | 3.122 | −22.369 | −6.701 | 0.00 | 0.00 | −0.396 | 8.17 | −17.40 |
| ATOM | 1014 | CB | ALA | 388 | 4.343 | −24.975 | −8.121 | 0.00 | 0.00 | −0.159 | 16.15 | 4.00 |
| ATOM | 1015 | HB1 | ALA | 388 | 4.743 | −25.523 | −7.268 | 0.00 | 0.00 | 0.053 | 0.00 | 0.00 |
| ATOM | 1016 | HB2 | ALA | 388 | 4.303 | −25.633 | −8.989 | 0.00 | 0.00 | 0.053 | 0.00 | 0.00 |
| ATOM | 1017 | HB3 | ALA | 388 | 4.988 | −24.124 | −8.340 | 0.00 | 0.00 | 0.053 | 0.00 | 0.00 |
| ATOM | 1018 | N | ASN | 389 | 2.990 | −24.213 | −5.400 | 0.00 | 0.00 | −0.650 | 9.00 | −17.40 |
| ATOM | 1019 | HN | ASN | 389 | 2.878 | −25.236 | −5.371 | 0.00 | 0.00 | 0.439 | 0.00 | 0.00 |
| ATOM | 1020 | CA | ASN | 389 | 3.100 | −23.487 | −4.171 | 0.00 | 0.00 | 0.158 | 9.40 | 4.00 |
| ATOM | 1021 | HA | ASN | 389 | 2.525 | −22.570 | −4.301 | 0.00 | 0.00 | 0.053 | 0.00 | 0.00 |
| ATOM | 1022 | C | ASN | 389 | 4.554 | −23.220 | −3.966 | 0.00 | 0.00 | 0.396 | 9.82 | 4.00 |
| ATOM | 1023 | O | ASN | 389 | 5.400 | −23.911 | −4.531 | 0.00 | 0.00 | −0.396 | 8.17 | −17.40 |
| ATOM | 1024 | CB | ASN | 389 | 2.599 | −24.285 | −2.956 | 0.00 | 0.00 | −0.106 | 12.77 | 4.00 |
| ATOM | 1025 | HB1 | ASN | 389 | 2.832 | −23.716 | −2.056 | 0.00 | 0.00 | 0.053 | 0.00 | 0.00 |
| ATOM | 1026 | HB2 | ASN | 389 | 3.110 | −25.248 | −2.948 | 0.00 | 0.00 | 0.053 | 0.00 | 0.00 |
| ATOM | 1027 | CG | ASN | 389 | 1.096 | −24.474 | −3.100 | 0.00 | 0.00 | 0.396 | 9.82 | 4.00 |
| ATOM | 1028 | OD1 | ASN | 389 | 0.359 | −23.521 | −3.347 | 0.00 | 0.00 | −0.396 | 8.17 | −17.40 |
| ATOM | 1029 | ND2 | ASN | 389 | 0.626 | −25.742 | −2.951 | 0.00 | 0.00 | −0.879 | 13.25 | −17.40 |
| ATOM | 1030 | HD21 | ASN | 389 | 1.278 | −26.512 | −2.744 | 0.00 | 0.00 | 0.439 | 0.00 | 0.00 |
| ATOM | 1031 | HD22 | ASN | 389 | −0.382 | −25.932 | −3.045 | 0.00 | 0.00 | 0.439 | 0.00 | 0.00 |
| ATOM | 1032 | N | TYR | 390 | 4.895 | −22.191 | −3.165 | 0.00 | 0.00 | −0.650 | 9.00 | −17.40 |
| ATOM | 1033 | HN | TYR | 390 | 4.179 | −21.605 | −2.713 | 0.00 | 0.00 | 0.439 | 0.00 | 0.00 |
| ATOM | 1034 | CA | TYR | 390 | 6.292 | −21.944 | −2.966 | 0.00 | 0.00 | 0.158 | 9.40 | 4.00 |
| ATOM | 1035 | HA | TYR | 390 | 6.856 | −22.632 | −3.596 | 0.00 | 0.00 | 0.053 | 0.00 | 0.00 |
| ATOM | 1036 | C | TYR | 390 | 6.609 | −22.172 | −1.528 | 0.00 | 0.00 | 0.396 | 9.82 | 4.00 |
| ATOM | 1037 | O | TYR | 390 | 5.781 | −21.944 | −0.644 | 0.00 | 0.00 | −0.396 | 8.17 | −17.40 |
| ATOM | 1038 | CB | TYR | 390 | 6.764 | −20.527 | −3.348 | 0.00 | 0.00 | −0.106 | 12.77 | 4.00 |
| ATOM | 1039 | HB1 | TYR | 390 | 6.420 | −20.288 | −4.354 | 0.00 | 0.00 | 0.053 | 0.00 | 0.00 |
| ATOM | 1040 | HB2 | TYR | 390 | 7.853 | −20.486 | −3.317 | 0.00 | 0.00 | 0.053 | 0.00 | 0.00 |
| ATOM | 1041 | CG | TYR | 390 | 6.203 | −19.540 | −2.385 | 0.00 | 0.00 | 0.000 | 7.26 | 0.60 |
| ATOM | 1042 | CD1 | TYR | 390 | 6.863 | −19.254 | −1.210 | 0.00 | 0.00 | −0.127 | 10.80 | 0.60 |
| ATOM | 1043 | HD1 | TYR | 390 | 7.803 | −19.758 | −0.985 | 0.00 | 0.00 | 0.127 | 0.00 | 0.00 |
| ATOM | 1044 | CD2 | TYR | 390 | 5.022 | −18.893 | −2.660 | 0.00 | 0.00 | −0.127 | 10.80 | 0.60 |
| ATOM | 1045 | HD2 | TYR | 390 | 4.491 | −19.105 | −3.588 | 0.00 | 0.00 | 0.127 | 0.00 | 0.00 |
| ATOM | 1046 | CE1 | TYR | 390 | 6.353 | −18.343 | −0.318 | 0.00 | 0.00 | −0.127 | 10.80 | 0.60 |
| ATOM | 1047 | HE1 | TYR | 390 | 6.884 | −18.128 | 0.609 | 0.00 | 0.00 | 0.127 | 0.00 | 0.00 |
| ATOM | 1048 | CE2 | TYR | 390 | 4.508 | −17.981 | −1.772 | 0.00 | 0.00 | −0.127 | 10.80 | 0.60 |
| ATOM | 1049 | HE2 | TYR | 390 | 3.570 | −17.474 | −1.997 | 0.00 | 0.00 | 0.127 | 0.00 | 0.00 |
| ATOM | 1050 | CZ | TYR | 390 | 5.171 | −17.704 | −0.600 | 0.00 | 0.00 | 0.027 | 7.26 | 0.60 |
| ATOM | 1051 | OH | TYR | 390 | 4.640 | −16.767 | 0.311 | 0.00 | 0.00 | −0.451 | 10.94 | −17.40 |
| ATOM | 1052 | HH | TYR | 390 | 5.156 | −15.879 | 0.230 | 0.00 | 0.00 | 0.424 | 0.00 | 0.00 |
| ATOM | 1053 | N | ALA | 391 | 7.841 | −22.643 | −1.267 | 0.00 | 0.00 | −0.650 | 9.00 | −17.40 |
| ATOM | 1054 | HN | ALA | 391 | 8.500 | −22.806 | −2.042 | 0.00 | 0.00 | 0.439 | 0.00 | 0.00 |
| ATOM | 1055 | CA | ALA | 391 | 8.245 | −22.920 | 0.076 | 0.00 | 0.00 | 0.158 | 9.40 | 4.00 |
| ATOM | 1056 | HA | ALA | 391 | 7.483 | −23.554 | 0.529 | 0.00 | 0.00 | 0.053 | 0.00 | 0.00 |
| ATOM | 1057 | C | ALA | 391 | 8.358 | −21.620 | 0.795 | 0.00 | 0.00 | 0.396 | 9.82 | 4.00 |
| ATOM | 1058 | O | ALA | 391 | 8.960 | −20.667 | 0.306 | 0.00 | 0.00 | −0.396 | 8.17 | −17.40 |
| ATOM | 1059 | CB | ALA | 391 | 9.602 | −23.637 | 0.177 | 0.00 | 0.00 | −0.159 | 16.15 | 4.00 |
| ATOM | 1060 | HB1 | ALA | 391 | 9.843 | −23.814 | 1.225 | 0.00 | 0.00 | 0.053 | 0.00 | 0.00 |
| ATOM | 1061 | HB2 | ALA | 391 | 9.550 | −24.590 | −0.349 | 0.00 | 0.00 | 0.053 | 0.00 | 0.00 |
| ATOM | 1062 | HB3 | ALA | 391 | 10.376 | −23.016 | −0.273 | 0.00 | 0.00 | 0.053 | 0.00 | 0.00 |
| ATOM | 1063 | N | GLN | 392 | 7.753 | −21.565 | 1.992 | 0.00 | 0.00 | −0.650 | 9.00 | −17.40 |
| ATOM | 1064 | HN | GLN | 392 | 7.261 | −22.401 | 2.339 | 0.00 | 0.00 | 0.439 | 0.00 | 0.00 |
| ATOM | 1065 | CA | GLN | 392 | 7.767 | −20.387 | 2.803 | 0.00 | 0.00 | 0.158 | 9.40 | 4.00 |
| ATOM | 1066 | HA | GLN | 392 | 8.227 | −19.586 | 2.224 | 0.00 | 0.00 | 0.053 | 0.00 | 0.00 |
| ATOM | 1067 | C | GLN | 392 | 8.562 | −20.683 | 4.030 | 0.00 | 0.00 | 0.396 | 9.82 | 4.00 |
| ATOM | 1068 | O | GLN | 392 | 8.655 | −21.831 | 4.463 | 0.00 | 0.00 | −0.396 | 8.17 | −17.40 |
| ATOM | 1069 | CB | GLN | 392 | 6.343 | −19.936 | 3.186 | 0.00 | 0.00 | −0.106 | 12.77 | 4.00 |
| ATOM | 1070 | HB1 | GLN | 392 | 5.828 | −19.620 | 2.279 | 0.00 | 0.00 | 0.053 | 0.00 | 0.00 |
| ATOM | 1071 | HB2 | GLN | 392 | 6.423 | −19.107 | 3.889 | 0.00 | 0.00 | 0.053 | 0.00 | 0.00 |
| ATOM | 1072 | CG | GLN | 392 | 5.495 | −21.030 | 3.848 | 0.00 | 0.00 | −0.106 | 12.77 | 4.00 |
| ATOM | 1073 | HG1 | GLN | 392 | 5.734 | −22.007 | 3.427 | 0.00 | 0.00 | 0.053 | 0.00 | 0.00 |
| ATOM | 1074 | HG2 | GLN | 392 | 4.433 | −20.839 | 3.691 | 0.00 | 0.00 | 0.053 | 0.00 | 0.00 |
| ATOM | 1075 | CD | GLN | 392 | 5.757 | −21.076 | 5.344 | 0.00 | 0.00 | 0.396 | 9.82 | 4.00 |
| ATOM | 1076 | OE1 | GLN | 392 | 5.387 | −20.157 | 6.073 | 0.00 | 0.00 | −0.396 | 8.17 | −17.40 |
| ATOM | 1077 | NE2 | GLN | 392 | 6.392 | −22.180 | 5.822 | 0.00 | 0.00 | −0.879 | 13.25 | −17.40 |
| ATOM | 1078 | HE21 | GLN | 392 | 6.685 | −22.926 | 5.175 | 0.00 | 0.00 | 0.439 | 0.00 | 0.00 |
| ATOM | 1079 | HE22 | GLN | 392 | 6.580 | −22.270 | 6.831 | 0.00 | 0.00 | 0.439 | 0.00 | 0.00 |
| ATOM | 1060 | N | SER | 393 | 9.191 | −19.641 | 4.606 | 0.00 | 0.00 | −0.650 | 9.00 | −17.40 |
| ATOM | 1081 | HN | SER | 393 | 9.106 | −18.701 | 4.193 | 0.00 | 0.00 | 0.439 | 0.00 | 0.00 |
| ATOM | 1082 | CA | SER | 393 | 9.977 | −19.828 | 5.788 | 0.00 | 0.00 | 0.158 | 9.40 | 4.00 |
| ATOM | 1083 | HA | SER | 393 | 10.663 | −20.650 | 5.583 | 0.00 | 0.00 | 0.053 | 0.00 | 0.00 |
| ATOM | 1084 | C | SER | 393 | 9.026 | −20.155 | 6.890 | 0.00 | 0.00 | 0.396 | 9.82 | 4.00 |
| ATOM | 1085 | O | SER | 393 | 7.821 | −19.956 | 6.763 | 0.00 | 0.00 | −0.396 | 8.17 | −17.40 |
| ATOM | 1086 | CB | SER | 393 | 10.778 | −18.583 | 6.204 | 0.00 | 0.00 | 0.007 | 12.77 | 4.00 |
| ATOM | 1087 | HB1 | SER | 393 | 11.405 | −18.248 | 5.378 | 0.00 | 0.00 | 0.053 | 0.00 | 0.00 |
| ATOM | 1088 | HB2 | SER | 393 | 11.414 | −18.815 | 7.058 | 0.00 | 0.00 | 0.053 | 0.00 | 0.00 |
| ATOM | 1089 | OG | SER | 393 | 9.893 | −17.531 | 6.564 | 0.00 | 0.00 | −0.537 | 11.04 | −17.40 |
| ATOM | 1090 | HG | SER | 393 | 10.398 | −16.633 | 6.539 | 0.00 | 0.00 | 0.424 | 0.00 | 0.00 |
| ATOM | 1091 | N | LYS | 394 | 9.550 | −20.668 | 8.017 | 0.00 | 0.00 | −0.650 | 9.00 | −17.40 |
| ATOM | 1092 | HN | LYS | 394 | 10.571 | −20.771 | 8.106 | 0.00 | 0.00 | 0.439 | 0.00 | 0.00 |
| ATOM | 1093 | CA | LYS | 394 | 8.698 | −21.073 | 9.096 | 0.00 | 0.00 | 0.158 | 9.40 | 4.00 |
| ATOM | 1094 | HA | LYS | 394 | 8.015 | −21.841 | 8.731 | 0.00 | 0.00 | 0.053 | 0.00 | 0.00 |
| ATOM | 1095 | C | LYS | 394 | 7.932 | −19.882 | 9.572 | 0.00 | 0.00 | 0.396 | 9.82 | 4.00 |
| ATOM | 1096 | O | LYS | 394 | 6.758 | −19.987 | 9.923 | 0.00 | 0.00 | −0.396 | 8.17 | −17.40 |
| ATOM | 1097 | CB | LYS | 394 | 9.478 | −21.622 | 10.304 | 0.00 | 0.00 | −0.106 | 12.77 | 4.00 |
| ATOM | 1098 | HB1 | LYS | 394 | 8.772 | −21.803 | 11.115 | 0.00 | 0.00 | 0.053 | 0.00 | 0.00 |
| ATOM | 1099 | HB2 | LYS | 394 | 10.219 | −20.880 | 10.600 | 0.00 | 0.00 | 0.053 | 0.00 | 0.00 |
| ATOM | 1100 | CG | LYS | 394 | 10.217 | −22.934 | 10.028 | 0.00 | 0.00 | −0.106 | 12.77 | 4.00 |
| ATOM | 1101 | HG1 | LYS | 394 | 9.513 | −23.639 | 9.586 | 0.00 | 0.00 | 0.053 | 0.00 | 0.00 |
| ATOM | 1102 | HG2 | LYS | 394 | 10.600 | −23.319 | 10.973 | 0.00 | 0.00 | 0.053 | 0.00 | 0.00 |
| ATOM | 1103 | CD | LYS | 394 | 11.399 | −22.788 | 9.068 | 0.00 | 0.00 | −0.106 | 12.77 | 4.00 |
| ATOM | 1104 | HD1 | LYS | 394 | 12.165 | −22.100 | 9.424 | 0.00 | 0.00 | 0.053 | 0.00 | 0.00 |
| ATOM | 1105 | HD2 | LYS | 394 | 11.115 | −22.415 | 8.084 | 0.00 | 0.00 | 0.053 | 0.00 | 0.00 |
| ATOM | 1106 | CE | LYS | 394 | 12.134 | −24.103 | 8.802 | 0.00 | 0.00 | 0.099 | 12.77 | 4.00 |
| ATOM | 1107 | HE1 | LYS | 394 | 11.452 | −24.830 | 8.362 | 0.00 | 0.00 | 0.053 | 0.00 | 0.00 |
| ATOM | 1108 | HE2 | LYS | 394 | 12.526 | −24.508 | 9.735 | 0.00 | 0.00 | 0.053 | 0.00 | 0.00 |
| ATOM | 1109 | NZ | LYS | 394 | 13.260 | −23.876 | 7.868 | 0.00 | 0.00 | −0.045 | 13.25 | −39.20 |
| ATOM | 1110 | HZ1 | LYS | 394 | 13.748 | −24.766 | 7.695 | 0.00 | 0.00 | 0.280 | 0.00 | 0.00 |
| ATOM | 1111 | HZ2 | LYS | 394 | 12.899 | −23.505 | 6.978 | 0.00 | 0.00 | 0.280 | 0.00 | 0.00 |
| ATOM | 1112 | HZ3 | LYS | 394 | 13.918 | −23.199 | 8.280 | 0.00 | 0.00 | 0.280 | 0.00 | 0.00 |
| ATOM | 1113 | N | GLY | 395 | 8.594 | −18.713 | 9.603 | 0.00 | 0.00 | −0.650 | 9.00 | −17.40 |
| ATOM | 1114 | HN | GLY | 395 | 9.562 | −18.684 | 9.254 | 0.00 | 0.00 | 0.439 | 0.00 | 0.00 |
| ATOM | 1115 | CA | GLY | 395 | 8.006 | −17.505 | 10.103 | 0.00 | 0.00 | 0.105 | 9.40 | 4.00 |
| ATOM | 1116 | HA1 | GLY | 395 | 8.729 | −16.692 | 10.176 | 0.00 | 0.00 | 0.053 | 0.00 | 0.00 |
| ATOM | 1117 | HA2 | GLY | 395 | 7.582 | −17.632 | 11.099 | 0.00 | 0.00 | 0.053 | 0.00 | 0.00 |
| ATOM | 1118 | C | GLY | 395 | 6.894 | −17.008 | 9.228 | 0.00 | 0.00 | 0.396 | 9.82 | 4.00 |
| ATOM | 1119 | O | GLY | 395 | 5.941 | −16.412 | 9.727 | 0.00 | 0.00 | −0.396 | 8.17 | −17.40 |
| ATOM | 1120 | N | HIS | 396 | 6.979 | −17.215 | 7.899 | 0.00 | 0.00 | −0.650 | 9.00 | −17.40 |
| ATOM | 1121 | HN | HIS | 396 | 7.720 | −17.816 | 7.511 | 0.00 | 0.00 | 0.439 | 0.00 | 0.00 |
| ATOM | 1122 | CA | HIS | 396 | 6.021 | −16.586 | 7.030 | 0.00 | 0.00 | 0.158 | 9.40 | 4.00 |
| ATOM | 1123 | HA | HIS | 396 | 5.541 | −15.723 | 7.491 | 0.00 | 0.00 | 0.053 | 0.00 | 0.00 |
| ATOM | 1124 | C | HIS | 396 | 4.915 | −17.512 | 6.626 | 0.00 | 0.00 | 0.396 | 9.82 | 4.00 |
| ATOM | 1125 | O | HIS | 396 | 4.625 | −17.651 | 5.438 | 0.00 | 0.00 | −0.396 | 8.17 | −17.40 |
| ATOM | 1126 | CB | HIS | 396 | 6.691 | −16.087 | 5.742 | 0.00 | 0.00 | −0.106 | 12.77 | 4.00 |
| ATOM | 1127 | HB1 | HIS | 396 | 5.966 | −15.662 | 5.048 | 0.00 | 0.00 | 0.053 | 0.00 | 0.00 |
| ATOM | 1128 | HB2 | HIS | 396 | 7.205 | −16.891 | 5.216 | 0.00 | 0.00 | 0.053 | 0.00 | 0.00 |
| ATOM | 1129 | CG | HIS | 396 | 7.709 | −15.023 | 6.021 | 0.00 | 0.00 | 0.241 | 7.26 | 0.60 |
| ATOM | 1130 | ND1 | HIS | 396 | 8.698 | −14.645 | 5.143 | 0.00 | 0.00 | −0.481 | 9.25 | −17.40 |
| ATOM | 1131 | CD2 | HIS | 396 | 7.871 | −14.242 | 7.124 | 0.00 | 0.00 | −0.177 | 10.80 | 0.60 |
| ATOM | 1132 | HD2 | HIS | 396 | 7.244 | −14.287 | 8.015 | 0.00 | 0.00 | 0.127 | 0.00 | 0.00 |
| ATOM | 1133 | CE1 | HIS | 396 | 9.406 | −13.661 | 5.752 | 0.00 | 0.00 | 0.064 | 10.80 | 0.60 |
| ATOM | 1134 | HE1 | HIS | 396 | 10.260 | −13.157 | 5.299 | 0.00 | 0.00 | 0.127 | 0.00 | 0.00 |
| ATOM | 1135 | NE2 | HIS | 396 | 8.942 | −13.383 | 6.957 | 0.00 | 0.00 | −0.292 | 9.25 | −17.40 |
| ATOM | 1136 | HE2 | HIS | 396 | 9.298 | −12.683 | 7.623 | 0.00 | 0.00 | 0.393 | 0.00 | 0.00 |
| ATOM | 1137 | N | LYS | 397 | 4.225 | −18.143 | 7.593 | 0.00 | 0.00 | −0.650 | 9.00 | −17.40 |
| ATOM | 1138 | HN | LYS | 397 | 4.457 | −17.990 | 8.585 | 0.00 | 0.00 | 0.439 | 0.00 | 0.00 |
| ATOM | 1139 | CA | LYS | 397 | 3.161 | −19.032 | 7.223 | 0.00 | 0.00 | 0.158 | 9.40 | 4.00 |
| ATOM | 1140 | HA | LYS | 397 | 3.551 | −19.782 | 6.535 | 0.00 | 0.00 | 0.053 | 0.00 | 0.00 |
| ATOM | 1141 | C | LYS | 397 | 2.075 | −18.240 | 6.558 | 0.00 | 0.00 | 0.396 | 9.82 | 4.00 |
| ATOM | 1142 | O | LYS | 397 | 1.586 | −18.609 | 5.491 | 0.00 | 0.00 | −0.396 | 8.17 | −17.40 |
| ATOM | 1143 | CB | LYS | 397 | 2.529 | −19.743 | 8.432 | 0.00 | 0.00 | −0.106 | 12.77 | 4.00 |
| ATOM | 1144 | HB1 | LYS | 397 | 1.649 | −20.336 | 8.182 | 0.00 | 0.00 | 0.053 | 0.00 | 0.00 |
| ATOM | 1145 | HB2 | LYS | 397 | 2.194 | −19.063 | 9.215 | 0.00 | 0.00 | 0.053 | 0.00 | 0.00 |
| ATOM | 1146 | CG | LYS | 397 | 3.474 | −20.719 | 9.136 | 0.00 | 0.00 | −0.106 | 12.77 | 4.00 |
| ATOM | 1147 | HG1 | LYS | 397 | 3.036 | −21.199 | 10.011 | 0.00 | 0.00 | 0.053 | 0.00 | 0.00 |
| ATOM | 1148 | HG2 | LYS | 397 | 4.391 | −20.256 | 9.499 | 0.00 | 0.00 | 0.053 | 0.00 | 0.00 |
| ATOM | 1149 | CD | LYS | 397 | 3.941 | −21.873 | 8.247 | 0.00 | 0.00 | −0.106 | 12.77 | 4.00 |
| ATOM | 1150 | HD1 | LYS | 397 | 4.486 | −21.549 | 7.360 | 0.00 | 0.00 | 0.053 | 0.00 | 0.00 |
| ATOM | 1151 | HD2 | LYS | 397 | 3.126 | −22.490 | 7.868 | 0.00 | 0.00 | 0.053 | 0.00 | 0.00 |
| ATOM | 1152 | CE | LYS | 397 | 4.881 | −22.850 | 8.954 | 0.00 | 0.00 | 0.099 | 12.77 | 4.00 |
| ATOM | 1153 | HE1 | LYS | 397 | 4.381 | −23.292 | 9.816 | 0.00 | 0.00 | 0.053 | 0.00 | 0.00 |
| ATOM | 1154 | HE2 | LYS | 397 | 5.776 | −22.328 | 9.294 | 0.00 | 0.00 | 0.053 | 0.00 | 0.00 |
| ATOM | 1155 | NZ | LYS | 397 | 5.281 | −23.931 | 8.026 | 0.00 | 0.00 | −0.045 | 13.25 | −39.20 |
| ATOM | 1156 | HZ1 | LYS | 397 | 5.914 | −24.584 | 8.511 | 0.00 | 0.00 | 0.280 | 0.00 | 0.00 |
| ATOM | 1157 | HZ2 | LYS | 397 | 4.443 | −24.438 | 7.708 | 0.00 | 0.00 | 0.280 | 0.00 | 0.00 |
| ATOM | 1158 | HZ3 | LYS | 397 | 5.765 | −23.524 | 7.213 | 0.00 | 0.00 | 0.280 | 0.00 | 0.00 |
| ATOM | 1159 | N | THR | 398 | 1.682 | −17.112 | 7.180 | 0.00 | 0.00 | −0.650 | 9.00 | −17.40 |
| ATOM | 1160 | HN | THR | 398 | 2.170 | −16.833 | 8.043 | 0.00 | 0.00 | 0.439 | 0.00 | 0.00 |
| ATOM | 1161 | CA | THR | 398 | 0.614 | −16.279 | 6.698 | 0.00 | 0.00 | 0.158 | 9.40 | 4.00 |
| ATOM | 1162 | HA | THR | 398 | −0.293 | −16.839 | 6.470 | 0.00 | 0.00 | 0.053 | 0.00 | 0.00 |
| ATOM | 1163 | C | THR | 398 | 0.968 | −15.541 | 5.431 | 0.00 | 0.00 | 0.396 | 9.82 | 4.00 |
| ATOM | 1164 | O | THR | 398 | 0.064 | −15.261 | 4.645 | 0.00 | 0.00 | −0.396 | 8.17 | −17.40 |
| ATOM | 1165 | CB | THR | 398 | 0.071 | −15.319 | 7.730 | 0.00 | 0.00 | 0.060 | 9.40 | 4.00 |
| ATOM | 1166 | HB | THR | 398 | −0.725 | −14.737 | 7.265 | 0.00 | 0.00 | 0.053 | 0.00 | 0.00 |
| ATOM | 1167 | OG1 | THR | 398 | 1.064 | −14.416 | 8.190 | 0.00 | 0.00 | −0.537 | 11.04 | −17.40 |
| ATOM | 1168 | HG1 | THR | 398 | 1.923 | −14.537 | 7.634 | 0.00 | 0.00 | 0.424 | 0.00 | 0.00 |
| ATOM | 1169 | CG2 | THR | 398 | −0.471 | −16.143 | 8.909 | 0.00 | 0.00 | −0.159 | 16.15 | 4.00 |
| ATOM | 1170 | HG21 | THR | 398 | −0.870 | −15.471 | 9.669 | 0.00 | 0.00 | 0.053 | 0.00 | 0.00 |
| ATOM | 1171 | HG22 | THR | 398 | −1.263 | −16.804 | 8.557 | 0.00 | 0.00 | 0.053 | 0.00 | 0.00 |
| ATOM | 1172 | HG23 | THR | 398 | 0.335 | −16.739 | 9.338 | 0.00 | 0.00 | 0.053 | 0.00 | 0.00 |
| ATOM | 1173 | N | PRO | 399 | 2.204 | −15.195 | 5.166 | 0.00 | 0.00 | −0.422 | 9.00 | −17.40 |
| ATOM | 1174 | CA | PRO | 399 | 2.481 | −14.452 | 3.959 | 0.00 | 0.00 | 0.158 | 9.40 | 4.00 |
| ATOM | 1175 | HA | PRO | 399 | 1.729 | −13.694 | 3.740 | 0.00 | 0.00 | 0.053 | 0.00 | 0.00 |
| ATOM | 1176 | CD | PRO | 399 | 3.104 | −14.778 | 6.231 | 0.00 | 0.00 | 0.105 | 12.77 | 4.00 |
| ATOM | 1177 | HD1 | PRO | 399 | 3.312 | −15.686 | 6.797 | 0.00 | 0.00 | 0.053 | 0.00 | 0.00 |
| ATOM | 1178 | HD2 | PRO | 399 | 2.546 | −14.031 | 6.796 | 0.00 | 0.00 | 0.053 | 0.00 | 0.00 |
| ATOM | 1179 | C | PRO | 399 | 2.548 | −15.314 | 2.741 | 0.00 | 0.00 | 0.396 | 9.82 | 4.00 |
| ATOM | 1180 | O | PRO | 399 | 3.141 | −14.891 | 1.751 | 0.00 | 0.00 | −0.396 | 8.17 | −17.40 |
| ATOM | 1181 | CB | PRO | 399 | 3.762 | −13.652 | 4.204 | 0.00 | 0.00 | −0.106 | 12.77 | 4.00 |
| ATOM | 1182 | HB1 | PRO | 399 | 3.435 | −12.615 | 4.277 | 0.00 | 0.00 | 0.053 | 0.00 | 0.00 |
| ATOM | 1183 | HB2 | PRO | 399 | 4.388 | −13.853 | 3.335 | 0.00 | 0.00 | 0.053 | 0.00 | 0.00 |
| ATOM | 1184 | CG | PRO | 399 | 4.331 | −14.211 | 5.514 | 0.00 | 0.00 | −0.106 | 12.77 | 4.00 |
| ATOM | 1185 | HG1 | PRO | 399 | 4.810 | −13.425 | 6.098 | 0.00 | 0.00 | 0.053 | 0.00 | 0.00 |
| ATOM | 1186 | HG2 | PRO | 399 | 5.074 | −14.984 | 5.317 | 0.00 | 0.00 | 0.053 | 0.00 | 0.00 |
| ATOM | 1187 | N | TRP | 400 | 1.929 | −16.504 | 2.778 | 0.00 | 0.00 | −0.650 | 9.00 | −17.40 |
| ATOM | 1188 | HN | TRP | 400 | 1.374 | −16.740 | 3.613 | 0.00 | 0.00 | 0.439 | 0.00 | 0.00 |
| ATOM | 1189 | CA | TRP | 400 | 2.001 | −17.463 | 1.715 | 0.00 | 0.00 | 0.158 | 9.40 | 4.00 |
| ATOM | 1190 | HA | TRP | 400 | 3.020 | −17.838 | 1.623 | 0.00 | 0.00 | 0.053 | 0.00 | 0.00 |
| ATOM | 1191 | C | TRP | 400 | 1.596 | −16.821 | 0.421 | 0.00 | 0.00 | 0.396 | 9.82 | 4.00 |
| ATOM | 1192 | O | TRP | 400 | 2.165 | −17.158 | −0.615 | 0.00 | 0.00 | −0.396 | 8.17 | −17.40 |
| ATOM | 1193 | CB | TRP | 400 | 1.064 | −18.657 | 1.962 | 0.00 | 0.00 | −0.106 | 12.77 | 4.00 |
| ATOM | 1194 | HB1 | TRP | 400 | 0.043 | −18.279 | 2.020 | 0.00 | 0.00 | 0.053 | 0.00 | 0.00 |
| ATOM | 1195 | HB2 | TRP | 400 | 1.357 | −19.128 | 2.900 | 0.00 | 0.00 | 0.053 | 0.00 | 0.00 |
| ATOM | 1196 | CG | TRP | 400 | 1.089 | −19.725 | 0.896 | 0.00 | 0.00 | 0.000 | 7.26 | 0.60 |
| ATOM | 1197 | CD1 | TRP | 400 | 0.340 | −19.850 | −0.238 | 0.00 | 0.00 | −0.177 | 10.80 | 0.60 |
| ATOM | 1198 | HD1 | TRP | 400 | −0.419 | −19.141 | −0.569 | 0.00 | 0.00 | 0.127 | 0.00 | 0.00 |
| ATOM | 1199 | CD2 | TRP | 400 | 1.958 | −20.866 | 0.940 | 0.00 | 0.00 | 0.000 | 6.80 | 0.60 |
| ATOM | 1200 | NE1 | TRP | 400 | 0.687 | −21.004 | −0.901 | 0.00 | 0.00 | −0.292 | 9.00 | −17.40 |
| ATOM | 1201 | HE1 | TRP | 400 | 0.273 | −21.339 | −1.783 | 0.00 | 0.00 | 0.393 | 0.00 | 0.00 |
| ATOM | 1202 | CE2 | TRP | 400 | 1.682 | −21.637 | −0.187 | 0.00 | 0.00 | −0.050 | 6.80 | 0.60 |
| ATOM | 1203 | CE3 | TRP | 400 | 2.905 | −21.242 | 1.849 | 0.00 | 0.00 | −0.127 | 10.80 | 0.60 |
| ATOM | 1204 | HE3 | TRP | 400 | 3.116 | −20.638 | 2.731 | 0.00 | 0.00 | 0.127 | 0.00 | 0.00 |
| ATOM | 1205 | CZ2 | TRP | 400 | 2.353 | −22.803 | −0.421 | 0.00 | 0.00 | −0.127 | 10.80 | 0.60 |
| ATOM | 1206 | HZ2 | TRP | 400 | 2.137 | −23.412 | −1.299 | 0.00 | 0.00 | 0.127 | 0.00 | 0.00 |
| ATOM | 1207 | CZ3 | TRP | 400 | 3.583 | −22.416 | 1.606 | 0.00 | 0.00 | −0.127 | 10.80 | 0.60 |
| ATOM | 1208 | HZ3 | TRP | 400 | 4.349 | −22.746 | 2.308 | 0.00 | 0.00 | 0.127 | 0.00 | 0.00 |
| ATOM | 1209 | CH2 | TRP | 400 | 3.312 | −23.182 | 0.492 | 0.00 | 0.00 | −0.127 | 10.80 | 0.60 |
| ATOM | 1210 | HH2 | TRP | 400 | 3.868 | −24.106 | 0.331 | 0.00 | 0.00 | 0.127 | 0.00 | 0.00 |
| ATOM | 1211 | N | GLY | 401 | 0.602 | −15.903 | 0.439 | 0.00 | 0.00 | −0.650 | 9.00 | −17.40 |
| ATOM | 1212 | HN | GLY | 401 | 0.162 | −15.671 | 1.341 | 0.00 | 0.00 | 0.439 | 0.00 | 0.00 |
| ATOM | 1213 | CA | GLY | 401 | 0.131 | −15.234 | −0.751 | 0.00 | 0.00 | 0.105 | 9.40 | 4.00 |
| ATOM | 1214 | HA1 | GLY | 401 | −0.487 | −14.382 | −0.467 | 0.00 | 0.00 | 0.053 | 0.00 | 0.00 |
| ATOM | 1215 | HA2 | GLY | 401 | −0.459 | −15.929 | −1.348 | 0.00 | 0.00 | 0.053 | 0.00 | 0.00 |
| ATOM | 1216 | C | GLY | 401 | 1.306 | −14.757 | −1.553 | 0.00 | 0.00 | 0.396 | 9.82 | 4.00 |
| ATOM | 1217 | O | GLY | 401 | 1.948 | −13.766 | −1.211 | 0.00 | 0.00 | −0.396 | 8.17 | −17.40 |
| ATOM | 1218 | N | LYS | 402 | 1.601 | −15.470 | −2.663 | 0.00 | 0.00 | −0.650 | 9.00 | −17.40 |
| ATOM | 1219 | HN | LYS | 402 | 0.978 | −16.244 | −2.935 | 0.00 | 0.00 | 0.439 | 0.00 | 0.00 |
| ATOM | 1220 | CA | LYS | 402 | 2.750 | −15.189 | −3.476 | 0.00 | 0.00 | 0.158 | 9.40 | 4.00 |
| ATOM | 1221 | HA | LYS | 402 | 3.617 | −14.972 | −2.851 | 0.00 | 0.00 | 0.053 | 0.00 | 0.00 |
| ATOM | 1222 | C | LYS | 402 | 2.495 | −14.010 | −4.352 | 0.00 | 0.00 | 0.396 | 9.82 | 4.00 |
| ATOM | 1223 | O | LYS | 402 | 2.506 | −14.109 | −5.579 | 0.00 | 0.00 | −0.396 | 8.17 | −17.40 |
| ATOM | 1224 | CB | LYS | 402 | 3.170 | −16.381 | −4.352 | 0.00 | 0.00 | −0.106 | 12.77 | 4.00 |
| ATOM | 1225 | HB1 | LYS | 402 | 3.474 | −17.197 | −3.697 | 0.00 | 0.00 | 0.053 | 0.00 | 0.00 |
| ATOM | 1226 | HB2 | LYS | 402 | 4.001 | −16.066 | −4.983 | 0.00 | 0.00 | 0.053 | 0.00 | 0.00 |
| ATOM | 1227 | CG | LYS | 402 | 2.065 | −16.907 | −5.267 | 0.00 | 0.00 | −0.106 | 12.77 | 4.00 |
| ATOM | 1228 | HG1 | LYS | 402 | 1.597 | −16.125 | −5.865 | 0.00 | 0.00 | 0.053 | 0.00 | 0.00 |
| ATOM | 1229 | HG2 | LYS | 402 | 1.252 | −17.387 | −4.723 | 0.00 | 0.00 | 0.053 | 0.00 | 0.00 |
| ATOM | 1230 | CD | LYS | 402 | 2.562 | −17.950 | −6.269 | 0.00 | 0.00 | −0.106 | 12.77 | 4.00 |
| ATOM | 1231 | HD1 | LYS | 402 | 3.126 | −18.764 | −5.813 | 0.00 | 0.00 | 0.053 | 0.00 | 0.00 |
| ATOM | 1232 | HD2 | LYS | 402 | 3.225 | −17.545 | −7.033 | 0.00 | 0.00 | 0.053 | 0.00 | 0.00 |
| ATOM | 1233 | CE | LYS | 402 | 1.442 | −18.639 | −7.049 | 0.00 | 0.00 | 0.099 | 12.77 | 4.00 |
| ATOM | 1234 | HE1 | LYS | 402 | 0.893 | −17.905 | −7.639 | 0.00 | 0.00 | 0.053 | 0.00 | 0.00 |
| ATOM | 1235 | HE2 | LYS | 402 | 0.753 | −19.126 | −6.359 | 0.00 | 0.00 | 0.053 | 0.00 | 0.00 |
| ATOM | 1236 | NZ | LYS | 402 | 2.012 | −19.658 | −7.958 | 0.00 | 0.00 | −0.045 | 13.25 | −39.20 |
| ATOM | 1237 | HZ1 | LYS | 402 | 1.252 | −20.117 | −8.479 | 0.00 | 0.00 | 0.280 | 0.00 | 0.00 |
| ATOM | 1238 | HZ2 | LYS | 402 | 2.657 | −19.205 | −8.621 | 0.00 | 0.00 | 0.280 | 0.00 | 0.00 |
| ATOM | 1239 | HZ3 | LYS | 402 | 2.524 | −20.362 | −7.407 | 0.00 | 0.00 | 0.280 | 0.00 | 0.00 |
| ATOM | 1240 | N | TRP | 403 | 2.261 | −12.848 | −3.720 | 0.00 | 0.00 | −0.650 | 9.00 | −17.40 |
| ATOM | 1241 | HN | TRP | 403 | 2.201 | −12.837 | −2.692 | 0.00 | 0.00 | 0.439 | 0.00 | 0.00 |
| ATOM | 1242 | CA | TRP | 403 | 2.094 | −11.629 | −4.443 | 0.00 | 0.00 | 0.158 | 9.40 | 4.00 |
| ATOM | 1243 | HA | TRP | 403 | 1.332 | −11.826 | −5.197 | 0.00 | 0.00 | 0.053 | 0.00 | 0.00 |
| ATOM | 1244 | C | TRP | 403 | 3.420 | −11.318 | −5.039 | 0.00 | 0.00 | 0.396 | 9.82 | 4.00 |
| ATOM | 1245 | O | TRP | 403 | 3.505 | −10.725 | −6.101 | 0.00 | 0.00 | −0.396 | 8.17 | −17.40 |
| ATOM | 1246 | CB | TRP | 403 | 1.696 | −10.438 | −3.550 | 0.00 | 0.00 | −0.106 | 12.77 | 4.00 |
| ATOM | 1247 | HB1 | TRP | 403 | 0.746 | −10.675 | −3.070 | 0.00 | 0.00 | 0.053 | 0.00 | 0.00 |
| ATOM | 1248 | HB2 | TRP | 403 | 1.599 | −9.556 | −4.183 | 0.00 | 0.00 | 0.053 | 0.00 | 0.00 |
| ATOM | 1249 | CG | TRP | 403 | 2.687 | −10.108 | −2.458 | 0.00 | 0.00 | 0.000 | 7.26 | 0.60 |
| ATOM | 1250 | CD1 | TRP | 403 | 3.861 | −9.417 | −2.536 | 0.00 | 0.00 | −0.177 | 10.80 | 0.60 |
| ATOM | 1251 | HD1 | TRP | 403 | 4.276 | −8.991 | −3.449 | 0.00 | 0.00 | 0.127 | 0.00 | 0.00 |
| ATOM | 1252 | CD2 | TRP | 403 | 2.524 | −10.476 | −1.079 | 0.00 | 0.00 | 0.000 | 6.80 | 0.60 |
| ATOM | 1253 | NE1 | TRP | 403 | 4.443 | −9.338 | −1.293 | 0.00 | 0.00 | −0.292 | 9.00 | −17.40 |
| ATOM | 1254 | HE1 | TRP | 403 | 5.337 | −8.874 | −1.076 | 0.00 | 0.00 | 0.393 | 0.00 | 0.00 |
| ATOM | 1255 | CE2 | TRP | 403 | 3.629 | −9.984 | −0.386 | 0.00 | 0.00 | −0.050 | 6.80 | 0.60 |
| ATOM | 1256 | CE3 | TRP | 403 | 1.531 | −11.164 | −0.442 | 0.00 | 0.00 | −0.127 | 10.80 | 0.60 |
| ATOM | 1257 | HE3 | TRP | 403 | 0.666 | −11.544 | −0.985 | 0.00 | 0.00 | 0.127 | 0.00 | 0.00 |
| ATOM | 1258 | CZ2 | TRP | 403 | 3.760 | −10.175 | 0.960 | 0.00 | 0.00 | −0.127 | 10.80 | 0.60 |
| ATOM | 1259 | HZ2 | TRP | 403 | 4.622 | −9.790 | 1.505 | 0.00 | 0.00 | 0.127 | 0.00 | 0.00 |
| ATOM | 1260 | CZ3 | TRP | 403 | 1.667 | −11.358 | 0.915 | 0.00 | 0.00 | −0.127 | 10.80 | 0.60 |
| ATOM | 1261 | HZ3 | TRP | 403 | 0.895 | −11.905 | 1.456 | 0.00 | 0.00 | 0.127 | 0.00 | 0.00 |
| ATOM | 1262 | CH2 | TRP | 403 | 2.761 | −10.873 | 1.603 | 0.00 | 0.00 | −0.127 | 10.80 | 0.60 |
| ATOM | 1263 | HH2 | TRP | 403 | 2.836 | −11.046 | 2.677 | 0.00 | 0.00 | 0.127 | 0.00 | 0.00 |
| ATOM | 1264 | N | ASN | 404 | 4.511 | −11.679 | −4.358 | 0.00 | 0.00 | −0.650 | 9.00 | −17.40 |
| ATOM | 1265 | HN | ASN | 404 | 4.411 | −12.173 | −3.460 | 0.00 | 0.00 | 0.439 | 0.00 | 0.00 |
| ATOM | 1266 | CA | ASN | 404 | 5.820 | −11.386 | −4.865 | 0.00 | 0.00 | 0.158 | 9.40 | 4.00 |
| ATOM | 1267 | HA | ASN | 404 | 5.893 | −10.324 | −5.099 | 0.00 | 0.00 | 0.053 | 0.00 | 0.00 |
| ATOM | 1268 | C | ASN | 404 | 6.069 | −12.184 | −6.107 | 0.00 | 0.00 | 0.396 | 9.82 | 4.00 |
| ATOM | 1269 | O | ASN | 404 | 6.571 | −11.671 | −7.108 | 0.00 | 0.00 | −0.396 | 8.17 | −17.40 |
| ATOM | 1270 | CB | ASN | 404 | 6.891 | −11.794 | −3.848 | 0.00 | 0.00 | −0.106 | 12.77 | 4.00 |
| ATOM | 1271 | HB1 | ASN | 404 | 6.933 | −12.882 | −3.789 | 0.00 | 0.00 | 0.053 | 0.00 | 0.00 |
| ATOM | 1272 | HB2 | ASN | 404 | 6.634 | −11.382 | −2.872 | 0.00 | 0.00 | 0.053 | 0.00 | 0.00 |
| ATOM | 1273 | CG | ASN | 404 | 8.231 | −11.254 | −4.294 | 0.00 | 0.00 | 0.396 | 9.82 | 4.00 |
| ATOM | 1274 | OD1 | ASN | 404 | 8.555 | −11.183 | −5.478 | 0.00 | 0.00 | −0.396 | 8.17 | −17.40 |
| ATOM | 1275 | ND2 | ASN | 404 | 9.040 | −10.854 | −3.279 | 0.00 | 0.00 | −0.879 | 13.25 | −17.40 |
| ATOM | 1276 | HD21 | ASN | 404 | 8.720 | −10.935 | −2.303 | 0.00 | 0.00 | 0.439 | 0.00 | 0.00 |
| ATOM | 1277 | HD22 | ASN | 404 | 9.973 | −10.470 | −3.485 | 0.00 | 0.00 | 0.439 | 0.00 | 0.00 |
| ATOM | 1278 | N | LEU | 405 | 5.695 | −13.473 | −6.074 | 0.00 | 0.00 | −0.650 | 9.00 | −17.40 |
| ATOM | 1279 | HN | LEU | 405 | 5.173 | −13.823 | −5.258 | 0.00 | 0.00 | 0.439 | 0.00 | 0.00 |
| ATOM | 1280 | CA | LEU | 405 | 6.002 | −14.375 | −7.144 | 0.00 | 0.00 | 0.158 | 9.40 | 4.00 |
| ATOM | 1281 | HA | LEU | 405 | 7.082 | −14.410 | −7.289 | 0.00 | 0.00 | 0.053 | 0.00 | 0.00 |
| ATOM | 1282 | C | LEU | 405 | 5.345 | −13.900 | −8.396 | 0.00 | 0.00 | 0.396 | 9.82 | 4.00 |
| ATOM | 1283 | O | LEU | 405 | 5.964 | −13.924 | −9.458 | 0.00 | 0.00 | −0.396 | 8.17 | −17.40 |
| ATOM | 1284 | CB | LEU | 405 | 5.523 | −15.816 | −6.846 | 0.00 | 0.00 | −0.106 | 12.77 | 4.00 |
| ATOM | 1285 | HB1 | LEU | 405 | 4.433 | −15.806 | −6.856 | 0.00 | 0.00 | 0.053 | 0.00 | 0.00 |
| ATOM | 1286 | HB2 | LEU | 405 | 5.910 | −16.092 | −5.865 | 0.00 | 0.00 | 0.053 | 0.00 | 0.00 |
| ATOM | 1287 | CG | LEU | 405 | 5.961 | −16.940 | −7.823 | 0.00 | 0.00 | −0.053 | 9.40 | 4.00 |
| ATOM | 1288 | HG | LEU | 405 | 7.049 | −17.007 | −7.850 | 0.00 | 0.00 | 0.053 | 0.00 | 0.00 |
| ATOM | 1289 | CD1 | LEU | 405 | 5.389 | −18.289 | −7.362 | 0.00 | 0.00 | −0.159 | 16.15 | 4.00 |
| ATOM | 1290 | HD11 | LEU | 405 | 5.701 | −19.072 | −8.053 | 0.00 | 0.00 | 0.053 | 0.00 | 0.00 |
| ATOM | 1291 | HD12 | LEU | 405 | 5.759 | −18.519 | −6.363 | 0.00 | 0.00 | 0.053 | 0.00 | 0.00 |
| ATOM | 1292 | HD13 | LEU | 405 | 4.301 | −18.235 | −7.343 | 0.00 | 0.00 | 0.053 | 0.00 | 0.00 |
| ATOM | 1293 | CD2 | LEU | 405 | 5.606 | −16.672 | −9.298 | 0.00 | 0.00 | −0.159 | 16.15 | 4.00 |
| ATOM | 1294 | HD21 | LEU | 405 | 5.947 | −17.506 | −9.912 | 0.00 | 0.00 | 0.053 | 0.00 | 0.00 |
| ATOM | 1295 | HD22 | LEU | 405 | 4.526 | −16.566 | −9.399 | 0.00 | 0.00 | 0.053 | 0.00 | 0.00 |
| ATOM | 1296 | HD23 | LEU | 405 | 6.093 | −15.755 | −9.629 | 0.00 | 0.00 | 0.053 | 0.00 | 0.00 |
| ATOM | 1297 | N | ASN | 406 | 4.078 | −13.452 | −8.322 | 0.00 | 0.00 | −0.650 | 9.00 | −17.40 |
| ATOM | 1298 | HN | ASN | 406 | 3.598 | −13.353 | −7.416 | 0.00 | 0.00 | 0.439 | 0.00 | 0.00 |
| ATOM | 1299 | CA | ASN | 406 | 3.423 | −13.118 | −9.552 | 0.00 | 0.00 | 0.158 | 9.40 | 4.00 |
| ATOM | 1300 | HA | ASN | 406 | 3.425 | −13.984 | −10.214 | 0.00 | 0.00 | 0.053 | 0.00 | 0.00 |
| ATOM | 1301 | C | ASN | 406 | 4.148 | −11.980 | −10.217 | 0.00 | 0.00 | 0.396 | 9.82 | 4.00 |
| ATOM | 1302 | O | ASN | 406 | 4.476 | −12.090 | −11.396 | 0.00 | 0.00 | −0.396 | 8.17 | −17.40 |
| ATOM | 1303 | CB | ASN | 406 | 1.931 | −12.774 | −9.373 | 0.00 | 0.00 | −0.106 | 12.77 | 4.00 |
| ATOM | 1304 | HB1 | ASN | 406 | 1.516 | −12.555 | −10.357 | 0.00 | 0.00 | 0.053 | 0.00 | 0.00 |
| ATOM | 1305 | HB2 | ASN | 406 | 1.863 | −11.905 | −8.719 | 0.00 | 0.00 | 0.053 | 0.00 | 0.00 |
| ATOM | 1306 | CG | ASN | 406 | 1.245 | −13.976 | −8.751 | 0.00 | 0.00 | 0.396 | 9.82 | 4.00 |
| ATOM | 1307 | OD1 | ASN | 406 | 1.285 | −15.088 | −9.275 | 0.00 | 0.00 | −0.396 | 8.17 | −17.40 |
| ATOM | 1308 | ND2 | ASN | 406 | 0.603 | −13.739 | −7.576 | 0.00 | 0.00 | −0.879 | 13.25 | −17.40 |
| ATOM | 1309 | HD21 | ASN | 406 | 0.596 | −12.790 | −7.175 | 0.00 | 0.00 | 0.439 | 0.00 | 0.00 |
| ATOM | 1310 | HD22 | ASN | 406 | 0.123 | −14.508 | −7.087 | 0.00 | 0.00 | 0.439 | 0.00 | 0.00 |
| ATOM | 1311 | N | PRO | 407 | 4.447 | −10.898 | −9.540 | 0.00 | 0.00 | −0.422 | 9.00 | −17.40 |
| ATOM | 1312 | CA | PRO | 407 | 5.217 | −9.900 | −10.205 | 0.00 | 0.00 | 0.158 | 9.40 | 4.00 |
| ATOM | 1313 | HA | PRO | 407 | 4.641 | −9.543 | −11.059 | 0.00 | 0.00 | 0.053 | 0.00 | 0.00 |
| ATOM | 1314 | CD | PRO | 407 | 3.398 | −10.217 | −8.808 | 0.00 | 0.00 | 0.105 | 12.77 | 4.00 |
| ATOM | 1315 | HD1 | PRO | 407 | 3.302 | −10.722 | −7.847 | 0.00 | 0.00 | 0.053 | 0.00 | 0.00 |
| ATOM | 1316 | HD2 | PRO | 407 | 2.489 | −10.301 | −9.404 | 0.00 | 0.00 | 0.053 | 0.00 | 0.00 |
| ATOM | 1317 | C | PRO | 407 | 6.533 | −10.346 | −10.720 | 0.00 | 0.00 | 0.396 | 9.82 | 4.00 |
| ATOM | 1318 | O | PRO | 407 | 6.929 | −9.844 | −11.768 | 0.00 | 0.00 | −0.396 | 8.17 | −17.40 |
| ATOM | 1319 | CB | PRO | 407 | 5.251 | −8.708 | −9.259 | 0.00 | 0.00 | −0.106 | 12.77 | 4.00 |
| ATOM | 1320 | HB1 | PRO | 407 | 5.440 | −7.779 | −9.797 | 0.00 | 0.00 | 0.053 | 0.00 | 0.00 |
| ATOM | 1321 | HB2 | PRO | 407 | 6.035 | −8.818 | −8.509 | 0.00 | 0.00 | 0.053 | 0.00 | 0.00 |
| ATOM | 1322 | CG | PRO | 407 | 3.844 | −8.751 | −8.644 | 0.00 | 0.00 | −0.106 | 12.77 | 4.00 |
| ATOM | 1323 | HG1 | PRO | 407 | 3.266 | −8.043 | −9.238 | 0.00 | 0.00 | 0.053 | 0.00 | 0.00 |
| ATOM | 1324 | HG2 | PRO | 407 | 3.991 | −8.445 | −7.608 | 0.00 | 0.00 | 0.053 | 0.00 | 0.00 |
| ATOM | 1325 | N | GLN | 408 | 7.225 | −11.277 | −10.044 | 0.00 | 0.00 | −0.650 | 9.00 | −17.40 |
| ATOM | 1326 | HN | GLN | 408 | 6.853 | −11.700 | −9.182 | 0.00 | 0.00 | 0.439 | 0.00 | 0.00 |
| ATOM | 1327 | CA | GLN | 408 | 8.500 | −11.658 | −10.570 | 0.00 | 0.00 | 0.158 | 9.40 | 4.00 |
| ATOM | 1328 | HA | GLN | 408 | 9.097 | −10.747 | −10.614 | 0.00 | 0.00 | 0.053 | 0.00 | 0.00 |
| ATOM | 1329 | C | GLN | 408 | 8.240 | −12.234 | −11.918 | 0.00 | 0.00 | 0.396 | 9.82 | 4.00 |
| ATOM | 1330 | O | GLN | 408 | 8.923 | −11.912 | −12.889 | 0.00 | 0.00 | −0.396 | 8.17 | −17.40 |
| ATOM | 1331 | CB | GLN | 408 | 9.211 | −12.737 | −9.723 | 0.00 | 0.00 | −0.106 | 12.77 | 4.00 |
| ATOM | 1332 | HB1 | GLN | 408 | 8.579 | −13.625 | −9.705 | 0.00 | 0.00 | 0.053 | 0.00 | 0.00 |
| ATOM | 1333 | HB2 | GLN | 408 | 9.350 | −12.341 | −8.717 | 0.00 | 0.00 | 0.053 | 0.00 | 0.00 |
| ATOM | 1334 | CG | GLN | 408 | 10.595 | −13.168 | −10.243 | 0.00 | 0.00 | −0.106 | 12.77 | 4.00 |
| ATOM | 1335 | HG1 | GLN | 408 | 11.145 | −13.686 | −9.457 | 0.00 | 0.00 | 0.053 | 0.00 | 0.00 |
| ATOM | 1336 | HG2 | GLN | 408 | 11.165 | −12.293 | −10.555 | 0.00 | 0.00 | 0.053 | 0.00 | 0.00 |
| ATOM | 1337 | CD | GLN | 408 | 10.436 | −14.107 | −11.436 | 0.00 | 0.00 | 0.396 | 9.82 | 4.00 |
| ATOM | 1338 | OE1 | GLN | 408 | 9.567 | −14.978 | −11.442 | 0.00 | 0.00 | −0.396 | 8.17 | −17.40 |
| ATOM | 1339 | NE2 | GLN | 408 | 11.271 | −13.900 | −12.490 | 0.00 | 0.00 | −0.879 | 13.25 | −17.40 |
| ATOM | 1340 | HE21 | GLN | 408 | 11.985 | −13.159 | −12.443 | 0.00 | 0.00 | 0.439 | 0.00 | 0.00 |
| ATOM | 1341 | HE22 | GLN | 408 | 11.189 | −14.484 | −13.334 | 0.00 | 0.00 | 0.439 | 0.00 | 0.00 |
| ATOM | 1342 | N | GLN | 409 | 7.220 | −13.102 | −12.012 | 0.00 | 0.00 | −0.650 | 9.00 | −17.40 |
| ATOM | 1343 | HN | GLN | 409 | 6.639 | −13.319 | −11.190 | 0.00 | 0.00 | 0.439 | 0.00 | 0.00 |
| ATOM | 1344 | CA | GLN | 409 | 6.956 | −13.718 | −13.271 | 0.00 | 0.00 | 0.158 | 9.40 | 4.00 |
| ATOM | 1345 | HA | GLN | 409 | 7.866 | −14.163 | −13.672 | 0.00 | 0.00 | 0.053 | 0.00 | 0.00 |
| ATOM | 1346 | C | GLN | 409 | 6.448 | −12.704 | −14.249 | 0.00 | 0.00 | 0.396 | 9.82 | 4.00 |
| ATOM | 1347 | O | GLN | 409 | 6.953 | −12.617 | −15.364 | 0.00 | 0.00 | −0.396 | 8.17 | −17.40 |
| ATOM | 1348 | CB | GLN | 409 | 5.940 | −14.871 | −13.172 | 0.00 | 0.00 | −0.106 | 12.77 | 4.00 |
| ATOM | 1349 | HB1 | GLN | 409 | 5.655 | −15.267 | −14.147 | 0.00 | 0.00 | 0.053 | 0.00 | 0.00 |
| ATOM | 1350 | HB2 | GLN | 409 | 5.011 | −14.574 | −12.686 | 0.00 | 0.00 | 0.053 | 0.00 | 0.00 |
| ATOM | 1351 | CG | GLN | 409 | 6.473 | −16.064 | −12.371 | 0.00 | 0.00 | −0.106 | 12.77 | 4.00 |
| ATOM | 1352 | HG1 | GLN | 409 | 5.652 | −16.762 | −12.208 | 0.00 | 0.00 | 0.053 | 0.00 | 0.00 |
| ATOM | 1353 | HG2 | GLN | 409 | 6.856 | −15.695 | −11.420 | 0.00 | 0.00 | 0.053 | 0.00 | 0 00 |
| ATOM | 1354 | CD | GLN | 409 | 7.585 | −16.724 | −13.177 | 0.00 | 0.00 | 0.396 | 9.82 | 4.00 |
| ATOM | 1355 | OE1 | GLN | 409 | 7.730 | −16.486 | −14.376 | 0.00 | 0.00 | −0.396 | 8.17 | −17 40 |
| ATOM | 1356 | NE2 | GLN | 409 | 8.392 | −17.588 | −12.505 | 0.00 | 0.00 | −0.879 | 13.25 | −17 40 |
| ATOM | 1357 | HE21 | GLN | 409 | 8.239 | −17.761 | −11.501 | 0.00 | 0.00 | 0.439 | 0.00 | 0.00 |
| ATOM | 1358 | HE22 | GLN | 409 | 9.156 | −18.069 | −13.000 | 0.00 | 0.00 | 0.439 | 0.00 | 0.00 |
| ATOM | 1359 | N | PHE | 410 | 5.466 | −11.862 | −13.881 | 0.00 | 0.00 | −0.650 | 9.00 | −17.40 |
| ATOM | 1360 | HN | PHE | 410 | 5.103 | −11.811 | −12.919 | 0.00 | 0.00 | 0.439 | 0.00 | 0.00 |
| ATOM | 1361 | CA | PHE | 410 | 4.970 | −11.042 | −14.948 | 0.00 | 0.00 | 0.158 | 9.40 | 4.00 |
| ATOM | 1362 | HA | PHE | 410 | 4.680 | −11.622 | −15.824 | 0.00 | 0.00 | 0.053 | 0.00 | 0.00 |
| ATOM | 1363 | C | PHE | 410 | 5.983 | −10.041 | −15.429 | 0.00 | 0.00 | 0.396 | 9.82 | 4.00 |
| ATOM | 1364 | O | PHE | 410 | 6.041 | −9.761 | −16.627 | 0.00 | 0.00 | −0.396 | 8.17 | −17.40 |
| ATOM | 1365 | CB | PHE | 410 | 3.601 | −10.375 | −14.731 | 0.00 | 0.00 | −0.106 | 12.77 | 4.00 |
| ATOM | 1366 | HB1 | PHE | 410 | 2.869 | −11.161 | −14.543 | 0.00 | 0.00 | 0.053 | 0.00 | 0.00 |
| ATOM | 1367 | HB2 | PHE | 410 | 3.350 | −9.818 | −15.634 | 0.00 | 0.00 | 0.053 | 0.00 | 0.00 |
| ATOM | 1368 | CG | PHE | 410 | 3.529 | −9.427 | −13.596 | 0.00 | 0.00 | 0.000 | 7.26 | 0.60 |
| ATOM | 1369 | CD1 | PHE | 410 | 4.191 | −8.224 | −13.641 | 0.00 | 0.00 | −0.127 | 10.80 | 0.60 |
| ATOM | 1370 | HD1 | PHE | 410 | 4.783 | −7.964 | −14.518 | 0.00 | 0.00 | 0.127 | 0.00 | 0.00 |
| ATOM | 1371 | CD2 | PHE | 410 | 2.784 | −9.749 | −12.488 | 0.00 | 0.00 | −0.127 | 10.80 | 0.60 |
| ATOM | 1372 | HD2 | PHE | 410 | 2.259 | −10.703 | −12.445 | 0.00 | 0.00 | 0.127 | 0.00 | 0.00 |
| ATOM | 1373 | CE1 | PHE | 410 | 4.111 | −7.347 | −12.586 | 0.00 | 0.00 | −0.127 | 10.80 | 0.60 |
| ATOM | 1374 | HE1 | PHE | 410 | 4.642 | −6.396 | −12.625 | 0.00 | 0.00 | 0.127 | 0.00 | 0.00 |
| ATOM | 1375 | CE2 | PHE | 410 | 2.698 | −8.874 | −11.436 | 0.00 | 0.00 | −0.127 | 10.80 | 0.60 |
| ATOM | 1376 | HE2 | PHE | 410 | 2.102 | −9.133 | −10.561 | 0.00 | 0.00 | 0.127 | 0.00 | 0.00 |
| ATOM | 1377 | CZ | PHE | 410 | 3.361 | −7.671 | −11.482 | 0.00 | 0.00 | −0.127 | 10.80 | 0.60 |
| ATOM | 1378 | HZ | PHE | 410 | 3.292 | −6.976 | −10.645 | 0.00 | 0.00 | 0.127 | 0.00 | 0.00 |
| ATOM | 1379 | N | TYR | 411 | 6.815 | −9.469 | −14.536 | 0.00 | 0.00 | −0.650 | 9.00 | −17.40 |
| ATOM | 1380 | HN | TYR | 411 | 6.744 | −9.687 | −13.532 | 0.00 | 0.00 | 0.439 | 0.00 | 0.00 |
| ATOM | 1381 | CA | TYR | 411 | 7.806 | −8.545 | −15.023 | 0.00 | 0.00 | 0.158 | 9.40 | 4.00 |
| ATOM | 1382 | HA | TYR | 411 | 7.305 | −7.734 | −15.551 | 0.00 | 0.00 | 0.053 | 0.00 | 0.00 |
| ATOM | 1383 | C | TYR | 411 | 8.732 | −9.270 | −15.956 | 0.00 | 0.00 | 0.396 | 9.82 | 4.00 |
| ATOM | 1384 | O | TYR | 411 | 9.152 | −8.707 | −16.962 | 0.00 | 0.00 | −0.396 | 8.17 | −17.40 |
| ATOM | 1385 | CB | TYR | 411 | 8.775 | −7.956 | −13.970 | 0.00 | 0.00 | −0.106 | 12.77 | 4.00 |
| ATOM | 1386 | HB1 | TYR | 411 | 9.622 | −7.428 | −14.407 | 0.00 | 0.00 | 0.053 | 0.00 | 0.00 |
| ATOM | 1387 | HB2 | TYR | 411 | 9.215 | −8.709 | −13.316 | 0.00 | 0.00 | 0.053 | 0.00 | 0.00 |
| ATOM | 1388 | CG | TYR | 411 | 8.130 | −6.971 | −13.055 | 0.00 | 0.00 | 0.000 | 7.26 | 0.60 |
| ATOM | 1389 | CD1 | TYR | 411 | 7.796 | −5.706 | −13.481 | 0.00 | 0.00 | −0.127 | 10.80 | 0.60 |
| ATOM | 1390 | HD1 | TYR | 411 | 7.993 | −5.414 | −14.513 | 0.00 | 0.00 | 0.127 | 0.00 | 0.00 |
| ATOM | 1391 | CD2 | TYR | 411 | 7.901 | −7.299 | −11.743 | 0.00 | 0.00 | −0.127 | 10.80 | 0.60 |
| ATOM | 1392 | HD2 | TYR | 411 | 8.185 | −8.288 | −11.385 | 0.00 | 0.00 | 0.127 | 0.00 | 0.00 |
| ATOM | 1393 | CE1 | TYR | 411 | 7.216 | −4.806 | −12.617 | 0.00 | 0.00 | −0.127 | 10.80 | 0.60 |
| ATOM | 1394 | HE1 | TYR | 411 | 6.946 | −3.810 | −12.969 | 0.00 | 0.00 | 0.127 | 0.00 | 0.00 |
| ATOM | 1395 | CE2 | TYR | 411 | 7.323 | −6.412 | −10.871 | 0.00 | 0.00 | −0.127 | 10.80 | 0.60 |
| ATOM | 1396 | HE2 | TYR | 411 | 7.141 | −6.700 | −9.836 | 0.00 | 0.00 | 0.127 | 0.00 | 0.00 |
| ATOM | 1397 | CZ | TYR | 411 | 6.976 | −5.160 | −11.311 | 0.00 | 0.00 | 0.027 | 7.26 | 0.60 |
| ATOM | 1398 | OH | TYR | 411 | 6.384 | −4.245 | −10.418 | 0.00 | 0.00 | −0.451 | 10.94 | −17.40 |
| ATOM | 1399 | HH | TYR | 411 | 6.870 | −3.339 | −10.481 | 0.00 | 0.00 | 0.424 | 0.00 | 0.00 |
| ATOM | 1400 | N | THR | 412 | 9.098 | −10.532 | −15.655 | 0.00 | 0.00 | −0.650 | 9.00 | −17.40 |
| ATOM | 1401 | HN | THR | 412 | 8.699 | −11.007 | −14.833 | 0.00 | 0.00 | 0.439 | 0.00 | 0.00 |
| ATOM | 1402 | CA | THR | 412 | 10.051 | −11.209 | −16.492 | 0.00 | 0.00 | 0.158 | 9.40 | 4.00 |
| ATOM | 1403 | HA | THR | 412 | 10.956 | −10.603 | −16.517 | 0.00 | 0.00 | 0.053 | 0.00 | 0.00 |
| ATOM | 1404 | C | THR | 412 | 9.449 | −11.336 | −17.845 | 0.00 | 0.00 | 0.396 | 9.82 | 4.00 |
| ATOM | 1405 | O | THR | 412 | 10.143 | −11.237 | −18.855 | 0.00 | 0.00 | −0.396 | 8.17 | −17.40 |
| ATOM | 1406 | CB | THR | 412 | 10.447 | −12.588 | −16.018 | 0.00 | 0.00 | 0.060 | 9.40 | 4.00 |
| ATOM | 1407 | HB | THR | 412 | 10.645 | −12.581 | −14.946 | 0.00 | 0.00 | 0.053 | 0.00 | 0.00 |
| ATOM | 1408 | OG1 | THR | 412 | 11.624 | −13.011 | −16.691 | 0.00 | 0.00 | −0.537 | 11.04 | −17.40 |
| ATOM | 1409 | HG1 | THR | 412 | 11.620 | −12.645 | −17.654 | 0.00 | 0.00 | 0.424 | 0.00 | 0.00 |
| ATOM | 1410 | CG2 | THR | 412 | 9.322 | −13.597 | −16.298 | 0.00 | 0.00 | −0.159 | 16.15 | 4.00 |
| ATOM | 1411 | HG21 | THR | 412 | 9.624 | −14.585 | −15.950 | 0.00 | 0.00 | 0.053 | 0.00 | 0.00 |
| ATOM | 1412 | HG22 | THR | 412 | 8.418 | −13.289 | −15.773 | 0.00 | 0.00 | 0.053 | 0.00 | 0.00 |
| ATOM | 1413 | HG23 | THR | 412 | 9.125 | −13.634 | −17.369 | 0.00 | 0.00 | 0.053 | 0.00 | 0.00 |
| ATOM | 1414 | N | MET | 413 | 8.126 | −11.560 | −17.894 | 0.00 | 0.00 | −0.650 | 9.00 | −17.40 |
| ATOM | 1415 | HN | MET | 413 | 7.584 | −11.665 | −17.025 | 0.00 | 0.00 | 0.439 | 0.00 | 0.00 |
| ATOM | 1416 | CA | MET | 413 | 7.479 | −11.651 | −19.163 | 0.00 | 0.00 | 0.158 | 9.40 | 4.00 |
| ATOM | 1417 | HA | MET | 413 | 7.932 | −12.467 | −19.726 | 0.00 | 0.00 | 0.053 | 0.00 | 0.00 |
| ATOM | 1418 | C | MET | 413 | 7.663 | −10.358 | −19.884 | 0.00 | 0.00 | 0.396 | 9.82 | 4.00 |
| ATOM | 1419 | O | MET | 413 | 7.962 | −10.368 | −21.070 | 0.00 | 0.00 | −0.396 | 8.17 | −17.40 |
| ATOM | 1420 | CB | MET | 413 | 5.975 | −11.960 | −19.052 | 0.00 | 0.00 | −0.106 | 12.77 | 4.00 |
| ATOM | 1421 | HB1 | MET | 413 | 5.399 | −11.583 | −19.897 | 0.00 | 0.00 | 0.053 | 0.00 | 0.00 |
| ATOM | 1422 | HB2 | MET | 413 | 5.515 | −11.526 | −18.164 | 0.00 | 0.00 | 0.053 | 0.00 | 0.00 |
| ATOM | 1423 | CG | MET | 413 | 5.675 | −13.461 | −18.983 | 0.00 | 0.00 | −0.041 | 12.77 | 4.00 |
| ATOM | 1424 | HG1 | MET | 413 | 6.083 | −13.926 | −19.881 | 0.00 | 0.00 | 0.053 | 0.00 | 0.00 |
| ATOM | 1425 | HG2 | MET | 413 | 4.593 | −13.585 | −18.936 | 0.00 | 0.00 | 0.053 | 0.00 | 0.00 |
| ATOM | 1426 | SD | MET | 413 | 6.374 | −14.344 | −17.561 | 0.00 | 0.00 | −0.130 | 16.39 | −6.40 |
| ATOM | 1427 | CE | MET | 413 | 5.950 | −16.002 | −18.163 | 0.00 | 0.00 | −0.094 | 16.15 | 4.00 |
| ATOM | 1428 | HE1 | MET | 413 | 6.286 | −16.748 | −17.442 | 0.00 | 0.00 | 0.053 | 0.00 | 0.00 |
| ATOM | 1429 | HE2 | MET | 413 | 4.870 | −16.079 | −18.288 | 0.00 | 0.00 | 0.053 | 0.00 | 0.00 |
| ATOM | 1430 | HE3 | MET | 413 | 6.439 | −16.177 | −19.121 | 0.00 | 0.00 | 0.053 | 0.00 | 0.00 |
| ATOM | 1431 | N | PHE | 414 | 7.511 | −9.204 | −19.211 | 0.00 | 0.00 | −0.650 | 9.00 | −17.40 |
| ATOM | 1432 | HN | PHE | 414 | 7.276 | −9.214 | −18.208 | 0.00 | 0.00 | 0.439 | 0.00 | 0.00 |
| ATOM | 1433 | CA | PHE | 414 | 7.680 | −7.957 | −19.903 | 0.00 | 0.00 | 0.158 | 9.40 | 4.00 |
| ATOM | 1434 | HA | PHE | 414 | 6.964 | −7.926 | −20.724 | 0.00 | 0.00 | 0.053 | 0.00 | 0.00 |
| ATOM | 1435 | C | PHE | 414 | 9.036 | −7.733 | −20.487 | 0.00 | 0.00 | 0.396 | 9.82 | 4.00 |
| ATOM | 1436 | O | PHE | 414 | 9.103 | −7.591 | −21.710 | 0.00 | 0.00 | −0.396 | 8.17 | −17.40 |
| ATOM | 1437 | CB | PHE | 414 | 7.256 | −6.753 | −19.068 | 0.00 | 0.00 | −0.106 | 12.77 | 4.00 |
| ATOM | 1438 | HB1 | PHE | 414 | 7.978 | −5.938 | −19.122 | 0.00 | 0.00 | 0.053 | 0.00 | 0.00 |
| ATOM | 1439 | HB2 | PHE | 414 | 7.146 | −7.000 | −18.012 | 0.00 | 0.00 | 0.053 | 0.00 | 0.00 |
| ATOM | 1440 | CG | PHE | 414 | 5.994 | −6.465 | −19.738 | 0.00 | 0.00 | 0.000 | 7.26 | 0.60 |
| ATOM | 1441 | CD1 | PHE | 414 | 5.166 | −7.501 | −20.084 | 0.00 | 0.00 | −0.127 | 10.80 | 0.60 |
| ATOM | 1442 | HD1 | PHE | 414 | 5.448 | −8.526 | −19.844 | 0.00 | 0.00 | 0.127 | 0.00 | 0.00 |
| ATOM | 1443 | CD2 | PHE | 414 | 5.662 | −5.172 | −20.020 | 0.00 | 0.00 | −0.127 | 10.80 | 0.60 |
| ATOM | 1444 | HD2 | PHE | 414 | 6.325 | −4.352 | −19.744 | 0.00 | 0.00 | 0.127 | 0.00 | 0.00 |
| ATOM | 1445 | CE1 | PHE | 414 | 3.987 | −7.252 | −20.729 | 0.00 | 0.00 | −0.127 | 10.80 | 0.60 |
| ATOM | 1446 | HE1 | PHE | 414 | 3.330 | −8.074 | −21.014 | 0.00 | 0.00 | 0.127 | 0.00 | 0.00 |
| ATOM | 1447 | CE2 | PHE | 414 | 4.482 | −4.932 | −20.655 | 0.00 | 0.00 | −0.127 | 10.80 | 0.60 |
| ATOM | 1448 | HE2 | PHE | 414 | 4.199 | −3.905 | −20.884 | 0.00 | 0.00 | 0.127 | 0.00 | 0.00 |
| ATOM | 1449 | CZ | PHE | 414 | 3.640 | −5.956 | −21.013 | 0.00 | 0.00 | −0.127 | 10.80 | 0.60 |
| ATOM | 1450 | HZ | PHE | 414 | 2.700 | −5.740 | −21.521 | 0.00 | 0.00 | 0.127 | 0.00 | 0.00 |
| ATOM | 1451 | N | PRO | 415 | 10.132 | −7.649 | −19.783 | 0.00 | 0.00 | −0.422 | 9.00 | −17.40 |
| ATOM | 1452 | CA | PRO | 415 | 11.291 | −7.499 | −20.602 | 0.00 | 0.00 | 0.158 | 9.40 | 4.00 |
| ATOM | 1453 | HA | PRO | 415 | 11.050 | −6.791 | −21.395 | 0.00 | 0.00 | 0.053 | 0.00 | 0.00 |
| ATOM | 1454 | CD | PRO | 415 | 10.231 | −6.741 | −18.645 | 0.00 | 0.00 | 0.105 | 12.77 | 4.00 |
| ATOM | 1455 | HD1 | PRO | 415 | 9.688 | −7.243 | −17.844 | 0.00 | 0.00 | 0.053 | 0.00 | 0.00 |
| ATOM | 1456 | HD2 | PRO | 415 | 9.760 | −5.819 | −18.987 | 0.00 | 0.00 | 0.053 | 0.00 | 0.00 |
| ATOM | 1457 | C | PRO | 415 | 11.796 | −8.742 | −21.265 | 0.00 | 0.00 | 0.396 | 9.82 | 4.00 |
| ATOM | 1458 | O | PRO | 415 | 12.422 | −8.611 | −22.315 | 0.00 | 0.00 | −0.396 | 8.17 | −17.40 |
| ATOM | 1459 | CB | PRO | 415 | 12.348 | −6.745 | −19.785 | 0.00 | 0.00 | −0.106 | 12.77 | 4.00 |
| ATOM | 1460 | HB1 | PRO | 415 | 12.474 | −5.794 | −20.303 | 0.00 | 0.00 | 0.053 | 0.00 | 0.00 |
| ATOM | 1461 | HB2 | PRO | 415 | 13.231 | −7.383 | −19.818 | 0.00 | 0.00 | 0.053 | 0.00 | 0.00 |
| ATOM | 1462 | CG | PRO | 415 | 11.736 | −6.609 | −18.385 | 0.00 | 0.00 | −0.106 | 12.77 | 4.00 |
| ATOM | 1463 | HG1 | PRO | 415 | 11.987 | −5.647 | −17.939 | 0.00 | 0.00 | 0.053 | 0.00 | 0.00 |
| ATOM | 1464 | HG2 | PRO | 415 | 12.102 | −7.391 | −17.720 | 0.00 | 0.00 | 0.053 | 0.00 | 0.00 |
| ATOM | 1465 | N | HIS | 416 | 11.588 | −9.944 | −20.690 | 0.00 | 0.00 | −0.650 | 9.00 | −17.40 |
| ATOM | 1466 | HN | HIS | 416 | 11.016 | −10.042 | −19.839 | 0.00 | 0.00 | 0.439 | 0.00 | 0.00 |
| ATOM | 1467 | CA | HIS | 416 | 12.201 | −11.082 | −21.319 | 0.00 | 0.00 | 0.158 | 9.40 | 4.00 |
| ATOM | 1468 | HA | HIS | 416 | 13.250 | −10.842 | −21.493 | 0.00 | 0.00 | 0.053 | 0.00 | 0.00 |
| ATOM | 1469 | C | HIS | 416 | 11.625 | −11.502 | −22.637 | 0.00 | 0.00 | 0.396 | 9.82 | 4.00 |
| ATOM | 1470 | O | HIS | 416 | 12.233 | −11.299 | −23.687 | 0.00 | 0.00 | −0.396 | 8.17 | −17.40 |
| ATOM | 1471 | CB | HIS | 416 | 12.174 | −12.360 | −20.461 | 0.00 | 0.00 | −0.106 | 12.77 | 4.00 |
| ATOM | 1472 | HB1 | HIS | 416 | 12.118 | −13.276 | −21.050 | 0.00 | 0.00 | 0.053 | 0.00 | 0.00 |
| ATOM | 1473 | HB2 | HIS | 416 | 11.326 | −12.405 | −19.778 | 0.00 | 0.00 | 0.053 | 0.00 | 0.00 |
| ATOM | 1474 | CG | HIS | 416 | 13.384 | −12.521 | −19.597 | 0.00 | 0.00 | 0.241 | 7.26 | 0.60 |
| ATOM | 1475 | ND1 | HIS | 416 | 13.614 | −13.607 | −18.782 | 0.00 | 0.00 | −0.481 | 9.25 | −17.40 |
| ATOM | 1476 | CD2 | HIS | 416 | 14.471 | −11.715 | −19.454 | 0.00 | 0.00 | −0.177 | 10.80 | 0.60 |
| ATOM | 1477 | HD2 | HIS | 416 | 14.608 | −10.762 | −19.965 | 0.00 | 0.00 | 0.127 | 0.00 | 0.00 |
| ATOM | 1478 | CE1 | HIS | 416 | 14.821 | −13.407 | −18.194 | 0.00 | 0.00 | 0.064 | 10.80 | 0.60 |
| ATOM | 1479 | HE1 | HIS | 416 | 15.278 | −14.104 | −17.491 | 0.00 | 0.00 | 0.127 | 0.00 | 0.00 |
| ATOM | 1480 | NE2 | HIS | 416 | 15.378 | −12.271 | −18.572 | 0.00 | 0.00 | −0.292 | 9.25 | −17.40 |
| ATOM | 1481 | HE2 | HIS | 416 | 16.286 | −11.885 | −18.276 | 0.00 | 0.00 | 0.393 | 0.00 | 0.00 |
| ATOM | 1482 | N | THR | 417 | 10.415 | −12.097 | −22.606 | 0.00 | 0.00 | −0.650 | 9.00 | −17.40 |
| ATOM | 1483 | HN | THR | 417 | 9.839 | −12.059 | −21.753 | 0.00 | 0.00 | 0.439 | 0.00 | 0.00 |
| ATOM | 1484 | CA | THR | 417 | 9.934 | −12.786 | −23.777 | 0.00 | 0.00 | 0.158 | 9.40 | 4.00 |
| ATOM | 1485 | HA | THR | 417 | 10.797 | −13.264 | −24.240 | 0.00 | 0.00 | 0.053 | 0.00 | 0.00 |
| ATOM | 1486 | C | THR | 417 | 9.257 | −12.013 | −24.880 | 0.00 | 0.00 | 0.396 | 9.82 | 4.00 |
| ATOM | 1487 | O | THR | 417 | 9.592 | −12.248 | −26.039 | 0.00 | 0.00 | −0.396 | 8.17 | −17.40 |
| ATOM | 1488 | CB | THR | 417 | 9.008 | −13.926 | −23.465 | 0.00 | 0.00 | 0.060 | 9.40 | 4.00 |
| ATOM | 1489 | HB | THR | 417 | 8.847 | −14.495 | −24.381 | 0.00 | 0.00 | 0.053 | 0.00 | 0.00 |
| ATOM | 1490 | OG1 | THR | 417 | 7.760 | −13.444 | −22.993 | 0.00 | 0.00 | −0.537 | 11.04 | −17.40 |
| ATOM | 1491 | HG1 | THR | 417 | 7.787 | −12.416 | −22.937 | 0.00 | 0.00 | 0.424 | 0.00 | 0.00 |
| ATOM | 1492 | CG2 | THR | 417 | 9.670 | −14.803 | −22.389 | 0.00 | 0.00 | −0.159 | 16.15 | 4.00 |
| ATOM | 1493 | HG21 | THR | 417 | 9.014 | −15.639 | −22.146 | 0.00 | 0.00 | 0.053 | 0.00 | 0.00 |
| ATOM | 1494 | HG22 | THR | 417 | 10.620 | −15.184 | −22.764 | 0.00 | 0.00 | 0.053 | 0.00 | 0.00 |
| ATOM | 1495 | HG23 | THR | 417 | 9.846 | −14.208 | −21.493 | 0.00 | 0.00 | 0.053 | 0.00 | 0.00 |
| ATOM | 1496 | N | PRO | 418 | 8.353 | −11.106 | −24.576 | 0.00 | 0.00 | −0.422 | 9.00 | −17.40 |
| ATOM | 1497 | CA | PRO | 418 | 7.407 | −10.624 | −25.561 | 0.00 | 0.00 | 0.158 | 9.40 | 4.00 |
| ATOM | 1498 | HA | PRO | 418 | 6.707 | −11.430 | −25.783 | 0.00 | 0.00 | 0.053 | 0.00 | 0.00 |
| ATOM | 1499 | CD | PRO | 418 | 8.745 | −10.008 | −23.715 | 0.00 | 0.00 | 0.105 | 12.77 | 4.00 |
| ATOM | 1500 | HD1 | PRO | 418 | 9.778 | −9.773 | −23.973 | 0.00 | 0.00 | 0.053 | 0.00 | 0.00 |
| ATOM | 1501 | HD2 | PRO | 418 | 8.643 | −10.372 | −22.693 | 0.00 | 0.00 | 0.053 | 0.00 | 0.00 |
| ATOM | 1502 | C | PRO | 418 | 8.012 | −10.189 | −26.854 | 0.00 | 0.00 | 0.396 | 9.82 | 4.00 |
| ATOM | 1503 | O | PRO | 418 | 7.589 | −10.650 | −27.910 | 0.00 | 0.00 | −0.396 | 8.17 | −17.40 |
| ATOM | 1504 | CB | PRO | 418 | 6.673 | −9.479 | −24.882 | 0.00 | 0.00 | −0.106 | 12.77 | 4.00 |
| ATOM | 1505 | HB1 | PRO | 418 | 5.842 | −9.843 | −24.278 | 0.00 | 0.00 | 0.053 | 0.00 | 0.00 |
| ATOM | 1506 | HB2 | PRO | 418 | 6.266 | −8.780 | −25.613 | 0.00 | 0.00 | 0.053 | 0.00 | 0.00 |
| ATOM | 1507 | CG | PRO | 418 | 7.779 | −8.849 | −24.029 | 0.00 | 0.00 | −0.106 | 12.77 | 4.00 |
| ATOM | 1508 | HG1 | PRO | 418 | 7.266 | −8.457 | −23.151 | 0.00 | 0.00 | 0.053 | 0.00 | 0.00 |
| ATOM | 1509 | HG2 | PRO | 418 | 8.215 | −8.076 | −24.662 | 0.00 | 0.00 | 0.053 | 0.00 | 0.00 |
| ATOM | 1510 | N | ASP | 419 | 9.001 | −9.299 | −26.774 | 0.00 | 0.00 | −0.650 | 9.00 | −17.40 |
| ATOM | 1511 | HN | ASP | 419 | 9.255 | −8.968 | −25.832 | 0.00 | 0.00 | 0.439 | 0.00 | 0.00 |
| ATOM | 1512 | CA | ASP | 419 | 9.742 | −8.761 | −27.869 | 0.00 | 0.00 | 0.158 | 9.40 | 4.00 |
| ATOM | 1513 | HA | ASP | 419 | 10.333 | −9.520 | −28.381 | 0.00 | 0.00 | 0.053 | 0.00 | 0.00 |
| ATOM | 1514 | C | ASP | 419 | 10.551 | −7.785 | −27.119 | 0.00 | 0.00 | 0.396 | 9.82 | 4.00 |
| ATOM | 1515 | O | ASP | 419 | 10.328 | −6.578 | −27.205 | 0.00 | 0.00 | −0.396 | 8.17 | −17.40 |
| ATOM | 1516 | CB | ASP | 419 | 8.880 | −7.978 | −28.880 | 0.00 | 0.00 | −0.336 | 12.77 | 4.00 |
| ATOM | 1517 | HB1 | ASP | 419 | 8.448 | −7.111 | −28.380 | 0.00 | 0.00 | 0.053 | 0.00 | 0.00 |
| ATOM | 1518 | HB2 | ASP | 419 | 8.087 | −8.629 | −29.249 | 0.00 | 0.00 | 0.053 | 0.00 | 0.00 |
| ATOM | 1519 | CG | ASP | 419 | 9.763 | −7.525 | −30.038 | 0.00 | 0.00 | 0.297 | 9.82 | 4.00 |
| ATOM | 1520 | OD1 | ASP | 419 | 10.919 | −7.110 | −29.763 | 0.00 | 0.00 | −0.534 | 8.17 | −18.95 |
| ATOM | 1521 | OD2 | ASP | 419 | 9.290 | −7.576 | −31.205 | 0.00 | 0.00 | −0.534 | 8.17 | −18.95 |
| ATOM | 1522 | N | ASN | 420 | 11.514 | −8.322 | −26.348 | 0.00 | 0.00 | −0.650 | 9.00 | −17.40 |
| ATOM | 1523 | HN | ASN | 420 | 11.746 | −9.319 | −26.459 | 0.00 | 0.00 | 0.439 | 0.00 | 0.00 |
| ATOM | 1524 | CA | ASN | 420 | 12.221 | −7.550 | −25.382 | 0.00 | 0.00 | 0.158 | 9.40 | 4.00 |
| ATOM | 1525 | HA | ASN | 420 | 12.768 | −8.266 | −24.769 | 0.00 | 0.00 | 0.053 | 0.00 | 0.00 |
| ATOM | 1526 | C | ASN | 420 | 11.185 | −6.809 | −24.615 | 0.00 | 0.00 | 0.396 | 9.82 | 4.00 |
| ATOM | 1527 | O | ASN | 420 | 10.066 | −7.289 | −24.473 | 0.00 | 0.00 | −0.396 | 8.17 | −17.40 |
| ATOM | 1528 | CB | ASN | 420 | 13.354 | −6.633 | −25.912 | 0.00 | 0.00 | −0.106 | 12.77 | 4.00 |
| ATOM | 1529 | HB1 | ASN | 420 | 14.117 | −7.185 | −26.461 | 0.00 | 0.00 | 0.053 | 0.00 | 0.00 |
| ATOM | 1530 | HB2 | ASN | 420 | 13.876 | −6.107 | −25.112 | 0.00 | 0.00 | 0.053 | 0.00 | 0.00 |
| ATOM | 1531 | CG | ASN | 420 | 12.842 | −5.561 | −26.860 | 0.00 | 0.00 | 0.396 | 9.82 | 4.00 |
| ATOM | 1532 | OD1 | ASN | 420 | 12.028 | −4.719 | −26.486 | 0.00 | 0.00 | −0.396 | 8.17 | −17.40 |
| ATOM | 1533 | ND2 | ASN | 420 | 13.345 | −5.582 | −28.124 | 0.00 | 0.00 | −0.879 | 13.25 | −17.40 |
| ATOM | 1534 | HD21 | ASN | 420 | 14.026 | −6.306 | −28.395 | 0.00 | 0.00 | 0.439 | 0.00 | 0.00 |
| ATOM | 1535 | HD22 | ASN | 420 | 13.045 | −4.874 | −28.809 | 0.00 | 0.00 | 0.439 | 0.00 | 0.00 |
| ATOM | 1536 | N | SER | 421 | 11.496 | −5.628 | −24.086 | 0.00 | 0.00 | −0.650 | 9.00 | −17.40 |
| ATOM | 1537 | HN | SER | 421 | 12.397 | −5.177 | −24.300 | 0.00 | 0.00 | 0.439 | 0.00 | 0.00 |
| ATOM | 1538 | CA | SER | 421 | 10.557 | −5.001 | −23.217 | 0.00 | 0.00 | 0.158 | 9.40 | 4.00 |
| ATOM | 1539 | HA | SER | 421 | 9.695 | −5.653 | −23.074 | 0.00 | 0.00 | 0.053 | 0.00 | 0.00 |
| ATOM | 1540 | C | SER | 421 | 10.107 | −3.724 | −23.807 | 0.00 | 0.00 | 0.396 | 9.82 | 4.00 |
| ATOM | 1541 | O | SER | 421 | 9.481 | −3.694 | −24.858 | 0.00 | 0.00 | −0.396 | 8.17 | −17.40 |
| ATOM | 1542 | CB | SER | 421 | 11.227 | −4.717 | −21.871 | 0.00 | 0.00 | 0.007 | 12.77 | 4.00 |
| ATOM | 1543 | HB1 | SER | 421 | 11.303 | −5.656 | −21.323 | 0.00 | 0.00 | 0.053 | 0.00 | 0.00 |
| ATOM | 1544 | HB2 | SER | 421 | 10.609 | −4.003 | −21.327 | 0.00 | 0.00 | 0.053 | 0.00 | 0.00 |
| ATOM | 1545 | OG | SER | 421 | 12.516 | −4.177 | −22.122 | 0.00 | 0.00 | −0.537 | 11.04 | −17.40 |
| ATOM | 1546 | HG | SER | 421 | 13.197 | −4.599 | −21.475 | 0.00 | 0.00 | 0.424 | 0.00 | 0.00 |
| ATOM | 1547 | N | PHE | 422 | 10.398 | −2.647 | −23.070 | 0.00 | 0.00 | −0.650 | 9.00 | −17.40 |
| ATOM | 1548 | HN | PHE | 422 | 10.940 | −2.826 | −22.212 | 0.00 | 0.00 | 0.439 | 0.00 | 0.00 |
| ATOM | 1549 | CA | PHE | 422 | 10.058 | −1.285 | −23.314 | 0.00 | 0.00 | 0.158 | 9.40 | 4.00 |
| ATOM | 1550 | HA | PHE | 422 | 10.678 | −0.855 | −24.101 | 0.00 | 0.00 | 0.053 | 0.00 | 0.00 |
| ATOM | 1551 | C | PHE | 422 | 10.313 | −0.672 | −22.000 | 0.00 | 0.00 | 0.396 | 9.82 | 4.00 |
| ATOM | 1552 | O | PHE | 422 | 11.432 | −0.504 | −21.522 | 0.00 | 0.00 | −0.396 | 8.17 | −17.40 |
| ATOM | 1553 | CB | PHE | 422 | 8.536 | −0.990 | −23.401 | 0.00 | 0.00 | −0.106 | 12.77 | 4.00 |
| ATOM | 1554 | HB1 | PHE | 422 | 8.381 | 0.022 | −23.026 | 0.00 | 0.00 | 0.053 | 0.00 | 0.00 |
| ATOM | 1555 | HB2 | PHE | 422 | 8.028 | −1.730 | −22.782 | 0.00 | 0.00 | 0.053 | 0.00 | 0.00 |
| ATOM | 1556 | CG | PHE | 422 | 7.872 | −1.042 | −24.739 | 0.00 | 0.00 | 0.000 | 7.26 | 0.60 |
| ATOM | 1557 | CD1 | PHE | 422 | 7.840 | 0.096 | −25.511 | 0.00 | 0.00 | −0.127 | 10.80 | 0.60 |
| ATOM | 1558 | HD1 | PHE | 422 | 8.314 | 1.005 | −25.140 | 0.00 | 0.00 | 0.127 | 0.00 | 0.00 |
| ATOM | 1559 | CD2 | PHE | 422 | 7.235 | −2.168 | −25.207 | 0.00 | 0.00 | −0.127 | 10.80 | 0.60 |
| ATOM | 1560 | HD2 | PHE | 422 | 7.220 | −3.072 | −24.599 | 0.00 | 0.00 | 0.127 | 0.00 | 0.00 |
| ATOM | 1561 | CE1 | PHE | 422 | 7.224 | 0.113 | −26.738 | 0.00 | 0.00 | −0.127 | 10.80 | 0.60 |
| ATOM | 1562 | HE1 | PHE | 422 | 7.220 | 1.023 | −27.337 | 0.00 | 0.00 | 0.127 | 0.00 | 0.00 |
| ATOM | 1563 | CE2 | PHE | 422 | 6.617 | −2.163 | −26.436 | 0.00 | 0.00 | −0.127 | 10.80 | 0.60 |
| ATOM | 1564 | HE2 | PHE | 422 | 6.129 | −3.066 | −26.802 | 0.00 | 0.00 | 0.127 | 0.00 | 0.00 |
| ATOM | 1565 | CZ | PHE | 422 | 6.613 | −1.023 | −27.205 | 0.00 | 0.00 | −0.127 | 10.80 | 0.60 |
| ATOM | 1566 | HZ | PHE | 422 | 6.127 | −1.022 | −28.181 | 0.00 | 0.00 | 0.127 | 0.00 | 0.00 |
| ATOM | 1567 | N | LEU | 423 | 9.167 | −0.353 | −21.405 | 0.00 | 0.00 | −0.650 | 9.00 | −17.40 |
| ATOM | 1568 | HN | LEU | 423 | 8.317 | −0.556 | −21.950 | 0.00 | 0.00 | 0.439 | 0.00 | 0.00 |
| ATOM | 1569 | CA | LEU | 423 | 8.946 | 0.224 | −20.133 | 0.00 | 0.00 | 0.158 | 9.40 | 4.00 |
| ATOM | 1570 | HA | LEU | 423 | 9.722 | −0.038 | −19.413 | 0.00 | 0.00 | 0.053 | 0.00 | 0.00 |
| ATOM | 1571 | C | LEU | 423 | 7.632 | −0.412 | −19.872 | 0.00 | 0.00 | 0.396 | 9.82 | 4.00 |
| ATOM | 1572 | O | LEU | 423 | 7.130 | −1.114 | −20.748 | 0.00 | 0.00 | −0.396 | 8.17 | −17.40 |
| ATOM | 1573 | CB | LEU | 423 | 8.670 | 1.738 | −20.217 | 0.00 | 0.00 | −0.106 | 12.77 | 4.00 |
| ATOM | 1574 | HB1 | LEU | 423 | 8.696 | 2.144 | −19.206 | 0.00 | 0.00 | 0.053 | 0.00 | 0.00 |
| ATOM | 1575 | HB2 | LEU | 423 | 7.687 | 1.879 | −20.665 | 0.00 | 0.00 | 0.053 | 0.00 | 0.00 |
| ATOM | 1576 | CG | LEU | 423 | 9.685 | 2.543 | −21.065 | 0.00 | 0.00 | −0.053 | 9.40 | 4.00 |
| ATOM | 1577 | HG | LEU | 423 | 10.714 | 2.329 | −20.775 | 0.00 | 0.00 | 0.053 | 0.00 | 0.00 |
| ATOM | 1578 | CD1 | LEU | 423 | 9.561 | 2.218 | −22.565 | 0.00 | 0.00 | −0.159 | 16.15 | 4.00 |
| ATOM | 1579 | HD11 | LEU | 423 | 10.290 | 2.803 | −23.126 | 0.00 | 0.00 | 0.053 | 0.00 | 0.00 |
| ATOM | 1580 | HD12 | LEU | 423 | 9.749 | 1.156 | −22.724 | 0.00 | 0.00 | 0.053 | 0.00 | 0.00 |
| ATOM | 1581 | HD13 | LEU | 423 | 8.556 | 2.464 | −22.909 | 0.00 | 0.00 | 0.053 | 0.00 | 0.00 |
| ATOM | 1582 | CD2 | LEU | 423 | 9.561 | 4.052 | −20.808 | 0.00 | 0.00 | −0.159 | 16.15 | 4.00 |
| ATOM | 1583 | HD21 | LEU | 423 | 10.288 | 4.586 | −21.420 | 0.00 | 0.00 | 0.053 | 0.00 | 0.00 |
| ATOM | 1584 | HD22 | LEU | 423 | 8.556 | 4.384 | −21.067 | 0.00 | 0.00 | 0.053 | 0.00 | 0.00 |
| ATOM | 1585 | HD23 | LEU | 423 | 9.751 | 4.259 | −19.755 | 0.00 | 0.00 | 0.053 | 0.00 | 0.00 |
| ATOM | 1586 | N | GLY | 424 | 7.032 | −0.231 | −18.693 | 0.00 | 0.00 | −0.650 | 9.00 | −17.40 |
| ATOM | 1587 | HN | GLY | 424 | 7.464 | 0.310 | −17.930 | 0.00 | 0.00 | 0.439 | 0.00 | 0.00 |
| ATOM | 1588 | CA | GLY | 424 | 5.747 | −0.843 | −18.568 | 0.00 | 0.00 | 0.105 | 9.40 | 4.00 |
| ATOM | 1589 | HA1 | GLY | 424 | 5.910 | −1.905 | −18.385 | 0.00 | 0.00 | 0.053 | 0.00 | 0.00 |
| ATOM | 1590 | HA2 | GLY | 424 | 5.215 | −0.675 | −19.505 | 0.00 | 0.00 | 0.053 | 0.00 | 0.00 |
| ATOM | 1591 | C | GLY | 424 | 5.058 | −0.193 | −17.426 | 0.00 | 0.00 | 0.396 | 9.82 | 4.00 |
| ATOM | 1592 | O | GLY | 424 | 5.705 | 0.205 | −16.456 | 0.00 | 0.00 | −0.396 | 8.17 | −17.40 |
| ATOM | 1593 | N | PHE | 425 | 3.722 | −0.031 | −17.508 | 0.00 | 0.00 | −0.650 | 9.00 | −17.40 |
| ATOM | 1594 | HN | PHE | 425 | 3.167 | −0.263 | −18.344 | 0.00 | 0.00 | 0.439 | 0.00 | 0.00 |
| ATOM | 1595 | CA | PHE | 425 | 3.163 | 0.498 | −16.305 | 0.00 | 0.00 | 0.158 | 9.40 | 4.00 |
| ATOM | 1596 | HA | PHE | 425 | 4.013 | 0.766 | −15.677 | 0.00 | 0.00 | 0.053 | 0.00 | 0.00 |
| ATOM | 1597 | C | PHE | 425 | 2.337 | −0.599 | −15.727 | 0.00 | 0.00 | 0.396 | 9.82 | 4.00 |
| ATOM | 1598 | O | PHE | 425 | 1.537 | −1.237 | −16.413 | 0.00 | 0.00 | −0.396 | 8.17 | −17.40 |
| ATOM | 1599 | CB | PHE | 425 | 2.388 | 1.843 | −16.398 | 0.00 | 0.00 | −0.106 | 12.77 | 4.00 |
| ATOM | 1600 | HB1 | PHE | 425 | 2.850 | 2.433 | −17.190 | 0.00 | 0.00 | 0.053 | 0.00 | 0.00 |
| ATOM | 1601 | HB2 | PHE | 425 | 2.475 | 2.338 | −15.431 | 0.00 | 0.00 | 0.053 | 0.00 | 0.00 |
| ATOM | 1602 | CG | PHE | 425 | 0.928 | 1.783 | −16.713 | 0.00 | 0.00 | 0.000 | 7.26 | 0.60 |
| ATOM | 1603 | CD1 | PHE | 425 | 0.048 | 1.561 | −15.678 | 0.00 | 0.00 | −0.127 | 10.80 | 0.60 |
| ATOM | 1604 | HD1 | PHE | 425 | 0.435 | 1.415 | −14.670 | 0.00 | 0.00 | 0.127 | 0.00 | 0.00 |
| ATOM | 1605 | CD2 | PHE | 425 | 0.423 | 2.001 | −17.977 | 0.00 | 0.00 | −0.127 | 10.80 | 0.60 |
| ATOM | 1606 | HD2 | PHE | 425 | 1.102 | 2.207 | −18.805 | 0.00 | 0.00 | 0.127 | 0.00 | 0.00 |
| ATOM | 1607 | CE1 | PHE | 425 | −1.308 | 1.520 | −15.891 | 0.00 | 0.00 | −0.127 | 10.80 | 0.60 |
| ATOM | 1608 | HE1 | PHE | 425 | −1.988 | 1.332 | −15.060 | 0.00 | 0.00 | 0.127 | 0.00 | 0.00 |
| ATOM | 1609 | CE2 | PHE | 425 | −0.937 | 1.960 | −18.199 | 0.00 | 0.00 | −0.127 | 10.80 | 0.60 |
| ATOM | 1610 | HE2 | PHE | 425 | −1.328 | 2.121 | −19.204 | 0.00 | 0.00 | 0.127 | 0.00 | 0.00 |
| ATOM | 1611 | CZ | PHE | 425 | −1.802 | 1.717 | −17.157 | 0.00 | 0.00 | −0.127 | 10.80 | 0.60 |
| ATOM | 1612 | HZ | PHE | 425 | −2.877 | 1.681 | −17.336 | 0.00 | 0.00 | 0.127 | 0.00 | 0.00 |
| ATOM | 1613 | N | VAL | 426 | 2.552 | −0.875 | −14.428 | 0.00 | 0.00 | −0.650 | 9.00 | −17.40 |
| ATOM | 1614 | HN | VAL | 426 | 3.190 | −0.297 | −13.862 | 0.00 | 0.00 | 0.439 | 0.00 | 0.00 |
| ATOM | 1615 | CA | VAL | 426 | 1.871 | −1.992 | −13.856 | 0.00 | 0.00 | 0.158 | 9.40 | 4.00 |
| ATOM | 1616 | HA | VAL | 426 | 1.595 | −2.690 | −14.647 | 0.00 | 0.00 | 0.053 | 0.00 | 0.00 |
| ATOM | 1617 | C | VAL | 426 | 0.645 | −1.517 | −13.158 | 0.00 | 0.00 | 0.396 | 9.82 | 4.00 |
| ATOM | 1618 | O | VAL | 426 | 0.687 | −0.584 | −12.354 | 0.00 | 0.00 | −0.396 | 8.17 | −17.40 |
| ATOM | 1619 | CB | VAL | 426 | 2.697 | −2.798 | −12.894 | 0.00 | 0.00 | −0.053 | 9.40 | 4.00 |
| ATOM | 1620 | HB | VAL | 426 | 3.662 | −3.030 | −13.345 | 0.00 | 0.00 | 0.053 | 0.00 | 0.00 |
| ATOM | 1621 | CG1 | VAL | 426 | 2.920 | −1.992 | −11.603 | 0.00 | 0.00 | −0.159 | 16.15 | 4.00 |
| ATOM | 1622 | HG11 | VAL | 426 | 3.519 | −2.579 | −10.907 | 0.00 | 0.00 | 0.053 | 0.00 | 0.00 |
| ATOM | 1623 | HG12 | VAL | 426 | 3.442 | −1.064 | −11.839 | 0.00 | 0.00 | 0.053 | 0.00 | 0.00 |
| ATOM | 1624 | HG13 | VAL | 426 | 1.957 | −1.761 | −11.147 | 0.00 | 0.00 | 0.053 | 0.00 | 0.00 |
| ATOM | 1625 | CG2 | VAL | 426 | 1.986 | −4.141 | −12.685 | 0.00 | 0.00 | −0.159 | 16.15 | 4.00 |
| ATOM | 1626 | HG21 | VAL | 426 | 2.561 | −4.753 | −11.990 | 0.00 | 0.00 | 0.053 | 0.00 | 0.00 |
| ATOM | 1627 | HG22 | VAL | 426 | 0.991 | −3.966 | −12.277 | 0.00 | 0.00 | 0.053 | 0.00 | 0.00 |
| ATOM | 1628 | HG23 | VAL | 426 | 1.901 | −4.660 | −13.640 | 0.00 | 0.00 | 0.053 | 0.00 | 0.00 |
| ATOM | 1629 | N | VAL | 427 | −0.503 | −2.148 | −13.476 | 0.00 | 0.00 | −0.650 | 9.00 | −17.40 |
| ATOM | 1630 | HN | VAL | 427 | −0.519 | −2.918 | −14.160 | 0.00 | 0.00 | 0.439 | 0.00 | 0.00 |
| ATOM | 1631 | CA | VAL | 427 | −1.696 | −1.708 | −12.827 | 0.00 | 0.00 | 0.158 | 9.40 | 4.00 |
| ATOM | 1632 | HA | VAL | 427 | −1.407 | −1.053 | −12.005 | 0.00 | 0.00 | 0.053 | 0.00 | 0.00 |
| ATOM | 1633 | C | VAL | 427 | −2.417 | −2.919 | −12.322 | 0.00 | 0.00 | 0.396 | 9.82 | 4.00 |
| ATOM | 1634 | O | VAL | 427 | −2.628 | −3.898 | −13.039 | 0.00 | 0.00 | −0.396 | 8.17 | −17.40 |
| ATOM | 1635 | CB | VAL | 427 | −2.584 | −0.904 | −13.743 | 0.00 | 0.00 | −0.053 | 9.40 | 4.00 |
| ATOM | 1636 | HB | VAL | 427 | −2.050 | −0.589 | −14.639 | 0.00 | 0.00 | 0.053 | 0.00 | 0.00 |
| ATOM | 1637 | CG1 | VAL | 427 | −3.806 | −1.718 | −14.197 | 0.00 | 0.00 | −0.159 | 16.15 | 4.00 |
| ATOM | 1638 | HG11 | VAL | 427 | −4.425 | −1.109 | −14.856 | 0.00 | 0.00 | 0.053 | 0.00 | 0.00 |
| ATOM | 1639 | HG12 | VAL | 427 | −3.473 | −2.607 | −14.732 | 0.00 | 0.00 | 0.053 | 0.00 | 0.00 |
| ATOM | 1640 | HG13 | VAL | 427 | −4.389 | −2.016 | −13.325 | 0.00 | 0.00 | 0.053 | 0.00 | 0.00 |
| ATOM | 1641 | CG2 | VAL | 427 | −2.854 | 0.451 | −13.079 | 0.00 | 0.00 | −0.159 | 16.15 | 4.00 |
| ATOM | 1642 | HG21 | VAL | 427 | −3.495 | 1.051 | −13.724 | 0.00 | 0.00 | 0.053 | 0.00 | 0.00 |
| ATOM | 1643 | HG22 | VAL | 427 | −3.349 | 0.294 | −12.120 | 0.00 | 0.00 | 0.053 | 0.00 | 0.00 |
| ATOM | 1644 | HG23 | VAL | 427 | −1.910 | 0.972 | −12.919 | 0.00 | 0.00 | 0.053 | 0.00 | 0.00 |
| ATOM | 1645 | N | GLU | 428 | −2.809 | −2.885 | −11.036 | 0.00 | 0.00 | −0.650 | 9.00 | −17.40 |
| ATOM | 1646 | HN | GLU | 428 | −2.637 | −2.048 | −10.461 | 0.00 | 0.00 | 0.439 | 0.00 | 0.00 |
| ATOM | 1647 | CA | GLU | 428 | −3.466 | −4.023 | −10.477 | 0.00 | 0.00 | 0.158 | 9.40 | 4.00 |
| ATOM | 1648 | HA | GLU | 428 | −2.985 | −4.927 | −10.850 | 0.00 | 0.00 | 0.053 | 0.00 | 0.00 |
| ATOM | 1649 | C | GLU | 428 | −4.909 | −4.002 | −10.887 | 0.00 | 0.00 | 0.396 | 9.82 | 4.00 |
| ATOM | 1650 | O | GLU | 428 | −5.448 | −2.995 | −11.343 | 0.00 | 0.00 | −0.396 | 8.17 | −17.40 |
| ATOM | 1651 | CB | GLU | 428 | −3.332 | −4.129 | −8.945 | 0.00 | 0.00 | −0.106 | 12.77 | 4.00 |
| ATOM | 1652 | HB1 | GLU | 428 | −2.714 | −4.978 | −8.654 | 0.00 | 0.00 | 0.053 | 0.00 | 0.00 |
| ATOM | 1653 | HB2 | GLU | 428 | −4.302 | −4.256 | −8.465 | 0.00 | 0.00 | 0.053 | 0.00 | 0.00 |
| ATOM | 1654 | CG | GLU | 428 | −2.689 | −2.871 | −8.346 | 0.00 | 0.00 | −0.336 | 12.77 | 4.00 |
| ATOM | 1655 | HG1 | GLU | 428 | −3.469 | −2.164 | −8.063 | 0.00 | 0.00 | 0.053 | 0.00 | 0.00 |
| ATOM | 1656 | HG2 | GLU | 428 | −2.033 | −2.412 | −9.085 | 0.00 | 0.00 | 0.053 | 0.00 | 0.00 |
| ATOM | 1657 | CD | GLU | 428 | −1.877 | −3.254 | −7.112 | 0.00 | 0.00 | 0.297 | 9.82 | 4.00 |
| ATOM | 1658 | OE1 | GLU | 428 | −1.264 | −4.353 | −7.111 | 0.00 | 0.00 | −0.534 | 8.17 | −18.95 |
| ATOM | 1659 | OE2 | GLU | 428 | −1.869 | −2.441 | −6.147 | 0.00 | 0.00 | −0.534 | 8.17 | −18.95 |
| ATOM | 1660 | N | GLN | 429 | −5.566 | −5.166 | −10.716 | 0.00 | 0.00 | −0.650 | 9.00 | −17.40 |
| ATOM | 1661 | HN | GLN | 429 | −5.044 | −5.937 | −10.275 | 0.00 | 0.00 | 0.439 | 0.00 | 0.00 |
| ATOM | 1662 | CA | GLN | 429 | −6.928 | −5.431 | −11.093 | 0.00 | 0.00 | 0.158 | 9.40 | 4.00 |
| ATOM | 1663 | HA | GLN | 429 | −7.046 | −5.312 | −12.170 | 0.00 | 0.00 | 0.053 | 0.00 | 0.00 |
| ATOM | 1664 | C | GLN | 429 | −7.845 | −4.479 | −10.395 | 0.00 | 0.00 | 0.396 | 9.82 | 4.00 |
| ATOM | 1665 | O | GLN | 429 | −8.883 | −4.085 | −10.928 | 0.00 | 0.00 | −0.396 | 8.17 | −17.40 |
| ATOM | 1666 | CB | GLN | 429 | −7.356 | −6.825 | −10.618 | 0.00 | 0.00 | −0.106 | 12.77 | 4.00 |
| ATOM | 1667 | HB1 | GLN | 429 | −7.081 | −6.930 | −9.568 | 0.00 | 0.00 | 0.053 | 0.00 | 0.00 |
| ATOM | 1668 | HB2 | GLN | 429 | −6.839 | −7.569 | −11.225 | 0.00 | 0.00 | 0.053 | 0.00 | 0.00 |
| ATOM | 1669 | CG | GLN | 429 | −8.855 | −7.087 | −10.736 | 0.00 | 0.00 | −0.106 | 12.77 | 4.00 |
| ATOM | 1670 | HG1 | GLN | 429 | −9.054 | −7.299 | −11.786 | 0.00 | 0.00 | 0.053 | 0.00 | 0.00 |
| ATOM | 1671 | HG2 | GLN | 429 | −9.359 | −6.181 | −10.398 | 0.00 | 0.00 | 0.053 | 0.00 | 0.00 |
| ATOM | 1672 | CD | GLN | 429 | −9.162 | −8.280 | −9.843 | 0.00 | 0.00 | 0.396 | 9.82 | 4.00 |
| ATOM | 1673 | OE1 | GLN | 429 | −8.401 | −9.244 | −9.778 | 0.00 | 0.00 | −0.396 | 8.17 | −17.40 |
| ATOM | 1674 | NE2 | GLN | 429 | −10.313 | −8.209 | −9.124 | 0.00 | 0.00 | −0.879 | 13.25 | −17.40 |
| ATOM | 1675 | HE21 | GLN | 429 | −10.922 | −7.383 | −9.208 | 0.00 | 0.00 | 0.439 | 0.00 | 0.00 |
| ATOM | 1676 | HE22 | GLN | 429 | −10.576 | −8.981 | −8.495 | 0.00 | 0.00 | 0.439 | 0.00 | 0.00 |
| ATOM | 1677 | N | HIS | 430 | −7.476 | −4.131 | −9.156 | 0.00 | 0.00 | −0.650 | 9.00 | −17.40 |
| ATOM | 1678 | HN | HIS | 430 | −6.554 | −4.465 | −8.842 | 0.00 | 0.00 | 0.439 | 0.00 | 0.00 |
| ATOM | 1679 | CA | HIS | 430 | −8.221 | −3.346 | −8.219 | 0.00 | 0.00 | 0.158 | 9.40 | 4.00 |
| ATOM | 1680 | HA | HIS | 430 | −9.234 | −3.725 | −8.081 | 0.00 | 0.00 | 0.053 | 0.00 | 0.00 |
| ATOM | 1681 | C | HIS | 430 | −8.350 | −1.912 | −8.660 | 0.00 | 0.00 | 0.396 | 9.82 | 4.00 |
| ATOM | 1682 | O | HIS | 430 | −9.312 | −1.238 | −8.302 | 0.00 | 0.00 | −0.396 | 8.17 | −17.40 |
| ATOM | 1683 | CB | HIS | 430 | −7.503 | −3.356 | −6.867 | 0.00 | 0.00 | −0.106 | 12.77 | 4.00 |
| ATOM | 1684 | HB1 | HIS | 430 | −8.216 | −3.198 | −6.058 | 0.00 | 0.00 | 0.053 | 0.00 | 0.00 |
| ATOM | 1685 | HB2 | HIS | 430 | −6.754 | −2.565 | −6.830 | 0.00 | 0.00 | 0.053 | 0.00 | 0.00 |
| ATOM | 1686 | CG | HIS | 430 | −6.816 | −4.677 | −6.649 | 0.00 | 0.00 | 0.241 | 7.26 | 0.60 |
| ATOM | 1687 | ND1 | HIS | 430 | −7.466 | −5.879 | −6.472 | 0.00 | 0.00 | −0.481 | 9.25 | −17.40 |
| ATOM | 1688 | CD2 | HIS | 430 | −5.486 | −4.968 | −6.613 | 0.00 | 0.00 | −0.177 | 10.80 | 0.60 |
| ATOM | 1689 | HD2 | HIS | 430 | −4.688 | −4.234 | −6.723 | 0.00 | 0.00 | 0.127 | 0.00 | 0.00 |
| ATOM | 1690 | CE1 | HIS | 430 | −6.505 | −6.828 | −6.338 | 0.00 | 0.00 | 0.064 | 10.80 | 0.60 |
| ATOM | 1691 | HE1 | HIS | 430 | −6.714 | −7.886 | −6.183 | 0.00 | 0.00 | 0.127 | 0.00 | 0.00 |
| ATOM | 1692 | NE2 | HIS | 430 | −5.287 | −6.323 | −6.417 | 0.00 | 0.00 | −0.292 | 9.25 | −17.40 |
| ATOM | 1693 | HE2 | HIS | 430 | −4.390 | −6.825 | −6.348 | 0.00 | 0.00 | 0.393 | 0.00 | 0.00 |
| ATOM | 1694 | N | LEU | 431 | −7.384 | −1.383 | −9.429 | 0.00 | 0.00 | −0.650 | 9.00 | −17.40 |
| ATOM | 1695 | HN | LEU | 431 | −6.649 | −1.981 | −9.833 | 0.00 | 0.00 | 0.439 | 0.00 | 0.00 |
| ATOM | 1696 | CA | LEU | 431 | −7.394 | 0.035 | −9.678 | 0.00 | 0.00 | 0.158 | 9.40 | 4.00 |
| ATOM | 1697 | HA | LEU | 431 | −7.813 | 0.606 | −8.849 | 0.00 | 0.00 | 0.053 | 0.00 | 0.00 |
| ATOM | 1698 | C | LEU | 431 | −3.201 | 0.406 | −10.885 | 0.00 | 0.00 | 0.396 | 9.82 | 4.00 |
| ATOM | 1699 | O | LEU | 431 | −8.345 | −0.364 | −11.335 | 0.00 | 0.00 | −0.396 | 8.17 | −17.40 |
| ATOM | 1700 | CB | LEU | 431 | −5.978 | 0.591 | −9.892 | 0.00 | 0.00 | −0.106 | 12.77 | 4.00 |
| ATOM | 1701 | HB1 | LEU | 431 | −5.986 | 1.677 | −9.990 | 0.00 | 0.00 | 0.053 | 0.00 | 0.00 |
| ATOM | 1702 | HB2 | LEU | 431 | −5.522 | 0.186 | −10.795 | 0.00 | 0.00 | 0.053 | 0.00 | 0.00 |
| ATOM | 1703 | CG | LEU | 431 | −5.051 | 0.243 | −8.713 | 0.00 | 0.00 | −0.053 | 9.40 | 4.00 |
| ATOM | 1704 | HG | LEU | 431 | −4.842 | −0.825 | −8.652 | 0.00 | 0.00 | 0.053 | 0.00 | 0.00 |
| ATOM | 1705 | CD1 | LEU | 431 | −3.693 | 0.947 | −8.813 | 0.00 | 0.00 | −0.159 | 16.15 | 4.00 |
| ATOM | 1706 | HD11 | LEU | 431 | −3.075 | 0.669 | −7.959 | 0.00 | 0.00 | 0.053 | 0.00 | 0.00 |
| ATOM | 1707 | HD12 | LEU | 431 | −3.194 | 0.646 | −9.734 | 0.00 | 0.00 | 0.053 | 0.00 | 0.00 |
| ATOM | 1708 | HD13 | LEU | 431 | −3.842 | 2.027 | −8.817 | 0.00 | 0.00 | 0.053 | 0.00 | 0.00 |
| ATOM | 1709 | CD2 | LEU | 431 | −5.745 | 0.497 | −7.370 | 0.00 | 0.00 | −0.159 | 16.15 | 4.00 |
| ATOM | 1710 | HD21 | LEU | 431 | −5.066 | 0.242 | −6.556 | 0.00 | 0.00 | 0.053 | 0.00 | 0.00 |
| ATOM | 1711 | HD22 | LEU | 431 | −6.021 | 1.549 | −7.296 | 0.00 | 0.00 | 0.053 | 0.00 | 0.00 |
| ATOM | 1712 | HD23 | LEU | 431 | −6.641 | −0.119 | −7.301 | 0.00 | 0.00 | 0.053 | 0.00 | 0.00 |
| ATOM | 1713 | N | ASN | 432 | −8.776 | 1.630 | −10.845 | 0.00 | 0.00 | −0.650 | 9.00 | −17.40 |
| ATOM | 1714 | HN | ASN | 432 | −8.706 | 2.182 | −9.978 | 0.00 | 0.00 | 0.439 | 0.00 | 0.00 |
| ATOM | 1715 | CA | ASN | 432 | −9.479 | 2.187 | −11.964 | 0.00 | 0.00 | 0.158 | 9.40 | 4.00 |
| ATOM | 1716 | HA | ASN | 432 | −9.819 | 1.315 | −12.523 | 0.00 | 0.00 | 0.053 | 0.00 | 0.00 |
| ATOM | 1717 | C | ASN | 432 | −8.451 | 3.003 | −12.666 | 0.00 | 0.00 | 0.396 | 9.82 | 4.00 |
| ATOM | 1718 | O | ASN | 432 | −7.267 | 2.865 | −12.382 | 0.00 | 0.00 | −0.396 | 8.17 | −17.40 |
| ATOM | 1719 | CB | ASN | 432 | −10.613 | 3.156 | −11.593 | 0.00 | 0.00 | −0.106 | 12.77 | 4.00 |
| ATOM | 1720 | HB1 | ASN | 432 | −10.952 | 3.662 | −12.497 | 0.00 | 0.00 | 0.053 | 0.00 | 0.00 |
| ATOM | 1721 | HB2 | ASN | 432 | −10.232 | 3.883 | −10.876 | 0.00 | 0.00 | 0.053 | 0.00 | 0.00 |
| ATOM | 1722 | CG | ASN | 432 | −11.752 | 2.363 | −10.979 | 0.00 | 0.00 | 0.396 | 9.82 | 4.00 |
| ATOM | 1723 | OD1 | ASN | 432 | −11.654 | 1.151 | −10.791 | 0.00 | 0.00 | −0.396 | 8.17 | −17.40 |
| ATOM | 1724 | ND2 | ASN | 432 | −12.871 | 3.069 | −10.669 | 0.00 | 0.00 | −0.879 | 13.25 | −17.40 |
| ATOM | 1725 | HD21 | ASN | 432 | −12.905 | 4.083 | −10.845 | 0.00 | 0.00 | 0.439 | 0.00 | 0.00 |
| ATOM | 1726 | HD22 | ASN | 432 | −13.685 | 2.590 | −10.258 | 0.00 | 0.00 | 0.439 | 0.00 | 0.00 |
| ATOM | 1727 | N | SER | 433 | −8.850 | 3.869 | −13.615 | 0.00 | 0.00 | −0.650 | 9.00 | −17.40 |
| ATOM | 1728 | HN | SER | 433 | −9.844 | 3.957 | −13.872 | 0.00 | 0.00 | 0.439 | 0.00 | 0.00 |
| ATOM | 1729 | CA | SER | 433 | −7.844 | 4.665 | −14.257 | 0.00 | 0.00 | 0.158 | 9.40 | 4.00 |
| ATOM | 1730 | HA | SER | 433 | −6.992 | 4.740 | −13.582 | 0.00 | 0.00 | 0.053 | 0.00 | 0.00 |
| ATOM | 1731 | C | SER | 433 | −8.427 | 6.014 | −14.531 | 0.00 | 0.00 | 0.396 | 9.82 | 4.00 |
| ATOM | 1732 | O | SER | 433 | −9.642 | 6.196 | −14.494 | 0.00 | 0.00 | −0.396 | 8.17 | −17.40 |
| ATOM | 1733 | CB | SER | 433 | −7.380 | 4.094 | −15.607 | 0.00 | 0.00 | 0.007 | 12.77 | 4.00 |
| ATOM | 1734 | HB1 | SER | 433 | −7.029 | 3.070 | −15.479 | 0.00 | 0.00 | 0.053 | 0.00 | 0.00 |
| ATOM | 1735 | HB2 | SER | 433 | −6.566 | 4.698 | −16.008 | 0.00 | 0.00 | 0.053 | 0.00 | 0.00 |
| ATOM | 1736 | OG | SER | 433 | −8.457 | 4.099 | −16.534 | 0.00 | 0.00 | −0.537 | 11.04 | −17.40 |
| ATOM | 1737 | HG | SER | 433 | −8.714 | 5.071 | −16.757 | 0.00 | 0.00 | 0.424 | 0.00 | 0.00 |
| ATOM | 1738 | N | SER | 434 | −7.549 | 7.001 | −14.805 | 0.00 | 0.00 | −0.650 | 9.00 | −17.40 |
| ATOM | 1739 | HN | SER | 434 | −6.541 | 6.787 | −14.800 | 0.00 | 0.00 | 0.439 | 0.00 | 0.00 |
| ATOM | 1740 | CA | SER | 434 | −7.974 | 8.341 | −15.104 | 0.00 | 0.00 | 0.158 | 9.40 | 4.00 |
| ATOM | 1741 | HA | SER | 434 | −9.061 | 8.314 | −15.179 | 0.00 | 0.00 | 0.053 | 0.00 | 0.00 |
| ATOM | 1742 | C | SER | 434 | −7.328 | 8.716 | −16.400 | 0.00 | 0.00 | 0.396 | 9.82 | 4.00 |
| ATOM | 1743 | O | SER | 434 | −7.362 | 7.952 | −17.362 | 0.00 | 0.00 | −0.396 | 8.17 | −17.40 |
| ATOM | 1744 | CB | SER | 434 | −7.545 | 9.377 | −14.051 | 0.00 | 0.00 | 0.007 | 12.77 | 4.00 |
| ATOM | 1745 | HB1 | SER | 434 | −7.783 | 10.382 | −14.400 | 0.00 | 0.00 | 0.053 | 0.00 | 0.00 |
| ATOM | 1746 | HB2 | SER | 434 | −6.471 | 9.307 | −13.876 | 0.00 | 0.00 | 0.053 | 0.00 | 0.00 |
| ATOM | 1747 | OG | SER | 434 | −8.229 | 9.138 | −12.829 | 0.00 | 0.00 | −0.537 | 11.04 | −17.40 |
| ATOM | 1748 | HG | SER | 434 | −8.558 | 10.033 | −12.438 | 0.00 | 0.00 | 0.424 | 0.00 | 0.00 |
| ATOM | 1749 | N | ASP | 435 | −6.709 | 9.918 | −16.451 | 0.00 | 0.00 | −0.650 | 9.00 | −17.40 |
| ATOM | 1750 | HN | ASP | 435 | −6.730 | 10.496 | −15.598 | 0.00 | 0.00 | 0.439 | 0.00 | 0.00 |
| ATOM | 1751 | CA | ASP | 435 | −6.022 | 10.459 | −17.598 | 0.00 | 0.00 | 0.158 | 9.40 | 4.00 |
| ATOM | 1752 | HA | ASP | 435 | −6.636 | 10.218 | −18.466 | 0.00 | 0.00 | 0.053 | 0.00 | 0.00 |
| ATOM | 1753 | C | ASP | 435 | −4.684 | 9.789 | −17.643 | 0.00 | 0.00 | 0.396 | 9.82 | 4.00 |
| ATOM | 1754 | O | ASP | 435 | −3.700 | 10.361 | −18.108 | 0.00 | 0.00 | −0.396 | 8.17 | −17.40 |
| ATOM | 1755 | CB | ASP | 435 | −5.705 | 11.946 | −17.387 | 0.00 | 0.00 | −0.336 | 12.77 | 4.00 |
| ATOM | 1756 | HB1 | ASP | 435 | −5.225 | 12.302 | −18.299 | 0.00 | 0.00 | 0.053 | 0.00 | 0.00 |
| ATOM | 1757 | HB2 | ASP | 435 | −5.039 | 12.011 | −16.527 | 0.00 | 0.00 | 0.053 | 0.00 | 0.00 |
| ATOM | 1758 | CG | ASP | 435 | −7.023 | 12.648 | −17.133 | 0.00 | 0.00 | 0.297 | 9.82 | 4.00 |
| ATOM | 1759 | CD1 | ASP | 435 | −7.651 | 13.088 | −18.130 | 0.00 | 0.00 | −0.534 | 8.17 | −18.95 |
| ATOM | 1760 | OD2 | ASP | 435 | −7.419 | 12.749 | −15.940 | 0.00 | 0.00 | −0.534 | 8.17 | −18.95 |
| ATOM | 1761 | N | ILE | 436 | −4.644 | 8.537 | −17.167 | 0.00 | 0.00 | −0.650 | 9.00 | −17.40 |
| ATOM | 1762 | HN | ILE | 436 | −5.545 | 8.085 | −16.956 | 0.00 | 0.00 | 0.439 | 0.00 | 0.00 |
| ATOM | 1763 | CA | ILE | 436 | −3.462 | 7.776 | −16.927 | 0.00 | 0.00 | 0.158 | 9.40 | 4.00 |
| ATOM | 1764 | HA | ILE | 436 | −2.796 | 8.366 | −16.297 | 0.00 | 0.00 | 0.053 | 0.00 | 0.00 |
| ATOM | 1765 | C | ILE | 436 | −2.713 | 7.411 | −18.161 | 0.00 | 0.00 | 0.396 | 9.82 | 4.00 |
| ATOM | 1766 | O | ILE | 436 | −1.485 | 7.427 | −18.144 | 0.00 | 0.00 | −0.396 | 8.17 | −17.40 |
| ATOM | 1767 | CB | ILE | 436 | −3.750 | 6.501 | −16.174 | 0.00 | 0.00 | −0.053 | 9.40 | 4.00 |
| ATOM | 1768 | HB | ILE | 436 | −2.798 | 5.984 | −16.051 | 0.00 | 0.00 | 0.053 | 0.00 | 0.00 |
| ATOM | 1769 | CG1 | ILE | 436 | −4.635 | 5.518 | −16.971 | 0.00 | 0.00 | −0.106 | 12.77 | 4.00 |
| ATOM | 1770 | HG11 | ILE | 436 | −5.373 | 6.098 | −17.525 | 0.00 | 0.00 | 0.053 | 0.00 | 0.00 |
| ATOM | 1771 | HG12 | ILE | 436 | −5.123 | 4.849 | −16.262 | 0.00 | 0.00 | 0.053 | 0.00 | 0.00 |
| ATOM | 1772 | CG2 | ILE | 436 | −4.361 | 6.908 | −14.824 | 0.00 | 0.00 | −0.159 | 16.15 | 4.00 |
| ATOM | 1773 | HG21 | ILE | 436 | −4.587 | 6.014 | −14.242 | 0.00 | 0.00 | 0.053 | 0.00 | 0.00 |
| ATOM | 1774 | HG22 | ILE | 436 | −3.652 | 7.528 | −14.276 | 0.00 | 0.00 | 0.053 | 0.00 | 0.00 |
| ATOM | 1775 | HG23 | ILE | 436 | −5.279 | 7.471 | −14.995 | 0.00 | 0.00 | 0.053 | 0.00 | 0.00 |
| ATOM | 1776 | CD1 | ILE | 436 | −3.885 | 4.647 | −17.981 | 0.00 | 0.00 | −0.159 | 16.15 | 4.00 |
| ATOM | 1777 | HD11 | ILE | 436 | −4.591 | 3.991 | −18.490 | 0.00 | 0.00 | 0.053 | 0.00 | 0.00 |
| ATOM | 1778 | HD12 | ILE | 436 | −3.389 | 5.284 | −18.713 | 0.00 | 0.00 | 0.053 | 0.00 | 0.00 |
| ATOM | 1779 | HD13 | ILE | 436 | −3.141 | 4.045 | −17.460 | 0.00 | 0.00 | 0.053 | 0.00 | 0.00 |
| ATOM | 1780 | N | HIS | 437 | −3.407 | 7.102 | −19.267 | 0.00 | 0.00 | −0.650 | 9.00 | −17.40 |
| ATOM | 1781 | HN | HIS | 437 | −4.411 | 7.315 | −19.349 | 0.00 | 0.00 | 0.439 | 0.00 | 0.00 |
| ATOM | 1782 | CA | HIS | 437 | −2.690 | 6.464 | −20.328 | 0.00 | 0.00 | 0.158 | 9.40 | 4.00 |
| ATOM | 1783 | HA | HIS | 437 | −2.295 | 5.520 | −19.952 | 0.00 | 0.00 | 0.053 | 0.00 | 0.00 |
| ATOM | 1784 | C | HIS | 437 | −1.545 | 7.245 | −20.865 | 0.00 | 0.00 | 0.396 | 9.82 | 4.00 |
| ATOM | 1785 | O | HIS | 437 | −1.487 | 8.471 | −20.791 | 0.00 | 0.00 | −0.396 | 8.17 | −17.40 |
| ATOM | 1786 | CB | HIS | 437 | −3.562 | 5.966 | −21.496 | 0.00 | 0.00 | −0.106 | 12.77 | 4.00 |
| ATOM | 1787 | HB1 | HIS | 437 | −2.967 | 5.687 | −22.365 | 0.00 | 0.00 | 0.053 | 0.00 | 0.00 |
| ATOM | 1788 | HB2 | HIS | 437 | −4.270 | 6.723 | −21.834 | 0.00 | 0.00 | 0.053 | 0.00 | 0.00 |
| ATOM | 1789 | CG | HIS | 437 | −4.372 | 4.757 | −21.125 | 0.00 | 0.00 | 0.241 | 7.26 | 0.60 |
| ATOM | 1790 | ND1 | HIS | 437 | −3.821 | 3.506 | −20.957 | 0.00 | 0.00 | −0.481 | 9.25 | −17.40 |
| ATOM | 1791 | CD2 | HIS | 437 | −5.701 | 4.614 | −20.871 | 0.00 | 0.00 | −0.177 | 10.80 | 0.60 |
| ATOM | 1792 | HD2 | HIS | 437 | −6.433 | 5.420 | −20.920 | 0.00 | 0.00 | 0.127 | 0.00 | 0.00 |
| ATOM | 1793 | CE1 | HIS | 437 | −4.834 | 2.676 | −20.606 | 0.00 | 0.00 | 0.064 | 10.80 | 0.60 |
| ATOM | 1794 | HE1 | HIS | 437 | −4.705 | 1.613 | −20.401 | 0.00 | 0.00 | 0.127 | 0.00 | 0.00 |
| ATOM | 1795 | NE2 | HIS | 437 | −5.995 | 3.302 | −20.541 | 0.00 | 0.00 | −0.292 | 9.25 | −17.40 |
| ATOM | 1796 | HE2 | HIS | 437 | −6.913 | 2.903 | −20.299 | 0.00 | 0.00 | 0.393 | 0.00 | 0.00 |
| ATOM | 1797 | N | HIS | 438 | −0.531 | 6.475 | −21.307 | 0.00 | 0.00 | −0.650 | 9.00 | −17.40 |
| ATOM | 1798 | HN | HIS | 438 | −0.577 | 5.462 | −21.128 | 0.00 | 0.00 | 0.439 | 0.00 | 0.00 |
| ATOM | 1799 | CA | HIS | 438 | 0.603 | 6.982 | −22.009 | 0.00 | 0.00 | 0.158 | 9.40 | 4.00 |
| ATOM | 1800 | HA | HIS | 438 | 0.962 | 7.906 | −21.555 | 0.00 | 0.00 | 0.053 | 0.00 | 0.00 |
| ATOM | 1801 | C | HIS | 438 | 0.090 | 7.211 | −23.388 | 0.00 | 0.00 | 0.396 | 9.82 | 4.00 |
| ATOM | 1802 | O | HIS | 438 | 0.412 | 8.198 | −24.041 | 0.00 | 0.00 | −0.396 | 8.17 | −17.40 |
| ATOM | 1803 | CB | HIS | 438 | 1.749 | 5.962 | −22.085 | 0.00 | 0.00 | −0.106 | 12.77 | 4.00 |
| ATOM | 1804 | HB1 | HIS | 438 | 1.380 | 5.078 | −22.605 | 0.00 | 0.00 | 0.053 | 0.00 | 0.00 |
| ATOM | 1805 | HB2 | HIS | 438 | 2.050 | 5.716 | −21.067 | 0.00 | 0.00 | 0.053 | 0.00 | 0.00 |
| ATOM | 1806 | CG | HIS | 438 | 2.975 | 6.426 | −22.811 | 0.00 | 0.00 | 0.241 | 7.26 | 0.60 |
| ATOM | 1807 | ND1 | HIS | 438 | 4.151 | 6.805 | −22.199 | 0.00 | 0.00 | −0.481 | 9.25 | −17.40 |
| ATOM | 1808 | CD2 | HIS | 438 | 3.201 | 6.538 | −24.147 | 0.00 | 0.00 | −0.177 | 10.80 | 0.60 |
| ATOM | 1809 | HD2 | HIS | 438 | 2.467 | 6.314 | −24.921 | 0.00 | 0.00 | 0.127 | 0.00 | 0.00 |
| ATOM | 1810 | CE1 | HIS | 438 | 5.021 | 7.125 | −23.190 | 0.00 | 0.00 | 0.064 | 10.80 | 0.60 |
| ATOM | 1811 | HE1 | HIS | 438 | 6.041 | 7.467 | −23.016 | 0.00 | 0.00 | 0.127 | 0.00 | 0.00 |
| ATOM | 1812 | NE2 | HIS | 438 | 4.490 | 6.975 | −24.389 | 0.00 | 0.00 | −0.292 | 9.25 | −17.40 |
| ATOM | 1813 | HE2 | HIS | 438 | 4.936 | 7.146 | −25.301 | 0.00 | 0.00 | 0.393 | 0.00 | 0.00 |
| ATOM | 1814 | N | ILE | 439 | −0.757 | 6.283 | −23.870 | 0.00 | 0.00 | −0.650 | 9.00 | −17.40 |
| ATOM | 1815 | HN | ILE | 439 | −0.980 | 5.434 | −23.330 | 0.00 | 0.00 | 0.439 | 0.00 | 0.00 |
| ATOM | 1816 | CA | ILE | 439 | −1.336 | 6.513 | −25.152 | 0.00 | 0.00 | 0.158 | 9.40 | 4.00 |
| ATOM | 1817 | HA | ILE | 439 | −0.567 | 6.655 | −25.912 | 0.00 | 0.00 | 0.053 | 0.00 | 0.00 |
| ATOM | 1818 | C | ILE | 439 | −2.138 | 7.745 | −24.933 | 0.00 | 0.00 | 0.396 | 9.82 | 4.00 |
| ATOM | 1819 | O | ILE | 439 | −2.254 | 8.603 | −25.807 | 0.00 | 0.00 | −0.396 | 8.17 | −17.40 |
| ATOM | 1820 | CB | ILE | 439 | −2.289 | 5.445 | −25.604 | 0.00 | 0.00 | −0.053 | 9.40 | 4.00 |
| ATOM | 1821 | HB | ILE | 439 | −3.101 | 5.446 | −24.877 | 0.00 | 0.00 | 0.053 | 0.00 | 0.00 |
| ATOM | 1822 | CG1 | ILE | 439 | −1.629 | 4.054 | −25.598 | 0.00 | 0.00 | −0.106 | 12.77 | 4.00 |
| ATOM | 1823 | HG11 | ILE | 439 | −0.703 | 4.113 | −26.170 | 0.00 | 0.00 | 0.053 | 0.00 | 0.00 |
| ATOM | 1824 | HG12 | ILE | 439 | −2.320 | 3.346 | −26.056 | 0.00 | 0.00 | 0.053 | 0.00 | 0.00 |
| ATOM | 1825 | CG2 | ILE | 439 | −2.745 | 5.862 | −27.011 | 0.00 | 0.00 | −0.159 | 16.15 | 4.00 |
| ATOM | 1826 | HG21 | ILE | 439 | −3.445 | 5.123 | −27.401 | 0.00 | 0.00 | 0.053 | 0.00 | 0.00 |
| ATOM | 1827 | HG22 | ILE | 439 | −3.235 | 6.834 | −26.961 | 0.00 | 0.00 | 0.053 | 0.00 | 0.00 |
| ATOM | 1828 | HG23 | ILE | 439 | −1.879 | 5.925 | −27.670 | 0.00 | 0.00 | 0.053 | 0.00 | 0.00 |
| ATOM | 1829 | CD1 | ILE | 439 | −1.280 | 3.524 | −24.208 | 0.00 | 0.00 | −0.159 | 16.15 | 4.00 |
| ATOM | 1830 | HD11 | ILE | 439 | −0.820 | 2.540 | −24.299 | 0.00 | 0.00 | 0.053 | 0.00 | 0.00 |
| ATOM | 1831 | HD12 | ILE | 439 | −0.583 | 4.207 | −23.723 | 0.00 | 0.00 | 0.053 | 0.00 | 0.00 |
| ATOM | 1832 | HD13 | ILE | 439 | −2.188 | 3.446 | −23.610 | 0.00 | 0.00 | 0.053 | 0.00 | 0.00 |
| ATOM | 1833 | N | ASN | 440 | −2.698 | 7.844 | −23.713 | 0.00 | 0.00 | −0.650 | 9.00 | −17.40 |
| ATOM | 1834 | HN | ASN | 440 | −2.585 | 7.066 | −23.047 | 0.00 | 0.00 | 0.439 | 0.00 | 0.00 |
| ATOM | 1835 | CA | ASN | 440 | −3.443 | 8.992 | −23.314 | 0.00 | 0.00 | 0.158 | 9.40 | 4.00 |
| ATOM | 1836 | HA | ASN | 440 | −3.988 | 9.317 | −24.200 | 0.00 | 0.00 | 0.053 | 0.00 | 0.00 |
| ATOM | 1837 | C | ASN | 440 | −2.435 | 9.994 | −22.854 | 0.00 | 0.00 | 0.396 | 9.82 | 4.00 |
| ATOM | 1838 | O | ASN | 440 | −1.247 | 9.894 | −23.155 | 0.00 | 0.00 | −0.396 | 8.17 | −17.40 |
| ATOM | 1839 | CB | ASN | 440 | −4.419 | 8.716 | −22.155 | 0.00 | 0.00 | −0.106 | 12.77 | 4.00 |
| ATOM | 1840 | HB1 | ASN | 440 | −3.886 | 8.706 | −21.204 | 0.00 | 0.00 | 0.053 | 0.00 | 0.00 |
| ATOM | 1841 | HB2 | ASN | 440 | −4.905 | 7.750 | −22.290 | 0.00 | 0.00 | 0.053 | 0.00 | 0.00 |
| ATOM | 1842 | CG | ASN | 440 | −5.496 | 9.799 | −22.097 | 0.00 | 0.00 | 0.396 | 9.82 | 4.00 |
| ATOM | 1843 | OD1 | ASN | 440 | −6.254 | 9.866 | −21.129 | 0.00 | 0.00 | −0.396 | 8.17 | −17.40 |
| ATOM | 1844 | ND2 | ASN | 440 | −5.579 | 10.657 | −23.149 | 0.00 | 0.00 | −0.879 | 13.25 | −17.40 |
| ATOM | 1845 | HD21 | ASN | 440 | −4.925 | 10.567 | −23.940 | 0.00 | 0.00 | 0.439 | 0.00 | 0.00 |
| ATOM | 1846 | HD22 | ASN | 440 | −6.296 | 11.397 | −23.155 | 0.00 | 0.00 | 0.439 | 0.00 | 0.00 |
| ATOM | 1847 | N | GLU | 441 | −2.904 | 11.004 | −22.114 | 0.00 | 0.00 | −0.650 | 9.00 | −17.40 |
| ATOM | 1848 | HN | GLU | 441 | −3.888 | 10.976 | −21.811 | 0.00 | 0.00 | 0.439 | 0.00 | 0.00 |
| ATOM | 1849 | CA | GLU | 441 | −2.099 | 12.125 | −21.723 | 0.00 | 0.00 | 0.158 | 9.40 | 4.00 |
| ATOM | 1850 | HA | GLU | 441 | −1.602 | 12.499 | −22.618 | 0.00 | 0.00 | 0.053 | 0.00 | 0.00 |
| ATOM | 1851 | C | GLU | 441 | −1.014 | 11.887 | −20.700 | 0.00 | 0.00 | 0.396 | 9.82 | 4.00 |
| ATOM | 1852 | O | GLU | 441 | 0.153 | 12.158 | −20.978 | 0.00 | 0.00 | −0.396 | 8.17 | −17.40 |
| ATOM | 1853 | CB | GLU | 441 | −3.001 | 13.248 | −21.188 | 0.00 | 0.00 | −0.106 | 12.77 | 4.00 |
| ATOM | 1854 | HB1 | GLU | 441 | −3.744 | 13.561 | −21.921 | 0.00 | 0.00 | 0.053 | 0.00 | 0.00 |
| ATOM | 1855 | HB2 | GLU | 441 | −2.432 | 14.138 | −20.919 | 0.00 | 0.00 | 0.053 | 0.00 | 0.00 |
| ATOM | 1856 | CG | GLU | 441 | −3.770 | 12.821 | −19.936 | 0.00 | 0.00 | −0.336 | 12.77 | 4.00 |
| ATOM | 1857 | HG1 | GLU | 441 | −3.218 | 13.176 | −19.066 | 0.00 | 0.00 | 0.053 | 0.00 | 0.00 |
| ATOM | 1858 | HG2 | GLU | 441 | −3.837 | 11.733 | −19.938 | 0.00 | 0.00 | 0.053 | 0.00 | 0.00 |
| ATOM | 1859 | CD | GLU | 441 | −5.151 | 13.451 | −19.998 | 0.00 | 0.00 | 0.297 | 9.82 | 4.00 |
| ATOM | 1860 | OE1 | GLU | 441 | −5.889 | 13.099 | −20.956 | 0.00 | 0.00 | −0.534 | 8.17 | −18.95 |
| ATOM | 1861 | OE2 | GLU | 441 | −5.495 | 14.269 | −19.103 | 0.00 | 0.00 | −0.534 | 8.17 | −18.95 |
| ATOM | 1862 | N | ILE | 442 | −1.343 | 11.353 | −19.503 | 0.00 | 0.00 | −0.650 | 9.00 | −17.40 |
| ATOM | 1863 | HN | ILE | 442 | −2.258 | 10.902 | −19.363 | 0.00 | 0.00 | 0.439 | 0.00 | 0.00 |
| ATOM | 1864 | CA | ILE | 442 | −0.384 | 11.427 | −18.422 | 0.00 | 0.00 | 0.158 | 9.40 | 4.00 |
| ATOM | 1865 | HA | ILE | 442 | −0.084 | 12.470 | −18.316 | 0.00 | 0.00 | 0.053 | 0.00 | 0.00 |
| ATOM | 1866 | C | ILE | 442 | 0.870 | 10.631 | −18.583 | 0.00 | 0.00 | 0.396 | 9.82 | 4.00 |
| ATOM | 1867 | O | ILE | 442 | 1.965 | 11.191 | −18.522 | 0.00 | 0.00 | −0.396 | 8.17 | −17.40 |
| ATOM | 1868 | CB | ILE | 442 | −0.942 | 11.031 | −17.085 | 0.00 | 0.00 | −0.053 | 9.40 | 4.00 |
| ATOM | 1869 | HB | ILE | 442 | −1.416 | 10.057 | −17.206 | 0.00 | 0.00 | 0.053 | 0.00 | 0.00 |
| ATOM | 1870 | CG1 | ILE | 442 | −2.054 | 11.992 | −16.638 | 0.00 | 0.00 | −0.106 | 12.77 | 4.00 |
| ATOM | 1871 | HG11 | ILE | 442 | −2.821 | 12.146 | −17.397 | 0.00 | 0.00 | 0.053 | 0.00 | 0.00 |
| ATOM | 1872 | HG12 | ILE | 442 | −1.688 | 12.988 | −16.390 | 0.00 | 0.00 | 0.053 | 0.00 | 0.00 |
| ATOM | 1873 | CG2 | ILE | 442 | 0.235 | 10.966 | −16.097 | 0.00 | 0.00 | −0.159 | 16.15 | 4.00 |
| ATOM | 1874 | HG21 | ILE | 442 | −0.133 | 10.680 | −15.111 | 0.00 | 0.00 | 0.053 | 0.00 | 0.00 |
| ATOM | 1875 | HG22 | ILE | 442 | 0.959 | 10.228 | −16.442 | 0.00 | 0.00 | 0.053 | 0.00 | 0.00 |
| ATOM | 1876 | HG23 | ILE | 442 | 0.714 | 11.943 | −16.036 | 0.00 | 0.00 | 0.053 | 0.00 | 0.00 |
| ATOM | 1877 | CD1 | ILE | 442 | −2.803 | 11.513 | −15.394 | 0.00 | 0.00 | −0.159 | 16.15 | 4.00 |
| ATOM | 1878 | HD11 | ILE | 442 | −3.574 | 12.237 | −15.132 | 0.00 | 0.00 | 0.053 | 0.00 | 0.00 |
| ATOM | 1879 | HD12 | ILE | 442 | −3.266 | 10.547 | −15.597 | 0.00 | 0.00 | 0.053 | 0.00 | 0.00 |
| ATOM | 1880 | HD13 | ILE | 442 | −2.103 | 11.412 | −14.564 | 0.00 | 0.00 | 0.053 | 0.00 | 0.00 |
| ATOM | 1881 | N | LYS | 443 | 0.767 | 9.311 | −18.809 | 0.00 | 0.00 | −0.650 | 9.00 | −17.40 |
| ATOM | 1882 | HN | LYS | 443 | −0.138 | 8.869 | −19.026 | 0.00 | 0.00 | 0.439 | 0.00 | 0.00 |
| ATOM | 1883 | CA | LYS | 443 | 1.978 | 8.548 | −18.734 | 0.00 | 0.00 | 0.158 | 9.40 | 4.00 |
| ATOM | 1884 | HA | LYS | 443 | 2.491 | 8.698 | −17.784 | 0.00 | 0.00 | 0.053 | 0.00 | 0.00 |
| ATOM | 1885 | C | LYS | 443 | 2.933 | 8.923 | −19.813 | 0.00 | 0.00 | 0.396 | 9.82 | 4.00 |
| ATOM | 1886 | O | LYS | 443 | 4.137 | 8.994 | −19.578 | 0.00 | 0.00 | −0.396 | 8.17 | −17.40 |
| ATOM | 1887 | CB | LYS | 443 | 1.780 | 7.027 | −18.740 | 0.00 | 0.00 | −0.106 | 12.77 | 4.00 |
| ATOM | 1888 | HB1 | LYS | 443 | 1.427 | 6.682 | −19.712 | 0.00 | 0.00 | 0.053 | 0.00 | 0.00 |
| ATOM | 1889 | HB2 | LYS | 443 | 1.047 | 6.725 | −17.992 | 0.00 | 0.00 | 0.053 | 0.00 | 0.00 |
| ATOM | 1890 | CG | LYS | 443 | 3.090 | 6.302 | −18.433 | 0.00 | 0.00 | −0.106 | 12.77 | 4.00 |
| ATOM | 1891 | HG1 | LYS | 443 | 3.760 | 6.886 | −17.803 | 0.00 | 0.00 | 0.053 | 0.00 | 0.00 |
| ATOM | 1892 | HG2 | LYS | 443 | 3.661 | 6.060 | −19.330 | 0.00 | 0.00 | 0.053 | 0.00 | 0.00 |
| ATOM | 1893 | CD | LYS | 443 | 2.879 | 4.979 | −17.703 | 0.00 | 0.00 | −0.106 | 12.77 | 4.00 |
| ATOM | 1894 | HD1 | LYS | 443 | 3.828 | 4.493 | −17.478 | 0.00 | 0.00 | 0.053 | 0.00 | 0.00 |
| ATOM | 1895 | HD2 | LYS | 443 | 2.289 | 4.284 | −18.301 | 0.00 | 0.00 | 0.053 | 0.00 | 0.00 |
| ATOM | 1896 | CE | LYS | 443 | 2.142 | 5.184 | −16.376 | 0.00 | 0.00 | 0.099 | 12.77 | 4.00 |
| ATOM | 1897 | HE1 | LYS | 443 | 2.337 | 4.344 | −15.709 | 0.00 | 0.00 | 0.053 | 0.00 | 0.00 |
| ATOM | 1898 | HE2 | LYS | 443 | 1.069 | 5.254 | −16.554 | 0.00 | 0.00 | 0.053 | 0.00 | 0.00 |
| ATOM | 1899 | NZ | LYS | 443 | 2.607 | 6.434 | −15.727 | 0.00 | 0.00 | −0.045 | 13.25 | −39.20 |
| ATOM | 1900 | HZ1 | LYS | 443 | 2.107 | 6.565 | −14.836 | 0.00 | 0.00 | 0.280 | 0.00 | 0.00 |
| ATOM | 1901 | HZ2 | LYS | 443 | 3.619 | 6.372 | −15.547 | 0.00 | 0.00 | 0.280 | 0.00 | 0.00 |
| ATOM | 1902 | HZ3 | LYS | 443 | 2.418 | 7.234 | −16.347 | 0.00 | 0.00 | 0.280 | 0.00 | 0.00 |
| ATOM | 1903 | N | ARG | 444 | 2.437 | 9.161 | −21.035 | 0.00 | 0.00 | −0.650 | 9.00 | −17.40 |
| ATOM | 1904 | HN | ARG | 444 | 1.428 | 9.078 | −21.222 | 0.00 | 0.00 | 0.439 | 0.00 | 0.00 |
| ATOM | 1905 | CA | ARG | 444 | 3.349 | 9.533 | −22.072 | 0.00 | 0.00 | 0.158 | 9.40 | 4.00 |
| ATOM | 1906 | HA | ARG | 444 | 4.146 | 8.790 | −22.093 | 0.00 | 0.00 | 0.053 | 0.00 | 0.00 |
| ATOM | 1907 | C | ARG | 444 | 3.882 | 10.889 | −21.742 | 0.00 | 0.00 | 0.396 | 9.82 | 4.00 |
| ATOM | 1908 | O | ARG | 444 | 4.992 | 11.246 | −22.140 | 0.00 | 0.00 | −0.396 | 8.17 | −17.40 |
| ATOM | 1909 | CB | ARG | 444 | 2.720 | 9.503 | −23.463 | 0.00 | 0.00 | −0.106 | 12.77 | 4.00 |
| ATOM | 1910 | HB1 | ARG | 444 | 2.006 | 10.325 | −23.522 | 0.00 | 0.00 | 0.053 | 0.00 | 0.00 |
| ATOM | 1911 | HB2 | ARG | 444 | 2.223 | 8.540 | −23.581 | 0.00 | 0.00 | 0.053 | 0.00 | 0.00 |
| ATOM | 1912 | CG | ARG | 444 | 3.671 | 9.658 | −24.651 | 0.00 | 0.00 | −0.106 | 12.77 | 4.00 |
| ATOM | 1913 | HG1 | ARG | 444 | 4.241 | 8.740 | −24.795 | 0.00 | 0.00 | 0.053 | 0.00 | 0.00 |
| ATOM | 1914 | HG2 | ARG | 444 | 4.364 | 10.479 | −24.469 | 0.00 | 0.00 | 0.053 | 0.00 | 0.00 |
| ATOM | 1915 | CD | ARG | 444 | 2.874 | 9.953 | −25.925 | 0.00 | 0.00 | 0.374 | 12.77 | 4.00 |
| ATOM | 1916 | HD1 | ARG | 444 | 2.078 | 10.656 | −25.680 | 0.00 | 0.00 | 0.053 | 0.00 | 0.00 |
| ATOM | 1917 | HD2 | ARG | 444 | 2.455 | 9.017 | −26.295 | 0.00 | 0.00 | 0.053 | 0.00 | 0.00 |
| ATOM | 1918 | NE | ARG | 444 | 3.795 | 10.543 | −26.931 | 0.00 | 0.00 | −0.819 | 9.00 | −24.67 |
| ATOM | 1919 | HE | ARG | 444 | 4.292 | 11.424 | −26.739 | 0.00 | 0.00 | 0.407 | 0.00 | 0.00 |
| ATOM | 1920 | CZ | ARG | 444 | 3.965 | 9.890 | −28.111 | 0.00 | 0.00 | 0.796 | 6.95 | 4.00 |
| ATOM | 1921 | NH1 | ARG | 444 | 3.315 | 8.704 | −28.296 | 0.00 | 0.00 | −0.746 | 9.00 | −24.67 |
| ATOM | 1922 | HH11 | ARG | 444 | 3.426 | 8.188 | −29.181 | 0.00 | 0.00 | 0.407 | 0.00 | 0.00 |
| ATOM | 1923 | HH12 | ARG | 444 | 2.713 | 8.325 | −27.551 | 0.00 | 0.00 | 0.407 | 0.00 | 0.00 |
| ATOM | 1924 | NH2 | ARG | 444 | 4.786 | 10.403 | −29.075 | 0.00 | 0.00 | −0.746 | 9.00 | −24.67 |
| ATOM | 1925 | HH21 | ARG | 444 | 4.909 | 9.902 | −29.966 | 0.00 | 0.00 | 0.407 | 0.00 | 0.00 |
| ATOM | 1926 | HH22 | ARG | 444 | 5.281 | 11.291 | −28.910 | 0.00 | 0.00 | 0.407 | 0.00 | 0.00 |
| ATOM | 1927 | N | GLN | 445 | 3.085 | 11.690 | −21.006 | 0.00 | 0.00 | −0.650 | 9.00 | −17.40 |
| ATOM | 1928 | HN | GLN | 445 | 2.149 | 11.367 | −20.722 | 0.00 | 0.00 | 0.439 | 0.00 | 0.00 |
| ATOM | 1929 | CA | GLN | 445 | 3.542 | 12.989 | −20.620 | 0.00 | 0.00 | 0.158 | 9.40 | 4.00 |
| ATOM | 1930 | HA | GLN | 445 | 3.674 | 13.576 | −21.529 | 0.00 | 0.00 | 0.053 | 0.00 | 0.00 |
| ATOM | 1931 | C | GLN | 445 | 4.835 | 12.816 | −19.895 | 0.00 | 0.00 | 0.396 | 9.82 | 4.00 |
| ATOM | 1932 | O | GLN | 445 | 5.880 | 13.141 | −20.453 | 0.00 | 0.00 | −0.396 | 8.17 | −17.40 |
| ATOM | 1933 | CB | GLN | 445 | 2.557 | 13.669 | −19.651 | 0.00 | 0.00 | −0.106 | 12.77 | 4.00 |
| ATOM | 1934 | HB1 | GLN | 445 | 2.453 | 13.037 | −18.769 | 0.00 | 0.00 | 0.053 | 0.00 | 0.00 |
| ATOM | 1935 | HB2 | GLN | 445 | 1.599 | 13.780 | −20.159 | 0.00 | 0.00 | 0.053 | 0.00 | 0.00 |
| ATOM | 1936 | CG | GLN | 445 | 2.996 | 15.053 | −19.180 | 0.00 | 0.00 | −0.106 | 12.77 | 4.00 |
| ATOM | 1937 | HG1 | GLN | 445 | 3.101 | 15.675 | −20.069 | 0.00 | 0.00 | 0.053 | 0.00 | 0.00 |
| ATOM | 1938 | HG2 | GLN | 445 | 3.946 | 14.927 | −18.660 | 0.00 | 0.00 | 0.053 | 0.00 | 0.00 |
| ATOM | 1939 | CD | GLN | 445 | 1.910 | 15.573 | −18.253 | 0.00 | 0.00 | 0.396 | 9.82 | 4.00 |
| ATOM | 1940 | OE1 | GLN | 445 | 1.714 | 15.071 | −17.145 | 0.00 | 0.00 | −0.396 | 8.17 | −17.40 |
| ATOM | 1941 | NE2 | GLN | 445 | 1.165 | 16.604 | −18.729 | 0.00 | 0.00 | −0.879 | 13.25 | −17.40 |
| ATOM | 1942 | HE21 | GLN | 445 | 1.362 | 16.995 | −19.661 | 0.00 | 0.00 | 0.439 | 0.00 | 0.00 |
| ATOM | 1943 | HE22 | GLN | 445 | 0.403 | 16.995 | −18.158 | 0.00 | 0.00 | 0.439 | 0.00 | 0.00 |
| ATOM | 1944 | N | ASN | 446 | 4.778 | 12.157 | −18.714 | 0.00 | 0.00 | −0.650 | 9.00 | −17.40 |
| ATOM | 1945 | HN | ASN | 446 | 3.875 | 11.724 | −18.472 | 0.00 | 0.00 | 0.439 | 0.00 | 0.00 |
| ATOM | 1946 | CA | ASN | 446 | 5.848 | 11.996 | −17.758 | 0.00 | 0.00 | 0.158 | 9.40 | 4.00 |
| ATOM | 1947 | HA | ASN | 446 | 6.285 | 12.957 | −17.488 | 0.00 | 0.00 | 0.053 | 0.00 | 0.00 |
| ATOM | 1948 | C | ASN | 446 | 6.943 | 11.140 | −18.308 | 0.00 | 0.00 | 0.396 | 9.82 | 4.00 |
| ATOM | 1949 | O | ASN | 446 | 8.065 | 11.181 | −17.803 | 0.00 | 0.00 | −0.396 | 8.17 | −17.40 |
| ATOM | 1950 | CB | ASN | 446 | 5.430 | 11.333 | −16.430 | 0.00 | 0.00 | −0.106 | 12.77 | 4.00 |
| ATOM | 1951 | HB1 | ASN | 446 | 4.466 | 11.732 | −16.114 | 0.00 | 0.00 | 0.053 | 0.00 | 0.00 |
| ATOM | 1952 | HB2 | ASN | 446 | 6.180 | 11.545 | −15.668 | 0.00 | 0.00 | 0.053 | 0.00 | 0.00 |
| ATOM | 1953 | CG | ASN | 446 | 5.318 | 9.825 | −16.630 | 0.00 | 0.00 | 0.396 | 9.82 | 4.00 |
| ATOM | 1954 | OD1 | ASN | 446 | 4.295 | 9.281 | −17.043 | 0.00 | 0.00 | −0.396 | 8.17 | −17.40 |
| ATOM | 1955 | ND2 | ASN | 446 | 6.433 | 9.110 | −16.319 | 0.00 | 0.00 | −0.879 | 13.25 | −17.40 |
| ATOM | 1956 | HD21 | ASN | 446 | 7.275 | 9.593 | −15.975 | 0.00 | 0.00 | 0.439 | 0.00 | 0.00 |
| ATOM | 1957 | HD22 | ASN | 446 | 6.435 | 8.086 | −16.427 | 0.00 | 0.00 | 0.439 | 0.00 | 0.00 |
| ATOM | 1958 | N | GLN | 447 | 6.639 | 10.336 | −19.343 | 0.00 | 0.00 | −0.650 | 9.00 | −17.40 |
| ATOM | 1959 | HN | GLN | 447 | 5.690 | 10.386 | −19.739 | 0.00 | 0.00 | 0.439 | 0.00 | 0.00 |
| ATOM | 1960 | CA | GLN | 447 | 7.571 | 9.414 | −19.921 | 0.00 | 0.00 | 0.158 | 9.40 | 4.00 |
| ATOM | 1961 | HA | GLN | 447 | 7.753 | 8.620 | −19.196 | 0.00 | 0.00 | 0.053 | 0.00 | 0.00 |
| ATOM | 1962 | C | GLN | 447 | 8.827 | 10.157 | −20.222 | 0.00 | 0.00 | 0.396 | 9.82 | 4.00 |
| ATOM | 1963 | O | GLN | 447 | 9.915 | 9.620 | −20.067 | 0.00 | 0.00 | −0.396 | 8.17 | −17.40 |
| ATOM | 1964 | CB | GLN | 447 | 7.050 | 8.836 | −21.252 | 0.00 | 0.00 | −0.106 | 12.77 | 4.00 |
| ATOM | 1965 | HB1 | GLN | 447 | 6.787 | 9.669 | −21.904 | 0.00 | 0.00 | 0.053 | 0.00 | 0.00 |
| ATOM | 1966 | HB2 | GLN | 447 | 6.175 | 8.223 | −21.037 | 0.00 | 0.00 | 0.053 | 0.00 | 0.00 |
| ATOM | 1967 | CG | GLN | 447 | 8.052 | 7.957 | −22.009 | 0.00 | 0.00 | −0.106 | 12.77 | 4.00 |
| ATOM | 1968 | HG1 | GLN | 447 | 7.537 | 7.109 | −22.460 | 0.00 | 0.00 | 0.053 | 0.00 | 0.00 |
| ATOM | 1969 | HG2 | GLN | 447 | 8.813 | 7.585 | −21.323 | 0.00 | 0.00 | 0.053 | 0.00 | 0.00 |
| ATOM | 1970 | CD | GLN | 447 | 8.731 | 8.771 | −23.113 | 0.00 | 0.00 | 0.396 | 9.82 | 4.00 |
| ATOM | 1971 | OE1 | GLN | 447 | 9.219 | 9.883 | −22.915 | 0.00 | 0.00 | −0.396 | 8.17 | −17.40 |
| ATOM | 1972 | NE2 | GLN | 447 | 8.761 | 8.189 | −24.341 | 0.00 | 0.00 | −0.879 | 13.25 | −17.40 |
| ATOM | 1973 | HE21 | GLN | 447 | 8.345 | 7.257 | −24.479 | 0.00 | 0.00 | 0.439 | 0.00 | 0.00 |
| ATOM | 1974 | HE22 | GLN | 447 | 9.200 | 8.680 | −25.133 | 0.00 | 0.00 | 0.439 | 0.00 | 0.00 |
| ATOM | 1975 | N | SER | 448 | 8.743 | 11.419 | −20.664 | 0.00 | 0.00 | −0.650 | 9.00 | −17.40 |
| ATOM | 1976 | HN | SER | 448 | 7.832 | 11.887 | −20.776 | 0.00 | 0.00 | 0.439 | 0.00 | 0.00 |
| ATOM | 1977 | CA | SER | 448 | 9.967 | 12.093 | −20.975 | 0.00 | 0.00 | 0.158 | 9.40 | 4.00 |
| ATOM | 1978 | HA | SER | 448 | 10.522 | 11.537 | −21.731 | 0.00 | 0.00 | 0.053 | 0.00 | 0.00 |
| ATOM | 1979 | C | SER | 448 | 10.820 | 12.215 | −19.744 | 0.00 | 0.00 | 0.396 | 9.82 | 4.00 |
| ATOM | 1980 | O | SER | 448 | 12.033 | 12.030 | −19.814 | 0.00 | 0.00 | −0.396 | 8.17 | −17.40 |
| ATOM | 1981 | CB | SER | 448 | 9.747 | 13.501 | −21.554 | 0.00 | 0.00 | 0.007 | 12.77 | 4.00 |
| ATOM | 1982 | HB1 | SER | 448 | 10.705 | 14.010 | −21.660 | 0.00 | 0.00 | 0.053 | 0.00 | 0.00 |
| ATOM | 1983 | HB2 | SER | 448 | 9.107 | 14.078 | −20.887 | 0.00 | 0.00 | 0.053 | 0.00 | 0.00 |
| ATOM | 1984 | OG | SER | 448 | 9.127 | 13.405 | −22.828 | 0.00 | 0.00 | −0.537 | 11.04 | −17.40 |
| ATOM | 1985 | HG | SER | 448 | 8.911 | 14.350 | −23.177 | 0.00 | 0.00 | 0.424 | 0.00 | 0.00 |
| ATOM | 1986 | N | LEU | 449 | 10.231 | 12.546 | −18.578 | 0.00 | 0.00 | −0.650 | 9.00 | −17.40 |
| ATOM | 1987 | HN | LEU | 449 | 9.212 | 12.686 | −18.521 | 0.00 | 0.00 | 0.439 | 0.00 | 0.00 |
| ATOM | 1988 | CA | LEU | 449 | 11.062 | 12.698 | −17.416 | 0.00 | 0.00 | 0.158 | 9.40 | 4.00 |
| ATOM | 1989 | HA | LEU | 449 | 11.854 | 13.426 | −17.594 | 0.00 | 0.00 | 0.053 | 0.00 | 0.00 |
| ATOM | 1990 | C | LEU | 449 | 11.708 | 11.397 | −17.042 | 0.00 | 0.00 | 0.396 | 9.82 | 4.00 |
| ATOM | 1991 | O | LEU | 449 | 12.931 | 11.322 | −16.932 | 0.00 | 0.00 | −0.396 | 8.17 | −17.40 |
| ATOM | 1992 | CB | LEU | 449 | 10.291 | 13.206 | −16.182 | 0.00 | 0.00 | −0.106 | 12.77 | 4.00 |
| ATOM | 1993 | HB1 | LEU | 449 | 9.479 | 12.510 | −15.974 | 0.00 | 0.00 | 0.053 | 0.00 | 0.00 |
| ATOM | 1994 | HB2 | LEU | 449 | 9.897 | 14.197 | −16.407 | 0.00 | 0.00 | 0.053 | 0.00 | 0.00 |
| ATOM | 1995 | CG | LEU | 449 | 11.151 | 13.320 | −14.907 | 0.00 | 0.00 | −0.053 | 9.40 | 4.00 |
| ATOM | 1996 | HG | LEU | 449 | 11.542 | 12.351 | −14.596 | 0.00 | 0.00 | 0.053 | 0.00 | 0.00 |
| ATOM | 1997 | CD1 | LEU | 449 | 12.357 | 14.249 | −15.115 | 0.00 | 0.00 | −0.159 | 16.15 | 4.00 |
| ATOM | 1998 | HD11 | LEU | 449 | 12.938 | 14.303 | −14.194 | 0.00 | 0.00 | 0.053 | 0.00 | 0.00 |
| ATOM | 1999 | HD12 | LEU | 449 | 12.983 | 13.858 | −15.917 | 0.00 | 0.00 | 0.053 | 0.00 | 0.00 |
| ATOM | 2000 | HD13 | LEU | 449 | 12.007 | 15.246 | −15.382 | 0.00 | 0.00 | 0.053 | 0.00 | 0.00 |
| ATOM | 2001 | CD2 | LEU | 449 | 10.297 | 13.742 | −13.701 | 0.00 | 0.00 | −0.159 | 16.15 | 4.00 |
| ATOM | 2002 | HD21 | LEU | 449 | 10.929 | 13.815 | −12.816 | 0.00 | 0.00 | 0.053 | 0.00 | 0.00 |
| ATOM | 2003 | HD22 | LEU | 449 | 9.839 | 14.711 | −13.901 | 0.00 | 0.00 | 0.053 | 0.00 | 0.00 |
| ATOM | 2004 | HD23 | LEU | 449 | 9.517 | 13.000 | −13.530 | 0.00 | 0.00 | 0.053 | 0.00 | 0.00 |
| ATOM | 2005 | N | VAL | 450 | 10.905 | 10.331 | −16.843 | 0.00 | 0.00 | −0.650 | 9.00 | −17.40 |
| ATOM | 2006 | HN | VAL | 450 | 9.891 | 10.418 | −17.000 | 0.00 | 0.00 | 0.439 | 0.00 | 0.00 |
| ATOM | 2007 | CA | VAL | 450 | 11.457 | 9.073 | −16.413 | 0.00 | 0.00 | 0.158 | 9.40 | 4.00 |
| ATOM | 2008 | HA | VAL | 450 | 12.108 | 9.204 | −15.548 | 0.00 | 0.00 | 0.053 | 0.00 | 0.00 |
| ATOM | 2009 | C | VAL | 450 | 12.266 | 8.438 | −17.501 | 0.00 | 0.00 | 0.396 | 9.82 | 4.00 |
| ATOM | 2010 | O | VAL | 450 | 13.414 | 8.058 | −17.291 | 0.00 | 0.00 | −0.396 | 8.17 | −17.40 |
| ATOM | 2011 | CB | VAL | 450 | 10.405 | 8.095 | −15.966 | 0.00 | 0.00 | −0.053 | 9.40 | 4.00 |
| ATOM | 2012 | HB | VAL | 450 | 9.874 | 8.499 | −15.104 | 0.00 | 0.00 | 0.053 | 0.00 | 0.00 |
| ATOM | 2013 | CG1 | VAL | 450 | 9.408 | 7.851 | −17.113 | 0.00 | 0.00 | −0.159 | 16.15 | 4.00 |
| ATOM | 2014 | HG11 | VAL | 450 | 8.646 | 7.142 | −16.788 | 0.00 | 0.00 | 0.053 | 0.00 | 0.00 |
| ATOM | 2015 | HG12 | VAL | 450 | 8.933 | 8.793 | −17.389 | 0.00 | 0.00 | 0.053 | 0.00 | 0.00 |
| ATOM | 2016 | HG13 | VAL | 450 | 9.937 | 7.445 | −17.975 | 0.00 | 0.00 | 0.053 | 0.00 | 0.00 |
| ATOM | 2017 | CG2 | VAL | 450 | 11.112 | 6.820 | −15.476 | 0.00 | 0.00 | −0.159 | 16.15 | 4.00 |
| ATOM | 2018 | HG21 | VAL | 450 | 10.368 | 6.095 | −15.146 | 0.00 | 0.00 | 0.053 | 0.00 | 0.00 |
| ATOM | 2019 | HG22 | VAL | 450 | 11.698 | 6.393 | −16.290 | 0.00 | 0.00 | 0.053 | 0.00 | 0.00 |
| ATOM | 2020 | HG23 | VAL | 450 | 11.772 | 7.067 | −14.644 | 0.00 | 0.00 | 0.053 | 0.00 | 0.00 |
| ATOM | 2021 | N | TYR | 451 | 11.687 | 8.325 | −18.708 | 0.00 | 0.00 | −0.650 | 9.00 | −17.40 |
| ATOM | 2022 | HN | TYR | 451 | 10.724 | 8.672 | −18.823 | 0.00 | 0.00 | 0.439 | 0.00 | 0.00 |
| ATOM | 2023 | CA | TYR | 451 | 12.342 | 7.746 | −19.843 | 0.00 | 0.00 | 0.158 | 9.40 | 4.00 |
| ATOM | 2024 | HA | TYR | 451 | 12.706 | 6.768 | −19.529 | 0.00 | 0.00 | 0.053 | 0.00 | 0.00 |
| ATOM | 2025 | C | TYR | 451 | 13.447 | 8.661 | −20.208 | 0.00 | 0.00 | 0.396 | 9.82 | 4.00 |
| ATOM | 2026 | O | TYR | 451 | 13.336 | 9.877 | −20.076 | 0.00 | 0.00 | −0.396 | 8.17 | −17.40 |
| ATOM | 2027 | CB | TYR | 451 | 11.425 | 7.570 | −21.069 | 0.00 | 0.00 | −0.106 | 12.77 | 4.00 |
| ATOM | 2028 | HB1 | TYR | 451 | 10.895 | 8.506 | −21.244 | 0.00 | 0.00 | 0.053 | 0.00 | 0.00 |
| ATOM | 2029 | HB2 | TYR | 451 | 10.717 | 6.767 | −20.862 | 0.00 | 0.00 | 0.053 | 0.00 | 0.00 |
| ATOM | 2030 | CG | TYR | 451 | 12.268 | 7.225 | −22.247 | 0.00 | 0.00 | 0.000 | 7.26 | 0.60 |
| ATOM | 2031 | CD1 | TYR | 451 | 12.802 | 5.966 | −22.390 | 0.00 | 0.00 | −0.127 | 10.80 | 0.60 |
| ATOM | 2032 | HD1 | TYR | 451 | 12.612 | 5.208 | −21.630 | 0.00 | 0.00 | 0.127 | 0.00 | 0.00 |
| ATOM | 2033 | CD2 | TYR | 451 | 12.507 | 8.167 | −23.220 | 0.00 | 0.00 | −0.127 | 10.80 | 0.60 |
| ATOM | 2034 | HD2 | TYR | 451 | 12.080 | 9.165 | −23.121 | 0.00 | 0.00 | 0.127 | 0.00 | 0.00 |
| ATOM | 2035 | CE1 | TYR | 451 | 13.574 | 5.656 | −23.484 | 0.00 | 0.00 | −0.127 | 10.80 | 0.60 |
| ATOM | 2036 | HE1 | TYR | 451 | 13.996 | 4.656 | −23.587 | 0.00 | 0.00 | 0.127 | 0.00 | 0.00 |
| ATOM | 2037 | CE2 | TYR | 451 | 13.278 | 7.863 | −24.316 | 0.00 | 0.00 | −0.127 | 10.80 | 0.60 |
| ATOM | 2038 | HE2 | TYR | 451 | 13.464 | 8.619 | −25.079 | 0.00 | 0.00 | 0.127 | 0.00 | 0.00 |
| ATOM | 2039 | CZ | TYR | 451 | 13.814 | 6.605 | −24.449 | 0.00 | 0.00 | 0.027 | 7.26 | 0.60 |
| ATOM | 2040 | OH | TYR | 451 | 14.607 | 6.287 | −25.573 | 0.00 | 0.00 | −0.451 | 10.94 | −17.40 |
| ATOM | 2041 | HH | TYR | 451 | 14.581 | 7.068 | −26.244 | 0.00 | 0.00 | 0.424 | 0.00 | 0.00 |
| ATOM | 2042 | N | GLY | 452 | 14.578 | 8.092 | −20.656 | 0.00 | 0.00 | −0.650 | 9.00 | −17.40 |
| ATOM | 2043 | HN | GLY | 452 | 14.628 | 7.075 | −20.809 | 0.00 | 0.00 | 0.439 | 0.00 | 0.00 |
| ATOM | 2044 | CA | GLY | 452 | 15.710 | 8.924 | −20.916 | 0.00 | 0.00 | 0.105 | 9.40 | 4.00 |
| ATOM | 2045 | HA1 | GLY | 452 | 15.282 | 9.860 | −21.274 | 0.00 | 0.00 | 0.053 | 0.00 | 0.00 |
| ATOM | 2046 | HA2 | GLY | 452 | 16.279 | 8.380 | −21.669 | 0.00 | 0.00 | 0.053 | 0.00 | 0.00 |
| ATOM | 2047 | C | GLY | 452 | 16.399 | 9.036 | −19.598 | 0.00 | 0.00 | 0.396 | 9.82 | 4.00 |
| ATOM | 2048 | O | GLY | 452 | 17.513 | 9.552 | −19.508 | 0.00 | 0.00 | −0.396 | 8.17 | −17.40 |
| ATOM | 2049 | N | LYS | 453 | 15.711 | 8.517 | −18.557 | 0.00 | 0.00 | −0.650 | 9.00 | −17.40 |
| ATOM | 2050 | HN | LYS | 453 | 14.787 | 8.117 | −18.776 | 0.00 | 0.00 | 0.439 | 0.00 | 0.00 |
| ATOM | 2051 | CA | LYS | 453 | 16.117 | 8.458 | −17.185 | 0.00 | 0.00 | 0.158 | 9.40 | 4.00 |
| ATOM | 2052 | HA | LYS | 453 | 15.237 | 8.422 | −16.542 | 0.00 | 0.00 | 0.053 | 0.00 | 0.00 |
| ATOM | 2053 | C | LYS | 453 | 16.921 | 9.663 | −16.847 | 0.00 | 0.00 | 0.396 | 9.82 | 4.00 |
| ATOM | 2054 | O | LYS | 453 | 17.949 | 9.569 | −16.178 | 0.00 | 0.00 | −0.396 | 8.17 | −17.40 |
| ATOM | 2055 | CB | LYS | 453 | 16.931 | 7.191 | −16.868 | 0.00 | 0.00 | −0.106 | 12.77 | 4.00 |
| ATOM | 2056 | HB1 | LYS | 453 | 17.248 | 7.150 | −15.826 | 0.00 | 0.00 | 0.053 | 0.00 | 0.00 |
| ATOM | 2057 | HB2 | LYS | 453 | 17.838 | 7.119 | −17.468 | 0.00 | 0.00 | 0.053 | 0.00 | 0.00 |
| ATOM | 2058 | CG | LYS | 453 | 16.138 | 5.906 | −17.129 | 0.00 | 0.00 | −0.106 | 12.77 | 4.00 |
| ATOM | 2059 | HG1 | LYS | 453 | 15.704 | 5.969 | −18.127 | 0.00 | 0.00 | 0.053 | 0.00 | 0.00 |
| ATOM | 2060 | HG2 | LYS | 453 | 15.356 | 5.828 | −16.374 | 0.00 | 0.00 | 0.053 | 0.00 | 0.00 |
| ATOM | 2061 | CD | LYS | 453 | 16.972 | 4.624 | −17.067 | 0.00 | 0.00 | −0.106 | 12.77 | 4.00 |
| ATOM | 2062 | HD1 | LYS | 453 | 18.038 | 4.794 | −17.221 | 0.00 | 0.00 | 0.053 | 0.00 | 0.00 |
| ATOM | 2063 | HD2 | LYS | 453 | 16.687 | 3.886 | −17.817 | 0.00 | 0.00 | 0.053 | 0.00 | 0.00 |
| ATOM | 2064 | CE | LYS | 453 | 16.868 | 3.897 | −15.725 | 0.00 | 0.00 | 0.099 | 12.77 | 4.00 |
| ATOM | 2065 | HE1 | LYS | 453 | 17.389 | 2.941 | −15.778 | 0.00 | 0.00 | 0.053 | 0.00 | 0.00 |
| ATOM | 2066 | HE2 | LYS | 453 | 15.821 | 3.717 | −15.480 | 0.00 | 0.00 | 0.053 | 0.00 | 0.00 |
| ATOM | 2067 | NZ | LYS | 453 | 17.478 | 4.719 | −14.657 | 0.00 | 0.00 | −0.045 | 13.25 | −39.20 |
| ATOM | 2068 | HZ1 | LYS | 453 | 17.404 | 4.223 | −13.757 | 0.00 | 0.00 | 0.280 | 0.00 | 0.00 |
| ATOM | 2069 | HZ2 | LYS | 453 | 18.470 | 4.886 | −14.876 | 0.00 | 0.00 | 0.280 | 0.00 | 0.00 |
| ATOM | 2070 | HZ3 | LYS | 453 | 16.985 | 5.621 | −14.593 | 0.00 | 0.00 | 0.280 | 0.00 | 0.00 |
| ATOM | 2071 | N | VAL | 454 | 16.470 | 10.840 | −17.321 | 0.00 | 0.00 | −0.650 | 9.00 | −17.40 |
| ATOM | 2072 | HN | VAL | 454 | 15.607 | 10.877 | −17.882 | 0.00 | 0.00 | 0.439 | 0.00 | 0.00 |
| ATOM | 2073 | CA | VAL | 454 | 17.197 | 12.038 | −17.041 | 0.00 | 0.00 | 0.158 | 9.40 | 4.00 |
| ATOM | 2074 | HA | VAL | 454 | 18.236 | 11.902 | −17.342 | 0.00 | 0.00 | 0.053 | 0.00 | 0.00 |
| ATOM | 2075 | C | VAL | 454 | 17.125 | 12.321 | −15.573 | 0.00 | 0.00 | 0.396 | 9.82 | 4.00 |
| ATOM | 2076 | O | VAL | 454 | 18.135 | 12.629 | −14.944 | 0.00 | 0.00 | −0.396 | 8.17 | −17.40 |
| ATOM | 2077 | CB | VAL | 454 | 16.668 | 13.227 | −17.784 | 0.00 | 0.00 | −0.053 | 9.40 | 4.00 |
| ATOM | 2078 | HB | VAL | 454 | 15.607 | 13.335 | −17.558 | 0.00 | 0.00 | 0.053 | 0.00 | 0.00 |
| ATOM | 2079 | CG1 | VAL | 454 | 17.440 | 14.475 | −17.327 | 0.00 | 0.00 | −0.159 | 16.15 | 4.00 |
| ATOM | 2080 | HG11 | VAL | 454 | 17.066 | 15.349 | −17.859 | 0.00 | 0.00 | 0.053 | 0.00 | 0.00 |
| ATOM | 2081 | HG12 | VAL | 454 | 17.302 | 14.616 | −16.255 | 0.00 | 0.00 | 0.053 | 0.00 | 0.00 |
| ATOM | 2082 | HG13 | VAL | 454 | 18.501 | 14.346 | −17.542 | 0.00 | 0.00 | 0.053 | 0.00 | 0.00 |
| ATOM | 2083 | CG2 | VAL | 454 | 16.798 | 12.939 | −19.290 | 0.00 | 0.00 | −0.159 | 16.15 | 4.00 |
| ATOM | 2084 | HG21 | VAL | 454 | 16.420 | 13.790 | −19.857 | 0.00 | 0.00 | 0.053 | 0.00 | 0.00 |
| ATOM | 2085 | HG22 | VAL | 454 | 17.846 | 12.774 | −19.540 | 0.00 | 0.00 | 0.053 | 0.00 | 0.00 |
| ATOM | 2086 | HG23 | VAL | 454 | 16.221 | 12.049 | −19.542 | 0.00 | 0.00 | 0.053 | 0.00 | 0.00 |
| ATOM | 2087 | N | ASP | 455 | 15.920 | 12.194 | −14.984 | 0.00 | 0.00 | −0.650 | 9.00 | −17.40 |
| ATOM | 2088 | HN | ASP | 455 | 15.114 | 11.880 | −15.544 | 0.00 | 0.00 | 0.439 | 0.00 | 0.00 |
| ATOM | 2089 | CA | ASP | 455 | 15.738 | 12.488 | −13.591 | 0.00 | 0.00 | 0.158 | 9.40 | 4.00 |
| ATOM | 2090 | HA | ASP | 455 | 16.437 | 13.285 | −13.338 | 0.00 | 0.00 | 0.053 | 0.00 | 0.00 |
| ATOM | 2091 | C | ASP | 455 | 16.033 | 11.232 | −12.834 | 0.00 | 0.00 | 0.396 | 9.82 | 4.00 |
| ATOM | 2092 | O | ASP | 455 | 16.031 | 10.136 | −13.394 | 0.00 | 0.00 | −0.396 | 8.17 | −17.40 |
| ATOM | 2093 | CB | ASP | 455 | 14.284 | 12.914 | −13.266 | 0.00 | 0.00 | −0.336 | 12.77 | 4.00 |
| ATOM | 2094 | HB1 | ASP | 455 | 13.654 | 12.025 | −13.282 | 0.00 | 0.00 | 0.053 | 0.00 | 0.00 |
| ATOM | 2095 | HB2 | ASP | 455 | 13.958 | 13.628 | −14.023 | 0.00 | 0.00 | 0.053 | 0.00 | 0.00 |
| ATOM | 2096 | CG | ASP | 455 | 14.118 | 13.579 | −11.894 | 0.00 | 0.00 | 0.297 | 9.82 | 4.00 |
| ATOM | 2097 | OD1 | ASP | 455 | 15.079 | 13.623 | −11.082 | 0.00 | 0.00 | −0.534 | 8.17 | −18.95 |
| ATOM | 2098 | OD2 | ASP | 455 | 12.983 | 14.070 | −11.647 | 0.00 | 0.00 | −0.534 | 8.17 | −18.95 |
| ATOM | 2099 | N | SER | 456 | 16.330 | 11.387 | −11.531 | 0.00 | 0.00 | −0.650 | 9.00 | −17.40 |
| ATOM | 2100 | HN | SER | 456 | 16.383 | 12.349 | −11.168 | 0.00 | 0.00 | 0.439 | 0.00 | 0.00 |
| ATOM | 2101 | CA | SER | 456 | 16.578 | 10.321 | −10.611 | 0.00 | 0.00 | 0.158 | 9.40 | 4.00 |
| ATOM | 2102 | HA | SER | 456 | 17.341 | 9.694 | −11.072 | 0.00 | 0.00 | 0.053 | 0.00 | 0.00 |
| ATOM | 2103 | C | SER | 456 | 15.278 | 9.609 | −10.440 | 0.00 | 0.00 | 0.396 | 9.82 | 4.00 |
| ATOM | 2104 | O | SER | 456 | 15.243 | 8.472 | −9.973 | 0.00 | 0.00 | −0.396 | 8.17 | −17.40 |
| ATOM | 2105 | CB | SER | 456 | 17.065 | 10.803 | −9.234 | 0.00 | 0.00 | 0.007 | 12.77 | 4.00 |
| ATOM | 2106 | HB1 | SER | 456 | 16.314 | 11.449 | −8.779 | 0.00 | 0.00 | 0.053 | 0.00 | 0.00 |
| ATOM | 2107 | HB2 | SER | 456 | 17.995 | 11.361 | −9.343 | 0.00 | 0.00 | 0.053 | 0.00 | 0.00 |
| ATOM | 2108 | OG | SER | 456 | 17.292 | 9.689 | −8.383 | 0.00 | 0.00 | −0.537 | 11.04 | −17.40 |
| ATOM | 2109 | HG | SER | 456 | 16.585 | 9.679 | −7.634 | 0.00 | 0.00 | 0.424 | 0.00 | 0.00 |
| ATOM | 2110 | N | PHE | 457 | 14.179 | 10.313 | −10.782 | 0.00 | 0.00 | −0.650 | 9.00 | −17.40 |
| ATOM | 2111 | HN | PHE | 457 | 14.331 | 11.282 | −11.095 | 0.00 | 0.00 | 0.439 | 0.00 | 0.00 |
| ATOM | 2112 | CA | PHE | 457 | 12.815 | 9.850 | −10.755 | 0.00 | 0.00 | 0.158 | 9.40 | 4.00 |
| ATOM | 2113 | HA | PHE | 457 | 12.492 | 9.901 | −9.715 | 0.00 | 0.00 | 0.053 | 0.00 | 0.00 |
| ATOM | 2114 | C | PHE | 457 | 12.833 | 8.444 | −11.278 | 0.00 | 0.00 | 0.396 | 9.82 | 4.00 |
| ATOM | 2115 | O | PHE | 457 | 12.847 | 8.223 | −12.487 | 0.00 | 0.00 | −0.396 | 8.17 | −17.40 |
| ATOM | 2116 | CB | PHE | 457 | 11.979 | 10.743 | −11.704 | 0.00 | 0.00 | −0.106 | 12.77 | 4.00 |
| ATOM | 2117 | HB1 | PHE | 457 | 12.281 | 10.517 | −12.727 | 0.00 | 0.00 | 0.053 | 0.00 | 0.00 |
| ATOM | 2118 | HB2 | PHE | 457 | 12.189 | 11.783 | −11.454 | 0.00 | 0.00 | 0.053 | 0.00 | 0.00 |
| ATOM | 2119 | CG | PHE | 457 | 10.496 | 10.570 | −11.639 | 0.00 | 0.00 | 0.000 | 7.26 | 0.60 |
| ATOM | 2120 | CD1 | PHE | 457 | 9.770 | 11.252 | −10.694 | 0.00 | 0.00 | −0.127 | 10.80 | 0.60 |
| ATOM | 2121 | HD1 | PHE | 457 | 10.289 | 11.904 | −9.992 | 0.00 | 0.00 | 0.127 | 0.00 | 0.00 |
| ATOM | 2122 | CD2 | PHE | 457 | 9.820 | 9.767 | −12.530 | 0.00 | 0.00 | −0.127 | 10.80 | 0.60 |
| ATOM | 2123 | HD2 | PHE | 457 | 10.374 | 9.224 | −13.296 | 0.00 | 0.00 | 0.127 | 0.00 | 0.00 |
| ATOM | 2124 | CE1 | PHE | 457 | 8.403 | 11.131 | −10.614 | 0.00 | 0.00 | −0.127 | 10.80 | 0.60 |
| ATOM | 2125 | HE1 | PHE | 457 | 7.850 | 11.676 | −9.849 | 0.00 | 0.00 | 0.127 | 0.00 | 0.00 |
| ATOM | 2126 | CE2 | PHE | 457 | 8.451 | 9.644 | −12.461 | 0.00 | 0.00 | −0.127 | 10.80 | 0.60 |
| ATOM | 2127 | HE2 | PHE | 457 | 7.929 | 9.004 | −13.173 | 0.00 | 0.00 | 0.127 | 0.00 | 0.00 |
| ATOM | 2128 | CZ | PHE | 457 | 7.737 | 10.322 | −11.501 | 0.00 | 0.00 | −0.127 | 10.80 | 0.60 |
| ATOM | 2129 | HZ | PHE | 457 | 6.653 | 10.218 | −11.445 | 0.00 | 0.00 | 0.127 | 0.00 | 0.00 |
| ATOM | 2130 | N | TRP | 458 | 12.878 | 7.445 | −10.367 | 0.00 | 0.00 | −0.650 | 9.00 | −17.40 |
| ATOM | 2131 | HN | TRP | 458 | 12.835 | 7.654 | −9.359 | 0.00 | 0.00 | 0.439 | 0.00 | 0.00 |
| ATOM | 2132 | CA | TRP | 458 | 12.987 | 6.089 | −10.826 | 0.00 | 0.00 | 0.158 | 9.40 | 4.00 |
| ATOM | 2133 | HA | TRP | 458 | 13.736 | 6.015 | −11.615 | 0.00 | 0.00 | 0.053 | 0.00 | 0.00 |
| ATOM | 2134 | C | TRP | 458 | 11.680 | 5.618 | −11.366 | 0.00 | 0.00 | 0.396 | 9.82 | 4.00 |
| ATOM | 2135 | O | TRP | 458 | 11.644 | 4.800 | −12.282 | 0.00 | 0.00 | −0.396 | 8.17 | −17.40 |
| ATOM | 2136 | CB | TRP | 458 | 13.507 | 5.054 | −9.800 | 0.00 | 0.00 | −0.106 | 12.77 | 4.00 |
| ATOM | 2137 | HB1 | TRP | 458 | 14.382 | 5.484 | −9.313 | 0.00 | 0.00 | 0.053 | 0.00 | 0.00 |
| ATOM | 2138 | HB2 | TRP | 458 | 13.764 | 4.148 | −10.348 | 0.00 | 0.00 | 0.053 | 0.00 | 0.00 |
| ATOM | 2139 | CG | TRP | 458 | 12.561 | 4.641 | −8.702 | 0.00 | 0.00 | 0.000 | 7.26 | 0.60 |
| ATOM | 2140 | CD1 | TRP | 458 | 11.461 | 5.272 | −8.214 | 0.00 | 0.00 | −0.177 | 10.80 | 0.60 |
| ATOM | 2141 | HD1 | TRP | 458 | 11.095 | 6.241 | −8.553 | 0.00 | 0.00 | 0.127 | 0.00 | 0.00 |
| ATOM | 2142 | CD2 | TRP | 458 | 12.671 | 3.400 | −7.987 | 0.00 | 0.00 | 0.000 | 6.80 | 0.60 |
| ATOM | 2143 | NE1 | TRP | 458 | 10.878 | 4.510 | −7.230 | 0.00 | 0.00 | −0.292 | 9.00 | −17.40 |
| ATOM | 2144 | HE1 | TRP | 458 | 10.036 | 4.762 | −6.693 | 0.00 | 0.00 | 0.393 | 0.00 | 0.00 |
| ATOM | 2145 | CE2 | TRP | 458 | 11.611 | 3.351 | −7.083 | 0.00 | 0.00 | −0.050 | 6.80 | 0.60 |
| ATOM | 2146 | CE3 | TRP | 458 | 13.575 | 2.380 | −8.080 | 0.00 | 0.00 | −0.127 | 10.80 | 0.60 |
| ATOM | 2147 | HE3 | TRP | 458 | 14.400 | 2.417 | −8.791 | 0.00 | 0.00 | 0.127 | 0.00 | 0.00 |
| ATOM | 2148 | CZ2 | TRP | 458 | 11.438 | 2.278 | −6.256 | 0.00 | 0.00 | −0.127 | 10.80 | 0.60 |
| ATOM | 2149 | HZ2 | TRP | 458 | 10.607 | 2.234 | −5.552 | 0.00 | 0.00 | 0.127 | 0.00 | 0.00 |
| ATOM | 2150 | CZ3 | TRP | 458 | 13.402 | 1.302 | −7.239 | 0.00 | 0.00 | −0.127 | 10.80 | 0.60 |
| ATOM | 2151 | HZ3 | TRP | 458 | 14.107 | 0.472 | −7.282 | 0.00 | 0.00 | 0.127 | 0.00 | 0.00 |
| ATOM | 2152 | CH2 | TRP | 458 | 12.354 | 1.253 | −6.345 | 0.00 | 0.00 | −0.127 | 10.80 | 0.60 |
| ATOM | 2153 | HH2 | TRP | 458 | 12.248 | 0.385 | −5.695 | 0.00 | 0.00 | 0.127 | 0.00 | 0.00 |
| ATOM | 2154 | N | LYS | 459 | 10.555 | 6.088 | −10.799 | 0.00 | 0.00 | −0.650 | 9.00 | −17.40 |
| ATOM | 2155 | HN | LYS | 459 | 10.585 | 6.764 | −10.023 | 0.00 | 0.00 | 0.439 | 0.00 | 0.00 |
| ATOM | 2156 | CA | LYS | 459 | 9.317 | 5.606 | −11.323 | 0.00 | 0.00 | 0.158 | 9.40 | 4.00 |
| ATOM | 2157 | HA | LYS | 459 | 9.488 | 5.246 | −12.338 | 0.00 | 0.00 | 0.053 | 0.00 | 0.00 |
| ATOM | 2158 | C | LYS | 459 | 8.342 | 6.722 | −11.327 | 0.00 | 0.00 | 0.396 | 9.82 | 4.00 |
| ATOM | 2159 | O | LYS | 459 | 8.469 | 7.678 | −10.564 | 0.00 | 0.00 | −0.396 | 8.17 | −17.40 |
| ATOM | 2160 | CB | LYS | 459 | 8.711 | 4.431 | −10.524 | 0.00 | 0.00 | −0.106 | 12.77 | 4.00 |
| ATOM | 2161 | HB1 | LYS | 459 | 9.440 | 3.620 | −10.516 | 0.00 | 0.00 | 0.053 | 0.00 | 0.00 |
| ATOM | 2162 | HB2 | LYS | 459 | 7.792 | 4.125 | −11.023 | 0.00 | 0.00 | 0.053 | 0.00 | 0.00 |
| ATOM | 2163 | CG | LYS | 459 | 8.352 | 4.728 | −9.062 | 0.00 | 0.00 | −0.106 | 12.77 | 4.00 |
| ATOM | 2164 | HG1 | LYS | 459 | 9.205 | 5.221 | −8.596 | 0.00 | 0.00 | 0.053 | 0.00 | 0.00 |
| ATOM | 2165 | HG2 | LYS | 459 | 8.135 | 3.782 | −8.566 | 0.00 | 0.00 | 0.053 | 0.00 | 0.00 |
| ATOM | 2166 | CD | LYS | 459 | 7.135 | 5.637 | −8.873 | 0.00 | 0.00 | −0.106 | 12.77 | 4.00 |
| ATOM | 2167 | HD1 | LYS | 459 | 6.272 | 5.343 | −9.470 | 0.00 | 0.00 | 0.053 | 0.00 | 0.00 |
| ATOM | 2168 | HD2 | LYS | 459 | 7.320 | 6.677 | −9.140 | 0.00 | 0.00 | 0.053 | 0.00 | 0.00 |
| ATOM | 2169 | CE | LYS | 459 | 6.621 | 5.684 | −7.432 | 0.00 | 0.00 | 0.099 | 12.77 | 4.00 |
| ATOM | 2170 | HE1 | LYS | 459 | 6.306 | 4.691 | −7.111 | 0.00 | 0.00 | 0.053 | 0.00 | 0.00 |
| ATOM | 2171 | HE2 | LYS | 459 | 5.770 | 6.361 | −7.357 | 0.00 | 0.00 | 0.053 | 0.00 | 0.00 |
| ATOM | 2172 | NZ | LYS | 459 | 7.687 | 6.160 | −6.523 | 0.00 | 0.00 | −0.045 | 13.25 | −39.20 |
| ATOM | 2173 | HZ1 | LYS | 459 | 7.329 | 6.187 | −5.558 | 0.00 | 0.00 | 0.280 | 0.00 | 0.00 |
| ATOM | 2174 | HZ2 | LYS | 459 | 8.492 | 5.520 | −6.572 | 0.00 | 0.00 | 0.280 | 0.00 | 0.00 |
| ATOM | 2175 | HZ3 | LYS | 459 | 7.983 | 7.105 | −6.806 | 0.00 | 0.00 | 0.280 | 0.00 | 0.00 |
| ATOM | 2176 | N | ASN | 460 | 7.345 | 6.625 | −12.225 | 0.00 | 0.00 | −0.650 | 9.00 | −17.40 |
| ATOM | 2177 | HN | ASN | 460 | 7.323 | 5.832 | −12.882 | 0.00 | 0.00 | 0.439 | 0.00 | 0.00 |
| ATOM | 2178 | CA | ASN | 460 | 6.319 | 7.614 | −12.273 | 0.00 | 0.00 | 0.158 | 9.40 | 4.00 |
| ATOM | 2179 | HA | ASN | 460 | 6.724 | 8.462 | −11.721 | 0.00 | 0.00 | 0.053 | 0.00 | 0.00 |
| ATOM | 2180 | C | ASN | 460 | 5.148 | 6.978 | −11.616 | 0.00 | 0.00 | 0.396 | 9.82 | 4.00 |
| ATOM | 2181 | O | ASN | 460 | 4.683 | 5.917 | −12.025 | 0.00 | 0.00 | −0.396 | 8.17 | −17.40 |
| ATOM | 2182 | CB | ASN | 460 | 5.907 | 8.027 | −13.694 | 0.00 | 0.00 | −0.106 | 12.77 | 4.00 |
| ATOM | 2183 | HB1 | ASN | 460 | 5.312 | 7.210 | −14.102 | 0.00 | 0.00 | 0.053 | 0.00 | 0.00 |
| ATOM | 2184 | HB2 | ASN | 460 | 6.827 | 8.181 | −14.258 | 0.00 | 0.00 | 0.053 | 0.00 | 0.00 |
| ATOM | 2185 | CG | ASN | 460 | 5.098 | 9.309 | −13.565 | 0.00 | 0.00 | 0.396 | 9.82 | 4.00 |
| ATOM | 2186 | OD1 | ASN | 460 | 5.629 | 10.409 | −13.713 | 0.00 | 0.00 | −0.396 | 8.17 | −17.40 |
| ATOM | 2187 | ND2 | ASN | 460 | 3.777 | 9.169 | −13.276 | 0.00 | 0.00 | −0.879 | 13.25 | −17.40 |
| ATOM | 2188 | HD21 | ASN | 460 | 3.372 | 8.229 | −13.160 | 0.00 | 0.00 | 0.439 | 0.00 | 0.00 |
| ATOM | 2189 | HD22 | ASN | 460 | 3.181 | 10.003 | −13.173 | 0.00 | 0.00 | 0.439 | 0.00 | 0.00 |
| ATOM | 2190 | N | LYS | 461 | 4.651 | 7.644 | −10.563 | 0.00 | 0.00 | −0.650 | 9.00 | −17.40 |
| ATOM | 2191 | HN | LYS | 461 | 5.034 | 8.576 | −10.350 | 0.00 | 0.00 | 0.439 | 0.00 | 0.00 |
| ATOM | 2192 | CA | LYS | 461 | 3.621 | 7.130 | −9.725 | 0.00 | 0.00 | 0.158 | 9.40 | 4.00 |
| ATOM | 2193 | HA | LYS | 461 | 3.531 | 6.052 | −9.859 | 0.00 | 0.00 | 0.053 | 0.00 | 0.00 |
| ATOM | 2194 | C | LYS | 461 | 2.323 | 7.763 | −10.063 | 0.00 | 0.00 | 0.396 | 9.82 | 4.00 |
| ATOM | 2195 | O | LYS | 461 | 2.232 | 8.972 | −10.263 | 0.00 | 0.00 | −0.396 | 8.17 | −17.40 |
| ATOM | 2196 | CB | LYS | 461 | 3.862 | 7.501 | −8.263 | 0.00 | 0.00 | −0.106 | 12.77 | 4.00 |
| ATOM | 2197 | HB1 | LYS | 461 | 4.124 | 8.554 | −8.162 | 0.00 | 0.00 | 0.053 | 0.00 | 0.00 |
| ATOM | 2198 | HB2 | LYS | 461 | 4.676 | 6.914 | −7.837 | 0.00 | 0.00 | 0.053 | 0.00 | 0.00 |
| ATOM | 2199 | CG | LYS | 461 | 2.626 | 7.252 | −7.424 | 0.00 | 0.00 | −0.106 | 12.77 | 4.00 |
| ATOM | 2200 | HG1 | LYS | 461 | 1.770 | 7.785 | −7.838 | 0.00 | 0.00 | 0.053 | 0.00 | 0.00 |
| ATOM | 2201 | HG2 | LYS | 461 | 2.783 | 7.595 | −6.401 | 0.00 | 0.00 | 0.053 | 0.00 | 0.00 |
| ATOM | 2202 | CD | LYS | 461 | 2.299 | 5.775 | −7.388 | 0.00 | 0.00 | −0.106 | 12.77 | 4.00 |
| ATOM | 2203 | HD1 | LYS | 461 | 3.205 | 5.236 | −7.110 | 0.00 | 0.00 | 0.053 | 0.00 | 0.00 |
| ATOM | 2204 | HD2 | LYS | 461 | 1.960 | 5.481 | −8.381 | 0.00 | 0.00 | 0.053 | 0.00 | 0.00 |
| ATOM | 2205 | CE | LYS | 461 | 1.210 | 5.368 | −6.400 | 0.00 | 0.00 | 0.099 | 12.77 | 4.00 |
| ATOM | 2206 | HE1 | LYS | 461 | 0.953 | 6.175 | −5.713 | 0.00 | 0.00 | 0.053 | 0.00 | 0.00 |
| ATOM | 2207 | HE2 | LYS | 461 | 1.507 | 4.519 | −5.784 | 0.00 | 0.00 | 0.053 | 0.00 | 0.00 |
| ATOM | 2208 | NZ | LYS | 461 | −0.036 | 4.980 | −7.074 | 0.00 | 0.00 | −0.045 | 13.25 | −39.20 |
| ATOM | 2209 | HZ1 | LYS | 461 | −0.739 | 4.714 | −6.370 | 0.00 | 0.00 | 0.280 | 0.00 | 0.00 |
| ATOM | 2210 | HZ2 | LYS | 461 | −0.388 | 5.773 | −7.628 | 0.00 | 0.00 | 0.280 | 0.00 | 0.00 |
| ATOM | 2211 | HZ3 | LYS | 461 | 0.145 | 4.180 | −7.697 | 0.00 | 0.00 | 0.280 | 0.00 | 0.00 |
| ATOM | 2212 | N | LYS | 462 | 1.287 | 6.912 | −10.136 | 0.00 | 0.00 | −0.650 | 9.00 | −17.40 |
| ATOM | 2213 | HN | LYS | 462 | 1.489 | 5.910 | −10.008 | 0.00 | 0.00 | 0.439 | 0.00 | 0.00 |
| ATOM | 2214 | CA | LYS | 462 | −0.070 | 7.281 | −10.376 | 0.00 | 0.00 | 0.158 | 9.40 | 4.00 |
| ATOM | 2215 | HA | LYS | 462 | −0.145 | 7.895 | −11.274 | 0.00 | 0.00 | 0.053 | 0.00 | 0.00 |
| ATOM | 2216 | C | LYS | 462 | −0.610 | 8.061 | −9.224 | 0.00 | 0.00 | 0.396 | 9.82 | 4.00 |
| ATOM | 2217 | O | LYS | 462 | −1.193 | 9.127 | −9.411 | 0.00 | 0.00 | −0.396 | 8.17 | −17.40 |
| ATOM | 2218 | CB | LYS | 462 | −0.927 | 6.009 | −10.553 | 0.00 | 0.00 | −0.106 | 12.77 | 4.00 |
| ATOM | 2219 | HB1 | LYS | 462 | −0.576 | 5.273 | −9.830 | 0.00 | 0.00 | 0.053 | 0.00 | 0.00 |
| ATOM | 2220 | HB2 | LYS | 462 | −0.787 | 5.658 | −11.575 | 0.00 | 0.00 | 0.053 | 0.00 | 0.00 |
| ATOM | 2221 | CG | LYS | 462 | −2.438 | 6.147 | −10.340 | 0.00 | 0.00 | −0.106 | 12.77 | 4.00 |
| ATOM | 2222 | HG1 | LYS | 462 | −2.919 | 5.263 | −10.758 | 0.00 | 0.00 | 0.053 | 0.00 | 0.00 |
| ATOM | 2223 | HG2 | LYS | 462 | −2.770 | 7.049 | −10.854 | 0.00 | 0.00 | 0.053 | 0.00 | 0.00 |
| ATOM | 2224 | CD | LYS | 462 | −2.892 | 6.264 | −8.875 | 0.00 | 0.00 | −0.106 | 12.77 | 4.00 |
| ATOM | 2225 | HD1 | LYS | 462 | −3.806 | 6.857 | −8.847 | 0.00 | 0.00 | 0.053 | 0.00 | 0.00 |
| ATOM | 2226 | HD2 | LYS | 462 | −2.099 | 6.753 | −8.309 | 0.00 | 0.00 | 0.053 | 0.00 | 0.00 |
| ATOM | 2227 | CE | LYS | 462 | −3.187 | 4.926 | −8.197 | 0.00 | 0.00 | 0.099 | 12.77 | 4.00 |
| ATOM | 2228 | HE1 | LYS | 462 | −2.302 | 4.291 | −8.230 | 0.00 | 0.00 | 0.053 | 0.00 | 0.00 |
| ATOM | 2229 | HE2 | LYS | 462 | −4.006 | 4.423 | −8.711 | 0.00 | 0.00 | 0.053 | 0.00 | 0.00 |
| ATOM | 2230 | NZ | LYS | 462 | −3.569 | 5.151 | −6.783 | 0.00 | 0.00 | −0.045 | 13.25 | −39.20 |
| ATOM | 2231 | HZ1 | LYS | 462 | −3.766 | 4.246 | −6.332 | 0.00 | 0.00 | 0.280 | 0.00 | 0.00 |
| ATOM | 2232 | HZ2 | LYS | 462 | −2.796 | 5.619 | −6.288 | 0.00 | 0.00 | 0.280 | 0.00 | 0.00 |
| ATOM | 2233 | HZ3 | LYS | 462 | −4.410 | 5.744 | −6.744 | 0.00 | 0.00 | 0.280 | 0.00 | 0.00 |
| ATOM | 2234 | N | ILE | 463 | −0.412 | 7.573 | −7.983 | 0.00 | 0.00 | −0.650 | 9.00 | −17.40 |
| ATOM | 2235 | HN | ILE | 463 | 0.209 | 6.772 | −7.799 | 0.00 | 0.00 | 0.439 | 0.00 | 0.00 |
| ATOM | 2236 | CA | ILE | 463 | −1.124 | 8.244 | −6.938 | 0.00 | 0.00 | 0.158 | 9.40 | 4.00 |
| ATOM | 2237 | HA | ILE | 463 | −1.930 | 8.857 | −7.342 | 0.00 | 0.00 | 0.053 | 0.00 | 0.00 |
| ATOM | 2238 | C | ILE | 463 | −0.225 | 9.133 | −6.164 | 0.00 | 0.00 | 0.396 | 9.82 | 4.00 |
| ATOM | 2239 | O | ILE | 463 | 0.955 | 8.856 | −5.962 | 0.00 | 0.00 | −0.396 | 8.17 | −17.40 |
| ATOM | 2240 | CB | ILE | 463 | −1.833 | 7.363 | −5.940 | 0.00 | 0.00 | −0.053 | 9.40 | 4.00 |
| ATOM | 2241 | HB | ILE | 463 | −2.279 | 6.521 | −6.469 | 0.00 | 0.00 | 0.053 | 0.00 | 0.00 |
| ATOM | 2242 | CG1 | ILE | 463 | −2.934 | 8.153 | −5.220 | 0.00 | 0.00 | −0.106 | 12.77 | 4.00 |
| ATOM | 2243 | HG11 | ILE | 463 | −3.591 | 8.702 | −5.895 | 0.00 | 0.00 | 0.053 | 0.00 | 0.00 |
| ATOM | 2244 | HG12 | ILE | 463 | −2.552 | 8.902 | −4.526 | 0.00 | 0.00 | 0.053 | 0.00 | 0.00 |
| ATOM | 2245 | CG2 | ILE | 463 | −0.817 | 6.862 | −4.916 | 0.00 | 0.00 | −0.159 | 16.15 | 4.00 |
| ATOM | 2246 | HG21 | ILE | 463 | −1.318 | 6.223 | −4.189 | 0.00 | 0.00 | 0.053 | 0.00 | 0.00 |
| ATOM | 2247 | HG22 | ILE | 463 | −0.039 | 6.292 | −5.424 | 0.00 | 0.00 | 0.053 | 0.00 | 0.00 |
| ATOM | 2248 | HG23 | ILE | 463 | −0.367 | 7.712 | −4.403 | 0.00 | 0.00 | 0.053 | 0.00 | 0.00 |
| ATOM | 2249 | CD1 | ILE | 463 | −3.869 | 7.279 | −4.383 | 0.00 | 0.00 | −0.159 | 16.15 | 4.00 |
| ATOM | 2250 | HD11 | ILE | 463 | −4.621 | 7.906 | −3.904 | 0.00 | 0.00 | 0.053 | 0.00 | 0.00 |
| ATOM | 2251 | HD12 | ILE | 463 | −4.361 | 6.551 | −5.028 | 0.00 | 0.00 | 0.053 | 0.00 | 0.00 |
| ATOM | 2252 | HD13 | ILE | 463 | −3.292 | 6.756 | −3.620 | 0.00 | 0.00 | 0.053 | 0.00 | 0.00 |
| ATOM | 2253 | N | TYR | 464 | −0.799 | 10.259 | −5.710 | 0.00 | 0.00 | −0.650 | 9.00 | −17.40 |
| ATOM | 2254 | HN | TYR | 464 | −1.780 | 10.460 | −5.950 | 0.00 | 0.00 | 0.439 | 0.00 | 0.00 |
| ATOM | 2255 | CA | TYR | 464 | −0.071 | 11.182 | −4.900 | 0.00 | 0.00 | 0.158 | 9.40 | 4.00 |
| ATOM | 2256 | HA | TYR | 464 | 0.905 | 11.398 | −5.335 | 0.00 | 0.00 | 0.053 | 0.00 | 0.00 |
| ATOM | 2257 | C | TYR | 464 | 0.053 | 10.483 | −3.597 | 0.00 | 0.00 | 0.396 | 9.82 | 4.00 |
| ATOM | 2258 | O | TYR | 464 | −0.493 | 9.389 | −3.455 | 0.00 | 0.00 | −0.396 | 8.17 | −17.40 |
| ATOM | 2259 | CB | TYR | 464 | −0.826 | 12.514 | −4.672 | 0.00 | 0.00 | −0.106 | 12.77 | 4.00 |
| ATOM | 2260 | HB1 | TYR | 464 | −1.726 | 12.347 | −4.080 | 0.00 | 0.00 | 0.053 | 0.00 | 0.00 |
| ATOM | 2261 | HB2 | TYR | 464 | −1.118 | 12.956 | −5.625 | 0.00 | 0.00 | 0.053 | 0.00 | 0.00 |
| ATOM | 2262 | CG | TYR | 464 | 0.043 | 13.493 | −3.944 | 0.00 | 0.00 | 0.000 | 7.26 | 0.60 |
| ATOM | 2263 | CD1 | TYR | 464 | 1.055 | 14.149 | −4.604 | 0.00 | 0.00 | −0.127 | 10.80 | 0.60 |
| ATOM | 2264 | HD1 | TYR | 464 | 1.214 | 13.944 | −5.663 | 0.00 | 0.00 | 0.127 | 0.00 | 0.00 |
| ATOM | 2265 | CD2 | TYR | 464 | −0.148 | 13.778 | −2.612 | 0.00 | 0.00 | −0.127 | 10.80 | 0.60 |
| ATOM | 2266 | HD2 | TYR | 464 | −0.949 | 13.285 | −2.061 | 0.00 | 0.00 | 0.127 | 0.00 | 0.00 |
| ATOM | 2267 | CE1 | TYR | 464 | 1.871 | 15.052 | −3.974 | 0.00 | 0.00 | −0.127 | 10.80 | 0.60 |
| ATOM | 2268 | HE1 | TYR | 464 | 2.667 | 15.552 | −4.526 | 0.00 | 0.00 | 0.127 | 0.00 | 0.00 |
| ATOM | 2269 | CE2 | TYR | 464 | 0.667 | 14.683 | −1.971 | 0.00 | 0.00 | −0.127 | 10.80 | 0.60 |
| ATOM | 2270 | HE2 | TYR | 464 | 0.507 | 14.896 | −0.914 | 0.00 | 0.00 | 0.127 | 0.00 | 0.00 |
| ATOM | 2271 | CZ | TYR | 464 | 1.678 | 15.321 | −2.645 | 0.00 | 0.00 | 0.027 | 7.26 | 0.60 |
| ATOM | 2272 | OH | TYR | 464 | 2.511 | 16.247 | −1.983 | 0.00 | 0.00 | −0.451 | 10.94 | −17.40 |
| ATOM | 2273 | HH | TYR | 464 | 3.351 | 16.422 | −2.552 | 0.00 | 0.00 | 0.424 | 0.00 | 0.00 |
| ATOM | 2274 | N | LEU | 465 | 0.769 | 11.113 | −2.638 | 0.00 | 0.00 | −0.650 | 9.00 | −17.40 |
| ATOM | 2275 | HN | LEU | 465 | 1.183 | 12.018 | −2.903 | 0.00 | 0.00 | 0.439 | 0.00 | 0.00 |
| ATOM | 2276 | CA | LEU | 465 | 1.022 | 10.671 | −1.294 | 0.00 | 0.00 | 0.158 | 9.40 | 4.00 |
| ATOM | 2277 | HA | LEU | 465 | 1.886 | 10.009 | −1.346 | 0.00 | 0.00 | 0.053 | 0.00 | 0.00 |
| ATOM | 2278 | C | LEU | 465 | −0.210 | 9.962 | −0.846 | 0.00 | 0.00 | 0.396 | 9.82 | 4.00 |
| ATOM | 2279 | O | LEU | 465 | −1.310 | 10.349 | −1.242 | 0.00 | 0.00 | −0.396 | 8.17 | −17.40 |
| ATOM | 2280 | CB | LEU | 465 | 1.337 | 11.880 | −0.379 | 0.00 | 0.00 | −0.106 | 12.77 | 4.00 |
| ATOM | 2281 | HB1 | LEU | 465 | 0.422 | 12.467 | −0.298 | 0.00 | 0.00 | 0.053 | 0.00 | 0.00 |
| ATOM | 2282 | HB2 | LEU | 465 | 2.138 | 12.443 | −0.858 | 0.00 | 0.00 | 0.053 | 0.00 | 0.00 |
| ATOM | 2283 | CG | LEU | 465 | 1.807 | 11.626 | 1.074 | 0.00 | 0.00 | −0.053 | 9.40 | 4.00 |
| ATOM | 2284 | HG | LEU | 465 | 2.729 | 11.045 | 1.068 | 0.00 | 0.00 | 0.053 | 0.00 | 0.00 |
| ATOM | 2285 | CD1 | LEU | 465 | 2.062 | 12.967 | 1.782 | 0.00 | 0.00 | −0.159 | 16.15 | 4.00 |
| ATOM | 2286 | HD11 | LEU | 465 | 2.392 | 12.782 | 2.804 | 0.00 | 0.00 | 0.053 | 0.00 | 0.00 |
| ATOM | 2287 | HD12 | LEU | 465 | 2.833 | 13.521 | 1.246 | 0.00 | 0.00 | 0.053 | 0.00 | 0.00 |
| ATOM | 2288 | HD13 | LEU | 465 | 1.142 | 13.551 | 1.798 | 0.00 | 0.00 | 0.053 | 0.00 | 0.00 |
| ATOM | 2289 | CD2 | LEU | 465 | 0.838 | 10.762 | 1.893 | 0.00 | 0.00 | −0.159 | 16.15 | 4.00 |
| ATOM | 2290 | HD21 | LEU | 465 | 1.233 | 10.627 | 2.900 | 0.00 | 0.00 | 0.053 | 0.00 | 0.00 |
| ATOM | 2291 | HD22 | LEU | 465 | −0.132 | 11.255 | 1.947 | 0.00 | 0.00 | 0.053 | 0.00 | 0.00 |
| ATOM | 2292 | HD23 | LEU | 465 | 0.724 | 9.789 | 1.414 | 0.00 | 0.00 | 0.053 | 0.00 | 0.00 |
| ATOM | 2293 | N | ASP | 466 | −0.036 | 8.907 | −0.027 | 0.00 | 0.00 | −0.650 | 9.00 | −17.40 |
| ATOM | 2294 | HN | ASP | 466 | 0.913 | 8.726 | 0.330 | 0.00 | 0.00 | 0.439 | 0.00 | 0.00 |
| ATOM | 2295 | CA | ASP | 466 | −1.086 | 8.021 | 0.379 | 0.00 | 0.00 | 0.158 | 9.40 | 4.00 |
| ATOM | 2296 | HA | ASP | 466 | −1.352 | 7.427 | −0.496 | 0.00 | 0.00 | 0.053 | 0.00 | 0.00 |
| ATOM | 2297 | C | ASP | 466 | −2.211 | 8.873 | 0.837 | 0.00 | 0.00 | 0.396 | 9.82 | 4.00 |
| ATOM | 2298 | O | ASP | 466 | −3.375 | 8.565 | 0.588 | 0.00 | 0.00 | −0.396 | 8.17 | −17.40 |
| ATOM | 2299 | CB | ASP | 466 | −0.695 | 7.139 | 1.576 | 0.00 | 0.00 | −0.336 | 12.77 | 4.00 |
| ATOM | 2300 | HB1 | ASP | 466 | −1.570 | 6.573 | 1.896 | 0.00 | 0.00 | 0.053 | 0.00 | 0.00 |
| ATOM | 2301 | HB2 | ASP | 466 | −0.346 | 7.780 | 2.386 | 0.00 | 0.00 | 0.053 | 0.00 | 0.00 |
| ATOM | 2302 | CG | ASP | 466 | 0.413 | 6.191 | 1.145 | 0.00 | 0.00 | 0.297 | 9.82 | 4.00 |
| ATOM | 2303 | OD1 | ASP | 466 | 0.720 | 6.150 | −0.076 | 0.00 | 0.00 | −0.534 | 8.17 | −18.95 |
| ATOM | 2304 | OD2 | ASP | 466 | 0.970 | 5.499 | 2.038 | 0.00 | 0.00 | −0.534 | 8.17 | −18.95 |
| ATOM | 2305 | N | ILE | 467 | −1.891 | 9.990 | 1.510 | 0.00 | 0.00 | −0.650 | 9.00 | −17.40 |
| ATOM | 2306 | HN | ILE | 467 | −0.919 | 10.205 | 1.775 | 0.00 | 0.00 | 0.439 | 0.00 | 0.00 |
| ATOM | 2307 | CA | ILE | 467 | −2.969 | 10.862 | 1.838 | 0.00 | 0.00 | 0.158 | 9.40 | 4.00 |
| ATOM | 2308 | HA | ILE | 467 | −3.828 | 10.223 | 2.045 | 0.00 | 0.00 | 0.053 | 0.00 | 0.00 |
| ATOM | 2309 | C | ILE | 467 | −3.183 | 11.740 | 0.636 | 0.00 | 0.00 | 0.396 | 9.82 | 4.00 |
| ATOM | 2310 | O | ILE | 467 | −2.868 | 12.929 | 0.612 | 0.00 | 0.00 | −0.396 | 8.17 | −17.40 |
| ATOM | 2311 | CB | ILE | 467 | −2.746 | 11.636 | 3.120 | 0.00 | 0.00 | −0.053 | 9.40 | 4.00 |
| ATOM | 2312 | HB | ILE | 467 | −2.568 | 10.945 | 3.944 | 0.00 | 0.00 | 0.053 | 0.00 | 0.00 |
| ATOM | 2313 | CG1 | ILE | 467 | −4.044 | 12.308 | 3.607 | 0.00 | 0.00 | −0.106 | 12.77 | 4.00 |
| ATOM | 2314 | HG11 | ILE | 467 | −3.831 | 12.811 | 4.550 | 0.00 | 0.00 | 0.053 | 0.00 | 0.00 |
| ATOM | 2315 | HG12 | ILE | 467 | −4.796 | 11.531 | 3.743 | 0.00 | 0.00 | 0.053 | 0.00 | 0.00 |
| ATOM | 2316 | CG2 | ILE | 467 | −1.529 | 12.558 | 2.968 | 0.00 | 0.00 | −0.159 | 16.15 | 4.00 |
| ATOM | 2317 | HG21 | ILE | 467 | −1.374 | 13.112 | 3.894 | 0.00 | 0.00 | 0.053 | 0.00 | 0.00 |
| ATOM | 2318 | HG22 | ILE | 467 | −0.644 | 11.960 | 2.751 | 0.00 | 0.00 | 0.053 | 0.00 | 0.00 |
| ATOM | 2319 | HG23 | ILE | 467 | −1.703 | 13.258 | 2.151 | 0.00 | 0.00 | 0.053 | 0.00 | 0.00 |
| ATOM | 2320 | CD1 | ILE | 467 | −4.634 | 13.351 | 2.659 | 0.00 | 0.00 | −0.159 | 16.15 | 4.00 |
| ATOM | 2321 | HD11 | ILE | 467 | −5.544 | 13.764 | 3.093 | 0.00 | 0.00 | 0.053 | 0.00 | 0.00 |
| ATOM | 2322 | HD12 | ILE | 467 | −3.911 | 14.152 | 2.503 | 0.00 | 0.00 | 0.053 | 0.00 | 0.00 |
| ATOM | 2323 | HD13 | ILE | 467 | −4.868 | 12.883 | 1.703 | 0.00 | 0.00 | 0.053 | 0.00 | 0.00 |
| ATOM | 2324 | N | ILE | 468 | −3.710 | 11.108 | −0.430 | 0.00 | 0.00 | −0.650 | 9.00 | −17.40 |
| ATOM | 2325 | HN | ILE | 468 | −3.869 | 10.094 | −0.339 | 0.00 | 0.00 | 0.439 | 0.00 | 0.00 |
| ATOM | 2326 | CA | ILE | 468 | −4.071 | 11.711 | −1.678 | 0.00 | 0.00 | 0.158 | 9.40 | 4.00 |
| ATOM | 2327 | HA | ILE | 468 | −3.185 | 12.269 | −1.981 | 0.00 | 0.00 | 0.053 | 0.00 | 0.00 |
| ATOM | 2328 | C | ILE | 468 | −5.240 | 12.570 | −1.356 | 0.00 | 0.00 | 0.396 | 9.82 | 4.00 |
| ATOM | 2329 | O | ILE | 468 | −5.531 | 13.541 | −2.053 | 0.00 | 0.00 | −0.396 | 8.17 | −17.40 |
| ATOM | 2330 | CB | ILE | 468 | −4.474 | 10.667 | −2.688 | 0.00 | 0.00 | −0.053 | 9.40 | 4.00 |
| ATOM | 2331 | HB | ILE | 468 | −3.694 | 9.906 | −2.697 | 0.00 | 0.00 | 0.053 | 0.00 | 0.00 |
| ATOM | 2332 | CG1 | ILE | 468 | −4.470 | 11.199 | −4.135 | 0.00 | 0.00 | −0.106 | 12.77 | 4.00 |
| ATOM | 2333 | HG11 | ILE | 468 | −4.625 | 10.354 | −4.806 | 0.00 | 0.00 | 0.053 | 0.00 | 0.00 |
| ATOM | 2334 | HG12 | ILE | 468 | −3.504 | 11.669 | −4.319 | 0.00 | 0.00 | 0.053 | 0.00 | 0.00 |
| ATOM | 2335 | CG2 | ILE | 468 | −5.824 | 10.086 | −2.233 | 0.00 | 0.00 | −0.159 | 16.15 | 4.00 |
| ATOM | 2336 | HG21 | ILE | 468 | −6.150 | 9.324 | −2.941 | 0.00 | 0.00 | 0.053 | 0.00 | 0.00 |
| ATOM | 2337 | HG22 | ILE | 468 | −5.713 | 9.639 | −1.245 | 0.00 | 0.00 | 0.053 | 0.00 | 0.00 |
| ATOM | 2338 | HG23 | ILE | 468 | −6.567 | 10.882 | −2.190 | 0.00 | 0.00 | 0.053 | 0.00 | 0.00 |
| ATOM | 2339 | CD1 | ILE | 468 | −5.545 | 12.234 | −4.454 | 0.00 | 0.00 | −0.159 | 16.15 | 4.00 |
| ATOM | 2340 | HD11 | ILE | 468 | −5.455 | 12.543 | −5.495 | 0.00 | 0.00 | 0.053 | 0.00 | 0.00 |
| ATOM | 2341 | HD12 | ILE | 468 | −6.530 | 11.798 | −4.288 | 0.00 | 0.00 | 0.053 | 0.00 | 0.00 |
| ATOM | 2342 | HD13 | ILE | 468 | −5.419 | 13.101 | −3.806 | 0.00 | 0.00 | 0.053 | 0.00 | 0.00 |
| ATOM | 2343 | N | HIS | 469 | −5.930 | 12.185 | −0.264 | 0.00 | 0.00 | −0.650 | 9.00 | −17.40 |
| ATOM | 2344 | HN | HIS | 469 | −5.569 | 11.367 | 0.247 | 0.00 | 0.00 | 0.439 | 0.00 | 0.00 |
| ATOM | 2345 | CA | HIS | 469 | −7.115 | 12.799 | 0.258 | 0.00 | 0.00 | 0.158 | 9.40 | 4.00 |
| ATOM | 2346 | HA | HIS | 469 | −7.982 | 12.446 | −0.300 | 0.00 | 0.00 | 0.053 | 0.00 | 0.00 |
| ATOM | 2347 | C | HIS | 469 | −7.006 | 14.281 | 0.124 | 0.00 | 0.00 | 0.396 | 9.82 | 4.00 |
| ATOM | 2348 | O | HIS | 469 | −5.915 | 14.850 | 0.085 | 0.00 | 0.00 | −0.396 | 8.17 | −17.40 |
| ATOM | 2349 | CB | HIS | 469 | −7.318 | 12.524 | 1.758 | 0.00 | 0.00 | −0.106 | 12.77 | 4.00 |
| ATOM | 2350 | HB1 | HIS | 469 | −8.287 | 12.927 | 2.052 | 0.00 | 0.00 | 0.053 | 0.00 | 0.00 |
| ATOM | 2351 | HB2 | HIS | 469 | −6.515 | 13.015 | 2.308 | 0.00 | 0.00 | 0.053 | 0.00 | 0.00 |
| ATOM | 2352 | CG | HIS | 469 | −7.299 | 11.070 | 2.126 | 0.00 | 0.00 | 0.241 | 7.26 | 0.60 |
| ATOM | 2353 | ND1 | HIS | 469 | −6.237 | 10.232 | 1.865 | 0.00 | 0.00 | −0.481 | 9.25 | −17.40 |
| ATOM | 2354 | CD2 | HIS | 469 | −8.215 | 10.313 | 2.789 | 0.00 | 0.00 | −0.177 | 10.80 | 0.60 |
| ATOM | 2355 | HD2 | HIS | 469 | −9.181 | 10.674 | 3.142 | 0.00 | 0.00 | 0.127 | 0.00 | 0.00 |
| ATOM | 2356 | CE1 | HIS | 469 | −6.559 | 9.019 | 2.381 | 0.00 | 0.00 | 0.064 | 10.80 | 0.60 |
| ATOM | 2357 | HE1 | HIS | 469 | −5.910 | 8.145 | 2.329 | 0.00 | 0.00 | 0.127 | 0.00 | 0.00 |
| ATOM | 2358 | NE2 | HIS | 469 | −7.749 | 9.020 | 2.953 | 0.00 | 0.00 | −0.292 | 9.25 | −17.40 |
| ATOM | 2359 | HE2 | HIS | 469 | −8.225 | 8.234 | 3.418 | 0.00 | 0.00 | 0.393 | 0.00 | 0.00 |
| ATOM | 2360 | N | THR | 470 | −8.172 | 14.940 | 0.029 | 0.00 | 0.00 | −0.650 | 9.00 | −17.40 |
| ATOM | 2361 | HN | THR | 470 | −9.051 | 14.404 | 0.051 | 0.00 | 0.00 | 0.439 | 0.00 | 0.00 |
| ATOM | 2362 | CA | THR | 470 | −8.230 | 16.363 | −0.102 | 0.00 | 0.00 | 0.158 | 9.40 | 4.00 |
| ATOM | 2363 | HA | THR | 470 | −7.632 | 16.648 | −0.968 | 0.00 | 0.00 | 0.053 | 0.00 | 0.00 |
| ATOM | 2364 | C | THR | 470 | −7.678 | 16.965 | 1.143 | 0.00 | 0.00 | 0.396 | 9.82 | 4.00 |
| ATOM | 2365 | O | THR | 470 | −7.049 | 18.021 | 1.109 | 0.00 | 0.00 | −0.396 | 8.17 | −17.40 |
| ATOM | 2366 | CB | THR | 470 | −9.629 | 16.885 | −0.241 | 0.00 | 0.00 | 0.060 | 9.40 | 4.00 |
| ATOM | 2367 | HB | THR | 470 | −10.096 | 16.411 | −1.104 | 0.00 | 0.00 | 0.053 | 0.00 | 0.00 |
| ATOM | 2368 | OG1 | THR | 470 | −9.598 | 18.291 | −0.439 | 0.00 | 0.00 | −0.537 | 11.04 | −17.40 |
| ATOM | 2369 | HG1 | THR | 470 | −8.629 | 18.629 | −0.350 | 0.00 | 0.00 | 0.424 | 0.00 | 0.00 |
| ATOM | 2370 | CG2 | THR | 470 | −10.413 | 16.547 | 1.038 | 0.00 | 0.00 | −0.159 | 16.15 | 4.00 |
| ATOM | 2371 | HG21 | THR | 470 | −11.433 | 16.922 | 0.949 | 0.00 | 0.00 | 0.053 | 0.00 | 0.00 |
| ATOM | 2372 | HG22 | THR | 470 | −10.434 | 15.466 | 1.177 | 0.00 | 0.00 | 0.053 | 0.00 | 0.00 |
| ATOM | 2373 | HG23 | THR | 470 | −9.928 | 17.013 | 1.896 | 0.00 | 0.00 | 0.053 | 0.00 | 0.00 |
| ATOM | 2374 | N | TYR | 471 | −7.884 | 16.277 | 2.279 | 0.00 | 0.00 | −0.650 | 9.00 | −17.40 |
| ATOM | 2375 | HN | TYR | 471 | −8.318 | 15.345 | 2.222 | 0.00 | 0.00 | 0.439 | 0.00 | 0.00 |
| ATOM | 2376 | CA | TYR | 471 | −7.523 | 16.791 | 3.565 | 0.00 | 0.00 | 0.158 | 9.40 | 4.00 |
| ATOM | 2377 | HA | TYR | 471 | −8.144 | 17.652 | 3.813 | 0.00 | 0.00 | 0.053 | 0.00 | 0.00 |
| ATOM | 2378 | C | TYR | 471 | −6.090 | 17.215 | 3.565 | 0.00 | 0.00 | 0.396 | 9.82 | 4.00 |
| ATOM | 2379 | O | TYR | 471 | −5.765 | 18.276 | 4.095 | 0.00 | 0.00 | −0.396 | 8.17 | −17.40 |
| ATOM | 2380 | CB | TYR | 471 | −7.678 | 15.742 | 4.681 | 0.00 | 0.00 | −0.106 | 12.77 | 4.00 |
| ATOM | 2381 | HB1 | TYR | 471 | −7.354 | 16.196 | 5.618 | 0.00 | 0.00 | 0.053 | 0.00 | 0.00 |
| ATOM | 2382 | HB2 | TYR | 471 | −7.053 | 14.886 | 4.428 | 0.00 | 0.00 | 0.053 | 0.00 | 0.00 |
| ATOM | 2383 | CG | TYR | 471 | −9.114 | 15.350 | 4.749 | 0.00 | 0.00 | 0.000 | 7.26 | 0.60 |
| ATOM | 2384 | CD1 | TYR | 471 | −10.019 | 16.104 | 5.460 | 0.00 | 0.00 | −0.127 | 10.80 | 0.60 |
| ATOM | 2385 | HD1 | TYR | 471 | −9.684 | 17.001 | 5.980 | 0.00 | 0.00 | 0.127 | 0.00 | 0.00 |
| ATOM | 2386 | CD2 | TYR | 471 | −9.555 | 14.221 | 4.098 | 0.00 | 0.00 | −0.127 | 10.80 | 0.60 |
| ATOM | 2387 | HD2 | TYR | 471 | −8.848 | 13.616 | 3.531 | 0.00 | 0.00 | 0.127 | 0.00 | 0.00 |
| ATOM | 2388 | CE1 | TYR | 471 | −11.343 | 15.736 | 5.520 | 0.00 | 0.00 | −0.127 | 10.80 | 0.60 |
| ATOM | 2389 | HE1 | TYR | 471 | −12.051 | 16.341 | 6.087 | 0.00 | 0.00 | 0.127 | 0.00 | 0.00 |
| ATOM | 2390 | CE2 | TYR | 471 | −10.877 | 13.847 | 4.154 | 0.00 | 0.00 | −0.127 | 10.80 | 0.60 |
| ATOM | 2391 | HE2 | TYR | 471 | −11.213 | 12.950 | 3.634 | 0.00 | 0.00 | 0.127 | 0.00 | 0.00 |
| ATOM | 2392 | CZ | TYR | 471 | −11.774 | 14.606 | 4.866 | 0.00 | 0.00 | 0.027 | 7.26 | 0.60 |
| ATOM | 2393 | OH | TYR | 471 | −13.131 | 14.226 | 4.926 | 0.00 | 0.00 | −0.451 | 10.94 | −17.40 |
| ATOM | 2394 | HH | TYR | 471 | −13.723 | 15.068 | 4.946 | 0.00 | 0.00 | 0.424 | 0.00 | 0.00 |
| ATOM | 2395 | N | MET | 472 | −5.184 | 16.417 | 2.966 | 0.00 | 0.00 | −0.650 | 9.00 | −17.40 |
| ATOM | 2396 | HN | MET | 472 | −5.474 | 15.566 | 2.464 | 0.00 | 0.00 | 0.439 | 0.00 | 0.00 |
| ATOM | 2397 | CA | MET | 472 | −3.803 | 16.792 | 3.051 | 0.00 | 0.00 | 0.158 | 9.40 | 4.00 |
| ATOM | 2398 | HA | MET | 472 | −3.517 | 16.956 | 4.090 | 0.00 | 0.00 | 0.053 | 0.00 | 0.00 |
| ATOM | 2399 | C | MET | 472 | −3.558 | 18.052 | 2.287 | 0.00 | 0.00 | 0.396 | 9.82 | 4.00 |
| ATOM | 2400 | O | MET | 472 | −4.471 | 18.816 | 1.981 | 0.00 | 0.00 | −0.396 | 8.17 | −17.40 |
| ATOM | 2401 | CB | MET | 472 | −2.793 | 15.787 | 2.483 | 0.00 | 0.00 | −0.106 | 12.77 | 4.00 |
| ATOM | 2402 | HB1 | MET | 472 | −2.950 | 14.781 | 2.873 | 0.00 | 0.00 | 0.053 | 0.00 | 0.00 |
| ATOM | 2403 | HB2 | MET | 472 | −1.765 | 16.058 | 2.722 | 0.00 | 0.00 | 0.053 | 0.00 | 0.00 |
| ATOM | 2404 | CG | MET | 472 | −2.876 | 15.684 | 0.963 | 0.00 | 0.00 | −0.041 | 12.77 | 4.00 |
| ATOM | 2405 | HG1 | MET | 472 | −3.497 | 16.501 | 0.596 | 0.00 | 0.00 | 0.053 | 0.00 | 0.00 |
| ATOM | 2406 | HG2 | MET | 472 | −3.320 | 14.722 | 0.707 | 0.00 | 0.00 | 0.053 | 0.00 | 0.00 |
| ATOM | 2407 | SD | MET | 472 | −1.266 | 15.792 | 0.130 | 0.00 | 0.00 | −0.130 | 16.39 | −6.40 |
| ATOM | 2408 | CE | MET | 472 | −0.503 | 14.302 | 0.832 | 0.00 | 0.00 | −0.094 | 16.15 | 4.00 |
| ATOM | 2409 | HE1 | MET | 472 | 0.507 | 14.190 | 0.437 | 0.00 | 0.00 | 0.053 | 0.00 | 0.00 |
| ATOM | 2410 | HE2 | MET | 472 | −0.461 | 14.392 | 1.917 | 0.00 | 0.00 | 0.053 | 0.00 | 0.00 |
| ATOM | 2411 | HE3 | MET | 472 | −1.097 | 13.428 | 0.563 | 0.00 | 0.00 | 0.053 | 0.00 | 0.00 |
| ATOM | 2412 | N | GLU | 473 | −2.273 | 18.264 | 1.942 | 0.00 | 0.00 | −0.650 | 9.00 | −17.40 |
| ATOM | 2413 | HN | GLU | 473 | −1.584 | 17.536 | 2.177 | 0.00 | 0.00 | 0.439 | 0.00 | 0.00 |
| ATOM | 2414 | CA | GLU | 473 | −1.806 | 19.438 | 1.269 | 0.00 | 0.00 | 0.158 | 9.40 | 4.00 |
| ATOM | 2415 | HA | GLU | 473 | −1.975 | 20.299 | 1.915 | 0.00 | 0.00 | 0.053 | 0.00 | 0.00 |
| ATOM | 2416 | C | GLU | 473 | −2.564 | 19.589 | −0.002 | 0.00 | 0.00 | 0.396 | 9.82 | 4.00 |
| ATOM | 2417 | O | GLU | 473 | −3.012 | 20.686 | −0.329 | 0.00 | 0.00 | −0.396 | 8.17 | −17.40 |
| ATOM | 2418 | CB | GLU | 473 | −0.315 | 19.344 | 0.902 | 0.00 | 0.00 | −0.106 | 12.77 | 4.00 |
| ATOM | 2419 | HB1 | GLU | 473 | −0.076 | 18.512 | 0.240 | 0.00 | 0.00 | 0.053 | 0.00 | 0.00 |
| ATOM | 2420 | HB2 | GLU | 473 | 0.343 | 19.214 | 1.761 | 0.00 | 0.00 | 0.053 | 0.00 | 0.00 |
| ATOM | 2421 | CG | GLU | 473 | 0.223 | 20.581 | 0.183 | 0.00 | 0.00 | −0.336 | 12.77 | 4.00 |
| ATOM | 2422 | HG1 | GLU | 473 | 0.089 | 21.436 | 0.846 | 0.00 | 0.00 | 0.053 | 0.00 | 0.00 |
| ATOM | 2423 | HG2 | GLU | 473 | −0.347 | 20.705 | −0.737 | 0.00 | 0.00 | 0.053 | 0.00 | 0.00 |
| ATOM | 2424 | CD | GLU | 473 | 1.696 | 20.342 | −0.113 | 0.00 | 0.00 | 0.297 | 9.82 | 4.00 |
| ATOM | 2425 | OE1 | GLU | 473 | 2.014 | 19.291 | −0.731 | 0.00 | 0.00 | −0.534 | 8.17 | −18.95 |
| ATOM | 2426 | OE2 | GLU | 473 | 2.524 | 21.206 | 0.282 | 0.00 | 0.00 | −0.534 | 8.17 | −18.95 |
| ATOM | 2427 | N | VAL | 474 | −2.730 | 18.482 | −0.752 | 0.00 | 0.00 | −0.650 | 9.00 | −17.40 |
| ATOM | 2428 | HN | VAL | 474 | −2.331 | 17.583 | −0.445 | 0.00 | 0.00 | 0.439 | 0.00 | 0.00 |
| ATOM | 2429 | CA | VAL | 474 | −3.465 | 18.563 | −1.979 | 0.00 | 0.00 | 0.158 | 9.40 | 4.00 |
| ATOM | 2430 | HA | VAL | 474 | −2.910 | 19.246 | −2.622 | 0.00 | 0.00 | 0.053 | 0.00 | 0.00 |
| ATOM | 2431 | C | VAL | 474 | −4.812 | 19.084 | −1.623 | 0.00 | 0.00 | 0.396 | 9.82 | 4.00 |
| ATOM | 2432 | O | VAL | 474 | −5.289 | 18.874 | −0.510 | 0.00 | 0.00 | −0.396 | 8.17 | −17.40 |
| ATOM | 2433 | CB | VAL | 474 | −3.631 | 17.240 | −2.661 | 0.00 | 0.00 | −0.053 | 9.40 | 4.00 |
| ATOM | 2434 | HB | VAL | 474 | −4.289 | 17.376 | −3.519 | 0.00 | 0.00 | 0.053 | 0.00 | 0.00 |
| ATOM | 2435 | CG1 | VAL | 474 | −2.245 | 16.752 | −3.115 | 0.00 | 0.00 | −0.159 | 16.15 | 4.00 |
| ATOM | 2436 | HG11 | VAL | 474 | −2.344 | 15.788 | −3.615 | 0.00 | 0.00 | 0.053 | 0.00 | 0.00 |
| ATOM | 2437 | HG12 | VAL | 474 | −1.813 | 17.476 | −3.806 | 0.00 | 0.00 | 0.053 | 0.00 | 0.00 |
| ATOM | 2438 | HG13 | VAL | 474 | −1.595 | 16.645 | −2.247 | 0.00 | 0.00 | 0.053 | 0.00 | 0.00 |
| ATOM | 2439 | CG2 | VAL | 474 | −4.329 | 16.278 | −1.684 | 0.00 | 0.00 | −0.159 | 16.15 | 4.00 |
| ATOM | 2440 | HG21 | VAL | 474 | −4.460 | 15.307 | −2.161 | 0.00 | 0.00 | 0.053 | 0.00 | 0.00 |
| ATOM | 2441 | HG22 | VAL | 474 | −3.719 | 16.163 | −0.788 | 0.00 | 0.00 | 0.053 | 0.00 | 0.00 |
| ATOM | 2442 | HG23 | VAL | 474 | −5.303 | 16.682 | −1.409 | 0.00 | 0.00 | 0.053 | 0.00 | 0.00 |
| ATOM | 2443 | N | HIS | 475 | −5.456 | 19.814 | −2.548 | 0.00 | 0.00 | −0.650 | 9.00 | −17.40 |
| ATOM | 2444 | HN | HIS | 475 | −5.045 | 19.974 | −3.479 | 0.00 | 0.00 | 0.439 | 0.00 | 0.00 |
| ATOM | 2445 | CA | HIS | 475 | −6.729 | 20.361 | −2.200 | 0.00 | 0.00 | 0.158 | 9.40 | 4.00 |
| ATOM | 2446 | HA | HIS | 475 | −6.996 | 20.059 | −1.187 | 0.00 | 0.00 | 0.053 | 0.00 | 0.00 |
| ATOM | 2447 | C | HIS | 475 | −7.769 | 19.864 | −3.150 | 0.00 | 0.00 | 0.396 | 9.82 | 4.00 |
| ATOM | 2448 | O | HIS | 475 | −7.496 | 19.607 | −4.322 | 0.00 | 0.00 | −0.396 | 8.17 | −17.40 |
| ATOM | 2449 | CB | HIS | 475 | −6.761 | 21.898 | −2.188 | 0.00 | 0.00 | −0.106 | 12.77 | 4.00 |
| ATOM | 2450 | HB1 | HIS | 475 | −7.764 | 22.313 | −2.287 | 0.00 | 0.00 | 0.053 | 0.00 | 0.00 |
| ATOM | 2451 | HB2 | HIS | 475 | −6.184 | 22.351 | −2.994 | 0.00 | 0.00 | 0.053 | 0.00 | 0.00 |
| ATOM | 2452 | CG | HIS | 475 | −6.211 | 22.499 | −0.926 | 0.00 | 0.00 | 0.241 | 7.26 | 0.60 |
| ATOM | 2453 | ND1 | HIS | 475 | −6.913 | 22.551 | 0.258 | 0.00 | 0.00 | −0.481 | 9.25 | −17.40 |
| ATOM | 2454 | CD2 | HIS | 475 | −5.013 | 23.092 | −0.672 | 0.00 | 0.00 | −0.177 | 10.80 | 0.60 |
| ATOM | 2455 | HD2 | HIS | 475 | −4.214 | 23.215 | −1.403 | 0.00 | 0.00 | 0.127 | 0.00 | 0.00 |
| ATOM | 2456 | CE1 | HIS | 475 | −6.112 | 23.169 | 1.163 | 0.00 | 0.00 | 0.064 | 10.80 | 0.60 |
| ATOM | 2457 | HE1 | HIS | 475 | −6.396 | 23.356 | 2.199 | 0.00 | 0.00 | 0.127 | 0.00 | 0.00 |
| ATOM | 2458 | NE2 | HIS | 475 | −4.949 | 23.518 | 0.643 | 0.00 | 0.00 | −0.292 | 9.25 | −17.40 |
| ATOM | 2459 | HE2 | HIS | 475 | −4.169 | 24.000 | 1.112 | 0.00 | 0.00 | 0.393 | 0.00 | 0.00 |
| ATOM | 2460 | N | ALA | 476 | −9.006 | 19.713 | −2.627 | 0.00 | 0.00 | −0.650 | 9.00 | −17.40 |
| ATOM | 2461 | HN | ALA | 476 | −9.151 | 19.959 | −1.637 | 0.00 | 0.00 | 0.439 | 0.00 | 0.00 |
| ATOM | 2462 | CA | ALA | 476 | −10.127 | 19.228 | −3.380 | 0.00 | 0.00 | 0.158 | 9.40 | 4.00 |
| ATOM | 2463 | HA | ALA | 476 | −9.804 | 18.336 | −3.917 | 0.00 | 0.00 | 0.053 | 0.00 | 0.00 |
| ATOM | 2464 | C | ALA | 476 | −10.538 | 20.305 | −4.322 | 0.00 | 0.00 | 0.396 | 9.82 | 4.00 |
| ATOM | 2465 | O | ALA | 476 | −9.715 | 20.835 | −5.063 | 0.00 | 0.00 | −0.396 | 8.17 | −17.40 |
| ATOM | 2466 | CB | ALA | 476 | −11.353 | 18.894 | −2.513 | 0.00 | 0.00 | −0.159 | 16.15 | 4.00 |
| ATOM | 2467 | HB1 | ALA | 476 | −12.161 | 18.533 | −3.149 | 0.00 | 0.00 | 0.053 | 0.00 | 0.00 |
| ATOM | 2468 | HB2 | ALA | 476 | −11.088 | 18.122 | −1.790 | 0.00 | 0.00 | 0.053 | 0.00 | 0.00 |
| ATOM | 2469 | HB3 | ALA | 476 | −11.679 | 19.789 | −1.984 | 0.00 | 0.00 | 0.053 | 0.00 | 0.00 |
| ATOM | 2470 | N | THR | 477 | −11.844 | 20.636 | −4.341 | 0.00 | 0.00 | −0.650 | 9.00 | −17.40 |
| ATOM | 2471 | HN | THR | 477 | −12.516 | 20.151 | −3.729 | 0.00 | 0.00 | 0.439 | 0.00 | 0.00 |
| ATOM | 2472 | CA | THR | 477 | −12.281 | 21.673 | −5.226 | 0.00 | 0.00 | 0.158 | 9.40 | 4.00 |
| ATOM | 2473 | HA | THR | 477 | −11.442 | 21.965 | −5.858 | 0.00 | 0.00 | 0.053 | 0.00 | 0.00 |
| ATOM | 2474 | C | THR | 477 | −12.744 | 22.833 | −4.406 | 0.00 | 0.00 | 0.396 | 9.82 | 4.00 |
| ATOM | 2475 | O | THR | 477 | −13.531 | 22.686 | −3.471 | 0.00 | 0.00 | −0.396 | 8.17 | −17.40 |
| ATOM | 2476 | CB | THR | 477 | −13.410 | 21.268 | −6.134 | 0.00 | 0.00 | 0.060 | 9.40 | 4.00 |
| ATOM | 2477 | HB | THR | 477 | −13.132 | 20.368 | −6.683 | 0.00 | 0.00 | 0.053 | 0.00 | 0.00 |
| ATOM | 2478 | OG1 | THR | 477 | −13.666 | 22.294 | −7.082 | 0.00 | 0.00 | −0.537 | 11.04 | −17.40 |
| ATOM | 2479 | HG1 | THR | 477 | −13.027 | 23.084 | −6.915 | 0.00 | 0.00 | 0.424 | 0.00 | 0.00 |
| ATOM | 2480 | CG2 | THR | 477 | −14.669 | 20.986 | −5.297 | 0.00 | 0.00 | −0.159 | 16.15 | 4.00 |
| ATOM | 2481 | HG21 | THR | 477 | −15.486 | 20.693 | −5.956 | 0.00 | 0.00 | 0.053 | 0.00 | 0.00 |
| ATOM | 2482 | HG22 | THR | 477 | −14.464 | 20.180 | −4.592 | 0.00 | 0.00 | 0.053 | 0.00 | 0.00 |
| ATOM | 2483 | HG23 | THR | 477 | −14.950 | 21.885 | −4.749 | 0.00 | 0.00 | 0.053 | 0.00 | 0.00 |
| ATOM | 2484 | N | VAL | 478 | −12.216 | 24.026 | −4.729 | 0.00 | 0.00 | −0.650 | 9.00 | −17.40 |
| ATOM | 2485 | HN | VAL | 478 | −11.500 | 24.071 | −5.468 | 0.00 | 0.00 | 0.439 | 0.00 | 0.00 |
| ATOM | 2486 | CA | VAL | 478 | −12.617 | 25.236 | −4.079 | 0.00 | 0.00 | 0.158 | 9.40 | 4.00 |
| ATOM | 2487 | HA | VAL | 478 | −13.696 | 25.287 | −4.224 | 0.00 | 0.00 | 0.053 | 0.00 | 0.00 |
| ATOM | 2488 | C | VAL | 478 | −11.867 | 26.314 | −4.783 | 0.00 | 0.00 | 0.396 | 9.82 | 4.00 |
| ATOM | 2489 | O | VAL | 478 | −11.321 | 26.092 | −5.862 | 0.00 | 0.00 | −0.396 | 8.17 | −17.40 |
| ATOM | 2490 | CB | VAL | 478 | −12.260 | 25.315 | −2.619 | 0.00 | 0.00 | −0.053 | 9.40 | 4.00 |
| ATOM | 2491 | HB | VAL | 478 | −12.525 | 24.374 | −2.137 | 0.00 | 0.00 | 0.053 | 0.00 | 0.00 |
| ATOM | 2492 | CG1 | VAL | 478 | −10.750 | 25.564 | −2.486 | 0.00 | 0.00 | −0.159 | 16.15 | 4.00 |
| ATOM | 2493 | HG11 | VAL | 478 | −10.482 | 25.623 | −1.431 | 0.00 | 0.00 | 0.053 | 0.00 | 0.00 |
| ATOM | 2494 | HG12 | VAL | 478 | −10.204 | 24.745 | −2.954 | 0.00 | 0.00 | 0.053 | 0.00 | 0.00 |
| ATOM | 2495 | HG13 | VAL | 478 | −10.491 | 26.501 | −2.979 | 0.00 | 0.00 | 0.053 | 0.00 | 0.00 |
| ATOM | 2496 | CG2 | VAL | 478 | −13.127 | 26.397 | −1.956 | 0.00 | 0.00 | −0.159 | 16.15 | 4.00 |
| ATOM | 2497 | HG21 | VAL | 478 | −12.878 | 26.464 | −0.897 | 0.00 | 0.00 | 0.053 | 0.00 | 0.00 |
| ATOM | 2498 | HG22 | VAL | 478 | −12.939 | 27.358 | −2.435 | 0.00 | 0.00 | 0.053 | 0.00 | 0.00 |
| ATOM | 2499 | HG23 | VAL | 478 | −14.180 | 26.137 | −2.065 | 0.00 | 0.00 | 0.053 | 0.00 | 0.00 |
| ATOM | 2500 | N | TYR | 479 | −11.830 | 27.523 | −4.201 | 0.00 | 0.00 | −0.650 | 9.00 | −17.40 |
| ATOM | 2501 | HN | TYR | 479 | −12.328 | 27.699 | −3.317 | 0.00 | 0.00 | 0.439 | 0.00 | 0.00 |
| ATOM | 2502 | CA | TYR | 479 | −11.083 | 28.557 | −4.838 | 0.00 | 0.00 | 0.158 | 9.40 | 4.00 |
| ATOM | 2503 | HA | TYR | 479 | −11.281 | 28.458 | −5.905 | 0.00 | 0.00 | 0.053 | 0.00 | 0.00 |
| ATOM | 2504 | C | TYR | 479 | −9.648 | 28.305 | −4.491 | 0.00 | 0.00 | 0.396 | 9.82 | 4.00 |
| ATOM | 2505 | O | TYR | 479 | −9.160 | 28.773 | −3.463 | 0.00 | 0.00 | −0.396 | 8.17 | −17.40 |
| ATOM | 2506 | CB | TYR | 479 | −11.470 | 29.956 | −4.324 | 0.00 | 0.00 | −0.106 | 12.77 | 4.00 |
| ATOM | 2507 | HB1 | TYR | 479 | −11.218 | 30.027 | −3.266 | 0.00 | 0.00 | 0.053 | 0.00 | 0.00 |
| ATOM | 2508 | HB2 | TYR | 479 | −12.542 | 30.100 | −4.462 | 0.00 | 0.00 | 0.053 | 0.00 | 0.00 |
| ATOM | 2509 | CG | TYR | 479 | −10.715 | 30.981 | −5.096 | 0.00 | 0.00 | 0.000 | 7.26 | 0.60 |
| ATOM | 2510 | CD1 | TYR | 479 | −11.145 | 31.356 | −6.347 | 0.00 | 0.00 | −0.127 | 10.80 | 0.60 |
| ATOM | 2511 | HD1 | TYR | 479 | −12.038 | 30.896 | −6.770 | 0.00 | 0.00 | 0.127 | 0.00 | 0.00 |
| ATOM | 2512 | CD2 | TYR | 479 | −9.590 | 31.575 | −4.571 | 0.00 | 0.00 | −0.127 | 10.80 | 0.60 |
| ATOM | 2513 | HD2 | TYR | 479 | −9.242 | 31.291 | −3.578 | 0.00 | 0.00 | 0.127 | 0.00 | 0.00 |
| ATOM | 2514 | CE1 | TYR | 479 | −10.462 | 32.304 | −7.070 | 0.00 | 0.00 | −0.127 | 10.80 | 0.60 |
| ATOM | 2515 | HE1 | TYR | 479 | −10.812 | 32.591 | −8.061 | 0.00 | 0.00 | 0.127 | 0.00 | 0.00 |
| ATOM | 2516 | CE2 | TYR | 479 | −8.902 | 32.524 | −5.290 | 0.00 | 0.00 | −0.127 | 10.80 | 0.60 |
| ATOM | 2517 | HE2 | TYR | 479 | −8.011 | 32.987 | −4.867 | 0.00 | 0.00 | 0.127 | 0.00 | 0.00 |
| ATOM | 2518 | CZ | TYR | 479 | −9.337 | 32.889 | −6.541 | 0.00 | 0.00 | 0.027 | 7.26 | 0.60 |
| ATOM | 2519 | OH | TYR | 479 | −8.634 | 33.863 | −7.281 | 0.00 | 0.00 | −0.451 | 10.94 | −17.40 |
| ATOM | 2520 | HH | TYR | 479 | −7.792 | 34.149 | −6.761 | 0.00 | 0.00 | 0.424 | 0.00 | 0.00 |
| ATOM | 2521 | N | GLY | 480 | −8.934 | 27.541 | −5.346 | 0.00 | 0.00 | −0.650 | 9.00 | −17.40 |
| ATOM | 2522 | HN | GLY | 480 | −9.388 | 27.146 | −6.182 | 0.00 | 0.00 | 0.439 | 0.00 | 0.00 |
| ATOM | 2523 | CA | GLY | 480 | −7.546 | 27.278 | −5.094 | 0.00 | 0.00 | 0.105 | 9.40 | 4.00 |
| ATOM | 2524 | HA1 | GLY | 480 | −7.253 | 27.724 | −4.144 | 0.00 | 0.00 | 0.053 | 0.00 | 0.00 |
| ATOM | 2525 | HA2 | GLY | 480 | −6.939 | 27.705 | −5.893 | 0.00 | 0.00 | 0.053 | 0.00 | 0.00 |
| ATOM | 2526 | C | GLY | 480 | −7.313 | 25.796 | −5.036 | 0.00 | 0.00 | 0.396 | 9.82 | 4.00 |
| ATOM | 2527 | O | GLY | 480 | −6.307 | 25.305 | −5.546 | 0.00 | 0.00 | −0.396 | 8.17 | −17.40 |
| ATOM | 2528 | N | SER | 481 | −8.235 | 25.042 | −4.409 | 0.00 | 0.00 | −0.650 | 9.00 | −17.40 |
| ATOM | 2529 | HN | SER | 481 | −9.070 | 25.498 | −4.015 | 0.00 | 0.00 | 0.439 | 0.00 | 0.00 |
| ATOM | 2530 | CA | SER | 481 | −8.088 | 23.622 | −4.273 | 0.00 | 0.00 | 0.158 | 9.40 | 4.00 |
| ATOM | 2531 | HA | SER | 481 | −7.129 | 23.411 | −3.801 | 0.00 | 0.00 | 0.053 | 0.00 | 0.00 |
| ATOM | 2532 | C | SER | 481 | −8.140 | 23.000 | −5.628 | 0.00 | 0.00 | 0.396 | 9.82 | 4.00 |
| ATOM | 2533 | O | SER | 481 | −7.439 | 22.028 | −5.896 | 0.00 | 0.00 | −0.396 | 8.17 | −17.40 |
| ATOM | 2534 | CB | SER | 481 | −9.207 | 22.981 | −3.429 | 0.00 | 0.00 | 0.007 | 12.77 | 4.00 |
| ATOM | 2535 | HB1 | SER | 481 | −9.148 | 21.893 | −3.471 | 0.00 | 0.00 | 0.053 | 0.00 | 0.00 |
| ATOM | 2536 | HB2 | SER | 481 | −10.188 | 23.282 | −3.796 | 0.00 | 0.00 | 0.053 | 0.00 | 0.00 |
| ATOM | 2537 | OG | SER | 481 | −9.102 | 23.378 | −2.068 | 0.00 | 0.00 | −0.537 | 11.04 | −17.40 |
| ATOM | 2538 | HG | SER | 481 | −8.805 | 24.363 | −2.019 | 0.00 | 0.00 | 0.424 | 0.00 | 0.00 |
| ATOM | 2539 | N | SER | 482 | −8.971 | 23.550 | −6.529 | 0.00 | 0.00 | −0.650 | 9.00 | −17.40 |
| ATOM | 2540 | HN | SER | 482 | −9.479 | 24.415 | −6.297 | 0.00 | 0.00 | 0.439 | 0.00 | 0.00 |
| ATOM | 2541 | CA | SER | 482 | −9.153 | 22.937 | −7.812 | 0.00 | 0.00 | 0.158 | 9.40 | 4.00 |
| ATOM | 2542 | HA | SER | 482 | −9.577 | 21.943 | −7.668 | 0.00 | 0.00 | 0.053 | 0.00 | 0.00 |
| ATOM | 2543 | C | SER | 482 | −7.828 | 22.838 | −8.488 | 0.00 | 0.00 | 0.396 | 9.82 | 4.00 |
| ATOM | 2544 | O | SER | 482 | −7.469 | 21.793 | −9.026 | 0.00 | 0.00 | −0.396 | 8.17 | −17.40 |
| ATOM | 2545 | CB | SER | 482 | −10.065 | 23.751 | −8.744 | 0.00 | 0.00 | 0.007 | 12.77 | 4.00 |
| ATOM | 2546 | HB1 | SER | 482 | −10.998 | 23.995 | −8.236 | 0.00 | 0.00 | 0.053 | 0.00 | 0.00 |
| ATOM | 2547 | HB2 | SER | 482 | −10.293 | 23.176 | −9.641 | 0.00 | 0.00 | 0.053 | 0.00 | 0.00 |
| ATOM | 2548 | OG | SER | 482 | −9.421 | 24.959 | −9.125 | 0.00 | 0.00 | −0.537 | 11.04 | −17.40 |
| ATOM | 2549 | HG | SER | 482 | −8.440 | 24.939 | −8.812 | 0.00 | 0.00 | 0.424 | 0.00 | 0.00 |
| ATOM | 2550 | N | THR | 483 | −7.047 | 23.928 | −8.449 | 0.00 | 0.00 | −0.650 | 9.00 | −17.40 |
| ATOM | 2551 | HN | THR | 483 | −7.358 | 24.770 | −7.945 | 0.00 | 0.00 | 0.439 | 0.00 | 0.00 |
| ATOM | 2552 | CA | THR | 483 | −5.781 | 23.914 | −9.111 | 0.00 | 0.00 | 0.158 | 9.40 | 4.00 |
| ATOM | 2553 | HA | THR | 483 | −5.971 | 23.731 | −10.169 | 0.00 | 0.00 | 0.053 | 0.00 | 0.00 |
| ATOM | 2554 | C | THR | 483 | −4.974 | 22.816 | −8.500 | 0.00 | 0.00 | 0.396 | 9.82 | 4.00 |
| ATOM | 2555 | O | THR | 483 | −4.267 | 22.085 | −9.192 | 0.00 | 0.00 | −0.396 | 8.17 | −17.40 |
| ATOM | 2556 | CB | THR | 483 | −5.031 | 25.199 | −8.925 | 0.00 | 0.00 | 0.060 | 9.40 | 4.00 |
| ATOM | 2557 | HB | THR | 483 | −4.857 | 25.354 | −7.860 | 0.00 | 0.00 | 0.053 | 0.00 | 0.00 |
| ATOM | 2558 | OG1 | THR | 483 | −5.795 | 26.283 | −9.434 | 0.00 | 0.00 | −0.537 | 11.04 | −17.40 |
| ATOM | 2559 | HG1 | THR | 483 | −5.239 | 27.149 | −9.385 | 0.00 | 0.00 | 0.424 | 0.00 | 0.00 |
| ATOM | 2560 | CG2 | THR | 483 | −3.691 | 25.104 | −9.670 | 0.00 | 0.00 | −0.159 | 16.15 | 4.00 |
| ATOM | 2561 | HG21 | THR | 483 | −3.137 | 26.034 | −9.542 | 0.00 | 0.00 | 0.053 | 0.00 | 0.00 |
| ATOM | 2562 | HG22 | THR | 483 | −3.109 | 24.276 | −9.267 | 0.00 | 0.00 | 0.053 | 0.00 | 0.00 |
| ATOM | 2563 | HG23 | THR | 483 | −3.876 | 24.935 | −10.731 | 0.00 | 0.00 | 0.053 | 0.00 | 0.00 |
| ATOM | 2564 | N | LYS | 484 | −5.079 | 22.671 | −7.171 | 0.00 | 0.00 | −0.650 | 9.00 | −17.40 |
| ATOM | 2565 | HN | LYS | 484 | −5.719 | 23.285 | −6.647 | 0.00 | 0.00 | 0.439 | 0.00 | 0.00 |
| ATOM | 2566 | CA | LYS | 484 | −4.323 | 21.685 | −6.464 | 0.00 | 0.00 | 0.158 | 9.40 | 4.00 |
| ATOM | 2567 | HA | LYS | 484 | −3.265 | 21.851 | −6.667 | 0.00 | 0.00 | 0.053 | 0.00 | 0.00 |
| ATOM | 2568 | C | LYS | 484 | −4.742 | 20.335 | −6.945 | 0.00 | 0.00 | 0.396 | 9.82 | 4.00 |
| ATOM | 2569 | O | LYS | 484 | −3.915 | 19.494 | −7.288 | 0.00 | 0.00 | −0.396 | 8.17 | −17.40 |
| ATOM | 2570 | CB | LYS | 484 | −4.621 | 21.725 | −4.957 | 0.00 | 0.00 | −0.106 | 12.77 | 4.00 |
| ATOM | 2571 | HB1 | LYS | 484 | −3.988 | 21.052 | −4.378 | 0.00 | 0.00 | 0.053 | 0.00 | 0.00 |
| ATOM | 2572 | HB2 | LYS | 484 | −5.646 | 21.443 | −4.716 | 0.00 | 0.00 | 0.053 | 0.00 | 0.00 |
| ATOM | 2573 | CG | LYS | 484 | −4.420 | 23.110 | −4.343 | 0.00 | 0.00 | −0.106 | 12.77 | 4.00 |
| ATOM | 2574 | HG1 | LYS | 484 | −4.637 | 23.149 | −3.276 | 0.00 | 0.00 | 0.053 | 0.00 | 0.00 |
| ATOM | 2575 | HG2 | LYS | 484 | −5.050 | 23.880 | −4.789 | 0.00 | 0.00 | 0.053 | 0.00 | 0.00 |
| ATOM | 2576 | CD | LYS | 484 | −2.992 | 23.636 | −4.478 | 0.00 | 0.00 | −0.106 | 12.77 | 4.00 |
| ATOM | 2577 | HD1 | LYS | 484 | −2.924 | 24.722 | −4.416 | 0.00 | 0.00 | 0.053 | 0.00 | 0.00 |
| ATOM | 2578 | HD2 | LYS | 484 | −2.522 | 23.371 | −5.425 | 0.00 | 0.00 | 0.053 | 0.00 | 0.00 |
| ATOM | 2579 | CE | LYS | 484 | −2.048 | 23.110 | −3.397 | 0.00 | 0.00 | 0.099 | 12.77 | 4.00 |
| ATOM | 2580 | HE1 | LYS | 484 | −1.019 | 23.367 | −3.649 | 0.00 | 0.00 | 0.053 | 0.00 | 0.00 |
| ATOM | 2581 | HE2 | LYS | 484 | −2.139 | 22.026 | −3.324 | 0.00 | 0.00 | 0.053 | 0.00 | 0.00 |
| ATOM | 2582 | NZ | LYS | 484 | −2.395 | 23.716 | −2.092 | 0.00 | 0.00 | −0.045 | 13.25 | −39.20 |
| ATOM | 2583 | HZ1 | LYS | 484 | −1.757 | 23.359 | −1.367 | 0.00 | 0.00 | 0.280 | 0.00 | 0.00 |
| ATOM | 2584 | HZ2 | LYS | 484 | −2.305 | 24.740 | −2.154 | 0.00 | 0.00 | 0.280 | 0.00 | 0.00 |
| ATOM | 2585 | HZ3 | LYS | 484 | −3.365 | 23.471 | −1.846 | 0.00 | 0.00 | 0.280 | 0.00 | 0.00 |
| ATOM | 2586 | N | ASN | 485 | −6.063 | 20.108 | −7.015 | 0.00 | 0.00 | −0.650 | 9.00 | −17.40 |
| ATOM | 2587 | HN | ASN | 485 | −6.720 | 20.869 | −6.790 | 0.00 | 0.00 | 0.439 | 0.00 | 0.00 |
| ATOM | 2588 | CA | ASN | 485 | −6.573 | 18.829 | −7.396 | 0.00 | 0.00 | 0.158 | 9.40 | 4.00 |
| ATOM | 2589 | HA | ASN | 485 | −6.178 | 18.096 | −6.693 | 0.00 | 0.00 | 0.053 | 0.00 | 0.00 |
| ATOM | 2590 | C | ASN | 485 | −6.107 | 18.556 | −8.784 | 0.00 | 0.00 | 0.396 | 9.82 | 4.00 |
| ATOM | 2591 | O | ASN | 485 | −5.709 | 17.438 | −9.109 | 0.00 | 0.00 | −0.396 | 8.17 | −17.40 |
| ATOM | 2592 | CB | ASN | 485 | −8.112 | 18.787 | −7.379 | 0.00 | 0.00 | −0.106 | 12.77 | 4.00 |
| ATOM | 2593 | HB1 | ASN | 485 | −8.486 | 19.345 | −8.237 | 0.00 | 0.00 | 0.053 | 0.00 | 0.00 |
| ATOM | 2594 | HB2 | ASN | 485 | −8.463 | 19.240 | −6.452 | 0.00 | 0.00 | 0.053 | 0.00 | 0.00 |
| ATOM | 2595 | CG | ASN | 485 | −8.561 | 17.335 | −7.459 | 0.00 | 0.00 | 0.396 | 9.82 | 4.00 |
| ATOM | 2596 | OD1 | ASN | 485 | −8.471 | 16.697 | −8.507 | 0.00 | 0.00 | −0.396 | 8.17 | −17.40 |
| ATOM | 2597 | ND2 | ASN | 485 | −9.057 | 16.795 | −6.315 | 0.00 | 0.00 | −0.879 | 13.25 | −17.40 |
| ATOM | 2598 | HD21 | ASN | 485 | −9.115 | 17.365 | −5.459 | 0.00 | 0.00 | 0.439 | 0.00 | 0.00 |
| ATOM | 2599 | HD22 | ASN | 485 | −9.375 | 15.816 | −6.304 | 0.00 | 0.00 | 0.439 | 0.00 | 0.00 |
| ATOM | 2600 | N | ILE | 486 | −6.116 | 19.597 | −9.635 | 0.00 | 0.00 | −0.650 | 9.00 | −17.40 |
| ATOM | 2601 | HN | ILE | 486 | −6.399 | 20.528 | −9.296 | 0.00 | 0.00 | 0.439 | 0.00 | 0.00 |
| ATOM | 2602 | CA | ILE | 486 | −5.738 | 19.429 | −11.005 | 0.00 | 0.00 | 0.158 | 9.40 | 4.00 |
| ATOM | 2603 | HA | ILE | 486 | −6.405 | 18.695 | −11.457 | 0.00 | 0.00 | 0.053 | 0.00 | 0.00 |
| ATOM | 2604 | C | ILE | 486 | −4.326 | 18.951 | −11.046 | 0.00 | 0.00 | 0.396 | 9.82 | 4.00 |
| ATOM | 2605 | O | ILE | 486 | −3.371 | 19.702 | −10.857 | 0.00 | 0.00 | −0.396 | 8.17 | −17.40 |
| ATOM | 2606 | CB | ILE | 486 | −5.804 | 20.702 | −11.792 | 0.00 | 0.00 | −0.053 | 9.40 | 4.00 |
| ATOM | 2607 | HB | ILE | 486 | −5.204 | 21.452 | −11.277 | 0.00 | 0.00 | 0.053 | 0.00 | 0.00 |
| ATOM | 2608 | CG1 | ILE | 486 | −7.240 | 21.251 | −11.791 | 0.00 | 0.00 | −0.106 | 12.77 | 4.00 |
| ATOM | 2609 | HG11 | ILE | 486 | −7.699 | 21.265 | −10.802 | 0.00 | 0.00 | 0.053 | 0.00 | 0.00 |
| ATOM | 2610 | HG12 | ILE | 486 | −7.925 | 20.677 | −12.415 | 0.00 | 0.00 | 0.053 | 0.00 | 0.00 |
| ATOM | 2611 | CG2 | ILE | 486 | −5.243 | 20.427 | −13.196 | 0.00 | 0.00 | −0.159 | 16.15 | 4.00 |
| ATOM | 2612 | HG21 | ILE | 486 | −5.281 | 21.341 | −13.788 | 0.00 | 0.00 | 0.053 | 0.00 | 0.00 |
| ATOM | 2613 | HG22 | ILE | 486 | −4.210 | 20.090 | −13.115 | 0.00 | 0.00 | 0.053 | 0.00 | 0.00 |
| ATOM | 2614 | HG23 | ILE | 486 | −5.840 | 19.655 | −13.682 | 0.00 | 0.00 | 0.053 | 0.00 | 0.00 |
| ATOM | 2615 | CD1 | ILE | 486 | −7.343 | 22.687 | −12.300 | 0.00 | 0.00 | −0.159 | 16.15 | 4.00 |
| ATOM | 2616 | HD11 | ILE | 486 | −8.384 | 23.009 | −12.272 | 0.00 | 0.00 | 0.053 | 0.00 | 0.00 |
| ATOM | 2617 | HD12 | ILE | 486 | −6.743 | 23.341 | −11.667 | 0.00 | 0.00 | 0.053 | 0.00 | 0.00 |
| ATOM | 2618 | HD13 | ILE | 486 | −6.975 | 22.737 | −13.325 | 0.00 | 0.00 | 0.053 | 0.00 | 0.00 |
| ATOM | 2619 | N | PRO | 487 | −4.207 | 17.684 | −11.301 | 0.00 | 0.00 | −0.422 | 9.00 | −17.40 |
| ATOM | 2620 | CA | PRO | 487 | −2.931 | 17.040 | −11.373 | 0.00 | 0.00 | 0.158 | 9.40 | 4.00 |
| ATOM | 2621 | HA | PRO | 487 | −2.466 | 17.109 | −10.389 | 0.00 | 0.00 | 0.053 | 0.00 | 0.00 |
| ATOM | 2622 | CD | PRO | 487 | −5.205 | 16.985 | −12.087 | 0.00 | 0.00 | 0.105 | 12.77 | 4.00 |
| ATOM | 2623 | HD1 | PRO | 487 | −5.569 | 17.718 | −12.807 | 0.00 | 0.00 | 0.053 | 0.00 | 0.00 |
| ATOM | 2624 | HD2 | PRO | 487 | −5.964 | 16.669 | −11.372 | 0.00 | 0.00 | 0.053 | 0.00 | 0.00 |
| ATOM | 2625 | C | PRO | 487 | −2.123 | 17.747 | −12.394 | 0.00 | 0.00 | 0.396 | 9.82 | 4.00 |
| ATOM | 2626 | O | PRO | 487 | −0.898 | 17.746 | −12.276 | 0.00 | 0.00 | −0.396 | 8.17 | −17.40 |
| ATOM | 2627 | CB | PRO | 487 | −3.214 | 15.623 | −11.853 | 0.00 | 0.00 | −0.106 | 12.77 | 4.00 |
| ATOM | 2628 | HB1 | PRO | 487 | −3.406 | 14.954 | −11.015 | 0.00 | 0.00 | 0.053 | 0.00 | 0.00 |
| ATOM | 2629 | HB2 | PRO | 487 | −2.369 | 15.220 | −12.412 | 0.00 | 0.00 | 0.053 | 0.00 | 0.00 |
| ATOM | 2630 | CG | PRO | 487 | −4.457 | 15.810 | −12.740 | 0.00 | 0.00 | −0.106 | 12.77 | 4.00 |
| ATOM | 2631 | HG1 | PRO | 487 | −5.065 | 14.905 | −12.753 | 0.00 | 0.00 | 0.053 | 0.00 | 0.00 |
| ATOM | 2632 | HG2 | PRO | 487 | −4.171 | 16.038 | −13.767 | 0.00 | 0.00 | 0.053 | 0.00 | 0.00 |
| ATOM | 2633 | N | SER | 488 | −2.776 | 18.321 | −13.418 | 0.00 | 0.00 | −0.650 | 9.00 | −17.40 |
| ATOM | 2634 | HN | SER | 488 | −3.803 | 18.279 | −13.479 | 0.00 | 0.00 | 0.439 | 0.00 | 0.00 |
| ATOM | 2635 | CA | SER | 488 | −2.012 | 18.989 | −14.418 | 0.00 | 0.00 | 0.158 | 9.40 | 4.00 |
| ATOM | 2636 | HA | SER | 488 | −1.360 | 18.220 | −14.833 | 0.00 | 0.00 | 0.053 | 0.00 | 0.00 |
| ATOM | 2637 | C | SER | 488 | −1.285 | 20.055 | −13.693 | 0.00 | 0.00 | 0.396 | 9.82 | 4.00 |
| ATOM | 2638 | O | SER | 488 | −0.065 | 20.165 | −13.814 | 0.00 | 0.00 | −0.396 | 8.17 | −17.40 |
| ATOM | 2639 | CB | SER | 488 | −2.876 | 19.662 | −15.496 | 0.00 | 0.00 | 0.007 | 12.77 | 4.00 |
| ATOM | 2640 | HB1 | SER | 488 | −3.532 | 20.405 | −15.042 | 0.00 | 0.00 | 0.053 | 0.00 | 0.00 |
| ATOM | 2641 | HB2 | SER | 488 | −3.488 | 18.918 | −16.006 | 0.00 | 0.00 | 0.053 | 0.00 | 0.00 |
| ATOM | 2642 | OG | SER | 488 | −2.050 | 20.307 | −16.453 | 0.00 | 0.00 | −0.537 | 11.04 | −17.40 |
| ATOM | 2643 | HG | SER | 488 | −1.409 | 20.954 | −15.973 | 0.00 | 0.00 | 0.424 | 0.00 | 0.00 |
| ATOM | 2644 | N | TYR | 489 | −2.025 | 20.795 | −12.837 | 0.00 | 0.00 | −0.650 | 9.00 | −17.40 |
| ATOM | 2645 | HN | TYR | 489 | −3.028 | 20.587 | −12.731 | 0.00 | 0.00 | 0.439 | 0.00 | 0.00 |
| ATOM | 2646 | CA | TYR | 489 | −1.448 | 21.860 | −12.074 | 0.00 | 0.00 | 0.158 | 9.40 | 4.00 |
| ATOM | 2647 | HA | TYR | 489 | −2.291 | 22.377 | −11.615 | 0.00 | 0.00 | 0.053 | 0.00 | 0.00 |
| ATOM | 2648 | C | TYR | 489 | −0.710 | 22.699 | −13.060 | 0.00 | 0.00 | 0.396 | 9.82 | 4.00 |
| ATOM | 2649 | O | TYR | 489 | −1.052 | 22.716 | −14.244 | 0.00 | 0.00 | −0.396 | 8.17 | −17.40 |
| ATOM | 2650 | CB | TYR | 489 | −0.420 | 21.293 | −11.086 | 0.00 | 0.00 | −0.106 | 12.77 | 4.00 |
| ATOM | 2651 | HB1 | TYR | 489 | 0.549 | 21.199 | −11.575 | 0.00 | 0.00 | 0.053 | 0.00 | 0.00 |
| ATOM | 2652 | HB2 | TYR | 489 | −0.743 | 20.311 | −10.741 | 0.00 | 0.00 | 0.053 | 0.00 | 0.00 |
| ATOM | 2653 | CG | TYR | 489 | −0.291 | 22.199 | −9.922 | 0.00 | 0.00 | 0.000 | 7.26 | 0.60 |
| ATOM | 2654 | CD1 | TYR | 489 | −1.257 | 22.125 | −8.949 | 0.00 | 0.00 | −0.127 | 10.80 | 0.60 |
| ATOM | 2655 | HD1 | TYR | 489 | −2.082 | 21.422 | −9.062 | 0.00 | 0.00 | 0.127 | 0.00 | 0.00 |
| ATOM | 2656 | CD2 | TYR | 489 | 0.757 | 23.078 | −9.774 | 0.00 | 0.00 | −0.127 | 10.80 | 0.60 |
| ATOM | 2657 | HD2 | TYR | 489 | 1.533 | 23.137 | −10.537 | 0.00 | 0.00 | 0.127 | 0.00 | 0.00 |
| ATOM | 2658 | CE1 | TYR | 489 | −1.196 | 22.924 | −7.838 | 0.00 | 0.00 | −0.127 | 10.80 | 0.60 |
| ATOM | 2659 | HE1 | TYR | 489 | −1.970 | 22.859 | −7.073 | 0.00 | 0.00 | 0.127 | 0.00 | 0.00 |
| ATOM | 2660 | CE2 | TYR | 489 | 0.822 | 23.883 | −8.660 | 0.00 | 0.00 | −0.127 | 10.80 | 0.60 |
| ATOM | 2661 | HE2 | TYR | 489 | 1.649 | 24.583 | −8.542 | 0.00 | 0.00 | 0.127 | 0.00 | 0.00 |
| ATOM | 2662 | CZ | TYR | 489 | −0.156 | 23.806 | −7.694 | 0.00 | 0.00 | 0.027 | 7.26 | 0.60 |
| ATOM | 2663 | OH | TYR | 489 | −0.097 | 24.626 | −6.548 | 0.00 | 0.00 | −0.451 | 10.94 | −17.40 |
| ATOM | 2664 | HH | TYR | 489 | 0.482 | 25.454 | −6.747 | 0.00 | 0.00 | 0.424 | 0.00 | 0.00 |
| ATOM | 2665 | N | VAL | 490 | 0.268 | 23.499 | −12.604 | 0.00 | 0.00 | −0.650 | 9.00 | −17.40 |
| ATOM | 2666 | HN | VAL | 490 | 0.442 | 23.668 | −11.603 | 0.00 | 0.00 | 0.439 | 0.00 | 0.00 |
| ATOM | 2667 | CA | VAL | 490 | 1.041 | 24.090 | −13.645 | 0.00 | 0.00 | 0.158 | 9.40 | 4.00 |
| ATOM | 2668 | HA | VAL | 490 | 0.411 | 24.640 | −14.344 | 0.00 | 0.00 | 0.053 | 0.00 | 0.00 |
| ATOM | 2669 | C | VAL | 490 | 1.680 | 22.891 | −14.277 | 0.00 | 0.00 | 0.396 | 9.82 | 4.00 |
| ATOM | 2670 | O | VAL | 490 | 2.373 | 22.113 | −13.624 | 0.00 | 0.00 | −0.396 | 8.17 | −17.40 |
| ATOM | 2671 | CB | VAL | 490 | 2.057 | 25.081 | −13.165 | 0.00 | 0.00 | −0.053 | 9.40 | 4.00 |
| ATOM | 2672 | HB | VAL | 490 | 2.698 | 24.600 | −12.426 | 0.00 | 0.00 | 0.053 | 0.00 | 0.00 |
| ATOM | 2673 | CG1 | VAL | 490 | 2.898 | 25.553 | −14.364 | 0.00 | 0.00 | −0.159 | 16.15 | 4.00 |
| ATOM | 2674 | HG11 | VAL | 490 | 3.641 | 26.275 | −14.025 | 0.00 | 0.00 | 0.053 | 0.00 | 0.00 |
| ATOM | 2675 | HG12 | VAL | 490 | 3.402 | 24.698 | −14.814 | 0.00 | 0.00 | 0.053 | 0.00 | 0.00 |
| ATOM | 2676 | HG13 | VAL | 490 | 2.248 | 26.021 | −15.103 | 0.00 | 0.00 | 0.053 | 0.00 | 0.00 |
| ATOM | 2677 | CG2 | VAL | 490 | 1.289 | 26.226 | −12.478 | 0.00 | 0.00 | −0.159 | 16.15 | 4.00 |
| ATOM | 2678 | HG21 | VAL | 490 | 1.996 | 26.971 | −12.113 | 0.00 | 0.00 | 0.053 | 0.00 | 0.00 |
| ATOM | 2679 | HG22 | VAL | 490 | 0.610 | 26.690 | −13.194 | 0.00 | 0.00 | 0.053 | 0.00 | 0.00 |
| ATOM | 2680 | HG23 | VAL | 490 | 0.716 | 25.829 | −11.640 | 0.00 | 0.00 | 0.053 | 0.00 | 0.00 |
| ATOM | 2681 | N | LYS | 491 | 1.408 | 22.708 | −15.580 | 0.00 | 0.00 | −0.650 | 9.00 | −17.40 |
| ATOM | 2682 | HN | LYS | 491 | 0.944 | 23.478 | −16.083 | 0.00 | 0.00 | 0.439 | 0.00 | 0.00 |
| ATOM | 2683 | CA | LYS | 491 | 1.715 | 21.520 | −16.324 | 0.00 | 0.00 | 0.158 | 9.40 | 4.00 |
| ATOM | 2684 | HA | LYS | 491 | 1.081 | 20.712 | −15.959 | 0.00 | 0.00 | 0.053 | 0.00 | 0.00 |
| ATOM | 2685 | C | LYS | 491 | 3.139 | 21.075 | −16.200 | 0.00 | 0.00 | 0.396 | 9.82 | 4.00 |
| ATOM | 2686 | O | LYS | 491 | 3.988 | 21.759 | −15.631 | 0.00 | 0.00 | −0.396 | 8.17 | −17.40 |
| ATOM | 2687 | CB | LYS | 491 | 1.342 | 21.656 | −17.807 | 0.00 | 0.00 | −0.106 | 12.77 | 4.00 |
| ATOM | 2688 | HB1 | LYS | 491 | 1.766 | 20.859 | −18.418 | 0.00 | 0.00 | 0.053 | 0.00 | 0.00 |
| ATOM | 2689 | HB2 | LYS | 491 | 1.692 | 22.593 | −18.241 | 0.00 | 0.00 | 0.053 | 0.00 | 0.00 |
| ATOM | 2690 | CG | LYS | 491 | −0.170 | 21.615 | −18.036 | 0.00 | 0.00 | −0.106 | 12.77 | 4.00 |
| ATOM | 2691 | HG1 | LYS | 491 | −0.561 | 20.718 | −17.555 | 0.00 | 0.00 | 0.053 | 0.00 | 0.00 |
| ATOM | 2692 | HG2 | LYS | 491 | −0.349 | 21.588 | −19.111 | 0.00 | 0.00 | 0.053 | 0.00 | 0.00 |
| ATOM | 2693 | CD | LYS | 491 | −0.945 | 22.805 | −17.476 | 0.00 | 0.00 | −0.106 | 12.77 | 4.00 |
| ATOM | 2694 | HD1 | LYS | 491 | −0.593 | 23.754 | −17.880 | 0.00 | 0.00 | 0.053 | 0.00 | 0.00 |
| ATOM | 2695 | HD2 | LYS | 491 | −0.861 | 22.879 | −16.392 | 0.00 | 0.00 | 0.053 | 0.00 | 0.00 |
| ATOM | 2696 | CE | LYS | 491 | −2.440 | 22.724 | −17.793 | 0.00 | 0.00 | 0.099 | 12.77 | 4.00 |
| ATOM | 2697 | HE1 | LYS | 491 | −2.946 | 23.618 | −17.428 | 0.00 | 0.00 | 0.053 | 0.00 | 0.00 |
| ATOM | 2698 | HE2 | LYS | 491 | −2.875 | 21.848 | −17.311 | 0.00 | 0.00 | 0.053 | 0.00 | 0.00 |
| ATOM | 2699 | NZ | LYS | 491 | −2.640 | 22.619 | −19.256 | 0.00 | 0.00 | −0.045 | 13.25 | −39.20 |
| ATOM | 2700 | HZ1 | LYS | 491 | −3.648 | 22.565 | −19.462 | 0.00 | 0.00 | 0.280 | 0.00 | 0.00 |
| ATOM | 2701 | HZ2 | LYS | 491 | −2.239 | 23.448 | −19.718 | 0.00 | 0.00 | 0.280 | 0.00 | 0.00 |
| ATOM | 2702 | HZ3 | LYS | 491 | −2.172 | 21.771 | −19.607 | 0.00 | 0.00 | 0.280 | 0.00 | 0.00 |
| ATOM | 2703 | N | ASN | 492 | 3.399 | 19.865 | −16.755 | 0.00 | 0.00 | −0.650 | 9.00 | −17.40 |
| ATOM | 2704 | HN | ASN | 492 | 2.617 | 19.385 | −17.223 | 0.00 | 0.00 | 0.439 | 0.00 | 0.00 |
| ATOM | 2705 | CA | ASN | 492 | 4.671 | 19.192 | −16.748 | 0.00 | 0.00 | 0.158 | 9.40 | 4.00 |
| ATOM | 2706 | HA | ASN | 492 | 5.361 | 19.867 | −16.242 | 0.00 | 0.00 | 0.053 | 0.00 | 0.00 |
| ATOM | 2707 | C | ASN | 492 | 5.026 | 18.969 | −18.196 | 0.00 | 0.00 | 0.396 | 9.82 | 4.00 |
| ATOM | 2708 | O | ASN | 492 | 4.132 | 18.867 | −19.033 | 0.00 | 0.00 | −0.396 | 8.17 | −17.40 |
| ATOM | 2709 | CB | ASN | 492 | 4.535 | 17.774 | −16.204 | 0.00 | 0.00 | −0.106 | 12.77 | 4.00 |
| ATOM | 2710 | HB1 | ASN | 492 | 5.500 | 17.524 | −15.764 | 0.00 | 0.00 | 0.053 | 0.00 | 0.00 |
| ATOM | 2711 | HB2 | ASN | 492 | 4.289 | 17.143 | −17.058 | 0.00 | 0.00 | 0.053 | 0.00 | 0.00 |
| ATOM | 2712 | CG | ASN | 492 | 3.422 | 17.821 | −15.177 | 0.00 | 0.00 | 0.396 | 9.82 | 4.00 |
| ATOM | 2713 | OD1 | ASN | 492 | 3.509 | 18.436 | −14.120 | 0.00 | 0.00 | −0.396 | 8.17 | −17.40 |
| ATOM | 2714 | ND2 | ASN | 492 | 2.296 | 17.149 | −15.534 | 0.00 | 0.00 | −0.879 | 13.25 | −17.40 |
| ATOM | 2715 | HD21 | ASN | 492 | 2.260 | 16.643 | −16.431 | 0.00 | 0.00 | 0.439 | 0.00 | 0.00 |
| ATOM | 2716 | HD22 | ASN | 492 | 1.478 | 17.145 | −14.908 | 0.00 | 0.00 | 0.439 | 0.00 | 0.00 |
| ATOM | 2717 | N | HIS | 493 | 6.333 | 18.905 | −18.550 | 0.00 | 0.00 | −0.650 | 9.00 | −17.40 |
| ATOM | 2718 | HN | HIS | 493 | 7.074 | 19.059 | −17.851 | 0.00 | 0.00 | 0.439 | 0.00 | 0.00 |
| ATOM | 2719 | CA | HIS | 493 | 6.666 | 18.617 | −19.922 | 0.00 | 0.00 | 0.158 | 9.40 | 4.00 |
| ATOM | 2720 | HA | HIS | 493 | 6.337 | 17.600 | −20.137 | 0.00 | 0.00 | 0.053 | 0.00 | 0.00 |
| ATOM | 2721 | C | HIS | 493 | 8.157 | 18.747 | −20.068 | 0.00 | 0.00 | 0.396 | 9.82 | 4.00 |
| ATOM | 2722 | O | HIS | 493 | 8.846 | 19.118 | −19.120 | 0.00 | 0.00 | −0.396 | 8.17 | −17.40 |
| ATOM | 2723 | CB | HIS | 493 | 5.983 | 19.562 | −20.948 | 0.00 | 0.00 | −0.106 | 12.77 | 4.00 |
| ATOM | 2724 | HB1 | HIS | 493 | 5.214 | 20.127 | −20.422 | 0.00 | 0.00 | 0.053 | 0.00 | 0.00 |
| ATOM | 2725 | HB2 | HIS | 493 | 5.546 | 18.944 | −21.732 | 0.00 | 0.00 | 0.053 | 0.00 | 0.00 |
| ATOM | 2726 | CG | HIS | 493 | 6.882 | 20.563 | −21.624 | 0.00 | 0.00 | 0.241 | 7.26 | 0.60 |
| ATOM | 2727 | ND1 | HIS | 493 | 7.414 | 20.337 | −22.876 | 0.00 | 0.00 | −0.481 | 9.25 | −17.40 |
| ATOM | 2728 | CD2 | HIS | 493 | 7.369 | 21.771 | −21.230 | 0.00 | 0.00 | −0.177 | 10.80 | 0.60 |
| ATOM | 2729 | HD2 | HIS | 493 | 7.143 | 22.250 | −20.278 | 0.00 | 0.00 | 0.127 | 0.00 | 0.00 |
| ATOM | 2730 | CE1 | HIS | 493 | 8.187 | 21.410 | −23.175 | 0.00 | 0.00 | 0.064 | 10.80 | 0.60 |
| ATOM | 2731 | HE1 | HIS | 493 | 8.740 | 21.520 | −24.108 | 0.00 | 0.00 | 0.127 | 0.00 | 0.00 |
| ATOM | 2732 | NE2 | HIS | 493 | 8.192 | 22.307 | −22.206 | 0.00 | 0.00 | −0.292 | 9.25 | −17.40 |
| ATOM | 2733 | HE2 | HIS | 493 | 8.696 | 23.205 | −22.182 | 0.00 | 0.00 | 0.393 | 0.00 | 0.00 |
| ATOM | 2734 | N | GLY | 494 | 8.696 | 18.406 | −21.262 | 0.00 | 0.00 | −0.650 | 9.00 | −17.40 |
| ATOM | 2735 | HN | GLY | 494 | 8.079 | 18.030 | −21.996 | 0.00 | 0.00 | 0.439 | 0.00 | 0.00 |
| ATOM | 2736 | CA | GLY | 494 | 10.095 | 18.549 | −21.543 | 0.00 | 0.00 | 0.105 | 9.40 | 4.00 |
| ATOM | 2737 | HA1 | GLY | 494 | 10.539 | 17.563 | −21.407 | 0.00 | 0.00 | 0.053 | 0.00 | 0.00 |
| ATOM | 2738 | HA2 | GLY | 494 | 10.479 | 19.279 | −20.830 | 0.00 | 0.00 | 0.053 | 0.00 | 0.00 |
| ATOM | 2739 | C | GLY | 494 | 10.195 | 19.028 | −22.958 | 0.00 | 0.00 | 0.396 | 9.82 | 4.00 |
| ATOM | 2740 | O | GLY | 494 | 9.335 | 18.724 | −23.783 | 0.00 | 0.00 | −0.396 | 8.17 | −17.40 |
| ATOM | 2741 | N | ILE | 495 | 11.248 | 19.808 | −23.277 | 0.00 | 0.00 | −0.650 | 9.00 | −17.40 |
| ATOM | 2742 | HN | ILE | 495 | 11.943 | 20.049 | −22.557 | 0.00 | 0.00 | 0.439 | 0.00 | 0.00 |
| ATOM | 2743 | CA | ILE | 495 | 11.405 | 20.304 | −24.616 | 0.00 | 0.00 | 0.158 | 9.40 | 4.00 |
| ATOM | 2744 | HA | ILE | 495 | 10.661 | 19.814 | −25.245 | 0.00 | 0.00 | 0.053 | 0.00 | 0.00 |
| ATOM | 2745 | C | ILE | 495 | 12.792 | 19.973 | −25.070 | 0.00 | 0.00 | 0.396 | 9.82 | 4.00 |
| ATOM | 2746 | O | ILE | 495 | 13.749 | 20.122 | −24.315 | 0.00 | 0.00 | −0.396 | 8.17 | −17.40 |
| ATOM | 2747 | CB | ILE | 495 | 11.221 | 21.802 | −24.691 | 0.00 | 0.00 | −0.053 | 9.40 | 4.00 |
| ATOM | 2748 | HB | ILE | 495 | 10.253 | 22.046 | −24.253 | 0.00 | 0.00 | 0.053 | 0.00 | 0.00 |
| ATOM | 2749 | CG1 | ILE | 495 | 11.078 | 22.314 | −26.137 | 0.00 | 0.00 | −0.106 | 12.77 | 4.00 |
| ATOM | 2750 | HG11 | ILE | 495 | 10.818 | 23.372 | −26.101 | 0.00 | 0.00 | 0.053 | 0.00 | 0.00 |
| ATOM | 2751 | HG12 | ILE | 495 | 10.290 | 21.741 | −26.626 | 0.00 | 0.00 | 0.053 | 0.00 | 0.00 |
| ATOM | 2752 | CG2 | ILE | 495 | 12.363 | 22.463 | −23.899 | 0.00 | 0.00 | −0.159 | 16.15 | 4.00 |
| ATOM | 2753 | HG21 | ILE | 495 | 12.253 | 23.547 | −23.938 | 0.00 | 0.00 | 0.053 | 0.00 | 0.00 |
| ATOM | 2754 | HG22 | ILE | 495 | 12.326 | 22.131 | −22.861 | 0.00 | 0.00 | 0.053 | 0.00 | 0.00 |
| ATOM | 2755 | HG23 | ILE | 495 | 13.320 | 22.180 | −24.336 | 0.00 | 0.00 | 0.053 | 0.00 | 0.00 |
| ATOM | 2756 | CD1 | ILE | 495 | 12.342 | 22.177 | −26.980 | 0.00 | 0.00 | −0.159 | 16.15 | 4.00 |
| ATOM | 2757 | HD11 | ILE | 495 | 12.154 | 22.561 | −27.983 | 0.00 | 0.00 | 0.053 | 0.00 | 0.00 |
| ATOM | 2758 | HD12 | ILE | 495 | 13.150 | 22.746 | −26.519 | 0.00 | 0.00 | 0.053 | 0.00 | 0.00 |
| ATOM | 2759 | HD13 | ILE | 495 | 12.627 | 21.127 | −27.041 | 0.00 | 0.00 | 0.053 | 0.00 | 0.00 |
| ATOM | 2760 | N | LEU | 496 | 12.950 | 19.498 | −26.321 | 0.00 | 0.00 | −0.650 | 9.00 | −17.40 |
| ATOM | 2761 | HN | LEU | 496 | 12.139 | 19.349 | −26.938 | 0.00 | 0.00 | 0.439 | 0.00 | 0.00 |
| ATOM | 2762 | CA | LEU | 496 | 14.281 | 19.206 | −26.767 | 0.00 | 0.00 | 0.158 | 9.40 | 4.00 |
| ATOM | 2763 | HA | LEU | 496 | 14.811 | 18.743 | −25.934 | 0.00 | 0.00 | 0.053 | 0.00 | 0.00 |
| ATOM | 2764 | C | LEU | 496 | 14.914 | 20.499 | −27.165 | 0.00 | 0.00 | 0.396 | 9.82 | 4.00 |
| ATOM | 2765 | O | LEU | 496 | 14.878 | 20.879 | −28.334 | 0.00 | 0.00 | −0.396 | 8.17 | −17.40 |
| ATOM | 2766 | CB | LEU | 496 | 14.356 | 18.299 | −28.011 | 0.00 | 0.00 | −0.106 | 12.77 | 4.00 |
| ATOM | 2767 | HB1 | LEU | 496 | 15.385 | 18.307 | −28.371 | 0.00 | 0.00 | 0.053 | 0.00 | 0.00 |
| ATOM | 2768 | HB2 | LEU | 496 | 13.677 | 18.702 | −28.762 | 0.00 | 0.00 | 0.053 | 0.00 | 0.00 |
| ATOM | 2769 | CG | LEU | 496 | 13.963 | 16.824 | −27.787 | 0.00 | 0.00 | −0.053 | 9.40 | 4.00 |
| ATOM | 2770 | HG | LEU | 496 | 13.911 | 16.280 | −28.730 | 0.00 | 0.00 | 0.053 | 0.00 | 0.00 |
| ATOM | 2771 | CD1 | LEU | 496 | 14.984 | 16.104 | −26.892 | 0.00 | 0.00 | −0.159 | 16.15 | 4.00 |
| ATOM | 2772 | HD11 | LEU | 496 | 14.679 | 15.067 | −26.753 | 0.00 | 0.00 | 0.053 | 0.00 | 0.00 |
| ATOM | 2773 | HD12 | LEU | 496 | 15.966 | 16.133 | −27.364 | 0.00 | 0.00 | 0.053 | 0.00 | 0.00 |
| ATOM | 2774 | HD13 | LEU | 496 | 15.032 | 16.601 | −25.923 | 0.00 | 0.00 | 0.053 | 0.00 | 0.00 |
| ATOM | 2775 | CD2 | LEU | 496 | 12.517 | 16.687 | −27.287 | 0.00 | 0.00 | −0.159 | 16.15 | 4.00 |
| ATOM | 2776 | HD21 | LEU | 496 | 12.280 | 15.633 | −27.142 | 0.00 | 0.00 | 0.053 | 0.00 | 0.00 |
| ATOM | 2777 | HD22 | LEU | 496 | 12.407 | 17.217 | −26.341 | 0.00 | 0.00 | 0.053 | 0.00 | 0.00 |
| ATOM | 2778 | HD23 | LEU | 496 | 11.836 | 17.114 | −28.023 | 0.00 | 0.00 | 0.053 | 0.00 | 0.00 |
| ATOM | 2779 | N | SER | 497 | 15.507 | 21.230 | −26.199 | 0.00 | 0.00 | −0.650 | 9.00 | −17.40 |
| ATOM | 2780 | HN | SER | 497 | 15.494 | 20.925 | −25.215 | 0.00 | 0.00 | 0.439 | 0.00 | 0.00 |
| ATOM | 2781 | CA | SER | 497 | 16.155 | 22.446 | −26.592 | 0.00 | 0.00 | 0.158 | 9.40 | 4.00 |
| ATOM | 2782 | HA | SER | 497 | 16.934 | 22.208 | −27.316 | 0.00 | 0.00 | 0.053 | 0.00 | 0.00 |
| ATOM | 2783 | C | SER | 497 | 16.753 | 23.083 | −25.380 | 0.00 | 0.00 | 0.396 | 9.82 | 4.00 |
| ATOM | 2784 | O | SER | 497 | 16.077 | 23.275 | −24.370 | 0.00 | 0.00 | −0.396 | 8.17 | −17.40 |
| ATOM | 2785 | CB | SER | 497 | 15.200 | 23.475 | −27.219 | 0.00 | 0.00 | 0.007 | 12.77 | 4.00 |
| ATOM | 2786 | HB1 | SER | 497 | 14.422 | 23.743 | −26.504 | 0.00 | 0.00 | 0.053 | 0.00 | 0.00 |
| ATOM | 2787 | HB2 | SER | 497 | 14.734 | 23.054 | −28.110 | 0.00 | 0.00 | 0.053 | 0.00 | 0.00 |
| ATOM | 2788 | OG | SER | 497 | 15.919 | 24.644 | −27.581 | 0.00 | 0.00 | −0.537 | 11.04 | −17.40 |
| ATOM | 2789 | HG | SER | 497 | 16.893 | 24.563 | −27.255 | 0.00 | 0.00 | 0.424 | 0.00 | 0.00 |
| ATOM | 2790 | N | GLY | 498 | 18.050 | 23.438 | −25.472 | 0.00 | 0.00 | −0.650 | 9.00 | −17.40 |
| ATOM | 2791 | HN | GLY | 498 | 18.567 | 23.250 | −26.343 | 0.00 | 0.00 | 0.439 | 0.00 | 0.00 |
| ATOM | 2792 | CA | GLY | 498 | 18.719 | 24.073 | −24.376 | 0.00 | 0.00 | 0.105 | 9.40 | 4.00 |
| ATOM | 2793 | HA1 | GLY | 498 | 18.252 | 25.047 | −24.232 | 0.00 | 0.00 | 0.053 | 0.00 | 0.00 |
| ATOM | 2794 | HA2 | GLY | 498 | 19.770 | 24.169 | −24.649 | 0.00 | 0.00 | 0.053 | 0.00 | 0.00 |
| ATOM | 2795 | C | GLY | 498 | 18.542 | 23.200 | −23.183 | 0.00 | 0.00 | 0.396 | 9.82 | 4.00 |
| ATOM | 2796 | O | GLY | 498 | 18.502 | 21.976 | −23.292 | 0.00 | 0.00 | −0.396 | 8.17 | −17.40 |
| ATOM | 2797 | N | ARG | 499 | 18.435 | 23.825 | −21.996 | 0.00 | 0.00 | −0.650 | 9.00 | −17.40 |
| ATOM | 2798 | HN | ARG | 499 | 18.505 | 24.851 | −21.939 | 0.00 | 0.00 | 0.439 | 0.00 | 0.00 |
| ATOM | 2799 | CA | ARG | 499 | 18.224 | 23.049 | −20.817 | 0.00 | 0.00 | 0.158 | 9.40 | 4.00 |
| ATOM | 2800 | HA | ARG | 499 | 18.703 | 22.086 | −20.995 | 0.00 | 0.00 | 0.053 | 0.00 | 0.00 |
| ATOM | 2801 | C | ARG | 499 | 16.750 | 22.929 | −20.655 | 0.00 | 0.00 | 0.396 | 9.82 | 4.00 |
| ATOM | 2802 | O | ARG | 499 | 15.986 | 23.629 | −21.317 | 0.00 | 0.00 | −0.396 | 8.17 | −17.40 |
| ATOM | 2803 | CB | ARG | 499 | 18.860 | 23.660 | −19.565 | 0.00 | 0.00 | −0.106 | 12.77 | 4.00 |
| ATOM | 2804 | HB1 | ARG | 499 | 18.546 | 23.123 | −18.670 | 0.00 | 0.00 | 0.053 | 0.00 | 0.00 |
| ATOM | 2805 | HB2 | ARG | 499 | 18.569 | 24.704 | −19.453 | 0.00 | 0.00 | 0.053 | 0.00 | 0.00 |
| ATOM | 2806 | CG | ARG | 499 | 20.386 | 23.598 | −19.646 | 0.00 | 0.00 | −0.106 | 12.77 | 4.00 |
| ATOM | 2807 | HG1 | ARG | 499 | 20.702 | 24.106 | −20.557 | 0.00 | 0.00 | 0.053 | 0.00 | 0.00 |
| ATOM | 2808 | HG2 | ARG | 499 | 20.686 | 22.550 | −19.668 | 0.00 | 0.00 | 0.053 | 0.00 | 0.00 |
| ATOM | 2809 | CD | ARG | 499 | 21.098 | 24.261 | −18.472 | 0.00 | 0.00 | 0.374 | 12.77 | 4.00 |
| ATOM | 2810 | HD1 | ARG | 499 | 20.882 | 25.328 | −18.532 | 0.00 | 0.00 | 0.053 | 0.00 | 0.00 |
| ATOM | 2811 | HD2 | ARG | 499 | 22.162 | 24.054 | −18.589 | 0.00 | 0.00 | 0.053 | 0.00 | 0.00 |
| ATOM | 2812 | NE | ARG | 499 | 20.547 | 23.652 | −17.233 | 0.00 | 0.00 | −0.819 | 9.00 | −24.67 |
| ATOM | 2813 | HE | ARG | 499 | 19.595 | 23.259 | −17.229 | 0.00 | 0.00 | 0.407 | 0.00 | 0.00 |
| ATOM | 2814 | CZ | ARG | 499 | 21.307 | 23.616 | −16.102 | 0.00 | 0.00 | 0.796 | 6.95 | 4.00 |
| ATOM | 2815 | NH1 | ARG | 499 | 22.583 | 24.100 | −16.120 | 0.00 | 0.00 | −0.746 | 9.00 | −24.67 |
| ATOM | 2816 | HH11 | ARG | 499 | 23.156 | 24.072 | −15.265 | 0.00 | 0.00 | 0.407 | 0.00 | 0.00 |
| ATOM | 2817 | HH12 | ARG | 499 | 22.973 | 24.493 | −16.989 | 0.00 | 0.00 | 0.407 | 0.00 | 0.00 |
| ATOM | 2818 | NH2 | ARG | 499 | 20.790 | 23.087 | −14.954 | 0.00 | 0.00 | −0.746 | 9.00 | −24.67 |
| ATOM | 2819 | HH21 | ARG | 499 | 21.361 | 23.058 | −14.097 | 0.00 | 0.00 | 0.407 | 0.00 | 0.00 |
| ATOM | 2820 | HH22 | ARG | 499 | 19.829 | 22.717 | −14.945 | 0.00 | 0.00 | 0.407 | 0.00 | 0.00 |
| ATOM | 2821 | N | ASP | 500 | 16.301 | 22.034 | −19.761 | 0.00 | 0.00 | −0.650 | 9.00 | −17.40 |
| ATOM | 2822 | HN | ASP | 500 | 16.942 | 21.556 | −19.112 | 0.00 | 0.00 | 0.439 | 0.00 | 0.00 |
| ATOM | 2823 | CA | ASP | 500 | 14.898 | 21.774 | −19.749 | 0.00 | 0.00 | 0.158 | 9.40 | 4.00 |
| ATOM | 2824 | HA | ASP | 500 | 14.399 | 22.203 | −20.618 | 0.00 | 0.00 | 0.053 | 0.00 | 0.00 |
| ATOM | 2825 | C | ASP | 500 | 14.243 | 22.342 | −18.548 | 0.00 | 0.00 | 0.396 | 9.82 | 4.00 |
| ATOM | 2826 | O | ASP | 500 | 14.844 | 22.532 | −17.496 | 0.00 | 0.00 | −0.396 | 8.17 | −17.40 |
| ATOM | 2827 | CB | ASP | 500 | 14.566 | 20.276 | −19.673 | 0.00 | 0.00 | −0.336 | 12.77 | 4.00 |
| ATOM | 2828 | HB1 | ASP | 500 | 13.486 | 20.176 | −19.564 | 0.00 | 0.00 | 0.053 | 0.00 | 0.00 |
| ATOM | 2829 | HB2 | ASP | 500 | 15.085 | 19.860 | −18.809 | 0.00 | 0.00 | 0.053 | 0.00 | 0.00 |
| ATOM | 2830 | CG | ASP | 500 | 15.043 | 19.627 | −20.954 | 0.00 | 0.00 | 0.297 | 9.82 | 4.00 |
| ATOM | 2831 | OD1 | ASP | 500 | 14.814 | 20.241 | −22.027 | 0.00 | 0.00 | −0.534 | 8.17 | −18.95 |
| ATOM | 2832 | OD2 | ASP | 500 | 15.634 | 18.516 | −20.887 | 0.00 | 0.00 | −0.534 | 8.17 | −18.95 |
| ATOM | 2833 | N | LEU | 501 | 12.960 | 22.672 | −18.730 | 0.00 | 0.00 | −0.650 | 9.00 | −17.40 |
| ATOM | 2834 | HN | LEU | 501 | 12.592 | 22.750 | −19.689 | 0.00 | 0.00 | 0.439 | 0.00 | 0.00 |
| ATOM | 2835 | CA | LEU | 501 | 12.092 | 22.917 | −17.633 | 0.00 | 0.00 | 0.158 | 9.40 | 4.00 |
| ATOM | 2836 | HA | LEU | 501 | 12.630 | 23.072 | −16.698 | 0.00 | 0.00 | 0.053 | 0.00 | 0.00 |
| ATOM | 2837 | C | LEU | 501 | 11.346 | 21.626 | −17.708 | 0.00 | 0.00 | 0.396 | 9.82 | 4.00 |
| ATOM | 2838 | O | LEU | 501 | 10.991 | 21.207 | −18.808 | 0.00 | 0.00 | −0.396 | 8.17 | −17.40 |
| ATOM | 2839 | CB | LEU | 501 | 11.121 | 24.092 | −17.876 | 0.00 | 0.00 | −0.106 | 12.77 | 4.00 |
| ATOM | 2840 | HB1 | LEU | 501 | 10.396 | 24.231 | −17.074 | 0.00 | 0.00 | 0.053 | 0.00 | 0.00 |
| ATOM | 2841 | HB2 | LEU | 501 | 10.525 | 23.984 | −18.782 | 0.00 | 0.00 | 0.053 | 0.00 | 0.00 |
| ATOM | 2842 | CG | LEU | 501 | 11.830 | 25.460 | −18.021 | 0.00 | 0.00 | −0.053 | 9.40 | 4.00 |
| ATOM | 2843 | HG | LEU | 501 | 12.447 | 25.683 | −17.151 | 0.00 | 0.00 | 0.053 | 0.00 | 0.00 |
| ATOM | 2844 | CD1 | LEU | 501 | 12.746 | 25.490 | −19.256 | 0.00 | 0.00 | −0.159 | 16.15 | 4.00 |
| ATOM | 2845 | HD11 | LEU | 501 | 13.228 | 26.465 | −19.328 | 0.00 | 0.00 | 0.053 | 0.00 | 0.00 |
| ATOM | 2846 | HD12 | LEU | 501 | 13.507 | 24.715 | −19.164 | 0.00 | 0.00 | 0.053 | 0.00 | 0.00 |
| ATOM | 2847 | HD13 | LEU | 501 | 12.153 | 25.312 | −20.153 | 0.00 | 0.00 | 0.053 | 0.00 | 0.00 |
| ATOM | 2848 | CD2 | LEU | 501 | 10.830 | 26.630 | −18.004 | 0.00 | 0.00 | −0.159 | 16.15 | 4.00 |
| ATOM | 2849 | HD21 | LEU | 501 | 11.370 | 27.571 | −18.108 | 0.00 | 0.00 | 0.053 | 0.00 | 0.00 |
| ATOM | 2850 | HD22 | LEU | 501 | 10.128 | 26.521 | −18.831 | 0.00 | 0.00 | 0.053 | 0.00 | 0.00 |
| ATOM | 2851 | HD23 | LEU | 501 | 10.283 | 26.628 | −17.061 | 0.00 | 0.00 | 0.053 | 0.00 | 0.00 |
| ATOM | 2852 | N | GLN | 502 | 11.078 | 20.914 | −16.599 | 0.00 | 0.00 | −0.650 | 9.00 | −17.40 |
| ATOM | 2853 | HN | GLN | 502 | 11.257 | 21.252 | −15.643 | 0.00 | 0.00 | 0.439 | 0.00 | 0.00 |
| ATOM | 2854 | CA | GLN | 502 | 10.512 | 19.626 | −16.901 | 0.00 | 0.00 | 0.158 | 9.40 | 4.00 |
| ATOM | 2855 | HA | GLN | 502 | 10.071 | 19.567 | −17.896 | 0.00 | 0.00 | 0.053 | 0.00 | 0.00 |
| ATOM | 2856 | C | GLN | 502 | 9.417 | 19.220 | −15.964 | 0.00 | 0.00 | 0.396 | 9.82 | 4.00 |
| ATOM | 2857 | O | GLN | 502 | 8.727 | 20.060 | −15.400 | 0.00 | 0.00 | −0.396 | 8.17 | −17.40 |
| ATOM | 2858 | CB | GLN | 502 | 11.573 | 18.516 | −17.007 | 0.00 | 0.00 | −0.106 | 12.77 | 4.00 |
| ATOM | 2859 | HB1 | GLN | 502 | 12.268 | 18.791 | −17.801 | 0.00 | 0.00 | 0.053 | 0.00 | 0.00 |
| ATOM | 2860 | HB2 | GLN | 502 | 11.060 | 17.583 | −17.242 | 0.00 | 0.00 | 0.053 | 0.00 | 0.00 |
| ATOM | 2861 | CG | GLN | 502 | 12.403 | 18.268 | −15.749 | 0.00 | 0.00 | −0.106 | 12.77 | 4.00 |
| ATOM | 2862 | HG1 | GLN | 502 | 11.705 | 17.897 | −14.998 | 0.00 | 0.00 | 0.053 | 0.00 | 0.00 |
| ATOM | 2863 | HG2 | GLN | 502 | 12.837 | 19.232 | −15.482 | 0.00 | 0.00 | 0.053 | 0.00 | 0.00 |
| ATOM | 2864 | CD | GLN | 502 | 13.453 | 17.236 | −16.138 | 0.00 | 0.00 | 0.396 | 9.82 | 4.00 |
| ATOM | 2865 | OE1 | GLN | 502 | 13.602 | 16.914 | −17.316 | 0.00 | 0.00 | −0.396 | 8.17 | −17.40 |
| ATOM | 2866 | NE2 | GLN | 502 | 14.198 | 16.703 | −15.133 | 0.00 | 0.00 | −0.879 | 13.25 | −17.40 |
| ATOM | 2867 | HE21 | GLN | 502 | 14.039 | 17.002 | −14.160 | 0.00 | 0.00 | 0.439 | 0.00 | 0.00 |
| ATOM | 2868 | HE22 | GLN | 502 | 14.921 | 16.000 | −15.345 | 0.00 | 0.00 | 0.439 | 0.00 | 0.00 |
| ATOM | 2869 | N | PHE | 503 | 9.219 | 17.895 | −15.798 | 0.00 | 0.00 | −0.650 | 9.00 | −17.40 |
| ATOM | 2870 | HN | PHE | 503 | 9.899 | 17.244 | −16.216 | 0.00 | 0.00 | 0.439 | 0.00 | 0.00 |
| ATOM | 2871 | CA | PHE | 503 | 8.108 | 17.344 | −15.068 | 0.00 | 0.00 | 0.158 | 9.40 | 4.00 |
| ATOM | 2872 | HA | PHE | 503 | 7.223 | 17.392 | −15.703 | 0.00 | 0.00 | 0.053 | 0.00 | 0.00 |
| ATOM | 2873 | C | PHE | 503 | 7.794 | 18.060 | −13.800 | 0.00 | 0.00 | 0.396 | 9.82 | 4.00 |
| ATOM | 2874 | O | PHE | 503 | 8.639 | 18.232 | −12.923 | 0.00 | 0.00 | −0.396 | 8.17 | −17.40 |
| ATOM | 2875 | CB | PHE | 503 | 8.176 | 15.820 | −14.913 | 0.00 | 0.00 | −0.106 | 12.77 | 4.00 |
| ATOM | 2876 | HB1 | PHE | 503 | 7.549 | 15.446 | −14.103 | 0.00 | 0.00 | 0.053 | 0.00 | 0.00 |
| ATOM | 2877 | HB2 | PHE | 503 | 9.182 | 15.459 | −14.700 | 0.00 | 0.00 | 0.053 | 0.00 | 0.00 |
| ATOM | 2878 | CG | PHE | 503 | 7.683 | 15.515 | −16.268 | 0.00 | 0.00 | 0.000 | 7.26 | 0.60 |
| ATOM | 2879 | CD1 | PHE | 503 | 6.327 | 15.447 | −16.464 | 0.00 | 0.00 | −0.127 | 10.80 | 0.60 |
| ATOM | 2880 | HD1 | PHE | 503 | 5.649 | 15.586 | −15.622 | 0.00 | 0.00 | 0.127 | 0.00 | 0.00 |
| ATOM | 2881 | CD2 | PHE | 503 | 8.553 | 15.371 | −17.320 | 0.00 | 0.00 | −0.127 | 10.80 | 0.60 |
| ATOM | 2882 | HD2 | PHE | 503 | 9.628 | 15.449 | −17.160 | 0.00 | 0.00 | 0.127 | 0.00 | 0.00 |
| ATOM | 2883 | CE1 | PHE | 503 | 5.822 | 15.206 | −17.710 | 0.00 | 0.00 | −0.127 | 10.80 | 0.60 |
| ATOM | 2884 | HE1 | PHE | 503 | 4.747 | 15.138 | −17.874 | 0.00 | 0.00 | 0.127 | 0.00 | 0.00 |
| ATOM | 2885 | CE2 | PHE | 503 | 8.057 | 15.128 | −18.572 | 0.00 | 0.00 | −0.127 | 10.80 | 0.60 |
| ATOM | 2886 | HE2 | PHE | 503 | 8.731 | 14.997 | −19.418 | 0.00 | 0.00 | 0.127 | 0.00 | 0.00 |
| ATOM | 2887 | CZ | PHE | 503 | 6.701 | 15.052 | −18.745 | 0.00 | 0.00 | −0.127 | 10.80 | 0.60 |
| ATOM | 2888 | HZ | PHE | 503 | 6.308 | 14.862 | −19.744 | 0.00 | 0.00 | 0.127 | 0.00 | 0.00 |
| ATOM | 2889 | N | LEU | 504 | 6.523 | 18.529 | −13.733 | 0.00 | 0.00 | −0.650 | 9.00 | −17.40 |
| ATOM | 2890 | HN | LEU | 504 | 5.882 | 18.298 | −14.505 | 0.00 | 0.00 | 0.439 | 0.00 | 0.00 |
| ATOM | 2891 | CA | LEU | 504 | 6.018 | 19.325 | −12.655 | 0.00 | 0.00 | 0.158 | 9.40 | 4.00 |
| ATOM | 2892 | HA | LEU | 504 | 6.901 | 19.729 | −12.159 | 0.00 | 0.00 | 0.053 | 0.00 | 0.00 |
| ATOM | 2893 | C | LEU | 504 | 5.240 | 18.380 | −11.800 | 0.00 | 0.00 | 0.396 | 9.82 | 4.00 |
| ATOM | 2894 | O | LEU | 504 | 4.881 | 17.289 | −12.234 | 0.00 | 0.00 | −0.396 | 8.17 | −17.40 |
| ATOM | 2895 | CB | LEU | 504 | 5.083 | 20.450 | −13.180 | 0.00 | 0.00 | −0.106 | 12.77 | 4.00 |
| ATOM | 2896 | HB1 | LEU | 504 | 4.104 | 19.995 | −13.333 | 0.00 | 0.00 | 0.053 | 0.00 | 0.00 |
| ATOM | 2897 | HB2 | LEU | 504 | 5.520 | 20.809 | −14.112 | 0.00 | 0.00 | 0.053 | 0.00 | 0.00 |
| ATOM | 2898 | CG | LEU | 504 | 4.813 | 21.724 | −12.339 | 0.00 | 0.00 | −0.053 | 9.40 | 4.00 |
| ATOM | 2899 | HG | LEU | 504 | 4.061 | 22.323 | −12.854 | 0.00 | 0.00 | 0.053 | 0.00 | 0.00 |
| ATOM | 2900 | CD1 | LEU | 504 | 4.297 | 21.439 | −10.927 | 0.00 | 0.00 | −0.159 | 16.15 | 4.00 |
| ATOM | 2901 | HD11 | LEU | 504 | 4.133 | 22.380 | −10.403 | 0.00 | 0.00 | 0.053 | 0.00 | 0.00 |
| ATOM | 2902 | HD12 | LEU | 504 | 3.358 | 20.888 | −10.987 | 0.00 | 0.00 | 0.053 | 0.00 | 0.00 |
| ATOM | 2903 | HD13 | LEU | 504 | 5.032 | 20.845 | −10.384 | 0.00 | 0.00 | 0.053 | 0.00 | 0.00 |
| ATOM | 2904 | CD2 | LEU | 504 | 5.981 | 22.719 | −12.404 | 0.00 | 0.00 | −0.159 | 16.15 | 4.00 |
| ATOM | 2905 | HD21 | LEU | 504 | 5.746 | 23.595 | −11.800 | 0.00 | 0.00 | 0.053 | 0.00 | 0.00 |
| ATOM | 2906 | HD22 | LEU | 504 | 6.884 | 22.245 | −12.020 | 0.00 | 0.00 | 0.053 | 0.00 | 0.00 |
| ATOM | 2907 | HD23 | LEU | 504 | 6.142 | 23.024 | −13.438 | 0.00 | 0.00 | 0.053 | 0.00 | 0.00 |
| ATOM | 2908 | N | LEU | 505 | 5.060 | 18.764 | −10.527 | 0.00 | 0.00 | −0.650 | 9.00 | −17.40 |
| ATOM | 2909 | HN | LEU | 505 | 5.520 | 19.643 | −10.249 | 0.00 | 0.00 | 0.439 | 0.00 | 0.00 |
| ATOM | 2910 | CA | LEU | 505 | 4.307 | 18.100 | −9.511 | 0.00 | 0.00 | 0.158 | 9.40 | 4.00 |
| ATOM | 2911 | HA | LEU | 505 | 3.437 | 18.722 | −9.298 | 0.00 | 0.00 | 0.053 | 0.00 | 0.00 |
| ATOM | 2912 | C | LEU | 505 | 5.195 | 17.969 | −8.323 | 0.00 | 0.00 | 0.396 | 9.82 | 4.00 |
| ATOM | 2913 | O | LEU | 505 | 6.232 | 18.626 | −8.248 | 0.00 | 0.00 | −0.396 | 8.17 | −17.40 |
| ATOM | 2914 | CB | LEU | 505 | 3.578 | 16.788 | −9.872 | 0.00 | 0.00 | −0.106 | 12.77 | 4.00 |
| ATOM | 2915 | HB1 | LEU | 505 | 3.403 | 16.162 | −8.997 | 0.00 | 0.00 | 0.053 | 0.00 | 0.00 |
| ATOM | 2916 | HB2 | LEU | 505 | 4.146 | 16.185 | −10.580 | 0.00 | 0.00 | 0.053 | 0.00 | 0.00 |
| ATOM | 2917 | CG | LEU | 505 | 2.192 | 17.049 | −10.519 | 0.00 | 0.00 | −0.053 | 9.40 | 4.00 |
| ATOM | 2918 | HG | LEU | 505 | 1.589 | 16.144 | −10.447 | 0.00 | 0.00 | 0.053 | 0.00 | 0.00 |
| ATOM | 2919 | CD1 | LEU | 505 | 1.500 | 18.196 | −9.776 | 0.00 | 0.00 | −0.159 | 16.15 | 4.00 |
| ATOM | 2920 | HD11 | LEU | 505 | 0.525 | 18.385 | −10.225 | 0.00 | 0.00 | 0.053 | 0.00 | 0.00 |
| ATOM | 2921 | HD12 | LEU | 505 | 1.370 | 17.925 | −8.728 | 0.00 | 0.00 | 0.053 | 0.00 | 0.00 |
| ATOM | 2922 | HD13 | LEU | 505 | 2.112 | 19.096 | −9.845 | 0.00 | 0.00 | 0.053 | 0.00 | 0.00 |
| ATOM | 2923 | CD2 | LEU | 505 | 2.173 | 17.224 | −12.045 | 0.00 | 0.00 | −0.159 | 16.15 | 4.00 |
| ATOM | 2924 | HD21 | LEU | 505 | 1.150 | 17.400 | −12.378 | 0.00 | 0.00 | 0.053 | 0.00 | 0.00 |
| ATOM | 2925 | HD22 | LEU | 505 | 2.796 | 18.074 | −12.322 | 0.00 | 0.00 | 0.053 | 0.00 | 0.00 |
| ATOM | 2926 | HD23 | LEU | 505 | 2.559 | 16.322 | −12.520 | 0.00 | 0.00 | 0.053 | 0.00 | 0.00 |
| ATOM | 2927 | N | ARG | 506 | 4.823 | 17.117 | −7.358 | 0.00 | 0.00 | −0.650 | 9.00 | −17.40 |
| ATOM | 2928 | HN | ARG | 506 | 4.004 | 16.503 | −7.476 | 0.00 | 0.00 | 0.439 | 0.00 | 0.00 |
| ATOM | 2929 | CA | ARG | 506 | 5.600 | 17.089 | −6.160 | 0.00 | 0.00 | 0.158 | 9.40 | 4.00 |
| ATOM | 2930 | HA | ARG | 506 | 6.242 | 17.969 | −6.196 | 0.00 | 0.00 | 0.053 | 0.00 | 0.00 |
| ATOM | 2931 | C | ARG | 506 | 6.370 | 15.817 | −6.198 | 0.00 | 0.00 | 0.396 | 9.82 | 4.00 |
| ATOM | 2932 | O | ARG | 506 | 5.911 | 14.817 | −6.747 | 0.00 | 0.00 | −0.396 | 8.17 | −17.40 |
| ATOM | 2933 | CB | ARG | 506 | 4.692 | 17.125 | −4.917 | 0.00 | 0.00 | −0.106 | 12.77 | 4.00 |
| ATOM | 2934 | HB1 | ARG | 506 | 4.482 | 16.096 | −4.623 | 0.00 | 0.00 | 0.053 | 0.00 | 0.00 |
| ATOM | 2935 | HB2 | ARG | 506 | 3.774 | 17.648 | −5.186 | 0.00 | 0.00 | 0.053 | 0.00 | 0.00 |
| ATOM | 2936 | CG | ARG | 506 | 5.275 | 17.834 | −3.696 | 0.00 | 0.00 | −0.106 | 12.77 | 4.00 |
| ATOM | 2937 | HG1 | ARG | 506 | 5.954 | 18.628 | −4.005 | 0.00 | 0.00 | 0.053 | 0.00 | 0.00 |
| ATOM | 2938 | HG2 | ARG | 506 | 5.827 | 17.130 | −3.074 | 0.00 | 0.00 | 0.053 | 0.00 | 0.00 |
| ATOM | 2939 | CD | ARG | 506 | 4.157 | 18.453 | −2.852 | 0.00 | 0.00 | 0.374 | 12.77 | 4.00 |
| ATOM | 2940 | HD1 | ARG | 506 | 4.656 | 19.064 | −2.099 | 0.00 | 0.00 | 0.053 | 0.00 | 0.00 |
| ATOM | 2941 | HD2 | ARG | 506 | 3.610 | 17.614 | −2.421 | 0.00 | 0.00 | 0.053 | 0.00 | 0.00 |
| ATOM | 2942 | NE | ARG | 506 | 3.330 | 19.260 | −3.799 | 0.00 | 0.00 | −0.819 | 9.00 | −24.67 |
| ATOM | 2943 | HE | ARG | 506 | 2.608 | 18.793 | −4.365 | 0.00 | 0.00 | 0.407 | 0.00 | 0.00 |
| ATOM | 2944 | CZ | ARG | 506 | 3.516 | 20.606 | −3.926 | 0.00 | 0.00 | 0.796 | 6.95 | 4.00 |
| ATOM | 2945 | NH1 | ARG | 506 | 4.338 | 21.271 | −3.064 | 0.00 | 0.00 | −0.746 | 9.00 | −24.67 |
| ATOM | 2946 | HH11 | ARG | 506 | 4.475 | 22.287 | −3.164 | 0.00 | 0.00 | 0.407 | 0.00 | 0.00 |
| ATOM | 2947 | HH12 | ARG | 506 | 4.821 | 20.757 | −2.313 | 0.00 | 0.00 | 0.407 | 0.00 | 0.00 |
| ATOM | 2948 | NH2 | ARG | 506 | 2.879 | 21.292 | −4.921 | 0.00 | 0.00 | −0.746 | 9.00 | −24.67 |
| ATOM | 2949 | HH21 | ARG | 506 | 3.019 | 22.308 | −5.017 | 0.00 | 0.00 | 0.407 | 0.00 | 0.00 |
| ATOM | 2950 | HH22 | ARG | 506 | 2.259 | 20.793 | −5.575 | 0.00 | 0.00 | 0.407 | 0.00 | 0.00 |
| ATOM | 2951 | N | GLU | 507 | 7.593 | 15.825 | −5.649 | 0.00 | 0.00 | −0.650 | 9.00 | −17.40 |
| ATOM | 2952 | HN | GLU | 507 | 7.966 | 16.663 | −5.180 | 0.00 | 0.00 | 0.439 | 0.00 | 0.00 |
| ATOM | 2953 | CA | GLU | 507 | 8.355 | 14.623 | −5.742 | 0.00 | 0.00 | 0.158 | 9.40 | 4.00 |
| ATOM | 2954 | HA | GLU | 507 | 7.812 | 13.946 | −6.401 | 0.00 | 0.00 | 0.053 | 0.00 | 0.00 |
| ATOM | 2955 | C | GLU | 507 | 8.476 | 14.064 | −4.357 | 0.00 | 0.00 | 0.396 | 9.82 | 4.00 |
| ATOM | 2956 | O | GLU | 507 | 8.864 | 14.772 | −3.429 | 0.00 | 0.00 | −0.396 | 8.17 | −17.40 |
| ATOM | 2957 | CB | GLU | 507 | 9.705 | 14.898 | −6.417 | 0.00 | 0.00 | −0.106 | 12.77 | 4.00 |
| ATOM | 2958 | HB1 | GLU | 507 | 10.271 | 13.967 | −6.444 | 0.00 | 0.00 | 0.053 | 0.00 | 0.00 |
| ATOM | 2959 | HB2 | GLU | 507 | 10.234 | 15.651 | −5.832 | 0.00 | 0.00 | 0.053 | 0.00 | 0.00 |
| ATOM | 2960 | CG | GLU | 507 | 9.438 | 15.410 | −7.842 | 0.00 | 0.00 | −0.336 | 12.77 | 4.00 |
| ATOM | 2961 | HG1 | GLU | 507 | 8.765 | 16.268 | −7.835 | 0.00 | 0.00 | 0.053 | 0.00 | 0.00 |
| ATOM | 2962 | HG2 | GLU | 507 | 8.981 | 14.638 | −8.461 | 0.00 | 0.00 | 0.053 | 0.00 | 0.00 |
| ATOM | 2963 | CD | GLU | 507 | 10.725 | 15.842 | −8.523 | 0.00 | 0.00 | 0.297 | 9.82 | 4.00 |
| ATOM | 2964 | OE1 | GLU | 507 | 11.744 | 16.043 | −7.809 | 0.00 | 0.00 | −0.534 | 8.17 | −18.95 |
| ATOM | 2965 | OE2 | GLU | 507 | 10.708 | 15.964 | −9.777 | 0.00 | 0.00 | −0.534 | 8.17 | −18.95 |
| ATOM | 2966 | N | THR | 508 | 8.111 | 12.772 | −4.177 | 0.00 | 0.00 | −0.650 | 9.00 | −17.40 |
| ATOM | 2967 | HN | THR | 508 | 7.836 | 12.194 | −4.984 | 0.00 | 0.00 | 0.439 | 0.00 | 0.00 |
| ATOM | 2968 | CA | THR | 508 | 8.109 | 12.212 | −2.853 | 0.00 | 0.00 | 0.158 | 9.40 | 4.00 |
| ATOM | 2969 | HA | THR | 508 | 8.456 | 12.935 | −2.115 | 0.00 | 0.00 | 0.053 | 0.00 | 0.00 |
| ATOM | 2970 | C | THR | 508 | 8.998 | 11.023 | −2.766 | 0.00 | 0.00 | 0.396 | 9.82 | 4.00 |
| ATOM | 2971 | O | THR | 508 | 9.220 | 10.289 | −3.729 | 0.00 | 0.00 | −0.396 | 8.17 | −17.40 |
| ATOM | 2972 | CB | THR | 508 | 6.776 | 11.733 | −2.357 | 0.00 | 0.00 | 0.060 | 9.40 | 4.00 |
| ATOM | 2973 | HB | THR | 508 | 6.051 | 12.533 | −2.507 | 0.00 | 0.00 | 0.053 | 0.00 | 0.00 |
| ATOM | 2974 | OG1 | THR | 508 | 6.865 | 11.385 | −0.982 | 0.00 | 0.00 | −0.537 | 11.04 | −17.40 |
| ATOM | 2975 | HG1 | THR | 508 | 6.649 | 12.210 | −0.404 | 0.00 | 0.00 | 0.424 | 0.00 | 0.00 |
| ATOM | 2976 | CG2 | THR | 508 | 6.388 | 10.489 | −3.162 | 0.00 | 0.00 | −0.159 | 16.15 | 4.00 |
| ATOM | 2977 | HG21 | THR | 508 | 5.421 | 10.121 | −2.820 | 0.00 | 0.00 | 0.053 | 0.00 | 0.00 |
| ATOM | 2978 | HG22 | THR | 508 | 6.325 | 10.745 | −4.220 | 0.00 | 0.00 | 0.053 | 0.00 | 0.00 |
| ATOM | 2979 | HG23 | THR | 508 | 7.142 | 9.714 | −3.020 | 0.00 | 0.00 | 0.053 | 0.00 | 0.00 |
| ATOM | 2980 | N | LYS | 509 | 9.520 | 10.837 | −1.545 | 0.00 | 0.00 | −0.650 | 9.00 | −17.40 |
| ATOM | 2981 | HN | LYS | 509 | 9.235 | 11.506 | −0.815 | 0.00 | 0.00 | 0.439 | 0.00 | 0.00 |
| ATOM | 2982 | CA | LYS | 509 | 10.429 | 9.810 | −1.150 | 0.00 | 0.00 | 0.158 | 9.40 | 4.00 |
| ATOM | 2983 | HA | LYS | 509 | 11.242 | 9.721 | −1.870 | 0.00 | 0.00 | 0.053 | 0.00 | 0.00 |
| ATOM | 2984 | C | LYS | 509 | 9.723 | 8.483 | −1.065 | 0.00 | 0.00 | 0.396 | 9.82 | 4.00 |
| ATOM | 2985 | O | LYS | 509 | 10.332 | 7.443 | −1.306 | 0.00 | 0.00 | −0.396 | 8.17 | −17.40 |
| ATOM | 2986 | CB | LYS | 509 | 11.028 | 10.126 | 0.230 | 0.00 | 0.00 | −0.106 | 12.77 | 4.00 |
| ATOM | 2987 | HB1 | LYS | 509 | 11.697 | 9.337 | 0.573 | 0.00 | 0.00 | 0.053 | 0.00 | 0.00 |
| ATOM | 2988 | HB2 | LYS | 509 | 10.256 | 10.242 | 0.991 | 0.00 | 0.00 | 0.053 | 0.00 | 0.00 |
| ATOM | 2989 | CG | LYS | 509 | 11.840 | 11.425 | 0.216 | 0.00 | 0.00 | −0.106 | 12.77 | 4.00 |
| ATOM | 2990 | HG1 | LYS | 509 | 11.293 | 12.162 | −0.372 | 0.00 | 0.00 | 0.053 | 0.00 | 0.00 |
| ATOM | 2991 | HG2 | LYS | 509 | 12.810 | 11.215 | −0.235 | 0.00 | 0.00 | 0.053 | 0.00 | 0.00 |
| ATOM | 2992 | CD | LYS | 509 | 12.095 | 12.034 | 1.596 | 0.00 | 0.00 | −0.106 | 12.77 | 4.00 |
| ATOM | 2993 | HD1 | LYS | 509 | 12.619 | 11.356 | 2.270 | 0.00 | 0.00 | 0.053 | 0.00 | 0.00 |
| ATOM | 2994 | HD2 | LYS | 509 | 11.178 | 12.315 | 2.113 | 0.00 | 0.00 | 0.053 | 0.00 | 0.00 |
| ATOM | 2995 | CE | LYS | 509 | 12.947 | 13.305 | 1.540 | 0.00 | 0.00 | 0.099 | 12.77 | 4.00 |
| ATOM | 2996 | HE1 | LYS | 509 | 12.614 | 13.946 | 0.724 | 0.00 | 0.00 | 0.053 | 0.00 | 0.00 |
| ATOM | 2997 | HE2 | LYS | 509 | 13.994 | 13.050 | 1.379 | 0.00 | 0.00 | 0.053 | 0.00 | 0.00 |
| ATOM | 2998 | NZ | LYS | 509 | 12.831 | 14.055 | 2.810 | 0.00 | 0.00 | −0.045 | 13.25 | −39.20 |
| ATOM | 2999 | HZ1 | LYS | 509 | 13.408 | 14.907 | 2.760 | 0.00 | 0.00 | 0.280 | 0.00 | 0.00 |
| ATOM | 3000 | HZ2 | LYS | 509 | 11.847 | 14.316 | 2.967 | 0.00 | 0.00 | 0.280 | 0.00 | 0.00 |
| ATOM | 3001 | HZ3 | LYS | 509 | 13.157 | 13.465 | 3.589 | 0.00 | 0.00 | 0.280 | 0.00 | 0.00 |
| ATOM | 3002 | N | LEU | 510 | 8.419 | 8.477 | −0.715 | 0.00 | 0.00 | −0.650 | 9.00 | −17.40 |
| ATOM | 3003 | HN | LEU | 510 | 7.911 | 9.368 | −0.624 | 0.00 | 0.00 | 0.439 | 0.00 | 0.00 |
| ATOM | 3004 | CA | LEU | 510 | 7.732 | 7.235 | −0.467 | 0.00 | 0.00 | 0.158 | 9.40 | 4.00 |
| ATOM | 3005 | HA | LEU | 510 | 8.409 | 6.507 | −0.020 | 0.00 | 0.00 | 0.053 | 0.00 | 0.00 |
| ATOM | 3006 | C | LEU | 510 | 7.197 | 6.660 | −1.756 | 0.00 | 0.00 | 0.396 | 9.82 | 4.00 |
| ATOM | 3007 | O | LEU | 510 | 6.411 | 7.297 | −2.454 | 0.00 | 0.00 | −0.396 | 8.17 | −17.40 |
| ATOM | 3008 | CB | LEU | 510 | 6.552 | 7.418 | 0.512 | 0.00 | 0.00 | −0.106 | 12.77 | 4.00 |
| ATOM | 3009 | HB1 | LEU | 510 | 5.753 | 7.935 | −0.019 | 0.00 | 0.00 | 0.053 | 0.00 | 0.00 |
| ATOM | 3010 | HB2 | LEU | 510 | 6.906 | 8.009 | 1.357 | 0.00 | 0.00 | 0.053 | 0.00 | 0.00 |
| ATOM | 3011 | CG | LEU | 510 | 5.945 | 6.120 | 1.090 | 0.00 | 0.00 | −0.053 | 9.40 | 4.00 |
| ATOM | 3012 | HG | LEU | 510 | 5.157 | 6.315 | 1.818 | 0.00 | 0.00 | 0.053 | 0.00 | 0.00 |
| ATOM | 3013 | CD1 | LEU | 510 | 5.318 | 5.230 | 0.006 | 0.00 | 0.00 | −0.159 | 16.15 | 4.00 |
| ATOM | 3014 | HD11 | LEU | 510 | 4.906 | 4.332 | 0.466 | 0.00 | 0.00 | 0.053 | 0.00 | 0.00 |
| ATOM | 3015 | HD12 | LEU | 510 | 4.521 | 5.777 | −0.498 | 0.00 | 0.00 | 0.053 | 0.00 | 0.00 |
| ATOM | 3016 | HD13 | LEU | 510 | 6.081 | 4.949 | −0.720 | 0.00 | 0.00 | 0.053 | 0.00 | 0.00 |
| ATOM | 3017 | CD2 | LEU | 510 | 6.961 | 5.380 | 1.976 | 0.00 | 0.00 | −0.159 | 16.15 | 4.00 |
| ATOM | 3018 | HD21 | LEU | 510 | 6.507 | 4.471 | 2.369 | 0.00 | 0.00 | 0.053 | 0.00 | 0.00 |
| ATOM | 3019 | HD22 | LEU | 510 | 7.839 | 5.120 | 1.384 | 0.00 | 0.00 | 0.053 | 0.00 | 0.00 |
| ATOM | 3020 | HD23 | LEU | 510 | 7.259 | 6.024 | 2.804 | 0.00 | 0.00 | 0.053 | 0.00 | 0.00 |
| ATOM | 3021 | N | PHE | 511 | 7.606 | 5.409 | −2.075 | 0.00 | 0.00 | −0.650 | 9.00 | −17.40 |
| ATOM | 3022 | HN | PHE | 511 | 8.247 | 4.942 | −1.418 | 0.00 | 0.00 | 0.439 | 0.00 | 0.00 |
| ATOM | 3023 | CA | PHE | 511 | 7.222 | 4.671 | −3.257 | 0.00 | 0.00 | 0.158 | 9.40 | 4.00 |
| ATOM | 3024 | HA | PHE | 511 | 7.271 | 5.337 | −4.118 | 0.00 | 0.00 | 0.053 | 0.00 | 0.00 |
| ATOM | 3025 | C | PHE | 511 | 5.831 | 4.166 | −3.077 | 0.00 | 0.00 | 0.396 | 9.82 | 4.00 |
| ATOM | 3026 | O | PHE | 511 | 5.579 | 3.223 | −2.329 | 0.00 | 0.00 | −0.396 | 8.17 | 17.40 |
| ATOM | 3027 | CB | PHE | 511 | 8.145 | 3.462 | −3.509 | 0.00 | 0.00 | −0.106 | 12.77 | 4.00 |
| ATOM | 3028 | HB1 | PHE | 511 | 8.284 | 2.889 | −2.592 | 0.00 | 0.00 | 0.053 | 0.00 | 0.00 |
| ATOM | 3029 | HB2 | PHE | 511 | 9.123 | 3.793 | −3.858 | 0.00 | 0.00 | 0.053 | 0.00 | 0.00 |
| ATOM | 3030 | CG | PHE | 511 | 7.557 | 2.560 | −4.543 | 0.00 | 0.00 | 0.000 | 7.26 | 0.60 |
| ATOM | 3031 | CD1 | PHE | 511 | 7.651 | 2.846 | −5.886 | 0.00 | 0.00 | −0.127 | 10.80 | 0.60 |
| ATOM | 3032 | HD1 | PHE | 511 | 8.159 | 3.754 | −6.211 | 0.00 | 0.00 | 0.127 | 0.00 | 0.00 |
| ATOM | 3033 | CD2 | PHE | 511 | 6.924 | 1.399 | −4.157 | 0.00 | 0.00 | −0.127 | 10.80 | 0.60 |
| ATOM | 3034 | HD2 | PHE | 511 | 6.853 | 1.153 | −3.097 | 0.00 | 0.00 | 0.127 | 0.00 | 0.00 |
| ATOM | 3035 | CE1 | PHE | 511 | 7.109 | 1.996 | −6.822 | 0.00 | 0.00 | −0.127 | 10.80 | 0.60 |
| ATOM | 3036 | HE1 | PHE | 511 | 7.185 | 2.237 | −7.882 | 0.00 | 0.00 | 0.127 | 0.00 | 0.00 |
| ATOM | 3037 | CE2 | PHE | 511 | 6.381 | 0.546 | −5.088 | 0.00 | 0.00 | −0.127 | 10.80 | 0.60 |
| ATOM | 3038 | HE2 | PHE | 511 | 5.879 | −0.366 | −4.765 | 0.00 | 0.00 | 0.127 | 0.00 | 0.00 |
| ATOM | 3039 | CZ | PHE | 511 | 6.472 | 0.844 | −6.426 | 0.00 | 0.00 | −0.127 | 10.80 | 0.60 |
| ATOM | 3040 | HZ | PHE | 511 | 6.042 | 0.172 | −7.168 | 0.00 | 0.00 | 0.127 | 0.00 | 0.00 |
| ATOM | 3041 | N | VAL | 512 | 4.875 | 4.831 | −3.743 | 0.00 | 0.00 | −0.650 | 9.00 | −17.40 |
| ATOM | 3042 | HN | VAL | 512 | 5.139 | 5.606 | −4.368 | 0.00 | 0.00 | 0.439 | 0.00 | 0.00 |
| ATOM | 3043 | CA | VAL | 512 | 3.496 | 4.483 | −3.601 | 0.00 | 0.00 | 0.158 | 9.40 | 4.00 |
| ATOM | 3044 | HA | VAL | 512 | 3.327 | 4.209 | −2.560 | 0.00 | 0.00 | 0.053 | 0.00 | 0.00 |
| ATOM | 3045 | C | VAL | 512 | 3.006 | 3.329 | −4.445 | 0.00 | 0.00 | 0.396 | 9.82 | 4.00 |
| ATOM | 3046 | O | VAL | 512 | 2.026 | 2.691 | −4.067 | 0.00 | 0.00 | −0.396 | 8.17 | −17.40 |
| ATOM | 3047 | CB | VAL | 512 | 2.607 | 5.684 | −3.513 | 0.00 | 0.00 | −0.053 | 9.40 | 4.00 |
| ATOM | 3048 | HB | VAL | 512 | 2.694 | 6.123 | −2.519 | 0.00 | 0.00 | 0.053 | 0.00 | 0.00 |
| ATOM | 3049 | CG1 | VAL | 512 | 3.035 | 6.706 | −4.568 | 0.00 | 0.00 | −0.159 | 16.15 | 4.00 |
| ATOM | 3050 | HG11 | VAL | 512 | 2.390 | 7.583 | −4.508 | 0.00 | 0.00 | 0.053 | 0.00 | 0.00 |
| ATOM | 3051 | HG12 | VAL | 512 | 4.068 | 7.003 | −4.389 | 0.00 | 0.00 | 0.053 | 0.00 | 0.00 |
| ATOM | 3052 | HG13 | VAL | 512 | 2.952 | 6.262 | −5.560 | 0.00 | 0.00 | 0.053 | 0.00 | 0.00 |
| ATOM | 3053 | CG2 | VAL | 512 | 1.145 | 5.221 | −3.468 | 0.00 | 0.00 | −0.159 | 16.15 | 4.00 |
| ATOM | 3054 | HG21 | VAL | 512 | 0.490 | 6.090 | −3.404 | 0.00 | 0.00 | 0.053 | 0.00 | 0.00 |
| ATOM | 3055 | HG22 | VAL | 512 | 0.914 | 4.658 | −4.372 | 0.00 | 0.00 | 0.053 | 0.00 | 0.00 |
| ATOM | 3056 | HG23 | VAL | 512 | 0.991 | 4.586 | −2.596 | 0.00 | 0.00 | 0.053 | 0.00 | 0.00 |
| ATOM | 3057 | N | GLY | 513 | 3.626 | 3.008 | −5.602 | 0.00 | 0.00 | −0.650 | 9.00 | −17.40 |
| ATOM | 3058 | HN | GLY | 513 | 4.414 | 3.575 | −5.946 | 0.00 | 0.00 | 0.439 | 0.00 | 0.00 |
| ATOM | 3059 | CA | GLY | 513 | 3.169 | 1.859 | −6.352 | 0.00 | 0.00 | 0.105 | 9.40 | 4.00 |
| ATOM | 3060 | HA1 | GLY | 513 | 3.005 | 1.025 | −5.670 | 0.00 | 0.00 | 0.053 | 0.00 | 0.00 |
| ATOM | 3061 | HA2 | GLY | 513 | 3.924 | 1.585 | −7.089 | 0.00 | 0.00 | 0.053 | 0.00 | 0.00 |
| ATOM | 3062 | C | GLY | 513 | 1.889 | 2.198 | −7.047 | 0.00 | 0.00 | 0.396 | 9.82 | 4.00 |
| ATOM | 3063 | O | GLY | 513 | 1.857 | 3.143 | −7.824 | 0.00 | 0.00 | −0.396 | 8.17 | −17.40 |
| ATOM | 3064 | N | LEU | 514 | 0.834 | 1.373 | −6.852 | 0.00 | 0.00 | −0.650 | 9.00 | −17.40 |
| ATOM | 3065 | HN | LEU | 514 | 1.001 | 0.516 | −6.305 | 0.00 | 0.00 | 0.439 | 0.00 | 0.00 |
| ATOM | 3066 | CA | LEU | 514 | −0.507 | 1.591 | −7.345 | 0.00 | 0.00 | 0.158 | 9.40 | 4.00 |
| ATOM | 3067 | HA | LEU | 514 | −1.087 | 0.672 | −7.429 | 0.00 | 0.00 | 0.053 | 0.00 | 0.00 |
| ATOM | 3068 | C | LEU | 514 | −0.539 | 2.213 | −8.708 | 0.00 | 0.00 | 0.396 | 9.82 | 4.00 |
| ATOM | 3069 | O | LEU | 514 | −1.078 | 3.304 | −8.886 | 0.00 | 0.00 | −0.396 | 8.17 | −17.40 |
| ATOM | 3070 | CB | LEU | 514 | −1.371 | 2.452 | −6.408 | 0.00 | 0.00 | −0.106 | 12.77 | 4.00 |
| ATOM | 3071 | HB1 | LEU | 514 | −2.333 | 2.726 | −6.840 | 0.00 | 0.00 | 0.053 | 0.00 | 0.00 |
| ATOM | 3072 | HB2 | LEU | 514 | −0.899 | 3.395 | −6.131 | 0.00 | 0.00 | 0.053 | 0.00 | 0.00 |
| ATOM | 3073 | CG | LEU | 514 | −1.704 | 1.756 | −5.077 | 0.00 | 0.00 | −0.053 | 9.40 | 4.00 |
| ATOM | 3074 | HG | LEU | 514 | −2.184 | 0.790 | −5.233 | 0.00 | 0.00 | 0.053 | 0.00 | 0.00 |
| ATOM | 3075 | CD1 | LEU | 514 | −0.446 | 1.500 | −4.235 | 0.00 | 0.00 | −0.159 | 16.15 | 4.00 |
| ATOM | 3076 | HD11 | LEU | 514 | −0.726 | 1.007 | −3.304 | 0.00 | 0.00 | 0.053 | 0.00 | 0.00 |
| ATOM | 3077 | HD12 | LEU | 514 | 0.241 | 0.862 | −4.791 | 0.00 | 0.00 | 0.053 | 0.00 | 0.00 |
| ATOM | 3078 | HD13 | LEU | 514 | 0.041 | 2.449 | −4.011 | 0.00 | 0.00 | 0.053 | 0.00 | 0.00 |
| ATOM | 3079 | CD2 | LEU | 514 | −2.785 | 2.526 | −4.307 | 0.00 | 0.00 | −0.159 | 16.15 | 4.00 |
| ATOM | 3080 | HD21 | LEU | 514 | −3.001 | 2.012 | −3.370 | 0.00 | 0.00 | 0.053 | 0.00 | 0.00 |
| ATOM | 3081 | HD22 | LEU | 514 | −2.431 | 3.535 | −4.094 | 0.00 | 0.00 | 0.053 | 0.00 | 0.00 |
| ATOM | 3082 | HD23 | LEU | 514 | −3.692 | 2.579 | −4.909 | 0.00 | 0.00 | 0.053 | 0.00 | 0.00 |
| ATOM | 3083 | N | GLY | 515 | 0.032 | 1.547 | −9.723 | 0.00 | 0.00 | −0.650 | 9.00 | −17.40 |
| ATOM | 3084 | HN | GLY | 515 | 0.487 | 0.636 | −9.571 | 0.00 | 0.00 | 0.439 | 0.00 | 0.00 |
| ATOM | 3085 | CA | GLY | 515 | −0.012 | 2.144 | −11.024 | 0.00 | 0.00 | 0.105 | 9.40 | 4.00 |
| ATOM | 3086 | HA1 | GLY | 515 | −0.894 | 2.784 | −11.064 | 0.00 | 0.00 | 0.053 | 0.00 | 0.00 |
| ATOM | 3087 | HA2 | GLY | 515 | −0.076 | 1.338 | −11.755 | 0.00 | 0.00 | 0.053 | 0.00 | 0.00 |
| ATOM | 3088 | C | GLY | 515 | 1.235 | 2.933 | −11.199 | 0.00 | 0.00 | 0.396 | 9.82 | 4.00 |
| ATOM | 3089 | O | GLY | 515 | 1.255 | 4.143 | −10.970 | 0.00 | 0.00 | −0.396 | 8.17 | −17.40 |
| ATOM | 3090 | N | PHE | 516 | 2.322 | 2.257 | −11.611 | 0.00 | 0.00 | −0.650 | 9.00 | −17.40 |
| ATOM | 3091 | HN | PHE | 516 | 2.274 | 1.246 | −11.802 | 0.00 | 0.00 | 0.439 | 0.00 | 0.00 |
| ATOM | 3092 | CA | PHE | 516 | 3.547 | 2.978 | −11.777 | 0.00 | 0.00 | 0.158 | 9.40 | 4.00 |
| ATOM | 3093 | HA | PHE | 516 | 3.323 | 4.003 | −12.074 | 0.00 | 0.00 | 0.053 | 0.00 | 0.00 |
| ATOM | 3094 | C | PHE | 516 | 4.370 | 2.314 | −12.836 | 0.00 | 0.00 | 0.396 | 9.82 | 4.00 |
| ATOM | 3095 | O | PHE | 516 | 4.431 | 1.087 | −12.931 | 0.00 | 0.00 | −0.396 | 8.17 | −17.40 |
| ATOM | 3096 | CB | PHE | 516 | 4.367 | 3.041 | −10.479 | 0.00 | 0.00 | −0.106 | 12.77 | 4.00 |
| ATOM | 3097 | HB1 | PHE | 516 | 3.774 | 3.636 | −9.785 | 0.00 | 0.00 | 0.053 | 0.00 | 0.00 |
| ATOM | 3098 | HB2 | PHE | 516 | 5.310 | 3.517 | −10.749 | 0.00 | 0.00 | 0.053 | 0.00 | 0.00 |
| ATOM | 3099 | CG | PHE | 516 | 4.533 | 1.629 | −10.040 | 0.00 | 0.00 | 0.000 | 7.26 | 0.60 |
| ATOM | 3100 | CD1 | PHE | 516 | 3.549 | 1.016 | −9.297 | 0.00 | 0.00 | −0.127 | 10.80 | 0.60 |
| ATOM | 3101 | HD1 | PHE | 516 | 2.650 | 1.571 | −9.029 | 0.00 | 0.00 | 0.127 | 0.00 | 0.00 |
| ATOM | 3102 | CD2 | PHE | 516 | 5.659 | 0.918 | −10.376 | 0.00 | 0.00 | −0.127 | 10.80 | 0.60 |
| ATOM | 3103 | HD2 | PHE | 516 | 6.442 | 1.392 | −10.968 | 0.00 | 0.00 | 0.127 | 0.00 | 0.00 |
| ATOM | 3104 | CE1 | PHE | 516 | 3.689 | −0.289 | −8.890 | 0.00 | 0.00 | −0.127 | 10.80 | 0.60 |
| ATOM | 3105 | HE1 | PHE | 516 | 2.904 | −0.765 | −8.302 | 0.00 | 0.00 | 0.127 | 0.00 | 0.00 |
| ATOM | 3106 | CE2 | PHE | 516 | 5.804 | −0.387 | −9.971 | 0.00 | 0.00 | −0.127 | 10.80 | 0.60 |
| ATOM | 3107 | HE2 | PHE | 516 | 6.702 | −0.943 | −10.241 | 0.00 | 0.00 | 0.127 | 0.00 | 0.00 |
| ATOM | 3108 | CZ | PHE | 516 | 4.820 | −0.993 | −9.226 | 0.00 | 0.00 | −0.127 | 10.80 | 60 |
| ATOM | 3109 | HZ | PHE | 516 | 4.937 | −2.028 | −8.903 | 0.00 | 0.00 | 0.127 | 0.00 | 0.00 |
| ATOM | 3110 | N | PRO | 517 | 4.961 | 3.119 | −13.682 | 0.00 | 0.00 | −0.422 | 9.00 | −17.40 |
| ATOM | 3111 | CA | PRO | 517 | 5.839 | 2.555 | −14.670 | 0.00 | 0.00 | 0.158 | 9.40 | −4.00 |
| ATOM | 3112 | HA | PRO | 517 | 5.553 | 1.546 | −14.968 | 0.00 | 0.00 | 0.053 | 0.00 | 0.00 |
| ATOM | 3113 | CD | PRO | 517 | 4.205 | 4.231 | −14.235 | 0.00 | 0.00 | 0.105 | 12.77 | 4.00 |
| ATOM | 3114 | HD1 | PRO | 517 | 4.170 | 4.971 | −13.435 | 0.00 | 0.00 | 0.053 | 0.00 | 0.00 |
| ATOM | 3115 | HD2 | PRO | 517 | 3.231 | 3.810 | −14.485 | 0.00 | 0.00 | 0.053 | 0.00 | 0.00 |
| ATOM | 3116 | C | PRO | 517 | 7.254 | 2.453 | −14.203 | 0.00 | 0.00 | 0.396 | 9.82 | 4.00 |
| ATOM | 3117 | O | PRO | 517 | 7.660 | 3.250 | −13.355 | 0.00 | 0.00 | −0.396 | 8.17 | −17.40 |
| ATOM | 3118 | CB | PRO | 517 | 5.692 | 3.398 | −15.938 | 0.00 | 0.00 | −0.106 | 12.77 | 4.00 |
| ATOM | 3119 | HB1 | PRO | 517 | 5.075 | 2.787 | −16.597 | 0.00 | 0.00 | 0.053 | 0.00 | 0.00 |
| ATOM | 3120 | HB2 | PRO | 517 | 6.715 | 3.538 | −16.288 | 0.00 | 0.00 | 0.053 | 0.00 | 0.00 |
| ATOM | 3121 | CG | PRO | 517 | 5.012 | 4.686 | −15.460 | 0.00 | 0.00 | −0.106 | 12.77 | 4.00 |
| ATOM | 3122 | HG1 | PRO | 517 | 4.369 | 5.100 | −16.237 | 0.00 | 0.00 | 0.053 | 0.00 | 0.00 |
| ATOM | 3123 | HG2 | PRO | 517 | 5.750 | 5.444 | −15.198 | 0.00 | 0.00 | 0.053 | 0.00 | 0.00 |
| ATOM | 3124 | N | TYR | 518 | 8.024 | 1.485 | −14.751 | 0.00 | 0.00 | −0.650 | 9.00 | −17.40 |
| ATOM | 3125 | HN | TYR | 518 | 7.604 | 0.792 | −15.387 | 0.00 | 0.00 | 0.439 | 0.00 | 0.00 |
| ATOM | 3126 | CA | TYR | 518 | 9.424 | 1.423 | −14.448 | 0.00 | 0.00 | 0.158 | 9.40 | 4.00 |
| ATOM | 3127 | HA | TYR | 518 | 9.593 | 2.238 | −13.744 | 0.00 | 0.00 | 0.053 | 0.00 | 0.00 |
| ATOM | 3128 | C | TYR | 518 | 10.110 | 1.613 | −15.766 | 0.00 | 0.00 | 0.396 | 9.82 | 4.00 |
| ATOM | 3129 | O | TYR | 518 | 9.489 | 1.447 | −16.815 | 0.00 | 0.00 | −0.396 | 8.17 | −17.40 |
| ATOM | 3130 | CB | TYR | 518 | 9.920 | 0.162 | −13.707 | 0.00 | 0.00 | −0.106 | 12.77 | 4.00 |
| ATOM | 3131 | HB1 | TYR | 518 | 9.265 | −0.145 | −12.892 | 0.00 | 0.00 | 0.053 | 0.00 | 0.00 |
| ATOM | 3132 | HB2 | TYR | 518 | 10.904 | 0.286 | −13.255 | 0.00 | 0.00 | 0.053 | 0.00 | 0.00 |
| ATOM | 3133 | CG | TYR | 518 | 10.038 | −1.028 | −14.587 | 0.00 | 0.00 | 0.000 | 7.26 | 0.60 |
| ATOM | 3134 | CD1 | TYR | 518 | 8.938 | −1.761 | −14.960 | 0.00 | 0.00 | −0.127 | 10.80 | 0.60 |
| ATOM | 3135 | HD1 | TYR | 518 | 7.947 | −1.464 | −14.617 | 0.00 | 0.00 | 0.127 | 0.00 | 0.00 |
| ATOM | 3136 | CD2 | TYR | 518 | 11.283 | −1.429 | −15.014 | 0.00 | 0.00 | −0.127 | 10.80 | 0.60 |
| ATOM | 3137 | HD2 | TYR | 518 | 12.163 | −0.862 | −14.711 | 0.00 | 0.00 | 0.127 | 0.00 | 0.00 |
| ATOM | 3138 | CE1 | TYR | 518 | 9.081 | −2.867 | −15.763 | 0.00 | 0.00 | −0.127 | 10.80 | 0.60 |
| ATOM | 3139 | HE1 | TYR | 518 | 8.203 | −3.440 | −16.060 | 0.00 | 0.00 | 0.127 | 0.00 | 0.00 |
| ATOM | 3140 | CE2 | TYR | 518 | 11.432 | −2.532 | −15.816 | 0.00 | 0.00 | −0.127 | 10.80 | 0.60 |
| ATOM | 3141 | HE2 | TYR | 518 | 12.424 | −2.834 | −16.153 | 0.00 | 0.00 | 0.127 | 0.00 | 0.00 |
| ATOM | 3142 | CZ | TYR | 518 | 10.327 | −3.253 | −16.192 | 0.00 | 0.00 | 0.027 | 7.26 | 0.60 |
| ATOM | 3143 | OH | TYR | 518 | 10.474 | −4.390 | −17.015 | 0.00 | 0.00 | −0.451 | 10.94 | −17.40 |
| ATOM | 3144 | HH | TYR | 518 | 9.728 | −4.400 | −17.725 | 0.00 | 0.00 | 0.424 | 0.00 | 0.00 |
| ATOM | 3145 | N | GLU | 519 | 11.414 | 1.944 | −15.752 | 0.00 | 0.00 | −0.650 | 9.00 | −17.40 |
| ATOM | 3146 | HN | GLU | 519 | 11.988 | 1.786 | −14.912 | 0.00 | 0.00 | 0.439 | 0.00 | 0.00 |
| ATOM | 3147 | CA | GLU | 519 | 11.987 | 2.522 | −16.935 | 0.00 | 0.00 | 0.158 | 9.40 | 4.00 |
| ATOM | 3148 | HA | GLU | 519 | 11.200 | 2.600 | −17.686 | 0.00 | 0.00 | 0.053 | 0.00 | 0.00 |
| ATOM | 3149 | C | GLU | 519 | 13.109 | 1.755 | −17.551 | 0.00 | 0.00 | 0.396 | 9.82 | 4.00 |
| ATOM | 3150 | O | GLU | 519 | 13.365 | 0.589 | −17.245 | 0.00 | 0.00 | −0.396 | 8.17 | −17.40 |
| ATOM | 3151 | CB | GLU | 519 | 12.560 | 3.907 | −16.601 | 0.00 | 0.00 | −0.106 | 12.77 | 4.00 |
| ATOM | 3152 | HB1 | GLU | 519 | 11.774 | 4.585 | −16.267 | 0.00 | 0.00 | 0.053 | 0.00 | 0.00 |
| ATOM | 3153 | HB2 | GLU | 519 | 13.038 | 4.353 | −17.473 | 0.00 | 0.00 | 0.053 | 0.00 | 0.00 |
| ATOM | 3154 | CG | GLU | 519 | 13.607 | 3.814 | −15.486 | 0.00 | 0.00 | −0.336 | 12.77 | 4.00 |
| ATOM | 3155 | HG1 | GLU | 519 | 14.547 | 3.458 | −15.908 | 0.00 | 0.00 | 0.053 | 0.00 | 0.00 |
| ATOM | 3156 | HG2 | GLU | 519 | 13.256 | 3.118 | −14.725 | 0.00 | 0.00 | 0.053 | 0.00 | 0.00 |
| ATOM | 3157 | CD | GLU | 519 | 13.805 | 5.191 | −14.874 | 0.00 | 0.00 | 0.297 | 9.82 | 4.00 |
| ATOM | 3158 | OE1 | GLU | 519 | 13.847 | 6.185 | −15.644 | 0.00 | 0.00 | −0.534 | 8.17 | −18.95 |
| ATOM | 3159 | OE2 | GLU | 519 | 13.911 | 5.267 | −13.621 | 0.00 | 0.00 | −0.534 | 8.17 | −18.95 |
| ATOM | 3160 | N | GLY | 520 | 13.758 | 2.468 | −18.506 | 0.00 | 0.00 | −0.650 | 9.00 | −17.40 |
| ATOM | 3161 | HN | GLY | 520 | 13.389 | 3.411 | −18.693 | 0.00 | 0.00 | 0.439 | 0.00 | 0.00 |
| ATOM | 3162 | CA | GLY | 520 | 14.894 | 2.072 | −19.288 | 0.00 | 0.00 | 0.105 | 9.40 | 4.00 |
| ATOM | 3163 | HA1 | GLY | 520 | 15.654 | 1.694 | −18.604 | 0.00 | 0.00 | 0.053 | 0.00 | 0.00 |
| ATOM | 3164 | HA2 | GLY | 520 | 15.251 | 2.951 | −19.825 | 0.00 | 0.00 | 0.053 | 0.00 | 0.00 |
| ATOM | 3165 | C | GLY | 520 | 14.449 | 1.014 | −20.230 | 0.00 | 0.00 | 0.396 | 9.82 | 4.00 |
| ATOM | 3166 | O | GLY | 520 | 13.300 | 0.962 | −20.658 | 0.00 | 0.00 | −0.396 | 8.17 | −17.40 |
| ATOM | 3167 | N | PRO | 521 | 15.390 | 0.170 | −20.559 | 0.00 | 0.00 | −0.422 | 9.00 | −17.40 |
| ATOM | 3168 | CA | PRO | 521 | 15.083 | −1.006 | −21.334 | 0.00 | 0.00 | 0.158 | 9.40 | 4.00 |
| ATOM | 3169 | HA | PRO | 521 | 14.319 | −0.669 | −22.034 | 0.00 | 0.00 | 0.053 | 0.00 | 0.00 |
| ATOM | 3170 | CD | PRO | 521 | 16.717 | 0.685 | −20.862 | 0.00 | 0.00 | 0.105 | 12.77 | 4.00 |
| ATOM | 3171 | HD1 | PRO | 521 | 17.307 | 0.520 | −19.961 | 0.00 | 0.00 | 0.053 | 0.00 | 0.00 |
| ATOM | 3172 | HD2 | PRO | 521 | 16.575 | 1.741 | −21.093 | 0.00 | 0.00 | 0.053 | 0.00 | 0.00 |
| ATOM | 3173 | C | PRO | 521 | 14.597 | −1.993 | −20.320 | 0.00 | 0.00 | 0.396 | 9.82 | 4.00 |
| ATOM | 3174 | O | PRO | 521 | 14.352 | −3.148 | −20.652 | 0.00 | 0.00 | −0.396 | 8.17 | −17.40 |
| ATOM | 3175 | CB | PRO | 521 | 16.391 | −1.431 | −21.996 | 0.00 | 0.00 | −0.106 | 12.77 | 4.00 |
| ATOM | 3176 | HB1 | PRO | 521 | 16.208 | −1.842 | −22.989 | 0.00 | 0.00 | 0.053 | 0.00 | 0.00 |
| ATOM | 3177 | HB2 | PRO | 521 | 16.897 | −2.193 | −21.403 | 0.00 | 0.00 | 0.053 | 0.00 | 0.00 |
| ATOM | 3178 | CG | PRO | 521 | 17.211 | −0.133 | −22.064 | 0.00 | 0.00 | −0.106 | 12.77 | 4.00 |
| ATOM | 3179 | HG1 | PRO | 521 | 16.953 | 0.294 | −23.033 | 0.00 | 0.00 | 0.053 | 0.00 | 0.00 |
| ATOM | 3180 | HG2 | PRO | 521 | 18.245 | −0.458 | −21.983 | 0.00 | 0.00 | 0.053 | 0.00 | 0.00 |
| ATOM | 3181 | N | ALA | 522 | 14.493 | −1.517 | −19.069 | 0.00 | 0.00 | −0.650 | 9.00 | −17.40 |
| ATOM | 3182 | HN | ALA | 522 | 14.727 | −0.519 | −18.968 | 0.00 | 0.00 | 0.439 | 0.00 | 0.00 |
| ATOM | 3183 | CA | ALA | 522 | 14.113 | −2.164 | −17.860 | 0.00 | 0.00 | 0.158 | 9.40 | 4.00 |
| ATOM | 3184 | HA | ALA | 522 | 13.342 | −1.541 | −17.407 | 0.00 | 0.00 | 0.053 | 0.00 | 0.00 |
| ATOM | 3185 | C | ALA | 522 | 15.352 | −2.245 | −17.015 | 0.00 | 0.00 | 0.396 | 9.82 | 4.00 |
| ATOM | 3186 | O | ALA | 522 | 15.721 | −3.359 | −16.646 | 0.00 | 0.00 | −0.396 | 8.17 | −17.40 |
| ATOM | 3187 | CB | ALA | 522 | 13.588 | −3.596 | −18.059 | 0.00 | 0.00 | −0.159 | 16.15 | 4.00 |
| ATOM | 3188 | HB1 | ALA | 522 | 13.319 | −4.022 | −17.093 | 0.00 | 0.00 | 0.053 | 0.00 | 0.00 |
| ATOM | 3189 | HB2 | ALA | 522 | 12.709 | −3.575 | −18.704 | 0.00 | 0.00 | 0.053 | 0.00 | 0.00 |
| ATOM | 3190 | HB3 | ALA | 522 | 14.363 | −4.207 | −18.522 | 0.00 | 0.00 | 0.053 | 0.00 | 0.00 |
| ATOM | 3191 | N | PRO | 523 | 16.066 | −1.182 | −16.680 | 0.00 | 0.00 | −0.422 | 9.00 | −17.40 |
| ATOM | 3192 | CA | PRO | 523 | 17.192 | −1.369 | −15.818 | 0.00 | 0.00 | 0.158 | 9.40 | 4.00 |
| ATOM | 3193 | HA | PRO | 523 | 17.738 | −2.247 | −16.163 | 0.00 | 0.00 | 0.053 | 0.00 | 0.00 |
| ATOM | 3194 | CD | PRO | 523 | 16.160 | 0.085 | −17.393 | 0.00 | 0.00 | 0.105 | 12.77 | 4.00 |
| ATOM | 3195 | HD1 | PRO | 523 | 15.146 | 0.486 | −17.397 | 0.00 | 0.00 | 0.053 | 0.00 | 0.00 |
| ATOM | 3196 | HD2 | PRO | 523 | 16.527 | −0.173 | −18.386 | 0.00 | 0.00 | 0.053 | 0.00 | 0.00 |
| ATOM | 3197 | C | PRO | 523 | 16.685 | −1.569 | −14.441 | 0.00 | 0.00 | 0.396 | 9.82 | 4.00 |
| ATOM | 3198 | O | PRO | 523 | 17.374 | −2.184 | −13.629 | 0.00 | 0.00 | −0.396 | 8.17 | −17.40 |
| ATOM | 3199 | CB | PRO | 523 | 18.069 | −0.124 | −15.952 | 0.00 | 0.00 | −0.106 | 12.77 | 4.00 |
| ATOM | 3200 | HB1 | PRO | 523 | 18.889 | −0.435 | −16.599 | 0.00 | 0.00 | 0.053 | 0.00 | 0.00 |
| ATOM | 3201 | HB2 | PRO | 523 | 18.375 | 0.106 | −14.931 | 0.00 | 0.00 | 0.053 | 0.00 | 0.00 |
| ATOM | 3202 | CG | PRO | 523 | 17.147 | 0.931 | −16.579 | 0.00 | 0.00 | −0.106 | 12.77 | 4.00 |
| ATOM | 3203 | HG1 | PRO | 523 | 17.709 | 1.620 | −17.209 | 0.00 | 0.00 | 0.053 | 0.00 | 0.00 |
| ATOM | 3204 | HG2 | PRO | 523 | 16.643 | 1.519 | −15.812 | 0.00 | 0.00 | 0.053 | 0.00 | 0.00 |
| ATOM | 3205 | N | LEU | 524 | 15.483 | −1.043 | −14.147 | 0.00 | 0.00 | −0.650 | 9.00 | −17.40 |
| ATOM | 3206 | HN | LEU | 524 | 14.912 | −0.561 | −14.855 | 0.00 | 0.00 | 0.439 | 0.00 | 0.00 |
| ATOM | 3207 | CA | LEU | 524 | 15.045 | −1.191 | −12.800 | 0.00 | 0.00 | 0.158 | 9.40 | 4.00 |
| ATOM | 3208 | HA | LEU | 524 | 15.829 | −0.758 | −12.179 | 0.00 | 0.00 | 0.053 | 0.00 | 0.00 |
| ATOM | 3209 | C | LEU | 524 | 14.880 | −2.652 | −12.580 | 0.00 | 0.00 | 0.396 | 9.82 | 4.00 |
| ATOM | 3210 | O | LEU | 524 | 15.391 | −3.211 | −11.611 | 0.00 | 0.00 | −0.396 | 8.17 | −17.40 |
| ATOM | 3211 | CB | LEU | 524 | 13.706 | −0.491 | −12.500 | 0.00 | 0.00 | −0.106 | 12.77 | 4.00 |
| ATOM | 3212 | HB1 | LEU | 524 | 12.881 | −0.865 | −13.106 | 0.00 | 0.00 | 0.053 | 0.00 | 0.00 |
| ATOM | 3213 | HB2 | LEU | 524 | 13.734 | 0.584 | −12.677 | 0.00 | 0.00 | 0.053 | 0.00 | 0.00 |
| ATOM | 3214 | CG | LEU | 524 | 13.249 | −0.655 | −11.036 | 0.00 | 0.00 | −0.053 | 9.40 | 4.00 |
| ATOM | 3215 | HG | LEU | 524 | 13.144 | −1.708 | −10.774 | 0.00 | 0.00 | 0.053 | 0.00 | 0.00 |
| ATOM | 3216 | CD1 | LEU | 524 | 14.263 | −0.028 | −10.067 | 0.00 | 0.00 | −0.159 | 16.15 | 4.00 |
| ATOM | 3217 | HD11 | LEU | 524 | 13.915 | −0.158 | −9.042 | 0.00 | 0.00 | 0.053 | 0.00 | 0.00 |
| ATOM | 3218 | HD12 | LEU | 524 | 15.230 | −0.516 | −10.187 | 0.00 | 0.00 | 0.053 | 0.00 | 0.00 |
| ATOM | 3219 | HD13 | LEU | 524 | 14.364 | 1.036 | −10.284 | 0.00 | 0.00 | 0.053 | 0.00 | 0.00 |
| ATOM | 3220 | CD2 | LEU | 524 | 11.828 | −0.108 | −10.827 | 0.00 | 0.00 | −0.159 | 16.15 | 4.00 |
| ATOM | 3221 | HD21 | LEU | 524 | 11.537 | −0.239 | −9.785 | 0.00 | 0.00 | 0.053 | 0.00 | 0.00 |
| ATOM | 3222 | HD22 | LEU | 524 | 11.805 | 0.952 | −11.079 | 0.00 | 0.00 | 0.053 | 0.00 | 0.00 |
| ATOM | 3223 | HD23 | LEU | 524 | 11.132 | −0.648 | −11.469 | 0.00 | 0.00 | 0.053 | 0.00 | 0.00 |
| ATOM | 3224 | N | GLU | 525 | 14.216 | −3.321 | −13.540 | 0.00 | 0.00 | −0.650 | 9.00 | −17.40 |
| ATOM | 3225 | HN | GLU | 525 | 13.904 | −2.822 | −14.385 | 0.00 | 0.00 | 0.439 | 0.00 | 0.00 |
| ATOM | 3226 | CA | GLU | 525 | 13.938 | −4.719 | −13.400 | 0.00 | 0.00 | 0.158 | 9.40 | 4.00 |
| ATOM | 3227 | HA | GLU | 525 | 13.360 | −4.871 | −12.488 | 0.00 | 0.00 | 0.053 | 0.00 | 0.00 |
| ATOM | 3228 | C | GLU | 525 | 15.233 | −5.460 | −13.320 | 0.00 | 0.00 | 0.396 | 9.82 | 4.00 |
| ATOM | 3229 | O | GLU | 525 | 15.414 | −6.324 | −12.463 | 0.00 | 0.00 | −0.396 | 8.17 | −17.40 |
| ATOM | 3230 | CB | GLU | 525 | 13.136 | −5.282 | −14.591 | 0.00 | 0.00 | −0.106 | 12.77 | 4.00 |
| ATOM | 3231 | HB1 | GLU | 525 | 13.784 | −5.285 | −15.468 | 0.00 | 0.00 | 0.053 | 0.00 | 0.00 |
| ATOM | 3232 | HB2 | GLU | 525 | 12.271 | −4.639 | −14.754 | 0.00 | 0.00 | 0.053 | 0.00 | 0.00 |
| ATOM | 3233 | CG | GLU | 525 | 12.620 | −6.712 | −14.391 | 0.00 | 0.00 | −0.336 | 12.77 | 4.00 |
| ATOM | 3234 | HG1 | GLU | 525 | 11.680 | −6.833 | −14.929 | 0.00 | 0.00 | 0.053 | 0.00 | 0.00 |
| ATOM | 3235 | HG2 | GLU | 525 | 12.461 | −6.891 | −13.328 | 0.00 | 0.00 | 0.053 | 0.00 | 0.00 |
| ATOM | 3236 | CD | GLU | 525 | 13.650 | −7.698 | −14.929 | 0.00 | 0.00 | 0.297 | 9.82 | 4.00 |
| ATOM | 3237 | OE1 | GLU | 525 | 14.046 | −7.552 | −16.116 | 0.00 | 0.00 | −0.534 | 8.17 | −18.95 |
| ATOM | 3238 | OE2 | GLU | 525 | 14.050 | −8.612 | −14.160 | 0.00 | 0.00 | −0.534 | 8.17 | −18.95 |
| ATOM | 3239 | N | ALA | 526 | 16.182 | −5.111 | −14.204 | 0.00 | 0.00 | −0.650 | 9.00 | −17.40 |
| ATOM | 3240 | HN | ALA | 526 | 16.014 | −4.326 | −14.849 | 0.00 | 0.00 | 0.439 | 0.00 | 0.06 |
| ATOM | 3241 | CA | ALA | 526 | 17.425 | −5.820 | −14.259 | 0.00 | 0.00 | 0.158 | 9.40 | 4.00 |
| ATOM | 3242 | HA | ALA | 526 | 17.254 | −6.886 | −14.410 | 0.00 | 0.00 | 0.053 | 0.00 | 0.00 |
| ATOM | 3243 | C | ALA | 526 | 18.190 | −5.644 | −12.986 | 0.00 | 0.00 | 0.396 | 9.82 | 4.00 |
| ATOM | 3244 | O | ALA | 526 | 18.708 | −6.613 | −12.434 | 0.00 | 0.00 | −0.396 | 8.17 | −17.40 |
| ATOM | 3245 | CB | ALA | 526 | 18.333 | −5.339 | −15.404 | 0.00 | 0.00 | −0.159 | 16.15 | 4.00 |
| ATOM | 3246 | HB1 | ALA | 526 | 19.261 | −5.911 | −15.398 | 0.00 | 0.00 | 0.053 | 0.00 | 0.00 |
| ATOM | 3247 | HB2 | ALA | 526 | 17.824 | −5.484 | −16.357 | 0.00 | 0.00 | 0.053 | 0.00 | 0.00 |
| ATOM | 3248 | HB3 | ALA | 526 | 18.558 | −4.281 | −15.270 | 0.00 | 0.00 | 0.053 | 0.00 | 0.00 |
| ATOM | 3249 | N | ILE | 527 | 18.257 | −4.407 | −12.461 | 0.00 | 0.00 | −0.650 | 9.00 | −17.40 |
| ATOM | 3250 | HN | ILE | 527 | 17.703 | −3.635 | −12.857 | 0.00 | 0.00 | 0.439 | 0.00 | 0.00 |
| ATOM | 3251 | CA | ILE | 527 | 19.119 | −4.191 | −11.337 | 0.00 | 0.00 | 0.158 | 9.40 | 4.00 |
| ATOM | 3252 | HA | ILE | 527 | 20.079 | −4.689 | −11.473 | 0.00 | 0.00 | 0.053 | 0.00 | 0.00 |
| ATOM | 3253 | C | ILE | 527 | 18.513 | −4.716 | −10.082 | 0.00 | 0.00 | 0.396 | 9.82 | 4.00 |
| ATOM | 3254 | O | ILE | 527 | 17.296 | −4.739 | −9.906 | 0.00 | 0.00 | −0.396 | 8.17 | −17.40 |
| ATOM | 3255 | CB | ILE | 527 | 19.467 | −2.752 | −11.092 | 0.00 | 0.00 | −0.053 | 9.40 | 4.00 |
| ATOM | 3256 | HB | ILE | 527 | 20.095 | −2.707 | −10.202 | 0.00 | 0.00 | 0.053 | 0.00 | 0.00 |
| ATOM | 3257 | CG1 | ILE | 527 | 18.213 | −1.937 | −10.739 | 0.00 | 0.00 | −0.106 | 12.77 | 4.00 |
| ATOM | 3258 | HG11 | ILE | 527 | 17.578 | −2.425 | −9.999 | 0.00 | 0.00 | 0.053 | 0.00 | 0.00 |
| ATOM | 3259 | HG12 | ILE | 527 | 17.568 | −1.748 | −11.597 | 0.00 | 0.00 | 0.053 | 0.00 | 0.00 |
| ATOM | 3260 | CG2 | ILE | 527 | 20.221 | −2.233 | −12.327 | 0.00 | 0.00 | −0.159 | 16.15 | 4.00 |
| ATOM | 3261 | HG21 | ILE | 527 | 20.488 | −1.187 | −12.177 | 0.00 | 0.00 | 0.053 | 0.00 | 0.00 |
| ATOM | 3262 | HG22 | ILE | 527 | 21.127 | −2.821 | −12.474 | 0.00 | 0.00 | 0.053 | 0.00 | 0.00 |
| ATOM | 3263 | HG23 | ILE | 527 | 19.584 | −2.323 | −13.207 | 0.00 | 0.00 | 0.053 | 0.00 | 0.00 |
| ATOM | 3264 | CD1 | ILE | 527 | 18.534 | −0.561 | −10.158 | 0.00 | 0.00 | −0.159 | 16.15 | 4.00 |
| ATOM | 3265 | HD11 | ILE | 527 | 17.606 | −0.037 | −9.930 | 0.00 | 0.00 | 0.053 | 0.00 | 0.00 |
| ATOM | 3266 | HD12 | ILE | 527 | 19.118 | −0.679 | −9.245 | 0.00 | 0.00 | 0.053 | 0.00 | 0.00 |
| ATOM | 3267 | HD13 | ILE | 527 | 19.108 | 0.016 | −10.884 | 0.00 | 0.00 | 0.053 | 0.00 | 0.00 |
| ATOM | 3268 | N | ALA | 528 | 19.396 | −5.184 | −9.180 | 0.00 | 0.00 | −0.650 | 9.00 | −17.40 |
| ATOM | 3269 | HN | ALA | 528 | 20.394 | −5.201 | −9.433 | 0.00 | 0.00 | 0.439 | 0.00 | 0.00 |
| ATOM | 3270 | CA | ALA | 528 | 19.012 | −5.658 | −7.887 | 0.00 | 0.00 | 0.158 | 9.40 | 4.00 |
| ATOM | 3271 | HA | ALA | 528 | 18.120 | −5.090 | −7.623 | 0.00 | 0.00 | 0.053 | 0.00 | 0.00 |
| ATOM | 3272 | C | ALA | 528 | 20.176 | −5.371 | −6.997 | 0.00 | 0.00 | 0.396 | 9.82 | 4.00 |
| ATOM | 3273 | O | ALA | 528 | 21.257 | −5.929 | −7.180 | 0.00 | 0.00 | −0.396 | 8.17 | −17.40 |
| ATOM | 3274 | CB | ALA | 528 | 18.768 | −7.175 | −7.834 | 0.00 | 0.00 | −0.159 | 16.15 | 4.00 |
| ATOM | 3275 | HB1 | ALA | 528 | 18.480 | −7.462 | −6.823 | 0.00 | 0.00 | 0.053 | 0.00 | 0.00 |
| ATOM | 3276 | HB2 | ALA | 528 | 17.969 | −7.440 | −8.527 | 0.00 | 0.00 | 0.053 | 0.00 | 0.00 |
| ATOM | 3277 | HB3 | ALA | 528 | 19.681 | −7.700 | −8.115 | 0.00 | 0.00 | 0.053 | 0.00 | 0.00 |
| ATOM | 3278 | N | ASN | 529 | 19.996 | −4.469 | −6.013 | 0.00 | 0.00 | −0.650 | 9.00 | −17.40 |
| ATOM | 3279 | HN | ASN | 529 | 19.086 | −4.002 | −5.892 | 0.00 | 0.00 | 0.439 | 0.00 | 0.00 |
| ATOM | 3280 | CA | ASN | 529 | 21.089 | −4.169 | −5.138 | 0.00 | 0.00 | 0.158 | 9.40 | 4.00 |
| ATOM | 3281 | HA | ASN | 529 | 21.866 | −4.911 | −5.321 | 0.00 | 0.00 | 0.053 | 0.00 | 0.00 |
| ATOM | 3282 | C | ASN | 529 | 20.576 | −4.249 | −3.740 | 0.00 | 0.00 | 0.396 | 9.82 | 4.00 |
| ATOM | 3283 | O | ASN | 529 | 19.423 | −3.921 | −3.469 | 0.00 | 0.00 | −0.396 | 8.17 | −17.40 |
| ATOM | 3284 | CB | ASN | 529 | 21.674 | −2.759 | −5.344 | 0.00 | 0.00 | −0.106 | 12.77 | 4.00 |
| ATOM | 3285 | HB1 | ASN | 529 | 20.933 | −2.024 | −5.030 | 0.00 | 0.00 | 0.053 | 0.00 | 0.00 |
| ATOM | 3286 | HB2 | ASN | 529 | 21.910 | −2.629 | −6.400 | 0.00 | 0.00 | 0.053 | 0.00 | 0.00 |
| ATOM | 3287 | CG | ASN | 529 | 22.938 | −2.626 | −4.501 | 0.00 | 0.00 | 0.396 | 9.82 | 4.00 |
| ATOM | 3288 | OD1 | ASN | 529 | 22.887 | −2.582 | −3.273 | 0.00 | 0.00 | −0.396 | 8.17 | −17.40 |
| ATOM | 3289 | ND2 | ASN | 529 | 24.112 | −2.560 | −5.184 | 0.00 | 0.00 | −0.879 | 13.25 | −17.40 |
| ATOM | 3290 | HD21 | ASN | 529 | 24.112 | −2.600 | −6.213 | 0.00 | 0.00 | 0.439 | 0.00 | 0.00 |
| ATOM | 3291 | HD22 | ASN | 529 | 25.001 | −2.470 | −4.672 | 0.00 | 0.00 | 0.439 | 0.00 | 0.00 |
| ATOM | 3292 | N | GLY | 530 | 21.433 | −4.714 | −2.812 | 0.00 | 0.00 | −0.650 | 9.00 | −17.40 |
| ATOM | 3293 | HN | GLY | 530 | 22.382 | −5.006 | −3.088 | 0.00 | 0.00 | 0.439 | 0.00 | 0.00 |
| ATOM | 3294 | CA | GLY | 530 | 21.027 | −4.803 | −1.444 | 0.00 | 0.00 | 0.105 | 9.40 | 4.00 |
| ATOM | 3295 | HA1 | GLY | 530 | 20.191 | −4.118 | −1.299 | 0.00 | 0.00 | 0.053 | 0.00 | 0.00 |
| ATOM | 3296 | HA2 | GLY | 530 | 21.878 | −4.519 | −0.825 | 0.00 | 0.00 | 0.053 | 0.00 | 0.00 |
| ATOM | 3297 | C | GLY | 530 | 20.618 | −6.212 | −1.181 | 0.00 | 0.00 | 0.396 | 9.82 | 4.00 |
| ATOM | 3298 | O | GLY | 530 | 21.190 | −7.155 | −1.727 | 0.00 | 0.00 | −0.396 | 8.17 | −17.40 |
| ATOM | 3299 | N | CYS | 531 | 19.602 | −6.380 | −0.316 | 0.00 | 0.00 | −0.650 | 9.00 | −17.40 |
| ATOM | 3300 | HN | CYS | 531 | 19.159 | −5.553 | 0.109 | 0.00 | 0.00 | 0.439 | 0.00 | 0.00 |
| ATOM | 3301 | CA | CYS | 531 | 19.125 | −7.684 | 0.025 | 0.00 | 0.00 | 0.158 | 9.40 | 4.00 |
| ATOM | 3302 | HA | CYS | 531 | 19.987 | −8.351 | 0.042 | 0.00 | 0.00 | 0.053 | 0.00 | 0.00 |
| ATOM | 3303 | C | CYS | 531 | 18.144 | −8.086 | −1.021 | 0.00 | 0.00 | 0.396 | 9.82 | 4.00 |
| ATOM | 3304 | O | CYS | 531 | 18.365 | −7.878 | −2.213 | 0.00 | 0.00 | −0.396 | 8.17 | −17.40 |
| ATOM | 3305 | CB | CYS | 531 | 18.404 | −7.733 | 1.382 | 0.00 | 0.00 | −0.041 | 12.77 | 4.00 |
| ATOM | 3306 | HB1 | CYS | 531 | 17.978 | −8.714 | 1.591 | 0.00 | 0.00 | 0.053 | 0.00 | 0.00 |
| ATOM | 3307 | HB2 | CYS | 531 | 17.581 | −7.021 | 1.441 | 0.00 | 0.00 | 0.053 | 0.00 | 0.00 |
| ATOM | 3308 | SG | CYS | 531 | 19.514 | −7.350 | 2.771 | 0.00 | 0.00 | −0.255 | 19.93 | −6.40 |
| ATOM | 3309 | HG | CYS | 531 | 20.053 | −6.136 | 2.591 | 0.00 | 0.00 | 0.190 | 0.00 | 0.00 |
| ATOM | 3310 | N | ALA | 532 | 17.025 | −8.696 | −0.590 | 0.00 | 0.00 | −0.650 | 9.00 | −17.40 |
| ATOM | 3311 | HN | ALA | 532 | 16.885 | −8.855 | 0.418 | 0.00 | 0.00 | 0.439 | 0.00 | 0.00 |
| ATOM | 3312 | CA | ALA | 532 | 16.029 | −9.125 | −1.523 | 0.00 | 0.00 | 0.158 | 9.40 | 4.00 |
| ATOM | 3313 | HA | ALA | 532 | 16.493 | −9.854 | −2.187 | 0.00 | 0.00 | 0.053 | 0.00 | 0.00 |
| ATOM | 3314 | C | ALA | 532 | 15.570 | −7.922 | −2.275 | 0.00 | 0.00 | 0.396 | 9.82 | 4.00 |
| ATOM | 3315 | O | ALA | 532 | 14.952 | −7.031 | −1.697 | 0.00 | 0.00 | −0.396 | 8.17 | −17.40 |
| ATOM | 3316 | CB | ALA | 532 | 14.798 | −9.756 | −0.848 | 0.00 | 0.00 | −0.159 | 16.15 | 4.00 |
| ATOM | 3317 | HB1 | ALA | 532 | 14.081 | −10.060 | −1.610 | 0.00 | 0.00 | 0.053 | 0.00 | 0.00 |
| ATOM | 3318 | HB2 | ALA | 532 | 15.106 | −10.628 | −0.272 | 0.00 | 0.00 | 0.053 | 0.00 | 0.00 |
| ATOM | 3319 | HB3 | ALA | 532 | 14.334 | −9.027 | −0.183 | 0.00 | 0.00 | 0.053 | 0.00 | 0.00 |
| ATOM | 3320 | N | PHE | 533 | 15.865 | −7.881 | −3.591 | 0.00 | 0.00 | −0.650 | 9.00 | −17.40 |
| ATOM | 3321 | HN | PHE | 533 | 16.350 | −8.684 | −4.017 | 0.00 | 0.00 | 0.439 | 0.00 | 0.00 |
| ATOM | 3322 | CA | PHE | 533 | 15.528 | −6.762 | −4.415 | 0.00 | 0.00 | 0.158 | 9.40 | 4.00 |
| ATOM | 3323 | HA | PHE | 533 | 14.682 | −6.206 | −4.011 | 0.00 | 0.00 | 0.053 | 0.00 | 0.00 |
| ATOM | 3324 | C | PHE | 533 | 15.156 | −7.190 | −5.802 | 0.00 | 0.00 | 0.396 | 9.82 | 4.00 |
| ATOM | 3325 | O | PHE | 533 | 15.049 | −8.379 | −6.093 | 0.00 | 0.00 | −0.396 | 8.17 | −17.40 |
| ATOM | 3326 | CB | PHE | 533 | 16.585 | −5.658 | −4.423 | 0.00 | 0.00 | −0.106 | 12.77 | 4.00 |
| ATOM | 3327 | HB1 | PHE | 533 | 16.373 | −4.902 | −5.179 | 0.00 | 0.00 | 0.053 | 0.00 | 0.00 |
| ATOM | 3328 | HB2 | PHE | 533 | 17.579 | −6.053 | −4.634 | 0.00 | 0.00 | 0.053 | 0.00 | 0.00 |
| ATOM | 3329 | CG | PHE | 533 | 16.452 | −5.140 | −3.035 | 0.00 | 0.00 | 0.000 | 7.26 | 0.60 |
| ATOM | 3330 | CD1 | PHE | 533 | 15.291 | −4.498 | −2.661 | 0.00 | 0.00 | −0.127 | 10.80 | 0.60 |
| ATOM | 3331 | HD1 | PHE | 533 | 14.486 | −4.375 | −3.386 | 0.00 | 0.00 | 0.127 | 0.00 | 0.00 |
| ATOM | 3332 | CD2 | PHE | 533 | 17.460 | −5.289 | −2.113 | 0.00 | 0.00 | −0.127 | 10.80 | 0.60 |
| ATOM | 3333 | HD2 | PHE | 533 | 18.384 | −5.793 | −2.397 | 0.00 | 0.00 | 0.127 | 0.00 | 0.00 |
| ATOM | 3334 | CE1 | PHE | 533 | 15.136 | −4.012 | −1.387 | 0.00 | 0.00 | −0.127 | 10.80 | 0.60 |
| ATOM | 3335 | HE1 | PHE | 533 | 14.214 | −3.505 | −1.104 | 0.00 | 0.00 | 0.127 | 0.00 | 0.00 |
| ATOM | 3336 | CE2 | PHE | 533 | 17.310 | −4.805 | −0.833 | 0.00 | 0.00 | −0.127 | 10.80 | 0.60 |
| ATOM | 3337 | HE2 | PHE | 533 | 18.114 | −4.928 | −0.107 | 0.00 | 0.00 | 0.127 | 0.00 | 0.00 |
| ATOM | 3338 | CZ | PHE | 533 | 16.147 | −4.167 | −0.470 | 0.00 | 0.00 | −0.127 | 10.80 | 0.60 |
| ATOM | 3339 | HZ | PHE | 533 | 16.028 | −3.785 | 0.544 | 0.00 | 0.00 | 0.127 | 0.00 | 0.00 |
| ATOM | 3340 | N | LEU | 534 | 15.030 | −6.210 | −6.720 | 0.00 | 0.00 | −0.650 | 9.00 | −17.40 |
| ATOM | 3341 | HN | LEU | 534 | 15.502 | −5.305 | −6.580 | 0.00 | 0.00 | 0.439 | 0.00 | 0.00 |
| ATOM | 3342 | CA | LEU | 534 | 14.237 | −6.435 | −7.888 | 0.00 | 0.00 | 0.158 | 9.40 | 4.00 |
| ATOM | 3343 | HA | LEU | 534 | 13.200 | −6.550 | −7.572 | 0.00 | 0.00 | 0.053 | 0.00 | 0.00 |
| ATOM | 3344 | C | LEU | 534 | 14.588 | −7.645 | −8.691 | 0.00 | 0.00 | 0.396 | 9.82 | 4.00 |
| ATOM | 3345 | O | LEU | 534 | 13.808 | −8.593 | −8.683 | 0.00 | 0.00 | −0.396 | 8.17 | −17.40 |
| ATOM | 3346 | CB | LEU | 534 | 14.157 | −5.189 | −8.803 | 0.00 | 0.00 | −0.106 | 12.77 | 4.00 |
| ATOM | 3347 | HB1 | LEU | 534 | 15.066 | −5.154 | −9.404 | 0.00 | 0.00 | 0.053 | 0.00 | 0.00 |
| ATOM | 3348 | HB2 | LEU | 534 | 14.078 | −4.308 | −8.166 | 0.00 | 0.00 | 0.053 | 0.00 | 0.00 |
| ATOM | 3349 | CG | LEU | 534 | 12.955 | −5.157 | −9.784 | 0.00 | 0.00 | −0.053 | 9.40 | 4.00 |
| ATOM | 3350 | HG | LEU | 534 | 12.010 | −5.236 | −9.247 | 0.00 | 0.00 | 0.053 | 0.00 | 0.00 |
| ATOM | 3351 | CD1 | LEU | 534 | 12.934 | −3.848 | −10.586 | 0.00 | 0.00 | −0.159 | 16.15 | 4.00 |
| ATOM | 3352 | HD11 | LEU | 534 | 12.082 | −3.851 | −11.266 | 0.00 | 0.00 | 0.053 | 0.00 | 0.00 |
| ATOM | 3353 | HD12 | LEU | 534 | 12.849 | −3.004 | −9.902 | 0.00 | 0.00 | 0.053 | 0.00 | 0.00 |
| ATOM | 3354 | HD13 | LEU | 534 | 13.856 | −3.758 | −11.160 | 0.00 | 0.00 | 0.053 | 0.00 | 0.00 |
| ATOM | 3355 | CD2 | LEU | 534 | 12.883 | −6.380 | −10.710 | 0.00 | 0.00 | −0.159 | 16.15 | 4.00 |
| ATOM | 3356 | HD21 | LEU | 534 | 12.018 | −6.288 | −11.367 | 0.00 | 0.00 | 0.053 | 0.00 | 0.00 |
| ATOM | 3357 | HD22 | LEU | 534 | 13.791 | −6.436 | −11.311 | 0.00 | 0.00 | 0.053 | 0.00 | 0.00 |
| ATOM | 3358 | HD23 | LEU | 534 | 12.789 | −7.285 | −10.110 | 0.00 | 0.00 | 0.053 | 0.00 | 0.00 |
| ATOM | 3359 | N | ASN | 535 | 15.742 | −7.721 | −9.375 | 0.00 | 0.00 | −0.650 | 9.00 | −17.40 |
| ATOM | 3360 | HN | ASN | 535 | 16.522 | −7.056 | −9.276 | 0.00 | 0.00 | 0.439 | 0.00 | 0.00 |
| ATOM | 3361 | CA | ASN | 535 | 15.745 | −8.850 | −10.262 | 0.00 | 0.00 | 0.158 | 9.40 | 4.00 |
| ATOM | 3362 | HA | ASN | 535 | 14.811 | −8.848 | −10.824 | 0.00 | 0.00 | 0.053 | 0.00 | 0.00 |
| ATOM | 3363 | C | ASN | 535 | 15.858 | −10.190 | −9.557 | 0.00 | 0.00 | 0.396 | 9.82 | 4.00 |
| ATOM | 3364 | O | ASN | 535 | 14.842 | −10.879 | −9.513 | 0.00 | 0.00 | −0.396 | 8.17 | −17.40 |
| ATOM | 3365 | CB | ASN | 535 | 16.754 | −8.685 | −11.423 | 0.00 | 0.00 | −0.106 | 12.77 | 4.00 |
| ATOM | 3366 | HB1 | ASN | 535 | 17.767 | −8.799 | −11.038 | 0.00 | 0.00 | 0.053 | 0.00 | 0.00 |
| ATOM | 3367 | HB2 | ASN | 535 | 16.638 | −7.694 | −11.863 | 0.00 | 0.00 | 0.053 | 0.00 | 0.00 |
| ATOM | 3368 | CG | ASN | 535 | 16.484 | −9.750 | −12.480 | 0.00 | 0.00 | 0.396 | 9.82 | 4.00 |
| ATOM | 3369 | OD1 | ASN | 535 | 15.799 | −10.744 | −12.240 | 0.00 | 0.00 | −0.396 | 8.17 | −17.40 |
| ATOM | 3370 | ND2 | ASN | 535 | 17.052 | −9.540 | −13.697 | 0.00 | 0.00 | −0.879 | 13.25 | −17.40 |
| ATOM | 3371 | HD21 | ASN | 535 | 17.619 | −8.696 | −13.862 | 0.00 | 0.00 | 0.439 | 0.00 | 0.00 |
| ATOM | 3372 | HD22 | ASN | 535 | 16.915 | −10.225 | −14.454 | 0.00 | 0.00 | 0.439 | 0.00 | 0.00 |
| ATOM | 3373 | N | PRO | 536 | 16.947 | −10.662 | −8.978 | 0.00 | 0.00 | −0.422 | 9.00 | −17.40 |
| ATOM | 3374 | CA | PRO | 536 | 16.790 | −11.968 | −8.375 | 0.00 | 0.00 | 0.158 | 9.40 | 4.00 |
| ATOM | 3375 | HA | PRO | 536 | 15.997 | −12.493 | −8.908 | 0.00 | 0.00 | 0.053 | 0.00 | 0.00 |
| ATOM | 3376 | CD | PRO | 536 | 18.244 | −10.566 | −9.640 | 0.00 | 0.00 | 0.105 | 12.77 | 4.00 |
| ATOM | 3377 | HD1 | PRO | 536 | 18.866 | −9.936 | −9.004 | 0.00 | 0.00 | 0.053 | 0.00 | 0.00 |
| ATOM | 3378 | HD2 | PRO | 536 | 18.057 | −10.117 | −10.615 | 0.00 | 0.00 | 0.053 | 0.00 | 0.00 |
| ATOM | 3379 | C | PRO | 536 | 16.423 | −11.965 | −6.925 | 0.00 | 0.00 | 0.396 | 9.82 | 4.00 |
| ATOM | 3380 | O | PRO | 536 | 15.913 | −12.968 | −6.429 | 0.00 | 0.00 | −0.396 | 8.17 | −17.40 |
| ATOM | 3381 | CB | PRO | 536 | 18.109 | −12.713 | −8.571 | 0.00 | 0.00 | −0.106 | 12.77 | 4.00 |
| ATOM | 3382 | HB1 | PRO | 536 | 17.810 | −13.739 | −8.786 | 0.00 | 0.00 | 0.053 | 0.00 | 0.00 |
| ATOM | 3383 | HB2 | PRO | 536 | 18.631 | −12.592 | −7.622 | 0.00 | 0.00 | 0.053 | 0.00 | 0.00 |
| ATOM | 3384 | CG | PRO | 536 | 18.778 | −11.997 | −9.748 | 0.00 | 0.00 | −0.106 | 12.77 | 4.00 |
| ATOM | 3385 | HG1 | PRO | 536 | 18.504 | −12.461 | −10.695 | 0.00 | 0.00 | 0.053 | 0.00 | 0.00 |
| ATOM | 3386 | HG2 | PRO | 536 | 19.864 | −12.031 | −9.661 | 0.00 | 0.00 | 0.053 | 0.00 | 0.00 |
| ATOM | 3387 | N | LYS | 537 | 16.702 | −10.846 | −6.242 | 0.00 | 0.00 | −0.650 | 9.00 | −17.40 |
| ATOM | 3388 | HN | LYS | 537 | 16.889 | −9.980 | −6.767 | 0.00 | 0.00 | 0.439 | 0.00 | 0.00 |
| ATOM | 3389 | CA | LYS | 537 | 16.750 | −10.806 | −4.813 | 0.00 | 0.00 | 0.158 | 9.40 | 4.00 |
| ATOM | 3390 | HA | LYS | 537 | 17.305 | −11.689 | −4.496 | 0.00 | 0.00 | 0.053 | 0.00 | 0.00 |
| ATOM | 3391 | C | LYS | 537 | 15.480 | −10.815 | −3.984 | 0.00 | 0.00 | 0.396 | 9.82 | 4.00 |
| ATOM | 3392 | O | LYS | 537 | 15.499 | −11.419 | −2.914 | 0.00 | 0.00 | −0.396 | 8.17 | −17.40 |
| ATOM | 3393 | CB | LYS | 537 | 17.789 | −9.773 | −4.347 | 0.00 | 0.00 | −0.106 | 12.77 | 4.00 |
| ATOM | 3394 | HB1 | LYS | 537 | 18.781 | −10.207 | −4.224 | 0.00 | 0.00 | 0.053 | 0.00 | 0.00 |
| ATOM | 3395 | HB2 | LYS | 537 | 17.529 | −9.325 | −3.388 | 0.00 | 0.00 | 0.053 | 0.00 | 0.00 |
| ATOM | 3396 | CG | LYS | 537 | 17.946 | −8.613 | −5.337 | 0.00 | 0.00 | −0.106 | 12.77 | 4.00 |
| ATOM | 3397 | HG1 | LYS | 537 | 17.018 | −8.074 | −5.528 | 0.00 | 0.00 | 0.053 | 0.00 | 0.00 |
| ATOM | 3398 | HG2 | LYS | 537 | 18.300 | −8.923 | −6.320 | 0.00 | 0.00 | 0.053 | 0.00 | 0.00 |
| ATOM | 3399 | CD | LYS | 537 | 18.937 | −7.547 | −4.877 | 0.00 | 0.00 | −0.106 | 12.77 | 4.00 |
| ATOM | 3400 | HD1 | LYS | 537 | 18.728 | −7.189 | −3.869 | 0.00 | 0.00 | 0.053 | 0.00 | 0.00 |
| ATOM | 3401 | HD2 | LYS | 537 | 18.932 | −6.667 | −5.520 | 0.00 | 0.00 | 0.053 | 0.00 | 0.00 |
| ATOM | 3402 | CE | LYS | 537 | 20.379 | −8.056 | −4.859 | 0.00 | 0.00 | 0.099 | 12.77 | 4.00 |
| ATOM | 3403 | HE1 | LYS | 537 | 20.676 | −8.386 | −5.854 | 0.00 | 0.00 | 0.053 | 0.00 | 0.00 |
| ATOM | 3404 | HE2 | LYS | 537 | 20.475 | −8.895 | −4.170 | 0.00 | 0.00 | 0.053 | 0.00 | 0.00 |
| ATOM | 3405 | NZ | LYS | 537 | 21.296 | −6.982 | −4.425 | 0.00 | 0.00 | −0.045 | 13.25 | −39.20 |
| ATOM | 3406 | HZ1 | LYS | 537 | 22.263 | −7.338 | −4.417 | 0.00 | 0.00 | 0.280 | 0.00 | 0.00 |
| ATOM | 3407 | HZ2 | LYS | 537 | 21.228 | −6.188 | −5.077 | 0.00 | 0.00 | 0.280 | 0.00 | 0.00 |
| ATOM | 3408 | HZ3 | LYS | 537 | 21.037 | −6.671 | −3.478 | 0.00 | 0.00 | 0.280 | 0.00 | 0.00 |
| ATOM | 3409 | N | PHE | 538 | 14.337 | −10.213 | −4.383 | 0.00 | 0.00 | −0.650 | 9.00 | −17.40 |
| ATOM | 3410 | HN | PHE | 538 | 14.207 | −9.847 | −5.337 | 0.00 | 0.00 | 0.439 | 0.00 | 0.00 |
| ATOM | 3411 | CA | PHE | 538 | 13.311 | −10.123 | −3.368 | 0.00 | 0.00 | 0.158 | 9.40 | 4.00 |
| ATOM | 3412 | HA | PHE | 538 | 13.790 | −10.199 | −2.392 | 0.00 | 0.00 | 0.053 | 0.00 | 0.00 |
| ATOM | 3413 | C | PHE | 538 | 12.260 | −11.195 | −3.440 | 0.00 | 0.00 | 0.396 | 9.82 | 4.00 |
| ATOM | 3414 | O | PHE | 538 | 11.439 | −11.233 | −4.355 | 0.00 | 0.00 | −0.396 | 8.17 | −17.40 |
| ATOM | 3415 | CB | PHE | 538 | 12.599 | −8.750 | −3.335 | 0.00 | 0.00 | −0.106 | 12.77 | 4.00 |
| ATOM | 3416 | HB1 | PHE | 538 | 13.311 | −7.927 | −3.266 | 0.00 | 0.00 | 0.053 | 0.00 | 0.00 |
| ATOM | 3417 | HB2 | PHE | 538 | 11.926 | −8.664 | −2.482 | 0.00 | 0.00 | 0.053 | 0.00 | 0.00 |
| ATOM | 3418 | CG | PHE | 538 | 11.779 | −8.520 | −4.567 | 0.00 | 0.00 | 0.000 | 7.26 | 0.60 |
| ATOM | 3419 | CD1 | PHE | 538 | 12.352 | −8.128 | −5.752 | 0.00 | 0.00 | −0.127 | 10.80 | 0.60 |
| ATOM | 3420 | HD1 | PHE | 538 | 13.431 | −7.978 | −5.800 | 0.00 | 0.00 | 0.127 | 0.00 | 0.00 |
| ATOM | 3421 | CD2 | PHE | 538 | 10.416 | −8.694 | −4.540 | 0.00 | 0.00 | −0.127 | 10.80 | 0.60 |
| ATOM | 3422 | HD2 | PHE | 538 | 9.937 | −9.000 | −3.610 | 0.00 | 0.00 | 0.127 | 0.00 | 0.00 |
| ATOM | 3423 | CE1 | PHE | 538 | 11.592 | −7.921 | −6.880 | 0.00 | 0.00 | −0.127 | 10.80 | 0.60 |
| ATOM | 3424 | HE1 | PHE | 538 | 12.071 | −7.612 | −7.809 | 0.00 | 0.00 | 0.127 | 0.00 | 0.00 |
| ATOM | 3425 | CE2 | PHE | 538 | 9.643 | −8.491 | −5.659 | 0.00 | 0.00 | −0.127 | 10.80 | 0.60 |
| ATOM | 3426 | HE2 | PHE | 538 | 8.564 | −8.638 | −5.611 | 0.00 | 0.00 | 0.127 | 0.00 | 0.00 |
| ATOM | 3427 | CZ | PHE | 538 | 10.231 | −8.103 | −6.837 | 0.00 | 0.00 | −0.127 | 10.80 | 0.60 |
| ATOM | 3428 | HZ | PHE | 538 | 9.625 | −7.941 | −7.728 | 0.00 | 0.00 | 0.127 | 0.00 | 0.00 |
| ATOM | 3429 | N | ASN | 539 | 12.306 | −12.141 | −2.475 | 0.00 | 0.00 | −0.650 | 9.00 | −17.40 |
| ATOM | 3430 | HN | ASN | 539 | 13.124 | −12.169 | −1.849 | 0.00 | 0.00 | 0.439 | 0.00 | 0.00 |
| ATOM | 3431 | CA | ASN | 539 | 11.259 | −13.111 | −2.286 | 0.00 | 0.00 | 0.158 | 9.40 | 4.00 |
| ATOM | 3432 | HA | ASN | 539 | 10.762 | −13.358 | −3.224 | 0.00 | 0.00 | 0.053 | 0.00 | 0.00 |
| ATOM | 3433 | C | ASN | 539 | 10.187 | −12.606 | −1.334 | 0.00 | 0.00 | 0.396 | 9.82 | 4.00 |
| ATOM | 3434 | O | ASN | 539 | 9.033 | −13.001 | −1.492 | 0.00 | 0.00 | −0.396 | 8.17 | −17.40 |
| ATOM | 3435 | CB | ASN | 539 | 11.744 | −14.538 | −1.911 | 0.00 | 0.00 | −0.106 | 12.77 | 4.00 |
| ATOM | 3436 | HB1 | ASN | 539 | 12.365 | −14.996 | −2.681 | 0.00 | 0.00 | 0.053 | 0.00 | 0.00 |
| ATOM | 3437 | HB2 | ASN | 539 | 10.926 | −15.238 | −1.741 | 0.00 | 0.00 | 0.053 | 0.00 | 0.00 |
| ATOM | 3438 | CG | ASN | 539 | 12.579 | −14.572 | −0.641 | 0.00 | 0.00 | 0.396 | 9.82 | 4.00 |
| ATOM | 3439 | OD1 | ASN | 539 | 13.565 | −13.850 | −0.511 | 0.00 | 0.00 | −0.396 | 8.17 | −17.40 |
| ATOM | 3440 | ND2 | ASN | 539 | 12.175 | −15.438 | 0.326 | 0.00 | 0.00 | −0.879 | 13.25 | −17.40 |
| ATOM | 3441 | HD21 | ASN | 539 | 11.342 | −16.025 | 0.176 | 0.00 | 0.00 | 0.439 | 0.00 | 0.00 |
| ATOM | 3442 | HD22 | ASN | 539 | 12.702 | −15.507 | 1.208 | 0.00 | 0.00 | 0.439 | 0.00 | 0.00 |
| ATOM | 3443 | N | PRO | 540 | 10.477 | −11.768 | −0.351 | 0.00 | 0.00 | −0.422 | 9.00 | −17.40 |
| ATOM | 3444 | CA | PRO | 540 | 9.438 | −11.278 | 0.539 | 0.00 | 0.00 | 0.158 | 9.40 | 4.00 |
| ATOM | 3445 | HA | PRO | 540 | 8.633 | −12.008 | 0.624 | 0.00 | 0.00 | 0.053 | 0.00 | 0.00 |
| ATOM | 3446 | CD | PRO | 540 | 11.761 | −11.845 | 0.336 | 0.00 | 0.00 | 0.105 | 12.77 | 4.00 |
| ATOM | 3447 | HD1 | PRO | 540 | 12.499 | −11.415 | −0.341 | 0.00 | 0.00 | 0.053 | 0.00 | 0.00 |
| ATOM | 3448 | HD2 | PRO | 540 | 11.944 | −12.902 | 0.530 | 0.00 | 0.00 | 0.053 | 0.00 | 0.00 |
| ATOM | 3449 | C | PRO | 540 | 8.857 | −9.983 | 0.013 | 0.00 | 0.00 | 0.396 | 9.82 | 4.00 |
| ATOM | 3450 | O | PRO | 540 | 9.441 | −9.505 | −0.957 | 0.00 | 0.00 | −0.396 | 8.17 | −17.40 |
| ATOM | 3451 | CB | PRO | 540 | 10.119 | −11.161 | 1.914 | 0.00 | 0.00 | −0.106 | 12.77 | 4.00 |
| ATOM | 3452 | HB1 | PRO | 540 | 9.861 | −12.077 | 2.445 | 0.00 | 0.00 | 0.053 | 0.00 | 0.00 |
| ATOM | 3453 | HB2 | PRO | 540 | 9.697 | −10.267 | 2.373 | 0.00 | 0.00 | 0.053 | 0.00 | 0.00 |
| ATOM | 3454 | CG | PRO | 540 | 11.612 | −11.037 | 1.620 | 0.00 | 0.00 | −0.106 | 12.77 | 4.00 |
| ATOM | 3455 | HG1 | PRO | 540 | 12.211 | −11.445 | 2.434 | 0.00 | 0.00 | 0.053 | 0.00 | 0.00 |
| ATOM | 3456 | HG2 | PRO | 540 | 11.904 | −9.995 | 1.486 | 0.00 | 0.00 | 0.053 | 0.00 | 0.00 |
| ATOM | 3457 | N | PRO | 541 | 7.809 | −9.385 | 0.595 | 0.00 | 0.00 | −0.422 | 9.00 | −17.40 |
| ATOM | 3458 | CA | PRO | 541 | 7.139 | −8.226 | 0.036 | 0.00 | 0.00 | 0.158 | 9.40 | 4.00 |
| ATOM | 3459 | HA | PRO | 541 | 6.414 | −8.584 | −0.695 | 0.00 | 0.00 | 0.053 | 0.00 | 0.00 |
| ATOM | 3460 | CD | PRO | 541 | 7.567 | −9.460 | 2.032 | 0.00 | 0.00 | 0.105 | 12.77 | 4.00 |
| ATOM | 3461 | HD1 | PRO | 541 | 8.536 | −9.654 | 2.493 | 0.00 | 0.00 | 0.053 | 0.00 | 0.00 |
| ATOM | 3462 | HD2 | PRO | 541 | 6.862 | −10.279 | 2.178 | 0.00 | 0.00 | 0.053 | 0.00 | 0.00 |
| ATOM | 3463 | C | PRO | 541 | 7.993 | −7.223 | −0.664 | 0.00 | 0.00 | 0.396 | 9.82 | 4.00 |
| ATOM | 3464 | O | PRO | 541 | 8.879 | −6.615 | −0.062 | 0.00 | 0.00 | −0.396 | 8.17 | −17.40 |
| ATOM | 3465 | CB | PRO | 541 | 6.303 | −7.605 | 1.156 | 0.00 | 0.00 | −0.106 | 12.77 | 4.00 |
| ATOM | 3466 | HB1 | PRO | 541 | 5.266 | −7.937 | 1.098 | 0.00 | 0.00 | 0.053 | 0.00 | 0.00 |
| ATOM | 3467 | HB2 | PRO | 541 | 6.312 | −6.517 | 1.091 | 0.00 | 0.00 | 0.053 | 0.00 | 0.00 |
| ATOM | 3468 | CG | PRO | 541 | 6.983 | −8.100 | 2.438 | 0.00 | 0.00 | −0.106 | 12.77 | 4.00 |
| ATOM | 3469 | HG1 | PRO | 541 | 6.262 | −8.197 | 3.250 | 0.00 | 0.00 | 0.053 | 0.00 | 0.00 |
| ATOM | 3470 | HG2 | PRO | 541 | 7.760 | −7.407 | 2.760 | 0.00 | 0.00 | 0.053 | 0.00 | 0.00 |
| ATOM | 3471 | N | LYS | 542 | 7.687 | −7.049 | −1.961 | 0.00 | 0.00 | −0.650 | 9.00 | −17.40 |
| ATOM | 3472 | HN | LYS | 542 | 6.865 | −7.555 | −2.319 | 0.00 | 0.00 | 0.439 | 0.00 | 0.00 |
| ATOM | 3473 | CA | LYS | 542 | 8.396 | −6.228 | −2.888 | 0.00 | 0.00 | 0.158 | 9.40 | 4.00 |
| ATOM | 3474 | HA | LYS | 542 | 9.459 | −6.466 | −2.910 | 0.00 | 0.00 | 0.053 | 0.00 | 0.00 |
| ATOM | 3475 | C | LYS | 542 | 8.280 | −4.777 | −2.548 | 0.00 | 0.00 | 0.396 | 9.82 | 4.00 |
| ATOM | 3476 | O | LYS | 542 | 9.266 | −4.049 | −2.623 | 0.00 | 0.00 | −0.396 | 8.17 | −17.40 |
| ATOM | 3477 | CB | LYS | 542 | 7.869 | −6.388 | −4.323 | 0.00 | 0.00 | −0.106 | 12.77 | 4.00 |
| ATOM | 3478 | HB1 | LYS | 542 | 6.817 | −6.122 | −4.427 | 0.00 | 0.00 | 0.053 | 0.00 | 0.00 |
| ATOM | 3479 | HB2 | LYS | 542 | 7.949 | −7.408 | −4.699 | 0.00 | 0.00 | 0.053 | 0.00 | 0.00 |
| ATOM | 3480 | CG | LYS | 542 | 8.611 | −5.520 | −5.337 | 0.00 | 0.00 | −0.106 | 12.77 | 4.00 |
| ATOM | 3481 | HG1 | LYS | 542 | 9.682 | −5.649 | −5.181 | 0.00 | 0.00 | 0.053 | 0.00 | 0.00 |
| ATOM | 3482 | HG2 | LYS | 542 | 8.323 | −4.481 | −5.175 | 0.00 | 0.00 | 0.053 | 0.00 | 0.00 |
| ATOM | 3483 | CD | LYS | 542 | 8.308 | −5.866 | −6.795 | 0.00 | 0.00 | −0.106 | 12.77 | 4.00 |
| ATOM | 3484 | HD1 | LYS | 542 | 8.213 | −6.937 | −6.973 | 0.00 | 0.00 | 0.053 | 0.00 | 0.00 |
| ATOM | 3485 | HD2 | LYS | 542 | 9.077 | −5.525 | −7.488 | 0.00 | 0.00 | 0.053 | 0.00 | 0.00 |
| ATOM | 3486 | CE | LYS | 542 | 7.004 | −5.253 | −7.303 | 0.00 | 0.00 | 0.099 | 12.77 | 4.00 |
| ATOM | 3487 | HE1 | LYS | 542 | 6.162 | −5.644 | −6.732 | 0.00 | 0.00 | 0.053 | 0.00 | 0.00 |
| ATOM | 3488 | HE2 | LYS | 542 | 6.863 | −5.498 | −8.356 | 0.00 | 0.00 | 0.053 | 0.00 | 0.00 |
| ATOM | 3489 | NZ | LYS | 542 | 7.045 | −3.782 | −7.153 | 0.00 | 0.00 | −0.045 | 13.25 | −39.20 |
| ATOM | 3490 | HZ1 | LYS | 542 | 6.163 | −3.377 | −7.497 | 0.00 | 0.00 | 0.280 | 0.00 | 0.00 |
| ATOM | 3491 | HZ2 | LYS | 542 | 7.168 | −3.541 | −6.159 | 0.00 | 0.00 | 0.280 | 0.00 | 0.00 |
| ATOM | 3492 | HZ3 | LYS | 542 | 7.833 | −3.403 | −7.698 | 0.00 | 0.00 | 0.280 | 0.00 | 0.00 |
| ATOM | 3493 | N | SER | 543 | 7.078 | −4.317 | −2.154 | 0.00 | 0.00 | −0.650 | 9.00 | −17.40 |
| ATOM | 3494 | HN | SER | 543 | 6.306 | −4.979 | −1.988 | 0.00 | 0.00 | 0.439 | 0.00 | 0.00 |
| ATOM | 3495 | CA | SER | 543 | 6.864 | −2.910 | −1.963 | 0.00 | 0.00 | 0.158 | 9.40 | 4.00 |
| ATOM | 3496 | HA | SER | 543 | 7.066 | −2.362 | −2.883 | 0.00 | 0.00 | 0.053 | 0.00 | 0.00 |
| ATOM | 3497 | C | SER | 543 | 7.768 | −2.378 | −0.892 | 0.00 | 0.00 | 0.396 | 9.82 | 4.00 |
| ATOM | 3498 | O | SER | 543 | 8.414 | −1.348 | −1.075 | 0.00 | 0.00 | −0.396 | 8.17 | −17.40 |
| ATOM | 3499 | CB | SER | 543 | 5.422 | −2.581 | −1.538 | 0.00 | 0.00 | 0.007 | 12.77 | 4.00 |
| ATOM | 3500 | HB1 | SER | 543 | 5.187 | −3.076 | −0.596 | 0.00 | 0.00 | 0.053 | 0.00 | 0.00 |
| ATOM | 3501 | HB2 | SER | 543 | 4.720 | −2.921 | −2.299 | 0.00 | 0.00 | 0.053 | 0.00 | 0.00 |
| ATOM | 3502 | OG | SER | 543 | 5.271 | −1.179 | −1.369 | 0.00 | 0.00 | −0.537 | 11.04 | −17.40 |
| ATOM | 3503 | HG | SER | 543 | 6.193 | −0.724 | −1.431 | 0.00 | 0.00 | 0.424 | 0.00 | 0.00 |
| ATOM | 3504 | N | SER | 544 | 7.844 | −3.074 | 0.256 | 0.00 | 0.00 | −0.650 | 9.00 | −17.40 |
| ATOM | 3505 | HN | SER | 544 | 7.335 | −3.966 | 0.340 | 0.00 | 0.00 | 0.439 | 0.00 | 0.00 |
| ATOM | 3506 | CA | SER | 544 | 8.619 | −2.600 | 1.367 | 0.00 | 0.00 | 0.158 | 9.40 | 4.00 |
| ATOM | 3507 | HA | SER | 544 | 8.314 | −1.587 | 1.627 | 0.00 | 0.00 | 0.053 | 0.00 | 0.00 |
| ATOM | 3508 | C | SER | 544 | 10.071 | −2.595 | 1.010 | 0.00 | 0.00 | 0.396 | 9.82 | 4.00 |
| ATOM | 3509 | O | SER | 544 | 10.810 | −1.698 | 1.409 | 0.00 | 0.00 | −0.396 | 8.17 | −17.40 |
| ATOM | 3510 | CB | SER | 544 | 8.455 | −3.475 | 2.622 | 0.00 | 0.00 | 0.007 | 12.77 | 4.00 |
| ATOM | 3511 | HB1 | SER | 544 | 7.401 | −3.560 | 2.886 | 0.00 | 0.00 | 0.053 | 0.00 | 0.00 |
| ATOM | 3512 | HB2 | SER | 544 | 8.992 | −3.033 | 3.461 | 0.00 | 0.00 | 0.053 | 0.00 | 0.00 |
| ATOM | 3513 | OG | SER | 544 | 8.972 | −4.776 | 2.383 | 0.00 | 0.00 | −0.537 | 11.04 | −17.40 |
| ATOM | 3514 | HG | SER | 544 | 8.208 | −5.401 | 2.090 | 0.00 | 0.00 | 0.424 | 0.00 | 0.00 |
| ATOM | 3515 | N | LYS | 545 | 10.522 | −3.606 | 0.246 | 0.00 | 0.00 | −0.650 | 9.00 | −17.40 |
| ATOM | 3516 | HN | LYS | 545 | 9.865 | −4.312 | −0.115 | 0.00 | 0.00 | 0.439 | 0.00 | 0.00 |
| ATOM | 3517 | CA | LYS | 545 | 11.917 | −3.700 | −0.066 | 0.00 | 0.00 | 0.158 | 9.40 | 4.00 |
| ATOM | 3518 | HA | LYS | 545 | 12.490 | −3.726 | 0.861 | 0.00 | 0.00 | 0.053 | 0.00 | 0.00 |
| ATOM | 3519 | C | LYS | 545 | 12.321 | −2.509 | −0.877 | 0.00 | 0.00 | 0.396 | 9.82 | 4.00 |
| ATOM | 3520 | O | LYS | 545 | 13.400 | −1.954 | −0.681 | 0.00 | 0.00 | −0.396 | 8.17 | −17.40 |
| ATOM | 3521 | CB | LYS | 545 | 12.290 | −4.940 | −0.898 | 0.00 | 0.00 | −0.106 | 12.77 | 4.00 |
| ATOM | 3522 | HB1 | LYS | 545 | 11.766 | −5.845 | −0.590 | 0.00 | 0.00 | 0.053 | 0.00 | 0.00 |
| ATOM | 3523 | HB2 | LYS | 545 | 13.349 | −5.195 | −0.850 | 0.00 | 0.00 | 0.053 | 0.00 | 0.00 |
| ATOM | 3524 | CG | LYS | 545 | 11.984 | −4.791 | −2.390 | 0.00 | 0.00 | −0.106 | 12.77 | 4.00 |
| ATOM | 3525 | HG1 | LYS | 545 | 12.259 | −3.816 | −2.792 | 0.00 | 0.00 | 0.053 | 0.00 | 0.00 |
| ATOM | 3526 | HG2 | LYS | 545 | 10.928 | −4.914 | −2.629 | 0.00 | 0.00 | 0.053 | 0.00 | 0.00 |
| ATOM | 3527 | CD | LYS | 545 | 12.716 | −5.809 | −3.263 | 0.00 | 0.00 | −0.106 | 12.77 | 4.00 |
| ATOM | 3528 | HD1 | LYS | 545 | 12.166 | −6.750 | −3.229 | 0.00 | 0.00 | 0.053 | 0.00 | 0.00 |
| ATOM | 3529 | HD2 | LYS | 545 | 13.723 | −5.940 | −2.867 | 0.00 | 0.00 | 0.053 | 0.00 | 0.00 |
| ATOM | 3530 | CE | LYS | 545 | 12.840 | −5.392 | −4.730 | 0.00 | 0.00 | 0.099 | 12.77 | 4.00 |
| ATOM | 3531 | HE1 | LYS | 545 | 13.396 | −6.141 | −5.293 | 0.00 | 0.00 | 0.053 | 0.00 | 0.00 |
| ATOM | 3532 | HE2 | LYS | 545 | 13.363 | −4.439 | −4.812 | 0.00 | 0.00 | 0.053 | 0.00 | 0.00 |
| ATOM | 3533 | NZ | LYS | 545 | 11.503 | −5.246 | −5.339 | 0.00 | 0.00 | −0.045 | 13.25 | −39.20 |
| ATOM | 3534 | HZ1 | LYS | 545 | 11.603 | −4.965 | −6.325 | 0.00 | 0.00 | 0.280 | 0.00 | 0.00 |
| ATOM | 3535 | HZ2 | LYS | 545 | 11.002 | −6.144 | −5.287 | 0.00 | 0.00 | 0.280 | 0.00 | 0.00 |
| ATOM | 3536 | HZ3 | LYS | 545 | 10.970 | −4.526 | −4.830 | 0.00 | 0.00 | 0.280 | 0.00 | 0.00 |
| ATOM | 3537 | N | ASN | 546 | 11.445 | −2.085 | −1.806 | 0.00 | 0.00 | −0.650 | 9.00 | −17.40 |
| ATOM | 3538 | HN | ASN | 546 | 10.536 | −2.565 | −1.874 | 0.00 | 0.00 | 0.439 | 0.00 | 0.00 |
| ATOM | 3539 | CA | ASN | 546 | 11.709 | −1.000 | −2.708 | 0.00 | 0.00 | 0.158 | 9.40 | 4.00 |
| ATOM | 3540 | HA | ASN | 546 | 12.623 | −1.215 | −3.261 | 0.00 | 0.00 | 0.053 | 0.00 | 0.00 |
| ATOM | 3541 | C | ASN | 546 | 11.873 | 0.260 | −1.917 | 0.00 | 0.00 | 0.396 | 9.82 | 4.00 |
| ATOM | 3542 | O | ASN | 546 | 12.584 | 1.177 | −2.326 | 0.00 | 0.00 | −0.396 | 8.17 | −17.40 |
| ATOM | 3543 | CB | ASN | 546 | 10.560 | −0.762 | −3.703 | 0.00 | 0.00 | −0.106 | 12.77 | 4.00 |
| ATOM | 3544 | HB1 | ASN | 546 | 10.781 | 0.146 | −4.265 | 0.00 | 0.00 | 0.053 | 0.00 | 0.00 |
| ATOM | 3545 | HB2 | ASN | 546 | 9.637 | −0.650 | −3.134 | 0.00 | 0.00 | 0.053 | 0.00 | 0.00 |
| ATOM | 3546 | CG | ASN | 546 | 10.481 | −1.965 | −4.630 | 0.00 | 0.00 | 0.396 | 9.82 | 4.00 |
| ATOM | 3547 | OD1 | ASN | 546 | 11.491 | −2.427 | −5.160 | 0.00 | 0.00 | −0.396 | 8.17 | −17.40 |
| ATOM | 3548 | ND2 | ASN | 546 | 9.244 | −2.497 | −4.826 | 0.00 | 0.00 | −0.879 | 13.25 | −17.40 |
| ATOM | 3549 | HD21 | ASN | 546 | 8.424 | −2.079 | −4.363 | 0.00 | 0.00 | 0.439 | 0.00 | 0.00 |
| ATOM | 3550 | HD22 | ASN | 546 | 9.127 | −3.317 | −5.438 | 0.00 | 0.00 | 0.439 | 0.00 | 0.00 |
| ATOM | 3551 | N | THR | 547 | 11.192 | 0.338 | −0.765 | 0.00 | 0.00 | −0.650 | 9.00 | −17.40 |
| ATOM | 3552 | HN | THR | 547 | 10.625 | −0.468 | −0.467 | 0.00 | 0.00 | 0.439 | 0.00 | 0.00 |
| ATOM | 3553 | CA | THR | 547 | 11.226 | 1.504 | 0.065 | 0.00 | 0.00 | 0.158 | 9.40 | 4.00 |
| ATOM | 3554 | HA | THR | 547 | 10.870 | 2.369 | −0.495 | 0.00 | 0.00 | 0.053 | 0.00 | 0.00 |
| ATOM | 3555 | C | THR | 547 | 12.629 | 1.763 | 0.524 | 0.00 | 0.00 | 0.396 | 9.82 | 4.00 |
| ATOM | 3556 | O | THR | 547 | 12.991 | 2.916 | 0.752 | 0.00 | 0.00 | −0.396 | 8.17 | −17.40 |
| ATOM | 3557 | CB | THR | 547 | 10.379 | 1.364 | 1.296 | 0.00 | 0.00 | 0.060 | 9.40 | 4.00 |
| ATOM | 3558 | HB | THR | 547 | 10.739 | 0.511 | 1.871 | 0.00 | 0.00 | 0.053 | 0.00 | 0.00 |
| ATOM | 3559 | OG1 | THR | 547 | 9.025 | 1.136 | 0.938 | 0.00 | 0.00 | −0.537 | 11.04 | −17.40 |
| ATOM | 3560 | HG1 | THR | 547 | 8.541 | 2.039 | 0.831 | 0.00 | 0.00 | 0.424 | 0.00 | 0.00 |
| ATOM | 3561 | CG2 | THR | 547 | 10.499 | 2.655 | 2.125 | 0.00 | 0.00 | −0.159 | 16.15 | 4.00 |
| ATOM | 3562 | HG21 | THR | 547 | 9.888 | 2.568 | 3.024 | 0.00 | 0.00 | 0.053 | 0.00 | 0.00 |
| ATOM | 3563 | HG22 | THR | 547 | 11.540 | 2.811 | 2.407 | 0.00 | 0.00 | 0.053 | 0.00 | 0.00 |
| ATOM | 3564 | HG23 | THR | 547 | 10.154 | 3.502 | 1.532 | 0.00 | 0.00 | 0.053 | 0.00 | 0.00 |
| ATOM | 3565 | N | ASP | 548 | 13.466 | 0.715 | 0.679 | 0.00 | 0.00 | −0.650 | 9.00 | −17.40 |
| ATOM | 3566 | HN | ASP | 548 | 13.166 | −0.238 | 0.428 | 0.00 | 0.00 | 0.439 | 0.00 | 0.00 |
| ATOM | 3567 | CA | ASP | 548 | 14.786 | 0.956 | 1.202 | 0.00 | 0.00 | 0.158 | 9.40 | 4.00 |
| ATOM | 3568 | HA | ASP | 548 | 14.711 | 1.386 | 2.201 | 0.00 | 0.00 | 0.053 | 0.00 | 0.00 |
| ATOM | 3569 | C | ASP | 548 | 15.516 | 1.909 | 0.306 | 0.00 | 0.00 | 0.396 | 9.82 | 4.00 |
| ATOM | 3570 | O | ASP | 548 | 15.231 | 2.023 | −0.886 | 0.00 | 0.00 | −0.396 | 8.17 | −17.40 |
| ATOM | 3571 | CB | ASP | 548 | 15.671 | −0.296 | 1.377 | 0.00 | 0.00 | −0.336 | 12.77 | 4.00 |
| ATOM | 3572 | HB1 | ASP | 548 | 15.131 | −1.076 | 1.913 | 0.00 | 0.00 | 0.053 | 0.00 | 0.00 |
| ATOM | 3573 | HB2 | ASP | 548 | 16.572 | −0.054 | 1.941 | 0.00 | 0.00 | 0.053 | 0.00 | 0.00 |
| ATOM | 3574 | CG | ASP | 548 | 16.090 | −0.850 | 0.022 | 0.00 | 0.00 | 0.297 | 9.82 | 4.00 |
| ATOM | 3575 | OD1 | ASP | 548 | 15.453 | −0.502 | −1.007 | 0.00 | 0.00 | −0.534 | 8.17 | −18.95 |
| ATOM | 3576 | OD2 | ASP | 548 | 17.080 | −1.629 | 0.004 | 0.00 | 0.00 | −0.534 | 8.17 | −18.95 |
| ATOM | 3577 | N | PHE | 549 | 16.485 | 2.644 | 0.889 | 0.00 | 0.00 | −0.650 | 9.00 | −17.40 |
| ATOM | 3578 | HN | PHE | 549 | 16.710 | 2.490 | 1.882 | 0.00 | 0.00 | 0.439 | 0.00 | 0.00 |
| ATOM | 3579 | CA | PHE | 549 | 17.207 | 3.636 | 0.147 | 0.00 | 0.00 | 0.158 | 9.40 | 4.00 |
| ATOM | 3580 | HA | PHE | 549 | 16.510 | 4.289 | −0.378 | 0.00 | 0.00 | 0.053 | 0.00 | 0.00 |
| ATOM | 3581 | C | PHE | 549 | 18.099 | 2.977 | −0.851 | 0.00 | 0.00 | 0.396 | 9.82 | 4.00 |
| ATOM | 3582 | O | PHE | 549 | 18.343 | 3.543 | −1.918 | 0.00 | 0.00 | −0.396 | 8.17 | −17.40 |
| ATOM | 3583 | CB | PHE | 549 | 18.064 | 4.573 | 1.017 | 0.00 | 0.00 | −0.106 | 12.77 | 4.00 |
| ATOM | 3584 | HB1 | PHE | 549 | 17.452 | 5.004 | 1.810 | 0.00 | 0.00 | 0.053 | 0.00 | 0.00 |
| ATOM | 3585 | HB2 | PHE | 549 | 18.472 | 5.377 | 0.404 | 0.00 | 0.00 | 0.053 | 0.00 | 0.00 |
| ATOM | 3586 | CG | PHE | 549 | 19.186 | 3.809 | 1.628 | 0.00 | 0.00 | 0.000 | 7.26 | 0.60 |
| ATOM | 3587 | CD1 | PHE | 549 | 19.011 | 3.104 | 2.794 | 0.00 | 0.00 | −0.127 | 10.80 | 0.60 |
| ATOM | 3588 | HD1 | PHE | 549 | 18.035 | 3.096 | 3.280 | 0.00 | 0.00 | 0.127 | 0.00 | 0.00 |
| ATOM | 3589 | CD2 | PHE | 549 | 20.425 | 3.814 | 1.027 | 0.00 | 0.00 | −0.127 | 10.80 | 0.60 |
| ATOM | 3590 | HD2 | PHE | 549 | 20.574 | 4.373 | 0.103 | 0.00 | 0.00 | 0.127 | 0.00 | 0.00 |
| ATOM | 3591 | CE1 | PHE | 549 | 20.057 | 2.409 | 3.353 | 0.00 | 0.00 | −0.127 | 10.80 | 0.60 |
| ATOM | 3592 | HE1 | PHE | 549 | 19.910 | 1.852 | 4.278 | 0.00 | 0.00 | 0.127 | 0.00 | 0.00 |
| ATOM | 3593 | CE2 | PHE | 549 | 21.475 | 3.121 | 1.580 | 0.00 | 0.00 | −0.127 | 10.80 | 0.60 |
| ATOM | 3594 | HE2 | PHE | 549 | 22.452 | 3.130 | 1.096 | 0.00 | 0.00 | 0.127 | 0.00 | 0.00 |
| ATOM | 3595 | CZ | PHE | 549 | 21.289 | 2.418 | 2.744 | 0.00 | 0.00 | −0.127 | 10.80 | 0.60 |
| ATOM | 3596 | HZ | PHE | 549 | 22.119 | 1.866 | 3.186 | 0.00 | 0.00 | 0.127 | 0.00 | 0.00 |
| ATOM | 3597 | N | PHE | 550 | 18.601 | 1.764 | −0.537 | 0.00 | 0.00 | −0.650 | 9.00 | −17.40 |
| ATOM | 3598 | HN | PHE | 550 | 18.323 | 1.304 | 0.342 | 0.00 | 0.00 | 0.439 | 0.00 | 0.00 |
| ATOM | 3599 | CA | PHE | 550 | 19.521 | 1.115 | −1.426 | 0.00 | 0.00 | 0.158 | 9.40 | 4.00 |
| ATOM | 3600 | HA | PHE | 550 | 20.418 | 1.731 | −1.489 | 0.00 | 0.00 | 0.053 | 0.00 | 0.00 |
| ATOM | 3601 | C | PHE | 550 | 18.863 | 0.993 | −2.758 | 0.00 | 0.00 | 0.396 | 9.82 | 4.00 |
| ATOM | 3602 | O | PHE | 550 | 19.429 | 1.374 | −3.781 | 0.00 | 0.00 | −0.396 | 8.17 | −17.40 |
| ATOM | 3603 | CB | PHE | 550 | 19.866 | −0.328 | −1.008 | 0.00 | 0.00 | −0.106 | 12.77 | 4.00 |
| ATOM | 3604 | HB1 | PHE | 550 | 20.434 | −0.830 | −1.791 | 0.00 | 0.00 | 0.053 | 0.00 | 0.00 |
| ATOM | 3605 | HB2 | PHE | 550 | 18.958 | −0.903 | −0.823 | 0.00 | 0.00 | 0.053 | 0.00 | 0.00 |
| ATOM | 3606 | CG | PHE | 550 | 20.684 | −0.334 | 0.238 | 0.00 | 0.00 | 0.000 | 7.26 | 0.60 |
| ATOM | 3607 | CD1 | PHE | 550 | 22.042 | −0.119 | 0.182 | 0.00 | 0.00 | −0.127 | 10.80 | 0.60 |
| ATOM | 3608 | HD1 | PHE | 550 | 22.519 | 0.066 | −0.781 | 0.00 | 0.00 | 0.127 | 0.00 | 0.00 |
| ATOM | 3609 | CD2 | PHE | 550 | 20.100 | −0.577 | 1.461 | 0.00 | 0.00 | −0.127 | 10.80 | 0.60 |
| ATOM | 3610 | HD2 | PHE | 550 | 19.027 | −0.757 | 1.520 | 0.00 | 0.00 | 0.127 | 0.00 | 0.00 |
| ATOM | 3611 | CE1 | PHE | 550 | 22.805 | −0.135 | 1.326 | 0.00 | 0.00 | −0.127 | 10.80 | 0.60 |
| ATOM | 3612 | HE1 | PHE | 550 | 23.879 | 0.041 | 1.267 | 0.00 | 0.00 | 0.127 | 0.00 | 0.00 |
| ATOM | 3613 | CE2 | PHE | 550 | 20.858 | −0.595 | 2.610 | 0.00 | 0.00 | −0.127 | 10.80 | 0.60 |
| ATOM | 3614 | HE2 | PHE | 550 | 20.384 | −0.785 | 3.573 | 0.00 | 0.00 | 0.127 | 0.00 | 0.00 |
| ATOM | 3615 | CZ | PHE | 550 | 22.213 | −0.373 | 2.543 | 0.00 | 0.00 | −0.127 | 10.80 | 0.60 |
| ATOM | 3616 | HZ | PHE | 550 | 22.815 | −0.386 | 3.451 | 0.00 | 0.00 | 0.127 | 0.00 | 0.00 |
| ATOM | 3617 | N | ILE | 551 | 17.644 | 0.437 | −2.781 | 0.00 | 0.00 | −0.650 | 9.00 | −17.40 |
| ATOM | 3618 | HN | ILE | 551 | 17.200 | 0.120 | −1.907 | 0.00 | 0.00 | 0.439 | 0.00 | 0.00 |
| ATOM | 3619 | CA | ILE | 551 | 16.966 | 0.288 | −4.029 | 0.00 | 0.00 | 0.158 | 9.40 | 4.00 |
| ATOM | 3620 | HA | ILE | 551 | 17.634 | −0.101 | −4.797 | 0.00 | 0.00 | 0.053 | 0.00 | 0.00 |
| ATOM | 3621 | C | ILE | 551 | 16.433 | 1.592 | −4.512 | 0.00 | 0.00 | 0.396 | 9.82 | 4.00 |
| ATOM | 3622 | O | ILE | 551 | 16.543 | 1.917 | −5.692 | 0.00 | 0.00 | −0.396 | 8.17 | −17.40 |
| ATOM | 3623 | CB | ILE | 551 | 15.853 | −0.708 | −3.974 | 0.00 | 0.00 | −0.053 | 9.40 | 4.00 |
| ATOM | 3624 | HB | ILE | 551 | 15.291 | −0.551 | −3.053 | 0.00 | 0.00 | 0.053 | 0.00 | 0.00 |
| ATOM | 3625 | CG1 | ILE | 551 | 16.470 | −2.109 | −3.893 | 0.00 | 0.00 | −0.106 | 12.77 | 4.00 |
| ATOM | 3626 | HG11 | ILE | 551 | 17.122 | −2.247 | −4.755 | 0.00 | 0.00 | 0.053 | 0.00 | 0.00 |
| ATOM | 3627 | HG12 | ILE | 551 | 15.662 | −2.841 | −3.903 | 0.00 | 0.00 | 0.053 | 0.00 | 0.00 |
| ATOM | 3628 | CG2 | ILE | 551 | 14.944 | −0.497 | −5.197 | 0.00 | 0.00 | −0.159 | 16.15 | 4.00 |
| ATOM | 3629 | HG21 | ILE | 551 | 14.126 | −1.217 | −5.171 | 0.00 | 0.00 | 0.053 | 0.00 | 0.00 |
| ATOM | 3630 | HG22 | ILE | 551 | 14.338 | 0.514 | −5.178 | 0.00 | 0.00 | 0.053 | 0.00 | 0.00 |
| ATOM | 3631 | HG23 | ILE | 551 | 15.523 | −0.638 | −6.110 | 0.00 | 0.00 | 0.053 | 0.00 | 0.00 |
| ATOM | 3632 | CD1 | ILE | 551 | 17.304 | −2.352 | −2.634 | 0.00 | 0.00 | −0.159 | 16.15 | 4.00 |
| ATOM | 3633 | HD11 | ILE | 551 | 17.705 | −3.365 | −2.653 | 0.00 | 0.00 | 0.053 | 0.00 | 0.00 |
| ATOM | 3634 | HD12 | ILE | 551 | 18.126 | −1.637 | −2.599 | 0.00 | 0.00 | 0.053 | 0.00 | 0.00 |
| ATOM | 3635 | HD13 | ILE | 551 | 16.676 | −2.227 | −1.752 | 0.00 | 0.00 | 0.053 | 0.00 | 0.00 |
| ATOM | 3636 | N | GLY | 552 | 15.859 | 2.385 | −3.596 | 0.00 | 0.00 | −0.650 | 9.00 | −17.40 |
| ATOM | 3637 | HN | GLY | 552 | 15.913 | 2.129 | −2.600 | 0.00 | 0.00 | 0.439 | 0.00 | 0.00 |
| ATOM | 3638 | CA | GLY | 552 | 15.174 | 3.580 | −3.978 | 0.00 | 0.00 | 0.105 | 9.40 | 4.00 |
| ATOM | 3639 | HA1 | GLY | 552 | 14.684 | 4.086 | −3.146 | 0.00 | 0.00 | 0.053 | 0.00 | 0.00 |
| ATOM | 3640 | HA2 | GLY | 552 | 14.387 | 3.414 | −4.714 | 0.00 | 0.00 | 0.053 | 0.00 | 0.00 |
| ATOM | 3641 | C | GLY | 552 | 16.065 | 4.608 | −4.587 | 0.00 | 0.00 | 0.396 | 9.82 | 4.00 |
| ATOM | 3642 | O | GLY | 552 | 15.744 | 5.138 | −5.650 | 0.00 | 0.00 | −0.396 | 8.17 | −17.40 |
| ATOM | 3643 | N | LYS | 553 | 17.224 | 4.899 | −3.966 | 0.00 | 0.00 | −0.650 | 9.00 | −17.40 |
| ATOM | 3644 | HN | LYS | 553 | 17.577 | 4.317 | −3.193 | 0.00 | 0.00 | 0.439 | 0.00 | 0.00 |
| ATOM | 3645 | CA | LYS | 553 | 17.949 | 6.050 | −4.421 | 0.00 | 0.00 | 0.158 | 9.40 | 4.00 |
| ATOM | 3646 | HA | LYS | 553 | 18.792 | 6.121 | −3.734 | 0.00 | 0.00 | 0.053 | 0.00 | 0.00 |
| ATOM | 3647 | C | LYS | 553 | 16.958 | 7.177 | −4.303 | 0.00 | 0.00 | 0.396 | 9.82 | 4.00 |
| ATOM | 3648 | O | LYS | 553 | 16.037 | 7.107 | −3.492 | 0.00 | 0.00 | −0.396 | 8.17 | −17.40 |
| ATOM | 3649 | CB | LYS | 553 | 18.397 | 5.986 | −5.896 | 0.00 | 0.00 | −0.106 | 12.77 | 4.00 |
| ATOM | 3650 | HB1 | LYS | 553 | 18.824 | 6.954 | −6.157 | 0.00 | 0.00 | 0.053 | 0.00 | 0.00 |
| ATOM | 3651 | HB2 | LYS | 553 | 17.518 | 5.769 | −6.503 | 0.00 | 0.00 | 0.053 | 0.00 | 0.00 |
| ATOM | 3652 | CG | LYS | 553 | 19.450 | 4.926 | −6.238 | 0.00 | 0.00 | −0.106 | 12.77 | 4.00 |
| ATOM | 3653 | HG1 | LYS | 553 | 20.201 | 4.923 | −5.448 | 0.00 | 0.00 | 0.053 | 0.00 | 0.00 |
| ATOM | 3654 | HG2 | LYS | 553 | 19.900 | 5.189 | −7.195 | 0.00 | 0.00 | 0.053 | 0.00 | 0.00 |
| ATOM | 3655 | CD | LYS | 553 | 18.903 | 3.504 | −6.360 | 0.00 | 0.00 | −0.106 | 12.77 | 4.00 |
| ATOM | 3656 | HD1 | LYS | 553 | 17.994 | 3.438 | −6.958 | 0.00 | 0.00 | 0.053 | 0.00 | 0.00 |
| ATOM | 3657 | HD2 | LYS | 553 | 18.647 | 3.053 | −5.401 | 0.00 | 0.00 | 0.053 | 0.00 | 0.00 |
| ATOM | 3658 | CE | LYS | 553 | 19.885 | 2.526 | −7.013 | 0.00 | 0.00 | 0.099 | 12.77 | 4.00 |
| ATOM | 3659 | HE1 | LYS | 553 | 19.518 | 1.505 | −6.911 | 0.00 | 0.00 | 0.053 | 0.00 | 0.00 |
| ATOM | 3660 | HE2 | LYS | 553 | 20.860 | 2.600 | −6.532 | 0.00 | 0.00 | 0.053 | 0.00 | 0.00 |
| ATOM | 3661 | NZ | LYS | 553 | 20.033 | 2.842 | −8.452 | 0.00 | 0.00 | −0.045 | 13.25 | −39.20 |
| ATOM | 3662 | HZ1 | LYS | 553 | 20.694 | 2.180 | −8.883 | 0.00 | 0.00 | 0.280 | 0.00 | 0.00 |
| ATOM | 3663 | HZ2 | LYS | 553 | 19.117 | 2.764 | −8.917 | 0.00 | 0.00 | 0.280 | 0.00 | 0.00 |
| ATOM | 3664 | HZ3 | LYS | 553 | 20.390 | 3.802 | −8.558 | 0.00 | 0.00 | 0.280 | 0.00 | 0.00 |
| ATOM | 3665 | N | PRO | 554 | 17.094 | 8.248 | −5.015 | 0.00 | 0.00 | −0.422 | 9.00 | −17.40 |
| ATOM | 3666 | CA | PRO | 554 | 16.026 | 9.202 | −4.911 | 0.00 | 0.00 | 0.158 | 9.40 | 4.00 |
| ATOM | 3667 | HA | PRO | 554 | 15.638 | 9.209 | −3.893 | 0.00 | 0.00 | 0.053 | 0.00 | 0.00 |
| ATOM | 3668 | CD | PRO | 554 | 18.384 | 8.910 | −5.115 | 0.00 | 0.00 | 0.105 | 12.77 | 4.00 |
| ATOM | 3669 | HD1 | PRO | 554 | 18.958 | 8.274 | −5.789 | 0.00 | 0.00 | 0.053 | 0.00 | 0.00 |
| ATOM | 3670 | HD2 | PRO | 554 | 18.755 | 8.926 | −4.090 | 0.00 | 0.00 | 0.053 | 0.00 | 0.00 |
| ATOM | 3671 | C | PRO | 554 | 14.921 | 8.831 | −5.860 | 0.00 | 0.00 | 0.396 | 9.82 | 4.00 |
| ATOM | 3672 | O | PRO | 554 | 15.216 | 8.417 | −6.979 | 0.00 | 0.00 | −0.396 | 8.17 | −17.40 |
| ATOM | 3673 | CB | PRO | 554 | 16.638 | 10.585 | −5.151 | 0.00 | 0.00 | −0.106 | 12.77 | 4.00 |
| ATOM | 3674 | HB1 | PRO | 554 | 16.626 | 11.064 | −4.172 | 0.00 | 0.00 | 0.053 | 0.00 | 0.00 |
| ATOM | 3675 | HB2 | PRO | 554 | 15.977 | 11.054 | −5.880 | 0.00 | 0.00 | 0.053 | 0.00 | 0.00 |
| ATOM | 3676 | CG | PRO | 554 | 18.051 | 10.295 | −5.686 | 0.00 | 0.00 | −0.106 | 12.77 | 4.00 |
| ATOM | 3677 | HG1 | PRO | 554 | 18.759 | 11.050 | −5.344 | 0.00 | 0.00 | 0.053 | 0.00 | 0.00 |
| ATOM | 3678 | HG2 | PRO | 554 | 18.061 | 10.292 | −6.776 | 0.00 | 0.00 | 0.053 | 0.00 | 0.00 |
| ATOM | 3679 | N | THR | 555 | 13.648 | 8.985 | −5.437 | 0.00 | 0.00 | −0.650 | 9.00 | −17.40 |
| ATOM | 3680 | HN | THR | 555 | 13.507 | 9.371 | −4.492 | 0.00 | 0.00 | 0.439 | 0.00 | 0.00 |
| ATOM | 3681 | CA | THR | 555 | 12.463 | 8.652 | −6.202 | 0.00 | 0.00 | 0.158 | 9.40 | 4.00 |
| ATOM | 3682 | HA | THR | 555 | 12.769 | 8.520 | −7.240 | 0.00 | 0.00 | 0.053 | 0.00 | 0.00 |
| ATOM | 3683 | C | THR | 555 | 11.490 | 9.800 | −6.056 | 0.00 | 0.00 | 0.396 | 9.82 | 4.00 |
| ATOM | 3684 | O | THR | 555 | 11.620 | 10.518 | −5.070 | 0.00 | 0.00 | −0.396 | 8.17 | −17.40 |
| ATOM | 3685 | CB | THR | 555 | 11.766 | 7.445 | −5.635 | 0.00 | 0.00 | 0.060 | 9.40 | 4.00 |
| ATOM | 3686 | HB | THR | 555 | 11.604 | 7.569 | −4.564 | 0.00 | 0.00 | 0.053 | 0.00 | 0.00 |
| ATOM | 3687 | OG1 | THR | 555 | 12.589 | 6.294 | −5.764 | 0.00 | 0.00 | −0.537 | 11.04 | −17.40 |
| ATOM | 3688 | HG1 | THR | 555 | 12.002 | 5.472 | −5.965 | 0.00 | 0.00 | 0.424 | 0.00 | 0.00 |
| ATOM | 3689 | CG2 | THR | 555 | 10.403 | 7.246 | −6.316 | 0.00 | 0.00 | −0.159 | 16.15 | 4.00 |
| ATOM | 3690 | HG21 | THR | 555 | 9.911 | 6.369 | −5.895 | 0.00 | 0.00 | 0.053 | 0.00 | 0.00 |
| ATOM | 3691 | HG22 | THR | 555 | 9.781 | 8.126 | −6.150 | 0.00 | 0.00 | 0.053 | 0.00 | 0.00 |
| ATOM | 3692 | HG23 | THR | 555 | 10.549 | 7.102 | −7.387 | 0.00 | 0.00 | 0.053 | 0.00 | 0.00 |
| ATOM | 3693 | N | LEU | 556 | 10.491 | 9.980 | −6.988 | 0.00 | 0.00 | −0.650 | 9.00 | −17.40 |
| ATOM | 3694 | HN | LEU | 556 | 10.371 | 9.267 | −7.721 | 0.00 | 0.00 | 0.439 | 0.00 | 0.00 |
| ATOM | 3695 | CA | LEU | 556 | 9.590 | 11.138 | −6.987 | 0.00 | 0.00 | 0.158 | 9.40 | 4.00 |
| ATOM | 3696 | HA | LEU | 556 | 9.460 | 11.572 | −5.996 | 0.00 | 0.00 | 0.053 | 0.00 | 0.00 |
| ATOM | 3697 | C | LEU | 556 | 8.156 | 10.790 | −7.493 | 0.00 | 0.00 | 0.396 | 9.82 | 4.00 |
| ATOM | 3698 | O | LEU | 556 | 7.980 | 9.709 | −8.050 | 0.00 | 0.00 | −0.396 | 8.17 | −17.40 |
| ATOM | 3699 | CB | LEU | 556 | 10.157 | 12.202 | −7.938 | 0.00 | 0.00 | −0.106 | 12.77 | 4.00 |
| ATOM | 3700 | HB1 | LEU | 556 | 9.658 | 13.165 | −7.824 | 0.00 | 0.00 | 0.053 | 0.00 | 0.00 |
| ATOM | 3701 | HB2 | LEU | 556 | 10.050 | 11.918 | −8.985 | 0.00 | 0.00 | 0.053 | 0.00 | 0.00 |
| ATOM | 3702 | CG | LEU | 556 | 11.663 | 12.455 | −7.701 | 0.00 | 0.00 | −0.053 | 9.40 | 4.00 |
| ATOM | 3703 | HG | LEU | 556 | 12.239 | 11.530 | −7.704 | 0.00 | 0.00 | 0.053 | 0.00 | 0.00 |
| ATOM | 3704 | CD1 | LEU | 556 | 12.273 | 13.360 | −8.782 | 0.00 | 0.00 | −0.159 | 16.15 | 4.00 |
| ATOM | 3705 | HD11 | LEU | 556 | 13.333 | 13.511 | −8.575 | 0.00 | 0.00 | 0.053 | 0.00 | 0.00 |
| ATOM | 3706 | HD12 | LEU | 556 | 12.157 | 12.889 | −9.758 | 0.00 | 0.00 | 0.053 | 0.00 | 0.00 |
| ATOM | 3707 | HD13 | LEU | 556 | 11.763 | 14.323 | −8.781 | 0.00 | 0.00 | 0.053 | 0.00 | 0.00 |
| ATOM | 3708 | CD2 | LEU | 556 | 11.930 | 12.966 | −6.277 | 0.00 | 0.00 | −0.159 | 16.15 | 4.00 |
| ATOM | 3709 | HD21 | LEU | 556 | 12.999 | 13.133 | −6.145 | 0.00 | 0.00 | 0.053 | 0.00 | 0.00 |
| ATOM | 3710 | HD22 | LEU | 556 | 11.394 | 13.902 | −6.120 | 0.00 | 0.00 | 0.053 | 0.00 | 0.00 |
| ATOM | 3711 | HD23 | LEU | 556 | 11.587 | 12.226 | −5.554 | 0.00 | 0.00 | 0.053 | 0.00 | 0.00 |
| ATOM | 3712 | N | ARG | 557 | 7.115 | 11.712 | −7.357 | 0.00 | 0.00 | −0.650 | 9.00 | −17.40 |
| ATOM | 3713 | HN | ARG | 557 | 7.381 | 12.648 | −7.020 | 0.00 | 0.00 | 0.439 | 0.00 | 0.00 |
| ATOM | 3714 | CA | ARG | 557 | 5.669 | 11.506 | −7.639 | 0.00 | 0.00 | 0.158 | 9.40 | 4.00 |
| ATOM | 3715 | HA | ARG | 557 | 5.574 | 10.620 | −8.266 | 0.00 | 0.00 | 0.053 | 0.00 | 0.00 |
| ATOM | 3716 | C | ARG | 557 | 4.954 | 12.652 | −8.369 | 0.00 | 0.00 | 0.396 | 9.82 | 4.00 |
| ATOM | 3717 | O | ARG | 557 | 5.573 | 13.538 | −8.954 | 0.00 | 0.00 | −0.396 | 8.17 | −17.40 |
| ATOM | 3718 | CB | ARG | 557 | 4.778 | 11.470 | −6.393 | 0.00 | 0.00 | −0.106 | 12.77 | 4.00 |
| ATOM | 3719 | HB1 | ARG | 557 | 3.760 | 11.670 | −6.728 | 0.00 | 0.00 | 0.053 | 0.00 | 0.00 |
| ATOM | 3720 | HB2 | ARG | 557 | 5.147 | 12.244 | −5.720 | 0.00 | 0.00 | 0.053 | 0.00 | 0.00 |
| ATOM | 3721 | CG | ARG | 557 | 4.687 | 10.210 | −5.550 | 0.00 | 0.00 | −0.106 | 12.77 | 4.00 |
| ATOM | 3722 | HG1 | ARG | 557 | 5.687 | 9.986 | −5.178 | 0.00 | 0.00 | 0.053 | 0.00 | 0.00 |
| ATOM | 3723 | HG2 | ARG | 557 | 4.317 | 9.408 | −6.189 | 0.00 | 0.00 | 0.053 | 0.00 | 0.00 |
| ATOM | 3724 | CD | ARG | 557 | 3.718 | 10.488 | −4.396 | 0.00 | 0.00 | 0.374 | 12.77 | 4.00 |
| ATOM | 3725 | HD1 | ARG | 557 | 2.728 | 10.718 | −4.789 | 0.00 | 0.00 | 0.053 | 0.00 | 0.00 |
| ATOM | 3726 | HD2 | ARG | 557 | 4.072 | 11.335 | −3.809 | 0.00 | 0.00 | 0.053 | 0.00 | 0.00 |
| ATOM | 3727 | NE | ARG | 557 | 3.623 | 9.296 | −3.516 | 0.00 | 0.00 | −0.819 | 9.00 | −24.67 |
| ATOM | 3728 | HE | ARG | 557 | 3.562 | 8.351 | −3.921 | 0.00 | 0.00 | 0.407 | 0.00 | 0.00 |
| ATOM | 3729 | CZ | ARG | 557 | 3.618 | 9.472 | −2.164 | 0.00 | 0.00 | 0.796 | 6.95 | 4.00 |
| ATOM | 3730 | NH1 | ARG | 557 | 3.862 | 10.712 | −1.647 | 0.00 | 0.00 | −0.746 | 9.00 | −24.67 |
| ATOM | 3731 | HH11 | ARG | 557 | 3.860 | 10.854 | −0.627 | 0.00 | 0.00 | 0.407 | 0.00 | 0.00 |
| ATOM | 3732 | HH12 | ARG | 557 | 4.048 | 11.505 | −2.277 | 0.00 | 0.00 | 0.407 | 0.00 | 0.00 |
| ATOM | 3733 | NH2 | ARG | 557 | 3.374 | 8.416 | −1.333 | 0.00 | 0.00 | −0.746 | 9.00 | −24.67 |
| ATOM | 3734 | HH21 | ARG | 557 | 3.371 | 8.554 | −0.312 | 0.00 | 0.00 | 0.407 | 0.00 | 0.00 |
| ATOM | 3735 | HH22 | ARG | 557 | 3.193 | 7.482 | −1.727 | 0.00 | 0.00 | 0.407 | 0.00 | 0.00 |
| ATOM | 3736 | N | GLU | 558 | 3.571 | 12.607 | −8.333 | 0.00 | 0.00 | −0.650 | 9.00 | −17.40 |
| ATOM | 3737 | HN | GLU | 558 | 3.164 | 11.797 | −7.843 | 0.00 | 0.00 | 0.439 | 0.00 | 0.00 |
| ATOM | 3738 | CA | GLU | 558 | 2.607 | 13.556 | −8.898 | 0.00 | 0.00 | 0.158 | 9.40 | 4.00 |
| ATOM | 3739 | HA | GLU | 558 | 3.101 | 14.487 | −9.177 | 0.00 | 0.00 | 0.053 | 0.00 | 0.00 |
| ATOM | 3740 | C | GLU | 558 | 1.518 | 13.880 | −7.890 | 0.00 | 0.00 | 0.396 | 9.82 | 4.00 |
| ATOM | 3741 | O | GLU | 558 | 1.129 | 13.032 | −7.088 | 0.00 | 0.00 | −0.396 | 8.17 | −17.40 |
| ATOM | 3742 | CB | GLU | 558 | 1.910 | 13.049 | −10.170 | 0.00 | 0.00 | −0.106 | 12.77 | 4.00 |
| ATOM | 3743 | HB1 | GLU | 558 | 0.989 | 13.588 | −10.395 | 0.00 | 0.00 | 0.053 | 0.00 | 0.00 |
| ATOM | 3744 | HB2 | GLU | 558 | 1.630 | 11.997 | −10.112 | 0.00 | 0.00 | 0.053 | 0.00 | 0.00 |
| ATOM | 3745 | CG | GLU | 558 | 2.781 | 13.177 | −11.421 | 0.00 | 0.00 | −0.336 | 12.77 | 4.00 |
| ATOM | 3746 | HG1 | GLU | 558 | 3.025 | 14.230 | −11.564 | 0.00 | 0.00 | 0.053 | 0.00 | 0.00 |
| ATOM | 3747 | HG2 | GLU | 558 | 2.217 | 12.796 | −12.273 | 0.00 | 0.00 | 0.053 | 0.00 | 0.00 |
| ATOM | 3748 | CD | GLU | 558 | 4.046 | 12.360 | −11.210 | − 0.00 | 0.00 | 0.297 | 9.82 | 4.00 |
| ATOM | 3749 | OE1 | GLU | 558 | 3.929 | 11.167 | −10.823 | 0.00 | 0.00 | −0.534 | 8.17 | −18.95 |
| ATOM | 3750 | OE2 | GLU | 558 | 5.152 | 12.925 | −11.428 | 0.00 | 0.00 | −0.534 | 8.17 | −18.95 |
| ATOM | 3751 | N | LEU | 559 | 0.975 | 15.124 | −7.976 | 0.00 | 0.00 | −0.650 | 9.00 | −17.40 |
| ATOM | 3752 | HN | LEU | 559 | 1.243 | 15.663 | −8.812 | 0.00 | 0.00 | 0.439 | 0.00 | 0.00 |
| ATOM | 3753 | CA | LEU | 559 | 0.073 | 15.804 | −7.063 | 0.00 | 0.00 | 0.158 | 9.40 | 4.00 |
| ATOM | 3754 | HA | LEU | 559 | 0.557 | 15.851 | −6.088 | 0.00 | 0.00 | 0.053 | 0.00 | 0.00 |
| ATOM | 3755 | C | LEU | 559 | −1.274 | 15.155 | −6.857 | 0.00 | 0.00 | 0.396 | 9.82 | 4.00 |
| ATOM | 3756 | O | LEU | 559 | −1.655 | 14.915 | −5.711 | 0.00 | 0.00 | −0.396 | 8.17 | −17.40 |
| ATOM | 3757 | CB | LEU | 559 | −0.123 | 17.261 | −7.516 | 0.00 | 0.00 | −0.106 | 12.77 | 4.00 |
| ATOM | 3758 | HB1 | LEU | 559 | −0.550 | 17.237 | −8.518 | 0.00 | 0.00 | 0.053 | 0.00 | 0.00 |
| ATOM | 3759 | HB2 | LEU | 559 | 0.856 | 17.739 | −7.512 | 0.00 | 0.00 | 0.053 | 0.00 | 0.00 |
| ATOM | 3760 | CG | LEU | 559 | −1.045 | 18.151 | −6.671 | 0.00 | 0.00 | −0.053 | 9.40 | 4.00 |
| ATOM | 3761 | HG | LEU | 559 | −2.060 | 17.757 | −6.634 | 0.00 | 0.00 | 0.053 | 0.00 | 0.00 |
| ATOM | 3762 | CD1 | LEU | 559 | −0.548 | 18.266 | −5.221 | 0.00 | 0.00 | −0.159 | 16.15 | 4.00 |
| ATOM | 3763 | HD11 | LEU | 559 | −1.226 | 18.904 | −4.654 | 0.00 | 0.00 | 0.053 | 0.00 | 0.00 |
| ATOM | 3764 | HD12 | LEU | 559 | −0.517 | 17.275 | −4.767 | 0.00 | 0.00 | 0.053 | 0.00 | 0.00 |
| ATOM | 3765 | HD13 | LEU | 559 | 0.452 | 18.701 | −5.212 | 0.00 | 0.00 | 0.053 | 0.00 | 0.00 |
| ATOM | 3766 | CD2 | LEU | 559 | −1.181 | 19.532 | −7.334 | 0.00 | 0.00 | −0.159 | 16.15 | 4.00 |
| ATOM | 3767 | HD21 | LEU | 559 | −1.836 | 20.162 | −6.732 | 0.00 | 0.00 | 0.053 | 0.00 | 0.00 |
| ATOM | 3768 | HD22 | LEU | 559 | −0.198 | 19.998 | −7.409 | 0.00 | 0.00 | 0.053 | 0.00 | 0.00 |
| ATOM | 3769 | HD23 | LEU | 559 | −1.605 | 19.417 | −8.331 | 0.00 | 0.00 | 0.053 | 0.00 | 0.00 |
| ATOM | 3770 | N | THR | 560 | −2.054 | 14.846 | −7.913 | 0.00 | 0.00 | −0.650 | 9.00 | −17.40 |
| ATOM | 3771 | HN | THR | 560 | −1.769 | 15.046 | −8.882 | 0.00 | 0.00 | 0.439 | 0.00 | 0.00 |
| ATOM | 3772 | CA | THR | 560 | −3.313 | 14.218 | −7.597 | 0.00 | 0.00 | 0.158 | 9.40 | 4.00 |
| ATOM | 3773 | HA | THR | 560 | −3.368 | 14.187 | −6.509 | 0.00 | 0.00 | 0.053 | 0.00 | 0.00 |
| ATOM | 3774 | C | THR | 560 | −3.259 | 12.860 | −8.210 | 0.00 | 0.00 | 0.396 | 9.82 | 4.00 |
| ATOM | 3775 | O | THR | 560 | −2.557 | 12.648 | −9.196 | 0.00 | 0.00 | −0.396 | 8.17 | −17.40 |
| ATOM | 3776 | CB | THR | 560 | −4.557 | 14.872 | −8.121 | 0.00 | 0.00 | 0.060 | 9.40 | 4.00 |
| ATOM | 3777 | HB | THR | 560 | −4.417 | 15.952 | −8.077 | 0.00 | 0.00 | 0.053 | 0.00 | 0.00 |
| ATOM | 3778 | OG1 | THR | 560 | −5.678 | 14.448 | −7.359 | 0.00 | 0.00 | −0.537 | 11.04 | −17.40 |
| ATOM | 3779 | HG1 | THR | 560 | −6.239 | 15.268 | −7.088 | 0.00 | 0.00 | 0.424 | 0.00 | 0.00 |
| ATOM | 3780 | CG2 | THR | 560 | −4.766 | 14.409 | −9.568 | 0.00 | 0.00 | −0.159 | 16.15 | 4.00 |
| ATOM | 3781 | HG21 | THR | 560 | −5.666 | 14.872 | −9.972 | 0.00 | 0.00 | 0.053 | 0.00 | 0.00 |
| ATOM | 3782 | HG22 | THR | 560 | −3.906 | 14.701 | −10.171 | 0.00 | 0.00 | 0.053 | 0.00 | 0.00 |
| ATOM | 3783 | HG23 | THR | 560 | −4.874 | 13.325 | −9.591 | 0.00 | 0.00 | 0.053 | 0.00 | 0.00 |
| ATOM | 3784 | N | SER | 561 | −4.012 | 11.905 | −7.632 | 0.00 | 0.00 | −0.650 | 9.00 | −17.40 |
| ATOM | 3785 | HN | SER | 561 | −4.655 | 12.169 | −6.872 | 0.00 | 0.00 | 0.439 | 0.00 | 0.00 |
| ATOM | 3786 | CA | SER | 561 | −3.948 | 10.530 | −8.041 | 0.00 | 0.00 | 0.158 | 9.40 | 4.00 |
| ATOM | 3787 | HA | SER | 561 | −2.930 | 10.142 | −8.075 | 0.00 | 0.00 | 0.053 | 0.00 | 0.00 |
| ATOM | 3788 | C | SER | 561 | −4.514 | 10.298 | −9.405 | 0.00 | 0.00 | 0.396 | 9.82 | 4.00 |
| ATOM | 3789 | O | SER | 561 | −5.589 | 10.786 | −9.746 | 0.00 | 0.00 | −0.396 | 8.17 | −17.40 |
| ATOM | 3790 | CB | SER | 561 | −4.695 | 9.585 | −7.089 | 0.00 | 0.00 | 0.007 | 12.77 | 4.00 |
| ATOM | 3791 | HB1 | SER | 561 | −4.285 | 9.660 | −6.082 | 0.00 | 0.00 | 0.053 | 0.00 | 0.00 |
| ATOM | 3792 | HB2 | SER | 561 | −4.600 | 8.553 | −7.427 | 0.00 | 0.00 | 0.053 | 0.00 | 0.00 |
| ATOM | 3793 | OG | SER | 561 | −6.073 | 9.920 | −7.042 | 0.00 | 0.00 | −0.537 | 11.04 | −17.40 |
| ATOM | 3794 | HG | SER | 561 | −6.178 | 10.945 | −7.034 | 0.00 | 0.00 | 0.424 | 0.00 | 0.00 |
| ATOM | 3795 | N | GLN | 562 | −3.749 | 9.542 | −10.223 | 0.00 | 0.00 | −0.650 | 9.00 | −17.40 |
| ATOM | 3796 | HN | GLN | 562 | −2.824 | 9.254 | −9.873 | 0.00 | 0.00 | 0.439 | 0.00 | 0.00 |
| ATOM | 3797 | CA | GLN | 562 | −4.120 | 9.108 | −11.539 | 0.00 | 0.00 | 0.158 | 9.40 | 4.00 |
| ATOM | 3798 | HA | GLN | 562 | −4.546 | 9.939 | −12.101 | 0.00 | 0.00 | 0.053 | 0.00 | 0.00 |
| ATOM | 3799 | C | GLN | 562 | −5.133 | 8.015 | −11.452 | 0.00 | 0.00 | 0.396 | 9.82 | 4.00 |
| ATOM | 3800 | O | GLN | 562 | −5.975 | 7.875 | −12.335 | 0.00 | 0.00 | −0.396 | 8.17 | −17.40 |
| ATOM | 3801 | CB | GLN | 562 | −2.945 | 8.579 | −12.384 | 0.00 | 0.00 | −0.106 | 12.77 | 4.00 |
| ATOM | 3802 | HB1 | GLN | 562 | −3.339 | 7.861 | −13.103 | 0.00 | 0.00 | 0.053 | 0.00 | 0.00 |
| ATOM | 3803 | HB2 | GLN | 562 | −2.232 | 8.101 | −11.713 | 0.00 | 0.00 | 0.053 | 0.00 | 0.00 |
| ATOM | 3804 | CG | GLN | 562 | −2.187 | 9.652 | −13.173 | 0.00 | 0.00 | −0.106 | 12.77 | 4.00 |
| ATOM | 3805 | HG1 | GLN | 562 | −2.869 | 10.290 | −13.735 | 0.00 | 0.00 | 0.053 | 0.00 | 0.00 |
| ATOM | 3806 | HG2 | GLN | 562 | −1.494 | 9.206 | −13.886 | 0.00 | 0.00 | 0.053 | 0.00 | 0.00 |
| ATOM | 3807 | CD | GLN | 562 | −1.378 | 10.551 | −12.252 | 0.00 | 0.00 | 0.396 | 9.82 | 4.00 |
| ATOM | 3808 | OE1 | GLN | 562 | −1.857 | 11.041 | −11.231 | 0.00 | 0.00 | −0.396 | 8.17 | −17.40 |
| ATOM | 3809 | NE2 | GLN | 562 | −0.100 | 10.799 | −12.645 | 0.00 | 0.00 | −0.879 | 13.25 | −17.40 |
| ATOM | 3810 | HE21 | GLN | 562 | 0.266 | 10.370 | −13.507 | 0.00 | 0.00 | 0.439 | 0.00 | 0.00 |
| ATOM | 3811 | HE22 | GLN | 562 | 0.502 | 11.416 | −12.081 | 0.00 | 0.00 | 0.439 | 0.00 | 0.00 |
| ATOM | 3812 | N | HIS | 563 | −5.070 | 7.151 | −10.424 | 0.00 | 0.00 | −0.650 | 9.00 | −17.40 |
| ATOM | 3813 | HN | HIS | 563 | −4.348 | 7.194 | −9.690 | 0.00 | 0.00 | 0.439 | 0.00 | 0.00 |
| ATOM | 3814 | CA | HIS | 563 | −6.103 | 6.170 | −10.452 | 0.00 | 0.00 | 0.158 | 9.40 | 4.00 |
| ATOM | 3815 | HA | HIS | 563 | −7.011 | 6.512 | −10.948 | 0.00 | 0.00 | 0.053 | 0.00 | 0.00 |
| ATOM | 3816 | C | HIS | 563 | −6.538 | 5.730 | −9.096 | 0.00 | 0.00 | 0.396 | 9.82 | 4.00 |
| ATOM | 3817 | O | HIS | 563 | −5.947 | 6.012 | −8.054 | 0.00 | 0.00 | −0.396 | 8.17 | −17.40 |
| ATOM | 3818 | CB | HIS | 563 | −5.882 | 4.993 | −11.416 | 0.00 | 0.00 | −0.106 | 12.77 | 4.00 |
| ATOM | 3819 | HB1 | HIS | 563 | −6.052 | 5.373 | −12.423 | 0.00 | 0.00 | 0.053 | 0.00 | 0.00 |
| ATOM | 3820 | HB2 | HIS | 563 | −6.603 | 4.222 | −11.145 | 0.00 | 0.00 | 0.053 | 0.00 | 0.00 |
| ATOM | 3821 | CG | HIS | 563 | −4.558 | 4.306 | −11.463 | 0.00 | 0.00 | 0.241 | 7.26 | 0.60 |
| ATOM | 3822 | ND1 | HIS | 563 | −4.112 | 3.392 | −10.539 | 0.00 | 0.00 | −0.481 | 9.25 | −17.40 |
| ATOM | 3823 | CD2 | HIS | 563 | −3.601 | 4.362 | −12.430 | 0.00 | 0.00 | −0.177 | 10.80 | 0.60 |
| ATOM | 3824 | HD2 | HIS | 563 | −3.646 | 5.000 | −13.313 | 0.00 | 0.00 | 0.127 | 0.00 | 0.00 |
| ATOM | 3825 | CE1 | HIS | 563 | −2.919 | 2.938 | −10.992 | 0.00 | 0.00 | 0.064 | 10.80 | 0.60 |
| ATOM | 3826 | HE1 | HIS | 563 | −2.317 | 2.193 | −10.471 | 0.00 | 0.00 | 0.127 | 0.00 | 0.00 |
| ATOM | 3827 | NE2 | HIS | 563 | −2.565 | 3.497 | −12.135 | 0.00 | 0.00 | −0.292 | 9.25 | −17.40 |
| ATOM | 3828 | HE2 | HIS | 563 | −1.710 | 3.327 | −12.683 | 0.00 | 0.00 | 0.393 | 0.00 | 0.00 |
| ATOM | 3829 | N | PRO | 564 | −7.690 | 5.120 | −9.185 | 0.00 | 0.00 | −0.422 | 9.00 | −17.40 |
| ATOM | 3830 | CA | PRO | 564 | −8.398 | 4.641 | −8.031 | 0.00 | 0.00 | 0.158 | 9.40 | 4.00 |
| ATOM | 3331 | HA | PRO | 564 | −8.388 | 5.436 | −7.285 | 0.00 | 0.00 | 0.053 | 0.00 | 0.00 |
| ATOM | 3832 | CD | PRO | 564 | −8.610 | 5.546 | −10.231 | 0.00 | 0.00 | 0.105 | 12.77 | 4.00 |
| ATOM | 3833 | HD1 | PRO | 564 | −8.461 | 4.859 | −11.064 | 0.00 | 0.00 | 0.053 | 0.00 | 0.00 |
| ATOM | 3834 | HD2 | PRO | 564 | −8.334 | 6.571 | −10.479 | 0.00 | 0.00 | 0.053 | 0.00 | 0.00 |
| ATOM | 3835 | C | PRO | 564 | −7.863 | 3.424 | −7.363 | 0.00 | 0.00 | 0.396 | 9.82 | 4.00 |
| ATOM | 3836 | O | PRO | 564 | −7.559 | 2.442 | −8.036 | 0.00 | 0.00 | −0.396 | 8.17 | −17.40 |
| ATOM | 3837 | CB | PRO | 564 | −9.849 | 4.447 | −8.475 | 0.00 | 0.00 | −0.106 | 12.77 | 4.00 |
| ATOM | 3838 | HB1 | PRO | 564 | −10.538 | 4.660 | −7.657 | 0.00 | 0.00 | 0.053 | 0.00 | 0.00 |
| ATOM | 3839 | HB2 | PRO | 564 | −10.022 | 3.422 | −8.803 | 0.00 | 0.00 | 0.053 | 0.00 | 0.00 |
| ATOM | 3840 | CG | PRO | 564 | −10.017 | 5.445 | −9.629 | 0.00 | 0.00 | −0.106 | 12.77 | 4.00 |
| ATOM | 3841 | HG1 | PRO | 564 | −10.367 | 6.410 | −9.262 | 0.00 | 0.00 | 0.053 | 0.00 | 0.00 |
| ATOM | 3842 | HG2 | PRO | 564 | −10.741 | 5.080 | −10.358 | 0.00 | 0.00 | 0.053 | 0.00 | 0.00 |
| ATOM | 3843 | N | TYR | 565 | −7.782 | 3.478 | −6.024 | 0.00 | 0.00 | −0.650 | 9.00 | −17.40 |
| ATOM | 3844 | HN | TYR | 565 | −8.016 | 4.368 | −5.562 | 0.00 | 0.00 | 0.439 | 0.00 | 0.00 |
| ATOM | 3845 | CA | TYR | 565 | −7.391 | 2.378 | −5.198 | 0.00 | 0.00 | 0.158 | 9.40 | 4.00 |
| ATOM | 3846 | HA | TYR | 565 | −7.752 | 1.433 | −5.604 | 0.00 | 0.00 | 0.053 | 0.00 | 0.00 |
| ATOM | 3847 | C | TYR | 565 | −8.026 | 2.701 | −3.891 | 0.00 | 0.00 | 0.396 | 9.82 | 4.00 |
| ATOM | 3848 | O | TYR | 565 | −7.533 | 3.554 | −3.159 | 0.00 | 0.00 | −0.396 | 8.17 | −17.40 |
| ATOM | 3849 | CB | TYR | 565 | −5.863 | 2.355 | −4.992 | 0.00 | 0.00 | −0.106 | 12.77 | 4.00 |
| ATOM | 3850 | HB1 | TYR | 565 | −5.492 | 3.289 | −4.570 | 0.00 | 0.00 | 0.053 | 0.00 | 0.00 |
| ATOM | 3851 | HB2 | TYR | 565 | −5.326 | 2.198 | −5.927 | 0.00 | 0.00 | 0.053 | 0.00 | 0.00 |
| ATOM | 3852 | CG | TYR | 565 | −5.439 | 1.267 | −4.065 | 0.00 | 0.00 | 0.000 | 7.26 | 0.60 |
| ATOM | 3853 | CD1 | TYR | 565 | −5.442 | 1.466 | −2.704 | 0.00 | 0.00 | −0.127 | 10.80 | 0.60 |
| ATOM | 3854 | HD1 | TYR | 565 | −5.768 | 2.424 | −2.299 | 0.00 | 0.00 | 0.127 | 0.00 | 0.00 |
| ATOM | 3855 | CD2 | TYR | 565 | −5.011 | 0.053 | −4.552 | 0.00 | 0.00 | −0.127 | 10.80 | 0.60 |
| ATOM | 3856 | HD2 | TYR | 565 | −4.991 | −0.116 | −5.629 | 0.00 | 0.00 | 0.127 | 0.00 | 0.00 |
| ATOM | 3857 | CE1 | TYR | 565 | −5.038 | 0.467 | −1.849 | 0.00 | 0.00 | −0.127 | 10.80 | 0.60 |
| ATOM | 3858 | HE1 | TYR | 565 | −5.050 | 0.637 | −0.772 | 0.00 | 0.00 | 0.127 | 0.00 | 0.00 |
| ATOM | 3859 | CE2 | TYR | 565 | −4.608 | −0.950 | −3.703 | 0.00 | 0.00 | −0.127 | 10.80 | 0.60 |
| ATOM | 3860 | HE2 | TYR | 565 | −4.280 | −1.908 | −4.106 | 0.00 | 0.00 | 0.127 | 0.00 | 0.00 |
| ATOM | 3861 | CZ | TYR | 565 | −4.620 | −0.743 | −2.345 | 0.00 | 0.00 | 0.027 | 7.26 | 0.60 |
| ATOM | 3862 | OH | TYR | 565 | −4.206 | −1.767 | −1.467 | 0.00 | 0.00 | −0.451 | 10.94 | −17.40 |
| ATOM | 3863 | HH | TYR | 565 | −4.810 | −1.769 | −0.633 | 0.00 | 0.00 | 0.424 | 0.00 | 0.00 |
| ATOM | 3864 | N | ALA | 566 | −9.142 | 2.038 | −3.551 | 0.00 | 0.00 | −0.650 | 9.00 | −17.40 |
| ATOM | 3865 | HN | ALA | 566 | −9.536 | 1.300 | −4.152 | 0.00 | 0.00 | 0.439 | 0.00 | 0.00 |
| ATOM | 3866 | CA | ALA | 566 | −9.758 | 2.405 | −2.315 | 0.00 | 0.00 | 0.158 | 9.40 | 4.00 |
| ATOM | 3867 | HA | ALA | 566 | −8.962 | 2.661 | −1.616 | 0.00 | 0.00 | 0.053 | 0.00 | 0.00 |
| ATOM | 3868 | C | ALA | 566 | −10.539 | 1.235 | −1.842 | 0.00 | 0.00 | 0.396 | 9.82 | 4.00 |
| ATOM | 3869 | O | ALA | 566 | −10.422 | 0.152 | −2.404 | 0.00 | 0.00 | −0.396 | 8.17 | −17.40 |
| ATOM | 3870 | CB | ALA | 566 | −10.716 | 3.591 | −2.447 | 0.00 | 0.00 | −0.159 | 16.15 | 4.00 |
| ATOM | 3871 | HB1 | ALA | 566 | −11.150 | 3.818 | −1.473 | 0.00 | 0.00 | 0.053 | 0.00 | 0.00 |
| ATOM | 3872 | HB2 | ALA | 566 | −10.170 | 4.461 | −2.813 | 0.00 | 0.00 | 0.053 | 0.00 | 0.00 |
| ATOM | 3873 | HB3 | ALA | 566 | −11.511 | 3.340 | −3.149 | 0.00 | 0.00 | 0.053 | 0.00 | 0.00 |
| ATOM | 3874 | N | GLU | 567 | −11.395 | 1.448 | −0.826 | 0.00 | 0.00 | −0.650 | 9.00 | −17.40 |
| ATOM | 3875 | HN | GLU | 567 | −11.544 | 2.415 | −0.504 | 0.00 | 0.00 | 0.439 | 0.00 | 0.00 |
| ATOM | 3876 | CA | GLU | 567 | −12.109 | 0.390 | −0.171 | 0.00 | 0.00 | 0.158 | 9.40 | 4.00 |
| ATOM | 3877 | HA | GLU | 567 | −11.422 | −0.227 | 0.408 | 0.00 | 0.00 | 0.053 | 0.00 | 0.00 |
| ATOM | 3878 | C | GLU | 567 | −12.793 | −0.473 | −1.179 | 0.00 | 0.00 | 0.396 | 9.82 | 4.00 |
| ATOM | 3879 | O | GLU | 567 | −12.578 | −1.682 | −1.215 | 0.00 | 0.00 | −0.396 | 8.17 | −17.40 |
| ATOM | 3880 | CB | GLU | 567 | −13.207 | 0.927 | 0.765 | 0.00 | 0.00 | −0.106 | 12.77 | 4.00 |
| ATOM | 3881 | HB1 | GLU | 567 | −13.909 | 1.510 | 0.169 | 0.00 | 0.00 | 0.053 | 0.00 | 0.00 |
| ATOM | 3882 | HB2 | GLU | 567 | −12.734 | 1.552 | 1.523 | 0.00 | 0.00 | 0.053 | 0.00 | 0.00 |
| ATOM | 3883 | CG | GLU | 567 | −14.005 | −0.157 | 1.491 | 0.00 | 0.00 | −0.336 | 12.77 | 4.00 |
| ATOM | 3884 | HG1 | GLU | 567 | −13.341 | −0.628 | 2.216 | 0.00 | 0.00 | 0.053 | 0.00 | 0.00 |
| ATOM | 3885 | HG2 | GLU | 567 | −14.345 | −0.875 | 0.744 | 0.00 | 0.00 | 0.053 | 0.00 | 0.00 |
| ATOM | 3886 | CD | GLU | 567 | −15.184 | 0.519 | 2.182 | 0.00 | 0.00 | 0.297 | 9.82 | 4.00 |
| ATOM | 3887 | OE1 | GLU | 567 | −15.875 | 1.320 | 1.499 | 0.00 | 0.00 | −0.534 | 8.17 | −18.95 |
| ATOM | 3888 | OE2 | GLU | 567 | −15.410 | 0.249 | 3.392 | 0.00 | 0.00 | −0.534 | 8.17 | −18.95 |
| ATOM | 3889 | N | VAL | 568 | −13.639 | 0.121 | −2.032 | 0.00 | 0.00 | −0.650 | 9.00 | −17.40 |
| ATOM | 3890 | HN | VAL | 568 | −13.777 | 1.141 | −2.012 | 0.00 | 0.00 | 0.439 | 0.00 | 0.00 |
| ATOM | 3891 | CA | VAL | 568 | −14.345 | −0.695 | −2.970 | 0.00 | 0.00 | 0.158 | 9.40 | 4.00 |
| ATOM | 3892 | HA | VAL | 568 | −14.712 | −1.600 | −2.486 | 0.00 | 0.00 | 0.053 | 0.00 | 0.00 |
| ATOM | 3893 | C | VAL | 568 | −13.445 | −1.091 | −4.098 | 0.00 | 0.00 | 0.396 | 9.82 | 4.00 |
| ATOM | 3894 | O | VAL | 568 | −13.352 | −2.268 | −4.442 | 0.00 | 0.00 | −0.396 | 8.17 | −17.40 |
| ATOM | 3895 | CB | VAL | 568 | −15.533 | 0.002 | −3.565 | 0.00 | 0.00 | −0.053 | 9.40 | 4.00 |
| ATOM | 3896 | HB | VAL | 568 | −15.187 | 0.903 | −4.072 | 0.00 | 0.00 | 0.053 | 0.00 | 0.00 |
| ATOM | 3897 | CG1 | VAL | 568 | −16.208 | −0.953 | −4.566 | 0.00 | 0.00 | −0.159 | 16.15 | 4.00 |
| ATOM | 3898 | HG11 | VAL | 568 | −17.075 | −0.463 | −5.009 | 0.00 | 0.00 | 0.053 | 0.00 | 0.00 |
| ATOM | 3899 | HG12 | VAL | 568 | −15.500 | −1.217 | −5.351 | 0.00 | 0.00 | 0.053 | 0.00 | 0.00 |
| ATOM | 3900 | HG13 | VAL | 568 | −16.528 | −1.857 | −4.047 | 0.00 | 0.00 | 0.053 | 0.00 | 0.00 |
| ATOM | 3901 | CG2 | VAL | 568 | −16.457 | 0.455 | −2.422 | 0.00 | 0.00 | −0.159 | 16.15 | 4.00 |
| ATOM | 3902 | HG21 | VAL | 568 | −17.327 | 0.964 | −2.837 | 0.00 | 0.00 | 0.053 | 0.00 | 0.00 |
| ATOM | 3903 | HG22 | VAL | 568 | −16.784 | −0.415 | −1.852 | 0.00 | 0.00 | 0.053 | 0.00 | 0.00 |
| ATOM | 3904 | HG23 | VAL | 568 | −15.916 | 1.137 | −1.766 | 0.00 | 0.00 | 0.053 | 0.00 | 0.00 |
| ATOM | 3905 | N | PHE | 569 | −12.735 | −0.114 | −4.694 | 0.00 | 0.00 | −0.650 | 9.00 | −17.40 |
| ATOM | 3906 | HN | PHE | 569 | −12.725 | 0.834 | −4.292 | 0.00 | 0.00 | 0.439 | 0.00 | 0.00 |
| ATOM | 3907 | CA | PHE | 569 | −11.996 | −0.393 | −5.884 | 0.00 | 0.00 | 0.158 | 9.40 | 4.00 |
| ATOM | 3908 | HA | PHE | 569 | −12.674 | −0.839 | −6.611 | 0.00 | 0.00 | 0.053 | 0.00 | 0.00 |
| ATOM | 3909 | C | PHE | 569 | −10.861 | −1.330 | −5.692 | 0.00 | 0.00 | 0.396 | 9.82 | 4.00 |
| ATOM | 3910 | O | PHE | 569 | −10.731 | −2.262 | −6.480 | 0.00 | 0.00 | −0.396 | 8.17 | −17.40 |
| ATOM | 3911 | CB | PHE | 569 | −11.533 | 0.861 | −6.631 | 0.00 | 0.00 | −0.106 | 12.77 | 4.00 |
| ATOM | 3912 | HB1 | PHE | 569 | −10.749 | 0.631 | −7.352 | 0.00 | 0.00 | 0.053 | 0.00 | 0.00 |
| ATOM | 3913 | HB2 | PHE | 569 | −11.135 | 1.610 | −5.947 | 0.00 | 0.00 | 0.053 | 0.00 | 0.00 |
| ATOM | 3914 | CG | PHE | 569 | −12.795 | 1.296 | −7.281 | 0.00 | 0.00 | 0.000 | 7.26 | 0.60 |
| ATOM | 3915 | CD1 | PHE | 569 | −13.156 | 0.777 | −8.502 | 0.00 | 0.00 | −0.127 | 10.80 | 0.60 |
| ATOM | 3916 | HD1 | PHE | 569 | −12.506 | 0.054 | −8.995 | 0.00 | 0.00 | 0.127 | 0.00 | 0.00 |
| ATOM | 3917 | CD2 | PHE | 569 | −13.632 | 2.187 | −6.659 | 0.00 | 0.00 | −0.127 | 10.80 | 0.60 |
| ATOM | 3918 | HD2 | PHE | 569 | −13.364 | 2.589 | −5.682 | 0.00 | 0.00 | 0.127 | 0.00 | 0.00 |
| ATOM | 3919 | CE1 | PHE | 569 | −14.328 | 1.162 | −9.106 | 0.00 | 0.00 | −0.127 | 10.80 | 0.60 |
| ATOM | 3920 | HE1 | PHE | 569 | −14.602 | 0.750 | −10.077 | 0.00 | 0.00 | 0.127 | 0.00 | 0.00 |
| ATOM | 3921 | CE2 | PHE | 569 | −14.806 | 2.577 | −7.259 | 0.00 | 0.00 | −0.127 | 10.80 | 0.60 |
| ATOM | 3922 | HE2 | PHE | 569 | −15.461 | 3.292 | −6.761 | 0.00 | 0.00 | 0.127 | 0.00 | 0.00 |
| ATOM | 3923 | CZ | PHE | 569 | −15.153 | 2.066 | −8.486 | 0.00 | 0.00 | −0.127 | 10.80 | 0.60 |
| ATOM | 3924 | HZ | PHE | 569 | −16.081 | 2.378 | −8.966 | 0.00 | 0.00 | 0.127 | 0.00 | 0.00 |
| ATOM | 3925 | N | ILE | 570 | −10.054 | −1.202 | −4.615 | 0.00 | 0.00 | −0.650 | 9.00 | −17.40 |
| ATOM | 3926 | HN | ILE | 570 | −10.257 | −0.508 | −3.882 | 0.00 | 0.00 | 0.439 | 0.00 | 0.00 |
| ATOM | 3927 | CA | ILE | 570 | −8.900 | −2.064 | −4.520 | 0.00 | 0.00 | 0.158 | 9.40 | 4.00 |
| ATOM | 3928 | HA | ILE | 570 | −8.327 | −1.912 | −5.434 | 0.00 | 0.00 | 0.053 | 0.00 | 0.00 |
| ATOM | 3929 | C | ILE | 570 | −9.422 | −3.452 | −4.403 | 0.00 | 0.00 | 0.396 | 9.82 | 4.00 |
| ATOM | 3930 | O | ILE | 570 | −8.710 | −4.429 | −4.622 | 0.00 | 0.00 | −0.396 | 8.17 | −17.40 |
| ATOM | 3931 | CB | ILE | 570 | −8.024 | −1.914 | −3.301 | 0.00 | 0.00 | −0.053 | 9.40 | 4.00 |
| ATOM | 3932 | HB | ILE | 570 | −7.077 | −2.412 | −3.510 | 0.00 | 0.00 | 0.053 | 0.00 | 0.00 |
| ATOM | 3933 | CG1 | ILE | 570 | −8.549 | −2.696 | −2.074 | 0.00 | 0.00 | −0.106 | 12.77 | 4.00 |
| ATOM | 3934 | HG11 | ILE | 570 | −7.876 | −2.484 | −1.243 | 0.00 | 0.00 | 0.053 | 0.00 | 0.00 |
| ATOM | 3935 | HG12 | ILE | 570 | −8.541 | −3.754 | −2.336 | 0.00 | 0.00 | 0.053 | 0.00 | 0.00 |
| ATOM | 3936 | CG2 | ILE | 570 | −7.818 | −0.411 | −3.056 | 0.00 | 0.00 | −0.159 | 16.15 | 4.00 |
| ATOM | 3937 | HG21 | ILE | 570 | −7.187 | −0.268 | −2.179 | 0.00 | 0.00 | 0.053 | 0.00 | 0.00 |
| ATOM | 3938 | HG22 | ILE | 570 | −7.337 | 0.037 | −3.926 | 0.00 | 0.00 | 0.053 | 0.00 | 0.00 |
| ATOM | 3939 | HG23 | ILE | 570 | −8.784 | 0.066 | −2.890 | 0.00 | 0.00 | 0.053 | 0.00 | 0.00 |
| ATOM | 3940 | CD1 | ILE | 570 | −9.954 | −2.393 | −1.572 | 0.00 | 0.00 | −0.159 | 16.15 | 4.00 |
| ATOM | 3941 | HD11 | ILE | 570 | −10.175 | −3.021 | −0.709 | 0.00 | 0.00 | 0.053 | 0.00 | 0.00 |
| ATOM | 3942 | HD12 | ILE | 570 | −10.019 | −1.344 | −1.284 | 0.00 | 0.00 | 0.053 | 0.00 | 0.00 |
| ATOM | 3943 | HD13 | ILE | 570 | −10.675 | −2.597 | −2.363 | 0.00 | 0.00 | 0.053 | 0.00 | 0.00 |
| ATOM | 3944 | N | GLY | 571 | −10.703 | −3.559 | −4.031 | 0.00 | 0.00 | −0.650 | 9.00 | −17.40 |
| ATOM | 3945 | HN | GLY | 571 | −11.269 | −2.709 | −3.897 | 0.00 | 0.00 | 0.439 | 0.00 | 0.00 |
| ATOM | 3946 | CA | GLY | 571 | −11.287 | −4.831 | −3.820 | 0.00 | 0.00 | 0.105 | 9.40 | 4.00 |
| ATOM | 3947 | HA1 | GLY | 571 | −10.745 | −5.613 | −4.352 | 0.00 | 0.00 | 0.053 | 0.00 | 0.00 |
| ATOM | 3948 | HA2 | GLY | 571 | −12.320 | −4.859 | −4.166 | 0.00 | 0.00 | 0.053 | 0.00 | 0.00 |
| ATOM | 3949 | C | GLY | 571 | −11.178 | −4.965 | −2.354 | 0.00 | 0.00 | 0.396 | 9.82 | 4.00 |
| ATOM | 3950 | O | GLY | 571 | −10.077 | −5.124 | −1.829 | 0.00 | 0.00 | −0.396 | 8.17 | −17.40 |
| ATOM | 3951 | N | ARG | 572 | −12.317 | −4.873 | −1.646 | 0.00 | 0.00 | −0.650 | 9.00 | −17.40 |
| ATOM | 3952 | HN | ARG | 572 | −13.233 | −4.750 | −2.101 | 0.00 | 0.00 | 0.439 | 0.00 | 0.00 |
| ATOM | 3953 | CA | ARG | 572 | −12.183 | −4.956 | −0.232 | 0.00 | 0.00 | 0.158 | 9.40 | 4.00 |
| ATOM | 3954 | HA | ARG | 572 | −11.507 | −4.162 | 0.085 | 0.00 | 0.00 | 0.053 | 0.00 | 0.00 |
| ATOM | 3955 | C | ARG | 572 | −11.623 | −6.302 | 0.094 | 0.00 | 0.00 | 0.351 | 9.82 | 4.00 |
| ATOM | 3956 | HC | ARG | 572 | −11.911 | −7.177 | −0.488 | 0.00 | 0.00 | 0.046 | 0.00 | 0.00 |
| ATOM | 3957 | O | ARG | 572 | −10.832 | −6.407 | 1.030 | 0.00 | 0.00 | −0.396 | 8.17 | −17.40 |
| ATOM | 3958 | CB | ARG | 572 | −13.448 | −4.583 | 0.589 | 0.00 | 0.00 | −0.106 | 12.77 | 4.00 |
| ATOM | 3959 | HB1 | ARG | 572 | −13.660 | −3.532 | 0.394 | 0.00 | 0.00 | 0.053 | 0.00 | 0.00 |
| ATOM | 3960 | HB2 | ARG | 572 | −13.214 | −4.758 | 1.639 | 0.00 | 0.00 | 0.053 | 0.00 | 0.00 |
| ATOM | 3961 | CG | ARG | 572 | −14.750 | −5.336 | 0.314 | 0.00 | 0.00 | −0.106 | 12.77 | 4.00 |
| ATOM | 3962 | HG1 | ARG | 572 | −14.704 | −6.393 | 0.577 | 0.00 | 0.00 | 0.053 | 0.00 | 0.00 |
| ATOM | 3963 | HG2 | ARG | 572 | −15.051 | −5.315 | −0.733 | 0.00 | 0.00 | 0.053 | 0.00 | 0.00 |
| ATOM | 3964 | CD | ARG | 572 | −15.948 | −4.783 | 1.086 | 0.00 | 0.00 | 0.374 | 12.77 | 4.00 |
| ATOM | 3965 | HD1 | ARG | 572 | −15.631 | −4.656 | 2.121 | 0.00 | 0.00 | 0.053 | 0.00 | 0.00 |
| ATOM | 3966 | HD2 | ARG | 572 | −16.752 | −5.514 | 0.996 | 0.00 | 0.00 | 0.053 | 0.00 | 0.00 |
| ATOM | 3967 | NE | ARG | 572 | −16.312 | −3.482 | 0.457 | 0.00 | 0.00 | −0.819 | 9.00 | −24.67 |
| ATOM | 3968 | HE | ARG | 572 | −15.775 | −3.134 | −0.350 | 0.00 | 0.00 | 0.407 | 0.00 | 0.00 |
| ATOM | 3969 | CZ | ARG | 572 | −17.355 | −2.751 | 0.948 | 0.00 | 0.00 | 0.796 | 6.95 | 4.00 |
| ATOM | 3970 | NH1 | ARG | 572 | −18.058 | −3.203 | 2.027 | 0.00 | 0.00 | −0.746 | 9.00 | −24.67 |
| ATOM | 3971 | HH11 | ARG | 572 | −18.845 | −2.651 | 2.397 | 0.00 | 0.00 | 0.407 | 0.00 | 0.00 |
| ATOM | 3972 | HH12 | ARG | 572 | −17.802 | −4.096 | 2.471 | 0.00 | 0.00 | 0.407 | 0.00 | 0.00 |
| ATOM | 3973 | NH2 | ARG | 572 | −17.693 | −1.567 | 0.360 | 0.00 | 0.00 | −0.746 | 9.00 | −24.67 |
| ATOM | 3974 | HH21 | ARG | 572 | −18.480 | −1.015 | 0.730 | 0.00 | 0.00 | 0.407 | 0.00 | 0.00 |
| ATOM | 3975 | HH22 | ARG | 572 | −17.161 | −1.226 | −0.453 | 0.00 | 0.00 | 0.407 | 0.00 | 0.00 |
| ATOM | 3976 | N | VAL | 578 | −8.752 | −5.496 | 4.776 | 0.00 | 0.00 | −0.580 | 9.00 | −17.40 |
| ATOM | 3977 | HN2 | VAL | 578 | −8.032 | −5.037 | 5.353 | 0.00 | 0.00 | 0.249 | 0.00 | 0.00 |
| ATOM | 3978 | HN1 | VAL | 578 | −9.146 | −6.262 | 5.340 | 0.00 | 0.00 | 0.249 | 0.00 | 0.00 |
| ATOM | 3979 | CA | VAL | 578 | −9.680 | −4.483 | 4.341 | 0.00 | 0.00 | 0.030 | 9.40 | 4.00 |
| ATOM | 3980 | HA | VAL | 578 | −9.565 | −4.236 | 3.286 | 0.00 | 0.00 | 0.053 | 0.00 | 0.00 |
| ATOM | 3981 | C | VAL | 578 | −9.519 | −3.195 | 5.093 | 0.00 | 0.00 | 0.396 | 9.82 | 4.00 |
| ATOM | 3982 | O | VAL | 578 | −9.875 | −2.130 | 4.588 | 0.00 | 0.00 | −0.396 | 8.17 | −17.40 |
| ATOM | 3983 | CB | VAL | 578 | −11.111 | −4.910 | 4.503 | 0.00 | 0.00 | −0.053 | 9.40 | 4.00 |
| ATOM | 3984 | HB | VAL | 578 | −11.293 | −5.133 | 5.554 | 0.00 | 0.00 | 0.053 | 0.00 | 0.00 |
| ATOM | 3985 | CG1 | VAL | 578 | −12.025 | −3.762 | 4.037 | 0.00 | 0.00 | −0.159 | 16.15 | 4.00 |
| ATOM | 3986 | HG11 | VAL | 578 | −13.068 | −4.059 | 4.149 | 0.00 | 0.00 | 0.053 | 0.00 | 0.00 |
| ATOM | 3987 | HG12 | VAL | 578 | −11.833 | −2.876 | 4.642 | 0.00 | 0.00 | 0.053 | 0.00 | 0.00 |
| ATOM | 3988 | HG13 | VAL | 578 | −11.822 | −3.538 | 2.990 | 0.00 | 0.00 | 0.053 | 0.00 | 0.00 |
| ATOM | 3989 | CG2 | VAL | 578 | −11.316 | −6.226 | 3.730 | 0.00 | 0.00 | −0.159 | 16.15 | 4.00 |
| ATOM | 3990 | HG21 | VAL | 578 | −12.351 | −6.553 | 3.836 | 0.00 | 0.00 | 0.053 | 0.00 | 0.00 |
| ATOM | 3991 | HG22 | VAL | 578 | −11.092 | −6.067 | 2.675 | 0.00 | 0.00 | 0.053 | 0.00 | 0.00 |
| ATOM | 3992 | HG23 | VAL | 578 | −10.651 | −6.991 | 4.131 | 0.00 | 0.00 | 0.053 | 0.00 | 0.00 |
| ATOM | 3993 | N | ASP | 579 | −9.013 | −3.248 | 6.336 | 0.00 | 0.00 | −0.650 | 9.00 | −17.40 |
| ATOM | 3994 | HN | ASP | 579 | −8.654 | −4.137 | 6.714 | 0.00 | 0.00 | 0.439 | 0.00 | 0.00 |
| ATOM | 3995 | CA | ASP | 579 | −8.977 | −2.054 | 7.134 | 0.00 | 0.00 | 0.158 | 9.40 | 4.00 |
| ATOM | 3996 | HA | ASP | 579 | −9.990 | −1.690 | 7.308 | 0.00 | 0.00 | 0.053 | 0.00 | 0.00 |
| ATOM | 3997 | C | ASP | 579 | −8.193 | −0.989 | 6.430 | 0.00 | 0.00 | 0.396 | 9.82 | 4.00 |
| ATOM | 3998 | O | ASP | 579 | −8.634 | 0.156 | 6.351 | 0.00 | 0.00 | −0.396 | 8.17 | −17.40 |
| ATOM | 3999 | CB | ASP | 579 | −8.321 | −2.272 | 8.508 | 0.00 | 0.00 | −0.336 | 12.77 | 4.00 |
| ATOM | 4000 | HB1 | ASP | 579 | −7.252 | −2.424 | 8.357 | 0.00 | 0.00 | 0.053 | 0.00 | 0.00 |
| ATOM | 4001 | HB2 | ASP | 579 | −8.773 | −3.152 | 8.967 | 0.00 | 0.00 | 0.053 | 0.00 | 0.00 |
| ATOM | 4002 | CG | ASP | 579 | −8.572 | −1.033 | 9.360 | 0.00 | 0.00 | 0.297 | 9.82 | 4.00 |
| ATOM | 4003 | OD1 | ASP | 579 | −9.160 | −0.055 | 8.826 | 0.00 | 0.00 | −0.534 | 8.17 | −18.95 |
| ATOM | 4004 | OD2 | ASP | 579 | −8.178 | −1.048 | 10.557 | 0.00 | 0.00 | −0.534 | 8.17 | −18.95 |
| ATOM | 4005 | N | LEU | 580 | −7.025 | −1.341 | 5.867 | 0.00 | 0.00 | −0.650 | 9.00 | −17.40 |
| ATOM | 4006 | HN | LEU | 580 | −6.724 | −2.326 | 5.882 | 0.00 | 0.00 | 0.439 | 0.00 | 0.00 |
| ATOM | 4007 | CA | LEU | 580 | −6.196 | −0.350 | 5.246 | 0.00 | 0.00 | 0.158 | 9.40 | 4.00 |
| ATOM | 4008 | HA | LEU | 580 | −5.920 | 0.408 | 5.980 | 0.00 | 0.00 | 0.053 | 0.00 | 0.00 |
| ATOM | 4009 | C | LEU | 580 | −6.944 | 0.297 | 4.121 | 0.00 | 0.00 | 0.396 | 9.82 | 4.00 |
| ATOM | 4010 | O | LEU | 580 | −6.917 | 1.519 | 3.977 | 0.00 | 0.00 | −0.396 | 8.17 | −17.40 |
| ATOM | 4011 | CB | LEU | 580 | −4.891 | −0.941 | 4.683 | 0.00 | 0.00 | −0.106 | 12.77 | 4.00 |
| ATOM | 4012 | HB1 | LEU | 580 | −4.315 | −0.230 | 4.091 | 0.00 | 0.00 | 0.053 | 0.00 | 0.00 |
| ATOM | 4013 | HB2 | LEU | 580 | −5.052 | −1.797 | 4.028 | 0.00 | 0.00 | 0.053 | 0.00 | 0.00 |
| ATOM | 4014 | CG | LEU | 580 | −3.925 | −1.439 | 5.776 | 0.00 | 0.00 | −0.053 | 9.40 | 4.00 |
| ATOM | 4015 | HG | LEU | 580 | −3.081 | −1.976 | 5.343 | 0.00 | 0.00 | 0.053 | 0.00 | 0.00 |
| ATOM | 4016 | CD1 | LEU | 580 | −3.360 | −0.268 | 6.597 | 0.00 | 0.00 | −0.159 | 16.15 | 4.00 |
| ATOM | 4017 | HD11 | LEU | 580 | −2.682 | −0.652 | 7.360 | 0.00 | 0.00 | 0.053 | 0.00 | 0.00 |
| ATOM | 4018 | HD12 | LEU | 580 | −2.818 | 0.410 | 5.938 | 0.00 | 0.00 | 0.053 | 0.00 | 0.00 |
| ATOM | 4019 | HD13 | LEU | 580 | −4.179 | 0.269 | 7.076 | 0.00 | 0.00 | 0.053 | 0.00 | 0.00 |
| ATOM | 4020 | CD2 | LEU | 580 | −4.580 | −2.512 | 6.662 | 0.00 | 0.00 | −0.159 | 16.15 | 4.00 |
| ATOM | 4021 | HD21 | LEU | 580 | −3.870 | −2.840 | 7.421 | 0.00 | 0.00 | 0.053 | 0.00 | 0.00 |
| ATOM | 4022 | HD22 | LEU | 580 | −5.463 | −2.095 | 7.147 | 0.00 | 0.00 | 0.053 | 0.00 | 0.00 |
| ATOM | 4023 | HD23 | LEU | 580 | −4.872 | −3.363 | 6.047 | 0.00 | 0.00 | 0.053 | 0.00 | 0.00 |
| ATOM | 4024 | N | ASN | 581 | −7.656 | −0.492 | 3.295 | 0.00 | 0.00 | −0.650 | 9.00 | −17.40 |
| ATOM | 4025 | HN | ASN | 581 | −7.701 | −1.512 | 3.435 | 0.00 | 0.00 | 0.439 | 0.00 | 0.00 |
| ATOM | 4026 | CA | ASN | 581 | −8.348 | 0.143 | 2.215 | 0.00 | 0.00 | 0.158 | 9.40 | 4.00 |
| ATOM | 4027 | HA | ASN | 581 | −7.614 | 0.690 | 1.623 | 0.00 | 0.00 | 0.053 | 0.00 | 0.00 |
| ATOM | 4028 | C | ASN | 581 | −9.358 | 1.067 | 2.801 | 0.00 | 0.00 | 0.396 | 9.82 | 4.00 |
| ATOM | 4029 | O | ASN | 581 | −9.577 | 2.164 | 2.290 | 0.00 | 0.00 | −0.396 | 8.17 | −17.40 |
| ATOM | 4030 | CB | ASN | 581 | −9.043 | −0.820 | 1.223 | 0.00 | 0.00 | −0.106 | 12.77 | 4.00 |
| ATOM | 4031 | HB1 | ASN | 581 | −8.340 | −1.519 | 0.769 | 0.00 | 0.00 | 0.053 | 0.00 | 0.00 |
| ATOM | 4032 | HB2 | ASN | 581 | −9.527 | −0.287 | 0.405 | 0.00 | 0.00 | 0.053 | 0.00 | 0.00 |
| ATOM | 4033 | CG | ASN | 581 | −10.120 | −1.658 | 1.898 | 0.00 | 0.00 | 0.396 | 9.82 | 4.00 |
| ATOM | 4034 | OD1 | ASN | 581 | −9.918 | −2.843 | 2.153 | 0.00 | 0.00 | −0.396 | 8.17 | −17.40 |
| ATOM | 4035 | ND2 | ASN | 581 | −11.299 | −1.042 | 2.175 | 0.00 | 0.00 | −0.879 | 13.25 | −17.40 |
| ATOM | 4036 | HD21 | ASN | 581 | −11.426 | −0.046 | 1.945 | 0.00 | 0.00 | 0.439 | 0.00 | 0.00 |
| ATOM | 4037 | HD22 | ASN | 581 | −12.065 | −1.571 | 2.615 | 0.00 | 0.00 | 0.439 | 0.00 | 0.00 |
| ATOM | 4038 | N | ASN | 582 | −9.999 | 0.644 | 3.904 | 0.00 | 0.00 | −0.650 | 9.00 | −17.40 |
| ATOM | 4039 | HN | ASN | 582 | −9.776 | −0.274 | 4.315 | 0.00 | 0.00 | 0.439 | 0.00 | 0.00 |
| ATOM | 4040 | CA | ASN | 582 | −10.992 | 1.480 | 4.500 | 0.00 | 0.00 | 0.158 | 9.40 | 4.00 |
| ATOM | 4041 | HA | ASN | 582 | −11.757 | 1.737 | 3.768 | 0.00 | 0.00 | 0.053 | 0.00 | 0.00 |
| ATOM | 4042 | C | ASN | 582 | −10.368 | 2.745 | 5.008 | 0.00 | 0.00 | 0.396 | 9.82 | 4.00 |
| ATOM | 4043 | O | ASN | 582 | −10.850 | 3.834 | 4.701 | 0.00 | 0.00 | −0.396 | 8.17 | −17.40 |
| ATOM | 4044 | CB | ASN | 582 | −11.689 | 0.825 | 5.707 | 0.00 | 0.00 | −0.106 | 12.77 | 4.00 |
| ATOM | 4045 | HB1 | ASN | 582 | −12.291 | 1.578 | 6.215 | 0.00 | 0.00 | 0.053 | 0.00 | 0.00 |
| ATOM | 4046 | HB2 | ASN | 582 | −10.928 | 0.434 | 6.382 | 0.00 | 0.00 | 0.053 | 0.00 | 0.00 |
| ATOM | 4047 | CG | ASN | 582 | −12.576 | −0.306 | 5.206 | 0.00 | 0.00 | 0.396 | 9.82 | 4.00 |
| ATOM | 4048 | OD1 | ASN | 582 | −13.022 | −0.296 | 4.060 | 0.00 | 0.00 | −0.396 | 8.17 | −17.40 |
| ATOM | 4049 | ND2 | ASN | 582 | −12.851 | −1.303 | 6.088 | 0.00 | 0.00 | −0.879 | 13.25 | −17.40 |
| ATOM | 4050 | HD21 | ASN | 582 | −12.457 | −1.273 | 7.039 | 0.00 | 0.00 | 0.439 | 0.00 | 0.00 |
| ATOM | 4051 | HD22 | ASN | 582 | −13.454 | −2.089 | 5.805 | 0.00 | 0.00 | 0.439 | 0.00 | 0.00 |
| ATOM | 4052 | N | GLN | 583 | −9.320 | 2.635 | 5.854 | 0.00 | 0.00 | −0.650 | 9.00 | −17.40 |
| ATOM | 4053 | HN | GLN | 583 | −8.955 | 1.701 | 6.090 | 0.00 | 0.00 | 0.439 | 0.00 | 0.00 |
| ATOM | 4054 | CA | GLN | 583 | −8.706 | 3.801 | 6.430 | 0.00 | 0.00 | 0.158 | 9.40 | 4.00 |
| ATOM | 4055 | HA | GLN | 583 | −9.498 | 4.494 | 6.713 | 0.00 | 0.00 | 0.053 | 0.00 | 0.00 |
| ATOM | 4056 | C | GLN | 583 | −7.760 | 4.550 | 5.525 | 0.00 | 0.00 | 0.396 | 9.82 | 4.00 |
| ATOM | 4057 | O | GLN | 583 | −7.947 | 5.741 | 5.282 | 0.00 | 0.00 | −0.396 | 8.17 | −17.40 |
| ATOM | 4058 | CB | GLN | 583 | −7.920 | 3.472 | 7.711 | 0.00 | 0.00 | −0.106 | 12.77 | 4.00 |
| ATOM | 4059 | HB1 | GLN | 583 | −7.357 | 4.317 | 8.106 | 0.00 | 0.00 | 0.053 | 0.00 | 0.00 |
| ATOM | 4060 | HB2 | GLN | 583 | −7.185 | 2.678 | 7.580 | 0.00 | 0.00 | 0.053 | 0.00 | 0.00 |
| ATOM | 4061 | CG | GLN | 583 | −8.805 | 3.007 | 8.870 | 0.00 | 0.00 | −0.106 | 12.77 | 4.00 |
| ATOM | 4062 | HG1 | GLN | 583 | −8.150 | 2.647 | 9.663 | 0.00 | 0.00 | 0.053 | 0.00 | 0.00 |
| ATOM | 4063 | HG2 | GLN | 583 | −9.447 | 2.209 | 8.497 | 0.00 | 0.00 | 0.053 | 0.00 | 0.00 |
| ATOM | 4064 | CD | GLN | 583 | −9.626 | 4.203 | 9.334 | 0.00 | 0.00 | 0.396 | 9.82 | 4.00 |
| ATOM | 4065 | OE1 | GLN | 583 | −9.964 | 5.080 | 8.541 | 0.00 | 0.00 | −0.396 | 8.17 | −17.40 |
| ATOM | 4066 | NE2 | GLN | 583 | −9.952 | 4.246 | 10.653 | 0.00 | 0.00 | −0.879 | 13.25 | −17.40 |
| ATOM | 4067 | HE21 | GLN | 583 | −9.649 | 3.490 | 11.283 | 0.00 | 0.00 | 0.439 | 0.00 | 0.00 |
| ATOM | 4068 | HE22 | GLN | 583 | −10.502 | 5.035 | 11.022 | 0.00 | 0.00 | 0.439 | 0.00 | 0.00 |
| ATOM | 4069 | N | GLU | 584 | −6.660 | 3.888 | 5.095 | 0.00 | 0.00 | −0.650 | 9.00 | −17.40 |
| ATOM | 4070 | HN | GLU | 584 | −6.560 | 2.893 | 5.344 | 0.00 | 0.00 | 0.439 | 0.00 | 0.00 |
| ATOM | 4071 | CA | GLU | 584 | −5.617 | 4.505 | 4.304 | 0.00 | 0.00 | 0.158 | 9.40 | 4.00 |
| ATOM | 4072 | HA | GLU | 584 | −5.546 | 5.549 | 4.608 | 0.00 | 0.00 | 0.053 | 0.00 | 0.00 |
| ATOM | 4073 | C | GLU | 584 | −5.804 | 4.499 | 2.810 | 0.00 | 0.00 | 0.396 | 9.82 | 4.00 |
| ATOM | 4074 | O | GLU | 584 | −5.695 | 5.535 | 2.154 | 0.00 | 0.00 | −0.396 | 8.17 | −17.40 |
| ATOM | 4075 | CB | GLU | 584 | −4.237 | 3.876 | 4.559 | 0.00 | 0.00 | −0.106 | 12.77 | 4.00 |
| ATOM | 4076 | HB1 | GLU | 584 | −3.927 | 3.912 | 5.603 | 0.00 | 0.00 | 0.053 | 0.00 | 0.00 |
| ATOM | 4077 | HB2 | GLU | 584 | −3.429 | 4.357 | 4.008 | 0.00 | 0.00 | 0.053 | 0.00 | 0.00 |
| ATOM | 4078 | CG | GLU | 584 | −4.156 | 2.397 | 4.172 | 0.00 | 0.00 | −0.336 | 12.77 | 4.00 |
| ATOM | 4079 | HG1 | GLU | 584 | −4.434 | 2.307 | 3.122 | 0.00 | 0.00 | 0.053 | 0.00 | 0.00 |
| ATOM | 4080 | HG2 | GLU | 584 | −4.849 | 1.843 | 4.806 | 0.00 | 0.00 | 0.053 | 0.00 | 0.00 |
| ATOM | 4081 | CD | GLU | 584 | −2.726 | 1.929 | 4.395 | 0.00 | 0.00 | 0.297 | 9.82 | 4.00 |
| ATOM | 4082 | OE1 | GLU | 584 | −1.901 | 2.755 | 4.872 | 0.00 | 0.00 | −0.534 | 8.17 | −18.95 |
| ATOM | 4083 | OE2 | GLU | 584 | −2.437 | 0.741 | 4.091 | 0.00 | 0.00 | −0.534 | 8.17 | −18.95 |
| ATOM | 4084 | N | GLU | 585 | −6.145 | 3.323 | 2.244 | 0.00 | 0.00 | −0.650 | 9.00 | −17.40 |
| ATOM | 4085 | HN | GLU | 585 | −6.462 | 2.559 | 2.858 | 0.00 | 0.00 | 0.439 | 0.00 | 0.00 |
| ATOM | 4086 | CA | GLU | 585 | −6.089 | 3.079 | 0.825 | 0.00 | 0.00 | 0.158 | 9.40 | 4.00 |
| ATOM | 4087 | HA | GLU | 585 | −5.058 | 3.189 | 0.487 | 0.00 | 0.00 | 0.053 | 0.00 | 0.00 |
| ATOM | 4088 | C | GLU | 585 | −6.954 | 4.059 | 0.135 | 0.00 | 0.00 | 0.396 | 9.82 | 4.00 |
| ATOM | 4089 | O | GLU | 585 | −6.646 | 4.498 | −0.972 | 0.00 | 0.00 | −0.396 | 8.17 | −17.40 |
| ATOM | 4090 | CB | GLU | 585 | −6.486 | 1.638 | 0.467 | 0.00 | 0.00 | −0.106 | 12.77 | 4.00 |
| ATOM | 4091 | HB1 | GLU | 585 | −6.503 | 1.504 | −0.615 | 0.00 | 0.00 | 0.053 | 0.00 | 0.00 |
| ATOM | 4092 | HB2 | GLU | 585 | −7.477 | 1.406 | 0.858 | 0.00 | 0.00 | 0.053 | 0.00 | 0.00 |
| ATOM | 4093 | CG | GLU | 585 | −5.485 | 0.641 | 1.063 | 0.00 | 0.00 | −0.336 | 12.77 | 4.00 |
| ATOM | 4094 | HG1 | GLU | 585 | −5.465 | 0.729 | 2.149 | 0.00 | 0.00 | 0.053 | 0.00 | 0.00 |
| ATOM | 4095 | HG2 | GLU | 585 | −4.484 | 0.836 | 0.679 | 0.00 | 0.00 | 0.053 | 0.00 | 0.00 |
| ATOM | 4096 | CD | GLU | 585 | −5.878 | −0.783 | 0.698 | 0.00 | 0.00 | 0.297 | 9.82 | 4.00 |
| ATOM | 4097 | OE1 | GLU | 585 | −6.720 | −0.959 | −0.222 | 0.00 | 0.00 | −0.534 | 8.17 | −18.95 |
| ATOM | 4098 | OE2 | GLU | 585 | −5.323 | −1.719 | 1.334 | 0.00 | 0.00 | −0.534 | 8.17 | −18.95 |
| ATOM | 4099 | N | VAL | 586 | −8.051 | 4.382 | 0.832 | 0.00 | 0.00 | −0.650 | 9.00 | −17.40 |
| ATOM | 4100 | HN | VAL | 586 | −8.207 | 3.793 | 1.663 | 0.00 | 0.00 | 0.439 | 0.00 | 0.00 |
| ATOM | 4101 | CA | VAL | 586 | −9.046 | 5.390 | 0.640 | 0.00 | 0.00 | 0.158 | 9.40 | 4.00 |
| ATOM | 4102 | HA | VAL | 586 | −10.022 | 4.921 | 0.515 | 0.00 | 0.00 | 0.053 | 0.00 | 0.00 |
| ATOM | 4103 | C | VAL | 586 | −8.718 | 6.196 | −0.590 | 0.00 | 0.00 | 0.396 | 9.82 | 4.00 |
| ATOM | 4104 | O | VAL | 586 | −7.705 | 6.891 | −0.640 | 0.00 | 0.00 | −0.396 | 8.17 | −17.40 |
| ATOM | 4105 | CB | VAL | 586 | −8.950 | 6.142 | 1.941 | 0.00 | 0.00 | −0.053 | 9.40 | 4.00 |
| ATOM | 4106 | HB | VAL | 586 | −7.933 | 6.528 | 2.015 | 0.00 | 0.00 | 0.053 | 0.00 | 0.00 |
| ATOM | 4107 | CG1 | VAL | 586 | −9.875 | 7.339 | 2.171 | 0.00 | 0.00 | −0.159 | 16.15 | 4.00 |
| ATOM | 4108 | HG11 | VAL | 586 | −9.678 | 7.767 | 3.154 | 0.00 | 0.00 | 0.053 | 0.00 | 0.00 |
| ATOM | 4109 | HG12 | VAL | 586 | −9.693 | 8.092 | 1.404 | 0.00 | 0.00 | 0.053 | 0.00 | 0.00 |
| ATOM | 4110 | HG13 | VAL | 586 | −10.913 | 7.012 | 2.119 | 0.00 | 0.00 | 0.053 | 0.00 | 0.00 |
| ATOM | 4111 | CG2 | VAL | 586 | −9.136 | 5.018 | 2.948 | 0.00 | 0.00 | −0.159 | 16.15 | 4.00 |
| ATOM | 4112 | HG21 | VAL | 586 | −9.089 | 5.423 | 3.959 | 0.00 | 0.00 | 0.053 | 0.00 | 0.00 |
| ATOM | 4113 | HG22 | VAL | 586 | −10.106 | 4.546 | 2.791 | 0.00 | 0.00 | 0.053 | 0.00 | 0.00 |
| ATOM | 4114 | HG23 | VAL | 586 | −8.347 | 4.278 | 2.817 | 0.00 | 0.00 | 0.053 | 0.00 | 0.00 |
| ATOM | 4115 | N | GLU | 587 | −9.534 | 6.023 | −1.657 | 0.00 | 0.00 | −0.650 | 9.00 | −17.40 |
| ATOM | 4116 | HN | GLU | 587 | −10.308 | 5.349 | −1.565 | 0.00 | 0.00 | 0.439 | 0.00 | 0.00 |
| ATOM | 4117 | CA | GLU | 587 | −9.394 | 6.717 | −2.912 | 0.00 | 0.00 | 0.158 | 9.40 | 4.00 |
| ATOM | 4118 | HA | GLU | 587 | −8.661 | 7.505 | −2.740 | 0.00 | 0.00 | 0.053 | 0.00 | 0.00 |
| ATOM | 4119 | C | GLU | 587 | −10.760 | 7.256 | −3.243 | 0.00 | 0.00 | 0.396 | 9.82 | 4.00 |
| ATOM | 4120 | O | GLU | 587 | −11.732 | 6.850 | −2.627 | 0.00 | 0.00 | −0.396 | 8.17 | −17.40 |
| ATOM | 4121 | CB | GLU | 587 | −8.896 | 5.799 | −4.039 | 0.00 | 0.00 | −0.106 | 12.77 | 4.00 |
| ATOM | 4122 | HB1 | GLU | 587 | −9.591 | 4.998 | −4.291 | 0.00 | 0.00 | 0.053 | 0.00 | 0.00 |
| ATOM | 4123 | HB2 | GLU | 587 | −7.957 | 5.295 | −3.811 | 0.00 | 0.00 | 0.053 | 0.00 | 0.00 |
| ATOM | 4124 | CG | GLU | 587 | −8.642 | 6.526 | −5.355 | 0.00 | 0.00 | −0.336 | 12.77 | 4.00 |
| ATOM | 4125 | HG1 | GLU | 587 | −7.795 | 6.023 | −5.821 | 0.00 | 0.00 | 0.053 | 0.00 | 0.00 |
| ATOM | 4126 | HG2 | GLU | 587 | −8.422 | 7.561 | −5.092 | 0.00 | 0.00 | 0.053 | 0.00 | 0.00 |
| ATOM | 4127 | CD | GLU | 587 | −9.915 | 6.390 | −6.164 | 0.00 | 0.00 | 0.297 | 9.82 | 4.00 |
| ATOM | 4128 | OE1 | GLU | 587 | −10.722 | 5.487 | −5.824 | 0.00 | 0.00 | −0.534 | 8.17 | −18.95 |
| ATOM | 4129 | OE2 | GLU | 587 | −10.085 | 7.155 | −7.149 | 0.00 | 0.00 | −0.534 | 8.17 | −18.95 |
| ATOM | 4130 | N | ASP | 588 | −10.912 | 8.168 | −4.223 | 0.00 | 0.00 | −0.650 | 9.00 | −17.40 |
| ATOM | 4131 | HN | ASP | 588 | −10.123 | 8.377 | −4.852 | 0.00 | 0.00 | 0.439 | 0.00 | 0.00 |
| ATOM | 4132 | CA | ASP | 588 | −12.169 | 8.855 | −4.401 | 0.00 | 0.00 | 0.158 | 9.40 | 4.00 |
| ATOM | 4133 | HA | ASP | 588 | −12.528 | 9.287 | −3.467 | 0.00 | 0.00 | 0.053 | 0.00 | 0.00 |
| ATOM | 4134 | C | ASP | 588 | −13.229 | 7.941 | −4.897 | 0.00 | 0.00 | 0.396 | 9.82 | 4.00 |
| ATOM | 4135 | O | ASP | 588 | −14.390 | 8.068 | −4.510 | 0.00 | 0.00 | −0.396 | 8.17 | −17.40 |
| ATOM | 4136 | CB | ASP | 588 | −12.130 | 10.026 | −5.390 | 0.00 | 0.00 | −0.336 | 12.77 | 4.00 |
| ATOM | 4137 | HB1 | ASP | 588 | −12.115 | 9.615 | −6.399 | 0.00 | 0.00 | 0.053 | 0.00 | 0.00 |
| ATOM | 4138 | HB2 | ASP | 588 | −11.227 | 10.604 | −5.193 | 0.00 | 0.00 | 0.053 | 0.00 | 0.00 |
| ATOM | 4139 | CG | ASP | 588 | −13.377 | 10.873 | −5.167 | 0.00 | 0.00 | 0.297 | 9.82 | 4.00 |
| ATOM | 4140 | OD1 | ASP | 588 | −14.051 | 10.668 | −4.121 | 0.00 | 0.00 | −0.534 | 8.17 | −18.95 |
| ATOM | 4141 | OD2 | ASP | 588 | −13.668 | 11.740 | −6.033 | 0.00 | 0.00 | −0.534 | 8.17 | −18.95 |
| ATOM | 4142 | N | ALA | 589 | −12.854 | 6.998 | −5.776 | 0.00 | 0.00 | −0.650 | 9.00 | −17.40 |
| ATOM | 4143 | HN | ALA | 589 | −11.863 | 6.924 | −6.048 | 0.00 | 0.00 | 0.439 | 0.00 | 0.00 |
| ATOM | 4144 | CA | ALA | 589 | −13.807 | 6.094 | −6.341 | 0.00 | 0.00 | 0.158 | 9.40 | 4.00 |
| ATOM | 4145 | HA | ALA | 589 | −14.497 | 6.709 | −6.918 | 0.00 | 0.00 | 0.053 | 0.00 | 0.00 |
| ATOM | 4146 | C | ALA | 589 | −14.463 | 5.419 | −5.189 | 0.00 | 0.00 | 0.396 | 9.82 | 4.00 |
| ATOM | 4147 | O | ALA | 589 | −15.645 | 5.081 | −5.237 | 0.00 | 0.00 | −0.396 | 8.17 | −17.40 |
| ATOM | 4148 | CB | ALA | 589 | −13.153 | 5.031 | −7.237 | 0.00 | 0.00 | −0.159 | 16.15 | 4.00 |
| ATOM | 4149 | HB1 | ALA | 589 | −13.922 | 4.371 | −7.640 | 0.00 | 0.00 | 0.053 | 0.00 | 0.00 |
| ATOM | 4150 | HB2 | ALA | 589 | −12.628 | 5.520 | −8.058 | 0.00 | 0.00 | 0.053 | 0.00 | 0.00 |
| ATOM | 4151 | HB3 | ALA | 589 | −12.444 | 4.446 | −6.650 | 0.00 | 0.00 | 0.053 | 0.00 | 0.00 |
| ATOM | 4152 | N | VAL | 590 | −13.695 | 5.228 | −4.105 | 0.00 | 0.00 | −0.650 | 9.00 | −17.40 |
| ATOM | 4153 | HN | VAL | 590 | −12.702 | 5.500 | −4.129 | 0.00 | 0.00 | 0.439 | 0.00 | 0.00 |
| ATOM | 4154 | CA | VAL | 590 | −14.242 | 4.651 | −2.920 | 0.00 | 0.00 | 0.158 | 9.40 | 4.00 |
| ATOM | 4155 | HA | VAL | 590 | −15.305 | 4.504 | −3.114 | 0.00 | 0.00 | 0.053 | 0.00 | 0.00 |
| ATOM | 4156 | C | VAL | 590 | −13.993 | 5.632 | −1.823 | 0.00 | 0.00 | 0.396 | 9.82 | 4.00 |
| ATOM | 4157 | O | VAL | 590 | −14.647 | 6.672 | −1.757 | 0.00 | 0.00 | −0.396 | 8.17 | −17.40 |
| ATOM | 4158 | CB | VAL | 590 | −13.637 | 3.339 | −2.532 | 0.00 | 0.00 | −0.053 | 9.40 | 4.00 |
| ATOM | 4159 | HB | VAL | 590 | −12.569 | 3.478 | −2.363 | 0.00 | 0.00 | 0.053 | 0.00 | 0.00 |
| ATOM | 4160 | CG1 | VAL | 590 | −14.313 | 2.839 | −1.243 | 0.00 | 0.00 | −0.159 | 16.15 | 4.00 |
| ATOM | 4161 | HG11 | VAL | 590 | −13.879 | 1.883 | −0.952 | 0.00 | 0.00 | 0.053 | 0.00 | 0.00 |
| ATOM | 4162 | HG12 | VAL | 590 | −14.158 | 3.566 | −0.445 | 0.00 | 0.00 | 0.053 | 0.00 | 0.00 |
| ATOM | 4163 | HG13 | VAL | 590 | −15.382 | 2.714 | −1.418 | 0.00 | 0.00 | 0.053 | 0.00 | 0.00 |
| ATOM | 4164 | CG2 | VAL | 590 | −13.782 | 2.378 | −3.721 | 0.00 | 0.00 | −0.159 | 16.15 | 4.00 |
| ATOM | 4165 | HG21 | VAL | 590 | −13.348 | 1.412 | −3.463 | 0.00 | 0.00 | 0.053 | 0.00 | 0.00 |
| ATOM | 4166 | HG22 | VAL | 590 | −14.838 | 2.249 | −3.958 | 0.00 | 0.00 | 0.053 | 0.00 | 0.00 |
| ATOM | 4167 | HG23 | VAL | 590 | −13.263 | 2.790 | −4.587 | 0.00 | 0.00 | 0.053 | 0.00 | 0.00 |
| ATOM | 4168 | N | LYS | 591 | −13.032 | 5.336 | −0.920 | 0.00 | 0.00 | −0.650 | 9.00 | −17.40 |
| ATOM | 4169 | HN | LYS | 591 | −12.436 | 4.502 | −1.025 | 0.00 | 0.00 | 0.439 | 0.00 | 0.00 |
| ATOM | 4170 | CA | LYS | 591 | −12.875 | 6.228 | 0.189 | 0.00 | 0.00 | 0.158 | 9.40 | 4.00 |
| ATOM | 4171 | HA | LYS | 591 | −13.859 | 6.359 | 0.639 | 0.00 | 0.00 | 0.053 | 0.00 | 0.00 |
| ATOM | 4172 | C | LYS | 591 | −12.336 | 7.640 | −0.074 | 0.00 | 0.00 | 0.396 | 9.82 | 4.00 |
| ATOM | 4173 | O | LYS | 591 | −13.114 | 8.591 | −0.072 | 0.00 | 0.00 | −0.396 | 8.17 | −17.40 |
| ATOM | 4174 | CB | LYS | 591 | −12.051 | 5.574 | 1.307 | 0.00 | 0.00 | −0.106 | 12.77 | 4.00 |
| ATOM | 4175 | HB1 | LYS | 591 | −11.842 | 6.256 | 2.131 | 0.00 | 0.00 | 0.053 | 0.00 | 0.00 |
| ATOM | 4176 | HB2 | LYS | 591 | −11.082 | 5.215 | 0.961 | 0.00 | 0.00 | 0.053 | 0.00 | 0.00 |
| ATOM | 4177 | CG | LYS | 591 | −12.752 | 4.364 | 1.928 | 0.00 | 0.00 | −0.106 | 12.77 | 4.00 |
| ATOM | 4178 | HG1 | LYS | 591 | −12.176 | 3.894 | 2.725 | 0.00 | 0.00 | 0.053 | 0.00 | 0.00 |
| ATOM | 4179 | HG2 | LYS | 591 | −12.955 | 3.569 | 1.211 | 0.00 | 0.00 | 0.053 | 0.00 | 0.00 |
| ATOM | 4180 | CD | LYS | 591 | −14.107 | 4.704 | 2.552 | 0.00 | 0.00 | −0.106 | 12.77 | 4.00 |
| ATOM | 4181 | HD1 | LYS | 591 | −14.798 | 5.191 | 1.864 | 0.00 | 0.00 | 0.053 | 0.00 | 0.00 |
| ATOM | 4182 | HD2 | LYS | 591 | −14.041 | 5.378 | 3.406 | 0.00 | 0.00 | 0.053 | 0.00 | 0.00 |
| ATOM | 4183 | CE | LYS | 591 | −14.869 | 3.485 | 3.074 | 0.00 | 0.00 | 0.099 | 12.77 | 4.00 |
| ATOM | 4184 | HE1 | LYS | 591 | −14.850 | 2.686 | 2.332 | 0.00 | 0.00 | 0.053 | 0.00 | 0.00 |
| ATOM | 4185 | HE2 | LYS | 591 | −15.906 | 3.752 | 3.278 | 0.00 | 0.00 | 0.053 | 0.00 | 0.00 |
| ATOM | 4186 | NZ | LYS | 591 | −14.246 | 2.994 | 4.323 | 0.00 | 0.00 | −0.045 | 13.25 | −39.20 |
| ATOM | 4187 | HZ1 | LYS | 591 | −14.765 | 2.174 | 4.667 | 0.00 | 0.00 | 0.280 | 0.00 | 0.00 |
| ATOM | 4188 | HZ2 | LYS | 591 | −13.268 | 2.728 | 4.138 | 0.00 | 0.00 | 0.280 | 0.00 | 0.00 |
| ATOM | 4189 | HZ3 | LYS | 591 | −14.270 | 3.738 | 5.035 | 0.00 | 0.00 | 0.280 | 0.00 | 0.00 |
| ATOM | 4190 | N | ALA | 592 | −11.008 | 7.827 | −0.322 | 0.00 | 0.00 | −0.650 | 9.00 | −17.40 |
| ATOM | 4191 | HN | ALA | 592 | −10.410 | 7.019 | −0.546 | 0.00 | 0.00 | 0.439 | 0.00 | 0.00 |
| ATOM | 4192 | CA | ALA | 592 | −10.429 | 9.165 | −0.272 | 0.00 | 0.00 | 0.158 | 9.40 | 4.00 |
| ATOM | 4193 | HA | ALA | 592 | −10.924 | 9.682 | 0.550 | 0.00 | 0.00 | 0.053 | 0.00 | 0.00 |
| ATOM | 4194 | C | ALA | 592 | −10.512 | 10.120 | −1.435 | 0.00 | 0.00 | 0.396 | 9.82 | 4.00 |
| ATOM | 4195 | O | ALA | 592 | −11.274 | 11.082 | −1.380 | 0.00 | 0.00 | −0.396 | 8.17 | −17.40 |
| ATOM | 4196 | CB | ALA | 592 | −8.940 | 9.202 | 0.108 | 0.00 | 0.00 | −0.159 | 16.15 | 4.00 |
| ATOM | 4197 | HB1 | ALA | 592 | −8.593 | 10.235 | 0.121 | 0.00 | 0.00 | 0.053 | 0.00 | 0.00 |
| ATOM | 4198 | HB2 | ALA | 592 | −8.806 | 8.762 | 1.096 | 0.00 | 0.00 | 0.053 | 0.00 | 0.00 |
| ATOM | 4199 | HB3 | ALA | 592 | −8.364 | 8.635 | −0.623 | 0.00 | 0.00 | 0.053 | 0.00 | 0.00 |
| ATOM | 4200 | N | ILE | 593 | −9.748 | 9.873 | −2.533 | 0.00 | 0.00 | −0.650 | 9.00 | −17.40 |
| ATOM | 4201 | HN | ILE | 593 | −9.252 | 8.975 | −2.630 | 0.00 | 0.00 | 0.439 | 0.00 | 0.00 |
| ATOM | 4202 | CA | ILE | 593 | −9.637 | 10.885 | −3.567 | 0.00 | 0.00 | 0.158 | 9.40 | 4.00 |
| ATOM | 4203 | HA | ILE | 593 | −10.583 | 11.398 | −3.740 | 0.00 | 0.00 | 0.053 | 0.00 | 0.00 |
| ATOM | 4204 | C | ILE | 593 | −9.210 | 10.296 | −4.894 | 0.00 | 0.00 | 0.396 | 9.82 | 4.00 |
| ATOM | 4205 | O | ILE | 593 | −8.591 | 9.235 | −4.935 | 0.00 | 0.00 | −0.396 | 8.17 | −17.40 |
| ATOM | 4206 | CB | ILE | 593 | −8.631 | 11.952 | −3.188 | 0.00 | 0.00 | −0.053 | 9.40 | 4.00 |
| ATOM | 4207 | HB | ILE | 593 | −7.672 | 11.502 | −2.932 | 0.00 | 0.00 | 0.053 | 0.00 | 0.00 |
| ATOM | 4208 | CG1 | ILE | 593 | −9.093 | 12.687 | −1.920 | 0.00 | 0.00 | −0.106 | 12.77 | 4.00 |
| ATOM | 4209 | HG11 | ILE | 593 | −8.286 | 13.339 | −1.587 | 0.00 | 0.00 | 0.053 | 0.00 | 0.00 |
| ATOM | 4210 | HG12 | ILE | 593 | −9.324 | 11.944 | −1.156 | 0.00 | 0.00 | 0.053 | 0.00 | 0.00 |
| ATOM | 4211 | CG2 | ILE | 593 | −8.410 | 12.928 | −4.357 | 0.00 | 0.00 | −0.159 | 16.15 | 4.00 |
| ATOM | 4212 | HG21 | ILE | 593 | −7.684 | 13.687 | −4.065 | 0.00 | 0.00 | 0.053 | 0.00 | 0.00 |
| ATOM | 4213 | HG22 | ILE | 593 | −8.035 | 12.381 | −5.222 | 0.00 | 0.00 | 0.053 | 0.00 | 0.00 |
| ATOM | 4214 | HG23 | ILE | 593 | −9.354 | 13.409 | −4.613 | 0.00 | 0.00 | 0.053 | 0.00 | 0.00 |
| ATOM | 4215 | CD1 | ILE | 593 | −10.337 | 13.549 | −2.125 | 0.00 | 0.00 | −0.159 | 16.15 | 4.00 |
| ATOM | 4216 | HD11 | ILE | 593 | −10.601 | 14.035 | −1.186 | 0.00 | 0.00 | 0.053 | 0.00 | 0.00 |
| ATOM | 4217 | HD12 | ILE | 593 | −10.134 | 14.307 | −2.882 | 0.00 | 0.00 | 0.053 | 0.00 | 0.00 |
| ATOM | 4218 | HD13 | ILE | 593 | −11.165 | 12.921 | −2.454 | 0.00 | 0.00 | 0.053 | 0.00 | 0.00 |
| ATOM | 4219 | N | LEU | 594 | −9.565 | 10.979 | −6.016 | 0.00 | 0.00 | −0.650 | 9.00 | −17.40 |
| ATOM | 4220 | HN | LEU | 594 | −10.097 | 11.852 | −5.892 | 0.00 | 0.00 | 0.439 | 0.00 | 0.00 |
| ATOM | 4221 | CA | LEU | 594 | −9.255 | 10.576 | −7.365 | 0.00 | 0.00 | 0.158 | 9.40 | 4.00 |
| ATOM | 4222 | HA | LEU | 594 | −8.317 | 10.021 | −7.343 | 0.00 | 0.00 | 0.053 | 0.00 | 0.00 |
| ATOM | 4223 | C | LEU | 594 | −9.124 | 11.816 | −8.201 | 0.00 | 0.00 | 0.396 | 9.82 | 4.00 |
| ATOM | 4224 | O | LEU | 594 | −9.550 | 12.896 | −7.795 | 0.00 | 0.00 | −0.396 | 8.17 | −17.40 |
| ATOM | 4225 | CB | LEU | 594 | −10.361 | 9.733 | −8.021 | 0.00 | 0.00 | −0.106 | 12.77 | 4.00 |
| ATOM | 4226 | HB1 | LEU | 594 | −11.320 | 10.247 | −8.085 | 0.00 | 0.00 | 0.053 | 0.00 | 0.00 |
| ATOM | 4227 | HB2 | LEU | 594 | −10.571 | 8.803 | −7.492 | 0.00 | 0.00 | 0.053 | 0.00 | 0.00 |
| ATOM | 4228 | CG | LEU | 594 | −10.036 | 9.309 | −9.464 | 0.00 | 0.00 | −0.053 | 9.40 | 4.00 |
| ATOM | 4229 | HG | LEU | 594 | −9.905 | 10.180 | −10.106 | 0.00 | 0.00 | 0.053 | 0.00 | 0.00 |
| ATOM | 4230 | CD1 | LEU | 594 | −8.743 | 8.485 | −9.515 | 0.00 | 0.00 | −0.159 | 16.15 | 4.00 |
| ATOM | 4231 | HD11 | LEU | 594 | −8.535 | 8.198 | −10.546 | 0.00 | 0.00 | 0.053 | 0.00 | 0.00 |
| ATOM | 4232 | HD12 | LEU | 594 | −7.916 | 9.082 | −9.131 | 0.00 | 0.00 | 0.053 | 0.00 | 0.00 |
| ATOM | 4233 | HD13 | LEU | 594 | −8.858 | 7.589 | −8.905 | 0.00 | 0.00 | 0.053 | 0.00 | 0.00 |
| ATOM | 4234 | CD2 | LEU | 594 | −11.226 | 8.588 | −10.120 | 0.00 | 0.00 | −0.159 | 16.15 | 4.00 |
| ATOM | 4235 | HD21 | LEU | 594 | −10.962 | 8.302 | −11.138 | 0.00 | 0.00 | 0.053 | 0.00 | 0.00 |
| ATOM | 4236 | HD22 | LEU | 594 | −11.472 | 7.695 | −9.545 | 0.00 | 0.00 | 0.053 | 0.00 | 0.00 |
| ATOM | 4237 | HD23 | LEU | 594 | −12.088 | 9.255 | −10.142 | 0.00 | 0.00 | 0.053 | 0.00 | 0.00 |
| ATOM | 4238 | N | ASN | 595 | −8.518 | 11.689 | −9.403 | 0.00 | 0.00 | −0.650 | 9.00 | −17.40 |
| ATOM | 4239 | HN | ASN | 595 | −8.178 | 10.763 | −9.701 | 0.00 | 0.00 | 0.439 | 0.00 | 0.00 |
| ATOM | 4240 | CA | ASN | 595 | −8.338 | 12.817 | −10.274 | 0.00 | 0.00 | 0.158 | 9.40 | 4.00 |
| ATOM | 4241 | HA | ASN | 595 | −8.530 | 13.744 | −9.734 | 0.00 | 0.00 | 0.053 | 0.00 | 0.00 |
| ATOM | 4242 | C | ASN | 595 | −9.282 | 12.731 | −11.433 | 0.00 | 0.00 | 0.396 | 9.82 | 4.00 |
| ATOM | 4243 | O | ASN | 595 | −8.970 | 12.120 | −12.455 | 0.00 | 0.00 | −0.396 | 8.17 | −17.40 |
| ATOM | 4244 | CB | ASN | 595 | −6.941 | 12.887 | −10.898 | 0.00 | 0.00 | −0.106 | 12.77 | 4.00 |
| ATOM | 4245 | HB1 | ASN | 595 | −6.838 | 11.958 | −11.459 | 0.00 | 0.00 | 0.053 | 0.00 | 0.00 |
| ATOM | 4246 | HB2 | ASN | 595 | −6.260 | 12.964 | −10.051 | 0.00 | 0.00 | 0.053 | 0.00 | 0.00 |
| ATOM | 4247 | CG | ASN | 595 | −6.967 | 14.124 | −11.771 | 0.00 | 0.00 | 0.396 | 9.82 | 4.00 |
| ATOM | 4248 | OD1 | ASN | 595 | −7.249 | 15.218 | −11.285 | 0.00 | 0.00 | −0.396 | 8.17 | −17.40 |
| ATOM | 4249 | ND2 | ASN | 595 | −6.711 | 13.945 | −13.095 | 0.00 | 0.00 | −0.879 | 13.25 | −17.40 |
| ATOM | 4250 | HD21 | ASN | 595 | −6.479 | 13.007 | −13.453 | 0.00 | 0.00 | 0.439 | 0.00 | 0.00 |
| ATOM | 4251 | HD22 | ASN | 595 | −6.749 | 14.747 | −13.740 | 0.00 | 0.00 | 0.439 | 0.00 | 0.00 |
| ATOM | 4252 | N | GLN | 596 | −10.476 | 13.334 | −11.305 | 0.00 | 0.00 | −0.650 | 9.00 | −17.40 |
| ATOM | 4253 | HN | GLN | 596 | −10.721 | 13.799 | −10.419 | 0.00 | 0.00 | 0.439 | 0.00 | 0.00 |
| ATOM | 4254 | CA | GLN | 596 | −11.412 | 13.335 | −12.391 | 0.00 | 0.00 | 0.158 | 9.40 | 4.00 |
| ATOM | 4255 | HA | GLN | 596 | −11.511 | 12.343 | −12.832 | 0.00 | 0.00 | 0.053 | 0.00 | 0.00 |
| ATOM | 4256 | C | GLN | 596 | −10.985 | 14.272 | −13.482 | 0.00 | 0.00 | 0.396 | 9.82 | 4.00 |
| ATOM | 4257 | O | GLN | 596 | −11.095 | 13.940 | −14.662 | 0.00 | 0.00 | −0.396 | 8.17 | −17.40 |
| ATOM | 4258 | CB | GLN | 596 | −12.840 | 13.716 | −11.963 | 0.00 | 0.00 | −0.106 | 12.77 | 4.00 |
| ATOM | 4259 | HB1 | GLN | 596 | −13.285 | 13.020 | −11.252 | 0.00 | 0.00 | 0.053 | 0.00 | 0.00 |
| ATOM | 4260 | HB2 | GLN | 596 | −13.548 | 13.762 | −12.790 | 0.00 | 0.00 | 0.053 | 0.00 | 0.00 |
| ATOM | 4261 | CG | GLN | 596 | −12.935 | 15.082 | −11.288 | 0.00 | 0.00 | −0.106 | 12.77 | 4.00 |
| ATOM | 4262 | HG1 | GLN | 596 | −13.970 | 15.403 | −11.406 | 0.00 | 0.00 | 0.053 | 0.00 | 0.00 |
| ATOM | 4263 | HG2 | GLN | 596 | −12.231 | 15.726 | −11.815 | 0.00 | 0.00 | 0.053 | 0.00 | 0.00 |
| ATOM | 4264 | CD | GLN | 596 | −12.551 | 14.856 | −9.833 | 0.00 | 0.00 | 0.396 | 9.82 | 4.00 |
| ATOM | 4265 | OE1 | GLN | 596 | −12.936 | 13.847 | −9.244 | 0.00 | 0.00 | −0.396 | 8.17 | −17.40 |
| ATOM | 4266 | NE2 | GLN | 596 | −11.771 | 15.800 | −9.243 | 0.00 | 0.00 | −0.879 | 13.25 | −17.40 |
| ATOM | 4267 | HE21 | GLN | 596 | −11.473 | 16.629 | −9.777 | 0.00 | 0.00 | 0.439 | 0.00 | 0.00 |
| ATOM | 4268 | HE22 | GLN | 596 | −11.478 | 15.686 | −8.262 | 0.00 | 0.00 | 0.439 | 0.00 | 0.00 |
| ATOM | 4269 | N | LYS | 597 | −10.459 | 15.461 | −13.125 | 0.00 | 0.00 | −0.650 | 9.00 | −17.40 |
| ATOM | 4270 | HN | LYS | 597 | −10.247 | 15.652 | −12.135 | 0.00 | 0.00 | 0.439 | 0.00 | 0.00 |
| ATOM | 4271 | CA | LYS | 597 | −10.194 | 16.462 | −14.120 | 0.00 | 0.00 | 0.158 | 9.40 | 4.00 |
| ATOM | 4272 | HA | LYS | 597 | −11.124 | 16.748 | −14.611 | 0.00 | 0.00 | 0.053 | 0.00 | 0.00 |
| ATOM | 4273 | C | LYS | 597 | −9.245 | 15.921 | −15.141 | 0.00 | 0.00 | 0.396 | 9.82 | 4.00 |
| ATOM | 4274 | O | LYS | 597 | −8.352 | 15.133 | −14.834 | 0.00 | 0.00 | −0.396 | 8.17 | −17.40 |
| ATOM | 4275 | CB | LYS | 597 | −9.586 | 17.760 | −13.562 | 0.00 | 0.00 | −0.106 | 12.77 | 4.00 |
| ATOM | 4276 | HB1 | LYS | 597 | −8.502 | 17.710 | −13.462 | 0.00 | 0.00 | 0.053 | 0.00 | 0.00 |
| ATOM | 4277 | HB2 | LYS | 597 | −9.966 | 18.014 | −12.573 | 0.00 | 0.00 | 0.053 | 0.00 | 0.00 |
| ATOM | 4278 | CG | LYS | 597 | −9.870 | 18.976 | −14.445 | 0.00 | 0.00 | −0.106 | 12.77 | 4.00 |
| ATOM | 4279 | HG1 | LYS | 597 | −9.798 | 18.757 | −15.510 | 0.00 | 0.00 | 0.053 | 0.00 | 0.00 |
| ATOM | 4280 | HG2 | LYS | 597 | −9.182 | 19.802 | −14.268 | 0.00 | 0.00 | 0.053 | 0.00 | 0.00 |
| ATOM | 4281 | CD | LYS | 597 | −11.274 | 19.556 | −14.236 | 0.00 | 0.00 | −0.106 | 12.77 | 4.00 |
| ATOM | 4282 | HD1 | LYS | 597 | −11.567 | 20.079 | −15.146 | 0.00 | 0.00 | 0.053 | 0.00 | 0.00 |
| ATOM | 4283 | HD2 | LYS | 597 | −11.238 | 20.245 | −13.392 | 0.00 | 0.00 | 0.053 | 0.00 | 0.00 |
| ATOM | 4284 | CE | LYS | 597 | −12.350 | 18.509 | −13.935 | 0.00 | 0.00 | 0.099 | 12.77 | 4.00 |
| ATOM | 4285 | HE1 | LYS | 597 | −13.272 | 18.993 | −13.612 | 0.00 | 0.00 | 0.053 | 0.00 | 0.00 |
| ATOM | 4286 | HE2 | LYS | 597 | −12.016 | 17.837 | −13.144 | 0.00 | 0.00 | 0.053 | 0.00 | 0.00 |
| ATOM | 4287 | NZ | LYS | 597 | −12.640 | 17.705 | −15.143 | 0.00 | 0.00 | −0.045 | 13.25 | −39.20 |
| ATOM | 4288 | HZ1 | LYS | 597 | −13.365 | 17.006 | −14.925 | 0.00 | 0.00 | 0.280 | 0.00 | 0.00 |
| ATOM | 4289 | HZ2 | LYS | 597 | −12.975 | 18.323 | −15.895 | 0.00 | 0.00 | 0.280 | 0.00 | 0.00 |
| ATOM | 4290 | HZ3 | LYS | 597 | −11.782 | 17.225 | −15.451 | 0.00 | 0.00 | 0.280 | 0.00 | 0.00 |
| ATOM | 4291 | N | ILE | 598 | −9.434 | 16.338 | −16.408 | 0.00 | 0.00 | −0.650 | 9.00 | −17.40 |
| ATOM | 4292 | HN | ILE | 598 | −10.208 | 16.985 | −16.617 | 0.00 | 0.00 | 0.439 | 0.00 | 0.00 |
| ATOM | 4293 | CA | ILE | 598 | −8.579 | 15.899 | −17.471 | 0.00 | 0.00 | 0.158 | 9.40 | 4.00 |
| ATOM | 4294 | HA | ILE | 598 | −7.861 | 15.224 | −17.005 | 0.00 | 0.00 | 0.053 | 0.00 | 0.00 |
| ATOM | 4295 | C | ILE | 598 | −7.951 | 17.145 | −18.016 | 0.00 | 0.00 | 0.396 | 9.82 | 4.00 |
| ATOM | 4296 | O | ILE | 598 | −8.616 | 18.174 | −18.109 | 0.00 | 0.00 | −0.396 | 8.17 | −17.40 |
| ATOM | 4297 | CB | ILE | 598 | −9.350 | 15.253 | −18.595 | 0.00 | 0.00 | −0.053 | 9.40 | 4.00 |
| ATOM | 4298 | HB | ILE | 598 | −8.627 | 14.876 | −19.319 | 0.00 | 0.00 | 0.053 | 0.00 | 0.00 |
| ATOM | 4299 | CG1 | ILE | 598 | −10.238 | 16.281 | −19.316 | 0.00 | 0.00 | −0.106 | 12.77 | 4.00 |
| ATOM | 4300 | HG11 | ILE | 598 | −9.712 | 17.202 | −19.567 | 0.00 | 0.00 | 0.053 | 0.00 | 0.00 |
| ATOM | 4301 | HG12 | ILE | 598 | −11.102 | 16.590 | −18.727 | 0.00 | 0.00 | 0.053 | 0.00 | 0.00 |
| ATOM | 4302 | CG2 | ILE | 598 | −10.185 | 14.105 | −17.991 | 0.00 | 0.00 | −0.159 | 16.15 | 4.00 |
| ATOM | 4303 | HG21 | ILE | 598 | −10.756 | 13.616 | −18.780 | 0.00 | 0.00 | 0.053 | 0.00 | 0.00 |
| ATOM | 4304 | HG22 | ILE | 598 | −9.520 | 13.380 | −17.521 | 0.00 | 0.00 | 0.053 | 0.00 | 0.00 |
| ATOM | 4305 | HG23 | ILE | 598 | −10.869 | 14.507 | −17.244 | 0.00 | 0.00 | 0.053 | 0.00 | 0.00 |
| ATOM | 4306 | CD1 | ILE | 598 | −10.808 | 15.763 | −20.638 | 0.00 | 0.00 | −0.159 | 16.15 | 4.00 |
| ATOM | 4307 | HD11 | ILE | 598 | −11.425 | 16.536 | −21.096 | 0.00 | 0.00 | 0.053 | 0.00 | 0.00 |
| ATOM | 4308 | HD12 | ILE | 598 | −9.990 | 15.506 | −21.311 | 0.00 | 0.00 | 0.053 | 0.00 | 0.00 |
| ATOM | 4309 | HD13 | ILE | 598 | −11.416 | 14.878 | −20.450 | 0.00 | 0.00 | 0.053 | 0.00 | 0.00 |
| ATOM | 4310 | N | GLU | 599 | −6.650 | 17.110 | −18.395 | 0.00 | 0.00 | −0.650 | 9.00 | −17.40 |
| ATOM | 4311 | HN | GLU | 599 | −6.100 | 16.245 | −18.297 | 0.00 | 0.00 | 0.439 | 0.00 | 0.00 |
| ATOM | 4312 | CA | GLU | 599 | −6.046 | 18.304 | −18.939 | 0.00 | 0.00 | 0.158 | 9.40 | 4.00 |
| ATOM | 4313 | HA | GLU | 599 | −6.675 | 19.184 | −18.808 | 0.00 | 0.00 | 0.053 | 0.00 | 0.00 |
| ATOM | 4314 | C | GLU | 599 | −5.775 | 18.188 | −20.428 | 0.00 | 0.00 | 0.396 | 9.82 | 4.00 |
| ATOM | 4315 | O | GLU | 599 | −4.644 | 17.982 | −20.851 | 0.00 | 0.00 | −0.396 | 8.17 | −17.40 |
| ATOM | 4316 | CB | GLU | 599 | −4.745 | 18.701 | −18.199 | 0.00 | 0.00 | −0.106 | 12.77 | 4.00 |
| ATOM | 4317 | HB1 | GLU | 599 | −5.024 | 19.276 | −17.316 | 0.00 | 0.00 | 0.053 | 0.00 | 0.00 |
| ATOM | 4318 | HB2 | GLU | 599 | −4.141 | 19.301 | −18.880 | 0.00 | 0.00 | 0.053 | 0.00 | 0.00 |
| ATOM | 4319 | CG | GLU | 599 | −3.863 | 17.535 | −17.720 | 0.00 | 0.00 | −0.336 | 12.77 | 4.00 |
| ATOM | 4320 | HG1 | GLU | 599 | −4.453 | 16.765 | −17.222 | 0.00 | 0.00 | 0.053 | 0.00 | 0.00 |
| ATOM | 4321 | HG2 | GLU | 599 | −3.104 | 17.871 | −17.013 | 0.00 | 0.00 | 0.053 | 0.00 | 0.00 |
| ATOM | 4322 | CD | GLU | 599 | −3.138 | 16.870 | −18.880 | 0.00 | 0.00 | 0.297 | 9.82 | 4.00 |
| ATOM | 4323 | OE1 | GLU | 599 | −2.363 | 17.575 | −19.579 | 0.00 | 0.00 | −0.534 | 8.17 | −18.95 |
| ATOM | 4324 | OE2 | GLU | 599 | −3.364 | 15.648 | −19.094 | 0.00 | 0.00 | −0.534 | 8.17 | −18.95 |
| ATOM | 4325 | N | PRO | 600 | −6.771 | 18.311 | −21.270 | 0.00 | 0.00 | −0.422 | 9.00 | −17.40 |
| ATOM | 4326 | CA | PRO | 600 | −6.485 | 18.232 | −22.685 | 0.00 | 0.00 | 0.158 | 9.40 | 4.00 |
| ATOM | 4327 | HA | PRO | 600 | −5.414 | 18.071 | −22.809 | 0.00 | 0.00 | 0.053 | 0.00 | 0.00 |
| ATOM | 4328 | CD | PRO | 600 | −8.024 | 17.645 | −20.973 | 0.00 | 0.00 | 0.105 | 12.77 | 4.00 |
| ATOM | 4329 | HD1 | PRO | 600 | −8.658 | 18.385 | −20.484 | 0.00 | 0.00 | 0.053 | 0.00 | 0.00 |
| ATOM | 4330 | HD2 | PRO | 600 | −7.780 | 16.810 | −20.317 | 0.00 | 0.00 | 0.053 | 0.00 | 0.00 |
| ATOM | 4331 | C | PRO | 600 | −6.903 | 19.524 | −23.322 | 0.00 | 0.00 | 0.396 | 9.82 | 4.00 |
| ATOM | 4332 | O | PRO | 600 | −7.183 | 20.480 | −22.604 | 0.00 | 0.00 | −0.396 | 8.17 | −17.40 |
| ATOM | 4333 | CB | PRO | 600 | −7.361 | 17.108 | −23.219 | 0.00 | 0.00 | −0.106 | 12.77 | 4.00 |
| ATOM | 4334 | HB1 | PRO | 600 | −6.856 | 16.145 | −23.142 | 0.00 | 0.00 | 0.053 | 0.00 | 0.00 |
| ATOM | 4335 | HB2 | PRO | 600 | −7.611 | 17.268 | −24.268 | 0.00 | 0.00 | 0.053 | 0.00 | 0.00 |
| ATOM | 4336 | CG | PRO | 600 | −8.597 | 17.183 | −22.316 | 0.00 | 0.00 | −0.106 | 12.77 | 4.00 |
| ATOM | 4337 | HG1 | PRO | 600 | −9.083 | 16.211 | −22.236 | 0.00 | 0.00 | 0.053 | 0.00 | 0.00 |
| ATOM | 4338 | HG2 | PRO | 600 | −9.327 | 17.890 | −22.710 | 0.00 | 0.00 | 0.053 | 0.00 | 0.00 |
| ATOM | 4339 | N | TYR | 601 | −6.944 | 19.556 | −24.678 | 0.00 | 0.00 | −0.650 | 9.00 | −17.40 |
| ATOM | 4340 | HN | TYR | 601 | −6.493 | 18.801 | −25.215 | 0.00 | 0.00 | 0.439 | 0.00 | 0.00 |
| ATOM | 4341 | CA | TYR | 601 | −7.604 | 20.623 | −25.381 | 0.00 | 0.00 | 0.158 | 9.40 | 4.00 |
| ATOM | 4342 | HA | TYR | 601 | −8.596 | 20.715 | −24.939 | 0.00 | 0.00 | 0.053 | 0.00 | 0.00 |
| ATOM | 4343 | C | TYR | 601 | −7.658 | 20.210 | −26.824 | 0.00 | 0.00 | 0.396 | 9.82 | 4.00 |
| ATOM | 4344 | O | TYR | 601 | −7.016 | 19.230 | −27.201 | 0.00 | 0.00 | −0.396 | 8.17 | −17.40 |
| ATOM | 4345 | CB | TYR | 601 | −6.967 | 22.015 | −25.273 | 0.00 | 0.00 | −0.106 | 12.77 | 4.00 |
| ATOM | 4346 | HB1 | TYR | 601 | −6.222 | 22.175 | −26.053 | 0.00 | 0.00 | 0.053 | 0.00 | 0.00 |
| ATOM | 4347 | HB2 | TYR | 601 | −6.469 | 22.153 | −24.313 | 0.00 | 0.00 | 0.053 | 0.00 | 0.00 |
| ATOM | 4348 | CG | TYR | 601 | −8.156 | 22.900 | −25.430 | 0.00 | 0.00 | 0.000 | 7.26 | 0.60 |
| ATOM | 4349 | CD1 | TYR | 601 | −9.114 | 22.837 | −24.447 | 0.00 | 0.00 | −0.127 | 10.80 | 0.60 |
| ATOM | 4350 | HD1 | TYR | 601 | −8.974 | 22.156 | −23.607 | 0.00 | 0.00 | 0.127 | 0.00 | 0.00 |
| ATOM | 4351 | CD2 | TYR | 601 | −8.336 | 23.768 | −26.483 | 0.00 | 0.00 | −0.127 | 10.80 | 0.60 |
| ATOM | 4352 | HD2 | TYR | 601 | −7.583 | 23.835 | −27.268 | 0.00 | 0.00 | 0.127 | 0.00 | 0.00 |
| ATOM | 4353 | CE1 | TYR | 601 | −10.242 | 23.613 | −24.502 | 0.00 | 0.00 | −0.127 | 10.80 | 0.60 |
| ATOM | 4354 | HE1 | TYR | 601 | −10.993 | 23.548 | −23.714 | 0.00 | 0.00 | 0.127 | 0.00 | 0.00 |
| ATOM | 4355 | CE2 | TYR | 601 | −9.467 | 24.552 | −26.545 | 0.00 | 0.00 | −0.127 | 10.80 | 0.60 |
| ATOM | 4356 | HE2 | TYR | 601 | −9.608 | 25.238 | −27.380 | 0.00 | 0.00 | 0.127 | 0.00 | 0.00 |
| ATOM | 4357 | CZ | TYR | 601 | −10.419 | 24.472 | −25.556 | 0.00 | 0.00 | 0.027 | 7.26 | 0.60 |
| ATOM | 4358 | OH | TYR | 601 | −11.578 | 25.272 | −25.616 | 0.00 | 0.00 | −0.451 | 10.94 | −17.40 |
| ATOM | 4359 | HH | TYR | 601 | −11.310 | 26.266 | −25.618 | 0.00 | 0.00 | 0.424 | 0.00 | 0.00 |
| ATOM | 4360 | N | MET | 602 | −8.435 | 20.912 | −27.676 | 0.00 | 0.00 | −0.650 | 9.00 | −17.40 |
| ATOM | 4361 | HN | MET | 602 | −8.952 | 21.749 | −27.370 | 0.00 | 0.00 | 0.439 | 0.00 | 0.00 |
| ATOM | 4362 | CA | MET | 602 | −8.507 | 20.435 | −29.032 | 0.00 | 0.00 | 0.158 | 9.40 | 4.00 |
| ATOM | 4363 | HA | MET | 602 | −8.819 | 19.391 | −28.994 | 0.00 | 0.00 | 0.053 | 0.00 | 0.00 |
| ATOM | 4364 | C | MET | 602 | −7.143 | 20.578 | −29.629 | 0.00 | 0.00 | 0.396 | 9.82 | 4.00 |
| ATOM | 4365 | O | MET | 602 | −6.659 | 19.639 | −30.256 | 0.00 | 0.00 | −0.396 | 8.17 | −17.40 |
| ATOM | 4366 | CB | MET | 602 | −9.411 | 21.216 | −30.014 | 0.00 | 0.00 | −0.106 | 12.77 | 4.00 |
| ATOM | 4367 | HB1 | MET | 602 | −9.632 | 20.557 | −30.853 | 0.00 | 0.00 | 0.053 | 0.00 | 0.00 |
| ATOM | 4368 | HB2 | MET | 602 | −8.859 | 22.098 | −30.339 | 0.00 | 0.00 | 0.053 | 0.00 | 0.00 |
| ATOM | 4369 | CG | MET | 602 | −10.747 | 21.709 | −29.484 | 0.00 | 0.00 | −0.041 | 12.77 | 4.00 |
| ATOM | 4370 | HG1 | MET | 602 | −11.275 | 22.195 | −30.304 | 0.00 | 0.00 | 0.053 | 0.00 | 0.00 |
| ATOM | 4371 | HG2 | MET | 602 | −10.549 | 22.414 | −28.676 | 0.00 | 0.00 | 0.053 | 0.00 | 0.00 |
| ATOM | 4372 | SD | MET | 602 | −11.843 | 20.436 | −28.824 | 0.00 | 0.00 | −0.130 | 16.39 | −6.40 |
| ATOM | 4373 | CE | MET | 602 | −13.175 | 21.655 | −28.637 | 0.00 | 0.00 | −0.094 | 16.15 | 4.00 |
| ATOM | 4374 | HE1 | MET | 602 | −14.059 | 21.168 | −28.225 | 0.00 | 0.00 | 0.053 | 0.00 | 0.00 |
| ATOM | 4375 | HE2 | MET | 602 | −12.850 | 22.448 | −27.963 | 0.00 | 0.00 | 0.053 | 0.00 | 0.00 |
| ATOM | 4376 | HE3 | MET | 602 | −13.417 | 22.082 | −29.610 | 0.00 | 0.00 | 0.053 | 0.00 | 0.00 |
| ATOM | 4377 | N | PRO | 603 | −6.478 | 21.707 | −29.466 | 0.00 | 0.00 | −0.422 | 9.00 | −17.40 |
| ATOM | 4378 | CA | PRO | 603 | −5.191 | 21.898 | −30.092 | 0.00 | 0.00 | 0.158 | 9.40 | 4.00 |
| ATOM | 4379 | HA | PRO | 603 | −5.195 | 21.466 | −31.093 | 0.00 | 0.00 | 0.053 | 0.00 | 0.00 |
| ATOM | 4380 | CD | PRO | 603 | −7.122 | 22.983 | −29.204 | 0.00 | 0.00 | 0.105 | 12.77 | 4.00 |
| ATOM | 4381 | HD1 | PRO | 603 | −7.563 | 22.875 | −28.213 | 0.00 | 0.00 | 0.053 | 0.00 | 0.00 |
| ATOM | 4382 | HD2 | PRO | 603 | −7.858 | 23.093 | −30.001 | 0.00 | 0.00 | 0.053 | 0.00 | 0.00 |
| ATOM | 4383 | C | PRO | 603 | −4.130 | 21.237 | −29.286 | 0.00 | 0.00 | 0.396 | 9.82 | 4.00 |
| ATOM | 4384 | O | PRO | 603 | −4.320 | 21.095 | −28.078 | 0.00 | 0.00 | −0.396 | 8.17 | −17.40 |
| ATOM | 4385 | CB | PRO | 603 | −4.991 | 23.405 | −30.256 | 0.00 | 0.00 | −0.106 | 12.77 | 4.00 |
| ATOM | 4386 | HB1 | PRO | 603 | −5.198 | 23.717 | −31.280 | 0.00 | 0.00 | 0.053 | 0.00 | 0.00 |
| ATOM | 4387 | HB2 | PRO | 603 | −3.966 | 23.691 | −30.019 | 0.00 | 0.00 | 0.053 | 0.00 | 0.00 |
| ATOM | 4388 | CG | PRO | 603 | −5.993 | 24.011 | −29.265 | 0.00 | 0.00 | −0.106 | 12.77 | 4.00 |
| ATOM | 4389 | HG1 | PRO | 603 | −6.275 | 24.960 | −29.721 | 0.00 | 0.00 | 0.053 | 0.00 | 0.00 |
| ATOM | 4390 | HG2 | PRO | 603 | −5.424 | 24.108 | −28.340 | 0.00 | 0.00 | 0.053 | 0.00 | 0.00 |
| ATOM | 4391 | N | TYR | 604 | −2.986 | 20.890 | −29.924 | 0.00 | 0.00 | −0.650 | 9.00 | −17.40 |
| ATOM | 4392 | HN | TYR | 604 | −2.865 | 21.233 | −30.888 | 0.00 | 0.00 | 0.439 | 0.00 | 0.00 |
| ATOM | 4393 | CA | TYR | 604 | −1.921 | 20.084 | −29.377 | 0.00 | 0.00 | 0.158 | 9.40 | 4.00 |
| ATOM | 4394 | HA | TYR | 604 | −1.341 | 19.619 | −30.174 | 0.00 | 0.00 | 0.053 | 0.00 | 0.00 |
| ATOM | 4395 | C | TYR | 604 | −2.480 | 18.982 | −28.493 | 0.00 | 0.00 | 0.396 | 9.82 | 4.00 |
| ATOM | 4396 | O | TYR | 604 | −3.676 | 18.691 | −28.475 | 0.00 | 0.00 | −0.396 | 8.17 | −17.40 |
| ATOM | 4397 | CB | TYR | 604 | −0.782 | 20.890 | −28.690 | 0.00 | 0.00 | −0.106 | 12.77 | 4.00 |
| ATOM | 4398 | HB1 | TYR | 604 | −0.096 | 20.250 | −28.135 | 0.00 | 0.00 | 0.053 | 0.00 | 0.00 |
| ATOM | 4399 | HB2 | TYR | 604 | −1.162 | 21.622 | −27.978 | 0.00 | 0.00 | 0.053 | 0.00 | 0.00 |
| ATOM | 4400 | CG | TYR | 604 | 0.062 | 21.660 | −29.684 | 0.00 | 0.00 | 0.000 | 7.26 | 0.60 |
| ATOM | 4401 | CD1 | TYR | 604 | −0.365 | 22.870 | −30.184 | 0.00 | 0.00 | −0.127 | 10.80 | 0.60 |
| ATOM | 4402 | HD1 | TYR | 604 | −1.329 | 23.265 | −29.865 | 0.00 | 0.00 | 0.127 | 0.00 | 0.00 |
| ATOM | 4403 | CD2 | TYR | 604 | 1.299 | 21.205 | −30.101 | 0.00 | 0.00 | −0.127 | 10.80 | 0.60 |
| ATOM | 4404 | HD2 | TYR | 604 | 1.676 | 20.258 | −29.715 | 0.00 | 0.00 | 0.127 | 0.00 | 0.00 |
| ATOM | 4405 | CE1 | TYR | 604 | 0.395 | 23.595 | −31.078 | 0.00 | 0.00 | −0.127 | 10.80 | 0.60 |
| ATOM | 4406 | HE1 | TYR | 604 | 0.024 | 24.546 | −31.460 | 0.00 | 0.00 | 0.127 | 0.00 | 0.00 |
| ATOM | 4407 | CE2 | TYR | 604 | 2.069 | 21.923 | −30.996 | 0.00 | 0.00 | −0.127 | 10.80 | 0.60 |
| ATOM | 4408 | HE2 | TYR | 604 | 3.037 | 21.536 | −31.312 | 0.00 | 0.00 | 0.127 | 0.00 | 0.00 |
| ATOM | 4409 | CZ | TYR | 604 | 1.618 | 23.123 | −31.488 | 0.00 | 0.00 | 0.027 | 7.26 | 0.60 |
| ATOM | 4410 | OH | TYR | 604 | 2.396 | 23.865 | −32.403 | 0.00 | 0.00 | −0.451 | 10.94 | −17.40 |
| ATOM | 4411 | HH | TYR | 604 | 3.096 | 24.419 | −31.889 | 0.00 | 0.00 | 0.424 | 0.00 | 0.00 |
| ATOM | 4412 | N | GLU | 605 | −1.638 | 18.238 | −27.760 | 0.00 | 0.00 | −0.650 | 9.00 | −17.40 |
| ATOM | 4413 | HN | GLU | 605 | −0.636 | 18.449 | −27.654 | 0.00 | 0.00 | 0.439 | 0.00 | 0.00 |
| ATOM | 4414 | CA | GLU | 605 | −2.284 | 17.114 | −27.138 | 0.00 | 0.00 | 0.158 | 9.40 | 4.00 |
| ATOM | 4415 | HA | GLU | 605 | −3.359 | 17.271 | −27.051 | 0.00 | 0.00 | 0.053 | 0.00 | 0.00 |
| ATOM | 4416 | C | GLU | 605 | −1.752 | 16.871 | −25.758 | 0.00 | 0.00 | 0.396 | 9.82 | 4.00 |
| ATOM | 4417 | O | GLU | 605 | −2.509 | 16.847 | −24.790 | 0.00 | 0.00 | −0.396 | 8.17 | −17.40 |
| ATOM | 4418 | CB | GLU | 605 | −2.124 | 15.849 | −28.007 | 0.00 | 0.00 | −0.106 | 12.77 | 4.00 |
| ATOM | 4419 | HB1 | GLU | 605 | −2.627 | 15.932 | −28.970 | 0.00 | 0.00 | 0.053 | 0.00 | 0.00 |
| ATOM | 4420 | HB2 | GLU | 605 | −2.531 | 14.956 | −27.532 | 0.00 | 0.00 | 0.053 | 0.00 | 0.00 |
| ATOM | 4421 | CG | GLU | 605 | −0.663 | 15.528 | −28.323 | 0.00 | 0.00 | −0.336 | 12.77 | 4.00 |
| ATOM | 4422 | HG1 | GLU | 605 | −0.318 | 14.790 | −27.599 | 0.00 | 0.00 | 0.053 | 0.00 | 0.00 |
| ATOM | 4423 | HG2 | GLU | 605 | −0.092 | 16.453 | −28.241 | 0.00 | 0.00 | 0.053 | 0.00 | 0.00 |
| ATOM | 4424 | CD | GLU | 605 | −0.602 | 14.973 | −29.738 | 0.00 | 0.00 | 0.297 | 9.82 | 4.00 |
| ATOM | 4425 | OE1 | GLU | 605 | −1.684 | 14.693 | −30.315 | 0.00 | 0.00 | −0.534 | 8.17 | −18.95 |
| ATOM | 4426 | OE2 | GLU | 605 | 0.536 | 14.845 | −30.266 | 0.00 | 0.00 | −0.534 | 8.17 | −18.95 |
| ATOM | 4427 | N | PHE | 606 | −0.428 | 16.686 | −25.641 | 0.00 | 0.00 | −0.650 | 9.00 | −17.40 |
| ATOM | 4428 | HN | PHE | 606 | 0.138 | 16.807 | −26.493 | 0.00 | 0.00 | 0.439 | 0.00 | 0.00 |
| ATOM | 4429 | CA | PHE | 606 | 0.274 | 16.335 | −24.438 | 0.00 | 0.00 | 0.158 | 9.40 | 4.00 |
| ATOM | 4430 | HA | PHE | 606 | −0.046 | 15.335 | −24.147 | 0.00 | 0.00 | 0.053 | 0.00 | 0.00 |
| ATOM | 4431 | C | PHE | 606 | 0.061 | 17.206 | −23.272 | 0.00 | 0.00 | 0.396 | 9.82 | 4.00 |
| ATOM | 4432 | O | PHE | 606 | −0.442 | 16.739 | −22.253 | 0.00 | 0.00 | −0.396 | 8.17 | −17.40 |
| ATOM | 4433 | CB | PHE | 606 | 1.770 | 16.317 | −24.712 | 0.00 | 0.00 | −0.106 | 12.77 | 4.00 |
| ATOM | 4434 | HB1 | PHE | 606 | 2.320 | 16.927 | −23.996 | 0.00 | 0.00 | 0.053 | 0.00 | 0.00 |
| ATOM | 4435 | HB2 | PHE | 606 | 2.004 | 16.701 | −25.705 | 0.00 | 0.00 | 0.053 | 0.00 | 0.00 |
| ATOM | 4436 | CG | PHE | 606 | 2.044 | 14.893 | −24.574 | 0.00 | 0.00 | 0.000 | 7.26 | 0.60 |
| ATOM | 4437 | CD1 | PHE | 606 | 1.312 | 13.956 | −25.265 | 0.00 | 0.00 | −0.127 | 10.80 | 0.60 |
| ATOM | 4438 | HD1 | PHE | 606 | 0.522 | 14.265 | −25.950 | 0.00 | 0.00 | 0.127 | 0.00 | 0.00 |
| ATOM | 4439 | CD2 | PHE | 606 | 3.051 | 14.547 | −23.717 | 0.00 | 0.00 | −0.127 | 10.80 | 0.60 |
| ATOM | 4440 | HD2 | PHE | 606 | 3.616 | 15.316 | −23.189 | 0.00 | 0.00 | 0.127 | 0.00 | 0.00 |
| ATOM | 4441 | CE1 | PHE | 606 | 1.591 | 12.635 | −25.079 | 0.00 | 0.00 | −0.127 | 10.80 | 0.60 |
| ATOM | 4442 | HE1 | PHE | 606 | 1.033 | 11.859 | −25.604 | 0.00 | 0.00 | 0.127 | 0.00 | 0.00 |
| ATOM | 4443 | CE2 | PHE | 606 | 3.338 | 13.234 | −23.532 | 0.00 | 0.00 | −0.127 | 10.80 | 0.60 |
| ATOM | 4444 | HE2 | PHE | 606 | 4.138 | 12.922 | −22.861 | 0.00 | 0.00 | 0.127 | 0.00 | 0.00 |
| ATOM | 4445 | CZ | PHE | 606 | 2.596 | 12.321 | −24.210 | 0.00 | 0.00 | −0.127 | 10.80 | 0.60 |
| ATOM | 4446 | HZ | PHE | 606 | 2.818 | 11.266 | −24.050 | 0.00 | 0.00 | 0.127 | 0.00 | 0.00 |
| ATOM | 4447 | N | THR | 607 | 0.430 | 18.484 | −23.379 | 0.00 | 0.00 | −0.650 | 9.00 | −17.40 |
| ATOM | 4448 | HN | THR | 607 | 0.824 | 18.843 | −24.260 | 0.00 | 0.00 | 0.439 | 0.00 | 0.00 |
| ATOM | 4449 | CA | THR | 607 | 0.270 | 19.343 | −22.254 | 0.00 | 0.00 | 0.158 | 9.40 | 4.00 |
| ATOM | 4450 | HA | THR | 607 | −0.803 | 19.507 | −22.150 | 0.00 | 0.00 | 0.053 | 0.00 | 0.00 |
| ATOM | 4451 | C | THR | 607 | 1.011 | 20.589 | −22.580 | 0.00 | 0.00 | 0.396 | 9.82 | 4.00 |
| ATOM | 4452 | O | THR | 607 | 1.902 | 20.589 | −23.430 | 0.00 | 0.00 | −0.396 | 8.17 | −17.40 |
| ATOM | 4453 | CB | THR | 607 | 0.807 | 18.789 | −20.964 | 0.00 | 0.00 | 0.060 | 9.40 | 4.00 |
| ATOM | 4454 | HB | THR | 607 | 0.299 | 17.851 | −20.740 | 0.00 | 0.00 | 0.053 | 0.00 | 0.00 |
| ATOM | 4455 | OG1 | THR | 607 | 0.528 | 19.673 | −19.892 | 0.00 | 0.00 | −0.537 | 11.04 | −17.40 |
| ATOM | 4456 | HG1 | THR | 607 | 1.066 | 20.543 | −20.015 | 0.00 | 0.00 | 0.424 | 0.00 | 0.00 |
| ATOM | 4457 | CG2 | THR | 607 | 2.314 | 18.543 | −21.112 | 0.00 | 0.00 | −0.159 | 16.15 | 4.00 |
| ATOM | 4458 | HG21 | THR | 607 | 2.710 | 18.141 | −20.180 | 0.00 | 0.00 | 0.053 | 0.00 | 0.00 |
| ATOM | 4459 | HG22 | THR | 607 | 2.489 | 17.830 | −21.918 | 0.00 | 0.00 | 0.053 | 0.00 | 0.00 |
| ATOM | 4460 | HG23 | THR | 607 | 2.815 | 19.483 | −21.344 | 0.00 | 0.00 | 0.053 | 0.00 | 0.00 |
| ATOM | 4461 | N | CYS | 608 | 0.650 | 21.691 | −21.903 | 0.00 | 0.00 | −0.650 | 9.00 | −17.40 |
| ATOM | 4462 | HN | CYS | 608 | −0.030 | 21.623 | −21.132 | 0.00 | 0.00 | 0.439 | 0.00 | 0.00 |
| ATOM | 4463 | CA | CYS | 608 | 1.212 | 22.962 | −22.254 | 0.00 | 0.00 | 0.158 | 9.40 | 4.00 |
| ATOM | 4464 | HA | CYS | 608 | 1.039 | 23.209 | −23.302 | 0.00 | 0.00 | 0.053 | 0.00 | 0.00 |
| ATOM | 4465 | C | CYS | 608 | 2.696 | 22.998 | −22.037 | 0.00 | 0.00 | 0.396 | 9.82 | 4.00 |
| ATOM | 4466 | O | CYS | 608 | 3.456 | 23.301 | −22.954 | 0.00 | 0.00 | −0.396 | 8.17 | −17.40 |
| ATOM | 4467 | CB | CYS | 608 | 0.575 | 24.142 | −21.483 | 0.00 | 0.00 | −0.041 | 12.77 | 4.00 |
| ATOM | 4468 | HB1 | CYS | 608 | −0.502 | 24.226 | −21.630 | 0.00 | 0.00 | 0.053 | 0.00 | 0.00 |
| ATOM | 4469 | HB2 | CYS | 608 | 0.978 | 25.114 | −21.767 | 0.00 | 0.00 | 0.053 | 0.00 | 0.00 |
| ATOM | 4470 | SG | CYS | 608 | 0.804 | 24.038 | −19.682 | 0.00 | 0.00 | −0.255 | 19.93 | −6.40 |
| ATOM | 4471 | HG | CYS | 608 | 0.788 | 25.271 | −19.157 | 0.00 | 0.00 | 0.190 | 0.00 | 0.00 |
| ATOM | 4472 | N | GLU | 609 | 3.164 | 22.659 | −20.823 | 0.00 | 0.00 | −0.650 | 9.00 | −17.40 |
| ATOM | 4473 | HN | GLU | 609 | 2.520 | 22.352 | −20.080 | 0.00 | 0.00 | 0.439 | 0.00 | 0.00 |
| ATOM | 4474 | CA | GLU | 609 | 4.577 | 22.733 | −20.580 | 0.00 | 0.00 | 0.158 | 9.40 | 4.00 |
| ATOM | 4475 | HA | GLU | 609 | 5.057 | 22.226 | −21.417 | 0.00 | 0.00 | 0.053 | 0.00 | 0.00 |
| ATOM | 4476 | C | GLU | 609 | 4.812 | 22.043 | −19.289 | 0.00 | 0.00 | 0.396 | 9.82 | 4.00 |
| ATOM | 4477 | O | GLU | 609 | 3.898 | 21.385 | −18.805 | 0.00 | 0.00 | −0.396 | 8.17 | −17.40 |
| ATOM | 4478 | CB | GLU | 609 | 5.120 | 24.181 | −20.495 | 0.00 | 0.00 | −0.106 | 12.77 | 4.00 |
| ATOM | 4479 | HB1 | GLU | 609 | 4.951 | 24.662 | −21.458 | 0.00 | 0.00 | 0.053 | 0.00 | 0.00 |
| ATOM | 4480 | HB2 | GLU | 609 | 6.185 | 24.130 | −20.269 | 0.00 | 0.00 | 0.053 | 0.00 | 0.00 |
| ATOM | 4481 | CG | GLU | 609 | 4.468 | 25.064 | −19.423 | 0.00 | 0.00 | −0.336 | 12.77 | 4.00 |
| ATOM | 4482 | HG1 | GLU | 609 | 4.818 | 24.738 | −18.444 | 0.00 | 0.00 | 0.053 | 0.00 | 0.00 |
| ATOM | 4483 | HG2 | GLU | 609 | 3.386 | 24.955 | −19.494 | 0.00 | 0.00 | 0.053 | 0.00 | 0.00 |
| ATOM | 4484 | CD | GLU | 609 | 4.874 | 26.519 | −19.675 | 0.00 | 0.00 | 0.297 | 9.82 | 4.00 |
| ATOM | 4485 | OE1 | GLU | 609 | 4.783 | 26.972 | −20.847 | 0.00 | 0.00 | −0.534 | 8.17 | −18.95 |
| ATOM | 4486 | OE2 | GLU | 609 | 5.278 | 27.201 | −18.696 | 0.00 | 0.00 | −0.534 | 8.17 | −18.95 |
| ATOM | 4487 | N | GLY | 610 | 6.037 | 22.147 | −18.722 | 0.00 | 0.00 | −0.650 | 9.00 | −17.40 |
| ATOM | 4488 | HN | GLY | 610 | 6.767 | 22.699 | −19.195 | 0.00 | 0.00 | 0.439 | 0.00 | 0.00 |
| ATOM | 4489 | CA | GLY | 610 | 6.340 | 21.502 | −17.471 | 0.00 | 0.00 | 0.105 | 9.40 | 4.00 |
| ATOM | 4490 | HA1 | GLY | 610 | 6.380 | 20.426 | −17.637 | 0.00 | 0.00 | 0.053 | 0.00 | 0.00 |
| ATOM | 4491 | HA2 | GLY | 610 | 5.555 | 21.747 | −16.756 | 0.00 | 0.00 | 0.053 | 0.00 | 0.00 |
| ATOM | 4492 | C | GLY | 610 | 7.660 | 22.003 | −16.987 | 0.00 | 0.00 | 0.396 | 9.82 | 4.00 |
| ATOM | 4493 | O | GLY | 610 | 8.653 | 21.953 | −17.704 | 0.00 | 0.00 | −0.396 | 8.17 | −17.40 |
| ATOM | 4494 | N | MET | 611 | 7.640 | 22.533 | −15.746 | 0.00 | 0.00 | −0.650 | 9.00 | −17.40 |
| ATOM | 4495 | HN | MET | 611 | 6.749 | 22.420 | −15.241 | 0.00 | 0.00 | 0.439 | 0.00 | 0.00 |
| ATOM | 4496 | CA | MET | 611 | 8.670 | 23.234 | −15.013 | 0.00 | 0.00 | 0.158 | 9.40 | 4.00 |
| ATOM | 4497 | HA | MET | 611 | 9.197 | 23.842 | −15.749 | 0.00 | 0.00 | 0.053 | 0.00 | 0.00 |
| ATOM | 4498 | C | MET | 611 | 9.790 | 22.546 | −14.226 | 0.00 | 0.00 | 0.396 | 9.82 | 4.00 |
| ATOM | 4499 | O | MET | 611 | 10.947 | 22.920 | −14.401 | 0.00 | 0.00 | −0.396 | 8.17 | −17.40 |
| ATOM | 4500 | CB | MET | 611 | 8.014 | 24.201 | −14.009 | 0.00 | 0.00 | −0.106 | 12.77 | 4.00 |
| ATOM | 4501 | HB1 | MET | 611 | 7.723 | 23.625 | −13.131 | 0.00 | 0.00 | 0.053 | 0.00 | 0.00 |
| ATOM | 4502 | HB2 | MET | 611 | 7.143 | 24.644 | −14.492 | 0.00 | 0.00 | 0.053 | 0.00 | 0.00 |
| ATOM | 4503 | CG | MET | 611 | 8.891 | 25.357 | −13.517 | 0.00 | 0.00 | −0.041 | 12.77 | 4.00 |
| ATOM | 4504 | HG1 | MET | 611 | 9.893 | 24.969 | −13.334 | 0.00 | 0.00 | 0.053 | 0.00 | 0.00 |
| ATOM | 4505 | HG2 | MET | 611 | 8.454 | 25.747 | −12.598 | 0.00 | 0.00 | 0.053 | 0.00 | 0.00 |
| ATOM | 4506 | SD | MET | 611 | 9.048 | 26.747 | −14.680 | 0.00 | 0.00 | −0.130 | 16.39 | −6.40 |
| ATOM | 4507 | CE | MET | 611 | 9.547 | 27.960 | −13.423 | 0.00 | 0.00 | −0.094 | 16.15 | 4.00 |
| ATOM | 4508 | HE1 | MET | 611 | 9.710 | 28.928 | −13.896 | 0.00 | 0.00 | 0.053 | 0.00 | 0.00 |
| ATOM | 4509 | HE2 | MET | 611 | 8.762 | 28.051 | −12.673 | 0.00 | 0.00 | 0.053 | 0.00 | 0.00 |
| ATOM | 4510 | HE3 | MET | 611 | 10.469 | 27.630 | −12.945 | 0.00 | 0.00 | 0.053 | 0.00 | 0.00 |
| ATOM | 4511 | N | LEU | 612 | 9.538 | 21.502 | −13.395 | 0.00 | 0.00 | −0.650 | 9.00 | −17.40 |
| ATOM | 4512 | HN | LEU | 612 | 8.722 | 20.894 | −13.555 | 0.00 | 0.00 | 0.439 | 0.00 | 0.00 |
| ATOM | 4513 | CA | LEU | 612 | 10.436 | 21.251 | −12.274 | 0.00 | 0.00 | 0.158 | 9.40 | 4.00 |
| ATOM | 4514 | HA | LEU | 612 | 10.249 | 22.038 | −11.543 | 0.00 | 0.00 | 0.053 | 0.00 | 0.00 |
| ATOM | 4515 | C | LEU | 612 | 11.923 | 21.233 | −12.463 | 0.00 | 0.00 | 0.396 | 9.82 | 4.00 |
| ATOM | 4516 | O | LEU | 612 | 12.553 | 22.205 | −12.060 | 0.00 | 0.00 | −0.396 | 8.17 | −17.40 |
| ATOM | 4517 | CB | LEU | 612 | 10.101 | 20.012 | −11.422 | 0.00 | 0.00 | −0.106 | 12.77 | 4.00 |
| ATOM | 4518 | HB1 | LEU | 612 | 10.967 | 19.613 | −10.894 | 0.00 | 0.00 | 0.053 | 0.00 | 0.00 |
| ATOM | 4519 | HB2 | LEU | 612 | 9.706 | 19.185 | −12.012 | 0.00 | 0.00 | 0.053 | 0.00 | 0.00 |
| ATOM | 4520 | CG | LEU | 612 | 9.047 | 20.295 | −10.342 | 0.00 | 0.00 | −0.053 | 9.40 | 4.00 |
| ATOM | 4521 | HG | LEU | 612 | 8.687 | 19.369 | −9.894 | 0.00 | 0.00 | 0.053 | 0.00 | 0.00 |
| ATOM | 4522 | CD1 | LEU | 612 | 9.629 | 21.167 | −9.217 | 0.00 | 0.00 | −0.159 | 16.15 | 4.00 |
| ATOM | 4523 | HD11 | LEU | 612 | 8.861 | 21.352 | −8.466 | 0.00 | 0.00 | 0.053 | 0.00 | 0.00 |
| ATOM | 4524 | HD12 | LEU | 612 | 10.471 | 20.651 | −8.756 | 0.00 | 0.00 | 0.053 | 0.00 | 0.00 |
| ATOM | 4525 | HD13 | LEU | 612 | 9.968 | 22.117 | −9.631 | 0.00 | 0.00 | 0.053 | 0.00 | 0.00 |
| ATOM | 4526 | CD2 | LEU | 612 | 7.804 | 20.934 | −10.956 | 0.00 | 0.00 | −0.159 | 16.15 | 4.00 |
| ATOM | 4527 | HD21 | LEU | 612 | 7.070 | 21.126 | −10.174 | 0.00 | 0.00 | 0.053 | 0.00 | 0.00 |
| ATOM | 4528 | HD22 | LEU | 612 | 8.077 | 21.874 | −11.436 | 0.00 | 0.00 | 0.053 | 0.00 | 0.00 |
| ATOM | 4529 | HD23 | LEU | 612 | 7.376 | 20.259 | −11.697 | 0.00 | 0.00 | 0.053 | 0.00 | 0.00 |
| ATOM | 4530 | N | GLN | 613 | 12.543 | 20.229 | −13.110 | 0.00 | 0.00 | −0.650 | 9.00 | −17.40 |
| ATOM | 4531 | HN | GLN | 613 | 12.008 | 19.532 | −13.647 | 0.00 | 0.00 | 0.439 | 0.00 | 0.00 |
| ATOM | 4532 | CA | GLN | 613 | 13.989 | 20.161 | −13.026 | 0.00 | 0.00 | 0.158 | 9.40 | 4.00 |
| ATOM | 4533 | HA | GLN | 613 | 14.369 | 19.293 | −13.565 | 0.00 | 0.00 | 0.053 | 0.00 | 0.00 |
| ATOM | 4534 | C | GLN | 613 | 14.439 | 20.054 | −11.593 | 0.00 | 0.00 | 0.396 | 9.82 | 4.00 |
| ATOM | 4535 | O | GLN | 613 | 14.311 | 20.963 | −10.773 | 0.00 | 0.00 | −0.396 | 8.17 | −17.40 |
| ATOM | 4536 | CB | GLN | 613 | 14.819 | 21.322 | −13.609 | 0.00 | 0.00 | −0.106 | 12.77 | 4.00 |
| ATOM | 4537 | HB1 | GLN | 613 | 15.767 | 21.355 | −13.073 | 0.00 | 0.00 | 0.053 | 0.00 | 0.00 |
| ATOM | 4538 | HB2 | GLN | 613 | 14.253 | 22.242 | −13.463 | 0.00 | 0.00 | 0.053 | 0.00 | 0.00 |
| ATOM | 4539 | CG | GLN | 613 | 15.153 | 21.238 | −15.097 | 0.00 | 0.00 | −0.106 | 12.77 | 4.00 |
| ATOM | 4540 | HG1 | GLN | 613 | 14.328 | 21.748 | −15.593 | 0.00 | 0.00 | 0.053 | 0.00 | 0.00 |
| ATOM | 4541 | HG2 | GLN | 613 | 15.202 | 20.170 | −15.308 | 0.00 | 0.00 | 0.053 | 0.00 | 0.00 |
| ATOM | 4542 | CD | GLN | 613 | 16.494 | 21.956 | −15.232 | 0.00 | 0.00 | 0.396 | 9.82 | 4.00 |
| ATOM | 4543 | OE1 | GLN | 613 | 16.644 | 23.036 | −15.800 | 0.00 | 0.00 | −0.396 | 8.17 | −17.40 |
| ATOM | 4544 | NE2 | GLN | 613 | 17.537 | 21.315 | −14.641 | 0.00 | 0.00 | −0.879 | 13.25 | −17.40 |
| ATOM | 4545 | HE21 | GLN | 613 | 17.386 | 20.411 | −14.171 | 0.00 | 0.00 | 0.439 | 0.00 | 0.00 |
| ATOM | 4546 | HE22 | GLN | 613 | 18.478 | 21.733 | −14.663 | 0.00 | 0.00 | 0.439 | 0.00 | 0.00 |
| ATOM | 4547 | N | ARG | 614 | 15.014 | 18.893 | −11.262 | 0.00 | 0.00 | −0.650 | 9.00 | −17.40 |
| ATOM | 4548 | HN | ARG | 614 | 15.144 | 18.190 | −12.004 | 0.00 | 0.00 | 0.439 | 0.00 | 0.00 |
| ATOM | 4549 | CA | ARG | 614 | 15.461 | 18.564 | −9.940 | 0.00 | 0.00 | 0.158 | 9.40 | 4.00 |
| ATOM | 4550 | HA | ARG | 614 | 14.617 | 18.700 | −9.263 | 0.00 | 0.00 | 0.053 | 0.00 | 0.00 |
| ATOM | 4551 | C | ARG | 614 | 16.605 | 19.381 | −9.378 | 0.00 | 0.00 | 0.396 | 9.82 | 4.00 |
| ATOM | 4552 | O | ARG | 614 | 16.430 | 20.088 | −8.384 | 0.00 | 0.00 | −0.396 | 8.17 | −17.40 |
| ATOM | 4553 | CB | ARG | 614 | 15.802 | 17.058 | −9.902 | 0.00 | 0.00 | −0.106 | 12.77 | 4.00 |
| ATOM | 4554 | HB1 | ARG | 614 | 14.867 | 16.498 | −9.899 | 0.00 | 0.00 | 0.053 | 0.00 | 0.00 |
| ATOM | 4555 | HB2 | ARG | 614 | 16.373 | 16.862 | −8.994 | 0.00 | 0.00 | 0.053 | 0.00 | 0.00 |
| ATOM | 4556 | CG | ARG | 614 | 16.641 | 16.549 | −11.095 | 0.00 | 0.00 | −0.106 | 12.77 | 4.00 |
| ATOM | 4557 | HG1 | ARG | 614 | 16.279 | 17.056 | −11.989 | 0.00 | 0.00 | 0.053 | 0.00 | 0.00 |
| ATOM | 4558 | HG2 | ARG | 614 | 16.494 | 15.471 | −11.161 | 0.00 | 0.00 | 0.053 | 0.00 | 0.00 |
| ATOM | 4559 | CD | ARG | 614 | 18.149 | 16.778 | −11.050 | 0.00 | 0.00 | 0.374 | 12.77 | 4.00 |
| ATOM | 4560 | HD1 | ARG | 614 | 18.550 | 16.217 | −10.205 | 0.00 | 0.00 | 0.053 | 0.00 | 0.00 |
| ATOM | 4561 | HD2 | ARG | 614 | 18.324 | 17.847 | −10.925 | 0.00 | 0.00 | 0.053 | 0.00 | 0.00 |
| ATOM | 4562 | NE | ARG | 614 | 18.728 | 16.290 | −12.336 | 0.00 | 0.00 | −0.819 | 9.00 | −24.67 |
| ATOM | 4563 | HE | ARG | 614 | 18.612 | 16.844 | −13.196 | 0.00 | 0.00 | 0.407 | 0.00 | 0.00 |
| ATOM | 4564 | CZ | ARG | 614 | 19.411 | 15.109 | −12.371 | 0.00 | 0.00 | 0.796 | 6.95 | 4.00 |
| ATOM | 4565 | NH1 | ARG | 614 | 19.667 | 14.437 | −11.212 | 0.00 | 0.00 | −0.746 | 9.00 | −24.67 |
| ATOM | 4566 | HH11 | ARG | 614 | 20.182 | 13.546 | −11.237 | 0.00 | 0.00 | 0.407 | 0.00 | 0.00 |
| ATOM | 4567 | HH12 | ARG | 614 | 19.345 | 14.821 | −10.312 | 0.00 | 0.00 | 0.407 | 0.00 | 0.00 |
| ATOM | 4568 | NH2 | ARG | 614 | 19.856 | 14.606 | −13.558 | 0.00 | 0.00 | −0.746 | 9.00 | −24.67 |
| ATOM | 4569 | HH21 | ARG | 614 | 20.371 | 13.714 | −13.579 | 0.00 | 0.00 | 0.407 | 0.00 | 0.00 |
| ATOM | 4570 | HH22 | ARG | 614 | 19.677 | 15.118 | −14.434 | 0.00 | 0.00 | 0.407 | 0.00 | 0.00 |
| ATOM | 4571 | N | ILE | 615 | 17.782 | 19.368 | −10.037 | 0.00 | 0.00 | −0.650 | 9.00 | −17.40 |
| ATOM | 4572 | HN | ILE | 615 | 17.810 | 19.004 | −11.000 | 0.00 | 0.00 | 0.439 | 0.00 | 0.00 |
| ATOM | 4573 | CA | ILE | 615 | 19.003 | 19.847 | −9.436 | 0.00 | 0.00 | 0.158 | 9.40 | 4.00 |
| ATOM | 4574 | HA | ILE | 615 | 19.170 | 19.283 | −8.518 | 0.00 | 0.00 | 0.053 | 0.00 | 0.00 |
| ATOM | 4575 | C | ILE | 615 | 19.019 | 21.294 | −9.074 | 0.00 | 0.00 | 0.396 | 9.82 | 4.00 |
| ATOM | 4576 | O | ILE | 615 | 19.280 | 21.630 | −7.920 | 0.00 | 0.00 | −0.396 | 8.17 | −17.40 |
| ATOM | 4577 | CB | ILE | 615 | 20.222 | 19.587 | −10.290 | 0.00 | 0.00 | −0.053 | 9.40 | 4.00 |
| ATOM | 4578 | HB | ILE | 615 | 19.960 | 19.749 | −11.336 | 0.00 | 0.00 | 0.053 | 0.00 | 0.00 |
| ATOM | 4579 | CG1 | ILE | 615 | 20.636 | 18.104 | −10.244 | 0.00 | 0.00 | −0.106 | 12.77 | 4.00 |
| ATOM | 4580 | HG11 | ILE | 615 | 21.539 | 17.984 | −10.843 | 0.00 | 0.00 | 0.053 | 0.00 | 0.00 |
| ATOM | 4581 | HG12 | ILE | 615 | 19.819 | 17.510 | −10.654 | 0.00 | 0.00 | 0.053 | 0.00 | 0.00 |
| ATOM | 4582 | CG2 | ILE | 615 | 21.337 | 20.554 | −9.869 | 0.00 | 0.00 | −0.159 | 16.15 | 4.00 |
| ATOM | 4583 | HG21 | ILE | 615 | 22.223 | 20.376 | −10.479 | 0.00 | 0.00 | 0.053 | 0.00 | 0.00 |
| ATOM | 4584 | HG22 | ILE | 615 | 21.000 | 21.581 | −10.010 | 0.00 | 0.00 | 0.053 | 0.00 | 0.00 |
| ATOM | 4585 | HG23 | ILE | 615 | 21.581 | 20.393 | −8.819 | 0.00 | 0.00 | 0.053 | 0.00 | 0.00 |
| ATOM | 4586 | CD1 | ILE | 615 | 20.938 | 17.565 | −8.844 | 0.00 | 0.00 | −0.159 | 16.15 | 4.00 |
| ATOM | 4587 | HD11 | ILE | 615 | 21.220 | 16.514 | −8.912 | 0.00 | 0.00 | 0.053 | 0.00 | 0.00 |
| ATOM | 4588 | HD12 | ILE | 615 | 21.758 | 18.133 | −8.404 | 0.00 | 0.00 | 0.053 | 0.00 | 0.00 |
| ATOM | 4589 | HD13 | ILE | 615 | 20.051 | 17.663 | −8.217 | 0.00 | 0.00 | 0.053 | 0.00 | 0.00 |
| ATOM | 4590 | N | ASN | 616 | 18.740 | 22.201 | −10.020 | 0.00 | 0.00 | −0.650 | 9.00 | −17.40 |
| ATOM | 4591 | HN | ASN | 616 | 18.401 | 21.934 | −10.955 | 0.00 | 0.00 | 0.439 | 0.00 | 0.00 |
| ATOM | 4592 | CA | ASN | 616 | 18.945 | 23.566 | −9.642 | 0.00 | 0.00 | 0.158 | 9.40 | 4.00 |
| ATOM | 4593 | HA | ASN | 616 | 19.859 | 23.717 | −9.067 | 0.00 | 0.00 | 0.053 | 0.00 | 0.00 |
| ATOM | 4594 | C | ASN | 616 | 17.837 | 24.113 | −8.792 | 0.00 | 0.00 | 0.396 | 9.82 | 4.00 |
| ATOM | 4595 | O | ASN | 616 | 18.071 | 24.552 | −7.665 | 0.00 | 0.00 | −0.396 | 8.17 | −17.40 |
| ATOM | 4596 | CB | ASN | 616 | 19.149 | 24.500 | −10.844 | 0.00 | 0.00 | −0.106 | 12.77 | 4.00 |
| ATOM | 4597 | HB1 | ASN | 616 | 18.198 | 24.556 | −11.374 | 0.00 | 0.00 | 0.053 | 0.00 | 0.00 |
| ATOM | 4598 | HB2 | ASN | 616 | 19.930 | 24.061 | −11.465 | 0.00 | 0.00 | 0.053 | 0.00 | 0.00 |
| ATOM | 4599 | CG | ASN | 616 | 19.566 | 25.858 | −10.294 | 0.00 | 0.00 | 0.396 | 9.82 | 4.00 |
| ATOM | 4600 | OD1 | ASN | 616 | 20.506 | 25.974 | −9.508 | 0.00 | 0.00 | −0.396 | 8.17 | −17.40 |
| ATOM | 4601 | ND2 | ASN | 616 | 18.833 | 26.923 | −10.715 | 0.00 | 0.00 | −0.879 | 13.25 | −17.40 |
| ATOM | 4602 | HD21 | ASN | 616 | 18.054 | 26.784 | −11.374 | 0.00 | 0.00 | 0.439 | 0.00 | 0.00 |
| ATOM | 4603 | HD22 | ASN | 616 | 19.057 | 27.869 | −10.375 | 0.00 | 0.00 | 0.439 | 0.00 | 0.00 |
| ATOM | 4604 | N | ALA | 617 | 16.594 | 24.096 | −9.302 | 0.00 | 0.00 | −0.650 | 9.00 | −17.40 |
| ATOM | 4605 | HN | ALA | 617 | 16.415 | 23.615 | −10.195 | 0.00 | 0.00 | 0.439 | 0.00 | 0.00 |
| ATOM | 4606 | CA | ALA | 617 | 15.512 | 24.744 | −8.612 | 0.00 | 0.00 | 0.158 | 9.40 | 4.00 |
| ATOM | 4607 | HA | ALA | 617 | 15.742 | 25.774 | −8.340 | 0.00 | 0.00 | 0.053 | 0.00 | 0.00 |
| ATOM | 4608 | C | ALA | 617 | 15.141 | 24.052 | −7.333 | 0.00 | 0.00 | 0.351 | 9.82 | 4.00 |
| ATOM | 4609 | HC | ALA | 617 | 15.068 | 22.965 | −7.308 | 0.00 | 0.00 | 0.046 | 0.00 | 0.00 |
| ATOM | 4610 | O | ALA | 617 | 14.919 | 24.702 | −6.312 | 0.00 | 0.00 | −0.396 | 8.17 | −17.40 |
| ATOM | 4611 | CB | ALA | 617 | 14.245 | 24.857 | −9.478 | 0.00 | 0.00 | −0.159 | 16.15 | 4.00 |
| ATOM | 4612 | HB1 | ALA | 617 | 13.459 | 25.354 | −8.909 | 0.00 | 0.00 | 0.053 | 0.00 | 0.00 |
| ATOM | 4613 | HB2 | ALA | 617 | 14.467 | 25.437 | −10.374 | 0.00 | 0.00 | 0.053 | 0.00 | 0.00 |
| ATOM | 4614 | HB3 | ALA | 617 | 13.910 | 23.860 | −9.765 | 0.00 | 0.00 | 0.053 | 0.00 | 0.00 |
| HETATM | 4615 | MN | HET | 618 | −0.563 | −1.991 | −4.585 | 1.00 | 0.00 | 0.700 | 9.00 | −17.40 |
| END | | | | | | | | | | | | |
| TER | | | | | | | | | | | | |

TABLE 3
Core 2L Human Model

REMARK

| | | | | | | | | | | | | |
|---|---|---|---|---|---|---|---|---|---|---|---|---|
| ATOM | 1 | N | LEU | 1 | −25.979 | 2.396 | 25.240 | 1.00 | 0.00 | −0.580 | 9.00 | −17.40 |
| ATOM | 2 | HN1 | LEU | 1 | −26.359 | 2.628 | 26.168 | 1.00 | 0.00 | 0.249 | 0.00 | 0.00 |
| ATOM | 3 | HN2 | LEU | 1 | −26.138 | 1.397 | 25.044 | 1.00 | 0.00 | 0.249 | 0.00 | 0.00 |
| ATOM | 4 | CA | LEU | 1 | −26.657 | 3.216 | 24.209 | 1.00 | 0.00 | 0.030 | 9.40 | 4.00 |
| ATOM | 5 | HA | LEU | 1 | −27.638 | 2.793 | 23.991 | 1.00 | 0.00 | 0.053 | 0.00 | 0.00 |
| ATOM | 6 | C | LEU | 1 | −25.839 | 3.230 | 22.962 | 1.00 | 0.00 | 0.396 | 9.82 | 4.00 |
| ATOM | 7 | O | LEU | 1 | −25.371 | 2.190 | 22.502 | 1.00 | 0.00 | −0.396 | 8.17 | −17.40 |
| ATOM | 8 | CB | LEU | 1 | −26.823 | 4.665 | 24.696 | 1.00 | 0.00 | −0.106 | 12.77 | 4.00 |
| ATOM | 9 | HB1 | LEU | 1 | −27.281 | 5.326 | 23.960 | 1.00 | 0.00 | 0.053 | 0.00 | 0.00 |
| ATOM | 10 | HB2 | LEU | 1 | −25.883 | 5.150 | 24.960 | 1.00 | 0.00 | 0.053 | 0.00 | 0.00 |
| ATOM | 11 | CG | LEU | 1 | −27.705 | 4.801 | 25.953 | 1.00 | 0.00 | −0.053 | 9.40 | 4.00 |
| ATOM | 12 | HG | LEU | 1 | −27.275 | 4.264 | 26.798 | 1.00 | 0.00 | 0.053 | 0.00 | 0.00 |
| ATOM | 13 | CD1 | LEU | 1 | −27.862 | 6.272 | 26.370 | 1.00 | 0.00 | −0.159 | 16.15 | 4.00 |
| ATOM | 14 | HD1 | LEU | 1 | −28.489 | 6.333 | 27.259 | 1.00 | 0.00 | 0.053 | 0.00 | 0.00 |
| ATOM | 15 | HD1 | LEU | 1 | −26.881 | 6.695 | 26.588 | 1.00 | 0.00 | 0.053 | 0.00 | 0.00 |
| ATOM | 16 | HD1 | LEU | 1 | −28.326 | 6.832 | 25.558 | 1.00 | 0.00 | 0.053 | 0.00 | 0.00 |
| ATOM | 17 | CD2 | LEU | 1 | −29.053 | 4.084 | 25.779 | 1.00 | 0.00 | −0.159 | 16.15 | 4.00 |
| ATOM | 18 | HD2 | LEU | 1 | −29.647 | 4.201 | 26.685 | 1.00 | 0.00 | 0.053 | 0.00 | 0.00 |
| ATOM | 19 | HD2 | LEU | 1 | −29.589 | 4.517 | 24.934 | 1.00 | 0.00 | 0.053 | 0.00 | 0.00 |
| ATOM | 20 | HD2 | LEU | 1 | −28.880 | 3.023 | 25.593 | 1.00 | 0.00 | 0.053 | 0.00 | 0.00 |
| ATOM | 21 | N | ALA | 2 | −25.654 | 4.424 | 22.370 | 1.00 | 0.00 | −0.650 | 9.00 | −17.40 |
| ATOM | 22 | HN | ALA | 2 | −26.079 | 5.272 | 22.770 | 1.00 | 0.00 | 0.440 | 0.00 | 0.00 |
| ATOM | 23 | CA | ALA | 2 | −24.862 | 4.507 | 21.180 | 1.00 | 0.00 | 0.158 | 9.40 | 4.00 |
| ATOM | 24 | HA | ALA | 2 | −25.128 | 3.643 | 20.570 | 1.00 | 0.00 | 0.053 | 0.00 | 0.00 |
| ATOM | 25 | C | ALA | 2 | −23.437 | 4.472 | 21.617 | 1.00 | 0.00 | 0.396 | 9.82 | 4.00 |
| ATOM | 26 | O | ALA | 2 | −23.143 | 4.208 | 22.782 | 1.00 | 0.00 | −0.396 | 8.17 | −17.40 |
| ATOM | 27 | CB | ALA | 2 | −25.077 | 5.805 | 20.385 | 1.00 | 0.00 | −0.159 | 16.15 | 4.00 |
| ATOM | 28 | HB1 | ALA | 2 | −24.444 | 5.797 | 19.497 | 1.00 | 0.00 | 0.053 | 0.00 | 0.00 |
| ATOM | 29 | HB2 | ALA | 2 | −26.122 | 5.879 | 20.084 | 1.00 | 0.00 | 0.053 | 0.00 | 0.00 |
| ATOM | 30 | HB3 | ALA | 2 | −24.817 | 6.660 | 21.008 | 1.00 | 0.00 | 0.053 | 0.00 | 0.00 |
| ATOM | 31 | N | GLY | 3 | −22.500 | 4.725 | 20.687 | 1.00 | 0.00 | −0.650 | 9.00 | −17.40 |
| ATOM | 32 | HN | GLY | 3 | −22.763 | 4.924 | 19.711 | 1.00 | 0.00 | 0.440 | 0.00 | 0.00 |
| ATOM | 33 | CA | GLY | 3 | −21.128 | 4.710 | 21.087 | 1.00 | 0.00 | 0.105 | 9.40 | 4.00 |
| ATOM | 34 | HA1 | GLY | 3 | −21.000 | 3.907 | 21.813 | 1.00 | 0.00 | 0.053 | 0.00 | 0.00 |
| ATOM | 35 | HA2 | GLY | 3 | −20.898 | 5.678 | 21.530 | 1.00 | 0.00 | 0.053 | 0.00 | 0.00 |
| ATOM | 36 | C | GLY | 3 | −20.301 | 4.469 | 19.873 | 1.00 | 0.00 | 0.396 | 9.82 | 4.00 |
| ATOM | 37 | O | GLY | 3 | −19.427 | 3.604 | 19.864 | 1.00 | 0.00 | −0.396 | 8.17 | −17.40 |
| ATOM | 38 | N | GLU | 4 | −20.564 | 5.240 | 18.804 | 1.00 | 0.00 | −0.650 | 9.00 | −17.40 |
| ATOM | 39 | HN | GLU | 4 | −21.320 | 5.938 | 18.844 | 1.00 | 0.00 | 0.440 | 0.00 | 0.00 |
| ATOM | 40 | CA | GLU | 4 | −19.793 | 5.089 | 17.612 | 1.00 | 0.00 | 0.158 | 9.40 | 4.00 |
| ATOM | 41 | HA | GLU | 4 | −19.336 | 4.102 | 17.686 | 1.00 | 0.00 | 0.053 | 0.00 | 0.00 |
| ATOM | 42 | C | GLU | 4 | −18.797 | 6.204 | 17.640 | 1.00 | 0.00 | 0.396 | 9.82 | 4.00 |
| ATOM | 43 | O | GLU | 4 | −19.154 | 7.373 | 17.496 | 1.00 | 0.00 | −0.396 | 8.17 | −17.40 |
| ATOM | 44 | CB | GLU | 4 | −20.660 | 5.211 | 16.342 | 1.00 | 0.00 | −0.106 | 12.77 | 4.00 |
| ATOM | 45 | HB1 | GLU | 4 | −20.824 | 6.270 | 16.144 | 1.00 | 0.00 | 0.053 | 0.00 | 0.00 |
| ATOM | 46 | HB2 | GLU | 4 | −21.605 | 4.700 | 16.526 | 1.00 | 0.00 | 0.053 | 0.00 | 0.00 |
| ATOM | 47 | CG | GLU | 4 | −20.048 | 4.599 | 15.080 | 1.00 | 0.00 | −0.106 | 12.77 | 4.00 |
| ATOM | 48 | HG1 | GLU | 4 | −20.851 | 4.299 | 14.407 | 1.00 | 0.00 | 0.053 | 0.00 | 0.00 |
| ATOM | 49 | HG2 | GLU | 4 | −19.453 | 3.729 | 15.361 | 1.00 | 0.00 | 0.053 | 0.00 | 0.00 |
| ATOM | 50 | CD | GLU | 4 | −19.166 | 5.637 | 14.408 | 1.00 | 0.00 | 0.399 | 9.82 | 4.00 |
| ATOM | 51 | OE1 | GLU | 4 | −19.066 | 6.771 | 14.945 | 1.00 | 0.00 | −0.396 | 8.17 | −18.95 |
| ATOM | 52 | OE2 | GLU | 4 | −18.582 | 5.308 | 13.341 | 1.00 | 0.00 | −0.427 | 8.17 | −18.95 |
| ATOM | 54 | N | ASN | 5 | −17.511 | 5.867 | 17.864 | 1.00 | 0.00 | −0.650 | 9.00 | −17.40 |
| ATOM | 55 | HN | ASN | 5 | −17.250 | 4.879 | 17.993 | 1.00 | 0.00 | 0.440 | 0.00 | 0.00 |
| ATOM | 56 | CA | ASN | 5 | −16.507 | 6.890 | 17.921 | 1.00 | 0.00 | 0.158 | 9.40 | 4.00 |
| ATOM | 57 | HA | ASN | 5 | −17.009 | 7.854 | 17.998 | 1.00 | 0.00 | 0.053 | 0.00 | 0.00 |
| ATOM | 58 | C | ASN | 5 | −15.695 | 6.814 | 16.670 | 1.00 | 0.00 | 0.396 | 9.82 | 4.00 |
| ATOM | 59 | O | ASN | 5 | −15.019 | 5.826 | 16.385 | 1.00 | 0.00 | −0.396 | 8.17 | −17.40 |
| ATOM | 60 | CB | ASN | 5 | −15.567 | 6.767 | 19.131 | 1.00 | 0.00 | −0.106 | 12.77 | 4.00 |
| ATOM | 61 | HB1 | ASN | 5 | −16.160 | 6.879 | 20.038 | 1.00 | 0.00 | 0.053 | 0.00 | 0.00 |
| ATOM | 62 | HB2 | ASN | 5 | −14.816 | 7.554 | 19.063 | 1.00 | 0.00 | 0.053 | 0.00 | 0.00 |
| ATOM | 63 | CG | ASN | 5 | −14.910 | 5.398 | 19.088 | 1.00 | 0.00 | 0.396 | 9.82 | 4.00 |
| ATOM | 64 | OD1 | ASN | 5 | −13.819 | 5.223 | 18.547 | 1.00 | 0.00 | −0.396 | 8.17 | −17.40 |
| ATOM | 65 | ND2 | ASN | 5 | −15.599 | 4.392 | 19.692 | 1.00 | 0.00 | −0.879 | 13.25 | −17.40 |
| ATOM | 66 | HD2 | ASN | 5 | −16.509 | 4.584 | 20.132 | 1.00 | 0.00 | 0.440 | 0.00 | 0.00 |
| ATOM | 67 | HD2 | ASN | 5 | −15.209 | 3.438 | 19.708 | 1.00 | 0.00 | 0.440 | 0.00 | 0.00 |
| ATOM | 68 | N | PRO | 6 | −15.800 | 7.865 | 15.904 | 1.00 | 0.00 | −0.422 | 9.00 | −17.40 |
| ATOM | 69 | CA | PRO | 6 | −15.083 | 7.940 | 14.663 | 1.00 | 0.00 | 0.158 | 9.40 | 4.00 |
| ATOM | 70 | HA | PRO | 6 | −15.042 | 6.964 | 14.178 | 1.00 | 0.00 | 0.053 | 0.00 | 0.00 |
| ATOM | 71 | CD | PRO | 6 | −17.036 | 8.628 | 15.863 | 1.00 | 0.00 | 0.105 | 12.77 | 4.00 |
| ATOM | 72 | HD1 | PRO | 6 | −17.098 | 9.100 | 16.843 | 1.00 | 0.00 | 0.053 | 0.00 | 0.00 |
| ATOM | 73 | HD2 | PRO | 6 | −17.811 | 7.883 | 15.681 | 1.00 | 0.00 | 0.053 | 0.00 | 0.00 |
| ATOM | 74 | C | PRO | 6 | −13.685 | 8.403 | 14.886 | 1.00 | 0.00 | 0.396 | 9.82 | 4.00 |
| ATOM | 75 | O | PRO | 6 | −13.437 | 9.134 | 15.843 | 1.00 | 0.00 | −0.396 | 8.17 | −17.40 |
| ATOM | 76 | CB | PRO | 6 | −15.892 | 8.870 | 13.753 | 1.00 | 0.00 | −0.106 | 12.77 | 4.00 |
| ATOM | 77 | HB1 | PRO | 6 | −16.401 | 8.201 | 13.059 | 1.00 | 0.00 | 0.053 | 0.00 | 0.00 |
| ATOM | 78 | HB2 | PRO | 6 | −15.146 | 9.510 | 13.280 | 1.00 | 0.00 | 0.053 | 0.00 | 0.00 |
| ATOM | 79 | CG | PRO | 6 | −16.840 | 9.618 | 14.706 | 1.00 | 0.00 | −0.106 | 12.77 | 4.00 |
| ATOM | 80 | HG1 | PRO | 6 | −17.782 | 9.855 | 14.213 | 1.00 | 0.00 | 0.053 | 0.00 | 0.00 |
| ATOM | 81 | HG2 | PRO | 6 | −16.395 | 10.553 | 15.045 | 1.00 | 0.00 | 0.053 | 0.00 | 0.00 |
| ATOM | 82 | N | SER | 7 | −12.751 | 7.982 | 14.016 | 1.00 | 0.00 | −0.650 | 9.00 | −17.40 |
| ATOM | 83 | HN | SER | 7 | −12.993 | 7.317 | 13.267 | 1.00 | 0.00 | 0.440 | 0.00 | 0.00 |
| ATOM | 84 | CA | SER | 7 | −11.415 | 8.472 | 14.148 | 1.00 | 0.00 | 0.158 | 9.40 | 4.00 |
| ATOM | 85 | HA | SER | 7 | −11.354 | 8.927 | 15.136 | 1.00 | 0.00 | 0.053 | 0.00 | 0.00 |
| ATOM | 86 | C | SER | 7 | −11.226 | 9.458 | 13.055 | 1.00 | 0.00 | 0.396 | 9.82 | 4.00 |
| ATOM | 87 | O | SER | 7 | −11.467 | 9.155 | 11.888 | 1.00 | 0.00 | −0.396 | 8.17 | −17.40 |
| ATOM | 88 | CB | SER | 7 | −10.319 | 7.411 | 13.969 | 1.00 | 0.00 | 0.007 | 12.77 | 4.00 |
| ATOM | 89 | HB1 | SER | 7 | −10.384 | 6.969 | 12.974 | 1.00 | 0.00 | 0.053 | 0.00 | 0.00 |
| ATOM | 90 | HB2 | SER | 7 | −10.435 | 6.622 | 14.712 | 1.00 | 0.00 | 0.053 | 0.00 | 0.00 |
| ATOM | 91 | OG | SER | 7 | −9.036 | 8.003 | 14.125 | 1.00 | 0.00 | −0.537 | 11.04 | −17.40 |
| ATOM | 92 | HG | SER | 7 | −8.321 | 7.389 | 13.707 | 1.00 | 0.00 | 0.424 | 0.00 | 0.00 |
| ATOM | 93 | N | SER | 8 | −10.804 | 10.685 | 13.409 | 1.00 | 0.00 | −0.650 | 9.00 | −17.40 |
| ATOM | 94 | HN | SER | 8 | −10.641 | 10.929 | 14.396 | 1.00 | 0.00 | 0.440 | 0.00 | 0.00 |
| ATOM | 95 | CA | SER | 8 | −10.589 | 11.636 | 12.366 | 1.00 | 0.00 | 0.158 | 9.40 | 4.00 |
| ATOM | 96 | HA | SER | 8 | −11.084 | 11.271 | 11.466 | 1.00 | 0.00 | 0.053 | 0.00 | 0.00 |
| ATOM | 97 | C | SER | 8 | −9.123 | 11.760 | 12.146 | 1.00 | 0.00 | 0.396 | 9.82 | 4.00 |
| ATOM | 98 | O | SER | 8 | −8.351 | 12.030 | 13.066 | 1.00 | 0.00 | −0.396 | 8.17 | −17.40 |
| ATOM | 99 | CB | SER | 8 | −11.078 | 13.052 | 12.647 | 1.00 | 0.00 | 0.007 | 12.77 | 4.00 |
| ATOM | 100 | HB1 | SER | 8 | −10.624 | 13.405 | 13.572 | 1.00 | 0.00 | 0.053 | 0.00 | 0.00 |
| ATOM | 101 | HB2 | SER | 8 | −12.163 | 13.038 | 12.745 | 1.00 | 0.00 | 0.053 | 0.00 | 0.00 |
| ATOM | 102 | OG | SER | 8 | −10.699 | 13.891 | 11.570 | 1.00 | 0.00 | −0.537 | 11.04 | −17.40 |
| ATOM | 103 | HG | SER | 8 | −10.032 | 14.600 | 11.905 | 1.00 | 0.00 | 0.424 | 0.00 | 0.00 |
| ATOM | 104 | N | ASP | 9 | −8.702 | 11.568 | 10.888 | 1.00 | 0.00 | −0.650 | 9.00 | −17.40 |
| ATOM | 105 | HN | ASP | 9 | −9.377 | 11.342 | 10.143 | 1.00 | 0.00 | 0.440 | 0.00 | 0.00 |
| ATOM | 106 | CA | ASP | 9 | −7.310 | 11.679 | 10.587 | 1.00 | 0.00 | 0.158 | 9.40 | 4.00 |
| ATOM | 107 | HA | ASP | 9 | −6.781 | 11.731 | 11.538 | 1.00 | 0.00 | 0.053 | 0.00 | 0.00 |
| ATOM | 108 | C | ASP | 9 | −7.135 | 12.926 | 9.785 | 1.00 | 0.00 | 0.396 | 9.82 | 4.00 |
| ATOM | 109 | O | ASP | 9 | −7.899 | 13.188 | 8.858 | 1.00 | 0.00 | −0.396 | 8.17 | −17.40 |
| ATOM | 110 | CB | ASP | 9 | −6.774 | 10.512 | 9.735 | 1.00 | 0.00 | −0.336 | 12.77 | 4.00 |
| ATOM | 111 | HB1 | ASP | 9 | −7.171 | 10.621 | 8.726 | 1.00 | 0.00 | 0.053 | 0.00 | 0.00 |
| ATOM | 112 | HB2 | ASP | 9 | −7.113 | 9.580 | 10.188 | 1.00 | 0.00 | 0.053 | 0.00 | 0.00 |
| ATOM | 113 | CG | ASP | 9 | −5.254 | 10.592 | 9.732 | 1.00 | 0.00 | 0.297 | 9.82 | 4.00 |
| ATOM | 114 | OD1 | ASP | 9 | −4.690 | 11.150 | 10.710 | 1.00 | 0.00 | −0.534 | 8.17 | −18.95 |
| ATOM | 115 | OD2 | ASP | 9 | −4.634 | 10.102 | 8.751 | 1.00 | 0.00 | −0.534 | 8.17 | −18.95 |
| ATOM | 116 | N | ILE | 10 | −6.124 | 13.745 | 10.132 | 1.00 | 0.00 | −0.650 | 9.00 | −17.40 |
| ATOM | 117 | HN | ILE | 10 | −5.513 | 13.513 | 10.928 | 1.00 | 0.00 | 0.440 | 0.00 | 0.00 |
| ATOM | 118 | CA | ILE | 10 | −5.910 | 14.944 | 9.377 | 1.00 | 0.00 | 0.158 | 9.40 | 4.00 |
| ATOM | 119 | HA | ILE | 10 | −6.821 | 15.142 | 8.812 | 1.00 | 0.00 | 0.053 | 0.00 | 0.00 |
| ATOM | 120 | C | ILE | 10 | −4.748 | 14.701 | 8.469 | 1.00 | 0.00 | 0.396 | 9.82 | 4.00 |
| ATOM | 121 | O | ILE | 10 | −3.702 | 14.212 | 8.898 | 1.00 | 0.00 | −0.396 | 8.17 | −17.40 |
| ATOM | 122 | CB | ILE | 10 | −5.627 | 16.146 | 10.225 | 1.00 | 0.00 | −0.053 | 9.40 | 4.00 |
| ATOM | 123 | HB | ILE | 10 | −4.826 | 15.887 | 10.918 | 1.00 | 0.00 | 0.053 | 0.00 | 0.00 |
| ATOM | 124 | CG1 | ILE | 10 | −6.860 | 16.478 | 11.081 | 1.00 | 0.00 | −0.106 | 12.77 | 4.00 |
| ATOM | 125 | HG1 | ILE | 10 | −7.723 | 16.560 | 10.421 | 1.00 | 0.00 | 0.053 | 0.00 | 0.00 |
| ATOM | 126 | HG1 | ILE | 10 | −6.678 | 17.422 | 11.593 | 1.00 | 0.00 | 0.053 | 0.00 | 0.00 |
| ATOM | 127 | CG2 | ILE | 10 | −5.199 | 17.293 | 9.295 | 1.00 | 0.00 | −0.159 | 16.15 | 4.00 |
| ATOM | 128 | HG2 | ILE | 10 | −4.986 | 18.182 | 9.888 | 1.00 | 0.00 | 0.053 | 0.00 | 0.00 |
| ATOM | 129 | HG2 | ILE | 10 | −4.304 | 17.001 | 8.745 | 1.00 | 0.00 | 0.053 | 0.00 | 0.00 |
| ATOM | 130 | HG2 | ILE | 10 | −6.002 | 17.509 | 8.591 | 1.00 | 0.00 | 0.053 | 0.00 | 0.00 |
| ATOM | 131 | CD1 | ILE | 10 | −7.182 | 15.427 | 12.142 | 1.00 | 0.00 | −0.159 | 16.15 | 4.00 |
| ATOM | 132 | HD1 | ILE | 10 | −8.064 | 15.734 | 12.703 | 1.00 | 0.00 | 0.053 | 0.00 | 0.00 |
| ATOM | 133 | HD1 | ILE | 10 | −7.375 | 14.469 | 11.658 | 1.00 | 0.00 | 0.053 | 0.00 | 0.00 |
| ATOM | 134 | HD1 | ILE | 10 | −6.336 | 15.326 | 12.822 | 1.00 | 0.00 | 0.053 | 0.00 | 0.00 |
| ATOM | 135 | N | ASN | 11 | −4.919 | 15.015 | 7.167 | 1.00 | 0.00 | −0.650 | 9.00 | −17.40 |
| ATOM | 136 | HN | ASN | 11 | −5.804 | 15.440 | 6.857 | 1.00 | 0.00 | 0.440 | 0.00 | 0.00 |
| ATOM | 137 | CA | ASN | 11 | −3.879 | 14.762 | 6.208 | 1.00 | 0.00 | 0.158 | 9.40 | 4.00 |
| ATOM | 138 | HA | ASN | 11 | −2.944 | 14.632 | 6.754 | 1.00 | 0.00 | 0.053 | 0.00 | 0.00 |
| ATOM | 139 | C | ASN | 11 | −3.805 | 15.942 | 5.288 | 1.00 | 0.00 | 0.396 | 9.82 | 4.00 |
| ATOM | 140 | O | ASN | 11 | −4.642 | 16.843 | 5.348 | 1.00 | 0.00 | −0.396 | 8.17 | −17.40 |
| ATOM | 141 | CB | ASN | 11 | −4.189 | 13.579 | 5.269 | 1.00 | 0.00 | −0.106 | 12.77 | 4.00 |
| ATOM | 142 | HB1 | ASN | 11 | −3.371 | 13.416 | 4.566 | 1.00 | 0.00 | 0.053 | 0.00 | 0.00 |
| ATOM | 143 | HB2 | ASN | 11 | −5.095 | 13.766 | 4.694 | 1.00 | 0.00 | 0.053 | 0.00 | 0.00 |
| ATOM | 144 | CG | ASN | 11 | −4.391 | 12.296 | 6.062 | 1.00 | 0.00 | 0.396 | 9.82 | 4.00 |
| ATOM | 145 | OD1 | ASN | 11 | −4.277 | 12.273 | 7.286 | 1.00 | 0.00 | −0.396 | 8.17 | −17.40 |
| ATOM | 146 | ND2 | ASN | 11 | −4.704 | 11.186 | 5.342 | 1.00 | 0.00 | −0.879 | 13.25 | −17.40 |
| ATOM | 147 | HD2 | ASN | 11 | −4.790 | 11.244 | 4.317 | 1.00 | 0.00 | 0.440 | 0.00 | 0.00 |
| ATOM | 148 | HD2 | ASN | 11 | −4.854 | 10.286 | 5.821 | 1.00 | 0.00 | 0.440 | 0.00 | 0.00 |
| ATOM | 149 | N | CYS | 12 | −2.768 | 15.981 | 4.420 | 1.00 | 0.00 | −0.650 | 9.00 | −17.40 |
| ATOM | 150 | HN | CYS | 12 | −2.010 | 15.285 | 4.478 | 1.00 | 0.00 | 0.440 | 0.00 | 0.00 |
| ATOM | 151 | CA | CYS | 12 | −2.740 | 17.006 | 3.417 | 1.00 | 0.00 | 0.158 | 9.40 | 4.00 |
| ATOM | 152 | HA | CYS | 12 | −3.714 | 17.023 | 2.929 | 1.00 | 0.00 | 0.053 | 0.00 | 0.00 |
| ATOM | 153 | C | CYS | 12 | −1.656 | 16.664 | 2.441 | 1.00 | 0.00 | 0.396 | 9.82 | 4.00 |
| ATOM | 154 | O | CYS | 12 | −1.627 | 15.575 | 1.871 | 1.00 | 0.00 | −0.396 | 8.17 | −17.40 |
| ATOM | 155 | CB | CYS | 12 | −2.417 | 18.421 | 3.912 | 1.00 | 0.00 | −0.041 | 12.77 | 4.00 |
| ATOM | 156 | HB1 | CYS | 12 | −1.342 | 18.605 | 3.902 | 1.00 | 0.00 | 0.053 | 0.00 | 0.00 |
| ATOM | 157 | HB2 | CYS | 12 | −2.770 | 18.571 | 4.931 | 1.00 | 0.00 | 0.053 | 0.00 | 0.00 |
| ATOM | 158 | SG | CYS | 12 | −3.206 | 19.683 | 2.859 | 1.00 | 0.00 | −0.065 | 19.93 | −6.40 |
| ATOM | 159 | N | THR | 13 | −0.745 | 17.632 | 2.223 | 1.00 | 0.00 | −0.650 | 9.00 | −17.40 |
| ATOM | 160 | HN | THR | 13 | −0.843 | 18.500 | 2.767 | 1.00 | 0.00 | 0.440 | 0.00 | 0.00 |
| ATOM | 161 | CA | THR | 13 | 0.349 | 17.560 | 1.300 | 1.00 | 0.00 | 0.158 | 9.40 | 4.00 |
| ATOM | 162 | HA | THR | 13 | −0.031 | 17.212 | 0.339 | 1.00 | 0.00 | 0.053 | 0.00 | 0.00 |
| ATOM | 163 | C | THR | 13 | 1.354 | 16.608 | 1.834 | 1.00 | 0.00 | 0.396 | 9.82 | 4.00 |
| ATOM | 164 | O | THR | 13 | 2.087 | 15.978 | 1.075 | 1.00 | 0.00 | −0.396 | 8.17 | −17.40 |
| ATOM | 165 | CB | THR | 13 | 1.032 | 18.883 | 1.112 | 1.00 | 0.00 | 0.060 | 9.40 | 4.00 |
| ATOM | 166 | HB | THR | 13 | 0.282 | 19.629 | 0.847 | 1.00 | 0.00 | 0.053 | 0.00 | 0.00 |
| ATOM | 167 | OG1 | THR | 13 | 1.982 | 18.803 | 0.060 | 1.00 | 0.00 | −0.537 | 11.04 | −17.40 |
| ATOM | 168 | HG1 | THR | 13 | 2.157 | 19.745 | −0.317 | 1.00 | 0.00 | 0.424 | 0.00 | 0.00 |
| ATOM | 169 | CG2 | THR | 13 | 1.726 | 19.271 | 2.427 | 1.00 | 0.00 | −0.159 | 16.15 | 4.00 |
| ATOM | 170 | HG2 | THR | 13 | 2.226 | 20.231 | 2.304 | 1.00 | 0.00 | 0.053 | 0.00 | 0.00 |
| ATOM | 171 | HG2 | THR | 13 | 0.983 | 19.347 | 3.221 | 1.00 | 0.00 | 0.053 | 0.00 | 0.00 |
| ATOM | 172 | HG2 | THR | 13 | 2.460 | 18.509 | 2.689 | 1.00 | 0.00 | 0.053 | 0.00 | 0.00 |
| ATOM | 173 | N | LYS | 14 | 1.392 | 16.511 | 3.176 | 1.00 | 0.00 | −0.650 | 9.00 | −17.40 |
| ATOM | 174 | HN | LYS | 14 | 0.691 | 17.050 | 3.704 | 1.00 | 0.00 | 0.440 | 0.00 | 0.00 |
| ATOM | 175 | CA | LYS | 14 | 2.317 | 15.727 | 3.936 | 1.00 | 0.00 | 0.158 | 9.40 | 4.00 |
| ATOM | 176 | HA | LYS | 14 | 3.270 | 16.250 | 4.006 | 1.00 | 0.00 | 0.053 | 0.00 | 0.00 |
| ATOM | 177 | C | LYS | 14 | 2.527 | 14.411 | 3.268 | 1.00 | 0.00 | 0.396 | 9.82 | 4.00 |
| ATOM | 178 | O | LYS | 14 | 1.760 | 13.468 | 3.452 | 1.00 | 0.00 | −0.396 | 8.17 | −17.40 |
| ATOM | 179 | CB | LYS | 14 | 1.790 | 15.476 | 5.357 | 1.00 | 0.00 | −0.106 | 12.77 | 4.00 |
| ATOM | 180 | HB1 | LYS | 14 | 2.484 | 14.884 | 5.954 | 1.00 | 0.00 | 0.053 | 0.00 | 0.00 |
| ATOM | 181 | HB2 | LYS | 14 | 0.842 | 14.937 | 5.354 | 1.00 | 0.00 | 0.053 | 0.00 | 0.00 |
| ATOM | 182 | CG | LYS | 14 | 1.553 | 16.778 | 6.122 | 1.00 | 0.00 | −0.106 | 12.77 | 4.00 |
| ATOM | 183 | HG1 | LYS | 14 | 0.622 | 16.787 | 6.690 | 1.00 | 0.00 | 0.053 | 0.00 | 0.00 |
| ATOM | 184 | HG2 | LYS | 14 | 1.501 | 17.658 | 5.480 | 1.00 | 0.00 | 0.053 | 0.00 | 0.00 |
| ATOM | 185 | CD | LYS | 14 | 2.644 | 17.095 | 7.143 | 1.00 | 0.00 | −0.106 | 12.77 | 4.00 |
| ATOM | 186 | HD1 | LYS | 14 | 3.223 | 16.220 | 7.438 | 1.00 | 0.00 | 0.053 | 0.00 | 0.00 |
| ATOM | 187 | HD2 | LYS | 14 | 2.255 | 17.513 | 8.071 | 1.00 | 0.00 | 0.053 | 0.00 | 0.00 |
| ATOM | 188 | CE | LYS | 14 | 3.668 | 18.111 | 6.635 | 1.00 | 0.00 | 0.099 | 12.77 | 4.00 |
| ATOM | 189 | HE1 | LYS | 14 | 3.160 | 18.923 | 6.115 | 1.00 | 0.00 | 0.053 | 0.00 | 0.00 |
| ATOM | 190 | HE2 | LYS | 14 | 4.361 | 17.628 | 5.946 | 1.00 | 0.00 | 0.053 | 0.00 | 0.00 |
| ATOM | 191 | NZ | LYS | 14 | 4.431 | 18.670 | 7.771 | 1.00 | 0.00 | −0.045 | 13.25 | −39.20 |
| ATOM | 192 | HZ1 | LYS | 14 | 5.118 | 19.352 | 7.420 | 1.00 | 0.00 | 0.280 | 0.00 | 0.00 |
| ATOM | 193 | HZ2 | LYS | 14 | 3.784 | 19.137 | 8.422 | 1.00 | 0.00 | 0.280 | 0.00 | 0.00 |
| ATOM | 194 | HZ3 | LYS | 14 | 4.922 | 17.909 | 8.262 | 1.00 | 0.00 | 0.280 | 0.00 | 0.00 |
| ATOM | 195 | N | VAL | 15 | 3.595 | 14.337 | 2.452 | 1.00 | 0.00 | −0.650 | 9.00 | −17.40 |
| ATOM | 196 | HN | VAL | 15 | 4.184 | 15.170 | 2.315 | 1.00 | 0.00 | 0.440 | 0.00 | 0.00 |
| ATOM | 197 | CA | VAL | 15 | 3.927 | 13.126 | 1.771 | 1.00 | 0.00 | 0.158 | 9.40 | 4.00 |
| ATOM | 198 | HA | VAL | 15 | 3.091 | 12.448 | 1.948 | 1.00 | 0.00 | 0.053 | 0.00 | 0.00 |
| ATOM | 199 | C | VAL | 15 | 5.198 | 12.653 | 2.385 | 1.00 | 0.00 | 0.396 | 9.82 | 4.00 |
| ATOM | 200 | O | VAL | 15 | 6.097 | 13.444 | 2.663 | 1.00 | 0.00 | −0.396 | 8.17 | −17.40 |
| ATOM | 201 | CB | VAL | 15 | 4.196 | 13.312 | 0.305 | 1.00 | 0.00 | −0.053 | 9.40 | 4.00 |
| ATOM | 202 | HB | VAL | 15 | 4.506 | 12.357 | −0.119 | 1.00 | 0.00 | 0.053 | 0.00 | 0.00 |
| ATOM | 203 | CG1 | VAL | 15 | 2.907 | 13.800 | −0.378 | 1.00 | 0.00 | −0.159 | 16.15 | 4.00 |
| ATOM | 204 | HG1 | VAL | 15 | 3.089 | 13.938 | −1.443 | 1.00 | 0.00 | 0.053 | 0.00 | 0.00 |
| ATOM | 205 | HG1 | VAL | 15 | 2.118 | 13.060 | −0.238 | 1.00 | 0.00 | 0.053 | 0.00 | 0.00 |
| ATOM | 206 | HG1 | VAL | 15 | 2.597 | 14.747 | 0.063 | 1.00 | 0.00 | 0.053 | 0.00 | 0.00 |
| ATOM | 207 | CG2 | VAL | 15 | 5.385 | 14.275 | 0.145 | 1.00 | 0.00 | −0.159 | 16.15 | 4.00 |
| ATOM | 208 | HG2 | VAL | 15 | 5.594 | 14.421 | −0.914 | 1.00 | 0.00 | 0.053 | 0.00 | 0.00 |
| ATOM | 209 | HG2 | VAL | 15 | 5.140 | 15.233 | 0.602 | 1.00 | 0.00 | 0.053 | 0.00 | 0.00 |
| ATOM | 210 | HG2 | VAL | 15 | 6.263 | 13.853 | 0.633 | 1.00 | 0.00 | 0.053 | 0.00 | 0.00 |
| ATOM | 211 | N | LEU | 16 | 5.295 | 11.337 | 2.640 | 1.00 | 0.00 | −0.650 | 9.00 | −17.40 |
| ATOM | 212 | HN | LEU | 16 | 4.511 | 10.702 | 2.428 | 1.00 | 0.00 | 0.440 | 0.00 | 0.00 |
| ATOM | 213 | CA | LEU | 16 | 6.504 | 10.835 | 3.209 | 1.00 | 0.00 | 0.158 | 9.40 | 4.00 |
| ATOM | 214 | HA | LEU | 16 | 6.661 | 11.447 | 4.096 | 1.00 | 0.00 | 0.053 | 0.00 | 0.00 |
| ATOM | 215 | C | LEU | 16 | 7.528 | 11.036 | 2.145 | 1.00 | 0.00 | 0.396 | 9.82 | 4.00 |
| ATOM | 216 | O | LEU | 16 | 7.248 | 10.819 | 0.967 | 1.00 | 0.00 | −0.396 | 8.17 | −17.40 |
| ATOM | 217 | CB | LEU | 16 | 6.391 | 9.342 | 3.589 | 1.00 | 0.00 | −0.106 | 12.77 | 4.00 |
| ATOM | 218 | HB1 | LEU | 16 | 6.283 | 8.773 | 2.665 | 1.00 | 0.00 | 0.053 | 0.00 | 0.00 |
| ATOM | 219 | HB2 | LEU | 16 | 5.514 | 9.228 | 4.226 | 1.00 | 0.00 | 0.053 | 0.00 | 0.00 |
| ATOM | 220 | CG | LEU | 16 | 7.581 | 8.723 | 4.356 | 1.00 | 0.00 | −0.053 | 9.40 | 4.00 |
| ATOM | 221 | HG | LEU | 16 | 7.741 | 9.248 | 5.297 | 1.00 | 0.00 | 0.053 | 0.00 | 0.00 |
| ATOM | 222 | CD1 | LEU | 16 | 7.309 | 7.242 | 4.664 | 1.00 | 0.00 | −0.159 | 16.15 | 4.00 |
| ATOM | 223 | HD1 | LEU | 16 | 8.156 | 6.821 | 5.204 | 1.00 | 0.00 | 0.053 | 0.00 | 0.00 |
| ATOM | 224 | HD1 | LEU | 16 | 6.410 | 7.156 | 5.275 | 1.00 | 0.00 | 0.053 | 0.00 | 0.00 |
| ATOM | 225 | HD1 | LEU | 16 | 7.166 | 6.696 | 3.731 | 1.00 | 0.00 | 0.053 | 0.00 | 0.00 |
| ATOM | 226 | CD2 | LEU | 16 | 8.922 | 8.904 | 3.626 | 1.00 | 0.00 | −0.159 | 16.15 | 4.00 |
| ATOM | 227 | HD2 | LEU | 16 | 9.720 | 8.449 | 4.212 | 1.00 | 0.00 | 0.053 | 0.00 | 0.00 |
| ATOM | 228 | HD2 | LEU | 16 | 8.871 | 8.424 | 2.648 | 1.00 | 0.00 | 0.053 | 0.00 | 0.00 |
| ATOM | 229 | HD2 | LEU | 16 | 9.126 | 9.967 | 3.498 | 1.00 | 0.00 | 0.053 | 0.00 | 0.00 |
| ATOM | 230 | N | GLN | 17 | 8.735 | 11.492 | 2.528 | 1.00 | 0.00 | −0.650 | 9.00 | −17.40 |
| ATOM | 231 | HN | GLN | 17 | 8.930 | 11.656 | 3.525 | 1.00 | 0.00 | 0.440 | 0.00 | 0.00 |
| ATOM | 232 | CA | GLN | 17 | 9.747 | 11.748 | 1.549 | 1.00 | 0.00 | 0.158 | 9.40 | 4.00 |
| ATOM | 233 | HA | GLN | 17 | 9.389 | 12.551 | 0.904 | 1.00 | 0.00 | 0.053 | 0.00 | 0.00 |
| ATOM | 234 | C | GLN | 17 | 9.958 | 10.492 | 0.776 | 1.00 | 0.00 | 0.396 | 9.82 | 4.00 |
| ATOM | 235 | O | GLN | 17 | 10.347 | 9.461 | 1.323 | 1.00 | 0.00 | −0.396 | 8.17 | −17.40 |
| ATOM | 236 | CB | GLN | 17 | 11.095 | 12.186 | 2.148 | 1.00 | 0.00 | −0.106 | 12.77 | 4.00 |
| ATOM | 237 | HB1 | GLN | 17 | 11.533 | 11.448 | 2.820 | 1.00 | 0.00 | 0.053 | 0.00 | 0.00 |
| ATOM | 238 | HB2 | GLN | 17 | 11.034 | 13.103 | 2.733 | 1.00 | 0.00 | 0.053 | 0.00 | 0.00 |
| ATOM | 239 | CG | GLN | 17 | 12.173 | 12.456 | 1.096 | 1.00 | 0.00 | −0.106 | 12.77 | 4.00 |
| ATOM | 240 | HG1 | GLN | 17 | 11.813 | 13.254 | 0.447 | 1.00 | 0.00 | 0.053 | 0.00 | 0.00 |
| ATOM | 241 | HG2 | GLN | 17 | 12.329 | 11.535 | 0.533 | 1.00 | 0.00 | 0.053 | 0.00 | 0.00 |
| ATOM | 242 | CD | GLN | 17 | 13.442 | 12.873 | 1.825 | 1.00 | 0.00 | 0.396 | 9.82 | 4.00 |
| ATOM | 243 | OE1 | GLN | 17 | 14.493 | 13.066 | 1.215 | 1.00 | 0.00 | −0.396 | 8.17 | −17.40 |
| ATOM | 244 | NE2 | GLN | 17 | 13.344 | 13.021 | 3.173 | 1.00 | 0.00 | −0.879 | 13.25 | −17.40 |
| ATOM | 245 | HE2 | GLN | 17 | 12.445 | 12.849 | 3.645 | 1.00 | 0.00 | 0.440 | 0.00 | 0.00 |
| ATOM | 246 | HE2 | GLN | 17 | 14.168 | 13.304 | 3.721 | 1.00 | 0.00 | 0.440 | 0.00 | 0.00 |
| ATOM | 247 | N | GLY | 18 | 9.676 | 10.561 | −0.538 | 1.00 | 0.00 | −0.650 | 9.00 | −17.40 |
| ATOM | 248 | HN | GLY | 18 | 9.360 | 11.454 | −0.942 | 1.00 | 0.00 | 0.440 | 0.00 | 0.00 |
| ATOM | 249 | CA | GLY | 18 | 9.808 | 9.415 | −1.381 | 1.00 | 0.00 | 0.105 | 9.40 | 4.00 |
| ATOM | 250 | HA1 | GLY | 18 | 9.994 | 8.567 | −0.721 | 1.00 | 0.00 | 0.053 | 0.00 | 0.00 |
| ATOM | 251 | HA2 | GLY | 18 | 10.648 | 9.616 | −2.044 | 1.00 | 0.00 | 0.053 | 0.00 | 0.00 |
| ATOM | 252 | C | GLY | 18 | 8.520 | 9.286 | −2.120 | 1.00 | 0.00 | 0.396 | 9.82 | 4.00 |
| ATOM | 253 | O | GLY | 18 | 7.603 | 10.084 | −1.937 | 1.00 | 0.00 | −0.396 | 8.17 | −17.40 |
| ATOM | 254 | N | ASP | 19 | 8.421 | 8.260 | −2.983 | 1.00 | 0.00 | −0.650 | 9.00 | −17.40 |
| ATOM | 255 | HN | ASP | 19 | 9.213 | 7.613 | −3.103 | 1.00 | 0.00 | 0.440 | 0.00 | 0.00 |
| ATOM | 256 | CA | ASP | 19 | 7.220 | 8.062 | −3.735 | 1.00 | 0.00 | 0.158 | 9.40 | 4.00 |
| ATOM | 257 | HA | ASP | 19 | 7.030 | 8.982 | −4.286 | 1.00 | 0.00 | 0.053 | 0.00 | 0.00 |
| ATOM | 258 | C | ASP | 19 | 6.133 | 7.771 | −2.759 | 1.00 | 0.00 | 0.396 | 9.82 | 4.00 |
| ATOM | 259 | O | ASP | 19 | 5.016 | 8.272 | −2.886 | 1.00 | 0.00 | −0.396 | 8.17 | −17.40 |
| ATOM | 260 | CB | ASP | 19 | 7.308 | 6.867 | −4.700 | 1.00 | 0.00 | −0.336 | 12.77 | 4.00 |
| ATOM | 261 | HB1 | ASP | 19 | 6.308 | 6.676 | −5.090 | 1.00 | 0.00 | 0.053 | 0.00 | 0.00 |
| ATOM | 262 | HB2 | ASP | 19 | 7.677 | 6.008 | −4.138 | 1.00 | 0.00 | 0.053 | 0.00 | 0.00 |
| ATOM | 263 | CG | ASP | 19 | 8.268 | 7.239 | −5.820 | 1.00 | 0.00 | 0.297 | 9.82 | 4.00 |
| ATOM | 264 | OD1 | ASP | 19 | 8.610 | 8.447 | −5.927 | 1.00 | 0.00 | −0.534 | 8.17 | −18.95 |
| ATOM | 265 | OD2 | ASP | 19 | 8.675 | 6.322 | −6.582 | 1.00 | 0.00 | −0.534 | 8.17 | −18.95 |
| ATOM | 266 | N | VAL | 20 | 6.447 | 6.958 | −1.735 | 1.00 | 0.00 | −0.650 | 9.00 | −17.40 |
| ATOM | 267 | HN | VAL | 20 | 7.407 | 6.596 | −1.644 | 1.00 | 0.00 | 0.440 | 0.00 | 0.00 |
| ATOM | 268 | CA | VAL | 20 | 5.456 | 6.592 | −0.771 | 1.00 | 0.00 | 0.158 | 9.40 | 4.00 |
| ATOM | 269 | HA | VAL | 20 | 4.675 | 6.025 | −1.278 | 1.00 | 0.00 | 0.053 | 0.00 | 0.00 |
| ATOM | 270 | C | VAL | 20 | 4.895 | 7.838 | −0.177 | 1.00 | 0.00 | 0.396 | 9.82 | 4.00 |
| ATOM | 271 | O | VAL | 20 | 5.556 | 8.874 | −0.117 | 1.00 | 0.00 | −0.396 | 8.17 | −17.40 |
| ATOM | 272 | CB | VAL | 20 | 5.996 | 5.762 | 0.357 | 1.00 | 0.00 | −0.053 | 9.40 | 4.00 |
| ATOM | 273 | HB | VAL | 20 | 5.190 | 5.571 | 1.066 | 1.00 | 0.00 | 0.053 | 0.00 | 0.00 |
| ATOM | 274 | CG1 | VAL | 20 | 6.526 | 4.437 | −0.217 | 1.00 | 0.00 | −0.159 | 16.15 | 4.00 |
| ATOM | 275 | HG1 | VAL | 20 | 6.922 | 3.822 | 0.591 | 1.00 | 0.00 | 0.053 | 0.00 | 0.00 |
| ATOM | 276 | HG1 | VAL | 20 | 5.714 | 3.904 | −0.712 | 1.00 | 0.00 | 0.053 | 0.00 | 0.00 |
| ATOM | 277 | HG1 | VAL | 20 | 7.317 | 4.643 | −0.937 | 1.00 | 0.00 | 0.053 | 0.00 | 0.00 |
| ATOM | 278 | CG2 | VAL | 20 | 7.054 | 6.588 | 1.107 | 1.00 | 0.00 | −0.159 | 16.15 | 4.00 |
| ATOM | 279 | HG2 | VAL | 20 | 7.457 | 5.999 | 1.931 | 1.00 | 0.00 | 0.053 | 0.00 | 0.00 |
| ATOM | 280 | HG2 | VAL | 20 | 7.859 | 6.854 | 0.422 | 1.00 | 0.00 | 0.053 | 0.00 | 0.00 |
| ATOM | 281 | HG2 | VAL | 20 | 6.595 | 7.495 | 1.499 | 1.00 | 0.00 | 0.053 | 0.00 | 0.00 |
| ATOM | 282 | N | ASN | 21 | 3.625 | 7.757 | 0.261 | 1.00 | 0.00 | −0.650 | 9.00 | −17.40 |
| ATOM | 283 | HN | ASN | 21 | 3.112 | 6.871 | 0.144 | 1.00 | 0.00 | 0.440 | 0.00 | 0.00 |
| ATOM | 284 | CA | ASN | 21 | 2.967 | 8.861 | 0.882 | 1.00 | 0.00 | 0.158 | 9.40 | 4.00 |
| ATOM | 285 | HA | ASN | 21 | 3.577 | 9.730 | 0.639 | 1.00 | 0.00 | 0.053 | 0.00 | 0.00 |
| ATOM | 286 | C | ASN | 21 | 2.950 | 8.537 | 2.336 | 1.00 | 0.00 | 0.396 | 9.82 | 4.00 |
| ATOM | 287 | O | ASN | 21 | 2.614 | 7.421 | 2.729 | 1.00 | 0.00 | −0.396 | 8.17 | −17.40 |
| ATOM | 288 | CB | ASN | 21 | 1.508 | 9.014 | 0.415 | 1.00 | 0.00 | −0.106 | 12.77 | 4.00 |
| ATOM | 289 | HB1 | ASN | 21 | 0.901 | 8.224 | 0.858 | 1.00 | 0.00 | 0.053 | 0.00 | 0.00 |
| ATOM | 290 | HB2 | ASN | 21 | 1.466 | 8.939 | −0.671 | 1.00 | 0.00 | 0.053 | 0.00 | 0.00 |
| ATOM | 291 | CG | ASN | 21 | 0.978 | 10.370 | 0.853 | 1.00 | 0.00 | 0.396 | 9.82 | 4.00 |
| ATOM | 292 | OD1 | ASN | 21 | −0.156 | 10.731 | 0.541 | 1.00 | 0.00 | −0.396 | 8.17 | −17.40 |
| ATOM | 293 | ND2 | ASN | 21 | 1.819 | 11.148 | 1.585 | 1.00 | 0.00 | −0.879 | 13.25 | −17.40 |
| ATOM | 294 | HD2 | ASN | 21 | 2.760 | 10.805 | 1.823 | 1.00 | 0.00 | 0.440 | 0.00 | 0.00 |
| ATOM | 295 | HD2 | ASN | 21 | 1.514 | 12.079 | 1.900 | 1.00 | 0.00 | 0.440 | 0.00 | 0.00 |
| ATOM | 296 | N | GLU | 22 | 3.337 | 9.508 | 3.178 | 1.00 | 0.00 | −0.650 | 9.00 | −17.40 |
| ATOM | 297 | HN | GLU | 22 | 3.640 | 10.419 | 2.805 | 1.00 | 0.00 | 0.440 | 0.00 | 0.00 |
| ATOM | 298 | CA | GLU | 22 | 3.330 | 9.282 | 4.589 | 1.00 | 0.00 | 0.158 | 9.40 | 4.00 |
| ATOM | 299 | HA | GLU | 22 | 3.936 | 8.392 | 4.759 | 1.00 | 0.00 | 0.053 | 0.00 | 0.00 |
| ATOM | 300 | C | GLU | 22 | 1.903 | 9.081 | 4.974 | 1.00 | 0.00 | 0.396 | 9.82 | 4.00 |
| ATOM | 301 | O | GLU | 22 | 1.594 | 8.392 | 5.945 | 1.00 | 0.00 | −0.396 | 8.17 | −17.40 |
| ATOM | 302 | CB | GLU | 22 | 3.979 | 10.424 | 5.394 | 1.00 | 0.00 | −0.106 | 12.77 | 4.00 |
| ATOM | 303 | HB1 | GLU | 22 | 5.038 | 10.453 | 5.140 | 1.00 | 0.00 | 0.053 | 0.00 | 0.00 |
| ATOM | 304 | HB2 | GLU | 22 | 3.837 | 10.211 | 6.453 | 1.00 | 0.00 | 0.053 | 0.00 | 0.00 |
| ATOM | 305 | CG | GLU | 22 | 3.414 | 11.818 | 5.135 | 1.00 | 0.00 | −0.106 | 12.77 | 4.00 |
| ATOM | 306 | HG1 | GLU | 22 | 2.425 | 11.839 | 5.592 | 1.00 | 0.00 | 0.053 | 0.00 | 0.00 |
| ATOM | 307 | HG2 | GLU | 22 | 3.372 | 11.937 | 4.052 | 1.00 | 0.00 | 0.053 | 0.00 | 0.00 |
| ATOM | 308 | CD | GLU | 22 | 4.376 | 12.801 | 5.789 | 1.00 | 0.00 | 0.399 | 9.82 | 4.00 |
| ATOM | 309 | OE1 | GLU | 22 | 5.429 | 12.340 | 6.304 | 1.00 | 0.00 | −0.396 | 8.17 | −18.95 |
| ATOM | 310 | OE2 | GLU | 22 | 4.078 | 14.025 | 5.778 | 1.00 | 0.00 | −0.427 | 8.17 | −18.95 |
| ATOM | 312 | N | ILE | 23 | 0.996 | 9.677 | 4.182 | 1.00 | 0.00 | −0.650 | 9.00 | −17.40 |
| ATOM | 313 | HN | ILE | 23 | 1.332 | 10.253 | 3.397 | 1.00 | 0.00 | 0.440 | 0.00 | 0.00 |
| ATOM | 314 | CA | ILE | 23 | −0.416 | 9.548 | 4.378 | 1.00 | 0.00 | 0.158 | 9.40 | 4.00 |
| ATOM | 315 | HA | ILE | 23 | −0.610 | 9.980 | 5.359 | 1.00 | 0.00 | 0.053 | 0.00 | 0.00 |
| ATOM | 316 | C | ILE | 23 | −0.690 | 8.080 | 4.313 | 1.00 | 0.00 | 0.396 | 9.82 | 4.00 |
| ATOM | 317 | O | ILE | 23 | −1.640 | 7.594 | 4.918 | 1.00 | 0.00 | −0.396 | 8.17 | −17.40 |
| ATOM | 318 | CB | ILE | 23 | −1.206 | 10.137 | 3.240 | 1.00 | 0.00 | −0.053 | 9.40 | 4.00 |
| ATOM | 319 | HB | ILE | 23 | −0.853 | 9.663 | 2.323 | 1.00 | 0.00 | 0.053 | 0.00 | 0.00 |
| ATOM | 320 | CG1 | ILE | 23 | −0.924 | 11.636 | 3.040 | 1.00 | 0.00 | −0.106 | 12.77 | 4.00 |
| ATOM | 321 | HG1 | ILE | 23 | −1.404 | 11.954 | 2.114 | 1.00 | 0.00 | 0.053 | 0.00 | 0.00 |
| ATOM | 322 | HG1 | ILE | 23 | 0.155 | 11.776 | 2.980 | 1.00 | 0.00 | 0.053 | 0.00 | 0.00 |
| ATOM | 323 | CG2 | ILE | 23 | −2.688 | 9.825 | 3.505 | 1.00 | 0.00 | −0.159 | 16.15 | 4.00 |
| ATOM | 324 | HG2 | ILE | 23 | −3.296 | 10.236 | 2.699 | 1.00 | 0.00 | 0.053 | 0.00 | 0.00 |
| ATOM | 325 | HG2 | ILE | 23 | −2.829 | 8.745 | 3.551 | 1.00 | 0.00 | 0.053 | 0.00 | 0.00 |
| ATOM | 326 | HG2 | ILE | 23 | −2.990 | 10.271 | 4.452 | 1.00 | 0.00 | 0.053 | 0.00 | 0.00 |
| ATOM | 327 | CD1 | ILE | 23 | −1.446 | 12.526 | 4.163 | 1.00 | 0.00 | −0.159 | 16.15 | 4.00 |
| ATOM | 328 | HD1 | ILE | 23 | −1.205 | 13.566 | 3.944 | 1.00 | 0.00 | 0.053 | 0.00 | 0.00 |
| ATOM | 329 | HD1 | ILE | 23 | −2.527 | 12.412 | 4.244 | 1.00 | 0.00 | 0.053 | 0.00 | 0.00 |
| ATOM | 330 | HD1 | ILE | 23 | −0.978 | 12.236 | 5.104 | 1.00 | 0.00 | 0.053 | 0.00 | 0.00 |
| ATOM | 331 | N | GLN | 24 | 0.094 | 7.360 | 3.489 | 1.00 | 0.00 | −0.650 | 9.00 | −17.40 |
| ATOM | 332 | HN | GLN | 24 | 0.817 | 7.860 | 2.953 | 1.00 | 0.00 | 0.440 | 0.00 | 0.00 |
| ATOM | 333 | CA | GLN | 24 | −0.012 | 5.941 | 3.307 | 1.00 | 0.00 | 0.158 | 9.40 | 4.00 |
| ATOM | 334 | HA | GLN | 24 | −1.044 | 5.625 | 3.160 | 1.00 | 0.00 | 0.053 | 0.00 | 0.00 |
| ATOM | 335 | C | GLN | 24 | 0.514 | 5.180 | 4.496 | 1.00 | 0.00 | 0.396 | 9.82 | 4.00 |
| ATOM | 336 | O | GLN | 24 | −0.042 | 4.135 | 4.836 | 1.00 | 0.00 | −0.396 | 8.17 | −17.40 |
| ATOM | 337 | CB | GLN | 24 | 0.740 | 5.457 | 2.056 | 1.00 | 0.00 | −0.106 | 12.77 | 4.00 |
| ATOM | 338 | HB1 | GLN | 24 | 0.724 | 4.372 | 1.952 | 1.00 | 0.00 | 0.053 | 0.00 | 0.00 |
| ATOM | 339 | HB2 | GLN | 24 | 1.790 | 5.747 | 2.061 | 1.00 | 0.00 | 0.053 | 0.00 | 0.00 |
| ATOM | 340 | CG | GLN | 24 | 0.147 | 6.019 | 0.760 | 1.00 | 0.00 | −0.106 | 12.77 | 4.00 |
| ATOM | 341 | HG1 | GLN | 24 | 0.206 | 7.106 | 0.795 | 1.00 | 0.00 | 0.053 | 0.00 | 0.00 |
| ATOM | 342 | HG2 | GLN | 24 | −0.891 | 5.696 | 0.686 | 1.00 | 0.00 | 0.053 | 0.00 | 0.00 |
| ATOM | 343 | CD | GLN | 24 | 0.953 | 5.482 | −0.412 | 1.00 | 0.00 | 0.396 | 9.82 | 4.00 |
| ATOM | 344 | OE1 | GLN | 24 | 1.977 | 4.826 | −0.230 | 1.00 | 0.00 | −0.396 | 8.17 | −17.40 |
| ATOM | 345 | NE2 | GLN | 24 | 0.483 | 5.773 | −1.655 | 1.00 | 0.00 | −0.879 | 13.25 | −17.40 |
| ATOM | 346 | HE2 | GLN | 24 | −0.379 | 6.325 | −1.763 | 1.00 | 0.00 | 0.440 | 0.00 | 0.00 |
| ATOM | 347 | HE2 | GLN | 24 | 0.989 | 5.440 | −2.488 | 1.00 | 0.00 | 0.440 | 0.00 | 0.00 |
| ATOM | 348 | N | LYS | 25 | 1.583 | 5.705 | 5.148 | 1.00 | 0.00 | −0.650 | 9.00 | −17.40 |
| ATOM | 349 | HN | LYS | 25 | 1.885 | 6.639 | 4.836 | 1.00 | 0.00 | 0.440 | 0.00 | 0.00 |
| ATOM | 350 | CA | LYS | 25 | 2.354 | 5.118 | 6.228 | 1.00 | 0.00 | 0.158 | 9.40 | 4.00 |
| ATOM | 351 | HA | LYS | 25 | 3.275 | 4.714 | 5.808 | 1.00 | 0.00 | 0.053 | 0.00 | 0.00 |
| ATOM | 352 | C | LYS | 25 | 1.662 | 4.000 | 6.960 | 1.00 | 0.00 | 0.396 | 9.82 | 4.00 |
| ATOM | 353 | O | LYS | 25 | 2.026 | 2.844 | 6.764 | 1.00 | 0.00 | −0.396 | 8.17 | −17.40 |
| ATOM | 354 | CB | LYS | 25 | 2.887 | 6.155 | 7.237 | 1.00 | 0.00 | −0.106 | 12.77 | 4.00 |
| ATOM | 355 | HB1 | LYS | 25 | 2.094 | 6.726 | 7.720 | 1.00 | 0.00 | 0.053 | 0.00 | 0.00 |
| ATOM | 356 | HB2 | LYS | 25 | 3.546 | 6.893 | 6.781 | 1.00 | 0.00 | 0.053 | 0.00 | 0.00 |
| ATOM | 357 | CG | LYS | 25 | 3.694 | 5.524 | 8.373 | 1.00 | 0.00 | −0.106 | 12.77 | 4.00 |
| ATOM | 358 | HG1 | LYS | 25 | 4.416 | 4.832 | 7.938 | 1.00 | 0.00 | 0.053 | 0.00 | 0.00 |
| ATOM | 359 | HG2 | LYS | 25 | 3.005 | 4.993 | 9.030 | 1.00 | 0.00 | 0.053 | 0.00 | 0.00 |
| ATOM | 360 | CD | LYS | 25 | 4.466 | 6.534 | 9.222 | 1.00 | 0.00 | −0.106 | 12.77 | 4.00 |
| ATOM | 361 | HD1 | LYS | 25 | 3.846 | 7.349 | 9.595 | 1.00 | 0.00 | 0.053 | 0.00 | 0.00 |
| ATOM | 362 | HD2 | LYS | 25 | 5.282 | 7.016 | 8.685 | 1.00 | 0.00 | 0.053 | 0.00 | 0.00 |
| ATOM | 363 | CE | LYS | 25 | 5.110 | 5.917 | 10.464 | 1.00 | 0.00 | 0.099 | 12.77 | 4.00 |
| ATOM | 364 | HE1 | LYS | 25 | 4.349 | 5.434 | 11.077 | 1.00 | 0.00 | 0.053 | 0.00 | 0.00 |
| ATOM | 365 | HE2 | LYS | 25 | 5.600 | 6.691 | 11.053 | 1.00 | 0.00 | 0.053 | 0.00 | 0.00 |
| ATOM | 366 | NZ | LYS | 25 | 6.116 | 4.908 | 10.064 | 1.00 | 0.00 | −0.045 | 13.25 | −39.20 |
| ATOM | 367 | HZ1 | LYS | 25 | 6.544 | 4.497 | 10.905 | 1.00 | 0.00 | 0.280 | 0.00 | 0.00 |
| ATOM | 368 | HZ2 | LYS | 25 | 5.659 | 4.165 | 9.515 | 1.00 | 0.00 | 0.280 | 0.00 | 0.00 |
| ATOM | 369 | HZ3 | LYS | 25 | 6.845 | 5.357 | 9.492 | 1.00 | 0.00 | 0.280 | 0.00 | 0.00 |
| ATOM | 370 | N | VAL | 26 | 0.668 | 4.255 | 7.834 | 1.00 | 0.00 | −0.650 | 9.00 | −17.40 |
| ATOM | 371 | HN | VAL | 26 | 0.210 | 3.442 | 8.272 | 1.00 | 0.00 | 0.440 | 0.00 | 0.00 |
| ATOM | 372 | CA | VAL | 26 | 0.191 | 5.545 | 8.208 | 1.00 | 0.00 | 0.158 | 9.40 | 4.00 |
| ATOM | 373 | HA | VAL | 26 | 1.046 | 6.051 | 8.655 | 1.00 | 0.00 | 0.053 | 0.00 | 0.00 |
| ATOM | 374 | C | VAL | 26 | −0.912 | 5.293 | 9.173 | 1.00 | 0.00 | 0.396 | 9.82 | 4.00 |
| ATOM | 375 | O | VAL | 26 | −1.283 | 4.147 | 9.416 | 1.00 | 0.00 | −0.396 | 8.17 | −17.40 |
| ATOM | 376 | CB | VAL | 26 | −0.398 | 6.292 | 7.068 | 1.00 | 0.00 | −0.053 | 9.40 | 4.00 |
| ATOM | 377 | HB | VAL | 26 | 0.383 | 6.329 | 6.309 | 1.00 | 0.00 | 0.053 | 0.00 | 0.00 |
| ATOM | 378 | CG1 | VAL | 26 | −1.621 | 5.477 | 6.633 | 1.00 | 0.00 | −0.159 | 16.15 | 4.00 |
| ATOM | 379 | HG1 | VAL | 26 | −2.107 | 5.971 | 5.791 | 1.00 | 0.00 | 0.053 | 0.00 | 0.00 |
| ATOM | 380 | HG1 | VAL | 26 | −1.304 | 4.477 | 6.333 | 1.00 | 0.00 | 0.053 | 0.00 | 0.00 |
| ATOM | 381 | HG1 | VAL | 26 | −2.322 | 5.401 | 7.464 | 1.00 | 0.00 | 0.053 | 0.00 | 0.00 |
| ATOM | 382 | CG2 | VAL | 26 | −0.633 | 7.769 | 7.450 | 1.00 | 0.00 | −0.159 | 16.15 | 4.00 |
| ATOM | 383 | HG2 | VAL | 26 | −1.065 | 8.299 | 6.601 | 1.00 | 0.00 | 0.053 | 0.00 | 0.00 |
| ATOM | 384 | HG2 | VAL | 26 | −1.316 | 7.821 | 8.297 | 1.00 | 0.00 | 0.053 | 0.00 | 0.00 |
| ATOM | 385 | HG2 | VAL | 26 | 0.316 | 8.230 | 7.721 | 1.00 | 0.00 | 0.053 | 0.00 | 0.00 |
| ATOM | 386 | N | LYS | 27 | −1.470 | 6.377 | 9.731 | 1.00 | 0.00 | −0.650 | 9.00 | −17.40 |
| ATOM | 387 | HN | LYS | 27 | −1.081 | 7.304 | 9.506 | 1.00 | 0.00 | 0.440 | 0.00 | 0.00 |
| ATOM | 388 | CA | LYS | 27 | −2.577 | 6.298 | 10.621 | 1.00 | 0.00 | 0.158 | 9.40 | 4.00 |
| ATOM | 389 | HA | LYS | 27 | −2.180 | 5.771 | 11.489 | 1.00 | 0.00 | 0.053 | 0.00 | 0.00 |
| ATOM | 390 | C | LYS | 27 | −3.601 | 5.535 | 9.856 | 1.00 | 0.00 | 0.396 | 9.82 | 4.00 |
| ATOM | 391 | O | LYS | 27 | −4.312 | 4.694 | 10.402 | 1.00 | 0.00 | −0.396 | 8.17 | −17.40 |
| ATOM | 392 | CB | LYS | 27 | −3.158 | 7.699 | 10.882 | 1.00 | 0.00 | −0.106 | 12.77 | 4.00 |
| ATOM | 393 | HB1 | LYS | 27 | −3.596 | 8.055 | 9.949 | 1.00 | 0.00 | 0.053 | 0.00 | 0.00 |
| ATOM | 394 | HB2 | LYS | 27 | −2.339 | 8.342 | 11.203 | 1.00 | 0.00 | 0.053 | 0.00 | 0.00 |
| ATOM | 395 | CG | LYS | 27 | −4.247 | 7.801 | 11.950 | 1.00 | 0.00 | −0.106 | 12.77 | 4.00 |
| ATOM | 396 | HG1 | LYS | 27 | −5.021 | 7.039 | 11.860 | 1.00 | 0.00 | 0.053 | 0.00 | 0.00 |
| ATOM | 397 | HG2 | LYS | 27 | −4.784 | 8.749 | 11.940 | 1.00 | 0.00 | 0.053 | 0.00 | 0.00 |
| ATOM | 398 | CD | LYS | 27 | −3.723 | 7.662 | 13.378 | 1.00 | 0.00 | −0.106 | 12.77 | 4.00 |
| ATOM | 399 | HD1 | LYS | 27 | −3.727 | 6.637 | 13.750 | 1.00 | 0.00 | 0.053 | 0.00 | 0.00 |
| ATOM | 400 | HD2 | LYS | 27 | −4.294 | 8.228 | 14.113 | 1.00 | 0.00 | 0.053 | 0.00 | 0.00 |
| ATOM | 401 | CE | LYS | 27 | −2.284 | 8.134 | 13.554 | 1.00 | 0.00 | 0.099 | 12.77 | 4.00 |
| ATOM | 402 | HE1 | LYS | 27 | −2.180 | 9.151 | 13.176 | 1.00 | 0.00 | 0.053 | 0.00 | 0.00 |
| ATOM | 403 | HE2 | LYS | 27 | −1.610 | 7.477 | 13.002 | 1.00 | 0.00 | 0.053 | 0.00 | 0.00 |
| ATOM | 404 | NZ | LYS | 27 | −1.921 | 8.109 | 14.987 | 1.00 | 0.00 | −0.045 | 13.25 | −39.20 |
| ATOM | 405 | HZ1 | LYS | 27 | −0.948 | 8.428 | 15.100 | 1.00 | 0.00 | 0.280 | 0.00 | 0.00 |
| ATOM | 406 | HZ2 | LYS | 27 | −2.550 | 8.732 | 15.512 | 1.00 | 0.00 | 0.280 | 0.00 | 0.00 |
| ATOM | 407 | HZ3 | LYS | 27 | −2.010 | 7.148 | 15.347 | 1.00 | 0.00 | 0.280 | 0.00 | 0.00 |
| ATOM | 408 | N | LEU | 28 | −3.673 | 5.810 | 8.541 | 1.00 | 0.00 | −0.650 | 9.00 | −17.40 |
| ATOM | 409 | HN | LEU | 28 | −3.010 | 6.481 | 8.127 | 1.00 | 0.00 | 0.440 | 0.00 | 0.00 |
| ATOM | 410 | CA | LEU | 28 | −4.652 | 5.187 | 7.710 | 1.00 | 0.00 | 0.158 | 9.40 | 4.00 |
| ATOM | 411 | HA | LEU | 28 | −5.649 | 5.421 | 8.081 | 1.00 | 0.00 | 0.053 | 0.00 | 0.00 |
| ATOM | 412 | C | LEU | 28 | −4.454 | 3.709 | 7.726 | 1.00 | 0.00 | −0.396 | 9.82 | 4.00 |
| ATOM | 413 | O | LEU | 28 | −5.289 | 2.982 | 8.255 | 1.00 | 0.00 | −0.396 | 8.17 | −17.40 |
| ATOM | 414 | CB | LEU | 28 | −4.526 | 5.714 | 6.261 | 1.00 | 0.00 | −0.106 | 12.77 | 4.00 |
| ATOM | 415 | HB1 | LEU | 28 | −3.536 | 5.427 | 5.904 | 1.00 | 0.00 | 0.053 | 0.00 | 0.00 |
| ATOM | 416 | HB2 | LEU | 28 | −4.644 | 6.796 | 6.306 | 1.00 | 0.00 | 0.053 | 0.00 | 0.00 |
| ATOM | 417 | CG | LEU | 28 | −5.514 | 5.233 | 5.176 | 1.00 | 0.00 | −0.053 | 9.40 | 4.00 |
| ATOM | 418 | HG | LEU | 28 | −6.526 | 5.510 | 5.468 | 1.00 | 0.00 | 0.053 | 0.00 | 0.00 |
| ATOM | 419 | CD1 | LEU | 28 | −5.155 | 5.900 | 3.840 | 1.00 | 0.00 | −0.159 | 16.15 | 4.00 |
| ATOM | 420 | HD1 | LEU | 28 | −5.848 | 5.565 | 3.068 | 1.00 | 0.00 | 0.053 | 0.00 | 0.00 |
| ATOM | 421 | HD1 | LEU | 28 | −5.223 | 6.982 | 3.944 | 1.00 | 0.00 | 0.053 | 0.00 | 0.00 |
| ATOM | 422 | HD1 | LEU | 28 | −4.138 | 5.625 | 3.558 | 1.00 | 0.00 | 0.053 | 0.00 | 0.00 |
| ATOM | 423 | CD2 | LEU | 28 | −5.600 | 3.704 | 5.027 | 1.00 | 0.00 | −0.159 | 16.15 | 4.00 |
| ATOM | 424 | HD2 | LEU | 28 | −6.316 | 3.454 | 4.244 | 1.00 | 0.00 | 0.053 | 0.00 | 0.00 |
| ATOM | 425 | HD2 | LEU | 28 | −4.619 | 3.308 | 4.761 | 1.00 | 0.00 | 0.053 | 0.00 | 0.00 |
| ATOM | 426 | HD2 | LEU | 28 | −5.925 | 3.264 | 5.969 | 1.00 | 0.00 | 0.053 | 0.00 | 0.00 |
| ATOM | 427 | N | GLU | 29 | −3.318 | 3.204 | 7.212 | 1.00 | 0.00 | −0.650 | 9.00 | −17.40 |
| ATOM | 428 | HN | GLU | 29 | −2.547 | 3.812 | 6.901 | 1.00 | 0.00 | 0.440 | 0.00 | 0.00 |
| ATOM | 429 | CA | GLU | 29 | −3.233 | 1.772 | 7.118 | 1.00 | 0.00 | 0.158 | 9.40 | 4.00 |
| ATOM | 430 | HA | GLU | 29 | −4.108 | 1.366 | 6.611 | 1.00 | 0.00 | 0.053 | 0.00 | 0.00 |
| ATOM | 431 | C | GLU | 29 | −3.154 | 1.159 | 8.475 | 1.00 | 0.00 | 0.396 | 9.82 | 4.00 |
| ATOM | 432 | O | GLU | 29 | −3.870 | 0.204 | 8.772 | 1.00 | 0.00 | −0.396 | 8.17 | −17.40 |
| ATOM | 433 | CB | GLU | 29 | −2.067 | 1.245 | 6.258 | 1.00 | 0.00 | −0.106 | 12.77 | 4.00 |
| ATOM | 434 | HB1 | GLU | 29 | −2.179 | 1.653 | 5.253 | 1.00 | 0.00 | 0.053 | 0.00 | 0.00 |
| ATOM | 435 | HB2 | GLU | 29 | −2.125 | 0.156 | 6.245 | 1.00 | 0.00 | 0.053 | 0.00 | 0.00 |
| ATOM | 436 | CG | GLU | 29 | −0.666 | 1.614 | 6.738 | 1.00 | 0.00 | −0.106 | 12.77 | 4.00 |
| ATOM | 437 | HG1 | GLU | 29 | −0.572 | 1.221 | 7.750 | 1.00 | 0.00 | 0.053 | 0.00 | 0.00 |
| ATOM | 438 | HG2 | GLU | 29 | −0.605 | 2.701 | 6.709 | 1.00 | 0.00 | 0.053 | 0.00 | 0.00 |
| ATOM | 439 | CD | GLU | 29 | 0.310 | 0.956 | 5.771 | 1.00 | 0.00 | 0.399 | 9.82 | 4.00 |
| ATOM | 440 | OE1 | GLU | 29 | 0.543 | 1.536 | 4.677 | 1.00 | 0.00 | −0.396 | 8.17 | −18.95 |
| ATOM | 441 | OE2 | GLU | 29 | 0.833 | −0.139 | 6.112 | 1.00 | 0.00 | −0.427 | 8.17 | −18.95 |
| ATOM | 443 | N | ILE | 30 | −2.306 | 1.718 | 9.353 | 1.00 | 0.00 | −0.650 | 9.00 | −17.40 |
| ATOM | 444 | HN | ILE | 30 | −1.778 | 2.565 | 9.100 | 1.00 | 0.00 | 0.440 | 0.00 | 0.00 |
| ATOM | 445 | CA | ILE | 30 | −2.144 | 1.123 | 10.644 | 1.00 | 0.00 | 0.158 | 9.40 | 4.00 |
| ATOM | 446 | HA | ILE | 30 | −1.802 | 0.093 | 10.535 | 1.00 | 0.00 | 0.053 | 0.00 | 0.00 |
| ATOM | 447 | C | ILE | 30 | −3.446 | 1.132 | 11.371 | 1.00 | 0.00 | 0.396 | 9.82 | 4.00 |
| ATOM | 448 | O | ILE | 30 | −3.891 | 0.098 | 11.865 | 1.00 | 0.00 | −0.396 | 8.17 | −17.40 |
| ATOM | 449 | CB | ILE | 30 | −1.144 | 1.855 | 11.488 | 1.00 | 0.00 | −0.053 | 9.40 | 4.00 |
| ATOM | 450 | HB | ILE | 30 | −1.270 | 2.925 | 11.325 | 1.00 | 0.00 | 0.053 | 0.00 | 0.00 |
| ATOM | 451 | CG1 | ILE | 30 | 0.280 | 1.553 | 10.998 | 1.00 | 0.00 | −0.106 | 12.77 | 4.00 |
| ATOM | 452 | HG1 | ILE | 30 | 0.518 | 0.489 | 10.980 | 1.00 | 0.00 | 0.053 | 0.00 | 0.00 |
| ATOM | 453 | HG1 | ILE | 30 | 1.057 | 2.008 | 11.611 | 1.00 | 0.00 | 0.053 | 0.00 | 0.00 |
| ATOM | 454 | CG2 | ILE | 30 | −1.398 | 1.504 | 12.960 | 1.00 | 0.00 | −0.159 | 16.15 | 4.00 |
| ATOM | 455 | HG2 | ILE | 30 | −0.678 | 2.028 | 13.588 | 1.00 | 0.00 | 0.053 | 0.00 | 0.00 |
| ATOM | 456 | HG2 | ILE | 30 | −2.408 | 1.804 | 13.236 | 1.00 | 0.00 | 0.053 | 0.00 | 0.00 |
| ATOM | 457 | HG2 | ILE | 30 | −1.287 | 0.428 | 13.102 | 1.00 | 0.00 | 0.053 | 0.00 | 0.00 |
| ATOM | 458 | CD1 | ILE | 30 | 0.554 | 2.044 | 9.582 | 1.00 | 0.00 | −0.159 | 16.15 | 4.00 |
| ATOM | 459 | HD1 | ILE | 30 | 1.578 | 1.796 | 9.302 | 1.00 | 0.00 | 0.053 | 0.00 | 0.00 |
| ATOM | 460 | HD1 | ILE | 30 | −0.137 | 1.562 | 8.889 | 1.00 | 0.00 | 0.053 | 0.00 | 0.00 |
| ATOM | 461 | HD1 | ILE | 30 | 0.417 | 3.124 | 9.538 | 1.00 | 0.00 | 0.053 | 0.00 | 0.00 |
| ATOM | 462 | N | LEU | 31 | −4.114 | 2.295 | 11.440 | 1.00 | 0.00 | −0.650 | 9.00 | −17.40 |
| ATOM | 463 | HN | LEU | 31 | −3.762 | 3.140 | 10.967 | 1.00 | 0.00 | 0.440 | 0.00 | 0.00 |
| ATOM | 464 | CA | LEU | 31 | −5.331 | 2.320 | 12.192 | 1.00 | 0.00 | 0.158 | 9.40 | 4.00 |
| ATOM | 465 | HA | LEU | 31 | −5.170 | 1.942 | 13.202 | 1.00 | 0.00 | 0.053 | 0.00 | 0.00 |
| ATOM | 466 | C | LEU | 31 | −6.367 | 1.480 | 11.547 | 1.00 | 0.00 | 0.396 | 9.82 | 4.00 |
| ATOM | 467 | O | LEU | 31 | −7.063 | 0.724 | 12.224 | 1.00 | 0.00 | −0.396 | 8.17 | −17.40 |
| ATOM | 468 | CB | LEU | 31 | −5.913 | 3.721 | 12.409 | 1.00 | 0.00 | −0.106 | 12.77 | 4.00 |
| ATOM | 469 | HB1 | LEU | 31 | −6.956 | 3.683 | 12.722 | 1.00 | 0.00 | 0.053 | 0.00 | 0.00 |
| ATOM | 470 | HB2 | LEU | 31 | −5.875 | 4.321 | 11.500 | 1.00 | 0.00 | 0.053 | 0.00 | 0.00 |
| ATOM | 471 | CG | LEU | 31 | −5.149 | 4.496 | 13.490 | 1.00 | 0.00 | −0.053 | 9.40 | 4.00 |
| ATOM | 472 | HG | LEU | 31 | −5.139 | 3.951 | 14.434 | 1.00 | 0.00 | 0.053 | 0.00 | 0.00 |
| ATOM | 473 | CD1 | LEU | 31 | −3.690 | 4.736 | 13.076 | 1.00 | 0.00 | −0.159 | 16.15 | 4.00 |
| ATOM | 474 | HD1 | LEU | 31 | −3.174 | 5.287 | 13.862 | 1.00 | 0.00 | 0.053 | 0.00 | 0.00 |
| ATOM | 475 | HD1 | LEU | 31 | −3.193 | 3.778 | 12.919 | 1.00 | 0.00 | 0.053 | 0.00 | 0.00 |
| ATOM | 476 | HD1 | LEU | 31 | −3.664 | 5.313 | 12.151 | 1.00 | 0.00 | 0.053 | 0.00 | 0.00 |
| ATOM | 477 | CD2 | LEU | 31 | −5.912 | 5.765 | 13.899 | 1.00 | 0.00 | −0.159 | 16.15 | 4.00 |
| ATOM | 478 | HD2 | LEU | 31 | −5.349 | 6.296 | 14.666 | 1.00 | 0.00 | 0.053 | 0.00 | 0.00 |
| ATOM | 479 | HD2 | LEU | 31 | −6.037 | 6.410 | 13.029 | 1.00 | 0.00 | 0.053 | 0.00 | 0.00 |
| ATOM | 480 | HD2 | LEU | 31 | −6.891 | 5.490 | 14.291 | 1.00 | 0.00 | 0.053 | 0.00 | 0.00 |
| ATOM | 481 | N | THR | 32 | −6.493 | 1.562 | 10.215 | 1.00 | 0.00 | −0.650 | 9.00 | −17.40 |
| ATOM | 482 | HN | THR | 32 | −5.868 | 2.150 | 9.644 | 1.00 | 0.00 | 0.440 | 0.00 | 0.00 |
| ATOM | 483 | CA | THR | 32 | −7.538 | 0.791 | 9.633 | 1.00 | 0.00 | 0.158 | 9.40 | 4.00 |
| ATOM | 484 | HA | THR | 32 | −8.441 | 0.919 | 10.229 | 1.00 | 0.00 | 0.053 | 0.00 | 0.00 |
| ATOM | 485 | C | THR | 32 | −7.126 | −0.642 | 9.623 | 1.00 | 0.00 | 0.396 | 9.82 | 4.00 |
| ATOM | 486 | O | THR | 32 | −6.930 | −1.267 | 10.663 | 1.00 | 0.00 | −0.396 | 8.17 | −17.40 |
| ATOM | 467 | CB | THR | 32 | −7.960 | 1.241 | 8.250 | 1.00 | 0.00 | 0.060 | 9.40 | 4.00 |
| ATOM | 488 | HB | THR | 32 | −8.726 | 0.553 | 7.892 | 1.00 | 0.00 | 0.053 | 0.00 | 0.00 |
| ATOM | 489 | OG1 | THR | 32 | −6.888 | 1.210 | 7.318 | 1.00 | 0.00 | −0.537 | 11.04 | −17.40 |
| ATOM | 490 | HG1 | THR | 32 | −6.649 | 2.171 | 7.035 | 1.00 | 0.00 | 0.424 | 0.00 | 0.00 |
| ATOM | 491 | CG2 | THR | 32 | −8.514 | 2.670 | 8.365 | 1.00 | 0.00 | −0.159 | 16.15 | 4.00 |
| ATOM | 492 | HG2 | THR | 32 | −8.826 | 3.019 | 7.380 | 1.00 | 0.00 | 0.053 | 0.00 | 0.00 |
| ATOM | 493 | HG2 | THR | 32 | −9.369 | 2.675 | 9.040 | 1.00 | 0.00 | 0.053 | 0.00 | 0.00 |
| ATOM | 494 | HG2 | THR | 32 | −7.739 | 3.330 | 8.755 | 1.00 | 0.00 | 0.053 | 0.00 | 0.00 |
| ATOM | 495 | N | VAL | 33 | −6.936 | −1.186 | 8.421 | 1.00 | 0.00 | −0.650 | 9.00 | −17.40 |
| ATOM | 496 | HN | VAL | 33 | −6.941 | −0.577 | 7.590 | 1.00 | 0.00 | 0.440 | 0.00 | 0.00 |
| ATOM | 497 | CA | VAL | 33 | −6.727 | −2.579 | 8.252 | 1.00 | 0.00 | 0.158 | 9.40 | 4.00 |
| ATOM | 498 | HA | VAL | 33 | −7.581 | −3.173 | 8.574 | 1.00 | 0.00 | 0.053 | 0.00 | 0.00 |
| ATOM | 499 | C | VAL | 33 | −5.554 | −3.083 | 9.028 | 1.00 | 0.00 | 0.396 | 9.82 | 4.00 |
| ATOM | 500 | O | VAL | 33 | −5.651 | −4.156 | 9.620 | 1.00 | 0.00 | −0.396 | 8.17 | −17.40 |
| ATOM | 501 | CB | VAL | 33 | −6.484 | −2.949 | 6.817 | 1.00 | 0.00 | −0.053 | 9.40 | 4.00 |
| ATOM | 502 | HB | VAL | 33 | −7.329 | −2.602 | 6.222 | 1.00 | 0.00 | 0.053 | 0.00 | 0.00 |
| ATOM | 503 | CG1 | VAL | 33 | −5.185 | −2.269 | 6.350 | 1.00 | 0.00 | −0.159 | 16.15 | 4.00 |
| ATOM | 504 | HG1 | VAL | 33 | −4.993 | −2.527 | 5.308 | 1.00 | 0.00 | 0.053 | 0.00 | 0.00 |
| ATOM | 505 | HG1 | VAL | 33 | −5.286 | −1.187 | 6.442 | 1.00 | 0.00 | 0.053 | 0.00 | 0.00 |
| ATOM | 506 | HG1 | VAL | 33 | −4.353 | −2.609 | 6.967 | 1.00 | 0.00 | 0.053 | 0.00 | 0.00 |
| ATOM | 507 | CG2 | VAL | 33 | −6.450 | −4.483 | 6.704 | 1.00 | 0.00 | −0.159 | 16.15 | 4.00 |
| ATOM | 508 | HG2 | VAL | 33 | −6.274 | −4.767 | 5.666 | 1.00 | 0.00 | 0.053 | 0.00 | 0.00 |
| ATOM | 509 | HG2 | VAL | 33 | −5.648 | −4.876 | 7.329 | 1.00 | 0.00 | 0.053 | 0.00 | 0.00 |
| ATOM | 510 | HG2 | VAL | 33 | −7.403 | −4.893 | 7.036 | 1.00 | 0.00 | 0.053 | 0.00 | 0.00 |
| ATOM | 511 | N | LYS | 34 | −4.432 | −2.337 | 9.076 | 1.00 | 0.00 | −0.650 | 9.00 | −17.40 |
| ATOM | 512 | HN | LYS | 34 | −4.423 | −1.370 | 8.720 | 1.00 | 0.00 | 0.440 | 0.00 | 0.00 |
| ATOM | 513 | CA | LYS | 34 | −3.247 | −2.928 | 9.637 | 1.00 | 0.00 | 0.158 | 9.40 | 4.00 |
| ATOM | 514 | HA | LYS | 34 | −2.914 | −3.802 | 9.077 | 1.00 | 0.00 | 0.053 | 0.00 | 0.00 |
| ATOM | 515 | C | LYS | 34 | −3.449 | −3.381 | 11.047 | 1.00 | 0.00 | 0.396 | 9.82 | 4.00 |
| ATOM | 516 | O | LYS | 34 | −3.224 | −4.551 | 11.351 | 1.00 | 0.00 | −0.396 | 8.17 | −17.40 |
| ATOM | 517 | CB | LYS | 34 | −2.001 | −2.025 | 9.609 | 1.00 | 0.00 | −0.106 | 12.77 | 4.00 |
| ATOM | 518 | HB1 | LYS | 34 | −2.133 | −1.147 | 10.241 | 1.00 | 0.00 | 0.053 | 0.00 | 0.00 |
| ATOM | 519 | HB2 | LYS | 34 | −1.788 | −1.673 | 8.599 | 1.00 | 0.00 | 0.053 | 0.00 | 0.00 |
| ATOM | 520 | CG | LYS | 34 | −0.760 | −2.772 | 10.109 | 1.00 | 0.00 | −0.106 | 12.77 | 4.00 |
| ATOM | 521 | HG1 | LYS | 34 | −0.752 | −3.762 | 9.653 | 1.00 | 0.00 | 0.053 | 0.00 | 0.00 |
| ATOM | 522 | HG2 | LYS | 34 | −0.823 | −2.847 | 11.194 | 1.00 | 0.00 | 0.053 | 0.00 | 0.00 |
| ATOM | 523 | CD | LYS | 34 | 0.573 | −2.102 | 9.772 | 1.00 | 0.00 | −0.106 | 12.77 | 4.00 |
| ATOM | 524 | HD1 | LYS | 34 | 0.665 | −1.094 | 10.178 | 1.00 | 0.00 | 0.053 | 0.00 | 0.00 |
| ATOM | 525 | HD2 | LYS | 34 | 0.745 | −1.997 | 8.700 | 1.00 | 0.00 | 0.053 | 0.00 | 0.00 |
| ATOM | 526 | CE | LYS | 34 | 1.789 | −2.866 | 10.305 | 1.00 | 0.00 | 0.099 | 12.77 | 4.00 |
| ATOM | 527 | HE1 | LYS | 34 | 1.666 | −3.076 | 11.367 | 1.00 | 0.00 | 0.053 | 0.00 | 0.00 |
| ATOM | 528 | HE2 | LYS | 34 | 2.695 | −2.275 | 10.168 | 1.00 | 0.00 | 0.053 | 0.00 | 0.00 |
| ATOM | 529 | NZ | LYS | 34 | 1.946 | −4.148 | 9.583 | 1.00 | 0.00 | −0.045 | 13.25 | −39.20 |
| ATOM | 530 | HZ1 | LYS | 34 | 2.766 | −4.651 | 9.950 | 1.00 | 0.00 | 0.280 | 0.00 | 0.00 |
| ATOM | 531 | HZ2 | LYS | 34 | 1.102 | −4.723 | 9.716 | 1.00 | 0.00 | 0.280 | 0.00 | 0.00 |
| ATOM | 532 | HZ3 | LYS | 34 | 2.079 | −3.962 | 8.578 | 1.00 | 0.00 | 0.280 | 0.00 | 0.00 |
| ATOM | 533 | N | PHE | 35 | −3.909 | −2.515 | 11.965 | 1.00 | 0.00 | −0.650 | 9.00 | −17.40 |
| ATOM | 534 | HN | PHE | 35 | −4.135 | −1.531 | 11.759 | 1.00 | 0.00 | 0.440 | 0.00 | 0.00 |
| ATOM | 535 | CA | PHE | 35 | −4.049 | −3.108 | 13.257 | 1.00 | 0.00 | 0.158 | 9.40 | 4.00 |
| ATOM | 536 | HA | PHE | 35 | −3.229 | −3.809 | 13.414 | 1.00 | 0.00 | 0.053 | 0.00 | 0.00 |
| ATOM | 537 | C | PHE | 35 | −5.342 | −3.818 | 13.320 | 1.00 | 0.00 | 0.396 | 9.82 | 4.00 |
| ATOM | 538 | O | PHE | 35 | −6.399 | −3.228 | 13.542 | 1.00 | 0.00 | −0.396 | 8.17 | −17.40 |
| ATOM | 539 | CB | PHE | 35 | −3.786 | −2.198 | 14.464 | 1.00 | 0.00 | −0.106 | 12.77 | 4.00 |
| ATOM | 540 | HB1 | PHE | 35 | −4.265 | −2.579 | 15.365 | 1.00 | 0.00 | 0.053 | 0.00 | 0.00 |
| ATOM | 541 | HB2 | PHE | 35 | −4.166 | −1.190 | 14.296 | 1.00 | 0.00 | 0.053 | 0.00 | 0.00 |
| ATOM | 542 | CG | PHE | 35 | −2.297 | −2.243 | 14.544 | 1.00 | 0.00 | 0.000 | 7.26 | 0.60 |
| ATOM | 543 | CD1 | PHE | 35 | −1.679 | −3.358 | 15.061 | 1.00 | 0.00 | −0.127 | 10.80 | 0.60 |
| ATOM | 544 | HD1 | PHE | 35 | −2.285 | −4.192 | 15.413 | 1.00 | 0.00 | 0.127 | 0.00 | 0.00 |
| ATOM | 545 | CD2 | PHE | 35 | −1.515 | −1.207 | 14.096 | 1.00 | 0.00 | −0.127 | 10.80 | 0.60 |
| ATOM | 546 | HD2 | PHE | 35 | −1.987 | −0.319 | 13.675 | 1.00 | 0.00 | 0.127 | 0.00 | 0.00 |
| ATOM | 547 | CE1 | PHE | 35 | −0.309 | −3.437 | 15.140 | 1.00 | 0.00 | −0.127 | 10.80 | 0.60 |
| ATOM | 548 | HE1 | PHE | 35 | 0.163 | −4.325 | 15.557 | 1.00 | 0.00 | 0.127 | 0.00 | 0.00 |
| ATOM | 549 | CE2 | PHE | 35 | −0.143 | −1.275 | 14.171 | 1.00 | 0.00 | −0.127 | 10.80 | 0.60 |
| ATOM | 550 | HE2 | PHE | 35 | 0.463 | −0.441 | 13.817 | 1.00 | 0.00 | 0.127 | 0.00 | 0.00 |
| ATOM | 551 | CZ | PHE | 35 | 0.464 | −2.393 | 14.691 | 1.00 | 0.00 | −0.127 | 10.80 | 0.60 |
| ATOM | 552 | HZ | PHE | 35 | 1.551 | −2.451 | 14.747 | 1.00 | 0.00 | 0.127 | 0.00 | 0.00 |
| ATOM | 553 | N | LYS | 36 | −5.213 | −5.147 | 13.118 | 1.00 | 0.00 | −0.650 | 9.00 | −17.40 |
| ATOM | 554 | HN | LYS | 36 | −4.250 | −5.492 | 12.994 | 1.00 | 0.00 | 0.440 | 0.00 | 0.00 |
| ATOM | 555 | CA | LYS | 36 | −6.244 | −6.135 | 13.054 | 1.00 | 0.00 | 0.158 | 9.40 | 4.00 |
| ATOM | 556 | HA | LYS | 36 | −6.877 | −5.836 | 12.218 | 1.00 | 0.00 | 0.053 | 0.00 | 0.00 |
| ATOM | 557 | C | LYS | 36 | −6.931 | −6.068 | 14.362 | 1.00 | 0.00 | 0.396 | 9.82 | 4.00 |
| ATOM | 558 | O | LYS | 36 | −8.099 | −6.431 | 14.487 | 1.00 | 0.00 | −0.396 | 8.17 | −17.40 |
| ATOM | 559 | CB | LYS | 36 | −5.669 | −7.554 | 12.924 | 1.00 | 0.00 | −0.106 | 12.77 | 4.00 |
| ATOM | 560 | HB1 | LYS | 36 | −6.432 | −8.310 | 12.740 | 1.00 | 0.00 | 0.053 | 0.00 | 0.00 |
| ATOM | 561 | HB2 | LYS | 36 | −5.138 | −7.884 | 13.817 | 1.00 | 0.00 | 0.053 | 0.00 | 0.00 |
| ATOM | 562 | CG | LYS | 36 | −4.668 | −7.694 | 11.777 | 1.00 | 0.00 | −0.106 | 12.77 | 4.00 |
| ATOM | 563 | HG1 | LYS | 36 | −4.288 | −8.715 | 11.778 | 1.00 | 0.00 | 0.053 | 0.00 | 0.00 |
| ATOM | 564 | HG2 | LYS | 36 | −3.859 | −6.982 | 11.941 | 1.00 | 0.00 | 0.053 | 0.00 | 0.00 |
| ATOM | 565 | CD | LYS | 36 | −5.248 | −7.420 | 10.390 | 1.00 | 0.00 | −0.106 | 12.77 | 4.00 |
| ATOM | 566 | HD1 | LYS | 36 | −4.559 | −6.898 | 9.725 | 1.00 | 0.00 | 0.053 | 0.00 | 0.00 |
| ATOM | 567 | HD2 | LYS | 36 | −6.147 | −6.804 | 10.407 | 1.00 | 0.00 | 0.053 | 0.00 | 0.00 |
| ATOM | 568 | CE | LYS | 36 | −5.648 | −8.687 | 9.634 | 1.00 | 0.00 | 0.099 | 12.77 | 4.00 |
| ATOM | 569 | HE1 | LYS | 36 | −6.460 | −9.191 | 10.157 | 1.00 | 0.00 | 0.053 | 0.00 | 0.00 |
| ATOM | 570 | HE2 | LYS | 36 | −4.796 | −9.363 | 9.565 | 1.00 | 0.00 | 0.053 | 0.00 | 0.00 |
| ATOM | 571 | NZ | LYS | 36 | −6.097 | −8.339 | 8.268 | 1.00 | 0.00 | −0.045 | 13.25 | −39.20 |
| ATOM | 572 | HZ1 | LYS | 36 | −6.364 | −9.197 | 7.765 | 1.00 | 0.00 | 0.280 | 0.00 | 0.00 |
| ATOM | 573 | HZ2 | LYS | 36 | −6.908 | −7.706 | 8.323 | 1.00 | 0.00 | 0.280 | 0.00 | 0.00 |
| ATOM | 574 | HZ3 | LYS | 36 | −5.331 | −7.870 | 7.763 | 1.00 | 0.00 | 0.280 | 0.00 | 0.00 |
| ATOM | 575 | N | LYS | 37 | −6.190 | −5.575 | 15.370 | 1.00 | 0.00 | −0.650 | 9.00 | −17.40 |
| ATOM | 576 | HN | LYS | 37 | −5.217 | −5.294 | 15.177 | 1.00 | 0.00 | 0.440 | 0.00 | 0.00 |
| ATOM | 577 | CA | LYS | 37 | −6.697 | −5.425 | 16.696 | 1.00 | 0.00 | 0.158 | 9.40 | 4.00 |
| ATOM | 578 | HA | LYS | 37 | −6.864 | −6.438 | 17.060 | 1.00 | 0.00 | 0.053 | 0.00 | 0.00 |
| ATOM | 579 | C | LYS | 37 | −7.953 | −4.640 | 16.555 | 1.00 | 0.00 | 0.396 | 9.82 | 4.00 |
| ATOM | 580 | O | LYS | 37 | −8.889 | −4.838 | 17.327 | 1.00 | 0.00 | −0.396 | 8.17 | −17.40 |
| ATOM | 581 | CB | LYS | 37 | −5.747 | −4.621 | 17.601 | 1.00 | 0.00 | −0.106 | 12.77 | 4.00 |
| ATOM | 582 | HB1 | LYS | 37 | −5.651 | −3.616 | 17.189 | 1.00 | 0.00 | 0.053 | 0.00 | 0.00 |
| ATOM | 583 | HB2 | LYS | 37 | −4.780 | −5.124 | 17.614 | 1.00 | 0.00 | 0.053 | 0.00 | 0.00 |
| ATOM | 584 | CG | LYS | 37 | −6.218 | −4.485 | 19.051 | 1.00 | 0.00 | −0.106 | 12.77 | 4.00 |
| ATOM | 585 | HG1 | LYS | 37 | −7.246 | −4.136 | 19.145 | 1.00 | 0.00 | 0.053 | 0.00 | 0.00 |
| ATOM | 586 | HG2 | LYS | 37 | −5.627 | −3.782 | 19.638 | 1.00 | 0.00 | 0.053 | 0.00 | 0.00 |
| ATOM | 587 | CD | LYS | 37 | −6.170 | −5.796 | 19.837 | 1.00 | 0.00 | −0.106 | 12.77 | 4.00 |
| ATOM | 588 | HD1 | LYS | 37 | −5.183 | −6.259 | 19.848 | 1.00 | 0.00 | 0.053 | 0.00 | 0.00 |
| ATOM | 589 | HD2 | LYS | 37 | −6.840 | −6.561 | 19.446 | 1.00 | 0.00 | 0.053 | 0.00 | 0.00 |
| ATOM | 590 | CE | LYS | 37 | −6.556 | −5.638 | 21.309 | 1.00 | 0.00 | 0.099 | 12.77 | 4.00 |
| ATOM | 591 | HE1 | LYS | 37 | −6.610 | −6.617 | 21.784 | 1.00 | 0.00 | 0.053 | 0.00 | 0.00 |
| ATOM | 592 | HE2 | LYS | 37 | −7.527 | −5.149 | 21.387 | 1.00 | 0.00 | 0.053 | 0.00 | 0.00 |
| ATOM | 593 | NZ | LYS | 37 | −5.544 | −4.816 | 22.009 | 1.00 | 0.00 | −0.045 | 13.25 | −39.20 |
| ATOM | 594 | HZ1 | LYS | 37 | −5.809 | −4.713 | 22.998 | 1.00 | 0.00 | 0.280 | 0.00 | 0.00 |
| ATOM | 595 | HZ2 | LYS | 37 | −4.624 | −5.275 | 21.946 | 1.00 | 0.00 | 0.280 | 0.00 | 0.00 |
| ATOM | 596 | HZ3 | LYS | 37 | −5.492 | −3.885 | 21.569 | 1.00 | 0.00 | 0.280 | 0.00 | 0.00 |
| ATOM | 597 | N | ARG | 38 | −7.978 | −3.734 | 15.549 | 1.00 | 0.00 | −0.650 | 9.00 | −17.40 |
| ATOM | 598 | HN | ARG | 38 | −7.124 | −3.623 | 14.983 | 1.00 | 0.00 | 0.440 | 0.00 | 0.00 |
| ATOM | 599 | CA | ARG | 38 | −9.108 | −2.917 | 15.214 | 1.00 | 0.00 | 0.158 | 9.40 | 4.00 |
| ATOM | 600 | HA | ARG | 38 | −9.173 | −2.143 | 15.979 | 1.00 | 0.00 | 0.053 | 0.00 | 0.00 |
| ATOM | 601 | C | ARG | 38 | −10.295 | −3.807 | 15.218 | 1.00 | 0.00 | 0.396 | 9.82 | 4.00 |
| ATOM | 602 | O | ARG | 38 | −10.569 | −4.570 | 14.292 | 1.00 | 0.00 | −0.396 | 8.17 | −17.40 |
| ATOM | 603 | CB | ARG | 38 | −9.013 | −2.282 | 13.830 | 1.00 | 0.00 | −0.106 | 12.77 | 4.00 |
| ATOM | 604 | HB1 | ARG | 38 | −8.846 | −3.033 | 13.058 | 1.00 | 0.00 | 0.053 | 0.00 | 0.00 |
| ATOM | 605 | HB2 | ARG | 38 | −8.190 | −1.569 | 13.775 | 1.00 | 0.00 | 0.053 | 0.00 | 0.00 |
| ATOM | 606 | CG | ARG | 38 | −10.288 | −1.536 | 13.474 | 1.00 | 0.00 | −0.106 | 12.17 | 4.00 |
| ATOM | 607 | HG1 | ARG | 38 | −10.466 | −0.681 | 14.126 | 1.00 | 0.00 | 0.053 | 0.00 | 0.00 |
| ATOM | 608 | HG2 | ARG | 38 | −11.173 | −2.166 | 13.546 | 1.00 | 0.00 | 0.053 | 0.00 | 0.00 |
| ATOM | 609 | CD | ARG | 38 | −10.266 | −0.992 | 12.054 | 1.00 | 0.00 | 0.374 | 12.77 | 4.00 |
| ATOM | 610 | HD1 | ARG | 38 | −9.830 | 0.005 | 12.116 | 1.00 | 0.00 | 0.053 | 0.00 | 0.00 |
| ATOM | 611 | HD2 | ARG | 38 | −11.302 | −0.974 | 11.718 | 1.00 | 0.00 | 0.053 | 0.00 | 0.00 |
| ATOM | 612 | NE | ARG | 38 | −9.428 | −1.930 | 11.262 | 1.00 | 0.00 | −0.819 | 9.00 | −24.67 |
| ATOM | 613 | HE | ARG | 38 | −8.457 | −1.675 | 11.030 | 1.00 | 0.00 | 0.407 | 0.00 | 0.00 |
| ATOM | 614 | CZ | ARG | 38 | −9.932 | −3.125 | 10.839 | 1.00 | 0.00 | 0.796 | 6.95 | 4.00 |
| ATOM | 615 | NH1 | ARG | 38 | −11.203 | −3.493 | 11.171 | 1.00 | 0.00 | −0.746 | 9.00 | −24.67 |
| ATOM | 616 | HH1 | ARG | 38 | −11.578 | −4.396 | 10.848 | 1.00 | 0.00 | 0.407 | 0.00 | 0.00 |
| ATOM | 617 | HH1 | ARG | 38 | −11.787 | −2.867 | 11.743 | 1.00 | 0.00 | 0.407 | 0.00 | 0.00 |
| ATOM | 618 | NH2 | ARG | 38 | −9.136 | −3.971 | 10.124 | 1.00 | 0.00 | −0.746 | 9.00 | −24.67 |
| ATOM | 619 | HH2 | ARG | 38 | −9.506 | −4.875 | 9.799 | 1.00 | 0.00 | 0.407 | 0.00 | 0.00 |
| ATOM | 620 | HH2 | ARG | 38 | −8.164 | −3.705 | 9.908 | 1.00 | 0.00 | 0.407 | 0.00 | 0.00 |
| ATOM | 621 | N | PRO | 39 | −10.992 | −3.676 | 16.300 | 1.00 | 0.00 | −0.422 | 9.00 | −17.40 |
| ATOM | 622 | CA | PRO | 39 | −12.095 | −4.511 | 16.644 | 1.00 | 0.00 | 0.158 | 9.40 | 4.00 |
| ATOM | 623 | HA | PRO | 39 | −11.727 | −5.532 | 16.738 | 1.00 | 0.00 | 0.053 | 0.00 | 0.00 |
| ATOM | 624 | CD | PRO | 39 | −10.693 | −2.681 | 17.317 | 1.00 | 0.00 | 0.105 | 12.77 | 4.00 |
| ATOM | 625 | HD1 | PRO | 39 | −11.078 | −1.702 | 17.028 | 1.00 | 0.00 | 0.053 | 0.00 | 0.00 |
| ATOM | 626 | HD2 | PRO | 39 | −9.617 | −2.589 | 17.466 | 1.00 | 0.00 | 0.053 | 0.00 | 0.00 |
| ATOM | 627 | C | PRO | 39 | −13.210 | −4.532 | 15.662 | 1.00 | 0.00 | 0.396 | 9.82 | 4.00 |
| ATOM | 628 | O | PRO | 39 | −13.761 | −5.611 | 15.454 | 1.00 | 0.00 | −0.396 | 8.17 | −17.40 |
| ATOM | 629 | CB | PRO | 39 | −12.572 | −4.034 | 18.017 | 1.00 | 0.00 | −0.106 | 12.77 | 4.00 |
| ATOM | 630 | HB1 | PRO | 39 | −12.774 | −4.949 | 18.572 | 1.00 | 0.00 | 0.053 | 0.00 | 0.00 |
| ATOM | 631 | HB2 | PRO | 39 | −13.461 | −3.439 | 17.808 | 1.00 | 0.00 | 0.053 | 0.00 | 0.00 |
| ATOM | 632 | CG | PRO | 39 | −11.394 | −3.215 | 18.576 | 1.00 | 0.00 | −0.106 | 12.77 | 4.00 |
| ATOM | 633 | HG1 | PRO | 39 | −10.730 | −3.842 | 19.170 | 1.00 | 0.00 | 0.053 | 0.00 | 0.00 |
| ATOM | 634 | HG2 | PRO | 39 | −11.749 | −2.404 | 19.212 | 1.00 | 0.00 | 0.053 | 0.00 | 0.00 |
| ATOM | 635 | N | ARG | 40 | −13.588 | −3.400 | 15.033 | 1.00 | 0.00 | −0.650 | 9.00 | −17.40 |
| ATOM | 636 | HN | ARG | 40 | −13.092 | −2.499 | 15.095 | 1.00 | 0.00 | 0.440 | 0.00 | 0.00 |
| ATOM | 637 | CA | ARG | 40 | −14.773 | −3.627 | 14.268 | 1.00 | 0.00 | 0.158 | 9.40 | 4.00 |
| ATOM | 638 | HA | ARG | 40 | −14.758 | −4.565 | 13.713 | 1.00 | 0.00 | 0.053 | 0.00 | 0.00 |
| ATOM | 639 | C | ARG | 40 | −15.031 | −2.570 | 13.244 | 1.00 | 0.00 | 0.396 | 9.82 | 4.00 |
| ATOM | 640 | O | ARG | 40 | −14.432 | −1.498 | 13.219 | 1.00 | 0.00 | −0.396 | 8.17 | −17.40 |
| ATOM | 641 | CB | ARG | 40 | −16.020 | −3.751 | 15.168 | 1.00 | 0.00 | −0.106 | 12.77 | 4.00 |
| ATOM | 642 | HB1 | ARG | 40 | −16.266 | −2.756 | 15.541 | 1.00 | 0.00 | 0.053 | 0.00 | 0.00 |
| ATOM | 643 | HB2 | ARG | 40 | −15.777 | −4.425 | 15.989 | 1.00 | 0.00 | 0.053 | 0.00 | 0.00 |
| ATOM | 644 | CG | ARG | 40 | −17.275 | −4.303 | 14.483 | 1.00 | 0.00 | −0.106 | 12.77 | 4.00 |
| ATOM | 645 | HG1 | ARG | 40 | −17.506 | −3.668 | 13.627 | 1.00 | 0.00 | 0.053 | 0.00 | 0.00 |
| ATOM | 646 | HG2 | ARG | 40 | −18.093 | −4.288 | 15.202 | 1.00 | 0.00 | 0.053 | 0.00 | 0.00 |
| ATOM | 647 | CD | ARG | 40 | −17.127 | −5.740 | 13.972 | 1.00 | 0.00 | 0.374 | 12.77 | 4.00 |
| ATOM | 648 | HD1 | ARG | 40 | −16.628 | −5.707 | 13.003 | 1.00 | 0.00 | 0.053 | 0.00 | 0.00 |
| ATOM | 649 | HD2 | ARG | 40 | −18.123 | −6.172 | 13.878 | 1.00 | 0.00 | 0.053 | 0.00 | 0.00 |
| ATOM | 650 | NE | ARG | 40 | −16.311 | −6.505 | 14.960 | 1.00 | 0.00 | −0.819 | 9.00 | −24.67 |
| ATOM | 651 | HE | ARG | 40 | −15.286 | −6.528 | 14.855 | 1.00 | 0.00 | 0.407 | 0.00 | 0.00 |
| ATOM | 652 | CZ | ARG | 40 | −16.891 | −7.175 | 15.999 | 1.00 | 0.00 | 0.796 | 6.95 | 4.00 |
| ATOM | 653 | NH1 | ARG | 40 | −18.246 | −7.146 | 16.162 | 1.00 | 0.00 | −0.746 | 9.00 | −24.67 |
| ATOM | 654 | HH1 | ARG | 40 | −18.681 | −7.652 | 16.946 | 1.00 | 0.00 | 0.407 | 0.00 | 0.00 |
| ATOM | 655 | HH1 | ARG | 40 | −18.833 | −6.617 | 15.500 | 1.00 | 0.00 | 0.407 | 0.00 | 0.00 |
| ATOM | 656 | NH2 | ARG | 40 | −16.109 | −7.883 | 16.867 | 1.00 | 0.00 | −0.746 | 9.00 | −24.67 |
| ATOM | 657 | HH2 | ARG | 40 | −16.540 | −8.390 | 17.652 | 1.00 | 0.00 | 0.407 | 0.00 | 0.00 |
| ATOM | 658 | HH2 | ARG | 40 | −15.087 | −7.909 | 16.735 | 1.00 | 0.00 | 0.407 | 0.00 | 0.00 |
| ATOM | 659 | N | TRP | 41 | −15.994 | −2.921 | 12.378 | 1.00 | 0.00 | −0.650 | 9.00 | −17.40 |
| ATOM | 660 | HN | TRP | 41 | −16.411 | −3.841 | 12.577 | 1.00 | 0.00 | 0.440 | 0.00 | 0.00 |
| ATOM | 661 | CA | TRP | 41 | −16.559 | −2.274 | 11.232 | 1.00 | 0.00 | 0.158 | 9.40 | 4.00 |
| ATOM | 662 | HA | TRP | 41 | −15.784 | −2.112 | 10.481 | 1.00 | 0.00 | 0.053 | 0.00 | 0.00 |
| ATOM | 663 | C | TRP | 41 | −17.140 | −0.953 | 11.633 | 1.00 | 0.00 | 0.396 | 9.82 | 4.00 |
| ATOM | 664 | O | TRP | 41 | −16.851 | 0.072 | 11.016 | 1.00 | 0.00 | −0.396 | 8.17 | −17.40 |
| ATOM | 665 | CB | TRP | 41 | −17.691 | −3.219 | 10.774 | 1.00 | 0.00 | −0.106 | 12.77 | 4.00 |
| ATOM | 666 | HB1 | TRP | 41 | −18.381 | −3.292 | 11.614 | 1.00 | 0.00 | 0.053 | 0.00 | 0.00 |
| ATOM | 667 | HB2 | TRP | 41 | −17.214 | −4.170 | 10.538 | 1.00 | 0.00 | 0.053 | 0.00 | 0.00 |
| ATOM | 668 | CG | TRP | 41 | −18.571 | −2.932 | 9.584 | 1.00 | 0.00 | 0.000 | 7.26 | 0.60 |
| ATOM | 669 | CD1 | TRP | 41 | −18.592 | −1.908 | 8.684 | 1.00 | 0.00 | −0.177 | 10.80 | 0.60 |
| ATOM | 670 | HD1 | TRP | 41 | −17.912 | −1.055 | 8.688 | 1.00 | 0.00 | 0.127 | 0.00 | 0.00 |
| ATOM | 671 | CD2 | TRP | 41 | −19.625 | −3.833 | 9.203 | 1.00 | 0.00 | 0.000 | 6.80 | 0.60 |
| ATOM | 672 | NE1 | TRP | 41 | −19.601 | −2.113 | 7.769 | 1.00 | 0.00 | −0.292 | 9.00 | −17.40 |
| ATOM | 673 | HE1 | TRP | 41 | −19.838 | −1.487 | 6.986 | 1.00 | 0.00 | 0.393 | 0.00 | 0.00 |
| ATOM | 674 | CE2 | TRP | 41 | −20.242 | −3.297 | 8.076 | 1.00 | 0.00 | −0.050 | 6.80 | 0.60 |
| ATOM | 675 | CE3 | TRP | 41 | −20.044 | −5.015 | 9.750 | 1.00 | 0.00 | −0.127 | 10.80 | 0.60 |
| ATOM | 676 | HE3 | TRP | 41 | −19.561 | −5.432 | 10.633 | 1.00 | 0.00 | 0.127 | 0.00 | 0.00 |
| ATOM | 677 | CZ2 | TRP | 41 | −21.291 | −3.935 | 7.476 | 1.00 | 0.00 | −0.127 | 10.80 | 0.60 |
| ATOM | 678 | HZ2 | TRP | 41 | −21.776 | −3.516 | 6.594 | 1.00 | 0.00 | 0.127 | 0.00 | 0.00 |
| ATOM | 679 | CZ3 | TRP | 41 | −21.101 | −5.657 | 9.140 | 1.00 | 0.00 | −0.127 | 10.80 | 0.60 |
| ATOM | 680 | HZ3 | TRP | 41 | −21.459 | −6.602 | 9.547 | 1.00 | 0.00 | 0.127 | 0.00 | 0.00 |
| ATOM | 681 | CH2 | TRP | 41 | −21.713 | −5.126 | 8.024 | 1.00 | 0.00 | −0.127 | 10.80 | 0.60 |
| ATOM | 682 | HH2 | TRP | 41 | −22.546 | −5.659 | 7.566 | 1.00 | 0.00 | 0.127 | 0.00 | 0.00 |
| ATOM | 683 | N | THR | 42 | −17.959 | −0.942 | 12.699 | 1.00 | 0.00 | −0.650 | 9.00 | −17.40 |
| ATOM | 684 | HN | THR | 42 | −18.103 | −1.810 | 13.232 | 1.00 | 0.00 | 0.440 | 0.00 | 0.00 |
| ATOM | 685 | CA | THR | 42 | −18.637 | 0.257 | 13.109 | 1.00 | 0.00 | 0.158 | 9.40 | 4.00 |
| ATOM | 686 | HA | THR | 42 | −19.164 | 0.712 | 12.270 | 1.00 | 0.00 | 0.053 | 0.00 | 0.00 |
| ATOM | 687 | C | THR | 42 | −17.662 | 1.268 | 13.649 | 1.00 | 0.00 | 0.396 | 9.82 | 4.00 |
| ATOM | 688 | O | THR | 42 | −17.786 | 2.454 | 13.352 | 1.00 | 0.00 | −0.396 | 8.17 | −17.40 |
| ATOM | 689 | CB | THR | 42 | −19.699 | 0.005 | 14.145 | 1.00 | 0.00 | 0.060 | 9.40 | 4.00 |
| ATOM | 690 | HB | THR | 42 | −19.231 | −0.422 | 15.032 | 1.00 | 0.00 | 0.053 | 0.00 | 0.00 |
| ATOM | 691 | OG1 | THR | 42 | −20.665 | −0.906 | 13.642 | 1.00 | 0.00 | −0.537 | 11.04 | −17.40 |
| ATOM | 692 | HG1 | THR | 42 | −21.521 | −0.393 | 13.388 | 1.00 | 0.00 | 0.424 | 0.00 | 0.00 |
| ATOM | 693 | CG2 | THR | 42 | −20.377 | 1.340 | 14.496 | 1.00 | 0.00 | −0.159 | 16.15 | 4.00 |
| ATOM | 694 | HG2 | THR | 42 | −21.149 | 1.169 | 15.246 | 1.00 | 0.00 | 0.053 | 0.00 | 0.00 |
| ATOM | 695 | HG2 | THR | 42 | −19.633 | 2.032 | 14.890 | 1.00 | 0.00 | 0.053 | 0.00 | 0.00 |
| ATOM | 696 | HG2 | THR | 42 | −20.829 | 1.765 | 13.600 | 1.00 | 0.00 | 0.053 | 0.00 | 0.00 |
| ATOM | 697 | N | PRO | 43 | −16.698 | 0.861 | 14.429 | 1.00 | 0.00 | −0.422 | 9.00 | −17.40 |
| ATOM | 698 | CA | PRO | 43 | −15.743 | 1.805 | 14.950 | 1.00 | 0.00 | 0.158 | 9.40 | 4.00 |
| ATOM | 699 | HA | PRO | 43 | −16.265 | 2.747 | 15.114 | 1.00 | 0.00 | 0.053 | 0.00 | 0.00 |
| ATOM | 700 | CD | PRO | 43 | −16.956 | −0.210 | 15.382 | 1.00 | 0.00 | 0.105 | 12.77 | 4.00 |
| ATOM | 701 | HD1 | PRO | 43 | −16.474 | −1.096 | 14.968 | 1.00 | 0.00 | 0.053 | 0.00 | 0.00 |
| ATOM | 702 | HD2 | PRO | 43 | −18.040 | −0.302 | 15.436 | 1.00 | 0.00 | 0.053 | 0.00 | 0.00 |
| ATOM | 703 | C | PRO | 43 | −14.666 | 1.957 | 13.944 | 1.00 | 0.00 | 0.396 | 9.82 | 4.00 |
| ATOM | 704 | O | PRO | 43 | −14.938 | 1.780 | 12.754 | 1.00 | 0.00 | −0.396 | 8.17 | −17.40 |
| ATOM | 705 | CB | PRO | 43 | −15.224 | 1.217 | 16.262 | 1.00 | 0.00 | −0.106 | 12.77 | 4.00 |
| ATOM | 706 | HB1 | PRO | 43 | −15.102 | 2.082 | 16.913 | 1.00 | 0.00 | 0.053 | 0.00 | 0.00 |
| ATOM | 707 | HB2 | PRO | 43 | −14.288 | 0.731 | 15.985 | 1.00 | 0.00 | 0.053 | 0.00 | 0.00 |
| ATOM | 708 | CG | PRO | 43 | −16.329 | 0.248 | 16.703 | 1.00 | 0.00 | −0.106 | 12.77 | 4.00 |
| ATOM | 709 | HG1 | PRO | 43 | −17.056 | 0.750 | 17.341 | 1.00 | 0.00 | 0.053 | 0.00 | 0.00 |
| ATOM | 710 | HG2 | PRO | 43 | −15.912 | −0.589 | 17.262 | 1.00 | 0.00 | 0.053 | 0.00 | 0.00 |
| ATOM | 711 | N | ASP | 44 | −13.463 | 2.343 | 14.423 | 1.00 | 0.00 | −0.650 | 9.00 | −17.40 |
| ATOM | 712 | HN | ASP | 44 | −13.375 | 2.576 | 15.422 | 1.00 | 0.00 | 0.440 | 0.00 | 0.00 |
| ATOM | 713 | CA | ASP | 44 | −12.308 | 2.438 | 13.586 | 1.00 | 0.00 | 0.158 | 9.40 | 4.00 |
| ATOM | 714 | HA | ASP | 44 | −12.447 | 3.335 | 12.983 | 1.00 | 0.00 | 0.053 | 0.00 | 0.00 |
| ATOM | 715 | C | ASP | 44 | −12.291 | 1.201 | 12.774 | 1.00 | 0.00 | 0.396 | 9.82 | 4.00 |
| ATOM | 716 | O | ASP | 44 | −12.473 | 0.096 | 13.277 | 1.00 | 0.00 | −0.396 | 8.17 | −17.40 |
| ATOM | 717 | CB | ASP | 44 | −10.984 | 2.561 | 14.352 | 1.00 | 0.00 | −0.336 | 12.77 | 4.00 |
| ATOM | 718 | HB1 | ASP | 44 | −10.199 | 2.405 | 13.612 | 1.00 | 0.00 | 0.053 | 0.00 | 0.00 |
| ATOM | 719 | HB2 | ASP | 44 | −11.010 | 1.782 | 15.114 | 1.00 | 0.00 | 0.053 | 0.00 | 0.00 |
| ATOM | 720 | CG | ASP | 44 | −10.976 | 3.963 | 14.938 | 1.00 | 0.00 | 0.297 | 9.82 | 4.00 |
| ATOM | 721 | OD1 | ASP | 44 | −11.397 | 4.899 | 14.207 | 1.00 | 0.00 | −0.534 | 8.17 | −18.95 |
| ATOM | 722 | OD2 | ASP | 44 | −10.557 | 4.120 | 16.115 | 1.00 | 0.00 | −0.534 | 8.17 | −18.95 |
| ATOM | 723 | N | ASP | 45 | −12.100 | 1.413 | 11.470 | 1.00 | 0.00 | −0.650 | 9.00 | −17.40 |
| ATOM | 724 | HN | ASP | 45 | −11.822 | 2.364 | 11.188 | 1.00 | 0.00 | 0.440 | 0.00 | 0.00 |
| ATOM | 725 | CA | ASP | 45 | −12.243 | 0.450 | 10.430 | 1.00 | 0.00 | 0.158 | 9.40 | 4.00 |
| ATOM | 726 | HA | ASP | 45 | −11.285 | 0.037 | 10.113 | 1.00 | 0.00 | 0.053 | 0.00 | 0.00 |
| ATOM | 727 | C | ASP | 45 | −12.877 | 1.318 | 9.423 | 1.00 | 0.00 | 0.396 | 9.82 | 4.00 |
| ATOM | 728 | O | ASP | 45 | −12.678 | 1.183 | 8.216 | 1.00 | 0.00 | −0.396 | 8.17 | −17.40 |
| ATOM | 729 | CB | ASP | 45 | −13.259 | −0.668 | 10.738 | 1.00 | 0.00 | −0.336 | 12.77 | 4.00 |
| ATOM | 730 | HB1 | ASP | 45 | −14.232 | −0.204 | 10.898 | 1.00 | 0.00 | 0.053 | 0.00 | 0.00 |
| ATOM | 731 | HB2 | ASP | 45 | −12.924 | −1.190 | 11.634 | 1.00 | 0.00 | 0.053 | 0.00 | 0.00 |
| ATOM | 732 | CG | ASP | 45 | −13.298 | −1.610 | 9.546 | 1.00 | 0.00 | 0.297 | 9.82 | 4.00 |
| ATOM | 733 | OD1 | ASP | 45 | −12.211 | −2.091 | 9.132 | 1.00 | 0.00 | −0.534 | 8.17 | −18.95 |
| ATOM | 734 | OD2 | ASP | 45 | −14.417 | −1.874 | 9.034 | 1.00 | 0.00 | −0.534 | 8.17 | −18.95 |
| ATOM | 735 | N | TYR | 46 | −13.667 | 2.275 | 9.950 | 1.00 | 0.00 | −0.650 | 9.00 | −17.40 |
| ATOM | 736 | HN | TYR | 46 | −13.867 | 2.304 | 10.959 | 1.00 | 0.00 | 0.440 | 0.00 | 0.00 |
| ATOM | 737 | CA | TYR | 46 | −14.213 | 3.240 | 9.065 | 1.00 | 0.00 | 0.158 | 9.40 | 4.00 |
| ATOM | 738 | HA | TYR | 46 | −13.804 | 3.122 | 8.061 | 1.00 | 0.00 | 0.053 | 0.00 | 0.00 |
| ATOM | 739 | C | TYR | 46 | −13.814 | 4.546 | 9.673 | 1.00 | 0.00 | 0.396 | 9.82 | 4.00 |
| ATOM | 740 | O | TYR | 46 | −14.296 | 4.944 | 10.735 | 1.00 | 0.00 | −0.396 | 8.17 | −17.40 |
| ATOM | 741 | CB | TYR | 46 | −15.742 | 3.163 | 8.944 | 1.00 | 0.00 | −0.106 | 12.77 | 4.00 |
| ATOM | 742 | HB1 | TYR | 46 | −16.141 | 3.849 | 9.690 | 1.00 | 0.00 | 0.053 | 0.00 | 0.00 |
| ATOM | 743 | HB2 | TYR | 46 | −16.011 | 2.126 | 9.143 | 1.00 | 0.00 | 0.053 | 0.00 | 0.00 |
| ATOM | 744 | CG | TYR | 46 | −16.071 | 3.579 | 7.554 | 1.00 | 0.00 | 0.000 | 7.26 | 0.60 |
| ATOM | 745 | CD1 | TYR | 46 | −16.059 | 4.897 | 7.170 | 1.00 | 0.00 | −0.127 | 10.80 | 0.60 |
| ATOM | 746 | HD1 | TYR | 46 | −15.805 | 5.672 | 7.893 | 1.00 | 0.00 | 0.127 | 0.00 | 0.00 |
| ATOM | 747 | CD2 | TYR | 46 | −16.392 | 2.618 | 6.620 | 1.00 | 0.00 | −0.127 | 10.80 | 0.60 |
| ATOM | 748 | HD2 | TYR | 46 | −16.401 | 1.568 | 6.915 | 1.00 | 0.00 | 0.127 | 0.00 | 0.00 |
| ATOM | 749 | CE1 | TYR | 46 | −16.366 | 5.240 | 5.876 | 1.00 | 0.00 | −0.127 | 10.80 | 0.60 |
| ATOM | 750 | HE1 | TYR | 46 | −16.354 | 6.288 | 5.579 | 1.00 | 0.00 | 0.127 | 0.00 | 0.00 |
| ATOM | 751 | CE2 | TYR | 46 | −16.700 | 2.955 | 5.324 | 1.00 | 0.00 | −0.127 | 10.80 | 0.60 |
| ATOM | 752 | HE2 | TYR | 46 | −16.952 | 2.181 | 4.599 | 1.00 | 0.00 | 0.127 | 0.00 | 0.00 |
| ATOM | 753 | CZ | TYR | 46 | −16.687 | 4.276 | 4.951 | 1.00 | 0.00 | 0.026 | 7.26 | 0.60 |
| ATOM | 754 | OH | TYR | 46 | −17.001 | 4.641 | 3.624 | 1.00 | 0.00 | −0.451 | 10.94 | −17.40 |
| ATOM | 755 | HH | TYR | 46 | −16.826 | 5.647 | 3.493 | 1.00 | 0.00 | 0.424 | 0.00 | 0.00 |
| ATOM | 756 | N | ILE | 47 | −12.886 | 5.253 | 9.009 | 1.00 | 0.00 | −0.650 | 9.00 | −17.40 |
| ATOM | 757 | HN | ILE | 47 | −12.531 | 4.925 | 8.098 | 1.00 | 0.00 | 0.440 | 0.00 | 0.00 |
| ATOM | 758 | CA | ILE | 47 | −12.397 | 6.466 | 9.584 | 1.00 | 0.00 | 0.158 | 9.40 | 4.00 |
| ATOM | 759 | HA | ILE | 47 | −12.887 | 6.620 | 10.545 | 1.00 | 0.00 | 0.053 | 0.00 | 0.00 |
| ATOM | 760 | C | ILE | 47 | −12.708 | 7.591 | 8.662 | 1.00 | 0.00 | 0.396 | 9.82 | 4.00 |
| ATOM | 761 | O | ILE | 47 | −12.885 | 7.401 | 7.460 | 1.00 | 0.00 | −0.396 | 8.17 | −17.40 |
| ATOM | 762 | CB | ILE | 47 | −10.912 | 6.469 | 9.829 | 1.00 | 0.00 | −0.053 | 9.40 | 4.00 |
| ATOM | 763 | HB | ILE | 47 | −10.650 | 7.452 | 10.219 | 1.00 | 0.00 | 0.053 | 0.00 | 0.00 |
| ATOM | 764 | CG1 | ILE | 47 | −10.118 | 6.327 | 8.517 | 1.00 | 0.00 | −0.106 | 12.77 | 4.00 |
| ATOM | 765 | HG1 | ILE | 47 | −9.038 | 6.401 | 8.649 | 1.00 | 0.00 | 0.053 | 0.00 | 0.00 |
| ATOM | 766 | HG1 | ILE | 47 | −10.364 | 7.084 | 7.773 | 1.00 | 0.00 | 0.053 | 0.00 | 0.00 |
| ATOM | 767 | CG2 | ILE | 47 | −10.616 | 5.360 | 10.849 | 1.00 | 0.00 | −0.159 | 16.15 | 4.00 |
| ATOM | 768 | HG2 | ILE | 47 | −9.545 | 5.330 | 11.053 | 1.00 | 0.00 | 0.053 | 0.00 | 0.00 |
| ATOM | 769 | HG2 | ILE | 47 | −11.156 | 5.562 | 11.773 | 1.00 | 0.00 | 0.053 | 0.00 | 0.00 |
| ATOM | 770 | HG2 | ILE | 47 | −10.935 | 4.399 | 10.444 | 1.00 | 0.00 | 0.053 | 0.00 | 0.00 |
| ATOM | 771 | CD1 | ILE | 47 | −10.339 | 4.991 | 7.809 | 1.00 | 0.00 | −0.159 | 16.15 | 4.00 |
| ATOM | 772 | HD1 | ILE | 47 | −9.748 | 4.962 | 6.893 | 1.00 | 0.00 | 0.053 | 0.00 | 0.00 |
| ATOM | 773 | HD1 | ILE | 47 | −10.031 | 4.176 | 8.464 | 1.00 | 0.00 | 0.053 | 0.00 | 0.00 |
| ATOM | 774 | HD1 | ILE | 47 | −11.395 | 4.879 | 7.563 | 1.00 | 0.00 | 0.053 | 0.00 | 0.00 |
| ATOM | 775 | N | ASN | 48 | −12.804 | 8.805 | 9.239 | 1.00 | 0.00 | −0.650 | 9.00 | −17.40 |
| ATOM | 776 | HN | ASN | 48 | −12.679 | 8.888 | 10.258 | 1.00 | 0.00 | 0.440 | 0.00 | 0.00 |
| ATOM | 777 | CA | ASN | 48 | −13.075 | 9.980 | 8.472 | 1.00 | 0.00 | 0.158 | 9.40 | 4.00 |
| ATOM | 778 | HA | ASN | 48 | −13.485 | 9.638 | 7.521 | 1.00 | 0.00 | 0.053 | 0.00 | 0.00 |
| ATOM | 779 | C | ASN | 48 | −11.772 | 10.684 | 8.307 | 1.00 | 0.00 | 0.396 | 9.82 | 4.00 |
| ATOM | 780 | O | ASN | 48 | −11.127 | 11.079 | 9.277 | 1.00 | 0.00 | −0.396 | 8.17 | −17.40 |
| ATOM | 781 | CB | ASN | 48 | −14.050 | 10.940 | 9.170 | 1.00 | 0.00 | −0.106 | 12.77 | 4.00 |
| ATOM | 782 | HB1 | ASN | 48 | −14.082 | 11.858 | 8.584 | 1.00 | 0.00 | 0.053 | 0.00 | 0.00 |
| ATOM | 783 | HB2 | ASN | 48 | −13.668 | 11.123 | 10.174 | 1.00 | 0.00 | 0.053 | 0.00 | 0.00 |
| ATOM | 784 | CG | ASN | 48 | −15.412 | 10.261 | 9.211 | 1.00 | 0.00 | 0.396 | 9.82 | 4.00 |
| ATOM | 785 | OD1 | ASN | 48 | −16.239 | 10.449 | 8.320 | 1.00 | 0.00 | −0.396 | 8.17 | −17.40 |
| ATOM | 786 | ND2 | ASN | 48 | −15.648 | 9.436 | 10.266 | 1.00 | 0.00 | −0.879 | 13.25 | −17.40 |
| ATOM | 787 | HD2 | ASN | 48 | −14.927 | 9.307 | 10.990 | 1.00 | 0.00 | 0.440 | 0.00 | 0.00 |
| ATOM | 788 | HD2 | ASN | 48 | −16.547 | 8.940 | 10.340 | 1.00 | 0.00 | 0.440 | 0.00 | 0.00 |
| ATOM | 789 | N | MET | 49 | −11.352 | 10.849 | 7.043 | 1.00 | 0.00 | −0.650 | 9.00 | −17.40 |
| ATOM | 790 | HN | MET | 49 | −11.943 | 10.518 | 6.267 | 1.00 | 0.00 | 0.440 | 0.00 | 0.00 |
| ATOM | 791 | CA | MET | 49 | −10.101 | 11.474 | 6.753 | 1.00 | 0.00 | 0.158 | 9.40 | 4.00 |
| ATOM | 792 | HA | MET | 49 | −9.510 | 11.448 | 7.668 | 1.00 | 0.00 | 0.053 | 0.00 | 0.00 |
| ATOM | 793 | C | MET | 49 | −10.398 | 12.870 | 6.326 | 1.00 | 0.00 | 0.396 | 9.82 | 4.00 |
| ATOM | 794 | O | MET | 49 | −11.146 | 13.104 | 5.377 | 1.00 | 0.00 | −0.396 | 8.17 | −17.40 |
| ATOM | 795 | CB | MET | 49 | −9.365 | 10.793 | 5.582 | 1.00 | 0.00 | −0.106 | 12.77 | 4.00 |
| ATOM | 796 | HB1 | MET | 49 | −9.992 | 10.873 | 4.694 | 1.00 | 0.00 | 0.053 | 0.00 | 0.00 |
| ATOM | 797 | HB2 | MET | 49 | −9.200 | 9.748 | 5.846 | 1.00 | 0.00 | 0.053 | 0.00 | 0.00 |
| ATOM | 798 | CG | MET | 49 | −8.002 | 11.398 | 5.235 | 1.00 | 0.00 | −0.041 | 12.77 | 4.00 |
| ATOM | 799 | HG1 | MET | 49 | −7.337 | 11.260 | 6.088 | 1.00 | 0.00 | 0.053 | 0.00 | 0.00 |
| ATOM | 800 | HG2 | MET | 49 | −8.140 | 12.458 | 5.025 | 1.00 | 0.00 | 0.053 | 0.00 | 0.00 |
| ATOM | 801 | SD | MET | 49 | −7.203 | 10.643 | 3.781 | 1.00 | 0.00 | −0.130 | 16.39 | −6.40 |
| ATOM | 802 | CE | MET | 49 | −6.935 | 9.017 | 4.544 | 1.00 | 0.00 | −0.094 | 16.15 | 4.00 |
| ATOM | 803 | HE1 | MET | 49 | −6.443 | 8.356 | 3.829 | 1.00 | 0.00 | 0.053 | 0.00 | 0.00 |
| ATOM | 804 | HE2 | MET | 49 | −6.305 | 9.128 | 5.426 | 1.00 | 0.00 | 0.053 | 0.00 | 0.00 |
| ATOM | 805 | HE3 | MET | 49 | −7.894 | 8.588 | 4.834 | 1.00 | 0.00 | 0.053 | 0.00 | 0.00 |
| ATOM | 806 | N | THR | 50 | −9.823 | 13.846 | 7.046 | 1.00 | 0.00 | −0.650 | 9.00 | −17.40 |
| ATOM | 807 | HN | THR | 50 | −9.250 | 13.615 | 7.870 | 1.00 | 0.00 | 0.440 | 0.00 | 0.00 |
| ATOM | 808 | CA | THR | 50 | −10.013 | 15.205 | 6.659 | 1.00 | 0.00 | 0.158 | 9.40 | 4.00 |
| ATOM | 809 | HA | THR | 50 | −10.880 | 15.227 | 5.999 | 1.00 | 0.00 | 0.053 | 0.00 | 0.00 |
| ATOM | 810 | C | THR | 50 | −8.772 | 15.632 | 5.959 | 1.00 | 0.00 | 0.396 | 9.82 | 4.00 |
| ATOM | 811 | O | THR | 50 | −7.672 | 15.575 | 6.508 | 1.00 | 0.00 | −0.396 | 8.17 | −17.40 |
| ATOM | 812 | CB | THR | 50 | −10.231 | 16.117 | 7.807 | 1.00 | 0.00 | 0.060 | 9.40 | 4.00 |
| ATOM | 813 | HB | THR | 50 | −11.159 | 15.868 | 8.321 | 1.00 | 0.00 | 0.053 | 0.00 | 0.00 |
| ATOM | 814 | OG1 | THR | 50 | −10.375 | 17.435 | 7.324 | 1.00 | 0.00 | −0.537 | 11.04 | −17.40 |
| ATOM | 815 | HG1 | THR | 50 | −9.503 | 17.958 | 7.488 | 1.00 | 0.00 | 0.424 | 0.00 | 0.00 |
| ATOM | 816 | CG2 | THR | 50 | −9.076 | 16.004 | 8.811 | 1.00 | 0.00 | −0.159 | 16.15 | 4.00 |
| ATOM | 817 | HG2 | THR | 50 | −9.253 | 16.680 | 9.647 | 1.00 | 0.00 | 0.053 | 0.00 | 0.00 |
| ATOM | 818 | HG2 | THR | 50 | −9.013 | 14.980 | 9.179 | 1.00 | 0.00 | 0.053 | 0.00 | 0.00 |
| ATOM | 819 | HG2 | THR | 50 | −8.140 | 16.270 | 8.320 | 1.00 | 0.00 | 0.053 | 0.00 | 0.00 |
| ATOM | 820 | N | SER | 51 | −8.906 | 16.063 | 4.698 | 1.00 | 0.00 | −0.650 | 9.00 | −17.40 |
| ATOM | 821 | HN | SER | 51 | −9.827 | 16.123 | 4.241 | 1.00 | 0.00 | 0.440 | 0.00 | 0.00 |
| ATOM | 822 | CA | SER | 51 | −7.708 | 16.434 | 4.019 | 1.00 | 0.00 | 0.158 | 9.40 | 4.00 |
| ATOM | 823 | HA | SER | 51 | −6.883 | 16.404 | 4.731 | 1.00 | 0.00 | 0.053 | 0.00 | 0.00 |
| ATOM | 824 | C | SER | 51 | −7.878 | 17.806 | 3.480 | 1.00 | 0.00 | 0.396 | 9.82 | 4.00 |
| ATOM | 825 | O | SER | 51 | −8.950 | 18.185 | 3.010 | 1.00 | 0.00 | −0.396 | 8.17 | −17.40 |
| ATOM | 826 | CB | SER | 51 | −7.365 | 15.519 | 2.828 | 1.00 | 0.00 | 0.007 | 12.77 | 4.00 |
| ATOM | 827 | HB1 | SER | 51 | −8.170 | 15.543 | 2.093 | 1.00 | 0.00 | 0.053 | 0.00 | 0.00 |
| ATOM | 828 | HB2 | SER | 51 | −7.231 | 14.493 | 3.171 | 1.00 | 0.00 | 0.053 | 0.00 | 0.00 |
| ATOM | 829 | OG | SER | 51 | −6.164 | 15.956 | 2.208 | 1.00 | 0.00 | −0.537 | 11.04 | −17.40 |
| ATOM | 830 | HG | SER | 51 | −5.574 | 16.438 | 2.901 | 1.00 | 0.00 | 0.424 | 0.00 | 0.00 |
| ATOM | 831 | N | ASP | 52 | −6.808 | 18.611 | 3.580 | 1.00 | 0.00 | −0.650 | 9.00 | −17.40 |
| ATOM | 832 | HN | ASP | 52 | −5.959 | 18.292 | 4.070 | 1.00 | 0.00 | 0.440 | 0.00 | 0.00 |
| ATOM | 833 | CA | ASP | 52 | −6.861 | 19.914 | 2.998 | 1.00 | 0.00 | 0.158 | 9.40 | 4.00 |
| ATOM | 834 | HA | ASP | 52 | −7.917 | 20.085 | 2.790 | 1.00 | 0.00 | 0.053 | 0.00 | 0.00 |
| ATOM | 835 | C | ASP | 52 | −6.018 | 19.821 | 1.771 | 1.00 | 0.00 | 0.396 | 9.82 | 4.00 |
| ATOM | 836 | O | ASP | 52 | −5.350 | 20.775 | 1.383 | 1.00 | 0.00 | −0.396 | 8.17 | −17.40 |
| ATOM | 837 | CB | ASP | 52 | −6.252 | 21.005 | 3.895 | 1.00 | 0.00 | −0.336 | 12.77 | 4.00 |
| ATOM | 838 | HB1 | ASP | 52 | −5.549 | 20.524 | 4.575 | 1.00 | 0.00 | 0.053 | 0.00 | 0.00 |
| ATOM | 839 | HB2 | ASP | 52 | −7.065 | 21.479 | 4.443 | 1.00 | 0.00 | 0.053 | 0.00 | 0.00 |
| ATOM | 840 | CG | ASP | 52 | −5.543 | 22.007 | 2.996 | 1.00 | 0.00 | 0.297 | 9.82 | 4.00 |
| ATOM | 841 | OD1 | ASP | 52 | −4.386 | 22.384 | 3.326 | 1.00 | 0.00 | −0.534 | 8.17 | −18.95 |
| ATOM | 842 | OD2 | ASP | 52 | −6.152 | 22.409 | 1.970 | 1.00 | 0.00 | −0.534 | 8.17 | −18.95 |
| ATOM | 843 | N | CYS | 53 | −6.036 | 18.646 | 1.119 | 1.00 | 0.00 | −0.650 | 9.00 | −17.40 |
| ATOM | 844 | HN | CYS | 53 | −6.657 | 17.895 | 1.452 | 1.00 | 0.00 | 0.440 | 0.00 | 0.00 |
| ATOM | 845 | CA | CYS | 53 | −5.212 | 18.412 | −0.028 | 1.00 | 0.00 | 0.158 | 9.40 | 4.00 |
| ATOM | 846 | HA | CYS | 53 | −5.151 | 19.352 | −0.575 | 1.00 | 0.00 | 0.053 | 0.00 | 0.00 |
| ATOM | 847 | C | CYS | 53 | −5.883 | 17.333 | −0.842 | 1.00 | 0.00 | 0.396 | 9.82 | 4.00 |
| ATOM | 848 | O | CYS | 53 | −7.045 | 17.476 | −1.220 | 1.00 | 0.00 | −0.396 | 8.17 | −17.40 |
| ATOM | 849 | CB | CYS | 53 | −3.789 | 17.955 | 0.344 | 1.00 | 0.00 | −0.041 | 12.77 | 4.00 |
| ATOM | 850 | HB1 | CYS | 53 | −3.330 | 17.509 | −0.538 | 1.00 | 0.00 | 0.053 | 0.00 | 0.00 |
| ATOM | 851 | HB2 | CYS | 53 | −3.863 | 17.223 | 1.148 | 1.00 | 0.00 | 0.053 | 0.00 | 0.00 |
| ATOM | 852 | SG | CYS | 53 | −2.695 | 19.302 | 0.918 | 1.00 | 0.00 | −0.065 | 19.93 | −6.40 |
| ATOM | 853 | N | SER | 54 | −5.164 | 16.231 | −1.161 | 1.00 | 0.00 | −0.650 | 9.00 | −17.40 |
| ATOM | 854 | HN | SER | 54 | −4.193 | 16.146 | −0.827 | 1.00 | 0.00 | 0.440 | 0.00 | 0.00 |
| ATOM | 855 | CA | SER | 54 | −5.736 | 15.172 | −1.961 | 1.00 | 0.00 | 0.158 | 9.40 | 4.00 |
| ATOM | 856 | HA | SER | 54 | −6.680 | 15.513 | −2.385 | 1.00 | 0.00 | 0.053 | 0.00 | 0.00 |
| ATOM | 857 | C | SER | 54 | −5.976 | 13.966 | −1.088 | 1.00 | 0.00 | 0.396 | 9.82 | 4.00 |
| ATOM | 858 | O | SER | 54 | −5.636 | 13.977 | 0.093 | 1.00 | 0.00 | −0.396 | 8.17 | −17.40 |
| ATOM | 859 | CB | SER | 54 | −4.831 | 14.737 | −3.126 | 1.00 | 0.00 | 0.007 | 12.77 | 4.00 |
| ATOM | 860 | HB1 | SER | 54 | −5.284 | 13.898 | −3.654 | 1.00 | 0.00 | 0.053 | 0.00 | 0.00 |
| ATOM | 861 | HB2 | SER | 54 | −3.856 | 14.432 | −2.744 | 1.00 | 0.00 | 0.053 | 0.00 | 0.00 |
| ATOM | 862 | OG | SER | 54 | −4.657 | 15.817 | −4.032 | 1.00 | 0.00 | −0.537 | 11.04 | −17.40 |
| ATOM | 863 | HG | SER | 54 | −3.876 | 16.409 | −3.714 | 1.00 | 0.00 | 0.424 | 0.00 | 0.00 |
| ATOM | 864 | N | SER | 55 | −6.583 | 12.884 | −1.644 | 1.00 | 0.00 | −0.650 | 9.00 | −17.40 |
| ATOM | 865 | HN | SER | 55 | −6.824 | 12.883 | −2.645 | 1.00 | 0.00 | 0.440 | 0.00 | 0.00 |
| ATOM | 866 | CA | SER | 55 | −6.887 | 11.735 | −0.824 | 1.00 | 0.00 | 0.158 | 9.40 | 4.00 |
| ATOM | 867 | HA | SER | 55 | −6.188 | 11.647 | 0.008 | 1.00 | 0.00 | 0.053 | 0.00 | 0.00 |
| ATOM | 868 | C | SER | 55 | −6.809 | 10.462 | −1.620 | 1.00 | 0.00 | 0.396 | 9.82 | 4.00 |
| ATOM | 869 | O | SER | 55 | −6.373 | 10.455 | −2.770 | 1.00 | 0.00 | −0.396 | 8.17 | −17.40 |
| ATOM | 870 | CB | SER | 55 | −8.302 | 11.777 | −0.225 | 1.00 | 0.00 | 0.007 | 12.77 | 4.00 |
| ATOM | 871 | HB1 | SER | 55 | −8.506 | 10.854 | 0.318 | 1.00 | 0.00 | 0.053 | 0.00 | 0.00 |
| ATOM | 872 | HB2 | SER | 55 | −9.041 | 11.889 | −1.017 | 1.00 | 0.00 | 0.053 | 0.00 | 0.00 |
| ATOM | 873 | OG | SER | 55 | −8.421 | 12.872 | 0.670 | 1.00 | 0.00 | −0.537 | 11.04 | −17.40 |
| ATOM | 874 | HG | SER | 55 | −7.700 | 13.574 | 0.451 | 1.00 | 0.00 | 0.424 | 0.00 | 0.00 |
| ATOM | 875 | N | PHE | 56 | −7.228 | 9.333 | −0.993 | 1.00 | 0.00 | −0.650 | 9.00 | −17.40 |
| ATOM | 876 | HN | PHE | 56 | −7.584 | 9.399 | −0.029 | 1.00 | 0.00 | 0.440 | 0.00 | 0.00 |
| ATOM | 877 | CA | PHE | 56 | −7.188 | 8.046 | −1.636 | 1.00 | 0.00 | 0.158 | 9.40 | 4.00 |
| ATOM | 878 | HA | PHE | 56 | −6.712 | 8.116 | −2.614 | 1.00 | 0.00 | 0.053 | 0.00 | 0.00 |
| ATOM | 879 | C | PHE | 56 | −8.573 | 7.503 | −1.829 | 1.00 | 0.00 | 0.396 | 9.82 | 4.00 |
| ATOM | 880 | O | PHE | 56 | −9.565 | 8.223 | −1.758 | 1.00 | 0.00 | −0.396 | 8.17 | −17.40 |
| ATOM | 881 | CB | PHE | 56 | −6.338 | 6.998 | −0.899 | 1.00 | 0.00 | −0.106 | 12.77 | 4.00 |
| ATOM | 882 | HB1 | PHE | 56 | −6.530 | 6.050 | −1.402 | 1.00 | 0.00 | 0.053 | 0.00 | 0.00 |
| ATOM | 883 | HB2 | PHE | 56 | −6.685 | 7.005 | 0.134 | 1.00 | 0.00 | 0.053 | 0.00 | 0.00 |
| ATOM | 884 | CG | PHE | 56 | −4.923 | 7.450 | −1.037 | 1.00 | 0.00 | 0.000 | 7.26 | 0.60 |
| ATOM | 885 | CD1 | PHE | 56 | −4.227 | 7.239 | −2.206 | 1.00 | 0.00 | −0.127 | 10.80 | 0.60 |
| ATOM | 886 | HD1 | PHE | 56 | −4.714 | 6.734 | −3.040 | 1.00 | 0.00 | 0.127 | 0.00 | 0.00 |
| ATOM | 887 | CD2 | PHE | 56 | −4.292 | 8.087 | 0.005 | 1.00 | 0.00 | −0.127 | 10.80 | 0.60 |
| ATOM | 888 | HD2 | PHE | 56 | −4.830 | 8.260 | 0.936 | 1.00 | 0.00 | 0.127 | 0.00 | 0.00 |
| ATOM | 889 | CE1 | PHE | 56 | −2.923 | 7.658 | −2.332 | 1.00 | 0.00 | −0.127 | 10.80 | 0.60 |
| ATOM | 890 | HE1 | PHE | 56 | −2.382 | 7.484 | −3.262 | 1.00 | 0.00 | 0.127 | 0.00 | 0.00 |
| ATOM | 891 | CE2 | PHE | 56 | −2.988 | 8.508 | −0.115 | 1.00 | 0.00 | −0.127 | 10.80 | 0.60 |
| ATOM | 892 | HE2 | PHE | 56 | −2.499 | 9.011 | 0.719 | 1.00 | 0.00 | 0.127 | 0.00 | 0.00 |
| ATOM | 893 | CZ | PHE | 56 | −2.301 | 8.295 | −1.285 | 1.00 | 0.00 | −0.127 | 10.80 | 0.60 |
| ATOM | 894 | HZ | PHE | 56 | −1.268 | 8.629 | −1.382 | 1.00 | 0.00 | 0.127 | 0.00 | 0.00 |
| ATOM | 895 | N | ILE | 57 | −8.664 | 6.180 | −2.056 | 1.00 | 0.00 | −0.650 | 9.00 | −17.40 |
| ATOM | 896 | HN | ILE | 57 | −7.829 | 5.591 | −1.925 | 1.00 | 0.00 | 0.440 | 0.00 | 0.00 |
| ATOM | 897 | CA | ILE | 57 | −9.890 | 5.566 | −2.475 | 1.00 | 0.00 | 0.158 | 9.40 | 4.00 |
| ATOM | 898 | HA | ILE | 57 | −10.456 | 6.180 | −3.174 | 1.00 | 0.00 | 0.053 | 0.00 | 0.00 |
| ATOM | 899 | C | ILE | 57 | −10.831 | 5.280 | −1.326 | 1.00 | 0.00 | 0.396 | 9.82 | 4.00 |
| ATOM | 900 | O | ILE | 57 | −10.960 | 6.063 | −0.387 | 1.00 | 0.00 | −0.396 | 8.17 | −17.40 |
| ATOM | 901 | CB | ILE | 57 | −9.612 | 4.330 | −3.299 | 1.00 | 0.00 | −0.053 | 9.40 | 4.00 |
| ATOM | 902 | HB | ILE | 57 | −8.907 | 4.591 | −4.088 | 1.00 | 0.00 | 0.053 | 0.00 | 0.00 |
| ATOM | 903 | CG1 | ILE | 57 | −10.831 | 3.891 | −4.130 | 1.00 | 0.00 | −0.106 | 12.77 | 4.00 |
| ATOM | 904 | HG1 | ILE | 57 | −11.288 | 4.709 | −4.685 | 1.00 | 0.00 | 0.053 | 0.00 | 0.00 |
| ATOM | 905 | HG1 | ILE | 57 | −11.631 | 3.465 | −3.525 | 1.00 | 0.00 | 0.053 | 0.00 | 0.00 |
| ATOM | 906 | CG2 | ILE | 57 | −9.012 | 3.255 | −2.380 | 1.00 | 0.00 | −0.159 | 16.15 | 4.00 |
| ATOM | 907 | HG2 | ILE | 57 | −8.805 | 2.355 | −2.959 | 1.00 | 0.00 | 0.053 | 0.00 | 0.00 |
| ATOM | 908 | HG2 | ILE | 57 | −8.085 | 3.626 | −1.942 | 1.00 | 0.00 | 0.053 | 0.00 | 0.00 |
| ATOM | 909 | HG2 | ILE | 57 | −9.719 | 3.019 | −1.584 | 1.00 | 0.00 | 0.053 | 0.00 | 0.00 |
| ATOM | 910 | CD1 | ILE | 57 | −10.496 | 2.826 | −5.173 | 1.00 | 0.00 | −0.159 | 16.15 | 4.00 |
| ATOM | 911 | HD1 | ILE | 57 | −11.397 | 2.560 | −5.724 | 1.00 | 0.00 | 0.053 | 0.00 | 0.00 |
| ATOM | 912 | HD1 | ILE | 57 | −9.749 | 3.216 | −5.864 | 1.00 | 0.00 | 0.053 | 0.00 | 0.00 |
| ATOM | 913 | HD1 | ILE | 57 | −10.101 | 1.940 | −4.675 | 1.00 | 0.00 | 0.053 | 0.00 | 0.00 |
| ATOM | 914 | N | LYS | 58 | −11.518 | 4.121 | −1.417 | 1.00 | 0.00 | −0.650 | 9.00 | −17.40 |
| ATOM | 915 | HN | LYS | 58 | −11.208 | 3.493 | −2.173 | 1.00 | 0.00 | 0.440 | 0.00 | 0.00 |
| ATOM | 916 | CA | LYS | 58 | −12.613 | 3.620 | −0.629 | 1.00 | 0.00 | 0.158 | 9.40 | 4.00 |
| ATOM | 917 | HA | LYS | 58 | −13.415 | 4.357 | −0.653 | 1.00 | 0.00 | 0.053 | 0.00 | 0.00 |
| ATOM | 918 | C | LYS | 58 | −12.282 | 3.361 | 0.808 | 1.00 | 0.00 | 0.396 | 9.82 | 4.00 |
| ATOM | 919 | O | LYS | 58 | −13.188 | 3.370 | 1.639 | 1.00 | 0.00 | −0.396 | 8.17 | −17.40 |
| ATOM | 920 | CB | LYS | 58 | −13.175 | 2.303 | −1.193 | 1.00 | 0.00 | −0.106 | 12.77 | 4.00 |
| ATOM | 921 | HB1 | LYS | 58 | −13.886 | 1.897 | −0.473 | 1.00 | 0.00 | 0.053 | 0.00 | 0.00 |
| ATOM | 922 | HB2 | LYS | 58 | −12.345 | 1.612 | −1.342 | 1.00 | 0.00 | 0.053 | 0.00 | 0.00 |
| ATOM | 923 | CG | LYS | 58 | −13.901 | 2.456 | −2.531 | 1.00 | 0.00 | −0.106 | 12.77 | 4.00 |
| ATOM | 924 | HG1 | LYS | 58 | −14.257 | 1.510 | −2.938 | 1.00 | 0.00 | 0.053 | 0.00 | 0.00 |
| ATOM | 925 | HG2 | LYS | 58 | −13.278 | 2.891 | −3.312 | 1.00 | 0.00 | 0.053 | 0.00 | 0.00 |
| ATOM | 926 | CD | LYS | 58 | −15.136 | 3.354 | −2.450 | 1.00 | 0.00 | −0.106 | 12.77 | 4.00 |
| ATOM | 927 | HD1 | LYS | 58 | −15.697 | 3.399 | −3.383 | 1.00 | 0.00 | 0.053 | 0.00 | 0.00 |
| ATOM | 928 | HD2 | LYS | 58 | −14.900 | 4.389 | −2.202 | 1.00 | 0.00 | 0.053 | 0.00 | 0.00 |
| ATOM | 929 | CE | LYS | 58 | −16.144 | 2.902 | −1.391 | 1.00 | 0.00 | 0.099 | 12.77 | 4.00 |
| ATOM | 930 | HE1 | LYS | 58 | −17.002 | 3.573 | −1.382 | 1.00 | 0.00 | 0.053 | 0.00 | 0.00 |
| ATOM | 931 | HE2 | LYS | 58 | −15.678 | 2.910 | −0.405 | 1.00 | 0.00 | 0.053 | 0.00 | 0.00 |
| ATOM | 932 | NZ | LYS | 58 | −16.611 | 1.530 | −1.689 | 1.00 | 0.00 | −0.045 | 13.25 | −39.20 |
| ATOM | 933 | HZ1 | LYS | 58 | −17.288 | 1.234 | −0.971 | 1.00 | 0.00 | 0.280 | 0.00 | 0.00 |
| ATOM | 934 | HZ2 | LYS | 58 | −17.062 | 1.513 | −2.614 | 1.00 | 0.00 | 0.280 | 0.00 | 0.00 |
| ATOM | 935 | HZ3 | LYS | 58 | −15.807 | 0.885 | −1.688 | 1.00 | 0.00 | 0.280 | 0.00 | 0.00 |
| ATOM | 936 | N | ARG | 59 | −11.004 | 3.125 | 1.154 | 1.00 | 0.00 | −0.650 | 9.00 | −17.40 |
| ATOM | 937 | HN | ARG | 59 | −10.255 | 3.240 | 0.456 | 1.00 | 0.00 | 0.440 | 0.00 | 0.00 |
| ATOM | 938 | CA | ARG | 59 | −10.675 | 2.712 | 2.495 | 1.00 | 0.00 | 0.158 | 9.40 | 4.00 |
| ATOM | 939 | HA | ARG | 59 | −11.098 | 1.731 | 2.714 | 1.00 | 0.00 | 0.053 | 0.00 | 0.00 |
| ATOM | 940 | C | ARG | 59 | −11.212 | 3.685 | 3.500 | 1.00 | 0.00 | 0.396 | 9.82 | 4.00 |
| ATOM | 941 | O | ARG | 59 | −11.654 | 3.281 | 4.576 | 1.00 | 0.00 | −0.396 | 8.17 | −17.40 |
| ATOM | 942 | CB | ARG | 59 | −9.160 | 2.570 | 2.721 | 1.00 | 0.00 | −0.106 | 12.77 | 4.00 |
| ATOM | 943 | HB1 | ARG | 59 | −8.975 | 2.536 | 3.794 | 1.00 | 0.00 | 0.053 | 0.00 | 0.00 |
| ATOM | 944 | HB2 | ARG | 59 | −8.664 | 3.431 | 2.273 | 1.00 | 0.00 | 0.053 | 0.00 | 0.00 |
| ATOM | 945 | CG | ARG | 59 | −8.547 | 1.307 | 2.103 | 1.00 | 0.00 | −0.106 | 12.77 | 4.00 |
| ATOM | 946 | HG1 | ARG | 59 | −9.117 | 0.445 | 2.451 | 1.00 | 0.00 | 0.053 | 0.00 | 0.00 |
| ATOM | 947 | HG2 | ARG | 59 | −7.508 | 1.238 | 2.427 | 1.00 | 0.00 | 0.053 | 0.00 | 0.00 |
| ATOM | 948 | CD | ARG | 59 | −8.555 | 1.277 | 0.572 | 1.00 | 0.00 | 0.374 | 12.77 | 4.00 |
| ATOM | 949 | HD1 | ARG | 59 | −7.976 | 2.136 | 0.232 | 1.00 | 0.00 | 0.053 | 0.00 | 0.00 |
| ATOM | 950 | HD2 | ARG | 59 | −9.596 | 1.340 | 0.256 | 1.00 | 0.00 | 0.053 | 0.00 | 0.00 |
| ATOM | 951 | NE | ARG | 59 | −7.928 | −0.012 | 0.156 | 1.00 | 0.00 | −0.819 | 9.00 | −24.67 |
| ATOM | 952 | HE | ARG | 59 | −7.539 | −0.644 | 0.870 | 1.00 | 0.00 | 0.407 | 0.00 | 0.00 |
| ATOM | 953 | CZ | ARG | 59 | −7.864 | −0.361 | −1.162 | 1.00 | 0.00 | 0.796 | 6.95 | 4.00 |
| ATOM | 954 | NH1 | ARG | 59 | −8.377 | 0.473 | −2.113 | 1.00 | 0.00 | −0.746 | 9.00 | −24.67 |
| ATOM | 955 | HH1 | ARG | 59 | −8.329 | 0.210 | −3.107 | 1.00 | 0.00 | 0.407 | 0.00 | 0.00 |
| ATOM | 956 | HH1 | ARG | 59 | −8.811 | 1.364 | −1.835 | 1.00 | 0.00 | 0.407 | 0.00 | 0.00 |
| ATOM | 957 | NH2 | ARG | 59 | −7.292 | −1.545 | −1.530 | 1.00 | 0.00 | −0.746 | 9.00 | −24.67 |
| ATOM | 958 | HH2 | ARG | 59 | −7.244 | −1.806 | −2.525 | 1.00 | 0.00 | 0.407 | 0.00 | 0.00 |
| ATOM | 959 | HH2 | ARG | 59 | −6.907 | −2.176 | −0.812 | 1.00 | 0.00 | 0.407 | 0.00 | 0.00 |
| ATOM | 960 | N | ARG | 60 | −11.199 | 4.995 | 3.197 | 1.00 | 0.00 | −0.650 | 9.00 | −17.40 |
| ATOM | 961 | HN | ARG | 60 | −10.835 | 5.335 | 2.295 | 1.00 | 0.00 | 0.440 | 0.00 | 0.00 |
| ATOM | 962 | CA | ARG | 60 | −11.714 | 5.895 | 4.186 | 1.00 | 0.00 | 0.158 | 9.40 | 4.00 |
| ATOM | 963 | HA | ARG | 60 | −12.191 | 5.303 | 4.967 | 1.00 | 0.00 | 0.053 | 0.00 | 0.00 |
| ATOM | 964 | C | ARG | 60 | −12.707 | 6.798 | 3.523 | 1.00 | 0.00 | 0.396 | 9.82 | 4.00 |
| ATOM | 965 | O | ARG | 60 | −12.864 | 6.778 | 2.302 | 1.00 | 0.00 | −0.396 | 8.17 | −17.40 |
| ATOM | 966 | CB | ARG | 60 | −10.622 | 6.803 | 4.776 | 1.00 | 0.00 | −0.106 | 12.77 | 4.00 |
| ATOM | 967 | HB1 | ARG | 60 | −9.827 | 6.169 | 5.171 | 1.00 | 0.00 | 0.053 | 0.00 | 0.00 |
| ATOM | 968 | HB2 | ARG | 60 | −11.067 | 7.401 | 5.570 | 1.00 | 0.00 | 0.053 | 0.00 | 0.00 |
| ATOM | 969 | CG | ARG | 60 | −9.987 | 7.770 | 3.765 | 1.00 | 0.00 | −0.106 | 12.77 | 4.00 |
| ATOM | 970 | HG1 | ARG | 60 | −9.120 | 8.230 | 4.239 | 1.00 | 0.00 | 0.053 | 0.00 | 0.00 |
| ATOM | 971 | HG2 | ARG | 60 | −10.732 | 8.520 | 3.503 | 1.00 | 0.00 | 0.053 | 0.00 | 0.00 |
| ATOM | 972 | CD | ARG | 60 | −9.504 | 7.137 | 2.457 | 1.00 | 0.00 | 0.374 | 12.77 | 4.00 |
| ATOM | 973 | HD1 | ARG | 60 | −9.171 | 7.935 | 1.794 | 1.00 | 0.00 | 0.053 | 0.00 | 0.00 |
| ATOM | 974 | HD2 | ARG | 60 | −10.337 | 6.591 | 2.014 | 1.00 | 0.00 | 0.053 | 0.00 | 0.00 |
| ATOM | 975 | NE | ARG | 60 | −8.378 | 6.213 | 2.774 | 1.00 | 0.00 | −0.819 | 9.00 | −24.67 |
| ATOM | 976 | HE | ARG | 60 | −7.939 | 6.229 | 3.705 | 1.00 | 0.00 | 0.407 | 0.00 | 0.00 |
| ATOM | 977 | CZ | ARG | 60 | −7.933 | 5.345 | 1.819 | 1.00 | 0.00 | 0.796 | 6.95 | 4.00 |
| ATOM | 978 | NH1 | ARG | 60 | −8.506 | 5.345 | 0.579 | 1.00 | 0.00 | −0.746 | 9.00 | −24.67 |
| ATOM | 979 | HH1 | ARG | 60 | −8.171 | 4.690 | −0.142 | 1.00 | 0.00 | 0.407 | 0.00 | 0.00 |
| ATOM | 980 | HH1 | ARG | 60 | −9.272 | 5.999 | 0.365 | 1.00 | 0.00 | 0.407 | 0.00 | 0.00 |
| ATOM | 981 | NH2 | ARG | 60 | −6.915 | 4.479 | 2.093 | 1.00 | 0.00 | −0.746 | 9.00 | −24.67 |
| ATOM | 982 | HH2 | ARG | 60 | −6.584 | 3.826 | 1.367 | 1.00 | 0.00 | 0.407 | 0.00 | 0.00 |
| ATOM | 983 | HH2 | ARG | 60 | −6.477 | 4.477 | 3.025 | 1.00 | 0.00 | 0.407 | 0.00 | 0.00 |
| ATOM | 984 | N | LYS | 61 | −13.433 | 7.592 | 4.343 | 1.00 | 0.00 | −0.650 | 9.00 | −17.40 |
| ATOM | 985 | HN | LYS | 61 | −13.331 | 7.493 | 5.363 | 1.00 | 0.00 | 0.440 | 0.00 | 0.00 |
| ATOM | 986 | CA | LYS | 61 | −14.343 | 8.570 | 3.816 | 1.00 | 0.00 | 0.158 | 9.40 | 4.00 |
| ATOM | 987 | HA | LYS | 61 | −14.585 | 8.247 | 2.803 | 1.00 | 0.00 | 0.053 | 0.00 | 0.00 |
| ATOM | 988 | C | LYS | 61 | −13.617 | 9.872 | 3.841 | 1.00 | 0.00 | 0.396 | 9.82 | 4.00 |
| ATOM | 989 | O | LYS | 61 | −12.994 | 10.235 | 4.839 | 1.00 | 0.00 | −0.396 | 8.17 | −17.40 |
| ATOM | 990 | CB | LYS | 61 | −15.654 | 8.741 | 4.604 | 1.00 | 0.00 | −0.106 | 12.77 | 4.00 |
| ATOM | 991 | HB1 | LYS | 61 | −16.070 | 9.720 | 4.370 | 1.00 | 0.00 | 0.053 | 0.00 | 0.00 |
| ATOM | 992 | HB2 | LYS | 61 | −15.428 | 8.665 | 5.667 | 1.00 | 0.00 | 0.053 | 0.00 | 0.00 |
| ATOM | 993 | CG | LYS | 61 | −16.716 | 7.690 | 4.275 | 1.00 | 0.00 | −0.106 | 12.77 | 4.00 |
| ATOM | 994 | HG1 | LYS | 61 | −16.330 | 6.670 | 4.275 | 1.00 | 0.00 | 0.053 | 0.00 | 0.00 |
| ATOM | 995 | HG2 | LYS | 61 | −17.168 | 7.825 | 3.292 | 1.00 | 0.00 | 0.053 | 0.00 | 0.00 |
| ATOM | 996 | CD | LYS | 61 | −17.886 | 7.680 | 5.261 | 1.00 | 0.00 | −0.106 | 12.77 | 4.00 |
| ATOM | 997 | HD1 | LYS | 61 | −17.494 | 7.471 | 6.256 | 1.00 | 0.00 | 0.053 | 0.00 | 0.00 |
| ATOM | 998 | HD2 | LYS | 61 | −18.586 | 6.903 | 4.954 | 1.00 | 0.00 | 0.053 | 0.00 | 0.00 |
| ATOM | 999 | CE | LYS | 61 | −18.655 | 9.000 | 5.330 | 1.00 | 0.00 | 0.099 | 12.77 | 4.00 |
| ATOM | 1000 | HE1 | LYS | 61 | −19.084 | 9.221 | 4.352 | 1.00 | 0.00 | 0.053 | 0.00 | 0.00 |
| ATOM | 1001 | HE2 | LYS | 61 | −17.975 | 9.801 | 5.619 | 1.00 | 0.00 | 0.053 | 0.00 | 0.00 |
| ATOM | 1002 | NZ | LYS | 61 | −19.739 | 8.887 | 6.331 | 1.00 | 0.00 | −0.045 | 13.25 | −39.20 |
| ATOM | 1003 | HZ1 | LYS | 61 | −20.257 | 9.775 | 6.378 | 1.00 | 0.00 | 0.280 | 0.00 | 0.00 |
| ATOM | 1004 | HZ2 | LYS | 61 | −20.381 | 8.129 | 6.058 | 1.00 | 0.00 | 0.280 | 0.00 | 0.00 |
| ATOM | 1005 | HZ3 | LYS | 61 | −19.333 | 8.677 | 7.254 | 1.00 | 0.00 | 0.280 | 0.00 | 0.00 |
| ATOM | 1006 | N | TYR | 62 | −13.677 | 10.619 | 2.724 | 1.00 | 0.00 | −0.650 | 9.00 | −17.40 |
| ATOM | 1007 | HN | TYR | 62 | −14.278 | 10.343 | 1.934 | 1.00 | 0.00 | 0.440 | 0.00 | 0.00 |
| ATOM | 1008 | CA | TYR | 62 | −12.883 | 11.805 | 2.665 | 1.00 | 0.00 | 0.158 | 9.40 | 4.00 |
| ATOM | 1009 | HA | TYR | 62 | −12.185 | 11.869 | 3.499 | 1.00 | 0.00 | 0.053 | 0.00 | 0.00 |
| ATOM | 1010 | C | TYR | 62 | −13.720 | 13.038 | 2.703 | 1.00 | 0.00 | 0.396 | 9.82 | 4.00 |
| ATOM | 1011 | O | TYR | 62 | −14.777 | 13.131 | 2.080 | 1.00 | 0.00 | −0.396 | 8.17 | −17.40 |
| ATOM | 1012 | CB | TYR | 62 | −11.965 | 11.877 | 1.426 | 1.00 | 0.00 | −0.106 | 12.77 | 4.00 |
| ATOM | 1013 | HB1 | TYR | 62 | −11.222 | 11.080 | 1.472 | 1.00 | 0.00 | 0.053 | 0.00 | 0.00 |
| ATOM | 1014 | HB2 | TYR | 62 | −11.457 | 12.841 | 1.401 | 1.00 | 0.00 | 0.053 | 0.00 | 0.00 |
| ATOM | 1015 | CG | TYR | 62 | −12.781 | 11.718 | 0.186 | 1.00 | 0.00 | 0.000 | 7.26 | 0.60 |
| ATOM | 1016 | CD1 | TYR | 62 | −13.508 | 12.767 | −0.329 | 1.00 | 0.00 | −0.127 | 10.80 | 0.60 |
| ATOM | 1017 | HD1 | TYR | 62 | −13.495 | 13.732 | 0.177 | 1.00 | 0.00 | 0.127 | 0.00 | 0.00 |
| ATOM | 1018 | CD2 | TYR | 62 | −12.805 | 10.509 | −0.473 | 1.00 | 0.00 | −0.127 | 10.80 | 0.60 |
| ATOM | 1019 | HD2 | TYR | 62 | −12.229 | 9.671 | −0.079 | 1.00 | 0.00 | 0.127 | 0.00 | 0.00 |
| ATOM | 1020 | CE1 | TYR | 62 | −14.250 | 12.609 | −1.477 | 1.00 | 0.00 | −0.127 | 10.80 | 0.60 |
| ATOM | 1021 | HE1 | TYR | 62 | −14.822 | 13.447 | −1.873 | 1.00 | 0.00 | 0.127 | 0.00 | 0.00 |
| ATOM | 1022 | CE2 | TYR | 62 | −13.545 | 10.344 | −1.621 | 1.00 | 0.00 | −0.127 | 10.80 | 0.60 |
| ATOM | 1023 | HE2 | TYR | 62 | −13.555 | 9.380 | −2.129 | 1.00 | 0.00 | 0.127 | 0.00 | 0.00 |
| ATOM | 1024 | CZ | TYR | 62 | −14.271 | 11.396 | −2.125 | 1.00 | 0.00 | 0.026 | 7.26 | 0.60 |
| ATOM | 1025 | OH | TYR | 62 | −15.031 | 11.230 | −3.302 | 1.00 | 0.00 | −0.451 | 10.94 | −17.40 |
| ATOM | 1026 | HH | TYR | 62 | −15.330 | 12.151 | −3.650 | 1.00 | 0.00 | 0.424 | 0.00 | 0.00 |
| ATOM | 1027 | N | ILE | 63 | −13.236 | 14.018 | 3.491 | 1.00 | 0.00 | −0.650 | 9.00 | −17.40 |
| ATOM | 1028 | HN | ILE | 63 | −12.386 | 13.823 | 4.039 | 1.00 | 0.00 | 0.440 | 0.00 | 0.00 |
| ATOM | 1029 | CA | ILE | 63 | −13.832 | 15.312 | 3.611 | 1.00 | 0.00 | 0.158 | 9.40 | 4.00 |
| ATOM | 1030 | HA | ILE | 63 | −14.689 | 15.409 | 2.944 | 1.00 | 0.00 | 0.053 | 0.00 | 0.00 |
| ATOM | 1031 | C | ILE | 63 | −12.728 | 16.247 | 3.231 | 1.00 | 0.00 | 0.396 | 9.82 | 4.00 |
| ATOM | 1032 | O | ILE | 63 | −11.572 | 16.007 | 3.579 | 1.00 | 0.00 | −0.396 | 8.17 | −17.40 |
| ATOM | 1033 | CB | ILE | 63 | −14.222 | 15.652 | 5.018 | 1.00 | 0.00 | −0.053 | 9.40 | 4.00 |
| ATOM | 1034 | HB | ILE | 63 | −13.322 | 15.580 | 5.630 | 1.00 | 0.00 | 0.053 | 0.00 | 0.00 |
| ATOM | 1035 | CG1 | ILE | 63 | −15.213 | 14.619 | 5.577 | 1.00 | 0.00 | −0.106 | 12.77 | 4.00 |
| ATOM | 1036 | HG1 | ILE | 63 | −15.496 | 14.806 | 6.612 | 1.00 | 0.00 | 0.053 | 0.00 | 0.00 |
| ATOM | 1037 | HG1 | ILE | 63 | −14.828 | 13.599 | 5.565 | 1.00 | 0.00 | 0.053 | 0.00 | 0.00 |
| ATOM | 1038 | CG2 | ILE | 63 | −14.781 | 17.079 | 5.004 | 1.00 | 0.00 | −0.159 | 16.15 | 4.00 |
| ATOM | 1039 | HG2 | ILE | 63 | −15.076 | 17.363 | 6.013 | 1.00 | 0.00 | 0.053 | 0.00 | 0.00 |
| ATOM | 1040 | HG2 | ILE | 63 | −14.015 | 17.766 | 4.642 | 1.00 | 0.00 | 0.053 | 0.00 | 0.00 |
| ATOM | 1041 | HG2 | ILE | 63 | −15.648 | 17.123 | 4.345 | 1.00 | 0.00 | 0.053 | 0.00 | 0.00 |
| ATOM | 1042 | CD1 | ILE | 63 | −16.529 | 14.560 | 4.805 | 1.00 | 0.00 | −0.159 | 16.15 | 4.00 |
| ATOM | 1043 | HD1 | ILE | 63 | −17.180 | 13.810 | 5.253 | 1.00 | 0.00 | 0.053 | 0.00 | 0.00 |
| ATOM | 1044 | HD1 | ILE | 63 | −17.016 | 15.534 | 4.842 | 1.00 | 0.00 | 0.053 | 0.00 | 0.00 |
| ATOM | 1045 | HD1 | ILE | 63 | −16.330 | 14.293 | 3.767 | 1.00 | 0.00 | 0.053 | 0.00 | 0.00 |
| ATOM | 1046 | N | VAL | 64 | −13.034 | 17.332 | 2.495 | 1.00 | 0.00 | −0.650 | 9.00 | −17.40 |
| ATOM | 1047 | HN | VAL | 64 | −14.007 | 17.545 | 2.235 | 1.00 | 0.00 | 0.440 | 0.00 | 0.00 |
| ATOM | 1048 | CA | VAL | 64 | −11.952 | 18.179 | 2.086 | 1.00 | 0.00 | 0.158 | 9.40 | 4.00 |
| ATOM | 1049 | HA | VAL | 64 | −11.008 | 17.773 | 2.451 | 1.00 | 0.00 | 0.053 | 0.00 | 0.00 |
| ATOM | 1050 | C | VAL | 64 | −12.146 | 19.550 | 2.644 | 1.00 | 0.00 | 0.396 | 9.82 | 4.00 |
| ATOM | 1051 | O | VAL | 64 | −13.269 | 20.029 | 2.798 | 1.00 | 0.00 | −0.396 | 8.17 | −17.40 |
| ATOM | 1052 | CB | VAL | 64 | −11.826 | 18.314 | 0.596 | 1.00 | 0.00 | −0.053 | 9.40 | 4.00 |
| ATOM | 1053 | HB | VAL | 64 | −11.023 | 19.019 | 0.380 | 1.00 | 0.00 | 0.053 | 0.00 | 0.00 |
| ATOM | 1054 | CG1 | VAL | 64 | −11.499 | 16.931 | 0.008 | 1.00 | 0.00 | −0.159 | 16.15 | 4.00 |
| ATOM | 1055 | HG1 | VAL | 64 | −11.403 | 17.009 | −1.075 | 1.00 | 0.00 | 0.053 | 0.00 | 0.00 |
| ATOM | 1056 | HG1 | VAL | 64 | −10.561 | 16.568 | 0.429 | 1.00 | 0.00 | 0.053 | 0.00 | 0.00 |
| ATOM | 1057 | HG1 | VAL | 64 | −12.300 | 16.233 | 0.252 | 1.00 | 0.00 | 0.053 | 0.00 | 0.00 |
| ATOM | 1058 | CG2 | VAL | 64 | −13.120 | 18.938 | 0.049 | 1.00 | 0.00 | −0.159 | 16.15 | 4.00 |
| ATOM | 1059 | HG2 | VAL | 64 | −13.044 | 19.042 | −1.033 | 1.00 | 0.00 | 0.053 | 0.00 | 0.00 |
| ATOM | 1060 | HG2 | VAL | 64 | −13.965 | 18.294 | 0.292 | 1.00 | 0.00 | 0.053 | 0.00 | 0.00 |
| ATOM | 1061 | HG2 | VAL | 64 | −13.269 | 19.919 | 0.499 | 1.00 | 0.00 | 0.053 | 0.00 | 0.00 |
| ATOM | 1062 | N | GLU | 65 | −11.016 | 20.206 | 2.975 | 1.00 | 0.00 | −0.650 | 9.00 | −17.40 |
| ATOM | 1063 | HN | GLU | 65 | −10.112 | 19.729 | 2.842 | 1.00 | 0.00 | 0.440 | 0.00 | 0.00 |
| ATOM | 1064 | CA | GLU | 65 | −11.019 | 21.538 | 3.503 | 1.00 | 0.00 | 0.158 | 9.40 | 4.00 |
| ATOM | 1065 | HA | GLU | 65 | −11.931 | 21.664 | 4.086 | 1.00 | 0.00 | 0.053 | 0.00 | 0.00 |
| ATOM | 1066 | C | GLU | 65 | −10.984 | 22.486 | 2.353 | 1.00 | 0.00 | 0.396 | 9.82 | 4.00 |
| ATOM | 1067 | O | GLU | 65 | −10.748 | 22.096 | 1.212 | 1.00 | 0.00 | −0.396 | 8.17 | −17.40 |
| ATOM | 1068 | CB | GLU | 65 | −9.793 | 21.828 | 4.384 | 1.00 | 0.00 | −0.106 | 12.77 | 4.00 |
| ATOM | 1069 | HB1 | GLU | 65 | −8.898 | 21.725 | 3.769 | 1.00 | 0.00 | 0.053 | 0.00 | 0.00 |
| ATOM | 1070 | HB2 | GLU | 65 | −9.781 | 21.107 | 5.202 | 1.00 | 0.00 | 0.053 | 0.00 | 0.00 |
| ATOM | 1071 | CG | GLU | 65 | −9.776 | 23.228 | 4.998 | 1.00 | 0.00 | −0.106 | 12.77 | 4.00 |
| ATOM | 1072 | HG1 | GLU | 65 | −10.651 | 23.321 | 5.641 | 1.00 | 0.00 | 0.053 | 0.00 | 0.00 |
| ATOM | 1073 | HG2 | GLU | 65 | −9.810 | 23.950 | 4.182 | 1.00 | 0.00 | 0.053 | 0.00 | 0.00 |
| ATOM | 1074 | CD | GLU | 65 | −8.491 | 23.370 | 5.800 | 1.00 | 0.00 | 0.399 | 9.82 | 4.00 |
| ATOM | 1075 | OE1 | GLU | 65 | −7.676 | 22.410 | 5.781 | 1.00 | 0.00 | −0.396 | 8.17 | −18.95 |
| ATOM | 1076 | OE2 | GLU | 65 | −8.304 | 24.440 | 6.437 | 1.00 | 0.00 | −0.427 | 8.17 | −18.95 |
| ATOM | 1078 | N | PRO | 66 | −11.243 | 23.734 | 2.637 | 1.00 | 0.00 | −0.422 | 9.00 | −17.40 |
| ATOM | 1079 | CA | PRO | 66 | −11.189 | 24.702 | 1.576 | 1.00 | 0.00 | 0.158 | 9.40 | 4.00 |
| ATOM | 1080 | HA | PRO | 66 | −11.647 | 24.300 | 0.671 | 1.00 | 0.00 | 0.053 | 0.00 | 0.00 |
| ATOM | 1081 | CD | PRO | 66 | −12.320 | 24.032 | 3.570 | 1.00 | 0.00 | 0.105 | 12.77 | 4.00 |
| ATOM | 1082 | HD1 | PRO | 66 | −11.837 | 24.256 | 4.521 | 1.00 | 0.00 | 0.053 | 0.00 | 0.00 |
| ATOM | 1083 | HD2 | PRO | 66 | −12.936 | 23.134 | 3.611 | 1.00 | 0.00 | 0.053 | 0.00 | 0.00 |
| ATOM | 1084 | C | PRO | 66 | −9.769 | 25.062 | 1.270 | 1.00 | 0.00 | 0.396 | 9.82 | 4.00 |
| ATOM | 1085 | O | PRO | 66 | −8.961 | 25.170 | 2.191 | 1.00 | 0.00 | −0.396 | 8.17 | −17.40 |
| ATOM | 1086 | CB | PRO | 66 | −12.047 | 25.880 | 2.022 | 1.00 | 0.00 | −0.106 | 12.77 | 4.00 |
| ATOM | 1087 | HB1 | PRO | 66 | −12.536 | 26.351 | 1.169 | 1.00 | 0.00 | 0.053 | 0.00 | 0.00 |
| ATOM | 1088 | HB2 | PRO | 66 | −11.442 | 26.636 | 2.521 | 1.00 | 0.00 | 0.053 | 0.00 | 0.00 |
| ATOM | 1089 | CG | PRO | 66 | −13.065 | 25.239 | 2.981 | 1.00 | 0.00 | −0.106 | 12.77 | 4.00 |
| ATOM | 1090 | HG1 | PRO | 66 | −13.906 | 24.975 | 2.340 | 1.00 | 0.00 | 0.053 | 0.00 | 0.00 |
| ATOM | 1091 | HG2 | PRO | 66 | −13.283 | 26.025 | 3.703 | 1.00 | 0.00 | 0.053 | 0.00 | 0.00 |
| ATOM | 1092 | N | LEU | 67 | −9.459 | 25.240 | −0.029 | 1.00 | 0.00 | −0.650 | 9.00 | −17.40 |
| ATOM | 1093 | HN | LEU | 67 | −10.223 | 25.176 | −0.715 | 1.00 | 0.00 | 0.440 | 0.00 | 0.00 |
| ATOM | 1094 | CA | LEU | 67 | −8.139 | 25.512 | −0.525 | 1.00 | 0.00 | 0.158 | 9.40 | 4.00 |
| ATOM | 1095 | HA | LEU | 67 | −7.399 | 24.809 | −0.140 | 1.00 | 0.00 | 0.053 | 0.00 | 0.00 |
| ATOM | 1096 | C | LEU | 67 | −7.662 | 26.880 | −0.149 | 1.00 | 0.00 | 0.396 | 9.82 | 4.00 |
| ATOM | 1097 | O | LEU | 67 | −6.472 | 27.052 | 0.107 | 1.00 | 0.00 | −0.396 | 8.17 | −17.40 |
| ATOM | 1098 | CB | LEU | 67 | −8.049 | 25.393 | −2.055 | 1.00 | 0.00 | −0.106 | 12.77 | 4.00 |
| ATOM | 1099 | HB1 | LEU | 67 | −7.075 | 25.687 | −2.448 | 1.00 | 0.00 | 0.053 | 0.00 | 0.00 |
| ATOM | 1100 | HB2 | LEU | 67 | −8.778 | 26.015 | −2.573 | 1.00 | 0.00 | 0.053 | 0.00 | 0.00 |
| ATOM | 1101 | CG | LEU | 67 | −8.288 | 23.959 | −2.561 | 1.00 | 0.00 | −0.053 | 9.40 | 4.00 |
| ATOM | 1102 | HG | LEU | 67 | −8.283 | 23.921 | −3.650 | 1.00 | 0.00 | 0.053 | 0.00 | 0.00 |
| ATOM | 1103 | CD1 | LEU | 67 | −7.195 | 23.004 | −2.053 | 1.00 | 0.00 | −0.159 | 16.15 | 4.00 |
| ATOM | 1104 | HD1 | LEU | 67 | −7.388 | 21.998 | −2.425 | 1.00 | 0.00 | 0.053 | 0.00 | 0.00 |
| ATOM | 1105 | HD1 | LEU | 67 | −6.222 | 23.341 | −2.409 | 1.00 | 0.00 | 0.053 | 0.00 | 0.00 |
| ATOM | 1106 | HD1 | LEU | 67 | −7.198 | 22.994 | −0.963 | 1.00 | 0.00 | 0.053 | 0.00 | 0.00 |
| ATOM | 1107 | CD2 | LEU | 67 | −9.708 | 23.474 | −2.228 | 1.00 | 0.00 | −0.159 | 16.15 | 4.00 |
| ATOM | 1108 | HD2 | LEU | 67 | −9.842 | 22.458 | −2.599 | 1.00 | 0.00 | 0.053 | 0.00 | 0.00 |
| ATOM | 1109 | HD2 | LEU | 67 | −9.853 | 23.487 | −1.147 | 1.00 | 0.00 | 0.053 | 0.00 | 0.00 |
| ATOM | 1110 | HD2 | LEU | 67 | −10.437 | 24.132 | −2.700 | 1.00 | 0.00 | 0.053 | 0.00 | 0.00 |
| ATOM | 1111 | N | SER | 68 | −8.576 | 27.874 | −0.108 | 1.00 | 0.00 | −0.650 | 9.00 | −17.40 |
| ATOM | 1112 | HN | SER | 68 | −9.567 | 27.623 | −0.231 | 1.00 | 0.00 | 0.440 | 0.00 | 0.00 |
| ATOM | 1113 | CA | SER | 68 | −8.252 | 29.263 | 0.098 | 1.00 | 0.00 | 0.158 | 9.40 | 4.00 |
| ATOM | 1114 | HA | SER | 68 | −7.828 | 29.701 | −0.805 | 1.00 | 0.00 | 0.053 | 0.00 | 0.00 |
| ATOM | 1115 | C | SER | 68 | −7.253 | 29.420 | 1.201 | 1.00 | 0.00 | 0.396 | 9.82 | 4.00 |
| ATOM | 1116 | O | SER | 68 | −7.358 | 28.802 | 2.260 | 1.00 | 0.00 | −0.396 | 8.17 | −17.40 |
| ATOM | 1117 | CB | SER | 68 | −9.473 | 30.133 | 0.439 | 1.00 | 0.00 | 0.007 | 12.77 | 4.00 |
| ATOM | 1118 | HB1 | SER | 68 | −9.948 | 29.772 | 1.351 | 1.00 | 0.00 | 0.053 | 0.00 | 0.00 |
| ATOM | 1119 | HB2 | SER | 68 | −10.199 | 30.096 | −0.373 | 1.00 | 0.00 | 0.053 | 0.00 | 0.00 |
| ATOM | 1120 | OG | SER | 68 | −9.074 | 31.481 | 0.634 | 1.00 | 0.00 | −0.537 | 11.04 | −17.40 |
| ATOM | 1121 | HG | SER | 68 | −8.055 | 31.560 | 0.505 | 1.00 | 0.00 | 0.424 | 0.00 | 0.00 |
| ATOM | 1122 | N | LYS | 69 | −6.228 | 30.257 | 0.941 | 1.00 | 0.00 | −0.650 | 9.00 | −17.40 |
| ATOM | 1123 | HN | LYS | 69 | −6.201 | 30.749 | 0.036 | 1.00 | 0.00 | 0.440 | 0.00 | 0.00 |
| ATOM | 1124 | CA | LYS | 69 | −5.176 | 30.480 | 1.886 | 1.00 | 0.00 | 0.158 | 9.40 | 4.00 |
| ATOM | 1125 | HA | LYS | 69 | −5.097 | 29.601 | 2.525 | 1.00 | 0.00 | 0.053 | 0.00 | 0.00 |
| ATOM | 1126 | C | LYS | 69 | −5.518 | 31.687 | 2.694 | 1.00 | 0.00 | 0.396 | 9.82 | 4.00 |
| ATOM | 1127 | O | LYS | 69 | −6.478 | 32.398 | 2.402 | 1.00 | 0.00 | −0.396 | 8.17 | −17.40 |
| ATOM | 1128 | CB | LYS | 69 | −3.808 | 30.733 | 1.231 | 1.00 | 0.00 | −0.106 | 12.77 | 4.00 |
| ATOM | 1129 | HB1 | LYS | 69 | −3.028 | 30.986 | 1.949 | 1.00 | 0.00 | 0.053 | 0.00 | 0.00 |
| ATOM | 1130 | HB2 | LYS | 69 | −3.820 | 31.553 | 0.513 | 1.00 | 0.00 | 0.053 | 0.00 | 0.00 |
| ATOM | 1131 | CG | LYS | 69 | −3.276 | 29.522 | 0.459 | 1.00 | 0.00 | −0.106 | 12.77 | 4.00 |
| ATOM | 1132 | HG1 | LYS | 69 | −4.016 | 29.056 | −0.191 | 1.00 | 0.00 | 0.053 | 0.00 | 0.00 |
| ATOM | 1133 | HG2 | LYS | 69 | −2.919 | 28.715 | 1.099 | 1.00 | 0.00 | 0.053 | 0.00 | 0.00 |
| ATOM | 1134 | CD | LYS | 69 | −2.095 | 29.847 | −0.458 | 1.00 | 0.00 | −0.106 | 12.77 | 4.00 |
| ATOM | 1135 | HD1 | LYS | 69 | −2.204 | 30.786 | −0.998 | 1.00. | 0.00 | 0.053 | 0.00 | 0.00 |
| ATOM | 1136 | HD2 | LYS | 69 | −1.920 | 29.098 | −1.230 | 1.00 | 0.00 | 0.053 | 0.00 | 0.00 |
| ATOM | 1137 | CE | LYS | 69 | −0.760 | 29.971 | 0.277 | 1.00 | 0.00 | 0.099 | 12.77 | 4.00 |
| ATOM | 1138 | HE1 | LYS | 69 | −0.525 | 29.035 | 0.784 | 1.00 | 0.00 | 0.053 | 0.00 | 0.00 |
| ATOM | 1139 | HE2 | LYS | 69 | −0.814 | 30.770 | 1.015 | 1.00 | 0.00 | 0.053 | 0.00 | 0.00 |
| ATOM | 1140 | NZ | LYS | 69 | 0.321 | 30.279 | −0.686 | 1.00 | 0.00 | −0.045 | 13.25 | −39.20 |
| ATOM | 1141 | HZ1 | LYS | 69 | 1.216 | 30.360 | −0.183 | 1.00 | 0.00 | 0.280 | 0.00 | 0.00 |
| ATOM | 1142 | HZ2 | LYS | 69 | 0.386 | 29.523 | −1.382 | 1.00 | 0.00 | 0.280 | 0.00 | 0.00 |
| ATOM | 1143 | HZ3 | LYS | 69 | 0.113 | 31.167 | −1.163 | 1.00 | 0.00 | 0.280 | 0.00 | 0.00 |
| ATOM | 1144 | N | GLU | 70 | −4.733 | 31.932 | 3.762 | 1.00 | 0.00 | −0.650 | 9.00 | −17.40 |
| ATOM | 1145 | HN | GLU | 70 | −3.954 | 31.294 | 3.979 | 1.00 | 0.00 | 0.440 | 0.00 | 0.00 |
| ATOM | 1146 | CA | GLU | 70 | −4.971 | 33.070 | 4.598 | 1.00 | 0.00 | 0.158 | 9.40 | 4.00 |
| ATOM | 1147 | HA | GLU | 70 | −5.817 | 33.613 | 4.177 | 1.00 | 0.00 | 0.053 | 0.00 | 0.00 |
| ATOM | 1148 | C | GLU | 70 | −3.732 | 33.900 | 4.588 | 1.00 | 0.00 | 0.396 | 9.82 | 4.00 |
| ATOM | 1149 | O | GLU | 70 | −2.638 | 33.404 | 4.321 | 1.00 | 0.00 | −0.396 | 8.17 | −17.40 |
| ATOM | 1150 | CB | GLU | 70 | −5.266 | 32.713 | 6.066 | 1.00 | 0.00 | −0.106 | 12.77 | 4.00 |
| ATOM | 1151 | HB1 | GLU | 70 | −6.139 | 32.073 | 6.191 | 1.00 | 0.00 | 0.053 | 0.00 | 0.00 |
| ATOM | 1152 | HB2 | GLU | 70 | −5.457 | 33.582 | 6.695 | 1.00 | 0.00 | 0.053 | 0.00 | 0.00 |
| ATOM | 1153 | CG | GLU | 70 | −4.123 | 31.966 | 6.754 | 1.00 | 0.00 | −0.106 | 12.77 | 4.00 |
| ATOM | 1154 | HG1 | GLU | 70 | −4.308 | 31.988 | 7.828 | 1.00 | 0.00 | 0.053 | 0.00 | 0.00 |
| ATOM | 1155 | HG2 | GLU | 70 | −3.192 | 32.477 | 6.506 | 1.00 | 0.00 | 0.053 | 0.00 | 0.00 |
| ATOM | 1156 | CD | GLU | 70 | −4.117 | 30.538 | 6.230 | 1.00 | 0.00 | 0.399 | 9.82 | 4.00 |
| ATOM | 1157 | OE1 | GLU | 70 | −5.140 | 29.831 | 6.431 | 1.00 | 0.00 | −0.396 | 8.17 | −18.95 |
| ATOM | 1158 | OE2 | GLU | 70 | −3.091 | 30.135 | 5.618 | 1.00 | 0.00 | −0.427 | 8.17 | −18.95 |
| ATOM | 1160 | N | GLU | 71 | −3.878 | 35.210 | 4.863 | 1.00 | 0.00 | −0.650 | 9.00 | −17.40 |
| ATOM | 1161 | HN | GLU | 71 | −4.811 | 35.600 | 5.054 | 1.00 | 0.00 | 0.440 | 0.00 | 0.00 |
| ATOM | 1162 | CA | GLU | 71 | −2.722 | 36.055 | 4.887 | 1.00 | 0.00 | 0.158 | 9.40 | 4.00 |
| ATOM | 1163 | HA | GLU | 71 | −1.977 | 35.571 | 4.254 | 1.00 | 0.00 | 0.053 | 0.00 | 0.00 |
| ATOM | 1164 | C | GLU | 71 | −2.294 | 36.135 | 6.312 | 1.00 | 0.00 | 0.396 | 9.82 | 4.00 |
| ATOM | 1165 | O | GLU | 71 | −3.110 | 36.353 | 7.206 | 1.00 | 0.00 | −0.396 | 8.17 | −17.40 |
| ATOM | 1166 | CB | GLU | 71 | −2.984 | 37.485 | 4.377 | 1.00 | 0.00 | −0.106 | 12.77 | 4.00 |
| ATOM | 1167 | HB1 | GLU | 71 | −3.385 | 37.418 | 3.365 | 1.00 | 0.00 | 0.053 | 0.00 | 0.00 |
| ATOM | 1168 | HB2 | GLU | 71 | −2.038 | 38.027 | 4.380 | 1.00 | 0.00 | 0.053 | 0.00 | 0.00 |
| ATOM | 1169 | CG | GLU | 71 | −3.981 | 38.285 | 5.220 | 1.00 | 0.00 | −0.106 | 12.77 | 4.00 |
| ATOM | 1170 | HG1 | GLU | 71 | −4.723 | 37.592 | 5.616 | 1.00 | 0.00 | 0.053 | 0.00 | 0.00 |
| ATOM | 1171 | HG2 | GLU | 71 | −4.450 | 39.028 | 4.575 | 1.00 | 0.00 | 0.053 | 0.00 | 0.00 |
| ATOM | 1172 | CD | GLU | 71 | −3.214 | 38.958 | 6.350 | 1.00 | 0.00 | 0.399 | 9.82 | 4.00 |
| ATOM | 1173 | OE1 | GLU | 71 | −1.958 | 38.857 | 6.356 | 1.00 | 0.00 | −0.396 | 8.17 | −18.95 |
| ATOM | 1174 | OE2 | GLU | 71 | −3.876 | 39.580 | 7.224 | 1.00 | 0.00 | −0.427 | 8.17 | −18.95 |
| ATOM | 1176 | N | ALA | 72 | −0.988 | 35.929 | 6.564 | 1.00 | 0.00 | −0.650 | 9.00 | −17.40 |
| ATOM | 1177 | HN | ALA | 72 | −0.329 | 35.755 | 5.790 | 1.00 | 0.00 | 0.440 | 0.00 | 0.00 |
| ATOM | 1178 | CA | ALA | 72 | −0.523 | 35.954 | 7.918 | 1.00 | 0.00 | 0.158 | 9.40 | 4.00 |
| ATOM | 1179 | HA | ALA | 72 | −1.295 | 36.408 | 8.538 | 1.00 | 0.00 | 0.053 | 0.00 | 0.00 |
| ATOM | 1180 | C | ALA | 72 | 0.731 | 36.760 | 7.970 | 1.00 | 0.00 | 0.396 | 9.82 | 4.00 |
| ATOM | 1181 | O | ALA | 72 | 1.153 | 37.351 | 6.977 | 1.00 | 0.00 | −0.396 | 8.17 | −17.40 |
| ATOM | 1182 | CB | ALA | 72 | −0.185 | 34.561 | 8.477 | 1.00 | 0.00 | −0.159 | 16.15 | 4.00 |
| ATOM | 1183 | HB1 | ALA | 72 | 0.160 | 34.656 | 9.506 | 1.00 | 0.00 | 0.053 | 0.00 | 0.00 |
| ATOM | 1184 | HB2 | ALA | 72 | −1.074 | 33.932 | 8.449 | 1.00 | 0.00 | 0.053 | 0.00 | 0.00 |
| ATOM | 1185 | HB3 | ALA | 72 | 0.599 | 34.106 | 7.871 | 1.00 | 0.00 | 0.053 | 0.00 | 0.00 |
| ATOM | 1186 | N | GLU | 73 | 1.337 | 36.823 | 9.172 | 1.00 | 0.00 | −0.650 | 9.00 | −17.40 |
| ATOM | 1187 | HN | GLU | 73 | 0.916 | 36.331 | 9.974 | 1.00 | 0.00 | 0.440 | 0.00 | 0.00 |
| ATOM | 1188 | CA | GLU | 73 | 2.551 | 37.557 | 9.359 | 1.00 | 0.00 | 0.158 | 9.40 | 4.00 |
| ATOM | 1189 | HA | GLU | 73 | 2.410 | 38.556 | 8.946 | 1.00 | 0.00 | 0.053 | 0.00 | 0.00 |
| ATOM | 1190 | C | GLU | 73 | 3.646 | 36.837 | 8.641 | 1.00 | 0.00 | 0.396 | 9.82 | 4.00 |
| ATOM | 1191 | O | GLU | 73 | 4.257 | 37.383 | 7.724 | 1.00 | 0.00 | −0.396 | 8.17 | −17.40 |
| ATOM | 1192 | CB | GLU | 73 | 2.965 | 37.660 | 10.836 | 1.00 | 0.00 | −0.106 | 12.77 | 4.00 |
| ATOM | 1193 | HB1 | GLU | 73 | 3.937 | 38.128 | 10.985 | 1.00 | 0.00 | 0.053 | 0.00 | 0.00 |
| ATOM | 1194 | HB2 | GLU | 73 | 3.036 | 36.695 | 11.338 | 1.00 | 0.00 | 0.053 | 0.00 | 0.00 |
| ATOM | 1195 | CG | GLU | 73 | 1.995 | 38.480 | 11.689 | 1.00 | 0.00 | −0.106 | 12.77 | 4.00 |
| ATOM | 1196 | HG1 | GLU | 73 | 2.292 | 38.377 | 12.732 | 1.00 | 0.00 | 0.053 | 0.00 | 0.00 |
| ATOM | 1197 | HG2 | GLU | 73 | 0.990 | 38.087 | 11.531 | 1.00 | 0.00 | 0.053 | 0.00 | 0.00 |
| ATOM | 1198 | CD | GLU | 73 | 2.084 | 39.932 | 11.242 | 1.00 | 0.00 | 0.399 | 9.82 | 4.00 |
| ATOM | 1199 | OE1 | GLU | 73 | 3.228 | 40.448 | 11.128 | 1.00 | 0.00 | −0.396 | 8.17 | −18.95 |
| ATOM | 1200 | OE2 | GLU | 73 | 1.008 | 40.543 | 11.005 | 1.00 | 0.00 | −0.427 | 8.17 | −18.95 |
| ATOM | 1202 | N | PHE | 74 | 3.911 | 35.571 | 9.027 | 1.00 | 0.00 | −0.650 | 9.00 | −17.40 |
| ATOM | 1203 | HN | PHE | 74 | 3.353 | 35.123 | 9.768 | 1.00 | 0.00 | 0.440 | 0.00 | 0.00 |
| ATOM | 1204 | CA | PHE | 74 | 4.980 | 34.859 | 8.389 | 1.00 | 0.00 | 0.158 | 9.40 | 4.00 |
| ATOM | 1205 | HA | PHE | 74 | 5.153 | 35.274 | 7.396 | 1.00 | 0.00 | 0.053 | 0.00 | 0.00 |
| ATOM | 1206 | C | PHE | 74 | 4.613 | 33.403 | 8.265 | 1.00 | 0.00 | 0.396 | 9.82 | 4.00 |
| ATOM | 1207 | O | PHE | 74 | 4.104 | 33.015 | 7.215 | 1.00 | 0.00 | −0.396 | 8.17 | −17.40 |
| ATOM | 1208 | CB | PHE | 74 | 6.341 | 34.989 | 9.108 | 1.00 | 0.00 | −0.106 | 12.77 | 4.00 |
| ATOM | 1209 | HB1 | PHE | 74 | 7.048 | 34.314 | 8.625 | 1.00 | 0.00 | 0.053 | 0.00 | 0.00 |
| ATOM | 1210 | HB2 | PHE | 74 | 6.202 | 34.716 | 10.154 | 1.00 | 0.00 | 0.053 | 0.00 | 0.00 |
| ATOM | 1211 | CG | PHE | 74 | 6.800 | 36.404 | 8.990 | 1.00 | 0.00 | 0.000 | 7.26 | 0.60 |
| ATOM | 1212 | CD1 | PHE | 74 | 7.451 | 36.835 | 7.857 | 1.00 | 0.00 | −0.127 | 10.80 | 0.60 |
| ATOM | 1213 | HD1 | PHE | 74 | 7.630 | 36.136 | 7.039 | 1.00 | 0.00 | 0.127 | 0.00 | 0.00 |
| ATOM | 1214 | CD2 | PHE | 74 | 6.587 | 37.297 | 10.015 | 1.00 | 0.00 | −0.127 | 10.80 | 0.60 |
| ATOM | 1215 | HD2 | PHE | 74 | 6.076 | 36.967 | 10.920 | 1.00 | 0.00 | 0.127 | 0.00 | 0.00 |
| ATOM | 1216 | CE1 | PHE | 74 | 7.878 | 38.137 | 7.745 | 1.00 | 0.00 | −0.127 | 10.80 | 0.60 |
| ATOM | 1217 | HE1 | PHE | 74 | 8.391 | 38.466 | 6.841 | 1.00 | 0.00 | 0.127 | 0.00 | 0.00 |
| ATOM | 1218 | CE2 | PHE | 74 | 7.012 | 38.601 | 9.909 | 1.00 | 0.00 | −0.127 | 10.80 | 0.60 |
| ATOM | 1219 | HE2 | PHE | 74 | 6.836 | 39.299 | 10.727 | 1.00 | 0.00 | 0.127 | 0.00 | 0.00 |
| ATOM | 1220 | CZ | PHE | 74 | 7.658 | 39.023 | 8.772 | 1.00 | 0.00 | −0.127 | 10.80 | 0.60 |
| ATOM | 1221 | HZ | PHE | 74 | 7.994 | 40.056 | 8.685 | 1.00 | 0.00 | 0.127 | 0.00 | 0.00 |
| ATOM | 1222 | N | PRO | 75 | 4.854 | 32.583 | 9.275 | 1.00 | 0.00 | −0.422 | 9.00 | −17.40 |
| ATOM | 1223 | CA | PRO | 75 | 4.639 | 31.156 | 9.167 | 1.00 | 0.00 | 0.158 | 9.40 | 4.00 |
| ATOM | 1224 | HA | PRO | 75 | 5.366 | 30.674 | 8.513 | 1.00 | 0.00 | 0.053 | 0.00 | 0.00 |
| ATOM | 1225 | CD | PRO | 75 | 4.661 | 33.025 | 10.652 | 1.00 | 0.00 | 0.105 | 12.77 | 4.00 |
| ATOM | 1226 | HD1 | PRO | 75 | 3.865 | 33.769 | 10.617 | 1.00 | 0.00 | 0.053 | 0.00 | 0.00 |
| ATOM | 1227 | HD2 | PRO | 75 | 5.617 | 33.442 | 10.968 | 1.00 | 0.00 | 0.053 | 0.00 | 0.00 |
| ATOM | 1228 | C | PRO | 75 | 3.289 | 30.822 | 8.614 | 1.00 | 0.00 | 0.396 | 9.82 | 4.00 |
| ATOM | 1229 | O | PRO | 75 | 2.309 | 30.874 | 9.355 | 1.00 | 0.00 | −0.396 | 8.17 | −17.40 |
| ATOM | 1230 | CB | PRO | 75 | 4.785 | 30.606 | 10.581 | 1.00 | 0.00 | −0.106 | 12.77 | 4.00 |
| ATOM | 1231 | HB1 | PRO | 75 | 5.823 | 30.359 | 10.803 | 1.00 | 0.00 | 0.053 | 0.00 | 0.00 |
| ATOM | 1232 | HB2 | PRO | 75 | 4.194 | 29.699 | 10.715 | 1.00 | 0.00 | 0.053 | 0.00 | 0.00 |
| ATOM | 1233 | CG | PRO | 75 | 4.267 | 31.768 | 11.446 | 1.00 | 0.00 | −0.106 | 12.77 | 4.00 |
| ATOM | 1234 | HG1 | PRO | 75 | 4.731 | 31.755 | 12.432 | 1.00 | 0.00 | 0.053 | 0.00 | 0.00 |
| ATOM | 1235 | HG2 | PRO | 75 | 3.187 | 31.701 | 11.580 | 1.00 | 0.00 | 0.053 | 0.00 | 0.00 |
| ATOM | 1236 | N | ILE | 76 | 3.229 | 30.458 | 7.320 | 1.00 | 0.00 | −0.650 | 9.00 | −17.40 |
| ATOM | 1237 | HN | ILE | 76 | 4.102 | 30.416 | 6.775 | 1.00 | 0.00 | 0.440 | 0.00 | 0.00 |
| ATOM | 1238 | CA | ILE | 76 | 1.996 | 30.126 | 6.672 | 1.00 | 0.00 | 0.158 | 9.40 | 4.00 |
| ATOM | 1239 | HA | ILE | 76 | 1.241 | 30.875 | 6.911 | 1.00 | 0.00 | 0.053 | 0.00 | 0.00 |
| ATOM | 1240 | C | ILE | 76 | 1.525 | 28.787 | 7.140 | 1.00 | 0.00 | 0.396 | 9.82 | 4.00 |
| ATOM | 1241 | O | ILE | 76 | 0.331 | 28.573 | 7.345 | 1.00 | 0.00 | −0.396 | 8.17 | −17.40 |
| ATOM | 1242 | CB | ILE | 76 | 2.130 | 30.078 | 5.176 | 1.00 | 0.00 | −0.053 | 9.40 | 4.00 |
| ATOM | 1243 | HB | ILE | 76 | 2.517 | 31.038 | 4.835 | 1.00 | 0.00 | 0.053 | 0.00 | 0.00 |
| ATOM | 1244 | CG1 | ILE | 76 | 0.747 | 29.957 | 4.515 | 1.00 | 0.00 | −0.106 | 12.77 | 4.00 |
| ATOM | 1245 | HG1 | ILE | 76 | 0.155 | 29.123 | 4.894 | 1.00 | 0.00 | 0.053 | 0.00 | 0.00 |
| ATOM | 1246 | HG1 | ILE | 76 | 0.793 | 29.806 | 3.436 | 1.00 | 0.00 | 0.053 | 0.00 | 0.00 |
| ATOM | 1247 | CG2 | ILE | 76 | 3.103 | 28.943 | 4.814 | 1.00 | 0.00 | −0.159 | 16.15 | 4.00 |
| ATOM | 1248 | HG2 | ILE | 76 | 3.214 | 28.890 | 3.731 | 1.00 | 0.00 | 0.053 | 0.00 | 0.00 |
| ATOM | 1249 | HG2 | ILE | 76 | 4.074 | 29.136 | 5.269 | 1.00 | 0.00 | 0.053 | 0.00 | 0.00 |
| ATOM | 1250 | HG2 | ILE | 76 | 2.711 | 27.995 | 5.184 | 1.00 | 0.00 | 0.053 | 0.00 | 0.00 |
| ATOM | 1251 | CD1 | ILE | 76 | −0.131 | 31.193 | 4.710 | 1.00 | 0.00 | −0.159 | 16.15 | 4.00 |
| ATOM | 1252 | HD1 | ILE | 76 | −1.091 | 31.038 | 4.218 | 1.00 | 0.00 | 0.053 | 0.00 | 0.00 |
| ATOM | 1253 | HD1 | ILE | 76 | −0.291 | 31.360 | 5.774 | 1.00 | 0.00 | 0.053 | 0.00 | 0.00 |
| ATOM | 1254 | HD1 | ILE | 76 | 0.363 | 32.062 | 4.276 | 1.00 | 0.00 | 0.053 | 0.00 | 0.00 |
| ATOM | 1255 | N | ALA | 77 | 2.470 | 27.853 | 7.343 | 1.00 | 0.00 | −0.650 | 9.00 | −17.40 |
| ATOM | 1256 | HN | ALA | 77 | 3.461 | 28.117 | 7.255 | 1.00 | 0.00 | 0.440 | 0.00 | 0.00 |
| ATOM | 1257 | CA | ALA | 77 | 2.139 | 26.500 | 7.678 | 1.00 | 0.00 | 0.158 | 9.40 | 4.00 |
| ATOM | 1258 | HA | ALA | 77 | 1.508 | 26.066 | 6.901 | 1.00 | 0.00 | 0.053 | 0.00 | 0.00 |
| ATOM | 1259 | C | ALA | 77 | 1.402 | 26.460 | 8.979 | 1.00 | 0.00 | 0.396 | 9.82 | 4.00 |
| ATOM | 1260 | O | ALA | 77 | 0.434 | 25.717 | 9.130 | 1.00 | 0.00 | −0.396 | 8.17 | −17.40 |
| ATOM | 1261 | CB | ALA | 77 | 3.385 | 25.613 | 7.805 | 1.00 | 0.00 | −0.159 | 16.15 | 4.00 |
| ATOM | 1262 | HB1 | ALA | 77 | 3.083 | 24.596 | 8.059 | 1.00 | 0.00 | 0.053 | 0.00 | 0.00 |
| ATOM | 1263 | HB2 | ALA | 77 | 3.923 | 25.604 | 6.857 | 1.00 | 0.00 | 0.053 | 0.00 | 0.00 |
| ATOM | 1264 | HB3 | ALA | 77 | 4.033 | 26.006 | 8.587 | 1.00 | 0.00 | 0.053 | 0.00 | 0.00 |
| ATOM | 1265 | N | TYR | 78 | 1.829 | 27.262 | 9.966 | 1.00 | 0.00 | −0.650 | 9.00 | −17.40 |
| ATOM | 1266 | HN | TYR | 78 | 2.617 | 27.906 | 9.812 | 1.00 | 0.00 | 0.440 | 0.00 | 0.00 |
| ATOM | 1267 | CA | TYR | 78 | 1.170 | 27.207 | 11.237 | 1.00 | 0.00 | 0.158 | 9.40 | 4.00 |
| ATOM | 1268 | HA | TYR | 78 | 1.201 | 26.186 | 11.617 | 1.00 | 0.00 | 0.053 | 0.00 | 0.00 |
| ATOM | 1269 | C | TYR | 78 | −0.255 | 27.638 | 11.078 | 1.00 | 0.00 | 0.396 | 9.82 | 4.00 |
| ATOM | 1270 | O | TYR | 78 | −1.158 | 27.067 | 11.688 | 1.00 | 0.00 | −0.396 | 8.17 | −17.40 |
| ATOM | 1271 | CB | TYR | 78 | 1.819 | 28.120 | 12.289 | 1.00 | 0.00 | −0.106 | 12.77 | 4.00 |
| ATOM | 1272 | HB1 | TYR | 78 | 1.184 | 28.143 | 13.175 | 1.00 | 0.00 | 0.053 | 0.00 | 0.00 |
| ATOM | 1273 | HB2 | TYR | 78 | 1.917 | 29.122 | 11.872 | 1.00 | 0.00 | 0.053 | 0.00 | 0.00 |
| ATOM | 1274 | CG | TYR | 78 | 3.163 | 27.576 | 12.636 | 1.00 | 0.00 | 0.000 | 7.26 | 0.60 |
| ATOM | 1275 | CD1 | TYR | 78 | 4.266 | 27.877 | 11.870 | 1.00 | 0.00 | −0.127 | 10.80 | 0.60 |
| ATOM | 1276 | HD1 | TYR | 78 | 4.157 | 28.519 | 10.996 | 1.00 | 0.00 | 0.127 | 0.00 | 0.00 |
| ATOM | 1277 | CD2 | TYR | 78 | 3.317 | 26.762 | 13.735 | 1.00 | 0.00 | −0.127 | 10.80 | 0.60 |
| ATOM | 1278 | HD2 | TYR | 78 | 2.450 | 26.516 | 14.349 | 1.00 | 0.00 | 0.127 | 0.00 | 0.00 |
| ATOM | 1279 | CE1 | TYR | 78 | 5.503 | 27.374 | 12.197 | 1.00 | 0.00 | −0.127 | 10.80 | 0.60 |
| ATOM | 1280 | HE1 | TYR | 78 | 6.370 | 27.620 | 11.584 | 1.00 | 0.00 | 0.127 | 0.00 | 0.00 |
| ATOM | 1281 | CE2 | TYR | 78 | 4.551 | 26.255 | 14.067 | 1.00 | 0.00 | −0.127 | 10.80 | 0.60 |
| ATOM | 1282 | HE2 | TYR | 78 | 4.659 | 25.612 | 14.940 | 1.00 | 0.00 | 0.127 | 0.00 | 0.00 |
| ATOM | 1283 | CZ | TYR | 78 | 5.647 | 26.560 | 13.296 | 1.00 | 0.00 | 0.026 | 7.26 | 0.60 |
| ATOM | 1284 | OH | TYR | 78 | 6.915 | 26.040 | 13.635 | 1.00 | 0.00 | −0.451 | 10.94 | −17.40 |
| ATOM | 1285 | HH | TYR | 78 | 6.866 | 25.591 | 14.560 | 1.00 | 0.00 | 0.424 | 0.00 | 0.00 |
| ATOM | 1286 | N | SER | 79 | −0.497 | 28.677 | 10.263 | 1.00 | 0.00 | −0.650 | 9.00 | −17.40 |
| ATOM | 1287 | HN | SER | 79 | 0.281 | 29.128 | 9.762 | 1.00 | 0.00 | 0.440 | 0.00 | 0.00 |
| ATOM | 1288 | CA | SER | 79 | −1.835 | 29.159 | 10.088 | 1.00 | 0.00 | 0.158 | 9.40 | 4.00 |
| ATOM | 1289 | HA | SER | 79 | −2.275 | 29.367 | 11.062 | 1.00 | 0.00 | 0.053 | 0.00 | 0.00 |
| ATOM | 1290 | C | SER | 79 | −2.650 | 28.121 | 9.383 | 1.00 | 0.00 | 0.396 | 9.82 | 4.00 |
| ATOM | 1291 | O | SER | 79 | −3.791 | 27.855 | 9.761 | 1.00 | 0.00 | −0.396 | 8.17 | −17.40 |
| ATOM | 1292 | CB | SER | 79 | −1.899 | 30.449 | 9.251 | 1.00 | 0.00 | 0.007 | 12.77 | 4.00 |
| ATOM | 1293 | HB1 | SER | 79 | −2.938 | 30.730 | 9.081 | 1.00 | 0.00 | 0.053 | 0.00 | 0.00 |
| ATOM | 1294 | HB2 | SER | 79 | −1.412 | 30.292 | 8.288 | 1.00 | 0.00 | 0.053 | 0.00 | 0.00 |
| ATOM | 1295 | OG | SER | 79 | −1.240 | 31.504 | 9.936 | 1.00 | 0.00 | −0.537 | 11.04 | −17.40 |
| ATOM | 1296 | HG | SER | 79 | −0.359 | 31.730 | 9.452 | 1.00 | 0.00 | 0.424 | 0.00 | 0.00 |
| ATOM | 1297 | N | ILE | 80 | −2.070 | 27.484 | 8.349 | 1.00 | 0.00 | −0.650 | 9.00 | −17.40 |
| ATOM | 1298 | HN | ILE | 80 | −1.080 | 27.665 | 8.129 | 1.00 | 0.00 | 0.440 | 0.00 | 0.00 |
| ATOM | 1299 | CA | ILE | 80 | −2.815 | 26.555 | 7.550 | 1.00 | 0.00 | 0.158 | 9.40 | 4.00 |
| ATOM | 1300 | HA | ILE | 80 | −3.697 | 27.045 | 7.140 | 1.00 | 0.00 | 0.053 | 0.00 | 0.00 |
| ATOM | 1301 | C | ILE | 80 | −3.243 | 25.397 | 8.389 | 1.00 | 0.00 | 0.396 | 9.82 | 4.00 |
| ATOM | 1302 | O | ILE | 80 | −4.348 | 24.890 | 8.223 | 1.00 | 0.00 | −0.396 | 8.17 | −17.40 |
| ATOM | 1303 | CB | ILE | 80 | −2.053 | 26.034 | 6.359 | 1.00 | 0.00 | −0.053 | 9.40 | 4.00 |
| ATOM | 1304 | HB | ILE | 80 | −1.615 | 26.877 | 5.825 | 1.00 | 0.00 | 0.053 | 0.00 | 0.00 |
| ATOM | 1305 | CG1 | ILE | 80 | −3.013 | 25.367 | 5.360 | 1.00 | 0.00 | −0.106 | 12.77 | 4.00 |
| ATOM | 1306 | HG1 | ILE | 80 | −3.892 | 25.970 | 5.134 | 1.00 | 0.00 | 0.053 | 0.00 | 0.00 |
| ATOM | 1307 | HG1 | ILE | 80 | −3.403 | 24.410 | 5.706 | 1.00 | 0.00 | 0.053 | 0.00 | 0.00 |
| ATOM | 1308 | CG2 | ILE | 80 | −0.948 | 25.090 | 6.853 | 1.00 | 0.00 | −0.159 | 16.15 | 4.00 |
| ATOM | 1309 | HG2 | ILE | 80 | −0.389 | 24.706 | 5.999 | 1.00 | 0.00 | 0.053 | 0.00 | 0.00 |
| ATOM | 1310 | HG2 | ILE | 80 | −0.273 | 25.634 | 7.513 | 1.00 | 0.00 | 0.053 | 0.00 | 0.00 |
| ATOM | 1311 | HG2 | ILE | 80 | −1.396 | 24.258 | 7.397 | 1.00 | 0.00 | 0.053 | 0.00 | 0.00 |
| ATOM | 1312 | CD1 | ILE | 80 | −2.371 | 25.071 | 4.005 | 1.00 | 0.00 | −0.159 | 16.15 | 4.00 |
| ATOM | 1313 | HD1 | ILE | 80 | −3.104 | 24.601 | 3.349 | 1.00 | 0.00 | 0.053 | 0.00 | 0.00 |
| ATOM | 1314 | HD1 | ILE | 80 | −2.026 | 26.001 | 3.554 | 1.00 | 0.00 | 0.053 | 0.00 | 0.00 |
| ATOM | 1315 | HD1 | ILE | 80 | −1.524 | 24.398 | 4.142 | 1.00 | 0.00 | 0.053 | 0.00 | 0.00 |
| ATOM | 1316 | N | VAL | 81 | −2.390 | 24.924 | 9.311 | 1.00 | 0.00 | −0.650 | 9.00 | −17.40 |
| ATOM | 1317 | HN | VAL | 81 | −1.463 | 25.352 | 9.451 | 1.00 | 0.00 | 0.440 | 0.00 | 0.00 |
| ATOM | 1318 | CA | VAL | 81 | −2.815 | 23.801 | 10.093 | 1.00 | 0.00 | 0.158 | 9.40 | 4.00 |
| ATOM | 1319 | HA | VAL | 81 | −3.109 | 22.995 | 9.420 | 1.00 | 0.00 | 0.053 | 0.00 | 0.00 |
| ATOM | 1320 | C | VAL | 81 | −3.976 | 24.214 | 10.940 | 1.00 | 0.00 | 0.396 | 9.82 | 4.00 |
| ATOM | 1321 | O | VAL | 81 | −4.930 | 23.457 | 11.113 | 1.00 | 0.00 | −0.396 | 8.17 | −17.40 |
| ATOM | 1322 | CB | VAL | 81 | −1.746 | 23.252 | 10.997 | 1.00 | 0.00 | −0.053 | 9.40 | 4.00 |
| ATOM | 1323 | HB | VAL | 81 | −1.868 | 22.170 | 11.061 | 1.00 | 0.00 | 0.053 | 0.00 | 0.00 |
| ATOM | 1324 | CG1 | VAL | 81 | −0.373 | 23.601 | 10.399 | 1.00 | 0.00 | −0.159 | 16.15 | 4.00 |
| ATOM | 1325 | HG1 | VAL | 81 | −0.414 | 23.209 | 11.043 | 1.00 | 0.00 | 0.053 | 0.00 | 0.00 |
| ATOM | 1326 | HG1 | VAL | 81 | −0.284 | 23.157 | 9.407 | 1.00 | 0.00 | 0.053 | 0.00 | 0.00 |
| ATOM | 1327 | HG1 | VAL | 81 | −0.274 | 24.683 | 10.322 | 1.00 | 0.00 | 0.053 | 0.00 | 0.00 |
| ATOM | 1328 | CG2 | VAL | 81 | −1.956 | 23.799 | 12.416 | 1.00 | 0.00 | −0.159 | 16.15 | 4.00 |
| ATOM | 1329 | HG2 | VAL | 81 | −1.183 | 23.404 | 13.076 | 1.00 | 0.00 | 0.053 | 0.00 | 0.00 |
| ATOM | 1330 | HG2 | VAL | 81 | −1.897 | 24.887 | 12.398 | 1.00 | 0.00 | 0.053 | 0.00 | 0.00 |
| ATOM | 1331 | HG2 | VAL | 81 | −2.936 | 23.494 | 12.782 | 1.00 | 0.00 | 0.053 | 0.00 | 0.00 |
| ATOM | 1332 | N | VAL | 82 | −3.929 | 25.436 | 11.505 | 1.00 | 0.00 | −0.650 | 9.00 | −17.40 |
| ATOM | 1333 | HN | VAL | 82 | −3.137 | 26.065 | 11.308 | 1.00 | 0.00 | 0.440 | 0.00 | 0.00 |
| ATOM | 1334 | CA | VAL | 82 | −4.984 | 25.859 | 12.382 | 1.00 | 0.00 | 0.158 | 9.40 | 4.00 |
| ATOM | 1335 | HA | VAL | 82 | −5.043 | 25.173 | 13.227 | 1.00 | 0.00 | 0.053 | 0.00 | 0.00 |
| ATOM | 1336 | C | VAL | 82 | −6.276 | 25.852 | 11.634 | 1.00 | 0.00 | 0.396 | 9.82 | 4.00 |
| ATOM | 1337 | O | VAL | 82 | −7.251 | 25.239 | 12.066 | 1.00 | 0.00 | −0.396 | 8.17 | −17.40 |
| ATOM | 1338 | CB | VAL | 82 | −4.772 | 27.253 | 12.888 | 1.00 | 0.00 | −0.053 | 9.40 | 4.00 |
| ATOM | 1339 | HB | VAL | 82 | −4.713 | 27.922 | 12.029 | 1.00 | 0.00 | 0.053 | 0.00 | 0.00 |
| ATOM | 1340 | CG1 | VAL | 82 | −5.961 | 27.625 | 13.786 | 1.00 | 0.00 | −0.159 | 16.15 | 4.00 |
| ATOM | 1341 | HG1 | VAL | 82 | −5.827 | 28.637 | 14.166 | 1.00 | 0.00 | 0.053 | 0.00 | 0.00 |
| ATOM | 1342 | HG1 | VAL | 82 | −6.883 | 27.573 | 13.208 | 1.00 | 0.00 | 0.053 | 0.00 | 0.00 |
| ATOM | 1343 | HG1 | VAL | 82 | −6.017 | 26.928 | 14.622 | 1.00 | 0.00 | 0.053 | 0.00 | 0.00 |
| ATOM | 1344 | CG2 | VAL | 82 | −3.408 | 27.315 | 13.594 | 1.00 | 0.00 | −0.159 | 16.15 | 4.00 |
| ATOM | 1345 | HG2 | VAL | 82 | −3.237 | 28.324 | 13.968 | 1.00 | 0.00 | 0.053 | 0.00 | 0.00 |
| ATOM | 1346 | HG2 | VAL | 82 | −3.397 | 26.612 | 14.427 | 1.00 | 0.00 | 0.053 | 0.00 | 0.00 |
| ATOM | 1347 | HG2 | VAL | 82 | −2.620 | 27.052 | 12.887 | 1.00 | 0.00 | 0.053 | 0.00 | 0.00 |
| ATOM | 1348 | N | HIS | 83 | −6.294 | 26.505 | 10.462 | 1.00 | 0.00 | −0.650 | 9.00 | −17.40 |
| ATOM | 1349 | HN | HIS | 83 | −5.423 | 26.939 | 10.123 | 1.00 | 0.00 | 0.440 | 0.00 | 0.00 |
| ATOM | 1350 | CA | HIS | 83 | −7.475 | 26.620 | 9.665 | 1.00 | 0.00 | 0.158 | 9.40 | 4.00 |
| ATOM | 1351 | HA | HIS | 83 | −8.257 | 27.052 | 10.288 | 1.00 | 0.00 | 0.053 | 0.00 | 0.00 |
| ATOM | 1352 | C | HIS | 83 | −7.851 | 25.252 | 9.206 | 1.00 | 0.00 | 0.396 | 9.82 | 4.00 |
| ATOM | 1353 | O | HIS | 83 | −9.030 | 24.905 | 9.147 | 1.00 | 0.00 | −0.396 | 8.17 | −17.40 |
| ATOM | 1354 | CB | HIS | 83 | −7.236 | 27.515 | 8.431 | 1.00 | 0.00 | −0.106 | 12.77 | 4.00 |
| ATOM | 1355 | HB1 | HIS | 83 | −6.446 | 27.061 | 7.831 | 1.00 | 0.00 | 0.053 | 0.00 | 0.00 |
| ATOM | 1356 | HB2 | HIS | 83 | −6.937 | 28.502 | 8.781 | 1.00 | 0.00 | 0.053 | 0.00 | 0.00 |
| ATOM | 1357 | CG | HIS | 83 | −8.426 | 27.698 | 7.539 | 1.00 | 0.00 | −0.050 | 7.26 | 0.60 |
| ATOM | 1358 | ND1 | HIS | 83 | −8.576 | 27.085 | 6.316 | 1.00 | 0.00 | 0.207 | 9.25 | −17.40 |
| ATOM | 1359 | HD1 | HIS | 83 | −7.914 | 26.433 | 5.870 | 1.00 | 0.00 | 0.393 | 0.00 | 0.00 |
| ATOM | 1360 | CD2 | HIS | 83 | −9.541 | 28.461 | 7.710 | 1.00 | 0.00 | −0.177 | 10.80 | 0.60 |
| ATOM | 1361 | HD2 | HIS | 83 | −9.740 | 29.081 | 8.583 | 1.00 | 0.00 | 0.127 | 0.00 | 0.00 |
| ATOM | 1362 | CE1 | HIS | 83 | −9.763 | 27.503 | 5.810 | 1.00 | 0.00 | −0.227 | 10.80 | 0.60 |
| ATOM | 1363 | HE1 | HIS | 83 | −10.158 | 27.184 | 4.845 | 1.00 | 0.00 | 0.127 | 0.00 | 0.00 |
| ATOM | 1364 | NE2 | HIS | 83 | −10.385 | 28.340 | 6.621 | 1.00 | 0.00 | 0.207 | 9.25 | −17.40 |
| ATOM | 1365 | HE2 | HIS | 83 | −11.295 | 28.799 | 6.475 | 1.00 | 0.00 | 0.393 | 0.00 | 0.00 |
| ATOM | 1366 | N | HIS | 84 | −6.817 | 24.446 | 8.905 | 1.00 | 0.00 | −0.650 | 9.00 | −17.40 |
| ATOM | 1367 | HN | HIS | 84 | −5.875 | 24.826 | 9.075 | 1.00 | 0.00 | 0.440 | 0.00 | 0.00 |
| ATOM | 1368 | CA | HIS | 84 | −6.880 | 23.116 | 8.373 | 1.00 | 0.00 | 0.158 | 9.40 | 4.00 |
| ATOM | 1369 | HA | HIS | 84 | −7.038 | 23.156 | 7.295 | 1.00 | 0.00 | 0.053 | 0.00 | 0.00 |
| ATOM | 1370 | C | HIS | 84 | −8.004 | 22.374 | 9.003 | 1.00 | 0.00 | 0.396 | 9.82 | 4.00 |
| ATOM | 1371 | O | HIS | 84 | −8.285 | 22.515 | 10.193 | 1.00 | 0.00 | −0.396 | 8.17 | −17.40 |
| ATOM | 1372 | CB | HIS | 84 | −5.571 | 22.332 | 8.599 | 1.00 | 0.00 | −0.106 | 12.77 | 4.00 |
| ATOM | 1373 | HB1 | HIS | 84 | −4.900 | 22.958 | 9.187 | 1.00 | 0.00 | 0.053 | 0.00 | 0.00 |
| ATOM | 1374 | HB2 | HIS | 84 | −5.138 | 22.109 | 7.623 | 1.00 | 0.00 | 0.053 | 0.00 | 0.00 |
| ATOM | 1375 | CG | HIS | 84 | −5.716 | 21.027 | 9.329 | 1.00 | 0.00 | −0.050 | 7.26 | 0.60 |
| ATOM | 1376 | ND1 | HIS | 84 | −4.660 | 20.371 | 9.926 | 1.00 | 0.00 | 0.207 | 9.25 | −17.40 |
| ATOM | 1377 | HD1 | HIS | 84 | −3.677 | 20.680 | 9.945 | 1.00 | 0.00 | 0.393 | 0.00 | 0.00 |
| ATOM | 1378 | CD2 | HIS | 84 | −6.810 | 20.250 | 9.559 | 1.00 | 0.00 | −0.177 | 10.80 | 0.60 |
| ATOM | 1379 | HD2 | HIS | 84 | −7.819 | 20.482 | 9.218 | 1.00 | 0.00 | 0.127 | 0.00 | 0.00 |
| ATOM | 1380 | CE1 | HIS | 84 | −5.162 | 19.240 | 10.482 | 1.00 | 0.00 | −0.227 | 10.80 | 0.60 |
| ATOM | 1381 | HE1 | HIS | 84 | −4.561 | 18.511 | 11.027 | 1.00 | 0.00 | 0.127 | 0.00 | 0.00 |
| ATOM | 1382 | NE2 | HIS | 84 | −6.463 | 19.124 | 10.286 | 1.00 | 0.00 | 0.207 | 9.25 | −17.40 |
| ATOM | 1383 | HE2 | HIS | 84 | −7.082 | 18.364 | 10.603 | 1.00 | 0.00 | 0.393 | 0.00 | 0.00 |
| ATOM | 1384 | N | LYS | 85 | −8.699 | 21.562 | 8.189 | 1.00 | 0.00 | −0.650 | 9.00 | −17.40 |
| ATOM | 1385 | HN | LYS | 85 | −8.442 | 21.461 | 7.196 | 1.00 | 0.00 | 0.440 | 0.00 | 0.00 |
| ATOM | 1386 | CA | LYS | 85 | −9.802 | 20.844 | 8.737 | 1.00 | 0.00 | 0.158 | 9.40 | 4.00 |
| ATOM | 1387 | HA | LYS | 85 | −10.387 | 21.560 | 9.313 | 1.00 | 0.00 | 0.053 | 0.00 | 0.00 |
| ATOM | 1388 | C | LYS | 85 | −9.232 | 19.772 | 9.593 | 1.00 | 0.00 | 0.396 | 9.82 | 4.00 |
| ATOM | 1389 | O | LYS | 85 | −8.834 | 18.714 | 9.111 | 1.00 | 0.00 | −0.396 | 8.17 | −17.40 |
| ATOM | 1390 | CB | LYS | 85 | −10.702 | 20.146 | 7.707 | 1.00 | 0.00 | −0.106 | 12.77 | 4.00 |
| ATOM | 1391 | HB1 | LYS | 85 | −11.431 | 19.550 | 8.255 | 1.00 | 0.00 | 0.053 | 0.00 | 0.00 |
| ATOM | 1392 | HB2 | LYS | 85 | −10.065 | 19.516 | 7.085 | 1.00 | 0.00 | 0.053 | 0.00 | 0.00 |
| ATOM | 1393 | CG | LYS | 85 | −11.493 | 21.043 | 6.758 | 1.00 | 0.00 | −0.106 | 12.77 | 4.00 |
| ATOM | 1394 | HG1 | LYS | 85 | −10.848 | 21.683 | 6.155 | 1.00 | 0.00 | 0.053 | 0.00 | 0.00 |
| ATOM | 1395 | HG2 | LYS | 85 | −12.177 | 21.707 | 7.285 | 1.00 | 0.00 | 0.053 | 0.00 | 0.00 |
| ATOM | 1396 | CD | LYS | 85 | −12.345 | 20.245 | 5.774 | 1.00 | 0.00 | −0.106 | 12.77 | 4.00 |
| ATOM | 1397 | HD1 | LYS | 85 | −11.880 | 19.314 | 5.446 | 1.00 | 0.00 | 0.053 | 0.00 | 0.00 |
| ATOM | 1398 | HD2 | LYS | 85 | −12.572 | 20.784 | 4.854 | 1.00 | 0.00 | 0.053 | 0.00 | 0.00 |
| ATOM | 1399 | CE | LYS | 85 | −13.702 | 19.831 | 6.334 | 1.00 | 0.00 | 0.099 | 12.77 | 4.00 |
| ATOM | 1400 | HE1 | LYS | 85 | −13.573 | 19.289 | 7.271 | 1.00 | 0.00 | 0.053 | 0.00 | 0.00 |
| ATOM | 1401 | HE2 | LYS | 85 | −14.220 | 19.185 | 5.625 | 1.00 | 0.00 | 0.053 | 0.00 | 0.00 |
| ATOM | 1402 | NZ | LYS | 85 | −14.538 | 21.027 | 6.587 | 1.00 | 0.00 | −0.045 | 13.25 | −39.20 |
| ATOM | 1403 | HZ1 | LYS | 85 | −15.450 | 20.734 | 6.964 | 1.00 | 0.00 | 0.280 | 0.00 | 0.00 |
| ATOM | 1404 | HZ2 | LYS | 85 | −14.065 | 21.639 | 7.267 | 1.00 | 0.00 | 0.280 | 0.00 | 0.00 |
| ATOM | 1405 | HZ3 | LYS | 85 | −14.680 | 21.540 | 5.705 | 1.00 | 0.00 | 0.280 | 0.00 | 0.00 |
| ATOM | 1406 | N | ILE | 86 | −9.152 | 20.053 | 10.900 | 1.00 | 0.00 | −0.650 | 9.00 | −17.40 |
| ATOM | 1407 | HN | ILE | 86 | −9.444 | 20.986 | 11.223 | 1.00 | 0.00 | 0.440 | 0.00 | 0.00 |
| ATOM | 1408 | CA | ILE | 86 | −8.679 | 19.114 | 11.863 | 1.00 | 0.00 | 0.158 | 9.40 | 4.00 |
| ATOM | 1409 | HA | ILE | 86 | −8.832 | 18.132 | 11.413 | 1.00 | 0.00 | 0.053 | 0.00 | 0.00 |
| ATOM | 1410 | C | ILE | 86 | −9.510 | 19.336 | 13.078 | 1.00 | 0.00 | 0.396 | 9.82 | 4.00 |
| ATOM | 1411 | O | ILE | 86 | −9.993 | 20.444 | 13.312 | 1.00 | 0.00 | −0.396 | 8.17 | −17.40 |
| ATOM | 1412 | CB | ILE | 86 | −7.234 | 19.326 | 12.205 | 1.00 | 0.00 | −0.053 | 9.40 | 4.00 |
| ATOM | 1413 | HB | ILE | 86 | −6.633 | 18.994 | 11.357 | 1.00 | 0.00 | 0.053 | 0.00 | 0.00 |
| ATOM | 1414 | CG1 | ILE | 86 | −6.788 | 18.379 | 13.331 | 1.00 | 0.00 | −0.106 | 12.77 | 4.00 |
| ATOM | 1415 | HG1 | ILE | 86 | −7.054 | 17.337 | 13.152 | 1.00 | 0.00 | 0.053 | 0.00 | 0.00 |
| ATOM | 1416 | HG1 | ILE | 86 | −7.222 | 18.623 | 14.300 | 1.00 | 0.00 | 0.053 | 0.00 | 0.00 |
| ATOM | 1417 | CG2 | ILE | 86 | −7.022 | 20.822 | 12.472 | 1.00 | 0.00 | −0.159 | 16.15 | 4.00 |
| ATOM | 1418 | HG2 | ILE | 86 | −5.976 | 21.002 | 12.723 | 1.00 | 0.00 | 0.053 | 0.00 | 0.00 |
| ATOM | 1419 | HG2 | ILE | 86 | −7.284 | 21.391 | 11.580 | 1.00 | 0.00 | 0.053 | 0.00 | 0.00 |
| ATOM | 1420 | HG2 | ILE | 86 | −7.654 | 21.136 | 13.302 | 1.00 | 0.00 | 0.053 | 0.00 | 0.00 |
| ATOM | 1421 | CD1 | ILE | 86 | −5.278 | 18.374 | 13.557 | 1.00 | 0.00 | −0.159 | 16.15 | 4.00 |
| ATOM | 1422 | HD1 | ILE | 86 | −5.033 | 17.684 | 14.365 | 1.00 | 0.00 | 0.053 | 0.00 | 0.00 |
| ATOM | 1423 | HD1 | ILE | 86 | −4.774 | 18.056 | 12.644 | 1.00 | 0.00 | 0.053 | 0.00 | 0.00 |
| ATOM | 1424 | HD1 | ILE | 86 | −4.946 | 19.377 | 13.823 | 1.00 | 0.00 | 0.053 | 0.00 | 0.00 |
| ATOM | 1425 | N | GLU | 87 | −9.731 | 18.276 | 13.875 | 1.00 | 0.00 | −0.650 | 9.00 | −17.40 |
| ATOM | 1426 | HN | GLU | 87 | −9.339 | 17.348 | 13.657 | 1.00 | 0.00 | 0.440 | 0.00 | 0.00 |
| ATOM | 1427 | CA | GLU | 87 | −10.532 | 18.493 | 15.036 | 1.00 | 0.00 | 0.158 | 9.40 | 4.00 |
| ATOM | 1428 | HA | GLU | 87 | −11.492 | 18.876 | 14.692 | 1.00 | 0.00 | 0.053 | 0.00 | 0.00 |
| ATOM | 1429 | C | GLU | 87 | −9.814 | 19.478 | 15.879 | 1.00 | 0.00 | 0.396 | 9.82 | 4.00 |
| ATOM | 1430 | O | GLU | 87 | −10.377 | 20.470 | 16.339 | 1.00 | 0.00 | −0.396 | 8.17 | −17.40 |
| ATOM | 1431 | CB | GLU | 87 | −10.698 | 17.282 | 15.963 | 1.00 | 0.00 | −0.106 | 12.77 | 4.00 |
| ATOM | 1432 | HB1 | GLU | 87 | −9.729 | 16.946 | 16.334 | 1.00 | 0.00 | 0.053 | 0.00 | 0.00 |
| ATOM | 1433 | HB2 | GLU | 87 | −11.166 | 16.453 | 15.431 | 1.00 | 0.00 | 0.053 | 0.00 | 0.00 |
| ATOM | 1434 | CG | GLU | 87 | −11.577 | 17.640 | 17.167 | 1.00 | 0.00 | −0.106 | 12.77 | 4.00 |
| ATOM | 1435 | HG1 | GLU | 87 | −12.628 | 17.543 | 16.896 | 1.00 | 0.00 | 0.053 | 0.00 | 0.00 |
| ATOM | 1436 | HG2 | GLU | 87 | −11.378 | 18.666 | 17.475 | 1.00 | 0.00 | 0.053 | 0.00 | 0.00 |
| ATOM | 1437 | CD | GLU | 87 | −11.269 | 16.700 | 18.326 | 1.00 | 0.00 | 0.399 | 9.82 | 4.00 |
| ATOM | 1438 | OE1 | GLU | 87 | −10.462 | 15.754 | 18.132 | 1.00 | 0.00 | −0.396 | 8.17 | −18.95 |
| ATOM | 1439 | OE2 | GLU | 87 | −11.831 | 16.924 | 19.430 | 1.00 | 0.00 | −0.427 | 8.17 | −18.95 |
| ATOM | 1441 | N | MET | 88 | −8.517 | 19.230 | 16.103 | 1.00 | 0.00 | −0.650 | 9.00 | −17.40 |
| ATOM | 1442 | HN | MET | 88 | −8.034 | 18.435 | 15.659 | 1.00 | 0.00 | 0.440 | 0.00 | 0.00 |
| ATOM | 1443 | CA | MET | 88 | −7.840 | 20.117 | 16.983 | 1.00 | 0.00 | 0.158 | 9.40 | 4.00 |
| ATOM | 1444 | HA | MET | 88 | −8.363 | 20.130 | 17.939 | 1.00 | 0.00 | 0.053 | 0.00 | 0.00 |
| ATOM | 1445 | C | MET | 88 | −7.836 | 21.477 | 16.383 | 1.00 | 0.00 | 0.396 | 9.82 | 4.00 |
| ATOM | 1446 | O | MET | 88 | −8.324 | 22.426 | 16.996 | 1.00 | 0.00 | −0.396 | 8.17 | −17.40 |
| ATOM | 1447 | CB | MET | 88 | −6.386 | 19.710 | 17.261 | 1.00 | 0.00 | −0.106 | 12.77 | 4.00 |
| ATOM | 1448 | HB1 | MET | 88 | −5.738 | 19.871 | 16.399 | 1.00 | 0.00 | 0.053 | 0.00 | 0.00 |
| ATOM | 1449 | HB2 | MET | 88 | −6.293 | 18.656 | 17.523 | 1.00 | 0.00 | 0.053 | 0.00 | 0.00 |
| ATOM | 1450 | CG | MET | 88 | −5.773 | 20.499 | 18.417 | 1.00 | 0.00 | −0.041 | 12.77 | 4.00 |
| ATOM | 1451 | HG1 | MET | 88 | −6.410 | 20.370 | 19.292 | 1.00 | 0.00 | 0.053 | 0.00 | 0.00 |
| ATOM | 1452 | HG2 | MET | 88 | −5.724 | 21.548 | 18.125 | 1.00 | 0.00 | 0.053 | 0.00 | 0.00 |
| ATOM | 1453 | SD | MET | 88 | −4.097 | 19.985 | 18.883 | 1.00 | 0.00 | −0.130 | 16.39 | −6.40 |
| ATOM | 1454 | CE | MET | 88 | −4.082 | 20.930 | 20.434 | 1.00 | 0.00 | −0.094 | 16.15 | 4.00 |
| ATOM | 1455 | HE1 | MET | 88 | −3.127 | 20.784 | 20.939 | 1.00 | 0.00 | 0.053 | 0.00 | 0.00 |
| ATOM | 1456 | HE2 | MET | 88 | −4.219 | 21.989 | 20.216 | 1.00 | 0.00 | 0.053 | 0.00 | 0.00 |
| ATOM | 1457 | HE3 | MET | 88 | −4.890 | 20.584 | 21.078 | 1.00 | 0.00 | 0.053 | 0.00 | 0.00 |
| ATOM | 1458 | N | LEU | 89 | −7.309 | 21.623 | 15.154 | 1.00 | 0.00 | −0.650 | 9.00 | −17.40 |
| ATOM | 1459 | HN | LEU | 89 | −6.991 | 20.839 | 14.566 | 1.00 | 0.00 | 0.440 | 0.00 | 0.00 |
| ATOM | 1460 | CA | LEU | 89 | −7.243 | 22.984 | 14.765 | 1.00 | 0.00 | 0.158 | 9.40 | 4.00 |
| ATOM | 1461 | HA | LEU | 89 | −6.805 | 23.554 | 15.584 | 1.00 | 0.00 | 0.053 | 0.00 | 0.00 |
| ATOM | 1462 | C | LEU | 89 | −8.576 | 23.594 | 14.444 | 1.00 | 0.00 | 0.396 | 9.82 | 4.00 |
| ATOM | 1463 | O | LEU | 89 | −8.964 | 24.519 | 15.145 | 1.00 | 0.00 | −0.396 | 8.17 | −17.40 |
| ATOM | 1464 | CB | LEU | 89 | −6.109 | 23.308 | 13.769 | 1.00 | 0.00 | −0.106 | 12.77 | 4.00 |
| ATOM | 1465 | HB1 | LEU | 89 | −6.197 | 24.353 | 13.474 | 1.00 | 0.00 | 0.053 | 0.00 | 0.00 |
| ATOM | 1466 | HB2 | LEU | 89 | −6.215 | 22.656 | 12.901 | 1.00 | 0.00 | 0.053 | 0.00 | 0.00 |
| ATOM | 1467 | CG | LEU | 89 | −4.748 | 23.055 | 14.475 | 1.00 | 0.00 | −0.053 | 9.40 | 4.00 |
| ATOM | 1468 | HG | LEU | 89 | −4.705 | 23.557 | 15.441 | 1.00 | 0.00 | 0.053 | 0.00 | 0.00 |
| ATOM | 1469 | CD1 | LEU | 89 | −4.518 | 21.556 | 14.719 | 1.00 | 0.00 | −0.159 | 16.15 | 4.00 |
| ATOM | 1470 | HD1 | LEU | 89 | −3.557 | 21.411 | 15.214 | 1.00 | 0.00 | 0.053 | 0.00 | 0.00 |
| ATOM | 1471 | HD1 | LEU | 89 | −5.314 | 21.163 | 15.350 | 1.00 | 0.00 | 0.053 | 0.00 | 0.00 |
| ATOM | 1472 | HD1 | LEU | 89 | −4.517 | 21.028 | 13.765 | 1.00 | 0.00 | 0.053 | 0.00 | 0.00 |
| ATOM | 1473 | CD2 | LEU | 89 | −3.544 | 23.728 | 13.804 | 1.00 | 0.00 | −0.159 | 16.15 | 4.00 |
| ATOM | 1474 | HD2 | LEU | 89 | −2.638 | 23.496 | 14.364 | 1.00 | 0.00 | 0.053 | 0.00 | 0.00 |
| ATOM | 1475 | HD2 | LEU | 89 | −3.442 | 23.358 | 12.783 | 1.00 | 0.00 | 0.053 | 0.00 | 0.00 |
| ATOM | 1476 | HD2 | LEU | 89 | −3.693 | 24.807 | 13.786 | 1.00 | 0.00 | 0.053 | 0.00 | 0.00 |
| ATOM | 1477 | N | ASP | 90 | −9.399 | 23.087 | 13.502 | 1.00 | 0.00 | −0.650 | 9.00 | −17.40 |
| ATOM | 1478 | HN | ASP | 90 | −9.222 | 22.212 | 12.987 | 1.00 | 0.00 | 0.440 | 0.00 | 0.00 |
| ATOM | 1479 | CA | ASP | 90 | −10.555 | 23.924 | 13.307 | 1.00 | 0.00 | 0.158 | 9.40 | 4.00 |
| ATOM | 1480 | HA | ASP | 90 | −11.000 | 24.273 | 14.238 | 1.00 | 0.00 | 0.053 | 0.00 | 0.00 |
| ATOM | 1481 | C | ASP | 90 | −11.653 | 23.252 | 12.581 | 1.00 | 0.00 | 0.396 | 9.82 | 4.00 |
| ATOM | 1482 | O | ASP | 90 | −12.629 | 22.789 | 13.169 | 1.00 | 0.00 | −0.396 | 8.17 | −17.40 |
| ATOM | 1483 | CB | ASP | 90 | −10.305 | 25.181 | 12.452 | 1.00 | 0.00 | −0.336 | 12.77 | 4.00 |
| ATOM | 1484 | HB1 | ASP | 90 | −11.213 | 25.510 | 11.948 | 1.00 | 0.00 | 0.053 | 0.00 | 0.00 |
| ATOM | 1485 | HB2 | ASP | 90 | −9.555 | 24.999 | 11.681 | 1.00 | 0.00 | 0.053 | 0.00 | 0.00 |
| ATOM | 1486 | CG | ASP | 90 | −9.812 | 26.339 | 13.295 | 1.00 | 0.00 | 0.297 | 9.82 | 4.00 |
| ATOM | 1487 | OD1 | ASP | 90 | −10.432 | 26.555 | 14.368 | 1.00 | 0.00 | −0.534 | 8.17 | −18.95 |
| ATOM | 1488 | OD2 | ASP | 90 | −8.852 | 27.041 | 12.878 | 1.00 | 0.00 | −0.534 | 8.17 | −18.95 |
| ATOM | 1489 | N | ARG | 91 | −11.508 | 23.231 | 11.251 | 1.00 | 0.00 | −0.650 | 9.00 | −17.40 |
| ATOM | 1490 | HN | ARG | 91 | −10.596 | 23.497 | 10.852 | 1.00 | 0.00 | 0.440 | 0.00 | 0.00 |
| ATOM | 1491 | CA | ARG | 91 | −12.556 | 22.857 | 10.359 | 1.00 | 0.00 | 0.158 | 9.40 | 4.00 |
| ATOM | 1492 | HA | ARG | 91 | −13.431 | 23.501 | 10.431 | 1.00 | 0.00 | 0.053 | 0.00 | 0.00 |
| ATOM | 1493 | C | ARG | 91 | −13.059 | 21.474 | 10.592 | 1.00 | 0.00 | 0.396 | 9.82 | 4.00 |
| ATOM | 1494 | O | ARG | 91 | −14.265 | 21.280 | 10.702 | 1.00 | 0.00 | −0.396 | 8.17 | −17.40 |
| ATOM | 1495 | CB | ARG | 91 | −12.110 | 22.979 | 8.895 | 1.00 | 0.00 | −0.106 | 12.77 | 4.00 |
| ATOM | 1496 | HB1 | ARG | 91 | −12.758 | 22.418 | 8.221 | 1.00 | 0.00 | 0.053 | 0.00 | 0.00 |
| ATOM | 1497 | HB2 | ARG | 91 | −11.098 | 22.603 | 8.742 | 1.00 | 0.00 | 0.053 | 0.00 | 0.00 |
| ATOM | 1498 | CG | ARG | 91 | −12.119 | 24.424 | 8.412 | 1.00 | 0.00 | −0.106 | 12.77 | 4.00 |
| ATOM | 1499 | HG1 | ARG | 91 | −11.348 | 24.586 | 7.658 | 1.00 | 0.00 | 0.053 | 0.00 | 0.00 |
| ATOM | 1500 | HG2 | ARG | 91 | −11.933 | 25.110 | 9.237 | 1.00 | 0.00 | 0.053 | 0.00 | 0.00 |
| ATOM | 1501 | CD | ARG | 91 | −13.466 | 24.782 | 7.794 | 1.00 | 0.00 | 0.374 | 12.77 | 4.00 |
| ATOM | 1502 | HD1 | ARG | 91 | −14.236 | 24.660 | 8.555 | 1.00 | 0.00 | 0.053 | 0.00 | 0.00 |
| ATOM | 1503 | HD2 | ARG | 91 | −13.648 | 24.110 | 6.955 | 1.00 | 0.00 | 0.053 | 0.00 | 0.00 |
| ATOM | 1504 | NE | ARG | 91 | −13.408 | 26.191 | 7.331 | 1.00 | 0.00 | −0.819 | 9.00 | −24.67 |
| ATOM | 1505 | HE | ARG | 91 | −12.585 | 26.779 | 7.525 | 1.00 | 0.00 | 0.407 | 0.00 | 0.00 |
| ATOM | 1506 | CZ | ARG | 91 | −14.476 | 26.683 | 6.641 | 1.00 | 0.00 | 0.796 | 6.95 | 4.00 |
| ATOM | 1507 | NH1 | ARG | 91 | −15.554 | 25.881 | 6.402 | 1.00 | 0.00 | −0.746 | 9.00 | −24.67 |
| ATOM | 1508 | HH1 | ARG | 91 | −16.363 | 26.247 | 5.881 | 1.00 | 0.00 | 0.407 | 0.00 | 0.00 |
| ATOM | 1509 | HH1 | ARG | 91 | −15.558 | 24.908 | 6.742 | 1.00 | 0.00 | 0.407 | 0.00 | 0.00 |
| ATOM | 1510 | NH2 | ARG | 91 | −14.470 | 27.975 | 6.200 | 1.00 | 0.00 | −0.746 | 9.00 | −24.67 |
| ATOM | 1511 | HH2 | ARG | 91 | −15.277 | 28.345 | 5.678 | 1.00 | 0.00 | 0.407 | 0.00 | 0.00 |
| ATOM | 1512 | HH2 | ARG | 91 | −13.657 | 28.579 | 6.388 | 1.00 | 0.00 | 0.407 | 0.00 | 0.00 |
| ATOM | 1513 | N | LEU | 92 | −12.191 | 20.458 | 10.694 | 1.00 | 0.00 | −0.650 | 9.00 | −17.40 |
| ATOM | 1514 | HN | LEU | 92 | −11.169 | 20.588 | 10.695 | 1.00 | 0.00 | 0.440 | 0.00 | 0.00 |
| ATOM | 1515 | CA | LEU | 92 | −12.816 | 19.177 | 10.800 | 1.00 | 0.00 | 0.158 | 9.40 | 4.00 |
| ATOM | 1516 | HA | LEU | 92 | −13.545 | 19.006 | 10.008 | 1.00 | 0.00 | 0.053 | 0.00 | 0.00 |
| ATOM | 1517 | C | LEU | 92 | −13.540 | 19.009 | 12.084 | 1.00 | 0.00 | 0.396 | 9.82 | 4.00 |
| ATOM | 1518 | O | LEU | 92 | −14.591 | 18.370 | 12.102 | 1.00 | 0.00 | −0.396 | 8.17 | −17.40 |
| ATOM | 1519 | CB | LEU | 92 | −11.955 | 17.931 | 10.520 | 1.00 | 0.00 | −0.106 | 12.77 | 4.00 |
| ATOM | 1520 | HB1 | LEU | 92 | −11.105 | 17.879 | 11.200 | 1.00 | 0.00 | 0.053 | 0.00 | 0.00 |
| ATOM | 1521 | HB2 | LEU | 92 | −11.565 | 17.943 | 9.502 | 1.00 | 0.00 | 0.053 | 0.00 | 0.00 |
| ATOM | 1522 | CG | LEU | 92 | −12.783 | 16.627 | 10.695 | 1.00 | 0.00 | −0.053 | 9.40 | 4.00 |
| ATOM | 1523 | HG | LEU | 92 | −13.816 | 16.764 | 10.377 | 1.00 | 0.00 | 0.053 | 0.00 | 0.00 |
| ATOM | 1524 | CD1 | LEU | 92 | −12.201 | 15.479 | 9.870 | 1.00 | 0.00 | −0.159 | 16.15 | 4.00 |
| ATOM | 1525 | HD1 | LEU | 92 | −12.804 | 14.583 | 10.016 | 1.00 | 0.00 | 0.053 | 0.00 | 0.00 |
| ATOM | 1526 | HD1 | LEU | 92 | −12.205 | 15.750 | 8.814 | 1.00 | 0.00 | 0.053 | 0.00 | 0.00 |
| ATOM | 1527 | HD1 | LEU | 92 | −11.177 | 15.283 | 10.190 | 1.00 | 0.00 | 0.053 | 0.00 | 0.00 |
| ATOM | 1528 | CD2 | LEU | 92 | −12.866 | 16.169 | 12.166 | 1.00 | 0.00 | −0.159 | 16.15 | 4.00 |
| ATOM | 1529 | HD2 | LEU | 92 | −13.455 | 15.254 | 12.229 | 1.00 | 0.00 | 0.053 | 0.00 | 0.00 |
| ATOM | 1530 | HD2 | LEU | 92 | −11.861 | 15.980 | 12.545 | 1.00 | 0.00 | 0.053 | 0.00 | 0.00 |
| ATOM | 1531 | HD2 | LEU | 92 | −13.339 | 16.948 | 12.763 | 1.00 | 0.00 | 0.053 | 0.00 | 0.00 |
| ATOM | 1532 | N | LEU | 93 | −13.017 | 19.556 | 13.191 | 1.00 | 0.00 | −0.650 | 9.00 | −17.40 |
| ATOM | 1533 | HN | LEU | 93 | −12.165 | 20.133 | 13.142 | 1.00 | 0.00 | 0.440 | 0.00 | 0.00 |
| ATOM | 1534 | CA | LEU | 93 | −13.673 | 19.316 | 14.444 | 1.00 | 0.00 | 0.158 | 9.40 | 4.00 |
| ATOM | 1535 | HA | LEU | 93 | −13.691 | 18.238 | 14.606 | 1.00 | 0.00 | 0.053 | 0.00 | 0.00 |
| ATOM | 1536 | C | LEU | 93 | −15.047 | 19.868 | 14.345 | 1.00 | 0.00 | 0.396 | 9.82 | 4.00 |
| ATOM | 1537 | O | LEU | 93 | −16.023 | 19.224 | 14.733 | 1.00 | 0.00 | −0.396 | 8.17 | −17.40 |
| ATOM | 1538 | CB | LEU | 93 | −12.962 | 20.049 | 15.595 | 1.00 | 0.00 | −0.106 | 12.77 | 4.00 |
| ATOM | 1539 | HB1 | LEU | 93 | −13.013 | 21.116 | 15.379 | 1.00 | 0.00 | 0.053 | 0.00 | 0.00 |
| ATOM | 1540 | HB2 | LEU | 93 | −11.931 | 19.693 | 15.617 | 1.00 | 0.00 | 0.053 | 0.00 | 0.00 |
| ATOM | 1541 | CG | LEU | 93 | −13.510 | 19.873 | 17.030 | 1.00 | 0.00 | −0.053 | 9.40 | 4.00 |
| ATOM | 1542 | HG | LEU | 93 | −13.616 | 18.814 | 17.265 | 1.00 | 0.00 | 0.053 | 0.00 | 0.00 |
| ATOM | 1543 | CD1 | LEU | 93 | −12.548 | 20.511 | 18.049 | 1.00 | 0.00 | −0.159 | 16.15 | 4.00 |
| ATOM | 1544 | HD1 | LEU | 93 | −12.945 | 20.380 | 19.055 | 1.00 | 0.00 | 0.053 | 0.00 | 0.00 |
| ATOM | 1545 | HD1 | LEU | 93 | −11.572 | 20.030 | 17.978 | 1.00 | 0.00 | 0.053 | 0.00 | 0.00 |
| ATOM | 1546 | HD1 | LEU | 93 | −12.444 | 21.574 | 17.835 | 1.00 | 0.00 | 0.053 | 0.00 | 0.00 |
| ATOM | 1547 | CD2 | LEU | 93 | −14.924 | 20.449 | 17.207 | 1.00 | 0.00 | −0.159 | 16.15 | 4.00 |
| ATOM | 1548 | HD2 | LEU | 93 | −15.253 | 20.295 | 18.234 | 1.00 | 0.00 | 0.053 | 0.00 | 0.00 |
| ATOM | 1549 | HD2 | LEU | 93 | −14.912 | 21.516 | 16.985 | 1.00 | 0.00 | 0.053 | 0.00 | 0.00 |
| ATOM | 1550 | HD2 | LEU | 93 | −15.610 | 19.944 | 16.526 | 1.00 | 0.00 | 0.053 | 0.00 | 0.00 |
| ATOM | 1551 | N | ARG | 94 | −15.136 | 21.098 | 13.826 | 1.00 | 0.00 | −0.650 | 9.00 | −17.40 |
| ATOM | 1552 | HN | ARG | 94 | −14.276 | 21.575 | 13.517 | 1.00 | 0.00 | 0.440 | 0.00 | 0.00 |
| ATOM | 1553 | CA | ARG | 94 | −16.388 | 21.766 | 13.688 | 1.00 | 0.00 | 0.158 | 9.40 | 4.00 |
| ATOM | 1554 | HA | ARG | 94 | −16.858 | 21.773 | 14.671 | 1.00 | 0.00 | 0.053 | 0.00 | 0.00 |
| ATOM | 1555 | C | ARG | 94 | −17.191 | 20.995 | 12.700 | 1.00 | 0.00 | 0.396 | 9.82 | 4.00 |
| ATOM | 1556 | O | ARG | 94 | −18.409 | 20.883 | 12.821 | 1.00 | 0.00 | −0.396 | 8.17 | −17.40 |
| ATOM | 1557 | CB | ARG | 94 | −16.204 | 23.205 | 13.181 | 1.00 | 0.00 | −0.106 | 12.77 | 4.00 |
| ATOM | 1558 | HB1 | ARG | 94 | −17.163 | 23.706 | 13.053 | 1.00 | 0.00 | 0.053 | 0.00 | 0.00 |
| ATOM | 1559 | HB2 | ARG | 94 | −15.692 | 23.224 | 12.218 | 1.00 | 0.00 | 0.053 | 0.00 | 0.00 |
| ATOM | 1560 | CG | ARG | 94 | −15.375 | 24.039 | 14.160 | 1.00 | 0.00 | −0.106 | 12.77 | 4.00 |
| ATOM | 1561 | HG1 | ARG | 94 | −14.677 | 24.647 | 13.584 | 1.00 | 0.00 | 0.053 | 0.00 | 0.00 |
| ATOM | 1562 | HG2 | ARG | 94 | −14.837 | 23.357 | 14.819 | 1.00 | 0.00 | 0.053 | 0.00 | 0.00 |
| ATOM | 1563 | CD | ARG | 94 | −16.193 | 24.984 | 15.041 | 1.00 | 0.00 | 0.374 | 12.77 | 4.00 |
| ATOM | 1564 | HD1 | ARG | 94 | −15.691 | 25.052 | 16.006 | 1.00 | 0.00 | 0.053 | 0.00 | 0.00 |
| ATOM | 1565 | HD2 | ARG | 94 | −17.191 | 24.558 | 15.144 | 1.00 | 0.00 | 0.053 | 0.00 | 0.00 |
| ATOM | 1566 | NE | ARG | 94 | −16.239 | 26.307 | 14.362 | 1.00 | 0.00 | −0.819 | 9.00 | −24.67 |
| ATOM | 1567 | HE | ARG | 94 | −16.878 | 26.467 | 13.570 | 1.00 | 0.00 | 0.407 | 0.00 | 0.00 |
| ATOM | 1568 | CZ | ARG | 94 | −15.420 | 27.303 | 14.805 | 1.00 | 0.00 | 0.796 | 6.95 | 4.00 |
| ATOM | 1569 | NH1 | ARG | 94 | −14.626 | 27.078 | 15.893 | 1.00 | 0.00 | −0.746 | 9.00 | −24.67 |
| ATOM | 1570 | HH1 | ARG | 94 | −14.002 | 27.821 | 16.238 | 1.00 | 0.00 | 0.407 | 0.00 | 0.00 |
| ATOM | 1571 | HH1 | ARG | 94 | −14.650 | 26.164 | 16.368 | 1.00 | 0.00 | 0.407 | 0.00 | 0.00 |
| ATOM | 1572 | NH2 | ARG | 94 | −15.421 | 28.523 | 14.191 | 1.00 | 0.00 | −0.746 | 9.00 | −24.67 |
| ATOM | 1573 | HH2 | ARG | 94 | −14.799 | 29.271 | 14.530 | 1.00 | 0.00 | 0.407 | 0.00 | 0.00 |
| ATOM | 1574 | HH2 | ARG | 94 | −16.042 | 28.695 | 13.388 | 1.00 | 0.00 | 0.407 | 0.00 | 0.00 |
| ATOM | 1575 | N | ALA | 95 | −16.528 | 20.440 | 11.680 | 1.00 | 0.00 | −0.650 | 9.00 | −17.40 |
| ATOM | 1576 | HN | ALA | 95 | −15.506 | 20.544 | 11.602 | 1.00 | 0.00 | 0.440 | 0.00 | 0.00 |
| ATOM | 1577 | CA | ALA | 95 | −17.255 | 19.703 | 10.703 | 1.00 | 0.00 | 0.158 | 9.40 | 4.00 |
| ATOM | 1578 | HA | ALA | 95 | −18.019 | 20.339 | 10.257 | 1.00 | 0.00 | 0.053 | 0.00 | 0.00 |
| ATOM | 1579 | C | ALA | 95 | −17.908 | 18.526 | 11.346 | 1.00 | 0.00 | 0.396 | 9.82 | 4.00 |
| ATOM | 1580 | O | ALA | 95 | −19.133 | 18.402 | 11.333 | 1.00 | 0.00 | −0.396 | 8.17 | −17.40 |
| ATOM | 1581 | CB | ALA | 95 | −16.337 | 19.153 | 9.608 | 1.00 | 0.00 | −0.159 | 16.15 | 4.00 |
| ATOM | 1582 | HB1 | ALA | 95 | −16.929 | 18.596 | 8.881 | 1.00 | 0.00 | 0.053 | 0.00 | 0.00 |
| ATOM | 1583 | HB2 | ALA | 95 | −15.832 | 19.979 | 9.107 | 1.00 | 0.00 | 0.053 | 0.00 | 0.00 |
| ATOM | 1584 | HB3 | ALA | 95 | −15.594 | 18.490 | 10.054 | 1.00 | 0.00 | 0.053 | 0.00 | 0.00 |
| ATOM | 1585 | N | ILE | 96 | −17.109 | 17.637 | 11.965 | 1.00 | 0.00 | −0.650 | 9.00 | −17.40 |
| ATOM | 1586 | HN | ILE | 96 | −16.094 | 17.794 | 12.047 | 1.00 | 0.00 | 0.440 | 0.00 | 0.00 |
| ATOM | 1587 | CA | ILE | 96 | −17.725 | 16.468 | 12.502 | 1.00 | 0.00 | 0.158 | 9.40 | 4.00 |
| ATOM | 1588 | HA | ILE | 96 | −18.342 | 15.982 | 11.746 | 1.00 | 0.00 | 0.053 | 0.00 | 0.00 |
| ATOM | 1589 | C | ILE | 96 | −18.585 | 16.821 | 13.658 | 1.00 | 0.00 | 0.396 | 9.82 | 4.00 |
| ATOM | 1590 | O | ILE | 96 | −19.729 | 16.375 | 13.729 | 1.00 | 0.00 | −0.396 | 8.17 | −17.40 |
| ATOM | 1591 | CB | ILE | 96 | −16.770 | 15.390 | 12.900 | 1.00 | 0.00 | −0.053 | 9.40 | 4.00 |
| ATOM | 1592 | HB | ILE | 96 | −15.944 | 15.850 | 13.443 | 1.00 | 0.00 | 0.053 | 0.00 | 0.00 |
| ATOM | 1593 | CG1 | ILE | 96 | −16.195 | 14.755 | 11.624 | 1.00 | 0.00 | −0.106 | 12.77 | 4.00 |
| ATOM | 1594 | HG1 | ILE | 96 | −15.698 | 15.536 | 11.048 | 1.00 | 0.00 | 0.053 | 0.00 | 0.00 |
| ATOM | 1595 | HG1 | ILE | 96 | −17.019 | 14.323 | 11.056 | 1.00 | 0.00 | 0.053 | 0.00 | 0.00 |
| ATOM | 1596 | CG2 | ILE | 96 | −17.520 | 14.391 | 13.795 | 1.00 | 0.00 | −0.159 | 16.15 | 4.00 |
| ATOM | 1597 | HG2 | ILE | 96 | −16.841 | 13.593 | 14.098 | 1.00 | 0.00 | 0.053 | 0.00 | 0.00 |
| ATOM | 1598 | HG2 | ILE | 96 | −17.894 | 14.905 | 14.680 | 1.00 | 0.00 | 0.053 | 0.00 | 0.00 |
| ATOM | 1599 | HG2 | ILE | 96 | −18.357 | 13.964 | 13.242 | 1.00 | 0.00 | 0.053 | 0.00 | 0.00 |
| ATOM | 1600 | CD1 | ILE | 96 | −15.178 | 13.649 | 11.869 | 1.00 | 0.00 | −0.159 | 16.15 | 4.00 |
| ATOM | 1601 | HD1 | ILE | 96 | −14.826 | 13.259 | 10.913 | 1.00 | 0.00 | 0.053 | 0.00 | 0.00 |
| ATOM | 1602 | HD1 | ILE | 96 | −14.333 | 14.048 | 12.430 | 1.00 | 0.00 | 0.053 | 0.00 | 0.00 |
| ATOM | 1603 | HD1 | ILE | 96 | −15.644 | 12.845 | 12.438 | 1.00 | 0.00 | 0.053 | 0.00 | 0.00 |
| ATOM | 1604 | N | TYR | 97 | −18.101 | 17.641 | 14.611 | 1.00 | 0.00 | −0.650 | 9.00 | −17.40 |
| ATOM | 1605 | HN | TYR | 97 | −17.170 | 18.083 | 14.614 | 1.00 | 0.00 | 0.440 | 0.00 | 0.00 |
| ATOM | 1606 | CA | TYR | 97 | −19.100 | 17.787 | 15.609 | 1.00 | 0.00 | 0.158 | 9.40 | 4.00 |
| ATOM | 1607 | HA | TYR | 97 | −20.023 | 17.342 | 15.237 | 1.00 | 0.00 | 0.053 | 0.00 | 0.00 |
| ATOM | 1608 | C | TYR | 97 | −19.393 | 19.195 | 15.976 | 1.00 | 0.00 | 0.396 | 9.82 | 4.00 |
| ATOM | 1609 | O | TYR | 97 | −19.384 | 19.554 | 17.155 | 1.00 | 0.00 | −0.396 | 8.17 | −17.40 |
| ATOM | 1610 | CB | TYR | 97 | −18.921 | 16.914 | 16.868 | 1.00 | 0.00 | −0.106 | 12.77 | 4.00 |
| ATOM | 1611 | HB1 | TYR | 97 | −18.666 | 15.876 | 16.649 | 1.00 | 0.00 | 0.053 | 0.00 | 0.00 |
| ATOM | 1612 | HB2 | TYR | 97 | −19.812 | 16.858 | 17.493 | 1.00 | 0.00 | 0.053 | 0.00 | 0.00 |
| ATOM | 1613 | CG | TYR | 97 | −17.847 | 17.388 | 17.775 | 1.00 | 0.00 | 0.000 | 7.26 | 0.60 |
| ATOM | 1614 | CD1 | TYR | 97 | −16.514 | 17.320 | 17.452 | 1.00 | 0.00 | −0.127 | 10.80 | 0.60 |
| ATOM | 1615 | HD1 | TYR | 97 | −16.205 | 16.913 | 16.488 | 1.00 | 0.00 | 0.127 | 0.00 | 0.00 |
| ATOM | 1616 | CD2 | TYR | 97 | −18.217 | 17.877 | 19.003 | 1.00 | 0.00 | −0.127 | 10.80 | 0.60 |
| ATOM | 1617 | HD2 | TYR | 97 | −19.273 | 17.913 | 19.267 | 1.00 | 0.00 | 0.127 | 0.00 | 0.00 |
| ATOM | 1618 | CE1 | TYR | 97 | −15.569 | 17.765 | 18.345 | 1.00 | 0.00 | −0.127 | 10.80 | 0.60 |
| ATOM | 1619 | HE1 | TYR | 97 | −14.512 | 17.724 | 18.081 | 1.00 | 0.00 | 0.127 | 0.00 | 0.00 |
| ATOM | 1620 | CE2 | TYR | 97 | −17.282 | 18.321 | 19.903 | 1.00 | 0.00 | −0.127 | 10.80 | 0.60 |
| ATOM | 1621 | HE2 | TYR | 97 | −17.591 | 18.715 | 20.870 | 1.00 | 0.00 | 0.127 | 0.00 | 0.00 |
| ATOM | 1622 | CZ | TYR | 97 | −15.951 | 18.262 | 19.570 | 1.00 | 0.00 | 0.026 | 7.26 | 0.60 |
| ATOM | 1623 | OH | TYR | 97 | −14.976 | 18.717 | 20.483 | 1.00 | 0.00 | −0.451 | 10.94 | −17.40 |
| ATOM | 1624 | HH | TYR | 97 | −14.425 | 19.472 | 20.050 | 1.00 | 0.00 | 0.424 | 0.00 | 0.00 |
| ATOM | 1625 | N | MET | 98 | −19.659 | 20.055 | 14.974 | 1.00 | 0.00 | −0.650 | 9.00 | −17.40 |
| ATOM | 1626 | HN | MET | 98 | −19.406 | 19.908 | 13.986 | 1.00 | 0.00 | 0.440 | 0.00 | 0.00 |
| ATOM | 1627 | CA | MET | 98 | −20.337 | 21.192 | 15.480 | 1.00 | 0.00 | 0.158 | 9.40 | 4.00 |
| ATOM | 1628 | HA | MET | 98 | −19.879 | 21.619 | 16.372 | 1.00 | 0.00 | 0.053 | 0.00 | 0.00 |
| ATOM | 1629 | C | MET | 98 | −21.570 | 20.420 | 15.683 | 1.00 | 0.00 | 0.396 | 9.82 | 4.00 |
| ATOM | 1630 | O | MET | 98 | −22.221 | 20.066 | 14.701 | 1.00 | 0.00 | −0.396 | 8.17 | −17.40 |
| ATOM | 1631 | CB | MET | 98 | −20.624 | 22.327 | 14.481 | 1.00 | 0.00 | −0.106 | 12.77 | 4.00 |
| ATOM | 1632 | HB1 | MET | 98 | −21.295 | 22.015 | 13.680 | 1.00 | 0.00 | 0.053 | 0.00 | 0.00 |
| ATOM | 1633 | HB2 | MET | 98 | −19.718 | 22.693 | 13.998 | 1.00 | 0.00 | 0.053 | 0.00 | 0.00 |
| ATOM | 1634 | CG | MET | 98 | −21.281 | 23.542 | 15.150 | 1.00 | 0.00 | −0.041 | 12.77 | 4.00 |
| ATOM | 1635 | HG1 | MET | 98 | −20.623 | 23.884 | 15.948 | 1.00 | 0.00 | 0.053 | 0.00 | 0.00 |
| ATOM | 1636 | HG2 | MET | 98 | −22.245 | 23.227 | 15.549 | 1.00 | 0.00 | 0.053 | 0.00 | 0.00 |
| ATOM | 1637 | SD | MET | 98 | −21.587 | 24.962 | 14.054 | 1.00 | 0.00 | −0.130 | 16.39 | −6.40 |
| ATOM | 1638 | CE | MET | 98 | −19.832 | 25.393 | 13.879 | 1.00 | 0.00 | −0.094 | 16.15 | 4.00 |
| ATOM | 1639 | HE1 | MET | 98 | −19.734 | 26.264 | 13.231 | 1.00 | 0.00 | 0.053 | 0.00 | 0.00 |
| ATOM | 1640 | HE2 | MET | 98 | −19.413 | 25.621 | 14.859 | 1.00 | 0.00 | 0.053 | 0.00 | 0.00 |
| ATOM | 1641 | HE3 | MET | 98 | −19.292 | 24.552 | 13.441 | 1.00 | 0.00 | 0.053 | 0.00 | 0.00 |
| ATOM | 1642 | N | PRO | 99 | −21.869 | 20.163 | 16.924 | 1.00 | 0.00 | −0.422 | 9.00 | −17.40 |
| ATOM | 1643 | CA | PRO | 99 | −22.771 | 19.112 | 17.318 | 1.00 | 0.00 | 0.158 | 9.40 | 4.00 |
| ATOM | 1644 | HA | PRO | 99 | −22.323 | 18.118 | 17.286 | 1.00 | 0.00 | 0.053 | 0.00 | 0.00 |
| ATOM | 1645 | CD | PRO | 99 | −21.854 | 21.240 | 17.900 | 1.00 | 0.00 | 0.105 | 12.77 | 4.00 |
| ATOM | 1646 | HD1 | PRO | 99 | −22.029 | 22.150 | 17.327 | 1.00 | 0.00 | 0.053 | 0.00 | 0.00 |
| ATOM | 1647 | HD2 | PRO | 99 | −20.864 | 21.202 | 18.356 | 1.00 | 0.00 | 0.053 | 0.00 | 0.00 |
| ATOM | 1648 | C | PRO | 99 | −23.980 | 19.017 | 16.465 | 1.00 | 0.00 | 0.396 | 9.82 | 4.00 |
| ATOM | 1649 | O | PRO | 99 | −24.463 | 17.900 | 16.286 | 1.00 | 0.00 | −0.396 | 8.17 | −17.40 |
| ATOM | 1650 | CD | PRO | 99 | −23.152 | 19.390 | 18.768 | 1.00 | 0.00 | −0.106 | 12.77 | 4.00 |
| ATOM | 1651 | HB1 | PRO | 99 | −22.494 | 18.857 | 19.455 | 1.00 | 0.00 | 0.053 | 0.00 | 0.00 |
| ATOM | 1652 | HB2 | PRO | 99 | −24.174 | 19.073 | 18.972 | 1.00 | 0.00 | 0.053 | 0.00 | 0.00 |
| ATOM | 1653 | CG | PRO | 99 | −22.990 | 20.913 | 18.885 | 1.00 | 0.00 | −0.106 | 12.77 | 4.00 |
| ATOM | 1654 | HG1 | PRO | 99 | −22.731 | 21.199 | 19.904 | 1.00 | 0.00 | 0.053 | 0.00 | 0.00 |
| ATOM | 1655 | HG2 | PRO | 99 | −23.914 | 21.424 | 18.615 | 1.00 | 0.00 | 0.053 | 0.00 | 0.00 |
| ATOM | 1656 | N | GLN | 100 | −24.520 | 20.135 | 15.947 | 1.00 | 0.00 | −0.650 | 9.00 | −17.40 |
| ATOM | 1657 | HN | GLN | 100 | −24.187 | 21.084 | 16.168 | 1.00 | 0.00 | 0.440 | 0.00 | 0.00 |
| ATOM | 1658 | CA | GLN | 100 | −25.605 | 19.864 | 15.056 | 1.00 | 0.00 | 0.158 | 9.40 | 4.00 |
| ATOM | 1659 | HA | GLN | 100 | −26.143 | 19.023 | 15.492 | 1.00 | 0.00 | 0.053 | 0.00 | 0.00 |
| ATOM | 1660 | C | GLN | 100 | −24.977 | 19.533 | 13.741 | 1.00 | 0.00 | 0.396 | 9.82 | 4.00 |
| ATOM | 1661 | O | GLN | 100 | −24.377 | 20.385 | 13.094 | 1.00 | 0.00 | −0.396 | 8.17 | −17.40 |
| ATOM | 1662 | CB | GLN | 100 | −26.671 | 20.978 | 14.860 | 1.00 | 0.00 | −0.106 | 12.77 | 4.00 |
| ATOM | 1663 | HB1 | GLN | 100 | −27.029 | 21.273 | 15.846 | 1.00 | 0.00 | 0.053 | 0.00 | 0.00 |
| ATOM | 1664 | HB2 | GLN | 100 | −27.479 | 20.563 | 14.257 | 1.00 | 0.00 | 0.053 | 0.00 | 0.00 |
| ATOM | 1665 | CG | GLN | 100 | −26.198 | 22.249 | 14.158 | 1.00 | 0.00 | −0.106 | 12.77 | 4.00 |
| ATOM | 1666 | HG1 | GLN | 100 | −25.587 | 21.956 | 13.304 | 1.00 | 0.00 | 0.053 | 0.00 | 0.00 |
| ATOM | 1667 | HG2 | GLN | 100 | −25.614 | 22.831 | 14.870 | 1.00 | 0.00 | 0.053 | 0.00 | 0.00 |
| ATOM | 1668 | CD | GLN | 100 | −27.427 | 23.023 | 13.704 | 1.00 | 0.00 | 0.396 | 9.82 | 4.00 |
| ATOM | 1669 | OE1 | GLN | 100 | −27.352 | 24.224 | 13.453 | 1.00 | 0.00 | −0.396 | 8.17 | −17.40 |
| ATOM | 1670 | NE2 | GLN | 100 | −28.583 | 22.314 | 13.579 | 1.00 | 0.00 | −0.879 | 13.25 | −17.40 |
| ATOM | 1671 | HE2 | GLN | 100 | −28.596 | 21.308 | 13.801 | 1.00 | 0.00 | 0.440 | 0.00 | 0.00 |
| ATOM | 1672 | HE2 | GLN | 100 | −29.443 | 22.783 | 13.262 | 1.00 | 0.00 | 0.440 | 0.00 | 0.00 |
| ATOM | 1673 | N | ASN | 101 | −25.087 | 18.255 | 13.337 | 1.00 | 0.00 | −0.650 | 9.00 | −17.40 |
| ATOM | 1674 | HN | ASN | 101 | −25.644 | 17.627 | 13.933 | 1.00 | 0.00 | 0.440 | 0.00 | 0.00 |
| ATOM | 1675 | CA | ASN | 101 | −24.504 | 17.675 | 12.155 | 1.00 | 0.00 | 0.158 | 9.40 | 4.00 |
| ATOM | 1676 | HA | ASN | 101 | −25.087 | 17.919 | 11.267 | 1.00 | 0.00 | 0.053 | 0.00 | 0.00 |
| ATOM | 1677 | C | ASN | 101 | −24.531 | 16.227 | 12.454 | 1.00 | 0.00 | 0.396 | 9.82 | 4.00 |
| ATOM | 1678 | O | ASN | 101 | −25.536 | 15.562 | 12.216 | 1.00 | 0.00 | −0.396 | 8.17 | −17.40 |
| ATOM | 1679 | CB | ASN | 101 | −23.016 | 18.027 | 11.940 | 1.00 | 0.00 | −0.106 | 12.77 | 4.00 |
| ATOM | 1680 | HB1 | ASN | 101 | −22.478 | 17.767 | 12.852 | 1.00 | 0.00 | 0.053 | 0.00 | 0.00 |
| ATOM | 1681 | HB2 | ASN | 101 | −22.954 | 19.096 | 11.737 | 1.00 | 0.00 | 0.053 | 0.00 | 0.00 |
| ATOM | 1682 | CG | ASN | 101 | −22.520 | 17.223 | 10.771 | 1.00 | 0.00 | 0.396 | 9.82 | 4.00 |
| ATOM | 1683 | OD1 | ASN | 101 | −23.202 | 17.163 | 9.758 | 1.00 | 0.00 | −0.396 | 8.17 | −17.40 |
| ATOM | 1684 | ND2 | ASN | 101 | −21.325 | 16.573 | 10.903 | 1.00 | 0.00 | −0.879 | 13.25 | −17.40 |
| ATOM | 1685 | HD2 | ASN | 101 | −20.787 | 16.655 | 11.777 | 1.00 | 0.00 | 0.440 | 0.00 | 0.00 |
| ATOM | 1686 | HD2 | ASN | 101 | −20.961 | 16.000 | 10.127 | 1.00 | 0.00 | 0.440 | 0.00 | 0.00 |
| ATOM | 1687 | N | PHE | 102 | −23.406 | 15.736 | 13.016 | 1.00 | 0.00 | −0.650 | 9.00 | −17.40 |
| ATOM | 1688 | HN | PHE | 102 | −22.614 | 16.380 | 13.150 | 1.00 | 0.00 | 0.440 | 0.00 | 0.00 |
| ATOM | 1689 | CA | PHE | 102 | −23.242 | 14.380 | 13.437 | 1.00 | 0.00 | 0.158 | 9.40 | 4.00 |
| ATOM | 1690 | HA | PHE | 102 | −24.136 | 13.797 | 13.217 | 1.00 | 0.00 | 0.053 | 0.00 | 0.00 |
| ATOM | 1691 | C | PHE | 102 | −22.988 | 14.310 | 14.913 | 1.00 | 0.00 | 0.396 | 9.82 | 4.00 |
| ATOM | 1692 | O | PHE | 102 | −23.162 | 15.289 | 15.635 | 1.00 | 0.00 | −0.396 | 8.17 | −17.40 |
| ATOM | 1693 | CB | PHE | 102 | −22.192 | 13.582 | 12.654 | 1.00 | 0.00 | −0.106 | 12.77 | 4.00 |
| ATOM | 1694 | HB1 | PHE | 102 | −21.692 | 12.858 | 13.298 | 1.00 | 0.00 | 0.053 | 0.00 | 0.00 |
| ATOM | 1695 | HB2 | PHE | 102 | −21.431 | 14.242 | 12.237 | 1.00 | 0.00 | 0.053 | 0.00 | 0.00 |
| ATOM | 1696 | CG | PHE | 102 | −22.985 | 12.911 | 11.584 | 1.00 | 0.00 | 0.000 | 7.26 | 0.60 |
| ATOM | 1697 | CD1 | PHE | 102 | −23.496 | 13.618 | 10.521 | 1.00 | 0.00 | −0.127 | 10.80 | 0.60 |
| ATOM | 1698 | HD1 | PHE | 102 | −23.307 | 14.688 | 10.439 | 1.00 | 0.00 | 0.127 | 0.00 | 0.00 |
| ATOM | 1699 | CD2 | PHE | 102 | −23.266 | 11.565 | 11.686 | 1.00 | 0.00 | −0.127 | 10.80 | 0.60 |
| ATOM | 1700 | HD2 | PHE | 102 | −22.893 | 11.000 | 12.541 | 1.00 | 0.00 | 0.127 | 0.00 | 0.00 |
| ATOM | 1701 | CE1 | PHE | 102 | −24.244 | 12.978 | 9.561 | 1.00 | 0.00 | −0.127 | 10.80 | 0.60 |
| ATOM | 1702 | HE1 | PHE | 102 | −24.637 | 13.544 | 8.716 | 1.00 | 0.00 | 0.127 | 0.00 | 0.00 |
| ATOM | 1703 | CE2 | PHE | 102 | −24.010 | 10.925 | 10.722 | 1.00 | 0.00 | −0.127 | 10.80 | 0.60 |
| ATOM | 1704 | HE2 | PHE | 102 | −24.209 | 9.856 | 10.805 | 1.00 | 0.00 | 0.127 | 0.00 | 0.00 |
| ATOM | 1705 | CZ | PHE | 102 | −24.500 | 11.635 | 9.656 | 1.00 | 0.00 | −0.127 | 10.80 | 0.60 |
| ATOM | 1706 | HZ | PHE | 102 | −25.089 | 11.133 | 8.888 | 1.00 | 0.00 | 0.127 | 0.00 | 0.00 |
| ATOM | 1707 | N | TYR | 103 | −22.546 | 13.120 | 15.381 | 1.00 | 0.00 | −0.650 | 9.00 | −17.40 |
| ATOM | 1708 | HN | TYR | 103 | −22.218 | 12.425 | 14.694 | 1.00 | 0.00 | 0.440 | 0.00 | 0.00 |
| ATOM | 1709 | CA | TYR | 103 | −22.503 | 12.759 | 16.778 | 1.00 | 0.00 | 0.158 | 9.40 | 4.00 |
| ATOM | 1710 | HA | TYR | 103 | −23.379 | 13.198 | 17.253 | 1.00 | 0.00 | 0.053 | 0.00 | 0.00 |
| ATOM | 1711 | C | TYR | 103 | −21.239 | 13.314 | 17.341 | 1.00 | 0.00 | 0.396 | 9.82 | 4.00 |
| ATOM | 1712 | O | TYR | 103 | −20.501 | 14.025 | 16.663 | 1.00 | 0.00 | −0.396 | 8.17 | −17.40 |
| ATOM | 1713 | CB | TYR | 103 | −22.207 | 11.275 | 17.012 | 1.00 | 0.00 | −0.106 | 12.77 | 4.00 |
| ATOM | 1714 | HB1 | TYR | 103 | −22.977 | 10.867 | 17.666 | 1.00 | 0.00 | 0.053 | 0.00 | 0.00 |
| ATOM | 1715 | HB2 | TYR | 103 | −21.226 | 11.187 | 17.479 | 1.00 | 0.00 | 0.053 | 0.00 | 0.00 |
| ATOM | 1716 | CG | TYR | 103 | −22.220 | 10.578 | 15.699 | 1.00 | 0.00 | 0.000 | 7.26 | 0.60 |
| ATOM | 1717 | CD1 | TYR | 103 | −21.039 | 10.654 | 14.996 | 1.00 | 0.00 | −0.127 | 10.80 | 0.60 |
| ATOM | 1718 | HD1 | TYR | 103 | −20.200 | 11.198 | 15.428 | 1.00 | 0.00 | 0.127 | 0.00 | 0.00 |
| ATOM | 1719 | CD2 | TYR | 103 | −23.269 | 9.861 | 15.167 | 1.00 | 0.00 | −0.127 | 10.80 | 0.60 |
| ATOM | 1720 | HD2 | TYR | 103 | −24.205 | 9.764 | 15.716 | 1.00 | 0.00 | 0.127 | 0.00 | 0.00 |
| ATOM | 1721 | CE1 | TYR | 103 | −20.887 | 10.066 | 13.770 | 1.00 | 0.00 | −0.127 | 10.80 | 0.60 |
| ATOM | 1722 | HE1 | TYR | 103 | −19.946 | 10.156 | 13.227 | 1.00 | 0.00 | 0.127 | 0.00 | 0.00 |
| ATOM | 1723 | CE2 | TYR | 103 | −23.121 | 9.266 | 13.929 | 1.00 | 0.00 | −0.127 | 10.80 | 0.60 |
| ATOM | 1724 | HE2 | TYR | 103 | −23.953 | 8.712 | 13.495 | 1.00 | 0.00 | 0.127 | 0.00 | 0.00 |
| ATOM | 1725 | CZ | TYR | 103 | −21.932 | 9.363 | 13.234 | 1.00 | 0.00 | 0.026 | 7.26 | 0.60 |
| ATOM | 1726 | OH | TYR | 103 | −21.779 | 8.754 | 11.968 | 1.00 | 0.00 | −0.451 | 10.94 | −17.40 |
| ATOM | 1727 | HH | TYR | 103 | −22.160 | 9.374 | 11.239 | 1.00 | 0.00 | 0.424 | 0.00 | 0.00 |
| ATOM | 1728 | N | CYS | 104S | −20.960 | 13.030 | 18.637 | 1.00 | 0.00 | −0.65 | 9.00 | −17.40 |
| ATOM | 1729 | HN | CYS | 104S | −21.648 | 12.656 | 19.306 | 1.00 | 0.00 | 0.44 | 0.00 | 0.00 |
| ATOM | 1730 | CA | CYS | 104S | −19.600 | 13.310 | 18.963 | 1.00 | 0.00 | 0.15 | 9.40 | 4.00 |
| ATOM | 1731 | HA | CYS | 104S | −18.984 | 12.952 | 18.137 | 1.00 | 0.00 | 0.05 | 0.00 | 0.00 |
| ATOM | 1732 | C | CYS | 104S | −19.048 | 12.690 | 20.204 | 1.00 | 0.00 | 0.39 | 9.82 | 4.00 |
| ATOM | 1733 | O | CYS | 104S | −19.521 | 12.835 | 21.331 | 1.00 | 0.00 | −0.39 | 8.17 | −17.40 |
| ATOM | 1734 | CB | CYS | 104S | −19.118 | 14.756 | 18.939 | 1.00 | 0.00 | −0.04 | 12.77 | 4.00 |
| ATOM | 1735 | HB1 | CYS | 104S | −19.572 | 15.230 | 19.809 | 1.00 | 0.00 | 0.05 | 0.00 | 0.00 |
| ATOM | 1736 | HB2 | CYS | 104S | −19.473 | 15.167 | 17.994 | 1.00 | 0.00 | 0.05 | 0.00 | 0.00 |
| ATOM | 1737 | SG | CYS | 104S | −17.299 | 14.667 | 19.041 | 1.00 | 0.00 | −0.25 | 19.93 | −6.40 |
| ATOM | 1738 | HG | CYS | 104S | −16.914 | 14.817 | 20.315 | 1.00 | 0.00 | 0.19 | 0.00 | 0.00 |
| ATOM | 1739 | N | VAL | 105 | −17.992 | 11.928 | 19.907 | 1.00 | 0.00 | −0.650 | 9.00 | −17.40 |
| ATOM | 1740 | HN | VAL | 105 | −17.911 | 11.723 | 18.900 | 1.00 | 0.00 | 0.440 | 0.00 | 0.00 |
| ATOM | 1741 | CA | VAL | 105 | −16.958 | 11.340 | 20.679 | 1.00 | 0.00 | 0.158 | 9.40 | 4.00 |
| ATOM | 1742 | HA | VAL | 105 | −16.647 | 11.954 | 21.524 | 1.00 | 0.00 | 0.053 | 0.00 | 0.00 |
| ATOM | 1743 | C | VAL | 105 | −15.992 | 11.281 | 19.541 | 1.00 | 0.00 | 0.396 | 9.82 | 4.00 |
| ATOM | 1744 | O | VAL | 105 | −16.389 | 10.948 | 18.425 | 1.00 | 0.00 | −0.396 | 8.17 | −17.40 |
| ATOM | 1745 | CB | VAL | 105 | −17.249 | 9.958 | 21.211 | 1.00 | 0.00 | −0.053 | 9.40 | 4.00 |
| ATOM | 1746 | HB | VAL | 105 | −18.193 | 9.964 | 21.754 | 1.00 | 0.00 | 0.053 | 0.00 | 0.00 |
| ATOM | 1747 | CG1 | VAL | 105 | −17.344 | 8.963 | 20.046 | 1.00 | 0.00 | −0.159 | 16.15 | 4.00 |
| ATOM | 1748 | HG1 | VAL | 105 | −17.554 | 7.966 | 20.434 | 1.00 | 0.00 | 0.053 | 0.00 | 0.00 |
| ATOM | 1749 | HG1 | VAL | 105 | −18.145 | 9.266 | 19.372 | 1.00 | 0.00 | 0.053 | 0.00 | 0.00 |
| ATOM | 1750 | HG1 | VAL | 105 | −16.399 | 8.948 | 19.502 | 1.00 | 0.00 | 0.053 | 0.00 | 0.00 |
| ATOM | 1751 | CG2 | VAL | 105 | −16.146 | 9.605 | 22.224 | 1.00 | 0.00 | −0.159 | 16.15 | 4.00 |
| ATOM | 1752 | HG2 | VAL | 105 | −16.328 | 8.609 | 22.627 | 1.00 | 0.00 | 0.053 | 0.00 | 0.00 |
| ATOM | 1753 | HG2 | VAL | 105 | −15.176 | 9.623 | 21.726 | 1.00 | 0.00 | 0.053 | 0.00 | 0.00 |
| ATOM | 1754 | HG2 | VAL | 105 | −16.151 | 10.332 | 23.035 | 1.00 | 0.00 | 0.053 | 0.00 | 0.00 |
| ATOM | 1755 | N | HIS | 106S | −14.723 | 11.667 | 19.721 | 1.00 | 0.00 | −0.650 | 9.00 | −17.40 |
| ATOM | 1756 | HN | HIS | 106S | −14.309 | 11.878 | 20.640 | 1.00 | 0.00 | 0.440 | 0.00 | 0.00 |
| ATOM | 1757 | CA | HIS | 106S | −14.009 | 11.751 | 18.485 | 1.00 | 0.00 | 0.158 | 9.40 | 4.00 |
| ATOM | 1758 | HA | HIS | 106S | −14.361 | 10.960 | 17.822 | 1.00 | 0.00 | 0.053 | 0.00 | 0.00 |
| ATOM | 1759 | C | HIS | 106S | −12.557 | 11.582 | 18.759 | 1.00 | 0.00 | 0.396 | 9.82 | 4.00 |
| ATOM | 1760 | O | HIS | 106S | −12.015 | 12.193 | 19.677 | 1.00 | 0.00 | −0.396 | 8.17 | −17.40 |
| ATOM | 1761 | CB | HIS | 106S | −14.260 | 13.143 | 17.895 | 1.00 | 0.00 | −0.106 | 12.77 | 4.00 |
| ATOM | 1762 | HB1 | HIS | 106S | −13.689 | 13.859 | 18.486 | 1.00 | 0.00 | 0.053 | 0.00 | 0.00 |
| ATOM | 1763 | HB2 | HIS | 106S | −15.330 | 13.340 | 17.959 | 1.00 | 0.00 | 0.053 | 0.00 | 0.00 |
| ATOM | 1764 | CG | HIS | 106S | −13.872 | 13.354 | 16.479 | 1.00 | 0.00 | −0.050 | 7.26 | 0.60 |
| ATOM | 1765 | ND1 | HIS | 106S | −12.959 | 14.299 | 16.079 | 1.00 | 0.00 | 0.207 | 9.25 | −17.40 |
| ATOM | 1766 | HD1 | HIS | 106S | −12.420 | 14.932 | 16.687 | 1.00 | 0.00 | 0.393 | 0.00 | 0.00 |
| ATOM | 1767 | CD2 | HIS | 106S | −14.324 | 12.757 | 15.345 | 1.00 | 0.00 | −0.177 | 10.80 | 0.60 |
| ATOM | 1768 | HD2 | HIS | 106S | −15.062 | 11.955 | 15.313 | 1.00 | 0.00 | 0.127 | 0.00 | 0.00 |
| ATOM | 1769 | CE1 | HIS | 106S | −12.906 | 14.235 | 14.729 | 1.00 | 0.00 | −0.227 | 10.80 | 0.60 |
| ATOM | 1770 | HE1 | HIS | 106S | −12.269 | 14.872 | 14.115 | 1.00 | 0.00 | 0.127 | 0.00 | 0.00 |
| ATOM | 1771 | NE2 | HIS | 106S | −13.713 | 13.312 | 14.239 | 1.00 | 0.00 | 0.207 | 9.25 | −17.40 |
| ATOM | 1772 | HE2 | HIS | 106S | −13.855 | 13.061 | 13.250 | 1.00 | 0.00 | 0.393 | 0.00 | 0.00 |
| ATOM | 1773 | N | VAL | 107 | −11.880 | 10.731 | 17.964 | 1.00 | 0.00 | −0.650 | 9.00 | −17.40 |
| ATOM | 1774 | HN | VAL | 107 | −12.354 | 10.197 | 17.221 | 1.00 | 0.00 | 0.440 | 0.00 | 0.00 |
| ATOM | 1775 | CA | VAL | 107 | −10.476 | 10.599 | 18.194 | 1.00 | 0.00 | 0.158 | 9.40 | 4.00 |
| ATOM | 1776 | HA | VAL | 107 | −10.246 | 11.079 | 19.145 | 1.00 | 0.00 | 0.053 | 0.00 | 0.00 |
| ATOM | 1777 | C | VAL | 107 | −9.764 | 11.270 | 17.070 | 1.00 | 0.00 | 0.396 | 9.82 | 4.00 |
| ATOM | 1778 | O | VAL | 107 | −9.843 | 10.855 | 15.915 | 1.00 | 0.00 | −0.396 | 8.17 | −17.40 |
| ATOM | 1779 | CB | VAL | 107 | −9.996 | 9.181 | 18.312 | 1.00 | 0.00 | −0.053 | 9.40 | 4.00 |
| ATOM | 1780 | HB | VAL | 107 | −8.908 | 9.204 | 18.382 | 1.00 | 0.00 | 0.053 | 0.00 | 0.00 |
| ATOM | 1781 | CG1 | VAL | 107 | −10.622 | 8.581 | 19.579 | 1.00 | 0.00 | −0.159 | 16.15 | 4.00 |
| ATOM | 1782 | HG1 | VAL | 107 | −10.292 | 7.548 | 19.693 | 1.00 | 0.00 | 0.053 | 0.00 | 0.00 |
| ATOM | 1783 | HG1 | VAL | 107 | −10.310 | 9.160 | 20.448 | 1.00 | 0.00 | 0.053 | 0.00 | 0.00 |
| ATOM | 1784 | HG1 | VAL | 107 | −11.708 | 8.607 | 19.495 | 1.00 | 0.00 | 0.053 | 0.00 | 0.00 |
| ATOM | 1785 | CG2 | VAL | 107 | −10.352 | 8.402 | 17.039 | 1.00 | 0.00 | −0.159 | 16.15 | 4.00 |
| ATOM | 1786 | HG2 | VAL | 107 | −10.001 | 17.374 | 17.131 | 1.00 | 0.00 | 0.053 | 0.00 | 0.00 |
| ATOM | 1787 | HG2 | VAL | 107 | −11.433 | 8.405 | 16.900 | 1.00 | 0.00 | 0.053 | 0.00 | 0.00 |
| ATOM | 1788 | HG2 | VAL | 107 | −9.875 | 8.872 | 16.179 | 1.00 | 0.00 | 0.053 | 0.00 | 0.00 |
| ATOM | 1789 | N | ASP | 108P | −9.045 | 12.353 | 17.414 | 1.00 | 0.00 | −0.650 | 9.00 | −17.40 |
| ATOM | 1790 | HN | ASP | 108P | −9.017 | 12.629 | 18.405 | 1.00 | 0.00 | 0.440 | 0.00 | 0.00 |
| ATOM | 1791 | CA | ASP | 108P | −8.317 | 13.138 | 16.466 | 1.00 | 0.00 | 0.158 | 9.40 | 4.00 |
| ATOM | 1792 | HA | ASP | 108P | −8.741 | 12.931 | 15.483 | 1.00 | 0.00 | 0.053 | 0.00 | 0.00 |
| ATOM | 1793 | C | ASP | 108P | −6.898 | 12.719 | 16.548 | 1.00 | 0.00 | 0.396 | 9.82 | 4.00 |
| ATOM | 1794 | O | ASP | 108P | −6.395 | 12.370 | 17.615 | 1.00 | 0.00 | −0.396 | 8.17 | −17.40 |
| ATOM | 1795 | CB | ASP | 108P | −8.363 | 14.646 | 16.769 | 1.00 | 0.00 | −0.336 | 12.77 | 4.00 |
| ATOM | 1796 | HB1 | ASP | 108P | −8.022 | 14.818 | 17.790 | 1.00 | 0.00 | 0.053 | 0.00 | 0.00 |
| ATOM | 1797 | HB2 | ASP | 108P | −9.386 | 15.004 | 16.658 | 1.00 | 0.00 | 0.053 | 0.00 | 0.00 |
| ATOM | 1798 | CG | ASP | 108P | −7.455 | 15.382 | 15.796 | 1.00 | 0.00 | 0.297 | 9.82 | 4.00 |
| ATOM | 1799 | OD1 | ASP | 108P | −6.210 | 15.308 | 15.971 | 1.00 | 0.00 | −0.534 | 8.17 | −18.95 |
| ATOM | 1800 | OD2 | ASP | 108P | −8.000 | 16.042 | 14.872 | 1.00 | 0.00 | −0.534 | 8.17 | −18.95 |
| ATOM | 1801 | N | THR | 109 | −6.207 | 12.700 | 15.400 | 1.00 | 0.00 | −0.650 | 9.00 | −17.40 |
| ATOM | 1802 | HN | THR | 109 | −6.629 | 12.929 | 14.489 | 1.00 | 0.00 | 0.440 | 0.00 | 0.00 |
| ATOM | 1803 | CA | THR | 109 | −4.844 | 12.337 | 15.546 | 1.00 | 0.00 | 0.158 | 9.40 | 4.00 |
| ATOM | 1804 | HA | THR | 109 | −4.701 | 11.939 | 16.550 | 1.00 | 0.00 | 0.053 | 0.00 | 0.00 |
| ATOM | 1805 | C | THR | 109 | −4.010 | 13.556 | 15.340 | 1.00 | 0.00 | 0.396 | 9.82 | 4.00 |
| ATOM | 1806 | O | THR | 109 | −3.820 | 14.003 | 14.208 | 1.00 | 0.00 | −0.396 | 8.17 | −17.40 |
| ATOM | 1807 | CB | THR | 109 | −4.390 | 11.274 | 14.594 | 1.00 | 0.00 | 0.060 | 9.40 | 4.00 |
| ATOM | 1808 | HB | THR | 109 | −3.336 | 11.067 | 14.783 | 1.00 | 0.00 | 0.053 | 0.00 | 0.00 |
| ATOM | 1809 | OG1 | THR | 109 | −4.527 | 11.716 | 13.253 | 1.00 | 0.00 | −0.537 | 11.04 | −17.40 |
| ATOM | 1810 | HG1 | THR | 109 | −4.257 | 12.708 | 13.188 | 1.00 | 0.00 | 0.424 | 0.00 | 0.00 |
| ATOM | 1811 | CG2 | THR | 109 | −5.236 | 10.012 | 14.833 | 1.00 | 0.00 | −0.159 | 16.15 | 4.00 |
| ATOM | 1812 | HG2 | THR | 109 | −4.918 | 9.226 | 14.147 | 1.00 | 0.00 | 0.053 | 0.00 | 0.00 |
| ATOM | 1813 | HG2 | THR | 109 | −5.101 | 9.673 | 15.860 | 1.00 | 0.00 | 0.053 | 0.00 | 0.00 |
| ATOM | 1814 | HG2 | THR | 109 | −6.287 | 10.241 | 14.660 | 1.00 | 0.00 | 0.053 | 0.00 | 0.00 |
| ATOM | 1815 | N | LYS | 110S | −3.532 | 14.136 | 16.468 | 1.00 | 0.00 | −0.650 | 9.00 | −17.40 |
| ATOM | 1816 | HN | LYS | 110S | −3.886 | 13.814 | 17.380 | 1.00 | 0.00 | 0.440 | 0.00 | 0.00 |
| ATOM | 1817 | CA | LYS | 110S | −2.551 | 15.181 | 16.436 | 1.00 | 0.00 | 0.158 | 9.40 | 4.00 |
| ATOM | 1818 | HA | LYS | 110S | −2.855 | 16.011 | 15.799 | 1.00 | 0.00 | 0.053 | 0.00 | 0.00 |
| ATOM | 1819 | C | LYS | 110S | −1.419 | 14.408 | 15.896 | 1.00 | 0.00 | 0.396 | 9.82 | 4.00 |
| ATOM | 1820 | O | LYS | 110S | −0.591 | 14.887 | 15.124 | 1.00 | 0.00 | −0.396 | 8.17 | −17.40 |
| ATOM | 1821 | CB | LYS | 110S | −2.130 | 15.675 | 17.832 | 1.00 | 0.00 | −0.106 | 12.77 | 4.00 |
| ATOM | 1822 | HB1 | LYS | 110S | −1.125 | 16.091 | 17.757 | 1.00 | 0.00 | 0.053 | 0.00 | 0.00 |
| ATOM | 1823 | HB2 | LYS | 110S | −2.145 | 14.824 | 18.513 | 1.00 | 0.00 | 0.053 | 0.00 | 0.00 |
| ATOM | 1824 | CG | LYS | 110S | −3.033 | 16.756 | 18.425 | 1.00 | 0.00 | −0.106 | 12.77 | 4.00 |
| ATOM | 1825 | HG1 | LYS | 110S | −3.368 | 16.539 | 19.439 | 1.00 | 0.00 | 0.053 | 0.00 | 0.00 |
| ATOM | 1826 | HG2 | LYS | 110S | −3.947 | 16.923 | 17.856 | 1.00 | 0.00 | 0.053 | 0.00 | 0.00 |
| ATOM | 1827 | CD | LYS | 110S | −2.369 | 18.130 | 18.516 | 1.00 | 0.00 | −0.106 | 12.77 | 4.00 |
| ATOM | 1828 | HD1 | LYS | 110S | −3.052 | 18.930 | 18.800 | 1.00 | 0.00 | 0.053 | 0.00 | 0.00 |
| ATOM | 1829 | HD2 | LYS | 110S | −1.920 | 18.464 | 17.580 | 1.00 | 0.00 | 0.053 | 0.00 | 0.00 |
| ATOM | 1830 | CE | LYS | 110S | −1.237 | 18.201 | 19.539 | 1.00 | 0.00 | 0.099 | 12.77 | 4.00 |
| ATOM | 1831 | HE1 | LYS | 110S | −0.454 | 17.487 | 19.278 | 1.00 | 0.00 | 0.053 | 0.00 | 0.00 |
| ATOM | 1832 | HE2 | LYS | 110S | −1.617 | 17.962 | 20.532 | 1.00 | 0.00 | 0.053 | 0.00 | 0.00 |
| ATOM | 1833 | NZ | LYS | 110S | −0.658 | 19.565 | 19.559 | 1.00 | 0.00 | −0.045 | 13.25 | −39.20 |
| ATOM | 1834 | HZ1 | LYS | 110S | 0.104 | 19.604 | 20.250 | 1.00 | 0.00 | 0.280 | 0.00 | 0.00 |
| ATOM | 1835 | HZ2 | LYS | 110S | −0.286 | 19.794 | 18.626 | 1.00 | 0.00 | 0.280 | 0.00 | 0.00 |
| ATOM | 1836 | HZ3 | LYS | 110S | −1.388 | 20.244 | 19.814 | 1.00 | 0.00 | 0.280 | 0.00 | 0.00 |
| ATOM | 1837 | N | SER | 111 | −1.410 | 13.136 | 16.338 | 1.00 | 0.00 | −0.650 | 9.00 | −17.40 |
| ATOM | 1838 | HN | SER | 111 | −2.053 | 12.900 | 17.106 | 1.00 | 0.00 | 0.440 | 0.00 | 0.00 |
| ATOM | 1839 | CA | SER | 111 | −0.588 | 12.089 | 15.835 | 1.00 | 0.00 | 0.158 | 9.40 | 4.00 |
| ATOM | 1840 | HA | SER | 111 | 0.450 | 12.409 | 15.749 | 1.00 | 0.00 | 0.053 | 0.00 | 0.00 |
| ATOM | 1841 | C | SER | 111 | −1.199 | 11.803 | 14.499 | 1.00 | 0.00 | 0.396 | 9.82 | 4.00 |
| ATOM | 1842 | O | SER | 111 | −1.725 | 10.724 | 14.243 | 1.00 | 0.00 | −0.396 | 8.17 | −17.40 |
| ATOM | 1843 | CB | SER | 111 | −0.721 | 10.833 | 16.718 | 1.00 | 0.00 | 0.007 | 12.77 | 4.00 |
| ATOM | 1844 | HB1 | SER | 111 | −1.750 | 10.477 | 16.746 | 1.00 | 0.00 | 0.053 | 0.00 | 0.00 |
| ATOM | 1845 | HB2 | SER | 111 | −0.413 | 11.036 | 17.743 | 1.00 | 0.00 | 0.053 | 0.00 | 0.00 |
| ATOM | 1846 | OG | SER | 111 | 0.085 | 9.775 | 16.231 | 1.00 | 0.00 | −0.537 | 11.04 | 17.40 |
| ATOM | 1847 | HG | SER | 111 | −0.444 | 9.232 | 15.534 | 1.00 | 0.00 | 0.424 | 0.00 | 0.00 |
| ATOM | 1848 | N | GLU | 112 | −1.113 | 12.810 | 13.613 | 1.00 | 0.00 | −0.650 | 9.00 | −17.40 |
| ATOM | 1849 | HN | GLU | 112 | −0.551 | 13.623 | 13.903 | 1.00 | 0.00 | 0.440 | 0.00 | 0.00 |
| ATOM | 1850 | CA | GLU | 112 | −1.707 | 12.887 | 12.316 | 1.00 | 0.00 | 0.158 | 9.40 | 4.00 |
| ATOM | 1851 | HA | GLU | 112 | −2.787 | 12.770 | 12.397 | 1.00 | 0.00 | 0.053 | 0.00 | 0.00 |
| ATOM | 1852 | C | GLU | 112 | −1.164 | 11.809 | 11.449 | 1.00 | 0.00 | 0.396 | 9.82 | 4.00 |
| ATOM | 1853 | O | GLU | 112 | −0.452 | 10.910 | 11.892 | 1.00 | 0.00 | −0.396 | 8.17 | −17.40 |
| ATOM | 1854 | CB | GLU | 112 | −1.395 | 14.235 | 11.648 | 1.00 | 0.00 | −0.106 | 12.77 | 4.00 |
| ATOM | 1855 | HB1 | GLU | 112 | −2.077 | 15.016 | 11.982 | 1.00 | 0.00 | 0.053 | 0.00 | 0.00 |
| ATOM | 1856 | HB2 | GLU | 112 | −1.479 | 14.174 | 10.563 | 1.00 | 0.00 | 0.053 | 0.00 | 0.00 |
| ATOM | 1857 | CG | GLU | 112 | 0.026 | 14.706 | 11.967 | 1.00 | 0.00 | −0.106 | 12.77 | 4.00 |
| ATOM | 1858 | HG1 | GLU | 112 | 0.437 | 15.201 | 11.087 | 1.00 | 0.00 | 0.053 | 0.00 | 0.00 |
| ATOM | 1859 | HG2 | GLU | 112 | 0.632 | 13.838 | 12.227 | 1.00 | 0.00 | 0.053 | 0.00 | 0.00 |
| ATOM | 1860 | CD | GLU | 112 | −0.031 | 15.675 | 13.135 | 1.00 | 0.00 | 0.399 | 9.82 | 4.00 |
| ATOM | 1861 | OE1 | GLU | 112 | 1.053 | 16.011 | 13.679 | 1.00 | 0.00 | −0.396 | 8.17 | −18.95 |
| ATOM | 1862 | OE2 | GLU | 112 | −1.162 | 16.100 | 13.493 | 1.00 | 0.00 | −0.427 | 8.17 | −18.95 |
| ATOM | 1864 | N | ASP | 113P | −1.547 | 11.885 | 10.165 | 1.00 | 0.00 | −0.650 | 9.00 | −17.40 |
| ATOM | 1865 | HN | ASP | 113P | −2.144 | 12.675 | 9.885 | 1.00 | 0.00 | 0.440 | 0.00 | 0.00 |
| ATOM | 1866 | CA | ASP | 113P | −1.177 | 10.937 | 9.162 | 1.00 | 0.00 | 0.158 | 9.40 | 4.00 |
| ATOM | 1867 | HA | ASP | 113P | −1.429 | 9.935 | 9.508 | 1.00 | 0.00 | 0.053 | 0.00 | 0.00 |
| ATOM | 1868 | C | ASP | 113P | 0.291 | 11.034 | 8.918 | 1.00 | 0.00 | 0.396 | 9.82 | 4.00 |
| ATOM | 1869 | O | ASP | 113P | 0.904 | 10.055 | 8.497 | 1.00 | 0.00 | −0.396 | 8.17 | −17.40 |
| ATOM | 1870 | CB | ASP | 113P | −1.799 | 11.212 | 7.785 | 1.00 | 0.00 | −0.336 | 12.77 | 4.00 |
| ATOM | 1871 | HB1 | ASP | 113P | −2.870 | 11.346 | 7.932 | 1.00 | 0.00 | 0.053 | 0.00 | 0.00 |
| ATOM | 1872 | HB2 | ASP | 113P | −1.588 | 10.348 | 7.154 | 1.00 | 0.00 | 0.053 | 0.00 | 0.00 |
| ATOM | 1873 | CG | ASP | 113P | −1.148 | 12.478 | 7.235 | 1.00 | 0.00 | 0.297 | 9.82 | 4.00 |
| ATOM | 1874 | OD1 | ASP | 113P | −1.672 | 13.592 | 7.498 | 1.00 | 0.00 | −0.534 | 8.17 | −18.95 |
| ATOM | 1875 | OD2 | ASP | 113P | −0.106 | 12.341 | 6.540 | 1.00 | 0.00 | −0.534 | 8.17 | −18.95 |
| ATOM | 1876 | N | SER | 114 | 0.880 | 12.219 | 9.189 | 1.00 | 0.00 | −0.650 | 9.00 | −17.40 |
| ATOM | 1877 | HN | SER | 114 | 0.362 | 12.915 | 9.744 | 1.00 | 0.00 | 0.440 | 0.00 | 0.00 |
| ATOM | 1878 | CA | SER | 114 | 2.203 | 12.542 | 8.730 | 1.00 | 0.00 | 0.158 | 9.40 | 4.00 |
| ATOM | 1879 | HA | SER | 114 | 2.237 | 12.702 | 7.652 | 1.00 | 0.00 | 0.053 | 0.00 | 0.00 |
| ATOM | 1880 | C | SER | 114 | 3.181 | 11.464 | 9.030 | 1.00 | 0.00 | 0.396 | 9.82 | 4.00 |
| ATOM | 1881 | O | SER | 114 | 3.736 | 10.864 | 8.114 | 1.00 | 0.00 | −0.396 | 8.17 | −17.40 |
| ATOM | 1882 | CB | SER | 114 | 2.751 | 13.837 | 9.355 | 1.00 | 0.00 | 0.007 | 12.77 | 4.00 |
| ATOM | 1883 | HB1 | SER | 114 | 2.793 | 13.739 | 10.439 | 1.00 | 0.00 | 0.053 | 0.00 | 0.00 |
| ATOM | 1884 | HB2 | SER | 114 | 2.104 | 14.677 | 9.101 | 1.00 | 0.00 | 0.053 | 0.00 | 0.00 |
| ATOM | 1885 | OG | SER | 114 | 4.059 | 14.099 | 8.865 | 1.00 | 0.00 | −0.537 | 11.04 | −17.40 |
| ATOM | 1886 | HG | SER | 114 | 4.554 | 14.718 | 9.521 | 1.00 | 0.00 | 0.424 | 0.00 | 0.00 |
| ATOM | 1887 | N | TYR | 115 | 3.436 | 11.131 | 10.297 | 1.00 | 0.00 | −0.650 | 9.00 | −17.40 |
| ATOM | 1888 | HN | TYR | 115 | 3.009 | 11.590 | 11.114 | 1.00 | 0.00 | 0.440 | 0.00 | 0.00 |
| ATOM | 1889 | CA | TYR | 115 | 4.373 | 10.058 | 10.377 | 1.00 | 0.00 | 0.158 | 9.40 | 4.00 |
| ATOM | 1890 | HA | TYR | 115 | 4.230 | 9.485 | 9.460 | 1.00 | 0.00 | 0.053 | 0.00 | 0.00 |
| ATOM | 1891 | C | TYR | 115 | 4.004 | 9.313 | 11.597 | 1.00 | 0.00 | 0.396 | 9.82 | 4.00 |
| ATOM | 1892 | O | TYR | 115 | 4.868 | 8.898 | 12.363 | 1.00 | 0.00 | −0.396 | 8.17 | −17.40 |
| ATOM | 1893 | CB | TYR | 115 | 5.821 | 10.566 | 10.525 | 1.00 | 0.00 | −0.106 | 12.77 | 4.00 |
| ATOM | 1894 | HB1 | TYR | 115 | 5.957 | 10.992 | 11.518 | 1.00 | 0.00 | 0.053 | 0.00 | 0.00 |
| ATOM | 1895 | HB2 | TYR | 115 | 6.017 | 11.329 | 9.772 | 1.00 | 0.00 | 0.053 | 0.00 | 0.00 |
| ATOM | 1896 | CG | TYR | 115 | 6.769 | 9.429 | 10.338 | 1.00 | 0.00 | 0.000 | 7.26 | 0.60 |
| ATOM | 1897 | CD1 | TYR | 115 | 7.009 | 8.927 | 9.078 | 1.00 | 0.00 | −0.127 | 10.80 | 0.60 |
| ATOM | 1898 | HD1 | TYR | 115 | 6.498 | 9.360 | 8.218 | 1.00 | 0.00 | 0.127 | 0.00 | 0.00 |
| ATOM | 1899 | CD2 | TYR | 115 | 7.436 | 8.880 | 11.408 | 1.00 | 0.00 | −0.127 | 10.80 | 0.60 |
| ATOM | 1900 | HD2 | TYR | 115 | 7.265 | 9.275 | 12.409 | 1.00 | 0.00 | 0.127 | 0.00 | 0.00 |
| ATOM | 1901 | CE1 | TYR | 115 | 7.886 | 7.885 | 8.893 | 1.00 | 0.00 | −0.127 | 10.80 | 0.60 |
| ATOM | 1902 | HE1 | TYR | 115 | 8.063 | 7.492 | 7.891 | 1.00 | 0.00 | 0.127 | 0.00 | 0.00 |
| ATOM | 1903 | CE2 | TYR | 115 | 8.315 | 7.839 | 11.231 | 1.00 | 0.00 | −0.127 | 10.80 | 0.60 |
| ATOM | 1904 | HE2 | TYR | 115 | 8.833 | 7.410 | 12.088 | 1.00 | 0.00 | 0.127 | 0.00 | 0.00 |
| ATOM | 1905 | CZ | TYR | 115 | 8.539 | 7.339 | 9.971 | 1.00 | 0.00 | 0.026 | 7.26 | 0.60 |
| ATOM | 1906 | OH | TYR | 115 | 9.442 | 6.270 | 9.787 | 1.00 | 0.00 | −0.451 | 10.94 | −17.40 |
| ATOM | 1907 | HH | TYR | 115 | 8.926 | 5.434 | 9.476 | 1.00 | 0.00 | 0.424 | 0.00 | 0.00 |
| ATOM | 1908 | N | LEU | 116 | 2.695 | 9.086 | 11.801 | 1.00 | 0.00 | −0.650 | 9.00 | −17.40 |
| ATOM | 1909 | HN | LEU | 116 | 1.969 | 9.351 | 11.119 | 1.00 | 0.00 | 0.440 | 0.00 | 0.00 |
| ATOM | 1910 | CA | LEU | 116 | 2.395 | 8.449 | 13.036 | 1.00 | 0.00 | 0.158 | 9.40 | 4.00 |
| ATOM | 1911 | HA | LEU | 116 | 3.325 | 8.115 | 13.496 | 1.00 | 0.00 | 0.053 | 0.00 | 0.00 |
| ATOM | 1912 | C | LEU | 116 | 1.512 | 7.287 | 12.775 | 1.00 | 0.00 | 0.396 | 9.82 | 4.00 |
| ATOM | 1913 | O | LEU | 116 | 0.478 | 7.398 | 12.118 | 1.00 | 0.00 | −0.396 | 8.17 | −17.40 |
| ATOM | 1914 | CB | LEU | 116 | 1.710 | 9.392 | 14.022 | 1.00 | 0.00 | −0.106 | 12.77 | 4.00 |
| ATOM | 1915 | HB1 | LEU | 116 | 1.631 | 8.941 | 15.011 | 1.00 | 0.00 | 0.053 | 0.00 | 0.00 |
| ATOM | 1916 | HB2 | LEU | 116 | 0.701 | 9.643 | 13.693 | 1.00 | 0.00 | 0.053 | 0.00 | 0.00 |
| ATOM | 1917 | CG | LEU | 116 | 2.493 | 10.709 | 14.172 | 1.00 | 0.00 | −0.053 | 9.40 | 4.00 |
| ATOM | 1918 | HG | LEU | 116 | 3.562 | 10.509 | 14.241 | 1.00 | 0.00 | 0.053 | 0.00 | 0.00 |
| ATOM | 1919 | CD1 | LEU | 116 | 2.244 | 11.622 | 12.960 | 1.00 | 0.00 | −0.159 | 16.15 | 4.00 |
| ATOM | 1920 | HD1 | LEU | 116 | 2.804 | 12.548 | 13.081 | 1.00 | 0.00 | 0.053 | 0.00 | 0.00 |
| ATOM | 1921 | HD1 | LEU | 116 | 2.570 | 11.117 | 12.050 | 1.00 | 0.00 | 0.053 | 0.00 | 0.00 |
| ATOM | 1922 | HD1 | LEU | 116 | 1.180 | 11.848 | 12.887 | 1.00 | 0.00 | 0.053 | 0.00 | 0.00 |
| ATOM | 1923 | CD2 | LEU | 116 | 2.258 | 11.389 | 15.527 | 1.00 | 0.00 | −0.159 | 16.15 | 4.00 |
| ATOM | 1924 | HD2 | LEU | 116 | 2.835 | 12.312 | 15.575 | 1.00 | 0.00 | 0.053 | 0.00 | 0.00 |
| ATOM | 1925 | HD2 | LEU | 116 | 1.198 | 11.617 | 15.640 | 1.00 | 0.00 | 0.053 | 0.00 | 0.00 |
| ATOM | 1926 | HD2 | LEU | 116 | 2.573 | 10.721 | 16.328 | 1.00 | 0.00 | 0.053 | 0.00 | 0.00 |
| ATOM | 1927 | N | ALA | 117 | 1.937 | 6.120 | 13.284 | 1.00 | 0.00 | −0.650 | 9.00 | −17.40 |
| ATOM | 1928 | HN | ALA | 117 | 2.838 | 6.082 | 13.781 | 1.00 | 0.00 | 0.440 | 0.00 | 0.00 |
| ATOM | 1929 | CA | ALA | 117 | 1.162 | 4.932 | 13.148 | 1.00 | 0.00 | 0.158 | 9.40 | 4.00 |
| ATOM | 1930 | HA | ALA | 117 | 0.347 | 5.187 | 12.469 | 1.00 | 0.00 | 0.053 | 0.00 | 0.00 |
| ATOM | 1931 | C | ALA | 117 | 0.692 | 4.599 | 14.520 | 1.00 | 0.00 | 0.396 | 9.82 | 4.00 |
| ATOM | 1932 | O | ALA | 117 | 1.474 | 4.563 | 15.467 | 1.00 | 0.00 | −0.396 | 8.17 | −17.40 |
| ATOM | 1933 | CB | ALA | 117 | 1.960 | 3.725 | 12.624 | 1.00 | 0.00 | −0.159 | 16.15 | 4.00 |
| ATOM | 1934 | HB1 | ALA | 117 | 1.302 | 2.859 | 12.547 | 1.00 | 0.00 | 0.053 | 0.00 | 0.00 |
| ATOM | 1935 | HB2 | ALA | 117 | 2.368 | 3.958 | 11.640 | 1.00 | 0.00 | 0.053 | 0.00 | 0.00 |
| ATOM | 1936 | HB3 | ALA | 117 | 2.775 | 3.501 | 13.312 | 1.00 | 0.00 | 0.053 | 0.00 | 0.00 |
| ATOM | 1937 | N | ALA | 118 | −0.617 | 4.349 | 14.659 | 1.00 | 0.00 | −0.650 | 9.00 | −17.40 |
| ATOM | 1938 | HN | ALA | 118 | −1.228 | 4.361 | 13.830 | 1.00 | 0.00 | 0.440 | 0.00 | 0.00 |
| ATOM | 1939 | CA | ALA | 118 | −1.173 | 4.066 | 15.944 | 1.00 | 0.00 | 0.158 | 9.40 | 4.00 |
| ATOM | 1940 | HA | ALA | 118 | −0.860 | 4.845 | 16.639 | 1.00 | 0.00 | 0.053 | 0.00 | 0.00 |
| ATOM | 1941 | C | ALA | 118 | −0.672 | 2.736 | 16.400 | 1.00 | 0.00 | 0.396 | 9.82 | 4.00 |
| ATOM | 1942 | O | ALA | 118 | −0.304 | 1.882 | 15.594 | 1.00 | 0.00 | −0.396 | 8.17 | −17.40 |
| ATOM | 1943 | CB | ALA | 118 | −2.707 | 4.013 | 15.928 | 1.00 | 0.00 | −0.159 | 16.15 | 4.00 |
| ATOM | 1944 | HB1 | ALA | 118 | −3.074 | 3.794 | 16.930 | 1.00 | 0.00 | 0.053 | 0.00 | 0.00 |
| ATOM | 1945 | HB2 | ALA | 118 | −3.100 | 4.974 | 15.598 | 1.00 | 0.00 | 0.053 | 0.00 | 0.00 |
| ATOM | 1946 | HB3 | ALA | 118 | −3.037 | 3.232 | 15.242 | 1.00 | 0.00 | 0.053 | 0.00 | 0.00 |
| ATOM | 1947 | N | VAL | 119 | −0.627 | 2.558 | 17.735 | 1.00 | 0.00 | −0.650 | 9.00 | −17.40 |
| ATOM | 1948 | HN | VAL | 119 | −0.912 | 3.342 | 18.338 | 1.00 | 0.00 | 0.440 | 0.00 | 0.00 |
| ATOM | 1949 | CA | VAL | 119 | −0.206 | 1.342 | 18.364 | 1.00 | 0.00 | 0.158 | 9.40 | 4.00 |
| ATOM | 1950 | HA | VAL | 119 | 0.737 | 1.063 | 17.893 | 1.00 | 0.00 | 0.053 | 0.00 | 0.00 |
| ATOM | 1951 | C | VAL | 119 | −1.291 | 0.337 | 18.109 | 1.00 | 0.00 | 0.396 | 9.82 | 4.00 |
| ATOM | 1952 | O | VAL | 119 | −2.392 | 0.692 | 17.690 | 1.00 | 0.00 | −0.396 | 8.17 | −17.40 |
| ATOM | 1953 | CB | VAL | 119 | −0.034 | 1.522 | 19.856 | 1.00 | 0.00 | −0.053 | 9.40 | 4.00 |
| ATOM | 1954 | HB | VAL | 119 | −0.967 | 1.848 | 20.314 | 1.00 | 0.00 | 0.053 | 0.00 | 0.00 |
| ATOM | 1955 | CG1 | VAL | 119 | 0.391 | 0.197 | 20.518 | 1.00 | 0.00 | −0.159 | 16.15 | 4.00 |
| ATOM | 1956 | HG1 | VAL | 119 | 0.509 | 0.347 | 21.591 | 1.00 | 0.00 | 0.053 | 0.00 | 0.00 |
| ATOM | 1957 | HG1 | VAL | 119 | −0.372 | −0.560 | 20.340 | 1.00 | 0.00 | 0.053 | 0.00 | 0.00 |
| ATOM | 1958 | HG1 | VAL | 119 | 1.337 | −0.135 | 20.091 | 1.00 | 0.00 | 0.053 | 0.00 | 0.00 |
| ATOM | 1959 | CG2 | VAL | 119 | 0.967 | 2.668 | 20.078 | 1.00 | 0.00 | −0.159 | 16.15 | 4.00 |
| ATOM | 1960 | HG2 | VAL | 119 | 1.112 | 2.822 | 21.147 | 1.00 | 0.00 | 0.053 | 0.00 | 0.00 |
| ATOM | 1961 | HG2 | VAL | 119 | 1.920 | 2.413 | 19.615 | 1.00 | 0.00 | 0.053 | 0.00 | 0.00 |
| ATOM | 1962 | HG2 | VAL | 119 | 0.578 | 3.582 | 19.629 | 1.00 | 0.00 | 0.053 | 0.00 | 0.00 |
| ATOM | 1963 | N | MET | 120 | −0.990 | −0.954 | 18.353 | 1.00 | 0.00 | −0.650 | 9.00 | −17.40 |
| ATOM | 1964 | HN | MET | 120 | −0.060 | −1.181 | 18.734 | 1.00 | 0.00 | 0.440 | 0.00 | 0.00 |
| ATOM | 1965 | CA | MET | 120 | −1.912 | −2.024 | 18.103 | 1.00 | 0.00 | 0.158 | 9.40 | 4.00 |
| ATOM | 1966 | HA | MET | 120 | −2.158 | −1.997 | 17.041 | 1.00 | 0.00 | 0.053 | 0.00 | 0.00 |
| ATOM | 1967 | C | MET | 120 | −3.115 | −1.780 | 18.952 | 1.00 | 0.00 | 0.396 | 9.82 | 4.00 |
| ATOM | 1968 | O | MET | 120 | −4.250 | −1.968 | 18.520 | 1.00 | 0.00 | −0.396 | 8.17 | −17.40 |
| ATOM | 1969 | CB | MET | 120 | −1.390 | −3.377 | 18.604 | 1.00 | 0.00 | −0.106 | 12.77 | 4.00 |
| ATOM | 1970 | HB1 | MET | 120 | −2.110 | −4.142 | 18.317 | 1.00 | 0.00 | 0.053 | 0.00 | 0.00 |
| ATOM | 1971 | HB2 | MET | 120 | −1.293 | −3.320 | 19.688 | 1.00 | 0.00 | 0.053 | 0.00 | 0.00 |
| ATOM | 1972 | CG | MET | 120 | −0.034 | −3.804 | 18.052 | 1.00 | 0.00 | −0.041 | 12.77 | 4.00 |
| ATOM | 1973 | HG1 | MET | 120 | 0.721 | −3.023 | 18.142 | 1.00 | 0.00 | 0.053 | 0.00 | 0.00 |
| ATOM | 1974 | HG2 | MET | 120 | −0.074 | −4.064 | 16.994 | 1.00 | 0.00 | 0.053 | 0.00 | 0.00 |
| ATOM | 1975 | SD | MET | 120 | 0.641 | −5.258 | 18.903 | 1.00 | 0.00 | −0.130 | 16.39 | −6.40 |
| ATOM | 1976 | CE | MET | 120 | 0.460 | −4.504 | 20.545 | 1.00 | 0.00 | −0.094 | 16.15 | 4.00 |
| ATOM | 1977 | HE1 | MET | 120 | 0.812 | −5.200 | 21.306 | 1.00 | 0.00 | 0.053 | 0.00 | 0.00 |
| ATOM | 1978 | HE2 | MET | 120 | 1.048 | −3.587 | 20.591 | 1.00 | 0.00 | 0.053 | 0.00 | 0.00 |
| ATOM | 1979 | HE3 | MET | 120 | −0.589 | −4.270 | 20.724 | 1.00 | 0.00 | 0.053 | 0.00 | 0.00 |
| ATOM | 1980 | N | GLY | 121 | −2.851 | −1.368 | 20.203 | 1.00 | 0.00 | −0.650 | 9.00 | −17.40 |
| ATOM | 1981 | HN | GLY | 121 | −1.860 | −1.185 | 20.418 | 1.00 | 0.00 | 0.440 | 0.00 | 0.00 |
| ATOM | 1982 | CA | GLY | 121 | −3.779 | −1.151 | 21.276 | 1.00 | 0.00 | 0.105 | 9.40 | 4.00 |
| ATOM | 1983 | HA1 | GLY | 121 | −3.300 | −1.068 | 22.251 | 1.00 | 0.00 | 0.053 | 0.00 | 0.00 |
| ATOM | 1984 | HA2 | GLY | 121 | −4.512 | −1.950 | 21.381 | 1.00 | 0.00 | 0.053 | 0.00 | 0.00 |
| ATOM | 1985 | C | GLY | 121 | −4.584 | 0.107 | 21.122 | 1.00 | 0.00 | 0.396 | 9.82 | 4.00 |
| ATOM | 1986 | O | GLY | 121 | −5.389 | 0.404 | 22.000 | 1.00 | 0.00 | −0.396 | 8.17 | −17.40 |
| ATOM | 1987 | N | ILE | 122 | −4.368 | 0.920 | 20.070 | 1.00 | 0.00 | −0.650 | 9.00 | −17.40 |
| ATOM | 1988 | HN | ILE | 122 | −3.757 | 0.623 | 19.295 | 1.00 | 0.00 | 0.440 | 0.00 | 0.00 |
| ATOM | 1989 | CA | ILE | 122 | −5.006 | 2.210 | 20.048 | 1.00 | 0.00 | 0.158 | 9.40 | 4.00 |
| ATOM | 1990 | HA | ILE | 122 | −4.674 | 2.858 | 20.859 | 1.00 | 0.00 | 0.053 | 0.00 | 0.00 |
| ATOM | 1991 | C | ILE | 122 | −6.499 | 2.122 | 20.173 | 1.00 | 0.00 | 0.396 | 9.82 | 4.00 |
| ATOM | 1992 | O | ILE | 122 | −7.082 | 2.856 | 20.966 | 1.00 | 0.00 | −0.396 | 8.17 | −17.40 |
| ATOM | 1993 | CB | ILE | 122 | −4.727 | 3.007 | 18.807 | 1.00 | 0.00 | −0.053 | 9.40 | 4.00 |
| ATOM | 1994 | HB | ILE | 122 | −5.219 | 3.971 | 18.930 | 1.00 | 0.00 | 0.053 | 0.00 | 0.00 |
| ATOM | 1995 | CG1 | ILE | 122 | −5.384 | 2.391 | 17.558 | 1.00 | 0.00 | −0.106 | 12.77 | 4.00 |
| ATOM | 1996 | HG1 | ILE | 122 | −5.015 | 2.928 | 16.684 | 1.00 | 0.00 | 0.053 | 0.00 | 0.00 |
| ATOM | 1997 | HG1 | ILE | 122 | −6.463 | 2.501 | 17.656 | 1.00 | 0.00 | 0.053 | 0.00 | 0.00 |
| ATOM | 1998 | CG2 | ILE | 122 | −3.206 | 3.153 | 18.701 | 1.00 | 0.00 | −0.159 | 16.15 | 4.00 |
| ATOM | 1999 | HG2 | ILE | 122 | −2.957 | 3.728 | 17.809 | 1.00 | 0.00 | 0.053 | 0.00 | 0.00 |
| ATOM | 2000 | HG2 | ILE | 122 | −2.827 | 3.669 | 19.583 | 1.00 | 0.00 | 0.053 | 0.00 | 0.00 |
| ATOM | 2001 | HG2 | ILE | 122 | −2.749 | 2.165 | 18.635 | 1.00 | 0.00 | 0.053 | 0.00 | 0.00 |
| ATOM | 2002 | CD1 | ILE | 122 | −5.101 | 0.907 | 17.328 | 1.00 | 0.00 | −0.159 | 16.15 | 4.00 |
| ATOM | 2003 | HD1 | ILE | 122 | −5.610 | 0.575 | 16.423 | 1.00 | 0.00 | 0.053 | 0.00 | 0.00 |
| ATOM | 2004 | HD1 | ILE | 122 | −4.027 | 0.754 | 17.216 | 1.00 | 0.00 | 0.053 | 0.00 | 0.00 |
| ATOM | 2005 | HD1 | ILE | 122 | −5.462 | 0.331 | 18.180 | 1.00 | 0.00 | 0.053 | 0.00 | 0.00 |
| ATOM | 2006 | N | ALA | 123 | −7.180 | 1.251 | 19.409 | 1.00 | 0.00 | −0.650 | 9.00 | −17.40 |
| ATOM | 2007 | HN | ALA | 123 | −6.681 | 0.618 | 18.767 | 1.00 | 0.00 | 0.440 | 0.00 | 0.00 |
| ATOM | 2008 | CA | ALA | 123 | −8.617 | 1.214 | 19.500 | 1.00 | 0.00 | 0.158 | 9.40 | 4.00 |
| ATOM | 2009 | HA | ALA | 123 | −9.028 | 2.213 | 19.362 | 1.00 | 0.00 | 0.053 | 0.00 | 0.00 |
| ATOM | 2010 | C | ALA | 123 | −9.038 | 0.701 | 20.845 | 1.00 | 0.00 | 0.396 | 9.82 | 4.00 |
| ATOM | 2011 | O | ALA | 123 | −9.979 | 1.210 | 21.459 | 1.00 | 0.00 | −0.396 | 8.17 | −17.40 |
| ATOM | 2012 | CB | ALA | 123 | −9.262 | 0.319 | 18.426 | 1.00 | 0.00 | −0.159 | 16.15 | 4.00 |
| ATOM | 2013 | HB1 | ALA | 123 | −10.345 | 0.330 | 18.545 | 1.00 | 0.00 | 0.053 | 0.00 | 0.00 |
| ATOM | 2014 | HB2 | ALA | 123 | −9.001 | 0.693 | 17.436 | 1.00 | 0.00 | 0.053 | 0.00 | 0.00 |
| ATOM | 2015 | HB3 | ALA | 123 | −8.896 | −0.701 | 18.535 | 1.00 | 0.00 | 0.053 | 0.00 | 0.00 |
| ATOM | 2016 | N | SER | 124 | −8.334 | −0.327 | 21.346 | 1.00 | 0.00 | −0.650 | 9.00 | −17.40 |
| ATOM | 2017 | HN | SER | 124 | −7.518 | −0.690 | 20.832 | 1.00 | 0.00 | 0.440 | 0.00 | 0.00 |
| ATOM | 2018 | CA | SER | 124 | −8.716 | −0.919 | 22.592 | 1.00 | 0.00 | 0.158 | 9.40 | 4.00 |
| ATOM | 2019 | HA | SER | 124 | −9.739 | −1.280 | 22.487 | 1.00 | 0.00 | 0.053 | 0.00 | 0.00 |
| ATOM | 2020 | C | SER | 124 | −8.622 | 0.133 | 23.641 | 1.00 | 0.00 | 0.396 | 9.82 | 4.00 |
| ATOM | 2021 | O | SER | 124 | −9.501 | 0.246 | 24.496 | 1.00 | 0.00 | −0.396 | 8.17 | −17.40 |
| ATOM | 2022 | CB | SER | 124 | −7.798 | −2.078 | 23.015 | 1.00 | 0.00 | 0.007 | 12.77 | 4.00 |
| ATOM | 2023 | HB1 | SER | 124 | −8.089 | −2.453 | 23.995 | 1.00 | 0.00 | 0.053 | 0.00 | 0.00 |
| ATOM | 2024 | HB2 | SER | 124 | −6.762 | −1.741 | 23.065 | 1.00 | 0.00 | 0.053 | 0.00 | 0.00 |
| ATOM | 2025 | OG | SER | 124 | −7.884 | −3.142 | 22.079 | 1.00 | 0.00 | −0.537 | 11.04 | −17.40 |
| ATOM | 2026 | HG | SER | 124 | −8.632 | −2.942 | 21.400 | 1.00 | 0.00 | 0.424 | 0.00 | 0.00 |
| ATOM | 2027 | N | CYS | 125S | −7.563 | 0.961 | 23.583 | 1.00 | 0.00 | −0.65 | 9.00 | −17.40 |
| ATOM | 2028 | HN | CYS | 125S | −6.888 | 0.895 | 22.807 | 1.00 | 0.00 | 0.44 | 0.00 | 0.00 |
| ATOM | 2029 | CA | CYS | 125S | −7.390 | 1.933 | 24.616 | 1.00 | 0.00 | 0.15 | 9.40 | 4.00 |
| ATOM | 2030 | HA | CYS | 125S | −7.350 | 1.436 | 25.585 | 1.00 | 0.00 | 0.05 | 0.00 | 0.00 |
| ATOM | 2031 | C | CYS | 125S | −8.538 | 2.896 | 24.601 | 1.00 | 0.00 | 0.39 | 9.82 | 4.00 |
| ATOM | 2032 | O | CYS | 125S | −9.050 | 3.250 | 25.658 | 1.00 | 0.00 | −0.39 | 8.17 | −17.40 |
| ATOM | 2033 | CB | CYS | 125S | −6.086 | 2.757 | 24.520 | 1.00 | 0.00 | −0.04 | 12.77 | 4.00 |
| ATOM | 2034 | HB1 | CYS | 125S | −5.284 | 2.087 | 24.207 | 1.00 | 0.00 | 0.05 | 0.00 | 0.00 |
| ATOM | 2035 | HB2 | CYS | 125S | −5.877 | 3.177 | 25.503 | 1.00 | 0.00 | 0.05 | 0.00 | 0.00 |
| ATOM | 2036 | SG | CYS | 125S | −6.134 | 4.139 | 23.336 | 1.00 | 0.00 | −0.25 | 19.93 | −6.40 |
| ATOM | 2037 | HG | CYS | 125S | −5.186 | 5.032 | 23.652 | 1.00 | 0.00 | 0.19 | 0.00 | 0.00 |
| ATOM | 2038 | N | PHE | 126 | −8.983 | 3.350 | 23.411 | 1.00 | 0.00 | −0.650 | 9.00 | −17.40 |
| ATOM | 2039 | HN | PHE | 126 | −8.559 | 3.000 | 22.539 | 1.00 | 0.00 | 0.440 | 0.00 | 0.00 |
| ATOM | 2040 | CA | PHE | 126 | −10.046 | 4.320 | 23.350 | 1.00 | 0.00 | 0.158 | 9.40 | 4.00 |
| ATOM | 2041 | HA | PHE | 126 | −9.814 | 5.196 | 23.954 | 1.00 | 0.00 | 0.053 | 0.00 | 0.00 |
| ATOM | 2042 | C | PHE | 126 | −11.322 | 3.750 | 23.851 | 1.00 | 0.00 | 0.396 | 9.82 | 4.00 |
| ATOM | 2043 | O | PHE | 126 | −12.047 | 4.401 | 24.601 | 1.00 | 0.00 | −0.396 | 8.17 | −17.40 |
| ATOM | 2044 | CB | PHE | 126 | −10.438 | 4.780 | 21.935 | 1.00 | 0.00 | −0.106 | 12.77 | 4.00 |
| ATOM | 2045 | HB1 | PHE | 126 | −11.346 | 5.382 | 21.970 | 1.00 | 0.00 | 0.053 | 0.00 | 0.00 |
| ATOM | 2046 | HB2 | PHE | 126 | −10.617 | 3.918 | 21.292 | 1.00 | 0.00 | 0.053 | 0.00 | 0.00 |
| ATOM | 2047 | CG | PHE | 126 | −9.358 | 5.592 | 21.342 | 1.00 | 0.00 | 0.000 | 7.26 | 0.60 |
| ATOM | 2048 | CD1 | PHE | 126 | −9.133 | 6.869 | 21.793 | 1.00 | 0.00 | −0.127 | 10.80 | 0.60 |
| ATOM | 2049 | HD1 | PHE | 126 | −9.753 | 7.284 | 22.586 | 1.00 | 0.00 | 0.127 | 0.00 | 0.00 |
| ATOM | 2050 | CD2 | PHE | 126 | −8.571 | 5.067 | 20.349 | 1.00 | 0.00 | −0.127 | 10.80 | 0.60 |
| ATOM | 2051 | HD2 | PHE | 126 | −8.748 | 4.050 | 19.998 | 1.00 | 0.00 | 0.127 | 0.00 | 0.00 |
| ATOM | 2052 | CE1 | PHE | 126 | −8.127 | 7.621 | 21.244 | 1.00 | 0.00 | −0.127 | 10.80 | 0.60 |
| ATOM | 2053 | HE1 | PHE | 126 | −7.947 | 8.636 | 21.598 | 1.00 | 0.00 | 0.127 | 0.00 | 0.00 |
| ATOM | 2054 | CE2 | PHE | 126 | −7.564 | 5.812 | 19.795 | 1.00 | 0.00 | −0.127 | 10.80 | 0.60 |
| ATOM | 2055 | HE2 | PHE | 126 | −6.941 | 5.393 | 19.004 | 1.00 | 0.00 | 0.127 | 0.00 | 0.00 |
| ATOM | 2056 | CZ | PHE | 126 | −7.346 | 7.090 | 20.245 | 1.00 | 0.00 | −0.127 | 10.80 | 0.60 |
| ATOM | 2057 | HZ | PHE | 126 | −6.547 | 7.688 | 19.806 | 1.00 | 0.00 | 0.127 | 0.00 | 0.00 |
| ATOM | 2058 | N | SER | 127 | −11.644 | 2.517 | 23.434 | 1.00 | 0.00 | −0.650 | 9.00 | −17.40 |
| ATOM | 2059 | HN | SER | 127 | −10.975 | 1.966 | 22.877 | 1.00 | 0.00 | 0.440 | 0.00 | 0.00 |
| ATOM | 2060 | CA | SER | 127 | −12.922 | 1.973 | 23.768 | 1.00 | 0.00 | 0.158 | 9.40 | 4.00 |
| ATOM | 2061 | HA | SER | 127 | −13.719 | 2.612 | 23.389 | 1.00 | 0.00 | 0.053 | 0.00 | 0.00 |
| ATOM | 2062 | C | SER | 127 | −13.071 | 1.859 | 25.251 | 1.00 | 0.00 | 0.396 | 9.82 | 4.00 |
| ATOM | 2063 | O | SER | 127 | −14.112 | 2.216 | 25.798 | 1.00 | 0.00 | −0.396 | 8.17 | −17.40 |
| ATOM | 2064 | CB | SER | 127 | −13.156 | 0.581 | 23.156 | 1.00 | 0.00 | 0.007 | 12.77 | 4.00 |
| ATOM | 2065 | HB1 | SER | 127 | −14.050 | 0.130 | 23.586 | 1.00 | 0.00 | 0.053 | 0.00 | 0.00 |
| ATOM | 2066 | HB2 | SER | 127 | −12.301 | −0.063 | 23.360 | 1.00 | 0.00 | 0.053 | 0.00 | 0.00 |
| ATOM | 2067 | OG | SER | 127 | −13.326 | 0.692 | 21.750 | 1.00 | 0.00 | −0.537 | 11.04 | −17.40 |
| ATOM | 2068 | HG | SER | 127 | −13.291 | 1.684 | 21.476 | 1.00 | 0.00 | 0.424 | 0.00 | 0.00 |
| ATOM | 2069 | N | ASN | 128 | −12.034 | 1.366 | 25.951 | 1.00 | 0.00 | −0.650 | 9.00 | −17.40 |
| ATOM | 2070 | HN | ASN | 128 | −11.140 | 1.158 | 25.482 | 1.00 | 0.00 | 0.440 | 0.00 | 0.00 |
| ATOM | 2071 | CA | ASN | 128 | −12.186 | 1.132 | 27.361 | 1.00 | 0.00 | 0.158 | 9.40 | 4.00 |
| ATOM | 2072 | HA | ASN | 128 | −13.097 | 0.575 | 27.578 | 1.00 | 0.00 | 0.053 | 0.00 | 0.00 |
| ATOM | 2073 | C | ASN | 128 | −12.254 | 2.413 | 28.125 | 1.00 | 0.00 | 0.396 | 9.82 | 4.00 |
| ATOM | 2074 | O | ASN | 128 | −13.006 | 2.535 | 29.091 | 1.00 | 0.00 | −0.396 | 8.17 | −17.40 |
| ATOM | 2075 | CB | ASN | 128 | −11.021 | 0.344 | 27.974 | 1.00 | 0.00 | −0.106 | 12.77 | 4.00 |
| ATOM | 2076 | HB1 | ASN | 128 | −11.212 | 0.242 | 29.042 | 1.00 | 0.00 | 0.053 | 0.00 | 0.00 |
| ATOM | 2077 | HB2 | ASN | 128 | −10.105 | 0.906 | 27.791 | 1.00 | 0.00 | 0.053 | 0.00 | 0.00 |
| ATOM | 2078 | CG | ASN | 128 | −10.972 | −1.005 | 27.301 | 1.00 | 0.00 | 0.396 | 9.82 | 4.00 |
| ATOM | 2079 | OD1 | ASN | 128 | −11.928 | −1.780 | 27.317 | 1.00 | 0.00 | −0.396 | 8.17 | −17.40 |
| ATOM | 2080 | ND2 | ASN | 128 | −9.803 | −1.279 | 26.664 | 1.00 | 0.00 | −0.879 | 13.25 | −17.40 |
| ATOM | 2081 | HD2 | ASN | 128 | −9.033 | −0.594 | 26.682 | 1.00 | 0.00 | 0.440 | 0.00 | 0.00 |
| ATOM | 2082 | HD2 | ASN | 128 | −9.686 | −2.171 | 26.163 | 1.00 | 0.00 | 0.440 | 0.00 | 0.00 |
| ATOM | 2083 | N | VAL | 129 | −11.435 | 3.386 | 27.708 | 1.00 | 0.00 | −0.650 | 9.00 | −17.40 |
| ATOM | 2084 | HN | VAL | 129 | −10.920 | 3.243 | 26.827 | 1.00 | 0.00 | 0.440 | 0.00 | 0.00 |
| ATOM | 2085 | CA | VAL | 129 | −11.234 | 4.613 | 28.415 | 1.00 | 0.00 | 0.158 | 9.40 | 4.00 |
| ATOM | 2086 | HA | VAL | 129 | −11.128 | 4.475 | 29.491 | 1.00 | 0.00 | 0.053 | 0.00 | 0.00 |
| ATOM | 2087 | C | VAL | 129 | −12.367 | 5.588 | 28.242 | 1.00 | 0.00 | 0.396 | 9.82 | 4.00 |
| ATOM | 2088 | O | VAL | 129 | −12.507 | 6.479 | 29.076 | 1.00 | 0.00 | −0.396 | 8.17 | −17.40 |
| ATOM | 2089 | CB | VAL | 129 | −9.959 | 5.296 | 27.987 | 1.00 | 0.00 | −0.053 | 9.40 | 4.00 |
| ATOM | 2090 | HB | VAL | 129 | −10.003 | 5.511 | 26.919 | 1.00 | 0.00 | 0.053 | 0.00 | 0.00 |
| ATOM | 2091 | CG1 | VAL | 129 | −9.795 | 6.609 | 28.769 | 1.00 | 0.00 | −0.159 | 16.15 | 4.00 |
| ATOM | 2092 | HG1 | VAL | 129 | −8.873 | 7.103 | 28.460 | 1.00 | 0.00 | 0.053 | 0.00 | 0.00 |
| ATOM | 2093 | HG1 | VAL | 129 | −10.643 | 7.262 | 28.565 | 1.00 | 0.00 | 0.053 | 0.00 | 0.00 |
| ATOM | 2094 | HG1 | VAL | 129 | −9.751 | 6.393 | 29.836 | 1.00 | 0.00 | 0.053 | 0.00 | 0.00 |
| ATOM | 2095 | CG2 | VAL | 129 | −8.785 | 4.321 | 28.166 | 1.00 | 0.00 | −0.159 | 16.15 | 4.00 |
| ATOM | 2096 | HG2 | VAL | 129 | −7.858 | 4.806 | 27.858 | 1.00 | 0.00 | 0.053 | 0.00 | 0.00 |
| ATOM | 2097 | HG2 | VAL | 129 | −8.711 | 4.028 | 29.213 | 1.00 | 0.00 | 0.053 | 0.00 | 0.00 |
| ATOM | 2098 | HG2 | VAL | 129 | −8.950 | 3.435 | 27.552 | 1.00 | 0.00 | 0.053 | 0.00 | 0.00 |
| ATOM | 2099 | N | PHE | 130 | −13.199 | 5.469 | 27.177 | 1.00 | 0.00 | −0.650 | 9.00 | −17.40 |
| ATOM | 2100 | HN | PHE | 130 | −13.170 | 4.631 | 26.577 | 1.00 | 0.00 | 0.440 | 0.00 | 0.00 |
| ATOM | 2101 | CA | PHE | 130 | −14.121 | 6.536 | 26.903 | 1.00 | 0.00 | 0.158 | 9.40 | 4.00 |
| ATOM | 2102 | HA | PHE | 130 | −13.559 | 7.454 | 26.731 | 1.00 | 0.00 | 0.053 | 0.00 | 0.00 |
| ATOM | 2103 | C | PHE | 130 | −15.038 | 6.707 | 28.086 | 1.00 | 0.00 | 0.396 | 9.82 | 4.00 |
| ATOM | 2104 | O | PHE | 130 | −15.396 | 7.827 | 28.449 | 1.00 | 0.00 | −0.396 | 8.17 | −17.40 |
| ATOM | 2105 | CB | PHE | 130 | −14.976 | 6.363 | 25.630 | 1.00 | 0.00 | −0.106 | 12.77 | 4.00 |
| ATOM | 2106 | HB1 | PHE | 130 | −14.596 | 5.558 | 25.000 | 1.00 | 0.00 | 0.053 | 0.00 | 0.00 |
| ATOM | 2107 | HB2 | PHE | 130 | −14.983 | 7.271 | 25.027 | 1.00 | 0.00 | 0.053 | 0.00 | 0.00 |
| ATOM | 2108 | CG | PHE | 130 | −16.386 | 6.039 | 25.962 | 1.00 | 0.00 | 0.000 | 7.26 | 0.60 |
| ATOM | 2109 | CD1 | PHE | 130 | −17.241 | 7.107 | 26.136 | 1.00 | 0.00 | −0.127 | 10.80 | 0.60 |
| ATOM | 2110 | HD1 | PHE | 130 | −16.854 | 8.120 | 26.030 | 1.00 | 0.00 | 0.127 | 0.00 | 0.00 |
| ATOM | 2111 | CD2 | PHE | 130 | −16.869 | 4.759 | 26.091 | 1.00 | 0.00 | −0.127 | 10.80 | 0.60 |
| ATOM | 2112 | HD2 | PHE | 130 | −16.207 | 3.903 | 25.953 | 1.00 | 0.00 | 0.127 | 0.00 | 0.00 |
| ATOM | 2113 | CE1 | PHE | 130 | −18.566 | 6.923 | 26.439 | 1.00 | 0.00 | −0.127 | 10.80 | 0.60 |
| ATOM | 2114 | HE1 | PHE | 130 | −19.228 | 7.778 | 26.572 | 1.00 | 0.00 | 0.127 | 0.00 | 0.00 |
| ATOM | 2115 | CE2 | PHE | 130 | −18.201 | 4.569 | 26.396 | 1.00 | 0.00 | −0.127 | 10.80 | 0.60 |
| ATOM | 2116 | HE2 | PHE | 130 | −18.591 | 3.556 | 26.499 | 1.00 | 0.00 | 0.127 | 0.00 | 0.00 |
| ATOM | 2117 | CZ | PHE | 130 | −19.042 | 5.645 | 26.571 | 1.00 | 0.00 | −0.127 | 10.80 | 0.60 |
| ATOM | 2118 | HZ | PHE | 130 | −20.091 | 5.479 | 26.815 | 1.00 | 0.00 | 0.127 | 0.00 | 0.00 |
| ATOM | 2119 | N | VAL | 131 | −15.435 | 5.608 | 28.753 | 1.00 | 0.00 | −0.650 | 9.00 | −17.40 |
| ATOM | 2120 | HN | VAL | 131 | −15.111 | 4.672 | 28.469 | 1.00 | 0.00 | 0.440 | 0.00 | 0.00 |
| ATOM | 2121 | CA | VAL | 131 | −16.321 | 5.774 | 29.871 | 1.00 | 0.00 | 0.158 | 9.40 | 4.00 |
| ATOM | 2122 | HA | VAL | 131 | −17.213 | 6.313 | 29.555 | 1.00 | 0.00 | 0.053 | 0.00 | 0.00 |
| ATOM | 2123 | C | VAL | 131 | −15.629 | 6.552 | 30.954 | 1.00 | 0.00 | 0.396 | 9.82 | 4.00 |
| ATOM | 2124 | O | VAL | 131 | −16.237 | 7.415 | 31.587 | 1.00 | 0.00 | −0.396 | 8.17 | −17.40 |
| ATOM | 2125 | CB | VAL | 131 | −16.807 | 4.475 | 30.451 | 1.00 | 0.00 | −0.053 | 9.40 | 4.00 |
| ATOM | 2126 | HB | VAL | 131 | −17.440 | 4.698 | 31.309 | 1.00 | 0.00 | 0.053 | 0.00 | 0.00 |
| ATOM | 2127 | CG1 | VAL | 131 | −17.607 | 3.730 | 29.367 | 1.00 | 0.00 | −0.159 | 16.15 | 4.00 |
| ATOM | 2128 | HG1 | VAL | 131 | −17.969 | 2.783 | 29.768 | 1.00 | 0.00 | 0.053 | 0.00 | 0.00 |
| ATOM | 2129 | HG1 | VAL | 131 | −18.454 | 4.340 | 29.056 | 1.00 | 0.00 | 0.053 | 0.00 | 0.00 |
| ATOM | 2130 | HG1 | VAL | 131 | −16.964 | 3.537 | 28.507 | 1.00 | 0.00 | 0.053 | 0.00 | 0.00 |
| ATOM | 2131 | CG2 | VAL | 131 | −15.609 | 3.685 | 31.001 | 1.00 | 0.00 | −0.159 | 16.15 | 4.00 |
| ATOM | 2132 | HG2 | VAL | 131 | −15.957 | 2.742 | 31.422 | 1.00 | 0.00 | 0.053 | 0.00 | 0.00 |
| ATOM | 2133 | HG2 | VAL | 131 | −14.904 | 3.483 | 30.193 | 1.00 | 0.00 | 0.053 | 0.00 | 0.00 |
| ATOM | 2134 | HG2 | VAL | 131 | −15.113 | 4.268 | 31.777 | 1.00 | 0.00 | 0.053 | 0.00 | 0.00 |
| ATOM | 2135 | N | ALA | 132 | −14.332 | 6.278 | 31.190 | 1.00 | 0.00 | −0.650 | 9.00 | −17.40 |
| ATOM | 2136 | HN | ALA | 132 | −13.858 | 5.577 | 30.602 | 1.00 | 0.00 | 0.440 | 0.00 | 0.00 |
| ATOM | 2137 | CA | ALA | 132 | −13.582 | 6.928 | 32.234 | 1.00 | 0.00 | 0.158 | 9.40 | 4.00 |
| ATOM | 2138 | HA | ALA | 132 | −14.082 | 6.784 | 33.191 | 1.00 | 0.00 | 0.053 | 0.00 | 0.00 |
| ATOM | 2139 | C | ALA | 132 | −13.481 | 8.395 | 31.947 | 1.00 | 0.00 | 0.396 | 9.82 | 4.00 |
| ATOM | 2140 | O | ALA | 132 | −13.588 | 9.212 | 32.860 | 1.00 | 0.00 | −0.396 | 8.17 | −17.40 |
| ATOM | 2141 | CB | ALA | 132 | −12.147 | 6.383 | 32.356 | 1.00 | 0.00 | −0.159 | 16.15 | 4.00 |
| ATOM | 2142 | HB1 | ALA | 132 | −11.625 | 6.906 | 33.157 | 1.00 | 0.00 | 0.053 | 0.00 | 0.00 |
| ATOM | 2143 | HB2 | ALA | 132 | −12.180 | 5.317 | 32.581 | 1.00 | 0.00 | 0.053 | 0.00 | 0.00 |
| ATOM | 2144 | HB3 | ALA | 132 | −11.617 | 6.538 | 31.416 | 1.00 | 0.00 | 0.053 | 0.00 | 0.00 |
| ATOM | 2145 | N | SER | 133 | −13.271 | 8.773 | 30.670 | 1.00 | 0.00 | −0.650 | 9.00 | −17.40 |
| ATOM | 2146 | HN | SER | 133 | −13.214 | 8.054 | 29.934 | 1.00 | 0.00 | 0.440 | 0.00 | 0.00 |
| ATOM | 2147 | CA | SER | 133 | −13.125 | 10.156 | 30.315 | 1.00 | 0.00 | 0.158 | 9.40 | 4.00 |
| ATOM | 2148 | HA | SER | 133 | −12.303 | 10.571 | 30.899 | 1.00 | 0.00 | 0.053 | 0.00 | 0.00 |
| ATOM | 2149 | C | SER | 133 | −14.398 | 10.850 | 30.629 | 1.00 | 0.00 | 0.396 | 9.82 | 4.00 |
| ATOM | 2150 | O | SER | 133 | −14.410 | 11.999 | 31.067 | 1.00 | 0.00 | −0.396 | 8.17 | −17.40 |
| ATOM | 2151 | CB | SER | 133 | −12.833 | 10.360 | 28.819 | 1.00 | 0.00 | 0.007 | 12.77 | 4.00 |
| ATOM | 2152 | HB1 | SER | 133 | −11.942 | 9.802 | 28.530 | 1.00 | 0.00 | 0.053 | 0.00 | 0.00 |
| ATOM | 2153 | HB2 | SER | 133 | −12.668 | 11.416 | 28.609 | 1.00 | 0.00 | 0.053 | 0.00 | 0.00 |
| ATOM | 2154 | OG | SER | 133 | −13.929 | 9.905 | 28.040 | 1.00 | 0.00 | −0.537 | 11.04 | −17.40 |
| ATOM | 2155 | HG | SER | 133 | −14.505 | 10.709 | 27.754 | 1.00 | 0.00 | 0.424 | 0.00 | 0.00 |
| ATOM | 2156 | N | ARG | 134G | −15.511 | 10.144 | 30.402 | 1.00 | 0.00 | −0.650 | 9.00 | −17.40 |
| ATOM | 2157 | HN | ARG | 134G | −15.428 | 9.183 | 30.038 | 1.00 | 0.00 | 0.440 | 0.00 | 0.00 |
| ATOM | 2158 | CA | ARG | 134G | −16.807 | 10.681 | 30.648 | 1.00 | 0.00 | 0.158 | 9.40 | 4.00 |
| ATOM | 2159 | HA | ARG | 134G | −16.920 | 11.565 | 30.021 | 1.00 | 0.00 | 0.053 | 0.00 | 0.00 |
| ATOM | 2160 | C | ARG | 134G | −16.883 | 11.032 | 32.108 | 1.00 | 0.00 | 0.396 | 9.82 | 4.00 |
| ATOM | 2161 | O | ARG | 134G | −17.330 | 12.120 | 32.465 | 1.00 | 0.00 | −0.396 | 8.17 | −17.40 |
| ATOM | 2162 | CB | ARG | 134G | −17.881 | 9.635 | 30.290 | 1.00 | 0.00 | −0.106 | 12.77 | 4.00 |
| ATOM | 2163 | HB1 | ARG | 134G | −17.719 | 8.762 | 30.923 | 1.00 | 0.00 | 0.053 | 0.00 | 0.00 |
| ATOM | 2164 | HB2 | ARG | 134G | −17.761 | 9.382 | 29.236 | 1.00 | 0.00 | 0.053 | 0.00 | 0.00 |
| ATOM | 2165 | CG | ARG | 134G | −19.333 | 10.060 | 30.482 | 1.00 | 0.00 | −0.106 | 12.77 | 4.00 |
| ATOM | 2166 | HG1 | ARG | 134G | −19.955 | 9.796 | 29.626 | 1.00 | 0.00 | 0.053 | 0.00 | 0.00 |
| ATOM | 2167 | HG2 | ARG | 134G | −19.432 | 11.136 | 30.620 | 1.00 | 0.00 | 0.053 | 0.00 | 0.00 |
| ATOM | 2168 | CD | ARG | 134G | −19.971 | 9.401 | 31.707 | 1.00 | 0.00 | 0.374 | 12.77 | 4.00 |
| ATOM | 2169 | HD1 | ARG | 134G | −19.407 | 9.711 | 32.586 | 1.00 | 0.00 | 0.053 | 0.00 | 0.00 |
| ATOM | 2170 | HD2 | ARG | 134G | −19.919 | 8.320 | 31.572 | 1.00 | 0.00 | 0.053 | 0.00 | 0.00 |
| ATOM | 2171 | NE | ARG | 134G | −21.386 | 9.861 | 31.791 | 1.00 | 0.00 | −0.819 | 9.00 | −24.67 |
| ATOM | 2172 | HE | ARG | 134G | −21.852 | 10.249 | 30.959 | 1.00 | 0.00 | 0.407 | 0.00 | 0.00 |
| ATOM | 2173 | CZ | ARG | 134G | −22.062 | 9.766 | 32.974 | 1.00 | 0.00 | 0.796 | 6.95 | 4.00 |
| ATOM | 2174 | NH1 | ARG | 134G | −21.425 | 9.304 | 34.089 | 1.00 | 0.00 | −0.746 | 9.00 | −24.67 |
| ATOM | 2175 | HH1 | ARG | 134G | −21.935 | 9.232 | 34.981 | 1.00 | 0.00 | 0.407 | 0.00 | 0.00 |
| ATOM | 2176 | HH1 | ARG | 134G | −20.434 | 9.026 | 34.038 | 1.00 | 0.00 | 0.407 | 0.00 | 0.00 |
| ATOM | 2177 | NH2 | ARG | 134G | −23.377 | 10.126 | 33.045 | 1.00 | 0.00 | −0.746 | 9.00 | −24.67 |
| ATOM | 2178 | HH2 | ARG | 134G | −23.883 | 10.052 | 33.938 | 1.00 | 0.00 | 0.407 | 0.00 | 0.00 |
| ATOM | 2179 | HH2 | ARG | 134G | −23.862 | 10.470 | 32.204 | 1.00 | 0.00 | 0.407 | 0.00 | 0.00 |
| ATOM | 2180 | N | LEU | 135 | −16.436 | 10.124 | 32.998 | 1.00 | 0.00 | −0.650 | 9.00 | −17.40 |
| ATOM | 2181 | HN | LEU | 135 | −16.032 | 9.237 | 32.664 | 1.00 | 0.00 | 0.440 | 0.00 | 0.00 |
| ATOM | 2182 | CA | LEU | 135 | −16.518 | 10.384 | 34.410 | 1.00 | 0.00 | 0.158 | 9.40 | 4.00 |
| ATOM | 2183 | HA | LEU | 135 | −17.507 | 10.710 | 34.730 | 1.00 | 0.00 | 0.053 | 0.00 | 0.00 |
| ATOM | 2184 | C | LEU | 135 | −15.572 | 11.458 | 34.871 | 1.00 | 0.00 | 0.396 | 9.82 | 4.00 |
| ATOM | 2185 | O | LEU | 135 | −15.980 | 12.353 | 35.610 | 1.00 | 0.00 | −0.396 | 8.17 | −17.40 |
| ATOM | 2186 | CB | LEU | 135 | −16.285 | 9.140 | 35.289 | 1.00 | 0.00 | −0.106 | 12.77 | 4.00 |
| ATOM | 2187 | HB1 | LEU | 135 | −16.186 | 9.381 | 36.347 | 1.00 | 0.00 | 0.053 | 0.00 | 0.00 |
| ATOM | 2188 | HB2 | LEU | 135 | −15.378 | 8.596 | 35.022 | 1.00 | 0.00 | 0.053 | 0.00 | 0.00 |
| ATOM | 2189 | CG | LEU | 135 | −17.434 | 8.116 | 35.195 | 1.00 | 0.00 | −0.053 | 9.40 | 4.00 |
| ATOM | 2190 | HG | LEU | 135 | −17.502 | 7.674 | 34.200 | 1.00 | 0.00 | 0.053 | 0.00 | 0.00 |
| ATOM | 2191 | CD1 | LEU | 135 | −17.250 | 6.959 | 36.189 | 1.00 | 0.00 | −0.159 | 16.15 | 4.00 |
| ATOM | 2192 | HD1 | LEU | 135 | −18.080 | 6.259 | 36.091 | 1.00 | 0.00 | 0.053 | 0.00 | 0.00 |
| ATOM | 2193 | HD1 | LEU | 135 | −16.313 | 6.442 | 35.977 | 1.00 | 0.00 | 0.053 | 0.00 | 0.00 |
| ATOM | 2194 | HD1 | LEU | 135 | −17.225 | 7.352 | 37.205 | 1.00 | 0.00 | 0.053 | 0.00 | 0.00 |
| ATOM | 2195 | CD2 | LEU | 135 | −18.798 | 8.806 | 35.347 | 1.00 | 0.00 | −0.159 | 16.15 | 4.00 |
| ATOM | 2196 | HD2 | LEU | 135 | −19.591 | 8.062 | 35.277 | 1.00 | 0.00 | 0.053 | 0.00 | 0.00 |
| ATOM | 2197 | HD2 | LEU | 135 | −18.849 | 9.301 | 36.316 | 1.00 | 0.00 | 0.053 | 0.00 | 0.00 |
| ATOM | 2198 | HD2 | LEU | 135 | −18.922 | 9.544 | 34.555 | 1.00 | 0.00 | 0.053 | 0.00 | 0.00 |
| ATOM | 2199 | N | GLU | 136 | −14.290 | 11.428 | 34.446 | 1.00 | 0.00 | −0.650 | 9.00 | −17.40 |
| ATOM | 2200 | HN | GLU | 136 | −13.983 | 10.727 | 33.755 | 1.00 | 0.00 | 0.440 | 0.00 | 0.00 |
| ATOM | 2201 | CA | GLU | 136 | −13.359 | 12.392 | 34.974 | 1.00 | 0.00 | 0.158 | 9.40 | 4.00 |
| ATOM | 2202 | HA | GLU | 136 | −13.738 | 12.812 | 35.905 | 1.00 | 0.00 | 0.053 | 0.00 | 0.00 |
| ATOM | 2203 | C | GLU | 136 | −13.154 | 13.502 | 33.996 | 1.00 | 0.00 | 0.396 | 9.82 | 4.00 |
| ATOM | 2204 | O | GLU | 136 | −12.608 | 13.308 | 32.912 | 1.00 | 0.00 | −0.396 | 8.17 | −17.40 |
| ATOM | 2205 | CB | GLU | 136 | −11.960 | 11.820 | 35.268 | 1.00 | 0.00 | −0.106 | 12.77 | 4.00 |
| ATOM | 2206 | HB1 | GLU | 136 | −11.263 | 12.652 | 35.371 | 1.00 | 0.00 | 0.053 | 0.00 | 0.00 |
| ATOM | 2207 | HB2 | GLU | 136 | −11.669 | 11.179 | 34.435 | 1.00 | 0.00 | 0.053 | 0.00 | 0.00 |
| ATOM | 2208 | CG | GLU | 136 | −11.883 | 10.981 | 36.546 | 1.00 | 0.00 | −0.106 | 12.77 | 4.00 |
| ATOM | 2209 | HG1 | GLU | 136 | −10.960 | 10.407 | 36.511 | 1.00 | 0.00 | 0.053 | 0.00 | 0.00 |
| ATOM | 2210 | HG2 | GLU | 136 | −12.755 | 10.333 | 36.571 | 1.00 | 0.00 | 0.053 | 0.00 | 0.00 |
| ATOM | 2211 | CD | GLU | 136 | −11.883 | 11.947 | 37.728 | 1.00 | 0.00 | 0.399 | 9.82 | 4.00 |
| ATOM | 2212 | OE1 | GLU | 136 | −11.132 | 12.957 | 37.671 | 1.00 | 0.00 | −0.396 | 8.17 | −18.95 |
| ATOM | 2213 | OE2 | GLU | 136 | −12.639 | 11.686 | 38.702 | 1.00 | 0.00 | −0.427 | 8.17 | −18.95 |
| ATOM | 2215 | N | SER | 137 | −13.570 | 14.718 | 34.389 | 1.00 | 0.00 | −0.650 | 9.00 | −17.40 |
| ATOM | 2216 | HN | SER | 137 | −14.014 | 14.809 | 35.313 | 1.00 | 0.00 | 0.440 | 0.00 | 0.00 |
| ATOM | 2217 | CA | SER | 137 | −13.424 | 15.893 | 33.582 | 1.00 | 0.00 | 0.158 | 9.40 | 4.00 |
| ATOM | 2218 | HA | SER | 137 | −13.841 | 15.678 | 32.598 | 1.00 | 0.00 | 0.053 | 0.00 | 0.00 |
| ATOM | 2219 | C | SER | 137 | −11.966 | 16.208 | 33.487 | 1.00 | 0.00 | 0.396 | 9.82 | 4.00 |
| ATOM | 2220 | O | SER | 137 | −11.492 | 16.777 | 32.505 | 1.00 | 0.00 | −0.396 | 8.17 | −17.40 |
| ATOM | 2221 | CB | SER | 137 | −14.125 | 17.120 | 34.188 | 1.00 | 0.00 | 0.007 | 12.77 | 4.00 |
| ATOM | 2222 | HB1 | SER | 137 | −13.923 | 18.002 | 33.580 | 1.00 | 0.00 | 0.053 | 0.00 | 0.00 |
| ATOM | 2223 | HB2 | SER | 137 | −13.760 | 17.295 | 35.199 | 1.00 | 0.00 | 0.053 | 0.00 | 0.00 |
| ATOM | 2224 | OG | SER | 137 | −15.528 | 16.904 | 34.235 | 1.00 | 0.00 | −0.537 | 11.04 | −17.40 |
| ATOM | 2225 | HG | SER | 137 | −16.015 | 17.811 | 34.248 | 1.00 | 0.00 | 0.424 | 0.00 | 0.00 |
| ATOM | 2226 | N | VAL | 138 | −11.221 | 15.821 | 34.532 | 1.00 | 0.00 | −0.650 | 9.00 | −17.40 |
| ATOM | 2227 | HN | VAL | 138 | −11.674 | 15.254 | 35.262 | 1.00 | 0.00 | 0.440 | 0.00 | 0.00 |
| ATOM | 2228 | CA | VAL | 138 | −9.837 | 16.143 | 34.699 | 1.00 | 0.00 | 0.158 | 9.40 | 4.00 |
| ATOM | 2229 | HA | VAL | 138 | −9.656 | 17.217 | 34.712 | 1.00 | 0.00 | 0.053 | 0.00 | 0.00 |
| ATOM | 2230 | C | VAL | 138 | −8.979 | 15.587 | 33.598 | 1.00 | 0.00 | 0.396 | 9.82 | 4.00 |
| ATOM | 2231 | O | VAL | 138 | −7.983 | 16.211 | 33.239 | 1.00 | 0.00 | −0.396 | 8.17 | −17.40 |
| ATOM | 2232 | CB | VAL | 138 | −9.287 | 15.616 | 35.992 | 1.00 | 0.00 | −0.053 | 9.40 | 4.00 |
| ATOM | 2233 | HB | VAL | 138 | −9.402 | 14.532 | 36.007 | 1.00 | 0.00 | 0.053 | 0.00 | 0.00 |
| ATOM | 2234 | CG1 | VAL | 138 | −7.799 | 15.991 | 36.087 | 1.00 | 0.00 | −0.159 | 16.15 | 4.00 |
| ATOM | 2235 | HG1 | VAL | 138 | −7.387 | 15.613 | 37.023 | 1.00 | 0.00 | 0.053 | 0.00 | 0.00 |
| ATOM | 2236 | HG1 | VAL | 138 | −7.258 | 15.550 | 35.249 | 1.00 | 0.00 | 0.053 | 0.00 | 0.00 |
| ATOM | 2237 | HG1 | VAL | 138 | −7.694 | 17.075 | 36.056 | 1.00 | 0.00 | 0.053 | 0.00 | 0.00 |
| ATOM | 2238 | CG2 | VAL | 138 | −10.145 | 16.164 | 37.145 | 1.00 | 0.00 | −0.159 | 16.15 | 4.00 |
| ATOM | 2239 | HG2 | VAL | 138 | −9.759 | 15.791 | 38.094 | 1.00 | 0.00 | 0.053 | 0.00 | 0.00 |
| ATOM | 2240 | HG2 | VAL | 138 | −10.107 | 17.253 | 37.140 | 1.00 | 0.00 | 0.053 | 0.00 | 0.00 |
| ATOM | 2241 | HG2 | VAL | 138 | −11.176 | 15.836 | 37.018 | 1.00 | 0.00 | 0.053 | 0.00 | 0.00 |
| ATOM | 2242 | N | VAL | 139 | −9.309 | 14.420 | 33.009 | 1.00 | 0.00 | −0.650 | 9.00 | −17.40 |
| ATOM | 2243 | HN | VAL | 139 | −10.223 | 13.968 | 33.154 | 1.00 | 0.00 | 0.440 | 0.00 | 0.00 |
| ATOM | 2244 | CA | VAL | 139 | −8.293 | 13.849 | 32.162 | 1.00 | 0.00 | 0.158 | 9.40 | 4.00 |
| ATOM | 2245 | HA | VAL | 139 | −7.450 | 14.540 | 32.125 | 1.00 | 0.00 | 0.053 | 0.00 | 0.00 |
| ATOM | 2246 | C | VAL | 139 | −8.735 | 13.592 | 30.740 | 1.00 | 0.00 | 0.396 | 9.82 | 4.00 |
| ATOM | 2247 | O | VAL | 139 | −9.930 | 13.600 | 30.451 | 1.00 | 0.00 | −0.396 | 8.17 | −17.40 |
| ATOM | 2248 | CB | VAL | 139 | −7.840 | 12.537 | 32.723 | 1.00 | 0.00 | −0.053 | 9.40 | 4.00 |
| ATOM | 2249 | HB | VAL | 139 | −7.099 | 12.106 | 32.049 | 1.00 | 0.00 | 0.053 | 0.00 | 0.00 |
| ATOM | 2250 | CG1 | VAL | 139 | −7.221 | 12.782 | 34.109 | 1.00 | 0.00 | −0.159 | 16.15 | 4.00 |
| ATOM | 2251 | HG1 | VAL | 139 | −6.886 | 11.834 | 34.530 | 1.00 | 0.00 | 0.053 | 0.00 | 0.00 |
| ATOM | 2252 | HG1 | VAL | 139 | −6.370 | 13.457 | 34.013 | 1.00 | 0.00 | 0.053 | 0.00 | 0.00 |
| ATOM | 2253 | HG1 | VAL | 139 | −7.966 | 13.228 | 34.766 | 1.00 | 0.00 | 0.053 | 0.00 | 0.00 |
| ATOM | 2254 | CG2 | VAL | 139 | −9.038 | 11.573 | 32.740 | 1.00 | 0.00 | −0.159 | 16.15 | 4.00 |
| ATOM | 2255 | HG2 | VAL | 139 | −8.725 | 10.611 | 33.146 | 1.00 | 0.00 | 0.053 | 0.00 | 0.00 |
| ATOM | 2256 | HG2 | VAL | 139 | −9.830 | 11.988 | 33.361 | 1.00 | 0.00 | 0.053 | 0.00 | 0.00 |
| ATOM | 2257 | HG2 | VAL | 139 | −9.408 | 11.434 | 31.724 | 1.00 | 0.00 | 0.053 | 0.00 | 0.00 |
| ATOM | 2258 | N | TYR | 140 | −7.746 | 13.429 | 29.806 | 1.00 | 0.00 | −0.650 | 9.00 | −17.40 |
| ATOM | 2259 | HN | TYR | 140 | −6.776 | 13.657 | 30.068 | 1.00 | 0.00 | 0.440 | 0.00 | 0.00 |
| ATOM | 2260 | CA | TYR | 140 | −8.009 | 12.948 | 28.463 | 1.00 | 0.00 | 0.158 | 9.40 | 4.00 |
| ATOM | 2261 | HA | TYR | 140 | −8.314 | 11.904 | 28.537 | 1.00 | 0.00 | 0.053 | 0.00 | 0.00 |
| ATOM | 2262 | C | TYR | 140 | −6.837 | 13.000 | 27.482 | 1.00 | 0.00 | 0.396 | 9.82 | 4.00 |
| ATOM | 2263 | O | TYR | 140 | −6.531 | 14.076 | 26.974 | 1.00 | 0.00 | −0.396 | 8.17 | −17.40 |
| ATOM | 2264 | CB | TYR | 140 | −9.272 | 13.525 | 27.775 | 1.00 | 0.00 | −0.106 | 12.77 | 4.00 |
| ATOM | 2265 | HB1 | TYR | 140 | −10.200 | 13.247 | 28.273 | 1.00 | 0.00 | 0.053 | 0.00 | 0.00 |
| ATOM | 2266 | HB2 | TYR | 140 | −9.390 | 13.196 | 26.742 | 1.00 | 0.00 | 0.053 | 0.00 | 0.00 |
| ATOM | 2267 | CG | TYR | 140 | −9.286 | 15.016 | 27.718 | 1.00 | 0.00 | 0.000 | 7.26 | 0.60 |
| ATOM | 2268 | CD1 | TYR | 140 | −8.719 | 15.699 | 26.664 | 1.00 | 0.00 | −0.127 | 10.80 | 0.60 |
| ATOM | 2269 | HD1 | TYR | 140 | −8.243 | 15.142 | 25.856 | 1.00 | 0.00 | 0.127 | 0.00 | 0.00 |
| ATOM | 2270 | CD2 | TYR | 140 | −9.898 | 15.732 | 28.721 | 1.00 | 0.00 | −0.127 | 10.80 | 0.60 |
| ATOM | 2271 | HD2 | TYR | 140 | −10.361 | 15.206 | 29.555 | 1.00 | 0.00 | 0.127 | 0.00 | 0.00 |
| ATOM | 2272 | CE1 | TYR | 140 | −8.746 | 17.074 | 26.618 | 1.00 | 0.00 | −0.127 | 10.80 | 0.60 |
| ATOM | 2273 | HE1 | TYR | 140 | −8.287 | 17.601 | 25.781 | 1.00 | 0.00 | 0.127 | 0.00 | 0.00 |
| ATOM | 2274 | CE2 | TYR | 140 | −9.929 | 17.106 | 28.678 | 1.00 | 0.00 | −0.127 | 10.80 | 0.60 |
| ATOM | 2275 | HE2 | TYR | 140 | −10.413 | 17.662 | 29.480 | 1.00 | 0.00 | 0.127 | 0.00 | 0.00 |
| ATOM | 2276 | CZ | TYR | 140 | −9.352 | 17.781 | 27.628 | 1.00 | 0.00 | 0.026 | 7.26 | 0.60 |
| ATOM | 2277 | OH | TYR | 140 | −9.384 | 19.192 | 27.586 | 1.00 | 0.00 | −0.451 | 10.94 | −17.40 |
| ATOM | 2278 | HH | TYR | 140 | −8.421 | 19.558 | 27.604 | 1.00 | 0.00 | 0.424 | 0.00 | 0.00 |
| ATOM | 2279 | N | ALA | 141 | −6.158 | 11.842 | 27.166 | 1.00 | 0.00 | −0.650 | 9.00 | −17.40 |
| ATOM | 2280 | HN | ALA | 141 | −6.390 | 10.966 | 27.656 | 1.00 | 0.00 | 0.440 | 0.00 | 0.00 |
| ATOM | 2281 | CA | ALA | 141 | −5.111 | 11.839 | 26.143 | 1.00 | 0.00 | 0.158 | 9.40 | 4.00 |
| ATOM | 2282 | HA | ALA | 141 | −5.485 | 12.020 | 25.135 | 1.00 | 0.00 | 0.053 | 0.00 | 0.00 |
| ATOM | 2283 | C | ALA | 141 | −4.351 | 10.520 | 26.052 | 1.00 | 0.00 | 0.396 | 9.82 | 4.00 |
| ATOM | 2284 | O | ALA | 141 | −4.249 | 9.784 | 27.029 | 1.00 | 0.00 | −0.396 | 8.17 | −17.40 |
| ATOM | 2285 | CB | ALA | 141 | −4.018 | 12.954 | 26.349 | 1.00 | 0.00 | −0.159 | 16.15 | 4.00 |
| ATOM | 2286 | HB1 | ALA | 141 | −3.326 | 12.903 | 25.561 | 1.00 | 0.00 | 0.053 | 0.00 | 0.00 |
| ATOM | 2287 | HB2 | ALA | 141 | −4.576 | 13.922 | 26.313 | 1.00 | 0.00 | 0.053 | 0.00 | 0.00 |
| ATOM | 2288 | HB3 | ALA | 141 | −3.596 | 12.829 | 27.319 | 1.00 | 0.00 | 0.053 | 0.00 | 0.00 |
| ATOM | 2289 | N | SER | 142 | −3.828 | 10.183 | 24.835 | 1.00 | 0.00 | −0.650 | 9.00 | −17.40 |
| ATOM | 2290 | HN | SER | 142 | −3.993 | 10.848 | 24.066 | 1.00 | 0.00 | 0.440 | 0.00 | 0.00 |
| ATOM | 2291 | CA | SER | 142 | −3.063 | 8.990 | 24.501 | 1.00 | 0.00 | 0.158 | 9.40 | 4.00 |
| ATOM | 2292 | HA | SER | 142 | −3.496 | 8.146 | 25.038 | 1.00 | 0.00 | 0.053 | 0.00 | 0.00 |
| ATOM | 2293 | C | SER | 142 | −1.596 | 9.058 | 24.866 | 1.00 | 0.00 | 0.396 | 9.82 | 4.00 |
| ATOM | 2294 | O | SER | 142 | −1.007 | 8.040 | 25.229 | 1.00 | 0.00 | −0.396 | 8.17 | −17.40 |
| ATOM | 2295 | CB | SER | 142 | −3.106 | 8.638 | 23.006 | 1.00 | 0.00 | 0.007 | 12.77 | 4.00 |
| ATOM | 2296 | HB1 | SER | 142 | −4.115 | 8.786 | 22.622 | 1.00 | 0.00 | 0.053 | 0.00 | 0.00 |
| ATOM | 2297 | HB2 | SER | 142 | −2.817 | 7.596 | 22.865 | 1.00 | 0.00 | 0.053 | 0.00 | 0.00 |
| ATOM | 2298 | OG | SER | 142 | −2.207 | 9.473 | 22.292 | 1.00 | 0.00 | −0.537 | 11.04 | −17.40 |
| ATOM | 2299 | HG | SER | 142 | −1.418 | 9.730 | 22.902 | 1.00 | 0.00 | 0.424 | 0.00 | 0.00 |
| ATOM | 2300 | N | TRP | 143 | −0.964 | 10.248 | 24.752 | 1.00 | 0.00 | −0.650 | 9.00 | −17.40 |
| ATOM | 2301 | HN | TRP | 143 | −1.542 | 11.076 | 24.552 | 1.00 | 0.00 | 0.440 | 0.00 | 0.00 |
| ATOM | 2302 | CA | TRP | 143 | 0.465 | 10.446 | 24.889 | 1.00 | 0.00 | 0.158 | 9.40 | 4.00 |
| ATOM | 2303 | HA | TRP | 143 | 1.012 | 10.047 | 24.034 | 1.00 | 0.00 | 0.053 | 0.00 | 0.00 |
| ATOM | 2304 | C | TRP | 143 | 1.008 | 9.765 | 26.118 | 1.00 | 0.00 | 0.396 | 9.82 | 4.00 |
| ATOM | 2305 | O | TRP | 143 | 0.244 | 9.362 | 26.992 | 1.00 | 0.00 | −0.396 | 8.17 | −17.40 |
| ATOM | 2306 | CB | TRP | 143 | 0.837 | 11.937 | 24.969 | 1.00 | 0.00 | −0.106 | 12.77 | 4.00 |
| ATOM | 2307 | HB1 | TRP | 143 | 0.400 | 12.349 | 25.878 | 1.00 | 0.00 | 0.053 | 0.00 | 0.00 |
| ATOM | 2308 | HB2 | TRP | 143 | 0.434 | 12.437 | 24.088 | 1.00 | 0.00 | 0.053 | 0.00 | 0.00 |
| ATOM | 2309 | CG | TRP | 143 | 2.319 | 12.208 | 25.010 | 1.00 | 0.00 | 0.000 | 7.26 | 0.60 |
| ATOM | 2310 | CD1 | TRP | 143 | 3.162 | 12.302 | 26.077 | 1.00 | 0.00 | −0.177 | 10.80 | 0.60 |
| ATOM | 2311 | HD1 | TRP | 143 | 2.867 | 12.182 | 27.119 | 1.00 | 0.00 | 0.127 | 0.00 | 0.00 |
| ATOM | 2312 | CD2 | TRP | 143 | 3.115 | 12.435 | 23.839 | 1.00 | 0.00 | 0.000 | 6.80 | 0.60 |
| ATOM | 2313 | NE1 | TRP | 143 | 4.439 | 12.570 | 25.642 | 1.00 | 0.00 | −0.292 | 9.00 | −17.40 |
| ATOM | 2314 | HE1 | TRP | 143 | 5.267 | 12.687 | 26.243 | 1.00 | 0.00 | 0.393 | 0.00 | 0.00 |
| ATOM | 2315 | CE2 | TRP | 143 | 4.424 | 12.655 | 24.265 | 1.00 | 0.00 | −0.050 | 6.80 | 0.60 |
| ATOM | 2316 | CE3 | TRP | 143 | 2.783 | 12.453 | 22.514 | 1.00 | 0.00 | −0.127 | 10.80 | 0.60 |
| ATOM | 2317 | HD3 | TRP | 143 | 1.759 | 12.277 | 22.182 | 1.00 | 0.00 | 0.127 | 0.00 | 0.00 |
| ATOM | 2318 | CZ2 | TRP | 143 | 5.427 | 12.896 | 23.370 | 1.00 | 0.00 | −0.127 | 10.80 | 0.60 |
| ATOM | 2319 | HZ2 | TRP | 143 | 6.452 | 13.062 | 23.700 | 1.00 | 0.00 | 0.127 | 0.00 | 0.00 |
| ATOM | 2320 | CZ3 | TRP | 143 | 3.796 | 12.703 | 21.615 | 1.00 | 0.00 | −0.127 | 10.80 | 0.60 |
| ATOM | 2321 | HZ3 | TRP | 143 | 3.568 | 12.730 | 20.549 | 1.00 | 0.00 | 0.127 | 0.00 | 0.00 |
| ATOM | 2322 | CH2 | TRP | 143 | 5.093 | 12.920 | 22.034 | 1.00 | 0.00 | −0.127 | 10.80 | 0.60 |
| ATOM | 2323 | HH2 | TRP | 143 | 5.868 | 13.114 | 21.292 | 1.00 | 0.00 | 0.127 | 0.00 | 0.00 |
| ATOM | 2324 | N | SER | 144 | 2.358 | 9.599 | 26.211 | 1.00 | 0.00 | −0.650 | 9.00 | −17.40 |
| ATOM | 2325 | HN | SER | 144 | 2.971 | 9.941 | 25.457 | 1.00 | 0.00 | 0.440 | 0.00 | 0.00 |
| ATOM | 2326 | CA | SER | 144 | 2.929 | 8.944 | 27.365 | 1.00 | 0.00 | 0.158 | 9.40 | 4.00 |
| ATOM | 2327 | HA | SER | 144 | 2.423 | 9.228 | 28.287 | 1.00 | 0.00 | 0.053 | 0.00 | 0.00 |
| ATOM | 2328 | C | SER | 144 | 4.389 | 9.285 | 27.544 | 1.00 | 0.00 | 0.396 | 9.82 | 4.00 |
| ATOM | 2329 | O | SER | 144 | 5.014 | 9.908 | 26.686 | 1.00 | 0.00 | −0.396 | 8.17 | −17.40 |
| ATOM | 2330 | CB | SER | 144 | 2.889 | 7.413 | 27.268 | 1.00 | 0.00 | 0.007 | 12.77 | 4.00 |
| ATOM | 2331 | HB1 | SER | 144 | 1.888 | 7.085 | 26.986 | 1.00 | 0.00 | 0.053 | 0.00 | 0.00 |
| ATOM | 2332 | HB2 | SER | 144 | 3.149 | 6.973 | 28.230 | 1.00 | 0.00 | 0.053 | 0.00 | 0.00 |
| ATOM | 2333 | OG | SER | 144 | 3.815 | 6.967 | 26.289 | 1.00 | 0.00 | −0.537 | 11.04 | −17.40 |
| ATOM | 2334 | HG | SER | 144 | 3.734 | 7.554 | 25.446 | 1.00 | 0.00 | 0.424 | 0.00 | 0.00 |
| ATOM | 2335 | N | ARG | 145G | 4.945 | 8.885 | 28.719 | 1.00 | 0.00 | −0.650 | 9.00 | −17.40 |
| ATOM | 2336 | HN | ARG | 145G | 4.317 | 8.450 | 29.410 | 1.00 | 0.00 | 0.440 | 0.00 | 0.00 |
| ATOM | 2337 | CA | ARG | 145G | 6.335 | 9.015 | 29.084 | 1.00 | 0.00 | 0.158 | 9.40 | 4.00 |
| ATOM | 2338 | HA | ARG | 145G | 6.903 | 8.503 | 28.307 | 1.00 | 0.00 | 0.053 | 0.00 | 0.00 |
| ATOM | 2339 | C | ARG | 145G | 6.480 | 8.362 | 30.423 | 1.00 | 0.00 | 0.396 | 9.82 | 4.00 |
| ATOM | 2340 | O | ARG | 145G | 6.614 | 9.054 | 31.432 | 1.00 | 0.00 | −0.396 | 8.17 | −17.40 |
| ATOM | 2341 | CB | ARG | 145G | 6.788 | 10.479 | 29.219 | 1.00 | 0.00 | −0.106 | 12.77 | 4.00 |
| ATOM | 2342 | HB1 | ARG | 145G | 6.136 | 10.972 | 29.939 | 1.00 | 0.00 | 0.053 | 0.00 | 0.00 |
| ATOM | 2343 | HB2 | ARG | 145G | 6.708 | 10.952 | 28.240 | 1.00 | 0.00 | 0.053 | 0.00 | 0.00 |
| ATOM | 2344 | CG | ARG | 145G | 8.229 | 10.656 | 29.702 | 1.00 | 0.00 | −0.106 | 12.77 | 4.00 |
| ATOM | 2345 | HG1 | ARG | 145G | 8.957 | 10.159 | 29.060 | 1.00 | 0.00 | 0.053 | 0.00 | 0.00 |
| ATOM | 2346 | HG2 | ARG | 145G | 8.395 | 10.255 | 30.702 | 1.00 | 0.00 | 0.053 | 0.00 | 0.00 |
| ATOM | 2347 | CD | ARG | 145G | 8.663 | 12.123 | 29.765 | 1.00 | 0.00 | 0.374 | 12.77 | 4.00 |
| ATOM | 2348 | HD1 | ARG | 145G | 7.940 | 12.661 | 30.378 | 1.00 | 0.00 | 0.053 | 0.00 | 0.00 |
| ATOM | 2349 | HD2 | ARG | 145G | 8.677 | 12.513 | 28.747 | 1.00 | 0.00 | 0.053 | 0.00 | 0.00 |
| ATOM | 2350 | NE | ARG | 145G | 10.023 | 12.175 | 30.374 | 1.00 | 0.00 | −0.819 | 9.00 | −24.67 |
| ATOM | 2351 | HE | ARG | 145G | 10.857 | 12.032 | 29.787 | 1.00 | 0.00 | 0.407 | 0.00 | 0.00 |
| ATOM | 2352 | CZ | ARG | 145G | 10.158 | 12.412 | 31.713 | 1.00 | 0.00 | 0.796 | 6.95 | 4.00 |
| ATOM | 2353 | NH1 | ARG | 145G | 9.053 | 12.598 | 32.492 | 1.00 | 0.00 | −0.746 | 9.00 | −24.67 |
| ATOM | 2354 | HH1 | ARG | 145G | 9.158 | 12.776 | 33.500 | 1.00 | 0.00 | 0.407 | 0.00 | 0.00 |
| ATOM | 2355 | HH1 | ARG | 145G | 8.113 | 12.560 | 32.071 | 1.00 | 0.00 | 0.407 | 0.00 | 0.00 |
| ATOM | 2356 | NH2 | ARG | 145G | 11.399 | 12.470 | 32.280 | 1.00 | 0.00 | −0.746 | 9.00 | −24.67 |
| ATOM | 2357 | HH2 | ARG | 145G | 11.496 | 12.648 | 33.289 | 1.00 | 0.00 | 0.407 | 0.00 | 0.00 |
| ATOM | 2358 | HH2 | ARG | 145G | 12.237 | 12.334 | 31.698 | 1.00 | 0.00 | 0.407 | 0.00 | 0.00 |
| ATOM | 2359 | N | VAL | 146 | 6.474 | 7.008 | 30.452 | 1.00 | 0.00 | −0.650 | 9.00 | −17.40 |
| ATOM | 2360 | HN | VAL | 146 | 6.432 | 6.493 | 29.560 | 1.00 | 0.00 | 0.440 | 0.00 | 0.00 |
| ATOM | 2361 | CA | VAL | 146 | 6.524 | 6.260 | 31.681 | 1.00 | 0.00 | 0.158 | 9.40 | 4.00 |
| ATOM | 2362 | HA | VAL | 146 | 7.291 | 6.662 | 32.342 | 1.00 | 0.00 | 0.053 | 0.00 | 0.00 |
| ATOM | 2363 | C | VAL | 146 | 6.842 | 4.818 | 31.407 | 1.00 | 0.00 | 0.396 | 9.82 | 4.00 |
| ATOM | 2364 | O | VAL | 146 | 7.651 | 4.493 | 30.539 | 1.00 | 0.00 | −0.396 | 8.17 | −17.40 |
| ATOM | 2365 | CB | VAL | 146 | 5.234 | 6.263 | 32.450 | 1.00 | 0.00 | −0.053 | 9.40 | 4.00 |
| ATOM | 2366 | HB | VAL | 146 | 5.251 | 5.501 | 33.230 | 1.00 | 0.00 | 0.053 | 0.00 | 0.00 |
| ATOM | 2367 | CG1 | VAL | 146 | 5.004 | 7.630 | 33.113 | 1.00 | 0.00 | −0.159 | 16.15 | 4.00 |
| ATOM | 2368 | HG1 | VAL | 146 | 4.064 | 7.612 | 33.665 | 1.00 | 0.00 | 0.053 | 0.00 | 0.00 |
| ATOM | 2369 | HG1 | VAL | 146 | 5.824 | 7.844 | 33.798 | 1.00 | 0.00 | 0.053 | 0.00 | 0.00 |
| ATOM | 2370 | HG1 | VAL | 146 | 4.959 | 8.403 | 32.346 | 1.00 | 0.00 | 0.053 | 0.00 | 0.00 |
| ATOM | 2371 | CG2 | VAL | 146 | 4.127 | 5.903 | 31.454 | 1.00 | 0.00 | −0.159 | 16.15 | 4.00 |
| ATOM | 2372 | HG2 | VAL | 146 | 3.165 | 5.892 | 31.967 | 1.00 | 0.00 | 0.053 | 0.00 | 0.00 |
| ATOM | 2373 | HG2 | VAL | 146 | 4.102 | 6.642 | 30.653 | 1.00 | 0.00 | 0.053 | 0.00 | 0.00 |
| ATOM | 2374 | HG2 | VAL | 146 | 4.324 | 4.917 | 31.031 | 1.00 | 0.00 | 0.053 | 0.00 | 0.00 |
| ATOM | 2375 | N | GLN | 147 | 6.201 | 3.920 | 32.195 | 1.00 | 0.00 | −0.650 | 9.00 | −17.40 |
| ATOM | 2376 | HN | GLN | 147 | 5.487 | 4.276 | 32.846 | 1.00 | 0.00 | 0.440 | 0.00 | 0.00 |
| ATOM | 2377 | CA | GLN | 147 | 6.449 | 2.501 | 32.183 | 1.00 | 0.00 | 0.158 | 9.40 | 4.00 |
| ATOM | 2378 | HA | GLN | 147 | 7.517 | 2.300 | 32.098 | 1.00 | 0.00 | 0.053 | 0.00 | 0.00 |
| ATOM | 2379 | C | GLN | 147 | 5.749 | 1.852 | 31.016 | 1.00 | 0.00 | 0.396 | 9.82 | 4.00 |
| ATOM | 2380 | O | GLN | 147 | 5.372 | 2.512 | 30.049 | 1.00 | 0.00 | −0.396 | 8.17 | −17.40 |
| ATOM | 2381 | CB | GLN | 147 | 6.011 | 1.808 | 33.494 | 1.00 | 0.00 | −0.106 | 12.77 | 4.00 |
| ATOM | 2382 | HB1 | GLN | 147 | 4.926 | 1.704 | 33.478 | 1.00 | 0.00 | 0.053 | 0.00 | 0.00 |
| ATOM | 2383 | HB2 | GLN | 147 | 6.327 | 2.431 | 34.330 | 1.00 | 0.00 | 0.053 | 0.00 | 0.00 |
| ATOM | 2384 | CG | GLN | 147 | 6.604 | 0.411 | 33.710 | 1.00 | 0.00 | −0.106 | 12.77 | 4.00 |
| ATOM | 2385 | HG1 | GLN | 147 | 7.689 | 0.502 | 33.730 | 1.00 | 0.00 | 0.053 | 0.00 | 0.00 |
| ATOM | 2386 | HG2 | GLN | 147 | 6.285 | −0.224 | 32.883 | 1.00 | 0.00 | 0.053 | 0.00 | 0.00 |
| ATOM | 2387 | CD | GLN | 147 | 6.082 | −0.126 | 35.037 | 1.00 | 0.00 | 0.396 | 9.82 | 4.00 |
| ATOM | 2388 | OE1 | GLN | 147 | 6.126 | −0.537 | 33.879 | 1.00 | 0.00 | −0.396 | 8.17 | −17.40 |
| ATOM | 2389 | NE2 | GLN | 147 | 6.465 | 0.979 | 35.732 | 1.00 | 0.00 | −0.879 | 13.25 | −17.40 |
| ATOM | 2390 | HE2 | GLN | 147 | 6.283 | 1.039 | 36.744 | 1.00 | 0.00 | 0.440 | 0.00 | 0.00 |
| ATOM | 2391 | HE2 | GLN | 147 | 6.936 | 1.755 | 35.246 | 1.00 | 0.00 | 0.440 | 0.00 | 0.00 |
| ATOM | 2392 | N | ALA | 148 | 5.618 | 0.506 | 31.076 | 1.00 | 0.00 | −0.650 | 9.00 | −17.40 |
| ATOM | 2393 | HN | ALA | 148 | 5.902 | 0.038 | 31.948 | 1.00 | 0.00 | 0.440 | 0.00 | 0.00 |
| ATOM | 2394 | CA | ALA | 148 | 5.112 | −0.320 | 30.013 | 1.00 | 0.00 | 0.158 | 9.40 | 4.00 |
| ATOM | 2395 | HA | ALA | 148 | 5.682 | −0.091 | 29.112 | 1.00 | 0.00 | 0.053 | 0.00 | 0.00 |
| ATOM | 2396 | C | ALA | 148 | 3.656 | −0.142 | 29.669 | 1.00 | 0.00 | 0.396 | 9.82 | 4.00 |
| ATOM | 2397 | O | ALA | 148 | 3.354 | 0.230 | 28.537 | 1.00 | 0.00 | −0.396 | 8.17 | −17.40 |
| ATOM | 2398 | CB | ALA | 148 | 5.312 | −1.819 | 30.296 | 1.00 | 0.00 | −0.159 | 16.15 | 4.00 |
| ATOM | 2399 | HB1 | ALA | 148 | 4.914 | −2.402 | 29.465 | 1.00 | 0.00 | 0.053 | 0.00 | 0.00 |
| ATOM | 2400 | HB2 | ALA | 148 | 6.375 | −2.029 | 30.410 | 1.00 | 0.00 | 0.053 | 0.00 | 0.00 |
| ATOM | 2401 | HB3 | ALA | 148 | 4.788 | −2.089 | 31.212 | 1.00 | 0.00 | 0.053 | 0.00 | 0.00 |
| ATOM | 2402 | N | ASP | 149P | 2.706 | −0.356 | 30.611 | 1.00 | 0.00 | −0.650 | 9.00 | −17.40 |
| ATOM | 2403 | HN | ASP | 149P | 2.959 | −0.509 | 31.597 | 1.00 | 0.00 | 0.440 | 0.00 | 0.00 |
| ATOM | 2404 | CA | ASP | 149P | 1.323 | −0.362 | 30.183 | 1.00 | 0.00 | 0.158 | 9.40 | 4.00 |
| ATOM | 2405 | HA | ASP | 149P | 1.216 | 0.208 | 29.260 | 1.00 | 0.00 | 0.053 | 0.00 | 0.00 |
| ATOM | 2406 | C | ASP | 149P | 0.459 | 0.254 | 31.238 | 1.00 | 0.00 | 0.396 | 9.82 | 4.00 |
| ATOM | 2407 | O | ASP | 149P | −0.762 | 0.109 | 31.196 | 1.00 | 0.00 | −0.396 | 8.17 | −17.40 |
| ATOM | 2408 | CB | ASP | 149P | 0.757 | −1.790 | 30.047 | 1.00 | 0.00 | −0.336 | 12.77 | 4.00 |
| ATOM | 2409 | HB1 | ASP | 149P | −0.306 | −1.776 | 29.808 | 1.00 | 0.00 | 0.053 | 0.00 | 0.00 |
| ATOM | 2410 | HB2 | ASP | 149P | 0.876 | −2.356 | 30.970 | 1.00 | 0.00 | 0.053 | 0.00 | 0.00 |
| ATOM | 2411 | CG | ASP | 149P | 1.462 | −2.567 | 28.941 | 1.00 | 0.00 | 0.297 | 9.82 | 4.00 |
| ATOM | 2412 | OD1 | ASP | 149P | 2.122 | −1.934 | 28.076 | 1.00 | 0.00 | −0.534 | 8.17 | −18.95 |
| ATOM | 2413 | OD2 | ASP | 149P | 1.332 | −3.820 | 28.943 | 1.00 | 0.00 | −0.534 | 8.17 | −18.95 |
| ATOM | 2414 | N | LEU | 150 | 1.044 | 0.978 | 32.202 | 1.00 | 0.00 | −0.650 | 9.00 | −17.40 |
| ATOM | 2415 | HN | LEU | 150 | 2.058 | 1.155 | 32.193 | 1.00 | 0.00 | 0.440 | 0.00 | 0.00 |
| ATOM | 2416 | CA | LEU | 150 | 0.211 | 1.496 | 33.245 | 1.00 | 0.00 | 0.158 | 9.40 | 4.00 |
| ATOM | 2417 | HA | LEU | 150 | −0.429 | 0.697 | 33.619 | 1.00 | 0.00 | 0.053 | 0.00 | 0.00 |
| ATOM | 2418 | C | LEU | 150 | −0.625 | 2.601 | 32.697 | 1.00 | 0.00 | 0.396 | 9.82 | 4.00 |
| ATOM | 2419 | O | LEU | 150 | −0.738 | 2.791 | 31.487 | 1.00 | 0.00 | −0.396 | 8.17 | −17.40 |
| ATOM | 2420 | CB | LEU | 150 | 0.992 | 2.056 | 34.447 | 1.00 | 0.00 | −0.106 | 12.77 | 4.00 |
| ATOM | 2421 | HB1 | LEU | 150 | 0.359 | 2.549 | 35.184 | 1.00 | 0.00 | 0.053 | 0.00 | 0.00 |
| ATOM | 2422 | HB2 | LEU | 150 | 1.740 | 2.798 | 34.171 | 1.00 | 0.00 | 0.053 | 0.00 | 0.00 |
| ATOM | 2423 | CG | LEU | 150 | 1.762 | 0.982 | 35.233 | 1.00 | 0.00 | −0.053 | 9.40 | 4.00 |
| ATOM | 2424 | HG | LEU | 150 | 1.097 | 0.187 | 35.571 | 1.00 | 0.00 | 0.053 | 0.00 | 0.00 |
| ATOM | 2425 | CD1 | LEU | 150 | 2.856 | 0.337 | 34.370 | 1.00 | 0.00 | −0.159 | 16.15 | 4.00 |
| ATOM | 2426 | HD1 | LEU | 150 | 3.382 | −0.417 | 34.954 | 1.00 | 0.00 | 0.053 | 0.00 | 0.00 |
| ATOM | 2427 | HD1 | LEU | 150 | 2.401 | −0.131 | 33.496 | 1.00 | 0.00 | 0.053 | 0.00 | 0.00 |
| ATOM | 2428 | HD1 | LEU | 150 | 3.561 | 1.101 | 34.045 | 1.00 | 0.00 | 0.053 | 0.00 | 0.00 |
| ATOM | 2429 | CD2 | LEU | 150 | 2.300 | 1.541 | 36.560 | 1.00 | 0.00 | −0.159 | 16.15 | 4.00 |
| ATOM | 2430 | HD2 | LEU | 150 | 2.839 | 0.757 | 37.092 | 1.00 | 0.00 | 0.053 | 0.00 | 0.00 |
| ATOM | 2431 | HD2 | LEU | 150 | 2.974 | 2.373 | 36.357 | 1.00 | 0.00 | 0.053 | 0.00 | 0.00 |
| ATOM | 2432 | HD2 | LEU | 150 | 1.468 | 1.889 | 37.172 | 1.00 | 0.00 | 0.053 | 0.00 | 0.00 |
| ATOM | 2433 | N | ASN | 151 | −1.305 | 3.316 | 33.608 | 1.00 | 0.00 | −0.650 | 9.00 | −17.40 |
| ATOM | 2434 | HN | ASN | 151 | −1.259 | 3.037 | 34.598 | 1.00 | 0.00 | 0.440 | 0.00 | 0.00 |
| ATOM | 2435 | CA | ASN | 151 | −2.090 | 4.452 | 33.242 | 1.00 | 0.00 | 0.158 | 9.40 | 4.00 |
| ATOM | 2436 | HA | ASN | 151 | −1.833 | 4.796 | 32.240 | 1.00 | 0.00 | 0.053 | 0.00 | 0.00 |
| ATOM | 2437 | C | ASN | 151 | −1.746 | 5.471 | 34.281 | 1.00 | 0.00 | 0.396 | 9.82 | 4.00 |
| ATOM | 2438 | O | ASN | 151 | −1.506 | 5.115 | 35.433 | 1.00 | 0.00 | −0.396 | 8.17 | −17.40 |
| ATOM | 2439 | CB | ASN | 151 | −3.598 | 4.169 | 33.321 | 1.00 | 0.00 | −0.106 | 12.77 | 4.00 |
| ATOM | 2440 | HB1 | ASN | 151 | −4.118 | 5.087 | 33.049 | 1.00 | 0.00 | 0.053 | 0.00 | 0.00 |
| ATOM | 2441 | HB2 | ASN | 151 | −3.823 | 3.873 | 34.345 | 1.00 | 0.00 | 0.053 | 0.00 | 0.00 |
| ATOM | 2442 | CG | ASN | 151 | −3.906 | 3.045 | 32.338 | 1.00 | 0.00 | 0.396 | 9.82 | 4.00 |
| ATOM | 2443 | OD1 | ASN | 151 | −4.417 | 3.262 | 31.241 | 1.00 | 0.00 | −0.396 | 8.17 | −17.40 |
| ATOM | 2444 | ND2 | ASN | 151 | −3.567 | 1.791 | 32.740 | 1.00 | 0.00 | −0.879 | 13.25 | −17.40 |
| ATOM | 2445 | HD2 | ASN | 151 | −3.140 | 1.644 | 33.666 | 1.00 | 0.00 | 0.440 | 0.00 | 0.00 |
| ATOM | 2446 | HD2 | ASN | 151 | −3.736 | 0.987 | 32.118 | 1.00 | 0.00 | 0.440 | 0.00 | 0.00 |
| ATOM | 2447 | N | CYS | 152S | −1.673 | 6.767 | 33.918 | 1.00 | 0.00 | −0.65 | 9.00 | −17.40 |
| ATOM | 2448 | HN | CYS | 152S | −1.879 | 7.071 | 32.956 | 1.00 | 0.00 | 0.44 | 0.00 | 0.00 |
| ATOM | 2449 | CA | CYS | 152S | −1.294 | 7.700 | 34.939 | 1.00 | 0.00 | 0.15 | 9.40 | 4.00 |
| ATOM | 2450 | HA | CYS | 152S | −1.745 | 7.385 | 35.879 | 1.00 | 0.00 | 0.05 | 0.00 | 0.00 |
| ATOM | 2451 | C | CYS | 152S | −1.790 | 9.058 | 34.549 | 1.00 | 0.00 | 0.39 | 9.82 | 4.00 |
| ATOM | 2452 | O | CYS | 152S | −2.460 | 9.220 | 33.531 | 1.00 | 0.00 | −0.39 | 8.17 | −17.40 |
| ATOM | 2453 | CB | CYS | 152S | 0.233 | 7.782 | 35.130 | 1.00 | 0.00 | −0.04 | 12.77 | 4.00 |
| ATOM | 2454 | HB1 | CYS | 152S | 0.740 | 8.328 | 34.335 | 1.00 | 0.00 | 0.05 | 0.00 | 0.00 |
| ATOM | 2455 | HB2 | CYS | 152S | 0.719 | 6.807 | 35.162 | 1.00 | 0.00 | 0.05 | 0.00 | 0.00 |
| ATOM | 2456 | SG | CYS | 152S | 0.726 | 8.612 | 36.670 | 1.00 | 0.00 | −0.25 | 19.93 | 6.40 |
| ATOM | 2457 | HG | CYS | 152S | 1.424 | 9.718 | 36.378 | 1.00 | 0.00 | 0.19 | 0.00 | 0.00 |
| ATOM | 2458 | N | MET | 153 | −1.490 | 10.070 | 35.389 | 1.00 | 0.00 | −0.650 | 9.00 | −17.40 |
| ATOM | 2459 | HN | MET | 153 | −0.966 | 9.864 | 36.251 | 1.00 | 0.00 | 0.440 | 0.00 | 0.00 |
| ATOM | 2460 | CA | MET | 153 | −1.878 | 11.426 | 35.117 | 1.00 | 0.00 | 0.158 | 9.40 | 4.00 |
| ATOM | 2461 | HA | MET | 153 | −2.658 | 11.365 | 34.358 | 1.00 | 0.00 | 0.053 | 0.00 | 0.00 |
| ATOM | 2462 | C | MET | 153 | −0.638 | 12.116 | 34.625 | 1.00 | 0.00 | 0.396 | 9.82 | 4.00 |
| ATOM | 2463 | O | MET | 153 | 0.436 | 11.918 | 35.189 | 1.00 | 0.00 | −0.396 | 8.17 | −17.40 |
| ATOM | 2464 | CB | MET | 153 | −2.308 | 12.205 | 36.374 | 1.00 | 0.00 | −0.106 | 12.77 | 4.00 |
| ATOM | 2465 | HB1 | MET | 153 | −2.522 | 13.232 | 36.078 | 1.00 | 0.00 | 0.053 | 0.00 | 0.00 |
| ATOM | 2466 | HB2 | MET | 153 | −1.487 | 12.171 | 37.090 | 1.00 | 0.00 | 0.053 | 0.00 | 0.00 |
| ATOM | 2467 | CG | MET | 153 | −3.553 | 11.663 | 37.080 | 1.00 | 0.00 | −0.041 | 12.77 | 4.00 |
| ATOM | 2468 | HG1 | MET | 153 | −3.660 | 12.189 | 38.028 | 1.00 | 0.00 | 0.053 | 0.00 | 0.00 |
| ATOM | 2469 | HG2 | MET | 153 | −3.411 | 10.594 | 37.243 | 1.00 | 0.00 | 0.053 | 0.00 | 0.00 |
| ATOM | 2470 | SD | MET | 153 | −5.107 | 11.871 | 36.164 | 1.00 | 0.00 | −0.130 | 16.39 | 6.40 |
| ATOM | 2471 | CE | MET | 153 | −4.721 | 10.572 | 34.957 | 1.00 | 0.00 | −0.094 | 16.15 | 4.00 |
| ATOM | 2472 | HE1 | MET | 153 | −5.540 | 10.482 | 34.244 | 1.00 | 0.00 | 0.053 | 0.00 | 0.00 |
| ATOM | 2473 | HE2 | MET | 153 | −3.804 | 10.829 | 34.425 | 1.00 | 0.00 | 0.053 | 0.00 | 0.00 |
| ATOM | 2474 | HE3 | MET | 153 | −4.585 | 9.622 | 35.475 | 1.00 | 0.00 | 0.053 | 0.00 | 0.00 |
| ATOM | 2475 | N | LYS | 154S | −0.740 | 12.932 | 33.551 | 1.00 | 0.00 | −0.650 | 9.00 | −17.40 |
| ATOM | 2476 | HN | LYS | 154S | −1.656 | 13.073 | 33.103 | 1.00 | 0.00 | 0.440 | 0.00 | 0.00 |
| ATOM | 2477 | CA | LYS | 154S | 0.421 | 13.606 | 33.027 | 1.00 | 0.00 | 0.158 | 9.40 | 4.00 |
| ATOM | 2478 | HA | LYS | 154S | 1.225 | 13.538 | 33.759 | 1.00 | 0.00 | 0.053 | 0.00 | 0.00 |
| ATOM | 2479 | C | LYS | 154S | 0.070 | 15.043 | 32.773 | 1.00 | 0.00 | 0.396 | 9.82 | 4.00 |
| ATOM | 2480 | O | LYS | 154S | −0.904 | 15.344 | 32.086 | 1.00 | 0.00 | −0.396 | 8.17 | −17.40 |
| ATOM | 2481 | CB | LYS | 154S | 0.903 | 13.006 | 31.693 | 1.00 | 0.00 | −0.106 | 12.77 | 4.00 |
| ATOM | 2482 | HB1 | LYS | 154S | 0.115 | 13.146 | 30.953 | 1.00 | 0.00 | 0.053 | 0.00 | 0.00 |
| ATOM | 2483 | HB2 | LYS | 154S | 1.101 | 11.945 | 31.849 | 1.00 | 0.00 | 0.053 | 0.00 | 0.00 |
| ATOM | 2484 | CG | LYS | 154S | 2.181 | 13.634 | 31.128 | 1.00 | 0.00 | −0.106 | 12.77 | 4.00 |
| ATOM | 2485 | HG1 | LYS | 154S | 2.959 | 13.756 | 31.881 | 1.00 | 0.00 | 0.053 | 0.00 | 0.00 |
| ATOM | 2486 | HG2 | LYS | 154S | 2.013 | 14.624 | 30.706 | 1.00 | 0.00 | 0.053 | 0.00 | 0.00 |
| ATOM | 2487 | CD | LYS | 154S | 2.807 | 12.798 | 30.006 | 1.00 | 0.00 | −0.106 | 12.77 | 4.00 |
| ATOM | 2488 | HD1 | LYS | 154S | 2.076 | 12.698 | 29.202 | 1.00 | 0.00 | 0.053 | 0.00 | 0.00 |
| ATOM | 2489 | HD2 | LYS | 154S | 3.067 | 11.819 | 30.409 | 1.00 | 0.00 | 0.053 | 0.00 | 0.00 |
| ATOM | 2490 | CE | LYS | 154S | 4.077 | 13.412 | 29.411 | 1.00 | 0.00 | 0.099 | 12.77 | 4.00 |
| ATOM | 2491 | HE1 | LYS | 154S | 4.767 | 13.687 | 30.208 | 1.00 | 0.00 | 0.053 | 0.00 | 0.00 |
| ATOM | 2492 | HE2 | LYS | 154S | 3.826 | 14.303 | 28.836 | 1.00 | 0.00 | 0.053 | 0.00 | 0.00 |
| ATOM | 2493 | NZ | LYS | 154S | 4.742 | 12.436 | 28.517 | 1.00 | 0.00 | −0.045 | 13.25 | 39.20 |
| ATOM | 2494 | HZ1 | LYS | 154S | 5.594 | 12.857 | 28.121 | 1.00 | 0.00 | 0.280 | 0.00 | 0.00 |
| ATOM | 2495 | HZ2 | LYS | 154S | 4.993 | 11.593 | 29.053 | 1.00 | 0.00 | 0.280 | 0.00 | 0.00 |
| ATOM | 2496 | HZ3 | LYS | 154S | 4.101 | 12.177 | 27.753 | 1.00 | 0.00 | 0.280 | 0.00 | 0.00 |
| ATOM | 2497 | N | ASP | 155P | 0.863 | 15.970 | 33.345 | 1.00 | 0.00 | −0.650 | 9.00 | −17.40 |
| ATOM | 2498 | HN | ASP | 155P | 1.674 | 15.650 | 33.892 | 1.00 | 0.00 | 0.440 | 0.00 | 0.00 |
| ATOM | 2499 | CA | ASP | 155P | 0.631 | 17.384 | 33.228 | 1.00 | 0.00 | 0.158 | 9.40 | 4.00 |
| ATOM | 2500 | HA | ASP | 155P | −0.404 | 17.657 | 33.433 | 1.00 | 0.00 | 0.053 | 0.00 | 0.00 |
| ATOM | 2501 | C | ASP | 155P | 0.937 | 17.908 | 31.854 | 1.00 | 0.00 | 0.396 | 9.82 | 4.00 |
| ATOM | 2502 | O | ASP | 155P | 0.171 | 18.697 | 31.303 | 1.00 | 0.00 | −0.396 | 8.17 | −17.40 |
| ATOM | 2503 | CB | ASP | 155P | 1.480 | 18.197 | 34.218 | 1.00 | 0.00 | −0.336 | 12.77 | 4.00 |
| ATOM | 2504 | HB1 | ASP | 155P | 1.373 | 19.249 | 33.955 | 1.00 | 0.00 | 0.053 | 0.00 | 0.00 |
| ATOM | 2505 | HB2 | ASP | 155P | 2.513 | 17.865 | 34.114 | 1.00 | 0.00 | 0.053 | 0.00 | 0.00 |
| ATOM | 2506 | CG | ASP | 155P | 0.947 | 17.919 | 35.615 | 1.00 | 0.00 | 0.297 | 9.82 | 4.00 |
| ATOM | 2507 | OD1 | ASP | 155P | −0.269 | 17.610 | 35.732 | 1.00 | 0.00 | −0.534 | 8.17 | −18.95 |
| ATOM | 2508 | OD2 | ASP | 155P | 1.748 | 18.006 | 36.582 | 1.00 | 0.00 | −0.534 | 8.17 | −18.95 |
| ATOM | 2509 | N | LEU | 156 | 2.061 | 17.479 | 31.247 | 1.00 | 0.00 | −0.650 | 9.00 | −17.40 |
| ATOM | 2510 | HN | LEU | 156 | 2.621 | 16.722 | 31.664 | 1.00 | 0.00 | 0.440 | 0.00 | 0.00 |
| ATOM | 2511 | CA | LEU | 156 | 2.470 | 18.088 | 30.011 | 1.00 | 0.00 | 0.158 | 9.40 | 4.00 |
| ATOM | 2512 | HA | LEU | 156 | 2.484 | 19.170 | 30.134 | 1.00 | 0.00 | 0.053 | 0.00 | 0.00 |
| ATOM | 2513 | C | LEU | 156 | 1.502 | 17.714 | 28.939 | 1.00 | 0.00 | 0.396 | 9.82 | 4.00 |
| ATOM | 2514 | O | LEU | 156 | 1.105 | 16.557 | 28.816 | 1.00 | 0.00 | −0.396 | 8.17 | −17.40 |
| ATOM | 2515 | CB | LEU | 156 | 3.910 | 17.699 | 29.605 | 1.00 | 0.00 | −0.106 | 12.77 | 4.00 |
| ATOM | 2516 | HB1 | LEU | 156 | 3.894 | 16.657 | 29.283 | 1.00 | 0.00 | 0.053 | 0.00 | 0.00 |
| ATOM | 2517 | HB2 | LEU | 156 | 4.548 | 17.831 | 30.478 | 1.00 | 0.00 | 0.053 | 0.00 | 0.00 |
| ATOM | 2518 | CG | LEU | 156 | 4.549 | 18.513 | 28.455 | 1.00 | 0.00 | −0.053 | 9.40 | 4.00 |
| ATOM | 2519 | HG | LEU | 156 | 4.574 | 19.573 | 28.706 | 1.00 | 0.00 | 0.053 | 0.00 | 0.00 |
| ATOM | 2520 | CD1 | LEU | 156 | 5.987 | 18.041 | 28.197 | 1.00 | 0.00 | −0.159 | 16.15 | 4.00 |
| ATOM | 2521 | HD1 | LEU | 156 | 6.421 | 18.624 | 27.385 | 1.00 | 0.00 | 0.053 | 0.00 | 0.00 |
| ATOM | 2522 | HD1 | LEU | 156 | 6.582 | 18.177 | 29.100 | 1.00 | 0.00 | 0.053 | 0.00 | 0.00 |
| ATOM | 2523 | HD1 | LEU | 156 | 5.980 | 16.986 | 27.922 | 1.00 | 0.00 | 0.053 | 0.00 | 0.00 |
| ATOM | 2524 | CD2 | LEU | 156 | 3.722 | 18.479 | 27.162 | 1.00 | 0.00 | −0.159 | 16.15 | 4.00 |
| ATOM | 2525 | HD2 | LEU | 156 | 4.223 | 19.068 | 26.394 | 1.00 | 0.00 | 0.053 | 0.00 | 0.00 |
| ATOM | 2526 | HD2 | LEU | 156 | 3.622 | 17.448 | 26.821 | 1.00 | 0.00 | 0.053 | 0.00 | 0.00 |
| ATOM | 2527 | HD2 | LEU | 156 | 2.732 | 18.896 | 27.351 | 1.00 | 0.00 | 0.053 | 0.00 | 0.00 |
| ATOM | 2528 | N | TYR | 157 | 1.087 | 18.718 | 28.136 | 1.00 | 0.00 | −0.650 | 9.00 | −17.40 |
| ATOM | 2529 | HN | TYR | 157 | 1.456 | 19.669 | 28.277 | 1.00 | 0.00 | 0.440 | 0.00 | 0.00 |
| ATOM | 2530 | CA | TYR | 157 | 0.139 | 18.474 | 27.090 | 1.00 | 0.00 | 0.158 | 9.40 | 4.00 |
| ATOM | 2531 | HA | TYR | 157 | −0.572 | 17.732 | 27.452 | 1.00 | 0.00 | 0.053 | 0.00 | 0.00 |
| ATOM | 2532 | C | TYR | 157 | 0.890 | 17.965 | 25.905 | 1.00 | 0.00 | 0.396 | 9.82 | 4.00 |
| ATOM | 2533 | O | TYR | 157 | 0.824 | 18.520 | 24.810 | 1.00 | 0.00 | −0.396 | 8.17 | −17.40 |
| ATOM | 2534 | CB | TYR | 157 | −0.643 | 19.735 | 26.669 | 1.00 | 0.00 | −0.106 | 12.77 | 4.00 |
| ATOM | 2535 | HB1 | TYR | 157 | 0.041 | 20.431 | 26.185 | 1.00 | 0.00 | 0.053 | 0.00 | 0.00 |
| ATOM | 2536 | HB2 | TYR | 157 | −1.077 | 20.195 | 27.556 | 1.00 | 0.00 | 0.053 | 0.00 | 0.00 |
| ATOM | 2537 | CG | TYR | 157 | −1.726 | 19.340 | 25.716 | 1.00 | 0.00 | 0.000 | 7.26 | 0.60 |
| ATOM | 2538 | CD1 | TYR | 157 | −2.848 | 18.686 | 26.174 | 1.00 | 0.00 | −0.127 | 10.80 | 0.60 |
| ATOM | 2539 | HD1 | TYR | 157 | −2.936 | 18.453 | 27.235 | 1.00 | 0.00 | 0.127 | 0.00 | 0.00 |
| ATOM | 2540 | CD2 | TYR | 157 | −1.642 | 19.641 | 24.376 | 1.00 | 0.00 | −0.127 | 10.80 | 0.60 |
| ATOM | 2541 | HD2 | TYR | 157 | −0.768 | 20.170 | 23.994 | 1.00 | 0.00 | 0.127 | 0.00 | 0.00 |
| ATOM | 2542 | CE1 | TYR | 157 | −3.859 | 18.321 | 25.316 | 1.00 | 0.00 | −0.127 | 10.80 | 0.60 |
| ATOM | 2543 | HE1 | TYR | 157 | −4.737 | 17.799 | 25.696 | 1.00 | 0.00 | 0.127 | 0.00 | 0.00 |
| ATOM | 2544 | CE2 | TYR | 157 | −2.651 | 19.279 | 23.513 | 1.00 | 0.00 | −0.127 | 10.80 | 0.60 |
| ATOM | 2545 | HE2 | TYR | 157 | −2.569 | 19.520 | 22.453 | 1.00 | 0.00 | 0.127 | 0.00 | 0.00 |
| ATOM | 2546 | CZ | TYR | 157 | −3.759 | 18.616 | 23.979 | 1.00 | 0.00 | 0.026 | 7.26 | 0.60 |
| ATOM | 2547 | OH | TYR | 157 | −4.792 | 18.245 | 23.092 | 1.00 | 0.00 | −0.451 | 10.94 | −17.40 |
| ATOM | 2548 | HH | TYR | 157 | −5.298 | 19.086 | 22.783 | 1.00 | 0.00 | 0.424 | 0.00 | 0.00 |
| ATOM | 2549 | N | ALA | 158 | 1.642 | 16.872 | 26.123 | 1.00 | 0.00 | −0.650 | 9.00 | −17.40 |
| ATOM | 2550 | HN | ALA | 158 | 1.744 | 16.544 | 27.094 | 1.00 | 0.00 | 0.440 | 0.00 | 0.00 |
| ATOM | 2551 | CA | ALA | 158 | 2.311 | 16.132 | 25.093 | 1.00 | 0.00 | 0.158 | 9.40 | 4.00 |
| ATOM | 2552 | HA | ALA | 158 | 2.748 | 16.801 | 24.352 | 1.00 | 0.00 | 0.053 | 0.00 | 0.00 |
| ATOM | 2553 | C | ALA | 158 | 1.226 | 15.294 | 24.507 | 1.00 | 0.00 | 0.396 | 9.82 | 4.00 |
| ATOM | 2554 | O | ALA | 158 | 1.378 | 14.624 | 23.486 | 1.00 | 0.00 | −0.396 | 8.17 | −17.40 |
| ATOM | 2555 | CB | ALA | 158 | 3.406 | 15.203 | 25.637 | 1.00 | 0.00 | −0.159 | 16.15 | 4.00 |
| ATOM | 2556 | HB1 | ALA | 158 | 3.875 | 14.670 | 24.809 | 1.00 | 0.00 | 0.053 | 0.00 | 0.00 |
| ATOM | 2557 | HB2 | ALA | 158 | 4.157 | 15.793 | 26.161 | 1.00 | 0.00 | 0.053 | 0.00 | 0.00 |
| ATOM | 2558 | HB3 | ALA | 158 | 2.963 | 14.484 | 26.326 | 1.00 | 0.00 | 0.053 | 0.00 | 0.00 |
| ATOM | 2559 | N | MET | 159 | 0.075 | 15.374 | 25.193 | 1.00 | 0.00 | −0.650 | 9.00 | −17.40 |
| ATOM | 2560 | HN | MET | 159 | 0.078 | 16.057 | 25.963 | 1.00 | 0.00 | 0.440 | 0.00 | 0.00 |
| ATOM | 2561 | CA | MET | 159 | −1.153 | 14.666 | 25.028 | 1.00 | 0.00 | 0.158 | 9.40 | 4.00 |
| ATOM | 2562 | HA | MET | 159 | −1.009 | 13.602 | 25.221 | 1.00 | 0.00 | 0.053 | 0.00 | 0.00 |
| ATOM | 2563 | C | MET | 159 | −1.666 | 14.827 | 23.636 | 1.00 | 0.00 | 0.396 | 9.82 | 4.00 |
| ATOM | 2564 | O | MET | 159 | −2.370 | 13.944 | 23.154 | 1.00 | 0.00 | −0.396 | 8.17 | −17.40 |
| ATOM | 2565 | CB | MET | 159 | −2.244 | 15.226 | 25.960 | 1.00 | 0.00 | −0.106 | 12.77 | 4.00 |
| ATOM | 2566 | HB1 | MET | 159 | −3.224 | 14.775 | 25.805 | 1.00 | 0.00 | 0.053 | 0.00 | 0.00 |
| ATOM | 2567 | HB2 | MET | 159 | −2.406 | 16.298 | 25.848 | 1.00 | 0.00 | 0.053 | 0.00 | 0.00 |
| ATOM | 2568 | CG | MET | 159 | −1.944 | 15.023 | 27.449 | 1.00 | 0.00 | −0.041 | 12.77 | 4.00 |
| ATOM | 2569 | HG1 | MET | 159 | −0.949 | 15.420 | 27.650 | 1.00 | 0.00 | 0.053 | 0.00 | 0.00 |
| ATOM | 2570 | HG2 | MET | 159 | −1.984 | 13.954 | 27.659 | 1.00 | 0.00 | 0.053 | 0.00 | 0.00 |
| ATOM | 2571 | SD | MET | 159 | −3.098 | 15.844 | 28.593 | 1.00 | 0.00 | −0.130 | 16.39 | −6.40 |
| ATOM | 2572 | CE | MET | 159 | −2.151 | 17.389 | 28.729 | 1.00 | 0.00 | −0.094 | 16.15 | 4.00 |
| ATOM | 2573 | HE1 | MET | 159 | −2.669 | 18.076 | 29.397 | 1.00 | 0.00 | 0.053 | 0.00 | 0.00 |
| ATOM | 2574 | HE2 | MET | 159 | −1.159 | 17.174 | 29.127 | 1.00 | 0.00 | 0.053 | 0.00 | 0.00 |
| ATOM | 2575 | HE3 | MET | 159 | −2.054 | 17.844 | 27.743 | 1.00 | 0.00 | 0.053 | 0.00 | 0.00 |
| ATOM | 2576 | N | SER | 160 | −1.322 | 15.947 | 22.970 | 1.00 | 0.00 | −0.650 | 9.00 | −17.40 |
| ATOM | 2577 | HN | SER | 160 | −0.640 | 16.574 | 23.420 | 1.00 | 0.00 | 0.440 | 0.00 | 0.00 |
| ATOM | 2578 | CA | SER | 160 | −1.828 | 16.339 | 21.680 | 1.00 | 0.00 | 0.158 | 9.40 | 4.00 |
| ATOM | 2579 | HA | SER | 160 | −2.807 | 16.810 | 21.756 | 1.00 | 0.00 | 0.053 | 0.00 | 0.00 |
| ATOM | 2580 | C | SER | 160 | −1.974 | 15.167 | 20.763 | 1.00 | 0.00 | 0.396 | 9.82 | 4.00 |
| ATOM | 2581 | O | SER | 160 | −3.070 | 14.897 | 20.285 | 1.00 | 0.00 | −0.396 | 8.17 | −17.40 |
| ATOM | 2582 | CB | SER | 160 | −0.883 | 17.315 | 20.956 | 1.00 | 0.00 | 0.007 | 12.77 | 4.00 |
| ATOM | 2583 | HB1 | SER | 160 | −1.296 | 17.616 | 19.993 | 1.00 | 0.00 | 0.053 | 0.00 | 0.00 |
| ATOM | 2584 | HB2 | SER | 160 | 0.088 | 16.855 | 20.775 | 1.00 | 0.00 | 0.053 | 0.00 | 0.00 |
| ATOM | 2585 | OG | SER | 160 | −0.675 | 18.487 | 21.728 | 1.00 | 0.00 | −0.537 | 11.04 | −17.40 |
| ATOM | 2586 | HG | SER | 160 | 0.177 | 18.965 | 21.403 | 1.00 | 0.00 | 0.424 | 0.00 | 0.00 |
| ATOM | 2587 | N | ALA | 161 | −0.900 | 14.404 | 20.514 | 1.00 | 0.00 | −0.650 | 9.00 | −17.40 |
| ATOM | 2588 | HN | ALA | 161 | −0.007 | 14.584 | 20.994 | 1.00 | 0.00 | 0.440 | 0.00 | 0.00 |
| ATOM | 2589 | CA | ALA | 161 | −1.015 | 13.332 | 19.566 | 1.00 | 0.00 | 0.158 | 9.40 | 4.00 |
| ATOM | 2590 | HA | ALA | 161 | −1.280 | 13.716 | 18.581 | 1.00 | 0.00 | 0.053 | 0.00 | 0.00 |
| ATOM | 2591 | C | ALA | 161 | −2.074 | 12.361 | 19.992 | 1.00 | 0.00 | 0.396 | 9.82 | 4.00 |
| ATOM | 2592 | O | ALA | 161 | −2.184 | 12.021 | 21.168 | 1.00 | 0.00 | −0.396 | 8.17 | −17.40 |
| ATOM | 2593 | CB | ALA | 161 | 0.288 | 12.538 | 19.378 | 1.00 | 0.00 | −0.159 | 16.15 | 4.00 |
| ATOM | 2594 | HB1 | ALA | 161 | 0.128 | 11.744 | 18.648 | 1.00 | 0.00 | 0.053 | 0.00 | 0.00 |
| ATOM | 2595 | HB2 | ALA | 161 | 1.072 | 13.205 | 19.022 | 1.00 | 0.00 | 0.053 | 0.00 | 0.00 |
| ATOM | 2596 | HB3 | ALA | 161 | 0.588 | 12.100 | 20.330 | 1.00 | 0.00 | 0.053 | 0.00 | 0.00 |
| ATOM | 2597 | N | ASN | 162 | −2.872 | 11.884 | 19.009 | 1.00 | 0.00 | −0.650 | 9.00 | −17.40 |
| ATOM | 2598 | HN | ASN | 162 | −2.725 | 12.242 | 18.054 | 1.00 | 0.00 | 0.440 | 0.00 | 0.00 |
| ATOM | 2599 | CA | ASN | 162 | −3.912 | 10.909 | 19.207 | 1.00 | 0.00 | 0.158 | 9.40 | 4.00 |
| ATOM | 2600 | HA | ASN | 162 | −4.626 | 10.899 | 18.384 | 1.00 | 0.00 | 0.053 | 0.00 | 0.00 |
| ATOM | 2601 | C | ASN | 162 | −4.693 | 11.167 | 20.451 | 1.00 | 0.00 | 0.396 | 9.82 | 4.00 |
| ATOM | 2602 | O | ASN | 162 | −4.340 | 10.693 | 21.528 | 1.00 | 0.00 | −0.396 | 8.17 | −17.40 |
| ATOM | 2603 | CB | ASN | 162 | −3.374 | 9.467 | 19.268 | 1.00 | 0.00 | −0.106 | 12.77 | 4.00 |
| ATOM | 2604 | HB1 | ASN | 162 | −2.652 | 9.391 | 20.081 | 1.00 | 0.00 | 0.053 | 0.00 | 0.00 |
| ATOM | 2605 | HB2 | ASN | 162 | −2.892 | 9.227 | 18.320 | 1.00 | 0.00 | 0.053 | 0.00 | 0.00 |
| ATOM | 2606 | CG | ASN | 162 | −4.535 | 8.514 | 19.515 | 1.00 | 0.00 | 0.396 | 9.82 | 4.00 |
| ATOM | 2607 | OD1 | ASN | 162 | −4.927 | 8.312 | 20.663 | 1.00 | 0.00 | −0.396 | 8.17 | −17.40 |
| ATOM | 2608 | ND2 | ASN | 162 | −5.090 | 7.913 | 18.428 | 1.00 | 0.00 | −0.879 | 13.25 | 17.40 |
| ATOM | 2609 | HD2 | ASN | 162 | −4.725 | 8.115 | 17.485 | 1.00 | 0.00 | 0.440 | 0.00 | 0.00 |
| ATOM | 2610 | HD2 | ASN | 162 | −5.874 | 7.255 | 18.547 | 1.00 | 0.00 | 0.440 | 0.00 | 0.00 |
| ATOM | 2611 | N | TRP | 163 | −5.781 | 11.954 | 20.352 | 1.00 | 0.00 | −0.650 | 9.00 | −17.40 |
| ATOM | 2612 | HN | TRP | 163 | −6.063 | 12.381 | 19.458 | 1.00 | 0.00 | 0.440 | 0.00 | 0.00 |
| ATOM | 2613 | CA | TRP | 163 | −6.523 | 12.164 | 21.555 | 1.00 | 0.00 | 0.158 | 9.40 | 4.00 |
| ATOM | 2614 | HA | TRP | 163 | −6.299 | 11.365 | 22.262 | 1.00 | 0.00 | 0.053 | 0.00 | 0.00 |
| ATOM | 2615 | C | TRP | 163 | −7.984 | 12.161 | 21.243 | 1.00 | 0.00 | 0.396 | 9.82 | 4.00 |
| ATOM | 2616 | O | TRP | 163 | −8.405 | 12.592 | 20.171 | 1.00 | 0.00 | −0.396 | 8.17 | −17.40 |
| ATOM | 2617 | CB | TRP | 163 | −6.124 | 13.466 | 22.273 | 1.00 | 0.00 | −0.106 | 12.77 | 4.00 |
| ATOM | 2618 | HB1 | TRP | 163 | −5.060 | 13.113 | 22.504 | 1.00 | 0.00 | 0.053 | 0.00 | 0.00 |
| ATOM | 2619 | HB2 | TRP | 163 | −6.714 | 13.144 | 23.185 | 1.00 | 0.00 | 0.053 | 0.00 | 0.00 |
| ATOM | 2620 | CG | TRP | 163 | −6.352 | 14.725 | 21.478 | 1.00 | 0.00 | 0.000 | 7.26 | 0.60 |
| ATOM | 2621 | CD1 | TRP | 163 | −5.788 | 15.106 | 20.299 | 1.00 | 0.00 | −0.177 | 10.80 | 0.60 |
| ATOM | 2622 | HD1 | TRP | 163 | −5.120 | 14.488 | 19.698 | 1.00 | 0.00 | 0.127 | 0.00 | 0.00 |
| ATOM | 2623 | CD2 | TRP | 163 | −7.145 | 15.834 | 21.925 | 1.00 | 0.00 | 0.000 | 6.80 | 0.60 |
| ATOM | 2624 | NE1 | TRP | 163 | −6.181 | 16.380 | 19.976 | 1.00 | 0.00 | −0.292 | 9.00 | −17.40 |
| ATOM | 2625 | HE1 | TRP | 163 | −5.901 | 16.902 | 19.133 | 1.00 | 0.00 | 0.393 | 0.00 | 0.00 |
| ATOM | 2626 | CE2 | TRP | 163 | −7.014 | 16.842 | 20.971 | 1.00 | 0.00 | −0.050 | 6.80 | 0.60 |
| ATOM | 2627 | CE3 | TRP | 163 | −7.903 | 16.005 | 23.048 | 1.00 | 0.00 | −0.127 | 10.80 | 0.60 |
| ATOM | 2628 | HE3 | TRP | 163 | −7.990 | 15.219 | 23.798 | 1.00 | 0.00 | 0.127 | 0.00 | 0.00 |
| ATOM | 2629 | CZ2 | TRP | 163 | −7.644 | 18.043 | 21.126 | 1.00 | 0.00 | −0.127 | 10.80 | 0.60 |
| ATOM | 2630 | HZ2 | TRP | 163 | −7.534 | 18.839 | 20.390 | 1.00 | 0.00 | 0.127 | 0.00 | 0.00 |
| ATOM | 2631 | CZ3 | TRP | 163 | −8.552 | 17.210 | 23.193 | 1.00 | 0.00 | −0.127 | 10.80 | 0.60 |
| ATOM | 2632 | HZ3 | TRP | 163 | −9.178 | 17.378 | 24.068 | 1.00 | 0.00 | 0.127 | 0.00 | 0.00 |
| ATOM | 2633 | CH2 | TRP | 163 | −8.423 | 18.208 | 22.249 | 1.00 | 0.00 | −0.127 | 10.80 | 0.60 |
| ATOM | 2634 | HH2 | TRP | 163 | −8.950 | 19.150 | 22.396 | 1.00 | 0.00 | 0.127 | 0.00 | 0.00 |
| ATOM | 2635 | N | LYS | 164S | −8.794 | 11.631 | 22.185 | 1.00 | 0.00 | −0.650 | 9.00 | −17.40 |
| ATOM | 2636 | HN | LYS | 164S | −8.375 | 11.239 | 23.040 | 1.00 | 0.00 | 0.440 | 0.00 | 0.00 |
| ATOM | 2637 | CA | LYS | 164S | −10.216 | 11.596 | 22.031 | 1.00 | 0.00 | 0.158 | 9.40 | 4.00 |
| ATOM | 2638 | HA | LYS | 164S | −10.422 | 11.748 | 20.971 | 1.00 | 0.00 | 0.053 | 0.00 | 0.00 |
| ATOM | 2639 | C | LYS | 164S | −10.767 | 12.695 | 22.870 | 1.00 | 0.00 | 0.396 | 9.82 | 4.00 |
| ATOM | 2640 | O | LYS | 164S | −10.240 | 13.013 | 23.935 | 1.00 | 0.00 | −0.396 | 8.17 | −17.40 |
| ATOM | 2641 | CB | LYS | 164S | −10.863 | 10.265 | 22.457 | 1.00 | 0.00 | −0.106 | 12.77 | 4.00 |
| ATOM | 2642 | HB1 | LYS | 164S | −10.530 | 9.488 | 21.768 | 1.00 | 0.00 | 0.053 | 0.00 | 0.00 |
| ATOM | 2643 | HB2 | LYS | 164S | −11.945 | 10.381 | 22.411 | 1.00 | 0.00 | 0.053 | 0.00 | 0.00 |
| ATOM | 2644 | CG | LYS | 164S | −10.505 | 9.812 | 23.874 | 1.00 | 0.00 | −0.106 | 12.77 | 4.00 |
| ATOM | 2645 | HG1 | LYS | 164S | −11.025 | 8.906 | 24.187 | 1.00 | 0.00 | 0.053 | 0.00 | 0.00 |
| ATOM | 2646 | HG2 | LYS | 164S | −10.737 | 10.551 | 24.640 | 1.00 | 0.00 | 0.053 | 0.00 | 0.00 |
| ATOM | 2647 | CD | LYS | 164S | −9.019 | 9.501 | 24.060 | 1.00 | 0.00 | −0.106 | 12.77 | 4.00 |
| ATOM | 2648 | HD1 | LYS | 164S | −8.370 | 10.365 | 23.914 | 1.00 | 0.00 | 0.053 | 0.00 | 0.00 |
| ATOM | 2649 | HD2 | LYS | 164S | −8.642 | 8.744 | 23.371 | 1.00 | 0.00 | 0.053 | 0.00 | 0.00 |
| ATOM | 2650 | CE | LYS | 164S | −8.673 | 8.976 | 25.455 | 1.00 | 0.00 | 0.099 | 12.77 | 4.00 |
| ATOM | 2651 | HE1 | LYS | 164S | −9.315 | 8.130 | 25.702 | 1.00 | 0.00 | 0.053 | 0.00 | 0.00 |
| ATOM | 2652 | HE2 | LYS | 164S | −8.820 | 9.763 | 26.194 | 1.00 | 0.00 | 0.053 | 0.00 | 0.00 |
| ATOM | 2653 | NZ | LYS | 164S | −7.260 | 8.537 | 25.491 | 1.00 | 0.00 | −0.045 | 13.25 | −39.20 |
| ATOM | 2654 | HZ1 | LYS | 164S | −7.033 | 8.184 | 26.432 | 1.00 | 0.00 | 0.280 | 0.00 | 0.00 |
| ATOM | 2655 | HZ2 | LYS | 164S | −7.115 | 7.786 | 24.800 | 1.00 | 0.00 | 0.280 | 0.00 | 0.00 |
| ATOM | 2656 | HZ3 | LYS | 164S | −6.646 | 9.333 | 25.265 | 1.00 | 0.00 | 0.280 | 0.00 | 0.00 |
| ATOM | 2657 | N | TYR | 165 | −11.848 | 13.324 | 22.377 | 1.00 | 0.00 | −0.650 | 9.00 | −17.40 |
| ATOM | 2658 | HN | TYR | 165 | −12.299 | 12.962 | 21.525 | 1.00 | 0.00 | 0.440 | 0.00 | 0.00 |
| ATOM | 2659 | CA | TYR | 165 | −12.380 | 14.485 | 23.015 | 1.00 | 0.00 | 0.158 | 9.40 | 4.00 |
| ATOM | 2660 | HA | TYR | 165 | −11.937 | 14.496 | 24.010 | 1.00 | 0.00 | 0.053 | 0.00 | 0.00 |
| ATOM | 2661 | C | TYR | 165 | −13.858 | 14.286 | 23.031 | 1.00 | 0.00 | 0.396 | 9.82 | 4.00 |
| ATOM | 2662 | O | TYR | 165 | −14.420 | 13.684 | 22.116 | 1.00 | 0.00 | −0.396 | 8.17 | −17.40 |
| ATOM | 2663 | CB | TYR | 165 | −11.990 | 15.725 | 22.181 | 1.00 | 0.00 | −0.106 | 12.77 | 4.00 |
| ATOM | 2664 | HB1 | TYR | 165 | −12.612 | 15.742 | 21.286 | 1.00 | 0.00 | 0.053 | 0.00 | 0.00 |
| ATOM | 2665 | HB2 | TYR | 165 | −10.936 | 15.639 | 21.916 | 1.00 | 0.00 | 0.053 | 0.00 | 0.00 |
| ATOM | 2666 | CG | TYR | 165 | −12.173 | 17.031 | 22.882 | 1.00 | 0.00 | 0.000 | 7.26 | 0.60 |
| ATOM | 2667 | CD1 | TYR | 165 | −11.515 | 17.302 | 24.062 | 1.00 | 0.00 | −0.127 | 10.80 | 0.60 |
| ATOM | 2668 | HD1 | TYR | 165 | −10.869 | 16.542 | 24.504 | 1.00 | 0.00 | 0.127 | 0.00 | 0.00 |
| ATOM | 2669 | CD2 | TYR | 165 | −12.957 | 18.011 | 22.316 | 1.00 | 0.00 | −0.127 | 10.80 | 0.60 |
| ATOM | 2670 | HD2 | TYR | 165 | −13.462 | 17.822 | 21.368 | 1.00 | 0.00 | 0.127 | 0.00 | 0.00 |
| ATOM | 2671 | CE1 | TYP. | 165 | −11.661 | 18.516 | 24.689 | 1.00 | 0.00 | −0.127 | 10.80 | 0.60 |
| ATOM | 2672 | HE1 | TYR | 165 | −11.147 | 18.713 | 25.629 | 1.00 | 0.00 | 0.127 | 0.00 | 0.00 |
| ATOM | 2673 | CE2 | TYR | 165 | −13.105 | 19.225 | 22.939 | 1.00 | 0.00 | −0.127 | 10.80 | 0.60 |
| ATOM | 2674 | HE2 | TYR | 165 | −13.738 | 19.990 | 22.492 | 1.00 | 0.00 | 0.127 | 0.00 | 0.00 |
| ATOM | 2675 | CZ | TYR | 165 | −12.459 | 19.478 | 24.121 | 1.00 | 0.00 | 0.026 | 7.26 | 0.60 |
| ATOM | 2676 | OH | TYR | 165 | −12.615 | 20.730 | 24.748 | 1.00 | 0.00 | −0.451 | 10.94 | −17.40 |
| ATOM | 2677 | HH | TYR | 165 | −11.790 | 21.316 | 24.555 | 1.00 | 0.00 | 0.424 | 0.00 | 0.00 |
| ATOM | 2678 | N | LEU | 166 | −14.526 | 14.746 | 24.103 | 1.00 | 0.00 | −0.650 | 9.00 | −17.40 |
| ATOM | 2679 | HN | LEU | 166 | −14.019 | 15.228 | 24.859 | 1.00 | 0.00 | 0.440 | 0.00 | 0.00 |
| ATOM | 2680 | CA | LEU | 166 | −15.944 | 14.563 | 24.191 | 1.00 | 0.00 | 0.158 | 9.40 | 4.00 |
| ATOM | 2681 | HA | LEU | 166 | −16.228 | 13.683 | 23.614 | 1.00 | 0.00 | 0.053 | 0.00 | 0.00 |
| ATOM | 2682 | C | LEU | 166 | −16.629 | 15.768 | 23.642 | 1.00 | 0.00 | 0.396 | 9.82 | 4.00 |
| ATOM | 2683 | O | LEU | 166 | −16.036 | 16.826 | 23.436 | 1.00 | 0.00 | −0.396 | 8.17 | −17.40 |
| ATOM | 2684 | CB | LEU | 166 | −16.483 | 14.342 | 25.626 | 1.00 | 0.00 | −0.106 | 12.77 | 4.00 |
| ATOM | 2685 | HB1 | LEU | 166 | −17.553 | 14.549 | 25.615 | 1.00 | 0.00 | 0.053 | 0.00 | 0.00 |
| ATOM | 2686 | HB2 | LEU | 166 | −15.956 | 15.029 | 26.288 | 1.00 | 0.00 | 0.053 | 0.00 | 0.00 |
| ATOM | 2687 | CG | LEU | 166 | −16.305 | 12.922 | 26.216 | 1.00 | 0.00 | −0.053 | 9.40 | 4.00 |
| ATOM | 2688 | HG | LEU | 166 | −16.457 | 12.922 | 27.295 | 1.00 | 0.00 | 0.053 | 0.00 | 0.00 |
| ATOM | 2689 | CD1 | LEU | 166 | −17.309 | 11.938 | 25.602 | 1.00 | 0.00 | −0.159 | 16.15 | 4.00 |
| ATOM | 2690 | HD1 | LEU | 166 | −17.160 | 10.948 | 26.035 | 1.00 | 0.00 | 0.053 | 0.00 | 0.00 |
| ATOM | 2691 | HD1 | LEU | 166 | −18.324 | 12.276 | 25.810 | 1.00 | 0.00 | 0.053 | 0.00 | 0.00 |
| ATOM | 2692 | HD1 | LEU | 166 | −17.157 | 11.888 | 24.523 | 1.00 | 0.00 | 0.053 | 0.00 | 0.00 |
| ATOM | 2693 | CD2 | LEU | 166 | −14.865 | 12.413 | 26.085 | 1.00 | 0.00 | −0.159 | 16.15 | 4.00 |
| ATOM | 2694 | HD2 | LEU | 166 | −14.792 | 11.413 | 26.513 | 1.00 | 0.00 | 0.053 | 0.00 | 0.00 |
| ATOM | 2695 | HD2 | LEU | 166 | −14.586 | 12.377 | 25.031 | 1.00 | 0.00 | 0.053 | 0.00 | 0.00 |
| ATOM | 2696 | HD2 | LEU | 166 | −14.191 | 13.085 | 26.616 | 1.00 | 0.00 | 0.053 | 0.00 | 0.00 |
| ATOM | 2697 | N | ILE | 167 | −17.928 | 15.580 | 23.363 | 1.00 | 0.00 | −0.650 | 9.00 | −17.40 |
| ATOM | 2698 | HN | ILE | 167 | −18.310 | 14.637 | 23.524 | 1.00 | 0.00 | 0.440 | 0.00 | 0.00 |
| ATOM | 2699 | CA | ILE | 167 | −18.827 | 16.569 | 22.863 | 1.00 | 0.00 | 0.158 | 9.40 | 4.00 |
| ATOM | 2700 | HA | ILE | 167 | −18.387 | 16.970 | 21.949 | 1.00 | 0.00 | 0.053 | 0.00 | 0.00 |
| ATOM | 2701 | C | ILE | 167 | −18.960 | 17.619 | 23.921 | 1.00 | 0.00 | 0.396 | 9.82 | 4.00 |
| ATOM | 2702 | O | ILE | 167 | −19.043 | 18.809 | 23.621 | 1.00 | 0.00 | −0.396 | 8.17 | −17.40 |
| ATOM | 2703 | CB | ILE | 167 | −20.186 | 15.964 | 22.635 | 1.00 | 0.00 | −0.053 | 9.40 | 4.00 |
| ATOM | 2704 | HB | ILE | 167 | −20.089 | 15.226 | 21.838 | 1.00 | 0.00 | 0.053 | 0.00 | 0.00 |
| ATOM | 2705 | CG1 | ILE | 167 | −21.186 | 16.987 | 22.077 | 1.00 | 0.00 | −0.106 | 12.77 | 4.00 |
| ATOM | 2706 | HG1 | ILE | 167 | −20.787 | 17.543 | 21.228 | 1.00 | 0.00 | 0.053 | 0.00 | 0.00 |
| ATOM | 2707 | HG1 | ILE | 167 | −21.480 | 17.731 | 22.816 | 1.00 | 0.00 | 0.053 | 0.00 | 0.00 |
| ATOM | 2708 | CG2 | ILE | 167 | −20.630 | 15.306 | 23.952 | 1.00 | 0.00 | −0.159 | 16.15 | 4.00 |
| ATOM | 2709 | HG2 | ILE | 167 | −21.614 | 14.856 | 23.820 | 1.00 | 0.00 | 0.053 | 0.00 | 0.00 |
| ATOM | 2710 | HG2 | ILE | 167 | −19.913 | 14.534 | 24.232 | 1.00 | 0.00 | 0.053 | 0.00 | 0.00 |
| ATOM | 2711 | HG2 | ILE | 167 | −20.677 | 16.060 | 24.737 | 1.00 | 0.00 | 0.053 | 0.00 | 0.00 |
| ATOM | 2712 | CD1 | ILE | 167 | −22.480 | 16.338 | 21.589 | 1.00 | 0.00 | −0.159 | 16.15 | 4.00 |
| ATOM | 2713 | HD1 | ILE | 167 | −23.150 | 17.106 | 21.205 | 1.00 | 0.00 | 0.053 | 0.00 | 0.00 |
| ATOM | 2714 | HD1 | ILE | 167 | −22.253 | 15.625 | 20.795 | 1.00 | 0.00 | 0.053 | 0.00 | 0.00 |
| ATOM | 2715 | HD1 | ILE | 167 | −22.961 | 15.817 | 22.417 | 1.00 | 0.00 | 0.053 | 0.00 | 0.00 |
| ATOM | 2716 | N | ASN | 168 | −18.937 | 17.191 | 25.197 | 1.00 | 0.00 | −0.650 | 9.00 | −17.40 |
| ATOM | 2717 | HN | ASN | 168 | −18.717 | 16.201 | 25.379 | 1.00 | 0.00 | 0.440 | 0.00 | 0.00 |
| ATOM | 2718 | CA | ASN | 168 | −19.204 | 18.051 | 26.314 | 1.00 | 0.00 | 0.158 | 9.40 | 4.00 |
| ATOM | 2719 | HA | ASN | 168 | −20.201 | 18.490 | 26.290 | 1.00 | 0.00 | 0.053 | 0.00 | 0.00 |
| ATOM | 2720 | C | ASN | 168 | −18.263 | 19.207 | 26.407 | 1.00 | 0.00 | 0.396 | 9.82 | 4.00 |
| ATOM | 2721 | O | ASN | 168 | −18.725 | 20.335 | 26.576 | 1.00 | 0.00 | −0.396 | 8.17 | −17.40 |
| ATOM | 2722 | CB | ASN | 168 | −19.123 | 17.334 | 27.677 | 1.00 | 0.00 | −0.106 | 12.77 | 4.00 |
| ATOM | 2723 | HB1 | ASN | 168 | −19.064 | 18.070 | 28.478 | 1.00 | 0.00 | 0.053 | 0.00 | 0.00 |
| ATOM | 2724 | HB2 | ASN | 168 | −18.236 | 16.700 | 27.708 | 1.00 | 0.00 | 0.053 | 0.00 | 0.00 |
| ATOM | 2725 | CG | ASN | 168 | −20.362 | 16.471 | 27.880 | 1.00 | 0.00 | 0.396 | 9.82 | 4.00 |
| ATOM | 2726 | OD1 | ASN | 168 | −21.484 | 16.915 | 27.640 | 1.00 | 0.00 | −0.396 | 8.17 | −17.40 |
| ATOM | 2727 | ND2 | ASN | 168 | −20.162 | 15.208 | 28.343 | 1.00 | 0.00 | −0.879 | 13.25 | −17.40 |
| ATOM | 2728 | HD2 | ASN | 168 | −19.206 | 14.874 | 28.531 | 1.00 | 0.00 | 0.440 | 0.00 | 0.00 |
| ATOM | 2729 | HD2 | ASN | 168 | −20.966 | 14.585 | 28.505 | 1.00 | 0.00 | 0.440 | 0.00 | 0.00 |
| ATOM | 2730 | N | LEU | 169 | −16.934 | 18.989 | 26.299 | 1.00 | 0.00 | −0.650 | 9.00 | −17.40 |
| ATOM | 2731 | HN | LEU | 169 | −16.559 | 18.065 | 26.037 | 1.00 | 0.00 | 0.440 | 0.00 | 0.00 |
| ATOM | 2732 | CA | LEU | 169 | −16.066 | 20.104 | 26.566 | 1.00 | 0.00 | 0.158 | 9.40 | 4.00 |
| ATOM | 2733 | HA | LEU | 169 | −16.211 | 20.479 | 27.578 | 1.00 | 0.00 | 0.053 | 0.00 | 0.00 |
| ATOM | 2734 | C | LEU | 169 | −16.329 | 21.226 | 25.621 | 1.00 | 0.00 | 0.396 | 9.82 | 4.00 |
| ATOM | 2735 | O | LEU | 169 | −16.756 | 22.296 | 26.050 | 1.00 | 0.00 | −0.396 | 8.17 | −17.40 |
| ATOM | 2736 | CB | LEU | 169 | −14.570 | 19.754 | 26.521 | 1.00 | 0.00 | −0.106 | 12.77 | 4.00 |
| ATOM | 2737 | HB1 | LEU | 169 | −13.944 | 20.646 | 26.543 | 1.00 | 0.00 | 0.053 | 0.00 | 0.00 |
| ATOM | 2738 | HB2 | LEU | 169 | −14.309 | 19.203 | 25.616 | 1.00 | 0.00 | 0.053 | 0.00 | 0.00 |
| ATOM | 2739 | CG | LEU | 169 | −14.151 | 18.881 | 27.715 | 1.00 | 0.00 | −0.053 | 9.40 | 4.00 |
| ATOM | 2740 | HG | LEU | 169 | −14.635 | 17.904 | 27.693 | 1.00 | 0.00 | 0.053 | 0.00 | 0.00 |
| ATOM | 2741 | CD1 | LEU | 169 | −12.637 | 18.631 | 27.736 | 1.00 | 0.00 | −0.159 | 16.15 | 4.00 |
| ATOM | 2742 | HD1 | LEU | 169 | −12.382 | 18.010 | 28.595 | 1.00 | 0.00 | 0.053 | 0.00 | 0.00 |
| ATOM | 2743 | HD1 | LEU | 169 | −12.339 | 18.121 | 26.819 | 1.00 | 0.00 | 0.053 | 0.00 | 0.00 |
| ATOM | 2744 | HD1 | LEU | 169 | −12.111 | 19.583 | 27.808 | 1.00 | 0.00 | 0.053 | 0.00 | 0.00 |
| ATOM | 2745 | CD2 | LEU | 169 | −14.677 | 19.480 | 29.027 | 1.00 | 0.00 | −0.159 | 16.15 | 4.00 |
| ATOM | 2746 | HD2 | LEU | 169 | −14.371 | 18.850 | 29.862 | 1.00 | 0.00 | 0.053 | 0.00 | 0.00 |
| ATOM | 2747 | HD2 | LEU | 169 | −14.268 | 20.481 | 29.159 | 1.00 | 0.00 | 0.053 | 0.00 | 0.00 |
| ATOM | 2748 | HD2 | LEU | 169 | −15.765 | 19.534 | 28.991 | 1.00 | 0.00 | 0.053 | 0.00 | 0.00 |
| ATOM | 2749 | N | CYS | 170S | −16.149 | 20.990 | 24.307 | 1.00 | 0.00 | −0.65 | 9.00 | −17.40 |
| ATOM | 2750 | HN | CYS | 170S | −15.826 | 20.052 | 24.027 | 1.00 | 0.00 | 0.44 | 0.00 | 0.00 |
| ATOM | 2751 | CA | CYS | 170S | −16.376 | 21.959 | 23.265 | 1.00 | 0.00 | 0.15 | 9.40 | 4.00 |
| ATOM | 2752 | HA | CYS | 170S | −15.908 | 21.666 | 22.324 | 1.00 | 0.00 | 0.05 | 0.00 | 0.00 |
| ATOM | 2753 | C | CYS | 170S | −15.820 | 23.321 | 23.623 | 1.00 | 0.00 | 0.39 | 9.82 | 4.00 |
| ATOM | 2754 | O | CYS | 170S | −16.194 | 24.320 | 23.012 | 1.00 | 0.00 | −0.39 | 8.17 | −17.40 |
| ATOM | 2755 | CB | CYS | 170S | −17.882 | 22.085 | 22.955 | 1.00 | 0.00 | −0.04 | 12.77 | 4.00 |
| ATOM | 2756 | HB1 | CYS | 170S | −18.366 | 22.571 | 23.801 | 1.00 | 0.00 | 0.05 | 0.00 | 0.00 |
| ATOM | 2757 | HB2 | CYS | 170S | −18.286 | 21.083 | 22.804 | 1.00 | 0.00 | 0.05 | 0.00 | 0.00 |
| ATOM | 2758 | SG | CYS | 170S | −18.255 | 23.060 | 21.470 | 1.00 | 0.00 | −0.25 | 19.93 | 6.40 |
| ATOM | 2759 | HG | CYS | 170S | −17.668 | 24.261 | 21.564 | 1.00 | 0.00 | 0.19 | 0.00 | 0.00 |
| ATOM | 2760 | N | GLY | 171 | −14.901 | 23.406 | 24.606 | 1.00 | 0.00 | −0.650 | 9.00 | −17.40 |
| ATOM | 2761 | HN | GLY | 171 | −14.580 | 22.536 | 25.054 | 1.00 | 0.00 | 0.440 | 0.00 | 0.00 |
| ATOM | 2762 | CA | GLY | 171 | −14.350 | 24.650 | 25.056 | 1.00 | 0.00 | 0.105 | 9.40 | 4.00 |
| ATOM | 2763 | HA1 | GLY | 171 | −13.852 | 24.511 | 26.016 | 1.00 | 0.00 | 0.053 | 0.00 | 0.00 |
| ATOM | 2764 | HA2 | GLY | 171 | −15.143 | 25.388 | 25.171 | 1.00 | 0.00 | 0.053 | 0.00 | 0.00 |
| ATOM | 2765 | C | GLY | 171 | −13.358 | 25.155 | 24.065 | 1.00 | 0.00 | 0.396 | 9.82 | 4.00 |
| ATOM | 2766 | O | GLY | 171 | −13.200 | 26.360 | 23.877 | 1.00 | 0.00 | −0.396 | 8.17 | −17.40 |
| ATOM | 2767 | N | MET | 172 | −12.659 | 24.221 | 23.404 | 1.00 | 0.00 | −0.650 | 9.00 | −17.40 |
| ATOM | 2768 | HN | MET | 172 | −12.912 | 23.229 | 23.521 | 1.00 | 0.00 | 0.440 | 0.00 | 0.00 |
| ATOM | 2769 | CA | MET | 172 | −11.570 | 24.563 | 22.540 | 1.00 | 0.00 | 0.158 | 9.40 | 4.00 |
| ATOM | 2770 | HA | MET | 172 | −10.842 | 25.185 | 23.060 | 1.00 | 0.00 | 0.053 | 0.00 | 0.00 |
| ATOM | 2771 | C | MET | 172 | −12.054 | 25.317 | 21.348 | 1.00 | 0.00 | 0.396 | 9.82 | 4.00 |
| ATOM | 2772 | O | MET | 172 | −11.242 | 25.901 | 20.632 | 1.00 | 0.00 | −0.396 | 8.17 | −17.40 |
| ATOM | 2773 | CB | MET | 172 | −10.732 | 23.369 | 22.054 | 1.00 | 0.00 | −0.106 | 12.77 | 4.00 |
| ATOM | 2774 | HB1 | MET | 172 | −11.247 | 22.819 | 21.266 | 1.00 | 0.00 | 0.053 | 0.00 | 0.00 |
| ATOM | 2775 | HB2 | MET | 172 | −10.531 | 22.671 | 22.866 | 1.00 | 0.00 | 0.053 | 0.00 | 0.00 |
| ATOM | 2776 | CG | MET | 172 | −9.383 | 23.830 | 21.493 | 1.00 | 0.00 | −0.041 | 12.77 | 4.00 |
| ATOM | 2777 | HG1 | MET | 172 | −8.918 | 24.485 | 22.229 | 1.00 | 0.00 | 0.053 | 0.00 | 0.00 |
| ATOM | 2778 | HG2 | MET | 172 | −9.570 | 24.362 | 20.560 | 1.00 | 0.00 | 0.053 | 0.00 | 0.00 |
| ATOM | 2779 | SD | MET | 172 | −8.192 | 22.508 | 21.125 | 1.00 | 0.00 | −0.130 | 16.39 | −6.40 |
| ATOM | 2780 | CE | MET | 172 | −9.038 | 21.971 | 19.612 | 1.00 | 0.00 | −0.094 | 16.15 | 4.00 |
| ATOM | 2781 | HE1 | MET | 172 | −8.494 | 21.137 | 19.167 | 1.00 | 0.00 | 0.053 | 0.00 | 0.00 |
| ATOM | 2782 | HE2 | MET | 172 | −10.052 | 21.654 | 19.855 | 1.00 | 0.00 | 0.053 | 0.00 | 0.00 |
| ATOM | 2783 | HE3 | MET | 172 | −9.076 | 22.798 | 18.903 | 1.00 | 0.00 | 0.053 | 0.00 | 0.00 |
| ATOM | 2784 | N | ASP | 173P | −13.382 | 25.324 | 21.113 | 1.00 | 0.00 | −0.650 | 9.00 | −17.40 |
| ATOM | 2785 | HN | ASP | 173P | −14.003 | 24.872 | 21.798 | 1.00 | 0.00 | 0.440 | 0.00 | 0.00 |
| ATOM | 2786 | CA | ASP | 173P | −13.971 | 25.932 | 19.951 | 1.00 | 0.00 | 0.158 | 9.40 | 4.00 |
| ATOM | 2787 | HA | ASP | 173P | −13.770 | 25.330 | 19.064 | 1.00 | 0.00 | 0.053 | 0.00 | 0.00 |
| ATOM | 2788 | C | ASP | 173P | −13.406 | 27.300 | 19.735 | 1.00 | 0.00 | 0.396 | 9.82 | 4.00 |
| ATOM | 2789 | O | ASP | 173P | −13.226 | 27.693 | 18.584 | 1.00 | 0.00 | −0.396 | 8.17 | −17.40 |
| ATOM | 2790 | CB | ASP | 173P | −15.498 | 26.087 | 20.039 | 1.00 | 0.00 | −0.336 | 12.77 | 4.00 |
| ATOM | 2791 | HB1 | ASP | 173P | −15.819 | 26.766 | 19.249 | 1.00 | 0.00 | 0.053 | 0.00 | 0.00 |
| ATOM | 2792 | HB2 | ASP | 173P | −15.744 | 26.494 | 21.019 | 1.00 | 0.00 | 0.053 | 0.00 | 0.00 |
| ATOM | 2793 | CG | ASP | 173P | −16.128 | 24.717 | 19.855 | 1.00 | 0.00 | 0.297 | 9.82 | 4.00 |
| ATOM | 2794 | OD1 | ASP | 173P | −15.487 | 23.703 | 20.243 | 1.00 | 0.00 | −0.534 | 6.17 | −18.95 |
| ATOM | 2795 | OD2 | ASP | 173P | −17.268 | 24.673 | 19.320 | 1.00 | 0.00 | −0.534 | 8.17 | −18.95 |
| ATOM | 2796 | N | PHE | 174 | −13.123 | 28.067 | 20.811 | 1.00 | 0.00 | −0.650 | 9.00 | −17.40 |
| ATOM | 2797 | HN | PHE | 174 | −13.337 | 27.733 | 21.761 | 1.00 | 0.00 | 0.440 | 0.00 | 0.00 |
| ATOM | 2798 | CA | PHE | 174 | −12.518 | 29.357 | 20.609 | 1.00 | 0.00 | 0.158 | 9.40 | 4.00 |
| ATOM | 2799 | HA | PHE | 174 | −13.239 | 29.962 | 20.060 | 1.00 | 0.00 | 0.053 | 0.00 | 0.00 |
| ATOM | 2800 | C | PHE | 174 | −11.264 | 29.130 | 19.822 | 1.00 | 0.00 | 0.396 | 9.82 | 4.00 |
| ATOM | 2801 | O | PHE | 174 | −10.409 | 28.321 | 20.179 | 1.00 | 0.00 | −0.396 | 8.17 | −17.40 |
| ATOM | 2802 | CB | PHE | 174 | −12.147 | 30.086 | 21.912 | 1.00 | 0.00 | −0.106 | 12.77 | 4.00 |
| ATOM | 2803 | HB1 | PHE | 174 | −11.547 | 30.955 | 21.641 | 1.00 | 0.00 | 0.053 | 0.00 | 0.00 |
| ATOM | 2804 | HB2 | PHE | 174 | −11.579 | 29.387 | 22.527 | 1.00 | 0.00 | 0.053 | 0.00 | 0.00 |
| ATOM | 2805 | CG | PHE | 174 | −13.418 | 30.488 | 22.577 | 1.00 | 0.00 | 0.000 | 7.26 | 0.60 |
| ATOM | 2806 | CD1 | PHE | 174 | −14.124 | 29.587 | 23.339 | 1.00 | 0.00 | −0.127 | 10.80 | 0.60 |
| ATOM | 2807 | HD1 | PHE | 174 | −13.750 | 28.569 | 23.456 | 1.00 | 0.00 | 0.127 | 0.00 | 0.00 |
| ATOM | 2808 | CD2 | PHE | 174 | −13.903 | 31.770 | 22.442 | 1.00 | 0.00 | −0.127 | 10.80 | 0.60 |
| ATOM | 2809 | HD2 | PHE | 174 | −13.352 | 32.494 | 21.842 | 1.00 | 0.00 | 0.127 | 0.00 | 0.00 |
| ATOM | 2810 | CE1 | PHE | 174 | −15.297 | 29.956 | 23.955 | 1.00 | 0.00 | −0.127 | 10.80 | 0.60 |
| ATOM | 2811 | HE1 | PHE | 174 | −15.848 | 29.232 | 24.555 | 1.00 | 0.00 | 0.127 | 0.00 | 0.00 |
| ATOM | 2812 | CE2 | PHE | 174 | −15.075 | 32.145 | 23.056 | 1.00 | 0.00 | −0.127 | 10.80 | 0.60 |
| ATOM | 2813 | HE2 | PHE | 174 | −15.449 | 33.162 | 22.941 | 1.00 | 0.00 | 0.127 | 0.00 | 0.00 |
| ATOM | 2814 | CZ | PHE | 174 | −15.774 | 31.237 | 23.814 | 1.00 | 0.00 | −0.127 | 10.80 | 0.60 |
| ATOM | 2815 | HZ | PHE | 174 | −16.703 | 31.531 | 24.301 | 1.00 | 0.00 | 0.127 | 0.00 | 0.00 |
| ATOM | 2816 | N | PRO | 175 | −11.189 | 29.818 | 18.719 | 1.00 | 0.00 | −0.422 | 9.00 | −17.40 |
| ATOM | 2817 | CA | PRO | 175 | −10.109 | 29.642 | 17.784 | 1.00 | 0.00 | 0.158 | 9.40 | 4.00 |
| ATOM | 2818 | HA | PRO | 175 | −9.859 | 28.581 | 17.750 | 1.00 | 0.00 | 0.053 | 0.00 | 0.00 |
| ATOM | 2819 | CD | PRO | 175 | −12.386 | 30.341 | 18.081 | 1.00 | 0.00 | 0.105 | 12.77 | 4.00 |
| ATOM | 2820 | HD1 | PRO | 175 | −12.761 | 31.093 | 18.775 | 1.00 | 0.00 | 0.053 | 0.00 | 0.00 |
| ATOM | 2821 | HD2 | PRO | 175 | −13.040 | 29.475 | 17.972 | 1.00 | 0.00 | 0.053 | 0.00 | 0.00 |
| ATOM | 2822 | C | PRO | 175 | −8.858 | 30.384 | 18.116 | 1.00 | 0.00 | 0.396 | 9.82 | 4.00 |
| ATOM | 2823 | O | PRO | 175 | −8.906 | 31.353 | 18.872 | 1.00 | 0.00 | −0.396 | 8.17 | −17.40 |
| ATOM | 2824 | CB | PRO | 175 | −10.664 | 30.055 | 16.419 | 1.00 | 0.00 | −0.106 | 12.77 | 4.00 |
| ATOM | 2825 | HB1 | PRO | 175 | −10.904 | 29.113 | 15.924 | 1.00 | 0.00 | 0.053 | 0.00 | 0.00 |
| ATOM | 2826 | HB2 | PRO | 175 | −9.850 | 30.606 | 15.947 | 1.00 | 0.00 | 0.053 | 0.00 | 0.00 |
| ATOM | 2827 | CG | PRO | 175 | −11.893 | 30.918 | 16.747 | 1.00 | 0.00 | −0.106 | 12.77 | 4.00 |
| ATOM | 2828 | HG1 | PRO | 175 | −12.649 | 30.834 | 15.966 | 1.00 | 0.00 | 0.053 | 0.00 | 0.00 |
| ATOM | 2829 | HG2 | PRO | 175 | −11.619 | 31.969 | 16.837 | 1.00 | 0.00 | 0.053 | 0.00 | 0.00 |
| ATOM | 2830 | N | ILE | 176 | −7.723 | 29.924 | 17.553 | 1.00 | 0.00 | −0.650 | 9.00 | −17.40 |
| ATOM | 2831 | HN | ILE | 176 | −7.758 | 29.055 | 17.000 | 1.00 | 0.00 | 0.440 | 0.00 | 0.00 |
| ATOM | 2832 | CA | ILE | 176 | −6.472 | 30.601 | 17.697 | 1.00 | 0.00 | 0.158 | 9.40 | 4.00 |
| ATOM | 2833 | HA | ILE | 176 | −6.611 | 31.589 | 18.135 | 1.00 | 0.00 | 0.053 | 0.00 | 0.00 |
| ATOM | 2834 | C | ILE | 176 | −5.953 | 30.685 | 16.291 | 1.00 | 0.00 | 0.396 | 9.82 | 4.00 |
| ATOM | 2835 | O | ILE | 176 | −6.123 | 29.750 | 15.510 | 1.00 | 0.00 | −0.396 | 8.17 | −17.40 |
| ATOM | 2836 | CB | ILE | 176 | −5.490 | 29.845 | 18.552 | 1.00 | 0.00 | −0.053 | 9.40 | 4.00 |
| ATOM | 2837 | HB | ILE | 176 | −6.012 | 29.473 | 19.433 | 1.00 | 0.00 | 0.053 | 0.00 | 0.00 |
| ATOM | 2838 | CG1 | ILE | 176 | −4.423 | 30.787 | 19.136 | 1.00 | 0.00 | −0.106 | 12.77 | 4.00 |
| ATOM | 2839 | HG1 | ILE | 176 | −3.737 | 30.192 | 19.739 | 1.00 | 0.00 | 0.053 | 0.00 | 0.00 |
| ATOM | 2840 | HG1 | ILE | 176 | −4.927 | 31.534 | 19.748 | 1.00 | 0.00 | 0.053 | 0.00 | 0.00 |
| ATOM | 2841 | CG2 | ILE | 176 | −4.926 | 28.675 | 17.730 | 1.00 | 0.00 | −0.159 | 16.15 | 4.00 |
| ATOM | 2842 | HG2 | ILE | 176 | −4.212 | 28.116 | 18.335 | 1.00 | 0.00 | 0.053 | 0.00 | 0.00 |
| ATOM | 2843 | HG2 | ILE | 176 | −5.740 | 28.016 | 17.428 | 1.00 | 0.00 | 0.053 | 0.00 | 0.00 |
| ATOM | 2844 | HG2 | ILE | 176 | −4.425 | 29.061 | 16.842 | 1.00 | 0.00 | 0.053 | 0.00 | 0.00 |
| ATOM | 2845 | CD1 | ILE | 176 | −3.593 | 31.530 | 18.098 | 1.00 | 0.00 | −0.159 | 16.15 | 4.00 |
| ATOM | 2846 | HD1 | ILE | 176 | −2.868 | 32.169 | 18.601 | 1.00 | 0.00 | 0.053 | 0.00 | 0.00 |
| ATOM | 2847 | HD1 | ILE | 176 | −3.067 | 30.810 | 17.469 | 1.00 | 0.00 | 0.053 | 0.00 | 0.00 |
| ATOM | 2848 | HD1 | ILE | 176 | −4.248 | 32.142 | 17.478 | 1.00 | 0.00 | 0.053 | 0.00 | 0.00 |
| ATOM | 2849 | N | LYS | 177S | −5.347 | 31.823 | 15.908 | 1.00 | 0.00 | −0.650 | 9.00 | −17.40 |
| ATOM | 2850 | HN | LYS | 177S | −5.200 | 32.580 | 16.590 | 1.00 | 0.00 | 0.440 | 0.00 | 0.00 |
| ATOM | 2851 | CA | LYS | 177S | −4.903 | 31.988 | 14.553 | 1.00 | 0.00 | 0.158 | 9.40 | 4.00 |
| ATOM | 2852 | HA | LYS | 177S | −5.712 | 31.793 | 13.848 | 1.00 | 0.00 | 0.053 | 0.00 | 0.00 |
| ATOM | 2853 | C | LYS | 177S | −3.783 | 31.047 | 14.235 | 1.00 | 0.00 | 0.396 | 9.82 | 4.00 |
| ATOM | 2854 | O | LYS | 177S | −3.774 | 30.419 | 13.177 | 1.00 | 0.00 | −0.396 | 8.17 | −17.40 |
| ATOM | 2855 | CB | LYS | 177S | −4.406 | 33.413 | 14.242 | 1.00 | 0.00 | −0.106 | 12.77 | 4.00 |
| ATOM | 2856 | HB1 | LYS | 177S | −5.210 | 34.110 | 14.473 | 1.00 | 0.00 | 0.053 | 0.00 | 0.00 |
| ATOM | 2857 | HB2 | LYS | 177S | −4.147 | 33.458 | 13.184 | 1.00 | 0.00 | 0.053 | 0.00 | 0.00 |
| ATOM | 2858 | CG | LYS | 177S | −3.173 | 33.857 | 15.034 | 1.00 | 0.00 | −0.106 | 12.77 | 4.00 |
| ATOM | 2859 | HG1 | LYS | 177S | −2.845 | 34.821 | 14.645 | 1.00 | 0.00 | 0.053 | 0.00 | 0.00 |
| ATOM | 2860 | HG2 | LYS | 177S | −2.393 | 33.106 | 14.903 | 1.00 | 0.00 | 0.053 | 0.00 | 0.00 |
| ATOM | 2861 | CD | LYS | 177S | −3.416 | 34.019 | 16.535 | 1.00 | 0.00 | −0.106 | 12.77 | 4.00 |
| ATOM | 2862 | HD1 | LYS | 177S | −3.754 | 33.104 | 17.021 | 1.00 | 0.00 | 0.053 | 0.00 | 0.00 |
| ATOM | 2863 | HD2 | LYS | 177S | −4.172 | 34.766 | 16.774 | 1.00 | 0.00 | 0.053 | 0.00 | 0.00 |
| ATOM | 2864 | CE | LYS | 177S | −2.169 | 34.448 | 17.312 | 1.00 | 0.00 | 0.099 | 12.77 | 4.00 |
| ATOM | 2865 | HE1 | LYS | 177S | −1.789 | 35.392 | 16.921 | 1.00 | 0.00 | 0.053 | 0.00 | 0.00 |
| ATOM | 2866 | HE2 | LYS | 177S | −1.390 | 33.691 | 17.219 | 1.00 | 0.00 | 0.053 | 0.00 | 0.00 |
| ATOM | 2867 | NZ | LYS | 177S | −2.496 | 34.622 | 18.745 | 1.00 | 0.00 | −0.045 | 13.25 | −39.20 |
| ATOM | 2868 | HZ1 | LYS | 177S | −1.650 | 34.910 | 19.258 | 1.00 | 0.00 | 0.280 | 0.00 | 0.00 |
| ATOM | 2869 | HZ2 | LYS | 177S | −3.222 | 35.345 | 18.846 | 1.00 | 0.00 | 0.280 | 0.00 | 0.00 |
| ATOM | 2870 | HZ3 | LYS | 177S | −2.843 | 33.731 | 19.129 | 1.00 | 0.00 | 0.280 | 0.00 | 0.00 |
| ATOM | 2871 | N | THR | 178 | −2.806 | 30.913 | 15.149 | 1.00 | 0.00 | −0.650 | 9.00 | −17.40 |
| ATOM | 2872 | HN | THR | 178 | −2.891 | 31.372 | 16.067 | 1.00 | 0.00 | 0.440 | 0.00 | 0.00 |
| ATOM | 2873 | CA | THR | 178 | −1.645 | 30.130 | 14.843 | 1.00 | 0.00 | 0.158 | 9.40 | 4.00 |
| ATOM | 2874 | HA | THR | 178 | −1.929 | 29.404 | 14.081 | 1.00 | 0.00 | 0.053 | 0.00 | 0.00 |
| ATOM | 2875 | C | THR | 178 | −1.211 | 29.451 | 16.109 | 1.00 | 0.00 | 0.396 | 9.82 | 4.00 |
| ATOM | 2876 | O | THR | 178 | −1.683 | 29.790 | 17.192 | 1.00 | 0.00 | −0.396 | 8.17 | −17.40 |
| ATOM | 2877 | CB | THR | 178 | −0.528 | 31.027 | 14.375 | 1.00 | 0.00 | 0.060 | 9.40 | 4.00 |
| ATOM | 2878 | HB | THR | 178 | −0.263 | 31.754 | 15.142 | 1.00 | 0.00 | 0.053 | 0.00 | 0.00 |
| ATOM | 2879 | OG1 | THR | 178 | −0.948 | 31.751 | 13.228 | 1.00 | 0.00 | −0.537 | 11.04 | −17.40 |
| ATOM | 2880 | HG1 | THR | 178 | −0.554 | 32.702 | 13.258 | 1.00 | 0.00 | 0.424 | 0.00 | 0.00 |
| ATOM | 2881 | CG2 | THR | 178 | 0.729 | 30.213 | 14.044 | 1.00 | 0.00 | −0.159 | 16.15 | 4.00 |
| ATOM | 2882 | HG2 | THR | 178 | 1.519 | 30.884 | 13.708 | 1.00 | 0.00 | 0.053 | 0.00 | 0.00 |
| ATOM | 2883 | HG2 | THR | 178 | 1.060 | 29.677 | 14.933 | 1.00 | 0.00 | 0.053 | 0.00 | 0.00 |
| ATOM | 2884 | HG2 | THR | 178 | 0.501 | 29.497 | 13.253 | 1.00 | 0.00 | 0.053 | 0.00 | 0.00 |
| ATOM | 2885 | N | ASN | 179 | −0.299 | 28.462 | 15.992 | 1.00 | 0.00 | −0.650 | 9.00 | −17.40 |
| ATOM | 2886 | HN | ASN | 179 | 0.019 | 28.200 | 15.047 | 1.00 | 0.00 | 0.440 | 0.00 | 0.00 |
| ATOM | 2887 | CA | ASN | 179 | 0.251 | 27.755 | 17.115 | 1.00 | 0.00 | 0.158 | 9.40 | 4.00 |
| ATOM | 2888 | HA | ASN | 179 | 0.792 | 26.872 | 16.773 | 1.00 | 0.00 | 0.053 | 0.00 | 0.00 |
| ATOM | 2889 | C | ASN | 179 | −0.843 | 27.323 | 18.036 | 1.00 | 0.00 | 0.396 | 9.82 | 4.00 |
| ATOM | 2890 | O | ASN | 179 | −1.029 | 27.886 | 19.112 | 1.00 | 0.00 | −0.396 | 8.17 | −17.40 |
| ATOM | 2891 | CB | ASN | 179 | 1.265 | 28.587 | 17.922 | 1.00 | 0.00 | −0.106 | 12.77 | 4.00 |
| ATOM | 2892 | HB1 | ASN | 179 | 1.523 | 28.031 | 18.823 | 1.00 | 0.00 | 0.053 | 0.00 | 0.00 |
| ATOM | 2893 | HB2 | ASN | 179 | 0.799 | 29.539 | 18.177 | 1.00 | 0.00 | 0.053 | 0.00 | 0.00 |
| ATOM | 2894 | CG | ASN | 179 | 2.499 | 28.808 | 17.056 | 1.00 | 0.00 | 0.396 | 9.82 | 4.00 |
| ATOM | 2895 | OD1 | ASN | 179 | 2.738 | 28.084 | 16.091 | 1.00 | 0.00 | −0.396 | 8.17 | −17.40 |
| ATOM | 2896 | ND2 | ASN | 179 | 3.312 | 29.838 | 17.413 | 1.00 | 0.00 | −0.879 | 13.25 | −17.40 |
| ATOM | 2897 | HD2 | ASN | 179 | 3.076 | 30.421 | 18.228 | 1.00 | 0.00 | 0.440 | 0.00 | 0.00 |
| ATOM | 2898 | HD2 | ASN | 179 | 4.163 | 30.034 | 16.867 | 1.00 | 0.00 | 0.440 | 0.00 | 0.00 |
| ATOM | 2899 | N | LEU | 180 | −1.607 | 26.302 | 17.614 | 1.00 | 0.00 | −0.650 | 9.00 | −17.40 |
| ATOM | 2900 | HN | LEU | 180 | −1.408 | 25.882 | 16.694 | 1.00 | 0.00 | 0.440 | 0.00 | 0.00 |
| ATOM | 2901 | CA | LEU | 180 | −2.687 | 25.775 | 18.394 | 1.00 | 0.00 | 0.158 | 9.40 | 4.00 |
| ATOM | 2902 | HA | LEU | 180 | −3.325 | 26.602 | 18.704 | 1.00 | 0.00 | 0.053 | 0.00 | 0.00 |
| ATOM | 2903 | C | LEU | 180 | −2.112 | 25.077 | 19.594 | 1.00 | 0.00 | 0.396 | 9.82 | 4.00 |
| ATOM | 2904 | O | LEU | 180 | −2.776 | 24.939 | 20.620 | 1.00 | 0.00 | −0.396 | 8.17 | −17.40 |
| ATOM | 2905 | CB | LEU | 180 | −3.587 | 24.807 | 17.613 | 1.00 | 0.00 | −0.106 | 12.77 | 4.00 |
| ATOM | 2906 | HB1 | LEU | 180 | −3.072 | 23.890 | 17.325 | 1.00 | 0.00 | 0.053 | 0.00 | 0.00 |
| ATOM | 2907 | HB2 | LEU | 180 | −3.969 | 25.237 | 16.687 | 1.00 | 0.00 | 0.053 | 0.00 | 0.00 |
| ATOM | 2908 | CG | LEU | 180 | −4.824 | 24.363 | 18.414 | 1.00 | 0.00 | −0.053 | 9.40 | 4.00 |
| ATOM | 2909 | HG | LEU | 180 | −4.537 | 23.819 | 19.314 | 1.00 | 0.00 | 0.053 | 0.00 | 0.00 |
| ATOM | 2910 | CD1 | LEU | 180 | −5.670 | 25.572 | 18.848 | 1.00 | 0.00 | −0.159 | 16.15 | 4.00 |
| ATOM | 2911 | HD1 | LEU | 180 | −6.536 | 25.226 | 19.411 | 1.00 | 0.00 | 0.053 | 0.00 | 0.00 |
| ATOM | 2912 | HD1 | LEU | 180 | −5.068 | 26.230 | 19.474 | 1.00 | 0.00 | 0.053 | 0.00 | 0.00 |
| ATOM | 2913 | HD1 | LEU | 180 | −6.005 | 26.117 | 17.965 | 1.00 | 0.00 | 0.053 | 0.00 | 0.00 |
| ATOM | 2914 | CD2 | LEU | 180 | −5.655 | 23.340 | 17.626 | 1.00 | 0.00 | −0.159 | 16.15 | 4.00 |
| ATOM | 2915 | HD2 | LEU | 180 | −6.522 | 23.044 | 18.215 | 1.00 | 0.00 | 0.053 | 0.00 | 0.00 |
| ATOM | 2916 | HD2 | LEU | 180 | −5.988 | 23.786 | 16.689 | 1.00 | 0.00 | 0.053 | 0.00 | 0.00 |
| ATOM | 2917 | HD2 | LEU | 180 | −5.044 | 22.462 | 17.412 | 1.00 | 0.00 | 0.053 | 0.00 | 0.00 |
| ATOM | 2918 | N | GLU | 181 | −0.839 | 24.642 | 19.490 | 1.00 | 0.00 | −0.650 | 9.00 | −17.40 |
| ATOM | 2919 | HN | GLU | 181 | −0.337 | 24.850 | 18.615 | 1.00 | 0.00 | 0.440 | 0.00 | 0.00 |
| ATOM | 2920 | CA | GLU | 181 | −0.125 | 23.909 | 20.506 | 1.00 | 0.00 | 0.158 | 9.40 | 4.00 |
| ATOM | 2921 | HA | GLU | 181 | −0.633 | 22.961 | 20.682 | 1.00 | 0.00 | 0.053 | 0.00 | 0.00 |
| ATOM | 2922 | C | GLU | 181 | −0.102 | 24.718 | 21.764 | 1.00 | 0.00 | 0.396 | 9.82 | 4.00 |
| ATOM | 2923 | O | GLU | 181 | 0.136 | 24.185 | 22.847 | 1.00 | 0.00 | −0.396 | 8.17 | −17.40 |
| ATOM | 2924 | CB | GLU | 181 | 1.340 | 23.602 | 20.144 | 1.00 | 0.00 | −0.106 | 12.77 | 4.00 |
| ATOM | 2925 | HB1 | GLU | 181 | 1.860 | 24.547 | 19.991 | 1.00 | 0.00 | 0.053 | 0.00 | 0.00 |
| ATOM | 2926 | HB2 | GLU | 181 | 1.349 | 23.007 | 19.230 | 1.00 | 0.00 | 0.053 | 0.00 | 0.00 |
| ATOM | 2927 | CG | GLU | 181 | 2.101 | 22.819 | 21.217 | 1.00 | 0.00 | −0.106 | 12.77 | 4.00 |
| ATOM | 2928 | HG1 | GLU | 181 | 2.187 | 21.777 | 20.906 | 1.00 | 0.00 | 0.053 | 0.00 | 0.00 |
| ATOM | 2929 | HG2 | GLU | 181 | 1.554 | 22.878 | 22.158 | 1.00 | 0.00 | 0.053 | 0.00 | 0.00 |
| ATOM | 2930 | CD | GLU | 181 | 3.486 | 23.422 | 21.387 | 1.00 | 0.00 | 0.399 | 9.82 | 4.00 |
| ATOM | 2931 | OE1 | GLU | 181 | 3.757 | 23.995 | 22.476 | 1.00 | 0.00 | −0.396 | 8.17 | −18.95 |
| ATOM | 2932 | OE2 | GLU | 181 | 4.293 | 23.313 | 20.426 | 1.00 | 0.00 | −0.427 | 8.17 | −18.95 |
| ATOM | 2934 | N | ILE | 182 | −0.367 | 26.033 | 21.654 | 1.00 | 0.00 | −0.650 | 9.00 | −17.40 |
| ATOM | 2935 | HN | ILE | 182 | −0.584 | 26.403 | 20.717 | 1.00 | 0.00 | 0.440 | 0.00 | 0.00 |
| ATOM | 2936 | CA | ILE | 182 | −0.366 | 26.955 | 22.758 | 1.00 | 0.00 | 0.158 | 9.40 | 4.00 |
| ATOM | 2937 | HA | ILE | 182 | 0.651 | 27.033 | 23.140 | 1.00 | 0.00 | 0.053 | 0.00 | 0.00 |
| ATOM | 2938 | C | ILE | 182 | −1.287 | 26.425 | 23.820 | 1.00 | 0.00 | 0.396 | 9.82 | 4.00 |
| ATOM | 2939 | O | ILE | 182 | −1.167 | 26.786 | 24.989 | 1.00 | 0.00 | −0.396 | 8.17 | −17.40 |
| ATOM | 2940 | CB | ILE | 182 | −0.835 | 28.333 | 22.374 | 1.00 | 0.00 | −0.053 | 9.40 | 4.00 |
| ATOM | 2941 | HB | ILE | 182 | −1.794 | 28.243 | 21.864 | 1.00 | 0.00 | 0.053 | 0.00 | 0.00 |
| ATOM | 2942 | CG1 | ILE | 182 | 0.131 | 28.957 | 21.354 | 1.00 | 0.00 | −0.106 | 12.77 | 4.00 |
| ATOM | 2943 | HG1 | ILE | 182 | −0.173 | 29.949 | 21.020 | 1.00 | 0.00 | 0.053 | 0.00 | 0.00 |
| ATOM | 2944 | HG1 | ILE | 182 | 0.238 | 28.367 | 20.443 | 1.00 | 0.00 | 0.053 | 0.00 | 0.00 |
| ATOM | 2945 | CG2 | ILE | 182 | −0.985 | 29.174 | 23.654 | 1.00 | 0.00 | −0.159 | 16.15 | 4.00 |
| ATOM | 2946 | HG2 | ILE | 182 | −1.324 | 30.176 | 23.393 | 1.00 | 0.00 | 0.053 | 0.00 | 0.00 |
| ATOM | 2947 | HG2 | ILE | 182 | −1.714 | 28.704 | 24.314 | 1.00 | 0.00 | 0.053 | 0.00 | 0.00 |
| ATOM | 2948 | HG2 | ILE | 182 | −0.022 | 29.237 | 24.162 | 1.00 | 0.00 | 0.053 | 0.00 | 0.00 |
| ATOM | 2949 | CD1 | ILE | 182 | 1.551 | 29.131 | 21.891 | 1.00 | 0.00 | −0.159 | 16.15 | 4.00 |
| ATOM | 2950 | HD1 | ILE | 182 | 2.179 | 29.576 | 21.119 | 1.00 | 0.00 | 0.053 | 0.00 | 0.00 |
| ATOM | 2951 | HD1 | ILE | 182 | 1.532 | 29.782 | 22.764 | 1.00 | 0.00 | 0.053 | 0.00 | 0.00 |
| ATOM | 2952 | HD1 | ILE | 182 | 1.955 | 28.158 | 22.172 | 1.00 | 0.00 | 0.053 | 0.00 | 0.00 |
| ATOM | 2953 | N | VAL | 183 | −2.205 | 25.511 | 23.445 | 1.00 | 0.00 | −0.650 | 9.00 | −17.40 |
| ATOM | 2954 | HN | VAL | 183 | −2.224 | 25.244 | 22.450 | 1.00 | 0.00 | 0.440 | 0.00 | 0.00 |
| ATOM | 2955 | CA | VAL | 183 | −3.160 | 24.876 | 24.315 | 1.00 | 0.00 | 0.158 | 9.40 | 4.00 |
| ATOM | 2956 | HA | VAL | 183 | −3.882 | 25.625 | 24.637 | 1.00 | 0.00 | 0.053 | 0.00 | 0.00 |
| ATOM | 2957 | C | VAL | 183 | −2.424 | 24.313 | 25.494 | 1.00 | 0.00 | 0.396 | 9.82 | 4.00 |
| ATOM | 2958 | O | VAL | 183 | −3.009 | 24.099 | 26.554 | 1.00 | 0.00 | −0.396 | 8.17 | −17.40 |
| ATOM | 2959 | CB | VAL | 183 | −3.853 | 23.725 | 23.648 | 1.00 | 0.00 | −0.053 | 9.40 | 4.00 |
| ATOM | 2960 | HB | VAL | 183 | −4.439 | 24.116 | 22.816 | 1.00 | 0.00 | 0.053 | 0.00 | 0.00 |
| ATOM | 2961 | CG1 | VAL | 183 | −2.780 | 22.745 | 23.143 | 1.00 | 0.00 | −0.159 | 16.15 | 4.00 |
| ATOM | 2962 | HG1 | VAL | 183 | −3.261 | 21.898 | 22.653 | 1.00 | 0.00 | 0.053 | 0.00 | 0.00 |
| ATOM | 2963 | HG1 | VAL | 183 | −2.129 | 23.253 | 22.431 | 1.00 | 0.00 | 0.053 | 0.00 | 0.00 |
| ATOM | 2964 | HG1 | VAL | 183 | −2.187 | 22.388 | 23.985 | 1.00 | 0.00 | 0.053 | 0.00 | 0.00 |
| ATOM | 2965 | CG2 | VAL | 183 | −4.817 | 23.078 | 24.657 | 1.00 | 0.00 | −0.159 | 16.15 | 4.00 |
| ATOM | 2966 | HG2 | VAL | 183 | −5.328 | 22.238 | 24.185 | 1.00 | 0.00 | 0.053 | 0.00 | 0.00 |
| ATOM | 2967 | HG2 | VAL | 183 | −4.254 | 22.721 | 25.520 | 1.00 | 0.00 | 0.053 | 0.00 | 0.00 |
| ATOM | 2968 | HG2 | VAL | 183 | −5.552 | 23.814 | 24.981 | 1.00 | 0.00 | 0.053 | 0.00 | 0.00 |
| ATOM | 2969 | N | ARG | 184G | −1.112 | 24.071 | 25.337 | 1.00 | 0.00 | −0.650 | 9.00 | −17.40 |
| ATOM | 2970 | HN | ARG | 184G | −0.681 | 24.289 | 24.427 | 1.00 | 0.00 | 0.440 | 0.00 | 0.00 |
| ATOM | 2971 | CA | ARG | 184G | −0.282 | 23.526 | 26.366 | 1.00 | 0.00 | 0.158 | 9.40 | 4.00 |
| ATOM | 2972 | HA | ARG | 184G | −0.587 | 22.492 | 26.527 | 1.00 | 0.00 | 0.053 | 0.00 | 0.00 |
| ATOM | 2973 | C | ARG | 184G | −0.486 | 24.349 | 27.602 | 1.00 | 0.00 | 0.396 | 9.82 | 4.00 |
| ATOM | 2974 | O | ARG | 184G | −0.357 | 23.827 | 28.708 | 1.00 | 0.00 | −0.396 | 8.17 | −17.40 |
| ATOM | 2975 | CB | ARG | 184G | 1.212 | 23.628 | 26.013 | 1.00 | 0.00 | −0.106 | 12.77 | 4.00 |
| ATOM | 2976 | HB1 | ARG | 184G | 1.500 | 24.678 | 26.038 | 1.00 | 0.00 | 0.053 | 0.00 | 0.00 |
| ATOM | 2977 | HB2 | ARG | 184G | 1.358 | 23.215 | 25.015 | 1.00 | 0.00 | 0.053 | 0.00 | 0.00 |
| ATOM | 2978 | CG | ARG | 184G | 2.129 | 22.869 | 26.972 | 1.00 | 0.00 | −0.106 | 12.77 | 4.00 |
| ATOM | 2979 | HG1 | ARG | 184G | 1.815 | 22.946 | 28.013 | 1.00 | 0.00 | 0.053 | 0.00 | 0.00 |
| ATOM | 2980 | HG2 | ARG | 184G | 3.159 | 23.223 | 26.947 | 1.00 | 0.00 | 0.053 | 0.00 | 0.00 |
| ATOM | 2981 | CD | ARG | 184G | 2.200 | 21.372 | 26.673 | 1.00 | 0.00 | 0.374 | 12.77 | 4.00 |
| ATOM | 2982 | HD1 | ARG | 184G | 1.225 | 21.075 | 26.284 | 1.00 | 0.00 | 0.053 | 0.00 | 0.00 |
| ATOM | 2983 | HD2 | ARG | 184G | 2.430 | 20.867 | 27.611 | 1.00 | 0.00 | 0.053 | 0.00 | 0.00 |
| ATOM | 2984 | NE | ARG | 184G | 3.279 | 21.177 | 25.663 | 1.00 | 0.00 | −0.819 | 9.00 | −24.67 |
| ATOM | 2985 | HE | ARG | 184G | 4.226 | 20.913 | 25.970 | 1.00 | 0.00 | 0.407 | 0.00 | 0.00 |
| ATOM | 2986 | CZ | ARG | 184G | 3.019 | 21.346 | 24.333 | 1.00 | 0.00 | 0.796 | 6.95 | 4.00 |
| ATOM | 2987 | NH1 | ARG | 184G | 1.770 | 21.709 | 23.920 | 1.00 | 0.00 | −0.746 | 9.00 | −24.67 |
| ATOM | 2988 | HH1 | ARG | 184G | 1.576 | 21.835 | 22.916 | 1.00 | 0.00 | 0.407 | 0.00 | 0.00 |
| ATOM | 2989 | HH1 | ARG | 184G | 1.021 | 21.855 | 24.611 | 1.00 | 0.00 | 0.407 | 0.00 | 0.00 |
| ATOM | 2990 | NH2 | ARG | 184G | 4.012 | 21.157 | 23.416 | 1.00 | 0.00 | −0.746 | 9.00 | −24.67 |
| ATOM | 2991 | HH2 | ARG | 184G | 3.817 | 21.284 | 22.412 | 1.00 | 0.00 | 0.407 | 0.00 | 0.00 |
| ATOM | 2992 | HH2 | ARG | 184G | 4.956 | 20.887 | 23.727 | 1.00 | 0.00 | 0.407 | 0.00 | 0.00 |
| ATOM | 2993 | N | LYS | 185S | −0.786 | 25.658 | 27.444 | 1.00 | 0.00 | −0.650 | 9.00 | −17.40 |
| ATOM | 2994 | HN | LYS | 185S | −0.867 | 26.039 | 26.490 | 1.00 | 0.00 | 0.440 | 0.00 | 0.00 |
| ATOM | 2995 | CA | LYS | 185S | −0.995 | 26.535 | 28.567 | 1.00 | 0.00 | 0.158 | 9.40 | 4.00 |
| ATOM | 2996 | HA | LYS | 185S | −0.040 | 26.709 | 29.064 | 1.00 | 0.00 | 0.053 | 0.00 | 0.00 |
| ATOM | 2997 | C | LYS | 185S | −1.955 | 25.890 | 29.519 | 1.00 | 0.00 | 0.396 | 9.82 | 4.00 |
| ATOM | 2998 | O | LYS | 185S | −2.830 | 25.122 | 29.122 | 1.00 | 0.00 | −0.396 | 8.17 | −17.40 |
| ATOM | 2999 | CB | LYS | 185S | −1.576 | 27.912 | 28.202 | 1.00 | 0.00 | −0.106 | 12.77 | 4.00 |
| ATOM | 3000 | HB1 | LYS | 185S | −1.038 | 28.407 | 27.393 | 1.00 | 0.00 | 0.053 | 0.00 | 0.00 |
| ATOM | 3001 | HB2 | LYS | 185S | −1.565 | 28.619 | 29.031 | 1.00 | 0.00 | 0.053 | 0.00 | 0.00 |
| ATOM | 3002 | CG | LYS | 185S | −3.033 | 27.853 | 27.741 | 1.00 | 0.00 | −0.106 | 12.77 | 4.00 |
| ATOM | 3003 | HG1 | LYS | 185S | −3.659 | 27.211 | 28.360 | 1.00 | 0.00 | 0.053 | 0.00 | 0.00 |
| ATOM | 3004 | HG2 | LYS | 185S | −3.153 | 27.473 | 26.726 | 1.00 | 0.00 | 0.053 | 0.00 | 0.00 |
| ATOM | 3005 | CD | LYS | 185S | −3.730 | 29.215 | 27.743 | 1.00 | 0.00 | −0.106 | 12.77 | 4.00 |
| ATOM | 3006 | HD1 | LYS | 1855 | −3.260 | 29.947 | 27.085 | 1.00 | 0.00 | 0.053 | 0.00 | 0.00 |
| ATOM | 3007 | HD2 | LYS | 185S | −3.755 | 29.691 | 28.723 | 1.00 | 0.00 | 0.053 | 0.00 | 0.00 |
| ATOM | 3008 | CE | LYS | 185S | −5.191 | 29.157 | 27.292 | 1.00 | 0.00 | 0.099 | 12.77 | 4.00 |
| ATOM | 3009 | HE1 | LYS | 185S | −5.740 | 28.438 | 27.900 | 1.00 | 0.00 | 0.053 | 0.00 | 0.00 |
| ATOM | 3010 | HE2 | LYS | 185S | −5.247 | 28.851 | 26.247 | 1.00 | 0.00 | 0.053 | 0.00 | 0.00 |
| ATOM | 3011 | NZ | LYS | 185S | −5.819 | 30.489 | 27.437 | 1.00 | 0.00 | −0.045 | 13.25 | −39.20 |
| ATOM | 3012 | HZ1 | LYS | 185S | −6.801 | 30.441 | 27.131 | 1.00 | 0.00 | 0.280 | 0.00 | 0.00 |
| ATOM | 3013 | HZ2 | LYS | 185S | −5.780 | 30.781 | 28.423 | 1.00 | 0.00 | 0.280 | 0.00 | 0.00 |
| ATOM | 3014 | HZ3 | LYS | 185S | −5.312 | 31.172 | 26.856 | 1.00 | 0.00 | 0.280 | 0.00 | 0.00 |
| ATOM | 3015 | N | LEU | 186 | −1.789 | 26.195 | 30.820 | 1.00 | 0.00 | −0.650 | 9.00 | −17.40 |
| ATOM | 3016 | HN | LEU | 186 | −1.080 | 26.898 | 31.073 | 1.00 | 0.00 | 0.440 | 0.00 | 0.00 |
| ATOM | 3017 | CA | LEU | 186 | −2.553 | 25.586 | 31.869 | 1.00 | 0.00 | 0.158 | 9.40 | 4.00 |
| ATOM | 3018 | HA | LEU | 186 | −2.500 | 24.498 | 31.827 | 1.00 | 0.00 | 0.053 | 0.00 | 0.00 |
| ATOM | 3019 | C | LEU | 186 | −3.999 | 25.960 | 31.790 | 1.00 | 0.00 | 0.396 | 9.82 | 4.00 |
| ATOM | 3020 | O | LEU | 186 | −4.865 | 25.128 | 32.053 | 1.00 | 0.00 | −0.396 | 8.17 | −17.40 |
| ATOM | 3021 | CB | LEU | 186 | −2.010 | 25.914 | 33.278 | 1.00 | 0.00 | −0.106 | 12.77 | 4.00 |
| ATOM | 3022 | HB1 | LEU | 186 | −0.955 | 25.639 | 33.300 | 1.00 | 0.00 | 0.053 | 0.00 | 0.00 |
| ATOM | 3023 | HB2 | LEU | 186 | −2.582 | 25.331 | 33.999 | 1.00 | 0.00 | 0.053 | 0.00 | 0.00 |
| ATOM | 3024 | CG | LEU | 186 | −2.099 | 27.390 | 33.721 | 1.00 | 0.00 | −0.053 | 9.40 | 4.00 |
| ATOM | 3025 | HG | LEU | 186 | −1.748 | 28.072 | 32.946 | 1.00 | 0.00 | 0.053 | 0.00 | 0.00 |
| ATOM | 3026 | CD1 | LEU | 186 | −3.539 | 27.806 | 34.058 | 1.00 | 0.00 | −0.159 | 16.15 | 4.00 |
| ATOM | 3027 | HD1 | LEU | 186 | −3.553 | 28.851 | 34.364 | 1.00 | 0.00 | 0.053 | 0.00 | 0.00 |
| ATOM | 3028 | HD1 | LEU | 186 | −4.170 | 27.677 | 33.178 | 1.00 | 0.00 | 0.053 | 0.00 | 0.00 |
| ATOM | 3029 | HD1 | LEU | 186 | −3.916 | 27.184 | 34.870 | 1.00 | 0.00 | 0.053 | 0.00 | 0.00 |
| ATOM | 3030 | CD2 | LEU | 186 | −1.117 | 27.682 | 34.867 | 1.00 | 0.00 | −0.159 | 16.15 | 4.00 |
| ATOM | 3031 | HD2 | LEU | 186 | −1.200 | 28.728 | 35.159 | 1.00 | 0.00 | 0.053 | 0.00 | 0.00 |
| ATOM | 3032 | HD2 | LEU | 186 | −1.354 | 27.046 | 35.720 | 1.00 | 0.00 | 0.053 | 0.00 | 0.00 |
| ATOM | 3033 | HD2 | LEU | 186 | −0.099 | 27.478 | 34.534 | 1.00 | 0.00 | 0.053 | 0.00 | 0.00 |
| ATOM | 3034 | N | LYS | 187S | −4.314 | 27.212 | 31.415 | 1.00 | 0.00 | −0.650 | 9.00 | −17.40 |
| ATOM | 3035 | HN | LYS | 187S | −3.583 | 27.870 | 31.109 | 1.00 | 0.00 | 0.440 | 0.00 | 0.00 |
| ATOM | 3036 | CA | LYS | 187S | −5.691 | 27.609 | 31.450 | 1.00 | 0.00 | 0.158 | 9.40 | 4.00 |
| ATOM | 3037 | HA | LYS | 187S | −6.076 | 27.516 | 32.465 | 1.00 | 0.00 | 0.053 | 0.00 | 0.00 |
| ATOM | 3038 | C | LYS | 187S | −6.494 | 26.734 | 30.539 | 1.00 | 0.00 | 0.396 | 9.82 | 4.00 |
| ATOM | 3039 | O | LYS | 187S | −7.522 | 26.189 | 30.940 | 1.00 | 0.00 | −0.396 | 8.17 | −17.40 |
| ATOM | 3040 | CB | LYS | 187S | −5.892 | 29.077 | 31.037 | 1.00 | 0.00 | −0.106 | 12.77 | 4.00 |
| ATOM | 3041 | HB1 | LYS | 187S | −6.942 | 29.329 | 30.893 | 1.00 | 0.00 | 0.053 | 0.00 | 0.00 |
| ATOM | 3042 | HB2 | LYS | 187S | −5.388 | 29.319 | 30.101 | 1.00 | 0.00 | 0.053 | 0.00 | 0.00 |
| ATOM | 3043 | CG | LYS | 187S | −5.353 | 30.059 | 32.082 | 1.00 | 0.00 | −0.106 | 12.77 | 4.00 |
| ATOM | 3044 | HG1 | LYS | 187S | −4.362 | 29.721 | 32.388 | 1.00 | 0.00 | 0.053 | 0.00 | 0.00 |
| ATOM | 3045 | HG2 | LYS | 187S | −6.037 | 30.064 | 32.930 | 1.00 | 0.00 | 0.053 | 0.00 | 0.00 |
| ATOM | 3046 | CD | LYS | 187S | −5.218 | 31.501 | 31.587 | 1.00 | 0.00 | −0.106 | 12.77 | 4.00 |
| ATOM | 3047 | HD1 | LYS | 187S | −6.156 | 31.922 | 31.225 | 1.00 | 0.00 | 0.053 | 0.00 | 0.00 |
| ATOM | 3048 | HD2 | LYS | 187S | −4.514 | 31.609 | 30.761 | 1.00 | 0.00 | 0.053 | 0.00 | 0.00 |
| ATOM | 3049 | CE | LYS | 187S | −4.728 | 32.468 | 32.667 | 1.00 | 0.00 | 0.099 | 12.77 | 4.00 |
| ATOM | 3050 | HE1 | LYS | 187S | −5.399 | 32.446 | 33.525 | 1.00 | 0.00 | 0.053 | 0.00 | 0.00 |
| ATOM | 3051 | HE2 | LYS | 187S | −4.694 | 33.484 | 32.275 | 1.00 | 0.00 | 0.053 | 0.00 | 0.00 |
| ATOM | 3052 | NZ | LYS | 187S | −3.369 | 32.088 | 33.117 | 1.00 | 0.00 | −0.045 | 13.25 | −39.20 |
| ATOM | 3053 | HZ1 | LYS | 187S | −3.050 | 32.745 | 33.843 | 1.00 | 0.00 | 0.280 | 0.00 | 0.00 |
| ATOM | 3054 | HZ2 | LYS | 187S | −3.389 | 31.133 | 33.504 | 1.00 | 0.00 | 0.280 | 0.00 | 0.00 |
| ATOM | 3055 | HZ3 | LYS | 187S | −2.720 | 32.120 | 32.317 | 1.00 | 0.00 | 0.280 | 0.00 | 0.00 |
| ATOM | 3056 | N | LEU | 188 | −6.053 | 26.584 | 29.279 | 1.00 | 0.00 | −0.650 | 9.00 | −17.40 |
| ATOM | 3057 | HN | LEU | 188 | −5.183 | 27.051 | 28.984 | 1.00 | 0.00 | 0.440 | 0.00 | 0.00 |
| ATOM | 3058 | CA | LEU | 188 | −6.779 | 25.780 | 28.340 | 1.00 | 0.00 | 0.158 | 9.40 | 4.00 |
| ATOM | 3059 | HA | LEU | 188 | −7.840 | 26.024 | 28.378 | 1.00 | 0.00 | 0.053 | 0.00 | 0.00 |
| ATOM | 3060 | C | LEU | 188 | −6.606 | 24.325 | 28.663 | 1.00 | 0.00 | 0.396 | 9.82 | 4.00 |
| ATOM | 3061 | O | LEU | 188 | −7.554 | 23.542 | 28.610 | 1.00 | 0.00 | −0.396 | 8.17 | −17.40 |
| ATOM | 3062 | CB | LEU | 188 | −6.332 | 26.037 | 26.891 | 1.00 | 0.00 | −0.106 | 12.77 | 4.00 |
| ATOM | 3063 | HB1 | LEU | 188 | −5.273 | 25.790 | 26.811 | 1.00 | 0.00 | 0.053 | 0.00 | 0.00 |
| ATOM | 3064 | HB2 | LEU | 188 | −6.499 | 27.089 | 26.663 | 1.00 | 0.00 | 0.053 | 0.00 | 0.00 |
| ATOM | 3065 | CG | LEU | 188 | −7.085 | 25.206 | 25.837 | 1.00 | 0.00 | −0.053 | 9.40 | 4.00 |
| ATOM | 3066 | HG | LEU | 188 | −6.924 | 24.139 | 25.991 | 1.00 | 0.00 | 0.053 | 0.00 | 0.00 |
| ATOM | 3067 | CD1 | LEU | 188 | −8.597 | 25.468 | 25.904 | 1.00 | 0.00 | −0.159 | 16.15 | 4.00 |
| ATOM | 3068 | HD1 | LEU | 188 | −9.104 | 24.867 | 25.148 | 1.00 | 0.00 | 0.053 | 0.00 | 0.00 |
| ATOM | 3069 | HD1 | LEU | 188 | −8.969 | 25.197 | 26.892 | 1.00 | 0.00 | 0.053 | 0.00 | 0.00 |
| ATOM | 3070 | HD1 | LEU | 188 | −8.791 | 26.524 | 25.719 | 1.00 | 0.00 | 0.053 | 0.00 | 0.00 |
| ATOM | 3071 | CD2 | LEU | 188 | −6.525 | 25.466 | 24.429 | 1.00 | 0.00 | −0.159 | 16.15 | 4.00 |
| ATOM | 3072 | HD2 | LEU | 188 | −7.074 | 24.866 | 23.702 | 1.00 | 0.00 | 0.053 | 0.00 | 0.00 |
| ATOM | 3073 | HD2 | LEU | 188 | −6.633 | 26.522 | 24.183 | 1.00 | 0.00 | 0.053 | 0.00 | 0.00 |
| ATOM | 3074 | HD2 | LEU | 188 | −5.470 | 25.193 | 24.400 | 1.00 | 0.00 | 0.053 | 0.00 | 0.00 |
| ATOM | 3075 | N | LEU | 189 | −5.372 | 23.937 | 29.035 | 1.00 | 0.00 | −0.650 | 9.00 | −17.40 |
| ATOM | 3076 | HN | LEU | 189 | −4.652 | 24.660 | 29.175 | 1.00 | 0.00 | 0.440 | 0.00 | 0.00 |
| ATOM | 3077 | CA | LEU | 189 | −5.011 | 22.564 | 29.246 | 1.00 | 0.00 | 0.158 | 9.40 | 4.00 |
| ATOM | 3078 | HA | LEU | 189 | −5.221 | 21.999 | 28.337 | 1.00 | 0.00 | 0.053 | 0.00 | 0.00 |
| ATOM | 3079 | C | LEU | 189 | −5.816 | 22.019 | 30.386 | 1.00 | 0.00 | 0.396 | 9.82 | 4.00 |
| ATOM | 3080 | O | LEU | 189 | −6.394 | 20.937 | 30.294 | 1.00 | 0.00 | −0.396 | 8.17 | −17.40 |
| ATOM | 3081 | CB | LEU | 189 | −3.507 | 22.448 | 29.568 | 1.00 | 0.00 | −0.106 | 12.77 | 4.00 |
| ATOM | 3082 | HB1 | LEU | 189 | −3.361 | 22.734 | 30.609 | 1.00 | 0.00 | 0.053 | 0.00 | 0.00 |
| ATOM | 3083 | HB2 | LEU | 189 | −2.966 | 23.120 | 28.901 | 1.00 | 0.00 | 0.053 | 0.00 | 0.00 |
| ATOM | 3084 | CG | LEU | 189 | −2.902 | 21.045 | 29.391 | 1.00 | 0.00 | −0.053 | 9.40 | 4.00 |
| ATOM | 3085 | HG | LEU | 189 | −3.005 | 20.698 | 28.362 | 1.00 | 0.00 | 0.053 | 0.00 | 0.00 |
| ATOM | 3086 | CD1 | LEU | 189 | −1.406 | 21.038 | 29.739 | 1.00 | 0.00 | −0.159 | 16.15 | 4.00 |
| ATOM | 3087 | HD1 | LEU | 189 | −1.005 | 20.033 | 29.604 | 1.00 | 0.00 | 0.053 | 0.00 | 0.00 |
| ATOM | 3088 | HD1 | LEU | 189 | −0.876 | 21.730 | 29.083 | 1.00 | 0.00 | 0.053 | 0.00 | 0.00 |
| ATOM | 3089 | HD1 | LEU | 189 | −1.272 | 21.346 | 30.776 | 1.00 | 0.00 | 0.053 | 0.00 | 0.00 |
| ATOM | 3090 | CD2 | LEU | 189 | −3.699 | 19.980 | 30.143 | 1.00 | 0.00 | −0.159 | 16.15 | 4.00 |
| ATOM | 3091 | HD2 | LEU | 189 | −3.236 | 19.004 | 29.990 | 1.00 | 0.00 | 0.053 | 0.00 | 0.00 |
| ATOM | 3092 | HD2 | LEU | 189 | −3.707 | 20.215 | 31.207 | 1.00 | 0.00 | 0.053 | 0.00 | 0.00 |
| ATOM | 3093 | HD2 | LEU | 189 | −4.722 | 19.959 | 29.768 | 1.00 | 0.00 | 0.053 | 0.00 | 0.00 |
| ATOM | 3094 | N | MET | 190 | −5.890 | 22.779 | 31.493 | 1.00 | 0.00 | −0.650 | 9.00 | −17.40 |
| ATOM | 3095 | HN | MET | 190 | −5.380 | 23.673 | 31.518 | 1.00 | 0.00 | 0.440 | 0.00 | 0.00 |
| ATOM | 3096 | CA | MET | 190 | −6.655 | 22.385 | 32.638 | 1.00 | 0.00 | 0.158 | 9.40 | 4.00 |
| ATOM | 3097 | HA | MET | 190 | −6.591 | 23.133 | 33.427 | 1.00 | 0.00 | 0.053 | 0.00 | 0.00 |
| ATOM | 3098 | C | MET | 190 | −6.159 | 21.086 | 33.195 | 1.00 | 0.00 | 0.396 | 9.82 | 4.00 |
| ATOM | 3099 | O | MET | 190 | −6.923 | 20.338 | 33.801 | 1.00 | 0.00 | −0.396 | 8.17 | −17.40 |
| ATOM | 3100 | CB | MET | 190 | −8.158 | 22.251 | 32.348 | 1.00 | 0.00 | −0.106 | 12.77 | 4.00 |
| ATOM | 3101 | HB1 | MET | 190 | −8.721 | 21.810 | 33.170 | 1.00 | 0.00 | 0.053 | 0.00 | 0.00 |
| ATOM | 3102 | HB2 | MET | 190 | −8.379 | 21.626 | 31.482 | 1.00 | 0.00 | 0.053 | 0.00 | 0.00 |
| ATOM | 3103 | CG | MET | 190 | −8.845 | 23.588 | 32.063 | 1.00 | 0.00 | −0.041 | 12.77 | 4.00 |
| ATOM | 3104 | HG1 | MET | 190 | −9.861 | 23.383 | 31.727 | 1.00 | 0.00 | 0.053 | 0.00 | 0.00 |
| ATOM | 3105 | HG2 | MET | 190 | −8.278 | 24.103 | 31.286 | 1.00 | 0.00 | 0.053 | 0.00 | 0.00 |
| ATOM | 3106 | SD | MET | 190 | −8.955 | 24.706 | 33.494 | 1.00 | 0.00 | −0.130 | 16.39 | −6.40 |
| ATOM | 3107 | CE | MET | 190 | −7.191 | 25.142 | 33.533 | 1.00 | 0.00 | −0.094 | 16.15 | 4.00 |
| ATOM | 3108 | HE1 | MET | 190 | −7.006 | 25.838 | 34.351 | 1.00 | 0.00 | 0.053 | 0.00 | 0.00 |
| ATOM | 3109 | HE2 | MET | 190 | −6.596 | 24.240 | 33.682 | 1.00 | 0.00 | 0.053 | 0.00 | 0.00 |
| ATOM | 3110 | HE3 | MET | 190 | −6.910 | 25.609 | 32.588 | 1.00 | 0.00 | 0.053 | 0.00 | 0.00 |
| ATOM | 3111 | N | GLY | 191 | −4.859 | 20.785 | 33.026 | 1.00 | 0.00 | −0.650 | 9.00 | −17.40 |
| ATOM | 3112 | HN | GLY | 191 | −4.262 | 21.391 | 32.446 | 1.00 | 0.00 | 0.440 | 0.00 | 0.00 |
| ATOM | 3113 | CA | GLY | 191 | −4.301 | 19.618 | 33.653 | 1.00 | 0.00 | 0.105 | 9.40 | 4.00 |
| ATOM | 3114 | HA1 | GLY | 191 | −4.421 | 19.641 | 34.736 | 1.00 | 0.00 | 0.053 | 0.00 | 0.00 |
| ATOM | 3115 | HA2 | GLY | 191 | −3.233 | 19.521 | 33.454 | 1.00 | 0.00 | 0.053 | 0.00 | 0.00 |
| ATOM | 3116 | C | GLY | 191 | −4.953 | 18.355 | 33.167 | 1.00 | 0.00 | 0.396 | 9.82 | 4.00 |
| ATOM | 3117 | O | GLY | 191 | −5.475 | 17.580 | 33.966 | 1.00 | 0.00 | −0.396 | 8.17 | −17.40 |
| ATOM | 3118 | N | GLU | 192 | −4.943 | 18.113 | 31.842 | 1.00 | 0.00 | −0.650 | 9.00 | −17.40 |
| ATOM | 3119 | HN | GLU | 192 | −4.528 | 18.813 | 31.211 | 1.00 | 0.00 | 0.440 | 0.00 | 0.00 |
| ATOM | 3120 | CA | GLU | 192 | −5.492 | 16.910 | 31.279 | 1.00 | 0.00 | 0.158 | 9.40 | 4.00 |
| ATOM | 3121 | HA | GLU | 192 | −6.420 | 16.713 | 31.814 | 1.00 | 0.00 | 0.053 | 0.00 | 0.00 |
| ATOM | 3122 | C | GLU | 192 | −4.466 | 15.828 | 31.499 | 1.00 | 0.00 | 0.396 | 9.82 | 4.00 |
| ATOM | 3123 | O | GLU | 192 | −3.362 | 16.141 | 31.940 | 1.00 | 0.00 | −0.396 | 8.17 | −17.40 |
| ATOM | 3124 | CB | GLU | 192 | −5.751 | 17.029 | 29.769 | 1.00 | 0.00 | −0.106 | 12.77 | 4.00 |
| ATOM | 3125 | HB1 | GLU | 192 | −6.245 | 16.148 | 29.358 | 1.00 | 0.00 | 0.053 | 0.00 | 0.00 |
| ATOM | 3126 | HB2 | GLU | 192 | −4.832 | 17.156 | 29.195 | 1.00 | 0.00 | 0.053 | 0.00 | 0.00 |
| ATOM | 3127 | CG | GLU | 192 | −6.643 | 18.223 | 29.418 | 1.00 | 0.00 | −0.106 | 12.77 | 4.00 |
| ATOM | 3128 | HG1 | GLU | 192 | −6.994 | 18.106 | 28.392 | 1.00 | 0.00 | 0.053 | 0.00 | 0.00 |
| ATOM | 3129 | HG2 | GLU | 192 | −6.056 | 19.136 | 29.514 | 1.00 | 0.00 | 0.053 | 0.00 | 0.00 |
| ATOM | 3130 | CD | GLU | 192 | −7.822 | 18.249 | 30.380 | 1.00 | 0.00 | 0.399 | 9.82 | 4.00 |
| ATOM | 3131 | OE1 | GLU | 192 | −8.197 | 17.160 | 30.892 | 1.00 | 0.00 | −0.396 | 8.17 | −18.95 |
| ATOM | 3132 | OE2 | GLU | 192 | −8.373 | 19.360 | 30.607 | 1.00 | 0.00 | −0.427 | 8.17 | −18.95 |
| ATOM | 3134 | N | ASN | 193 | −4.792 | 14.537 | 31.204 | 1.00 | 0.00 | −0.650 | 9.00 | −17.40 |
| ATOM | 3135 | HN | ASN | 193 | −5.701 | 14.326 | 30.769 | 1.00 | 0.00 | 0.440 | 0.00 | 0.00 |
| ATOM | 3136 | CA | ASN | 193 | −3.866 | 13.457 | 31.498 | 1.00 | 0.00 | 0.158 | 9.40 | 4.00 |
| ATOM | 3137 | HA | ASN | 193 | −2.921 | 13.841 | 31.882 | 1.00 | 0.00 | 0.053 | 0.00 | 0.00 |
| ATOM | 3138 | C | ASN | 193 | −3.561 | 12.644 | 30.258 | 1.00 | 0.00 | 0.396 | 9.82 | 4.00 |
| ATOM | 3139 | O | ASN | 193 | −4.028 | 12.970 | 29.168 | 1.00 | 0.00 | −0.396 | 8.17 | −17.40 |
| ATOM | 3140 | CB | ASN | 193 | −4.400 | 12.403 | 32.488 | 1.00 | 0.00 | −0.106 | 12.77 | 4.00 |
| ATOM | 3141 | HB1 | ASN | 193 | −4.976 | 12.893 | 33.272 | 1.00 | 0.00 | 0.053 | 0.00 | 0.00 |
| ATOM | 3142 | HB2 | ASN | 193 | −3.565 | 11.865 | 32.939 | 1.00 | 0.00 | 0.053 | 0.00 | 0.00 |
| ATOM | 3143 | CG | ASN | 193 | −5.300 | 11.407 | 31.748 | 1.00 | 0.00 | 0.396 | 9.82 | 4.00 |
| ATOM | 3144 | OD1 | ASN | 193 | −6.056 | 11.716 | 30.828 | 1.00 | 0.00 | −0.396 | 8.17 | −17.40 |
| ATOM | 3145 | ND2 | ASN | 193 | −5.203 | 10.118 | 32.170 | 1.00 | 0.00 | −0.879 | 13.25 | −17.40 |
| ATOM | 3146 | HD2 | ASN | 193 | −4.565 | 9.875 | 32.941 | 1.00 | 0.00 | 0.440 | 0.00 | 0.00 |
| ATOM | 3147 | HD2 | ASN | 193 | −5.767 | 9.384 | 31.718 | 1.00 | 0.00 | 0.440 | 0.00 | 0.00 |
| ATOM | 3148 | N | ASN | 194 | −2.797 | 11.524 | 30.434 | 1.00 | 0.00 | −0.650 | 9.00 | −17.40 |
| ATOM | 3149 | HN | ASN | 194 | −2.515 | 11.292 | 31.397 | 1.00 | 0.00 | 0.440 | 0.00 | 0.00 |
| ATOM | 3150 | CA | ASN | 194 | −2.348 | 10.633 | 29.379 | 1.00 | 0.00 | 0.158 | 9.40 | 4.00 |
| ATOM | 3151 | HA | ASN | 194 | −3.047 | 10.674 | 28.543 | 1.00 | 0.00 | 0.053 | 0.00 | 0.00 |
| ATOM | 3152 | C | ASN | 194 | −2.280 | 9.221 | 29.905 | 1.00 | 0.00 | 0.396 | 9.82 | 4.00 |
| ATOM | 3153 | O | ASN | 194 | −2.837 | 8.905 | 30.955 | 1.00 | 0.00 | −0.396 | 8.17 | −17.40 |
| ATOM | 3154 | CB | ASN | 194 | −0.909 | 10.925 | 28.918 | 1.00 | 0.00 | −0.106 | 12.77 | 4.00 |
| ATOM | 3155 | HB1 | ASN | 194 | −0.533 | 10.114 | 28.293 | 1.00 | 0.00 | 0.053 | 0.00 | 0.00 |
| ATOM | 3156 | HB2 | ASN | 194 | −0.244 | 11.031 | 29.775 | 1.00 | 0.00 | 0.053 | 0.00 | 0.00 |
| ATOM | 3157 | CG | ASN | 194 | −0.855 | 12.213 | 28.111 | 1.00 | 0.00 | 0.396 | 9.82 | 4.00 |
| ATOM | 3158 | OD1 | ASN | 194 | −1.508 | 12.336 | 27.076 | 1.00 | 0.00 | −0.396 | 8.17 | −17.40 |
| ATOM | 3159 | ND2 | ASN | 194 | −0.045 | 13.196 | 28.587 | 1.00 | 0.00 | −0.879 | 13.25 | −17.40 |
| ATOM | 3160 | HD2 | ASN | 194 | 0.484 | 13.050 | 29.458 | 1.00 | 0.00 | 0.440 | 0.00 | 0.00 |
| ATOM | 3161 | HD2 | ASN | 194 | 0.039 | 14.086 | 28.075 | 1.00 | 0.00 | 0.440 | 0.00 | 0.00 |
| ATOM | 3162 | N | LEU | 195 | −1.591 | 8.323 | 29.150 | 1.00 | 0.00 | −0.650 | 9.00 | −17.40 |
| ATOM | 3163 | HN | LEU | 195 | −1.184 | 8.634 | 28.256 | 1.00 | 0.00 | 0.440 | 0.00 | 0.00 |
| ATOM | 3164 | CA | LEU | 195 | −1.409 | 6.951 | 29.555 | 1.00 | 0.00 | 0.158 | 9.40 | 4.00 |
| ATOM | 3165 | HA | LEU | 195 | −1.786 | 6.825 | 30.569 | 1.00 | 0.00 | 0.053 | 0.00 | 0.00 |
| ATOM | 3166 | C | LEU | 195 | 0.053 | 6.615 | 29.511 | 1.00 | 0.00 | 0.396 | 9.82 | 4.00 |
| ATOM | 3167 | O | LEU | 195 | 0.881 | 7.440 | 29.132 | 1.00 | 0.00 | −0.396 | 8.17 | −17.40 |
| ATOM | 3168 | CB | LEU | 195 | −2.165 | 5.919 | 28.700 | 1.00 | 0.00 | −0.106 | 12.77 | 4.00 |
| ATOM | 3169 | HB1 | LEU | 195 | −1.915 | 4.889 | 28.956 | 1.00 | 0.00 | 0.053 | 0.00 | 0.00 |
| ATOM | 3170 | HB2 | LEU | 195 | −1.959 | 6.017 | 27.634 | 1.00 | 0.00 | 0.053 | 0.00 | 0.00 |
| ATOM | 3171 | CG | LEU | 195 | −3.695 | 6.026 | 28.842 | 1.00 | 0.00 | −0.053 | 9.40 | 4.00 |
| ATOM | 3172 | HG | LEU | 195 | −4.198 | 5.274 | 28.234 | 1.00 | 0.00 | 0.053 | 0.00 | 0.00 |
| ATOM | 3173 | CD1 | LEU | 195 | −4.129 | 5.820 | 30.300 | 1.00 | 0.00 | −0.159 | 16.15 | 4.00 |
| ATOM | 3174 | HD1 | LEU | 195 | −5.213 | 5.900 | 30.371 | 1.00 | 0.00 | 0.053 | 0.00 | 0.00 |
| ATOM | 3175 | HD1 | LEU | 195 | −3.815 | 4.831 | 30.636 | 1.00 | 0.00 | 0.053 | 0.00 | 0.00 |
| ATOM | 3176 | HD1 | LEU | 195 | −3.666 | 6.581 | 30.928 | 1.00 | 0.00 | 0.053 | 0.00 | 0.00 |
| ATOM | 3177 | CD2 | LEU | 195 | −4.234 | 7.328 | 28.241 | 1.00 | 0.00 | −0.159 | 16.15 | 4.00 |
| ATOM | 3178 | HD2 | LEU | 195 | −5.316 | 7.363 | 28.362 | 1.00 | 0.00 | 0.053 | 0.00 | 0.00 |
| ATOM | 3179 | HD2 | LEU | 195 | −3.783 | 8.178 | 28.752 | 1.00 | 0.00 | 0.053 | 0.00 | 0.00 |
| ATOM | 3180 | HD2 | LEU | 195 | −3.985 | 7.369 | 27.180 | 1.00 | 0.00 | 0.053 | 0.00 | 0.00 |
| ATOM | 3181 | N | GLU | 196 | 0.392 | 5.375 | 29.927 | 1.00 | 0.00 | −0.650 | 9.00 | −17.40 |
| ATOM | 3182 | HN | GLU | 196 | −0.372 | 4.728 | 30.167 | 1.00 | 0.00 | 0.440 | 0.00 | 0.00 |
| ATOM | 3183 | CA | GLU | 196 | 1.741 | 4.888 | 30.060 | 1.00 | 0.00 | 0.158 | 9.40 | 4.00 |
| ATOM | 3184 | HA | GLU | 196 | 2.337 | 5.553 | 30.683 | 1.00 | 0.00 | 0.053 | 0.00 | 0.00 |
| ATOM | 3185 | C | GLU | 196 | 2.413 | 4.787 | 28.719 | 1.00 | 0.00 | 0.396 | 9.82 | 4.00 |
| ATOM | 3186 | O | GLU | 196 | 3.591 | 5.119 | 28.593 | 1.00 | 0.00 | −0.396 | 8.17 | −17.40 |
| ATOM | 3187 | CB | GLU | 196 | 1.782 | 3.484 | 30.691 | 1.00 | 0.00 | −0.106 | 12.77 | 4.00 |
| ATOM | 3188 | HB1 | GLU | 196 | 1.645 | 2.753 | 29.893 | 1.00 | 0.00 | 0.053 | 0.00 | 0.00 |
| ATOM | 3189 | HB2 | GLU | 196 | 0.974 | 3.419 | 31.420 | 1.00 | 0.00 | 0.053 | 0.00 | 0.00 |
| ATOM | 3190 | CG | GLU | 196 | 3.078 | 3.123 | 31.418 | 1.00 | 0.00 | −0.106 | 12.77 | 4.00 |
| ATOM | 3191 | HG1 | GLU | 196 | 3.896 | 3.629 | 30.906 | 1.00 | 0.00 | 0.053 | 0.00 | 0.00 |
| ATOM | 3192 | HG2 | GLU | 196 | 3.193 | 2.040 | 31.370 | 1.00 | 0.00 | 0.053 | 0.00 | 0.00 |
| ATOM | 3193 | CD | GLU | 196 | 2.948 | 3.601 | 32.860 | 1.00 | 0.00 | 0.399 | 9.82 | 4.00 |
| ATOM | 3194 | OE1 | GLU | 196 | 2.274 | 4.642 | 33.079 | 1.00 | 0.00 | −0.396 | 8.17 | −18.95 |
| ATOM | 3195 | OE2 | GLU | 196 | 3.521 | 2.932 | 33.761 | 1.00 | 0.00 | −0.427 | 8.17 | −18.95 |
| ATOM | 3197 | N | THR | 197 | 1.693 | 4.324 | 27.676 | 1.00 | 0.00 | −0.650 | 9.00 | −17.40 |
| ATOM | 3198 | HN | THR | 197 | 0.689 | 4.120 | 27.789 | 1.00 | 0.00 | 0.440 | 0.00 | 0.00 |
| ATOM | 3199 | CA | THR | 197 | 2.333 | 4.115 | 26.403 | 1.00 | 0.00 | 0.158 | 9.40 | 4.00 |
| ATOM | 3200 | HA | THR | 197 | 3.411 | 4.214 | 26.525 | 1.00 | 0.00 | 0.053 | 0.00 | 0.00 |
| ATOM | 3201 | C | THR | 197 | 1.835 | 5.141 | 25.428 | 1.00 | 0.00 | 0.396 | 9.82 | 4.00 |
| ATOM | 3202 | O | THR | 197 | 0.726 | 5.655 | 25.558 | 1.00 | 0.00 | −0.396 | 8.17 | −17.40 |
| ATOM | 3203 | CB | THR | 197 | 2.017 | 2.776 | 25.796 | 1.00 | 0.00 | 0.060 | 9.40 | 4.00 |
| ATOM | 3204 | HB | THR | 197 | 0.942 | 2.705 | 25.625 | 1.00 | 0.00 | 0.053 | 0.00 | 0.00 |
| ATOM | 3205 | OG1 | THR | 197 | 2.410 | 1.735 | 26.675 | 1.00 | 0.00 | −0.537 | 11.04 | −17.40 |
| ATOM | 3206 | HG1 | THR | 197 | 1.792 | 1.729 | 27.499 | 1.00 | 0.00 | 0.424 | 0.00 | 0.00 |
| ATOM | 3207 | CG2 | THR | 197 | 2.767 | 2.638 | 24.459 | 1.00 | 0.00 | −0.159 | 16.15 | 4.00 |
| ATOM | 3208 | HG2 | THR | 197 | 2.541 | 1.668 | 24.013 | 1.00 | 0.00 | 0.053 | 0.00 | 0.00 |
| ATOM | 3209 | HG2 | TRR | 197 | 2.451 | 3.430 | 23.781 | 1.00 | 0.00 | 0.053 | 0.00 | 0.00 |
| ATOM | 3210 | HG2 | THR | 197 | 3.840 | 2.716 | 24.633 | 1.00 | 0.00 | 0.053 | 0.00 | 0.00 |
| ATOM | 3211 | N | GLU | 198 | 2.674 | 5.458 | 24.419 | 1.00 | 0.00 | −0.650 | 9.00 | −17.40 |
| ATOM | 3212 | HN | GLU | 198 | 3.576 | 4.964 | 24.369 | 1.00 | 0.00 | 0.440 | 0.00 | 0.00 |
| ATOM | 3213 | CA | GLU | 198 | 2.389 | 6.440 | 23.410 | 1.00 | 0.00 | 0.158 | 9.40 | 4.00 |
| ATOM | 3214 | HA | GLU | 198 | 1.738 | 7.215 | 23.813 | 1.00 | 0.00 | 0.053 | 0.00 | 0.00 |
| ATOM | 3215 | C | GLU | 198 | 1.707 | 5.798 | 22.244 | 1.00 | 0.00 | 0.396 | 9.82 | 4.00 |
| ATOM | 3216 | O | GLU | 198 | 2.020 | 4.667 | 21.874 | 1.00 | 0.00 | −0.396 | 8.17 | −17.40 |
| ATOM | 3217 | CB | GLU | 198 | 3.664 | 7.079 | 22.838 | 1.00 | 0.00 | −0.106 | 12.77 | 4.00 |
| ATOM | 3218 | HB1 | GLU | 198 | 4.295 | 6.286 | 22.435 | 1.00 | 0.00 | 0.053 | 0.00 | 0.00 |
| ATOM | 3219 | HB2 | GLU | 198 | 4.177 | 7.604 | 23.643 | 1.00 | 0.00 | 0.053 | 0.00 | 0.00 |
| ATOM | 3220 | CG | GLU | 198 | 3.402 | 8.083 | 21.717 | 1.00 | 0.00 | −0.106 | 12.77 | 4.00 |
| ATOM | 3221 | HG1 | GLU | 198 | 2.555 | 7.716 | 21.136 | 1.00 | 0.00 | 0.053 | 0.00 | 0.00 |
| ATOM | 3222 | HG2 | GLU | 198 | 4.303 | 8.140 | 21.107 | 1.00 | 0.00 | 0.053 | 0.00 | 0.00 |
| ATOM | 3223 | CD | GLU | 198 | 3.089 | 9.420 | 22.361 | 1.00 | 0.00 | 0.399 | 9.82 | 4.00 |
| ATOM | 3224 | OE1 | GLU | 198 | 4.038 | 10.021 | 22.930 | 1.00 | 0.00 | −0.396 | 8.17 | −18.95 |
| ATOM | 3225 | OE2 | GLU | 198 | 1.908 | 9.854 | 22.304 | 1.00 | 0.00 | −0.427 | 8.17 | −18.95 |
| ATOM | 3227 | N | ARG | 199G | 0.725 | 6.514 | 21.653 | 1.00 | 0.00 | −0.650 | 9.00 | −17.40 |
| ATOM | 3228 | HN | ARG | 199G | 0.446 | 7.417 | 22.062 | 1.00 | 0.00 | 0.440 | 0.00 | 0.00 |
| ATOM | 3229 | CA | ARG | 199G | 0.060 | 6.048 | 20.470 | 1.00 | 0.00 | 0.158 | 9.40 | 4.00 |
| ATOM | 3230 | HA | ARG | 199G | −0.267 | 5.018 | 20.614 | 1.00 | 0.00 | 0.053 | 0.00 | 0.00 |
| ATOM | 3231 | C | ARG | 199G | 1.011 | 6.109 | 19.292 | 1.00 | 0.00 | 0.396 | 9.82 | 4.00 |
| ATOM | 3232 | O | ARG | 199G | 1.042 | 5.130 | 18.546 | 1.00 | 0.00 | −0.396 | 8.17 | −17.40 |
| ATOM | 3233 | CB | ARG | 199G | −1.204 | 6.847 | 20.119 | 1.00 | 0.00 | −0.106 | 12.77 | 4.00 |
| ATOM | 3234 | HB1 | ARG | 199G | −1.566 | 6.632 | 19.113 | 1.00 | 0.00 | 0.053 | 0.00 | 0.00 |
| ATOM | 3235 | HB2 | ARG | 199G | −1.041 | 7.924 | 20.162 | 1.00 | 0.00 | 0.053 | 0.00 | 0.00 |
| ATOM | 3236 | CG | ARG | 199G | −2.366 | 6.549 | 21.067 | 1.00 | 0.00 | −0.106 | 12.77 | 4.00 |
| ATOM | 3237 | HG1 | ARG | 199G | −3.198 | 7.245 | 20.969 | 1.00 | 0.00 | 0.053 | 0.00 | 0.00 |
| ATOM | 3238 | HG2 | ARG | 199G | −2.090 | 6.584 | 22.120 | 1.00 | 0.00 | 0.053 | 0.00 | 0.00 |
| ATOM | 3239 | CD | ARG | 199G | −2.982 | 5.164 | 20.865 | 1.00 | 0.00 | 0.374 | 12.77 | 4.00 |
| ATOM | 3240 | HD1 | ARG | 199G | −3.245 | 5.068 | 19.811 | 1.00 | 0.00 | 0.053 | 0.00 | 0.00 |
| ATOM | 3241 | HD2 | ARG | 199G | −3.866 | 5.098 | 21.499 | 1.00 | 0.00 | 0.053 | 0.00 | 0.00 |
| ATOM | 3242 | NE | ARG | 199G | −1.962 | 4.153 | 21.258 | 1.00 | 0.00 | −0.819 | 9.00 | −24.67 |
| ATOM | 3243 | HE | ARG | 199G | −1.348 | 3.739 | 20.541 | 1.00 | 0.00 | 0.407 | 0.00 | 0.00 |
| ATOM | 3244 | CZ | ARG | 199G | −1.836 | 3.772 | 22.563 | 1.00 | 0.00 | 0.796 | 6.95 | 4.00 |
| ATOM | 3245 | NH1 | ARG | 199G | −2.612 | 4.352 | 23.524 | 1.00 | 0.00 | −0.746 | 9.00 | −24.67 |
| ATOM | 3246 | HH1 | ARG | 199G | −2.515 | 4.063 | 24.507 | 1.00 | 0.00 | 0.407 | 0.00 | 0.00 |
| ATOM | 3247 | HH1 | ARG | 199G | −3.294 | 5.078 | 23.265 | 1.00 | 0.00 | 0.407 | 0.00 | 0.00 |
| ATOM | 3248 | NH2 | ARG | 199G | −0.938 | 2.804 | 22.911 | 1.00 | 0.00 | −0.746 | 9.00 | −24.67 |
| ATOM | 3249 | HH2 | ARG | 199G | −0.844 | 2.518 | 23.896 | 1.00 | 0.00 | 0.407 | 0.00 | 0.00 |
| ATOM | 3250 | HH2 | ARG | 199G | −0.354 | 2.359 | 22.188 | 1.00 | 0.00 | 0.407 | 0.00 | 0.00 |
| ATOM | 3251 | N | MET | 200 | 1.765 | 7.243 | 19.078 | 1.00 | 0.00 | −0.650 | 9.00 | −17.40 |
| ATOM | 3252 | HN | MET | 200 | 1.616 | 8.048 | 19.702 | 1.00 | 0.00 | 0.440 | 0.00 | 0.00 |
| ATOM | 3253 | CA | MET | 200 | 2.764 | 7.371 | 18.016 | 1.00 | 0.00 | 0.158 | 9.40 | 4.00 |
| ATOM | 3254 | HA | MET | 200 | 3.667 | 6.794 | 18.216 | 1.00 | 0.00 | 0.053 | 0.00 | 0.00 |
| ATOM | 3255 | C | MET | 200 | 3.220 | 8.811 | 17.808 | 1.00 | 0.00 | 0.396 | 9.82 | 4.00 |
| ATOM | 3256 | O | MET | 200 | 2.525 | 9.752 | 18.184 | 1.00 | 0.00 | −0.396 | 8.17 | −17.40 |
| ATOM | 3257 | CB | MET | 200 | 2.270 | 6.900 | 16.646 | 1.00 | 0.00 | −0.106 | 12.77 | 4.00 |
| ATOM | 3258 | HB1 | MET | 200 | 1.413 | 7.470 | 16.286 | 1.00 | 0.00 | 0.053 | 0.00 | 0.00 |
| ATOM | 3259 | HB2 | MET | 200 | 1.955 | 5.856 | 16.645 | 1.00 | 0.00 | 0.053 | 0.00 | 0.00 |
| ATOM | 3260 | CG | MET | 200 | 3.337 | 7.016 | 15.559 | 1.00 | 0.00 | −0.041 | 12.77 | 4.00 |
| ATOM | 3261 | HG1 | MET | 200 | 3.769 | 8.015 | 15.612 | 1.00 | 0.00 | 0.053 | 0.00 | 0.00 |
| ATOM | 3262 | HG2 | MET | 200 | 2.857 | 6.854 | 14.593 | 1.00 | 0.00 | 0.053 | 0.00 | 0.00 |
| ATOM | 3263 | SD | MET | 200 | 4.700 | 5.823 | 15.698 | 1.00 | 0.00 | −0.130 | 16.39 | −6.40 |
| ATOM | 3264 | CE | MET | 200 | 5.576 | 6.428 | 14.227 | 1.00 | 0.00 | −0.094 | 16.15 | 4.00 |
| ATOM | 3265 | HE1 | MET | 200 | 6.484 | 5.844 | 14.078 | 1.00 | 0.00 | 0.053 | 0.00 | 0.00 |
| ATOM | 3266 | HE2 | MET | 200 | 4.932 | 6.326 | 13.352 | 1.00 | 0.00 | 0.053 | 0.00 | 0.00 |
| ATOM | 3267 | HE3 | MET | 200 | 5.837 | 7.477 | 14.364 | 1.00 | 0.00 | 0.053 | 0.00 | 0.00 |
| ATOM | 3268 | N | PRO | 201 | 4.395 | 8.995 | 17.204 | 1.00 | 0.00 | −0.422 | 9.00 | −17.40 |
| ATOM | 3269 | CA | PRO | 201 | 4.871 | 10.354 | 16.944 | 1.00 | 0.00 | 0.158 | 9.40 | 4.00 |
| ATOM | 3270 | HA | PRO | 201 | 4.120 | 11.103 | 17.195 | 1.00 | 0.00 | 0.053 | 0.00 | 0.00 |
| ATOM | 3271 | CD | PRO | 201 | 5.465 | 8.197 | 17.788 | 1.00 | 0.00 | 0.105 | 12.77 | 4.00 |
| ATOM | 3272 | HD1 | PRO | 201 | 5.605 | 7.340 | 17.129 | 1.00 | 0.00 | 0.053 | 0.00 | 0.00 |
| ATOM | 3273 | HD2 | PRO | 201 | 5.124 | 7.904 | 18.781 | 1.00 | 0.00 | 0.053 | 0.00 | 0.00 |
| ATOM | 3274 | C | PRO | 201 | 5.227 | 10.559 | 15.486 | 1.00 | 0.00 | 0.396 | 9.82 | 4.00 |
| ATOM | 3275 | O | PRO | 201 | 5.295 | 9.570 | 14.763 | 1.00 | 0.00 | −0.396 | 8.17 | −17.40 |
| ATOM | 3276 | CB | PRO | 201 | 6.121 | 10.522 | 17.801 | 1.00 | 0.00 | −0.106 | 12.77 | 4.00 |
| ATOM | 3277 | HB1 | PRO | 201 | 5.868 | 10.885 | 18.797 | 1.00 | 0.00 | 0.053 | 0.00 | 0.00 |
| ATOM | 3278 | HB2 | PRO | 201 | 6.812 | 11.234 | 17.352 | 1.00 | 0.00 | 0.053 | 0.00 | 0.00 |
| ATOM | 3279 | CG | PRO | 201 | 6.704 | 9.103 | 17.839 | 1.00 | 0.00 | −0.106 | 12.77 | 4.00 |
| ATOM | 3280 | HG1 | PRO | 201 | 7.281 | 8.943 | 18.749 | 1.00 | 0.00 | 0.053 | 0.00 | 0.00 |
| ATOM | 3281 | HG2 | PRO | 201 | 7.363 | 8.930 | 16.988 | 1.00 | 0.00 | 0.053 | 0.00 | 0.00 |
| ATOM | 3282 | N | SER | 202 | 5.485 | 11.820 | 15.035 | 1.00 | 0.00 | −0.650 | 9.00 | −17.40 |
| ATOM | 3283 | HN | SER | 202 | 5.423 | 12.606 | 15.696 | 1.00 | 0.00 | 0.440 | 0.00 | 0.00 |
| ATOM | 3284 | CA | SER | 202 | 5.843 | 12.092 | 13.657 | 1.00 | 0.00 | 0.158 | 9.40 | 4.00 |
| ATOM | 3285 | HA | SER | 202 | 5.931 | 11.158 | 13.101 | 1.00 | 0.00 | 0.053 | 0.00 | 0.00 |
| ATOM | 3286 | C | SER | 202 | 7.158 | 12.818 | 13.598 | 1.00 | 0.00 | 0.396 | 9.82 | 4.00 |
| ATOM | 3287 | O | SER | 202 | 7.321 | 13.887 | 14.186 | 1.00 | 0.00 | −0.396 | 8.17 | −17.40 |
| ATOM | 3288 | CB | SER | 202 | 4.844 | 13.002 | 12.927 | 1.00 | 0.00 | 0.007 | 12.77 | 4.00 |
| ATOM | 3289 | HB1 | SER | 202 | 3.840 | 12.580 | 12.989 | 1.00 | 0.00 | 0.053 | 0.00 | 0.00 |
| ATOM | 3290 | HB2 | SER | 202 | 5.123 | 13.096 | 11.877 | 1.00 | 0.00 | 0.053 | 0.00 | 0.00 |
| ATOM | 3291 | OG | SER | 202 | 4.839 | 14.292 | 13.520 | 1.00 | 0.00 | −0.537 | 11.04 | −17.40 |
| ATOM | 3292 | HG | SER | 202 | 5.720 | 14.776 | 13.296 | 1.00 | 0.00 | 0.424 | 0.00 | 0.00 |
| ATOM | 3293 | N | HIS | 203S | 8.134 | 12.245 | 12.861 | 1.00 | 0.00 | −0.650 | 9.00 | −17.40 |
| ATOM | 3294 | HN | HIS | 203S | 7.939 | 11.349 | 12.390 | 1.00 | 0.00 | 0.440 | 0.00 | 0.00 |
| ATOM | 3295 | CA | HIS | 203S | 9.435 | 12.839 | 12.709 | 1.00 | 0.00 | 0.158 | 9.40 | 4.00 |
| ATOM | 3296 | HA | HIS | 203S | 9.863 | 13.073 | 13.683 | 1.00 | 0.00 | 0.053 | 0.00 | 0.00 |
| ATOM | 3297 | C | HIS | 203S | 9.343 | 14.104 | 11.916 | 1.00 | 0.00 | 0.396 | 9.82 | 4.00 |
| ATOM | 3298 | O | HIS | 203S | 9.857 | 15.142 | 12.331 | 1.00 | 0.00 | −0.396 | 8.17 | −17.40 |
| ATOM | 3299 | CB | HIS | 203S | 10.429 | 11.932 | 11.962 | 1.00 | 0.00 | −0.106 | 12.77 | 4.00 |
| ATOM | 3300 | HB1 | HIS | 203S | 11.309 | 12.523 | 11.710 | 1.00 | 0.00 | 0.053 | 0.00 | 0.00 |
| ATOM | 3301 | HB2 | HIS | 203S | 9.943 | 11.563 | 11.058 | 1.00 | 0.00 | 0.053 | 0.00 | 0.00 |
| ATOM | 3302 | CG | HIS | 203S | 10.889 | 10.742 | 12.751 | 1.00 | 0.00 | −0.050 | 7.26 | 0.60 |
| ATOM | 3303 | ND1 | HIS | 203S | 10.132 | 9.614 | 12.972 | 1.00 | 0.00 | 0.207 | 9.25 | −17.40 |
| ATOM | 3304 | HD1 | HIS | 203S | 9.172 | 9.443 | 12.639 | 1.00 | 0.00 | 0.393 | 0.00 | 0.00 |
| ATOM | 3305 | CD2 | HIS | 203S | 12.079 | 10.519 | 13.374 | 1.00 | 0.00 | −0.177 | 10.80 | 0.60 |
| ATOM | 3306 | HD2 | HIS | 203S | 12.911 | 11.222 | 13.391 | 1.00 | 0.00 | 0.127 | 0.00 | 0.00 |
| ATOM | 3307 | CE1 | HIS | 203S | 10.894 | 8.770 | 13.712 | 1.00 | 0.00 | −0.227 | 10.80 | 0.60 |
| ATOM | 3308 | HE1 | HIS | 203S | 10.562 | 7.787 | 14.046 | 1.00 | 0.00 | 0.127 | 0.00 | 0.00 |
| ATOM | 3309 | NE2 | HIS | 203S | 12.084 | 9.276 | 13.981 | 1.00 | 0.00 | 0.207 | 9.25 | −17.40 |
| ATOM | 3310 | HE2 | HIS | 203S | 12.845 | 8.842 | 14.521 | 1.00 | 0.00 | 0.393 | 0.00 | 0.00 |
| ATOM | 3311 | N | LYS | 204S | 8.665 | 14.062 | 10.753 | 1.00 | 0.00 | −0.650 | 9.00 | −17.40 |
| ATOM | 3312 | HN | LYS | 204S | 8.179 | 13.202 | 10.459 | 1.00 | 0.00 | 0.440 | 0.00 | 0.00 |
| ATOM | 3313 | CA | LYS | 204S | 8.632 | 15.236 | 9.933 | 1.00 | 0.00 | 0.158 | 9.40 | 4.00 |
| ATOM | 3314 | HA | LYS | 204S | 9.381 | 15.929 | 10.314 | 1.00 | 0.00 | 0.053 | 0.00 | 0.00 |
| ATOM | 3315 | C | LYS | 204S | 7.267 | 15.818 | 10.027 | 1.00 | 0.00 | 0.396 | 9.82 | 4.00 |
| ATOM | 3316 | O | LYS | 204S | 6.270 | 15.099 | 10.034 | 1.00 | 0.00 | −0.396 | 8.17 | −17.40 |
| ATOM | 3317 | CB | LYS | 204S | 8.960 | 14.968 | 8.449 | 1.00 | 0.00 | −0.106 | 12.77 | 4.00 |
| ATOM | 3318 | HB1 | LYS | 204S | 9.911 | 14.438 | 8.403 | 1.00 | 0.00 | 0.053 | 0.00 | 0.00 |
| ATOM | 3319 | HB2 | LYS | 204S | 9.026 | 15.928 | 7.938 | 1.00 | 0.00 | 0.053 | 0.00 | 0.00 |
| ATOM | 3320 | CG | LYS | 204S | 7.930 | 14.121 | 7.694 | 1.00 | 0.00 | −0.106 | 12.77 | 4.00 |
| ATOM | 3321 | HG1 | LYS | 204S | 7.633 | 13.289 | 8.333 | 1.00 | 0.00 | 0.053 | 0.00 | 0.00 |
| ATOM | 3322 | HG2 | LYS | 204S | 8.392 | 13.752 | 6.778 | 1.00 | 0.00 | 0.053 | 0.00 | 0.00 |
| ATOM | 3323 | CD | LYS | 204S | 6.663 | 14.885 | 7.303 | 1.00 | 0.00 | −0.106 | 12.77 | 4.00 |
| ATOM | 3324 | HD1 | LYS | 204S | 6.176 | 15.376 | 8.145 | 1.00 | 0.00 | 0.053 | 0.00 | 0.00 |
| ATOM | 3325 | HD2 | LYS | 204S | 5.895 | 14.252 | 6.857 | 1.00 | 0.00 | 0.053 | 0.00 | 0.00 |
| ATOM | 3326 | CE | LYS | 204S | 6.913 | 15.995 | 6.279 | 1.00 | 0.00 | 0.099 | 12.77 | 4.00 |
| ATOM | 3327 | HE1 | LYS | 204S | 7.324 | 15.573 | 5.362 | 1.00 | 0.00 | 0.053 | 0.00 | 0.00 |
| ATOM | 3328 | HE2 | LYS | 204S | 7.619 | 16.722 | 6.679 | 1.00 | 0.00 | 0.053 | 0.00 | 0.00 |
| ATOM | 3329 | NZ | LYS | 204S | 5.645 | 16.687 | 5.959 | 1.00 | 0.00 | −0.045 | 13.25 | −39.20 |
| ATOM | 3330 | HZ1 | LYS | 204S | 5.823 | 17.431 | 5.269 | 1.00 | 0.00 | 0.280 | 0.00 | 0.00 |
| ATOM | 3331 | HZ2 | LYS | 204S | 4.973 | 16.011 | 5.567 | 1.00 | 0.00 | 0.280 | 0.00 | 0.00 |
| ATOM | 3332 | HZ3 | LYS | 204S | 5.253 | 17.101 | 6.816 | 1.00 | 0.00 | 0.280 | 0.00 | 0.00 |
| ATOM | 3333 | N | GLU | 205 | 7.195 | 17.157 | 10.115 | 1.00 | 0.00 | −0.650 | 9.00 | −17.40 |
| ATOM | 3334 | HN | GLU | 205 | 8.053 | 17.725 | 10.084 | 1.00 | 0.00 | 0.440 | 0.00 | 0.00 |
| ATOM | 3335 | CA | GLU | 205 | 5.921 | 17.787 | 10.251 | 1.00 | 0.00 | 0.158 | 9.40 | 4.00 |
| ATOM | 3336 | HA | GLU | 205 | 5.229 | 17.236 | 9.613 | 1.00 | 0.00 | 0.053 | 0.00 | 0.00 |
| ATOM | 3337 | C | GLU | 205 | 6.091 | 19.200 | 9.804 | 1.00 | 0.00 | 0.396 | 9.82 | 4.00 |
| ATOM | 3338 | O | GLU | 205 | 7.048 | 19.541 | 9.112 | 1.00 | 0.00 | −0.396 | 8.17 | −17.40 |
| ATOM | 3339 | CB | GLU | 205 | 5.436 | 17.791 | 11.712 | 1.00 | 0.00 | −0.106 | 12.77 | 4.00 |
| ATOM | 3340 | HB1 | GLU | 205 | 4.703 | 18.590 | 11.822 | 1.00 | 0.00 | 0.053 | 0.00 | 0.00 |
| ATOM | 3341 | HB2 | GLU | 205 | 6.299 | 17.965 | 12.354 | 1.00 | 0.00 | 0.053 | 0.00 | 0.00 |
| ATOM | 3342 | CG | GLU | 205 | 4.772 | 16.495 | 12.182 | 1.00 | 0.00 | −0.106 | 12.77 | 4.00 |
| ATOM | 3343 | HG1 | GLU | 205 | 5.499 | 15.694 | 12.050 | 1.00 | 0.00 | 0.053 | 0.00 | 0.00 |
| ATOM | 3344 | HG2 | GLU | 205 | 3.888 | 16.342 | 11.561 | 1.00 | 0.00 | 0.053 | 0.00 | 0.00 |
| ATOM | 3345 | CD | GLU | 205 | 4.404 | 16.684 | 13.647 | 1.00 | 0.00 | 0.399 | 9.82 | 4.00 |
| ATOM | 3346 | OE1 | GLU | 205 | 5.329 | 16.611 | 14.500 | 1.00 | 0.00 | −0.396 | 8.17 | −18.95 |
| ATOM | 3347 | OE2 | GLU | 205 | 3.197 | 16.904 | 13.934 | 1.00 | 0.00 | −0.427 | 8.17 | −18.95 |
| ATOM | 3349 | N | GLU | 206 | 5.118 | 20.052 | 10.161 | 1.00 | 0.00 | −0.650 | 9.00 | −17.40 |
| ATOM | 3350 | HN | GLU | 206 | 4.306 | 19.698 | 10.687 | 1.00 | 0.00 | 0.440 | 0.00 | 0.00 |
| ATOM | 3351 | CA | GLU | 206 | 5.184 | 21.439 | 9.827 | 1.00 | 0.00 | 0.158 | 9.40 | 4.00 |
| ATOM | 3352 | HA | GLU | 206 | 5.300 | 21.500 | 8.745 | 1.00 | 0.00 | 0.053 | 0.00 | 0.00 |
| ATOM | 3353 | C | GLU | 206 | 6.365 | 22.005 | 10.547 | 1.00 | 0.00 | 0.396 | 9.82 | 4.00 |
| ATOM | 3354 | O | GLU | 206 | 7.167 | 22.741 | 9.974 | 1.00 | 0.00 | −0.396 | 8.17 | −17.40 |
| ATOM | 3355 | CB | GLU | 206 | 3.923 | 22.184 | 10.303 | 1.00 | 0.00 | −0.106 | 12.77 | 4.00 |
| ATOM | 3356 | HB1 | GLU | 206 | 3.866 | 22.096 | 11.388 | 1.00 | 0.00 | 0.053 | 0.00 | 0.00 |
| ATOM | 3357 | HB2 | GLU | 206 | 3.056 | 21.720 | 9.832 | 1.00 | 0.00 | 0.053 | 0.00 | 0.00 |
| ATOM | 3358 | CG | GLU | 206 | 3.888 | 23.673 | 9.961 | 1.00 | 0.00 | −0.106 | 12.77 | 4.00 |
| ATOM | 3359 | HG1 | GLU | 206 | 2.855 | 24.019 | 10.009 | 1.00 | 0.00 | 0.053 | 0.00 | 0.00 |
| ATOM | 3360 | HG2 | GLU | 206 | 4.283 | 23.811 | 8.954 | 1.00 | 0.00 | 0.053 | 0.00 | 0.00 |
| ATOM | 3361 | CD | GLU | 206 | 4.743 | 24.427 | 10.969 | 1.00 | 0.00 | 0.399 | 9.82 | 4.00 |
| ATOM | 3362 | OE1 | GLU | 206 | 4.525 | 24.234 | 12.194 | 1.00 | 0.00 | −0.396 | 8.17 | −18.95 |
| ATOM | 3363 | OE2 | GLU | 206 | 5.632 | 25.200 | 10.523 | 1.00 | 0.00 | −0.427 | 8.17 | −18.95 |
| ATOM | 3365 | N | ARG | 207G | 6.512 | 21.644 | 11.836 | 1.00 | 0.00 | −0.650 | 9.00 | −17.40 |
| ATOM | 3366 | HN | ARG | 207G | 5.839 | 20.988 | 12.260 | 1.00 | 0.00 | 0.440 | 0.00 | 0.00 |
| ATOM | 3367 | CA | ARG | 207G | 7.590 | 22.159 | 12.625 | 1.00 | 0.00 | 0.158 | 9.40 | 4.00 |
| ATOM | 3368 | HA | ARG | 207G | 7.638 | 23.237 | 12.473 | 1.00 | 0.00 | 0.053 | 0.00 | 0.00 |
| ATOM | 3369 | C | ARG | 207G | 8.851 | 21.506 | 12.173 | 1.00 | 0.00 | 0.396 | 9.82 | 4.00 |
| ATOM | 3370 | O | ARG | 207G | 8.833 | 20.571 | 11.373 | 1.00 | 0.00 | −0.396 | 8.17 | −17.40 |
| ATOM | 3371 | CB | ARG | 207G | 7.432 | 21.887 | 14.130 | 1.00 | 0.00 | −0.106 | 12.77 | 4.00 |
| ATOM | 3372 | HB1 | ARG | 207G | 6.524 | 22.315 | 14.554 | 1.00 | 0.00 | 0.053 | 0.00 | 0.00 |
| ATOM | 3373 | HB2 | ARG | 207G | 8.247 | 22.289 | 14.731 | 1.00 | 0.00 | 0.053 | 0.00 | 0.00 |
| ATOM | 3374 | CG | ARG | 207G | 7.374 | 20.398 | 14.476 | 1.00 | 0.00 | −0.106 | 12.77 | 4.00 |
| ATOM | 3375 | HG1 | ARG | 207G | 7.862 | 19.758 | 13.740 | 1.00 | 0.00 | 0.053 | 0.00 | 0.00 |
| ATOM | 3376 | HG2 | ARG | 207G | 6.359 | 20.007 | 14.559 | 1.00 | 0.00 | 0.053 | 0.00 | 0.00 |
| ATOM | 3377 | CD | ARG | 207G | 8.042 | 20.050 | 15.808 | 1.00 | 0.00 | 0.374 | 12.77 | 4.00 |
| ATOM | 3378 | HD1 | ARG | 207G | 7.880 | 18.987 | 15.989 | 1.00 | 0.00 | 0.053 | 0.00 | 0.00 |
| ATOM | 3379 | HD2 | ARG | 207G | 7.573 | 20.659 | 16.580 | 1.00 | 0.00 | 0.053 | 0.00 | 0.00 |
| ATOM | 3380 | NE | ARG | 207G | 9.493 | 20.362 | 15.679 | 1.00 | 0.00 | −0.819 | 9.00 | −24.67 |
| ATOM | 3381 | HE | ARG | 207G | 10.014 | 20.055 | 14.845 | 1.00 | 0.00 | 0.407 | 0.00 | 0.00 |
| ATOM | 3382 | CZ | ARG | 207G | 10.130 | 21.055 | 16.668 | 1.00 | 0.00 | 0.796 | 6.95 | 4.00 |
| ATOM | 3383 | NH1 | ARG | 207G | 9.437 | 21.460 | 17.772 | 1.00 | 0.00 | −0.746 | 9.00 | −24.67 |
| ATOM | 3384 | HH1 | ARG | 207G | 9.917 | 21.982 | 18.518 | 1.00 | 0.00 | 0.407 | 0.00 | 0.00 |
| ATOM | 3385 | HH1 | ARG | 207G | 8.433 | 21.242 | 17.858 | 1.00 | 0.00 | 0.407 | 0.00 | 0.00 |
| ATOM | 3386 | NH2 | ARG | 207G | 11.459 | 21.344 | 16.552 | 1.00 | 0.00 | −0.746 | 9.00 | −24.67 |
| ATOM | 3387 | HH2 | ARG | 207G | 11.939 | 21.866 | 17.298 | 1.00 | 0.00 | 0.407 | 0.00 | 0.00 |
| ATOM | 3388 | HH2 | ARG | 207G | 11.981 | 21.039 | 15.718 | 1.00 | 0.00 | 0.407 | 0.00 | 0.00 |
| ATOM | 3389 | N | TRP | 208 | 9.990 | 22.014 | 12.678 | 1.00 | 0.00 | −0.650 | 9.00 | −17.40 |
| ATOM | 3390 | HN | TRP | 208 | 9.938 | 22.787 | 13.355 | 1.00 | 0.00 | 0.440 | 0.00 | 0.00 |
| ATOM | 3391 | CA | TRP | 208 | 11.271 | 21.504 | 12.295 | 1.00 | 0.00 | 0.158 | 9.40 | 4.00 |
| ATOM | 3392 | HA | TRP | 208 | 11.288 | 21.348 | 11.216 | 1.00 | 0.00 | 0.053 | 0.00 | 0.00 |
| ATOM | 3393 | C | TRP | 208 | 11.514 | 20.205 | 12.995 | 1.00 | 0.00 | 0.396 | 9.82 | 4.00 |
| ATOM | 3394 | O | TRP | 208 | 10.968 | 19.935 | 14.064 | 1.00 | 0.00 | −0.396 | 8.17 | −17.40 |
| ATOM | 3395 | CB | TRP | 208 | 12.436 | 22.449 | 12.636 | 1.00 | 0.00 | −0.106 | 12.77 | 4.00 |
| ATOM | 3396 | HB1 | TRP | 208 | 13.371 | 21.920 | 12.453 | 1.00 | 0.00 | 0.053 | 0.00 | 0.00 |
| ATOM | 3397 | HB2 | TRP | 208 | 12.354 | 22.730 | 13.686 | 1.00 | 0.00 | 0.053 | 0.00 | 0.00 |
| ATOM | 3398 | CG | TRP | 208 | 12.456 | 23.719 | 11.819 | 1.00 | 0.00 | 0.000 | 7.26 | 0.60 |
| ATOM | 3399 | CD1 | TRP | 208 | 11.606 | 24.131 | 10.834 | 1.00 | 0.00 | −0.177 | 10.80 | 0.60 |
| ATOM | 3400 | HD1 | TRP | 208 | 10.739 | 23.571 | 10.481 | 1.00 | 0.00 | 0.127 | 0.00 | 0.00 |
| ATOM | 3401 | CD2 | TRP | 208 | 13.443 | 24.751 | 11.962 | 1.00 | 0.00 | 0.000 | 6.80 | 0.60 |
| ATOM | 3402 | NE1 | TRP | 208 | 12.004 | 25.355 | 10.353 | 1.00 | 0.00 | −0.292 | 9.00 | −17.40 |
| ATOM | 3403 | HE1 | TRP | 208 | 11.537 | 25.888 | 9.605 | 1.00 | 0.00 | 0.393 | 0.00 | 0.00 |
| ATOM | 3404 | CE2 | TRP | 208 | 13.133 | 25.749 | 11.039 | 1.00 | 0.00 | −0.050 | 6.80 | 0.60 |
| ATOM | 3405 | CE3 | TRP | 208 | 14.520 | 24.858 | 12.794 | 1.00 | 0.00 | −0.127 | 10.80 | 0.60 |
| ATOM | 3406 | HE3 | TRP | 208 | 14.760 | 24.075 | 13.514 | 1.00 | 0.00 | 0.127 | 0.00 | 0.00 |
| ATOM | 3407 | CZ2 | TRP | 208 | 13.900 | 26.875 | 10.933 | 1.00 | 0.00 | −0.127 | 10.80 | 0.60 |
| ATOM | 3408 | HZ2 | TRP | 208 | 13.661 | 27.656 | 10.211 | 1.00 | 0.00 | 0.127 | 0.00 | 0.00 |
| ATOM | 3409 | CZ3 | TRP | 208 | 15.291 | 25.994 | 12.687 | 1.00 | 0.00 | −0.127 | 10.80 | 0.60 |
| ATOM | 3410 | HZ3 | TRP | 208 | 16.158 | 26.114 | 13.336 | 1.00 | 0.00 | 0.127 | 0.00 | 0.00 |
| ATOM | 3411 | CH2 | TRP | 208 | 14.987 | 26.983 | 11.774 | 1.00 | 0.00 | −0.127 | 10.80 | 0.60 |
| ATOM | 3412 | HH2 | TRP | 208 | 15.619 | 27.868 | 11.716 | 1.00 | 0.00 | 0.127 | 0.00 | 0.00 |
| ATOM | 3413 | N | LYS | 209S | 12.348 | 19.363 | 12.356 | 1.00 | 0.00 | −0.650 | 9.00 | −17.40 |
| ATOM | 3414 | HN | LYS | 209S | 12.749 | 19.698 | 11.468 | 1.00 | 0.00 | 0.440 | 0.00 | 0.00 |
| ATOM | 3415 | CA | LYS | 209S | 12.738 | 18.049 | 12.778 | 1.00 | 0.00 | 0.158 | 9.40 | 4.00 |
| ATOM | 3416 | HA | LYS | 209S | 11.871 | 17.450 | 13.059 | 1.00 | 0.00 | 0.053 | 0.00 | 0.00 |
| ATOM | 3417 | C | LYS | 209S | 13.653 | 18.108 | 13.963 | 1.00 | 0.00 | 0.396 | 9.82 | 4.00 |
| ATOM | 3418 | O | LYS | 209S | 13.708 | 17.164 | 14.745 | 1.00 | 0.00 | −0.396 | 8.17 | −17.40 |
| ATOM | 3419 | CB | LYS | 209S | 13.468 | 17.271 | 11.668 | 1.00 | 0.00 | −0.106 | 12.77 | 4.00 |
| ATOM | 3420 | HB1 | LYS | 209S | 14.412 | 17.72 | 11.364 | 1.00 | 0.00 | 0.053 | 0.00 | 0.00 |
| ATOM | 3421 | HB2 | LYS | 209S | 12.893 | 17.177 | 10.746 | 1.00 | 0.00 | 0.053 | 0.00 | 0.00 |
| ATOM | 3422 | CG | LYS | 209S | 13.825 | 15.836 | 12.055 | 1.00 | 0.00 | −0.106 | 12.77 | 4.00 |
| ATOM | 3423 | HG1 | LYS | 209S | 13.005 | 15.297 | 12.531 | 1.00 | 0.00 | 0.053 | 0.00 | 0.00 |
| ATOM | 3424 | HG2 | LYS | 209S | 14.656 | 15.769 | 12.756 | 1.00 | 0.00 | 0.053 | 0.00 | 0.00 |
| ATOM | 3425 | CD | LYS | 209S | 14.236 | 14.966 | 10.863 | 1.00 | 0.00 | −0.106 | 12.77 | 4.00 |
| ATOM | 3426 | HD1 | LYS | 209S | 13.544 | 15.020 | 10.022 | 1.00 | 0.00 | 0.053 | 0.00 | 0.00 |
| ATOM | 3427 | HD2 | LYS | 209S | 14.307 | 13.904 | 11.100 | 1.00 | 0.00 | 0.053 | 0.00 | 0.00 |
| ATOM | 3428 | CE | LYS | 209S | 15.601 | 15.333 | 10.276 | 1.00 | 0.00 | 0.099 | 12.77 | 4.00 |
| ATOM | 3429 | HE1 | LYS | 209S | 16.375 | 15.222 | 11.035 | 1.00 | 0.00 | 0.053 | 0.00 | 0.00 |
| ATOM | 3430 | HE2 | LYS | 209S | 15.591 | 16.365 | 9.927 | 1.00 | 0.00 | 0.053 | 0.00 | 0.00 |
| ATOM | 3431 | NZ | LYS | 209S | 15.917 | 14.444 | 9.135 | 1.00 | 0.00 | −0.045 | 13.25 | −39.20 |
| ATOM | 3432 | HZ1 | LYS | 209S | 16.836 | 14.697 | 8.746 | 1.00 | 0.00 | 0.280 | 0.00 | 0.00 |
| ATOM | 3433 | HZ2 | LYS | 209S | 15.938 | 13.465 | 9.457 | 1.00 | 0.00 | 0.280 | 0.00 | 0.00 |
| ATOM | 3434 | HZ3 | LYS | 209S | 15.195 | 14.549 | 8.407 | 1.00 | 0.00 | 0.280 | 0.00 | 0.00 |
| ATOM | 3435 | N | LYS | 210S | 14.401 | 19.212 | 14.134 | 1.00 | 0.00 | −0.650 | 9.00 | −17.40 |
| ATOM | 3436 | HN | LYS | 210S | 14.235 | 20.031 | 13.532 | 1.00 | 0.00 | 0.440 | 0.00 | 0.00 |
| ATOM | 3437 | CA | LYS | 210S | 15.428 | 19.273 | 15.139 | 1.00 | 0.00 | 0.158 | 9.40 | 4.00 |
| ATOM | 3438 | HA | LYS | 210S | 16.232 | 18.567 | 14.932 | 1.00 | 0.00 | 0.053 | 0.00 | 0.00 |
| ATOM | 3439 | C | LYS | 210S | 14.887 | 18.948 | 16.499 | 1.00 | 0.00 | 0.396 | 9.82 | 4.00 |
| ATOM | 3440 | O | LYS | 210S | 15.469 | 18.140 | 17.221 | 1.00 | 0.00 | −0.396 | 8.17 | −17.40 |
| ATOM | 3441 | CB | LYS | 210S | 16.070 | 20.670 | 15.222 | 1.00 | 0.00 | −0.106 | 12.77 | 4.00 |
| ATOM | 3442 | HB1 | LYS | 210S | 15.290 | 21.390 | 15.469 | 1.00 | 0.00 | 0.053 | 0.00 | 0.00 |
| ATOM | 3443 | HB2 | LYS | 210S | 16.512 | 20.901 | 14.253 | 1.00 | 0.00 | 0.053 | 0.00 | 0.00 |
| ATOM | 3444 | CG | LYS | 210S | 17.171 | 20.796 | 16.277 | 1.00 | 0.00 | −0.106 | 12.77 | 4.00 |
| ATOM | 3445 | HG1 | LYS | 210S | 17.899 | 19.986 | 16.234 | 1.00 | 0.00 | 0.053 | 0.00 | 0.00 |
| ATOM | 3446 | HG2 | LYS | 210S | 16.788 | 20.792 | 17.297 | 1.00 | 0.00 | 0.053 | 0.00 | 0.00 |
| ATOM | 3447 | CD | LYS | 210S | 17.983 | 22.087 | 16.147 | 1.00 | 0.00 | −0.106 | 12.77 | 4.00 |
| ATOM | 3448 | HD1 | LYS | 210S | 17.384 | 22.993 | 16.234 | 1.00 | 0.00 | 0.053 | 0.00 | 0.00 |
| ATOM | 3449 | HD2 | LYS | 210S | 18.501 | 22.186 | 15.193 | 1.00 | 0.00 | 0.053 | 0.00 | 0.00 |
| ATOM | 3450 | CE | LYS | 210S | 19.078 | 22.237 | 17.205 | 1.00 | 0.00 | 0.099 | 12.77 | 4.00 |
| ATOM | 3451 | HE1 | LYS | 210S | 19.783 | 21.409 | 17.130 | 1.00 | 0.00 | 0.053 | 0.00 | 0.00 |
| ATOM | 3452 | HE2 | LYS | 210S | 18.635 | 22.236 | 18.201 | 1.00 | 0.00 | 0.053 | 0.00 | 0.00 |
| ATOM | 3453 | NZ | LYS | 210S | 19.806 | 23.510 | 17.002 | 1.00 | 0.00 | −0.045 | 13.25 | −39.20 |
| ATOM | 3454 | HZ1 | LYS | 210S | 20.540 | 23.603 | 17.717 | 1.00 | 0.00 | 0.280 | 0.00 | 0.00 |
| ATOM | 3455 | HZ2 | LYS | 210S | 20.236 | 23.514 | 16.066 | 1.00 | 0.00 | 0.280 | 0.00 | 0.00 |
| ATOM | 3456 | HZ3 | LYS | 210S | 19.148 | 24.298 | 17.080 | 1.00 | 0.00 | 0.280 | 0.00 | 0.00 |
| ATOM | 3457 | N | ARG | 211G | 13.750 | 19.542 | 16.894 | 1.00 | 0.00 | −0.650 | 9.00 | −17.40 |
| ATOM | 3458 | HN | ARG | 211G | 13.236 | 20.166 | 16.255 | 1.00 | 0.00 | 0.440 | 0.00 | 0.00 |
| ATOM | 3459 | CA | ARG | 211G | 13.261 | 19.294 | 18.219 | 1.00 | 0.00 | 0.158 | 9.40 | 4.00 |
| ATOM | 3460 | HA | ARG | 211G | 14.028 | 19.593 | 18.932 | 1.00 | 0.00 | 0.053 | 0.00 | 0.00 |
| ATOM | 3461 | C | ARG | 211G | 12.967 | 17.833 | 18.351 | 1.00 | 0.00 | 0.396 | 9.82 | 4.00 |
| ATOM | 3462 | O | ARG | 211G | 13.325 | 17.200 | 19.343 | 1.00 | 0.00 | −0.396 | 8.17 | −17.40 |
| ATOM | 3463 | CB | ARG | 211G | 11.964 | 20.066 | 18.511 | 1.00 | 0.00 | −0.106 | 12.77 | 4.00 |
| ATOM | 3464 | HB1 | ARG | 211G | 11.177 | 19.669 | 17.869 | 1.00 | 0.00 | 0.053 | 0.00 | 0.00 |
| ATOM | 3465 | HB2 | ARG | 211G | 12.137 | 21.120 | 18.297 | 1.00 | 0.00 | 0.053 | 0.00 | 0.00 |
| ATOM | 3466 | CG | ARG | 211G | 11.483 | 19.957 | 19.958 | 1.00 | 0.00 | −0.106 | 12.77 | 4.00 |
| ATOM | 3467 | HG1 | ARG | 211G | 11.418 | 18.931 | 20.322 | 1.00 | 0.00 | 0.053 | 0.00 | 0.00 |
| ATOM | 3468 | HG2 | ARG | 211G | 10.490 | 20.376 | 20.122 | 1.00 | 0.00 | 0.053 | 0.00 | 0.00 |
| ATOM | 3469 | CD | ARG | 211G | 12.386 | 20.675 | 20.962 | 1.00 | 0.00 | 0.374 | 12.77 | 4.00 |
| ATOM | 3470 | HD1 | ARG | 211G | 12.446 | 21.720 | 20.658 | 1.00 | 0.00 | 0.053 | 0.00 | 0.00 |
| ATOM | 3471 | HD2 | ARG | 211G | 13.362 | 20.192 | 20.925 | 1.00 | 0.00 | 0.053 | 0.00 | 0.00 |
| ATOM | 3472 | NE | ARG | 211G | 11.759 | 20.534 | 22.307 | 1.00 | 0.00 | −0.819 | 9.00 | −24.67 |
| ATOM | 3473 | HE | ARG | 211G | 11.142 | 21.276 | 22.667 | 1.00 | 0.00 | 0.407 | 0.00 | 0.00 |
| ATOM | 3474 | CZ | ARG | 211G | 12.001 | 19.423 | 23.061 | 1.00 | 0.00 | 0.796 | 6.95 | 4.00 |
| ATOM | 3475 | NE1 | ARG | 211G | 12.819 | 18.440 | 22.585 | 1.00 | 0.00 | −0.746 | 9.00 | −24.67 |
| ATOM | 3476 | HH1 | ARG | 211G | 13.001 | 17.601 | 23.154 | 1.00 | 0.00 | 0.407 | 0.00 | 0.00 |
| ATOM | 3477 | HH1 | ARG | 211G | 13.253 | 18.536 | 21.656 | 1.00 | 0.00 | 0.407 | 0.00 | 0.00 |
| ATOM | 3478 | NH2 | ARG | 211G | 11.422 | 19.295 | 24.290 | 1.00 | 0.00 | −0.746 | 9.00 | −24.67 |
| ATOM | 3479 | HH2 | ARG | 211G | 11.603 | 18.456 | 24.860 | 1.00 | 0.00 | 0.407 | 0.00 | 0.00 |
| ATOM | 3480 | HH2 | ARG | 211G | 10.803 | 20.036 | 24.647 | 1.00 | 0.00 | 0.407 | 0.00 | 0.00 |
| ATOM | 3481 | N | TYR | 212 | 12.314 | 17.254 | 17.331 | 1.00 | 0.00 | −0.650 | 9.00 | −17.40 |
| ATOM | 3482 | HN | TYR | 212 | 12.083 | 17.813 | 16.497 | 1.00 | 0.00 | 0.440 | 0.00 | 0.00 |
| ATOM | 3483 | CA | TYR | 212 | 11.931 | 15.875 | 17.376 | 1.00 | 0.00 | 0.158 | 9.40 | 4.00 |
| ATOM | 3484 | HA | TYR | 212 | 11.329 | 15.699 | 18.268 | 1.00 | 0.00 | 0.053 | 0.00 | 0.00 |
| ATOM | 3485 | C | TYR | 212 | 13.163 | 15.024 | 17.420 | 1.00 | 0.00 | 0.396 | 9.82 | 4.00 |
| ATOM | 3486 | O | TYR | 212 | 13.263 | 14.099 | 18.225 | 1.00 | 0.00 | −0.396 | 8.17 | −17.40 |
| ATOM | 3487 | CB | TYR | 212 | 11.132 | 15.462 | 16.128 | 1.00 | 0.00 | −0.106 | 12.77 | 4.00 |
| ATOM | 3488 | HB1 | TYR | 212 | 11.772 | 15.587 | 15.255 | 1.00 | 0.00 | 0.053 | 0.00 | 0.00 |
| ATOM | 3489 | HB2 | TYR | 212 | 10.254 | 16.104 | 16.056 | 1.00 | 0.00 | 0.053 | 0.00 | 0.00 |
| ATOM | 3490 | CG | TYR | 212 | 10.729 | 14.037 | 16.283 | 1.00 | 0.00 | 0.000 | 7.26 | 0.60 |
| ATOM | 3491 | CD1 | TYR | 212 | 9.549 | 13.718 | 16.915 | 1.00 | 0.00 | −0.127 | 10.80 | 0.60 |
| ATOM | 3492 | HD1 | TYR | 212 | 8.912 | 14.514 | 17.301 | 1.00 | 0.00 | 0.127 | 0.00 | 0.00 |
| ATOM | 3493 | CD2 | TYR | 212 | 11.526 | 13.023 | 15.806 | 1.00 | 0.00 | −0.127 | 10.80 | 0.60 |
| ATOM | 3494 | HD2 | TYR | 212 | 12.465 | 13.261 | 15.307 | 1.00 | 0.00 | 0.127 | 0.00 | 0.00 |
| ATOM | 3495 | CE1 | TYR | 212 | 9.163 | 12.407 | 17.065 | 1.00 | 0.00 | −0.127 | 10.80 | 0.60 |
| ATOM | 3496 | HE1 | TYR | 212 | 8.224 | 12.167 | 17.565 | 1.00 | 0.00 | 0.127 | 0.00 | 0.00 |
| ATOM | 3497 | CE2 | TYR | 212 | 11.145 | 11.710 | 15.954 | 1.00 | 0.00 | −0.127 | 10.80 | 0.60 |
| ATOM | 3498 | HE2 | TYR | 212 | 11.782 | 10.912 | 15.571 | 1.00 | 0.00 | 0.127 | 0.00 | 0.00 |
| ATOM | 3499 | CZ | TYR | 212 | 9.963 | 11.400 | 16.583 | 1.00 | 0.00 | 0.026 | 7.26 | 0.60 |
| ATOM | 3500 | OH | TYR | 212 | 9.572 | 10.053 | 16.733 | 1.00 | 0.00 | −0.451 | 10.94 | −17.40 |
| ATOM | 3501 | HH | TYR | 212 | 8.549 | 10.002 | 16.846 | 1.00 | 0.00 | 0.424 | 0.00 | 0.00 |
| ATOM | 3502 | N | GLU | 213 | 14.148 | 15.339 | 16.558 | 1.00 | 0.00 | −0.650 | 9.00 | −17.40 |
| ATOM | 3503 | HN | GLU | 213 | 14.041 | 16.173 | 15.963 | 1.00 | 0.00 | 0.440 | 0.00 | 0.00 |
| ATOM | 3504 | CA | GLU | 213 | 15.342 | 14.551 | 16.439 | 1.00 | 0.00 | 0.158 | 9.40 | 4.00 |
| ATOM | 3505 | HA | GLU | 213 | 15.077 | 13.518 | 16.210 | 1.00 | 0.00 | 0.053 | 0.00 | 0.00 |
| ATOM | 3506 | C | GLU | 213 | 16.099 | 14.594 | 17.721 | 1.00 | 0.00 | 0.396 | 9.82 | 4.00 |
| ATOM | 3507 | O | GLU | 213 | 16.596 | 13.570 | 18.186 | 1.00 | 0.00 | −0.396 | 8.17 | −17.40 |
| ATOM | 3508 | CB | GLU | 213 | 16.279 | 15.034 | 15.315 | 1.00 | 0.00 | −0.106 | 12.77 | 4.00 |
| ATOM | 3509 | HB1 | GLU | 213 | 15.729 | 15.006 | 14.374 | 1.00 | 0.00 | 0.053 | 0.00 | 0.00 |
| ATOM | 3510 | HB2 | GLU | 213 | 17.140 | 14.366 | 15.275 | 1.00 | 0.00 | 0.053 | 0.00 | 0.00 |
| ATOM | 3511 | CG | GLU | 213 | 16.801 | 16.459 | 15.509 | 1.00 | 0.00 | −0.106 | 12.77 | 4.00 |
| ATOM | 3512 | HG1 | GLU | 213 | 17.357 | 16.488 | 16.445 | 1.00 | 0.00 | 0.053 | 0.00 | 0.00 |
| ATOM | 3513 | HG2 | GLU | 213 | 15.942 | 17.129 | 15.544 | 1.00 | 0.00 | 0.053 | 0.00 | 0.00 |
| ATOM | 3514 | CD | GLU | 213 | 17.704 | 16.793 | 14.329 | 1.00 | 0.00 | 0.399 | 9.82 | 4.00 |
| ATOM | 3515 | OE1 | GLU | 213 | 17.854 | 15.919 | 13.433 | 1.00 | 0.00 | −0.396 | 8.17 | −18.95 |
| ATOM | 3516 | OE2 | GLU | 213 | 18.260 | 17.924 | 14.312 | 1.00 | 0.00 | −0.427 | 8.17 | −18.95 |
| ATOM | 3518 | N | VAL | 214 | 16.196 | 15.779 | 18.349 | 1.00 | 0.00 | −0.650 | 9.00 | −17.40 |
| ATOM | 3519 | HN | VAL | 214 | 15.739 | 16.616 | 17.960 | 1.00 | 0.00 | 0.440 | 0.00 | 0.00 |
| ATOM | 3520 | CA | VAL | 214 | 16.945 | 15.858 | 19.568 | 1.00 | 0.00 | 0.158 | 9.40 | 4.00 |
| ATOM | 3521 | HA | VAL | 214 | 17.923 | 15.413 | 19.388 | 1.00 | 0.00 | 0.053 | 0.00 | 0.00 |
| ATOM | 3522 | C | VAL | 214 | 16.198 | 15.102 | 20.613 | 1.00 | 0.00 | 0.396 | 9.82 | 4.00 |
| ATOM | 3523 | O | VAL | 214 | 15.291 | 14.325 | 20.318 | 1.00 | 0.00 | −0.396 | 8.17 | −17.40 |
| ATOM | 3524 | CB | VAL | 214 | 17.147 | 17.260 | 20.069 | 1.00 | 0.00 | −0.053 | 9.40 | 4.00 |
| ATOM | 3525 | HB | VAL | 214 | 17.680 | 17.215 | 21.018 | 1.00 | 0.00 | 0.053 | 0.00 | 0.00 |
| ATOM | 3526 | CG1 | VAL | 214 | 17.970 | 18.037 | 19.028 | 1.00 | 0.00 | −0.159 | 16.15 | 4.00 |
| ATOM | 3527 | HG1 | VAL | 214 | 18.126 | 19.058 | 19.376 | 1.00 | 0.00 | 0.053 | 0.00 | 0.00 |
| ATOM | 3528 | HG1 | VAL | 214 | 18.934 | 17.549 | 18.889 | 1.00 | 0.00 | 0.053 | 0.00 | 0.00 |
| ATOM | 3529 | HG1 | VAL | 214 | 17.433 | 18.055 | 18.079 | 1.00 | 0.00 | 0.053 | 0.00 | 0.00 |
| ATOM | 3530 | CG2 | VAL | 214 | 15.775 | 17.881 | 20.374 | 1.00 | 0.00 | −0.159 | 16.15 | 4.00 |
| ATOM | 3531 | HG2 | VAL | 214 | 15.909 | 18.899 | 20.738 | 1.00 | 0.00 | 0.053 | 0.00 | 0.00 |
| ATOM | 3532 | HG2 | VAL | 214 | 15.173 | 17.897 | 19.465 | 1.00 | 0.00 | 0.053 | 0.00 | 0.00 |
| ATOM | 3533 | HG2 | VAL | 214 | 15.267 | 17.287 | 21.134 | 1.00 | 0.00 | 0.053 | 0.00 | 0.00 |
| ATOM | 3534 | N | VAL | 215 | 16.570 | 15.298 | 21.889 | 1.00 | 0.00 | −0.650 | 9.00 | −17.40 |
| ATOM | 3535 | HN | VAL | 215 | 17.329 | 15.949 | 22.131 | 1.00 | 0.00 | 0.440 | 0.00 | 0.00 |
| ATOM | 3536 | CA | VAL | 215 | 15.870 | 14.567 | 22.895 | 1.00 | 0.00 | 0.158 | 9.40 | 4.00 |
| ATOM | 3537 | HA | VAL | 215 | 15.894 | 13.514 | 22.613 | 1.00 | 0.00 | 0.053 | 0.00 | 0.00 |
| ATOM | 3538 | C | VAL | 215 | 14.472 | 15.085 | 22.936 | 1.00 | 0.00 | 0.396 | 9.82 | 4.00 |
| ATOM | 3539 | O | VAL | 215 | 14.187 | 16.130 | 23.519 | 1.00 | 0.00 | −0.396 | 8.17 | −17.40 |
| ATOM | 3540 | CB | VAL | 215 | 16.491 | 14.671 | 24.261 | 1.00 | 0.00 | −0.053 | 9.40 | 4.00 |
| ATOM | 3541 | HB | VAL | 215 | 17.461 | 14.174 | 24.266 | 1.00 | 0.00 | 0.053 | 0.00 | 0.00 |
| ATOM | 3542 | CG1 | VAL | 215 | 16.683 | 16.151 | 24.638 | 1.00 | 0.00 | −0.159 | 16.15 | 4.00 |
| ATOM | 3543 | HG1 | VAL | 215 | 17.133 | 16.218 | 25.628 | 1.00 | 0.00 | 0.053 | 0.00 | 0.00 |
| ATOM | 3544 | HG1 | VAL | 215 | 17.336 | 16.630 | 23.909 | 1.00 | 0.00 | 0.053 | 0.00 | 0.00 |
| ATOM | 3545 | HG1 | VAL | 215 | 15.715 | 16.653 | 24.644 | 1.00 | 0.00 | 0.053 | 0.00 | 0.00 |
| ATOM | 3546 | CG2 | VAL | 215 | 15.596 | 13.894 | 25.241 | 1.00 | 0.00 | −0.159 | 16.15 | 4.00 |
| ATOM | 3547 | HG2 | VAL | 215 | 16.019 | 13.950 | 26.243 | 1.00 | 0.00 | 0.053 | 0.00 | 0.00 |
| ATOM | 3548 | HG2 | VAL | 215 | 14.596 | 14.329 | 25.244 | 1.00 | 0.00 | 0.053 | 0.00 | 0.00 |
| ATOM | 3549 | HG2 | VAL | 215 | 15.536 | 12.851 | 24.930 | 1.00 | 0.00 | 0.053 | 0.00 | 0.00 |
| ATOM | 3550 | N | ASN | 216 | 13.549 | 14.360 | 22.278 | 1.00 | 0.00 | −0.650 | 9.00 | −17.40 |
| ATOM | 3551 | HN | ASN | 216 | 13.815 | 13.504 | 21.769 | 1.00 | 0.00 | 0.440 | 0.00 | 0.00 |
| ATOM | 3552 | CA | ASN | 216 | 12.192 | 14.802 | 22.300 | 1.00 | 0.00 | 0.158 | 9.40 | 4.00 |
| ATOM | 3553 | HA | ASN | 216 | 11.944 | 15.092 | 23.320 | 1.00 | 0.00 | 0.053 | 0.00 | 0.00 |
| ATOM | 3554 | C | ASN | 216 | 11.325 | 13.673 | 21.848 | 1.00 | 0.00 | 0.396 | 9.82 | 4.00 |
| ATOM | 3555 | O | ASN | 216 | 11.238 | 12.631 | 22.495 | 1.00 | 0.00 | −0.396 | 8.17 | −17.40 |
| ATOM | 3556 | CB | ASN | 216 | 11.914 | 15.989 | 21.363 | 1.00 | 0.00 | −0.106 | 12.77 | 4.00 |
| ATOM | 3557 | HB1 | ASN | 216 | 11.968 | 15.623 | 20.337 | 1.00 | 0.00 | 0.053 | 0.00 | 0.00 |
| ATOM | 3558 | HB2 | ASN | 216 | 12.675 | 16.746 | 21.550 | 1.00 | 0.00 | 0.053 | 0.00 | 0.00 |
| ATOM | 3559 | CG | ASN | 216 | 10.522 | 16.519 | 21.684 | 1.00 | 0.00 | 0.396 | 9.82 | 4.00 |
| ATOM | 3560 | OD1 | ASN | 216 | 9.779 | 15.910 | 22.452 | 1.00 | 0.00 | −0.396 | 8.17 | −17.40 |
| ATOM | 3561 | ND2 | ASN | 216 | 10.152 | 17.675 | 21.073 | 1.00 | 0.00 | −0.879 | 13.25 | −17.40 |
| ATOM | 3562 | HD2 | ASN | 216 | 10.805 | 18.153 | 20.436 | 1.00 | 0.00 | 0.440 | 0.00 | 0.00 |
| ATOM | 3563 | HD2 | ASN | 216 | 9.217 | 18.072 | 21.246 | 1.00 | 0.00 | 0.440 | 0.00 | 0.00 |
| ATOM | 3564 | N | GLY | 217 | 10.663 | 13.878 | 20.695 | 1.00 | 0.00 | −0.650 | 9.00 | −17.40 |
| ATOM | 3565 | HN | GLY | 217 | 10.851 | 14.747 | 20.176 | 1.00 | 0.00 | 0.440 | 0.00 | 0.00 |
| ATOM | 3566 | CA | GLY | 217 | 9.711 | 12.959 | 20.145 | 1.00 | 0.00 | 0.105 | 9.40 | 4.00 |
| ATOM | 3567 | HA1 | GLY | 217 | 9.241 | 13.368 | 19.250 | 1.00 | 0.00 | 0.053 | 0.00 | 0.00 |
| ATOM | 3568 | HA2 | GLY | 217 | 8.921 | 12.735 | 20.862 | 1.00 | 0.00 | 0.053 | 0.00 | 0.00 |
| ATOM | 3569 | C | GLY | 217 | 10.364 | 11.670 | 19.768 | 1.00 | 0.00 | 0.396 | 9.82 | 4.00 |
| ATOM | 3570 | O | GLY | 217 | 9.749 | 10.610 | 19.865 | 1.00 | 0.00 | −0.396 | 8.17 | −17.40 |
| ATOM | 3571 | N | LYS | 218S | 11.629 | 11.712 | 19.320 | 1.00 | 0.00 | −0.650 | 9.00 | −17.40 |
| ATOM | 3572 | HN | LYS | 218S | 12.150 | 12.600 | 19.309 | 1.00 | 0.00 | 0.440 | 0.00 | 0.00 |
| ATOM | 3573 | CA | LYS | 218S | 12.243 | 10.502 | 18.857 | 1.00 | 0.00 | 0.158 | 9.40 | 4.00 |
| ATOM | 3574 | HA | LYS | 218S | 11.668 | 10.109 | 18.018 | 1.00 | 0.00 | 0.053 | 0.00 | 0.00 |
| ATOM | 3575 | C | LYS | 218S | 12.260 | 9.507 | 19.971 | 1.00 | 0.00 | 0.396 | 9.82 | 4.00 |
| ATOM | 3576 | O | LYS | 218S | 12.019 | 8.320 | 19.755 | 1.00 | 0.00 | −0.396 | 8.17 | −17.40 |
| ATOM | 3577 | CB | LYS | 218S | 13.695 | 10.705 | 18.385 | 1.00 | 0.00 | −0.106 | 12.77 | 4.00 |
| ATOM | 3578 | HB1 | LYS | 218S | 13.702 | 11.485 | 17.623 | 1.00 | 0.00 | 0.053 | 0.00 | 0.00 |
| ATOM | 3579 | HB2 | LYS | 218S | 14.056 | 9.763 | 17.971 | 1.00 | 0.00 | 0.053 | 0.00 | 0.00 |
| ATOM | 3580 | CG | LYS | 218S | 14.661 | 11.128 | 19.495 | 1.00 | 0.00 | −0.106 | 12.77 | 4.00 |
| ATOM | 3581 | HG1 | LYS | 218S | 14.564 | 10.531 | 20.402 | 1.00 | 0.00 | 0.053 | 0.00 | 0.00 |
| ATOM | 3582 | HG2 | LYS | 218S | 14.527 | 12.161 | 19.814 | 1.00 | 0.00 | 0.053 | 0.00 | 0.00 |
| ATOM | 3583 | CD | LYS | 218S | 16.135 | 11.017 | 19.093 | 1.00 | 0.00 | −0.106 | 12.77 | 4.00 |
| ATOM | 3584 | HD1 | LYS | 218S | 16.403 | 11.630 | 18.233 | 1.00 | 0.00 | 0.053 | 0.00 | 0.00 |
| ATOM | 3585 | HD2 | LYS | 218S | 16.441 | 10.006 | 18.821 | 1.00 | 0.00 | 0.053 | 0.00 | 0.00 |
| ATOM | 3586 | CE | LYS | 218S | 17.109 | 11.440 | 20.196 | 1.00 | 0.00 | 0.099 | 12.77 | 4.00 |
| ATOM | 3587 | HE1 | LYS | 218S | 16.974 | 10.810 | 21.075 | 1.00 | 0.00 | 0.053 | 0.00 | 0.00 |
| ATOM | 3588 | HE2 | LYS | 218S | 16.929 | 12.478 | 20.475 | 1.00 | 0.00 | 0.053 | 0.00 | 0.00 |
| ATOM | 3589 | NZ | LYS | 218S | 18.504 | 11.306 | 19.720 | 1.00 | 0.00 | −0.045 | 13.25 | −39.20 |
| ATOM | 3590 | HZ1 | LYS | 218S | 19.151 | 11.592 | 20.468 | 1.00 | 0.00 | 0.280 | 0.00 | 0.00 |
| ATOM | 3591 | HZ2 | LYS | 218S | 18.687 | 10.325 | 19.464 | 1.00 | 0.00 | 0.280 | 0.00 | 0.00 |
| ATOM | 3592 | HZ3 | LYS | 218S | 18.645 | 11.907 | 18.895 | 1.00 | 0.00 | 0.280 | 0.00 | 0.00 |
| ATOM | 3593 | N | LEU | 219 | 12.531 | 9.972 | 21.202 | 1.00 | 0.00 | −0.650 | 9.00 | −17.40 |
| ATOM | 3594 | HN | LEU | 219 | 12.662 | 10.983 | 21.343 | 1.00 | 0.00 | 0.440 | 0.00 | 0.00 |
| ATOM | 3595 | CA | LEU | 219 | 12.640 | 9.085 | 22.322 | 1.00 | 0.00 | 0.158 | 9.40 | 4.00 |
| ATOM | 3596 | HA | LEU | 219 | 13.380 | 8.306 | 22.136 | 1.00 | 0.00 | 0.053 | 0.00 | 0.00 |
| ATOM | 3597 | C | LEU | 219 | 11.332 | 8.412 | 22.608 | 1.00 | 0.00 | 0.396 | 9.82 | 4.00 |
| ATOM | 3598 | O | LEU | 219 | 11.320 | 7.234 | 22.959 | 1.00 | 0.00 | −0.396 | 8.17 | −17.40 |
| ATOM | 3599 | CB | LEU | 219 | 13.095 | 9.794 | 23.610 | 1.00 | 0.00 | −0.106 | 12.77 | 4.00 |
| ATOM | 3600 | HB1 | LEU | 219 | 13.082 | 9.140 | 24.482 | 1.00 | 0.00 | 0.053 | 0.00 | 0.00 |
| ATOM | 3601 | HB2 | LEU | 219 | 12.468 | 10.646 | 23.870 | 1.00 | 0.00 | 0.053 | 0.00 | 0.00 |
| ATOM | 3602 | CG | LEU | 219 | 14.531 | 10.343 | 23.519 | 1.00 | 0.00 | −0.053 | 9.40 | 4.00 |
| ATOM | 3603 | HG | LEU | 219 | 14.625 | 11.078 | 22.720 | 1.00 | 0.00 | 0.053 | 0.00 | 0.00 |
| ATOM | 3604 | CD1 | LEU | 219 | 14.954 | 11.027 | 24.829 | 1.00 | 0.00 | −0.159 | 16.15 | 4.00 |
| ATOM | 3605 | HD1 | LEU | 219 | 15.972 | 11.403 | 24.730 | 1.00 | 0.00 | 0.053 | 0.00 | 0.00 |
| ATOM | 3606 | HD1 | LEU | 219 | 14.279 | 11.856 | 25.040 | 1.00 | 0.00 | 0.053 | 0.00 | 0.00 |
| ATOM | 3607 | HD1 | LEU | 219 | 14.910 | 10.306 | 25.646 | 1.00 | 0.00 | 0.053 | 0.00 | 0.00 |
| ATOM | 3608 | CD2 | LEU | 219 | 15.516 | 9.248 | 23.073 | 1.00 | 0.00 | −0.159 | 16.15 | 4.00 |
| ATOM | 3609 | HD2 | LEU | 219 | 16.521 | 9.664 | 23.017 | 1.00 | 0.00 | 0.053 | 0.00 | 0.00 |
| ATOM | 3610 | HD2 | LEU | 219 | 15.501 | 8.429 | 23.793 | 1.00 | 0.00 | 0.053 | 0.00 | 0.00 |
| ATOM | 3611 | HD2 | LEU | 219 | 15.223 | 8.873 | 22.092 | 1.00 | 0.00 | 0.053 | 0.00 | 0.00 |
| ATOM | 3612 | N | THR | 220 | 10.197 | 9.127 | 22.463 | 1.00 | 0.00 | −0.650 | 9.00 | −17.40 |
| ATOM | 3613 | HN | THR | 220 | 10.234 | 10.087 | 22.092 | 1.00 | 0.00 | 0.440 | 0.00 | 0.00 |
| ATOM | 3614 | CA | THR | 220 | 8.934 | 8.543 | 22.828 | 1.00 | 0.00 | 0.158 | 9.40 | 4.00 |
| ATOM | 3615 | HA | THR | 220 | 8.912 | 8.335 | 23.897 | 1.00 | 0.00 | 0.053 | 0.00 | 0.00 |
| ATOM | 3616 | C | THR | 220 | 8.725 | 7.262 | 22.083 | 1.00 | 0.00 | 0.396 | 9.82 | 4.00 |
| ATOM | 3617 | O | THR | 220 | 8.766 | 7.205 | 20.855 | 1.00 | 0.00 | −0.396 | 8.17 | −17.40 |
| ATOM | 3618 | CB | THR | 220 | 7.747 | 9.443 | 22.610 | 1.00 | 0.00 | 0.060 | 9.40 | 4.00 |
| ATOM | 3619 | HB | THR | 220 | 7.934 | 10.414 | 23.066 | 1.00 | 0.00 | 0.053 | 0.00 | 0.00 |
| ATOM | 3620 | OG1 | THR | 220 | 6.595 | 8.881 | 23.221 | 1.00 | 0.00 | −0.537 | 11.04 | −17.40 |
| ATOM | 3621 | HG1 | THR | 220 | 5.931 | 9.631 | 23.460 | 1.00 | 0.00 | 0.424 | 0.00 | 0.00 |
| ATOM | 3622 | CG2 | THR | 220 | 7.508 | 9.628 | 21.103 | 1.00 | 0.00 | −0.159 | 16.15 | 4.00 |
| ATOM | 3623 | HG2 | THR | 220 | 6.648 | 10.279 | 20.948 | 1.00 | 0.00 | 0.053 | 0.00 | 0.00 |
| ATOM | 3624 | HG2 | THR | 220 | 8.390 | 10.077 | 20.647 | 1.00 | 0.00 | 0.053 | 0.00 | 0.00 |
| ATOM | 3625 | HG2 | THR | 220 | 7.315 | 8.658 | 20.643 | 1.00 | 0.00 | 0.053 | 0.00 | 0.00 |
| ATOM | 3626 | N | ASN | 221 | 8.504 | 6.174 | 22.843 | 1.00 | 0.00 | −0.650 | 9.00 | −17.40 |
| ATOM | 3627 | HN | ASN | 221 | 8.466 | 6.265 | 23.868 | 1.00 | 0.00 | 0.440 | 0.00 | 0.00 |
| ATOM | 3628 | CA | ASN | 221 | 8.321 | 4.892 | 22.237 | 1.00 | 0.00 | 0.158 | 9.40 | 4.00 |
| ATOM | 3629 | HA | ASN | 221 | 8.959 | 4.837 | 21.355 | 1.00 | 0.00 | 0.053 | 0.00 | 0.00 |
| ATOM | 3630 | C | ASN | 221 | 6.887 | 4.757 | 21.857 | 1.00 | 0.00 | 0.396 | 9.82 | 4.00 |
| ATOM | 3631 | O | ASN | 221 | 6.007 | 5.399 | 22.430 | 1.00 | 0.00 | −0.396 | 8.17 | −17.40 |
| ATOM | 3632 | CB | ASN | 221 | 8.666 | 3.717 | 23.167 | 1.00 | 0.00 | −0.106 | 12.77 | 4.00 |
| ATOM | 3633 | HB1 | ASN | 221 | 9.699 | 3.837 | 23.491 | 1.00 | 0.00 | 0.053 | 0.00 | 0.00 |
| ATOM | 3634 | HB2 | ASN | 221 | 8.540 | 2.793 | 22.602 | 1.00 | 0.00 | 0.053 | 0.00 | 0.00 |
| ATOM | 3635 | CG | ASN | 221 | 7.714 | 3.763 | 24.354 | 1.00 | 0.00 | 0.396 | 9.82 | 4.00 |
| ATOM | 3636 | OD1 | ASN | 221 | 7.462 | 4.825 | 24.922 | 1.00 | 0.00 | −0.396 | 8.17 | −17.40 |
| ATOM | 3637 | ND2 | ASN | 221 | 7.163 | 2.581 | 24.737 | 1.00 | 0.00 | −0.879 | 13.25 | −17.40 |
| ATOM | 3638 | HD2 | ASN | 221 | 7.401 | 1.714 | 24.233 | 1.00 | 0.00 | 0.440 | 0.00 | 0.00 |
| ATOM | 3639 | HD2 | ASN | 221 | 6.507 | 2.551 | 25.530 | 1.00 | 0.00 | 0.440 | 0.00 | 0.00 |
| ATOM | 3640 | N | THR | 222 | 6.622 | 3.909 | 20.849 | 1.00 | 0.00 | −0.650 | 9.00 | −17.40 |
| ATOM | 3641 | HN | THR | 222 | 7.394 | 3.423 | 20.371 | 1.00 | 0.00 | 0.440 | 0.00 | 0.00 |
| ATOM | 3642 | CA | THR | 222 | 5.275 | 3.677 | 20.436 | 1.00 | 0.00 | 0.158 | 9.40 | 4.00 |
| ATOM | 3643 | HA | THR | 222 | 4.703 | 4.510 | 20.843 | 1.00 | 0.00 | 0.053 | 0.00 | 0.00 |
| ATOM | 3644 | C | THR | 222 | 4.906 | 2.363 | 21.028 | 1.00 | 0.00 | 0.396 | 9.82 | 4.00 |
| ATOM | 3645 | O | THR | 222 | 5.769 | 1.511 | 21.231 | 1.00 | 0.00 | −0.396 | 8.17 | −17.40 |
| ATOM | 3646 | CB | THR | 222 | 5.116 | 3.565 | 18.948 | 1.00 | 0.00 | 0.060 | 9.40 | 4.00 |
| ATOM | 3647 | HB | THR | 222 | 5.727 | 2.734 | 18.595 | 1.00 | 0.00 | 0.053 | 0.00 | 0.00 |
| ATOM | 3648 | OG1 | THR | 222 | 5.550 | 4.761 | 18.318 | 1.00 | 0.00 | −0.537 | 11.04 | −17.40 |
| ATOM | 3649 | HG1 | THR | 222 | 6.473 | 5.029 | 18.688 | 1.00 | 0.00 | 0.424 | 0.00 | 0.00 |
| ATOM | 3650 | CG2 | THR | 222 | 3.633 | 3.309 | 18.632 | 1.00 | 0.00 | −0.159 | 16.15 | 4.00 |
| ATOM | 3651 | HG2 | THR | 222 | 3.500 | 3.225 | 17.553 | 1.00 | 0.00 | 0.053 | 0.00 | 0.00 |
| ATOM | 3652 | HG2 | THR | 222 | 3.313 | 2.383 | 19.110 | 1.00 | 0.00 | 0.053 | 0.00 | 0.00 |
| ATOM | 3653 | HG2 | THR | 222 | 3.032 | 4.137 | 19.008 | 1.00 | 0.00 | 0.053 | 0.00 | 0.00 |
| ATOM | 3654 | N | GLY | 223 | 3.612 | 2.185 | 21.358 | 1.00 | 0.00 | −0.650 | 9.00 | −17.40 |
| ATOM | 3655 | HN | GLY | 223 | 2.930 | 2.941 | 21.200 | 1.00 | 0.00 | 0.440 | 0.00 | 0.00 |
| ATOM | 3656 | CA | GLY | 223 | 3.186 | 0.944 | 21.928 | 1.00 | 0.00 | 0.105 | 9.40 | 4.00 |
| ATOM | 3657 | HA1 | GLY | 223 | 2.104 | 1.019 | 22.043 | 1.00 | 0.00 | 0.053 | 0.00 | 0.00 |
| ATOM | 3658 | HA2 | GLY | 223 | 3.704 | 0.855 | 22.882 | 1.00 | 0.00 | 0.053 | 0.00 | 0.00 |
| ATOM | 3659 | C | GLY | 223 | 3.585 | −0.113 | 20.961 | 1.00 | 0.00 | 0.396 | 9.82 | 4.00 |
| ATOM | 3660 | O | GLY | 223 | 3.162 | −0.115 | 19.807 | 1.00 | 0.00 | −0.396 | 8.17 | −17.40 |
| ATOM | 3661 | N | THR | 224 | 4.416 | −1.055 | 21.435 | 1.00 | 0.00 | −0.650 | 9.00 | −17.40 |
| ATOM | 3662 | HN | THR | 224 | 4.669 | −1.058 | 22.433 | 1.00 | 0.00 | 0.440 | 0.00 | 0.00 |
| ATOM | 3663 | CA | THR | 224 | 4.950 | −2.052 | 20.565 | 1.00 | 0.00 | 0.158 | 9.40 | 4.00 |
| ATOM | 3664 | HA | THR | 224 | 5.015 | −1.659 | 19.550 | 1.00 | 0.00 | 0.053 | 0.00 | 0.00 |
| ATOM | 3665 | C | THR | 224 | 4.067 | −3.249 | 20.569 | 1.00 | 0.00 | 0.396 | 9.82 | 4.00 |
| ATOM | 3666 | O | THR | 224 | 2.843 | −3.149 | 20.591 | 1.00 | 0.00 | −0.396 | 8.17 | −17.40 |
| ATOM | 3667 | CB | THR | 224 | 6.319 | −2.505 | 20.977 | 1.00 | 0.00 | 0.060 | 9.40 | 4.00 |
| ATOM | 3668 | HB | THR | 224 | 6.667 | −3.253 | 20.264 | 1.00 | 0.00 | 0.053 | 0.00 | 0.00 |
| ATOM | 3669 | OG1 | THR | 224 | 6.276 | −3.087 | 22.272 | 1.00 | 0.00 | −0.537 | 11.04 | −17.40 |
| ATOM | 3670 | HG1 | THR | 224 | 5.867 | −2.415 | 22.937 | 1.00 | 0.00 | 0.424 | 0.00 | 0.00 |
| ATOM | 3671 | CG2 | THR | 224 | 7.260 | −1.289 | 20.976 | 1.00 | 0.00 | −0.159 | 16.15 | 4.00 |
| ATOM | 3672 | HG2 | THR | 224 | 8.260 | −1.602 | 21.273 | 1.00 | 0.00 | 0.053 | 0.00 | 0.00 |
| ATOM | 3673 | HG2 | THR | 224 | 7.297 | −0.858 | 19.975 | 1.00 | 0.00 | 0.053 | 0.00 | 0.00 |
| ATOM | 3674 | HG2 | THR | 224 | 6.890 | −0.542 | 21.678 | 1.00 | 0.00 | 0.053 | 0.00 | 0.00 |
| ATOM | 3675 | N | VAL | 225 | 4.709 | −4.429 | 20.547 | 1.00 | 0.00 | −0.650 | 9.00 | −17.40 |
| ATOM | 3676 | HN | VAL | 225 | 5.738 | −4.426 | 20.578 | 1.00 | 0.00 | 0.440 | 0.00 | 0.00 |
| ATOM | 3677 | CA | VAL | 225 | 4.035 | −5.688 | 20.482 | 1.00 | 0.00 | 0.158 | 9.40 | 4.00 |
| ATOM | 3678 | HA | VAL | 225 | 3.437 | −5.717 | 19.571 | 1.00 | 0.00 | 0.053 | 0.00 | 0.00 |
| ATOM | 3679 | C | VAL | 225 | 3.158 | −5.822 | 21.679 | 1.00 | 0.00 | 0.396 | 9.82 | 4.00 |
| ATOM | 3680 | O | VAL | 225 | 2.124 | −6.485 | 21.623 | 1.00 | 0.00 | −0.396 | 8.17 | −17.40 |
| ATOM | 3681 | CB | VAL | 225 | 4.985 | −6.848 | 20.498 | 1.00 | 0.00 | −0.053 | 9.40 | 4.00 |
| ATOM | 3682 | HB | VAL | 225 | 4.403 | −7.770 | 20.493 | 1.00 | 0.00 | 0.053 | 0.00 | 0.00 |
| ATOM | 3683 | CG1 | VAL | 225 | 5.874 | −6.766 | 19.246 | 1.00 | 0.00 | −0.159 | 16.15 | 4.00 |
| ATOM | 3684 | HG1 | VAL | 225 | 6.572 | −7.602 | 19.240 | 1.00 | 0.00 | 0.053 | 0.00 | 0.00 |
| ATOM | 3685 | HG1 | VAL | 225 | 5.250 | −6.808 | 18.353 | 1.00 | 0.00 | 0.053 | 0.00 | 0.00 |
| ATOM | 3686 | HG1 | VAL | 225 | 6.430 | −5.828 | 19.255 | 1.00 | 0.00 | 0.053 | 0.00 | 0.00 |
| ATOM | 3687 | CG2 | VAL | 225 | 5.773 | −6.818 | 21.820 | 1.00 | 0.00 | −0.159 | 16.15 | 4.00 |
| ATOM | 3688 | HG2 | VAL | 225 | 6.469 | −7.656 | 21.847 | 1.00 | 0.00 | 0.053 | 0.00 | 0.00 |
| ATOM | 3689 | HG2 | VAL | 225 | 6.328 | −5.882 | 21.891 | 1.00 | 0.00 | 0.053 | 0.00 | 0.00 |
| ATOM | 3690 | HG2 | VAL | 225 | 5.080 | −6.893 | 22.658 | 1.00 | 0.00 | 0.053 | 0.00 | 0.00 |
| ATOM | 3691 | N | LYS | 226S | 3.541 | −5.188 | 22.800 | 1.00 | 0.00 | −0.650 | 9.00 | −17.40 |
| ATOM | 3692 | HN | LYS | 226S | 4.377 | −4.587 | 22.807 | 1.00 | 0.00 | 0.440 | 0.00 | 0.00 |
| ATOM | 3693 | CA | LYS | 226S | 2.757 | −5.369 | 23.982 | 1.00 | 0.00 | 0.158 | 9.40 | 4.00 |
| ATOM | 3694 | HA | LYS | 226S | 2.607 | −6.434 | 24.153 | 1.00 | 0.00 | 0.053 | 0.00 | 0.00 |
| ATOM | 3695 | C | LYS | 226S | 1.440 | −4.699 | 23.804 | 1.00 | 0.00 | 0.396 | 9.82 | 4.00 |
| ATOM | 3696 | O | LYS | 226S | 1.353 | −3.478 | 23.675 | 1.00 | 0.00 | −0.396 | 8.17 | −17.40 |
| ATOM | 3697 | CB | LYS | 226S | 3.411 | −4.799 | 25.252 | 1.00 | 0.00 | −0.106 | 12.77 | 4.00 |
| ATOM | 3698 | HB1 | LYS | 226S | 2.754 | −4.826 | 26.121 | 1.00 | 0.00 | 0.053 | 0.00 | 0.00 |
| ATOM | 3699 | HB2 | LYS | 226S | 3.714 | −3.757 | 25.147 | 1.00 | 0.00 | 0.053 | 0.00 | 0.00 |
| ATOM | 3700 | CG | LYS | 226S | 4.674 | −5.553 | 25.669 | 1.00 | 0.00 | −0.106 | 12.77 | 4.00 |
| ATOM | 3701 | HG1 | LYS | 226S | 5.138 | −5.151 | 26.569 | 1.00 | 0.00 | 0.053 | 0.00 | 0.00 |
| ATOM | 3702 | HG2 | LYS | 226S | 5.455 | −5.538 | 24.909 | 1.00 | 0.00 | 0.053 | 0.00 | 0.00 |
| ATOM | 3703 | CD | LYS | 226S | 4.425 | −7.034 | 25.967 | 1.00 | 0.00 | −0.106 | 12.77 | 4.00 |
| ATOM | 3704 | HD1 | LYS | 226S | 5.337 | −7.622 | 26.063 | 1.00 | 0.00 | 0.053 | 0.00 | 0.00 |
| ATOM | 3705 | HD2 | LYS | 226S | 3.842 | −7.545 | 25.200 | 1.00 | 0.00 | 0.053 | 0.00 | 0.00 |
| ATOM | 3706 | CE | LYS | 226S | 3.660 | −7.282 | 27.270 | 1.00 | 0.00 | 0.099 | 12.77 | 4.00 |
| ATOM | 3707 | HE1 | LYS | 226S | 2.677 | −6.811 | 27.223 | 1.00 | 0.00 | 0.053 | 0.00 | 0.00 |
| ATOM | 3708 | HE2 | LYS | 226S | 4.210 | −6.863 | 28.112 | 1.00 | 0.00 | 0.053 | 0.00 | 0.00 |
| ATOM | 3709 | NZ | LYS | 226S | 3.484 | −8.736 | 27.488 | 1.00 | 0.00 | −0.045 | 13.25 | −39.20 |
| ATOM | 3710 | HZ1 | LYS | 226S | 2.968 | −8.892 | 28.366 | 1.00 | 0.00 | 0.280 | 0.00 | 0.00 |
| ATOM | 3711 | HZ2 | LYS | 226S | 2.953 | −9.139 | 26.702 | 1.00 | 0.00 | 0.280 | 0.00 | 0.00 |
| ATOM | 3712 | HZ3 | LYS | 226S | 4.407 | −9.189 | 27.546 | 1.00 | 0.00 | 0.280 | 0.00 | 0.00 |
| ATOM | 3713 | N | MET | 227 | 0.368 | −5.512 | 23.778 | 1.00 | 0.00 | −0.650 | 9.00 | −17.40 |
| ATOM | 3714 | HN | MET | 227 | 0.497 | −6.532 | 23.827 | 1.00 | 0.00 | 0.440 | 0.00 | 0.00 |
| ATOM | 3715 | CA | MET | 227 | −0.948 | −4.967 | 23.682 | 1.00 | 0.00 | 0.158 | 9.40 | 4.00 |
| ATOM | 3716 | HA | MET | 227 | −0.860 | −4.143 | 22.973 | 1.00 | 0.00 | 0.053 | 0.00 | 0.00 |
| ATOM | 3717 | C | MET | 227 | −1.284 | −4.530 | 25.064 | 1.00 | 0.00 | 0.396 | 9.82 | 4.00 |
| ATOM | 3718 | O | MET | 227 | −0.651 | −4.952 | 26.031 | 1.00 | 0.00 | −0.396 | 8.17 | −17.40 |
| ATOM | 3719 | CB | MET | 227 | −2.020 | −5.982 | 23.248 | 1.00 | 0.00 | −0.106 | 12.77 | 4.00 |
| ATOM | 3720 | HB1 | MET | 227 | −3.028 | −5.569 | 23.220 | 1.00 | 0.00 | 0.053 | 0.00 | 0.00 |
| ATOM | 3721 | HB2 | MET | 227 | −2.090 | −6.851 | 23.901 | 1.00 | 0.00 | 0.053 | 0.00 | 0.00 |
| ATOM | 3722 | CG | MET | 227 | −1.792 | −6.558 | 21.850 | 1.00 | 0.00 | −0.041 | 12.77 | 4.00 |
| ATOM | 3723 | HG1 | MET | 227 | −1.551 | −5.735 | 21.176 | 1.00 | 0.00 | 0.053 | 0.00 | 0.00 |
| ATOM | 3724 | HG2 | MET | 227 | −2.706 | −7.058 | 21.532 | 1.00 | 0.00 | 0.053 | 0.00 | 0.00 |
| ATOM | 3725 | SD | MET | 227 | −0.438 | −7.768 | 21.750 | 1.00 | 0.00 | −0.130 | 16.39 | −6.40 |
| ATOM | 3726 | CE | MET | 227 | −0.533 | −7.972 | 19.948 | 1.00 | 0.00 | −0.094 | 16.15 | 4.00 |
| ATOM | 3727 | HE1 | MET | 227 | 0.221 | −8.688 | 19.622 | 1.00 | 0.00 | 0.053 | 0.00 | 0.00 |
| ATOM | 3728 | HE2 | MET | 227 | −1.522 | −8.337 | 19.674 | 1.00 | 0.00 | 0.053 | 0.00 | 0.00 |
| ATOM | 3729 | HE3 | MET | 227 | −0.354 | −7.012 | 19.463 | 1.00 | 0.00 | 0.053 | 0.00 | 0.00 |
| ATOM | 3730 | N | LEU | 228 | −2.286 | −3.648 | 25.195 | 1.00 | 0.00 | −0.650 | 9.00 | −17.40 |
| ATOM | 3731 | HN | LEU | 228 | −2.799 | −3.316 | 24.366 | 1.00 | 0.00 | 0.440 | 0.00 | 0.00 |
| ATOM | 3732 | CA | LEU | 228 | −2.633 | −3.174 | 26.499 | 1.00 | 0.00 | 0.158 | 9.40 | 4.00 |
| ATOM | 3733 | HA | LEU | 228 | −1.881 | −3.571 | 27.181 | 1.00 | 0.00 | 0.053 | 0.00 | 0.00 |
| ATOM | 3734 | C | LEU | 228 | −3.995 | −3.699 | 26.790 | 1.00 | 0.00 | 0.396 | 9.82 | 4.00 |
| ATOM | 3735 | O | LEU | 228 | −4.903 | −3.610 | 25.964 | 1.00 | 0.00 | −0.396 | 8.17 | −17.40 |
| ATOM | 3736 | CB | LEU | 228 | −2.769 | −1.643 | 26.568 | 1.00 | 0.00 | −0.106 | 12.77 | 4.00 |
| ATOM | 3737 | HB1 | LEU | 228 | −2.998 | −1.368 | 27.597 | 1.00 | 0.00 | 0.053 | 0.00 | 0.00 |
| ATOM | 3738 | HB2 | LEU | 228 | −3.575 | −1.342 | 25.899 | 1.00 | 0.00 | 0.053 | 0.00 | 0.00 |
| ATOM | 3739 | CG | LEU | 228 | −1.508 | −0.863 | 26.151 | 1.00 | 0.00 | −0.053 | 9.40 | 4.00 |
| ATOM | 3740 | HG | LEU | 228 | −1.684 | 0.212 | 26.149 | 1.00 | 0.00 | 0.053 | 0.00 | 0.00 |
| ATOM | 3741 | CD1 | LEU | 228 | −0.338 | −1.131 | 27.105 | 1.00 | 0.00 | −0.159 | 16.15 | 4.00 |
| ATOM | 3742 | HD1 | LEU | 228 | 0.534 | −0.564 | 26.779 | 1.00 | 0.00 | 0.053 | 0.00 | 0.00 |
| ATOM | 3743 | HD1 | LEU | 228 | −0.613 | −0.824 | 28.114 | 1.00 | 0.00 | 0.053 | 0.00 | 0.00 |
| ATOM | 3744 | HD1 | LEU | 228 | −0.101 | −2.195 | 27.100 | 1.00 | 0.00 | 0.053 | 0.00 | 0.00 |
| ATOM | 3745 | CD2 | LEU | 228 | −1.155 | −1.110 | 24.676 | 1.00 | 0.00 | −0.159 | 16.15 | 4.00 |
| ATOM | 3746 | HD2 | LEU | 228 | −0.260 | −0.544 | 24.416 | 1.00 | 0.00 | 0.053 | 0.00 | 0.00 |
| ATOM | 3747 | HD2 | LEU | 228 | −0.970 | −2.172 | 24.519 | 1.00 | 0.00 | 0.053 | 0.00 | 0.00 |
| ATOM | 3748 | HD2 | LEU | 228 | −1.983 | −0.788 | 24.044 | 1.00 | 0.00 | 0.053 | 0.00 | 0.00 |
| ATOM | 3749 | N | PRO | 229 | −4.138 | −4.268 | 27.948 | 1.00 | 0.00 | −0.422 | 9.00 | −17.40 |
| ATOM | 3750 | CA | PRO | 229 | −5.424 | −4.778 | 28.318 | 1.00 | 0.00 | 0.158 | 9.40 | 4.00 |
| ATOM | 3751 | HA | PRO | 229 | −5.940 | −5.243 | 27.478 | 1.00 | 0.00 | 0.053 | 0.00 | 0.00 |
| ATOM | 3752 | CD | PRO | 229 | −3.062 | −5.058 | 28.517 | 1.00 | 0.00 | 0.105 | 12.77 | 4.00 |
| ATOM | 3753 | HD1 | PRO | 229 | −2.295 | −4.325 | 28.770 | 1.00 | 0.00 | 0.053 | 0.00 | 0.00 |
| ATOM | 3754 | HD2 | PRO | 229 | −2.777 | −5.741 | 27.716 | 1.00 | 0.00 | 0.053 | 0.00 | 0.00 |
| ATOM | 3755 | C | PRO | 229 | −6.329 | −3.709 | 28.834 | 1.00 | 0.00 | 0.396 | 9.82 | 4.00 |
| ATOM | 3756 | O | PRO | 229 | −5.894 | −2.785 | 29.520 | 1.00 | 0.00 | −0.396 | 8.17 | −17.40 |
| ATOM | 3757 | CB | PRO | 229 | −5.173 | −5.907 | 29.322 | 1.00 | 0.00 | −0.106 | 12.77 | 4.00 |
| ATOM | 3758 | HB1 | PRO | 229 | −5.376 | −6.822 | 28.766 | 1.00 | 0.00 | 0.053 | 0.00 | 0.00 |
| ATOM | 3759 | HB2 | PRO | 229 | −5.877 | −5.719 | 30.132 | 1.00 | 0.00 | 0.053 | 0.00 | 0.00 |
| ATOM | 3760 | CG | PRO | 229 | −3.699 | −5.746 | 29.732 | 1.00 | 0.00 | −0.106 | 12.77 | 4.00 |
| ATOM | 3761 | HG1 | PRO | 229 | −3.241 | −6.714 | 29.933 | 1.00 | 0.00 | 0.053 | 0.00 | 0.00 |
| ATOM | 3762 | HG2 | PRO | 229 | −3.608 | −5.139 | 30.632 | 1.00 | 0.00 | 0.053 | 0.00 | 0.00 |
| ATOM | 3763 | N | PRO | 230 | −7.572 | −3.900 | 28.515 | 1.00 | 0.00 | −0.422 | 9.00 | −17.40 |
| ATOM | 3764 | CA | PRO | 230 | −8.666 | −3.039 | 28.870 | 1.00 | 0.00 | 0.158 | 9.40 | 4.00 |
| ATOM | 3765 | HA | PRO | 230 | −8.543 | −2.021 | 28.498 | 1.00 | 0.00 | 0.053 | 0.00 | 0.00 |
| ATOM | 3766 | CD | PRO | 230 | −8.007 | −5.194 | 28.015 | 1.00 | 0.00 | 0.105 | 12.77 | 4.00 |
| ATOM | 3767 | HD1 | PRO | 230 | −7.468 | −5.925 | 28.617 | 1.00 | 0.00 | 0.053 | 0.00 | 0.00 |
| ATOM | 3768 | HD2 | PRO | 230 | −7.720 | −5.201 | 26.963 | 1.00 | 0.00 | 0.053 | 0.00 | 0.00 |
| ATOM | 3769 | C | PRO | 230 | −8.854 | −2.912 | 30.360 | 1.00 | 0.00 | 0.396 | 9.82 | 4.00 |
| ATOM | 3770 | O | PRO | 230 | −9.458 | −1.921 | 30.768 | 1.00 | 0.00 | −0.396 | 8.17 | −17.40 |
| ATOM | 3771 | CB | PRO | 230 | −9.902 | −3.724 | 28.283 | 1.00 | 0.00 | −0.106 | 12.77 | 4.00 |
| ATOM | 3772 | HB1 | PRO | 230 | −10.023 | −3.264 | 27.302 | 1.00 | 0.00 | 0.053 | 0.00 | 0.00 |
| ATOM | 3773 | HB2 | PRO | 230 | −10.699 | −3.488 | 28.987 | 1.00 | 0.00 | 0.053 | 0.00 | 0.00 |
| ATOM | 3774 | CG | PRO | 230 | −9.523 | −5.216 | 28.236 | 1.00 | 0.00 | −0.106 | 12.77 | 4.00 |
| ATOM | 3775 | HG1 | PRO | 230 | −10.041 | −5.725 | 27.423 | 1.00 | 0.00 | 0.053 | 0.00 | 0.00 |
| ATOM | 3776 | HG2 | PRO | 230 | −9.787 | −5.715 | 29.167 | 1.00 | 0.00 | 0.053 | 0.00 | 0.00 |
| ATOM | 3777 | N | LEU | 231 | −8.376 | −3.896 | 31.160 | 1.00 | 0.00 | −0.650 | 9.00 | −17.40 |
| ATOM | 3778 | HN | LEU | 231 | −7.784 | −4.610 | 30.712 | 1.00 | 0.00 | 0.440 | 0.00 | 0.00 |
| ATOM | 3779 | CA | LEU | 231 | −8.612 | −4.044 | 32.579 | 1.00 | 0.00 | 0.158 | 9.40 | 4.00 |
| ATOM | 3780 | HA | LEU | 231 | −9.600 | −4.482 | 32.715 | 1.00 | 0.00 | 0.053 | 0.00 | 0.00 |
| ATOM | 3781 | C | LEU | 231 | −8.575 | −2.763 | 33.361 | 1.00 | 0.00 | 0.396 | 9.82 | 4.00 |
| ATOM | 3782 | O | LEU | 231 | −8.118 | −1.718 | 32.904 | 1.00 | 0.00 | −0.396 | 8.17 | −17.40 |
| ATOM | 3783 | CB | LEU | 231 | −7.692 | −5.076 | 33.257 | 1.00 | 0.00 | −0.106 | 12.77 | 4.00 |
| ATOM | 3784 | HB1 | LEU | 231 | −7.965 | −6.066 | 32.893 | 1.00 | 0.00 | 0.053 | 0.00 | 0.00 |
| ATOM | 3785 | HB2 | LEU | 231 | −7.838 | −5.006 | 34.334 | 1.00 | 0.00 | 0.053 | 0.00 | 0.00 |
| ATOM | 3786 | CG | LEU | 231 | −6.192 | −4.879 | 32.980 | 1.00 | 0.00 | −0.053 | 9.40 | 4.00 |
| ATOM | 3787 | HG | LEU | 231 | −5.974 | −4.860 | 31.912 | 1.00 | 0.00 | 0.053 | 0.00 | 0.00 |
| ATOM | 3788 | CD1 | LEU | 231 | −5.670 | −3.559 | 33.566 | 1.00 | 0.00 | −0.159 | 16.15 | 4.00 |
| ATOM | 3789 | HD1 | LEU | 231 | −4.606 | −3.459 | 33.347 | 1.00 | 0.00 | 0.053 | 0.00 | 0.00 |
| ATOM | 3790 | HD1 | LEU | 231 | −6.211 | −2.724 | 33.121 | 1.00 | 0.00 | 0.053 | 0.00 | 0.00 |
| ATOM | 3791 | HD1 | LEU | 231 | −5.820 | −3.555 | 34.645 | 1.00 | 0.00 | 0.053 | 0.00 | 0.00 |
| ATOM | 3792 | CD2 | LEU | 231 | −5.389 | −6.103 | 33.448 | 1.00 | 0.00 | −0.159 | 16.15 | 4.00 |
| ATOM | 3793 | HD2 | LEU | 231 | −4.330 | −5.943 | 33.243 | 1.00 | 0.00 | 0.053 | 0.00 | 0.00 |
| ATOM | 3794 | HD2 | LEU | 231 | −5.533 | −6.245 | 34.518 | 1.00 | 0.00 | 0.053 | 0.00 | 0.00 |
| ATOM | 3795 | HD2 | LEU | 231 | −5.732 | −6.989 | 32.914 | 1.00 | 0.00 | 0.053 | 0.00 | 0.00 |
| ATOM | 3796 | N | GLU | 232 | −9.075 | −2.866 | 34.613 | 1.00 | 0.00 | −0.650 | 9.00 | −17.40 |
| ATOM | 3797 | HN | GLU | 232 | −9.351 | −3.809 | 34.920 | 1.00 | 0.00 | 0.440 | 0.00 | 0.00 |
| ATOM | 3798 | CA | GLU | 232 | −9.263 | −1.803 | 35.563 | 1.00 | 0.00 | 0.158 | 9.40 | 4.00 |
| ATOM | 3799 | HA | GLU | 232 | −10.160 | −1.231 | 35.325 | 1.00 | 0.00 | 0.053 | 0.00 | 0.00 |
| ATOM | 3800 | C | GLU | 232 | −8.097 | −0.875 | 35.547 | 1.00 | 0.00 | 0.396 | 9.82 | 4.00 |
| ATOM | 3801 | O | GLU | 232 | −6.978 | −1.260 | 35.213 | 1.00 | 0.00 | −0.396 | 8.17 | −17.40 |
| ATOM | 3802 | CB | GLU | 232 | −9.424 | −2.316 | 37.005 | 1.00 | 0.00 | −0.106 | 12.77 | 4.00 |
| ATOM | 3803 | HB1 | GLU | 232 | −9.516 | −1.517 | 37.740 | 1.00 | 0.00 | 0.053 | 0.00 | 0.00 |
| ATOM | 3804 | HB2 | GLU | 232 | −8.584 | −2.923 | 37.344 | 1.00 | 0.00 | 0.053 | 0.00 | 0.00 |
| ATOM | 3805 | CG | GLU | 232 | −10.662 | −3.192 | 37.203 | 1.00 | 0.00 | −0.106 | 12.77 | 4.00 |
| ATOM | 3806 | HG1 | GLU | 232 | −10.609 | −4.021 | 36.497 | 1.00 | 0.00 | 0.053 | 0.00 | 0.00 |
| ATOM | 3807 | HG2 | GLU | 232 | −11.545 | −2.581 | 37.014 | 1.00 | 0.00 | 0.053 | 0.00 | 0.00 |
| ATOM | 3808 | CD | GLU | 232 | −10.658 | −3.704 | 38.635 | 1.00 | 0.00 | 0.399 | 9.82 | 4.00 |
| ATOM | 3809 | OE1 | GLU | 232 | −9.748 | −3.299 | 39.407 | 1.00 | 0.00 | −0.396 | 8.17 | −18.95 |
| ATOM | 3810 | OE2 | GLU | 232 | −11.564 | −4.510 | 38.976 | 1.00 | 0.00 | −0.427 | 8.17 | −18.95 |
| ATOM | 3812 | N | THR | 233 | −8.356 | 0.404 | 35.899 | 1.00 | 0.00 | −0.650 | 9.00 | −17.40 |
| ATOM | 3813 | HN | THR | 233 | −9.317 | 0.685 | 36.137 | 1.00 | 0.00 | 0.440 | 0.00 | 0.00 |
| ATOM | 3814 | CA | THR | 233 | −7.301 | 1.369 | 35.942 | 1.00 | 0.00 | 0.158 | 9.40 | 4.00 |
| ATOM | 3815 | HA | THR | 233 | −6.350 | 0.839 | 36.012 | 1.00 | 0.00 | 0.053 | 0.00 | 0.00 |
| ATOM | 3816 | C | THR | 233 | −7.503 | 2.238 | 37.139 | 1.00 | 0.00 | 0.396 | 9.82 | 4.00 |
| ATOM | 3817 | O | THR | 233 | −8.621 | 2.598 | 37.506 | 1.00 | 0.00 | −0.396 | 8.17 | −17.40 |
| ATOM | 3818 | CB | THR | 233 | −7.262 | 2.271 | 34.745 | 1.00 | 0.00 | 0.060 | 9.40 | 4.00 |
| ATOM | 3819 | HB | THR | 233 | −8.211 | 2.802 | 34.682 | 1.00 | 0.00 | 0.053 | 0.00 | 0.00 |
| ATOM | 3820 | OG1 | THR | 233 | −7.074 | 1.506 | 33.564 | 1.00 | 0.00 | −0.537 | 11.04 | −17.40 |
| ATOM | 3821 | HG1 | THR | 233 | −7.551 | 1.966 | 32.776 | 1.00 | 0.00 | 0.424 | 0.00 | 0.00 |
| ATOM | 3822 | CG2 | THR | 233 | −6.100 | 3.264 | 34.921 | 1.00 | 0.00 | −0.159 | 16.15 | 4.00 |
| ATOM | 3823 | HG2 | THR | 233 | −6.056 | 3.929 | 34.058 | 1.00 | 0.00 | 0.053 | 0.00 | 0.00 |
| ATOM | 3824 | HG2 | THR | 233 | −6.257 | 3.852 | 35.825 | 1.00 | 0.00 | 0.053 | 0.00 | 0.00 |
| ATOM | 3825 | HG2 | THR | 233 | −5.161 | 2.715 | 35.004 | 1.00 | 0.00 | 0.053 | 0.00 | 0.00 |
| ATOM | 3826 | N | PRO | 234 | −6.409 | 2.537 | 37.776 | 1.00 | 0.00 | −0.422 | 9.00 | −17.40 |
| ATOM | 3827 | CA | PRO | 234 | −6.448 | 3.423 | 38.903 | 1.00 | 0.00 | 0.158 | 9.40 | 4.00 |
| ATOM | 3828 | HA | PRO | 234 | −7.252 | 3.116 | 39.571 | 1.00 | 0.00 | 0.053 | 0.00 | 0.00 |
| ATOM | 3829 | CD | PRO | 234 | −5.344 | 1.553 | 37.894 | 1.00 | 0.00 | 0.105 | 12.77 | 4.00 |
| ATOM | 3830 | HD1 | PRO | 234 | −4.666 | 1.756 | 37.064 | 1.00 | 0.00 | 0.053 | 0.00 | 0.00 |
| ATOM | 3831 | HD2 | PRO | 234 | −5.834 | 0.582 | 37.818 | 1.00 | 0.00 | 0.053 | 0.00 | 0.00 |
| ATOM | 3832 | C | PRO | 234 | −6.693 | 4.815 | 38.423 | 1.00 | 0.00 | 0.396 | 9.82 | 4.00 |
| ATOM | 3833 | O | PRO | 234 | −7.080 | 5.662 | 39.226 | 1.00 | 0.00 | −0.396 | 8.17 | −17.40 |
| ATOM | 3834 | CB | PRO | 234 | −5.114 | 3.242 | 39.623 | 1.00 | 0.00 | −0.106 | 12.77 | 4.00 |
| ATOM | 3835 | HB1 | PRO | 234 | −5.229 | 3.376 | 40.698 | 1.00 | 0.00 | 0.053 | 0.00 | 0.00 |
| ATOM | 3836 | HB2 | PRO | 234 | −4.379 | 3.966 | 39.271 | 1.00 | 0.00 | 0.053 | 0.00 | 0.00 |
| ATOM | 3837 | CG | PRO | 234 | −4.706 | 1.801 | 39.270 | 1.00 | 0.00 | −0.106 | 12.77 | 4.00 |
| ATOM | 3838 | HG1 | PRO | 234 | −5.129 | 1.197 | 40.072 | 1.00 | 0.00 | 0.053 | 0.00 | 0.00 |
| ATOM | 3839 | HG2 | PRO | 234 | −3.616 | 1.825 | 39.258 | 1.00 | 0.00 | 0.053 | 0.00 | 0.00 |
| ATOM | 3840 | N | LEU | 235 | −6.460 | 5.079 | 37.122 | 1.00 | 0.00 | −0.650 | 9.00 | −17.40 |
| ATOM | 3841 | HN | LEU | 235 | −6.127 | 4.333 | 36.494 | 1.00 | 0.00 | 0.440 | 0.00 | 0.00 |
| ATOM | 3842 | CA | LEU | 235 | −6.676 | 6.400 | 36.617 | 1.00 | 0.00 | 0.158 | 9.40 | 4.00 |
| ATOM | 3843 | HA | LEU | 235 | −6.074 | 7.076 | 37.224 | 1.00 | 0.00 | 0.053 | 0.00 | 0.00 |
| ATOM | 3844 | C | LEU | 235 | −8.122 | 6.683 | 36.749 | 1.00 | 0.00 | 0.396 | 9.82 | 4.00 |
| ATOM | 3845 | O | LEU | 235 | −8.504 | 7.812 | 37.049 | 1.00 | 0.00 | −0.396 | 8.17 | −17.40 |
| ATOM | 3846 | CB | LEU | 235 | −6.300 | 6.593 | 35.138 | 1.00 | 0.00 | −0.106 | 12.77 | 4.00 |
| ATOM | 3847 | HB1 | LEU | 235 | −6.657 | 7.534 | 34.721 | 1.00 | 0.00 | 0.053 | 0.00 | 0.00 |
| ATOM | 3848 | HB2 | LEU | 235 | −6.701 | 5.817 | 34.486 | 1.00 | 0.00 | 0.053 | 0.00 | 0.00 |
| ATOM | 3849 | CG | LEU | 235 | −4.783 | 6.588 | 34.891 | 1.00 | 0.00 | −0.053 | 9.40 | 4.00 |
| ATOM | 3850 | HG | LEU | 235 | −4.356 | 5.600 | 35.066 | 1.00 | 0.00 | 0.053 | 0.00 | 0.00 |
| ATOM | 3851 | CD1 | LEU | 235 | −4.451 | 6.987 | 33.444 | 1.00 | 0.00 | −0.159 | 16.15 | 4.00 |
| ATOM | 3852 | HD1 | LEU | 235 | −3.370 | 6.973 | 33.301 | 1.00 | 0.00 | 0.053 | 0.00 | 0.00 |
| ATOM | 3853 | HD1 | LEU | 235 | −4.916 | 6.281 | 32.756 | 1.00 | 0.00 | 0.053 | 0.00 | 0.00 |
| ATOM | 3854 | HD1 | LEU | 235 | −4.830 | 7.989 | 33.246 | 1.00 | 0.00 | 0.053 | 0.00 | 0.00 |
| ATOM | 3855 | CD2 | LEU | 235 | −4.057 | 7.454 | 35.934 | 1.00 | 0.00 | −0.159 | 16.15 | 4.00 |
| ATOM | 3856 | HD2 | LEU | 235 | −2.984 | 7.435 | 35.739 | 1.00 | 0.00 | 0.053 | 0.00 | 0.00 |
| ATOM | 3657 | HD2 | LEU | 235 | −4.419 | 8.480 | 35.870 | 1.00 | 0.00 | 0.053 | 0.00 | 0.00 |
| ATOM | 3858 | HD2 | LEU | 235 | −4.251 | 7.061 | 36.932 | 1.00 | 0.00 | 0.053 | 0.00 | 0.00 |
| ATOM | 3859 | N | PHE | 236 | −8.948 | 5.646 | 36.506 | 1.00 | 0.00 | −0.650 | 9.00 | −17.40 |
| ATOM | 3860 | HN | PHE | 236 | −8.540 | 4.746 | 36.212 | 1.00 | 0.00 | 0.440 | 0.00 | 0.00 |
| ATOM | 3861 | CA | PHE | 236 | −10.369 | 5.745 | 36.640 | 1.00 | 0.00 | 0.158 | 9.40 | 4.00 |
| ATOM | 3862 | HA | PHE | 236 | −10.668 | 6.606 | 36.042 | 1.00 | 0.00 | 0.053 | 0.00 | 0.00 |
| ATOM | 3863 | C | PHE | 236 | −10.632 | 5.934 | 38.092 | 1.00 | 0.00 | 0.396 | 9.82 | 4.00 |
| ATOM | 3864 | O | PHE | 236 | −10.900 | 4.976 | 38.814 | 1.00 | 0.00 | −0.396 | 8.17 | −17.40 |
| ATOM | 3865 | CB | PHE | 236 | −11.094 | 4.464 | 36.187 | 1.00 | 0.00 | −0.106 | 12.77 | 4.00 |
| ATOM | 3866 | HB1 | PHE | 236 | −10.675 | 3.613 | 36.725 | 1.00 | 0.00 | 0.053 | 0.00 | 0.00 |
| ATOM | 3867 | HB2 | PHE | 236 | −10.947 | 4.337 | 35.114 | 1.00 | 0.00 | 0.053 | 0.00 | 0.00 |
| ATOM | 3868 | CG | PHE | 236 | −12.546 | 4.599 | 36.493 | 1.00 | 0.00 | 0.000 | 7.26 | 0.60 |
| ATOM | 3869 | CD1 | PHE | 236 | −13.358 | 5.383 | 35.709 | 1.00 | 0.00 | −0.127 | 10.80 | 0.60 |
| ATOM | 3870 | HD1 | PHE | 236 | −12.937 | 5.916 | 34.856 | 1.00 | 0.00 | 0.127 | 0.00 | 0.00 |
| ATOM | 3871 | CD2 | PHE | 236 | −13.099 | 3.924 | 37.559 | 1.00 | 0.00 | −0.127 | 10.80 | 0.60 |
| ATOM | 3872 | HD2 | PHE | 236 | −12.465 | 3.292 | 38.181 | 1.00 | 0.00 | 0.127 | 0.00 | 0.00 |
| ATOM | 3873 | CE1 | PHE | 236 | −14.697 | 5.497 | 35.995 | 1.00 | 0.00 | −0.127 | 10.80 | 0.60 |
| ATOM | 3874 | HE1 | PHE | 236 | −15.333 | 6.121 | 35.368 | 1.00 | 0.00 | 0.127 | 0.00 | 0.00 |
| ATOM | 3875 | CE2 | PHE | 236 | −14.438 | 4.035 | 37.849 | 1.00 | 0.00 | −0.127 | 10.80 | 0.60 |
| ATOM | 3876 | HE2 | PHE | 236 | −14.861 | 3.495 | 38.696 | 1.00 | 0.00 | 0.127 | 0.00 | 0.00 |
| ATOM | 3877 | CZ | PHE | 236 | −15.239 | 4.829 | 37.066 | 1.00 | 0.00 | −0.127 | 10.80 | 0.60 |
| ATOM | 3878 | HZ | PHE | 236 | −16.300 | 4.928 | 37.293 | 1.00 | 0.00 | 0.127 | 0.00 | 0.00 |
| ATOM | 3879 | N | SER | 237 | −10.551 | 7.201 | 38.535 | 1.00 | 0.00 | −0.650 | 9.00 | −17.40 |
| ATOM | 3880 | HN | SER | 237 | −10.352 | 7.933 | 37.838 | 1.00 | 0.00 | 0.440 | 0.00 | 0.00 |
| ATOM | 3881 | CA | SER | 237 | −10.718 | 7.614 | 39.892 | 1.00 | 0.00 | 0.158 | 9.40 | 4.00 |
| ATOM | 3882 | HA | SER | 237 | −11.779 | 7.496 | 40.108 | 1.00 | 0.00 | 0.053 | 0.00 | 0.00 |
| ATOM | 3883 | C | SER | 237 | −10.270 | 9.034 | 39.941 | 1.00 | 0.00 | 0.396 | 9.82 | 4.00 |
| ATOM | 3884 | O | SER | 237 | −11.039 | 9.961 | 39.695 | 1.00 | 0.00 | −0.396 | 8.17 | −17.40 |
| ATOM | 3885 | CB | SER | 237 | −9.831 | 6.875 | 40.917 | 1.00 | 0.00 | 0.007 | 12.77 | 4.00 |
| ATOM | 3886 | HB1 | SER | 237 | −9.740 | 7.435 | 41.847 | 1.00 | 0.00 | 0.053 | 0.00 | 0.00 |
| ATOM | 3887 | HB2 | SER | 237 | −8.822 | 6.717 | 40.535 | 1.00 | 0.00 | 0.053 | 0.00 | 0.00 |
| ATOM | 3888 | OG | SER | 237 | −10.361 | 5.599 | 41.244 | 1.00 | 0.00 | −0.537 | 11.04 | −17.40 |
| ATOM | 3889 | HG | SER | 237 | −11.361 | 5.689 | 41.469 | 1.00 | 0.00 | 0.424 | 0.00 | 0.00 |
| ATOM | 3890 | N | GLY | 238 | −8.982 | 9.228 | 40.269 | 1.00 | 0.00 | −0.650 | 9.00 | −17.40 |
| ATOM | 3891 | HN | GLY | 238 | −8.380 | 8.407 | 40.433 | 1.00 | 0.00 | 0.440 | 0.00 | 0.00 |
| ATOM | 3892 | CA | GLY | 238 | −8.418 | 10.535 | 40.398 | 1.00 | 0.00 | 0.105 | 9.40 | 4.00 |
| ATOM | 3893 | HA1 | GLY | 238 | −9.231 | 11.225 | 40.174 | 1.00 | 0.00 | 0.053 | 0.00 | 0.00 |
| ATOM | 3894 | HA2 | GLY | 238 | −7.608 | 10.574 | 39.668 | 1.00 | 0.00 | 0.053 | 0.00 | 0.00 |
| ATOM | 3895 | C | GLY | 238 | −7.943 | 10.624 | 41.807 | 1.00 | 0.00 | 0.396 | 9.82 | 4.00 |
| ATOM | 3896 | O | GLY | 238 | −6.810 | 11.017 | 42.077 | 1.00 | 0.00 | −0.396 | 8.17 | −17.40 |
| ATOM | 3897 | N | SER | 239 | −8.829 | 10.254 | 42.748 | 1.00 | 0.00 | −0.650 | 9.00 | −17.40 |
| ATOM | 3898 | HN | SER | 239 | −9.774 | 9.964 | 42.459 | 1.00 | 0.00 | 0.440 | 0.00 | 0.00 |
| ATOM | 3899 | CA | SER | 239 | −8.495 | 10.252 | 44.139 | 1.00 | 0.00 | 0.158 | 9.40 | 4.00 |
| ATOM | 3900 | HA | SER | 239 | −8.063 | 11.224 | 44.375 | 1.00 | 0.00 | 0.053 | 0.00 | 0.00 |
| ATOM | 3901 | C | SER | 239 | −7.509 | 9.151 | 44.365 | 1.00 | 0.00 | 0.396 | 9.82 | 4.00 |
| ATOM | 3902 | O | SER | 239 | −6.583 | 9.281 | 45.164 | 1.00 | 0.00 | −0.396 | 8.17 | −17.40 |
| ATOM | 3903 | CB | SER | 239 | −9.706 | 9.980 | 45.044 | 1.00 | 0.00 | 0.007 | 12.77 | 4.00 |
| ATOM | 3904 | HB1 | SER | 239 | −10.138 | 9.007 | 44.809 | 1.00 | 0.00 | 0.053 | 0.00 | 0.00 |
| ATOM | 3905 | HB2 | SER | 239 | −10.465 | 10.747 | 44.895 | 1.00 | 0.00 | 0.053 | 0.00 | 0.00 |
| ATOM | 3906 | OG | SER | 239 | −9.309 | 9.988 | 46.408 | 1.00 | 0.00 | −0.537 | 11.04 | −17.40 |
| ATOM | 3907 | HG | SER | 239 | −9.207 | 10.960 | 46.730 | 1.00 | 0.00 | 0.424 | 0.00 | 0.00 |
| ATOM | 3908 | N | ALA | 240 | −7.690 | 8.034 | 43.637 | 1.00 | 0.00 | −0.650 | 9.00 | −17.40 |
| ATOM | 3909 | HN | ALA | 240 | −8.445 | 8.022 | 42.937 | 1.00 | 0.00 | 0.440 | 0.00 | 0.00 |
| ATOM | 3910 | CA | ALA | 240 | −6.882 | 6.860 | 43.788 | 1.00 | 0.00 | 0.158 | 9.40 | 4.00 |
| ATOM | 3911 | HA | ALA | 240 | −6.914 | 6.514 | 44.821 | 1.00 | 0.00 | 0.053 | 0.00 | 0.00 |
| ATOM | 3912 | C | ALA | 240 | −5.463 | 7.164 | 43.423 | 1.00 | 0.00 | 0.396 | 9.82 | 4.00 |
| ATOM | 3913 | O | ALA | 240 | −4.543 | 6.628 | 44.043 | 1.00 | 0.00 | −0.396 | 8.17 | −17.40 |
| ATOM | 3914 | CB | ALA | 240 | −7.347 | 5.699 | 42.892 | 1.00 | 0.00 | −0.159 | 16.15 | 4.00 |
| ATOM | 3915 | HB1 | ALA | 240 | −6.698 | 4.836 | 43.048 | 1.00 | 0.00 | 0.053 | 0.00 | 0.00 |
| ATOM | 3916 | HB2 | ALA | 240 | −8.373 | 5.432 | 43.145 | 1.00 | 0.00 | 0.053 | 0.00 | 0.00 |
| ATOM | 3917 | HB3 | ALA | 240 | −7.299 | 6.004 | 41.846 | 1.00 | 0.00 | 0.053 | 0.00 | 0.00 |
| ATOM | 3918 | N | TYR | 241 | −5.271 | 8.029 | 42.405 | 1.00 | 0.00 | −0.650 | 9.00 | −17.40 |
| ATOM | 3919 | HN | TYR | 241 | −6.111 | 8.460 | 41.993 | 1.00 | 0.00 | 0.440 | 0.00 | 0.00 |
| ATOM | 3920 | CA | TYR | 241 | −3.999 | 8.406 | 41.839 | 1.00 | 0.00 | 0.158 | 9.40 | 4.00 |
| ATOM | 3921 | HA | TYR | 241 | −3.656 | 7.691 | 41.090 | 1.00 | 0.00 | 0.053 | 0.00 | 0.00 |
| ATOM | 3922 | C | TYR | 241 | −2.920 | 8.493 | 42.883 | 1.00 | 0.00 | 0.396 | 9.82 | 4.00 |
| ATOM | 3923 | O | TYR | 241 | −3.178 | 8.769 | 44.051 | 1.00 | 0.00 | −0.396 | 8.17 | −17.40 |
| ATOM | 3924 | CB | TYR | 241 | −4.071 | 9.747 | 41.082 | 1.00 | 0.00 | −0.106 | 12.77 | 4.00 |
| ATOM | 3925 | HB1 | TYR | 241 | −4.381 | 10.530 | 41.773 | 1.00 | 0.00 | 0.053 | 0.00 | 0.00 |
| ATOM | 3926 | HB2 | TYR | 241 | −4.795 | 9.660 | 40.271 | 1.00 | 0.00 | 0.053 | 0.00 | 0.00 |
| ATOM | 3927 | CG | TYR | 241 | −2.725 | 10.066 | 40.526 | 1.00 | 0.00 | 0.000 | 7.26 | 0.60 |
| ATOM | 3928 | CD1 | TYR | 241 | −1.820 | 10.793 | 41.264 | 1.00 | 0.00 | −0.127 | 10.80 | 0.60 |
| ATOM | 3929 | HD1 | TYR | 241 | −2.090 | 11.138 | 42.261 | 1.00 | 0.00 | 0.127 | 0.00 | 0.00 |
| ATOM | 3930 | CD2 | TYR | 241 | −2.368 | 9.638 | 39.267 | 1.00 | 0.00 | −0.127 | 10.80 | 0.60 |
| ATOM | 3931 | HD2 | TYR | 241 | −3.076 | 9.062 | 38.672 | 1.00 | 0.00 | 0.127 | 0.00 | 0.00 |
| ATOM | 3932 | CE1 | TYR | 241 | −0.574 | 11.089 | 40.753 | 1.00 | 0.00 | −0.127 | 10.80 | 0.60 |
| ATOM | 3933 | HE1 | TYR | 241 | 0.136 | 11.666 | 41.344 | 1.00 | 0.00 | 0.127 | 0.00 | 0.00 |
| ATOM | 3934 | CE2 | TYR | 241 | −1.130 | 9.929 | 38.754 | 1.00 | 0.00 | −0.127 | 10.80 | 0.60 |
| ATOM | 3935 | HE2 | TYR | 241 | −0.859 | 9.583 | 37.756 | 1.00 | 0.00 | 0.127 | 0.00 | 0.00 |
| ATOM | 3936 | CZ | TYR | 241 | −0.231 | 10.654 | 39.495 | 1.00 | 0.00 | 0.026 | 7.26 | 0.60 |
| ATOM | 3937 | OH | TYR | 241 | 1.044 | 10.957 | 38.970 | 1.00 | 0.00 | −0.451 | 10.94 | −17.40 |
| ATOM | 3938 | HH | TYR | 241 | 1.562 | 11.540 | 39.642 | 1.00 | 0.00 | 0.424 | 0.00 | 0.00 |
| ATOM | 3939 | N | PHE | 242 | −1.667 | 8.233 | 42.445 | 1.00 | 0.00 | −0.650 | 9.00 | −17.40 |
| ATOM | 3940 | HN | PHE | 242 | −1.555 | 8.012 | 41.445 | 1.00 | 0.00 | 0.440 | 0.00 | 0.00 |
| ATOM | 3941 | CA | PHE | 242 | −0.475 | 8.237 | 43.253 | 1.00 | 0.00 | 0.158 | 9.40 | 4.00 |
| ATOM | 3942 | HA | PHE | 242 | −0.629 | 7.515 | 44.055 | 1.00 | 0.00 | 0.053 | 0.00 | 0.00 |
| ATOM | 3943 | C | PHE | 242 | −0.307 | 9.624 | 43.780 | 1.00 | 0.00 | 0.396 | 9.82 | 4.00 |
| ATOM | 3944 | O | PHE | 242 | 0.436 | 10.434 | 43.228 | 1.00 | 0.00 | −0.396 | 8.17 | −17.40 |
| ATOM | 3945 | CB | PHE | 242 | 0.768 | 7.893 | 42.406 | 1.00 | 0.00 | −0.106 | 12.77 | 4.00 |
| ATOM | 3946 | HB1 | PHE | 242 | 0.922 | 8.625 | 41.613 | 1.00 | 0.00 | 0.053 | 0.00 | 0.00 |
| ATOM | 3947 | HB2 | PHE | 242 | 0.668 | 6.915 | 41.934 | 1.00 | 0.00 | 0.053 | 0.00 | 0.00 |
| ATOM | 3948 | CG | PHE | 242 | 2.002 | 7.866 | 43.241 | 1.00 | 0.00 | 0.000 | 7.26 | 0.60 |
| ATOM | 3949 | CD1 | PHE | 242 | 2.403 | 6.700 | 43.853 | 1.00 | 0.00 | −0.127 | 10.80 | 0.60 |
| ATOM | 3950 | HD1 | PHE | 242 | 1.808 | 5.794 | 43.729 | 1.00 | 0.00 | 0.127 | 0.00 | 0.00 |
| ATOM | 3951 | CD2 | PHE | 242 | 2.769 | 8.997 | 43.402 | 1.00 | 0.00 | −0.127 | 10.80 | 0.60 |
| ATOM | 3952 | HD2 | PHE | 242 | 2.467 | 9.926 | 42.918 | 1.00 | 0.00 | 0.127 | 0.00 | 0.00 |
| ATOM | 3953 | CE1 | PHE | 242 | 3.545 | 6.664 | 44.619 | 1.00 | 0.00 | −0.127 | 10.80 | 0.60 |
| ATOM | 3954 | HE1 | PHE | 242 | 3.849 | 5.734 | 45.100 | 1.00 | 0.00 | 0.127 | 0.00 | 0.00 |
| ATOM | 3955 | CE2 | PHE | 242 | 3.912 | 8.968 | 44.167 | 1.00 | 0.00 | −0.127 | 10.80 | 0.60 |
| ATOM | 3956 | HE2 | PHE | 242 | 4.509 | 9.871 | 44.289 | 1.00 | 0.00 | 0.127 | 0.00 | 0.00 |
| ATOM | 3957 | CZ | PHE | 242 | 4.301 | 7.800 | 44.777 | 1.00 | 0.00 | −0.127 | 10.80 | 0.60 |
| ATOM | 3958 | HZ | PHE | 242 | 5.206 | 7.774 | 45.384 | 1.00 | 0.00 | 0.127 | 0.00 | 0.00 |
| ATOM | 3959 | N | VAL | 243 | −0.980 | 9.918 | 44.904 | 1.00 | 0.00 | −0.650 | 9.00 | −17.40 |
| ATOM | 3960 | HN | VAL | 243 | −1.500 | 9.174 | 45.391 | 1.00 | 0.00 | 0.440 | 0.00 | 0.00 |
| ATOM | 3961 | CA | VAL | 243 | −0.987 | 11.244 | 45.435 | 1.00 | 0.00 | 0.158 | 9.40 | 4.00 |
| ATOM | 3962 | HA | VAL | 243 | −1.414 | 11.938 | 44.712 | 1.00 | 0.00 | 0.053 | 0.00 | 0.00 |
| ATOM | 3963 | C | VAL | 243 | 0.397 | 11.680 | 45.751 | 1.00 | 0.00 | 0.396 | 9.82 | 4.00 |
| ATOM | 3964 | O | VAL | 243 | 1.341 | 10.891 | 45.789 | 1.00 | 0.00 | −0.396 | 8.17 | −17.40 |
| ATOM | 3965 | CB | VAL | 243 | −1.799 | 11.382 | 46.689 | 1.00 | 0.00 | −0.053 | 9.40 | 4.00 |
| ATOM | 3966 | HB | VAL | 243 | −1.706 | 12.405 | 47.051 | 1.00 | 0.00 | 0.053 | 0.00 | 0.00 |
| ATOM | 3967 | CG1 | VAL | 243 | −3.267 | 11.061 | 46.360 | 1.00 | 0.00 | −0.159 | 16.15 | 4.00 |
| ATOM | 3968 | HG1 | VAL | 243 | −3.871 | 11.156 | 47.262 | 1.00 | 0.00 | 0.053 | 0.00 | 0.00 |
| ATOM | 3969 | HG1 | VAL | 243 | −3.630 | 11.756 | 45.603 | 1.00 | 0.00 | 0.053 | 0.00 | 0.00 |
| ATOM | 3970 | HG1 | VAL | 243 | −3.340 | 10.041 | 45.980 | 1.00 | 0.00 | 0.053 | 0.00 | 0.00 |
| ATOM | 3971 | CG2 | VAL | 243 | −1.187 | 10.478 | 47.772 | 1.00 | 0.00 | −0.159 | 16.15 | 4.00 |
| ATOM | 3972 | HG2 | VAL | 243 | −1.766 | 10.567 | 48.690 | 1.00 | 0.00 | 0.053 | 0.00 | 0.00 |
| ATOM | 3973 | HG2 | VAL | 243 | −1.202 | 9.442 | 47.432 | 1.00 | 0.00 | 0.053 | 0.00 | 0.00 |
| ATOM | 3974 | HG2 | VAL | 243 | −0.157 | 10.782 | 47.961 | 1.00 | 0.00 | 0.053 | 0.00 | 0.00 |
| ATOM | 3975 | N | VAL | 244 | 0.527 | 12.999 | 45.978 | 1.00 | 0.00 | −0.650 | 9.00 | −17.40 |
| ATOM | 3976 | HN | VAL | 244 | −0.296 | 13.610 | 45.886 | 1.00 | 0.00 | 0.440 | 0.00 | 0.00 |
| ATOM | 3977 | CA | VAL | 244 | 1.782 | 13.566 | 46.342 | 1.00 | 0.00 | 0.158 | 9.40 | 4.00 |
| ATOM | 3978 | HA | VAL | 244 | 2.444 | 13.316 | 45.513 | 1.00 | 0.00 | 0.053 | 0.00 | 0.00 |
| ATOM | 3979 | C | VAL | 244 | 2.135 | 12.898 | 47.618 | 1.00 | 0.00 | 0.396 | 9.82 | 4.00 |
| ATOM | 3980 | O | VAL | 244 | 3.308 | 12.725 | 47.936 | 1.00 | 0.00 | −0.396 | 8.17 | −17.40 |
| ATOM | 3981 | CB | VAL | 244 | 1.702 | 15.041 | 46.604 | 1.00 | 0.00 | −0.053 | 9.40 | 4.00 |
| ATOM | 3982 | HB | VAL | 244 | 2.679 | 15.387 | 46.939 | 1.00 | 0.00 | 0.053 | 0.00 | 0.00 |
| ATOM | 3983 | CG1 | VAL | 244 | 1.302 | 15.750 | 45.298 | 1.00 | 0.00 | −0.159 | 16.15 | 4.00 |
| ATOM | 3984 | HG1 | VAL | 244 | 1.239 | 16.824 | 45.471 | 1.00 | 0.00 | 0.053 | 0.00 | 0.00 |
| ATOM | 3985 | HG1 | VAL | 244 | 2.050 | 15.549 | 44.531 | 1.00 | 0.00 | 0.053 | 0.00 | 0.00 |
| ATOM | 3986 | HG1 | VAL | 244 | 0.332 | 15.379 | 44.964 | 1.00 | 0.00 | 0.053 | 0.00 | 0.00 |
| ATOM | 3987 | CG2 | VAL | 244 | 0.725 | 15.278 | 47.768 | 1.00 | 0.00 | −0.159 | 16.15 | 4.00 |
| ATOM | 3988 | HG2 | VAL | 244 | 0.654 | 16.346 | 47.972 | 1.00 | 0.00 | 0.053 | 0.00 | 0.00 |
| ATOM | 3989 | HG2 | VAL | 244 | −0.259 | 14.894 | 47.500 | 1.00 | 0.00 | 0.053 | 0.00 | 0.00 |
| ATOM | 3990 | HG2 | VAL | 244 | 1.086 | 14.761 | 48.657 | 1.00 | 0.00 | 0.053 | 0.00 | 0.00 |
| ATOM | 3991 | N | SER | 245 | 1.104 | 12.483 | 48.376 | 1.00 | 0.00 | −0.650 | 9.00 | −17.40 |
| ATOM | 3992 | HN | SER | 245 | 0.137 | 12.636 | 48.053 | 1.00 | 0.00 | 0.440 | 0.00 | 0.00 |
| ATOM | 3993 | CA | SER | 245 | 1.341 | 11.831 | 49.626 | 1.00 | 0.00 | 0.158 | 9.40 | 4.00 |
| ATOM | 3994 | HA | SER | 245 | 1.881 | 12.538 | 50.254 | 1.00 | 0.00 | 0.053 | 0.00 | 0.00 |
| ATOM | 3995 | C | SER | 245 | 2.150 | 10.616 | 49.334 | 1.00 | 0.00 | 0.396 | 9.82 | 4.00 |
| ATOM | 3996 | O | SER | 245 | 3.122 | 10.327 | 50.029 | 1.00 | 0.00 | −0.396 | 8.17 | −17.40 |
| ATOM | 3997 | CB | SER | 245 | 0.045 | 11.374 | 50.317 | 1.00 | 0.00 | 0.007 | 12.77 | 4.00 |
| ATOM | 3998 | HB1 | SER | 245 | −0.492 | 10.673 | 49.678 | 1.00 | 0.00 | 0.053 | 0.00 | 0.00 |
| ATOM | 3999 | HB2 | SER | 245 | −0.596 | 12.232 | 50.515 | 1.00 | 0.00 | 0.053 | 0.00 | 0.00 |
| ATOM | 4000 | OG | SER | 245 | 0.348 | 10.734 | 51.547 | 1.00 | 0.00 | −0.537 | 11.04 | −17.40 |
| ATOM | 4001 | HG | SER | 245 | −0.016 | 11.298 | 52.327 | 1.00 | 0.00 | 0.424 | 0.00 | 0.00 |
| ATOM | 4002 | N | ARG | 246G | 1.780 | 9.877 | 48.271 | 1.00 | 0.00 | −0.650 | 9.00 | −17.40 |
| ATOM | 4003 | HN | ARG | 246G | 0.962 | 10.157 | 47.710 | 1.00 | 0.00 | 0.440 | 0.00 | 0.00 |
| ATOM | 4004 | CA | ARG | 246G | 2.519 | 8.702 | 47.917 | 1.00 | 0.00 | 0.158 | 9.40 | 4.00 |
| ATOM | 4005 | HA | ARG | 246G | 2.527 | 8.051 | 48.791 | 1.00 | 0.00 | 0.053 | 0.00 | 0.00 |
| ATOM | 4006 | C | ARG | 246G | 3.894 | 9.143 | 47.545 | 1.00 | 0.00 | 0.396 | 9.82 | 4.00 |
| ATOM | 4007 | O | ARG | 246G | 4.880 | 8.473 | 47.848 | 1.00 | 0.00 | −0.396 | 8.17 | −17.40 |
| ATOM | 4008 | CB | ARG | 246G | 1.926 | 7.944 | 46.722 | 1.00 | 0.00 | −0.106 | 12.77 | 4.00 |
| ATOM | 4009 | HB1 | ARG | 246G | 2.579 | 7.164 | 46.329 | 1.00 | 0.00 | 0.053 | 0.00 | 0.00 |
| ATOM | 4010 | HB2 | ARG | 246G | 1.703 | 8.580 | 45.866 | 1.00 | 0.00 | 0.053 | 0.00 | 0.00 |
| ATOM | 4011 | CG | ARG | 246G | 0.610 | 7.233 | 47.037 | 1.00 | 0.00 | −0.106 | 12.77 | 4.00 |
| ATOM | 4012 | HG1 | ARG | 246G | −0.234 | 7.914 | 47.137 | 1.00 | 0.00 | 0.053 | 0.00 | 0.00 |
| ATOM | 4013 | HG2 | ARG | 246G | 0.638 | 6.667 | 47.968 | 1.00 | 0.00 | 0.053 | 0.00 | 0.00 |
| ATOM | 4014 | CD | ARG | 246G | 0.195 | 6.227 | 45.963 | 1.00 | 0.00 | 0.374 | 12.77 | 4.00 |
| ATOM | 4015 | HD1 | ARG | 246G | 0.180 | 6.759 | 45.012 | 1.00 | 0.00 | 0.053 | 0.00 | 0.00 |
| ATOM | 4016 | HD2 | ARG | 246G | −0.794 | 5.857 | 46.233 | 1.00 | 0.00 | 0.053 | 0.00 | 0.00 |
| ATOM | 4017 | NE | ARG | 246G | 1.210 | 5.136 | 45.967 | 1.00 | 0.00 | −0.819 | 9.00 | −24.67 |
| ATOM | 4018 | HE | ARG | 246G | 2.000 | 5.165 | 45.307 | 1.00 | 0.00 | 0.407 | 0.00 | 0.00 |
| ATOM | 4019 | CZ | ARG | 246G | 1.092 | 4.098 | 46.846 | 1.00 | 0.00 | 0.796 | 6.95 | 4.00 |
| ATOM | 4020 | NH1 | ARG | 246G | 0.040 | 4.057 | 47.716 | 1.00 | 0.00 | −0.746 | 9.00 | −24.67 |
| ATOM | 4021 | HH1 | ARG | 246G | −0.049 | 3.274 | 48.379 | 1.00 | 0.00 | 0.407 | 0.00 | 0.00 |
| ATOM | 4022 | HH1 | ARG | 246G | −0.663 | 4.809 | 47.708 | 1.00 | 0.00 | 0.407 | 0.00 | 0.00 |
| ATOM | 4023 | NH2 | ARG | 246G | 2.026 | 3.102 | 46.857 | 1.00 | 0.00 | −0.746 | 9.00 | −24.67 |
| ATOM | 4024 | HH2 | ARG | 246G | 1.936 | 2.319 | 47.520 | 1.00 | 0.00 | 0.407 | 0.00 | 0.00 |
| ATOM | 4025 | HH2 | ARG | 246G | 2.820 | 3.133 | 46.201 | 1.00 | 0.00 | 0.407 | 0.00 | 0.00 |
| ATOM | 4026 | N | GLU | 247 | 3.987 | 10.309 | 46.884 | 1.00 | 0.00 | −0.650 | 9.00 | −17.40 |
| ATOM | 4027 | HN | GLU | 247 | 3.128 | 10.834 | 46.664 | 1.00 | 0.00 | 0.440 | 0.00 | 0.00 |
| ATOM | 4028 | CA | GLU | 247 | 5.253 | 10.837 | 46.478 | 1.00 | 0.00 | 0.158 | 9.40 | 4.00 |
| ATOM | 4029 | HA | GLU | 247 | 5.693 | 10.122 | 45.783 | 1.00 | 0.00 | 0.053 | 0.00 | 0.00 |
| ATOM | 4030 | C | GLU | 247 | 6.083 | 10.990 | 47.708 | 1.00 | 0.00 | 0.396 | 9.82 | 4.00 |
| ATOM | 4031 | O | GLU | 247 | 7.250 | 10.601 | 47.736 | 1.00 | 0.00 | −0.396 | 8.17 | −17.40 |
| ATOM | 4032 | CB | GLU | 247 | 5.093 | 12.227 | 45.832 | 1.00 | 0.00 | −0.106 | 12.77 | 4.00 |
| ATOM | 4033 | HB1 | GLU | 247 | 4.420 | 12.815 | 46.456 | 1.00 | 0.00 | 0.053 | 0.00 | 0.00 |
| ATOM | 4034 | HB2 | GLU | 247 | 4.675 | 12.090 | 44.834 | 1.00 | 0.00 | 0.053 | 0.00 | 0.00 |
| ATOM | 4035 | CG | GLU | 247 | 6.388 | 13.024 | 45.679 | 1.00 | 0.00 | −0.106 | 12.77 | 4.00 |
| ATOM | 4036 | HG1 | GLU | 247 | 6.263 | 13.701 | 44.834 | 1.00 | 0.00 | 0.053 | 0.00 | 0.00 |
| ATOM | 4037 | HG2 | GLU | 247 | 7.195 | 12.314 | 45.498 | 1.00 | 0.00 | 0.053 | 0.00 | 0.00 |
| ATOM | 4038 | CD | GLU | 247 | 6.614 | 13.796 | 46.973 | 1.00 | 0.00 | 0.399 | 9.82 | 4.00 |
| ATOM | 4039 | OE1 | GLU | 247 | 5.926 | 14.833 | 47.169 | 1.00 | 0.00 | −0.396 | 8.17 | −18.95 |
| ATOM | 4040 | OE2 | GLU | 247 | 7.472 | 13.356 | 47.785 | 1.00 | 0.00 | −0.427 | 8.17 | −18.95 |
| ATOM | 4042 | N | TYR | 248 | 5.482 | 11.550 | 48.769 | 1.00 | 0.00 | −0.650 | 9.00 | −17.40 |
| ATOM | 4043 | HN | TYR | 248 | 4.494 | 11.833 | 48.690 | 1.00 | 0.00 | 0.440 | 0.00 | 0.00 |
| ATOM | 4044 | CA | TYR | 248 | 6.168 | 11.766 | 50.005 | 1.00 | 0.00 | 0.158 | 9.40 | 4.00 |
| ATOM | 4045 | HA | TYR | 248 | 7.115 | 12.266 | 49.803 | 1.00 | 0.00 | 0.053 | 0.00 | 0.00 |
| ATOM | 4046 | C | TYR | 248 | 6.419 | 10.448 | 50.660 | 1.00 | 0.00 | 0.396 | 9.82 | 4.00 |
| ATOM | 4047 | O | TYR | 248 | 7.362 | 10.305 | 51.437 | 1.00 | 0.00 | −0.396 | 8.17 | −17.40 |
| ATOM | 4048 | CB | TYR | 248 | 5.386 | 12.653 | 50.989 | 1.00 | 0.00 | −0.106 | 12.77 | 4.00 |
| ATOM | 4049 | HB1 | TYR | 248 | 5.929 | 12.629 | 51.933 | 1.00 | 0.00 | 0.053 | 0.00 | 0.00 |
| ATOM | 4050 | HB2 | TYR | 248 | 4.390 | 12.219 | 51.078 | 1.00 | 0.00 | 0.053 | 0.00 | 0.00 |
| ATOM | 4051 | CG | TYR | 248 | 5.350 | 14.021 | 50.398 | 1.00 | 0.00 | 0.000 | 7.26 | 0.60 |
| ATOM | 4052 | CD1 | TYR | 248 | 6.469 | 14.819 | 50.437 | 1.00 | 0.00 | −0.127 | 10.80 | 0.60 |
| ATOM | 4053 | HD1 | TYR | 248 | 7.381 | 14.445 | 50.902 | 1.00 | 0.00 | 0.127 | 0.00 | 0.00 |
| ATOM | 4054 | CD2 | TYR | 248 | 4.205 | 14.510 | 49.816 | 1.00 | 0.00 | −0.127 | 10.80 | 0.60 |
| ATOM | 4055 | HD2 | TYR | 248 | 3.308 | 13.890 | 49.785 | 1.00 | 0.00 | 0.127 | 0.00 | 0.00 |
| ATOM | 4056 | CE1 | TYR | 248 | 6.450 | 16.083 | 49.895 | 1.00 | 0.00 | −0.127 | 10.80 | 0.60 |
| ATOM | 4057 | HE1 | TYR | 248 | 7.344 | 16.704 | 49.929 | 1.00 | 0.00 | 0.127 | 0.00 | 0.00 |
| ATOM | 4058 | CE2 | TYR | 248 | 4.178 | 15.773 | 49.272 | 1.00 | 0.00 | −0.127 | 10.80 | 0.60 |
| ATOM | 4059 | HE2 | TYR | 248 | 3.264 | 16.148 | 48.810 | 1.00 | 0.00 | 0.127 | 0.00 | 0.00 |
| ATOM | 4060 | CZ | TYR | 248 | 5.303 | 16.561 | 49.311 | 1.00 | 0.00 | 0.026 | 7.26 | 0.60 |
| ATOM | 4061 | OH | TYR | 248 | 5.279 | 17.858 | 48.753 | 1.00 | 0.00 | −0.451 | 10.94 | −17.40 |
| ATOM | 4062 | HH | TYR | 248 | 6.164 | 18.339 | 48.963 | 1.00 | 0.00 | 0.424 | 0.00 | 0.00 |
| ATOM | 4063 | N | VAL | 249 | 5.562 | 9.450 | 50.379 | 1.00 | 0.00 | −0.650 | 9.00 | −17.40 |
| ATOM | 4064 | HN | VAL | 249 | 4.791 | 9.615 | 49.716 | 1.00 | 0.00 | 0.440 | 0.00 | 0.00 |
| ATOM | 4065 | CA | VAL | 249 | 5.704 | 8.159 | 50.987 | 1.00 | 0.00 | 0.158 | 9.40 | 4.00 |
| ATOM | 4066 | HA | VAL | 249 | 5.689 | 8.273 | 52.070 | 1.00 | 0.00 | 0.053 | 0.00 | 0.00 |
| ATOM | 4067 | C | VAL | 249 | 7.002 | 7.559 | 50.556 | 1.00 | 0.00 | 0.396 | 9.82 | 4.00 |
| ATOM | 4068 | O | VAL | 249 | 7.716 | 6.969 | 51.365 | 1.00 | 0.00 | −0.396 | 8.17 | −17.40 |
| ATOM | 4069 | CB | VAL | 249 | 4.620 | 7.201 | 50.587 | 1.00 | 0.00 | −0.053 | 9.40 | 4.00 |
| ATOM | 4070 | HB | VAL | 249 | 4.598 | 7.139 | 49.498 | 1.00 | 0.00 | 0.053 | 0.00 | 0.00 |
| ATOM | 4071 | CG1 | VAL | 249 | 4.937 | 5.826 | 51.198 | 1.00 | 0.00 | −0.159 | 16.15 | 4.00 |
| ATOM | 4072 | HG1 | VAL | 249 | 4.159 | 5.115 | 50.918 | 1.00 | 0.00 | 0.053 | 0.00 | 0.00 |
| ATOM | 4073 | HG1 | VAL | 249 | 5.899 | 5.475 | 50.825 | 1.00 | 0.00 | 0.053 | 0.00 | 0.00 |
| ATOM | 4074 | HG1 | VAL | 249 | 4.977 | 5.910 | 52.284 | 1.00 | 0.00 | 0.053 | 0.00 | 0.00 |
| ATOM | 4075 | CG2 | VAL | 249 | 3.264 | 7.776 | 51.031 | 1.00 | 0.00 | −0.159 | 16.15 | 4.00 |
| ATOM | 4076 | HG2 | VAL | 249 | 2.466 | 7.089 | 50.746 | 1.00 | 0.00 | 0.053 | 0.00 | 0.00 |
| ATOM | 4077 | HG2 | VAL | 249 | 3.261 | 7.906 | 52.113 | 1.00 | 0.00 | 0.053 | 0.00 | 0.00 |
| ATOM | 4078 | HG2 | VAL | 249 | 3.102 | 8.739 | 50.548 | 1.00 | 0.00 | 0.053 | 0.00 | 0.00 |
| ATOM | 4079 | N | GLY | 250 | 7.353 | 7.712 | 49.266 | 1.00 | 0.00 | −0.650 | 9.00 | −17.40 |
| ATOM | 4080 | HN | GLY | 250 | 6.754 | 8.266 | 48.637 | 1.00 | 0.00 | 0.440 | 0.00 | 0.00 |
| ATOM | 4081 | CA | GLY | 250 | 8.551 | 7.111 | 48.758 | 1.00 | 0.00 | 0.105 | 9.40 | 4.00 |
| ATOM | 4082 | HA1 | GLY | 250 | 8.646 | 7.338 | 47.696 | 1.00 | 0.00 | 0.053 | 0.00 | 0.00 |
| ATOM | 4083 | HA2 | GLY | 250 | 8.500 | 6.031 | 48.897 | 1.00 | 0.00 | 0.053 | 0.00 | 0.00 |
| ATOM | 4084 | C | GLY | 250 | 9.732 | 7.658 | 49.497 | 1.00 | 0.00 | 0.396 | 9.82 | 4.00 |
| ATOM | 4085 | O | GLY | 250 | 9.742 | 8.804 | 49.942 | 1.00 | 0.00 | −0.396 | 8.17 | −17.40 |
| ATOM | 4086 | N | TYR | 251 | 10.779 | 6.821 | 49.629 | 1.00 | 0.00 | −0.650 | 9.00 | −17.40 |
| ATOM | 4087 | HN | TYR | 251 | 10.715 | 5.877 | 49.220 | 1.00 | 0.00 | 0.440 | 0.00 | 0.00 |
| ATOM | 4088 | CA | TYR | 251 | 11.979 | 7.197 | 50.319 | 1.00 | 0.00 | 0.158 | 9.40 | 4.00 |
| ATOM | 4089 | HA | TYR | 251 | 11.718 | 7.657 | 51.272 | 1.00 | 0.00 | 0.053 | 0.00 | 0.00 |
| ATOM | 4090 | C | TYR | 251 | 12.745 | 8.171 | 49.485 | 1.00 | 0.00 | 0.396 | 9.82 | 4.00 |
| ATOM | 4091 | O | TYR | 251 | 12.521 | 8.297 | 48.282 | 1.00 | 0.00 | −0.396 | 8.17 | −17.40 |
| ATOM | 4092 | CB | TYR | 251 | 12.883 | 5.995 | 50.623 | 1.00 | 0.00 | −0.106 | 12.77 | 4.00 |
| ATOM | 4093 | HB1 | TYR | 251 | 12.494 | 5.418 | 51.462 | 1.00 | 0.00 | 0.053 | 0.00 | 0.00 |
| ATOM | 4094 | HB2 | TYR | 251 | 13.891 | 6.321 | 50.876 | 1.00 | 0.00 | 0.053 | 0.00 | 0.00 |
| ATOM | 4095 | CG | TYR | 251 | 12.865 | 5.207 | 49.366 | 1.00 | 0.00 | 0.000 | 7.26 | 0.60 |
| ATOM | 4096 | CD1 | TYR | 251 | 13.757 | 5.451 | 48.349 | 1.00 | 0.00 | −0.127 | 10.80 | 0.60 |
| ATOM | 4097 | HD1 | TYR | 251 | 14.508 | 6.232 | 48.459 | 1.00 | 0.00 | 0.127 | 0.00 | 0.00 |
| ATOM | 4098 | CD2 | TYR | 251 | 11.910 | 4.231 | 49.209 | 1.00 | 0.00 | −0.127 | 10.80 | 0.60 |
| ATOM | 4099 | HD2 | TYR | 251 | 11.191 | 4.046 | 50.007 | 1.00 | 0.00 | 0.127 | 0.00 | 0.00 |
| ATOM | 4100 | CE1 | TYR | 251 | 13.702 | 4.710 | 47.193 | 1.00 | 0.00 | −0.127 | 10.80 | 0.60 |
| ATOM | 4101 | HE1 | TYR | 251 | 14.413 | 4.899 | 46.389 | 1.00 | 0.00 | 0.127 | 0.00 | 0.00 |
| ATOM | 4102 | CE2 | TYR | 251 | 11.851 | 3.488 | 48.059 | 1.00 | 0.00 | −0.127 | 10.80 | 0.60 |
| ATOM | 4103 | HE2 | TYR | 251 | 11.095 | 2.710 | 47.946 | 1.00 | 0.00 | 0.127 | 0.00 | 0.00 |
| ATOM | 4104 | CZ | TYR | 251 | 12.749 | 3.729 | 47.051 | 1.00 | 0.00 | 0.026 | 7.26 | 0.60 |
| ATOM | 4105 | OH | TYR | 251 | 12.683 | 2.965 | 45.872 | 1.00 | 0.00 | −0.451 | 10.94 | −17.40 |
| ATOM | 4106 | HH | TYR | 251 | 12.139 | 3.475 | 45.161 | 1.00 | 0.00 | 0.424 | 0.00 | 0.00 |
| ATOM | 4107 | N | VAL | 252 | 13.677 | 8.899 | 50.134 | 1.00 | 0.00 | −0.650 | 9.00 | −17.40 |
| ATOM | 4108 | HN | VAL | 252 | 13.830 | 8.735 | 51.139 | 1.00 | 0.00 | 0.440 | 0.00 | 0.00 |
| ATOM | 4109 | CA | VAL | 252 | 14.460 | 9.895 | 49.460 | 1.00 | 0.00 | 0.158 | 9.40 | 4.00 |
| ATOM | 4110 | HA | VAL | 252 | 13.939 | 10.163 | 48.540 | 1.00 | 0.00 | 0.053 | 0.00 | 0.00 |
| ATOM | 4111 | C | VAL | 252 | 15.795 | 9.304 | 49.166 | 1.00 | 0.00 | 0.396 | 9.82 | 4.00 |
| ATOM | 4112 | O | VAL | 252 | 16.356 | 8.580 | 49.981 | 1.00 | 0.00 | −0.396 | 8.17 | −17.40 |
| ATOM | 4113 | CB | VAL | 252 | 14.699 | 11.125 | 50.289 | 1.00 | 0.00 | −0.053 | 9.40 | 4.00 |
| ATOM | 4114 | HB | VAL | 252 | 15.226 | 10.832 | 51.196 | 1.00 | 0.00 | 0.053 | 0.00 | 0.00 |
| ATOM | 4115 | CG1 | VAL | 252 | 15.548 | 12.109 | 49.468 | 1.00 | 0.00 | −0.159 | 16.15 | 4.00 |
| ATOM | 4116 | HG1 | VAL | 252 | 15.731 | 13.008 | 50.055 | 1.00 | 0.00 | 0.053 | 0.00 | 0.00 |
| ATOM | 4117 | HG1 | VAL | 252 | 16.499 | 11.642 | 49.211 | 1.00 | 0.00 | 0.053 | 0.00 | 0.00 |
| ATOM | 4118 | HG1 | VAL | 252 | 15.016 | 12.374 | 48.554 | 1.00 | 0.00 | 0.053 | 0.00 | 0.00 |
| ATOM | 4119 | CG2 | VAL | 252 | 13.341 | 11.691 | 50.741 | 1.00 | 0.00 | −0.159 | 16.15 | 4.00 |
| ATOM | 4120 | HG2 | VAL | 252 | 13.501 | 12.584 | 51.344 | 1.00 | 0.00 | 0.053 | 0.00 | 0.00 |
| ATOM | 4121 | HG2 | VAL | 252 | 12.743 | 11.946 | 49.865 | 1.00 | 0.00 | 0.053 | 0.00 | 0.00 |
| ATOM | 4122 | HG2 | VAL | 252 | 12.814 | 10.943 | 51.333 | 1.00 | 0.00 | 0.053 | 0.00 | 0.00 |
| ATOM | 4123 | N | LEU | 253 | 16.335 | 9.587 | 47.966 | 1.00 | 0.00 | −0.650 | 9.00 | −17.40 |
| ATOM | 4124 | HN | LEU | 253 | 15.851 | 10.207 | 47.301 | 1.00 | 0.00 | 0.440 | 0.00 | 0.00 |
| ATOM | 4125 | CA | LEU | 253 | 17.597 | 9.003 | 47.639 | 1.00 | 0.00 | 0.158 | 9.40 | 4.00 |
| ATOM | 4126 | HA | LEU | 253 | 18.134 | 8.787 | 48.562 | 1.00 | 0.00 | 0.053 | 0.00 | 0.00 |
| ATOM | 4127 | C | LEU | 253 | 18.376 | 9.973 | 46.803 | 1.00 | 0.00 | 0.396 | 9.82 | 4.00 |
| ATOM | 4128 | O | LEU | 253 | 17.814 | 10.771 | 46.056 | 1.00 | 0.00 | −0.396 | 8.17 | −17.40 |
| ATOM | 4129 | CB | LEU | 253 | 17.408 | 7.625 | 46.967 | 1.00 | 0.00 | −0.106 | 12.77 | 4.00 |
| ATOM | 4130 | HB1 | LEU | 253 | 16.837 | 7.777 | 46.050 | 1.00 | 0.00 | 0.053 | 0.00 | 0.00 |
| ATOM | 4131 | HB2 | LEU | 253 | 16.866 | 6.986 | 47.664 | 1.00 | 0.00 | 0.053 | 0.00 | 0.00 |
| ATOM | 4132 | CG | LEU | 253 | 18.688 | 6.874 | 46.576 | 1.00 | 0.00 | −0.053 | 9.40 | 4.00 |
| ATOM | 4133 | HG | LEU | 253 | 19.414 | 6.873 | 47.388 | 1.00 | 0.00 | 0.053 | 0.00 | 0.00 |
| ATOM | 4134 | CD1 | LEU | 253 | 18.395 | 5.412 | 46.223 | 1.00 | 0.00 | −0.159 | 16.15 | 4.00 |
| ATOM | 4135 | HD1 | LEU | 253 | 19.323 | 4.910 | 45.951 | 1.00 | 0.00 | 0.053 | 0.00 | 0.00 |
| ATOM | 4136 | HD1 | LEU | 253 | 17.951 | 4.911 | 47.083 | 1.00 | 0.00 | 0.053 | 0.00 | 0.00 |
| ATOM | 4137 | HD1 | LEU | 253 | 17.701 | 5.372 | 45.382 | 1.00 | 0.00 | 0.053 | 0.00 | 0.00 |
| ATOM | 4138 | CD2 | LEU | 253 | 19.372 | 7.570 | 45.401 | 1.00 | 0.00 | −0.159 | 16.15 | 4.00 |
| ATOM | 4139 | HD2 | LEU | 253 | 20.278 | 7.026 | 45.134 | 1.00 | 0.00 | 0.053 | 0.00 | 0.00 |
| ATOM | 4140 | HD2 | LEU | 253 | 18.695 | 7.589 | 44.546 | 1.00 | 0.00 | 0.053 | 0.00 | 0.00 |
| ATOM | 4141 | HD2 | LEU | 253 | 19.630 | 8.590 | 45.683 | 1.00 | 0.00 | 0.053 | 0.00 | 0.00 |
| ATOM | 4142 | N | GLN | 254 | 19.717 | 9.918 | 46.934 | 1.00 | 0.00 | −0.650 | 9.00 | −17.40 |
| ATOM | 4143 | HN | GLN | 254 | 20.107 | 9.178 | 47.535 | 1.00 | 0.00 | 0.440 | 0.00 | 0.00 |
| ATOM | 4144 | CA | GLN | 254 | 20.642 | 10.816 | 46.296 | 1.00 | 0.00 | 0.158 | 9.40 | 4.00 |
| ATOM | 4145 | HA | GLN | 254 | 20.545 | 11.817 | 46.714 | 1.00 | 0.00 | 0.053 | 0.00 | 0.00 |
| ATOM | 4146 | C | GLN | 254 | 20.370 | 10.883 | 44.828 | 1.00 | 0.00 | 0.396 | 9.82 | 4.00 |
| ATOM | 4147 | O | GLN | 254 | 20.148 | 9.874 | 44.164 | 1.00 | 0.00 | −0.396 | 8.17 | −17.40 |
| ATOM | 4148 | CB | GLN | 254 | 22.103 | 10.357 | 46.444 | 1.00 | 0.00 | −0.106 | 12.77 | 4.00 |
| ATOM | 4149 | HB1 | GLN | 254 | 22.740 | 11.068 | 45.918 | 1.00 | 0.00 | 0.053 | 0.00 | 0.00 |
| ATOM | 4150 | HB2 | GLN | 254 | 22.195 | 9.363 | 46.006 | 1.00 | 0.00 | 0.053 | 0.00 | 0.00 |
| ATOM | 4151 | CG | GLN | 254 | 22.594 | 10.274 | 47.890 | 1.00 | 0.00 | −0.106 | 12.77 | 4.00 |
| ATOM | 4152 | HG1 | GLN | 254 | 23.411 | 9.554 | 47.930 | 1.00 | 0.00 | 0.053 | 0.00 | 0.00 |
| ATOM | 4153 | HG2 | GLN | 254 | 21.761 | 9.948 | 48.513 | 1.00 | 0.00 | 0.053 | 0.00 | 0.00 |
| ATOM | 4154 | CD | GLN | 254 | 23.071 | 11.654 | 48.308 | 1.00 | 0.00 | 0.396 | 9.82 | 4.00 |
| ATOM | 4155 | OE1 | GLN | 254 | 23.564 | 11.837 | 49.419 | 1.00 | 0.00 | −0.396 | 8.17 | −17.40 |
| ATOM | 4156 | NE2 | GLN | 254 | 22.933 | 12.649 | 47.392 | 1.00 | 0.00 | −0.879 | 13.25 | −17.40 |
| ATOM | 4157 | HE2 | GLN | 254 | 22.513 | 12.448 | 46.472 | 1.00 | 0.00 | 0.440 | 0.00 | 0.00 |
| ATOM | 4158 | HE2 | GLN | 254 | 23.247 | 13.603 | 47.617 | 1.00 | 0.00 | 0.440 | 0.00 | 0.00 |
| ATOM | 4159 | N | ASN | 255 | 20.396 | 12.105 | 44.270 | 1.00 | 0.00 | −0.650 | 9.00 | −17.40 |
| ATOM | 4160 | HN | ASN | 255 | 20.570 | 12.934 | 44.855 | 1.00 | 0.00 | 0.440 | 0.00 | 0.00 |
| ATOM | 4161 | CA | ASN | 255 | 20.181 | 12.256 | 42.863 | 1.00 | 0.00 | 0.158 | 9.40 | 4.00 |
| ATOM | 4162 | HA | ASN | 255 | 19.221 | 11.793 | 42.631 | 1.00 | 0.00 | 0.053 | 0.00 | 0.00 |
| ATOM | 4163 | C | ASN | 255 | 21.307 | 11.565 | 42.166 | 1.00 | 0.00 | 0.396 | 9.82 | 4.00 |
| ATOM | 4164 | O | ASN | 255 | 21.112 | 10.894 | 41.154 | 1.00 | 0.00 | −0.396 | 8.17 | −17.40 |
| ATOM | 4165 | CB | ASN | 255 | 20.170 | 13.728 | 42.413 | 1.00 | 0.00 | −0.106 | 12.77 | 4.00 |
| ATOM | 4166 | HB1 | ASN | 255 | 19.392 | 14.254 | 42.966 | 1.00 | 0.00 | 0.053 | 0.00 | 0.00 |
| ATOM | 4167 | HB2 | ASN | 255 | 19.963 | 13.762 | 41.343 | 1.00 | 0.00 | 0.053 | 0.00 | 0.00 |
| ATOM | 4168 | CG | ASN | 255 | 21.534 | 14.335 | 42.709 | 1.00 | 0.00 | 0.396 | 9.82 | 4.00 |
| ATOM | 4169 | OD1 | ASN | 255 | 22.072 | 14.176 | 43.804 | 1.00 | 0.00 | −0.396 | 8.17 | −17.40 |
| ATOM | 4170 | ND2 | ASN | 255 | 22.115 | 15.045 | 41.705 | 1.00 | 0.00 | −0.879 | 13.25 | −17.40 |
| ATOM | 4171 | HD2 | ASN | 255 | 21.629 | 15.153 | 40.803 | 1.00 | 0.00 | 0.440 | 0.00 | 0.00 |
| ATOM | 4172 | HD2 | ASN | 255 | 23.040 | 15.474 | 41.846 | 1.00 | 0.00 | 0.440 | 0.00 | 0.00 |
| ATOM | 4173 | N | GLU | 256 | 22.522 | 11.686 | 42.731 | 1.00 | 0.00 | −0.650 | 9.00 | −17.40 |
| ATOM | 4174 | HN | GLU | 256 | 22.614 | 12.196 | 43.620 | 1.00 | 0.00 | 0.440 | 0.00 | 0.00 |
| ATOM | 4175 | CA | GLU | 256 | 23.689 | 11.122 | 42.125 | 1.00 | 0.00 | 0.158 | 9.40 | 4.00 |
| ATOM | 4176 | HA | GLU | 256 | 23.808 | 11.559 | 41.133 | 1.00 | 0.00 | 0.053 | 0.00 | 0.00 |
| ATOM | 4177 | C | GLU | 256 | 23.502 | 9.647 | 42.025 | 1.00 | 0.00 | 0.396 | 9.82 | 4.00 |
| ATOM | 4178 | O | GLU | 256 | 23.866 | 9.040 | 41.018 | 1.00 | 0.00 | −0.396 | 8.17 | −17.40 |
| ATOM | 4179 | CB | GLU | 256 | 24.969 | 11.373 | 42.938 | 1.00 | 0.00 | −0.106 | 12.77 | 4.00 |
| ATOM | 4180 | HB1 | GLU | 256 | 24.924 | 10.952 | 43.942 | 1.00 | 0.00 | 0.053 | 0.00 | 0.00 |
| ATOM | 4181 | HB2 | GLU | 256 | 25.188 | 12.432 | 43.070 | 1.00 | 0.00 | 0.053 | 0.00 | 0.00 |
| ATOM | 4182 | CG | GLU | 256 | 26.216 | 10.769 | 42.290 | 1.00 | 0.00 | −0.106 | 12.77 | 4.00 |
| ATOM | 4183 | HG1 | GLU | 256 | 25.997 | 9.726 | 42.058 | 1.00 | 0.00 | 0.053 | 0.00 | 0.00 |
| ATOM | 4184 | HG2 | GLU | 256 | 27.033 | 10.850 | 43.006 | 1.00 | 0.00 | 0.053 | 0.00 | 0.00 |
| ATOM | 4185 | CD | GLU | 256 | 26.511 | 11.563 | 41.026 | 1.00 | 0.00 | 0.399 | 9.82 | 4.00 |
| ATOM | 4186 | OE1 | GLU | 256 | 25.879 | 12.637 | 40.840 | 1.00 | 0.00 | −0.396 | 8.17 | −18.95 |
| ATOM | 4187 | OE2 | GLU | 256 | 27.371 | 11.103 | 40.227 | 1.00 | 0.00 | −0.427 | 8.17 | −18.95 |
| ATOM | 4189 | N | LYS | 257S | 22.918 | 9.026 | 43.066 | 1.00 | 0.00 | −0.650 | 9.00 | −17.40 |
| ATOM | 4190 | HN | LYS | 257S | 22.598 | 9.563 | 43.884 | 1.00 | 0.00 | 0.440 | 0.00 | 0.00 |
| ATOM | 4191 | CA | LYS | 257S | 22.748 | 7.606 | 43.016 | 1.00 | 0.00 | 0.158 | 9.40 | 4.00 |
| ATOM | 4192 | HA | LYS | 257S | 23.748 | 7.187 | 42.907 | 1.00 | 0.00 | 0.053 | 0.00 | 0.00 |
| ATOM | 4193 | C | LYS | 257S | 21.879 | 7.337 | 41.831 | 1.00 | 0.00 | 0.396 | 9.82 | 4.00 |
| ATOM | 4194 | O | LYS | 257S | 22.182 | 6.451 | 41.026 | 1.00 | 0.00 | −0.396 | 8.17 | −17.40 |
| ATOM | 4195 | CB | LYS | 257S | 22.021 | 7.042 | 44.245 | 1.00 | 0.00 | −0.106 | 12.17 | 4.00 |
| ATOM | 4196 | HB1 | LYS | 257S | 22.003 | 5.955 | 44.162 | 1.00 | 0.00 | 0.053 | 0.00 | 0.00 |
| ATOM | 4197 | HB2 | LYS | 257S | 21.007 | 7.443 | 44.256 | 1.00 | 0.00 | 0.053 | 0.00 | 0.00 |
| ATOM | 4198 | CG | LYS | 257S | 22.675 | 7.395 | 45.583 | 1.00 | 0.00 | −0.106 | 12.77 | 4.00 |
| ATOM | 4199 | HG1 | LYS | 257S | 22.067 | 6.972 | 46.382 | 1.00 | 0.00 | 0.053 | 0.00 | 0.00 |
| ATOM | 4200 | HG2 | LYS | 257S | 22.718 | 8.480 | 45.666 | 1.00 | 0.00 | 0.053 | 0.00 | 0.00 |
| ATOM | 4201 | CD | LYS | 257S | 24.096 | 6.861 | 45.749 | 1.00 | 0.00 | −0.106 | 12.77 | 4.00 |
| ATOM | 4202 | HD1 | LYS | 257S | 24.786 | 7.237 | 44.994 | 1.00 | 0.00 | 0.053 | 0.00 | 0.00 |
| ATOM | 4203 | HD2 | LYS | 257S | 24.156 | 5.774 | 45.684 | 1.00 | 0.00 | 0.053 | 0.00 | 0.00 |
| ATOM | 4204 | CE | LYS | 257S | 24.723 | 7.226 | 47.096 | 1.00 | 0.00 | 0.099 | 12.77 | 4.00 |
| ATOM | 4205 | HE1 | LYS | 257S | 25.685 | 6.725 | 47.203 | 1.00 | 0.00 | 0.053 | 0.00 | 0.00 |
| ATOM | 4206 | HE2 | LYS | 257S | 24.065 | 6.913 | 47.907 | 1.00 | 0.00 | 0.053 | 0.00 | 0.00 |
| ATOM | 4207 | NZ | LYS | 257S | 24.927 | 8.690 | 47.175 | 1.00 | 0.00 | −0.045 | 13.25 | −39.20 |
| ATOM | 4208 | HZ1 | LYS | 257S | 25.349 | 8.929 | 48.083 | 1.00 | 0.00 | 0.280 | 0.00 | 0.00 |
| ATOM | 4209 | HZ2 | LYS | 257S | 25.554 | 8.991 | 46.415 | 1.00 | 0.00 | 0.280 | 0.00 | 0.00 |
| ATOM | 4210 | HZ3 | LYS | 257S | 24.020 | 9.169 | 47.081 | 1.00 | 0.00 | 0.280 | 0.00 | 0.00 |
| ATOM | 4211 | N | ILE | 258 | 20.804 | 8.144 | 41.688 | 1.00 | 0.00 | −0.650 | 9.00 | −17.40 |
| ATOM | 4212 | HN | ILE | 258 | 20.642 | 8.869 | 42.400 | 1.00 | 0.00 | 0.440 | 0.00 | 0.00 |
| ATOM | 4213 | CA | ILE | 258 | 19.874 | 8.045 | 40.596 | 1.00 | 0.00 | 0.158 | 9.40 | 4.00 |
| ATOM | 4214 | HA | ILE | 258 | 18.941 | 8.558 | 40.831 | 1.00 | 0.00 | 0.053 | 0.00 | 0.00 |
| ATOM | 4215 | C | ILE | 258 | 19.553 | 6.618 | 40.298 | 1.00 | 0.00 | 0.396 | 9.82 | 4.00 |
| ATOM | 4216 | O | ILE | 258 | 18.837 | 5.976 | 41.059 | 1.00 | 0.00 | −0.396 | 8.17 | −17.40 |
| ATOM | 4217 | CB | ILE | 258 | 20.349 | 8.771 | 39.362 | 1.00 | 0.00 | −0.053 | 9.40 | 4.00 |
| ATOM | 4218 | HB | ILE | 258 | 20.346 | 9.844 | 39.549 | 1.00 | 0.00 | 0.053 | 0.00 | 0.00 |
| ATOM | 4219 | CG1 | ILE | 258 | 19.310 | 8.638 | 38.236 | 1.00 | 0.00 | −0.106 | 12.77 | 4.00 |
| ATOM | 4220 | HG1 | ILE | 258 | 19.112 | 7.578 | 38.073 | 1.00 | 0.00 | 0.053 | 0.00 | 0.00 |
| ATOM | 4221 | HG1 | ILE | 258 | 19.719 | 9.093 | 37.334 | 1.00 | 0.00 | 0.053 | 0.00 | 0.00 |
| ATOM | 4222 | CG2 | ILE | 258 | 21.773 | 8.314 | 39.017 | 1.00 | 0.00 | −0.159 | 16.15 | 4.00 |
| ATOM | 4223 | HG2 | ILE | 258 | 22.117 | 8.838 | 38.125 | 1.00 | 0.00 | 0.053 | 0.00 | 0.00 |
| ATOM | 4224 | HG2 | ILE | 258 | 22.439 | 8.538 | 39.849 | 1.00 | 0.00 | 0.053 | 0.00 | 0.00 |
| ATOM | 4225 | HG2 | ILE | 258 | 21.775 | 7.240 | 38.830 | 1.00 | 0.00 | 0.053 | 0.00 | 0.00 |
| ATOM | 4226 | CD1 | ILE | 258 | 17.983 | 9.318 | 38.540 | 1.00 | 0.00 | −0.159 | 16.15 | 4.00 |
| ATOM | 4227 | HD1 | ILE | 258 | 17.302 | 9.180 | 37.699 | 1.00 | 0.00 | 0.053 | 0.00 | 0.00 |
| ATOM | 4228 | HD1 | ILE | 258 | 17.545 | 8.878 | 39.436 | 1.00 | 0.00 | 0.053 | 0.00 | 0.00 |
| ATOM | 4229 | HD1 | ILE | 258 | 18.148 | 10.383 | 38.702 | 1.00 | 0.00 | 0.053 | 0.00 | 0.00 |
| ATOM | 4230 | N | GLN | 259 | 20.069 | 6.082 | 39.179 | 1.00 | 0.00 | −0.650 | 9.00 | −17.40 |
| ATOM | 4231 | HN | GLN | 259 | 20.684 | 6.647 | 38.577 | 1.00 | 0.00 | 0.440 | 0.00 | 0.00 |
| ATOM | 4232 | CA | GLN | 259 | 19.766 | 4.725 | 38.818 | 1.00 | 0.00 | 0.158 | 9.40 | 4.00 |
| ATOM | 4233 | HA | GLN | 259 | 18.696 | 4.654 | 38.617 | 1.00 | 0.00 | 0.053 | 0.00 | 0.00 |
| ATOM | 4234 | C | GLN | 259 | 20.147 | 3.839 | 39.961 | 1.00 | 0.00 | 0.396 | 9.82 | 4.00 |
| ATOM | 4235 | O | GLN | 259 | 21.325 | 3.550 | 40.161 | 1.00 | 0.00 | −0.396 | 8.17 | −17.40 |
| ATOM | 4236 | CB | GLN | 259 | 20.523 | 4.232 | 37.573 | 1.00 | 0.00 | −0.106 | 12.77 | 4.00 |
| ATOM | 4237 | HB1 | GLN | 259 | 20.355 | 3.178 | 37.348 | 1.00 | 0.00 | 0.053 | 0.00 | 0.00 |
| ATOM | 4238 | HB2 | GLN | 259 | 21.605 | 4.335 | 37.650 | 1.00 | 0.00 | 0.053 | 0.00 | 0.00 |
| ATOM | 4239 | CG | GLN | 259 | 20.145 | 4.976 | 36.291 | 1.00 | 0.00 | −0.106 | 12.77 | 4.00 |
| ATOM | 4240 | HG1 | GLN | 259 | 19.057 | 5.042 | 36.246 | 1.00 | 0.00 | 0.053 | 0.00 | 0.00 |
| ATOM | 4241 | HG2 | GLN | 259 | 20.533 | 4.409 | 35.444 | 1.00 | 0.00 | 0.053 | 0.00 | 0.00 |
| ATOM | 4242 | CD | GLN | 259 | 20.770 | 6.362 | 36.348 | 1.00 | 0.00 | 0.396 | 9.82 | 4.00 |
| ATOM | 4243 | OE1 | GLN | 259 | 20.162 | 7.351 | 35.942 | 1.00 | 0.00 | −0.396 | 8.17 | −17.40 |
| ATOM | 4244 | NE2 | GLN | 259 | 22.029 | 6.437 | 36.859 | 1.00 | 0.00 | −0.879 | 13.25 | −17.40 |
| ATOM | 4245 | HE2 | GLN | 259 | 22.502 | 5.583 | 37.188 | 1.00 | 0.00 | 0.440 | 0.00 | 0.00 |
| ATOM | 4246 | HE2 | GLN | 259 | 22.508 | 7.347 | 36.916 | 1.00 | 0.00 | 0.440 | 0.00 | 0.00 |
| ATOM | 4247 | N | LYS | 260S | 19.141 | 3.390 | 40.744 | 1.00 | 0.00 | −0.650 | 9.00 | −17.40 |
| ATOM | 4248 | HN | LYS | 260S | 18.168 | 3.642 | 40.517 | 1.00 | 0.00 | 0.440 | 0.00 | 0.00 |
| ATOM | 4249 | CA | LYS | 260S | 19.411 | 2.566 | 41.888 | 1.00 | 0.00 | 0.158 | 9.40 | 4.00 |
| ATOM | 4250 | HA | LYS | 260S | 20.101 | 1.775 | 41.596 | 1.00 | 0.00 | 0.053 | 0.00 | 0.00 |
| ATOM | 4251 | C | LYS | 260S | 18.123 | 1.973 | 42.371 | 1.00 | 0.00 | 0.396 | 9.82 | 4.00 |
| ATOM | 4252 | O | LYS | 260S | 17.552 | 1.084 | 41.742 | 1.00 | 0.00 | −0.396 | 8.17 | −17.40 |
| ATOM | 4253 | CB | LYS | 260S | 19.983 | 3.349 | 43.086 | 1.00 | 0.00 | −0.106 | 12.77 | 4.00 |
| ATOM | 4254 | HB1 | LYS | 260S | 19.995 | 2.686 | 43.951 | 1.00 | 0.00 | 0.053 | 0.00 | 0.00 |
| ATOM | 4255 | HB2 | LYS | 260S | 19.339 | 4.209 | 43.269 | 1.00 | 0.00 | 0.053 | 0.00 | 0.00 |
| ATOM | 4256 | CG | LYS | 260S | 21.407 | 3.871 | 42.882 | 1.00 | 0.00 | −0.106 | 12.77 | 4.00 |
| ATOM | 4257 | HG1 | LYS | 260S | 21.786 | 4.446 | 43.726 | 1.00 | 0.00 | 0.053 | 0.00 | 0.00 |
| ATOM | 4258 | HG2 | LYS | 260S | 21.511 | 4.530 | 42.020 | 1.00 | 0.00 | 0.053 | 0.00 | 0.00 |
| ATOM | 4259 | CD | LYS | 260S | 22.440 | 2.764 | 42.661 | 1.00 | 0.00 | −0.106 | 12.77 | 4.00 |
| ATOM | 4260 | HD1 | LYS | 260S | 22.237 | 2.146 | 41.785 | 1.00 | 0.00 | 0.053 | 0.00 | 0.00 |
| ATOM | 4261 | HD2 | LYS | 260S | 22.512 | 2.061 | 43.491 | 1.00 | 0.00 | 0.053 | 0.00 | 0.00 |
| ATOM | 4262 | CE | LYS | 260S | 23.864 | 3.286 | 42.458 | 1.00 | 0.00 | 0.099 | 12.77 | 4.00 |
| ATOM | 4263 | HE1 | LYS | 260S | 24.183 | 3.849 | 43.334 | 1.00 | 0.00 | 0.053 | 0.00 | 0.00 |
| ATOM | 4264 | HE2 | LYS | 260S | 23.901 | 3.938 | 41.585 | 1.00 | 0.00 | 0.053 | 0.00 | 0.00 |
| ATOM | 4265 | NZ | LYS | 260S | 24.794 | 2.153 | 42.250 | 1.00 | 0.00 | −0.045 | 13.25 | −39.20 |
| ATOM | 4266 | HZ1 | LYS | 260S | 25.749 | 2.512 | 42.114 | 1.00 | 0.00 | 0.280 | 0.00 | 0.00 |
| ATOM | 4267 | HZ2 | LYS | 260S | 24.774 | 1.535 | 43.074 | 1.00 | 0.00 | 0.280 | 0.00 | 0.00 |
| ATOM | 4268 | HZ3 | LYS | 260S | 24.506 | 1.620 | 41.416 | 1.00 | 0.00 | 0.280 | 0.00 | 0.00 |
| ATOM | 4269 | N | LEU | 261 | 17.641 | 2.475 | 43.529 | 1.00 | 0.00 | −0.650 | 9.00 | −17.40 |
| ATOM | 4270 | HN | LEU | 261 | 18.129 | 3.275 | 43.954 | 1.00 | 0.00 | 0.440 | 0.00 | 0.00 |
| ATOM | 4271 | CA | LEU | 261 | 16.483 | 1.950 | 44.197 | 1.00 | 0.00 | 0.158 | 9.40 | 4.00 |
| ATOM | 4272 | HA | LEU | 261 | 16.643 | 0.872 | 44.237 | 1.00 | 0.00 | 0.053 | 0.00 | 0.00 |
| ATOM | 4273 | C | LEU | 261 | 15.317 | 2.352 | 43.332 | 1.00 | 0.00 | 0.396 | 9.82 | 4.00 |
| ATOM | 4274 | O | LEU | 261 | 15.515 | 2.859 | 42.231 | 1.00 | 0.00 | −0.396 | 8.17 | −17.40 |
| ATOM | 4275 | CB | LEU | 261 | 16.357 | 2.542 | 45.620 | 1.00 | 0.00 | −0.106 | 12.77 | 4.00 |
| ATOM | 4276 | HB1 | LEU | 261 | 15.753 | 3.446 | 45.544 | 1.00 | 0.00 | 0.053 | 0.00 | 0.00 |
| ATOM | 4277 | HB2 | LEU | 261 | 17.365 | 2.763 | 45.971 | 1.00 | 0.00 | 0.053 | 0.00 | 0.00 |
| ATOM | 4278 | CG | LEU | 261 | 15.695 | 1.666 | 46.717 | 1.00 | 0.00 | −0.053 | 9.40 | 4.00 |
| ATOM | 4279 | HG | LEU | 261 | 16.334 | 0.817 | 46.960 | 1.00 | 0.00 | 0.053 | 0.00 | 0.00 |
| ATOM | 4280 | CD1 | LEU | 261 | 15.468 | 2.491 | 47.992 | 1.00 | 0.00 | −0.159 | 16.15 | 4.00 |
| ATOM | 4281 | HD1 | LEU | 261 | 15.003 | 1.864 | 48.753 | 1.00 | 0.00 | 0.053 | 0.00 | 0.00 |
| ATOM | 4282 | HD1 | LEU | 261 | 16.424 | 2.860 | 48.361 | 1.00 | 0.00 | 0.053 | 0.00 | 0.00 |
| ATOM | 4283 | HD1 | LEU | 261 | 14.814 | 3.334 | 47.768 | 1.00 | 0.00 | 0.053 | 0.00 | 0.00 |
| ATOM | 4284 | CD2 | LEU | 261 | 14.464 | 0.867 | 46.271 | 1.00 | 0.00 | −0.159 | 16.15 | 4.00 |
| ATOM | 4285 | HD2 | LEU | 261 | 14.080 | 0.290 | 47.112 | 1.00 | 0.00 | 0.053 | 0.00 | 0.00 |
| ATOM | 4286 | HD2 | LEU | 261 | 13.693 | 1.552 | 45.919 | 1.00 | 0.00 | 0.053 | 0.00 | 0.00 |
| ATOM | 4287 | HD2 | LEU | 261 | 14.742 | 0.189 | 45.464 | 1.00 | 0.00 | 0.053 | 0.00 | 0.00 |
| ATOM | 4288 | N | MET | 262 | 14.074 | 2.089 | 43.778 | 1.00 | 0.00 | −0.650 | 9.00 | −17.40 |
| ATOM | 4289 | HN | MET | 262 | 13.973 | 1.694 | 44.724 | 1.00 | 0.00 | 0.440 | 0.00 | 0.00 |
| ATOM | 4290 | CA | MET | 262 | 12.878 | 2.325 | 43.015 | 1.00 | 0.00 | 0.158 | 9.40 | 4.00 |
| ATOM | 4291 | HA | MET | 262 | 13.008 | 1.869 | 42.033 | 1.00 | 0.00 | 0.053 | 0.00 | 0.00 |
| ATOM | 4292 | C | MET | 262 | 12.531 | 3.779 | 42.794 | 1.00 | 0.00 | 0.396 | 9.82 | 4.00 |
| ATOM | 4293 | O | MET | 262 | 12.245 | 4.151 | 41.657 | 1.00 | 0.00 | −0.396 | 8.17 | −17.40 |
| ATOM | 4294 | CB | MET | 262 | 11.640 | 1.662 | 43.644 | 1.00 | 0.00 | −0.106 | 12.77 | 4.00 |
| ATOM | 4295 | HB1 | MET | 262 | 11.388 | 2.053 | 44.629 | 1.00 | 0.00 | 0.053 | 0.00 | 0.00 |
| ATOM | 4296 | HB2 | MET | 262 | 11.744 | 0.586 | 43.785 | 1.00 | 0.00 | 0.053 | 0.00 | 0.00 |
| ATOM | 4297 | CG | MET | 262 | 10.366 | 1.830 | 42.814 | 1.00 | 0.00 | −0.041 | 12.77 | 4.00 |
| ATOM | 4298 | HG1 | MET | 262 | 10.534 | 1.379 | 41.835 | 1.00 | 0.00 | 0.053 | 0.00 | 0.00 |
| ATOM | 4299 | HG2 | MET | 262 | 10.160 | 2.896 | 42.716 | 1.00 | 0.00 | 0.053 | 0.00 | 0.00 |
| ATOM | 4300 | SD | MET | 262 | 8.890 | 1.049 | 43.532 | 1.00 | 0.00 | −0.130 | 16.39 | −6.40 |
| ATOM | 4301 | CE | MET | 262 | 7.776 | 1.548 | 42.187 | 1.00 | 0.00 | −0.094 | 16.15 | 4.00 |
| ATOM | 4302 | HE1 | MET | 262 | 6.770 | 1.180 | 42.391 | 1.00 | 0.00 | 0.053 | 0.00 | 0.00 |
| ATOM | 4303 | HE2 | MET | 262 | 7.756 | 2.635 | 42.115 | 1.00 | 0.00 | 0.053 | 0.00 | 0.00 |
| ATOM | 4304 | HE3 | MET | 262 | 8.131 | 1.128 | 41.245 | 1.00 | 0.00 | 0.053 | 0.00 | 0.00 |
| ATOM | 4305 | N | GLU | 263 | 12.553 | 4.660 | 43.828 | 1.00 | 0.00 | −0.650 | 9.00 | −17.40 |
| ATOM | 4306 | HN | GLU | 263 | 12.937 | 4.406 | 44.749 | 1.00 | 0.00 | 0.440 | 0.00 | 0.00 |
| ATOM | 4307 | CA | GLU | 263 | 12.006 | 5.975 | 43.563 | 1.00 | 0.00 | 0.158 | 9.40 | 4.00 |
| ATOM | 4308 | HA | GLU | 263 | 12.151 | 6.197 | 42.505 | 1.00 | 0.00 | 0.053 | 0.00 | 0.00 |
| ATOM | 4309 | C | GLU | 263 | 12.645 | 7.100 | 44.359 | 1.00 | 0.00 | 0.396 | 9.82 | 4.00 |
| ATOM | 4310 | O | GLU | 263 | 13.776 | 6.997 | 44.828 | 1.00 | 0.00 | −0.396 | 8.17 | −17.40 |
| ATOM | 4311 | CB | GLU | 263 | 10.484 | 6.046 | 43.803 | 1.00 | 0.00 | −0.106 | 12.77 | 4.00 |
| ATOM | 4312 | HB1 | GLU | 263 | 9.999 | 5.349 | 43.119 | 1.00 | 0.00 | 0.053 | 0.00 | 0.00 |
| ATOM | 4313 | HB2 | GLU | 263 | 10.156 | 7.067 | 43.609 | 1.00 | 0.00 | 0.053 | 0.00 | 0.00 |
| ATOM | 4314 | CG | GLU | 263 | 10.040 | 5.682 | 45.220 | 1.00 | 0.00 | −0.106 | 12.77 | 4.00 |
| ATOM | 4315 | HG1 | GLU | 263 | 10.517 | 6.383 | 45.904 | 1.00 | 0.00 | 0.053 | 0.00 | 0.00 |
| ATOM | 4316 | HG2 | GLU | 263 | 10.362 | 4.658 | 45.412 | 1.00 | 0.00 | 0.053 | 0.00 | 0.00 |
| ATOM | 4317 | CD | GLU | 263 | 8.521 | 5.804 | 45.271 | 1.00 | 0.00 | 0.399 | 9.82 | 4.00 |
| ATOM | 4318 | OE1 | GLU | 263 | 7.922 | 6.197 | 44.235 | 1.00 | 0.00 | −0.396 | 8.17 | −18.95 |
| ATOM | 4319 | OE2 | GLU | 263 | 7.939 | 5.500 | 46.347 | 1.00 | 0.00 | −0.427 | 8.17 | −18.95 |
| ATOM | 4321 | N | TRP | 264 | 11.853 | 8.196 | 44.524 | 1.00 | 0.00 | −0.650 | 9.00 | −17.40 |
| ATOM | 4322 | HN | TRP | 264 | 10.899 | 8.041 | 44.167 | 1.00 | 0.00 | 0.440 | 0.00 | 0.00 |
| ATOM | 4323 | CA | TRP | 264 | 12.019 | 9.531 | 45.079 | 1.00 | 0.00 | 0.158 | 9.40 | 4.00 |
| ATOM | 4324 | HA | TRP | 264 | 11.540 | 10.228 | 44.391 | 1.00 | 0.00 | 0.053 | 0.00 | 0.00 |
| ATOM | 4325 | C | TRP | 264 | 13.411 | 10.048 | 45.297 | 1.00 | 0.00 | 0.396 | 9.82 | 4.00 |
| ATOM | 4326 | O | TRP | 264 | 14.302 | 9.366 | 45.800 | 1.00 | 0.00 | −0.396 | 8.17 | −17.40 |
| ATOM | 4327 | CB | TRP | 264 | 11.225 | 9.783 | 46.375 | 1.00 | 0.00 | −0.106 | 12.77 | 4.00 |
| ATOM | 4328 | HB1 | TRP | 264 | 11.425 | 9.049 | 47.156 | 1.00 | 0.00 | 0.053 | 0.00 | 0.00 |
| ATOM | 4329 | HB2 | TRP | 264 | 10.144 | 9.761 | 46.232 | 1.00 | 0.00 | 0.053 | 0.00 | 0.00 |
| ATOM | 4330 | CG | TRP | 264 | 11.516 | 11.127 | 47.008 | 1.00 | 0.00 | 0.000 | 7.26 | 0.60 |
| ATOM | 4331 | CD1 | TRP | 264 | 12.467 | 11.429 | 47.936 | 1.00 | 0.00 | −0.177 | 10.80 | 0.60 |
| ATOM | 4332 | HD1 | TRP | 264 | 13.171 | 10.714 | 48.361 | 1.00 | 0.00 | 0.127 | 0.00 | 0.00 |
| ATOM | 4333 | CD2 | TRP | 264 | 10.822 | 12.356 | 46.734 | 1.00 | 0.00 | 0.000 | 6.80 | 0.60 |
| ATOM | 4334 | NE1 | TRP | 264 | 12.412 | 12.761 | 48.258 | 1.00 | 0.00 | −0.292 | 9.00 | −17.40 |
| ATOM | 4335 | HE1 | TRP | 264 | 13.022 | 13.242 | 48.933 | 1.00 | 0.00 | 0.393 | 0.00 | 0.00 |
| ATOM | 4336 | CE2 | TRP | 264 | 11.404 | 13.346 | 47.527 | 1.00 | 0.00 | −0.050 | 6.80 | 0.60 |
| ATOM | 4337 | CE3 | TRP | 264 | 9.786 | 12.639 | 45.896 | 1.00 | 0.00 | −0.127 | 10.80 | 0.60 |
| ATOM | 4338 | HE3 | TRP | 264 | 9.329 | 11.864 | 45.279 | 1.00 | 0.00 | 0.127 | 0.00 | 0.00 |
| ATOM | 4339 | CZ2 | TRP | 264 | 10.957 | 14.635 | 47.491 | 1.00 | 0.00 | −0.127 | 10.80 | 0.60 |
| ATOM | 4340 | HZ2 | TRP | 264 | 11.409 | 15.409 | 48.110 | 1.00 | 0.00 | 0.127 | 0.00 | 0.00 |
| ATOM | 4341 | CZ3 | TRP | 264 | 9.338 | 13.940 | 45.857 | 1.00 | 0.00 | −0.127 | 10.80 | 0.60 |
| ATOM | 4342 | HZ3 | TRP | 264 | 8.513 | 14.202 | 45.194 | 1.00 | 0.00 | 0.127 | 0.00 | 0.00 |
| ATOM | 4343 | CH2 | TRP | 264 | 9.913 | 14.918 | 46.641 | 1.00 | 0.00 | −0.127 | 10.80 | 0.60 |
| ATOM | 4344 | HH2 | TRP | 264 | 9.531 | 15.937 | 46.586 | 1.00 | 0.00 | 0.127 | 0.00 | 0.00 |
| ATOM | 4345 | N | ALA | 265 | 13.590 | 11.329 | 44.871 | 1.00 | 0.00 | −0.650 | 9.00 | −17.40 |
| ATOM | 4346 | HN | ALA | 265 | 12.792 | 11.765 | 44.387 | 1.00 | 0.00 | 0.440 | 0.00 | 0.00 |
| ATOM | 4347 | CA | ALA | 265 | 14.770 | 12.142 | 45.021 | 1.00 | 0.00 | 0.158 | 9.40 | 4.00 |
| ATOM | 4348 | HA | ALA | 265 | 15.307 | 11.762 | 45.889 | 1.00 | 0.00 | 0.053 | 0.00 | 0.00 |
| ATOM | 4349 | C | ALA | 265 | 14.303 | 13.557 | 45.224 | 1.00 | 0.00 | 0.396 | 9.82 | 4.00 |
| ATOM | 4350 | O | ALA | 265 | 13.202 | 13.933 | 44.818 | 1.00 | 0.00 | −0.396 | 8.17 | −17.40 |
| ATOM | 4351 | CB | ALA | 265 | 15.731 | 12.132 | 43.818 | 1.00 | 0.00 | −0.159 | 16.15 | 4.00 |
| ATOM | 4352 | HB1 | ALA | 265 | 16.586 | 12.773 | 44.030 | 1.00 | 0.00 | 0.053 | 0.00 | 0.00 |
| ATOM | 4353 | HB2 | ALA | 265 | 16.077 | 11.114 | 43.637 | 1.00 | 0.00 | 0.053 | 0.00 | 0.00 |
| ATOM | 4354 | HB3 | ALA | 265 | 15.211 | 12.500 | 42.933 | 1.00 | 0.00 | 0.053 | 0.00 | 0.00 |
| ATOM | 4355 | N | GLN | 266 | 15.160 | 14.391 | 45.851 | 1.00 | 0.00 | −0.650 | 9.00 | −17.40 |
| ATOM | 4356 | HN | GLN | 266 | 16.104 | 14.045 | 46.073 | 1.00 | 0.00 | 0.440 | 0.00 | 0.00 |
| ATOM | 4357 | CA | GLN | 266 | 14.820 | 15.737 | 46.224 | 1.00 | 0.00 | 0.158 | 9.40 | 4.00 |
| ATOM | 4358 | HA | GLN | 266 | 13.943 | 15.756 | 46.871 | 1.00 | 0.00 | 0.053 | 0.00 | 0.00 |
| ATOM | 4359 | C | GLN | 266 | 14.516 | 16.569 | 45.021 | 1.00 | 0.00 | 0.396 | 9.82 | 4.00 |
| ATOM | 4360 | O | GLN | 266 | 14.378 | 16.069 | 43.906 | 1.00 | 0.00 | −0.396 | 8.17 | −17.40 |
| ATOM | 4361 | CB | GLN | 266 | 15.940 | 16.449 | 47.001 | 1.00 | 0.00 | −0.106 | 12.77 | 4.00 |
| ATOM | 4362 | HB1 | GLN | 266 | 15.715 | 17.490 | 47.231 | 1.00 | 0.00 | 0.053 | 0.00 | 0.00 |
| ATOM | 4363 | HB2 | GLN | 266 | 16.890 | 16.474 | 46.468 | 1.00 | 0.00 | 0.053 | 0.00 | 0.00 |
| ATOM | 4364 | CG | GLN | 266 | 16.255 | 15.799 | 48.352 | 1.00 | 0.00 | −0.106 | 12.77 | 4.00 |
| ATOM | 4365 | HG1 | GLN | 266 | 16.567 | 14.771 | 48.164 | 1.00 | 0.00 | 0.053 | 0.00 | 0.00 |
| ATOM | 4366 | HG2 | GLN | 266 | 15.348 | 15.827 | 48.956 | 1.00 | 0.00 | 0.053 | 0.00 | 0.00 |
| ATOM | 4367 | CD | GLN | 266 | 17.372 | 16.599 | 49.006 | 1.00 | 0.00 | 0.396 | 9.82 | 4.00 |
| ATOM | 4368 | OE1 | GLN | 266 | 17.852 | 17.587 | 48.452 | 1.00 | 0.00 | −0.396 | 8.17 | −17.40 |
| ATOM | 4369 | NE2 | GLN | 266 | 17.801 | 16.160 | 50.219 | 1.00 | 0.00 | −0.879 | 13.25 | −17.40 |
| ATOM | 4370 | HE2 | GLN | 266 | 17.370 | 15.327 | 50.647 | 1.00 | 0.00 | 0.440 | 0.00 | 0.00 |
| ATOM | 4371 | HE2 | GLN | 266 | 18.556 | 16.659 | 50.709 | 1.00 | 0.00 | 0.440 | 0.00 | 0.00 |
| ATOM | 4372 | N | ASP | 267P | 14.395 | 17.892 | 45.262 | 1.00 | 0.00 | −0.650 | 9.00 | −17.40 |
| ATOM | 4373 | HN | ASP | 267P | 14.562 | 18.221 | 46.223 | 1.00 | 0.00 | 0.440 | 0.00 | 0.00 |
| ATOM | 4374 | CA | ASP | 267P | 14.049 | 18.870 | 44.270 | 1.00 | 0.00 | 0.158 | 9.40 | 4.00 |
| ATOM | 4375 | HA | ASP | 267P | 13.175 | 18.514 | 43.724 | 1.00 | 0.00 | 0.053 | 0.00 | 0.00 |
| ATOM | 4376 | C | ASP | 267P | 15.207 | 19.037 | 43.347 | 1.00 | 0.00 | 0.396 | 9.82 | 4.00 |
| ATOM | 4377 | O | ASP | 267P | 15.576 | 18.096 | 42.648 | 1.00 | 0.00 | −0.396 | 8.17 | −17.40 |
| ATOM | 4378 | CB | ASP | 267P | 13.726 | 20.249 | 44.870 | 1.00 | 0.00 | −0.336 | 12.77 | 4.00 |
| ATOM | 4379 | HB1 | ASP | 267P | 14.649 | 20.678 | 45.259 | 1.00 | 0.00 | 0.053 | 0.00 | 0.00 |
| ATOM | 4380 | HB2 | ASP | 267P | 12.999 | 20.112 | 45.670 | 1.00 | 0.00 | 0.053 | 0.00 | 0.00 |
| ATOM | 4381 | CG | ASP | 267P | 13.150 | 21.128 | 43.768 | 1.00 | 0.00 | 0.297 | 9.82 | 4.00 |
| ATOM | 4382 | OD1 | ASP | 267P | 13.137 | 20.676 | 42.592 | 1.00 | 0.00 | −0.534 | 8.17 | −18.95 |
| ATOM | 4383 | OD2 | ASP | 267P | 12.709 | 22.264 | 44.089 | 1.00 | 0.00 | −0.534 | 8.17 | −18.95 |
| ATOM | 4384 | N | THR | 268 | 15.806 | 20.249 | 43.352 | 1.00 | 0.00 | −0.650 | 9.00 | −17.40 |
| ATOM | 4385 | HN | THR | 268 | 15.461 | 20.948 | 44.024 | 1.00 | 0.00 | 0.440 | 0.00 | 0.00 |
| ATOM | 4386 | CA | THR | 268 | 16.887 | 20.632 | 42.488 | 1.00 | 0.00 | 0.158 | 9.40 | 4.00 |
| ATOM | 4387 | HA | THR | 268 | 17.009 | 21.714 | 42.524 | 1.00 | 0.00 | 0.053 | 0.00 | 0.00 |
| ATOM | 4388 | C | THR | 268 | 16.573 | 20.206 | 41.085 | 1.00 | 0.00 | 0.396 | 9.82 | 4.00 |
| ATOM | 4389 | O | THR | 268 | 16.790 | 19.071 | 40.666 | 1.00 | 0.00 | −0.396 | 8.17 | −17.40 |
| ATOM | 4390 | CB | THR | 268 | 18.231 | 20.129 | 42.949 | 1.00 | 0.00 | 0.060 | 9.40 | 4.00 |
| ATOM | 4391 | HB | THR | 268 | 18.433 | 20.470 | 43.964 | 1.00 | 0.00 | 0.053 | 0.00 | 0.00 |
| ATOM | 4392 | OG1 | THR | 268 | 19.264 | 20.670 | 42.139 | 1.00 | 0.00 | −0.537 | 11.04 | −17.40 |
| ATOM | 4393 | HG1 | THR | 268 | 19.884 | 21.254 | 42.717 | 1.00 | 0.00 | 0.424 | 0.00 | 0.00 |
| ATOM | 4394 | CG2 | THR | 268 | 18.258 | 18.592 | 42.931 | 1.00 | 0.00 | −0.159 | 16.15 | 4.00 |
| ATOM | 4395 | HG2 | THR | 268 | 19.234 | 18.241 | 43.266 | 1.00 | 0.00 | 0.053 | 0.00 | 0.00 |
| ATOM | 4396 | HG2 | THR | 268 | 17.485 | 18.206 | 43.596 | 1.00 | 0.00 | 0.053 | 0.00 | 0.00 |
| ATOM | 4397 | HG2 | THR | 268 | 18.073 | 18.236 | 41.917 | 1.00 | 0.00 | 0.053 | 0.00 | 0.00 |
| ATOM | 4398 | N | TYR | 269 | 16.037 | 21.165 | 40.311 | 1.00 | 0.00 | −0.650 | 9.00 | −17.40 |
| ATOM | 4399 | HN | TYR | 269 | 15.958 | 22.116 | 40.697 | 1.00 | 0.00 | 0.440 | 0.00 | 0.00 |
| ATOM | 4400 | CA | TYR | 269 | 15.570 | 20.934 | 38.978 | 1.00 | 0.00 | 0.158 | 9.40 | 4.00 |
| ATOM | 4401 | HA | TYR | 269 | 14.839 | 20.125 | 38.965 | 1.00 | 0.00 | 0.053 | 0.00 | 0.00 |
| ATOM | 4402 | C | TYR | 269 | 16.709 | 20.559 | 38.083 | 1.00 | 0.00 | 0.396 | 9.82 | 4.00 |
| ATOM | 4403 | O | TYR | 269 | 16.531 | 19.774 | 37.154 | 1.00 | 0.00 | −0.396 | 8.17 | −17.40 |
| ATOM | 4404 | CB | TYR | 269 | 14.888 | 22.172 | 38.374 | 1.00 | 0.00 | −0.106 | 12.77 | 4.00 |
| ATOM | 4405 | HB1 | TYR | 269 | 15.655 | 22.934 | 38.242 | 1.00 | 0.00 | 0.053 | 0.00 | 0.00 |
| ATOM | 4406 | HB2 | TYR | 269 | 14.119 | 22.495 | 39.076 | 1.00 | 0.00 | 0.053 | 0.00 | 0.00 |
| ATOM | 4407 | CG | TYR | 269 | 14.297 | 21.762 | 37.070 | 1.00 | 0.00 | 0.000 | 7.26 | 0.60 |
| ATOM | 4408 | CD1 | TYR | 269 | 13.083 | 21.115 | 37.042 | 1.00 | 0.00 | −0.127 | 10.80 | 0.60 |
| ATOM | 4409 | HD1 | TYR | 269 | 12.562 | 20.905 | 37.976 | 1.00 | 0.00 | 0.127 | 0.00 | 0.00 |
| ATOM | 4410 | CD2 | TYR | 269 | 14.945 | 22.022 | 35.885 | 1.00 | 0.00 | −0.127 | 10.80 | 0.60 |
| ATOM | 4411 | HD2 | TYR | 269 | 15.906 | 22.534 | 35.894 | 1.00 | 0.00 | 0.127 | 0.00 | 0.00 |
| ATOM | 4412 | CE1 | TYR | 269 | 12.520 | 20.730 | 35.850 | 1.00 | 0.00 | −0.127 | 10.80 | 0.60 |
| ATOM | 4413 | HE1 | TYR | 269 | 11.557 | 20.217 | 35.839 | 1.00 | 0.00 | 0.127 | 0.00 | 0.00 |
| ATOM | 4414 | CE2 | TYR | 269 | 14.387 | 21.639 | 34.688 | 1.00 | 0.00 | −0.127 | 10.80 | 0.60 |
| ATOM | 4415 | HE2 | TYR | 269 | 14.906 | 21.847 | 33.752 | 1.00 | 0.00 | 0.127 | 0.00 | 0.00 |
| ATOM | 4416 | CZ | TYR | 269 | 13.174 | 20.993 | 34.671 | 1.00 | 0.00 | 0.026 | 7.26 | 0.60 |
| ATOM | 4417 | OH | TYR | 269 | 12.598 | 20.599 | 33.446 | 1.00 | 0.00 | −0.451 | 10.94 | −17.40 |
| ATOM | 4418 | HH | TYR | 269 | 12.186 | 21.420 | 32.979 | 1.00 | 0.00 | 0.424 | 0.00 | 0.00 |
| ATOM | 4419 | N | SER | 270 | 17.919 | 21.094 | 38.338 | 1.00 | 0.00 | −0.650 | 9.00 | −17.40 |
| ATOM | 4420 | HN | SER | 270 | 18.058 | 21.684 | 39.170 | 1.00 | 0.00 | 0.440 | 0.00 | 0.00 |
| ATOM | 4421 | CA | SER | 270 | 19.012 | 20.837 | 37.444 | 1.00 | 0.00 | 0.158 | 9.40 | 4.00 |
| ATOM | 4422 | HA | SER | 270 | 18.777 | 21.176 | 36.435 | 1.00 | 0.00 | 0.053 | 0.00 | 0.00 |
| ATOM | 4423 | C | SER | 270 | 19.317 | 19.369 | 37.380 | 1.00 | 0.00 | 0.396 | 9.82 | 4.00 |
| ATOM | 4424 | O | SER | 270 | 19.726 | 18.887 | 36.325 | 1.00 | 0.00 | −0.396 | 8.17 | −17.40 |
| ATOM | 4425 | CB | SER | 270 | 20.286 | 21.674 | 37.721 | 1.00 | 0.00 | 0.007 | 12.77 | 4.00 |
| ATOM | 4426 | HB1 | SER | 270 | 20.119 | 22.745 | 37.614 | 1.00 | 0.00 | 0.053 | 0.00 | 0.00 |
| ATOM | 4427 | HB2 | SER | 270 | 21.106 | 21.430 | 37.046 | 1.00 | 0.00 | 0.053 | 0.00 | 0.00 |
| ATOM | 4428 | OG | SER | 270 | 20.792 | 21.488 | 39.033 | 1.00 | 0.00 | −0.537 | 11.04 | −17.40 |
| ATOM | 4429 | HG | SER | 270 | 20.787 | 22.387 | 39.534 | 1.00 | 0.00 | 0.424 | 0.00 | 0.00 |
| ATOM | 4430 | N | PRO | 271 | 19.141 | 18.612 | 38.427 | 1.00 | 0.00 | −0.422 | 9.00 | −17.40 |
| ATOM | 4431 | CA | PRO | 271 | 19.399 | 17.207 | 38.302 | 1.00 | 0.00 | 0.158 | 9.40 | 4.00 |
| ATOM | 4432 | HA | PRO | 271 | 20.388 | 17.031 | 37.879 | 1.00 | 0.00 | 0.053 | 0.00 | 0.00 |
| ATOM | 4433 | CD | PRO | 271 | 19.458 | 19.081 | 39.765 | 1.00 | 0.00 | 0.105 | 12.77 | 4.00 |
| ATOM | 4434 | HD1 | PRO | 271 | 18.529 | 19.483 | 40.170 | 1.00 | 0.00 | 0.053 | 0.00 | 0.00 |
| ATOM | 4435 | HD2 | PRO | 271 | 20.228 | 19.842 | 39.641 | 1.00 | 0.00 | 0.053 | 0.00 | 0.00 |
| ATOM | 4436 | C | PRO | 271 | 18.395 | 16.563 | 37.412 | 1.00 | 0.00 | 0.396 | 9.82 | 4.00 |
| ATOM | 4437 | O | PRO | 271 | 18.560 | 15.389 | 37.094 | 1.00 | 0.00 | −0.396 | 8.17 | −17.40 |
| ATOM | 4438 | CB | PRO | 271 | 19.445 | 16.654 | 39.722 | 1.00 | 0.00 | −0.106 | 12.77 | 4.00 |
| ATOM | 4439 | HB1 | PRO | 271 | 20.115 | 15.796 | 39.788 | 1.00 | 0.00 | 0.053 | 0.00 | 0.00 |
| ATOM | 4440 | HB2 | PRO | 271 | 18.457 | 16.329 | 40.050 | 1.00 | 0.00 | 0.053 | 0.00 | 0.00 |
| ATOM | 4441 | CG | PRO | 271 | 19.958 | 17.850 | 40.538 | 1.00 | 0.00 | −0.106 | 12.77 | 4.00 |
| ATOM | 4442 | HG1 | PRO | 271 | 21.041 | 17.733 | 40.535 | 1.00 | 0.00 | 0.053 | 0.00 | 0.00 |
| ATOM | 4443 | HG2 | PRO | 271 | 19.499 | 17.724 | 41.518 | 1.00 | 0.00 | 0.053 | 0.00 | 0.00 |
| ATOM | 4444 | N | ASP | 272P | 17.339 | 17.286 | 37.005 | 1.00 | 0.00 | −0.650 | 9.00 | −17.40 |
| ATOM | 4445 | HN | ASP | 272P | 17.218 | 18.262 | 37.309 | 1.00 | 0.00 | 0.440 | 0.00 | 0.00 |
| ATOM | 4446 | CA | ASP | 272P | 16.386 | 16.666 | 36.138 | 1.00 | 0.00 | 0.158 | 9.40 | 4.00 |
| ATOM | 4447 | HA | ASP | 272P | 16.007 | 15.798 | 36.678 | 1.00 | 0.00 | 0.053 | 0.00 | 0.00 |
| ATOM | 4448 | C | ASP | 272P | 17.136 | 16.293 | 34.901 | 1.00 | 0.00 | 0.396 | 9.82 | 4.00 |
| ATOM | 4449 | O | ASP | 272P | 16.969 | 15.201 | 34.358 | 1.00 | 0.00 | −0.396 | 8.17 | −17.40 |
| ATOM | 4450 | CB | ASP | 272P | 15.230 | 17.608 | 35.756 | 1.00 | 0.00 | −0.336 | 12.77 | 4.00 |
| ATOM | 4451 | HB1 | ASP | 272P | 15.588 | 18.283 | 34.979 | 1.00 | 0.00 | 0.053 | 0.00 | 0.00 |
| ATOM | 4452 | HB2 | ASP | 272P | 14.937 | 18.161 | 36.648 | 1.00 | 0.00 | 0.053 | 0.00 | 0.00 |
| ATOM | 4453 | CG | ASP | 272P | 14.078 | 16.755 | 35.244 | 1.00 | 0.00 | 0.297 | 9.82 | 4.00 |
| ATOM | 4454 | OD1 | ASP | 272P | 14.269 | 15.516 | 35.120 | 1.00 | 0.00 | −0.534 | 8.17 | −18.95 |
| ATOM | 4455 | OD2 | ASP | 272P | 12.991 | 17.332 | 34.970 | 1.00 | 0.00 | −0.534 | 8.17 | −18.95 |
| ATOM | 4456 | N | GLU | 273 | 18.003 | 17.206 | 34.430 | 1.00 | 0.00 | −0.650 | 9.00 | −17.40 |
| ATOM | 4457 | HN | GLU | 273 | 18.096 | 18.107 | 34.920 | 1.00 | 0.00 | 0.440 | 0.00 | 0.00 |
| ATOM | 4458 | CA | GLU | 273 | 18.801 | 16.961 | 33.266 | 1.00 | 0.00 | 0.158 | 9.40 | 4.00 |
| ATOM | 4459 | HA | GLU | 273 | 18.132 | 16.671 | 32.455 | 1.00 | 0.00 | 0.053 | 0.00 | 0.00 |
| ATOM | 4460 | C | GLU | 273 | 19.760 | 15.858 | 33.588 | 1.00 | 0.00 | 0.396 | 9.82 | 4.00 |
| ATOM | 4461 | O | GLU | 273 | 20.070 | 15.019 | 32.745 | 1.00 | 0.00 | −0.396 | 8.17 | −17.40 |
| ATOM | 4462 | CB | GLU | 273 | 19.619 | 18.192 | 32.836 | 1.00 | 0.00 | −0.106 | 12.77 | 4.00 |
| ATOM | 4463 | HB1 | GLU | 273 | 20.350 | 18.408 | 33.614 | 1.00 | 0.00 | 0.053 | 0.00 | 0.00 |
| ATOM | 4464 | HB2 | GLU | 273 | 18.936 | 19.032 | 32.709 | 1.00 | 0.00 | 0.053 | 0.00 | 0.00 |
| ATOM | 4465 | CG | GLU | 273 | 20.377 | 18.002 | 31.520 | 1.00 | 0.00 | −0.106 | 12.77 | 4.00 |
| ATOM | 4466 | HG1 | GLU | 273 | 20.604 | 18.983 | 31.103 | 1.00 | 0.00 | 0.053 | 0.00 | 0.00 |
| ATOM | 4467 | HG2 | GLU | 273 | 19.747 | 17.436 | 30.832 | 1.00 | 0.00 | 0.053 | 0.00 | 0.00 |
| ATOM | 4468 | CD | GLU | 273 | 21.663 | 17.240 | 31.804 | 1.00 | 0.00 | 0.399 | 9.82 | 4.00 |
| ATOM | 4469 | OE1 | GLU | 273 | 22.080 | 17.197 | 32.992 | 1.00 | 0.00 | −0.396 | 8.17 | −18.95 |
| ATOM | 4470 | OE2 | GLU | 273 | 22.251 | 16.694 | 30.832 | 1.00 | 0.00 | −0.427 | 8.17 | −18.95 |
| ATOM | 4472 | N | TYR | 274 | 20.268 | 15.840 | 34.831 | 1.00 | 0.00 | −0.650 | 9.00 | −17.40 |
| ATOM | 4473 | HN | TYR | 274 | 19.960 | 16.546 | 35.514 | 1.00 | 0.00 | 0.440 | 0.00 | 0.00 |
| ATOM | 4474 | CA | TYR | 274 | 21.231 | 14.854 | 35.221 | 1.00 | 0.00 | 0.158 | 9.40 | 4.00 |
| ATOM | 4475 | HA | TYR | 274 | 22.078 | 14.880 | 34.535 | 1.00 | 0.00 | 0.053 | 0.00 | 0.00 |
| ATOM | 4476 | C | TYR | 274 | 20.603 | 13.498 | 35.182 | 1.00 | 0.00 | 0.396 | 9.82 | 4.00 |
| ATOM | 4477 | O | TYR | 274 | 21.236 | 12.527 | 34.773 | 1.00 | 0.00 | −0.396 | 8.17 | −17.40 |
| ATOM | 4478 | CB | TYR | 274 | 21.769 | 15.078 | 36.646 | 1.00 | 0.00 | −0.106 | 12.77 | 4.00 |
| ATOM | 4479 | HB1 | TYR | 274 | 20.926 | 15.027 | 37.335 | 1.00 | 0.00 | 0.053 | 0.00 | 0.00 |
| ATOM | 4480 | HB2 | TYR | 274 | 22.239 | 16.060 | 36.677 | 1.00 | 0.00 | 0.053 | 0.00 | 0.00 |
| ATOM | 4481 | CG | TYR | 274 | 22.761 | 14.003 | 36.938 | 1.00 | 0.00 | 0.000 | 7.26 | 0.60 |
| ATOM | 4482 | CD1 | TYR | 274 | 22.356 | 12.791 | 37.445 | 1.00 | 0.00 | −0.127 | 10.80 | 0.60 |
| ATOM | 4483 | HD1 | TYR | 274 | 21.297 | 12.613 | 37.636 | 1.00 | 0.00 | 0.127 | 0.00 | 0.00 |
| ATOM | 4484 | CD2 | TYR | 274 | 24.102 | 14.210 | 36.701 | 1.00 | 0.00 | −0.127 | 10.80 | 0.60 |
| ATOM | 4485 | HD2 | TYR | 274 | 24.436 | 15.165 | 36.297 | 1.00 | 0.00 | 0.127 | 0.00 | 0.00 |
| ATOM | 4486 | CE1 | TYR | 274 | 23.273 | 11.800 | 37.713 | 1.00 | 0.00 | −0.127 | 10.80 | 0.60 |
| ATOM | 4487 | HE1 | TYR | 274 | 22.939 | 10.842 | 38.114 | 1.00 | 0.00 | 0.127 | 0.00 | 0.00 |
| ATOM | 4488 | CE2 | TYR | 274 | 25.022 | 13.226 | 36.967 | 1.00 | 0.00 | −0.127 | 10.80 | 0.60 |
| ATOM | 4489 | HE2 | TYR | 274 | 26.080 | 13.403 | 36.775 | 1.00 | 0.00 | 0.127 | 0.00 | 0.00 |
| ATOM | 4490 | CZ | TYR | 274 | 24.609 | 12.018 | 37.474 | 1.00 | 0.00 | 0.026 | 7.26 | 0.60 |
| ATOM | 4491 | OH | TYR | 274 | 25.552 | 11.005 | 37.748 | 1.00 | 0.00 | −0.451 | 10.94 | −17.40 |
| ATOM | 4492 | HH | TYR | 274 | 25.084 | 10.219 | 38.223 | 1.00 | 0.00 | 0.424 | 0.00 | 0.00 |
| ATOM | 4493 | N | LEU | 275 | 19.326 | 13.402 | 35.583 | 1.00 | 0.00 | −0.650 | 9.00 | −17.40 |
| ATOM | 4494 | HN | LEU | 275 | 18.801 | 14.257 | 35.817 | 1.00 | 0.00 | 0.440 | 0.00 | 0.00 |
| ATOM | 4495 | CA | LEU | 275 | 18.682 | 12.128 | 35.690 | 1.00 | 0.00 | 0.158 | 9.40 | 4.00 |
| ATOM | 4496 | HA | LEU | 275 | 19.223 | 11.542 | 36.432 | 1.00 | 0.00 | 0.053 | 0.00 | 0.00 |
| ATOM | 4497 | C | LEU | 275 | 18.735 | 11.480 | 34.356 | 1.00 | 0.00 | 0.396 | 9.82 | 4.00 |
| ATOM | 4498 | O | LEU | 275 | 19.129 | 10.324 | 34.223 | 1.00 | 0.00 | −0.396 | 8.17 | −17.40 |
| ATOM | 4499 | CB | LEU | 275 | 17.202 | 12.244 | 36.073 | 1.00 | 0.00 | −0.106 | 12.77 | 4.00 |
| ATOM | 4500 | HB1 | LEU | 275 | 16.701 | 11.278 | 36.147 | 1.00 | 0.00 | 0.053 | 0.00 | 0.00 |
| ATOM | 4501 | HB2 | LEU | 275 | 16.617 | 12.821 | 35.356 | 1.00 | 0.00 | 0.053 | 0.00 | 0.00 |
| ATOM | 4502 | CG | LEU | 275 | 16.980 | 12.930 | 37.434 | 1.00 | 0.00 | −0.053 | 9.40 | 4.00 |
| ATOM | 4503 | HG | LEU | 275 | 17.381 | 13.943 | 37.433 | 1.00 | 0.00 | 0.053 | 0.00 | 0.00 |
| ATOM | 4504 | CD1 | LEU | 275 | 15.485 | 13.019 | 37.772 | 1.00 | 0.00 | −0.159 | 16.15 | 4.00 |
| ATOM | 4505 | HD1 | LEU | 275 | 15.358 | 13.507 | 38.738 | 1.00 | 0.00 | 0.053 | 0.00 | 0.00 |
| ATOM | 4506 | HD1 | LEU | 275 | 14.972 | 13.596 | 37.003 | 1.00 | 0.00 | 0.053 | 0.00 | 0.00 |
| ATOM | 4507 | HD1 | LEU | 275 | 15.061 | 12.015 | 37.814 | 1.00 | 0.00 | 0.053 | 0.00 | 0.00 |
| ATOM | 4508 | CD2 | LEU | 275 | 17.812 | 12.264 | 38.541 | 1.00 | 0.00 | −0.159 | 16.15 | 4.00 |
| ATOM | 4509 | HD2 | LEU | 275 | 17.631 | 12.772 | 39.487 | 1.00 | 0.00 | 0.053 | 0.00 | 0.00 |
| ATOM | 4510 | HD2 | LEU | 275 | 17.524 | 11.216 | 38.631 | 1.00 | 0.00 | 0.053 | 0.00 | 0.00 |
| ATOM | 4511 | HD2 | LEU | 275 | 18.870 | 12.329 | 38.289 | 1.00 | 0.00 | 0.053 | 0.00 | 0.00 |
| ATOM | 4512 | N | TRP | 276 | 18.339 | 12.224 | 33.316 | 1.00 | 0.00 | −0.650 | 9.00 | −17.40 |
| ATOM | 4513 | HN | TRP | 276 | 17.985 | 13.179 | 33.463 | 1.00 | 0.00 | 0.440 | 0.00 | 0.00 |
| ATOM | 4514 | CA | TRP | 276 | 18.413 | 11.678 | 32.003 | 1.00 | 0.00 | 0.158 | 9.40 | 4.00 |
| ATOM | 4515 | HA | TRP | 276 | 19.446 | 11.344 | 31.905 | 1.00 | 0.00 | 0.053 | 0.00 | 0.00 |
| ATOM | 4516 | C | TRP | 276 | 18.058 | 12.806 | 31.114 | 1.00 | 0.00 | 0.396 | 9.82 | 4.00 |
| ATOM | 4517 | O | TRP | 276 | 18.126 | 13.964 | 31.518 | 1.00 | 0.00 | −0.396 | 8.17 | −17.40 |
| ATOM | 4518 | CB | TRP | 276 | 17.444 | 10.504 | 31.750 | 1.00 | 0.00 | −0.106 | 12.77 | 4.00 |
| ATOM | 4519 | HB1 | TRP | 276 | 17.604 | 9.765 | 32.535 | 1.00 | 0.00 | 0.053 | 0.00 | 0.00 |
| ATOM | 4520 | HB2 | TRP | 276 | 17.672 | 10.089 | 30.768 | 1.00 | 0.00 | 0.053 | 0.00 | 0.00 |
| ATOM | 4521 | CG | TRP | 276 | 15.970 | 10.836 | 31.754 | 1.00 | 0.00 | 0.000 | 7.26 | 0.60 |
| ATOM | 4522 | CD1 | TRP | 276 | 15.122 | 11.018 | 32.807 | 1.00 | 0.00 | −0.177 | 10.80 | 0.60 |
| ATOM | 4523 | HD1 | TRP | 276 | 15.412 | 10.981 | 33.856 | 1.00 | 0.00 | 0.127 | 0.00 | 0.00 |
| ATOM | 4524 | CD2 | TRP | 276 | 15.169 | 10.951 | 30.565 | 1.00 | 0.00 | 0.000 | 6.80 | 0.60 |
| ATOM | 4525 | NE1 | TRP | 276 | 13.847 | 11.254 | 32.350 | 1.00 | 0.00 | −0.292 | 9.00 | −17.40 |
| ATOM | 4526 | HE1 | TRP | 276 | 13.020 | 11.433 | 32.938 | 1.00 | 0.00 | 0.393 | 0.00 | 0.00 |
| ATOM | 4527 | CE2 | TRP | 276 | 13.861 | 11.210 | 30.972 | 1.00 | 0.00 | −0.050 | 6.80 | 0.60 |
| ATOM | 4528 | CE3 | TRP | 276 | 15.495 | 10.836 | 29.244 | 1.00 | 0.00 | −0.127 | 10.80 | 0.60 |
| ATOM | 4529 | HE3 | TRP | 276 | 16.516 | 10.621 | 28.929 | 1.00 | 0.00 | 0.127 | 0.00 | 0.00 |
| ATOM | 4530 | CZ2 | TRP | 276 | 12.856 | 11.362 | 30.059 | 1.00 | 0.00 | −0.127 | 10.80 | 0.60 |
| ATOM | 4531 | HZ2 | TRP | 276 | 11.830 | 11.556 | 30.372 | 1.00 | 0.00 | 0.127 | 0.00 | 0.00 |
| ATOM | 4532 | CZ3 | TRP | 276 | 14.484 | 11.001 | 28.324 | 1.00 | 0.00 | −0.127 | 10.80 | 0.60 |
| ATOM | 4533 | HZ3 | TRP | 276 | 14.709 | 10.925 | 27.260 | 1.00 | 0.00 | 0.127 | 0.00 | 0.00 |
| ATOM | 4534 | CH2 | TRP | 276 | 13.190 | 11.260 | 28.726 | 1.00 | 0.00 | −0.127 | 10.80 | 0.60 |
| ATOM | 4535 | HH2 | TRP | 276 | 12.413 | 11.386 | 27.971 | 1.00 | 0.00 | 0.127 | 0.00 | 0.00 |
| ATOM | 4536 | H | ALA | 277 | 17.683 | 12.507 | 29.867 | 1.00 | 0.00 | −0.650 | 9.00 | −17.40 |
| ATOM | 4537 | HN | ALA | 277 | 17.645 | 11.529 | 29.543 | 1.00 | 0.00 | 0.440 | 0.00 | 0.00 |
| ATOM | 4538 | CA | ALA | 277 | 17.336 | 13.583 | 28.999 | 1.00 | 0.00 | 0.158 | 9.40 | 4.00 |
| ATOM | 4539 | HA | ALA | 277 | 18.060 | 14.381 | 29.158 | 1.00 | 0.00 | 0.053 | 0.00 | 0.00 |
| ATOM | 4540 | C | ALA | 277 | 15.956 | 14.024 | 29.357 | 1.00 | 0.00 | 0.396 | 9.82 | 4.00 |
| ATOM | 4541 | O | ALA | 277 | 15.004 | 13.787 | 28.616 | 1.00 | 0.00 | −0.396 | 8.17 | −17.40 |
| ATOM | 4542 | CB | ALA | 277 | 17.336 | 13.187 | 27.514 | 1.00 | 0.00 | −0.159 | 16.15 | 4.00 |
| ATOM | 4543 | HB1 | ALA | 277 | 17.063 | 14.050 | 26.907 | 1.00 | 0.00 | 0.053 | 0.00 | 0.00 |
| ATOM | 4544 | HB2 | ALA | 277 | 18.330 | 12.842 | 27.229 | 1.00 | 0.00 | 0.053 | 0.00 | 0.00 |
| ATOM | 4545 | HB3 | ALA | 277 | 16.614 | 12.386 | 27.350 | 1.00 | 0.00 | 0.053 | 0.00 | 0.00 |
| ATOM | 4546 | N | THR | 278 | 15.817 | 14.676 | 30.525 | 1.00 | 0.00 | −0.650 | 9.00 | −17.40 |
| ATOM | 4547 | HN | THR | 278 | 16.637 | 14.815 | 31.132 | 1.00 | 0.00 | 0.440 | 0.00 | 0.00 |
| ATOM | 4548 | CA | THR | 278 | 14.543 | 15.177 | 30.931 | 1.00 | 0.00 | 0.158 | 9.40 | 4.00 |
| ATOM | 4549 | HA | THR | 278 | 13.818 | 14.415 | 30.640 | 1.00 | 0.00 | 0.053 | 0.00 | 0.00 |
| ATOM | 4550 | C | THR | 278 | 14.360 | 16.460 | 30.198 | 1.00 | 0.00 | 0.396 | 9.82 | 4.00 |
| ATOM | 4551 | O | THR | 278 | 15.333 | 17.139 | 29.873 | 1.00 | 0.00 | −0.396 | 8.17 | −17.40 |
| ATOM | 4552 | CB | THR | 278 | 14.477 | 15.524 | 32.382 | 1.00 | 0.00 | 0.060 | 9.40 | 4.00 |
| ATOM | 4553 | HB | THR | 278 | 14.851 | 14.663 | 32.936 | 1.00 | 0.00 | 0.053 | 0.00 | 0.00 |
| ATOM | 4554 | OG1 | THR | 278 | 13.140 | 15.805 | 32.767 | 1.00 | 0.00 | −0.537 | 11.04 | −17.40 |
| ATOM | 4555 | HG1 | THR | 278 | 13.008 | 16.824 | 32.833 | 1.00 | 0.00 | 0.424 | 0.00 | 0.00 |
| ATOM | 4556 | CG2 | THR | 278 | 15.362 | 16.764 | 32.594 | 1.00 | 0.00 | −0.159 | 16.15 | 4.00 |
| ATOM | 4557 | HG2 | THR | 278 | 15.340 | 17.050 | 33.645 | 1.00 | 0.00 | 0.053 | 0.00 | 0.00 |
| ATOM | 4558 | HG2 | THR | 278 | 16.386 | 16.534 | 32.301 | 1.00 | 0.00 | 0.053 | 0.00 | 0.00 |
| ATOM | 4559 | HG2 | THR | 278 | 14.986 | 17.587 | 31.985 | 1.00 | 0.00 | 0.053 | 0.00 | 0.00 |
| ATOM | 4560 | N | ILE | 279 | 13.100 | 16.825 | 29.908 | 1.00 | 0.00 | −0.650 | 9.00 | −17.40 |
| ATOM | 4561 | HN | ILE | 279 | 12.304 | 16.224 | 30.167 | 1.00 | 0.00 | 0.440 | 0.00 | 0.00 |
| ATOM | 4562 | CA | ILE | 279 | 12.888 | 18.067 | 29.230 | 1.00 | 0.00 | 0.158 | 9.40 | 4.00 |
| ATOM | 4563 | HA | ILE | 279 | 13.609 | 18.106 | 28.413 | 1.00 | 0.00 | 0.053 | 0.00 | 0.00 |
| ATOM | 4564 | C | ILE | 279 | 13.117 | 19.149 | 30.233 | 1.00 | 0.00 | 0.396 | 9.82 | 4.00 |
| ATOM | 4565 | O | ILE | 279 | 12.620 | 19.087 | 31.356 | 1.00 | 0.00 | −0.396 | 8.17 | −17.40 |
| ATOM | 4566 | CB | ILE | 279 | 11.500 | 18.224 | 28.673 | 1.00 | 0.00 | −0.053 | 9.40 | 4.00 |
| ATOM | 4567 | HB | ILE | 279 | 11.299 | 17.389 | 28.001 | 1.00 | 0.00 | 0.053 | 0.00 | 0.00 |
| ATOM | 4568 | CG1 | ILE | 279 | 11.423 | 19.471 | 27.776 | 1.00 | 0.00 | −0.106 | 12.77 | 4.00 |
| ATOM | 4569 | HG1 | ILE | 279 | 12.238 | 19.539 | 27.055 | 1.00 | 0.00 | 0.053 | 0.00 | 0.00 |
| ATOM | 4570 | HG1 | ILE | 279 | 11.453 | 20.408 | 28.331 | 1.00 | 0.00 | 0.053 | 0.00 | 0.00 |
| ATOM | 4571 | CG2 | ILE | 279 | 10.502 | 18.222 | 29.841 | 1.00 | 0.00 | −0.159 | 16.15 | 4.00 |
| ATOM | 4572 | HG2 | ILE | 279 | 9.489 | 18.335 | 29.454 | 1.00 | 0.00 | 0.053 | 0.00 | 0.00 |
| ATOM | 4573 | HG2 | ILE | 279 | 10.579 | 17.280 | 30.384 | 1.00 | 0.00 | 0.053 | 0.00 | 0.00 |
| ATOM | 4574 | HG2 | ILE | 279 | 10.727 | 19.049 | 30.514 | 1.00 | 0.00 | 0.053 | 0.00 | 0.00 |
| ATOM | 4575 | CD1 | ILE | 279 | 10.145 | 19.543 | 26.941 | 1.00 | 0.00 | −0.159 | 16.15 | 4.00 |
| ATOM | 4576 | HD1 | ILE | 279 | 10.158 | 20.447 | 26.332 | 1.00 | 0.00 | 0.053 | 0.00 | 0.00 |
| ATOM | 4577 | HD1 | ILE | 279 | 10.085 | 18.669 | 26.291 | 1.00 | 0.00 | 0.053 | 0.00 | 0.00 |
| ATOM | 4578 | HD1 | ILE | 279 | 9.278 | 19.563 | 27.602 | 1.00 | 0.00 | 0.053 | 0.00 | 0.00 |
| ATOM | 4579 | N | GLN | 280 | 13.927 | 20.153 | 29.853 | 1.00 | 0.00 | −0.650 | 9.00 | −17.40 |
| ATOM | 4580 | HN | GLN | 280 | 14.336 | 20.128 | 28.908 | 1.00 | 0.00 | 0.440 | 0.00 | 0.00 |
| ATOM | 4581 | CA | GLN | 280 | 14.242 | 21.257 | 30.713 | 1.00 | 0.00 | 0.158 | 9.40 | 4.00 |
| ATOM | 4582 | HA | GLN | 280 | 14.537 | 20.880 | 31.692 | 1.00 | 0.00 | 0.053 | 0.00 | 0.00 |
| ATOM | 4583 | C | GLN | 280 | 13.038 | 22.135 | 30.857 | 1.00 | 0.00 | 0.396 | 9.82 | 4.00 |
| ATOM | 4584 | O | GLN | 280 | 12.790 | 22.681 | 31.930 | 1.00 | 0.00 | −0.396 | 8.17 | −17.40 |
| ATOM | 4585 | CB | GLN | 280 | 15.383 | 22.129 | 30.165 | 1.00 | 0.00 | −0.106 | 12.77 | 4.00 |
| ATOM | 4586 | HB1 | GLN | 280 | 16.299 | 21.572 | 29.971 | 1.00 | 0.00 | 0.053 | 0.00 | 0.00 |
| ATOM | 4587 | HB2 | GLN | 280 | 15.674 | 22.934 | 30.838 | 1.00 | 0.00 | 0.053 | 0.00 | 0.00 |
| ATOM | 4588 | CG | GLN | 280 | 15.042 | 22.816 | 28.840 | 1.00 | 0.00 | −0.106 | 12.77 | 4.00 |
| ATOM | 4589 | HG1 | GLN | 280 | 14.172 | 23.452 | 29.004 | 1.00 | 0.00 | 0.053 | 0.00 | 0.00 |
| ATOM | 4590 | HG2 | GLN | 280 | 14.823 | 22.040 | 28.105 | 1.00 | 0.00 | 0.053 | 0.00 | 0.00 |
| ATOM | 4591 | CD | GLN | 280 | 16.248 | 23.643 | 28.415 | 1.00 | 0.00 | 0.396 | 9.82 | 4.00 |
| ATOM | 4592 | OE1 | GLN | 280 | 17.275 | 23.659 | 29.091 | 1.00 | 0.00 | −0.396 | 8.17 | −17.40 |
| ATOM | 4593 | NE2 | GLN | 280 | 16.122 | 24.350 | 27.260 | 1.00 | 0.00 | −0.879 | 13.25 | −17.40 |
| ATOM | 4594 | HE2 | GLN | 280 | 15.243 | 24.308 | 26.724 | 1.00 | 0.00 | 0.440 | 0.00 | 0.00 |
| ATOM | 4595 | HE2 | GLN | 280 | 16.905 | 24.927 | 26.921 | 1.00 | 0.00 | 0.440 | 0.00 | 0.00 |
| ATOM | 4596 | N | ARG | 281G | 12.261 | 22.282 | 29.765 | 1.00 | 0.00 | −0.650 | 9.00 | −17.40 |
| ATOM | 4597 | HN | ARG | 281G | 12.497 | 21.739 | 28.921 | 1.00 | 0.00 | 0.440 | 0.00 | 0.00 |
| ATOM | 4598 | CA | ARG | 281G | 11.120 | 23.156 | 29.716 | 1.00 | 0.00 | 0.158 | 9.40 | 4.00 |
| ATOM | 4599 | HA | ARG | 281G | 11.429 | 24.145 | 30.053 | 1.00 | 0.00 | 0.053 | 0.00 | 0.00 |
| ATOM | 4600 | C | ARG | 281G | 10.061 | 22.601 | 30.617 | 1.00 | 0.00 | 0.396 | 9.82 | 4.00 |
| ATOM | 4601 | O | ARG | 281G | 10.387 | 21.939 | 31.602 | 1.00 | 0.00 | −0.396 | 8.17 | −17.40 |
| ATOM | 4602 | CB | ARG | 281G | 10.537 | 23.296 | 28.296 | 1.00 | 0.00 | −0.106 | 12.77 | 4.00 |
| ATOM | 4603 | HB1 | ARG | 281G | 9.988 | 22.383 | 28.061 | 1.00 | 0.00 | 0.053 | 0.00 | 0.00 |
| ATOM | 4604 | HB2 | ARG | 281G | 11.364 | 23.437 | 27.600 | 1.00 | 0.00 | 0.053 | 0.00 | 0.00 |
| ATOM | 4605 | CG | ARG | 281G | 9.574 | 24.474 | 28.115 | 1.00 | 0.00 | −0.106 | 12.77 | 4.00 |
| ATOM | 4606 | HG1 | ARG | 281G | 10.040 | 25.445 | 28.279 | 1.00 | 0.00 | 0.053 | 0.00 | 0.00 |
| ATOM | 4607 | HG2 | ARG | 281G | 8.722 | 24.447 | 28.794 | 1.00 | 0.00 | 0.053 | 0.00 | 0.00 |
| ATOM | 4608 | CD | ARG | 281G | 8.966 | 24.554 | 26.714 | 1.00 | 0.00 | 0.374 | 12.77 | 4.00 |
| ATOM | 4609 | HD1 | ARG | 281G | 9.787 | 24.494 | 26.000 | 1.00 | 0.00 | 0.053 | 0.00 | 0.00 |
| ATOM | 4610 | HD2 | ARG | 281G | 8.441 | 25.506 | 26.642 | 1.00 | 0.00 | 0.053 | 0.00 | 0.00 |
| ATOM | 4611 | NE | ARG | 281G | 8.031 | 23.402 | 26.570 | 1.00 | 0.00 | −0.819 | 9.00 | −24.67 |
| ATOM | 4612 | HE | ARG | 281G | 7.923 | 22.727 | 27.341 | 1.00 | 0.00 | 0.407 | 0.00 | 0.00 |
| ATOM | 4613 | CZ | ARG | 281G | 7.318 | 23.240 | 25.418 | 1.00 | 0.00 | 0.796 | 6.95 | 4.00 |
| ATOM | 4614 | NH1 | ARG | 281G | 7.463 | 24.131 | 24.395 | 1.00 | 0.00 | −0.746 | 9.00 | −24.67 |
| ATOM | 4615 | HH1 | ARG | 281G | 6.924 | 24.008 | 23.525 | 1.00 | 0.00 | 0.407 | 0.00 | 0.00 |
| ATOM | 4616 | HH1 | ARG | 281G | 8.110 | 24.926 | 24.492 | 1.00 | 0.00 | 0.407 | 0.00 | 0.00 |
| ATOM | 4617 | NH2 | ARG | 281G | 6.460 | 22.185 | 25.289 | 1.00 | 0.00 | −0.746 | 9.00 | −24.67 |
| ATOM | 4618 | HH2 | ARG | 281G | 5.921 | 22.062 | 24.419 | 1.00 | 0.00 | 0.407 | 0.00 | 0.00 |
| ATOM | 4619 | HH2 | ARG | 281G | 6.351 | 21.512 | 26.061 | 1.00 | 0.00 | 0.407 | 0.00 | 0.00 |
| ATOM | 4620 | N | ILE | 282 | 8.772 | 22.910 | 30.319 | 1.00 | 0.00 | −0.650 | 9.00 | −17.40 |
| ATOM | 4621 | HN | ILE | 282 | 8.585 | 23.515 | 29.507 | 1.00 | 0.00 | 0.440 | 0.00 | 0.00 |
| ATOM | 4622 | CA | ILE | 282 | 7.656 | 22.428 | 31.088 | 1.00 | 0.00 | 0.158 | 9.40 | 4.00 |
| ATOM | 4623 | HA | ILE | 282 | 7.703 | 22.963 | 32.036 | 1.00 | 0.00 | 0.053 | 0.00 | 0.00 |
| ATOM | 4624 | C | ILE | 282 | 7.876 | 20.966 | 31.231 | 1.00 | 0.00 | 0.396 | 9.82 | 4.00 |
| ATOM | 4625 | O | ILE | 282 | 8.213 | 20.272 | 30.274 | 1.00 | 0.00 | −0.396 | 8.17 | −17.40 |
| ATOM | 4626 | CB | ILE | 282 | 6.322 | 22.676 | 30.432 | 1.00 | 0.00 | −0.053 | 9.40 | 4.00 |
| ATOM | 4627 | HB | ILE | 282 | 6.251 | 23.736 | 30.192 | 1.00 | 0.00 | 0.053 | 0.00 | 0.00 |
| ATOM | 4628 | CG1 | ILE | 282 | 5.157 | 22.445 | 31.414 | 1.00 | 0.00 | −0.106 | 12.77 | 4.00 |
| ATOM | 4629 | HG1 | ILE | 282 | 4.228 | 22.714 | 30.910 | 1.00 | 0.00 | 0.053 | 0.00 | 0.00 |
| ATOM | 4630 | HG1 | ILE | 282 | 5.318 | 23.075 | 32.288 | 1.00 | 0.00 | 0.053 | 0.00 | 0.00 |
| ATOM | 4631 | CG2 | ILE | 282 | 6.248 | 21.825 | 29.154 | 1.00 | 0.00 | −0.159 | 16.15 | 4.00 |
| ATOM | 4632 | HG2 | ILE | 282 | 5.289 | 21.989 | 28.662 | 1.00 | 0.00 | 0.053 | 0.00 | 0.00 |
| ATOM | 4633 | HG2 | ILE | 282 | 7.055 | 22.110 | 28.479 | 1.00 | 0.00 | 0.053 | 0.00 | 0.00 |
| ATOM | 4634 | HG2 | ILE | 282 | 6.347 | 20.770 | 29.412 | 1.00 | 0.00 | 0.053 | 0.00 | 0.00 |
| ATOM | 4635 | CD1 | ILE | 282 | 5.011 | 21.005 | 31.907 | 1.00 | 0.00 | −0.159 | 16.15 | 4.00 |
| ATOM | 4636 | HD1 | ILE | 282 | 4.166 | 20.939 | 32.592 | 1.00 | 0.00 | 0.053 | 0.00 | 0.00 |
| ATOM | 4637 | HD1 | ILE | 282 | 4.840 | 20.344 | 31.056 | 1.00 | 0.00 | 0.053 | 0.00 | 0.00 |
| ATOM | 4638 | HD1 | ILE | 282 | 5.922 | 20.703 | 32.423 | 1.00 | 0.00 | 0.053 | 0.00 | 0.00 |
| ATOM | 4639 | N | PRO | 283 | 7.727 | 20.490 | 32.427 | 1.00 | 0.00 | −0.422 | 9.00 | −17.40 |
| ATOM | 4640 | CA | PRO | 283 | 8.054 | 19.120 | 32.640 | 1.00 | 0.00 | 0.158 | 9.40 | 4.00 |
| ATOM | 4641 | HA | PRO | 283 | 9.063 | 18.880 | 32.305 | 1.00 | 0.00 | 0.053 | 0.00 | 0.00 |
| ATOM | 4642 | CD | PRO | 283 | 7.989 | 21.301 | 33.605 | 1.00 | 0.00 | 0.105 | 12.77 | 4.00 |
| ATOM | 4643 | HD1 | PRO | 283 | 7.026 | 21.734 | 33.878 | 1.00 | 0.00 | 0.053 | 0.00 | 0.00 |
| ATOM | 4644 | HD2 | PRO | 283 | 8.720 | 22.044 | 33.288 | 1.00 | 0.00 | 0.053 | 0.00 | 0.00 |
| ATOM | 4645 | C | PRO | 283 | 7.139 | 18.206 | 31.914 | 1.00 | 0.00 | 0.396 | 9.82 | 4.00 |
| ATOM | 4646 | O | PRO | 283 | 5.925 | 18.392 | 31.973 | 1.00 | 0.00 | −0.396 | 8.17 | −17.40 |
| ATOM | 4647 | CB | PRO | 283 | 8.110 | 18.932 | 34.155 | 1.00 | 0.00 | −0.106 | 12.77 | 4.00 |
| ATOM | 4648 | HB1 | PRO | 283 | 8.830 | 18.161 | 34.428 | 1.00 | 0.00 | 0.053 | 0.00 | 0.00 |
| ATOM | 4649 | HB2 | PRO | 283 | 7.138 | 18.634 | 34.550 | 1.00 | 0.00 | 0.053 | 0.00 | 0.00 |
| ATOM | 4650 | CG | PRO | 283 | 8.536 | 20.324 | 34.661 | 1.00 | 0.00 | −0.106 | 12.77 | 4.00 |
| ATOM | 4651 | HG1 | PRO | 283 | 9.624 | 20.278 | 34.700 | 1.00 | 0.00 | 0.053 | 0.00 | 0.00 |
| ATOM | 4652 | HG2 | PRO | 283 | 8.060 | 20.415 | 35.637 | 1.00 | 0.00 | 0.053 | 0.00 | 0.00 |
| ATOM | 4653 | N | GLU | 284 | 7.717 | 17.220 | 31.209 | 1.00 | 0.00 | −0.650 | 9.00 | −17.40 |
| ATOM | 4654 | HN | GLU | 284 | 8.743 | 17.168 | 31.142 | 1.00 | 0.00 | 0.440 | 0.00 | 0.00 |
| ATOM | 4655 | CA | GLU | 284 | 6.911 | 16.246 | 30.553 | 1.00 | 0.00 | 0.158 | 9.40 | 4.00 |
| ATOM | 4656 | HA | GLU | 284 | 6.049 | 16.709 | 30.071 | 1.00 | 0.00 | 0.053 | 0.00 | 0.00 |
| ATOM | 4657 | C | GLU | 284 | 6.507 | 15.341 | 31.663 | 1.00 | 0.00 | 0.396 | 9.82 | 4.00 |
| ATOM | 4658 | O | GLU | 284 | 7.258 | 15.212 | 32.627 | 1.00 | 0.00 | −0.396 | 8.17 | −17.40 |
| ATOM | 4659 | CB | GLU | 284 | 7.693 | 15.389 | 29.543 | 1.00 | 0.00 | −0.106 | 12.77 | 4.00 |
| ATOM | 4660 | HB1 | GLU | 284 | 7.097 | 14.603 | 29.078 | 1.00 | 0.00 | 0.053 | 0.00 | 0.00 |
| ATOM | 4661 | HB2 | GLU | 284 | 8.549 | 14.871 | 29.975 | 1.00 | 0.00 | 0.053 | 0.00 | 0.00 |
| ATOM | 4662 | CG | GLU | 284 | 8.270 | 16.188 | 28.370 | 1.00 | 0.00 | −0.106 | 12.77 | 4.00 |
| ATOM | 4663 | HG1 | GLU | 284 | 8.929 | 16.956 | 28.774 | 1.00 | 0.00 | 0.053 | 0.00 | 0.00 |
| ATOM | 4664 | HG2 | GLU | 284 | 7.439 | 16.637 | 27.825 | 1.00 | 0.00 | 0.053 | 0.00 | 0.00 |
| ATOM | 4665 | CD | GLU | 284 | 9.043 | 15.228 | 27.479 | 1.00 | 0.00 | 0.399 | 9.82 | 4.00 |
| ATOM | 4666 | OE1 | GLU | 284 | 9.813 | 14.397 | 28.031 | 1.00 | 0.00 | −0.396 | 8.17 | −18.95 |
| ATOM | 4667 | OE2 | GLU | 284 | 8.873 | 15.311 | 26.233 | 1.00 | 0.00 | −0.427 | 8.17 | −18.95 |
| ATOM | 4669 | N | VAL | 285 | 5.298 | 14.742 | 31.580 | 1.00 | 0.00 | −0.650 | 9.00 | −17.40 |
| ATOM | 4670 | HN | VAL | 285 | 4.682 | 14.946 | 30.780 | 1.00 | 0.00 | 0.440 | 0.00 | 0.00 |
| ATOM | 4671 | CA | VAL | 285 | 4.862 | 13.823 | 32.595 | 1.00 | 0.00 | 0.158 | 9.40 | 4.00 |
| ATOM | 4672 | HA | VAL | 285 | 4.025 | 13.283 | 32.151 | 1.00 | 0.00 | 0.053 | 0.00 | 0.00 |
| ATOM | 4673 | C | VAL | 285 | 4.465 | 14.684 | 33.762 | 1.00 | 0.00 | 0.396 | 9.82 | 4.00 |
| ATOM | 4674 | O | VAL | 285 | 4.484 | 15.910 | 33.631 | 1.00 | 0.00 | −0.396 | 8.17 | −17.40 |
| ATOM | 4675 | CB | VAL | 285 | 5.970 | 12.831 | 32.921 | 1.00 | 0.00 | −0.053 | 9.40 | 4.00 |
| ATOM | 4676 | HB | VAL | 285 | 6.838 | 13.390 | 33.268 | 1.00 | 0.00 | 0.053 | 0.00 | 0.00 |
| ATOM | 4677 | CG1 | VAL | 285 | 5.605 | 11.815 | 34.008 | 1.00 | 0.00 | −0.159 | 16.15 | 4.00 |
| ATOM | 4678 | HG1 | VAL | 285 | 6.448 | 11.146 | 34.179 | 1.00 | 0.00 | 0.053 | 0.00 | 0.00 |
| ATOM | 4679 | HG1 | VAL | 285 | 5.365 | 12.341 | 34.932 | 1.00 | 0.00 | 0.053 | 0.00 | 0.00 |
| ATOM | 4680 | HG1 | VAL | 285 | 4.740 | 11.233 | 33.687 | 1.00 | 0.00 | 0.053 | 0.00 | 0.00 |
| ATOM | 4681 | CG2 | VAL | 285 | 6.353 | 12.166 | 31.595 | 1.00 | 0.00 | −0.159 | 16.15 | 4.00 |
| ATOM | 4682 | HG2 | VAL | 285 | 7.148 | 11.440 | 31.768 | 1.00 | 0.00 | 0.053 | 0.00 | 0.00 |
| ATOM | 4683 | HG2 | VAL | 285 | 5.483 | 11.658 | 31.177 | 1.00 | 0.00 | 0.053 | 0.00 | 0.00 |
| ATOM | 4684 | HG2 | VAL | 285 | 6.700 | 12.925 | 30.894 | 1.00 | 0.00 | 0.053 | 0.00 | 0.00 |
| ATOM | 4685 | N | PRO | 286 | 4.084 | 14.157 | 34.899 | 1.00 | 0.00 | −0.422 | 9.00 | −17.40 |
| ATOM | 4686 | CA | PRO | 286 | 3.836 | 15.053 | 35.996 | 1.00 | 0.00 | 0.158 | 9.40 | 4.00 |
| ATOM | 4687 | HA | PRO | 286 | 3.231 | 15.901 | 35.675 | 1.00 | 0.00 | 0.053 | 0.00 | 0.00 |
| ATOM | 4688 | CD | PRO | 286 | 3.059 | 13.120 | 34.898 | 1.00 | 0.00 | 0.105 | 12.77 | 4.00 |
| ATOM | 4689 | HD1 | PRO | 286 | 3.415 | 12.216 | 34.403 | 1.00 | 0.00 | 0.053 | 0.00 | 0.00 |
| ATOM | 4690 | HD2 | PRO | 286 | 2.162 | 13.454 | 34.375 | 1.00 | 0.00 | 0.053 | 0.00 | 0.00 |
| ATOM | 4691 | C | PRO | 286 | 5.117 | 15.581 | 36.551 | 1.00 | 0.00 | 0.396 | 9.82 | 4.00 |
| ATOM | 4692 | O | PRO | 286 | 5.099 | 16.144 | 37.644 | 1.00 | 0.00 | −0.396 | 8.17 | −17.40 |
| ATOM | 4693 | CB | PRO | 286 | 2.986 | 14.283 | 37.010 | 1.00 | 0.00 | −0.106 | 12.77 | 4.00 |
| ATOM | 4694 | HB1 | PRO | 286 | 2.060 | 14.853 | 37.091 | 1.00 | 0.00 | 0.053 | 0.00 | 0.00 |
| ATOM | 4695 | HB2 | PRO | 286 | 3.577 | 14.277 | 37.925 | 1.00 | 0.00 | 0.053 | 0.00 | 0.00 |
| ATOM | 4696 | CG | PRO | 286 | 2.797 | 12.888 | 36.390 | 1.00 | 0.00 | −0.106 | 12.77 | 4.00 |
| ATOM | 4697 | HG1 | PRO | 286 | 1.788 | 12.514 | 36.569 | 1.00 | 0.00 | 0.053 | 0.00 | 0.00 |
| ATOM | 4698 | HG2 | PRO | 286 | 3.500 | 12.172 | 36.816 | 1.00 | 0.00 | 0.053 | 0.00 | 0.00 |
| ATOM | 4699 | N | GLY | 287 | 6.233 | 15.438 | 35.808 | 1.00 | 0.00 | −0.650 | 9.00 | −17.40 |
| ATOM | 4700 | HN | GLY | 287 | 6.183 | 14.942 | 34.906 | 1.00 | 0.00 | 0.440 | 0.00 | 0.00 |
| ATOM | 4701 | CA | GLY | 287 | 7.483 | 15.965 | 36.254 | 1.00 | 0.00 | 0.105 | 9.40 | 4.00 |
| ATOM | 4702 | HA1 | GLY | 287 | 7.294 | 16.686 | 37.049 | 1.00 | 0.00 | 0.053 | 0.00 | 0.00 |
| ATOM | 4703 | HA2 | GLY | 287 | 7.977 | 16.451 | 35.413 | 1.00 | 0.00 | 0.053 | 0.00 | 0.00 |
| ATOM | 4704 | C | GLY | 287 | 8.319 | 14.844 | 36.764 | 1.00 | 0.00 | 0.396 | 9.82 | 4.00 |
| ATOM | 4705 | O | GLY | 287 | 9.389 | 15.088 | 37.317 | 1.00 | 0.00 | −0.396 | 8.17 | −17.40 |
| ATOM | 4706 | N | SER | 288 | 7.864 | 13.584 | 36.607 | 1.00 | 0.00 | −0.650 | 9.00 | −17.40 |
| ATOM | 4707 | HN | SER | 288 | 6.974 | 13.384 | 36.127 | 1.00 | 0.00 | 0.440 | 0.00 | 0.00 |
| ATOM | 4708 | CA | SER | 288 | 8.685 | 12.537 | 37.143 | 1.00 | 0.00 | 0.158 | 9.40 | 4.00 |
| ATOM | 4709 | HA | SER | 288 | 9.721 | 12.859 | 37.239 | 1.00 | 0.00 | 0.053 | 0.00 | 0.00 |
| ATOM | 4710 | C | SER | 288 | 8.665 | 11.321 | 36.255 | 1.00 | 0.00 | 0.396 | 9.82 | 4.00 |
| ATOM | 4711 | O | SER | 288 | 7.597 | 10.839 | 35.887 | 1.00 | 0.00 | −0.396 | 8.17 | −17.40 |
| ATOM | 4712 | CB | SER | 288 | 8.191 | 12.085 | 38.527 | 1.00 | 0.00 | 0.007 | 12.77 | 4.00 |
| ATOM | 4713 | HB1 | SER | 288 | 8.830 | 11.291 | 38.914 | 1.00 | 0.00 | 0.053 | 0.00 | 0.00 |
| ATOM | 4714 | HB2 | SER | 288 | 7.170 | 11.709 | 38.458 | 1.00 | 0.00 | 0.053 | 0.00 | 0.00 |
| ATOM | 4715 | OG | SER | 288 | 8.215 | 13.175 | 39.437 | 1.00 | 0.00 | −0.537 | 11.04 | −17.40 |
| ATOM | 4716 | HG | SER | 288 | 8.966 | 13.828 | 39.171 | 1.00 | 0.00 | 0.424 | 0.00 | 0.00 |
| ATOM | 4717 | N | LEU | 289 | 9.860 | 10.817 | 35.854 | 1.00 | 0.00 | −0.650 | 9.00 | −17.40 |
| ATOM | 4718 | HN | LEU | 289 | 10.712 | 11.361 | 36.048 | 1.00 | 0.00 | 0.440 | 0.00 | 0.00 |
| ATOM | 4719 | CA | LEU | 289 | 9.991 | 9.554 | 35.168 | 1.00 | 0.00 | 0.158 | 9.40 | 4.00 |
| ATOM | 4720 | HA | LEU | 289 | 9.659 | 8.740 | 35.813 | 1.00 | 0.00 | 0.053 | 0.00 | 0.00 |
| ATOM | 4721 | C | LEU | 289 | 11.425 | 9.319 | 34.789 | 1.00 | 0.00 | 0.396 | 9.82 | 4.00 |
| ATOM | 4722 | O | LEU | 289 | 11.947 | 10.001 | 33.911 | 1.00 | 0.00 | −0.396 | 8.17 | −17.40 |
| ATOM | 4723 | CB | LEU | 289 | 9.218 | 9.423 | 33.853 | 1.00 | 0.00 | −0.106 | 12.77 | 4.00 |
| ATOM | 4724 | HB1 | LEU | 289 | 9.542 | 10.174 | 33.133 | 1.00 | 0.00 | 0.053 | 0.00 | 0.00 |
| ATOM | 4725 | HB2 | LEU | 289 | 8.148 | 9.553 | 34.015 | 1.00 | 0.00 | 0.053 | 0.00 | 0.00 |
| ATOM | 4726 | CG | LEU | 289 | 9.437 | 8.032 | 33.220 | 1.00 | 0.00 | −0.053 | 9.40 | 4.00 |
| ATOM | 4727 | HG | LEU | 289 | 10.495 | 7.772 | 33.186 | 1.00 | 0.00 | 0.053 | 0.00 | 0.00 |
| ATOM | 4728 | CD1 | LEU | 289 | 8.721 | 6.938 | 34.014 | 1.00 | 0.00 | −0.159 | 16.15 | 4.00 |
| ATOM | 4729 | HD1 | LEU | 289 | 8.895 | 5.971 | 33.541 | 1.00 | 0.00 | 0.053 | 0.00 | 0.00 |
| ATOM | 4730 | HD1 | LEU | 289 | 9.105 | 6.917 | 35.033 | 1.00 | 0.00 | 0.053 | 0.00 | 0.00 |
| ATOM | 4731 | HD1 | LEU | 289 | 7.650 | 7.143 | 34.033 | 1.00 | 0.00 | 0.053 | 0.00 | 0.00 |
| ATOM | 4732 | CD2 | LEU | 289 | 9.130 | 8.017 | 31.712 | 1.00 | 0.00 | −0.159 | 16.15 | 4.00 |
| ATOM | 4733 | HD2 | LEU | 289 | 9.299 | 7.015 | 31.316 | 1.00 | 0.00 | 0.053 | 0.00 | 0.00 |
| ATOM | 4734 | HD2 | LEU | 289 | 8.089 | 8.299 | 31.549 | 1.00 | 0.00 | 0.053 | 0.00 | 0.00 |
| ATOM | 4735 | HD2 | LEU | 289 | 9.782 | 8.724 | 31.200 | 1.00 | 0.00 | 0.053 | 0.00 | 0.00 |
| ATOM | 4736 | N | PRO | 290 | 12.086 | 8.382 | 35.414 | 1.00 | 0.00 | −0.422 | 9.00 | −17.40 |
| ATOM | 4737 | CA | PRO | 290 | 13.410 | 8.034 | 34.962 | 1.00 | 0.00 | 0.158 | 9.40 | 4.00 |
| ATOM | 4738 | HA | PRO | 290 | 13.831 | 8.937 | 34.520 | 1.00 | 0.00 | 0.053 | 0.00 | 0.00 |
| ATOM | 4739 | CD | PRO | 290 | 11.994 | 8.312 | 36.861 | 1.00 | 0.00 | 0.105 | 12.77 | 4.00 |
| ATOM | 4740 | HD1 | PRO | 290 | 11.014 | 7.875 | 37.056 | 1.00 | 0.00 | 0.053 | 0.00 | 0.00 |
| ATOM | 4741 | HD2 | PRO | 290 | 12.088 | 9.345 | 37.195 | 1.00 | 0.00 | 0.053 | 0.00 | 0.00 |
| ATOM | 4742 | C | PRO | 290 | 13.239 | 6.940 | 33.963 | 1.00 | 0.00 | 0.396 | 9.82 | 4.00 |
| ATOM | 4743 | O | PRO | 290 | 12.116 | 6.461 | 33.823 | 1.00 | 0.00 | −0.396 | 8.17 | −17.40 |
| ATOM | 4744 | CB | PRO | 290 | 14.221 | 7.639 | 36.196 | 1.00 | 0.00 | −0.106 | 12.77 | 4.00 |
| ATOM | 4745 | HB1 | PRO | 290 | 14.871 | 8.494 | 36.375 | 1.00 | 0.00 | 0.053 | 0.00 | 0.00 |
| ATOM | 4746 | HB2 | PRO | 290 | 14.747 | 6.734 | 35.893 | 1.00 | 0.00 | 0.053 | 0.00 | 0.00 |
| ATOM | 4747 | CG | PRO | 290 | 13.167 | 7.415 | 37.285 | 1.00 | 0.00 | −0.106 | 12.77 | 4.00 |
| ATOM | 4748 | HG1 | PRO | 290 | 13.662 | 7.730 | 38.203 | 1.00 | 0.00 | 0.053 | 0.00 | 0.00 |
| ATOM | 4749 | HG2 | PRO | 290 | 12.955 | 6.347 | 37.234 | 1.00 | 0.00 | 0.053 | 0.00 | 0.00 |
| ATOM | 4750 | N | ALA | 291 | 14.317 | 6.521 | 33.268 | 1.00 | 0.00 | −0.650 | 9.00 | −17.40 |
| ATOM | 4751 | HN | ALA | 291 | 15.250 | 6.905 | 33.473 | 1.00 | 0.00 | 0.440 | 0.00 | 0.00 |
| ATOM | 4752 | CA | ALA | 291 | 14.151 | 5.535 | 32.240 | 1.00 | 0.00 | 0.158 | 9.40 | 4.00 |
| ATOM | 4753 | HA | ALA | 291 | 13.542 | 5.937 | 31.430 | 1.00 | 0.00 | 0.053 | 0.00 | 0.00 |
| ATOM | 4754 | C | ALA | 291 | 13.474 | 4.321 | 32.797 | 1.00 | 0.00 | 0.396 | 9.82 | 4.00 |
| ATOM | 4755 | O | ALA | 291 | 12.276 | 4.130 | 32.602 | 1.00 | 0.00 | −0.396 | 8.17 | −17.40 |
| ATOM | 4756 | CB | ALA | 291 | 15.488 | 5.081 | 31.630 | 1.00 | 0.00 | −0.159 | 16.15 | 4.00 |
| ATOM | 4757 | HB1 | ALA | 291 | 15.300 | 4.334 | 30.857 | 1.00 | 0.00 | 0.053 | 0.00 | 0.00 |
| ATOM | 4758 | HB2 | ALA | 291 | 15.997 | 5.938 | 31.190 | 1.00 | 0.00 | 0.053 | 0.00 | 0.00 |
| ATOM | 4759 | HB3 | ALA | 291 | 16.114 | 4.647 | 32.409 | 1.00 | 0.00 | 0.053 | 0.00 | 0.00 |
| ATOM | 4760 | N | SER | 292 | 14.216 | 3.468 | 33.524 | 1.00 | 0.00 | −0.650 | 9.00 | −17.40 |
| ATOM | 4761 | HN | SER | 292 | 15.213 | 3.662 | 33.690 | 1.00 | 0.00 | 0.440 | 0.00 | 0.00 |
| ATOM | 4762 | CA | SER | 292 | 13.611 | 2.285 | 34.068 | 1.00 | 0.00 | 0.158 | 9.40 | 4.00 |
| ATOM | 4763 | HA | SER | 292 | 12.863 | 1.912 | 33.367 | 1.00 | 0.00 | 0.053 | 0.00 | 0.00 |
| ATOM | 4764 | C | SER | 292 | 12.963 | 2.622 | 35.369 | 1.00 | 0.00 | 0.396 | 9.82 | 4.00 |
| ATOM | 4765 | O | SER | 292 | 11.870 | 2.156 | 35.688 | 1.00 | 0.00 | −0.396 | 8.17 | −17.40 |
| ATOM | 4766 | CB | SER | 292 | 14.633 | 1.170 | 34.344 | 1.00 | 0.00 | 0.007 | 12.77 | 4.00 |
| ATOM | 4767 | HB1 | SER | 292 | 15.193 | 0.943 | 33.436 | 1.00 | 0.00 | 0.053 | 0.00 | 0.00 |
| ATOM | 4768 | HB2 | SER | 292 | 14.120 | 0.267 | 34.676 | 1.00 | 0.00 | 0.053 | 0.00 | 0.00 |
| ATOM | 4769 | OG | SER | 292 | 15.542 | 1.582 | 35.355 | 1.00 | 0.00 | −0.537 | 11.04 | −17.40 |
| ATOM | 4770 | HG | SER | 292 | 16.043 | 2.425 | 35.043 | 1.00 | 0.00 | 0.424 | 0.00 | 0.00 |
| ATOM | 4771 | N | HIS | 293S | 13.652 | 3.476 | 36.140 | 1.00 | 0.00 | −0.650 | 9.00 | −17.40 |
| ATOM | 4772 | HN | HIS | 293S | 14.495 | 3.901 | 35.728 | 1.00 | 0.00 | 0.440 | 0.00 | 0.00 |
| ATOM | 4773 | CA | HIS | 293S | 13.324 | 3.852 | 37.484 | 1.00 | 0.00 | 0.158 | 9.40 | 4.00 |
| ATOM | 4774 | HA | HIS | 293S | 13.243 | 2.968 | 38.117 | 1.00 | 0.00 | 0.053 | 0.00 | 0.00 |
| ATOM | 4775 | C | HIS | 293S | 12.015 | 4.583 | 37.519 | 1.00 | 0.00 | 0.396 | 9.82 | 4.00 |
| ATOM | 4776 | O | HIS | 293S | 11.596 | 5.203 | 36.543 | 1.00 | 0.00 | −0.396 | 8.17 | −17.40 |
| ATOM | 4777 | CB | HIS | 293S | 14.434 | 4.766 | 38.038 | 1.00 | 0.00 | −0.106 | 12.77 | 4.00 |
| ATOM | 4778 | HB1 | HIS | 293S | 14.443 | 5.672 | 37.432 | 1.00 | 0.00 | 0.053 | 0.00 | 0.00 |
| ATOM | 4779 | HB2 | HIS | 293S | 15.375 | 4.222 | 37.956 | 1.00 | 0.00 | 0.053 | 0.00 | 0.00 |
| ATOM | 4780 | CG | HIS | 293S | 14.326 | 5.212 | 39.464 | 1.00 | 0.00 | −0.050 | 7.26 | 0.60 |
| ATOM | 4781 | ND1 | HIS | 293S | 15.190 | 4.835 | 40.468 | 1.00 | 0.00 | 0.207 | 9.25 | −17.40 |
| ATOM | 4782 | HD1 | HIS | 293S | 15.975 | 4.173 | 40.386 | 1.00 | 0.00 | 0.393 | 0.00 | 0.00 |
| ATOM | 4783 | CD2 | HIS | 293S | 13.458 | 6.084 | 40.036 | 1.00 | 0.00 | −0.177 | 10.80 | 0.60 |
| ATOM | 4784 | HD2 | HIS | 293S | 12.633 | 6.568 | 39.512 | 1.00 | 0.00 | 0.127 | 0.00 | 0.00 |
| ATOM | 4785 | CE1 | HIS | 293S | 14.806 | 5.504 | 41.586 | 1.00 | 0.00 | −0.227 | 10.80 | 0.60 |
| ATOM | 4786 | HE1 | HIS | 293S | 15.306 | 5.413 | 42.550 | 1.00 | 0.00 | 0.127 | 0.00 | 0.00 |
| ATOM | 4787 | NE2 | HIS | 293S | 13.759 | 6.275 | 41.371 | 1.00 | 0.00 | 0.207 | 9.25 | −17.40 |
| ATOM | 4788 | HE2 | HIS | 293S | 13.274 | 6.883 | 42.046 | 1.00 | 0.00 | 0.393 | 0.00 | 0.00 |
| ATOM | 4789 | N | LYS | 294S | 11.306 | 4.473 | 38.662 | 1.00 | 0.00 | −0.650 | 9.00 | −17.40 |
| ATOM | 4790 | HN | LYS | 294S | 11.627 | 3.831 | 39.400 | 1.00 | 0.00 | 0.440 | 0.00 | 0.00 |
| ATOM | 4791 | CA | LYS | 294S | 10.112 | 5.239 | 38.859 | 1.00 | 0.00 | 0.158 | 9.40 | 4.00 |
| ATOM | 4792 | HA | LYS | 294S | 9.881 | 5.648 | 37.875 | 1.00 | 0.00 | 0.053 | 0.00 | 0.00 |
| ATOM | 4793 | C | LYS | 294S | 10.486 | 6.267 | 39.864 | 1.00 | 0.00 | 0.396 | 9.82 | 4.00 |
| ATOM | 4794 | O | LYS | 294S | 10.512 | 6.005 | 41.063 | 1.00 | 0.00 | −0.396 | 8.17 | −17.40 |
| ATOM | 4795 | CB | LYS | 294S | 8.919 | 4.450 | 39.423 | 1.00 | 0.00 | −0.106 | 12.77 | 4.00 |
| ATOM | 4796 | HB1 | LYS | 294S | 9.140 | 4.008 | 40.394 | 1.00 | 0.00 | 0.053 | 0.00 | 0.00 |
| ATOM | 4797 | HB2 | LYS | 294S | 8.621 | 3.630 | 38.768 | 1.00 | 0.00 | 0.053 | 0.00 | 0.00 |
| ATOM | 4798 | CG | LYS | 294S | 7.682 | 5.331 | 39.613 | 1.00 | 0.00 | −0.106 | 12.77 | 4.00 |
| ATOM | 4799 | HG1 | LYS | 294S | 7.842 | 6.156 | 40.306 | 1.00 | 0.00 | 0.053 | 0.00 | 0.00 |
| ATOM | 4800 | HG2 | LYS | 294S | 6.821 | 4.788 | 40.004 | 1.00 | 0.00 | 0.053 | 0.00 | 0.00 |
| ATOM | 4801 | CD | LYS | 294S | 7.195 | 5.981 | 38.314 | 1.00 | 0.00 | −0.106 | 12.77 | 4.00 |
| ATOM | 4802 | HD1 | LYS | 294S | 7.963 | 6.673 | 37.968 | 1.00 | 0.00 | 0.053 | 0.00 | 0.00 |
| ATOM | 4803 | HD2 | LYS | 294S | 6.266 | 6.511 | 38.524 | 1.00 | 0.00 | 0.053 | 0.00 | 0.00 |
| ATOM | 4804 | CE | LYS | 294S | 6.918 | 4.991 | 37.179 | 1.00 | 0.00 | 0.099 | 12.77 | 4.00 |
| ATOM | 4805 | HE1 | LYS | 294S | 7.544 | 4.105 | 37.291 | 1.00 | 0.00 | 0.053 | 0.00 | 0.00 |
| ATOM | 4806 | HE2 | LYS | 294S | 7.134 | 5.453 | 36.216 | 1.00 | 0.00 | 0.053 | 0.00 | 0.00 |
| ATOM | 4807 | NZ | LYS | 294S | 5.498 | 4.576 | 37.200 | 1.00 | 0.00 | −0.045 | 13.25 | −39.20 |
| ATOM | 4808 | HZ1 | LYS | 294S | 5.322 | 3.910 | 36.433 | 1.00 | 0.00 | 0.280 | 0.00 | 0.00 |
| ATOM | 4809 | HZ2 | LYS | 294S | 5.285 | 4.124 | 38.100 | 1.00 | 0.00 | 0.280 | 0.00 | 0.00 |
| ATOM | 4810 | HZ3 | LYS | 294S | 4.896 | 5.403 | 37.080 | 1.00 | 0.00 | 0.280 | 0.00 | 0.00 |
| ATOM | 4811 | N | TYR | 295 | 10.808 | 7.477 | 39.381 | 1.00 | 0.00 | −0.650 | 9.00 | −17.40 |
| ATOM | 4812 | HN | TYR | 295 | 10.700 | 7.666 | 38.374 | 1.00 | 0.00 | 0.440 | 0.00 | 0.00 |
| ATOM | 4813 | CA | TYR | 295 | 11.298 | 8.507 | 40.243 | 1.00 | 0.00 | 0.158 | 9.40 | 4.00 |
| ATOM | 4814 | HA | TYR | 295 | 11.338 | 8.135 | 41.267 | 1.00 | 0.00 | 0.053 | 0.00 | 0.00 |
| ATOM | 4815 | C | TYR | 295 | 10.393 | 9.678 | 40.178 | 1.00 | 0.00 | 0.396 | 9.82 | 4.00 |
| ATOM | 4816 | O | TYR | 295 | 9.763 | 9.936 | 39.158 | 1.00 | 0.00 | −0.396 | 8.17 | −17.40 |
| ATOM | 4817 | CB | TYR | 295 | 12.711 | 8.961 | 39.843 | 1.00 | 0.00 | −0.106 | 12.77 | 4.00 |
| ATOM | 4818 | HB1 | TYR | 295 | 12.818 | 8.980 | 38.758 | 1.00 | 0.00 | 0.053 | 0.00 | 0.00 |
| ATOM | 4819 | HB2 | TYR | 295 | 13.463 | 8.283 | 40.246 | 1.00 | 0.00 | 0.053 | 0.00 | 0.00 |
| ATOM | 4820 | CG | TYR | 295 | 12.992 | 10.322 | 40.358 | 1.00 | 0.00 | 0.000 | 7.26 | 0.60 |
| ATOM | 4821 | CD1 | TYR | 295 | 13.074 | 10.580 | 41.701 | 1.00 | 0.00 | −0.127 | 10.80 | 0.60 |
| ATOM | 4822 | HD1 | TYR | 295 | 12.922 | 9.779 | 42.425 | 1.00 | 0.00 | 0.127 | 0.00 | 0.00 |
| ATOM | 4823 | CD2 | TYR | 295 | 13.209 | 11.341 | 39.462 | 1.00 | 0.00 | −0.127 | 10.80 | 0.60 |
| ATOM | 4824 | HD2 | TYR | 295 | 13.161 | 11.137 | 38.392 | 1.00 | 0.00 | 0.127 | 0.00 | 0.00 |
| ATOM | 4825 | CE1 | TYR | 295 | 13.348 | 11.855 | 42.126 | 1.00 | 0.00 | −0.127 | 10.80 | 0.60 |
| ATOM | 4826 | HE1 | TYR | 295 | 13.407 | 12.058 | 43.195 | 1.00 | 0.00 | 0.127 | 0.00 | 0.00 |
| ATOM | 4827 | CE2 | TYR | 295 | 13.485 | 12.611 | 39.898 | 1.00 | 0.00 | −0.127 | 10.80 | 0.60 |
| ATOM | 4828 | HE2 | TYR | 295 | 13.654 | 13.409 | 39.175 | 1.00 | 0.00 | 0.127 | 0.00 | 0.00 |
| ATOM | 4829 | CZ | TYR | 295 | 13.548 | 12.877 | 41.238 | 1.00 | 0.00 | 0.026 | 7.26 | 0.60 |
| ATOM | 4830 | OH | TYR | 295 | 13.830 | 14.180 | 41.701 | 1.00 | 0.00 | −0.451 | 10.94 | −17.40 |
| ATOM | 4831 | HH | TYR | 295 | 14.795 | 14.434 | 41.446 | 1.00 | 0.00 | 0.424 | 0.00 | 0.00 |
| ATOM | 4832 | N | ASP | 296P | 10.284 | 10.397 | 41.309 | 1.00 | 0.00 | −0.650 | 9.00 | −17.40 |
| ATOM | 4833 | HN | ASP | 296P | 10.778 | 10.091 | 42.159 | 1.00 | 0.00 | 0.440 | 0.00 | 0.00 |
| ATOM | 4834 | CA | ASP | 296P | 9.488 | 11.581 | 41.338 | 1.00 | 0.00 | 0.158 | 9.40 | 4.00 |
| ATOM | 4835 | HA | ASP | 296P | 9.036 | 11.731 | 40.357 | 1.00 | 0.00 | 0.053 | 0.00 | 0.00 |
| ATOM | 4836 | C | ASP | 296P | 10.361 | 12.751 | 41.681 | 1.00 | 0.00 | 0.396 | 9.82 | 4.00 |
| ATOM | 4837 | O | ASP | 296P | 10.888 | 12.874 | 42.786 | 1.00 | 0.00 | −0.396 | 8.17 | −17.40 |
| ATOM | 4838 | CB | ASP | 296P | 8.264 | 11.481 | 42.272 | 1.00 | 0.00 | −0.336 | 12.77 | 4.00 |
| ATOM | 4839 | HB1 | ASP | 296P | 7.554 | 10.747 | 41.888 | 1.00 | 0.00 | 0.053 | 0.00 | 0.00 |
| ATOM | 4840 | HB2 | ASP | 296P | 7.762 | 12.446 | 42.342 | 1.00 | 0.00 | 0.053 | 0.00 | 0.00 |
| ATOM | 4841 | CG | ASP | 296P | 8.698 | 11.055 | 43.666 | 1.00 | 0.00 | 0.297 | 9.82 | 4.00 |
| ATOM | 4842 | OD1 | ASP | 296P | 9.913 | 10.797 | 43.867 | 1.00 | 0.00 | −0.534 | 8.17 | −18.95 |
| ATOM | 4843 | OD2 | ASP | 296P | 7.809 | 10.969 | 44.553 | 1.00 | 0.00 | −0.534 | 8.17 | −18.95 |
| ATOM | 4844 | N | LEU | 297 | 10.519 | 13.645 | 40.683 | 1.00 | 0.00 | −0.650 | 9.00 | −17.40 |
| ATOM | 4845 | HN | LEU | 297 | 10.032 | 13.446 | 39.797 | 1.00 | 0.00 | 0.440 | 0.00 | 0.00 |
| ATOM | 4846 | CA | LEU | 297 | 11.301 | 14.847 | 40.740 | 1.00 | 0.00 | 0.158 | 9.40 | 4.00 |
| ATOM | 4847 | HA | LEU | 297 | 12.305 | 14.581 | 41.067 | 1.00 | 0.00 | 0.053 | 0.00 | 0.00 |
| ATOM | 4848 | C | LEU | 297 | 10.655 | 15.772 | 41.710 | 1.00 | 0.00 | 0.396 | 9.82 | 4.00 |
| ATOM | 4849 | O | LEU | 297 | 11.194 | 16.843 | 41.977 | 1.00 | 0.00 | −0.396 | 8.17 | −17.40 |
| ATOM | 4850 | CB | LEU | 297 | 11.307 | 15.647 | 39.418 | 1.00 | 0.00 | −0.106 | 12.77 | 4.00 |
| ATOM | 4851 | HB1 | LEU | 297 | 11.713 | 16.634 | 39.635 | 1.00 | 0.00 | 0.053 | 0.00 | 0.00 |
| ATOM | 4852 | HB2 | LEU | 297 | 10.276 | 15.708 | 39.069 | 1.00 | 0.00 | 0.053 | 0.00 | 0.00 |
| ATOM | 4853 | CG | LEU | 297 | 12.132 | 15.086 | 38.242 | 1.00 | 0.00 | −0.053 | 9.40 | 4.00 |
| ATOM | 4854 | HG | LEU | 297 | 11.921 | 15.624 | 37.318 | 1.00 | 0.00 | 0.053 | 0.00 | 0.00 |
| ATOM | 4855 | CD1 | LEU | 297 | 13.641 | 15.201 | 38.513 | 1.00 | 0.00 | −0.159 | 16.15 | 4.00 |
| ATOM | 4856 | HD1 | LEU | 297 | 14.194 | 14.797 | 37.665 | 1.00 | 0.00 | 0.053 | 0.00 | 0.00 |
| ATOM | 4857 | HD1 | LEU | 297 | 13.906 | 16.248 | 38.653 | 1.00 | 0.00 | 0.053 | 0.00 | 0.00 |
| ATOM | 4858 | HD1 | LEU | 297 | 13.893 | 14.638 | 39.412 | 1.00 | 0.00 | 0.053 | 0.00 | 0.00 |
| ATOM | 4859 | CD2 | LEU | 297 | 11.679 | 13.675 | 37.832 | 1.00 | 0.00 | −0.159 | 16.15 | 4.00 |
| ATOM | 4860 | HD2 | LEU | 297 | 12.290 | 13.323 | 37.000 | 1.00 | 0.00 | 0.053 | 0.00 | 0.00 |
| ATOM | 4861 | HD2 | LEU | 297 | 11.792 | 12.996 | 38.677 | 1.00 | 0.00 | 0.053 | 0.00 | 0.00 |
| ATOM | 4862 | HD2 | LEU | 297 | 10.632 | 13.703 | 37.527 | 1.00 | 0.00 | 0.053 | 0.00 | 0.00 |
| ATOM | 4863 | N | SER | 298 | 9.472 | 15.382 | 42.228 | 1.00 | 0.00 | −0.650 | 9.00 | −17.40 |
| ATOM | 4864 | HN | SER | 298 | 9.126 | 14.441 | 41.988 | 1.00 | 0.00 | 0.440 | 0.00 | 0.00 |
| ATOM | 4865 | CA | SER | 298 | 8.665 | 16.198 | 43.093 | 1.00 | 0.00 | 0.158 | 9.40 | 4.00 |
| ATOM | 4866 | HA | SER | 298 | 8.134 | 16.961 | 42.523 | 1.00 | 0.00 | 0.053 | 0.00 | 0.00 |
| ATOM | 4867 | C | SER | 298 | 9.507 | 16.886 | 44.113 | 1.00 | 0.00 | 0.396 | 9.82 | 4.00 |
| ATOM | 4868 | O | SER | 298 | 10.597 | 16.432 | 44.457 | 1.00 | 0.00 | −0.396 | 8.17 | −17.40 |
| ATOM | 4869 | CB | SER | 298 | 7.571 | 15.412 | 43.838 | 1.00 | 0.00 | 0.007 | 12.77 | 4.00 |
| ATOM | 4870 | HB1 | SER | 298 | 8.023 | 14.638 | 44.458 | 1.00 | 0.00 | 0.053 | 0.00 | 0.00 |
| ATOM | 4871 | HB2 | SER | 298 | 6.896 | 14.940 | 43.123 | 1.00 | 0.00 | 0.053 | 0.00 | 0.00 |
| ATOM | 4872 | OG | SER | 298 | 6.822 | 16.287 | 44.668 | 1.00 | 0.00 | −0.537 | 11.04 | −17.40 |
| ATOM | 4873 | HG | SER | 298 | 7.239 | 17.228 | 44.640 | 1.00 | 0.00 | 0.424 | 0.00 | 0.00 |
| ATOM | 4874 | N | APC | 299 | 9.017 | 18.045 | 44.592 | 1.00 | 0.00 | −0.650 | 9.00 | −17.40 |
| ATOM | 4875 | HN | APC | 299 | 8.109 | 18.391 | 44.250 | 1.00 | 0.00 | 0.440 | 0.00 | 0.00 |
| ATOM | 4876 | CA | APC | 299 | 9.739 | 18.802 | 45.568 | 1.00 | 0.00 | −0.072 | 9.40 | 4.00 |
| ATOM | 4877 | HA | APC | 299 | 10.795 | 18.733 | 45.309 | 1.00 | 0.00 | 0.053 | 0.00 | 0.00 |
| ATOM | 4878 | C | APC | 299 | 9.449 | 18.176 | 46.924 | 1.00 | 0.00 | 0.297 | 9.82 | 4.00 |
| ATOM | 4879 | O | APC | 299 | 8.563 | 17.282 | 46.985 | 1.00 | 0.00 | −0.534 | 8.17 | −17.40 |
| ATOM | 4880 | OXT | APC | 299 | 10.105 | 18.587 | 47.918 | 1.00 | 0.00 | −0.534 | 0.00 | 0.00 |
| ATOM | 4881 | CB | APC | 299 | 9.312 | 20.279 | 45.642 | 1.00 | 0.00 | −0.336 | 0.00 | 0.00 |
| ATOM | 4882 | HB1 | APC | 299 | 9.434 | 20.722 | 44.653 | 1.00 | 0.00 | 0.053 | 0.00 | 0.00 |
| ATOM | 4883 | HB2 | APC | 299 | 9.947 | 20.785 | 46.369 | 1.00 | 0.00 | 0.053 | 0.00 | 0.00 |
| ATOM | 4884 | CG | APC | 299 | 7.854 | 20.337 | 46.076 | 1.00 | 0.00 | 0.297 | 0.00 | 0.00 |
| ATOM | 4885 | OD1 | APC | 299 | 7.148 | 19.302 | 45.937 | 1.00 | 0.00 | −0.534 | 0.00 | 0.00 |
| ATOM | 4886 | OD2 | APC | 299 | 7.426 | 21.421 | 46.556 | 1.00 | 0.00 | −0.534 | 0.00 | 0.00 |
| ATOM | 4887 | MN | MET | 300 | 1.840 | 17.834 | 13.469 | 1.00 | 0.00 | 0.600 | 12.00 | 4.00 |

REMARK Solvation parameters added by “addsol”

TABLE 4
Core 2L mouse model

| REMARK Produced by MODELLER: 14 Feb. 00 09:59:31 | | | | | | | | | | | 1 |
|---|---|---|---|---|---|---|---|---|---|---|---|
| REMARK MODELLER OBJECTIVE FUNCTION: 4193.6301 | | | | | | | | | | | |
| ATOM | 1 | N | LEU | 1 | −19.427 | −7.161 | 11.220 | 1.00 | 0.53 | 1SG | 2 |
| ATOM | 2 | CA | LEU | 1 | −18.305 | −6.448 | 10.569 | 1.00 | 0.53 | 1SG | 3 |
| ATOM | 3 | CB | LEU | 1 | −18.415 | −4.937 | 10.828 | 1.00 | 0.53 | 1SG | 4 |
| ATOM | 4 | CG | LEU | 1 | −19.689 | −4.304 | 10.238 | 1.00 | 0.53 | 1SG | 5 |
| ATOM | 5 | CD2 | LEU | 1 | −19.844 | −4.644 | 8.747 | 1.00 | 0.53 | 1SG | 6 |
| ATOM | 6 | CD1 | LEU | 1 | −19.746 | −2.794 | 10.518 | 1.00 | 0.53 | 1SG | 7 |
| ATOM | 7 | C | LEU | 1 | −17.017 | −6.929 | 11.136 | 1.00 | 0.53 | 1SG | 8 |
| ATOM | 8 | O | LEU | 1 | −16.764 | −8.129 | 11.231 | 1.00 | 0.53 | 1SG | 9 |
| ATOM | 9 | N | ALA | 2 | −16.162 | −5.970 | 11.522 | 1.00 | 0.42 | 1SG | 10 |
| ATOM | 10 | CA | ALA | 2 | −14.877 | −6.262 | 12.072 | 1.00 | 0.42 | 1SG | 11 |
| ATOM | 11 | CB | ALA | 2 | −13.863 | −6.763 | 11.031 | 1.00 | 0.42 | 1SG | 12 |
| ATOM | 12 | C | ALA | 2 | −14.388 | −4.949 | 12.569 | 1.00 | 0.42 | 1SG | 13 |
| ATOM | 13 | O | ALA | 2 | −15.127 | −4.211 | 13.219 | 1.00 | 0.42 | 1SG | 14 |
| ATOM | 14 | N | GLY | 3 | −13.108 | −4.633 | 12.315 | 1.00 | 0.29 | 1SG | 15 |
| ATOM | 15 | CA | GLY | 3 | −12.657 | −3.339 | 12.717 | 1.00 | 0.29 | 1SG | 16 |
| ATOM | 16 | C | GLY | 3 | −12.084 | −2.680 | 11.508 | 1.00 | 0.29 | 1SG | 17 |
| ATOM | 17 | O | GLY | 3 | −10.974 | −2.996 | 11.083 | 1.00 | 0.29 | 1SG | 18 |
| ATOM | 18 | N | ASP | 4 | −12.840 | −1.732 | 10.924 | 1.00 | 0.21 | 1SG | 19 |
| ATOM | 19 | CA | ASP | 4 | −12.364 | −1.017 | 9.779 | 1.00 | 0.21 | 1SG | 20 |
| ATOM | 20 | CB | ASP | 4 | −13.382 | −0.974 | 8.628 | 1.00 | 0.21 | 1SG | 21 |
| ATOM | 21 | CG | ASP | 4 | −13.663 | −2.393 | 8.171 | 1.00 | 0.21 | 1SG | 22 |
| ATOM | 22 | OD1 | ASP | 4 | −13.004 | −3.325 | 8.705 | 1.00 | 0.21 | 1SG | 23 |
| ATOM | 23 | OD2 | ASP | 4 | −14.538 | −2.568 | 7.281 | 1.00 | 0.21 | 1SG | 24 |
| ATOM | 24 | C | ASP | 4 | −12.242 | 0.396 | 10.236 | 1.00 | 0.21 | 1SG | 25 |
| ATOM | 25 | O | ASP | 4 | −13.248 | 1.083 | 10.404 | 1.00 | 0.21 | 1SG | 26 |
| ATOM | 26 | N | ASP | 5 | −11.005 | 0.876 | 10.440 | 1.00 | 0.32 | 1SG | 27 |
| ATOM | 27 | CA | ASP | 5 | −10.857 | 2.218 | 10.911 | 1.00 | 0.32 | 1SG | 28 |
| ATOM | 28 | CB | ASP | 5 | −9.876 | 2.335 | 12.087 | 1.00 | 0.32 | 1SG | 29 |
| ATOM | 29 | CG | ASP | 5 | −8.527 | 1.819 | 11.607 | 1.00 | 0.32 | 1SG | 30 |
| ATOM | 30 | OD1 | ASP | 5 | −8.474 | 0.638 | 11.167 | 1.00 | 0.32 | 1SG | 31 |
| ATOM | 31 | OD2 | ASP | 5 | −7.532 | 2.587 | 11.680 | 1.00 | 0.32 | 1SG | 32 |
| ATOM | 32 | C | ASP | 5 | −10.316 | 3.038 | 9.790 | 1.00 | 0.32 | 1SG | 33 |
| ATOM | 33 | O | ASP | 5 | −9.349 | 2.666 | 9.130 | 1.00 | 0.32 | 1SG | 34 |
| ATOM | 34 | N | PRO | 6 | −10.970 | 4.133 | 9.539 | 1.00 | 0.45 | 1SG | 35 |
| ATOM | 35 | CA | PRO | 6 | −10.505 | 5.025 | 8.516 | 1.00 | 0.45 | 1SG | 36 |
| ATOM | 36 | CD | PRO | 6 | −12.416 | 4.166 | 9.663 | 1.00 | 0.45 | 1SG | 37 |
| ATOM | 37 | CB | PRO | 6 | −11.725 | 5.823 | 8.045 | 1.00 | 0.45 | 1SG | 38 |
| ATOM | 38 | CG | PRO | 6 | −12.812 | 5.537 | 9.098 | 1.00 | 0.45 | 1SG | 39 |
| ATOM | 39 | C | PRO | 6 | −9.360 | 5.857 | 9.000 | 1.00 | 0.45 | 1SG | 40 |
| ATOM | 40 | O | PRO | 6 | −9.355 | 6.237 | 10.172 | 1.00 | 0.45 | 1SG | 41 |
| ATOM | 41 | N | TYR | 7 | −8.390 | 6.157 | 8.111 | 1.00 | 0.37 | 1SG | 42 |
| ATOM | 42 | CA | TYR | 7 | −7.269 | 6.968 | 8.484 | 1.00 | 0.37 | 1SG | 43 |
| ATOM | 43 | CB | TYR | 7 | −5.912 | 6.495 | 7.918 | 1.00 | 0.37 | 1SG | 44 |
| ATOM | 44 | CG | TYR | 7 | −5.387 | 5.275 | 8.595 | 1.00 | 0.37 | 1SG | 45 |
| ATOM | 45 | CD1 | TYR | 7 | −5.891 | 4.022 | 8.340 | 1.00 | 0.37 | 1SG | 46 |
| ATOM | 46 | CD2 | TYR | 7 | −4.340 | 5.400 | 9.476 | 1.00 | 0.37 | 1SG | 47 |
| ATOM | 47 | CE1 | TYR | 7 | −5.365 | 2.919 | 8.976 | 1.00 | 0.37 | 1SG | 48 |
| ATOM | 48 | CE2 | TYR | 7 | −3.810 | 4.308 | 10.115 | 1.00 | 0.37 | 1SG | 49 |
| ATOM | 49 | CZ | TYR | 7 | −4.326 | 3.062 | 9.865 | 1.00 | 0.37 | 1SG | 50 |
| ATOM | 50 | OH | TYR | 7 | −3.782 | 1.937 | 10.520 | 1.00 | 0.37 | 1SG | 51 |
| ATOM | 51 | C | TYR | 7 | −7.468 | 8.296 | 7.842 | 1.00 | 0.37 | 1SG | 52 |
| ATOM | 52 | O | TYR | 7 | −7.844 | 8.380 | 6.675 | 1.00 | 0.37 | 1SG | 53 |
| ATOM | 53 | N | SER | 8 | −7.233 | 9.384 | 8.591 | 1.00 | 0.28 | 1SG | 54 |
| ATOM | 54 | CA | SER | 8 | −7.343 | 10.663 | 7.969 | 1.00 | 0.28 | 1SG | 55 |
| ATOM | 55 | CB | SER | 8 | −8.300 | 11.623 | 8.692 | 1.00 | 0.28 | 1SG | 56 |
| ATOM | 56 | OG | SER | 8 | −7.894 | 11.791 | 10.040 | 1.00 | 0.28 | 1SG | 57 |
| ATOM | 57 | C | SER | 8 | −5.973 | 11.245 | 7.992 | 1.00 | 0.28 | 1SG | 58 |
| ATOM | 58 | O | SER | 8 | −5.366 | 11.390 | 9.052 | 1.00 | 0.28 | 1SG | 59 |
| ATOM | 59 | N | ASN | 9 | −5.431 | 11.572 | 6.805 | 1.00 | 0.28 | 1SG | 60 |
| ATOM | 60 | CA | ASN | 9 | −4.114 | 12.126 | 6.802 | 1.00 | 0.28 | 1SG | 61 |
| ATOM | 61 | CB | ASN | 9 | −3.125 | 11.449 | 5.842 | 1.00 | 0.28 | 1SG | 62 |
| ATOM | 62 | CG | ASN | 9 | −1.763 | 12.018 | 6.210 | 1.00 | 0.28 | 1SG | 63 |
| ATOM | 63 | OD1 | ASN | 9 | −1.606 | 12.553 | 7.304 | 1.00 | 0.28 | 1SG | 64 |
| ATOM | 64 | ND2 | ASN | 9 | −0.763 | 11.917 | 5.297 | 1.00 | 0.28 | 1SG | 65 |
| ATOM | 65 | C | ASN | 9 | −4.228 | 13.540 | 6.352 | 1.00 | 0.28 | 1SG | 66 |
| ATOM | 66 | O | ASN | 9 | −5.057 | 13.870 | 5.506 | 1.00 | 0.28 | 1SG | 67 |
| ATOM | 67 | N | VAL | 10 | −3.393 | 14.425 | 6.922 | 1.00 | 0.36 | 1SG | 68 |
| ATOM | 68 | CA | VAL | 10 | −3.450 | 15.795 | 6.515 | 1.00 | 0.36 | 1SG | 69 |
| ATOM | 69 | CB | VAL | 10 | −3.572 | 16.729 | 7.661 | 1.00 | 0.36 | 1SG | 70 |
| ATOM | 70 | CG1 | VAL | 10 | −4.960 | 16.483 | 8.276 | 1.00 | 0.36 | 1SG | 71 |
| ATOM | 71 | CG2 | VAL | 10 | −2.398 | 16.453 | 8.617 | 1.00 | 0.36 | 1SG | 72 |
| ATOM | 72 | C | VAL | 10 | −2.199 | 16.103 | 5.771 | 1.00 | 0.36 | 1SG | 73 |
| ATOM | 73 | O | VAL | 10 | −1.121 | 15.615 | 6.108 | 1.00 | 0.36 | 1SG | 74 |
| ATOM | 74 | N | ASN | 11 | −2.326 | 16.912 | 4.700 | 1.00 | 0.45 | 1SG | 75 |
| ATOM | 75 | CA | ASN | 11 | −1.195 | 17.134 | 3.850 | 1.00 | 0.45 | 1SG | 76 |
| ATOM | 76 | CB | ASN | 11 | −1.278 | 16.250 | 2.593 | 1.00 | 0.45 | 1SG | 77 |
| ATOM | 77 | CG | ASN | 11 | −1.890 | 14.893 | 2.958 | 1.00 | 0.45 | 1SG | 78 |
| ATOM | 78 | OD1 | ASN | 11 | −1.567 | 14.202 | 3.922 | 1.00 | 0.45 | 1SG | 79 |
| ATOM | 79 | ND2 | ASN | 11 | −2.877 | 14.476 | 2.125 | 1.00 | 0.45 | 1SG | 80 |
| ATOM | 80 | C | ASN | 11 | −1.317 | 18.523 | 3.301 | 1.00 | 0.45 | 1SG | 81 |
| ATOM | 81 | O | ASN | 11 | −2.353 | 19.170 | 3.449 | 1.00 | 0.45 | 1SG | 82 |
| ATOM | 82 | N | CYS | 12 | −0.241 | 19.025 | 2.654 | 1.00 | 0.57 | 1SG | 83 |
| ATOM | 83 | CA | CYS | 12 | −0.354 | 20.284 | 1.971 | 1.00 | 0.57 | 1SG | 84 |
| ATOM | 84 | CB | CYS | 12 | −0.558 | 21.547 | 2.818 | 1.00 | 0.57 | 1SG | 85 |
| ATOM | 85 | SG | CYS | 12 | −1.361 | 22.865 | 1.833 | 1.00 | 0.57 | 1SG | 86 |
| ATOM | 86 | C | CYS | 12 | 0.912 | 20.538 | 1.221 | 1.00 | 0.57 | 1SG | 87 |
| ATOM | 87 | O | CYS | 12 | 1.290 | 19.793 | 0.320 | 1.00 | 0.57 | 1SG | 88 |
| ATOM | 88 | N | THR | 13 | 1.565 | 21.659 | 1.587 | 1.00 | 0.62 | 1SG | 89 |
| ATOM | 89 | CA | THR | 13 | 2.708 | 22.231 | 0.940 | 1.00 | 0.62 | 1SG | 90 |
| ATOM | 90 | CB | THR | 13 | 3.088 | 23.556 | 1.534 | 1.00 | 0.62 | 1SG | 91 |
| ATOM | 91 | OG1 | THR | 13 | 4.136 | 24.152 | 0.784 | 1.00 | 0.62 | 1SG | 92 |
| ATOM | 92 | CG2 | THR | 13 | 3.526 | 23.337 | 2.993 | 1.00 | 0.62 | 1SG | 93 |
| ATOM | 93 | C | THR | 13 | 3.903 | 21.346 | 1.025 | 1.00 | 0.62 | 1SG | 94 |
| ATOM | 94 | O | THR | 13 | 4.656 | 21.260 | 0.057 | 1.00 | 0.62 | 1SG | 95 |
| ATOM | 95 | N | LYS | 14 | 4.106 | 20.676 | 2.177 | 1.00 | 0.50 | 1SG | 96 |
| ATOM | 96 | CA | LYS | 14 | 5.288 | 19.884 | 2.379 | 1.00 | 0.50 | 1SG | 97 |
| ATOM | 97 | CB | LYS | 14 | 5.243 | 19.051 | 3.674 | 1.00 | 0.50 | 1SG | 98 |
| ATOM | 98 | CG | LYS | 14 | 5.231 | 19.913 | 4.937 | 1.00 | 0.50 | 1SG | 99 |
| ATOM | 99 | CD | LYS | 14 | 4.806 | 19.169 | 6.205 | 1.00 | 0.50 | 1SG | 100 |
| ATOM | 100 | CE | LYS | 14 | 4.802 | 20.051 | 7.459 | 1.00 | 0.50 | 1SG | 101 |
| ATOM | 101 | NZ | LYS | 14 | 3.664 | 20.999 | 7.421 | 1.00 | 0.50 | 1SG | 102 |
| ATOM | 102 | C | LYS | 14 | 5.443 | 18.937 | 1.239 | 1.00 | 0.50 | 1SG | 103 |
| ATOM | 103 | O | LYS | 14 | 4.728 | 17.943 | 1.131 | 1.00 | 0.50 | 1SG | 104 |
| ATOM | 104 | N | ILE | 15 | 6.400 | 19.251 | 0.346 | 1.00 | 0.37 | 1SG | 105 |
| ATOM | 105 | CA | ILE | 15 | 6.649 | 18.427 | −0.794 | 1.00 | 0.37 | 1SG | 106 |
| ATOM | 106 | CB | ILE | 15 | 6.248 | 19.060 | −2.096 | 1.00 | 0.37 | 1SG | 107 |
| ATOM | 107 | CG2 | ILE | 15 | 4.742 | 19.357 | −2.026 | 1.00 | 0.37 | 1SG | 108 |
| ATOM | 108 | CD1 | ILE | 15 | 7.110 | 20.301 | −2.399 | 1.00 | 0.37 | 1SG | 109 |
| ATOM | 109 | CD1 | ILE | 15 | 6.996 | 21.419 | −1.365 | 1.00 | 0.37 | 1SG | 110 |
| ATOM | 110 | C | ILE | 15 | 8.126 | 18.248 | −0.859 | 1.00 | 0.37 | 1SG | 111 |
| ATOM | 111 | O | ILE | 15 | 8.883 | 19.011 | −0.257 | 1.00 | 0.37 | 1SG | 112 |
| ATOM | 112 | N | LEU | 16 | 8.577 | 17.211 | −1.589 | 1.00 | 0.25 | 1SG | 113 |
| ATOM | 113 | CA | LEU | 16 | 9.984 | 17.006 | −1.728 | 1.00 | 0.25 | 1SG | 114 |
| ATOM | 114 | CB | LEU | 16 | 10.376 | 15.539 | −1.970 | 1.00 | 0.25 | 1SG | 115 |
| ATOM | 115 | CG | LEU | 16 | 11.896 | 15.349 | −2.130 | 1.00 | 0.25 | 1SG | 116 |
| ATOM | 116 | CD2 | LEU | 16 | 12.229 | 14.047 | −2.863 | 1.00 | 0.25 | 1SG | 117 |
| ATOM | 117 | CD1 | LEU | 16 | 12.628 | 15.511 | −0.783 | 1.00 | 0.25 | 1SG | 118 |
| ATOM | 118 | C | LEU | 16 | 10.372 | 17.737 | −2.970 | 1.00 | 0.25 | 1SG | 119 |
| ATOM | 119 | O | LEU | 16 | 9.571 | 17.863 | −3.897 | 1.00 | 0.25 | 1SG | 120 |
| ATOM | 120 | N | GLN | 17 | 11.610 | 18.257 | −3.015 | 1.00 | 0.15 | 1SG | 121 |
| ATOM | 121 | CA | GLN | 17 | 12.006 | 18.960 | −4.196 | 1.00 | 0.15 | 1SG | 122 |
| ATOM | 122 | CB | GLN | 17 | 13.443 | 19.506 | −4.158 | 1.00 | 0.15 | 1SG | 123 |
| ATOM | 123 | CG | GLN | 17 | 13.832 | 20.262 | −5.433 | 1.00 | 0.15 | 1SG | 124 |
| ATOM | 124 | CD | GLN | 17 | 15.267 | 20.748 | −5.284 | 1.00 | 0.15 | 1SG | 125 |
| ATOM | 125 | OE1 | GLN | 17 | 15.769 | 21.494 | −6.123 | 1.00 | 0.15 | 1SG | 126 |
| ATOM | 126 | NE2 | GLN | 17 | 15.946 | 20.318 | −4.187 | 1.00 | 0.15 | 1SG | 127 |
| ATOM | 127 | C | GLN | 17 | 11.914 | 17.987 | −5.315 | 1.00 | 0.15 | 1SG | 128 |
| ATOM | 128 | O | GLN | 17 | 12.441 | 16.880 | −5.234 | 1.00 | 0.15 | 1SG | 129 |
| ATOM | 129 | N | GLY | 18 | 11.256 | 18.394 | −6.413 | 1.00 | 0.11 | 1SG | 130 |
| ATOM | 130 | CA | GLY | 18 | 11.067 | 17.493 | −7.506 | 1.00 | 0.11 | 1SG | 131 |
| ATOM | 131 | C | GLY | 18 | 9.616 | 17.146 | −7.551 | 1.00 | 0.11 | 1SG | 132 |
| ATOM | 132 | O | GLY | 18 | 8.818 | 17.659 | −6.770 | 1.00 | 0.11 | 1SG | 133 |
| ATOM | 133 | N | ASP | 19 | 9.240 | 16.273 | −8.503 | 1.00 | 0.22 | 1SG | 134 |
| ATOM | 134 | CA | ASP | 19 | 7.870 | 15.892 | −8.670 | 1.00 | 0.22 | 1SG | 135 |
| ATOM | 135 | CB | ASP | 19 | 7.644 | 14.943 | −9.859 | 1.00 | 0.22 | 1SG | 136 |
| ATOM | 136 | CG | ASP | 19 | 7.784 | 15.761 | −11.135 | 1.00 | 0.22 | 1SG | 137 |
| ATOM | 137 | OD1 | ASP | 19 | 7.873 | 17.013 | −11.026 | 1.00 | 0.22 | 1SG | 138 |
| ATOM | 138 | OD2 | ASP | 19 | 7.796 | 15.147 | −12.235 | 1.00 | 0.22 | 1SG | 139 |
| ATOM | 139 | C | ASP | 19 | 7.394 | 15.200 | −7.434 | 1.00 | 0.22 | 1SG | 140 |
| ATOM | 140 | O | ASP | 19 | 6.281 | 15.457 | −6.979 | 1.00 | 0.22 | 1SG | 141 |
| ATOM | 141 | N | PRO | 20 | 8.187 | 14.346 | −6.851 | 1.00 | 0.45 | 1SG | 142 |
| ATOM | 142 | CA | PRO | 20 | 7.719 | 13.678 | −5.675 | 1.00 | 0.45 | 1SG | 143 |
| ATOM | 143 | CD | PRO | 20 | 9.098 | 13.509 | −7.616 | 1.00 | 0.45 | 1SG | 144 |
| ATOM | 144 | CB | PRO | 20 | 8.717 | 12.556 | −5.405 | 1.00 | 0.45 | 1SG | 145 |
| ATOM | 145 | CG | PRO | 20 | 9.228 | 12.201 | −6.815 | 1.00 | 0.45 | 1SG | 146 |
| ATOM | 146 | C | PRO | 20 | 7.554 | 14.659 | −4.573 | 1.00 | 0.45 | 1SG | 147 |
| ATOM | 147 | O | PRO | 20 | 8.209 | 15.701 | −4.587 | 1.00 | 0.45 | 1SG | 148 |
| ATOM | 148 | N | GLU | 21 | 6.656 | 14.348 | −3.623 | 1.00 | 0.51 | 1SG | 149 |
| ATOM | 149 | CA | GLU | 21 | 6.390 | 15.254 | −2.557 | 1.00 | 0.51 | 1SG | 150 |
| ATOM | 150 | CB | GLU | 21 | 4.883 | 15.468 | −2.340 | 1.00 | 0.51 | 1SG | 151 |
| ATOM | 151 | CG | GLU | 21 | 4.100 | 15.798 | −3.620 | 1.00 | 0.51 | 1SG | 152 |
| ATOM | 152 | CD | GLU | 21 | 4.589 | 17.100 | −4.242 | 1.00 | 0.51 | 1SG | 153 |
| ATOM | 153 | OE1 | GLU | 21 | 5.820 | 17.365 | −4.210 | 1.00 | 0.51 | 1SG | 154 |
| ATOM | 154 | OE2 | GLU | 21 | 3.726 | 17.847 | −4.775 | 1.00 | 0.51 | 1SG | 155 |
| ATOM | 155 | C | GLU | 21 | 6.906 | 14.573 | −1.337 | 1.00 | 0.51 | 1SG | 156 |
| ATOM | 156 | O | GLU | 21 | 7.130 | 13.365 | −1.344 | 1.00 | 0.51 | 1SG | 157 |
| ATOM | 157 | N | GLU | 22 | 7.132 | 15.348 | −0.266 | 1.00 | 0.41 | 1SG | 158 |
| ATOM | 158 | CA | GLU | 22 | 7.587 | 14.839 | 0.994 | 1.00 | 0.41 | 1SG | 159 |
| ATOM | 159 | CB | GLU | 22 | 7.737 | 15.966 | 2.027 | 1.00 | 0.41 | 1SG | 160 |
| ATOM | 160 | CG | GLU | 22 | 8.215 | 15.508 | 3.403 | 1.00 | 0.41 | 1SG | 161 |
| ATOM | 161 | CD | GLU | 22 | 8.160 | 16.712 | 4.336 | 1.00 | 0.41 | 1SG | 162 |
| ATOM | 162 | OE1 | GLU | 22 | 8.898 | 17.700 | 4.078 | 1.00 | 0.41 | 1SG | 163 |
| ATOM | 163 | OE2 | GLU | 22 | 7.373 | 16.660 | 5.318 | 1.00 | 0.41 | 1SG | 164 |
| ATOM | 164 | C | GLU | 22 | 6.502 | 13.938 | 1.489 | 1.00 | 0.41 | 1SG | 165 |
| ATOM | 165 | O | GLU | 22 | 6.746 | 12.954 | 2.182 | 1.00 | 0.41 | 1SG | 166 |
| ATOM | 166 | N | ILE | 23 | 5.268 | 14.304 | 1.117 | 1.00 | 0.36 | 1SG | 167 |
| ATOM | 167 | CA | ILE | 23 | 3.999 | 13.698 | 1.408 | 1.00 | 0.36 | 1SG | 168 |
| ATOM | 168 | CB | ILE | 23 | 2.946 | 14.434 | 0.618 | 1.00 | 0.36 | 1SG | 169 |
| ATOM | 169 | CG2 | ILE | 23 | 1.787 | 13.497 | 0.242 | 1.00 | 0.36 | 1SG | 170 |
| ATOM | 170 | CD1 | ILE | 23 | 2.603 | 15.780 | 1.276 | 1.00 | 0.36 | 1SG | 171 |
| ATOM | 171 | CD1 | ILE | 23 | 1.745 | 16.690 | 0.399 | 1.00 | 0.36 | 1SG | 172 |
| ATOM | 172 | C | ILE | 23 | 3.970 | 12.294 | 0.895 | 1.00 | 0.36 | 1SG | 173 |
| ATOM | 173 | O | ILE | 23 | 3.312 | 11.432 | 1.466 | 1.00 | 0.36 | 1SG | 174 |
| ATOM | 174 | N | GLN | 24 | 4.613 | 12.054 | −0.250 | 1.00 | 0.50 | 1SG | 175 |
| ATOM | 175 | CA | GLN | 24 | 4.576 | 10.793 | −0.934 | 1.00 | 0.50 | 1SG | 176 |
| ATOM | 176 | CB | GLN | 24 | 5.079 | 10.957 | −2.376 | 1.00 | 0.50 | 1SG | 177 |
| ATOM | 177 | CG | GLN | 24 | 5.079 | 9.676 | −3.202 | 1.00 | 0.50 | 1SG | 178 |
| ATOM | 178 | CD | GLN | 24 | 5.600 | 10.042 | −4.585 | 1.00 | 0.50 | 1SG | 179 |
| ATOM | 179 | OE1 | GLN | 24 | 5.614 | 11.215 | −4.957 | 1.00 | 0.50 | 1SG | 180 |
| ATOM | 180 | NE2 | GLN | 24 | 6.031 | 9.020 | −5.371 | 1.00 | 0.50 | 1SG | 181 |
| ATOM | 181 | C | GLN | 24 | 5.369 | 9.691 | −0.287 | 1.00 | 0.50 | 1SG | 182 |
| ATOM | 182 | O | GLN | 24 | 4.949 | 8.537 | −0.273 | 1.00 | 0.50 | 1SG | 183 |
| ATOM | 183 | N | LYS | 25 | 6.524 | 10.033 | 0.295 | 1.00 | 0.63 | 1SG | 184 |
| ATOM | 184 | CA | LYS | 25 | 7.566 | 9.130 | 0.713 | 1.00 | 0.63 | 1SG | 185 |
| ATOM | 185 | CB | LYS | 25 | 8.721 | 9.912 | 1.360 | 1.00 | 0.63 | 1SG | 186 |
| ATOM | 186 | CG | LYS | 25 | 9.753 | 9.049 | 2.087 | 1.00 | 0.63 | 1SG | 187 |
| ATOM | 187 | CD | LYS | 25 | 10.963 | 9.845 | 2.583 | 1.00 | 0.63 | 1SG | 188 |
| ATOM | 188 | CE | LYS | 25 | 11.856 | 9.070 | 3.555 | 1.00 | 0.63 | 1SG | 189 |
| ATOM | 189 | NZ | LYS | 25 | 12.635 | 8.042 | 2.831 | 1.00 | 0.63 | 1SG | 190 |
| ATOM | 190 | C | LYS | 25 | 7.182 | 8.136 | 1.740 | 1.00 | 0.63 | 1SG | 191 |
| ATOM | 191 | O | LYS | 25 | 7.672 | 7.007 | 1.732 | 1.00 | 0.63 | 1SG | 192 |
| ATOM | 192 | N | VAL | 26 | 6.209 | 8.594 | 2.517 | 1.00 | 0.72 | 1SG | 193 |
| ATOM | 193 | CA | VAL | 26 | 5.931 | 8.426 | 3.890 | 1.00 | 0.72 | 1SG | 194 |
| ATOM | 194 | CB | VAL | 26 | 5.342 | 9.718 | 4.339 | 1.00 | 0.72 | 1SG | 195 |
| ATOM | 195 | CG1 | VAL | 26 | 6.329 | 10.841 | 3.986 | 1.00 | 0.72 | 1SG | 196 |
| ATOM | 196 | CG2 | VAL | 26 | 4.014 | 9.897 | 3.584 | 1.00 | 0.72 | 1SG | 197 |
| ATOM | 197 | C | VAL | 26 | 4.988 | 7.410 | 4.372 | 1.00 | 0.72 | 1SG | 198 |
| ATOM | 198 | O | VAL | 26 | 4.889 | 6.279 | 3.912 | 1.00 | 0.72 | 1SG | 199 |
| ATOM | 199 | N | LYS | 27 | 4.345 | 7.863 | 5.468 | 1.00 | 0.83 | 1SG | 200 |
| ATOM | 200 | CA | LYS | 27 | 3.331 | 7.177 | 6.170 | 1.00 | 0.83 | 1SG | 201 |
| ATOM | 201 | CB | LYS | 27 | 2.792 | 7.972 | 7.378 | 1.00 | 0.83 | 1SG | 202 |
| ATOM | 202 | CG | LYS | 27 | 2.355 | 9.408 | 7.072 | 1.00 | 0.83 | 1SG | 203 |
| ATOM | 203 | CD | LYS | 27 | 1.078 | 9.504 | 6.238 | 1.00 | 0.83 | 1SG | 204 |
| ATOM | 204 | CE | LYS | 27 | −0.159 | 8.955 | 6.948 | 1.00 | 0.83 | 1SG | 205 |
| ATOM | 205 | NZ | LYS | 27 | −1.337 | 9.061 | 6.060 | 1.00 | 0.83 | 1SG | 206 |
| ATOM | 206 | C | LYS | 27 | 2.257 | 6.969 | 5.173 | 1.00 | 0.83 | 1SG | 207 |
| ATOM | 207 | O | LYS | 27 | 1.496 | 6.013 | 5.264 | 1.00 | 0.83 | 1SG | 208 |
| ATOM | 208 | N | LEU | 28 | 2.139 | 7.878 | 4.194 | 1.00 | 0.63 | 1SG | 209 |
| ATOM | 209 | CA | LEU | 28 | 1.141 | 7.651 | 3.201 | 1.00 | 0.63 | 1SG | 210 |
| ATOM | 210 | CB | LEU | 28 | 1.104 | 8.768 | 2.143 | 1.00 | 0.63 | 1SG | 211 |
| ATOM | 211 | CG | LEU | 28 | 0.147 | 8.494 | 0.966 | 1.00 | 0.63 | 1SG | 212 |
| ATOM | 212 | CD2 | LEU | 28 | −1.185 | 7.922 | 1.473 | 1.00 | 0.63 | 1SG | 213 |
| ATOM | 213 | CD1 | LEU | 28 | 0.781 | 7.582 | −0.099 | 1.00 | 0.63 | 1SG | 214 |
| ATOM | 214 | C | LEU | 28 | 1.457 | 6.379 | 2.499 | 1.00 | 0.63 | 1SG | 215 |
| ATOM | 215 | O | LEU | 28 | 0.639 | 5.462 | 2.461 | 1.00 | 0.63 | 1SG | 216 |
| ATOM | 216 | N | GLU | 29 | 2.674 | 6.262 | 1.947 | 1.00 | 0.27 | 1SG | 217 |
| ATOM | 217 | CA | GLU | 29 | 2.951 | 5.071 | 1.209 | 1.00 | 0.27 | 1SG | 218 |
| ATOM | 218 | CB | GLU | 29 | 4.319 | 5.075 | 0.507 | 1.00 | 0.27 | 1SG | 219 |
| ATOM | 219 | CG | GLU | 29 | 5.512 | 5.078 | 1.465 | 1.00 | 0.27 | 1SG | 220 |
| ATOM | 220 | CD | GLU | 29 | 6.740 | 4.656 | 0.671 | 1.00 | 0.27 | 1SG | 221 |
| ATOM | 221 | OE1 | GLU | 29 | 6.608 | 4.479 | −0.569 | 1.00 | 0.27 | 1SG | 222 |
| ATOM | 222 | OE2 | GLU | 29 | 7.827 | 4.508 | 1.293 | 1.00 | 0.27 | 1SG | 223 |
| ATOM | 223 | C | GLU | 29 | 2.955 | 3.923 | 2.156 | 1.00 | 0.27 | 1SG | 224 |
| ATOM | 224 | O | GLU | 29 | 2.356 | 2.887 | 1.889 | 1.00 | 0.27 | 1SG | 225 |
| ATOM | 225 | N | ILE | 30 | 3.604 | 4.094 | 3.319 | 1.00 | 0.13 | 1SG | 226 |
| ATOM | 226 | CA | ILE | 30 | 3.749 | 2.981 | 4.198 | 1.00 | 0.13 | 1SG | 227 |
| ATOM | 227 | CB | ILE | 30 | 4.608 | 3.264 | 5.400 | 1.00 | 0.13 | 1SG | 228 |
| ATOM | 228 | CG2 | ILE | 30 | 3.914 | 4.293 | 6.304 | 1.00 | 0.13 | 1SG | 229 |
| ATOM | 229 | CG1 | ILE | 30 | 4.949 | 1.947 | 6.109 | 1.00 | 0.13 | 1SG | 230 |
| ATOM | 230 | CD1 | ILE | 30 | 6.073 | 2.091 | 7.130 | 1.00 | 0.13 | 1SG | 231 |
| ATOM | 231 | C | ILE | 30 | 2.410 | 2.507 | 4.649 | 1.00 | 0.13 | 1SG | 232 |
| ATOM | 232 | O | ILE | 30 | 2.142 | 1.313 | 4.609 | 1.00 | 0.13 | 1SG | 233 |
| ATOM | 233 | N | LEU | 31 | 1.528 | 3.420 | 5.081 | 1.00 | 0.25 | 1SG | 234 |
| ATOM | 234 | CA | LEU | 31 | 0.223 | 3.065 | 5.544 | 1.00 | 0.25 | 1SG | 235 |
| ATOM | 235 | CB | LEU | 31 | −0.530 | 4.275 | 6.096 | 1.00 | 0.25 | 1SG | 236 |
| ATOM | 236 | CG | LEU | 31 | 0.094 | 4.845 | 7.377 | 1.00 | 0.25 | 1SG | 237 |
| ATOM | 237 | CD2 | LEU | 31 | 0.162 | 3.788 | 8.491 | 1.00 | 0.25 | 1SG | 238 |
| ATOM | 238 | CD1 | LEU | 31 | −0.673 | 6.088 | 7.823 | 1.00 | 0.25 | 1SG | 239 |
| ATOM | 239 | C | LEU | 31 | −0.587 | 2.557 | 4.403 | 1.00 | 0.25 | 1SG | 240 |
| ATOM | 240 | O | LEU | 31 | −1.257 | 1.534 | 4.518 | 1.00 | 0.25 | 1SG | 241 |
| ATOM | 241 | N | THR | 32 | −0.535 | 3.258 | 3.257 | 1.00. | 0.53 | 1SG | 242 |
| ATOM | 242 | CA | THR | 32 | −1.352 | 2.848 | 2.159 | 1.00 | 0.53 | 1SG | 243 |
| ATOM | 243 | CB | THR | 32 | −1.227 | 3.745 | 0.953 | 1.00 | 0.53 | 1SG | 244 |
| ATOM | 244 | OG1 | THR | 32 | −2.151 | 3.340 | −0.043 | 1.00 | 0.53 | 1SG | 245 |
| ATOM | 245 | CG2 | THR | 32 | 0.207 | 3.713 | 0.393 | 1.00 | 0.53 | 1SG | 246 |
| ATOM | 246 | C | THR | 32 | −0.961 | 1.455 | 1.790 | 1.00 | 0.53 | 1SG | 247 |
| ATOM | 247 | O | THR | 32 | −1.821 | 0.590 | 1.639 | 1.00 | 0.53 | 1SG | 248 |
| ATOM | 248 | N | VAL | 33 | 0.350 | 1.193 | 1.639 | 1.00 | 0.65 | 1SG | 249 |
| ATOM | 249 | CA | VAL | 33 | 0.769 | −0.145 | 1.355 | 1.00 | 0.65 | 1SG | 250 |
| ATOM | 250 | CB | VAL | 33 | 0.354 | −0.632 | −0.003 | 1.00 | 0.65 | 1SG | 251 |
| ATOM | 251 | CG1 | VAL | 33 | 0.991 | 0.269 | −1.074 | 1.00 | 0.65 | 1SG | 252 |
| ATOM | 252 | CG2 | VAL | 33 | 0.736 | −2.117 | −0.126 | 1.00 | 0.65 | 1SG | 253 |
| ATOM | 253 | C | VAL | 33 | 2.261 | −0.171 | 1.428 | 1.00 | 0.65 | 1SG | 254 |
| ATOM | 254 | O | VAL | 33 | 2.932 | 0.666 | 0.824 | 1.00 | 0.65 | 1SG | 255 |
| ATOM | 255 | N | GLN | 34 | 2.815 | −1.156 | 2.164 | 1.00 | 0.54 | 1SG | 256 |
| ATOM | 256 | CA | GLN | 34 | 4.238 | −1.252 | 2.346 | 1.00 | 0.54 | 1SG | 257 |
| ATOM | 257 | CB | GLN | 34 | 4.920 | 0.104 | 2.615 | 1.00 | 0.54 | 1SG | 258 |
| ATOM | 258 | CG | GLN | 34 | 6.446 | 0.039 | 2.681 | 1.00 | 0.54 | 1SG | 259 |
| ATOM | 259 | CD | GLN | 34 | 6.970 | 0.023 | 1.253 | 1.00 | 0.54 | 1SG | 260 |
| ATOM | 260 | OE1 | GLN | 34 | 6.201 | 0.038 | 0.293 | 1.00 | 0.54 | 1SG | 261 |
| ATOM | 261 | NE2 | GLN | 34 | 8.322 | −0.005 | 1.106 | 1.00 | 0.54 | 1SG | 262 |
| ATOM | 262 | C | GLN | 34 | 4.406 | −2.038 | 3.598 | 1.00 | 0.54 | 1SG | 263 |
| ATOM | 263 | O | GLN | 34 | 4.079 | −3.221 | 3.674 | 1.00 | 0.54 | 1SG | 264 |
| ATOM | 264 | N | PHE | 35 | 4.928 | −1.349 | 4.632 | 1.00 | 0.51 | 1SG | 265 |
| ATOM | 265 | CA | PHE | 35 | 5.028 | −1.878 | 5.958 | 1.00 | 0.51 | 1SG | 266 |
| ATOM | 266 | CB | PHE | 35 | 5.692 | −0.915 | 6.956 | 1.00 | 0.51 | 1SG | 267 |
| ATOM | 267 | CG | PHE | 35 | 7.154 | −0.916 | 6.661 | 1.00 | 0.51 | 1SG | 268 |
| ATOM | 268 | CD1 | PHE | 35 | 7.972 | −1.862 | 7.234 | 1.00 | 0.51 | 1SG | 269 |
| ATOM | 269 | CD2 | PHE | 35 | 7.710 | 0.016 | 5.815 | 1.00 | 0.51 | 1SG | 270 |
| ATOM | 270 | CE1 | PHE | 35 | 9.322 | −1.878 | 6.973 | 1.00 | 0.51 | 1SG | 271 |
| ATOM | 271 | CE2 | PHE | 35 | 9.060 | 0.006 | 5.548 | 1.00 | 0.51 | 1SG | 272 |
| ATOM | 272 | CZ | PHE | 35 | 9.868 | −0.942 | 6.128 | 1.00 | 0.51 | 1SG | 273 |
| ATOM | 273 | C | PHE | 35 | 3.617 | −2.108 | 6.361 | 1.00 | 0.51 | 1SG | 274 |
| ATOM | 274 | O | PHE | 35 | 3.351 | −2.909 | 7.255 | 1.00 | 0.51 | 1SG | 275 |
| ATOM | 275 | N | LYS | 36 | 2.718 | −1.332 | 5.709 | 1.00 | 0.64 | 1SG | 276 |
| ATOM | 276 | CA | LYS | 36 | 1.290 | −1.422 | 5.737 | 1.00 | 0.64 | 1SG | 277 |
| ATOM | 277 | CB | LYS | 36 | 0.703 | −0.994 | 4.372 | 1.00 | 0.64 | 1SG | 278 |
| ATOM | 278 | CG | LYS | 36 | −0.802 | −1.146 | 4.140 | 1.00 | 0.64 | 1SG | 279 |
| ATOM | 279 | CD | LYS | 36 | −1.280 | −2.580 | 3.894 | 1.00 | 0.64 | 1SG | 280 |
| ATOM | 280 | CE | LYS | 36 | −0.890 | −3.129 | 2.520 | 1.00 | 0.64 | 1SG | 281 |
| ATOM | 281 | NZ | LYS | 36 | −1.381 | −4.517 | 2.370 | 1.00 | 0.64 | 1SG | 282 |
| ATOM | 282 | C | LYS | 36 | 0.952 | −2.848 | 5.970 | 1.00 | 0.64 | 1SG | 283 |
| ATOM | 283 | O | LYS | 36 | 0.931 | −3.673 | 5.060 | 1.00 | 0.64 | 1SG | 284 |
| ATOM | 284 | N | LYS | 37 | 0.757 | −3.165 | 7.255 | 1.00 | 0.61 | 1SG | 285 |
| ATOM | 285 | CA | LYS | 37 | 0.327 | −4.452 | 7.677 | 1.00 | 0.61 | 1SG | 286 |
| ATOM | 286 | CB | LYS | 37 | 0.355 | −4.584 | 9.210 | 1.00 | 0.61 | 1SG | 287 |
| ATOM | 287 | CG | LYS | 37 | −0.331 | −5.830 | 9.774 | 1.00 | 0.61 | 1SG | 288 |
| ATOM | 288 | CD | LYS | 37 | −1.861 | −5.790 | 9.740 | 1.00 | 0.61 | 1SG | 289 |
| ATOM | 289 | CE | LYS | 37 | −2.520 | −7.061 | 10.275 | 1.00 | 0.61 | 1SG | 290 |
| ATOM | 290 | NZ | LYS | 37 | −3.957 | −6.823 | 10.534 | 1.00 | 0.61 | 1SG | 291 |
| ATOM | 291 | C | LYS | 37 | −1.068 | −4.493 | 7.222 | 1.00 | 0.61 | 1SG | 292 |
| ATOM | 292 | O | LYS | 37 | −1.599 | −5.568 | 6.940 | 1.00 | 0.61 | 1SG | 293 |
| ATOM | 293 | N | ARG | 38 | −1.627 | −3.260 | 7.152 | 1.00 | 0.44 | 1SG | 294 |
| ATOM | 294 | CA | ARG | 38 | −2.966 | −2.877 | 6.823 | 1.00 | 0.44 | 1SG | 295 |
| ATOM | 295 | CB | ARG | 38 | −3.008 | −1.557 | 6.043 | 1.00 | 0.44 | 1SG | 296 |
| ATOM | 296 | CG | ARG | 38 | −4.389 | −1.079 | 5.621 | 1.00 | 0.44 | 1SG | 297 |
| ATOM | 297 | CD | ARG | 38 | −4.352 | 0.305 | 4.976 | 1.00 | 0.44 | 1SG | 298 |
| ATOM | 298 | NE | ARG | 38 | −3.870 | 1.263 | 6.010 | 1.00 | 0.44 | 1SG | 299 |
| ATOM | 299 | CZ | ARG | 38 | −3.673 | 2.576 | 5.692 | 1.00 | 0.44 | 1SG | 300 |
| ATOM | 300 | NH1 | ARG | 38 | −3.919 | 3.012 | 4.422 | 1.00 | 0.44 | 1SG | 301 |
| ATOM | 301 | NH2 | ARG | 38 | −3.242 | 3.452 | 6.645 | 1.00 | 0.44 | 1SG | 302 |
| ATOM | 302 | C | ARG | 38 | −3.567 | −3.968 | 6.037 | 1.00 | 0.44 | 1SG | 303 |
| ATOM | 303 | O | ARG | 38 | −3.204 | −4.265 | 4.898 | 1.00 | 0.44 | 1SG | 304 |
| ATOM | 304 | N | PRO | 39 | −4.481 | −4.559 | 6.751 | 1.00 | 0.50 | 1SG | 305 |
| ATOM | 305 | CA | PRO | 39 | −5.119 | −5.774 | 6.350 | 1.00 | 0.50 | 1SG | 306 |
| ATOM | 306 | CD | PRO | 39 | −5.313 | −3.783 | 7.656 | 1.00 | 0.50 | 1SG | 307 |
| ATOM | 307 | CB | PRO | 39 | −6.299 | −5.961 | 7.302 | 1.00 | 0.50 | 1SG | 308 |
| ATOM | 308 | CG | PRO | 39 | −6.661 | −4.519 | 7.697 | 1.00 | 0.50 | 1SG | 309 |
| ATOM | 309 | C | PRO | 39 | −5.558 | −5.635 | 4.946 | 1.00 | 0.50 | 1SG | 310 |
| ATOM | 310 | O | PRO | 39 | −5.515 | −6.624 | 4.218 | 1.00 | 0.50 | 1SG | 311 |
| ATOM | 311 | N | ARG | 40 | −5.984 | −4.429 | 4.538 | 1.00 | 0.60 | 1SG | 312 |
| ATOM | 312 | CA | ARG | 40 | −6.363 | −4.353 | 3.170 | 1.00 | 0.60 | 1SG | 313 |
| ATOM | 313 | CB | ARG | 40 | −7.479 | −5.345 | 2.794 | 1.00 | 0.60 | 1SG | 314 |
| ATOM | 314 | CG | ARG | 40 | −8.777 | −5.105 | 3.569 | 1.00 | 0.60 | 1SG | 315 |
| ATOM | 315 | CD | ARG | 40 | −9.863 | −6.146 | 3.301 | 1.00 | 0.60 | 1SG | 316 |
| ATOM | 316 | NE | ARG | 40 | −9.433 | −7.392 | 3.988 | 1.00 | 0.60 | 1SG | 317 |
| ATOM | 317 | CZ | ARG | 40 | −9.691 | −7.565 | 5.317 | 1.00 | 0.60 | 1SG | 318 |
| ATOM | 318 | NH1 | ARG | 40 | −10.410 | −6.631 | 6.003 | 1.00 | 0.60 | 1SG | 319 |
| ATOM | 319 | NH2 | ARG | 40 | −9.208 | −8.665 | 5.963 | 1.00 | 0.60 | 1SG | 320 |
| ATOM | 320 | C | ARG | 40 | −6.904 | −3.002 | 2.884 | 1.00 | 0.60 | 1SG | 321 |
| ATOM | 321 | O | ARG | 40 | −6.544 | −1.995 | 3.493 | 1.00 | 0.60 | 1SG | 322 |
| ATOM | 322 | N | TRP | 41 | −7.826 | −3.020 | 1.914 | 1.00 | 0.43 | 1SG | 323 |
| ATOM | 323 | CA | TRP | 41 | −8.566 | −1.945 | 1.349 | 1.00 | 0.43 | 1SG | 324 |
| ATOM | 324 | CB | TRP | 41 | −9.575 | −2.528 | 0.342 | 1.00 | 0.43 | 1SG | 325 |
| ATOM | 325 | CG | TRP | 41 | −10.581 | −1.590 | −0.269 | 1.00 | 0.43 | 1SG | 326 |
| ATOM | 326 | CD2 | TRP | 41 | −11.307 | −1.896 | −1.468 | 1.00 | 0.43 | 1SG | 327 |
| ATOM | 327 | CD1 | TRP | 41 | −11.005 | −0.362 | 0.138 | 1.00 | 0.43 | 1SG | 328 |
| ATOM | 328 | NE1 | TRP | 41 | −11.947 | 0.124 | −0.739 | 1.00 | 0.43 | 1SG | 329 |
| ATOM | 329 | CE2 | TRP | 41 | −12.144 | −0.813 | −1.732 | 1.00 | 0.43 | 1SG | 330 |
| ATOM | 330 | CE3 | TRP | 41 | −11.277 | −2.993 | −2.281 | 1.00 | 0.43 | 1SG | 331 |
| ATOM | 331 | CZ2 | TRP | 41 | −12.969 | −0.810 | −2.821 | 1.00 | 0.43 | 1SG | 332 |
| ATOM | 332 | CZ3 | TRP | 41 | −12.110 | −2.987 | −3.378 | 1.00 | 0.43 | 1SG | 333 |
| ATOM | 333 | CH2 | TRP | 41 | −12.939 | −1.916 | −3.642 | 1.00 | 0.43 | 1SG | 334 |
| ATOM | 334 | C | TRP | 41 | −9.314 | −1.301 | 2.460 | 1.00 | 0.43 | 1SG | 335 |
| ATOM | 335 | O | TRP | 41 | −9.489 | −0.083 | 2.474 | 1.00 | 0.43 | 1SG | 336 |
| ATOM | 336 | N | THR | 42 | −9.758 | −2.109 | 3.439 | 1.00 | 0.27 | 1SG | 337 |
| ATOM | 337 | CA | THR | 42 | −10.552 | −1.551 | 4.484 | 1.00 | 0.27 | 1SG | 338 |
| ATOM | 338 | CB | THR | 42 | −11.123 | −2.589 | 5.428 | 1.00 | 0.27 | 1SG | 339 |
| ATOM | 339 | OG1 | THR | 42 | −10.132 | −3.487 | 5.903 | 1.00 | 0.27 | 1SG | 340 |
| ATOM | 340 | CG2 | THR | 42 | −12.204 | −3.378 | 4.670 | 1.00 | 0.27 | 1SG | 341 |
| ATOM | 341 | C | THR | 42 | −9.817 | −0.429 | 5.172 | 1.00 | 0.27 | 1SG | 342 |
| ATOM | 342 | O | THR | 42 | −10.444 | 0.612 | 5.328 | 1.00 | 0.27 | 1SG | 343 |
| ATOM | 343 | N | PRO | 43 | −8.598 | −0.471 | 5.648 | 1.00 | 0.27 | 1SG | 344 |
| ATOM | 344 | CA | PRO | 43 | −8.062 | 0.793 | 6.084 | 1.00 | 0.27 | 1SG | 345 |
| ATOM | 345 | CD | PRO | 43 | −8.152 | −1.550 | 6.511 | 1.00 | 0.27 | 1SG | 346 |
| ATOM | 346 | CB | PRO | 43 | −7.194 | 0.508 | 7.312 | 1.00 | 0.27 | 1SG | 347 |
| ATOM | 347 | CG | PRO | 43 | −6.930 | −1.001 | 7.254 | 1.00 | 0.27 | 1SG | 348 |
| ATOM | 348 | C | PRO | 43 | −7.328 | 1.499 | 4.978 | 1.00 | 0.27 | 1SG | 349 |
| ATOM | 349 | O | PRO | 43 | −7.083 | 2.698 | 5.092 | 1.00 | 0.27 | 1SG | 350 |
| ATOM | 350 | N | HIS | 44 | −6.942 | 0.762 | 3.918 | 1.00 | 0.25 | 1SG | 351 |
| ATOM | 351 | CA | HIS | 44 | −6.132 | 1.260 | 2.835 | 1.00 | 0.25 | 1SG | 352 |
| ATOM | 352 | ND1 | HIS | 44 | −4.061 | −0.451 | 0.149 | 1.00 | 0.25 | 1SG | 353 |
| ATOM | 353 | CG | HIS | 44 | −5.055 | 0.381 | 0.620 | 1.00 | 0.25 | 1SG | 354 |
| ATOM | 354 | CB | HIS | 44 | −5.851 | 0.097 | 1.860 | 1.00 | 0.25 | 1SG | 355 |
| ATOM | 355 | NE2 | HIS | 44 | −4.268 | 1.190 | −1.333 | 1.00 | 0.25 | 1SG | 356 |
| ATOM | 356 | CD2 | HIS | 44 | −5.165 | 1.380 | −0.295 | 1.00 | 0.25 | 1SG | 357 |
| ATOM | 357 | CE1 | HIS | 44 | −3.628 | 0.079 | −1.021 | 1.00 | 0.25 | 1SG | 358 |
| ATOM | 358 | C | HIS | 44 | −6.835 | 2.333 | 2.064 | 1.00 | 0.25 | 1SG | 359 |
| ATOM | 359 | O | HIS | 44 | −6.439 | 3.497 | 2.084 | 1.00 | 0.25 | 1SG | 360 |
| ATOM | 360 | N | ASP | 45 | −7.942 | 1.962 | 1.398 | 1.00 | 0.33 | 1SG | 361 |
| ATOM | 361 | CA | ASP | 45 | −8.658 | 2.893 | 0.572 | 1.00 | 0.33 | 1SG | 362 |
| ATOM | 362 | CB | ASP | 45 | −9.729 | 2.248 | −0.317 | 1.00 | 0.33 | 1SG | 363 |
| ATOM | 363 | CG | ASP | 45 | −9.018 | 1.423 | −1.380 | 1.00 | 0.33 | 1SG | 364 |
| ATOM | 364 | OD1 | ASP | 45 | −7.758 | 1.425 | −1.388 | 1.00 | 0.33 | 1SG | 365 |
| ATOM | 365 | OD2 | ASP | 45 | −9.727 | 0.785 | −2.203 | 1.00 | 0.33 | 1SG | 366 |
| ATOM | 366 | C | ASP | 45 | −9.334 | 3.865 | 1.466 | 1.00 | 0.33 | 1SG | 367 |
| ATOM | 367 | O | ASP | 45 | −9.554 | 5.023 | 1.121 | 1.00 | 0.33 | 1SG | 368 |
| ATOM | 368 | N | TYR | 46 | −9.658 | 3.390 | 2.670 | 1.00 | 0.43 | 1SG | 369 |
| ATOM | 369 | CA | TYR | 46 | −10.351 | 4.114 | 3.683 | 1.00 | 0.43 | 1SG | 370 |
| ATOM | 370 | CB | TYR | 46 | −10.936 | 3.248 | 4.804 | 1.00 | 0.43 | 1SG | 371 |
| ATOM | 371 | CG | TYR | 46 | −12.193 | 2.666 | 4.239 | 1.00 | 0.43 | 1SG | 372 |
| ATOM | 372 | CD1 | TYR | 46 | −12.185 | 1.541 | 3.443 | 1.00 | 0.43 | 1SG | 373 |
| ATOM | 373 | CD2 | TYR | 46 | −13.398 | 3.283 | 4.482 | 1.00 | 0.43 | 1SG | 374 |
| ATOM | 374 | CE1 | TYR | 46 | −13.352 | 1.026 | 2.929 | 1.00 | 0.43 | 1SG | 375 |
| ATOM | 375 | CE2 | TYR | 46 | −14.570 | 2.776 | 3.972 | 1.00 | 0.43 | 1SG | 376 |
| ATOM | 376 | CZ | TYR | 46 | −14.549 | 1.643 | 3.197 | 1.00 | 0.43 | 1SG | 377 |
| ATOM | 377 | OH | TYR | 46 | −15.749 | 1.118 | 2.672 | 1.00 | 0.43 | 1SG | 378 |
| ATOM | 378 | C | TYR | 46 | −9.507 | 5.219 | 4.216 | 1.00 | 0.43 | 1SG | 379 |
| ATOM | 379 | O | TYR | 46 | −9.760 | 5.687 | 5.322 | 1.00 | 0.43 | 1SG | 380 |
| ATOM | 380 | N | ILE | 47 | −8.376 | 5.524 | 3.542 | 1.00 | 0.29 | 1SG | 381 |
| ATOM | 381 | CA | ILE | 47 | −7.563 | 6.632 | 3.957 | 1.00 | 0.29 | 1SG | 382 |
| ATOM | 382 | CB | ILE | 47 | −6.109 | 6.464 | 3.608 | 1.00 | 0.29 | 1SG | 383 |
| ATOM | 383 | CG2 | ILE | 47 | −5.968 | 6.366 | 2.079 | 1.00 | 0.29 | 1SG | 384 |
| ATOM | 384 | CG1 | ILE | 47 | −5.270 | 7.578 | 4.252 | 1.00 | 0.29 | 1SG | 385 |
| ATOM | 385 | CD1 | ILE | 47 | −3.767 | 7.320 | 4.179 | 1.00 | 0.29 | 1SG | 386 |
| ATOM | 386 | C | ILE | 47 | −8.074 | 7.894 | 3.312 | 1.00 | 0.29 | 1SG | 387 |
| ATOM | 387 | O | ILE | 47 | −8.163 | 8.007 | 2.089 | 1.00 | 0.29 | 1SG | 388 |
| ATOM | 388 | N | ASN | 48 | −8.445 | 8.889 | 4.145 | 1.00 | 0.22 | 1SG | 389 |
| ATOM | 389 | CA | ASN | 48 | −8.937 | 10.133 | 3.629 | 1.00 | 0.22 | 1SG | 390 |
| ATOM | 390 | CB | ASN | 48 | −10.188 | 10.666 | 4.351 | 1.00 | 0.22 | 1SG | 391 |
| ATOM | 391 | CG | ASN | 48 | −10.626 | 11.938 | 3.633 | 1.00 | 0.22 | 1SG | 392 |
| ATOM | 392 | OD1 | ASN | 48 | −10.066 | 12.305 | 2.600 | 1.00 | 0.22 | 1SG | 393 |
| ATOM | 393 | ND2 | ASN | 48 | −11.648 | 12.636 | 4.196 | 1.00 | 0.22 | 1SG | 394 |
| ATOM | 394 | C | ASN | 48 | −7.866 | 11.143 | 3.849 | 1.00 | 0.22 | 1SG | 395 |
| ATOM | 395 | O | ASN | 48 | −7.361 | 11.303 | 4.959 | 1.00 | 0.22 | 1SG | 396 |
| ATOM | 396 | N | MET | 49 | −7.485 | 11.860 | 2.779 | 1.00 | 0.41 | 1SG | 397 |
| ATOM | 397 | CA | MET | 49 | −6.456 | 12.835 | 2.936 | 1.00 | 0.41 | 1SG | 398 |
| ATOM | 398 | CB | MET | 49 | −5.414 | 12.776 | 1.819 | 1.00 | 0.41 | 1SG | 399 |
| ATOM | 399 | CG | MET | 49 | −6.043 | 12.977 | 0.442 | 1.00 | 0.41 | 1SG | 400 |
| ATOM | 400 | SD | MET | 49 | −4.869 | 12.918 | −0.932 | 1.00 | 0.41 | 1SG | 401 |
| ATOM | 401 | CE | MET | 49 | −4.060 | 14.479 | −0.491 | 1.00 | 0.41 | 1SG | 402 |
| ATOM | 402 | C | MET | 49 | −7.091 | 14.177 | 2.845 | 1.00 | 0.41 | 1SG | 403 |
| ATOM | 403 | O | MET | 49 | −7.818 | 14.474 | 1.898 | 1.00 | 0.41 | 1SG | 404 |
| ATOM | 404 | N | THR | 50 | −6.848 | 15.025 | 3.856 | 1.00 | 0.66 | 1SG | 405 |
| ATOM | 405 | CA | THR | 50 | −7.361 | 16.349 | 3.741 | 1.00 | 0.66 | 1SG | 406 |
| ATOM | 406 | CB | THR | 50 | −8.058 | 16.876 | 4.974 | 1.00 | 0.66 | 1SG | 407 |
| ATOM | 407 | OG1 | THR | 50 | −8.710 | 18.101 | 4.672 | 1.00 | 0.66 | 1SG | 408 |
| ATOM | 408 | CG2 | THR | 50 | −7.045 | 17.088 | 6.113 | 1.00 | 0.66 | 1SG | 409 |
| ATOM | 409 | C | THR | 50 | −6.177 | 17.202 | 3.452 | 1.00 | 0.66 | 1SG | 410 |
| ATOM | 410 | O | THR | 50 | −5.158 | 17.129 | 4.137 | 1.00 | 0.66 | 1SG | 411 |
| ATOM | 411 | N | ARG | 51 | −6.234 | 17.992 | 2.371 | 1.00 | 0.70 | 1SG | 412 |
| ATOM | 412 | CA | ARG | 51 | −5.102 | 18.840 | 2.183 | 1.00 | 0.70 | 1SG | 413 |
| ATOM | 413 | CB | ARG | 51 | −3.986 | 18.257 | 1.290 | 1.00 | 0.70 | 1SG | 414 |
| ATOM | 414 | CG | ARG | 51 | −4.401 | 17.889 | −0.131 | 1.00 | 0.70 | 1SG | 415 |
| ATOM | 415 | CD | ARG | 51 | −3.269 | 17.272 | −0.958 | 1.00 | 0.70 | 1SG | 416 |
| ATOM | 416 | NE | ARG | 51 | −2.203 | 18.295 | −1.130 | 1.00 | 0.70 | 1SG | 417 |
| ATOM | 417 | CZ | ARG | 51 | −0.967 | 17.929 | −1.580 | 1.00 | 0.70 | 1SG | 418 |
| ATOM | 418 | NH1 | ARG | 51 | −0.682 | 16.613 | −1.802 | 1.00 | 0.70 | 1SG | 419 |
| ATOM | 419 | NH2 | ARG | 51 | −0.016 | 18.880 | −1.812 | 1.00 | 0.70 | 1SG | 420 |
| ATOM | 420 | C | ARG | 51 | −5.563 | 20.148 | 1.642 | 1.00 | 0.70 | 1SG | 421 |
| ATOM | 421 | O | ARG | 51 | −6.519 | 20.223 | 0.871 | 1.00 | 0.70 | 1SG | 422 |
| ATOM | 422 | N | ASP | 52 | −4.934 | 21.239 | 2.116 | 1.00 | 0.47 | 1SG | 423 |
| ATOM | 423 | CA | ASP | 52 | −5.268 | 22.515 | 1.562 | 1.00 | 0.47 | 1SG | 424 |
| ATOM | 424 | CB | ASP | 52 | −4.596 | 23.697 | 2.275 | 1.00 | 0.47 | 1SG | 425 |
| ATOM | 425 | CG | ASP | 52 | −5.019 | 25.006 | 1.614 | 1.00 | 0.47 | 1SG | 426 |
| ATOM | 426 | OD1 | ASP | 52 | −5.832 | 24.964 | 0.652 | 1.00 | 0.47 | 1SG | 427 |
| ATOM | 427 | OD2 | ASP | 52 | −4.529 | 26.073 | 2.072 | 1.00 | 0.47 | 1SG | 428 |
| ATOM | 428 | C | ASP | 52 | −4.740 | 22.497 | 0.174 | 1.00 | 0.47 | 1SG | 429 |
| ATOM | 429 | O | ASP | 52 | −5.425 | 22.859 | −0.780 | 1.00 | 0.47 | 1SG | 430 |
| ATOM | 430 | N | CYS | 53 | −3.488 | 22.027 | 0.049 | 1.00 | 0.39 | 1SG | 431 |
| ATOM | 431 | CA | CYS | 53 | −2.795 | 21.935 | −1.192 | 1.00 | 0.39 | 1SG | 432 |
| ATOM | 432 | CB | CYS | 53 | −1.301 | 21.644 | −0.981 | 1.00 | 0.39 | 1SG | 433 |
| ATOM | 433 | SG | CYS | 53 | −0.469 | 22.937 | −0.005 | 1.00 | 0.39 | 1SG | 434 |
| ATOM | 434 | C | CYS | 53 | −3.420 | 20.771 | −1.883 | 1.00 | 0.39 | 1SG | 435 |
| ATOM | 435 | O | CYS | 53 | −4.460 | 20.293 | −1.438 | 1.00 | 0.39 | 1SG | 436 |
| ATOM | 436 | N | ALA | 54 | −2.857 | 20.296 | −3.014 | 1.00 | 0.45 | 1SG | 437 |
| ATOM | 437 | CA | ALA | 54 | −3.514 | 19.155 | −3.584 | 1.00 | 0.45 | 1SG | 438 |
| ATOM | 438 | CB | ALA | 54 | −4.714 | 19.523 | −4.473 | 1.00 | 0.45 | 1SG | 439 |
| ATOM | 439 | C | ALA | 54 | −2.571 | 18.363 | −4.437 | 1.00 | 0.45 | 1SG | 440 |
| ATOM | 440 | O | ALA | 54 | −1.744 | 18.919 | −5.158 | 1.00 | 0.45 | 1SG | 441 |
| ATOM | 441 | N | SER | 55 | −2.662 | 17.018 | −4.330 | 1.00 | 0.47 | 1SG | 442 |
| ATOM | 442 | CA | SER | 55 | −1.935 | 16.114 | −5.181 | 1.00 | 0.47 | 1SG | 443 |
| ATOM | 443 | CB | SER | 55 | −0.461 | 16.502 | −5.386 | 1.00 | 0.47 | 1SG | 444 |
| ATOM | 444 | OG | SER | 55 | 0.180 | 15.560 | −6.235 | 1.00 | 0.47 | 1SG | 445 |
| ATOM | 445 | C | SER | 55 | −1.951 | 14.767 | −4.524 | 1.00 | 0.47 | 1SG | 446 |
| ATOM | 446 | O | SER | 55 | −1.622 | 14.654 | −3.345 | 1.00 | 0.47 | 1SG | 447 |
| ATOM | 447 | N | PHE | 56 | −2.339 | 13.697 | −5.255 | 1.00 | 0.54 | 1SG | 448 |
| ATOM | 448 | CA | PHE | 56 | −2.266 | 12.407 | −4.628 | 1.00 | 0.54 | 1SG | 449 |
| ATOM | 449 | CB | PHE | 56 | −3.062 | 12.315 | −3.326 | 1.00 | 0.54 | 1SG | 450 |
| ATOM | 450 | CG | PHE | 56 | −2.637 | 11.059 | −2.655 | 1.00 | 0.54 | 1SG | 451 |
| ATOM | 451 | CD1 | PHE | 56 | −1.430 | 11.007 | −2.000 | 1.00 | 0.54 | 1SG | 452 |
| ATOM | 452 | CD2 | PHE | 56 | −3.440 | 9.946 | −2.666 | 1.00 | 0.54 | 1SG | 453 |
| ATOM | 453 | CE1 | PHE | 56 | −1.020 | 9.854 | −1.376 | 1.00 | 0.54 | 1SG | 454 |
| ATOM | 454 | CE2 | PHE | 56 | −3.032 | 8.794 | −2.043 | 1.00 | 0.54 | 1SG | 455 |
| ATOM | 455 | CZ | PHE | 56 | −1.822 | 8.740 | −1.397 | 1.00 | 0.54 | 1SG | 456 |
| ATOM | 456 | C | PHE | 56 | −2.797 | 11.373 | −5.572 | 1.00 | 0.54 | 1SG | 457 |
| ATOM | 457 | O | PHE | 56 | −3.527 | 11.682 | −6.511 | 1.00 | 0.54 | 1SG | 458 |
| ATOM | 458 | N | ILE | 57 | −2.453 | 10.096 | −5.308 | 1.00 | 0.53 | 1SG | 459 |
| ATOM | 459 | CA | ILE | 57 | −2.817 | 8.999 | −6.157 | 1.00 | 0.53 | 1SG | 460 |
| ATOM | 460 | CB | ILE | 57 | −1.844 | 7.851 | −5.985 | 1.00 | 0.53 | 1SG | 461 |
| ATOM | 461 | CG2 | ILE | 57 | −2.305 | 7.058 | −4.757 | 1.00 | 0.53 | 1SG | 462 |
| ATOM | 462 | CG1 | ILE | 57 | −1.627 | 6.981 | −7.249 | 1.00 | 0.53 | 1SG | 463 |
| ATOM | 463 | CD1 | ILE | 57 | −2.737 | 6.028 | −7.687 | 1.00 | 0.53 | 1SG | 464 |
| ATOM | 464 | C | ILE | 57 | −4.215 | 8.590 | −5.784 | 1.00 | 0.53 | 1SG | 465 |
| ATOM | 465 | O | ILE | 57 | −4.838 | 9.175 | −4.898 | 1.00 | 0.53 | 1SG | 466 |
| ATOM | 466 | N | ARG | 58 | −4.746 | 7.580 | −6.492 | 1.00 | 0.42 | 1SG | 467 |
| ATOM | 467 | CA | ARG | 58 | −6.075 | 7.059 | −6.356 | 1.00 | 0.42 | 1SG | 468 |
| ATOM | 468 | CB | ARG | 58 | −6.418 | 5.992 | −7.400 | 1.00 | 0.42 | 1SG | 469 |
| ATOM | 469 | CG | ARG | 58 | −7.842 | 5.462 | −7.232 | 1.00 | 0.42 | 1SG | 470 |
| ATOM | 470 | CD | ARG | 58 | −8.100 | 4.154 | −7.974 | 1.00 | 0.42 | 1SG | 471 |
| ATOM | 471 | NE | ARG | 58 | −9.527 | 3.783 | −7.754 | 1.00 | 0.42 | 1SG | 472 |
| ATOM | 472 | CZ | ARG | 58 | −9.889 | 3.047 | −6.663 | 1.00 | 0.42 | 1SG | 473 |
| ATOM | 473 | NH1 | ARG | 58 | −8.948 | 2.675 | −5.747 | 1.00 | 0.42 | 1SG | 474 |
| ATOM | 474 | NH2 | ARG | 58 | −11.193 | 2.681 | −6.490 | 1.00 | 0.42 | 1SG | 475 |
| ATOM | 475 | C | ARG | 58 | −6.247 | 6.400 | −5.029 | 1.00 | 0.42 | 1SG | 476 |
| ATOM | 476 | O | ARG | 58 | −7.362 | 6.335 | −4.514 | 1.00 | 0.42 | 1SG | 477 |
| ATOM | 477 | N | THR | 59 | −5.150 | 5.888 | −4.439 | 1.00 | 0.45 | 1SG | 478 |
| ATOM | 478 | CA | THR | 59 | −5.271 | 5.127 | −3.229 | 1.00 | 0.45 | 1SG | 479 |
| ATOM | 479 | CB | THR | 59 | −3.974 | 4.660 | −2.644 | 1.00 | 0.45 | 1SG | 480 |
| ATOM | 480 | OG1 | THR | 59 | −4.250 | 3.836 | −1.524 | 1.00 | 0.45 | 1SG | 481 |
| ATOM | 481 | CG2 | THR | 59 | −3.137 | 5.862 | −2.197 | 1.00 | 0.45 | 1SG | 482 |
| ATOM | 482 | C | THR | 59 | −5.976 | 5.935 | −2.191 | 1.00 | 0.45 | 1SG | 483 |
| ATOM | 483 | O | THR | 59 | −6.696 | 5.378 | −1.363 | 1.00 | 0.45 | 1SG | 484 |
| ATOM | 484 | N | ARG | 60 | −5.797 | 7.267 | −2.188 | 1.00 | 0.45 | 1SG | 485 |
| ATOM | 485 | CA | ARG | 60 | −6.484 | 8.028 | −1.185 | 1.00 | 0.45 | 1SG | 486 |
| ATOM | 486 | CB | ARG | 60 | −5.711 | 9.224 | −0.630 | 1.00 | 0.45 | 1SG | 487 |
| ATOM | 487 | CG | ARG | 60 | −4.651 | 8.926 | 0.418 | 1.00 | 0.45 | 1SG | 488 |
| ATOM | 488 | CD | ARG | 60 | −4.159 | 10.241 | 1.007 | 1.00 | 0.45 | 1SG | 489 |
| ATOM | 489 | NE | ARG | 60 | −3.114 | 9.964 | 2.019 | 1.00 | 0.45 | 1SG | 490 |
| ATOM | 490 | CZ | ARG | 60 | −1.991 | 10.731 | 1.971 | 1.00 | 0.45 | 1SG | 491 |
| ATOM | 491 | NH1 | ARG | 60 | −1.882 | 11.673 | 0.989 | 1.00 | 0.45 | 1SG | 492 |
| ATOM | 492 | NH2 | ARG | 60 | −0.997 | 10.558 | 2.889 | 1.00 | 0.45 | 1SG | 493 |
| ATOM | 493 | C | ARG | 60 | −7.707 | 8.660 | −1.757 | 1.00 | 0.45 | 1SG | 494 |
| ATOM | 494 | O | ARG | 60 | −7.899 | 8.727 | −2.970 | 1.00 | 0.45 | 1SG | 495 |
| ATOM | 495 | N | LYS | 61 | −8.580 | 9.126 | −0.839 | 1.00 | 0.33 | 1SG | 496 |
| ATOM | 496 | CA | LYS | 61 | −9.756 | 9.873 | −1.172 | 1.00 | 0.33 | 1SG | 497 |
| ATOM | 497 | CB | LYS | 61 | −11.011 | 9.454 | −0.386 | 1.00 | 0.33 | 1SG | 498 |
| ATOM | 498 | CG | LYS | 61 | −12.263 | 10.243 | −0.775 | 1.00 | 0.33 | 1SG | 499 |
| ATOM | 499 | CD | LYS | 61 | −13.569 | 9.598 | −0.304 | 1.00 | 0.33 | 1SG | 500 |
| ATOM | 500 | CE | LYS | 61 | −13.799 | 9.697 | 1.204 | 1.00 | 0.33 | 1SG | 501 |
| ATOM | 501 | NZ | LYS | 61 | −15.080 | 9.047 | 1.560 | 1.00 | 0.33 | 1SG | 502 |
| ATOM | 502 | C | LYS | 61 | −9.412 | 11.273 | −0.788 | 1.00 | 0.33 | 1SG | 503 |
| ATOM | 503 | O | LYS | 61 | −8.806 | 11.505 | 0.257 | 1.00 | 0.33 | 1SG | 504 |
| ATOM | 504 | N | TYR | 62 | −9.789 | 12.254 | −1.626 | 1.00 | 0.21 | 1SG | 505 |
| ATOM | 505 | CA | TYR | 62 | −9.333 | 13.586 | −1.365 | 1.00 | 0.21 | 1SG | 506 |
| ATOM | 506 | CB | TYR | 62 | −8.678 | 14.212 | −2.607 | 1.00 | 0.21 | 1SG | 507 |
| ATOM | 507 | CG | TYR | 62 | −8.374 | 15.636 | −2.319 | 1.00 | 0.21 | 1SG | 508 |
| ATOM | 508 | CD1 | TYR | 62 | −7.349 | 15.981 | −1.470 | 1.00 | 0.21 | 1SG | 509 |
| ATOM | 509 | CD2 | TYR | 62 | −9.109 | 16.628 | −2.922 | 1.00 | 0.21 | 1SG | 510 |
| ATOM | 510 | CE1 | TYR | 62 | −7.074 | 17.303 | −1.216 | 1.00 | 0.21 | 1SG | 511 |
| ATOM | 511 | CE2 | TYR | 62 | −8.834 | 17.948 | −2.669 | 1.00 | 0.21 | 1SG | 512 |
| ATOM | 512 | CZ | TYR | 62 | −7.818 | 18.289 | −1.813 | 1.00 | 0.21 | 1SG | 513 |
| ATOM | 513 | OH | TYR | 62 | −7.537 | 19.647 | −1.552 | 1.00 | 0.21 | 1SG | 514 |
| ATOM | 514 | C | TYR | 62 | −10.454 | 14.471 | −0.920 | 1.00 | 0.21 | 1SG | 515 |
| ATOM | 515 | O | TYR | 62 | −11.584 | 14.364 | −1.395 | 1.00 | 0.21 | 1SG | 516 |
| ATOM | 516 | N | ILE | 63 | −10.142 | 15.353 | 0.053 | 1.00 | 0.16 | 1SG | 517 |
| ATOM | 517 | CA | ILE | 63 | −11.046 | 16.352 | 0.539 | 1.00 | 0.16 | 1SG | 518 |
| ATOM | 518 | CB | ILE | 63 | −11.435 | 16.219 | 1.980 | 1.00 | 0.16 | 1SG | 519 |
| ATOM | 519 | CG2 | ILE | 63 | −10.161 | 16.194 | 2.838 | 1.00 | 0.16 | 1SG | 520 |
| ATOM | 520 | CG1 | ILE | 63 | −12.405 | 17.361 | 2.327 | 1.00 | 0.16 | 1SG | 521 |
| ATOM | 521 | CD1 | ILE | 63 | −12.897 | 17.348 | 3.769 | 1.00 | 0.16 | 1SG | 522 |
| ATOM | 522 | C | ILE | 63 | −10.323 | 17.651 | 0.425 | 1.00 | 0.16 | 1SG | 523 |
| ATOM | 523 | O | ILE | 63 | −9.105 | 17.703 | 0.586 | 1.00 | 0.16 | 1SG | 524 |
| ATOM | 524 | N | VAL | 64 | −11.061 | 18.749 | 0.166 | 1.00 | 0.25 | 1SG | 525 |
| ATOM | 525 | CA | VAL | 64 | −10.413 | 20.009 | −0.067 | 1.00 | 0.25 | 1SG | 526 |
| ATOM | 526 | CB | VAL | 64 | −11.025 | 20.801 | −1.188 | 1.00 | 0.25 | 1SG | 527 |
| ATOM | 527 | CG1 | VAL | 64 | −10.926 | 20.008 | −2.499 | 1.00 | 0.25 | 1SG | 528 |
| ATOM | 528 | CG2 | VAL | 64 | −12.467 | 21.161 | −0.789 | 1.00 | 0.25 | 1SG | 529 |
| ATOM | 529 | C | VAL | 64 | −10.570 | 20.889 | 1.128 | 1.00 | 0.25 | 1SG | 530 |
| ATOM | 530 | O | VAL | 64 | −11.565 | 20.817 | 1.849 | 1.00 | 0.25 | 1SG | 531 |
| ATOM | 531 | N | GLU | 65 | −9.551 | 21.735 | 1.386 | 1.00 | 0.36 | 1SG | 532 |
| ATOM | 532 | CA | GLU | 65 | −9.719 | 22.722 | 2.410 | 1.00 | 0.36 | 1SG | 533 |
| ATOM | 533 | CB | GLU | 65 | −8.765 | 22.614 | 3.618 | 1.00 | 0.36 | 1SG | 534 |
| ATOM | 534 | CG | GLU | 65 | −7.397 | 23.253 | 3.459 | 1.00 | 0.36 | 1SG | 535 |
| ATOM | 535 | CD | GLU | 65 | −7.531 | 24.695 | 3.926 | 1.00 | 0.36 | 1SG | 536 |
| ATOM | 536 | OE1 | GLU | 65 | −8.656 | 25.078 | 4.343 | 1.00 | 0.36 | 1SG | 537 |
| ATOM | 537 | OE2 | GLU | 65 | −6.514 | 25.436 | 3.877 | 1.00 | 0.36 | 1SG | 538 |
| ATOM | 538 | C | GLU | 65 | −9.557 | 24.032 | 1.696 | 1.00 | 0.36 | 1SG | 539 |
| ATOM | 539 | O | GLU | 65 | −8.958 | 24.085 | 0.623 | 1.00 | 0.36 | 1SG | 540 |
| ATOM | 540 | N | PRO | 66 | −10.116 | 25.085 | 2.218 | 1.00 | 0.39 | 1SG | 541 |
| ATOM | 541 | CA | PRO | 66 | −10.056 | 26.332 | 1.502 | 1.00 | 0.39 | 1SG | 542 |
| ATOM | 542 | CD | PRO | 66 | −11.364 | 24.950 | 2.952 | 1.00 | 0.39 | 1SG | 543 |
| ATOM | 543 | CB | PRO | 66 | −11.117 | 27.229 | 2.135 | 1.00 | 0.39 | 1SG | 544 |
| ATOM | 544 | CG | PRO | 66 | −12.167 | 26.227 | 2.648 | 1.00 | 0.39 | 1SG | 545 |
| ATOM | 545 | C | PRO | 66 | −8.696 | 26.951 | 1.417 | 1.00 | 0.39 | 1SG | 546 |
| ATOM | 546 | O | PRO | 66 | −7.954 | 26.931 | 2.396 | 1.00 | 0.39 | 1SG | 547 |
| ATOM | 547 | N | LEU | 67 | −8.375 | 27.522 | 0.238 | 1.00 | 0.38 | 1SG | 548 |
| ATOM | 548 | CA | LEU | 67 | −7.116 | 28.143 | −0.060 | 1.00 | 0.38 | 1SG | 549 |
| ATOM | 549 | CB | LEU | 67 | −6.974 | 28.526 | −1.543 | 1.00 | 0.38 | 1SG | 550 |
| ATOM | 550 | CG | LEU | 67 | −6.937 | 27.318 | −2.495 | 1.00 | 0.38 | 1SG | 551 |
| ATOM | 551 | CD2 | LEU | 67 | −6.539 | 27.743 | −3.917 | 1.00 | 0.38 | 1SG | 552 |
| ATOM | 552 | CD1 | LEU | 67 | −8.255 | 26.530 | −2.457 | 1.00 | 0.38 | 1SG | 553 |
| ATOM | 553 | C | LEU | 67 | −6.956 | 29.405 | 0.725 | 1.00 | 0.38 | 1SG | 554 |
| ATOM | 554 | O | LEU | 67 | −5.840 | 29.745 | 1.121 | 1.00 | 0.38 | 1SG | 555 |
| ATOM | 555 | N | THR | 68 | −8.076 | 30.122 | 0.960 | 1.00 | 0.39 | 1SG | 556 |
| ATOM | 556 | CA | THR | 68 | −8.082 | 31.412 | 1.595 | 1.00 | 0.39 | 1SG | 557 |
| ATOM | 557 | CB | THR | 68 | −9.449 | 31.859 | 2.022 | 1.00 | 0.39 | 1SG | 558 |
| ATOM | 558 | OG1 | THR | 68 | −10.314 | 31.934 | 0.898 | 1.00 | 0.39 | 1SG | 559 |
| ATOM | 559 | CG2 | THR | 68 | −9.329 | 33.240 | 2.690 | 1.00 | 0.39 | 1SG | 560 |
| ATOM | 560 | C | THR | 68 | −7.212 | 31.390 | 2.807 | 1.00 | 0.39 | 1SG | 561 |
| ATOM | 561 | O | THR | 68 | −7.142 | 30.398 | 3.530 | 1.00 | 0.39 | 1SG | 562 |
| ATOM | 562 | N | LYS | 69 | −6.502 | 32.510 | 3.039 | 1.00 | 0.50 | 1SG | 563 |
| ATOM | 563 | CA | LYS | 69 | −5.584 | 32.594 | 4.132 | 1.00 | 0.50 | 1SG | 564 |
| ATOM | 564 | CB | LYS | 69 | −4.195 | 33.095 | 3.708 | 1.00 | 0.50 | 1SG | 565 |
| ATOM | 565 | CG | LYS | 69 | −4.231 | 34.436 | 2.976 | 1.00 | 0.50 | 1SG | 566 |
| ATOM | 566 | CD | LYS | 69 | −2.844 | 35.034 | 2.745 | 1.00 | 0.50 | 1SG | 567 |
| ATOM | 567 | CE | LYS | 69 | −2.082 | 35.323 | 4.039 | 1.00 | 0.50 | 1SG | 568 |
| ATOM | 568 | NZ | LYS | 69 | −0.752 | 35.895 | 3.726 | 1.00 | 0.50 | 1SG | 569 |
| ATOM | 569 | C | LYS | 69 | −6.151 | 33.531 | 5.142 | 1.00 | 0.50 | 1SG | 570 |
| ATOM | 570 | O | LYS | 69 | −7.116 | 34.244 | 4.874 | 1.00 | 0.50 | 1SG | 571 |
| ATOM | 571 | N | GLU | 70 | −5.577 | 33.518 | 6.360 | 1.00 | 0.55 | 1SG | 572 |
| ATOM | 572 | CA | GLU | 70 | −6.080 | 34.357 | 7.404 | 1.00 | 0.55 | 1SG | 573 |
| ATOM | 573 | CB | GLU | 70 | −6.463 | 33.584 | 8.670 | 1.00 | 0.55 | 1SG | 574 |
| ATOM | 574 | CG | GLU | 70 | −5.259 | 32.891 | 9.299 | 1.00 | 0.55 | 1SG | 575 |
| ATOM | 575 | CD | GLU | 70 | −4.849 | 31.719 | 8.417 | 1.00 | 0.55 | 1SG | 576 |
| ATOM | 576 | OE1 | GLU | 70 | −5.723 | 30.855 | 8.138 | 1.00 | 0.55 | 1SG | 577 |
| ATOM | 577 | OE2 | GLU | 70 | −3.657 | 31.675 | 8.010 | 1.00 | 0.55 | 1SG | 578 |
| ATOM | 578 | C | GLU | 70 | −5.000 | 35.317 | 7.786 | 1.00 | 0.55 | 1SG | 579 |
| ATOM | 579 | O | GLU | 70 | −3.816 | 35.060 | 7.572 | 1.00 | 0.55 | 1SG | 580 |
| ATOM | 580 | N | GLU | 71 | −5.399 | 36.469 | 8.357 | 1.00 | 0.60 | 1SG | 581 |
| ATOM | 581 | CA | GLU | 71 | −4.441 | 37.456 | 8.759 | 1.00 | 0.60 | 1SG | 582 |
| ATOM | 582 | CB | GLU | 71 | −4.957 | 38.894 | 8.560 | 1.00 | 0.60 | 1SG | 583 |
| ATOM | 583 | CG | GLU | 71 | −3.971 | 39.984 | 8.978 | 1.00 | 0.60 | 1SG | 584 |
| ATOM | 584 | CD | GLU | 71 | −4.245 | 40.328 | 10.436 | 1.00 | 0.60 | 1SG | 585 |
| ATOM | 585 | OE1 | GLU | 71 | −5.384 | 40.061 | 10.903 | 1.00 | 0.60 | 1SG | 586 |
| ATOM | 586 | OE2 | GLU | 71 | −3.321 | 40.867 | 11.100 | 1.00 | 0.60 | 1SG | 587 |
| ATOM | 587 | C | GLU | 71 | −4.169 | 37.243 | 10.214 | 1.00 | 0.60 | 1SG | 588 |
| ATOM | 588 | O | GLU | 71 | −5.081 | 37.278 | 11.039 | 1.00 | 0.60 | 1SG | 589 |
| ATOM | 589 | N | VAL | 72 | −2.886 | 37.002 | 10.562 | 1.00 | 0.56 | 1SG | 590 |
| ATOM | 590 | CA | VAL | 72 | −2.527 | 36.744 | 11.927 | 1.00 | 0.56 | 1SG | 591 |
| ATOM | 591 | CB | VAL | 72 | −2.053 | 35.340 | 12.162 | 1.00 | 0.56 | 1SG | 592 |
| ATOM | 592 | CG1 | VAL | 72 | −3.211 | 34.375 | 11.859 | 1.00 | 0.56 | 1SG | 593 |
| ATOM | 593 | CG2 | VAL | 72 | −0.798 | 35.099 | 11.305 | 1.00 | 0.56 | 1SG | 594 |
| ATOM | 594 | C | VAL | 72 | −1.393 | 37.644 | 12.280 | 1.00 | 0.56 | 1SG | 595 |
| ATOM | 595 | O | VAL | 72 | −0.968 | 38.469 | 11.471 | 1.00 | 0.56 | 1SG | 596 |
| ATOM | 596 | N | GLY | 73 | −0.914 | 37.570 | 13.540 | 1.00 | 0.39 | 1SG | 597 |
| ATOM | 597 | CA | GLY | 73 | 0.167 | 38.459 | 13.815 | 1.00 | 0.39 | 1SG | 598 |
| ATOM | 598 | C | GLY | 73 | 1.369 | 38.081 | 13.002 | 1.00 | 0.39 | 1SG | 599 |
| ATOM | 599 | O | GLY | 73 | 1.603 | 38.667 | 11.945 | 1.00 | 0.39 | 1SG | 600 |
| ATOM | 600 | N | PHE | 74 | 2.217 | 37.150 | 13.514 | 1.00 | 0.50 | 1SG | 601 |
| ATOM | 601 | CA | PHE | 74 | 3.345 | 36.764 | 12.711 | 1.00 | 0.50 | 1SG | 602 |
| ATOM | 602 | CB | PHE | 74 | 4.681 | 36.948 | 13.454 | 1.00 | 0.50 | 1SG | 603 |
| ATOM | 603 | CG | PHE | 74 | 4.757 | 38.408 | 13.727 | 1.00 | 0.50 | 1SG | 604 |
| ATOM | 604 | CD1 | PHE | 74 | 5.213 | 39.276 | 12.762 | 1.00 | 0.50 | 1SG | 605 |
| ATOM | 605 | CD2 | PHE | 74 | 4.358 | 38.908 | 14.944 | 1.00 | 0.50 | 1SG | 606 |
| ATOM | 606 | CE1 | PHE | 74 | 5.278 | 40.625 | 13.013 | 1.00 | 0.50 | 1SG | 607 |
| ATOM | 607 | CE2 | PHE | 74 | 4.421 | 40.257 | 15.200 | 1.00 | 0.50 | 1SG | 608 |
| ATOM | 608 | CZ | PHE | 74 | 4.882 | 41.118 | 14.233 | 1.00 | 0.50 | 1SG | 609 |
| ATOM | 609 | C | PHE | 74 | 3.298 | 35.425 | 12.011 | 1.00 | 0.50 | 1SG | 610 |
| ATOM | 610 | O | PHE | 74 | 3.288 | 35.370 | 10.781 | 1.00 | 0.50 | 1SG | 611 |
| ATOM | 611 | N | PRO | 75 | 3.196 | 34.315 | 12.725 | 1.00 | 0.80 | 1SG | 612 |
| ATOM | 612 | CA | PRO | 75 | 3.364 | 33.070 | 12.022 | 1.00 | 0.80 | 1SG | 613 |
| ATOM | 613 | CD | PRO | 75 | 3.655 | 34.200 | 14.104 | 1.00 | 0.80 | 1SG | 614 |
| ATOM | 614 | CB | PRO | 75 | 3.718 | 32.011 | 13.063 | 1.00 | 0.80 | 1SG | 615 |
| ATOM | 615 | CG | PRO | 75 | 4.334 | 32.823 | 14.211 | 1.00 | 0.80 | 1SG | 616 |
| ATOM | 616 | C | PRO | 75 | 2.150 | 32.717 | 11.254 | 1.00 | 0.80 | 1SG | 617 |
| ATOM | 617 | O | PRO | 75 | 1.125 | 32.458 | 11.880 | 1.00 | 0.80 | 1SG | 618 |
| ATOM | 618 | N | ILE | 76 | 2.234 | 32.647 | 9.915 | 1.00 | 0.70 | 1SG | 619 |
| ATOM | 619 | CA | ILE | 76 | 1.044 | 32.274 | 9.222 | 1.00 | 0.70 | 1SG | 620 |
| ATOM | 620 | CB | ILE | 76 | 1.035 | 32.590 | 7.747 | 1.00 | 0.70 | 1SG | 621 |
| ATOM | 621 | CG2 | ILE | 76 | 1.365 | 34.082 | 7.617 | 1.00 | 0.70 | 1SG | 622 |
| ATOM | 622 | CG1 | ILE | 76 | 1.963 | 31.692 | 6.918 | 1.00 | 0.70 | 1SG | 623 |
| ATOM | 623 | CD1 | ILE | 76 | 1.317 | 30.358 | 6.539 | 1.00 | 0.70 | 1SG | 624 |
| ATOM | 624 | C | ILE | 76 | 0.885 | 30.804 | 9.409 | 1.00 | 0.70 | 1SG | 625 |
| ATOM | 625 | O | ILE | 76 | −0.226 | 30.289 | 9.492 | 1.00 | 0.70 | 1SG | 626 |
| ATOM | 626 | N | ALA | 77 | 2.029 | 30.097 | 9.491 | 1.00 | 0.38 | 1SG | 627 |
| ATOM | 627 | CA | ALA | 77 | 2.066 | 28.668 | 9.543 | 1.00 | 0.38 | 1SG | 628 |
| ATOM | 628 | CB | ALA | 77 | 3.502 | 28.130 | 9.604 | 1.00 | 0.38 | 1SG | 629 |
| ATOM | 629 | C | ALA | 77 | 1.328 | 28.175 | 10.746 | 1.00 | 0.38 | 1SG | 630 |
| ATOM | 630 | O | ALA | 77 | 0.573 | 27.210 | 10.655 | 1.00 | 0.38 | 1SG | 631 |
| ATOM | 631 | N | TYR | 78 | 1.515 | 28.815 | 11.914 | 1.00 | 0.36 | 1SG | 632 |
| ATOM | 632 | CA | TYR | 78 | 0.842 | 28.328 | 13.083 | 1.00 | 0.36 | 1SG | 633 |
| ATOM | 633 | CB | TYR | 78 | 1.199 | 29.078 | 14.381 | 1.00 | 0.36 | 1SG | 634 |
| ATOM | 634 | CG | TYR | 78 | 2.569 | 28.665 | 14.790 | 1.00 | 0.36 | 1SG | 635 |
| ATOM | 635 | CD1 | TYR | 78 | 2.798 | 27.365 | 15.163 | 1.00 | 0.36 | 1SG | 636 |
| ATOM | 636 | CD2 | TYR | 78 | 3.610 | 29.559 | 14.843 | 1.00 | 0.36 | 1SG | 637 |
| ATOM | 637 | CE1 | TYR | 78 | 4.048 | 26.950 | 15.553 | 1.00 | 0.36 | 1SG | 638 |
| ATOM | 638 | CE2 | TYR | 78 | 4.865 | 29.151 | 15.234 | 1.00 | 0.36 | 1SG | 639 |
| ATOM | 639 | CZ | TYR | 78 | 5.088 | 27.843 | 15.587 | 1.00 | 0.36 | 1SG | 640 |
| ATOM | 640 | OH | TYR | 78 | 6.373 | 27.421 | 15.988 | 1.00 | 0.36 | 1SG | 641 |
| ATOM | 641 | C | TYR | 78 | −0.634 | 28.450 | 12.902 | 1.00 | 0.36 | 1SG | 642 |
| ATOM | 642 | O | TYR | 78 | −1.397 | 27.564 | 13.275 | 1.00 | 0.36 | 1SG | 643 |
| ATOM | 643 | N | SER | 79 | −1.088 | 29.569 | 12.330 | 1.00 | 0.51 | 1SG | 644 |
| ATOM | 644 | CA | SER | 79 | −2.493 | 29.770 | 12.165 | 1.00 | 0.51 | 1SG | 645 |
| ATOM | 645 | CB | SER | 79 | −2.787 | 31.130 | 11.543 | 1.00 | 0.51 | 1SG | 646 |
| ATOM | 646 | OG | SER | 79 | −4.134 | 31.135 | 11.122 | 1.00 | 0.51 | 1SG | 647 |
| ATOM | 647 | C | SER | 79 | −3.031 | 28.744 | 11.228 | 1.00 | 0.51 | 1SG | 648 |
| ATOM | 648 | O | SER | 79 | −4.072 | 28.134 | 11.470 | 1.00 | 0.51 | 1SG | 649 |
| ATOM | 649 | N | ILE | 80 | −2.294 | 28.521 | 10.132 | 1.00 | 0.54 | 1SG | 650 |
| ATOM | 650 | CA | ILE | 80 | −2.694 | 27.616 | 9.106 | 1.00 | 0.54 | 1SG | 651 |
| ATOM | 651 | CB | ILE | 80 | −1.720 | 27.599 | 7.960 | 1.00 | 0.54 | 1SG | 652 |
| ATOM | 652 | CG2 | ILE | 80 | −2.023 | 26.385 | 7.094 | 1.00 | 0.54 | 1SG | 653 |
| ATOM | 653 | CG1 | ILE | 80 | −1.814 | 28.886 | 7.138 | 1.00 | 0.54 | 1SG | 654 |
| ATOM | 654 | CD1 | ILE | 80 | −3.149 | 29.018 | 6.403 | 1.00 | 0.54 | 1SG | 655 |
| ATOM | 655 | C | ILE | 80 | −2.784 | 26.245 | 9.685 | 1.00 | 0.54 | 1SG | 656 |
| ATOM | 656 | O | ILE | 80 | −3.611 | 25.449 | 9.245 | 1.00 | 0.54 | 1SG | 657 |
| ATOM | 657 | N | VAL | 81 | −1.946 | 25.920 | 10.689 | 1.00 | 0.32 | 1SG | 658 |
| ATOM | 658 | CA | VAL | 81 | −1.958 | 24.555 | 11.127 | 1.00 | 0.32 | 1SG | 659 |
| ATOM | 659 | CB | VAL | 81 | −0.915 | 24.212 | 12.176 | 1.00 | 0.32 | 1SG | 660 |
| ATOM | 660 | CG1 | VAL | 81 | 0.488 | 24.528 | 11.638 | 1.00 | 0.32 | 1SG | 661 |
| ATOM | 661 | CG2 | VAL | 81 | −1.237 | 24.851 | 13.532 | 1.00 | 0.32 | 1SG | 662 |
| ATOM | 662 | C | VAL | 81 | −3.320 | 24.192 | 11.644 | 1.00 | 0.32 | 1SG | 663 |
| ATOM | 663 | O | VAL | 81 | −3.908 | 23.205 | 11.203 | 1.00 | 0.32 | 1SG | 664 |
| ATOM | 664 | N | VAL | 82 | −3.879 | 24.980 | 12.579 | 1.00 | 0.20 | 1SG | 665 |
| ATOM | 665 | CA | VAL | 82 | −5.184 | 24.663 | 13.083 | 1.00 | 0.20 | 1SG | 666 |
| ATOM | 666 | CB | VAL | 82 | −5.523 | 25.414 | 14.337 | 1.00 | 0.20 | 1SG | 667 |
| ATOM | 667 | CG1 | VAL | 82 | −6.940 | 25.010 | 14.779 | 1.00 | 0.20 | 1SG | 668 |
| ATOM | 668 | CG2 | VAL | 82 | −4.439 | 25.124 | 15.389 | 1.00 | 0.20 | 1SG | 669 |
| ATOM | 669 | C | VAL | 82 | −6.237 | 24.996 | 12.069 | 1.00 | 0.20 | 1SG | 670 |
| ATOM | 670 | O | VAL | 82 | −7.108 | 24.179 | 11.777 | 1.00 | 0.20 | 1SG | 671 |
| ATOM | 671 | N | HIS | 83 | −6.164 | 26.214 | 11.492 | 1.00 | 0.32 | 1SG | 672 |
| ATOM | 672 | CA | HIS | 83 | −7.207 | 26.704 | 10.631 | 1.00 | 0.32 | 1SG | 673 |
| ATOM | 673 | ND1 | HIS | 83 | −9.124 | 29.371 | 9.924 | 1.00 | 0.32 | 1SG | 674 |
| ATOM | 674 | CG | HIS | 83 | −8.013 | 28.780 | 9.366 | 1.00 | 0.32 | 1SG | 675 |
| ATOM | 675 | CB | HIS | 83 | −6.914 | 28.143 | 10.164 | 1.00 | 0.32 | 1SG | 676 |
| ATOM | 676 | NE2 | HIS | 83 | −9.324 | 29.599 | 7.721 | 1.00 | 0.32 | 1SG | 677 |
| ATOM | 677 | CD2 | HIS | 83 | −8.152 | 28.927 | 8.019 | 1.00 | 0.32 | 1SG | 678 |
| ATOM | 678 | CE1 | HIS | 83 | −9.873 | 29.844 | 8.897 | 1.00 | 0.32 | 1SG | 679 |
| ATOM | 679 | C | HIS | 83 | −7.336 | 25.854 | 9.406 | 1.00 | 0.32 | 1SG | 680 |
| ATOM | 680 | O | HIS | 83 | −8.373 | 25.240 | 9.164 | 1.00 | 0.32 | 1SG | 681 |
| ATOM | 681 | N | HIS | 84 | −6.252 | 25.801 | 8.615 | 1.00 | 0.48 | 1SG | 682 |
| ATOM | 682 | CA | HIS | 84 | −6.134 | 25.060 | 7.395 | 1.00 | 0.48 | 1SG | 683 |
| ATOM | 683 | ND1 | HIS | 84 | −3.928 | 23.140 | 6.017 | 1.00 | 0.48 | 1SG | 684 |
| ATOM | 684 | CG | HIS | 84 | −4.061 | 24.501 | 5.844 | 1.00 | 0.48 | 1SG | 685 |
| ATOM | 685 | CB | HIS | 84 | −4.776 | 25.429 | 6.781 | 1.00 | 0.48 | 1SG | 686 |
| ATOM | 686 | NE2 | HIS | 84 | −2.641 | 23.684 | 4.299 | 1.00 | 0.48 | 1SG | 687 |
| ATOM | 687 | CD2 | HIS | 84 | −3.273 | 24.813 | 4.782 | 1.00 | 0.48 | 1SG | 688 |
| ATOM | 688 | CE1 | HIS | 84 | −3.069 | 22.703 | 5.067 | 1.00 | 0.48 | 1SG | 689 |
| ATOM | 689 | C | HIS | 84 | −6.171 | 23.611 | 7.700 | 1.00 | 0.48 | 1SG | 690 |
| ATOM | 690 | O | HIS | 84 | −7.056 | 22.898 | 7.231 | 1.00 | 0.48 | 1SG | 691 |
| ATOM | 691 | N | LYS | 85 | −5.315 | 23.214 | 8.658 | 1.00 | 0.70 | 1SG | 692 |
| ATOM | 692 | CA | LYS | 85 | −4.965 | 21.865 | 8.986 | 1.00 | 0.70 | 1SG | 693 |
| ATOM | 693 | CB | LYS | 85 | −5.778 | 20.746 | 8.305 | 1.00 | 0.70 | 1SG | 694 |
| ATOM | 694 | CG | LYS | 85 | −7.238 | 20.612 | 8.731 | 1.00 | 0.70 | 1SG | 695 |
| ATOM | 695 | CD | LYS | 85 | −8.038 | 19.730 | 7.768 | 1.00 | 0.70 | 1SG | 696 |
| ATOM | 696 | CE | LYS | 85 | −8.062 | 20.262 | 6.331 | 1.00 | 0.70 | 1SG | 697 |
| ATOM | 697 | NZ | LYS | 85 | −6.762 | 20.015 | 5.669 | 1.00 | 0.70 | 1SG | 698 |
| ATOM | 698 | C | LYS | 85 | −3.566 | 21.597 | 8.533 | 1.00 | 0.70 | 1SG | 699 |
| ATOM | 699 | O | LYS | 85 | −3.254 | 20.431 | 8.287 | 1.00 | 0.70 | 1SG | 700 |
| ATOM | 700 | N | ILE | 86 | −2.670 | 22.614 | 8.401 | 1.00 | 0.79 | 1SG | 701 |
| ATOM | 701 | CA | ILE | 86 | −1.357 | 22.107 | 8.108 | 1.00 | 0.79 | 1SG | 702 |
| ATOM | 702 | CB | ILE | 86 | −0.171 | 22.959 | 7.681 | 1.00 | 0.79 | 1SG | 703 |
| ATOM | 703 | CG2 | ILE | 86 | −0.665 | 23.831 | 6.528 | 1.00 | 0.79 | 1SG | 704 |
| ATOM | 704 | CG1 | ILE | 86 | 0.516 | 23.800 | 8.759 | 1.00 | 0.79 | 1SG | 705 |
| ATOM | 705 | CD1 | ILE | 86 | 0.035 | 25.233 | 8.825 | 1.00 | 0.79 | 1SG | 706 |
| ATOM | 706 | C | ILE | 86 | −0.943 | 21.393 | 9.347 | 1.00 | 0.79 | 1SG | 707 |
| ATOM | 707 | O | ILE | 86 | −0.136 | 20.468 | 9.292 | 1.00 | 0.79 | 1SG | 708 |
| ATOM | 708 | N | GLU | 87 | −1.440 | 21.849 | 10.515 | 1.00 | 0.83 | 1SG | 709 |
| ATOM | 709 | CA | GLU | 87 | −1.245 | 21.012 | 11.657 | 1.00 | 0.83 | 1SG | 710 |
| ATOM | 710 | CB | GLU | 87 | −1.816 | 21.545 | 12.980 | 1.00 | 0.83 | 1SG | 711 |
| ATOM | 711 | CG | GLU | 87 | −1.377 | 20.722 | 14.192 | 1.00 | 0.83 | 1SG | 712 |
| ATOM | 712 | CD | GLU | 87 | 0.072 | 21.082 | 14.488 | 1.00 | 0.83 | 1SG | 713 |
| ATOM | 713 | OE1 | GLU | 87 | 0.321 | 22.243 | 14.909 | 1.00 | 0.83 | 1SG | 714 |
| ATOM | 714 | OE2 | GLU | 87 | 0.950 | 20.200 | 14.290 | 1.00 | 0.83 | 1SG | 715 |
| ATOM | 715 | C | GLU | 87 | −2.070 | 19.812 | 11.340 | 1.00 | 0.83 | 1SG | 716 |
| ATOM | 716 | O | GLU | 87 | −1.541 | 18.702 | 11.325 | 1.00 | 0.83 | 1SG | 717 |
| ATOM | 717 | N | MET | 88 | −3.328 | 20.078 | 10.882 | 1.00 | 0.87 | 1SG | 718 |
| ATOM | 718 | CA | MET | 88 | −4.371 | 19.128 | 10.545 | 1.00 | 0.87 | 1SG | 719 |
| ATOM | 719 | CB | MET | 88 | −3.981 | 17.658 | 10.775 | 1.00 | 0.87 | 1SG | 720 |
| ATOM | 720 | CG | MET | 88 | −4.215 | 17.164 | 12.207 | 1.00 | 0.87 | 1SG | 721 |
| ATOM | 721 | SD | MET | 88 | −3.273 | 18.005 | 13.509 | 1.00 | 0.87 | 1SG | 722 |
| ATOM | 722 | CE | MET | 88 | −4.540 | 17.810 | 14.790 | 1.00 | 0.87 | 1SG | 723 |
| ATOM | 723 | C | MET | 88 | −5.603 | 19.240 | 11.404 | 1.00 | 0.87 | 1SG | 724 |
| ATOM | 724 | O | MET | 88 | −6.488 | 18.392 | 11.300 | 1.00 | 0.87 | 1SG | 725 |
| ATOM | 725 | N | LEU | 89 | −5.726 | 20.260 | 12.265 | 1.00 | 0.60 | 1SG | 726 |
| ATOM | 726 | CA | LEU | 89 | −6.818 | 20.234 | 13.204 | 1.00 | 0.60 | 1SG | 727 |
| ATOM | 727 | CB | LEU | 89 | −6.617 | 21.276 | 14.316 | 1.00 | 0.60 | 1SG | 728 |
| ATOM | 728 | CG | LEU | 89 | −7.845 | 21.450 | 15.223 | 1.00 | 0.60 | 1SG | 729 |
| ATOM | 729 | CD2 | LEU | 89 | −7.602 | 22.549 | 16.266 | 1.00 | 0.60 | 1SG | 730 |
| ATOM | 730 | CD1 | LEU | 89 | −8.270 | 20.122 | 15.859 | 1.00 | 0.60 | 1SG | 731 |
| ATOM | 731 | C | LEU | 89 | −8.254 | 20.386 | 12.738 | 1.00 | 0.60 | 1SG | 732 |
| ATOM | 732 | O | LEU | 89 | −9.027 | 19.429 | 12.788 | 1.00 | 0.60 | 1SG | 733 |
| ATOM | 733 | N | ASP | 90 | −8.631 | 21.585 | 12.247 | 1.00 | 0.52 | 1SG | 734 |
| ATOM | 734 | CA | ASP | 90 | −10.026 | 21.965 | 12.167 | 1.00 | 0.52 | 1SG | 735 |
| ATOM | 735 | CB | ASP | 90 | −10.171 | 23.479 | 11.880 | 1.00 | 0.52 | 1SG | 736 |
| ATOM | 736 | CG | ASP | 90 | −11.615 | 23.965 | 12.048 | 1.00 | 0.52 | 1SG | 737 |
| ATOM | 737 | OD1 | ASP | 90 | −12.533 | 23.464 | 11.346 | 1.00 | 0.52 | 1SG | 738 |
| ATOM | 738 | OD2 | ASP | 90 | −11.811 | 24.869 | 12.905 | 1.00 | 0.52 | 1SG | 739 |
| ATOM | 739 | C | ASP | 90 | −10.920 | 21.268 | 11.190 | 1.00 | 0.52 | 1SG | 740 |
| ATOM | 740 | O | ASP | 90 | −11.811 | 20.512 | 11.579 | 1.00 | 0.52 | 1SG | 741 |
| ATOM | 741 | N | ARG | 91 | −10.698 | 21.499 | 9.886 | 1.00 | 0.73 | 1SG | 742 |
| ATOM | 742 | CA | ARG | 91 | −11.706 | 21.082 | 8.956 | 1.00 | 0.73 | 1SG | 743 |
| ATOM | 743 | CB | ARG | 91 | −11.714 | 21.849 | 7.622 | 1.00 | 0.73 | 1SG | 744 |
| ATOM | 744 | CG | ARG | 91 | −10.365 | 21.999 | 6.943 | 1.00 | 0.73 | 1SG | 745 |
| ATOM | 745 | CD | ARG | 91 | −9.805 | 23.403 | 7.175 | 1.00 | 0.73 | 1SG | 746 |
| ATOM | 746 | NE | ARG | 91 | −10.783 | 24.363 | 6.584 | 1.00 | 0.73 | 1SG | 747 |
| ATOM | 747 | CZ | ARG | 91 | −11.829 | 24.845 | 7.319 | 1.00 | 0.73 | 1SG | 748 |
| ATOM | 748 | NH1 | ARG | 91 | −11.961 | 24.515 | 8.637 | 1.00 | 0.73 | 1SG | 749 |
| ATOM | 749 | NH2 | ARG | 91 | −12.750 | 25.663 | 6.730 | 1.00 | 0.73 | 1SG | 750 |
| ATOM | 750 | C | ARG | 91 | −11.829 | 19.610 | 8.745 | 1.00 | 0.73 | 1SG | 751 |
| ATOM | 751 | O | ARG | 91 | −12.942 | 19.108 | 8.609 | 1.00 | 0.73 | 1SG | 752 |
| ATOM | 752 | N | LEU | 92 | −10.717 | 18.863 | 8.718 | 1.00 | 0.67 | 1SG | 753 |
| ATOM | 753 | CA | LEU | 92 | −10.848 | 17.471 | 8.388 | 1.00 | 0.67 | 1SG | 754 |
| ATOM | 754 | CB | LEU | 92 | −9.473 | 16.764 | 8.255 | 1.00 | 0.67 | 1SG | 755 |
| ATOM | 755 | CG | LEU | 92 | −9.446 | 15.291 | 7.745 | 1.00 | 0.67 | 1SG | 756 |
| ATOM | 756 | CD2 | LEU | 92 | −10.298 | 14.308 | 8.567 | 1.00 | 0.67 | 1SG | 757 |
| ATOM | 757 | CD1 | LEU | 92 | −8.000 | 14.778 | 7.675 | 1.00 | 0.67 | 1SG | 758 |
| ATOM | 758 | C | LEU | 92 | −11.655 | 16.746 | 9.421 | 1.00 | 0.67 | 1SG | 759 |
| ATOM | 759 | O | LEU | 92 | −12.578 | 16.008 | 9.080 | 1.00 | 0.67 | 1SG | 760 |
| ATOM | 760 | N | LEU | 93 | −11.346 | 16.935 | 10.714 | 1.00 | 0.51 | 1SG | 761 |
| ATOM | 761 | CA | LEU | 93 | −12.019 | 16.139 | 11.704 | 1.00 | 0.51 | 1SG | 762 |
| ATOM | 762 | CB | LEU | 93 | −11.492 | 16.378 | 13.129 | 1.00 | 0.51 | 1SG | 763 |
| ATOM | 763 | CG | LEU | 93 | −12.004 | 15.357 | 14.164 | 1.00 | 0.51 | 1SG | 764 |
| ATOM | 764 | CD2 | LEU | 93 | −11.578 | 13.930 | 13.779 | 1.00 | 0.51 | 1SG | 765 |
| ATOM | 765 | CD1 | LEU | 93 | −13.515 | 15.482 | 14.409 | 1.00 | 0.51 | 1SG | 766 |
| ATOM | 766 | C | LEU | 93 | −13.460 | 16.512 | 11.649 | 1.00 | 0.51 | 1SG | 767 |
| ATOM | 767 | O | LEU | 93 | −14.353 | 15.679 | 11.803 | 1.00 | 0.51 | 1SG | 768 |
| ATOM | 768 | N | ARG | 94 | −13.686 | 17.803 | 11.374 | 1.00 | 0.33 | 1SG | 769 |
| ATOM | 769 | CA | ARG | 94 | −14.955 | 18.453 | 11.288 | 1.00 | 0.33 | 1SG | 770 |
| ATOM | 770 | CB | ARG | 94 | −14.735 | 19.916 | 10.857 | 1.00 | 0.33 | 1SG | 771 |
| ATOM | 771 | CG | ARG | 94 | −15.968 | 20.808 | 10.723 | 1.00 | 0.33 | 1SG | 772 |
| ATOM | 772 | CD | ARG | 94 | −15.573 | 22.223 | 10.289 | 1.00 | 0.33 | 1SG | 773 |
| ATOM | 773 | NE | ARG | 94 | −16.806 | 23.050 | 10.189 | 1.00 | 0.33 | 1SG | 774 |
| ATOM | 774 | CZ | ARG | 94 | −16.697 | 24.361 | 9.825 | 1.00 | 0.33 | 1SG | 775 |
| ATOM | 775 | NH1 | ARG | 94 | −15.470 | 24.879 | 9.521 | 1.00 | 0.33 | 1SG | 776 |
| ATOM | 776 | NH2 | ARG | 94 | −17.808 | 25.152 | 9.771 | 1.00 | 0.33 | 1SG | 777 |
| ATOM | 777 | C | ARG | 94 | −15.725 | 17.754 | 10.218 | 1.00 | 0.33 | 1SG | 778 |
| ATOM | 778 | O | ARG | 94 | −16.951 | 17.668 | 10.270 | 1.00 | 0.33 | 1SG | 779 |
| ATOM | 779 | N | ALA | 95 | −15.002 | 17.210 | 9.225 | 1.00 | 0.24 | 1SG | 780 |
| ATOM | 780 | CA | ALA | 95 | −15.635 | 16.568 | 8.115 | 1.00 | 0.24 | 1SG | 781 |
| ATOM | 781 | CB | ALA | 95 | −14.629 | 15.896 | 7.164 | 1.00 | 0.24 | 1SG | 782 |
| ATOM | 782 | C | ALA | 95 | −16.544 | 15.505 | 8.632 | 1.00 | 0.24 | 1SG | 783 |
| ATOM | 783 | O | ALA | 95 | −17.654 | 15.341 | 8.127 | 1.00 | 0.24 | 1SG | 784 |
| ATOM | 784 | N | ILE | 96 | −16.124 | 14.739 | 9.651 | 1.00 | 0.33 | 1SG | 785 |
| ATOM | 785 | CA | ILE | 96 | −17.059 | 13.732 | 10.033 | 1.00 | 0.33 | 1SG | 786 |
| ATOM | 786 | CB | ILE | 96 | −16.529 | 12.351 | 9.764 | 1.00 | 0.33 | 1SG | 787 |
| ATOM | 787 | CG2 | ILE | 96 | −15.239 | 12.114 | 10.571 | 1.00 | 0.33 | 1SG | 788 |
| ATOM | 788 | CG1 | ILE | 96 | −17.638 | 11.317 | 9.968 | 1.00 | 0.33 | 1SG | 789 |
| ATOM | 789 | CD1 | ILE | 96 | −17.326 | 9.985 | 9.301 | 1.00 | 0.33 | 1SG | 790 |
| ATOM | 790 | C | ILE | 96 | −17.490 | 13.884 | 11.468 | 1.00 | 0.33 | 1SG | 791 |
| ATOM | 791 | O | ILE | 96 | −16.997 | 13.200 | 12.363 | 1.00 | 0.33 | 1SG | 792 |
| ATOM | 792 | N | TYR | 97 | −18.492 | 14.761 | 11.704 | 1.00 | 0.42 | 1SG | 793 |
| ATOM | 793 | CA | TYR | 97 | −19.083 | 14.956 | 13.003 | 1.00 | 0.42 | 1SG | 794 |
| ATOM | 794 | CB | TYR | 97 | −19.934 | 16.232 | 13.152 | 1.00 | 0.42 | 1SG | 795 |
| ATOM | 795 | CG | TYR | 97 | −19.022 | 17.374 | 13.433 | 1.00 | 0.42 | 1SG | 796 |
| ATOM | 796 | CD1 | TYR | 97 | −18.576 | 17.570 | 14.719 | 1.00 | 0.42 | 1SG | 797 |
| ATOM | 797 | CD2 | TYR | 97 | −18.623 | 18.246 | 12.447 | 1.00 | 0.42 | 1SG | 798 |
| ATOM | 798 | CE1 | TYR | 97 | −17.736 | 18.611 | 15.025 | 1.00 | 0.42 | 1SG | 799 |
| ATOM | 799 | CE2 | TYR | 97 | −17.781 | 19.292 | 12.748 | 1.00 | 0.42 | 1SG | 800 |
| ATOM | 800 | CZ | TYR | 97 | −17.336 | 19.475 | 14.037 | 1.00 | 0.42 | 1SG | 801 |
| ATOM | 801 | OH | TYR | 97 | −16.473 | 20.546 | 14.349 | 1.00 | 0.42 | 1SG | 802 |
| ATOM | 802 | C | TYR | 97 | −19.984 | 13.796 | 13.192 | 1.00 | 0.42 | 1SG | 803 |
| ATOM | 803 | O | TYR | 97 | −19.498 | 12.665 | 13.146 | 1.00 | 0.42 | 1SG | 804 |
| ATOM | 804 | N | MET | 98 | −21.280 | 14.065 | 13.499 | 1.00 | 0.44 | 1SG | 805 |
| ATOM | 805 | CA | MET | 98 | −22.257 | 13.013 | 13.521 | 1.00 | 0.44 | 1SG | 806 |
| ATOM | 806 | CB | MET | 98 | −23.709 | 13.517 | 13.518 | 1.00 | 0.44 | 1SG | 807 |
| ATOM | 807 | CG | MET | 98 | −24.085 | 14.333 | 14.754 | 1.00 | 0.44 | 1SG | 808 |
| ATOM | 808 | SD | MET | 98 | −25.790 | 14.962 | 14.738 | 1.00 | 0.44 | 1SG | 809 |
| ATOM | 809 | CE | MET | 98 | −25.509 | 16.132 | 13.377 | 1.00 | 0.44 | 1SG | 810 |
| ATOM | 810 | C | MET | 98 | −22.001 | 12.390 | 12.201 | 1.00 | 0.44 | 1SG | 811 |
| ATOM | 811 | O | MET | 98 | −22.235 | 12.994 | 11.154 | 1.00 | 0.44 | 1SG | 812 |
| ATOM | 812 | N | PRO | 99 | −21.541 | 11.170 | 12.328 | 1.00 | 0.57 | 1SG | 813 |
| ATOM | 813 | CA | PRO | 99 | −20.819 | 10.492 | 11.284 | 1.00 | 0.57 | 1SG | 814 |
| ATOM | 814 | CD | PRO | 99 | −22.325 | 10.235 | 13.120 | 1.00 | 0.57 | 1SG | 815 |
| ATOM | 815 | CB | PRO | 99 | −21.042 | 8.994 | 11.494 | 1.00 | 0.57 | 1SG | 816 |
| ATOM | 816 | CG | PRO | 99 | −22.343 | 8.930 | 12.308 | 1.00 | 0.57 | 1SG | 817 |
| ATOM | 817 | C | PRO | 99 | −21.050 | 10.922 | 9.886 | 1.00 | 0.57 | 1SG | 818 |
| ATOM | 818 | O | PRO | 99 | −20.293 | 11.782 | 9.440 | 1.00 | 0.57 | 1SG | 819 |
| ATOM | 819 | N | GLN | 100 | −22.091 | 10.386 | 9.231 | 1.00 | 0.74 | 1SG | 820 |
| ATOM | 820 | CA | GLN | 100 | −22.443 | 10.634 | 7.863 | 1.00 | 0.74 | 1SG | 821 |
| ATOM | 821 | CB | GLN | 100 | −21.501 | 11.545 | 7.014 | 1.00 | 0.74 | 1SG | 822 |
| ATOM | 822 | CG | GLN | 100 | −20.039 | 11.116 | 6.801 | 1.00 | 0.74 | 1SG | 823 |
| ATOM | 823 | CD | GLN | 100 | −19.407 | 12.101 | 5.827 | 1.00 | 0.74 | 1SG | 824 |
| ATOM | 824 | OE1 | GLN | 100 | −20.034 | 13.081 | 5.428 | 1.00 | 0.74 | 1SG | 825 |
| ATOM | 825 | NE2 | GLN | 100 | −18.133 | 11.835 | 5.432 | 1.00 | 0.74 | 1SG | 826 |
| ATOM | 826 | C | GLN | 100 | −22.590 | 9.285 | 7.237 | 1.00 | 0.74 | 1SG | 827 |
| ATOM | 827 | O | GLN | 100 | −23.454 | 8.504 | 7.632 | 1.00 | 0.74 | 1SG | 828 |
| ATOM | 828 | N | ASN | 101 | −21.748 | 8.978 | 6.235 | 1.00 | 0.64 | 1SG | 829 |
| ATOM | 829 | CA | ASN | 101 | −21.812 | 7.741 | 5.520 | 1.00 | 0.64 | 1SG | 830 |
| ATOM | 830 | CB | ASN | 101 | −20.990 | 7.759 | 4.223 | 1.00 | 0.64 | 1SG | 831 |
| ATOM | 831 | CG | ASN | 101 | −21.649 | 8.735 | 3.264 | 1.00 | 0.64 | 1SG | 832 |
| ATOM | 832 | OD1 | ASN | 101 | −22.847 | 8.656 | 2.994 | 1.00 | 0.64 | 1SG | 833 |
| ATOM | 833 | ND2 | ASN | 101 | −20.841 | 9.693 | 2.737 | 1.00 | 0.64 | 1SG | 834 |
| ATOM | 834 | C | ASN | 101 | −21.271 | 6.619 | 6.349 | 1.00 | 0.64 | 1SG | 835 |
| ATOM | 835 | O | ASN | 101 | −21.974 | 5.646 | 6.606 | 1.00 | 0.64 | 1SG | 836 |
| ATOM | 836 | N | PHE | 102 | −19.993 | 6.692 | 6.770 | 1.00 | 0.51 | 1SG | 837 |
| ATOM | 837 | CA | PHE | 102 | −19.463 | 5.589 | 7.522 | 1.00 | 0.51 | 1SG | 838 |
| ATOM | 838 | CB | PHE | 102 | −18.801 | 4.520 | 6.639 | 1.00 | 0.51 | 1SG | 839 |
| ATOM | 839 | CG | PHE | 102 | −17.828 | 5.246 | 5.785 | 1.00 | 0.51 | 1SG | 840 |
| ATOM | 840 | CD1 | PHE | 102 | −16.599 | 5.625 | 6.272 | 1.00 | 0.51 | 1SG | 841 |
| ATOM | 841 | CD2 | PHE | 102 | −18.161 | 5.549 | 4.485 | 1.00 | 0.51 | 1SG | 842 |
| ATOM | 842 | CE1 | PHE | 102 | −15.712 | 6.301 | 5.470 | 1.00 | 0.51 | 1SG | 843 |
| ATOM | 843 | CE2 | PHE | 102 | −17.278 | 6.225 | 3.679 | 1.00 | 0.51 | 1SG | 844 |
| ATOM | 844 | CZ | PHE | 102 | −16.052 | 6.602 | 4.173 | 1.00 | 0.51 | 1SG | 845 |
| ATOM | 845 | C | PHE | 102 | −18.459 | 6.081 | 8.514 | 1.00 | 0.51 | 1SG | 846 |
| ATOM | 846 | O | PHE | 102 | −17.972 | 7.202 | 8.389 | 1.00 | 0.51 | 1SG | 847 |
| ATOM | 847 | N | TYR | 103 | −18.151 | 5.195 | 9.496 | 1.00 | 0.53 | 1SG | 848 |
| ATOM | 848 | CA | TYR | 103 | −17.251 | 5.222 | 10.630 | 1.00 | 0.53 | 1SG | 849 |
| ATOM | 849 | CB | TYR | 103 | −15.866 | 4.623 | 10.320 | 1.00 | 0.53 | 1SG | 850 |
| ATOM | 850 | CG | TYR | 103 | −16.093 | 3.232 | 9.830 | 1.00 | 0.53 | 1SG | 851 |
| ATOM | 851 | CD1 | TYR | 103 | −16.310 | 2.178 | 10.690 | 1.00 | 0.53 | 1SG | 852 |
| ATOM | 852 | CD2 | TYR | 103 | −16.088 | 2.989 | 8.479 | 1.00 | 0.53 | 1SG | 853 |
| ATOM | 853 | CE1 | TYR | 103 | −16.519 | 0.907 | 10.203 | 1.00 | 0.53 | 1SG | 854 |
| ATOM | 854 | CE2 | TYR | 103 | −16.296 | 1.723 | 7.984 | 1.00 | 0.53 | 1SG | 855 |
| ATOM | 855 | CZ | TYR | 103 | −16.513 | 0.677 | 8.848 | 1.00 | 0.53 | 1SG | 856 |
| ATOM | 856 | OH | TYR | 103 | −16.728 | −0.624 | 8.344 | 1.00 | 0.53 | 1SG | 857 |
| ATOM | 857 | C | TYR | 103 | −17.058 | 6.567 | 11.297 | 1.00 | 0.53 | 1SG | 858 |
| ATOM | 858 | O | TYR | 103 | −17.052 | 7.627 | 10.676 | 1.00 | 0.53 | 1SG | 859 |
| ATOM | 859 | N | CYS | 104 | −17.145 | 6.551 | 12.644 | 1.00 | 0.50 | 1SG | 860 |
| ATOM | 860 | CA | CYS | 104 | −16.819 | 7.632 | 13.536 | 1.00 | 0.50 | 1SG | 861 |
| ATOM | 861 | CB | CYS | 104 | −17.822 | 7.738 | 14.694 | 1.00 | 0.50 | 1SG | 862 |
| ATOM | 862 | SG | CYS | 104 | −19.503 | 8.092 | 14.099 | 1.00 | 0.50 | 1SG | 863 |
| ATOM | 863 | C | CYS | 104 | −15.470 | 7.451 | 14.138 | 1.00 | 0.50 | 1SG | 864 |
| ATOM | 864 | O | CYS | 104 | −14.842 | 8.406 | 14.595 | 1.00 | 0.50 | 1SG | 865 |
| ATOM | 865 | N | ILE | 105 | −15.044 | 6.179 | 14.255 | 1.00 | 0.45 | 1SG | 866 |
| ATOM | 866 | CA | ILE | 105 | −13.774 | 5.908 | 14.846 | 1.00 | 0.45 | 1SG | 867 |
| ATOM | 867 | CB | ILE | 105 | −13.540 | 4.449 | 15.163 | 1.00 | 0.45 | 1SG | 868 |
| ATOM | 868 | CG2 | ILE | 105 | −14.560 | 4.048 | 16.241 | 1.00 | 0.45 | 1SG | 869 |
| ATOM | 869 | CG1 | ILE | 105 | −13.586 | 3.557 | 13.908 | 1.00 | 0.45 | 1SG | 870 |
| ATOM | 870 | CD1 | ILE | 105 | −14.934 | 3.567 | 13.188 | 1.00 | 0.45 | 1SG | 871 |
| ATOM | 871 | C | ILE | 105 | −12.803 | 6.369 | 13.828 | 1.00 | 0.45 | 1SG | 872 |
| ATOM | 872 | O | ILE | 105 | −12.762 | 5.868 | 12.706 | 1.00 | 0.45 | 1SG | 873 |
| ATOM | 873 | N | HIS | 106 | −12.005 | 7.381 | 14.192 | 1.00 | 0.41 | 1SG | 874 |
| ATOM | 874 | CA | HIS | 106 | −11.206 | 7.949 | 13.162 | 1.00 | 0.41 | 1SG | 875 |
| ATOM | 875 | ND1 | HIS | 106 | −11.648 | 11.288 | 11.241 | 1.00 | 0.41 | 1SG | 876 |
| ATOM | 876 | CG | HIS | 106 | −11.357 | 9.986 | 11.584 | 1.00 | 0.41 | 1SG | 877 |
| ATOM | 877 | CB | HIS | 106 | −11.561 | 9.436 | 12.966 | 1.00 | 0.41 | 1SG | 878 |
| ATOM | 878 | NE2 | HIS | 106 | −10.957 | 10.283 | 9.382 | 1.00 | 0.41 | 1SG | 879 |
| ATOM | 879 | CD2 | HIS | 106 | −10.935 | 9.386 | 10.437 | 1.00 | 0.41 | 1SG | 880 |
| ATOM | 880 | CE1 | HIS | 106 | −11.392 | 11.411 | 9.914 | 1.00 | 0.41 | 1SG | 881 |
| ATOM | 881 | C | HIS | 106 | −9.791 | 7.840 | 13.617 | 1.00 | 0.41 | 1SG | 882 |
| ATOM | 882 | O | HIS | 106 | −9.508 | 7.979 | 14.806 | 1.00 | 0.41 | 1SG | 883 |
| ATOM | 883 | N | VAL | 107 | −8.863 | 7.517 | 12.696 | 1.00 | 0.39 | 1SG | 884 |
| ATOM | 884 | CA | VAL | 107 | −7.500 | 7.538 | 13.116 | 1.00 | 0.39 | 1SG | 885 |
| ATOM | 885 | CB | VAL | 107 | −6.697 | 6.298 | 12.823 | 1.00 | 0.39 | 1SG | 886 |
| ATOM | 886 | CG1 | VAL | 107 | −7.382 | 5.137 | 13.548 | 1.00 | 0.39 | 1SG | 887 |
| ATOM | 887 | CG2 | VAL | 107 | −6.540 | 6.083 | 11.316 | 1.00 | 0.39 | 1SG | 888 |
| ATOM | 888 | C | VAL | 107 | −6.909 | 8.710 | 12.423 | 1.00 | 0.39 | 1SG | 889 |
| ATOM | 889 | O | VAL | 107 | −6.886 | 8.792 | 11.195 | 1.00 | 0.39 | 1SG | 890 |
| ATOM | 890 | N | ASP | 108 | −6.434 | 9.683 | 13.211 | 1.00 | 0.45 | 1SG | 891 |
| ATOM | 891 | CA | ASP | 108 | −5.947 | 10.856 | 12.573 | 1.00 | 0.45 | 1SG | 892 |
| ATOM | 892 | CB | ASP | 108 | −6.210 | 12.150 | 13.366 | 1.00 | 0.45 | 1SG | 893 |
| ATOM | 893 | CG | ASP | 108 | −5.892 | 13.356 | 12.484 | 1.00 | 0.45 | 1SG | 894 |
| ATOM | 894 | OD1 | ASP | 108 | −4.831 | 13.344 | 11.804 | 1.00 | 0.45 | 1SG | 895 |
| ATOM | 895 | OD2 | ASP | 108 | −6.719 | 14.307 | 12.474 | 1.00 | 0.45 | 1SG | 896 |
| ATOM | 896 | C | ASP | 108 | −4.486 | 10.696 | 12.428 | 1.00 | 0.45 | 1SG | 897 |
| ATOM | 897 | O | ASP | 108 | −3.749 | 10.617 | 13.410 | 1.00 | 0.45 | 1SG | 898 |
| ATOM | 898 | N | ARG | 109 | −4.030 | 10.622 | 11.171 | 1.00 | 0.73 | 1SG | 899 |
| ATOM | 899 | CA | ARG | 109 | −2.625 | 10.544 | 11.002 | 1.00 | 0.73 | 1SG | 900 |
| ATOM | 900 | CB | ARG | 109 | −2.201 | 10.097 | 9.596 | 1.00 | 0.73 | 1SG | 901 |
| ATOM | 901 | CG | ARG | 109 | −2.812 | 8.752 | 9.208 | 1.00 | 0.73 | 1SG | 902 |
| ATOM | 902 | CD | ARG | 109 | −2.709 | 7.704 | 10.316 | 1.00 | 0.73 | 1SG | 903 |
| ATOM | 903 | NE | ARG | 109 | −1.301 | 7.234 | 10.374 | 1.00 | 0.73 | 1SG | 904 |
| ATOM | 904 | CZ | ARG | 109 | −0.967 | 6.214 | 11.214 | 1.00 | 0.73 | 1SG | 905 |
| ATOM | 905 | NH1 | ARG | 109 | −1.907 | 5.654 | 12.029 | 1.00 | 0.73 | 1SG | 906 |
| ATOM | 906 | NH2 | ARG | 109 | 0.319 | 5.765 | 11.253 | 1.00 | 0.73 | 1SG | 907 |
| ATOM | 907 | C | ARG | 109 | −2.155 | 11.939 | 11.192 | 1.00 | 0.73 | 1SG | 908 |
| ATOM | 908 | O | ARG | 109 | −1.921 | 12.670 | 10.229 | 1.00 | 0.73 | 1SG | 909 |
| ATOM | 909 | N | LYS | 110 | −2.009 | 12.339 | 12.471 | 1.00 | 0.81 | 1SG | 910 |
| ATOM | 910 | CA | LYS | 110 | −1.458 | 13.619 | 12.787 | 1.00 | 0.81 | 1SG | 911 |
| ATOM | 911 | CB | LYS | 110 | −1.392 | 13.928 | 14.291 | 1.00 | 0.81 | 1SG | 912 |
| ATOM | 912 | CG | LYS | 110 | −1.306 | 15.429 | 14.591 | 1.00 | 0.81 | 1SG | 913 |
| ATOM | 913 | CD | LYS | 110 | −0.134 | 16.139 | 13.909 | 1.00 | 0.81 | 1SG | 914 |
| ATOM | 914 | CE | LYS | 110 | 0.994 | 16.538 | 14.860 | 1.00 | 0.81 | 1SG | 915 |
| ATOM | 915 | NZ | LYS | 110 | 0.617 | 17.762 | 15.601 | 1.00 | 0.81 | 1SG | 916 |
| ATOM | 916 | C | LYS | 110 | −0.076 | 13.456 | 12.286 | 1.00 | 0.81 | 1SG | 917 |
| ATOM | 917 | O | LYS | 110 | 0.589 | 14.415 | 11.900 | 1.00 | 0.81 | 1SG | 918 |
| ATOM | 918 | N | ALA | 111 | 0.379 | 12.187 | 12.353 | 1.00 | 0.55 | 1SG | 919 |
| ATOM | 919 | CA | ALA | 111 | 1.579 | 11.749 | 11.729 | 1.00 | 0.55 | 1SG | 920 |
| ATOM | 920 | CB | ALA | 111 | 1.916 | 10.295 | 12.093 | 1.00 | 0.55 | 1SG | 921 |
| ATOM | 921 | C | ALA | 111 | 1.251 | 11.791 | 10.278 | 1.00 | 0.55 | 1SG | 922 |
| ATOM | 922 | O | ALA | 111 | 1.121 | 10.765 | 9.614 | 1.00 | 0.55 | 1SG | 923 |
| ATOM | 923 | N | GLU | 112 | 1.165 | 13.029 | 9.771 | 1.00 | 0.50 | 1SG | 924 |
| ATOM | 924 | CA | GLU | 112 | 0.719 | 13.402 | 8.472 | 1.00 | 0.50 | 1SG | 925 |
| ATOM | 925 | CB | GLU | 112 | 0.336 | 14.891 | 8.416 | 1.00 | 0.50 | 1SG | 926 |
| ATOM | 926 | CG | GLU | 112 | 1.495 | 15.819 | 8.795 | 1.00 | 0.50 | 1SG | 927 |
| ATOM | 927 | CD | GLU | 112 | 0.969 | 17.238 | 8.980 | 1.00 | 0.50 | 1SG | 928 |
| ATOM | 928 | OE1 | GLU | 112 | 0.045 | 17.637 | 8.225 | 1.00 | 0.50 | 1SG | 929 |
| ATOM | 929 | OE2 | GLU | 112 | 1.484 | 17.942 | 9.890 | 1.00 | 0.50 | 1SG | 930 |
| ATOM | 930 | C | GLU | 112 | 1.789 | 13.167 | 7.468 | 1.00 | 0.50 | 1SG | 931 |
| ATOM | 931 | O | GLU | 112 | 2.560 | 12.213 | 7.553 | 1.00 | 0.50 | 1SG | 932 |
| ATOM | 932 | N | GLU | 113 | 1.808 | 14.060 | 6.465 | 1.00 | 0.55 | 1SG | 933 |
| ATOM | 933 | CA | GLU | 113 | 2.669 | 14.011 | 5.323 | 1.00 | 0.55 | 1SG | 934 |
| ATOM | 934 | CB | GLU | 113 | 2.374 | 15.023 | 4.227 | 1.00 | 0.55 | 1SG | 935 |
| ATOM | 935 | CG | GLU | 113 | 1.061 | 14.729 | 3.548 | 1.00 | 0.55 | 1SG | 936 |
| ATOM | 936 | CD | GLU | 113 | 0.977 | 13.280 | 3.035 | 1.00 | 0.55 | 1SG | 937 |
| ATOM | 937 | OE1 | GLU | 113 | 1.864 | 12.424 | 3.295 | 1.00 | 0.55 | 1SG | 938 |
| ATOM | 938 | OE2 | GLU | 113 | −0.011 | 13.017 | 2.304 | 1.00 | 0.55 | 1SG | 939 |
| ATOM | 939 | C | GLU | 113 | 4.085 | 14.213 | 5.670 | 1.00 | 0.55 | 1SG | 940 |
| ATOM | 940 | O | GLU | 113 | 4.957 | 13.891 | 4.867 | 1.00 | 0.55 | 1SG | 941 |
| ATOM | 941 | N | SER | 114 | 4.353 | 14.731 | 6.872 | 1.00 | 0.40 | 1SG | 942 |
| ATOM | 942 | CA | SER | 114 | 5.671 | 15.130 | 7.261 | 1.00 | 0.40 | 1SG | 943 |
| ATOM | 943 | CB | SER | 114 | 5.772 | 15.523 | 8.742 | 1.00 | 0.40 | 1SG | 944 |
| ATOM | 944 | OG | SER | 114 | 4.977 | 16.670 | 9.003 | 1.00 | 0.40 | 1SG | 945 |
| ATOM | 945 | C | SER | 114 | 6.656 | 14.035 | 7.014 | 1.00 | 0.40 | 1SG | 946 |
| ATOM | 946 | O | SER | 114 | 7.842 | 14.330 | 6.875 | 1.00 | 0.40 | 1SG | 947 |
| ATOM | 947 | N | PHE | 115 | 6.178 | 12.777 | 6.884 | 1.00 | 0.35 | 1SG | 948 |
| ATOM | 948 | CA | PHE | 115 | 6.947 | 11.570 | 6.775 | 1.00 | 0.35 | 1SG | 949 |
| ATOM | 949 | CB | PHE | 115 | 8.421 | 11.735 | 6.316 | 1.00 | 0.35 | 1SG | 950 |
| ATOM | 950 | CG | PHE | 115 | 9.075 | 10.399 | 6.154 | 1.00 | 0.35 | 1SG | 951 |
| ATOM | 951 | CD1 | PHE | 115 | 8.741 | 9.572 | 5.106 | 1.00 | 0.35 | 1SG | 952 |
| ATOM | 952 | CD2 | PHE | 115 | 10.050 | 9.982 | 7.032 | 1.00 | 0.35 | 1SG | 953 |
| ATOM | 953 | CE1 | PHE | 115 | 9.343 | 8.345 | 4.951 | 1.00 | 0.35 | 1SG | 954 |
| ATOM | 954 | CE2 | PHE | 115 | 10.657 | 8.757 | 6.885 | 1.00 | 0.35 | 1SG | 955 |
| ATOM | 955 | CZ | PHE | 115 | 10.301 | 7.934 | 5.844 | 1.00 | 0.35 | 1SG | 956 |
| ATOM | 956 | C | PHE | 115 | 6.899 | 10.992 | 8.132 | 1.00 | 0.35 | 1SG | 957 |
| ATOM | 957 | O | PHE | 115 | 7.816 | 10.313 | 8.589 | 1.00 | 0.35 | 1SG | 958 |
| ATOM | 958 | N | LEU | 116 | 5.782 | 11.298 | 8.822 | 1.00 | 0.37 | 1SG | 959 |
| ATOM | 959 | CA | LEU | 116 | 5.544 | 10.654 | 10.067 | 1.00 | 0.37 | 1SG | 960 |
| ATOM | 960 | CB | LEU | 116 | 4.407 | 11.272 | 10.902 | 1.00 | 0.37 | 1SG | 961 |
| ATOM | 961 | CG | LEU | 116 | 4.493 | 12.792 | 11.148 | 1.00 | 0.37 | 1SG | 962 |
| ATOM | 962 | CD2 | LEU | 116 | 3.957 | 13.206 | 12.532 | 1.00 | 0.37 | 1SG | 963 |
| ATOM | 963 | CD1 | LEU | 116 | 3.923 | 13.586 | 9.973 | 1.00 | 0.37 | 1SG | 964 |
| ATOM | 964 | C | LEU | 116 | 5.089 | 9.301 | 9.644 | 1.00 | 0.37 | 1SG | 965 |
| ATOM | 965 | O | LEU | 116 | 3.901 | 8.976 | 9.683 | 1.00 | 0.37 | 1SG | 966 |
| ATOM | 966 | N | ALA | 117 | 6.063 | 8.482 | 9.211 | 1.00 | 0.22 | 1SG | 967 |
| ATOM | 967 | CA | ALA | 117 | 5.788 | 7.186 | 8.683 | 1.00 | 0.22 | 1SG | 968 |
| ATOM | 968 | CB | ALA | 117 | 6.989 | 6.546 | 7.966 | 1.00 | 0.22 | 1SG | 969 |
| ATOM | 969 | C | ALA | 117 | 5.419 | 6.313 | 9.819 | 1.00 | 0.22 | 1SG | 970 |
| ATOM | 970 | O | ALA | 117 | 6.055 | 6.328 | 10.871 | 1.00 | 0.22 | 1SG | 971 |
| ATOM | 971 | N | ALA | 118 | 4.349 | 5.530 | 9.623 | 1.00 | 0.10 | 1SG | 972 |
| ATOM | 972 | CA | ALA | 118 | 3.926 | 4.645 | 10.652 | 1.00 | 0.10 | 1SG | 973 |
| ATOM | 973 | CB | ALA | 118 | 2.503 | 4.106 | 10.459 | 1.00 | 0.10 | 1SG | 974 |
| ATOM | 974 | C | ALA | 118 | 4.827 | 3.465 | 10.609 | 1.00 | 0.10 | 1SG | 975 |
| ATOM | 975 | O | ALA | 118 | 5.423 | 3.154 | 9.578 | 1.00 | 0.10 | 1SG | 976 |
| ATOM | 976 | N | VAL | 119 | 4.971 | 2.800 | 11.766 | 1.00 | 0.20 | 1SG | 977 |
| ATOM | 977 | CA | VAL | 119 | 5.733 | 1.594 | 11.832 | 1.00 | 0.20 | 1SG | 978 |
| ATOM | 978 | CB | VAL | 119 | 6.080 | 1.225 | 13.253 | 1.00 | 0.20 | 1SG | 979 |
| ATOM | 979 | CG1 | VAL | 119 | 6.908 | −0.072 | 13.273 | 1.00 | 0.20 | 1SG | 980 |
| ATOM | 980 | CG2 | VAL | 119 | 6.798 | 2.425 | 13.897 | 1.00 | 0.20 | 1SG | 981 |
| ATOM | 981 | C | VAL | 119 | 4.826 | 0.557 | 11.245 | 1.00 | 0.20 | 1SG | 982 |
| ATOM | 982 | O | VAL | 119 | 3.668 | 0.851 | 10.954 | 1.00 | 0.20 | 1SG | 983 |
| ATOM | 983 | N | GLN | 120 | 5.323 | −0.668 | 11.018 | 1.00 | 0.30 | 1SG | 984 |
| ATOM | 984 | CA | GLN | 120 | 4.474 | −1.676 | 10.449 | 1.00 | 0.30 | 1SG | 985 |
| ATOM | 985 | CB | GLN | 120 | 5.189 | −2.997 | 10.120 | 1.00 | 0.30 | 1SG | 986 |
| ATOM | 986 | CG | GLN | 120 | 5.730 | −3.766 | 11.323 | 1.00 | 0.30 | 1SG | 987 |
| ATOM | 987 | CD | GLN | 120 | 6.374 | −5.030 | 10.767 | 1.00 | 0.30 | 1SG | 988 |
| ATOM | 988 | OE1 | GLN | 120 | 6.717 | −5.958 | 11.495 | 1.00 | 0.30 | 1SG | 989 |
| ATOM | 989 | NE2 | GLN | 120 | 6.542 | −5.075 | 9.418 | 1.00 | 0.30 | 1SG | 990 |
| ATOM | 990 | C | GLN | 120 | 3.379 | −1.966 | 11.425 | 1.00 | 0.30 | 1SG | 991 |
| ATOM | 991 | O | GLN | 120 | 2.252 | −2.278 | 11.046 | 1.00 | 0.30 | 1SG | 992 |
| ATOM | 992 | N | GLY | 121 | 3.716 | −1.856 | 12.719 | 1.00 | 0.31 | 1SG | 993 |
| ATOM | 993 | CA | GLY | 121 | 2.877 | −2.119 | 13.852 | 1.00 | 0.31 | 1SG | 994 |
| ATOM | 994 | C | GLY | 121 | 1.769 | −1.115 | 13.968 | 1.00 | 0.31 | 1SG | 995 |
| ATOM | 995 | O | GLY | 121 | 0.868 | −1.299 | 14.785 | 1.00 | 0.31 | 1SG | 996 |
| ATOM | 996 | N | ILE | 122 | 1.824 | −0.003 | 13.208 | 1.00 | 0.30 | 1SG | 997 |
| ATOM | 997 | CA | ILE | 122 | 0.878 | 1.061 | 13.410 | 1.00 | 0.30 | 1SG | 998 |
| ATOM | 998 | CB | ILE | 122 | 1.004 | 2.172 | 12.395 | 1.00 | 0.30 | 1SG | 999 |
| ATOM | 999 | CG2 | ILE | 122 | 0.808 | 1.587 | 10.986 | 1.00 | 0.30 | 1SG | 1000 |
| ATOM | 1000 | CD1 | ILE | 122 | 0.031 | 3.319 | 12.718 | 1.00 | 0.30 | 1SG | 1001 |
| ATOM | 1001 | CD1 | ILE | 122 | 0.394 | 4.119 | 13.965 | 1.00 | 0.30 | 1SG | 1002 |
| ATOM | 1002 | C | ILE | 122 | −0.541 | 0.577 | 13.368 | 1.00 | 0.30 | 1SG | 1003 |
| ATOM | 1003 | O | ILE | 122 | −1.292 | 0.842 | 14.303 | 1.00 | 0.30 | 1SG | 1004 |
| ATOM | 1004 | N | ALA | 123 | −0.951 | −0.164 | 12.323 | 1.00 | 0.20 | 1SG | 1005 |
| ATOM | 1005 | CA | ALA | 123 | −2.324 | −0.578 | 12.220 | 1.00 | 0.20 | 1SG | 1006 |
| ATOM | 1006 | CB | ALA | 123 | −2.625 | −1.334 | 10.914 | 1.00 | 0.20 | 1SG | 1007 |
| ATOM | 1007 | C | ALA | 123 | −2.657 | −1.496 | 13.350 | 1.00 | 0.20 | 1SG | 1008 |
| ATOM | 1008 | O | ALA | 123 | −3.709 | −1.374 | 13.976 | 1.00 | 0.20 | 1SG | 1009 |
| ATOM | 1009 | N | SER | 124 | −1.743 | −2.427 | 13.662 | 1.00 | 0.09 | 1SG | 1010 |
| ATOM | 1010 | CA | SER | 124 | −2.020 | −3.408 | 14.668 | 1.00 | 0.09 | 1SG | 1011 |
| ATOM | 1011 | CB | SER | 124 | −0.859 | −4.394 | 14.888 | 1.00 | 0.09 | 1SG | 1012 |
| ATOM | 1012 | OG | SER | 124 | 0.265 | −3.721 | 15.435 | 1.00 | 0.09 | 1SG | 1013 |
| ATOM | 1013 | C | SER | 124 | −2.271 | −2.708 | 15.963 | 1.00 | 0.09 | 1SG | 1014 |
| ATOM | 1014 | O | SER | 124 | −3.147 | −3.101 | 16.731 | 1.00 | 0.09 | 1SG | 1015 |
| ATOM | 1015 | N | CYS | 125 | −1.510 | −1.637 | 16.238 | 1.00 | 0.18 | 1SG | 1016 |
| ATOM | 1016 | CA | CYS | 125 | −1.648 | −0.965 | 17.493 | 1.00 | 0.18 | 1SG | 1017 |
| ATOM | 1017 | CB | CYS | 125 | −0.674 | 0.215 | 17.642 | 1.00 | 0.18 | 1SG | 1018 |
| ATOM | 1018 | SG | CYS | 125 | 1.064 | −0.317 | 17.651 | 1.00 | 0.18 | 1SG | 1019 |
| ATOM | 1019 | C | CYS | 125 | −3.035 | −0.414 | 17.620 | 1.00 | 0.18 | 1SG | 1020 |
| ATOM | 1020 | O | CYS | 125 | −3.662 | −0.530 | 18.671 | 1.00 | 0.18 | 1SG | 1021 |
| ATOM | 1021 | N | PHE | 126 | −3.553 | 0.198 | 16.541 | 1.00 | 0.34 | 1SG | 1022 |
| ATOM | 1022 | CA | PHE | 126 | −4.844 | 0.829 | 16.558 | 1.00 | 0.34 | 1SG | 1023 |
| ATOM | 1023 | CB | PHE | 126 | −5.172 | 1.468 | 15.200 | 1.00 | 0.34 | 1SG | 1024 |
| ATOM | 1024 | CG | PHE | 126 | −4.375 | 2.715 | 15.062 | 1.00 | 0.34 | 1SG | 1025 |
| ATOM | 1025 | CD1 | PHE | 126 | −3.022 | 2.723 | 15.271 | 1.00 | 0.34 | 1SG | 1026 |
| ATOM | 1026 | CD2 | PHE | 126 | −4.976 | 3.880 | 14.674 | 1.00 | 0.34 | 1SG | 1027 |
| ATOM | 1027 | CE1 | PHE | 126 | −2.298 | 3.878 | 15.128 | 1.00 | 0.34 | 1SG | 1028 |
| ATOM | 1028 | CE2 | PHE | 126 | −4.258 | 5.042 | 14.525 | 1.00 | 0.34 | 1SG | 1029 |
| ATOM | 1029 | CZ | PHE | 126 | −2.907 | 5.048 | 14.758 | 1.00 | 0.34 | 1SG | 1030 |
| ATOM | 1030 | C | PHE | 126 | −5.910 | −0.183 | 16.810 | 1.00 | 0.34 | 1SG | 1031 |
| ATOM | 1031 | O | PHE | 126 | −6.773 | 0.009 | 17.664 | 1.00 | 0.34 | 1SG | 1032 |
| ATOM | 1032 | N | ASP | 127 | −5.856 | −1.306 | 16.075 | 1.00 | 0.35 | 1SG | 1033 |
| ATOM | 1033 | CA | ASP | 127 | −6.876 | −2.307 | 16.158 | 1.00 | 0.35 | 1SG | 1034 |
| ATOM | 1034 | CB | ASP | 127 | −6.632 | −3.479 | 15.192 | 1.00 | 0.35 | 1SG | 1035 |
| ATOM | 1035 | CG | ASP | 127 | −6.797 | −2.949 | 13.775 | 1.00 | 0.35 | 1SG | 1036 |
| ATOM | 1036 | OD1 | ASP | 127 | −7.430 | −1.871 | 13.619 | 1.00 | 0.35 | 1SG | 1037 |
| ATOM | 1037 | OD2 | ASP | 127 | −6.287 | −3.611 | 12.832 | 1.00 | 0.35 | 1SG | 1038 |
| ATOM | 1038 | C | ASP | 127 | −6.883 | −2.862 | 17.542 | 1.00 | 0.35 | 1SG | 1039 |
| ATOM | 1039 | O | ASP | 127 | −7.940 | −3.130 | 18.110 | 1.00 | 0.35 | 1SG | 1040 |
| ATOM | 1040 | N | ASN | 128 | −5.690 | −3.050 | 18.126 | 1.00 | 0.26 | 1SG | 1041 |
| ATOM | 1041 | CA | ASN | 128 | −5.616 | −3.653 | 19.420 | 1.00 | 0.26 | 1SG | 1042 |
| ATOM | 1042 | CB | ASN | 128 | −4.168 | −3.858 | 19.892 | 1.00 | 0.26 | 1SG | 1043 |
| ATOM | 1043 | CG | ASN | 128 | −4.207 | −4.563 | 21.240 | 1.00 | 0.26 | 1SG | 1044 |
| ATOM | 1044 | OD1 | ASN | 128 | −4.649 | −5.704 | 21.358 | 1.00 | 0.26 | 1SG | 1045 |
| ATOM | 1045 | ND2 | ASN | 128 | −3.716 | −3.864 | 22.298 | 1.00 | 0.26 | 1SG | 1046 |
| ATOM | 1046 | C | ASN | 128 | −6.296 | −2.802 | 20.457 | 1.00 | 0.26 | 1SG | 1047 |
| ATOM | 1047 | O | ASN | 128 | −7.105 | −3.286 | 21.244 | 1.00 | 0.26 | 1SG | 1048 |
| ATOM | 1048 | N | VAL | 129 | −5.972 | −1.503 | 20.450 | 1.00 | 0.22 | 1SG | 1049 |
| ATOM | 1049 | CA | VAL | 129 | −6.316 | −0.472 | 21.391 | 1.00 | 0.22 | 1SG | 1050 |
| ATOM | 1050 | CB | VAL | 129 | −5.393 | 0.703 | 21.211 | 1.00 | 0.22 | 1SG | 1051 |
| ATOM | 1051 | CG1 | VAL | 129 | −5.700 | 1.843 | 22.195 | 1.00 | 0.22 | 1SG | 1052 |
| ATOM | 1052 | CG2 | VAL | 129 | −3.959 | 0.170 | 21.344 | 1.00 | 0.22 | 1SG | 1053 |
| ATOM | 1053 | C | VAL | 129 | −7.745 | 0.004 | 21.322 | 1.00 | 0.22 | 1SG | 1054 |
| ATOM | 1054 | O | VAL | 129 | −8.175 | 0.713 | 22.229 | 1.00 | 0.22 | 1SG | 1055 |
| ATOM | 1055 | N | PHE | 130 | −8.515 | −0.299 | 20.252 | 1.00 | 0.17 | 1SG | 1056 |
| ATOM | 1056 | CA | PHE | 130 | −9.830 | 0.295 | 20.115 | 1.00 | 0.17 | 1SG | 1057 |
| ATOM | 1057 | CB | PHE | 130 | −10.668 | −0.303 | 18.968 | 1.00 | 0.17 | 1SG | 1058 |
| ATOM | 1058 | CG | PHE | 130 | −10.194 | 0.230 | 17.664 | 1.00 | 0.17 | 1SG | 1059 |
| ATOM | 1059 | CD1 | PHE | 130 | −10.619 | 1.464 | 17.230 | 1.00 | 0.17 | 1SG | 1060 |
| ATOM | 1060 | CD2 | PHE | 130 | −9.346 | −0.503 | 16.868 | 1.00 | 0.17 | 1SG | 1061 |
| ATOM | 1061 | CE1 | PHE | 130 | −10.198 | 1.967 | 16.022 | 1.00 | 0.17 | 1SG | 1062 |
| ATOM | 1062 | CE2 | PHE | 130 | −8.920 | −0.005 | 15.659 | 1.00 | 0.17 | 1SG | 1063 |
| ATOM | 1063 | CZ | PHE | 130 | −9.345 | 1.231 | 15.236 | 1.00 | 0.17 | 1SG | 1064 |
| ATOM | 1064 | C | PHE | 130 | −10.667 | 0.093 | 21.339 | 1.00 | 0.17 | 1SG | 1065 |
| ATOM | 1065 | O | PHE | 130 | −11.167 | 1.062 | 21.909 | 1.00 | 0.17 | 1SG | 1066 |
| ATOM | 1066 | N | VAL | 131 | −10.791 | −1.154 | 21.813 | 1.00 | 0.12 | 1SG | 1067 |
| ATOM | 1067 | CA | VAL | 131 | −11.656 | −1.426 | 22.924 | 1.00 | 0.12 | 1SG | 1068 |
| ATOM | 1068 | CB | VAL | 131 | −11.740 | −2.890 | 23.251 | 1.00 | 0.12 | 1SG | 1069 |
| ATOM | 1069 | CG1 | VAL | 131 | −12.354 | −3.622 | 22.045 | 1.00 | 0.12 | 1SG | 1070 |
| ATOM | 1070 | CG2 | VAL | 131 | −10.341 | −3.396 | 23.641 | 1.00 | 0.12 | 1SG | 1071 |
| ATOM | 1071 | C | VAL | 131 | −11.168 | −0.719 | 24.150 | 1.00 | 0.12 | 1SG | 1072 |
| ATOM | 1072 | O | VAL | 131 | −11.967 | −0.221 | 24.942 | 1.00 | 0.12 | 1SG | 1073 |
| ATOM | 1073 | N | ALA | 132 | −9.839 | −0.658 | 24.345 | 1.00 | 0.09 | 1SG | 1074 |
| ATOM | 1074 | CA | ALA | 132 | −9.289 | −0.065 | 25.531 | 1.00 | 0.09 | 1SG | 1075 |
| ATOM | 1075 | CB | ALA | 132 | −7.751 | −0.143 | 25.574 | 1.00 | 0.09 | 1SG | 1076 |
| ATOM | 1076 | C | ALA | 132 | −9.657 | 1.384 | 25.603 | 1.00 | 0.09 | 1SG | 1077 |
| ATOM | 1077 | O | ALA | 132 | −10.045 | 1.880 | 26.660 | 1.00 | 0.09 | 1SG | 1078 |
| ATOM | 1078 | N | SER | 133 | −9.548 | 2.099 | 24.469 | 1.00 | 0.28 | 1SG | 1079 |
| ATOM | 1079 | CA | SER | 133 | −9.815 | 3.508 | 24.426 | 1.00 | 0.28 | 1SG | 1080 |
| ATOM | 1080 | CB | SER | 133 | −9.587 | 4.095 | 23.020 | 1.00 | 0.28 | 1SG | 1081 |
| ATOM | 1081 | OG | SER | 133 | −9.852 | 5.490 | 23.011 | 1.00 | 0.28 | 1SG | 1082 |
| ATOM | 1082 | C | SER | 133 | −11.249 | 3.727 | 24.776 | 1.00 | 0.28 | 1SG | 1083 |
| ATOM | 1083 | O | SER | 133 | −11.591 | 4.668 | 25.491 | 1.00 | 0.28 | 1SG | 1084 |
| ATOM | 1084 | N | GLN | 134 | −12.132 | 2.853 | 24.269 | 1.00 | 0.59 | 1SG | 1085 |
| ATOM | 1085 | CA | GLN | 134 | −13.532 | 3.016 | 24.498 | 1.00 | 0.59 | 1SG | 1086 |
| ATOM | 1086 | CB | GLN | 134 | −14.367 | 1.909 | 23.828 | 1.00 | 0.59 | 1SG | 1087 |
| ATOM | 1087 | CG | GLN | 134 | −14.165 | 1.798 | 22.315 | 1.00 | 0.59 | 1SG | 1088 |
| ATOM | 1088 | CD | GLN | 134 | −14.774 | 3.019 | 21.646 | 1.00 | 0.59 | 1SG | 1089 |
| ATOM | 1089 | OE1 | GLN | 134 | −14.666 | 3.186 | 20.432 | 1.00 | 0.59 | 1SG | 1090 |
| ATOM | 1090 | NE2 | GLN | 134 | −15.433 | 3.893 | 22.451 | 1.00 | 0.59 | 1SG | 1091 |
| ATOM | 1091 | C | GLN | 134 | −13.807 | 2.916 | 25.967 | 1.00 | 0.59 | 1SG | 1092 |
| ATOM | 1092 | O | GLN | 134 | −14.487 | 3.770 | 26.535 | 1.00 | 0.59 | 1SG | 1093 |
| ATOM | 1093 | N | LEU | 135 | −13.272 | 1.864 | 26.619 | 1.00 | 0.57 | 1SG | 1094 |
| ATOM | 1094 | CA | LEU | 135 | −13.557 | 1.604 | 28.003 | 1.00 | 0.57 | 1SG | 1095 |
| ATOM | 1095 | CB | LEU | 135 | −13.223 | 0.175 | 28.469 | 1.00 | 0.57 | 1SG | 1096 |
| ATOM | 1096 | CG | LEU | 135 | −14.296 | −0.857 | 28.073 | 1.00 | 0.57 | 1SG | 1097 |
| ATOM | 1097 | CD2 | LEU | 135 | −14.113 | −2.176 | 28.841 | 1.00 | 0.57 | 1SG | 1098 |
| ATOM | 1098 | CD1 | LEU | 135 | −14.381 | −1.042 | 26.551 | 1.00 | 0.57 | 1SG | 1099 |
| ATOM | 1099 | C | LEU | 135 | −12.951 | 2.560 | 28.985 | 1.00 | 0.57 | 1SG | 1100 |
| ATOM | 1100 | O | LEU | 135 | −13.619 | 2.936 | 29.947 | 1.00 | 0.57 | 1SG | 1101 |
| ATOM | 1101 | N | GLU | 136 | −11.693 | 3.003 | 28.804 | 1.00 | 0.46 | 1SG | 1102 |
| ATOM | 1102 | CA | GLU | 136 | −11.146 | 3.752 | 29.903 | 1.00 | 0.46 | 1SG | 1103 |
| ATOM | 1103 | CB | GLU | 136 | −9.803 | 3.200 | 30.428 | 1.00 | 0.46 | 1SG | 1104 |
| ATOM | 1104 | CG | GLU | 136 | −8.735 | 2.987 | 29.354 | 1.00 | 0.46 | 1SG | 1105 |
| ATOM | 1105 | CD | GLU | 136 | −7.494 | 2.430 | 30.042 | 1.00 | 0.46 | 1SG | 1106 |
| ATOM | 1106 | OE1 | GLU | 136 | −7.457 | 2.457 | 31.300 | 1.00 | 0.46 | 1SG | 1107 |
| ATOM | 1107 | OE2 | GLU | 136 | −6.569 | 1.971 | 29.323 | 1.00 | 0.46 | 1SG | 1108 |
| ATOM | 1108 | C | GLU | 136 | −11.043 | 5.222 | 29.618 | 1.00 | 0.46 | 1SG | 1109 |
| ATOM | 1109 | O | GLU | 136 | −10.715 | 5.648 | 28.510 | 1.00 | 0.46 | 1SG | 1110 |
| ATOM | 1110 | N | SER | 137 | −11.322 | 6.018 | 30.680 | 1.00 | 0.67 | 1SG | 1111 |
| ATOM | 1111 | CA | SER | 137 | −11.400 | 7.458 | 30.687 | 1.00 | 0.67 | 1SG | 1112 |
| ATOM | 1112 | CB | SER | 137 | −11.970 | 8.018 | 32.004 | 1.00 | 0.67 | 1SG | 1113 |
| ATOM | 1113 | OG | SER | 137 | −13.310 | 7.580 | 32.200 | 1.00 | 0.67 | 1SG | 1114 |
| ATOM | 1114 | C | SER | 137 | −10.049 | 8.073 | 30.560 | 1.00 | 0.67 | 1SG | 1115 |
| ATOM | 1115 | O | SER | 137 | −9.891 | 9.263 | 30.835 | 1.00 | 0.67 | 1SG | 1116 |
| ATOM | 1116 | N | VAL | 138 | −9.059 | 7.268 | 30.140 | 1.00 | 1.00 | 1SG | 1117 |
| ATOM | 1117 | CA | VAL | 138 | −7.713 | 7.670 | 29.878 | 1.00 | 1.00 | 1SG | 1118 |
| ATOM | 1118 | CB | VAL | 138 | −6.839 | 6.410 | 29.844 | 1.00 | 1.00 | 1SG | 1119 |
| ATOM | 1119 | CG1 | VAL | 138 | −7.023 | 5.589 | 28.560 | 1.00 | 1.00 | 1SG | 1120 |
| ATOM | 1120 | CG2 | VAL | 138 | −5.386 | 6.704 | 30.255 | 1.00 | 1.00 | 1SG | 1121 |
| ATOM | 1121 | C | VAL | 138 | −7.880 | 8.466 | 28.598 | 1.00 | 1.00 | 1SG | 1122 |
| ATOM | 1122 | O | VAL | 138 | −8.570 | 9.476 | 28.688 | 1.00 | 1.00 | 1SG | 1123 |
| ATOM | 1123 | N | VAL | 139 | −7.214 | 8.142 | 27.450 | 1.00 | 1.20 | 1SG | 1124 |
| ATOM | 1124 | CA | VAL | 139 | −7.356 | 8.824 | 26.172 | 1.00 | 1.20 | 1SG | 1125 |
| ATOM | 1125 | CB | VAL | 139 | −7.536 | 10.353 | 26.325 | 1.00 | 1.20 | 1SG | 1126 |
| ATOM | 1126 | CG1 | VAL | 139 | −7.033 | 11.288 | 25.229 | 1.00 | 1.20 | 1SG | 1127 |
| ATOM | 1127 | CG2 | VAL | 139 | −9.066 | 10.518 | 26.249 | 1.00 | 1.20 | 1SG | 1128 |
| ATOM | 1128 | C | VAL | 139 | −6.391 | 8.272 | 25.118 | 1.00 | 1.20 | 1SG | 1129 |
| ATOM | 1129 | O | VAL | 139 | −5.727 | 7.263 | 25.356 | 1.00 | 1.20 | 1SG | 1130 |
| ATOM | 1130 | N | TYR | 140 | −6.289 | 8.962 | 23.946 | 1.00 | 1.08 | 1SG | 1131 |
| ATOM | 1131 | CA | TYR | 140 | −5.681 | 8.735 | 22.644 | 1.00 | 1.08 | 1SG | 1132 |
| ATOM | 1132 | CB | TYR | 140 | −5.593 | 9.982 | 21.719 | 1.00 | 1.08 | 1SG | 1133 |
| ATOM | 1133 | CG | TYR | 140 | −4.976 | 11.139 | 22.419 | 1.00 | 1.08 | 1SG | 1134 |
| ATOM | 1134 | CD1 | TYR | 140 | −3.639 | 11.170 | 22.703 | 1.00 | 1.08 | 1SG | 1135 |
| ATOM | 1135 | CD2 | TYR | 140 | −5.742 | 12.229 | 22.750 | 1.00 | 1.08 | 1SG | 1136 |
| ATOM | 1136 | CE1 | TYR | 140 | −3.099 | 12.253 | 23.355 | 1.00 | 1.08 | 1SG | 1137 |
| ATOM | 1137 | CE2 | TYR | 140 | −5.208 | 13.318 | 23.400 | 1.00 | 1.08 | 1SG | 1138 |
| ATOM | 1138 | CZ | TYR | 140 | −3.875 | 13.327 | 23.719 | 1.00 | 1.08 | 1SG | 1139 |
| ATOM | 1139 | OH | TYR | 140 | −3.302 | 14.432 | 24.389 | 1.00 | 1.08 | 1SG | 1140 |
| ATOM | 1140 | C | TYR | 140 | −4.419 | 7.903 | 22.572 | 1.00 | 1.08 | 1SG | 1141 |
| ATOM | 1141 | O | TYR | 140 | −4.317 | 6.905 | 23.280 | 1.00 | 1.08 | 1SG | 1142 |
| ATOM | 1142 | N | ALA | 141 | −3.477 | 8.195 | 21.619 | 1.00 | 0.76 | 1SG | 1143 |
| ATOM | 1143 | CA | ALA | 141 | −2.333 | 7.307 | 21.482 | 1.00 | 0.76 | 1SG | 1144 |
| ATOM | 1144 | CB | ALA | 141 | −2.730 | 5.926 | 20.939 | 1.00 | 0.76 | 1SG | 1145 |
| ATOM | 1145 | C | ALA | 141 | −1.212 | 7.812 | 20.582 | 1.00 | 0.76 | 1SG | 1146 |
| ATOM | 1146 | O | ALA | 141 | −0.996 | 9.007 | 20.426 | 1.00 | 0.76 | 1SG | 1147 |
| ATOM | 1147 | N | SER | 142 | −0.355 | 6.832 | 20.167 | 1.00 | 0.48 | 1SG. | 1148 |
| ATOM | 1148 | CA | SER | 142 | 0.728 | 6.683 | 19.197 | 1.00 | 0.48 | 1SG | 1149 |
| ATOM | 1149 | CB | SER | 142 | 0.312 | 6.800 | 17.722 | 1.00 | 0.48 | 1SG | 1150 |
| ATOM | 1150 | OG | SER | 142 | −0.539 | 5.719 | 17.374 | 1.00 | 0.48 | 1SG | 1151 |
| ATOM | 1151 | C | SER | 142 | 2.015 | 7.461 | 19.371 | 1.00 | 0.48 | 1SG. | 1152 |
| ATOM | 1152 | O | SER | 142 | 3.088 | 6.877 | 19.223 | 1.00 | 0.48 | 1SG | 1153 |
| ATOM | 1153 | N | TRP | 143 | 2.014 | 8.758 | 19.731 | 1.00 | 0.39 | 1SG | 1154 |
| ATOM | 1154 | CA | TRP | 143 | 3.317 | 9.389 | 19.733 | 1.00 | 0.39 | 1SG | 1155 |
| ATOM | 1155 | CB | TRP | 143 | 3.284 | 10.928 | 19.672 | 1.00 | 0.39 | 1SG | 1156 |
| ATOM | 1156 | CG | TRP | 143 | 4.650 | 11.572 | 19.718 | 1.00 | 0.39 | 1SG | 1157 |
| ATOM | 1157 | CD2 | TRP | 143 | 5.592 | 11.545 | 18.635 | 1.00 | 0.39 | 1SG | 1158 |
| ATOM | 1158 | CD1 | TRP | 143 | 5.255 | 12.244 | 20.740 | 1.00 | 0.39 | 1SG | 1159 |
| ATOM | 1159 | NE1 | TRP | 143 | 6.509 | 12.651 | 20.356 | 1.00 | 0.39 | 1SG | 1160 |
| ATOM | 1160 | CE2 | TRP | 143 | 6.732 | 12.224 | 19.063 | 1.00 | 0.39 | 1SG | 1161 |
| ATOM | 1161 | CE3 | TRP | 143 | 5.513 | 10.996 | 17.387 | 1.00 | 0.39 | 1SG | 1162 |
| ATOM | 1162 | CZ2 | TRP | 143 | 7.817 | 12.365 | 18.245 | 1.00 | 0.39 | 1SG | 1163 |
| ATOM | 1163 | CZ3 | TRP | 143 | 6.607 | 11.144 | 16.563 | 1.00 | 0.39 | 1SG | 1164 |
| ATOM | 1164 | CH2 | TRP | 143 | 7.737 | 11.816 | 16.984 | 1.00 | 0.39 | 1SG | 1165 |
| ATOM | 1165 | C | TRP | 143 | 4.083 | 8.973 | 20.952 | 1.00 | 0.39 | 1SG | 1166 |
| ATOM | 1166 | O | TRP | 143 | 3.482 | 8.720 | 21.995 | 1.00 | 0.39 | 1SG | 1167 |
| ATOM | 1167 | N | THR | 144 | 5.436 | 8.865 | 20.845 | 1.00 | 0.58 | 1SG | 1168 |
| ATOM | 1168 | CA | THR | 144 | 6.199 | 8.487 | 22.010 | 1.00 | 0.58 | 1SG | 1169 |
| ATOM | 1169 | CB | THR | 144 | 6.874 | 7.134 | 22.013 | 1.00 | 0.58 | 1SG | 1170 |
| ATOM | 1170 | OG1 | THR | 144 | 7.115 | 6.756 | 23.358 | 1.00 | 0.58 | 1SG | 1171 |
| ATOM | 1171 | CG2 | THR | 144 | 8.264 | 7.220 | 21.363 | 1.00 | 0.58 | 1SG | 1172 |
| ATOM | 1172 | C | THR | 144 | 7.301 | 9.466 | 22.273 | 1.00 | 0.58 | 1SG | 1173 |
| ATOM | 1173 | O | THR | 144 | 8.010 | 9.932 | 21.380 | 1.00 | 0.58 | 1SG | 1174 |
| ATOM | 1174 | N | ARG | 145 | 7.402 | 9.841 | 23.555 | 1.00 | 0.74 | 1SG | 1175 |
| ATOM | 1175 | CA | ARG | 145 | 8.409 | 10.682 | 24.122 | 1.00 | 0.74 | 1SG | 1176 |
| ATOM | 1176 | CB | ARG | 145 | 7.949 | 11.194 | 25.492 | 1.00 | 0.74 | 1SG | 1177 |
| ATOM | 1177 | CG | ARG | 145 | 9.027 | 11.847 | 26.351 | 1.00 | 0.74 | 1SG | 1178 |
| ATOM | 1178 | CD | ARG | 145 | 9.593 | 10.877 | 27.390 | 1.00 | 0.74 | 1SG | 1179 |
| ATOM | 1179 | NE | ARG | 145 | 10.112 | 11.697 | 28.516 | 1.00 | 0.74 | 1SG | 1180 |
| ATOM | 1180 | CZ | ARG | 145 | 9.674 | 11.432 | 29.781 | 1.00 | 0.74 | 1SG | 1181 |
| ATOM | 1181 | NH1 | ARG | 145 | 8.888 | 10.342 | 30.017 | 1.00 | 0.74 | 1SG | 1182 |
| ATOM | 1182 | NH2 | ARG | 145 | 9.993 | 12.269 | 30.809 | 1.00 | 0.74 | 1SG | 1183 |
| ATOM | 1183 | C | ARG | 145 | 9.686 | 9.920 | 24.303 | 1.00 | 0.74 | 1SG | 1184 |
| ATOM | 1184 | O | ARG | 145 | 10.777 | 10.469 | 24.147 | 1.00 | 0.74 | 1SG | 1185 |
| ATOM | 1185 | N | VAL | 146 | 9.585 | 8.616 | 24.626 | 1.00 | 0.69 | 1SG | 1186 |
| ATOM | 1186 | CA | VAL | 146 | 10.766 | 7.904 | 25.020 | 1.00 | 0.69 | 1SG | 1187 |
| ATOM | 1187 | CB | VAL | 146 | 10.555 | 6.551 | 25.626 | 1.00 | 0.69 | 1SG | 1188 |
| ATOM | 1188 | CG1 | VAL | 146 | 9.646 | 6.685 | 26.852 | 1.00 | 0.69 | 1SG | 1189 |
| ATOM | 1189 | CG2 | VAL | 146 | 10.084 | 5.580 | 24.545 | 1.00 | 0.69 | 1SG | 1190 |
| ATOM | 1190 | C | VAL | 146 | 11.699 | 7.693 | 23.886 | 1.00 | 0.69 | 1SG | 1191 |
| ATOM | 1191 | O | VAL | 146 | 11.346 | 7.795 | 22.712 | 1.00 | 0.69 | 1SG | 1192 |
| ATOM | 1192 | N | LYS | 147 | 12.957 | 7.415 | 24.272 | 1.00 | 0.52 | 1SG | 1193 |
| ATOM | 1193 | CA | LYS | 147 | 14.025 | 7.119 | 23.377 | 1.00 | 0.52 | 1SG | 1194 |
| ATOM | 1194 | CB | LYS | 147 | 15.365 | 7.737 | 23.821 | 1.00 | 0.52 | 1SG | 1195 |
| ATOM | 1195 | CG | LYS | 147 | 15.761 | 7.431 | 25.265 | 1.00 | 0.52 | 1SG | 1196 |
| ATOM | 1196 | CD | LYS | 147 | 17.115 | 8.033 | 25.654 | 1.00 | 0.52 | 1SG | 1197 |
| ATOM | 1197 | CE | LYS | 147 | 17.084 | 9.559 | 25.787 | 1.00 | 0.52 | 1SG | 1198 |
| ATOM | 1198 | NZ | LYS | 147 | 18.423 | 10.065 | 26.164 | 1.00 | 0.52 | 1SG | 1199 |
| ATOM | 1199 | C | LYS | 147 | 14.130 | 5.629 | 23.303 | 1.00 | 0.52 | 1SG | 1200 |
| ATOM | 1200 | O | LYS | 147 | 13.389 | 4.908 | 23.969 | 1.00 | 0.52 | 1SG | 1201 |
| ATOM | 1201 | N | ALA | 148 | 15.053 | 5.135 | 22.460 | 1.00 | 0.27 | 1SG | 1202 |
| ATOM | 1202 | CA | ALA | 148 | 15.222 | 3.727 | 22.238 | 1.00 | 0.27 | 1SG | 1203 |
| ATOM | 1203 | CB | ALA | 148 | 16.252 | 3.421 | 21.135 | 1.00 | 0.27 | 1SG | 1204 |
| ATOM | 1204 | C | ALA | 148 | 15.695 | 3.064 | 23.491 | 1.00 | 0.27 | 1SG | 1205 |
| ATOM | 1205 | O | ALA | 148 | 15.312 | 1.936 | 23.795 | 1.00 | 0.27 | 1SG | 1206 |
| ATOM | 1206 | N | ASP | 149 | 16.537 | 3.765 | 24.260 | 1.00 | 0.18 | 1SG | 1207 |
| ATOM | 1207 | CA | ASP | 149 | 17.172 | 3.186 | 25.404 | 1.00 | 0.18 | 1SG | 1208 |
| ATOM | 1208 | CB | ASP | 149 | 18.136 | 4.167 | 26.085 | 1.00 | 0.18 | 1SG | 1209 |
| ATOM | 1209 | CG | ASP | 149 | 19.294 | 4.407 | 25.129 | 1.00 | 0.18 | 1SG | 1210 |
| ATOM | 1210 | OD1 | ASP | 149 | 19.873 | 3.405 | 24.629 | 1.00 | 0.18 | 1SG | 1211 |
| ATOM | 1211 | OD2 | ASP | 149 | 19.616 | 5.600 | 24.885 | 1.00 | 0.18 | 1SG | 1212 |
| ATOM | 1212 | C | ASP | 149 | 16.175 | 2.749 | 26.434 | 1.00 | 0.18 | 1SG | 1213 |
| ATOM | 1213 | O | ASP | 149 | 16.341 | 1.699 | 27.051 | 1.00 | 0.18 | 1SG | 1214 |
| ATOM | 1214 | N | LEU | 150 | 15.102 | 3.525 | 26.650 | 1.00 | 0.24 | 1SG | 1215 |
| ATOM | 1215 | CA | LEU | 150 | 14.244 | 3.238 | 27.766 | 1.00 | 0.24 | 1SG | 1216 |
| ATOM | 1216 | CB | LEU | 150 | 13.076 | 4.229 | 27.807 | 1.00 | 0.24 | 1SG | 1217 |
| ATOM | 1217 | CG | LEU | 150 | 13.576 | 5.682 | 27.871 | 1.00 | 0.24 | 1SG | 1218 |
| ATOM | 1218 | CD2 | LEU | 150 | 14.579 | 5.881 | 29.017 | 1.00 | 0.24 | 1SG | 1219 |
| ATOM | 1219 | CD1 | LEU | 150 | 12.413 | 6.674 | 27.918 | 1.00 | 0.24 | 1SG | 1220 |
| ATOM | 1220 | C | LEU | 150 | 13.679 | 1.847 | 27.722 | 1.00 | 0.24 | 1SG | 1221 |
| ATOM | 1221 | O | LEU | 150 | 14.000 | 1.040 | 28.593 | 1.00 | 0.24 | 1SG | 1222 |
| ATOM | 1222 | N | ASN | 151 | 12.849 | 1.493 | 26.718 | 1.00 | 0.25 | 1SG | 1223 |
| ATOM | 1223 | CA | ASN | 151 | 12.346 | 0.144 | 26.748 | 1.00 | 0.25 | 1SG | 1224 |
| ATOM | 1224 | CB | ASN | 151 | 11.448 | −0.142 | 27.964 | 1.00 | 0.25 | 1SG | 1225 |
| ATOM | 1225 | CG | ASN | 151 | 11.046 | −1.609 | 27.926 | 1.00 | 0.25 | 1SG | 1226 |
| ATOM | 1226 | OD1 | ASN | 151 | 11.709 | −2.464 | 28.510 | 1.00 | 0.25 | 1SG | 1227 |
| ATOM | 1227 | ND2 | ASN | 151 | 9.922 | −1.910 | 27.221 | 1.00 | 0.25 | 1SG | 1228 |
| ATOM | 1228 | C | ASN | 151 | 11.522 | −0.123 | 25.524 | 1.00 | 0.25 | 1SG | 1229 |
| ATOM | 1229 | O | ASN | 151 | 10.428 | 0.416 | 25.364 | 1.00 | 0.25 | 1SG | 1230 |
| ATOM | 1230 | N | CYS | 152 | 12.050 | −0.973 | 24.621 | 1.00 | 0.28 | 1SG | 1231 |
| ATOM | 1231 | CA | CYS | 152 | 11.385 | −1.358 | 23.408 | 1.00 | 0.28 | 1SG | 1232 |
| ATOM | 1232 | CB | CYS | 152 | 12.351 | −1.944 | 22.365 | 1.00 | 0.28 | 1SG | 1233 |
| ATOM | 1233 | SG | CYS | 152 | 13.550 | −0.713 | 21.769 | 1.00 | 0.28 | 1SG | 1234 |
| ATOM | 1234 | C | CYS | 152 | 10.311 | −2.379 | 23.655 | 1.00 | 0.28 | 1SG | 1235 |
| ATOM | 1235 | O | CYS | 152 | 9.278 | −2.380 | 22.987 | 1.00 | 0.28 | 1SG | 1236 |
| ATOM | 1236 | N | MET | 153 | 10.520 | −3.267 | 24.645 | 1.00 | 0.33 | 1SG | 1237 |
| ATOM | 1237 | CA | MET | 153 | 9.693 | −4.427 | 24.839 | 1.00 | 0.33 | 1SG | 1238 |
| ATOM | 1238 | CB | MET | 153 | 10.054 | −5.180 | 26.125 | 1.00 | 0.33 | 1SG | 1239 |
| ATOM | 1239 | CG | MET | 153 | 11.360 | −5.949 | 26.023 | 1.00 | 0.33 | 1SG | 1240 |
| ATOM | 1240 | SD | MET | 153 | 11.202 | −7.481 | 25.062 | 1.00 | 0.33 | 1SG | 1241 |
| ATOM | 1241 | CE | MET | 153 | 10.949 | −6.696 | 23.445 | 1.00 | 0.33 | 1SG | 1242 |
| ATOM | 1242 | C | MET | 153 | 8.246 | −4.106 | 24.976 | 1.00 | 0.33 | 1SG | 1243 |
| ATOM | 1243 | O | MET | 153 | 7.419 | −4.648 | 24.244 | 1.00 | 0.33 | 1SG | 1244 |
| ATOM | 1244 | N | LYS | 154 | 7.889 | −3.213 | 25.908 | 1.00 | 0.34 | 1SG | 1245 |
| ATOM | 1245 | CA | LYS | 154 | 6.491 | −3.002 | 26.119 | 1.00 | 0.34 | 1SG | 1246 |
| ATOM | 1246 | CB | LYS | 154 | 6.116 | −2.422 | 27.493 | 1.00 | 0.34 | 1SG | 1247 |
| ATOM | 1247 | CG | LYS | 154 | 6.234 | −3.384 | 28.674 | 1.00 | 0.34 | 1SG | 1248 |
| ATOM | 1248 | CD | LYS | 154 | 5.995 | −2.666 | 30.004 | 1.00 | 0.34 | 1SG | 1249 |
| ATOM | 1249 | CE | LYS | 154 | 5.838 | −3.600 | 31.200 | 1.00 | 0.34 | 1SG | 1250 |
| ATOM | 1250 | NZ | LYS | 154 | 5.569 | −2.805 | 32.419 | 1.00 | 0.34 | 1SG | 1251 |
| ATOM | 1251 | C | LYS | 154 | 5.968 | −2.012 | 25.140 | 1.00 | 0.34 | 1SG | 1252 |
| ATOM | 1252 | O | LYS | 154 | 6.709 | −1.326 | 24.437 | 1.00 | 0.34 | 1SG | 1253 |
| ATOM | 1253 | N | ASP | 155 | 4.625 | −1.952 | 25.090 | 1.00 | 0.61 | 1SG | 1254 |
| ATOM | 1254 | CA | ASP | 155 | 3.863 | −1.051 | 24.288 | 1.00 | 0.61 | 1SG | 1255 |
| ATOM | 1255 | CB | ASP | 155 | 2.898 | −1.813 | 23.353 | 1.00 | 0.61 | 1SG | 1256 |
| ATOM | 1256 | CG | ASP | 155 | 2.369 | −0.914 | 22.242 | 1.00 | 0.61 | 1SG | 1257 |
| ATOM | 1257 | CG1 | ASP | 155 | 3.166 | −0.119 | 21.684 | 1.00 | 0.61 | 1SG | 1258 |
| ATOM | 1258 | OD2 | ASP | 155 | 1.155 | −1.011 | 21.929 | 1.00 | 0.61 | 1SG | 1259 |
| ATOM | 1259 | C | ASP | 155 | 3.038 | −0.342 | 25.309 | 1.00 | 0.61 | 1SG | 1260 |
| ATOM | 1260 | O | ASP | 155 | 2.712 | −0.922 | 26.344 | 1.00 | 0.61 | 1SG | 1261 |
| ATOM | 1261 | N | LEU | 156 | 2.658 | 0.922 | 25.072 | 1.00 | 1.07 | 1SG | 1262 |
| ATOM | 1262 | CA | LEU | 156 | 1.997 | 1.562 | 26.155 | 1.00 | 1.07 | 1SG | 1263 |
| ATOM | 1263 | CB | LEU | 156 | 2.970 | 2.485 | 26.888 | 1.00 | 1.07 | 1SG | 1264 |
| ATOM | 1264 | CG | LEU | 156 | 2.584 | 2.704 | 28.349 | 1.00 | 1.07 | 1SG | 1265 |
| ATOM | 1265 | CD2 | LEU | 156 | 3.661 | 3.531 | 29.046 | 1.00 | 1.07 | 1SG | 1266 |
| ATOM | 1266 | CD1 | LEU | 156 | 2.394 | 1.369 | 29.082 | 1.00 | 1.07 | 1SG | 1267 |
| ATOM | 1267 | C | LEU | 156 | 0.782 | 2.285 | 25.627 | 1.00 | 1.07 | 1SG | 1268 |
| ATOM | 1268 | O | LEU | 156 | 0.049 | 1.727 | 24.814 | 1.00 | 1.07 | 1SG | 1269 |
| ATOM | 1269 | N | TYR | 157 | 0.551 | 3.544 | 26.075 | 1.00 | 1.33 | 1SG | 1270 |
| ATOM | 1270 | CA | TYR | 157 | −0.670 | 4.284 | 25.835 | 1.00 | 1.33 | 1SG | 1271 |
| ATOM | 1271 | CB | TYR | 157 | −1.233 | 4.915 | 27.127 | 1.00 | 1.33 | 1SG | 1272 |
| ATOM | 1272 | CG | TYR | 157 | −1.554 | 3.803 | 28.071 | 1.00 | 1.33 | 1SG | 1273 |
| ATOM | 1273 | CD1 | TYR | 157 | −2.790 | 3.193 | 28.083 | 1.00 | 1.33 | 1SG | 1274 |
| ATOM | 1274 | CD2 | TYR | 157 | −0.604 | 3.352 | 28.961 | 1.00 | 1.33 | 1SG | 1275 |
| ATOM | 1275 | CE1 | TYR | 157 | −3.072 | 2.172 | 28.959 | 1.00 | 1.33 | 1SG | 1276 |
| ATOM | 1276 | CE2 | TYR | 157 | −0.875 | 2.329 | 29.839 | 1.00 | 1.33 | 1SG | 1277 |
| ATOM | 1277 | CZ | TYR | 157 | −2.114 | 1.736 | 29.839 | 1.00 | 1.33 | 1SG | 1278 |
| ATOM | 1278 | OH | TYR | 157 | −2.414 | 0.685 | 30.732 | 1.00 | 1.33 | 1SG | 1279 |
| ATOM | 1279 | C | TYR | 157 | −0.577 | 5.372 | 24.776 | 1.00 | 1.33 | 1SG | 1280 |
| ATOM | 1280 | O | TYR | 157 | −0.030 | 5.192 | 23.693 | 1.00 | 1.33 | 1SG | 1281 |
| ATOM | 1281 | N | ARG | 158 | −1.208 | 6.529 | 25.071 | 1.00 | 1.39 | 1SG | 1282 |
| ATOM | 1282 | CA | ARG | 158 | −1.477 | 7.705 | 24.264 | 1.00 | 1.39 | 1SG | 1283 |
| ATOM | 1283 | CB | ARG | 158 | −2.714 | 8.360 | 24.911 | 1.00 | 1.39 | 1SG | 1284 |
| ATOM | 1284 | CG | ARG | 158 | −2.848 | 9.842 | 25.170 | 1.00 | 1.39 | 1SG | 1285 |
| ATOM | 1285 | CD | ARG | 158 | −4.276 | 10.253 | 25.466 | 1.00 | 1.39 | 1SG | 1286 |
| ATOM | 1286 | NE | ARG | 158 | −4.526 | 10.225 | 26.933 | 1.00 | 1.39 | 1SG | 1287 |
| ATOM | 1287 | CZ | ARG | 158 | −4.576 | 9.100 | 27.710 | 1.00 | 1.39 | 1SG | 1288 |
| ATOM | 1288 | NH1 | ARG | 158 | −4.088 | 7.884 | 27.342 | 1.00 | 1.39 | 1SG | 1289 |
| ATOM | 1289 | NH2 | ARG | 158 | −5.174 | 9.197 | 28.925 | 1.00 | 1.39 | 1SG | 1290 |
| ATOM | 1290 | C | ARG | 158 | −0.299 | 8.641 | 24.043 | 1.00 | 1.39 | 1SG | 1291 |
| ATOM | 1291 | O | ARG | 158 | 0.854 | 8.229 | 24.117 | 1.00 | 1.39 | 1SG | 1292 |
| ATOM | 1292 | N | MET | 159 | −0.584 | 9.894 | 23.592 | 1.00 | 1.27 | 1SG | 1293 |
| ATOM | 1293 | CA | MET | 159 | 0.362 | 10.973 | 23.489 | 1.00 | 1.27 | 1SG | 1294 |
| ATOM | 1294 | CB | MET | 159 | 1.794 | 10.579 | 23.090 | 1.00 | 1.27 | 1SG | 1295 |
| ATOM | 1295 | CG | MET | 159 | 2.746 | 11.779 | 22.999 | 1.00 | 1.27 | 1SG | 1296 |
| ATOM | 1296 | SD | MET | 159 | 3.057 | 12.657 | 24.564 | 1.00 | 1.27 | 1SG | 1297 |
| ATOM | 1297 | CE | MET | 159 | 3.961 | 11.311 | 25.379 | 1.00 | 1.27 | 1SG | 1298 |
| ATOM | 1298 | C | MET | 159 | −0.118 | 11.989 | 22.506 | 1.00 | 1.27 | 1SG | 1299 |
| ATOM | 1299 | O | MET | 159 | −0.543 | 13.073 | 22.897 | 1.00 | 1.27 | 1SG | 1300 |
| ATOM | 1300 | N | ASN | 160 | −0.014 | 11.681 | 21.198 | 1.00 | 0.97 | 1SG | 1301 |
| ATOM | 1301 | CA | ASN | 160 | −0.430 | 12.564 | 20.141 | 1.00 | 0.97 | 1SG | 1302 |
| ATOM | 1302 | CB | ASN | 160 | 0.231 | 13.961 | 20.098 | 1.00 | 0.97 | 1SG | 1303 |
| ATOM | 1303 | CG | ASN | 160 | −0.557 | 14.956 | 20.937 | 1.00 | 0.97 | 1SG | 1304 |
| ATOM | 1304 | OD1 | ASN | 160 | −1.729 | 14.738 | 21.242 | 1.00 | 0.97 | 1SG | 1305 |
| ATOM | 1305 | ND2 | ASN | 160 | 0.104 | 16.080 | 21.326 | 1.00 | 0.97 | 1SG | 1306 |
| ATOM | 1306 | C | ASN | 160 | −0.047 | 11.900 | 18.867 | 1.00 | 0.97 | 1SG | 1307 |
| ATOM | 1307 | O | ASN | 160 | −0.216 | 10.693 | 18.717 | 1.00 | 0.97 | 1SG | 1308 |
| ATOM | 1308 | N | ALA | 161 | 0.481 | 12.696 | 17.914 | 1.00 | 0.59 | 1SG | 1309 |
| ATOM | 1309 | CA | ALA | 161 | 0.894 | 12.188 | 16.638 | 1.00 | 0.59 | 1SG | 1310 |
| ATOM | 1310 | CB | ALA | 161 | 2.161 | 11.316 | 16.722 | 1.00 | 0.59 | 1SG | 1311 |
| ATOM | 1311 | C | ALA | 161 | −0.217 | 11.365 | 16.083 | 1.00 | 0.59 | 1SG | 1312 |
| ATOM | 1312 | O | ALA | 161 | −1.362 | 11.810 | 16.034 | 1.00 | 0.59 | 1SG | 1313 |
| ATOM | 1313 | N | ASN | 162 | 0.098 | 10.134 | 15.647 | 1.00 | 0.33 | 1SG | 1314 |
| ATOM | 1314 | CA | ASN | 162 | −0.937 | 9.289 | 15.132 | 1.00 | 0.33 | 1SG | 1315 |
| ATOM | 1315 | CB | ASN | 162 | −0.435 | 7.873 | 14.839 | 1.00 | 0.33 | 1SG | 1316 |
| ATOM | 1316 | CG | ASN | 162 | 0.705 | 8.013 | 13.856 | 1.00 | 0.33 | 1SG | 1317 |
| ATOM | 1317 | OD1 | ASN | 162 | 0.495 | 8.024 | 12.648 | 1.00 | 0.33 | 1SG | 1318 |
| ATOM | 1318 | ND2 | ASN | 162 | 1.946 | 8.160 | 14.382 | 1.00 | 0.33 | 1SG | 1319 |
| ATOM | 1319 | C | ASN | 162 | −1.926 | 9.139 | 16.243 | 1.00 | 0.33 | 1SG | 1320 |
| ATOM | 1320 | O | ASN | 162 | −1.562 | 8.732 | 17.342 | 1.00 | 0.33 | 1SG | 1321 |
| ATOM | 1321 | N | TRP | 163 | −3.209 | 9.481 | 16.035 | 1.00 | 0.39 | 1SG | 1322 |
| ATOM | 1322 | CA | TRP | 163 | −4.057 | 9.265 | 17.169 | 1.00 | 0.39 | 1SG | 1323 |
| ATOM | 1323 | CB | TRP | 163 | −4.046 | 10.447 | 18.162 | 1.00 | 0.39 | 1SG | 1324 |
| ATOM | 1324 | CG | TRP | 163 | −4.534 | 11.768 | 17.616 | 1.00 | 0.39 | 1SG | 1325 |
| ATOM | 1325 | CD2 | TRP | 163 | −5.794 | 12.364 | 17.955 | 1.00 | 0.39 | 1SG | 1326 |
| ATOM | 1326 | CD1 | TRP | 163 | −3.899 | 12.654 | 16.794 | 1.00 | 0.39 | 1SG | 1327 |
| ATOM | 1327 | NE1 | TRP | 163 | −4.693 | 13.757 | 16.586 | 1.00 | 0.39 | 1SG | 1328 |
| ATOM | 1328 | CE2 | TRP | 163 | −5.861 | 13.594 | 17.300 | 1.00 | 0.39 | 1SG | 1329 |
| ATOM | 1329 | CE3 | TRP | 163 | −6.807 | 11.927 | 18.757 | 1.00 | 0.39 | 1SG | 1330 |
| ATOM | 1330 | CZ2 | TRP | 163 | −6.950 | 14.407 | 17.434 | 1.00 | 0.39 | 1SG | 1331 |
| ATOM | 1331 | CZ3 | TRP | 163 | −7.906 | 12.745 | 18.883 | 1.00 | 0.39 | 1SG | 1332 |
| ATOM | 1332 | CH2 | TRP | 163 | −7.978 | 13.960 | 18.234 | 1.00 | 0.39 | 1SG | 1333 |
| ATOM | 1333 | C | TRP | 163 | −5.451 | 8.995 | 16.710 | 1.00 | 0.39 | 1SG | 1334 |
| ATOM | 1334 | O | TRP | 163 | −5.971 | 9.673 | 15.827 | 1.00 | 0.39 | 1SG | 1335 |
| ATOM | 1335 | N | LYS | 164 | −6.092 | 7.984 | 17.332 | 1.00 | 0.58 | 1SG | 1336 |
| ATOM | 1336 | CA | LYS | 164 | −7.429 | 7.585 | 17.002 | 1.00 | 0.58 | 1SG | 1337 |
| ATOM | 1337 | CB | LYS | 164 | −7.686 | 6.097 | 17.228 | 1.00 | 0.58 | 1SG | 1338 |
| ATOM | 1338 | CG | LYS | 164 | −6.554 | 5.175 | 16.803 | 1.00 | 0.58 | 1SG | 1339 |
| ATOM | 1339 | CD | LYS | 164 | −6.617 | 3.840 | 17.545 | 1.00 | 0.58 | 1SG | 1340 |
| ATOM | 1340 | CE | LYS | 164 | −6.296 | 3.960 | 19.043 | 1.00 | 0.58 | 1SG | 1341 |
| ATOM | 1341 | NZ | LYS | 164 | −7.351 | 4.717 | 19.761 | 1.00 | 0.58 | 1SG | 1342 |
| ATOM | 1342 | C | LYS | 164 | −8.290 | 8.206 | 18.044 | 1.00 | 0.58 | 1SG | 1343 |
| ATOM | 1343 | O | LYS | 164 | −7.937 | 8.217 | 19.222 | 1.00 | 0.58 | 1SG | 1344 |
| ATOM | 1344 | N | TYR | 165 | −9.453 | 8.741 | 17.645 | 1.00 | 0.58 | 1SG | 1345 |
| ATOM | 1345 | CA | TYR | 165 | −10.301 | 9.323 | 18.634 | 1.00 | 0.58 | 1SG | 1346 |
| ATOM | 1346 | CB | TYR | 165 | −10.273 | 10.861 | 18.612 | 1.00 | 0.58 | 1SG | 1347 |
| ATOM | 1347 | CG | TYR | 165 | −10.784 | 11.347 | 19.925 | 1.00 | 0.58 | 1SG | 1348 |
| ATOM | 1348 | CD1 | TYR | 165 | −9.960 | 11.298 | 21.027 | 1.00 | 0.58 | 1SG | 1349 |
| ATOM | 1349 | CD2 | TYR | 165 | −12.054 | 11.858 | 20.068 | 1.00 | 0.58 | 1SG | 1350 |
| ATOM | 1350 | CE1 | TYR | 165 | −10.390 | 11.740 | 22.255 | 1.00 | 0.58 | 1SG | 1351 |
| ATOM | 1351 | CE2 | TYR | 165 | −12.492 | 12.303 | 21.295 | 1.00 | 0.58 | 1SG | 1352 |
| ATOM | 1352 | CZ | TYR | 165 | −11.660 | 12.244 | 22.389 | 1.00 | 0.58 | 1SG | 1353 |
| ATOM | 1353 | OH | TYR | 165 | −12.104 | 12.700 | 23.648 | 1.00 | 0.58 | 1SG | 1354 |
| ATOM | 1354 | C | TYR | 165 | −11.678 | 8.874 | 18.273 | 1.00 | 0.58 | 1SG | 1355 |
| ATOM | 1355 | O | TYR | 165 | −11.924 | 8.414 | 17.159 | 1.00 | 0.58 | 1SG | 1356 |
| ATOM | 1356 | N | LEU | 166 | −12.620 | 8.993 | 19.222 | 1.00 | 0.54 | 1SG | 1357 |
| ATOM | 1357 | CA | LEU | 166 | −13.959 | 8.543 | 18.994 | 1.00 | 0.54 | 1SG | 1358 |
| ATOM | 1358 | CB | LEU | 166 | −14.784 | 8.402 | 20.284 | 1.00 | 0.54 | 1SG | 1359 |
| ATOM | 1359 | CG | LEU | 166 | −14.257 | 7.315 | 21.239 | 1.00 | 0.54 | 1SG | 1360 |
| ATOM | 1360 | CD2 | LEU | 166 | −12.829 | 7.633 | 21.709 | 1.00 | 0.54 | 1SG | 1361 |
| ATOM | 1361 | CD1 | LEU | 166 | −14.386 | 5.914 | 20.622 | 1.00 | 0.54 | 1SG | 1362 |
| ATOM | 1362 | C | LEU | 166 | −14.661 | 9.531 | 18.120 | 1.00 | 0.54 | 1SG | 1363 |
| ATOM | 1363 | O | LEU | 166 | −14.071 | 10.167 | 17.248 | 1.00 | 0.54 | 1SG | 1364 |
| ATOM | 1364 | N | ILE | 167 | −15.977 | 9.649 | 18.350 | 1.00 | 0.56 | 1SG | 1365 |
| ATOM | 1365 | CA | ILE | 167 | −16.878 | 10.472 | 17.605 | 1.00 | 0.56 | 1SG | 1366 |
| ATOM | 1366 | CB | ILE | 167 | −18.300 | 10.343 | 18.055 | 1.00 | 0.56 | 1SG | 1367 |
| ATOM | 1367 | CG2 | ILE | 167 | −18.413 | 10.964 | 19.459 | 1.00 | 0.56 | 1SG | 1368 |
| ATOM | 1368 | CG1 | ILE | 167 | −19.239 | 10.977 | 17.016 | 1.00 | 0.56 | 1SG | 1369 |
| ATOM | 1369 | CD1 | ILE | 167 | −20.704 | 10.598 | 17.213 | 1.00 | 0.56 | 1SG | 1370 |
| ATOM | 1370 | C | ILE | 167 | −16.495 | 11.905 | 17.771 | 1.00 | 0.56 | 1SG | 1371 |
| ATOM | 1371 | O | ILE | 167 | −16.832 | 12.719 | 16.914 | 1.00 | 0.56 | 1SG | 1372 |
| ATOM | 1372 | N | ASN | 168 | −15.691 | 12.203 | 18.817 | 1.00 | 0.50 | 1SG | 1373 |
| ATOM | 1373 | CA | ASN | 168 | −15.376 | 13.486 | 19.395 | 1.00 | 0.50 | 1SG | 1374 |
| ATOM | 1374 | CB | ASN | 168 | −14.920 | 14.653 | 18.460 | 1.00 | 0.50 | 1SG | 1375 |
| ATOM | 1375 | CG | ASN | 168 | −15.978 | 15.419 | 17.657 | 1.00 | 0.50 | 1SG | 1376 |
| ATOM | 1376 | OD1 | ASN | 168 | −16.681 | 16.289 | 18.168 | 1.00 | 0.50 | 1SG | 1377 |
| ATOM | 1377 | ND2 | ASN | 168 | −16.012 | 15.177 | 16.319 | 1.00 | 0.50 | 1SG | 1378 |
| ATOM | 1378 | C | ASN | 168 | −16.522 | 13.836 | 20.281 | 1.00 | 0.50 | 1SG | 1379 |
| ATOM | 1379 | O | ASN | 168 | −17.190 | 14.861 | 20.177 | 1.00 | 0.50 | 1SG | 1380 |
| ATOM | 1380 | N | LEU | 169 | −16.771 | 12.892 | 21.209 | 1.00 | 0.33 | 1SG | 1381 |
| ATOM | 1381 | CA | LEU | 169 | −17.881 | 12.949 | 22.107 | 1.00 | 0.33 | 1SG | 1382 |
| ATOM | 1382 | CB | LEU | 169 | −17.955 | 11.714 | 23.024 | 1.00 | 0.33 | 1SG | 1383 |
| ATOM | 1383 | CG | LEU | 169 | −19.137 | 11.762 | 24.007 | 1.00 | 0.33 | 1SG | 1384 |
| ATOM | 1384 | CD2 | LEU | 169 | −19.032 | 10.649 | 25.057 | 1.00 | 0.33 | 1SG | 1385 |
| ATOM | 1385 | CD1 | LEU | 169 | −20.483 | 11.756 | 23.256 | 1.00 | 0.33 | 1SG | 1386 |
| ATOM | 1386 | C | LEU | 169 | −17.863 | 14.153 | 23.005 | 1.00 | 0.33 | 1SG | 1387 |
| ATOM | 1387 | O | LEU | 169 | −18.822 | 14.923 | 23.015 | 1.00 | 0.33 | 1SG | 1388 |
| ATOM | 1388 | N | CYS | 170 | −16.765 | 14.367 | 23.758 | 1.00 | 0.21 | 1SG | 1389 |
| ATOM | 1389 | CA | CYS | 170 | −16.730 | 15.373 | 24.791 | 1.00 | 0.21 | 1SG | 1390 |
| ATOM | 1390 | CB | CYS | 170 | −15.503 | 15.247 | 25.705 | 1.00 | 0.21 | 1SG | 1391 |
| ATOM | 1391 | SG | CYS | 170 | −15.487 | 16.525 | 27.001 | 1.00 | 0.21 | 1SG | 1392 |
| ATOM | 1392 | C | CYS | 170 | −16.751 | 16.777 | 24.277 | 1.00 | 0.21 | 1SG | 1393 |
| ATOM | 1393 | O | CYS | 170 | −17.415 | 17.626 | 24.866 | 1.00 | 0.21 | 1SG | 1394 |
| ATOM | 1394 | N | GLY | 171 | −16.070 | 17.077 | 23.157 | 1.00 | 0.21 | 1SG | 1395 |
| ATOM | 1395 | CA | GLY | 171 | −15.972 | 18.464 | 22.791 | 1.00 | 0.21 | 1SG | 1396 |
| ATOM | 1396 | C | GLY | 171 | −14.548 | 18.875 | 22.990 | 1.00 | 0.21 | 1SG | 1397 |
| ATOM | 1397 | O | GLY | 171 | −14.226 | 20.047 | 23.173 | 1.00 | 0.21 | 1SG | 1398 |
| ATOM | 1398 | N | MET | 172 | −13.665 | 17.866 | 22.973 | 1.00 | 0.25 | 1SG | 1399 |
| ATOM | 1399 | CA | MET | 172 | −12.243 | 17.989 | 23.069 | 1.00 | 0.25 | 1SG | 1400 |
| ATOM | 1400 | CB | MET | 172 | −11.546 | 16.624 | 23.133 | 1.00 | 0.25 | 1SG | 1401 |
| ATOM | 1401 | CG | MET | 172 | −12.022 | 15.782 | 24.315 | 1.00 | 0.25 | 1SG | 1402 |
| ATOM | 1402 | SD | MET | 172 | −11.782 | 16.559 | 25.937 | 1.00 | 0.25 | 1SG | 1403 |
| ATOM | 1403 | CE | MET | 172 | −12.581 | 15.206 | 26.846 | 1.00 | 0.25 | 1SG | 1404 |
| ATOM | 1404 | C | MET | 172 | −11.800 | 18.646 | 21.805 | 1.00 | 0.25 | 1SG | 1405 |
| ATOM | 1405 | O | MET | 172 | −10.679 | 19.138 | 21.688 | 1.00 | 0.25 | 1SG | 1406 |
| ATOM | 1406 | N | ASP | 173 | −12.720 | 18.681 | 20.830 | 1.00 | 0.52 | 1SG | 1407 |
| ATOM | 1407 | CA | ASP | 173 | −12.456 | 19.003 | 19.464 | 1.00 | 0.52 | 1SG | 1408 |
| ATOM | 1408 | CB | ASP | 173 | −13.757 | 19.137 | 18.650 | 1.00 | 0.52 | 1SG | 1409 |
| ATOM | 1409 | CG | ASP | 173 | −13.407 | 19.065 | 17.173 | 1.00 | 0.52 | 1SG | 1410 |
| ATOM | 1410 | OD1 | ASP | 173 | −12.210 | 18.829 | 16.860 | 1.00 | 0.52 | 1SG | 1411 |
| ATOM | 1411 | OD2 | ASP | 173 | −14.331 | 19.247 | 16.336 | 1.00 | 0.52 | 1SG | 1412 |
| ATOM | 1412 | C | ASP | 173 | −11.647 | 20.255 | 19.257 | 1.00 | 0.52 | 1SG | 1413 |
| ATOM | 1413 | O | ASP | 173 | −10.641 | 20.177 | 18.557 | 1.00 | 0.52 | 1SG | 1414 |
| ATOM | 1414 | N | PHE | 174 | −11.969 | 21.427 | 19.851 | 1.00 | 0.77 | 1SG | 1415 |
| ATOM | 1415 | CA | PHE | 174 | −11.223 | 22.533 | 19.294 | 1.00 | 0.77 | 1SG | 1416 |
| ATOM | 1416 | CB | PHE | 174 | −12.034 | 23.396 | 18.324 | 1.00 | 0.77 | 1SG | 1417 |
| ATOM | 1417 | CG | PHE | 174 | −12.023 | 22.879 | 16.950 | 1.00 | 0.77 | 1SG | 1418 |
| ATOM | 1418 | CD1 | PHE | 174 | −12.895 | 21.899 | 16.547 | 1.00 | 0.77 | 1SG | 1419 |
| ATOM | 1419 | CD2 | PHE | 174 | −11.129 | 23.431 | 16.067 | 1.00 | 0.77 | 1SG | 1420 |
| ATOM | 1420 | CE1 | PHE | 174 | −12.865 | 21.449 | 15.251 | 1.00 | 0.77 | 1SG | 1421 |
| ATOM | 1421 | CE2 | PHE | 174 | −11.096 | 22.985 | 14.776 | 1.00 | 0.77 | 1SG | 1422 |
| ATOM | 1422 | CZ | PHE | 174 | −11.964 | 21.997 | 14.373 | 1.00 | 0.77 | 1SG | 1423 |
| ATOM | 1423 | C | PHE | 174 | −10.794 | 23.631 | 20.197 | 1.00 | 0.77 | 1SG | 1424 |
| ATOM | 1424 | O | PHE | 174 | −11.524 | 24.151 | 21.040 | 1.00 | 0.77 | 1SG | 1425 |
| ATOM | 1425 | N | PRO | 175 | −9.556 | 23.966 | 19.934 | 1.00 | 0.60 | 1SG | 1426 |
| ATOM | 1426 | CA | PRO | 175 | −9.055 | 25.262 | 20.295 | 1.00 | 0.60 | 1SG | 1427 |
| ATOM | 1427 | CD | PRO | 175 | −8.532 | 22.942 | 20.079 | 1.00 | 0.60 | 1SG | 1428 |
| ATOM | 1428 | CB | PRO | 175 | −7.555 | 25.106 | 20.527 | 1.00 | 0.60 | 1SG | 1429 |
| ATOM | 1429 | CG | PRO | 175 | −7.383 | 23.614 | 20.846 | 1.00 | 0.60 | 1SG | 1430 |
| ATOM | 1430 | C | PRO | 175 | −9.342 | 26.020 | 19.023 | 1.00 | 0.60 | 1SG | 1431 |
| ATOM | 1431 | O | PRO | 175 | −9.458 | 25.380 | 17.981 | 1.00 | 0.60 | 1SG | 1432 |
| ATOM | 1432 | N | ILE | 176 | −9.558 | 27.341 | 19.076 | 1.00 | 0.69 | 1SG | 1433 |
| ATOM | 1433 | CA | ILE | 176 | −9.821 | 28.157 | 17.920 | 1.00 | 0.69 | 1SG | 1434 |
| ATOM | 1434 | CB | ILE | 176 | −10.918 | 29.126 | 18.189 | 1.00 | 0.69 | 1SG | 1435 |
| ATOM | 1435 | CG2 | ILE | 176 | −12.190 | 28.298 | 18.444 | 1.00 | 0.69 | 1SG | 1436 |
| ATOM | 1436 | CG1 | ILE | 176 | −10.522 | 30.058 | 19.344 | 1.00 | 0.69 | 1SG | 1437 |
| ATOM | 1437 | CD1 | ILE | 176 | −11.471 | 31.234 | 19.530 | 1.00 | 0.69 | 1SG | 1438 |
| ATOM | 1438 | C | ILE | 176 | −8.639 | 28.889 | 17.351 | 1.00 | 0.69 | 1SG | 1439 |
| ATOM | 1439 | O | ILE | 176 | −8.772 | 29.517 | 16.304 | 1.00 | 0.69 | 1SG | 1440 |
| ATOM | 1440 | N | LYS | 177 | −7.490 | 28.932 | 18.048 | 1.00 | 0.91 | 1SG | 1441 |
| ATOM | 1441 | CA | LYS | 177 | −6.436 | 29.829 | 17.646 | 1.00 | 0.91 | 1SG | 1442 |
| ATOM | 1442 | CB | LYS | 177 | −5.786 | 30.542 | 18.844 | 1.00 | 0.91 | 1SG | 1443 |
| ATOM | 1443 | CG | LYS | 177 | −5.190 | 29.590 | 19.884 | 1.00 | 0.91 | 1SG | 1444 |
| ATOM | 1444 | CD | LYS | 177 | −6.219 | 28.676 | 20.554 | 1.00 | 0.91 | 1SG | 1445 |
| ATOM | 1445 | CE | LYS | 177 | −7.203 | 29.419 | 21.460 | 1.00 | 0.91 | 1SG | 1446 |
| ATOM | 1446 | NZ | LYS | 177 | −8.237 | 28.484 | 21.956 | 1.00 | 0.91 | 1SG | 1447 |
| ATOM | 1447 | C | LYS | 177 | −5.347 | 29.148 | 16.885 | 1.00 | 0.91 | 1SG | 1448 |
| ATOM | 1448 | O | LYS | 177 | −5.575 | 28.440 | 15.905 | 1.00 | 0.91 | 1SG | 1449 |
| ATOM | 1449 | N | THR | 178 | −4.096 | 29.470 | 17.264 | 1.00 | 0.60 | 1SG | 1450 |
| ATOM | 1450 | CA | THR | 178 | −2.965 | 28.861 | 16.638 | 1.00 | 0.60 | 1SG | 1451 |
| ATOM | 1451 | CB | THR | 178 | −1.893 | 29.839 | 16.259 | 1.00 | 0.60 | 1SG | 1452 |
| ATOM | 1452 | OG1 | THR | 178 | −1.383 | 30.481 | 17.418 | 1.00 | 0.60 | 1SG | 1453 |
| ATOM | 1453 | CG2 | THR | 178 | −2.494 | 30.878 | 15.297 | 1.00 | 0.60 | 1SG | 1454 |
| ATOM | 1454 | C | THR | 178 | −2.378 | 27.915 | 17.630 | 1.00 | 0.60 | 1SG | 1455 |
| ATOM | 1455 | O | THR | 178 | −2.869 | 27.814 | 16.754 | 1.00 | 0.60 | 1SG | 1456 |
| ATOM | 1456 | N | ASN | 179 | −1.311 | 27.201 | 17.206 | 1.00 | 0.42 | 1SG | 1457 |
| ATOM | 1457 | CA | ASN | 179 | −0.577 | 26.274 | 18.019 | 1.00 | 0.42 | 1SG | 1458 |
| ATOM | 1458 | CB | ASN | 179 | 0.415 | 26.977 | 18.965 | 1.00 | 0.42 | 1SG | 1459 |
| ATOM | 1459 | CG | ASN | 179 | 1.432 | 25.957 | 19.450 | 1.00 | 0.42 | 1SG | 1460 |
| ATOM | 1460 | OD1 | ASN | 179 | 2.628 | 26.097 | 19.196 | 1.00 | 0.42 | 1SG | 1461 |
| ATOM | 1461 | ND2 | ASN | 179 | 0.952 | 24.906 | 20.166 | 1.00 | 0.42 | 1SG | 1462 |
| ATOM | 1462 | C | ASN | 179 | −1.527 | 25.443 | 18.821 | 1.00 | 0.42 | 1SG | 1463 |
| ATOM | 1463 | O | ASN | 179 | −1.738 | 25.690 | 20.006 | 1.00 | 0.42 | 1SG | 1464 |
| ATOM | 1464 | N | LEU | 180 | −2.164 | 24.457 | 18.166 | 1.00 | 0.47 | 1SG | 1465 |
| ATOM | 1465 | CA | LEU | 180 | −3.137 | 23.617 | 18.801 | 1.00 | 0.47 | 1SG | 1466 |
| ATOM | 1466 | CB | LEU | 180 | −3.911 | 22.730 | 17.801 | 1.00 | 0.47 | 1SG | 1467 |
| ATOM | 1467 | CG | LEU | 180 | −3.047 | 21.892 | 16.836 | 1.00 | 0.47 | 1SG | 1468 |
| ATOM | 1468 | CD2 | LEU | 180 | −3.885 | 21.381 | 15.653 | 1.00 | 0.47 | 1SG | 1469 |
| ATOM | 1469 | CD1 | LEU | 180 | −2.298 | 20.763 | 17.558 | 1.00 | 0.47 | 1SG | 1470 |
| ATOM | 1470 | C | LEU | 180 | −2.495 | 22.765 | 19.854 | 1.00 | 0.47 | 1SG | 1471 |
| ATOM | 1471 | O | LEU | 180 | −3.099 | 22.503 | 20.891 | 1.00 | 0.47 | 1SG | 1472 |
| ATOM | 1472 | N | GLU | 181 | −1.236 | 22.345 | 19.638 | 1.00 | 0.40 | 1SG | 1473 |
| ATOM | 1473 | CA | GLU | 181 | −0.562 | 21.429 | 20.515 | 1.00 | 0.40 | 1SG | 1474 |
| ATOM | 1474 | CB | GLU | 181 | 0.836 | 21.038 | 20.011 | 1.00 | 0.40 | 1SG | 1475 |
| ATOM | 1475 | CG | GLU | 181 | 1.484 | 19.925 | 20.835 | 1.00 | 0.40 | 1SG | 1476 |
| ATOM | 1476 | CD | GLU | 181 | 2.611 | 19.332 | 20.003 | 1.00 | 0.40 | 1SG | 1477 |
| ATOM | 1477 | OE1 | GLU | 181 | 2.600 | 19.552 | 18.763 | 1.00 | 0.40 | 1SG | 1478 |
| ATOM | 1478 | OE2 | GLU | 181 | 3.491 | 18.649 | 20.591 | 1.00 | 0.40 | 1SG | 1479 |
| ATOM | 1479 | C | GLU | 181 | −0.436 | 22.026 | 21.882 | 1.00 | 0.40 | 1SG | 1480 |
| ATOM | 1480 | O | GLU | 181 | −0.338 | 21.316 | 22.880 | 1.00 | 0.40 | 1SG | 1481 |
| ATOM | 1481 | N | ILE | 182 | −0.420 | 23.358 | 21.967 | 1.00 | 0.45 | 1SG | 1482 |
| ATOM | 1482 | CA | ILE | 182 | −0.271 | 24.035 | 23.218 | 1.00 | 0.45 | 1SG | 1483 |
| ATOM | 1483 | CB | ILE | 182 | −0.215 | 25.523 | 23.030 | 1.00 | 0.45 | 1SG | 1484 |
| ATOM | 1484 | CG2 | ILE | 182 | −1.632 | 26.035 | 22.727 | 1.00 | 0.45 | 1SG | 1485 |
| ATOM | 1485 | CG1 | ILE | 182 | 0.434 | 26.194 | 24.242 | 1.00 | 0.45 | 1SG | 1486 |
| ATOM | 1486 | CD1 | ILE | 182 | 0.846 | 27.638 | 23.968 | 1.00 | 0.45 | 1SG | 1487 |
| ATOM | 1487 | C | ILE | 182 | −1.442 | 23.681 | 24.095 | 1.00 | 0.45 | 1SG | 1488 |
| ATOM | 1488 | O | ILE | 182 | −1.333 | 23.685 | 25.320 | 1.00 | 0.45 | 1SG | 1489 |
| ATOM | 1489 | N | VAL | 183 | −2.588 | 23.334 | 23.476 | 1.00 | 0.70 | 1SG | 1490 |
| ATOM | 1490 | CA | VAL | 183 | −3.836 | 23.042 | 24.139 | 1.00 | 0.70 | 1SG | 1491 |
| ATOM | 1491 | CB | VAL | 183 | −4.969 | 22.709 | 23.210 | 1.00 | 0.70 | 1SG | 1492 |
| ATOM | 1492 | CG1 | VAL | 183 | −4.777 | 21.280 | 22.675 | 1.00 | 0.70 | 1SG | 1493 |
| ATOM | 1493 | CG2 | VAL | 183 | −6.288 | 22.911 | 23.975 | 1.00 | 0.70 | 1SG | 1494 |
| ATOM | 1494 | C | VAL | 183 | −3.660 | 21.867 | 25.044 | 1.00 | 0.70 | 1SG | 1495 |
| ATOM | 1495 | O | VAL | 183 | −4.483 | 21.630 | 25.924 | 1.00 | 0.70 | 1SG | 1496 |
| ATOM | 1496 | N | ARG | 184 | −2.559 | 21.125 | 24.841 | 1.00 | 0.94 | 1SG | 1497 |
| ATOM | 1497 | CA | ARG | 184 | −2.154 | 19.912 | 25.494 | 1.00 | 0.94 | 1SG | 1498 |
| ATOM | 1498 | CB | ARG | 184 | −0.905 | 19.248 | 24.885 | 1.00 | 0.94 | 1SG | 1499 |
| ATOM | 1499 | CG | ARG | 184 | −1.231 | 18.358 | 23.679 | 1.00 | 0.94 | 1SG | 1500 |
| ATOM | 1500 | CD | ARG | 184 | −1.896 | 19.098 | 22.515 | 1.00 | 0.94 | 1SG | 1501 |
| ATOM | 1501 | NE | ARG | 184 | −2.140 | 18.121 | 21.417 | 1.00 | 0.94 | 1SG | 1502 |
| ATOM | 1502 | CZ | ARG | 184 | −3.308 | 17.418 | 21.370 | 1.00 | 0.94 | 1SG | 1503 |
| ATOM | 1503 | NH1 | ARG | 184 | −4.260 | 17.604 | 22.330 | 1.00 | 0.94 | 1SG | 1504 |
| ATOM | 1504 | NH2 | ARG | 184 | −3.529 | 16.527 | 20.362 | 1.00 | 0.94 | 1SG | 1505 |
| ATOM | 1505 | C | ARG | 184 | −1.943 | 20.129 | 26.959 | 1.00 | 0.94 | 1SG | 1506 |
| ATOM | 1506 | O | ARG | 184 | −1.222 | 19.364 | 27.593 | 1.00 | 0.94 | 1SG | 1507 |
| ATOM | 1507 | N | LYS | 185 | −2.392 | 21.277 | 27.498 | 1.00 | 0.95 | 1SG | 1508 |
| ATOM | 1508 | CA | LYS | 185 | −2.288 | 21.497 | 28.911 | 1.00 | 0.95 | 1SG | 1509 |
| ATOM | 1509 | CB | LYS | 185 | −2.757 | 22.891 | 29.363 | 1.00 | 0.95 | 1SG | 1510 |
| ATOM | 1510 | CG | LYS | 185 | −4.251 | 23.152 | 29.178 | 1.00 | 0.95 | 1SG | 1511 |
| ATOM | 1511 | CD | LYS | 185 | −4.710 | 24.448 | 29.846 | 1.00 | 0.95 | 1SG | 1512 |
| ATOM | 1512 | CE | LYS | 185 | −4.072 | 25.696 | 29.232 | 1.00 | 0.95 | 1SG | 1513 |
| ATOM | 1513 | NZ | LYS | 185 | −4.569 | 26.913 | 29.911 | 1.00 | 0.95 | 1SG | 1514 |
| ATOM | 1514 | C | LYS | 185 | −3.080 | 20.452 | 29.651 | 1.00 | 0.95 | 1SG | 1515 |
| ATOM | 1515 | O | LYS | 185 | −3.585 | 19.491 | 29.071 | 1.00 | 0.95 | 1SG | 1516 |
| ATOM | 1516 | N | LEU | 186 | −3.166 | 20.617 | 30.989 | 1.00 | 0.79 | 1SG | 1517 |
| ATOM | 1517 | CA | LEU | 186 | −3.770 | 19.674 | 31.888 | 1.00 | 0.79 | 1SG | 1518 |
| ATOM | 1518 | CB | LEU | 186 | −3.641 | 20.085 | 33.361 | 1.00 | 0.79 | 1SG | 1519 |
| ATOM | 1515 | CG | LEU | 186 | −2.188 | 20.066 | 33.855 | 1.00 | 0.79 | 1SG | 1520 |
| ATOM | 1520 | CD2 | LEU | 186 | −2.110 | 20.258 | 35.376 | 1.00 | 0.79 | 1SG | 1521 |
| ATOM | 1521 | CD1 | LEU | 136 | −1.334 | 21.079 | 33.084 | 1.00 | 0.79 | 1SG | 1522 |
| ATOM | 1522 | C | LEU | 186 | −5.225 | 19.495 | 31.613 | 1.00 | 0.79 | 1SG | 1523 |
| ATOM | 1523 | O | LEU | 186 | −5.729 | 18.374 | 31.662 | 1.00 | 0.79 | 1SG | 1524 |
| ATOM | 1524 | N | LYS | 187 | −5.943 | 20.590 | 31.316 | 1.00 | 0.61 | 1SG | 1525 |
| ATOM | 1525 | CA | LYS | 187 | −7.363 | 20.493 | 31.145 | 1.00 | 0.61 | 1SG | 1526 |
| ATOM | 1526 | CB | LYS | 187 | −7.958 | 21.801 | 30.594 | 1.00 | 0.61 | 1SG | 1527 |
| ATOM | 1527 | CG | LYS | 187 | −7.638 | 23.081 | 31.370 | 1.00 | 0.61 | 1SG | 1528 |
| ATOM | 1528 | CD | LYS | 187 | −8.311 | 23.218 | 32.734 | 1.00 | 0.61 | 1SG | 1529 |
| ATOM | 1529 | CE | LYS | 187 | −8.053 | 24.585 | 33.376 | 1.00 | 0.61 | 1SG | 1530 |
| ATOM | 1530 | NZ | LYS | 187 | −9.019 | 24.829 | 34.469 | 1.00 | 0.61 | 1SG | 1531 |
| ATOM | 1531 | C | LYS | 187 | −7.655 | 19.488 | 30.069 | 1.00 | 0.61 | 1SG | 1532 |
| ATOM | 1532 | O | LYS | 187 | −8.365 | 18.509 | 30.296 | 1.00 | 0.61 | 1SG | 1533 |
| ATOM | 1533 | N | CYS | 188 | −7.094 | 19.702 | 28.862 | 1.00 | 0.42 | 1SG | 1534 |
| ATOM | 1534 | CA | CYS | 188 | −7.391 | 18.827 | 27.765 | 1.00 | 0.42 | 1SG | 1535 |
| ATOM | 1535 | CB | CYS | 188 | −6.912 | 19.319 | 26.380 | 1.00 | 0.42 | 1SG | 1536 |
| ATOM | 1536 | SG | CYS | 188 | −5.202 | 18.870 | 25.940 | 1.00 | 0.42 | 1SG | 1537 |
| ATOM | 1537 | C | CYS | 188 | −6.762 | 17.501 | 28.018 | 1.00 | 0.42 | 1SG | 1538 |
| ATOM | 1538 | O | CYS | 188 | −7.373 | 16.463 | 27.772 | 1.00 | 0.42 | 1SG | 1539 |
| ATOM | 1539 | N | SER | 189 | −5.511 | 17.508 | 28.519 | 1.00 | 0.41 | 1SG | 1540 |
| ATOM | 1540 | CA | SER | 189 | −4.823 | 16.280 | 28.777 | 1.00 | 0.41 | 1SG | 1541 |
| ATOM | 1541 | CB | SER | 189 | −3.344 | 16.304 | 28.357 | 1.00 | 0.41 | 1SG | 1542 |
| ATOM | 1542 | OG | SER | 189 | −3.237 | 16.471 | 26.952 | 1.00 | 0.41 | 1SG | 1543 |
| ATOM | 1543 | C | SER | 189 | −4.854 | 16.116 | 30.254 | 1.00 | 0.41 | 1SG | 1544 |
| ATOM | 1544 | O | SER | 189 | −3.982 | 16.606 | 30.966 | 1.00 | 0.41 | 1SG | 1545 |
| ATOM | 1545 | N | THR | 190 | −5.845 | 15.355 | 30.732 | 1.00 | 0.53 | 1SG | 1546 |
| ATOM | 1546 | CA | THR | 190 | −6.126 | 15.182 | 32.122 | 1.00 | 0.53 | 1SG | 1547 |
| ATOM | 1547 | CB | THR | 190 | −7.369 | 14.375 | 32.371 | 1.00 | 0.53 | 1SG | 1548 |
| ATOM | 1548 | OG1 | THR | 190 | −7.222 | 13.059 | 31.858 | 1.00 | 0.53 | 1SG | 1549 |
| ATOM | 1549 | CG2 | THR | 190 | −8.554 | 15.080 | 31.689 | 1.00 | 0.53 | 1SG | 1550 |
| ATOM | 1550 | C | THR | 190 | −5.002 | 14.499 | 32.826 | 1.00 | 0.53 | 1SG | 1551 |
| ATOM | 1551 | O | THR | 190 | −4.825 | 14.718 | 34.022 | 1.00 | 0.53 | 1SG | 1552 |
| ATOM | 1552 | N | GLY | 191 | −4.137 | 13.747 | 32.119 | 1.00 | 0.49 | 1SG | 1553 |
| ATOM | 1553 | CA | GLY | 191 | −3.242 | 12.881 | 32.832 | 1.00 | 0.49 | 1SG | 1554 |
| ATOM | 1554 | C | GLY | 191 | −3.144 | 11.630 | 32.035 | 1.00 | 0.49 | 1SG | 1555 |
| ATOM | 1555 | O | GLY | 191 | −2.973 | 10.515 | 32.520 | 1.00 | 0.49 | 1SG | 1556 |
| ATOM | 1556 | N | GLU | 192 | −3.307 | 11.857 | 30.736 | 1.00 | 0.43 | 1SG | 1557 |
| ATOM | 1557 | CA | GLU | 192 | −3.146 | 10.967 | 29.646 | 1.00 | 0.43 | 1SG | 1558 |
| ATOM | 1558 | CB | GLU | 192 | −2.775 | 11.853 | 28.475 | 1.00 | 0.43 | 1SG | 1559 |
| ATOM | 1559 | CG | GLU | 192 | −1.816 | 11.228 | 27.511 | 1.00 | 0.43 | 1SG | 1560 |
| ATOM | 1560 | CD | GLU | 192 | −1.456 | 12.369 | 26.602 | 1.00 | 0.43 | 1SG | 1561 |
| ATOM | 1561 | OE1 | GLU | 192 | −2.034 | 13.473 | 26.781 | 1.00 | 0.43 | 1SG | 1562 |
| ATOM | 1562 | OE2 | GLU | 192 | −0.579 | 12.159 | 25.736 | 1.00 | 0.43 | 1SG | 1563 |
| ATOM | 1563 | C | GLU | 192 | −2.044 | 9.982 | 29.880 | 1.00 | 0.43 | 1SG | 1564 |
| ATOM | 1564 | O | GLU | 192 | −1.027 | 10.293 | 30.492 | 1.00 | 0.43 | 1SG | 1565 |
| ATOM | 1565 | N | ASN | 193 | −2.261 | 8.735 | 29.401 | 1.00 | 0.38 | 1SG | 1566 |
| ATOM | 1566 | CA | ASN | 193 | −1.263 | 7.702 | 29.452 | 1.00 | 0.38 | 1SG | 1567 |
| ATOM | 1567 | CB | ASN | 193 | −1.826 | 6.289 | 29.700 | 1.00 | 0.38 | 1SG | 1568 |
| ATOM | 1568 | CG | ASN | 193 | −2.363 | 6.199 | 31.132 | 1.00 | 0.38 | 1SG | 1569 |
| ATOM | 1569 | OD1 | ASN | 193 | −2.849 | 5.149 | 31.552 | 1.00 | 0.38 | 1SG | 1570 |
| ATOM | 1570 | ND2 | ASN | 193 | −2.284 | 7.322 | 31.898 | 1.00 | 0.38 | 1SG | 1571 |
| ATOM | 1571 | C | ASN | 193 | −0.660 | 7.732 | 28.087 | 1.00 | 0.38 | 1SG | 1572 |
| ATOM | 1572 | O | ASN | 193 | −1.248 | 8.312 | 27.187 | 1.00 | 0.38 | 1SG | 1573 |
| ATOM | 1573 | N | ASN | 194 | 0.536 | 7.143 | 27.896 | 1.00 | 0.31 | 1SG | 1574 |
| ATOM | 1574 | CA | ASN | 194 | 1.196 | 7.235 | 26.625 | 1.00 | 0.31 | 1SG | 1575 |
| ATOM | 1575 | CB | ASN | 194 | 2.158 | 8.437 | 26.547 | 1.00 | 0.31 | 1SG | 1576 |
| ATOM | 1576 | CG | ASN | 194 | 3.279 | 8.329 | 27.583 | 1.00 | 0.31 | 1SG | 1577 |
| ATOM | 1577 | OD1 | ASN | 194 | 4.367 | 8.822 | 27.311 | 1.00 | 0.31 | 1SG | 1578 |
| ATOM | 1578 | ND2 | ASN | 194 | 3.047 | 7.715 | 28.774 | 1.00 | 0.31 | 1SG | 1579 |
| ATOM | 1579 | C | ASN | 194 | 1.952 | 5.970 | 26.353 | 1.00 | 0.31 | 1SG | 1580 |
| ATOM | 1580 | O | ASN | 194 | 2.213 | 5.187 | 27.259 | 1.00 | 0.31 | 1SG | 1581 |
| ATOM | 1581 | N | LEU | 195 | 2.333 | 5.753 | 25.073 | 1.00 | 0.36 | 1SG | 1582 |
| ATOM | 1582 | CA | LEU | 195 | 2.923 | 4.528 | 24.592 | 1.00 | 0.36 | 1SG | 1583 |
| ATOM | 1583 | CB | LEU | 195 | 2.445 | 4.261 | 23.151 | 1.00 | 0.36 | 1SG | 1584 |
| ATOM | 1584 | CG | LEU | 195 | 2.481 | 2.824 | 22.642 | 1.00 | 0.36 | 1SG | 1585 |
| ATOM | 1585 | CD2 | LEU | 195 | 3.842 | 2.220 | 22.894 | 1.00 | 0.36 | 1SG | 1586 |
| ATOM | 1586 | CD1 | LEU | 195 | 2.025 | 2.739 | 21.176 | 1.00 | 0.36 | 1SG | 1587 |
| ATOM | 1587 | C | LEU | 195 | 4.407 | 4.737 | 24.484 | 1.00 | 0.36 | 1SG | 1588 |
| ATOM | 1588 | O | LEU | 195 | 4.852 | 5.807 | 24.082 | 1.00 | 0.36 | 1SG | 1589 |
| ATOM | 1589 | N | GLU | 196 | 5.206 | 3.715 | 24.858 | 1.00 | 0.49 | 1SG | 1590 |
| ATOM | 1590 | CA | GLU | 196 | 6.647 | 3.737 | 24.805 | 1.00 | 0.49 | 1SG | 1591 |
| ATOM | 1591 | CB | GLU | 196 | 7.255 | 2.544 | 25.558 | 1.00 | 0.49 | 1SG | 1592 |
| ATOM | 1592 | CG | GLU | 196 | 6.976 | 2.605 | 27.060 | 1.00 | 0.49 | 1SG | 1593 |
| ATOM | 1593 | CD | GLU | 196 | 7.225 | 1.229 | 27.649 | 1.00 | 0.49 | 1SG | 1594 |
| ATOM | 1594 | OE1 | GLU | 196 | 6.490 | 0.289 | 27.247 | 1.00 | 0.49 | 1SG | 1595 |
| ATOM | 1595 | OE2 | GLU | 196 | 8.145 | 1.092 | 28.501 | 1.00 | 0.49 | 1SG | 1596 |
| ATOM | 1596 | C | GLU | 196 | 7.154 | 3.716 | 23.384 | 1.00 | 0.49 | 1SG | 1597 |
| ATOM | 1597 | O | GLU | 196 | 8.151 | 4.353 | 23.061 | 1.00 | 0.49 | 1SG | 1598 |
| ATOM | 1598 | N | THR | 197 | 6.495 | 2.973 | 22.481 | 1.00 | 0.53 | 1SG | 1599 |
| ATOM | 1599 | CA | THR | 197 | 6.961 | 2.849 | 21.127 | 1.00 | 0.53 | 1SG | 1600 |
| ATOM | 1600 | CB | THR | 197 | 6.639 | 1.496 | 20.537 | 1.00 | 0.53 | 1SG | 1601 |
| ATOM | 1601 | OG1 | THR | 197 | 7.221 | 1.359 | 19.255 | 1.00 | 0.53 | 1SG | 1602 |
| ATOM | 1602 | CG2 | THR | 197 | 5.121 | 1.322 | 20.410 | 1.00 | 0.53 | 1SG | 1603 |
| ATOM | 1603 | C | THR | 197 | 6.321 | 3.916 | 20.278 | 1.00 | 0.53 | 1SG | 1604 |
| ATOM | 1604 | O | THR | 197 | 5.098 | 4.056 | 20.269 | 1.00 | 0.53 | 1SG | 1605 |
| ATOM | 1605 | N | GLU | 198 | 7.132 | 4.711 | 19.530 | 1.00 | 0.60 | 1SG | 1606 |
| ATOM | 1606 | CA | GLU | 198 | 6.509 | 5.750 | 18.749 | 1.00 | 0.60 | 1SG | 1607 |
| ATOM | 1607 | CB | GLU | 198 | 7.175 | 7.140 | 18.659 | 1.00 | 0.60 | 1SG | 1608 |
| ATOM | 1608 | CG | GLU | 198 | 8.404 | 7.256 | 17.760 | 1.00 | 0.60 | 1SG | 1609 |
| ATOM | 1609 | CD | GLU | 198 | 8.635 | 8.745 | 17.524 | 1.00 | 0.60 | 1SG | 1610 |
| ATOM | 1610 | OE1 | GLU | 198 | 8.621 | 9.514 | 18.521 | 1.00 | 0.60 | 1SG | 1611 |
| ATOM | 1611 | OE2 | GLU | 198 | 8.815 | 9.133 | 16.338 | 1.00 | 0.60 | 1SG | 1612 |
| ATOM | 1612 | C | GLU | 198 | 6.375 | 5.319 | 17.334 | 1.00 | 0.60 | 1SG | 1613 |
| ATOM | 1613 | O | GLU | 198 | 7.078 | 4.431 | 16.853 | 1.00 | 0.60 | 1SG | 1614 |
| ATOM | 1614 | N | LYS | 199 | 5.389 | 5.931 | 16.652 | 1.00 | 0.87 | 1SG | 1615 |
| ATOM | 1615 | CA | LYS | 199 | 5.135 | 5.611 | 15.284 | 1.00 | 0.87 | 1SG | 1616 |
| ATOM | 1616 | CB | LYS | 199 | 3.726 | 5.970 | 14.829 | 1.00 | 0.87 | 1SG | 1617 |
| ATOM | 1617 | CG | LYS | 199 | 2.775 | 4.967 | 15.474 | 1.00 | 0.87 | 1SG | 1618 |
| ATOM | 1618 | CD | LYS | 199 | 3.159 | 3.529 | 15.108 | 1.00 | 0.87 | 1SG | 1619 |
| ATOM | 1619 | CE | LYS | 199 | 2.566 | 2.455 | 16.021 | 1.00 | 0.87 | 1SG | 1620 |
| ATOM | 1620 | NZ | LYS | 199 | 3.445 | 2.236 | 17.191 | 1.00 | 0.87 | 1SG | 1621 |
| ATOM | 1621 | C | LYS | 199 | 6.191 | 6.129 | 14.346 | 1.00 | 0.87 | 1SG | 1622 |
| ATOM | 1622 | O | LYS | 199 | 6.539 | 5.356 | 13.454 | 1.00 | 0.87 | 1SG | 1623 |
| ATOM | 1623 | N | MET | 200 | 6.651 | 7.422 | 14.465 | 1.00 | 0.98 | 1SG | 1624 |
| ATOM | 1624 | CA | MET | 200 | 7.862 | 7.930 | 13.820 | 1.00 | 0.98 | 1SG | 1625 |
| ATOM | 1625 | CB | MET | 200 | 8.613 | 6.979 | 12.873 | 1.00 | 0.98 | 1SG | 1626 |
| ATOM | 1626 | CG | MET | 200 | 9.520 | 6.064 | 13.668 | 1.00 | 0.98 | 1SG | 1627 |
| ATOM | 1627 | SD | MET | 200 | 10.555 | 6.919 | 14.902 | 1.00 | 0.98 | 1SG | 1628 |
| ATOM | 1628 | CE | MET | 200 | 11.090 | 8.298 | 13.848 | 1.00 | 0.98 | 1SG | 1629 |
| ATOM | 1629 | C | MET | 200 | 8.031 | 9.439 | 13.498 | 1.00 | 0.98 | 1SG. | 1630 |
| ATOM | 1630 | O | MET | 200 | 7.334 | 10.292 | 14.044 | 1.00 | 0.98 | 1SG | 1631 |
| ATOM | 1631 | N | PRO | 201 | 8.968 | 9.735 | 12.576 | 1.00 | 0.72 | 1SG | 1632 |
| ATOM | 1632 | CA | PRO | 201 | 9.733 | 10.972 | 12.488 | 1.00 | 0.72 | 1SG | 1633 |
| ATOM | 1633 | CD | PRO | 201 | 8.689 | 9.385 | 11.183 | 1.00 | 0.72 | 1SG | 1634 |
| ATOM | 1634 | CB | PRO | 201 | 10.748 | 10.658 | 11.401 | 1.00 | 0.72 | 1SG | 1635 |
| ATOM | 1635 | CG | PRO | 201 | 9.934 | 9.829 | 10.392 | 1.00 | 0.72 | 1SG | 1636 |
| ATOM | 1636 | C | PRO | 201 | 9.331 | 12.412 | 12.295 | 1.00 | 0.72 | 1SG | 1637 |
| ATOM | 1637 | O | PRO | 201 | 9.882 | 13.162 | 13.089 | 1.00 | 0.72 | 1SG. | 1638 |
| ATOM | 1638 | N | PRO | 202 | 8.348 | 12.845 | 11.553 | 1.00 | 0.40 | 1SG | 1639 |
| ATOM | 1639 | CA | PRO | 202 | 8.384 | 14.188 | 10.939 | 1.00 | 0.40 | 1SG | 1640 |
| ATOM | 1640 | CD | PRO | 202 | 7.199 | 12.778 | 12.433 | 1.00 | 0.40 | 1SG | 1641 |
| ATOM | 1641 | CB | PRO | 202 | 7.134 | 14.927 | 11.379 | 1.00 | 0.40 | 1SG | 1642 |
| ATOM | 1642 | CG | PRO | 202 | 6.769 | 14.230 | 12.684 | 1.00 | 0.40 | 1SG | 1643 |
| ATOM | 1643 | C | PRO | 202 | 9.570 | 15.077 | 11.219 | 1.00 | 0.40 | 1SG | 1644 |
| ATOM | 1644 | O | PRO | 202 | 9.513 | 15.877 | 12.152 | 1.00 | 0.40 | 1SG | 1645 |
| ATOM | 1645 | N | ASN | 203 | 10.644 | 14.953 | 10.409 | 1.00 | 0.47 | 1SG | 1646 |
| ATOM | 1646 | CA | ASN | 203 | 11.844 | 15.721 | 10.593 | 1.00 | 0.47 | 1SG | 1647 |
| ATOM | 1647 | CB | ASN | 203 | 12.987 | 15.269 | 9.670 | 1.00 | 0.47 | 1SG | 1648 |
| ATOM | 1648 | CG | ASN | 203 | 13.416 | 13.871 | 10.092 | 1.00 | 0.47 | 1SG | 1649 |
| ATOM | 1649 | OD1 | ASN | 203 | 13.021 | 13.374 | 11.145 | 1.00 | 0.47 | 1SG | 1650 |
| ATOM | 1650 | ND2 | ASN | 203 | 14.257 | 13.218 | 9.246 | 1.00 | 0.47 | 1SG | 1651 |
| ATOM | 1651 | C | ASN | 203 | 11.589 | 17.166 | 10.296 | 1.00 | 0.47 | 1SG | 1652 |
| ATOM | 1652 | O | ASN | 203 | 11.910 | 18.038 | 11.102 | 1.00 | 0.47 | 1SG | 1653 |
| ATOM | 1653 | N | LYS | 204 | 10.981 | 17.458 | 9.129 | 1.00 | 0.74 | 1SG | 1654 |
| ATOM | 1654 | CA | LYS | 204 | 10.739 | 18.822 | 8.757 | 1.00 | 0.74 | 1SG | 1655 |
| ATOM | 1655 | CB | LYS | 204 | 11.082 | 19.157 | 7.297 | 1.00 | 0.74 | 1SG | 1656 |
| ATOM | 1656 | CG | LYS | 204 | 12.541 | 19.543 | 7.079 | 1.00 | 0.74 | 1SG | 1657 |
| ATOM | 1657 | CD | LYS | 204 | 12.923 | 20.838 | 7.796 | 1.00 | 0.74 | 1SG | 1658 |
| ATOM | 1658 | CE | LYS | 204 | 12.268 | 22.082 | 7.192 | 1.00 | 0.74 | 1SG | 1659 |
| ATOM | 1659 | NZ | LYS | 204 | 12.468 | 23.246 | 8.085 | 1.00 | 0.74 | 1SG | 1660 |
| ATOM | 1660 | C | LYS | 204 | 9.289 | 19.088 | 8.905 | 1.00 | 0.74 | 1SG | 1661 |
| ATOM | 1661 | O | LYS | 204 | 8.452 | 18.240 | 8.600 | 1.00 | 0.74 | 1SG | 1662 |
| ATOM | 1662 | N | GLU | 205 | 8.956 | 20.293 | 9.396 | 1.00 | 0.65 | 1SG | 1663 |
| ATOM | 1663 | CA | GLU | 205 | 7.577 | 20.605 | 9.569 | 1.00 | 0.65 | 1SG | 1664 |
| ATOM | 1664 | CB | GLU | 205 | 6.975 | 19.931 | 10.822 | 1.00 | 0.65 | 1SG | 1665 |
| ATOM | 1665 | CG | GLU | 205 | 5.443 | 19.910 | 10.892 | 1.00 | 0.65 | 1SG | 1666 |
| ATOM | 1666 | CD | GLU | 205 | 4.968 | 20.871 | 11.975 | 1.00 | 0.65 | 1SG | 1667 |
| ATOM | 1667 | OE1 | GLU | 205 | 5.840 | 21.460 | 12.669 | 1.00 | 0.65 | 1SG | 1668 |
| ATOM | 1668 | OE2 | GLU | 205 | 3.726 | 21.021 | 12.128 | 1.00 | 0.65 | 1SG | 1669 |
| ATOM | 1669 | C | GLU | 205 | 7.479 | 22.085 | 9.694 | 1.00 | 0.65 | 1SG | 1670 |
| ATOM | 1670 | O | GLU | 205 | 8.408 | 22.817 | 9.358 | 1.00 | 0.65 | 1SG | 1671 |
| ATOM | 1671 | N | GLU | 206 | 6.310 | 22.555 | 10.156 | 1.00 | 0.61 | 1SG | 1672 |
| ATOM | 1672 | CA | GLU | 206 | 6.061 | 23.950 | 10.336 | 1.00 | 0.61 | 1SG | 1673 |
| ATOM | 1673 | CB | GLU | 206 | 4.644 | 24.197 | 10.882 | 1.00 | 0.61 | 1SG | 1674 |
| ATOM | 1674 | CG | GLU | 206 | 4.263 | 25.669 | 11.025 | 1.00 | 0.61 | 1SG | 1675 |
| ATOM | 1675 | CD | GLU | 206 | 4.832 | 26.209 | 12.331 | 1.00 | 0.61 | 1SG | 1676 |
| ATOM | 1676 | OE1 | GLU | 206 | 5.241 | 25.385 | 13.191 | 1.00 | 0.61 | 1SG | 1677 |
| ATOM | 1677 | OE2 | GLU | 206 | 4.855 | 27.459 | 12.488 | 1.00 | 0.61 | 1SG | 1678 |
| ATOM | 1678 | C | GLU | 206 | 7.043 | 24.445 | 11.346 | 1.00 | 0.61 | 1SG | 1679 |
| ATOM | 1679 | O | GLU | 206 | 7.646 | 25.503 | 11.178 | 1.00 | 0.61 | 1SG. | 1680 |
| ATOM | 1680 | N | ARG | 207 | 7.243 | 23.660 | 12.421 | 1.00 | 0.63 | 1SG | 1681 |
| ATOM | 1681 | CA | ARG | 207 | 8.144 | 24.047 | 13.465 | 1.00 | 0.63 | 1SG | 1682 |
| ATOM | 1682 | CB | ARG | 207 | 8.114 | 23.143 | 14.705 | 1.00 | 0.63 | 1SG | 1683 |
| ATOM | 1683 | CG | ARG | 207 | 6.758 | 22.994 | 15.381 | 1.00 | 0.63 | 1SG | 1684 |
| ATOM | 1684 | CD | ARG | 207 | 6.875 | 22.270 | 16.722 | 1.00 | 0.63 | 1SG | 1685 |
| ATOM | 1685 | NE | ARG | 207 | 7.628 | 21.008 | 16.470 | 1.00 | 0.63 | 1SG | 1686 |
| ATOM | 1686 | CZ | ARG | 207 | 8.693 | 20.677 | 17.258 | 1.00 | 0.63 | 1SG | 1687 |
| ATOM | 1687 | NH1 | ARG | 207 | 9.058 | 21.487 | 18.294 | 1.00 | 0.63 | 1SG | 1688 |
| ATOM | 1688 | NH2 | ARG | 207 | 9.402 | 19.539 | 17.003 | 1.00 | 0.63 | 1SG | 1689 |
| ATOM | 1689 | C | ARG | 207 | 9.535 | 23.892 | 12.952 | 1.00 | 0.63 | 1SG | 1690 |
| ATOM | 1690 | O | ARG | 207 | 9.766 | 23.349 | 11.873 | 1.00 | 0.63 | 1SG | 1691 |
| ATOM | 1691 | N | TRP | 208 | 10.507 | 24.381 | 13.745 | 1.00 | 0.44 | 1SG | 1692 |
| ATOM | 1692 | CA | TRP | 208 | 11.884 | 24.287 | 13.370 | 1.00 | 0.44 | 1SG | 1693 |
| ATOM | 1693 | CB | TRP | 208 | 12.803 | 25.260 | 14.131 | 1.00 | 0.44 | 1SG | 1694 |
| ATOM | 1694 | CG | TRP | 208 | 12.598 | 26.712 | 13.772 | 1.00 | 0.44 | 1SG | 1695 |
| ATOM | 1695 | CD2 | TRP | 208 | 13.286 | 27.802 | 14.405 | 1.00 | 0.44 | 1SG | 1696 |
| ATOM | 1696 | CD1 | TRP | 208 | 11.775 | 27.267 | 12.835 | 1.00 | 0.44 | 1SG | 1697 |
| ATOM | 1697 | NE1 | TRP | 208 | 11.907 | 28.635 | 12.845 | 1.00 | 0.44 | 1SG | 1698 |
| ATOM | 1698 | CE2 | TRP | 208 | 12.834 | 28.978 | 13.807 | 1.00 | 0.44 | 1SG | 1699 |
| ATOM | 1699 | CE3 | TRP | 208 | 14.217 | 27.821 | 15.403 | 1.00 | 0.44 | 1SG | 1700 |
| ATOM | 1700 | CZ2 | TRP | 208 | 13.309 | 30.196 | 14.201 | 1.00 | 0.44 | 1SG | 1701 |
| ATOM | 1701 | CZ3 | TRP | 208 | 14.695 | 29.051 | 15.798 | 1.00 | 0.44 | 1SG | 1702 |
| ATOM | 1702 | CH2 | TRP | 208 | 14.249 | 30.215 | 15.208 | 1.00 | 0.44 | 1SG | 1703 |
| ATOM | 1703 | C | TRP | 208 | 12.364 | 22.899 | 13.651 | 1.00 | 0.44 | 1SG | 1704 |
| ATOM | 1704 | O | TRP | 208 | 11.807 | 22.179 | 14.480 | 1.00 | 0.44 | 1SG | 1705 |
| ATOM | 1705 | N | LYS | 209 | 13.417 | 22.499 | 12.913 | 1.00 | 0.42 | 1SG | 1706 |
| ATOM | 1706 | CA | LYS | 209 | 14.058 | 21.217 | 12.989 | 1.00 | 0.42 | 1SG | 1707 |
| ATOM | 1707 | CB | LYS | 209 | 15.077 | 20.984 | 11.863 | 1.00 | 0.42 | 1SG | 1708 |
| ATOM | 1708 | CG | LYS | 209 | 14.441 | 20.825 | 10.484 | 1.00 | 0.42 | 1SG | 1709 |
| ATOM | 1709 | CD | LYS | 209 | 15.440 | 20.941 | 9.330 | 1.00 | 0.42 | 1SG | 1710 |
| ATOM | 1710 | CE | LYS | 209 | 16.275 | 19.679 | 9.105 | 1.00 | 0.42 | 1SG | 1711 |
| ATOM | 1711 | NZ | LYS | 209 | 17.208 | 19.887 | 7.975 | 1.00 | 0.42 | 1SG | 1712 |
| ATOM | 1712 | C | LYS | 209 | 14.812 | 21.086 | 14.273 | 1.00 | 0.42 | 1SG | 1713 |
| ATOM | 1713 | O | LYS | 209 | 14.966 | 19.983 | 14.792 | 1.00 | 0.42 | 1SG | 1714 |
| ATOM | 1714 | N | LYS | 210 | 15.290 | 22.215 | 14.826 | 1.00 | 0.56 | 1SG | 1715 |
| ATOM | 1715 | CA | LYS | 210 | 16.187 | 22.189 | 15.947 | 1.00 | 0.56 | 1SG | 1716 |
| ATOM | 1716 | CB | LYS | 210 | 16.462 | 23.599 | 16.500 | 1.00 | 0.56 | 1SG | 1717 |
| ATOM | 1717 | CG | LYS | 210 | 17.416 | 23.626 | 17.696 | 1.00 | 0.56 | 1SG | 1718 |
| ATOM | 1718 | CD | LYS | 210 | 17.902 | 25.032 | 18.053 | 1.00 | 0.56 | 1SG | 1719 |
| ATOM | 1719 | CE | LYS | 210 | 16.810 | 25.915 | 18.660 | 1.00 | 0.56 | 1SG | 1720 |
| ATOM | 1720 | NZ | LYS | 210 | 17.356 | 27.253 | 18.976 | 1.00 | 0.56 | 1SG | 1721 |
| ATOM | 1721 | C | LYS | 210 | 15.623 | 21.372 | 17.068 | 1.00 | 0.56 | 1SG | 1722 |
| ATOM | 1722 | O | LYS | 210 | 16.328 | 20.531 | 17.626 | 1.00 | 0.56 | 1SG | 1723 |
| ATOM | 1723 | N | ARG | 211 | 14.342 | 21.570 | 17.423 | 1.00 | 0.69 | 1SG | 1724 |
| ATOM | 1724 | CA | ARG | 211 | 13.815 | 20.805 | 18.519 | 1.00 | 0.69 | 1SG | 1725 |
| ATOM | 1725 | CB | ARG | 211 | 12.324 | 21.057 | 18.792 | 1.00 | 0.69 | 1SG | 1726 |
| ATOM | 1726 | CG | ARG | 211 | 12.017 | 22.392 | 19.460 | 1.00 | 0.69 | 1SG | 1727 |
| ATOM | 1727 | CD | ARG | 211 | 12.184 | 22.374 | 20.980 | 1.00 | 0.69 | 1SG | 1728 |
| ATOM | 1728 | NE | ARG | 211 | 13.573 | 22.811 | 21.292 | 1.00 | 0.69 | 1SG | 1729 |
| ATOM | 1729 | CZ | ARG | 211 | 14.495 | 21.917 | 21.750 | 1.00 | 0.69 | 1SG | 1730 |
| ATOM | 1730 | NH1 | ARG | 211 | 14.166 | 20.597 | 21.860 | 1.00 | 0.69 | 1SG | 1731 |
| ATOM | 1731 | NH2 | ARG | 211 | 15.732 | 22.354 | 22.127 | 1.00 | 0.69 | 1SG | 1732 |
| ATOM | 1732 | C | ARG | 211 | 13.905 | 19.362 | 18.159 | 1.00 | 0.69 | 1SG | 1733 |
| ATOM | 1733 | O | ARG | 211 | 14.318 | 18.531 | 18.964 | 1.00 | 0.69 | 1SG | 1734 |
| ATOM | 1734 | N | TYR | 212 | 13.528 | 19.027 | 16.917 | 1.00 | 0.58 | 1SG | 1735 |
| ATOM | 1735 | CA | TYR | 212 | 13.511 | 17.655 | 16.509 | 1.00 | 0.58 | 1SG | 1736 |
| ATOM | 1736 | CB | TYR | 212 | 13.013 | 17.466 | 15.064 | 1.00 | 0.58 | 1SG | 1737 |
| ATOM | 1737 | CG | TYR | 212 | 13.052 | 16.004 | 14.771 | 1.00 | 0.58 | 1SG | 1738 |
| ATOM | 1738 | CD1 | TYR | 212 | 14.176 | 15.401 | 14.255 | 1.00 | 0.58 | 1SG | 1739 |
| ATOM | 1739 | CD2 | TYR | 212 | 11.949 | 15.227 | 15.027 | 1.00 | 0.58 | 1SG | 1740 |
| ATOM | 1740 | CE1 | TYR | 212 | 14.195 | 14.050 | 13.995 | 1.00 | 0.58 | 1SG | 1741 |
| ATOM | 1741 | CE2 | TYR | 212 | 11.962 | 13.878 | 14.768 | 1.00 | 0.58 | 1SG | 1742 |
| ATOM | 1742 | CZ | TYR | 212 | 13.086 | 13.284 | 14.251 | 1.00 | 0.58 | 1SG | 1743 |
| ATOM | 1743 | OH | TYR | 212 | 13.100 | 11.898 | 13.986 | 1.00 | 0.58 | 1SG | 1744 |
| ATOM | 1744 | C | TYR | 212 | 14.896 | 17.101 | 16.552 | 1.00 | 0.58 | 1SG | 1745 |
| ATOM | 1745 | O | TYR | 212 | 15.115 | 15.988 | 17.029 | 1.00 | 0.58 | 1SG | 1746 |
| ATOM | 1746 | N | ALA | 213 | 15.874 | 17.881 | 16.066 | 1.00 | 0.38 | 1SG | 1747 |
| ATOM | 1747 | CA | ALA | 213 | 17.215 | 17.394 | 15.957 | 1.00 | 0.38 | 1SG | 1748 |
| ATOM | 1748 | CB | ALA | 213 | 18.172 | 18.428 | 15.339 | 1.00 | 0.38 | 1SG | 1749 |
| ATOM | 1749 | C | ALA | 213 | 17.731 | 17.053 | 17.314 | 1.00 | 0.38 | 1SG | 1750 |
| ATOM | 1750 | O | ALA | 213 | 18.394 | 16.033 | 17.491 | 1.00 | 0.38 | 1SG | 1751 |
| ATOM | 1751 | N | VAL | 214 | 17.423 | 17.888 | 18.321 | 1.00 | 0.43 | 1SG | 1752 |
| ATOM | 1752 | CA | VAL | 214 | 17.956 | 17.640 | 19.626 | 1.00 | 0.43 | 1SG | 1753 |
| ATOM | 1753 | CB | VAL | 214 | 17.712 | 18.746 | 20.617 | 1.00 | 0.43 | 1SG | 1754 |
| ATOM | 1754 | CG1 | VAL | 214 | 18.334 | 20.044 | 20.076 | 1.00 | 0.43 | 1SG | 1755 |
| ATOM | 1755 | CG2 | VAL | 214 | 16.212 | 18.832 | 20.917 | 1.00 | 0.43 | 1SG | 1756 |
| ATOM | 1756 | C | VAL | 214 | 17.321 | 16.403 | 20.173 | 1.00 | 0.43 | 1SG | 1757 |
| ATOM | 1757 | O | VAL | 214 | 16.837 | 15.544 | 19.438 | 1.00 | 0.43 | 1SG | 1758 |
| ATOM | 1758 | N | VAL | 215 | 17.345 | 16.292 | 21.512 | 1.00 | 0.55 | 1SG | 1759 |
| ATOM | 1759 | CA | VAL | 215 | 16.841 | 15.174 | 22.253 | 1.00 | 0.55 | 1SG | 1760 |
| ATOM | 1760 | CB | VAL | 215 | 17.079 | 15.309 | 23.728 | 1.00 | 0.55 | 1SG | 1761 |
| ATOM | 1761 | CG1 | VAL | 215 | 16.305 | 16.536 | 24.240 | 1.00 | 0.55 | 1SG | 1762 |
| ATOM | 1762 | CG2 | VAL | 215 | 16.681 | 13.991 | 24.412 | 1.00 | 0.55 | 1SG | 1763 |
| ATOM | 1763 | C | VAL | 215 | 15.367 | 15.064 | 22.035 | 1.00 | 0.55 | 1SG | 1764 |
| ATOM | 1764 | O | VAL | 215 | 14.796 | 13.983 | 22.166 | 1.00 | 0.55 | 1SG | 1765 |
| ATOM | 1765 | N | ASP | 216 | 14.713 | 16.189 | 21.695 | 1.00 | 0.50 | 1SG | 1766 |
| ATOM | 1766 | CA | ASP | 216 | 13.285 | 16.216 | 21.559 | 1.00 | 0.50 | 1SG | 1767 |
| ATOM | 1767 | CB | ASP | 216 | 12.792 | 17.582 | 21.035 | 1.00 | 0.50 | 1SG | 1768 |
| ATOM | 1768 | CG | ASP | 216 | 11.275 | 17.673 | 21.136 | 1.00 | 0.50 | 1SG | 1769 |
| ATOM | 1769 | OD1 | ASP | 216 | 10.599 | 16.619 | 20.998 | 1.00 | 0.50 | 1SG | 1770 |
| ATOM | 1770 | OD2 | ASP | 216 | 10.773 | 18.808 | 21.355 | 1.00 | 0.50 | 1SG | 1771 |
| ATOM | 1771 | C | ASP | 216 | 12.881 | 15.152 | 20.590 | 1.00 | 0.50 | 1SG | 1772 |
| ATOM | 1772 | O | ASP | 216 | 11.938 | 14.404 | 20.844 | 1.00 | 0.50 | 1SG | 1773 |
| ATOM | 1773 | N | GLY | 217 | 13.598 | 15.029 | 19.460 | 1.00 | 0.56 | 1SG | 1774 |
| ATOM | 1774 | CA | GLY | 217 | 13.222 | 14.016 | 18.523 | 1.00 | 0.56 | 1SG | 1775 |
| ATOM | 1775 | C | GLY | 217 | 14.476 | 13.384 | 18.026 | 1.00 | 0.56 | 1SG | 1776 |
| ATOM | 1776 | O | GLY | 217 | 15.536 | 13.515 | 18.633 | 1.00 | 0.56 | 1SG | 1777 |
| ATOM | 1777 | N | LYS | 218 | 14.338 | 12.630 | 16.921 | 1.00 | 0.61 | 1SG | 1778 |
| ATOM | 1778 | CA | LYS | 218 | 15.368 | 11.927 | 16.211 | 1.00 | 0.61 | 1SG | 1779 |
| ATOM | 1779 | CB | LYS | 218 | 16.638 | 12.752 | 15.915 | 1.00 | 0.61 | 1SG | 1780 |
| ATOM | 1780 | CG | LYS | 218 | 17.659 | 12.810 | 17.054 | 1.00 | 0.61 | 1SG | 1781 |
| ATOM | 1781 | CD | LYS | 218 | 19.029 | 13.325 | 16.607 | 1.00 | 0.61 | 1SG | 1782 |
| ATOM | 1782 | CE | LYS | 218 | 20.115 | 13.220 | 17.681 | 1.00 | 0.61 | 1SG | 1783 |
| ATOM | 1783 | NZ | LYS | 218 | 19.821 | 14.143 | 18.799 | 1.00 | 0.61 | 1SG | 1784 |
| ATOM | 1784 | C | LYS | 218 | 15.752 | 10.738 | 17.031 | 1.00 | 0.61 | 1SG | 1785 |
| ATOM | 1785 | O | LYS | 218 | 15.934 | 9.640 | 16.508 | 1.00 | 0.61 | 1SG | 1786 |
| ATOM | 1786 | N | LEU | 219 | 15.816 | 10.925 | 18.362 | 1.00 | 0.51 | 1SG | 1787 |
| ATOM | 1787 | CA | LEU | 219 | 16.168 | 9.883 | 19.275 | 1.00 | 0.51 | 1SG | 1788 |
| ATOM | 1788 | CB | LEU | 219 | 16.306 | 10.374 | 20.725 | 1.00 | 0.51 | 1SG | 1789 |
| ATOM | 1789 | CG | LEU | 219 | 17.292 | 11.543 | 20.892 | 1.00 | 0.51 | 1SG | 1790 |
| ATOM | 1790 | CD2 | LEU | 219 | 18.524 | 11.385 | 19.992 | 1.00 | 0.51 | 1SG | 1791 |
| ATOM | 1791 | CD1 | LEU | 219 | 17.626 | 11.779 | 22.373 | 1.00 | 0.51 | 1SG | 1792 |
| ATOM | 1792 | C | LEU | 219 | 15.053 | 8.897 | 19.273 | 1.00 | 0.51 | 1SG | 1793 |
| ATOM | 1793 | O | LEU | 219 | 15.283 | 7.692 | 19.367 | 1.00 | 0.51 | 1SG | 1794 |
| ATOM | 1794 | N | THR | 220 | 13.805 | 9.408 | 19.171 | 1.00 | 0.58 | 1SG | 1795 |
| ATOM | 1795 | CA | THR | 220 | 12.651 | 8.556 | 19.206 | 1.00 | 0.58 | 1SG | 1796 |
| ATOM | 1796 | CB | THR | 220 | 11.352 | 9.247 | 18.887 | 1.00 | 0.58 | 1SG | 1797 |
| ATOM | 1797 | OG1 | THR | 220 | 11.317 | 9.678 | 17.535 | 1.00 | 0.58 | 1SG | 1798 |
| ATOM | 1798 | CG2 | THR | 220 | 11.215 | 10.470 | 19.794 | 1.00 | 0.58 | 1SG | 1799 |
| ATOM | 1799 | C | THR | 220 | 12.843 | 7.519 | 18.158 | 1.00 | 0.58 | 1SG | 1800 |
| ATOM | 1800 | O | THR | 220 | 12.995 | 7.817 | 16.974 | 1.00 | 0.58 | 1SG | 1801 |
| ATOM | 1801 | N | ASN | 221 | 12.846 | 6.252 | 18.589 | 1.00 | 0.55 | 1SG | 1802 |
| ATOM | 1802 | CA | ASN | 221 | 13.104 | 5.188 | 17.676 | 1.00 | 0.55 | 1SG | 1803 |
| ATOM | 1803 | CB | ASN | 221 | 13.642 | 3.935 | 18.391 | 1.00 | 0.55 | 1SG | 1804 |
| ATOM | 1804 | CG | ASN | 221 | 14.383 | 3.076 | 17.383 | 1.00 | 0.55 | 1SG | 1805 |
| ATOM | 1805 | OD1 | ASN | 221 | 15.034 | 3.600 | 16.480 | 1.00 | 0.55 | 1SG | 1806 |
| ATOM | 1806 | ND2 | ASN | 221 | 14.287 | 1.728 | 17.537 | 1.00 | 0.55 | 1SG | 1807 |
| ATOM | 1807 | C | ASN | 221 | 11.827 | 4.815 | 17.001 | 1.00 | 0.55 | 1SG | 1808 |
| ATOM | 1808 | O | ASN | 221 | 10.737 | 5.008 | 17.537 | 1.00 | 0.55 | 1SG | 1809 |
| ATOM | 1809 | N | THR | 222 | 11.956 | 4.277 | 15.775 | 1.00 | 0.31 | 1SG | 1810 |
| ATOM | 1810 | CA | THR | 222 | 10.843 | 3.810 | 15.008 | 1.00 | 0.31 | 1SG | 1811 |
| ATOM | 1811 | CB | THR | 222 | 11.213 | 3.520 | 13.580 | 1.00 | 0.31 | 1SG | 1812 |
| ATOM | 1812 | OG1 | THR | 222 | 11.798 | 4.656 | 12.961 | 1.00 | 0.31 | 1SG | 1813 |
| ATOM | 1813 | CG2 | THR | 222 | 9.948 | 3.086 | 12.823 | 1.00 | 0.31 | 1SG | 1814 |
| ATOM | 1814 | C | THR | 222 | 10.513 | 2.475 | 15.569 | 1.00 | 0.31 | 1SG | 1815 |
| ATOM | 1815 | O | THR | 222 | 11.386 | 1.811 | 16.126 | 1.00 | 0.31 | 1SG | 1816 |
| ATOM | 1816 | N | GLY | 223 | 9.241 | 2.048 | 15.465 | 1.00 | 0.40 | 1SG | 1817 |
| ATOM | 1817 | CA | GLY | 223 | 8.988 | 0.702 | 15.872 | 1.00 | 0.40 | 1SG | 1818 |
| ATOM | 1818 | C | GLY | 223 | 9.829 | −0.095 | 14.932 | 1.00 | 0.40 | 1SG | 1819 |
| ATOM | 1819 | O | GLY | 223 | 9.714 | 0.042 | 13.715 | 1.00 | 0.40 | 1SG | 1820 |
| ATOM | 1820 | N | ILE | 224 | 10.688 | −0.967 | 15.485 | 1.00 | 0.69 | 1SG | 1821 |
| ATOM | 1821 | CA | ILE | 224 | 11.634 | −1.698 | 14.697 | 1.00 | 0.69 | 1SG | 1822 |
| ATOM | 1822 | CB | ILE | 224 | 12.486 | −2.619 | 15.528 | 1.00 | 0.69 | 1SG | 1823 |
| ATOM | 1823 | CG2 | ILE | 224 | 11.590 | −3.778 | 15.993 | 1.00 | 0.69 | 1SG | 1824 |
| ATOM | 1824 | CG1 | ILE | 224 | 13.740 | −3.077 | 14.762 | 1.00 | 0.69 | 1SG | 1825 |
| ATOM | 1825 | CD1 | ILE | 224 | 14.729 | −1.949 | 14.476 | 1.00 | 0.69 | 1SG | 1826 |
| ATOM | 1826 | C | ILE | 224 | 10.896 | −2.519 | 13.693 | 1.00 | 0.69 | 1SG | 1827 |
| ATOM | 1827 | O | ILE | 224 | 9.669 | −2.496 | 13.617 | 1.00 | 0.69 | 1SG | 1828 |
| ATOM | 1828 | N | VAL | 225 | 11.655 | −3.268 | 12.876 | 1.00 | 0.69 | 1SG | 1829 |
| ATOM | 1829 | CA | VAL | 225 | 11.085 | −4.083 | 11.852 | 1.00 | 0.69 | 1SG | 1830 |
| ATOM | 1830 | CB | VAL | 225 | 12.110 | −4.858 | 11.078 | 1.00 | 0.69 | 1SG | 1831 |
| ATOM | 1831 | CG1 | VAL | 225 | 12.842 | −5.801 | 12.047 | 1.00 | 0.69 | 1SG | 1832 |
| ATOM | 1832 | CG2 | VAL | 225 | 11.401 | −5.583 | 9.921 | 1.00 | 0.69 | 1SG | 1833 |
| ATOM | 1833 | C | VAL | 225 | 10.191 | −5.064 | 12.521 | 1.00 | 0.69 | 1SG | 1834 |
| ATOM | 1834 | O | VAL | 225 | 9.142 | −5.417 | 11.985 | 1.00 | 0.69 | 1SG | 1835 |
| ATOM | 1835 | N | LYS | 226 | 10.576 | −5.538 | 13.716 | 1.00 | 0.47 | 1SG | 1836 |
| ATOM | 1836 | CA | LYS | 226 | 9.745 | −6.500 | 14.373 | 1.00 | 0.47 | 1SG | 1837 |
| ATOM | 1837 | CB | LYS | 226 | 10.342 | −7.033 | 15.685 | 1.00 | 0.47 | 1SG | 1838 |
| ATOM | 1838 | CG | LYS | 226 | 11.598 | −7.881 | 15.474 | 1.00 | 0.47 | 1SG | 1839 |
| ATOM | 1839 | CD | LYS | 226 | 11.357 | −9.129 | 14.623 | 1.00 | 0.47 | 1SG | 1840 |
| ATOM | 1840 | CE | LYS | 226 | 12.613 | −9.976 | 14.410 | 1.00 | 0.47 | 1SG | 1841 |
| ATOM | 1841 | NZ | LYS | 226 | 12.292 | −11.160 | 13.579 | 1.00 | 0.47 | 1SG | 1842 |
| ATOM | 1842 | C | LYS | 226 | 8.439 | −5.855 | 14.687 | 1.00 | 0.47 | 1SG | 1843 |
| ATOM | 1843 | O | LYS | 226 | 8.384 | −4.798 | 15.315 | 1.00 | 0.47 | 1SG | 1844 |
| ATOM | 1844 | N | ALA | 227 | 7.339 | −6.474 | 14.220 | 1.00 | 0.41 | 1SG | 1845 |
| ATOM | 1845 | CA | ALA | 227 | 6.052 | −5.926 | 14.522 | 1.00 | 0.41 | 1SG | 1846 |
| ATOM | 1846 | CB | ALA | 227 | 4.940 | −6.333 | 13.540 | 1.00 | 0.41 | 1SG | 1847 |
| ATOM | 1847 | C | ALA | 227 | 5.654 | −6.458 | 15.852 | 1.00 | 0.41 | 1SG | 1848 |
| ATOM | 1848 | O | ALA | 227 | 5.776 | −7.657 | 16.097 | 1.00 | 0.41 | 1SG | 1849 |
| ATOM | 1849 | N | PRO | 228 | 5.276 | −5.592 | 16.752 | 1.00 | 0.60 | 1SG | 1850 |
| ATOM | 1850 | CA | PRO | 228 | 4.769 | −6.131 | 17.982 | 1.00 | 0.60 | 1SG | 1851 |
| ATOM | 1851 | CD | PRO | 228 | 6.136 | −4.443 | 16.994 | 1.00 | 0.60 | 1SG | 1852 |
| ATOM | 1852 | CB | PRO | 228 | 5.171 | −5.162 | 19.091 | 1.00 | 0.60 | 1SG | 1853 |
| ATOM | 1853 | CG | PRO | 228 | 6.388 | −4.425 | 18.510 | 1.00 | 0.60 | 1SG | 1854 |
| ATOM | 1854 | C | PRO | 228 | 3.288 | −6.331 | 17.899 | 1.00 | 0.60 | 1SG | 1855 |
| ATOM | 1855 | O | PRO | 228 | 2.588 | −5.321 | 17.831 | 1.00 | 0.60 | 1SG | 1856 |
| ATOM | 1856 | N | PRO | 229 | 2.763 | −7.521 | 17.943 | 1.00 | 0.71 | 1SG | 1857 |
| ATOM | 1857 | CA | PRO | 229 | 1.332 | −7.594 | 17.975 | 1.00 | 0.71 | 1SG | 1858 |
| ATOM | 1858 | CD | PRO | 229 | 3.332 | −8.598 | 17.148 | 1.00 | 0.71 | 1SG | 1859 |
| ATOM | 1859 | CB | PRO | 229 | 0.954 | −8.973 | 17.441 | 1.00 | 0.71 | 1SG | 1860 |
| ATOM | 1860 | CG | PRO | 229 | 2.136 | −9.341 | 16.529 | 1.00 | 0.71 | 1SG | 1861 |
| ATOM | 1861 | C | PRO | 229 | 0.918 | −7.365 | 19.386 | 1.00 | 0.71 | 1SG | 1862 |
| ATOM | 1862 | O | PRO | 229 | 1.610 | −7.864 | 20.272 | 1.00 | 0.71 | 1SG | 1863 |
| ATOM | 1863 | N | PRO | 230 | −0.127 | −6.634 | 19.642 | 1.00 | 0.69 | 1SG | 1864 |
| ATOM | 1864 | CA | PRO | 230 | −0.499 | −6.519 | 21.020 | 1.00 | 0.69 | 1SG | 1865 |
| ATOM | 1865 | CD | PRO | 230 | −0.304 | −5.374 | 18.936 | 1.00 | 0.69 | 1SG | 1866 |
| ATOM | 1866 | CB | PRO | 230 | −1.248 | −5.199 | 21.162 | 1.00 | 0.69 | 1SG | 1867 |
| ATOM | 1867 | CG | PRO | 230 | −0.688 | −4.345 | 20.012 | 1.00 | 0.69 | 1SG | 1868 |
| ATOM | 1868 | C | PRO | 230 | −1.283 | −7.717 | 21.430 | 1.00 | 0.69 | 1SG | 1869 |
| ATOM | 1869 | O | PRO | 230 | −2.272 | −8.032 | 20.769 | 1.00 | 0.69 | 1SG | 1870 |
| ATOM | 1870 | N | LEU | 231 | −0.888 | −8.358 | 22.545 | 1.00 | 0.82 | 1SG | 1871 |
| ATOM | 1871 | CA | LEU | 231 | −1.555 | −9.538 | 23.003 | 1.00 | 0.82 | 1SG | 1872 |
| ATOM | 1872 | CB | LEU | 231 | −0.584 | −10.621 | 23.507 | 1.00 | 0.82 | 1SG | 1873 |
| ATOM | 1873 | CG | LEU | 231 | 0.434 | −11.043 | 22.423 | 1.00 | 0.82 | 1SG | 1874 |
| ATOM | 1874 | CD2 | LEU | 231 | 1.430 | −9.916 | 22.104 | 1.00 | 0.82 | 1SG | 1875 |
| ATOM | 1875 | CD1 | LEU | 231 | −0.276 | −11.578 | 21.167 | 1.00 | 0.82 | 1SG | 1876 |
| ATOM | 1876 | C | LEU | 231 | −2.426 | −9.106 | 24.128 | 1.00 | 0.82 | 1SG | 1877 |
| ATOM | 1877 | O | LEU | 231 | −2.798 | −7.936 | 24.206 | 1.00 | 0.82 | 1SG | 1878 |
| ATOM | 1878 | N | LYS | 232 | −2.800 | −10.045 | 25.018 | 1.00 | 0.92 | 1SG | 1879 |
| ATOM | 1879 | CA | LYS | 232 | −3.628 | −9.652 | 26.116 | 1.00 | 0.92 | 1SG | 1880 |
| ATOM | 1880 | CB | LYS | 232 | −4.042 | −10.820 | 27.031 | 1.00 | 0.92 | 1SG | 1881 |
| ATOM | 1881 | CG | LYS | 232 | −2.891 | −11.666 | 27.577 | 1.00 | 0.92 | 1SG | 1882 |
| ATOM | 1882 | CD | LYS | 232 | −3.366 | −12.743 | 28.555 | 1.00 | 0.92 | 1SG | 1883 |
| ATOM | 1883 | CE | LYS | 232 | −4.558 | −13.545 | 28.024 | 1.00 | 0.92 | 1SG | 1884 |
| ATOM | 1884 | NZ | LYS | 232 | −4.235 | −14.122 | 26.698 | 1.00 | 0.92 | 1SG | 1885 |
| ATOM | 1885 | C | LYS | 232 | −2.868 | −8.608 | 26.860 | 1.00 | 0.92 | 1SG | 1886 |
| ATOM | 1886 | O | LYS | 232 | −1.638 | −8.609 | 26.866 | 1.00 | 0.92 | 1SG | 1887 |
| ATOM | 1887 | N | THR | 233 | −3.576 | −7.639 | 27.468 | 1.00 | 0.79 | 1SG | 1888 |
| ATOM | 1888 | CA | THR | 233 | −2.790 | −6.599 | 28.047 | 1.00 | 0.79 | 1SG | 1889 |
| ATOM | 1889 | CB | THR | 233 | −2.806 | −5.346 | 27.227 | 1.00 | 0.79 | 1SG | 1890 |
| ATOM | 1890 | OG1 | THR | 233 | −1.629 | −4.597 | 27.468 | 1.00 | 0.79 | 1SG | 1891 |
| ATOM | 1891 | CG2 | THR | 233 | −4.033 | −4.511 | 27.621 | 1.00 | 0.79 | 1SG | 1892 |
| ATOM | 1892 | C | THR | 233 | −3.298 | −6.284 | 29.416 | 1.00 | 0.79 | 1SG | 1893 |
| ATOM | 1893 | O | THR | 233 | −4.498 | −6.267 | 29.686 | 1.00 | 0.79 | 1SG | 1894 |
| ATOM | 1894 | N | PRO | 234 | −2.360 | −6.085 | 30.297 | 1.00 | 0.58 | 1SG | 1895 |
| ATOM | 1895 | CA | PRO | 234 | −2.661 | −5.675 | 31.639 | 1.00 | 0.58 | 1SG | 1896 |
| ATOM | 1896 | CD | PRO | 234 | −1.082 | −6.769 | 30.209 | 1.00 | 0.58 | 1SG | 1897 |
| ATOM | 1897 | CB | PRO | 234 | −1.411 | −5.975 | 32.469 | 1.00 | 0.58 | 1SG | 1898 |
| ATOM | 1898 | CG | PRO | 234 | −0.308 | −6.264 | 31.434 | 1.00 | 0.58 | 1SG | 1899 |
| ATOM | 1899 | C | PRO | 234 | −3.049 | −4.234 | 31.615 | 1.00 | 0.58 | 1SG | 1900 |
| ATOM | 1900 | O | PRO | 234 | −3.593 | −3.744 | 32.602 | 1.00 | 0.58 | 1SG | 1901 |
| ATOM | 1901 | N | LEU | 235 | −2.754 | −3.547 | 30.498 | 1.00 | 0.33 | 1SG | 1902 |
| ATOM | 1902 | CA | LEU | 235 | −3.054 | −2.163 | 30.303 | 1.00 | 0.33 | 1SG | 1903 |
| ATOM | 1903 | CB | LEU | 235 | −2.532 | −1.602 | 28.971 | 1.00 | 0.33 | 1SG | 1904 |
| ATOM | 1904 | CG | LEU | 235 | −1.005 | −1.686 | 28.791 | 1.00 | 0.33 | 1SG | 1905 |
| ATOM | 1905 | CD2 | LEU | 235 | −0.250 | −1.173 | 30.026 | 1.00 | 0.33 | 1SG | 1906 |
| ATOM | 1906 | CD1 | LEU | 235 | −0.573 | −0.994 | 27.486 | 1.00 | 0.33 | 1SG | 1907 |
| ATOM | 1907 | C | LEU | 235 | −4.533 | −2.067 | 30.239 | 1.00 | 0.33 | 1SG | 1908 |
| ATOM | 1908 | O | LEU | 235 | −5.098 | −1.000 | 30.482 | 1.00 | 0.33 | 1SG | 1909 |
| ATOM | 1909 | N | PHE | 236 | −5.179 | −3.212 | 29.933 | 1.00 | 0.17 | 1SG | 1910 |
| ATOM | 1910 | CA | PHE | 236 | −6.583 | −3.250 | 29.668 | 1.00 | 0.17 | 1SG | 1911 |
| ATOM | 1911 | CB | PHE | 236 | −7.181 | −4.654 | 29.503 | 1.00 | 0.17 | 1SG | 1912 |
| ATOM | 1912 | CG | PHE | 236 | −8.538 | −4.365 | 28.986 | 1.00 | 0.17 | 1SG | 1913 |
| ATOM | 1913 | CD1 | PHE | 236 | −8.698 | −4.079 | 27.651 | 1.00 | 0.17 | 1SG | 1914 |
| ATOM | 1914 | CD2 | PHE | 236 | −9.628 | −4.329 | 29.824 | 1.00 | 0.17 | 1SG | 1915 |
| ATOM | 1915 | CE1 | PHE | 236 | −9.939 | −3.791 | 27.144 | 1.00 | 0.17 | 1SG | 1916 |
| ATOM | 1916 | CE2 | PHE | 236 | −10.873 | −4.041 | 29.321 | 1.00 | 0.17 | 1SG | 1917 |
| ATOM | 1917 | CZ | PHE | 236 | −11.032 | −3.772 | 27.979 | 1.00 | 0.17 | 1SG | 1918 |
| ATOM | 1918 | C | PHE | 236 | −7.305 | −2.580 | 30.786 | 1.00 | 0.17 | 1SG | 1919 |
| ATOM | 1919 | O | PHE | 236 | −8.289 | −1.882 | 30.550 | 1.00 | 0.17 | 1SG | 1920 |
| ATOM | 1920 | N | SER | 237 | −6.855 | −2.761 | 32.038 | 1.00 | 0.12 | 1SG | 1921 |
| ATOM | 1921 | CA | SER | 237 | −7.507 | −2.031 | 33.084 | 1.00 | 0.12 | 1SG | 1922 |
| ATOM | 1922 | CB | SER | 237 | −7.842 | −2.886 | 34.319 | 1.00 | 0.12 | 1SG | 1923 |
| ATOM | 1923 | OG | SER | 237 | −8.801 | −3.881 | 33.990 | 1.00 | 0.12 | 1SG | 1924 |
| ATOM | 1924 | C | SER | 237 | −6.558 | −0.964 | 33.536 | 1.00 | 0.12 | 1SG | 1925 |
| ATOM | 1925 | O | SER | 237 | −5.378 | −1.222 | 33.759 | 1.00 | 0.12 | 1SG | 1926 |
| ATOM | 1926 | N | GLY | 238 | −7.054 | 0.285 | 33.653 | 1.00 | 0.17 | 1SG | 1927 |
| ATOM | 1927 | CA | GLY | 238 | −6.245 | 1.372 | 34.123 | 1.00 | 0.17 | 1SG | 1928 |
| ATOM | 1928 | C | GLY | 238 | −5.914 | 1.061 | 35.542 | 1.00 | 0.17 | 1SG | 1929 |
| ATOM | 1929 | O | GLY | 238 | −4.824 | 1.361 | 36.028 | 1.00 | 0.17 | 1SG | 1930 |
| ATOM | 1930 | N | SER | 239 | −6.877 | 0.445 | 36.246 | 1.00 | 0.26 | 1SG | 1931 |
| ATOM | 1931 | CA | SER | 239 | −6.690 | 0.109 | 37.621 | 1.00 | 0.26 | 1SG | 1932 |
| ATOM | 1932 | CB | SER | 239 | −7.929 | −0.567 | 38.226 | 1.00 | 0.26 | 1SG | 1933 |
| ATOM | 1933 | OG | SER | 239 | −8.203 | −1.774 | 37.531 | 1.00 | 0.26 | 1SG | 1934 |
| ATOM | 1934 | C | SER | 239 | −5.541 | −0.846 | 37.721 | 1.00 | 0.26 | 1SG | 1935 |
| ATOM | 1935 | O | SER | 239 | −4.690 | −0.710 | 38.599 | 1.00 | 0.26 | 1SG | 1936 |
| ATOM | 1936 | N | ALA | 240 | −5.475 | −1.840 | 36.816 | 1.00 | 0.18 | 1SG | 1937 |
| ATOM | 1937 | CA | ALA | 240 | −4.411 | −2.803 | 36.869 | 1.00 | 0.18 | 1SG | 1938 |
| ATOM | 1938 | CB | ALA | 240 | −4.550 | −3.899 | 35.801 | 1.00 | 0.18 | 1SG | 1939 |
| ATOM | 1939 | C | ALA | 240 | −3.127 | −2.086 | 36.610 | 1.00 | 0.18 | 1SG | 1940 |
| ATOM | 1940 | O | ALA | 240 | −2.107 | −2.337 | 37.251 | 1.00 | 0.18 | 1SG | 1941 |
| ATOM | 1941 | N | TYR | 241 | −3.170 | −1.150 | 35.650 | 1.00 | 0.15 | 1SG | 1942 |
| ATOM | 1942 | CA | TYR | 241 | −2.038 | −0.388 | 35.217 | 1.00 | 0.15 | 1SG | 1943 |
| ATOM | 1943 | CB | TYR | 241 | −2.374 | 0.379 | 33.926 | 1.00 | 0.15 | 1SG | 1944 |
| ATOM | 1944 | CG | TYR | 241 | −1.202 | 1.177 | 33.480 | 1.00 | 0.15 | 1SG | 1945 |
| ATOM | 1945 | CD1 | TYR | 241 | −0.157 | 0.573 | 32.818 | 1.00 | 0.15 | 1SG | 1946 |
| ATOM | 1946 | CD2 | TYR | 241 | −1.170 | 2.531 | 33.700 | 1.00 | 0.15 | 1SG | 1947 |
| ATOM | 1947 | CE1 | TYR | 241 | 0.922 | 1.317 | 32.401 | 1.00 | 0.15 | 1SG | 1948 |
| ATOM | 1948 | CE2 | TYR | 241 | −0.094 | 3.276 | 33.286 | 1.00 | 0.15 | 1SG | 1949 |
| ATOM | 1949 | CZ | TYR | 241 | 0.949 | 2.668 | 32.636 | 1.00 | 0.15 | 1SG | 1950 |
| ATOM | 1950 | OH | TYR | 241 | 2.052 | 3.435 | 32.212 | 1.00 | 0.15 | 1SG | 1951 |
| ATOM | 1951 | C | TYR | 241 | −1.683 | 0.588 | 36.290 | 1.00 | 0.15 | 1SG | 1952 |
| ATOM | 1952 | O | TYR | 241 | −2.530 | 0.982 | 37.088 | 1.00 | 0.15 | 1SG | 1953 |
| ATOM | 1953 | N | PHE | 242 | −0.391 | 0.978 | 36.334 | 1.00 | 0.40 | 1SG | 1954 |
| ATOM | 1954 | CA | PHE | 242 | 0.087 | 1.915 | 37.306 | 1.00 | 0.40 | 1SG | 1955 |
| ATOM | 1955 | CB | PHE | 242 | 1.594 | 1.748 | 37.559 | 1.00 | 0.40 | 1SG | 1956 |
| ATOM | 1956 | CG | PHE | 242 | 2.016 | 2.744 | 38.577 | 1.00 | 0.40 | 1SG | 1957 |
| ATOM | 1957 | CD1 | PHE | 242 | 1.823 | 2.480 | 39.909 | 1.00 | 0.40 | 1SG | 1958 |
| ATOM | 1958 | CD2 | PHE | 242 | 2.613 | 3.927 | 38.204 | 1.00 | 0.40 | 1SG | 1959 |
| ATOM | 1959 | CE1 | PHE | 242 | 2.216 | 3.390 | 40.858 | 1.00 | 0.40 | 1SG | 1960 |
| ATOM | 1960 | CE2 | PHE | 242 | 3.006 | 4.840 | 39.150 | 1.00 | 0.40 | 1SG | 1961 |
| ATOM | 1961 | CZ | PHE | 242 | 2.806 | 4.573 | 40.482 | 1.00 | 0.40 | 1SG | 1962 |
| ATOM | 1962 | C | PHE | 242 | −0.138 | 3.272 | 36.719 | 1.00 | 0.40 | 1SG | 1963 |
| ATOM | 1963 | O | PHE | 242 | 0.762 | 3.877 | 36.137 | 1.00 | 0.40 | 1SG | 1964 |
| ATOM | 1964 | N | VAL | 243 | −1.367 | 3.787 | 36.891 | 1.00 | 0.58 | 1SG | 1965 |
| ATOM | 1965 | CA | VAL | 243 | −1.809 | 5.047 | 36.367 | 1.00 | 0.58 | 1SG | 1966 |
| ATOM | 1966 | CB | VAL | 243 | −3.320 | 5.045 | 36.281 | 1.00 | 0.58 | 1SG | 1967 |
| ATOM | 1967 | CG1 | VAL | 243 | −3.858 | 4.641 | 37.655 | 1.00 | 0.58 | 1SG | 1968 |
| ATOM | 1968 | CG2 | VAL | 243 | −3.876 | 6.372 | 35.746 | 1.00 | 0.58 | 1SG | 1969 |
| ATOM | 1969 | C | VAL | 243 | −1.273 | 6.115 | 37.274 | 1.00 | 0.58 | 1SG | 1970 |
| ATOM | 1970 | O | VAL | 243 | −0.493 | 5.815 | 38.173 | 1.00 | 0.58 | 1SG | 1971 |
| ATOM | 1971 | N | VAL | 244 | −1.618 | 7.399 | 37.029 | 1.00 | 0.42 | 1SG | 1972 |
| ATOM | 1972 | CA | VAL | 244 | −1.164 | 8.490 | 37.841 | 1.00 | 0.42 | 1SG | 1973 |
| ATOM | 1973 | CB | VAL | 244 | −1.595 | 9.851 | 37.384 | 1.00 | 0.42 | 1SG | 1974 |
| ATOM | 1974 | CG1 | VAL | 244 | −1.049 | 10.100 | 35.968 | 1.00 | 0.42 | 1SG | 1975 |
| ATOM | 1975 | CG2 | VAL | 244 | −3.118 | 9.964 | 37.541 | 1.00 | 0.42 | 1SG | 1976 |
| ATOM | 1976 | C | VAL | 244 | −1.743 | 8.296 | 39.203 | 1.00 | 0.42 | 1SG | 1977 |
| ATOM | 1977 | O | VAL | 244 | −1.120 | 8.645 | 40.205 | 1.00 | 0.42 | 1SG | 1978 |
| ATOM | 1978 | N | THR | 245 | −2.963 | 7.729 | 39.273 | 1.00 | 0.29 | 1SG | 1979 |
| ATOM | 1979 | CA | THR | 245 | −3.586 | 7.490 | 40.543 | 1.00 | 0.29 | 1SG | 1980 |
| ATOM | 1980 | CB | THR | 245 | −4.860 | 6.695 | 40.403 | 1.00 | 0.29 | 1SG | 1981 |
| ATOM | 1981 | OG1 | THR | 245 | −5.795 | 7.389 | 39.588 | 1.00 | 0.29 | 1SG | 1982 |
| ATOM | 1982 | CG2 | THR | 245 | −5.455 | 6.414 | 41.795 | 1.00 | 0.29 | 1SG | 1983 |
| ATOM | 1983 | C | THR | 245 | −2.633 | 6.639 | 41.310 | 1.00 | 0.29 | 1SG | 1984 |
| ATOM | 1984 | O | THR | 245 | −2.296 | 6.918 | 42.460 | 1.00 | 0.29 | 1SG | 1985 |
| ATOM | 1985 | N | ARG | 246 | −2.149 | 5.574 | 40.656 | 1.00 | 0.43 | 1SG | 1986 |
| ATOM | 1986 | CA | ARG | 246 | −1.167 | 4.731 | 41.254 | 1.00 | 0.43 | 1SG | 1987 |
| ATOM | 1987 | CB | ARG | 246 | −0.912 | 3.431 | 40.479 | 1.00 | 0.43 | 1SG | 1988 |
| ATOM | 1988 | CG | ARG | 246 | −1.482 | 2.243 | 41.256 | 1.00 | 0.43 | 1SG | 1989 |
| ATOM | 1989 | CD | ARG | 246 | −2.254 | 1.220 | 40.434 | 1.00 | 0.43 | 1SG | 1990 |
| ATOM | 1990 | NE | ARG | 246 | −3.630 | 1.775 | 40.353 | 1.00 | 0.43 | 1SG | 1991 |
| ATOM | 1991 | CZ | ARG | 246 | −4.448 | 1.673 | 41.441 | 1.00 | 0.43 | 1SG | 1992 |
| ATOM | 1992 | NH1 | ARG | 246 | −4.017 | 1.019 | 42.560 | 1.00 | 0.43 | 1SG | 1993 |
| ATOM | 1993 | NH2 | ARG | 246 | −5.692 | 2.238 | 41.420 | 1.00 | 0.43 | 1SG | 1994 |
| ATOM | 1994 | C | ARG | 246 | 0.094 | 5.506 | 41.421 | 1.00 | 0.43 | 1SG | 1995 |
| ATOM | 1995 | O | ARG | 246 | 0.866 | 5.252 | 42.342 | 1.00 | 0.43 | 1SG | 1996 |
| ATOM | 1996 | N | GLU | 247 | 0.333 | 6.494 | 40.549 | 1.00 | 0.52 | 1SG | 1997 |
| ATOM | 1997 | CA | GLU | 247 | 1.530 | 7.270 | 40.639 | 1.00 | 0.52 | 1SG | 1998 |
| ATOM | 1998 | CB | GLU | 247 | 1.569 | 8.400 | 39.601 | 1.00 | 0.52 | 1SG | 1999 |
| ATOM | 1999 | CG | GLU | 247 | 2.687 | 9.406 | 39.846 | 1.00 | 0.52 | 1SG | 2000 |
| ATOM | 2000 | CD | GLU | 247 | 4.024 | 8.694 | 39.729 | 1.00 | 0.52 | 1SG | 2001 |
| ATOM | 2001 | OE1 | GLU | 247 | 4.241 | 7.705 | 40.477 | 1.00 | 0.52 | 1SG | 2002 |
| ATOM | 2002 | OE2 | GLU | 247 | 4.859 | 9.145 | 38.901 | 1.00 | 0.52 | 1SG | 2003 |
| ATOM | 2003 | C | GLU | 247 | 1.569 | 7.870 | 42.001 | 1.00 | 0.52 | 1SG | 2004 |
| ATOM | 2004 | O | GLU | 247 | 2.602 | 7.839 | 42.668 | 1.00 | 0.52 | 1SG | 2005 |
| ATOM | 2005 | N | TYR | 248 | 0.427 | 8.403 | 42.461 | 1.00 | 0.52 | 1SG | 2006 |
| ATOM | 2006 | CA | TYR | 248 | 0.385 | 8.938 | 43.786 | 1.00 | 0.52 | 1SG | 2007 |
| ATOM | 2007 | CB | TYR | 248 | −0.944 | 9.622 | 44.144 | 1.00 | 0.52 | 1SG | 2008 |
| ATOM | 2008 | CG | TYR | 248 | −0.875 | 11.024 | 43.656 | 1.00 | 0.52 | 1SG | 2009 |
| ATOM | 2009 | CD1 | TYR | 248 | −1.199 | 11.391 | 42.369 | 1.00 | 0.52 | 1SG | 2010 |
| ATOM | 2010 | CD2 | TYR | 248 | −0.463 | 11.991 | 44.534 | 1.00 | 0.52 | 1SG | 2011 |
| ATOM | 2011 | CE1 | TYR | 248 | −1.112 | 12.709 | 41.979 | 1.00 | 0.52 | 1SG | 2012 |
| ATOM | 2012 | CE2 | TYR | 248 | −0.373 | 13.305 | 44.153 | 1.00 | 0.52 | 1SG | 2013 |
| ATOM | 2013 | CZ | TYR | 248 | −0.696 | 13.668 | 42.873 | 1.00 | 0.52 | 1SG | 2014 |
| ATOM | 2014 | OH | TYR | 248 | −0.602 | 15.020 | 42.487 | 1.00 | 0.52 | 1SG | 2015 |
| ATOM | 2015 | C | TYR | 248 | 0.594 | 7.848 | 44.785 | 1.00 | 0.52 | 1SG | 2016 |
| ATOM | 2016 | O | TYR | 248 | 1.271 | 8.047 | 45.792 | 1.00 | 0.52 | 1SG | 2017 |
| ATOM | 2017 | N | VAL | 249 | 0.016 | 6.660 | 44.537 | 1.00 | 0.38 | 1SG | 2018 |
| ATOM | 2018 | CA | VAL | 249 | 0.079 | 5.610 | 45.509 | 1.00 | 0.38 | 1SG | 2019 |
| ATOM | 2019 | CB | VAL | 249 | −0.754 | 4.404 | 45.147 | 1.00 | 0.38 | 1SG | 2020 |
| ATOM | 2020 | CG1 | VAL | 249 | 0.002 | 3.457 | 44.201 | 1.00 | 0.38 | 1SG | 2021 |
| ATOM | 2021 | CG2 | VAL | 249 | −1.193 | 3.727 | 46.447 | 1.00 | 0.38 | 1SG | 2022 |
| ATOM | 2022 | C | VAL | 249 | 1.501 | 5.176 | 45.670 | 1.00 | 0.38 | 1SG | 2023 |
| ATOM | 2023 | O | VAL | 249 | 1.941 | 4.878 | 46.779 | 1.00 | 0.38 | 1SG | 2024 |
| ATOM | 2024 | N | GLY | 250 | 2.270 | 5.156 | 44.562 | 1.00 | 0.18 | 1SG | 2025 |
| ATOM | 2025 | CA | GLY | 250 | 3.620 | 4.673 | 44.609 | 1.00 | 0.18 | 1SG | 2026 |
| ATOM | 2026 | C | GLY | 250 | 4.419 | 5.521 | 45.544 | 1.00 | 0.18 | 1SG | 2027 |
| ATOM | 2027 | O | GLY | 250 | 4.193 | 6.723 | 45.676 | 1.00 | 0.18 | 1SG | 2028 |
| ATOM | 2028 | N | TYR | 251 | 5.403 | 4.891 | 46.211 | 1.00 | 0.20 | 1SG | 2029 |
| ATOM | 2029 | CA | TYR | 251 | 6.227 | 5.617 | 47.121 | 1.00 | 0.20 | 1SG | 2030 |
| ATOM | 2030 | CB | TYR | 251 | 7.021 | 4.733 | 48.101 | 1.00 | 0.20 | 1SG | 2031 |
| ATOM | 2031 | CG | TYR | 251 | 7.868 | 3.770 | 47.333 | 1.00 | 0.20 | 1SG | 2032 |
| ATOM | 2032 | CD1 | TYR | 251 | 9.062 | 4.155 | 46.760 | 1.00 | 0.20 | 1SG | 2033 |
| ATOM | 2033 | CD2 | TYR | 251 | 7.463 | 2.460 | 47.209 | 1.00 | 0.20 | 1SG | 2034 |
| ATOM | 2034 | CE1 | TYR | 251 | 9.830 | 3.249 | 46.064 | 1.00 | 0.20 | 1SG | 2035 |
| ATOM | 2035 | CE2 | TYR | 251 | 8.226 | 1.551 | 46.516 | 1.00 | 0.20 | 1SG | 2036 |
| ATOM | 2036 | CZ | TYR | 251 | 9.409 | 1.945 | 45.940 | 1.00 | 0.20 | 1SG | 2037 |
| ATOM | 2037 | OH | TYR | 251 | 10.190 | 1.008 | 45.230 | 1.00 | 0.20 | 1SG | 2038 |
| ATOM | 2038 | C | TYR | 251 | 7.204 | 6.398 | 46.315 | 1.00 | 0.20 | 1SG | 2039 |
| ATOM | 2039 | O | TYR | 251 | 7.416 | 6.125 | 45.134 | 1.00 | 0.20 | 1SG | 2040 |
| ATOM | 2040 | N | VAL | 252 | 7.810 | 7.422 | 46.937 | 1.00 | 0.48 | 1SG | 2041 |
| ATOM | 2041 | CA | VAL | 252 | 8.792 | 8.178 | 46.232 | 1.00 | 0.48 | 1SG | 2042 |
| ATOM | 2042 | CB | VAL | 252 | 8.731 | 9.663 | 46.420 | 1.00 | 0.48 | 1SG | 2043 |
| ATOM | 2043 | CG1 | VAL | 252 | 7.536 | 10.219 | 45.644 | 1.00 | 0.48 | 1SG | 2044 |
| ATOM | 2044 | CG2 | VAL | 252 | 8.664 | 9.943 | 47.928 | 1.00 | 0.48 | 1SG | 2045 |
| ATOM | 2045 | C | VAL | 252 | 10.130 | 7.747 | 46.707 | 1.00 | 0.48 | 1SG | 2046 |
| ATOM | 2046 | O | VAL | 252 | 10.310 | 7.358 | 47.859 | 1.00 | 0.48 | 1SG | 2047 |
| ATOM | 2047 | N | LEU | 253 | 11.095 | 7.775 | 45.776 | 1.00 | 0.72 | 1SG | 2048 |
| ATOM | 2048 | CA | LEU | 253 | 12.455 | 7.422 | 46.060 | 1.00 | 0.72 | 1SG | 2049 |
| ATOM | 2049 | CB | LEU | 253 | 13.021 | 6.225 | 45.268 | 1.00 | 0.72 | 1SG | 2050 |
| ATOM | 2050 | CG | LEU | 253 | 12.799 | 4.834 | 45.866 | 1.00 | 0.72 | 1SG | 2051 |
| ATOM | 2051 | CD2 | LEU | 253 | 13.381 | 4.768 | 47.288 | 1.00 | 0.72 | 1SG | 2052 |
| ATOM | 2052 | CD1 | LEU | 253 | 13.416 | 3.755 | 44.965 | 1.00 | 0.72 | 1SG | 2053 |
| ATOM | 2053 | C | LEU | 253 | 13.310 | 8.552 | 45.631 | 1.00 | 0.72 | 1SG | 2054 |
| ATOM | 2054 | O | LEU | 253 | 12.906 | 9.407 | 44.847 | 1.00 | 0.72 | 1SG | 2055 |
| ATOM | 2055 | N | GLU | 254 | 14.544 | 8.565 | 46.156 | 1.00 | 0.57 | 1SG | 2056 |
| ATOM | 2056 | CA | GLU | 254 | 15.482 | 9.570 | 45.790 | 1.00 | 0.57 | 1SG | 2057 |
| ATOM | 2057 | CB | GLU | 254 | 16.819 | 9.453 | 46.543 | 1.00 | 0.57 | 1SG | 2058 |
| ATOM | 2058 | CG | GLU | 254 | 17.747 | 10.651 | 46.348 | 1.00 | 0.57 | 1SG | 2059 |
| ATOM | 2059 | CD | GLU | 254 | 17.251 | 11.777 | 47.241 | 1.00 | 0.57 | 1SG | 2060 |
| ATOM | 2060 | OE1 | GLU | 254 | 17.333 | 11.621 | 48.489 | 1.00 | 0.57 | 1SG | 2061 |
| ATOM | 2061 | OE2 | GLU | 254 | 16.781 | 12.807 | 46.688 | 1.00 | 0.57 | 1SG | 2062 |
| ATOM | 2062 | C | GLU | 254 | 15.759 | 9.377 | 44.336 | 1.00 | 0.57 | 1SG | 2063 |
| ATOM | 2063 | O | GLU | 254 | 15.736 | 8.254 | 43.834 | 1.00 | 0.57 | 1SG | 2064 |
| ATOM | 2064 | N | ASN | 255 | 16.007 | 10.492 | 43.620 | 1.00 | 0.30 | 1SG | 2065 |
| ATOM | 2065 | CA | ASN | 255 | 16.291 | 10.430 | 42.219 | 1.00 | 0.30 | 1SG | 2066 |
| ATOM | 2066 | CB | ASN | 255 | 16.552 | 11.817 | 41.598 | 1.00 | 0.30 | 1SG | 2067 |
| ATOM | 2067 | CG | ASN | 255 | 17.698 | 12.521 | 42.309 | 1.00 | 0.30 | 1SG | 2068 |
| ATOM | 2068 | OD1 | ASN | 255 | 17.786 | 12.505 | 43.536 | 1.00 | 0.30 | 1SG | 2069 |
| ATOM | 2069 | ND2 | ASN | 255 | 18.605 | 13.156 | 41.519 | 1.00 | 0.30 | 1SG | 2070 |
| ATOM | 2070 | C | ASN | 255 | 17.537 | 9.644 | 42.020 | 1.00 | 0.30 | 1SG | 2071 |
| ATOM | 2071 | O | ASN | 255 | 17.622 | 8.802 | 41.127 | 1.00 | 0.30 | 1SG | 2072 |
| ATOM | 2072 | N | GLU | 256 | 18.535 | 9.893 | 42.880 | 1.00 | 0.32 | 1SG | 2073 |
| ATOM | 2073 | CA | GLU | 256 | 19.807 | 9.259 | 42.740 | 1.00 | 0.32 | 1SG | 2074 |
| ATOM | 2074 | CB | GLU | 256 | 20.817 | 9.710 | 43.807 | 1.00 | 0.32 | 1SG | 2075 |
| ATOM | 2075 | CG | GLU | 256 | 22.235 | 9.205 | 43.544 | 1.00 | 0.32 | 1SG | 2076 |
| ATOM | 2076 | CD | GLU | 256 | 22.770 | 9.959 | 42.335 | 1.00 | 0.32 | 1SG | 2077 |
| ATOM | 2077 | OE1 | GLU | 256 | 22.025 | 10.824 | 41.802 | 1.00 | 0.32 | 1SG | 2078 |
| ATOM | 2078 | OE2 | GLU | 256 | 23.929 | 9.680 | 41.926 | 1.00 | 0.32 | 1SG | 2079 |
| ATOM | 2079 | C | GLU | 256 | 19.605 | 7.792 | 42.895 | 1.00 | 0.32 | 1SG | 2080 |
| ATOM | 2080 | O | GLU | 256 | 20.233 | 6.988 | 42.207 | 1.00 | 0.32 | 1SG | 2081 |
| ATOM | 2081 | N | ASN | 257 | 18.692 | 7.411 | 43.801 | 1.00 | 0.40 | 1SG | 2082 |
| ATOM | 2082 | CA | ASN | 257 | 18.471 | 6.026 | 44.082 | 1.00 | 0.40 | 1SG | 2083 |
| ATOM | 2083 | CB | ASN | 257 | 17.303 | 5.842 | 45.068 | 1.00 | 0.40 | 1SG | 2084 |
| ATOM | 2084 | CG | ASN | 257 | 17.420 | 4.465 | 45.677 | 1.00 | 0.40 | 1SG | 2085 |
| ATOM | 2085 | OD1 | ASN | 257 | 16.438 | 3.774 | 45.939 | 1.00 | 0.40 | 1SG | 2086 |
| ATOM | 2086 | ND2 | ASN | 257 | 18.690 | 4.061 | 45.928 | 1.00 | 0.40 | 1SG | 2087 |
| ATOM | 2087 | C | ASN | 257 | 18.089 | 5.346 | 42.807 | 1.00 | 0.40 | 1SG | 2088 |
| ATOM | 2088 | O | ASN | 257 | 18.667 | 4.324 | 42.438 | 1.00 | 0.40 | 1SG | 2089 |
| ATOM | 2089 | N | ILE | 258 | 17.104 | 5.911 | 42.087 | 1.00 | 0.45 | 1SG | 2090 |
| ATOM | 2090 | CA | ILE | 258 | 16.661 | 5.296 | 40.872 | 1.00 | 0.45 | 1SG | 2091 |
| ATOM | 2091 | CB | ILE | 258 | 15.361 | 5.859 | 40.361 | 1.00 | 0.45 | 1SG | 2092 |
| ATOM | 2092 | CG2 | ILE | 258 | 14.310 | 5.630 | 41.460 | 1.00 | 0.45 | 1SG | 2093 |
| ATOM | 2093 | CG1 | ILE | 258 | 15.482 | 7.330 | 39.937 | 1.00 | 0.45 | 1SG | 2094 |
| ATOM | 2094 | CD1 | ILE | 258 | 16.077 | 7.498 | 38.542 | 1.00 | 0.45 | 1SG | 2095 |
| ATOM | 2095 | C | ILE | 258 | 17.738 | 5.449 | 39.848 | 1.00 | 0.45 | 1SG | 2096 |
| ATOM | 2096 | O | ILE | 258 | 17.936 | 4.578 | 39.002 | 1.00 | 0.45 | 1SG | 2097 |
| ATOM | 2097 | N | GLN | 259 | 18.482 | 6.567 | 39.929 | 1.00 | 0.67 | 1SG | 2098 |
| ATOM | 2098 | CA | GLN | 259 | 19.499 | 6.889 | 38.973 | 1.00 | 0.67 | 1SG | 2099 |
| ATOM | 2099 | CB | GLN | 259 | 20.229 | 8.203 | 39.311 | 1.00 | 0.67 | 1SG | 2100 |
| ATOM | 2100 | CG | GLN | 259 | 21.328 | 8.586 | 38.315 | 1.00 | 0.67 | 1SG | 2101 |
| ATOM | 2101 | CD | GLN | 259 | 22.607 | 7.855 | 38.701 | 1.00 | 0.67 | 1SG | 2102 |
| ATOM | 2102 | OE1 | GLN | 259 | 23.529 | 7.728 | 37.898 | 1.00 | 0.67 | 1SG | 2103 |
| ATOM | 2103 | NE2 | GLN | 259 | 22.672 | 7.369 | 39.970 | 1.00 | 0.67 | 1SG | 2104 |
| ATOM | 2104 | C | GLN | 259 | 20.508 | 5.793 | 38.942 | 1.00 | 0.67 | 1SG | 2105 |
| ATOM | 2105 | O | GLN | 259 | 20.971 | 5.407 | 37.870 | 1.00 | 0.67 | 1SG | 2106 |
| ATOM | 2106 | N | LYS | 260 | 20.874 | 5.228 | 40.105 | 1.00 | 0.99 | 1SG | 2107 |
| ATOM | 2107 | CA | LYS | 260 | 21.880 | 4.215 | 40.006 | 1.00 | 0.99 | 1SG | 2108 |
| ATOM | 2108 | CB | LYS | 260 | 22.636 | 3.893 | 41.302 | 1.00 | 0.99 | 1SG | 2109 |
| ATOM | 2109 | CG | LYS | 260 | 21.860 | 3.165 | 42.387 | 1.00 | 0.99 | 1SG | 2110 |
| ATOM | 2110 | CD | LYS | 260 | 22.825 | 2.562 | 43.402 | 1.00 | 0.99 | 1SG | 2111 |
| ATOM | 2111 | CE | LYS | 260 | 23.628 | 1.393 | 42.832 | 1.00 | 0.99 | 1SG | 2112 |
| ATOM | 2112 | NZ | LYS | 260 | 22.761 | 0.201 | 42.699 | 1.00 | 0.99 | 1SG | 2113 |
| ATOM | 2113 | C | LYS | 260 | 21.268 | 2.987 | 39.424 | 1.00 | 0.99 | 1SG | 2114 |
| ATOM | 2114 | O | LYS | 260 | 20.320 | 3.076 | 38.646 | 1.00 | 0.99 | 1SG | 2115 |
| ATOM | 2115 | N | LEU | 261 | 21.836 | 1.804 | 39.731 | 1.00 | 0.94 | 1SG | 2116 |
| ATOM | 2116 | CA | LEU | 261 | 21.323 | 0.613 | 39.121 | 1.00 | 0.94 | 1SG | 2117 |
| ATOM | 2117 | CB | LEU | 261 | 22.257 | −0.603 | 39.245 | 1.00 | 0.94 | 1SG | 2118 |
| ATOM | 2118 | CG | LEU | 261 | 23.615 | −0.413 | 38.553 | 1.00 | 0.94 | 1SG | 2119 |
| ATOM | 2119 | CD2 | LEU | 261 | 24.399 | 0.738 | 39.198 | 1.00 | 0.94 | 1SG | 2120 |
| ATOM | 2120 | CD1 | LEU | 261 | 23.458 | −0.263 | 37.032 | 1.00 | 0.94 | 1SG | 2121 |
| ATOM | 2121 | C | LEU | 261 | 20.059 | 0.232 | 39.806 | 1.00 | 0.94 | 1SG | 2122 |
| ATOM | 2122 | O | LEU | 261 | 20.019 | −0.777 | 40.510 | 1.00 | 0.94 | 1SG | 2123 |
| ATOM | 2123 | N | MET | 262 | 19.000 | 1.033 | 39.578 | 1.00 | 0.49 | 1SG | 2124 |
| ATOM | 2124 | CA | MET | 262 | 17.680 | 0.813 | 40.089 | 1.00 | 0.49 | 1SG | 2125 |
| ATOM | 2125 | CB | MET | 262 | 16.916 | −0.293 | 39.340 | 1.00 | 0.49 | 1SG | 2126 |
| ATOM | 2126 | CG | MET | 262 | 16.629 | 0.001 | 37.868 | 1.00 | 0.49 | 1SG | 2127 |
| ATOM | 2127 | SD | MET | 262 | 15.727 | −1.334 | 37.022 | 1.00 | 0.49 | 1SG | 2128 |
| ATOM | 2128 | CE | MET | 262 | 15.701 | −0.528 | 35.396 | 1.00 | 0.49 | 1SG | 2129 |
| ATOM | 2129 | C | MET | 262 | 17.764 | 0.372 | 41.510 | 1.00 | 0.49 | 1SG | 2130 |
| ATOM | 2130 | O | MET | 262 | 17.274 | −0.707 | 41.835 | 1.00 | 0.49 | 1SG | 2131 |
| ATOM | 2131 | N | GLU | 263 | 18.389 | 1.165 | 42.403 | −1.00 | 0.27 | 1SG | 2132 |
| ATOM | 2132 | CA | GLU | 263 | 18.418 | 0.669 | 43.746 | 1.00 | 0.27 | 1SG | 2133 |
| ATOM | 2133 | CB | GLU | 263 | 19.205 | 1.491 | 44.777 | 1.00 | 0.27 | 1SG | 2134 |
| ATOM | 2134 | CG | GLU | 263 | 20.688 | 1.148 | 44.836 | 1.00 | 0.27 | 1SG | 2135 |
| ATOM | 2135 | CD | GLU | 263 | 21.318 | 1.975 | 45.948 | 1.00 | 0.27 | 1SG | 2136 |
| ATOM | 2136 | OE1 | GLU | 263 | 21.214 | 3.228 | 45.891 | 1.00 | 0.27 | 1SG | 2137 |
| ATOM | 2137 | OE2 | GLU | 263 | 21.908 | 1.361 | 46.877 | 1.00 | 0.27 | 1SG | 2138 |
| ATOM | 2138 | C | GLU | 26.3 | 17.017 | 0.590 | 44.230 | 1.00 | 0.27 | 1SG | 2139 |
| ATOM | 2139 | O | GLU | 263 | 16.144 | 1.309 | 43.746 | 1.00 | 0.27 | 1SG | 2140 |
| ATOM | 2140 | N | TRP | 264 | 16.785 | −0.314 | 45.201 | 1.00 | 0.25 | 1SG | 2141 |
| ATOM | 2141 | CA | TRP | 264 | 15.470 | −0.550 | 45.717 | 1.00 | 0.25 | 1SG | 2142 |
| ATOM | 2142 | CB | TRP | 264 | 14.950 | 0.550 | 46.660 | 1.00 | 0.25 | 1SG | 2143 |
| ATOM | 2143 | CG | TRP | 264 | 15.592 | 0.509 | 48.029 | 1.00 | 0.25 | 1SG | 2144 |
| ATOM | 2144 | CD2 | TRP | 264 | 16.800 | 1.195 | 48.396 | 1.00 | 0.25 | 1SG | 2145 |
| ATOM | 2145 | CD1 | TRP | 264 | 15.180 | −0.166 | 49.140 | 1.00 | 0.25 | 1SG | 2146 |
| ATOM | 2146 | NE1 | TRP | 264 | 16.059 | 0.048 | 50.174 | 1.00 | 0.25 | 1SG | 2147 |
| ATOM | 2147 | CE2 | TRP | 264 | 17.061 | 0.885 | 49.732 | 1.00 | 0.25 | 1SG | 2148 |
| ATOM | 2148 | CE3 | TRP | 264 | 17.626 | 2.014 | 47.682 | 1.00 | 0.25 | 1SG | 2149 |
| ATOM | 2149 | CZ2 | TRP | 264 | 18.154 | 1.390 | 50.377 | 1.00 | 0.25 | 1SG | 2150 |
| ATOM | 2150 | CZ3 | TRP | 264 | 18.727 | 2.520 | 48.339 | 1.00 | 0.25 | 1SG | 2151 |
| ATOM | 2151 | CH2 | TRP | 264 | 18.988 | 2.215 | 49.658 | 1.00 | 0.25 | 1SG | 2152 |
| ATOM | 2152 | C | TRP | 264 | 14.528 | −0.737 | 44.569 | 1.00 | 0.25 | 1SG | 2153 |
| ATOM | 2153 | O | TRP | 264 | 13.553 | −0.011 | 44.399 | 1.00 | 0.25 | 1SG | 2154 |
| ATOM | 2154 | N | ALA | 265 | 14.818 | −1.775 | 43.775 | 1.00 | 0.35 | 1SG | 2155 |
| ATOM | 2155 | CA | ALA | 265 | 14.170 | −2.213 | 42.573 | 1.00 | 0.35 | 1SG | 2156 |
| ATOM | 2156 | CB | ALA | 265 | 14.966 | −3.299 | 41.830 | 1.00 | 0.35 | 1SG | 2157 |
| ATOM | 2157 | C | ALA | 265 | 12.826 | −2.788 | 42.889 | 1.00 | 0.35 | 1SG | 2158 |
| ATOM | 2158 | O | ALA | 265 | 12.186 | −3.356 | 42.006 | 1.00 | 0.35 | 1SG | 2159 |
| ATOM | 2159 | N | GLN | 266 | 12.398 | −2.720 | 44.167 | 1.00 | 0.50 | 1SG | 2160 |
| ATOM | 2160 | CA | GLN | 266 | 11.230 | −3.422 | 44.627 | 1.00 | 0.50 | 1SG | 2161 |
| ATOM | 2161 | CB | GLN | 266 | 10.734 | −2.957 | 46.009 | 1.00 | 0.50 | 1SG | 2162 |
| ATOM | 2162 | CG | GLN | 266 | 11.710 | −3.212 | 47.157 | 1.00 | 0.50 | 1SG | 2163 |
| ATOM | 2163 | CD | GLN | 266 | 12.785 | −2.143 | 47.091 | 1.00 | 0.50 | 1SG | 2164 |
| ATOM | 2164 | OE1 | GLN | 266 | 12.507 | −0.951 | 46.966 | 1.00 | 0.50 | 1SG | 2165 |
| ATOM | 2165 | NE2 | GLN | 266 | 14.066 | −2.583 | 47.171 | 1.00 | 0.50 | 1SG | 2166 |
| ATOM | 2166 | C | GLN | 266 | 10.054 | −3.254 | 43.714 | 1.00 | 0.50 | 1SG | 2167 |
| ATOM | 2167 | O | GLN | 266 | 9.481 | −4.256 | 43.288 | 1.00 | 0.50 | 1SG | 2168 |
| ATOM | 2168 | N | ASP | 267 | 9.651 | −2.023 | 43.351 | 1.00 | 0.42 | 1SG | 2169 |
| ATOM | 2169 | CA | ASP | 267 | 8.468 | −1.986 | 42.537 | 1.00 | 0.42 | 1SG | 2170 |
| ATOM | 2170 | CB | ASP | 267 | 7.169 | −2.092 | 43.358 | 1.00 | 0.42 | 1SG | 2171 |
| ATOM | 2171 | CG | ASP | 267 | 6.034 | −2.521 | 42.433 | 1.00 | 0.42 | 1SG | 2172 |
| ATOM | 2172 | OD1 | ASP | 267 | 6.330 | −3.082 | 41.344 | 1.00 | 0.42 | 1SG | 2173 |
| ATOM | 2173 | OD2 | ASP | 267 | 4.855 | −2.295 | 42.811 | 1.00 | 0.42 | 1SG | 2174 |
| ATOM | 2174 | C | ASP | 267 | 8.441 | −0.688 | 41.791 | 1.00 | 0.42 | 1SG | 2175 |
| ATOM | 2175 | O | ASP | 267 | 9.467 | −0.223 | 41.300 | 1.00 | 0.42 | 1SG | 2176 |
| ATOM | 2176 | N | THR | 268 | 7.235 | −0.096 | 41.645 | 1.00 | 0.26 | 1SG | 2177 |
| ATOM | 2177 | CA | THR | 268 | 7.081 | 1.142 | 40.942 | 1.00 | 0.26 | 1SG | 2178 |
| ATOM | 2178 | CB | THR | 268 | 5.651 | 1.483 | 40.638 | 1.00 | 0.26 | 1SG | 2179 |
| ATOM | 2179 | OG1 | THR | 268 | 5.062 | 0.459 | 39.849 | 1.00 | 0.26 | 1SG | 2180 |
| ATOM | 2180 | CG2 | THR | 268 | 5.616 | 2.815 | 39.870 | 1.00 | 0.26 | 1SG | 2181 |
| ATOM | 2181 | C | THR | 268 | 7.643 | 2.227 | 41.799 | 1.00 | 0.26 | 1SG | 2182 |
| ATOM | 2182 | O | THR | 268 | 7.713 | 2.101 | 43.021 | 1.00 | 0.26 | 1SG | 2183 |
| ATOM | 2183 | N | TYR | 269 | 8.083 | 3.325 | 41.156 | 1.00 | 0.20 | 1SG | 2184 |
| ATOM | 2184 | CA | TYR | 269 | 8.705 | 4.404 | 41.856 | 1.00 | 0.20 | 1SG | 2185 |
| ATOM | 2185 | CB | TYR | 269 | 10.238 | 4.287 | 41.796 | 1.00 | 0.20 | 1SG | 2186 |
| ATOM | 2186 | CG | TYR | 269 | 10.879 | 5.621 | 41.871 | 1.00 | 0.20 | 1SG | 2187 |
| ATOM | 2187 | CD1 | TYR | 269 | 11.060 | 6.315 | 43.043 | 1.00 | 0.20 | 1SG | 2188 |
| ATOM | 2188 | CD2 | TYR | 269 | 11.300 | 6.184 | 40.697 | 1.00 | 0.20 | 1SG | 2189 |
| ATOM | 2189 | CE1 | TYR | 269 | 11.667 | 7.550 | 43.020 | 1.00 | 0.20 | 1SG | 2190 |
| ATOM | 2190 | CE2 | TYR | 269 | 11.904 | 7.411 | 40.675 | 1.00 | 0.20 | 1SG | 2191 |
| ATOM | 2191 | CZ | TYR | 269 | 12.093 | 8.098 | 41.841 | 1.00 | 0.20 | 1SG | 2192 |
| ATOM | 2192 | OH | TYR | 269 | 12.717 | 9.361 | 41.823 | 1.00 | 0.20 | 1SG | 2193 |
| ATOM | 2193 | C | TYR | 269 | 8.228 | 5.699 | 41.284 | 1.00 | 0.20 | 1SG | 2194 |
| ATOM | 2194 | O | TYR | 269 | 7.906 | 5.791 | 40.101 | 1.00 | 0.20 | 1SG | 2195 |
| ATOM | 2195 | N | SER | 270 | 8.123 | 6.734 | 42.143 | 1.00 | 0.36 | 1SG | 2196 |
| ATOM | 2196 | CA | SER | 270 | 7.684 | 8.014 | 41.676 | 1.00 | 0.36 | 1SG | 2197 |
| ATOM | 2197 | CB | SER | 270 | 6.346 | 8.460 | 42.290 | 1.00 | 0.36 | 1SG | 2198 |
| ATOM | 2198 | OG | SER | 270 | 6.459 | 8.551 | 43.701 | 1.00 | 0.36 | 1SG | 2199 |
| ATOM | 2199 | C | SER | 270 | 8.725 | 9.015 | 42.066 | 1.00 | 0.36 | 1SG | 2200 |
| ATOM | 2200 | O | SER | 270 | 9.122 | 9.096 | 43.228 | 1.00 | 0.36 | 1SG | 2201 |
| ATOM | 2201 | N | PRO | 271 | 9.215 | 9.747 | 41.102 | 1.00 | 0.61 | 1SG | 2202 |
| ATOM | 2202 | CA | PRO | 271 | 10.203 | 10.752 | 41.397 | 1.00 | 0.61 | 1SG | 2203 |
| ATOM | 2203 | CD | PRO | 271 | 9.383 | 9.190 | 39.770 | 1.00 | 0.61 | 1SG | 2204 |
| ATOM | 2204 | CB | PRO | 271 | 10.911 | 11.053 | 40.079 | 1.00 | 0.61 | 1SG | 2205 |
| ATOM | 2205 | CG | PRO | 271 | 10.701 | 9.781 | 39.241 | 1.00 | 0.61 | 1SG | 2206 |
| ATOM | 2206 | C | PRO | 271 | 9.594 | 11.991 | 41.997 | 1.00 | 0.61 | 1SG | 2207 |
| ATOM | 2207 | O | PRO | 271 | 8.908 | 12.708 | 41.273 | 1.00 | 0.61 | 1SG | 2208 |
| ATOM | 2208 | N | ASP | 272 | 9.820 | 12.231 | 43.306 | 1.00 | 0.68 | 1SG | 2209 |
| ATOM | 2209 | CA | ASP | 272 | 9.438 | 13.357 | 44.125 | 1.00 | 0.68 | 1SG | 2210 |
| ATOM | 2210 | CB | ASP | 272 | 8.961 | 12.967 | 45.533 | 1.00 | 0.68 | 1SG | 2211 |
| ATOM | 2211 | CG | ASP | 272 | 8.156 | 14.138 | 46.093 | 1.00 | 0.68 | 1SG | 2212 |
| ATOM | 2212 | OD1 | ASP | 272 | 7.935 | 15.127 | 45.341 | 1.00 | 0.68 | 1SG | 2213 |
| ATOM | 2213 | OD2 | ASP | 272 | 7.747 | 14.054 | 47.282 | 1.00 | 0.68 | 1SG | 2214 |
| ATOM | 2214 | C | ASP | 272 | 10.552 | 14.346 | 44.280 | 1.00 | 0.68 | 1SG | 2215 |
| ATOM | 2215 | O | ASP | 272 | 10.451 | 15.256 | 45.097 | 1.00 | 0.68 | 1SG | 2216 |
| ATOM | 2216 | N | GLU | 273 | 11.674 | 14.152 | 43.569 | 1.00 | 0.72 | 1SG | 2217 |
| ATOM | 2217 | CA | GLU | 273 | 12.932 | 14.848 | 43.702 | 1.00 | 0.72 | 1SG | 2218 |
| ATOM | 2218 | CB | GLU | 273 | 14.025 | 14.334 | 42.783 | 1.00 | 0.72 | 1SG | 2219 |
| ATOM | 2219 | CG | GLU | 273 | 14.331 | 12.898 | 43.133 | 1.00 | 0.72 | 1SG | 2220 |
| ATOM | 2220 | CD | GLU | 273 | 13.323 | 12.063 | 42.374 | 1.00 | 0.72 | 1SG | 2221 |
| ATOM | 2221 | OE1 | GLU | 273 | 13.500 | 11.882 | 41.139 | 1.00 | 0.72 | 1SG | 2222 |
| ATOM | 2222 | OE2 | GLU | 273 | 12.348 | 11.608 | 43.025 | 1.00 | 0.72 | 1SG | 2223 |
| ATOM | 2223 | C | GLU | 273 | 12.818 | 16.317 | 43.461 | 1.00 | 0.72 | 1SG | 2224 |
| ATOM | 2224 | O | GLU | 273 | 13.826 | 17.003 | 43.305 | 1.00 | 0.72 | 1SG | 2225 |
| ATOM | 2225 | N | PHE | 274 | 11.579 | 16.776 | 43.303 | 1.00 | 0.66 | 1SG | 2226 |
| ATOM | 2226 | CA | PHE | 274 | 11.067 | 18.093 | 43.120 | 1.00 | 0.66 | 1SG | 2227 |
| ATOM | 2227 | CB | PHE | 274 | 9.607 | 18.029 | 42.656 | 1.00 | 0.66 | 1SG | 2228 |
| ATOM | 2228 | CG | PHE | 274 | 9.257 | 19.324 | 42.024 | 1.00 | 0.66 | 1SG | 2229 |
| ATOM | 2229 | CD1 | PHE | 274 | 10.121 | 19.916 | 41.134 | 1.00 | 0.66 | 1SG | 2230 |
| ATOM | 2230 | CD2 | PHE | 274 | 8.039 | 19.906 | 42.256 | 1.00 | 0.66 | 1SG | 2231 |
| ATOM | 2231 | CE1 | PHE | 274 | 9.801 | 21.103 | 40.522 | 1.00 | 0.66 | 1SG | 2232 |
| ATOM | 2232 | CE2 | PHE | 274 | 7.717 | 21.089 | 41.642 | 1.00 | 0.66 | 1SG | 2233 |
| ATOM | 2233 | CZ | PHE | 274 | 8.595 | 21.696 | 40.778 | 1.00 | 0.66 | 1SG | 2234 |
| ATOM | 2234 | C | PHE | 274 | 11.209 | 18.811 | 44.441 | 1.00 | 0.66 | 1SG | 2235 |
| ATOM | 2235 | O | PHE | 274 | 10.435 | 19.712 | 44.749 | 1.00 | 0.66 | 1SG | 2236 |
| ATOM | 2236 | N | LEU | 275 | 12.134 | 18.337 | 45.309 | 1.00 | 0.55 | 1SG | 2237 |
| ATOM | 2237 | CA | LEU | 275 | 12.300 | 18.742 | 46.685 | 1.00 | 0.55 | 1SG | 2238 |
| ATOM | 2238 | CB | LEU | 275 | 13.623 | 18.289 | 47.333 | 1.00 | 0.55 | 1SG | 2239 |
| ATOM | 2239 | CG | LEU | 275 | 13.851 | 16.770 | 47.402 | 1.00 | 0.55 | 1SG | 2240 |
| ATOM | 2240 | CD2 | LEU | 275 | 14.223 | 16.189 | 46.035 | 1.00 | 0.55 | 1SG | 2241 |
| ATOM | 2241 | CD1 | LEU | 275 | 12.690 | 16.051 | 48.107 | 1.00 | 0.55 | 1SG | 2242 |
| ATOM | 2242 | C | LEU | 275 | 12.295 | 20.228 | 46.879 | 1.00 | 0.55 | 1SG | 2243 |
| ATOM | 2243 | O | LEU | 275 | 11.566 | 20.714 | 47.743 | 1.00 | 0.55 | 1SG | 2244 |
| ATOM | 2244 | N | TRP | 276 | 13.067 | 21.021 | 46.109 | 1.00 | 0.38 | 1SG | 2245 |
| ATOM | 2245 | CA | TRP | 276 | 13.015 | 22.444 | 46.336 | 1.00 | 0.38 | 1SG | 2246 |
| ATOM | 2246 | CB | TRP | 276 | 14.011 | 23.242 | 45.463 | 1.00 | 0.38 | 1SG | 2247 |
| ATOM | 2247 | CG | TRP | 276 | 14.053 | 24.738 | 45.671 | 1.00 | 0.38 | 1SG | 2248 |
| ATOM | 2248 | CD2 | TRP | 276 | 14.471 | 25.624 | 44.561 | 1.00 | 0.38 | 1SG | 2249 |
| ATOM | 2249 | CD1 | TRP | 276 | 13.736 | 25.427 | 46.815 | 1.00 | 0.38 | 1SG | 2250 |
| ATOM | 2250 | NE1 | TRP | 276 | 13.907 | 26.720 | 46.576 | 1.00 | 0.38 | 1SG | 2251 |
| ATOM | 2251 | CE2 | TRP | 276 | 14.318 | 26.703 | 45.297 | 1.00 | 0.38 | 1SG | 2252 |
| ATOM | 2252 | CE3 | TRP | 276 | 14.707 | 27.042 | 44.067 | 1.00 | 0.38 | 1SG | 2253 |
| ATOM | 2253 | CZ2 | TRP | 276 | 14.652 | 27.457 | 44.347 | 1.00 | 0.38 | 1SG | 2254 |
| ATOM | 2254 | CZ3 | TRP | 276 | 15.161 | 27.406 | 42.752 | 1.00 | 0.38 | 1SG | 2255 |
| ATOM | 2255 | CH2 | TRP | 276 | 14.924 | 26.377 | 43.387 | 1.00 | 0.38 | 1SG | 2256 |
| ATOM | 2256 | C | TRP | 276 | 11.615 | 22.874 | 46.011 | 1.00 | 0.38 | 1SG | 2257 |
| ATOM | 2257 | O | TRP | 276 | 10.795 | 22.069 | 45.573 | 1.00 | 0.38 | 1SG | 2258 |
| ATOM | 2258 | N | ALA | 277 | 11.282 | 24.158 | 46.242 | 1.00 | 0.23 | 1SG | 2259 |
| ATOM | 2259 | CA | ALA | 277 | 9.945 | 24.589 | 45.961 | 1.00 | 0.23 | 1SG | 2260 |
| ATOM | 2260 | CB | ALA | 277 | 9.717 | 26.091 | 46.201 | 1.00 | 0.23 | 1SG | 2261 |
| ATOM | 2261 | C | ALA | 277 | 9.704 | 24.315 | 44.517 | 1.00 | 0.23 | 1SG | 2262 |
| ATOM | 2262 | O | ALA | 277 | 10.659 | 24.260 | 43.741 | 1.00 | 0.23 | 1SG | 2263 |
| ATOM | 2263 | N | THR | 278 | 8.425 | 24.057 | 44.159 | 1.00 | 0.46 | 1SG | 2264 |
| ATOM | 2264 | CA | THR | 278 | 8.074 | 23.773 | 42.800 | 1.00 | 0.46 | 1SG | 2265 |
| ATOM | 2265 | CB | THR | 278 | 6.597 | 23.756 | 42.549 | 1.00 | 0.46 | 1SG | 2266 |
| ATOM | 2266 | OG1 | THR | 278 | 6.001 | 22.692 | 43.271 | 1.00 | 0.46 | 1SG | 2267 |
| ATOM | 2267 | CG2 | THR | 278 | 6.327 | 23.614 | 41.042 | 1.00 | 0.46 | 1SG | 2268 |
| ATOM | 2268 | C | THR | 278 | 8.659 | 24.867 | 42.005 | 1.00 | 0.46 | 1SG | 2269 |
| ATOM | 2269 | O | THR | 278 | 8.231 | 26.018 | 42.047 | 1.00 | 0.46 | 1SG | 2270 |
| ATOM | 2270 | N | ILE | 279 | 9.698 | 24.493 | 41.263 | 1.00 | 0.71 | 1SG | 2271 |
| ATOM | 2271 | CA | ILE | 279 | 10.490 | 25.450 | 40.588 | 1.00 | 0.71 | 1SG | 2272 |
| ATOM | 2272 | CB | ILE | 279 | 11.700 | 25.655 | 41.459 | 1.00 | 0.71 | 1SG | 2273 |
| ATOM | 2273 | CG2 | ILE | 279 | 12.466 | 24.329 | 41.503 | 1.00 | 0.71 | 1SG | 2274 |
| ATOM | 2274 | CD1 | ILE | 279 | 12.543 | 26.852 | 41.074 | 1.00 | 0.71 | 1SG | 2275 |
| ATOM | 2275 | CD1 | ILE | 279 | 13.273 | 26.616 | 39.786 | 1.00 | 0.71 | 1SG | 2276 |
| ATOM | 2276 | C | ILE | 279 | 10.774 | 24.798 | 39.281 | 1.00 | 0.71 | 1SG | 2277 |
| ATOM | 2277 | O | ILE | 279 | 10.537 | 23.603 | 39.158 | 1.00 | 0.71 | 1SG | 2278 |
| ATOM | 2278 | N | GLN | 280 | 11.162 | 25.555 | 38.237 | 1.00 | 0.49 | 1SG | 2279 |
| ATOM | 2279 | CA | GLN | 280 | 11.444 | 24.911 | 36.985 | 1.00 | 0.49 | 1SG | 2280 |
| ATOM | 2280 | CB | GLN | 280 | 11.457 | 25.861 | 35.776 | 1.00 | 0.49 | 1SG | 2281 |
| ATOM | 2281 | CG | GLN | 280 | 10.062 | 26.392 | 35.438 | 1.00 | 0.49 | 1SG | 2282 |
| ATOM | 2282 | CD | GLN | 280 | 10.101 | 26.998 | 34.043 | 1.00 | 0.49 | 1SG | 2283 |
| ATOM | 2283 | OE1 | GLN | 280 | 10.733 | 26.454 | 33.139 | 1.00 | 0.49 | 1SG | 2284 |
| ATOM | 2284 | NE2 | GLN | 280 | 9.405 | 28.152 | 33.859 | 1.00 | 0.49 | 1SG | 2285 |
| ATOM | 2285 | C | GLN | 280 | 12.752 | 24.180 | 37.041 | 1.00 | 0.49 | 1SG | 2286 |
| ATOM | 2286 | O | GLN | 280 | 12.895 | 23.112 | 36.453 | 1.00 | 0.49 | 1SG | 2287 |
| ATOM | 2287 | N | ARG | 281 | 13.750 | 24.734 | 37.754 | 1.00 | 0.36 | 1SG | 2288 |
| ATOM | 2288 | CA | ARG | 281 | 15.068 | 24.165 | 37.769 | 1.00 | 0.36 | 1SG | 2289 |
| ATOM | 2289 | CB | ARG | 281 | 16.086 | 24.971 | 38.594 | 1.00 | 0.36 | 1SG | 2290 |
| ATOM | 2290 | CG | ARG | 281 | 15.755 | 25.054 | 40.085 | 1.00 | 0.36 | 1SG | 2291 |
| ATOM | 2291 | CD | ARG | 281 | 16.842 | 25.748 | 40.908 | 1.00 | 0.36 | 1SG | 2292 |
| ATOM | 2292 | NE | ARG | 281 | 18.055 | 24.884 | 40.855 | 1.00 | 0.36 | 1SG | 2293 |
| ATOM | 2293 | CZ | ARG | 281 | 19.281 | 25.406 | 41.151 | 1.00 | 0.36 | 1SG | 2294 |
| ATOM | 2294 | NH1 | ARG | 281 | 19.398 | 26.721 | 41.497 | 1.00 | 0.36 | 1SG | 2295 |
| ATOM | 2295 | NH2 | ARG | 281 | 20.392 | 24.613 | 41.100 | 1.00 | 0.36 | 1SG | 2296 |
| ATOM | 2296 | C | ARG | 281 | 15.056 | 22.787 | 38.346 | 1.00 | 0.36 | 1SG | 2297 |
| ATOM | 2297 | O | ARG | 281 | 15.665 | 21.880 | 37.782 | 1.00 | 0.36 | 1SG | 2298 |
| ATOM | 2298 | N | ILE | 282 | 14.353 | 22.577 | 39.473 | 1.00 | 0.39 | 1SG | 2299 |
| ATOM | 2299 | CA | ILE | 282 | 14.382 | 21.280 | 40.097 | 1.00 | 0.39 | 1SG | 2300 |
| ATOM | 2300 | CB | ILE | 282 | 13.364 | 21.112 | 41.182 | 1.00 | 0.39 | 1SG | 2301 |
| ATOM | 2301 | CG2 | ILE | 282 | 13.147 | 19.610 | 41.370 | 1.00 | 0.39 | 1SG | 2302 |
| ATOM | 2302 | CG1 | ILE | 282 | 13.698 | 21.839 | 42.479 | 1.00 | 0.39 | 1SG | 2303 |
| ATOM | 2303 | CD1 | ILE | 282 | 12.511 | 21.747 | 43.435 | 1.00 | 0.39 | 1SG | 2304 |
| ATOM | 2304 | C | ILE | 282 | 13.993 | 20.190 | 39.153 | 1.00 | 0.39 | 1SG | 2305 |
| ATOM | 2305 | O | ILE | 282 | 14.838 | 19.361 | 38.836 | 1.00 | 0.39 | 1SG | 2306 |
| ATOM | 2306 | N | PRO | 283 | 12.780 | 20.151 | 38.662 | 1.00 | 0.35 | 1SG | 2307 |
| ATOM | 2307 | CA | PRO | 283 | 12.326 | 19.056 | 37.861 | 1.00 | 0.35 | 1SG | 2308 |
| ATOM | 2308 | CD | PRO | 283 | 11.977 | 21.328 | 38.433 | 1.00 | 0.35 | 1SG | 2309 |
| ATOM | 2309 | CB | PRO | 283 | 10.904 | 19.413 | 37.438 | 1.00 | 0.35 | 1SG | 2310 |
| ATOM | 2310 | CG | PRO | 283 | 10.957 | 20.945 | 37.351 | 1.00 | 0.35 | 1SG | 2311 |
| ATOM | 2311 | C | PRO | 283 | 13.221 | 18.925 | 36.681 | 1.00 | 0.35 | 1SG | 2312 |
| ATOM | 2312 | O | PRO | 283 | 13.335 | 17.822 | 36.155 | 1.00 | 0.35 | 1SG | 2313 |
| ATOM | 2313 | N | GLU | 284 | 13.842 | 20.030 | 36.228 | 1.00 | 0.36 | 1SG | 2314 |
| ATOM | 2314 | CA | GLU | 284 | 14.727 | 19.900 | 35.112 | 1.00 | 0.36 | 1SG | 2315 |
| ATOM | 2315 | CB | GLU | 284 | 15.290 | 21.232 | 34.582 | 1.00 | 0.36 | 1SG | 2316 |
| ATOM | 2316 | CG | GLU | 284 | 14.461 | 21.875 | 33.463 | 1.00 | 0.36 | 1SG | 2317 |
| ATOM | 2317 | CD | GLU | 284 | 13.033 | 22.093 | 33.921 | 1.00 | 0.36 | 1SG | 2318 |
| ATOM | 2318 | OE1 | GLU | 284 | 12.383 | 21.094 | 34.328 | 1.00 | 0.36 | 1SG | 2319 |
| ATOM | 2319 | OE2 | GLU | 284 | 12.570 | 23.263 | 33.874 | 1.00 | 0.36 | 1SG | 2320 |
| ATOM | 2320 | C | GLU | 284 | 15.880 | 19.030 | 35.488 | 1.00 | 0.36 | 1SG | 2321 |
| ATOM | 2321 | O | GLU | 284 | 16.251 | 18.138 | 34.727 | 1.00 | 0.36 | 1SG | 2322 |
| ATOM | 2322 | N | VAL | 285 | 16.487 | 19.255 | 36.670 | 1.00 | 0.31 | 1SG | 2323 |
| ATOM | 2323 | CA | VAL | 285 | 17.622 | 18.447 | 37.000 | 1.00 | 0.31 | 1SG | 2324 |
| ATOM | 2324 | CB | VAL | 285 | 18.465 | 19.004 | 38.125 | 1.00 | 0.31 | 1SG | 2325 |
| ATOM | 2325 | CG1 | VAL | 285 | 19.364 | 20.088 | 37.504 | 1.00 | 0.31 | 1SG | 2326 |
| ATOM | 2326 | CG2 | VAL | 285 | 17.569 | 19.658 | 39.194 | 1.00 | 0.31 | 1SG | 2327 |
| ATOM | 2327 | C | VAL | 285 | 17.253 | 16.993 | 37.148 | 1.00 | 0.31 | 1SG | 2328 |
| ATOM | 2328 | O | VAL | 285 | 17.895 | 16.173 | 36.492 | 1.00 | 0.31 | 1SG | 2329 |
| ATOM | 2329 | N | PRO | 286 | 16.275 | 16.573 | 37.913 | 1.00 | 0.23 | 1SG | 2330 |
| ATOM | 2330 | CA | PRO | 286 | 15.967 | 15.178 | 37.826 | 1.00 | 0.23 | 1SG | 2331 |
| ATOM | 2331 | CD | PRO | 286 | 16.224 | 16.978 | 39.312 | 1.00 | 0.23 | 1SG | 2332 |
| ATOM | 2332 | CB | PRO | 286 | 15.180 | 14.813 | 39.082 | 1.00 | 0.23 | 1SG | 2333 |
| ATOM | 2333 | CG | PRO | 286 | 15.765 | 15.762 | 40.131 | 1.00 | 0.23 | 1SG | 2334 |
| ATOM | 2334 | C | PRO | 286 | 15.301 | 14.784 | 36.556 | 1.00 | 0.23 | 1SG | 2335 |
| ATOM | 2335 | O | PRO | 286 | 15.158 | 13.592 | 36.327 | 1.00 | 0.23 | 1SG | 2336 |
| ATOM | 2336 | N | GLY | 287 | 14.806 | 15.710 | 35.730 | 1.00 | 0.23 | 1SG | 2337 |
| ATOM | 2337 | CA | GLY | 287 | 14.257 | 15.214 | 34.503 | 1.00 | 0.23 | 1SG | 2338 |
| ATOM | 2338 | C | GLY | 287 | 15.415 | 14.710 | 33.711 | 1.00 | 0.23 | 1SG | 2339 |
| ATOM | 2339 | O | GLY | 287 | 15.377 | 13.652 | 33.085 | 1.00 | 0.23 | 1SG | 2340 |
| ATOM | 2340 | N | SER | 288 | 16.494 | 15.504 | 33.758 | 1.00 | 0.47 | 1SG | 2341 |
| ATOM | 2341 | CA | SER | 288 | 17.710 | 15.296 | 33.041 | 1.00 | 0.47 | 1SG | 2342 |
| ATOM | 2342 | CB | SER | 288 | 18.679 | 16.465 | 33.262 | 1.00 | 0.47 | 1SG | 2343 |
| ATOM | 2343 | OG | SER | 288 | 19.951 | 16.146 | 32.728 | 1.00 | 0.47 | 1SG | 2344 |
| ATOM | 2344 | C | SER | 288 | 18.404 | 14.058 | 33.506 | 1.00 | 0.47 | 1SG | 2345 |
| ATOM | 2345 | O | SER | 288 | 18.964 | 13.331 | 32.687 | 1.00 | 0.47 | 1SG | 2346 |
| ATOM | 2346 | N | PHE | 289 | 18.408 | 13.765 | 34.824 | 1.00 | 0.65 | 1SG | 2347 |
| ATOM | 2347 | CA | PHE | 289 | 19.240 | 12.649 | 35.165 | 1.00 | 0.65 | 1SG | 2348 |
| ATOM | 2348 | CB | PHE | 289 | 19.699 | 12.536 | 36.648 | 1.00 | 0.65 | 1SG | 2349 |
| ATOM | 2349 | CG | PHE | 289 | 18.633 | 11.930 | 37.452 | 1.00 | 0.65 | 1SG | 2350 |
| ATOM | 2350 | CD1 | PHE | 289 | 17.603 | 12.684 | 37.954 | 1.00 | 0.65 | 1SG | 2351 |
| ATOM | 2351 | CD2 | PHE | 289 | 18.656 | 10.573 | 37.659 | 1.00 | 0.65 | 1SG | 2352 |
| ATOM | 2352 | CE1 | PHE | 289 | 16.603 | 12.079 | 38.671 | 1.00 | 0.65 | 1SG | 2353 |
| ATOM | 2353 | CE2 | PHE | 289 | 17.661 | 9.968 | 38.377 | 1.00 | 0.65 | 1SG | 2354 |
| ATOM | 2354 | CZ | PHE | 289 | 16.632 | 10.720 | 38.881 | 1.00 | 0.65 | 1SG | 2355 |
| ATOM | 2355 | C | PHE | 289 | 18.687 | 11.366 | 34.576 | 1.00 | 0.65 | 1SG | 2356 |
| ATOM | 2356 | O | PHE | 289 | 19.505 | 10.550 | 34.156 | 1.00 | 0.65 | 1SG | 2357 |
| ATOM | 2357 | N | PRO | 290 | 17.398 | 11.073 | 34.480 | 1.00 | 0.46 | 1SG | 2358 |
| ATOM | 2358 | CA | PRO | 290 | 17.036 | 9.887 | 33.766 | 1.00 | 0.46 | 1SG | 2359 |
| ATOM | 2359 | CD | PRO | 290 | 16.448 | 11.271 | 35.548 | 1.00 | 0.46 | 1SG | 2360 |
| ATOM | 2360 | CB | PRO | 290 | 15.679 | 9.406 | 34.282 | 1.00 | 0.46 | 1SG | 2361 |
| ATOM | 2361 | CG | PRO | 290 | 15.158 | 10.573 | 35.116 | 1.00 | 0.46 | 1SG | 2362 |
| ATOM | 2362 | C | PRO | 290 | 17.032 | 10.139 | 32.296 | 1.00 | 0.46 | 1SG | 2363 |
| ATOM | 2363 | O | PRO | 290 | 16.719 | 9.207 | 31.562 | 1.00 | 0.46 | 1SG | 2364 |
| ATOM | 2364 | N | SER | 291 | 17.385 | 11.365 | 31.855 | 1.00 | 0.24 | 1SG | 2365 |
| ATOM | 2365 | CA | SER | 291 | 17.392 | 11.766 | 30.471 | 1.00 | 0.24 | 1SG | 2366 |
| ATOM | 2366 | CB | SER | 291 | 18.290 | 10.920 | 29.540 | 1.00 | 0.24 | 1SG | 2367 |
| ATOM | 2367 | OG | SER | 291 | 17.700 | 9.666 | 29.234 | 1.00 | 0.24 | 1SG | 2368 |
| ATOM | 2368 | C | SER | 291 | 16.001 | 11.796 | 29.923 | 1.00 | 0.24 | 1SG | 2369 |
| ATOM | 2369 | O | SER | 291 | 15.732 | 11.300 | 28.830 | 1.00 | 0.24 | 1SG | 2370 |
| ATOM | 2370 | N | SER | 292 | 15.075 | 12.393 | 30.695 | 1.00 | 0.17 | 1SG | 2371 |
| ATOM | 2371 | CA | SER | 292 | 13.723 | 12.599 | 30.263 | 1.00 | 0.17 | 1SG | 2372 |
| ATOM | 2372 | CB | SER | 292 | 12.741 | 12.779 | 31.432 | 1.00 | 0.17 | 1SG | 2373 |
| ATOM | 2373 | OG | SER | 292 | 12.682 | 11.591 | 32.207 | 1.00 | 0.17 | 1SG | 2374 |
| ATOM | 2374 | C | SER | 292 | 13.742 | 13.880 | 29.493 | 1.00 | 0.17 | 1SG | 2375 |
| ATOM | 2375 | O | SER | 292 | 14.791 | 14.511 | 29.378 | 1.00 | 0.17 | 1SG | 2376 |
| ATOM | 2376 | N | ASN | 293 | 12.581 | 14.291 | 28.938 | 1.00 | 0.24 | 1SG | 2377 |
| ATOM | 2377 | CA | ASN | 293 | 12.516 | 15.500 | 28.165 | 1.00 | 0.24 | 1SG | 2378 |
| ATOM | 2378 | CB | ASN | 293 | 11.135 | 15.726 | 27.515 | 1.00 | 0.24 | 1SG | 2379 |
| ATOM | 2379 | CG | ASN | 293 | 10.066 | 15.739 | 28.598 | 1.00 | 0.24 | 1SG | 2380 |
| ATOM | 2380 | OD1 | ASN | 293 | 10.315 | 15.350 | 29.738 | 1.00 | 0.24 | 1SG | 2381 |
| ATOM | 2381 | ND2 | ASN | 293 | 8.836 | 16.186 | 28.232 | 1.00 | 0.24 | 1SG | 2382 |
| ATOM | 2382 | C | ASN | 293 | 12.882 | 16.664 | 29.036 | 1.00 | 0.24 | 1SG | 2383 |
| ATOM | 2383 | O | ASN | 293 | 12.049 | 17.248 | 29.727 | 1.00 | 0.24 | 1SG | 2384 |
| ATOM | 2384 | N | LYS | 294 | 14.180 | 17.023 | 29.020 | 1.00 | 0.29 | 1SG | 2385 |
| ATOM | 2385 | CA | LYS | 294 | 14.693 | 18.106 | 29.804 | 1.00 | 0.29 | 1SG | 2386 |
| ATOM | 2386 | CB | LYS | 294 | 16.217 | 18.244 | 29.638 | 1.00 | 0.29 | 1SG | 2387 |
| ATOM | 2387 | CG | LYS | 294 | 16.862 | 19.378 | 30.439 | 1.00 | 0.29 | 1SG | 2388 |
| ATOM | 2388 | CD | LYS | 294 | 17.019 | 19.107 | 31.935 | 1.00 | 0.29 | 1SG | 2389 |
| ATOM | 2389 | CE | LYS | 294 | 17.672 | 20.277 | 32.673 | 1.00 | 0.29 | 1SG | 2390 |
| ATOM | 2390 | NZ | LYS | 294 | 18.225 | 19.824 | 33.966 | 1.00 | 0.29 | 1SG | 2391 |
| ATOM | 2391 | C | LYS | 294 | 14.101 | 19.379 | 29.309 | 1.00 | 0.29 | 1SG | 2392 |
| ATOM | 2392 | O | LYS | 294 | 13.524 | 20.155 | 30.069 | 1.00 | 0.29 | 1SG | 2393 |
| ATOM | 2393 | N | TYR | 295 | 14.226 | 19.602 | 27.991 | 1.00 | 0.27 | 1SG | 2394 |
| ATOM | 2394 | CA | TYR | 295 | 13.797 | 20.815 | 27.368 | 1.00 | 0.27 | 1SG | 2395 |
| ATOM | 2395 | CB | TYR | 295 | 14.242 | 20.894 | 25.897 | 1.00 | 0.27 | 1SG | 2396 |
| ATOM | 2396 | CG | TYR | 295 | 13.847 | 22.224 | 25.357 | 1.00 | 0.27 | 1SG | 2397 |
| ATOM | 2397 | CD1 | TYR | 295 | 12.566 | 22.457 | 24.916 | 1.00 | 0.27 | 1SG | 2398 |
| ATOM | 2398 | CD2 | TYR | 295 | 14.772 | 23.241 | 25.287 | 1.00 | 0.27 | 1SG | 2399 |
| ATOM | 2399 | CE1 | TYR | 295 | 12.211 | 23.688 | 24.418 | 1.00 | 0.27 | 1SG | 2400 |
| ATOM | 2400 | CE2 | TYR | 295 | 14.424 | 24.474 | 24.790 | 1.00 | 0.27 | 1SG | 2401 |
| ATOM | 2401 | CZ | TYR | 295 | 13.141 | 24.698 | 24.354 | 1.00 | 0.27 | 1SG | 2402 |
| ATOM | 2402 | OH | TYR | 295 | 12.781 | 25.963 | 23.843 | 1.00 | 0.27 | 1SG | 2403 |
| ATOM | 2403 | C | TYR | 295 | 12.307 | 20.892 | 27.404 | 1.00 | 0.27 | 1SG | 2404 |
| ATOM | 2404 | O | TYR | 295 | 11.740 | 21.959 | 27.629 | 1.00 | 0.27 | 1SG | 2405 |
| ATOM | 2405 | N | ASP | 296 | 11.627 | 19.750 | 27.192 | 1.00 | 0.36 | 1SG | 2406 |
| ATOM | 2406 | CA | ASP | 296 | 10.200 | 19.809 | 27.100 | 1.00 | 0.36 | 1SG | 2407 |
| ATOM | 2407 | CB | ASP | 296 | 9.527 | 18.494 | 26.677 | 1.00 | 0.36 | 1SG | 2408 |
| ATOM | 2408 | CG | ASP | 296 | 9.555 | 18.456 | 25.161 | 1.00 | 0.36 | 1SG | 2409 |
| ATOM | 2409 | OD1 | ASP | 296 | 9.222 | 19.511 | 24.556 | 1.00 | 0.36 | 1SG | 2410 |
| ATOM | 2410 | OD2 | ASP | 296 | 9.906 | 17.391 | 24.588 | 1.00 | 0.36 | 1SG | 2411 |
| ATOM | 2411 | C | ASP | 296 | 9.590 | 20.255 | 28.379 | 1.00 | 0.36 | 1SG | 2412 |
| ATOM | 2412 | O | ASP | 296 | 9.813 | 19.679 | 29.442 | 1.00 | 0.36 | 1SG | 2413 |
| ATOM | 2413 | N | LEU | 297 | 8.800 | 21.339 | 28.276 | 1.00 | 0.55 | 1SG | 2414 |
| ATOM | 2414 | CA | LEU | 297 | 8.050 | 21.848 | 29.379 | 1.00 | 0.55 | 1SG | 2415 |
| ATOM | 2415 | CB | LEU | 297 | 8.656 | 23.088 | 30.065 | 1.00 | 0.55 | 1SG | 2416 |
| ATOM | 2416 | CG | LEU | 297 | 9.846 | 22.793 | 31.001 | 1.00 | 0.55 | 1SG | 2417 |
| ATOM | 2417 | CD2 | LEU | 297 | 11.016 | 22.130 | 30.267 | 1.00 | 0.55 | 1SG | 2418 |
| ATOM | 2418 | CD1 | LEU | 297 | 9.393 | 21.987 | 32.228 | 1.00 | 0.55 | 1SG | 2419 |
| ATOM | 2419 | C | LEU | 297 | 6.719 | 22.243 | 28.844 | 1.00 | 0.55 | 1SG | 2420 |
| ATOM | 2420 | O | LEU | 297 | 6.623 | 22.874 | 27.792 | 1.00 | 0.55 | 1SG | 2421 |
| ATOM | 2421 | N | SER | 298 | 5.648 | 21.850 | 29.556 | 1.00 | 0.81 | 1SG | 2422 |
| ATOM | 2422 | CA | SER | 298 | 4.329 | 22.222 | 29.150 | 1.00 | 0.81 | 1SG | 2423 |
| ATOM | 2423 | CB | SER | 298 | 3.492 | 21.054 | 28.601 | 1.00 | 0.81 | 1SG | 2424 |
| ATOM | 2424 | OG | SER | 298 | 3.271 | 20.089 | 29.618 | 1.00 | 0.81 | 1SG | 2425 |
| ATOM | 2425 | C | SER | 298 | 3.645 | 22.745 | 30.367 | 1.00 | 0.81 | 1SG | 2426 |
| ATOM | 2426 | O | SER | 298 | 3.921 | 22.316 | 31.485 | 1.00 | 0.81 | 1SG | 2427 |
| ATOM | 2427 | N | ASP | 299 | 2.720 | 23.697 | 30.170 | 1.00 | 1.10 | 1SG | 2428 |
| ATOM | 2428 | CA | ASP | 299 | 2.056 | 24.341 | 31.263 | 1.00 | 1.10 | 1SG | 2429 |
| ATOM | 2429 | CB | ASP | 299 | 0.897 | 25.252 | 30.824 | 1.00 | 1.10 | 1SG | 2430 |
| ATOM | 2430 | CG | ASP | 299 | 1.431 | 26.397 | 29.979 | 1.00 | 1.10 | 1SG | 2431 |
| ATOM | 2431 | OD1 | ASP | 299 | 2.664 | 26.426 | 29.721 | 1.00 | 1.10 | 1SG | 2432 |
| ATOM | 2432 | OD2 | ASP | 299 | 0.606 | 27.259 | 29.575 | 1.00 | 1.10 | 1SG | 2433 |
| ATOM | 2433 | C | ASP | 299 | 1.424 | 23.315 | 32.141 | 1.00 | 1.10 | 1SG | 2434 |
| ATOM | 2434 | O | ASP | 299 | 1.022 | 22.242 | 31.691 | 1.00 | 1.10 | 1SG | 2435 |
| ATOM | 2435 | N | MET | 300 | 1.345 | 23.640 | 33.446 | 1.00 | 1.22 | 1SG | 2436 |
| ATOM | 2436 | CA | MET | 300 | 0.649 | 22.821 | 34.392 | 1.00 | 1.22 | 1SG | 2437 |
| ATOM | 2437 | CB | MET | 300 | 1.543 | 21.979 | 35.320 | 1.00 | 1.22 | 1SG | 2438 |
| ATOM | 2438 | CG | MET | 300 | 0.723 | 21.032 | 36.203 | 1.00 | 1.22 | 1SG | 2439 |
| ATOM | 2439 | SD | MET | 300 | 1.697 | 19.856 | 37.190 | 1.00 | 1.22 | 1SG | 2440 |
| ATOM | 2440 | CE | MET | 300 | 2.345 | 21.105 | 38.336 | 1.00 | 1.22 | 1SG | 2441 |
| ATOM | 2441 | C | MET | 300 | −0.122 | 23.779 | 35.240 | 1.00 | 1.22 | 1SG | 2442 |
| ATOM | 2442 | O | MET | 300 | 0.198 | 24.966 | 35.275 | 1.00 | 1.22 | 1SG | 2443 |
| ATOM | 2443 | N | ASN | 301 | −1.179 | 23.309 | 35.928 | 1.00 | 1.05 | 1SG | 2444 |
| ATOM | 2444 | CA | ASN | 301 | −1.921 | 24.249 | 36.710 | 1.00 | 1.05 | 1SG | 2445 |
| ATOM | 2445 | CB | ASN | 301 | −3.090 | 24.874 | 35.934 | 1.00 | 1.05 | 1SG | 2446 |
| ATOM | 2446 | CG | ASN | 301 | −3.576 | 26.095 | 36.709 | 1.00 | 1.05 | 1SG | 2447 |
| ATOM | 2447 | OD1 | ASN | 301 | −2.896 | 27.120 | 36.749 | 1.00 | 1.05 | 1SG | 2448 |
| ATOM | 2448 | ND2 | ASN | 301 | −4.778 | 25.989 | 37.339 | 1.00 | 1.05 | 1SG | 2449 |
| ATOM | 2449 | C | ASN | 301 | −2.470 | 23.540 | 37.904 | 1.00 | 1.05 | 1SG | 2450 |
| ATOM | 2450 | O | ASN | 301 | −2.359 | 22.321 | 38.028 | 1.00 | 1.05 | 1SG | 2451 |
| ATOM | 2451 | N | ALA | 302 | −3.052 | 24.312 | 38.841 | 1.00 | 0.80 | 1SG | 2452 |
| ATOM | 2452 | CA | ALA | 302 | −3.606 | 23.749 | 40.035 | 1.00 | 0.80 | 1SG | 2453 |
| ATOM | 2453 | CB | ALA | 302 | −3.077 | 24.416 | 41.316 | 1.00 | 0.80 | 1SG | 2454 |
| ATOM | 2454 | C | ALA | 302 | −5.076 | 24.002 | 39.991 | 1.00 | 0.80 | 1SG | 2455 |
| ATOM | 2455 | O | ALA | 302 | −5.520 | 25.131 | 39.782 | 1.00 | 0.80 | 1SG | 2456 |

TABLE 5
Core 2L bovine

| | | | | | | | | | | | |
|---|---|---|---|---|---|---|---|---|---|---|---|
| ATOM | 437 | N | GLU | 50 | −19.105 | −2.890 | 13.088 | 1.00 | 0.68 | 1SG | 438 |
| ATOM | 438 | CA | GLU | 50 | −17.991 | −2.301 | 13.769 | 1.00 | 0.68 | 1SG | 439 |
| ATOM | 439 | CB | GLU | 50 | −17.473 | −3.211 | 14.892 | 1.00 | 0.68 | 1SG | 440 |
| ATOM | 440 | CG | GLU | 50 | −18.588 | −3.865 | 15.709 | 1.00 | 0.68 | 1SG | 441 |
| ATOM | 441 | CD | GLU | 50 | −18.879 | −5.219 | 15.055 | 1.00 | 0.68 | 1SG | 442 |
| ATOM | 442 | OE1 | GLU | 50 | −17.952 | −6.089 | 15.039 | 1.00 | 0.68 | 1SG | 443 |
| ATOM | 443 | OE2 | GLU | 50 | −20.023 | −5.404 | 14.564 | 1.00 | 0.68 | 1SG | 444 |
| ATOM | 444 | C | GLU | 50 | −16.845 | −2.169 | 12.816 | 1.00 | 0.68 | 1SG | 445 |
| ATOM | 445 | O | GLU | 50 | −16.035 | −3.087 | 12.694 | 1.00 | 0.68 | 1SG | 446 |
| ATOM | 446 | N | GLU | 51 | −16.719 | −1.027 | 12.113 | 1.00 | 0.76 | 1SG | 447 |
| ATOM | 447 | CA | GLU | 51 | −15.573 | −0.915 | 11.260 | 1.00 | 0.76 | 1SG | 448 |
| ATOM | 448 | CB | GLU | 51 | −15.891 | −1.135 | 9.763 | 1.00 | 0.76 | 1SG | 449 |
| ATOM | 449 | CG | GLU | 51 | −16.842 | −0.120 | 9.120 | 1.00 | 0.76 | 1SG | 450 |
| ATOM | 450 | CD | GLU | 51 | −16.030 | 0.792 | 8.209 | 1.00 | 0.76 | 1SG | 451 |
| ATOM | 451 | OE1 | GLU | 51 | −15.488 | 0.276 | 7.196 | 1.00 | 0.76 | 1SG | 452 |
| ATOM | 452 | OE2 | GLU | 51 | −15.953 | 2.015 | 8.509 | 1.00 | 0.76 | 1SG | 453 |
| ATOM | 453 | C | GLU | 51 | −14.983 | 0.443 | 11.450 | 1.00 | 0.76 | 1SG | 454 |
| ATOM | 454 | O | GLU | 51 | −15.648 | 1.452 | 11.219 | 1.00 | 0.76 | 1SG | 455 |
| ATOM | 455 | N | ASN | 52 | −13.711 | 0.506 | 11.899 | 1.00 | 0.73 | 1SG | 456 |
| ATOM | 456 | CA | ASN | 52 | −13.104 | 1.793 | 12.067 | 1.00 | 0.73 | 1SG | 457 |
| ATOM | 457 | CB | ASN | 52 | −12.566 | 2.076 | 13.484 | 1.00 | 0.73 | 1SG | 458 |
| ATOM | 458 | CG | ASN | 52 | −11.410 | 1.147 | 13.807 | 1.00 | 0.73 | 1SG | 459 |
| ATOM | 459 | OD1 | ASN | 52 | −10.256 | 1.564 | 13.745 | 1.00 | 0.73 | 1SG | 460 |
| ATOM | 460 | ND2 | ASN | 52 | −11.709 | −0.126 | 14.178 | 1.00 | 0.73 | 1SG | 461 |
| ATOM | 461 | C | ASN | 52 | −11.965 | 1.883 | 11.105 | 1.00 | 0.73 | 1SG | 462 |
| ATOM | 462 | O | ASN | 52 | −11.055 | 1.055 | 11.089 | 1.00 | 0.73 | 1SG | 463 |
| ATOM | 463 | N | PRO | 53 | −12.037 | 2.879 | 10.273 | 1.00 | 0.68 | 1SG | 464 |
| ATOM | 464 | CA | PRO | 53 | −11.004 | 3.051 | 9.290 | 1.00 | 0.68 | 1SG | 465 |
| ATOM | 465 | CD | PRO | 53 | −13.337 | 3.286 | 9.764 | 1.00 | 0.68 | 1SG | 466 |
| ATOM | 466 | CB | PRO | 53 | −11.619 | 3.874 | 8.162 | 1.00 | 0.68 | 1SG | 467 |
| ATOM | 467 | CG | PRO | 53 | −13.123 | 3.585 | 8.273 | 1.00 | 0.68 | 1SG | 468 |
| ATOM | 468 | C | PRO | 53 | −9.788 | 3.700 | 9.857 | 1.00 | 0.68 | 1SG | 469 |
| ATOM | 469 | O | PRO | 53 | −9.903 | 4.478 | 10.804 | 1.00 | 0.68 | 1SG | 470 |
| ATOM | 470 | N | SER | 54 | −8.612 | 3.386 | 9.288 | 1.00 | 0.54 | 1SG | 471 |
| ATOM | 471 | CA | SER | 54 | −7.414 | 4.045 | 9.704 | 1.00 | 0.54 | 1SG | 472 |
| ATOM | 472 | CB | SER | 54 | −6.205 | 3.103 | 9.847 | 1.00 | 0.54 | 1SG | 473 |
| ATOM | 473 | OG | SER | 54 | −5.895 | 2.516 | 8.591 | 1.00 | 0.54 | 1SG | 474 |
| ATOM | 474 | C | SER | 54 | −7.110 | 5.001 | 8.600 | 1.00 | 0.54 | 1SG | 475 |
| ATOM | 475 | O | SER | 54 | −6.941 | 4.604 | 7.447 | 1.00 | 0.54 | 1SG | 476 |
| ATOM | 476 | N | SER | 55 | −7.055 | 6.305 | 8.916 | 1.00 | 0.43 | 1SG | 477 |
| ATOM | 477 | CA | SER | 55 | −6.796 | 7.219 | 7.848 | 1.00 | 0.43 | 1SG | 478 |
| ATOM | 478 | CB | SER | 55 | −7.698 | 8.464 | 7.866 | 1.00 | 0.43 | 1SG | 479 |
| ATOM | 479 | OG | SER | 55 | −7.375 | 9.316 | 6.779 | 1.00 | 0.43 | 1SG | 480 |
| ATOM | 480 | C | SER | 55 | −5.373 | 7.658 | 7.965 | 1.00 | 0.43 | 1SG | 481 |
| ATOM | 481 | O | SER | 55 | −4.952 | 8.191 | 8.991 | 1.00 | 0.43 | 1SG | 482 |
| ATOM | 482 | N | ASN | 56 | −4.596 | 7.424 | 6.889 | 1.00 | 0.48 | 1SG | 483 |
| ATOM | 483 | CA | ASN | 56 | −3.204 | 7.770 | 6.855 | 1.00 | 0.48 | 1SG | 484 |
| ATOM | 484 | CB | ASN | 56 | −2.344 | 6.744 | 6.090 | 1.00 | 0.48 | 1SG | 485 |
| ATOM | 485 | CG | ASN | 56 | −0.877 | 7.149 | 6.185 | 1.00 | 0.48 | 1SG | 486 |
| ATOM | 486 | OD1 | ASN | 56 | −0.331 | 7.310 | 7.276 | 1.00 | 0.48 | 1SG | 487 |
| ATOM | 487 | ND2 | ASN | 56 | −0.214 | 7.305 | 5.007 | 1.00 | 0.48 | 1SG | 488 |
| ATOM | 488 | C | ASN | 56 | −3.082 | 9.072 | 6.132 | 1.00 | 0.48 | 1SG | 489 |
| ATOM | 489 | O | ASN | 56 | −3.690 | 9.279 | 5.082 | 1.00 | 0.48 | 1SG | 490 |
| ATOM | 490 | N | ILE | 57 | −2.281 | 9.995 | 6.700 | 1.00 | 0.63 | 1SG | 491 |
| ATOM | 491 | CA | ILE | 57 | −2.096 | 11.287 | 6.109 | 1.00 | 0.63 | 1SG | 492 |
| ATOM | 492 | CB | ILE | 57 | −1.994 | 12.370 | 7.140 | 1.00 | 0.63 | 1SG | 493 |
| ATOM | 493 | CG2 | ILE | 57 | −2.007 | 13.724 | 6.411 | 1.00 | 0.63 | 1SG | 494 |
| ATOM | 494 | CG1 | ILE | 57 | −3.089 | 12.211 | 8.207 | 1.00 | 0.63 | 1SG | 495 |
| ATOM | 495 | CD1 | ILE | 57 | −4.496 | 12.057 | 7.652 | 1.00 | 0.63 | 1SG | 496 |
| ATOM | 496 | C | ILE | 57 | −0.739 | 11.220 | 5.489 | 1.00 | 0.63 | 1SG | 497 |
| ATOM | 497 | O | ILE | 57 | 0.222 | 10.866 | 6.170 | 1.00 | 0.63 | 1SG | 498 |
| ATOM | 498 | N | ASN | 58 | −0.600 | 11.534 | 4.185 | 1.00 | 0.90 | 1SG | 499 |
| ATOM | 499 | CA | ASN | 58 | 0.733 | 11.422 | 3.670 | 1.00 | 0.90 | 1SG | 500 |
| ATOM | 500 | CB | ASN | 58 | 1.090 | 10.002 | 3.196 | 1.00 | 0.90 | 1SG | 501 |
| ATOM | 501 | CG | ASN | 58 | −0.204 | 9.663 | 2.004 | 1.00 | 0.90 | 1SG | 502 |
| ATOM | 502 | OD1 | ASN | 58 | 1.020 | 9.770 | 2.061 | 1.00 | 0.90 | 1SG | 503 |
| ATOM | 503 | ND2 | ASN | 58 | 0.845 | 9.245 | 0.880 | 1.00 | 0.90 | 1SG | 504 |
| ATOM | 504 | C | ASN | 58 | 0.934 | 12.317 | 2.493 | 1.00 | 0.90 | 1SG | 505 |
| ATOM | 505 | O | ASN | 58 | −0.001 | 12.941 | 1.990 | 1.00 | 0.90 | 1SG | 506 |
| ATOM | 506 | N | CYS | 59 | 2.220 | 12.409 | 2.085 | 1.00 | 1.07 | 1SG | 507 |
| ATOM | 507 | CA | CYS | 59 | 2.725 | 13.071 | 0.915 | 1.00 | 1.07 | 1SG | 508 |
| ATOM | 508 | CB | CYS | 59 | 1.671 | 13.507 | −0.102 | 1.00 | 1.07 | 1SG | 509 |
| ATOM | 509 | SG | CYS | 59 | 2.453 | 13.974 | −1.671 | 1.00 | 1.07 | 1SG | 510 |
| ATOM | 510 | C | CYS | 59 | 3.501 | 14.279 | 1.305 | 1.00 | 1.07 | 1SG | 511 |
| ATOM | 511 | O | CYS | 59 | 4.142 | 14.905 | 0.462 | 1.00 | 1.07 | 1SG | 512 |
| ATOM | 512 | N | THR | 60 | 3.474 | 14.643 | 2.596 | 1.00 | 0.84 | 1SG | 513 |
| ATOM | 513 | CA | THR | 60 | 4.219 | 15.793 | 3.001 | 1.00 | 0.84 | 1SG | 514 |
| ATOM | 514 | CB | THR | 60 | 4.003 | 16.145 | 4.444 | 1.00 | 0.84 | 1SG | 515 |
| ATOM | 515 | OG1 | THR | 60 | 2.631 | 16.426 | 4.681 | 1.00 | 0.84 | 1SG | 516 |
| ATOM | 516 | CG2 | THR | 60 | 4.862 | 17.375 | 4.786 | 1.00 | 0.84 | 1SG | 517 |
| ATOM | 517 | C | THR | 60 | 5.664 | 15.473 | 2.829 | 1.00 | 0.84 | 1SG | 518 |
| ATOM | 518 | O | THR | 60 | 6.424 | 16.254 | 2.259 | 1.00 | 0.84 | 1SG | 519 |
| ATOM | 519 | N | LYS | 61 | 6.076 | 14.281 | 3.295 | 1.00 | 0.47 | 1SG | 520 |
| ATOM | 520 | CA | LYS | 61 | 7.461 | 13.944 | 3.200 | 1.00 | 0.47 | 1SG | 521 |
| ATOM | 521 | CB | LYS | 61 | 8.027 | 13.330 | 4.492 | 1.00 | 0.47 | 1SG | 522 |
| ATOM | 522 | CG | LYS | 61 | 8.030 | 14.308 | 5.671 | 1.00 | 0.47 | 1SG | 523 |
| ATOM | 523 | CD | LYS | 61 | 8.235 | 13.637 | 7.031 | 1.00 | 0.47 | 1SG | 524 |
| ATOM | 524 | CE | LYS | 61 | 9.677 | 13.197 | 7.292 | 1.00 | 0.47 | 1SG | 525 |
| ATOM | 525 | NZ | LYS | 61 | 9.778 | 12.563 | 8.626 | 1.00 | 0.47 | 1SG | 526 |
| ATOM | 526 | C | LYS | 61 | 7.610 | 12.952 | 2.103 | 1.00 | 0.47 | 1SG | 527 |
| ATOM | 527 | O | LYS | 61 | 6.865 | 11.976 | 2.019 | 1.00 | 0.47 | 1SG | 528 |
| ATOM | 528 | N | ILE | 62 | 8.585 | 13.205 | 1.212 | 1.00 | 0.58 | 1SG | 529 |
| ATOM | 529 | CA | ILE | 62 | 8.826 | 12.325 | 0.114 | 1.00 | 0.58 | 1SG | 530 |
| ATOM | 530 | CB | ILE | 62 | 8.925 | 13.034 | −1.204 | 1.00 | 0.58 | 1SG | 531 |
| ATOM | 531 | CG2 | ILE | 62 | 9.338 | 12.008 | −2.272 | 1.00 | 0.58 | 1SG | 532 |
| ATOM | 532 | CG1 | ILE | 62 | 7.610 | 13.764 | −1.520 | 1.00 | 0.58 | 1SG | 533 |
| ATOM | 533 | CD1 | ILE | 62 | 7.723 | 14.740 | −2.689 | 1.00 | 0.58 | 1SG | 534 |
| ATOM | 534 | C | ILE | 62 | 10.146 | 11.682 | 0.366 | 1.00 | 0.58 | 1SG | 535 |
| ATOM | 535 | O | ILE | 62 | 11.063 | 12.305 | 0.897 | 1.00 | 0.58 | 1SG | 536 |
| ATOM | 536 | N | LEU | 63 | 10.265 | 10.393 | 0.005 | 1.00 | 1.10 | 1SG | 537 |
| ATOM | 537 | CA | LEU | 63 | 11.501 | 9.702 | 0.200 | 1.00 | 1.10 | 1SG | 538 |
| ATOM | 538 | CB | LEU | 63 | 11.357 | 8.439 | 1.065 | 1.00 | 1.10 | 1SG | 539 |
| ATOM | 539 | CG | LEU | 63 | 12.675 | 7.673 | 1.296 | 1.00 | 1.10 | 1SG | 540 |
| ATOM | 540 | CD2 | LEU | 63 | 12.420 | 6.316 | 1.975 | 1.00 | 1.10 | 1SG | 541 |
| ATOM | 541 | CD1 | LEU | 63 | 13.693 | 8.532 | 2.055 | 1.00 | 1.10 | 1SG | 542 |
| ATOM | 542 | C | LEU | 63 | 11.967 | 9.291 | −1.155 | 1.00 | 1.10 | 1SG | 543 |
| ATOM | 543 | O | LEU | 63 | 11.242 | 9.438 | −2.139 | 1.00 | 1.10 | 1SG | 544 |
| ATOM | 544 | N | GLN | 64 | 13.210 | 8.783 | −1.247 | 1.00 | 1.13 | 1SG | 545 |
| ATOM | 545 | CA | GLN | 64 | 13.699 | 8.375 | −2.525 | 1.00 | 1.13 | 1SG | 546 |
| ATOM | 546 | CB | GLN | 64 | 15.114 | 7.778 | −2.491 | 1.00 | 1.13 | 1SG | 547 |
| ATOM | 547 | CG | GLN | 64 | 15.618 | 7.353 | −3.870 | 1.00 | 1.13 | 1SG | 548 |
| ATOM | 548 | CD | GLN | 64 | 15.765 | 8.613 | −4.710 | 1.00 | 1.13 | 1SG | 549 |
| ATOM | 549 | OE1 | GLN | 64 | 15.499 | 9.719 | −4.241 | 1.00 | 1.13 | 1SG | 550 |
| ATOM | 550 | NE2 | GLN | 64 | 16.199 | 8.446 | −5.988 | 1.00 | 1.13 | 1SG | 551 |
| ATOM | 551 | C | GLN | 64 | 12.766 | 7.335 | −3.035 | 1.00 | 1.13 | 1SG | 552 |
| ATOM | 552 | O | GLN | 64 | 11.997 | 6.751 | −2.275 | 1.00 | 1.13 | 1SG | 553 |
| ATOM | 553 | N | GLY | 65 | 12.801 | 7.089 | −4.355 | 1.00 | 0.61 | 1SG | 554 |
| ATOM | 554 | CA | GLY | 65 | 11.921 | 6.116 | −4.923 | 1.00 | 0.61 | 1SG | 555 |
| ATOM | 555 | C | GLY | 65 | 10.553 | 6.708 | −4.873 | 1.00 | 0.61 | 1SG | 556 |
| ATOM | 556 | O | GLY | 65 | 10.343 | 7.764 | −4.279 | 1.00 | 0.61 | 1SG | 557 |
| ATOM | 557 | N | ASP | 66 | 9.578 | 6.031 | −5.505 | 1.00 | 0.37 | 1SG | 558 |
| ATOM | 558 | CA | ASP | 66 | 8.240 | 6.538 | −5.483 | 1.00 | 0.37 | 1SG | 559 |
| ATOM | 559 | CB | ASP | 66 | 7.248 | 5.649 | −6.250 | 1.00 | 0.37 | 1SG | 560 |
| ATOM | 560 | CG | ASP | 66 | 7.554 | 5.765 | −7.735 | 1.00 | 0.37 | 1SG | 561 |
| ATOM | 561 | OD1 | ASP | 66 | 8.353 | 6.665 | −8.107 | 1.00 | 0.37 | 1SG | 562 |
| ATOM | 562 | OD2 | ASP | 66 | 6.994 | 4.952 | −8.518 | 1.00 | 0.37 | 1SG | 563 |
| ATOM | 563 | C | ASP | 66 | 7.821 | 6.546 | −4.055 | 1.00 | 0.37 | 1SG | 564 |
| ATOM | 564 | O | ASP | 66 | 7.268 | 7.527 | −3.559 | 1.00 | 0.37 | 1SG | 565 |
| ATOM | 565 | N | VAL | 67 | 8.105 | 5.439 | −3.347 | 1.00 | 0.47 | 1SG | 566 |
| ATOM | 566 | CA | VAL | 67 | 7.741 | 5.339 | −1.968 | 1.00 | 0.47 | 1SG | 567 |
| ATOM | 567 | CB | VAL | 67 | 8.175 | 4.047 | −1.347 | 1.00 | 0.47 | 1SG | 568 |
| ATOM | 568 | CG1 | VAL | 67 | 7.416 | 2.895 | −2.028 | 1.00 | 0.47 | 1SG | 569 |
| ATOM | 569 | CG2 | VAL | 67 | 9.704 | 3.948 | −1.489 | 1.00 | 0.47 | 1SG | 570 |
| ATOM | 570 | C | VAL | 67 | 8.424 | 6.449 | −1.243 | 1.00 | 0.47 | 1SG | 571 |
| ATOM | 571 | O | VAL | 67 | 9.426 | 6.987 | −1.711 | 1.00 | 0.47 | 1SG | 572 |
| ATOM | 572 | N | ASP | 68 | 7.875 | 6.834 | −0.075 | 1.00 | 0.59 | 1SG | 573 |
| ATOM | 573 | CA | ASP | 68 | 8.460 | 7.893 | 0.688 | 1.00 | 0.59 | 1SG | 574 |
| ATOM | 574 | CB | ASP | 68 | 7.503 | 9.080 | 0.893 | 1.00 | 0.59 | 1SG | 575 |
| ATOM | 575 | CG | ASP | 68 | 7.241 | 9.711 | −0.469 | 1.00 | 0.59 | 1SG | 576 |
| ATOM | 576 | OD1 | ASP | 68 | 7.932 | 9.315 | −1.446 | 1.00 | 0.59 | 1SG | 577 |
| ATOM | 577 | OD2 | ASP | 68 | 6.350 | 10.597 | −0.552 | 1.00 | 0.59 | 1SG | 578 |
| ATOM | 578 | C | ASP | 68 | 8.767 | 7.328 | 2.035 | 1.00 | 0.59 | 1SG | 579 |
| ATOM | 579 | O | ASP | 68 | 8.918 | 6.119 | 2.200 | 1.00 | 0.59 | 1SG | 580 |
| ATOM | 580 | N | GLU | 69 | 8.874 | 8.215 | 3.039 | 1.00 | 0.80 | 1SG | 581 |
| ATOM | 581 | CA | GLU | 69 | 9.137 | 7.809 | 4.387 | 1.00 | 0.80 | 1SG | 582 |
| ATOM | 582 | CB | GLU | 69 | 9.258 | 8.982 | 5.371 | 1.00 | 0.80 | 1SG | 583 |
| ATOM | 583 | CG | GLU | 69 | 7.930 | 9.700 | 5.616 | 1.00 | 0.80 | 1SG | 584 |
| ATOM | 584 | CD | GLU | 69 | 7.549 | 10.427 | 4.339 | 1.00 | 0.80 | 1SG | 585 |
| ATOM | 585 | OE1 | GUU | 69 | 8.478 | 10.755 | 3.553 | 1.00 | 0.80 | 1SG | 586 |
| ATOM | 586 | OE2 | GLU | 69 | 6.330 | 10.672 | 4.134 | 1.00 | 0.80 | 1SG | 587 |
| ATOM | 587 | C | GLU | 69 | 7.944 | 7.025 | 4.810 | 1.00 | 0.80 | 1SG | 588 |
| ATOM | 588 | O | GLU | 69 | 8.025 | 6.145 | 5.665 | 1.00 | 0.80 | 1SG | 589 |
| ATOM | 589 | N | ILE | 70 | 6.793 | 7.339 | 4.191 | 1.00 | 0.79 | 1SG | 590 |
| ATOM | 590 | CA | ILE | 70 | 5.549 | 6.736 | 4.549 | 1.00 | 0.79 | 1SG | 591 |
| ATOM | 591 | CB | ILE | 70 | 4.429 | 7.145 | 3.617 | 1.00 | 0.79 | 1SG | 592 |
| ATOM | 592 | CG2 | ILE | 70 | 4.405 | 8.678 | 3.607 | 1.00 | 0.79 | 1SG | 593 |
| ATOM | 593 | CG1 | ILE | 70 | 4.579 | 6.568 | 2.194 | 1.00 | 0.79 | 1SG | 594 |
| ATOM | 594 | CD1 | ILE | 70 | 4.034 | 5.146 | 2.025 | 1.00 | 0.79 | 1SG | 595 |
| ATOM | 595 | C | ILE | 70 | 5.731 | 5.260 | 4.431 | 1.00 | 0.79 | 1SG | 596 |
| ATOM | 596 | O | ILE | 70 | 5.234 | 4.497 | 5.256 | 1.00 | 0.79 | 1SG | 597 |
| ATOM | 597 | N | GLN | 71 | 6.470 | 4.815 | 3.403 | 1.00 | 0.60 | 1SG | 598 |
| ATOM | 598 | CA | GLN | 71 | 6.590 | 3.403 | 3.191 | 1.00 | 0.60 | 1SG | 599 |
| ATOM | 599 | CB | GLN | 71 | 7.419 | 3.045 | 1.950 | 1.00 | 0.60 | 1SG | 600 |
| ATOM | 600 | CG | GLN | 71 | 7.542 | 1.535 | 1.742 | 1.00 | 0.60 | 1SG | 601 |
| ATOM | 601 | CD | GLN | 71 | 8.471 | 1.293 | 0.564 | 1.00 | 0.60 | 1SG | 602 |
| ATOM | 602 | OE1 | GLN | 71 | 9.479 | 1.980 | 0.406 | 1.00 | 0.60 | 1SG | 603 |
| ATOM | 603 | NE2 | GLN | 71 | 8.130 | 0.286 | −0.283 | 1.00 | 0.60 | 1SG | 604 |
| ATOM | 604 | C | GLN | 71 | 7.264 | 2.740 | 4.345 | 1.00 | 0.60 | 1SG | 605 |
| ATOM | 605 | O | GLN | 71 | 6.766 | 1.742 | 4.863 | 1.00 | 0.60 | 1SG | 606 |
| ATOM | 606 | N | LYS | 72 | 8.411 | 3.276 | 4.800 | 1.00 | 0.59 | 1SG | 607 |
| ATOM | 607 | CA | LYS | 72 | 9.103 | 2.564 | 5.838 | 1.00 | 0.59 | 1SG | 608 |
| ATOM | 608 | CB | LYS | 72 | 10.593 | 2.910 | 5.943 | 1.00 | 0.59 | 1SG | 609 |
| ATOM | 609 | CG | LYS | 72 | 11.291 | 2.129 | 7.057 | 1.00 | 0.59 | 1SG | 610 |
| ATOM | 610 | CD | LYS | 72 | 12.819 | 2.184 | 7.008 | 1.00 | 0.59 | 1SG | 611 |
| ATOM | 611 | CE | LYS | 72 | 13.487 | 1.412 | 8.152 | 1.00 | 0.59 | 1SG | 612 |
| ATOM | 612 | NZ | LYS | 72 | 13.119 | 2.006 | 9.457 | 1.00 | 0.59 | 1SG | 613 |
| ATOM | 613 | C | LYS | 72 | 8.487 | 2.886 | 7.140 | 1.00 | 0.59 | 1SG | 614 |
| ATOM | 614 | O | LYS | 72 | 9.109 | 3.524 | 7.986 | 1.00 | 0.59 | 1SG | 615 |
| ATOM | 615 | N | VAL | 73 | 7.250 | 2.417 | 7.349 | 1.00 | 0.65 | 1SG | 616 |
| ATOM | 616 | CA | VAL | 73 | 6.656 | 2.736 | 8.596 | 1.00 | 0.65 | 1SG | 617 |
| ATOM | 617 | CB | VAL | 73 | 6.628 | 4.229 | 8.869 | 1.00 | 0.65 | 1SG | 618 |
| ATOM | 618 | CG1 | VAL | 73 | 5.630 | 4.926 | 7.932 | 1.00 | 0.65 | 1SG | 619 |
| ATOM | 619 | CG2 | VAL | 73 | 6.399 | 4.481 | 10.367 | 1.00 | 0.65 | 1SG | 620 |
| ATOM | 620 | C | VAL | 73 | 5.289 | 2.195 | 8.569 | 1.00 | 0.65 | 1SG | 621 |
| ATOM | 621 | O | VAL | 73 | 5.015 | 1.241 | 7.837 | 1.00 | 0.65 | 1SG | 622 |
| ATOM | 622 | N | LYS | 74 | 4.377 | 2.822 | 9.324 | 1.00 | 0.66 | 1SG | 623 |
| ATOM | 623 | CA | LYS | 74 | 3.034 | 2.380 | 9.479 | 1.00 | 0.66 | 1SG | 624 |
| ATOM | 624 | CB | LYS | 74 | 2.172 | 3.400 | 10.242 | 1.00 | 0.66 | 1SG | 625 |
| ATOM | 625 | CG | LYS | 74 | 2.627 | 3.561 | 11.695 | 1.00 | 0.66 | 1SG | 626 |
| ATOM | 626 | CD | LYS | 74 | 1.945 | 4.701 | 12.452 | 1.00 | 0.66 | 1SG | 627 |
| ATOM | 627 | CE | LYS | 74 | 0.699 | 4.257 | 13.223 | 1.00 | 0.66 | 1SG | 628 |
| ATOM | 628 | NZ | LYS | 74 | 0.131 | 5.409 | 13.963 | 1.00 | 0.66 | 1SG | 629 |
| ATOM | 629 | C | LYS | 74 | 2.442 | 2.129 | 8.133 | 1.00 | 0.66 | 1SG | 630 |
| ATOM | 630 | O | LYS | 74 | 1.459 | 1.406 | 8.028 | 1.00 | 0.66 | 1SG | 631 |
| ATOM | 631 | N | LEU | 75 | 2.963 | 2.741 | 7.055 | 1.00 | 0.67 | 1SG | 632 |
| ATOM | 632 | CA | LEU | 75 | 2.360 | 2.371 | 5.812 | 1.00 | 0.67 | 1SG | 633 |
| ATOM | 633 | CB | LEU | 75 | 2.942 | 3.146 | 4.610 | 1.00 | 0.67 | 1SG | 634 |
| ATOM | 634 | CG | LEU | 75 | 2.259 | 2.852 | 3.258 | 1.00 | 0.67 | 1SG | 635 |
| ATOM | 635 | CD2 | LEU | 75 | 0.753 | 3.142 | 3.353 | 1.00 | 0.67 | 1SG | 636 |
| ATOM | 636 | CD1 | LEU | 75 | 2.563 | 1.439 | 2.727 | 1.00 | 0.67 | 1SG | 637 |
| ATOM | 637 | C | LEU | 75 | 2.620 | 0.909 | 5.616 | 1.00 | 0.67 | 1SG | 638 |
| ATOM | 638 | O | LEU | 75 | 1.688 | 0.114 | 5.552 | 1.00 | 0.67 | 1SG | 639 |
| ATOM | 639 | N | GLU | 76 | 3.903 | 0.506 | 5.592 | 1.00 | 0.56 | 1SG | 640 |
| ATOM | 640 | CA | GLU | 76 | 4.247 | −0.872 | 5.392 | 1.00 | 0.56 | 1SG | 641 |
| ATOM | 641 | CB | GLU | 76 | 5.764 | −1.055 | 5.204 | 1.00 | 0.56 | 1SG | 642 |
| ATOM | 642 | CG | GLU | 76 | 6.193 | −2.449 | 4.745 | 1.00 | 0.56 | 1SG | 643 |
| ATOM | 643 | CD | GLU | 76 | 7.700 | −2.409 | 4.520 | 1.00 | 0.56 | 1SG | 644 |
| ATOM | 644 | OE1 | GLU | 76 | 8.297 | −1.316 | 4.716 | 1.00 | 0.56 | 1SG | 645 |
| ATOM | 645 | OE2 | GLU | 76 | 8.275 | −3.466 | 4.146 | 1.00 | 0.56 | 1SG | 646 |
| ATOM | 646 | C | GLU | 76 | 3.817 | −1.684 | 6.577 | 1.00 | 0.56 | 1SG | 647 |
| ATOM | 647 | O | GLU | 76 | 3.172 | −2.721 | 6.439 | 1.00 | 0.56 | 1SG | 648 |
| ATOM | 648 | N | SER | 77 | 4.155 | −1.204 | 7.788 | 1.00 | 0.43 | 1SG | 649 |
| ATOM | 649 | CA | SER | 77 | 3.898 | −1.909 | 9.006 | 1.00 | 0.43 | 1SG | 650 |
| ATOM | 650 | CB | SER | 77 | 4.483 | −1.189 | 10.233 | 1.00 | 0.43 | 1SG | 651 |
| ATOM | 651 | OG | SER | 77 | 5.898 | −1.133 | 10.129 | 1.00 | 0.43 | 1SG | 652 |
| ATOM | 652 | C | SER | 77 | 2.429 | −2.045 | 9.232 | 1.00 | 0.43 | 1SG | 653 |
| ATOM | 653 | O | SER | 77 | 1.932 | −3.149 | 9.436 | 1.00 | 0.43 | 1SG | 654 |
| ATOM | 654 | N | LEU | 78 | 1.679 | −0.928 | 9.180 | 1.00 | 0.47 | 1SG | 655 |
| ATOM | 655 | CA | LEU | 78 | 0.286 | −1.025 | 9.503 | 1.00 | 0.47 | 1SG | 656 |
| ATOM | 656 | CB | LEU | 78 | −0.458 | 0.314 | 9.619 | 1.00 | 0.47 | 1SG | 657 |
| ATOM | 657 | CG | LEU | 78 | 0.194 | 1.252 | 10.645 | 1.00 | 0.47 | 1SG | 658 |
| ATOM | 658 | CD2 | LEU | 78 | 0.581 | 0.490 | 11.921 | 1.00 | 0.47 | 1SG | 659 |
| ATOM | 659 | CD1 | LEU | 78 | −0.677 | 2.490 | 10.908 | 1.00 | 0.47 | 1SG | 660 |
| ATOM | 660 | C | LEU | 78 | −0.436 | −1.804 | 8.468 | 1.00 | 0.47 | 1SG | 661 |
| ATOM | 661 | O | LEU | 78 | −1.243 | −2.669 | 8.810 | 1.00 | 0.47 | 1SG | 662 |
| ATOM | 662 | N | THR | 79 | −0.152 | −1.541 | 7.177 | 1.00 | 0.64 | 1SG | 663 |
| ATOM | 663 | CA | THR | 79 | −0.984 | −2.161 | 6.194 | 1.00 | 0.64 | 1SG | 664 |
| ATOM | 664 | CB | THR | 79 | 0.752 | −1.710 | 4.763 | 1.00 | 0.64 | 1SG | 665 |
| ATOM | 665 | OG1 | THR | 79 | −1.731 | −2.311 | 3.929 | 1.00 | 0.64 | 1SG | 666 |
| ATOM | 666 | CG2 | THR | 79 | 0.657 | −2.053 | 4.245 | 1.00 | 0.64 | 1SG | 667 |
| ATOM | 667 | C | THR | 79 | −0.925 | −3.649 | 6.274 | 1.00 | 0.64 | 1SG | 668 |
| ATOM | 668 | O | THR | 79 | −1.897 | −4.273 | 6.691 | 1.00 | 0.64 | 1SG | 669 |
| ATOM | 669 | N | VAL | 80 | 0.197 | −4.272 | 5.888 | 1.00 | 0.71 | 1SG | 670 |
| ATOM | 670 | CA | VAL | 80 | 0.150 | −5.698 | 5.853 | 1.00 | 0.71 | 1SG | 671 |
| ATOM | 671 | CB | VAL | 80 | 1.208 | −6.295 | 4.962 | 1.00 | 0.71 | 1SG | 672 |
| ATOM | 672 | CG1 | VAL | 80 | 0.902 | −5.883 | 3.513 | 1.00 | 0.71 | 1SG | 673 |
| ATOM | 673 | CG2 | VAL | 80 | 2.601 | −5.846 | 5.435 | 1.00 | 0.71 | 1SG | 674 |
| ATOM | 674 | C | VAL | 80 | 0.236 | −6.352 | 7.197 | 1.00 | 0.71 | 1SG | 675 |
| ATOM | 675 | O | VAL | 80 | −0.669 | −7.082 | 7.597 | 1.00 | 0.71 | 1SG | 676 |
| ATOM | 676 | N | LYS | 81 | 1.331 | −6.093 | 7.939 | 1.00 | 0.69 | 1SG | 677 |
| ATOM | 677 | CA | LYS | 81 | 1.587 | −6.806 | 9.159 | 1.00 | 0.69 | 1SG | 678 |
| ATOM | 678 | CB | LYS | 81 | 3.065 | −6.715 | 9.576 | 1.00 | 0.69 | 1SG | 679 |
| ATOM | 679 | CG | LYS | 81 | 3.636 | −5.297 | 9.551 | 1.00 | 0.69 | 1SG | 680 |
| ATOM | 680 | CD | LYS | 81 | 5.054 | −5.208 | 10.116 | 1.00 | 0.69 | 1SG | 681 |
| ATOM | 681 | CE | LYS | 81 | 6.103 | −5.841 | 9.197 | 1.00 | 0.69 | 1SG | 682 |
| ATOM | 682 | NZ | LYS | 81 | 7.450 | −5.762 | 9.806 | 1.00 | 0.69 | 1SG | 683 |
| ATOM | 683 | C | LYS | 81 | 0.698 | −6.397 | 10.290 | 1.00 | 0.69 | 1SG | 684 |
| ATOM | 684 | O | LYS | 81 | 0.151 | −7.243 | 10.995 | 1.00 | 0.69 | 1SG | 685 |
| ATOM | 685 | N | PHE | 82 | 0.520 | −5.082 | 10.480 | 1.00 | 0.76 | 1SG | 686 |
| ATOM | 686 | CA | PHE | 82 | −0.215 | −4.548 | 11.590 | 1.00 | 0.76 | 1SG | 687 |
| ATOM | 687 | CB | PHE | 82 | −0.159 | −3.011 | 11.616 | 1.00 | 0.76 | 1SG | 688 |
| ATOM | 688 | CG | PHE | 82 | −1.374 | −2.477 | 12.296 | 1.00 | 0.76 | 1SG | 689 |
| ATOM | 689 | CD1 | PHE | 82 | −1.429 | −2.336 | 13.661 | 1.00 | 0.76 | 1SG | 690 |
| ATOM | 690 | CD2 | PHE | 82 | −2.471 | −2.116 | 11.542 | 1.00 | 0.76 | 1SG | 691 |
| ATOM | 691 | CE1 | PHE | 82 | −2.567 | −1.843 | 14.252 | 1.00 | 0.76 | 1SG | 692 |
| ATOM | 692 | CE2 | PHE | 82 | −3.610 | −1.624 | 12.133 | 1.00 | 0.76 | 1SG | 693 |
| ATOM | 693 | CZ | PHE | 82 | −3.657 | −1.485 | 13.497 | 1.00 | 0.76 | 1SG | 694 |
| ATOM | 694 | C | PHE | 82 | −1.651 | −4.938 | 11.605 | 1.00 | 0.76 | 1SG | 695 |
| ATOM | 695 | O | PHE | 82 | −2.145 | −5.420 | 12.623 | 1.00 | 0.76 | 1SG | 696 |
| ATOM | 696 | N | LYS | 83 | −2.368 | −4.771 | 10.480 | 1.00 | 0.84 | 1SG | 697 |
| ATOM | 697 | CA | LYS | 83 | −3.786 | −4.943 | 10.591 | 1.00 | 0.84 | 1SG | 698 |
| ATOM | 698 | CB | LYS | 83 | −4.593 | −4.588 | 9.327 | 1.00 | 0.84 | 1SG | 699 |
| ATOM | 699 | CG | LYS | 83 | −4.735 | −5.739 | 8.332 | 1.00 | 0.84 | 1SG | 700 |
| ATOM | 700 | CD | LYS | 83 | −3.404 | −6.265 | 7.800 | 1.00 | 0.84 | 1SG | 701 |
| ATOM | 701 | CE | LYS | 83 | −3.550 | −7.519 | 6.939 | 1.00 | 0.84 | 1SG | 702 |
| ATOM | 702 | NZ | LYS | 83 | −4.464 | −7.258 | 5.803 | 1.00 | 0.84 | 1SG | 703 |
| ATOM | 703 | C | LYS | 83 | −4.105 | −6.351 | 10.953 | 1.00 | 0.84 | 1SG | 704 |
| ATOM | 704 | O | LYS | 83 | −3.668 | −7.307 | 10.316 | 1.00 | 0.84 | 1SG | 705 |
| ATOM | 705 | N | LYS | 84 | −4.869 | −6.506 | 12.043 | 1.00 | 0.88 | 1SG | 706 |
| ATOM | 706 | CA | LYS | 84 | −5.271 | −7.811 | 12.452 | 1.00 | 0.88 | 1SG | 707 |
| ATOM | 707 | CB | LYS | 84 | −5.987 | −7.805 | 13.811 | 1.00 | 0.88 | 1SG | 708 |
| ATOM | 708 | CG | LYS | 84 | −6.269 | −9.203 | 14.362 | 1.00 | 0.88 | 1SG | 709 |
| ATOM | 709 | CD | LYS | 84 | −5.018 | −9.977 | 14.779 | 1.00 | 0.88 | 1SG | 710 |
| ATOM | 710 | CE | LYS | 84 | −5.338 | −11.364 | 15.339 | 1.00 | 0.88 | 1SG | 711 |
| ATOM | 711 | NZ | LYS | 84 | −4.089 | −12.065 | 15.710 | 1.00 | 0.88 | 1SG | 712 |
| ATOM | 712 | C | LYS | 84 | −6.220 | −8.319 | 11.416 | 1.00 | 0.88 | 1SG | 713 |
| ATOM | 713 | O | LYS | 84 | −6.146 | −9.473 | 10.998 | 1.00 | 0.88 | 1SG | 714 |
| ATOM | 714 | N | ARG | 85 | −7.142 | −7.448 | 10.962 | 1.00 | 0.80 | 1SG | 715 |
| ATOM | 715 | CA | ARG | 85 | −8.096 | −7.895 | 9.995 | 1.00 | 0.80 | 1SG | 716 |
| ATOM | 716 | CB | ARG | 85 | −9.499 | −8.112 | 10.586 | 1.00 | 0.80 | 1SG | 717 |
| ATOM | 717 | CG | ARG | 85 | −10.536 | −8.589 | 9.570 | 1.00 | 0.80 | 1SG | 718 |
| ATOM | 718 | CD | ARG | 85 | −10.376 | −10.058 | 9.161 | 1.00 | 0.80 | 1SG | 719 |
| ATOM | 719 | NE | ARG | 85 | −10.965 | −10.885 | 10.252 | 1.00 | 0.80 | 1SG | 720 |
| ATOM | 720 | CZ | ARG | 85 | −11.497 | −12.114 | 9.983 | 1.00 | 0.80 | 1SG | 721 |
| ATOM | 721 | NH1 | ARG | 85 | −11.517 | −12.593 | 8.705 | 1.00 | 0.80 | 1SG | 722 |
| ATOM | 722 | NH2 | ARG | 85 | −12.011 | −12.863 | 11.002 | 1.00 | 0.80 | 1SG | 723 |
| ATOM | 723 | C | ARG | 85 | −8.220 | −6.849 | 8.940 | 1.00 | 0.80 | 1SG | 724 |
| ATOM | 724 | O | ARG | 85 | −8.301 | −5.654 | 9.222 | 1.00 | 0.80 | 1SG | 725 |
| ATOM | 725 | N | ALA | 86 | −8.233 | −7.295 | 7.675 | 1.00 | 0.54 | 1SG | 726 |
| ATOM | 726 | CA | ALA | 86 | −8.389 | −6.399 | 6.576 | 1.00 | 0.54 | 1SG | 727 |
| ATOM | 727 | CB | ALA | 86 | −8.258 | −7.110 | 5.218 | 1.00 | 0.54 | 1SG | 728 |
| ATOM | 728 | C | ALA | 86 | −9.768 | −5.838 | 6.668 | 1.00 | 0.54 | 1SG | 729 |
| ATOM | 729 | O | ALA | 86 | −9.990 | −4.660 | 6.401 | 1.00 | 0.54 | 1SG | 730 |
| ATOM | 730 | N | ARG | 87 | −10.733 | −6.677 | 7.087 | 1.00 | 0.47 | 1SG | 731 |
| ATOM | 731 | CA | ARG | 87 | −12.107 | −6.277 | 7.084 | 1.00 | 0.47 | 1SG | 732 |
| ATOM | 732 | CB | ARG | 87 | −13.078 | −7.473 | 7.093 | 1.00 | 0.47 | 1SG | 733 |
| ATOM | 733 | CG | ARG | 87 | −12.926 | −8.326 | 5.828 | 1.00 | 0.47 | 1SG | 734 |
| ATOM | 734 | CD | ARG | 87 | −14.025 | −9.366 | 5.594 | 1.00 | 0.47 | 1SG | 735 |
| ATOM | 735 | NE | ARG | 87 | −13.710 | −10.036 | 4.297 | 1.00 | 0.47 | 1SG | 736 |
| ATOM | 736 | CZ | ARG | 87 | −14.699 | −10.611 | 3.550 | 1.00 | 0.47 | 1SG | 737 |
| ATOM | 737 | NH1 | ARG | 87 | −15.989 | −10.601 | 3.995 | 1.00 | 0.47 | 1SG | 738 |
| ATOM | 738 | NH2 | ARG | 87 | −14.399 | −11.192 | 2.351 | 1.00 | 0.47 | 1SG | 739 |
| ATOM | 739 | C | ARG | 87 | −12.431 | −5.358 | 8.225 | 1.00 | 0.47 | 1SG | 740 |
| ATOM | 740 | O | ARG | 87 | −11.554 | −4.788 | 8.873 | 1.00 | 0.47 | 1SG | 741 |
| ATOM | 741 | N | TRP | 88 | −13.748 | −5.216 | 8.478 | 1.00 | 0.66 | 1SG | 742 |
| ATOM | 742 | CA | TRP | 88 | −14.366 | −4.289 | 9.391 | 1.00 | 0.66 | 1SG | 743 |
| ATOM | 743 | CB | TRP | 88 | −15.895 | −4.455 | 9.446 | 1.00 | 0.66 | 1SG | 744 |
| ATOM | 744 | CG | TRP | 88 | −16.627 | −4.224 | 8.147 | 1.00 | 0.66 | 1SG | 745 |
| ATOM | 745 | CD2 | TRP | 88 | −18.008 | −4.559 | 7.941 | 1.00 | 0.66 | 1SG | 746 |
| ATOM | 746 | CD1 | TRP | 88 | −16.171 | −3.720 | 6.963 | 1.00 | 0.66 | 1SG | 747 |
| ATOM | 747 | NE1 | TRP | 88 | −17.181 | −3.725 | 6.031 | 1.00 | 0.66 | 1SG | 748 |
| ATOM | 748 | CE2 | TRP | 88 | −18.316 | −4.239 | 6.617 | 1.00 | 0.66 | 1SG | 749 |
| ATOM | 749 | CE3 | TRP | 88 | −18.940 | −5.098 | 8.783 | 1.00 | 0.66 | 1SG | 750 |
| ATOM | 750 | CZ2 | TRP | 88 | −19.564 | −4.450 | 6.108 | 1.00 | 0.66 | 1SG | 751 |
| ATOM | 751 | CZ3 | TRP | 88 | −20.199 | −5.303 | 8.264 | 1.00 | 0.66 | 1SG | 752 |
| ATOM | 752 | CH2 | TRP | 88 | −20.505 | −4.987 | 6.955 | 1.00 | 0.66 | 1SG | 753 |
| ATOM | 753 | C | TRP | 88 | −13.929 | −4.528 | 10.795 | 1.00 | 0.66 | 1SG | 754 |
| ATOM | 754 | O | TRP | 88 | −13.699 | −3.571 | 11.536 | 1.00 | 0.66 | 1SG | 755 |
| ATOM | 755 | N | THR | 89 | −13.820 | −5.812 | 11.190 | 1.00 | 0.71 | 1SG | 756 |
| ATOM | 756 | CA | THR | 89 | −13.526 | −6.156 | 12.550 | 1.00 | 0.71 | 1SG | 757 |
| ATOM | 757 | CB | THR | 89 | −13.341 | −7.630 | 12.765 | 1.00 | 0.71 | 1SG | 758 |
| ATOM | 758 | OG1 | THR | 89 | −13.329 | −7.921 | 14.154 | 1.00 | 0.71 | 1SG | 759 |
| ATOM | 759 | CG2 | THR | 89 | −12.023 | −8.075 | 12.116 | 1.00 | 0.71 | 1SG | 760 |
| ATOM | 760 | C | THR | 89 | −12.282 | −5.437 | 12.945 | 1.00 | 0.71 | 1SG | 761 |
| ATOM | 761 | O | THR | 89 | −12.150 | −4.979 | 14.078 | 1.00 | 0.71 | 1SG | 762 |
| ATOM | 762 | N | ASN | 90 | −11.331 | −5.311 | 12.007 | 1.00 | 0.54 | 1SG | 763 |
| ATOM | 763 | CA | ASN | 90 | −10.164 | −4.532 | 12.282 | 1.00 | 0.54 | 1SG | 764 |
| ATOM | 764 | CB | ASN | 90 | −8.849 | −5.273 | 12.012 | 1.00 | 0.54 | 1SG | 765 |
| ATOM | 765 | CG | ASN | 90 | −8.641 | −6.184 | 13.217 | 1.00 | 0.54 | 1SG | 766 |
| ATOM | 766 | OD1 | ASN | 90 | −8.628 | −7.410 | 13.123 | 1.00 | 0.54 | 1SG | 767 |
| ATOM | 767 | ND2 | ASN | 90 | −8.490 | −5.553 | 14.412 | 1.00 | 0.54 | 1SG | 768 |
| ATOM | 768 | C | ASN | 90 | −10.256 | −3.337 | 11.397 | 1.00 | 0.54 | 1SG | 769 |
| ATOM | 769 | O | ASN | 90 | −11.325 | −2.754 | 11.247 | 1.00 | 0.54 | 1SG | 770 |
| ATOM | 770 | N | TYR | 91 | −9.142 | −2.902 | 10.792 | 1.00 | 0.40 | 1SG | 771 |
| ATOM | 771 | CA | TYR | 91 | −9.302 | −1.764 | 9.944 | 1.00 | 0.40 | 1SG | 772 |
| ATOM | 772 | CB | TYR | 91 | −7.992 | −0.998 | 9.707 | 1.00 | 0.40 | 1SG | 773 |
| ATOM | 773 | CG | TYR | 91 | −7.623 | −0.395 | 11.018 | 1.00 | 0.40 | 1SG | 774 |
| ATOM | 774 | CD1 | TYR | 91 | −8.126 | 0.828 | 11.388 | 1.00 | 0.40 | 1SG | 775 |
| ATOM | 775 | CD2 | TYR | 91 | −6.779 | −1.058 | 11.880 | 1.00 | 0.40 | 1SG | 776 |
| ATOM | 776 | CE1 | TYR | 91 | −7.785 | 1.379 | 12.599 | 1.00 | 0.40 | 1SG | 777 |
| ATOM | 777 | CE2 | TYR | 91 | −6.437 | −0.507 | 13.093 | 1.00 | 0.40 | 1SG | 778 |
| ATOM | 778 | CZ | TYR | 91 | −6.945 | 0.713 | 13.458 | 1.00 | 0.40 | 1SG | 779 |
| ATOM | 779 | OH | TYR | 91 | −6.604 | 1.285 | 14.701 | 1.00 | 0.40 | 1SG | 780 |
| ATOM | 780 | C | TYR | 91 | −9.795 | −2.285 | 8.643 | 1.00 | 0.40 | 1SG | 781 |
| ATOM | 781 | O | TYR | 91 | −9.016 | −2.823 | 7.857 | 1.00 | 0.40 | 1SG | 782 |
| ATOM | 782 | N | ASP | 92 | −11.110 | −2.109 | 8.386 | 1.00 | 0.53 | 1SG | 783 |
| ATOM | 783 | CA | ASP | 92 | −11.707 | −2.625 | 7.191 | 1.00 | 0.53 | 1SG | 784 |
| ATOM | 784 | CB | ASP | 92 | −13.167 | −2.204 | 6.971 | 1.00 | 0.53 | 1SG | 785 |
| ATOM | 785 | CG | ASP | 92 | −13.634 | −2.844 | 5.669 | 1.00 | 0.53 | 1SG | 786 |
| ATOM | 786 | OD1 | ASP | 92 | −13.022 | −3.864 | 5.253 | 1.00 | 0.53 | 1SG | 787 |
| ATOM | 787 | OD2 | ASP | 92 | −14.612 | −2.320 | 5.070 | 1.00 | 0.53 | 1SG | 788 |
| ATOM | 788 | C | ASP | 92 | −10.939 | −2.050 | 6.071 | 1.00 | 0.53 | 1SG | 789 |
| ATOM | 789 | O | ASP | 92 | −10.757 | −2.679 | 5.029 | 1.00 | 0.53 | 1SG | 790 |
| ATOM | 790 | N | TYR | 93 | −10.470 | −0.806 | 6.232 | 1.00 | 0.57 | 1SG | 791 |
| ATOM | 791 | CA | TYR | 93 | −9.683 | −0.408 | 5.126 | 1.00 | 0.57 | 1SG | 792 |
| ATOM | 792 | CB | TYR | 93 | −10.513 | 0.163 | 3.968 | 1.00 | 0.57 | 1SG | 793 |
| ATOM | 793 | CG | TYR | 93 | −9.712 | −0.120 | 2.751 | 1.00 | 0.57 | 1SG | 794 |
| ATOM | 794 | CD1 | TYR | 93 | −8.695 | 0.705 | 2.351 | 1.00 | 0.57 | 1SG | 795 |
| ATOM | 795 | CD2 | TYR | 93 | −9.970 | −1.267 | 2.035 | 1.00 | 0.57 | 1SG | 796 |
| ATOM | 796 | CE1 | TYR | 93 | −7.979 | 0.401 | 1.220 | 1.00 | 0.57 | 1SG | 797 |
| ATOM | 797 | CE2 | TYR | 93 | −9.257 | −1.575 | 0.904 | 1.00 | 0.57 | 1SG | 798 |
| ATOM | 798 | CZ | TYR | 93 | −8.243 | −0.742 | 0.504 | 1.00 | 0.57 | 1SG | 799 |
| ATOM | 799 | OH | TYR | 93 | −7.495 | −1.041 | −0.652 | 1.00 | 0.57 | 1SG | 800 |
| ATOM | 800 | C | TYR | 93 | −8.745 | 0.655 | 5.585 | 1.00 | 0.57 | 1SG | 801 |
| ATOM | 801 | O | TYR | 93 | −9.044 | 1.418 | 6.504 | 1.00 | 0.57 | 1SG | 802 |
| ATOM | 802 | N | ILE | 94 | −7.559 | 0.705 | 4.952 | 1.00 | 0.30 | 1SG | 803 |
| ATOM | 803 | CA | ILE | 94 | −6.622 | 1.741 | 5.251 | 1.00 | 0.30 | 1SG | 804 |
| ATOM | 804 | CB | ILE | 94 | −5.184 | 1.318 | 5.134 | 1.00 | 0.30 | 1SG | 805 |
| ATOM | 805 | CG2 | ILE | 94 | −4.306 | 2.572 | 5.281 | 1.00 | 0.30 | 1SG | 806 |
| ATOM | 806 | CG1 | ILE | 94 | −4.850 | 0.205 | 6.141 | 1.00 | 0.30 | 1SG | 807 |
| ATOM | 807 | CD1 | ILE | 94 | −3.497 | −0.459 | 5.884 | 1.00 | 0.30 | 1SG | 808 |
| ATOM | 808 | C | ILE | 94 | −6.849 | 2.759 | 4.190 | 1.00 | 0.30 | 1SG | 809 |
| ATOM | 809 | O | ILE | 94 | −6.697 | 2.476 | 3.002 | 1.00 | 0.30 | 1SG | 810 |
| ATOM | 810 | N | ASN | 95 | −7.257 | 3.975 | 4.588 | 1.00 | 0.29 | 1SG | 811 |
| ATOM | 811 | CA | ASN | 95 | −7.501 | 4.974 | 3.596 | 1.00 | 0.29 | 1SG | 812 |
| ATOM | 812 | CB | ASN | 95 | −8.848 | 5.691 | 3.787 | 1.00 | 0.29 | 1SG | 813 |
| ATOM | 813 | CG | ASN | 95 | −9.938 | 4.657 | 3.537 | 1.00 | 0.29 | 1SG | 814 |
| ATOM | 814 | OD1 | ASN | 95 | −9.922 | 3.959 | 2.524 | 1.00 | 0.29 | 1SG | 815 |
| ATOM | 815 | ND2 | ASN | 95 | −10.901 | 4.537 | 4.490 | 1.00 | 0.29 | 1SG | 816 |
| ATOM | 816 | C | ASN | 95 | −6.406 | 5.978 | 3.710 | 1.00 | 0.29 | 1SG | 817 |
| ATOM | 817 | O | ASN | 95 | −6.261 | 6.648 | 4.732 | 1.00 | 0.29 | 1SG | 818 |
| ATOM | 818 | N | MET | 96 | −5.592 | 6.096 | 2.643 | 1.00 | 0.47 | 1SG | 819 |
| ATOM | 819 | CA | MET | 96 | −4.509 | 7.028 | 2.674 | 1.00 | 0.47 | 1SG | 820 |
| ATOM | 820 | CB | MET | 96 | −3.208 | 6.505 | 2.030 | 1.00 | 0.47 | 1SG | 821 |
| ATOM | 821 | CG | MET | 96 | −3.313 | 6.252 | 0.520 | 1.00 | 0.47 | 1SG | 822 |
| ATOM | 822 | SD | MET | 96 | −1.763 | 5.793 | −0.313 | 1.00 | 0.47 | 1SG | 823 |
| ATOM | 823 | CE | MET | 96 | −1.073 | 7.471 | −0.342 | 1.00 | 0.47 | 1SG | 824 |
| ATOM | 824 | C | MET | 96 | −4.925 | 8.230 | 1.901 | 1.00 | 0.47 | 1SG | 825 |
| ATOM | 825 | O | MET | 96 | −5.271 | 8.151 | 0.723 | 1.00 | 0.47 | 1SG | 826 |
| ATOM | 826 | N | THR | 97 | −4.933 | 9.397 | 2.558 | 1.00 | 0.31 | 1SG | 827 |
| ATOM | 827 | CA | THR | 97 | −5.245 | 10.563 | 1.805 | 1.00 | 0.31 | 1SG | 828 |
| ATOM | 828 | CB | THR | 97 | −6.300 | 11.432 | 2.413 | 1.00 | 0.31 | 1SG | 829 |
| ATOM | 829 | OG1 | THR | 97 | −7.523 | 10.723 | 2.521 | 1.00 | 0.31 | 1SG | 830 |
| ATOM | 830 | CG2 | THR | 97 | −6.479 | 12.696 | 1.553 | 1.00 | 0.31 | 1SG | 831 |
| ATOM | 831 | C | THR | 97 | −3.986 | 11.338 | 1.741 | 1.00 | 0.31 | 1SG | 832 |
| ATOM | 832 | O | THR | 97 | −3.334 | 11.584 | 2.754 | 1.00 | 0.31 | 1SG | 833 |
| ATOM | 833 | N | GLY | 98 | −3.596 | 11.747 | 0.527 | 1.00 | 0.29 | 1SG | 834 |
| ATOM | 834 | CA | GLY | 98 | −2.370 | 12.467 | 0.468 | 1.00 | 0.29 | 1SG | 835 |
| ATOM | 835 | C | GLY | 98 | −2.248 | 13.055 | −0.883 | 1.00 | 0.29 | 1SG | 836 |
| ATOM | 836 | O | GLY | 98 | −3.104 | 12.877 | −1.748 | 1.00 | 0.29 | 1SG | 837 |
| ATOM | 837 | N | ASP | 99 | −1.159 | 13.812 | −1.070 | 1.00 | 0.75 | 1SG | 838 |
| ATOM | 838 | CA | ASP | 99 | −0.892 | 14.412 | −2.334 | 1.00 | 0.75 | 1SG | 839 |
| ATOM | 839 | CB | ASP | 99 | 0.002 | 15.658 | −2.234 | 1.00 | 0.75 | 1SG | 840 |
| ATOM | 840 | CG | ASP | 99 | −0.809 | 16.777 | −1.612 | 1.00 | 0.75 | 1SG | 841 |
| ATOM | 841 | OD1 | ASP | 99 | −2.022 | 16.862 | −1.933 | 1.00 | 0.75 | 1SG | 842 |
| ATOM | 842 | OD2 | ASP | 99 | −0.234 | 17.555 | −0.805 | 1.00 | 0.75 | 1SG | 843 |
| ATOM | 843 | C | ASP | 99 | −0.138 | 13.408 | −3.148 | 1.00 | 0.75 | 1SG | 844 |
| ATOM | 844 | O | ASP | 99 | 0.085 | 13.607 | −4.339 | 1.00 | 0.75 | 1SG | 845 |
| ATOM | 845 | N | CYS | 100 | 0.268 | 12.288 | −2.521 | 1.00 | 1.13 | 1SG | 846 |
| ATOM | 846 | CA | CYS | 100 | 1.111 | 11.334 | −3.187 | 1.00 | 1.13 | 1SG | 847 |
| ATOM | 847 | CB | CYS | 100 | 2.366 | 10.981 | −2.368 | 1.00 | 1.13 | 1SG | 848 |
| ATOM | 848 | SG | CYS | 100 | 3.565 | 12.333 | −2.122 | 1.00 | 1.13 | 1SG | 849 |
| ATOM | 849 | C | CYS | 100 | 0.350 | 10.043 | −3.364 | 1.00 | 1.13 | 1SG | 850 |
| ATOM | 850 | O | CYS | 100 | −0.870 | 10.008 | −3.212 | 1.00 | 1.13 | 1SG | 851 |
| ATOM | 851 | N | ALA | 101 | 1.067 | 8.947 | −3.727 | 1.00 | 1.07 | 1SG | 852 |
| ATOM | 852 | CA | ALA | 101 | 0.479 | 7.646 | −3.935 | 1.00 | 1.07 | 1SG | 853 |
| ATOM | 853 | CB | ALA | 101 | 0.398 | 7.244 | −5.418 | 1.00 | 1.07 | 1SG | 854 |
| ATOM | 854 | C | ALA | 101 | 1.352 | 6.632 | −3.250 | 1.00 | 1.07 | 1SG | 855 |
| ATOM | 855 | O | ALA | 101 | 2.089 | 6.965 | −2.324 | 1.00 | 1.07 | 1SG | 856 |
| ATOM | 856 | N | SER | 102 | 1.274 | 5.349 | −3.672 | 1.00 | 1.01 | 1SG | 857 |
| ATOM | 857 | CA | SER | 102 | 2.097 | 4.339 | −3.060 | 1.00 | 1.01 | 1SG | 858 |
| ATOM | 858 | CB | SER | 102 | 2.030 | 4.345 | −1.521 | 1.00 | 1.01 | 1SG | 859 |
| ATOM | 859 | OG | SER | 102 | 0.705 | 4.072 | −1.087 | 1.00 | 1.01 | 1SG | 860 |
| ATOM | 860 | C | SER | 102 | 1.623 | 2.993 | −3.513 | 1.00 | 1.01 | 1SG | 861 |
| ATOM | 861 | O | SER | 102 | 1.328 | 2.787 | −4.689 | 1.00 | 1.01 | 1SG | 862 |
| ATOM | 862 | N | PHE | 103 | 1.565 | 2.032 | −2.565 | 1.00 | 1.14 | 1SG | 863 |
| ATOM | 863 | CA | PHE | 103 | 1.117 | 0.697 | −2.847 | 1.00 | 1.14 | 1SG | 864 |
| ATOM | 864 | CB | PHE | 103 | 1.258 | −0.258 | −1.651 | 1.00 | 1.14 | 1SG | 865 |
| ATOM | 865 | CG | PHE | 103 | 2.702 | −0.332 | −1.298 | 1.00 | 1.14 | 1SG | 866 |
| ATOM | 866 | CD1 | PHE | 103 | 3.561 | −1.125 | −2.022 | 1.00 | 1.14 | 1SG | 867 |
| ATOM | 867 | CD2 | PHE | 103 | 3.196 | 0.390 | −0.236 | 1.00 | 1.14 | 1SG | 868 |
| ATOM | 868 | CE1 | PHE | 103 | 4.894 | −1.192 | −1.694 | 1.00 | 1.14 | 1SG | 869 |
| ATOM | 869 | CE2 | PHE | 103 | 4.528 | 0.325 | 0.097 | 1.00 | 1.14 | 1SG | 870 |
| ATOM | 870 | CZ | PHE | 103 | 5.380 | −0.467 | −0.633 | 1.00 | 1.14 | 1SG | 871 |
| ATOM | 871 | C | PHE | 103 | −0.339 | 0.802 | −3.140 | 1.00 | 1.14 | 1SG | 872 |
| ATOM | 872 | O | PHE | 103 | −1.127 | 1.160 | −2.270 | 1.00 | 1.14 | 1SG | 873 |
| ATOM | 873 | N | ILE | 104 | −0.736 | 0.487 | −4.384 | 1.00 | 1.10 | 1SG | 874 |
| ATOM | 874 | CA | ILE | 104 | −2.102 | 0.660 | −4.778 | 1.00 | 1.10 | 1SG | 875 |
| ATOM | 875 | CB | ILE | 104 | −2.278 | 0.554 | −6.279 | 1.00 | 1.10 | 1SG | 876 |
| ATOM | 876 | CG2 | ILE | 104 | −1.954 | −0.882 | −6.723 | 1.00 | 1.10 | 1SG | 877 |
| ATOM | 877 | CG1 | ILE | 104 | −3.656 | 1.070 | −6.743 | 1.00 | 1.10 | 1SG | 878 |
| ATOM | 878 | CD1 | ILE | 104 | −4.855 | 0.233 | −6.296 | 1.00 | 1.10 | 1SG | 879 |
| ATOM | 879 | C | ILE | 104 | −3.008 | −0.314 | −4.076 | 1.00 | 1.10 | 1SG | 880 |
| ATOM | 880 | O | ILE | 104 | −4.002 | 0.087 | −3.475 | 1.00 | 1.10 | 1SG | 881 |
| ATOM | 881 | N | LYS | 105 | −2.675 | −1.616 | −4.099 | 1.00 | 0.82 | 1SG | 882 |
| ATOM | 882 | CA | LYS | 105 | −3.562 | −2.638 | −3.611 | 1.00 | 0.82 | 1SG | 883 |
| ATOM | 883 | CB | LYS | 105 | −3.017 | −4.052 | −3.875 | 1.00 | 0.82 | 1SG | 884 |
| ATOM | 884 | CG | LYS | 105 | −3.978 | −5.174 | −3.474 | 1.00 | 0.82 | 1SG | 885 |
| ATOM | 885 | CD | LYS | 105 | −5.232 | −5.250 | −4.348 | 1.00 | 0.82 | 1SG | 886 |
| ATOM | 886 | CE | LYS | 105 | −6.193 | −6.370 | −3.945 | 1.00 | 0.82 | 1SG | 887 |
| ATOM | 887 | NZ | LYS | 105 | −7.373 | −6.372 | −4.839 | 1.00 | 0.82 | 1SG | 888 |
| ATOM | 888 | C | LYS | 105 | −3.814 | −2.533 | −2.138 | 1.00 | 0.82 | 1SG | 889 |
| ATOM | 889 | O | LYS | 105 | −4.952 | −2.661 | −1.688 | 1.00 | 0.82 | 1SG | 890 |
| ATOM | 890 | N | LYS | 106 | −2.764 | −2.272 | −1.345 | 1.00 | 0.54 | 1SG | 891 |
| ATOM | 891 | CA | LYS | 106 | −2.898 | −2.340 | 0.080 | 1.00 | 0.54 | 1SG | 892 |
| ATOM | 892 | CB | LYS | 106 | −1.564 | −2.085 | 0.799 | 1.00 | 0.54 | 1SG | 893 |
| ATOM | 893 | CG | LYS | 106 | −0.543 | −3.201 | 0.562 | 1.00 | 0.54 | 1SG | 894 |
| ATOM | 894 | CD | LYS | 106 | −0.077 | −3.314 | −0.891 | 1.00 | 0.54 | 1SG | 895 |
| ATOM | 895 | CE | LYS | 106 | 0.944 | −4.431 | −1.125 | 1.00 | 0.54 | 1SG | 896 |
| ATOM | 896 | NZ | LYS | 106 | 1.332 | −4.473 | −2.553 | 1.00 | 0.54 | 1SG | 897 |
| ATOM | 897 | C | LYS | 106 | −3.899 | −1.348 | 0.581 | 1.00 | 0.54 | 1SG | 898 |
| ATOM | 898 | O | LYS | 106 | −4.687 | −1.654 | 1.477 | 1.00 | 0.54 | 1SG | 899 |
| ATOM | 899 | N | ARG | 107 | −3.919 | −0.131 | 0.015 | 1.00 | 0.46 | 1SG | 900 |
| ATOM | 900 | CA | ARG | 107 | −4.794 | 0.847 | 0.589 | 1.00 | 0.46 | 1SG | 901 |
| ATOM | 901 | CB | ARG | 107 | −4.006 | 1.905 | 1.373 | 1.00 | 0.46 | 1SG | 902 |
| ATOM | 902 | CG | ARG | 107 | −3.018 | 2.672 | 0.491 | 1.00 | 0.46 | 1SG | 903 |
| ATOM | 903 | CD | ARG | 107 | −1.762 | 3.138 | 1.229 | 1.00 | 0.46 | 1SG | 904 |
| ATOM | 904 | NE | ARG | 107 | −0.727 | 2.079 | 1.044 | 1.00 | 0.46 | 1SG | 905 |
| ATOM | 905 | CZ | ARG | 107 | −0.670 | 1.009 | 1.889 | 1.00 | 0.46 | 1SG | 906 |
| ATOM | 906 | NH1 | ARG | 107 | −1.560 | 0.903 | 2.918 | 1.00 | 0.46 | 1SG | 907 |
| ATOM | 907 | NH2 | ARG | 107 | 0.275 | 0.042 | 1.700 | 1.00 | 0.46 | 1SG | 908 |
| ATOM | 908 | C | ARG | 107 | −5.564 | 1.549 | −0.487 | 1.00 | 0.46 | 1SG | 909 |
| ATOM | 909 | O | ARG | 107 | −5.363 | 1.311 | −1.677 | 1.00 | 0.46 | 1SG | 910 |
| ATOM | 910 | N | LYS | 108 | −6.505 | 2.420 | −0.059 | 1.00 | 0.60 | 1SG | 911 |
| ATOM | 911 | CA | LYS | 108 | −7.313 | 3.205 | −0.947 | 1.00 | 0.60 | 1SG | 912 |
| ATOM | 912 | CB | LYS | 108 | −8.758 | 3.422 | −0.456 | 1.00 | 0.60 | 1SG | 913 |
| ATOM | 913 | CG | LYS | 108 | −9.637 | 2.169 | −0.449 | 1.00 | 0.60 | 1SG | 914 |
| ATOM | 914 | CD | LYS | 108 | −9.896 | 1.574 | −1.834 | 1.00 | 0.60 | 1SG | 915 |
| ATOM | 915 | CE | LYS | 108 | −10.778 | 0.323 | −1.798 | 1.00 | 0.60 | 1SG | 916 |
| ATOM | 916 | NZ | LYS | 108 | −11.015 | −0.173 | −3.171 | 1.00 | 0.60 | 1SG | 917 |
| ATOM | 917 | C | LYS | 108 | −6.677 | 4.552 | −0.970 | 1.00 | 0.60 | 1SG | 918 |
| ATOM | 918 | O | LYS | 108 | −6.381 | 5.124 | 0.079 | 1.00 | 0.60 | 1SG | 919 |
| ATOM | 919 | N | TYR | 109 | −6.452 | 5.105 | −2.174 | 1.00 | 0.85 | 1SG | 920 |
| ATOM | 920 | CA | TYR | 109 | −5.752 | 6.349 | −2.211 | 1.00 | 0.85 | 1SG | 921 |
| ATOM | 921 | CB | TYR | 109 | −4.559 | 6.356 | −3.187 | 1.00 | 0.85 | 1SG | 922 |
| ATOM | 922 | CG | TYR | 109 | −5.075 | 6.176 | −4.575 | 1.00 | 0.85 | 1SG | 923 |
| ATOM | 923 | CD1 | TYR | 109 | −5.456 | 7.265 | −5.326 | 1.00 | 0.85 | 1SG | 924 |
| ATOM | 924 | CD2 | TYR | 109 | −5.177 | 4.920 | −5.127 | 1.00 | 0.85 | 1SG | 925 |
| ATOM | 925 | CE1 | TYR | 109 | −5.930 | 7.103 | −6.607 | 1.00 | 0.85 | 1SG | 926 |
| ATOM | 926 | CE2 | TYR | 109 | −5.651 | 4.751 | −6.407 | 1.00 | 0.85 | 1SG | 927 |
| ATOM | 927 | CZ | TYR | 109 | −6.028 | 5.844 | −7.149 | 1.00 | 0.85 | 1SG | 928 |
| ATOM | 928 | OH | TYR | 109 | −6.514 | 5.675 | −8.463 | 1.00 | 0.85 | 1SG | 929 |
| ATOM | 929 | C | TYR | 109 | −6.683 | 7.437 | −2.620 | 1.00 | 0.85 | 1SG | 930 |
| ATOM | 930 | O | TYR | 109 | −7.515 | 7.277 | −3.513 | 1.00 | 0.85 | 1SG | 931 |
| ATOM | 931 | N | ILE | 110 | −6.570 | 8.580 | −1.923 | 1.00 | 0.95 | 1SG | 932 |
| ATOM | 932 | CA | ILE | 110 | −7.337 | 9.740 | −2.250 | 1.00 | 0.95 | 1SG | 933 |
| ATOM | 933 | CB | ILE | 110 | −8.349 | 10.110 | −1.209 | 1.00 | 0.95 | 1SG | 934 |
| ATOM | 934 | CG2 | ILE | 110 | −9.028 | 11.400 | −1.684 | 1.00 | 0.95 | 1SG | 935 |
| ATOM | 935 | CG1 | ILE | 110 | −9.337 | 8.960 | −0.958 | 1.00 | 0.95 | 1SG | 936 |
| ATOM | 936 | CD1 | ILE | 110 | −8.705 | 7.755 | −0.263 | 1.00 | 0.95 | 1SG | 937 |
| ATOM | 937 | C | ILE | 110 | −6.344 | 10.853 | −2.273 | 1.00 | 0.95 | 1SG | 938 |
| ATOM | 938 | O | ILE | 110 | −5.402 | 10.861 | −1.483 | 1.00 | 0.95 | 1SG | 939 |
| ATOM | 939 | N | THR | 111 | −6.505 | 11.821 | −3.193 | 1.00 | 0.86 | 1SG | 940 |
| ATOM | 940 | CA | THR | 111 | −5.550 | 12.887 | −3.206 | 1.00 | 0.86 | 1SG | 941 |
| ATOM | 941 | CB | THR | 111 | −4.696 | 12.922 | −4.439 | 1.00 | 0.86 | 1SG | 942 |
| ATOM | 942 | OG1 | THR | 111 | −3.657 | 13.878 | −4.287 | 1.00 | 0.86 | 1SG | 943 |
| ATOM | 943 | CG2 | THR | 111 | −5.579 | 13.279 | −5.647 | 1.00 | 0.86 | 1SG | 944 |
| ATOM | 944 | C | THR | 111 | −6.294 | 14.177 | −3.140 | 1.00 | 0.86 | 1SG | 945 |
| ATOM | 945 | O | THR | 111 | −7.383 | 14.309 | −3.695 | 1.00 | 0.86 | 1SG | 946 |
| ATOM | 946 | N | GLU | 112 | −5.722 | 15.170 | −2.432 | 1.00 | 0.91 | 1SG | 947 |
| ATOM | 947 | CA | GLU | 112 | −6.381 | 16.439 | −2.371 | 1.00 | 0.91 | 1SG | 948 |
| ATOM | 948 | CB | GLU | 112 | −5.915 | 17.377 | −1.244 | 1.00 | 0.91 | 1SG | 949 |
| ATOM | 949 | CG | GLU | 112 | −4.504 | 17.916 | −1.472 | 1.00 | 0.91 | 1SG | 950 |
| ATOM | 950 | CD | GLU | 112 | −4.261 | 19.110 | −0.559 | 1.00 | 0.91 | 1SG | 951 |
| ATOM | 951 | OE1 | GLU | 112 | −4.501 | 18.984 | 0.671 | 1.00 | 0.91 | 1SG | 952 |
| ATOM | 952 | OE2 | GLU | 112 | −3.831 | 20.171 | −1.088 | 1.00 | 0.91 | 1SG | 953 |
| ATOM | 953 | C | GLU | 112 | −6.039 | 17.134 | −3.646 | 1.00 | 0.91 | 1SG | 954 |
| ATOM | 954 | O | GLU | 112 | −5.383 | 16.572 | −4.522 | 1.00 | 0.91 | 1SG | 955 |
| ATOM | 955 | N | PRO | 113 | −6.487 | 18.349 | −3.769 | 1.00 | 1.07 | 1SG | 956 |
| ATOM | 956 | CA | PRO | 113 | −6.199 | 19.065 | −4.981 | 1.00 | 1.07 | 1SG | 957 |
| ATOM | 957 | CD | PRO | 113 | −7.809 | 18.670 | −3.257 | 1.00 | 1.07 | 1SG | 958 |
| ATOM | 958 | CB | PRO | 113 | −7.262 | 20.155 | −5.096 | 1.00 | 1.07 | 1SG | 959 |
| ATOM | 959 | CG | PRO | 113 | −8.455 | 19.579 | −4.314 | 1.00 | 1.07 | 1SG | 960 |
| ATOM | 960 | C | PRO | 113 | −4.799 | 19.595 | −4.996 | 1.00 | 1.07 | 1SG | 961 |
| ATOM | 961 | O | PRO | 113 | −4.248 | 19.885 | −3.935 | 1.00 | 1.07 | 1SG | 962 |
| ATOM | 962 | N | LEU | 114 | −4.212 | 19.704 | −6.204 | 1.00 | 0.85 | 1SG | 963 |
| ATOM | 963 | CA | LEU | 114 | −2.871 | 20.163 | −6.427 | 1.00 | 0.85 | 1SG | 964 |
| ATOM | 964 | CB | LEU | 114 | −2.404 | 19.929 | −7.878 | 1.00 | 0.85 | 1SG | 965 |
| ATOM | 965 | CG | LEU | 114 | −0.969 | 20.404 | −8.197 | 1.00 | 0.85 | 1SG | 966 |
| ATOM | 966 | CD2 | LEU | 114 | 0.027 | 19.845 | −7.171 | 1.00 | 0.85 | 1SG | 967 |
| ATOM | 967 | CD1 | LEU | 114 | −0.863 | 21.931 | −8.351 | 1.00 | 0.85 | 1SG | 968 |
| ATOM | 968 | C | LEU | 114 | −2.772 | 21.633 | −6.134 | 1.00 | 0.85 | 1SG | 969 |
| ATOM | 969 | O | LEU | 114 | −1.737 | 22.077 | −5.638 | 1.00 | 0.85 | 1SG | 970 |
| ATOM | 970 | N | SER | 115 | −3.844 | 22.390 | −6.446 | 1.00 | 0.36 | 1SG | 971 |
| ATOM | 971 | CA | SER | 115 | −3.869 | 23.828 | −6.365 | 1.00 | 0.36 | 1SG | 972 |
| ATOM | 972 | CB | SER | 115 | −5.288 | 24.421 | −6.410 | 1.00 | 0.36 | 1SG | 973 |
| ATOM | 973 | OG | SER | 115 | −6.016 | 24.030 | −5.254 | 1.00 | 0.36 | 1SG | 974 |
| ATOM | 974 | C | SER | 115 | −3.212 | 24.308 | −5.113 | 1.00 | 0.36 | 1SG | 975 |
| ATOM | 975 | O | SER | 115 | −3.489 | 23.832 | −4.013 | 1.00 | 0.36 | 1SG | 976 |
| ATOM | 976 | N | LYS | 116 | −2.290 | 25.275 | −5.283 | 1.00 | 0.21 | 1SG | 977 |
| ATOM | 977 | CA | LYS | 116 | −1.554 | 25.825 | −4.188 | 1.00 | 0.21 | 1SG | 978 |
| ATOM | 978 | CB | LYS | 116 | −0.169 | 26.354 | −4.601 | 1.00 | 0.21 | 1SG | 979 |
| ATOM | 979 | CG | LYS | 116 | 0.759 | 25.252 | −5.120 | 1.00 | 0.21 | 1SG | 980 |
| ATOM | 980 | CD | LYS | 116 | 1.989 | 25.774 | −5.865 | 1.00 | 0.21 | 1SG | 981 |
| ATOM | 981 | CE | LYS | 116 | 2.907 | 24.662 | −6.378 | 1.00 | 0.21 | 1SG | 982 |
| ATOM | 982 | NZ | LYS | 116 | 4.042 | 25.244 | −7.129 | 1.00 | 0.21 | 1SG | 983 |
| ATOM | 983 | C | LYS | 116 | −2.340 | 26.969 | −3.644 | 1.00 | 0.21 | 1SG | 984 |
| ATOM | 984 | O | LYS | 116 | −3.291 | 27.438 | −4.268 | 1.00 | 0.21 | 1SG | 985 |
| ATOM | 985 | N | GLU | 117 | −1.966 | 27.437 | −2.438 | 1.00 | 0.47 | 1SG | 986 |
| ATOM | 986 | CA | GLU | 117 | −2.667 | 28.531 | −1.841 | 1.00 | 0.47 | 1SG | 987 |
| ATOM | 987 | CB | GLU | 117 | −3.248 | 28.210 | −0.453 | 1.00 | 0.47 | 1SG | 988 |
| ATOM | 988 | CG | GLU | 117 | −4.415 | 27.222 | −0.498 | 1.00 | 0.47 | 1SG | 989 |
| ATOM | 989 | CD | GLU | 117 | −3.862 | 25.857 | −0.877 | 1.00 | 0.47 | 1SG | 990 |
| ATOM | 990 | OE1 | GLU | 117 | −2.855 | 25.431 | −0.251 | 1.00 | 0.47 | 1SG | 991 |
| ATOM | 991 | OE2 | GLU | 117 | −4.436 | 25.224 | −1.804 | 1.00 | 0.47 | 1SG | 992 |
| ATOM | 992 | C | GLU | 117 | −1.697 | 29.652 | −1.671 | 1.00 | 0.47 | 1SG | 993 |
| ATOM | 993 | O | GLU | 117 | −0.497 | 29.435 | −1.513 | 1.00 | 0.47 | 1SG | 994 |
| ATOM | 994 | N | GLU | 118 | −2.205 | 30.898 | −1.723 | 1.00 | 0.70 | 1SG | 995 |
| ATOM | 995 | CA | GLU | 118 | −1.345 | 32.029 | −1.546 | 1.00 | 0.70 | 1SG | 996 |
| ATOM | 996 | CB | GLU | 118 | −1.883 | 33.322 | −2.185 | 1.00 | 0.70 | 1SG | 997 |
| ATOM | 997 | CG | GLU | 118 | −0.958 | 34.530 | −2.014 | 1.00 | 0.70 | 1SG | 998 |
| ATOM | 998 | CD | GLU | 118 | 0.182 | 34.405 | −3.015 | 1.00 | 0.70 | 1SG | 999 |
| ATOM | 999 | OE1 | GLU | 118 | 0.191 | 33.405 | −3.781 | 1.00 | 0.70 | 1SG | 1000 |
| ATOM | 1000 | OE2 | GLU | 118 | 1.058 | 35.310 | −3.029 | 1.00 | 0.70 | 1SG | 1001 |
| ATOM | 1001 | C | GLU | 118 | −1.260 | 32.258 | −0.075 | 1.00 | 0.70 | 1SG | 1002 |
| ATOM | 1002 | O | GLU | 118 | −2.278 | 32.324 | 0.614 | 1.00 | 0.70 | 1SG | 1003 |
| ATOM | 1003 | N | ALA | 119 | −0.029 | 32.366 | 0.453 | 1.00 | 0.64 | 1SG | 1004 |
| ATOM | 1004 | CA | ALA | 119 | 0.105 | 32.559 | 1.864 | 1.00 | 0.64 | 1SG | 1005 |
| ATOM | 1005 | CB | ALA | 119 | 0.342 | 31.252 | 2.638 | 1.00 | 0.64 | 1SG | 1006 |
| ATOM | 1006 | C | ALA | 119 | 1.300 | 33.419 | 2.094 | 1.00 | 0.64 | 1SG | 1007 |
| ATOM | 1007 | O | ALA | 119 | 1.618 | 34.301 | 1.297 | 1.00 | 0.64 | 1SG | 1008 |
| ATOM | 1008 | N | GLY | 120 | 1.980 | 33.177 | 3.230 | 1.00 | 0.62 | 1SG | 1009 |
| ATOM | 1009 | CA | GLY | 120 | 3.147 | 33.924 | 3.577 | 1.00 | 0.62 | 1SG | 1010 |
| ATOM | 1010 | C | GLY | 120 | 4.329 | 33.129 | 3.133 | 1.00 | 0.62 | 1SG | 1011 |
| ATOM | 1011 | O | GLY | 120 | 4.304 | 32.485 | 2.086 | 1.00 | 0.62 | 1SG | 1012 |
| ATOM | 1012 | N | PHE | 121 | 5.414 | 33.177 | 3.928 | 1.00 | 0.67 | 1SG | 1013 |
| ATOM | 1013 | CA | PHE | 121 | 6.625 | 32.493 | 3.588 | 1.00 | 0.67 | 1SG | 1014 |
| ATOM | 1014 | CB | PHE | 121 | 7.808 | 32.888 | 4.490 | 1.00 | 0.67 | 1SG | 1015 |
| ATOM | 1015 | CG | PHE | 121 | 8.086 | 34.329 | 4.238 | 1.00 | 0.67 | 1SG | 1016 |
| ATOM | 1016 | CD1 | PHE | 121 | 7.341 | 35.301 | 4.864 | 1.00 | 0.67 | 1SG | 1017 |
| ATOM | 1017 | CD2 | PHE | 121 | 9.092 | 34.707 | 3.380 | 1.00 | 0.67 | 1SG | 1018 |
| ATOM | 1018 | CE1 | PHE | 121 | 7.594 | 36.633 | 4.635 | 1.00 | 0.67 | 1SG | 1019 |
| ATOM | 1019 | CE2 | PHE | 121 | 9.350 | 36.037 | 3.147 | 1.00 | 0.67 | 1SG | 1020 |
| ATOM | 1020 | CZ | PHE | 121 | 8.600 | 37.002 | 3.774 | 1.00 | 0.67 | 1SG | 1021 |
| ATOM | 1021 | C | PHE | 121 | 6.442 | 31.005 | 3.682 | 1.00 | 0.67 | 1SG | 1022 |
| ATOM | 1022 | O | PHE | 121 | 6.871 | 30.287 | 2.780 | 1.00 | 0.67 | 1SG | 1023 |
| ATOM | 1023 | N | PRO | 122 | 5.827 | 30.493 | 4.718 | 1.00 | 0.49 | 1SG | 1024 |
| ATOM | 1024 | CA | PRO | 122 | 5.730 | 29.060 | 4.818 | 1.00 | 0.49 | 1SG | 1025 |
| ATOM | 1025 | CD | PRO | 122 | 5.903 | 31.114 | 6.029 | 1.00 | 0.49 | 1SG | 1026 |
| ATOM | 1026 | CB | PRO | 122 | 5.555 | 28.738 | 6.302 | 1.00 | 0.49 | 1SG | 1027 |
| ATOM | 1027 | CG | PRO | 122 | 5.247 | 30.091 | 6.967 | 1.00 | 0.49 | 1SG | 1028 |
| ATOM | 1028 | C | PRO | 122 | 4.680 | 28.464 | 3.937 | 1.00 | 0.49 | 1SG | 1029 |
| ATOM | 1029 | O | PRO | 122 | 3.497 | 28.567 | 4.260 | 1.00 | 0.49 | 1SG | 1030 |
| ATOM | 1030 | N | ILE | 123 | 5.101 | 27.817 | 2.834 | 1.00 | 0.27 | 1SG | 1031 |
| ATOM | 1031 | CA | ILE | 123 | 4.203 | 27.190 | 1.910 | 1.00 | 0.27 | 1SG | 1032 |
| ATOM | 1032 | CB | ILE | 123 | 4.881 | 26.851 | 0.614 | 1.00 | 0.27 | 1SG | 1033 |
| ATOM | 1033 | CG2 | ILE | 123 | 3.862 | 26.123 | −0.279 | 1.00 | 0.27 | 1SG | 1034 |
| ATOM | 1034 | CG1 | ILE | 123 | 5.473 | 28.116 | −0.031 | 1.00 | 0.27 | 1SG | 1035 |
| ATOM | 1035 | CD1 | ILE | 123 | 6.452 | 27.820 | −1.167 | 1.00 | 0.27 | 1SG | 1036 |
| ATOM | 1036 | C | ILE | 123 | 3.724 | 25.899 | 2.496 | 1.00 | 0.27 | 1SG | 1037 |
| ATOM | 1037 | O | ILE | 123 | 2.540 | 25.569 | 2.431 | 1.00 | 0.27 | 1SG | 1038 |
| ATOM | 1038 | N | ALA | 124 | 4.657 | 25.149 | 3.114 | 1.00 | 0.15 | 1SG | 1039 |
| ATOM | 1039 | CA | ALA | 124 | 4.393 | 23.825 | 3.595 | 1.00 | 0.15 | 1SG | 1040 |
| ATOM | 1040 | CB | ALA | 124 | 5.629 | 23.163 | 4.227 | 1.00 | 0.15 | 1SG | 1041 |
| ATOM | 1041 | C | ALA | 124 | 3.326 | 23.843 | 4.637 | 1.00 | 0.15 | 1SG | 1042 |
| ATOM | 1042 | O | ALA | 124 | 2.424 | 23.007 | 4.617 | 1.00 | 0.15 | 1SG | 1043 |
| ATOM | 1043 | N | TYR | 125 | 3.381 | 24.811 | 5.568 | 1.00 | 0.18 | 1SG | 1044 |
| ATOM | 1044 | CA | TYR | 125 | 2.432 | 24.812 | 6.644 | 1.00 | 0.18 | 1SG | 1045 |
| ATOM | 1045 | CB | TYR | 125 | 2.636 | 25.976 | 7.630 | 1.00 | 0.18 | 1SG | 1046 |
| ATOM | 1046 | CG | TYR | 125 | 3.925 | 25.765 | 8.351 | 1.00 | 0.18 | 1SG | 1047 |
| ATOM | 1047 | CD1 | TYR | 125 | 5.116 | 26.131 | 7.772 | 1.00 | 0.18 | 1SG | 1048 |
| ATOM | 1048 | CD2 | TYR | 125 | 3.944 | 25.207 | 9.609 | 1.00 | 0.18 | 1SG | 1049 |
| ATOM | 1049 | CE1 | TYR | 125 | 6.308 | 25.946 | 8.429 | 1.00 | 0.18 | 1SG | 1050 |
| ATOM | 1050 | CE2 | TYR | 125 | 5.133 | 25.017 | 10.275 | 1.00 | 0.18 | 1SG | 1051 |
| ATOM | 1051 | CZ | TYR | 125 | 6.317 | 25.388 | 9.684 | 1.00 | 0.18 | 1SG | 1052 |
| ATOM | 1052 | OH | TYR | 125 | 7.539 | 25.195 | 10.363 | 1.00 | 0.18 | 1SG | 1053 |
| ATOM | 1053 | C | TYR | 125 | 1.044 | 24.929 | 6.096 | 1.00 | 0.18 | 1SG | 1054 |
| ATOM | 1054 | O | TYR | 125 | 0.121 | 24.270 | 6.572 | 1.00 | 0.18 | 1SG | 1055 |
| ATOM | 1055 | N | SER | 126 | 0.840 | 25.790 | 5.089 | 1.00 | 0.37 | 1SG | 1056 |
| ATOM | 1056 | CA | SER | 126 | −0.483 | 25.928 | 4.553 | 1.00 | 0.37 | 1SG | 1057 |
| ATOM | 1057 | CB | SER | 126 | −0.585 | 27.058 | 3.512 | 1.00 | 0.37 | 1SG | 1058 |
| ATOM | 1058 | OG | SER | 126 | 0.247 | 26.776 | 2.396 | 1.00 | 0.37 | 1SG | 1059 |
| ATOM | 1059 | C | SER | 126 | −0.874 | 24.646 | 3.885 | 1.00 | 0.37 | 1SG | 1060 |
| ATOM | 1060 | O | SER | 126 | −1.990 | 24.156 | 4.050 | 1.00 | 0.37 | 1SG | 1061 |
| ATOM | 1061 | N | ILE | 127 | 0.050 | 24.047 | 3.117 | 1.00 | 0.52 | 1SG | 1062 |
| ATOM | 1062 | CA | ILE | 127 | −0.312 | 22.856 | 2.405 | 1.00 | 0.52 | 1SG | 1063 |
| ATOM | 1063 | CB | ILE | 127 | 0.808 | 22.318 | 1.558 | 1.00 | 0.52 | 1SG | 1064 |
| ATOM | 1064 | CG2 | ILE | 127 | 0.340 | 20.981 | 0.959 | 1.00 | 0.52 | 1SG | 1065 |
| ATOM | 1065 | CG1 | ILE | 127 | 1.235 | 23.346 | 0.495 | 1.00 | 0.52 | 1SG | 1066 |
| ATOM | 1066 | CD1 | ILE | 127 | 0.127 | 23.707 | −0.494 | 1.00 | 0.52 | 1SG | 1067 |
| ATOM | 1067 | C | ILE | 127 | −0.663 | 21.794 | 3.394 | 1.00 | 0.52 | 1SG | 1068 |
| ATOM | 1068 | O | ILE | 127 | −1.669 | 21.099 | 3.241 | 1.00 | 0.52 | 1SG | 1069 |
| ATOM | 1069 | N | VAL | 128 | 0.146 | 21.670 | 4.464 | 1.00 | 0.69 | 1SG | 1070 |
| ATOM | 1070 | CA | VAL | 128 | −0.087 | 20.607 | 5.391 | 1.00 | 0.69 | 1SG | 1071 |
| ATOM | 1071 | CB | VAL | 128 | 0.923 | 20.538 | 6.507 | 1.00 | 0.69 | 1SG | 1072 |
| ATOM | 1072 | CG1 | VAL | 128 | 0.765 | 21.756 | 7.424 | 1.00 | 0.69 | 1SG | 1073 |
| ATOM | 1073 | CG2 | VAL | 128 | 0.750 | 19.197 | 7.238 | 1.00 | 0.69 | 1SG | 1074 |
| ATOM | 1074 | C | VAL | 128 | −1.449 | 20.778 | 5.952 | 1.00 | 0.69 | 1SG | 1075 |
| ATOM | 1075 | O | VAL | 128 | −2.205 | 19.812 | 5.991 | 1.00 | 0.69 | 1SG | 1076 |
| ATOM | 1076 | N | VAL | 129 | −1.839 | 22.020 | 6.316 | 1.00 | 0.89 | 1SG | 1077 |
| ATOM | 1077 | CA | VAL | 129 | −3.145 | 22.236 | 6.876 | 1.00 | 0.89 | 1SG | 1078 |
| ATOM | 1078 | CB | VAL | 129 | −3.381 | 23.638 | 7.349 | 1.00 | 0.89 | 1SG | 1079 |
| ATOM | 1079 | CG1 | VAL | 129 | −2.178 | 24.053 | 8.214 | 1.00 | 0.89 | 1SG | 1080 |
| ATOM | 1080 | CG2 | VAL | 129 | −3.688 | 24.544 | 6.151 | 1.00 | 0.89 | 1SG | 1081 |
| ATOM | 1081 | C | VAL | 129 | −4.142 | 21.959 | 5.790 | 1.00 | 0.89 | 1SG | 1082 |
| ATOM | 1082 | O | VAL | 129 | −3.795 | 21.428 | 4.739 | 1.00 | 0.89 | 1SG | 1083 |
| ATOM | 1083 | N | HIS | 130 | −5.434 | 22.280 | 6.018 | 1.00 | 1.04 | 1SG | 1084 |
| ATOM | 1084 | CA | HIS | 130 | −6.499 | 22.020 | 5.073 | 1.00 | 1.04 | 1SG | 1085 |
| ATOM | 1085 | ND1 | HIS | 130 | −8.140 | 24.847 | 4.450 | 1.00 | 1.04 | 1SG | 1086 |
| ATOM | 1086 | CG | HIS | 130 | −6.849 | 24.399 | 4.282 | 1.00 | 1.04 | 1SG | 1087 |
| ATOM | 1087 | CB | HIS | 130 | −6.496 | 22.996 | 3.884 | 1.00 | 1.04 | 1SG | 1088 |
| ATOM | 1088 | NE2 | HIS | 130 | −6.806 | 26.568 | 4.901 | 1.00 | 1.04 | 1SG | 1089 |
| ATOM | 1089 | CD2 | HIS | 130 | −6.047 | 25.464 | 4.560 | 1.00 | 1.04 | 1SG | 1090 |
| ATOM | 1090 | CE1 | HIS | 130 | −8.055 | 26.149 | 4.819 | 1.00 | 1.04 | 1SG | 1091 |
| ATOM | 1091 | C | HIS | 130 | −6.376 | 20.617 | 4.550 | 1.00 | 1.04 | 1SG | 1092 |
| ATOM | 1092 | O | HIS | 130 | −6.790 | 20.297 | 3.438 | 1.00 | 1.04 | 1SG | 1093 |
| ATOM | 1093 | N | HIS | 131 | −5.799 | 19.755 | 5.395 | 1.00 | 1.19 | 1SG | 1094 |
| ATOM | 1094 | CA | HIS | 131 | −5.388 | 18.409 | 5.179 | 1.00 | 1.19 | 1SG | 1095 |
| ATOM | 1095 | ND1 | HIS | 131 | −4.557 | 15.943 | 2.875 | 1.00 | 1.19 | 1SG | 1096 |
| ATOM | 1096 | CG | HIS | 131 | −3.964 | 16.904 | 3.663 | 1.00 | 1.19 | 1SG | 1097 |
| ATOM | 1097 | CB | HIS | 131 | −4.520 | 18.276 | 3.912 | 1.00 | 1.19 | 1SG | 1098 |
| ATOM | 1098 | NE2 | HIS | 131 | −2.661 | 15.060 | 3.629 | 1.00 | 1.19 | 1SG | 1099 |
| ATOM | 1099 | CD2 | HIS | 131 | −2.807 | 16.346 | 4.117 | 1.00 | 1.19 | 1SG | 1100 |
| ATOM | 1100 | CE1 | HIS | 131 | −3.736 | 14.862 | 2.889 | 1.00 | 1.19 | 1SG | 1101 |
| ATOM | 1101 | C | HIS | 131 | −4.479 | 18.265 | 6.347 | 1.00 | 1.19 | 1SG | 1102 |
| ATOM | 1102 | O | HIS | 131 | −3.845 | 17.233 | 6.553 | 1.00 | 1.19 | 1SG | 1103 |
| ATOM | 1103 | N | LYS | 132 | −4.492 | 19.351 | 7.149 | 1.00 | 1.20 | 1SG | 1104 |
| ATOM | 1104 | CA | LYS | 132 | −3.679 | 19.708 | 8.280 | 1.00 | 1.20 | 1SG | 1105 |
| ATOM | 1105 | CB | LYS | 132 | −4.387 | 20.783 | 9.113 | 1.00 | 1.20 | 1SG | 1106 |
| ATOM | 1106 | CG | LYS | 132 | −3.540 | 21.784 | 9.868 | 1.00 | 1.20 | 1SG | 1107 |
| ATOM | 1107 | CD | LYS | 132 | −4.443 | 22.770 | 10.616 | 1.00 | 1.20 | 1SG | 1108 |
| ATOM | 1108 | CE | LYS | 132 | −4.320 | 24.227 | 10.220 | 1.00 | 1.20 | 1SG | 1109 |
| ATOM | 1109 | NZ | LYS | 132 | −3.172 | 24.808 | 10.934 | 1.00 | 1.20 | 1SG | 1110 |
| ATOM | 1110 | C | LYS | 132 | −3.382 | 18.506 | 9.104 | 1.00 | 1.20 | 1SG | 1111 |
| ATOM | 1111 | O | LYS | 132 | −4.109 | 17.520 | 9.054 | 1.00 | 1.20 | 1SG | 1112 |
| ATOM | 1112 | N | ILE | 133 | −2.287 | 18.563 | 9.900 | 1.00 | 1.06 | 1SG | 1113 |
| ATOM | 1113 | CA | ILE | 133 | −1.887 | 17.422 | 10.675 | 1.00 | 1.06 | 1SG | 1114 |
| ATOM | 1114 | CB | ILE | 133 | −0.442 | 17.490 | 11.096 | 1.00 | 1.06 | 1SG | 1115 |
| ATOM | 1115 | CG2 | ILE | 133 | −0.051 | 16.190 | 11.826 | 1.00 | 1.06 | 1SG | 1116 |
| ATOM | 1116 | CG1 | ILE | 133 | 0.433 | 17.706 | 9.850 | 1.00 | 1.06 | 1SG | 1117 |
| ATOM | 1117 | CD1 | ILE | 133 | 1.893 | 18.027 | 10.177 | 1.00 | 1.06 | 1SG | 1118 |
| ATOM | 1118 | C | ILE | 133 | −2.760 | 17.361 | 11.892 | 1.00 | 1.06 | 1SG | 1119 |
| ATOM | 1119 | O | ILE | 133 | −2.312 | 17.146 | 13.017 | 1.00 | 1.06 | 1SG | 1120 |
| ATOM | 1120 | N | GLU | 134 | −4.063 | 17.572 | 11.668 | 1.00 | 1.20 | 1SG | 1121 |
| ATOM | 1121 | CA | GLU | 134 | −5.095 | 17.459 | 12.647 | 1.00 | 1.20 | 1SG | 1122 |
| ATOM | 1122 | CB | GLU | 134 | −4.843 | 18.237 | 13.942 | 1.00 | 1.20 | 1SG | 1123 |
| ATOM | 1123 | CG | GLU | 134 | −5.931 | 17.973 | 14.982 | 1.00 | 1.20 | 1SG | 1124 |
| ATOM | 1124 | CD | GLU | 134 | −5.513 | 18.655 | 16.268 | 1.00 | 1.20 | 1SG | 1125 |
| ATOM | 1125 | OE1 | GLU | 134 | −4.647 | 19.569 | 16.187 | 1.00 | 1.20 | 1SG | 1126 |
| ATOM | 1126 | OE2 | GLU | 134 | −6.048 | 18.276 | 17.340 | 1.00 | 1.20 | 1SG | 1127 |
| ATOM | 1127 | C | GLU | 134 | −6.258 | 18.090 | 11.984 | 1.00 | 1.20 | 1SG | 1128 |
| ATOM | 1128 | O | GLU | 134 | −7.385 | 17.601 | 12.049 | 1.00 | 1.20 | 1SG | 1129 |
| ATOM | 1129 | N | MET | 135 | −5.984 | 19.205 | 11.286 | 1.00 | 1.28 | 1SG | 1130 |
| ATOM | 1130 | CA | MET | 135 | −7.042 | 19.802 | 10.550 | 1.00 | 1.28 | 1SG | 1131 |
| ATOM | 1131 | CB | MET | 135 | −7.001 | 21.333 | 10.484 | 1.00 | 1.28 | 1SG | 1132 |
| ATOM | 1132 | CG | MET | 135 | −8.257 | 21.936 | 9.859 | 1.00 | 1.28 | 1SG | 1133 |
| ATOM | 1133 | SD | MET | 135 | −9.764 | 21.695 | 10.845 | 1.00 | 1.28 | 1SG | 1134 |
| ATOM | 1134 | CE | MET | 135 | −9.993 | 19.988 | 10.278 | 1.00 | 1.28 | 1SG | 1135 |
| ATOM | 1135 | C | MET | 135 | −6.888 | 19.270 | 9.173 | 1.00 | 1.28 | 1SG | 1136 |
| ATOM | 1136 | O | MET | 135 | −6.826 | 19.998 | 8.183 | 1.00 | 1.28 | 1SG | 1137 |
| ATOM | 1137 | N | LEU | 136 | −6.902 | 17.930 | 9.090 | 1.00 | 0.96 | 1SG | 1138 |
| ATOM | 1138 | CA | LEU | 136 | −6.792 | 17.117 | 7.925 | 1.00 | 0.96 | 1SG | 1139 |
| ATOM | 1139 | CB | LEU | 136 | −6.800 | 15.592 | 8.191 | 1.00 | 0.96 | 1SG | 1140 |
| ATOM | 1140 | CG | LEU | 136 | −5.597 | 14.946 | 8.912 | 1.00 | 0.96 | 1SG | 1141 |
| ATOM | 1141 | CD2 | LEU | 136 | −5.423 | 15.483 | 10.342 | 1.00 | 0.96 | 1SG | 1142 |
| ATOM | 1142 | CD1 | LEU | 136 | −4.326 | 14.988 | 8.050 | 1.00 | 0.96 | 1SG | 1143 |
| ATOM | 1143 | C | LEU | 136 | −8.022 | 17.349 | 7.117 | 1.00 | 0.96 | 1SG | 1144 |
| ATOM | 1144 | O | LEU | 136 | −8.169 | 16.727 | 6.081 | 1.00 | 0.96 | 1SG | 1145 |
| ATOM | 1145 | N | ASP | 137 | −8.910 | 18.269 | 7.551 | 1.00 | 0.66 | 1SG | 1146 |
| ATOM | 1146 | CA | ASP | 137 | −10.289 | 18.473 | 7.222 | 1.00 | 0.66 | 1SG | 1147 |
| ATOM | 1147 | CB | ASP | 137 | −10.730 | 19.940 | 7.386 | 1.00 | 0.66 | 1SG | 1148 |
| ATOM | 1148 | CG | ASP | 137 | −9.976 | 20.778 | 6.354 | 1.00 | 0.66 | 1SG | 1149 |
| ATOM | 1149 | OD1 | ASP | 137 | −8.807 | 20.432 | 6.039 | 1.00 | 0.66 | 1SG | 1150 |
| ATOM | 1150 | OD2 | ASP | 137 | −10.557 | 21.788 | 5.868 | 1.00 | 0.66 | 1SG | 1151 |
| ATOM | 1151 | C | ASP | 137 | −10.644 | 18.085 | 5.820 | 1.00 | 0.66 | 1SG | 1152 |
| ATOM | 1152 | O | ASP | 137 | −11.740 | 17.560 | 5.629 | 1.00 | 0.66 | 1SG | 1153 |
| ATOM | 1153 | N | ARG | 138 | −9.811 | 18.339 | 4.791 | 1.00 | 0.54 | 1SG | 1154 |
| ATOM | 1154 | CA | ARG | 138 | −10.267 | 17.894 | 3.495 | 1.00 | 0.54 | 1SG | 1155 |
| ATOM | 1155 | CB | ARG | 138 | −9.298 | 18.222 | 2.347 | 1.00 | 0.54 | 1SG | 1156 |
| ATOM | 1156 | CG | ARG | 138 | −9.108 | 19.717 | 2.075 | 1.00 | 0.54 | 1SG | 1157 |
| ATOM | 1157 | CD | ARG | 138 | −8.189 | 19.991 | 0.879 | 1.00 | 0.54 | 1SG | 1158 |
| ATOM | 1158 | NE | ARG | 138 | −7.876 | 21.449 | 0.857 | 1.00 | 0.54 | 1SG | 1159 |
| ATOM | 1159 | CZ | ARG | 138 | −8.738 | 22.338 | 0.288 | 1.00 | 0.54 | 1SG | 1160 |
| ATOM | 1160 | NH1 | ARG | 138 | −9.949 | 21.916 | −0.183 | 1.00 | 0.54 | 1SG | 1161 |
| ATOM | 1161 | NH2 | ARG | 138 | −8.386 | 23.656 | 0.197 | 1.00 | 0.54 | 1SG | 1162 |
| ATOM | 1162 | C | ARG | 138 | −10.439 | 16.395 | 3.536 | 1.00 | 0.54 | 1SG | 1163 |
| ATOM | 1163 | O | ARG | 138 | −11.459 | 15.853 | 3.106 | 1.00 | 0.54 | 1SG | 1164 |
| ATOM | 1164 | N | LEU | 139 | −9.431 | 15.702 | 4.096 | 1.00 | 0.46 | 1SG | 1165 |
| ATOM | 1165 | CA | LEU | 139 | −9.362 | 14.282 | 4.320 | 1.00 | 0.46 | 1SG | 1166 |
| ATOM | 1166 | CB | LEU | 139 | −7.985 | 13.847 | 4.857 | 1.00 | 0.46 | 1SG | 1167 |
| ATOM | 1167 | CG | LEU | 139 | −7.697 | 12.335 | 4.820 | 1.00 | 0.46 | 1SG | 1168 |
| ATOM | 1168 | CD2 | LEU | 139 | −8.738 | 11.471 | 5.538 | 1.00 | 0.46 | 1SG | 1169 |
| ATOM | 1169 | CD1 | LEU | 139 | −6.267 | 12.058 | 5.303 | 1.00 | 0.46 | 1SG | 1170 |
| ATOM | 1170 | C | LEU | 139 | −10.409 | 13.884 | 5.325 | 1.00 | 0.46 | 1SG | 1171 |
| ATOM | 1171 | O | LEU | 139 | −11.077 | 12.865 | 5.155 | 1.00 | 0.46 | 1SG | 1172 |
| ATOM | 1172 | N | LEU | 140 | −10.598 | 14.671 | 6.406 | 1.00 | 0.61 | 1SG | 1173 |
| ATOM | 1173 | CA | LEU | 140 | −11.583 | 14.234 | 7.366 | 1.00 | 0.61 | 1SG | 1174 |
| ATOM | 1174 | CB | LEU | 140 | −11.821 | 15.140 | 8.586 | 1.00 | 0.61 | 1SG | 1175 |
| ATOM | 1175 | CG | LEU | 140 | −10.724 | 15.147 | 9.659 | 1.00 | 0.61 | 1SG | 1176 |
| ATOM | 1176 | CD2 | LEU | 140 | −11.281 | 15.587 | 11.021 | 1.00 | 0.61 | 1SG | 1177 |
| ATOM | 1177 | CD1 | LEU | 140 | −9.518 | 15.970 | 9.234 | 1.00 | 0.61 | 1SG | 1178 |
| ATOM | 1178 | C | LEU | 140 | −12.912 | 14.207 | 6.698 | 1.00 | 0.61 | 1SG | 1179 |
| ATOM | 1179 | O | LEU | 140 | −13.741 | 13.334 | 6.958 | 1.00 | 0.61 | 1SG | 1180 |
| ATOM | 1180 | N | ARG | 141 | −13.158 | 15.196 | 5.826 | 1.00 | 0.69 | 1SG | 1181 |
| ATOM | 1181 | CA | ARG | 141 | −14.416 | 15.238 | 5.149 | 1.00 | 0.69 | 1SG | 1182 |
| ATOM | 1182 | CB | ARG | 141 | −14.536 | 16.402 | 4.150 | 1.00 | 0.69 | 1SG | 1183 |
| ATOM | 1183 | CG | ARG | 141 | −14.533 | 17.778 | 4.820 | 1.00 | 0.69 | 1SG | 1184 |
| ATOM | 1184 | CD | ARG | 141 | −14.827 | 18.927 | 3.850 | 1.00 | 0.69 | 1SG | 1185 |
| ATOM | 1185 | NE | ARG | 141 | −16.252 | 18.814 | 3.425 | 1.00 | 0.69 | 1SG | 1186 |
| ATOM | 1186 | CZ | ARG | 141 | −16.791 | 19.726 | 2.562 | 1.00 | 0.69 | 1SG | 1187 |
| ATOM | 1187 | NH1 | ARG | 141 | −16.040 | 20.768 | 2.097 | 1.00 | 0.69 | 1SG | 1188 |
| ATOM | 1188 | NH2 | ARG | 141 | −18.086 | 19.589 | 2.155 | 1.00 | 0.69 | 1SG | 1189 |
| ATOM | 1189 | C | ARG | 141 | −14.534 | 13.977 | 4.364 | 1.00 | 0.69 | 1SG | 1190 |
| ATOM | 1190 | O | ARG | 141 | −15.602 | 13.368 | 4.314 | 1.00 | 0.69 | 1SG | 1191 |
| ATOM | 1191 | N | ALA | 142 | −13.413 | 13.543 | 3.759 | 1.00 | 0.48 | 1SG | 1192 |
| ATOM | 1192 | CA | ALA | 142 | −13.400 | 12.364 | 2.948 | 1.00 | 0.48 | 1SG | 1193 |
| ATOM | 1193 | CB | ALA | 142 | −11.999 | 12.041 | 2.392 | 1.00 | 0.48 | 1SG | 1194 |
| ATOM | 1194 | C | ALA | 142 | −13.820 | 11.204 | 3.784 | 1.00 | 0.48 | 1SG | 1195 |
| ATOM | 1195 | O | ALA | 142 | −14.623 | 10.385 | 3.337 | 1.00 | 0.48 | 1SG | 1196 |
| ATOM | 1196 | N | ILE | 143 | −13.324 | 11.106 | 5.032 | 1.00 | 0.46 | 1SG | 1197 |
| ATOM | 1197 | CA | ILE | 143 | −13.677 | 9.955 | 5.801 | 1.00 | 0.46 | 1SG | 1198 |
| ATOM | 1198 | CB | ILE | 143 | −13.013 | 9.821 | 7.133 | 1.00 | 0.46 | 1SG | 1199 |
| ATOM | 1199 | CG2 | ILE | 143 | −13.630 | 10.847 | 8.102 | 1.00 | 0.46 | 1SG | 1200 |
| ATOM | 1200 | CG1 | ILE | 143 | −13.202 | 8.359 | 7.577 | 1.00 | 0.46 | 1SG | 1201 |
| ATOM | 1201 | CD1 | ILE | 143 | −12.144 | 7.818 | 8.535 | 1.00 | 0.46 | 1SG | 1202 |
| ATOM | 1202 | C | ILE | 143 | −15.142 | 9.974 | 6.006 | 1.00 | 0.46 | 1SG | 1203 |
| ATOM | 1203 | O | ILE | 143 | −15.734 | 8.926 | 6.230 | 1.00 | 0.46 | 1SG | 1204 |
| ATOM | 1204 | N | TYR | 144 | −15.763 | 11.166 | 5.969 | 1.00 | 0.57 | 1SG | 1205 |
| ATOM | 1205 | CA | TYR | 144 | −17.190 | 11.294 | 6.070 | 1.00 | 0.57 | 1SG | 1206 |
| ATOM | 1206 | CB | TYR | 144 | −17.699 | 12.740 | 6.111 | 1.00 | 0.57 | 1SG | 1207 |
| ATOM | 1207 | CG | TYR | 144 | −17.527 | 13.196 | 7.506 | 1.00 | 0.57 | 1SG | 1208 |
| ATOM | 1208 | CD1 | TYR | 144 | −16.333 | 13.731 | 7.927 | 1.00 | 0.57 | 1SG | 1209 |
| ATOM | 1209 | CD2 | TYR | 144 | −18.568 | 13.071 | 8.391 | 1.00 | 0.57 | 1SG | 1210 |
| ATOM | 1210 | CE1 | TYR | 144 | −16.176 | 14.141 | 9.227 | 1.00 | 0.57 | 1SG | 1211 |
| ATOM | 1211 | CE2 | TYR | 144 | −18.410 | 13.483 | 9.692 | 1.00 | 0.57 | 1SG | 1212 |
| ATOM | 1212 | CZ | TYR | 144 | −17.220 | 14.018 | 10.114 | 1.00 | 0.57 | 1SG | 1213 |
| ATOM | 1213 | OH | TYR | 144 | −17.069 | 14.437 | 11.453 | 1.00 | 0.57 | 1SG | 1214 |
| ATOM | 1214 | C | TYR | 144 | −17.858 | 10.666 | 4.895 | 1.00 | 0.57 | 1SG | 1215 |
| ATOM | 1215 | O | TYR | 144 | −18.966 | 10.153 | 5.045 | 1.00 | 0.57 | 1SG | 1216 |
| ATOM | 1216 | N | MET | 145 | −17.202 | 10.679 | 3.710 | 1.00 | 0.55 | 1SG | 1217 |
| ATOM | 1217 | CA | MET | 145 | −17.802 | 10.136 | 2.522 | 1.00 | 0.55 | 1SG | 1218 |
| ATOM | 1218 | CB | MET | 145 | −16.849 | 10.139 | 1.303 | 1.00 | 0.55 | 1SG | 1219 |
| ATOM | 1219 | CG | MET | 145 | −16.623 | 11.560 | 0.746 | 1.00 | 0.55 | 1SG | 1220 |
| ATOM | 1220 | SD | MET | 145 | −15.504 | 11.688 | −0.689 | 1.00 | 0.55 | 1SG | 1221 |
| ATOM | 1221 | CE | MET | 145 | −15.835 | 13.430 | −1.088 | 1.00 | 0.55 | 1SG | 1222 |
| ATOM | 1222 | C | MET | 145 | −18.205 | 8.737 | 2.856 | 1.00 | 0.55 | 1SG | 1223 |
| ATOM | 1223 | O | MET | 145 | −19.301 | 8.296 | 2.501 | 1.00 | 0.55 | 1SG | 1224 |
| ATOM | 1224 | N | PRO | 146 | −17.378 | 8.007 | 3.539 | 1.00 | 0.70 | 1SG | 1225 |
| ATOM | 1225 | CA | PRO | 146 | −17.871 | 6.771 | 4.062 | 1.00 | 0.70 | 1SG | 1226 |
| ATOM | 1226 | CD | PRO | 146 | −15.971 | 7.907 | 3.186 | 1.00 | 0.70 | 1SG | 1227 |
| ATOM | 1227 | CB | PRO | 146 | −16.648 | 5.898 | 4.358 | 1.00 | 0.70 | 1SG | 1228 |
| ATOM | 1228 | CG | PRO | 146 | −15.433 | 6.826 | 4.141 | 1.00 | 0.70 | 1SG | 1229 |
| ATOM | 1229 | C | PRO | 146 | −18.539 | 7.247 | 5.299 | 1.00 | 0.70 | 1SG | 1230 |
| ATOM | 1230 | O | PRO | 146 | −17.815 | 7.534 | 6.239 | 1.00 | 0.70 | 1SG | 1231 |
| ATOM | 1231 | N | GLN | 147 | −19.874 | 7.326 | 5.380 | 1.00 | 0.87 | 1SG | 1232 |
| ATOM | 1232 | CA | GLN | 147 | −20.424 | 7.905 | 6.572 | 1.00 | 0.87 | 1SG | 1233 |
| ATOM | 1233 | CB | GLN | 147 | −21.962 | 8.039 | 6.513 | 1.00 | 0.87 | 1SG | 1234 |
| ATOM | 1234 | CG | GLN | 147 | −22.765 | 6.741 | 6.309 | 1.00 | 0.87 | 1SG | 1235 |
| ATOM | 1235 | CD | GLN | 147 | −22.882 | 6.486 | 4.805 | 1.00 | 0.87 | 1SG | 1236 |
| ATOM | 1236 | OE1 | GLN | 147 | −23.412 | 7.305 | 4.051 | 1.00 | 0.87 | 1SG | 1237 |
| ATOM | 1237 | NE2 | GLN | 147 | −22.367 | 5.317 | 4.341 | 1.00 | 0.87 | 1SG | 1238 |
| ATOM | 1238 | C | GLN | 147 | −20.066 | 7.054 | 7.746 | 1.00 | 0.87 | 1SG | 1239 |
| ATOM | 1239 | O | GLN | 147 | −20.707 | 6.040 | 7.997 | 1.00 | 0.87 | 1SG | 1240 |
| ATOM | 1240 | N | ASN | 148 | −19.019 | 7.412 | 8.515 | 1.00 | 0.73 | 1SG | 1241 |
| ATOM | 1241 | CA | ASN | 148 | −18.657 | 6.585 | 9.618 | 1.00 | 0.73 | 1SG | 1242 |
| ATOM | 1242 | CB | ASN | 148 | −17.585 | 5.529 | 9.297 | 1.00 | 0.73 | 1SG | 1243 |
| ATOM | 1243 | CG | ASN | 148 | −17.777 | 4.444 | 10.362 | 1.00 | 0.73 | 1SG | 1244 |
| ATOM | 1244 | OD1 | ASN | 148 | −16.842 | 3.769 | 10.781 | 1.00 | 0.73 | 1SG | 1245 |
| ATOM | 1245 | ND2 | ASN | 148 | −19.050 | 4.263 | 10.796 | 1.00 | 0.73 | 1SG | 1246 |
| ATOM | 1246 | C | ASN | 148 | −18.139 | 7.472 | 10.698 | 1.00 | 0.73 | 1SG | 1247 |
| ATOM | 1247 | O | ASN | 148 | −17.276 | 8.315 | 10.477 | 1.00 | 0.73 | 1SG | 1248 |
| ATOM | 1248 | N | PHE | 149 | −18.666 | 7.275 | 11.927 | 1.00 | 0.57 | 1SG | 1249 |
| ATOM | 1249 | CA | PHE | 149 | −18.370 | 8.035 | 13.104 | 1.00 | 0.57 | 1SG | 1250 |
| ATOM | 1250 | CB | PHE | 149 | −19.385 | 7.802 | 14.249 | 1.00 | 0.57 | 1SG | 1251 |
| ATOM | 1251 | CG | PHE | 149 | −20.545 | 8.743 | 14.165 | 1.00 | 0.57 | 1SG | 1252 |
| ATOM | 1252 | CD1 | PHE | 149 | −21.627 | 8.586 | 13.328 | 1.00 | 0.57 | 1SG | 1253 |
| ATOM | 1253 | CD2 | PHE | 149 | −20.519 | 9.825 | 15.010 | 1.00 | 0.57 | 1SG | 1254 |
| ATOM | 1254 | CE1 | PHE | 149 | −22.645 | 9.517 | 13.346 | 1.00 | 0.57 | 1SG | 1255 |
| ATOM | 1255 | CE2 | PHE | 149 | −21.525 | 10.755 | 15.034 | 1.00 | 0.57 | 1SG | 1256 |
| ATOM | 1256 | CZ | PHE | 149 | −22.598 | 10.595 | 14.196 | 1.00 | 0.57 | 1SG | 1257 |
| ATOM | 1257 | C | PHE | 149 | −16.973 | 7.844 | 13.653 | 1.00 | 0.57 | 1SG | 1258 |
| ATOM | 1258 | O | PHE | 149 | −16.513 | 8.666 | 14.447 | 1.00 | 0.57 | 1SG | 1259 |
| ATOM | 1259 | N | TYR | 150 | −16.249 | 6.740 | 13.370 | 1.00 | 0.60 | 1SG | 1260 |
| ATOM | 1260 | CA | TYR | 150 | −14.970 | 6.700 | 14.029 | 1.00 | 0.60 | 1SG | 1261 |
| ATOM | 1261 | CB | TYR | 150 | −14.752 | 5.456 | 14.921 | 1.00 | 0.60 | 1SG | 1262 |
| ATOM | 1262 | CG | TYR | 150 | −15.870 | 5.363 | 15.893 | 1.00 | 0.60 | 1SG | 1263 |
| ATOM | 1263 | CD1 | TYR | 150 | −15.840 | 6.069 | 17.073 | 1.00 | 0.60 | 1SG | 1264 |
| ATOM | 1264 | CD2 | TYR | 150 | −16.956 | 4.572 | 15.601 | 1.00 | 0.60 | 1SG | 1265 |
| ATOM | 1265 | CE1 | TYR | 150 | −16.890 | 5.963 | 17.954 | 1.00 | 0.60 | 1SG | 1266 |
| ATOM | 1266 | CE2 | TYR | 150 | −18.004 | 4.463 | 16.478 | 1.00 | 0.60 | 1SG | 1267 |
| ATOM | 1267 | CZ | TYR | 150 | −17.967 | 5.163 | 17.658 | 1.00 | 0.60 | 1SG | 1268 |
| ATOM | 1268 | OH | TYR | 150 | −19.035 | 5.064 | 18.575 | 1.00 | 0.60 | 1SG | 1269 |
| ATOM | 1269 | C | TYR | 150 | −13.870 | 6.621 | 13.013 | 1.00 | 0.60 | 1SG | 1270 |
| ATOM | 1270 | O | TYR | 150 | −14.086 | 6.229 | 11.869 | 1.00 | 0.60 | 1SG | 1271 |
| ATOM | 1271 | N | CYS | 151 | −12.652 | 7.051 | 13.406 | 1.00 | 0.62 | 1SG | 1272 |
| ATOM | 1272 | CA | CYS | 151 | −11.523 | 6.867 | 12.552 | 1.00 | 0.62 | 1SG | 1273 |
| ATOM | 1273 | CB | CYS | 151 | −11.402 | 7.872 | 11.384 | 1.00 | 0.62 | 1SG | 1274 |
| ATOM | 1274 | SG | CYS | 151 | −10.383 | 9.316 | 11.800 | 1.00 | 0.62 | 1SG | 1275 |
| ATOM | 1275 | C | CYS | 151 | −10.306 | 7.061 | 13.394 | 1.00 | 0.62 | 1SG | 1276 |
| ATOM | 1276 | O | CYS | 151 | −10.311 | 7.801 | 14.378 | 1.00 | 0.62 | 1SG | 1277 |
| ATOM | 1277 | N | ILE | 152 | −9.211 | 6.384 | 13.031 | 1.00 | 0.50 | 1SG | 1278 |
| ATOM | 1278 | CA | ILE | 152 | −8.028 | 6.608 | 13.785 | 1.00 | 0.50 | 1SG | 1279 |
| ATOM | 1279 | CB | ILE | 152 | −7.450 | 5.339 | 14.326 | 1.00 | 0.50 | 1SG | 1280 |
| ATOM | 1280 | CG2 | ILE | 152 | −6.124 | 5.675 | 15.024 | 1.00 | 0.50 | 1SG | 1281 |
| ATOM | 1281 | CG1 | ILE | 152 | −8.492 | 4.661 | 15.236 | 1.00 | 0.50 | 1SG | 1282 |
| ATOM | 1282 | CD1 | ILE | 152 | −9.024 | 5.574 | 16.343 | 1.00 | 0.50 | 1SG | 1283 |
| ATOM | 1283 | C | ILE | 152 | −7.076 | 7.234 | 12.830 | 1.00 | 0.50 | 1SG | 1284 |
| ATOM | 1284 | O | ILE | 152 | −6.775 | 6.668 | 11.781 | 1.00 | 0.50 | 1SG | 1285 |
| ATOM | 1285 | N | HIS | 153 | −6.577 | 8.440 | 13.144 | 1.00 | 0.29 | 1SG | 1286 |
| ATOM | 1286 | CA | HIS | 153 | −5.711 | 8.978 | 12.144 | 1.00 | 0.29 | 1SG | 1287 |
| ATOM | 1287 | ND1 | HIS | 153 | −7.874 | 11.933 | 11.693 | 1.00 | 0.29 | 1SG | 1288 |
| ATOM | 1288 | CG | HIS | 153 | −7.071 | 10.915 | 11.233 | 1.00 | 0.29 | 1SG | 1289 |
| ATOM | 1289 | CB | HIS | 153 | −5.791 | 10.492 | 11.887 | 1.00 | 0.29 | 1SG | 1290 |
| ATOM | 1290 | NE2 | HIS | 153 | −8.848 | 11.119 | 9.864 | 1.00 | 0.29 | 1SG | 1291 |
| ATOM | 1291 | CD2 | HIS | 153 | −7.677 | 10.430 | 10.119 | 1.00 | 0.29 | 1SG | 1292 |
| ATOM | 1292 | CE1 | HIS | 153 | −8.922 | 12.010 | 10.832 | 1.00 | 0.29 | 1SG | 1293 |
| ATOM | 1293 | C | HIS | 153 | −4.314 | 8.659 | 12.521 | 1.00 | 0.29 | 1SG | 1294 |
| ATOM | 1294 | O | HIS | 153 | −3.949 | 8.656 | 13.696 | 1.00 | 0.29 | 1SG | 1295 |
| ATOM | 1295 | N | VAL | 154 | −3.532 | 8.308 | 11.484 | 1.00 | 0.25 | 1SG | 1296 |
| ATOM | 1296 | CA | VAL | 154 | −2.138 | 8.026 | 11.603 | 1.00 | 0.25 | 1SG | 1297 |
| ATOM | 1297 | CB | VAL | 154 | −1.808 | 6.580 | 11.380 | 1.00 | 0.25 | 1SG | 1298 |
| ATOM | 1298 | CG1 | VAL | 154 | −0.283 | 6.439 | 11.271 | 1.00 | 0.25 | 1SG | 1299 |
| ATOM | 1299 | CG2 | VAL | 154 | −2.454 | 5.757 | 12.511 | 1.00 | 0.25 | 1SG | 1300 |
| ATOM | 1300 | C | VAL | 154 | −1.471 | 8.811 | 10.521 | 1.00 | 0.25 | 1SG | 1301 |
| ATOM | 1301 | O | VAL | 154 | −1.958 | 8.890 | 9.398 | 1.00 | 0.25 | 1SG | 1302 |
| ATOM | 1302 | N | ASP | 155 | −0.329 | 9.440 | 10.830 | 1.00 | 0.31 | 1SG | 1303 |
| ATOM | 1303 | CA | ASP | 155 | 0.320 | 10.234 | 9.833 | 1.00 | 0.31 | 1SG | 1304 |
| ATOM | 1304 | CB | ASP | 155 | 0.956 | 11.537 | 10.364 | 1.00 | 0.31 | 1SG | 1305 |
| ATOM | 1305 | CG | ASP | 155 | 1.134 | 12.558 | 9.247 | 1.00 | 0.31 | 1SG | 1306 |
| ATOM | 1306 | OD1 | ASP | 155 | 1.979 | 12.343 | 8.339 | 1.00 | 0.31 | 1SG | 1307 |
| ATOM | 1307 | OD2 | ASP | 155 | 0.412 | 13.590 | 9.295 | 1.00 | 0.31 | 1SG | 1308 |
| ATOM | 1308 | C | ASP | 155 | 1.413 | 9.377 | 9.332 | 1.00 | 0.31 | 1SG | 1309 |
| ATOM | 1309 | O | ASP | 155 | 1.795 | 8.404 | 9.978 | 1.00 | 0.31 | 1SG | 1310 |
| ATOM | 1310 | N | ALA | 156 | 1.947 | 9.741 | 8.161 | 1.00 | 0.26 | 1SG | 1311 |
| ATOM | 1311 | CA | ALA | 156 | 2.988 | 9.001 | 7.523 | 1.00 | 0.26 | 1SG | 1312 |
| ATOM | 1312 | CB | ALA | 156 | 3.433 | 9.654 | 6.208 | 1.00 | 0.26 | 1SG | 1313 |
| ATOM | 1313 | C | ALA | 156 | 4.183 | 8.981 | 8.425 | 1.00 | 0.26 | 1SG | 1314 |
| ATOM | 1314 | O | ALA | 156 | 4.935 | 8.009 | 8.461 | 1.00 | 0.26 | 1SG | 1315 |
| ATOM | 1315 | N | LYS | 157 | 4.393 | 10.082 | 9.161 | 1.00 | 0.30 | 1SG | 1316 |
| ATOM | 1316 | CA | LYS | 157 | 5.524 | 10.268 | 10.019 | 1.00 | 0.30 | 1SG | 1317 |
| ATOM | 1317 | CB | LYS | 157 | 5.639 | 11.735 | 10.472 | 1.00 | 0.30 | 1SG | 1318 |
| ATOM | 1318 | CG | LYS | 157 | 5.850 | 12.667 | 9.274 | 1.00 | 0.30 | 1SG | 1319 |
| ATOM | 1319 | CD | LYS | 157 | 5.519 | 14.137 | 9.537 | 1.00 | 0.30 | 1SG | 1320 |
| ATOM | 1320 | CE | LYS | 157 | 5.446 | 14.969 | 8.254 | 1.00 | 0.30 | 1SG | 1321 |
| ATOM | 1321 | NZ | LYS | 157 | 4.902 | 16.313 | 8.549 | 1.00 | 0.30 | 1SG | 1322 |
| ATOM | 1322 | C | LYS | 157 | 5.456 | 9.366 | 11.216 | 1.00 | 0.30 | 1SG | 1323 |
| ATOM | 1323 | O | LYS | 157 | 6.483 | 9.039 | 11.806 | 1.00 | 0.30 | 1SG | 1324 |
| ATOM | 1324 | N | SER | 158 | 4.245 | 8.966 | 11.636 | 1.00 | 0.45 | 1SG | 1325 |
| ATOM | 1325 | CA | SER | 158 | 4.129 | 8.160 | 12.817 | 1.00 | 0.45 | 1SG | 1326 |
| ATOM | 1326 | CB | SER | 158 | 2.735 | 8.249 | 13.447 | 1.00 | 0.45 | 1SG | 1327 |
| ATOM | 1327 | OG | SER | 158 | 2.482 | 9.586 | 13.850 | 1.00 | 0.45 | 1SG | 1328 |
| ATOM | 1328 | C | SER | 158 | 4.395 | 6.721 | 12.501 | 1.00 | 0.45 | 1SG | 1329 |
| ATOM | 1329 | O | SER | 158 | 4.262 | 6.267 | 11.367 | 1.00 | 0.45 | 1SG | 1330 |
| ATOM | 1330 | N | GLU | 159 | 4.820 | 5.966 | 13.532 | 1.00 | 0.60 | 1SG | 1331 |
| ATOM | 1331 | CA | GLU | 159 | 5.052 | 4.559 | 13.404 | 1.00 | 0.60 | 1SG | 1332 |
| ATOM | 1332 | CB | GLU | 159 | 6.545 | 4.197 | 13.291 | 1.00 | 0.60 | 1SG | 1333 |
| ATOM | 1333 | CG | GLU | 159 | 6.805 | 2.706 | 13.069 | 1.00 | 0.60 | 1SG | 1334 |
| ATOM | 1334 | CD | GLU | 159 | 8.312 | 2.499 | 12.976 | 1.00 | 0.60 | 1SG | 1335 |
| ATOM | 1335 | OE1 | GLU | 159 | 9.000 | 3.398 | 12.426 | 1.00 | 0.60 | 1SG | 1336 |
| ATOM | 1336 | OE2 | GLU | 159 | 8.795 | 1.437 | 13.455 | 1.00 | 0.60 | 1SG | 1337 |
| ATOM | 1337 | C | GLU | 159 | 4.557 | 3.982 | 14.682 | 1.00 | 0.60 | 1SG | 1338 |
| ATOM | 1338 | O | GLU | 159 | 4.937 | 4.438 | 15.759 | 1.00 | 0.60 | 1SG | 1339 |
| ATOM | 1339 | N | LYS | 160 | 3.669 | 2.977 | 14.618 | 1.00 | 0.71 | 1SG | 1340 |
| ATOM | 1340 | CA | LYS | 160 | 3.259 | 2.455 | 15.882 | 1.00 | 0.71 | 1SG | 1341 |
| ATOM | 1341 | CB | LYS | 160 | 1.768 | 2.618 | 16.210 | 1.00 | 0.71 | 1SG | 1342 |
| ATOM | 1342 | CG | LYS | 160 | 1.512 | 2.520 | 17.716 | 1.00 | 0.71 | 1SG | 1343 |
| ATOM | 1343 | CD | LYS | 160 | 0.151 | 3.059 | 18.156 | 1.00 | 0.71 | 1SG | 1344 |
| ATOM | 1344 | CE | LYS | 160 | 0.045 | 3.299 | 19.666 | 1.00 | 0.71 | 1SG | 1345 |
| ATOM | 1345 | NZ | LYS | 160 | 0.152 | 2.020 | 20.404 | 1.00 | 0.71 | 1SG | 1346 |
| ATOM | 1346 | C | LYS | 160 | 3.625 | 1.009 | 15.927 | 1.00 | 0.71 | 1SG | 1347 |
| ATOM | 1347 | O | LYS | 160 | 3.894 | 0.393 | 14.897 | 1.00 | 0.71 | 1SG | 1348 |
| ATOM | 1348 | N | SER | 161 | 3.664 | 0.438 | 17.148 | 1.00 | 0.81 | 1SG | 1349 |
| ATOM | 1349 | CA | SER | 161 | 4.088 | −0.922 | 17.312 | 1.00 | 0.81 | 1SG | 1350 |
| ATOM | 1350 | CB | SER | 161 | 4.185 | −1.367 | 18.784 | 1.00 | 0.81 | 1SG | 1351 |
| ATOM | 1351 | OG | SER | 161 | 5.181 | −0.612 | 19.462 | 1.00 | 0.81 | 1SG | 1352 |
| ATOM | 1352 | C | SER | 161 | 3.105 | −1.814 | 16.639 | 1.00 | 0.81 | 1SG | 1353 |
| ATOM | 1353 | O | SER | 161 | 2.002 | −2.036 | 17.135 | 1.00 | 0.81 | 1SG | 1354 |
| ATOM | 1354 | N | PHE | 162 | 3.477 | −2.321 | 15.453 | 1.00 | 0.92 | 1SG | 1355 |
| ATOM | 1355 | CA | PHE | 162 | 2.606 | −3.200 | 14.742 | 1.00 | 0.92 | 1SG | 1356 |
| ATOM | 1356 | CB | PHE | 162 | 3.011 | −3.362 | 13.263 | 1.00 | 0.92 | 1SG | 1357 |
| ATOM | 1357 | CG | PHE | 162 | 4.415 | −3.863 | 13.184 | 1.00 | 0.92 | 1SG | 1358 |
| ATOM | 1358 | CD1 | PHE | 162 | 5.470 | −2.980 | 13.249 | 1.00 | 0.92 | 1SG | 1359 |
| ATOM | 1359 | CD2 | PHE | 162 | 4.686 | −5.204 | 13.040 | 1.00 | 0.92 | 1SG | 1360 |
| ATOM | 1360 | CE1 | PHE | 162 | 6.770 | −3.422 | 13.175 | 1.00 | 0.92 | 1SG | 1361 |
| ATOM | 1361 | CE2 | PHE | 162 | 5.986 | −5.651 | 12.966 | 1.00 | 0.92 | 1SG | 1362 |
| ATOM | 1362 | CZ | PHE | 162 | 7.032 | −4.763 | 13.034 | 1.00 | 0.92 | 1SG | 1363 |
| ATOM | 1363 | C | PHE | 162 | 2.529 | −4.551 | 15.382 | 1.00 | 0.92 | 1SG | 1364 |
| ATOM | 1364 | O | PHE | 162 | 1.431 | −5.008 | 15.695 | 1.00 | 0.92 | 1SG | 1365 |
| ATOM | 1365 | N | LEU | 163 | 3.699 | −5.162 | 15.687 | 1.00 | 0.79 | 1SG | 1366 |
| ATOM | 1366 | CA | LEU | 163 | 3.782 | −6.536 | 16.116 | 1.00 | 0.79 | 1SG | 1367 |
| ATOM | 1367 | CB | LEU | 163 | 3.428 | −6.785 | 17.596 | 1.00 | 0.79 | 1SG | 1368 |
| ATOM | 1368 | CG | LEU | 163 | 4.520 | −6.337 | 18.591 | 1.00 | 0.79 | 1SG | 1369 |
| ATOM | 1369 | CD2 | LEU | 163 | 4.801 | −4.833 | 18.479 | 1.00 | 0.79 | 1SG | 1370 |
| ATOM | 1370 | CD1 | LEU | 163 | 5.794 | −7.193 | 18.460 | 1.00 | 0.79 | 1SG | 1371 |
| ATOM | 1371 | C | LEU | 163 | 2.898 | −7.377 | 15.245 | 1.00 | 0.79 | 1SG | 1372 |
| ATOM | 1372 | O | LEU | 163 | 1.698 | −7.508 | 15.474 | 1.00 | 0.79 | 1SG | 1373 |
| ATOM | 1373 | N | ALA | 164 | 3.505 | −8.010 | 14.223 | 1.00 | 0.48 | 1SG | 1374 |
| ATOM | 1374 | CA | ALA | 164 | 2.795 | −8.775 | 13.237 | 1.00 | 0.48 | 1SG | 1375 |
| ATOM | 1375 | CB | ALA | 164 | 3.730 | −9.472 | 12.233 | 1.00 | 0.48 | 1SG | 1376 |
| ATOM | 1376 | C | ALA | 164 | 2.003 | −9.852 | 13.903 | 1.00 | 0.48 | 1SG | 1377 |
| ATOM | 1377 | O | ALA | 164 | 0.898 | −10.170 | 13.464 | 1.00 | 0.48 | 1SG | 1378 |
| ATOM | 1378 | N | ALA | 165 | 2.545 | −10.442 | 14.984 | 1.00 | 0.32 | 1SG | 1379 |
| ATOM | 1379 | CA | ALA | 165 | 1.866 | −11.522 | 15.638 | 1.00 | 0.32 | 1SG | 1380 |
| ATOM | 1380 | CB | ALA | 165 | 2.578 | −11.980 | 16.922 | 1.00 | 0.32 | 1SG | 1381 |
| ATOM | 1381 | C | ALA | 165 | 0.501 | −11.057 | 16.024 | 1.00 | 0.32 | 1SG | 1382 |
| ATOM | 1382 | O | ALA | 165 | −0.491 | −11.725 | 15.734 | 1.00 | 0.32 | 1SG | 1383 |
| ATOM | 1383 | N | ALA | 166 | 0.409 | −9.877 | 16.662 | 1.00 | 0.43 | 1SG | 1384 |
| ATOM | 1384 | CA | ALA | 166 | −0.877 | −9.406 | 17.079 | 1.00 | 0.43 | 1SG | 1385 |
| ATOM | 1385 | CB | ALA | 166 | −0.976 | −9.146 | 18.593 | 1.00 | 0.43 | 1SG | 1386 |
| ATOM | 1386 | C | ALA | 166 | −1.139 | −8.112 | 16.386 | 1.00 | 0.43 | 1SG | 1387 |
| ATOM | 1387 | O | ALA | 166 | −0.780 | −7.922 | 15.225 | 1.00 | 0.43 | 1SG | 1388 |
| ATOM | 1388 | N | VAL | 167 | −1.857 | −7.204 | 17.070 | 1.00 | 0.50 | 1SG | 1389 |
| ATOM | 1389 | CA | VAL | 167 | −2.105 | −5.913 | 16.505 | 1.00 | 0.50 | 1SG | 1390 |
| ATOM | 1390 | CB | VAL | 167 | −3.196 | −5.975 | 15.474 | 1.00 | 0.50 | 1SG | 1391 |
| ATOM | 1391 | CG1 | VAL | 167 | −4.409 | −6.692 | 16.094 | 1.00 | 0.50 | 1SG | 1392 |
| ATOM | 1392 | CG2 | VAL | 167 | −3.495 | −4.552 | 14.983 | 1.00 | 0.50 | 1SG | 1393 |
| ATOM | 1393 | C | VAL | 167 | −2.492 | −5.011 | 17.640 | 1.00 | 0.50 | 1SG | 1394 |
| ATOM | 1394 | O | VAL | 167 | −3.661 | −4.869 | 17.985 | 1.00 | 0.50 | 1SG | 1395 |
| ATOM | 1395 | N | GLY | 168 | −1.480 | −4.391 | 18.272 | 1.00 | 0.30 | 1SG | 1396 |
| ATOM | 1396 | CA | GLY | 168 | −1.683 | −3.543 | 19.409 | 1.00 | 0.30 | 1SG | 1397 |
| ATOM | 1397 | C | GLY | 168 | −2.225 | −2.214 | 19.033 | 1.00 | 0.30 | 1SG | 1398 |
| ATOM | 1398 | O | GLY | 168 | −2.970 | −1.637 | 19.819 | 1.00 | 0.30 | 1SG | 1399 |
| ATOM | 1399 | N | ILE | 169 | −1.832 | −1.636 | 17.880 | 1.00 | 0.13 | 1SG | 1400 |
| ATOM | 1400 | CA | ILE | 169 | −2.406 | −0.355 | 17.583 | 1.00 | 0.13 | 1SG | 1401 |
| ATOM | 1401 | CB | ILE | 169 | −2.001 | 0.220 | 16.267 | 1.00 | 0.13 | 1SG | 1402 |
| ATOM | 1402 | CG2 | ILE | 169 | −2.896 | 1.439 | 15.996 | 1.00 | 0.13 | 1SG | 1403 |
| ATOM | 1403 | CG1 | ILE | 169 | −0.503 | 0.519 | 16.207 | 1.00 | 0.13 | 1SG | 1404 |
| ATOM | 1404 | CD1 | ILE | 169 | −0.051 | 0.801 | 14.778 | 1.00 | 0.13 | 1SG | 1405 |
| ATOM | 1405 | C | ILE | 169 | −3.870 | −0.579 | 17.469 | 1.00 | 0.13 | 1SG | 1406 |
| ATOM | 1406 | O | ILE | 169 | −4.673 | 0.124 | 18.079 | 1.00 | 0.13 | 1SG | 1407 |
| ATOM | 1407 | N | ALA | 170 | −4.256 | −1.607 | 16.705 | 1.00 | 0.19 | 1SG | 1408 |
| ATOM | 1408 | CA | ALA | 170 | −5.650 | −1.877 | 16.566 | 1.00 | 0.19 | 1SG | 1409 |
| ATOM | 1409 | CB | ALA | 170 | −5.950 | −3.042 | 15.603 | 1.00 | 0.19 | 1SG | 1410 |
| ATOM | 1410 | C | ALA | 170 | −6.154 | −2.250 | 17.916 | 1.00 | 0.19 | 1SG | 1411 |
| ATOM | 1411 | O | ALA | 170 | −7.259 | −1.885 | 18.312 | 1.00 | 0.19 | 1SG | 1412 |
| ATOM | 1412 | N | SER | 171 | −5.322 | −2.979 | 18.669 | 1.00 | 0.35 | 1SG | 1413 |
| ATOM | 1413 | CA | SER | 171 | −5.736 | −3.506 | 19.930 | 1.00 | 0.35 | 1SG | 1414 |
| ATOM | 1414 | CB | SER | 171 | −4.607 | −4.305 | 20.619 | 1.00 | 0.35 | 1SG | 1415 |
| ATOM | 1415 | OG | SER | 171 | −5.055 | −4.854 | 21.847 | 1.00 | 0.35 | 1SG | 1416 |
| ATOM | 1416 | C | SER | 171 | −6.142 | −2.376 | 20.823 | 1.00 | 0.35 | 1SG | 1417 |
| ATOM | 1417 | O | SER | 171 | −7.236 | −2.396 | 21.382 | 1.00 | 0.35 | 1SG | 1418 |
| ATOM | 1418 | N | CYS | 172 | −5.302 | −1.331 | 20.948 | 1.00 | 0.45 | 1SG | 1419 |
| ATOM | 1419 | CA | CYS | 172 | −5.676 | −0.272 | 21.838 | 1.00 | 0.45 | 1SG | 1420 |
| ATOM | 1420 | CB | CYS | 172 | −4.612 | 0.816 | 22.054 | 1.00 | 0.45 | 1SG | 1421 |
| ATOM | 1421 | SG | CYS | 172 | −3.685 | 0.582 | 23.605 | 1.00 | 0.45 | 1SG | 1422 |
| ATOM | 1422 | C | CYS | 172 | −6.924 | 0.398 | 21.382 | 1.00 | 0.45 | 1SG | 1423 |
| ATOM | 1423 | O | CYS | 172 | −7.832 | 0.630 | 22.180 | 1.00 | 0.45 | 1SG | 1424 |
| ATOM | 1424 | N | PHE | 173 | −7.018 | 0.724 | 20.084 | 1.00 | 0.48 | 1SG | 1425 |
| ATOM | 1425 | CA | PHE | 173 | −8.171 | 1.452 | 19.655 | 1.00 | 0.48 | 1SG | 1426 |
| ATOM | 1426 | CB | PHE | 173 | −8.095 | 1.941 | 18.190 | 1.00 | 0.48 | 1SG | 1427 |
| ATOM | 1427 | CG | PHE | 173 | −7.022 | 2.983 | 18.106 | 1.00 | 0.48 | 1SG | 1428 |
| ATOM | 1428 | CD1 | PHE | 173 | −7.299 | 4.304 | 18.376 | 1.00 | 0.48 | 1SG | 1429 |
| ATOM | 1429 | CD2 | PHE | 173 | −5.725 | 2.642 | 17.769 | 1.00 | 0.48 | 1SG | 1430 |
| ATOM | 1430 | CE1 | PHE | 173 | −6.291 | 5.245 | 18.300 | 1.00 | 0.48 | 1SG | 1431 |
| ATOM | 1431 | CE2 | PHE | 173 | −4.714 | 3.576 | 17.690 | 1.00 | 0.48 | 1SG | 1432 |
| ATOM | 1432 | CZ | PHE | 173 | −5.001 | 4.895 | 17.953 | 1.00 | 0.48 | 1SG | 1433 |
| ATOM | 1433 | C | PHE | 173 | −9.406 | 0.614 | 19.839 | 1.00 | 0.48 | 1SG | 1434 |
| ATOM | 1434 | O | PHE | 173 | −10.454 | 1.121 | 20.239 | 1.00 | 0.48 | 1SG | 1435 |
| ATOM | 1435 | N | SER | 174 | −9.326 | −0.699 | 19.545 | 1.00 | 0.41 | 1SG | 1436 |
| ATOM | 1436 | CA | SER | 174 | −10.500 | −1.517 | 19.671 | 1.00 | 0.41 | 1SG | 1437 |
| ATOM | 1437 | CB | SER | 174 | −10.320 | −2.942 | 19.128 | 1.00 | 0.41 | 1SG | 1438 |
| ATOM | 1438 | OG | SER | 174 | −11.523 | −3.680 | 19.283 | 1.00 | 0.41 | 1SG | 1439 |
| ATOM | 1439 | C | SER | 174 | −10.886 | −1.625 | 21.117 | 1.00 | 0.41 | 1SG | 1440 |
| ATOM | 1440 | O | SER | 174 | −12.066 | −1.522 | 21.454 | 1.00 | 0.41 | 1SG | 1441 |
| ATOM | 1441 | N | ASN | 175 | −9.890 | −1.814 | 22.003 | 1.00 | 0.39 | 1SG | 1442 |
| ATOM | 1442 | CA | ASN | 175 | −10.142 | −1.996 | 23.406 | 1.00 | 0.39 | 1SG | 1443 |
| ATOM | 1443 | CB | ASN | 175 | −8.878 | −2.430 | 24.172 | 1.00 | 0.39 | 1SG | 1444 |
| ATOM | 1444 | CG | ASN | 175 | −8.599 | −3.867 | 23.739 | 1.00 | 0.39 | 1SG | 1445 |
| ATOM | 1445 | OD1 | ASN | 175 | −9.511 | −4.691 | 23.689 | 1.00 | 0.39 | 1SG | 1446 |
| ATOM | 1446 | ND2 | ASN | 175 | −7.336 | −4.161 | −23.327 | 1.00 | 0.39 | 1SG | 1447 |
| ATOM | 1447 | C | ASN | 175 | −10.740 | −0.755 | 23.997 | 1.00 | 0.39 | 1SG | 1448 |
| ATOM | 1448 | O | ASN | 175 | −11.596 | −0.825 | 24.881 | 1.00 | 0.39 | 1SG | 1449 |
| ATOM | 1449 | N | VAL | 176 | −10.300 | 0.426 | 23.531 | 1.00 | 0.68 | 1SG | 1450 |
| ATOM | 1450 | CA | VAL | 176 | −10.891 | 1.647 | 23.996 | 1.00 | 0.68 | 1SG | 1451 |
| ATOM | 1451 | CB | VAL | 176 | −10.022 | 2.839 | 23.706 | 1.00 | 0.68 | 1SG | 1452 |
| ATOM | 1452 | CG1 | VAL | 176 | −8.723 | 2.710 | 24.528 | 1.00 | 0.68 | 1SG | 1453 |
| ATOM | 1453 | CG2 | VAL | 176 | −9.782 | 2.918 | 22.185 | 1.00 | 0.68 | 1SG | 1454 |
| ATOM | 1454 | C | VAL | 176 | −12.166 | 1.823 | 23.214 | 1.00 | 0.68 | 1SG | 1455 |
| ATOM | 1455 | O | VAL | 176 | −12.549 | 2.927 | 22.848 | 1.00 | 0.68 | 1SG | 1456 |
| ATOM | 1456 | N | PHE | 177 | −12.923 | 0.738 | 23.017 | 1.00 | 1.02 | 1SG | 1457 |
| ATOM | 1457 | CA | PHE | 177 | −14.061 | 0.696 | 22.145 | 1.00 | 1.02 | 1SG | 1458 |
| ATOM | 1458 | CB | PHE | 177 | −14.759 | −0.675 | 22.358 | 1.00 | 1.02 | 1SG | 1459 |
| ATOM | 1459 | CG | PHE | 177 | −15.714 | −1.065 | 21.285 | 1.00 | 1.02 | 1SG | 1460 |
| ATOM | 1460 | CD1 | PHE | 177 | −16.995 | −0.575 | 21.279 | 1.00 | 1.02 | 1SG | 1461 |
| ATOM | 1461 | CD2 | PHE | 177 | −15.355 | −1.962 | 20.312 | 1.00 | 1.02 | 1SG | 1462 |
| ATOM | 1462 | CE1 | PHE | 177 | −17.893 | −0.931 | 20.306 | 1.00 | 1.02 | 1SG | 1463 |
| ATOM | 1463 | CE2 | PHE | 177 | −16.245 | −2.331 | 19.326 | 1.00 | 1.02 | 1SG | 1464 |
| ATOM | 1464 | CZ | PHE | 177 | −17.520 | −1.822 | 19.323 | 1.00 | 1.02 | 1SG | 1465 |
| ATOM | 1465 | C | PHE | 177 | −15.031 | 1.817 | 22.443 | 1.00 | 1.02 | 1SG | 1466 |
| ATOM | 1466 | O | PHE | 177 | −15.117 | 2.754 | 21.651 | 1.00 | 1.02 | 1SG | 1467 |
| ATOM | 1467 | N | VAL | 178 | −15.742 | 1.822 | 23.597 | 1.00 | 1.02 | 1SG | 1468 |
| ATOM | 1468 | CA | VAL | 178 | −16.654 | 2.928 | 23.790 | 1.00 | 1.02 | 1SG | 1469 |
| ATOM | 1469 | CB | VAL | 178 | −17.618 | 3.193 | 22.661 | 1.00 | 1.02 | 1SG | 1470 |
| ATOM | 1470 | CG1 | VAL | 178 | −18.280 | 1.904 | 22.159 | 1.00 | 1.02 | 1SG | 1471 |
| ATOM | 1471 | CG2 | VAL | 178 | −18.673 | 4.161 | 23.216 | 1.00 | 1.02 | 1SG | 1472 |
| ATOM | 1472 | C | VAL | 178 | −17.516 | 2.801 | 25.009 | 1.00 | 1.02 | 1SG | 1473 |
| ATOM | 1473 | O | VAL | 178 | −17.324 | 3.543 | 25.975 | 1.00 | 1.02 | 1SG | 1474 |
| ATOM | 1474 | N | ALA | 179 | −18.507 | 1.880 | 24.967 | 1.00 | 0.78 | 1SG | 1475 |
| ATOM | 1475 | CA | ALA | 179 | −19.511 | 1.729 | 25.988 | 1.00 | 0.78 | 1SG | 1476 |
| ATOM | 1476 | CB | ALA | 179 | −20.678 | 0.818 | 25.564 | 1.00 | 0.78 | 1SG | 1477 |
| ATOM | 1477 | C | ALA | 179 | −18.916 | 1.156 | 27.231 | 1.00 | 0.78 | 1SG | 1478 |
| ATOM | 1478 | O | ALA | 179 | −19.589 | 0.467 | 28.001 | 1.00 | 0.78 | 1SG | 1479 |
| ATOM | 1479 | N | SER | 180 | −17.623 | 1.418 | 27.457 | 1.00 | 0.71 | 1SG | 1480 |
| ATOM | 1480 | CA | SER | 180 | −16.996 | 1.027 | 28.669 | 1.00 | 0.71 | 1SG | 1481 |
| ATOM | 1481 | CB | SER | 180 | −15.922 | −0.053 | 28.475 | 1.00 | 0.71 | 1SG | 1482 |
| ATOM | 1482 | OG | SER | 180 | −14.916 | 0.398 | 27.583 | 1.00 | 0.71 | 1SG | 1483 |
| ATOM | 1483 | C | SER | 180 | −16.337 | 2.254 | 29.202 | 1.00 | 0.71 | 1SG | 1484 |
| ATOM | 1484 | O | SER | 180 | −15.416 | 2.156 | 30.009 | 1.00 | 0.71 | 1SG | 1485 |
| ATOM | 1485 | N | GLN | 181 | −16.827 | 3.443 | 28.785 | 1.00 | 0.96 | 1SG | 1486 |
| ATOM | 1486 | CA | GLN | 181 | −16.300 | 4.689 | 29.274 | 1.00 | 0.96 | 1SG | 1487 |
| ATOM | 1487 | CB | GLN | 181 | −16.026 | 4.695 | 30.800 | 1.00 | 0.96 | 1SG | 1488 |
| ATOM | 1488 | CG | GLN | 181 | −15.272 | 5.926 | 31.319 | 1.00 | 0.96 | 1SG | 1489 |
| ATOM | 1489 | CD | GLN | 181 | −14.625 | 5.554 | 32.660 | 1.00 | 0.96 | 1SG | 1490 |
| ATOM | 1490 | OE1 | GLN | 181 | −13.943 | 4.532 | 32.767 | 1.00 | 0.96 | 1SG | 1491 |
| ATOM | 1491 | NE2 | GLN | 181 | −14.836 | 6.396 | 33.709 | 1.00 | 0.96 | 1SG | 1492 |
| ATOM | 1492 | C | GLN | 181 | −14.984 | 4.978 | 28.628 | 1.00 | 0.96 | 1SG | 1493 |
| ATOM | 1493 | O | GLN | 181 | −14.527 | 6.121 | 28.678 | 1.00 | 0.96 | 1SG | 1494 |
| ATOM | 1494 | N | LEU | 182 | −14.318 | 3.973 | 28.016 | 1.00 | 1.07 | 1SG | 1495 |
| ATOM | 1495 | CA | LEU | 182 | −12.995 | 4.310 | 27.583 | 1.00 | 1.07 | 1SG | 1496 |
| ATOM | 1496 | CB | LEU | 182 | −12.204 | 3.103 | 27.052 | 1.00 | 1.07 | 1SG | 1497 |
| ATOM | 1497 | CG | LEU | 182 | −11.728 | 2.162 | 28.176 | 1.00 | 1.07 | 1SG | 1498 |
| ATOM | 1498 | CD2 | LEU | 182 | −12.909 | 1.677 | 29.023 | 1.00 | 1.07 | 1SG | 1499 |
| ATOM | 1499 | CD1 | LEU | 182 | −10.656 | 2.822 | 29.056 | 1.00 | 1.07 | 1SG | 1500 |
| ATOM | 1500 | C | LEU | 182 | −12.948 | 5.406 | 26.558 | 1.00 | 1.07 | 1SG | 1501 |
| ATOM | 1501 | O | LEU | 182 | −12.641 | 6.536 | 26.919 | 1.00 | 1.07 | 1SG | 1502 |
| ATOM | 1502 | N | GLU | 183 | −13.273 | 5.145 | 25.273 | 1.00 | 0.77 | 1SG | 1503 |
| ATOM | 1503 | CA | GLU | 183 | −13.112 | 6.210 | 24.309 | 1.00 | 0.77 | 1SG | 1504 |
| ATOM | 1504 | CB | GLU | 183 | −12.881 | 5.754 | 22.852 | 1.00 | 0.77 | 1SG | 1505 |
| ATOM | 1505 | CG | GLU | 183 | −11.440 | 5.372 | 22.540 | 1.00 | 0.77 | 1SG | 1506 |
| ATOM | 1506 | CD | GLU | 183 | −10.528 | 6.430 | 23.105 | 1.00 | 0.77 | 1SG | 1507 |
| ATOM | 1507 | OE1 | GLU | 183 | −10.952 | 7.616 | 23.155 | 1.00 | 0.77 | 1SG | 1508 |
| ATOM | 1508 | OE2 | GLU | 183 | −9.395 | 6.070 | 23.501 | 1.00 | 0.77 | 1SG | 1509 |
| ATOM | 1509 | C | GLU | 183 | −14.231 | 7.197 | 24.231 | 1.00 | 0.77 | 1SG | 1510 |
| ATOM | 1510 | O | GLU | 183 | −14.017 | 8.404 | 24.346 | 1.00 | 0.77 | 1SG | 1511 |
| ATOM | 1511 | N | SER | 184 | −15.463 | 6.704 | 24.011 | 1.00 | 0.43 | 1SG | 1512 |
| ATOM | 1512 | CA | SER | 184 | −16.513 | 7.629 | 23.703 | 1.00 | 0.43 | 1SG | 1513 |
| ATOM | 1513 | CB | SER | 184 | −17.807 | 6.969 | 23.203 | 1.00 | 0.43 | 1SG | 1514 |
| ATOM | 1514 | OG | SER | 184 | −18.781 | 7.963 | 22.918 | 1.00 | 0.43 | 1SG | 1515 |
| ATOM | 1515 | C | SER | 184 | −16.875 | 8.462 | 24.879 | 1.00 | 0.43 | 1SG | 1516 |
| ATOM | 1516 | O | SER | 184 | −17.026 | 9.676 | 24.759 | 1.00 | 0.43 | 1SG | 1517 |
| ATOM | 1517 | N | VAL | 185 | −17.025 | 7.833 | 26.053 | 1.00 | 0.33 | 1SG | 1518 |
| ATOM | 1518 | CA | VAL | 185 | −17.519 | 8.578 | 27.166 | 1.00 | 0.33 | 1SG | 1519 |
| ATOM | 1519 | CB | VAL | 185 | −17.831 | 7.736 | 28.363 | 1.00 | 0.33 | 1SG | 1520 |
| ATOM | 1520 | CG1 | VAL | 185 | −18.279 | 8.679 | 29.497 | 1.00 | 0.33 | 1SG | 1521 |
| ATOM | 1521 | CG2 | VAL | 185 | −18.906 | 6.711 | 27.965 | 1.00 | 0.33 | 1SG | 1522 |
| ATOM | 1522 | C | VAL | 185 | −16.561 | 9.647 | 27.584 | 1.00 | 0.33 | 1SG | 1523 |
| ATOM | 1523 | O | VAL | 185 | −16.974 | 10.775 | 27.855 | 1.00 | 0.33 | 1SG | 1524 |
| ATOM | 1524 | N | VAL | 186 | −15.248 | 9.349 | 27.619 | 1.00 | 0.43 | 1SG | 1525 |
| ATOM | 1525 | CA | VAL | 186 | −14.361 | 10.338 | 28.157 | 1.00 | 0.43 | 1SG | 1526 |
| ATOM | 1526 | CB | VAL | 186 | −13.013 | 9.792 | 28.523 | 1.00 | 0.43 | 1SG | 1527 |
| ATOM | 1527 | CG1 | VAL | 186 | −12.345 | 9.212 | 27.275 | 1.00 | 0.43 | 1SG | 1528 |
| ATOM | 1528 | CG2 | VAL | 186 | −12.209 | 10.907 | 29.205 | 1.00 | 0.43 | 1SG | 1529 |
| ATOM | 1529 | C | VAL | 186 | −14.208 | 11.488 | 27.208 | 1.00 | 0.43 | 1SG | 1530 |
| ATOM | 1530 | O | VAL | 186 | −13.473 | 11.449 | 26.222 | 1.00 | 0.43 | 1SG | 1531 |
| ATOM | 1531 | N | TYR | 187 | −14.937 | 12.574 | 27.508 | 1.00 | 0.42 | 1SG | 1532 |
| ATOM | 1532 | CA | TYR | 187 | −14.905 | 13.777 | 26.739 | 1.00 | 0.42 | 1SG | 1533 |
| ATOM | 1533 | CB | TYR | 187 | −15.969 | 14.773 | 27.241 | 1.00 | 0.42 | 1SG | 1534 |
| ATOM | 1534 | CG | TYR | 187 | −16.035 | 15.947 | 26.329 | 1.00 | 0.42 | 1SG | 1535 |
| ATOM | 1535 | CD1 | TYR | 187 | −16.837 | 15.913 | 25.210 | 1.00 | 0.42 | 1SG | 1536 |
| ATOM | 1536 | CD2 | TYR | 187 | −15.308 | 17.083 | 26.595 | 1.00 | 0.42 | 1SG | 1537 |
| ATOM | 1537 | CE1 | TYR | 187 | −16.907 | 16.994 | 24.364 | 1.00 | 0.42 | 1SG | 1538 |
| ATOM | 1538 | CE2 | TYR | 187 | −15.374 | 18.167 | 25.752 | 1.00 | 0.42 | 1SG | 1539 |
| ATOM | 1539 | CZ | TYR | 187 | −16.173 | 18.123 | 24.635 | 1.00 | 0.42 | 1SG | 1540 |
| ATOM | 1540 | OH | TYR | 187 | −16.241 | 19.235 | 23.769 | 1.00 | 0.42 | 1SG | 1541 |
| ATOM | 1541 | C | TYR | 187 | −13.557 | 14.396 | 26.941 | 1.00 | 0.42 | 1SG | 1542 |
| ATOM | 1542 | O | TYR | 187 | −12.938 | 14.905 | 26.009 | 1.00 | 0.42 | 1SG | 1543 |
| ATOM | 1543 | N | ALA | 188 | −13.066 | 14.338 | 28.193 | 1.00 | 0.24 | 1SG | 1544 |
| ATOM | 1544 | CA | ALA | 188 | −11.838 | 14.960 | 28.579 | 1.00 | 0.24 | 1SG | 1545 |
| ATOM | 1545 | CB | ALA | 188 | −11.536 | 14.810 | 30.077 | 1.00 | 0.24 | 1SG | 1546 |
| ATOM | 1546 | C | ALA | 188 | −10.713 | 14.349 | 27.829 | 1.00 | 0.24 | 1SG | 1547 |
| ATOM | 1547 | O | ALA | 188 | −9.796 | 15.052 | 27.407 | 1.00 | 0.24 | 1SG | 1548 |
| ATOM | 1548 | N | SER | 189 | −10.727 | 13.019 | 27.629 | 1.00 | 0.28 | 1SG | 1549 |
| ATOM | 1549 | CA | SER | 189 | −9.573 | 12.536 | 26.948 | 1.00 | 0.28 | 1SG | 1550 |
| ATOM | 1550 | CB | SER | 189 | −9.239 | 11.038 | 27.122 | 1.00 | 0.28 | 1SG | 1551 |
| ATOM | 1551 | OG | SER | 189 | −9.989 | 10.228 | 26.231 | 1.00 | 0.28 | 1SG | 1552 |
| ATOM | 1552 | C | SER | 189 | −9.780 | 12.819 | 25.511 | 1.00 | 0.28 | 1SG | 1553 |
| ATOM | 1553 | O | SER | 189 | −10.685 | 12.292 | 24.863 | 1.00 | 0.28 | 1SG | 1554 |
| ATOM | 1554 | N | TRP | 190 | −8.956 | 13.739 | 24.998 | 1.00 | 0.46 | 1SG | 1555 |
| ATOM | 1555 | CA | TRP | 190 | −8.994 | 14.025 | 23.610 | 1.00 | 0.46 | 1SG | 1556 |
| ATOM | 1556 | CB | TRP | 190 | −7.918 | 15.099 | 23.319 | 1.00 | 0.46 | 1SG | 1557 |
| ATOM | 1557 | CG | TRP | 190 | −7.801 | 15.788 | 21.971 | 1.00 | 0.46 | 1SG | 1558 |
| ATOM | 1558 | CD2 | TRP | 190 | −8.487 | 16.999 | 21.603 | 1.00 | 0.46 | 1SG | 1559 |
| ATOM | 1559 | CD1 | TRP | 190 | −6.986 | 15.488 | 20.925 | 1.00 | 0.46 | 1SG | 1560 |
| ATOM | 1560 | NE1 | TRP | 190 | −7.118 | 16.422 | 19.928 | 1.00 | 0.46 | 1SG | 1561 |
| ATOM | 1561 | CE2 | TRP | 190 | −8.036 | 17.362 | 20.332 | 1.00 | 0.46 | 1SG | 1562 |
| ATOM | 1562 | CE3 | TRP | 190 | −9.404 | 17.758 | 22.270 | 1.00 | 0.46 | 1SG | 1563 |
| ATOM | 1563 | CZ2 | TRP | 190 | −8.497 | 18.488 | 19.710 | 1.00 | 0.46 | 1SG | 1564 |
| ATOM | 1564 | CZ3 | TRP | 190 | −9.874 | 18.887 | 21.634 | 1.00 | 0.46 | 1SG | 1565 |
| ATOM | 1565 | CH2 | TRP | 190 | −9.430 | 19.247 | 20.378 | 1.00 | 0.46 | 1SG | 1566 |
| ATOM | 1566 | C | TRP | 190 | −8.619 | 12.695 | 23.055 | 1.00 | 0.46 | 1SG | 1567 |
| ATOM | 1567 | O | TRP | 190 | −9.370 | 12.065 | 22.316 | 1.00 | 0.46 | 1SG | 1568 |
| ATOM | 1568 | N | SER | 191 | −7.471 | 12.167 | 23.509 | 1.00 | 0.70 | 1SG | 1569 |
| ATOM | 1569 | CA | SER | 191 | −7.056 | 10.897 | 23.010 | 1.00 | 0.70 | 1SG | 1570 |
| ATOM | 1570 | CB | SER | 191 | −5.608 | 10.891 | 22.502 | 1.00 | 0.70 | 1SG | 1571 |
| ATOM | 1571 | OG | SER | 191 | −5.405 | 11.927 | 21.554 | 1.00 | 0.70 | 1SG | 1572 |
| ATOM | 1572 | C | SER | 191 | −7.043 | 9.957 | 24.163 | 1.00 | 0.70 | 1SG | 1573 |
| ATOM | 1573 | O | SER | 191 | −6.057 | 9.899 | 24.894 | 1.00 | 0.70 | 1SG | 1574 |
| ATOM | 1574 | N | ARG | 192 | −8.121 | 9.187 | 24.376 | 1.00 | 0.88 | 1SG | 1575 |
| ATOM | 1575 | CA | ARG | 192 | −7.980 | 8.272 | 25.460 | 1.00 | 0.88 | 1SG | 1576 |
| ATOM | 1576 | CB | ARG | 192 | −9.257 | 7.500 | 25.834 | 1.00 | 0.88 | 1SG | 1577 |
| ATOM | 1577 | CG | ARG | 192 | −9.276 | 7.029 | 27.297 | 1.00 | 0.88 | 1SG | 1578 |
| ATOM | 1578 | CD | ARG | 192 | −7.996 | 7.328 | 28.092 | 1.00 | 0.88 | 1SG | 1579 |
| ATOM | 1579 | NE | ARG | 192 | −8.178 | 8.646 | 28.773 | 1.00 | 0.88 | 1SG | 1580 |
| ATOM | 1580 | CZ | ARG | 192 | −8.812 | 8.723 | 29.980 | 1.00 | 0.88 | 1SG | 1581 |
| ATOM | 1581 | NH1 | ARG | 192 | −9.290 | 7.585 | 30.572 | 1.00 | 0.88 | 1SG | 1582 |
| ATOM | 1582 | NH2 | ARG | 192 | −8.958 | 9.925 | 30.606 | 1.00 | 0.88 | 1SG | 1583 |
| ATOM | 1583 | C | ARG | 192 | −6.946 | 7.301 | 24.990 | 1.00 | 0.88 | 1SG | 1584 |
| ATOM | 1584 | O | ARG | 192 | −6.124 | 6.824 | 25.773 | 1.00 | 0.88 | 1SG | 1585 |
| ATOM | 1585 | N | VAL | 193 | −6.955 | 6.979 | 23.678 | 1.00 | 0.78 | 1SG | 1586 |
| ATOM | 1586 | CA | VAL | 193 | −5.931 | 6.090 | 23.225 | 1.00 | 0.78 | 1SG | 1587 |
| ATOM | 1587 | CB | VAL | 193 | −6.396 | 4.838 | 22.519 | 1.00 | 0.78 | 1SG | 1588 |
| ATOM | 1588 | CG1 | VAL | 193 | −7.074 | 5.180 | 21.187 | 1.00 | 0.78 | 1SG | 1589 |
| ATOM | 1589 | CG2 | VAL | 193 | −5.183 | 3.905 | 22.362 | 1.00 | 0.78 | 1SG | 1590 |
| ATOM | 1590 | C | VAL | 193 | −4.986 | 6.867 | 22.375 | 1.00 | 0.78 | 1SG | 1591 |
| ATOM | 1591 | O | VAL | 193 | −5.233 | 7.199 | 21.212 | 1.00 | 0.78 | 1SG | 1592 |
| ATOM | 1592 | N | GLN | 194 | −3.851 | 7.172 | 23.018 | 1.00 | 0.63 | 1SG | 1593 |
| ATOM | 1593 | CA | GLN | 194 | −2.765 | 7.965 | 22.553 | 1.00 | 0.63 | 1SG | 1594 |
| ATOM | 1594 | CB | GLN | 194 | −2.564 | 9.133 | 23.537 | 1.00 | 0.63 | 1SG | 1595 |
| ATOM | 1595 | CG | GLN | 194 | −2.111 | 8.706 | 24.940 | 1.00 | 0.63 | 1SG | 1596 |
| ATOM | 1596 | CD | GLN | 194 | −0.586 | 8.681 | 24.977 | 1.00 | 0.63 | 1SG | 1597 |
| ATOM | 1597 | OE1 | GLN | 194 | 0.080 | 9.622 | 24.547 | 1.00 | 0.63 | 1SG | 1598 |
| ATOM | 1598 | NE2 | GLN | 194 | −0.014 | 7.577 | 25.524 | 1.00 | 0.63 | 1SG | 1599 |
| ATOM | 1599 | C | GLN | 194 | −1.569 | 7.068 | 22.684 | 1.00 | 0.63 | 1SG | 1600 |
| ATOM | 1600 | O | GLN | 194 | −1.494 | 6.300 | 23.639 | 1.00 | 0.63 | 1SG | 1601 |
| ATOM | 1601 | N | ALA | 195 | −0.625 | 7.089 | 21.719 | 1.00 | 0.48 | 1SG | 1602 |
| ATOM | 1602 | CA | ALA | 195 | 0.531 | 6.240 | 21.829 | 1.00 | 0.48 | 1SG | 1603 |
| ATOM | 1603 | CB | ALA | 195 | 1.405 | 6.262 | 20.565 | 1.00 | 0.48 | 1SG | 1604 |
| ATOM | 1604 | C | ALA | 195 | 1.428 | 6.568 | 23.023 | 1.00 | 0.48 | 1SG | 1605 |
| ATOM | 1605 | O | ALA | 195 | 1.397 | 5.820 | 23.998 | 1.00 | 0.48 | 1SG | 1606 |
| ATOM | 1606 | N | ASP | 196 | 2.217 | 7.690 | 23.018 | 1.00 | 0.46 | 1SG | 1607 |
| ATOM | 1607 | CA | ASP | 196 | 3.107 | 8.035 | 24.126 | 1.00 | 0.46 | 1SG | 1608 |
| ATOM | 1608 | CB | ASP | 196 | 3.858 | 6.813 | 24.694 | 1.00 | 0.46 | 1SG | 1609 |
| ATOM | 1609 | CG | ASP | 196 | 4.624 | 7.219 | 25.945 | 1.00 | 0.46 | 1SG | 1610 |
| ATOM | 1610 | OD1 | ASP | 196 | 4.524 | 8.407 | 26.346 | 1.00 | 0.46 | 1SG | 1611 |
| ATOM | 1611 | OD2 | ASP | 196 | 5.323 | 6.337 | 26.519 | 1.00 | 0.46 | 1SG | 1612 |
| ATOM | 1612 | C | ASP | 196 | 4.180 | 9.021 | 23.678 | 1.00 | 0.46 | 1SG | 1613 |
| ATOM | 1613 | O | ASP | 196 | 4.875 | 8.782 | 22.690 | 1.00 | 0.46 | 1SG | 1614 |
| ATOM | 1614 | N | LEU | 197 | 4.347 | 10.161 | 24.406 | 1.00 | 0.64 | 1SG | 1615 |
| ATOM | 1615 | CA | LEU | 197 | 5.324 | 11.191 | 24.101 | 1.00 | 0.64 | 1SG | 1616 |
| ATOM | 1616 | CB | LEU | 197 | 5.025 | 12.551 | 24.756 | 1.00 | 0.64 | 1SG | 1617 |
| ATOM | 1617 | CG | LEU | 197 | 3.728 | 13.222 | 24.277 | 1.00 | 0.64 | 1SG | 1618 |
| ATOM | 1618 | CD2 | LEU | 197 | 3.766 | 14.729 | 24.565 | 1.00 | 0.64 | 1SG | 1619 |
| ATOM | 1619 | CD1 | LEU | 197 | 2.478 | 12.504 | 24.813 | 1.00 | 0.64 | 1SG | 1620 |
| ATOM | 1620 | C | LEU | 197 | 6.717 | 10.846 | 24.523 | 1.00 | 0.64 | 1SG | 1621 |
| ATOM | 1621 | O | LEU | 197 | 7.661 | 11.007 | 23.750 | 1.00 | 0.64 | 1SG | 1622 |
| ATOM | 1622 | N | ASN | 198 | 6.893 | 10.378 | 25.774 | 1.00 | 0.61 | 1SG | 1623 |
| ATOM | 1623 | CA | ASN | 198 | 8.226 | 10.139 | 26.250 | 1.00 | 0.61 | 1SG | 1624 |
| ATOM | 1624 | CB | ASN | 198 | 8.457 | 10.650 | 27.677 | 1.00 | 0.61 | 1SG | 1625 |
| ATOM | 1625 | CG | ASN | 198 | 9.960 | 10.744 | 27.869 | 1.00 | 0.61 | 1SG | 1626 |
| ATOM | 1626 | OD1 | ASN | 198 | 10.643 | 11.528 | 27.211 | 1.00 | 0.61 | 1SG | 1627 |
| ATOM | 1627 | ND2 | ASN | 198 | 10.496 | 9.915 | 28.802 | 1.00 | 0.61 | 1SG | 1628 |
| ATOM | 1628 | C | ASN | 198 | 8.431 | 8.667 | 26.268 | 1.00 | 0.61 | 1SG | 1629 |
| ATOM | 1629 | O | ASN | 198 | 8.506 | 8.039 | 27.322 | 1.00 | 0.61 | 1SG | 1630 |
| ATOM | 1630 | N | CYS | 199 | 8.536 | 8.104 | 25.059 | 1.00 | 0.53 | 1SG | 1631 |
| ATOM | 1631 | CA | CYS | 199 | 8.686 | 6.708 | 24.804 | 1.00 | 0.53 | 1SG | 1632 |
| ATOM | 1632 | CB | CYS | 199 | 8.506 | 6.347 | 23.324 | 1.00 | 0.53 | 1SG | 1633 |
| ATOM | 1633 | SG | CYS | 199 | 6.815 | 6.638 | 22.722 | 1.00 | 0.53 | 1SG | 1634 |
| ATOM | 1634 | C | CYS | 199 | 10.047 | 6.236 | 25.193 | 1.00 | 0.53 | 1SG | 1635 |
| ATOM | 1635 | O | CYS | 199 | 10.242 | 5.032 | 25.339 | 1.00 | 0.53 | 1SG | 1636 |
| ATOM | 1636 | N | MET | 200 | 11.017 | 7.164 | 25.327 | 1.00 | 0.58 | 1SG | 1637 |
| ATOM | 1637 | CA | MET | 200 | 12.401 | 6.840 | 25.547 | 1.00 | 0.58 | 1SG | 1638 |
| ATOM | 1638 | CB | MET | 200 | 13.269 | 8.029 | 25.970. | 1.00 | 0.58 | 1SG | 1639 |
| ATOM | 1639 | CG | MET | 200 | 14.752 | 7.728 | 25.786 | 1.00 | 0.58 | 1SG | 1640 |
| ATOM | 1640 | SD | MET | 200 | 15.815 | 9.197 | 25.848 | 1.00 | 0.58 | 1SG | 1641 |
| ATOM | 1641 | CE | MET | 200 | 15.256 | 9.883 | 24.256 | 1.00 | 0.58 | 1SG | 1642 |
| ATOM | 1642 | C | MET | 200 | 12.601 | 5.729 | 26.522 | 1.00 | 0.58 | 1SG | 1643 |
| ATOM | 1643 | O | MET | 200 | 11.928 | 5.632 | 27.547 | 1.00 | 0.58 | 1SG | 1644 |
| ATOM | 1644 | N | GLN | 201 | 13.549 | 4.834 | 26.168 | 1.00 | 0.47 | 1SG | 1645 |
| ATOM | 1645 | CA | GLN | 201 | 13.910 | 3.644 | 26.893 | 1.00 | 0.47 | 1SG | 1646 |
| ATOM | 1646 | CB | GLN | 201 | 15.056 | 2.896 | 26.183 | 1.00 | 0.47 | 1SG | 1647 |
| ATOM | 1647 | CG | GLN | 201 | 14.761 | 2.470 | 24.742 | 1.00 | 0.47 | 1SG | 1648 |
| ATOM | 1648 | CD | GLN | 201 | 14.412 | 0.990 | 24.729 | 1.00 | 0.47 | 1SG | 1649 |
| ATOM | 1649 | OE1 | GLN | 201 | 14.583 | 0.317 | 23.712 | 1.00 | 0.47 | 1SG | 1650 |
| ATOM | 1650 | NE2 | GLN | 201 | 13.926 | 0.463 | 25.884 | 1.00 | 0.47 | 1SG | 1651 |
| ATOM | 1651 | C | GLN | 201 | 14.455 | 3.987 | 28.248 | 1.00 | 0.47 | 1SG | 1652 |
| ATOM | 1652 | O | GLN | 201 | 13.902 | 3.578 | 29.268 | 1.00 | 0.47 | 1SG | 1653 |
| ATOM | 1653 | N | ASP | 202 | 15.552 | 4.772 | 28.278 | 1.00 | 0.28 | 1SG | 1654 |
| ATOM | 1654 | CA | ASP | 202 | 16.228 | 5.111 | 29.501 | 1.00 | 0.28 | 1SG | 1655 |
| ATOM | 1655 | CB | ASP | 202 | 17.542 | 5.886 | 29.272 | 1.00 | 0.28 | 1SG | 1656 |
| ATOM | 1656 | CG | ASP | 202 | 18.608 | 4.945 | 28.721 | 1.00 | 0.28 | 1SG | 1657 |
| ATOM | 1657 | OD1 | ASP | 202 | 18.466 | 3.706 | 28.899 | 1.00 | 0.28 | 1SG | 1658 |
| ATOM | 1658 | OD2 | ASP | 202 | 19.587 | 5.463 | 28.118 | 1.00 | 0.28 | 1SG | 1659 |
| ATOM | 1659 | C | ASP | 202 | 15.344 | 6.002 | 30.292 | 1.00 | 0.28 | 1SG | 1660 |
| ATOM | 1660 | O | ASP | 202 | 15.257 | 5.908 | 31.518 | 1.00 | 0.28 | 1SG | 1661 |
| ATOM | 1661 | N | LEU | 203 | 14.622 | 6.872 | 29.573 | 1.00 | 0.41 | 1SG | 1662 |
| ATOM | 1662 | CA | LEU | 203 | 13.843 | 7.876 | 30.208 | 1.00 | 0.41 | 1SG | 1663 |
| ATOM | 1663 | CB | LEU | 203 | 13.103 | 8.801 | 29.223 | 1.00 | 0.41 | 1SG | 1664 |
| ATOM | 1664 | CG | LEU | 203 | 14.064 | 9.626 | 28.338 | 1.00 | 0.41 | 1SG | 1665 |
| ATOM | 1665 | CD2 | LEU | 203 | 15.219 | 10.228 | 29.162 | 1.00 | 0.41 | 1SG | 1666 |
| ATOM | 1666 | CD1 | LEU | 203 | 13.304 | 10.665 | 27.496 | 1.00 | 0.41 | 1SG | 1667 |
| ATOM | 1667 | C | LEU | 203 | 12.867 | 7.207 | 31.090 | 1.00 | 0.41 | 1SG | 1668 |
| ATOM | 1668 | O | LEU | 203 | 12.400 | 7.810 | 32.053 | 1.00 | 0.41 | 1SG | 1669 |
| ATOM | 1669 | N | TYR | 204 | 12.495 | 5.964 | 30.738 | 1.00 | 0.76 | 1SG | 1670 |
| ATOM | 1670 | CA | TYR | 204 | 11.573 | 5.224 | 31.536 | 1.00 | 0.76 | 1SG | 1671 |
| ATOM | 1671 | CB | TYR | 204 | 11.836 | 5.348 | 33.059 | 1.00 | 0.76 | 1SG | 1672 |
| ATOM | 1672 | CG | TYR | 204 | 10.989 | 4.453 | 33.905 | 1.00 | 0.76 | 1SG | 1673 |
| ATOM | 1673 | CD1 | TYR | 204 | 11.430 | 3.194 | 34.243 | 1.00 | 0.76 | 1SG | 1674 |
| ATOM | 1674 | CD2 | TYR | 204 | 9.775 | 4.872 | 34.399 | 1.00 | 0.76 | 1SG | 1675 |
| ATOM | 1675 | CE1 | TYR | 204 | 10.664 | 2.373 | 35.035 | 1.00 | 0.76 | 1SG | 1676 |
| ATOM | 1676 | CE2 | TYR | 204 | 9.004 | 4.058 | 35.190 | 1.00 | 0.76 | 1SG | 1677 |
| ATOM | 1677 | CZ | TYR | 204 | 9.450 | 2.801 | 35.511 | 1.00 | 0.76 | 1SG | 1678 |
| ATOM | 1678 | OH | TYR | 204 | 8.665 | 1.960 | 36.327 | 1.00 | 0.76 | 1SG | 1679 |
| ATOM | 1679 | C | TYR | 204 | 10.261 | 5.787 | 31.143 | 1.00 | 0.76 | 1SG | 1680 |
| ATOM | 1680 | O | TYR | 204 | 9.894 | 5.683 | 29.974 | 1.00 | 0.76 | 1SG | 1681 |
| ATOM | 1681 | N | GLN | 205 | 9.563 | 6.451 | 32.086 | 1.00 | 0.99 | 1SG | 1682 |
| ATOM | 1682 | CA | GLN | 205 | 8.245 | 6.939 | 31.833 | 1.00 | 0.99 | 1SG | 1683 |
| ATOM | 1683 | CB | GLN | 205 | 8.079 | 7.666 | 30.481 | 1.00 | 0.99 | 1SG | 1684 |
| ATOM | 1684 | CG | GLN | 205 | 6.797 | 8.501 | 30.372 | 1.00 | 0.99 | 1SG | 1685 |
| ATOM | 1685 | CD | GLN | 205 | 5.591 | 7.588 | 30.542 | 1.00 | 0.99 | 1SG | 1686 |
| ATOM | 1686 | OE1 | GLN | 205 | 5.500 | 6.531 | 29.920 | 1.00 | 0.99 | 1SG | 1687 |
| ATOM | 1687 | NE2 | GLN | 205 | 4.644 | 7.997 | 31.429 | 1.00 | 0.99 | 1SG | 1688 |
| ATOM | 1688 | C | GLN | 205 | 7.438 | 5.691 | 31.830 | 1.00 | 0.99 | 1SG | 1689 |
| ATOM | 1689 | O | GLN | 205 | 6.759 | 5.356 | 32.798 | 1.00 | 0.99 | 1SG | 1690 |
| ATOM | 1690 | N | MET | 206 | 7.509 | 4.951 | 30.716 | 1.00 | 0.80 | 1SG | 1691 |
| ATOM | 1691 | CA | MET | 206 | 6.859 | 3.686 | 30.676 | 1.00 | 0.80 | 1SG | 1692 |
| ATOM | 1692 | CB | MET | 206 | 6.402 | 3.271 | 29.269 | 1.00 | 0.80 | 1SG | 1693 |
| ATOM | 1693 | CG | MET | 206 | 5.705 | 1.909 | 29.237 | 1.00 | 0.80 | 1SG | 1694 |
| ATOM | 1694 | SD | MET | 206 | 5.194 | 1.352 | 27.584 | 1.00 | 0.80 | 1SG | 1695 |
| ATOM | 1695 | CE | MET | 206 | 4.292 | −0.101 | 28.190 | 1.00 | 0.80 | 1SG | 1696 |
| ATOM | 1696 | C | MET | 206 | 7.863 | 2.673 | 31.113 | 1.00 | 0.80 | 1SG | 1697 |
| ATOM | 1697 | O | MET | 206 | 8.990 | 2.649 | 30.621 | 1.00 | 0.80 | 1SG | 1698 |
| ATOM | 1698 | N | ASN | 207 | 7.486 | 1.835 | 32.096 | 1.00 | 0.38 | 1SG | 1699 |
| ATOM | 1699 | CA | ASN | 207 | 8.347 | 0.759 | 32.473 | 1.00 | 0.38 | 1SG | 1700 |
| ATOM | 1700 | CB | ASN | 207 | 8.443 | 0.483 | 33.981 | 1.00 | 0.38 | 1SG | 1701 |
| ATOM | 1701 | CG | ASN | 207 | 9.396 | −0.695 | 34.161 | 1.00 | 0.38 | 1SG | 1702 |
| ATOM | 1702 | OD1 | ASN | 207 | 8.973 | −1.846 | 34.083 | 1.00 | 0.38 | 1SG | 1703 |
| ATOM | 1703 | ND2 | ASN | 207 | 10.706 | −0.414 | 34.390 | 1.00 | 0.38 | 1SG | 1704 |
| ATOM | 1704 | C | ASN | 207 | 7.743 | −0.443 | 31.841 | 1.00 | 0.38 | 1SG | 1705 |
| ATOM | 1705 | O | ASN | 207 | 6.528 | −0.624 | 31.858 | 1.00 | 0.38 | 1SG | 1706 |
| ATOM | 1706 | N | ALA | 208 | 8.583 | −1.294 | 31.238 | 1.00 | 0.18 | 1SG | 1707 |
| ATOM | 1707 | CA | ALA | 208 | 8.031 | −2.405 | 30.532 | 1.00 | 0.18 | 1SG | 1708 |
| ATOM | 1708 | CB | ALA | 208 | 9.096 | −3.219 | 29.782 | 1.00 | 0.18 | 1SG | 1709 |
| ATOM | 1709 | C | ALA | 208 | 7.335 | −3.349 | 31.461 | 1.00 | 0.18 | 1SG | 1710 |
| ATOM | 1710 | O | ALA | 208 | 6.186 | −3.718 | 31.227 | 1.00 | 0.18 | 1SG | 1711 |
| ATOM | 1711 | N | GLY | 209 | 8.004 | −3.755 | 32.556 | 1.00 | 0.30 | 1SG | 1712 |
| ATOM | 1712 | CA | GLY | 209 | 7.448 | −4.814 | 33.349 | 1.00 | 0.30 | 1SG | 1713 |
| ATOM | 1713 | C | GLY | 209 | 6.147 | −4.474 | 34.011 | 1.00 | 0.30 | 1SG | 1714 |
| ATOM | 1714 | O | GLY | 209 | 5.105 | −5.019 | 33.654 | 1.00 | 0.30 | 1SG | 1715 |
| ATOM | 1715 | N | TRP | 210 | 6.167 | −3.557 | 34.995 | 1.00 | 0.53 | 1SG | 1716 |
| ATOM | 1716 | CA | TRP | 210 | 4.968 | −3.322 | 35.751 | 1.00 | 0.53 | 1SG | 1717 |
| ATOM | 1717 | CB | TRP | 210 | 5.255 | −2.721 | 37.140 | 1.00 | 0.53 | 1SG | 1718 |
| ATOM | 1718 | CG | TRP | 210 | 4.087 | −2.788 | 38.096 | 1.00 | 0.53 | 1SG | 1719 |
| ATOM | 1719 | CD2 | TRP | 210 | 3.892 | −1.905 | 39.212 | 1.00 | 0.53 | 1SG | 1720 |
| ATOM | 1720 | CD1 | TRP | 210 | 3.036 | −3.657 | 38.104 | 1.00 | 0.53 | 1SG | 1721 |
| ATOM | 1721 | NE1 | TRP | 210 | 2.204 | −3.380 | 39.163 | 1.00 | 0.53 | 1SG | 1722 |
| ATOM | 1722 | CE2 | TRP | 210 | 2.718 | −2.302 | 39.852 | 1.00 | 0.53 | 1SG | 1723 |
| ATOM | 1723 | CE3 | TRP | 210 | 4.631 | −0.851 | 39.668 | 1.00 | 0.53 | 1SG | 1724 |
| ATOM | 1724 | CZ2 | TRP | 210 | 2.263 | −1.648 | 40.961 | 1.00 | 0.53 | 1SG | 1725 |
| ATOM | 1725 | CZ3 | TRP | 210 | 4.169 | −0.193 | 40.787 | 1.00 | 0.53 | 1SG | 1726 |
| ATOM | 1726 | CH2 | TRP | 210 | 3.008 | −0.585 | 41.421 | 1.00 | 0.53 | 1SG | 1727 |
| ATOM | 1727 | C | TRP | 210 | 4.090 | −2.353 | 35.032 | 1.00 | 0.53 | 1SG | 1728 |
| ATOM | 1728 | O | TRP | 210 | 2.872 | −2.515 | 34.960 | 1.00 | 0.53 | 1SG | 1729 |
| ATOM | 1729 | N | LYS | 211 | 4.729 | −1.323 | 34.459 | 1.00 | 0.65 | 1SG | 1730 |
| ATOM | 1730 | CA | LYS | 211 | 4.091 | −0.183 | 33.877 | 1.00 | 0.65 | 1SG | 1731 |
| ATOM | 1731 | CB | LYS | 211 | 5.077 | 0.953 | 33.564 | 1.00 | 0.65 | 1SG | 1732 |
| ATOM | 1732 | CG | LYS | 211 | 5.792 | 1.490 | 34.807 | 1.00 | 0.65 | 1SG | 1733 |
| ATOM | 1733 | CD | LYS | 211 | 4.842 | 2.042 | 35.872 | 1.00 | 0.65 | 1SG | 1734 |
| ATOM | 1734 | CE | LYS | 211 | 5.557 | 2.585 | 37.111 | 1.00 | 0.65 | 1SG | 1735 |
| ATOM | 1735 | NZ | LYS | 211 | 4.564 | 3.094 | 38.084 | 1.00 | 0.65 | 1SG | 1736 |
| ATOM | 1736 | C | LYS | 211 | 3.326 | −0.504 | 32.632 | 1.00 | 0.65 | 1SG | 1737 |
| ATOM | 1737 | O | LYS | 211 | 2.417 | 0.251 | 32.289 | 1.00 | 0.65 | 1SG | 1738 |
| ATOM | 1738 | N | TYR | 212 | 3.660 | −1.604 | 31.921 | 1.00 | 0.65 | 1SG | 1739 |
| ATOM | 1739 | CA | TYR | 212 | 3.056 | −1.863 | 30.640 | 1.00 | 0.65 | 1SG | 1740 |
| ATOM | 1740 | CB | TYR | 212 | 3.467 | −3.217 | 30.007 | 1.00 | 0.65 | 1SG | 1741 |
| ATOM | 1741 | CG | TYR | 212 | 2.702 | −4.352 | 30.609 | 1.00 | 0.65 | 1SG | 1742 |
| ATOM | 1742 | CD1 | TYR | 212 | 1.482 | −4.709 | 30.077 | 1.00 | 0.65 | 1SG | 1743 |
| ATOM | 1743 | CD2 | TYR | 212 | 3.186 | −5.063 | 31.684 | 1.00 | 0.65 | 1SG | 1744 |
| ATOM | 1744 | CE1 | TYR | 212 | 0.753 | −5.749 | 30.602 | 1.00 | 0.65 | 1SG | 1745 |
| ATOM | 1745 | CE2 | TYR | 212 | 2.462 | −6.106 | 32.215 | 1.00 | 0.65 | 1SG | 1746 |
| ATOM | 1746 | CZ | TYR | 212 | 1.245 | −6.450 | 31.675 | 1.00 | 0.65 | 1SG | 1747 |
| ATOM | 1747 | OH | TYR | 212 | 0.501 | −7.518 | 32.218 | 1.00 | 0.65 | 1SG | 1748 |
| ATOM | 1748 | C | TYR | 212 | 1.564 | −1.852 | 30.785 | 1.00 | 0.65 | 1SG | 1749 |
| ATOM | 1749 | O | TYR | 212 | 0.865 | −1.289 | 29.944 | 1.00 | 0.65 | 1SG | 1750 |
| ATOM | 1750 | N | LEU | 213 | 1.036 | −2.457 | 31.865 | 1.00 | 0.62 | 1SG | 1751 |
| ATOM | 1751 | CA | LEU | 213 | −0.386 | −2.471 | 32.050 | 1.00 | 0.62 | 1SG | 1752 |
| ATOM | 1752 | CB | LEU | 213 | −0.785 | −3.182 | 33.355 | 1.00 | 0.62 | 1SG | 1753 |
| ATOM | 1753 | CG | LEU | 213 | −2.302 | −3.216 | 33.625 | 1.00 | 0.62 | 1SG | 1754 |
| ATOM | 1754 | CD2 | LEU | 213 | −2.607 | −3.594 | 35.084 | 1.00 | 0.62 | 1SG | 1755 |
| ATOM | 1755 | CD1 | LEU | 213 | −3.035 | −4.109 | 32.611 | 1.00 | 0.62 | 1SG | 1756 |
| ATOM | 1756 | C | LEU | 213 | −0.860 | −1.059 | 32.182 | 1.00 | 0.62 | 1SG | 1757 |
| ATOM | 1757 | O | LEU | 213 | −1.744 | −0.624 | 31.446 | 1.00 | 0.62 | 1SG | 1758 |
| ATOM | 1758 | N | ILE | 214 | −0.262 | −0.288 | 33.115 | 1.00 | 0.56 | 1SG | 1759 |
| ATOM | 1759 | CA | ILE | 214 | −0.700 | 1.065 | 33.278 | 1.00 | 0.56 | 1SG | 1760 |
| ATOM | 1760 | CB | ILE | 214 | −0.625 | 1.552 | 34.701 | 1.00 | 0.56 | 1SG | 1761 |
| ATOM | 1761 | CG2 | ILE | 214 | −1.605 | 0.709 | 35.535 | 1.00 | 0.56 | 1SG | 1762 |
| ATOM | 1762 | CG1 | ILE | 214 | 0.820 | 1.521 | 35.226 | 1.00 | 0.56 | 1SG | 1763 |
| ATOM | 1763 | CD1 | ILE | 214 | 1.004 | 2.290 | 36.535 | 1.00 | 0.56 | 1SG | 1764 |
| ATOM | 1764 | C | ILE | 214 | 0.196 | 1.908 | 32.441 | 1.00 | 0.56 | 1SG | 1765 |
| ATOM | 1765 | O | ILE | 214 | 0.727 | 2.927 | 32.881 | 1.00 | 0.56 | 1SG | 1766 |
| ATOM | 1766 | N | ASN | 215 | 0.346 | 1.506 | 31.172 | 1.00 | 0.44 | 1SG | 1767 |
| ATOM | 1767 | CA | ASN | 215 | 1.203 | 2.211 | 30.278 | 1.00 | 0.44 | 1SG | 1768 |
| ATOM | 1768 | CB | ASN | 215 | 1.290 | 1.533 | 28.902 | 1.00 | 0.44 | 1SG | 1769 |
| ATOM | 1769 | CG | ASN | 215 | −0.106 | 1.493 | 28.295 | 1.00 | 0.44 | 1SG | 1770 |
| ATOM | 1770 | OD1 | ASN | 215 | −1.083 | 1.139 | 28.955 | 1.00 | 0.44 | 1SG | 1771 |
| ATOM | 1771 | ND2 | ASN | 215 | −0.203 | 1.865 | 26.991 | 1.00 | 0.44 | 1SG | 1772 |
| ATOM | 1772 | C | ASN | 215 | 0.633 | 3.572 | 30.084 | 1.00 | 0.44 | 1SG | 1773 |
| ATOM | 1773 | O | ASN | 215 | 1.367 | 4.546 | 29.923 | 1.00 | 0.44 | 1SG | 1774 |
| ATOM | 1774 | N | LEU | 216 | −0.708 | 3.672 | 30.119 | 1.00 | 0.34 | 1SG | 1775 |
| ATOM | 1775 | CA | LEU | 216 | −1.324 | 4.926 | 29.816 | 1.00 | 0.34 | 1SG | 1776 |
| ATOM | 1776 | CB | LEU | 216 | −2.726 | 4.767 | 29.216 | 1.00 | 0.34 | 1SG | 1777 |
| ATOM | 1777 | CG | LEU | 216 | −2.692 | 3.950 | 27.914 | 1.00 | 0.34 | 1SG | 1778 |
| ATOM | 1778 | CD2 | LEU | 216 | −1.559 | 4.413 | 26.982 | 1.00 | 0.34 | 1SG | 1779 |
| ATOM | 1779 | CD1 | LEU | 216 | −4.067 | 3.935 | 27.233 | 1.00 | 0.34 | 1SG | 1780 |
| ATOM | 1780 | C | LEU | 216 | −1.415 | 5.788 | 31.031 | 1.00 | 0.34 | 1SG | 1781 |
| ATOM | 1781 | O | LEU | 216 | −1.658 | 5.325 | 32.143 | 1.00 | 0.34 | 1SG | 1782 |
| ATOM | 1782 | N | CYS | 217 | −1.194 | 7.097 | 30.810 | 1.00 | 0.32 | 1SG | 1783 |
| ATOM | 1783 | CA | CYS | 217 | −1.274 | 8.112 | 31.815 | 1.00 | 0.32 | 1SG | 1784 |
| ATOM | 1784 | CB | CYS | 217 | 0.106 | 8.655 | 32.236 | 1.00 | 0.32 | 1SG | 1785 |
| ATOM | 1785 | SG | CYS | 217 | 0.027 | 9.941 | 33.522 | 1.00 | 0.32 | 1SG | 1786 |
| ATOM | 1786 | C | CYS | 217 | −2.006 | 9.224 | 31.144 | 1.00 | 0.32 | 1SG | 1787 |
| ATOM | 1787 | O | CYS | 217 | −2.252 | 9.166 | 29.941 | 1.00 | 0.32 | 1SG | 1788 |
| ATOM | 1788 | N | GLY | 218 | −2.408 | 10.261 | 31.897 | 1.00 | 0.47 | 1SG | 1789 |
| ATOM | 1789 | CA | GLY | 218 | −3.081 | 11.336 | 31.237 | 1.00 | 0.47 | 1SG | 1790 |
| ATOM | 1790 | C | GLY | 218 | −2.088 | 12.440 | 31.087 | 1.00 | 0.47 | 1SG | 1791 |
| ATOM | 1791 | O | GLY | 218 | −1.769 | 13.133 | 32.052 | 1.00 | 0.47 | 1SG | 1792 |
| ATOM | 1792 | N | MET | 219 | −1.569 | 12.639 | 29.859 | 1.00 | 0.67 | 1SG | 1793 |
| ATOM | 1793 | CA | MET | 219 | −0.621 | 13.695 | 29.683 | 1.00 | 0.67 | 1SG | 1794 |
| ATOM | 1794 | CB | MET | 219 | 0.751 | 13.265 | 29.130 | 1.00 | 0.67 | 1SG | 1795 |
| ATOM | 1795 | CG | MET | 219 | 0.755 | 12.934 | 27.637 | 1.00 | 0.67 | 1SG | 1796 |
| ATOM | 1796 | SD | MET | 219 | −0.025 | 11.352 | 27.202 | 1.00 | 0.67 | 1SG | 1797 |
| ATOM | 1797 | CE | MET | 219 | 1.443 | 10.353 | 27.584 | 1.00 | 0.67 | 1SG | 1798 |
| ATOM | 1798 | C | MET | 219 | −1.193 | 14.675 | 28.717 | 1.00 | 0.67 | 1SG | 1799 |
| ATOM | 1799 | O | MET | 219 | −1.906 | 14.316 | 27.781 | 1.00 | 0.67 | 1SG | 1800 |
| ATOM | 1800 | N | ASP | 220 | −0.902 | 15.961 | 28.972 | 1.00 | 0.46 | 1SG | 1801 |
| ATOM | 1801 | CA | ASP | 220 | −1.394 | 17.056 | 28.193 | 1.00 | 0.46 | 1SG | 1802 |
| ATOM | 1802 | CB | ASP | 220 | −1.337 | 18.393 | 28.954 | 1.00 | 0.46 | 1SG | 1803 |
| ATOM | 1803 | CG | ASP | 220 | −2.423 | 18.368 | 30.021 | 1.00 | 0.46 | 1SG | 1804 |
| ATOM | 1804 | OD1 | ASP | 220 | −3.315 | 17.482 | 29.937 | 1.00 | 0.46 | 1SG | 1805 |
| ATOM | 1805 | OD2 | ASP | 220 | −2.381 | 19.240 | 30.929 | 1.00 | 0.46 | 1SG | 1806 |
| ATOM | 1806 | C | ASP | 220 | −0.656 | 17.248 | 26.895 | 1.00 | 0.46 | 1SG | 1807 |
| ATOM | 1807 | O | ASP | 220 | −1.259 | 17.666 | 25.908 | 1.00 | 0.46 | 1SG | 1808 |
| ATOM | 1808 | N | PHE | 221 | 0.658 | 16.953 | 26.825 | 1.00 | 0.44 | 1SG | 1809 |
| ATOM | 1809 | CA | PHE | 221 | 1.347 | 17.379 | 25.632 | 1.00 | 0.44 | 1SG | 1810 |
| ATOM | 1810 | CB | PHE | 221 | 2.827 | 17.754 | 25.855 | 1.00 | 0.44 | 1SG | 1811 |
| ATOM | 1811 | CG | PHE | 221 | 3.310 | 18.425 | 24.610 | 1.00 | 0.44 | 1SG | 1812 |
| ATOM | 1812 | CD1 | PHE | 221 | 3.005 | 19.745 | 24.370 | 1.00 | 0.44 | 1SG | 1813 |
| ATOM | 1813 | CD2 | PHE | 221 | 4.030 | 17.740 | 23.660 | 1.00 | 0.44 | 1SG | 1814 |
| ATOM | 1814 | CE1 | PHE | 221 | 3.429 | 20.383 | 23.228 | 1.00 | 0.44 | 1SG | 1815 |
| ATOM | 1815 | CE2 | PHE | 221 | 4.459 | 18.375 | 22.516 | 1.00 | 0.44 | 1SG | 1816 |
| ATOM | 1816 | CZ | PHE | 221 | 4.162 | 19.696 | 22.293 | 1.00 | 0.44 | 1SG | 1817 |
| ATOM | 1817 | C | PHE | 221 | 1.264 | 16.376 | 24.523 | 1.00 | 0.44 | 1SG | 1818 |
| ATOM | 1818 | O | PHE | 221 | 1.072 | 15.174 | 24.692 | 1.00 | 0.44 | 1SG | 1819 |
| ATOM | 1819 | N | PRO | 222 | 1.293 | 16.978 | 23.365 | 1.00 | 0.83 | 1SG | 1820 |
| ATOM | 1820 | CA | PRO | 222 | 1.267 | 16.257 | 22.116 | 1.00 | 0.83 | 1SG | 1821 |
| ATOM | 1821 | CD | PRO | 222 | 0.597 | 18.255 | 23.275 | 1.00 | 0.83 | 1SG | 1822 |
| ATOM | 1822 | CB | PRO | 222 | 0.754 | 17.279 | 21.094 | 1.00 | 0.83 | 1SG | 1823 |
| ATOM | 1823 | CG | PRO | 222 | −0.076 | 18.276 | 21.905 | 1.00 | 0.83 | 1SG | 1824 |
| ATOM | 1824 | C | PRO | 222 | 2.528 | 15.562 | 21.616 | 1.00 | 0.83 | 1SG | 1825 |
| ATOM | 1825 | O | PRO | 222 | 3.359 | 16.305 | 21.099 | 1.00 | 0.83 | 1SG | 1826 |
| ATOM | 1826 | N | ILE | 223 | 2.639 | 14.183 | 21.640 | 1.00 | 0.96 | 1SG | 1827 |
| ATOM | 1827 | CA | ILE | 223 | 3.712 | 13.389 | 21.017 | 1.00 | 0.96 | 1SG | 1828 |
| ATOM | 1828 | CB | ILE | 223 | 5.065 | 13.534 | 21.671 | 1.00 | 0.96 | 1SG | 1829 |
| ATOM | 1829 | CG2 | ILE | 223 | 5.956 | 12.416 | 21.109 | 1.00 | 0.96 | 1SG | 1830 |
| ATOM | 1830 | CG1 | ILE | 223 | 5.678 | 14.935 | 21.508 | 1.00 | 0.96 | 1SG | 1831 |
| ATOM | 1831 | CD1 | ILE | 223 | 5.989 | 15.306 | 20.059 | 1.00 | 0.96 | 1SG | 1832 |
| ATOM | 1832 | C | ILE | 223 | 3.422 | 11.883 | 21.120 | 1.00 | 0.96 | 1SG | 1833 |
| ATOM | 1833 | O | ILE | 223 | 3.335 | 11.406 | 22.247 | 1.00 | 0.96 | 1SG | 1834 |
| ATOM | 1834 | N | LYS | 224 | 3.346 | 11.097 | 19.982 | 1.00 | 1.00 | 1SG | 1835 |
| ATOM | 1835 | CA | LYS | 224 | 3.044 | 9.650 | 19.888 | 1.00 | 1.00 | 1SG | 1836 |
| ATOM | 1836 | CB | LYS | 224 | 1.662 | 9.189 | 20.375 | 1.00 | 1.00 | 1SG | 1837 |
| ATOM | 1837 | CG | LYS | 224 | 1.189 | 9.909 | 21.618 | 1.00 | 1.00 | 1SG | 1838 |
| ATOM | 1838 | CD | LYS | 224 | −0.300 | 9.853 | 21.844 | 1.00 | 1.00 | 1SG | 1839 |
| ATOM | 1839 | CE | LYS | 224 | −1.146 | 9.935 | 20.582 | 1.00 | 1.00 | 1SG | 1840 |
| ATOM | 1840 | NZ | LYS | 224 | −2.525 | 10.354 | 20.911 | 1.00 | 1.00 | 1SG | 1841 |
| ATOM | 1841 | C | LYS | 224 | 3.009 | 9.235 | 18.417 | 1.00 | 1.00 | 1SG | 1842 |
| ATOM | 1842 | O | LYS | 224 | 4.059 | 9.185 | 17.779 | 1.00 | 1.00 | 1SG | 1843 |
| ATOM | 1843 | N | THR | 225 | 1.799 | 8.918 | 17.819 | 1.00 | 1.17 | 1SG | 1844 |
| ATOM | 1844 | CA | THR | 225 | 1.712 | 8.654 | 16.372 | 1.00 | 1.17 | 1SG | 1845 |
| ATOM | 1845 | CB | THR | 225 | 1.882 | 7.196 | 16.058 | 1.00 | 1.17 | 1SG | 1846 |
| ATOM | 1846 | OG1 | THR | 225 | 0.844 | 6.437 | 16.660 | 1.00 | 1.17 | 1SG | 1847 |
| ATOM | 1847 | CG2 | THR | 225 | 3.250 | 6.735 | 16.587 | 1.00 | 1.17 | 1SG | 1848 |
| ATOM | 1848 | C | THR | 225 | 0.395 | 9.103 | 15.675 | 1.00 | 1.17 | 1SG | 1849 |
| ATOM | 1849 | O | THR | 225 | −0.553 | 8.324 | 15.588 | 1.00 | 1.17 | 1SG | 1850 |
| ATOM | 1850 | N | ASN | 226 | 0.316 | 10.383 | 15.168 | 1.00 | 1.19 | 1SG | 1851 |
| ATOM | 1851 | CA | ASN | 226 | −0.553 | 11.340 | 14.434 | 1.00 | 1.19 | 1SG | 1852 |
| ATOM | 1852 | CB | ASN | 226 | −0.424 | 11.265 | 12.930 | 1.00 | 1.19 | 1SG | 1853 |
| ATOM | 1853 | CG | ASN | 226 | −1.185 | 12.429 | 12.309 | 1.00 | 1.19 | 1SG | 1854 |
| ATOM | 1854 | OD1 | ASN | 226 | −1.280 | 13.515 | 12.878 | 1.00 | 1.19 | 1SG | 1855 |
| ATOM | 1855 | ND2 | ASN | 226 | −1.749 | 12.192 | 11.094 | 1.00 | 1.19 | 1SG | 1856 |
| ATOM | 1856 | C | ASN | 226 | −2.040 | 11.611 | 14.710 | 1.00 | 1.19 | 1SG | 1857 |
| ATOM | 1857 | O | ASN | 226 | −2.909 | 10.904 | 14.212 | 1.00 | 1.19 | 1SG | 1858 |
| ATOM | 1858 | N | LEU | 227 | −2.306 | 12.731 | 15.453 | 1.00 | 1.03 | 1SG | 1859 |
| ATOM | 1859 | CA | LEU | 227 | −3.435 | 13.507 | 15.925 | 1.00 | 1.03 | 1SG | 1860 |
| ATOM | 1860 | CB | LEU | 227 | −3.814 | 13.250 | 17.388 | 1.00 | 1.03 | 1SG | 1861 |
| ATOM | 1861 | CG | LEU | 227 | −5.042 | 14.015 | 17.912 | 1.00 | 1.03 | 1SG | 1862 |
| ATOM | 1862 | CD2 | LEU | 227 | −4.939 | 15.534 | 17.709 | 1.00 | 1.03 | 1SG | 1863 |
| ATOM | 1863 | CD1 | LEU | 227 | −5.283 | 13.655 | 19.380 | 1.00 | 1.03 | 1SG | 1864 |
| ATOM | 1864 | C | LEU | 227 | −2.691 | 14.749 | 15.808 | 1.00 | 1.03 | 1SG | 1865 |
| ATOM | 1865 | O | LEU | 227 | −2.474 | 15.243 | 14.706 | 1.00 | 1.03 | 1SG | 1866 |
| ATOM | 1866 | N | GLU | 228 | −2.245 | 15.323 | 16.922 | 1.00 | 0.81 | 1SG | 1867 |
| ATOM | 1867 | CA | GLU | 228 | −1.062 | 15.877 | 16.408 | 1.00 | 0.81 | 1SG | 1868 |
| ATOM | 1868 | CB | GLU | 228 | −0.314 | 16.937 | 17.243 | 1.00 | 0.81 | 1SG | 1869 |
| ATOM | 1869 | CG | GLU | 228 | −1.087 | 18.243 | 17.419 | 1.00 | 0.81 | 1SG | 1870 |
| ATOM | 1870 | CD | GLU | 228 | −0.227 | 19.175 | 18.260 | 1.00 | 0.81 | 1SG | 1871 |
| ATOM | 1871 | OE1 | GLU | 228 | 0.891 | 19.524 | 17.798 | 1.00 | 0.81 | 1SG | 1872 |
| ATOM | 1872 | OE2 | GLU | 228 | −0.673 | 19.549 | 19.378 | 1.00 | 0.81 | 1SG | 1873 |
| ATOM | 1873 | C | GLU | 228 | −0.338 | 14.567 | 16.313 | 1.00 | 0.81 | 1SG | 1874 |
| ATOM | 1874 | O | GLU | 228 | 0.408 | 14.305 | 15.374 | 1.00 | 0.81 | 1SG | 1875 |
| ATOM | 1875 | N | ILE | 229 | −0.617 | 13.661 | 17.292 | 1.00 | 0.65 | 1SG | 1876 |
| ATOM | 1876 | CA | ILE | 229 | −0.177 | 12.316 | 17.161 | 1.00 | 0.65 | 1SG | 1877 |
| ATOM | 1877 | CB | ILE | 229 | 1.091 | 12.207 | 17.829 | 1.00 | 0.65 | 1SG | 1878 |
| ATOM | 1878 | CG2 | ILE | 229 | 0.754 | 11.681 | 19.195 | 1.00 | 0.65 | 1SG | 1879 |
| ATOM | 1879 | CG1 | ILE | 229 | 2.124 | 11.535 | 16.943 | 1.00 | 0.65 | 1SG | 1880 |
| ATOM | 1880 | CD1 | ILE | 229 | 2.360 | 12.244 | 15.628 | 1.00 | 0.65 | 1SG | 1881 |
| ATOM | 1881 | C | ILE | 229 | −1.296 | 11.449 | 17.744 | 1.00 | 0.65 | 1SG | 1882 |
| ATOM | 1882 | O | ILE | 229 | −1.928 | 11.809 | 18.731 | 1.00 | 0.65 | 1SG | 1883 |
| ATOM | 1883 | N | VAL | 230 | −1.586 | 10.292 | 17.107 | 1.00 | 0.60 | 1SG | 1884 |
| ATOM | 1884 | CA | VAL | 230 | −2.664 | 9.358 | 17.273 | 1.00 | 0.60 | 1SG | 1885 |
| ATOM | 1885 | CB | VAL | 230 | −2.564 | 8.467 | 18.472 | 1.00 | 0.60 | 1SG | 1886 |
| ATOM | 1886 | CG1 | VAL | 230 | −3.804 | 7.554 | 18.527 | 1.00 | 0.60 | 1SG | 1887 |
| ATOM | 1887 | CG2 | VAL | 230 | −1.233 | 7.701 | 18.378 | 1.00 | 0.60 | 1SG | 1888 |
| ATOM | 1888 | C | VAL | 230 | −3.884 | 10.188 | 17.359 | 1.00 | 0.60 | 1SG | 1889 |
| ATOM | 1889 | O | VAL | 230 | −4.270 | 10.640 | 18.433 | 1.00 | 0.60 | 1SG | 1890 |
| ATOM | 1890 | N | ARG | 231 | −4.512 | 10.398 | 16.190 | 1.00 | 0.82 | 1SG | 1891 |
| ATOM | 1891 | CA | ARG | 231 | −5.602 | 11.302 | 16.057 | 1.00 | 0.82 | 1SG | 1892 |
| ATOM | 1892 | CB | ARG | 231 | −6.200 | 11.369 | 14.650 | 1.00 | 0.82 | 1SG | 1893 |
| ATOM | 1893 | CG | ARG | 231 | −7.187 | 12.519 | 14.418 | 1.00 | 0.82 | 1SG | 1894 |
| ATOM | 1894 | CD | ARG | 231 | −8.475 | 12.427 | 15.238 | 1.00 | 0.82 | 1SG | 1895 |
| ATOM | 1895 | NE | ARG | 231 | −9.342 | 13.567 | 14.827 | 1.00 | 0.82 | 1SG | 1896 |
| ATOM | 1896 | CZ | ARG | 231 | −9.145 | 14.808 | 15.360 | 1.00 | 0.82 | 1SG | 1897 |
| ATOM | 1897 | NH1 | ARG | 231 | −8.142 | 15.020 | 16.261 | 1.00 | 0.82 | 1SG | 1898 |
| ATOM | 1898 | NH2 | ARG | 231 | −9.953 | 15.838 | 14.977 | 1.00 | 0.82 | 1SG | 1899 |
| ATOM | 1899 | C | ARG | 231 | −6.665 | 10.849 | 16.938 | 1.00 | 0.82 | 1SG | 1900 |
| ATOM | 1900 | O | ARG | 231 | −7.309 | 9.828 | 16.688 | 1.00 | 0.82 | 1SG | 1901 |
| ATOM | 1901 | N | LYS | 232 | −6.860 | 11.635 | 17.998 | 1.00 | 1.10 | 1SG | 1902 |
| ATOM | 1902 | CA | LYS | 232 | −7.865 | 11.240 | 18.896 | 1.00 | 1.10 | 1SG | 1903 |
| ATOM | 1903 | CB | LYS | 232 | −7.301 | 10.506 | 20.108 | 1.00 | 1.10 | 1SG | 1904 |
| ATOM | 1904 | CG | LYS | 232 | −6.287 | 9.403 | 19.807 | 1.00 | 1.10 | 1SG | 1905 |
| ATOM | 1905 | CD | LYS | 232 | −6.842 | 8.194 | 19.062 | 1.00 | 1.10 | 1SG | 1906 |
| ATOM | 1906 | CE | LYS | 232 | −8.038 | 7.569 | 19.779 | 1.00 | 1.10 | 1SG | 1907 |
| ATOM | 1907 | NZ | LYS | 232 | −8.002 | 7.923 | 21.219 | 1.00 | 1.10 | 1SG | 1908 |
| ATOM | 1908 | C | LYS | 232 | −8.481 | 12.482 | 19.422 | 1.00 | 1.10 | 1SG | 1909 |
| ATOM | 1909 | O | LYS | 232 | −8.001 | 13.014 | 20.413 | 1.00 | 1.10 | 1SG | 1910 |
| ATOM | 1910 | N | LEU | 233 | −9.530 | 13.008 | 18.755 | 1.00 | 1.05 | 1SG | 1911 |
| ATOM | 1911 | CA | LEU | 233 | −10.307 | 14.025 | 19.399 | 1.00 | 1.05 | 1SG | 1912 |
| ATOM | 1912 | CB | LEU | 233 | −11.379 | 14.655 | 18.494 | 1.00 | 1.05 | 1SG | 1913 |
| ATOM | 1913 | CG | LEU | 233 | −12.198 | 15.753 | 19.197 | 1.00 | 1.05 | 1SG | 1914 |
| ATOM | 1914 | CD2 | LEU | 233 | −13.365 | 16.227 | 18.315 | 1.00 | 1.05 | 1SG | 1915 |
| ATOM | 1915 | CD1 | LEU | 233 | −11.296 | 16.920 | 19.634 | 1.00 | 1.05 | 1SG | 1916 |
| ATOM | 1916 | C | LEU | 233 | −10.968 | 13.167 | 20.417 | 1.00 | 1.05 | 1SG | 1917 |
| ATOM | 1917 | O | LEU | 233 | −11.272 | 13.559 | 21.543 | 1.00 | 1.05 | 1SG | 1918 |
| ATOM | 1918 | N | LYS | 234 | −11.169 | 11.922 | 19.949 | 1.00 | 0.87 | 1SG | 1919 |
| ATOM | 1919 | CA | LYS | 234 | −11.534 | 10.698 | 20.590 | 1.00 | 0.87 | 1SG | 1920 |
| ATOM | 1920 | CB | LYS | 234 | −12.987 | 10.657 | 21.092 | 1.00 | 0.87 | 1SG | 1921 |
| ATOM | 1921 | CG | LYS | 234 | −13.208 | 11.509 | 22.348 | 1.00 | 0.87 | 1SG | 1922 |
| ATOM | 1922 | CD | LYS | 234 | −14.679 | 11.774 | 22.676 | 1.00 | 0.87 | 1SG | 1923 |
| ATOM | 1923 | CE | LYS | 234 | −15.317 | 12.902 | 21.859 | 1.00 | 0.87 | 1SG | 1924 |
| ATOM | 1924 | NZ | LYS | 234 | −16.736 | 13.059 | 22.254 | 1.00 | 0.87 | 1SG | 1925 |
| ATOM | 1925 | C | LYS | 234 | −11.391 | 9.761 | 19.437 | 1.00 | 0.87 | 1SG | 1926 |
| ATOM | 1926 | O | LYS | 234 | −12.400 | 9.390 | 18.838 | 1.00 | 0.87 | 1SG | 1927 |
| ATOM | 1927 | N | LEU | 235 | −10.130 | 9.388 | 19.084 | 1.00 | 0.86 | 1SG | 1928 |
| ATOM | 1928 | CA | LEU | 235 | −9.932 | 8.712 | 17.840 | 1.00 | 0.86 | 1SG | 1929 |
| ATOM | 1929 | CB | LEU | 235 | −10.869 | 7.514 | 17.589 | 1.00 | 0.86 | 1SG | 1930 |
| ATOM | 1930 | CG | LEU | 235 | −10.767 | 6.386 | 18.628 | 1.00 | 0.86 | 1SG | 1931 |
| ATOM | 1931 | CD2 | LEU | 235 | −11.663 | 5.205 | 18.221 | 1.00 | 0.86 | 1SG | 1932 |
| ATOM | 1932 | CD1 | LEU | 235 | −11.079 | 6.890 | 20.046 | 1.00 | 0.86 | 1SG | 1933 |
| ATOM | 1933 | C | LEU | 235 | −10.385 | 9.833 | 17.001 | 1.00 | 0.86 | 1SG | 1934 |
| ATOM | 1934 | O | LEU | 235 | −9.968 | 10.969 | 17.192 | 1.00 | 0.86 | 1SG | 1935 |
| ATOM | 1935 | N | LEU | 236 | −11.275 | 9.599 | 16.050 | 1.00 | 0.75 | 1SG | 1936 |
| ATOM | 1936 | CA | LEU | 236 | −11.820 | 10.811 | 15.544 | 1.00 | 0.75 | 1SG | 1937 |
| ATOM | 1937 | CB | LEU | 236 | −11.540 | 11.040 | 14.049 | 1.00 | 0.75 | 1SG | 1938 |
| ATOM | 1938 | CG | LEU | 236 | −12.053 | 12.364 | 13.425 | 1.00 | 0.75 | 1SG | 1939 |
| ATOM | 1939 | CD2 | LEU | 236 | −13.549 | 12.569 | 13.610 | 1.00 | 0.75 | 1SG | 1940 |
| ATOM | 1940 | CD1 | LEU | 236 | −11.699 | 12.461 | 11.932 | 1.00 | 0.75 | 1SG | 1941 |
| ATOM | 1941 | C | LEU | 236 | −13.267 | 10.577 | 15.743 | 1.00 | 0.75 | 1SG | 1942 |
| ATOM | 1942 | O | LEU | 236 | −13.831 | 9.661 | 15.153 | 1.00 | 0.75 | 1SG | 1943 |
| ATOM | 1943 | N | MET | 237 | −13.896 | 11.354 | 16.633 | 1.00 | 0.47 | 1SG | 1944 |
| ATOM | 1944 | CA | MET | 237 | −15.292 | 11.129 | 16.806 | 1.00 | 0.47 | 1SG | 1945 |
| ATOM | 1945 | CB | MET | 237 | −15.823 | 11.460 | 18.211 | 1.00 | 0.47 | 1SG | 1946 |
| ATOM | 1946 | CG | MET | 237 | −17.232 | 10.913 | 18.447 | 1.00 | 0.47 | 1SG | 1947 |
| ATOM | 1947 | SD | MET | 237 | −17.780 | 10.929 | 20.180 | 1.00 | 0.47 | 1SG | 1948 |
| ATOM | 1948 | CE | MET | 237 | −16.769 | 9.512 | 20.703 | 1.00 | 0.47 | 1SG | 1949 |
| ATOM | 1949 | C | MET | 237 | −15.967 | 12.006 | 15.806 | 1.00 | 0.47 | 1SG | 1950 |
| ATOM | 1950 | O | MET | 237 | −15.475 | 13.078 | 15.461 | 1.00 | 0.47 | 1SG | 1951 |
| ATOM | 1951 | N | GLY | 238 | −17.150 | 11.574 | 15.343 | 1.00 | 0.43 | 1SG | 1952 |
| ATOM | 1952 | CA | GLY | 238 | −17.857 | 12.235 | 14.287 | 1.00 | 0.43 | 1SG | 1953 |
| ATOM | 1953 | C | GLY | 238 | −18.093 | 13.670 | 14.650 | 1.00 | 0.43 | 1SG | 1954 |
| ATOM | 1954 | O | GLY | 238 | −18.280 | 14.498 | 13.766 | 1.00 | 0.43 | 1SG | 1955 |
| ATOM | 1955 | N | GLU | 239 | −18.119 | 14.003 | 15.953 | 1.00 | 0.72 | 1SG | 1956 |
| ATOM | 1956 | CA | GLU | 239 | −18.436 | 15.344 | 16.365 | 1.00 | 0.72 | 1SG | 1957 |
| ATOM | 1957 | CB | GLU | 239 | −18.472 | 15.525 | 17.893 | 1.00 | 0.72 | 1SG | 1958 |
| ATOM | 1958 | CG | GLU | 239 | −17.126 | 15.336 | 18.596 | 1.00 | 0.72 | 1SG | 1959 |
| ATOM | 1959 | CD | GLU | 239 | −17.199 | 16.041 | 19.947 | 1.00 | 0.72 | 1SG | 1960 |
| ATOM | 1960 | OE1 | GLU | 239 | −17.605 | 17.233 | 19.977 | 1.00 | 0.72 | 1SG | 1961 |
| ATOM | 1961 | OE2 | GLU | 239 | −16.844 | 15.406 | 20.968 | 1.00 | 0.72 | 1SG | 1962 |
| ATOM | 1962 | C | GLU | 239 | −17.443 | 16.343 | 15.824 | 1.00 | 0.72 | 1SG | 1963 |
| ATOM | 1963 | O | GLU | 239 | −17.838 | 17.427 | 15.394 | 1.00 | 0.72 | 1SG | 1964 |
| ATOM | 1964 | N | ASN | 240 | −16.139 | 15.995 | 15.827 | 1.00 | 0.88 | 1SG | 1965 |
| ATOM | 1965 | CA | ASN | 240 | −15.046 | 16.831 | 15.390 | 1.00 | 0.88 | 1SG | 1966 |
| ATOM | 1966 | CB | ASN | 240 | −14.696 | 16.723 | 13.884 | 1.00 | 0.88 | 1SG | 1967 |
| ATOM | 1967 | CG | ASN | 240 | −15.801 | 17.282 | 12.992 | 1.00 | 0.88 | 1SG | 1968 |
| ATOM | 1968 | OD1 | ASN | 240 | −15.579 | 18.283 | 12.317 | 1.00 | 0.88 | 1SG | 1969 |
| ATOM | 1969 | ND2 | ASN | 240 | −17.003 | 16.665 | 12.987 | 1.00 | 0.88 | 1SG | 1970 |
| ATOM | 1970 | C | ASN | 240 | −15.253 | 18.273 | 15.766 | 1.00 | 0.88 | 1SG | 1971 |
| ATOM | 1971 | O | ASN | 240 | −15.895 | 19.053 | 15.064 | 1.00 | 0.88 | 1SG | 1972 |
| ATOM | 1972 | N | ASN | 241 | −14.715 | 18.672 | 16.933 | 1.00 | 0.64 | 1SG | 1973 |
| ATOM | 1973 | CA | ASN | 241 | −14.798 | 20.039 | 17.362 | 1.00 | 0.64 | 1SG | 1974 |
| ATOM | 1974 | CB | ASN | 241 | −14.377 | 20.242 | 18.831 | 1.00 | 0.64 | 1SG | 1975 |
| ATOM | 1975 | CG | ASN | 241 | −14.517 | 21.721 | 19.190 | 1.00 | 0.64 | 1SG | 1976 |
| ATOM | 1976 | OD1 | ASN | 241 | −13.834 | 22.601 | 18.659 | 1.00 | 0.64 | 1SG | 1977 |
| ATOM | 1977 | ND2 | ASN | 241 | −15.445 | 22.018 | 20.137 | 1.00 | 0.64 | 1SG | 1978 |
| ATOM | 1978 | C | ASN | 241 | −13.866 | 20.875 | 16.534 | 1.00 | 0.64 | 1SG | 1979 |
| ATOM | 1979 | O | ASN | 241 | −14.176 | 22.014 | 16.190 | 1.00 | 0.64 | 1SG | 1980 |
| ATOM | 1980 | N | LEU | 242 | −12.698 | 20.309 | 16.185 | 1.00 | 0.31 | 1SG | 1981 |
| ATOM | 1981 | CA | LEU | 242 | −11.652 | 21.080 | 15.577 | 1.00 | 0.31 | 1SG | 1982 |
| ATOM | 1982 | CB | LEU | 242 | −10.393 | 20.242 | 15.302 | 1.00 | 0.31 | 1SG | 1983 |
| ATOM | 1983 | CG | LEU | 242 | −9.746 | 19.659 | 16.575 | 1.00 | 0.31 | 1SG | 1984 |
| ATOM | 1984 | CD2 | LEU | 242 | −9.490 | 20.751 | 17.627 | 1.00 | 0.31 | 1SG | 1985 |
| ATOM | 1985 | CD1 | LEU | 242 | −8.493 | 18.838 | 16.231 | 1.00 | 0.31 | 1SG | 1986 |
| ATOM | 1986 | C | LEU | 242 | −12.071 | 21.687 | 14.278 | 1.00 | 0.31 | 1SG | 1987 |
| ATOM | 1987 | O | LEU | 242 | −11.887 | 22.886 | 14.064 | 1.00 | 0.31 | 1SG | 1988 |
| ATOM | 1988 | N | GLU | 243 | −12.659 | 20.895 | 13.369 | 1.00 | 0.16 | 1SG | 1989 |
| ATOM | 1989 | CA | GLU | 243 | −12.946 | 21.420 | 12.065 | 1.00 | 0.16 | 1SG | 1990 |
| ATOM | 1990 | CB | GLU | 243 | −13.538 | 20.355 | 11.128 | 1.00 | 0.16 | 1SG | 1991 |
| ATOM | 1991 | CG | GLU | 243 | −13.857 | 20.873 | 9.726 | 1.00 | 0.16 | 1SG | 1992 |
| ATOM | 1992 | CD | GLU | 243 | −14.498 | 19.729 | 8.956 | 1.00 | 0.16 | 1SG | 1993 |
| ATOM | 1993 | OE1 | GLU | 243 | −14.168 | 18.554 | 9.266 | 1.00 | 0.16 | 1SG | 1994 |
| ATOM | 1994 | OE2 | GLU | 243 | −15.333 | 20.012 | 8.055 | 1.00 | 0.16 | 1SG | 1995 |
| ATOM | 1995 | C | GLU | 243 | −13.947 | 22.528 | 12.135 | 1.00 | 0.16 | 1SG | 1996 |
| ATOM | 1996 | O | GLU | 243 | −13.731 | 23.609 | 11.589 | 1.00 | 0.16 | 1SG | 1997 |
| ATOM | 1997 | N | THR | 244 | −15.066 | 22.288 | 12.842 | 1.00 | 0.27 | 1SG | 1998 |
| ATOM | 1998 | CA | THR | 244 | −16.164 | 23.205 | 12.800 | 1.00 | 0.27 | 1SG | 1999 |
| ATOM | 1999 | CB | THR | 244 | −17.355 | 22.680 | 13.543 | 1.00 | 0.27 | 1SG | 2000 |
| ATOM | 2000 | OG1 | THR | 244 | −17.043 | 22.517 | 14.918 | 1.00 | 0.27 | 1SG | 2001 |
| ATOM | 2001 | CG2 | THR | 244 | −17.753 | 21.327 | 12.927 | 1.00 | 0.27 | 1SG | 2002 |
| ATOM | 2002 | C | THR | 244 | −15.839 | 24.546 | 13.372 | 1.00 | 0.27 | 1SG | 2003 |
| ATOM | 2003 | O | THR | 244 | −15.950 | 25.557 | 12.681 | 1.00 | 0.27 | 1SG | 2004 |
| ATOM | 2004 | N | GLU | 245 | −15.407 | 24.606 | 14.643 | 1.00 | 0.49 | 1SG | 2005 |
| ATOM | 2005 | CA | GLU | 245 | −15.237 | 25.910 | 15.211 | 1.00 | 0.49 | 1SG | 2006 |
| ATOM | 2006 | CB | GLU | 245 | −14.992 | 25.919 | 16.728 | 1.00 | 0.49 | 1SG | 2007 |
| ATOM | 2007 | CG | GLU | 245 | −16.251 | 25.628 | 17.547 | 1.00 | 0.49 | 1SG | 2008 |
| ATOM | 2008 | CD | GLU | 245 | −15.947 | 25.978 | 18.995 | 1.00 | 0.49 | 1SG | 2009 |
| ATOM | 2009 | OE1 | GLU | 245 | −14.827 | 25.636 | 19.460 | 1.00 | 0.49 | 1SG | 2010 |
| ATOM | 2010 | OE2 | GLU | 245 | −16.823 | 26.601 | 19.652 | 1.00 | 0.49 | 1SG | 2011 |
| ATOM | 2011 | C | GLU | 245 | −14.103 | 26.622 | 14.563 | 1.00 | 0.49 | 1SG | 2012 |
| ATOM | 2012 | O | GLU | 245 | −14.247 | 27.772 | 14.149 | 1.00 | 0.49 | 1SG | 2013 |
| ATOM | 2013 | N | LYS | 246 | −12.936 | 25.967 | 14.433 | 1.00 | 0.68 | 1SG | 2014 |
| ATOM | 2014 | CA | LYS | 246 | −11.866 | 26.736 | 13.881 | 1.00 | 0.68 | 1SG | 2015 |
| ATOM | 2015 | CB | LYS | 246 | −11.156 | 27.602 | 14.934 | 1.00 | 0.68 | 1SG | 2016 |
| ATOM | 2016 | CG | LYS | 246 | −12.043 | 28.692 | 15.534 | 1.00 | 0.68 | 1SG | 2017 |
| ATOM | 2017 | CD | LYS | 246 | −11.506 | 29.252 | 16.852 | 1.00 | 0.68 | 1SG | 2018 |
| ATOM | 2018 | CE | LYS | 246 | −10.056 | 29.728 | 16.776 | 1.00 | 0.68 | 1SG | 2019 |
| ATOM | 2019 | NZ | LYS | 246 | −9.629 | 30.249 | 18.094 | 1.00 | 0.68 | 1SG | 2020 |
| ATOM | 2020 | C | LYS | 246 | −10.827 | 25.823 | 13.329 | 1.00 | 0.68 | 1SG | 2021 |
| ATOM | 2021 | O | LYS | 246 | −10.346 | 24.915 | 14.004 | 1.00 | 0.68 | 1SG | 2022 |
| ATOM | 2022 | N | MET | 247 | −10.467 | 26.051 | 12.055 | 1.00 | 0.64 | 1SG | 2023 |
| ATOM | 2023 | CA | MET | 247 | −9.393 | 25.332 | 11.448 | 1.00 | 0.64 | 1SG | 2024 |
| ATOM | 2024 | CB | MET | 247 | −9.776 | 24.672 | 10.106 | 1.00 | 0.64 | 1SG | 2025 |
| ATOM | 2025 | CG | MET | 247 | −10.155 | 25.639 | 8.980 | 1.00 | 0.64 | 1SG | 2026 |
| ATOM | 2026 | SD | MET | 247 | −8.751 | 26.411 | 8.115 | 1.00 | 0.64 | 1SG | 2027 |
| ATOM | 2027 | CE | MET | 247 | −9.775 | 27.390 | 6.979 | 1.00 | 0.64 | 1SG | 2028 |
| ATOM | 2028 | C | MET | 247 | −8.361 | 26.379 | 11.204 | 1.00 | 0.64 | 1SG | 2029 |
| ATOM | 2029 | O | MET | 247 | −8.672 | 27.485 | 10.765 | 1.00 | 0.64 | 1SG | 2030 |
| ATOM | 2030 | N | PRO | 248 | −7.144 | 26.074 | 11.536 | 1.00 | 0.35 | 1SG | 2031 |
| ATOM | 2031 | CA | PRO | 248 | −6.118 | 27.063 | 11.362 | 1.00 | 0.35 | 1SG | 2032 |
| ATOM | 2032 | CD | PRO | 248 | −6.890 | 25.236 | 12.696 | 1.00 | 0.35 | 1SG | 2033 |
| ATOM | 2033 | CB | PRO | 248 | −5.018 | 26.726 | 12.373 | 1.00 | 0.35 | 1SG | 2034 |
| ATOM | 2034 | CG | PRO | 248 | −5.372 | 25.320 | 12.889 | 1.00 | 0.35 | 1SG | 2035 |
| ATOM | 2035 | C | PRO | 248 | −5.645 | 27.163 | 9.948 | 1.00 | 0.35 | 1SG | 2036 |
| ATOM | 2036 | O | PRO | 248 | −5.853 | 26.232 | 9.172 | 1.00 | 0.35 | 1SG | 2037 |
| ATOM | 2037 | N | SER | 249 | −5.055 | 28.317 | 9.585 | 1.00 | 0.28 | 1SG | 2038 |
| ATOM | 2038 | CA | SER | 249 | −4.503 | 28.509 | 8.278 | 1.00 | 0.28 | 1SG | 2039 |
| ATOM | 2039 | CB | SER | 249 | −4.059 | 29.962 | 8.027 | 1.00 | 0.28 | 1SG | 2040 |
| ATOM | 2040 | OG | SER | 249 | −5.183 | 30.830 | 8.057 | 1.00 | 0.28 | 1SG | 2041 |
| ATOM | 2041 | C | SER | 249 | −3.291 | 27.643 | 8.167 | 1.00 | 0.28 | 1SG | 2042 |
| ATOM | 2042 | O | SER | 249 | −3.053 | 27.021 | 7.133 | 1.00 | 0.28 | 1SG | 2043 |
| ATOM | 2043 | N | HIS | 250 | −2.482 | 27.589 | 9.243 | 1.00 | 0.46 | 1SG | 2044 |
| ATOM | 2044 | CA | HIS | 250 | −1.284 | 26.805 | 9.212 | 1.00 | 0.46 | 1SG | 2045 |
| ATOM | 2045 | ND1 | HIS | 250 | 0.871 | 29.232 | 10.099 | 1.00 | 0.46 | 1SG | 2046 |
| ATOM | 2046 | CG | HIS | 250 | 0.100 | 28.911 | 9.006 | 1.00 | 0.46 | 1SG | 2047 |
| ATOM | 2047 | CB | HIS | 250 | −0.120 | 27.516 | 8.502 | 1.00 | 0.46 | 1SG | 2048 |
| ATOM | 2048 | NE2 | HIS | 250 | 0.068 | 31.148 | 9.305 | 1.00 | 0.46 | 1SG | 2049 |
| ATOM | 2049 | CD2 | HIS | 250 | −0.383 | 30.093 | 8.533 | 1.00 | 0.46 | 1SG | 2050 |
| ATOM | 2050 | CE1 | HIS | 250 | 0.818 | 30.581 | 10.233 | 1.00 | 0.46 | 1SG | 2051 |
| ATOM | 2051 | C | HIS | 250 | −0.868 | 26.521 | 10.621 | 1.00 | 0.46 | 1SG | 2052 |
| ATOM | 2052 | O | HIS | 250 | −1.506 | 26.965 | 11.574 | 1.00 | 0.46 | 1SG | 2053 |
| ATOM | 2053 | N | LYS | 251 | 0.217 | 25.736 | 10.776 | 1.00 | 0.55 | 1SG | 2054 |
| ATOM | 2054 | CA | LYS | 251 | 0.731 | 25.362 | 12.063 | 1.00 | 0.55 | 1SG | 2055 |
| ATOM | 2055 | CB | LYS | 251 | 1.389 | 26.524 | 12.830 | 1.00 | 0.55 | 1SG | 2056 |
| ATOM | 2056 | CG | LYS | 251 | 2.659 | 27.061 | 12.167 | 1.00 | 0.55 | 1SG | 2057 |
| ATOM | 2057 | CD | LYS | 251 | 3.179 | 28.351 | 12.805 | 1.00 | 0.55 | 1SG | 2058 |
| ATOM | 2058 | CE | LYS | 251 | 2.203 | 29.525 | 12.712 | 1.00 | 0.55 | 1SG | 2059 |
| ATOM | 2059 | NZ | LYS | 251 | 2.792 | 30.723 | 13.351 | 1.00 | 0.55 | 1SG | 2060 |
| ATOM | 2060 | C | LYS | 251 | −0.371 | 24.802 | 12.908 | 1.00 | 0.55 | 1SG | 2061 |
| ATOM | 2061 | O | LYS | 251 | −0.682 | 25.335 | 13.973 | 1.00 | 0.55 | 1SG | 2062 |
| ATOM | 2062 | N | LYS | 252 | −0.981 | 23.688 | 12.454 | 1.00 | 0.53 | 1SG | 2063 |
| ATOM | 2063 | CA | LYS | 252 | −2.052 | 23.086 | 13.195 | 1.00 | 0.53 | 1SG | 2064 |
| ATOM | 2064 | CB | LYS | 252 | −2.560 | 21.766 | 12.621 | 1.00 | 0.53 | 1SG | 2065 |
| ATOM | 2065 | CG | LYS | 252 | −3.706 | 21.151 | 13.435 | 1.00 | 0.53 | 1SG | 2066 |
| ATOM | 2066 | CD | LYS | 252 | −5.006 | 21.954 | 13.386 | 1.00 | 0.53 | 1SG | 2067 |
| ATOM | 2067 | CE | LYS | 252 | −6.205 | 21.247 | 14.020 | 1.00 | 0.53 | 1SG | 2068 |
| ATOM | 2068 | NZ | LYS | 252 | −7.414 | 22.095 | 13.921 | 1.00 | 0.53 | 1SG | 2069 |
| ATOM | 2069 | C | LYS | 252 | −1.553 | 22.626 | 14.516 | 1.00 | 0.53 | 1SG | 2070 |
| ATOM | 2070 | O | LYS | 252 | −2.202 | 22.836 | 15.537 | 1.00 | 0.53 | 1SG | 2071 |
| ATOM | 2071 | N | GLU | 253 | −0.368 | 21.987 | 14.515 | 1.00 | 0.37 | 1SG | 2072 |
| ATOM | 2072 | CA | GLU | 253 | 0.140 | 21.289 | 15.664 | 1.00 | 0.37 | 1SG | 2073 |
| ATOM | 2073 | CB | GLU | 253 | 1.560 | 20.763 | 15.423 | 1.00 | 0.37 | 1SG | 2074 |
| ATOM | 2074 | CG | GLU | 253 | 1.658 | 19.769 | 14.269 | 1.00 | 0.37 | 1SG | 2075 |
| ATOM | 2075 | CD | GLU | 253 | 3.136 | 19.524 | 14.005 | 1.00 | 0.37 | 1SG | 2076 |
| ATOM | 2076 | OE1 | GLU | 253 | 3.903 | 20.524 | 13.985 | 1.00 | 0.37 | 1SG | 2077 |
| ATOM | 2077 | OE2 | GLU | 253 | 3.520 | 18.338 | 13.825 | 1.00 | 0.37 | 1SG | 2078 |
| ATOM | 2078 | C | GLU | 253 | 0.249 | 22.216 | 16.826 | 1.00 | 0.37 | 1SG | 2079 |
| ATOM | 2079 | O | GLU | 253 | −0.213 | 21.899 | 17.920 | 1.00 | 0.37 | 1SG | 2080 |
| ATOM | 2080 | N | ARG | 254 | 0.869 | 23.391 | 16.617 | 1.00 | 0.24 | 1SG | 2081 |
| ATOM | 2081 | CA | ARG | 254 | 1.061 | 24.323 | 17.688 | 1.00 | 0.24 | 1SG | 2082 |
| ATOM | 2082 | CB | ARG | 254 | 1.846 | 25.576 | 17.264 | 1.00 | 0.24 | 1SG | 2083 |
| ATOM | 2083 | CG | ARG | 254 | 3.332 | 25.321 | 17.004 | 1.00 | 0.24 | 1SG | 2084 |
| ATOM | 2084 | CD | ARG | 254 | 4.098 | 26.573 | 16.574 | 1.00 | 0.24 | 1SG | 2085 |
| ATOM | 2085 | NE | ARG | 254 | 5.532 | 26.194 | 16.429 | 1.00 | 0.24 | 1SG | 2086 |
| ATOM | 2086 | CZ | ARG | 254 | 6.368 | 26.961 | 15.671 | 1.00 | 0.24 | 1SG | 2087 |
| ATOM | 2087 | NH1 | ARG | 254 | 5.885 | 28.059 | 15.020 | 1.00 | 0.24 | 1SG | 2088 |
| ATOM | 2088 | NH2 | ARG | 254 | 7.688 | 26.630 | 15.563 | 1.00 | 0.24 | 1SG | 2089 |
| ATOM | 2089 | C | ARG | 254 | −0.277 | 24.789 | 18.155 | 1.00 | 0.24 | 1SG | 2090 |
| ATOM | 2090 | O | ARG | 254 | −0.499 | 24.963 | 19.351 | 1.00 | 0.24 | 1SG | 2091 |
| ATOM | 2091 | N | TRP | 255 | −1.205 | 24.992 | 17.203 | 1.00 | 0.44 | 1SG | 2092 |
| ATOM | 2092 | CA | TRP | 255 | −2.516 | 25.508 | 17.480 | 1.00 | 0.44 | 1SG | 2093 |
| ATOM | 2093 | CB | TRP | 255 | −3.347 | 25.640 | 16.190 | 1.00 | 0.44 | 1SG | 2094 |
| ATOM | 2094 | CG | TRP | 255 | −4.724 | 26.234 | 16.358 | 1.00 | 0.44 | 1SG | 2095 |
| ATOM | 2095 | CD2 | TRP | 255 | −5.938 | 25.468 | 16.385 | 1.00 | 0.44 | 1SG | 2096 |
| ATOM | 2096 | CD1 | TRP | 255 | −5.090 | 27.543 | 16.477 | 1.00 | 0.44 | 1SG | 2097 |
| ATOM | 2097 | NE1 | TRP | 255 | −6.456 | 27.640 | 16.582 | 1.00 | 0.44 | 1SG | 2098 |
| ATOM | 2098 | CE2 | TRP | 255 | −6.991 | 26.371 | 16.525 | 1.00 | 0.44 | 1SG | 2099 |
| ATOM | 2099 | CE3 | TRP | 255 | −6.157 | 24.124 | 16.295 | 1.00 | 0.44 | 1SG | 2100 |
| ATOM | 2100 | CZ2 | TRP | 255 | −8.286 | 25.941 | 16.579 | 1.00 | 0.44 | 1SG | 2101 |
| ATOM | 2101 | CZ3 | TRP | 255 | −7.464 | 23.693 | 16.354 | 1.00 | 0.44 | 1SG | 2102 |
| ATOM | 2102 | CH2 | TRP | 255 | −8.508 | 24.585 | 16.494 | 1.00 | 0.44 | 1SG | 2103 |
| ATOM | 2103 | C | TRP | 255 | −3.225 | 24.573 | 18.413 | 1.00 | 0.44 | 1SG | 2104 |
| ATOM | 2104 | O | TRP | 255 | −3.857 | 25.004 | 19.377 | 1.00 | 0.44 | 1SG | 2105 |
| ATOM | 2105 | N | LYS | 256 | −3.126 | 23.260 | 18.149 | 1.00 | 0.69 | 1SG | 2106 |
| ATOM | 2106 | CA | LYS | 256 | −3.751 | 22.251 | 18.967 | 1.00 | 0.69 | 1SG | 2107 |
| ATOM | 2107 | CB | LYS | 256 | −3.631 | 20.828 | 18.397 | 1.00 | 0.69 | 1SG | 2108 |
| ATOM | 2108 | CG | LYS | 256 | −4.034 | 19.724 | 19.384 | 1.00 | 0.69 | 1SG | 2109 |
| ATOM | 2109 | CD | LYS | 256 | −5.530 | 19.604 | 19.680 | 1.00 | 0.69 | 1SG | 2110 |
| ATOM | 2110 | CE | LYS | 256 | −6.244 | 20.940 | 19.843 | 1.00 | 0.69 | 1SG | 2111 |
| ATOM | 2111 | NZ | LYS | 256 | −6.532 | 21.515 | 18.509 | 1.00 | 0.69 | 1SG | 2112 |
| ATOM | 2112 | C | LYS | 256 | −3.091 | 22.220 | 20.311 | 1.00 | 0.69 | 1SG | 2113 |
| ATOM | 2113 | O | LYS | 256 | −3.691 | 21.794 | 21.295 | 1.00 | 0.69 | 1SG | 2114 |
| ATOM | 2114 | N | LYS | 257 | −1.813 | 22.628 | 20.379 | 1.00 | 0.48 | 1SG | 2115 |
| ATOM | 2115 | CA | LYS | 257 | −1.078 | 22.592 | 21.606 | 1.00 | 0.48 | 1SG | 2116 |
| ATOM | 2116 | CB | LYS | 257 | 0.393 | 22.988 | 21.410 | 1.00 | 0.48 | 1SG | 2117 |
| ATOM | 2117 | CG | LYS | 257 | 1.165 | 21.914 | 20.637 | 1.00 | 0.48 | 1SG | 2118 |
| ATOM | 2118 | CD | LYS | 257 | 2.506 | 22.381 | 20.077 | 1.00 | 0.48 | 1SG | 2119 |
| ATOM | 2119 | CE | LYS | 257 | 3.334 | 21.264 | 19.450 | 1.00 | 0.48 | 1SG | 2120 |
| ATOM | 2120 | NZ | LYS | 257 | 4.612 | 21.805 | 18.939 | 1.00 | 0.48 | 1SG | 2121 |
| ATOM | 2121 | C | LYS | 257 | −1.720 | 23.503 | 22.604 | 1.00 | 0.48 | 1SG | 2122 |
| ATOM | 2122 | O | LYS | 257 | −1.635 | 23.247 | 23.803 | 1.00 | 0.48 | 1SG | 2123 |
| ATOM | 2123 | N | HIS | 258 | −2.358 | 24.600 | 22.145 | 1.00 | 0.35 | 1SG | 2124 |
| ATOM | 2124 | CA | HIS | 258 | −3.016 | 25.498 | 23.056 | 1.00 | 0.35 | 1SG | 2125 |
| ATOM | 2125 | ND1 | HIS | 258 | −3.568 | 28.461 | 20.669 | 1.00 | 0.35 | 1SG | 2126 |
| ATOM | 2126 | CG | HIS | 258 | −3.037 | 27.595 | 21.600 | 1.00 | 0.35 | 1SG | 2127 |
| ATOM | 2127 | CB | HIS | 258 | −3.850 | 26.593 | 22.365 | 1.00 | 0.35 | 1SG | 2128 |
| ATOM | 2128 | NE2 | HIS | 258 | −1.390 | 28.897 | 20.772 | 1.00 | 0.35 | 1SG | 2129 |
| ATOM | 2129 | CD2 | HIS | 258 | −1.706 | 27.875 | 21.650 | 1.00 | 0.35 | 1SG | 2130 |
| ATOM | 2130 | CE1 | HIS | 258 | −2.540 | 29.216 | 20.206 | 1.00 | 0.35 | 1SG | 2131 |
| ATOM | 2131 | C | HIS | 258 | −3.961 | 24.697 | 23.903 | 1.00 | 0.35 | 1SG | 2132 |
| ATOM | 2132 | O | HIS | 258 | −4.497 | 23.675 | 23.477 | 1.00 | 0.35 | 1SG | 2133 |
| ATOM | 2133 | N | TYR | 259 | −4.175 | 25.144 | 25.156 | 1.00 | 0.55 | 1SG | 2134 |
| ATOM | 2134 | CA | TYR | 259 | −5.019 | 24.423 | 26.061 | 1.00 | 0.55 | 1SG | 2135 |
| ATOM | 2135 | CB | TYR | 259 | −4.958 | 24.957 | 27.506 | 1.00 | 0.55 | 1SG | 2136 |
| ATOM | 2136 | CG | TYR | 259 | −5.342 | 26.398 | 27.536 | 1.00 | 0.55 | 1SG | 2137 |
| ATOM | 2137 | CD1 | TYR | 259 | −6.658 | 26.787 | 27.639 | 1.00 | 0.55 | 1SG | 2138 |
| ATOM | 2138 | CD2 | TYR | 259 | −4.368 | 27.368 | 27.467 | 1.00 | 0.55 | 1SG | 2139 |
| ATOM | 2139 | CE1 | TYR | 259 | −6.994 | 28.121 | 27.671 | 1.00 | 0.55 | 1SG | 2140 |
| ATOM | 2140 | CE2 | TYR | 259 | −4.696 | 28.701 | 27.498 | 1.00 | 0.55 | 1SG | 2141 |
| ATOM | 2141 | CZ | TYR | 259 | −6.012 | 29.080 | 27.600 | 1.00 | 0.55 | 1SG | 2142 |
| ATOM | 2142 | OH | TYR | 259 | −6.349 | 30.450 | 27.633 | 1.00 | 0.55 | 1SG | 2143 |
| ATOM | 2143 | C | TYR | 259 | −6.437 | 24.444 | 25.596 | 1.00 | 0.55 | 1SG | 2144 |
| ATOM | 2144 | O | TYR | 259 | −7.123 | 23.424 | 25.661 | 1.00 | 0.55 | 1SG | 2145 |
| ATOM | 2145 | N | GLU | 260 | −6.902 | 25.601 | 25.084 | 1.00 | 0.75 | 1SG | 2146 |
| ATOM | 2146 | CA | GLU | 260 | −8.281 | 25.744 | 24.719 | 1.00 | 0.75 | 1SG | 2147 |
| ATOM | 2147 | CB | GLU | 260 | −8.645 | 27.160 | 24.229 | 1.00 | 0.75 | 1SG | 2148 |
| ATOM | 2148 | CG | GLU | 260 | −7.821 | 27.664 | 23.046 | 1.00 | 0.75 | 1SG | 2149 |
| ATOM | 2149 | CD | GLU | 260 | −6.508 | 28.216 | 23.584 | 1.00 | 0.75 | 1SG | 2150 |
| ATOM | 2150 | OE1 | GLU | 260 | −6.285 | 28.136 | 24.822 | 1.00 | 0.75 | 1SG | 2151 |
| ATOM | 2151 | OE2 | GLU | 260 | −5.710 | 28.731 | 22.757 | 1.00 | 0.75 | 1SG | 2152 |
| ATOM | 2152 | C | GLU | 260 | −8.632 | 24.758 | 23.658 | 1.00 | 0.75 | 1SG | 2153 |
| ATOM | 2153 | O | GLU | 260 | −9.675 | 24.110 | 23.734 | 1.00 | 0.75 | 1SG | 2154 |
| ATOM | 2154 | N | VAL | 261 | −7.769 | 24.595 | 22.644 | 1.00 | 0.85 | 1SG | 2155 |
| ATOM | 2155 | CA | VAL | 261 | −8.090 | 23.627 | 21.645 | 1.00 | 0.85 | 1SG | 2156 |
| ATOM | 2156 | CB | VAL | 261 | −7.147 | 23.662 | 20.485 | 1.00 | 0.85 | 1SG | 2157 |
| ATOM | 2157 | CG1 | VAL | 261 | −7.396 | 24.963 | 19.703 | 1.00 | 0.85 | 1SG | 2158 |
| ATOM | 2158 | CG2 | VAL | 261 | −5.721 | 23.593 | 21.027 | 1.00 | 0.85 | 1SG | 2159 |
| ATOM | 2159 | C | VAL | 261 | −8.118 | 22.310 | 22.348 | 1.00 | 0.85 | 1SG | 2160 |
| ATOM | 2160 | O | VAL | 261 | −8.911 | 21.433 | 22.004 | 1.00 | 0.85 | 1SG | 2161 |
| ATOM | 2161 | N | VAL | 262 | −7.229 | 22.145 | 23.348 | 1.00 | 0.92 | 1SG | 2162 |
| ATOM | 2162 | CA | VAL | 262 | −7.281 | 21.055 | 24.285 | 1.00 | 0.92 | 1SG | 2163 |
| ATOM | 2163 | CB | VAL | 262 | −8.643 | 20.460 | 24.535 | 1.00 | 0.92 | 1SG | 2164 |
| ATOM | 2164 | CG1 | VAL | 262 | −8.500 | 19.285 | 25.516 | 1.00 | 0.92 | 1SG | 2165 |
| ATOM | 2165 | CG2 | VAL | 262 | −9.578 | 21.575 | 25.028 | 1.00 | 0.92 | 1SG | 2166 |
| ATOM | 2166 | C | VAL | 262 | −6.347 | 19.960 | 23.949 | 1.00 | 0.92 | 1SG | 2167 |
| ATOM | 2167 | O | VAL | 262 | −6.224 | 19.522 | 22.811 | 1.00 | 0.92 | 1SG | 2168 |
| ATOM | 2168 | N | ASN | 263 | −5.722 | 19.462 | 25.028 | 1.00 | 0.89 | 1SG | 2169 |
| ATOM | 2169 | CA | ASN | 263 | −4.684 | 18.485 | 24.996 | 1.00 | 0.89 | 1SG | 2170 |
| ATOM | 2170 | CB | ASN | 263 | −4.363 | 17.890 | 26.374 | 1.00 | 0.89 | 1SG | 2171 |
| ATOM | 2171 | CG | ASN | 263 | −5.601 | 17.166 | 26.879 | 1.00 | 0.89 | 1SG | 2172 |
| ATOM | 2172 | OD1 | ASN | 263 | −6.677 | 17.752 | 26.988 | 1.00 | 0.89 | 1SG | 2173 |
| ATOM | 2173 | ND2 | ASN | 263 | −5.449 | 15.851 | 27.188 | 1.00 | 0.89 | 1SG | 2174 |
| ATOM | 2174 | C | ASN | 263 | −4.991 | 17.367 | 24.065 | 1.00 | 0.89 | 1SG | 2175 |
| ATOM | 2175 | O | ASN | 263 | −6.127 | 17.147 | 23.658 | 1.00 | 0.89 | 1SG | 2176 |
| ATOM | 2176 | N | GLY | 264 | −3.930 | 16.607 | 23.735 | 1.00 | 0.67 | 1SG | 2177 |
| ATOM | 2177 | CA | GLY | 264 | −3.991 | 15.485 | 22.849 | 1.00 | 0.67 | 1SG | 2178 |
| ATOM | 2178 | C | GLY | 264 | −2.573 | 15.026 | 22.671 | 1.00 | 0.67 | 1SG | 2179 |
| ATOM | 2179 | O | GLY | 264 | −1.855 | 14.928 | 23.663 | 1.00 | 0.67 | 1SG | 2180 |
| ATOM | 2180 | N | LYS | 265 | −2.109 | 14.738 | 21.431 | 1.00 | 0.72 | 1SG | 2181 |
| ATOM | 2181 | CA | LYS | 265 | −0.742 | 14.266 | 21.272 | 1.00 | 0.72 | 1SG | 2182 |
| ATOM | 2182 | CB | LYS | 265 | −0.560 | 12.776 | 21.577 | 1.00 | 0.72 | 1SG | 2183 |
| ATOM | 2183 | CG | LYS | 265 | −0.802 | 12.462 | 23.063 | 1.00 | 0.72 | 1SG | 2184 |
| ATOM | 2184 | CD | LYS | 265 | −2.266 | 12.215 | 23.427 | 1.00 | 0.72 | 1SG | 2185 |
| ATOM | 2185 | CE | LYS | 265 | −2.514 | 12.096 | 24.930 | 1.00 | 0.72 | 1SG | 2186 |
| ATOM | 2186 | NZ | LYS | 265 | −3.968 | 12.093 | 25.203 | 1.00 | 0.72 | 1SG | 2187 |
| ATOM | 2187 | C | LYS | 265 | −0.205 | 14.702 | 19.901 | 1.00 | 0.72 | 1SG | 2188 |
| ATOM | 2188 | O | LYS | 265 | −1.037 | 15.317 | 19.249 | 1.00 | 0.72 | 1SG | 2189 |
| ATOM | 2189 | N | LEU | 266 | 1.116 | 14.465 | 19.463 | 1.00 | 0.94 | 1SG | 2190 |
| ATOM | 2190 | CA | LEU | 266 | 1.814 | 15.059 | 18.281 | 1.00 | 0.94 | 1SG | 2191 |
| ATOM | 2191 | CB | LEU | 266 | 2.266 | 16.511 | 18.595 | 1.00 | 0.94 | 1SG | 2192 |
| ATOM | 2192 | CG | LEU | 266 | 3.105 | 17.289 | 17.553 | 1.00 | 0.94 | 1SG | 2193 |
| ATOM | 2193 | CD2 | LEU | 266 | 3.538 | 18.654 | 18.107 | 1.00 | 0.94 | 1SG | 2194 |
| ATOM | 2194 | CD1 | LEU | 266 | 2.395 | 17.412 | 16.195 | 1.00 | 0.94 | 1SG | 2195 |
| ATOM | 2195 | C | LEU | 266 | 3.050 | 14.324 | 17.686 | 1.00 | 0.94 | 1SG | 2196 |
| ATOM | 2196 | O | LEU | 266 | 3.602 | 13.395 | 18.275 | 1.00 | 0.94 | 1SG | 2197 |
| ATOM | 2197 | N | THR | 267 | 3.482 | 14.740 | 16.452 | 1.00 | 0.94 | 1SG | 2198 |
| ATOM | 2198 | CA | THR | 267 | 4.510 | 14.213 | 15.570 | 1.00 | 0.94 | 1SG | 2199 |
| ATOM | 2199 | CB | THR | 267 | 4.331 | 14.608 | 14.126 | 1.00 | 0.94 | 1SG | 2200 |
| ATOM | 2200 | OG1 | THR | 267 | 4.591 | 15.996 | 13.990 | 1.00 | 0.94 | 1SG | 2201 |
| ATOM | 2201 | CG2 | THR | 267 | 2.898 | 14.333 | 13.647 | 1.00 | 0.94 | 1SG | 2202 |
| ATOM | 2202 | C | THR | 267 | 5.857 | 14.779 | 15.853 | 1.00 | 0.94 | 1SG | 2203 |
| ATOM | 2203 | O | THR | 267 | 6.054 | 15.620 | 16.727 | 1.00 | 0.94 | 1SG | 2204 |
| ATOM | 2204 | N | ASN | 268 | 6.833 | 14.233 | 15.100 | 1.00 | 0.94 | 1SG | 2205 |
| ATOM | 2205 | CA | ASN | 268 | 8.205 | 14.634 | 15.095 | 1.00 | 0.94 | 1SG | 2206 |
| ATOM | 2206 | CB | ASN | 268 | 9.128 | 13.550 | 14.507 | 1.00 | 0.94 | 1SG | 2207 |
| ATOM | 2207 | CG | ASN | 268 | 8.760 | 13.325 | 13.041 | 1.00 | 0.94 | 1SG | 2208 |
| ATOM | 2208 | OD1 | ASN | 268 | 9.444 | 13.802 | 12.136 | 1.00 | 0.94 | 1SG | 2209 |
| ATOM | 2209 | ND2 | ASN | 268 | 7.650 | 12.580 | 12.795 | 1.00 | 0.94 | 1SG | 2210 |
| ATOM | 2210 | C | ASN | 268 | 8.426 | 15.878 | 14.286 | 1.00 | 0.94 | 1SG | 2211 |
| ATOM | 2211 | O | ASN | 268 | 9.143 | 16.782 | 14.709 | 1.00 | 0.94 | 1SG | 2212 |
| ATOM | 2212 | N | MET | 269 | 7.814 | 15.985 | 13.090 | 1.00 | 0.89 | 1SG | 2213 |
| ATOM | 2213 | CA | MET | 269 | 8.249 | 17.089 | 12.290 | 1.00 | 0.89 | 1SG | 2214 |
| ATOM | 2214 | CB | MET | 269 | 9.676 | 16.829 | 11.764 | 1.00 | 0.89 | 1SG | 2215 |
| ATOM | 2215 | CG | MET | 269 | 10.416 | 18.006 | 11.129 | 1.00 | 0.89 | 1SG | 2216 |
| ATOM | 2216 | SD | MET | 269 | 11.788 | 17.485 | 10.065 | 1.00 | 0.89 | 1SG | 2217 |
| ATOM | 2217 | CE | MET | 269 | 10.739 | 16.688 | 8.814 | 1.00 | 0.89 | 1SG | 2218 |
| ATOM | 2218 | C | MET | 269 | 7.352 | 17.207 | 11.101 | 1.00 | 0.89 | 1SG | 2219 |
| ATOM | 2219 | O | MET | 269 | 6.292 | 16.590 | 11.017 | 1.00 | 0.89 | 1SG | 2220 |
| ATOM | 2220 | N | GLY | 270 | 7.792 | 18.043 | 10.145 | 1.00 | 0.70 | 1SG | 2221 |
| ATOM | 2221 | CA | GLY | 270 | 7.112 | 18.285 | 8.921 | 1.00 | 0.70 | 1SG | 2222 |
| ATOM | 2222 | C | GLY | 270 | 8.047 | 19.103 | 8.104 | 1.00 | 0.70 | 1SG | 2223 |
| ATOM | 2223 | O | GLY | 270 | 9.155 | 18.680 | 7.779 | 1.00 | 0.70 | 1SG | 2224 |
| ATOM | 2224 | N | THR | 271 | 7.592 | 20.305 | 7.734 | 1.00 | 0.65 | 1SG | 2225 |
| ATOM | 2225 | CA | THR | 271 | 8.378 | 21.204 | 6.955 | 1.00 | 0.65 | 1SG | 2226 |
| ATOM | 2226 | CB | THR | 271 | 7.606 | 22.420 | 6.549 | 1.00 | 0.65 | 1SG | 2227 |
| ATOM | 2227 | OG1 | THR | 271 | 8.357 | 23.196 | 5.628 | 1.00 | 0.65 | 1SG | 2228 |
| ATOM | 2228 | CG2 | THR | 271 | 7.289 | 23.236 | 7.811 | 1.00 | 0.65 | 1SG | 2229 |
| ATOM | 2229 | C | THR | 271 | 9.547 | 21.653 | 7.775 | 1.00 | 0.65 | 1SG | 2230 |
| ATOM | 2230 | O | THR | 271 | 10.632 | 21.878 | 7.242 | 1.00 | 0.65 | 1SG | 2231 |
| ATOM | 2231 | N | ASP | 272 | 9.357 | 21.795 | 9.102 | 1.00 | 0.68 | 1SG | 2232 |
| ATOM | 2232 | CA | ASP | 272 | 10.406 | 22.296 | 9.945 | 1.00 | 0.68 | 1SG | 2233 |
| ATOM | 2233 | CB | ASP | 272 | 9.962 | 22.626 | 11.384 | 1.00 | 0.68 | 1SG | 2234 |
| ATOM | 2234 | CG | ASP | 272 | 9.440 | 21.366 | 12.050 | 1.00 | 0.68 | 1SG | 2235 |
| ATOM | 2235 | OD1 | ASP | 272 | 9.088 | 20.407 | 11.311 | 1.00 | 0.68 | 1SG | 2236 |
| ATOM | 2236 | OD2 | ASP | 272 | 9.381 | 21.348 | 13.310 | 1.00 | 0.68 | 1SG | 2237 |
| ATOM | 2237 | C | ASP | 272 | 11.559 | 21.342 | 9.976 | 1.00 | 0.68 | 1SG | 2238 |
| ATOM | 2238 | O | ASP | 272 | 11.506 | 20.257 | 9.402 | 1.00 | 0.68 | 1SG | 2239 |
| ATOM | 2239 | N | LYS | 273 | 12.648 | 21.771 | 10.650 | 1.00 | 0.68 | 1SG | 2240 |
| ATOM | 2240 | CA | LYS | 273 | 13.905 | 21.074 | 10.694 | 1.00 | 0.68 | 1SG | 2241 |
| ATOM | 2241 | CB | LYS | 273 | 15.025 | 21.882 | 11.378 | 1.00 | 0.68 | 1SG | 2242 |
| ATOM | 2242 | CG | LYS | 273 | 15.371 | 23.203 | 10.681 | 1.00 | 0.68 | 1SG | 2243 |
| ATOM | 2243 | CD | LYS | 273 | 15.930 | 23.052 | 9.264 | 1.00 | 0.68 | 1SG | 2244 |
| ATOM | 2244 | CE | LYS | 273 | 16.211 | 24.390 | 8.573 | 1.00 | 0.68 | 1SG | 2245 |
| ATOM | 2245 | NZ | LYS | 273 | 17.321 | 25.092 | 9.255 | 1.00 | 0.68 | 1SG | 2246 |
| ATOM | 2246 | C | LYS | 273 | 13.787 | 19.764 | 11.417 | 1.00 | 0.68 | 1SG | 2247 |
| ATOM | 2247 | O | LYS | 273 | 13.091 | 19.629 | 12.422 | 1.00 | 0.68 | 1SG | 2248 |
| ATOM | 2248 | N | ILE | 274 | 14.504 | 18.765 | 10.865 | 1.00 | 0.66 | 1SG | 2249 |
| ATOM | 2249 | CA | ILE | 274 | 14.608 | 17.386 | 11.252 | 1.00 | 0.66 | 1SG | 2250 |
| ATOM | 2250 | CB | ILE | 274 | 15.248 | 16.557 | 10.177 | 1.00 | 0.66 | 1SG | 2251 |
| ATOM | 2251 | CG2 | ILE | 274 | 16.670 | 17.094 | 9.947 | 1.00 | 0.66 | 1SG | 2252 |
| ATOM | 2252 | CG1 | ILE | 274 | 15.190 | 15.063 | 10.537 | 1.00 | 0.66 | 1SG | 2253 |
| ATOM | 2253 | CD1 | ILE | 274 | 15.561 | 14.138 | 9.379 | 1.00 | 0.66 | 1SG | 2254 |
| ATOM | 2254 | C | ILE | 274 | 15.413 | 17.170 | 12.494 | 1.00 | 0.66 | 1SG | 2255 |
| ATOM | 2255 | O | ILE | 274 | 15.192 | 16.183 | 13.192 | 1.00 | 0.66 | 1SG | 2256 |
| ATOM | 2256 | N | HIS | 275 | 16.375 | 18.063 | 12.795 | 1.00 | 0.71 | 1SG | 2257 |
| ATOM | 2257 | CA | HIS | 275 | 17.330 | 17.803 | 13.838 | 1.00 | 0.71 | 1SG | 2258 |
| ATOM | 2258 | ND1 | HIS | 275 | 18.572 | 20.302 | 11.969 | 1.00 | 0.71 | 1SG | 2259 |
| ATOM | 2259 | CG | HIS | 275 | 19.055 | 19.431 | 12.919 | 1.00 | 0.71 | 1SG | 2260 |
| ATOM | 2260 | CB | HIS | 275 | 18.269 | 18.993 | 14.119 | 1.00 | 0.71 | 1SG | 2261 |
| ATOM | 2261 | NE2 | HIS | 275 | 20.632 | 19.755 | 11.338 | 1.00 | 0.71 | 1SG | 2262 |
| ATOM | 2262 | CD2 | HIS | 275 | 20.315 | 19.106 | 12.519 | 1.00 | 0.71 | 1SG | 2263 |
| ATOM | 2263 | CE1 | HIS | 275 | 19.554 | 20.461 | 11.047 | 1.00 | 0.71 | 1SG | 2264 |
| ATOM | 2264 | C | HIS | 275 | 16.666 | 17.433 | 15.130 | 1.00 | 0.71 | 1SG | 2265 |
| ATOM | 2265 | O | HIS | 275 | 17.029 | 16.407 | 15.706 | 1.00 | 0.71 | 1SG | 2266 |
| ATOM | 2266 | N | PRO | 276 | 15.715 | 18.166 | 15.636 | 1.00 | 0.65 | 1SG | 2267 |
| ATOM | 2267 | CA | PRO | 276 | 15.164 | 17.776 | 16.902 | 1.00 | 0.65 | 1SG | 2268 |
| ATOM | 2268 | CD | PRO | 276 | 15.604 | 19.598 | 15.407 | 1.00 | 0.65 | 1SG | 2269 |
| ATOM | 2269 | CB | PRO | 276 | 14.306 | 18.951 | 17.368 | 1.00 | 0.65 | 1SG | 2270 |
| ATOM | 2270 | CG | PRO | 276 | 14.964 | 20.167 | 16.686 | 1.00 | 0.65 | 1SG | 2271 |
| ATOM | 2271 | C | PRO | 276 | 14.446 | 16.472 | 16.830 | 1.00 | 0.65 | 1SG | 2272 |
| ATOM | 2272 | O | PRO | 276 | 14.557 | 15.682 | 17.767 | 1.00 | 0.65 | 1SG | 2273 |
| ATOM | 2273 | N | PRO | 277 | 13.727 | 16.231 | 15.776 | 1.00 | 0.54 | 1SG | 2274 |
| ATOM | 2274 | CA | PRO | 277 | 13.029 | 14.987 | 15.660 | 1.00 | 0.54 | 1SG | 2275 |
| ATOM | 2275 | CD | PRO | 277 | 13.049 | 17.294 | 15.053 | 1.00 | 0.54 | 1SG | 2276 |
| ATOM | 2276 | CB | PRO | 277 | 12.011 | 15.171 | 14.526 | 1.00 | 0.54 | 1SG | 2277 |
| ATOM | 2277 | CG | PRO | 277 | 12.337 | 16.550 | 13.915 | 1.00 | 0.54 | 1SG | 2278 |
| ATOM | 2278 | C | PRO | 277 | 13.970 | 13.852 | 15.492 | 1.00 | 0.54 | 1SG | 2279 |
| ATOM | 2279 | O | PRO | 277 | 13.657 | 12.743 | 15.925 | 1.00 | 0.54 | 1SG | 2280 |
| ATOM | 2280 | N | LEU | 278 | 15.121 | 14.108 | 14.854 | 1.00 | 0.63 | 1SG | 2281 |
| ATOM | 2281 | CA | LEU | 278 | 16.085 | 13.078 | 14.670 | 1.00 | 0.63 | 1SG | 2282 |
| ATOM | 2282 | CB | LEU | 278 | 17.264 | 13.552 | 13.789 | 1.00 | 0.63 | 1SG | 2283 |
| ATOM | 2283 | CG | LEU | 278 | 18.428 | 12.553 | 13.624 | 1.00 | 0.63 | 1SG | 2284 |
| ATOM | 2284 | CD2 | LEU | 278 | 17.916 | 11.180 | 13.155 | 1.00 | 0.63 | 1SG | 2285 |
| ATOM | 2285 | CD1 | LEU | 278 | 19.294 | 12.467 | 14.891 | 1.00 | 0.63 | 1SG | 2286 |
| ATOM | 2286 | C | LEU | 278 | 16.587 | 12.693 | 16.019 | 1.00 | 0.63 | 1SG | 2287 |
| ATOM | 2287 | O | LEU | 278 | 16.737 | 11.511 | 16.322 | 1.00 | 0.63 | 1SG | 2288 |
| ATOM | 2288 | N | GLU | 279 | 16.880 | 13.691 | 16.871 | 1.00 | 0.72 | 1SG | 2289 |
| ATOM | 2289 | CA | GLU | 279 | 17.437 | 13.344 | 18.142 | 1.00 | 0.72 | 1SG | 2290 |
| ATOM | 2290 | CB | GLU | 279 | 17.843 | 14.575 | 18.960 | 1.00 | 0.72 | 1SG | 2291 |
| ATOM | 2291 | CG | GLU | 279 | 18.573 | 14.214 | 20.253 | 1.00 | 0.72 | 1SG | 2292 |
| ATOM | 2292 | CD | GLU | 279 | 18.824 | 15.501 | 21.025 | 1.00 | 0.72 | 1SG | 2293 |
| ATOM | 2293 | OE1 | GLU | 279 | 18.164 | 16.523 | 20.698 | 1.00 | 0.72 | 1SG | 2294 |
| ATOM | 2294 | OE2 | GLU | 279 | 19.673 | 15.479 | 21.956 | 1.00 | 0.72 | 1SG | 2295 |
| ATOM | 2295 | C | GLU | 279 | 16.469 | 12.574 | 18.990 | 1.00 | 0.72 | 1SG | 2296 |
| ATOM | 2296 | O | GLU | 279 | 16.649 | 11.375 | 19.199 | 1.00 | 0.72 | 1SG | 2297 |
| ATOM | 2297 | N | THR | 280 | 15.418 | 13.236 | 19.533 | 1.00 | 0.68 | 1SG | 2298 |
| ATOM | 2298 | CA | THR | 280 | 14.576 | 12.471 | 20.405 | 1.00 | 0.68 | 1SG | 2299 |
| ATOM | 2299 | CB | THR | 280 | 14.489 | 13.011 | 21.824 | 1.00 | 0.68 | 1SG | 2300 |
| ATOM | 2300 | OG1 | THR | 280 | 13.640 | 12.184 | 22.606 | 1.00 | 0.68 | 1SG | 2301 |
| ATOM | 2301 | CG2 | THR | 280 | 14.040 | 14.478 | 21.873 | 1.00 | 0.68 | 1SG | 2302 |
| ATOM | 2302 | C | THR | 280 | 13.226 | 12.067 | 19.880 | 1.00 | 0.68 | 1SG | 2303 |
| ATOM | 2303 | O | THR | 280 | 12.806 | 10.938 | 20.133 | 1.00 | 0.68 | 1SG | 2304 |
| ATOM | 2304 | N | PRO | 281 | 12.504 | 12.874 | 19.152 | 1.00 | 0.71 | 1SG | 2305 |
| ATOM | 2305 | CA | PRO | 281 | 11.158 | 12.460 | 18.875 | 1.00 | 0.71 | 1SG | 2306 |
| ATOM | 2306 | CD | PRO | 281 | 12.581 | 14.320 | 19.248 | 1.00 | 0.71 | 1SG | 2307 |
| ATOM | 2307 | CB | PRO | 281 | 10.409 | 13.698 | 18.366 | 1.00 | 0.71 | 1SG | 2308 |
| ATOM | 2308 | CG | PRO | 281 | 11.479 | 14.804 | 18.298 | 1.00 | 0.71 | 1SG | 2309 |
| ATOM | 2309 | C | PRO | 281 | 10.943 | 11.225 | 18.080 | 1.00 | 0.71 | 1SG | 2310 |
| ATOM | 2310 | O | PRO | 281 | 10.025 | 10.478 | 18.408 | 1.00 | 0.71 | 1SG | 2311 |
| ATOM | 2311 | N | LEU | 282 | 11.746 | 10.976 | 17.040 | 1.00 | 0.92 | 1SG | 2312 |
| ATOM | 2312 | CA | LEU | 282 | 11.447 | 9.826 | 16.249 | 1.00 | 0.92 | 1SG | 2313 |
| ATOM | 2313 | CB | LEU | 282 | 12.258 | 9.746 | 14.942 | 1.00 | 0.92 | 1SG | 2314 |
| ATOM | 2314 | CG | LEU | 282 | 11.913 | 8.503 | 14.099 | 1.00 | 0.92 | 1SG | 2315 |
| ATOM | 2315 | CD2 | LEU | 282 | 12.885 | 8.336 | 12.925 | 1.00 | 0.92 | 1SG | 2316 |
| ATOM | 2316 | CD1 | LEU | 282 | 10.442 | 8.523 | 13.647 | 1.00 | 0.92 | 1SG | 2317 |
| ATOM | 2317 | C | LEU | 282 | 11.723 | 8.600 | 17.045 | 1.00 | 0.92 | 1SG | 2318 |
| ATOM | 2318 | O | LEU | 282 | 10.950 | 7.643 | 17.036 | 1.00 | 0.92 | 1SG | 2319 |
| ATOM | 2319 | N | PHE | 283 | 12.832 | 8.611 | 17.796 | 1.00 | 1.03 | 1SG | 2320 |
| ATOM | 2320 | CA | PHE | 283 | 13.222 | 7.397 | 18.419 | 1.00 | 1.03 | 1SG | 2321 |
| ATOM | 2321 | CB | PHE | 283 | 14.735 | 7.294 | 18.641 | 1.00 | 1.03 | 1SG | 2322 |
| ATOM | 2322 | CG | PHE | 283 | 15.285 | 7.158 | 17.267 | 1.00 | 1.03 | 1SG | 2323 |
| ATOM | 2323 | CD1 | PHE | 283 | 15.384 | 5.923 | 16.669 | 1.00 | 1.03 | 1SG | 2324 |
| ATOM | 2324 | CD2 | PHE | 283 | 15.682 | 8.267 | 16.564 | 1.00 | 1.03 | 1SG | 2325 |
| ATOM | 2325 | CE1 | PHE | 283 | 15.885 | 5.792 | 15.393 | 1.00 | 1.03 | 1SG | 2326 |
| ATOM | 2326 | CE2 | PHE | 283 | 16.185 | 8.145 | 15.289 | 1.00 | 1.03 | 1SG | 2327 |
| ATOM | 2327 | CZ | PHE | 283 | 16.294 | 6.906 | 14.705 | 1.00 | 1.03 | 1SG | 2328 |
| ATOM | 2328 | C | PHE | 283 | 12.559 | 7.285 | 19.737 | 1.00 | 1.03 | 1SG | 2329 |
| ATOM | 2329 | O | PHE | 283 | 12.374 | 8.268 | 20.448 | 1.00 | 1.03 | 1SG | 2330 |
| ATOM | 2330 | N | SER | 284 | 12.129 | 6.049 | 20.033 | 1.00 | 0.89 | 1SG | 2331 |
| ATOM | 2331 | CA | SER | 284 | 11.572 | 5.549 | 21.256 | 1.00 | 0.89 | 1SG | 2332 |
| ATOM | 2332 | CB | SER | 284 | 11.306 | 6.611 | 22.350 | 1.00 | 0.89 | 1SG | 2333 |
| ATOM | 2333 | OG | SER | 284 | 12.525 | 7.178 | 22.823 | 1.00 | 0.89 | 1SG | 2334 |
| ATOM | 2334 | C | SER | 284 | 10.320 | 4.779 | 20.911 | 1.00 | 0.89 | 1SG | 2335 |
| ATOM | 2335 | O | SER | 284 | 9.572 | 5.170 | 20.013 | 1.00 | 0.89 | 1SG | 2336 |
| ATOM | 2336 | N | GLY | 285 | 10.080 | 3.642 | 21.606 | 1.00 | 0.73 | 1SG | 2337 |
| ATOM | 2337 | CA | GLY | 285 | 8.914 | 2.831 | 21.360 | 1.00 | 0.73 | 1SG | 2338 |
| ATOM | 2338 | C | GLY | 285 | 9.068 | 1.551 | 22.135 | 1.00 | 0.73 | 1SG | 2339 |
| ATOM | 2339 | O | GLY | 285 | 10.056 | 1.381 | 22.843 | 1.00 | 0.73 | 1SG | 2340 |
| ATOM | 2340 | N | SER | 286 | 8.105 | 0.602 | 22.020 | 1.00 | 0.62 | 1SG | 2341 |
| ATOM | 2341 | CA | SER | 286 | 8.235 | −0.612 | 22.789 | 1.00 | 0.62 | 1SG | 2342 |
| ATOM | 2342 | CB | SER | 286 | 7.126 | −0.803 | 23.835 | 1.00 | 0.62 | 1SG | 2343 |
| ATOM | 2343 | OG | SER | 286 | 7.322 | −2.013 | 24.549 | 1.00 | 0.62 | 1SG | 2344 |
| ATOM | 2344 | C | SER | 286 | 8.213 | −1.802 | 21.876 | 1.00 | 0.62 | 1SG | 2345 |
| ATOM | 2345 | O | SER | 286 | 7.388 | −1.892 | 20.966 | 1.00 | 0.62 | 1SG | 2346 |
| ATOM | 2346 | N | ALA | 287 | 9.166 | −2.733 | 22.107 | 1.00 | 0.58 | 1SG | 2347 |
| ATOM | 2347 | CA | ALA | 287 | 9.352 | −3.926 | 21.328 | 1.00 | 0.58 | 1SG | 2348 |
| ATOM | 2348 | CB | ALA | 287 | 10.633 | −4.673 | 21.732 | 1.00 | 0.58 | 1SG | 2349 |
| ATOM | 2349 | C | ALA | 287 | 8.220 | −4.887 | 21.519 | 1.00 | 0.58 | 1SG | 2350 |
| ATOM | 2350 | O | ALA | 287 | 7.623 | −5.352 | 20.545 | 1.00 | 0.58 | 1SG | 2351 |
| ATOM | 2351 | N | HIS | 288 | 7.886 | −5.218 | 22.787 | 1.00 | 0.79 | 1SG | 2352 |
| ATOM | 2352 | CA | HIS | 288 | 6.805 | −6.128 | 23.038 | 1.00 | 0.79 | 1SG | 2353 |
| ATOM | 2353 | ND1 | HIS | 288 | 6.490 | −8.065 | 26.290 | 1.00 | 0.79 | 1SG | 2354 |
| ATOM | 2354 | CG | HIS | 288 | 6.928 | −7.074 | 25.437 | 1.00 | 0.79 | 1SG | 2355 |
| ATOM | 2355 | CB | HIS | 288 | 7.148 | −7.303 | 23.975 | 1.00 | 0.79 | 1SG | 2356 |
| ATOM | 2356 | NE2 | HIS | 288 | 6.805 | −6.253 | 27.537 | 1.00 | 0.79 | 1SG | 2357 |
| ATOM | 2357 | CD2 | HIS | 288 | 7.114 | −5.974 | 26.215 | 1.00 | 0.79 | 1SG | 2358 |
| ATOM | 2358 | CE1 | HIS | 288 | 6.435 | −7.521 | 27.529 | 1.00 | 0.79 | 1SG | 2359 |
| ATOM | 2359 | C | HIS | 288 | 5.750 | −5.258 | 23.610 | 1.00 | 0.79 | 1SG | 2360 |
| ATOM | 2360 | O | HIS | 288 | 6.052 | −4.211 | 24.175 | 1.00 | 0.79 | 1SG | 2361 |
| ATOM | 2361 | N | PHE | 289 | 4.468 | −5.638 | 23.521 | 1.00 | 0.90 | 1SG | 2362 |
| ATOM | 2362 | CA | PHE | 289 | 3.642 | −4.495 | 23.737 | 1.00 | 0.90 | 1SG | 2363 |
| ATOM | 2363 | CB | PHE | 289 | 3.933 | −3.485 | 22.614 | 1.00 | 0.90 | 1SG | 2364 |
| ATOM | 2364 | CG | PHE | 289 | 2.893 | −3.659 | 21.598 | 1.00 | 0.90 | 1SG | 2365 |
| ATOM | 2365 | CD1 | PHE | 289 | 2.983 | −4.834 | 20.892 | 1.00 | 0.90 | 1SG | 2366 |
| ATOM | 2366 | CD2 | PHE | 289 | 1.938 | −2.714 | 21.327 | 1.00 | 0.90 | 1SG | 2367 |
| ATOM | 2367 | CE1 | PHE | 289 | 2.100 | −5.117 | 19.896 | 1.00 | 0.90 | 1SG | 2368 |
| ATOM | 2368 | CE2 | PHE | 289 | 1.046 | −2.994 | 20.335 | 1.00 | 0.90 | 1SG | 2369 |
| ATOM | 2369 | CZ | PHE | 289 | 1.142 | −4.179 | 19.632 | 1.00 | 0.90 | 1SG | 2370 |
| ATOM | 2370 | C | PHE | 289 | 2.197 | −4.686 | 24.061 | 1.00 | 0.90 | 1SG | 2371 |
| ATOM | 2371 | O | PHE | 289 | 1.688 | −5.792 | 24.259 | 1.00 | 0.90 | 1SG | 2372 |
| ATOM | 2372 | N | VAL | 290 | 1.532 | −3.516 | 24.151 | 1.00 | 0.60 | 1SG | 2373 |
| ATOM | 2373 | CA | VAL | 290 | 0.198 | −3.205 | 24.590 | 1.00 | 0.60 | 1SG | 2374 |
| ATOM | 2374 | CB | VAL | 290 | −0.255 | −1.849 | 24.108 | 1.00 | 0.60 | 1SG | 2375 |
| ATOM | 2375 | CG1 | VAL | 290 | −1.712 | −1.598 | 24.548 | 1.00 | 0.60 | 1SG | 2376 |
| ATOM | 2376 | CG2 | VAL | 290 | 0.736 | −0.794 | 24.631 | 1.00 | 0.60 | 1SG | 2377 |
| ATOM | 2377 | C | VAL | 290 | −0.843 | −4.166 | 24.134 | 1.00 | 0.60 | 1SG | 2378 |
| ATOM | 2378 | O | VAL | 290 | −1.202 | −4.246 | 22.964 | 1.00 | 0.60 | 1SG | 2379 |
| ATOM | 2379 | N | VAL | 291 | −1.367 | −4.957 | 25.082 | 1.00 | 0.47 | 1SG | 2380 |
| ATOM | 2380 | CA | VAL | 291 | −2.490 | −5.776 | 24.768 | 1.00 | 0.47 | 1SG | 2381 |
| ATOM | 2381 | CB | VAL | 291 | −2.259 | −7.208 | 25.128 | 1.00 | 0.47 | 1SG | 2382 |
| ATOM | 2382 | CG1 | VAL | 291 | −3.505 | −8.014 | 24.747 | 1.00 | 0.47 | 1SG | 2383 |
| ATOM | 2383 | CG2 | VAL | 291 | −0.958 | −7.663 | 24.428 | 1.00 | 0.47 | 1SG | 2384 |
| ATOM | 2384 | C | VAL | 291 | −3.582 | −5.225 | 25.619 | 1.00 | 0.47 | 1SG | 2385 |
| ATOM | 2385 | O | VAL | 291 | −3.955 | −5.798 | 26.644 | 1.00 | 0.47 | 1SG | 2386 |
| ATOM | 2386 | N | SER | 292 | −4.155 | −4.081 | 25.189 | 1.00 | 0.81 | 1SG | 2387 |
| ATOM | 2387 | CA | SER | 292 | −5.092 | −3.425 | 26.052 | 1.00 | 0.81 | 1SG | 2388 |
| ATOM | 2388 | CB | SER | 292 | −5.381 | −1.952 | 25.684 | 1.00 | 0.81 | 1SG | 2389 |
| ATOM | 2389 | OG | SER | 292 | −6.149 | −1.857 | 24.495 | 1.00 | 0.81 | 1SG | 2390 |
| ATOM | 2390 | C | SER | 292 | −6.388 | −4.154 | 26.031 | 1.00 | 0.81 | 1SG | 2391 |
| ATOM | 2391 | O | SER | 292 | −7.412 | −3.587 | 26.408 | 1.00 | 0.81 | 1SG | 2392 |
| ATOM | 2392 | N | ARG | 293 | −6.385 | −5.422 | 25.574 | 1.00 | 1.17 | 1SG | 2393 |
| ATOM | 2393 | CA | ARG | 293 | −7.618 | −6.135 | 25.517 | 1.00 | 1.17 | 1SG | 2394 |
| ATOM | 2394 | CB | ARG | 293 | −7.588 | −7.418 | 24.654 | 1.00 | 1.17 | 1SG | 2395 |
| ATOM | 2395 | CG | ARG | 293 | −8.952 | −8.115 | 24.556 | 1.00 | 1.17 | 1SG | 2396 |
| ATOM | 2396 | CD | ARG | 293 | −9.240 | −9.035 | 25.744 | 1.00 | 1.17 | 1SG | 2397 |
| ATOM | 2397 | NE | ARG | 293 | −10.660 | −9.484 | 25.694 | 1.00 | 1.17 | 1SG | 2398 |
| ATOM | 2398 | CZ | ARG | 293 | −11.189 | −10.082 | 26.800 | 1.00 | 1.17 | 1SG | 2399 |
| ATOM | 2399 | NH1 | ARG | 293 | −10.406 | −10.256 | 27.905 | 1.00 | 1.17 | 1SG | 2400 |
| ATOM | 2400 | NH2 | ARG | 293 | −12.488 | −10.498 | 26.806 | 1.00 | 1.17 | 1SG | 2401 |
| ATOM | 2401 | C | ARG | 293 | −7.905 | −6.525 | 26.921 | 1.00 | 1.17 | 1SG | 2402 |
| ATOM | 2402 | O | ARG | 293 | −7.560 | −7.614 | 27.370 | 1.00 | 1.17 | 1SG | 2403 |
| ATOM | 2403 | N | GLU | 294 | −8.505 | −5.591 | 27.673 | 1.00 | 1.17 | 1SG | 2404 |
| ATOM | 2404 | CA | GLU | 294 | −8.900 | −5.803 | 29.029 | 1.00 | 1.17 | 1SG | 2405 |
| ATOM | 2405 | CB | GLU | 294 | −7.787 | −6.340 | 29.951 | 1.00 | 1.17 | 1SG | 2406 |
| ATOM | 2406 | CG | GLU | 294 | −6.527 | −5.482 | 30.057 | 1.00 | 1.17 | 1SG | 2407 |
| ATOM | 2407 | CD | GLU | 294 | −5.647 | −6.140 | 31.113 | 1.00 | 1.17 | 1SG | 2408 |
| ATOM | 2408 | OE1 | GLU | 294 | −5.887 | −5.868 | 32.322 | 1.00 | 1.17 | 1SG | 2409 |
| ATOM | 2409 | OE2 | GLU | 294 | −4.741 | −6.926 | 30.732 | 1.00 | 1.17 | 1SG | 2410 |
| ATOM | 2410 | C | GLU | 294 | −9.387 | −4.483 | 29.537 | 1.00 | 1.17 | 1SG | 2411 |
| ATOM | 2411 | O | GLU | 294 | −10.177 | −3.816 | 28.860 | 1.00 | 1.17 | 1SG | 2412 |
| ATOM | 2412 | N | TYR | 295 | −8.949 | −4.061 | 30.740 | 1.00 | 0.96 | 1SG | 2413 |
| ATOM | 2413 | CA | TYR | 295 | −9.425 | −2.809 | 31.254 | 1.00 | 0.96 | 1SG | 2414 |
| ATOM | 2414 | CB | TYR | 295 | −10.389 | −2.947 | 32.446 | 1.00 | 0.96 | 1SG | 2415 |
| ATOM | 2415 | CG | TYR | 295 | −11.613 | −3.646 | 31.967 | 1.00 | 0.96 | 1SG | 2416 |
| ATOM | 2416 | CD1 | TYR | 295 | −11.675 | −5.020 | 31.963 | 1.00 | 0.96 | 1SG | 2417 |
| ATOM | 2417 | CD2 | TYR | 295 | −12.696 | −2.925 | 31.519 | 1.00 | 0.96 | 1SG | 2418 |
| ATOM | 2418 | CE1 | TYR | 295 | −12.805 | −5.669 | 31.522 | 1.00 | 0.96 | 1SG | 2419 |
| ATOM | 2419 | CE2 | TYR | 295 | −13.827 | −3.568 | 31.077 | 1.00 | 0.96 | 1SG | 2420 |
| ATOM | 2420 | CZ | TYR | 295 | −13.884 | −4.941 | 31.080 | 1.00 | 0.96 | 1SG | 2421 |
| ATOM | 2421 | OH | TYR | 295 | −15.049 | −5.596 | 30.626 | 1.00 | 0.96 | 1SG | 2422 |
| ATOM | 2422 | C | TYR | 295 | −8.261 | −2.008 | 31.739 | 1.00 | 0.96 | 1SG | 2423 |
| ATOM | 2423 | O | TYR | 295 | −7.216 | −2.555 | 32.088 | 1.00 | 0.96 | 1SG | 2424 |
| ATOM | 2424 | N | VAL | 296 | −8.426 | −0.666 | 31.740 | 1.00 | 0.80 | 1SG | 2425 |
| ATOM | 2425 | CA | VAL | 296 | −7.410 | 0.240 | 32.192 | 1.00 | 0.80 | 1SG | 2426 |
| ATOM | 2426 | CB | VAL | 296 | −6.404 | 0.559 | 31.127 | 1.00 | 0.80 | 1SG | 2427 |
| ATOM | 2427 | CG1 | VAL | 296 | −5.642 | −0.730 | 30.770 | 1.00 | 0.80 | 1SG | 2428 |
| ATOM | 2428 | CG2 | VAL | 296 | −7.151 | 1.183 | 29.939 | 1.00 | 0.80 | 1SG | 2429 |
| ATOM | 2429 | C | VAL | 296 | −8.088 | 1.528 | 32.539 | 1.00 | 0.80 | 1SG | 2430 |
| ATOM | 2430 | O | VAL | 296 | −7.538 | 2.611 | 32.346 | 1.00 | 0.80 | 1SG | 2431 |
| ATOM | 2431 | N | GLU | 297 | −9.326 | 1.432 | 33.051 | 1.00 | 0.78 | 1SG | 2432 |
| ATOM | 2432 | CA | GLU | 297 | −10.098 | 2.584 | 33.409 | 1.00 | 0.78 | 1SG | 2433 |
| ATOM | 2433 | CB | GLU | 297 | −11.587 | 2.261 | 33.661 | 1.00 | 0.78 | 1SG | 2434 |
| ATOM | 2434 | CG | GLU | 297 | −11.844 | 1.126 | 34.661 | 1.00 | 0.78 | 1SG | 2435 |
| ATOM | 2435 | CD | GLU | 297 | −11.683 | 1.646 | 36.082 | 1.00 | 0.78 | 1SG | 2436 |
| ATOM | 2436 | OE1 | GLU | 297 | −11.944 | 2.855 | 36.311 | 1.00 | 0.78 | 1SG | 2437 |
| ATOM | 2437 | OE2 | GLU | 297 | −11.296 | 0.829 | 36.963 | 1.00 | 0.78 | 1SG | 2438 |
| ATOM | 2438 | C | GLU | 297 | −9.557 | 3.279 | 34.621 | 1.00 | 0.78 | 1SG | 2439 |
| ATOM | 2439 | O | GLU | 297 | −9.562 | 4.503 | 34.675 | 1.00 | 0.78 | 1SG | 2440 |
| ATOM | 2440 | N | TYR | 298 | −9.048 | 2.545 | 35.623 | 1.00 | 0.63 | 1SG | 2441 |
| ATOM | 2441 | CA | TYR | 298 | −8.745 | 3.166 | 36.886 | 1.00 | 0.63 | 1SG | 2442 |
| ATOM | 2442 | CB | TYR | 298 | −8.328 | 2.135 | 37.954 | 1.00 | 0.63 | 1SG | 2443 |
| ATOM | 2443 | CG | TYR | 298 | −7.204 | 1.329 | 37.396 | 1.00 | 0.63 | 1SG | 2444 |
| ATOM | 2444 | CD1 | TYR | 298 | −7.479 | 0.220 | 36.623 | 1.00 | 0.63 | 1SG | 2445 |
| ATOM | 2445 | CD2 | TYR | 298 | −5.893 | 1.687 | 37.622 | 1.00 | 0.63 | 1SG | 2446 |
| ATOM | 2446 | CE1 | TYR | 298 | −6.455 | −0.532 | 36.093 | 1.00 | 0.63 | 1SG | 2447 |
| ATOM | 2447 | CE2 | TYR | 298 | −4.872 | 0.934 | 37.093 | 1.00 | 0.63 | 1SG | 2448 |
| ATOM | 2448 | CZ | TYR | 298 | −5.148 | −0.172 | 36.327 | 1.00 | 0.63 | 1SG | 2449 |
| ATOM | 2449 | OH | TYR | 298 | −4.091 | −0.937 | 35.786 | 1.00 | 0.63 | 1SG | 2450 |
| ATOM | 2450 | C | TYR | 298 | −7.723 | 4.263 | 36.829 | 1.00 | 0.63 | 1SG | 2451 |
| ATOM | 2451 | O | TYR | 298 | −7.968 | 5.349 | 37.353 | 1.00 | 0.63 | 1SG | 2452 |
| ATOM | 2452 | N | VAL | 299 | −6.556 | 4.046 | 36.197 | 1.00 | 0.35 | 1SG | 2453 |
| ATOM | 2453 | CA | VAL | 299 | −5.562 | 5.083 | 36.243 | 1.00 | 0.35 | 1SG | 2454 |
| ATOM | 2454 | CB | VAL | 299 | −4.242 | 4.681 | 35.637 | 1.00 | 0.35 | 1SG | 2455 |
| ATOM | 2455 | CG1 | VAL | 299 | −3.329 | 5.916 | 35.596 | 1.00 | 0.35 | 1SG | 2456 |
| ATOM | 2456 | CG2 | VAL | 299 | −3.642 | 3.531 | 36.447 | 1.00 | 0.35 | 1SG | 2457 |
| ATOM | 2457 | C | VAL | 299 | −6.044 | 6.279 | 35.488 | 1.00 | 0.35 | 1SG | 2458 |
| ATOM | 2458 | O | VAL | 299 | −6.138 | 7.382 | 36.023 | 1.00 | 0.35 | 1SG | 2459 |
| ATOM | 2459 | N | LEU | 300 | −6.396 | 6.065 | 34.210 | 1.00 | 0.32 | 1SG | 2460 |
| ATOM | 2460 | CA | LEU | 300 | −6.761 | 7.093 | 33.279 | 1.00 | 0.32 | 1SG | 2461 |
| ATOM | 2461 | CB | LEU | 300 | −6.833 | 6.549 | 31.846 | 1.00 | 0.32 | 1SG | 2462 |
| ATOM | 2462 | CG | LEU | 300 | −5.470 | 5.983 | 31.379 | 1.00 | 0.32 | 1SG | 2463 |
| ATOM | 2463 | CD2 | LEU | 300 | −4.361 | 7.022 | 31.585 | 1.00 | 0.32 | 1SG | 2464 |
| ATOM | 2464 | CD1 | LEU | 300 | −5.519 | 5.444 | 29.932 | 1.00 | 0.32 | 1SG | 2465 |
| ATOM | 2465 | C | LEU | 300 | −8.062 | 7.753 | 33.623 | 1.00 | 0.32 | 1SG | 2466 |
| ATOM | 2466 | O | LEU | 300 | −8.253 | 8.920 | 33.287 | 1.00 | 0.32 | 1SG | 2467 |
| ATOM | 2467 | N | GLN | 301 | −8.996 | 7.011 | 34.257 | 1.00 | 0.46 | 1SG | 2468 |
| ATOM | 2468 | CA | GLN | 301 | −10.324 | 7.458 | 34.604 | 1.00 | 0.46 | 1SG | 2469 |
| ATOM | 2469 | CB | GLN | 301 | −11.001 | 6.593 | 35.686 | 1.00 | 0.46 | 1SG | 2470 |
| ATOM | 2470 | CG | GLN | 301 | −12.420 | 7.028 | 36.053 | 1.00 | 0.46 | 1SG | 2471 |
| ATOM | 2471 | CD | GLN | 301 | −12.922 | 6.099 | 37.153 | 1.00 | 0.46 | 1SG | 2472 |
| ATOM | 2472 | OE1 | GLN | 301 | −12.162 | 5.336 | 37.750 | 1.00 | 0.46 | 1SG | 2473 |
| ATOM | 2473 | NE2 | GLN | 301 | −14.249 | 6.170 | 37.439 | 1.00 | 0.46 | 1SG | 2474 |
| ATOM | 2474 | C | GLN | 301 | −10.251 | 8.848 | 35.135 | 1.00 | 0.46 | 1SG | 2475 |
| ATOM | 2475 | O | GLN | 301 | −9.638 | 9.115 | 36.168 | 1.00 | 0.46 | 1SG | 2476 |
| ATOM | 2476 | N | ASN | 302 | −10.912 | 9.762 | 34.407 | 1.00 | 0.58 | 1SG | 2477 |
| ATOM | 2477 | CA | ASN | 302 | −10.912 | 11.167 | 34.665 | 1.00 | 0.58 | 1SG | 2478 |
| ATOM | 2478 | CB | ASN | 302 | −11.863 | 11.879 | 33.682 | 1.00 | 0.58 | 1SG | 2479 |
| ATOM | 2479 | CG | ASN | 302 | −11.694 | 13.391 | 33.702 | 1.00 | 0.58 | 1SG | 2480 |
| ATOM | 2480 | OD1 | ASN | 302 | −11.932 | 14.029 | 32.678 | 1.00 | 0.58 | 1SG | 2481 |
| ATOM | 2481 | ND2 | ASN | 302 | −11.301 | 13.986 | 34.859 | 1.00 | 0.58 | 1SG | 2482 |
| ATOM | 2482 | C | ASN | 302 | −11.430 | 11.387 | 36.048 | 1.00 | 0.58 | 1SG | 2483 |
| ATOM | 2483 | O | ASN | 302 | −10.865 | 12.162 | 36.818 | 1.00 | 0.58 | 1SG | 2484 |
| ATOM | 2484 | N | GLN | 303 | −12.530 | 10.712 | 36.414 | 1.00 | 0.59 | 1SG | 2485 |
| ATOM | 2485 | CA | GLN | 303 | −13.059 | 10.967 | 37.718 | 1.00 | 0.59 | 1SG | 2486 |
| ATOM | 2486 | CB | GLN | 303 | −14.419 | 10.286 | 37.959 | 1.00 | 0.59 | 1SG | 2487 |
| ATOM | 2487 | CG | GLN | 303 | −14.962 | 10.488 | 39.374 | 1.00 | 0.59 | 1SG | 2488 |
| ATOM | 2488 | CD | GLN | 303 | −14.531 | 9.281 | 40.196 | 1.00 | 0.59 | 1SG | 2489 |
| ATOM | 2489 | OE1 | GLN | 303 | −14.938 | 8.159 | 39.895 | 1.00 | 0.59 | 1SG | 2490 |
| ATOM | 2490 | NE2 | GLN | 303 | −13.698 | 9.500 | 41.249 | 1.00 | 0.59 | 1SG | 2491 |
| ATOM | 2491 | C | GLN | 303 | −12.087 | 10.534 | 38.773 | 1.00 | 0.59 | 1SG | 2492 |
| ATOM | 2492 | O | GLN | 303 | −11.807 | 11.288 | 39.704 | 1.00 | 0.59 | 1SG | 2493 |
| ATOM | 2493 | N | ASN | 304 | −11.516 | 9.320 | 38.657 | 1.00 | 0.65 | 1SG | 2494 |
| ATOM | 2494 | CA | ASN | 304 | −10.640 | 8.867 | 39.700 | 1.00 | 0.65 | 1SG | 2495 |
| ATOM | 2495 | CB | ASN | 304 | −10.280 | 7.375 | 39.613 | 1.00 | 0.65 | 1SG | 2496 |
| ATOM | 2496 | CG | ASN | 304 | −9.711 | 6.975 | 40.969 | 1.00 | 0.65 | 1SG | 2497 |
| ATOM | 2497 | OD1 | ASN | 304 | −8.713 | 7.530 | 41.429 | 1.00 | 0.65 | 1SG | 2498 |
| ATOM | 2498 | ND2 | ASN | 304 | −10.367 | 5.983 | 41.629 | 1.00 | 0.65 | 1SG | 2499 |
| ATOM | 2499 | C | ASN | 304 | −9.376 | 9.669 | 39.702 | 1.00 | 0.65 | 1SG | 2500 |
| ATOM | 2500 | O | ASN | 304 | −8.839 | 9.996 | 40.760 | 1.00 | 0.65 | 1SG | 2501 |
| ATOM | 2501 | N | ILE | 305 | −8.873 | 10.033 | 38.507 | 1.00 | 0.94 | 1SG | 2502 |
| ATOM | 2502 | CA | ILE | 305 | −7.633 | 10.751 | 38.439 | 1.00 | 0.94 | 1SG | 2503 |
| ATOM | 2503 | CB | ILE | 305 | −7.196 | 11.048 | 37.027 | 1.00 | 0.94 | 1SG | 2504 |
| ATOM | 2504 | CG2 | ILE | 305 | −8.157 | 12.079 | 36.421 | 1.00 | 0.94 | 1SG | 2505 |
| ATOM | 2505 | CG1 | ILE | 305 | −5.725 | 11.498 | 36.983 | 1.00 | 0.94 | 1SG | 2506 |
| ATOM | 2506 | CD1 | ILE | 305 | −5.459 | 12.864 | 37.612 | 1.00 | 0.94 | 1SG | 2507 |
| ATOM | 2507 | C | ILE | 305 | −7.807 | 12.024 | 39.195 | 1.00 | 0.94 | 1SG | 2508 |
| ATOM | 2508 | O | ILE | 305 | −6.891 | 12.450 | 39.903 | 1.00 | 0.94 | 1SG | 2509 |
| ATOM | 2509 | N | GLN | 306 | −9.003 | 12.646 | 39.076 | 1.00 | 1.21 | 1SG | 2510 |
| ATOM | 2510 | CA | GLN | 306 | −9.295 | 13.862 | 39.778 | 1.00 | 1.21 | 1SG | 2511 |
| ATOM | 2511 | CB | GLN | 306 | −10.758 | 14.330 | 39.698 | 1.00 | 1.21 | 1SG | 2512 |
| ATOM | 2512 | CG | GLN | 306 | −10.991 | 15.624 | 40.484 | 1.00 | 1.21 | 1SG | 2513 |
| ATOM | 2513 | CD | GLN | 306 | −12.371 | 16.172 | 40.160 | 1.00 | 1.21 | 1SG | 2514 |
| ATOM | 2514 | OE1 | GLN | 306 | −12.896 | 17.036 | 40.863 | 1.00 | 1.21 | 1SG | 2515 |
| ATOM | 2515 | NE2 | GLN | 306 | −12.981 | 15.657 | 39.059 | 1.00 | 1.21 | 1SG | 2516 |
| ATOM | 2516 | C | GLN | 306 | −8.948 | 13.672 | 41.207 | 1.00 | 1.21 | 1SG | 2517 |
| ATOM | 2517 | O | GLN | 306 | −9.366 | 12.717 | 41.859 | 1.00 | 1.21 | 1SG | 2518 |
| ATOM | 2518 | N | LYS | 307 | −8.142 | 14.629 | 41.692 | 1.00 | 1.29 | 1SG | 2519 |
| ATOM | 2519 | CA | LYS | 307 | −7.494 | 14.656 | 42.960 | 1.00 | 1.29 | 1SG | 2520 |
| ATOM | 2520 | CB | LYS | 307 | −7.345 | 13.263 | 43.590 | 1.00 | 1.29 | 1SG | 2521 |
| ATOM | 2521 | CG | LYS | 307 | −6.833 | 13.219 | 45.022 | 1.00 | 1.29 | 1SG | 2522 |
| ATOM | 2522 | CD | LYS | 307 | −7.224 | 11.915 | 45.721 | 1.00 | 1.29 | 1SG | 2523 |
| ATOM | 2523 | CE | LYS | 307 | −8.679 | 11.924 | 46.197 | 1.00 | 1.29 | 1SG | 2524 |
| ATOM | 2524 | NZ | LYS | 307 | −8.836 | 12.902 | 47.295 | 1.00 | 1.29 | 1SG | 2525 |
| ATOM | 2525 | C | LYS | 307 | −6.159 | 15.077 | 42.491 | 1.00 | 1.29 | 1SG | 2526 |
| ATOM | 2526 | O | LYS | 307 | −5.946 | 16.258 | 42.216 | 1.00 | 1.29 | 1SG | 2527 |
| ATOM | 2527 | N | PHE | 308 | −5.252 | 14.093 | 42.333 | 1.00 | 1.19 | 1SG | 2528 |
| ATOM | 2528 | CA | PHE | 308 | −4.009 | 14.343 | 41.675 | 1.00 | 1.19 | 1SG | 2529 |
| ATOM | 2529 | CB | PHE | 308 | −4.187 | 15.183 | 40.400 | 1.00 | 1.19 | 1SG | 2530 |
| ATOM | 2530 | CG | PHE | 308 | −2.884 | 15.123 | 39.693 | 1.00 | 1.19 | 1SG | 2531 |
| ATOM | 2531 | CD1 | PHE | 308 | −1.867 | 16.011 | 39.968 | 1.00 | 1.19 | 1SG | 2532 |
| ATOM | 2532 | CD2 | PHE | 308 | −2.694 | 14.152 | 38.744 | 1.00 | 1.19 | 1SG | 2533 |
| ATOM | 2533 | CE1 | PHE | 308 | −0.673 | 15.914 | 39.293 | 1.00 | 1.19 | 1SG | 2534 |
| ATOM | 2534 | CE2 | PHE | 308 | −1.500 | 14.054 | 38.072 | 1.00 | 1.19 | 1SG | 2535 |
| ATOM | 2535 | CZ | PHE | 308 | −0.487 | 14.944 | 38.338 | 1.00 | 1.19 | 1SG | 2536 |
| ATOM | 2536 | C | PHE | 308 | −3.078 | 15.076 | 42.578 | 1.00 | 1.19 | 1SG | 2537 |
| ATOM | 2537 | O | PHE | 308 | −2.049 | 14.544 | 42.990 | 1.00 | 1.19 | 1SG | 2538 |
| ATOM | 2538 | N | MET | 309 | −3.443 | 16.322 | 42.910 | 1.00 | 0.95 | 1SG | 2539 |
| ATOM | 2539 | CA | MET | 309 | −2.645 | 17.221 | 43.683 | 1.00 | 0.95 | 1SG | 2540 |
| ATOM | 2540 | CB | MET | 309 | −3.302 | 18.599 | 43.869 | 1.00 | 0.95 | 1SG | 2541 |
| ATOM | 2541 | CG | MET | 309 | −2.518 | 19.502 | 44.828 | 1.00 | 0.95 | 1SG | 2542 |
| ATOM | 2542 | SD | MET | 309 | −0.861 | 20.002 | 44.250 | 1.00 | 0.95 | 1SG | 2543 |
| ATOM | 2543 | CE | MET | 309 | −1.419 | 20.891 | 42.767 | 1.00 | 0.95 | 1SG | 2544 |
| ATOM | 2544 | C | MET | 309 | −2.337 | 16.690 | 45.050 | 1.00 | 0.95 | 1SG | 2545 |
| ATOM | 2545 | O | MET | 309 | −1.223 | 16.881 | 45.536 | 1.00 | 0.95 | 1SG | 2546 |
| ATOM | 2546 | N | GLU | 310 | −3.282 | 16.016 | 45.738 | 1.00 | 0.75 | 1SG | 2547 |
| ATOM | 2547 | CA | GLU | 310 | −2.917 | 15.614 | 47.071 | 1.00 | 0.75 | 1SG | 2548 |
| ATOM | 2548 | CB | GLU | 310 | −4.054 | 15.062 | 47.955 | 1.00 | 0.75 | 1SG | 2549 |
| ATOM | 2549 | CG | GLU | 310 | −5.229 | 14.413 | 47.237 | 1.00 | 0.75 | 1SG | 2550 |
| ATOM | 2550 | CD | GLU | 310 | −6.371 | 15.392 | 47.474 | 1.00 | 0.75 | 1SG | 2551 |
| ATOM | 2551 | OE1 | GLU | 310 | −6.271 | 16.173 | 48.456 | 1.00 | 0.75 | 1SG | 2552 |
| ATOM | 2552 | OE2 | GLU | 310 | −7.359 | 15.378 | 46.694 | 1.00 | 0.75 | 1SG | 2553 |
| ATOM | 2553 | C | GLU | 310 | −1.753 | 14.674 | 47.079 | 1.00 | 0.75 | 1SG | 2554 |
| ATOM | 2554 | O | GLU | 310 | −0.829 | 14.849 | 47.872 | 1.00 | 0.75 | 1SG | 2555 |
| ATOM | 2555 | N | TRP | 311 | −1.736 | 13.656 | 46.202 | 1.00 | 0.57 | 1SG | 2556 |
| ATOM | 2556 | CA | TRP | 311 | −0.581 | 12.807 | 46.218 | 1.00 | 0.57 | 1SG | 2557 |
| ATOM | 2557 | CB | TRP | 311 | −0.680 | 11.583 | 45.303 | 1.00 | 0.57 | 1SG | 2558 |
| ATOM | 2558 | CG | TRP | 311 | −1.429 | 10.436 | 45.911 | 1.00 | 0.57 | 1SG | 2559 |
| ATOM | 2559 | CD2 | TRP | 311 | −1.384 | 9.110 | 45.374 | 1.00 | 0.57 | 1SG | 2560 |
| ATOM | 2560 | CD1 | TRP | 311 | −2.188 | 10.376 | 47.043 | 1.00 | 0.57 | 1SG | 2561 |
| ATOM | 2561 | NE1 | TRP | 311 | −2.620 | 9.087 | 47.245 | 1.00 | 0.57 | 1SG | 2562 |
| ATOM | 2562 | CE2 | TRP | 311 | −2.139 | 8.300 | 46.216 | 1.00 | 0.57 | 1SG | 2563 |
| ATOM | 2563 | CE3 | TRP | 311 | −0.769 | 8.615 | 44.259 | 1.00 | 0.57 | 1SG | 2564 |
| ATOM | 2564 | CZ2 | TRP | 311 | −2.275 | 6.966 | 45.968 | 1.00 | 0.57 | 1SG | 2565 |
| ATOM | 2565 | CZ3 | TRP | 311 | −0.917 | 7.271 | 44.003 | 1.00 | 0.57 | 1SG | 2566 |
| ATOM | 2566 | CH2 | TRP | 311 | −1.657 | 6.466 | 44.842 | 1.00 | 0.57 | 1SG | 2567 |
| ATOM | 2567 | C | TRP | 311 | 0.633 | 13.554 | 45.773 | 1.00 | 0.57 | 1SG | 2568 |
| ATOM | 2568 | O | TRP | 311 | 1.666 | 13.525 | 46.443 | 1.00 | 0.57 | 1SG | 2569 |
| ATOM | 2569 | N | ALA | 312 | 0.533 | 14.271 | 44.639 | 1.00 | 0.45 | 1SG | 2570 |
| ATOM | 2570 | CA | ALA | 312 | 1.705 | 14.890 | 44.095 | 1.00 | 0.45 | 1SG | 2571 |
| ATOM | 2571 | CB | ALA | 312 | 1.440 | 15.592 | 42.752 | 1.00 | 0.45 | 1SG | 2572 |
| ATOM | 2572 | C | ALA | 312 | 2.261 | 15.906 | 45.040 | 1.00 | 0.45 | 1SG | 2573 |
| ATOM | 2573 | O | ALA | 312 | 3.450 | 15.895 | 45.351 | 1.00 | 0.45 | 1SG | 2574 |
| ATOM | 2574 | N | LYS | 313 | 1.408 | 16.806 | 45.550 | 1.00 | 0.52 | 1SG | 2575 |
| ATOM | 2575 | CA | LYS | 313 | 1.921 | 17.840 | 46.403 | 1.00 | 0.52 | 1SG | 2576 |
| ATOM | 2576 | CB | LYS | 313 | 0.909 | 18.954 | 46.728 | 1.00 | 0.52 | 1SG | 2577 |
| ATOM | 2577 | CG | LYS | 313 | −0.153 | 18.550 | 47.749 | 1.00 | 0.52 | 1SG | 2578 |
| ATOM | 2578 | CD | LYS | 313 | −0.912 | 19.745 | 48.332 | 1.00 | 0.52 | 1SG | 2579 |
| ATOM | 2579 | CE | LYS | 313 | −0.040 | 20.680 | 49.175 | 1.00 | 0.52 | 1SG | 2580 |
| ATOM | 2580 | NZ | LYS | 313 | −0.808 | 21.887 | 49.557 | 1.00 | 0.52 | 1SG | 2581 |
| ATOM | 2581 | C | LYS | 313 | 2.392 | 17.265 | 47.707 | 1.00 | 0.52 | 1SG | 2582 |
| ATOM | 2582 | O | LYS | 313 | 3.341 | 17.778 | 48.296 | 1.00 | 0.52 | 1SG | 2583 |
| ATOM | 2583 | N | ASP | 314 | 1.735 | 16.200 | 48.210 | 1.00 | 0.59 | 1SG | 2584 |
| ATOM | 2584 | CA | ASP | 314 | 2.089 | 15.658 | 49.499 | 1.00 | 0.59 | 1SG | 2585 |
| ATOM | 2585 | CB | ASP | 314 | 1.176 | 14.516 | 49.981 | 1.00 | 0.59 | 1SG | 2586 |
| ATOM | 2586 | CG | ASP | 314 | 1.609 | 14.123 | 51.395 | 1.00 | 0.59 | 1SG | 2587 |
| ATOM | 2587 | OD1 | ASP | 314 | 2.218 | 14.984 | 52.088 | 1.00 | 0.59 | 1SG | 2588 |
| ATOM | 2588 | OD2 | ASP | 314 | 1.330 | 12.964 | 51.805 | 1.00 | 0.59 | 1SG | 2589 |
| ATOM | 2589 | C | ASP | 314 | 3.475 | 15.089 | 49.497 | 1.00 | 0.59 | 1SG | 2590 |
| ATOM | 2590 | O | ASP | 314 | 4.236 | 15.316 | 50.436 | 1.00 | 0.59 | 1SG | 2591 |
| ATOM | 2591 | N | THR | 315 | 3.849 | 14.336 | 48.445 | 1.00 | 0.69 | 1SG | 2592 |
| ATOM | 2592 | CA | THR | 315 | 5.133 | 13.696 | 48.470 | 1.00 | 0.69 | 1SG | 2593 |
| ATOM | 2593 | CB | THR | 315 | 5.424 | 12.844 | 47.270 | 1.00 | 0.69 | 1SG | 2594 |
| ATOM | 2594 | OG1 | THR | 315 | 6.688 | 12.212 | 47.421 | 1.00 | 0.69 | 1SG | 2595 |
| ATOM | 2595 | CG2 | THR | 315 | 5.403 | 13.724 | 46.010 | 1.00 | 0.69 | 1SG | 2596 |
| ATOM | 2596 | C | THR | 315 | 6.203 | 14.731 | 48.581 | 1.00 | 0.69 | 1SG | 2597 |
| ATOM | 2597 | O | THR | 315 | 6.061 | 15.849 | 48.090 | 1.00 | 0.69 | 1SG | 2598 |
| ATOM | 2598 | N | TYR | 316 | 7.315 | 14.374 | 49.258 | 1.00 | 0.76 | 1SG | 2599 |
| ATOM | 2599 | CA | TYR | 316 | 8.369 | 15.329 | 49.424 | 1.00 | 0.76 | 1SG | 2600 |
| ATOM | 2600 | CB | TYR | 316 | 9.328 | 15.036 | 50.598 | 1.00 | 0.76 | 1SG | 2601 |
| ATOM | 2601 | CG | TYR | 316 | 10.077 | 13.774 | 50.337 | 1.00 | 0.76 | 1SG | 2602 |
| ATOM | 2602 | CD1 | TYR | 316 | 9.471 | 12.553 | 50.515 | 1.00 | 0.76 | 1SG | 2603 |
| ATOM | 2603 | CD2 | TYR | 316 | 11.394 | 13.816 | 49.938 | 1.00 | 0.76 | 1SG | 2604 |
| ATOM | 2604 | CE1 | TYR | 316 | 10.164 | 11.390 | 50.278 | 1.00 | 0.76 | 1SG | 2605 |
| ATOM | 2605 | CE2 | TYR | 316 | 12.093 | 12.656 | 49.701 | 1.00 | 0.76 | 1SG | 2606 |
| ATOM | 2606 | CZ | TYR | 316 | 11.475 | 11.440 | 49.869 | 1.00 | 0.76 | 1SG | 2607 |
| ATOM | 2607 | OH | TYR | 316 | 12.188 | 10.246 | 49.626 | 1.00 | 0.76 | 1SG | 2608 |
| ATOM | 2608 | C | TYR | 316 | 9.145 | 15.383 | 48.153 | 1.00 | 0.76 | 1SG | 2609 |
| ATOM | 2609 | O | TYR | 316 | 9.020 | 14.513 | 47.292 | 1.00 | 0.76 | 1SG | 2610 |
| ATOM | 2610 | N | SER | 317 | 9.959 | 16.441 | 47.997 | 1.00 | 0.72 | 1SG | 2611 |
| ATOM | 2611 | CA | SER | 317 | 10.693 | 16.602 | 46.780 | 1.00 | 0.72 | 1SG | 2612 |
| ATOM | 2612 | CB | SER | 317 | 10.723 | 18.055 | 46.281 | 1.00 | 0.72 | 1SG | 2613 |
| ATOM | 2613 | OG | SER | 317 | 11.464 | 18.148 | 45.073 | 1.00 | 0.72 | 1SG | 2614 |
| ATOM | 2614 | C | SER | 317 | 12.105 | 16.180 | 46.998 | 1.00 | 0.72 | 1SG | 2615 |
| ATOM | 2615 | O | SER | 317 | 12.828 | 16.730 | 47.830 | 1.00 | 0.72 | 1SG | 2616 |
| ATOM | 2616 | N | PRO | 318 | 12.485 | 15.173 | 46.268 | 1.00 | 0.71 | 1SG | 2617 |
| ATOM | 2617 | CA | PRO | 318 | 13.851 | 14.742 | 46.338 | 1.00 | 0.71 | 1SG | 2618 |
| ATOM | 2618 | CD | PRO | 318 | 11.577 | 14.045 | 46.130 | 1.00 | 0.71 | 1SG | 2619 |
| ATOM | 2619 | CB | PRO | 318 | 13.863 | 13.274 | 45.928 | 1.00 | 0.71 | 1SG | 2620 |
| ATOM | 2620 | CG | PRO | 318 | 12.451 | 12.792 | 46.287 | 1.00 | 0.71 | 1SG | 2621 |
| ATOM | 2621 | C | PRO | 318 | 14.682 | 15.613 | 45.458 | 1.00 | 0.71 | 1SG | 2622 |
| ATOM | 2622 | O | PRO | 318 | 14.125 | 16.328 | 44.627 | 1.00 | 0.71 | 1SG | 2623 |
| ATOM | 2623 | N | ASP | 319 | 16.015 | 15.571 | 45.634 | 1.00 | 0.65 | 1SG | 2624 |
| ATOM | 2624 | CA | ASP | 319 | 16.908 | 16.376 | 44.854 | 1.00 | 0.65 | 1SG | 2625 |
| ATOM | 2625 | CB | ASP | 319 | 18.063 | 16.976 | 45.673 | 1.00 | 0.65 | 1SG | 2626 |
| ATOM | 2626 | CG | ASP | 319 | 17.484 | 18.045 | 46.589 | 1.00 | 0.65 | 1SG | 2627 |
| ATOM | 2627 | OD1 | ASP | 319 | 16.274 | 18.363 | 46.437 | 1.00 | 0.65 | 1SG | 2628 |
| ATOM | 2628 | OD2 | ASP | 319 | 18.243 | 18.557 | 47.455 | 1.00 | 0.65 | 1SG | 2629 |
| ATOM | 2629 | C | ASP | 319 | 17.517 | 15.514 | 43.794 | 1.00 | 0.65 | 1SG | 2630 |
| ATOM | 2630 | O | ASP | 319 | 16.853 | 14.677 | 43.185 | 1.00 | 0.65 | 1SG | 2631 |
| ATOM | 2631 | N | GLU | 320 | 18.826 | 15.729 | 43.561 | 1.00 | 0.67 | 1SG | 2632 |
| ATOM | 2632 | CA | GLU | 320 | 19.616 | 15.047 | 42.577 | 1.00 | 0.67 | 1SG | 2633 |
| ATOM | 2633 | CB | GLU | 320 | 21.081 | 15.518 | 42.553 | 1.00 | 0.67 | 1SG | 2634 |
| ATOM | 2634 | CG | GLU | 320 | 21.938 | 14.801 | 41.506 | 1.00 | 0.67 | 1SG | 2635 |
| ATOM | 2635 | CD | GLU | 320 | 21.559 | 15.321 | 40.127 | 1.00 | 0.67 | 1SG | 2636 |
| ATOM | 2636 | OE1 | GLU | 320 | 20.710 | 16.249 | 40.053 | 1.00 | 0.67 | 1SG | 2637 |
| ATOM | 2637 | OE2 | GLU | 320 | 22.117 | 14.793 | 39.128 | 1.00 | 0.67 | 1SG | 2638 |
| ATOM | 2638 | C | GLU | 320 | 19.616 | 13.594 | 42.909 | 1.00 | 0.67 | 1SG | 2639 |
| ATOM | 2639 | O | GLU | 320 | 19.856 | 12.752 | 42.044 | 1.00 | 0.67 | 1SG | 2640 |
| ATOM | 2640 | N | TYR | 321 | 19.331 | 13.270 | 44.182 | 1.00 | 0.51 | 1SG | 2641 |
| ATOM | 2641 | CA | TYR | 321 | 19.352 | 11.911 | 44.639 | 1.00 | 0.51 | 1SG | 2642 |
| ATOM | 2642 | CB | TYR | 321 | 18.822 | 11.747 | 46.073 | 1.00 | 0.51 | 1SG | 2643 |
| ATOM | 2643 | CG | TYR | 321 | 19.771 | 12.415 | 47.007 | 1.00 | 0.51 | 1SG | 2644 |
| ATOM | 2644 | CD1 | TYR | 321 | 19.686 | 13.768 | 47.249 | 1.00 | 0.51 | 1SG | 2645 |
| ATOM | 2645 | CD2 | TYR | 321 | 20.749 | 11.687 | 47.646 | 1.00 | 0.51 | 1SG | 2646 |
| ATOM | 2646 | CE1 | TYR | 321 | 20.563 | 14.383 | 48.112 | 1.00 | 0.51 | 1SG | 2647 |
| ATOM | 2647 | CE2 | TYR | 321 | 21.629 | 12.295 | 48.510 | 1.00 | 0.51 | 1SG | 2648 |
| ATOM | 2648 | CZ | TYR | 321 | 21.536 | 13.646 | 48.744 | 1.00 | 0.51 | 1SG | 2649 |
| ATOM | 2649 | OH | TYR | 321 | 22.436 | 14.275 | 49.630 | 1.00 | 0.51 | 1SG | 2650 |
| ATOM | 2650 | C | TYR | 321 | 18.469 | 11.107 | 43.744 | 1.00 | 0.51 | 1SG | 2651 |
| ATOM | 2651 | O | TYR | 321 | 18.733 | 9.931 | 43.505 | 1.00 | 0.51 | 1SG | 2652 |
| ATOM | 2652 | N | LEU | 322 | 17.395 | 11.716 | 43.210 | 1.00 | 0.19 | 1SG | 2653 |
| ATOM | 2653 | CA | LEU | 322 | 16.513 | 10.976 | 42.360 | 1.00 | 0.19 | 1SG | 2654 |
| ATOM | 2654 | CB | LEU | 322 | 15.382 | 11.844 | 41.782 | 1.00 | 0.19 | 1SG | 2655 |
| ATOM | 2655 | CG | LEU | 322 | 14.426 | 11.066 | 40.856 | 1.00 | 0.19 | 1SG | 2656 |
| ATOM | 2656 | CD2 | LEU | 322 | 13.483 | 12.024 | 40.098 | 1.00 | 0.19 | 1SG | 2657 |
| ATOM | 2657 | CD1 | LEU | 322 | 13.667 | 9.981 | 41.633 | 1.00 | 0.19 | 1SG | 2658 |
| ATOM | 2658 | C | LEU | 322 | 17.316 | 10.457 | 41.213 | 1.00 | 0.19 | 1SG | 2659 |
| ATOM | 2659 | O | LEU | 322 | 17.213 | 9.288 | 40.842 | 1.00 | 0.19 | 1SG | 2660 |
| ATOM | 2660 | N | TRP | 323 | 18.178 | 11.316 | 40.620 | 1.00 | 0.12 | 1SG | 2661 |
| ATOM | 2661 | CA | TRP | 323 | 18.988 | 10.900 | 39.515 | 1.00 | 0.12 | 1SG | 2662 |
| ATOM | 2662 | CB | TRP | 323 | 19.845 | 12.035 | 38.929 | 1.00 | 0.12 | 1SG | 2663 |
| ATOM | 2663 | CG | TRP | 323 | 20.709 | 11.601 | 37.768 | 1.00 | 0.12 | 1SG | 2664 |
| ATOM | 2664 | CD2 | TRP | 323 | 20.214 | 11.391 | 36.436 | 1.00 | 0.12 | 1SG | 2665 |
| ATOM | 2665 | CD1 | TRP | 323 | 22.038 | 11.308 | 37.737 | 1.00 | 0.12 | 1SG | 2666 |
| ATOM | 2666 | NE1 | TRP | 323 | 22.408 | 10.930 | 36.467 | 1.00 | 0.12 | 1SG | 2667 |
| ATOM | 2667 | CE2 | TRP | 323 | 21.295 | 10.975 | 35.658 | 1.00 | 0.12 | 1SG | 2668 |
| ATOM | 2668 | CE3 | TRP | 323 | 18.963 | 11.526 | 35.911 | 1.00 | 0.12 | 1SG | 2669 |
| ATOM | 2669 | CZ2 | TRP | 323 | 21.137 | 10.689 | 34.331 | 1.00 | 0.12 | 1SG | 2670 |
| ATOM | 2670 | CZ3 | TRP | 323 | 18.811 | 11.243 | 34.572 | 1.00 | 0.12 | 1SG | 2671 |
| ATOM | 2671 | CH2 | TRP | 323 | 19.875 | 10.832 | 33.798 | 1.00 | 0.12 | 1SG | 2672 |
| ATOM | 2672 | C | TRP | 323 | 19.916 | 9.829 | 39.990 | 1.00 | 0.12 | 1SG | 2673 |
| ATOM | 2673 | O | TRP | 323 | 20.175 | 8.856 | 39.281 | 1.00 | 0.12 | 1SG | 2674 |
| ATOM | 2674 | N | ALA | 324 | 20.428 | 9.984 | 41.224 | 1.00 | 0.18 | 1SG | 2675 |
| ATOM | 2675 | CA | ALA | 324 | 21.350 | 9.043 | 41.783 | 1.00 | 0.18 | 1SG | 2676 |
| ATOM | 2676 | CB | ALA | 324 | 21.832 | 9.440 | 43.188 | 1.00 | 0.18 | 1SG | 2677 |
| ATOM | 2677 | C | ALA | 324 | 20.654 | 7.728 | 41.895 | 1.00 | 0.18 | 1SG | 2678 |
| ATOM | 2678 | O | ALA | 324 | 21.283 | 6.680 | 41.753 | 1.00 | 0.18 | 1SG | 2679 |
| ATOM | 2679 | N | THR | 325 | 19.331 | 7.764 | 42.155 | 1.00 | 0.41 | 1SG | 2680 |
| ATOM | 2680 | CA | THR | 325 | 18.536 | 6.582 | 42.318 | 1.00 | 0.41 | 1SG | 2681 |
| ATOM | 2681 | CB | THR | 325 | 18.854 | 5.498 | 41.319 | 1.00 | 0.41 | 1SG | 2682 |
| ATOM | 2682 | OG1 | THR | 325 | 18.735 | 6.018 | 40.003 | 1.00 | 0.41 | 1SG | 2683 |
| ATOM | 2683 | CG2 | THR | 325 | 17.857 | 4.336 | 41.482 | 1.00 | 0.41 | 1SG | 2684 |
| ATOM | 2684 | C | THR | 325 | 18.784 | 6.098 | 43.712 | 1.00 | 0.41 | 1SG | 2685 |
| ATOM | 2685 | O | THR | 325 | 18.647 | 4.918 | 44.031 | 1.00 | 0.41 | 1SG | 2686 |
| ATOM | 2686 | N | ILE | 326 | 19.162 | 7.032 | 44.602 | 1.00 | 0.67 | 1SG | 2687 |
| ATOM | 2687 | CA | ILE | 326 | 19.350 | 6.648 | 45.967 | 1.00 | 0.67 | 1SG | 2688 |
| ATOM | 2688 | CB | ILE | 326 | 20.083 | 7.668 | 46.790 | 1.00 | 0.67 | 1SG | 2689 |
| ATOM | 2689 | CG2 | ILE | 326 | 19.197 | 8.921 | 46.886 | 1.00 | 0.67 | 1SG | 2690 |
| ATOM | 2690 | CG1 | ILE | 326 | 20.475 | 7.076 | 48.154 | 1.00 | 0.67 | 1SG | 2691 |
| ATOM | 2691 | CD1 | ILE | 326 | 21.514 | 5.959 | 48.058 | 1.00 | 0.67 | 1SG | 2692 |
| ATOM | 2692 | C | ILE | 326 | 17.980 | 6.506 | 46.534 | 1.00 | 0.67 | 1SG | 2693 |
| ATOM | 2693 | O | ILE | 326 | 17.036 | 7.099 | 46.015 | 1.00 | 0.67 | 1SG | 2694 |
| ATOM | 2694 | N | GLN | 327 | 17.845 | 5.723 | 47.622 | 1.00 | 0.76 | 1SG | 2695 |
| ATOM | 2695 | CA | GLN | 327 | 16.559 | 5.488 | 48.216 | 1.00 | 0.76 | 1SG | 2696 |
| ATOM | 2696 | CB | GLN | 327 | 15.969 | 6.735 | 48.893 | 1.00 | 0.76 | 1SG | 2697 |
| ATOM | 2697 | CG | GLN | 327 | 16.796 | 7.228 | 50.081 | 1.00 | 0.76 | 1SG | 2698 |
| ATOM | 2698 | CD | GLN | 327 | 16.109 | 8.456 | 50.660 | 1.00 | 0.76 | 1SG | 2699 |
| ATOM | 2699 | OE1 | GLN | 327 | 15.086 | 8.909 | 50.150 | 1.00 | 0.76 | 1SG | 2700 |
| ATOM | 2700 | NE2 | GLN | 327 | 16.690 | 9.017 | 51.754 | 1.00 | 0.76 | 1SG | 2701 |
| ATOM | 2701 | C | GLN | 327 | 15.630 | 5.035 | 47.139 | 1.00 | 0.76 | 1SG | 2702 |
| ATOM | 2702 | O | GLN | 327 | 14.907 | 5.829 | 46.544 | 1.00 | 0.76 | 1SG | 2703 |
| ATOM | 2703 | N | ARG | 328 | 15.624 | 3.720 | 46.854 | 1.00 | 0.91 | 1SG | 2704 |
| ATOM | 2704 | CA | ARG | 328 | 14.804 | 3.198 | 45.798 | 1.00 | 0.91 | 1SG | 2705 |
| ATOM | 2705 | CB | ARG | 328 | 14.859 | 1.666 | 45.685 | 1.00 | 0.91 | 1SG | 2706 |
| ATOM | 2706 | CG | ARG | 328 | 16.236 | 1.136 | 45.283 | 1.00 | 0.91 | 1SG | 2707 |
| ATOM | 2707 | CD | ARG | 328 | 16.514 | 1.223 | 43.781 | 1.00 | 0.91 | 1SG | 2708 |
| ATOM | 2708 | NE | ARG | 328 | 15.543 | 0.328 | 43.090 | 1.00 | 0.91 | 1SG | 2709 |
| ATOM | 2709 | CZ | ARG | 328 | 15.844 | −0.989 | 42.898 | 1.00 | 0.91 | 1SG | 2710 |
| ATOM | 2710 | NH1 | ARG | 328 | 17.034 | −1.488 | 43.345 | 1.00 | 0.91 | 1SG | 2711 |
| ATOM | 2711 | NH2 | ARG | 328 | 14.957 | −1.808 | 42.261 | 1.00 | 0.91 | 1SG | 2712 |
| ATOM | 2712 | C | ARG | 328 | 13.379 | 3.586 | 46.025 | 1.00 | 0.91 | 1SG | 2713 |
| ATOM | 2713 | O | ARG | 328 | 13.025 | 4.197 | 47.032 | 1.00 | 0.91 | 1SG | 2714 |
| ATOM | 2714 | N | ILE | 329 | 12.510 | 3.224 | 45.063 | 1.00 | 1.04 | 1SG | 2715 |
| ATOM | 2715 | CA | ILE | 329 | 11.128 | 3.577 | 45.162 | 1.00 | 1.04 | 1SG | 2716 |
| ATOM | 2716 | CB | ILE | 329 | 10.311 | 3.298 | 43.912 | 1.00 | 1.04 | 1SG | 2717 |
| ATOM | 2717 | CG2 | ILE | 329 | 10.512 | 1.864 | 43.406 | 1.00 | 1.04 | 1SG | 2718 |
| ATOM | 2718 | CG1 | ILE | 329 | 8.847 | 3.717 | 44.128 | 1.00 | 1.04 | 1SG | 2719 |
| ATOM | 2719 | CD1 | ILE | 329 | 8.022 | 3.718 | 42.843 | 1.00 | 1.04 | 1SG | 2720 |
| ATOM | 2720 | C | ILE | 329 | 10.518 | 2.962 | 46.383 | 1.00 | 1.04 | 1SG | 2721 |
| ATOM | 2721 | O | ILE | 329 | 9.591 | 3.553 | 46.935 | 1.00 | 1.04 | 1SG | 2722 |
| ATOM | 2722 | N | PRO | 330 | 10.951 | 1.828 | 46.872 | 1.00 | 1.01 | 1SG | 2723 |
| ATOM | 2723 | CA | PRO | 330 | 10.332 | 1.362 | 48.077 | 1.00 | 1.01 | 1SG | 2724 |
| ATOM | 2724 | CD | PRO | 330 | 11.400 | 0.723 | 46.041 | 1.00 | 1.01 | 1SG | 2725 |
| ATOM | 2725 | CB | PRO | 330 | 10.644 | −0.129 | 48.174 | 1.00 | 1.01 | 1SG | 2726 |
| ATOM | 2726 | CG | PRO | 330 | 10.869 | −0.550 | 46.714 | 1.00 | 1.01 | 1SG | 2727 |
| ATOM | 2727 | C | PRO | 330 | 10.883 | 2.147 | 49.218 | 1.00 | 1.01 | 1SG | 2728 |
| ATOM | 2728 | O | PRO | 330 | 10.267 | 2.166 | 50.282 | 1.00 | 1.01 | 1SG | 2729 |
| ATOM | 2729 | N | GLU | 331 | 12.048 | 2.792 | 49.019 | 1.00 | 0.93 | 1SG | 2730 |
| ATOM | 2730 | CA | GLU | 331 | 12.670 | 3.511 | 50.089 | 1.00 | 0.93 | 1SG | 2731 |
| ATOM | 2731 | CB | GLU | 331 | 14.031 | 4.108 | 49.694 | 1.00 | 0.93 | 1SG | 2732 |
| ATOM | 2732 | CG | GLU | 331 | 14.821 | 4.658 | 50.882 | 1.00 | 0.93 | 1SG | 2733 |
| ATOM | 2733 | CD | GLU | 331 | 15.367 | 3.473 | 51.666 | 1.00 | 0.93 | 1SG | 2734 |
| ATOM | 2734 | OE1 | GLU | 331 | 15.075 | 2.314 | 51.266 | 1.00 | 0.93 | 1SG | 2735 |
| ATOM | 2735 | OE2 | GLU | 331 | 16.084 | 3.710 | 52.675 | 1.00 | 0.93 | 1SG | 2736 |
| ATOM | 2736 | C | GLU | 331 | 11.783 | 4.639 | 50.499 | 1.00 | 0.93 | 1SG | 2737 |
| ATOM | 2737 | O | GLU | 331 | 11.488 | 4.795 | 51.683 | 1.00 | 0.93 | 1SG | 2738 |
| ATOM | 2738 | N | VAL | 332 | 11.302 | 5.426 | 49.515 | 1.00 | 0.75 | 1SG | 2739 |
| ATOM | 2739 | CA | VAL | 332 | 10.486 | 6.575 | 49.792 | 1.00 | 0.75 | 1SG | 2740 |
| ATOM | 2740 | CB | VAL | 332 | 10.813 | 7.367 | 51.050 | 1.00 | 0.75 | 1SG | 2741 |
| ATOM | 2741 | CG1 | VAL | 332 | 12.298 | 7.748 | 51.152 | 1.00 | 0.75 | 1SG | 2742 |
| ATOM | 2742 | CG2 | VAL | 332 | 9.858 | 8.571 | 51.096 | 1.00 | 0.75 | 1SG | 2743 |
| ATOM | 2743 | C | VAL | 332 | 10.417 | 7.451 | 48.578 | 1.00 | 0.75 | 1SG | 2744 |
| ATOM | 2744 | O | VAL | 332 | 9.337 | 7.558 | 47.998 | 1.00 | 0.75 | 1SG | 2745 |
| ATOM | 2745 | N | PRO | 333 | 11.476 | 8.100 | 48.149 | 1.00 | 0.49 | 1SG | 2746 |
| ATOM | 2746 | CA | PRO | 333 | 11.382 | 9.007 | 47.039 | 1.00 | 0.49 | 1SG | 2747 |
| ATOM | 2747 | CD | PRO | 333 | 12.814 | 7.525 | 48.205 | 1.00 | 0.49 | 1SG | 2748 |
| ATOM | 2748 | CB | PRO | 333 | 12.799 | 9.509 | 46.813 | 1.00 | 0.49 | 1SG | 2749 |
| ATOM | 2749 | CG | PRO | 333 | 13.637 | 8.270 | 47.140 | 1.00 | 0.49 | 1SG | 2750 |
| ATOM | 2750 | C | PRO | 333 | 10.879 | 8.320 | 45.802 | 1.00 | 0.49 | 1SG | 2751 |
| ATOM | 2751 | O | PRO | 333 | 10.735 | 9.012 | 44.798 | 1.00 | 0.49 | 1SG | 2752 |
| ATOM | 2752 | N | GLY | 334 | 10.632 | 6.992 | 45.816 | 1.00 | 0.29 | 1SG | 2753 |
| ATOM | 2753 | CA | GLY | 334 | 10.107 | 6.329 | 44.658 | 1.00 | 0.29 | 1SG | 2754 |
| ATOM | 2754 | C | GLY | 334 | 8.868 | 7.079 | 44.303 | 1.00 | 0.29 | 1SG | 2755 |
| ATOM | 2755 | O | GLY | 334 | 8.564 | 7.287 | 43.130 | 1.00 | 0.29 | 1SG | 2756 |
| ATOM | 2756 | N | SER | 335 | 8.100 | 7.487 | 45.326 | 1.00 | 0.39 | 1SG | 2757 |
| ATOM | 2757 | CA | SER | 335 | 7.015 | 8.377 | 45.056 | 1.00 | 0.39 | 1SG | 2758 |
| ATOM | 2758 | CB | SER | 335 | 5.779 | 8.145 | 45.941 | 1.00 | 0.39 | 1SG | 2759 |
| ATOM | 2759 | OG | SER | 335 | 5.207 | 6.875 | 45.663 | 1.00 | 0.39 | 1SG | 2760 |
| ATOM | 2760 | C | SER | 335 | 7.575 | 9.707 | 45.429 | 1.00 | 0.39 | 1SG | 2761 |
| ATOM | 2761 | O | SER | 335 | 7.745 | 10.001 | 46.610 | 1.00 | 0.39 | 1SG | 2762 |
| ATOM | 2762 | N | LEU | 336 | 7.909 | 10.545 | 44.430 | 1.00 | 0.55 | 1SG | 2763 |
| ATOM | 2763 | CA | LEU | 336 | 8.534 | 11.780 | 44.791 | 1.00 | 0.55 | 1SG | 2764 |
| ATOM | 2764 | CB | LEU | 336 | 10.067 | 11.671 | 44.799 | 1.00 | 0.55 | 1SG | 2765 |
| ATOM | 2765 | CG | LEU | 336 | 10.717 | 11.523 | 43.404 | 1.00 | 0.55 | 1SG | 2766 |
| ATOM | 2766 | CD2 | LEU | 336 | 10.871 | 12.865 | 42.675 | 1.00 | 0.55 | 1SG | 2767 |
| ATOM | 2767 | CD1 | LEU | 336 | 10.031 | 10.430 | 42.569 | 1.00 | 0.55 | 1SG | 2768 |
| ATOM | 2768 | C | LEU | 336 | 8.156 | 12.816 | 43.792 | 1.00 | 0.55 | 1SG | 2769 |
| ATOM | 2769 | O | LEU | 336 | 7.511 | 12.525 | 42.787 | 1.00 | 0.55 | 1SG | 2770 |
| ATOM | 2770 | N | SER | 337 | 8.538 | 14.076 | 44.068 | 1.00 | 0.45 | 1SG | 2771 |
| ATOM | 2771 | CA | SER | 337 | 8.266 | 15.125 | 43.136 | 1.00 | 0.45 | 1SG | 2772 |
| ATOM | 2772 | CB | SER | 337 | 7.122 | 16.058 | 43.572 | 1.00 | 0.45 | 1SG | 2773 |
| ATOM | 2773 | OG | SER | 337 | 5.893 | 15.346 | 43.614 | 1.00 | 0.45 | 1SG | 2774 |
| ATOM | 2774 | C | SER | 337 | 9.504 | 15.955 | 43.029 | 1.00 | 0.45 | 1SG | 2775 |
| ATOM | 2775 | O | SER | 337 | 10.222 | 16.151 | 44.008 | 1.00 | 0.45 | 1SG | 2776 |
| ATOM | 2776 | N | LEU | 338 | 9.803 | 16.445 | 41.812 | 1.00 | 0.36 | 1SG | 2777 |
| ATOM | 2777 | CA | LEU | 338 | 10.927 | 17.320 | 41.661 | 1.00 | 0.36 | 1SG | 2778 |
| ATOM | 2778 | CB | LEU | 338 | 11.813 | 17.019 | 40.439 | 1.00 | 0.36 | 1SG | 2779 |
| ATOM | 2779 | CG | LEU | 338 | 12.442 | 15.615 | 40.449 | 1.00 | 0.36 | 1SG | 2780 |
| ATOM | 2780 | CD2 | LEU | 338 | 13.558 | 15.495 | 39.399 | 1.00 | 0.36 | 1SG | 2781 |
| ATOM | 2781 | CD1 | LEU | 338 | 11.365 | 14.530 | 40.297 | 1.00 | 0.36 | 1SG | 2782 |
| ATOM | 2782 | C | LEU | 338 | 10.325 | 18.661 | 41.416 | 1.00 | 0.36 | 1SG | 2783 |
| ATOM | 2783 | O | LEU | 338 | 9.503 | 18.819 | 40.516 | 1.00 | 0.36 | 1SG | 2784 |
| ATOM | 2784 | N | SER | 339 | 10.702 | 19.669 | 42.219 | 1.00 | 0.37 | 1SG | 2785 |
| ATOM | 2785 | CA | SER | 339 | 10.093 | 20.946 | 42.009 | 1.00 | 0.37 | 1SG | 2786 |
| ATOM | 2786 | CB | SER | 339 | 9.577 | 21.606 | 43.300 | 1.00 | 0.37 | 1SG | 2787 |
| ATOM | 2787 | OG | SER | 339 | 10.660 | 21.889 | 44.174 | 1.00 | 0.37 | 1SG | 2788 |
| ATOM | 2788 | C | SER | 339 | 11.111 | 21.854 | 41.412 | 1.00 | 0.37 | 1SG | 2789 |
| ATOM | 2789 | O | SER | 339 | 12.244 | 21.939 | 41.885 | 1.00 | 0.37 | 1SG | 2790 |
| ATOM | 2790 | N | TYR | 340 | 10.729 | 22.548 | 40.325 | 1.00 | 0.57 | 1SG | 2791 |
| ATOM | 2791 | CA | TYR | 340 | 11.642 | 23.474 | 39.735 | 1.00 | 0.57 | 1SG | 2792 |
| ATOM | 2792 | CB | TYR | 340 | 11.890 | 23.256 | 38.229 | 1.00 | 0.57 | 1SG | 2793 |
| ATOM | 2793 | CG | TYR | 340 | 10.611 | 23.364 | 37.472 | 1.00 | 0.57 | 1SG | 2794 |
| ATOM | 2794 | CD1 | TYR | 340 | 9.832 | 22.249 | 37.264 | 1.00 | 0.57 | 1SG | 2795 |
| ATOM | 2795 | CD2 | TYR | 340 | 10.192 | 24.571 | 36.964 | 1.00 | 0.57 | 1SG | 2796 |
| ATOM | 2796 | CE1 | TYR | 340 | 8.652 | 22.330 | 36.566 | 1.00 | 0.57 | 1SG | 2797 |
| ATOM | 2797 | CE2 | TYR | 340 | 9.011 | 24.657 | 36.264 | 1.00 | 0.57 | 1SG | 2798 |
| ATOM | 2798 | CZ | TYR | 340 | 8.241 | 23.538 | 36.061 | 1.00 | 0.57 | 1SG | 2799 |
| ATOM | 2799 | OH | TYR | 340 | 7.031 | 23.631 | 35.342 | 1.00 | 0.57 | 1SG | 2800 |
| ATOM | 2800 | C | TYR | 340 | 11.090 | 24.837 | 39.978 | 1.00 | 0.57 | 1SG | 2801 |
| ATOM | 2801 | O | TYR | 340 | 9.876 | 25.034 | 40.024 | 1.00 | 0.57 | 1SG | 2802 |
| ATOM | 2802 | N | LYS | 341 | 11.992 | 25.815 | 40.173 | 1.00 | 0.85 | 1SG | 2803 |
| ATOM | 2803 | CA | LYS | 341 | 11.581 | 27.145 | 40.501 | 1.00 | 0.85 | 1SG | 2804 |
| ATOM | 2804 | CB | LYS | 341 | 12.758 | 28.042 | 40.931 | 1.00 | 0.85 | 1SG | 2805 |
| ATOM | 2805 | CG | LYS | 341 | 12.346 | 29.340 | 41.629 | 1.00 | 0.85 | 1SG | 2806 |
| ATOM | 2806 | CD | LYS | 341 | 13.503 | 30.055 | 42.330 | 1.00 | 0.85 | 1SG | 2807 |
| ATOM | 2807 | CE | LYS | 341 | 13.088 | 31.352 | 43.023 | 1.00 | 0.85 | 1SG | 2808 |
| ATOM | 2808 | NZ | LYS | 341 | 12.661 | 32.349 | 42.017 | 1.00 | 0.85 | 1SG | 2809 |
| ATOM | 2809 | C | LYS | 341 | 10.926 | 27.749 | 39.303 | 1.00 | 0.85 | 1SG | 2810 |
| ATOM | 2810 | O | LYS | 341 | 10.620 | 27.065 | 38.328 | 1.00 | 0.85 | 1SG | 2811 |
| ATOM | 2811 | N | TYR | 342 | 10.676 | 29.068 | 39.372 | 1.00 | 0.89 | 1SG | 2812 |
| ATOM | 2812 | CA | TYR | 342 | 10.049 | 29.795 | 38.312 | 1.00 | 0.89 | 1SG | 2813 |
| ATOM | 2813 | CB | TYR | 342 | 9.748 | 31.254 | 38.704 | 1.00 | 0.89 | 1SG | 2814 |
| ATOM | 2814 | CG | TYR | 342 | 9.068 | 31.924 | 37.558 | 1.00 | 0.89 | 1SG | 2815 |
| ATOM | 2815 | CD1 | TYR | 342 | 9.790 | 32.510 | 36.544 | 1.00 | 0.89 | 1SG | 2816 |
| ATOM | 2816 | CD2 | TYR | 342 | 7.694 | 31.963 | 37.506 | 1.00 | 0.89 | 1SG | 2817 |
| ATOM | 2817 | CE1 | TYR | 342 | 9.149 | 33.124 | 35.494 | 1.00 | 0.89 | 1SG | 2818 |
| ATOM | 2818 | CE2 | TYR | 342 | 7.047 | 32.576 | 36.459 | 1.00 | 0.89 | 1SG | 2819 |
| ATOM | 2819 | CZ | TYR | 342 | 7.775 | 33.157 | 35.450 | 1.00 | 0.89 | 1SG | 2820 |
| ATOM | 2820 | OH | TYR | 342 | 7.114 | 33.787 | 34.374 | 1.00 | 0.89 | 1SG | 2821 |
| ATOM | 2821 | C | TYR | 342 | 11.022 | 29.808 | 37.183 | 1.00 | 0.89 | 1SG | 2822 |
| ATOM | 2822 | O | TYR | 342 | 10.651 | 29.959 | 36.020 | 1.00 | 0.89 | 1SG | 2823 |
| ATOM | 2823 | N | ASP | 343 | 12.315 | 29.642 | 37.509 | 1.00 | 0.56 | 1SG | 2824 |
| ATOM | 2824 | CA | ASP | 343 | 13.318 | 29.679 | 36.491 | 1.00 | 0.56 | 1SG | 2825 |
| ATOM | 2825 | CB | ASP | 343 | 14.748 | 29.475 | 37.027 | 1.00 | 0.56 | 1SG | 2826 |
| ATOM | 2826 | CG | ASP | 343 | 15.737 | 29.743 | 35.898 | 1.00 | 0.56 | 1SG | 2827 |
| ATOM | 2827 | OD1 | ASP | 343 | 15.281 | 30.056 | 34.766 | 1.00 | 0.56 | 1SG | 2828 |
| ATOM | 2828 | OD2 | ASP | 343 | 16.966 | 29.646 | 36.159 | 1.00 | 0.56 | 1SG | 2829 |
| ATOM | 2829 | C | ASP | 343 | 13.038 | 28.593 | 35.509 | 1.00 | 0.56 | 1SG | 2830 |
| ATOM | 2830 | O | ASP | 343 | 12.879 | 27.428 | 35.869 | 1.00 | 0.56 | 1SG | 2831 |
| ATOM | 2831 | N | THE | 344 | 12.951 | 28.980 | 34.223 | 1.00 | 0.27 | 1SG | 2832 |
| ATOM | 2832 | CA | THR | 344 | 12.747 | 28.052 | 33.156 | 1.00 | 0.27 | 1SG | 2833 |
| ATOM | 2833 | CB | THR | 344 | 12.606 | 28.723 | 31.823 | 1.00 | 0.27 | 1SG | 2834 |
| ATOM | 2834 | OG1 | THR | 344 | 13.792 | 29.438 | 31.507 | 1.00 | 0.27 | 1SG | 2835 |
| ATOM | 2835 | CG2 | THR | 344 | 11.414 | 29.690 | 31.887 | 1.00 | 0.27 | 1SG | 2836 |
| ATOM | 2836 | C | THR | 344 | 13.972 | 27.204 | 33.110 | 1.00 | 0.27 | 1SG | 2837 |
| ATOM | 2837 | O | THR | 344 | 13.914 | 26.011 | 32.823 | 1.00 | 0.27 | 1SG | 2838 |
| ATOM | 2838 | N | SER | 345 | 15.130 | 27.814 | 33.411 | 1.00 | 0.35 | 1SG | 2839 |
| ATOM | 2839 | CA | SER | 345 | 16.372 | 27.106 | 33.374 | 1.00 | 0.35 | 1SG | 2840 |
| ATOM | 2840 | CB | SER | 345 | 17.573 | 27.976 | 33.781 | 1.00 | 0.35 | 1SG | 2841 |
| ATOM | 2841 | OG | SER | 345 | 18.771 | 27.217 | 33.717 | 1.00 | 0.35 | 1SG | 2842 |
| ATOM | 2842 | C | SER | 345 | 16.289 | 25.975 | 34.347 | 1.00 | 0.35 | 1SG | 2843 |
| ATOM | 2843 | O | SER | 345 | 16.767 | 24.876 | 34.074 | 1.00 | 0.35 | 1SG | 2844 |
| ATOM | 2844 | N | ASP | 346 | 15.669 | 26.212 | 35.516 | 1.00 | 0.59 | 1SG | 2845 |
| ATOM | 2845 | CA | ASP | 346 | 15.600 | 25.173 | 36.501 | 1.00 | 0.59 | 1SG | 2846 |
| ATOM | 2846 | CB | ASP | 346 | 14.870 | 25.623 | 37.781 | 1.00 | 0.59 | 1SG | 2847 |
| ATOM | 2847 | CG | ASP | 346 | 15.049 | 24.546 | 38.842 | 1.00 | 0.59 | 1SG | 2848 |
| ATOM | 2848 | OD1 | ASP | 346 | 15.593 | 23.461 | 38.504 | 1.00 | 0.59 | 1SG | 2849 |
| ATOM | 2849 | OD2 | ASP | 346 | 14.649 | 24.800 | 40.010 | 1.00 | 0.59 | 1SG | 2850 |
| ATOM | 2850 | C | ASP | 346 | 14.842 | 24.021 | 35.924 | 1.00 | 0.59 | 1SG | 2851 |
| ATOM | 2851 | O | ASP | 346 | 15.273 | 22.873 | 36.027 | 1.00 | 0.59 | 1SG | 2852 |
| ATOM | 2852 | N | MET | 347 | 13.694 | 24.298 | 35.278 | 1.00 | 0.75 | 1SG | 2853 |
| ATOM | 2853 | CA | MET | 347 | 12.903 | 23.230 | 34.740 | 1.00 | 0.75 | 1SG | 2854 |
| ATOM | 2854 | CB | MET | 347 | 11.508 | 23.684 | 34.256 | 1.00 | 0.75 | 1SG | 2855 |
| ATOM | 2855 | CG | MET | 347 | 11.473 | 24.837 | 33.249 | 1.00 | 0.75 | 1SG | 2856 |
| ATOM | 2856 | SD | MET | 347 | 11.916 | 24.403 | 31.542 | 1.00 | 0.75 | 1SG | 2857 |
| ATOM | 2857 | CE | MET | 347 | 11.699 | 26.079 | 30.874 | 1.00 | 0.75 | 1SG | 2858 |
| ATOM | 2858 | C | MET | 347 | 13.654 | 22.550 | 33.639 | 1.00 | 0.75 | 1SG | 2859 |
| ATOM | 2859 | O | MET | 347 | 13.642 | 21.324 | 33.541 | 1.00 | 0.75 | 1SG | 2860 |
| ATOM | 2860 | N | GLN | 348 | 14.351 | 23.321 | 32.786 | 1.00 | 0.59 | 1SG | 2861 |
| ATOM | 2861 | CA | GLN | 348 | 15.057 | 22.719 | 31.695 | 1.00 | 0.59 | 1SG | 2862 |
| ATOM | 2862 | CB | GLN | 348 | 15.675 | 23.748 | 30.727 | 1.00 | 0.59 | 1SG | 2863 |
| ATOM | 2863 | CG | GLN | 348 | 16.755 | 24.643 | 31.340 | 1.00 | 0.59 | 1SG | 2864 |
| ATOM | 2864 | CD | GLN | 348 | 18.083 | 23.897 | 31.308 | 1.00 | 0.59 | 1SG | 2865 |
| ATOM | 2865 | OE1 | GLN | 348 | 18.204 | 22.835 | 30.699 | 1.00 | 0.59 | 1SG | 2866 |
| ATOM | 2866 | NE2 | GLN | 348 | 19.114 | 24.471 | 31.984 | 1.00 | 0.59 | 1SG | 2867 |
| ATOM | 2867 | C | GLN | 348 | 16.155 | 21.864 | 32.246 | 1.00 | 0.59 | 1SG | 2868 |
| ATOM | 2868 | O | GLN | 348 | 16.407 | 20.769 | 31.745 | 1.00 | 0.59 | 1SG | 2869 |
| ATOM | 2869 | N | ALA | 349 | 16.832 | 22.340 | 33.307 | 1.00 | 0.23 | 1SG | 2870 |
| ATOM | 2870 | CA | ALA | 349 | 17.942 | 21.616 | 33.857 | 1.00 | 0.23 | 1SG | 2871 |
| ATOM | 2871 | CB | ALA | 349 | 18.617 | 22.357 | 35.023 | 1.00 | 0.23 | 1SG | 2872 |
| ATOM | 2872 | C | ALA | 349 | 17.465 | 20.299 | 34.381 | 1.00 | 0.23 | 1SG | 2873 |
| ATOM | 2873 | O | ALA | 349 | 18.071 | 19.263 | 34.116 | 1.00 | 0.23 | 1SG | 2874 |
| ATOM | 2874 | N | ILE | 350 | 16.349 | 20.308 | 35.131 | 1.00 | 0.09 | 1SG | 2875 |
| ATOM | 2875 | CA | ILE | 350 | 15.827 | 19.105 | 35.712 | 1.00 | 0.09 | 1SG | 2876 |
| ATOM | 2876 | CB | ILE | 350 | 14.635 | 19.354 | 36.588 | 1.00 | 0.09 | 1SG | 2877 |
| ATOM | 2877 | CG2 | ILE | 350 | 14.070 | 17.991 | 37.021 | 1.00 | 0.09 | 1SG | 2878 |
| ATOM | 2878 | CG1 | ILE | 350 | 15.021 | 20.266 | 37.766 | 1.00 | 0.09 | 1SG | 2879 |
| ATOM | 2879 | CD1 | ILE | 350 | 13.818 | 20.799 | 38.543 | 1.00 | 0.09 | 1SG | 2880 |
| ATOM | 2880 | C | ILE | 350 | 15.394 | 18.203 | 34.604 | 1.00 | 0.09 | 1SG | 2881 |
| ATOM | 2881 | O | ILE | 350 | 15.543 | 16.985 | 34.682 | 1.00 | 0.09 | 1SG | 2882 |
| ATOM | 2882 | N | ALA | 351 | 14.864 | 18.802 | 33.525 | 1.00 | 0.19 | 1SG | 2883 |
| ATOM | 2883 | CA | ALA | 351 | 14.339 | 18.080 | 32.404 | 1.00 | 0.19 | 1SG | 2884 |
| ATOM | 2884 | CB | ALA | 351 | 13.782 | 19.004 | 31.309 | 1.00 | 0.19 | 1SG | 2885 |
| ATOM | 2885 | C | ALA | 351 | 15.428 | 17.264 | 31.789 | 1.00 | 0.19 | 1SG | 2886 |
| ATOM | 2886 | O | ALA | 351 | 15.177 | 16.175 | 31.276 | 1.00 | 0.19 | 1SG | 2887 |
| ATOM | 2887 | N | ARG | 352 | 16.676 | 17.761 | 31.846 | 1.00 | 0.39 | 1SG | 2888 |
| ATOM | 2888 | CA | ARG | 352 | 17.772 | 17.086 | 31.213 | 1.00 | 0.39 | 1SG | 2889 |
| ATOM | 2889 | CB | ARG | 352 | 19.139 | 17.726 | 31.516 | 1.00 | 0.39 | 1SG | 2890 |
| ATOM | 2890 | CG | ARG | 352 | 19.349 | 19.092 | 30.863 | 1.00 | 0.39 | 1SG | 2891 |
| ATOM | 2891 | CD | ARG | 352 | 20.755 | 19.660 | 31.061 | 1.00 | 0.39 | 1SG | 2892 |
| ATOM | 2892 | NE | ARG | 352 | 20.806 | 20.963 | 30.342 | 1.00 | 0.39 | 1SG | 2893 |
| ATOM | 2893 | CZ | ARG | 352 | 21.147 | 20.999 | 29.021 | 1.00 | 0.39 | 1SG | 2894 |
| ATOM | 2894 | NH1 | ARG | 352 | 21.457 | 19.843 | 28.365 | 1.00 | 0.39 | 1SG | 2895 |
| ATOM | 2895 | NH2 | ARG | 352 | 21.178 | 22.192 | 28.357 | 1.00 | 0.39 | 1SG | 2896 |
| ATOM | 2896 | C | ARG | 352 | 17.836 | 15.685 | 31.727 | 1.00 | 0.39 | 1SG | 2897 |
| ATOM | 2897 | O | ARG | 352 | 18.177 | 14.766 | 30.985 | 1.00 | 0.39 | 1SG | 2898 |
| ATOM | 2898 | N | PHE | 353 | 17.487 | 15.478 | 33.008 | 1.00 | 0.55 | 1SG | 2899 |
| ATOM | 2899 | CA | PHE | 353 | 17.610 | 14.173 | 33.580 | 1.00 | 0.55 | 1SG | 2900 |
| ATOM | 2900 | CB | PHE | 353 | 17.136 | 14.130 | 35.039 | 1.00 | 0.55 | 1SG | 2901 |
| ATOM | 2901 | CG | PHE | 353 | 18.096 | 14.971 | 35.812 | 1.00 | 0.55 | 1SG | 2902 |
| ATOM | 2902 | CD1 | PHE | 353 | 19.275 | 14.437 | 36.282 | 1.00 | 0.55 | 1SG | 2903 |
| ATOM | 2903 | CD2 | PHE | 353 | 17.822 | 16.296 | 36.060 | 1.00 | 0.55 | 1SG | 2904 |
| ATOM | 2904 | CE1 | PHE | 353 | 20.163 | 15.211 | 36.994 | 1.00 | 0.55 | 1SG | 2905 |
| ATOM | 2905 | CE2 | PHE | 353 | 18.705 | 17.075 | 36.771 | 1.00 | 0.55 | 1SG | 2906 |
| ATOM | 2906 | CZ | PHE | 353 | 19.878 | 16.532 | 37.240 | 1.00 | 0.55 | 1SG | 2907 |
| ATOM | 2907 | C | PHE | 353 | 16.806 | 13.203 | 32.776 | 1.00 | 0.55 | 1SG | 2908 |
| ATOM | 2908 | O | PHE | 353 | 17.284 | 12.116 | 32.455 | 1.00 | 0.55 | 1SG | 2909 |
| ATOM | 2909 | N | VAL | 354 | 15.558 | 13.549 | 32.414 | 1.00 | 0.57 | 1SG | 2910 |
| ATOM | 2910 | CA | VAL | 354 | 14.847 | 12.584 | 31.631 | 1.00 | 0.57 | 1SG | 2911 |
| ATOM | 2911 | CB | VAL | 354 | 14.420 | 11.431 | 32.491 | 1.00 | 0.57 | 1SG | 2912 |
| ATOM | 2912 | CG1 | VAL | 354 | 13.624 | 11.983 | 33.683 | 1.00 | 0.57 | 1SG | 2913 |
| ATOM | 2913 | CG2 | VAL | 354 | 13.639 | 10.424 | 31.641 | 1.00 | 0.57 | 1SG | 2914 |
| ATOM | 2914 | C | VAL | 354 | 13.637 | 13.233 | 31.034 | 1.00 | 0.57 | 1SG | 2915 |
| ATOM | 2915 | O | VAL | 354 | 12.501 | 12.888 | 31.356 | 1.00 | 0.57 | 1SG | 2916 |
| ATOM | 2916 | N | LYS | 355 | 13.823 | 14.181 | 30.100 | 1.00 | 0.46 | 1SG | 2917 |
| ATOM | 2917 | CA | LYS | 355 | 12.608 | 14.758 | 29.615 | 1.00 | 0.46 | 1SG | 2918 |
| ATOM | 2918 | CB | LYS | 355 | 12.018 | 15.844 | 30.534 | 1.00 | 0.46 | 1SG | 2919 |
| ATOM | 2919 | CG | LYS | 355 | 11.554 | 15.392 | 31.919 | 1.00 | 0.46 | 1SG | 2920 |
| ATOM | 2920 | CD | LYS | 355 | 11.335 | 16.572 | 32.865 | 1.00 | 0.46 | 1SG | 2921 |
| ATOM | 2921 | CE | LYS | 355 | 10.388 | 17.624 | 32.291 | 1.00 | 0.46 | 1SG | 2922 |
| ATOM | 2922 | NZ | LYS | 355 | 10.537 | 18.891 | 33.033 | 1.00 | 0.46 | 1SG | 2923 |
| ATOM | 2923 | C | LYS | 355 | 12.862 | 15.485 | 28.342 | 1.00 | 0.46 | 1SG | 2924 |
| ATOM | 2924 | O | LYS | 355 | 13.986 | 15.875 | 28.030 | 1.00 | 0.46 | 1SG | 2925 |
| ATOM | 2925 | N | TRP | 356 | 11.776 | 15.682 | 27.574 | 1.00 | 0.36 | 1SG | 2926 |
| ATOM | 2926 | CA | TRP | 356 | 11.818 | 16.496 | 26.401 | 1.00 | 0.36 | 1SG | 2927 |
| ATOM | 2927 | CB | TRP | 356 | 10.494 | 16.465 | 25.622 | 1.00 | 0.36 | 1SG | 2928 |
| ATOM | 2928 | CG | TRP | 356 | 10.120 | 15.104 | 25.080 | 1.00 | 0.36 | 1SG | 2929 |
| ATOM | 2929 | CD2 | TRP | 356 | 9.890 | 14.805 | 23.694 | 1.00 | 0.36 | 1SG | 2930 |
| ATOM | 2930 | CD1 | TRP | 356 | 9.876 | 13.953 | 25.769 | 1.00 | 0.36 | 1SG | 2931 |
| ATOM | 2931 | NE1 | TRP | 356 | 9.521 | 12.950 | 24.900 | 1.00 | 0.36 | 1SG | 2932 |
| ATOM | 2932 | CE2 | TRP | 356 | 9.522 | 13.462 | 23.620 | 1.00 | 0.36 | 1SG | 2933 |
| ATOM | 2933 | CE3 | TRP | 356 | 9.972 | 15.586 | 22.577 | 1.00 | 0.36 | 1SG | 2934 |
| ATOM | 2934 | CZ2 | TRP | 356 | 9.228 | 12.877 | 22.422 | 1.00 | 0.36 | 1SG | 2935 |
| ATOM | 2935 | CZ3 | TRP | 356 | 9.682 | 14.990 | 21.369 | 1.00 | 0.36 | 1SG | 2936 |
| ATOM | 2936 | CH2 | TRP | 356 | 9.318 | 13.662 | 21.294 | 1.00 | 0.36 | 1SG | 2937 |
| ATOM | 2937 | C | TRP | 356 | 11.959 | 17.877 | 26.960 | 1.00 | 0.36 | 1SG | 2938 |
| ATOM | 2938 | O | TRP | 356 | 11.166 | 18.285 | 27.806 | 1.00 | 0.36 | 1SG | 2939 |
| ATOM | 2939 | N | GLN | 357 | 12.998 | 18.612 | 26.527 | 1.00 | 0.44 | 1SG | 2940 |
| ATOM | 2940 | CA | GLN | 357 | 13.310 | 19.912 | 27.055 | 1.00 | 0.44 | 1SG | 2941 |
| ATOM | 2941 | CB | GLN | 357 | 14.676 | 20.426 | 26.570 | 1.00 | 0.44 | 1SG | 2942 |
| ATOM | 2942 | CG | GLN | 357 | 15.861 | 19.664 | 27.157 | 1.00 | 0.44 | 1SG | 2943 |
| ATOM | 2943 | CD | GLN | 357 | 15.976 | 20.081 | 28.614 | 1.00 | 0.44 | 1SG | 2944 |
| ATOM | 2944 | OE1 | GLN | 357 | 15.214 | 20.920 | 29.093 | 1.00 | 0.44 | 1SG | 2945 |
| ATOM | 2945 | NE2 | GLN | 357 | 16.955 | 19.482 | 29.340 | 1.00 | 0.44 | 1SG | 2946 |
| ATOM | 2946 | C | GLN | 357 | 12.315 | 20.975 | 26.693 | 1.00 | 0.44 | 1SG | 2947 |
| ATOM | 2947 | O | GLN | 357 | 11.932 | 21.770 | 27.549 | 1.00 | 0.44 | 1SG | 2948 |
| ATOM | 2948 | N | TYR | 358 | 11.862 | 21.031 | 25.425 | 1.00 | 0.54 | 1SG | 2949 |
| ATOM | 2949 | CA | TYR | 358 | 11.057 | 22.155 | 25.025 | 1.00 | 0.54 | 1SG | 2950 |
| ATOM | 2950 | CB | TYR | 358 | 11.031 | 22.394 | 23.504 | 1.00 | 0.54 | 1SG | 2951 |
| ATOM | 2951 | CG | TYR | 358 | 10.528 | 21.167 | 22.832 | 1.00 | 0.54 | 1SG | 2952 |
| ATOM | 2952 | CD1 | TYR | 358 | 9.179 | 20.943 | 22.698 | 1.00 | 0.54 | 1SG | 2953 |
| ATOM | 2953 | CD2 | TYR | 358 | 11.414 | 20.243 | 22.326 | 1.00 | 0.54 | 1SG | 2954 |
| ATOM | 2954 | CE1 | TYR | 358 | 8.724 | 19.808 | 22.073 | 1.00 | 0.54 | 1SG | 2955 |
| ATOM | 2955 | CE2 | TYR | 358 | 10.962 | 19.106 | 21.701 | 1.00 | 0.54 | 1SG | 2956 |
| ATOM | 2956 | CZ | TYR | 358 | 9.612 | 18.888 | 21.574 | 1.00 | 0.54 | 1SG | 2957 |
| ATOM | 2957 | OH | TYR | 358 | 9.140 | 17.723 | 20.933 | 1.00 | 0.54 | 1SG | 2958 |
| ATOM | 2958 | C | TYR | 358 | 9.660 | 22.058 | 25.543 | 1.00 | 0.54 | 1SG | 2959 |
| ATOM | 2959 | O | TYR | 358 | 9.207 | 20.999 | 25.974 | 1.00 | 0.54 | 1SG | 2960 |
| ATOM | 2960 | N | PHE | 359 | 8.955 | 23.212 | 25.539 | 1.00 | 0.59 | 1SG | 2961 |
| ATOM | 2961 | CA | PHE | 359 | 7.611 | 23.271 | 26.039 | 1.00 | 0.59 | 1SG | 2962 |
| ATOM | 2962 | CB | PHE | 359 | 7.497 | 24.057 | 27.356 | 1.00 | 0.59 | 1SG | 2963 |
| ATOM | 2963 | CG | PHE | 359 | 8.241 | 23.325 | 28.418 | 1.00 | 0.59 | 1SG | 2964 |
| ATOM | 2964 | CD1 | PHE | 359 | 9.605 | 23.460 | 28.527 | 1.00 | 0.59 | 1SG | 2965 |
| ATOM | 2965 | CD2 | PHE | 359 | 7.581 | 22.496 | 29.294 | 1.00 | 0.59 | 1SG | 2966 |
| ATOM | 2966 | CE1 | PHE | 359 | 10.295 | 22.785 | 29.503 | 1.00 | 0.59 | 1SG | 2967 |
| ATOM | 2967 | CE2 | PHE | 359 | 8.270 | 21.821 | 30.273 | 1.00 | 0.59 | 1SG | 2968 |
| ATOM | 2968 | CZ | PHE | 359 | 9.631 | 21.964 | 30.381 | 1.00 | 0.59 | 1SG | 2969 |
| ATOM | 2969 | C | PHE | 359 | 6.779 | 24.023 | 25.054 | 1.00 | 0.59 | 1SG | 2970 |
| ATOM | 2970 | O | PHE | 359 | 7.230 | 25.001 | 24.460 | 1.00 | 0.59 | 1SG | 2971 |
| ATOM | 2971 | N | GLU | 360 | 5.528 | 23.573 | 24.840 | 1.00 | 0.55 | 1SG | 2972 |
| ATOM | 2972 | CA | GLU | 360 | 4.680 | 24.338 | 23.981 | 1.00 | 0.55 | 1SG | 2973 |
| ATOM | 2973 | CB | GLU | 360 | 4.223 | 23.616 | 22.708 | 1.00 | 0.55 | 1SG | 2974 |
| ATOM | 2974 | CG | GLU | 360 | 3.535 | 24.580 | 21.740 | 1.00 | 0.55 | 1SG | 2975 |
| ATOM | 2975 | CD | GLU | 360 | 4.582 | 25.582 | 21.276 | 1.00 | 0.55 | 1SG | 2976 |
| ATOM | 2976 | OE1 | GLU | 360 | 5.692 | 25.137 | 20.879 | 1.00 | 0.55 | 1SG | 2977 |
| ATOM | 2977 | OE2 | GLU | 360 | 4.292 | 26.807 | 21.328 | 1.00 | 0.55 | 1SG | 2978 |
| ATOM | 2978 | C | GLU | 360 | 3.465 | 24.687 | 24.769 | 1.00 | 0.55 | 1SG | 2979 |
| ATOM | 2979 | O | GLU | 360 | 2.799 | 23.818 | 25.330 | 1.00 | 0.55 | 1SG | 2980 |
| ATOM | 2980 | N | GLY | 361 | 3.153 | 25.992 | 24.846 | 1.00 | 0.45 | 1SG | 2981 |
| ATOM | 2981 | CA | GLY | 361 | 1.992 | 26.394 | 25.575 | 1.00 | 0.45 | 1SG | 2982 |
| ATOM | 2982 | C | GLY | 361 | 2.247 | 26.141 | 27.025 | 1.00 | 0.45 | 1SG | 2983 |
| ATOM | 2983 | O | GLY | 361 | 1.299 | 25.963 | 27.788 | 1.00 | 0.45 | 1SG | 2984 |
| ATOM | 2984 | N | ASP | 362 | 3.540 | 26.123 | 27.425 | 1.00 | 0.49 | 1SG | 2985 |
| ATOM | 2985 | CA | ASP | 362 | 3.942 | 25.932 | 28.795 | 1.00 | 0.49 | 1SG | 2986 |
| ATOM | 2986 | CB | ASP | 362 | 3.002 | 26.641 | 29.800 | 1.00 | 0.49 | 1SG | 2987 |
| ATOM | 2987 | CG | ASP | 362 | 3.336 | 26.255 | 31.238 | 1.00 | 0.49 | 1SG | 2988 |
| ATOM | 2988 | OD1 | ASP | 362 | 4.550 | 26.165 | 31.564 | 1.00 | 0.49 | 1SG | 2989 |
| ATOM | 2989 | OD2 | ASP | 362 | 2.376 | 26.022 | 32.020 | 1.00 | 0.49 | 1SG | 2990 |
| ATOM | 2990 | C | ASP | 362 | 3.939 | 24.474 | 29.129 | 1.00 | 0.49 | 1SG | 2991 |
| ATOM | 2991 | O | ASP | 362 | 4.509 | 24.061 | 30.138 | 1.00 | 0.49 | 1SG | 2992 |
| ATOM | 2992 | N | VAL | 363 | 3.368 | 23.628 | 28.257 | 1.00 | 0.52 | 1SG | 2993 |
| ATOM | 2993 | CA | VAL | 363 | 3.258 | 22.259 | 28.654 | 1.00 | 0.52 | 1SG | 2994 |
| ATOM | 2994 | CB | VAL | 363 | 2.025 | 21.596 | 28.123 | 1.00 | 0.52 | 1SG | 2995 |
| ATOM | 2995 | CG1 | VAL | 363 | 2.145 | 21.471 | 26.597 | 1.00 | 0.52 | 1SG | 2996 |
| ATOM | 2996 | CG2 | VAL | 363 | 1.848 | 20.260 | 28.849 | 1.00 | 0.52 | 1SG | 2997 |
| ATOM | 2997 | C | VAL | 363 | 4.442 | 21.484 | 28.178 | 1.00 | 0.52 | 1SG | 2998 |
| ATOM | 2998 | O | VAL | 363 | 4.854 | 21.580 | 27.022 | 1.00 | 0.52 | 1SG | 2999 |
| ATOM | 2999 | N | SER | 364 | 5.024 | 20.685 | 29.096 | 1.00 | 0.56 | 1SG | 3000 |
| ATOM | 3000 | CA | SER | 364 | 6.153 | 19.859 | 28.783 | 1.00 | 0.56 | 1SG | 3001 |
| ATOM | 3001 | CB | SER | 364 | 6.836 | 19.260 | 30.024 | 1.00 | 0.56 | 1SG | 3002 |
| ATOM | 3002 | OG | SER | 364 | 7.940 | 18.456 | 29.636 | 1.00 | 0.56 | 1SG | 3003 |
| ATOM | 3003 | C | SER | 364 | 5.637 | 18.715 | 27.974 | 1.00 | 0.56 | 1SG | 3004 |
| ATOM | 3004 | O | SER | 364 | 4.440 | 18.432 | 27.971 | 1.00 | 0.56 | 1SG | 3005 |
| ATOM | 3005 | N | LYS | 365 | 6.538 | 18.020 | 27.250 | 1.00 | 0.60 | 1SG | 3006 |
| ATOM | 3006 | CA | LYS | 365 | 6.071 | 16.938 | 26.439 | 1.00 | 0.60 | 1SG | 3007 |
| ATOM | 3007 | CB | LYS | 365 | 7.131 | 16.376 | 25.475 | 1.00 | 0.60 | 1SG | 3008 |
| ATOM | 3008 | CG | LYS | 365 | 7.883 | 17.465 | 24.706 | 1.00 | 0.60 | 1SG | 3009 |
| ATOM | 3009 | CD | LYS | 365 | 6.976 | 18.504 | 24.050 | 1.00 | 0.60 | 1SG | 3010 |
| ATOM | 3010 | CE | LYS | 365 | 6.656 | 19.686 | 24.970 | 1.00 | 0.60 | 1SG | 3011 |
| ATOM | 3011 | NZ | LYS | 365 | 6.139 | 20.827 | 24.181 | 1.00 | 0.60 | 1SG | 3012 |
| ATOM | 3012 | C | LYS | 365 | 5.710 | 15.830 | 27.366 | 1.00 | 0.60 | 1SG | 3013 |
| ATOM | 3013 | O | LYS | 365 | 6.532 | 15.431 | 28.191 | 1.00 | 0.60 | 1SG | 3014 |
| ATOM | 3014 | N | GLY | 366 | 4.472 | 15.302 | 27.239 | 1.00 | 0.41 | 1SG | 3015 |
| ATOM | 3015 | CA | GLY | 366 | 4.022 | 14.242 | 28.097 | 1.00 | 0.41 | 1SG | 3016 |
| ATOM | 3016 | C | GLY | 366 | 4.244 | 14.718 | 29.488 | 1.00 | 0.41 | 1SG | 3017 |
| ATOM | 3017 | O | GLY | 366 | 5.160 | 14.240 | 30.154 | 1.00 | 0.41 | 1SG | 3018 |
| ATOM | 3018 | N | ALA | 367 | 3.396 | 15.668 | 29.938 | 1.00 | 0.29 | 1SG | 3019 |
| ATOM | 3019 | CA | ALA | 367 | 3.538 | 16.378 | 31.177 | 1.00 | 0.29 | 1SG | 3020 |
| ATOM | 3020 | CB | ALA | 367 | 2.253 | 17.107 | 31.615 | 1.00 | 0.29 | 1SG | 3021 |
| ATOM | 3021 | C | ALA | 367 | 4.005 | 15.478 | 32.279 | 1.00 | 0.29 | 1SG | 3022 |
| ATOM | 3022 | O | ALA | 367 | 5.029 | 15.804 | 32.871 | 1.00 | 0.29 | 1SG | 3023 |
| ATOM | 3023 | N | PRO | 368 | 3.388 | 14.374 | 32.609 | 1.00 | 0.42 | 1SG | 3024 |
| ATOM | 3024 | CA | PRO | 368 | 3.952 | 13.572 | 33.671 | 1.00 | 0.42 | 1SG | 3025 |
| ATOM | 3025 | CD | PRO | 368 | 1.939 | 14.261 | 32.566 | 1.00 | 0.42 | 1SG | 3026 |
| ATOM | 3026 | CB | PRO | 368 | 2.773 | 12.884 | 34.361 | 1.00 | 0.42 | 1SG | 3027 |
| ATOM | 3027 | CG | PRO | 368 | 1.622 | 12.979 | 33.347 | 1.00 | 0.42 | 1SG | 3028 |
| ATOM | 3028 | C | PRO | 368 | 4.982 | 12.600 | 33.148 | 1.00 | 0.42 | 1SG | 3029 |
| ATOM | 3029 | O | PRO | 368 | 4.930 | 12.318 | 31.955 | 1.00 | 0.42 | 1SG | 3030 |
| ATOM | 3030 | N | TYR | 369 | 5.930 | 12.084 | 33.983 | 1.00 | 0.49 | 1SG | 3031 |
| ATOM | 3031 | CA | TYR | 369 | 6.892 | 11.123 | 33.479 | 1.00 | 0.49 | 1SG | 3032 |
| ATOM | 3032 | CB | TYR | 369 | 7.903 | 11.757 | 32.505 | 1.00 | 0.49 | 1SG | 3033 |
| ATOM | 3033 | CG | TYR | 369 | 8.667 | 12.777 | 33.280 | 1.00 | 0.49 | 1SG | 3034 |
| ATOM | 3034 | CD1 | TYR | 369 | 8.131 | 14.026 | 33.489 | 1.00 | 0.49 | 1SG | 3035 |
| ATOM | 3035 | CD2 | TYR | 369 | 9.904 | 12.485 | 33.812 | 1.00 | 0.49 | 1SG | 3036 |
| ATOM | 3036 | CE1 | TYR | 369 | 8.818 | 14.973 | 34.210 | 1.00 | 0.49 | 1SG | 3037 |
| ATOM | 3037 | CE2 | TYR | 369 | 10.596 | 13.429 | 34.535 | 1.00 | 0.49 | 1SG | 3038 |
| ATOM | 3038 | CZ | TYR | 369 | 10.054 | 14.677 | 34.731 | 1.00 | 0.49 | 1SG | 3039 |
| ATOM | 3039 | OH | TYR | 369 | 10.760 | 15.649 | 35.471 | 1.00 | 0.49 | 1SG | 3040 |
| ATOM | 3040 | C | TYR | 369 | 7.692 | 10.532 | 34.612 | 1.00 | 0.49 | 1SG | 3041 |
| ATOM | 3041 | O | TYR | 369 | 8.140 | 11.249 | 35.505 | 1.00 | 0.49 | 1SG | 3042 |
| ATOM | 3042 | N | PRO | 370 | 7.852 | 9.230 | 34.641 | 1.00 | 0.42 | 1SG | 3043 |
| ATOM | 3043 | CA | PRO | 370 | 8.701 | 8.682 | 35.673 | 1.00 | 0.42 | 1SG | 3044 |
| ATOM | 3044 | CD | PRO | 370 | 6.692 | 8.386 | 34.408 | 1.00 | 0.42 | 1SG | 3045 |
| ATOM | 3045 | CB | PRO | 370 | 8.028 | 7.402 | 36.168 | 1.00 | 0.42 | 1SG | 3046 |
| ATOM | 3046 | CG | PRO | 370 | 7.048 | 7.035 | 35.045 | 1.00 | 0.42 | 1SG | 3047 |
| ATOM | 3047 | C | PRO | 370 | 10.125 | 8.478 | 35.241 | 1.00 | 0.42 | 1SG | 3048 |
| ATOM | 3048 | O | PRO | 370 | 10.337 | 7.962 | 34.144 | 1.00 | 0.42 | 1SG | 3049 |
| ATOM | 3049 | N | PRO | 371 | 11.088 | 8.863 | 36.042 | 1.00 | 0.43 | 1SG | 3050 |
| ATOM | 3050 | CA | PRO | 371 | 12.457 | 8.548 | 35.722 | 1.00 | 0.43 | 1SG | 3051 |
| ATOM | 3051 | CD | PRO | 371 | 10.985 | 10.158 | 36.689 | 1.00 | 0.43 | 1SG | 3052 |
| ATOM | 3052 | CB | PRO | 371 | 13.302 | 9.742 | 36.164 | 1.00 | 0.43 | 1SG | 3053 |
| ATOM | 3053 | CG | PRO | 371 | 12.408 | 10.485 | 37.166 | 1.00 | 0.43 | 1SG | 3054 |
| ATOM | 3054 | C | PRO | 371 | 12.793 | 7.293 | 36.450 | 1.00 | 0.43 | 1SG | 3055 |
| ATOM | 3055 | O | PRO | 371 | 12.215 | 7.082 | 37.513 | 1.00 | 0.43 | 1SG | 3056 |
| ATOM | 3056 | N | CYS | 372 | 13.758 | 6.488 | 35.954 | 1.00 | 0.54 | 1SG | 3057 |
| ATOM | 3057 | CA | CYS | 372 | 14.104 | 5.215 | 36.538 | 1.00 | 0.54 | 1SG | 3058 |
| ATOM | 3058 | CB | CYS | 372 | 15.038 | 5.363 | 37.757 | 1.00 | 0.54 | 1SG | 3059 |
| ATOM | 3059 | SG | CYS | 372 | 15.838 | 3.812 | 38.274 | 1.00 | 0.54 | 1SG | 3060 |
| ATOM | 3060 | C | CYS | 372 | 12.846 | 4.490 | 36.953 | 1.00 | 0.54 | 1SG | 3061 |
| ATOM | 3061 | O | CYS | 372 | 11.742 | 4.798 | 36.512 | 1.00 | 0.54 | 1SG | 3062 |
| ATOM | 3062 | N | SER | 373 | 12.964 | 3.446 | 37.778 | 1.00 | 0.48 | 1SG | 3063 |
| ATOM | 3063 | CA | SER | 373 | 11.779 | 2.749 | 38.179 | 1.00 | 0.48 | 1SG | 3064 |
| ATOM | 3064 | CB | SER | 373 | 12.071 | 1.571 | 39.123 | 1.00 | 0.48 | 1SG | 3065 |
| ATOM | 3065 | OG | SER | 373 | 10.862 | 0.919 | 39.483 | 1.00 | 0.48 | 1SG | 3066 |
| ATOM | 3066 | C | SER | 373 | 10.903 | 3.714 | 38.920 | 1.00 | 0.48 | 1SG | 3067 |
| ATOM | 3067 | O | SER | 373 | 9.679 | 3.652 | 38.830 | 1.00 | 0.48 | 1SG | 3068 |
| ATOM | 3068 | N | VAL | 374 | 11.530 | 4.639 | 39.661 | 1.00 | 0.35 | 1SG | 3069 |
| ATOM | 3069 | CA | VAL | 374 | 10.833 | 5.591 | 40.475 | 1.00 | 0.35 | 1SG | 3070 |
| ATOM | 3070 | CB | VAL | 374 | 11.752 | 6.634 | 41.045 | 1.00 | 0.35 | 1SG | 3071 |
| ATOM | 3071 | CG1 | VAL | 374 | 10.917 | 7.691 | 41.784 | 1.00 | 0.35 | 1SG | 3072 |
| ATOM | 3072 | CG2 | VAL | 374 | 12.799 | 5.934 | 41.926 | 1.00 | 0.35 | 1SG | 3073 |
| ATOM | 3073 | C | VAL | 374 | 9.793 | 6.298 | 39.672 | 1.00 | 0.35 | 1SG | 3074 |
| ATOM | 3074 | O | VAL | 374 | 9.940 | 6.526 | 38.473 | 1.00 | 0.35 | 1SG | 3075 |
| ATOM | 3075 | N | HIS | 375 | 8.673 | 6.645 | 40.331 | 1.00 | 0.40 | 1SG | 3076 |
| ATOM | 3076 | CA | HIS | 375 | 7.673 | 7.400 | 39.645 | 1.00 | 0.40 | 1SG | 3077 |
| ATOM | 3077 | ND1 | HIS | 375 | 4.669 | 8.862 | 39.490 | 1.00 | 0.40 | 1SG | 3078 |
| ATOM | 3078 | CG | HIS | 375 | 5.227 | 7.671 | 39.081 | 1.00 | 0.40 | 1SG | 3079 |
| ATOM | 3079 | CB | HIS | 375 | 6.238 | 6.900 | 39.876 | 1.00 | 0.40 | 1SG | 3080 |
| ATOM | 3080 | NE2 | HIS | 375 | 3.788 | 8.397 | 37.502 | 1.00 | 0.40 | 1SG | 3081 |
| ATOM | 3081 | CD2 | HIS | 375 | 4.678 | 7.402 | 37.864 | 1.00 | 0.40 | 1SG | 3082 |
| ATOM | 3082 | CE1 | HIS | 375 | 3.816 | 9.252 | 38.508 | 1.00 | 0.40 | 1SG | 3083 |
| ATOM | 3083 | C | HIS | 375 | 7.771 | 8.768 | 40.216 | 1.00 | 0.40 | 1SG | 3084 |
| ATOM | 3084 | O | HIS | 375 | 7.555 | 8.970 | 41.410 | 1.00 | 0.40 | 1SG | 3085 |
| ATOM | 3085 | N | VAL | 376 | 8.125 | 9.756 | 39.380 | 1.00 | 0.61 | 1SG | 3086 |
| ATOM | 3086 | CA | VAL | 376 | 8.232 | 11.059 | 39.946 | 1.00 | 0.61 | 1SG | 3087 |
| ATOM | 3087 | CB | VAL | 376 | 9.421 | 11.855 | 39.478 | 1.00 | 0.61 | 1SG | 3088 |
| ATOM | 3088 | CG1 | VAL | 376 | 10.706 | 11.066 | 39.775 | 1.00 | 0.61 | 1SG | 3089 |
| ATOM | 3089 | CG2 | VAL | 376 | 9.231 | 12.237 | 38.002 | 1.00 | 0.61 | 1SG | 3090 |
| ATOM | 3090 | C | VAL | 376 | 7.030 | 11.801 | 39.496 | 1.00 | 0.61 | 1SG | 3091 |
| ATOM | 3091 | O | VAL | 376 | 6.582 | 11.652 | 38.359 | 1.00 | 0.61 | 1SG | 3092 |
| ATOM | 3092 | N | ARG | 377 | 6.432 | 12.589 | 40.402 | 1.00 | 0.76 | 1SG | 3093 |
| ATOM | 3093 | CA | ARG | 377 | 5.380 | 13.406 | 39.907 | 1.00 | 0.76 | 1SG | 3094 |
| ATOM | 3094 | CB | ARG | 377 | 4.707 | 14.282 | 40.978 | 1.00 | 0.76 | 1SG | 3095 |
| ATOM | 3095 | CG | ARG | 377 | 3.844 | 13.475 | 41.948 | 1.00 | 0.76 | 1SG | 3096 |
| ATOM | 3096 | CD | ARG | 377 | 4.640 | 12.490 | 42.806 | 1.00 | 0.76 | 1SG | 3097 |
| ATOM | 3097 | NE | ARG | 377 | 3.664 | 11.728 | 43.636 | 1.00 | 0.76 | 1SG | 3098 |
| ATOM | 3098 | CZ | ARG | 377 | 3.920 | 10.426 | 43.956 | 1.00 | 0.76 | 1SG | 3099 |
| ATOM | 3099 | NH1 | ARG | 377 | 5.074 | 9.835 | 43.527 | 1.00 | 0.76 | 1SG | 3100 |
| ATOM | 3100 | NH2 | ARG | 377 | 3.022 | 9.713 | 44.697 | 1.00 | 0.76 | 1SG | 3101 |
| ATOM | 3101 | C | ARG | 377 | 6.109 | 14.284 | 38.971 | 1.00 | 0.76 | 1SG | 3102 |
| ATOM | 3102 | O | ARG | 377 | 7.300 | 14.514 | 39.192 | 1.00 | 0.76 | 1SG | 3103 |
| ATOM | 3103 | N | SER | 378 | 5.429 | 14.738 | 37.898 | 1.00 | 0.78 | 1SG | 3104 |
| ATOM | 3104 | CA | SER | 378 | 6.058 | 15.577 | 36.924 | 1.00 | 0.78 | 1SG | 3105 |
| ATOM | 3105 | CB | SER | 378 | 5.079 | 16.220 | 35.931 | 1.00 | 0.78 | 1SG | 3106 |
| ATOM | 3106 | OG | SER | 378 | 5.792 | 17.030 | 35.008 | 1.00 | 0.78 | 1SG | 3107 |
| ATOM | 3107 | C | SER | 378 | 6.707 | 16.669 | 37.682 | 1.00 | 0.78 | 1SG | 3108 |
| ATOM | 3108 | O | SER | 378 | 6.168 | 17.111 | 38.696 | 1.00 | 0.78 | 1SG | 3109 |
| ATOM | 3109 | N | VAL | 379 | 7.917 | 17.063 | 37.240 | 1.00 | 0.73 | 1SG | 3110 |
| ATOM | 3110 | CA | VAL | 379 | 8.625 | 18.096 | 37.923 | 1.00 | 0.73 | 1SG | 3111 |
| ATOM | 3111 | CB | VAL | 379 | 9.928 | 18.471 | 37.261 | 1.00 | 0.73 | 1SG | 3112 |
| ATOM | 3112 | CG1 | VAL | 379 | 9.672 | 18.899 | 35.810 | 1.00 | 0.73 | 1SG | 3113 |
| ATOM | 3113 | CG2 | VAL | 379 | 10.622 | 19.542 | 38.119 | 1.00 | 0.73 | 1SG | 3114 |
| ATOM | 3114 | C | VAL | 379 | 7.684 | 19.247 | 37.950 | 1.00 | 0.73 | 1SG | 3115 |
| ATOM | 3115 | O | VAL | 379 | 7.383 | 19.876 | 36.938 | 1.00 | 0.73 | 1SG | 3116 |
| ATOM | 3116 | N | CYS | 380 | 7.137 | 19.501 | 39.147 | 1.00 | 0.69 | 1SG | 3117 |
| ATOM | 3117 | CA | CYS | 380 | 6.120 | 20.491 | 39.228 | 1.00 | 0.69 | 1SG | 3118 |
| ATOM | 3118 | CB | CYS | 380 | 4.854 | 20.003 | 39.957 | 1.00 | 0.69 | 1SG | 3119 |
| ATOM | 3119 | SG | CYS | 380 | 3.567 | 21.282 | 40.058 | 1.00 | 0.69 | 1SG | 3120 |
| ATOM | 3120 | C | CYS | 380 | 6.658 | 21.651 | 39.981 | 1.00 | 0.69 | 1SG | 3121 |
| ATOM | 3121 | O | CYS | 380 | 7.578 | 21.530 | 40.787 | 1.00 | 0.69 | 1SG | 3122 |
| ATOM | 3122 | N | VAL | 381 | 6.101 | 22.833 | 39.677 | 1.00 | 0.68 | 1SG | 3123 |
| ATOM | 3123 | CA | VAL | 381 | 6.479 | 24.017 | 40.370 | 1.00 | 0.68 | 1SG | 3124 |
| ATOM | 3124 | CB | VAL | 381 | 6.211 | 25.257 | 39.551 | 1.00 | 0.68 | 1SG | 3125 |
| ATOM | 3125 | CG1 | VAL | 381 | 6.765 | 26.515 | 40.249 | 1.00 | 0.68 | 1SG | 3126 |
| ATOM | 3126 | CG2 | VAL | 381 | 6.775 | 25.031 | 38.143 | 1.00 | 0.68 | 1SG | 3127 |
| ATOM | 3127 | C | VAL | 381 | 5.555 | 23.985 | 41.546 | 1.00 | 0.68 | 1SG | 3128 |
| ATOM | 3128 | O | VAL | 381 | 5.058 | 22.918 | 41.907 | 1.00 | 0.68 | 1SG | 3129 |
| ATOM | 3129 | N | PHE | 382 | 5.310 | 25.130 | 42.201 | 1.00 | 0.60 | 1SG | 3130 |
| ATOM | 3130 | CA | PHE | 382 | 4.390 | 25.117 | 43.291 | 1.00 | 0.60 | 1SG | 3131 |
| ATOM | 3131 | CB | PHE | 382 | 4.586 | 26.316 | 44.244 | 1.00 | 0.60 | 1SG | 3132 |
| ATOM | 3132 | CG | PHE | 382 | 4.602 | 27.578 | 43.445 | 1.00 | 0.60 | 1SG | 3133 |
| ATOM | 3133 | CD1 | PHE | 382 | 5.772 | 27.982 | 42.843 | 1.00 | 0.60 | 1SG | 3134 |
| ATOM | 3134 | CD2 | PHE | 382 | 3.476 | 28.353 | 43.283 | 1.00 | 0.60 | 1SG | 3135 |
| ATOM | 3135 | CE1 | PHE | 382 | 5.821 | 29.137 | 42.098 | 1.00 | 0.60 | 1SG | 3136 |
| ATOM | 3136 | CE2 | PHE | 382 | 3.518 | 29.509 | 42.539 | 1.00 | 0.60 | 1SG | 3137 |
| ATOM | 3137 | CZ | PHE | 382 | 4.692 | 29.904 | 41.944 | 1.00 | 0.60 | 1SG | 3138 |
| ATOM | 3138 | C | PHE | 382 | 3.002 | 25.104 | 42.729 | 1.00 | 0.60 | 1SG | 3139 |
| ATOM | 3139 | O | PHE | 382 | 2.175 | 25.959 | 43.037 | 1.00 | 0.60 | 1SG | 3140 |
| ATOM | 3140 | N | GLY | 383 | 2.705 | 24.090 | 41.893 | 1.00 | 0.44 | 1SG | 3141 |
| ATOM | 3141 | CA | GLY | 383 | 1.394 | 23.956 | 41.328 | 1.00 | 0.44 | 1SG | 3142 |
| ATOM | 3142 | C | GLY | 383 | 1.078 | 25.180 | 40.524 | 1.00 | 0.44 | 1SG | 3143 |
| ATOM | 3143 | O | GLY | 383 | 0.033 | 25.799 | 40.717 | 1.00 | 0.44 | 1SG | 3144 |
| ATOM | 3144 | N | ALA | 384 | 1.976 | 25.562 | 39.594 | 1.00 | 0.44 | 1SG | 3145 |
| ATOM | 3145 | CA | ALA | 384 | 1.737 | 26.730 | 38.792 | 1.00 | 0.44 | 1SG | 3146 |
| ATOM | 3146 | CB | ALA | 384 | 2.942 | 27.684 | 38.730 | 1.00 | 0.44 | 1SG | 3147 |
| ATOM | 3147 | C | ALA | 384 | 1.453 | 26.284 | 37.391 | 1.00 | 0.44 | 1SG | 3148 |
| ATOM | 3148 | O | ALA | 384 | 0.704 | 25.334 | 37.169 | 1.00 | 0.44 | 1SG | 3149 |
| ATOM | 3149 | N | GLY | 385 | 2.050 | 26.981 | 36.400 | 1.00 | 0.47 | 1SG | 3150 |
| ATOM | 3150 | CA | GLY | 385 | 1.846 | 26.636 | 35.020 | 1.00 | 0.47 | 1SG | 3151 |
| ATOM | 3151 | C | GLY | 385 | 1.564 | 27.895 | 34.257 | 1.00 | 0.47 | 1SG | 3152 |
| ATOM | 3152 | O | GLY | 385 | 0.537 | 28.537 | 34.472 | 1.00 | 0.47 | 1SG | 3153 |
| ATOM | 3153 | N | ASP | 386 | 2.480 | 28.284 | 33.338 | 1.00 | 0.52 | 1SG | 3154 |
| ATOM | 3154 | CA | ASP | 386 | 2.263 | 29.492 | 32.591 | 1.00 | 0.52 | 1SG | 3155 |
| ATOM | 3155 | CB | ASP | 386 | 2.946 | 30.720 | 33.224 | 1.00 | 0.52 | 1SG | 3156 |
| ATOM | 3156 | CG | ASP | 386 | 2.412 | 31.987 | 32.568 | 1.00 | 0.52 | 1SG | 3157 |
| ATOM | 3157 | OD1 | ASP | 386 | 1.539 | 31.875 | 31.667 | 1.00 | 0.52 | 1SG | 3158 |
| ATOM | 3158 | OD2 | ASP | 386 | 2.873 | 33.090 | 32.965 | 1.00 | 0.52 | 1SG | 3159 |
| ATOM | 3159 | C | ASP | 386 | 2.824 | 29.332 | 31.209 | 1.00 | 0.52 | 1SG | 3160 |
| ATOM | 3160 | O | ASP | 386 | 3.960 | 28.900 | 31.023 | 1.00 | 0.52 | 1SG | 3161 |
| ATOM | 3161 | N | LEU | 387 | 2.040 | 29.763 | 30.202 | 1.00 | 0.61 | 1SG | 3162 |
| ATOM | 3162 | CA | LEU | 387 | 2.367 | 29.651 | 28.810 | 1.00 | 0.61 | 1SG | 3163 |
| ATOM | 3163 | CB | LEU | 387 | 1.251 | 30.195 | 27.895 | 1.00 | 0.61 | 1SG | 3164 |
| ATOM | 3164 | CG | LEU | 387 | 0.803 | 31.641 | 28.195 | 1.00 | 0.61 | 1SG | 3165 |
| ATOM | 3165 | CD2 | LEU | 387 | −0.548 | 31.949 | 27.531 | 1.00 | 0.61 | 1SG | 3166 |
| ATOM | 3166 | CD1 | LEU | 387 | 1.887 | 32.670 | 27.836 | 1.00 | 0.61 | 1SG | 3167 |
| ATOM | 3167 | C | LEU | 387 | 3.632 | 30.402 | 28.552 | 1.00 | 0.61 | 1SG | 3168 |
| ATOM | 3168 | O | LEU | 387 | 4.381 | 30.074 | 27.633 | 1.00 | 0.61 | 1SG | 3169 |
| ATOM | 3169 | N | ASN | 388 | 3.911 | 31.433 | 29.366 | 1.00 | 0.51 | 1SG | 3170 |
| ATOM | 3170 | CA | ASN | 388 | 5.087 | 32.227 | 29.168 | 1.00 | 0.51 | 1SG | 3171 |
| ATOM | 3171 | CB | ASN | 388 | 5.285 | 33.285 | 30.271 | 1.00 | 0.51 | 1SG | 3172 |
| ATOM | 3172 | CG | ASN | 388 | 6.455 | 34.181 | 29.884 | 1.00 | 0.51 | 1SG | 3173 |
| ATOM | 3173 | OD1 | ASN | 388 | 7.571 | 33.716 | 29.657 | 1.00 | 0.51 | 1SG | 3174 |
| ATOM | 3174 | ND2 | ASN | 388 | 6.193 | 35.513 | 29.803 | 1.00 | 0.51 | 1SG | 3175 |
| ATOM | 3175 | C | ASN | 388 | 6.268 | 31.309 | 29.197 | 1.00 | 0.51 | 1SG | 3176 |
| ATOM | 3176 | O | ASN | 388 | 7.230 | 31.506 | 28.455 | 1.00 | 0.51 | 1SG | 3177 |
| ATOM | 3177 | N | TRP | 389 | 6.221 | 30.271 | 30.052 | 1.00 | 0.63 | 1SG | 3178 |
| ATOM | 3178 | CA | TRP | 389 | 7.323 | 29.359 | 30.136 | 1.00 | 0.63 | 1SG | 3179 |
| ATOM | 3179 | CB | TRP | 389 | 7.062 | 28.185 | 31.094 | 1.00 | 0.63 | 1SG | 3180 |
| ATOM | 3180 | CG | TRP | 389 | 7.013 | 28.553 | 32.557 | 1.00 | 0.63 | 1SG | 3181 |
| ATOM | 3181 | CD2 | TRP | 389 | 7.964 | 28.094 | 33.529 | 1.00 | 0.63 | 1SG | 3182 |
| ATOM | 3182 | CD1 | TRP | 389 | 6.095 | 29.300 | 33.236 | 1.00 | 0.63 | 1SG | 3183 |
| ATOM | 3183 | NE1 | TRP | 389 | 6.420 | 29.341 | 34.571 | 1.00 | 0.63 | 1SG | 3184 |
| ATOM | 3184 | CE2 | TRP | 389 | 7.566 | 28.599 | 34.766 | 1.00 | 0.63 | 1SG | 3185 |
| ATOM | 3185 | CE3 | TRP | 389 | 9.075 | 27.309 | 33.401 | 1.00 | 0.63 | 1SG | 3186 |
| ATOM | 3186 | CZ2 | TRP | 389 | 8.276 | 28.325 | 35.899 | 1.00 | 0.63 | 1SG | 3187 |
| ATOM | 3187 | CZ3 | TRP | 389 | 9.791 | 27.039 | 34.545 | 1.00 | 0.63 | 1SG | 3188 |
| ATOM | 3188 | CH2 | TRP | 389 | 9.398 | 27.537 | 35.770 | 1.00 | 0.63 | 1SG | 3189 |
| ATOM | 3189 | C | TRP | 389 | 7.543 | 28.775 | 28.781 | 1.00 | 0.63 | 1SG | 3190 |
| ATOM | 3190 | O | TRP | 389 | 6.759 | 27.956 | 28.306 | 1.00 | 0.63 | 1SG | 3191 |
| ATOM | 3191 | N | LEU | 390 | 8.627 | 29.205 | 28.111 | 1.00 | 1.18 | 1SG | 3192 |
| ATOM | 3192 | CA | LEU | 390 | 8.943 | 28.672 | 26.821 | 1.00 | 1.18 | 1SG | 3193 |
| ATOM | 3193 | CB | LEU | 390 | 8.409 | 29.547 | 25.665 | 1.00 | 1.18 | 1SG | 3194 |
| ATOM | 3194 | CG | LEU | 390 | 8.710 | 29.036 | 24.240 | 1.00 | 1.18 | 1SG | 3195 |
| ATOM | 3195 | CD2 | LEU | 390 | 8.184 | 27.606 | 24.045 | 1.00 | 1.18 | 1SG | 3196 |
| ATOM | 3196 | CD1 | LEU | 390 | 10.193 | 29.184 | 23.864 | 1.00 | 1.18 | 1SG | 3197 |
| ATOM | 3197 | C | LEU | 390 | 10.433 | 28.638 | 26.749 | 1.00 | 1.18 | 1SG | 3198 |
| ATOM | 3198 | O | LEU | 390 | 11.101 | 29.494 | 27.318 | 1.00 | 1.18 | 1SG | 3199 |

TABLE 6
Core 2B human model

| REMARK Produced by MODELLER: 31 JUL. 2000 12:42:42 | | | | | | | | | | | 1 |
|---|---|---|---|---|---|---|---|---|---|---|---|
| REMARK MODELLER OBJECTIVE FUNCTION: 4509.6669 | | | | | | | | | | | |
| ATOM | 1 | N | MET | 1 | −75.085 | −53.625 | 35.229 | 1.00 | 0.63 | 1SG | 2 |
| ATOM | 2 | CA | MET | 1 | −74.578 | −54.884 | 35.818 | 1.00 | 0.63 | 1SG | 3 |
| ATOM | 3 | CB | MET | 1 | −74.153 | −55.856 | 34.704 | 1.00 | 0.63 | 1SG | 4 |
| ATOM | 4 | CG | MET | 1 | −73.816 | −57.261 | 35.206 | 1.00 | 0.63 | 1SG | 5 |
| ATOM | 5 | SD | MET | 1 | −75.252 | −58.217 | 35.780 | 1.00 | 0.63 | 1SG | 6 |
| ATOM | 6 | CE | MET | 1 | −75.987 | −58.415 | 34.131 | 1.00 | 0.63 | 1SG | 7 |
| ATOM | 7 | C | MET | 1 | −73.399 | −54.594 | 36.684 | 1.00 | 0.63 | 1SG | 8 |
| ATOM | 8 | O | MET | 1 | −73.488 | −53.816 | 37.633 | 1.00 | 0.63 | 1SG | 9 |
| ATOM | 9 | N | VAL | 2 | −72.252 | −55.224 | 36.377 | 1.00 | 0.72 | 1SG | 10 |
| ATOM | 10 | CA | VAL | 2 | −71.079 | −54.994 | 37.161 | 1.00 | 0.72 | 1SG | 11 |
| ATOM | 11 | CB | VAL | 2 | −69.931 | −55.891 | 36.787 | 1.00 | 0.72 | 1SG | 12 |
| ATOM | 12 | CG1 | VAL | 2 | −68.721 | −55.562 | 37.678 | 1.00 | 0.72 | 1SG | 13 |
| ATOM | 13 | CG2 | VAL | 2 | −70.404 | −57.350 | 36.894 | 1.00 | 0.72 | 1SG | 14 |
| ATOM | 14 | C | VAL | 2 | −70.668 | −53.581 | 36.919 | 1.00 | 0.72 | 1SG | 15 |
| ATOM | 15 | O | VAL | 2 | −71.036 | −52.979 | 35.911 | 1.00 | 0.72 | 1SG | 16 |
| ATOM | 16 | N | GLN | 3 | −69.904 | −53.004 | 37.865 | 1.00 | 1.11 | 1SG | 17 |
| ATOM | 17 | CA | GLN | 3 | −69.472 | −51.651 | 37.705 | 1.00 | 1.11 | 1SG | 18 |
| ATOM | 18 | CB | GLN | 3 | −68.883 | −51.042 | 38.995 | 1.00 | 1.11 | 1SG | 19 |
| ATOM | 19 | CG | GLN | 3 | −67.637 | −51.762 | 39.521 | 1.00 | 1.11 | 1SG | 20 |
| ATOM | 20 | CD | GLN | 3 | −66.419 | −51.251 | 38.764 | 1.00 | 1.11 | 1SG | 21 |
| ATOM | 21 | OE1 | GLN | 3 | −66.499 | −50.265 | 38.034 | 1.00 | 1.11 | 1SG | 22 |
| ATOM | 22 | NE2 | GLN | 3 | −65.258 | −51.935 | 38.945 | 1.00 | 1.11 | 1SG | 23 |
| ATOM | 23 | C | GLN | 3 | −68.427 | −51.637 | 36.641 | 1.00 | 1.11 | 1SG | 24 |
| ATOM | 24 | O | GLN | 3 | −67.764 | −52.642 | 36.393 | 1.00 | 1.11 | 1SG | 25 |
| ATOM | 25 | N | TRP | 4 | −68.275 | −50.485 | 35.964 | 1.00 | 1.38 | 1SG | 26 |
| ATOM | 26 | CA | TRP | 4 | −67.297 | −50.384 | 34.924 | 1.00 | 1.38 | 1SG | 27 |
| ATOM | 27 | CB | TRP | 4 | −67.850 | −49.783 | 33.621 | 1.00 | 1.38 | 1SG | 28 |
| ATOM | 28 | CG | TRP | 4 | −66.831 | −49.671 | 32.508 | 1.00 | 1.38 | 1SG | 29 |
| ATOM | 29 | CD2 | TRP | 4 | −66.553 | −50.720 | 31.568 | 1.00 | 1.38 | 1SG | 30 |
| ATOM | 30 | CD1 | TRP | 4 | −66.023 | −48.627 | 32.183 | 1.00 | 1.38 | 1SG | 31 |
| ATOM | 31 | NE1 | TRP | 4 | −65.248 | −48.958 | 31.092 | 1.00 | 1.38 | 1SG | 32 |
| ATOM | 32 | CE2 | TRP | 4 | −65.568 | −50.240 | 30.709 | 1.00 | 1.38 | 1SG | 33 |
| ATOM | 33 | CE3 | TRP | 4 | −67.072 | −51.971 | 31.434 | 1.00 | 1.38 | 1SG | 34 |
| ATOM | 34 | CZ2 | TRP | 4 | −65.083 | −51.018 | 29.688 | 1.00 | 1.38 | 1SG | 35 |
| ATOM | 35 | CZ3 | TRP | 4 | −66.586 | −52.753 | 30.410 | 1.00 | 1.38 | 1SG | 36 |
| ATOM | 36 | CH2 | TRP | 4 | −65.610 | −52.282 | 29.555 | 1.00 | 1.38 | 1SG | 37 |
| ATOM | 37 | C | TRP | 4 | −66.227 | −49.474 | 35.421 | 1.00 | 1.38 | 1SG | 38 |
| ATOM | 38 | O | TRP | 4 | −66.493 | −48.535 | 36.169 | 1.00 | 1.38 | 1SG | 39 |
| ATOM | 39 | N | LYS | 5 | −64.969 | −49.749 | 35.029 | 1.00 | 1.32 | 1SG | 40 |
| ATOM | 40 | CA | LYS | 5 | −63.896 | −48.914 | 35.467 | 1.00 | 1.32 | 1SG | 41 |
| ATOM | 41 | CB | LYS | 5 | −62.630 | −49.693 | 35.863 | 1.00 | 1.32 | 1SG | 42 |
| ATOM | 42 | CG | LYS | 5 | −61.483 | −48.801 | 36.338 | 1.00 | 1.32 | 1SG | 43 |
| ATOM | 43 | CD | LYS | 5 | −60.351 | −49.571 | 37.022 | 1.00 | 1.32 | 1SG | 44 |
| ATOM | 44 | CE | LYS | 5 | −59.506 | −50.402 | 36.055 | 1.00 | 1.32 | 1SG | 45 |
| ATOM | 45 | NZ | LYS | 5 | −58.437 | −51.107 | 36.798 | 1.00 | 1.32 | 1SG | 46 |
| ATOM | 46 | C | LYS | 5 | −63.530 | −48.028 | 34.324 | 1.00 | 1.32 | 1SG | 47 |
| ATOM | 47 | O | LYS | 5 | −63.463 | −48.469 | 33.177 | 1.00 | 1.32 | 1SG | 48 |
| ATOM | 48 | N | ARG | 6 | −63.298 | −46.736 | 34.614 | 1.00 | 1.17 | 1SG | 49 |
| ATOM | 49 | CA | ARG | 6 | −62.932 | −45.826 | 33.574 | 1.00 | 1.17 | 1SG | 50 |
| ATOM | 50 | CB | ARG | 6 | −63.813 | −44.565 | 33.522 | 1.00 | 1.17 | 1SG | 51 |
| ATOM | 51 | CG | ARG | 6 | −63.766 | −43.738 | 34.809 | 1.00 | 1.17 | 1SG | 52 |
| ATOM | 52 | CD | ARG | 6 | −64.638 | −42.482 | 34.765 | 1.00 | 1.17 | 1SG | 53 |
| ATOM | 53 | NE | ARG | 6 | −64.489 | −41.789 | 36.076 | 1.00 | 1.17 | 1SG | 54 |
| ATOM | 54 | CZ | ARG | 6 | −65.008 | −40.539 | 36.250 | 1.00 | 1.17 | 1SG | 55 |
| ATOM | 55 | NH1 | ARG | 6 | −65.661 | −39.921 | 35.223 | 1.00 | 1.17 | 1SG | 56 |
| ATOM | 56 | NH2 | ARG | 6 | −64.871 | −39.905 | 37.451 | 1.00 | 1.17 | 1SG | 57 |
| ATOM | 57 | C | ARG | 6 | −61.539 | −45.378 | 33.860 | 1.00 | 1.17 | 1SG | 58 |
| ATOM | 58 | O | ARG | 6 | −61.138 | −45.253 | 35.016 | 1.00 | 1.17 | 1SG | 59 |
| ATOM | 59 | N | LEU | 7 | −60.750 | −45.148 | 32.795 | 1.00 | 0.97 | 1SG | 60 |
| ATOM | 60 | CA | LEU | 7 | −59.406 | −44.699 | 32.990 | 1.00 | 0.97 | 1SG | 61 |
| ATOM | 61 | CB | LEU | 7 | −58.356 | −45.551 | 32.257 | 1.00 | 0.97 | 1SG | 62 |
| ATOM | 62 | CG | LEU | 7 | −58.284 | −47.004 | 32.759 | 1.00 | 0.97 | 1SG | 63 |
| ATOM | 63 | CD2 | LEU | 7 | −57.057 | −47.732 | 32.189 | 1.00 | 0.97 | 1SG | 64 |
| ATOM | 64 | CD1 | LEU | 7 | −59.600 | −47.754 | 32.493 | 1.00 | 0.97 | 1SG | 65 |
| ATOM | 65 | C | LEU | 7 | −59.328 | −43.321 | 32.429 | 1.00 | 0.97 | 1SG | 66 |
| ATOM | 66 | O | LEU | 7 | −59.926 | −43.024 | 31.398 | 1.00 | 0.97 | 1SG | 67 |
| ATOM | 67 | N | CYS | 8 | −58.597 | −42.426 | 33.120 | 1.00 | 0.94 | 1SG | 68 |
| ATOM | 68 | CA | CYS | 8 | −58.467 | −41.091 | 32.631 | 1.00 | 0.94 | 1SG | 69 |
| ATOM | 69 | CB | CYS | 8 | −58.924 | −40.017 | 33.635 | 1.00 | 0.94 | 1SG | 70 |
| ATOM | 70 | SG | CYS | 8 | −58.750 | −38.331 | 32.979 | 1.00 | 0.94 | 1SG | 71 |
| ATOM | 71 | C | CYS | 8 | −57.018 | −40.858 | 32.365 | 1.00 | 0.94 | 1SG | 72 |
| ATOM | 72 | O | CYS | 8 | −56.159 | −41.319 | 33.110 | 1.00 | 0.94 | 1SG | 73 |
| ATOM | 73 | N | GLN | 9 | −56.716 | −40.148 | 31.263 | 1.00 | 1.18 | 1SG | 74 |
| ATOM | 74 | CA | GLN | 9 | −55.347 | −39.865 | 30.953 | 1.00 | 1.18 | 1SG | 75 |
| ATOM | 75 | CB | GLN | 9 | −54.926 | −40.342 | 29.552 | 1.00 | 1.18 | 1SG | 76 |
| ATOM | 76 | CG | GLN | 9 | −53.463 | −40.059 | 29.199 | 1.00 | 1.18 | 1SG | 77 |
| ATOM | 77 | CD | GLN | 9 | −53.349 | −38.619 | 28.716 | 1.00 | 1.18 | 1SG | 78 |
| ATOM | 78 | OE1 | GLN | 9 | −52.275 | −38.023 | 28.749 | 1.00 | 1.18 | 1SG | 79 |
| ATOM | 79 | NE2 | GLN | 9 | −54.485 | −38.038 | 28.247 | 1.00 | 1.18 | 1SG | 80 |
| ATOM | 80 | C | GLN | 9 | −55.195 | −38.384 | 31.004 | 1.00 | 1.18 | 1SG | 81 |
| ATOM | 81 | O | GLN | 9 | −56.066 | −37.644 | 30.552 | 1.00 | 1.18 | 1SG | 82 |
| ATOM | 82 | N | LEU | 10 | −54.078 | −37.908 | 31.582 | 1.00 | 1.32 | 1SG | 83 |
| ATOM | 83 | CA | LEU | 10 | −53.864 | −36.499 | 31.646 | 1.00 | 1.32 | 1SG | 84 |
| ATOM | 84 | CB | LEU | 10 | −53.662 | −35.971 | 33.079 | 1.00 | 1.32 | 1SG | 85 |
| ATOM | 85 | CG | LEU | 10 | −53.434 | −34.451 | 33.148 | 1.00 | 1.32 | 1SG | 86 |
| ATOM | 86 | CD2 | LEU | 10 | −52.978 | −34.016 | 34.551 | 1.00 | 1.32 | 1SG | 87 |
| ATOM | 87 | CD1 | LEU | 10 | −54.665 | −33.675 | 32.658 | 1.00 | 1.32 | 1SG | 88 |
| ATOM | 88 | C | LEU | 10 | −52.615 | −36.204 | 30.890 | 1.00 | 1.32 | 1SG | 89 |
| ATOM | 89 | O | LEU | 10 | −51.639 | −36.949 | 30.970 | 1.00 | 1.32 | 1SG | 90 |
| ATOM | 90 | N | HIS | 11 | −52.628 | −35.110 | 30.109 | 1.00 | 1.17 | 1SG | 91 |
| ATOM | 91 | CA | HIS | 11 | −51.459 | −34.753 | 29.363 | 1.00 | 1.17 | 1SG | 92 |
| ATOM | 92 | ND1 | HIS | 11 | −52.477 | −32.416 | 27.185 | 1.00 | 1.17 | 1SG | 93 |
| ATOM | 93 | CG | HIS | 11 | −52.722 | −33.753 | 27.410 | 1.00 | 1.17 | 1SG | 94 |
| ATOM | 94 | CB | HIS | 11 | −51.676 | −34.738 | 27.843 | 1.00 | 1.17 | 1SG | 95 |
| ATOM | 95 | NE2 | HIS | 11 | −54.642 | −32.740 | 26.793 | 1.00 | 1.17 | 1SG | 96 |
| ATOM | 96 | CD2 | HIS | 11 | −54.050 | −33.933 | 27.167 | 1.00 | 1.17 | 1SG | 97 |
| ATOM | 97 | CE1 | HIS | 11 | −53.659 | −31.859 | 26.819 | 1.00 | 1.17 | 1SG | 98 |
| ATOM | 98 | C | HIS | 11 | −51.090 | −33.367 | 29.771 | 1.00 | 1.17 | 1SG | 99 |
| ATOM | 99 | O | HIS | 11 | −51.956 | −32.541 | 30.050 | 1.00 | 1.17 | 1SG | 100 |
| ATOM | 100 | N | TYR | 12 | −49.776 | −33.086 | 29.839 | 1.00 | 1.07 | 1SG | 101 |
| ATOM | 101 | CA | TYR | 12 | −49.353 | −31.767 | 30.205 | 1.00 | 1.07 | 1SG | 102 |
| ATOM | 102 | CB | TYR | 12 | −48.413 | −31.736 | 31.421 | 1.00 | 1.07 | 1SG | 103 |
| ATOM | 103 | CG | TYR | 12 | −48.065 | −30.310 | 31.681 | 1.00 | 1.07 | 1SG | 104 |
| ATOM | 104 | CD1 | TYR | 12 | −48.914 | −29.500 | 32.397 | 1.00 | 1.07 | 1SG | 105 |
| ATOM | 105 | CD2 | TYR | 12 | −46.883 | −29.784 | 31.208 | 1.00 | 1.07 | 1SG | 106 |
| ATOM | 106 | CE1 | TYR | 12 | −48.593 | −28.185 | 32.639 | 1.00 | 1.07 | 1SG | 107 |
| ATOM | 107 | CE2 | TYR | 12 | −46.557 | −28.469 | 31.446 | 1.00 | 1.07 | 1SG | 108 |
| ATOM | 108 | CZ | TYR | 12 | −47.412 | −27.669 | 32.163 | 1.00 | 1.07 | 1SG | 109 |
| ATOM | 109 | OH | TYR | 12 | −47.080 | −26.319 | 32.407 | 1.00 | 1.07 | 1SG | 110 |
| ATOM | 110 | C | TYR | 12 | −48.597 | −31.214 | 29.047 | 1.00 | 1.07 | 1SG | 111 |
| ATOM | 111 | O | TYR | 12 | −47.765 | −31.898 | 28.449 | 1.00 | 1.07 | 1SG | 112 |
| ATOM | 112 | N | LEU | 13 | −48.884 | −29.953 | 28.683 | 1.00 | 1.16 | 1SG | 113 |
| ATOM | 113 | CA | LEU | 13 | −48.187 | −29.347 | 27.589 | 1.00 | 1.16 | 1SG | 114 |
| ATOM | 114 | CB | LEU | 13 | −49.044 | −29.191 | 26.320 | 1.00 | 1.16 | 1SG | 115 |
| ATOM | 115 | CG | LEU | 13 | −48.291 | −28.540 | 25.145 | 1.00 | 1.16 | 1SG | 116 |
| ATOM | 116 | CD2 | LEU | 13 | −49.251 | −28.166 | 24.005 | 1.00 | 1.16 | 1SG | 117 |
| ATOM | 117 | CD1 | LEU | 13 | −47.123 | −29.417 | 24.678 | 1.00 | 1.16 | 1SG | 118 |
| ATOM | 118 | C | LEU | 13 | −47.791 | −27.979 | 28.025 | 1.00 | 1.16 | 1SG | 119 |
| ATOM | 119 | O | LEU | 13 | −48.356 | −27.435 | 28.974 | 1.00 | 1.16 | 1SG | 120 |
| ATOM | 120 | N | TRP | 14 | −46.782 | −27.398 | 27.355 | 1.00 | 1.09 | 1SG | 121 |
| ATOM | 121 | CA | TRP | 14 | −46.369 | −26.073 | 27.706 | 1.00 | 1.09 | 1SG | 122 |
| ATOM | 122 | CB | TRP | 14 | −45.227 | −26.024 | 28.737 | 1.00 | 1.09 | 1SG | 123 |
| ATOM | 123 | CG | TRP | 14 | −45.155 | −24.766 | 29.583 | 1.00 | 1.09 | 1SG | 124 |
| ATOM | 124 | CD2 | TRP | 14 | −45.323 | −23.405 | 29.139 | 1.00 | 1.09 | 1SG | 125 |
| ATOM | 125 | CD1 | TRP | 14 | −44.990 | −24.704 | 30.937 | 1.00 | 1.09 | 1SG | 126 |
| ATOM | 126 | NE1 | TRP | 14 | −45.022 | −23.398 | 31.361 | 1.00 | 1.09 | 1SG | 127 |
| ATOM | 127 | CE2 | TRP | 14 | −45.233 | −22.588 | 30.267 | 1.00 | 1.09 | 1SG | 128 |
| ATOM | 128 | CE3 | TRP | 14 | −45.550 | −22.873 | 27.901 | 1.00 | 1.09 | 1SG | 129 |
| ATOM | 129 | CZ2 | TRP | 14 | −45.363 | −21.230 | 30.172 | 1.00 | 1.09 | 1SG | 130 |
| ATOM | 130 | CZ3 | TRP | 14 | −45.666 | −21.506 | 27.805 | 1.00 | 1.09 | 1SG | 131 |
| ATOM | 131 | CH2 | TRP | 14 | −45.574 | −20.698 | 28.919 | 1.00 | 1.09 | 1SG | 132 |
| ATOM | 132 | C | TRP | 14 | −45.808 | −25.528 | 26.433 | 1.00 | 1.09 | 1SG | 133 |
| ATOM | 133 | O | TRP | 14 | −46.484 | −25.504 | 25.406 | 1.00 | 1.09 | 1SG | 134 |
| ATOM | 134 | N | ALA | 15 | −44.543 | −25.070 | 26.468 | 1.00 | 1.00 | 1SG | 135 |
| ATOM | 135 | CA | ALA | 15 | −43.937 | −24.551 | 25.280 | 1.00 | 1.00 | 1SG | 136 |
| ATOM | 136 | CB | ALA | 15 | −43.448 | −23.096 | 25.423 | 1.00 | 1.00 | 1SG | 137 |
| ATOM | 137 | C | ALA | 15 | −42.737 | −25.391 | 25.008 | 1.00 | 1.00 | 1SG | 138 |
| ATOM | 138 | O | ALA | 15 | −42.055 | −25.842 | 25.925 | 1.00 | 1.00 | 1SG | 139 |
| ATOM | 139 | N | LEU | 16 | −42.459 | −25.647 | 23.715 | 1.00 | 0.90 | 1SG | 140 |
| ATOM | 140 | CA | LEU | 16 | −41.322 | −26.450 | 23.383 | 1.00 | 0.90 | 1SG | 141 |
| ATOM | 141 | CB | LEU | 16 | −41.701 | −27.900 | 23.026 | 1.00 | 0.90 | 1SG | 142 |
| ATOM | 142 | CG | LEU | 16 | −40.528 | −28.838 | 22.661 | 1.00 | 0.90 | 1SG | 143 |
| ATOM | 143 | CD2 | LEU | 16 | −39.484 | −28.866 | 23.783 | 1.00 | 0.90 | 1SG | 144 |
| ATOM | 144 | CD1 | LEU | 16 | −39.921 | −28.512 | 21.283 | 1.00 | 0.90 | 1SG | 145 |
| ATOM | 145 | C | LEU | 16 | −40.658 | −25.815 | 22.212 | 1.00 | 0.90 | 1SG | 146 |
| ATOM | 146 | O | LEU | 16 | −41.319 | −25.236 | 21.352 | 1.00 | 0.90 | 1SG | 147 |
| ATOM | 147 | N | GLY | 17 | −39.314 | −25.898 | 22.161 | 1.00 | 0.57 | 1SG | 148 |
| ATOM | 148 | CA | GLY | 17 | −38.593 | −25.370 | 21.044 | 1.00 | 0.57 | 1SG | 149 |
| ATOM | 149 | C | GLY | 17 | −38.586 | −23.882 | 21.143 | 1.00 | 0.57 | 1SG | 150 |
| ATOM | 150 | O | GLY | 17 | −38.436 | −23.188 | 20.138 | 1.00 | 0.57 | 1SG | 151 |
| ATOM | 151 | N | CYS | 18 | −38.748 | −23.342 | 22.364 | 1.00 | 0.56 | 1SG | 152 |
| ATOM | 152 | CA | CYS | 18 | −38.739 | −21.917 | 22.486 | 1.00 | 0.56 | 1SG | 153 |
| ATOM | 153 | CB | CYS | 18 | −39.560 | −21.389 | 23.677 | 1.00 | 0.56 | 1SG | 154 |
| ATOM | 154 | SG | CYS | 18 | −38.998 | −22.032 | 25.284 | 1.00 | 0.56 | 1SG | 155 |
| ATOM | 155 | C | CYS | 18 | −37.318 | −21.491 | 22.663 | 1.00 | 0.56 | 1SG | 156 |
| ATOM | 156 | O | CYS | 18 | −36.662 | −21.851 | 23.636 | 1.00 | 0.56 | 1SG | 157 |
| ATOM | 157 | N | TYR | 19 | −36.798 | −20.726 | 21.689 | 1.00 | 0.93 | 1SG | 158 |
| ATOM | 158 | CA | TYR | 19 | −35.447 | −20.259 | 21.775 | 1.00 | 0.93 | 1SG | 159 |
| ATOM | 159 | CB | TYR | 19 | −34.492 | −20.969 | 20.795 | 1.00 | 0.93 | 1SG | 160 |
| ATOM | 160 | CG | TYR | 19 | −33.127 | −20.399 | 20.982 | 1.00 | 0.93 | 1SG | 161 |
| ATOM | 161 | CD1 | TYR | 19 | −32.751 | −19.246 | 20.328 | 1.00 | 0.93 | 1SG | 162 |
| ATOM | 162 | CD2 | TYR | 19 | −32.219 | −21.019 | 21.807 | 1.00 | 0.93 | 1SG | 163 |
| ATOM | 163 | CE1 | TYR | 19 | −31.492 | −18.721 | 20.500 | 1.00 | 0.93 | 1SG | 164 |
| ATOM | 164 | CE2 | TYR | 19 | −30.958 | −20.500 | 21.982 | 1.00 | 0.93 | 1SG | 165 |
| ATOM | 165 | CZ | TYR | 19 | −30.594 | −19.348 | 21.328 | 1.00 | 0.93 | 1SG | 166 |
| ATOM | 166 | OH | TYR | 19 | −29.302 | −18.808 | 21.503 | 1.00 | 0.93 | 1SG | 167 |
| ATOM | 167 | C | TYR | 19 | −35.453 | −18.813 | 21.428 | 1.00 | 0.93 | 1SG | 168 |
| ATOM | 168 | O | TYR | 19 | −36.227 | −18.367 | 20.586 | 1.00 | 0.93 | 1SG | 169 |
| ATOM | 169 | N | MET | 20 | −34.589 | −18.029 | 22.101 | 1.00 | 1.20 | 1SG | 170 |
| ATOM | 170 | CA | MET | 20 | −34.511 | −16.632 | 21.797 | 1.00 | 1.20 | 1SG | 171 |
| ATOM | 171 | CB | MET | 20 | −34.882 | −15.722 | 22.985 | 1.00 | 1.20 | 1SG | 172 |
| ATOM | 172 | CG | MET | 20 | −34.804 | −14.229 | 22.655 | 1.00 | 1.20 | 1SG | 173 |
| ATOM | 173 | SD | MET | 20 | −35.234 | −13.126 | 24.034 | 1.00 | 1.20 | 1SG | 174 |
| ATOM | 174 | CE | MET | 20 | −34.972 | −11.593 | 23.095 | 1.00 | 1.20 | 1SG | 175 |
| ATOM | 175 | C | MET | 20 | −33.091 | −16.338 | 21.459 | 1.00 | 1.20 | 1SG | 176 |
| ATOM | 176 | O | MET | 20 | −32.182 | −16.646 | 22.225 | 1.00 | 1.20 | 1SG | 177 |
| ATOM | 177 | N | LEU | 21 | −32.857 | −15.748 | 20.274 | 1.00 | 1.29 | 1SG | 178 |
| ATOM | 178 | CA | LEU | 21 | −31.516 | −15.410 | 19.901 | 1.00 | 1.29 | 1SG | 179 |
| ATOM | 179 | CB | LEU | 21 | −30.978 | −16.218 | 18.709 | 1.00 | 1.29 | 1SG | 180 |
| ATOM | 180 | CG | LEU | 21 | −29.529 | −15.853 | 18.328 | 1.00 | 1.29 | 1SG | 181 |
| ATOM | 181 | CD2 | LEU | 21 | −29.130 | −16.500 | 16.988 | 1.00 | 1.29 | 1SG | 182 |
| ATOM | 182 | CD1 | LEU | 21 | −28.550 | −16.179 | 19.460 | 1.00 | 1.29 | 1SG | 183 |
| ATOM | 183 | C | LEU | 21 | −31.525 | −13.973 | 19.511 | 1.00 | 1.29 | 1SG | 184 |
| ATOM | 184 | O | LEU | 21 | −32.476 | −13.500 | 18.893 | 1.00 | 1.29 | 1SG | 185 |
| ATOM | 185 | N | LEU | 22 | −30.465 | −13.234 | 19.887 | 1.00 | 1.05 | 1SG | 186 |
| ATOM | 186 | CA | LEU | 22 | −30.418 | −11.848 | 19.539 | 1.00 | 1.05 | 1SG | 187 |
| ATOM | 187 | CB | LEU | 22 | −30.222 | −10.918 | 20.749 | 1.00 | 1.05 | 1SG | 188 |
| ATOM | 188 | CG | LEU | 22 | −31.386 | −10.962 | 21.755 | 1.00 | 1.05 | 1SG | 189 |
| ATOM | 189 | CD2 | LEU | 22 | −31.541 | −12.361 | 22.373 | 1.00 | 1.05 | 1SG | 190 |
| ATOM | 190 | CD1 | LEU | 22 | −32.687 | −10.436 | 21.129 | 1.00 | 1.05 | 1SG | 191 |
| ATOM | 191 | C | LEU | 22 | −29.243 | −11.655 | 18.642 | 1.00 | 1.05 | 1SG | 192 |
| ATOM | 192 | O | LEU | 22 | −28.174 | −12.218 | 18.872 | 1.00 | 1.05 | 1SG | 193 |
| ATOM | 193 | N | ALA | 23 | −29.423 | −10.859 | 17.572 | 1.00 | 0.74 | 1SG | 194 |
| ATOM | 194 | CA | ALA | 23 | −28.332 | −10.608 | 16.681 | 1.00 | 0.74 | 1SG | 195 |
| ATOM | 195 | CB | ALA | 23 | −28.563 | −11.142 | 15.257 | 1.00 | 0.74 | 1SG | 196 |
| ATOM | 196 | C | ALA | 23 | −28.185 | −9.127 | 16.578 | 1.00 | 0.74 | 1SG | 197 |
| ATOM | 197 | O | ALA | 23 | −29.166 | −8.405 | 16.411 | 1.00 | 0.74 | 1SG | 198 |
| ATOM | 198 | N | THR | 24 | −26.939 | −8.634 | 15.701 | 1.00 | 0.71 | 1SG | 199 |
| ATOM | 199 | CA | THR | 24 | −26.714 | −7.226 | 16.587 | 1.00 | 0.71 | 1SG | 200 |
| ATOM | 200 | CB | THR | 24 | −26.281 | −6.579 | 17.869 | 1.00 | 0.71 | 1SG | 201 |
| ATOM | 201 | OG1 | THR | 24 | −26.285 | −5.166 | 17.732 | 1.00 | 0.71 | 1SG | 202 |
| ATOM | 202 | CG2 | THR | 24 | −24.869 | −7.077 | 18.221 | 1.00 | 0.71 | 1SG | 203 |
| ATOM | 203 | C | THR | 24 | −25.609 | −7.042 | 15.602 | 1.00 | 0.71 | 1SG | 204 |
| ATOM | 204 | O | THR | 24 | −24.696 | −7.862 | 15.521 | 1.00 | 0.71 | 1SG | 205 |
| ATOM | 205 | N | VAL | 25 | −25.673 | −5.959 | 14.806 | 1.00 | 0.70 | 1SG | 206 |
| ATOM | 206 | CA | VAL | 25 | −24.637 | −5.736 | 13.845 | 1.00 | 0.70 | 1SG | 207 |
| ATOM | 207 | CB | VAL | 25 | −25.097 | −5.878 | 12.425 | 1.00 | 0.70 | 1SG | 208 |
| ATOM | 208 | CG1 | VAL | 25 | −26.159 | −4.801 | 12.146 | 1.00 | 0.70 | 1SG | 209 |
| ATOM | 209 | CG2 | VAL | 25 | −23.867 | −5.790 | 11.506 | 1.00 | 0.70 | 1SG | 210 |
| ATOM | 210 | C | VAL | 25 | −24.152 | −4.335 | 14.014 | 1.00 | 0.70 | 1SG | 211 |
| ATOM | 211 | O | VAL | 25 | −24.895 | −3.455 | 14.444 | 1.00 | 0.70 | 1SG | 212 |
| ATOM | 212 | N | ALA | 26 | −22.864 | −4.104 | 13.698 | 1.00 | 0.85 | 1SG | 213 |
| ATOM | 213 | CA | ALA | 26 | −22.319 | −2.784 | 13.795 | 1.00 | 0.85 | 1SG | 214 |
| ATOM | 214 | CB | ALA | 26 | −21.684 | −2.478 | 15.163 | 1.00 | 0.85 | 1SG | 215 |
| ATOM | 215 | C | ALA | 26 | −21.234 | −2.695 | 12.777 | 1.00 | 0.85 | 1SG | 216 |
| ATOM | 216 | O | ALA | 26 | −20.801 | −3.706 | 12.229 | 1.00 | 0.85 | 1SG | 217 |
| ATOM | 217 | N | LEU | 27 | −20.782 | −1.460 | 12.481 | 1.00 | 1.04 | 1SG | 218 |
| ATOM | 218 | CA | LEU | 27 | −19.717 | −1.314 | 11.532 | 1.00 | 1.04 | 1SG | 219 |
| ATOM | 219 | CB | LEU | 27 | −20.075 | −0.448 | 10.317 | 1.00 | 1.04 | 1SG | 220 |
| ATOM | 220 | CG | LEU | 27 | −18.910 | −0.298 | 9.319 | 1.00 | 1.04 | 1SG | 221 |
| ATOM | 221 | CD2 | LEU | 27 | −19.208 | 0.778 | 8.264 | 1.00 | 1.04 | 1SG | 222 |
| ATOM | 222 | CD1 | LEU | 27 | −18.525 | −1.656 | 8.702 | 1.00 | 1.04 | 1SG | 223 |
| ATOM | 223 | C | LEU | 27 | −18.605 | −0.623 | 12.249 | 1.00 | 1.04 | 1SG | 224 |
| ATOM | 224 | O | LEU | 27 | −18.833 | 0.289 | 13.042 | 1.00 | 1.04 | 1SG | 225 |
| ATOM | 225 | N | LYS | 28 | −17.359 | −1.065 | 12.004 | 1.00 | 0.95 | 1SG | 226 |
| ATOM | 226 | CA | LYS | 28 | −16.255 | −0.434 | 12.655 | 1.00 | 0.95 | 1SG | 227 |
| ATOM | 227 | CB | LYS | 28 | −15.863 | −1.144 | 13.964 | 1.00 | 0.95 | 1SG | 228 |
| ATOM | 228 | CG | LYS | 28 | −16.993 | −1.114 | 14.998 | 1.00 | 0.95 | 1SG | 229 |
| ATOM | 229 | CD | LYS | 28 | −16.834 | −2.113 | 16.147 | 1.00 | 0.95 | 1SG | 230 |
| ATOM | 230 | CE | LYS | 28 | −17.991 | −2.072 | 17.150 | 1.00 | 0.95 | 1SG | 231 |
| ATOM | 231 | NZ | LYS | 28 | −17.785 | −3.079 | 18.215 | 1.00 | 0.95 | 1SG | 232 |
| ATOM | 232 | C | LYS | 28 | −15.103 | −0.505 | 11.710 | 1.00 | 0.95 | 1SG | 233 |
| ATOM | 233 | O | LYS | 28 | −14.434 | −1.533 | 11.606 | 1.00 | 0.95 | 1SG | 234 |
| ATOM | 234 | N | LEU | 29 | −14.850 | 0.596 | 10.978 | 1.00 | 0.89 | 1SG | 235 |
| ATOM | 235 | CA | LEU | 29 | −13.764 | 0.565 | 10.053 | 1.00 | 0.89 | 1SG | 236 |
| ATOM | 236 | CB | LEU | 29 | −14.210 | 0.594 | 8.583 | 1.00 | 0.89 | 1SG | 237 |
| ATOM | 237 | CG | LEU | 29 | −15.049 | −0.636 | 8.181 | 1.00 | 0.89 | 1SG | 238 |
| ATOM | 238 | CD2 | LEU | 29 | −14.335 | −1.944 | 8.539 | 1.00 | 0.89 | 1SG | 239 |
| ATOM | 239 | CD1 | LEU | 29 | −15.474 | −0.568 | 6.709 | 1.00 | 0.89 | 1SG | 240 |
| ATOM | 240 | C | LEU | 29 | −12.913 | 1.769 | 10.284 | 1.00 | 0.89 | 1SG | 241 |
| ATOM | 241 | O | LEU | 29 | −13.416 | 2.880 | 10.454 | 1.00 | 0.89 | 1SG | 242 |
| ATOM | 242 | N | SER | 30 | −11.584 | 1.554 | 10.320 | 1.00 | 0.79 | 1SG | 243 |
| ATOM | 243 | CA | SER | 30 | −10.646 | 2.624 | 10.477 | 1.00 | 0.79 | 1SG | 244 |
| ATOM | 244 | CB | SER | 30 | −9.707 | 2.449 | 11.682 | 1.00 | 0.79 | 1SG | 245 |
| ATOM | 245 | OG | SER | 30 | −8.713 | 3.462 | 11.682 | 1.00 | 0.79 | 1SG | 246 |
| ATOM | 246 | C | SER | 30 | −9.782 | 2.582 | 9.260 | 1.00 | 0.79 | 1SG | 247 |
| ATOM | 247 | O | SER | 30 | −9.157 | 1.562 | 8.978 | 1.00 | 0.79 | 1SG | 248 |
| ATOM | 248 | N | PHE | 31 | −9.718 | 3.688 | 8.494 | 1.00 | 0.71 | 1SG | 249 |
| ATOM | 249 | CA | PHE | 31 | −8.899 | 3.639 | 7.319 | 1.00 | 0.71 | 1SG | 250 |
| ATOM | 250 | CB | PHE | 31 | −9.682 | 3.737 | 5.995 | 1.00 | 0.71 | 1SG | 251 |
| ATOM | 251 | CG | PHE | 31 | −10.354 | 5.061 | 5.896 | 1.00 | 0.71 | 1SG | 252 |
| ATOM | 252 | CD1 | PHE | 31 | −9.643 | 6.165 | 5.494 | 1.00 | 0.71 | 1SG | 253 |
| ATOM | 253 | CD2 | PHE | 31 | −11.693 | 5.196 | 6.184 | 1.00 | 0.71 | 1SG | 254 |
| ATOM | 254 | CE1 | PHE | 31 | −10.246 | 7.395 | 5.386 | 1.00 | 0.71 | 1SG | 255 |
| ATOM | 255 | CE2 | PHE | 31 | −12.307 | 6.422 | 6.080 | 1.00 | 0.71 | 1SG | 256 |
| ATOM | 256 | CZ | PHE | 31 | −11.583 | 7.522 | 5.681 | 1.00 | 0.71 | 1SG | 257 |
| ATOM | 257 | C | PHE | 31 | −7.887 | 4.745 | 7.388 | 1.00 | 0.71 | 1SG | 258 |
| ATOM | 258 | O | PHE | 31 | −7.761 | 5.412 | 8.414 | 1.00 | 0.71 | 1SG | 259 |
| ATOM | 259 | N | ARG | 32 | −7.123 | 4.947 | 6.288 | 1.00 | 0.71 | 1SG | 260 |
| ATOM | 260 | CA | ARG | 32 | −6.048 | 5.907 | 6.285 | 1.00 | 0.71 | 1SG | 261 |
| ATOM | 261 | CB | ARG | 32 | −4.688 | 5.258 | 5.993 | 1.00 | 0.71 | 1SG | 262 |
| ATOM | 262 | CG | ARG | 32 | −4.344 | 4.135 | 6.973 | 1.00 | 0.71 | 1SG | 263 |
| ATOM | 263 | CD | ARG | 32 | −3.068 | 3.383 | 6.599 | 1.00 | 0.71 | 1SG | 264 |
| ATOM | 264 | NE | ARG | 32 | −1.921 | 4.246 | 6.989 | 1.00 | 0.71 | 1SG | 265 |
| ATOM | 265 | CZ | ARG | 32 | −0.753 | 4.186 | 6.286 | 1.00 | 0.71 | 1SG | 266 |
| ATOM | 266 | NH1 | ARG | 32 | −0.642 | 3.357 | 5.207 | 1.00 | 0.71 | 1SG | 267 |
| ATOM | 267 | NH2 | ARG | 32 | 0.303 | 4.963 | 6.662 | 1.00 | 0.71 | 1SG | 268 |
| ATOM | 268 | C | ARG | 32 | −6.273 | 6.932 | 5.202 | 1.00 | 0.71 | 1SG | 269 |
| ATOM | 269 | O | ARG | 32 | −7.209 | 6.810 | 4.418 | 1.00 | 0.71 | 1SG | 270 |
| ATOM | 270 | N | LEU | 33 | −5.404 | 7.979 | 5.150 | 1.00 | 0.75 | 1SG | 271 |
| ATOM | 271 | CA | LEU | 33 | −5.494 | 9.082 | 4.210 | 1.00 | 0.75 | 1SG | 272 |
| ATOM | 272 | CB | LEU | 33 | −5.952 | 10.380 | 4.893 | 1.00 | 0.75 | 1SG | 273 |
| ATOM | 273 | CG | LEU | 33 | −6.028 | 11.581 | 3.941 | 1.00 | 0.75 | 1SG | 274 |
| ATOM | 274 | CD2 | LEU | 33 | −6.241 | 12.896 | 4.707 | 1.00 | 0.75 | 1SG | 275 |
| ATOM | 275 | CD1 | LEU | 33 | −7.083 | 11.341 | 2.859 | 1.00 | 0.75 | 1SG | 276 |
| ATOM | 276 | C | LEU | 33 | −4.125 | 9.368 | 3.616 | 1.00 | 0.75 | 1SG | 277 |
| ATOM | 277 | O | LEU | 33 | −3.133 | 8.867 | 4.141 | 1.00 | 0.75 | 1SG | 278 |
| ATOM | 278 | N | LYS | 34 | −4.028 | 10.163 | 2.499 | 1.00 | 0.98 | 1SG | 279 |
| ATOM | 279 | CA | LYS | 34 | −2.737 | 10.455 | 1.887 | 1.00 | 0.98 | 1SG | 280 |
| ATOM | 280 | CB | LYS | 34 | −2.043 | 9.154 | 1.409 | 1.00 | 0.98 | 1SG | 281 |
| ATOM | 281 | CG | LYS | 34 | −0.863 | 9.278 | 0.442 | 1.00 | 0.98 | 1SG | 282 |
| ATOM | 282 | CD | LYS | 34 | −1.251 | 9.534 | −1.019 | 1.00 | 0.98 | 1SG | 283 |
| ATOM | 283 | CE | LYS | 34 | −0.051 | 9.652 | −1.960 | 1.00 | 0.98 | 1SG | 284 |
| ATOM | 284 | NZ | LYS | 34 | −0.512 | 9.899 | −3.343 | 1.00 | 0.98 | 1SG | 285 |
| ATOM | 285 | C | LYS | 34 | −2.850 | 11.426 | 0.722 | 1.00 | 0.98 | 1SG | 286 |
| ATOM | 286 | O | LYS | 34 | −3.945 | 11.707 | 0.239 | 1.00 | 0.98 | 1SG | 287 |
| ATOM | 287 | N | CYS | 35 | −1.697 | 11.997 | 0.262 | 1.00 | 1.08 | 1SG | 288 |
| ATOM | 288 | CA | CYS | 35 | −1.654 | 12.849 | −0.911 | 1.00 | 1.08 | 1SG | 289 |
| ATOM | 289 | CB | CYS | 35 | −2.428 | 14.178 | −0.834 | 1.00 | 1.08 | 1SG | 290 |
| ATOM | 290 | SG | CYS | 35 | −1.655 | 15.418 | −1.935 | 1.00 | 1.08 | 1SG | 291 |
| ATOM | 291 | C | CYS | 35 | −0.263 | 13.239 | −1.329 | 1.00 | 1.08 | 1SG | 292 |
| ATOM | 292 | O | CYS | 35 | 0.214 | 12.824 | −2.384 | 1.00 | 1.08 | 1SG | 293 |
| ATOM | 293 | N | ASP | 36 | 0.440 | 14.017 | −0.489 | 1.00 | 0.85 | 1SG | 294 |
| ATOM | 294 | CA | ASP | 36 | 1.638 | 14.705 | −0.888 | 1.00 | 0.85 | 1SG | 295 |
| ATOM | 295 | CB | ASP | 36 | 2.339 | 15.353 | 0.320 | 1.00 | 0.85 | 1SG | 296 |
| ATOM | 296 | CG | ASP | 36 | 3.527 | 16.178 | −0.154 | 1.00 | 0.85 | 1SG | 297 |
| ATOM | 297 | OD1 | ASP | 36 | 3.687 | 16.338 | −1.393 | 1.00 | 0.85 | 1SG | 298 |
| ATOM | 298 | OD2 | ASP | 36 | 4.292 | 16.658 | 0.724 | 1.00 | 0.85 | 1SG | 299 |
| ATOM | 299 | C | ASP | 36 | 2.636 | 13.809 | −1.546 | 1.00 | 0.85 | 1SG | 300 |
| ATOM | 300 | O | ASP | 36 | 3.121 | 14.117 | −2.635 | 1.00 | 0.85 | 1SG | 301 |
| ATOM | 301 | N | SER | 37 | 2.968 | 12.664 | −0.933 | 1.00 | 0.55 | 1SG | 302 |
| ATOM | 302 | CA | SER | 37 | 4.026 | 11.867 | −1.473 | 1.00 | 0.55 | 1SG | 303 |
| ATOM | 303 | CB | SER | 37 | 4.981 | 11.325 | −0.393 | 1.00 | 0.55 | 1SG | 304 |
| ATOM | 304 | OG | SER | 37 | 6.012 | 10.547 | −0.982 | 1.00 | 0.55 | 1SG | 305 |
| ATOM | 305 | C | SER | 37 | 3.454 | 10.698 | −2.194 | 1.00 | 0.55 | 1SG | 306 |
| ATOM | 306 | O | SER | 37 | 2.663 | 9.935 | −1.641 | 1.00 | 0.55 | 1SG | 307 |
| ATOM | 307 | N | ASP | 38 | 3.850 | 10.545 | −3.469 | 1.00 | 0.50 | 1SG | 308 |
| ATOM | 308 | CA | ASP | 38 | 3.409 | 9.427 | −4.245 | 1.00 | 0.50 | 1SG | 309 |
| ATOM | 309 | CB | ASP | 38 | 3.875 | 9.487 | −5.711 | 1.00 | 0.50 | 1SG | 310 |
| ATOM | 310 | CG | ASP | 38 | 3.149 | 8.402 | −6.491 | 1.00 | 0.50 | 1SG | 311 |
| ATOM | 311 | OD1 | ASP | 38 | 2.462 | 7.566 | −5.847 | 1.00 | 0.50 | 1SG | 312 |
| ATOM | 312 | OD2 | ASP | 38 | 3.274 | 8.398 | −7.745 | 1.00 | 0.50 | 1SG | 313 |
| ATOM | 313 | C | ASP | 38 | 4.038 | 8.237 | −3.613 | 1.00 | 0.50 | 1SG | 314 |
| ATOM | 314 | O | ASP | 38 | 3.439 | 7.168 | −3.521 | 1.00 | 0.50 | 1SG | 315 |
| ATOM | 315 | N | HIS | 39 | 5.270 | 8.445 | −3.117 | 1.00 | 0.68 | 1SG | 316 |
| ATOM | 316 | CA | HIS | 39 | 6.095 | 7.451 | −2.500 | 1.00 | 0.68 | 1SG | 317 |
| ATOM | 317 | ND1 | HIS | 39 | 9.227 | 6.426 | −2.704 | 1.00 | 0.68 | 1SG | 318 |
| ATOM | 318 | CG | HIS | 39 | 8.544 | 7.129 | −1.736 | 1.00 | 0.68 | 1SG | 319 |
| ATOM | 319 | CB | HIS | 39 | 7.421 | 8.081 | −2.028 | 1.00 | 0.68 | 1SG | 320 |
| ATOM | 320 | NE2 | HIS | 39 | 10.192 | 5.932 | −0.763 | 1.00 | 0.68 | 1SG | 321 |
| ATOM | 321 | CD2 | HIS | 39 | 9.144 | 6.812 | −0.556 | 1.00 | 0.68 | 1SG | 322 |
| ATOM | 322 | CE1 | HIS | 39 | 10.202 | 5.729 | −2.068 | 1.00 | 0.68 | 1SG | 323 |
| ATOM | 323 | C | HIS | 39 | 5.346 | 6.937 | −1.316 | 1.00 | 0.68 | 1SG | 324 |
| ATOM | 324 | O | HIS | 39 | 5.386 | 5.750 | −0.994 | 1.00 | 0.68 | 1SG | 325 |
| ATOM | 325 | N | LEU | 40 | 4.627 | 7.838 | −0.625 | 1.00 | 0.69 | 1SG | 326 |
| ATOM | 326 | CA | LEU | 40 | 3.878 | 7.436 | 0.523 | 1.00 | 0.69 | 1SG | 327 |
| ATOM | 327 | CB | LEU | 40 | 3.640 | 8.617 | 1.477 | 1.00 | 0.69 | 1SG | 328 |
| ATOM | 328 | CG | LEU | 40 | 4.962 | 9.244 | 1.954 | 1.00 | 0.69 | 1SG | 329 |
| ATOM | 329 | CD2 | LEU | 40 | 5.905 | 8.176 | 2.529 | 1.00 | 0.69 | 1SG | 330 |
| ATOM | 330 | CD1 | LEU | 40 | 4.725 | 10.422 | 2.908 | 1.00 | 0.69 | 1SG | 331 |
| ATOM | 331 | C | LEU | 40 | 2.562 | 6.932 | 0.030 | 1.00 | 0.69 | 1SG | 332 |
| ATOM | 332 | O | LEU | 40 | 2.414 | 6.601 | −1.143 | 1.00 | 0.69 | 1SG | 333 |
| ATOM | 333 | N | GLY | 41 | 1.563 | 6.839 | 0.925 | 1.00 | 0.58 | 1SG | 334 |
| ATOM | 334 | CA | GLY | 41 | 0.279 | 6.384 | 0.480 | 1.00 | 0.58 | 1SG | 335 |
| ATOM | 335 | C | GLY | 41 | 0.058 | 5.015 | 1.027 | 1.00 | 0.58 | 1SG | 336 |
| ATOM | 336 | O | GLY | 41 | 0.957 | 4.403 | 1.603 | 1.00 | 0.58 | 1SG | 337 |
| ATOM | 337 | N | LEU | 42 | −1.171 | 4.503 | 0.847 | 1.00 | 0.76 | 1SG | 338 |
| ATOM | 338 | CA | LEU | 42 | −1.520 | 3.213 | 1.357 | 1.00 | 0.76 | 1SG | 339 |
| ATOM | 339 | CB | LEU | 42 | −2.978 | 2.830 | 1.055 | 1.00 | 0.76 | 1SG | 340 |
| ATOM | 340 | CG | LEU | 42 | −4.005 | 3.751 | 1.742 | 1.00 | 0.76 | 1SG | 341 |
| ATOM | 341 | CD2 | LEU | 42 | −3.880 | 5.203 | 1.252 | 1.00 | 0.76 | 1SG | 342 |
| ATOM | 342 | CD1 | LEU | 42 | −3.926 | 3.626 | 3.272 | 1.00 | 0.76 | 1SG | 343 |
| ATOM | 343 | C | LEU | 42 | −0.640 | 2.202 | 0.707 | 1.00 | 0.76 | 1SG | 344 |
| ATOM | 344 | O | LEU | 42 | 0.021 | 1.419 | 1.382 | 1.00 | 0.76 | 1SG | 345 |
| ATOM | 345 | N | GLU | 43 | −0.586 | 2.194 | −0.636 | 1.00 | 1.04 | 1SG | 346 |
| ATOM | 346 | CA | GLU | 43 | 0.252 | 1.221 | −1.273 | 1.00 | 1.04 | 1SG | 347 |
| ATOM | 347 | CB | GLU | 43 | −0.499 | 0.320 | −2.270 | 1.00 | 1.04 | 1SG | 348 |
| ATOM | 348 | CG | GLU | 43 | 0.399 | −0.718 | −2.951 | 1.00 | 1.04 | 1SG | 349 |
| ATOM | 349 | CD | GLU | 43 | 0.653 | −1.837 | −1.959 | 1.00 | 1.04 | 1SG | 350 |
| ATOM | 350 | OE1 | GLU | 43 | −0.279 | −2.130 | −1.164 | 1.00 | 1.04 | 1SG | 351 |
| ATOM | 351 | OE2 | GLU | 43 | 1.775 | −2.410 | −1.974 | 1.00 | 1.04 | 1SG | 352 |
| ATOM | 352 | C | GLU | 43 | 1.279 | 1.947 | −2.064 | 1.00 | 1.04 | 1SG | 353 |
| ATOM | 353 | O | GLU | 43 | 1.038 | 2.331 | −3.207 | 1.00 | 1.04 | 1SG | 354 |
| ATOM | 354 | N | SER | 44 | 2.471 | 2.171 | −1.475 | 1.00 | 1.17 | 1SG | 355 |
| ATOM | 355 | CA | SER | 44 | 3.462 | 2.812 | −2.273 | 1.00 | 1.17 | 1SG | 356 |
| ATOM | 356 | CB | SER | 44 | 3.347 | 4.336 | −2.364 | 1.00 | 1.17 | 1SG | 357 |
| ATOM | 357 | OG | SER | 44 | 4.402 | 4.807 | −3.189 | 1.00 | 1.17 | 1SG | 358 |
| ATOM | 358 | C | SER | 44 | 4.815 | 2.542 | −1.708 | 1.00 | 1.17 | 1SG | 359 |
| ATOM | 359 | O | SER | 44 | 5.642 | 1.908 | −2.359 | 1.00 | 1.17 | 1SG | 360 |
| ATOM | 360 | N | ARG | 45 | 5.094 | 3.023 | −0.483 | 1.00 | 1.38 | 1SG | 361 |
| ATOM | 361 | CA | ARG | 45 | 6.448 | 2.809 | −0.042 | 1.00 | 1.38 | 1SG | 362 |
| ATOM | 362 | CB | ARG | 45 | 7.411 | 3.823 | −0.666 | 1.00 | 1.38 | 1SG | 363 |
| ATOM | 363 | CG | ARG | 45 | 8.887 | 3.436 | −0.573 | 1.00 | 1.38 | 1SG | 364 |
| ATOM | 364 | CD | ARG | 45 | 9.310 | 2.382 | −1.598 | 1.00 | 1.38 | 1SG | 365 |
| ATOM | 365 | NE | ARG | 45 | 10.800 | 2.350 | −1.610 | 1.00 | 1.38 | 1SG | 366 |
| ATOM | 366 | CZ | ARG | 45 | 11.477 | 1.568 | −0.721 | 1.00 | 1.38 | 1SG | 367 |
| ATOM | 367 | NH1 | ARG | 45 | 10.788 | 0.786 | 0.157 | 1.00 | 1.38 | 1SG | 368 |
| ATOM | 368 | NH2 | ARG | 45 | 12.841 | 1.568 | −0.713 | 1.00 | 1.38 | 1SG | 369 |
| ATOM | 369 | C | ARG | 45 | 6.523 | 2.976 | 1.430 | 1.00 | 1.38 | 1SG | 370 |
| ATOM | 370 | O | ARG | 45 | 5.767 | 2.374 | 2.194 | 1.00 | 1.38 | 1SG | 371 |
| ATOM | 371 | N | GLU | 46 | 7.491 | 3.822 | 1.857 | 1.00 | 1.32 | 1SG | 372 |
| ATOM | 372 | CA | GLU | 46 | 7.785 | 4.130 | 3.217 | 1.00 | 1.32 | 1SG | 373 |
| ATOM | 373 | CB | GLU | 46 | 6.747 | 5.054 | 3.895 | 1.00 | 1.32 | 1SG | 374 |
| ATOM | 374 | CG | GLU | 46 | 5.352 | 4.454 | 4.038 | 1.00 | 1.32 | 1SG | 375 |
| ATOM | 375 | CD | GLU | 46 | 4.545 | 4.758 | 2.785 | 1.00 | 1.32 | 1SG | 376 |
| ATOM | 376 | OE1 | GLU | 46 | 5.129 | 5.319 | 1.821 | 1.00 | 1.32 | 1SG | 377 |
| ATOM | 377 | OE2 | GLU | 46 | 3.331 | 4.420 | 2.771 | 1.00 | 1.32 | 1SG | 378 |
| ATOM | 378 | C | GLU | 46 | 7.875 | 2.888 | 4.027 | 1.00 | 1.32 | 1SG | 379 |
| ATOM | 379 | O | GLU | 46 | 7.747 | 2.916 | 5.254 | 1.00 | 1.32 | 1SG | 380 |
| ATOM | 380 | N | SER | 47 | 8.137 | 1.733 | 3.379 | 1.00 | 0.94 | 1SG | 381 |
| ATOM | 381 | CA | SER | 47 | 8.253 | 0.506 | 4.094 | 1.00 | 0.94 | 1SG | 382 |
| ATOM | 382 | CB | SER | 47 | 7.092 | −0.471 | 3.847 | 1.00 | 0.94 | 1SG | 383 |
| ATOM | 383 | OG | SER | 47 | 7.094 | −0.894 | 2.494 | 1.00 | 0.94 | 1SG | 384 |
| ATOM | 384 | C | SER | 47 | 9.494 | −0.169 | 3.610 | 1.00 | 0.94 | 1SG | 385 |
| ATOM | 385 | O | SER | 47 | 10.152 | 0.318 | 2.695 | 1.00 | 0.94 | 1SG | 386 |
| ATOM | 386 | N | GLN | 48 | 9.856 | −1.294 | 4.243 | 1.00 | 0.80 | 1SG | 387 |
| ATOM | 387 | CA | GLN | 48 | 11.008 | −2.015 | 3.798 | 1.00 | 0.80 | 1SG | 388 |
| ATOM | 388 | CB | GLN | 48 | 11.377 | −3.214 | 4.696 | 1.00 | 0.80 | 1SG | 389 |
| ATOM | 389 | CG | GLN | 48 | 11.919 | −2.833 | 6.076 | 1.00 | 0.80 | 1SG | 390 |
| ATOM | 390 | CD | GLN | 48 | 10.756 | −2.469 | 6.988 | 1.00 | 0.80 | 1SG | 391 |
| ATOM | 391 | OE1 | GLN | 48 | 10.125 | −1.423 | 6.847 | 1.00 | 0.80 | 1SG | 392 |
| ATOM | 392 | NE2 | GLN | 48 | 10.465 | −3.363 | 7.971 | 1.00 | 0.80 | 1SG | 393 |
| ATOM | 393 | C | GLN | 48 | 10.679 | −2.563 | 2.449 | 1.00 | 0.80 | 1SG | 394 |
| ATOM | 394 | O | GLN | 48 | 11.500 | −2.537 | 1.533 | 1.00 | 0.80 | 1SG | 395 |
| ATOM | 395 | N | SER | 49 | 9.436 | −3.054 | 2.291 | 1.00 | 0.77 | 1SG | 396 |
| ATOM | 396 | CA | SER | 49 | 9.028 | −3.633 | 1.047 | 1.00 | 0.77 | 1SG | 397 |
| ATOM | 397 | CB | SER | 49 | 7.599 | −4.206 | 1.070 | 1.00 | 0.77 | 1SG | 398 |
| ATOM | 398 | OG | SER | 49 | 7.526 | −5.289 | 1.986 | 1.00 | 0.77 | 1SG | 399 |
| ATOM | 399 | C | SER | 49 | 9.086 | −2.576 | −0.001 | 1.00 | 0.77 | 1SG | 400 |
| ATOM | 400 | O | SER | 49 | 9.267 | −1.396 | 0.295 | 1.00 | 0.77 | 1SG | 401 |
| ATOM | 401 | N | GLN | 50 | 8.951 | −2.999 | −1.273 | 1.00 | 0.67 | 1SG | 402 |
| ATOM | 402 | CA | GLN | 50 | 9.010 | −2.089 | −2.378 | 1.00 | 0.67 | 1SG | 403 |
| ATOM | 403 | CB | GLN | 50 | 8.938 | −2.815 | −3.735 | 1.00 | 0.67 | 1SG | 404 |
| ATOM | 404 | CG | GLN | 50 | 7.635 | −3.586 | −3.948 | 1.00 | 0.67 | 1SG | 405 |
| ATOM | 405 | CD | GLN | 50 | 7.692 | −4.251 | −5.317 | 1.00 | 0.67 | 1SG | 406 |
| ATOM | 406 | OE1 | GLN | 50 | 8.635 | −4.049 | −6.081 | 1.00 | 0.67 | 1SG | 407 |
| ATOM | 407 | NE2 | GLN | 50 | 6.649 | −5.060 | −5.644 | 1.00 | 0.67 | 1SG | 408 |
| ATOM | 408 | C | GLN | 50 | 7.867 | −1.139 | −2.290 | 1.00 | 0.67 | 1SG | 409 |
| ATOM | 409 | O | GLN | 50 | 8.047 | 0.074 | −2.397 | 1.00 | 0.67 | 1SG | 410 |
| ATOM | 410 | N | TYR | 51 | 6.645 | −1.662 | −2.066 | 1.00 | 0.56 | 1SG | 411 |
| ATOM | 411 | CA | TYR | 51 | 5.499 | −0.795 | −2.001 | 1.00 | 0.56 | 1SG | 412 |
| ATOM | 412 | CB | TYR | 51 | 4.274 | −1.334 | −2.751 | 1.00 | 0.56 | 1SG | 413 |
| ATOM | 413 | CG | TYR | 51 | 4.603 | −1.334 | −4.203 | 1.00 | 0.56 | 1SG | 414 |
| ATOM | 414 | CD1 | TYR | 51 | 4.471 | −0.184 | −4.950 | 1.00 | 0.56 | 1SG | 415 |
| ATOM | 415 | CD2 | TYR | 51 | 5.045 | −2.480 | −4.825 | 1.00 | 0.56 | 1SG | 416 |
| ATOM | 416 | CE1 | TYR | 51 | 4.773 | −0.177 | −6.291 | 1.00 | 0.56 | 1SG | 417 |
| ATOM | 417 | CE2 | TYR | 51 | 5.353 | −2.480 | −6.165 | 1.00 | 0.56 | 1SG | 418 |
| ATOM | 418 | CZ | TYR | 51 | 5.222 | −1.327 | −6.898 | 1.00 | 0.56 | 1SG | 419 |
| ATOM | 419 | OH | TYR | 51 | 5.529 | −1.323 | −8.276 | 1.00 | 0.56 | 1SG | 420 |
| ATOM | 420 | C | TYR | 51 | 5.132 | −0.641 | −0.565 | 1.00 | 0.56 | 1SG | 421 |
| ATOM | 421 | O | TYR | 51 | 5.762 | −1.233 | 0.310 | 1.00 | 0.56 | 1SG | 422 |
| ATOM | 422 | N | CYS | 52 | 4.114 | 0.203 | −0.279 | 1.00 | 0.53 | 1SG | 423 |
| ATOM | 423 | CA | CYS | 52 | 3.690 | 0.356 | 1.070 | 1.00 | 0.53 | 1SG | 424 |
| ATOM | 424 | CB | CYS | 52 | 2.794 | 1.583 | 1.315 | 1.00 | 0.53 | 1SG | 425 |
| ATOM | 425 | SG | CYS | 52 | 2.315 | 1.747 | 3.061 | 1.00 | 0.53 | 1SG | 426 |
| ATOM | 426 | C | CYS | 52 | 2.882 | −0.867 | 1.338 | 1.00 | 0.53 | 1SG | 427 |
| ATOM | 427 | O | CYS | 52 | 1.652 | −0.851 | 1.292 | 1.00 | 0.53 | 1SG | 428 |
| ATOM | 428 | N | ARG | 53 | 3.607 | −1.967 | 1.589 | 1.00 | 0.47 | 1SG | 429 |
| ATOM | 429 | CA | ARG | 53 | 3.099 | −3.266 | 1.895 | 1.00 | 0.47 | 1SG | 430 |
| ATOM | 430 | CB | ARG | 53 | 4.187 | −4.355 | 1.857 | 1.00 | 0.47 | 1SG | 431 |
| ATOM | 431 | CG | ARG | 53 | 3.658 | −5.764 | 2.129 | 1.00 | 0.47 | 1SG | 432 |
| ATOM | 432 | CD | ARG | 53 | 4.743 | −6.840 | 2.102 | 1.00 | 0.47 | 1SG | 433 |
| ATOM | 433 | NE | ARG | 53 | 4.083 | −8.146 | 2.378 | 1.00 | 0.47 | 1SG | 434 |
| ATOM | 434 | CZ | ARG | 53 | 4.841 | −9.254 | 2.621 | 1.00 | 0.47 | 1SG | 435 |
| ATOM | 435 | NH1 | ARG | 53 | 6.202 | −9.161 | 2.627 | 1.00 | 0.47 | 1SG | 436 |
| ATOM | 436 | NH2 | ARG | 53 | 4.236 | −10.456 | 2.851 | 1.00 | 0.47 | 1SG | 437 |
| ATOM | 437 | C | ARG | 53 | 2.505 | −3.270 | 3.261 | 1.00 | 0.47 | 1SG | 438 |
| ATOM | 438 | O | ARG | 53 | 1.587 | −4.037 | 3.542 | 1.00 | 0.47 | 1SG | 439 |
| ATOM | 439 | N | ASN | 54 | 3.024 | −2.410 | 4.147 | 1.00 | 0.47 | 1SG | 440 |
| ATOM | 440 | CA | ASN | 54 | 2.581 | −2.431 | 5.512 | 1.00 | 0.47 | 1SG | 441 |
| ATOM | 441 | CB | ASN | 54 | 3.236 | −1.369 | 6.394 | 1.00 | 0.47 | 1SG | 442 |
| ATOM | 442 | CG | ASN | 54 | 4.689 | −1.774 | 6.582 | 1.00 | 0.47 | 1SG | 443 |
| ATOM | 443 | OD1 | ASN | 54 | 5.441 | −1.934 | 5.621 | 1.00 | 0.47 | 1SG | 444 |
| ATOM | 444 | ND2 | ASN | 54 | 5.094 | −1.950 | 7.868 | 1.00 | 0.47 | 1SG | 445 |
| ATOM | 445 | C | ASN | 54 | 1.106 | −2.214 | 5.546 | 1.00 | 0.47 | 1SG | 446 |
| ATOM | 446 | O | ASN | 54 | 0.413 | −2.798 | 6.379 | 1.00 | 0.47 | 1SG | 447 |
| ATOM | 447 | N | ILE | 55 | 0.584 | −1.377 | 4.633 | 1.00 | 0.61 | 1SG | 448 |
| ATOM | 448 | CA | ILE | 55 | −0.823 | −1.122 | 4.628 | 1.00 | 0.61 | 1SG | 449 |
| ATOM | 449 | CB | ILE | 55 | −1.242 | −0.214 | 3.501 | 1.00 | 0.61 | 1SG | 450 |
| ATOM | 450 | CG2 | ILE | 55 | −1.061 | −0.973 | 2.174 | 1.00 | 0.61 | 1SG | 451 |
| ATOM | 451 | CG1 | ILE | 55 | −2.669 | 0.333 | 3.703 | 1.00 | 0.61 | 1SG | 452 |
| ATOM | 452 | CD1 | ILE | 55 | −3.768 | −0.726 | 3.627 | 1.00 | 0.61 | 1SG | 453 |
| ATOM | 453 | C | ILE | 55 | −1.520 | −2.441 | 4.474 | 1.00 | 0.61 | 1SG | 454 |
| ATOM | 454 | O | ILE | 55 | −2.547 | −2.683 | 5.103 | 1.00 | 0.61 | 1SG | 455 |
| ATOM | 455 | N | LEU | 56 | −0.932 | −3.354 | 3.681 | 1.00 | 0.51 | 1SG | 456 |
| ATOM | 456 | CA | LEU | 56 | −1.510 | −4.634 | 3.396 | 1.00 | 0.51 | 1SG | 457 |
| ATOM | 457 | CB | LEU | 56 | −0.649 | −5.499 | 2.459 | 1.00 | 0.51 | 1SG | 458 |
| ATOM | 458 | CG | LEU | 56 | −0.488 | −4.907 | 1.049 | 1.00 | 0.51 | 1SG | 459 |
| ATOM | 459 | CD2 | LEU | 56 | 0.138 | −5.926 | 0.083 | 1.00 | 0.51 | 1SG | 460 |
| ATOM | 460 | CD1 | LEU | 56 | 0.279 | −3.576 | 1.100 | 1.00 | 0.51 | 1SG | 461 |
| ATOM | 461 | C | LEU | 56 | −1.663 | −5.401 | 4.669 | 1.00 | 0.51 | 1SG | 462 |
| ATOM | 462 | O | LEU | 56 | −2.607 | −6.176 | 4.813 | 1.00 | 0.51 | 1SG | 463 |
| ATOM | 463 | N | TYR | 57 | −0.742 | −5.198 | 5.630 | 1.00 | 0.40 | 1SG | 464 |
| ATOM | 464 | CA | TYR | 57 | −0.773 | −5.925 | 6.869 | 1.00 | 0.40 | 1SG | 465 |
| ATOM | 465 | CB | TYR | 57 | 0.257 | −5.433 | 7.902 | 1.00 | 0.40 | 1SG | 466 |
| ATOM | 466 | CG | TYR | 57 | 1.604 | −5.889 | 7.457 | 1.00 | 0.40 | 1SG | 467 |
| ATOM | 467 | CD1 | TYR | 57 | 2.303 | −5.199 | 6.494 | 1.00 | 0.40 | 1SG | 468 |
| ATOM | 468 | CD2 | TYR | 57 | 2.169 | −7.013 | 8.016 | 1.00 | 0.40 | 1SG | 469 |
| ATOM | 469 | CE1 | TYR | 57 | 3.547 | −5.627 | 6.091 | 1.00 | 0.40 | 1SG | 470 |
| ATOM | 470 | CE2 | TYR | 57 | 3.411 | −7.444 | 7.617 | 1.00 | 0.40 | 1SG | 471 |
| ATOM | 471 | CZ | TYR | 57 | 4.102 | −6.751 | 6.653 | 1.00 | 0.40 | 1SG | 472 |
| ATOM | 472 | OH | TYR | 57 | 5.376 | −7.194 | 6.242 | 1.00 | 0.40 | 1SG | 473 |
| ATOM | 473 | C | TYR | 57 | −2.126 | −5.776 | 7.481 | 1.00 | 0.40 | 1SG | 474 |
| ATOM | 474 | O | TYR | 57 | −2.590 | −6.666 | 8.192 | 1.00 | 0.40 | 1SG | 475 |
| ATOM | 475 | N | ASN | 58 | −2.796 | −4.642 | 7.215 | 1.00 | 0.69 | 1SG | 476 |
| ATOM | 476 | CA | ASN | 58 | −4.098 | −4.423 | 7.773 | 1.00 | 0.69 | 1SG | 477 |
| ATOM | 477 | CB | ASN | 58 | −4.792 | −3.152 | 7.246 | 1.00 | 0.69 | 1SG | 478 |
| ATOM | 478 | CG | ASN | 58 | −5.155 | −3.346 | 5.772 | 1.00 | 0.69 | 1SG | 479 |
| ATOM | 479 | OD1 | ASN | 58 | −4.596 | −4.172 | 5.052 | 1.00 | 0.69 | 1SG | 480 |
| ATOM | 480 | ND2 | ASN | 58 | −6.145 | −2.542 | 5.299 | 1.00 | 0.69 | 1SG | 481 |
| ATOM | 481 | C | ASN | 58 | −4.968 | −5.569 | 7.376 | 1.00 | 0.69 | 1SG | 482 |
| ATOM | 482 | O | ASN | 58 | −4.652 | −6.309 | 6.445 | 1.00 | 0.69 | 1SG | 483 |
| ATOM | 483 | N | PHE | 59 | −6.083 | −5.765 | 8.104 | 1.00 | 0.93 | 1SG | 484 |
| ATOM | 484 | CA | PHE | 59 | −6.993 | −6.798 | 7.717 | 1.00 | 0.93 | 1SG | 485 |
| ATOM | 485 | CB | PHE | 59 | −7.300 | −7.835 | 8.821 | 1.00 | 0.93 | 1SG | 486 |
| ATOM | 486 | CG | PHE | 59 | −7.813 | −7.183 | 10.061 | 1.00 | 0.93 | 1SG | 487 |
| ATOM | 487 | CD1 | PHE | 59 | −6.937 | −6.596 | 10.945 | 1.00 | 0.93 | 1SG | 488 |
| ATOM | 488 | CD2 | PHE | 59 | −9.157 | −7.176 | 10.356 | 1.00 | 0.93 | 1SG | 489 |
| ATOM | 489 | CE1 | PHE | 59 | −7.391 | −6.003 | 12.100 | 1.00 | 0.93 | 1SG | 490 |
| ATOM | 490 | CE2 | PHE | 59 | −9.621 | −6.586 | 11.507 | 1.00 | 0.93 | 1SG | 491 |
| ATOM | 491 | CZ | PHE | 59 | −8.737 | −6.002 | 12.382 | 1.00 | 0.93 | 1SG | 492 |
| ATOM | 492 | C | PHE | 59 | −8.251 | −6.146 | 7.235 | 1.00 | 0.93 | 1SG | 493 |
| ATOM | 493 | O | PHE | 59 | −8.207 | −5.131 | 6.543 | 1.00 | 0.93 | 1SG | 494 |
| ATOM | 494 | N | LEU | 60 | −9.413 | −6.730 | 7.580 | 1.00 | 0.81 | 1SG | 495 |
| ATOM | 495 | CA | LEU | 60 | −10.688 | −6.241 | 7.141 | 1.00 | 0.81 | 1SG | 496 |
| ATOM | 496 | CB | LEU | 60 | −11.853 | −7.121 | 7.625 | 1.00 | 0.81 | 1SG | 497 |
| ATOM | 497 | CG | LEU | 60 | −11.761 | −8.594 | 7.188 | 1.00 | 0.81 | 1SG | 498 |
| ATOM | 498 | CD2 | LEU | 60 | −13.092 | −9.322 | 7.429 | 1.00 | 0.81 | 1SG | 499 |
| ATOM | 499 | CD1 | LEU | 60 | −10.564 | −9.303 | 7.845 | 1.00 | 0.81 | 1SG | 500 |
| ATOM | 500 | C | LEU | 60 | −10.922 | −4.889 | 7.730 | 1.00 | 0.81 | 1SG | 501 |
| ATOM | 501 | O | LEU | 60 | −11.477 | −3.996 | 7.092 | 1.00 | 0.81 | 1SG | 502 |
| ATOM | 502 | N | LYS | 61 | −10.461 | −4.696 | 8.973 | 1.00 | 0.65 | 1SG | 503 |
| ATOM | 503 | CA | LYS | 61 | −10.774 | −3.505 | 9.696 | 1.00 | 0.65 | 1SG | 504 |
| ATOM | 504 | CB | LYS | 61 | −10.150 | −3.477 | 11.103 | 1.00 | 0.65 | 1SG | 505 |
| ATOM | 505 | CG | LYS | 61 | −8.630 | −3.340 | 11.120 | 1.00 | 0.65 | 1SG | 506 |
| ATOM | 506 | CD | LYS | 61 | −8.051 | −3.121 | 12.520 | 1.00 | 0.65 | 1SG | 507 |
| ATOM | 507 | CE | LYS | 61 | −8.870 | −2.158 | 13.381 | 1.00 | 0.65 | 1SG | 508 |
| ATOM | 508 | NZ | LYS | 61 | −10.025 | −2.873 | 13.972 | 1.00 | 0.65 | 1SG | 509 |
| ATOM | 509 | C | LYS | 61 | −10.350 | −2.285 | 8.941 | 1.00 | 0.65 | 1SG | 510 |
| ATOM | 510 | O | LYS | 61 | −11.006 | −1.253 | 9.060 | 1.00 | 0.65 | 1SG | 511 |
| ATOM | 511 | N | LEU | 62 | −9.258 | −2.338 | 8.149 | 1.00 | 0.64 | 1SG | 512 |
| ATOM | 512 | CA | LEU | 62 | −8.865 | −1.123 | 7.486 | 1.00 | 0.64 | 1SG | 513 |
| ATOM | 513 | CB | LEU | 62 | −7.344 | −0.889 | 7.407 | 1.00 | 0.64 | 1SG | 514 |
| ATOM | 514 | CG | LEU | 62 | −6.609 | −0.794 | 8.754 | 1.00 | 0.64 | 1SG | 515 |
| ATOM | 515 | CD2 | LEU | 62 | −5.236 | −0.125 | 8.585 | 1.00 | 0.64 | 1SG | 516 |
| ATOM | 516 | CD1 | LEU | 62 | −6.519 | −2.161 | 9.444 | 1.00 | 0.64 | 1SG | 517 |
| ATOM | 517 | C | LEU | 62 | −9.300 | −1.142 | 6.054 | 1.00 | 0.64 | 1SG | 518 |
| ATOM | 518 | O | LEU | 62 | −8.821 | −1.950 | 5.260 | 1.00 | 0.64 | 1SG | 519 |
| ATOM | 519 | N | PRO | 63 | −10.211 | −0.276 | 5.700 | 1.00 | 0.60 | 1SG | 520 |
| ATOM | 520 | CA | PRO | 63 | −10.519 | −0.144 | 4.305 | 1.00 | 0.60 | 1SG | 521 |
| ATOM | 521 | CD | PRO | 63 | −11.392 | −0.085 | 6.527 | 1.00 | 0.60 | 1SG | 522 |
| ATOM | 522 | CB | PRO | 63 | −11.931 | 0.420 | 4.228 | 1.00 | 0.60 | 1SG | 523 |
| ATOM | 523 | CG | PRO | 63 | −12.576 | −0.003 | 5.555 | 1.00 | 0.60 | 1SG | 524 |
| ATOM | 524 | C | PRO | 63 | −9.488 | 0.801 | 3.788 | 1.00 | 0.60 | 1SG | 525 |
| ATOM | 525 | O | PRO | 63 | −8.878 | 1.497 | 4.598 | 1.00 | 0.60 | 1SG | 526 |
| ATOM | 526 | N | ALA | 64 | −9.246 | 0.849 | 2.467 | 1.00 | 0.40 | 1SG | 527 |
| ATOM | 527 | CA | ALA | 64 | −8.254 | 1.781 | 2.028 | 1.00 | 0.40 | 1SG | 528 |
| ATOM | 528 | CB | ALA | 64 | −7.192 | 1.156 | 1.109 | 1.00 | 0.40 | 1SG | 529 |
| ATOM | 529 | C | ALA | 64 | −8.950 | 2.842 | 1.242 | 1.00 | 0.40 | 1SG | 530 |
| ATOM | 530 | O | ALA | 64 | −9.348 | 2.623 | 0.099 | 1.00 | 0.40 | 1SG | 531 |
| ATOM | 531 | N | LYS | 65 | −9.126 | 4.027 | 1.853 | 1.00 | 0.51 | 1SG | 532 |
| ATOM | 532 | CA | LYS | 65 | −9.733 | 5.121 | 1.156 | 1.00 | 0.51 | 1SG | 533 |
| ATOM | 533 | CB | LYS | 65 | −10.958 | 5.729 | 1.870 | 1.00 | 0.51 | 1SG | 534 |
| ATOM | 534 | CG | LYS | 65 | −12.292 | 5.032 | 1.586 | 1.00 | 0.51 | 1SG | 535 |
| ATOM | 535 | CD | LYS | 65 | −12.436 | 3.612 | 2.136 | 1.00 | 0.51 | 1SG | 536 |
| ATOM | 536 | CE | LYS | 65 | −13.799 | 2.997 | 1.805 | 1.00 | 0.51 | 1SG | 537 |
| ATOM | 537 | NZ | LYS | 65 | −13.895 | 1.623 | 2.338 | 1.00 | 0.51 | 1SG | 538 |
| ATOM | 538 | C | LYS | 65 | −8.703 | 6.192 | 1.083 | 1.00 | 0.51 | 1SG | 539 |
| ATOM | 539 | O | LYS | 65 | −8.231 | 6.683 | 2.104 | 1.00 | 0.51 | 1SG | 540 |
| ATOM | 540 | N | ARG | 66 | −8.315 | 6.589 | −0.137 | 1.00 | 0.72 | 1SG | 541 |
| ATOM | 541 | CA | ARG | 66 | −7.321 | 7.611 | −0.220 | 1.00 | 0.72 | 1SG | 542 |
| ATOM | 542 | CB | ARG | 66 | −6.212 | 7.295 | −1.242 | 1.00 | 0.72 | 1SG | 543 |
| ATOM | 543 | CG | ARG | 66 | −5.136 | 8.378 | −1.350 | 1.00 | 0.72 | 1SG | 544 |
| ATOM | 544 | CD | ARG | 66 | −4.009 | 8.017 | −2.319 | 1.00 | 0.72 | 1SG | 545 |
| ATOM | 545 | NE | ARG | 66 | −3.263 | 6.871 | −1.725 | 1.00 | 0.72 | 1SG | 546 |
| ATOM | 546 | CZ | ARG | 66 | −2.820 | 5.855 | −2.521 | 1.00 | 0.72 | 1SG | 547 |
| ATOM | 547 | NH1 | ARG | 66 | −3.078 | 5.877 | −3.862 | 1.00 | 0.72 | 1SG | 548 |
| ATOM | 548 | NH2 | ARG | 66 | −2.120 | 4.818 | −1.975 | 1.00 | 0.72 | 1SG | 549 |
| ATOM | 549 | C | ARG | 66 | −7.992 | 8.872 | −0.657 | 1.00 | 0.72 | 1SG | 550 |
| ATOM | 550 | O | ARG | 66 | −8.517 | 8.949 | −1.766 | 1.00 | 0.72 | 1SG | 551 |
| ATOM | 551 | N | SER | 67 | −8.014 | 9.893 | 0.227 | 1.00 | 0.64 | 1SG | 552 |
| ATOM | 552 | CA | SER | 67 | −8.534 | 11.173 | −0.156 | 1.00 | 0.64 | 1SG | 553 |
| ATOM | 553 | CB | SER | 67 | −9.253 | 11.945 | 0.961 | 1.00 | 0.64 | 1SG | 554 |
| ATOM | 554 | OG | SER | 67 | −10.453 | 11.282 | 1.324 | 1.00 | 0.64 | 1SG | 555 |
| ATOM | 555 | C | SER | 67 | −7.323 | 11.949 | −0.509 | 1.00 | 0.64 | 1SG | 556 |
| ATOM | 556 | O | SER | 67 | −6.441 | 12.154 | 0.322 | 1.00 | 0.64 | 1SG | 557 |
| ATOM | 557 | N | ILE | 68 | −7.241 | 12.404 | −1.765 | 1.00 | 0.58 | 1SG | 558 |
| ATOM | 558 | CA | ILE | 68 | −6.010 | 13.016 | −2.143 | 1.00 | 0.58 | 1SG | 559 |
| ATOM | 559 | CB | ILE | 68 | −5.370 | 12.311 | −3.302 | 1.00 | 0.58 | 1SG | 560 |
| ATOM | 560 | CG2 | ILE | 68 | −6.286 | 12.489 | −4.525 | 1.00 | 0.58 | 1SG | 561 |
| ATOM | 561 | CG1 | ILE | 68 | −3.934 | 12.793 | −3.520 | 1.00 | 0.58 | 1SG | 562 |
| ATOM | 562 | CD1 | ILE | 68 | −3.147 | 11.922 | −4.499 | 1.00 | 0.58 | 1SG | 563 |
| ATOM | 563 | C | ILE | 68 | −6.257 | 14.417 | −2.572 | 1.00 | 0.58 | 1SG | 564 |
| ATOM | 564 | O | ILE | 68 | −7.251 | 14.712 | −3.231 | 1.00 | 0.58 | 1SG | 565 |
| ATOM | 565 | N | ASN | 69 | −5.353 | 15.329 | −2.164 | 1.00 | 0.86 | 1SG | 566 |
| ATOM | 566 | CA | ASN | 69 | −5.415 | 16.668 | −2.661 | 1.00 | 0.86 | 1SG | 567 |
| ATOM | 567 | CB | ASN | 69 | −5.274 | 17.767 | −1.579 | 1.00 | 0.86 | 1SG | 568 |
| ATOM | 568 | CG | ASN | 69 | −3.970 | 17.637 | −0.801 | 1.00 | 0.86 | 1SG | 569 |
| ATOM | 569 | OD1 | ASN | 69 | −3.640 | 16.573 | −0.284 | 1.00 | 0.86 | 1SG | 570 |
| ATOM | 570 | ND2 | ASN | 69 | −3.207 | 18.759 | −0.712 | 1.00 | 0.86 | 1SG | 571 |
| ATOM | 571 | C | ASN | 69 | −4.276 | 16.784 | −3.620 | 1.00 | 0.86 | 1SG | 572 |
| ATOM | 572 | O | ASN | 69 | −3.395 | 17.627 | −3.472 | 1.00 | 0.86 | 1SG | 573 |
| ATOM | 573 | N | CYS | 70 | −4.267 | 15.915 | −4.647 | 1.00 | 1.19 | 1SG | 574 |
| ATOM | 574 | CA | CYS | 70 | −3.202 | 15.945 | −5.601 | 1.00 | 1.19 | 1SG | 575 |
| ATOM | 575 | CB | CYS | 70 | −1.817 | 15.934 | −4.942 | 1.00 | 1.19 | 1SG | 576 |
| ATOM | 576 | SG | CYS | 70 | −1.505 | 14.566 | −3.784 | 1.00 | 1.19 | 1SG | 577 |
| ATOM | 577 | C | CYS | 70 | −3.347 | 14.746 | −6.487 | 1.00 | 1.19 | 1SG | 578 |
| ATOM | 578 | O | CYS | 70 | −4.438 | 14.196 | −6.622 | 1.00 | 1.19 | 1SG | 579 |
| ATOM | 579 | N | SER | 71 | −2.248 | 14.321 | −7.143 | 1.00 | 1.04 | 1SG | 580 |
| ATOM | 580 | CA | SER | 71 | −2.335 | 13.189 | −8.024 | 1.00 | 1.04 | 1SG | 581 |
| ATOM | 581 | CB | SER | 71 | −1.805 | 13.466 | −9.442 | 1.00 | 1.04 | 1SG | 582 |
| ATOM | 582 | OG | SER | 71 | −2.610 | 14.443 | −10.085 | 1.00 | 1.04 | 1SG | 583 |
| ATOM | 583 | C | SER | 71 | −1.515 | 12.070 | −7.455 | 1.00 | 1.04 | 1SG | 584 |
| ATOM | 584 | O | SER | 71 | −0.775 | 12.254 | −6.490 | 1.00 | 1.04 | 1SG | 585 |
| ATOM | 585 | N | GLY | 72 | −1.644 | 10.862 | −8.049 | 1.00 | 0.81 | 1SG | 586 |
| ATOM | 586 | CA | GLY | 72 | −0.919 | 9.705 | −7.599 | 1.00 | 0.81 | 1SG | 587 |
| ATOM | 587 | C | GLY | 72 | −1.941 | 8.681 | −7.228 | 1.00 | 0.81 | 1SG | 588 |
| ATOM | 588 | O | GLY | 72 | −2.429 | 8.652 | −6.100 | 1.00 | 0.81 | 1SG | 589 |
| ATOM | 589 | N | VAL | 73 | −2.279 | 7.791 | −8.179 | 1.00 | 0.91 | 1SG | 590 |
| ATOM | 590 | CA | VAL | 73 | −3.310 | 6.825 | −7.938 | 1.00 | 0.91 | 1SG | 591 |
| ATOM | 591 | CB | VAL | 73 | −3.938 | 6.307 | −9.199 | 1.00 | 0.91 | 1SG | 592 |
| ATOM | 592 | CG1 | VAL | 73 | −4.626 | 7.477 | −9.923 | 1.00 | 0.91 | 1SG | 593 |
| ATOM | 593 | CG2 | VAL | 73 | −2.852 | 5.609 | −10.035 | 1.00 | 0.91 | 1SG | 594 |
| ATOM | 594 | C | VAL | 73 | −2.728 | 5.660 | −7.223 | 1.00 | 0.91 | 1SG | 595 |
| ATOM | 595 | O | VAL | 73 | −1.808 | 5.789 | −6.419 | 1.00 | 0.91 | 1SG | 596 |
| ATOM | 596 | N | THR | 74 | −3.294 | 4.470 | −7.485 | 1.00 | 0.86 | 1SG | 597 |
| ATOM | 597 | CA | THR | 74 | −2.807 | 3.297 | −6.838 | 1.00 | 0.86 | 1SG | 598 |
| ATOM | 598 | CB | THR | 74 | −3.660 | 2.870 | −5.678 | 1.00 | 0.86 | 1SG | 599 |
| ATOM | 599 | OG1 | THR | 74 | −2.998 | 1.877 | −4.908 | 1.00 | 0.86 | 1SG | 600 |
| ATOM | 600 | CG2 | THR | 74 | −4.992 | 2.326 | −6.223 | 1.00 | 0.86 | 1SG | 601 |
| ATOM | 601 | C | THR | 74 | −2.843 | 2.205 | −7.848 | 1.00 | 0.86 | 1SG | 602 |
| ATOM | 602 | O | THR | 74 | −2.998 | 2.452 | −9.043 | 1.00 | 0.86 | 1SG | 603 |
| ATOM | 603 | N | ARG | 75 | −2.687 | 0.954 | −7.383 | 1.00 | 0.52 | 1SG | 604 |
| ATOM | 604 | CA | ARG | 75 | −2.731 | −0.154 | −8.282 | 1.00 | 0.52 | 1SG | 605 |
| ATOM | 605 | CB | ARG | 75 | −2.642 | −1.507 | −7.560 | 1.00 | 0.52 | 1SG | 606 |
| ATOM | 606 | CG | ARG | 75 | −1.305 | −1.757 | −6.863 | 1.00 | 0.52 | 1SG | 607 |
| ATOM | 607 | CD | ARG | 75 | −1.271 | −3.079 | −6.093 | 1.00 | 0.52 | 1SG | 608 |
| ATOM | 608 | NE | ARG | 75 | 0.074 | −3.204 | −5.465 | 1.00 | 0.52 | 1SG | 609 |
| ATOM | 609 | CZ | ARG | 75 | 1.081 | −3.829 | −6.142 | 1.00 | 0.52 | 1SG | 610 |
| ATOM | 610 | NH1 | ARG | 75 | 0.851 | −4.330 | −7.390 | 1.00 | 0.52 | 1SG | 611 |
| ATOM | 611 | NH2 | ARG | 75 | 2.315 | −3.952 | −5.573 | 1.00 | 0.52 | 1SG | 612 |
| ATOM | 612 | C | ARG | 75 | −4.069 | −0.098 | −8.933 | 1.00 | 0.52 | 1SG | 613 |
| ATOM | 613 | O | ARG | 75 | −4.180 | −0.214 | −10.152 | 1.00 | 0.52 | 1SG | 614 |
| ATOM | 614 | N | GLY | 76 | −5.129 | 0.109 | −8.125 | 1.00 | 0.35 | 1SG | 615 |
| ATOM | 615 | CA | GLY | 76 | −6.431 | 0.183 | −8.711 | 1.00 | 0.35 | 1SG | 616 |
| ATOM | 616 | C | GLY | 76 | −7.454 | −0.163 | −7.676 | 1.00 | 0.35 | 1SG | 617 |
| ATOM | 617 | O | GLY | 76 | −7.839 | 0.668 | −6.856 | 1.00 | 0.35 | 1SG | 618 |
| ATOM | 618 | N | ASP | 77 | −7.920 | −1.425 | −7.711 | 1.00 | 0.46 | 1SG | 619 |
| ATOM | 619 | CA | ASP | 77 | −8.965 | −1.920 | −6.862 | 1.00 | 0.46 | 1SG | 620 |
| ATOM | 620 | CB | ASP | 77 | −9.389 | −3.358 | −7.214 | 1.00 | 0.46 | 1SG | 621 |
| ATOM | 621 | CG | ASP | 77 | −8.202 | −4.296 | −7.030 | 1.00 | 0.46 | 1SG | 622 |
| ATOM | 622 | OD1 | ASP | 77 | −7.039 | −3.826 | −7.149 | 1.00 | 0.46 | 1SG | 623 |
| ATOM | 623 | OD2 | ASP | 77 | −8.447 | −5.504 | −6.771 | 1.00 | 0.46 | 1SG | 624 |
| ATOM | 624 | C | ASP | 77 | −8.549 | −1.905 | −5.426 | 1.00 | 0.46 | 1SG | 625 |
| ATOM | 625 | O | ASP | 77 | −9.369 | −1.656 | −4.544 | 1.00 | 0.46 | 1SG | 626 |
| ATOM | 626 | N | GLN | 78 | −7.259 | −2.151 | −5.144 | 1.00 | 0.55 | 1SG | 627 |
| ATOM | 627 | CA | GLN | 78 | −6.827 | −2.270 | −3.782 | 1.00 | 0.55 | 1SG | 628 |
| ATOM | 628 | CB | GLN | 78 | −5.312 | −2.498 | −3.658 | 1.00 | 0.55 | 1SG | 629 |
| ATOM | 629 | CG | GLN | 78 | −4.832 | −2.633 | −2.213 | 1.00 | 0.55 | 1SG | 630 |
| ATOM | 630 | CD | GLN | 78 | −3.327 | −2.854 | −2.239 | 1.00 | 0.55 | 1SG | 631 |
| ATOM | 631 | OE1 | GLN | 78 | −2.690 | −2.771 | −3.287 | 1.00 | 0.55 | 1SG | 632 |
| ATOM | 632 | NE2 | GLN | 78 | −2.738 | −3.148 | −1.049 | 1.00 | 0.55 | 1SG | 633 |
| ATOM | 633 | C | GLN | 78 | −7.160 | −1.013 | −3.046 | 1.00 | 0.55 | 1SG | 634 |
| ATOM | 634 | O | GLN | 78 | −7.632 | −1.064 | −1.910 | 1.00 | 0.55 | 1SG | 635 |
| ATOM | 635 | N | GLU | 79 | −6.937 | 0.154 | −3.676 | 1.00 | 0.49 | 1SG | 636 |
| ATOM | 636 | CA | GLU | 79 | −7.184 | 1.394 | −3.002 | 1.00 | 0.49 | 1SG | 637 |
| ATOM | 637 | CB | GLU | 79 | −6.025 | 2.394 | −3.172 | 1.00 | 0.49 | 1SG | 638 |
| ATOM | 638 | CG | GLU | 79 | −6.243 | 3.734 | −2.470 | 1.00 | 0.49 | 1SG | 639 |
| ATOM | 639 | CD | GLU | 79 | −5.911 | 3.550 | −0.997 | 1.00 | 0.49 | 1SG | 640 |
| ATOM | 640 | OE1 | GLU | 79 | −5.467 | 2.431 | −0.627 | 1.00 | 0.49 | 1SG | 641 |
| ATOM | 641 | OE2 | GLU | 79 | −6.094 | 4.527 | −0.224 | 1.00 | 0.49 | 1SG | 642 |
| ATOM | 642 | C | GLU | 79 | −8.395 | 2.034 | −3.603 | 1.00 | 0.49 | 1SG | 643 |
| ATOM | 643 | O | GLU | 79 | −8.730 | 1.800 | −4.763 | 1.00 | 0.49 | 1SG | 644 |
| ATOM | 644 | N | ALA | 80 | −9.108 | 2.841 | −2.793 | 1.00 | 0.38 | 1SG | 645 |
| ATOM | 645 | CA | ALA | 80 | −10.231 | 3.579 | −3.289 | 1.00 | 0.38 | 1SG | 646 |
| ATOM | 646 | CB | ALA | 80 | −11.467 | 3.525 | −2.372 | 1.00 | 0.38 | 1SG | 647 |
| ATOM | 647 | C | ALA | 80 | −9.764 | 4.994 | −3.327 | 1.00 | 0.38 | 1SG | 648 |
| ATOM | 648 | O | ALA | 80 | −9.334 | 5.535 | −2.310 | 1.00 | 0.38 | 1SG | 649 |
| ATOM | 649 | N | VAL | 81 | −9.818 | 5.639 | −4.507 | 1.00 | 0.41 | 1SG | 650 |
| ATOM | 650 | CA | VAL | 81 | −9.281 | 6.963 | −4.546 | 1.00 | 0.41 | 1SG | 651 |
| ATOM | 651 | CB | VAL | 81 | −8.235 | 7.155 | −5.605 | 1.00 | 0.41 | 1SG | 652 |
| ATOM | 652 | CG1 | VAL | 81 | −8.885 | 6.951 | −6.983 | 1.00 | 0.41 | 1SG | 653 |
| ATOM | 653 | CG2 | VAL | 81 | −7.604 | 8.545 | −5.416 | 1.00 | 0.41 | 1SG | 654 |
| ATOM | 654 | C | VAL | 81 | −10.365 | 7.951 | −4.810 | 1.00 | 0.41 | 1SG | 655 |
| ATOM | 655 | O | VAL | 81 | −11.174 | 7.794 | −5.723 | 1.00 | 0.41 | 1SG | 656 |
| ATOM | 656 | N | LEU | 82 | −10.407 | 9.003 | −3.971 | 1.00 | 0.47 | 1SG | 657 |
| ATOM | 657 | CA | LEU | 82 | −11.329 | 10.078 | −4.164 | 1.00 | 0.47 | 1SG | 658 |
| ATOM | 658 | CB | LEU | 82 | −12.404 | 10.183 | −3.069 | 1.00 | 0.47 | 1SG | 659 |
| ATOM | 659 | CG | LEU | 82 | −13.319 | 8.945 | −3.008 | 1.00 | 0.47 | 1SG | 660 |
| ATOM | 660 | CD2 | LEU | 82 | −14.580 | 9.214 | −2.172 | 1.00 | 0.47 | 1SG | 661 |
| ATOM | 661 | CD1 | LEU | 82 | −12.544 | 7.700 | −2.546 | 1.00 | 0.47 | 1SG | 662 |
| ATOM | 662 | C | LEU | 82 | −10.487 | 11.309 | −4.116 | 1.00 | 0.47 | 1SG | 663 |
| ATOM | 663 | O | LEU | 82 | −9.598 | 11.426 | −3.273 | 1.00 | 0.47 | 1SG | 664 |
| ATOM | 664 | N | GLN | 83 | −10.724 | 12.263 | −5.032 | 1.00 | 0.44 | 1SG | 665 |
| ATOM | 665 | CA | GLN | 83 | −9.872 | 13.413 | −5.024 | 1.00 | 0.44 | 1SG | 666 |
| ATOM | 666 | CB | GLN | 83 | −9.205 | 13.715 | −6.376 | 1.00 | 0.44 | 1SG | 667 |
| ATOM | 667 | CG | GLN | 83 | −8.305 | 14.952 | −6.325 | 1.00 | 0.44 | 1SG | 668 |
| ATOM | 668 | CD | GLN | 83 | −7.806 | 15.251 | −7.731 | 1.00 | 0.44 | 1SG | 669 |
| ATOM | 669 | OE1 | GLN | 83 | −7.449 | 14.350 | −8.487 | 1.00 | 0.44 | 1SG | 670 |
| ATOM | 670 | NE2 | GLN | 83 | −7.788 | 16.562 | −8.097 | 1.00 | 0.44 | 1SG | 671 |
| ATOM | 671 | C | GLN | 83 | −10.676 | 14.616 | −4.692 | 1.00 | 0.44 | 1SG | 672 |
| ATOM | 672 | O | GLN | 83 | −11.814 | 14.769 | −5.133 | 1.00 | 0.44 | 1SG | 673 |
| ATOM | 673 | N | ALA | 84 | −10.088 | 15.505 | −3.873 | 1.00 | 0.59 | 1SG | 674 |
| ATOM | 674 | CA | ALA | 84 | −10.736 | 16.742 | −3.586 | 1.00 | 0.59 | 1SG | 675 |
| ATOM | 675 | CB | ALA | 84 | −10.372 | 17.325 | −2.210 | 1.00 | 0.59 | 1SG | 676 |
| ATOM | 676 | C | ALA | 84 | −10.217 | 17.682 | −4.619 | 1.00 | 0.59 | 1SG | 677 |
| ATOM | 677 | O | ALA | 84 | −9.012 | 17.905 | −4.713 | 1.00 | 0.59 | 1SG | 678 |
| ATOM | 678 | N | ILE | 85 | −11.112 | 18.245 | −5.446 | 1.00 | 0.80 | 1SG | 679 |
| ATOM | 679 | CA | ILE | 85 | −10.616 | 19.127 | −6.456 | 1.00 | 0.80 | 1SG | 680 |
| ATOM | 680 | CB | ILE | 85 | −11.663 | 19.625 | −7.413 | 1.00 | 0.80 | 1SG | 681 |
| ATOM | 681 | CG2 | ILE | 85 | −12.195 | 18.415 | −8.199 | 1.00 | 0.80 | 1SG | 682 |
| ATOM | 682 | CG1 | ILE | 85 | −12.750 | 20.434 | −6.680 | 1.00 | 0.80 | 1SG | 683 |
| ATOM | 683 | CD1 | ILE | 85 | −13.566 | 19.619 | −5.676 | 1.00 | 0.80 | 1SG | 684 |
| ATOM | 684 | C | ILE | 85 | −10.027 | 20.301 | −5.752 | 1.00 | 0.80 | 1SG | 685 |
| ATOM | 685 | O | ILE | 85 | −10.687 | 20.945 | −4.942 | 1.00 | 0.80 | 1SG | 686 |
| ATOM | 686 | N | LEU | 86 | −8.748 | 20.599 | −6.036 | 1.00 | 0.68 | 1SG | 687 |
| ATOM | 687 | CA | LEU | 86 | −8.109 | 21.705 | −5.390 | 1.00 | 0.68 | 1SG | 688 |
| ATOM | 688 | CB | LEU | 86 | −6.651 | 21.905 | −5.847 | 1.00 | 0.68 | 1SG | 689 |
| ATOM | 689 | CG | LEU | 86 | −5.675 | 20.815 | −5.366 | 1.00 | 0.68 | 1SG | 690 |
| ATOM | 690 | CD2 | LEU | 86 | −5.713 | 20.672 | −3.836 | 1.00 | 0.68 | 1SG | 691 |
| ATOM | 691 | CD1 | LEU | 86 | −4.251 | 21.088 | −5.873 | 1.00 | 0.68 | 1SG | 692 |
| ATOM | 692 | C | LEU | 86 | −8.868 | 22.921 | −5.782 | 1.00 | 0.68 | 1SG | 693 |
| ATOM | 693 | O | LEU | 86 | −9.162 | 23.775 | −4.945 | 1.00 | 0.68 | 1SG | 694 |
| ATOM | 694 | N | ASN | 87 | −9.204 | 23.007 | −7.082 | 1.00 | 0.57 | 1SG | 695 |
| ATOM | 695 | CA | ASN | 87 | −9.905 | 24.121 | −7.643 | 1.00 | 0.57 | 1SG | 696 |
| ATOM | 696 | CB | ASN | 87 | −10.994 | 24.735 | −6.734 | 1.00 | 0.57 | 1SG | 697 |
| ATOM | 697 | CG | ASN | 87 | −11.906 | 25.612 | −7.581 | 1.00 | 0.57 | 1SG | 698 |
| ATOM | 698 | OD1 | ASN | 87 | −11.694 | 25.774 | −8.782 | 1.00 | 0.57 | 1SG | 699 |
| ATOM | 699 | ND2 | ASN | 87 | −12.949 | 26.204 | −6.938 | 1.00 | 0.57 | 1SG | 700 |
| ATOM | 700 | C | ASN | 87 | −8.856 | 25.137 | −7.939 | 1.00 | 0.57 | 1SG | 701 |
| ATOM | 701 | O | ASN | 87 | −7.715 | 24.789 | −8.241 | 1.00 | 0.57 | 1SG | 702 |
| ATOM | 702 | N | ASN | 88 | −9.213 | 26.427 | −7.860 | 1.00 | 0.71 | 1SG | 703 |
| ATOM | 703 | CA | ASN | 88 | −8.256 | 27.447 | −8.152 | 1.00 | 0.71 | 1SG | 704 |
| ATOM | 704 | CB | ASN | 88 | −8.916 | 28.752 | −8.625 | 1.00 | 0.71 | 1SG | 705 |
| ATOM | 705 | CG | ASN | 88 | −9.851 | 29.206 | −7.510 | 1.00 | 0.71 | 1SG | 706 |
| ATOM | 706 | OD1 | ASN | 88 | −10.756 | 28.475 | −7.110 | 1.00 | 0.71 | 1SG | 707 |
| ATOM | 707 | ND2 | ASN | 88 | −9.619 | 30.435 | −6.976 | 1.00 | 0.71 | 1SG | 708 |
| ATOM | 708 | C | ASN | 88 | −7.488 | 27.767 | −6.914 | 1.00 | 0.71 | 1SG | 709 |
| ATOM | 709 | O | ASN | 88 | −8.062 | 27.944 | −5.841 | 1.00 | 0.71 | 1SG | 710 |
| ATOM | 710 | N | LEU | 89 | −6.147 | 27.813 | −7.032 | 1.00 | 0.83 | 1SG | 711 |
| ATOM | 711 | CA | LEU | 89 | −5.346 | 28.284 | −5.945 | 1.00 | 0.83 | 1SG | 712 |
| ATOM | 712 | CB | LEU | 89 | −3.965 | 27.612 | −5.902 | 1.00 | 0.83 | 1SG | 713 |
| ATOM | 713 | CG | LEU | 89 | −3.074 | 28.087 | −4.746 | 1.00 | 0.83 | 1SG | 714 |
| ATOM | 714 | CD2 | LEU | 89 | −1.619 | 27.622 | −4.917 | 1.00 | 0.83 | 1SG | 715 |
| ATOM | 715 | CD1 | LEU | 89 | −3.695 | 27.698 | −3.392 | 1.00 | 0.83 | 1SG | 716 |
| ATOM | 716 | C | LEU | 89 | −5.118 | 29.708 | −6.298 | 1.00 | 0.83 | 1SG | 717 |
| ATOM | 717 | O | LEU | 89 | −3.984 | 30.138 | −6.505 | 1.00 | 0.83 | 1SG | 718 |
| ATOM | 718 | N | GLU | 90 | −6.214 | 30.485 | −6.329 | 1.00 | 0.67 | 1SG | 719 |
| ATOM | 719 | CA | GLU | 90 | −6.104 | 31.834 | −6.791 | 1.00 | 0.67 | 1SG | 720 |
| ATOM | 720 | CB | GLU | 90 | −7.471 | 32.519 | −6.996 | 1.00 | 0.67 | 1SG | 721 |
| ATOM | 721 | CG | GLU | 90 | −7.414 | 33.813 | −7.817 | 1.00 | 0.67 | 1SG | 722 |
| ATOM | 722 | CD | GLU | 90 | −7.648 | 33.461 | −9.283 | 1.00 | 0.67 | 1SG | 723 |
| ATOM | 723 | OE1 | GLU | 90 | −8.484 | 32.555 | −9.542 | 1.00 | 0.67 | 1SG | 724 |
| ATOM | 724 | OE2 | GLU | 90 | −7.009 | 34.097 | −10.164 | 1.00 | 0.67 | 1SG | 725 |
| ATOM | 725 | C | GLU | 90 | −5.380 | 32.585 | −5.743 | 1.00 | 0.67 | 1SG | 726 |
| ATOM | 726 | O | GLU | 90 | −5.964 | 33.013 | −4.749 | 1.00 | 0.67 | 1SG | 727 |
| ATOM | 727 | N | VAL | 91 | −4.059 | 32.751 | −5.923 | 1.00 | 0.42 | 1SG | 728 |
| ATOM | 728 | CA | VAL | 91 | −3.347 | 33.465 | −4.917 | 1.00 | 0.42 | 1SG | 729 |
| ATOM | 729 | CB | VAL | 91 | −2.493 | 32.589 | −4.077 | 1.00 | 0.42 | 1SG | 730 |
| ATOM | 730 | CG1 | VAL | 91 | −1.776 | 33.490 | −3.062 | 1.00 | 0.42 | 1SG | 731 |
| ATOM | 731 | CG2 | VAL | 91 | −3.377 | 31.474 | −3.494 | 1.00 | 0.42 | 1SG | 732 |
| ATOM | 732 | C | VAL | 91 | −2.417 | 34.423 | −5.570 | 1.00 | 0.42 | 1SG | 733 |
| ATOM | 733 | O | VAL | 91 | −1.235 | 34.127 | −5.737 | 1.00 | 0.42 | 1SG | 734 |
| ATOM | 734 | N | LYS | 92 | −2.931 | 35.595 | −5.959 | 1.00 | 0.62 | 1SG | 735 |
| ATOM | 735 | CA | LYS | 92 | −2.049 | 36.608 | −6.436 | 1.00 | 0.62 | 1SG | 736 |
| ATOM | 736 | CB | LYS | 92 | −2.610 | 37.378 | −7.640 | 1.00 | 0.62 | 1SG | 737 |
| ATOM | 737 | CG | LYS | 92 | −2.792 | 36.521 | −8.894 | 1.00 | 0.62 | 1SG | 738 |
| ATOM | 738 | CD | LYS | 92 | −3.660 | 37.184 | −9.967 | 1.00 | 0.62 | 1SG | 739 |
| ATOM | 739 | CE | LYS | 92 | −3.870 | 36.326 | −11.218 | 1.00 | 0.62 | 1SG | 740 |
| ATOM | 740 | NZ | LYS | 92 | −4.721 | 37.054 | −12.184 | 1.00 | 0.62 | 1SG | 741 |
| ATOM | 741 | C | LYS | 92 | −2.035 | 37.547 | −5.289 | 1.00 | 0.62 | 1SG | 742 |
| ATOM | 742 | O | LYS | 92 | −2.392 | 38.715 | −5.415 | 1.00 | 0.62 | 1SG | 743 |
| ATOM | 743 | N | LYS | 93 | −1.604 | 37.052 | −4.118 | 1.00 | 0.96 | 1SG | 744 |
| ATOM | 744 | CA | LYS | 93 | −1.757 | 37.880 | −2.966 | 1.00 | 0.96 | 1SG | 745 |
| ATOM | 745 | CB | LYS | 93 | −3.021 | 37.505 | −2.185 | 1.00 | 0.96 | 1SG | 746 |
| ATOM | 746 | CG | LYS | 93 | −3.449 | 38.507 | −1.117 | 1.00 | 0.96 | 1SG | 747 |
| ATOM | 747 | CD | LYS | 93 | −4.848 | 38.210 | −0.569 | 1.00 | 0.96 | 1SG | 748 |
| ATOM | 748 | CE | LYS | 93 | −5.922 | 38.085 | −1.654 | 1.00 | 0.96 | 1SG | 749 |
| ATOM | 749 | NZ | LYS | 93 | −5.831 | 36.765 | −2.321 | 1.00 | 0.96 | 1SG | 750 |
| ATOM | 750 | C | LYS | 93 | −0.612 | 37.654 | −2.043 | 1.00 | 0.96 | 1SG | 751 |
| ATOM | 751 | O | LYS | 93 | 0.517 | 38.065 | −2.306 | 1.00 | 0.96 | 1SG | 752 |
| ATOM | 752 | N | LYS | 94 | −0.905 | 36.975 | −0.917 | 1.00 | 1.04 | 1SG | 753 |
| ATOM | 753 | CA | LYS | 94 | 0.097 | 36.799 | 0.083 | 1.00 | 1.04 | 1SG | 754 |
| ATOM | 754 | CB | LYS | 94 | −0.488 | 36.592 | 1.490 | 1.00 | 1.04 | 1SG | 755 |
| ATOM | 755 | CG | LYS | 94 | 0.444 | 37.157 | 2.561 | 1.00 | 1.04 | 1SG | 756 |
| ATOM | 756 | CD | LYS | 94 | −0.273 | 37.601 | 3.839 | 1.00 | 1.04 | 1SG | 757 |
| ATOM | 757 | CE | LYS | 94 | 0.526 | 38.624 | 4.653 | 1.00 | 1.04 | 1SG | 758 |
| ATOM | 758 | NZ | LYS | 94 | −0.273 | 39.099 | 5.804 | 1.00 | 1.04 | 1SG | 759 |
| ATOM | 759 | C | LYS | 94 | 0.988 | 35.682 | −0.333 | 1.00 | 1.04 | 1SG | 760 |
| ATOM | 760 | O | LYS | 94 | 0.653 | 34.505 | −0.209 | 1.00 | 1.04 | 1SG | 761 |
| ATOM | 761 | N | ARG | 95 | 2.150 | 36.084 | −0.885 | 1.00 | 0.92 | 1SG | 762 |
| ATOM | 762 | CA | ARG | 95 | 3.153 | 35.205 | −1.403 | 1.00 | 0.92 | 1SG | 763 |
| ATOM | 763 | CB | ARG | 95 | 4.324 | 35.980 | −2.049 | 1.00 | 0.92 | 1SG | 764 |
| ATOM | 764 | CG | ARG | 95 | 5.143 | 36.806 | −1.058 | 1.00 | 0.92 | 1SG | 765 |
| ATOM | 765 | CD | ARG | 95 | 5.520 | 38.208 | −1.545 | 1.00 | 0.92 | 1SG | 766 |
| ATOM | 766 | NE | ARG | 95 | 4.339 | 39.086 | −1.299 | 1.00 | 0.92 | 1SG | 767 |
| ATOM | 767 | CZ | ARG | 95 | 4.387 | 40.397 | −1.671 | 1.00 | 0.92 | 1SG | 768 |
| ATOM | 768 | NH1 | ARG | 95 | 5.523 | 40.905 | −2.227 | 1.00 | 0.92 | 1SG | 769 |
| ATOM | 769 | NH2 | ARG | 95 | 3.301 | 41.205 | −1.501 | 1.00 | 0.92 | 1SG | 770 |
| ATOM | 770 | C | ARG | 95 | 3.755 | 34.415 | −0.281 | 1.00 | 0.92 | 1SG | 771 |
| ATOM | 771 | O | ARG | 95 | 3.960 | 33.207 | −0.406 | 1.00 | 0.92 | 1SG | 772 |
| ATOM | 772 | N | GLU | 96 | 4.019 | 35.086 | 0.859 | 1.00 | 0.64 | 1SG | 773 |
| ATOM | 773 | CA | GLU | 96 | 4.810 | 34.517 | 1.910 | 1.00 | 0.64 | 1SG | 774 |
| ATOM | 774 | CB | GLU | 96 | 5.066 | 35.489 | 3.073 | 1.00 | 0.64 | 1SG | 775 |
| ATOM | 775 | CG | GLU | 96 | 6.077 | 36.587 | 2.760 | 1.00 | 0.64 | 1SG | 776 |
| ATOM | 776 | CD | GLU | 96 | 6.540 | 37.123 | 4.101 | 1.00 | 0.64 | 1SG | 777 |
| ATOM | 777 | OE1 | GLU | 96 | 6.022 | 36.663 | 5.154 | 1.00 | 0.64 | 1SG | 778 |
| ATOM | 778 | OE2 | GLU | 96 | 7.437 | 38.003 | 4.091 | 1.00 | 0.64 | 1SG | 779 |
| ATOM | 779 | C | GLU | 96 | 4.228 | 33.270 | 2.480 | 1.00 | 0.64 | 1SG | 780 |
| ATOM | 780 | O | GLU | 96 | 4.934 | 32.264 | 2.571 | 1.00 | 0.64 | 1SG | 781 |
| ATOM | 781 | N | PRO | 97 | 2.982 | 33.246 | 2.838 | 1.00 | 0.57 | 1SG | 782 |
| ATOM | 782 | CA | PRO | 97 | 2.517 | 32.030 | 3.421 | 1.00 | 0.57 | 1SG | 783 |
| ATOM | 783 | CD | PRO | 97 | 2.272 | 34.402 | 3.362 | 1.00 | 0.57 | 1SG | 784 |
| ATOM | 784 | CB | PRO | 97 | 1.187 | 32.366 | 4.098 | 1.00 | 0.57 | 1SG | 785 |
| ATOM | 785 | CG | PRO | 97 | 0.894 | 33.827 | 3.708 | 1.00 | 0.57 | 1SG | 786 |
| ATOM | 786 | C | PRO | 97 | 2.493 | 31.050 | 2.316 | 1.00 | 0.57 | 1SG | 787 |
| ATOM | 787 | O | PRO | 97 | 2.509 | 31.491 | 1.170 | 1.00 | 0.57 | 1SG | 788 |
| ATOM | 788 | N | PHE | 98 | 2.541 | 29.740 | 2.620 | 1.00 | 0.69 | 1SG | 789 |
| ATOM | 789 | CA | PHE | 98 | 2.509 | 28.798 | 1.547 | 1.00 | 0.69 | 1SG | 790 |
| ATOM | 790 | CB | PHE | 98 | 2.931 | 27.382 | 1.938 | 1.00 | 0.69 | 1SG | 791 |
| ATOM | 791 | CG | PHE | 98 | 4.408 | 27.330 | 1.805 | 1.00 | 0.69 | 1SG | 792 |
| ATOM | 792 | CD1 | PHE | 98 | 4.962 | 27.311 | 0.544 | 1.00 | 0.69 | 1SG | 793 |
| ATOM | 793 | CD2 | PHE | 98 | 5.223 | 27.280 | 2.911 | 1.00 | 0.69 | 1SG | 794 |
| ATOM | 794 | CE1 | PHE | 98 | 6.323 | 27.251 | 0.379 | 1.00 | 0.69 | 1SG | 795 |
| ATOM | 795 | CE2 | PHE | 98 | 6.587 | 27.220 | 2.757 | 1.00 | 0.69 | 1SG | 796 |
| ATOM | 796 | CZ | PHE | 98 | 7.127 | 27.205 | 1.493 | 1.00 | 0.69 | 1SG | 797 |
| ATOM | 797 | C | PHE | 98 | 1.129 | 28.741 | 1.002 | 1.00 | 0.69 | 1SG | 798 |
| ATOM | 798 | O | PHE | 98 | 0.394 | 27.768 | 1.167 | 1.00 | 0.69 | 1SG | 799 |
| ATOM | 799 | N | THR | 99 | 0.775 | 29.800 | 0.260 | 1.00 | 0.72 | 1SG | 800 |
| ATOM | 800 | CA | THR | 99 | −0.502 | 29.941 | −0.338 | 1.00 | 0.72 | 1SG | 801 |
| ATOM | 801 | CB | THR | 99 | −0.669 | 31.276 | −0.979 | 1.00 | 0.72 | 1SG | 802 |
| ATOM | 802 | OG1 | THR | 99 | −2.049 | 31.553 | −1.092 | 1.00 | 0.72 | 1SG | 803 |
| ATOM | 803 | CG2 | THR | 99 | 0.004 | 31.263 | −2.360 | 1.00 | 0.72 | 1SG | 804 |
| ATOM | 804 | C | THR | 99 | −0.512 | 28.892 | −1.382 | 1.00 | 0.72 | 1SG | 805 |
| ATOM | 805 | O | THR | 99 | −1.556 | 28.430 | −1.831 | 1.00 | 0.72 | 1SG | 806 |
| ATOM | 806 | N | ASP | 100 | 0.703 | 28.456 | −1.756 | 1.00 | 0.88 | 1SG | 807 |
| ATOM | 807 | CA | ASP | 100 | 0.874 | 27.432 | −2.733 | 1.00 | 0.88 | 1SG | 808 |
| ATOM | 808 | CB | ASP | 100 | 2.362 | 27.029 | −2.849 | 1.00 | 0.88 | 1SG | 809 |
| ATOM | 809 | CG | ASP | 100 | 2.605 | 26.084 | −4.010 | 1.00 | 0.88 | 1SG | 810 |
| ATOM | 810 | OD1 | ASP | 100 | 1.849 | 25.083 | −4.145 | 1.00 | 0.88 | 1SG | 811 |
| ATOM | 811 | OD2 | ASP | 100 | 3.575 | 26.351 | −4.766 | 1.00 | 0.88 | 1SG | 812 |
| ATOM | 812 | C | ASP | 100 | 0.095 | 26.264 | −2.222 | 1.00 | 0.88 | 1SG | 813 |
| ATOM | 813 | O | ASP | 100 | −0.684 | 25.661 | −2.958 | 1.00 | 0.88 | 1SG | 814 |
| ATOM | 814 | N | THR | 101 | 0.258 | 25.923 | −0.932 | 1.00 | 1.17 | 1SG | 815 |
| ATOM | 815 | CA | THR | 101 | −0.549 | 24.856 | −0.410 | 1.00 | 1.17 | 1SG | 816 |
| ATOM | 816 | CB | THR | 101 | −0.092 | 23.480 | −0.842 | 1.00 | 1.17 | 1SG | 817 |
| ATOM | 817 | OG1 | THR | 101 | −1.056 | 22.487 | −0.520 | 1.00 | 1.17 | 1SG | 818 |
| ATOM | 818 | CG2 | THR | 101 | 1.258 | 23.181 | −0.174 | 1.00 | 1.17 | 1SG | 819 |
| ATOM | 819 | C | THR | 101 | −0.498 | 24.964 | 1.082 | 1.00 | 1.17 | 1SG | 820 |
| ATOM | 820 | O | THR | 101 | 0.543 | 25.297 | 1.638 | 1.00 | 1.17 | 1SG | 821 |
| ATOM | 821 | N | HIS | 102 | −1.655 | 24.726 | 1.744 | 1.00 | 1.24 | 1SG | 822 |
| ATOM | 822 | CA | HIS | 102 | −1.879 | 24.769 | 3.172 | 1.00 | 1.24 | 1SG | 823 |
| ATOM | 823 | ND1 | HIS | 102 | 1.382 | 25.854 | 5.068 | 1.00 | 1.24 | 1SG | 824 |
| ATOM | 824 | CG | HIS | 102 | 0.230 | 25.904 | 4.313 | 1.00 | 1.24 | 1SG | 825 |
| ATOM | 825 | CB | HIS | 102 | −0.610 | 24.672 | 4.055 | 1.00 | 1.24 | 1SG | 826 |
| ATOM | 826 | NE2 | HIS | 102 | 1.186 | 27.952 | 4.368 | 1.00 | 1.24 | 1SG | 827 |
| ATOM | 827 | CD2 | HIS | 102 | 0.122 | 27.199 | 3.893 | 1.00 | 1.24 | 1SG | 828 |
| ATOM | 828 | CE1 | HIS | 102 | 1.914 | 27.101 | 5.066 | 1.00 | 1.24 | 1SG | 829 |
| ATOM | 829 | C | HIS | 102 | −2.580 | 26.044 | 3.465 | 1.00 | 1.24 | 1SG | 830 |
| ATOM | 830 | O | HIS | 102 | −2.815 | 26.421 | 4.614 | 1.00 | 1.24 | 1SG | 831 |
| ATOM | 831 | N | TYR | 103 | −2.989 | 26.707 | 2.378 | 1.00 | 0.90 | 1SG | 832 |
| ATOM | 832 | CA | TYR | 103 | −3.585 | 27.996 | 2.499 | 1.00 | 0.90 | 1SG | 833 |
| ATOM | 833 | CB | TYR | 103 | −3.095 | 28.944 | 1.391 | 1.00 | 0.90 | 1SG | 834 |
| ATOM | 834 | CG | TYR | 103 | −3.657 | 30.271 | 1.723 | 1.00 | 0.90 | 1SG | 835 |
| ATOM | 835 | CD1 | TYR | 103 | −3.201 | 30.910 | 2.852 | 1.00 | 0.90 | 1SG | 836 |
| ATOM | 836 | CD2 | TYR | 103 | −4.608 | 30.865 | 0.924 | 1.00 | 0.90 | 1SG | 837 |
| ATOM | 837 | CE1 | TYR | 103 | −3.684 | 32.152 | 3.187 | 1.00 | 0.90 | 1SG | 838 |
| ATOM | 838 | CE2 | TYR | 103 | −5.092 | 32.108 | 1.255 | 1.00 | 0.90 | 1SG | 839 |
| ATOM | 839 | CZ | TYR | 103 | −4.631 | 32.753 | 2.385 | 1.00 | 0.90 | 1SG | 840 |
| ATOM | 840 | OH | TYR | 103 | −5.129 | 34.033 | 2.723 | 1.00 | 0.90 | 1SG | 841 |
| ATOM | 841 | C | TYR | 103 | −5.059 | 27.848 | 2.363 | 1.00 | 0.90 | 1SG | 842 |
| ATOM | 842 | O | TYR | 103 | −5.777 | 27.650 | 3.332 | 1.00 | 0.90 | 1SG | 843 |
| ATOM | 843 | N | LEU | 104 | −5.526 | 28.022 | 1.114 | 1.00 | 0.52 | 1SG | 844 |
| ATOM | 844 | CA | LEU | 104 | −6.922 | 27.891 | 0.844 | 1.00 | 0.52 | 1SG | 845 |
| ATOM | 845 | CB | LEU | 104 | −7.328 | 28.425 | −0.557 | 1.00 | 0.52 | 1SG | 846 |
| ATOM | 846 | CG | LEU | 104 | −7.220 | 29.956 | −0.742 | 1.00 | 0.52 | 1SG | 847 |
| ATOM | 847 | CD2 | LEU | 104 | −7.853 | 30.705 | 0.437 | 1.00 | 0.52 | 1SG | 848 |
| ATOM | 848 | CD1 | LEU | 104 | −7.820 | 30.413 | −2.079 | 1.00 | 0.52 | 1SG | 849 |
| ATOM | 849 | C | LEU | 104 | −7.289 | 26.428 | 0.880 | 1.00 | 0.52 | 1SG | 850 |
| ATOM | 850 | O | LEU | 104 | −8.252 | 26.007 | 1.515 | 1.00 | 0.52 | 1SG | 851 |
| ATOM | 851 | N | SER | 105 | −6.502 | 25.607 | 0.172 | 1.00 | 0.43 | 1SG | 852 |
| ATOM | 852 | CA | SER | 105 | −6.922 | 24.253 | −0.012 | 1.00 | 0.43 | 1SG | 853 |
| ATOM | 853 | CB | SER | 105 | −6.076 | 23.492 | −1.025 | 1.00 | 0.43 | 1SG | 854 |
| ATOM | 854 | OG | SER | 105 | −6.622 | 22.190 | −1.178 | 1.00 | 0.43 | 1SG | 855 |
| ATOM | 855 | C | SER | 105 | −6.911 | 23.430 | 1.241 | 1.00 | 0.43 | 1SG | 856 |
| ATOM | 856 | O | SER | 105 | −7.943 | 22.903 | 1.642 | 1.00 | 0.43 | 1SG | 857 |
| ATOM | 857 | N | LEU | 106 | −5.757 | 23.271 | 1.919 | 1.00 | 0.45 | 1SG | 858 |
| ATOM | 858 | CA | LEU | 106 | −5.748 | 22.384 | 3.059 | 1.00 | 0.45 | 1SG | 859 |
| ATOM | 859 | CB | LEU | 106 | −4.344 | 22.128 | 3.623 | 1.00 | 0.45 | 1SG | 860 |
| ATOM | 860 | CG | LEU | 106 | −3.406 | 21.430 | 2.618 | 1.00 | 0.45 | 1SG | 861 |
| ATOM | 861 | CD2 | LEU | 106 | −4.031 | 20.130 | 2.081 | 1.00 | 0.45 | 1SG | 862 |
| ATOM | 862 | CD1 | LEU | 106 | −2.015 | 21.210 | 3.240 | 1.00 | 0.45 | 1SG | 863 |
| ATOM | 863 | C | LEU | 106 | −6.580 | 22.946 | 4.150 | 1.00 | 0.45 | 1SG | 864 |
| ATOM | 864 | O | LEU | 106 | −7.448 | 22.275 | 4.703 | 1.00 | 0.45 | 1SG | 865 |
| ATOM | 865 | N | THR | 107 | −6.355 | 24.231 | 4.433 | 1.00 | 0.69 | 1SG | 866 |
| ATOM | 866 | CA | THR | 107 | −7.017 | 24.877 | 5.505 | 1.00 | 0.69 | 1SG | 867 |
| ATOM | 867 | CB | THR | 107 | −6.169 | 25.944 | 6.126 | 1.00 | 0.69 | 1SG | 868 |
| ATOM | 868 | OG1 | THR | 107 | −4.970 | 25.397 | 6.652 | 1.00 | 0.69 | 1SG | 869 |
| ATOM | 869 | CG2 | THR | 107 | −6.978 | 26.586 | 7.249 | 1.00 | 0.69 | 1SG | 870 |
| ATOM | 870 | C | THR | 107 | −8.237 | 25.530 | 4.968 | 1.00 | 0.69 | 1SG | 871 |
| ATOM | 871 | O | THR | 107 | −8.231 | 26.708 | 4.626 | 1.00 | 0.69 | 1SG | 872 |
| ATOM | 872 | N | ARG | 108 | −9.333 | 24.770 | 4.855 | 1.00 | 0.93 | 1SG | 873 |
| ATOM | 873 | CA | ARG | 108 | −10.548 | 25.409 | 4.459 | 1.00 | 0.93 | 1SG | 874 |
| ATOM | 874 | CB | ARG | 108 | −10.509 | 26.101 | 3.085 | 1.00 | 0.93 | 1SG | 875 |
| ATOM | 875 | CG | ARG | 108 | −11.896 | 26.566 | 2.640 | 1.00 | 0.93 | 1SG | 876 |
| ATOM | 876 | CD | ARG | 108 | −11.906 | 27.535 | 1.457 | 1.00 | 0.93 | 1SG | 877 |
| ATOM | 877 | NE | ARG | 108 | −13.278 | 27.479 | 0.876 | 1.00 | 0.93 | 1SG | 878 |
| ATOM | 878 | CZ | ARG | 108 | −14.327 | 28.145 | 1.446 | 1.00 | 0.93 | 1SG | 879 |
| ATOM | 879 | NH1 | ARG | 108 | −14.146 | 28.924 | 2.555 | 1.00 | 0.93 | 1SG | 880 |
| ATOM | 880 | NH2 | ARG | 108 | −15.578 | 27.977 | 0.930 | 1.00 | 0.93 | 1SG | 881 |
| ATOM | 881 | C | ARG | 108 | −11.656 | 24.416 | 4.480 | 1.00 | 0.93 | 1SG | 882 |
| ATOM | 882 | O | ARG | 108 | −11.563 | 23.341 | 3.889 | 1.00 | 0.93 | 1SG | 883 |
| ATOM | 883 | N | ASP | 109 | −12.737 | 24.764 | 5.204 | 1.00 | 0.83 | 1SG | 884 |
| ATOM | 884 | CA | ASP | 109 | −13.863 | 23.888 | 5.240 | 1.00 | 0.83 | 1SG | 885 |
| ATOM | 885 | CB | ASP | 109 | −14.941 | 24.320 | 6.244 | 1.00 | 0.83 | 1SG | 886 |
| ATOM | 886 | CG | ASP | 109 | −14.393 | 24.042 | 7.636 | 1.00 | 0.83 | 1SG | 887 |
| ATOM | 887 | OD1 | ASP | 109 | −13.817 | 22.938 | 7.829 | 1.00 | 0.83 | 1SG | 888 |
| ATOM | 888 | OD2 | ASP | 109 | −14.529 | 24.930 | 8.520 | 1.00 | 0.83 | 1SG | 889 |
| ATOM | 889 | C | ASP | 109 | −14.440 | 23.952 | 3.874 | 1.00 | 0.83 | 1SG | 890 |
| ATOM | 890 | O | ASP | 109 | −15.166 | 24.882 | 3.527 | 1.00 | 0.83 | 1SG | 891 |
| ATOM | 891 | N | CYS | 110 | −14.117 | 22.946 | 3.050 | 1.00 | 0.71 | 1SG | 892 |
| ATOM | 892 | CA | CYS | 110 | −14.578 | 23.002 | 1.705 | 1.00 | 0.71 | 1SG | 893 |
| ATOM | 893 | CB | CYS | 110 | −14.161 | 24.305 | 0.998 | 1.00 | 0.71 | 1SG | 894 |
| ATOM | 894 | SG | CYS | 110 | −15.106 | 24.620 | −0.520 | 1.00 | 0.71 | 1SG | 895 |
| ATOM | 895 | C | CYS | 110 | −13.921 | 21.855 | 1.019 | 1.00 | 0.71 | 1SG | 896 |
| ATOM | 896 | O | CYS | 110 | −14.280 | 20.699 | 1.237 | 1.00 | 0.71 | 1SG | 897 |
| ATOM | 897 | N | GLU | 111 | −12.925 | 22.155 | 0.169 | 1.00 | 0.73 | 1SG | 898 |
| ATOM | 898 | CA | GLU | 111 | −12.284 | 21.109 | −0.561 | 1.00 | 0.73 | 1SG | 899 |
| ATOM | 899 | CB | GLU | 111 | −11.138 | 21.649 | −1.435 | 1.00 | 0.73 | 1SG | 900 |
| ATOM | 900 | CG | GLU | 111 | −11.603 | 22.301 | −2.741 | 1.00 | 0.73 | 1SG | 901 |
| ATOM | 901 | CD | GLU | 111 | −12.601 | 23.410 | −2.449 | 1.00 | 0.73 | 1SG | 902 |
| ATOM | 902 | OE1 | GLU | 111 | −12.170 | 24.538 | −2.087 | 1.00 | 0.73 | 1SG | 903 |
| ATOM | 903 | OE2 | GLU | 111 | −13.822 | 23.130 | −2.593 | 1.00 | 0.73 | 1SG | 904 |
| ATOM | 904 | C | GLU | 111 | −11.721 | 20.081 | 0.375 | 1.00 | 0.73 | 1SG | 905 |
| ATOM | 905 | O | GLU | 111 | −12.255 | 18.976 | 0.468 | 1.00 | 0.73 | 1SG | 906 |
| ATOM | 906 | N | HIS | 112 | −10.635 | 20.402 | 1.113 | 1.00 | 0.76 | 1SG | 907 |
| ATOM | 907 | CA | HIS | 112 | −10.090 | 19.349 | 1.921 | 1.00 | 0.76 | 1SG | 908 |
| ATOM | 908 | ND1 | HIS | 112 | −7.748 | 16.972 | 1.839 | 1.00 | 0.76 | 1SG | 909 |
| ATOM | 909 | CG | HIS | 112 | −8.071 | 18.196 | 1.299 | 1.00 | 0.76 | 1SG | 910 |
| ATOM | 910 | CB | HIS | 112 | −8.572 | 19.343 | 2.110 | 1.00 | 0.76 | 1SG | 911 |
| ATOM | 911 | NE2 | HIS | 112 | −7.605 | 16.753 | −0.366 | 1.00 | 0.76 | 1SG | 912 |
| ATOM | 912 | CD2 | HIS | 112 | −7.970 | 18.048 | −0.048 | 1.00 | 0.76 | 1SG | 913 |
| ATOM | 913 | CE1 | HIS | 112 | −7.479 | 16.146 | 0.798 | 1.00 | 0.76 | 1SG | 914 |
| ATOM | 914 | C | HIS | 112 | −10.792 | 19.019 | 3.193 | 1.00 | 0.76 | 1SG | 915 |
| ATOM | 915 | O | HIS | 112 | −10.980 | 17.837 | 3.474 | 1.00 | 0.76 | 1SG | 916 |
| ATOM | 916 | N | PHE | 113 | −11.217 | 19.996 | 4.010 | 1.00 | 0.98 | 1SG | 917 |
| ATOM | 917 | CA | PHE | 113 | −11.829 | 19.508 | 5.214 | 1.00 | 0.98 | 1SG | 918 |
| ATOM | 918 | CB | PHE | 113 | −12.048 | 20.532 | 6.345 | 1.00 | 0.98 | 1SG | 919 |
| ATOM | 919 | CG | PHE | 113 | −10.844 | 20.470 | 7.227 | 1.00 | 0.98 | 1SG | 920 |
| ATOM | 920 | CD1 | PHE | 113 | −9.651 | 21.061 | 6.881 | 1.00 | 0.98 | 1SG | 921 |
| ATOM | 921 | CD2 | PHE | 113 | −10.935 | 19.791 | 8.421 | 1.00 | 0.98 | 1SG | 922 |
| ATOM | 922 | CE1 | PHE | 113 | −8.570 | 20.978 | 7.728 | 1.00 | 0.98 | 1SG | 923 |
| ATOM | 923 | CE2 | PHE | 113 | −9.857 | 19.699 | 9.265 | 1.00 | 0.98 | 1SG | 924 |
| ATOM | 924 | CZ | PHE | 113 | −8.673 | 20.297 | 8.916 | 1.00 | 0.98 | 1SG | 925 |
| ATOM | 925 | C | PHE | 113 | −13.101 | 18.791 | 4.910 | 1.00 | 0.98 | 1SG | 926 |
| ATOM | 926 | O | PHE | 113 | −13.677 | 18.925 | 3.832 | 1.00 | 0.98 | 1SG | 927 |
| ATOM | 927 | N | LYS | 114 | −13.530 | 18.006 | 5.920 | 1.00 | 1.10 | 1SG | 928 |
| ATOM | 928 | CA | LYS | 114 | −14.622 | 17.069 | 5.963 | 1.00 | 1.10 | 1SG | 929 |
| ATOM | 929 | CB | LYS | 114 | −15.572 | 17.241 | 7.173 | 1.00 | 1.10 | 1SG | 930 |
| ATOM | 930 | CG | LYS | 114 | −16.514 | 18.443 | 7.117 | 1.00 | 1.10 | 1SG | 931 |
| ATOM | 931 | CD | LYS | 114 | −15.830 | 19.796 | 6.961 | 1.00 | 1.10 | 1SG | 932 |
| ATOM | 932 | CE | LYS | 114 | −16.844 | 20.912 | 6.704 | 1.00 | 1.10 | 1SG | 933 |
| ATOM | 933 | NZ | LYS | 114 | −17.847 | 20.447 | 5.718 | 1.00 | 1.10 | 1SG | 934 |
| ATOM | 934 | C | LYS | 114 | −15.414 | 16.997 | 4.689 | 1.00 | 1.10 | 1SG | 935 |
| ATOM | 935 | O | LYS | 114 | −16.601 | 17.308 | 4.639 | 1.00 | 1.10 | 1SG | 936 |
| ATOM | 936 | N | ALA | 115 | −14.761 | 16.580 | 3.596 | 1.00 | 0.77 | 1SG | 937 |
| ATOM | 937 | CA | ALA | 115 | −15.474 | 16.389 | 2.371 | 1.00 | 0.77 | 1SG | 938 |
| ATOM | 938 | CB | ALA | 115 | −14.536 | 16.121 | 1.186 | 1.00 | 0.77 | 1SG | 939 |
| ATOM | 939 | C | ALA | 115 | −16.345 | 15.178 | 2.522 | 1.00 | 0.77 | 1SG | 940 |
| ATOM | 940 | O | ALA | 115 | −17.468 | 15.120 | 2.025 | 1.00 | 0.77 | 1SG | 941 |
| ATOM | 941 | N | GLU | 116 | −15.829 | 14.189 | 3.269 | 1.00 | 0.67 | 1SG | 942 |
| ATOM | 942 | CA | GLU | 116 | −16.407 | 12.883 | 3.418 | 1.00 | 0.67 | 1SG | 943 |
| ATOM | 943 | CB | GLU | 116 | −15.510 | 11.923 | 4.211 | 1.00 | 0.67 | 1SG | 944 |
| ATOM | 944 | CG | GLU | 116 | −14.313 | 11.413 | 3.405 | 1.00 | 0.67 | 1SG | 945 |
| ATOM | 945 | CD | GLU | 116 | −14.786 | 10.251 | 2.536 | 1.00 | 0.67 | 1SG | 946 |
| ATOM | 946 | OE1 | GLU | 116 | −15.586 | 10.494 | 1.595 | 1.00 | 0.67 | 1SG | 947 |
| ATOM | 947 | OE2 | GLU | 116 | −14.350 | 9.101 | 2.810 | 1.00 | 0.67 | 1SG | 948 |
| ATOM | 948 | C | GLU | 116 | −17.750 | 12.914 | 4.076 | 1.00 | 0.67 | 1SG | 949 |
| ATOM | 949 | O | GLU | 116 | −18.543 | 11.997 | 3.862 | 1.00 | 0.67 | 1SG | 950 |
| ATOM | 950 | N | ARG | 117 | −18.027 | 13.933 | 4.916 | 1.00 | 1.01 | 1SG | 951 |
| ATOM | 951 | CA | ARG | 117 | −19.265 | 13.981 | 5.649 | 1.00 | 1.01 | 1SG | 952 |
| ATOM | 952 | CB | ARG | 117 | −20.539 | 13.546 | 4.892 | 1.00 | 1.01 | 1SG | 953 |
| ATOM | 953 | CG | ARG | 117 | −21.401 | 14.649 | 4.278 | 1.00 | 1.01 | 1SG | 954 |
| ATOM | 954 | CD | ARG | 117 | −22.684 | 14.070 | 3.676 | 1.00 | 1.01 | 1SG | 955 |
| ATOM | 955 | NE | ARG | 117 | −23.733 | 15.129 | 3.634 | 1.00 | 1.01 | 1SG | 956 |
| ATOM | 956 | CZ | ARG | 117 | −25.044 | 14.753 | 3.715 | 1.00 | 1.01 | 1SG | 957 |
| ATOM | 957 | NH1 | ARG | 117 | −25.365 | 13.426 | 3.691 | 1.00 | 1.01 | 1SG | 958 |
| ATOM | 958 | NH2 | ARG | 117 | −26.026 | 15.691 | 3.855 | 1.00 | 1.01 | 1SG | 959 |
| ATOM | 959 | C | ARG | 117 | −19.120 | 13.017 | 6.763 | 1.00 | 1.01 | 1SG | 960 |
| ATOM | 960 | O | ARG | 117 | −18.406 | 13.270 | 7.732 | 1.00 | 1.01 | 1SG | 961 |
| ATOM | 961 | N | LYS | 118 | −19.799 | 11.862 | 6.648 | 1.00 | 1.37 | 1SG | 962 |
| ATOM | 962 | CA | LYS | 118 | −19.637 | 10.925 | 7.706 | 1.00 | 1.37 | 1SG | 963 |
| ATOM | 963 | CB | LYS | 118 | −20.817 | 10.830 | 8.694 | 1.00 | 1.37 | 1SG | 964 |
| ATOM | 964 | CG | LYS | 118 | −22.199 | 11.097 | 8.104 | 1.00 | 1.37 | 1SG | 965 |
| ATOM | 965 | CD | LYS | 118 | −22.740 | 10.030 | 7.155 | 1.00 | 1.37 | 1SG | 966 |
| ATOM | 966 | CE | LYS | 118 | −24.145 | 10.369 | 6.645 | 1.00 | 1.37 | 1SG | 967 |
| ATOM | 967 | NZ | LYS | 118 | −24.708 | 9.264 | 5.837 | 1.00 | 1.37 | 1SG | 968 |
| ATOM | 968 | C | LYS | 118 | −19.240 | 9.584 | 7.198 | 1.00 | 1.37 | 1SG | 969 |
| ATOM | 969 | O | LYS | 118 | −18.737 | 9.439 | 6.085 | 1.00 | 1.37 | 1SG | 970 |
| ATOM | 970 | N | PHE | 119 | −19.491 | 8.591 | 8.064 | 1.00 | 1.43 | 1SG | 971 |
| ATOM | 971 | CA | PHE | 119 | −18.981 | 7.254 | 8.119 | 1.00 | 1.43 | 1SG | 972 |
| ATOM | 972 | CB | PHE | 119 | −18.016 | 6.844 | 6.989 | 1.00 | 1.43 | 1SG | 973 |
| ATOM | 973 | CG | PHE | 119 | −18.783 | 6.310 | 5.819 | 1.00 | 1.43 | 1SG | 974 |
| ATOM | 974 | CD1 | PHE | 119 | −19.170 | 4.994 | 5.800 | 1.00 | 1 43 | 1SG | 975 |
| ATOM | 975 | CD2 | PHE | 119 | −19.100 | 7.088 | 4.728 | 1.00 | 1.43 | 1SG | 976 |
| ATOM | 976 | CE1 | PHE | 119 | −19.864 | 4.482 | 4.731 | 1.00 | 1.43 | 1SG | 977 |
| ATOM | 977 | CE2 | PHE | 119 | −19.793 | 6.597 | 3.649 | 1.00 | 1 43 | 1SG | 978 |
| ATOM | 978 | CZ | PHE | 119 | −20.179 | 5.284 | 3.659 | 1.00 | 1.43 | 1SG | 979 |
| ATOM | 979 | C | PHE | 119 | −18.210 | 7.483 | 9.369 | 1.00 | 1.43 | 1SG | 980 |
| ATOM | 980 | O | PHE | 119 | −17.057 | 7.075 | 9.523 | 1.00 | 1.43 | 1SG | 981 |
| ATOM | 981 | N | ILE | 120 | −18.970 | 8.138 | 10.287 | 1.00 | 1.15 | 1SG | 982 |
| ATOM | 982 | CA | ILE | 120 | −18.786 | 8.846 | 11.534 | 1.00 | 1.15 | 1SG | 983 |
| ATOM | 983 | CB | ILE | 120 | −20.015 | 8.672 | 12.411 | 1.00 | 1.15 | 1SG | 984 |
| ATOM | 984 | CG2 | ILE | 120 | −19.932 | 7.258 | 13.002 | 1.00 | 1.15 | 1SG | 985 |
| ATOM | 985 | CG1 | ILE | 120 | −20.154 | 9.746 | 13.511 | 1.00 | 1.15 | 1SG | 986 |
| ATOM | 986 | CD1 | ILE | 120 | −19.123 | 9.640 | 14.634 | 1.00 | 1.15 | 1SG | 987 |
| ATOM | 987 | C | ILE | 120 | −17.566 | 8.404 | 12.273 | 1.00 | 1.15 | 1SG | 988 |
| ATOM | 988 | O | ILE | 120 | −16.963 | 9.198 | 12.993 | 1.00 | 1 15 | 1SG | 989 |
| ATOM | 989 | N | GLN | 121 | −17.149 | 7.147 | 12.125 | 1.00 | 0.84 | 1SG | 990 |
| ATOM | 990 | CA | GLN | 121 | −15.999 | 6.737 | 12.862 | 1.00 | 0.84 | 1SG | 991 |
| ATOM | 991 | CB | GLN | 121 | −16.146 | 5.266 | 13.292 | 1.00 | 0.84 | 1SG | 992 |
| ATOM | 992 | CG | GLN | 121 | −15.228 | 4.778 | 14.418 | 1.00 | 0.84 | 1SG | 993 |
| ATOM | 993 | CD | GLN | 121 | −15.657 | 3.343 | 14.697 | 1.00 | 0.84 | 1SG | 994 |
| ATOM | 994 | OE1 | GLN | 121 | −14.869 | 2.438 | 14.963 | 1.00 | 0.84 | 1SG | 995 |
| ATOM | 995 | NE2 | GLN | 121 | −16.993 | 3.114 | 14.632 | 1.00 | 0.84 | 1SG | 996 |
| ATOM | 996 | C | GLN | 121 | −14.801 | 6.881 | 11.969 | 1.00 | 0.84 | 1SG | 997 |
| ATOM | 997 | O | GLN | 121 | −14.146 | 5.893 | 11.649 | 1.00 | 0.84 | 1SG | 998 |
| ATOM | 998 | N | PHE | 122 | −14.488 | 8.131 | 11.548 | 1.00 | 0.86 | 1SG | 999 |
| ATOM | 999 | CA | PHE | 122 | −13.361 | 8.381 | 10.686 | 1.00 | 0.86 | 1SG | 1000 |
| ATOM | 1000 | CB | PHE | 122 | −13.457 | 9.642 | 9.804 | 1.00 | 0.86 | 1SG | 1001 |
| ATOM | 1001 | CG | PHE | 122 | −14.217 | 9.376 | 8.563 | 1.00 | 0.86 | 1SG | 1002 |
| ATOM | 1002 | CD1 | PHE | 122 | −13.562 | 8.870 | 7.471 | 1.00 | 0.86 | 1SG | 1003 |
| ATOM | 1003 | CD2 | PHE | 122 | −15.562 | 9.636 | 8.480 | 1.00 | 0.86 | 1SG | 1004 |
| ATOM | 1004 | CE1 | PHE | 122 | −14.253 | 8.621 | 6.311 | 1.00 | 0.86 | 1SG | 1005 |
| ATOM | 1005 | CE2 | PHE | 122 | −16.260 | 9.389 | 7.319 | 1.00 | 0.86 | 1SG | 1006 |
| ATOM | 1006 | CZ | PHE | 122 | −15.601 | 8.879 | 6.226 | 1.00 | 0.86 | 1SG | 1007 |
| ATOM | 1007 | C | PHE | 122 | −12.139 | 8.635 | 11.492 | 1.00 | 0.86 | 1SG | 1008 |
| ATOM | 1008 | O | PHE | 122 | −12.127 | 9.488 | 12.379 | 1.00 | 0.86 | 1SG | 1009 |
| ATOM | 1009 | N | PRO | 123 | −11.125 | 7.865 | 11.180 | 1.00 | 0.96 | 1SG | 1010 |
| ATOM | 1010 | CA | PRO | 123 | −9.831 | 8.079 | 11.772 | 1.00 | 0.96 | 1SG | 1011 |
| ATOM | 1011 | CD | PRO | 123 | −11.389 | 6.447 | 10.983 | 1.00 | 0.96 | 1SG | 1012 |
| ATOM | 1012 | CB | PRO | 123 | −9.221 | 6.694 | 11.992 | 1.00 | 0.96 | 1SG | 1013 |
| ATOM | 1013 | CG | PRO | 123 | −10.411 | 5.726 | 11.902 | 1.00 | 0.96 | 1SG | 1014 |
| ATOM | 1014 | C | PRO | 123 | −9.065 | 8.818 | 10.719 | 1.00 | 0.96 | 1SG | 1015 |
| ATOM | 1015 | O | PRO | 123 | −9.661 | 9.106 | 9.683 | 1.00 | 0.96 | 1SG | 1016 |
| ATOM | 1016 | N | LEU | 124 | −7.762 | 9.115 | 10.935 | 1.00 | 0.77 | 1SG | 1017 |
| ATOM | 1017 | CA | LEU | 124 | −7.014 | 9.779 | 9.902 | 1.00 | 0.77 | 1SG | 1018 |
| ATOM | 1018 | CB | LEU | 124 | −7.067 | 11.313 | 10.035 | 1.00 | 0.77 | 1SG | 1019 |
| ATOM | 1019 | CG | LEU | 124 | −8.485 | 11.878 | 9.785 | 1.00 | 0.77 | 1SG | 1020 |
| ATOM | 1020 | CD2 | LEU | 124 | −9.030 | 11.432 | 8.414 | 1.00 | 0.77 | 1SG | 1021 |
| ATOM | 1021 | CD1 | LEU | 124 | −8.518 | 13.407 | 9.958 | 1.00 | 0.77 | 1SG | 1022 |
| ATOM | 1022 | C | LEU | 124 | −5.582 | 9.311 | 9.958 | 1.00 | 0.77 | 1SG | 1023 |
| ATOM | 1023 | O | LEU | 124 | −4.985 | 9.221 | 11.031 | 1.00 | 0.77 | 1SG | 1024 |
| ATOM | 1024 | N | SER | 125 | −4.992 | 9.001 | 8.780 | 1.00 | 0.60 | 1SG | 1025 |
| ATOM | 1025 | CA | SER | 125 | −3.648 | 8.492 | 8.736 | 1.00 | 0.60 | 1SG | 1026 |
| ATOM | 1026 | CB | SER | 125 | −3.558 | 7.065 | 8.168 | 1.00 | 0.60 | 1SG | 1027 |
| ATOM | 1027 | OG | SER | 125 | −4.267 | 6.149 | 8.989 | 1.00 | 0.60 | 1SG | 1028 |
| ATOM | 1028 | C | SER | 125 | −2.841 | 9.349 | 7.817 | 1.00 | 0.60 | 1SG | 1029 |
| ATOM | 1029 | O | SER | 125 | −3.365 | 9.951 | 6.881 | 1.00 | 0.60 | 1SG | 1030 |
| ATOM | 1030 | N | LYS | 126 | −1.522 | 9.425 | 8.077 | 1.00 | 0.60 | 1SG | 1031 |
| ATOM | 1031 | CA | LYS | 126 | −0.667 | 10.201 | 7.240 | 1.00 | 0.60 | 1SG | 1032 |
| ATOM | 1032 | CB | LYS | 126 | 0.347 | 11.040 | 8.041 | 1.00 | 0.60 | 1SG | 1033 |
| ATOM | 1033 | CG | LYS | 126 | 1.321 | 10.199 | 8.874 | 1.00 | 0.60 | 1SG | 1034 |
| ATOM | 1034 | CD | LYS | 126 | 2.352 | 11.008 | 9.674 | 1.00 | 0.60 | 1SG | 1035 |
| ATOM | 1035 | CE | LYS | 126 | 3.329 | 11.820 | 8.823 | 1.00 | 0.60 | 1SG | 1036 |
| ATOM | 1036 | NZ | LYS | 126 | 4.297 | 12.540 | 9.683 | 1.00 | 0.60 | 1SG | 1037 |
| ATOM | 1037 | C | LYS | 126 | 0.068 | 9.246 | 6.350 | 1.00 | 0.60 | 1SG | 1038 |
| ATOM | 1038 | O | LYS | 126 | 0.321 | 8.094 | 6.698 | 1.00 | 0.60 | 1SG | 1039 |
| ATOM | 1039 | N | GLU | 127 | 0.392 | 9.742 | 5.149 | 1.00 | 0.50 | 1SG | 1040 |
| ATOM | 1040 | CA | GLU | 127 | 1.082 | 9.066 | 4.093 | 1.00 | 0.50 | 1SG | 1041 |
| ATOM | 1041 | CB | GLU | 127 | 1.211 | 10.015 | 2.902 | 1.00 | 0.50 | 1SG | 1042 |
| ATOM | 1042 | CG | GLU | 127 | 1.682 | 11.373 | 3.434 | 1.00 | 0.50 | 1SG | 1043 |
| ATOM | 1043 | CD | GLU | 127 | 1.967 | 12.335 | 2.292 | 1.00 | 0.50 | 1SG | 1044 |
| ATOM | 1044 | OE1 | GLU | 127 | 3.007 | 12.129 | 1.615 | 1.00 | 0.50 | 1SG | 1045 |
| ATOM | 1045 | OE2 | GLU | 127 | 1.177 | 13.298 | 2.097 | 1.00 | 0.50 | 1SG | 1046 |
| ATOM | 1046 | C | GLU | 127 | 2.472 | 8.743 | 4.545 | 1.00 | 0.50 | 1SG | 1047 |
| ATOM | 1047 | O | GLU | 127 | 3.021 | 7.689 | 4.227 | 1.00 | 0.50 | 1SG | 1048 |
| ATOM | 1048 | N | GLU | 128 | 3.064 | 9.672 | 5.311 | 1.00 | 0.38 | 1SG | 1049 |
| ATOM | 1049 | CA | GLU | 128 | 4.432 | 9.630 | 5.726 | 1.00 | 0.38 | 1SG | 1050 |
| ATOM | 1050 | CB | GLU | 128 | 4.837 | 10.910 | 6.467 | 1.00 | 0.38 | 1SG | 1051 |
| ATOM | 1051 | CG | GLU | 128 | 4.795 | 12.150 | 5.572 | 1.00 | 0.38 | 1SG | 1052 |
| ATOM | 1052 | CD | GLU | 128 | 5.001 | 13.390 | 6.433 | 1.00 | 0.38 | 1SG | 1053 |
| ATOM | 1053 | OE1 | GLU | 128 | 4.012 | 13.834 | 7.078 | 1.00 | 0.38 | 1SG | 1054 |
| ATOM | 1054 | OE2 | GLU | 128 | 6.149 | 13.911 | 6.452 | 1.00 | 0.38 | 1SG | 1055 |
| ATOM | 1055 | C | GLU | 128 | 4.729 | 8.476 | 6.629 | 1.00 | 0.38 | 1SG | 1056 |
| ATOM | 1056 | O | GLU | 128 | 5.816 | 7.907 | 6.542 | 1.00 | 0.38 | 1SG | 1057 |
| ATOM | 1057 | N | VAL | 129 | 3.786 | 8.084 | 7.509 | 1.00 | 0.46 | 1SG | 1058 |
| ATOM | 1058 | CA | VAL | 129 | 4.130 | 7.107 | 8.508 | 1.00 | 0.46 | 1SG | 1059 |
| ATOM | 1059 | CB | VAL | 129 | 3.615 | 7.481 | 9.858 | 1.00 | 0.46 | 1SG | 1060 |
| ATOM | 1060 | CG1 | VAL | 129 | 4.258 | 8.825 | 10.236 | 1.00 | 0.46 | 1SG | 1061 |
| ATOM | 1061 | CG2 | VAL | 129 | 2.078 | 7.510 | 9.809 | 1.00 | 0.46 | 1SG | 1062 |
| ATOM | 1062 | C | VAL | 129 | 3.583 | 5.754 | 8.171 | 1.00 | 0.46 | 1SG | 1063 |
| ATOM | 1063 | O | VAL | 129 | 2.838 | 5.595 | 7.205 | 1.00 | 0.46 | 1SG | 1064 |
| ATOM | 1064 | N | GLU | 130 | 3.990 | 4.726 | 8.959 | 1.00 | 0.69 | 1SG | 1065 |
| ATOM | 1065 | CA | GLU | 130 | 3.548 | 3.382 | 8.701 | 1.00 | 0.69 | 1SG | 1066 |
| ATOM | 1066 | CB | GLU | 130 | 4.349 | 2.772 | 7.537 | 1.00 | 0.69 | 1SG | 1067 |
| ATOM | 1067 | CG | GLU | 130 | 3.824 | 1.463 | 6.960 | 1.00 | 0.69 | 1SG | 1068 |
| ATOM | 1068 | CD | GLU | 130 | 4.779 | 1.075 | 5.839 | 1.00 | 0.69 | 1SG | 1069 |
| ATOM | 1069 | OE1 | GLU | 130 | 6.012 | 1.245 | 6.034 | 1.00 | 0.69 | 1SG | 1070 |
| ATOM | 1070 | OE2 | GLU | 130 | 4.295 | 0.587 | 4.783 | 1.00 | 0.69 | 1SG | 1071 |
| ATOM | 1071 | C | GLU | 130 | 3.795 | 2.523 | 9.923 | 1.00 | 0.69 | 1SG | 1072 |
| ATOM | 1072 | O | GLU | 130 | 4.593 | 2.892 | 10.785 | 1.00 | 0.69 | 1SG | 1073 |
| ATOM | 1073 | N | PHE | 131 | 3.065 | 1.375 | 10.023 | 1.00 | 0.87 | 1SG | 1074 |
| ATOM | 1074 | CA | PHE | 131 | 3.257 | 0.308 | 10.985 | 1.00 | 0.87 | 1SG | 1075 |
| ATOM | 1075 | CB | PHE | 131 | 4.607 | 0.332 | 11.739 | 1.00 | 0.87 | 1SG | 1076 |
| ATOM | 1076 | CG | PHE | 131 | 5.685 | −0.049 | 10.779 | 1.00 | 0.87 | 1SG | 1077 |
| ATOM | 1077 | CD1 | PHE | 131 | 6.244 | 0.877 | 9.926 | 1.00 | 0.87 | 1SG | 1078 |
| ATOM | 1078 | CD2 | PHE | 131 | 6.141 | −1.345 | 10.740 | 1.00 | 0.87 | 1SG | 1079 |
| ATOM | 1079 | CE1 | PHE | 131 | 7.238 | 0.514 | 9.047 | 1.00 | 0.87 | 1SG | 1080 |
| ATOM | 1080 | CE2 | PHE | 131 | 7.134 | −1.713 | 9.864 | 1.00 | 0.87 | 1SG | 1081 |
| ATOM | 1081 | CZ | PHE | 131 | 7.684 | −0.784 | 9.014 | 1.00 | 0.87 | 1SG | 1082 |
| ATOM | 1082 | C | PHE | 131 | 2.069 | 0.008 | 11.889 | 1.00 | 0.87 | 1SG | 1083 |
| ATOM | 1083 | O | PHE | 131 | 1.044 | −0.443 | 11.381 | 1.00 | 0.87 | 1SG | 1084 |
| ATOM | 1084 | N | PRO | 132 | 2.121 | 0.200 | 13.195 | 1.00 | 0.90 | 1SG | 1085 |
| ATOM | 1085 | CA | PRO | 132 | 1.110 | −0.348 | 14.074 | 1.00 | 0.90 | 1SG | 1086 |
| ATOM | 1086 | CD | PRO | 132 | 2.696 | 1.409 | 13.776 | 1.00 | 0.90 | 1SG | 1087 |
| ATOM | 1087 | CB | PRO | 132 | 1.422 | 0.214 | 15.456 | 1.00 | 0.90 | 1SG | 1088 |
| ATOM | 1088 | CG | PRO | 132 | 2.002 | 1.598 | 15.135 | 1.00 | 0.90 | 1SG | 1089 |
| ATOM | 1089 | C | PRO | 132 | −0.333 | −0.139 | 13.738 | 1.00 | 0.90 | 1SG | 1090 |
| ATOM | 1090 | O | PRO | 132 | −0.898 | 0.875 | 14.148 | 1.00 | 0.90 | 1SG | 1091 |
| ATOM | 1091 | N | ILE | 133 | −0.956 | −1.075 | 12.995 | 1.00 | 0.81 | 1SG | 1092 |
| ATOM | 1092 | CA | ILE | 133 | −2.374 | −1.002 | 12.801 | 1.00 | 0.81 | 1SG | 1093 |
| ATOM | 1093 | CB | ILE | 133 | −2.829 | −1.678 | 11.531 | 1.00 | 0.81 | 1SG | 1094 |
| ATOM | 1094 | CG2 | ILE | 133 | −2.076 | −1.000 | 10.375 | 1.00 | 0.81 | 1SG | 1095 |
| ATOM | 1095 | CG1 | ILE | 133 | −2.636 | −3.206 | 11.554 | 1.00 | 0.81 | 1SG | 1096 |
| ATOM | 1096 | CD1 | ILE | 133 | −3.777 | −3.971 | 12.230 | 1.00 | 0.81 | 1SG | 1097 |
| ATOM | 1097 | C | ILE | 133 | −3.010 | −1.686 | 13.973 | 1.00 | 0.81 | 1SG | 1098 |
| ATOM | 1098 | O | ILE | 133 | −4.002 | −1.223 | 14.536 | 1.00 | 0.81 | 1SG | 1099 |
| ATOM | 1099 | N | ALA | 134 | −2.387 | −2.810 | 14.381 | 1.00 | 0.62 | 1SG | 1100 |
| ATOM | 1100 | CA | ALA | 134 | −2.862 | −3.715 | 15.386 | 1.00 | 0.62 | 1SG | 1101 |
| ATOM | 1101 | CB | ALA | 134 | −2.002 | −4.986 | 15.488 | 1.00 | 0.62 | 1SG | 1102 |
| ATOM | 1102 | C | ALA | 134 | −2.856 | −3.075 | 16.724 | 1.00 | 0.62 | 1SG | 1103 |
| ATOM | 1103 | O | ALA | 134 | −3.719 | −3.376 | 17.547 | 1.00 | 0.62 | 1SG | 1104 |
| ATOM | 1104 | N | TYR | 135 | −1.870 | −2.192 | 16.970 | 1.00 | 0.55 | 1SG | 1105 |
| ATOM | 1105 | CA | TYR | 135 | −1.690 | −1.601 | 18.260 | 1.00 | 0.55 | 1SG | 1106 |
| ATOM | 1106 | CB | TYR | 135 | −0.610 | −0.516 | 18.313 | 1.00 | 0.55 | 1SG | 1107 |
| ATOM | 1107 | CG | TYR | 135 | −0.698 | −0.077 | 19.718 | 1.00 | 0.55 | 1SG | 1108 |
| ATOM | 1108 | CD1 | TYR | 135 | −0.326 | −0.918 | 20.737 | 1.00 | 0.55 | 1SG | 1109 |
| ATOM | 1109 | CD2 | TYR | 135 | −1.175 | 1.172 | 20.006 | 1.00 | 0.55 | 1SG | 1110 |
| ATOM | 1110 | CE1 | TYR | 135 | −0.433 | −0.496 | 22.035 | 1.00 | 0.55 | 1SG | 1111 |
| ATOM | 1111 | CE2 | TYR | 135 | −1.282 | 1.599 | 21.305 | 1.00 | 0.55 | 1SG | 1112 |
| ATOM | 1112 | CZ | TYR | 135 | −0.907 | 0.757 | 22.322 | 1.00 | 0.55 | 1SG | 1113 |
| ATOM | 1113 | OH | TYR | 135 | −1.007 | 1.166 | 23.665 | 1.00 | 0.55 | 1SG | 1114 |
| ATOM | 1114 | C | TYR | 135 | −2.978 | −1.086 | 18.814 | 1.00 | 0.55 | 1SG | 1115 |
| ATOM | 1115 | O | TYR | 135 | −3.794 | −0.492 | 18.109 | 1.00 | 0.55 | 1SG | 1116 |
| ATOM | 1116 | N | SER | 136 | −3.179 | −1.353 | 20.121 | 1.00 | 0.67 | 1SG | 1117 |
| ATOM | 1117 | CA | SER | 136 | −4.391 | −1.047 | 20.813 | 1.00 | 0.67 | 1SG | 1118 |
| ATOM | 1118 | CB | SER | 136 | −4.466 | −1.694 | 22.206 | 1.00 | 0.67 | 1SG | 1119 |
| ATOM | 1119 | OG | SER | 136 | −3.479 | −1.135 | 23.060 | 1.00 | 0.67 | 1SG | 1120 |
| ATOM | 1120 | C | SER | 136 | −4.536 | 0.426 | 21.002 | 1.00 | 0.67 | 1SG | 1121 |
| ATOM | 1121 | O | SER | 136 | −5.117 | 0.866 | 21.993 | 1.00 | 0.67 | 1SG | 1122 |
| ATOM | 1122 | N | MET | 137 | −4.031 | 1.238 | 20.055 | 1.00 | 0.68 | 1SG | 1123 |
| ATOM | 1123 | CA | MET | 137 | −4.313 | 2.639 | 20.145 | 1.00 | 0.68 | 1SG | 1124 |
| ATOM | 1124 | CB | MET | 137 | −3.523 | 3.548 | 19.185 | 1.00 | 0.68 | 1SG | 1125 |
| ATOM | 1125 | CG | MET | 137 | −2.109 | 3.927 | 19.616 | 1.00 | 0.68 | 1SG | 1126 |
| ATOM | 1126 | SD | MET | 137 | −1.160 | 4.786 | 18.326 | 1.00 | 0.68 | 1SG | 1127 |
| ATOM | 1127 | CE | MET | 137 | −0.730 | 3.264 | 17.434 | 1.00 | 0.68 | 1SG | 1128 |
| ATOM | 1128 | C | MET | 137 | −5.707 | 2.741 | 19.666 | 1.00 | 0.68 | 1SG | 1129 |
| ATOM | 1129 | O | MET | 137 | −6.355 | 1.728 | 19.421 | 1.00 | 0.68 | 1SG | 1130 |
| ATOM | 1130 | N | VAL | 138 | −6.203 | 3.974 | 19.483 | 1.00 | 0.57 | 1SG | 1131 |
| ATOM | 1131 | CA | VAL | 138 | −7.533 | 4.083 | 18.983 | 1.00 | 0.57 | 1SG | 1132 |
| ATOM | 1132 | CB | VAL | 138 | −8.075 | 5.483 | 18.917 | 1.00 | 0.57 | 1SG | 1133 |
| ATOM | 1133 | CG1 | VAL | 138 | −9.428 | 5.477 | 18.190 | 1.00 | 0.57 | 1SG | 1134 |
| ATOM | 1134 | CG2 | VAL | 138 | −8.229 | 5.976 | 20.361 | 1.00 | 0.57 | 1SG | 1135 |
| ATOM | 1135 | C | VAL | 138 | −7.546 | 3.456 | 17.644 | 1.00 | 0.57 | 1SG | 1136 |
| ATOM | 1136 | O | VAL | 138 | −8.607 | 3.085 | 17.157 | 1.00 | 0.57 | 1SG | 1137 |
| ATOM | 1137 | N | ILE | 139 | −6.377 | 3.317 | 16.994 | 1.00 | 0.54 | 1SG | 1138 |
| ATOM | 1138 | CA | ILE | 139 | −6.462 | 2.724 | 15.705 | 1.00 | 0.54 | 1SG | 1139 |
| ATOM | 1139 | CB | ILE | 139 | −5.133 | 2.723 | 14.997 | 1.00 | 0.54 | 1SG | 1140 |
| ATOM | 1140 | CG2 | ILE | 139 | −4.089 | 2.017 | 15.878 | 1.00 | 0.54 | 1SG | 1141 |
| ATOM | 1141 | CG1 | ILE | 139 | −5.274 | 2.174 | 13.568 | 1.00 | 0.54 | 1SG | 1142 |
| ATOM | 1142 | CD1 | ILE | 139 | −4.038 | 2.437 | 12.710 | 1.00 | 0.54 | 1SG | 1143 |
| ATOM | 1143 | C | ILE | 139 | −7.024 | 1.331 | 15.808 | 1.00 | 0.54 | 1SG | 1144 |
| ATOM | 1144 | O | ILE | 139 | −8.158 | 1.107 | 15.391 | 1.00 | 0.54 | 1SG | 1145 |
| ATOM | 1145 | N | HIS | 140 | −6.300 | 0.352 | 16.383 | 1.00 | 0.59 | 1SG | 1146 |
| ATOM | 1146 | CA | HIS | 140 | −6.922 | −0.940 | 16.413 | 1.00 | 0.59 | 1SG | 1147 |
| ATOM | 1147 | ND1 | HIS | 140 | −7.025 | −4.060 | 17.635 | 1.00 | 0.59 | 1SG | 1148 |
| ATOM | 1148 | CG | HIS | 140 | −6.514 | −3.446 | 16.513 | 1.00 | 0.59 | 1SG | 1149 |
| ATOM | 1149 | CB | HIS | 140 | −5.896 | −2.079 | 16.510 | 1.00 | 0.59 | 1SG | 1150 |
| ATOM | 1150 | NE2 | HIS | 140 | −7.307 | −5.483 | 15.950 | 1.00 | 0.59 | 1SG | 1151 |
| ATOM | 1151 | CD2 | HIS | 140 | −6.694 | −4.329 | 15.493 | 1.00 | 0.59 | 1SG | 1152 |
| ATOM | 1152 | CE1 | HIS | 140 | −7.486 | −5.274 | 17.242 | 1.00 | 0.59 | 1SG | 1153 |
| ATOM | 1153 | C | HIS | 140 | −7.887 | −1.126 | 17.547 | 1.00 | 0.59 | 1SG | 1154 |
| ATOM | 1154 | O | HIS | 140 | −9.099 | −1.201 | 17.355 | 1.00 | 0.59 | 1SG | 1155 |
| ATOM | 1155 | N | GLU | 141 | −7.340 | −1.175 | 18.781 | 1.00 | 0.69 | 1SG | 1156 |
| ATOM | 1156 | CA | GLU | 141 | −8.110 | −1.550 | 19.936 | 1.00 | 0.69 | 1SG | 1157 |
| ATOM | 1157 | CB | GLU | 141 | −7.230 | −2.028 | 21.105 | 1.00 | 0.69 | 1SG | 1158 |
| ATOM | 1158 | CG | GLU | 141 | −8.004 | −2.855 | 22.133 | 1.00 | 0.69 | 1SG | 1159 |
| ATOM | 1159 | CD | GLU | 141 | −8.369 | −4.183 | 21.476 | 1.00 | 0.69 | 1SG | 1160 |
| ATOM | 1160 | OE1 | GLU | 141 | −9.436 | −4.236 | 20.807 | 1.00 | 0.69 | 1SG | 1161 |
| ATOM | 1161 | OE2 | GLU | 141 | −7.585 | −5.158 | 21.627 | 1.00 | 0.69 | 1SG | 1162 |
| ATOM | 1162 | C | GLU | 141 | −9.010 | −0.474 | 20.441 | 1.00 | 0.69 | 1SG | 1163 |
| ATOM | 1163 | O | GLU | 141 | −10.193 | −0.702 | 20.693 | 1.00 | 0.69 | 1SG | 1164 |
| ATOM | 1164 | N | LYS | 142 | −8.464 | 0.743 | 20.586 | 1.00 | 0.70 | 1SG | 1165 |
| ATOM | 1165 | CA | LYS | 142 | −9.182 | 1.818 | 21.198 | 1.00 | 0.70 | 1SG | 1166 |
| ATOM | 1166 | CB | LYS | 142 | −8.289 | 3.011 | 21.586 | 1.00 | 0.70 | 1SG | 1167 |
| ATOM | 1167 | CG | LYS | 142 | −8.891 | 4.016 | 22.571 | 1.00 | 0.70 | 1SG | 1168 |
| ATOM | 1168 | CD | LYS | 142 | −7.859 | 5.010 | 23.114 | 1.00 | 0.70 | 1SG | 1169 |
| ATOM | 1169 | CE | LYS | 142 | −8.392 | 5.922 | 24.221 | 1.00 | 0.70 | 1SG | 1170 |
| ATOM | 1170 | NZ | LYS | 142 | −9.408 | 6.848 | 23.695 | 1.00 | 0.70 | 1SG | 1171 |
| ATOM | 1171 | C | LYS | 142 | −10.329 | 2.243 | 20.336 | 1.00 | 0.70 | 1SG | 1172 |
| ATOM | 1172 | O | LYS | 142 | −11.334 | 2.730 | 20.847 | 1.00 | 0.70 | 1SG | 1173 |
| ATOM | 1173 | N | ILE | 143 | −10.225 | 2.157 | 18.998 | 1.00 | 0.64 | 1SG | 1174 |
| ATOM | 1174 | CA | ILE | 143 | −11.420 | 2.523 | 18.293 | 1.00 | 0.64 | 1SG | 1175 |
| ATOM | 1175 | CB | ILE | 143 | −11.277 | 2.746 | 16.808 | 1.00 | 0.64 | 1SG | 1176 |
| ATOM | 1176 | CG2 | ILE | 143 | −10.778 | 1.462 | 16.123 | 1.00 | 0.64 | 1SG | 1177 |
| ATOM | 1177 | CG1 | ILE | 143 | −12.590 | 3.297 | 16.237 | 1.00 | 0.64 | 1SG | 1178 |
| ATOM | 1178 | CD1 | ILE | 143 | −12.471 | 3.745 | 14.780 | 1.00 | 0.64 | 1SG | 1179 |
| ATOM | 1179 | C | ILE | 143 | −12.479 | 1.471 | 18.451 | 1.00 | 0.64 | 1SG | 1180 |
| ATOM | 1180 | O | ILE | 143 | −13.604 | 1.796 | 18.817 | 1.00 | 0 64 | 1SG | 1181 |
| ATOM | 1181 | N | GLU | 144 | −12.139 | 0.180 | 18.221 | 1.00 | 0.53 | 1SG | 1182 |
| ATOM | 1182 | CA | GLU | 144 | −13.142 | −0.849 | 18.124 | 1.00 | 0.53 | 1SG | 1183 |
| ATOM | 1183 | CB | GLU | 144 | −12.543 | −2.224 | 17.768 | 1.00 | 0.53 | 1SG | 1184 |
| ATOM | 1184 | CG | GLU | 144 | −13.585 | −3.311 | 17.476 | 1.00 | 0.53 | 1SG | 1185 |
| ATOM | 1185 | CD | GLU | 144 | −12.845 | −4.610 | 17.177 | 1.00 | 0.53 | 1SG | 1186 |
| ATOM | 1186 | OE1 | GLU | 144 | −11.641 | −4.692 | 17.539 | 1.00 | 0.53 | 1SG | 1187 |
| ATOM | 1187 | OE2 | GLU | 144 | −13.469 | −5.537 | 16.594 | 1.00 | 0.53 | 1SG | 1188 |
| ATOM | 1188 | C | GLU | 144 | −13.913 | −1.038 | 19.391 | 1.00 | 0.53 | 1SG | 1189 |
| ATOM | 1189 | O | GLU | 144 | −15.057 | −0.601 | 19.505 | 1.00 | 0.53 | 1SG | 1190 |
| ATOM | 1190 | N | ASN | 145 | −13.299 | −1.697 | 20.390 | 1.00 | 0.50 | 1SG | 1191 |
| ATOM | 1191 | CA | ASN | 145 | −13.997 | −1.988 | 21.609 | 1.00 | 0.50 | 1SG | 1192 |
| ATOM | 1192 | CB | ASN | 145 | −13.327 | −3.111 | 22.419 | 1.00 | 0.50 | 1SG | 1193 |
| ATOM | 1193 | CG | ASN | 145 | −13.538 | −4.401 | 21.638 | 1.00 | 0.50 | 1SG | 1194 |
| ATOM | 1194 | OD1 | ASN | 145 | −14.531 | −4.551 | 20.927 | 1.00 | 0.50 | 1SG | 1195 |
| ATOM | 1195 | ND2 | ASN | 145 | −12.584 | −5.361 | 21.768 | 1.00 | 0.50 | 1SG | 1196 |
| ATOM | 1196 | C | ASN | 145 | −14.015 | −0.753 | 22.433 | 1.00 | 0.50 | 1SG | 1197 |
| ATOM | 1197 | O | ASN | 145 | −14.990 | −0.442 | 23.115 | 1.00 | 0.50 | 1SG | 1198 |
| ATOM | 1198 | N | PHE | 146 | −12.899 | −0.018 | 22.361 | 1.00 | 0.71 | 1SG | 1199 |
| ATOM | 1199 | CA | PHE | 146 | −12.681 | 1.181 | 23.097 | 1.00 | 0.71 | 1SG | 1200 |
| ATOM | 1200 | CB | PHE | 146 | −11.206 | 1.535 | 23.306 | 1.00 | 0.71 | 1SG | 1201 |
| ATOM | 1201 | CG | PHE | 146 | −10.701 | 0.454 | 24.198 | 1.00 | 0.71 | 1SG | 1202 |
| ATOM | 1202 | CD1 | PHE | 146 | −10.867 | 0.539 | 25.562 | 1.00 | 0.71 | 1SG | 1203 |
| ATOM | 1203 | CD2 | PHE | 146 | −10.076 | −0.654 | 23.672 | 1.00 | 0.71 | 1SG | 1204 |
| ATOM | 1204 | CE1 | PHE | 146 | −10.412 | −0.460 | 26.390 | 1.00 | 0.71 | 1SG | 1205 |
| ATOM | 1205 | CE2 | PHE | 146 | −9.618 | −1.656 | 24.494 | 1.00 | 0.71 | 1SG | 1206 |
| ATOM | 1206 | CZ | PHE | 146 | −9.785 | −1.560 | 25.855 | 1.00 | 0.71 | 1SG | 1207 |
| ATOM | 1207 | C | PHE | 146 | −13.459 | 2.302 | 22.508 | 1.00 | 0.71 | 1SG | 1208 |
| ATOM | 1208 | O | PHE | 146 | −13.316 | 3.429 | 22.966 | 1.00 | 0.71 | 1SG | 1209 |
| ATOM | 1209 | N | GLU | 147 | −14.196 | 2.056 | 21.403 | 1.00 | 0.61 | 1SG | 1210 |
| ATOM | 1210 | CA | GLU | 147 | −15.010 | 3.096 | 20.831 | 1.00 | 0.61 | 1SG | 1211 |
| ATOM | 1211 | CB | GLU | 147 | −16.024 | 2.593 | 19.783 | 1.00 | 0.61 | 1SG | 1212 |
| ATOM | 1212 | CG | GLU | 147 | −17.045 | 1.594 | 20.335 | 1.00 | 0.61 | 1SG | 1213 |
| ATOM | 1213 | CD | GLU | 147 | −17.950 | 1.160 | 19.190 | 1.00 | 0.61 | 1SG | 1214 |
| ATOM | 1214 | OE1 | GLU | 147 | −17.440 | 1.052 | 18.043 | 1.00 | 0.61 | 1SG | 1215 |
| ATOM | 1215 | OE2 | GLU | 147 | −19.161 | 0.925 | 19.445 | 1.00 | 0.61 | 1SG | 1216 |
| ATOM | 1216 | C | GLU | 147 | −15.841 | 3.639 | 21.942 | 1.00 | 0.61 | 1SG | 1217 |
| ATOM | 1217 | O | GLU | 147 | −16.037 | 4.847 | 22.052 | 1.00 | 0.61 | 1SG | 1218 |
| ATOM | 1218 | N | ARG | 148 | −16.328 | 2.741 | 22.814 | 1.00 | 0.38 | 1SG | 1219 |
| ATOM | 1219 | CA | ARG | 148 | −17.138 | 3.158 | 23.913 | 1.00 | 0.38 | 1SG | 1220 |
| ATOM | 1220 | CB | ARG | 148 | −17.491 | 1.990 | 24.849 | 1.00 | 0.38 | 1SG | 1221 |
| ATOM | 1221 | CG | ARG | 148 | −18.208 | 0.831 | 24.149 | 1.00 | 0.38 | 1SG | 1222 |
| ATOM | 1222 | CD | ARG | 148 | −18.484 | −0.357 | 25.074 | 1.00 | 0.38 | 1SG | 1223 |
| ATOM | 1223 | NE | ARG | 148 | −18.893 | −1.518 | 24.232 | 1.00 | 0.38 | 1SG | 1224 |
| ATOM | 1224 | CZ | ARG | 148 | −20.202 | −1.697 | 23.890 | 1.00 | 0.38 | 1SG | 1225 |
| ATOM | 1225 | NH1 | ARG | 148 | −21.137 | −0.775 | 24.261 | 1.00 | 0.38 | 1SG | 1226 |
| ATOM | 1226 | NH2 | ARG | 148 | −20.577 | −2.805 | 23.187 | 1.00 | 0.38 | 1SG | 1227 |
| ATOM | 1227 | C | ARG | 148 | −16.307 | 4.123 | 24.690 | 1.00 | 0.38 | 1SG | 1228 |
| ATOM | 1228 | O | ARG | 148 | −16.776 | 5.167 | 25.128 | 1.00 | 0.38 | 1SG | 1229 |
| ATOM | 1229 | N | LEU | 149 | −15.021 | 3.805 | 24.882 | 1.00 | 0.40 | 1SG | 1230 |
| ATOM | 1230 | CA | LEU | 149 | −14.174 | 4.693 | 25.618 | 1.00 | 0.40 | 1SG | 1231 |
| ATOM | 1231 | CB | LEU | 149 | −12.777 | 4.087 | 25.846 | 1.00 | 0.40 | 1SG | 1232 |
| ATOM | 1232 | CG | LEU | 149 | −11.844 | 4.935 | 26.732 | 1.00 | 0.40 | 1SG | 1233 |
| ATOM | 1233 | CD2 | LEU | 149 | −12.509 | 5.238 | 28.085 | 1.00 | 0.40 | 1SG | 1234 |
| ATOM | 1234 | CD1 | LEU | 149 | −11.351 | 6.205 | 26.020 | 1.00 | 0.40 | 1SG | 1235 |
| ATOM | 1235 | C | LEU | 149 | −14.051 | 5.990 | 24.867 | 1.00 | 0.40 | 1SG | 1236 |
| ATOM | 1236 | O | LEU | 149 | −14.210 | 7.062 | 25.449 | 1.00 | 0.40 | 1SG | 1237 |
| ATOM | 1237 | N | LEU | 150 | −13.774 | 5.926 | 23.547 | 1.00 | 0.35 | 1SG | 1238 |
| ATOM | 1238 | CA | LEU | 150 | −13.589 | 7.118 | 22.772 | 1.00 | 0.35 | 1SG | 1239 |
| ATOM | 1239 | CB | LEU | 150 | −13.366 | 6.774 | 21.288 | 1.00 | 0.35 | 1SG | 1240 |
| ATOM | 1240 | CG | LEU | 150 | −13.241 | 7.984 | 20.344 | 1.00 | 0.35 | 1SG | 1241 |
| ATOM | 1241 | CD2 | LEU | 150 | −13.479 | 7.573 | 18.881 | 1.00 | 0.35 | 1SG | 1242 |
| ATOM | 1242 | CD1 | LEU | 150 | −11.921 | 8.739 | 20.558 | 1.00 | 0.35 | 1SG | 1243 |
| ATOM | 1243 | C | LEU | 150 | −14.833 | 7.949 | 22.816 | 1.00 | 0.35 | 1SG | 1244 |
| ATOM | 1244 | O | LEU | 150 | −14.854 | 9.009 | 23.440 | 1.00 | 0.35 | 1SG | 1245 |
| ATOM | 1245 | N | ARG | 151 | −15.907 | 7.478 | 22.146 | 1.00 | 0.28 | 1SG | 1246 |
| ATOM | 1246 | CA | ARG | 151 | −17.133 | 8.229 | 22.099 | 1.00 | 0.28 | 1SG | 1247 |
| ATOM | 1247 | CB | ARG | 151 | −18.069 | 7.874 | 20.928 | 1.00 | 0.28 | 1SG | 1248 |
| ATOM | 1248 | CG | ARG | 151 | −17.709 | 8.505 | 19.584 | 1.00 | 0.28 | 1SG | 1249 |
| ATOM | 1249 | CD | ARG | 151 | −18.946 | 8.906 | 18.768 | 1.00 | 0.28 | 1SG | 1250 |
| ATOM | 1250 | NE | ARG | 151 | −19.716 | 7.683 | 18.419 | 1.00 | 0.28 | 1SG | 1251 |
| ATOM | 1251 | CZ | ARG | 151 | −19.465 | 7.043 | 17.242 | 1.00 | 0.28 | 1SG | 1252 |
| ATOM | 1252 | NH1 | ARG | 151 | −18.464 | 7.491 | 16.429 | 1.00 | 0.28 | 1SG | 1253 |
| ATOM | 1253 | NH2 | ARG | 151 | −20.221 | 5.967 | 16.874 | 1.00 | 0.28 | 1SG | 1254 |
| ATOM | 1254 | C | ARG | 151 | −17.998 | 8.064 | 23.308 | 1.00 | 0.28 | 1SG | 1255 |
| ATOM | 1255 | O | ARG | 151 | −18.441 | 9.044 | 23.905 | 1.00 | 0.28 | 1SG | 1256 |
| ATOM | 1256 | N | ALA | 152 | −18.235 | 6.801 | 23.707 | 1.00 | 0.33 | 1SG | 1257 |
| ATOM | 1257 | CA | ALA | 152 | −19.273 | 6.484 | 24.651 | 1.00 | 0.33 | 1SG | 1258 |
| ATOM | 1258 | CB | ALA | 152 | −19.486 | 4.970 | 24.851 | 1.00 | 0.33 | 1SG | 1259 |
| ATOM | 1259 | C | ALA | 152 | −19.089 | 7.099 | 25.998 | 1.00 | 0.33 | 1SG | 1260 |
| ATOM | 1260 | O | ALA | 152 | −20.032 | 7.673 | 26.539 | 1.00 | 0.33 | 1SG | 1261 |
| ATOM | 1261 | N | VAL | 153 | −17.889 | 7.018 | 26.590 | 1.00 | 0.46 | 1SG | 1262 |
| ATOM | 1262 | CA | VAL | 153 | −17.769 | 7.548 | 27.913 | 1.00 | 0.46 | 1SG | 1263 |
| ATOM | 1263 | CB | VAL | 153 | −16.411 | 7.356 | 28.522 | 1.00 | 0.46 | 1SG | 1264 |
| ATOM | 1264 | CG1 | VAL | 153 | −15.405 | 8.203 | 27.723 | 1.00 | 0.46 | 1SG | 1265 |
| ATOM | 1265 | CG2 | VAL | 153 | −16.481 | 7.722 | 30.017 | 1.00 | 0.46 | 1SG | 1266 |
| ATOM | 1266 | C | VAL | 153 | −18.020 | 9.012 | 27.823 | 1.00 | 0.46 | 1SG | 1267 |
| ATOM | 1267 | O | VAL | 153 | −18.050 | 9.576 | 26.730 | 1.00 | 0.46 | 1SG | 1268 |
| ATOM | 1268 | N | TYR | 154 | −18.239 | 9.672 | 28.973 | 1.00 | 0.49 | 1SG | 1269 |
| ATOM | 1269 | CA | TYR | 154 | −18.430 | 11.085 | 28.896 | 1.00 | 0.49 | 1SG | 1270 |
| ATOM | 1270 | CB | TYR | 154 | −19.088 | 11.704 | 30.149 | 1.00 | 0.49 | 1SG | 1271 |
| ATOM | 1271 | CG | TYR | 154 | −20.554 | 11.408 | 30.178 | 1.00 | 0.49 | 1SG | 1272 |
| ATOM | 1272 | CD1 | TYR | 154 | −21.415 | 12.146 | 29.397 | 1.00 | 0.49 | 1SG | 1273 |
| ATOM | 1273 | CD2 | TYR | 154 | −21.078 | 10.426 | 30.991 | 1.00 | 0.49 | 1SG | 1274 |
| ATOM | 1274 | CE1 | TYR | 154 | −22.768 | 11.901 | 29.411 | 1.00 | 0.49 | 1SG | 1275 |
| ATOM | 1275 | CE2 | TYR | 154 | −22.434 | 10.176 | 31.010 | 1.00 | 0.49 | 1SG | 1276 |
| ATOM | 1276 | CZ | TYR | 154 | −23.283 | 10.914 | 30.216 | 1.00 | 0.49 | 1SG | 1277 |
| ATOM | 1277 | OH | TYR | 154 | −24.674 | 10.668 | 30.225 | 1.00 | 0.49 | 1SG | 1278 |
| ATOM | 1278 | C | TYR | 154 | −17.075 | 11.695 | 28.762 | 1.00 | 0.49 | 1SG | 1279 |
| ATOM | 1279 | O | TYR | 154 | −16.733 | 12.617 | 29.501 | 1.00 | 0.49 | 1SG | 1280 |
| ATOM | 1280 | N | ALA | 155 | −16.249 | 11.205 | 27.815 | 1.00 | 0.37 | 1SG | 1281 |
| ATOM | 1281 | CA | ALA | 155 | −14.998 | 11.879 | 27.672 | 1.00 | 0.37 | 1SG | 1282 |
| ATOM | 1282 | CB | ALA | 155 | −13.806 | 10.919 | 27.659 | 1.00 | 0.37 | 1SG | 1283 |
| ATOM | 1283 | C | ALA | 155 | −14.954 | 12.612 | 26.367 | 1.00 | 0.37 | 1SG | 1284 |
| ATOM | 1284 | O | ALA | 155 | −14.008 | 12.382 | 25.609 | 1.00 | 0.37 | 1SG | 1285 |
| ATOM | 1285 | N | PRO | 156 | −15.881 | 13.482 | 26.015 | 1.00 | 0.36 | 1SG | 1286 |
| ATOM | 1286 | CA | PRO | 156 | −15.639 | 14.188 | 24.804 | 1.00 | 0.36 | 1SG | 1287 |
| ATOM | 1287 | CD | PRO | 156 | −17.313 | 13.375 | 26.265 | 1.00 | 0.36 | 1SG | 1288 |
| ATOM | 1288 | CB | PRO | 156 | −16.979 | 14.701 | 24.279 | 1.00 | 0.36 | 1SG | 1289 |
| ATOM | 1289 | CG | PRO | 156 | −17.932 | 14.534 | 25.469 | 1.00 | 0.36 | 1SG | 1290 |
| ATOM | 1290 | C | PRO | 156 | −14.630 | 15.257 | 24.979 | 1.00 | 0.36 | 1SG | 1291 |
| ATOM | 1291 | O | PRO | 156 | −13.841 | 15.445 | 24.065 | 1.00 | 0.36 | 1SG | 1292 |
| ATOM | 1292 | N | GLN | 157 | −14.614 | 15.954 | 26.133 | 1.00 | 0.47 | 1SG | 1293 |
| ATOM | 1293 | CA | GLN | 157 | −13.701 | 17.056 | 26.258 | 1.00 | 0.47 | 1SG | 1294 |
| ATOM | 1294 | CB | GLN | 157 | −13.931 | 17.894 | 27.534 | 1.00 | 0.47 | 1SG | 1295 |
| ATOM | 1295 | CG | GLN | 157 | −15.219 | 18.714 | 27.512 | 1.00 | 0.47 | 1SG | 1296 |
| ATOM | 1296 | CD | GLN | 157 | −14.903 | 20.015 | 26.805 | 1.00 | 0.47 | 1SG | 1297 |
| ATOM | 1297 | OE1 | GLN | 157 | −15.753 | 20.597 | 26.132 | 1.00 | 0.47 | 1SG | 1298 |
| ATOM | 1298 | NE2 | GLN | 157 | −13.643 | 20.496 | 26.985 | 1.00 | 0.47 | 1SG | 1299 |
| ATOM | 1299 | C | GLN | 157 | −12.289 | 16.580 | 26.325 | 1.00 | 0.47 | 1SG | 1300 |
| ATOM | 1300 | O | GLN | 157 | −11.489 | 16.793 | 25.415 | 1.00 | 0.47 | 1SG | 1301 |
| ATOM | 1301 | N | ASN | 158 | −11.967 | 15.912 | 27.442 | 1.00 | 0.54 | 1SG | 1302 |
| ATOM | 1302 | CA | ASN | 158 | −10.639 | 15.479 | 27.741 | 1.00 | 0.54 | 1SG | 1303 |
| ATOM | 1303 | CB | ASN | 158 | −10.476 | 15.053 | 29.215 | 1.00 | 0.54 | 1SG | 1304 |
| ATOM | 1304 | CG | ASN | 158 | −11.503 | 13.973 | 29.546 | 1.00 | 0.54 | 1SG | 1305 |
| ATOM | 1305 | OD1 | ASN | 158 | −12.461 | 13.736 | 28.813 | 1.00 | 0.54 | 1SG | 1306 |
| ATOM | 1306 | ND2 | ASN | 158 | −11.316 | 13.306 | 30.716 | 1.00 | 0.54 | 1SG | 1307 |
| ATOM | 1307 | C | ASN | 158 | −10.182 | 14.393 | 26.825 | 1.00 | 0.54 | 1SG | 1308 |
| ATOM | 1308 | O | ASN | 158 | −9.062 | 14.464 | 26.325 | 1.00 | 0.54 | 1SG | 1309 |
| ATOM | 1309 | N | ILE | 159 | −11.015 | 13.368 | 26.554 | 1.00 | 0.67 | 1SG | 1310 |
| ATOM | 1310 | CA | ILE | 159 | −10.500 | 12.330 | 25.709 | 1.00 | 0.67 | 1SG | 1311 |
| ATOM | 1311 | CB | ILE | 159 | −11.029 | 10.965 | 26.033 | 1.00 | 0.67 | 1SG | 1312 |
| ATOM | 1312 | CG2 | ILE | 159 | −10.689 | 10.048 | 24.849 | 1.00 | 0.67 | 1SG | 1313 |
| ATOM | 1313 | CG1 | ILE | 159 | −10.480 | 10.465 | 27.376 | 1.00 | 0.67 | 1SG | 1314 |
| ATOM | 1314 | CD1 | ILE | 159 | −10.960 | 11.252 | 28.586 | 1.00 | 0.67 | 1SG | 1315 |
| ATOM | 1315 | C | ILE | 159 | −10.870 | 12.619 | 24.294 | 1.00 | 0.67 | 1SG | 1316 |
| ATOM | 1316 | O | ILE | 159 | −12.043 | 12.751 | 23.946 | 1.00 | 0.67 | 1SG | 1317 |
| ATOM | 1317 | N | TYR | 160 | −9.840 | 12.732 | 23.439 | 1.00 | 0.97 | 1SG | 1318 |
| ATOM | 1318 | CA | TYR | 160 | −10.073 | 12.970 | 22.051 | 1.00 | 0.97 | 1SG | 1319 |
| ATOM | 1319 | CB | TYR | 160 | −9.777 | 14.398 | 21.548 | 1.00 | 0.97 | 1SG | 1320 |
| ATOM | 1320 | CG | TYR | 160 | −10.994 | 15.238 | 21.646 | 1.00 | 0.97 | 1SG | 1321 |
| ATOM | 1321 | CD1 | TYR | 160 | −12.038 | 14.998 | 20.779 | 1.00 | 0.97 | 1SG | 1322 |
| ATOM | 1322 | CD2 | TYR | 160 | −11.083 | 16.280 | 22.535 | 1.00 | 0.97 | 1SG | 1323 |
| ATOM | 1323 | CE1 | TYR | 160 | −13.184 | 15.756 | 20.819 | 1.00 | 0.97 | 1SG | 1324 |
| ATOM | 1324 | CE2 | TYR | 160 | −12.224 | 17.047 | 22.581 | 1.00 | 0.97 | 1SG | 1325 |
| ATOM | 1325 | CZ | TYR | 160 | −13.278 | 16.785 | 21.729 | 1.00 | 0.97 | 1SG | 1326 |
| ATOM | 1326 | OH | TYR | 160 | −14.449 | 17.573 | 21.781 | 1.00 | 0.97 | 1SG | 1327 |
| ATOM | 1327 | C | TYR | 160 | −9.191 | 12.105 | 21.223 | 1.00 | 0.97 | 1SG | 1328 |
| ATOM | 1328 | O | TYR | 160 | −9.650 | 11.202 | 20.527 | 1.00 | 0.97 | 1SG | 1329 |
| ATOM | 1329 | N | CYS | 161 | −7.875 | 12.353 | 21.284 | 1.00 | 1.07 | 1SG | 1330 |
| ATOM | 1330 | CA | CYS | 161 | −7.027 | 11.744 | 20.306 | 1.00 | 1.07 | 1SG | 1331 |
| ATOM | 1331 | CB | CYS | 161 | −5.782 | 12.565 | 19.951 | 1.00 | 1.07 | 1SG | 1332 |
| ATOM | 1332 | SG | CYS | 161 | −5.948 | 14.333 | 20.286 | 1.00 | 1.07 | 1SG | 1333 |
| ATOM | 1333 | C | CYS | 161 | −6.345 | 10.579 | 20.918 | 1.00 | 1.07 | 1SG | 1334 |
| ATOM | 1334 | O | CYS | 161 | −6.564 | 10.208 | 22.070 | 1.00 | 1.07 | 1SG | 1335 |
| ATOM | 1335 | N | VAL | 162 | −5.513 | 9.972 | 20.052 | 1.00 | 0.82 | 1SG | 1336 |
| ATOM | 1336 | CA | VAL | 162 | −4.523 | 8.978 | 20.337 | 1.00 | 0.82 | 1SG | 1337 |
| ATOM | 1337 | CB | VAL | 162 | −5.024 | 7.571 | 20.276 | 1.00 | 0.82 | 1SG | 1338 |
| ATOM | 1338 | CG1 | VAL | 162 | −3.830 | 6.602 | 20.235 | 1.00 | 0.82 | 1SG | 1339 |
| ATOM | 1339 | CG2 | VAL | 162 | −5.903 | 7.359 | 21.520 | 1.00 | 0.82 | 1SG | 1340 |
| ATOM | 1340 | C | VAL | 162 | −3.485 | 9.209 | 19.282 | 1.00 | 0.82 | 1SG | 1341 |
| ATOM | 1341 | O | VAL | 162 | −3.834 | 9.356 | 18.113 | 1.00 | 0.82 | 1SG | 1342 |
| ATOM | 1342 | N | HIS | 163 | −2.187 | 9.268 | 19.657 | 1.00 | 0.78 | 1SG | 1343 |
| ATOM | 1343 | CA | HIS | 163 | −1.188 | 9.642 | 18.687 | 1.00 | 0.78 | 1SG | 1344 |
| ATOM | 1344 | ND1 | HIS | 163 | 0.733 | 12.570 | 17.477 | 1.00 | 0.78 | 1SG | 1345 |
| ATOM | 1345 | CG | HIS | 163 | −0.407 | 11.802 | 17.409 | 1.00 | 0.78 | 1SG | 1346 |
| ATOM | 1346 | CB | HIS | 163 | −1.100 | 11.177 | 18.584 | 1.00 | 0.78 | 1SG | 1347 |
| ATOM | 1347 | NE2 | HIS | 163 | 0.073 | 12.618 | 15.359 | 1.00 | 0.78 | 1SG | 1348 |
| ATOM | 1348 | CD2 | HIS | 163 | −0.790 | 11.839 | 16.106 | 1.00 | 0.78 | 1SG | 1349 |
| ATOM | 1349 | CE1 | HIS | 163 | 0.969 | 13.037 | 16.229 | 1.00 | 0.78 | 1SG | 1350 |
| ATOM | 1350 | C | HIS | 163 | 0.152 | 9.072 | 19.095 | 1.00 | 0.78 | 1SG | 1351 |
| ATOM | 1351 | O | HIS | 163 | 0.213 | 8.137 | 19.891 | 1.00 | 0.78 | 1SG | 1352 |
| ATOM | 1352 | N | VAL | 164 | 1.252 | 9.621 | 18.514 | 1.00 | 0.86 | 1SG | 1353 |
| ATOM | 1353 | CA | VAL | 164 | 2.644 | 9.241 | 18.693 | 1.00 | 0.86 | 1SG | 1354 |
| ATOM | 1354 | CB | VAL | 164 | 3.412 | 9.525 | 17.414 | 1.00 | 0.86 | 1SG | 1355 |
| ATOM | 1355 | CG1 | VAL | 164 | 2.569 | 10.523 | 16.631 | 1.00 | 0.86 | 1SG | 1356 |
| ATOM | 1356 | CG2 | VAL | 164 | 4.783 | 10.186 | 17.646 | 1.00 | 0.86 | 1SG | 1357 |
| ATOM | 1357 | C | VAL | 164 | 3.274 | 9.931 | 19.891 | 1.00 | 0.86 | 1SG | 1358 |
| ATOM | 1358 | O | VAL | 164 | 2.682 | 10.839 | 20.464 | 1.00 | 0.86 | 1SG | 1359 |
| ATOM | 1359 | N | ASP | 165 | 4.496 | 9.486 | 20.308 | 1.00 | 0.65 | 1SG | 1360 |
| ATOM | 1360 | CA | ASP | 165 | 5.281 | 9.965 | 21.425 | 1.00 | 0.65 | 1SG | 1361 |
| ATOM | 1361 | CB | ASP | 165 | 6.230 | 8.882 | 21.952 | 1.00 | 0.65 | 1SG | 1362 |
| ATOM | 1362 | CG | ASP | 165 | 6.847 | 9.339 | 23.265 | 1.00 | 0.65 | 1SG | 1363 |
| ATOM | 1363 | OD1 | ASP | 165 | 7.663 | 10.291 | 23.212 | 1.00 | 0.65 | 1SG | 1364 |
| ATOM | 1364 | OD2 | ASP | 165 | 6.535 | 8.746 | 24.331 | 1.00 | 0.65 | 1SG | 1365 |
| ATOM | 1365 | C | ASP | 165 | 6.171 | 11.113 | 21.022 | 1.00 | 0.65 | 1SG | 1366 |
| ATOM | 1366 | O | ASP | 165 | 6.876 | 11.054 | 20.015 | 1.00 | 0.65 | 1SG | 1367 |
| ATOM | 1367 | N | GLU | 166 | 6.167 | 12.192 | 21.838 | 1.00 | 0.65 | 1SG | 1368 |
| ATOM | 1368 | CA | GLU | 166 | 6.939 | 13.385 | 21.593 | 1.00 | 0.65 | 1SG | 1369 |
| ATOM | 1369 | CB | GLU | 166 | 6.655 | 14.470 | 22.649 | 1.00 | 0.65 | 1SG | 1370 |
| ATOM | 1370 | CG | GLU | 166 | 5.281 | 15.130 | 22.512 | 1.00 | 0.65 | 1SG | 1371 |
| ATOM | 1371 | CD | GLU | 166 | 5.509 | 16.483 | 21.854 | 1.00 | 0.65 | 1SG | 1372 |
| ATOM | 1372 | OE1 | GLU | 166 | 6.548 | 17.116 | 22.180 | 1.00 | 0.65 | 1SG | 1373 |
| ATOM | 1373 | OE2 | GLU | 166 | 4.656 | 16.913 | 21.033 | 1.00 | 0.65 | 1SG | 1374 |
| ATOM | 1374 | C | GLU | 166 | 8.410 | 13.107 | 21.650 | 1.00 | 0.65 | 1SG | 1375 |
| ATOM | 1375 | O | GLU | 166 | 9.137 | 13.378 | 20.695 | 1.00 | 0.65 | 1SG | 1376 |
| ATOM | 1376 | N | LYS | 167 | 8.906 | 12.544 | 22.766 | 1.00 | 0.87 | 1SG | 1377 |
| ATOM | 1377 | CA | LYS | 167 | 10.315 | 12.297 | 22.804 | 1.00 | 0.87 | 1SG | 1378 |
| ATOM | 1378 | CB | LYS | 167 | 11.107 | 13.084 | 23.857 | 1.00 | 0.87 | 1SG | 1379 |
| ATOM | 1379 | CG | LYS | 167 | 11.577 | 14.438 | 23.321 | 1.00 | 0.87 | 1SG | 1380 |
| ATOM | 1380 | CD | LYS | 167 | 12.484 | 14.302 | 22.090 | 1.00 | 0.87 | 1SG | 1381 |
| ATOM | 1331 | CE | LYS | 167 | 13.071 | 15.621 | 21.578 | 1.00 | 0.87 | 1SG | 1382 |
| ATOM | 1382 | NZ | LYS | 167 | 14.323 | 15.939 | 22.298 | 1.00 | 0.87 | 1SG | 1383 |
| ATOM | 1383 | C | LYS | 167 | 10.545 | 10.855 | 23.013 | 1.00 | 0.87 | 1SG | 1384 |
| ATOM | 1384 | O | LYS | 167 | 10.785 | 10.380 | 24.123 | 1.00 | 0.87 | 1SG | 1385 |
| ATOM | 1385 | N | SER | 168 | 10.540 | 10.114 | 21.898 | 1.00 | 0.78 | 1SG | 1386 |
| ATOM | 1386 | CA | SER | 168 | 10.787 | 8.715 | 21.979 | 1.00 | 0.78 | 1SG | 1387 |
| ATOM | 1387 | CB | SER | 168 | 9.717 | 7.874 | 21.261 | 1.00 | 0.78 | 1SG | 1388 |
| ATOM | 1388 | OG | SER | 168 | 9.618 | 8.267 | 19.900 | 1.00 | 0.78 | 1SG | 1389 |
| ATOM | 1389 | C | SER | 168 | 12.102 | 8.426 | 21.329 | 1.00 | 0.78 | 1SG | 1390 |
| ATOM | 1390 | O | SER | 168 | 12.135 | 7.847 | 20.245 | 1.00 | 0.78 | 1SG | 1391 |
| ATOM | 1391 | N | PRO | 169 | 13.211 | 8.794 | 21.910 | 1.00 | 0.54 | 1SG | 1392 |
| ATOM | 1392 | CA | PRO | 169 | 14.448 | 8.382 | 21.334 | 1.00 | 0.54 | 1SG | 1393 |
| ATOM | 1393 | CD | PRO | 169 | 13.384 | 9.981 | 22.717 | 1.00 | 0.54 | 1SG | 1394 |
| ATOM | 1394 | CB | PRO | 169 | 15.551 | 9.229 | 21.960 | 1.00 | 0.54 | 1SG | 1395 |
| ATOM | 1395 | CG | PRO | 169 | 14.857 | 9.919 | 23.133 | 1.00 | 0.54 | 1SG | 1396 |
| ATOM | 1396 | C | PRO | 169 | 14.508 | 6.957 | 21.715 | 1.00 | 0.54 | 1SG | 1397 |
| ATOM | 1397 | O | PRO | 169 | 13.865 | 6.577 | 22.693 | 1.00 | 0.54 | 1SG | 1398 |
| ATOM | 1398 | N | GLU | 170 | 15.255 | 6.146 | 20.966 | 1.00 | 0.53 | 1SG | 1399 |
| ATOM | 1399 | CA | GLU | 170 | 15.229 | 4.755 | 21.268 | 1.00 | 0.53 | 1SG | 1400 |
| ATOM | 1400 | CB | GLU | 170 | 16.024 | 3.941 | 20.232 | 1.00 | 0.53 | 1SG | 1401 |
| ATOM | 1401 | CG | GLU | 170 | 15.733 | 2.439 | 20.244 | 1.00 | 0.53 | 1SG | 1402 |
| ATOM | 1402 | CD | GLU | 170 | 16.938 | 1.725 | 20.832 | 1.00 | 0.53 | 1SG | 1403 |
| ATOM | 1403 | OE1 | GLU | 170 | 18.073 | 2.227 | 20.617 | 1.00 | 0.53 | 1SG | 1404 |
| ATOM | 1404 | OE2 | GLU | 170 | 16.750 | 0.664 | 21.483 | 1.00 | 0.53 | 1SG | 1405 |
| ATOM | 1405 | C | GLU | 170 | 15.770 | 4.566 | 22.649 | 1.00 | 0.53 | 1SG | 1406 |
| ATOM | 1406 | O | GLU | 170 | 15.247 | 3.770 | 23.428 | 1.00 | 0.53 | 1SG | 1407 |
| ATOM | 1407 | N | THR | 171 | 16.822 | 5.317 | 23.017 | 1.00 | 0.70 | 1SG | 1408 |
| ATOM | 1408 | CA | THR | 171 | 17.374 | 5.100 | 24.319 | 1.00 | 0.70 | 1SG | 1409 |
| ATOM | 1409 | CB | THR | 171 | 18.658 | 5.817 | 24.578 | 1.00 | 0.70 | 1SG | 1410 |
| ATOM | 1410 | OG1 | THR | 171 | 18.486 | 7.227 | 24.489 | 1.00 | 0.70 | 1SG | 1411 |
| ATOM | 1411 | CG2 | THR | 171 | 19.702 | 5.259 | 23.592 | 1.00 | 0.70 | 1SG | 1412 |
| ATOM | 1412 | C | THR | 171 | 16.420 | 5.495 | 25.393 | 1.00 | 0.70 | 1SG | 1413 |
| ATOM | 1413 | O | THR | 171 | 16.274 | 4.753 | 26.360 | 1.00 | 0.70 | 1SG | 1414 |
| ATOM | 1414 | N | PHE | 172 | 15.709 | 6.636 | 25.295 | 1.00 | 0.85 | 1SG | 1415 |
| ATOM | 1415 | CA | PHE | 172 | 14.995 | 6.883 | 26.510 | 1.00 | 0.85 | 1SG | 1416 |
| ATOM | 1416 | CB | PHE | 172 | 15.499 | 8.075 | 27.357 | 1.00 | 0.85 | 1SG | 1417 |
| ATOM | 1417 | CG | PHE | 172 | 15.667 | 9.348 | 26.610 | 1.00 | 0.85 | 1SG | 1418 |
| ATOM | 1418 | CD1 | PHE | 172 | 14.616 | 10.215 | 26.418 | 1.00 | 0.85 | 1SG | 1419 |
| ATOM | 1419 | CD2 | PHE | 172 | 16.912 | 9.685 | 26.131 | 1.00 | 0.85 | 1SG | 1420 |
| ATOM | 1420 | CE1 | PHE | 172 | 14.804 | 11.401 | 25.747 | 1.00 | 0.85 | 1SG | 1421 |
| ATOM | 1421 | CE2 | PHE | 172 | 17.108 | 10.865 | 25.453 | 1.00 | 0.85 | 1SG | 1422 |
| ATOM | 1422 | CZ | PHE | 172 | 16.049 | 11.721 | 25.262 | 1.00 | 0.85 | 1SG | 1423 |
| ATOM | 1423 | C | PHE | 172 | 13.507 | 6.802 | 26.443 | 1.00 | 0.85 | 1SG | 1424 |
| ATOM | 1424 | O | PHE | 172 | 12.795 | 7.761 | 26.733 | 1.00 | 0.85 | 1SG | 1425 |
| ATOM | 1425 | N | LYS | 173 | 13.018 | 5.596 | 26.098 | 1.00 | 0.88 | 1SG | 1426 |
| ATOM | 1426 | CA | LYS | 173 | 11.635 | 5.222 | 26.158 | 1.00 | 0.88 | 1SG | 1427 |
| ATOM | 1427 | CB | LYS | 173 | 10.971 | 5.092 | 24.776 | 1.00 | 0.88 | 1SG | 1428 |
| ATOM | 1428 | CG | LYS | 173 | 10.534 | 6.424 | 24.157 | 1.00 | 0.88 | 1SG | 1429 |
| ATOM | 1429 | CD | LYS | 173 | 9.348 | 7.078 | 24.875 | 1.00 | 0.88 | 1SG | 1430 |
| ATOM | 1430 | CE | LYS | 173 | 9.623 | 7.473 | 26.327 | 1.00 | 0.88 | 1SG | 1431 |
| ATOM | 1431 | NZ | LYS | 173 | 10.298 | 8.788 | 26.380 | 1.00 | 0.88 | 1SG | 1432 |
| ATOM | 1432 | C | LYS | 173 | 11.661 | 3.867 | 26.800 | 1.00 | 0.88 | 1SG | 1433 |
| ATOM | 1433 | O | LYS | 173 | 12.502 | 3.040 | 26.456 | 1.00 | 0.88 | 1SG | 1434 |
| ATOM | 1434 | N | GLU | 174 | 10.767 | 3.590 | 27.769 | 1.00 | 0.59 | 1SG | 1435 |
| ATOM | 1435 | CA | GLU | 174 | 10.864 | 2.300 | 28.388 | 1.00 | 0.59 | 1SG | 1436 |
| ATOM | 1436 | CB | GLU | 174 | 11.043 | 2.320 | 29.916 | 1.00 | 0.59 | 1SG | 1437 |
| ATOM | 1437 | CG | GLU | 174 | 11.237 | 0.909 | 30.472 | 1.00 | 0.59 | 1SG | 1438 |
| ATOM | 1438 | CD | GLU | 174 | 11.955 | 1.018 | 31.801 | 1.00 | 0.59 | 1SG | 1439 |
| ATOM | 1439 | OE1 | GLU | 174 | 13.100 | 1.541 | 31.807 | 1.00 | 0.59 | 1SG | 1440 |
| ATOM | 1440 | OE2 | GLU | 174 | 11.377 | 0.574 | 32.827 | 1.00 | 0.59 | 1SG | 1441 |
| ATOM | 1441 | C | GLU | 174 | 9.644 | 1.515 | 28.064 | 1.00 | 0.59 | 1SG | 1442 |
| ATOM | 1442 | O | GLU | 174 | 8.542 | 2.042 | 27.974 | 1.00 | 0.59 | 1SG | 1443 |
| ATOM | 1443 | N | ALA | 175 | 9.823 | 0.199 | 27.903 | 1.00 | 0.31 | 1SG | 1444 |
| ATOM | 1444 | CA | ALA | 175 | 8.771 | −0.674 | 27.485 | 1.00 | 0.31 | 1SG | 1445 |
| ATOM | 1445 | CB | ALA | 175 | 9.258 | −2.107 | 27.230 | 1.00 | 0.31 | 1SG | 1446 |
| ATOM | 1446 | C | ALA | 175 | 7.652 | −0.766 | 28.475 | 1.00 | 0.31 | 1SG | 1447 |
| ATOM | 1447 | O | ALA | 175 | 6.484 | −0.873 | 28.098 | 1.00 | 0.31 | 1SG | 1448 |
| ATOM | 1448 | N | VAL | 176 | 7.987 | −0.722 | 29.772 | 1.00 | 0.55 | 1SG | 1449 |
| ATOM | 1449 | CA | VAL | 176 | 7.042 | −1.050 | 30.798 | 1.00 | 0.55 | 1SG | 1450 |
| ATOM | 1450 | CB | VAL | 176 | 7.674 | −1.111 | 32.157 | 1.00 | 0.55 | 1SG | 1451 |
| ATOM | 1451 | CG1 | VAL | 176 | 6.590 | −1.437 | 33.201 | 1.00 | 0.55 | 1SG | 1452 |
| ATOM | 1452 | CG2 | VAL | 176 | 8.807 | −2.151 | 32.100 | 1.00 | 0.55 | 1SG | 1453 |
| ATOM | 1453 | C | VAL | 176 | 5.818 | −0.174 | 30.915 | 1.00 | 0.55 | 1SG | 1454 |
| ATOM | 1454 | O | VAL | 176 | 4.716 | −0.714 | 30.997 | 1.00 | 0.55 | 1SG | 1455 |
| ATOM | 1455 | N | LYS | 177 | 5.905 | 1.174 | 30.913 | 1.00 | 0.85 | 1SG | 1456 |
| ATOM | 1456 | CA | LYS | 177 | 4.683 | 1.816 | 31.337 | 1.00 | 0.85 | 1SG | 1457 |
| ATOM | 1457 | CB | LYS | 177 | 4.870 | 2.815 | 32.484 | 1.00 | 0.85 | 1SG | 1458 |
| ATOM | 1458 | CG | LYS | 177 | 3.536 | 3.268 | 33.078 | 1.00 | 0.85 | 1SG | 1459 |
| ATOM | 1459 | CD | LYS | 177 | 2.773 | 2.168 | 33.826 | 1.00 | 0.85 | 1SG | 1460 |
| ATOM | 1460 | CE | LYS | 177 | 1.985 | 1.218 | 32.920 | 1.00 | 0.85 | 1SG | 1461 |
| ATOM | 1461 | NZ | LYS | 177 | 1.175 | 0.294 | 33.746 | 1.00 | 0.85 | 1SG | 1462 |
| ATOM | 1462 | C | LYS | 177 | 3.894 | 2.507 | 30.268 | 1.00 | 0.85 | 1SG | 1463 |
| ATOM | 1463 | O | LYS | 177 | 4.366 | 3.394 | 29.566 | 1.00 | 0.85 | 1SG | 1464 |
| ATOM | 1464 | N | ALA | 178 | 2.589 | 2.191 | 30.267 | 1.00 | 0.89 | 1SG | 1465 |
| ATOM | 1465 | CA | ALA | 178 | 1.564 | 2.553 | 29.331 | 1.00 | 0.89 | 1SG | 1466 |
| ATOM | 1466 | CB | ALA | 178 | 0.212 | 1.911 | 29.694 | 1.00 | 0.89 | 1SG | 1467 |
| ATOM | 1467 | C | ALA | 178 | 1.331 | 4.034 | 29.225 | 1.00 | 0.89 | 1SG | 1468 |
| ATOM | 1468 | O | ALA | 178 | 0.819 | 4.468 | 28.196 | 1.00 | 0.89 | 1SG | 1469 |
| ATOM | 1469 | N | ILE | 179 | 1.667 | 4.825 | 30.270 | 1.00 | 0.99 | 1SG | 1470 |
| ATOM | 1470 | CA | ILE | 179 | 1.350 | 6.235 | 30.415 | 1.00 | 0.99 | 1SG | 1471 |
| ATOM | 1471 | CB | ILE | 179 | 2.256 | 7.028 | 31.330 | 1.00 | 0.99 | 1SG | 1472 |
| ATOM | 1472 | CG2 | ILE | 179 | 2.156 | 6.406 | 32.733 | 1.00 | 0.99 | 1SG | 1473 |
| ATOM | 1473 | CG1 | ILE | 179 | 3.699 | 7.115 | 30.804 | 1.00 | 0.99 | 1SG | 1474 |
| ATOM | 1474 | CD1 | ILE | 179 | 4.457 | 5.796 | 30.858 | 1.00 | 0.99 | 1SG | 1475 |
| ATOM | 1475 | C | ILE | 179 | 1.245 | 7.002 | 29.124 | 1.00 | 0.99 | 1SG | 1476 |
| ATOM | 1476 | O | ILE | 179 | 1.935 | 6.724 | 28.145 | 1.00 | 0.99 | 1SG | 1477 |
| ATOM | 1477 | N | ILE | 180 | 0.366 | 8.037 | 29.150 | 1.00 | 0.94 | 1SG | 1478 |
| ATOM | 1478 | CA | ILE | 180 | −0.052 | 8.884 | 28.055 | 1.00 | 0.94 | 1SG | 1479 |
| ATOM | 1479 | CB | ILE | 180 | −1.547 | 8.892 | 27.962 | 1.00 | 0.94 | 1SG | 1480 |
| ATOM | 1480 | CG2 | ILE | 180 | −1.989 | 7.436 | 27.733 | 1.00 | 0.94 | 1SG | 1481 |
| ATOM | 1481 | CG1 | ILE | 180 | −2.147 | 9.487 | 29.253 | 1.00 | 0.94 | 1SG | 1482 |
| ATOM | 1482 | CD1 | ILE | 180 | −3.667 | 9.620 | 29.233 | 1.00 | 0.94 | 1SG | 1483 |
| ATOM | 1483 | C | ILE | 180 | 0.411 | 10.308 | 28.334 | 1.00 | 0.94 | 1SG | 1484 |
| ATOM | 1484 | O | ILE | 180 | 1.161 | 10.517 | 29.286 | 1.00 | 0.94 | 1SG | 1485 |
| ATOM | 1485 | N | SER | 181 | 0.013 | 11.320 | 27.498 | 1.00 | 0.69 | 1SG | 1486 |
| ATOM | 1486 | CA | SER | 181 | 0.491 | 12.686 | 27.680 | 1.00 | 0.69 | 1SG | 1487 |
| ATOM | 1487 | CB | SER | 181 | 1.580 | 13.111 | 26.678 | 1.00 | 0.69 | 1SG | 1488 |
| ATOM | 1488 | OG | SER | 181 | 2.799 | 12.449 | 26.953 | 1.00 | 0.69 | 1SG | 1489 |
| ATOM | 1489 | C | SER | 181 | −0.587 | 13.737 | 27.531 | 1.00 | 0.69 | 1SG | 1490 |
| ATOM | 1490 | O | SER | 181 | −1.555 | 13.569 | 26.791 | 1.00 | 0.69 | 1SG | 1491 |
| ATOM | 1491 | N | CYS | 182 | −0.417 | 14.858 | 28.282 | 1.00 | 0.46 | 1SG | 1492 |
| ATOM | 1492 | CA | CYS | 182 | −1.256 | 16.035 | 28.285 | 1.00 | 0.46 | 1SG | 1493 |
| ATOM | 1493 | CB | CYS | 182 | −0.966 | 16.974 | 29.464 | 1.00 | 0.46 | 1SG | 1494 |
| ATOM | 1494 | SG | CYS | 182 | −2.031 | 18.445 | 29.440 | 1.00 | 0.46 | 1SG | 1495 |
| ATOM | 1495 | C | CYS | 182 | −1.056 | 16.839 | 27.041 | 1.00 | 0.46 | 1SG | 1496 |
| ATOM | 1496 | O | CYS | 182 | −2.011 | 17.340 | 26.449 | 1.00 | 0.46 | 1SG | 1497 |
| ATOM | 1497 | N | PHE | 183 | 0.209 | 17.014 | 26.621 | 1.00 | 0.27 | 1SG | 1498 |
| ATOM | 1498 | CA | PHE | 183 | 0.432 | 17.771 | 25.430 | 1.00 | 0.27 | 1SG | 1499 |
| ATOM | 1499 | CB | PHE | 183 | 1.936 | 17.917 | 25.081 | 1.00 | 0.27 | 1SG | 1500 |
| ATOM | 1500 | CG | PHE | 183 | 2.119 | 18.927 | 23.996 | 1.00 | 0.27 | 1SG | 1501 |
| ATOM | 1501 | CD1 | PHE | 183 | 2.242 | 20.262 | 24.309 | 1.00 | 0.27 | 1SG | 1502 |
| ATOM | 1502 | CD2 | PHE | 183 | 2.166 | 18.557 | 22.670 | 1.00 | 0.27 | 1SG | 1503 |
| ATOM | 1503 | CE1 | PHE | 183 | 2.409 | 21.208 | 23.325 | 1.00 | 0.27 | 1SG | 1504 |
| ATOM | 1504 | CE2 | PHE | 183 | 2.332 | 19.499 | 21.680 | 1.00 | 0.27 | 1SG | 1505 |
| ATOM | 1505 | CZ | PHE | 183 | 2.455 | 20.829 | 22.005 | 1.00 | 0.27 | 1SG | 1506 |
| ATOM | 1506 | C | PHE | 183 | −0.232 | 16.940 | 24.400 | 1.00 | 0.27 | 1SG | 1507 |
| ATOM | 1507 | O | PHE | 183 | −0.140 | 15.716 | 24.495 | 1.00 | 0.27 | 1SG | 1508 |
| ATOM | 1508 | N | PRO | 184 | −0.942 | 17.561 | 23.480 | 1.00 | 0.22 | 1SG | 1509 |
| ATOM | 1509 | CA | PRO | 184 | −1.636 | 16.859 | 22.444 | 1.00 | 0.22 | 1SG | 1510 |
| ATOM | 1510 | CD | PRO | 184 | −0.789 | 18.975 | 23.176 | 1.00 | 0.22 | 1SG | 1511 |
| ATOM | 1511 | CB | PRO | 184 | −2.126 | 17.920 | 21.459 | 1.00 | 0.22 | 1SG | 1512 |
| ATOM | 1512 | CG | PRO | 184 | −1.173 | 19.106 | 21.693 | 1.00 | 0.22 | 1SG | 1513 |
| ATOM | 1513 | C | PRO | 184 | −0.673 | 15.901 | 21.858 | 1.00 | 0.22 | 1SG | 1514 |
| ATOM | 1514 | O | PRO | 184 | 0.406 | 16.312 | 21.431 | 1.00 | 0.22 | 1SG | 1515 |
| ATOM | 1515 | N | ASN | 185 | −1.086 | 14.625 | 21.813 | 1.00 | 0.20 | 1SG | 1516 |
| ATOM | 1516 | CA | ASN | 185 | −0.238 | 13.543 | 21.434 | 1.00 | 0.20 | 1SG | 1517 |
| ATOM | 1517 | CB | ASN | 185 | −0.999 | 12.226 | 21.317 | 1.00 | 0.20 | 1SG | 1518 |
| ATOM | 1518 | CG | ASN | 185 | −1.519 | 11.884 | 22.696 | 1.00 | 0.20 | 1SG | 1519 |
| ATOM | 1519 | OD1 | ASN | 185 | −0.839 | 12.073 | 23.703 | 1.00 | 0.20 | 1SG | 1520 |
| ATOM | 1520 | ND2 | ASN | 185 | −2.778 | 11.376 | 22.742 | 1.00 | 0.20 | 1SG | 1521 |
| ATOM | 1521 | C | ASN | 185 | 0.370 | 13.904 | 20.134 | 1.00 | 0.20 | 1SG | 1522 |
| ATOM | 1522 | O | ASN | 185 | 1.489 | 13.483 | 19.853 | 1.00 | 0.20 | 1SG | 1523 |
| ATOM | 1523 | N | VAL | 186 | −0.371 | 14.707 | 19.344 | 1.00 | 0.39 | 1SG | 1524 |
| ATOM | 1524 | CA | VAL | 186 | 0.093 | 15.288 | 18.127 | 1.00 | 0.39 | 1SG | 1525 |
| ATOM | 1525 | CB | VAL | 186 | −0.733 | 16.466 | 17.721 | 1.00 | 0.39 | 1SG | 1526 |
| ATOM | 1526 | CG1 | VAL | 186 | −0.187 | 17.038 | 16.406 | 1.00 | 0.39 | 1SG | 1527 |
| ATOM | 1527 | CG2 | VAL | 186 | −2.221 | 16.099 | 17.774 | 1.00 | 0.39 | 1SG | 1528 |
| ATOM | 1528 | C | VAL | 186 | 1.380 | 15.921 | 18.513 | 1.00 | 0.39 | 1SG | 1529 |
| ATOM | 1529 | O | VAL | 186 | 1.380 | 17.016 | 19.074 | 1.00 | 0.39 | 1SG | 1530 |
| ATOM | 1530 | N | PHE | 187 | 2.518 | 15.251 | 18.259 | 1.00 | 0.75 | 1SG | 1531 |
| ATOM | 1531 | CA | PHE | 187 | 3.741 | 15.876 | 18.643 | 1.00 | 0.75 | 1SG | 1532 |
| ATOM | 1532 | CB | PHE | 187 | 4.969 | 14.969 | 18.396 | 1.00 | 0.75 | 1SG | 1533 |
| ATOM | 1533 | CG | PHE | 187 | 5.032 | 14.565 | 16.963 | 1.00 | 0.75 | 1SG | 1534 |
| ATOM | 1534 | CD1 | PHE | 187 | 4.237 | 13.544 | 16.494 | 1.00 | 0.75 | 1SG | 1535 |
| ATOM | 1535 | CD2 | PHE | 187 | 5.863 | 15.213 | 16.078 | 1.00 | 0.75 | 1SG | 1536 |
| ATOM | 1536 | CE1 | PHE | 187 | 4.281 | 13.166 | 15.172 | 1.00 | 0.75 | 1SG | 1537 |
| ATOM | 1537 | CE2 | PHE | 187 | 5.913 | 14.840 | 14.754 | 1.00 | 0.75 | 1SG | 1538 |
| ATOM | 1538 | CZ | PHE | 187 | 5.121 | 13.814 | 14.297 | 1.00 | 0.75 | 1SG | 1539 |
| ATOM | 1539 | C | PHE | 187 | 3.826 | 17.146 | 17.863 | 1.00 | 0.75 | 1SG | 1540 |
| ATOM | 1540 | O | PHE | 187 | 3.891 | 18.237 | 18.417 | 1.00 | 0.75 | 1SG | 1541 |
| ATOM | 1541 | N | ILE | 188 | 3.762 | 17.040 | 16.536 | 1.00 | 0.74 | 1SG | 1542 |
| ATOM | 1542 | CA | ILE | 188 | 3.682 | 18.189 | 15.700 | 1.00 | 0.74 | 1SG | 1543 |
| ATOM | 1543 | CB | ILE | 188 | 5.021 | 18.836 | 15.445 | 1.00 | 0.74 | 1SG | 1544 |
| ATOM | 1544 | CG2 | ILE | 188 | 5.958 | 17.839 | 14.739 | 1.00 | 0.74 | 1SG | 1545 |
| ATOM | 1545 | CG1 | ILE | 188 | 4.842 | 20.185 | 14.730 | 1.00 | 0.74 | 1SG | 1546 |
| ATOM | 1546 | CD1 | ILE | 188 | 6.100 | 21.055 | 14.749 | 1.00 | 0.74 | 1SG | 1547 |
| ATOM | 1547 | C | ILE | 188 | 3.126 | 17.614 | 14.453 | 1.00 | 0.74 | 1SG | 1548 |
| ATOM | 1548 | O | ILE | 188 | 3.608 | 17.845 | 13.344 | 1.00 | 0.74 | 1SG | 1549 |
| ATOM | 1549 | N | ALA | 189 | 2.040 | 16.843 | 14.626 | 1.00 | 0.59 | 1SG | 1550 |
| ATOM | 1550 | CA | ALA | 189 | 1.558 | 16.155 | 13.486 | 1.00 | 0.59 | 1SG | 1551 |
| ATOM | 1551 | CB | ALA | 189 | 0.934 | 14.801 | 13.796 | 1.00 | 0.59 | 1SG | 1552 |
| ATOM | 1552 | C | ALA | 189 | 0.523 | 16.974 | 12.828 | 1.00 | 0.59 | 1SG | 1553 |
| ATOM | 1553 | O | ALA | 189 | −0.654 | 16.835 | 13.134 | 1.00 | 0.59 | 1SG | 1554 |
| ATOM | 1554 | N | SER | 190 | 0.933 | 17.855 | 11.898 | 1.00 | 0.73 | 1SG | 1555 |
| ATOM | 1555 | CA | SER | 190 | −0.046 | 18.591 | 11.158 | 1.00 | 0.73 | 1SG | 1556 |
| ATOM | 1556 | CB | SER | 190 | 0.583 | 19.467 | 10.071 | 1.00 | 0.73 | 1SG | 1557 |
| ATOM | 1557 | OG | SER | 190 | 1.443 | 20.432 | 10.659 | 1.00 | 0.73 | 1SG | 1558 |
| ATOM | 1558 | C | SER | 190 | −0.827 | 17.516 | 10.496 | 1.00 | 0.73 | 1SG | 1559 |
| ATOM | 1559 | O | SER | 190 | −2.056 | 17.538 | 10.465 | 1.00 | 0.73 | 1SG | 1560 |
| ATOM | 1560 | N | LYS | 191 | −0.093 | 16.524 | 9.966 | 1.00 | 0.94 | 1SG | 1561 |
| ATOM | 1561 | CA | LYS | 191 | −0.711 | 15.338 | 9.478 | 1.00 | 0.94 | 1SG | 1562 |
| ATOM | 1562 | CB | LYS | 191 | −0.049 | 14.774 | 8.210 | 1.00 | 0.94 | 1SG | 1563 |
| ATOM | 1563 | CG | LYS | 191 | −0.198 | 15.707 | 7.003 | 1.00 | 0.94 | 1SG | 1564 |
| ATOM | 1564 | CD | LYS | 191 | 0.621 | 15.299 | 5.777 | 1.00 | 0.94 | 1SG | 1565 |
| ATOM | 1565 | CE | LYS | 191 | 0.417 | 16.232 | 4.583 | 1.00 | 0.94 | 1SG | 1566 |
| ATOM | 1566 | NZ | LYS | 191 | −1.028 | 16.405 | 4.321 | 1.00 | 0.94 | 1SG | 1567 |
| ATOM | 1567 | C | LYS | 191 | −0.500 | 14.387 | 10.607 | 1.00 | 0.94 | 1SG | 1568 |
| ATOM | 1568 | O | LYS | 191 | 0.560 | 13.777 | 10.734 | 1.00 | 0.94 | 1SG | 1569 |
| ATOM | 1569 | N | LEU | 192 | −1.521 | 14.271 | 11.476 | 1.00 | 0.99 | 1SG | 1570 |
| ATOM | 1570 | CA | LEU | 192 | −1.446 | 13.491 | 12.678 | 1.00 | 0.99 | 1SG | 1571 |
| ATOM | 1571 | CB | LEU | 192 | −2.720 | 13.542 | 13.541 | 1.00 | 0.99 | 1SG | 1572 |
| ATOM | 1572 | CG | LEU | 192 | −3.268 | 14.926 | 13.919 | 1.00 | 0.99 | 1SG | 1573 |
| ATOM | 1573 | CD2 | LEU | 192 | −3.635 | 15.734 | 12.667 | 1.00 | 0.99 | 1SG | 1574 |
| ATOM | 1574 | CD1 | LEU | 192 | −2.341 | 15.665 | 14.880 | 1.00 | 0.99 | 1SG | 1575 |
| ATOM | 1575 | C | LEU | 192 | −1.407 | 12.053 | 12.319 | 1.00 | 0.99 | 1SG | 1576 |
| ATOM | 1576 | O | LEU | 192 | −1.615 | 11.663 | 11.173 | 1.00 | 0.99 | 1SG | 1577 |
| ATOM | 1577 | N | VAL | 193 | −1.103 | 11.237 | 13.335 | 1.00 | 0.90 | 1SG | 1578 |
| ATOM | 1578 | CA | VAL | 193 | −1.271 | 9.835 | 13.239 | 1.00 | 0.90 | 1SG | 1579 |
| ATOM | 1579 | CB | VAL | 193 | −0.016 | 9.081 | 13.547 | 1.00 | 0.90 | 1SG | 1580 |
| ATOM | 1580 | CG1 | VAL | 193 | 1.037 | 9.453 | 12.497 | 1.00 | 0.90 | 1SG | 1581 |
| ATOM | 1531 | CG2 | VAL | 193 | 0.435 | 9.384 | 14.975 | 1.00 | 0.90 | 1SG | 1582 |
| ATOM | 1582 | C | VAL | 193 | −2.274 | 9.568 | 14.313 | 1.00 | 0.90 | 1SG | 1583 |
| ATOM | 1583 | O | VAL | 193 | −2.183 | 8.583 | 15.043 | 1.00 | 0.90 | 1SG | 1584 |
| ATOM | 1584 | N | ARG | 194 | −3.288 | 10.454 | 14.407 | 1.00 | 0.67 | 1SG | 1585 |
| ATOM | 1585 | CA | ARG | 194 | −4.236 | 10.338 | 15.472 | 1.00 | 0.67 | 1SG | 1586 |
| ATOM | 1586 | CB | ARG | 194 | −4.561 | 11.644 | 16.221 | 1.00 | 0.67 | 1SG | 1587 |
| ATOM | 1587 | CG | ARG | 194 | −3.529 | 12.197 | 17.202 | 1.00 | 0.67 | 1SG | 1588 |
| ATOM | 1588 | CD | ARG | 194 | −3.990 | 13.536 | 17.778 | 1.00 | 0.67 | 1SG | 1589 |
| ATOM | 1589 | NE | ARG | 194 | −4.222 | 14.411 | 16.593 | 1.00 | 0.67 | 1SG | 1590 |
| ATOM | 1590 | CZ | ARG | 194 | −5.225 | 15.338 | 16.524 | 1.00 | 0.67 | 1SG | 1591 |
| ATOM | 1591 | NH1 | ARG | 194 | −6.075 | 15.484 | 17.582 | 1.00 | 0.67 | 1SG | 1592 |
| ATOM | 1592 | NH2 | ARG | 194 | −5.346 | 16.148 | 15.424 | 1.00 | 0.67 | 1SG | 1593 |
| ATOM | 1593 | C | ARG | 194 | −5.574 | 9.954 | 14.960 | 1.00 | 0.67 | 1SG | 1594 |
| ATOM | 1594 | O | ARG | 194 | −5.871 | 10.004 | 13.769 | 1.00 | 0.67 | 1SG | 1595 |
| ATOM | 1595 | N | VAL | 195 | −6.398 | 9.543 | 15.941 | 1.00 | 0.46 | 1SG | 1596 |
| ATOM | 1596 | CA | VAL | 195 | −7.790 | 9.239 | 15.828 | 1.00 | 0.46 | 1SG | 1597 |
| ATOM | 1597 | CB | VAL | 195 | −8.176 | 8.098 | 16.722 | 1.00 | 0.46 | 1SG | 1598 |
| ATOM | 1598 | CG1 | VAL | 195 | −9.703 | 8.016 | 16.833 | 1.00 | 0.46 | 1SG | 1599 |
| ATOM | 1599 | CG2 | VAL | 195 | −7.531 | 6.814 | 16.159 | 1.00 | 0.46 | 1SG | 1600 |
| ATOM | 1600 | C | VAL | 195 | −8.473 | 10.463 | 16.339 | 1.00 | 0.46 | 1SG | 1601 |
| ATOM | 1601 | O | VAL | 195 | −8.490 | 10.707 | 17.548 | 1.00 | 0.46 | 1SG | 1602 |
| ATOM | 1602 | N | VAL | 196 | −9.045 | 11.280 | 15.439 | 1.00 | 0.43 | 1SG | 1603 |
| ATOM | 1603 | CA | VAL | 196 | −9.676 | 12.510 | 15.842 | 1.00 | 0.43 | 1SG | 1604 |
| ATOM | 1604 | CB | VAL | 196 | −8.979 | 13.741 | 15.309 | 1.00 | 0.43 | 1SG | 1605 |
| ATOM | 1605 | CG1 | VAL | 196 | −9.687 | 15.020 | 15.779 | 1.00 | 0.43 | 1SG | 1606 |
| ATOM | 1606 | CG2 | VAL | 196 | −7.502 | 13.661 | 15.701 | 1.00 | 0.43 | 1SG | 1607 |
| ATOM | 1607 | C | VAL | 196 | −11.052 | 12.567 | 15.260 | 1.00 | 0.43 | 1SG | 1608 |
| ATOM | 1608 | O | VAL | 196 | −11.362 | 11.889 | 14.284 | 1.00 | 0.43 | 1SG | 1609 |
| ATOM | 1609 | N | TYR | 197 | −11.941 | 13.344 | 15.907 | 1.00 | 0.38 | 1SG | 1610 |
| ATOM | 1610 | CA | TYR | 197 | −13.191 | 13.644 | 15.271 | 1.00 | 0.38 | 1SG | 1611 |
| ATOM | 1611 | CB | TYR | 197 | −14.196 | 14.457 | 16.112 | 1.00 | 0.38 | 1SG | 1612 |
| ATOM | 1612 | CG | TYR | 197 | −15.074 | 13.623 | 16.975 | 1.00 | 0.38 | 1SG | 1613 |
| ATOM | 1613 | CD1 | TYR | 197 | −16.199 | 13.071 | 16.413 | 1.00 | 0.38 | 1SG | 1614 |
| ATOM | 1614 | CD2 | TYR | 197 | −14.826 | 13.430 | 18.315 | 1.00 | 0.38 | 1SG | 1615 |
| ATOM | 1615 | CE1 | TYR | 197 | −17.059 | 12.311 | 17.165 | 1.00 | 0.38 | 1SG | 1616 |
| ATOM | 1616 | CE2 | TYR | 197 | −15.688 | 12.667 | 19.071 | 1.00 | 0.38 | 1SG | 1617 |
| ATOM | 1617 | CZ | TYR | 197 | −16.803 | 12.108 | 18.495 | 1.00 | 0.38 | 1SG | 1618 |
| ATOM | 1618 | OH | TYR | 197 | −17.692 | 11.332 | 19.265 | 1.00 | 0.38 | 1SG | 1619 |
| ATOM | 1619 | C | TYR | 197 | −12.794 | 14.606 | 14.208 | 1.00 | 0.38 | 1SG | 1620 |
| ATOM | 1620 | O | TYR | 197 | −12.574 | 15.782 | 14.496 | 1.00 | 0.38 | 1SG | 1621 |
| ATOM | 1621 | N | ALA | 198 | −12.695 | 14.141 | 12.950 | 1.00 | 0.50 | 1SG | 1622 |
| ATOM | 1622 | CA | ALA | 198 | −12.219 | 15.028 | 11.933 | 1.00 | 0.50 | 1SG | 1623 |
| ATOM | 1623 | CB | ALA | 198 | −12.084 | 14.361 | 10.553 | 1.00 | 0.50 | 1SG | 1624 |
| ATOM | 1624 | C | ALA | 198 | −13.163 | 16.170 | 11.784 | 1.00 | 0.50 | 1SG | 1625 |
| ATOM | 1625 | O | ALA | 198 | −12.750 | 17.330 | 11.772 | 1.00 | 0.50 | 1SG | 1626 |
| ATOM | 1626 | N | SER | 199 | −14.470 | 15.892 | 11.683 | 1.00 | 0.83 | 1SG | 1627 |
| ATOM | 1627 | CA | SER | 199 | −15.329 | 17.012 | 11.480 | 1.00 | 0.83 | 1SG | 1628 |
| ATOM | 1628 | CB | SER | 199 | −15.231 | 17.641 | 10.088 | 1.00 | 0.83 | 1SG | 1629 |
| ATOM | 1629 | OG | SER | 199 | −16.384 | 18.435 | 9.887 | 1.00 | 0.83 | 1SG | 1630 |
| ATOM | 1630 | C | SER | 199 | −16.744 | 16.609 | 11.604 | 1.00 | 0.83 | 1SG | 1631 |
| ATOM | 1631 | O | SER | 199 | −17.308 | 15.982 | 10.714 | 1.00 | 0.83 | 1SG | 1632 |
| ATOM | 1632 | N | TRP | 200 | −17.346 | 16.975 | 12.737 | 1.00 | 1.01 | 1SG | 1633 |
| ATOM | 1633 | CA | TRP | 200 | −18.732 | 16.781 | 12.941 | 1.00 | 1.01 | 1SG | 1634 |
| ATOM | 1634 | CB | TRP | 200 | −18.959 | 15.308 | 13.377 | 1.00 | 1.01 | 1SG | 1635 |
| ATOM | 1635 | CG | TRP | 200 | −19.966 | 14.776 | 14.348 | 1.00 | 1.01 | 1SG | 1636 |
| ATOM | 1636 | CD2 | TRP | 200 | −21.136 | 15.453 | 14.832 | 1.00 | 1.01 | 1SG | 1637 |
| ATOM | 1637 | CD1 | TRP | 200 | −19.829 | 13.646 | 15.106 | 1.00 | 1.01 | 1SG | 1638 |
| ATOM | 1638 | NE1 | TRP | 200 | −20.874 | 13.533 | 15.978 | 1.00 | 1.01 | 1SG | 1639 |
| ATOM | 1639 | CE2 | TRP | 200 | −21.677 | 14.652 | 15.844 | 1.00 | 1.01 | 1SG | 1640 |
| ATOM | 1640 | CE3 | TRP | 200 | −21.677 | 16.665 | 14.499 | 1.00 | 1.01 | 1SG | 1641 |
| ATOM | 1641 | CZ2 | TRP | 200 | −22.779 | 15.053 | 16.545 | 1.00 | 1.01 | 1SG | 1642 |
| ATOM | 1642 | CZ3 | TRP | 200 | −22.822 | 17.042 | 15.177 | 1.00 | 1.01 | 1SG | 1643 |
| ATOM | 1643 | CH2 | TRP | 200 | −23.355 | 16.250 | 16.178 | 1.00 | 1.01 | 1SG | 1644 |
| ATOM | 1644 | C | TRP | 200 | −19.161 | 18.044 | 13.627 | 1.00 | 1.01 | 1SG | 1645 |
| ATOM | 1645 | O | TRP | 200 | −19.238 | 18.184 | 14.845 | 1.00 | 1.01 | 1SG | 1646 |
| ATOM | 1646 | N | SER | 201 | −19.406 | 19.004 | 12.685 | 1.00 | 1.14 | 1SG | 1647 |
| ATOM | 1647 | CA | SER | 201 | −19.462 | 20.446 | 12.636 | 1.00 | 1.14 | 1SG | 1648 |
| ATOM | 1648 | CB | SER | 201 | −20.158 | 20.984 | 11.367 | 1.00 | 1.14 | 1SG | 1649 |
| ATOM | 1649 | OG | SER | 201 | −19.307 | 20.876 | 10.232 | 1.00 | 1.14 | 1SG | 1650 |
| ATOM | 1650 | C | SER | 201 | −20.002 | 21.263 | 13.763 | 1.00 | 1.14 | 1SG | 1651 |
| ATOM | 1651 | O | SER | 201 | −19.221 | 21.719 | 14.594 | 1.00 | 1.14 | 1SG | 1652 |
| ATOM | 1652 | N | ARG | 202 | −21.322 | 21.478 | 13.867 | 1.00 | 1.13 | 1SG | 1653 |
| ATOM | 1653 | CA | ARG | 202 | −21.581 | 22.608 | 14.710 | 1.00 | 1.13 | 1SG | 1654 |
| ATOM | 1654 | CB | ARG | 202 | −22.393 | 23.711 | 13.995 | 1.00 | 1.13 | 1SG | 1655 |
| ATOM | 1655 | CG | ARG | 202 | −23.767 | 23.264 | 13.498 | 1.00 | 1.13 | 1SG | 1656 |
| ATOM | 1656 | CD | ARG | 202 | −24.118 | 23.789 | 12.101 | 1.00 | 1.13 | 1SG | 1657 |
| ATOM | 1657 | NE | ARG | 202 | −24.281 | 25.272 | 12.147 | 1.00 | 1.13 | 1SG | 1658 |
| ATOM | 1658 | CZ | ARG | 202 | −23.950 | 26.021 | 11.053 | 1.00 | 1.13 | 1SG | 1659 |
| ATOM | 1659 | NH1 | ARG | 202 | −23.350 | 25.439 | 9.974 | 1.00 | 1.13 | 1SG | 1660 |
| ATOM | 1660 | NH2 | ARG | 202 | −24.221 | 27.357 | 11.032 | 1.00 | 1.13 | 1SG | 1661 |
| ATOM | 1661 | C | ARG | 202 | −22.216 | 22.300 | 16.017 | 1.00 | 1.13 | 1SG | 1662 |
| ATOM | 1662 | O | ARG | 202 | −23.440 | 22.240 | 16.124 | 1.00 | 1.13 | 1SG | 1663 |
| ATOM | 1663 | N | VAL | 203 | −21.352 | 22.144 | 17.046 | 1.00 | 0.79 | 1SG | 1664 |
| ATOM | 1664 | CA | VAL | 203 | −21.708 | 22.002 | 18.430 | 1.00 | 0.79 | 1SG | 1665 |
| ATOM | 1665 | CB | VAL | 203 | −22.233 | 20.643 | 18.850 | 1.00 | 0.79 | 1SG | 1666 |
| ATOM | 1666 | CG1 | VAL | 203 | −22.833 | 20.745 | 20.272 | 1.00 | 0.79 | 1SG | 1667 |
| ATOM | 1667 | CG2 | VAL | 203 | −23.254 | 20.120 | 17.831 | 1.00 | 0.79 | 1SG | 1668 |
| ATOM | 1668 | C | VAL | 203 | −20.388 | 22.221 | 19.126 | 1.00 | 0.79 | 1SG | 1669 |
| ATOM | 1669 | O | VAL | 203 | −19.572 | 23.028 | 18.683 | 1.00 | 0.79 | 1SG | 1670 |
| ATOM | 1670 | N | GLN | 204 | −20.151 | 21.499 | 20.235 | 1.00 | 0.58 | 1SG | 1671 |
| ATOM | 1671 | CA | GLN | 204 | −18.967 | 21.543 | 21.035 | 1.00 | 0.58 | 1SG | 1672 |
| ATOM | 1672 | CB | GLN | 204 | −19.165 | 20.794 | 22.366 | 1.00 | 0.58 | 1SG | 1673 |
| ATOM | 1673 | CG | GLN | 204 | −19.552 | 19.322 | 22.192 | 1.00 | 0.58 | 1SG | 1674 |
| ATOM | 1674 | CD | GLN | 204 | −20.139 | 18.841 | 23.514 | 1.00 | 0.58 | 1SG | 1675 |
| ATOM | 1675 | OE1 | GLN | 204 | −20.419 | 19.640 | 24.407 | 1.00 | 0.58 | 1SG | 1676 |
| ATOM | 1676 | NE2 | GLN | 204 | −20.350 | 17.504 | 23.644 | 1.00 | 0.58 | 1SG | 1677 |
| ATOM | 1677 | C | GLN | 204 | −17.834 | 20.930 | 20.273 | 1.00 | 0.58 | 1SG | 1678 |
| ATOM | 1678 | O | GLN | 204 | −16.673 | 21.256 | 20.523 | 1.00 | 0.58 | 1SG | 1679 |
| ATOM | 1679 | N | ALA | 205 | −18.133 | 20.026 | 19.318 | 1.00 | 0.55 | 1SG | 1680 |
| ATOM | 1680 | CA | ALA | 205 | −17.075 | 19.385 | 18.597 | 1.00 | 0.55 | 1SG | 1681 |
| ATOM | 1681 | CB | ALA | 205 | −17.590 | 18.425 | 17.504 | 1.00 | 0.55 | 1SG | 1682 |
| ATOM | 1682 | C | ALA | 205 | −16.273 | 20.447 | 17.927 | 1.00 | 0.55 | 1SG | 1683 |
| ATOM | 1683 | O | ALA | 205 | −15.079 | 20.556 | 18.191 | 1.00 | 0.55 | 1SG | 1684 |
| ATOM | 1684 | N | ASP | 206 | −16.940 | 21.298 | 17.118 | 1.00 | 0.56 | 1SG | 1685 |
| ATOM | 1685 | CA | ASP | 206 | −16.317 | 22.363 | 16.375 | 1.00 | 0.56 | 1SG | 1686 |
| ATOM | 1686 | CB | ASP | 206 | −15.921 | 23.567 | 17.230 | 1.00 | 0.56 | 1SG | 1687 |
| ATOM | 1687 | CG | ASP | 206 | −17.179 | 24.353 | 17.575 | 1.00 | 0.56 | 1SG | 1688 |
| ATOM | 1688 | OD1 | ASP | 206 | −18.033 | 24.547 | 16.670 | 1.00 | 0.56 | 1SG | 1689 |
| ATOM | 1689 | OD2 | ASP | 206 | −17.302 | 24.769 | 18.755 | 1.00 | 0.56 | 1SG | 1690 |
| ATOM | 1690 | C | ASP | 206 | −15.120 | 21.861 | 15.631 | 1.00 | 0.56 | 1SG | 1691 |
| ATOM | 1691 | O | ASP | 206 | −14.040 | 21.645 | 16.178 | 1.00 | 0.56 | 1SG | 1692 |
| ATOM | 1692 | N | LEU | 207 | −15.298 | 21.717 | 14.309 | 1.00 | 0.59 | 1SG | 1693 |
| ATOM | 1693 | CA | LEU | 207 | −14.327 | 21.090 | 13.467 | 1.00 | 0.59 | 1SG | 1694 |
| ATOM | 1694 | CB | LEU | 207 | −14.717 | 21.251 | 11.994 | 1.00 | 0.59 | 1SG | 1695 |
| ATOM | 1695 | CG | LEU | 207 | −16.204 | 20.999 | 11.738 | 1.00 | 0.59 | 1SG | 1696 |
| ATOM | 1696 | CD2 | LEU | 207 | −16.642 | 19.671 | 12.356 | 1.00 | 0.59 | 1SG | 1697 |
| ATOM | 1697 | CD1 | LEU | 207 | −16.544 | 21.179 | 10.253 | 1.00 | 0.59 | 1SG | 1698 |
| ATOM | 1698 | C | LEU | 207 | −13.010 | 21.790 | 13.562 | 1.00 | 0.59 | 1SG | 1699 |
| ATOM | 1699 | O | LEU | 207 | −12.044 | 21.269 | 14.117 | 1.00 | 0.59 | 1SG | 1700 |
| ATOM | 1700 | N | ASN | 208 | −12.955 | 23.028 | 13.042 | 1.00 | 0.53 | 1SG | 1701 |
| ATOM | 1701 | CA | ASN | 208 | −11.701 | 23.718 | 13.010 | 1.00 | 0.53 | 1SG | 1702 |
| ATOM | 1702 | CB | ASN | 208 | −11.761 | 25.012 | 12.184 | 1.00 | 0.53 | 1SG | 1703 |
| ATOM | 1703 | CG | ASN | 208 | −10.344 | 25.555 | 12.050 | 1.00 | 0.53 | 1SG | 1704 |
| ATOM | 1704 | OD1 | ASN | 208 | −9.672 | 25.818 | 13.046 | 1.00 | 0.53 | 1SG | 1705 |
| ATOM | 1705 | ND2 | ASN | 208 | −9.871 | 25.728 | 10.786 | 1.00 | 0.53 | 1SG | 1706 |
| ATOM | 1706 | C | ASN | 208 | −11.318 | 24.083 | 14.395 | 1.00 | 0.53 | 1SG | 1707 |
| ATOM | 1707 | O | ASN | 208 | −10.245 | 23.739 | 14.887 | 1.00 | 0.53 | 1SG | 1708 |
| ATOM | 1708 | N | CYS | 209 | −12.235 | 24.785 | 15.075 | 1.00 | 0.51 | 1SG | 1709 |
| ATOM | 1709 | CA | CYS | 209 | −12.000 | 25.226 | 16.410 | 1.00 | 0.51 | 1SG | 1710 |
| ATOM | 1710 | CB | CYS | 209 | −11.260 | 26.566 | 16.490 | 1.00 | 0.51 | 1SG | 1711 |
| ATOM | 1711 | SG | CYS | 209 | −10.988 | 27.108 | 18.201 | 1.00 | 0.51 | 1SG | 1712 |
| ATOM | 1712 | C | CYS | 209 | −13.342 | 25.437 | 16.991 | 1.00 | 0.51 | 1SG | 1713 |
| ATOM | 1713 | O | CYS | 209 | −14.322 | 25.571 | 16.262 | 1.00 | 0.51 | 1SG | 1714 |
| ATOM | 1714 | N | MET | 210 | −13.425 | 25.467 | 18.328 | 1.00 | 0.63 | 1SG | 1715 |
| ATOM | 1715 | CA | MET | 210 | −14.713 | 25.650 | 18.908 | 1.00 | 0.63 | 1SG | 1716 |
| ATOM | 1716 | CB | MET | 210 | −14.692 | 25.639 | 20.450 | 1.00 | 0.63 | 1SG | 1717 |
| ATOM | 1717 | CG | MET | 210 | −13.859 | 26.764 | 21.070 | 1.00 | 0.63 | 1SG | 1718 |
| ATOM | 1718 | SD | MET | 210 | −13.818 | 26.751 | 22.888 | 1.00 | 0.63 | 1SG | 1719 |
| ATOM | 1719 | CE | MET | 210 | −12.955 | 28.340 | 23.059 | 1.00 | 0.63 | 1SG | 1720 |
| ATOM | 1720 | C | MET | 210 | −15.240 | 26.968 | 18.449 | 1.00 | 0.63 | 1SG | 1721 |
| ATOM | 1721 | O | MET | 210 | −16.378 | 27.075 | 17.991 | 1.00 | 0.63 | 1SG | 1722 |
| ATOM | 1722 | N | GLU | 211 | −14.402 | 28.014 | 18.534 | 1.00 | 0.70 | 1SG | 1723 |
| ATOM | 1723 | CA | GLU | 211 | −14.883 | 29.305 | 18.159 | 1.00 | 0.70 | 1SG | 1724 |
| ATOM | 1724 | CB | GLU | 211 | −13.940 | 30.460 | 18.542 | 1.00 | 0.70 | 1SG | 1725 |
| ATOM | 1725 | CG | GLU | 211 | −13.909 | 30.708 | 20.053 | 1.00 | 0.70 | 1SG | 1726 |
| ATOM | 1726 | CD | GLU | 211 | −13.219 | 32.038 | 20.308 | 1.00 | 0.70 | 1SG | 1727 |
| ATOM | 1727 | OE1 | GLU | 211 | −12.342 | 32.414 | 19.486 | 1.00 | 0.70 | 1SG | 1728 |
| ATOM | 1728 | OE2 | GLU | 211 | −13.563 | 32.700 | 21.324 | 1.00 | 0.70 | 1SG | 1729 |
| ATOM | 1729 | C | GLU | 211 | −15.161 | 29.363 | 16.696 | 1.00 | 0.70 | 1SG | 1730 |
| ATOM | 1730 | O | GLU | 211 | −16.244 | 29.783 | 16.295 | 1.00 | 0.70 | 1SG | 1731 |
| ATOM | 1731 | N | ASP | 212 | −14.221 | 28.934 | 15.832 | 1.00 | 0.87 | 1SG | 1732 |
| ATOM | 1732 | CA | ASP | 212 | −14.584 | 29.073 | 14.453 | 1.00 | 0.87 | 1SG | 1733 |
| ATOM | 1733 | CB | ASP | 212 | −14.823 | 30.547 | 14.064 | 1.00 | 0.87 | 1SG | 1734 |
| ATOM | 1734 | CG | ASP | 212 | −15.625 | 30.641 | 12.768 | 1.00 | 0.87 | 1SG | 1735 |
| ATOM | 1735 | OD1 | ASP | 212 | −16.123 | 29.589 | 12.287 | 1.00 | 0.87 | 1SG | 1736 |
| ATOM | 1736 | OD2 | ASP | 212 | −15.749 | 31.780 | 12.245 | 1.00 | 0.87 | 1SG | 1737 |
| ATOM | 1737 | C | ASP | 212 | −13.474 | 28.555 | 13.599 | 1.00 | 0.87 | 1SG | 1738 |
| ATOM | 1738 | O | ASP | 212 | −12.383 | 28.247 | 14.079 | 1.00 | 0.87 | 1SG | 1739 |
| ATOM | 1739 | N | LEU | 213 | −13.773 | 28.429 | 12.291 | 1.00 | 0.99 | 1SG | 1740 |
| ATOM | 1740 | CA | LEU | 213 | −12.846 | 28.021 | 11.280 | 1.00 | 0.99 | 1SG | 1741 |
| ATOM | 1741 | CB | LEU | 213 | −13.565 | 27.549 | 9.999 | 1.00 | 0.99 | 1SG | 1742 |
| ATOM | 1742 | CG | LEU | 213 | −14.673 | 28.500 | 9.492 | 1.00 | 0.99 | 1SG | 1743 |
| ATOM | 1743 | CD2 | LEU | 213 | −15.569 | 27.787 | 8.470 | 1.00 | 0.99 | 1SG | 1744 |
| ATOM | 1744 | CD1 | LEU | 213 | −14.112 | 29.829 | 8.957 | 1.00 | 0.99 | 1SG | 1745 |
| ATOM | 1745 | C | LEU | 213 | −12.044 | 29.234 | 10.958 | 1.00 | 0.99 | 1SG | 1746 |
| ATOM | 1746 | O | LEU | 213 | −12.474 | 30.346 | 11.259 | 1.00 | 0.99 | 1SG | 1747 |
| ATOM | 1747 | N | LEU | 214 | −10.847 | 29.075 | 10.356 | 1.00 | 0.71 | 1SG | 1748 |
| ATOM | 1748 | CA | LEU | 214 | −10.133 | 30.283 | 10.049 | 1.00 | 0.71 | 1SG | 1749 |
| ATOM | 1749 | CB | LEU | 214 | −9.010 | 30.589 | 11.067 | 1.00 | 0.71 | 1SG | 1750 |
| ATOM | 1750 | CG | LEU | 214 | −8.506 | 32.050 | 11.080 | 1.00 | 0.71 | 1SG | 1751 |
| ATOM | 1751 | CD2 | LEU | 214 | −7.999 | 32.512 | 9.705 | 1.00 | 0.71 | 1SG | 1752 |
| ATOM | 1752 | CD1 | LEU | 214 | −7.456 | 32.260 | 12.180 | 1.00 | 0.71 | 1SG | 1753 |
| ATOM | 1753 | C | LEU | 214 | −9.592 | 30.156 | 8.651 | 1.00 | 0.71 | 1SG | 1754 |
| ATOM | 1754 | O | LEU | 214 | −9.092 | 29.104 | 8.256 | 1.00 | 0.71 | 1SG | 1755 |
| ATOM | 1755 | N | GLN | 215 | −9.728 | 31.248 | 7.867 | 1.00 | 0.45 | 1SG | 1756 |
| ATOM | 1756 | CA | GLN | 215 | −9.375 | 31.368 | 6.474 | 1.00 | 0.45 | 1SG | 1757 |
| ATOM | 1757 | CB | GLN | 215 | −9.703 | 32.780 | 5.942 | 1.00 | 0.45 | 1SG | 1758 |
| ATOM | 1758 | CG | GLN | 215 | −11.186 | 33.160 | 6.035 | 1.00 | 0.45 | 1SG | 1759 |
| ATOM | 1759 | CD | GLN | 215 | −11.321 | 34.639 | 5.685 | 1.00 | 0.45 | 1SG | 1760 |
| ATOM | 1760 | OE1 | GLN | 215 | −10.341 | 35.382 | 5.685 | 1.00 | 0.45 | 1SG | 1761 |
| ATOM | 1761 | NE2 | GLN | 215 | −12.571 | 35.086 | 5.388 | 1.00 | 0.45 | 1SG | 1762 |
| ATOM | 1762 | C | GLN | 215 | −7.902 | 31.194 | 6.273 | 1.00 | 0.45 | 1SG | 1763 |
| ATOM | 1763 | O | GLN | 215 | −7.465 | 30.449 | 5.395 | 1.00 | 0.45 | 1SG | 1764 |
| ATOM | 1764 | N | SER | 216 | −7.096 | 31.872 | 7.104 | 1.00 | 0.52 | 1SG | 1765 |
| ATOM | 1765 | CA | SER | 216 | −5.678 | 31.874 | 6.914 | 1.00 | 0.52 | 1SG | 1766 |
| ATOM | 1766 | CB | SER | 216 | −4.960 | 32.945 | 7.753 | 1.00 | 0.52 | 1SG | 1767 |
| ATOM | 1767 | OG | SER | 216 | −5.120 | 32.670 | 9.137 | 1.00 | 0.52 | 1SG | 1768 |
| ATOM | 1768 | C | SER | 216 | −5.107 | 30.561 | 7.305 | 1.00 | 0.52 | 1SG | 1769 |
| ATOM | 1769 | O | SER | 216 | −5.813 | 29.599 | 7.597 | 1.00 | 0.52 | 1SG | 1770 |
| ATOM | 1770 | N | SER | 217 | −3.763 | 30.510 | 7.287 | 1.00 | 0.66 | 1SG | 1771 |
| ATOM | 1771 | CA | SER | 217 | −3.055 | 29.329 | 7.662 | 1.00 | 0.66 | 1SG | 1772 |
| ATOM | 1772 | CB | SER | 217 | −1.796 | 29.064 | 6.817 | 1.00 | 0.66 | 1SG | 1773 |
| ATOM | 1773 | OG | SER | 217 | −2.157 | 28.814 | 5.468 | 1.00 | 0.66 | 1SG | 1774 |
| ATOM | 1774 | C | SER | 217 | −2.585 | 29.534 | 9.057 | 1.00 | 0.66 | 1SG | 1775 |
| ATOM | 1775 | O | SER | 217 | −1.424 | 29.869 | 9.289 | 1.00 | 0.66 | 1SG | 1776 |
| ATOM | 1776 | N | VAL | 218 | −3.491 | 29.369 | 10.031 | 1.00 | 0.69 | 1SG | 1777 |
| ATOM | 1777 | CA | VAL | 218 | −3.058 | 29.447 | 11.387 | 1.00 | 0.69 | 1SG | 1778 |
| ATOM | 1778 | CB | VAL | 218 | −3.789 | 30.473 | 12.209 | 1.00 | 0.69 | 1SG | 1779 |
| ATOM | 1779 | CG1 | VAL | 218 | −5.303 | 30.239 | 12.097 | 1.00 | 0.69 | 1SG | 1780 |
| ATOM | 1780 | CG2 | VAL | 218 | −3.263 | 30.407 | 13.650 | 1.00 | 0.69 | 1SG | 1781 |
| ATOM | 1781 | C | VAL | 218 | −3.264 | 28.085 | 11.959 | 1.00 | 0.69 | 1SG | 1782 |
| ATOM | 1782 | O | VAL | 218 | −4.351 | 27.694 | 12.380 | 1.00 | 0.69 | 1SG | 1783 |
| ATOM | 1783 | N | PRO | 219 | −2.183 | 27.363 | 11.969 | 1.00 | 0.66 | 1SG | 1784 |
| ATOM | 1784 | CA | PRO | 219 | −2.181 | 26.002 | 12.428 | 1.00 | 0.66 | 1SG | 1785 |
| ATOM | 1785 | CD | PRO | 219 | −0.878 | 27.986 | 12.113 | 1.00 | 0.66 | 1SG | 1786 |
| ATOM | 1786 | CB | PRO | 219 | −0.712 | 25.590 | 12.453 | 1.00 | 0.66 | 1SG | 1787 |
| ATOM | 1787 | CG | PRO | 219 | 0.006 | 26.914 | 12.774 | 1.00 | 0.66 | 1SG | 1788 |
| ATOM | 1788 | C | PRO | 219 | −2.748 | 25.999 | 13.813 | 1.00 | 0.66 | 1SG | 1789 |
| ATOM | 1789 | O | PRO | 219 | −2.768 | 27.054 | 14.444 | 1.00 | 0.66 | 1SG | 1790 |
| ATOM | 1790 | N | TRP | 220 | −3.241 | 24.845 | 14.302 | 1.00 | 0.84 | 1SG | 1791 |
| ATOM | 1791 | CA | TRP | 220 | −3.817 | 24.820 | 15.619 | 1.00 | 0.84 | 1SG | 1792 |
| ATOM | 1792 | CB | TRP | 220 | −5.329 | 24.514 | 15.600 | 1.00 | 0.84 | 1SG | 1793 |
| ATOM | 1793 | CG | TRP | 220 | −5.737 | 23.449 | 14.606 | 1.00 | 0.84 | 1SG | 1794 |
| ATOM | 1794 | CD2 | TRP | 220 | −5.623 | 22.030 | 14.800 | 1.00 | 0.84 | 1SG | 1795 |
| ATOM | 1795 | CD1 | TRP | 220 | −6.253 | 23.624 | 13.354 | 1.00 | 0.84 | 1SG | 1796 |
| ATOM | 1796 | NE1 | TRP | 220 | −6.467 | 22.407 | 12.756 | 1.00 | 0.84 | 1SG | 1797 |
| ATOM | 1797 | CE2 | TRP | 220 | −6.082 | 21.416 | 13.633 | 1.00 | 0.84 | 1SG | 1798 |
| ATOM | 1798 | CE3 | TRP | 220 | −5.168 | 21.299 | 15.858 | 1.00 | 0.84 | 1SG | 1799 |
| ATOM | 1799 | CZ2 | TRP | 220 | −6.092 | 20.056 | 13.509 | 1.00 | 0.84 | 1SG | 1800 |
| ATOM | 1800 | CZ3 | TRP | 220 | −5.186 | 19.928 | 15.731 | 1.00 | 0.84 | 1SG | 1801 |
| ATOM | 1801 | CH2 | TRP | 220 | −5.638 | 19.318 | 14.579 | 1.00 | 0.84 | 1SG | 1802 |
| ATOM | 1802 | C | TRP | 220 | −3.109 | 23.841 | 16.534 | 1.00 | 0.84 | 1SG | 1803 |
| ATOM | 1803 | O | TRP | 220 | −2.817 | 22.718 | 16.128 | 1.00 | 0.84 | 1SG | 1804 |
| ATOM | 1804 | N | LYS | 221 | −2.798 | 24.278 | 17.792 | 1.00 | 1.08 | 1SG | 1805 |
| ATOM | 1805 | CA | LYS | 221 | −2.175 | 23.489 | 18.839 | 1.00 | 1.08 | 1SG | 1806 |
| ATOM | 1806 | CB | LYS | 221 | −0.654 | 23.329 | 18.644 | 1.00 | 1.08 | 1SG | 1807 |
| ATOM | 1807 | CG | LYS | 221 | 0.134 | 24.639 | 18.701 | 1.00 | 1.08 | 1SG | 1808 |
| ATOM | 1808 | CD | LYS | 221 | 1.644 | 24.432 | 18.560 | 1.00 | 1.08 | 1SG | 1809 |
| ATOM | 1809 | CE | LYS | 221 | 2.203 | 23.309 | 19.436 | 1.00 | 1.08 | 1SG | 1810 |
| ATOM | 1810 | NZ | LYS | 221 | 3.653 | 23.151 | 19.184 | 1.00 | 1.08 | 1SG | 1811 |
| ATOM | 1811 | C | LYS | 221 | −2.434 | 24.206 | 20.134 | 1.00 | 1.08 | 1SG | 1812 |
| ATOM | 1812 | O | LYS | 221 | −1.514 | 24.506 | 20.896 | 1.00 | 1.08 | 1SG | 1813 |
| ATOM | 1813 | N | TYR | 222 | −3.727 | 24.461 | 20.427 | 1.00 | 0.97 | 1SG | 1814 |
| ATOM | 1814 | CA | TYR | 222 | −4.103 | 25.228 | 21.579 | 1.00 | 0.97 | 1SG | 1815 |
| ATOM | 1815 | CB | TYR | 222 | −5.206 | 26.256 | 21.284 | 1.00 | 0.97 | 1SG | 1816 |
| ATOM | 1816 | CG | TYR | 222 | −4.717 | 27.154 | 20.202 | 1.00 | 0.97 | 1SG | 1817 |
| ATOM | 1817 | CD1 | TYR | 222 | −3.882 | 28.213 | 20.476 | 1.00 | 0.97 | 1SG | 1818 |
| ATOM | 1818 | CD2 | TYR | 222 | −5.110 | 26.934 | 18.901 | 1.00 | 0.97 | 1SG | 1819 |
| ATOM | 1819 | CE1 | TYR | 222 | −3.442 | 29.034 | 19.463 | 1.00 | 0.97 | 1SG | 1820 |
| ATOM | 1820 | CE2 | TYR | 222 | −4.674 | 27.751 | 17.886 | 1.00 | 0.97 | 1SG | 1821 |
| ATOM | 1821 | CZ | TYR | 222 | −3.837 | 28.804 | 18.166 | 1.00 | 0.97 | 1SG | 1822 |
| ATOM | 1822 | OH | TYR | 222 | −3.386 | 29.645 | 17.126 | 1.00 | 0.97 | 1SG | 1823 |
| ATOM | 1823 | C | TYR | 222 | −4.664 | 24.308 | 22.615 | 1.00 | 0.97 | 1SG | 1824 |
| ATOM | 1824 | O | TYR | 222 | −4.715 | 23.092 | 22.436 | 1.00 | 0.97 | 1SG | 1825 |
| ATOM | 1825 | N | PHE | 223 | −5.099 | 24.913 | 23.740 | 1.00 | 0.66 | 1SG | 1826 |
| ATOM | 1826 | CA | PHE | 223 | −5.620 | 24.232 | 24.888 | 1.00 | 0.66 | 1SG | 1827 |
| ATOM | 1827 | CB | PHE | 223 | −5.833 | 25.161 | 26.100 | 1.00 | 0.66 | 1SG | 1828 |
| ATOM | 1828 | CG | PHE | 223 | −6.818 | 26.223 | 25.752 | 1.00 | 0.66 | 1SG | 1829 |
| ATOM | 1829 | CD1 | PHE | 223 | −8.167 | 25.967 | 25.814 | 1.00 | 0.66 | 1SG | 1830 |
| ATOM | 1830 | CD2 | PHE | 223 | −6.394 | 27.478 | 25.378 | 1.00 | 0.66 | 1SG | 1831 |
| ATOM | 1831 | CE1 | PHE | 223 | −9.080 | 26.946 | 25.501 | 1.00 | 0.66 | 1SG | 1832 |
| ATOM | 1832 | CE2 | PHE | 223 | −7.304 | 28.460 | 25.064 | 1.00 | 0.66 | 1SG | 1833 |
| ATOM | 1833 | CZ | PHE | 223 | −8.651 | 28.195 | 25.124 | 1.00 | 0.66 | 1SG | 1834 |
| ATOM | 1834 | C | PHE | 223 | −6.905 | 23.549 | 24.560 | 1.00 | 0.66 | 1SG | 1835 |
| ATOM | 1835 | O | PHE | 223 | −7.219 | 22.517 | 25.152 | 1.00 | 0.66 | 1SG | 1836 |
| ATOM | 1836 | N | LEU | 224 | −7.673 | 24.093 | 23.599 | 1.00 | 0.54 | 1SG | 1837 |
| ATOM | 1837 | CA | LEU | 224 | −8.952 | 23.530 | 23.284 | 1.00 | 0.54 | 1SG | 1838 |
| ATOM | 1838 | CB | LEU | 224 | −9.615 | 24.220 | 22.087 | 1.00 | 0.54 | 1SG | 1839 |
| ATOM | 1839 | CG | LEU | 224 | −10.078 | 25.652 | 22.397 | 1.00 | 0.54 | 1SG | 1840 |
| ATOM | 1840 | CD2 | LEU | 224 | −10.983 | 25.675 | 23.638 | 1.00 | 0.54 | 1SG | 1841 |
| ATOM | 1841 | CD1 | LEU | 224 | −10.737 | 26.308 | 21.174 | 1.00 | 0.54 | 1SG | 1842 |
| ATOM | 1842 | C | LEU | 224 | −8.781 | 22.083 | 22.946 | 1.00 | 0.54 | 1SG | 1843 |
| ATOM | 1843 | O | LEU | 224 | −9.490 | 21.232 | 23.479 | 1.00 | 0.54 | 1SG | 1844 |
| ATOM | 1844 | N | ASN | 225 | −7.822 | 21.746 | 22.068 | 1.00 | 0.68 | 1SG | 1845 |
| ATOM | 1845 | CA | ASN | 225 | −7.700 | 20.354 | 21.752 | 1.00 | 0.68 | 1SG | 1846 |
| ATOM | 1846 | CB | ASN | 225 | −7.462 | 20.079 | 20.252 | 1.00 | 0.68 | 1SG | 1847 |
| ATOM | 1847 | CG | ASN | 225 | −6.229 | 20.833 | 19.771 | 1.00 | 0.68 | 1SG | 1848 |
| ATOM | 1848 | OD1 | ASN | 225 | −5.797 | 21.800 | 20.394 | 1.00 | 0.68 | 1SG | 1849 |
| ATOM | 1849 | ND2 | ASN | 225 | −5.668 | 20.403 | 18.608 | 1.00 | 0.68 | 1SG | 1850 |
| ATOM | 1850 | C | ASN | 225 | −6.600 | 19.755 | 22.571 | 1.00 | 0.68 | 1SG | 1851 |
| ATOM | 1851 | O | ASN | 225 | −5.464 | 20.220 | 22.518 | 1.00 | 0.68 | 1SG | 1852 |
| ATOM | 1852 | N | THR | 226 | −6.945 | 18.714 | 23.375 | 1.00 | 0.87 | 1SG | 1853 |
| ATOM | 1853 | CA | THR | 226 | −6.012 | 18.008 | 24.215 | 1.00 | 0.87 | 1SG | 1854 |
| ATOM | 1854 | CB | THR | 226 | −6.250 | 18.213 | 25.686 | 1.00 | 0.87 | 1SG | 1855 |
| ATOM | 1855 | OG1 | THR | 226 | −7.535 | 17.729 | 26.045 | 1.00 | 0.87 | 1SG | 1856 |
| ATOM | 1856 | CG2 | THR | 226 | −6.135 | 19.714 | 26.008 | 1.00 | 0.87 | 1SG | 1857 |
| ATOM | 1857 | C | THR | 226 | −6.165 | 16.547 | 23.919 | 1.00 | 0.87 | 1SG | 1858 |
| ATOM | 1858 | O | THR | 226 | −7.278 | 16.049 | 23.762 | 1.00 | 0.87 | 1SG | 1859 |
| ATOM | 1859 | N | CYS | 227 | −5.039 | 15.813 | 23.835 | 1.00 | 0.78 | 1SG | 1860 |
| ATOM | 1860 | CA | CYS | 227 | −5.121 | 14.427 | 23.486 | 1.00 | 0.78 | 1SG | 1861 |
| ATOM | 1861 | CB | CYS | 227 | −4.011 | 13.977 | 22.532 | 1.00 | 0.78 | 1SG | 1862 |
| ATOM | 1862 | SG | CYS | 227 | −4.056 | 14.738 | 20.885 | 1.00 | 0.78 | 1SG | 1863 |
| ATOM | 1863 | C | CYS | 227 | −5.012 | 13.631 | 24.739 | 1.00 | 0.78 | 1SG | 1864 |
| ATOM | 1864 | O | CYS | 227 | −4.212 | 13.939 | 25.620 | 1.00 | 0.78 | 1SG | 1865 |
| ATOM | 1865 | N | GLY | 228 | −5.870 | 12.602 | 24.862 | 1.00 | 0.64 | 1SG | 1866 |
| ATOM | 1866 | CA | GLY | 228 | −5.880 | 11.831 | 26.064 | 1.00 | 0.64 | 1SG | 1867 |
| ATOM | 1867 | C | GLY | 228 | −4.673 | 10.947 | 26.155 | 1.00 | 0.64 | 1SG | 1868 |
| ATOM | 1868 | O | GLY | 228 | −3.794 | 11.180 | 26.985 | 1.00 | 0.64 | 1SG | 1869 |
| ATOM | 1869 | N | THR | 229 | −4.583 | 9.929 | 25.268 | 1.00 | 0.68 | 1SG | 1870 |
| ATOM | 1870 | CA | THR | 229 | −3.562 | 8.948 | 25.501 | 1.00 | 0.68 | 1SG | 1871 |
| ATOM | 1871 | CB | THR | 229 | −4.134 | 7.577 | 25.729 | 1.00 | 0.68 | 1SG | 1872 |
| ATOM | 1872 | OG1 | THR | 229 | −4.828 | 7.131 | 24.575 | 1.00 | 0.68 | 1SG | 1873 |
| ATOM | 1873 | CG2 | THR | 229 | −5.094 | 7.637 | 26.929 | 1.00 | 0.68 | 1SG | 1874 |
| ATOM | 1874 | C | THR | 229 | −2.587 | 8.823 | 24.371 | 1.00 | 0.68 | 1SG | 1875 |
| ATOM | 1875 | O | THR | 229 | −2.954 | 8.767 | 23.199 | 1.00 | 0.68 | 1SG | 1876 |
| ATOM | 1876 | N | ASP | 230 | −1.290 | 8.752 | 24.737 | 1.00 | 0.77 | 1SG | 1877 |
| ATOM | 1877 | CA | ASP | 230 | −0.215 | 8.487 | 23.824 | 1.00 | 0.77 | 1SG | 1878 |
| ATOM | 1878 | CB | ASP | 230 | 1.100 | 9.178 | 24.242 | 1.00 | 0.77 | 1SG | 1879 |
| ATOM | 1879 | CG | ASP | 230 | 2.046 | 9.192 | 23.051 | 1.00 | 0.77 | 1SG | 1880 |
| ATOM | 1880 | OD1 | ASP | 230 | 1.902 | 8.297 | 22.176 | 1.00 | 0.77 | 1SG | 1881 |
| ATOM | 1881 | OD2 | ASP | 230 | 2.920 | 10.098 | 22.993 | 1.00 | 0.77 | 1SG | 1882 |
| ATOM | 1882 | C | ASP | 230 | −0.074 | 7.005 | 23.960 | 1.00 | 0.77 | 1SG | 1883 |
| ATOM | 1883 | O | ASP | 230 | −0.378 | 6.473 | 25.025 | 1.00 | 0.77 | 1SG | 1884 |
| ATOM | 1884 | N | PHE | 231 | 0.382 | 6.281 | 22.920 | 1.00 | 0.98 | 1SG | 1885 |
| ATOM | 1885 | CA | PHE | 231 | 0.245 | 4.859 | 23.066 | 1.00 | 0.98 | 1SG | 1886 |
| ATOM | 1886 | CB | PHE | 231 | 0.343 | 4.029 | 21.773 | 1.00 | 0.98 | 1SG | 1887 |
| ATOM | 1887 | CG | PHE | 231 | 1.640 | 4.233 | 21.093 | 1.00 | 0.98 | 1SG | 1888 |
| ATOM | 1888 | CD1 | PHE | 231 | 1.835 | 5.350 | 20.322 | 1.00 | 0.98 | 1SG | 1889 |
| ATOM | 1889 | CD2 | PHE | 231 | 2.639 | 3.296 | 21.212 | 1.00 | 0.98 | 1SG | 1890 |
| ATOM | 1890 | CE1 | PHE | 231 | 3.034 | 5.525 | 19.687 | 1.00 | 0.98 | 1SG | 1891 |
| ATOM | 1891 | CE2 | PHE | 231 | 3.841 | 3.468 | 20.575 | 1.00 | 0.98 | 1SG | 1892 |
| ATOM | 1892 | CZ | PHE | 231 | 4.033 | 4.589 | 19.808 | 1.00 | 0.98 | 1SG | 1893 |
| ATOM | 1893 | C | PHE | 231 | 1.037 | 4.261 | 24.183 | 1.00 | 0.98 | 1SG | 1894 |
| ATOM | 1894 | O | PHE | 231 | 2.182 | 4.587 | 24.484 | 1.00 | 0.98 | 1SG | 1895 |
| ATOM | 1895 | N | PRO | 232 | 0.290 | 3.395 | 24.815 | 1.00 | 1.14 | 1SG | 1896 |
| ATOM | 1896 | CA | PRO | 232 | 0.723 | 2.721 | 26.021 | 1.00 | 1.14 | 1SG | 1897 |
| ATOM | 1897 | CD | PRO | 232 | −1.121 | 3.751 | 24.887 | 1.00 | 1.14 | 1SG | 1898 |
| ATOM | 1898 | CB | PRO | 232 | −0.554 | 2.428 | 26.810 | 1.00 | 1.14 | 1SG | 1899 |
| ATOM | 1899 | CG | PRO | 232 | −1.555 | 3.480 | 26.327 | 1.00 | 1.14 | 1SG | 1900 |
| ATOM | 1900 | C | PRO | 232 | 1.581 | 1.480 | 25.999 | 1.00 | 1.14 | 1SG | 1901 |
| ATOM | 1901 | O | PRO | 232 | 1.931 | 1.048 | 27.093 | 1.00 | 1.14 | 1SG | 1902 |
| ATOM | 1902 | N | ILE | 233 | 1.939 | 0.829 | 24.874 | 1.00 | 1.05 | 1SG | 1903 |
| ATOM | 1903 | CA | ILE | 233 | 2.638 | −0.400 | 25.174 | 1.00 | 1.05 | 1SG | 1904 |
| ATOM | 1904 | CB | ILE | 233 | 2.691 | −1.445 | 24.086 | 1.00 | 1.05 | 1SG | 1905 |
| ATOM | 1905 | CG2 | ILE | 233 | 1.267 | −1.989 | 23.903 | 1.00 | 1.05 | 1SG | 1906 |
| ATOM | 1906 | CG1 | ILE | 233 | 3.410 | −0.969 | 22.823 | 1.00 | 1.05 | 1SG | 1907 |
| ATOM | 1907 | CD1 | ILE | 233 | 4.919 | −1.071 | 22.969 | 1.00 | 1.05 | 1SG | 1908 |
| ATOM | 1908 | C | ILE | 233 | 3.993 | −0.162 | 25.776 | 1.00 | 1.05 | 1SG | 1909 |
| ATOM | 1909 | O | ILE | 233 | 4.282 | −0.724 | 26.831 | 1.00 | 1.05 | 1SG | 1910 |
| ATOM | 1910 | N | LYS | 234 | 4.873 | 0.671 | 25.187 | 1.00 | 0.84 | 1SG | 1911 |
| ATOM | 1911 | CA | LYS | 234 | 6.051 | 0.862 | 25.979 | 1.00 | 0.84 | 1SG | 1912 |
| ATOM | 1912 | CB | LYS | 234 | 7.370 | 1.231 | 25.281 | 1.00 | 0.84 | 1SG | 1913 |
| ATOM | 1913 | CG | LYS | 234 | 8.156 | 0.038 | 24.729 | 1.00 | 0.84 | 1SG | 1914 |
| ATOM | 1914 | CD | LYS | 234 | 9.464 | 0.458 | 24.051 | 1.00 | 0.84 | 1SG | 1915 |
| ATOM | 1915 | CE | LYS | 234 | 10.449 | −0.695 | 23.816 | 1.00 | 0.84 | 1SG | 1916 |
| ATOM | 1916 | NZ | LYS | 234 | 11.762 | −0.183 | 23.349 | 1.00 | 0.84 | 1SG | 1917 |
| ATOM | 1917 | C | LYS | 234 | 5.737 | 1.931 | 26.960 | 1.00 | 0.84 | 1SG | 1918 |
| ATOM | 1918 | O | LYS | 234 | 4.845 | 1.724 | 27.775 | 1.00 | 0.84 | 1SG | 1919 |
| ATOM | 1919 | N | SER | 235 | 6.433 | 3.092 | 26.882 | 1.00 | 0.79 | 1SG | 1920 |
| ATOM | 1920 | CA | SER | 235 | 6.329 | 4.151 | 27.855 | 1.00 | 0.79 | 1SG | 1921 |
| ATOM | 1921 | CB | SER | 235 | 7.577 | 5.039 | 27.984 | 1.00 | 0.79 | 1SG | 1922 |
| ATOM | 1922 | OG | SER | 235 | 8.512 | 4.452 | 28.875 | 1.00 | 0.79 | 1SG | 1923 |
| ATOM | 1923 | C | SER | 235 | 5.197 | 5.075 | 27.559 | 1.00 | 0.79 | 1SG | 1924 |
| ATOM | 1924 | O | SER | 235 | 4.076 | 4.645 | 27.285 | 1.00 | 0.79 | 1SG | 1925 |
| ATOM | 1925 | N | ASN | 236 | 5.504 | 6.394 | 27.643 | 1.00 | 0.77 | 1SG | 1926 |
| ATOM | 1926 | CA | ASN | 236 | 4.582 | 7.469 | 27.398 | 1.00 | 0.77 | 1SG | 1927 |
| ATOM | 1927 | CB | ASN | 236 | 5.318 | 8.811 | 27.252 | 1.00 | 0.77 | 1SG | 1928 |
| ATOM | 1928 | CG | ASN | 236 | 4.291 | 9.921 | 27.316 | 1.00 | 0.77 | 1SG | 1929 |
| ATOM | 1929 | OD1 | ASN | 236 | 4.405 | 10.831 | 28.136 | 1.00 | 0.77 | 1SG | 1930 |
| ATOM | 1930 | ND2 | ASN | 236 | 3.266 | 9.845 | 26.426 | 1.00 | 0.77 | 1SG | 1931 |
| ATOM | 1931 | C | ASN | 236 | 3.998 | 7.097 | 26.086 | 1.00 | 0.77 | 1SG | 1932 |
| ATOM | 1932 | O | ASN | 236 | 2.804 | 7.227 | 25.822 | 1.00 | 0.77 | 1SG | 1933 |
| ATOM | 1933 | N | ALA | 237 | 4.896 | 6.629 | 25.218 | 1.00 | 0.69 | 1SG | 1934 |
| ATOM | 1934 | CA | ALA | 237 | 4.512 | 5.917 | 24.056 | 1.00 | 0.69 | 1SG | 1935 |
| ATOM | 1935 | CB | ALA | 237 | 4.466 | 6.698 | 22.744 | 1.00 | 0.69 | 1SG | 1936 |
| ATOM | 1936 | C | ALA | 237 | 5.589 | 4.918 | 23.983 | 1.00 | 0.69 | 1SG | 1937 |
| ATOM | 1937 | O | ALA | 237 | 6.573 | 5.043 | 24.711 | 1.00 | 0.69 | 1SG | 1938 |
| ATOM | 1938 | N | GLU | 238 | 5.414 | 3.871 | 23.168 | 1.00 | 0.73 | 1SG | 1939 |
| ATOM | 1939 | CA | GLU | 238 | 6.495 | 2.945 | 23.067 | 1.00 | 0.73 | 1SG | 1940 |
| ATOM | 1940 | CB | GLU | 238 | 6.212 | 1.849 | 22.021 | 1.00 | 0.73 | 1SG | 1941 |
| ATOM | 1941 | CG | GLU | 238 | 7.133 | 0.624 | 22.039 | 1.00 | 0.73 | 1SG | 1942 |
| ATOM | 1942 | CD | GLU | 238 | 8.444 | 0.899 | 21.314 | 1.00 | 0.73 | 1SG | 1943 |
| ATOM | 1943 | OE1 | GLU | 238 | 8.585 | 1.993 | 20.711 | 1.00 | 0.73 | 1SG | 1944 |
| ATOM | 1944 | OE2 | GLU | 238 | 9.328 | 0.003 | 21.344 | 1.00 | 0.73 | 1SG | 1945 |
| ATOM | 1945 | C | GLU | 238 | 7.595 | 3.775 | 22.533 | 1.00 | 0.73 | 1SG | 1946 |
| ATOM | 1946 | O | GLU | 238 | 8.726 | 3.785 | 23.017 | 1.00 | 0.73 | 1SG | 1947 |
| ATOM | 1947 | N | MET | 239 | 7.194 | 4.546 | 21.525 | 1.00 | 0.62 | 1SG | 1948 |
| ATOM | 1948 | CA | MET | 239 | 7.998 | 5.476 | 20.821 | 1.00 | 0.62 | 1SG | 1949 |
| ATOM | 1949 | CB | MET | 239 | 9.038 | 4.856 | 19.870 | 1.00 | 0.62 | 1SG | 1950 |
| ATOM | 1950 | CG | MET | 239 | 10.226 | 4.187 | 20.561 | 1.00 | 0.62 | 1SG | 1951 |
| ATOM | 1951 | SD | MET | 239 | 11.442 | 3.485 | 19.405 | 1.00 | 0.62 | 1SG | 1952 |
| ATOM | 1952 | CE | MET | 239 | 12.320 | 2.483 | 20.638 | 1.00 | 0.62 | 1SG | 1953 |
| ATOM | 1953 | C | MET | 239 | 7.001 | 6.144 | 19.962 | 1.00 | 0.62 | 1SG | 1954 |
| ATOM | 1954 | O | MET | 239 | 5.907 | 6.481 | 20.407 | 1.00 | 0.62 | 1SG | 1955 |
| ATOM | 1955 | N | VAL | 240 | 7.367 | 6.357 | 18.694 | 1.00 | 0.52 | 1SG | 1956 |
| ATOM | 1956 | CA | VAL | 240 | 6.408 | 6.904 | 17.799 | 1.00 | 0.52 | 1SG | 1957 |
| ATOM | 1957 | CB | VAL | 240 | 7.033 | 7.695 | 16.689 | 1.00 | 0.52 | 1SG | 1958 |
| ATOM | 1958 | CG1 | VAL | 240 | 5.920 | 8.236 | 15.780 | 1.00 | 0.52 | 1SG | 1959 |
| ATOM | 1959 | CG2 | VAL | 240 | 7.918 | 8.787 | 17.309 | 1.00 | 0.52 | 1SG | 1960 |
| ATOM | 1960 | C | VAL | 240 | 5.702 | 5.736 | 17.197 | 1.00 | 0.52 | 1SG | 1961 |
| ATOM | 1961 | O | VAL | 240 | 6.327 | 4.774 | 16.753 | 1.00 | 0.52 | 1SG | 1962 |
| ATOM | 1962 | N | GLN | 241 | 4.358 | 5.769 | 17.224 | 1.00 | 0.54 | 1SG | 1963 |
| ATOM | 1963 | CA | GLN | 241 | 3.595 | 4.713 | 16.635 | 1.00 | 0.54 | 1SG | 1964 |
| ATOM | 1964 | CB | GLN | 241 | 2.078 | 4.854 | 16.875 | 1.00 | 0.54 | 1SG | 1965 |
| ATOM | 1965 | CG | GLN | 241 | 1.443 | 6.070 | 16.199 | 1.00 | 0.54 | 1SG | 1966 |
| ATOM | 1966 | CD | GLN | 241 | 1.013 | 5.652 | 14.801 | 1.00 | 0.54 | 1SG | 1967 |
| ATOM | 1967 | OE1 | GLN | 241 | 0.581 | 6.456 | 13.982 | 1.00 | 0.54 | 1SG | 1968 |
| ATOM | 1968 | NE2 | GLN | 241 | 1.122 | 4.333 | 14.506 | 1.00 | 0.54 | 1SG | 1969 |
| ATOM | 1969 | C | GLN | 241 | 3.860 | 4.857 | 15.183 | 1.00 | 0.54 | 1SG | 1970 |
| ATOM | 1970 | O | GLN | 241 | 3.915 | 3.882 | 14.434 | 1.00 | 0.54 | 1SG | 1971 |
| ATOM | 1971 | N | ALA | 242 | 4.032 | 6.122 | 14.761 | 1.00 | 0.53 | 1SG | 1972 |
| ATOM | 1972 | CA | ALA | 242 | 4.304 | 6.407 | 13.390 | 1.00 | 0.53 | 1SG | 1973 |
| ATOM | 1973 | CB | ALA | 242 | 3.785 | 7.784 | 12.950 | 1.00 | 0.53 | 1SG | 1974 |
| ATOM | 1974 | C | ALA | 242 | 5.784 | 6.408 | 13.207 | 1.00 | 0.53 | 1SG | 1975 |
| ATOM | 1975 | O | ALA | 242 | 6.552 | 6.162 | 14.136 | 1.00 | 0.53 | 1SG | 1976 |
| ATOM | 1976 | N | LEU | 243 | 6.223 | 6.651 | 11.962 | 1.00 | 0.74 | 1SG | 1977 |
| ATOM | 1977 | CA | LEU | 243 | 7.627 | 6.686 | 11.703 | 1.00 | 0.74 | 1SG | 1978 |
| ATOM | 1978 | CB | LEU | 243 | 8.139 | 5.462 | 10.916 | 1.00 | 0.74 | 1SG | 1979 |
| ATOM | 1979 | CG | LEU | 243 | 7.402 | 5.184 | 9.590 | 1.00 | 0.74 | 1SG | 1980 |
| ATOM | 1980 | CD2 | LEU | 243 | 7.647 | 3.745 | 9.112 | 1.00 | 0.74 | 1SG | 1981 |
| ATOM | 1981 | CD1 | LEU | 243 | 7.719 | 6.232 | 8.511 | 1.00 | 0.74 | 1SG | 1982 |
| ATOM | 1982 | C | LEU | 243 | 7.892 | 7.918 | 10.914 | 1.00 | 0.74 | 1SG | 1983 |
| ATOM | 1983 | O | LEU | 243 | 7.009 | 8 426 | 10.224 | 1.00 | 0.74 | 1SG | 1984 |
| ATOM | 1984 | N | LYS | 244 | 9.127 | 8.434 | 11.019 | 1.00 | 0.97 | 1SG | 1985 |
| ATOM | 1985 | CA | LYS | 244 | 9.490 | 9.625 | 10.323 | 1.00 | 0.97 | 1SG | 1986 |
| ATOM | 1986 | CB | LYS | 244 | 10.875 | 10.139 | 10.763 | 1.00 | 0.97 | 1SG | 1987 |
| ATOM | 1987 | CG | LYS | 244 | 11.172 | 11.612 | 10.466 | 1.00 | 0.97 | 1SG | 1988 |
| ATOM | 1988 | CD | LYS | 244 | 11.229 | 11.961 | 8.983 | 1.00 | 0.97 | 1SG | 1989 |
| ATOM | 1989 | CE | LYS | 244 | 11.830 | 13.338 | 8.697 | 1.00 | 0.97 | 1SG | 1990 |
| ATOM | 1990 | NZ | LYS | 244 | 11.949 | 13.538 | 7.234 | 1.00 | 0.97 | 1SG | 1991 |
| ATOM | 1991 | C | LYS | 244 | 9.523 | 9.269 | 8.878 | 1.00 | 0.97 | 1SG | 1992 |
| ATOM | 1992 | O | LYS | 244 | 9.865 | 8.149 | 8.504 | 1.00 | 0.97 | 1SG | 1993 |
| ATOM | 1993 | N | MET | 245 | 9.123 | 10 225 | 8.028 | 1.00 | 1.00 | 1SG | 1994 |
| ATOM | 1994 | CA | MET | 245 | 9.054 | 10.017 | 6.617 | 1.00 | 1.00 | 1SG | 1995 |
| ATOM | 1995 | CB | MET | 245 | 8.392 | 11.200 | 5.900 | 1.00 | 1.00 | 1SG | 1996 |
| ATOM | 1996 | CG | MET | 245 | 8.986 | 12.547 | 6.310 | 1.00 | 1.00 | 1SG | 1997 |
| ATOM | 1997 | SD | MET | 245 | 8.538 | 13.112 | 7.981 | 1.00 | 1.00 | 1SG | 1998 |
| ATOM | 1998 | CE | MET | 245 | 9.359 | 14.725 | 7.826 | 1.00 | 1.00 | 1SG | 1999 |
| ATOM | 1999 | C | MET | 245 | 10.422 | 9.816 | 6.048 | 1.00 | 1.00 | 1SG | 2000 |
| ATOM | 2000 | O | MET | 245 | 10.601 | 8.987 | 5.158 | 1.00 | 1.00 | 1SG | 2001 |
| ATOM | 2001 | N | LEU | 246 | 11.426 | 10.566 | 6.542 | 1.00 | 0.74 | 1SG | 2002 |
| ATOM | 2002 | CA | LEU | 246 | 12.743 | 10.477 | 5.979 | 1.00 | 0.74 | 1SG | 2003 |
| ATOM | 2003 | CB | LEU | 246 | 13.279 | 9.047 | 5.750 | 1.00 | 0.74 | 1SG | 2004 |
| ATOM | 2004 | CG | LEU | 246 | 13.723 | 8.301 | 7.023 | 1.00 | 0.74 | 1SG | 2005 |
| ATOM | 2005 | CD2 | LEU | 246 | 14.533 | 7.043 | 6.670 | 1.00 | 0.74 | 1SG | 2006 |
| ATOM | 2006 | CD1 | LEU | 246 | 12.545 | 8.004 | 7.958 | 1.00 | 0.74 | 1SG | 2007 |
| ATOM | 2007 | C | LEU | 246 | 12.668 | 11.149 | 4.655 | 1.00 | 0.74 | 1SG | 2008 |
| ATOM | 2008 | O | LEU | 246 | 12.993 | 12.327 | 4.515 | 1.00 | 0.74 | 1SG | 2009 |
| ATOM | 2009 | N | ASN | 247 | 12.212 | 10.392 | 3.645 | 1.00 | 0.37 | 1SG | 2010 |
| ATOM | 2010 | CA | ASN | 247 | 12.076 | 10.908 | 2.322 | 1.00 | 0.37 | 1SG | 2011 |
| ATOM | 2011 | CB | ASN | 247 | 11.539 | 9.868 | 1.326 | 1.00 | 0.37 | 1SG | 2012 |
| ATOM | 2012 | CG | ASN | 247 | 12.625 | 8.822 | 1.118 | 1.00 | 0.37 | 1SG | 2013 |
| ATOM | 2013 | OD1 | ASN | 247 | 13.799 | 9.069 | 1.388 | 1.00 | 0.37 | 1SG | 2014 |
| ATOM | 2014 | ND2 | ASN | 247 | 12.227 | 7.623 | 0.614 | 1.00 | 0.37 | 1SG | 2015 |
| ATOM | 2015 | C | ASN | 247 | 11.098 | 12.032 | 2.382 | 1.00 | 0.37 | 1SG | 2016 |
| ATOM | 2016 | O | ASN | 247 | 11.166 | 12.951 | 1.567 | 1.00 | 0.37 | 1SG | 2017 |
| ATOM | 2017 | N | GLY | 248 | 10.150 | 11.986 | 3.343 | 1.00 | 0.30 | 1SG | 2018 |
| ATOM | 2018 | CA | GLY | 248 | 9.186 | 13.046 | 3.410 | 1.00 | 0.30 | 1SG | 2019 |
| ATOM | 2019 | C | GLY | 248 | 9.825 | 14.239 | 4.049 | 1.00 | 0.30 | 1SG | 2020 |
| ATOM | 2020 | O | GLY | 248 | 9.240 | 14.889 | 4.915 | 1.00 | 0.30 | 1SG | 2021 |
| ATOM | 2021 | N | ARG | 249 | 11.049 | 14.575 | 3.611 | 1.00 | 0.40 | 1SG | 2022 |
| ATOM | 2022 | CA | ARG | 249 | 11.713 | 15.726 | 4.134 | 1.00 | 0.40 | 1SG | 2023 |
| ATOM | 2023 | CB | ARG | 249 | 13.142 | 15.895 | 3.588 | 1.00 | 0.40 | 1SG | 2024 |
| ATOM | 2024 | CG | ARG | 249 | 14.100 | 14.810 | 4.086 | 1.00 | 0.40 | 1SG | 2025 |
| ATOM | 2025 | CD | ARG | 249 | 14.918 | 15.223 | 5.312 | 1.00 | 0.40 | 1SG | 2026 |
| ATOM | 2026 | NE | ARG | 249 | 15.978 | 16.164 | 4.852 | 1.00 | 0.40 | 1SG | 2027 |
| ATOM | 2027 | CZ | ARG | 249 | 17.192 | 15.672 | 4.466 | 1.00 | 0.40 | 1SG | 2028 |
| ATOM | 2028 | NH1 | ARG | 249 | 17.428 | 14.329 | 4.514 | 1.00 | 0.40 | 1SG | 2029 |
| ATOM | 2029 | NH2 | ARG | 249 | 18.173 | 16.514 | 4.032 | 1.00 | 0.40 | 1SG | 2030 |
| ATOM | 2030 | C | ARG | 249 | 10.908 | 16.903 | 3.699 | 1.00 | 0.40 | 1SG | 2031 |
| ATOM | 2031 | O | ARG | 249 | 10.712 | 17.852 | 4.456 | 1.00 | 0.40 | 1SG | 2032 |
| ATOM | 2032 | N | ASN | 250 | 10.399 | 16.851 | 2.454 | 1.00 | 0.43 | 1SG | 2033 |
| ATOM | 2033 | CA | ASN | 250 | 9.635 | 17.949 | 1.947 | 1.00 | 0.43 | 1SG | 2034 |
| ATOM | 2034 | CB | ASN | 250 | 9.775 | 18.146 | 0.427 | 1.00 | 0.43 | 1SG | 2035 |
| ATOM | 2035 | CG | ASN | 250 | 11.207 | 18.574 | 0.132 | 1.00 | 0.43 | 1SG | 2036 |
| ATOM | 2036 | OD1 | ASN | 250 | 11.749 | 19.471 | 0.775 | 1.00 | 0.43 | 1SG | 2037 |
| ATOM | 2037 | ND2 | ASN | 250 | 11.843 | 17.907 | −0.868 | 1.00 | 0.43 | 1SG | 2038 |
| ATOM | 2038 | C | ASN | 250 | 8.197 | 17.673 | 2.237 | 1.00 | 0.43 | 1SG | 2039 |
| ATOM | 2039 | O | ASN | 250 | 7.685 | 16.588 | 1.960 | 1.00 | 0.43 | 1SG | 2040 |
| ATOM | 2040 | N | SER | 251 | 7.519 | 18.669 | 2.836 | 1.00 | 0.70 | 1SG | 2041 |
| ATOM | 2041 | CA | SER | 251 | 6.133 | 18.560 | 3.176 | 1.00 | 0.70 | 1SG | 2042 |
| ATOM | 2042 | CB | SER | 251 | 5.903 | 18.035 | 4.605 | 1.00 | 0.70 | 1SG | 2043 |
| ATOM | 2043 | OG | SER | 251 | 4.517 | 17.843 | 4.846 | 1.00 | 0.70 | 1SG | 2044 |
| ATOM | 2044 | C | SER | 251 | 5.588 | 19.952 | 3.089 | 1.00 | 0.70 | 1SG | 2045 |
| ATOM | 2045 | O | SER | 251 | 6.250 | 20.849 | 2.569 | 1.00 | 0.70 | 1SG | 2046 |
| ATOM | 2046 | N | MET | 252 | 4.347 | 20.170 | 3.567 | 1.00 | 1.01 | 1SG | 2047 |
| ATOM | 2047 | CA | MET | 252 | 3.784 | 21.487 | 3.494 | 1.00 | 1.01 | 1SG | 2048 |
| ATOM | 2048 | CB | MET | 252 | 2.341 | 21.567 | 4.024 | 1.00 | 1.01 | 1SG | 2049 |
| ATOM | 2049 | CG | MET | 252 | 2.214 | 21.299 | 5.525 | 1.00 | 1.01 | 1SG | 2050 |
| ATOM | 2050 | SD | MET | 252 | 2.485 | 19.568 | 6.012 | 1.00 | 1.01 | 1SG | 2051 |
| ATOM | 2051 | CE | MET | 252 | 2.399 | 19.895 | 7.794 | 1.00 | 1.01 | 1SG | 2052 |
| ATOM | 2052 | C | MET | 252 | 4 618 | 22.399 | 4.331 | 1.00 | 1.01 | 1SG | 2053 |
| ATOM | 2053 | O | MET | 252 | 4.992 | 23.486 | 3.894 | 1.00 | 1.01 | 1SG | 2054 |
| ATOM | 2054 | N | GLU | 253 | 4.961 | 21.966 | 5.558 | 1.00 | 0.86 | 1SG | 2055 |
| ATOM | 2055 | CA | GLU | 253 | 5.725 | 22.821 | 6.411 | 1.00 | 0.86 | 1SG | 2056 |
| ATOM | 2056 | CB | GLU | 253 | 5.572 | 22.525 | 7.915 | 1.00 | 0.86 | 1SG | 2057 |
| ATOM | 2057 | CG | GLU | 253 | 6.055 | 21.137 | 8.331 | 1.00 | 0.86 | 1SG | 2058 |
| ATOM | 2058 | CD | GLU | 253 | 5.894 | 21.031 | 9.841 | 1.00 | 0.86 | 1SG | 2059 |
| ATOM | 2059 | OE1 | GLU | 253 | 5.402 | 22.018 | 10.452 | 1.00 | 0.86 | 1SG | 2060 |
| ATOM | 2060 | OE2 | GLU | 253 | 6.262 | 19.966 | 10.403 | 1.00 | 0.86 | 1SG | 2061 |
| ATOM | 2061 | C | GLU | 253 | 7 165 | 22.670 | 6.061 | 1.00 | 0.86 | 1SG | 2062 |
| ATOM | 2062 | O | GLU | 253 | 7.526 | 21.943 | 5.136 | 1.00 | 0.86 | 1SG | 2063 |
| ATOM | 2063 | N | SER | 254 | 8.028 | 23.388 | 6.805 | 1.00 | 0.54 | 1SG | 2064 |
| ATOM | 2064 | CA | SER | 254 | 9.435 | 23.385 | 6.541 | 1.00 | 0.54 | 1SG | 2065 |
| ATOM | 2065 | CB | SER | 254 | 10.190 | 24 535 | 7.224 | 1.00 | 0.54 | 1SG | 2066 |
| ATOM | 2066 | OG | SER | 254 | 11.572 | 24.466 | 6.911 | 1.00 | 0.54 | 1SG | 2067 |
| ATOM | 2067 | C | SER | 254 | 10.037 | 22.098 | 7.012 | 1.00 | 0.54 | 1SG | 2068 |
| ATOM | 2068 | O | SER | 254 | 9.506 | 21.413 | 7.885 | 1.00 | 0.54 | 1SG | 2069 |
| ATOM | 2069 | N | GLU | 255 | 11.179 | 21.754 | 6.389 | 1.00 | 0.38 | 1SG | 2070 |
| ATOM | 2070 | CA | GLU | 255 | 11.968 | 20.577 | 6.610 | 1.00 | 0.38 | 1SG | 2071 |
| ATOM | 2071 | CB | GLU | 255 | 13.058 | 20.404 | 5.540 | 1.00 | 0.38 | 1SG | 2072 |
| ATOM | 2072 | CG | GLU | 255 | 12.501 | 20.134 | 4.139 | 1.00 | 0.38 | 1SG | 2073 |
| ATOM | 2073 | CD | GLU | 255 | 13.674 | 19.996 | 3.178 | 1.00 | 0.38 | 1SG | 2074 |
| ATOM | 2074 | OE1 | GLU | 255 | 14.534 | 20.917 | 3.160 | 1.00 | 0.38 | 1SG | 2075 |
| ATOM | 2075 | OE2 | GLU | 255 | 13.727 | 18.968 | 2.450 | 1.00 | 0.38 | 1SG | 2076 |
| ATOM | 2076 | C | GLU | 255 | 12.652 | 20.636 | 7.941 | 1.00 | 0.38 | 1SG | 2077 |
| ATOM | 2077 | O | GLU | 255 | 12.934 | 19.601 | 8.542 | 1.00 | 0.38 | 1SG | 2078 |
| ATOM | 2078 | N | VAL | 256 | 12.946 | 21.851 | 8.441 | 1.00 | 0.39 | 1SG | 2079 |
| ATOM | 2079 | CA | VAL | 256 | 13.744 | 21.951 | 9.628 | 1.00 | 0.39 | 1SG | 2080 |
| ATOM | 2080 | CB | VAL | 256 | 14.050 | 23.373 | 10.044 | 1.00 | 0.39 | 1SG | 2081 |
| ATOM | 2081 | CG1 | VAL | 256 | 12.763 | 24.205 | 10.157 | 1.00 | 0.39 | 1SG | 2082 |
| ATOM | 2082 | CG2 | VAL | 256 | 14.854 | 23.318 | 11.352 | 1.00 | 0.39 | 1SG | 2083 |
| ATOM | 2083 | C | VAL | 256 | 13.165 | 21.176 | 10.778 | 1.00 | 0.39 | 1SG | 2084 |
| ATOM | 2084 | O | VAL | 256 | 13.938 | 20.461 | 11.414 | 1.00 | 0.39 | 1SG | 2085 |
| ATOM | 2085 | N | PRO | 257 | 11.903 | 21.208 | 11.128 | 1.00 | 0.32 | 1SG | 2086 |
| ATOM | 2086 | CA | PRO | 257 | 11.439 | 20.447 | 12.253 | 1.00 | 0.32 | 1SG | 2087 |
| ATOM | 2087 | CD | PRO | 257 | 10.832 | 21.906 | 10.433 | 1.00 | 0.32 | 1SG | 2088 |
| ATOM | 2088 | CB | PRO | 257 | 9.954 | 20.798 | 12.406 | 1.00 | 0.32 | 1SG | 2089 |
| ATOM | 2089 | CG | PRO | 257 | 9.534 | 21.324 | 11.018 | 1.00 | 0.32 | 1SG | 2090 |
| ATOM | 2090 | C | PRO | 257 | 11.736 | 18.979 | 12.105 | 1.00 | 0.32 | 1SG | 2091 |
| ATOM | 2091 | O | PRO | 257 | 12.021 | 18.365 | 13.131 | 1.00 | 0.32 | 1SG | 2092 |
| ATOM | 2092 | N | PRO | 258 | 11.658 | 18.348 | 10.966 | 1.00 | 0.35 | 1SG | 2093 |
| ATOM | 2093 | CA | PRO | 258 | 12.053 | 16.970 | 10.939 | 1.00 | 0.35 | 1SG | 2094 |
| ATOM | 2094 | CD | PRO | 258 | 10.637 | 18.629 | 9.973 | 1.00 | 0.35 | 1SG | 2095 |
| ATOM | 2095 | CB | PRO | 258 | 11.347 | 16.337 | 9.736 | 1.00 | 0.35 | 1SG | 2096 |
| ATOM | 2096 | CG | PRO | 258 | 10.818 | 17.528 | 8.920 | 1.00 | 0.35 | 1SG | 2097 |
| ATOM | 2097 | C | PRO | 258 | 13.540 | 16.820 | 10.902 | 1.00 | 0.35 | 1SG | 2098 |
| ATOM | 2098 | O | PRO | 258 | 14.044 | 15.783 | 11.329 | 1.00 | 0.35 | 1SG | 2099 |
| ATOM | 2099 | N | LYS | 259 | 14.251 | 17.834 | 10.375 | 1.00 | 0.52 | 1SG | 2100 |
| ATOM | 2100 | CA | LYS | 259 | 15.668 | 17.753 | 10.159 | 1.00 | 0.52 | 1SG | 2101 |
| ATOM | 2101 | CB | LYS | 259 | 16.204 | 18.876 | 9.255 | 1.00 | 0.52 | 1SG | 2102 |
| ATOM | 2102 | CG | LYS | 259 | 15.802 | 18.750 | 7.785 | 1.00 | 0.52 | 1SG | 2103 |
| ATOM | 2103 | CD | LYS | 259 | 16.123 | 20.002 | 6.965 | 1.00 | 0.52 | 1SG | 2104 |
| ATOM | 2104 | CE | LYS | 259 | 15.945 | 19.816 | 5.458 | 1.00 | 0.52 | 1SG | 2105 |
| ATOM | 2105 | NZ | LYS | 259 | 16.977 | 18.901 | 4.939 | 1.00 | 0.52 | 1SG | 2106 |
| ATOM | 2106 | C | LYS | 259 | 16.503 | 17.795 | 11.403 | 1.00 | 0.52 | 1SG | 2107 |
| ATOM | 2107 | O | LYS | 259 | 17.428 | 16.997 | 11.547 | 1.00 | 0.52 | 1SG | 2108 |
| ATOM | 2108 | N | HIS | 260 | 16.204 | 18.698 | 12.355 | 1.00 | 0.64 | 1SG | 2109 |
| ATOM | 2109 | CA | HIS | 260 | 17.198 | 18.914 | 13.370 | 1.00 | 0.64 | 1SG | 2110 |
| ATOM | 2110 | ND1 | HIS | 260 | 19.371 | 21.390 | 12.699 | 1.00 | 0.64 | 1SG | 2111 |
| ATOM | 2111 | CG | HIS | 260 | 18.005 | 21.231 | 12.776 | 1.00 | 0.64 | 1SG | 2112 |
| ATOM | 2112 | CB | HIS | 260 | 17.323 | 20.383 | 13.810 | 1.00 | 0.64 | 1SG | 2113 |
| ATOM | 2113 | NE2 | HIS | 260 | 18.499 | 22.585 | 11.039 | 1.00 | 0.64 | 1SG | 2114 |
| ATOM | 2114 | CD2 | HIS | 260 | 17.489 | 21.967 | 11.755 | 1.00 | 0.64 | 1SG | 2115 |
| ATOM | 2115 | CE1 | HIS | 260 | 19.611 | 22.208 | 11.643 | 1.00 | 0.64 | 1SG | 2116 |
| ATOM | 2116 | C | HIS | 260 | 17.055 | 18.077 | 14.595 | 1.00 | 0.64 | 1SG | 2117 |
| ATOM | 2117 | O | HIS | 260 | 16.021 | 17.472 | 14.877 | 1.00 | 0.64 | 1SG | 2118 |
| ATOM | 2118 | N | LYS | 261 | 18.184 | 18.023 | 15.334 | 1.00 | 0.79 | 1SG | 2119 |
| ATOM | 2119 | CA | LYS | 261 | 18.335 | 17.349 | 16.587 | 1.00 | 0.79 | 1SG | 2120 |
| ATOM | 2120 | CB | LYS | 261 | 19.794 | 17.324 | 17.074 | 1.00 | 0.79 | 1SG | 2121 |
| ATOM | 2121 | CG | LYS | 261 | 20.719 | 16.457 | 16.219 | 1.00 | 0.79 | 1SG | 2122 |
| ATOM | 2122 | CD | LYS | 261 | 20.360 | 14.970 | 16.241 | 1.00 | 0.79 | 1SG | 2123 |
| ATOM | 2123 | CE | LYS | 261 | 20.696 | 14.288 | 17.568 | 1.00 | 0.79 | 1SG | 2124 |
| ATOM | 2124 | NZ | LYS | 261 | 22.152 | 14.361 | 17.820 | 1.00 | 0.79 | 1SG | 2125 |
| ATOM | 2125 | C | LYS | 261 | 17.557 | 18.128 | 17.592 | 1.00 | 0.79 | 1SG | 2126 |
| ATOM | 2126 | O | LYS | 261 | 16.946 | 17.560 | 18.496 | 1.00 | 0.79 | 1SG | 2127 |
| ATOM | 2127 | N | GLU | 262 | 17.584 | 19.469 | 17.472 | 1.00 | 0.88 | 1SG | 2128 |
| ATOM | 2128 | CA | GLU | 262 | 16.817 | 20.262 | 18.380 | 1.00 | 0.88 | 1SG | 2129 |
| ATOM | 2129 | CB | GLU | 262 | 16.952 | 21.773 | 18.131 | 1.00 | 0.88 | 1SG | 2130 |
| ATOM | 2130 | CG | GLU | 262 | 16.525 | 22.212 | 16.729 | 1.00 | 0.88 | 1SG | 2131 |
| ATOM | 2131 | CD | GLU | 262 | 16.713 | 23.719 | 16.634 | 1.00 | 0.88 | 1SG | 2132 |
| ATOM | 2132 | OE1 | GLU | 262 | 17.074 | 24.336 | 17.670 | 1.00 | 0.88 | 1SG | 2133 |
| ATOM | 2133 | OE2 | GLU | 262 | 16.495 | 24.273 | 15.523 | 1.00 | 0.88 | 1SG | 2134 |
| ATOM | 2134 | C | GLU | 262 | 15.405 | 19.867 | 18.133 | 1.00 | 0.88 | 1SG | 2135 |
| ATOM | 2135 | O | GLU | 262 | 14.650 | 19.585 | 19.062 | 1.00 | 0.88 | 1SG | 2136 |
| ATOM | 2136 | N | THR | 263 | 15.022 | 19.822 | 16.844 | 1.00 | 0.94 | 1SG | 2137 |
| ATOM | 2137 | CA | THR | 263 | 13.720 | 19.355 | 16.494 | 1.00 | 0.94 | 1SG | 2138 |
| ATOM | 2138 | CB | THR | 263 | 13.324 | 19.737 | 15.102 | 1.00 | 0.94 | 1SG | 2139 |
| ATOM | 2139 | OG1 | THR | 263 | 14.237 | 19.191 | 14.162 | 1.00 | 0.94 | 1SG | 2140 |
| ATOM | 2140 | CG2 | THR | 263 | 13.307 | 21.271 | 14.996 | 1.00 | 0.94 | 1SG | 2141 |
| ATOM | 2141 | C | THR | 263 | 13.832 | 17.872 | 16.603 | 1.00 | 0.94 | 1SG | 2142 |
| ATOM | 2142 | O | THR | 263 | 14.831 | 17.379 | 17.120 | 1.00 | 0.94 | 1SG | 2143 |
| ATOM | 2143 | N | ARG | 264 | 12.843 | 17.081 | 16.152 | 1.00 | 0.93 | 1SG | 2144 |
| ATOM | 2144 | CA | ARG | 264 | 13.129 | 15.703 | 16.420 | 1.00 | 0.93 | 1SG | 2145 |
| ATOM | 2145 | CB | ARG | 264 | 12.980 | 15.360 | 17.911 | 1.00 | 0.93 | 1SG | 2146 |
| ATOM | 2146 | CG | ARG | 264 | 11.567 | 15.614 | 18.446 | 1.00 | 0.93 | 1SG | 2147 |
| ATOM | 2147 | CD | ARG | 264 | 11.320 | 17.070 | 18.848 | 1.00 | 0.93 | 1SG | 2148 |
| ATOM | 2148 | NE | ARG | 264 | 9.917 | 17.175 | 19.344 | 1.00 | 0.93 | 1SG | 2149 |
| ATOM | 2149 | CZ | ARG | 264 | 8.914 | 17.537 | 18.492 | 1.00 | 0.93 | 1SG | 2150 |
| ATOM | 2150 | NH1 | ARG | 264 | 9.195 | 17.816 | 17.185 | 1.00 | 0.93 | 1SG | 2151 |
| ATOM | 2151 | NH2 | ARG | 264 | 7.631 | 17.629 | 18.949 | 1.00 | 0.93 | 1SG | 2152 |
| ATOM | 2152 | C | ARG | 264 | 12.193 | 14.797 | 15.700 | 1.00 | 0.93 | 1SG | 2153 |
| ATOM | 2153 | O | ARG | 264 | 11.869 | 13.730 | 16.221 | 1.00 | 0.93 | 1SG | 2154 |
| ATOM | 2154 | N | TRP | 265 | 11.724 | 15.171 | 14.498 | 1.00 | 0.87 | 1SG | 2155 |
| ATOM | 2155 | CA | TRP | 265 | 10.871 | 14.233 | 13.833 | 1.00 | 0.87 | 1SG | 2156 |
| ATOM | 2156 | CB | TRP | 265 | 10.402 | 14.727 | 12.453 | 1.00 | 0.87 | 1SG | 2157 |
| ATOM | 2157 | CG | TRP | 265 | 9.409 | 15.864 | 12.477 | 1.00 | 0.87 | 1SG | 2158 |
| ATOM | 2158 | CD2 | TRP | 265 | 8.070 | 15.770 | 11.965 | 1.00 | 0.87 | 1SG | 2159 |
| ATOM | 2159 | CD1 | TRP | 265 | 9.569 | 17.149 | 12.905 | 1.00 | 0.87 | 1SG | 2160 |
| ATOM | 2160 | NE1 | TRP | 265 | 8.412 | 17.861 | 12.697 | 1.00 | 0.87 | 1SG | 2161 |
| ATOM | 2161 | CE2 | TRP | 265 | 7.482 | 17.025 | 12.117 | 1.00 | 0.87 | 1SG | 2162 |
| ATOM | 2162 | CE3 | TRP | 265 | 7.391 | 14.726 | 11.407 | 1.00 | 0.87 | 1SG | 2163 |
| ATOM | 2163 | CZ2 | TRP | 265 | 6.198 | 17.255 | 11.713 | 1.00 | 0.87 | 1SG | 2164 |
| ATOM | 2164 | CZ3 | TRP | 265 | 6.094 | 14.959 | 11.007 | 1.00 | 0.87 | 1SG | 2165 |
| ATOM | 2165 | CH2 | TRP | 265 | 5.510 | 16.199 | 11.157 | 1.00 | 0.87 | 1SG | 2166 |
| ATOM | 2166 | C | TRP | 265 | 11.697 | 13.014 | 13.586 | 1.00 | 0.87 | 1SG | 2167 |
| ATOM | 2167 | O | TRP | 265 | 11.371 | 11.915 | 14.033 | 1.00 | 0.87 | 1SG | 2168 |
| ATOM | 2168 | N | LYS | 266 | 12.821 | 13.206 | 12.874 | 1.00 | 1.04 | 1SG | 2169 |
| ATOM | 2169 | CA | LYS | 266 | 13.740 | 12.146 | 12.599 | 1.00 | 1.04 | 1SG | 2170 |
| ATOM | 2170 | CB | LYS | 266 | 14.785 | 12.540 | 11.543 | 1.00 | 1.04 | 1SG | 2171 |
| ATOM | 2171 | CG | LYS | 266 | 15.691 | 13.689 | 11.991 | 1.00 | 1.04 | 1SG | 2172 |
| ATOM | 2172 | CD | LYS | 266 | 16.862 | 13.955 | 11.043 | 1.00 | 1.04 | 1SG | 2173 |
| ATOM | 2173 | CE | LYS | 266 | 17.886 | 12.821 | 10.997 | 1.00 | 1.04 | 1SG | 2174 |
| ATOM | 2174 | NZ | LYS | 266 | 18.971 | 13.160 | 10.051 | 1.00 | 1.04 | 1SG | 2175 |
| ATOM | 2175 | C | LYS | 266 | 14.472 | 11.831 | 13.863 | 1.00 | 1.04 | 1SG | 2176 |
| ATOM | 2176 | O | LYS | 266 | 14.732 | 10.671 | 14.173 | 1.00 | 1.04 | 1SG | 2177 |
| ATOM | 2177 | N | TYR | 267 | 14.773 | 12.895 | 14.635 | 1.00 | 1.18 | 1SG | 2178 |
| ATOM | 2178 | CA | TYR | 267 | 15.609 | 12.894 | 15.803 | 1.00 | 1.18 | 1SG | 2179 |
| ATOM | 2179 | CB | TYR | 267 | 14.935 | 12.669 | 17.162 | 1.00 | 1.18 | 1SG | 2180 |
| ATOM | 2180 | CG | TYR | 267 | 16.034 | 13.021 | 18.107 | 1.00 | 1.18 | 1SG | 2181 |
| ATOM | 2181 | CD1 | TYR | 267 | 16.261 | 14.339 | 18.432 | 1.00 | 1.18 | 1SG | 2182 |
| ATOM | 2182 | CD2 | TYR | 267 | 16.853 | 12.054 | 18.642 | 1.00 | 1.18 | 1SG | 2183 |
| ATOM | 2183 | CE1 | TYR | 267 | 17.278 | 14.690 | 19.289 | 1.00 | 1.18 | 1SG | 2184 |
| ATOM | 2184 | CE2 | TYR | 267 | 17.871 | 12.402 | 19.499 | 1.00 | 1.18 | 1SG | 2185 |
| ATOM | 2185 | CZ | TYR | 267 | 18.086 | 13.718 | 19.825 | 1.00 | 1.18 | 1SG | 2186 |
| ATOM | 2186 | OH | TYR | 267 | 19.133 | 14.069 | 20.704 | 1.00 | 1.18 | 1SG | 2187 |
| ATOM | 2187 | C | TYR | 267 | 16.687 | 11.883 | 15.639 | 1.00 | 1.18 | 1SG | 2188 |
| ATOM | 2188 | O | TYR | 267 | 16.539 | 10.720 | 16.021 | 1.00 | 1.18 | 1SG | 2189 |
| ATOM | 2189 | N | HIS | 268 | 17.810 | 12.345 | 15.054 | 1.00 | 1.03 | 1SG | 2190 |
| ATOM | 2190 | CA | HIS | 268 | 18.932 | 11.513 | 14.756 | 1.00 | 1.03 | 1SG | 2191 |
| ATOM | 2191 | ND1 | HIS | 268 | 22.139 | 10.894 | 13.994 | 1.00 | 1.03 | 1SG | 2192 |
| ATOM | 2192 | CG | HIS | 268 | 21.020 | 11.293 | 13.298 | 1.00 | 1.03 | 1SG | 2193 |
| ATOM | 2193 | CB | HIS | 268 | 19.961 | 12.207 | 13.843 | 1.00 | 1.03 | 1SG | 2194 |
| ATOM | 2194 | NE2 | HIS | 268 | 22.272 | 9.947 | 11.986 | 1.00 | 1.03 | 1SG | 2195 |
| ATOM | 2195 | CD2 | HIS | 268 | 21.117 | 10.705 | 12.074 | 1.00 | 1.03 | 1SG | 2196 |
| ATOM | 2196 | CE1 | HIS | 268 | 22.853 | 10.092 | 13.164 | 1.00 | 1.03 | 1SG | 2197 |
| ATOM | 2197 | C | HIS | 268 | 19.613 | 11.171 | 16.036 | 1.00 | 1.03 | 1SG | 2198 |
| ATOM | 2198 | O | HIS | 268 | 20.590 | 11.803 | 16.432 | 1.00 | 1.03 | 1SG | 2199 |
| ATOM | 2199 | N | PHE | 269 | 19.086 | 10.142 | 16.717 | 1.00 | 0.91 | 1SG | 2200 |
| ATOM | 2200 | CA | PHE | 269 | 19.668 | 9.625 | 17.915 | 1.00 | 0.91 | 1SG | 2201 |
| ATOM | 2201 | CB | PHE | 269 | 18.684 | 9.602 | 19.101 | 1.00 | 0.91 | 1SG | 2202 |
| ATOM | 2202 | CG | PHE | 269 | 19.429 | 9.247 | 20.338 | 1.00 | 0.91 | 1SG | 2203 |
| ATOM | 2203 | CD1 | PHE | 269 | 20.368 | 10.095 | 20.880 | 1.00 | 0.91 | 1SG | 2204 |
| ATOM | 2204 | CD2 | PHE | 269 | 19.152 | 8.080 | 20.989 | 1.00 | 0.91 | 1SG | 2205 |
| ATOM | 2205 | CE1 | PHE | 269 | 21.061 | 9.780 | 22.026 | 1.00 | 0.91 | 1SG | 2206 |
| ATOM | 2206 | CE2 | PHE | 269 | 19.848 | 7.778 | 22.127 | 1.00 | 0.91 | 1SG | 2207 |
| ATOM | 2207 | CZ | PHE | 269 | 20.805 | 8.598 | 22.663 | 1.00 | 0.91 | 1SG | 2208 |
| ATOM | 2208 | C | PHE | 269 | 20.000 | 8.233 | 17.508 | 1.00 | 0.91 | 1SG | 2209 |
| ATOM | 2209 | O | PHE | 269 | 20.457 | 8.031 | 16.383 | 1.00 | 0.91 | 1SG | 2210 |
| ATOM | 2210 | N | GLU | 270 | 19.836 | 7.239 | 18.397 | 1.00 | 0.79 | 1SG | 2211 |
| ATOM | 2211 | CA | GLU | 270 | 20.050 | 5.908 | 17.929 | 1.00 | 0.79 | 1SG | 2212 |
| ATOM | 2212 | CB | GLU | 270 | 19.786 | 4.844 | 19.006 | 1.00 | 0.79 | 1SG | 2213 |
| ATOM | 2213 | CG | GLU | 270 | 20.157 | 3.427 | 18.563 | 1.00 | 0.79 | 1SG | 2214 |
| ATOM | 2214 | CD | GLU | 270 | 21.677 | 3.331 | 18.532 | 1.00 | 0.79 | 1SG | 2215 |
| ATOM | 2215 | OE1 | GLU | 270 | 22.337 | 4.404 | 18.526 | 1.00 | 0.79 | 1SG | 2216 |
| ATOM | 2216 | OE2 | GLU | 270 | 22.197 | 2.183 | 18.512 | 1.00 | 0.79 | 1SG | 2217 |
| ATOM | 2217 | C | GLU | 270 | 19.031 | 5.748 | 16.855 | 1.00 | 0.79 | 1SG | 2218 |
| ATOM | 2218 | O | GLU | 270 | 19.378 | 5.587 | 15.687 | 1.00 | 0.79 | 1SG | 2219 |
| ATOM | 2219 | N | VAL | 271 | 17.739 | 5.855 | 17.235 | 1.00 | 0.48 | 1SG | 2220 |
| ATOM | 2220 | CA | VAL | 271 | 16.679 | 5.836 | 16.271 | 1.00 | 0.48 | 1SG | 2221 |
| ATOM | 2221 | CB | VAL | 271 | 16.262 | 4.467 | 15.804 | 1.00 | 0.48 | 1SG | 2222 |
| ATOM | 2222 | CG1 | VAL | 271 | 15.081 | 4.647 | 14.834 | 1.00 | 0.48 | 1SG | 2223 |
| ATOM | 2223 | CG2 | VAL | 271 | 17.452 | 3.716 | 15.186 | 1.00 | 0.48 | 1SG | 2224 |
| ATOM | 2224 | C | VAL | 271 | 15.456 | 6.387 | 16.934 | 1.00 | 0.48 | 1SG | 2225 |
| ATOM | 2225 | O | VAL | 271 | 14.809 | 5.685 | 17.709 | 1.00 | 0.48 | 1SG | 2226 |
| ATOM | 2226 | N | VAL | 272 | 15.105 | 7.663 | 16.677 | 1.00 | 0.62 | 1SG | 2227 |
| ATOM | 2227 | CA | VAL | 272 | 13.838 | 8.107 | 17.179 | 1.00 | 0.62 | 1SG | 2228 |
| ATOM | 2228 | CB | VAL | 272 | 13.810 | 9.562 | 17.542 | 1.00 | 0.62 | 1SG | 2229 |
| ATOM | 2229 | CG1 | VAL | 272 | 12.397 | 9.923 | 18.032 | 1.00 | 0.62 | 1SG | 2230 |
| ATOM | 2230 | CG2 | VAL | 272 | 14.914 | 9.829 | 18.577 | 1.00 | 0.62 | 1SG | 2231 |
| ATOM | 2231 | C | VAL | 272 | 12.979 | 7.933 | 15.988 | 1.00 | 0.62 | 1SG | 2232 |
| ATOM | 2232 | O | VAL | 272 | 12.368 | 8.878 | 15.493 | 1.00 | 0.62 | 1SG | 2233 |
| ATOM | 2233 | N | ARG | 273 | 12.888 | 6.684 | 15.504 | 1.00 | 0.88 | 1SG | 2234 |
| ATOM | 2234 | CA | ARG | 273 | 12.227 | 6.570 | 14.247 | 1.00 | 0.88 | 1SG | 2235 |
| ATOM | 2235 | CB | ARG | 273 | 13.111 | 6.986 | 13.058 | 1.00 | 0.88 | 1SG | 2236 |
| ATOM | 2236 | CG | ARG | 273 | 13.600 | 8.429 | 13.086 | 1.00 | 0.88 | 1SG | 2237 |
| ATOM | 2237 | CD | ARG | 273 | 14.474 | 8.803 | 11.887 | 1.00 | 0.88 | 1SG | 2238 |
| ATOM | 2238 | NE | ARG | 273 | 15.715 | 7.981 | 11.946 | 1.00 | 0.88 | 1SG | 2239 |
| ATOM | 2239 | CZ | ARG | 273 | 16.456 | 7.789 | 10.815 | 1.00 | 0.88 | 1SG | 2240 |
| ATOM | 2240 | NH1 | ARG | 273 | 16.046 | 8.335 | 9.633 | 1.00 | 0.88 | 1SG | 2241 |
| ATOM | 2241 | NH2 | ARG | 273 | 17.603 | 7.052 | 10.863 | 1.00 | 0.88 | 1SG | 2242 |
| ATOM | 2242 | C | ARG | 273 | 11.913 | 5.148 | 13.958 | 1.00 | 0.88 | 1SG | 2243 |
| ATOM | 2243 | O | ARG | 273 | 11.137 | 4.514 | 14.669 | 1.00 | 0.88 | 1SG | 2244 |
| ATOM | 2244 | N | ASP | 274 | 12.577 | 4.649 | 12.887 | 1.00 | 0.73 | 1SG | 2245 |
| ATOM | 2245 | CA | ASP | 274 | 12.405 | 3.383 | 12.230 | 1.00 | 0.73 | 1SG | 2246 |
| ATOM | 2246 | CB | ASP | 274 | 13.517 | 3.073 | 11.212 | 1.00 | 0.73 | 1SG | 2247 |
| ATOM | 2247 | CG | ASP | 274 | 13.352 | 4.013 | 10.027 | 1.00 | 0.73 | 1SG | 2248 |
| ATOM | 2248 | OD1 | ASP | 274 | 12.324 | 4.739 | 9.985 | 1.00 | 0.73 | 1SG | 2249 |
| ATOM | 2249 | OD2 | ASP | 274 | 14.250 | 4.011 | 9.144 | 1.00 | 0.73 | 1SG | 2250 |
| ATOM | 2250 | C | ASP | 274 | 12.401 | 2.286 | 13.226 | 1.00 | 0.73 | 1SG | 2251 |
| ATOM | 2251 | O | ASP | 274 | 11.684 | 1.300 | 13.056 | 1.00 | 0.73 | 1SG | 2252 |
| ATOM | 2252 | N | THR | 275 | 13.195 | 2.389 | 14.302 | 1.00 | 0.70 | 1SG | 2253 |
| ATOM | 2253 | CA | THR | 275 | 13.058 | 1.334 | 15.259 | 1.00 | 0.70 | 1SG | 2254 |
| ATOM | 2254 | CB | THR | 275 | 14.269 | 1.168 | 16.134 | 1.00 | 0.70 | 1SG | 2255 |
| ATOM | 2255 | OG1 | THR | 275 | 14.117 | 0.027 | 16.966 | 1.00 | 0.70 | 1SG | 2256 |
| ATOM | 2256 | CG2 | THR | 275 | 14.473 | 2.437 | 16.978 | 1.00 | 0.70 | 1SG | 2257 |
| ATOM | 2257 | C | THR | 275 | 11.867 | 1.663 | 16.117 | 1.00 | 0.70 | 1SG | 2258 |
| ATOM | 2258 | O | THR | 275 | 11.910 | 1.543 | 17.341 | 1.00 | 0.70 | 1SG | 2259 |
| ATOM | 2259 | N | LEU | 276 | 10.749 | 2.062 | 15.469 | 1.00 | 0.80 | 1SG | 2260 |
| ATOM | 2260 | CA | LEU | 276 | 9.533 | 2.423 | 16.128 | 1.00 | 0.80 | 1SG | 2261 |
| ATOM | 2261 | CB | LEU | 276 | 8.328 | 2.570 | 15.175 | 1.00 | 0.80 | 1SG | 2262 |
| ATOM | 2262 | CG | LEU | 276 | 8.469 | 3.510 | 13.964 | 1.00 | 0.80 | 1SG | 2263 |
| ATOM | 2263 | CD2 | LEU | 276 | 7.121 | 3.644 | 13.236 | 1.00 | 0.80 | 1SG | 2264 |
| ATOM | 2264 | CD1 | LEU | 276 | 9.556 | 3.025 | 12.996 | 1.00 | 0.80 | 1SG | 2265 |
| ATOM | 2265 | C | LEU | 276 | 9.174 | 1.199 | 16.869 | 1.00 | 0.80 | 1SG | 2266 |
| ATOM | 2266 | O | LEU | 276 | 9.527 | 0.988 | 18.027 | 1.00 | 0.80 | 1SG | 2267 |
| ATOM | 2267 | N | HIS | 277 | 8.436 | 0.354 | 16.141 | 1.00 | 0.77 | 1SG | 2268 |
| ATOM | 2268 | CA | HIS | 277 | 8.011 | −0.923 | 16.591 | 1.00 | 0.77 | 1SG | 2269 |
| ATOM | 2269 | ND1 | HIS | 277 | 5.323 | 0.169 | 18.180 | 1.00 | 0.77 | 1SG | 2270 |
| ATOM | 2270 | CG | HIS | 277 | 5.662 | −0.026 | 16.860 | 1.00 | 0.77 | 1SG | 2271 |
| ATOM | 2271 | CB | HIS | 277 | 6.517 | −1.147 | 16.346 | 1.00 | 0.77 | 1SG | 2272 |
| ATOM | 2272 | NE2 | HIS | 277 | 4.359 | 1.810 | 17.029 | 1.00 | 0.77 | 1SG | 2273 |
| ATOM | 2273 | CD2 | HIS | 277 | 5.065 | 0.986 | 16.171 | 1.00 | 0.77 | 1SG | 2274 |
| ATOM | 2274 | CE1 | HIS | 277 | 4.543 | 1.279 | 18.225 | 1.00 | 0.77 | 1SG | 2275 |
| ATOM | 2275 | C | HIS | 277 | 8.726 | −1.856 | 15.667 | 1.00 | 0.77 | 1SG | 2276 |
| ATOM | 2276 | O | HIS | 277 | 8.904 | −1.538 | 14.493 | 1.00 | 0.77 | 1SG | 2277 |
| ATOM | 2277 | N | LEU | 278 | 9.169 | −3.023 | 16.165 | 1.00 | 0.86 | 1SG | 2278 |
| ATOM | 2278 | CA | LEU | 278 | 9.903 | −3.917 | 15.319 | 1.00 | 0.86 | 1SG | 2279 |
| ATOM | 2279 | CB | LEU | 278 | 11.338 | −4.170 | 15.845 | 1.00 | 0.86 | 1SG | 2280 |
| ATOM | 2280 | CG | LEU | 278 | 12.273 | −5.088 | 15.017 | 1.00 | 0.86 | 1SG | 2281 |
| ATOM | 2281 | CD2 | LEU | 278 | 11.715 | −6.504 | 14.793 | 1.00 | 0.86 | 1SG | 2282 |
| ATOM | 2282 | CD1 | LEU | 278 | 13.661 | −5.156 | 15.675 | 1.00 | 0.86 | 1SG | 2283 |
| ATOM | 2283 | C | LEU | 278 | 9.140 | −5.195 | 15.313 | 1.00 | 0.86 | 1SG | 2284 |
| ATOM | 2284 | O | LEU | 278 | 8.610 | −5.617 | 16.339 | 1.00 | 0.86 | 1SG | 2285 |
| ATOM | 2285 | N | THR | 279 | 9.049 | −5.839 | 14.136 | 1.00 | 1.01 | 1SG | 2286 |
| ATOM | 2286 | CA | THR | 279 | 8.334 | −7.073 | 14.082 | 1.00 | 1.01 | 1SG | 2287 |
| ATOM | 2287 | CB | THR | 279 | 8.014 | −7.513 | 12.672 | 1.00 | 1.01 | 1SG | 2288 |
| ATOM | 2288 | OG1 | THR | 279 | 7.307 | −8.744 | 12.693 | 1.00 | 1.01 | 1SG | 2289 |
| ATOM | 2289 | CG2 | THR | 279 | 9.295 | −7.618 | 11.828 | 1.00 | 1.01 | 1SG | 2290 |
| ATOM | 2290 | C | THR | 279 | 9.161 | −8.101 | 14.784 | 1.00 | 1.01 | 1SG | 2291 |
| ATOM | 2291 | O | THR | 279 | 9.993 | −8.789 | 14.195 | 1.00 | 1.01 | 1SG | 2292 |
| ATOM | 2292 | N | ASN | 280 | 8.955 | −8.207 | 16.108 | 1.00 | 1.10 | 1SG | 2293 |
| ATOM | 2293 | CA | ASN | 280 | 9.691 | −9.162 | 16.877 | 1.00 | 1.10 | 1SG | 2294 |
| ATOM | 2294 | CB | ASN | 280 | 11.206 | −8.893 | 16.901 | 1.00 | 1.10 | 1SG | 2295 |
| ATOM | 2295 | CG | ASN | 280 | 11.431 | −7.553 | 17.587 | 1.00 | 1.10 | 1SG | 2296 |
| ATOM | 2296 | OD1 | ASN | 280 | 10.536 | −6.710 | 17.642 | 1.00 | 1.10 | 1SG | 2297 |
| ATOM | 2297 | ND2 | ASN | 280 | 12.664 | −7.347 | 18.121 | 1.00 | 1.10 | 1SG | 2298 |
| ATOM | 2298 | C | ASN | 280 | 9.202 | −9.043 | 18.279 | 1.00 | 1.10 | 1SG | 2299 |
| ATOM | 2299 | O | ASN | 280 | 8.137 | −8.484 | 18.534 | 1.00 | 1.10 | 1SG | 2300 |
| ATOM | 2300 | N | LYS | 281 | 9.978 | −9.590 | 19.232 | 1.00 | 1.14 | 1SG | 2301 |
| ATOM | 2301 | CA | LYS | 281 | 9.598 | −9.505 | 20.608 | 1.00 | 1.14 | 1SG | 2302 |
| ATOM | 2302 | CB | LYS | 281 | 10.005 | −10.752 | 21.413 | 1.00 | 1.14 | 1SG | 2303 |
| ATOM | 2303 | CG | LYS | 281 | 9.420 | −12.061 | 20.875 | 1.00 | 1.14 | 1SG | 2304 |
| ATOM | 2304 | CD | LYS | 281 | 7.896 | −12.159 | 20.952 | 1.00 | 1.14 | 1SG | 2305 |
| ATOM | 2305 | CE | LYS | 281 | 7.393 | −12.772 | 22.260 | 1.00 | 1.14 | 1SG | 2306 |
| ATOM | 2306 | NZ | LYS | 281 | 5.921 | −12.916 | 22.223 | 1.00 | 1.14 | 1SG | 2307 |
| ATOM | 2307 | C | LYS | 281 | 10.375 | −8.365 | 21.178 | 1.00 | 1.14 | 1SG | 2308 |
| ATOM | 2308 | O | LYS | 281 | 11.449 | −8.568 | 21.741 | 1.00 | 1.14 | 1SG | 2309 |
| ATOM | 2309 | N | LYS | 282 | 9.860 | −7.127 | 21.047 | 1.00 | 1.05 | 1SG | 2310 |
| ATOM | 2310 | CA | LYS | 282 | 10.609 | −6.029 | 21.580 | 1.00 | 1.05 | 1SG | 2311 |
| ATOM | 2311 | CB | LYS | 282 | 12.056 | −5.981 | 21.059 | 1.00 | 1.05 | 1SG | 2312 |
| ATOM | 2312 | CG | LYS | 282 | 12.969 | −5.052 | 21.862 | 1.00 | 1.05 | 1SG | 2313 |
| ATOM | 2313 | CD | LYS | 282 | 14.458 | −5.304 | 21.609 | 1.00 | 1.05 | 1SG | 2314 |
| ATOM | 2314 | CE | LYS | 282 | 14.951 | −6.643 | 22.163 | 1.00 | 1.05 | 1SG | 2315 |
| ATOM | 2315 | NZ | LYS | 282 | 16.359 | −6.870 | 21.767 | 1.00 | 1.05 | 1SG | 2316 |
| ATOM | 2316 | C | LYS | 282 | 9.928 | −4.758 | 21.182 | 1.00 | 1.05 | 1SG | 2317 |
| ATOM | 2317 | O | LYS | 282 | 8.952 | −4.340 | 21.803 | 1.00 | 1.05 | 1SG | 2318 |
| ATOM | 2318 | N | LYS | 283 | 10.431 | −4.114 | 20.111 | 1.00 | 0.77 | 1SG | 2319 |
| ATOM | 2319 | CA | LYS | 283 | 9.898 | −2.861 | 19.674 | 1.00 | 0.77 | 1SG | 2320 |
| ATOM | 2320 | CB | LYS | 283 | 10.625 | −2.279 | 18.450 | 1.00 | 0.77 | 1SG | 2321 |
| ATOM | 2321 | CG | LYS | 283 | 12.096 | −1.927 | 18.682 | 1.00 | 0.77 | 1SG | 2322 |
| ATOM | 2322 | CD | LYS | 283 | 13.012 | −3.146 | 18.805 | 1.00 | 0.77 | 1SG | 2323 |
| ATOM | 2323 | CE | LYS | 283 | 14.490 | −2.785 | 18.963 | 1.00 | 0.77 | 1SG | 2324 |
| ATOM | 2324 | NZ | LYS | 283 | 14.711 | −2.153 | 20.282 | 1.00 | 0.77 | 1SG | 2325 |
| ATOM | 2325 | C | LYS | 283 | 8.464 | −3.073 | 19.323 | 1.00 | 0.77 | 1SG | 2326 |
| ATOM | 2326 | O | LYS | 283 | 7.647 | −2.172 | 19.512 | 1.00 | 0.77 | 1SG | 2327 |
| ATOM | 2327 | N | ASP | 284 | 8.132 | −4.270 | 18.793 | 1.00 | 0.40 | 1SG | 2328 |
| ATOM | 2328 | CA | ASP | 284 | 6.782 | −4.614 | 18.442 | 1.00 | 0.40 | 1SG | 2329 |
| ATOM | 2329 | CB | ASP | 284 | 6.603 | −6.115 | 18.137 | 1.00 | 0.40 | 1SG | 2330 |
| ATOM | 2330 | CG | ASP | 284 | 5.189 | −6.387 | 17.633 | 1.00 | 0.40 | 1SG | 2331 |
| ATOM | 2331 | OD1 | ASP | 284 | 4.367 | −5.436 | 17.580 | 1.00 | 0.40 | 1SG | 2332 |
| ATOM | 2332 | OD2 | ASP | 284 | 4.912 | −7.568 | 17.294 | 1.00 | 0.40 | 1SG | 2333 |
| ATOM | 2333 | C | ASP | 284 | 5.928 | −4.288 | 19.620 | 1.00 | 0.40 | 1SG | 2334 |
| ATOM | 2334 | O | ASP | 284 | 6.242 | −4.636 | 20.757 | 1.00 | 0.40 | 1SG | 2335 |
| ATOM | 2335 | N | PRO | 285 | 4.870 | −3.577 | 19.377 | 1.00 | 0.24 | 1SG | 2336 |
| ATOM | 2336 | CA | PRO | 285 | 3.988 | −3.206 | 20.437 | 1.00 | 0.24 | 1SG | 2337 |
| ATOM | 2337 | CD | PRO | 285 | 4.740 | −2.716 | 18.219 | 1.00 | 0.24 | 1SG | 2338 |
| ATOM | 2338 | CB | PRO | 285 | 3.145 | −2.043 | 19.909 | 1.00 | 0.24 | 1SG | 2339 |
| ATOM | 2339 | CG | PRO | 285 | 3.348 | −2.087 | 18.382 | 1.00 | 0.24 | 1SG | 2340 |
| ATOM | 2340 | C | PRO | 285 | 3.205 | −4.409 | 20.792 | 1.00 | 0.24 | 1SG | 2341 |
| ATOM | 2341 | O | PRO | 285 | 2.817 | −5.143 | 19.887 | 1.00 | 0.24 | 1SG | 2342 |
| ATOM | 2342 | N | PRO | 286 | 2.972 | −4.645 | 22.041 | 1.00 | 0.32 | 1SG | 2343 |
| ATOM | 2343 | CA | PRO | 286 | 2.184 | −5.793 | 22.342 | 1.00 | 0.32 | 1SG | 2344 |
| ATOM | 2344 | CD | PRO | 286 | 3.998 | −4.427 | 23.045 | 1.00 | 0.32 | 1SG | 2345 |
| ATOM | 2345 | CB | PRO | 286 | 2.553 | −6.222 | 23.765 | 1.00 | 0.32 | 1SG | 2346 |
| ATOM | 2346 | CG | PRO | 286 | 3.419 | −5.074 | 24.311 | 1.00 | 0.32 | 1SG | 2347 |
| ATOM | 2347 | C | PRO | 286 | 0.761 | −5.430 | 22.139 | 1.00 | 0.32 | 1SG | 2348 |
| ATOM | 2348 | O | PRO | 286 | 0.425 | −4.249 | 22.229 | 1.00 | 0.32 | 1SG | 2349 |
| ATOM | 2349 | N | PRO | 287 | −0.053 | −6.385 | 21.825 | 1.00 | 0.51 | 1SG | 2350 |
| ATOM | 2350 | CA | PRO | 287 | −1.439 | −6.077 | 21.681 | 1.00 | 0.51 | 1SG | 2351 |
| ATOM | 2351 | CD | PRO | 287 | 0.354 | −7.480 | 20.963 | 1.00 | 0.51 | 1SG | 2352 |
| ATOM | 2352 | CB | PRO | 287 | −2.032 | −7.140 | 20.759 | 1.00 | 0.51 | 1SG | 2353 |
| ATOM | 2353 | CG | PRO | 287 | −0.955 | −8.237 | 20.691 | 1.00 | 0.51 | 1SG | 2354 |
| ATOM | 2354 | C | PRO | 287 | −2.038 | −6.066 | 23.035 | 1.00 | 0.51 | 1SG | 2355 |
| ATOM | 2355 | O | PRO | 287 | −1.706 | −6.932 | 23.841 | 1.00 | 0.51 | 1SG | 2356 |
| ATOM | 2356 | N | TYR | 288 | −2.915 | −5.102 | 23.328 | 1.00 | 0.64 | 1SG | 2357 |
| ATOM | 2357 | CA | TYR | 288 | −3.517 | −5.184 | 24.611 | 1.00 | 0.64 | 1SG | 2358 |
| ATOM | 2358 | CB | TYR | 288 | −2.985 | −4.172 | 25.642 | 1.00 | 0.64 | 1SG | 2359 |
| ATOM | 2359 | CG | TYR | 288 | −1.664 | −4.716 | 26.086 | 1.00 | 0.64 | 1SG | 2360 |
| ATOM | 2360 | CD1 | TYR | 288 | −0.503 | −4.445 | 25.395 | 1.00 | 0.64 | 1SG | 2361 |
| ATOM | 2361 | CD2 | TYR | 288 | −1.595 | −5.523 | 27.201 | 1.00 | 0.64 | 1SG | 2362 |
| ATOM | 2362 | CE1 | TYR | 288 | 0.702 | −4.963 | 25.813 | 1.00 | 0.64 | 1SG | 2363 |
| ATOM | 2363 | CE2 | TYR | 288 | −0.394 | −6.045 | 27.625 | 1.00 | 0.64 | 1SG | 2364 |
| ATOM | 2364 | CZ | TYR | 288 | 0.759 | −5.764 | 26.929 | 1.00 | 0.64 | 1SG | 2365 |
| ATOM | 2365 | OH | TYR | 288 | 1.997 | −6.294 | 27.355 | 1.00 | 0.64 | 1SG | 2366 |
| ATOM | 2366 | C | TYR | 288 | −4.978 | −5.048 | 24.425 | 1.00 | 0.64 | 1SG | 2367 |
| ATOM | 2367 | O | TYR | 288 | −5.461 | −4.044 | 23.903 | 1.00 | 0.64 | 1SG | 2368 |
| ATOM | 2368 | N | ASN | 289 | −5.712 | −6.101 | 24.833 | 1.00 | 0.62 | 1SG | 2369 |
| ATOM | 2369 | CA | ASN | 289 | −7.132 | −6.070 | 24.706 | 1.00 | 0.62 | 1SG | 2370 |
| ATOM | 2370 | CB | ASN | 289 | −7.838 | −7.266 | 25.376 | 1.00 | 0.62 | 1SG | 2371 |
| ATOM | 2371 | CG | ASN | 289 | −7.449 | −8.565 | 24.688 | 1.00 | 0.62 | 1SG | 2372 |
| ATOM | 2372 | OD1 | ASN | 289 | −7.100 | −9.540 | 25.352 | 1.00 | 0.62 | 1SG | 2373 |
| ATOM | 2373 | ND2 | ASN | 289 | −7.522 | −8.584 | 23.330 | 1.00 | 0.62 | 1SG | 2374 |
| ATOM | 2374 | C | ASN | 289 | −7.533 | −4.892 | 25.506 | 1.00 | 0.62 | 1SG | 2375 |
| ATOM | 2375 | O | ASN | 289 | −8.350 | −4.073 | 25.089 | 1.00 | 0.62 | 1SG | 2376 |
| ATOM | 2376 | N | LEU | 290 | −6.913 | −4.776 | 26.690 | 1.00 | 0.54 | 1SG | 2377 |
| ATOM | 2377 | CA | LEU | 290 | −7.241 | −3.710 | 27.570 | 1.00 | 0.54 | 1SG | 2378 |
| ATOM | 2378 | CB | LEU | 290 | −7.878 | −4.212 | 28.879 | 1.00 | 0.54 | 1SG | 2379 |
| ATOM | 2379 | CG | LEU | 290 | −9.296 | −4.780 | 28.653 | 1.00 | 0.54 | 1SG | 2380 |
| ATOM | 2380 | CD2 | LEU | 290 | −9.963 | −5.159 | 29.979 | 1.00 | 0.54 | 1SG | 2381 |
| ATOM | 2381 | CD1 | LEU | 290 | −9.302 | −5.940 | 27.647 | 1.00 | 0.54 | 1SG | 2382 |
| ATOM | 2382 | C | LEU | 290 | −5.979 | −2.971 | 27.852 | 1.00 | 0.54 | 1SG | 2383 |
| ATOM | 2383 | O | LEU | 290 | −4.879 | −3.513 | 27.747 | 1.00 | 0.54 | 1SG | 2384 |
| ATOM | 2384 | N | THR | 291 | −6.118 | −1.682 | 28.202 | 1.00 | 0.61 | 1SG | 2385 |
| ATOM | 2385 | CA | THR | 291 | −4.967 | −0.863 | 28.410 | 1.00 | 0.61 | 1SG | 2386 |
| ATOM | 2336 | CB | THR | 291 | −4.670 | 0.051 | 27.255 | 1.00 | 0.61 | 1SG | 2387 |
| ATOM | 2387 | OG1 | THR | 291 | −5.710 | 1.003 | 27.095 | 1.00 | 0.61 | 1SG | 2388 |
| ATOM | 2388 | CG2 | THR | 291 | −4.521 | −0.797 | 25.979 | 1.00 | 0.61 | 1SG | 2389 |
| ATOM | 2389 | C | THR | 291 | −5.225 | −0.005 | 29.605 | 1.00 | 0.61 | 1SG | 2390 |
| ATOM | 2390 | O | THR | 291 | −5.849 | −0.427 | 30.576 | 1.00 | 0.61 | 1SG | 2391 |
| ATOM | 2391 | N | MET | 292 | −4.710 | 1.237 | 29.555 | 1.00 | 0.96 | 1SG | 2392 |
| ATOM | 2392 | CA | MET | 292 | −4.819 | 2.157 | 30.649 | 1.00 | 0.96 | 1SG | 2393 |
| ATOM | 2393 | CB | MET | 292 | −4.172 | 3.517 | 30.329 | 1.00 | 0.96 | 1SG | 2394 |
| ATOM | 2394 | CG | MET | 292 | −4.271 | 4.527 | 31.474 | 1.00 | 0.96 | 1SG | 2395 |
| ATOM | 2395 | SD | MET | 292 | −3.528 | 6.146 | 31.122 | 1.00 | 0.96 | 1SG | 2396 |
| ATOM | 2396 | CE | MET | 292 | −1.844 | 5.681 | 31.604 | 1.00 | 0.96 | 1SG | 2397 |
| ATOM | 2397 | C | MET | 292 | −6.260 | 2.406 | 30.933 | 1.00 | 0.96 | 1SG | 2398 |
| ATOM | 2398 | O | MET | 292 | −6.697 | 2.381 | 32.083 | 1.00 | 0.96 | 1SG | 2399 |
| ATOM | 2399 | N | PHE | 293 | −7.052 | 2.658 | 29.880 | 1.00 | 1.23 | 1SG | 2400 |
| ATOM | 2400 | CA | PHE | 293 | −8.444 | 2.894 | 30.102 | 1.00 | 1.23 | 1SG | 2401 |
| ATOM | 2401 | CB | PHE | 293 | −9.120 | 1.794 | 30.960 | 1.00 | 1.23 | 1SG | 2402 |
| ATOM | 2402 | CG | PHE | 293 | −10.597 | 1.809 | 30.722 | 1.00 | 1.23 | 1SG | 2403 |
| ATOM | 2403 | CD1 | PHE | 293 | −11.124 | 1.173 | 29.621 | 1.00 | 1.23 | 1SG | 2404 |
| ATOM | 2404 | CD2 | PHE | 293 | −11.469 | 2.419 | 31.594 | 1.00 | 1.23 | 1SG | 2405 |
| ATOM | 2405 | CE1 | PHE | 293 | −12.478 | 1.171 | 29.377 | 1.00 | 1.23 | 1SG | 2406 |
| ATOM | 2406 | CE2 | PHE | 293 | −12.821 | 2.426 | 31.363 | 1.00 | 1.23 | 1SG | 2407 |
| ATOM | 2407 | CZ | PHE | 293 | −13.327 | 1.807 | 30.248 | 1.00 | 1.23 | 1SG | 2408 |
| ATOM | 2408 | C | PHE | 293 | −8.595 | 4.252 | 30.728 | 1.00 | 1.23 | 1SG | 2409 |
| ATOM | 2409 | O | PHE | 293 | −7.920 | 5.205 | 30.348 | 1.00 | 1.23 | 1SG | 2410 |
| ATOM | 2410 | N | THR | 294 | −9.478 | 4.379 | 31.733 | 1.00 | 1.14 | 1SG | 2411 |
| ATOM | 2411 | CA | THR | 294 | −9.806 | 5.647 | 32.317 | 1.00 | 1.14 | 1SG | 2412 |
| ATOM | 2412 | CB | THR | 294 | −11.272 | 5.754 | 32.608 | 1.00 | 1.14 | 1SG | 2413 |
| ATOM | 2413 | OG1 | THR | 294 | −11.671 | 4.752 | 33.530 | 1.00 | 1.14 | 1SG | 2414 |
| ATOM | 2414 | CG2 | THR | 294 | −12.017 | 5.555 | 31.278 | 1.00 | 1.14 | 1SG | 2415 |
| ATOM | 2415 | C | THR | 294 | −9.021 | 5.883 | 33.570 | 1.00 | 1.14 | 1SG | 2416 |
| ATOM | 2416 | O | THR | 294 | −8.014 | 5.230 | 33.829 | 1.00 | 1.14 | 1SG | 2417 |
| ATOM | 2417 | N | GLY | 295 | −9.446 | 6.875 | 34.380 | 1.00 | 0.80 | 1SG | 2418 |
| ATOM | 2418 | CA | GLY | 295 | −8.721 | 7.163 | 35.584 | 1.00 | 0.80 | 1SG | 2419 |
| ATOM | 2419 | C | GLY | 295 | −9.619 | 7.961 | 36.466 | 1.00 | 0.80 | 1SG | 2420 |
| ATOM | 2420 | O | GLY | 295 | −10.010 | 9.074 | 36.113 | 1.00 | 0.80 | 1SG | 2421 |
| ATOM | 2421 | N | ASN | 296 | −9.946 | 7.414 | 37.654 | 1.00 | 0.51 | 1SG | 2422 |
| ATOM | 2422 | CA | ASN | 296 | −10.857 | 8.058 | 38.557 | 1.00 | 0.51 | 1SG | 2423 |
| ATOM | 2423 | CB | ASN | 296 | −10.844 | 7.425 | 39.963 | 1.00 | 0.51 | 1SG | 2424 |
| ATOM | 2424 | CG | ASN | 296 | −11.168 | 5.939 | 39.891 | 1.00 | 0.51 | 1SG | 2425 |
| ATOM | 2425 | OD1 | ASN | 296 | −10.575 | 5.115 | 40.586 | 1.00 | 0.51 | 1SG | 2426 |
| ATOM | 2426 | ND2 | ASN | 296 | −12.150 | 5.576 | 39.027 | 1.00 | 0.51 | 1SG | 2427 |
| ATOM | 2427 | C | ASN | 296 | −10.334 | 9.431 | 38.810 | 1.00 | 0.51 | 1SG | 2428 |
| ATOM | 2428 | O | ASN | 296 | −10.990 | 10.437 | 38.559 | 1.00 | 0.51 | 1SG | 2429 |
| ATOM | 2429 | N | ALA | 297 | −9.126 | 9.482 | 39.370 | 1.00 | 0.52 | 1SG | 2430 |
| ATOM | 2430 | CA | ALA | 297 | −8.463 | 10.701 | 39.675 | 1.00 | 0.52 | 1SG | 2431 |
| ATOM | 2431 | CB | ALA | 297 | −7.554 | 10.524 | 40.874 | 1.00 | 0.52 | 1SG | 2432 |
| ATOM | 2432 | C | ALA | 297 | −7.705 | 11.222 | 38.489 | 1.00 | 0.52 | 1SG | 2433 |
| ATOM | 2433 | O | ALA | 297 | −7.389 | 12.402 | 38.427 | 1.00 | 0.52 | 1SG | 2434 |
| ATOM | 2434 | N | TYR | 298 | −7.417 | 10.350 | 37.504 | 1.00 | 0.62 | 1SG | 2435 |
| ATOM | 2435 | CA | TYR | 298 | −6.500 | 10.673 | 36.449 | 1.00 | 0.62 | 1SG | 2436 |
| ATOM | 2436 | CB | TYR | 298 | −6.462 | 9.645 | 35.317 | 1.00 | 0.62 | 1SG | 2437 |
| ATOM | 2437 | CG | TYR | 298 | −5.481 | 8.606 | 35.710 | 1.00 | 0.62 | 1SG | 2438 |
| ATOM | 2438 | CD1 | TYR | 298 | −4.159 | 8.815 | 35.400 | 1.00 | 0.62 | 1SG | 2439 |
| ATOM | 2439 | CD2 | TYR | 298 | −5.837 | 7.455 | 36.369 | 1.00 | 0.62 | 1SG | 2440 |
| ATOM | 2440 | CE1 | TYR | 298 | −3.193 | 7.905 | 35.736 | 1.00 | 0.62 | 1SG | 2441 |
| ATOM | 2441 | CE2 | TYR | 298 | −4.871 | 6.533 | 36.709 | 1.00 | 0.62 | 1SG | 2442 |
| ATOM | 2442 | CZ | TYR | 298 | −3.549 | 6.756 | 36.395 | 1.00 | 0.62 | 1SG | 2443 |
| ATOM | 2443 | OH | TYR | 298 | −2.559 | 5.816 | 36.744 | 1.00 | 0.62 | 1SG | 2444 |
| ATOM | 2444 | C | TYR | 298 | −6.697 | 11.982 | 35.768 | 1.00 | 0.62 | 1SG | 2445 |
| ATOM | 2445 | O | TYR | 298 | −5.826 | 12.834 | 35.893 | 1.00 | 0.62 | 1SG | 2446 |
| ATOM | 2446 | N | ILE | 299 | −7.804 | 12.249 | 35.051 | 1.00 | 0.59 | 1SG | 2447 |
| ATOM | 2447 | CA | ILE | 299 | −7.691 | 13.494 | 34.339 | 1.00 | 0.59 | 1SG | 2448 |
| ATOM | 2448 | CB | ILE | 299 | −7.564 | 13.336 | 32.856 | 1.00 | 0.59 | 1SG | 2449 |
| ATOM | 2449 | CG2 | ILE | 299 | −7.431 | 14.747 | 32.260 | 1.00 | 0.59 | 1SG | 2450 |
| ATOM | 2450 | CG1 | ILE | 299 | −6.368 | 12.428 | 32.518 | 1.00 | 0.59 | 1SG | 2451 |
| ATOM | 2451 | CD1 | ILE | 299 | −6.342 | 11.977 | 31.057 | 1.00 | 0.59 | 1SG | 2452 |
| ATOM | 2452 | C | ILE | 299 | −8.860 | 14.376 | 34.607 | 1.00 | 0.59 | 1SG | 2453 |
| ATOM | 2453 | O | ILE | 299 | −10.007 | 13.929 | 34.619 | 1.00 | 0.59 | 1SG | 2454 |
| ATOM | 2454 | N | VAL | 300 | −8.515 | 15.668 | 34.799 | 1.00 | 0.62 | 1SG | 2455 |
| ATOM | 2455 | CA | VAL | 300 | −9.281 | 16.836 | 35.132 | 1.00 | 0.62 | 1SG | 2456 |
| ATOM | 2456 | CB | VAL | 300 | −10.792 | 16.709 | 35.269 | 1.00 | 0.62 | 1SG | 2457 |
| ATOM | 2457 | CG1 | VAL | 300 | −11.254 | 15.665 | 36.307 | 1.00 | 0.62 | 1SG | 2458 |
| ATOM | 2458 | CG2 | VAL | 300 | −11.308 | 18.130 | 35.573 | 1.00 | 0.62 | 1SG | 2459 |
| ATOM | 2459 | C | VAL | 300 | −8.627 | 17.317 | 36.378 | 1.00 | 0.62 | 1SG | 2460 |
| ATOM | 2460 | O | VAL | 300 | −7.887 | 18.300 | 36.362 | 1.00 | 0. 62 | 1SG | 2461 |
| ATOM | 2461 | N | ALA | 301 | −8.912 | 16.639 | 37.500 | 1.00 | 0.68 | 1SG | 2462 |
| ATOM | 2462 | CA | ALA | 301 | −8.186 | 16.859 | 38.706 | 1.00 | 0.68 | 1SG | 2463 |
| ATOM | 2463 | CB | ALA | 301 | −9.091 | 17.116 | 39.923 | 1.00 | 0.68 | 1SG | 2464 |
| ATOM | 2464 | C | ALA | 301 | −7.475 | 15.557 | 38.913 | 1.00 | 0.68 | 1SG | 2465 |
| ATOM | 2465 | O | ALA | 301 | −8.006 | 14.645 | 39.547 | 1.00 | 0.68 | 1SG | 2466 |
| ATOM | 2466 | N | SER | 302 | −6.266 | 15.440 | 38.325 | 1.00 | 0.82 | 1SG | 2467 |
| ATOM | 2467 | CA | SER | 302 | −5.444 | 14.267 | 38.377 | 1.00 | 0.82 | 1SG | 2468 |
| ATOM | 2468 | CB | SER | 302 | −4.208 | 14.342 | 37.464 | 1.00 | 0.82 | 1SG | 2469 |
| ATOM | 2469 | OG | SER | 302 | −3.405 | 15.473 | 37.759 | 1.00 | 0.82 | 1SG | 2470 |
| ATOM | 2470 | C | SER | 302 | −4.961 | 14.103 | 39.771 | 1.00 | 0.82 | 1SG | 2471 |
| ATOM | 2471 | O | SER | 302 | −3.806 | 14.390 | 40.070 | 1.00 | 0.82 | 1SG | 2472 |
| ATOM | 2472 | N | ARG | 303 | −5.857 | 13.657 | 40.663 | 1.00 | 0.91 | 1SG | 2473 |
| ATOM | 2473 | CA | ARG | 303 | −5.588 | 13.475 | 42.057 | 1.00 | 0.91 | 1SG | 2474 |
| ATOM | 2474 | CB | ARG | 303 | −6.895 | 13.126 | 42.804 | 1.00 | 0.91 | 1SG | 2475 |
| ATOM | 2475 | CG | ARG | 303 | −6.843 | 13.254 | 44.326 | 1.00 | 0.91 | 1SG | 2476 |
| ATOM | 2476 | CD | ARG | 303 | −7.071 | 14.687 | 44.815 | 1.00 | 0.91 | 1SG | 2477 |
| ATOM | 2477 | NE | ARG | 303 | −8.464 | 15.087 | 44.447 | 1.00 | 0.91 | 1SG | 2478 |
| ATOM | 2478 | CZ | ARG | 303 | −9.520 | 14.692 | 45.217 | 1.00 | 0.91 | 1SG | 2479 |
| ATOM | 2479 | NH1 | ARG | 303 | −9.320 | 13.815 | 46.243 | 1.00 | 0.91 | 1SG | 2480 |
| ATOM | 2480 | NH2 | ARG | 303 | −10.776 | 15.159 | 44.952 | 1.00 | 0.91 | 1SG | 2481 |
| ATOM | 2481 | C | ARG | 303 | −4.633 | 12.337 | 42.287 | 1.00 | 0.91 | 1SG | 2482 |
| ATOM | 2482 | O | ARG | 303 | −3.598 | 12.471 | 42.936 | 1.00 | 0.91 | 1SG | 2483 |
| ATOM | 2483 | N | ASP | 304 | −4.986 | 11.176 | 41.726 | 1.00 | 0.60 | 1SG | 2484 |
| ATOM | 2484 | CA | ASP | 304 | −4.390 | 9.897 | 41.955 | 1.00 | 0.60 | 1SG | 2485 |
| ATOM | 2485 | CB | ASP | 304 | −5.249 | 8.764 | 41.357 | 1.00 | 0.60 | 1SG | 2486 |
| ATOM | 2486 | CG | ASP | 304 | −4.826 | 7.453 | 41.978 | 1.00 | 0.60 | 1SG | 2487 |
| ATOM | 2487 | OD1 | ASP | 304 | −3.952 | 7.486 | 42.883 | 1.00 | 0.60 | 1SG | 2488 |
| ATOM | 2488 | OD2 | ASP | 304 | −5.373 | 6.398 | 41.560 | 1.00 | 0.60 | 1SG | 2489 |
| ATOM | 2489 | C | ASP | 304 | −3.027 | 9.767 | 41.382 | 1.00 | 0.60 | 1SG | 2490 |
| ATOM | 2490 | O | ASP | 304 | −2.081 | 9.450 | 42.094 | 1.00 | 0.60 | 1SG | 2491 |
| ATOM | 2491 | N | PHE | 305 | −2.854 | 10.031 | 40.079 | 1.00 | 0.35 | 1SG | 2492 |
| ATOM | 2492 | CA | PHE | 305 | −1.546 | 9.740 | 39.583 | 1.00 | 0.35 | 1SG | 2493 |
| ATOM | 2493 | CB | PHE | 305 | −1.418 | 9.688 | 38.058 | 1.00 | 0.35 | 1SG | 2494 |
| ATOM | 2494 | CG | PHE | 305 | −0.157 | 8.915 | 37.889 | 1.00 | 0.35 | 1SG | 2495 |
| ATOM | 2495 | CD1 | PHE | 305 | −0.191 | 7.538 | 37.943 | 1.00 | 0.35 | 1SG | 2496 |
| ATOM | 2496 | CD2 | PHE | 305 | 1.050 | 9.548 | 37.705 | 1.00 | 0.35 | 1SG | 2497 |
| ATOM | 2497 | CE1 | PHE | 305 | 0.959 | 6.799 | 37.807 | 1.00 | 0.35 | 1SG | 2498 |
| ATOM | 2498 | CE2 | PHE | 305 | 2.204 | 8.814 | 37.566 | 1.00 | 0.35 | 1SG | 2499 |
| ATOM | 2499 | CZ | PHE | 305 | 2.159 | 7.441 | 37.618 | 1.00 | 0.35 | 1SG | 2500 |
| ATOM | 2500 | C | PHE | 305 | −0.578 | 10.737 | 40.115 | 1.00 | 0.35 | 1SG | 2501 |
| ATOM | 2501 | O | PHE | 305 | 0.569 | 10.403 | 40.407 | 1.00 | 0.35 | 1SG | 2502 |
| ATOM | 2502 | N | VAL | 306 | −1.025 | 11.994 | 40.275 | 1.00 | 0.40 | 1SG | 2503 |
| ATOM | 2503 | CA | VAL | 306 | −0.112 | 13.002 | 40.711 | 1.00 | 0.40 | 1SG | 2504 |
| ATOM | 2504 | CB | VAL | 306 | −0.712 | 14.368 | 40.868 | 1.00 | 0.40 | 1SG | 2505 |
| ATOM | 2505 | CG1 | VAL | 306 | −1.176 | 14.883 | 39.495 | 1.00 | 0.40 | 1SG | 2506 |
| ATOM | 2506 | CG2 | VAL | 306 | −1.805 | 14.303 | 41.950 | 1.00 | 0.40 | 1SG | 2507 |
| ATOM | 2507 | C | VAL | 306 | 0.423 | 12.616 | 42.040 | 1.00 | 0.40 | 1SG | 2508 |
| ATOM | 2508 | O | VAL | 306 | 1.576 | 12.914 | 42.346 | 1.00 | 0.40 | 1SG | 2509 |
| ATOM | 2509 | N | GLN | 307 | −0.395 | 11.943 | 42.867 | 1.00 | 0.44 | 1SG | 2510 |
| ATOM | 2510 | CA | GLN | 307 | 0.088 | 11.616 | 44.176 | 1.00 | 0.44 | 1SG | 2511 |
| ATOM | 2511 | CB | GLN | 307 | −0.877 | 10.791 | 45.050 | 1.00 | 0.44 | 1SG | 2512 |
| ATOM | 2512 | CG | GLN | 307 | −1.008 | 9.334 | 44.600 | 1.00 | 0.44 | 1SG | 2513 |
| ATOM | 2513 | CD | GLN | 307 | −2.029 | 8.638 | 45.483 | 1.00 | 0.44 | 1SG | 2514 |
| ATOM | 2514 | OE1 | GLN | 307 | −2.677 | 9.267 | 46.317 | 1.00 | 0.44 | 1SG | 2515 |
| ATOM | 2515 | NE2 | GLN | 307 | −2.178 | 7.299 | 45.297 | 1.00 | 0.44 | 1SG | 2516 |
| ATOM | 2516 | C | GLN | 307 | 1.297 | 10.761 | 44.003 | 1.00 | 0.44 | 1SG | 2517 |
| ATOM | 2517 | O | GLN | 307 | 2.265 | 10.899 | 44.743 | 1.00 | 0.44 | 1SG | 2518 |
| ATOM | 2518 | N | HIS | 308 | 1.283 | 9.863 | 43.000 | 1.00 | 0.44 | 1SG | 2519 |
| ATOM | 2519 | CA | HIS | 308 | 2.401 | 8.977 | 42.837 | 1.00 | 0.44 | 1SG | 2520 |
| ATOM | 2520 | ND1 | HIS | 308 | 4.581 | 7.430 | 40.882 | 1.00 | 0.44 | 1SG | 2521 |
| ATOM | 2521 | CG | HIS | 308 | 3.395 | 7.079 | 41.485 | 1.00 | 0.44 | 1SG | 2522 |
| ATOM | 2522 | CB | HIS | 308 | 2.232 | 8.008 | 41.653 | 1.00 | 0.44 | 1SG | 2523 |
| ATOM | 2523 | NE2 | HIS | 308 | 4.788 | 5.307 | 41.504 | 1.00 | 0.44 | 1SG | 2524 |
| ATOM | 2524 | CD2 | HIS | 308 | 3.539 | 5.780 | 41.859 | 1.00 | 0.44 | 1SG | 2525 |
| ATOM | 2525 | CE1 | HIS | 308 | 5.378 | 6.334 | 40.920 | 1.00 | 0.44 | 1SG | 2526 |
| ATOM | 2526 | C | HIS | 308 | 3.651 | 9.765 | 42.581 | 1.00 | 0.44 | 1SG | 2527 |
| ATOM | 2527 | O | HIS | 308 | 4.674 | 9.550 | 43.232 | 1.00 | 0.44 | 1SG | 2528 |
| ATOM | 2528 | N | VAL | 309 | 3.603 | 10.716 | 41.631 | 1.00 | 0.46 | 1SG | 2529 |
| ATOM | 2529 | CA | VAL | 309 | 4.798 | 11.447 | 41.311 | 1.00 | 0.46 | 1SG | 2530 |
| ATOM | 2530 | CB | VAL | 309 | 4.643 | 12.328 | 40.107 | 1.00 | 0.46 | 1SG | 2531 |
| ATOM | 2531 | CG1 | VAL | 309 | 4.580 | 11.422 | 38.868 | 1.00 | 0.46 | 1SG | 2532 |
| ATOM | 2532 | CG2 | VAL | 309 | 3.372 | 13.175 | 40.282 | 1.00 | 0.46 | 1SG | 2533 |
| ATOM | 2533 | C | VAL | 309 | 5.250 | 12.267 | 42.480 | 1.00 | 0.46 | 1SG | 2534 |
| ATOM | 2534 | O | VAL | 309 | 6.438 | 12.298 | 42.794 | 1.00 | 0.46 | 1SG | 2535 |
| ATOM | 2535 | N | LEU | 310 | 4.323 | 12.552 | 43.170 | 1.00 | 0.61 | 1SG | 2536 |
| ATOM | 2536 | CA | LEU | 310 | 4.718 | 13.752 | 44.294 | 1.00 | 0.61 | 1SG | 2537 |
| ATOM | 2537 | CB | LEU | 310 | 3.575 | 14.599 | 44.892 | 1.00 | 0.61 | 1SG | 2538 |
| ATOM | 2538 | CG | LEU | 310 | 3.152 | 15.826 | 44.055 | 1.00 | 0.61 | 1SG | 2539 |
| ATOM | 2539 | CD2 | LEU | 310 | 2.616 | 15.432 | 42.672 | 1.00 | 0.61 | 1SG | 2540 |
| ATOM | 2540 | CD1 | LEU | 310 | 4.285 | 16.860 | 43.979 | 1.00 | 0.61 | 1SG | 2541 |
| ATOM | 2541 | C | LEU | 310 | 5.228 | 12.865 | 45.388 | 1.00 | 0.61 | 1SG | 2542 |
| ATOM | 2542 | O | LEU | 310 | 6.165 | 13.225 | 46.098 | 1.00 | 0.61 | 1SG | 2543 |
| ATOM | 2543 | N | LYS | 311 | 4.621 | 11.676 | 45.544 | 1.00 | 0.78 | 1SG | 2544 |
| ATOM | 2544 | CA | LYS | 311 | 4.915 | 10.793 | 46.638 | 1.00 | 0.78 | 1SG | 2545 |
| ATOM | 2545 | CB | LYS | 311 | 4.060 | 9.507 | 46.614 | 1.00 | 0.78 | 1SG | 2546 |
| ATOM | 2546 | CG | LYS | 311 | 4.129 | 8.657 | 47.891 | 1.00 | 0.78 | 1SG | 2547 |
| ATOM | 2547 | CD | LYS | 311 | 5.501 | 8.045 | 48.189 | 1.00 | 0.78 | 1SG | 2548 |
| ATOM | 2548 | CE | LYS | 311 | 5.803 | 6.780 | 47.380 | 1.00 | 0.78 | 1SG | 2549 |
| ATOM | 2549 | NZ | LYS | 311 | 7.193 | 6.332 | 47.629 | 1.00 | 0.78 | 1SG | 2550 |
| ATOM | 2550 | C | LYS | 311 | 6.352 | 10.386 | 46.619 | 1.00 | 0.78 | 1SG | 2551 |
| ATOM | 2551 | O | LYS | 311 | 7.013 | 10.445 | 47.654 | 1.00 | 0.78 | 1SG | 2552 |
| ATOM | 2552 | N | ASN | 312 | 6.888 | 9.973 | 45.453 | 1.00 | 0.63 | 1SG | 2553 |
| ATOM | 2553 | CA | ASN | 312 | 8.258 | 9.546 | 45.441 | 1.00 | 0.63 | 1SG | 2554 |
| ATOM | 2554 | CB | ASN | 312 | 8.718 | 9.006 | 44.079 | 1.00 | 0.63 | 1SG | 2555 |
| ATOM | 2555 | CG | ASN | 312 | 8.433 | 10.068 | 43.047 | 1.00 | 0.63 | 1SG | 2556 |
| ATOM | 2556 | OD1 | ASN | 312 | 9.003 | 11.160 | 43.064 | 1.00 | 0.63 | 1SG | 2557 |
| ATOM | 2557 | ND2 | ASN | 312 | 7.489 | 9.718 | 42.132 | 1.00 | 0.63 | 1SG | 2558 |
| ATOM | 2558 | C | ASN | 312 | 9.099 | 10.693 | 45.902 | 1.00 | 0.63 | 1SG | 2559 |
| ATOM | 2559 | O | ASN | 312 | 8.706 | 11.848 | 45.747 | 1.00 | 0.63 | 1SG | 2560 |
| ATOM | 2560 | N | PRO | 313 | 10.238 | 10.378 | 46.482 | 1.00 | 0.40 | 1SG | 2561 |
| ATOM | 2561 | CA | PRO | 313 | 11.051 | 11.351 | 47.164 | 1.00 | 0.40 | 1SG | 2562 |
| ATOM | 2562 | CD | PRO | 313 | 11.042 | 9.269 | 45.994 | 1.00 | 0.40 | 1SG | 2563 |
| ATOM | 2563 | CB | PRO | 313 | 12.403 | 10.682 | 47.402 | 1.00 | 0.40 | 1SG | 2564 |
| ATOM | 2564 | CG | PRO | 313 | 12.504 | 9.674 | 46.247 | 1.00 | 0.40 | 1SG | 2565 |
| ATOM | 2565 | C | PRO | 313 | 11.199 | 12.657 | 46.470 | 1.00 | 0.40 | 1SG | 2566 |
| ATOM | 2566 | O | PRO | 313 | 11.990 | 12.767 | 45.532 | 1.00 | 0.40 | 1SG | 2567 |
| ATOM | 2567 | N | LYS | 314 | 10.424 | 13.650 | 46.946 | 1.00 | 0.42 | 1SG | 2568 |
| ATOM | 2568 | CA | LYS | 314 | 10.494 | 14.989 | 46.460 | 1.00 | 0.42 | 1SG | 2569 |
| ATOM | 2569 | CB | LYS | 314 | 9.283 | 15.833 | 46.890 | 1.00 | 0.42 | 1SG | 2570 |
| ATOM | 2570 | CG | LYS | 314 | 9.078 | 15.850 | 48.402 | 1.00 | 0.42 | 1SG | 2571 |
| ATOM | 2571 | CD | LYS | 314 | 8.025 | 16.853 | 48.874 | 1.00 | 0.42 | 1SG | 2572 |
| ATOM | 2572 | CE | LYS | 314 | 8.511 | 18.304 | 48.896 | 1.00 | 0.42 | 1SG | 2573 |
| ATOM | 2573 | NZ | LYS | 314 | 7.428 | 19.201 | 49.361 | 1.00 | 0.42 | 1SG | 2574 |
| ATOM | 2574 | C | LYS | 314 | 11.707 | 15.570 | 47.079 | 1.00 | 0.42 | 1SG | 2575 |
| ATOM | 2575 | O | LYS | 314 | 12.526 | 16.195 | 46.412 | 1.00 | 0.42 | 1SG | 2576 |
| ATOM | 2576 | N | SER | 315 | 11.870 | 15.316 | 48.388 | 1.00 | 0.52 | 1SG | 2577 |
| ATOM | 2577 | CA | SER | 315 | 12.975 | 15.842 | 49.123 | 1.00 | 0.52 | 1SG | 2578 |
| ATOM | 2578 | CB | SER | 315 | 12.976 | 15.400 | 50.596 | 1.00 | 0.52 | 1SG | 2579 |
| ATOM | 2579 | OG | SER | 315 | 14.094 | 15.962 | 51.267 | 1.00 | 0.52 | 1SG | 2580 |
| ATOM | 2580 | C | SER | 315 | 14.210 | 15.315 | 48.488 | 1.00 | 0.52 | 1SG | 2581 |
| ATOM | 2581 | O | SER | 315 | 15.191 | 16.037 | 48.314 | 1.00 | 0.52 | 1SG | 2582 |
| ATOM | 2582 | N | GLN | 316 | 14.198 | 14.023 | 48.117 | 1.00 | 0.66 | 1SG | 2583 |
| ATOM | 2583 | CA | GLN | 316 | 15.357 | 13.479 | 47.487 | 1.00 | 0.66 | 1SG | 2584 |
| ATOM | 2584 | CB | GLN | 316 | 15.510 | 11.958 | 47.670 | 1.00 | 0.66 | 1SG | 2585 |
| ATOM | 2585 | CG | GLN | 316 | 16.757 | 11.398 | 46.985 | 1.00 | 0.66 | 1SG | 2586 |
| ATOM | 2586 | CD | GLN | 316 | 16.799 | 9.897 | 47.232 | 1.00 | 0.66 | 1SG | 2587 |
| ATOM | 2587 | OE1 | GLN | 316 | 17.748 | 9.216 | 46.846 | 1.00 | 0.66 | 1SG | 2588 |
| ATOM | 2588 | NE2 | GLN | 316 | 15.743 | 9.366 | 47.906 | 1.00 | 0.66 | 1SG | 2589 |
| ATOM | 2589 | C | GLN | 316 | 15.218 | 13.728 | 46.020 | 1.00 | 0.66 | 1SG | 2590 |
| ATOM | 2590 | O | GLN | 316 | 15.165 | 12.789 | 45.226 | 1.00 | 0.66 | 1SG | 2591 |
| ATOM | 2591 | N | GLN | 317 | 15.145 | 15.010 | 45.617 | 1.00 | 0.78 | 1SG | 2592 |
| ATOM | 2592 | CA | GLN | 317 | 15.076 | 15.297 | 44.215 | 1.00 | 0.78 | 1SG | 2593 |
| ATOM | 2593 | CB | GLN | 317 | 14.254 | 16.556 | 43.857 | 1.00 | 0.78 | 1SG | 2594 |
| ATOM | 2594 | CG | GLN | 317 | 14.872 | 17.891 | 44.288 | 1.00 | 0.78 | 1SG | 2595 |
| ATOM | 2595 | CD | GLN | 317 | 14.774 | 18.068 | 45.795 | 1.00 | 0.78 | 1SG | 2596 |
| ATOM | 2596 | OE1 | GLN | 317 | 13.722 | 18.415 | 46.330 | 1.00 | 0.78 | 1SG | 2597 |
| ATOM | 2597 | NE2 | GLN | 317 | 15.915 | 17.839 | 46.499 | 1.00 | 0.78 | 1SG | 2598 |
| ATOM | 2598 | C | GLN | 317 | 16.489 | 15.565 | 43.805 | 1.00 | 0.78 | 1SG | 2599 |
| ATOM | 2599 | O | GLN | 317 | 17.167 | 16.394 | 44.408 | 1.00 | 0.78 | 1SG | 2600 |
| ATOM | 2600 | N | LEU | 318 | 17.001 | 14.835 | 42.794 | 1.00 | 0.79 | 1SG | 2601 |
| ATOM | 2601 | CA | LEU | 318 | 18.359 | 15.086 | 42.401 | 1.00 | 0.79 | 1SG | 2602 |
| ATOM | 2602 | CB | LEU | 318 | 19.298 | 13.896 | 42.669 | 1.00 | 0.79 | 1SG | 2603 |
| ATOM | 2603 | CG | LEU | 318 | 19.461 | 13.558 | 44.163 | 1.00 | 0.79 | 1SG | 2604 |
| ATOM | 2604 | CD2 | LEU | 318 | 20.539 | 12.482 | 44.376 | 1.00 | 0.79 | 1SG | 2605 |
| ATOM | 2605 | CD1 | LEU | 318 | 18.115 | 13.173 | 44.798 | 1.00 | 0.79 | 1SG | 2606 |
| ATOM | 2606 | C | LEU | 318 | 18.388 | 15.334 | 40.925 | 1.00 | 0.79 | 1SG | 2607 |
| ATOM | 2607 | O | LEU | 318 | 18.222 | 14.407 | 40.135 | 1.00 | 0.79 | 1SG | 2608 |
| ATOM | 2608 | N | ILE | 319 | 18.560 | 16.606 | 40.512 | 1.00 | 0.72 | 1SG | 2609 |
| ATOM | 2609 | CA | ILE | 319 | 18.642 | 16.912 | 39.113 | 1.00 | 0.72 | 1SG | 2610 |
| ATOM | 2610 | CB | ILE | 319 | 18.402 | 18.384 | 38.841 | 1.00 | 0.72 | 1SG | 2611 |
| ATOM | 2611 | CG2 | ILE | 319 | 19.513 | 19.211 | 39.512 | 1.00 | 0.72 | 1SG | 2612 |
| ATOM | 2612 | CG1 | ILE | 319 | 18.203 | 18.676 | 37.340 | 1.00 | 0.72 | 1SG | 2613 |
| ATOM | 2613 | CD1 | ILE | 319 | 19.434 | 18.463 | 36.459 | 1.00 | 0.72 | 1SG | 2614 |
| ATOM | 2614 | C | ILE | 319 | 19.976 | 16.486 | 38.565 | 1.00 | 0.72 | 1SG | 2615 |
| ATOM | 2615 | O | ILE | 319 | 20.051 | 15.789 | 37.553 | 1.00 | 0.72 | 1SG | 2616 |
| ATOM | 2616 | N | GLU | 320 | 21.069 | 16.881 | 39.248 | 1.00 | 0.46 | 1SG | 2617 |
| ATOM | 2617 | CA | GLU | 320 | 22.407 | 16.647 | 38.784 | 1.00 | 0.46 | 1SG | 2618 |
| ATOM | 2618 | CB | GLU | 320 | 23.465 | 17.381 | 39.628 | 1.00 | 0.46 | 1SG | 2619 |
| ATOM | 2619 | CG | GLU | 320 | 24.898 | 17.170 | 39.133 | 1.00 | 0.46 | 1SG | 2620 |
| ATOM | 2620 | CD | GLU | 320 | 25.073 | 17.950 | 37.836 | 1.00 | 0.46 | 1SG | 2621 |
| ATOM | 2621 | OE1 | GLU | 320 | 24.343 | 18.959 | 37.647 | 1.00 | 0.46 | 1SG | 2622 |
| ATOM | 2622 | OE2 | GLU | 320 | 25.940 | 17.545 | 37.017 | 1.00 | 0.46 | 1SG | 2623 |
| ATOM | 2623 | C | GLU | 320 | 22.728 | 15.194 | 38.829 | 1.00 | 0.46 | 1SG | 2624 |
| ATOM | 2624 | O | GLU | 320 | 23.342 | 14.657 | 37.909 | 1.00 | 0.46 | 1SG | 2625 |
| ATOM | 2625 | N | TRP | 321 | 22.303 | 14.511 | 39.903 | 1.00 | 0.19 | 1SG | 2626 |
| ATOM | 2626 | CA | TRP | 321 | 22.658 | 13.137 | 40.092 | 1.00 | 0.19 | 1SG | 2627 |
| ATOM | 2627 | CB | TRP | 321 | 22.123 | 12.576 | 41.417 | 1.00 | 0.19 | 1SG | 2628 |
| ATOM | 2628 | CG | TRP | 321 | 22.816 | 13.158 | 42.626 | 1.00 | 0.19 | 1SG | 2629 |
| ATOM | 2629 | CD2 | TRP | 321 | 23.907 | 12.520 | 43.306 | 1.00 | 0.19 | 1SG | 2630 |
| ATOM | 2630 | CD1 | TRP | 321 | 22.577 | 14.326 | 43.290 | 1.00 | 0.19 | 1SG | 2631 |
| ATOM | 2631 | NE1 | TRP | 321 | 23.454 | 14.457 | 44.341 | 1.00 | 0.19 | 1SG | 2632 |
| ATOM | 2632 | CE2 | TRP | 321 | 24.279 | 13.351 | 44.362 | 1.00 | 0.19 | 1SG | 2633 |
| ATOM | 2633 | CE3 | TRP | 321 | 24.547 | 11.337 | 43.069 | 1.00 | 0.19 | 1SG | 2634 |
| ATOM | 2634 | CZ2 | TRP | 321 | 25.302 | 13.011 | 45.200 | 1.00 | 0.19 | 1SG | 2635 |
| ATOM | 2635 | CZ3 | TRP | 321 | 25.578 | 10.997 | 43.917 | 1.00 | 0.19 | 1SG | 2636 |
| ATOM | 2636 | CH2 | TRP | 321 | 25.948 | 11.818 | 44.961 | 1.00 | 0.19 | 1SG | 2637 |
| ATOM | 2637 | C | TRP | 321 | 22.110 | 12.315 | 38.971 | 1.00 | 0.19 | 1SG | 2638 |
| ATOM | 2638 | O | TRP | 321 | 22.782 | 11.413 | 38.473 | 1.00 | 0.19 | 1SG | 2639 |
| ATOM | 2639 | N | VAL | 322 | 20.879 | 12.614 | 38.524 | 1.00 | 0.15 | 1SG | 2640 |
| ATOM | 2640 | CA | VAL | 322 | 20.300 | 11.804 | 37.497 | 1.00 | 0.15 | 1SG | 2641 |
| ATOM | 2641 | CB | VAL | 322 | 18.923 | 12.263 | 37.119 | 1.00 | 0.15 | 1SG | 2642 |
| ATOM | 2642 | CG1 | VAL | 322 | 18.385 | 11.348 | 36.009 | 1.00 | 0.15 | 1SG | 2643 |
| ATOM | 2643 | CG2 | VAL | 322 | 18.066 | 12.269 | 38.390 | 1.00 | 0.15 | 1SG | 2644 |
| ATOM | 2644 | C | VAL | 322 | 21.177 | 11.893 | 36.282 | 1.00 | 0.15 | 1SG | 2645 |
| ATOM | 2645 | O | VAL | 322 | 21.583 | 10.870 | 35.732 | 1.00 | 0.15 | 1SG | 2646 |
| ATOM | 2646 | N | LYS | 323 | 21.525 | 13.120 | 35.852 | 1.00 | 0.33 | 1SG | 2647 |
| ATOM | 2647 | CA | LYS | 323 | 22.341 | 13.253 | 34.680 | 1.00 | 0.33 | 1SG | 2648 |
| ATOM | 2648 | CB | LYS | 323 | 22.566 | 14.704 | 34.226 | 1.00 | 0.33 | 1SG | 2649 |
| ATOM | 2649 | CG | LYS | 323 | 23.440 | 14.775 | 32.972 | 1.00 | 0.33 | 1SG | 2650 |
| ATOM | 2650 | CD | LYS | 323 | 23.460 | 16.135 | 32.275 | 1.00 | 0.33 | 1SG | 2651 |
| ATOM | 2651 | CE | LYS | 323 | 24.447 | 16.198 | 31.107 | 1.00 | 0.33 | 1SG | 2652 |
| ATOM | 2652 | NZ | LYS | 323 | 24.407 | 17.534 | 30.474 | 1.00 | 0.33 | 1SG | 2653 |
| ATOM | 2653 | C | LYS | 323 | 23.687 | 12.677 | 34.947 | 1.00 | 0.33 | 1SG | 2654 |
| ATOM | 2654 | O | LYS | 323 | 24.234 | 11.967 | 34.106 | 1.00 | 0.33 | 1SG | 2655 |
| ATOM | 2655 | N | ASP | 324 | 24.256 | 12.964 | 36.132 | 1.00 | 0.49 | 1SG | 2656 |
| ATOM | 2656 | CA | ASP | 324 | 25.578 | 12.497 | 36.426 | 1.00 | 0.49 | 1SG | 2657 |
| ATOM | 2657 | CB | ASP | 324 | 26.214 | 13.183 | 37.650 | 1.00 | 0.49 | 1SG | 2658 |
| ATOM | 2658 | CG | ASP | 324 | 27.696 | 12.839 | 37.669 | 1.00 | 0.49 | 1SG | 2659 |
| ATOM | 2659 | OD1 | ASP | 324 | 28.114 | 11.983 | 36.844 | 1.00 | 0.49 | 1SG | 2660 |
| ATOM | 2660 | OD2 | ASP | 324 | 28.430 | 13.427 | 38.508 | 1.00 | 0.49 | 1SG | 2661 |
| ATOM | 2661 | C | ASP | 324 | 25.493 | 11.035 | 36.693 | 1.00 | 0.49 | 1SG | 2662 |
| ATOM | 2662 | O | ASP | 324 | 25.405 | 10.596 | 37.838 | 1.00 | 0.49 | 1SG | 2663 |
| ATOM | 2663 | N | THR | 325 | 25.505 | 10.243 | 35.609 | 1.00 | 0.73 | 1SG | 2664 |
| ATOM | 2664 | CA | THR | 325 | 25.434 | 8.821 | 35.700 | 1.00 | 0.73 | 1SG | 2665 |
| ATOM | 2665 | CB | THR | 325 | 25.467 | 8.173 | 34.349 | 1.00 | 0.73 | 1SG | 2666 |
| ATOM | 2666 | OG1 | THR | 325 | 24.325 | 8.536 | 33.586 | 1.00 | 0.73 | 1SG | 2667 |
| ATOM | 2667 | CG2 | THR | 325 | 25.565 | 6.652 | 34.529 | 1.00 | 0.73 | 1SG | 2668 |
| ATOM | 2668 | C | THR | 325 | 26.643 | 8.340 | 36.428 | 1.00 | 0.73 | 1SG | 2669 |
| ATOM | 2669 | O | THR | 325 | 26.538 | 7.553 | 37.369 | 1.00 | 0.73 | 1SG | 2670 |
| ATOM | 2670 | N | TYR | 326 | 27.830 | 8.825 | 36.018 | 1.00 | 1.11 | 1SG | 2671 |
| ATOM | 2671 | CA | TYR | 326 | 29.033 | 8.338 | 36.621 | 1.00 | 1.11 | 1SG | 2672 |
| ATOM | 2672 | CB | TYR | 326 | 30.319 | 8.825 | 35.933 | 1.00 | 1.11 | 1SG | 2673 |
| ATOM | 2673 | CG | TYR | 326 | 31.459 | 8.163 | 36.630 | 1.00 | 1.11 | 1SG | 2674 |
| ATOM | 2674 | CD1 | TYR | 326 | 31.994 | 8.694 | 37.781 | 1.00 | 1.11 | 1SG | 2675 |
| ATOM | 2675 | CD2 | TYR | 326 | 31.991 | 6.998 | 36.127 | 1.00 | 1.11 | 1SG | 2676 |
| ATOM | 2676 | CE1 | TYR | 326 | 33.043 | 8.072 | 38.417 | 1.00 | 1.11 | 1SG | 2677 |
| ATOM | 2677 | CE2 | TYR | 326 | 33.040 | 6.371 | 36.758 | 1.00 | 1.11 | 1SG | 2678 |
| ATOM | 2678 | CZ | TYR | 326 | 33.569 | 6.909 | 37.907 | 1.00 | 1.11 | 1SG | 2679 |
| ATOM | 2679 | OH | TYR | 326 | 34.646 | 6.270 | 38.559 | 1.00 | 1.11 | 1SG | 2680 |
| ATOM | 2680 | C | TYR | 326 | 29.057 | 8.802 | 38.033 | 1.00 | 1.11 | 1SG | 2681 |
| ATOM | 2681 | O | TYR | 326 | 29.448 | 9.930 | 38.333 | 1.00 | 1.11 | 1SG | 2682 |
| ATOM | 2682 | N | SER | 327 | 28.612 | 7.910 | 38.935 | 1.00 | 1.25 | 1SG | 2683 |
| ATOM | 2683 | CA | SER | 327 | 28.561 | 8.157 | 40.341 | 1.00 | 1.25 | 1SG | 2684 |
| ATOM | 2684 | CB | SER | 327 | 28.122 | 9.586 | 40.711 | 1.00 | 1.25 | 1SG | 2685 |
| ATOM | 2685 | OG | SER | 327 | 28.093 | 9.746 | 42.122 | 1.00 | 1.25 | 1SG | 2686 |
| ATOM | 2686 | C | SER | 327 | 27.506 | 7.228 | 40.824 | 1.00 | 1.25 | 1SG | 2687 |
| ATOM | 2687 | O | SER | 327 | 26.851 | 6.575 | 40.014 | 1.00 | 1.25 | 1SG | 2688 |
| ATOM | 2688 | N | PRO | 328 | 27.312 | 7.113 | 42.099 | 1.00 | 1.12 | 1SG | 2689 |
| ATOM | 2689 | CA | PRO | 328 | 26.241 | 6.250 | 42.487 | 1.00 | 1.12 | 1SG | 2690 |
| ATOM | 2690 | CD | PRO | 328 | 28.427 | 7.041 | 43.029 | 1.00 | 1.12 | 1SG | 2691 |
| ATOM | 2691 | CB | PRO | 328 | 26.468 | 5.923 | 43.959 | 1.00 | 1.12 | 1SG | 2692 |
| ATOM | 2692 | CG | PRO | 328 | 28.000 | 6.020 | 44.102 | 1.00 | 1.12 | 1SG | 2693 |
| ATOM | 2693 | C | PRO | 328 | 24.962 | 6.943 | 42.177 | 1.00 | 1.12 | 1SG | 2694 |
| ATOM | 2694 | O | PRO | 328 | 24.763 | 8.058 | 42.654 | 1.00 | 1.12 | 1SG | 2695 |
| ATOM | 2695 | N | ASP | 329 | 24.091 | 6.318 | 41.366 | 1.00 | 0.75 | 1SG | 2696 |
| ATOM | 2696 | CA | ASP | 329 | 22.831 | 6.926 | 41.073 | 1.00 | 0.75 | 1SG | 2697 |
| ATOM | 2697 | CB | ASP | 329 | 22.670 | 7.242 | 39.578 | 1.00 | 0.75 | 1SG | 2698 |
| ATOM | 2698 | CG | ASP | 329 | 23.653 | 8.354 | 39.240 | 1.00 | 0.75 | 1SG | 2699 |
| ATOM | 2699 | OD1 | ASP | 329 | 24.098 | 9.058 | 40.185 | 1.00 | 0.75 | 1SG | 2700 |
| ATOM | 2700 | OD2 | ASP | 329 | 23.974 | 8.513 | 38.032 | 1.00 | 0.75 | 1SG | 2701 |
| ATOM | 2701 | C | ASP | 329 | 21.806 | 5.907 | 41.436 | 1.00 | 0.75 | 1SG | 2702 |
| ATOM | 2702 | O | ASP | 329 | 20.618 | 6.074 | 41.163 | 1.00 | 0.75 | 1SG | 2703 |
| ATOM | 2703 | N | GLU | 330 | 22.267 | 4.823 | 42.088 | 1.00 | 0.48 | 1SG | 2704 |
| ATOM | 2704 | CA | GLU | 330 | 21.430 | 3.740 | 42.510 | 1.00 | 0.48 | 1SG | 2705 |
| ATOM | 2705 | CB | GLU | 330 | 22.226 | 2.571 | 43.117 | 1.00 | 0.48 | 1SG | 2706 |
| ATOM | 2706 | CG | GLU | 330 | 22.993 | 1.751 | 42.080 | 1.00 | 0.48 | 1SG | 2707 |
| ATOM | 2707 | CD | GLU | 330 | 21.973 | 0.892 | 41.345 | 1.00 | 0.48 | 1SG | 2708 |
| ATOM | 2708 | OE1 | GLU | 330 | 20.794 | 0.874 | 41.788 | 1.00 | 0.48 | 1SG | 2709 |
| ATOM | 2709 | OE2 | GLU | 330 | 22.357 | 0.240 | 40.337 | 1.00 | 0.48 | 1SG | 2710 |
| ATOM | 2710 | C | GLU | 330 | 20.506 | 4.234 | 43.569 | 1.00 | 0.48 | 1SG | 2711 |
| ATOM | 2711 | O | GLU | 330 | 19.338 | 3.850 | 43.612 | 1.00 | 0.48 | 1SG | 2712 |
| ATOM | 2712 | N | HIS | 331 | 21.009 | 5.123 | 44.444 | 1.00 | 0.45 | 1SG | 2713 |
| ATOM | 2713 | CA | HIS | 331 | 20.216 | 5.588 | 45.540 | 1.00 | 0.45 | 1SG | 2714 |
| ATOM | 2714 | ND1 | HIS | 331 | 22.685 | 7.828 | 45.016 | 1.00 | 0.45 | 1SG | 2715 |
| ATOM | 2715 | CG | HIS | 331 | 21.455 | 7.804 | 45.635 | 1.00 | 0.45 | 1SG | 2716 |
| ATOM | 2716 | CB | HIS | 331 | 20.933 | 6.634 | 46.410 | 1.00 | 0.45 | 1SG | 2717 |
| ATOM | 2717 | NE2 | HIS | 331 | 21.744 | 9.805 | 44.633 | 1.00 | 0.45 | 1SG | 2718 |
| ATOM | 2718 | CD2 | HIS | 331 | 20.893 | 9.019 | 45.390 | 1.00 | 0.45 | 1SG | 2719 |
| ATOM | 2719 | CE1 | HIS | 331 | 22.806 | 9.047 | 44.434 | 1.00 | 0.45 | 1SG | 2720 |
| ATOM | 2720 | C | HIS | 331 | 18.982 | 6.203 | 44.976 | 1.00 | 0.45 | 1SG | 2721 |
| ATOM | 2721 | O | HIS | 331 | 17.895 | 6.021 | 45.522 | 1.00 | 0.45 | 1SG | 2722 |
| ATOM | 2722 | N | LEU | 332 | 19.116 | 6.940 | 43.859 | 1.00 | 0.43 | 1SG | 2723 |
| ATOM | 2723 | CA | LEU | 332 | 17.941 | 7.491 | 43.266 | 1.00 | 0.43 | 1SG | 2724 |
| ATOM | 2724 | CB | LEU | 332 | 18.200 | 8.267 | 41.962 | 1.00 | 0.43 | 1SG | 2725 |
| ATOM | 2725 | CG | LEU | 332 | 19.029 | 9.548 | 42.162 | 1.00 | 0.43 | 1SG | 2726 |
| ATOM | 2726 | CD2 | LEU | 332 | 18.407 | 10.449 | 43.238 | 1.00 | 0.43 | 1SG | 2727 |
| ATOM | 2727 | CD1 | LEU | 332 | 19.254 | 10.279 | 40.828 | 1.00 | 0.43 | 1SG | 2728 |
| ATOM | 2728 | C | LEU | 332 | 17.085 | 6.323 | 42.925 | 1.00 | 0.43 | 1SG | 2729 |
| ATOM | 2729 | O | LEU | 332 | 17.456 | 5.485 | 42.105 | 1.00 | 0.43 | 1SG | 2730 |
| ATOM | 2730 | N | TRP | 333 | 15.904 | 6.241 | 43.567 | 1.00 | 0.40 | 1SG | 2731 |
| ATOM | 2731 | CA | TRP | 333 | 15.044 | 5.120 | 43.351 | 1.00 | 0.40 | 1SG | 2732 |
| ATOM | 2732 | CB | TRP | 333 | 13.702 | 5.219 | 44.098 | 1.00 | 0.40 | 1SG | 2733 |
| ATOM | 2733 | CG | TRP | 333 | 13.830 | 5.236 | 45.605 | 1.00 | 0.40 | 1SG | 2734 |
| ATOM | 2734 | CD2 | TRP | 333 | 13.903 | 4.068 | 46.440 | 1.00 | 0.40 | 1SG | 2735 |
| ATOM | 2735 | CD1 | TRP | 333 | 13.899 | 6.312 | 46.440 | 1.00 | 0.40 | 1SG | 2736 |
| ATOM | 2736 | NE1 | TRP | 333 | 14.010 | 5.890 | 47.743 | 1.00 | 0.40 | 1SG | 2737 |
| ATOM | 2737 | CE2 | TRP | 333 | 14.013 | 4.511 | 47.759 | 1.00 | 0.40 | 1SG | 2738 |
| ATOM | 2738 | CE3 | TRP | 333 | 13.880 | 2.737 | 46.136 | 1.00 | 0.40 | 1SG | 2739 |
| ATOM | 2739 | CZ2 | TRP | 333 | 14.103 | 3.627 | 48.796 | 1.00 | 0.40 | 1SG | 2740 |
| ATOM | 2740 | CZ3 | TRP | 333 | 13.970 | 1.849 | 47.185 | 1.00 | 0.40 | 1SG | 2741 |
| ATOM | 2741 | CH2 | TRP | 333 | 14.079 | 2.285 | 48.490 | 1.00 | 0.40 | 1SG | 2742 |
| ATOM | 2742 | C | TRP | 333 | 14.756 | 5.054 | 41.891 | 1.00 | 0.40 | 1SG | 2743 |
| ATOM | 2743 | O | TRP | 333 | 15.041 | 5.985 | 41.138 | 1.00 | 0.40 | 1SG | 2744 |
| ATOM | 2744 | N | ALA | 334 | 14.174 | 3.927 | 41.450 | 1.00 | 0.47 | 1SG | 2745 |
| ATOM | 2745 | CA | ALA | 334 | 13.889 | 3.771 | 40.058 | 1.00 | 0.47 | 1SG | 2746 |
| ATOM | 2746 | CB | ALA | 334 | 13.133 | 2.470 | 39.740 | 1.00 | 0.47 | 1SG | 2747 |
| ATOM | 2747 | C | ALA | 334 | 12.986 | 4.891 | 39.686 | 1.00 | 0.47 | 1SG | 2748 |
| ATOM | 2748 | O | ALA | 334 | 13.160 | 5.532 | 38.652 | 1.00 | 0.47 | 1SG | 2749 |
| ATOM | 2749 | N | THR | 335 | 11.997 | 5.163 | 40.550 | 1.00 | 0.67 | 1SG | 2750 |
| ATOM | 2750 | CA | THR | 335 | 11.086 | 6.223 | 40.261 | 1.00 | 0.67 | 1SG | 2751 |
| ATOM | 2751 | CB | THR | 335 | 9.670 | 5.902 | 40.682 | 1.00 | 0.67 | 1SG | 2752 |
| ATOM | 2752 | OG1 | THR | 335 | 8.805 | 6.982 | 40.365 | 1.00 | 0.67 | 1SG | 2753 |
| ATOM | 2753 | CG2 | THR | 335 | 9.619 | 5.575 | 42.186 | 1.00 | 0.67 | 1SG | 2754 |
| ATOM | 2754 | C | THR | 335 | 11.556 | 7.444 | 40.978 | 1.00 | 0.67 | 1SG | 2755 |
| ATOM | 2755 | O | THR | 335 | 11.534 | 7.526 | 42.206 | 1.00 | 0.67 | 1SG | 2756 |
| ATOM | 2756 | N | LEU | 336 | 12.047 | 8.429 | 40.209 | 1.00 | 0.80 | 1SG | 2757 |
| ATOM | 2757 | CA | LEU | 336 | 12.446 | 9.652 | 40.829 | 1.00 | 0.80 | 1SG | 2758 |
| ATOM | 2758 | CB | LEU | 336 | 13.930 | 9.987 | 40.601 | 1.00 | 0.80 | 1SG | 2759 |
| ATOM | 2759 | CG | LEU | 336 | 14.397 | 11.297 | 41.256 | 1.00 | 0.80 | 1SG | 2760 |
| ATOM | 2760 | CD2 | LEU | 336 | 15.824 | 11.663 | 40.813 | 1.00 | 0.80 | 1SG | 2761 |
| ATOM | 2761 | CD1 | LEU | 336 | 14.254 | 11.226 | 42.786 | 1.00 | 0.80 | 1SG | 2762 |
| ATOM | 2762 | C | LEU | 336 | 11.628 | 10.715 | 40.175 | 1.00 | 0.80 | 1SG | 2763 |
| ATOM | 2763 | O | LEU | 336 | 12.065 | 11.344 | 39.214 | 1.00 | 0.80 | 1SG | 2764 |
| ATOM | 2764 | N | GLN | 337 | 10.405 | 10.944 | 40.676 | 1.00 | 0.75 | 1SG | 2765 |
| ATOM | 2765 | CA | GLN | 337 | 9.597 | 11.958 | 40.083 | 1.00 | 0.75 | 1SG | 2766 |
| ATOM | 2766 | CB | GLN | 337 | 8.154 | 12.034 | 40.610 | 1.00 | 0.75 | 1SG | 2767 |
| ATOM | 2767 | CG | GLN | 337 | 7.411 | 13.329 | 40.272 | 1.00 | 0.75 | 1SG | 2768 |
| ATOM | 2768 | CD | GLN | 337 | 7.659 | 14.319 | 41.413 | 1.00 | 0.75 | 1SG | 2769 |
| ATOM | 2769 | OE1 | GLN | 337 | 8.414 | 14.024 | 42.338 | 1.00 | 0.75 | 1SG | 2770 |
| ATOM | 2770 | NE2 | GLN | 337 | 7.009 | 15.511 | 41.357 | 1.00 | 0.75 | 1SG | 2771 |
| ATOM | 2771 | C | GLN | 337 | 10.200 | 13.274 | 40.365 | 1.00 | 0.75 | 1SG | 2772 |
| ATOM | 2772 | O | GLN | 337 | 10.118 | 14.189 | 39.548 | 1.00 | 0.75 | 1SG | 2773 |
| ATOM | 2773 | N | ARG | 338 | 10.836 | 13.405 | 41.537 | 1.00 | 0.55 | 1SG | 2774 |
| ATOM | 2774 | CA | ARG | 338 | 11.233 | 14.719 | 41.909 | 1.00 | 0.55 | 1SG | 2775 |
| ATOM | 2775 | CB | ARG | 338 | 11.475 | 14.881 | 43.418 | 1.00 | 0.55 | 1SG | 2776 |
| ATOM | 2776 | CG | ARG | 338 | 11.321 | 16.333 | 43.879 | 1.00 | 0.55 | 1SG | 2777 |
| ATOM | 2777 | CD | ARG | 338 | 9.871 | 16.815 | 43.808 | 1.00 | 0.55 | 1SG | 2778 |
| ATOM | 2778 | NE | ARG | 338 | 9.827 | 18.226 | 44.283 | 1.00 | 0.55 | 1SG | 2779 |
| ATOM | 2779 | CZ | ARG | 338 | 8.719 | 18.683 | 44.938 | 1.00 | 0.55 | 1SG | 2780 |
| ATOM | 2780 | NH1 | ARG | 338 | 7.677 | 17.636 | 45.187 | 1.00 | 0.55 | 1SG | 2781 |
| ATOM | 2781 | NH2 | ARG | 338 | 8.650 | 19.984 | 45.344 | 1.00 | 0.55 | 1SG | 2782 |
| ATOM | 2782 | C | ARG | 338 | 12.472 | 15.111 | 41.185 | 1.00 | 0.55 | 1SG | 2783 |
| ATOM | 2783 | O | ARG | 338 | 13.577 | 14.995 | 41.709 | 1.00 | 0.55 | 1SG | 2784 |
| ATOM | 2784 | N | ALA | 339 | 12.296 | 15.571 | 39.935 | 1.00 | 0.40 | 1SG | 2785 |
| ATOM | 2785 | CA | ALA | 339 | 13.359 | 16.168 | 39.200 | 1.00 | 0.40 | 1SG | 2786 |
| ATOM | 2786 | CB | ALA | 339 | 13.392 | 15.764 | 37.718 | 1.00 | 0.40 | 1SG | 2787 |
| ATOM | 2787 | C | ALA | 339 | 12.965 | 17.597 | 39.265 | 1.00 | 0.40 | 1SG | 2788 |
| ATOM | 2788 | O | ALA | 339 | 12.271 | 18.106 | 38.386 | 1.00 | 0.40 | 1SG | 2789 |
| ATOM | 2789 | N | ARG | 340 | 13.387 | 18.273 | 40.345 | 1.00 | 0.63 | 1SG | 2790 |
| ATOM | 2790 | CA | ARG | 340 | 12.941 | 19.609 | 40.555 | 1.00 | 0.63 | 1SG | 2791 |
| ATOM | 2791 | CB | ARG | 340 | 12.053 | 19.738 | 41.810 | 1.00 | 0.63 | 1SG | 2792 |
| ATOM | 2792 | CG | ARG | 340 | 11.414 | 21.114 | 42.012 | 1.00 | 0.63 | 1SG | 2793 |
| ATOM | 2793 | CD | ARG | 340 | 10.679 | 21.243 | 43.346 | 1.00 | 0.63 | 1SG | 2794 |
| ATOM | 2794 | NE | ARG | 340 | 10.109 | 22.617 | 43.414 | 1.00 | 0.63 | 1SG | 2795 |
| ATOM | 2795 | CZ | ARG | 340 | 9.742 | 23.145 | 44.619 | 1.00 | 0.63 | 1SG | 2796 |
| ATOM | 2796 | NH1 | ARG | 340 | 9.923 | 22.426 | 45.765 | 1.00 | 0.63 | 1SG | 2797 |
| ATOM | 2797 | NH2 | ARG | 340 | 9.205 | 24.398 | 44.680 | 1.00 | 0.63 | 1SG | 2798 |
| ATOM | 2798 | C | ARG | 340 | 14.146 | 20.457 | 40.760 | 1.00 | 0.63 | 1SG | 2799 |
| ATOM | 2799 | O | ARG | 340 | 15.277 | 19.977 | 40.788 | 1.00 | 0.63 | 1SG | 2800 |
| ATOM | 2800 | N | TRP | 341 | 13.907 | 21.769 | 40.867 | 1.00 | 1.01 | 1SG | 2801 |
| ATOM | 2801 | CA | TRP | 341 | 14.916 | 22.749 | 41.102 | 1.00 | 1.01 | 1SG | 2802 |
| ATOM | 2802 | CB | TRP | 341 | 15.754 | 23.074 | 39.850 | 1.00 | 1.01 | 1SG | 2803 |
| ATOM | 2803 | CG | TRP | 341 | 16.897 | 24.035 | 40.088 | 1.00 | 1.01 | 1SG | 2804 |
| ATOM | 2804 | CD2 | TRP | 341 | 18.189 | 23.619 | 40.554 | 1.00 | 1.01 | 1SG | 2805 |
| ATOM | 2805 | CD1 | TRP | 341 | 16.962 | 25.390 | 39.931 | 1.00 | 1.01 | 1SG | 2806 |
| ATOM | 2806 | NE1 | TRP | 341 | 18.214 | 25.843 | 40.272 | 1.00 | 1.01 | 1SG | 2807 |
| ATOM | 2807 | CE2 | TRP | 341 | 18.980 | 24.763 | 40.656 | 1.00 | 1.01 | 1SG | 2808 |
| ATOM | 2808 | CE3 | TRP | 341 | 18.676 | 22.383 | 40.869 | 1.00 | 1.01 | 1SG | 2809 |
| ATOM | 2809 | CZ2 | TRP | 341 | 20.217 | 24.686 | 41.077 | 1.00 | 1.01 | 1SG | 2810 |
| ATOM | 2810 | CZ3 | TRP | 341 | 19.983 | 22.310 | 41.294 | 1.00 | 1.01 | 1SG | 2811 |
| ATOM | 2811 | CH2 | TRP | 341 | 20.769 | 23.439 | 41.395 | 1.00 | 1.01 | 1SG | 2812 |
| ATOM | 2812 | C | TRP | 341 | 14.095 | 23.941 | 41.425 | 1.00 | 1.01 | 1SG | 2813 |
| ATOM | 2813 | O | TRP | 341 | 13.328 | 23.932 | 42.387 | 1.00 | 1.01 | 1SG | 2814 |
| ATOM | 2814 | N | MET | 342 | 14.259 | 25.014 | 40.641 | 1.00 | 1.01 | 1SG | 2815 |
| ATOM | 2815 | CA | MET | 342 | 13.373 | 26.120 | 40.788 | 1.00 | 1.01 | 1SG | 2816 |
| ATOM | 2816 | CB | MET | 342 | 14.084 | 27.482 | 40.715 | 1.00 | 1.01 | 1SG | 2817 |
| ATOM | 2817 | CG | MET | 342 | 13.145 | 28.680 | 40.871 | 1.00 | 1.01 | 1SG | 2818 |
| ATOM | 2818 | SD | MET | 342 | 13.971 | 30.298 | 40.786 | 1.00 | 1.01 | 1SG | 2819 |
| ATOM | 2819 | CE | MET | 342 | 12.459 | 31.264 | 41.063 | 1.00 | 1.01 | 1SG | 2820 |
| ATOM | 2820 | C | MET | 342 | 12.493 | 26.007 | 39.590 | 1.00 | 1.01 | 1SG | 2821 |
| ATOM | 2821 | O | MET | 342 | 12.579 | 26.824 | 38.675 | 1.00 | 1.01 | 1SG | 2822 |
| ATOM | 2822 | N | PRO | 343 | 11.650 | 24.996 | 39.611 | 1.00 | 0.65 | 1SG | 2823 |
| ATOM | 2823 | CA | PRO | 343 | 10.804 | 24.676 | 38.489 | 1.00 | 0.65 | 1SG | 2824 |
| ATOM | 2824 | CD | PRO | 343 | 11.042 | 24.580 | 40.865 | 1.00 | 0.65 | 1SG | 2825 |
| ATOM | 2825 | CB | PRO | 343 | 9.810 | 23.640 | 39.004 | 1.00 | 0.65 | 1SG | 2826 |
| ATOM | 2826 | CG | PRO | 343 | 9.659 | 24.023 | 40.486 | 1.00 | 0.65 | 1SG | 2827 |
| ATOM | 2827 | C | PRO | 343 | 10.098 | 25.907 | 38.033 | 1.00 | 0.65 | 1SG | 2828 |
| ATOM | 2828 | O | PRO | 343 | 9.506 | 26.596 | 38.860 | 1.00 | 0.65 | 1SG | 2829 |
| ATOM | 2829 | N | GLY | 344 | 10.147 | 26.187 | 36.719 | 1.00 | 0.31 | 1SG | 2830 |
| ATOM | 2830 | CA | GLY | 344 | 9.550 | 27.371 | 36.182 | 1.00 | 0.31 | 1SG | 2831 |
| ATOM | 2831 | C | GLY | 344 | 10.077 | 27.489 | 34.791 | 1.00 | 0.31 | 1SG | 2832 |
| ATOM | 2832 | O | GLY | 344 | 9.818 | 26.645 | 33.938 | 1.00 | 0.31 | 1SG | 2833 |
| ATOM | 2833 | N | SER | 345 | 10.802 | 28.581 | 34.510 | 1.00 | 0.29 | 1SG | 2834 |
| ATOM | 2834 | CA | SER | 345 | 11.425 | 28.729 | 33.231 | 1.00 | 0.29 | 1SG | 2835 |
| ATOM | 2835 | CB | SER | 345 | 12.014 | 30.134 | 33.019 | 1.00 | 0.29 | 1SG | 2836 |
| ATOM | 2836 | OG | SER | 345 | 10.978 | 31.105 | 33.039 | 1.00 | 0.29 | 1SG | 2837 |
| ATOM | 2837 | C | SER | 345 | 12.559 | 27.751 | 33.194 | 1.00 | 0.29 | 1SG | 2838 |
| ATOM | 2838 | O | SER | 345 | 12.919 | 27.230 | 32.139 | 1.00 | 0.29 | 1SG | 2839 |
| ATOM | 2839 | N | VAL | 346 | 13.126 | 27.466 | 34.383 | 1.00 | 0.52 | 1SG | 2840 |
| ATOM | 2840 | CA | VAL | 346 | 14.309 | 26.667 | 34.550 | 1.00 | 0.52 | 1SG | 2841 |
| ATOM | 2841 | CB | VAL | 346 | 14.792 | 26.583 | 35.978 | 1.00 | 0.52 | 1SG | 2842 |
| ATOM | 2842 | CG1 | VAL | 346 | 14.018 | 25.507 | 36.752 | 1.00 | 0.52 | 1SG | 2843 |
| ATOM | 2843 | CG2 | VAL | 346 | 16.313 | 26.378 | 35.963 | 1.00 | 0.52 | 1SG | 2844 |
| ATOM | 2844 | C | VAL | 346 | 14.146 | 25.278 | 33.989 | 1.00 | 0.52 | 1SG | 2845 |
| ATOM | 2845 | O | VAL | 346 | 15.168 | 24.679 | 33.660 | 1.00 | 0.52 | 1SG | 2846 |
| ATOM | 2846 | N | PRO | 347 | 12.981 | 24.692 | 33.830 | 1.00 | 0.58 | 1SG | 2847 |
| ATOM | 2847 | CA | PRO | 347 | 12.951 | 23.369 | 33.269 | 1.00 | 0.58 | 1SG | 2848 |
| ATOM | 2848 | CD | PRO | 347 | 11.862 | 24.889 | 34.738 | 1.00 | 0.58 | 1SG | 2849 |
| ATOM | 2849 | CB | PRO | 347 | 11.505 | 22.899 | 33.384 | 1.00 | 0.58 | 1SG | 2850 |
| ATOM | 2850 | CG | PRO | 347 | 11.018 | 23.605 | 34.661 | 1.00 | 0.58 | 1SG | 2851 |
| ATOM | 2851 | C | PRO | 347 | 13.518 | 23.360 | 31.893 | 1.00 | 0.58 | 1SG | 2852 |
| ATOM | 2852 | O | PRO | 347 | 13.800 | 22.282 | 31.371 | 1.00 | 0.58 | 1SG | 2853 |
| ATOM | 2853 | N | ASN | 348 | 13.676 | 24.541 | 31.278 | 1.00 | 0. 42 | 1SG | 2854 |
| ATOM | 2854 | CA | ASN | 348 | 14.273 | 24.586 | 29.984 | 1.00 | 0.42 | 1SG | 2855 |
| ATOM | 2855 | CB | ASN | 348 | 14.364 | 26.014 | 29.417 | 1.00 | 0.42 | 1SG | 2856 |
| ATOM | 2856 | CG | ASN | 348 | 12.953 | 26.516 | 29.150 | 1.00 | 0.42 | 1SG | 2857 |
| ATOM | 2857 | OD1 | ASN | 348 | 12.013 | 25.733 | 29.027 | 1.00 | 0.42 | 1SG | 2858 |
| ATOM | 2858 | ND2 | ASN | 348 | 12.797 | 27.863 | 29.048 | 1.00 | 0.42 | 1SG | 2859 |
| ATOM | 2859 | C | ASN | 348 | 15.674 | 24.080 | 30.136 | 1.00 | 0.42 | 1SG | 8260 |
| ATOM | 2860 | O | ASN | 348 | 16.170 | 23.352 | 29.281 | 1.00 | 0.42 | 1SG | 2861 |
| ATOM | 2861 | N | HIS | 349 | 16.339 | 24.421 | 31.257 | 1.00 | 0.48 | 1SG | 2862 |
| ATOM | 2862 | CA | HIS | 349 | 17.725 | 24.080 | 31.430 | 1.00 | 0.48 | 1SG | 2863 |
| ATOM | 2863 | ND1 | HIS | 349 | 19.358 | 22.875 | 34.147 | 1.00 | 0.48 | 1SG | 8264 |
| ATOM | 2864 | CG | HIS | 349 | 18.423 | 23.865 | 33.914 | 1.00 | 0.48 | 1SG | 2865 |
| ATOM | 2865 | CB | HIS | 349 | 18.403 | 24.710 | 32.674 | 1.00 | 0.48 | 1SG | 2866 |
| ATOM | 2866 | NE2 | HIS | 349 | 18.009 | 22.904 | 35.914 | 1.00 | 0.48 | 1SG | 2867 |
| ATOM | 2867 | CD2 | HIS | 349 | 17.613 | 23.868 | 35.004 | 1.00 | 0.48 | 1SG | 2868 |
| ATOM | 2868 | CE1 | HIS | 349 | 19.060 | 22.334 | 35.355 | 1.00 | 0.48 | 1SG | 2869 |
| ATOM | 2869 | C | HIS | 349 | 17.931 | 22.592 | 31.447 | 1.00 | 0.48 | 1SG | 2870 |
| ATOM | 2870 | O | HIS | 349 | 18.985 | 22.149 | 30.994 | 1.00 | 0.48 | 1SG | 2871 |
| ATOM | 2871 | N | PRO | 350 | 17.032 | 21.764 | 31.918 | 1.00 | 0.62 | 1SG | 2872 |
| ATOM | 2872 | CA | PRO | 350 | 17.331 | 20.359 | 31.911 | 1.00 | 0.62 | 1SG | 2873 |
| ATOM | 2873 | CD | PRO | 350 | 16.109 | 22.106 | 32.989 | 1.00 | 0.62 | 1SG | 2874 |
| ATOM | 2874 | CB | PRO | 350 | 16.303 | 19.700 | 32.829 | 1.00 | 0.62 | 1SG | 2875 |
| ATOM | 2875 | CG | PRO | 350 | 15.913 | 20.821 | 33.804 | 1.00 | 0.62 | 1SG | 2876 |
| ATOM | 2876 | C | PRO | 350 | 17.295 | 19.838 | 30.514 | 1.00 | 0.62 | 1SG | 2877 |
| ATOM | 2877 | O | PRO | 350 | 17.575 | 18.657 | 30.318 | 1.00 | 0.62 | 1SG | 2878 |
| ATOM | 2878 | N | LYS | 351 | 16.964 | 20.700 | 29.534 | 1.00 | 0.60 | 1SG | 2879 |
| ATOM | 2879 | CA | LYS | 351 | 16.826 | 20.275 | 28.173 | 1.00 | 0.60 | 1SG | 2880 |
| ATOM | 2880 | CB | LYS | 351 | 16.524 | 21.451 | 27.219 | 1.00 | 0.60 | 1SG | 2881 |
| ATOM | 2881 | CG | LYS | 351 | 17.636 | 22.506 | 27.138 | 1.00 | 0.60 | 1SG | 2882 |
| ATOM | 2882 | CD | LYS | 351 | 17.464 | 23.546 | 26.030 | 1.00 | 0.60 | 1SG | 2883 |
| ATOM | 2833 | CE | LYS | 351 | 18.587 | 24.586 | 26.018 | 1.00 | 0.60 | 1SG | 2884 |
| ATOM | 2884 | NZ | LYS | 351 | 18.365 | 25.567 | 24.933 | 1.00 | 0.60 | 1SG | 2885 |
| ATOM | 2885 | C | LYS | 351 | 18.092 | 19.630 | 27.712 | 1.00 | 0.60 | 1SG | 2886 |
| ATOM | 2886 | O | LYS | 351 | 18.081 | 18.501 | 27.225 | 1.00 | 0.60 | 1SG | 2887 |
| ATOM | 2887 | N | TYR | 352 | 19.233 | 20.314 | 27.884 | 1.00 | 0.49 | 1SG | 2888 |
| ATOM | 2888 | CA | TYR | 352 | 20.452 | 19.759 | 27.391 | 1.00 | 0.49 | 1SG | 2889 |
| ATOM | 2889 | CB | TYR | 352 | 21.653 | 20.710 | 27.530 | 1.00 | 0.49 | 1SG | 2890 |
| ATOM | 2890 | CG | TYR | 352 | 22.832 | 19.985 | 26.980 | 1.00 | 0.49 | 1SG | 2891 |
| ATOM | 2891 | CD1 | TYR | 352 | 23.063 | 19.952 | 25.621 | 1.00 | 0.49 | 1SG | 2892 |
| ATOM | 2892 | CD2 | TYR | 352 | 23.703 | 19.333 | 27.819 | 1.00 | 0.49 | 1SG | 2893 |
| ATOM | 2893 | CE1 | TYR | 352 | 24.150 | 19.280 | 25.113 | 1.00 | 0.49 | 1SG | 2894 |
| ATOM | 2894 | CE2 | TYR | 352 | 24.791 | 18.659 | 27.317 | 1.00 | 0.49 | 1SG | 2895 |
| ATOM | 2895 | CZ | TYR | 352 | 25.015 | 18.632 | 25.962 | 1.00 | 0.49 | 1SG | 2896 |
| ATOM | 2896 | OH | TYR | 352 | 26.131 | 17.942 | 25.443 | 1.00 | 0.49 | 1SG | 2897 |
| ATOM | 2897 | C | TYR | 352 | 20.758 | 18.541 | 28.189 | 1.00 | 0.49 | 1SG | 2898 |
| ATOM | 2898 | O | TYR | 352 | 21.189 | 17.522 | 27.651 | 1.00 | 0.49 | 1SG | 2899 |
| ATOM | 2899 | N | ASP | 353 | 20.517 | 18.615 | 29.507 | 1.00 | 0.49 | 1SG | 2900 |
| ATOM | 2900 | CA | ASP | 353 | 20.916 | 17.529 | 30.342 | 1.00 | 0.49 | 1SG | 2901 |
| ATOM | 2901 | CB | ASP | 353 | 20.569 | 17.779 | 31.818 | 1.00 | 0.49 | 1SG | 2902 |
| ATOM | 2902 | CG | ASP | 353 | 21.401 | 18.963 | 32.292 | 1.00 | 0.49 | 1SG | 2903 |
| ATOM | 2903 | OD1 | ASP | 353 | 22.293 | 19.405 | 31.519 | 1.00 | 0.49 | 1SG | 2904 |
| ATOM | 2904 | OD2 | ASP | 353 | 21.152 | 19.447 | 33.428 | 1.00 | 0.49 | 1SG | 2905 |
| ATOM | 2905 | C | ASP | 353 | 20.216 | 16.273 | 29.928 | 1.00 | 0.49 | 1SG | 2906 |
| ATOM | 2906 | O | ASP | 353 | 20.860 | 15.292 | 29.560 | 1.00 | 0.49 | 1SG | 2907 |
| ATOM | 2907 | N | ILE | 354 | 18.871 | 16.273 | 29.962 | 1.00 | 0.71 | 1SG | 2908 |
| ATOM | 2908 | CA | ILE | 354 | 18.151 | 15.069 | 29.657 | 1.00 | 0.71 | 1SG | 2909 |
| ATOM | 2909 | CB | ILE | 354 | 16.739 | 15.067 | 30.144 | 1.00 | 0.71 | 1SG | 2910 |
| ATOM | 2910 | CG2 | ILE | 354 | 16.773 | 15.237 | 31.672 | 1.00 | 0.71 | 1SG | 2911 |
| ATOM | 2911 | CG1 | ILE | 354 | 15.912 | 16.134 | 29.431 | 1.00 | 0.71 | 1SG | 2912 |
| ATOM | 2912 | CD1 | ILE | 354 | 14.426 | 15.926 | 29.674 | 1.00 | 0.71 | 1SG | 2913 |
| ATOM | 2913 | C | ILE | 354 | 18.138 | 14.799 | 28.183 | 1.00 | 0.71 | 1SG | 2914 |
| ATOM | 2914 | O | ILE | 354 | 18.213 | 13.643 | 27.770 | 1.00 | 0.71 | 1SG | 2915 |
| ATOM | 2915 | N | SER | 355 | 18.062 | 15.869 | 27.362 | 1.00 | 0.84 | 1SG | 2916 |
| ATOM | 2916 | CA | SER | 355 | 17.985 | 15.776 | 25.926 | 1.00 | 0.84 | 1SG | 2917 |
| ATOM | 2917 | CB | SER | 355 | 18.845 | 14.655 | 25.312 | 1.00 | 0.84 | 1SG | 2918 |
| ATOM | 2918 | OG | SER | 355 | 20.220 | 14.902 | 25.568 | 1.00 | 0.84 | 1SG | 2919 |
| ATOM | 2919 | C | SER | 355 | 16.563 | 15.569 | 25.494 | 1.00 | 0.84 | 1SG | 2920 |
| ATOM | 2920 | O | SER | 355 | 16.281 | 14.818 | 24.561 | 1.00 | 0.84 | 1SG | 2921 |
| ATOM | 2921 | N | ASP | 356 | 15.624 | 16.249 | 26.179 | 1.00 | 0.69 | 1SG | 2922 |
| ATOM | 2922 | CA | ASP | 356 | 14.225 | 16.219 | 25.847 | 1.00 | 0.69 | 1SG | 2923 |
| ATOM | 2923 | CB | ASP | 356 | 13.282 | 16.459 | 27.034 | 1.00 | 0.69 | 1SG | 2924 |
| ATOM | 2924 | CG | ASP | 356 | 13.592 | 17.852 | 27.597 | 1.00 | 0.69 | 1SG | 2925 |
| ATOM | 2925 | OD1 | ASP | 356 | 14.797 | 18.206 | 27.690 | 1.00 | 0.69 | 1SG | 2926 |
| ATOM | 2926 | OD2 | ASP | 356 | 12.631 | 18.591 | 27.936 | 1.00 | 0.69 | 1SG | 2927 |
| ATOM | 2927 | C | ASP | 356 | 13.934 | 17.359 | 24.926 | 1.00 | 0.69 | 1SG | 2928 |
| ATOM | 2928 | O | ASP | 356 | 14.752 | 18.262 | 24.758 | 1.00 | 0.69 | 1SG | 2929 |
| ATOM | 2929 | N | MET | 357 | 12.732 | 17.337 | 24.311 | 1.00 | 0.38 | 1SG | 2930 |
| ATOM | 2930 | CA | MET | 357 | 12.330 | 18.412 | 23.453 | 1.00 | 0.38 | 1SG | 2931 |
| ATOM | 2931 | CB | MET | 357 | 11.057 | 18.076 | 22.648 | 1.00 | 0.38 | 1SG | 2932 |
| ATOM | 2932 | CG | MET | 357 | 10.743 | 19.021 | 21.487 | 1.00 | 0.38 | 1SG | 2933 |
| ATOM | 2933 | SD | MET | 357 | 9.982 | 20.595 | 21.967 | 1.00 | 0.38 | 1SG | 2934 |
| ATOM | 2934 | CE | MET | 357 | 8.406 | 19.833 | 22.446 | 1.00 | 0.38 | 1SG | 2935 |
| ATOM | 2935 | C | MET | 357 | 12.081 | 19.583 | 24.361 | 1.00 | 0.38 | 1SG | 2936 |
| ATOM | 2936 | O | MET | 357 | 11.287 | 19.507 | 25.294 | 1.00 | 0.38 | 1SG | 2937 |
| ATOM | 2937 | N | THR | 358 | 12.811 | 20.688 | 24.119 | 1.00 | 0.27 | 1SG | 2938 |
| ATOM | 2938 | CA | THR | 358 | 12.835 | 21.880 | 24.926 | 1.00 | 0.27 | 1SG | 2939 |
| ATOM | 2939 | CB | THR | 358 | 13.983 | 22.767 | 24.543 | 1.00 | 0.27 | 1SG | 2940 |
| ATOM | 2940 | OG1 | THR | 358 | 13.795 | 23.257 | 23.224 | 1.00 | 0.27 | 1SG | 2941 |
| ATOM | 2941 | CG2 | THR | 358 | 15.279 | 21.941 | 24.595 | 1.00 | 0.27 | 1SG | 2942 |
| ATOM | 2942 | C | THR | 358 | 11.603 | 22.732 | 24.816 | 1.00 | 0.27 | 1SG | 2943 |
| ATOM | 2943 | O | THR | 358 | 11.173 | 23.325 | 25.805 | 1.00 | 0.27 | 1SG | 2944 |
| ATOM | 2944 | N | SER | 359 | 11.016 | 22.830 | 23.610 | 1.00 | 0.41 | 1SG | 2945 |
| ATOM | 2945 | CA | SER | 359 | 9.960 | 23.770 | 23.338 | 1.00 | 0.41 | 1SG | 2946 |
| ATOM | 2946 | CB | SER | 359 | 9.481 | 23.744 | 21.877 | 1.00 | 0.41 | 1SG | 2947 |
| ATOM | 2947 | OG | SER | 359 | 8.447 | 24.699 | 21.685 | 1.00 | 0.41 | 1SG | 2948 |
| ATOM | 2948 | C | SER | 359 | 8.763 | 23.550 | 24.204 | 1.00 | 0.41 | 1SG | 2949 |
| ATOM | 2949 | O | SER | 359 | 8.568 | 22.484 | 24.786 | 1.00 | 0.41 | 1SG | 2950 |
| ATOM | 2950 | N | ILE | 360 | 7.937 | 24.614 | 24.322 | 1.00 | 0.40 | 1SG | 2951 |
| ATOM | 2951 | CA | ILE | 360 | 6.719 | 24.561 | 25.075 | 1.00 | 0.40 | 1SG | 2952 |
| ATOM | 2952 | CB | ILE | 360 | 6.769 | 25.389 | 26.326 | 1.00 | 0.40 | 1SG | 2953 |
| ATOM | 2953 | CG2 | ILE | 360 | 5.373 | 25.376 | 26.967 | 1.00 | 0.40 | 1SG | 2954 |
| ATOM | 2954 | CG1 | ILE | 360 | 7.878 | 24.872 | 27.257 | 1.00 | 0.40 | 1SG | 2955 |
| ATOM | 2955 | CD1 | ILE | 360 | 7.687 | 23.420 | 27.695 | 1.00 | 0.40 | 1SG | 2956 |
| ATOM | 2956 | C | ILE | 360 | 5.649 | 25.130 | 24.204 | 1.00 | 0.40 | 1SG | 2957 |
| ATOM | 2957 | O | ILE | 360 | 5.814 | 26.196 | 23.614 | 1.00 | 0.40 | 1SG | 2958 |
| ATOM | 2958 | N | ALA | 361 | 4.511 | 24.421 | 24.092 | 1.00 | 0.47 | 1SG | 2959 |
| ATOM | 2959 | CA | ALA | 361 | 3.458 | 24.932 | 23.270 | 1.00 | 0.47 | 1SG | 2960 |
| ATOM | 2960 | CB | ALA | 361 | 2.968 | 23.931 | 22.213 | 1.00 | 0.47 | 1SG | 2961 |
| ATOM | 2961 | C | ALA | 361 | 2.305 | 25.222 | 24.161 | 1.00 | 0.47 | 1SG | 2962 |
| ATOM | 2962 | O | ALA | 361 | 1.822 | 24.333 | 24.859 | 1.00 | 0.47 | 1SG | 2963 |
| ATOM | 2963 | N | ARG | 362 | 1.847 | 26.489 | 24.147 | 1.00 | 0.75 | 1SG | 2964 |
| ATOM | 2964 | CA | ARG | 362 | 0.733 | 26.935 | 24.929 | 1.00 | 0.75 | 1SG | 2965 |
| ATOM | 2965 | CB | ARG | 362 | −0.638 | 26.473 | 24.407 | 1.00 | 0.75 | 1SG | 2966 |
| ATOM | 2966 | CG | ARG | 362 | −0.803 | 24.953 | 24.387 | 1.00 | 0.75 | 1SG | 2967 |
| ATOM | 2967 | CD | ARG | 362 | −2.256 | 24.494 | 24.301 | 1.00 | 0.75 | 1SG | 2968 |
| ATOM | 2968 | NE | ARG | 362 | −2.909 | 24.863 | 25.589 | 1.00 | 0.75 | 1SG | 2969 |
| ATOM | 2969 | CZ | ARG | 362 | −2.862 | 24.004 | 26.648 | 1.00 | 0.75 | 1SG | 2970 |
| ATOM | 2970 | NH1 | ARG | 362 | −2.216 | 22.807 | 26.527 | 1.00 | 0.75 | 1SG | 2971 |
| ATOM | 2971 | NH2 | ARG | 362 | −3.462 | 24.339 | 27.828 | 1.00 | 0.75 | 1SG | 2972 |
| ATOM | 2972 | C | ARG | 362 | 0.867 | 26.429 | 26.321 | 1.00 | 0.75 | 1SG | 2973 |
| ATOM | 2973 | O | ARG | 362 | −0.078 | 25.873 | 26.870 | 1.00 | 0.75 | 1SG | 2974 |
| ATOM | 2974 | N | LEU | 363 | 2.056 | 26.609 | 26.915 | 1.00 | 0.73 | 1SG | 2975 |
| ATOM | 2975 | CA | LEU | 363 | 2.353 | 26.311 | 28.288 | 1.00 | 0.73 | 1SG | 2976 |
| ATOM | 2976 | CB | LEU | 363 | 1.198 | 26.198 | 29.343 | 1.00 | 0.73 | 1SG | 2977 |
| ATOM | 2977 | CG | LEU | 363 | −0.162 | 26.964 | 29.342 | 1.00 | 0.73 | 1SG | 2978 |
| ATOM | 2978 | CD2 | LEU | 363 | −0.287 | 28.149 | 28.390 | 1.00 | 0.73 | 1SG | 2979 |
| ATOM | 2979 | CD1 | LEU | 363 | −0.676 | 27.176 | 30.775 | 1.00 | 0.73 | 1SG | 2980 |
| ATOM | 2980 | C | LEU | 363 | 2.850 | 24.901 | 28.413 | 1.00 | 0.73 | 1SG | 2981 |
| ATOM | 2981 | O | LEU | 363 | 3.596 | 24.607 | 29.345 | 1.00 | 0.73 | 1SG | 2982 |
| ATOM | 2982 | N | VAL | 364 | 2.476 | 23.986 | 27.493 | 1.00 | 0.67 | 1SG | 2983 |
| ATOM | 2983 | CA | VAL | 364 | 2.726 | 22.591 | 27.763 | 1.00 | 0.67 | 1SG | 2984 |
| ATOM | 2984 | CB | VAL | 364 | 1.586 | 21.713 | 27.323 | 1.00 | 0.67 | 1SG | 2985 |
| ATOM | 2985 | CG1 | VAL | 364 | 1.930 | 20.247 | 27.639 | 1.00 | 0.67 | 1SG | 2986 |
| ATOM | 2986 | CG2 | VAL | 364 | 0.292 | 22.213 | 27.999 | 1.00 | 0.67 | 1SG | 2987 |
| ATOM | 2987 | C | VAL | 364 | 3.966 | 22.063 | 27.121 | 1.00 | 0.67 | 1SG | 2988 |
| ATOM | 2988 | O | VAL | 364 | 4.235 | 22.304 | 25.948 | 1.00 | 0.67 | 1SG | 2989 |
| ATOM | 2989 | N | LYS | 365 | 4.747 | 21.285 | 27.902 | 1.00 | 0.90 | 1SG | 2990 |
| ATOM | 2990 | CA | LYS | 365 | 5.961 | 20.700 | 27.414 | 1.00 | 0.90 | 1SG | 2991 |
| ATOM | 2991 | CB | LYS | 365 | 7.080 | 20.548 | 28.466 | 1.00 | 0.90 | 1SG | 2992 |
| ATOM | 2992 | CG | LYS | 365 | 6.586 | 20.301 | 29.889 | 1.00 | 0.90 | 1SG | 2993 |
| ATOM | 2993 | CD | LYS | 365 | 5.893 | 21.533 | 30.469 | 1.00 | 0.90 | 1SG | 2994 |
| ATOM | 2994 | CE | LYS | 365 | 6.787 | 22.775 | 30.434 | 1.00 | 0.90 | 1SG | 2995 |
| ATOM | 2995 | NZ | LYS | 365 | 5.965 | 24.002 | 30.505 | 1.00 | 0.90 | 1SG | 2996 |
| ATOM | 2996 | C | LYS | 365 | 5.686 | 19.371 | 26.789 | 1.00 | 0.90 | 1SG | 2997 |
| ATOM | 2997 | O | LYS | 365 | 4.603 | 18.801 | 26.902 | 1.00 | 0.90 | 1SG | 2998 |
| ATOM | 2998 | N | TRP | 366 | 6.715 | 18.862 | 26.094 | 1.00 | 0.94 | 1SG | 2999 |
| ATOM | 2999 | CA | TRP | 366 | 6.696 | 17.651 | 25.328 | 1.00 | 0.94 | 1SG | 3000 |
| ATOM | 3000 | CB | TRP | 366 | 8.115 | 17.325 | 24.822 | 1.00 | 0.94 | 1SG | 3001 |
| ATOM | 3001 | CG | TRP | 366 | 9.061 | 17.102 | 25.982 | 1.00 | 0.94 | 1SG | 3002 |
| ATOM | 3002 | CD2 | TRP | 366 | 9.558 | 15.821 | 26.401 | 1.00 | 0.94 | 1SG | 3003 |
| ATOM | 3003 | CD1 | TRP | 366 | 9.592 | 18.018 | 26.840 | 1.00 | 0.94 | 1SG | 3004 |
| ATOM | 3004 | NE1 | TRP | 366 | 10.363 | 17.387 | 27.784 | 1.00 | 0.94 | 1SG | 3005 |
| ATOM | 3005 | CE2 | TRP | 366 | 10.360 | 16.035 | 27.523 | 1.00 | 0.94 | 1SG | 3006 |
| ATOM | 3006 | CE3 | TRP | 366 | 9.359 | 14.569 | 25.896 | 1.00 | 0.94 | 1SG | 3007 |
| ATOM | 3007 | CZ2 | TRP | 366 | 10.980 | 15.001 | 28.165 | 1.00 | 0.94 | 1SG | 3008 |
| ATOM | 3008 | CZ3 | TRP | 366 | 9.990 | 13.532 | 26.546 | 1.00 | 0.94 | 1SG | 3009 |
| ATOM | 3009 | CH2 | TRP | 366 | 10.782 | 13.738 | 27.656 | 1.00 | 0.94 | 1SG | 3010 |
| ATOM | 3010 | C | TRP | 366 | 6.234 | 16.510 | 26.190 | 1.00 | 0.94 | 1SG | 3011 |
| ATOM | 3011 | O | TRP | 366 | 6.816 | 16.207 | 27.231 | 1.00 | 0.94 | 1SG | 3012 |
| ATOM | 3012 | N | GLN | 367 | 5.152 | 15.844 | 25.744 | 1.00 | 0.65 | 1SG | 3013 |
| ATOM | 3013 | CA | GLN | 367 | 4.586 | 14.691 | 26.377 | 1.00 | 0.65 | 1SG | 3014 |
| ATOM | 3014 | CB | GLN | 367 | 5.417 | 13.415 | 26.186 | 1.00 | 0.65 | 1SG | 3015 |
| ATOM | 3015 | CG | GLN | 367 | 6.788 | 13.477 | 26.858 | 1.00 | 0.65 | 1SG | 3016 |
| ATOM | 3016 | CD | GLN | 367 | 7.419 | 12.108 | 26.707 | 1.00 | 0.65 | 1SG | 3017 |
| ATOM | 3017 | OE1 | GLN | 367 | 8.124 | 11.619 | 27.586 | 1.00 | 0.65 | 1SG | 3018 |
| ATOM | 3018 | NE2 | GLN | 367 | 7.172 | 11.467 | 25.540 | 1.00 | 0.65 | 1SG | 3019 |
| ATOM | 3019 | C | GLN | 367 | 4.448 | 14.913 | 27.848 | 1.00 | 0.65 | 1SG | 3020 |
| ATOM | 3020 | O | GLN | 367 | 4.567 | 13.976 | 28.636 | 1.00 | 0.65 | 1SG | 3021 |
| ATOM | 3021 | N | GLY | 368 | 4.167 | 16.158 | 28.266 | 1.00 | 0.45 | 1SG | 3022 |
| ATOM | 3022 | CA | GLY | 368 | 4.055 | 16.403 | 29.668 | 1.00 | 0.45 | 1SG | 3023 |
| ATOM | 3023 | C | GLY | 368 | 2.637 | 16.200 | 30.099 | 1.00 | 0.45 | 1SG | 3024 |
| ATOM | 3024 | O | GLY | 368 | 1.699 | 16.310 | 29.310 | 1.00 | 0.45 | 1SG | 3025 |
| ATOM | 3025 | N | HIS | 369 | 2.471 | 15.854 | 31.389 | 1.00 | 0.62 | 1SG | 3026 |
| ATOM | 3026 | CA | HIS | 369 | 1.199 | 15.784 | 32.050 | 1.00 | 0.62 | 1SG | 3027 |
| ATOM | 3027 | ND1 | HIS | 369 | −1.462 | 14.197 | 31.123 | 1.00 | 0.62 | 1SG | 3028 |
| ATOM | 3028 | CG | HIS | 369 | −0.199 | 13.732 | 31.412 | 1.00 | 0.62 | 1SG | 3029 |
| ATOM | 3029 | CB | HIS | 369 | 0.699 | 14.379 | 32.422 | 1.00 | 0.62 | 1SG | 3030 |
| ATOM | 3030 | NE2 | HIS | 369 | −1.178 | 12.349 | 29.924 | 1.00 | 0.62 | 1SG | 3031 |
| ATOM | 3031 | CD2 | HIS | 369 | −0.041 | 12.605 | 30.670 | 1.00 | 0.62 | 1SG | 3032 |
| ATOM | 3032 | CE1 | HIS | 369 | −2.003 | 13.333 | 30.228 | 1.00 | 0.62 | 1SG | 3033 |
| ATOM | 3033 | C | HIS | 369 | 1.426 | 16.453 | 33.359 | 1.00 | 0.62 | 1SG | 3034 |
| ATOM | 3034 | O | HIS | 369 | 2.449 | 16.209 | 33.996 | 1.00 | 0.62 | 1SG | 3035 |
| ATOM | 3035 | N | GLU | 370 | 0.495 | 17.326 | 33.797 | 1.00 | 0.74 | 1SG | 3036 |
| ATOM | 3036 | CA | GLU | 370 | 0.683 | 17.981 | 35.062 | 1.00 | 0.74 | 1SG | 3037 |
| ATOM | 3037 | CB | GLU | 370 | 0.994 | 16.964 | 36.187 | 1.00 | 0.74 | 1SG | 3038 |
| ATOM | 3038 | CG | GLU | 370 | 1.433 | 17.540 | 37.537 | 1.00 | 0.74 | 1SG | 3039 |
| ATOM | 3039 | CD | GLU | 370 | 0.223 | 18.103 | 38.261 | 1.00 | 0.74 | 1SG | 3040 |
| ATOM | 3040 | OE1 | GLU | 370 | −0.809 | 18.365 | 37.589 | 1.00 | 0.74 | 1SG | 3041 |
| ATOM | 3041 | OE2 | GLU | 370 | 0.320 | 18.281 | 39.504 | 1.00 | 0.74 | 1SG | 3042 |
| ATOM | 3042 | C | GLU | 370 | 1.828 | 18.934 | 34.930 | 1.00 | 0.74 | 1SG | 3043 |
| ATOM | 3043 | O | GLU | 370 | 2.243 | 19.572 | 35.896 | 1.00 | 0.74 | 1SG | 3044 |
| ATOM | 3044 | N | GLY | 371 | 2.353 | 19.082 | 33.703 | 1.00 | 0.46 | 1SG | 3045 |
| ATOM | 3045 | CA | GLY | 371 | 3.468 | 19.948 | 33.496 | 1.00 | 0.46 | 1SG | 3046 |
| ATOM | 3046 | C | GLY | 371 | 4.678 | 19.146 | 33.803 | 1.00 | 0.46 | 1SG | 3047 |
| ATOM | 3047 | O | GLY | 371 | 5.810 | 19.600 | 33.642 | 1.00 | 0.46 | 1SG | 3048 |
| ATOM | 3048 | N | ASP | 372 | 4.462 | 17.898 | 34.247 | 1.00 | 0.31 | 1SG | 3049 |
| ATOM | 3049 | CA | ASP | 372 | 5.593 | 17.109 | 34.599 | 1.00 | 0.31 | 1SG | 3050 |
| ATOM | 3050 | CB | ASP | 372 | 5.423 | 16.352 | 35.926 | 1.00 | 0.31 | 1SG | 3051 |
| ATOM | 3051 | CG | ASP | 372 | 5.402 | 17.383 | 37.046 | 1.00 | 0.31 | 1SG | 3052 |
| ATOM | 3052 | OD1 | ASP | 372 | 5.665 | 18.580 | 36.754 | 1.00 | 0.31 | 1SG | 3053 |
| ATOM | 3053 | OD2 | ASP | 372 | 5.115 | 16.990 | 38.208 | 1.00 | 0.31 | 1SG | 3054 |
| ATOM | 3054 | C | ASP | 372 | 5.809 | 16.109 | 33.527 | 1.00 | 0.31 | 1SG | 3055 |
| ATOM | 3055 | O | ASP | 372 | 4.893 | 15.406 | 33.104 | 1.00 | 0.31 | 1SG | 3056 |
| ATOM | 3056 | N | ILE | 373 | 7.041 | 16.070 | 32.998 | 1.00 | 0.49 | 1SG | 3057 |
| ATOM | 3057 | CA | ILE | 373 | 7.301 | 15.005 | 32.095 | 1.00 | 0.49 | 1SG | 3058 |
| ATOM | 3058 | CB | ILE | 373 | 8.421 | 15.288 | 31.139 | 1.00 | 0.49 | 1SG | 3059 |
| ATOM | 3059 | CG2 | ILE | 373 | 8.721 | 13.996 | 30.361 | 1.00 | 0.49 | 1SG | 3060 |
| ATOM | 3060 | CG1 | ILE | 373 | 8.053 | 16.475 | 30.231 | 1.00 | 0.49 | 1SG | 3061 |
| ATOM | 3061 | CD1 | ILE | 373 | 7.934 | 17.813 | 30.963 | 1.00 | 0.49 | 1SG | 3062 |
| ATOM | 3062 | C | ILE | 373 | 7.728 | 13.936 | 33.031 | 1.00 | 0.49 | 1SG | 3063 |
| ATOM | 3063 | O | ILE | 373 | 8.898 | 13.557 | 33.079 | 1.00 | 0.49 | 1SG | 3064 |
| ATOM | 3064 | N | ASP | 374 | 6.759 | 13.465 | 33.843 | 1.00 | 0.67 | 1SG | 3065 |
| ATOM | 3065 | CA | ASP | 374 | 7.028 | 12.452 | 34.814 | 1.00 | 0.67 | 1SG | 3066 |
| ATOM | 3066 | CB | ASP | 374 | 7.114 | 13.011 | 36.247 | 1.00 | 0.67 | 1SG | 3067 |
| ATOM | 3067 | CG | ASP | 374 | 7.746 | 11.932 | 37.106 | 1.00 | 0.67 | 1SG | 3068 |
| ATOM | 3068 | OD1 | ASP | 374 | 7.060 | 10.925 | 37.426 | 1.00 | 0.67 | 1SG | 3069 |
| ATOM | 3069 | OD2 | ASP | 374 | 8.946 | 12.106 | 37.438 | 1.00 | 0.67 | 1SG | 3070 |
| ATOM | 3070 | C | ASP | 374 | 5.871 | 11.509 | 34.778 | 1.00 | 0.67 | 1SG | 3071 |
| ATOM | 3071 | O | ASP | 374 | 5.987 | 10.343 | 35.155 | 1.00 | 0.67 | 1SG | 3072 |
| ATOM | 3072 | N | LYS | 375 | 4.709 | 12.000 | 34.306 | 1.00 | 0.63 | 1SG | 3073 |
| ATOM | 3073 | CA | LYS | 375 | 3.570 | 11.143 | 34.186 | 1.00 | 0.63 | 1SG | 3074 |
| ATOM | 3074 | CB | LYS | 375 | 2.224 | 11.886 | 34.257 | 1.00 | 0.63 | 1SG | 3075 |
| ATOM | 3075 | CG | LYS | 375 | 1.921 | 12.538 | 35.606 | 1.00 | 0.63 | 1SG | 3076 |
| ATOM | 3076 | CD | LYS | 375 | 0.720 | 13.488 | 35.569 | 1.00 | 0.63 | 1SG | 3077 |
| ATOM | 3077 | CE | LYS | 375 | −0.626 | 12.794 | 35.813 | 1.00 | 0.63 | 1SG | 3078 |
| ATOM | 3078 | NZ | LYS | 375 | −0.891 | 11.795 | 34.753 | 1.00 | 0.63 | 1SG | 3079 |
| ATOM | 3079 | C | LYS | 375 | 3.666 | 10.599 | 32.808 | 1.00 | 0.63 | 1SG | 3080 |
| ATOM | 3080 | O | LYS | 375 | 2.661 | 10.326 | 32.154 | 1.00 | 0.63 | 1SG | 3081 |
| ATOM | 3081 | N | GLY | 376 | 4.911 | 10.426 | 32.343 | 1.00 | 0.41 | 1SG | 3082 |
| ATOM | 3082 | CA | GLY | 376 | 5.170 | 9.953 | 31.024 | 1.00 | 0.41 | 1SG | 3083 |
| ATOM | 3083 | C | GLY | 376 | 6.541 | 10.432 | 30.729 | 1.00 | 0.41 | 1SG | 3084 |
| ATOM | 3084 | O | GLY | 376 | 6.903 | 10.661 | 29.580 | 1.00 | 0.41 | 1SG | 3085 |
| ATOM | 3085 | N | ALA | 377 | 7.339 | 10.572 | 31.802 | 1.00 | 0.32 | 1SG | 3086 |
| ATOM | 3086 | CA | ALA | 377 | 8.693 | 11.030 | 31.780 | 1.00 | 0.32 | 1SG | 3087 |
| ATOM | 3087 | CB | ALA | 377 | 9.409 | 10.865 | 33.130 | 1.00 | 0.32 | 1SG | 3088 |
| ATOM | 3088 | C | ALA | 377 | 9.463 | 10.198 | 30.818 | 1.00 | 0.32 | 1SG | 3089 |
| ATOM | 3089 | O | ALA | 377 | 8.961 | 9.291 | 30.160 | 1.00 | 0.32 | 1SG | 3090 |
| ATOM | 3090 | N | PRO | 378 | 10.698 | 10.568 | 30.710 | 1.00 | 0.61 | 1SG | 3091 |
| ATOM | 3091 | CA | PRO | 378 | 11.610 | 9.832 | 29.886 | 1.00 | 0.61 | 1SG | 3092 |
| ATOM | 3092 | CD | PRO | 378 | 11.041 | 11.979 | 30.774 | 1.00 | 0.61 | 1SG | 3093 |
| ATOM | 3093 | CB | PRO | 378 | 12.633 | 10.844 | 29.369 | 1.00 | 0.61 | 1SG | 3094 |
| ATOM | 3094 | CG | PRO | 378 | 12.504 | 12.043 | 30.320 | 1.00 | 0.61 | 1SG | 3095 |
| ATOM | 3095 | C | PRO | 378 | 12.233 | 8.771 | 30.727 | 1.00 | 0.61 | 1SG | 3096 |
| ATOM | 3096 | O | PRO | 378 | 11.995 | 8.749 | 31.934 | 1.00 | 0.61 | 1SG | 3097 |
| ATOM | 3097 | N | TYR | 379 | 13.028 | 7.382 | 30.107 | 1.00 | 0.95 | 1SG | 3098 |
| ATOM | 3098 | CA | TYR | 379 | 13.729 | 6.871 | 30.832 | 1.00 | 0.95 | 1SG | 3099 |
| ATOM | 3099 | CB | TYR | 379 | 13.263 | 5.427 | 30.570 | 1.00 | 0.95 | 1SG | 3100 |
| ATOM | 3100 | CG | TYR | 379 | 12.064 | 5.158 | 31.417 | 1.00 | 0.95 | 1SG | 3101 |
| ATOM | 3101 | CD1 | TYR | 379 | 12.247 | 4.733 | 32.718 | 1.00 | 0.95 | 1SG | 3102 |
| ATOM | 3102 | CD2 | TYR | 379 | 10.779 | 5.328 | 30.943 | 1.00 | 0.95 | 1SG | 3103 |
| ATOM | 3103 | CE1 | TYR | 379 | 11.172 | 4.473 | 33.535 | 1.00 | 0.95 | 1SG | 3104 |
| ATOM | 3104 | CE2 | TYR | 379 | 9.698 | 5.067 | 31.758 | 1.00 | 0.95 | 1SG | 3105 |
| ATOM | 3105 | CZ | TYR | 379 | 9.892 | 4.644 | 33.056 | 1.00 | 0.95 | 1SG | 3106 |
| ATOM | 3106 | OH | TYR | 379 | 8.785 | 4.380 | 33.890 | 1.00 | 0.95 | 1SG | 3107 |
| ATOM | 3107 | C | TYR | 379 | 15.159 | 6.939 | 30.434 | 1.00 | 0.95 | 1SG | 3108 |
| ATOM | 3108 | O | TYR | 379 | 15.666 | 8.002 | 30.080 | 1.00 | 0.95 | 1SG | 3109 |
| ATOM | 3109 | N | ALA | 380 | 15.828 | 5.775 | 30.546 | 1.00 | 1.06 | 1SG | 3110 |
| ATOM | 3110 | CA | ALA | 380 | 17.202 | 5.500 | 30.231 | 1.00 | 1.06 | 1SG | 3111 |
| ATOM | 3111 | CB | ALA | 380 | 17.399 | 4.933 | 28.827 | 1.00 | 1.06 | 1SG | 3112 |
| ATOM | 3112 | C | ALA | 380 | 18.059 | 6.712 | 30.317 | 1.00 | 1.06 | 1SG | 3113 |
| ATOM | 3113 | O | ALA | 380 | 18.337 | 7.374 | 29.319 | 1.00 | 1.06 | 1SG | 3114 |
| ATOM | 3114 | N | PRO | 381 | 18.472 | 7.026 | 31.503 | 1.00 | 1.12 | 1SG | 3115 |
| ATOM | 3115 | CA | PRO | 381 | 19.399 | 8.099 | 31.676 | 1.00 | 1.12 | 1SG | 3116 |
| ATOM | 3116 | CD | PRO | 381 | 17.605 | 6.884 | 32.647 | 1.00 | 1.12 | 1SG | 3117 |
| ATOM | 3117 | CB | PRO | 381 | 19.285 | 8.554 | 33.125 | 1.00 | 1.12 | 1SG | 3118 |
| ATOM | 3118 | CG | PRO | 381 | 17.867 | 8.122 | 33.521 | 1.00 | 1.12 | 1SG | 3119 |
| ATOM | 3119 | C | PRO | 381 | 20.718 | 7.501 | 31.363 | 1.00 | 1.12 | 1SG | 3120 |
| ATOM | 3120 | O | PRO | 381 | 21.381 | 7.983 | 30.448 | 1.00 | 1.12 | 1SG | 3121 |
| ATOM | 3121 | N | CYS | 382 | 21.112 | 6.450 | 32.113 | 1.00 | 1.08 | 1SG | 3122 |
| ATOM | 3122 | CA | CYS | 382 | 22.296 | 5.746 | 31.736 | 1.00 | 1.08 | 1SG | 3123 |
| ATOM | 3123 | CB | CYS | 382 | 22.622 | 4.533 | 32.625 | 1.00 | 1.08 | 1SG | 3124 |
| ATOM | 3124 | SG | CYS | 382 | 23.024 | 4.945 | 34.347 | 1.00 | 1.08 | 1SG | 3125 |
| ATOM | 3125 | C | CYS | 382 | 21.873 | 5.151 | 30.450 | 1.00 | 1.08 | 1SG | 3126 |
| ATOM | 3126 | O | CYS | 382 | 22.507 | 5.326 | 29.411 | 1.00 | 1.08 | 1SG | 3127 |
| ATOM | 3127 | N | SER | 383 | 20.723 | 4.453 | 30.545 | 1.00 | 0.95 | 1SG | 3128 |
| ATOM | 3128 | CA | SER | 383 | 20.048 | 3.804 | 29.469 | 1.00 | 0.95 | 1SG | 3129 |
| ATOM | 3129 | CB | SER | 383 | 20.944 | 3.377 | 28.291 | 1.00 | 0.95 | 1SG | 3130 |
| ATOM | 3130 | OG | SER | 383 | 20.159 | 2.749 | 27.287 | 1.00 | 0.95 | 1SG | 3131 |
| ATOM | 3131 | C | SER | 383 | 19.445 | 2.556 | 30.019 | 1.00 | 0.95 | 1SG | 3132 |
| ATOM | 3132 | O | SER | 383 | 20.114 | 1.777 | 30.695 | 1.00 | 0.95 | 1SG | 3133 |
| ATOM | 3133 | N | GLY | 384 | 18.125 | 2.394 | 29.801 | 1.00 | 0.78 | 1SG | 3134 |
| ATOM | 3134 | CA | GLY | 384 | 17.418 | 1.189 | 30.128 | 1.00 | 0.78 | 1SG | 3135 |
| ATOM | 3135 | C | GLY | 384 | 17.312 | 1.040 | 31.606 | 1.00 | 0.78 | 1SG | 3136 |
| ATOM | 3136 | O | GLY | 384 | 16.619 | 0.159 | 32.113 | 1.00 | 0.78 | 1SG | 3137 |
| ATOM | 3137 | N | ILE | 385 | 17.988 | 1.920 | 32.353 | 1.00 | 0.63 | 1SG | 3138 |
| ATOM | 3138 | CA | ILE | 385 | 17.940 | 1.772 | 33.767 | 1.00 | 0.63 | 1SG | 3139 |
| ATOM | 3139 | CB | ILE | 385 | 19.073 | 2.459 | 34.450 | 1.00 | 0.63 | 1SG | 3140 |
| ATOM | 3140 | CG2 | ILE | 385 | 20.376 | 1.765 | 34.023 | 1.00 | 0.63 | 1SG | 3141 |
| ATOM | 3141 | CG1 | ILE | 385 | 19.029 | 3.955 | 34.129 | 1.00 | 0.63 | 1SG | 3142 |
| ATOM | 3142 | CD1 | ILE | 385 | 20.020 | 4.766 | 34.953 | 1.00 | 0.63 | 1SG | 3143 |
| ATOM | 3143 | C | ILE | 385 | 16.657 | 2.362 | 34.216 | 1.00 | 0.63 | 1SG | 3144 |
| ATOM | 3144 | O | ILE | 385 | 16.032 | 3.142 | 33.500 | 1.00 | 0.63 | 1SG | 3145 |
| ATOM | 3145 | N | HIS | 386 | 16.213 | 1.967 | 35.417 | 1.00 | 0.53 | 1SG | 3146 |
| ATOM | 3146 | CA | HIS | 386 | 14.976 | 2.478 | 35.900 | 1.00 | 0.53 | 1SG | 3147 |
| ATOM | 3147 | ND1 | HIS | 386 | 16.124 | −0.108 | 37.675 | 1.00 | 0.53 | 1SG | 3148 |
| ATOM | 3148 | CG | HIS | 386 | 15.434 | 1.053 | 37.946 | 1.00 | 0.53 | 1SG | 3149 |
| ATOM | 3149 | CB | HIS | 386 | 14.388 | 1.623 | 37.034 | 1.00 | 0.53 | 1SG | 3150 |
| ATOM | 3150 | NE2 | HIS | 386 | 16.865 | 0.624 | 39.636 | 1.00 | 0.53 | 1SG | 3151 |
| ATOM | 3151 | CD2 | HIS | 386 | 15.900 | 1.489 | 39.147 | 1.00 | 0.53 | 1SG | 3152 |
| ATOM | 3152 | CE1 | HIS | 386 | 16.964 | −0.318 | 38.717 | 1.00 | 0.53 | 1SG | 3153 |
| ATOM | 3153 | C | HIS | 386 | 15.204 | 3.858 | 36.402 | 1.00 | 0.53 | 1SG | 3154 |
| ATOM | 3154 | O | HIS | 386 | 15.005 | 4.137 | 37.582 | 1.00 | 0.53 | 1SG | 3155 |
| ATOM | 3155 | N | GLN | 387 | 15.649 | 4.772 | 35.520 | 1.00 | 0.54 | 1SG | 3156 |
| ATOM | 3156 | CA | GLN | 387 | 15.739 | 6.105 | 36.017 | 1.00 | 0.54 | 1SG | 3157 |
| ATOM | 3157 | CB | GLN | 387 | 17.163 | 6.702 | 36.102 | 1.00 | 0.54 | 1SG | 3158 |
| ATOM | 3158 | CG | GLN | 387 | 18.117 | 5.936 | 37.033 | 1.00 | 0.54 | 1SG | 3159 |
| ATOM | 3159 | CD | GLN | 387 | 19.222 | 6.892 | 37.500 | 1.00 | 0.54 | 1SG | 3160 |
| ATOM | 3160 | OE1 | GLN | 387 | 19.006 | 7.647 | 38.447 | 1.00 | 0.54 | 1SG | 3161 |
| ATOM | 3161 | NE2 | GLN | 387 | 20.416 | 6.879 | 36.846 | 1.00 | 0.54 | 1SG | 3162 |
| ATOM | 3162 | C | GLN | 387 | 14.906 | 6.957 | 35.121 | 1.00 | 0.54 | 1SG | 3163 |
| ATOM | 3163 | O | GLN | 387 | 15.108 | 7.026 | 33.912 | 1.00 | 0.54 | 1SG | 3164 |
| ATOM | 3164 | N | ARG | 388 | 13.891 | 7.609 | 35.701 | 1.00 | 0.61 | 1SG | 3165 |
| ATOM | 3165 | CA | ARG | 388 | 13.086 | 8.499 | 34.926 | 1.00 | 0.61 | 1SG | 3166 |
| ATOM | 3166 | CB | ARG | 388 | 11.696 | 7.944 | 34.556 | 1.00 | 0.61 | 1SG | 3167 |
| ATOM | 3167 | CG | ARG | 388 | 10.785 | 7.647 | 35.749 | 1.00 | 0.61 | 1SG | 3168 |
| ATOM | 3168 | CD | ARG | 388 | 10.117 | 8.883 | 36.359 | 1.00 | 0.61 | 1SG | 3169 |
| ATOM | 3169 | NE | ARG | 388 | 9.216 | 8.393 | 37.436 | 1.00 | 0.61 | 1SG | 3170 |
| ATOM | 3170 | CZ | ARG | 388 | 7.972 | 7.947 | 37.108 | 1.00 | 0.61 | 1SG | 3171 |
| ATOM | 3171 | NH1 | ARG | 388 | 7.540 | 8.017 | 35.814 | 1.00 | 0.61 | 1SG | 3172 |
| ATOM | 3172 | NH2 | ARG | 388 | 7.172 | 7.408 | 38.071 | 1.00 | 0.61 | 1SG | 3173 |
| ATOM | 3173 | C | ARG | 388 | 12.886 | 9.691 | 35.793 | 1.00 | 0.61 | 1SG | 3174 |
| ATOM | 3174 | O | ARG | 388 | 12.874 | 9.567 | 37.017 | 1.00 | 0.61 | 1SG | 3175 |
| ATOM | 3175 | N | ALA | 389 | 12.761 | 10.892 | 35.199 | 1.00 | 0.51 | 1SG | 3176 |
| ATOM | 3176 | CA | ALA | 389 | 12.540 | 11.989 | 36.092 | 1.00 | 0.51 | 1SG | 3177 |
| ATOM | 3177 | CB | ALA | 389 | 13.730 | 12.244 | 37.030 | 1.00 | 0.51 | 1SG | 3178 |
| ATOM | 3178 | C | ALA | 389 | 12.322 | 13.253 | 35.324 | 1.00 | 0.51 | 1SG | 3179 |
| ATOM | 3179 | O | ALA | 389 | 12.738 | 13.357 | 34.172 | 1.00 | 0.51 | 1SG | 3180 |
| ATOM | 3180 | N | ILE | 390 | 11.580 | 14.199 | 35.953 | 1.00 | 0.45 | 1SG | 3181 |
| ATOM | 3181 | CA | ILE | 390 | 11.370 | 15.569 | 35.556 | 1.00 | 0.45 | 1SG | 3182 |
| ATOM | 3182 | CB | ILE | 390 | 11.643 | 15.887 | 34.117 | 1.00 | 0.45 | 1SG | 3183 |
| ATOM | 3183 | CG2 | ILE | 390 | 10.846 | 17.142 | 33.724 | 1.00 | 0.45 | 1SG | 3184 |
| ATOM | 3184 | CG1 | ILE | 390 | 13.160 | 16.045 | 33.937 | 1.00 | 0.45 | 1SG | 3185 |
| ATOM | 3185 | CD1 | ILE | 390 | 13.600 | 16.015 | 32.485 | 1.00 | 0.45 | 1SG | 3186 |
| ATOM | 3186 | C | ILE | 390 | 9.995 | 16.000 | 35.945 | 1.00 | 0.45 | 1SG | 3187 |
| ATOM | 3187 | O | ILE | 390 | 9.001 | 15.470 | 35.449 | 1.00 | 0.45 | 1SG | 3188 |
| ATOM | 3188 | N | CYS | 391 | 9.916 | 17.014 | 36.833 | 1.00 | 0.54 | 1SG | 3189 |
| ATOM | 3189 | CA | CYS | 391 | 8.647 | 17.497 | 37.294 | 1.00 | 0.54 | 1SG | 3190 |
| ATOM | 3190 | CB | CYS | 391 | 8.144 | 16.778 | 38.555 | 1.00 | 0.54 | 1SG | 3191 |
| ATOM | 3191 | SG | CYS | 391 | 9.209 | 17.100 | 39.992 | 1.00 | 0.54 | 1SG | 3192 |
| ATOM | 3192 | C | CYS | 391 | 8.809 | 18.937 | 37.658 | 1.00 | 0.54 | 1SG | 3193 |
| ATOM | 3193 | O | CYS | 391 | 9.902 | 19.495 | 37.580 | 1.00 | 0.54 | 1SG | 3194 |
| ATOM | 3194 | N | VAL | 392 | 7.697 | 19.582 | 38.058 | 1.00 | 0.59 | 1SG | 3195 |
| ATOM | 3195 | CA | VAL | 392 | 7.737 | 20.963 | 38.436 | 1.00 | 0.59 | 1SG | 3196 |
| ATOM | 3196 | CB | VAL | 392 | 6.907 | 21.848 | 37.556 | 1.00 | 0.59 | 1SG | 3197 |
| ATOM | 3197 | CG1 | VAL | 392 | 7.473 | 21.784 | 36.127 | 1.00 | 0.59 | 1SG | 3198 |
| ATOM | 3198 | CG2 | VAL | 392 | 5.437 | 21.410 | 37.665 | 1.00 | 0.59 | 1SG | 3199 |
| ATOM | 3199 | C | VAL | 392 | 7.166 | 21.052 | 39.810 | 1.00 | 0.59 | 1SG | 3200 |
| ATOM | 3200 | O | VAL | 392 | 6.753 | 20.048 | 40.387 | 1.00 | 0.59 | 1SG | 3201 |
| ATOM | 3201 | N | TYR | 393 | 7.149 | 22.272 | 40.378 | 1.00 | 0.60 | 1SG | 3202 |
| ATOM | 3202 | CA | TYR | 393 | 6.630 | 22.456 | 41.699 | 1.00 | 0.60 | 1SG | 3203 |
| ATOM | 3203 | CB | TYR | 393 | 6.836 | 23.876 | 42.260 | 1.00 | 0.60 | 1SG | 3204 |
| ATOM | 3204 | CG | TYR | 393 | 6.126 | 24.866 | 41.404 | 1.00 | 0.60 | 1SG | 3205 |
| ATOM | 3205 | CD1 | TYR | 393 | 6.714 | 25.338 | 40.254 | 1.00 | 0.60 | 1SG | 3206 |
| ATOM | 3206 | CD2 | TYR | 393 | 4.881 | 25.331 | 41.759 | 1.00 | 0.60 | 1SG | 3207 |
| ATOM | 3207 | CE1 | TYR | 393 | 6.067 | 26.256 | 39.462 | 1.00 | 0.60 | 1SG | 3208 |
| ATOM | 3208 | CE2 | TYR | 393 | 4.230 | 26.250 | 40.970 | 1.00 | 0.60 | 1SG | 3209 |
| ATOM | 3209 | CZ | TYR | 393 | 4.823 | 26.713 | 39.819 | 1.00 | 0.60 | 1SG | 3210 |
| ATOM | 3210 | OH | TYR | 393 | 4.159 | 27.656 | 39.007 | 1.00 | 0.60 | 1SG | 3211 |
| ATOM | 3211 | C | TYR | 393 | 5.171 | 22.145 | 41.679 | 1.00 | 0.60 | 1SG | 3212 |
| ATOM | 3212 | O | TYR | 393 | 4.610 | 21.744 | 42.697 | 1.00 | 0.60 | 1SG | 3213 |
| ATOM | 3213 | N | GLY | 394 | 4.505 | 22.293 | 40.516 | 1.00 | 0.60 | 1SG | 3214 |
| ATOM | 3214 | CA | GLY | 394 | 3.110 | 21.964 | 40.515 | 1.00 | 0.60 | 1SG | 3215 |
| ATOM | 3215 | C | GLY | 394 | 2.333 | 22.935 | 39.676 | 1.00 | 0.60 | 1SG | 3216 |
| ATOM | 3216 | O | GLY | 394 | 1.318 | 22.554 | 39.097 | 1.00 | 0.60 | 1SG | 3217 |
| ATOM | 3217 | N | ALA | 395 | 2.757 | 24.212 | 39.583 | 1.00 | 0.71 | 1SG | 3218 |
| ATOM | 3218 | CA | ALA | 395 | 1.972 | 25.126 | 38.793 | 1.00 | 0.71 | 1SG | 3219 |
| ATOM | 3219 | CB | ALA | 395 | 1.688 | 26.467 | 39.491 | 1.00 | 0.71 | 1SG | 3220 |
| ATOM | 3220 | C | ALA | 395 | 2.695 | 25.423 | 37.512 | 1.00 | 0.71 | 1SG | 3221 |
| ATOM | 3221 | O | ALA | 395 | 3.802 | 24.936 | 37.287 | 1.00 | 0.71 | 1SG | 3222 |
| ATOM | 3222 | N | GLY | 396 | 2.065 | 26.231 | 36.626 | 1.00 | 0.84 | 1SG | 3223 |
| ATOM | 3223 | CA | GLY | 396 | 2.675 | 26.569 | 35.365 | 1.00 | 0.84 | 1SG | 3224 |
| ATOM | 3224 | C | GLY | 396 | 1.674 | 27.323 | 34.534 | 1.00 | 0.84 | 1SG | 3225 |
| ATOM | 3225 | O | GLY | 396 | 0.509 | 27.423 | 34.914 | 1.00 | 0.84 | 1SG | 3226 |
| ATOM | 3226 | N | ASP | 397 | 2.101 | 27.878 | 33.368 | 1.00 | 0.84 | 1SG | 3227 |
| ATOM | 3227 | CA | ASP | 397 | 1.161 | 28.629 | 32.573 | 1.00 | 0.84 | 1SG | 3228 |
| ATOM | 3228 | CB | ASP | 397 | 0.726 | 29.955 | 33.231 | 1.00 | 0.84 | 1SG | 3229 |
| ATOM | 3229 | CG | ASP | 397 | 1.937 | 30.863 | 33.400 | 1.00 | 0.84 | 1SG | 3230 |
| ATOM | 3230 | OD1 | ASP | 397 | 3.074 | 30.403 | 33.115 | 1.00 | 0.84 | 1SG | 3231 |
| ATOM | 3231 | OD2 | ASP | 397 | 1.738 | 32.032 | 33.826 | 1.00 | 0.84 | 1SG | 3232 |
| ATOM | 3232 | C | ASP | 397 | 1.724 | 28.952 | 31.217 | 1.00 | 0.84 | 1SG | 3233 |
| ATOM | 3233 | O | ASP | 397 | 2.707 | 28.369 | 30.759 | 1.00 | 0.84 | 1SG | 3234 |
| ATOM | 3234 | N | LEU | 398 | 1.054 | 29.914 | 30.540 | 1.00 | 0.66 | 1SG | 3235 |
| ATOM | 3235 | CA | LEU | 398 | 1.311 | 30.421 | 29.215 | 1.00 | 0.66 | 1SG | 3236 |
| ATOM | 3236 | CB | LEU | 398 | 0.251 | 31.444 | 28.761 | 1.00 | 0.66 | 1SG | 3237 |
| ATOM | 3237 | CG | LEU | 398 | 0.261 | 32.769 | 29.555 | 1.00 | 0.66 | 1SG | 3238 |
| ATOM | 3238 | CD2 | LEU | 398 | 0.167 | 32.538 | 31.071 | 1.00 | 0.66 | 1SG | 3239 |
| ATOM | 3239 | CD1 | LEU | 398 | −0.824 | 33.729 | 29.043 | 1.00 | 0.66 | 1SG | 3240 |
| ATOM | 3240 | C | LEU | 398 | 2.624 | 31.101 | 29.156 | 1.00 | 0.66 | 1SG | 3241 |
| ATOM | 3241 | O | LEU | 398 | 3.320 | 31.020 | 28.145 | 1.00 | 0.66 | 1SG | 3242 |
| ATOM | 3242 | N | ASN | 399 | 2.990 | 31.770 | 30.256 | 1.00 | 0.42 | 1SG | 3243 |
| ATOM | 3243 | CA | ASN | 399 | 4.197 | 32.537 | 30.295 | 1.00 | 0.42 | 1SG | 3244 |
| ATOM | 3244 | CB | ASN | 399 | 4.503 | 33.117 | 31.686 | 1.00 | 0.42 | 1SG | 3245 |
| ATOM | 3245 | CG | ASN | 399 | 3.500 | 34.225 | 31.973 | 1.00 | 0.42 | 1SG | 3246 |
| ATOM | 3246 | OD1 | ASN | 399 | 3.472 | 34.784 | 33.068 | 1.00 | 0.42 | 1SG | 3247 |
| ATOM | 3247 | ND2 | ASN | 399 | 2.651 | 34.552 | 30.962 | 1.00 | 0.42 | 1SG | 3248 |
| ATOM | 3248 | C | ASN | 399 | 5.301 | 31.612 | 29.940 | 1.00 | 0.42 | 1SG | 3249 |
| ATOM | 3249 | O | ASN | 399 | 6.294 | 32.026 | 29.345 | 1.00 | 0.42 | 1SG | 3250 |
| ATOM | 3250 | N | TRP | 400 | 5.144 | 30.328 | 30.303 | 1.00 | 0.48 | 1SG | 3251 |
| ATOM | 3251 | CA | TRP | 400 | 6.140 | 29.358 | 29.982 | 1.00 | 0.48 | 1SG | 3252 |
| ATOM | 3252 | CB | TRP | 400 | 5.794 | 27.971 | 30.551 | 1.00 | 0.48 | 1SG | 3253 |
| ATOM | 3253 | CG | TRP | 400 | 5.842 | 27.917 | 32.060 | 1.00 | 0.48 | 1SG | 3254 |
| ATOM | 3254 | CD2 | TRP | 400 | 6.494 | 26.886 | 32.817 | 1.00 | 0.48 | 1SG | 3255 |
| ATOM | 3255 | CD1 | TRP | 400 | 5.256 | 28.749 | 32.968 | 1.00 | 0.48 | 1SG | 3256 |
| ATOM | 3256 | NE1 | TRP | 400 | 5.518 | 28.314 | 34.245 | 1.00 | 0.48 | 1SG | 3257 |
| ATOM | 3257 | CE2 | TRP | 400 | 6.274 | 27.163 | 34.166 | 1.00 | 0.48 | 1SG | 3258 |
| ATOM | 3258 | CE3 | TRP | 400 | 7.212 | 25.796 | 32.421 | 1.00 | 0.48 | 1SG | 3259 |
| ATOM | 3259 | CZ2 | TRP | 400 | 6.774 | 26.350 | 35.143 | 1.00 | 0.48 | 1SG | 3260 |
| ATOM | 3260 | CZ3 | TRP | 400 | 7.713 | 24.976 | 33.407 | 1.00 | 0.48 | 1SG | 3261 |
| ATOM | 3261 | CH2 | TRP | 400 | 7.499 | 25.250 | 34.742 | 1.00 | 0.48 | 1SG | 3262 |
| ATOM | 3262 | C | TRP | 400 | 6.186 | 29.266 | 28.495 | 1.00 | 0.48 | 1SG | 3263 |
| ATOM | 3263 | O | TRP | 400 | 5.401 | 28.552 | 27.871 | 1.00 | 0.48 | 1SG | 3264 |
| ATOM | 3264 | N | MET | 401 | 7.125 | 30.019 | 27.889 | 1.00 | 0.87 | 1SG | 3265 |
| ATOM | 3265 | CA | MET | 401 | 7.275 | 30.038 | 26.470 | 1.00 | 0.87 | 1SG | 3266 |
| ATOM | 3266 | CB | MET | 401 | 7.144 | 31.450 | 25.873 | 1.00 | 0.87 | 1SG | 3267 |
| ATOM | 3267 | CG | MET | 401 | 7.296 | 31.506 | 24.352 | 1.00 | 0.87 | 1SG | 3268 |
| ATOM | 3268 | SD | MET | 401 | 7.137 | 33.175 | 23.648 | 1.00 | 0.87 | 1SG | 3269 |
| ATOM | 3269 | CE | MET | 401 | 5.352 | 33.308 | 23.958 | 1.00 | 0.87 | 1SG | 3270 |
| ATOM | 3270 | C | MET | 401 | 8.664 | 29.584 | 26.198 | 1.00 | 0.87 | 1SG | 3271 |
| ATOM | 3271 | O | MET | 401 | 9.611 | 30.061 | 26.820 | 1.00 | 0.87 | 1SG | 3272 |
| ATOM | 3272 | N | LEU | 402 | 8.826 | 28.622 | 25.277 | 1.00 | 1.22 | 1SG | 3273 |
| ATOM | 3273 | CA | LEU | 402 | 10.161 | 28.209 | 24.982 | 1.00 | 1.22 | 1SG | 3274 |
| ATOM | 3274 | CB | LEU | 402 | 10.352 | 26.681 | 25.040 | 1.00 | 1.22 | 1SG | 3275 |
| ATOM | 3275 | CG | LEU | 402 | 11.808 | 26.199 | 24.855 | 1.00 | 1.22 | 1SG | 3276 |
| ATOM | 3276 | CD2 | LEU | 402 | 12.722 | 26.797 | 25.937 | 1.00 | 1.22 | 1SG | 3277 |
| ATOM | 3277 | CD1 | LEU | 402 | 12.337 | 26.438 | 23.432 | 1.00 | 1.22 | 1SG | 3278 |
| ATOM | 3278 | C | LEU | 402 | 10.421 | 28.667 | 23.589 | 1.00 | 1.22 | 1SG | 3279 |
| ATOM | 3279 | O | LEU | 402 | 10.182 | 27.937 | 22.628 | 1.00 | 1.22 | 1SG | 3280 |
| ATOM | 3280 | N | GLN | 403 | 10.903 | 29.914 | 23.444 | 1.00 | 1.25 | 1SG | 3281 |
| ATOM | 3281 | CA | GLN | 403 | 11.186 | 30.404 | 22.132 | 1.00 | 1.25 | 1SG | 3282 |
| ATOM | 3282 | CB | GLN | 403 | 10.430 | 31.699 | 21.781 | 1.00 | 1.25 | 1SG | 3283 |
| ATOM | 3283 | CG | GLN | 403 | 10.646 | 32.188 | 20.345 | 1.00 | 1.25 | 1SG | 3284 |
| ATOM | 3284 | CD | GLN | 403 | 11.984 | 32.908 | 20.256 | 1.00 | 1.25 | 1SG | 3285 |
| ATOM | 3285 | OE1 | GLN | 403 | 12.557 | 33.319 | 21.264 | 1.00 | 1.25 | 1SG | 3286 |
| ATOM | 3286 | NE2 | GLN | 403 | 12.499 | 33.074 | 19.008 | 1.00 | 1.25 | 1SG | 3287 |
| ATOM | 3287 | C | GLN | 403 | 12.644 | 30.700 | 22.095 | 1.00 | 1.25 | 1SG | 3288 |
| ATOM | 3288 | O | GLN | 403 | 13.153 | 31.474 | 22.905 | 1.00 | 1.25 | 1SG | 3289 |
| ATOM | 3289 | N | ASN | 404 | 13.365 | 30.058 | 21.160 | 1.00 | 1.18 | 1SG | 3290 |
| ATOM | 3290 | CA | ASN | 404 | 14.766 | 30.319 | 21.048 | 1.00 | 1.18 | 1SG | 3291 |
| ATOM | 3291 | CB | ASN | 404 | 15.651 | 29.118 | 21.428 | 1.00 | 1.18 | 1SG | 3292 |
| ATOM | 3292 | CG | ASN | 404 | 15.347 | 27.979 | 20.465 | 1.00 | 1.18 | 1SG | 3293 |
| ATOM | 3293 | OD1 | ASN | 404 | 14.187 | 27.658 | 20.212 | 1.00 | 1.18 | 1SG | 3294 |
| ATOM | 3294 | ND2 | ASN | 404 | 16.417 | 27.355 | 19.905 | 1.00 | 1.18 | 1SG | 3295 |
| ATOM | 3295 | C | ASN | 404 | 15.028 | 30.635 | 19.616 | 1.00 | 1.18 | 1SG | 3296 |
| ATOM | 3296 | O | ASN | 404 | 14.553 | 29.942 | 18.718 | 1.00 | 1.18 | 1SG | 3297 |
| ATOM | 3297 | N | HIS | 405 | 15.777 | 31.723 | 19.363 | 1.00 | 1.25 | 1SG | 3298 |
| ATOM | 3298 | CA | HIS | 405 | 16.081 | 32.050 | 18.006 | 1.00 | 1.25 | 1SG | 3299 |
| ATOM | 3299 | ND1 | HIS | 405 | 17.333 | 35.227 | 18.178 | 1.00 | 1.25 | 1SG | 3300 |
| ATOM | 3300 | CG | HIS | 405 | 16.159 | 34.558 | 18.442 | 1.00 | 1.25 | 1SG | 3301 |
| ATOM | 3301 | CB | HIS | 405 | 15.606 | 33.455 | 17.589 | 1.00 | 1.25 | 1SG | 3302 |
| ATOM | 3302 | NE2 | HIS | 405 | 16.507 | 36.109 | 20.044 | 1.00 | 1.25 | 1SG | 3303 |
| ATOM | 3303 | CD2 | HIS | 405 | 15.668 | 35.109 | 19.585 | 1.00 | 1.25 | 1SG | 3304 |
| ATOM | 3304 | CE1 | HIS | 405 | 17.493 | 36.143 | 19.167 | 1.00 | 1.25 | 1SG | 3305 |
| ATOM | 3305 | C | HIS | 405 | 17.563 | 31.999 | 17.865 | 1.00 | 1.25 | 1SG | 3306 |
| ATOM | 3306 | O | HIS | 405 | 18.289 | 32.762 | 18.501 | 1.00 | 1.25 | 1SG | 3307 |
| ATOM | 3307 | N | HIS | 406 | 18.059 | 31.061 | 17.039 | 1.00 | 1.34 | 1SG | 3308 |
| ATOM | 3308 | CA | HIS | 406 | 19.469 | 30.979 | 16.833 | 1.00 | 1.34 | 1SG | 3309 |
| ATOM | 3309 | ND1 | HIS | 406 | 20.138 | 27.798 | 15.998 | 1.00 | 1.34 | 1SG | 3310 |
| ATOM | 3310 | CG | HIS | 406 | 19.640 | 28.464 | 17.096 | 1.00 | 1.34 | 1SG | 3311 |
| ATOM | 3311 | CB | HIS | 406 | 20.136 | 29.801 | 17.563 | 1.00 | 1.34 | 1SG | 3312 |
| ATOM | 3312 | NE2 | HIS | 406 | 18.540 | 26.508 | 16.852 | 1.00 | 1.34 | 1SG | 3313 |
| ATOM | 3313 | CD2 | HIS | 406 | 18.665 | 27.662 | 17.605 | 1.00 | 1.34 | 1SG | 3314 |
| ATOM | 3314 | CE1 | HIS | 406 | 19.445 | 26.635 | 15.898 | 1.00 | 1.34 | 1SG | 3315 |
| ATOM | 3315 | C | HIS | 406 | 19.681 | 30.773 | 15.376 | 1.00 | 1.34 | 1SG | 3316 |
| ATOM | 3316 | O | HIS | 406 | 19.016 | 29.947 | 14.750 | 1.00 | 1.34 | 1SG | 3317 |
| ATOM | 3317 | N | LEU | 407 | 20.607 | 31.545 | 14.784 | 1.00 | 1.28 | 1SG | 3318 |
| ATOM | 3318 | CA | LEU | 407 | 20.876 | 31.378 | 13.388 | 1.00 | 1.28 | 1SG | 3319 |
| ATOM | 3319 | CB | LEU | 407 | 20.642 | 32.649 | 12.554 | 1.00 | 1.28 | 1SG | 3320 |
| ATOM | 3320 | CG | LEU | 407 | 20.930 | 32.459 | 11.052 | 1.00 | 1.28 | 1SG | 3321 |
| ATOM | 3321 | CD2 | LEU | 407 | 20.939 | 33.806 | 10.314 | 1.00 | 1.28 | 1SG | 3322 |
| ATOM | 3322 | CD1 | LEU | 407 | 19.969 | 31.440 | 10.426 | 1.00 | 1.28 | 1SG | 3323 |
| ATOM | 3323 | C | LEU | 407 | 22.316 | 31.025 | 13.267 | 1.00 | 1.28 | 1SG | 3324 |
| ATOM | 3324 | O | LEU | 407 | 23.176 | 31.685 | 13.848 | 1.00 | 1.28 | 1SG | 3325 |
| ATOM | 3325 | N | LEU | 408 | 22.619 | 29.948 | 12.523 | 1.00 | 0.98 | 1SG | 3326 |
| ATOM | 3326 | CA | LEU | 408 | 23.988 | 29.580 | 12.355 | 1.00 | 0.98 | 1SG | 3327 |
| ATOM | 3327 | CB | LEU | 408 | 24.238 | 28.067 | 12.481 | 1.00 | 0.98 | 1SG | 3328 |
| ATOM | 3328 | CG | LEU | 408 | 23.926 | 27.507 | 13.882 | 1.00 | 0.98 | 1SG | 3329 |
| ATOM | 3329 | CD2 | LEU | 408 | 24.436 | 26.067 | 14.035 | 1.00 | 0.98 | 1SG | 3330 |
| ATOM | 3330 | CD1 | LEU | 408 | 22.433 | 27.650 | 14.219 | 1.00 | 0.98 | 1SG | 3331 |
| ATOM | 3331 | C | LEU | 408 | 24.374 | 29.979 | 10.973 | 1.00 | 0.98 | 1SG | 3332 |
| ATOM | 3332 | O | LEU | 408 | 24.006 | 29.323 | 10.000 | 1.00 | 0.98 | 1SG | 3333 |
| ATOM | 3333 | N | ALA | 409 | 25.118 | 31.092 | 10.852 | 1.00 | 0.75 | 1SG | 3334 |
| ATOM | 3334 | CA | ALA | 409 | 25.528 | 31.520 | 9.551 | 1.00 | 0.75 | 1SG | 3335 |
| ATOM | 3335 | CB | ALA | 409 | 25.446 | 33.044 | 9.357 | 1.00 | 0.75 | 1SG | 3336 |
| ATOM | 3336 | C | ALA | 409 | 26.957 | 31.131 | 9.400 | 1.00 | 0.75 | 1SG | 3337 |
| ATOM | 3337 | O | ALA | 409 | 27.838 | 31.985 | 9.305 | 1.00 | 0.75 | 1SG | 3338 |
| ATOM | 3338 | N | ASN | 410 | 27.227 | 29.814 | 9.375 | 1.00 | 0.94 | 1SG | 3339 |
| ATOM | 3339 | CA | ASN | 410 | 28.580 | 29.382 | 9.219 | 1.00 | 0.94 | 1SG | 3340 |
| ATOM | 3340 | CB | ASN | 410 | 28.978 | 28.245 | 10.179 | 1.00 | 0.94 | 1SG | 3341 |
| ATOM | 3341 | CG | ASN | 410 | 30.478 | 28.013 | 10.055 | 1.00 | 0.94 | 1SG | 3342 |
| ATOM | 3342 | OD1 | ASN | 410 | 31.017 | 27.886 | 8.956 | 1.00 | 0.94 | 1SG | 3343 |
| ATOM | 3343 | ND2 | ASN | 410 | 31.179 | 27.960 | 11.219 | 1.00 | 0.94 | 1SG | 3344 |
| ATOM | 3344 | C | ASN | 410 | 28.712 | 28.860 | 7.831 | 1.00 | 0.94 | 1SG | 3345 |
| ATOM | 3345 | O | ASN | 410 | 28.247 | 27.765 | 7.521 | 1.00 | 0.94 | 1SG | 3346 |
| ATOM | 3346 | N | LYS | 411 | 29.343 | 29.652 | 6.946 | 1.00 | 1.25 | 1SG | 3347 |
| ATOM | 3347 | CA | LYS | 411 | 29.526 | 29.201 | 5.602 | 1.00 | 1.25 | 1SG | 3348 |
| ATOM | 3348 | CB | LYS | 411 | 29.075 | 30.218 | 4.539 | 1.00 | 1.25 | 1SG | 3349 |
| ATOM | 3349 | CG | LYS | 411 | 29.273 | 29.730 | 3.103 | 1.00 | 1.25 | 1SG | 3350 |
| ATOM | 3350 | CD | LYS | 411 | 28.365 | 28.557 | 2.728 | 1.00 | 1.25 | 1SG | 3351 |
| ATOM | 3351 | CE | LYS | 411 | 26.892 | 28.942 | 2.570 | 1.00 | 1.25 | 1SG | 3352 |
| ATOM | 3352 | NZ | LYS | 411 | 26.707 | 29.735 | 1.334 | 1.00 | 1.25 | 1SG | 3353 |
| ATOM | 3353 | C | LYS | 411 | 30.990 | 28.993 | 5.424 | 1.00 | 1.25 | 1SG | 3354 |
| ATOM | 3354 | O | LYS | 411 | 31.779 | 29.929 | 5.541 | 1.00 | 1.25 | 1SG | 3355 |
| ATOM | 3355 | N | PHE | 412 | 31.397 | 27.741 | 5.153 | 1.00 | 1.19 | 1SG | 3356 |
| ATOM | 3356 | CA | PHE | 412 | 32.792 | 27.490 | 4.966 | 1.00 | 1.19 | 1SG | 3357 |
| ATOM | 3357 | CB | PHE | 412 | 33.366 | 26.442 | 5.936 | 1.00 | 1.19 | 1SG | 3358 |
| ATOM | 3358 | CG | PHE | 412 | 32.663 | 25.154 | 5.680 | 1.00 | 1.19 | 1SG | 3359 |
| ATOM | 3359 | CD1 | PHE | 412 | 31.442 | 24.897 | 6.261 | 1.00 | 1.19 | 1SG | 3360 |
| ATOM | 3360 | CD2 | PHE | 412 | 33.224 | 24.201 | 4.861 | 1.00 | 1.19 | 1SG | 3361 |
| ATOM | 3361 | CE1 | PHE | 412 | 30.790 | 23.709 | 6.027 | 1.00 | 1.19 | 1SG | 3362 |
| ATOM | 3362 | CE2 | PHE | 412 | 32.576 | 23.011 | 4.624 | 1.00 | 1.19 | 1SG | 3363 |
| ATOM | 3363 | CZ | PHE | 412 | 31.357 | 22.764 | 5.207 | 1.00 | 1.19 | 1SG | 3364 |
| ATOM | 3364 | C | PHE | 412 | 32.968 | 26.965 | 3.583 | 1.00 | 1.19 | 1SG | 3365 |
| ATOM | 3365 | O | PHE | 412 | 32.225 | 26.092 | 3.136 | 1.00 | 1.19 | 1SG | 3366 |
| ATOM | 3366 | N | ASP | 413 | 33.955 | 27.516 | 2.855 | 1.00 | 1.00 | 1SG | 3367 |
| ATOM | 3367 | CA | ASP | 413 | 34.215 | 27.045 | 1.531 | 1.00 | 1.00 | 1SG | 3368 |
| ATOM | 3368 | CB | ASP | 413 | 33.860 | 28.057 | 0.426 | 1.00 | 1.00 | 1SG | 3369 |
| ATOM | 3369 | CG | ASP | 413 | 34.715 | 29.305 | 0.606 | 1.00 | 1.00 | 1SG | 3370 |
| ATOM | 3370 | OD1 | ASP | 413 | 35.210 | 29.532 | 1.742 | 1.00 | 1.00 | 1SG | 3371 |
| ATOM | 3371 | OD2 | ASP | 413 | 34.880 | 30.053 | −0.394 | 1.00 | 1.00 | 1SG | 3372 |
| ATOM | 3372 | C | ASP | 413 | 35.680 | 26.782 | 1.450 | 1.00 | 1.00 | 1SG | 3373 |
| ATOM | 3373 | O | ASP | 413 | 36.478 | 27.374 | 2.175 | 1.00 | 1.00 | 1SG | 3374 |
| ATOM | 3374 | N | PRO | 414 | 36.046 | 25.872 | 0.598 | 1.00 | 0.95 | 1SG | 3375 |
| ATOM | 3375 | CA | PRO | 414 | 37.447 | 25.602 | 0.457 | 1.00 | 0.95 | 1SG | 3376 |
| ATOM | 3376 | CD | PRO | 414 | 35.245 | 24.668 | 0.436 | 1.00 | 0.95 | 1SG | 3377 |
| ATOM | 3377 | CB | PRO | 414 | 37.551 | 24.216 | −0.173 | 1.00 | 0.95 | 1SG | 3378 |
| ATOM | 3378 | CG | PRO | 414 | 36.254 | 23.519 | 0.273 | 1.00 | 0.95 | 1SG | 3379 |
| ATOM | 3379 | C | PRO | 414 | 38.058 | 26.684 | −0.364 | 1.00 | 0.95 | 1SG | 3380 |
| ATOM | 3380 | O | PRO | 414 | 37.367 | 27.249 | −1.211 | 1.00 | 0.95 | 1SG | 3381 |
| ATOM | 3381 | N | LYS | 415 | 39.341 | 27.005 | −0.123 | 1.00 | 0.74 | 1SG | 3382 |
| ATOM | 3382 | CA | LYS | 415 | 39.965 | 28.025 | −0.908 | 1.00 | 0.74 | 1SG | 3383 |
| ATOM | 3383 | CB | LYS | 415 | 40.572 | 29.163 | −0.069 | 1.00 | 0.74 | 1SG | 3384 |
| ATOM | 3384 | CG | LYS | 415 | 39.526 | 29.992 | 0.680 | 1.00 | 0.74 | 1SG | 3385 |
| ATOM | 3385 | CD | LYS | 415 | 40.123 | 30.909 | 1.749 | 1.00 | 0.74 | 1SG | 3386 |
| ATOM | 3386 | CE | LYS | 415 | 40.855 | 32.124 | 1.174 | 1.00 | 0.74 | 1SG | 3387 |
| ATOM | 3387 | NZ | LYS | 415 | 39.878 | 33.091 | 0.627 | 1.00 | 0.74 | 1SG | 3388 |
| ATOM | 3388 | C | LYS | 415 | 41.085 | 27.375 | −1.645 | 1.00 | 0.74 | 1SG | 3389 |
| ATOM | 3389 | O | LYS | 415 | 42.076 | 26.958 | −1.047 | 1.00 | 0.74 | 1SG | 3390 |
| ATOM | 3390 | N | VAL | 416 | 40.945 | 27.255 | −2.977 | 1.00 | 0.63 | 1SG | 3391 |
| ATOM | 3391 | CA | VAL | 416 | 41.996 | 26.657 | −3.739 | 1.00 | 0.63 | 1SG | 3392 |
| ATOM | 3392 | CB | VAL | 416 | 41.559 | 25.457 | −4.527 | 1.00 | 0.63 | 1SG | 3393 |
| ATOM | 3393 | CG1 | VAL | 416 | 42.753 | 24.955 | −5.356 | 1.00 | 0.63 | 1SG | 3394 |
| ATOM | 3394 | CG2 | VAL | 416 | 40.992 | 24.411 | −3.552 | 1.00 | 0.63 | 1SG | 3395 |
| ATOM | 3395 | C | VAL | 416 | 42.459 | 27.680 | −4.717 | 1.00 | 0.63 | 1SG | 3396 |
| ATOM | 3396 | O | VAL | 416 | 41.658 | 28.268 | −5.443 | 1.00 | 0.63 | 1SG | 3397 |
| ATOM | 3397 | N | ASP | 417 | 43.779 | 27.935 | −4.745 | 1.00 | 0.87 | 1SG | 3398 |
| ATOM | 3398 | CA | ASP | 417 | 44.292 | 28.887 | −5.681 | 1.00 | 0.87 | 1SG | 3399 |
| ATOM | 3399 | CB | ASP | 417 | 44.975 | 30.101 | −5.025 | 1.00 | 0.87 | 1SG | 3400 |
| ATOM | 3400 | CG | ASP | 417 | 46.170 | 29.617 | −4.215 | 1.00 | 0.87 | 1SG | 3401 |
| ATOM | 3401 | OD1 | ASP | 417 | 46.089 | 28.499 | −3.641 | 1.00 | 0.87 | 1SG | 3402 |
| ATOM | 3402 | OD2 | ASP | 417 | 47.185 | 30.363 | −4.163 | 1.00 | 0.87 | 1SG | 3403 |
| ATOM | 3403 | C | ASP | 417 | 45.313 | 28.182 | −6.506 | 1.00 | 0.87 | 1SG | 3404 |
| ATOM | 3404 | O | ASP | 417 | 46.200 | 27.512 | −5.979 | 1.00 | 0.87 | 1SG | 3405 |
| ATOM | 3405 | N | ASP | 418 | 45.196 | 28.296 | −7.841 | 1.00 | 1.07 | 1SG | 3406 |
| ATOM | 3406 | CA | ASP | 418 | 46.158 | 27.658 | −8.684 | 1.00 | 1.07 | 1SG | 3407 |
| ATOM | 3407 | CB | ASP | 418 | 45.559 | 26.587 | −9.613 | 1.00 | 1.07 | 1SG | 3408 |
| ATOM | 3408 | CG | ASP | 418 | 44.563 | 27.255 | −10.551 | 1.00 | 1.07 | 1SG | 3409 |
| ATOM | 3409 | OD1 | ASP | 418 | 43.874 | 28.211 | −10.104 | 1.00 | 1.07 | 1SG | 3410 |
| ATOM | 3410 | OD2 | ASP | 418 | 44.474 | 26.813 | −11.727 | 1.00 | 1 07 | 1SG | 3411 |
| ATOM | 3411 | C | ASP | 418 | 46.757 | 28.717 | −9.545 | 1.00 | 1.07 | 1SG | 3412 |
| ATOM | 3412 | O | ASP | 418 | 46.048 | 29.528 | −10.138 | 1.00 | 1.07 | 1SG | 3413 |
| ATOM | 3413 | N | ASN | 419 | 48.099 | 28.750 | −9.615 | 1.00 | 0.99 | 1SG | 3414 |
| ATOM | 3414 | CA | ASN | 419 | 48.738 | 29.722 | −10.445 | 1.00 | 0.99 | 1SG | 3415 |
| ATOM | 3415 | CB | ASN | 419 | 49.641 | 30.700 | −9.674 | 1.00 | 0.99 | 1SG | 3416 |
| ATOM | 3416 | CG | ASN | 419 | 50.762 | 29.901 | −9.022 | 1.00 | 0.99 | 1SG | 3417 |
| ATOM | 3417 | OD1 | ASN | 419 | 50.525 | 28.861 | −8.409 | 1.00 | 0.99 | 1SG | 3418 |
| ATOM | 3418 | ND2 | ASN | 419 | 52.021 | 30.396 | −9.163 | 1.00 | 0.99 | 1SG | 3419 |
| ATOM | 3419 | C | ASN | 419 | 49.600 | 28.973 | −11.403 | 1.00 | 0.99 | 1SG | 3420 |
| ATOM | 3420 | O | ASN | 419 | 50.332 | 28.063 | −11.016 | 1.00 | 0.99 | 1SG | 3421 |
| ATOM | 3421 | N | ALA | 420 | 49.513 | 29.324 | −12.698 | 1.00 | 0.96 | 1SG | 3422 |
| ATOM | 3422 | CA | ALA | 420 | 50.335 | 28.657 | −13.660 | 1.00 | 0.96 | 1SG | 3423 |
| ATOM | 3423 | CB | ALA | 420 | 49.553 | 28.159 | −14.890 | 1.00 | 0.96 | 1SG | 3424 |
| ATOM | 3424 | C | ALA | 420 | 51.331 | 29.650 | −14.140 | 1.00 | 0.96 | 1SG | 3425 |
| ATOM | 3425 | O | ALA | 420 | 51.052 | 30.438 | −15.046 | 1.00 | 0.96 | 1SG | 3426 |
| ATOM | 3426 | N | LEU | 421 | 52.528 | 29.654 | −13.532 | 1.00 | 1.10 | 1SG | 3427 |
| ATOM | 3427 | CA | LEU | 421 | 53.532 | 30.576 | −13.962 | 1.00 | 1.10 | 1SG | 3428 |
| ATOM | 3428 | CB | LEU | 421 | 54.083 | 31.461 | −12.832 | 1.00 | 1.10 | 1SG | 3429 |
| ATOM | 3429 | CG | LEU | 421 | 55.158 | 32.460 | −13.300 | 1.00 | 1.10 | 1SG | 3430 |
| ATOM | 3430 | CD2 | LEU | 421 | 55.859 | 33.126 | −12.104 | 1.00 | 1.10 | 1SG | 3431 |
| ATOM | 3431 | CD1 | LEU | 421 | 54.586 | 33.473 | −14.297 | 1.00 | 1.10 | 1SG | 3432 |
| ATOM | 3432 | C | LEU | 421 | 54.671 | 29.775 | −14.490 | 1.00 | 1.10 | 1SG | 3433 |
| ATOM | 3433 | O | LEU | 421 | 55.245 | 28.951 | −13.779 | 1.00 | 1.10 | 1SG | 3434 |
| ATOM | 3434 | N | GLN | 422 | 55.013 | 29.986 | −15.772 | 1.00 | 1.00 | 1SG | 3435 |
| ATOM | 3435 | CA | GLN | 422 | 56.117 | 29.272 | −16.334 | 1.00 | 1.00 | 1SG | 3436 |
| ATOM | 3436 | CB | GLN | 422 | 55.738 | 28.390 | −17.536 | 1.00 | 1.00 | 1SG | 3437 |
| ATOM | 3437 | CG | GLN | 422 | 56.922 | 27.628 | −18.130 | 1.00 | 1.00 | 1SG | 3438 |
| ATOM | 3438 | CD | GLN | 422 | 56.410 | 26.804 | −19.303 | 1.00 | 1.00 | 1SG | 3439 |
| ATOM | 3439 | OE1 | GLN | 422 | 57.169 | 26.091 | −19.957 | 1.00 | 1.00 | 1SG | 3440 |
| ATOM | 3440 | NE2 | GLN | 422 | 55.080 | 26.895 | −19.575 | 1.00 | 1.00 | 1SG | 3441 |
| ATOM | 3441 | C | GLN | 422 | 57.083 | 30.296 | −16.821 | 1.00 | 1.00 | 1SG | 3442 |
| ATOM | 3442 | O | GLN | 422 | 56.710 | 31.220 | −17.541 | 1.00 | 1.00 | 1SG | 3443 |
| ATOM | 3443 | N | CYS | 423 | 58.362 | 30.164 | −16.419 | 1.00 | 0.94 | 1SG | 3444 |
| ATOM | 3444 | CA | CYS | 423 | 59.335 | 31.108 | −16.872 | 1.00 | 0.94 | 1SG | 3445 |
| ATOM | 3445 | CB | CYS | 423 | 60.111 | 31.790 | −15.729 | 1.00 | 0.94 | 1SG | 3446 |
| ATOM | 3446 | SG | CYS | 423 | 61.335 | 32.986 | −16.341 | 1.00 | 0.94 | 1SG | 3447 |
| ATOM | 3447 | C | CYS | 423 | 60.324 | 30.355 | −17.692 | 1.00 | 0.94 | 1SG | 3448 |
| ATOM | 3448 | O | CYS | 423 | 60.885 | 29.356 | −17.241 | 1.00 | 0.94 | 1SG | 3449 |
| ATOM | 3449 | N | LEU | 424 | 60.548 | 30.808 | −18.937 | 1.00 | 1.21 | 1SG | 3450 |
| ATOM | 3450 | CA | LEU | 424 | 61.505 | 30.142 | −19.766 | 1.00 | 1.21 | 1SG | 3451 |
| ATOM | 3451 | CB | LEU | 424 | 60.921 | 29.619 | −21.085 | 1.00 | 1.21 | 1SG | 3452 |
| ATOM | 3452 | CG | LEU | 424 | 61.956 | 28.907 | −21.978 | 1.00 | 1.21 | 1SG | 3453 |
| ATOM | 3453 | CD2 | LEU | 424 | 61.395 | 28.635 | −23.383 | 1.00 | 1.21 | 1SG | 3454 |
| ATOM | 3454 | CD1 | LEU | 424 | 62.494 | 27.638 | −21.297 | 1.00 | 1.21 | 1SG | 3455 |
| ATOM | 3455 | C | LEU | 424 | 62.546 | 31.152 | −20.111 | 1.00 | 1.21 | 1SG | 3456 |
| ATOM | 3456 | O | LEU | 424 | 62.234 | 32.247 | −20.578 | 1.00 | 1.21 | 1SG | 3457 |
| ATOM | 3457 | N | GLU | 425 | 63.824 | 30.814 | −19.868 | 1.00 | 1.33 | 1SG | 3458 |
| ATOM | 3458 | CA | GLU | 425 | 64.869 | 31.733 | −20.196 | 1.00 | 1.33 | 1SG | 3459 |
| ATOM | 3459 | CB | GLU | 425 | 65.675 | 32.212 | −18.972 | 1.00 | 1.33 | 1SG | 3460 |
| ATOM | 3460 | CG | GLU | 425 | 66.378 | 31.087 | −18.204 | 1.00 | 1.33 | 1SG | 3461 |
| ATOM | 3461 | CD | GLU | 425 | 67.711 | 30.796 | −18.879 | 1.00 | 1.33 | 1SG | 3462 |
| ATOM | 3462 | OE1 | GLU | 425 | 68.120 | 31.598 | −19.761 | 1.00 | 1.33 | 1SG | 3463 |
| ATOM | 3463 | OE2 | GLU | 425 | 68.342 | 29.767 | −18.517 | 1.00 | 1.33 | 1SG | 3464 |
| ATOM | 3464 | C | GLU | 425 | 65.800 | 31.022 | −21.115 | 1.00 | 1.33 | 1SG | 3465 |
| ATOM | 3465 | O | GLU | 425 | 66.122 | 29.854 | −20.911 | 1.00 | 1.33 | 1SG | 3466 |
| ATOM | 3466 | N | GLU | 426 | 66.241 | 31.717 | −22.181 | 1.00 | 1.23 | 1SG | 3467 |
| ATOM | 3467 | CA | GLU | 426 | 67.163 | 31.107 | −23.088 | 1.00 | 1.23 | 1SG | 3468 |
| ATOM | 3468 | CB | GLU | 426 | 66.665 | 31.075 | −24.542 | 1.00 | 1.23 | 1SG | 3469 |
| ATOM | 3469 | CG | GLU | 426 | 67.651 | 30.415 | −25.510 | 1.00 | 1.23 | 1SG | 3470 |
| ATOM | 3470 | CD | GLU | 426 | 67.039 | 30.452 | −26.904 | 1.00 | 1.23 | 1SG | 3471 |
| ATOM | 3471 | OE1 | GLU | 426 | 65.900 | 30.973 | −27.038 | 1.00 | 1.23 | 1SG | 3472 |
| ATOM | 3472 | OE2 | GLU | 426 | 67.699 | 29.949 | −27.852 | 1.00 | 1.23 | 1SG | 3473 |
| ATOM | 3473 | C | GLU | 426 | 68.400 | 31.933 | −23.061 | 1.00 | 1.23 | 1SG | 3474 |
| ATOM | 3474 | O | GLU | 426 | 68.348 | 33.153 | −23.187 | 1.00 | 1.23 | 1SG | 3475 |
| ATOM | 3475 | N | TYR | 427 | 69.564 | 31.278 | −22.871 | 1.00 | 1.13 | 1SG | 3476 |
| ATOM | 3476 | CA | TYR | 427 | 70.785 | 32.013 | −22.851 | 1.00 | 1.13 | 1SG | 3477 |
| ATOM | 3477 | CB | TYR | 427 | 71.594 | 31.829 | −21.555 | 1.00 | 1.13 | 1SG | 3478 |
| ATOM | 3478 | CG | TYR | 427 | 72.836 | 32.646 | −21.666 | 1.00 | 1.13 | 1SG | 3479 |
| ATOM | 3479 | CD1 | TYR | 427 | 73.951 | 32.149 | −22.299 | 1.00 | 1.13 | 1SG | 3480 |
| ATOM | 3480 | CD2 | TYR | 427 | 72.883 | 33.915 | −21.134 | 1.00 | 1.13 | 1SG | 3481 |
| ATOM | 3481 | CE1 | TYR | 427 | 75.097 | 32.905 | −22.399 | 1.00 | 1.13 | 1SG | 3482 |
| ATOM | 3482 | CE2 | TYR | 427 | 74.023 | 34.674 | −21.231 | 1.00 | 1.13 | 1SG | 3483 |
| ATOM | 3483 | CZ | TYR | 427 | 75.132 | 34.170 | −21.864 | 1.00 | 1.13 | 1SG | 3484 |
| ATOM | 3484 | OH | TYR | 427 | 76.306 | 34.946 | −21.965 | 1.00 | 1.13 | 1SG | 3485 |
| ATOM | 3485 | C | TYR | 427 | 71.617 | 31.500 | −23.979 | 1.00 | 1.13 | 1SG | 3486 |
| ATOM | 3486 | O | TYR | 427 | 71.699 | 30.295 | −24.199 | 1.00 | 1.13 | 1SG | 3487 |
| ATOM | 3487 | N | LEU | 428 | 72.234 | 32.420 | −24.740 | 1.00 | 1.11 | 1SG | 3488 |
| ATOM | 3488 | CA | LEU | 428 | 73.060 | 31.993 | −25.828 | 1.00 | 1.11 | 1SG | 3489 |
| ATOM | 3489 | CB | LEU | 428 | 72.627 | 32.558 | −27.191 | 1.00 | 1.11 | 1SG | 3490 |
| ATOM | 3490 | CG | LEU | 428 | 71.236 | 32.075 | −27.641 | 1.00 | 1.11 | 1SG | 3491 |
| ATOM | 3491 | CD2 | LEU | 428 | 70.963 | 32.447 | −29.107 | 1.00 | 1.11 | 1SG | 3492 |
| ATOM | 3492 | CD1 | LEU | 428 | 70.137 | 32.570 | −26.687 | 1.00 | 1.11 | 1SG | 3493 |
| ATOM | 3493 | C | LEU | 428 | 74.434 | 32.503 | −25.553 | 1.00 | 1.11 | 1SG | 3494 |
| ATOM | 3494 | O | LEU | 428 | 74.606 | 33.594 | −25.011 | 1.00 | 1.11 | 1SG | 3495 |
| ATOM | 3495 | N | ARG | 429 | 75.456 | 31.705 | −25.907 | 1.00 | 1.23 | 1SG | 3496 |
| ATOM | 3496 | CA | ARG | 429 | 76.802 | 32.142 | −25.689 | 1.00 | 1.23 | 1SG | 3497 |
| ATOM | 3497 | CB | ARG | 429 | 77.692 | 31.081 | −25.017 | 1.00 | 1.23 | 1SG | 3498 |
| ATOM | 3498 | CG | ARG | 429 | 77.285 | 30.760 | −23.579 | 1.00 | 1.23 | 1SG | 3499 |
| ATOM | 3499 | CD | ARG | 429 | 77.814 | 31.762 | −22.550 | 1.00 | 1.23 | 1SG | 3500 |
| ATOM | 3500 | NE | ARG | 429 | 79.300 | 31.664 | −22.547 | 1.00 | 1.23 | 1SG | 3501 |
| ATOM | 3501 | CZ | ARG | 429 | 80.037 | 32.411 | −23.420 | 1.00 | 1.23 | 1SG | 3502 |
| ATOM | 3502 | NH1 | ARG | 429 | 79.415 | 33.258 | −24.291 | 1.00 | 1.23 | 1SG | 3503 |
| ATOM | 3503 | NH2 | ARG | 429 | 81.399 | 32.313 | −23.420 | 1.00 | 1.23 | 1SG | 3504 |
| ATOM | 3504 | C | ARG | 429 | 77.385 | 32.421 | −27.030 | 1.00 | 1.23 | 1SG | 3505 |
| ATOM | 3505 | O | ARG | 429 | 77.200 | 31.656 | −27.973 | 1.00 | 1.23 | 1SG | 3506 |
| ATOM | 3506 | N | TYR | 430 | 78.092 | 33.558 | −27.153 | 1.00 | 1.38 | 1SG | 3507 |
| ATOM | 3507 | CA | TYR | 430 | 78.699 | 33.884 | −28.405 | 1.00 | 1.38 | 1SG | 3508 |
| ATOM | 3508 | CB | TYR | 430 | 78.366 | 35.301 | −28.898 | 1.00 | 1.38 | 1SG | 3509 |
| ATOM | 3509 | CG | TYR | 430 | 79.049 | 35.510 | −30.206 | 1.00 | 1.38 | 1SG | 3510 |
| ATOM | 3510 | CD1 | TYR | 430 | 80.360 | 35.928 | −30.248 | 1.00 | 1.38 | 1SG | 3511 |
| ATOM | 3511 | CD2 | TYR | 430 | 78.383 | 35.289 | −31.389 | 1.00 | 1.38 | 1SG | 3512 |
| ATOM | 3512 | CE1 | TYR | 430 | 80.996 | 36.122 | −31.453 | 1.00 | 1.38 | 1SG | 3513 |
| ATOM | 3513 | CE2 | TYR | 430 | 79.013 | 35.479 | −32.594 | 1.00 | 1.38 | 1SG | 3514 |
| ATOM | 3514 | CZ | TYR | 430 | 80.320 | 35.896 | −32.629 | 1.00 | 1.38 | 1SG | 3515 |
| ATOM | 3515 | OH | TYR | 430 | 80.970 | 36.095 | −33.867 | 1.00 | 1.38 | 1SG | 3516 |
| ATOM | 3516 | C | TYR | 430 | 80.172 | 33.815 | −28.197 | 1.00 | 1.38 | 1SG | 3517 |
| ATOM | 3517 | O | TYR | 430 | 80.695 | 34.327 | −27.210 | 1.00 | 1.38 | 1SG | 3518 |
| ATOM | 3518 | N | LYS | 431 | 80.884 | 33.152 | −29.126 | 1.00 | 1.22 | 1SG | 3519 |
| ATOM | 3519 | CA | LYS | 431 | 82.305 | 33.059 | −28.986 | 1.00 | 1.22 | 1SG | 3520 |
| ATOM | 3520 | CB | LYS | 431 | 82.840 | 31.618 | −29.059 | 1.00 | 1.22 | 1SG | 3521 |
| ATOM | 3521 | CG | LYS | 431 | 84.360 | 31.524 | −28.904 | 1.00 | 1.22 | 1SG | 3522 |
| ATOM | 3522 | CD | LYS | 431 | 84.857 | 31.909 | −27.509 | 1.00 | 1.22 | 1SG | 3523 |
| ATOM | 3523 | CE | LYS | 431 | 86.376 | 31.814 | −27.353 | 1.00 | 1.22 | 1SG | 3524 |
| ATOM | 3524 | NZ | LYS | 431 | 86.806 | 30.402 | −27.440 | 1.00 | 1.22 | 1SG | 3525 |
| ATOM | 3525 | C | LYS | 431 | 82.904 | 33.811 | −30.125 | 1.00 | 1.22 | 1SG | 3526 |
| ATOM | 3526 | O | LYS | 431 | 82.400 | 33.770 | −31.246 | 1.00 | 1.22 | 1SG | 3527 |
| ATOM | 3527 | N | ALA | 432 | 83.997 | 34.545 | −29.853 | 1.00 | 0.92 | 1SG | 3528 |
| ATOM | 3528 | CA | ALA | 432 | 84.635 | 35.283 | −30.897 | 1.00 | 0.92 | 1SG | 3529 |
| ATOM | 3529 | CB | ALA | 432 | 85.383 | 36.534 | −30.404 | 1.00 | 0.92 | 1SG | 3530 |
| ATOM | 3530 | C | ALA | 432 | 85.642 | 34.374 | −31.517 | 1.00 | 0.92 | 1SG | 3531 |
| ATOM | 3531 | O | ALA | 432 | 85.877 | 33.269 | −31.031 | 1.00 | 0.92 | 1SG | 3532 |
| ATOM | 3532 | N | ILE | 433 | 86.250 | 34.818 | −32.631 | 1.00 | 0.82 | 1SG | 3533 |
| ATOM | 3533 | CA | ILE | 433 | 87.238 | 34.003 | −33.267 | 1.00 | 0.82 | 1SG | 3534 |
| ATOM | 3534 | CB | ILE | 433 | 87.057 | 33.889 | −34.754 | 1.00 | 0.82 | 1SG | 3535 |
| ATOM | 3535 | CG2 | ILE | 433 | 87.269 | 35.283 | −35.364 | 1.00 | 0.82 | 1SG | 3536 |
| ATOM | 3536 | CG1 | ILE | 433 | 87.988 | 32.807 | −35.326 | 1.00 | 0.82 | 1SG | 3537 |
| ATOM | 3537 | CD1 | ILE | 433 | 87.639 | 31.394 | −34.861 | 1.00 | 0.82 | 1SG | 3538 |
| ATOM | 3538 | C | ILE | 433 | 88.557 | 34.649 | −33.028 | 1.00 | 0.82 | 1SG | 3539 |
| ATOM | 3539 | O | ILE | 433 | 88.683 | 35.874 | −33.085 | 1.00 | 0.82 | 1SG | 3540 |
| ATOM | 3540 | N | TYR | 434 | 89.583 | 33.834 | −32.726 | 1.00 | 0.64 | 1SG | 3541 |
| ATOM | 3541 | CA | TYR | 434 | 90.883 | 34.381 | −32.493 | 1.00 | 0.64 | 1SG | 3542 |
| ATOM | 3542 | CB | TYR | 434 | 91.465 | 34.022 | −31.115 | 1.00 | 0.64 | 1SG | 3543 |
| ATOM | 3543 | CG | TYR | 434 | 92.810 | 34.656 | −31.013 | 1.00 | 0.64 | 1SG | 3544 |
| ATOM | 3544 | CD1 | TYR | 434 | 92.933 | 35.980 | −30.655 | 1.00 | 0.64 | 1SG | 3545 |
| ATOM | 3545 | CD2 | TYR | 434 | 93.946 | 33.928 | −31.274 | 1.00 | 0.64 | 1SG | 3546 |
| ATOM | 3546 | CE1 | TYR | 434 | 94.173 | 36.566 | −30.560 | 1.00 | 0.64 | 1SG | 3547 |
| ATOM | 3547 | CE2 | TYR | 434 | 95.190 | 34.509 | −31.181 | 1.00 | 0.64 | 1SG | 3548 |
| ATOM | 3548 | CZ | TYR | 434 | 95.303 | 35.831 | −30.824 | 1.00 | 0.64 | 1SG | 3549 |
| ATOM | 3549 | OH | TYR | 434 | 96.577 | 36.431 | −30.726 | 1.00 | 0.64 | 1SG | 3550 |
| ATOM | 3550 | C | TYR | 434 | 91.788 | 33.797 | −33.524 | 1.00 | 0.64 | 1SG | 3551 |
| ATOM | 3551 | O | TYR | 434 | 91.516 | 32.726 | −34.069 | 1.00 | 0.64 | 1SG | 3552 |
| ATOM | 3552 | N | GLY | 435 | 92.886 | 34.505 | −33.835 | 1.00 | 0.55 | 1SG | 3553 |
| ATOM | 3553 | CA | GLY | 435 | 93.804 | 33.999 | −34.809 | 1.00 | 0.55 | 1SG | 3554 |
| ATOM | 3554 | C | GLY | 435 | 94.811 | 35.068 | −35.061 | 1.00 | 0.55 | 1SG | 3555 |
| ATOM | 3555 | O | GLY | 435 | 94.495 | 36.256 | −35.036 | 1.00 | 0.55 | 1SG | 3556 |
| ATOM | 3556 | N | THR | 436 | 96.067 | 34.659 | −35.315 | 1.00 | 0.92 | 1SG | 3557 |
| ATOM | 3557 | CA | THR | 436 | 97.095 | 35.617 | −35.578 | 1.00 | 0.92 | 1SG | 3558 |
| ATOM | 3558 | CB | THR | 436 | 98.311 | 35.448 | −34.715 | 1.00 | 0.92 | 1SG | 3559 |
| ATOM | 3559 | OG1 | THR | 436 | 99.202 | 36.537 | −34.902 | 1.00 | 0.92 | 1SG | 3560 |
| ATOM | 3560 | CG2 | THR | 436 | 99.000 | 34.125 | −35.087 | 1.00 | 0.92 | 1SG | 3561 |
| ATOM | 3561 | C | THR | 436 | 97.526 | 35.420 | −36.991 | 1.00 | 0.92 | 1SG | 3562 |
| ATOM | 3562 | O | THR | 436 | 97.240 | 34.390 | −37.599 | 1.00 | 0.92 | 1SG | 3563 |
| ATOM | 3563 | N | GLU | 437 | 98.211 | 36.427 | −37.559 | 1.00 | 1.23 | 1SG | 3564 |
| ATOM | 3564 | CA | GLU | 437 | 98.663 | 36.306 | −38.909 | 1.00 | 1.23 | 1SG | 3565 |
| ATOM | 3565 | CB | GLU | 437 | 99.176 | 37.631 | −39.503 | 1.00 | 1.23 | 1SG | 3566 |
| ATOM | 3566 | CG | GLU | 437 | 99.495 | 37.561 | −40.998 | 1.00 | 1.23 | 1SG | 3567 |
| ATOM | 3567 | CD | GLU | 437 | 98.177 | 37.494 | −41.757 | 1.00 | 1.23 | 1SG | 3568 |
| ATOM | 3568 | OE1 | GLU | 437 | 97.108 | 37.506 | −41.091 | 1.00 | 1.23 | 1SG | 3569 |
| ATOM | 3569 | OE2 | GLU | 437 | 98.222 | 37.431 | −43.015 | 1.00 | 1.23 | 1SG | 3570 |
| ATOM | 3570 | C | GLU | 437 | 99.791 | 35.329 | −38.898 | 1.00 | 1.23 | 1SG | 3571 |
| ATOM | 3571 | O | GLU | 437 | 100.175 | 34.823 | −37.846 | 1.00 | 1.23 | 1SG | 3572 |
| ATOM | 3572 | N | LEU | 438 | 100.341 | 35.024 | −40.087 | 1.00 | 1.30 | 1SG | 3573 |
| ATOM | 3573 | CA | LEU | 438 | 101.430 | 34.100 | −40.152 | 1.00 | 1.30 | 1SG | 3574 |
| ATOM | 3574 | CB | LEU | 438 | 101.968 | 33.920 | −41.588 | 1.00 | 1.30 | 1SG | 3575 |
| ATOM | 3575 | CG | LEU | 438 | 103.019 | 32.805 | −41.801 | 1.00 | 1.30 | 1SG | 3576 |
| ATOM | 3576 | CD2 | LEU | 438 | 104.232 | 32.923 | −40.854 | 1.00 | 1.30 | 1SG | 3577 |
| ATOM | 3577 | CD1 | LEU | 438 | 103.461 | 32.753 | −43.273 | 1.00 | 1.30 | 1SG | 3578 |
| ATOM | 3578 | C | LEU | 438 | 102.515 | 34.712 | −39.281 | 1.00 | 1.30 | 1SG | 3579 |
| ATOM | 3579 | O | LEU | 438 | 102.913 | 34.055 | −38.283 | 1.00 | 1.30 | 1SG | 3580 |
| ATOM | 3580 | OXT | LEU | 438 | 102.961 | 35.843 | −39.605 | 1.00 | 1.30 | 1SG | 3581 |
| END | | | | | | | | | | | |

TABLE 7
Core 2c human model

| | | | | | | | | | | | |
|---|---|---|---|---|---|---|---|---|---|---|---|
| ATOM | 1 | N | LEU | 1 | −23.456 | −7.859 | 11.219 | 1.00 | 0.43 | 1SG | 2 |
| ATOM | 2 | CA | LEU | 1 | −23.436 | −6.655 | 12.080 | 1.00 | 0.43 | 1SG | 3 |
| ATOM | 3 | CB | LEU | 1 | −23.337 | −7.041 | 13.568 | 1.00 | 0.43 | 1SG | 4 |
| ATOM | 4 | CG | LEU | 1 | −24.604 | −7.722 | 14.126 | 1.00 | 0.43 | 1SG | 5 |
| ATOM | 5 | CD2 | LEU | 1 | −24.895 | −9.064 | 13.433 | 1.00 | 0.43 | 1SG | 6 |
| ATOM | 6 | CD1 | LEU | 1 | −25.802 | −6.759 | 14.100 | 1.00 | 0.43 | 1SG | 7 |
| ATOM | 7 | C | LEU | 1 | −22.274 | −5.794 | 11.729 | 1.00 | 0.43 | 1SG | 8 |
| ATOM | 8 | O | LEU | 1 | −21.548 | −6.069 | 10.774 | 1.00 | 0.43 | 1SG | 9 |
| ATOM | 9 | N | VAL | 2 | −22.077 | −4.705 | 12.491 | 1.00 | 0.63 | 1SG | 10 |
| ATOM | 10 | CA | VAL | 2 | −20.990 | −3.831 | 12.186 | 1.00 | 0.63 | 1SG | 11 |
| ATOM | 11 | CB | VAL | 2 | −21.290 | −2.387 | 12.463 | 1.00 | 0.63 | 1SG | 12 |
| ATOM | 12 | CG1 | VAL | 2 | −20.033 | −1.561 | 12.145 | 1.00 | 0.63 | 1SG | 13 |
| ATOM | 13 | CG2 | VAL | 2 | −22.534 | −1.973 | 11.658 | 1.00 | 0.63 | 1SG | 14 |
| ATOM | 14 | C | VAL | 2 | −19.854 | −4.208 | 13.072 | 1.00 | 0.63 | 1SG | 15 |
| ATOM | 15 | O | VAL | 2 | −19.891 | −3.972 | 14.278 | 1.00 | 0.63 | 1SG | 16 |
| ATOM | 16 | N | GLU | 3 | −18.806 | −4.821 | 12.494 | 1.00 | 0.96 | 1SG | 17 |
| ATOM | 17 | CA | GLU | 3 | −17.680 | −5.135 | 13.314 | 1.00 | 0.96 | 1SG | 18 |
| ATOM | 18 | CB | GLU | 3 | −16.660 | −6.070 | 12.641 | 1.00 | 0.96 | 1SG | 19 |
| ATOM | 19 | CG | GLU | 3 | −15.544 | −6.526 | 13.580 | 1.00 | 0.96 | 1SG | 20 |
| ATOM | 20 | CD | GLU | 3 | −16.153 | −7.486 | 14.592 | 1.00 | 0.96 | 1SG | 21 |
| ATOM | 21 | OE1 | GLU | 3 | −16.963 | −7.024 | 15.440 | 1.00 | 0.96 | 1SG | 22 |
| ATOM | 22 | 0E2 | GLU | 3 | −15.817 | −8.698 | 14.526 | 1.00 | 0.96 | 1SG | 23 |
| ATOM | 23 | C | GLU | 3 | −17.019 | −3.825 | 13.565 | 1.00 | 0.96 | 1SG | 24 |
| ATOM | 24 | O | GLU | 3 | −16.811 | −3.047 | 12.636 | 1.00 | 0.96 | 1SG | 25 |
| ATOM | 25 | N | TYR | 4 | −16.692 | −3.531 | 14.837 | 1.00 | 0.95 | 1SG | 26 |
| ATOM | 26 | CA | TYR | 4 | −16.098 | −2.259 | 15.116 | 1.00 | 0.95 | 1SG | 27 |
| ATOM | 27 | CB | TYR | 4 | −15.798 | −2.021 | 16.604 | 1.00 | 0.95 | 1SG | 28 |
| ATOM | 28 | CG | TYR | 4 | −17.133 | −1.848 | 17.244 | 1.00 | 0.95 | 1SG | 29 |
| ATOM | 29 | CD1 | TYR | 4 | −17.758 | −0.622 | 17.220 | 1.00 | 0.95 | 1SG | 30 |
| ATOM | 30 | CD2 | TYR | 4 | −17.765 | −2.908 | 17.855 | 1.00 | 0.95 | 1SG | 31 |
| ATOM | 31 | CEl | TYR | 4 | −18.993 | −0.454 | 17.801 | 1.00 | 0.95 | 1SG | 32 |
| ATOM | 32 | CE2 | TYR | 4 | −19.000 | −2.745 | 18.438 | 1.00 | 0.95 | 1SG | 33 |
| ATOM | 33 | CZ | TYR | 4 | −19.615 | −1.517 | 18.412 | 1.00 | 0.95 | 1SG | 34 |
| ATOM | 34 | OH | TYR | 4 | −20.883 | −1.347 | 19.009 | 1.00 | 0.95 | 1SG | 35 |
| ATOM | 35 | C | TYR | 4 | −14.845 | −2.156 | 14.328 | 1.00 | 0.95 | 1SG | 36 |
| ATOM | 36 | O | TYR | 4 | −13.887 | −2.897 | 14.543 | 1.00 | 0.95 | 1SG | 37 |
| ATOM | 37 | N | SER | 5 | −14.838 | −1.211 | 13.373 | 1.00 | 0.66 | 1SG | 38 |
| ATOM | 38 | CA | SER | 5 | −13.708 | −1.076 | 12.513 | 1.00 | 0.66 | 1SG | 39 |
| ATOM | 39 | CB | SER | 5 | −14.097 | −1.057 | 11.033 | 1.00 | 0.66 | 1SG | 40 |
| ATOM | 40 | OG | SER | 5 | −14.626 | −2.320 | 10.658 | 1.00 | 0.66 | 1SG | 41 |
| ATOM | 41 | C | SER | 5 | −13.028 | 0.216 | 12.810 | 1.00 | 0.66 | 1SG | 42 |
| ATOM | 42 | O | SER | 5 | −13.667 | 1.260 | 12.937 | 1.00 | 0.66 | 1SG | 43 |
| ATOM | 43 | N | LEU | 6 | −11.690 | 0.158 | 12.936 | 1.00 | 0.52 | 1SG | 44 |
| ATOM | 44 | CA | LEU | 6 | −10.916 | 1.340 | 13.169 | 1.00 | 0.52 | 1SG | 45 |
| ATOM | 45 | CB | LEU | 6 | −9.827 | 1.172 | 14.248 | 1.00 | 0.52 | 1SG | 46 |
| ATOM | 46 | CG | LEU | 6 | −10.347 | 0.790 | 15.643 | 1.00 | 0.52 | 1SG | 47 |
| ATOM | 47 | CD2 | LEU | 6 | −11.485 | 1.718 | 16.096 | 1.00 | 0.52 | 1SG | 48 |
| ATOM | 48 | CD1 | LEU | 6 | −9.196 | 0.713 | 16.662 | 1.00 | 0.52 | 1SG | 49 |
| ATOM | 49 | C | LEU | 6 | −10.162 | 1.569 | 11.906 | 1.00 | 0.52 | 1SG | 50 |
| ATOM | 50 | O | LEU | 6 | −9.463 | 0.673 | 11.438 | 1.00 | 0.52 | 1SG | 51 |
| ATOM | 51 | N | SER | 7 | −10.284 | 2.768 | 11.308 | 1.00 | 0.52 | 1SG | 52 |
| ATOM | 52 | CA | SER | 7 | −9.549 | 3.039 | 10.106 | 1.00 | 0.52 | 1SG | 53 |
| ATOM | 53 | CB | SER | 7 | −10.274 | 3.973 | 9.120 | 1.00 | 0.52 | 1SG | 54 |
| ATOM | 54 | OG | SER | 7 | −11.470 | 3.371 | 8.653 | 1.00 | 0.52 | 1SG | 55 |
| ATOM | 55 | C | SER | 7 | −8.292 | 3.742 | 10.493 | 1.00 | 0.52 | 1SG | 56 |
| ATOM | 56 | O | SER | 7 | −8.233 | 4.441 | 11.505 | 1.00 | 0.52 | 1SG | 57 |
| ATOM | 57 | N | THR | 8 | −7.232 | 3.553 | 9.686 | 1.00 | 0.46 | 1SG | 58 |
| ATOM | 58 | CA | THR | 8 | −6.001 | 4.245 | 9.926 | 1.00 | 0.46 | 1SG | 59 |
| ATOM | 59 | CB | THR | 8 | −4.792 | 3.391 | 9.697 | 1.00 | 0.46 | 1SG | 60 |
| ATOM | 60 | OGI | THR | 8 | −4.813 | 2.272 | 10.570 | 1.00 | 0.46 | 1SG | 61 |
| ATOM | 61 | CG2 | THR | 8 | −3.522 | 4.231 | 9.927 | 1.00 | 0.46 | 1SG | 62 |
| ATOM | 62 | C | THR | 8 | −5.962 | 5.342 | 8.911 | 1.00 | 0.46 | 1SG | 63 |
| ATOM | 63 | O | THR | 8 | −6.141 | 5.093 | 7.719 | 1.00 | 0.46 | 1SG | 64 |
| ATOM | 64 | N | SER | 9 | 5.749 | 6.597 | 9.354 | 1.00 | 0.52 | 1SG | 65 |
| ATOM | 65 | CA | SER | 9 | −5.744 | 7.665 | 8.398 | 1.00 | 0.52 | 1SG | 66 |
| ATOM | 66 | CB | SER | 9 | −6.598 | 8.854 | 8.851 | 1.00 | 0.52 | 1SG | 67 |
| ATOM | 67 | OG | SER | 9 | −7.903 | 8.414 | 9.200 | 1.00 | 0.52 | 1SG | 68 |
| ATOM | 68 | C | SER | 9 | −4.342 | 8.172 | 8.247 | 1.00 | 0.52 | 1SG | 69 |
| ATOM | 69 | O | SER | 9 | −3.861 | 8.939 | 9.079 | 1.00 | 0.52 | 1SG | 70 |
| ATOM | 70 | N | PRO | 10 | −3.658 | 7.746 | 7.221 | 1.00 | 0.58 | 1SG | 71 |
| ATOM | 71 | CA | PRO | 10 | −2.321 | 8.260 | 7.044 | 1.00 | 0.58 | 1SG | 72 |
| ATOM | 72 | CD | PRO | 10 | −3.714 | 6.319 | 6.940 | 1.00 | 0.58 | 1SG | 73 |
| ATOM | 73 | CB | PRO | 10 | −1.523 | 7.160 | 6.354 | 1.00 | 0.58 | 1SG | 74 |
| ATOM | 74 | CG | PRO | 10 | −2.257 | 5.872 | 6.755 | 1.00 | 0.58 | 1SG | 75 |
| ATOM | 75 | C | PRO | 10 | −2.235 | 9.583 | 6.342 | 1.00 | 0.58 | 1SG | 76 |
| ATOM | 76 | O | PRO | 10 | −3.027 | 9.854 | 5.439 | 1.00 | 0.58 | 1SG | 77 |
| ATOM | 77 | N | PHE | 11 | −1.244 | 10.411 | 6.730 | 1.00 | 0.50 | 1SG | 78 |
| ATOM | 78 | CA | PHE | 11 | −1.029 | 11.683 | 6.104 | 1.00 | 0.50 | 1SG | 79 |
| ATOM | 79 | CB | PHE | 11 | −0.892 | 12.862 | 7.091 | 1.00 | 0.50 | 1SG | 80 |
| ATOM | 80 | CG | PHE | 11 | −2.248 | 13.249 | 7.575 | 1.00 | 0.50 | 1SG | 81 |
| ATOM | 81 | CD1 | PHE | 11 | −2.811 | 12.654 | 8.679 | 1.00 | 0.50 | 1SG | 82 |
| ATOM | 82 | CD2 | PHE | 11 | −2.958 | 14.218 | 6.907 | 1.00 | 0.50 | 1SG | 83 |
| ATOM | 83 | CE1 | PHE | 11 | −4.063 | 13.025 | 9.114 | 1.00 | 0.50 | 1SG | 84 |
| ATOM | 84 | CE2 | PHE | 11 | −4.208 | 14.598 | 7.335 | 1.00 | 0.50 | 1SG | 85 |
| ATOM | 85 | CZ | PHE | 11 | −4.763 | 13.995 | 8.439 | 1.00 | 0.50 | 1SG | 86 |
| ATOM | 86 | C | PHE | 11 | 0.254 | 11.590 | 5.336 | 1.00 | 0.50 | 1SG | 87 |
| ATOM | 87 | O | PHE | 11 | 1.244 | 11.047 | 5.828 | 1.00 | 0.50 | 1SG | 88 |
| ATOM | 88 | N | VAL | 12 | 0.251 | 12.108 | 4.089 | 1.00 | 0.74 | 1SG | 89 |
| ATOM | 89 | CA | VAL | 12 | 1.420 | 12.076 | 3.254 | 1.00 | 0.74 | 1SG | 90 |
| ATOM | 90 | CB | VAL | 12 | 1.293 | 11.170 | 2.076 | 1.00 | 0.74 | 1SG | 91 |
| ATOM | 91 | CG1 | VAL | 12 | 1.058 | 9.741 | 2.578 | 1.00 | 0.74 | 1SG | 92 |
| ATOM | 92 | CG2 | VAL | 12 | 0.177 | 11.711 | 1.184 | 1.00 | 0.74 | 1SG | 93 |
| ATOM | 93 | C | VAL | 12 | 1.620 | 13.447 | 2.689 | 1.00 | 0.74 | 1SG | 94 |
| ATOM | 94 | O | VAL | 12 | 0.708 | 14.274 | 2.712 | 1.00 | 0.74 | 1SG | 95 |
| ATOM | 95 | N | ARG | 13 | 2.827 | 13.710 | 2.140 | 1.00 | 1.09 | 1SG | 96 |
| ATOM | 96 | CA | ARG | 13 | 3.151 | 15.032 | 1.683 | 1.00 | 1.09 | 1SG | 97 |
| ATOM | 97 | CB | ARG | 13 | 3.906 | 15.805 | 2.793 | 1.00 | 1.09 | 1SG | 98 |
| ATOM | 98 | CG | ARG | 13 | 4.160 | 17.301 | 2.580 | 1.00 | 1.09 | 1SG | 99 |
| ATOM | 99 | CD | ARG | 13 | 2.886 | 18.134 | 2.530 | 1.00 | 1.09 | 1SG | 100 |
| ATOM | 100 | NE | ARG | 13 | 2.077 | 17.858 | 3.744 | 1.00 | 1.09 | 1SG | 101 |
| ATOM | 101 | CZ | ARG | 13 | 2.173 | 18.680 | 4.827 | 1.00 | 1.09 | 1SG | 102 |
| ATOM | 102 | NH1 | ARG | 13 | 2.964 | 19.794 | 4.798 | 1.00 | 1.09 | 1SG | 103 |
| ATOM | 103 | NH2 | ARG | 13 | 1.451 | 18.382 | 5.943 | 1.00 | 1.09 | 1SG | 104 |
| ATOM | 104 | C | ARG | 13 | 4.053 | 14.953 | 0.474 | 1.00 | 1.09 | 1SG | 105 |
| ATOM | 105 | O | ARG | 13 | 3.715 | 14.360 | −0.551 | 1.00 | 1.09 | 1SG | 106 |
| ATOM | 106 | N | ASN | 14 | 5.221 | 15.625 | 0.588 | 1.00 | 1.11 | 1SG | 107 |
| ATOM | 107 | CA | ASN | 14 | 6.254 | 15.806 | −0.398 | 1.00 | 1.11 | 1SG | 108 |
| ATOM | 108 | CB | ASN | 14 | 7.129 | 17.036 | −0.115 | 1.00 | 1.11 | 1SG | 109 |
| ATOM | 109 | CG | ASN | 14 | 6.273 | 18.277 | −0.307 | 1.00 | 1.11 | 1SG | 110 |
| ATOM | 110 | OD1 | ASN | 14 | 6.728 | 19.405 | −0.133 | 1.00 | 1.11 | 1SG | 111 |
| ATOM | 111 | ND2 | ASN | 14 | 4.981 | 18.060 | −0.661 | 1.00 | 1.11 | 1SG | 112 |
| ATOM | 112 | C | ASN | 14 | 7.227 | 14.678 | −0.628 | 1.00 | 1.11 | 1SG | 113 |
| ATOM | 113 | O | ASN | 14 | 7.532 | 14.398 | −1.785 | 1.00 | 1.11 | 1SG | 114 |
| ATOM | 114 | N | ARG | 15 | 7.785 | 13.989 | 0.398 | 1.00 | 0.97 | 1SG | 115 |
| ATOM | 115 | CA | ARG | 15 | 8.839 | 13.149 | −0.116 | 1.00 | 0.97 | 1SG | 116 |
| ATOM | 116 | CB | ARG | 15 | 10.232 | 13.793 | −0.248 | 1.00 | 0.97 | 1SG | 117 |
| ATOM | 117 | CG | ARG | 15 | 10.840 | 14.441 | 0.982 | 1.00 | 0.97 | 1SG | 118 |
| ATOM | 118 | CD | ARG | 15 | 12.021 | 15.339 | 0.603 | 1.00 | 0.97 | 1SG | 119 |
| ATOM | 119 | NE | ARG | 15 | 11.463 | 16.533 | −0.098 | 1.00 | 0.97 | 1SG | 120 |
| ATOM | 120 | CZ | ARG | 15 | 12.232 | 17.238 | −0.981 | 1.00 | 0.97 | 1SG | 121 |
| ATOM | 121 | NH1 | ARG | 15 | 13.522 | 16.854 | −1.199 | 1.00 | 0.97 | 1SG | 122 |
| ATOM | 122 | NH2 | ARG | 15 | 11.709 | 18.306 | −1.655 | 1.00 | 0.97 | 1SG | 123 |
| ATOM | 123 | C | ARG | 15 | 8.912 | 11.713 | 0.308 | 1.00 | 0.97 | 1SG | 124 |
| ATOM | 124 | O | ARG | 15 | 8.521 | 11.289 | 1.394 | 1.00 | 0.97 | 1SG | 125 |
| ATOM | 125 | N | TYR | 16 | 9.551 | 10.990 | −0.636 | 1.00 | 0.67 | 1SG | 126 |
| ATOM | 126 | CA | TYR | 16 | 9.641 | 9.600 | −0.990 | 1.00 | 0.67 | 1SG | 127 |
| ATOM | 127 | CB | TYR | 16 | 10.723 | 9.386 | −2.072 | 1.00 | 0.67 | 1SG | 128 |
| ATOM | 128 | CG | TYR | 16 | 12.059 | 9.854 | −1.607 | 1.00 | 0.67 | 1SG | 129 |
| ATOM | 129 | CD1 | TYR | 16 | 12.919 | 9.016 | −0.933 | 1.00 | 0.67 | 1SG | 130 |
| ATOM | 130 | CD2 | TYR | 16 | 12.460 | 11.142 | −1.865 | 1.00 | 0.67 | 1SG | 131 |
| ATOM | 131 | CE1 | TYR | 16 | 14.155 | 9.454 | −0.515 | 1.00 | 0.67 | 1SG | 132 |
| ATOM | 132 | CE2 | TYR | 16 | 13.692 | 11.590 | −1.449 | 1.00 | 0.67 | 1SG | 133 |
| ATOM | 133 | CZ | TYR | 16 | 14.541 | 10.747 | −0.774 | 1.00 | 0.67 | 1SG | 134 |
| ATOM | 134 | OH | TYR | 16 | 15.805 | 11.212 | −0.351 | 1.00 | 0.67 | 1SG | 135 |
| ATOM | 135 | C | TYR | 16 | 9.943 | 8.608 | 0.078 | 1.00 | 0.67 | 1SG | 136 |
| ATOM | 136 | O | TYR | 16 | 9.313 | 7.551 | 0.081 | 1.00 | 0.67 | 1SG | 137 |
| ATOM | 137 | N | THR | 17 | 10.875 | 8.889 | 0.999 | 1.00 | 0.41 | 1SG | 138 |
| ATOM | 138 | CA | THR | 17 | 11.330 | 7.852 | 1.882 | 1.00 | 0.41 | 1SG | 139 |
| ATOM | 139 | CB | THR | 17 | 12.262 | 8.348 | 2.956 | 1.00 | 0.41 | 1SG | 140 |
| ATOM | 140 | OG1 | THR | 17 | 11.598 | 9.304 | 3.775 | 1.00 | 0.41 | 1SG | 141 |
| ATOM | 141 | CG2 | THR | 17 | 13.528 | 8.949 | 2.322 | 1.00 | 0.41 | 1SG | 142 |
| ATOM | 142 | C | THR | 17 | 10.204 | 7.230 | 2.626 | 1.00 | 0.41 | 1SG | 143 |
| ATOM | 143 | O | THR | 17 | 10.071 | 6.008 | 2.672 | 1.00 | 0.41 | 1SG | 144 |
| ATOM | 144 | N | HIS | 18 | 9.322 | 8.053 | 3.192 | 1.00 | 0.48 | 1SG | 145 |
| ATOM | 145 | CA | HIS | 18 | 8.418 | 7.457 | 4.108 | 1.00 | 0.48 | 1SG | 146 |
| ATOM | 146 | ND1 | HIS | 18 | 5.573 | 7.412 | 5.851 | 1.00 | 0.48 | 1SG | 147 |
| ATOM | 147 | CG | HIS | 18 | 6.931 | 7.588 | 5.994 | 1.00 | 0.48 | 1SG | 148 |
| ATOM | 148 | CB | HIS | 18 | 7.778 | 8.418 | 5.095 | 1.00 | 0.48 | 1SG | 149 |
| ATOM | 149 | NE2 | HIS | 18 | 6.187 | 6.188 | 7.592 | 1.00 | 0.48 | 1SG | 150 |
| ATOM | 150 | CD2 | HIS | 18 | 7.291 | 6.839 | 7.073 | 1.00 | 0.48 | 1SG | 151 |
| ATOM | 151 | CE1 | HIS | 18 | 5.179 | 6.568 | 6.830 | 1.00 | 0.48 | 1SG | 152 |
| ATOM | 152 | C | HIS | 18 | 7.286 | 6.676 | 3.531 | 1.00 | 0.48 | 1SG | 153 |
| ATOM | 153 | O | HIS | 18 | 7.020 | 5.592 | 4.030 | 1.00 | 0.48 | 1SG | 154 |
| ATOM | 154 | N | VAL | 19 | 6.605 | 7.133 | 2.464 | 1.00 | 0.61 | 1SG | 155 |
| ATOM | 155 | CA | VAL | 19 | 5.329 | 6.518 | 2.169 | 1.00 | 0.61 | 1SG | 156 |
| ATOM | 156 | CB | VAL | 19 | 4.543 | 7.113 | 1.042 | 1.00 | 0.61 | 1SG | 157 |
| ATOM | 157 | CG1 | VAL | 19 | 4.360 | 8.613 | 1.298 | 1.00 | 0.61 | 1SG | 158 |
| ATOM | 158 | CG2 | VAL | 19 | 5.175 | 6.704 | −0.295 | 1.00 | 0.61 | 1SG | 159 |
| ATOM | 159 | C | VAL | 19 | 5.425 | 5.063 | 1.844 | 1.00 | 0.61 | 1SG | 160 |
| ATOM | 160 | O | VAL | 19 | 4.441 | 4.340 | 1.985 | 1.00 | 0.61 | 1SG | 161 |
| ATOM | 161 | N | LYS | 20 | 6.559 | 4.602 | 1.300 | 1.00 | 0.71 | 1SG | 162 |
| ATOM | 162 | CA | LYS | 20 | 6.676 | 3.204 | 0.987 | 1.00 | 0.71 | 1SG | 163 |
| ATOM | 163 | CB | LYS | 20 | 7.954 | 2.897 | 0.193 | 1.00 | 0.71 | 1SG | 164 |
| ATOM | 164 | CG | LYS | 20 | 9.237 | 3.328 | 0.905 | 1.00 | 0.71 | 1SG | 165 |
| ATOM | 165 | CD | LYS | 20 | 10.502 | 2.855 | 0.188 | 1.00 | 0.71 | 1SG | 166 |
| ATOM | 166 | CE | LYS | 20 | 11.801 | 3.392 | 0.791 | 1.00 | 0.71 | 1SG | 167 |
| ATOM | 167 | NZ | LYS | 20 | 12.958 | 2.855 | 0.040 | 1.00 | 0.71 | 1SG | 168 |
| ATOM | 168 | C | LYS | 20 | 6.715 | 2.407 | 2.256 | 1.00 | 0.71 | 1SG | 169 |
| ATOM | 169 | O | LYS | 20 | 6.140 | 1.323 | 2.356 | 1.00 | 0.71 | 1SG | 170 |
| ATOM | 170 | N | ASP | 21 | 7.378 | 2.981 | 3.269 | 1.00 | 1.15 | 1SG | 171 |
| ATOM | 171 | CA | ASP | 21 | 7.688 | 2.425 | 4.555 | 1.00 | 1.15 | 1SG | 172 |
| ATOM | 172 | CB | ASP | 21 | 7.797 | 3.512 | 5.625 | 1.00 | 1.15 | 1SG | 173 |
| ATOM | 173 | CG | ASP | 21 | 9.091 | 4.275 | 5.366 | 1.00 | 1.15 | 1SG | 174 |
| ATOM | 174 | OD1 | ASP | 21 | 9.857 | 3.841 | 4.467 | 1.00 | 1.15 | 1SG | 175 |
| ATOM | 175 | OD2 | ASP | 21 | 9.349 | 5.288 | 6.065 | 1.00 | 1.15 | 1SG | 176 |
| ATOM | 176 | C | ASP | 21 | 6.854 | 1.239 | 5.021 | 1.00 | 1.15 | 1SG | 177 |
| ATOM | 177 | O | ASP | 21 | 7.272 | 0.119 | 4.745 | 1.00 | 1.15 | 1SG | 178 |
| ATOM | 178 | N | GLU | 22 | 5.679 | 1.320 | 5.712 | 1.00 | 1.55 | 1SG | 179 |
| ATOM | 179 | CA | GLU | 22 | 4.770 | 2.373 | 6.061 | 1.00 | 1.55 | 1SG | 180 |
| ATOM | 180 | CB | GLU | 22 | 5.287 | 3.385 | 7.100 | 1.00 | 1.55 | 1SG | 181 |
| ATOM | 181 | CG | GLU | 22 | 5.940 | 2.760 | 8.333 | 1.00 | 1.55 | 1SG | 182 |
| ATOM | 182 | CD | GLU | 22 | 4.882 | 2.178 | 9.250 | 1.00 | 1.55 | 1SG | 183 |
| ATOM | 183 | OE1 | GLU | 22 | 3.671 | 2.269 | 8.915 | 1.00 | 1.55 | 1SG | 184 |
| ATOM | 184 | OE2 | GLU | 22 | 5.286 | 1.632 | 10.310 | 1.00 | 1.55 | 1SG | 185 |
| ATOM | 185 | C | GLU | 22 | 4.416 | 3.124 | 4.848 | 1.00 | 1.55 | 1SG | 186 |
| ATOM | 186 | O | GLU | 22 | 5.198 | 3.900 | 4.331 | 1.00 | 1.55 | 1SG | 187 |
| ATOM | 187 | N | VAL | 23 | 3.192 | 2.950 | 4.347 | 1.00 | 1.21 | 1SG | 188 |
| ATOM | 188 | CA | VAL | 23 | 2.130 | 2.211 | 4.953 | 1.00 | 1.21 | 1SG | 189 |
| ATOM | 189 | CB | VAL | 23 | 0.853 | 2.303 | 4.175 | 1.00 | 1.21 | 1SG | 190 |
| ATOM | 190 | CG1 | VAL | 23 | 0.388 | 3.771 | 4.172 | 1.00 | 1.21 | 1SG | 191 |
| ATOM | 191 | CG2 | VAL | 23 | 1.098 | 1.724 | 2.772 | 1.00 | 1.21 | 1SG | 192 |
| ATOM | 192 | C | VAL | 23 | 2.483 | 0.774 | 5.084 | 1.00 | 1.21 | 1SG | 193 |
| ATOM | 193 | O | VAL | 23 | 1.828 | 0.041 | 5.822 | 1.00 | 1.21 | 1SG | 194 |
| ATOM | 194 | N | ARG | 24 | 3.523 | 0.327 | 4.367 | 1.00 | 0.64 | 1SG | 195 |
| ATOM | 195 | CA | ARG | 24 | 3.833 | −1.070 | 4.326 | 1.00 | 0.64 | 1SG | 196 |
| ATOM | 196 | CB | ARG | 24 | 5.120 | −1.356 | 3.542 | 1.00 | 0.64 | 1SG | 197 |
| ATOM | 197 | CG | ARG | 24 | 5.428 | −2.847 | 3.412 | 1.00 | 0.64 | 1SG | 198 |
| ATOM | 198 | CD | ARG | 24 | 6.563 | −3.150 | 2.434 | 1.00 | 0.64 | 1SG | 199 |
| ATOM | 199 | NE | ARG | 24 | 6.863 | −4.605 | 2.533 | 1.00 | 0.64 | 1SG | 200 |
| ATOM | 200 | CZ | ARG | 24 | 7.794 | −5.030 | 3.436 | 1.00 | 0.64 | 1SG | 201 |
| ATOM | 201 | NH1 | ARG | 24 | 8.432 | −4.123 | 4.232 | 1.00 | 0.64 | 1SG | 202 |
| ATOM | 202 | NH2 | ARG | 24 | 8.084 | −6.359 | 3.538 | 1.00 | 0.64 | 1SG | 203 |
| ATOM | 203 | C | ARG | 24 | 3.972 | −1.658 | 5.705 | 1.00 | 0.64 | 1SG | 204 |
| ATOM | 204 | O | ARG | 24 | 3.476 | −2.755 | 5.950 | 1.00 | 0.64 | 1SG | 205 |
| ATOM | 205 | N | TYR | 25 | 4.629 | −0.965 | 6.653 | 1.00 | 0.45 | 1SG | 206 |
| ATOM | 206 | CA | TYR | 25 | 4.824 | −1.546 | 7.949 | 1.00 | 0.45 | 1SG | 207 |
| ATOM | 207 | CB | TYR | 25 | 5.685 | −0.675 | 8.869 | 1.00 | 0.45 | 1SG | 208 |
| ATOM | 208 | CG | TYR | 25 | 6.187 | −1.619 | 9.897 | 1.00 | 0.45 | 1SG | 209 |
| ATOM | 209 | CD1 | TYR | 25 | 5.378 | −2.053 | 10.916 | 1.00 | 0.45 | 1SG | 210 |
| ATOM | 210 | CD2 | TYR | 25 | 7.482 | −2.076 | 9.826 | 1.00 | 0.45 | 1SG | 211 |
| ATOM | 211 | CE1 | TYR | 25 | 5.868 | −2.931 | 11.850 | 1.00 | 0.45 | 1SG | 212 |
| ATOM | 212 | CE2 | TYR | 25 | 7.977 | −2.954 | 10.759 | 1.00 | 0.45 | 1SG | 213 |
| ATOM | 213 | CZ | TYR | 25 | 7.162 | −3.382 | 11.775 | 1.00 | 0.45 | 1SG | 214 |
| ATOM | 214 | OH | TYR | 25 | 7.650 | −4.284 | 12.742 | 1.00 | 0.45 | 1SG | 215 |
| ATOM | 215 | C | TYR | 25 | 3.485 | −1.721 | 8.596 | 1.00 | 0.45 | 1SG | 216 |
| ATOM | 216 | O | TYR | 25 | 3.226 | −2.742 | 9.228 | 1.00 | 0.45 | 1SG | 217 |
| ATOM | 217 | N | GLU | 26 | 2.592 | −0.722 | 8.450 | 1.00 | 0.36 | 1SG | 218 |
| ATOM | 218 | CA | GLU | 26 | 1.279 | −0.795 | 9.031 | 1.00 | 0.36 | 1SG | 219 |
| ATOM | 219 | CB | GLU | 26 | 0.412 | 0.439 | 8.732 | 1.00 | 0.36 | 1SG | 220 |
| ATOM | 220 | CG | GLU | 26 | −0.989 | 0.350 | 9.345 | 1.00 | 0.36 | 1SG | 221 |
| ATOM | 221 | CD | GLU | 26 | −1.830 | 1.468 | 8.747 | 1.00 | 0.36 | 1SG | 222 |
| ATOM | 222 | OE1 | GLU | 26 | −1.226 | 2.445 | 8.228 | 1.00 | 0.36 | 1SG | 223 |
| ATOM | 223 | OE2 | GLU | 26 | −3.085 | 1.359 | 8.794 | 1.00 | 0.36 | 1SG | 224 |
| ATOM | 224 | C | GLU | 26 | 0.561 | −1.955 | 8.424 | 1.00 | 0.36 | 1SG | 225 |
| ATOM | 225 | O | GLU | 26 | −0.114 | −2.709 | 9.122 | 1.00 | 0.36 | 1SG | 226 |
| ATOM | 226 | N | VAL | 27 | 0.693 | −2.127 | 7.097 | 1.00 | 0.19 | 1SG | 227 |
| ATOM | 227 | CA | VAL | 27 | 0.012 | −3.188 | 6.416 | 1.00 | 0.19 | 1SG | 228 |
| ATOM | 228 | CB | VAL | 27 | 0.239 | −3.179 | 4.932 | 1.00 | 0.19 | 1SG | 229 |
| ATOM | 229 | CG1 | VAL | 27 | −0.425 | −4.431 | 4.333 | 1.00 | 0.19 | 1SG | 230 |
| ATOM | 230 | CG2 | VAL | 27 | −0.302 | −1.858 | 4.359 | 1.00 | 0.19 | 1SG | 231 |
| ATOM | 231 | C | VAL | 27 | 0.524 | −4.489 | 6.937 | 1.00 | 0.19 | 1SG | 232 |
| ATOM | 232 | O | VAL | 27 | −0.233 | −5.446 | 7.083 | 1.00 | 0.19 | 1SG | 233 |
| ATOM | 233 | N | ASN | 28 | 1.834 | −4.564 | 7.224 | 1.00 | 0.16 | 1SG | 234 |
| ATOM | 234 | CA | ASN | 28 | 2.408 | −5.791 | 7.694 | 1.00 | 0.16 | 1SG | 235 |
| ATOM | 235 | CB | ASN | 28 | 3.930 | −5.684 | 7.894 | 1.00 | 0.16 | 1SG | 236 |
| ATOM | 236 | CG | ASN | 28 | 4.473 | −7.035 | 8.344 | 1.00 | 0.16 | 1SG | 237 |
| ATOM | 237 | OD1 | ASN | 28 | 4.125 | −7.543 | 9.409 | 1.00 | 0.16 | 1SG | 238 |
| ATOM | 238 | ND2 | ASN | 28 | 5.359 | −7.635 | 7.507 | 1.00 | 0.16 | 1SG | 239 |
| ATOM | 239 | C | ASN | 28 | 1.792 | −6.152 | 9.013 | 1.00 | 0.16 | 1SG | 240 |
| ATOM | 240 | O | ASN | 28 | 1.399 | −7.297 | 9.226 | 1.00 | 0.16 | 1SG | 241 |
| ATOM | 241 | N | CYS | 29 | 1.692 | −5.172 | 9.931 | 1.00 | 0.40 | 1SG | 242 |
| ATOM | 242 | CA | CYS | 29 | 1.183 | −5.376 | 11.260 | 1.00 | 0.40 | 1SG | 243 |
| ATOM | 243 | CB | CYS | 29 | 1.408 | −4.172 | 12.193 | 1.00 | 0.40 | 1SG | 244 |
| ATOM | 244 | SG | CYS | 29 | 3.135 | −4.012 | 12.721 | 1.00 | 0.40 | 1SG | 245 |
| ATOM | 245 | C | CYS | 29 | −0.290 | −5.643 | 11.280 | 1.00 | 0.40 | 1SG | 246 |
| ATOM | 246 | O | CYS | 29 | −0.744 | −6.395 | 12.141 | 1.00 | 0.40 | 1SG | 247 |
| ATOM | 247 | N | SER | 30 | −1.041 | −5.042 | 10.329 | 1.00 | 0.66 | 1SG | 248 |
| ATOM | 248 | CA | SER | 30 | −2.483 | −4.988 | 10.223 | 1.00 | 0.66 | 1SG | 249 |
| ATOM | 249 | CB | SER | 30 | −2.975 | −5.079 | 8.764 | 1.00 | 0.66 | 1SG | 250 |
| ATOM | 250 | OG | SER | 30 | −4.394 | −5.020 | 8.728 | 1.00 | 0.66 | 1SG | 251 |
| ATOM | 251 | C | SER | 30 | −3.179 | −6.057 | 11.007 | 1.00 | 0.66 | 1SG | 252 |
| ATOM | 252 | O | SER | 30 | −3.699 | −5.786 | 12.086 | 1.00 | 0.66 | 1SG | 253 |
| ATOM | 253 | N | GLY | 31 | −3.215 | −7.302 | 10.504 | 1.00 | 0.81 | 1SG | 254 |
| ATOM | 254 | CA | GLY | 31 | −3.877 | −8.325 | 11.262 | 1.00 | 0.81 | 1SG | 255 |
| ATOM | 255 | C | GLY | 31 | −5.074 | −8.737 | 10.474 | 1.00 | 0.81 | 1SG | 256 |
| ATOM | 256 | O | GLY | 31 | −5.171 | −9.876 | 10.021 | 1.00 | 0.81 | 1SG | 257 |
| ATOM | 257 | N | ILE | 32 | −6.039 | −7.813 | 10.301 | 1.00 | 1.05 | 1SG | 258 |
| ATOM | 258 | CA | ILE | 32 | −7.181 | −8.163 | 9.515 | 1.00 | 1.05 | 1SG | 259 |
| ATOM | 259 | CB | ILE | 32 | −8.468 | −8.129 | 10.307 | 1.00 | 1.05 | 1SG | 260 |
| ATOM | 260 | CG2 | ILE | 32 | −8.793 | −6.681 | 10.702 | 1.00 | 1.05 | 1SG | 261 |
| ATOM | 261 | CG1 | ILE | 32 | −9.589 | −8.892 | 9.582 | 1.00 | 1.05 | 1SG | 262 |
| ATOM | 262 | CD1 | ILE | 32 | −9.914 | −8.363 | 8.193 | 1.00 | 1.05 | 1SG | 263 |
| ATOM | 263 | C | ILE | 32 | −7.196 | −7.223 | 8.342 | 1.00 | 1.05 | 1SG | 264 |
| ATOM | 264 | O | ILE | 32 | −7.355 | −6.009 | 8.477 | 1.00 | 1.05 | 1SG | 265 |
| ATOM | 265 | N | TYR | 33 | −6.989 | −7.797 | 7.142 | 1.00 | 1.19 | 1SG | 266 |
| ATOM | 266 | CA | TYR | 33 | −6.859 | −7.066 | 5.917 | 1.00 | 1.19 | 1SG | 267 |
| ATOM | 267 | CB | TYR | 33 | −6.388 | −7.955 | 4.750 | 1.00 | 1.19 | 1SG | 268 |
| ATOM | 268 | CG | TYR | 33 | −7.323 | −9.111 | 4.623 | 1.00 | 1.19 | 1SG | 269 |
| ATOM | 269 | CD1 | TYR | 33 | −7.206 | −10.196 | 5.462 | 1.00 | 1.19 | 1SG | 270 |
| ATOM | 270 | CD2 | TYR | 33 | −8.305 | −9.125 | 3.660 | 1.00 | 1.19 | 1SG | 271 |
| ATOM | 271 | CE1 | TYR | 33 | −8.059 | −11.268 | 5.351 | 1.00 | 1.19 | 1SG | 272 |
| ATOM | 272 | CE2 | TYR | 33 | −9.162 | −10.195 | 3.543 | 1.00 | 1.19 | 1SG | 273 |
| ATOM | 273 | CZ | TYR | 33 | −9.040 | −11.269 | 4.390 | 1.00 | 1.19 | 1SG | 274 |
| ATOM | 274 | OH | TYR | 33 | −9.915 | −12.370 | 4.276 | 1.00 | 1.19 | 1SG | 275 |
| ATOM | 275 | C | TYR | 33 | −8.138 | −6.412 | 5.527 | 1.00 | 1.19 | 1SG | 276 |
| ATOM | 276 | O | TYR | 33 | −8.107 | −5.279 | 5.048 | 1.00 | 1.19 | 1SG | 277 |
| ATOM | 277 | N | GLU | 34 | −9.274 | −7.114 | 5.747 | 1.00 | 0.97 | 1SG | 278 |
| ATOM | 278 | CA | GLU | 34 | −10.595 | −6.712 | 5.341 | 1.00 | 0.97 | 1SG | 279 |
| ATOM | 279 | CB | GLU | 34 | −11.742 | −7.422 | 6.081 | 1.00 | 0.97 | 1SG | 280 |
| ATOM | 280 | CG | GLU | 34 | −11.872 | −8.895 | 5.686 | 1.00 | 0.97 | 1SG | 281 |
| ATOM | 281 | CD | GLU | 34 | −12.191 | −8.946 | 4.197 | 1.00 | 0.97 | 1SG | 282 |
| ATOM | 282 | OE1 | GLU | 34 | −11.323 | −8.508 | 3.396 | 1.00 | 0.97 | 1SG | 283 |
| ATOM | 283 | OE2 | GLU | 34 | −13.304 | −9.418 | 3.840 | 1.00 | 0.97 | 1SG | 284 |
| ATOM | 284 | C | GLU | 34 | −10.785 | −5.241 | 5.449 | 1.00 | 0.97 | 1SG | 285 |
| ATOM | 285 | O | GLU | 34 | −10.324 | −4.591 | 6.387 | 1.00 | 0.97 | 1SG | 286 |
| ATOM | 286 | N | GLN | 35 | −11.478 | −4.695 | 4.437 | 1.00 | 0.56 | 1SG | 287 |
| ATOM | 287 | CA | GLN | 35 | −11.681 | −3.291 | 4.291 | 1.00 | 0.56 | 1SG | 288 |
| ATOM | 288 | CB | GLN | 35 | −12.600 | −2.973 | 3.099 | 1.00 | 0.56 | 1SG | 289 |
| ATOM | 289 | CG | GLN | 35 | −12.032 | −3.433 | 1.753 | 1.00 | 0.56 | 1SG | 290 |
| ATOM | 290 | CD | GLN | 35 | −13.100 | −3.218 | 0.689 | 1.00 | 0.56 | 1SG | 291 |
| ATOM | 291 | OE1 | GLN | 35 | −13.855 | −2.248 | 0.733 | 1.00 | 0.56 | 1SG | 292 |
| ATOM | 292 | NE2 | GLN | 35 | −13.176 | −4.158 | −0.292 | 1.00 | 0.56 | 1SG | 293 |
| ATOM | 293 | C | GLN | 35 | −12.350 | −2.781 | 5.519 | 1.00 | 0.56 | 1SG | 294 |
| ATOM | 294 | O | GLN | 35 | −11.929 | −1.776 | 6.090 | 1.00 | 0.56 | 1SG | 295 |
| ATOM | 295 | N | GLU | 36 | −13.408 | −3.471 | 5.978 | 1.00 | 0.31 | 1SG | 296 |
| ATOM | 296 | CA | GLU | 36 | −14.111 | −2.944 | 7.107 | 1.00 | 0.31 | 1SG | 297 |
| ATOM | 297 | CB | GLU | 36 | −15.324 | −3.794 | 7.534 | 1.00 | 0.31 | 1SG | 298 |
| ATOM | 298 | CG | GLU | 36 | −16.463 | −3.777 | 6.509 | 1.00 | 0.31 | 1SG | 299 |
| ATOM | 299 | CD | GLU | 36 | −17.687 | −4.440 | 7.131 | 1.00 | 0.31 | 1SG | 300 |
| ATOM | 300 | OE1 | GLU | 36 | −18.192 | −3.906 | 8.154 | 1.00 | 0.31 | 1SG | 301 |
| ATOM | 301 | OE2 | GLU | 36 | −18.136 | −5.486 | 6.590 | 1.00 | 0.31 | 1SG | 302 |
| ATOM | 302 | C | GLU | 36 | −13.206 | −2.799 | 8.296 | 1.00 | 0.31 | 1SG | 303 |
| ATOM | 303 | O | GLU | 36 | −12.895 | −1.665 | 8.654 | 1.00 | 0.31 | 1SG | 304 |
| ATOM | 304 | N | PRO | 37 | −12.728 | −3.874 | 8.891 | 1.00 | 0.42 | 1SG | 305 |
| ATOM | 305 | CA | PRO | 37 | −11.976 | −3.806 | 10.123 | 1.00 | 0.42 | 1SG | 306 |
| ATOM | 306 | CD | PRO | 37 | −12.457 | −5.107 | 8.170 | 1.00 | 0.42 | 1SG | 307 |
| ATOM | 307 | CB | PRO | 37 | −11.414 | −5.204 | 10.351 | 1.00 | 0.42 | 1SG | 308 |
| ATOM | 308 | CG | PRO | 37 | −11.298 | −5.772 | 8.928 | 1.00 | 0.42 | 1SG | 309 |
| ATOM | 309 | C | PRO | 37 | −10.910 | −2.760 | 10.175 | 1.00 | 0.42 | 1SG | 310 |
| ATOM | 310 | O | PRO | 37 | −11.012 | −1.867 | 11.014 | 1.00 | 0.42 | 1SG | 311 |
| ATOM | 311 | N | LEU | 38 | −9.881 | −2.831 | 9.313 | 1.00 | 0.70 | 1SG | 312 |
| ATOM | 312 | CA | LEU | 38 | −8.905 | −1.786 | 9.358 | 1.00 | 0.70 | 1SG | 313 |
| ATOM | 313 | CB | LEU | 38 | −7.555 | −2.200 | 9.957 | 1.00 | 0.70 | 1SG | 314 |
| ATOM | 314 | CG | LEU | 38 | −6.499 | −1.083 | 9.824 | 1.00 | 0.70 | 1SG | 315 |
| ATOM | 315 | CD2 | LEU | 38 | −5.091 | −1.595 | 10.162 | 1.00 | 0.70 | 1SG | 316 |
| ATOM | 316 | CD1 | LEU | 38 | −6.895 | 0.170 | 10.623 | 1.00 | 0.70 | 1SG | 317 |
| ATOM | 317 | C | LEU | 38 | −8.613 | −1.380 | 7.961 | 1.00 | 0.70 | 1SG | 318 |
| ATOM | 318 | O | LEU | 38 | −7.712 | −1.926 | 7.328 | 1.00 | 0.70 | 1SG | 319 |
| ATOM | 319 | N | GLU | 39 | −9.353 | −0.395 | 7.430 | 1.00 | 0.71 | 1SG | 320 |
| ATOM | 320 | CA | GLU | 39 | −8.987 | 0.004 | 6.113 | 1.00 | 0.71 | 1SG | 321 |
| ATOM | 321 | CB | GLU | 39 | −10.147 | 0.460 | 5.212 | 1.00 | 0.71 | 1SG | 322 |
| ATOM | 322 | CG | GLU | 39 | −9.667 | 0.862 | 3.813 | 1.00 | 0.71 | 1SG | 323 |
| ATOM | 323 | CD | GLU | 39 | −9.207 | −0.399 | 3.087 | 1.00 | 0.71 | 1SG | 324 |
| ATOM | 324 | OE1 | GLU | 39 | −9.642 | −1.509 | 3.493 | 1.00 | 0.71 | 1SG | 325 |
| ATOM | 325 | OE2 | GLU | 39 | −8.414 | −0.268 | 2.117 | 1.00 | 0.71 | 1SG | 326 |
| ATOM | 326 | C | GLU | 39 | −8.058 | 1.151 | 6.276 | 1.00 | 0.71 | 1SG | 327 |
| ATOM | 327 | O | GLU | 39 | −8.122 | 1.877 | 7.267 | 1.00 | 0.71 | 1SG | 328 |
| ATOM | 328 | N | ILE | 40 | −7.144 | 1.332 | 5.310 | 1.00 | 0.43 | 1SG | 329 |
| ATOM | 329 | CA | ILE | 40 | −6.229 | 2.422 | 5.423 | 1.00 | 0.43 | 1SG | 330 |
| ATOM | 330 | CB | ILE | 40 | −4.801 | 2.041 | 5.144 | 1.00 | 0.43 | 1SG | 331 |
| ATOM | 331 | CG2 | ILE | 40 | −3.975 | 3.334 | 5.060 | 1.00 | 0.43 | 1SG | 332 |
| ATOM | 332 | CG1 | ILE | 40 | −4.281 | 1.052 | 6.202 | 1.00 | 0.43 | 1SG | 333 |
| ATOM | 333 | CD1 | ILE | 40 | −4.963 | −0.315 | 6.165 | 1.00 | 0.43 | 1SG | 334 |
| ATOM | 334 | C | ILE | 40 | −6.621 | 3.447 | 4.412 | 1.00 | 0.43 | 1SG | 335 |
| ATOM | 335 | O | ILE | 40 | −6.815 | 3.137 | 3.237 | 1.00 | 0.43 | 1SG | 336 |
| ATOM | 336 | N | GLY | 41 | −6.769 | 4.706 | 4.864 | 1.00 | 0.27 | 1SG | 337 |
| ATOM | 337 | CA | GLY | 41 | −7.086 | 5.777 | 3.965 | 1.00 | 0.27 | 1SG | 338 |
| ATOM | 338 | C | GLY | 41 | −5.914 | 6.702 | 4.002 | 1.00 | 0.27 | 1SG | 339 |
| ATOM | 339 | O | GLY | 41 | −5.584 | 7.255 | 5.049 | 1.00 | 0.27 | 1SG | 340 |
| ATOM | 340 | N | LYS | 42 | −5.259 | 6.915 | 2.844 | 1.00 | 0.27 | 1SG | 341 |
| ATOM | 341 | CA | LYS | 42 | −4.072 | 7.721 | 2.848 | 1.00 | 0.27 | 1SG | 342 |
| ATOM | 342 | CB | LYS | 42 | −2.939 | 7.035 | 2.065 | 1.00 | 0.27 | 1SG | 343 |
| ATOM | 343 | CG | LYS | 42 | −1.531 | 7.523 | 2.396 | 1.00 | 0.27 | 1SG | 344 |
| ATOM | 344 | CD | LYS | 42 | −0.457 | 6.581 | 1.845 | 1.00 | 0.27 | 1SG | 345 |
| ATOM | 345 | CE | LYS | 42 | 0.932 | 6.806 | 2.443 | 1.00 | 0.27 | 1SG | 346 |
| ATOM | 346 | NZ | LYS | 42 | 1.895 | 5.834 | 1.880 | 1.00 | 0.27 | 1SG | 347 |
| ATOM | 347 | C | LYS | 42 | −4.380 | 9.030 | 2.187 | 1.00 | 0.27 | 1SG | 348 |
| ATOM | 348 | O | LYS | 42 | −4.669 | 9.070 | 0.992 | 1.00 | 0.27 | 1SG | 349 |
| ATOM | 349 | N | SER | 43 | −4.323 | 10.142 | 2.957 | 1.00 | 0.43 | 1SG | 350 |
| ATOM | 350 | CA | SER | 43 | −4.580 | 11.433 | 2.381 | 1.00 | 0.43 | 1SG | 351 |
| ATOM | 351 | CB | SER | 43 | −5.064 | 12.529 | 3.352 | 1.00 | 0.43 | 1SG | 352 |
| ATOM | 352 | OG | SER | 43 | −5.270 | 13.760 | 2.668 | 1.00 | 0.43 | 1SG | 353 |
| ATOM | 353 | C | SER | 43 | −3.299 | 11.935 | 1.836 | 1.00 | 0.43 | 1SG | 354 |
| ATOM | 354 | O | SER | 43 | −2.284 | 11.970 | 2.529 | 1.00 | 0.43 | 1SG | 355 |
| ATOM | 355 | N | LEU | 44 | −3.344 | 12.351 | 0.562 | 1.00 | 0.66 | 1SG | 356 |
| ATOM | 356 | CA | LEU | 44 | −2.195 | 12.852 | −0.118 | 1.00 | 0.66 | 1SG | 357 |
| ATOM | 357 | CB | LEU | 44 | −2.150 | 12.421 | −1.595 | 1.00 | 0.66 | 1SG | 358 |
| ATOM | 358 | CG | LEU | 44 | −2.111 | 10.903 | −1.847 | 1.00 | 0.66 | 1SG | 359 |
| ATOM | 359 | CD2 | LEU | 44 | −0.956 | 10.227 | −1.092 | 1.00 | 0.66 | 1SG | 360 |
| ATOM | 360 | CD1 | LEU | 44 | −2.079 | 10.600 | −3.355 | 1.00 | 0.66 | 1SG | 361 |
| ATOM | 361 | C | LEU | 44 | −2.354 | 14.330 | −0.166 | 1.00 | 0.66 | 1SG | 362 |
| ATOM | 362 | O | LEU | 44 | −3.390 | 14.831 | −0.600 | 1.00 | 0.66 | 1SG | 363 |
| ATOM | 363 | N | GLU | 45 | −1.327 | 15.067 | 0.291 | 1.00 | 0.69 | 1SG | 364 |
| ATOM | 364 | CA | GLU | 45 | −1.374 | 16.497 | 0.224 | 1.00 | 0.69 | 1SG | 365 |
| ATOM | 365 | CB | GLU | 45 | −0.977 | 17.181 | 1.545 | 1.00 | 0.69 | 1SG | 366 |
| ATOM | 366 | CG | GLU | 45 | −1.888 | 16.845 | 2.724 | 1.00 | 0.69 | 1SG | 367 |
| ATOM | 367 | CD | GLU | 45 | −1.292 | 17.498 | 3.964 | 1.00 | 0.69 | 1SG | 368 |
| ATOM | 368 | OE1 | GLU | 45 | −0.539 | 18.496 | 3.805 | 1.00 | 0.69 | 1SG | 369 |
| ATOM | 369 | OE2 | GLU | 45 | −1.596 | 17.014 | 5.086 | 1.00 | 0.69 | 1SG | 370 |
| ATOM | 370 | C | GLU | 45 | −0.306 | 16.877 | −0.753 | 1.00 | 0.69 | 1SG | 371 |
| ATOM | 371 | O | GLU | 45 | 0.506 | 16.040 | −1.139 | 1.00 | 0.69 | 1SG | 372 |
| ATOM | 372 | N | ILE | 46 | −0.301 | 18.152 | −1.190 | 1.00 | 0.87 | 1SG | 373 |
| ATOM | 373 | CA | ILE | 46 | 0.722 | 18.695 | −2.049 | 1.00 | 0.87 | 1SG | 374 |
| ATOM | 374 | CB | ILE | 46 | 2.106 | 18.399 | −1.583 | 1.00 | 0.87 | 1SG | 375 |
| ATOM | 375 | CG2 | ILE | 46 | 3.091 | 18.989 | −2.600 | 1.00 | 0.87 | 1SG | 376 |
| ATOM | 376 | CG1 | ILE | 46 | 2.293 | 19.000 | −0.192 | 1.00 | 0.87 | 1SG | 377 |
| ATOM | 377 | CD1 | ILE | 46 | 2.146 | 20.517 | −0.128 | 1.00 | 0.87 | 1SG | 378 |
| ATOM | 378 | C | ILE | 46 | 0.596 | 18.237 | −3.473 | 1.00 | 0.87 | 1SG | 379 |
| ATOM | 379 | O | ILE | 46 | 1.021 | 18.939 | −4.388 | 1.00 | 0.87 | 1SG | 380 |
| ATOM | 380 | N | ARG | 47 | −0.012 | 17.067 | −3.725 | 1.00 | 1.20 | 1SG | 381 |
| ATOM | 381 | CA | ARG | 47 | −0.155 | 16.633 | −5.087 | 1.00 | 1.20 | 1SG | 382 |
| ATOM | 382 | CB | ARG | 47 | −0.973 | 17.605 | −5.964 | 1.00 | 1.20 | 1SG | 383 |
| ATOM | 383 | CG | ARG | 47 | −1.274 | 17.066 | −7.368 | 1.00 | 1.20 | 1SG | 384 |
| ATOM | 384 | CD | ARG | 47 | −2.022 | 18.049 | −8.277 | 1.00 | 1.20 | 1SG | 385 |
| ATOM | 385 | NE | ARG | 47 | −1.011 | 18.785 | −9.091 | 1.00 | 1.20 | 1SG | 386 |
| ATOM | 386 | CZ | ARG | 47 | −1.319 | 19.203 | −10.355 | 1.00 | 1.20 | 1SG | 387 |
| ATOM | 387 | NH1 | ARG | 47 | −2.555 | 18.957 | −10.881 | 1.00 | 1.20 | 1SG | 388 |
| ATOM | 388 | NH2 | ARG | 47 | −0.385 | 19.865 | −11.099 | 1.00 | 1.20 | 1SG | 389 |
| ATOM | 389 | C | ARG | 47 | 1.190 | 16.460 | −5.720 | 1.00 | 1.20 | 1SG | 390 |
| ATOM | 390 | O | ARG | 47 | 1.412 | 16.883 | −6.854 | 1.00 | 1.20 | 1SG | 391 |
| ATOM | 391 | N | ARG | 48 | 2.144 | 15.828 | −5.011 | 1.00 | 1.22 | 1SG | 392 |
| ATOM | 392 | CA | ARG | 48 | 3.375 | 15.585 | −5.692 | 1.00 | 1.22 | 1SG | 393 |
| ATOM | 393 | CB | ARG | 48 | 4.605 | 15.475 | −4.783 | 1.00 | 1.22 | 1SG | 394 |
| ATOM | 394 | CG | ARG | 48 | 5.129 | 16.822 | −4.283 | 1.00 | 1.22 | 1SG | 395 |
| ATOM | 395 | CD | ARG | 48 | 6.311 | 17.351 | −5.102 | 1.00 | 1.22 | 1SG | 396 |
| ATOM | 396 | NE | ARG | 48 | 5.782 | 17.769 | −6.430 | 1.00 | 1.22 | 1SG | 397 |
| ATOM | 397 | CZ | ARG | 48 | 5.338 | 19.046 | −6.615 | 1.00 | 1.22 | 1SG | 398 |
| ATOM | 398 | NH1 | ARG | 48 | 5.435 | 19.957 | −5.603 | 1.00 | 1.22 | 1SG | 399 |
| ATOM | 399 | NH2 | ARG | 48 | 4.806 | 19.412 | −7.817 | 1.00 | 1.22 | 1SG | 400 |
| ATOM | 400 | C | ARG | 48 | 3.166 | 14.310 | −6.436 | 1.00 | 1.22 | 1SG | 401 |
| ATOM | 401 | O | ARG | 48 | 2.775 | 13.290 | −5.869 | 1.00 | 1.22 | 1SG | 402 |
| ATOM | 402 | N | ARG | 49 | 3.438 | 14.360 | −7.751 | 1.00 | 0.88 | 1SG | 403 |
| ATOM | 403 | CA | ARG | 49 | 3.165 | 13.285 | −8.653 | 1.00 | 0.88 | 1SG | 404 |
| ATOM | 404 | CB | ARG | 49 | 3.593 | 13.612 | −10.095 | 1.00 | 0.88 | 1SG | 405 |
| ATOM | 405 | CG | ARG | 49 | 2.752 | 14.723 | −10.729 | 1.00 | 0.88 | 1SG | 406 |
| ATOM | 406 | CD | ARG | 49 | 2.098 | 14.329 | −12.055 | 1.00 | 0.88 | 1SG | 407 |
| ATOM | 407 | NE | ARG | 49 | 3.150 | 14.374 | −13.111 | 1.00 | 0.88 | 1SG | 408 |
| ATOM | 408 | CZ | ARG | 49 | 3.143 | 13.465 | −14.130 | 1.00 | 0.88 | 1SG | 409 |
| ATOM | 409 | NH1 | ARG | 49 | 2.211 | 12.468 | −14.151 | 1.00 | 0.88 | 1SG | 410 |
| ATOM | 410 | NH2 | ARG | 49 | 4.069 | 13.553 | −15.128 | 1.00 | 0.88 | 1SG | 411 |
| ATOM | 411 | C | ARG | 49 | 3.901 | 12.066 | −8.221 | 1.00 | 0.88 | 1SG | 412 |
| ATOM | 412 | O | ARG | 49 | 3.370 | 10.960 | −8.313 | 1.00 | 0.88 | 1SG | 413 |
| ATOM | 413 | N | ASP | 50 | 5.137 | 12.236 | −7.721 | 1.00 | 0.51 | 1SG | 414 |
| ATOM | 414 | CA | ASP | 50 | 5.927 | 11.091 | −7.387 | 1.00 | 0.51 | 1SG | 415 |
| ATOM | 415 | CB | ASP | 50 | 7.272 | 11.492 | −6.753 | 1.00 | 0.51 | 1SG | 416 |
| ATOM | 416 | CG | ASP | 50 | 8.105 | 12.223 | −7.803 | 1.00 | 0.51 | 1SG | 417 |
| ATOM | 417 | OD1 | ASP | 50 | 8.027 | 11.840 | −9.001 | 1.00 | 0.51 | 1SG | 418 |
| ATOM | 418 | OD2 | ASP | 50 | 8.823 | 13.185 | −7.421 | 1.00 | 0.51 | 1SG | 419 |
| ATOM | 419 | C | ASP | 50 | 5.183 | 10.246 | −6.394 | 1.00 | 0.51 | 1SG | 420 |
| ATOM | 420 | O | ASP | 50 | 4.833 | 9.106 | −6.693 | 1.00 | 0.51 | 1SG | 421 |
| ATOM | 421 | N | ILE | 51 | 4.818 | 10.806 | −5.224 | 1.00 | 0.50 | 1SG | 422 |
| ATOM | 422 | CA | ILE | 51 | 4.200 | 9.984 | −4.219 | 1.00 | 0.50 | 1SG | 423 |
| ATOM | 423 | CB | ILE | 51 | 3.824 | 10.701 | −2.949 | 1.00 | 0.50 | 1SG | 424 |
| ATOM | 424 | CG2 | ILE | 51 | 2.830 | 11.839 | −3.234 | 1.00 | 0.50 | 1SG | 425 |
| ATOM | 425 | CG1 | ILE | 51 | 3.328 | 9.704 | −1.898 | 1.00 | 0.50 | 1SG | 426 |
| ATOM | 426 | CD1 | ILE | 51 | 2.965 | 10.399 | −0.587 | 1.00 | 0.50 | 1SG | 427 |
| ATOM | 427 | C | ILE | 51 | 2.926 | 9.419 | −4.731 | 1.00 | 0.50 | 1SG | 428 |
| ATOM | 428 | O | ILE | 51 | 2.628 | 8.249 | −4.507 | 1.00 | 0.50 | 1SG | 429 |
| ATOM | 429 | N | ILE | 52 | 2.117 | 10.232 | −5.418 | 1.00 | 0.57 | 1SG | 430 |
| ATOM | 430 | CA | ILE | 52 | 0.839 | 9.719 | −5.776 | 1.00 | 0.57 | 1SG | 431 |
| ATOM | 431 | CB | ILE | 52 | −0.039 | 10.792 | −6.360 | 1.00 | 0.57 | 1SG | 432 |
| ATOM | 432 | CG2 | ILE | 52 | 0.719 | 11.514 | −7.487 | 1.00 | 0.57 | 1SG | 433 |
| ATOM | 433 | CG1 | ILE | 52 | −1.409 | 10.216 | −6.740 | 1.00 | 0.57 | 1SG | 434 |
| ATOM | 434 | CD1 | ILE | 52 | −2.441 | 11.288 | −7.083 | 1.00 | 0.57 | 1SG | 435 |
| ATOM | 435 | C | ILE | 52 | 0.945 | 8.583 | −6.739 | 1.00 | 0.57 | 1SG | 436 |
| ATOM | 436 | O | ILE | 52 | 0.562 | 7.457 | −6.423 | 1.00 | 0.57 | 1SG | 437 |
| ATOM | 437 | N | ASP | 53 | 1.506 | 8.849 | −7.931 | 1.00 | 0.64 | 1SG | 438 |
| ATOM | 438 | CA | ASP | 53 | 1.542 | 7.876 | −8.978 | 1.00 | 0.64 | 1SG | 439 |
| ATOM | 439 | CB | ASP | 53 | 1.863 | 8.504 | −10.346 | 1.00 | 0.64 | 1SG | 440 |
| ATOM | 440 | CG | ASP | 53 | 1.578 | 7.480 | −11.439 | 1.00 | 0.64 | 1SG | 441 |
| ATOM | 441 | OD1 | ASP | 53 | 2.155 | 6.360 | −11.389 | 1.00 | 0.64 | 1SG | 442 |
| ATOM | 442 | OD2 | ASP | 53 | 0.777 | 7.815 | −12.352 | 1.00 | 0.64 | 1SG | 443 |
| ATOM | 443 | C | ASP | 53 | 2.575 | 6.842 | −8.725 | 1.00 | 0.64 | 1SG | 444 |
| ATOM | 444 | O | ASP | 53 | 2.322 | 5.646 | −8.871 | 1.00 | 0.64 | 1SG | 445 |
| ATOM | 445 | N | LEU | 54 | 3.764 | 7.288 | −8.291 | 1.00 | 0.83 | 1SG | 446 |
| ATOM | 446 | CA | LEU | 54 | 4.873 | 6.392 | −8.270 | 1.00 | 0.83 | 1SG | 447 |
| ATOM | 447 | CB | LEU | 54 | 6.205 | 7.070 | −7.908 | 1.00 | 0.83 | 1SG | 448 |
| ATOM | 448 | CG | LEU | 54 | 7.474 | 6.208 | −8.073 | 1.00 | 0.83 | 1SG | 449 |
| ATOM | 449 | CD2 | LEU | 54 | 7.664 | 5.790 | −9.541 | 1.00 | 0.83 | 1SG | 450 |
| ATOM | 450 | CD1 | LEU | 54 | 7.527 | 5.034 | −7.090 | 1.00 | 0.83 | 1SG | 451 |
| ATOM | 451 | C | LEU | 54 | 4.582 | 5.266 | −7.367 | 1.00 | 0.83 | 1SG | 452 |
| ATOM | 452 | O | LEU | 54 | 4.997 | 4.146 | −7.656 | 1.00 | 0.83 | 1SG | 453 |
| ATOM | 453 | N | GLU | 55 | 3.857 | 5.540 | −6.263 | 1.00 | 0.90 | 1SG | 454 |
| ATOM | 454 | CA | GLU | 55 | 3.465 | 4.518 | −5.336 | 1.00 | 0.90 | 1SG | 455 |
| ATOM | 455 | CB | GLU | 55 | 3.165 | 3.146 | −5.976 | 1.00 | 0.90 | 1SG | 456 |
| ATOM | 456 | CG | GLU | 55 | 2.419 | 3.274 | −7.307 | 1.00 | 0.90 | 1SG | 457 |
| ATOM | 457 | CD | GLU | 55 | 2.627 | 2.030 | −8.162 | 1.00 | 0.90 | 1SG | 458 |
| ATOM | 458 | OE1 | GLU | 55 | 3.656 | 1.333 | −7.960 | 1.00 | 0.90 | 1SG | 459 |
| ATOM | 459 | OE2 | GLU | 55 | 1.756 | 1.763 | −9.032 | 1.00 | 0.90 | 1SG | 460 |
| ATOM | 460 | C | GLU | 55 | 4.657 | 4.310 | −4.483 | 1.00 | 0.90 | 1SG | 461 |
| ATOM | 461 | O | GLU | 55 | 4.607 | 4.457 | −3.264 | 1.00 | 0.90 | 1SG | 462 |
| ATOM | 462 | N | ASP | 56 | 5.780 | 3.997 | −5.150 | 1.00 | 0.87 | 1SG | 463 |
| ATOM | 463 | CA | ASP | 56 | 7.012 | 3.700 | −4.500 | 1.00 | 0.87 | 1SG | 464 |
| ATOM | 464 | CB | ASP | 56 | 7.340 | 4.586 | −3.286 | 1.00 | 0.87 | 1SG | 465 |
| ATOM | 465 | CG | ASP | 56 | 8.735 | 4.208 | −2.813 | 1.00 | 0.87 | 1SG | 466 |
| ATOM | 466 | OD1 | ASP | 56 | 9.310 | 3.244 | −3.385 | 1.00 | 0.87 | 1SG | 467 |
| ATOM | 467 | OD2 | ASP | 56 | 9.248 | 4.881 | −1.880 | 1.00 | 0.87 | 1SG | 468 |
| ATOM | 468 | C | ASP | 56 | 6.813 | 2.311 | −4.044 | 1.00 | 0.87 | 1SG | 469 |
| ATOM | 469 | O | ASP | 56 | 6.743 | 1.392 | −4.858 | 1.00 | 0.87 | 1SG | 470 |
| ATOM | 470 | N | ASP | 57 | 6.712 | 2.110 | −2.722 | 1.00 | 0.74 | 1SG | 471 |
| ATOM | 471 | CA | ASP | 57 | 6.418 | 0.773 | −2.340 | 1.00 | 0.74 | 1SG | 472 |
| ATOM | 472 | CB | ASP | 57 | 6.381 | 0.555 | −0.821 | 1.00 | 0.74 | 1SG | 473 |
| ATOM | 473 | CG | ASP | 57 | 6.375 | −0.951 | −0.585 | 1.00 | 0.74 | 1SG | 474 |
| ATOM | 474 | OD1 | ASP | 57 | 5.717 | −1.683 | −1.372 | 1.00 | 0.74 | 1SG | 475 |
| ATOM | 475 | OD2 | ASP | 57 | 7.049 | −1.393 | 0.382 | 1.00 | 0.74 | 1SG | 476 |
| ATOM | 476 | C | ASP | 57 | 5.056 | 0.504 | −2.885 | 1.00 | 0.74 | 1SG | 477 |
| ATOM | 477 | O | ASP | 57 | 4.890 | −0.402 | −3.700 | 1.00 | 0.74 | 1SG | 478 |
| ATOM | 478 | N | ASP | 58 | 4.058 | 1.327 | −2.483 | 1.00 | 0.51 | 1SG | 479 |
| ATOM | 479 | CA | ASP | 58 | 2.730 | 1.114 | −2.985 | 1.00 | 0.51 | 1SG | 480 |
| ATOM | 480 | CB | ASP | 58 | 2.020 | −0.086 | −2.326 | 1.00 | 0.51 | 1SG | 481 |
| ATOM | 481 | CG | ASP | 58 | 2.496 | −1.426 | −2.871 | 1.00 | 0.51 | 1SG | 482 |
| ATOM | 482 | OD1 | ASP | 58 | 2.137 | −1.761 | −4.031 | 1.00 | 0.51 | 1SG | 483 |
| ATOM | 483 | OD2 | ASP | 58 | 3.205 | −2.147 | −2.119 | 1.00 | 0.51 | 1SG | 484 |
| ATOM | 484 | C | ASP | 58 | 1.835 | 2.280 | −2.656 | 1.00 | 0.51 | 1SG | 485 |
| ATOM | 485 | O | ASP | 58 | 0.800 | 2.053 | −2.029 | 1.00 | 0.51 | 1SG | 486 |
| ATOM | 486 | N | VAL | 59 | 2.163 | 3.542 | −3.035 | 1.00 | 0.45 | 1SG | 487 |
| ATOM | 487 | CA | VAL | 59 | 1.179 | 4.546 | −2.733 | 1.00 | 0.45 | 1SG | 488 |
| ATOM | 488 | CB | VAL | 59 | 1.590 | 5.931 | −3.142 | 1.00 | 0.45 | 1SG | 489 |
| ATOM | 489 | CG1 | VAL | 59 | 0.389 | 6.871 | −2.940 | 1.00 | 0.45 | 1SG | 490 |
| ATOM | 490 | CG2 | VAL | 59 | 2.831 | 6.337 | −2.328 | 1.00 | 0.45 | 1SG | 491 |
| ATOM | 491 | C | VAL | 59 | −0.033 | 4.181 | −3.526 | 1.00 | 0.45 | 1SG | 492 |
| ATOM | 492 | O | VAL | 59 | −1.021 | 3.699 | −2.976 | 1.00 | 0.45 | 1SG | 493 |
| ATOM | 493 | N | VAL | 60 | 0.027 | 4.355 | −4.860 | 1.00 | 0.41 | 1SG | 494 |
| ATOM | 494 | CA | VAL | 60 | −1.056 | 3.892 | −5.675 | 1.00 | 0.41 | 1SG | 495 |
| ATOM | 495 | CB | VAL | 60 | −1.313 | 4.666 | −6.947 | 1.00 | 0.41 | 1SG | 496 |
| ATOM | 496 | CG1 | VAL | 60 | −0.141 | 4.550 | −7.920 | 1.00 | 0.41 | 1SG | 497 |
| ATOM | 497 | CG2 | VAL | 60 | −2.643 | 4.167 | −7.538 | 1.00 | 0.41 | 1SG | 498 |
| ATOM | 498 | C | VAL | 60 | −0.829 | 2.438 | −5.948 | 1.00 | 0.41 | 1SG | 499 |
| ATOM | 499 | O | VAL | 60 | −1.753 | 1.705 | −6.296 | 1.00 | 0.41 | 1SG | 500 |
| ATOM | 500 | N | ALA | 61 | 0.435 | 1.993 | −5.785 | 1.00 | 0.42 | 1SG | 501 |
| ATOM | 501 | CA | ALA | 61 | 0.836 | 0.646 | −6.080 | 1.00 | 0.42 | 1SG | 502 |
| ATOM | 502 | CB | ALA | 61 | 2.311 | 0.334 | −5.787 | 1.00 | 0.42 | 1SG | 503 |
| ATOM | 503 | C | ALA | 61 | 0.045 | −0.281 | −5.232 | 1.00 | 0.42 | 1SG | 504 |
| ATOM | 504 | O | ALA | 61 | −0.287 | −1.379 | −5.677 | 1.00 | 0.42 | 1SG | 505 |
| ATOM | 505 | N | MET | 62 | −0.257 | 0.121 | −3.981 | 1.00 | 0.51 | 1SG | 506 |
| ATOM | 506 | CA | MET | 62 | −1.061 | −0.748 | −3.173 | 1.00 | 0.51 | 1SG | 507 |
| ATOM | 507 | CB | MET | 62 | −1.466 | −0.176 | −1.803 | 1.00 | 0.51 | 1SG | 508 |
| ATOM | 508 | CG | MET | 62 | −0.419 | −0.359 | −0.707 | 1.00 | 0.51 | 1SG | 509 |
| ATOM | 509 | SD | MET | 62 | −0.080 | −2.101 | −0.305 | 1.00 | 0.51 | 1SG | 510 |
| ATOM | 510 | CE | MET | 62 | 1.086 | −1.737 | 1.038 | 1.00 | 0.51 | 1SG | 511 |
| ATOM | 511 | C | MET | 62 | −2.334 | −0.947 | −3.920 | 1.00 | 0.51 | 1SG | 512 |
| ATOM | 512 | O | MET | 62 | −2.858 | −2.058 | −3.992 | 1.00 | 0.51 | 1SG | 513 |
| ATOM | 513 | N | THR | 63 | −2.857 | 0.144 | −4.501 | 1.00 | 0.38 | 1SG | 514 |
| ATOM | 514 | CA | THR | 63 | −4.040 | 0.034 | −5.294 | 1.00 | 0.38 | 1SG | 515 |
| ATOM | 515 | CB | THR | 63 | −3.970 | −1.066 | −6.315 | 1.00 | 0.38 | 1SG | 516 |
| ATOM | 516 | OG1 | THR | 63 | −2.868 | −0.855 | −7.187 | 1.00 | 0.38 | 1SG | 517 |
| ATOM | 517 | CG2 | THR | 63 | −5.277 | −1.066 | −7.130 | 1.00 | 0.38 | 1SG | 518 |
| ATOM | 518 | C | THR | 63 | −5.181 | −0.228 | −4.376 | 1.00 | 0.38 | 1SG | 519 |
| ATOM | 519 | O | THR | 63 | −5.655 | 0.669 | −3.682 | 1.00 | 0.38 | 1SG | 520 |
| ATOM | 520 | N | SER | 64 | −5.656 | −1.484 | −4.369 | 1.00 | 0.31 | 1SG | 521 |
| ATOM | 521 | CA | SER | 64 | −6.777 | −1.875 | −3.571 | 1.00 | 0.31 | 1SG | 522 |
| ATOM | 522 | CB | SER | 64 | −7.287 | −3.284 | −3.905 | 1.00 | 0.31 | 1SG | 523 |
| ATOM | 523 | OG | SER | 64 | −6.272 | −4.241 | −3.646 | 1.00 | 0.31 | 1SG | 524 |
| ATOM | 524 | C | SER | 64 | −6.435 | −1.846 | −2.117 | 1.00 | 0.31 | 1SG | 525 |
| ATOM | 525 | O | SER | 64 | −7.299 | −1.571 | −1.286 | 1.00 | 0.31 | 1SG | 526 |
| ATOM | 526 | N | ASP | 65 | −5.167 | −2.123 | −1.759 | 1.00 | 0.45 | 1SG | 527 |
| ATOM | 527 | CA | ASP | 65 | −4.846 | −2.247 | −0.365 | 1.00 | 0.45 | 1SG | 528 |
| ATOM | 528 | CB | ASP | 65 | −3.378 | −2.633 | −0.109 | 1.00 | 0.45 | 1SG | 529 |
| ATOM | 529 | CG | ASP | 65 | −3.227 | −2.827 | 1.393 | 1.00 | 0.45 | 1SG | 530 |
| ATOM | 530 | OD1 | ASP | 65 | −3.470 | −3.967 | 1.872 | 1.00 | 0.45 | 1SG | 531 |
| ATOM | 531 | OD2 | ASP | 65 | −2.897 | −1.825 | 2.083 | 1.00 | 0.45 | 1SG | 532 |
| ATOM | 532 | C | ASP | 65 | −5.160 | −0.969 | 0.353 | 1.00 | 0.45 | 1SG | 533 |
| ATOM | 533 | O | ASP | 65 | −5.702 | −1.002 | 1.458 | 1.00 | 0.45 | 1SG | 534 |
| ATOM | 534 | N | CYS | 66 | −4.843 | 0.194 | −0.248 | 1.00 | 0.48 | 1SG | 535 |
| ATOM | 535 | CA | CYS | 66 | −5.116 | 1.426 | 0.437 | 1.00 | 0.48 | 1SG | 536 |
| ATOM | 536 | CB | CYS | 66 | −3.860 | 2.283 | 0.638 | 1.00 | 0.48 | 1SG | 537 |
| ATOM | 537 | SG | CYS | 66 | −3.160 | 2.790 | −0.961 | 1.00 | 0.48 | 1SG | 538 |
| ATOM | 538 | C | CYS | 66 | −6.039 | 2.249 | −0.405 | 1.00 | 0.48 | 1SG | 539 |
| ATOM | 539 | O | CYS | 66 | −6.165 | 2.033 | −1.608 | 1.00 | 0.48 | 1SG | 540 |
| ATOM | 540 | N | ASP | 67 | −6.745 | 3.214 | 0.218 | 1.00 | 0.47 | 1SG | 541 |
| ATOM | 541 | CA | ASP | 67 | −7.571 | 4.081 | −0.565 | 1.00 | 0.47 | 1SG | 542 |
| ATOM | 542 | CB | ASP | 67 | −9.006 | 4.240 | −0.041 | 1.00 | 0.47 | 1SG | 543 |
| ATOM | 543 | CG | ASP | 67 | −9.829 | 4.849 | −1.171 | 1.00 | 0.47 | 1SG | 544 |
| ATOM | 544 | OD1 | ASP | 67 | −9.245 | 5.104 | −2.259 | 1.00 | 0.47 | 1SG | 545 |
| ATOM | 545 | OD2 | ASP | 67 | −11.053 | 5.065 | −0.963 | 1.00 | 0.47 | 1SG | 546 |
| ATOM | 546 | C | ASP | 67 | −6.916 | 5.419 | −0.503 | 1.00 | 0.47 | 1SG | 547 |
| ATOM | 547 | O | ASP | 67 | −6.732 | 5.981 | 0.575 | 1.00 | 0.47 | 1SG | 548 |
| ATOM | 548 | N | ILE | 68 | −6.537 | 5.972 | −1.669 | 1.00 | 0.61 | 1SG | 549 |
| ATOM | 549 | CA | ILE | 68 | −5.822 | 7.209 | −1.631 | 1.00 | 0.61 | 1SG | 550 |
| ATOM | 550 | CB | ILE | 68 | −4.620 | 7.220 | −2.526 | 1.00 | 0.61 | 1SG | 551 |
| ATOM | 551 | CG2 | ILE | 68 | −3.631 | 6.168 | −2.007 | 1.00 | 0.61 | 1SG | 552 |
| ATOM | 552 | CG1 | ILE | 68 | −5.044 | 7.025 | −3.992 | 1.00 | 0.61 | 1SG | 553 |
| ATOM | 553 | CD1 | ILE | 68 | −3.912 | 7.279 | −4.987 | 1.00 | 0.61 | 1SG | 554 |
| ATOM | 554 | C | ILE | 68 | −6.709 | 8.307 | −2.089 | 1.00 | 0.61 | 1SG | 555 |
| ATOM | 555 | O | ILE | 68 | −7.357 | 8.223 | −3.133 | 1.00 | 0.61 | 1SG | 556 |
| ATOM | 556 | N | TYR | 69 | −6.777 | 9.381 | −1.287 | 1.00 | 0.65 | 1SG | 557 |
| ATOM | 557 | CA | TYR | 69 | −7.549 | 10.452 | −1.799 | 1.00 | 0.65 | 1SG | 558 |
| ATOM | 558 | CB | TYR | 69 | −8.899 | 10.702 | −1.105 | 1.00 | 0.65 | 1SG | 559 |
| ATOM | 559 | CG | TYR | 69 | −9.661 | 11.386 | −2.198 | 1.00 | 0.65 | 1SG | 560 |
| ATOM | 560 | CD1 | TYR | 69 | −10.223 | 10.626 | −3.201 | 1.00 | 0.65 | 1SG | 561 |
| ATOM | 561 | CD2 | TYR | 69 | −9.801 | 12.753 | −2.261 | 1.00 | 0.65 | 1SG | 562 |
| ATOM | 562 | CE1 | TYR | 69 | −10.914 | 11.201 | −4.240 | 1.00 | 0.65 | 1SG | 563 |
| ATOM | 563 | CE2 | TYR | 69 | −10.486 | 13.342 | −3.300 | 1.00 | 0.65 | 1SG | 564 |
| ATOM | 564 | CZ | TYR | 69 | −11.041 | 12.565 | −4.292 | 1.00 | 0.65 | 1SG | 565 |
| ATOM | 565 | OH | TYR | 69 | −11.746 | 13.144 | −5.365 | 1.00 | 0.65 | 1SG | 566 |
| ATOM | 566 | C | TYR | 69 | −6.660 | 11.651 | −1.763 | 1.00 | 0.65 | 1SG | 567 |
| ATOM | 567 | O | TYR | 69 | −5.638 | 11.664 | −1.077 | 1.00 | 0.65 | 1SG | 568 |
| ATOM | 568 | N | GLN | 70 | −7.024 | 12.676 | −2.547 | 1.00 | 0.55 | 1SG | 569 |
| ATOM | 569 | CA | GLN | 70 | −6.244 | 13.856 | −2.744 | 1.00 | 0.55 | 1SG | 570 |
| ATOM | 570 | CB | GLN | 70 | −6.343 | 14.375 | −4.182 | 1.00 | 0.55 | 1SG | 571 |
| ATOM | 571 | CG | GLN | 70 | −6.093 | 13.329 | −5.261 | 1.00 | 0.55 | 1SG | 572 |
| ATOM | 572 | CD | GLN | 70 | −6.687 | 13.916 | −6.528 | 1.00 | 0.55 | 1SG | 573 |
| ATOM | 573 | OE1 | GLN | 70 | −7.446 | 13.266 | −7.246 | 1.00 | 0.55 | 1SG | 574 |
| ATOM | 574 | NE2 | GLN | 70 | −6.345 | 15.203 | −6.796 | 1.00 | 0.55 | 1SG | 575 |
| ATOM | 575 | C | GLN | 70 | −6.877 | 14.964 | −1.987 | 1.00 | 0.55 | 1SG | 576 |
| ATOM | 576 | O | GLN | 70 | −8.096 | 15.131 | −2.002 | 1.00 | 0.55 | 1SG | 577 |
| ATOM | 577 | N | THR | 71 | −6.052 | 15.768 | −1.299 | 1.00 | 0.55 | 1SG | 578 |
| ATOM | 578 | CA | THR | 71 | −6.633 | 16.925 | −0.707 | 1.00 | 0.55 | 1SG | 579 |
| ATOM | 579 | CB | THR | 71 | −5.732 | 17.555 | 0.279 | 1.00 | 0.55 | 1SG | 580 |
| ATOM | 580 | OG1 | THR | 71 | −4.533 | 17.986 | −0.347 | 1.00 | 0.55 | 1SG | 581 |
| ATOM | 581 | CG2 | THR | 71 | −5.415 | 16.487 | 1.336 | 1.00 | 0.55 | 1SG | 582 |
| ATOM | 582 | C | THR | 71 | −6.863 | 17.861 | −1.847 | 1.00 | 0.55 | 1SG | 583 |
| ATOM | 583 | O | THR | 71 | −5.957 | 18.112 | −2.639 | 1.00 | 0.55 | 1SG | 584 |
| ATOM | 584 | N | LEU | 72 | −8.103 | 18.376 | −1.958 | 1.00 | 0.56 | 1SG | 585 |
| ATOM | 585 | CA | LEU | 72 | −8.535 | 19.206 | −3.047 | 1.00 | 0.56 | 1SG | 586 |
| ATOM | 586 | CB | LEU | 72 | −10.048 | 19.497 | −2.983 | 1.00 | 0.56 | 1SG | 587 |
| ATOM | 587 | CG | LEU | 72 | −10.947 | 18.249 | −3.140 | 1.00 | 0.56 | 1SG | 588 |
| ATOM | 588 | CD2 | LEU | 72 | −10.704 | 17.222 | −2.019 | 1.00 | 0.56 | 1SG | 589 |
| ATOM | 589 | CD1 | LEU | 72 | −10.826 | 17.639 | −4.546 | 1.00 | 0.56 | 1SG | 590 |
| ATOM | 590 | C | LEU | 72 | −7.808 | 20.525 | −3.065 | 1.00 | 0.56 | 1SG | 591 |
| ATOM | 591 | O | LEU | 72 | −7.431 | 21.009 | −4.130 | 1.00 | 0.56 | 1SG | 592 |
| ATOM | 592 | N | ARG | 73 | −7.575 | 21.132 | −1.884 | 1.00 | 0.45 | 1SG | 593 |
| ATOM | 593 | CA | ARG | 73 | −7.028 | 22.461 | −1.779 | 1.00 | 0.45 | 1SG | 594 |
| ATOM | 594 | CB | ARG | 73 | −7.256 | 23.059 | −0.391 | 1.00 | 0.45 | 1SG | 595 |
| ATOM | 595 | CG | ARG | 73 | −8.730 | 23.180 | −0.033 | 1.00 | 0.45 | 1SG | 596 |
| ATOM | 596 | CD | ARG | 73 | −8.937 | 23.217 | 1.476 | 1.00 | 0.45 | 1SG | 597 |
| ATOM | 597 | NE | ARG | 73 | −10.387 | 23.447 | 1.715 | 1.00 | 0.45 | 1SG | 598 |
| ATOM | 598 | CZ | ARG | 73 | −11.267 | 22.407 | 1.615 | 1.00 | 0.45 | 1SG | 599 |
| ATOM | 599 | NH1 | ARG | 73 | −10.855 | 21.197 | 1.138 | 1.00 | 0.45 | 1SG | 600 |
| ATOM | 600 | NH2 | ARG | 73 | −12.566 | 22.589 | 1.998 | 1.00 | 0.45 | 1SG | 601 |
| ATOM | 601 | C | ARG | 73 | −5.543 | 22.461 | −1.953 | 1.00 | 0.45 | 1SG | 602 |
| ATOM | 602 | O | ARG | 73 | −4.799 | 22.093 | −1.046 | 1.00 | 0.45 | 1SG | 603 |
| ATOM | 603 | N | GLY | 74 | −5.080 | 22.900 | −3.138 | 1.00 | 0.41 | 1SG | 604 |
| ATOM | 604 | CA | GLY | 74 | −3.682 | 23.035 | −3.434 | 1.00 | 0.41 | 1SG | 605 |
| ATOM | 605 | C | GLY | 74 | −3.162 | 24.234 | −2.716 | 1.00 | 0.41 | 1SG | 606 |
| ATOM | 606 | O | GLY | 74 | −1.983 | 24.297 | −2.363 | 1.00 | 0.41 | 1SG | 607 |
| ATOM | 607 | N | TYR | 75 | −4.067 | 25.223 | −2.529 | 1.00 | 0.45 | 1SG | 608 |
| ATOM | 608 | CA | TYR | 75 | −3.872 | 26.504 | −1.906 | 1.00 | 0.45 | 1SG | 609 |
| ATOM | 609 | CB | TYR | 75 | −5.040 | 26.952 | −1.003 | 1.00 | 0.45 | 1SG | 610 |
| ATOM | 610 | CG | TYR | 75 | −6.105 | 27.679 | −1.744 | 1.00 | 0.45 | 1SG | 611 |
| ATOM | 611 | CD1 | TYR | 75 | −5.937 | 29.030 | −1.944 | 1.00 | 0.45 | 1SG | 612 |
| ATOM | 612 | CD2 | TYR | 75 | −7.260 | 27.073 | −2.190 | 1.00 | 0.45 | 1SG | 613 |
| ATOM | 613 | CE1 | TYR | 75 | −6.880 | 29.777 | −2.606 | 1.00 | 0.45 | 1SG | 614 |
| ATOM | 614 | CE2 | TYR | 75 | −8.220 | 27.812 | −2.857 | 1.00 | 0.45 | 1SG | 615 |
| ATOM | 615 | CZ | TYR | 75 | −8.022 | 29.163 | −3.062 | 1.00 | 0.45 | 1SG | 616 |
| ATOM | 616 | OH | TYR | 75 | −8.998 | 29.925 | −3.741 | 1.00 | 0.45 | 1SG | 617 |
| ATOM | 617 | C | TYR | 75 | −2.715 | 26.532 | −0.977 | 1.00 | 0.45 | 1SG | 618 |
| ATOM | 618 | O | TYR | 75 | −2.524 | 25.623 | −0.173 | 1.00 | 0.45 | 1SG | 619 |
| ATOM | 619 | N | ALA | 76 | −1.883 | 27.586 | −1.092 | 1.00 | 0.49 | 1SG | 620 |
| ATOM | 620 | CA | ALA | 76 | −0.916 | 27.748 | −0.055 | 1.00 | 0.49 | 1SG | 621 |
| ATOM | 621 | CB | ALA | 76 | 0.428 | 28.361 | −0.482 | 1.00 | 0.49 | 1SG | 622 |
| ATOM | 622 | C | ALA | 76 | −1.610 | 28.708 | 0.832 | 1.00 | 0.49 | 1SG | 623 |
| ATOM | 623 | O | ALA | 76 | −2.219 | 29.659 | 0.345 | 1.00 | 0.49 | 1SG | 624 |
| ATOM | 624 | N | GLN | 77 | −1.570 | 28.486 | 2.155 | 1.00 | 0.75 | 1SG | 625 |
| ATOM | 625 | CA | GLN | 77 | −2.346 | 29.363 | 2.977 | 1.00 | 0.75 | 1SG | 626 |
| ATOM | 626 | CB | GLN | 77 | −2.507 | 28.848 | 4.419 | 1.00 | 0.75 | 1SG | 627 |
| ATOM | 627 | CG | GLN | 77 | −3.751 | 29.364 | 5.157 | 1.00 | 0.75 | 1SG | 628 |
| ATOM | 628 | CD | GLN | 77 | −3.602 | 30.830 | 5.547 | 1.00 | 0.75 | 1SG | 629 |
| ATOM | 629 | CE1 | GLN | 77 | −2.855 | 31.155 | 6.468 | 1.00 | 0.75 | 1SG | 630 |
| ATOM | 630 | NE2 | GLN | 77 | −4.374 | 31.731 | 4.879 | 1.00 | 0.75 | 1SG | 631 |
| ATOM | 631 | C | GLN | 77 | −1.639 | 30.679 | 2.986 | 1.00 | 0.75 | 1SG | 632 |
| ATOM | 632 | O | GLN | 77 | −0.409 | 30.744 | 2.982 | 1.00 | 0.75 | 1SG | 633 |
| ATOM | 633 | N | LYS | 78 | −2.417 | 31.781 | 2.969 | 1.00 | 1.06 | 1SG | 634 |
| ATOM | 634 | CA | LYS | 78 | −1.819 | 33.082 | 2.977 | 1.00 | 1.06 | 1SG | 635 |
| ATOM | 635 | CB | LYS | 78 | −2.807 | 34.261 | 3.006 | 1.00 | 1.06 | 1SG | 636 |
| ATOM | 636 | CG | LYS | 78 | −2.801 | 34.990 | 1.664 | 1.00 | 1.06 | 1SG | 637 |
| ATOM | 637 | CD | LYS | 78 | −1.411 | 35.557 | 1.355 | 1.00 | 1.06 | 1SG | 638 |
| ATOM | 638 | CE | LYS | 78 | −1.194 | 35.962 | −0.105 | 1.00 | 1.06 | 1SG | 639 |
| ATOM | 639 | NZ | LYS | 78 | −1.666 | 37.348 | −0.329 | 1.00 | 1.06 | 1SG | 640 |
| ATOM | 640 | C | LYS | 78 | −0.875 | 33.205 | 4.108 | 1.00 | 1.06 | 1SG | 641 |
| ATOM | 641 | O | LYS | 78 | 0.281 | 33.529 | 3.855 | 1.00 | 1.06 | 1SG | 642 |
| ATOM | 642 | N | LEU | 79 | −1.354 | 32.984 | 5.354 | 1.00 | 1.02 | 1SG | 643 |
| ATOM | 643 | CA | LEU | 79 | −0.541 | 32.980 | 6.537 | 1.00 | 1.02 | 1SG | 644 |
| ATOM | 644 | CB | LEU | 79 | 0.897 | 32.493 | 6.269 | 1.00 | 1.02 | 1SG | 645 |
| ATOM | 645 | CG | LEU | 79 | 1.930 | 32.637 | 7.374 | 1.00 | 1.02 | 1SG | 646 |
| ATOM | 646 | CD2 | LEU | 79 | 3.234 | 31.963 | 6.925 | 1.00 | 1.02 | 1SG | 647 |
| ATOM | 647 | CD1 | LEU | 79 | 1.420 | 32.030 | 8.668 | 1.00 | 1.02 | 1SG | 648 |
| ATOM | 648 | C | LEU | 79 | −0.591 | 34.340 | 7.135 | 1.00 | 1.02 | 1SG | 649 |
| ATOM | 649 | O | LEU | 79 | −0.865 | 35.323 | 6.448 | 1.00 | 1.02 | 1SG | 650 |
| ATOM | 650 | N | VAL | 80 | −0.353 | 34.414 | 8.456 | 1.00 | 0.70 | 1SG | 651 |
| ATOM | 651 | CA | VAL | 80 | −0.398 | 35.656 | 9.163 | 1.00 | 0.70 | 1SG | 652 |
| ATOM | 652 | CB | VAL | 80 | −0.567 | 35.458 | 10.626 | 1.00 | 0.70 | 1SG | 653 |
| ATOM | 653 | CG1 | VAL | 80 | −1.957 | 34.846 | 10.865 | 1.00 | 0.70 | 1SG | 654 |
| ATOM | 654 | CG2 | VAL | 80 | 0.581 | 34.552 | 11.085 | 1.00 | 0.70 | 1SG | 655 |
| ATOM | 655 | C | VAL | 80 | 0.893 | 36.355 | 8.916 | 1.00 | 0.70 | 1SG | 656 |
| ATOM | 656 | O | VAL | 80 | 1.836 | 35.769 | 8.395 | 1.00 | 0.70 | 1SG | 657 |
| ATOM | 657 | N | SER | 81 | 0.953 | 37.651 | 9.273 | 1.00 | 0.65 | 1SG | 658 |
| ATOM | 658 | CA | SER | 81 | 2.124 | 38.425 | 8.995 | 1.00 | 0.65 | 1SG | 659 |
| ATOM | 659 | CB | SER | 81 | 1.978 | 39.893 | 9.432 | 1.00 | 0.65 | 1SG | 660 |
| ATOM | 660 | OG | SER | 81 | 3.164 | 40.611 | 9.134 | 1.00 | 0.65 | 1SG | 661 |
| ATOM | 661 | C | SER | 81 | 3.307 | 37.872 | 9.726 | 1.00 | 0.65 | 1SG | 662 |
| ATOM | 662 | O | SER | 81 | 4.357 | 37.648 | 9.127 | 1.00 | 0.65 | 1SG | 663 |
| ATOM | 663 | N | LYS | 82 | 3.177 | 37.627 | 11.044 | 1.00 | 0.81 | 1SG | 664 |
| ATOM | 664 | CA | LYS | 82 | 4.323 | 37.200 | 11.800 | 1.00 | 0.81 | 1SG | 665 |
| ATOM | 665 | CB | LYS | 82 | 4.046 | 37.136 | 13.308 | 1.00 | 0.81 | 1SG | 666 |
| ATOM | 666 | CG | LYS | 82 | 5.308 | 36.907 | 14.143 | 1.00 | 0.81 | 1SG | 667 |
| ATOM | 667 | CD | LYS | 82 | 6.304 | 38.067 | 14.069 | 1.00 | 0.81 | 1SG | 668 |
| ATOM | 668 | CE | LYS | 82 | 5.717 | 39.417 | 14.488 | 1.00 | 0.81 | 1SG | 669 |
| ATOM | 669 | NZ | LYS | 82 | 5.701 | 39.538 | 15.964 | 1.00 | 0.81 | 1SG | 670 |
| ATOM | 670 | C | LYS | 82 | 4.764 | 35.841 | 11.357 | 1.00 | 0.81 | 1SG | 671 |
| ATOM | 671 | O | LYS | 82 | 5.943 | 35.605 | 11.099 | 1.00 | 0.81 | 1SG | 672 |
| ATOM | 672 | N | GLU | 83 | 3.798 | 34.918 | 11.242 | 1.00 | 0.61 | 1SG | 673 |
| ATOM | 673 | CA | GLU | 83 | 4.038 | 33.554 | 10.874 | 1.00 | 0.61 | 1SG | 674 |
| ATOM | 674 | CB | GLU | 83 | 2.832 | 32.648 | 11.167 | 1.00 | 0.61 | 1SG | 675 |
| ATOM | 675 | CG | GLU | 83 | 2.730 | 32.240 | 12.644 | 1.00 | 0.61 | 1SG | 676 |
| ATOM | 676 | CD | GLU | 83 | 2.743 | 33.482 | 13.535 | 1.00 | 0.61 | 1SG | 677 |
| ATOM | 677 | OE1 | GLU | 83 | 1.738 | 34.240 | 13.542 | 1.00 | 0.61 | 1SG | 678 |
| ATOM | 678 | OE2 | GLU | 83 | 3.777 | 33.689 | 14.225 | 1.00 | 0.61 | 1SG | 679 |
| ATOM | 679 | C | GLU | 83 | 4.428 | 33.505 | 9.430 | 1.00 | 0.61 | 1SG | 680 |
| ATOM | 680 | O | GLU | 83 | 4.991 | 32.526 | 8.947 | 1.00 | 0.61 | 1SG | 681 |
| ATOM | 681 | N | GLU | 84 | 4.153 | 34.594 | 8.702 | 1.00 | 0.46 | 1SG | 682 |
| ATOM | 682 | CA | GLU | 84 | 4.347 | 34.665 | 7.285 | 1.00 | 0.46 | 1SG | 683 |
| ATOM | 683 | CB | GLU | 84 | 4.094 | 36.084 | 6.739 | 1.00 | 0.46 | 1SG | 684 |
| ATOM | 684 | CG | GLU | 84 | 4.197 | 36.214 | 5.216 | 1.00 | 0.46 | 1SG | 685 |
| ATOM | 685 | CD | GLU | 84 | 3.829 | 3.646 | 4.849 | 1.00 | 0.46 | 1SG | 686 |
| ATOM | 686 | OE1 | GLU | 84 | 3.525 | 38.434 | 5.784 | 1.00 | 0.46 | 1SG | 687 |
| ATOM | 687 | OE2 | GLU | 84 | 3.844 | 37.971 | 3.632 | 1.00 | 0.46 | 1SG | 688 |
| ATOM | 688 | C | GLU | 84 | 5.750 | 34.280 | 6.938 | 1.00 | 0.46 | 1SG | 689 |
| ATOM | 689 | O | GLU | 84 | 5.971 | 33.646 | 5.907 | 1.00 | 0.46 | 1SG | 690 |
| ATOM | 690 | N | LYS | 85 | 6.735 | 34.632 | 7.786 | 1.00 | 0.70 | 1SG | 691 |
| ATOM | 691 | CA | LYS | 85 | 8.101 | 34.400 | 7.417 | 1.00 | 0.70 | 1SG | 692 |
| ATOM | 692 | CB | LYS | 85 | 9.098 | 34.787 | 8.524 | 1.00 | 0.70 | 1SG | 693 |
| ATOM | 693 | CG | LYS | 85 | 10.552 | 34.533 | 8.122 | 1.00 | 0.70 | 1SG | 694 |
| ATOM | 694 | CD | LYS | 85 | 11.579 | 35.189 | 9.048 | 1.00 | 0.70 | 1SG | 695 |
| ATOM | 695 | CE | LYS | 85 | 11.651 | 36.711 | 8.907 | 1.00 | 0.70 | 1SG | 696 |
| ATOM | 696 | NZ | LYS | 85 | 12.683 | 37.256 | 9.818 | 1.00 | 0.70 | 1SG | 697 |
| ATOM | 697 | C | LYS | 85 | 8.345 | 32.964 | 7.087 | 1.00 | 0.70 | 1SG | 698 |
| ATOM | 698 | O | LYS | 85 | 8.753 | 32.644 | 5.973 | 1.00 | 0.70 | 1SG | 699 |
| ATOM | 699 | N | SER | 86 | 8.082 | 32.023 | 8.006 | 1.00 | 0.77 | 1SG | 700 |
| ATOM | 700 | CA | SER | 86 | 8.385 | 30.700 | 7.550 | 1.00 | 0.77 | 1SG | 701 |
| ATOM | 701 | CB | SER | 86 | 9.790 | 30.221 | 7.944 | 1.00 | 0.77 | 1SG | 702 |
| ATOM | 702 | OG | SER | 86 | 10.778 | 31.042 | 7.339 | 1.00 | 0.77 | 1SG | 703 |
| ATOM | 703 | C | SER | 86 | 7.429 | 29.760 | 8.175 | 1.00 | 0.77 | 1SG | 704 |
| ATOM | 704 | O | SER | 86 | 7.763 | 29.089 | 9.151 | 1.00 | 0.77 | 1SG | 705 |
| ATOM | 705 | N | PHE | 87 | 6.202 | 29.668 | 7.637 | 1.00 | 0.79 | 1SG | 706 |
| ATOM | 706 | CA | PHE | 87 | 5.354 | 28.707 | 8.263 | 1.00 | 0.79 | 1SG | 707 |
| ATOM | 707 | CB | PHE | 87 | 4.409 | 29.317 | 9.320 | 1.00 | 0.79 | 1SG | 708 |
| ATOM | 708 | CG | PHE | 87 | 5.205 | 29.612 | 10.558 | 1.00 | 0.79 | 1SG | 709 |
| ATOM | 709 | CD1 | PHE | 87 | 6.077 | 30.674 | 10.626 | 1.00 | 0.79 | 1SG | 710 |
| ATOM | 710 | CD2 | PHE | 87 | 5.072 | 28.829 | 11.681 | 1.00 | 0.79 | 1SG | 711 |
| ATOM | 711 | CE1 | PHE | 87 | 6.805 | 30.950 | 11.759 | 1.00 | 0.79 | 1SG | 712 |
| ATOM | 712 | CE2 | PHE | 87 | 5.794 | 29.093 | 12.823 | 1.00 | 0.79 | 1SG | 713 |
| ATOM | 713 | CZ | PHE | 87 | 6.665 | 30.154 | 12.868 | 1.00 | 0.79 | 1SG | 714 |
| ATOM | 714 | C | PHE | 87 | 4.559 | 27.900 | 7.267 | 1.00 | 0.79 | 1SG | 715 |
| ATOM | 715 | O | PHE | 87 | 3.348 | 28.073 | 7.130 | 1.00 | 0.79 | 1SG | 716 |
| ATOM | 716 | N | PRO | 88 | 5.240 | 26.965 | 6.597 | 1.00 | 0.99 | 1SG | 717 |
| ATOM | 717 | CA | PRO | 88 | 4.611 | 25.895 | 5.827 | 1.00 | 0.99 | 1SG | 718 |
| ATOM | 718 | CD | PRO | 88 | 6.541 | 27.300 | 6.029 | 1.00 | 0.99 | 1SG | 719 |
| ATOM | 719 | CB | PRO | 88 | 5.634 | 25.440 | 4.787 | 1.00 | 0.99 | 1SG | 720 |
| ATOM | 720 | CG | PRO | 88 | 6.593 | 26.628 | 4.650 | 1.00 | 0.99 | 1SG | 721 |
| ATOM | 721 | C | PRO | 88 | 4.380 | 24.812 | 6.851 | 1.00 | 0.99 | 1SG | 722 |
| ATOM | 722 | O | PRO | 88 | 3.878 | 23.730 | 6.557 | 1.00 | 0.99 | 1SG | 723 |
| ATOM | 723 | N | ILE | 89 | 4.610 | 25.283 | 8.071 | 1.00 | 0.98 | 1SG | 724 |
| ATOM | 724 | CA | ILE | 89 | 4.748 | 25.109 | 9.465 | 1.00 | 0.98 | 1SG | 725 |
| ATOM | 725 | CB | ILE | 89 | 5.397 | 26.235 | 10.206 | 1.00 | 0.98 | 1SG | 726 |
| ATOM | 726 | CG2 | ILE | 89 | 5.277 | 25.891 | 11.699 | 1.00 | 0.98 | 1SG | 727 |
| ATOM | 727 | CG1 | ILE | 89 | 6.852 | 26.431 | 9.748 | 1.00 | 0.98 | 1SG | 728 |
| ATOM | 728 | CD1 | ILE | 89 | 7.749 | 25.227 | 10.039 | 1.00 | 0.98 | 1SG | 729 |
| ATOM | 729 | C | ILE | 89 | 3.346 | 25.054 | 9.874 | 1.00 | 0.98 | 1SG | 730 |
| ATOM | 730 | O | ILE | 89 | 2.731 | 26.066 | 10.188 | 1.00 | 0.98 | 1SG | 731 |
| ATOM | 731 | N | ALA | 90 | 2.816 | 23.828 | 9.840 | 1.00 | 0.69 | 1SG | 732 |
| ATOM | 732 | CA | ALA | 90 | 1.583 | 23.150 | 9.631 | 1.00 | 0.69 | 1SG | 733 |
| ATOM | 733 | CB | ALA | 90 | 1.328 | 22.124 | 10.746 | 1.00 | 0.69 | 1SG | 734 |
| ATOM | 734 | C | ALA | 90 | 0.338 | 23.956 | 9.521 | 1.00 | 0.69 | 1SG | 735 |
| ATOM | 735 | O | ALA | 90 | −0.721 | 23.409 | 9.815 | 1.00 | 0.69 | 1SG | 736 |
| ATOM | 736 | N | TYR | 91 | 0.428 | 25.281 | 9.300 | 1.00 | 0.46 | 1SG | 737 |
| ATOM | 737 | CA | TYR | 91 | −0.776 | 25.924 | 8.840 | 1.00 | 0.46 | 1SG | 738 |
| ATOM | 738 | CB | TYR | 91 | −0.541 | 27.264 | 8.103 | 1.00 | 0.46 | 1SG | 739 |
| ATOM | 739 | CG | TYR | 91 | −0.308 | 28.396 | 9.036 | 1.00 | 0.46 | 1SG | 740 |
| ATOM | 740 | CD1 | TYR | 91 | 0.921 | 28.629 | 9.611 | 1.00 | 0.46 | 1SG | 741 |
| ATOM | 741 | CD2 | TYR | 91 | −1.348 | 29.258 | 9.304 | 1.00 | 0.46 | 1SG | 742 |
| ATOM | 742 | CE1 | TYR | 91 | 1.090 | 29.687 | 10.476 | 1.00 | 0.46 | 1SG | 743 |
| ATOM | 743 | CE2 | TYR | 91 | −1.184 | 30.317 | 10.164 | 1.00 | 0.46 | 1SG | 744 |
| ATOM | 744 | CZ | TYR | 91 | 0.038 | 30.531 | 10.749 | 1.00 | 0.46 | 1SG | 745 |
| ATOM | 745 | OH | TYR | 91 | 0.209 | 31.620 | 11.631 | 1.00 | 0.46 | 1SG | 746 |
| ATOM | 746 | C | TYR | 91 | −1.305 | 25.035 | 7.744 | 1.00 | 0.46 | 1SG | 747 |
| ATOM | 747 | O | TYR | 91 | −2.496 | 24.727 | 7.704 | 1.00 | 0.46 | 1SG | 748 |
| ATOM | 748 | N | SER | 92 | −0.414 | 24.565 | 6.848 | 1.00 | 0.44 | 1SG | 749 |
| ATOM | 749 | CA | SER | 92 | −0.820 | 23.684 | 5.785 | 1.00 | 0.44 | 1SG | 750 |
| ATOM | 750 | CB | SER | 92 | 0.305 | 23.399 | 4.774 | 1.00 | 0.44 | 1SG | 751 |
| ATOM | 751 | OG | SER | 92 | 1.343 | 22.649 | 5.387 | 1.00 | 0.44 | 1SG | 752 |
| ATOM | 752 | C | SER | 92 | −1.259 | 22.367 | 6.355 | 1.00 | 0.44 | 1SG | 753 |
| ATOM | 753 | O | SER | 92 | −2.234 | 21.775 | 5.895 | 1.00 | 0.44 | 1SG | 754 |
| ATOM | 754 | N | LEU | 93 | −0.547 | 21.859 | 7.379 | 1.00 | 0.37 | 1SG | 755 |
| ATOM | 755 | CA | LEU | 93 | −0.878 | 20.565 | 7.915 | 1.00 | 0.37 | 1SG | 756 |
| ATOM | 756 | CB | LEU | 93 | 0.112 | 20.091 | 8.995 | 1.00 | 0.37 | 1SG | 757 |
| ATOM | 757 | CG | LEU | 93 | 1.518 | 19.757 | 8.454 | 1.00 | 0.37 | 1SG | 758 |
| ATOM | 758 | CD2 | LEU | 93 | 2.414 | 19.188 | 9.566 | 1.00 | 0.37 | 1SG | 759 |
| ATOM | 759 | CD1 | LEU | 93 | 2.160 | 20.960 | 7.745 | 1.00 | 0.37 | 1SG | 760 |
| ATOM | 760 | C | LEU | 93 | −2.248 | 20.587 | 8.521 | 1.00 | 0.37 | 1SG | 761 |
| ATOM | 761 | O | LEU | 93 | −3.041 | 19.667 | 8.322 | 1.00 | 0.37 | 1SG | 762 |
| ATOM | 762 | N | VAL | 94 | −2.567 | 21.656 | 9.267 | 1.00 | 0.28 | 1SG | 763 |
| ATOM | 763 | CA | VAL | 94 | −3.814 | 21.761 | 9.965 | 1.00 | 0.28 | 1SG | 764 |
| ATOM | 764 | CB | VAL | 94 | −3.918 | 23.043 | 10.738 | 1.00 | 0.28 | 1SG | 765 |
| ATOM | 765 | CG1 | VAL | 94 | −5.311 | 23.125 | 11.384 | 1.00 | 0.28 | 1SG | 766 |
| ATOM | 766 | CG2 | VAL | 94 | −2.754 | 23.105 | 11.742 | 1.00 | 0.28 | 1SG | 767 |
| ATOM | 767 | C | VAL | 94 | −4.936 | 21.745 | 8.974 | 1.00 | 0.28 | 1SG | 768 |
| ATOM | 768 | O | VAL | 94 | −5.935 | 21.054 | 9.169 | 1.00 | 0.28 | 1SG | 769 |
| ATOM | 769 | N | VAL | 95 | −4.795 | 22.496 | 7.866 | 1.00 | 0.37 | 1SG | 770 |
| ATOM | 770 | CA | VAL | 95 | −5.878 | 22.574 | 6.928 | 1.00 | 0.37 | 1SG | 771 |
| ATOM | 771 | CB | VAL | 95 | −5.618 | 23.545 | 5.804 | 1.00 | 0.37 | 1SG | 772 |
| ATOM | 772 | CG1 | VAL | 95 | −4.459 | 23.040 | 4.931 | 1.00 | 0.37 | 1SG | 773 |
| ATOM | 773 | CG2 | VAL | 95 | −6.930 | 23.759 | 5.031 | 1.00 | 0.37 | 1SG | 774 |
| ATOM | 774 | C | VAL | 95 | −6.143 | 21.223 | 6.348 | 1.00 | 0.37 | 1SG | 775 |
| ATOM | 775 | O | VAL | 95 | −7.292 | 20.801 | 6.221 | 1.00 | 0.37 | 1SG | 776 |
| ATOM | 776 | N | HIS | 96 | −5.079 | 20.494 | 5.981 | 1.00 | 0.64 | 1SG | 777 |
| ATOM | 777 | CA | HIS | 96 | −5.288 | 19.210 | 5.385 | 1.00 | 0.64 | 1SG | 778 |
| ATOM | 778 | ND1 | HIS | 96 | −2.715 | 20.280 | 3.369 | 1.00 | 0.64 | 1SG | 779 |
| ATOM | 779 | CG | HIS | 96 | −3.632 | 19.269 | 3.536 | 1.00 | 0.64 | 1SG | 780 |
| ATOM | 780 | CB | HIS | 96 | −3.996 | 18.619 | 4.828 | 1.00 | 0.64 | 1SG | 781 |
| ATOM | 781 | NE2 | HIS | 96 | −3.504 | 19.790 | 1.348 | 1.00 | 0.64 | 1SG | 782 |
| ATOM | 782 | CD2 | HIS | 96 | −4.100 | 18.989 | 2.296 | 1.00 | 0.64 | 1SG | 783 |
| ATOM | 783 | CE1 | HIS | 96 | −2.677 | 20.554 | 2.041 | 1.00 | 0.64 | 1SG | 784 |
| ATOM | 784 | C | HIS | 96 | −5.869 | 18.295 | 6.404 | 1.00 | 0.64 | 1SG | 785 |
| ATOM | 785 | O | HIS | 96 | −6.730 | 17.466 | 6.109 | 1.00 | 0.64 | 1SG | 786 |
| ATOM | 786 | N | LYS | 97 | −5.397 | 18.431 | 7.648 | 1.00 | 0.82 | 1SG | 787 |
| ATOM | 787 | CA | LYS | 97 | −5.905 | 17.634 | 8.717 | 1.00 | 0.82 | 1SG | 788 |
| ATOM | 788 | CB | LYS | 97 | −5.146 | 17.935 | 10.024 | 1.00 | 0.82 | 1SG | 789 |
| ATOM | 789 | CG | LYS | 97 | −5.648 | 17.188 | 11.256 | 1.00 | 0.82 | 1SG | 790 |
| ATOM | 790 | CD | LYS | 97 | −6.985 | 17.711 | 11.774 | 1.00 | 0.82 | 1SG | 791 |
| ATOM | 791 | CE | LYS | 97 | −6.844 | 18.813 | 12.820 | 1.00 | 0.82 | 1SG | 792 |
| ATOM | 792 | NZ | LYS | 97 | −6.281 | 18.254 | 14.070 | 1.00 | 0.82 | 1SG | 793 |
| ATOM | 793 | C | LYS | 97 | −7.362 | 17.954 | 8.868 | 1.00 | 0.82 | 1SG | 794 |
| ATOM | 794 | O | LYS | 97 | −8.165 | 17.083 | 9.196 | 1.00 | 0.82 | 1SG | 795 |
| ATOM | 795 | N | ASP | 98 | −7.754 | 19.227 | 8.684 | 1.00 | 0.63 | 1SG | 796 |
| ATOM | 796 | CA | ASP | 98 | −9.152 | 19.527 | 8.810 | 1.00 | 0.63 | 1SG | 797 |
| ATOM | 797 | CB | ASP | 98 | −9.436 | 21.043 | 8.806 | 1.00 | 0.63 | 1SG | 798 |
| ATOM | 798 | CG | ASP | 98 | −10.908 | 21.293 | 9.133 | 1.00 | 0.63 | 1SG | 799 |
| ATOM | 799 | OD1 | ASP | 98 | −11.789 | 20.803 | 8.376 | 1.00 | 0.63 | 1SG | 800 |
| ATOM | 800 | OD2 | ASP | 98 | −11.167 | 21.999 | 10.144 | 1.00 | 0.63 | 1SG | 801 |
| ATOM | 801 | C | ASP | 98 | −9.918 | 18.931 | 7.669 | 1.00 | 0.63 | 1SG | 802 |
| ATOM | 802 | O | ASP | 98 | −10.819 | 18.122 | 7.876 | 1.00 | 0.63 | 1SG | 803 |
| ATOM | 803 | N | ALA | 99 | −9.535 | 19.281 | 6.424 | 1.00 | 0.55 | 1SG | 804 |
| ATOM | 804 | CA | ALA | 99 | −10.300 | 18.920 | 5.260 | 1.00 | 0.55 | 1SG | 805 |
| ATOM | 805 | CB | ALA | 99 | −9.738 | 19.533 | 3.967 | 1.00 | 0.55 | 1SG | 806 |
| ATOM | 806 | C | ALA | 99 | −10.316 | 17.452 | 5.051 | 1.00 | 0.55 | 1SG | 807 |
| ATOM | 807 | O | ALA | 99 | −11.391 | 16.876 | 4.877 | 1.00 | 0.55 | 1SG | 808 |
| ATOM | 808 | N | ILE | 100 | −9.105 | 16.860 | 5.116 | 1.00 | 0.80 | 1SG | 809 |
| ATOM | 809 | CA | ILE | 100 | −8.760 | 15.501 | 4.818 | 1.00 | 0.80 | 1SG | 810 |
| ATOM | 810 | CB | ILE | 100 | −7.767 | 14.928 | 5.757 | 1.00 | 0.80 | 1SG | 811 |
| ATOM | 811 | CG2 | ILE | 100 | −8.270 | 15.309 | 7.139 | 1.00 | 0.80 | 1SG | 812 |
| ATOM | 812 | CG1 | ILE | 100 | −7.520 | 13.436 | 5.459 | 1.00 | 0.80 | 1SG | 813 |
| ATOM | 813 | CD1 | ILE | 100 | −6.218 | 12.879 | 6.033 | 1.00 | 0.80 | 1SG | 814 |
| ATOM | 814 | C | ILE | 100 | −9.932 | 14.624 | 4.639 | 1.00 | 0.80 | 1SG | 815 |
| ATOM | 815 | O | ILE | 100 | −10.325 | 13.840 | 5.509 | 1.00 | 0.80 | 1SG | 816 |
| ATOM | 816 | N | MET | 101 | −10.527 | 14.798 | 3.437 | 1.00 | 0.82 | 1SG | 817 |
| ATOM | 817 | CA | MET | 101 | −11.591 | 13.970 | 2.990 | 1.00 | 0.82 | 1SG | 818 |
| ATOM | 818 | CB | MET | 101 | −12.531 | 14.602 | 1.943 | 1.00 | 0.82 | 1SG | 819 |
| ATOM | 819 | CG | MET | 101 | −11.949 | 14.716 | 0.530 | 1.00 | 0.82 | 1SG | 820 |
| ATOM | 820 | SD | MET | 101 | −13.203 | 14.991 | −0.760 | 1.00 | 0.82 | 1SG | 821 |
| ATOM | 821 | CE | MET | 101 | −13.775 | 16.577 | −0.082 | 1.00 | 0.82 | 1SG | 822 |
| ATOM | 822 | C | MET | 101 | −10.909 | 12.827 | 2.333 | 1.00 | 0.82 | 1SG | 823 |
| ATOM | 823 | O | MET | 101 | −11.473 | 12.151 | 1.474 | 1.00 | 0.82 | 1SG | 824 |
| ATOM | 824 | N | VAL | 102 | −9.659 | 12.579 | 2.753 | 1.00 | 0.67 | 1SG | 825 |
| ATOM | 825 | CA | VAL | 102 | −9.019 | 11.397 | 2.325 | 1.00 | 0.67 | 1SG | 826 |
| ATOM | 826 | CB | VAL | 102 | −7.648 | 11.626 | 1.830 | 1.00 | 0.67 | 1SG | 827 |
| ATOM | 827 | CG1 | VAL | 102 | −7.090 | 10.269 | 1.389 | 1.00 | 0.67 | 1SG | 828 |
| ATOM | 828 | CG2 | VAL | 102 | −7.718 | 12.727 | 0.762 | 1.00 | 0.67 | 1SG | 829 |
| ATOM | 829 | C | VAL | 102 | −8.915 | 10.619 | 3.590 | 1.00 | 0.67 | 1SG | 830 |
| ATOM | 830 | O | VAL | 102 | −7.832 | 10.261 | 4.055 | 1.00 | 0.67 | 1SG | 831 |
| ATOM | 831 | N | GLU | 103 | −10.107 | 10.363 | 4.155 | 1.00 | 0.78 | 1SG | 832 |
| ATOM | 832 | CA | GLU | 103 | −10.419 | 9.620 | 5.340 | 1.00 | 0.78 | 1SG | 833 |
| ATOM | 833 | CB | GLU | 103 | −10.086 | 8.129 | 5.237 | 1.00 | 0.78 | 1SG | 834 |
| ATOM | 834 | CG | GLU | 103 | −10.957 | 7.401 | 4.219 | 1.00 | 0.78 | 1SG | 835 |
| ATOM | 835 | CD | GLU | 103 | −10.857 | 5.914 | 4.522 | 1.00 | 0.78 | 1SG | 836 |
| ATOM | 836 | OE1 | GLU | 103 | −11.218 | 5.522 | 5.664 | 1.00 | 0.78 | 1SG | 837 |
| ATOM | 837 | OE2 | GLU | 103 | −10.429 | 5.149 | 3.618 | 1.00 | 0.78 | 1SG | 838 |
| ATOM | 838 | C | GLU | 103 | −9.822 | 10.118 | 6.623 | 1.00 | 0.78 | 1SG | 839 |
| ATOM | 839 | O | GLU | 103 | −9.644 | 9.308 | 7.529 | 1.00 | 0.78 | 1SG | 840 |
| ATOM | 840 | N | ARG | 104 | −9.513 | 11.419 | 6.802 | 1.00 | 0.85 | 1SG | 841 |
| ATOM | 841 | CA | ARG | 104 | −9.134 | 11.740 | 8.154 | 1.00 | 0.85 | 1SG | 842 |
| ATOM | 842 | CB | ARG | 104 | −7.955 | 12.698 | 8.381 | 1.00 | 0.85 | 1SG | 843 |
| ATOM | 843 | CG | ARG | 104 | −7.408 | 12.765 | 9.814 | 1.00 | 0.85 | 1SG | 844 |
| ATOM | 844 | CD | ARG | 104 | −7.957 | 13.885 | 10.702 | 1.00 | 0.85 | 1SG | 845 |
| ATOM | 845 | NE | ARG | 104 | −7.272 | 13.769 | 12.033 | 1.00 | 0.85 | 1SG | 846 |
| ATOM | 846 | CZ | ARG | 104 | −7.562 | 14.612 | 13.075 | 1.00 | 0.85 | 1SG | 847 |
| ATOM | 847 | NH1 | ARG | 104 | −8.491 | 15.598 | 12.949 | 1.00 | 0.85 | 1SG | 848 |
| ATOM | 848 | NH2 | ARG | 104 | −6.933 | 14.487 | 14.279 | 1.00 | 0.85 | 1SG | 849 |
| ATOM | 849 | C | ARG | 104 | −10.345 | 12.353 | 8.745 | 1.00 | 0.85 | 1SG | 850 |
| ATOM | 850 | O | ARG | 104 | −11.356 | 11.667 | 8.876 | 1.00 | 0.85 | 1SG | 851 |
| ATOM | 851 | N | LEU | 105 | −10.311 | 13.646 | 9.131 | 1.00 | 0.64 | 1SG | 852 |
| ATOM | 852 | CA | LEU | 105 | −11.537 | 14.070 | 9.739 | 1.00 | 0.64 | 1SG | 853 |
| ATOM | 853 | CB | LEU | 105 | −11.548 | 15.439 | 10.431 | 1.00 | 0.64 | 1SG | 854 |
| ATOM | 854 | CG | LEU | 105 | −11.098 | 15.330 | 11.886 | 1.00 | 0.64 | 1SG | 855 |
| ATOM | 855 | CD2 | LEU | 105 | −11.848 | 14.212 | 12.627 | 1.00 | 0.64 | 1SG | 856 |
| ATOM | 856 | CD1 | LEU | 105 | −11.172 | 16.684 | 12.599 | 1.00 | 0.64 | 1SG | 857 |
| ATOM | 857 | C | LEU | 105 | −12.701 | 14.064 | 8.813 | 1.00 | 0.64 | 1SG | 858 |
| ATOM | 858 | O | LEU | 105 | −13.636 | 13.293 | 9.015 | 1.00 | 0.64 | 1SG | 859 |
| ATOM | 859 | N | ILE | 106 | −12.685 | 14.893 | 7.757 | 1.00 | 0.51 | 1SG | 860 |
| ATOM | 860 | CA | ILE | 106 | −13.891 | 14.953 | 6.986 | 1.00 | 0.51 | 1SG | 861 |
| ATOM | 861 | CB | ILE | 106 | −13.909 | 16.026 | 5.936 | 1.00 | 0.51 | 1SG | 862 |
| ATOM | 862 | CG2 | ILE | 106 | −14.770 | 15.530 | 4.762 | 1.00 | 0.51 | 1SG | 863 |
| ATOM | 863 | CG1 | ILE | 106 | −14.392 | 17.352 | 6.553 | 1.00 | 0.51 | 1SG | 864 |
| ATOM | 864 | CD1 | ILE | 106 | −13.534 | 17.878 | 7.696 | 1.00 | 0.51 | 1SG | 865 |
| ATOM | 865 | C | ILE | 106 | −14.246 | 13.664 | 6.327 | 1.00 | 0.51 | 1SG | 866 |
| ATOM | 866 | O | ILE | 106 | −15.365 | 13.185 | 6.505 | 1.00 | 0.51 | 1SG | 867 |
| ATOM | 867 | N | HIS | 107 | −13.326 | 13.047 | 5.567 | 1.00 | 0.42 | 1SG | 868 |
| ATOM | 868 | CA | HIS | 107 | −13.807 | 11.895 | 4.869 | 1.00 | 0.42 | 1SG | 869 |
| ATOM | 869 | ND1 | HIS | 107 | −14.241 | 10.581 | 1.909 | 1.00 | 0.42 | 1SG | 870 |
| ATOM | 870 | CG | HIS | 107 | −13.340 | 10.282 | 2.910 | 1.00 | 0.42 | 1SG | 871 |
| ATOM | 871 | CB | HIS | 107 | −12.801 | 11.277 | 3.899 | 1.00 | 0.42 | 1SG | 872 |
| ATOM | 872 | NE2 | HIS | 107 | −13.744 | 8.425 | 1.686 | 1.00 | 0.42 | 1SG | 873 |
| ATOM | 873 | CD2 | HIS | 107 | −13.047 | 8.960 | 2.755 | 1.00 | 0.42 | 1SG | 874 |
| ATOM | 874 | CE1 | HIS | 107 | −14.447 | 9.437 | 1.208 | 1.00 | 0.42 | 1SG | 875 |
| ATOM | 875 | C | HIS | 107 | −14.136 | 10.826 | 5.843 | 1.00 | 0.42 | 1SG | 876 |
| ATOM | 876 | O | HIS | 107 | −15.243 | 10.287 | 5.821 | 1.00 | 0.42 | 1SG | 877 |
| ATOM | 877 | N | ALA | 108 | −13.198 | 10.529 | 6.761 | 1.00 | 0.37 | 1SG | 878 |
| ATOM | 878 | CA | ALA | 108 | −13.413 | 9.402 | 7.614 | 1.00 | 0.37 | 1SG | 879 |
| ATOM | 879 | CB | ALA | 108 | −12.226 | 9.078 | 8.535 | 1.00 | 0.37 | 1SG | 880 |
| ATOM | 880 | C | ALA | 108 | −14.596 | 9.626 | 8.492 | 1.00 | 0.37 | 1SG | 881 |
| ATOM | 881 | O | ALA | 108 | −15.509 | 8.805 | 8.532 | 1.00 | 0.37 | 1SG | 882 |
| ATOM | 882 | N | ILE | 109 | −14.607 | 10.750 | 9.232 | 1.00 | 0.74 | 1SG | 883 |
| ATOM | 883 | CA | ILE | 109 | −15.668 | 10.969 | 10.173 | 1.00 | 0.74 | 1SG | 884 |
| ATOM | 884 | CB | ILE | 109 | −15.332 | 11.977 | 11.226 | 1.00 | 0.74 | 1SG | 885 |
| ATOM | 885 | CG2 | ILE | 109 | −16.580 | 12.177 | 12.099 | 1.00 | 0.74 | 1SG | 886 |
| ATOM | 886 | CG1 | ILE | 109 | −14.094 | 11.523 | 12.026 | 1.00 | 0.74 | 1SG | 887 |
| ATOM | 887 | CD1 | ILE | 109 | −14.277 | 10.201 | 12.767 | 1.00 | 0.74 | 1SG | 888 |
| ATOM | 888 | C | ILE | 109 | −16.963 | 11.371 | 9.530 | 1.00 | 0.74 | 1SG | 889 |
| ATOM | 889 | O | ILE | 109 | −18.023 | 10.895 | 9.933 | 1.00 | 0.74 | 1SG | 890 |
| ATOM | 890 | N | TYR | 110 | −16.958 | 12.255 | 8.516 | 1.00 | 1.09 | 1SG | 891 |
| ATOM | 891 | CA | TYR | 110 | −18.269 | 12.618 | 8.090 | 1.00 | 1.09 | 1SG | 892 |
| ATOM | 892 | CB | TYR | 110 | −18.641 | 14.069 | 8.435 | 1.00 | 1.09 | 1SG | 893 |
| ATOM | 893 | CG | TYR | 110 | −20.114 | 14.030 | 8.632 | 1.00 | 1.09 | 1SG | 894 |
| ATOM | 894 | CD1 | TYR | 110 | −20.596 | 13.581 | 9.844 | 1.00 | 1.09 | 1SG | 895 |
| ATOM | 895 | CD2 | TYR | 110 | −21.022 | 14.400 | 7.660 | 1.00 | 1.09 | 1SG | 896 |
| ATOM | 896 | CE1 | TYR | 110 | −21.940 | 13.507 | 10.108 | 1.00 | 1.09 | 1SG | 897 |
| ATOM | 897 | CE2 | TYR | 110 | −22.376 | 14.332 | 7.916 | 1.00 | 1.09 | 1SG | 898 |
| ATOM | 898 | CZ | TYR | 110 | −22.833 | 13.885 | 9.134 | 1.00 | 1.09 | 1SG | 899 |
| ATOM | 899 | OH | TYR | 110 | −24.225 | 13.818 | 9.377 | 1.00 | 1.09 | 1SG | 900 |
| ATOM | 900 | C | TYR | 110 | −18.314 | 12.439 | 6.612 | 1.00 | 1.09 | 1SG | 901 |
| ATOM | 901 | O | TYR | 110 | −18.111 | 13.387 | 5.855 | 1.00 | 1.09 | 1SG | 902 |
| ATOM | 902 | N | ASN | 111 | −18.568 | 11.177 | 6.188 | 1.00 | 1.03 | 1SG | 903 |
| ATOM | 903 | CA | ASN | 111 | −18.704 | 10.686 | 4.834 | 1.00 | 1.03 | 1SG | 904 |
| ATOM | 904 | CB | ASN | 111 | −17.641 | 11.111 | 3.827 | 1.00 | 1.03 | 1SG | 905 |
| ATOM | 905 | CG | ASN | 111 | −17.997 | 12.471 | 3.256 | 1.00 | 1.03 | 1SG | 906 |
| ATOM | 906 | OD1 | ASN | 111 | −17.047 | 11.685 | 3.301 | 1.00 | 1.03 | 1SG | 907 |
| ATOM | 907 | ND2 | ASN | 111 | −19.305 | 12.496 | 3.650 | 1.00 | 1.03 | 1SG | 908 |
| ATOM | 908 | C | ASN | 111 | −18.611 | 9.204 | 4.967 | 1.00 | 1.03 | 1SG | 909 |
| ATOM | 909 | O | ASN | 111 | −19.629 | 8.523 | 4.953 | 1.00 | 1.03 | 1SG | 910 |
| ATOM | 910 | N | GLN | 112 | −17.387 | 8.669 | 5.143 | 1.00 | 0.85 | 1SG | 911 |
| ATOM | 911 | CA | GLN | 112 | −17.219 | 7.269 | 5.393 | 1.00 | 0.85 | 1SG | 912 |
| ATOM | 912 | CB | GLN | 112 | −15.761 | 6.897 | 5.685 | 1.00 | 0.85 | 1SG | 913 |
| ATOM | 913 | CG | GLN | 112 | −14.904 | 6.981 | 4.436 | 1.00 | 0.85 | 1SG | 914 |
| ATOM | 914 | CD | GLN | 112 | −15.473 | 5.931 | 3.502 | 1.00 | 0.85 | 1SG | 915 |
| ATOM | 915 | OE1 | GLN | 112 | −15.987 | 6.279 | 2.442 | 1.00 | 0.85 | 1SG | 916 |
| ATOM | 916 | NE2 | GLN | 112 | −15.393 | 4.631 | 3.900 | 1.00 | 0.85 | 1SG | 917 |
| ATOM | 917 | C | GLN | 112 | −17.968 | 7.054 | 6.657 | 1.00 | 0.85 | 1SG | 918 |
| ATOM | 918 | O | GLN | 112 | −18.612 | 6.032 | 6.891 | 1.00 | 0.85 | 1SG | 919 |
| ATOM | 919 | N | HIS | 113 | −17.900 | 8.095 | 7.490 | 1.00 | 0.63 | 1SG | 920 |
| ATOM | 920 | CA | HIS | 113 | −18.571 | 8.164 | 8.745 | 1.00 | 0.63 | 1SG | 921 |
| ATOM | 921 | ND1 | HIS | 113 | −21.170 | 7.160 | 6.629 | 1.00 | 0.63 | 1SG | 922 |
| ATOM | 922 | CG | HIS | 113 | −20.935 | 7.363 | 7.973 | 1.00 | 0.63 | 1SG | 923 |
| ATOM | 923 | CB | HIS | 113 | −20.090 | 8.451 | 8.581 | 1.00 | 0.63 | 1SG | 924 |
| ATOM | 924 | NE2 | HIS | 113 | −22.302 | 5.585 | 7.714 | 1.00 | 0.63 | 1SG | 925 |
| ATOM | 925 | CD2 | HIS | 113 | −21.637 | 6.394 | 8.617 | 1.00 | 0.63 | 1SG | 926 |
| ATOM | 926 | CE1 | HIS | 113 | −21.995 | 6.085 | 6.533 | 1.00 | 0.63 | 1SG | 927 |
| ATOM | 927 | C | HIS | 113 | −18.278 | 6.967 | 9.605 | 1.00 | 0.63 | 1SG | 928 |
| ATOM | 928 | O | HIS | 113 | −19.137 | 6.112 | 9.818 | 1.00 | 0.63 | 1SG | 929 |
| ATOM | 929 | N | ASN | 114 | −17.022 | 6.845 | 10.099 | 1.00 | 0.46 | 1SG | 930 |
| ATOM | 930 | CA | ASN | 114 | −16.754 | 5.754 | 10.988 | 1.00 | 0.46 | 1SG | 931 |
| ATOM | 931 | CB | ASN | 114 | −15.546 | 4.897 | 10.603 | 1.00 | 0.46 | 1SG | 932 |
| ATOM | 932 | CG | ASN | 114 | −16.055 | 3.964 | 9.509 | 1.00 | 0.46 | 1SG | 933 |
| ATOM | 933 | OD1 | ASN | 114 | −15.606 | 3.971 | 8.365 | 1.00 | 0.46 | 1SG | 934 |
| ATOM | 934 | ND2 | ASN | 114 | −17.049 | 3.117 | 9.884 | 1.00 | 0.46 | 1SG | 935 |
| ATOM | 935 | C | ASN | 114 | −16.602 | 6.289 | 12.373 | 1.00 | 0.46 | 1SG | 936 |
| ATOM | 936 | O | ASN | 114 | −16.357 | 7.477 | 12.565 | 1.00 | 0.46 | 1SG | 937 |
| ATOM | 937 | N | ILE | 115 | −16.779 | 5.402 | 13.373 | 1.00 | 0.42 | 1SG | 938 |
| ATOM | 938 | CA | ILE | 115 | −16.796 | 5.737 | 14.774 | 1.00 | 0.42 | 1SG | 939 |
| ATOM | 939 | CB | ILE | 115 | −17.295 | 4.620 | 15.643 | 1.00 | 0.42 | 1SG | 940 |
| ATOM | 940 | CG2 | ILE | 115 | −18.765 | 4.356 | 15.281 | 1.00 | 0.42 | 1SG | 941 |
| ATOM | 941 | CG1 | ILE | 115 | −16.395 | 3.376 | 15.542 | 1.00 | 0.42 | 1SG | 942 |
| ATOM | 942 | CD1 | ILE | 115 | −16.745 | 2.300 | 16.572 | 1.00 | 0.42 | 1SG | 943 |
| ATOM | 943 | C | ILE | 115 | −15.466 | 6.179 | 15.308 | 1.00 | 0.42 | 1SG | 944 |
| ATOM | 944 | O | ILE | 115 | −15.416 | 7.036 | 16.187 | 1.00 | 0.42 | 1SG | 945 |
| ATOM | 945 | N | TYR | 116 | −14.343 | 5.598 | 14.851 | 1.00 | 0.29 | 1SG | 946 |
| ATOM | 946 | CA | TYR | 116 | −13.101 | 6.027 | 15.442 | 1.00 | 0.29 | 1SG | 947 |
| ATOM | 947 | CB | TYR | 116 | −12.551 | 5.071 | 16.534 | 1.00 | 0.29 | 1SG | 948 |
| ATOM | 948 | CG | TYR | 116 | −13.373 | 5.117 | 17.780 | 1.00 | 0.29 | 1SG | 949 |
| ATOM | 949 | CD1 | TYR | 116 | −14.565 | 4.433 | 17.871 | 1.00 | 0.29 | 1SG | 950 |
| ATOM | 950 | CD2 | TYR | 116 | −12.945 | 5.835 | 18.882 | 1.00 | 0.29 | 1SG | 951 |
| ATOM | 951 | CE1 | TYR | 116 | −15.315 | 4.461 | 19.025 | 1.00 | 0.29 | 1SG | 952 |
| ATOM | 952 | CE2 | TYR | 116 | −13.681 | 5.872 | 20.043 | 1.00 | 0.29 | 1SG | 953 |
| ATOM | 953 | CZ | TYR | 116 | −14.872 | 5.184 | 20.107 | 1.00 | 0.29 | 1SG | 954 |
| ATOM | 954 | OH | TYR | 116 | −15.646 | 5.204 | 21.288 | 1.00 | 0.29 | 1SG | 955 |
| ATOM | 955 | C | TYR | 116 | −12.057 | 6.071 | 14.367 | 1.00 | 0.29 | 1SG | 956 |
| ATOM | 956 | O | TYR | 116 | −12.300 | 5.630 | 13.245 | 1.00 | 0.29 | 1SG | 957 |
| ATOM | 957 | N | CYS | 117 | −10.870 | 6.640 | 14.689 | 1.00 | 0.18 | 1SG | 958 |
| ATOM | 958 | CA | CYS | 117 | −9.802 | 6.705 | 13.726 | 1.00 | 0.18 | 1SG | 959 |
| ATOM | 959 | CB | CYS | 117 | −9.883 | 7.9391 | 2.804 | 1.00 | 0.18 | 1SG | 960 |
| ATOM | 960 | SG | CYS | 117 | −11.387 | 7.977 | 11.774 | 1.00 | 0.18 | 1SG | 961 |
| ATOM | 961 | C | CYS | 117 | −8.479 | 6.777 | 14.445 | 1.00 | 0.18 | 1SG | 962 |
| ATOM | 962 | O | CYS | 117 | −8.374 | 7.292 | 15.558 | 1.00 | 0.18 | 1SG | 963 |
| ATOM | 963 | N | ILE | 118 | −7.422 | 6.237 | 13.806 | 1.00 | 0.28 | 1SG | 964 |
| ATOM | 964 | CA | ILE | 118 | −6.077 | 6.329 | 14.318 | 1.00 | 0.28 | 1SG | 965 |
| ATOM | 965 | CB | ILE | 118 | −5.393 | 4.987 | 14.296 | 1.00 | 0.28 | 1SG | 966 |
| ATOM | 966 | CG2 | ILE | 118 | −3.977 | 5.142 | 14.861 | 1.00 | 0.28 | 1SG | 967 |
| ATOM | 967 | CG1 | ILE | 118 | −6.210 | 3.964 | 15.085 | 1.00 | 0.28 | 1SG | 968 |
| ATOM | 968 | CD1 | ILE | 118 | −5.782 | 2.520 | 14.827 | 1.00 | 0.28 | 1SG | 969 |
| ATOM | 969 | C | ILE | 118 | −5.395 | 7.165 | 13.290 | 1.00 | 0.28 | 1SG | 970 |
| ATOM | 970 | O | ILE | 118 | −5.282 | 6.750 | 12.138 | 1.00 | 0.28 | 1SG | 971 |
| ATOM | 971 | N | HIS | 119 | −4.887 | 8.358 | 13.653 | 1.00 | 0.50 | 1SG | 972 |
| ATOM | 972 | CA | HIS | 119 | −4.541 | 9.200 | 12.545 | 1.00 | 0.50 | 1SG | 973 |
| ATOM | 973 | ND1 | HIS | 119 | −7.323 | 10.282 | 11.221 | 1.00 | 0.50 | 1SG | 974 |
| ATOM | 974 | CG | HIS | 119 | −6.722 | 10.533 | 12.431 | 1.00 | 0.50 | 1SG | 975 |
| ATOM | 975 | CB | HIS | 119 | −5.236 | 10.569 | 12.618 | 1.00 | 0.50 | 1SG | 976 |
| ATOM | 976 | NE2 | HIS | 119 | −8.963 | 10.653 | 12.669 | 1.00 | 0.50 | 1SG | 977 |
| ATOM | 977 | CD2 | HIS | 119 | −7.741 | 10.755 | 13.308 | 1.00 | 0.50 | 1SG | 978 |
| ATOM | 978 | CE1 | HIS | 119 | −8.660 | 10.367 | 11.417 | 1.00 | 0.50 | 1SG | 979 |
| ATOM | 979 | C | HIS | 119 | −3.092 | 9.517 | 12.404 | 1.00 | 0.50 | 1SG | 980 |
| ATOM | 980 | O | HIS | 119 | −2.514 | 10.104 | 13.300 | 1.00 | 0.50 | 1SG | 981 |
| ATOM | 981 | N | TYR | 120 | −2.468 | 9.117 | 11.283 | 1.00 | 0.75 | 1SG | 982 |
| ATOM | 982 | CA | TYR | 120 | −1.151 | 9.560 | 10.913 | 1.00 | 0.75 | 1SG | 983 |
| ATOM | 983 | CB | TYR | 120 | −0.192 | 10.192 | 11.924 | 1.00 | 0.75 | 1SG | 984 |
| ATOM | 984 | CG | TYR | 120 | −0.551 | 11.634 | 12.093 | 1.00 | 0.75 | 1SG | 985 |
| ATOM | 985 | CD1 | TYR | 120 | −0.574 | 12.454 | 10.989 | 1.00 | 0.75 | 1SG | 986 |
| ATOM | 986 | CD2 | TYR | 120 | −0.792 | 12.212 | 13.322 | 1.00 | 0.75 | 1SG | 987 |
| ATOM | 987 | CE1 | TYR | 120 | −0.880 | 13.791 | 11.099 | 1.00 | 0.75 | 1SG | 988 |
| ATOM | 988 | CE2 | TYR | 120 | −1.108 | 13.549 | 13.450 | 1.00 | 0.75 | 1SG | 989 |
| ATOM | 989 | CZ | TYR | 120 | −1.162 | 14.340 | 12.323 | 1.00 | 0.75 | 1SG | 990 |
| ATOM | 990 | OH | TYR | 120 | −1.473 | 15.710 | 12.400 | 1.00 | 0.75 | 1SG | 991 |
| ATOM | 991 | C | TYR | 120 | −0.367 | 8.641 | 10.059 | 1.00 | 0.75 | 1SG | 992 |
| ATOM | 992 | O | TYR | 120 | −0.848 | 7.689 | 9.456 | 1.00 | 0.75 | 1SG | 993 |
| ATOM | 993 | N | ASP | 121 | 0.911 | 9.017 | 9.946 | 1.00 | 1.06 | 1SG | 994 |
| ATOM | 994 | CA | ASP | 121 | 1.849 | 8.338 | 9.132 | 1.00 | 1.06 | 1SG | 995 |
| ATOM | 995 | CB | ASP | 121 | 1.306 | 7.982 | 7.731 | 1.00 | 1.06 | 1SG | 996 |
| ATOM | 996 | CG | ASP | 121 | 2.119 | 6.833 | 7.139 | 1.00 | 1.06 | 1SG | 997 |
| ATOM | 997 | OD1 | ASP | 121 | 2.635 | 5.999 | 7.931 | 1.00 | 1.06 | 1SG | 998 |
| ATOM | 998 | OD2 | ASP | 121 | 2.233 | 6.772 | 5.884 | 1.00 | 1.06 | 1SG | 999 |
| ATOM | 999 | C | ASP | 121 | 2.898 | 9.338 | 8.961 | 1.00 | 1.06 | 1SG | 1000 |
| ATOM | 1000 | O | ASP | 121 | 3.147 | 10.084 | 9.902 | 1.00 | 1.06 | 1SG | 1001 |
| ATOM | 1001 | N | ARG | 122 | 3.518 | 9.374 | 7.769 | 1.00 | 1.27 | 1SG | 1002 |
| ATOM | 1002 | CA | ARG | 122 | 4.511 | 10.375 | 7.560 | 1.00 | 1.27 | 1SG | 1003 |
| ATOM | 1003 | CB | ARG | 122 | 4.010 | 11.786 | 7.945 | 1.00 | 1.27 | 1SG | 1004 |
| ATOM | 1004 | CG | ARG | 122 | 5.101 | 12.831 | 8.209 | 1.00 | 1.27 | 1SG | 1005 |
| ATOM | 1005 | CD | ARG | 122 | 4.550 | 14.147 | 8.766 | 1.00 | 1.27 | 1SG | 1006 |
| ATOM | 1006 | NE | ARG | 122 | 5.633 | 14.870 | 9.501 | 1.00 | 1.27 | 1SG | 1007 |
| ATOM | 1007 | CZ | ARG | 122 | 6.528 | 15.681 | 8.859 | 1.00 | 1.27 | 1SG | 1008 |
| ATOM | 1008 | NH1 | ARG | 122 | 6.537 | 15.757 | 7.496 | 1.00 | 1.27 | 1SG | 1009 |
| ATOM | 1009 | NH2 | ARG | 122 | 7.433 | 16.405 | 9.581 | 1.00 | 1.27 | 1SG | 1010 |
| ATOM | 1010 | C | ARG | 122 | 5.728 | 10.082 | 8.380 | 1.00 | 1.27 | 1SG | 1011 |
| ATOM | 1011 | O | ARG | 122 | 6.708 | 9.524 | 7.907 | 1.00 | 1.27 | 1SG | 1012 |
| ATOM | 1012 | N | LYS | 123 | 5.707 | 10.483 | 9.655 | 1.00 | 1.09 | 1SG | 1013 |
| ATOM | 1013 | CA | LYS | 123 | 6.852 | 10.432 | 10.510 | 1.00 | 1.09 | 1SG | 1014 |
| ATOM | 1014 | CB | LYS | 123 | 6.738 | 11.377 | 11.713 | 1.00 | 1.09 | 1SG | 1015 |
| ATOM | 1015 | CG | LYS | 123 | 5.545 | 11.112 | 12.623 | 1.00 | 1.09 | 1SG | 1016 |
| ATOM | 1016 | CD | LYS | 123 | 5.592 | 11.969 | 13.889 | 1.00 | 1.09 | 1SG | 1017 |
| ATOM | 1017 | CE | LYS | 123 | 5.414 | 13.466 | 13.621 | 1.00 | 1.09 | 1SG | 1018 |
| ATOM | 1018 | NZ | LYS | 123 | 3.986 | 13.782 | 13.386 | 1.00 | 1.09 | 1SG | 1019 |
| ATOM | 1019 | C | LYS | 123 | 7.295 | 9.069 | 10.973 | 1.00 | 1.09 | 1SG | 1020 |
| ATOM | 1020 | O | LYS | 123 | 8.436 | 8.973 | 11.414 | 1.00 | 1.09 | 1SG | 1021 |
| ATOM | 1021 | N | ALA | 124 | 6.451 | 8.007 | 10.978 | 1.00 | 0.85 | 1SG | 1022 |
| ATOM | 1022 | CA | ALA | 124 | 6.965 | 6.754 | 11.507 | 1.00 | 0.85 | 1SG | 1023 |
| ATOM | 1023 | CB | ALA | 124 | 6.013 | 6.055 | 12.494 | 1.00 | 0.85 | 1SG | 1024 |
| ATOM | 1024 | C | ALA | 124 | 7.239 | 5.769 | 10.399 | 1.00 | 0.85 | 1SG | 1025 |
| ATOM | 1025 | O | ALA | 124 | 6.321 | 5.100 | 9.930 | 1.00 | 0.85 | 1SG | 1026 |
| ATOM | 1026 | N | PRO | 125 | 8.514 | 5.562 | 10.102 | 1.00 | 0.92 | 1SG | 1027 |
| ATOM | 1027 | CA | PRO | 125 | 8.965 | 4.789 | 8.962 | 1.00 | 0.92 | 1SG | 1028 |
| ATOM | 1028 | CD | PRO | 125 | 9.488 | 6.590 | 10.433 | 1.00 | 0.92 | 1SG | 1029 |
| ATOM | 1029 | CB | PRO | 125 | 10.410 | 5.207 | 8.697 | 1.00 | 0.92 | 1SG | 1030 |
| ATOM | 1030 | CG | PRO | 125 | 10.496 | 6.618 | 9.285 | 1.00 | 0.92 | 1SG | 1031 |
| ATOM | 1031 | C | PRO | 125 | 8.823 | 3.299 | 8.909 | 1.00 | 0.92 | 1SG | 1032 |
| ATOM | 1032 | O | PRO | 125 | 8.272 | 2.674 | 9.815 | 1.00 | 0.92 | 1SG | 1033 |
| ATOM | 1033 | N | ASP | 126 | 9.382 | 2.759 | 7.805 | 1.00 | 0.78 | 1SG | 1034 |
| ATOM | 1034 | CA | ASP | 126 | 9.370 | 1.429 | 7.265 | 1.00 | 0.78 | 1SG | 1035 |
| ATOM | 1035 | CB | ASP | 126 | 10.099 | 1.396 | 5.908 | 1.00 | 0.78 | 1SG | 1036 |
| ATOM | 1036 | CG | ASP | 126 | 10.007 | 0.017 | 5.263 | 1.00 | 0.78 | 1SG | 1037 |
| ATOM | 1037 | OD1 | ASP | 126 | 9.333 | −0.883 | 5.830 | 1.00 | 0.78 | 1SG | 1038 |
| ATOM | 1038 | OD2 | ASP | 126 | 10.623 | −0.151 | 4.177 | 1.00 | 0.78 | 1SG | 1039 |
| ATOM | 1039 | C | ASP | 126 | 10.039 | 0.421 | 8.140 | 1.00 | 0.78 | 1SG | 1040 |
| ATOM | 1040 | O | ASP | 126 | 9.469 | −0.648 | 8.355 | 1.00 | 0.78 | 1SG | 1041 |
| ATOM | 1041 | N | THR | 127 | 11.251 | 0.722 | 8.662 | 1.00 | 0.40 | 1SG | 1042 |
| ATOM | 1042 | CA | THR | 127 | 12.024 | −0.259 | 9.383 | 1.00 | 0.40 | 1SG | 1043 |
| ATOM | 1043 | CB | THR | 127 | 13.254 | 0.323 | 10.017 | 1.00 | 0.40 | 1SG | 1044 |
| ATOM | 1044 | OG1 | THR | 127 | 14.084 | 0.908 | 9.022 | 1.00 | 0.40 | 1SG | 1045 |
| ATOM | 1045 | CG2 | THR | 127 | 14.019 | −0.799 | 10.745 | 1.00 | 0.40 | 1SG | 1046 |
| ATOM | 1046 | C | THR | 127 | 11.175 | −0.845 | 10.458 | 1.00 | 0.40 | 1SG | 1047 |
| ATOM | 1047 | O | THR | 127 | 10.777 | −2.006 | 10.377 | 1.00 | 0.40 | 1SG | 1048 |
| ATOM | 1048 | N | PHE | 128 | 10.847 | −0.049 | 11.484 | 1.00 | 0.43 | 1SG | 1049 |
| ATOM | 1049 | CA | PHE | 128 | 9.939 | −0.548 | 12.464 | 1.00 | 0.43 | 1SG | 1050 |
| ATOM | 1050 | CB | PHE | 128 | 10.617 | −1.358 | 13.588 | 1.00 | 0.43 | 1SG | 1051 |
| ATOM | 1051 | CG | PHE | 128 | 11.189 | −2.612 | 13.016 | 1.00 | 0.43 | 1SG | 1052 |
| ATOM | 1052 | CD1 | PHE | 128 | 12.446 | −2.616 | 12.455 | 1.00 | 0.43 | 1SG | 1053 |
| ATOM | 1053 | CD2 | PHE | 128 | 10.467 | −3.783 | 13.028 | 1.00 | 0.43 | 1SG | 1054 |
| ATOM | 1054 | CE1 | PHE | 128 | 12.974 | −3.769 | 11.922 | 1.00 | 0.43 | 1SG | 1055 |
| ATOM | 1055 | CE2 | PHE | 128 | 10.990 | −4.940 | 12.498 | 1.00 | 0.43 | 1SG | 1056 |
| ATOM | 1056 | CZ | PHE | 128 | 12.246 | −4.934 | 11.943 | 1.00 | 0.43 | 1SG | 1057 |
| ATOM | 1057 | C | PHE | 128 | 9.359 | 0.654 | 13.112 | 1.00 | 0.43 | 1SG | 1058 |
| ATOM | 1058 | O | PHE | 128 | 9.627 | 0.905 | 14.287 | 1.00 | 0.43 | 1SG | 1059 |
| ATOM | 1059 | N | LYS | 129 | 8.553 | 1.453 | 12.384 | 1.00 | 0.58 | 1SG | 1060 |
| ATOM | 1060 | CA | LYS | 129 | 8.070 | 2.556 | 13.137 | 1.00 | 0.58 | 1SG | 1061 |
| ATOM | 1061 | CB | LYS | 129 | 8.221 | 3.883 | 12.412 | 1.00 | 0.58 | 1SG | 1062 |
| ATOM | 1062 | CG | LYS | 129 | 9.707 | 4.229 | 12.288 | 1.00 | 0.58 | 1SG | 1063 |
| ATOM | 1063 | CD | LYS | 129 | 10.471 | 3.303 | 11.335 | 1.00 | 0.58 | 1SG | 1064 |
| ATOM | 1064 | CE | LYS | 129 | 11.990 | 3.383 | 11.458 | 1.00 | 0.58 | 1SG | 1065 |
| ATOM | 1065 | NZ | LYS | 129 | 12.614 | 3.155 | 10.135 | 1.00 | 0.58 | 1SG | 1066 |
| ATOM | 1066 | C | LYS | 129 | 6.679 | 2.287 | 13.581 | 1.00 | 0.58 | 1SG | 1067 |
| ATOM | 1067 | O | LYS | 129 | 5.975 | 3.181 | 14.042 | 1.00 | 0.58 | 1SG | 1068 |
| ATOM | 1068 | N | VAL | 130 | 6.281 | 1.008 | 13.501 | 1.00 | 0.37 | 1SG | 1069 |
| ATOM | 1069 | CA | VAL | 130 | 5.059 | 0.526 | 14.068 | 1.00 | 0.37 | 1SG | 1070 |
| ATOM | 1070 | CB | VAL | 130 | 4.017 | 0.124 | 13.066 | 1.00 | 0.37 | 1SG | 1071 |
| ATOM | 1071 | CG1 | VAL | 130 | 2.907 | −0.632 | 13.813 | 1.00 | 0.37 | 1SG | 1072 |
| ATOM | 1072 | CG2 | VAL | 130 | 3.494 | 1.390 | 12.368 | 1.00 | 0.37 | 1SG | 1073 |
| ATOM | 1073 | C | VAL | 130 | 5.496 | −0.718 | 14.768 | 1.00 | 0.37 | 1SG | 1074 |
| ATOM | 1074 | O | VAL | 130 | 6.210 | −1.532 | 14.185 | 1.00 | 0.37 | 1SG | 1075 |
| ATOM | 1075 | N | ALA | 131 | 5.105 | −0.893 | 16.043 | 1.00 | 0.16 | 1SG | 1076 |
| ATOM | 1076 | CA | ALA | 131 | 5.552 | −2.049 | 16.768 | 1.00 | 0.16 | 1SG | 1077 |
| ATOM | 1077 | CB | ALA | 131 | 5.355 | −1.949 | 18.288 | 1.00 | 0.16 | 1SG | 1078 |
| ATOM | 1078 | C | ALA | 131 | 4.823 | −3.266 | 16.280 | 1.00 | 0.16 | 1SG | 1079 |
| ATOM | 1079 | O | ALA | 131 | 3.724 | −3.174 | 15.734 | 1.00 | 0.16 | 1SG | 1080 |
| ATOM | 1080 | N | MET | 132 | 5.462 | −4.445 | 16.453 | 1.00 | 0.28 | 1SG | 1081 |
| ATOM | 1081 | CA | MET | 132 | 4.947 | −5.723 | 16.035 | 1.00 | 0.28 | 1SG | 1082 |
| ATOM | 1082 | CB | MET | 132 | 5.984 | −6.860 | 16.078 | 1.00 | 0.28 | 1SG | 1083 |
| ATOM | 1083 | CG | MET | 132 | 7.054 | −6.722 | 14.990 | 1.00 | 0.28 | 1SG | 1084 |
| ATOM | 1084 | SD | MET | 132 | 8.084 | −8.197 | 14.722 | 1.00 | 0.28 | 1SG | 1085 |
| ATOM | 1085 | CE | MET | 132 | 9.156 | −7.927 | 16.162 | 1.00 | 0.28 | 1SG | 1086 |
| ATOM | 1086 | C | MET | 132 | 3.762 | −6.124 | 16.851 | 1.00 | 0.28 | 1SG | 1087 |
| ATOM | 1087 | O | MET | 132 | 2.807 | −6.692 | 16.322 | 1.00 | 0.28 | 1SG | 1088 |
| ATOM | 1088 | N | ASN | 133 | 3.757 | −5.783 | 18.159 | 1.00 | 0.53 | 1SG | 1089 |
| ATOM | 1089 | CA | ASN | 133 | 2.676 | −6.104 | 19.057 | 1.00 | 0.53 | 1SG | 1090 |
| ATOM | 1090 | CB | ASN | 133 | 2.920 | −5.633 | 20.504 | 1.00 | 0.53 | 1SG | 1091 |
| ATOM | 1091 | CG | ASN | 133 | 3.116 | −4.126 | 20.527 | 1.00 | 0.53 | 1SG | 1092 |
| ATOM | 1092 | OD1 | ASN | 133 | 3.461 | −3.506 | 19.523 | 1.00 | 0.53 | 1SG | 1093 |
| ATOM | 1093 | ND2 | ASN | 133 | 2.913 | −3.516 | 21.725 | 1.00 | 0.53 | 1SG | 1094 |
| ATOM | 1094 | C | ASN | 133 | 1.484 | −5.445 | 18.497 | 1.00 | 0.53 | 1SG | 1095 |
| ATOM | 1095 | O | ASN | 133 | 0.338 | −5.772 | 18.803 | 1.00 | 0.53 | 1SG | 1096 |
| ATOM | 1096 | N | ASN | 134 | 1.769 | −4.398 | 17.730 | 1.00 | 0.72 | 1SG | 1097 |
| ATOM | 1097 | CA | ASN | 134 | 0.790 | −3.811 | 16.915 | 1.00 | 0.72 | 1SG | 1098 |
| ATOM | 1098 | CB | ASN | 134 | −0.256 | −4.783 | 16.329 | 1.00 | 0.72 | 1SG | 1099 |
| ATOM | 1099 | CG | ASN | 134 | 0.445 | −5.681 | 15.314 | 1.00 | 0.72 | 1SG | 1100 |
| ATOM | 1100 | OD1 | ASN | 134 | 1.439 | −5.289 | 14.704 | 1.00 | 0.72 | 1SG | 1101 |
| ATOM | 1101 | ND2 | ASN | 134 | −0.089 | −6.918 | 15.123 | 1.00 | 0.72 | 1SG | 1102 |
| ATOM | 1102 | C | ASN | 134 | 0.128 | −2.777 | 17.695 | 1.00 | 0.72 | 1SG | 1103 |
| ATOM | 1103 | O | ASN | 134 | −0.275 | −2.944 | 18.845 | 1.00 | 0.72 | 1SG | 1104 |
| ATOM | 1104 | N | LEU | 135 | 0.069 | −1.645 | 17.018 | 1.00 | 0.68 | 1SG | 1105 |
| ATOM | 1105 | CA | LEU | 135 | −0.645 | −0.501 | 17.411 | 1.00 | 0.68 | 1SG | 1106 |
| ATOM | 1106 | CB | LEU | 135 | −0.505 | 0.542 | 16.281 | 1.00 | 0.68 | 1SG | 1107 |
| ATOM | 1107 | CG | LEU | 135 | −1.429 | 1.773 | 16.249 | 1.00 | 0.68 | 13G | 1108 |
| ATOM | 1108 | CD2 | LEU | 135 | −2.903 | 1.401 | 16.011 | 1.00 | 0.68 | 1SG | 1109 |
| ATOM | 1109 | CD1 | LEU | 135 | −0.958 | 2.722 | 15.136 | 1.00 | 0.68 | 1SG | 1110 |
| ATOM | 1110 | C | LEU | 135 | −2.040 | −1.001 | 17.461 | 1.00 | 0.68 | 1SG | 1111 |
| ATOM | 1111 | O | LEU | 135 | −2.819 | −0.653 | 18.346 | 1.00 | 0.68 | 1SG | 1112 |
| ATOM | 1112 | N | ALA | 136 | −2.358 | −1.882 | 16.496 | 1.00 | 0.41 | 1SG | 1113 |
| ATOM | 1113 | CA | ALA | 136 | −3.677 | −2.413 | 16.333 | 1.00 | 0.41 | 1SG | 1114 |
| ATOM | 1114 | CB | ALA | 136 | −3.795 | −3.346 | 15.111 | 1.00 | 0.41 | 1SG | 1115 |
| ATOM | 1115 | C | ALA | 136 | −4.128 | −3.206 | 17.521 | 1.00 | 0.41 | 1SG | 1116 |
| ATOM | 1116 | O | ALA | 136 | −5.229 | −2.990 | 18.026 | 1.00 | 0.41 | 1SG | 1117 |
| ATOM | 1117 | N | LYS | 137 | −3.303 | −4.150 | 18.008 | 1.00 | 0.36 | 1SG | 1118 |
| ATOM | 1118 | CA | LYS | 137 | −3.782 | −4.997 | 19.059 | 1.00 | 0.36 | 1SG | 1119 |
| ATOM | 1119 | CB | LYS | 137 | −2.854 | −6.200 | 19.301 | 1.00 | 0.36 | 1SG | 1120 |
| ATOM | 1120 | CG | LYS | 137 | −3.543 | −7.403 | 19.953 | 1.00 | 0.36 | 1SG | 1121 |
| ATOM | 1121 | CD | LYS | 137 | −2.841 | −8.739 | 19.674 | 1.00 | 0.36 | 1SG | 1122 |
| ATOM | 1122 | CE | LYS | 137 | −3.170 | −9.305 | 18.282 | 1.00 | 0.36 | 1SG | 1123 |
| ATOM | 1123 | NZ | LYS | 137 | −2.288 | −10.459 | 17.969 | 1.00 | 0.36 | 1SG | 1124 |
| ATOM | 1124 | C | LYS | 137 | −3.949 | −4.164 | 20.285 | 1.00 | 0.36 | 1SG | 1125 |
| ATOM | 1125 | O | LYS | 137 | −4.897 | −4.338 | 21.046 | 1.00 | 0.36 | 1SG | 1126 |
| ATOM | 1126 | N | CYS | 138 | −3.016 | −3.226 | 20.514 | 1.00 | 0.51 | 1SG | 1127 |
| ATOM | 1127 | CA | CYS | 138 | −3.110 | −2.371 | 21.660 | 1.00 | 0.51 | 1SG | 1128 |
| ATOM | 1128 | CB | CYS | 138 | −1.848 | −1.518 | 21.823 | 1.00 | 0.51 | 1SG | 1129 |
| ATOM | 1129 | SG | CYS | 138 | −0.363 | −2.539 | 22.079 | 1.00 | 0.51 | 1SG | 1130 |
| ATOM | 1130 | C | CYS | 138 | −4.311 | −1.476 | 21.525 | 1.00 | 0.51 | 1SG | 1131 |
| ATOM | 1131 | O | CYS | 138 | −5.079 | −1.310 | 22.474 | 1.00 | 0.51 | 1SG | 1132 |
| ATOM | 1132 | N | PHE | 139 | −4.520 | −0.888 | 20.332 | 1.00 | 0.47 | 1SG | 1133 |
| ATOM | 1133 | CA | PHE | 139 | −5.651 | −0.021 | 20.174 | 1.00 | 0.47 | 1SG | 1134 |
| ATOM | 1134 | CB | PHE | 139 | −5.655 | 0.821 | 18.881 | 1.00 | 0.47 | 1SG | 1135 |
| ATOM | 1135 | CG | PHE | 139 | −4.870 | 2.066 | 19.146 | 1.00 | 0.47 | 1SG | 1136 |
| ATOM | 1136 | CD1 | PHE | 139 | −5.485 | 3.127 | 19.772 | 1.00 | 0.47 | 1SG | 1137 |
| ATOM | 1137 | CD2 | PHE | 139 | −3.554 | 2.206 | 18.769 | 1.00 | 0.47 | 1SG | 1138 |
| ATOM | 1138 | CE1 | PHE | 139 | −4.808 | 4.295 | 20.033 | 1.00 | 0.47 | 1SG | 1139 |
| ATOM | 1139 | CE2 | PHE | 139 | −2.870 | 3.374 | 19.028 | 1.00 | 0.47 | 1SG | 1140 |
| ATOM | 1140 | CZ | PHE | 139 | −3.491 | 4.423 | 19.663 | 1.00 | 0.47 | 1SG | 1141 |
| ATOM | 1141 | C | PHE | 139 | −6.925 | −0.806 | 20.262 | 1.00 | 0.47 | 1SG | 1142 |
| ATOM | 1142 | O | PHE | 139 | −7.947 | −0.283 | 20.705 | 1.00 | 0.47 | 1SG | 1143 |
| ATOM | 1143 | N | SER | 140 | −6.911 | −2.083 | 19.829 | 1.00 | 0.33 | 1SG | 1144 |
| ATOM | 1144 | CA | SER | 140 | −8.131 | −2.839 | 19.788 | 1.00 | 0.33 | 1SG | 1145 |
| ATOM | 1145 | CB | SER | 140 | −7.973 | −4.246 | 19.165 | 1.00 | 0.33 | 1SG | 1146 |
| ATOM | 1146 | OG | SER | 140 | −7.467 | −5.159 | 20.124 | 1.00 | 0.33 | 1SG | 1147 |
| ATOM | 1147 | C | SER | 140 | −8.717 | −3.056 | 21.153 | 1.00 | 0.33 | 1SG | 1148 |
| ATOM | 1148 | O | SER | 140 | −9.925 | −2.904 | 21.332 | 1.00 | 0.33 | 1SG | 1149 |
| ATOM | 1149 | N | ASN | 141 | −7.903 | −3.472 | 22.149 | 1.00 | 0.45 | 1SG | 1150 |
| ATOM | 1150 | CA | ASN | 141 | −8.495 | −3.697 | 23.440 | 1.00 | 0.45 | 1SG | 1151 |
| ATOM | 1151 | CB | ASN | 141 | −7.911 | −4.819 | 24.311 | 1.00 | 0.45 | 1SG | 1152 |
| ATOM | 1152 | CG | ASN | 141 | −8.829 | −5.987 | 23.960 | 1.00 | 0.45 | 1SG | 1153 |
| ATOM | 1153 | OD1 | ASN | 141 | −8.535 | −6.881 | 23.172 | 1.00 | 0.45 | 1SG | 1154 |
| ATOM | 1154 | ND2 | ASN | 141 | −10.054 | −5.947 | 24.544 | 1.00 | 0.45 | 1SG | 1155 |
| ATOM | 1155 | C | ASN | 141 | −8.742 | −2.474 | 24.241 | 1.00 | 0.45 | 1SG | 1156 |
| ATOM | 1156 | O | ASN | 141 | −9.631 | −2.486 | 25.094 | 1.00 | 0.45 | 1SG | 1157 |
| ATOM | 1157 | N | ILE | 142 | −7.946 | −1.409 | 24.038 | 1.00 | 0.75 | 1SG | 1158 |
| ATOM | 1158 | CA | ILE | 142 | −8.111 | −0.214 | 24.806 | 1.00 | 0.75 | 1SG | 1159 |
| ATOM | 1159 | CB | ILE | 142 | −6.847 | 0.606 | 25.003 | 1.00 | 0.75 | 1SG | 1160 |
| ATOM | 1160 | CG2 | ILE | 142 | −6.479 | 1.358 | 23.721 | 1.00 | 0.75 | 1SG | 1161 |
| ATOM | 1161 | CG1 | ILE | 142 | −7.064 | 1.520 | 26.219 | 1.00 | 0.75 | 1SG | 1162 |
| ATOM | 1162 | CD1 | ILE | 142 | −7.416 | 0.711 | 27.473 | 1.00 | 0.75 | 1SG | 1163 |
| ATOM | 1163 | C | ILE | 142 | −9.291 | 0.627 | 24.379 | 1.00 | 0.75 | 1SG | 1164 |
| ATOM | 1164 | O | ILE | 142 | −9.797 | 1.421 | 25.174 | 1.00 | 0.75 | 1SG | 1165 |
| ATOM | 1165 | N | PHE | 143 | −9.747 | 0.490 | 23.118 | 1.00 | 0.70 | 1SG | 1166 |
| ATOM | 1166 | CA | PHE | 143 | −10.727 | 1.383 | 22.564 | 1.00 | 0.70 | 1SG | 1167 |
| ATOM | 1167 | CB | PHE | 143 | −11.281 | 1.101 | 21.130 | 1.00 | 0.70 | 1SG | 1168 |
| ATOM | 1168 | CG | PHE | 143 | −12.568 | 0.323 | 21.156 | 1.00 | 0.70 | 1SG | 1169 |
| ATOM | 1169 | CD1 | PHE | 143 | −12.596 | −1.045 | 21.163 | 1.00 | 0.70 | 1SG | 1170 |
| ATOM | 1170 | CD2 | PHE | 143 | −13.772 | 1.006 | 21.161 | 1.00 | 0.70 | 1SG | 1171 |
| ATOM | 1171 | CE1 | PHE | 143 | −13.799 | −1.712 | 21.188 | 1.00 | 0.70 | 1SG | 1172 |
| ATOM | 1172 | CE2 | PHE | 143 | −14.977 | 0.346 | 21.192 | 1.00 | 0.70 | 1SG | 1173 |
| ATOM | 1173 | CZ | PHE | 143 | −14.990 | −1.027 | 21.205 | 1.00 | 0.70 | 1SG | 1174 |
| ATOM | 1174 | C | PHE | 143 | −11.930 | 1.284 | 23.446 | 1.00 | 0.70 | 1SG | 1175 |
| ATOM | 1175 | O | PHE | 143 | −12.604 | 2.279 | 23.708 | 1.00 | 0.70 | 1SG | 1176 |
| ATOM | 1176 | N | ILE | 144 | −12.220 | 0.071 | 23.953 | 1.00 | 0.30 | 1SG | 1177 |
| ATOM | 1177 | CA | ILE | 144 | −13.363 | −0.096 | 24.797 | 1.00 | 0.30 | 1SG | 1178 |
| ATOM | 1178 | CB | ILE | 144 | −13.546 | −1.505 | 25.286 | 1.00 | 0.30 | 1SG | 1179 |
| ATOM | 1179 | CG2 | ILE | 144 | −14.567 | −1.484 | 26.431 | 1.00 | 0.30 | 1SG | 1180 |
| ATOM | 1180 | CG1 | ILE | 144 | −13.946 | −2.431 | 24.120 | 1.00 | 0.30 | 1SG | 1181 |
| ATOM | 1181 | CD1 | ILE | 144 | −14.069 | −3.907 | 24.501 | 1.00 | 0.30 | 1SG | 1182 |
| ATOM | 1182 | C | ILE | 144 | −13.202 | 0.788 | 25.995 | 1.00 | 0.30 | 1SG | 1183 |
| ATOM | 1183 | O | ILE | 144 | −14.174 | 1.376 | 26.465 | 1.00 | 0.30 | 1SG | 1184 |
| ATOM | 1184 | N | ALA | 145 | −11.973 | 0.902 | 26.535 | 1.00 | 0.09 | 1SG | 1185 |
| ATOM | 1185 | CA | ALA | 145 | −11.757 | 1.722 | 27.692 | 1.00 | 0.09 | 1SG | 1186 |
| ATOM | 1186 | CB | ALA | 145 | −10.312 | 1.659 | 28.224 | 1.00 | 0.09 | 1SG | 1187 |
| ATOM | 1187 | C | ALA | 145 | −12.043 | 3.148 | 27.335 | 1.00 | 0.09 | 1SG | 1188 |
| ATOM | 1188 | O | ALA | 145 | −12.677 | 3.866 | 28.105 | 1.00 | 0.09 | 1SG | 1189 |
| ATOM | 1189 | N | SER | 146 | −11.596 | 3.600 | 26.149 | 1.00 | 0.32 | 1SG | 1190 |
| ATOM | 1190 | CA | SER | 146 | −11.819 | 4.970 | 25.778 | 1.00 | 0.32 | 1SG | 1191 |
| ATOM | 1191 | CB | SER | 146 | −11.208 | 5.351 | 24.414 | 1.00 | 0.32 | 1SG | 1192 |
| ATOM | 1192 | OG | SER | 146 | −11.922 | 4.741 | 23.352 | 1.00 | 0.32 | 1SG | 1193 |
| ATOM | 1193 | C | SER | 146 | −13.291 | 5.171 | 25.667 | 1.00 | 0.32 | 1SG | 1194 |
| ATOM | 1194 | O | SER | 146 | −13.831 | 6.196 | 26.082 | 1.00 | 0.32 | 1SG | 1195 |
| ATOM | 1195 | N | LYS | 147 | −13.975 | 4.166 | 25.104 | 1.00 | 0.74 | 1SG | 1196 |
| ATOM | 1196 | CA | LYS | 147 | −15.376 | 4.233 | 24.848 | 1.00 | 0.74 | 1SG | 1197 |
| ATOM | 1197 | CB | LYS | 147 | −15.829 | 2.898 | 24.227 | 1.00 | 0.74 | 1SG | 1198 |
| ATOM | 1198 | CG | LYS | 147 | −17.122 | 2.903 | 23.416 | 1.00 | 0.74 | 1SG | 1199 |
| ATOM | 1199 | CD | LYS | 147 | −18.387 | 3.211 | 24.204 | 1.00 | 0.74 | 1SG | 1200 |
| ATOM | 1200 | CE | LYS | 147 | −19.638 | 2.800 | 23.431 | 1.00 | 0.74 | 1SG | 1201 |
| ATOM | 1201 | NZ | LYS | 147 | −19.571 | 3.330 | 22.051 | 1.00 | 0.74 | 1SG | 1202 |
| ATOM | 1202 | C | LYS | 147 | −16.091 | 4.440 | 26.151 | 1.00 | 0.74 | 1SG | 1203 |
| ATOM | 1203 | O | LYS | 147 | −16.917 | 5.343 | 26.272 | 1.00 | 0.74 | 1SG | 1204 |
| ATOM | 1204 | N | LEU | 148 | −15.777 | 3.615 | 27.172 | 1.00 | 0.83 | 1SG | 1205 |
| ATOM | 1205 | CA | LEU | 148 | −16.488 | 3.678 | 28.421 | 1.00 | 0.83 | 1SG | 1206 |
| ATOM | 1206 | CB | LEU | 148 | −16.254 | 2.439 | 29.309 | 1.00 | 0.83 | 1SG | 1207 |
| ATOM | 1207 | CG | LEU | 148 | −17.194 | 2.341 | 30.532 | 1.00 | 0.83 | 1SG | 1208 |
| ATOM | 1208 | CD2 | LEU | 148 | −18.664 | 2.399 | 30.087 | 1.00 | 0.83 | 1SG | 1209 |
| ATOM | 1209 | CD1 | LEU | 148 | −16.868 | 3.371 | 31.626 | 1.00 | 0.83 | 1SG | 1210 |
| ATOM | 1210 | C | LEU | 148 | −16.161 | 4.907 | 29.222 | 1.00 | 0.83 | 1SG | 1211 |
| ATOM | 1211 | O | LEU | 148 | −17.063 | 5.574 | 29.726 | 1.00 | 0.83 | 1SG | 1212 |
| ATOM | 1212 | N | GLU | 149 | −14.871 | 5.266 | 29.370 | 1.00 | 0.56 | 1SG | 1213 |
| ATOM | 1213 | CA | GLU | 149 | −14.609 | 6.363 | 30.264 | 1.00 | 0.56 | 1SG | 1214 |
| ATOM | 1214 | CB | GLU | 149 | −13.408 | 6.142 | 31.197 | 1.00 | 0.56 | 1SG | 1215 |
| ATOM | 1215 | CG | GLU | 149 | −13.696 | 5.152 | 32.325 | 1.00 | 0.56 | 1SG | 1216 |
| ATOM | 1216 | CD | GLU | 149 | −14.692 | 5.808 | 33.275 | 1.00 | 0.56 | 1SG | 1217 |
| ATOM | 1217 | OE1 | GLU | 149 | −15.923 | 5.641 | 33.059 | 1.00 | 0.56 | 1SG | 1218 |
| ATOM | 1218 | OE2 | GLU | 149 | −14.231 | 6.488 | 34.230 | 1.00 | 0.56 | 1SG | 1219 |
| ATOM | 1219 | C | GLU | 149 | −14.346 | 7.614 | 29.498 | 1.00 | 0.56 | 1SG | 1220 |
| ATOM | 1220 | O | GLU | 149 | −13.283 | 7.787 | 28.905 | 1.00 | 0.56 | 1SG | 1221 |
| ATOM | 1221 | N | ALA | 150 | −15.330 | 8.532 | 29.499 | 1.00 | 0.36 | 1SG | 1222 |
| ATOM | 1222 | CA | ALA | 150 | −15.164 | 9.777 | 28.816 | 1.00 | 0.36 | 1SG | 1223 |
| ATOM | 1223 | CB | ALA | 150 | −16.454 | 10.611 | 28.765 | 1.00 | 0.36 | 1SG | 1224 |
| ATOM | 1224 | C | ALA | 150 | −14.148 | 10.592 | 29.547 | 1.00 | 0.36 | 1SG | 1225 |
| ATOM | 1225 | O | ALA | 150 | −13.252 | 11.175 | 28.941 | 1.00 | 0.36 | 1SG | 1226 |
| ATOM | 1226 | N | VAL | 151 | −14.273 | 10.655 | 30.886 | 1.00 | 0.54 | 1SG | 1227 |
| ATOM | 1227 | CA | VAL | 151 | −13.400 | 11.473 | 31.672 | 1.00 | 0.54 | 1SG | 1228 |
| ATOM | 1228 | CB | VAL | 151 | −13.832 | 11.559 | 33.111 | 1.00 | 0.54 | 1SG | 1229 |
| ATOM | 1229 | CG1 | VAL | 151 | −15.180 | 12.294 | 33.153 | 1.00 | 0.54 | 1SG | 1230 |
| ATOM | 1230 | CG2 | VAL | 151 | −13.912 | 10.147 | 33.720 | 1.00 | 0.54 | 1SG | 1231 |
| ATOM | 1231 | C | VAL | 151 | −12.015 | 10.938 | 31.625 | 1.00 | 0.54 | 1SG | 1232 |
| ATOM | 1232 | O | VAL | 151 | −11.061 | 11.664 | 31.348 | 1.00 | 0.54 | 1SG | 1233 |
| ATOM | 1233 | N | GLU | 152 | −11.872 | 9.626 | 31.848 | 1.00 | 0.65 | 1SG | 1234 |
| ATOM | 1234 | CA | GLU | 152 | −10.552 | 9.103 | 31.921 | 1.00 | 0.65 | 1SG | 1235 |
| ATOM | 1235 | CB | GLU | 152 | −10.473 | 7.641 | 32.389 | 1.00 | 0.65 | 1SG | 1236 |
| ATOM | 1236 | CG | GLU | 152 | −9.028 | 7.162 | 32.515 | 1.00 | 0.65 | 1SG | 1237 |
| ATOM | 1237 | CD | GLU | 152 | −8.249 | 8.154 | 33.362 | 1.00 | 0.65 | 1SG | 1238 |
| ATOM | 1238 | OE1 | GLU | 152 | −8.889 | 8.851 | 34.194 | 1.00 | 0.65 | 1SG | 1239 |
| ATOM | 1239 | OE2 | GLU | 152 | −7.003 | 8.230 | 33.183 | 1.00 | 0.65 | 1SG | 1240 |
| ATOM | 1240 | C | GLU | 152 | −9.910 | 9.239 | 30.589 | 1.00 | 0.65 | 1SG | 1241 |
| ATOM | 1241 | O | GLU | 152 | −8.698 | 9.431 | 30.507 | 1.00 | 0.65 | 1SG | 1242 |
| ATOM | 1242 | N | TYR | 153 | −10.664 | 9.138 | 29.481 | 1.00 | 0.42 | 1SG | 1243 |
| ATOM | 1243 | CA | TYR | 153 | −9.839 | 9.290 | 28.328 | 1.00 | 0.42 | 1SG | 1244 |
| ATOM | 1244 | CB | TYR | 153 | −9.323 | 7.983 | 27.707 | 1.00 | 0.42 | 1SG | 1245 |
| ATOM | 1245 | CG | TYR | 153 | −8.395 | 7.335 | 28.675 | 1.00 | 0.42 | 1SG | 1246 |
| ATOM | 1246 | CD1 | TYR | 153 | −7.132 | 7.843 | 28.898 | 1.00 | 0.42 | 1SG | 1247 |
| ATOM | 1247 | CD2 | TYR | 153 | −8.795 | 6.227 | 29.377 | 1.00 | 0.42 | 1SG | 1248 |
| ATOM | 1248 | CE1 | TYR | 153 | −6.273 | 7.249 | 29.799 | 1.00 | 0.42 | 1SG | 1249 |
| ATOM | 1249 | CE2 | TYR | 153 | −7.935 | 5.640 | 30.269 | 1.00 | 0.42 | 1SG | 1250 |
| ATOM | 1250 | CZ | TYR | 153 | −6.674 | 6.134 | 30.486 | 1.00 | 0.42 | 1SG | 1251 |
| ATOM | 1251 | OH | TYR | 153 | −5.813 | 5.505 | 31.412 | 1.00 | 0.42 | 1SG | 1252 |
| ATOM | 1252 | C | TYR | 153 | −10.498 | 10.012 | 27.216 | 1.00 | 0.42 | 1SG | 1253 |
| ATOM | 1253 | O | TYR | 153 | −11.628 | 9.719 | 26.825 | 1.00 | 0.42 | 1SG | 1254 |
| ATOM | 1254 | N | ALA | 154 | −9.784 | 11.039 | 26.724 | 1.00 | 0.42 | 1SG | 1255 |
| ATOM | 1255 | CA | ALA | 154 | −10.121 | 11.697 | 25.505 | 1.00 | 0.42 | 1SG | 1256 |
| ATOM | 1256 | CB | ALA | 154 | −9.377 | 13.031 | 25.325 | 1.00 | 0.42 | 1SG | 1257 |
| ATOM | 1257 | C | ALA | 154 | −9.673 | 10.775 | 24.417 | 1.00 | 0.42 | 1SG | 1258 |
| ATOM | 1258 | O | ALA | 154 | −10.340 | 10.627 | 23.395 | 1.00 | 0.42 | 1SG | 1259 |
| ATOM | 1259 | N | HIS | 155 | −8.502 | 10.129 | 24.616 | 1.00 | 0.66 | 1SG | 1260 |
| ATOM | 1260 | CA | HIS | 155 | −7.992 | 9.254 | 23.603 | 1.00 | 0.66 | 1SG | 1261 |
| ATOM | 1261 | ND1 | HIS | 155 | −5.999 | 11.960 | 23.087 | 1.00 | 0.66 | 1SG | 1262 |
| ATOM | 1262 | CG | HIS | 155 | −7.199 | 11.456 | 22.639 | 1.00 | 0.66 | 1SG | 1263 |
| ATOM | 1263 | CB | HIS | 155 | −7.478 | 10.009 | 22.370 | 1.00 | 0.66 | 1SG | 1264 |
| ATOM | 1264 | NE2 | HIS | 155 | −7.364 | 13.697 | 22.832 | 1.00 | 0.66 | 1SG | 1265 |
| ATOM | 1265 | CD2 | HIS | 155 | −8.022 | 12.530 | 22.491 | 1.00 | 0.66 | 1SG | 1266 |
| ATOM | 1266 | CE1 | HIS | 155 | −6.153 | 13.305 | 23.183 | 1.00 | 0.66 | 1SG | 1267 |
| ATOM | 1267 | C | HIS | 155 | −6.913 | 8.371 | 24.148 | 1.00 | 0.66 | 1SG | 1268 |
| ATOM | 1268 | O | HIS | 155 | −6.611 | 8.391 | 25.339 | 1.00 | 0.66 | 1SG | 1269 |
| ATOM | 1269 | N | ILE | 156 | −6.345 | 7.519 | 23.267 | 1.00 | 0.72 | 1SG | 1270 |
| ATOM | 1270 | CA | ILE | 156 | −5.295 | 6.620 | 23.651 | 1.00 | 0.72 | 1SG | 1271 |
| ATOM | 1271 | CB | ILE | 156 | −5.689 | 5.175 | 23.571 | 1.00 | 0.72 | 1SG | 1272 |
| ATOM | 1272 | CG2 | ILE | 156 | −4.474 | 4.328 | 23.987 | 1.00 | 0.72 | 1SG | 1273 |
| ATOM | 1273 | CG1 | ILE | 156 | −6.938 | 4.914 | 24.428 | 1.00 | 0.72 | 1SG | 1274 |
| ATOM | 1274 | CD1 | ILE | 156 | −6.758 | 5.261 | 25.903 | 1.00 | 0.72 | 1SG | 1275 |
| ATOM | 1275 | C | ILE | 156 | −4.208 | 6.812 | 22.651 | 1.00 | 0.72 | 1SG | 1276 |
| ATOM | 1276 | O | ILE | 156 | −4.477 | 6.979 | 21.462 | 1.00 | 0.72 | 1SG | 1277 |
| ATOM | 1277 | N | SER | 157 | −2.935 | 6.800 | 23.086 | 1.00 | 0.71 | 1SG | 1278 |
| ATOM | 1278 | CA | SER | 157 | −1.948 | 7.033 | 22.079 | 1.00 | 0.71 | 1SG | 1279 |
| ATOM | 1279 | CB | SER | 157 | −1.477 | 8.486 | 22.036 | 1.00 | 0.71 | 1SG | 1280 |
| ATOM | 1280 | OG | SER | 157 | −2.544 | 9.342 | 21.653 | 1.00 | 0.71 | 1SG | 1281 |
| ATOM | 1281 | C | SER | 157 | −0.757 | 6.151 | 22.287 | 1.00 | 0.71 | 1SG | 1282 |
| ATOM | 1282 | O | SER | 157 | −0.477 | 5.753 | 23.418 | 1.00 | 0.71 | 1SG | 1283 |
| ATOM | 1283 | N | ARG | 158 | −0.010 | 5.817 | 21.195 | 1.00 | 0.79 | 1SG | 1284 |
| ATOM | 1284 | CA | ARG | 158 | 1.113 | 4.951 | 21.431 | 1.00 | 0.79 | 1SG | 1285 |
| ATOM | 1285 | CB | ARG | 158 | 0.683 | 3.485 | 21.389 | 1.00 | 0.79 | 1SG | 1286 |
| ATOM | 1286 | CG | ARG | 158 | −0.288 | 3.163 | 22.522 | 1.00 | 0.79 | 1SG | 1287 |
| ATOM | 1287 | CD | ARG | 158 | −1.141 | 1.925 | 22.269 | 1.00 | 0.79 | 1SG | 1288 |
| ATOM | 1288 | NE | ARG | 158 | −0.238 | 0.842 | 21.802 | 1.00 | 0.79 | 1SG | 1289 |
| ATOM | 1289 | CZ | ARG | 158 | −0.180 | 0.564 | 20.469 | 1.00 | 0.79 | 1SG | 1290 |
| ATOM | 1290 | NH1 | ARG | 158 | −0.844 | 1.360 | 19.580 | 1.00 | 0.79 | 1SG | 1291 |
| ATOM | 1291 | NH2 | ARG | 158 | 0.505 | −0.532 | 20.036 | 1.00 | 0.79 | 1SG | 1292 |
| ATOM | 1292 | C | ARG | 158 | 2.280 | 5.075 | 20.467 | 1.00 | 0.79 | 1SG | 1293 |
| ATOM | 1293 | O | ARG | 158 | 2.830 | 4.046 | 20.102 | 1.00 | 0.79 | 1SG | 1294 |
| ATOM | 1294 | N | LEU | 159 | 2.756 | 6.275 | 20.046 | 1.00 | 0.93 | 1SG | 1295 |
| ATOM | 1295 | CA | LEU | 159 | 3.993 | 6.238 | 19.282 | 1.00 | 0.93 | 1SG | 1296 |
| ATOM | 1296 | CB | LEU | 159 | 4.592 | 7.546 | 18.748 | 1.00 | 0.93 | 1SG | 1297 |
| ATOM | 1297 | CG | LEU | 159 | 4.104 | 8.152 | 17.436 | 1.00 | 0.93 | 1SG | 1298 |
| ATOM | 1298 | CD2 | LEU | 159 | 3.785 | 7.114 | 16.356 | 1.00 | 0.93 | 1SG | 1299 |
| ATOM | 1299 | CD1 | LEU | 159 | 5.133 | 9.203 | 16.988 | 1.00 | 0.93 | 1SG | 1300 |
| ATOM | 1300 | C | LEU | 159 | 5.092 | 5.911 | 20.229 | 1.00 | 0.93 | 1SG | 1301 |
| ATOM | 1301 | O | LEU | 159 | 5.072 | 4.892 | 20.915 | 1.00 | 0.93 | 1SG | 1302 |
| ATOM | 1302 | N | GLN | 160 | 6.102 | 6.818 | 20.258 | 1.00 | 0.94 | 1SG | 1303 |
| ATOM | 1303 | CA | GLN | 160 | 7.196 | 6.735 | 21.171 | 1.00 | 0.94 | 1SG | 1304 |
| ATOM | 1304 | CB | GLN | 160 | 7.729 | 5.321 | 21.350 | 1.00 | 0.94 | 1SG | 1305 |
| ATOM | 1305 | CG | GLN | 160 | 8.257 | 4.642 | 20.106 | 1.00 | 0.94 | 1SG | 1306 |
| ATOM | 1306 | CD | GLN | 160 | 7.884 | 3.222 | 20.398 | 1.00 | 0.94 | 1SG | 1307 |
| ATOM | 1307 | OE1 | GLN | 160 | 7.971 | 2.839 | 21.560 | 1.00 | 0.94 | 1SG | 1308 |
| ATOM | 1308 | NE2 | GLN | 160 | 7.421 | 2.449 | 19.383 | 1.00 | 0.94 | 1SG | 1309 |
| ATOM | 1309 | C | GLN | 160 | 8.311 | 7.646 | 20.767 | 1.00 | 0.94 | 1SG | 1310 |
| ATOM | 1310 | O | GLN | 160 | 8.540 | 8.664 | 21.413 | 1.00 | 0.94 | 1SG | 1311 |
| ATOM | 1311 | N | ALA | 161 | 9.028 | 7.323 | 19.676 | 1.00 | 0.64 | 1SG | 1312 |
| ATOM | 1312 | CA | ALA | 161 | 10.245 | 8.018 | 19.365 | 1.00 | 0.64 | 1SG | 1313 |
| ATOM | 1313 | CB | ALA | 161 | 10.848 | 7.588 | 18.017 | 1.00 | 0.64 | 1SG | 1314 |
| ATOM | 1314 | C | ALA | 161 | 10.110 | 9.514 | 19.340 | 1.00 | 0.64 | 1SG | 1315 |
| ATOM | 1315 | O | ALA | 161 | 10.592 | 10.162 | 20.266 | 1.00 | 0.64 | 1SG | 1316 |
| ATOM | 1316 | N | ASP | 162 | 9.465 | 10.119 | 18.319 | 1.00 | 0.57 | 1SG | 1317 |
| ATOM | 1317 | CA | ASP | 162 | 9.502 | 11.555 | 18.279 | 1.00 | 0.57 | 1SG | 1318 |
| ATOM | 1318 | CB | ASP | 162 | 8.826 | 12.184 | 17.049 | 1.00 | 0.57 | 1SG | 1319 |
| ATOM | 1319 | CG | ASP | 162 | 9.789 | 12.083 | 15.881 | 1.00 | 0.57 | 1SG | 1320 |
| ATOM | 1320 | OD1 | ASP | 162 | 10.891 | 11.504 | 16.072 | 1.00 | 0.57 | 1SG | 1321 |
| ATOM | 1321 | OD2 | ASP | 162 | 9.433 | 12.572 | 14.776 | 1.00 | 0.57 | 1SG | 1322 |
| ATOM | 1322 | C | ASP | 162 | 8.813 | 12.090 | 19.472 | 1.00 | 0.57 | 1SG | 1323 |
| ATOM | 1323 | O | ASP | 162 | 9.448 | 12.414 | 20.474 | 1.00 | 0.57 | 1SG | 1324 |
| ATOM | 1324 | N | LEU | 163 | 7.478 | 12.201 | 19.418 | 1.00 | 0.92 | 1SG | 1325 |
| ATOM | 1325 | CA | LEU | 163 | 6.936 | 12.621 | 20.659 | 1.00 | 0.92 | 1SG | 1326 |
| ATOM | 1326 | CB | LEU | 163 | 5.516 | 13.192 | 20.569 | 1.00 | 0.92 | 1SG | 1327 |
| ATOM | 1327 | CG | LEU | 163 | 5.475 | 14.526 | 19.804 | 1.00 | 0.92 | 1SG | 1328 |
| ATOM | 1328 | CD2 | LEU | 163 | 5.932 | 14.345 | 18.348 | 1.00 | 0.92 | 1SG | 1329 |
| ATOM | 1329 | CD1 | LEU | 163 | 6.273 | 15.609 | 20.549 | 1.00 | 0.92 | 1SG | 1330 |
| ATOM | 1330 | C | LEU | 163 | 6.953 | 11.414 | 21.518 | 1.00 | 0.92 | 1SG | 1331 |
| ATOM | 1331 | O | LEU | 163 | 7.823 | 11.292 | 22.380 | 1.00 | 0.92 | 1SG | 1332 |
| ATOM | 1332 | N | ASN | 164 | 6.027 | 10.453 | 21.252 | 1.00 | 1.14 | 1SG | 1333 |
| ATOM | 1333 | CA | ASN | 164 | 5.944 | 9.255 | 22.050 | 1.00 | 1.14 | 1SG | 1334 |
| ATOM | 1334 | CB | ASN | 164 | 6.112 | 9.512 | 23.546 | 1.00 | 1.14 | 1SG | 1335 |
| ATOM | 1335 | CG | ASN | 164 | 5.286 | 10.751 | 23.813 | 1.00 | 1.14 | 1SG | 1336 |
| ATOM | 1336 | OD1 | ASN | 164 | 4.099 | 10.853 | 23.511 | 1.00 | 1.14 | 1SG | 1337 |
| ATOM | 1337 | ND2 | ASN | 164 | 6.024 | 11.793 | 24.275 | 1.00 | 1.14 | 1SG | 1338 |
| ATOM | 1338 | C | ASN | 164 | 4.652 | 8.508 | 21.879 | 1.00 | 1.14 | 1SG | 1339 |
| ATOM | 1339 | O | ASN | 164 | 3.755 | 8.875 | 21.123 | 1.00 | 1.14 | 1SG | 1340 |
| ATOM | 1340 | N | CYS | 165 | 4.623 | 7.352 | 22.586 | 1.00 | 1.15 | 1SG | 1341 |
| ATOM | 1341 | CA | CYS | 165 | 3.603 | 6.353 | 22.718 | 1.00 | 1.15 | 1SG | 1342 |
| ATOM | 1342 | CB | CYS | 165 | 3.811 | 5.751 | 24.092 | 1.00 | 1.15 | 1SG | 1343 |
| ATOM | 1343 | SG | CYS | 165 | 5.570 | 5.371 | 24.262 | 1.00 | 1.15 | 1SG | 1344 |
| ATOM | 1344 | C | CYS | 165 | 2.615 | 7.270 | 23.027 | 1.00 | 1.15 | 1SG | 1345 |
| ATOM | 1345 | O | CYS | 165 | 1.631 | 7.480 | 22.349 | 1.00 | 1.15 | 1SG | 1346 |
| ATOM | 1346 | N | LEU | 166 | 3.089 | 7.815 | 24.115 | 1.00 | 0.95 | 1SG | 1347 |
| ATOM | 1347 | CA | LEU | 166 | 2.900 | 8.886 | 24.960 | 1.00 | 0.95 | 1SG | 1348 |
| ATOM | 1348 | CB | LEU | 166 | 1.501 | 8.990 | 25.585 | 1.00 | 0.95 | 1SG | 1349 |
| ATOM | 1349 | CG | LEU | 166 | 0.595 | 9.988 | 24.843 | 1.00 | 0.95 | 1SG | 1350 |
| ATOM | 1350 | CD2 | LEU | 166 | −0.869 | 9.854 | 25.286 | 1.00 | 0.95 | 1SG | 1351 |
| ATOM | 1351 | CD1 | LEU | 166 | 0.761 | 9.876 | 23.328 | 1.00 | 0.95 | 1SG | 1352 |
| ATOM | 1352 | C | LEU | 166 | 3.893 | 8.585 | 26.013 | 1.00 | 0.95 | 1SG | 1353 |
| ATOM | 1353 | O | LEU | 166 | 3.671 | 8.864 | 27.186 | 1.00 | 0.95 | 1SG | 1354 |
| ATOM | 1354 | N | SER | 167 | 5.025 | 7.959 | 25.631 | 1.00 | 0.51 | 1SG | 1355 |
| ATOM | 1355 | CA | SER | 167 | 5.994 | 7.841 | 26.667 | 1.00 | 0.51 | 1SG | 1356 |
| ATOM | 1356 | CB | SER | 167 | 6.636 | 6.459 | 26.889 | 1.00 | 0.51 | 1SG | 1357 |
| ATOM | 1357 | OG | SER | 167 | 7.561 | 6.505 | 27.965 | 1.00 | 0.51 | 1SG | 1358 |
| ATOM | 1358 | C | SER | 167 | 7.045 | 8.790 | 26.244 | 1.00 | 0.51 | 1SG | 1359 |
| ATOM | 1359 | O | SER | 167 | 7.949 | 8.448 | 25.484 | 1.00 | 0.51 | 1SG | 1360 |
| ATOM | 1360 | N | ASP | 168 | 6.909 | 10.036 | 26.721 | 1.00 | 0.36 | 1SG | 1361 |
| ATOM | 1361 | CA | ASP | 168 | 7.824 | 11.075 | 26.374 | 1.00 | 0.36 | 1SG | 1362 |
| ATOM | 1362 | CB | ASP | 168 | 7.562 | 12.384 | 27.145 | 1.00 | 0.36 | 1SG | 1363 |
| ATOM | 1363 | CG | ASP | 168 | 6.123 | 12.828 | 26.940 | 1.00 | 0.36 | 1SG | 1364 |
| ATOM | 1364 | OD1 | ASP | 168 | 5.216 | 11.982 | 27.167 | 1.00 | 0.36 | 1SG | 1365 |
| ATOM | 1365 | OD2 | ASP | 168 | 5.906 | 14.006 | 26.549 | 1.00 | 0.36 | 1SG | 1366 |
| ATOM | 1366 | C | ASP | 168 | 9.128 | 10.602 | 26.905 | 1.00 | 0.36 | 1SG | 1367 |
| ATOM | 1367 | O | ASP | 168 | 10.194 | 10.792 | 26.322 | 1.00 | 0.36 | 1SG | 1368 |
| ATOM | 1368 | N | LEU | 169 | 8.996 | 9.961 | 28.070 | 1.00 | 0.62 | 1SG | 1369 |
| ATOM | 1369 | CA | LEU | 169 | 9.959 | 9.389 | 28.951 | 1.00 | 0.62 | 1SG | 1370 |
| ATOM | 1370 | CB | LEU | 169 | 9.239 | 9.010 | 30.263 | 1.00 | 0.62 | 1SG | 1371 |
| ATOM | 1371 | CG | LEU | 169 | 10.044 | 8.352 | 31.396 | 1.00 | 0.62 | 1SG | 1372 |
| ATOM | 1372 | CD2 | LEU | 169 | 10.576 | 6.964 | 31.032 | 1.00 | 0.62 | 1SG | 1373 |
| ATOM | 1373 | CD1 | LEU | 169 | 9.156 | 8.281 | 32.649 | 1.00 | 0.62 | 1SG | 1374 |
| ATOM | 1374 | C | LEU | 169 | 10.565 | 8.176 | 28.313 | 1.00 | 0.62 | 1SG | 1375 |
| ATOM | 1375 | O | LEU | 169 | 11.669 | 7.783 | 28.671 | 1.00 | 0.62 | 1SG | 1376 |
| ATOM | 1376 | N | LEU | 170 | 9.892 | 7.598 | 27.301 | 1.00 | 0.92 | 1SG | 1377 |
| ATOM | 1377 | CA | LEU | 170 | 10.191 | 6.318 | 26.720 | 1.00 | 0.92 | 1SG | 1378 |
| ATOM | 1378 | CB | LEU | 170 | 9.227 | 5.909 | 25.591 | 1.00 | 0.92 | 1SG | 1379 |
| ATOM | 1379 | CG | LEU | 170 | 9.542 | 4.512 | 25.033 | 1.00 | 0.92 | 1SG | 1380 |
| ATOM | 1380 | CD2 | LEU | 170 | 8.617 | 4.147 | 23.880 | 1.00 | 0.92 | 1SG | 1381 |
| ATOM | 1381 | CD1 | LEU | 170 | 9.511 | 3.454 | 26.139 | 1.00 | 0.92 | 1SG | 1382 |
| ATOM | 1382 | C | LEU | 170 | 11.607 | 6.233 | 26.225 | 1.00 | 0.92 | 1SG | 1383 |
| ATOM | 1383 | O | LEU | 170 | 12.066 | 5.168 | 25.813 | 1.00 | 0.92 | 1SG | 1384 |
| ATOM | 1384 | N | LYS | 171 | 12.310 | 7.372 | 26.173 | 1.00 | 0.90 | 1SG | 1385 |
| ATOM | 1385 | CA | LYS | 171 | 13.685 | 7.441 | 25.767 | 1.00 | 0.90 | 1SG | 1386 |
| ATOM | 1386 | CB | LYS | 171 | 14.324 | 8.788 | 26.132 | 1.00 | 0.90 | 1SG | 1387 |
| ATOM | 1387 | CG | LYS | 171 | 14.438 | 9.029 | 27.637 | 1.00 | 0.90 | 1SG | 1388 |
| ATOM | 1388 | CD | LYS | 171 | 15.198 | 10.310 | 27.978 | 1.00 | 0.90 | 1SG | 1389 |
| ATOM | 1389 | CE | LYS | 171 | 16.583 | 10.387 | 27.337 | 1.00 | 0.90 | 1SG | 1390 |
| ATOM | 1390 | NZ | LYS | 171 | 17.148 | 11.739 | 27.533 | 1.00 | 0.90 | 1SG | 1391 |
| ATOM | 1391 | C | LYS | 171 | 14.520 | 6.362 | 26.400 | 1.00 | 0.90 | 1SG | 1392 |
| ATOM | 1392 | O | LYS | 171 | 14.079 | 5.606 | 27.264 | 1.00 | 0.90 | 1SG | 1393 |
| ATOM | 1393 | N | SER | 172 | 15.782 | 6.259 | 25.931 | 1.00 | 0.67 | 1SG | 1394 |
| ATOM | 1394 | CA | SER | 172 | 16.696 | 5.257 | 26.400 | 1.00 | 0.67 | 1SG | 1395 |
| ATOM | 1395 | CB | SER | 172 | 16.918 | 4.144 | 25.366 | 1.00 | 0.67 | 1SG | 1396 |
| ATOM | 1396 | OG | SER | 172 | 17.469 | 4.697 | 24.180 | 1.00 | 0.67 | 1SG | 1397 |
| ATOM | 1397 | C | SER | 172 | 18.038 | 5.890 | 26.605 | 1.00 | 0.67 | 1SG | 1398 |
| ATOM | 1398 | O | SER | 172 | 18.460 | 6.747 | 25.831 | 1.00 | 0.67 | 1SG | 1399 |
| ATOM | 1399 | N | SER | 173 | 18.745 | 5.498 | 27.684 | 1.00 | 0.73 | 1SG | 1400 |
| ATOM | 1400 | CA | SER | 173 | 20.077 | 5.997 | 27.872 | 1.00 | 0.73 | 1SG | 1401 |
| ATOM | 1401 | CB | SER | 173 | 20.686 | 5.631 | 29.236 | 1.00 | 0.73 | 1SG | 1402 |
| ATOM | 1402 | OG | SER | 173 | 21.999 | 6.162 | 29.344 | 1.00 | 0.73 | 1SG | 1403 |
| ATOM | 1403 | C | SER | 173 | 20.928 | 5.329 | 26.848 | 1.00 | 0.73 | 1SG | 1404 |
| ATOM | 1404 | O | SER | 173 | 21.692 | 5.969 | 26.129 | 1.00 | 0.73 | 1SG | 1405 |
| ATOM | 1405 | N | ILE | 174 | 20.773 | 3.998 | 26.759 | 1.00 | 0.88 | 1SG | 1406 |
| ATOM | 1406 | CA | ILE | 174 | 21.529 | 3.189 | 25.855 | 1.00 | 0.88 | 1SG | 1407 |
| ATOM | 1407 | CB | ILE | 174 | 22.323 | 2.115 | 26.563 | 1.00 | 0.88 | 1SG | 1408 |
| ATOM | 1408 | CG2 | ILE | 174 | 23.429 | 2.831 | 27.356 | 1.00 | 0.88 | 1SG | 1409 |
| ATOM | 1409 | CG1 | ILE | 174 | 21.449 | 1.195 | 27.443 | 1.00 | 0.88 | 1SG | 1410 |
| ATOM | 1410 | CD1 | ILE | 174 | 20.685 | 0.093 | 26.716 | 1.00 | 0.88 | 1SG | 1411 |
| ATOM | 1411 | C | ILE | 174 | 20.582 | 2.621 | 24.851 | 1.00 | 0.88 | 1SG | 1412 |
| ATOM | 1412 | O | ILE | 174 | 19.548 | 3.224 | 24.570 | 1.00 | 0.88 | 1SG | 1413 |
| ATOM | 1413 | N | GLN | 175 | 20.922 | 1.458 | 24.267 | 1.00 | 0.80 | 1SG | 1414 |
| ATOM | 1414 | CA | GLN | 175 | 20.121 | 0.866 | 23.237 | 1.00 | 0.80 | 1SG | 1415 |
| ATOM | 1415 | CB | GLN | 175 | 20.597 | −0.547 | 22.857 | 1.00 | 0.80 | 1SG | 1416 |
| ATOM | 1416 | CG | GLN | 175 | 20.537 | −1.545 | 24.015 | 1.00 | 0.80 | 1SG | 1417 |
| ATOM | 1417 | CD | GLN | 175 | 21.036 | −2.889 | 23.504 | 1.00 | 0.80 | 1SG | 1418 |
| ATOM | 1418 | OE1 | GLN | 175 | 21.026 | −3.150 | 22.302 | 1.00 | 0.80 | 1SG | 1419 |
| ATOM | 1419 | NE2 | GLN | 175 | 21.483 | −3.768 | 24.441 | 1.00 | 0.80 | 1SG | 1420 |
| ATOM | 1420 | C | GLN | 175 | 18.704 | 0.766 | 23.709 | 1.00 | 0.80 | 1SG | 1421 |
| ATOM | 1421 | O | GLN | 175 | 17.777 | 1.094 | 22.970 | 1.00 | 0.80 | 1SG | 1422 |
| ATOM | 1422 | N | TRP | 176 | 18.488 | 0.328 | 24.963 | 1.00 | 0.79 | 1SG | 1423 |
| ATOM | 1423 | CA | TRP | 176 | 17.144 | 0.214 | 25.444 | 1.00 | 0.79 | 1SG | 1424 |
| ATOM | 1424 | CB | TRP | 176 | 16.886 | −1.087 | 26.225 | 1.00 | 0.79 | 1SG | 1425 |
| ATOM | 1425 | CG | TRP | 176 | 16.925 | −2.329 | 25.364 | 1.00 | 0.79 | 1SG | 1426 |
| ATOM | 1426 | CD2 | TRP | 176 | 15.760 | −2.985 | 24.838 | 1.00 | 0.79 | 1SG | 1427 |
| ATOM | 1427 | CD1 | TRP | 176 | 18.001 | −3.050 | 24.932 | 1.00 | 0.79 | 1SG | 1428 |
| ATOM | 1428 | NE1 | TRP | 176 | 17.579 | −4.110 | 24.165 | 1.00 | 0.79 | 1SG | 1429 |
| ATOM | 1429 | CE2 | TRP | 176 | 16.202 | −4.083 | 24.100 | 1.00 | 0.79 | 1SG | 1430 |
| ATOM | 1430 | CE3 | TRP | 176 | 14.431 | −2.696 | 24.961 | 1.00 | 0.79 | 1SG | 1431 |
| ATOM | 1431 | CZ2 | TRP | 176 | 15.317 | −4.912 | 23.471 | 1.00 | 0.79 | 1SG | 1432 |
| ATOM | 1432 | CZ3 | TRP | 176 | 13.542 | −3.535 | 24.325 | 1.00 | 0.79 | 1SG | 1433 |
| ATOM | 1433 | CH2 | TRP | 176 | 13.977 | −4.621 | 23.594 | 1.00 | 0.79 | 1SG | 1434 |
| ATOM | 1434 | C | TRP | 176 | 16.873 | 1.367 | 26.358 | 1.00 | 0.79 | 1SG | 1435 |
| ATOM | 1435 | O | TRP | 176 | 17.787 | 1.941 | 26.948 | 1.00 | 0.79 | 1SG | 1436 |
| ATOM | 1436 | N | LYS | 177 | 15.583 | 1.744 | 26.468 | 1.00 | 0.95 | 1SG | 1437 |
| ATOM | 1437 | CA | LYS | 177 | 15.153 | 2.820 | 27.309 | 1.00 | 0.95 | 1SG | 1438 |
| ATOM | 1438 | CB | LYS | 177 | 13.625 | 2.901 | 27.429 | 1.00 | 0.95 | 1SG | 1439 |
| ATOM | 1439 | CG | LYS | 177 | 12.876 | 2.570 | 26.137 | 1.00 | 0.95 | 1SG | 1440 |
| ATOM | 1440 | CD | LYS | 177 | 12.910 | 1.075 | 25.804 | 1.00 | 0.95 | 1SG | 1441 |
| ATOM | 1441 | CE | LYS | 177 | 11.915 | 0.653 | 24.722 | 1.00 | 0.95 | 1SG | 1442 |
| ATOM | 1442 | NZ | LYS | 177 | 11.967 | −0.814 | 24.532 | 1.00 | 0.95 | 1SG | 1443 |
| ATOM | 1443 | C | LYS | 177 | 15.635 | 2.510 | 28.689 | 1.00 | 0.95 | 1SG | 1444 |
| ATOM | 1444 | O | LYS | 177 | 15.163 | 1.559 | 29.309 | 1.00 | 0.95 | 1SG | 1445 |
| ATOM | 1445 | N | TYR | 178 | 16.606 | 3.285 | 29.208 | 1.00 | 0.83 | 1SG | 1446 |
| ATOM | 1446 | CA | TYR | 178 | 17.047 | 3.013 | 30.546 | 1.00 | 0.83 | 1SG | 1447 |
| ATOM | 1447 | CB | TYR | 178 | 18.340 | 3.751 | 30.954 | 1.00 | 0.83 | 1SG | 1448 |
| ATOM | 1448 | CG | TYR | 178 | 18.095 | 5.173 | 31.334 | 1.00 | 0.83 | 1SG | 1449 |
| ATOM | 1449 | CD1 | TYR | 178 | 18.037 | 6.171 | 30.389 | 1.00 | 0.83 | 1SG | 1450 |
| ATOM | 1450 | CD2 | TYR | 178 | 17.922 | 5.507 | 32.658 | 1.00 | 0.83 | 1SG | 1451 |
| ATOM | 1451 | CE1 | TYR | 178 | 17.825 | 7.478 | 30.761 | 1..00 | 0.83 | 1SG | 1452 |
| ATOM | 1452 | CE2 | TYR | 178 | 17.711 | 6.811 | 33.037 | 1.00 | 0.83 | 1SG | 1453 |
| ATOM | 1453 | CZ | TYR | 178 | 17.668 | 7.801 | 32.086 | 1.00 | 0.83 | 1SG | 1454 |
| ATOM | 1454 | OH | TYR | 178 | 17.452 | 9.142 | 32.468 | 1.00 | 0.83 | 1SG | 1455 |
| ATOM | 1455 | C | TYR | 178 | 15.936 | 3.415 | 31.459 | 1.00 | 0.83 | 1SG | 1456 |
| ATOM | 1456 | O | TYR | 178 | 15.693 | 2.789 | 32.489 | 1.00 | 0.83 | 1SG | 1457 |
| ATOM | 1457 | N | VAL | 179 | 15.232 | 4.495 | 31.079 | 1.00 | 0.55 | 1SG | 1458 |
| ATOM | 1458 | CA | VAL | 179 | 14.127 | 5.034 | 31.816 | 1.00 | 0.55 | 1SG | 1459 |
| ATOM | 1459 | CB | VAL | 179 | 13.725 | 6.402 | 31.375 | 1.00 | 0.55 | 1SG | 1460 |
| ATOM | 1460 | CG1 | VAL | 179 | 14.761 | 7.424 | 31.862 | 1.00 | 0.55 | 1SG | 1461 |
| ATOM | 1461 | CG2 | VAL | 179 | 13.661 | 6.357 | 29.854 | 1.00 | 0.55 | 1SG | 1462 |
| ATOM | 1462 | C | VAL | 179 | 12.940 | 4.125 | 31.746 | 1.00 | 0.55 | 1SG | 1463 |
| ATOM | 1463 | O | VAL | 179 | 12.015 | 4.262 | 32.543 | 1.00 | 0.55 | 1SG | 1464 |
| ATOM | 1464 | N | ILE | 180 | 12.879 | 3.214 | 30.759 | 1.00 | 0.36 | 1SG | 1465 |
| ATOM | 1465 | CA | ILE | 180 | 11.735 | 2.346 | 30.712 | 1.00 | 0.36 | 1SG | 1466 |
| ATOM | 1466 | CB | ILE | 180 | 11.151 | 2.191 | 29.338 | 1.00 | 0.36 | 1SG | 1467 |
| ATOM | 1467 | CG2 | ILE | 180 | 10.143 | 1.029 | 29.347 | 1.00 | 0.36 | 1SG | 1468 |
| ATOM | 1468 | CG1 | ILE | 180 | 10.544 | 3.528 | 28.894 | 1.00 | 0.36 | 1SG | 1469 |
| ATOM | 1469 | CD1 | ILE | 180 | 11.581 | 4.626 | 28.692 | 1.00 | 0.36 | 1SG | 1470 |
| ATOM | 1470 | C | ILE | 180 | 12.140 | 0.996 | 31.204 | 1.00 | 0.36 | 1SG | 1471 |
| ATOM | 1471 | O | ILE | 180 | 13.280 | 0.568 | 31.029 | 1.00 | 0.36 | 1SG | 1472 |
| ATOM | 1472 | N | ASN | 181 | 11.192 | 0.302 | 31.865 | 1.00 | 0.37 | 1SG | 1473 |
| ATOM | 1473 | CA | ASN | 181 | 11.429 | −0.998 | 32.413 | 1.00 | 0.37 | 1SG | 1474 |
| ATOM | 1474 | CB | ASN | 181 | 10.672 | −1.260 | 33.729 | 1.00 | 0.37 | 1SG | 1475 |
| ATOM | 1475 | CG | ASN | 181 | 11.194 | −2.556 | 34.338 | 1.00 | 0.37 | 1SG | 1476 |
| ATOM | 1476 | OD1 | ASN | 181 | 11.853 | −3.352 | 33.672 | 1.00 | 0.37 | 1SG | 1477 |
| ATOM | 1477 | ND2 | ASN | 181 | 10.887 | −2.781 | 35.644 | 1.00 | 0.37 | 1SG | 1478 |
| ATOM | 1478 | C | ASN | 181 | 10.963 | −2.003 | 31.412 | 1.00 | 0.37 | 1SG | 1479 |
| ATOM | 1479 | O | ASN | 181 | 9.804 | −2.010 | 30.999 | 1.00 | 0.37 | 1SG | 1480 |
| ATOM | 1480 | N | LEU | 182 | 11.890 | −2.886 | 31.001 | 1.00 | 0.50 | 1SG | 1481 |
| ATOM | 1481 | CA | LEU | 182 | 11.646 | −3.916 | 30.035 | 1.00 | 0.50 | 1SG | 1482 |
| ATOM | 1482 | CB | LEU | 182 | 12.931 | −4.707 | 29.709 | 1.00 | 0.50 | 1SG | 1483 |
| ATOM | 1483 | CG | LEU | 182 | 12.793 | −5.841 | 28.668 | 1.00 | 0.50 | 1SG | 1484 |
| ATOM | 1484 | CD2 | LEU | 182 | 12.227 | −5.306 | 27.344 | 1.00 | 0.50 | 1SG | 1485 |
| ATOM | 1485 | CD1 | LEU | 182 | 12.009 | −7.051 | 29.208 | 1.00 | 0.50 | 1SG | 1486 |
| ATOM | 1486 | C | LEU | 182 | 10.644 | −4.860 | 30.615 | 1.00 | 0.50 | 1SG | 1487 |
| ATOM | 1487 | O | LEU | 182 | 9.826 | −5.431 | 29.895 | 1.00 | 0.50 | 1SG | 1488 |
| ATOM | 1488 | N | CYS | 183 | 10.694 | −5.038 | 31.946 | 1.00 | 0.40 | 1SG | 1489 |
| ATOM | 1489 | CA | CYS | 183 | 9.862 | −5.980 | 32.631 | 1.00 | 0.40 | 1SG | 1490 |
| ATOM | 1490 | CB | CYS | 183 | 10.109 | −5.994 | 34.150 | 1.00 | 0.40 | 1SG | 1491 |
| ATOM | 1491 | SG | CYS | 183 | 11.781 | −6.557 | 34.585 | 1.00 | 0.40 | 1SG | 1492 |
| ATOM | 1492 | C | CYS | 183 | 8.418 | −5.651 | 32.425 | 1.00 | 0.40 | 1SG | 1493 |
| ATOM | 1493 | O | CYS | 183 | 7.605 | −6.558 | 32.255 | 1.00 | 0.40 | 1SG | 1494 |
| ATOM | 1494 | N | GLY | 184 | 8.049 | −4.354 | 32.431 | 1.00 | 0.35 | 1SG | 1495 |
| ATOM | 1495 | CA | GLY | 184 | 6.652 | −4.030 | 32.329 | 1.00 | 0.35 | 1SG | 1496 |
| ATOM | 1496 | C | GLY | 184 | 6.319 | −3.544 | 30.953 | 1.00 | 0.35 | 1SG | 1497 |
| ATOM | 1497 | O | GLY | 184 | 6.058 | −2.362 | 30.738 | 1.00 | 0.35 | 1SG | 1498 |
| ATOM | 1498 | N | GLN | 185 | 6.289 | −4.471 | 29.985 | 1.00 | 0.51 | 1SG | 1499 |
| ATOM | 1499 | CA | GLN | 185 | 5.963 | −4.188 | 28.618 | 1.00 | 0.51 | 1SG | 1500 |
| ATOM | 1500 | CB | GLN | 185 | 6.015 | −5.470 | 27.767 | 1.00 | 0.51 | 1SG | 1501 |
| ATOM | 1501 | CG | GLN | 185 | 7.331 | −6.252 | 27.848 | 1.00 | 0.51 | 1SG | 1502 |
| ATOM | 1502 | CD | GLN | 185 | 7.199 | −7.365 | 28.881 | 1.00 | 0.51 | 1SG | 1503 |
| ATOM | 1503 | OE1 | GLN | 185 | 7.923 | −8.358 | 28.811 | 1.00 | 0.51 | 1SG | 1504 |
| ATOM | 1504 | NE2 | GLN | 185 | 6.259 | −7.211 | 29.851 | 1.00 | 0.51 | 1SG | 1505 |
| ATOM | 1505 | C | GLN | 185 | 4.530 | −3.755 | 28.578 | 1.00 | 0.51 | 1SG | 1506 |
| ATOM | 1506 | O | GLN | 185 | 4.143 | −2.870 | 27.814 | 1.00 | 0.51 | 1SG | 1507 |
| ATOM | 1507 | N | ASP | 186 | 3.706 | −4.439 | 29.390 | 1.00 | 0.60 | 1SG | 1508 |
| ATOM | 1508 | CA | ASP | 186 | 2.281 | −4.280 | 29.456 | 1.00 | 0.60 | 1SG | 1509 |
| ATOM | 1509 | CB | ASP | 186 | 1.589 | −5.455 | 30.171 | 1.00 | 0.60 | 1SG | 1510 |
| ATOM | 1510 | CG | ASP | 186 | 2.080 | −5.530 | 31.609 | 1.00 | 0.60 | 1SG | 1511 |
| ATOM | 1511 | OD1 | ASP | 186 | 3.166 | −4.960 | 31.903 | 1.00 | 0.60 | 1SG | 1512 |
| ATOM | 1512 | OD2 | ASP | 186 | 1.374 | −6.169 | 32.434 | 1.00 | 0.60 | 1SG | 1513 |
| ATOM | 1513 | C | ASP | 186 | 1.826 | −3.014 | 30.128 | 1.00 | 0.60 | 1SG | 1514 |
| ATOM | 1514 | O | ASP | 186 | 0.811 | −2.453 | 29.718 | 1.00 | 0.60 | 1SG | 1515 |
| ATOM | 1515 | N | PHE | 187 | 2.539 | −2.524 | 31.164 | 1.00 | 0.51 | 1SG | 1516 |
| ATOM | 1516 | CA | PHE | 187 | 2.016 | −1.444 | 31.966 | 1.00 | 0.51 | 1SG | 1517 |
| ATOM | 1517 | CB | PHE | 187 | 2.923 | −1.000 | 33.125 | 1.00 | 0.51 | 1SG | 1518 |
| ATOM | 1518 | CG | PHE | 187 | 2.838 | −2.044 | 34.183 | 1.00 | 0.51 | 1SG | 1519 |
| ATOM | 1519 | CD1 | PHE | 187 | 1.746 | −2.088 | 35.021 | 1.00 | 0.51 | 1SG | 1520 |
| ATOM | 1520 | CD2 | PHE | 187 | 3.845 | −2.967 | 34.351 | 1.00 | 0.51 | 1SG | 1521 |
| ATOM | 1521 | CE1 | PHE | 187 | 1.651 | −3.043 | 36.005 | 1.00 | 0.51 | 1SG | 1522 |
| ATOM | 1522 | CE2 | PHE | 187 | 3.754 | −3.925 | 35.335 | 1.00 | 0.51 | 1SG | 1523 |
| ATOM | 1523 | CZ | PHE | 187 | 2.657 | −3.966 | 36.162 | 1.00 | 0.51 | 1SG | 1524 |
| ATOM | 1524 | C | PHE | 187 | 1.618 | −0.229 | 31.182 | 1.00 | 0.51 | 1SG | 1525 |
| ATOM | 1525 | O | PHE | 187 | 2.370 | 0.341 | 30.393 | 1.00 | 0.51 | 1SG | 1526 |
| ATOM | 1526 | N | PRO | 188 | 0.370 | 0.117 | 31.416 | 1.00 | 0.41 | 1SG | 1527 |
| ATOM | 1527 | CA | PRO | 188 | −0.247 | 1.287 | 30.832 | 1.00 | 0.41 | 1SG | 1528 |
| ATOM | 1528 | CD | PRO | 188 | −0.605 | −0.941 | 31.634 | 1.00 | 0.41 | 1SG | 1529 |
| ATOM | 1529 | CB | PRO | 188 | −1.671 | 0.873 | 30.465 | 1.00 | 0.41 | 1SG | 1530 |
| ATOM | 1530 | CG | PRO | 188 | −1.977 | −0.289 | 31.420 | 1.00 | 0.41 | 1SG | 1531 |
| ATOM | 1531 | C | PRO | 188 | −0.261 | 2.440 | 31.799 | 1.00 | 0.41 | 1SG | 1532 |
| ATOM | 1532 | O | PRO | 188 | 0.053 | 2.242 | 32.971 | 1.00 | 0.41 | 1SG | 1533 |
| ATOM | 1533 | N | LEU | 189 | −0.673 | 3.644 | 31.340 | 1.00 | 0.54 | 1SG | 1534 |
| ATOM | 1534 | CA | LEU | 189 | −0.695 | 4.815 | 32.180 | 1.00 | 0.54 | 1SG | 1535 |
| ATOM | 1535 | CB | LEU | 189 | 0.429 | 5.803 | 31.864 | 1.00 | 0.54 | 1SG | 1536 |
| ATOM | 1536 | CG | LEU | 189 | 1.873 | 5.305 | 31.913 | 1.00 | 0.54 | 1SG | 1537 |
| ATOM | 1537 | CD2 | LEU | 189 | 2.768 | 6.464 | 31.455 | 1.00 | 0.54 | 1SG | 1538 |
| ATOM | 1538 | CD1 | LEU | 189 | 2.102 | 4.063 | 31.034 | 1.00 | 0.54 | 1SG | 1539 |
| ATOM | 1539 | C | LEU | 189 | −1.854 | 5.662 | 31.747 | 1.00 | 0.54 | 1SG | 1540 |
| ATOM | 1540 | O | LEU | 189 | −2.424 | 5.462 | 30.676 | 1.00 | 0.54 | 1SG | 1541 |
| ATOM | 1541 | N | LYS | 190 | −2.206 | 6.658 | 32.590 | 1.00 | 0.65 | 1SG | 1542 |
| ATOM | 1542 | CA | LYS | 190 | −3.135 | 7.683 | 32.204 | 1.00 | 0.65 | 1SG | 1543 |
| ATOM | 1543 | CB | LYS | 190 | −4.244 | 8.007 | 33.224 | 1.00 | 0.65 | 1SG | 1544 |
| ATOM | 1544 | CG | LYS | 190 | −3.719 | 8.638 | 34.517 | 1.00 | 0.65 | 1SG | 1545 |
| ATOM | 1545 | CD | LYS | 190 | −4.772 | 9.431 | 35.300 | 1.00 | 0.65 | 1SG | 1546 |
| ATOM | 1546 | CE | LYS | 190 | −5.979 | 8.609 | 35.755 | 1.00 | 0.65 | 1SG | 1547 |
| ATOM | 1547 | NZ | LYS | 190 | −6.928 | 9.476 | 36.491 | 1.00 | 0.65 | 1SG | 1548 |
| ATOM | 1548 | C | LYS | 190 | −2.277 | 8.903 | 32.146 | 1.00 | 0.65 | 1SG | 1549 |
| ATOM | 1549 | O | LYS | 190 | −1.268 | 8.958 | 32.843 | 1.00 | 0.65 | 1SG | 1550 |
| ATOM | 1550 | N | SER | 191 | −2.590 | 9.894 | 31.288 | 1.00 | 0.47 | 1SG | 1551 |
| ATOM | 1551 | CA | SER | 191 | −1.761 | 11.072 | 31.284 | 1.00 | 0.47 | 1SG | 1552 |
| ATOM | 1552 | CB | SER | 191 | −0.682 | 11.069 | 30.192 | 1.00 | 0.47 | 1SG | 1553 |
| ATOM | 1553 | OG | SER | 191 | 0.083 | 12.263 | 30.258 | 1.00 | 0.47 | 1SG | 1554 |
| ATOM | 1554 | C | SER | 191 | −2.623 | 12.275 | 31.055 | 1.00 | 0.47 | 1SG | 1555 |
| ATOM | 1555 | O | SER | 191 | −3.482 | 12.281 | 30.175 | 1.00 | 0.47 | 1SG | 1556 |
| ATOM | 1556 | N | ASN | 192 | −2.402 | 13.328 | 31.868 | 1.00 | 0.28 | 1SG | 1557 |
| ATOM | 1557 | CA | ASN | 192 | −3.095 | 14.582 | 31.771 | 1.00 | 0.28 | 1SG | 1558 |
| ATOM | 1558 | CB | ASN | 192 | −2.873 | 15.509 | 32.980 | 1.00 | 0.28 | 1SG | 1559 |
| ATOM | 1559 | CG | ASN | 192 | −3.558 | 14.872 | 34.173 | 1.00 | 0.28 | 1SG | 1560 |
| ATOM | 1560 | OD1 | ASN | 192 | −2.985 | 13.977 | 34.789 | 1.00 | 0.28 | 1SG | 1561 |
| ATOM | 1561 | ND2 | ASN | 192 | −4.792 | 15.334 | 34.511 | 1.00 | 0.28 | 1SG | 1562 |
| ATOM | 1562 | C | ASN | 192 | −2.608 | 15.276 | 30.553 | 1.00 | 0.28 | 1SG | 1563 |
| ATOM | 1563 | O | ASN | 192 | −3.303 | 16.128 | 30.001 | 1.00 | 0.28 | 1SG | 1564 |
| ATOM | 1564 | N | PHE | 193 | −1.356 | 14.977 | 30.152 | 1.00 | 0.34 | 1SG | 1565 |
| ATOM | 1565 | CA | PHE | 193 | −0.838 | 15.559 | 28.955 | 1.00 | 0.34 | 1SG | 1566 |
| ATOM | 1566 | CB | PHE | 193 | 0.657 | 15.257 | 28.754 | 1.00 | 0.34 | 1SG | 1567 |
| ATOM | 1567 | CG | PHE | 193 | 1.232 | 16.278 | 27.836 | 1.00 | 0.34 | 1SG | 1568 |
| ATOM | 1568 | CD1 | PHE | 193 | 1.624 | 17.496 | 28.340 | 1.00 | 0.34 | 1SG | 1569 |
| ATOM | 1569 | CD2 | PHE | 193 | 1.396 | 16.031 | 26.495 | 1.00 | 0.34 | 1SG | 1570 |
| ATOM | 1570 | CE1 | PHE | 193 | 2.162 | 18.463 | 27.527 | 1.00 | 0.34 | 1SG | 1571 |
| ATOM | 1571 | CE2 | PHE | 193 | 1.935 | 16.992 | 25.673 | 1.00 | 0.34 | 1SG | 1572 |
| ATOM | 1572 | CZ | PHE | 193 | 2.317 | 18.209 | 26.187 | 1.00 | 0.34 | 1SG | 1573 |
| ATOM | 1573 | C | PHE | 193 | −1.626 | 14.826 | 27.932 | 1.00 | 0.34 | 1SG | 1574 |
| ATOM | 1574 | O | PHE | 193 | −1.232 | 13.756 | 27.471 | 1.00 | 0.34 | 1SG | 1575 |
| ATOM | 1575 | N | GLU | 194 | −2.768 | 15.426 | 27.559 | 1.00 | 0.57 | 1SG | 1576 |
| ATOM | 1576 | CA | GLU | 194 | −3.794 | 14.805 | 26.788 | 1.00 | 0.57 | 1SG | 1577 |
| ATOM | 1577 | CB | GLU | 194 | −4.884 | 15.812 | 26.375 | 1.00 | 0.57 | 1SG | 1578 |
| ATOM | 1578 | CG | GLU | 194 | −6.009 | 15.179 | 25.556 | 1.00 | 0.57 | 1SG | 1579 |
| ATOM | 1579 | CD | GLU | 194 | −7.032 | 16.248 | 25.187 | 1.00 | 0.57 | 1SG | 1580 |
| ATOM | 1580 | OE1 | GLU | 194 | −6.617 | 17.335 | 24.706 | 1.00 | 0.57 | 1SG | 1581 |
| ATOM | 1581 | OE2 | GLU | 194 | −8.251 | 15.980 | 25.373 | 1.00 | 0.57 | 1SG | 1582 |
| ATOM | 1582 | C | GLU | 194 | −3.211 | 14.249 | 25.548 | 1.00 | 0.57 | 1SG | 1583 |
| ATOM | 1583 | O | GLU | 194 | −3.430 | 13.085 | 25.213 | 1.00 | 0.57 | 1SG | 1584 |
| ATOM | 1584 | N | LEU | 195 | −2.429 | 15.062 | 24.833 | 1.00 | 0.64 | 1SG | 1585 |
| ATOM | 1585 | CA | LEU | 195 | −1.973 | 14.518 | 23.602 | 1.00 | 0.64 | 1SG | 1586 |
| ATOM | 1586 | CB | LEU | 195 | −2.324 | 15.379 | 22.378 | 1.00 | 0.64 | 1SG | 1587 |
| ATOM | 1587 | CG | LEU | 195 | −3.835 | 15.584 | 22.176 | 1.00 | 0.64 | 1SG | 1588 |
| ATOM | 1588 | CD2 | LEU | 195 | −4.136 | 16.242 | 20.824 | 1.00 | 0.64 | 1SG | 1589 |
| ATOM | 1589 | CD1 | LEU | 195 | −4.443 | 16.368 | 23.348 | 1.00 | 0.64 | 1SG | 1590 |
| ATOM | 1590 | C | LEU | 195 | −0.501 | 14.482 | 23.658 | 1.00 | 0.64 | 1SG | 1591 |
| ATOM | 1591 | O | LEU | 195 | 0.171 | 15.415 | 23.223 | 1.00 | 0.64 | 1SG | 1592 |
| ATOM | 1592 | N | VAL | 196 | 0.067 | 13.410 | 24.215 | 1.00 | 0.58 | 1SG | 1593 |
| ATOM | 1593 | CA | VAL | 196 | 1.482 | 13.473 | 24.173 | 1.00 | 0.58 | 1SG | 1594 |
| ATOM | 1594 | CB | VAL | 196 | 2.158 | 12.479 | 25.057 | 1.00 | 0.58 | 1SG | 1595 |
| ATOM | 1595 | CG1 | VAL | 196 | 3.603 | 12.962 | 25.179 | 1.00 | 0.58 | 1SG | 1596 |
| ATOM | 1596 | CG2 | VAL | 196 | 1.427 | 12.403 | 26.409 | 1.00 | 0.58 | 1SG | 1597 |
| ATOM | 1597 | C | VAL | 196 | 1.810 | 13.262 | 22.735 | 1.00 | 0.58 | 1SG | 1598 |
| ATOM | 1598 | O | VAL | 196 | 2.743 | 13.836 | 22.177 | 1.00 | 0.58 | 1SG | 1599 |
| ATOM | 1599 | N | SER | 197 | 1.001 | 12.405 | 22.096 | 1.00 | 0.70 | 1SG | 1600 |
| ATOM | 1600 | CA | SER | 197 | 1.119 | 12.119 | 20.702 | 1.00 | 0.70 | 1SG | 1601 |
| ATOM | 1601 | CB | SER | 197 | 2.137 | 10.997 | 20.471 | 1.00 | 0.70 | 1SG | 1602 |
| ATOM | 1602 | OG | SER | 197 | 3.417 | 11.415 | 20.913 | 1.00 | 0.70 | 1SG | 1603 |
| ATOM | 1603 | C | SER | 197 | −0.254 | 11.684 | 20.335 | 1.00 | 0.70 | 1SG | 1604 |
| ATOM | 1604 | O | SER | 197 | −0.611 | 10.512 | 20.414 | 1.00 | 0.70 | 1SG | 1605 |
| ATOM | 1605 | N | GLU | 198 | −1.049 | 12.676 | 19.920 | 1.00 | 0.80 | 1SG | 1606 |
| ATOM | 1606 | CA | GLU | 198 | −2.454 | 12.572 | 19.714 | 1.00 | 0.80 | 1SG | 1607 |
| ATOM | 1607 | CB | GLU | 198 | −3.034 | 13.946 | 19.355 | 1.00 | 0.80 | 1SG | 1608 |
| ATOM | 1608 | CG | GLU | 198 | −2.301 | 14.605 | 18.187 | 1.00 | 0.80 | 1SG | 1609 |
| ATOM | 1609 | CD | GLU | 198 | −2.980 | 15.934 | 17.902 | 1.00 | 0.80 | 1SG | 1610 |
| ATOM | 1610 | OE1 | GLU | 198 | −4.222 | 16.022 | 18.089 | 1.00 | 0.80 | 1SG | 1611 |
| ATOM | 1611 | OE2 | GLU | 198 | −2.261 | 16.876 | 17.477 | 1.00 | 0.80 | 1SG | 1612 |
| ATOM | 1612 | C | GLU | 198 | −2.891 | 11.631 | 18.637 | 1.00 | 0.80 | 1SG | 1613 |
| ATOM | 1613 | O | GLU | 198 | −3.615 | 10.674 | 18.910 | 1.00 | 0.80 | 1SG | 1614 |
| ATOM | 1614 | N | LEU | 199 | −2.460 | 11.875 | 17.384 | 1.00 | 0.97 | 1SG | 1615 |
| ATOM | 1615 | CA | LEU | 199 | −3.132 | 11.224 | 16.288 | 1.00 | 0.97 | 1SG | 1616 |
| ATOM | 1616 | CB | LEU | 199 | −3.150 | 12.122 | 15.038 | 1.00 | 0.97 | 1SG | 1617 |
| ATOM | 1617 | CG | LEU | 199 | −3.916 | 13.410 | 15.345 | 1.00 | 0.97 | 1SG | 1618 |
| ATOM | 1618 | CD2 | LEU | 199 | −5.232 | 13.014 | 16.027 | 1.00 | 0.97 | 1SG | 1619 |
| ATOM | 1619 | CD1 | LEU | 199 | −4.047 | 14.372 | 14.144 | 1.00 | 0.97 | 1SG | 1620 |
| ATOM | 1620 | C | LEU | 199 | −2.785 | 9.791 | 15.944 | 1.00 | 0.97 | 1SG | 1621 |
| ATOM | 1621 | O | LEU | 199 | −3.698 | 8.971 | 15.909 | 1.00 | 0.97 | 1SG | 1622 |
| ATOM | 1622 | N | LYS | 200 | −1.513 | 9.443 | 15.577 | 1.00 | 1.34 | 1SG | 1623 |
| ATOM | 1623 | CA | LYS | 200 | −1.236 | 8.068 | 15.193 | 1.00 | 1.34 | 1SG | 1624 |
| ATOM | 1624 | CB | LYS | 200 | −2.218 | 7.404 | 14.223 | 1.00 | 1.34 | 1SG | 1625 |
| ATOM | 1625 | CG | LYS | 200 | −1.656 | 6.962 | 12.871 | 1.00 | 1.34 | 1SG | 1626 |
| ATOM | 1626 | CD | LYS | 200 | −1.021 | 5.572 | 12.852 | 1.00 | 1.34 | 1SG | 1627 |
| ATOM | 1627 | CE | LYS | 200 | −0.673 | 5.075 | 11.448 | 1.00 | 1.34 | 1SG | 1628 |
| ATOM | 1628 | NZ | LYS | 200 | −0.449 | 3.614 | 11.497 | 1.00 | 1.34 | 1SG | 1629 |
| ATOM | 1629 | C | LYS | 200 | 0.140 | 7.918 | 14.636 | 1.00 | 1.34 | 1SG | 1630 |
| ATOM | 1630 | O | LYS | 200 | 0.680 | 6.818 | 14.577 | 1.00 | 1.34 | 1SG | 1631 |
| ATOM | 1631 | N | LYS | 201 | 0.779 | 9.022 | 14.266 | 1.00 | 1.35 | 1SG | 1632 |
| ATOM | 1632 | CA | LYS | 201 | 2.182 | 8.929 | 14.034 | 1.00 | 1.35 | 1SG | 1633 |
| ATOM | 1633 | CB | LYS | 201 | 2.681 | 9.414 | 12.664 | 1.00 | 1.35 | 1SG | 1634 |
| ATOM | 1634 | CG | LYS | 201 | 3.306 | 8.229 | 11.914 | 1.00 | 1.35 | 1SG | 1635 |
| ATOM | 1635 | CD | LYS | 201 | 2.354 | 7.048 | 11.724 | 1.00 | 1.35 | 1SG | 1636 |
| ATOM | 1636 | CE | LYS | 201 | 3.049 | 5.817 | 11.144 | 1.00 | 1.35 | 1SG | 1637 |
| ATOM | 1637 | NZ | LYS | 201 | 2.241 | 4.614 | 11.455 | 1.00 | 1.35 | 1SG | 1638 |
| ATOM | 1638 | C | LYS | 201 | 2.669 | 9.735 | 15.168 | 1.00 | 1.35 | 1SG | 1639 |
| ATOM | 1639 | O | LYS | 201 | 3.627 | 10.505 | 15.124 | 1.00 | 1.35 | 1SG | 1640 |
| ATOM | 1640 | N | LEU | 202 | 1.819 | 9.593 | 16.197 | 1.00 | 0.89 | 1SG | 1641 |
| ATOM | 1641 | CA | LEU | 202 | 1.872 | 10.023 | 17.544 | 1.00 | 0.89 | 1SG | 1642 |
| ATOM | 1642 | CB | LEU | 202 | 1.104 | 11.336 | 17.747 | 1.00 | 0.89 | 1SG | 1643 |
| ATOM | 1643 | CG | LEU | 202 | 1.804 | 12.529 | 17.071 | 1.00 | 0.89 | 1SG | 1644 |
| ATOM | 1644 | CD2 | LEU | 202 | 1.333 | 13.867 | 17.653 | 1.00 | 0.89 | 1SG | 1645 |
| ATOM | 1645 | CD1 | LEU | 202 | 1.675 | 12.475 | 15.544 | 1.00 | 0.89 | 1SG | 1646 |
| ATOM | 1646 | C | LEU | 202 | 1.123 | 8.895 | 18.182 | 1.00 | 0.89 | 1SG | 1647 |
| ATOM | 1647 | O | LEU | 202 | 0.953 | 8.800 | 19.392 | 1.00 | 0.89 | 1SG | 1648 |
| ATOM | 1648 | N | ASN | 203 | 0.799 | 7.917 | 17.315 | 1.00 | 0.45 | 1SG | 1649 |
| ATOM | 1649 | CA | ASN | 203 | −0.085 | 6.810 | 17.533 | 1.00 | 0.45 | 1SG | 1650 |
| ATOM | 1650 | CB | ASN | 203 | 0.502 | 5.568 | 18.204 | 1.00 | 0.45 | 1SG | 1651 |
| ATOM | 1651 | CG | ASN | 203 | 1.163 | 4.726 | 17.132 | 1.00 | 0.45 | 1SG | 1652 |
| ATOM | 1652 | OD1 | ASN | 203 | 0.739 | 4.714 | 15.978 | 1.00 | 0.45 | 1SG | 1653 |
| ATOM | 1653 | ND2 | ASN | 203 | 2.219 | 3.973 | 17.525 | 1.00 | 0.45 | 1SG | 1654 |
| ATOM | 1654 | C | ASN | 203 | −1.257 | 7.266 | 18.312 | 1.00 | 0.45 | 1SG | 1655 |
| ATOM | 1655 | O | ASN | 203 | −1.169 | 7.446 | 19.515 | 1.00 | 0.45 | 1SG | 1656 |
| ATOM | 1656 | N | GLY | 204 | −2.399 | 7.520 | 17.658 | 1.00 | 0.22 | 1SG | 1657 |
| ATOM | 1657 | CA | GLY | 204 | −3.492 | 7.977 | 18.447 | 1.00 | 0.22 | 1SG | 1658 |
| ATOM | 1658 | C | GLY | 204 | −4.754 | 7.366 | 17.942 | 1.00 | 0.22 | 1SG | 1659 |
| ATOM | 1659 | O | GLY | 204 | −4.850 | 6.901 | 16.803 | 1.00 | 0.22 | 1SG | 1660 |
| ATOM | 1660 | N | ALA | 205 | −5.756 | 7.352 | 18.841 | 1.00 | 0.24 | 1SG | 1661 |
| ATOM | 1661 | CA | ALA | 205 | −7.075 | 6.875 | 18.570 | 1.00 | 0.24 | 1SG | 1662 |
| ATOM | 1662 | CB | ALA | 205 | −7.450 | 5.617 | 19.367 | 1.00 | 0.24 | 1SG | 1663 |
| ATOM | 1663 | C | ALA | 205 | −7.978 | 7.960 | 19.040 | 1.00 | 0.24 | 1SG | 1664 |
| ATOM | 1664 | O | ALA | 205 | −7.880 | 8.413 | 20.179 | 1.00 | 0.24 | 1SG | 1665 |
| ATOM | 1665 | N | ASN | 206 | −8.892 | 8.398 | 18.161 | 1.00 | 0.46 | 1SG | 1666 |
| ATOM | 1666 | CA | ASN | 206 | −9.748 | 9.494 | 18.487 | 1.00 | 0.46 | 1SG | 1667 |
| ATOM | 1667 | CB | ASN | 206 | −9.531 | 10.672 | 17.514 | 1.00 | 0.46 | 1SG | 1668 |
| ATOM | 1668 | CG | ASN | 206 | −9.937 | 11.993 | 18.157 | 1.00 | 0.46 | 1SG | 1669 |
| ATOM | 1669 | OD1 | ASN | 206 | −9.620 | 13.061 | 17.637 | 1.00 | 0.46 | 1SG | 1670 |
| ATOM | 1670 | ND2 | ASN | 206 | −10.636 | 11.934 | 19.318 | 1.00 | 0.46 | 1SG | 1671 |
| ATOM | 1671 | C | ASN | 206 | −11.145 | 8.982 | 18.339 | 1.00 | 0.46 | 1SG | 1672 |
| ATOM | 1672 | O | ASN | 206 | −11.387 | 8.011 | 17.623 | 1.00 | 0.46 | 1SG | 1673 |
| ATOM | 1673 | N | MET | 207 | −12.097 | 9.633 | 19.028 | 1.00 | 0.60 | 1SG | 1674 |
| ATOM | 1674 | CA | MET | 207 | −13.462 | 9.209 | 19.044 | 1.00 | 0.60 | 1SG | 1675 |
| ATOM | 1675 | CB | MET | 207 | −14.121 | 9.435 | 20.411 | 1.00 | 0.60 | 1SG | 1676 |
| ATOM | 1676 | CG | MET | 207 | −15.588 | 9.018 | 20.470 | 1.00 | 0.60 | 1SG | 1677 |
| ATOM | 1677 | SD | MET | 207 | −16.370 | 9.334 | 22.079 | 1.00 | 0.60 | 1SG | 1678 |
| ATOM | 1678 | CE | MET | 207 | −16.155 | 11.136 | 22.007 | 1.00 | 0.60 | 1SG | 1679 |
| ATOM | 1679 | C | MET | 207 | −14.234 | 10.019 | 18.062 | 1.00 | 0.60 | 1SG | 1680 |
| ATOM | 1680 | O | MET | 207 | −13.927 | 11.180 | 17.803 | 1.00 | 0.60 | 1SG | 1681 |
| ATOM | 1681 | N | LEU | 208 | −15.283 | 9.393 | 17.503 | 1.00 | 0.62 | 1SG | 1682 |
| ATOM | 1682 | CA | LEU | 208 | −16.142 | 9.978 | 16.519 | 1.00 | 0.62 | 1SG | 1683 |
| ATOM | 1683 | CB | LEU | 208 | −17.313 | 9.007 | 16.215 | 1.00 | 0.62 | 1SG | 1684 |
| ATOM | 1684 | CG | LEU | 208 | −18.394 | 9.399 | 15.174 | 1.00 | 0.62 | 1SG | 1685 |
| ATOM | 1685 | CD2 | LEU | 208 | −19.088 | 10.737 | 15.466 | 1.00 | 0.62 | 1SG | 1686 |
| ATOM | 1686 | CD1 | LEU | 208 | −19.428 | 8.264 | 15.059 | 1.00 | 0.62 | 1SG | 1687 |
| ATOM | 1687 | C | LEU | 208 | −16.716 | 11.207 | 17.127 | 1.00 | 0.62 | 1SG | 1688 |
| ATOM | 1688 | O | LEU | 208 | −16.646 | 12.297 | 16.559 | 1.00 | 0.62 | 1SG | 1689 |
| ATOM | 1689 | N | GLU | 209 | −17.284 | 11.054 | 18.331 | 1.00 | 0.69 | 1SG | 1690 |
| ATOM | 1690 | CA | GLU | 209 | −17.981 | 12.157 | 18.902 | 1.00 | 0.69 | 1SG | 1691 |
| ATOM | 1691 | CB | GLU | 209 | −18.622 | 11.799 | 20.252 | 1.00 | 0.69 | 1SG | 1692 |
| ATOM | 1692 | CG | GLU | 209 | −19.747 | 12.748 | 20.652 | 1.00 | 0.69 | 1SG | 1693 |
| ATOM | 1693 | CD | GLU | 209 | −20.926 | 12.418 | 19.747 | 1.00 | 0.69 | 1SG | 1694 |
| ATOM | 1694 | OE1 | GLU | 209 | −20.748 | 11.562 | 18.840 | 1.00 | 0.69 | 1SG | 1695 |
| ATOM | 1695 | OE2 | GLU | 209 | −22.019 | 13.012 | 19.947 | 1.00 | 0.69 | 1SG | 1696 |
| ATOM | 1696 | C | GLU | 209 | −17.042 | 13.286 | 19.134 | 1.00 | 0.69 | 1SG | 1697 |
| ATOM | 1697 | O | GLU | 209 | −17.249 | 14.374 | 18.605 | 1.00 | 0.69 | 1SG | 1698 |
| ATOM | 1698 | N | THR | 210 | −15.952 | 13.036 | 19.882 | 1.00 | 0.73 | 1SG | 1699 |
| ATOM | 1699 | CA | THR | 210 | −15.086 | 14.104 | 20.294 | 1.00 | 0.73 | 1SG | 1700 |
| ATOM | 1700 | CB | TER | 210 | −14.095 | 13.701 | 21.349 | 1.00 | 0.73 | 1SG | 1701 |
| ATOM | 1701 | OG1 | THR | 210 | −13.422 | 14.850 | 21.843 | 1.00 | 0.73 | 1SG | 1702 |
| ATOM | 1702 | CG2 | THR | 210 | −13.083 | 12.711 | 20.754 | 1.00 | 0.73 | 1SG | 1703 |
| ATOM | 1703 | C | THR | 210 | −14.309 | 14.725 | 19.180 | 1.00 | 0.73 | 1SG | 1704 |
| ATOM | 1704 | O | THR | 210 | −14.243 | 15.951 | 19.095 | 1.00 | 0.73 | 1SG | 1705 |
| ATOM | 1705 | N | VAL | 211 | −13.719 | 13.910 | 18.284 | 1.00 | 0.59 | 1SG | 1706 |
| ATOM | 1706 | CA | VAL | 211 | −12.785 | 14.491 | 17.366 | 1.00 | 0.59 | 1SG | 1707 |
| ATOM | 1707 | CB | VAL | 211 | −12.071 | 13.492 | 16.490 | 1.00 | 0.59 | 1SG | 1708 |
| ATOM | 1708 | CG1 | VAL | 211 | −13.047 | 12.642 | 15.660 | 1.00 | 0.59 | 1SG | 1709 |
| ATOM | 1709 | CG2 | VAL | 211 | −11.082 | 14.291 | 15.635 | 1.00 | 0.59 | 1SG | 1710 |
| ATOM | 1710 | C | VAL | 211 | −13.389 | 15.566 | 16.526 | 1.00 | 0.59 | 1SG | 1711 |
| ATOM | 1711 | O | VAL | 211 | −13.170 | 16.747 | 16.780 | 1.00 | 0.59 | 1SG | 1712 |
| ATOM | 1712 | N | LYS | 212 | −14.184 | 15.207 | 15.508 | 1.00 | 0.48 | 1SG | 1713 |
| ATOM | 1713 | CA | LYS | 212 | −14.648 | 16.231 | 14.631 | 1.00 | 0.48 | 1SG | 1714 |
| ATOM | 1714 | CB | LYS | 212 | −15.249 | 15.741 | 13.299 | 1.00 | 0.48 | 1SG | 1715 |
| ATOM | 1715 | CG | LYS | 212 | −15.694 | 16.948 | 12.466 | 1.00 | 0.48 | 1SG | 1716 |
| ATOM | 1716 | CD | LYS | 212 | −16.041 | 16.677 | 11.004 | 1.00 | 0.48 | 1SG | 1717 |
| ATOM | 1717 | CE | LYS | 212 | −16.493 | 17.931 | 10.250 | 1.00 | 0.48 | 1SG | 1718 |
| ATOM | 1718 | NZ | LYS | 212 | −16.599 | 17.624 | 8.808 | 1.00 | 0.48 | 1SG | 1719 |
| ATOM | 1719 | C | LYS | 212 | −15.684 | 17.095 | 15.268 | 1.00 | 0.48 | 1SG | 1720 |
| ATOM | 1720 | O | LYS | 212 | −15.641 | 18.315 | 15.142 | 1.00 | 0.48 | 1SG | 1721 |
| ATOM | 1721 | N | PRO | 213 | −16.638 | 16.531 | 15.943 | 1.00 | 0.55 | 1SG | 1722 |
| ATOM | 1722 | CA | PRO | 213 | −17.685 | 17.382 | 16.421 | 1.00 | 0.55 | 1SG | 1723 |
| ATOM | 1723 | CD | PRO | 213 | −17.131 | 15.199 | 15.630 | 1.00 | 0.55 | 1SG | 1724 |
| ATOM | 1724 | CB | PRO | 213 | −18.804 | 16.459 | 16.896 | 1.00 | 0.55 | 1SG | 1725 |
| ATOM | 1725 | CG | PRO | 213 | −18.635 | 15.234 | 15.979 | 1.00 | 0.55 | 1SG | 1726 |
| ATOM | 1726 | C | PRO | 213 | −17.276 | 18.475 | 17.350 | 1.00 | 0.55 | 1SG | 1727 |
| ATOM | 1727 | O | PRO | 213 | −17.592 | 19.620 | 17.032 | 1.00 | 0.55 | 1SG | 1728 |
| ATOM | 1728 | N | PRO | 214 | −16.601 | 18.230 | 18.441 | 1.00 | 0.50 | 1SG | 1729 |
| ATOM | 1729 | CA | PRO | 214 | −16.248 | 19.368 | 19.234 | 1.00 | 0.50 | 1SG | 1730 |
| ATOM | 1730 | CD | PRO | 214 | −16.908 | 17.082 | 19.279 | 1.00 | 0.50 | 1SG | 1731 |
| ATOM | 1731 | CB | PRO | 214 | −15.962 | 18.841 | 20.638 | 1.00 | 0.50 | 1SG | 1732 |
| ATOM | 1732 | CG | PRO | 214 | −16.833 | 17.576 | 20.730 | 1.00 | 0.50 | 1SG | 1733 |
| ATOM | 1733 | C | PRO | 214 | −15.125 | 20.162 | 18.661 | 1.00 | 0.50 | 1SG | 1734 |
| ATOM | 1734 | O | PRO | 214 | −15.118 | 21.378 | 18.837 | 1.00 | 0.50 | 1SG | 1735 |
| ATOM | 1735 | N | ASN | 215 | −14.165 | 19.489 | 17.999 | 1.00 | 0.32 | 1SG | 1736 |
| ATOM | 1736 | CA | ASN | 215 | −12.983 | 20.126 | 17.490 | 1.00 | 0.32 | 1SG | 1737 |
| ATOM | 1737 | CB | ASN | 215 | −11.863 | 19.126 | 17.160 | 1.00 | 0.32 | 1SG | 1738 |
| ATOM | 1738 | CG | ASN | 215 | −11.365 | 18.513 | 18.462 | 1.00 | 0.32 | 1SG | 1739 |
| ATOM | 1739 | OD1 | ASN | 215 | −11.203 | 17.298 | 18.568 | 1.00 | 0.32 | 1SG | 1740 |
| ATOM | 1740 | ND2 | ASN | 215 | −11.112 | 19.378 | 19.480 | 1.00 | 0.32 | 1SG | 1741 |
| ATOM | 1741 | C | ASN | 215 | −13.237 | 20.931 | 16.251 | 1.00 | 0.32 | 1SG | 1742 |
| ATOM | 1742 | O | ASN | 215 | −12.657 | 22.001 | 16.077 | 1.00 | 0.32 | 1SG | 1743 |
| ATOM | 1743 | N | SER | 216 | −14.135 | 20.452 | 15.372 | 1.00 | 0.37 | 1SG | 1744 |
| ATOM | 1744 | CA | SER | 216 | −14.280 | 21.006 | 14.053 | 1.00 | 0.37 | 1SG | 1745 |
| ATOM | 1745 | CB | SER | 216 | −15.468 | 20.398 | 13.284 | 1.00 | 0.37 | 1SG | 1746 |
| ATOM | 1746 | OG | SER | 216 | −16.688 | 20.749 | 13.919 | 1.00 | 0.37 | 1SG | 1747 |
| ATOM | 1747 | C | SER | 216 | −14.475 | 22.489 | 14.079 | 1.00 | 0.37 | 1SG | 1748 |
| ATOM | 1748 | O | SER | 216 | −13.713 | 23.216 | 13.447 | 1.00 | 0.37 | 1SG | 1749 |
| ATOM | 1749 | N | LYS | 217 | −15.473 | 23.000 | 14.820 | 1.00 | 0.52 | 1SG | 1750 |
| ATOM | 1750 | CA | LYS | 217 | −15.689 | 24.417 | 14.745 | 1.00 | 0.52 | 1SG | 1751 |
| ATOM | 1751 | CB | LYS | 217 | −16.947 | 24.924 | 15.480 | 1.00 | 0.52 | 1SG | 1752 |
| ATOM | 1752 | CG | LYS | 217 | −17.091 | 24.528 | 16.954 | 1.00 | 0.52 | 1SG | 1753 |
| ATOM | 1753 | CD | LYS | 217 | −17.441 | 23.056 | 17.181 | 1.00 | 0.52 | 1SG | 1754 |
| ATOM | 1754 | CE | LYS | 217 | −17.936 | 22.769 | 18.601 | 1.00 | 0.52 | 1SG | 1755 |
| ATOM | 1755 | NZ | LYS | 217 | −18.688 | 21.495 | 18.630 | 1.00 | 0.52 | 1SG | 1756 |
| ATOM | 1756 | C | LYS | 217 | −14.498 | 25.143 | 15.299 | 1.00 | 0.52 | 1SG | 1757 |
| ATOM | 1757 | O | LYS | 217 | −14.104 | 26.185 | 14.779 | 1.00 | 0.52 | 1SG | 1758 |
| ATOM | 1758 | N | LEU | 218 | −13.903 | 24.592 | 16.366 | 1.00 | 0.42 | 1SG | 1759 |
| ATOM | 1759 | CA | LEU | 218 | −12.816 | 25.186 | 17.082 | 1.00 | 0.42 | 1SG | 1760 |
| ATOM | 1760 | CB | LEU | 218 | −12.474 | 24.425 | 18.377 | 1.00 | 0.42 | 1SG | 1761 |
| ATOM | 1761 | CG | LEU | 218 | −13.603 | 24.425 | 19.426 | 1.00 | 0.42 | 1SG | 1762 |
| ATOM | 1762 | CD2 | LEU | 218 | −14.081 | 25.852 | 19.739 | 1.00 | 0.42 | 1SG | 1763 |
| ATOM | 1763 | CD1 | LEU | 218 | −13.188 | 23.646 | 20.687 | 1.00 | 0.42 | 1SG | 1764 |
| ATOM | 1764 | C | LEU | 218 | −11.540 | 25.242 | 16.285 | 1.00 | 0.42 | 1SG | 1765 |
| ATOM | 1765 | O | LEU | 218 | −10.763 | 26.179 | 16.472 | 1.00 | 0.42 | 1SG | 1766 |
| ATOM | 1766 | N | GLU | 219 | −11.303 | 24.251 | 15.391 | 1.00 | 0.53 | 1SG | 1767 |
| ATOM | 1767 | CA | GLU | 219 | −10.058 | 24.030 | 14.687 | 1.00 | 0.53 | 1SG | 1768 |
| ATOM | 1768 | CB | GLU | 219 | −10.188 | 23.204 | 13.399 | 1.00 | 0.53 | 1SG | 1769 |
| ATOM | 1769 | CG | GLU | 219 | −8.833 | 23.017 | 12.721 | 1.00 | 0.53 | 1SG | 1770 |
| ATOM | 1770 | CD | GLU | 219 | −7.917 | 22.343 | 13.737 | 1.00 | 0.53 | 1SG | 1771 |
| ATOM | 1771 | OE1 | GLU | 219 | −8.435 | 21.545 | 14.563 | 1.00 | 0.53 | 1SG | 1772 |
| ATOM | 1772 | OE2 | GLU | 219 | −6.689 | 22.624 | 13.706 | 1.00 | 0.53 | 1SG | 1773 |
| ATOM | 1773 | C | GLU | 219 | −9.313 | 25.289 | 14.354 | 1.00 | 0.53 | 1SG | 1774 |
| ATOM | 1774 | O | GLU | 219 | −9.837 | 26.207 | 13.726 | 1.00 | 0.53 | 1SG | 1775 |
| ATOM | 1775 | N | ARG | 220 | −8.035 | 25.333 | 14.791 | 1.00 | 0.85 | 1SG | 1776 |
| ATOM | 1776 | CA | ARG | 220 | −7.182 | 26.474 | 14.624 | 1.00 | 0.85 | 1SG | 1777 |
| ATOM | 1777 | CB | ARG | 220 | −6.149 | 26.649 | 15.753 | 1.00 | 0.85 | 1SG | 1778 |
| ATOM | 1778 | CG | ARG | 220 | −6.737 | 27.204 | 17.053 | 1.00 | 0.85 | 1SG | 1779 |
| ATOM | 1779 | CD | ARG | 220 | −7.788 | 26.303 | 17.705 | 1.00 | 0.85 | 1SG | 1780 |
| ATOM | 1780 | NE | ARG | 220 | −8.219 | 26.965 | 18.969 | 1.00 | 0.85 | 1SG | 1781 |
| ATOM | 1781 | CZ | ARG | 220 | −9.182 | 27.932 | 18.947 | 1.00 | 0.85 | 1SG | 1782 |
| ATOM | 1782 | NH1 | ARG | 220 | −9.749 | 28.309 | 17.763 | 1.00 | 0.85 | 1SG | 1783 |
| ATOM | 1783 | NH2 | ARG | 220 | −9.571 | 28.532 | 20.110 | 1.00 | 0.85 | 1SG | 1784 |
| ATOM | 1784 | C | ARG | 220 | −6.427 | 26.387 | 13.336 | 1.00 | 0.85 | 1SG | 1785 |
| ATOM | 1785 | O | ARG | 220 | −6.435 | 25.371 | 12.643 | 1.00 | 0.85 | 1SG | 1786 |
| ATOM | 1786 | N | PHE | 221 | −5.783 | 27.517 | 12.984 | 1.00 | 0.96 | 1SG | 1787 |
| ATOM | 1787 | CA | PHE | 221 | −4.997 | 27.674 | 11.799 | 1.00 | 0.96 | 1SG | 1788 |
| ATOM | 1788 | CB | PHE | 221 | −4.699 | 29.151 | 11.468 | 1.00 | 0.96 | 1SG | 1789 |
| ATOM | 1789 | CG | PHE | 221 | −4.110 | 29.832 | 12.658 | 1.00 | 0.96 | 1SG | 1790 |
| ATOM | 1790 | CD1 | PHE | 221 | −4.935 | 30.327 | 13.642 | 1.00 | 0.96 | 1SG | 1791 |
| ATOM | 1791 | CD2 | PHE | 221 | −2.751 | 29.995 | 12.790 | 1.00 | 0.96 | 1SG | 1792 |
| ATOM | 1792 | CE1 | PHE | 221 | −4.416 | 30.964 | 14.745 | 1.00 | 0.96 | 1SG | 1793 |
| ATOM | 1793 | CE2 | PHE | 221 | −2.225 | 30.632 | 13.890 | 1.00 | 0.96 | 1SG | 1794 |
| ATOM | 1794 | CZ | PHE | 221 | −3.056 | 31.117 | 14.870 | 1.00 | 0.96 | 1SG | 1795 |
| ATOM | 1795 | C | PHE | 221 | −3.718 | 26.889 | 11.861 | 1.00 | 0.96 | 1SG | 1796 |
| ATOM | 1796 | O | PHE | 221 | −3.278 | 26.365 | 10.840 | 1.00 | 0.96 | 1SG | 1797 |
| ATOM | 1797 | N | THR | 222 | −3.065 | 26.786 | 13.039 | 1.00 | 0.85 | 1SG | 1798 |
| ATOM | 1798 | CA | THR | 222 | −1.808 | 26.083 | 13.061 | 1.00 | 0.85 | 1SG | 1799 |
| ATOM | 1799 | CB | THR | 222 | −0.626 | 26.988 | 13.051 | 1.00 | 0.85 | 1SG | 1800 |
| ATOM | 1800 | OG1 | THR | 222 | −0.565 | 27.723 | 14.263 | 1.00 | 0.85 | 1SG | 1801 |
| ATOM | 1801 | CG2 | THR | 222 | −0.816 | 27.957 | 11.895 | 1.00 | 0.85 | 1SG | 1802 |
| ATOM | 1802 | C | THR | 222 | −1.653 | 25.343 | 14.345 | 1.00 | 0.85 | 1SG | 1803 |
| ATOM | 1803 | O | THR | 222 | −2.436 | 25.511 | 15.278 | 1.00 | 0.85 | 1SG | 1804 |
| ATOM | 1804 | N | TYR | 223 | −0.589 | 24.514 | 14.398 | 1.00 | 0.64 | 1SG | 1805 |
| ATOM | 1805 | CA | TYR | 223 | −0.211 | 23.765 | 15.561 | 1.00 | 0.64 | 1SG | 1806 |
| ATOM | 1806 | CB | TYR | 223 | 0.473 | 24.628 | 16.634 | 1.00 | 0.64 | 1SG | 1807 |
| ATOM | 1807 | CG | TYR | 223 | 1.729 | 25.156 | 16.037 | 1.00 | 0.64 | 1SG | 1808 |
| ATOM | 1808 | CD1 | TYR | 223 | 2.819 | 24.334 | 15.889 | 1.00 | 0.64 | 1SG | 1809 |
| ATOM | 1809 | CD2 | TYR | 223 | 1.824 | 26.468 | 15.629 | 1.00 | 0.64 | 1SG | 1810 |
| ATOM | 1810 | CE1 | TYR | 223 | 3.986 | 24.813 | 15.344 | 1.00 | 0.64 | 1SG | 1811 |
| ATOM | 1811 | CE2 | TYR | 223 | 2.989 | 26.953 | 15.083 | 1.00 | 0.64 | 1SG | 1812 |
| ATOM | 1812 | CZ | TYR | 223 | 4.074 | 26.123 | 14.941 | 1.00 | 0.64 | 1SG | 1813 |
| ATOM | 1813 | OH | TYR | 223 | 5.276 | 26.612 | 14.386 | 1.00 | 0.64 | 1SG | 1814 |
| ATOM | 1814 | C | TYR | 223 | −1.418 | 23.133 | 16.177 | 1.00 | 0.64 | 1SG | 1815 |
| ATOM | 1815 | O | TYR | 223 | −1.817 | 23.480 | 17.289 | 1.00 | 0.64 | 1SG | 1816 |
| ATOM | 1816 | N | HIS | 224 | −2.021 | 22.165 | 15.465 | 1.00 | 0.53 | 1SG | 1817 |
| ATOM | 1817 | CA | HIS | 224 | −3.214 | 21.529 | 15.940 | 1.00 | 0.53 | 1SG | 1818 |
| ATOM | 1818 | ND1 | HIS | 224 | −2.920 | 18.187 | 15.263 | 1.00 | 0.53 | 1SG | 1819 |
| ATOM | 1819 | CG | HIS | 224 | −2.914 | 19.391 | 14.592 | 1.00 | 0.53 | 1SG | 1820 |
| ATOM | 1820 | CB | HIS | 224 | −3.815 | 20.543 | 14.921 | 1.00 | 0.53 | 1SG | 1821 |
| ATOM | 1821 | NE2 | HIS | 224 | −1.372 | 18.026 | 13.679 | 1.00 | 0.53 | 1SG | 1822 |
| ATOM | 1822 | CD2 | HIS | 224 | −1.962 | 19.272 | 13.626 | 1.00 | 0.53 | 1SG | 1823 |
| ATOM | 1823 | CE1 | HIS | 224 | −1.981 | 17.407 | 14.677 | 1.00 | 0.53 | 1SG | 1824 |
| ATOM | 1824 | C | HIS | 224 | −2.920 | 20.779 | 17.203 | 1.00 | 0.53 | 1SG | 1825 |
| ATOM | 1825 | O | HIS | 224 | −3.720 | 20.781 | 18.136 | 1.00 | 0.53 | 1SG | 1826 |
| ATOM | 1826 | N | HIS | 225 | −1.746 | 20.131 | 17.273 | 1.00 | 0.48 | 1SG | 1827 |
| ATOM | 1827 | CA | HIS | 225 | −1.387 | 19.301 | 18.388 | 1.00 | 0.48 | 1SG | 1828 |
| ATOM | 1828 | ND1 | HIS | 225 | 0.953 | 19.845 | 16.129 | 1.00 | 0.48 | 1SG | 1829 |
| ATOM | 1829 | CG | HIS | 225 | 0.930 | 19.638 | 17.491 | 1.00 | 0.48 | 1SG | 1830 |
| ATOM | 1830 | CB | HIS | 225 | −0.701 | 18.676 | 18.168 | 1.00 | 0.48 | 1SG | 1831 |
| ATOM | 1831 | NE2 | HIS | 225 | 2.476 | 21.207 | 17.004 | 1.00 | 0.48 | 1SG | 1832 |
| ATOM | 1832 | CD2 | HIS | 225 | 1.868 | 20.477 | 18.010 | 1.00 | 0.48 | 1SG | 1833 |
| ATOM | 1833 | CE1 | HIS | 225 | 1.894 | 20.793 | 15.893 | 1.00 | 0.48 | 1SG | 1834 |
| ATOM | 1834 | C | HIS | 225 | −1.356 | 20.082 | 19.663 | 1.00 | 0.48 | 1SG | 1835 |
| ATOM | 1835 | O | HIS | 225 | −1.953 | 19.669 | 20.657 | 1.00 | 0.48 | 1SG | 1836 |
| ATOM | 1836 | N | GLU | 226 | −0.676 | 21.243 | 19.669 | 1.00 | 0.48 | 1SG | 1837 |
| ATOM | 1837 | CA | GLU | 226 | −0.549 | 22.010 | 20.872 | 1.00 | 0.48 | 1SG | 1838 |
| ATOM | 1838 | CB | GLU | 226 | 0.442 | 23.179 | 20.751 | 1.00 | 0.48 | 1SG | 1839 |
| ATOM | 1839 | CG | GLU | 226 | 0.066 | 24.226 | 19.704 | 1.00 | 0.48 | 1SG | 1840 |
| ATOM | 1840 | CD | GLU | 226 | 1.141 | 25.306 | 19.724 | 1.00 | 0.48 | 1SG | 1841 |
| ATOM | 1841 | OE1 | GLU | 226 | 2.096 | 25.174 | 20.536 | 1.00 | 0.48 | 1SG | 1842 |
| ATOM | 1842 | OE2 | GLU | 226 | 1.023 | 26.278 | 18.931 | 1.00 | 0.48 | 1SG | 1843 |
| ATOM | 1843 | C | GLU | 226 | −1.884 | 22.554 | 21.270 | 1.00 | 0.48 | 1SG | 1844 |
| ATOM | 1844 | O | GLU | 226 | −2.165 | 22.701 | 22.457 | 1.00 | 0.48 | 1SG | 1845 |
| ATOM | 1845 | N | LEU | 227 | −2.740 | 22.874 | 20.281 | 1.00 | 0.57 | 1SG | 1846 |
| ATOM | 1846 | CA | LEU | 227 | −4.011 | 23.480 | 20.553 | 1.00 | 0.57 | 1SG | 1847 |
| ATOM | 1847 | CB | LEU | 227 | −4.796 | 23.818 | 19.270 | 1.00 | 0.57 | 1SG | 1848 |
| ATOM | 1848 | CG | LEU | 227 | −6.054 | 24.692 | 19.481 | 1.00 | 0.57 | 1SG | 1849 |
| ATOM | 1849 | CD2 | LEU | 227 | −5.685 | 26.055 | 20.087 | 1.00 | 0.57 | 1SG | 1850 |
| ATOM | 1850 | CD1 | LEU | 227 | −7.157 | 23.970 | 20.268 | 1.00 | 0.57 | 1SG | 1851 |
| ATOM | 1851 | C | LEU | 227 | −4.840 | 22.542 | 21.375 | 1.00 | 0.57 | 1SG | 1852 |
| ATOM | 1852 | O | LEU | 227 | −5.526 | 22.969 | 22.302 | 1.00 | 0.57 | 1SG | 1853 |
| ATOM | 1853 | N | ARG | 228 | −4.801 | 21.236 | 21.061 | 1.00 | 0.55 | 1SG | 1854 |
| ATOM | 1854 | CA | ARG | 228 | −5.582 | 20.262 | 21.773 | 1.00 | 0.55 | 1SG | 1855 |
| ATOM | 1855 | CB | ARG | 228 | −5.473 | 18.844 | 21.189 | 1.00 | 0.55 | 1SG | 1856 |
| ATOM | 1856 | CG | ARG | 228 | −6.346 | 18.583 | 19.957 | 1.00 | 0.55 | 1SG | 1857 |
| ATOM | 1857 | CD | ARG | 228 | −6.056 | 19.491 | 18.761 | 1.00 | 0.55 | 1SG | 1858 |
| ATOM | 1858 | NE | ARG | 228 | −6.763 | 20.782 | 18.991 | 1.00 | 0.55 | 1SG | 1859 |
| ATOM | 1859 | CZ | ARG | 228 | −8.009 | 20.970 | 18.462 | 1.00 | 0.55 | 1SG | 1860 |
| ATOM | 1860 | NH1 | ARG | 228 | −8.587 | 19.983 | 17.717 | 1.00 | 0.55 | 1SG | 1861 |
| ATOM | 1861 | NH2 | ARG | 228 | −8.679 | 22.140 | 18.669 | 1.00 | 0.55 | 1SG | 1862 |
| ATOM | 1862 | C | ARG | 228 | −5.119 | 20.205 | 23.197 | 1.00 | 0.55 | 1SG | 1863 |
| ATOM | 1863 | O | ARG | 228 | −5.889 | 19.889 | 24.103 | 1.00 | 0.55 | 1SG | 1864 |
| ATOM | 1864 | N | ARG | 229 | −3.827 | 20.493 | 23.423 | 1.00 | 0.49 | 1SG | 1865 |
| ATOM | 1865 | CA | ARG | 229 | −3.220 | 20.426 | 24.721 | 1.00 | 0.49 | 1SG | 1866 |
| ATOM | 1866 | CB | ARG | 229 | −1.704 | 20.670 | 24.663 | 1.00 | 0.49 | 1SG | 1867 |
| ATOM | 1867 | CG | ARG | 229 | −0.989 | 19.616 | 23.816 | 1.00 | 0.49 | 1SG | 1868 |
| ATOM | 1868 | CD | ARG | 229 | 0.524 | 19.806 | 23.721 | 1.00 | 0.49 | 1SG | 1869 |
| ATOM | 1869 | NE | ARG | 229 | 1.046 | 18.707 | 22.862 | 1.00 | 0.49 | 1SG | 1870 |
| ATOM | 1870 | CZ | ARG | 229 | 2.359 | 18.695 | 22.489 | 1.00 | 0.49 | 1SG | 1871 |
| ATOM | 1871 | NH1 | ARG | 229 | 3.210 | 19.656 | 22.952 | 1.00 | 0.49 | 1SG | 1872 |
| ATOM | 1872 | NH2 | ARG | 229 | 2.818 | 17.727 | 21.643 | 1.00 | 0.49 | 1SG | 1873 |
| ATOM | 1873 | C | ARG | 229 | −3.833 | 21.443 | 25.642 | 1.00 | 0.49 | 1SG | 1874 |
| ATOM | 1874 | O | ARG | 229 | −3.856 | 21.241 | 26.856 | 1.00 | 0.49 | 1SG | 1875 |
| ATOM | 1875 | N | VAL | 230 | −4.321 | 22.580 | 25.104 | 1.00 | 0.61 | 1SG | 1876 |
| ATOM | 1876 | CA | VAL | 230 | −4.867 | 23.597 | 25.965 | 1.00 | 0.61 | 1SG | 1877 |
| ATOM | 1877 | CB | VAL | 230 | −5.315 | 24.839 | 25.256 | 1.00 | 0.61 | 1SG | 1878 |
| ATOM | 1878 | CG1 | VAL | 230 | −4.108 | 25.451 | 24.526 | 1.00 | 0.61 | 1SG | 1879 |
| ATOM | 1879 | CG2 | VAL | 230 | −6.507 | 24.492 | 24.358 | 1.00 | 0.61 | 1SG | 1880 |
| ATOM | 1880 | C | VAL | 230 | −6.047 | 23.053 | 26.709 | 1.00 | 0.61 | 1SG | 1881 |
| ATOM | 1881 | O | VAL | 230 | −6.672 | 22.064 | 26.328 | 1.00 | 0.61 | 1SG | 1882 |
| ATOM | 1882 | N | PRO | 231 | −6.337 | 23.711 | 27.801 | 1.00 | 0.61 | 1SG | 1883 |
| ATOM | 1883 | CA | PRO | 231 | −7.411 | 23.278 | 28.653 | 1.00 | 0.61 | 1SG | 1884 |
| ATOM | 1884 | CD | PRO | 231 | −5.290 | 24.388 | 28.551 | 1.00 | 0.61 | 1SG | 1885 |
| ATOM | 1885 | CB | PRO | 231 | −7.226 | 24.024 | 29.973 | 1.00 | 0.61 | 1SG | 1886 |
| ATOM | 1886 | CG | PRO | 231 | −5.713 | 24.295 | 30.026 | 1.00 | 0.61 | 1SG | 1887 |
| ATOM | 1887 | C | PRO | 231 | −8.766 | 23.448 | 28.063 | 1.00 | 0.61 | 1SG | 1888 |
| ATOM | 1888 | O | PRO | 231 | −9.667 | 22.695 | 28.433 | 1.00 | 0.61 | 1SG | 1889 |
| ATOM | 1889 | N | TYR | 232 | −8.951 | 24.420 | 27.156 | 1.00 | 0.40 | 1SG | 1890 |
| ATOM | 1890 | CA | TYR | 232 | −10.271 | 24.596 | 26.642 | 1.00 | 0.40 | 1SG | 1891 |
| ATOM | 1891 | CB | TYR | 232 | −10.369 | 25.745 | 25.622 | 1.00 | 0.40 | 1SG | 1892 |
| ATOM | 1892 | CG | TYR | 232 | −10.109 | 27.016 | 26.355 | 1.00 | 0.40 | 1SG | 1893 |
| ATOM | 1893 | CD1 | TYR | 232 | −8.822 | 27.447 | 26.582 | 1.00 | 0.40 | 1SG | 1894 |
| ATOM | 1894 | CD2 | TYR | 232 | −11.157 | 27.779 | 26.818 | 1.00 | 0.40 | 1SG | 1895 |
| ATOM | 1895 | CE1 | TYR | 232 | −8.585 | 28.621 | 27.260 | 1.00 | 0.40 | 1SG | 1896 |
| ATOM | 1896 | CE2 | TYR | 232 | −10.926 | 28.953 | 27.496 | 1.00 | 0.40 | 1SG | 1897 |
| ATOM | 1897 | CZ | TYR | 232 | −9.638 | 29.375 | 27.718 | 1.00 | 0.40 | 1SG | 1898 |
| ATOM | 1898 | OH | TYR | 232 | −9.398 | 30.579 | 28.414 | 1.00 | 0.40 | 1SG | 1899 |
| ATOM | 1899 | C | TYR | 232 | −10.661 | 23.330 | 25.954 | 1.00 | 0.40 | 1SG | 1900 |
| ATOM | 1900 | O | TYR | 232 | −11.761 | 22.819 | 26.152 | 1.00 | 0.40 | 1SG | 1901 |
| ATOM | 1901 | N | GLU | 233 | −9.768 | 22.801 | 25.098 | 1.00 | 0.46 | 1SG | 1902 |
| ATOM | 1902 | CA | GLU | 233 | −10.080 | 21.598 | 24.385 | 1.00 | 0.46 | 1SG | 1903 |
| ATOM | 1903 | CB | GLU | 233 | −9.184 | 21.385 | 23.155 | 1.00 | 0.46 | 1SG | 1904 |
| ATOM | 1904 | CG | GLU | 233 | −9.660 | 22.183 | 21.939 | 1.00 | 0.46 | 1SG | 1905 |
| ATOM | 1905 | CD | GLU | 233 | −9.682 | 23.660 | 22.296 | 1.00 | 0.46 | 1SG | 1906 |
| ATOM | 1906 | OE1 | GLU | 233 | −8.598 | 24.211 | 22.617 | 1.00 | 0.46 | 1SG | 1907 |
| ATOM | 1907 | OE2 | GLU | 233 | −10.788 | 24.259 | 22.250 | 1.00 | 0.46 | 1SG | 1908 |
| ATOM | 1908 | C | GLU | 233 | −10.035 | 20.356 | 25.227 | 1.00 | 0.46 | 1SG | 1909 |
| ATOM | 1909 | O | GLU | 233 | −10.976 | 19.563 | 25.206 | 1.00 | 0.46 | 1SG | 1910 |
| ATOM | 1910 | N | TYR | 234 | −8.950 | 20.145 | 26.003 | 1.00 | 0.60 | 1SG | 1911 |
| ATOM | 1911 | CA | TYR | 234 | −8.819 | 18.872 | 26.655 | 1.00 | 0.60 | 1SG | 1912 |
| ATOM | 1912 | CB | TYR | 234 | −7.372 | 18.500 | 27.068 | 1.00 | 0.60 | 1SG | 1913 |
| ATOM | 1913 | CG | TYR | 234 | −6.928 | 19.004 | 28.396 | 1.00 | 0.60 | 1SG | 1914 |
| ATOM | 1914 | CD1 | TYR | 234 | −6.877 | 20.342 | 28.694 | 1.00 | 0.60 | 1SG | 1915 |
| ATOM | 1915 | CD2 | TYR | 234 | −6.485 | 18.095 | 29.331 | 1.00 | 0.60 | 1SG | 1916 |
| ATOM | 1916 | CE1 | TYR | 234 | −6.443 | 20.759 | 29.932 | 1.00 | 0.60 | 1SG | 1917 |
| ATOM | 1917 | CE2 | TYR | 234 | −6.051 | 18.506 | 30.568 | 1.00 | 0.60 | 1SG | 1918 |
| ATOM | 1918 | CZ | TYR | 234 | −6.036 | 19.845 | 30.873 | 1.00 | 0.60 | 1SG | 1919 |
| ATOM | 1919 | OH | TYR | 234 | −5.592 | 20.278 | 32.140 | 1.00 | 0.60 | 1SG | 1920 |
| ATOM | 1920 | C | TYR | 234 | −9.770 | 18.739 | 27.797 | 1.00 | 0.60 | 1SG | 1921 |
| ATOM | 1921 | O | TYR | 234 | −10.285 | 17.647 | 28.046 | 1.00 | 0.60 | 1SG | 1922 |
| ATOM | 1922 | N | VAL | 235 | −10.039 | 19.840 | 28.520 | 1.00 | 0.66 | 1SG | 1923 |
| ATOM | 1923 | CA | VAL | 235 | −10.889 | 19.742 | 29.666 | 1.00 | 0.66 | 1SG | 1924 |
| ATOM | 1924 | CB | VAL | 235 | −12.213 | 19.070 | 29.370 | 1.00 | 0.66 | 1SG | 1925 |
| ATOM | 1925 | CG1 | VAL | 235 | −13.091 | 18.996 | 30.635 | 1.00 | 0.66 | 1SG | 1926 |
| ATOM | 1926 | CG2 | VAL | 235 | −12.872 | 19.833 | 28.209 | 1.00 | 0.66 | 1SG | 1927 |
| ATOM | 1927 | C | VAL | 235 | −10.031 | 18.948 | 30.605 | 1.00 | 0.66 | 1SG | 1928 |
| ATOM | 1928 | O | VAL | 235 | −8.816 | 18.946 | 30.464 | 1.00 | 0.66 | 1SG | 1929 |
| ATOM | 1929 | N | LYS | 236 | −10.568 | 18.230 | 31.591 | 1.00 | 0.78 | 1SG | 1930 |
| ATOM | 1930 | CA | LYS | 236 | −9.610 | 17.539 | 32.396 | 1.00 | 0.78 | 1SG | 1931 |
| ATOM | 1931 | CB | LYS | 236 | −9.965 | 17.563 | 33.894 | 1.00 | 0.78 | 1SG | 1932 |
| ATOM | 1932 | CG | LYS | 236 | −9.944 | 18.965 | 34.509 | 1.00 | 0.78 | 1SG | 1933 |
| ATOM | 1933 | CD | LYS | 236 | −8.576 | 19.646 | 34.450 | 1.00 | 0.78 | 1SG | 1934 |
| ATOM | 1934 | CB | LYS | 236 | −8.566 | 21.050 | 35.061 | 1.00 | 0.78 | 1SG | 1935 |
| ATOM | 1935 | NZ | LYS | 236 | −7.195 | 21.607 | 35.035 | 1.00 | 0.78 | 1SG | 1936 |
| ATOM | 1936 | C | LYS | 236 | −9.593 | 16.105 | 31.970 | 1.00 | 0.78 | 1SG | 1937 |
| ATOM | 1937 | O | LYS | 236 | −9.593 | 15.210 | 32.813 | 1.00 | 0.78 | 1SG | 1938 |
| ATOM | 1938 | N | LEU | 237 | −9.546 | 15.842 | 30.648 | 1.00 | 0.70 | 1SG | 1939 |
| ATOM | 1939 | CA | LEU | 237 | −9.586 | 14.480 | 30.190 | 1.00 | 0.70 | 1SG | 1940 |
| ATOM | 1940 | CD | LEU | 237 | −10.394 | 14.293 | 28.894 | 1.00 | 0.70 | 1SG | 1941 |
| ATOM | 1941 | CD | LEU | 237 | −11.892 | 14.622 | 29.053 | 1.00 | 0.70 | 1SG | 1942 |
| ATOM | 1942 | CD2 | LEU | 237 | −12.700 | 14.168 | 27.826 | 1.00 | 0.70 | 1SG | 1943 |
| ATOM | 1943 | CD1 | LEU | 237 | −12.111 | 16.105 | 29.395 | 1.00 | 0.70 | 1SG | 1944 |
| ATOM | 1944 | C | LEU | 237 | −8.196 | 13.985 | 29.928 | 1.00 | 0.70 | 1SG | 1945 |
| ATOM | 1945 | O | LEU | 237 | −7.335 | 14.693 | 29.407 | 1.00 | 0.70 | 1SG | 1946 |
| ATOM | 1946 | N | PRO | 238 | −7.972 | 12.776 | 30.378 | 1.00 | 0.51 | 1SG | 1947 |
| ATOM | 1947 | CA | PRO | 238 | −6.693 | 12.147 | 30.126 | 1.00 | 0.51 | 1SG | 1948 |
| ATOM | 1948 | CD | PRO | 238 | −8.403 | 12.533 | 31.748 | 1.00 | 0.51 | 1SG | 1949 |
| ATOM | 1949 | CB | PRO | 238 | −6.280 | 11.436 | 31.418 | 1.00 | 0.51 | 1SG | 1950 |
| ATOM | 1950 | CG | PRO | 238 | −7.571 | 11.343 | 32.240 | 1.00 | 0.51 | 1SG | 1951 |
| ATOM | 1951 | C | PRO | 238 | −6.632 | 11.241 | 28.924 | 1.00 | 0.51 | 1SG | 1952 |
| ATOM | 1952 | O | PRO | 238 | −7.652 | 10.988 | 28.285 | 1.00 | 0.51 | 1SG | 1953 |
| ATOM | 1953 | N | ILE | 239 | −5.417 | 10.736 | 28.616 | 1.00 | 0.60 | 1SG | 1954 |
| ATOM | 1954 | CA | ILE | 239 | −5.154 | 9.848 | 27.516 | 1.00 | 0.60 | 1SG | 1955 |
| ATOM | 1955 | CB | ILE | 239 | −4.288 | 10.490 | 26.478 | 1.00 | 0.60 | 1SG | 1956 |
| ATOM | 1956 | CG2 | ILE | 239 | −2.942 | 10.819 | 27.143 | 1.00 | 0.60 | 1SG | 1957 |
| ATOM | 1957 | CG1 | ILE | 239 | −4.170 | 9.623 | 25.219 | 1.00 | 0.60 | 1SG | 1958 |
| ATOM | 1958 | CD1 | ILE | 239 | −3.573 | 10.373 | 24.028 | 1.00 | 0.60 | 1SG | 1959 |
| ATOM | 1959 | C | ILE | 239 | −4.384 | 8.692 | 28.073 | 1.00 | 0.60 | 1SG | 1960 |
| ATOM | 1960 | O | ILE | 239 | −3.729 | 8.809 | 29.107 | 1.00 | 0.60 | 1SG | 1961 |
| ATOM | 1961 | N | ARG | 240 | −4.478 | 7.514 | 27.424 | 1.00 | 0.78 | 1SG | 1962 |
| ATOM | 1962 | CA | ARG | 240 | −3.739 | 6.385 | 27.906 | 1.00 | 0.78 | 1SG | 1963 |
| ATOM | 1963 | CB | ARG | 240 | −4.413 | 5.023 | 27.672 | 1.00 | 0.78 | 1SG | 1964 |
| ATOM | 1964 | CG | ARG | 240 | −3.585 | 3.858 | 28.219 | 1.00 | 0.78 | 1SG | 1965 |
| ATOM | 1965 | CD | ARG | 240 | −4.002 | 2.478 | 27.703 | 1.00 | 0.78 | 1SG | 1966 |
| ATOM | 1966 | NE | ARG | 240 | −3.405 | 2.318 | 26.350 | 1.00 | 0.78 | 1SG | 1967 |
| ATOM | 1967 | CZ | ARG | 240 | −2.720 | 1.182 | 26.031 | 1.00 | 0.78 | 1SG | 1968 |
| ATOM | 1968 | NH1 | ARG | 240 | −2.577 | 0.180 | 26.945 | 1.00 | 0.78 | 1SG | 1969 |
| ATOM | 1969 | NH2 | ARG | 240 | −2.163 | 1.048 | 24.796 | 1.00 | 0.78 | 1SG | 1970 |
| ATOM | 1970 | C | ARG | 240 | −2.444 | 6.353 | 27.158 | 1.00 | 0.78 | 1SG | 1971 |
| ATOM | 1971 | O | ARG | 240 | −2.368 | 6.666 | 25.969 | 1.00 | 0.78 | 1SG | 1972 |
| ATOM | 1972 | N | THR | 241 | −1.371 | 5.992 | 27.877 | 1.00 | 0.69 | 1SG | 1973 |
| ATOM | 1973 | CA | THR | 241 | −0.069 | 5.887 | 27.288 | 1.00 | 0.69 | 1SG | 1974 |
| ATOM | 1974 | CB | THR | 241 | 0.846 | 6.993 | 27.691 | 1.00 | 0.69 | 1SG | 1975 |
| ATOM | 1975 | OG1 | THR | 241 | 2.129 | 6.762 | 27.133 | 1.00 | 0.69 | 1SG | 1976 |
| ATOM | 1976 | CG2 | THR | 241 | 0.927 | 7.035 | 29.223 | 1.00 | 0.69 | 1SG | 1977 |
| ATOM | 1977 | C | THR | 241 | 0.548 | 4.623 | 27.799 | 1.00 | 0.69 | 1SG | 1978 |
| ATOM | 1978 | O | THR | 241 | 0.073 | 4.056 | 28.780 | 1.00 | 0.69 | 1SG | 1979 |
| ATOM | 1979 | N | ASN | 242 | 1.628 | 4.142 | 27.146 | 1.00 | 0.41 | 1SG | 1980 |
| ATOM | 1980 | CA | ASN | 242 | 2.216 | 2.907 | 27.597 | 1.00 | 0.41 | 1SG | 1981 |
| ATOM | 1981 | CB | ASN | 242 | 2.356 | 1.870 | 26.465 | 1.00 | 0.41 | 1SG | 1982 |
| ATOM | 1982 | CG | ASN | 242 | 2.505 | 0.466 | 27.040 | 1.00 | 0.41 | 1SG | 1983 |
| ATOM | 1983 | OD1 | ASN | 242 | 3.559 | 0.065 | 27.530 | 1.00 | 0.41 | 1SG | 1984 |
| ATOM | 1984 | ND2 | ASN | 242 | 1.403 | 0.326 | 26.959 | 1.00 | 0.41 | 1SG | 1985 |
| ATOM | 1985 | C | ASN | 242 | 3.584 | 3.197 | 28.143 | 1.00 | 0.41 | 1SG | 1986 |
| ATOM | 1986 | O | ASN | 242 | 4.132 | 4.280 | 27.951 | 1.00 | 0.41 | 1SG | 1987 |
| ATOM | 1987 | N | ILE | 243 | 4.167 | 2.223 | 28.875 | 1.00 | 0.33 | 1SG | 1988 |
| ATOM | 1988 | CA | ILE | 243 | 5.484 | 2.374 | 29.433 | 1.00 | 0.33 | 1SG | 1989 |
| ATOM | 1989 | CB | ILE | 243 | 5.974 | 1.212 | 30.243 | 1.00 | 0.33 | 1SG | 1990 |
| ATOM | 1990 | CG2 | ILE | 243 | 7.466 | 1.453 | 30.523 | 1.00 | 0.33 | 1SG | 1991 |
| ATOM | 1991 | CG2 | ILE | 243 | 5.141 | 1.020 | 31.516 | 1.00 | 0.33 | 1SG | 1992 |
| ATOM | 1992 | CD1 | ILE | 243 | 5.633 | 0.162 | 32.344 | 1.00 | 0.33 | 1SG | 1993 |
| ATOM | 1993 | C | ILE | 243 | 6.447 | 2.507 | 28.297 | 1.00 | 0.33 | 1SG | 1994 |
| ATOM | 1994 | O | ILE | 243 | 7.410 | 3.269 | 28.375 | 1.00 | 0.33 | 1SG | 1995 |
| ATOM | 1995 | N | SER | 244 | 6.233 | 1.721 | 27.226 | 1.00 | 0.63 | 1SG | 1996 |
| ATOM | 1996 | CA | SER | 244 | 7.061 | 1.834 | 26.056 | 1.00 | 0.63 | 1SG | 1997 |
| ATOM | 1997 | CB | SER | 244 | 8.079 | 0.696 | 25.889 | 1.00 | 0.63 | 1SG | 1998 |
| ATOM | 1998 | OG | SER | 244 | 8.827 | 0.880 | 24.698 | 1.00 | 0.63 | 1SG | 1999 |
| ATOM | 1999 | C | SER | 244 | 6.115 | 1.776 | 24.907 | 1.00 | 0.63 | 1SG | 2000 |
| ATOM | 2000 | O | SER | 244 | 5.161 | 1.003 | 24.954 | 1.00 | 0.63 | 1SG | 2001 |
| ATOM | 2001 | N | LYS | 245 | 6.282 | 2.565 | 23.823 | 1.00 | 0.98 | 1SG | 2002 |
| ATOM | 2002 | CA | LYS | 245 | 5.167 | 2.268 | 22.987 | 1.00 | 0.98 | 1SG | 2003 |
| ATOM | 2003 | CB | LYS | 245 | 3.994 | 3.209 | 22.785 | 1.00 | 0.98 | 1SG | 2004 |
| ATOM | 2004 | CG | LYS | 245 | 2.852 | 2.976 | 23.778 | 1.00 | 0.98 | 1SG | 2005 |
| ATOM | 2005 | CD | LYS | 245 | 2.070 | 1.675 | 23.536 | 1.00 | 0.98 | 1SG | 2006 |
| ATOM | 2006 | CB | LYS | 245 | 2.926 | 0.408 | 23.484 | 1.00 | 0.98 | 1SG | 2007 |
| ATOM | 2007 | NZ | LYS | 245 | 2.071 | 0.794 | 23.325 | 1.00 | 0.98 | 1SG | 2008 |
| ATOM | 2008 | C | LYS | 245 | 5.346 | 1.736 | 21.646 | 1.00 | 0.98 | 1SG | 2009 |
| ATOM | 2009 | O | LYS | 245 | 6.276 | 1.013 | 21.306 | 1.00 | 0.98 | 1SG | 2010 |
| ATOM | 2010 | N | GLU | 246 | 4.236 | 1.996 | 20.935 | 1.00 | 0.86 | 1SG | 2011 |
| ATOM | 2011 | CA | GLU | 246 | 3.918 | 1.492 | 19.644 | 1.00 | 0.86 | 1SG | 2012 |
| ATOM | 2012 | CB | GLU | 246 | 2.410 | 1.472 | 19.328 | 1.00 | 0.86 | 1SG | 2013 |
| ATOM | 2013 | CG | GLU | 246 | 2.103 | 0.972 | 17.917 | 1.00 | 0.86 | 1SG | 2014 |
| ATOM | 2014 | CD | GLU | 246 | 2.545 | −0.478 | 17.847 | 1.00 | 0.86 | 1SG | 2015 |
| ATOM | 2015 | OE1 | GLU | 246 | 2.861 | −1.042 | 18.928 | 1.00 | 0.86 | 1SG | 2016 |
| ATOM | 2016 | OE2 | GLU | 246 | 2.588 | −1.037 | 16.719 | 1.00 | 0.86 | 1SG | 2017 |
| ATOM | 2017 | C | GLU | 246 | 4.641 | 2.094 | 18.482 | 1.00 | 0.86 | 1SG | 2018 |
| ATOM | 2018 | O | GLU | 246 | 4.826 | 1.388 | 17.494 | 1.00 | 0.86 | 1SG | 2019 |
| ATOM | 2019 | N | ALA | 247 | 5.024 | 3.390 | 18.470 | 1.00 | 0.46 | 1SG | 2020 |
| ATOM | 2020 | CA | ALA | 247 | 5.659 | 3.715 | 17.214 | 1.00 | 0.46 | 1SG | 2021 |
| ATOM | 2021 | CB | ALA | 247 | 4.711 | 4.330 | 16.175 | 1.00 | 0.46 | 1SG | 2022 |
| ATOM | 2022 | C | ALA | 247 | 6.815 | 4.657 | 17.342 | 1.00 | 0.46 | 1SG | 2023 |
| ATOM | 2023 | O | ALA | 247 | 6.742 | 5.628 | 18.075 | 1.00 | 0.46 | 1SG | 2024 |
| ATOM | 2024 | N | PRO | 248 | 7.922 | 4.412 | 16.693 | 1.00 | 0.44 | 1SG | 2025 |
| ATOM | 2025 | CA | PRO | 248 | 8.969 | 5.407 | 16.764 | 1.00 | 0.44 | 1SG | 2026 |
| ATOM | 2026 | CD | PRO | 248 | 8.468 | 3.066 | 16.708 | 1.00 | 0.44 | 1SG | 2027 |
| ATOM | 2027 | CB | PRO | 248 | 10.269 | 4.644 | 17.031 | 1.00 | 0.44 | 1SG | 2028 |
| ATOM | 2028 | CG | PRO | 248 | 9.979 | 3.234 | 16.493 | 1.00 | 0.44 | 1SG | 2029 |
| ATOM | 2029 | C | PRO | 248 | 9.033 | 6.216 | 15.494 | 1.00 | 0.44 | 1SG | 2030 |
| ATOM | 2030 | O | PRO | 248 | 8.766 | 5.639 | 14.442 | 1.00 | 0.44 | 1SG | 2031 |
| ATOM | 2031 | N | PRO | 249 | 9.311 | 7.497 | 15.523 | 1.00 | 0.66 | 1SG | 2032 |
| ATOM | 2032 | CA | PRO | 249 | 9.547 | 8.153 | 14.254 | 1.00 | 0.66 | 1SG | 2033 |
| ATOM | 2033 | CD | PRO | 249 | 8.420 | 8.296 | 16.348 | 1.00 | 0.66 | 1SG | 2034 |
| ATOM | 2034 | CB | PRO | 249 | 9.028 | 9.576 | 14.381 | 1.00 | 0.66 | 1SG | 2035 |
| ATOM | 2035 | CG | PRO | 249 | 7.949 | 9.466 | 15.469 | 1.00 | 0.66 | 1SG | 2036 |
| ATOM | 2036 | C | PRO | 249 | 10.987 | 8.062 | 13.822 | 1.00 | 0.66 | 1SG | 2037 |
| ATOM | 2037 | O | PRO | 249 | 11.865 | 8.414 | 14.610 | 1.00 | 0.66 | 1SG | 2038 |
| ATOM | 2038 | N | HIS | 250 | 11.257 | 7.574 | 12.592 | 1.00 | 0.63 | 1SG | 2039 |
| ATOM | 2039 | CA | HIS | 250 | 12.605 | 7.413 | 12.111 | 1.00 | 0.63 | 1SG | 2040 |
| ATOM | 2040 | ND1 | HIS | 250 | 15.124 | 6.211 | 10.198 | 1.00 | 0.63 | 1SG | 2041 |
| ATOM | 2041 | CG | HIS | 250 | 14.214 | 5.743 | 11.118 | 1.00 | 0.63 | 1SG | 2042 |
| ATOM | 2042 | CB | HIS | 250 | 12.790 | 6.194 | 11.205 | 1.00 | 0.63 | 1SG | 2043 |
| ATOM | 2043 | NE2 | HIS | 250 | 16.200 | 4.728 | 11.458 | 1.00 | 0.63 | 1SG | 2044 |
| ATOM | 2044 | CD2 | HIS | 250 | 14.888 | 4.838 | 11.880 | 1.00 | 0.63 | 1SG | 2045 |
| ATOM | 2045 | CE1 | HIS | 250 | 16.294 | 5.571 | 10.446 | 1.00 | 0.63 | 1SG | 2046 |
| ATOM | 2046 | C | HIS | 250 | 13.212 | 8.577 | 11.378 | 1.00 | 0.63 | 1SG | 2047 |
| ATOM | 2047 | O | HIS | 250 | 14.425 | 8.761 | 11.438 | 1.00 | 0.63 | 1SG | 2048 |
| ATOM | 2048 | N | ASN | 251 | 12.434 | 9.402 | 10.648 | 1.00 | 0.64 | 1SG | 2049 |
| ATOM | 2049 | CA | ASN | 251 | 13.163 | 10.250 | 9.740 | 1.00 | 0.64 | 1SG | 2050 |
| ATOM | 2050 | CB | ASN | 251 | 12.365 | 10.770 | 8.539 | 1.00 | 0.64 | 1SG | 2051 |
| ATOM | 2051 | CG | ASN | 251 | 13.380 | 11.158 | 7.463 | 1.00 | 0.64 | 1SG | 2052 |
| ATOM | 2052 | OD1 | ASN | 251 | 13.018 | 11.675 | 6.407 | 1.00 | 0.64 | 1SG | 2053 |
| ATOM | 2053 | ND2 | ASN | 251 | 14.688 | 10.882 | 7.719 | 1.00 | 0.64 | 1SG | 2054 |
| ATOM | 2054 | C | ASN | 251 | 13.858 | 11.387 | 10.415 | 1.00 | 0.64 | 1SG | 2055 |
| ATOM | 2055 | O | ASN | 251 | 13.282 | 12.429 | 10.730 | 1.00 | 0.64 | 1SG | 2056 |
| ATOM | 2056 | N | ILE | 252 | 15.163 | 11.154 | 10.648 | 1.00 | 0.77 | 1SG | 2057 |
| ATOM | 2057 | CA | ILE | 252 | 16.127 | 12.022 | 11.260 | 1.00 | 0.77 | 1SG | 2058 |
| ATOM | 2058 | CB | ILE | 252 | 17.364 | 11.281 | 11.673 | 1.00 | 0.77 | 1SG | 2059 |
| ATOM | 2059 | CG2 | ILE | 252 | 18.010 | 10.696 | 10.407 | 1.00 | 0.77 | 1SG | 2060 |
| ATOM | 2060 | CG1 | ILE | 252 | 18.298 | 12.191 | 12.488 | 1.00 | 0.77 | 1SG | 2061 |
| ATOM | 2061 | CD1 | ILE | 252 | 17.714 | 12.577 | 13.844 | 1.00 | 0.77 | 1SG | 2062 |
| ATOM | 2062 | C | ILE | 252 | 16.590 | 13.130 | 10.362 | 1.00 | 0.77 | 1SG | 2063 |
| ATOM | 2063 | O | ILE | 252 | 16.925 | 14.204 | 10.854 | 1.00 | 0.77 | 1SG | 2064 |
| ATOM | 2064 | N | GLN | 253 | 16.656 | 12.905 | 9.034 | 1.00 | 0.81 | 1SG | 2065 |
| ATOM | 2065 | CA | GLN | 253 | 17.328 | 13.868 | 8.204 | 1.00 | 0.81 | 1SG | 2066 |
| ATOM | 2066 | CB | GLN | 253 | 17.358 | 13.489 | 6.710 | 1.00 | 0.81 | 1SG | 2067 |
| ATOM | 2067 | CG | GLN | 253 | 18.297 | 14.382 | 5.891 | 1.00 | 0.81 | 1SG | 2068 |
| ATOM | 2068 | CD | GLN | 253 | 18.537 | 13.732 | 4.533 | 1.00 | 0.81 | 1SG | 2069 |
| ATOM | 2069 | OE1 | GLN | 253 | 19.679 | 13.546 | 4.113 | 1.00 | 0.81 | 1SG | 2070 |
| ATOM | 2070 | NE2 | GLN | 253 | 17.432 | 13.383 | 3.821 | 1.00 | 0.81 | 1SG | 2071 |
| ATOM | 2071 | C | GLN | 253 | 16.761 | 15.249 | 8.334 | 1.00 | 0.81 | 1SG | 2072 |
| ATOM | 2072 | O | GLN | 253 | 17.387 | 16.106 | 8.958 | 1.00 | 0.81 | 1SG | 2073 |
| ATOM | 2073 | N | ILE | 254 | 15.558 | 15.515 | 7.786 | 1.00 | 0.85 | 1SG | 2074 |
| ATOM | 2074 | CA | ILE | 254 | 15.104 | 16.874 | 7.892 | 1.00 | 0.85 | 1SG | 2075 |
| ATOM | 2075 | CB | ILE | 254 | 16.088 | 17.851 | 7.295 | 1.00 | 0.85 | 1SG | 2076 |
| ATOM | 2076 | CG2 | ILE | 254 | 16.202 | 17.541 | 5.793 | 1.00 | 0.85 | 1SG | 2077 |
| ATOM | 2077 | CG1 | ILE | 254 | 15.720 | 19.310 | 7.607 | 1.00 | 0.85 | 1SG | 2078 |
| ATOM | 2078 | CD1 | ILE | 254 | 16.809 | 20.301 | 7.192 | 1.00 | 0.85 | 1SG | 2079 |
| ATOM | 2079 | C | ILE | 254 | 13.835 | 17.039 | 7.118 | 1.00 | 0.85 | 1SG | 2080 |
| ATOM | 2080 | O | ILE | 254 | 13.462 | 16.185 | 6.315 | 1.00 | 0.85 | 1SG | 2081 |
| ATOM | 2081 | N | PHE | 255 | 13.136 | 18.167 | 7.363 | 1.00 | 0.90 | 1SG | 2082 |
| ATOM | 2082 | CA | PHE | 255 | 11.995 | 18.534 | 6.581 | 1.00 | 0.90 | 1SG | 2083 |
| ATOM | 2083 | CB | PHE | 255 | 10.939 | 17.456 | 6.331 | 1.00 | 0.90 | 1SG | 2084 |
| ATOM | 2084 | CG | PHE | 255 | 10.240 | 17.920 | 5.093 | 1.00 | 0.90 | 1SG | 2085 |
| ATOM | 2085 | CD1 | PHE | 255 | 10.929 | 17.931 | 3.899 | 1.00 | 0.90 | 1SG | 2086 |
| ATOM | 2086 | CD2 | PHE | 255 | 8.953 | 18.405 | 5.112 | 1.00 | 0.90 | 1SG | 2087 |
| ATOM | 2087 | CE1 | PHE | 255 | 10.332 | 18.338 | 2.733 | 1.00 | 0.90 | 1SG | 2088 |
| ATOM | 2088 | CE2 | PHE | 255 | 8.347 | 18.822 | 3.950 | 1.00 | 0.90 | 1SG | 2089 |
| ATOM | 2089 | CZ | PHE | 255 | 9.031 | 18.779 | 2.758 | 1.00 | 0.90 | 1SG | 2090 |
| ATOM | 2090 | C | PHE | 255 | 11.365 | 19.748 | 7.182 | 1.00 | 0.90 | 1SG | 2091 |
| ATOM | 2091 | O | PHE | 255 | 12.059 | 20.725 | 7.452 | 1.00 | 0.90 | 1SG | 2092 |
| ATOM | 2092 | N | VAL | 256 | 10.028 | 19.739 | 7.365 | 1.00 | 0.79 | 1SG | 2093 |
| ATOM | 2093 | CA | VAL | 256 | 9.332 | 20.902 | 7.854 | 1.00 | 0.79 | 1SG | 2094 |
| ATOM | 2094 | CB | VAL | 256 | 7.841 | 20.829 | 7.673 | 1.00 | 0.79 | 1SG | 2095 |
| ATOM | 2095 | CG1 | VAL | 256 | 7.532 | 20.790 | 6.169 | 1.00 | 0.79 | 1SG | 2096 |
| ATOM | 2096 | CG2 | VAL | 256 | 7.305 | 19.613 | 8.446 | 1.00 | 0.79 | 1SG | 2097 |
| ATOM | 2097 | C | VAL | 256 | 9.584 | 21.083 | 9.314 | 1.00 | 0.79 | 1SG | 2098 |
| ATOM | 2098 | O | VAL | 256 | 8.661 | 21.366 | 10.074 | 1.00 | 0.79 | 1SG | 2099 |
| ATOM | 2099 | N | GLY | 257 | 10.842 | 20.947 | 9.756 | 1.00 | 0.59 | 1SG | 2100 |
| ATOM | 2100 | CA | GLY | 257 | 11.083 | 21.128 | 11.151 | 1.00 | 0.59 | 1SG | 2101 |
| ATOM | 2101 | C | GLY | 257 | 12.556 | 20.991 | 11.360 | 1.00 | 0.59 | 1SG | 2102 |
| ATOM | 2102 | O | GLY | 257 | 13.251 | 20.351 | 10.572 | 1.00 | 0.59 | 1SG | 2103 |
| ATOM | 2103 | N | SER | 258 | 13.064 | 21.599 | 12.449 | 1.00 | 0.44 | 1SG | 2104 |
| ATOM | 2104 | CA | SER | 258 | 14.465 | 21.534 | 12.747 | 1.00 | 0.44 | 1SG | 2105 |
| ATOM | 2105 | CB | SER | 258 | 14.914 | 22.495 | 13.868 | 1.00 | 0.44 | 1SG | 2106 |
| ATOM | 2106 | OG | SER | 258 | 16.306 | 22.357 | 14.115 | 1.00 | 0.44 | 1SG | 2107 |
| ATOM | 2107 | C | SER | 258 | 14.780 | 20.141 | 13.198 | 1.00 | 0.44 | 1SG | 2108 |
| ATOM | 2108 | O | SER | 258 | 13.910 | 19.400 | 13.654 | 1.00 | 0.44 | 1SG | 2109 |
| ATOM | 2109 | N | ALA | 259 | 16.056 | 19.748 | 13.020 | 1.00 | 0.36 | 1SG | 2110 |
| ATOM | 2110 | CA | ALA | 259 | 16.571 | 18.464 | 13.392 | 1.00 | 0.36 | 1SG | 2111 |
| ATOM | 2111 | CB | ALA | 259 | 17.983 | 18.215 | 12.839 | 1.00 | 0.36 | 1SG | 2112 |
| ATOM | 2112 | C | ALA | 259 | 16.664 | 18.339 | 14.876 | 1.00 | 0.36 | 1SG | 2113 |
| ATOM | 2113 | O | ALA | 259 | 16.465 | 17.258 | 15.419 | 1.00 | 0.36 | 1SG | 2114 |
| ATOM | 2114 | N | TYR | 260 | 16.931 | 19.452 | 15.578 | 1.00 | 0.45 | 1SG | 2115 |
| ATOM | 2115 | CA | TYR | 260 | 17.282 | 19.416 | 16.969 | 1.00 | 0.45 | 1SG | 2116 |
| ATOM | 2116 | CB | TYR | 260 | 17.388 | 20.834 | 17.573 | 1.00 | 0.45 | 1SG | 2117 |
| ATOM | 2117 | CG | TYR | 260 | 17.751 | 20.704 | 19.015 | 1.00 | 0.45 | 1SG | 2118 |
| ATOM | 2118 | CD1 | TYR | 260 | 16.787 | 20.523 | 19.979 | 1.00 | 0.45 | 1SG | 2119 |
| ATOM | 2119 | CD2 | TYR | 260 | 19.071 | 20.773 | 19.401 | 1.00 | 0.45 | 1SG | 2120 |
| ATOM | 2120 | CE1 | TYR | 260 | 17.125 | 20.399 | 21.304 | 1.00 | 0.45 | 1SG | 2121 |
| ATOM | 2121 | CE2 | TYR | 260 | 19.419 | 20.650 | 20.726 | 1.00 | 0.45 | 1SG | 2122 |
| ATOM | 2122 | CZ | TYR | 260 | 18.444 | 20.460 | 21.677 | 1.00 | 0.45 | 1SG | 2123 |
| ATOM | 2123 | OH | TYR | 260 | 18.797 | 20.332 | 23.037 | 1.00 | 0.45 | 1SG | 2124 |
| ATOM | 2124 | C | TYR | 260 | 16.287 | 18.637 | 17.780 | 1.00 | 0.45 | 1SG | 2125 |
| ATOM | 2125 | O | TYR | 260 | 16.677 | 17.766 | 18.556 | 1.00 | 0.45 | 1SG | 2126 |
| ATOM | 2126 | N | PHE | 261 | 14.979 | 18.897 | 17.622 | 1.00 | 0.54 | 1SG | 2127 |
| ATOM | 2127 | CA | PHE | 261 | 14.025 | 18.214 | 18.454 | 1.00 | 0.54 | 1SG | 2128 |
| ATOM | 2128 | CB | PHE | 261 | 12.578 | 18.637 | 18.142 | 1.00 | 0.54 | 1SG | 2129 |
| ATOM | 2129 | CG | PHE | 261 | 11.655 | 17.816 | 18.978 | 1.00 | 0.54 | 1SG | 2130 |
| ATOM | 2130 | CD1 | PHE | 261 | 11.197 | 16.601 | 18.520 | 1.00 | 0.54 | 1SG | 2131 |
| ATOM | 2131 | CD2 | PHE | 261 | 11.245 | 18.255 | 20.215 | 1.00 | 0.54 | 1SG | 2132 |
| ATOM | 2132 | CE1 | PHE | 261 | 10.345 | 15.836 | 19.281 | 1.00 | 0.54 | 1SG | 2133 |
| ATOM | 2133 | CE2 | PHE | 261 | 10.393 | 17.494 | 20.982 | 1.00 | 0.54 | 1SG | 2134 |
| ATOM | 2134 | CZ | PHE | 261 | 9.942 | 16.283 | 20.515 | 1.00 | 0.54 | 1SG | 2135 |
| ATOM | 2135 | C | PHE | 261 | 14.128 | 16.739 | 18.238 | 1.00 | 0.54 | 1SG | 2136 |
| ATOM | 2136 | O | PHE | 261 | 14.251 | 15.971 | 19.192 | 1.00 | 0.54 | 1SG | 2137 |
| ATOM | 2137 | N | VAL | 262 | 14.096 | 16.302 | 16.968 | 1.00 | 0.51 | 1SG | 2138 |
| ATOM | 2138 | CA | VAL | 262 | 14.118 | 14.897 | 16.696 | 1.00 | 0.51 | 1SG | 2139 |
| ATOM | 2139 | CB | VAL | 262 | 13.875 | 14.568 | 15.253 | 1.00 | 0.51 | 1SG | 2140 |
| ATOM | 2140 | CG1 | VAL | 262 | 15.031 | 15.124 | 14.415 | 1.00 | 0.51 | 1SG | 2141 |
| ATOM | 2141 | CG2 | VAL | 262 | 13.696 | 13.046 | 15.127 | 1.00 | 0.51 | 1SG | 2142 |
| ATOM | 2142 | C | VAL | 262 | 15.437 | 14.322 | 17.098 | 1.00 | 0.51 | 1SG | 2143 |
| ATOM | 2143 | O | VAL | 262 | 15.496 | 13.244 | 17.686 | 1.00 | 0.51 | 1SG | 2144 |
| ATOM | 2144 | N | LEU | 263 | 16.539 | 15.027 | 16.795 | 1.00 | 0.52 | 1SG | 2145 |
| ATOM | 2145 | CA | LEU | 263 | 17.829 | 14.497 | 17.108 | 1.00 | 0.52 | 1SG | 2146 |
| ATOM | 2146 | CB | LEU | 263 | 19.003 | 15.305 | 16.517 | 1.00 | 0.52 | 1SG | 2147 |
| ATOM | 2147 | CG | LEU | 263 | 19.267 | 15.055 | 15.014 | 1.00 | 0.52 | 1SG | 2148 |
| ATOM | 2148 | CD2 | LEU | 263 | 20.595 | 15.694 | 14.581 | 1.00 | 0.52 | 1SG | 2149 |
| ATOM | 2149 | CD1 | LEU | 263 | 18.095 | 15.485 | 14.121 | 1.00 | 0.52 | 1SG | 2150 |
| ATOM | 2150 | C | LEU | 263 | 18.016 | 14.402 | 18.586 | 1.00 | 0.52 | 1SG | 2151 |
| ATOM | 2151 | O | LEU | 263 | 18.597 | 13.435 | 19.071 | 1.00 | 0.52 | 1SG | 2152 |
| ATOM | 2152 | N | SER | 264 | 17.578 | 15.413 | 19.359 | 1.00 | 0.56 | 1SG | 2153 |
| ATOM | 2153 | CA | SER | 264 | 17.838 | 15.305 | 20.763 | 1.00 | 0.56 | 1SG | 2154 |
| ATOM | 2154 | CB | SER | 264 | 17.510 | 16.604 | 21.518 | 1.00 | 0.56 | 1SG | 2155 |
| ATOM | 2155 | OG | SER | 264 | 18.336 | 17.660 | 21.049 | 1.00 | 0.56 | 1SG | 2156 |
| ATOM | 2156 | C | SER | 264 | 17.036 | 14.210 | 21.409 | 1.00 | 0.56 | 1SG | 2157 |
| ATOM | 2157 | O | SER | 264 | 17.587 | 13.195 | 21.832 | 1.00 | 0.56 | 1SG | 2158 |
| ATOM | 2158 | N | GLN | 265 | 15.707 | 14.422 | 21.547 | 1.00 | 0.51 | 1SG | 2159 |
| ATOM | 2159 | CA | GLN | 265 | 14.869 | 13.472 | 22.233 | 1.00 | 0.51 | 1SG | 2160 |
| ATOM | 2160 | CB | GLN | 265 | 13.832 | 14.135 | 23.159 | 1.00 | 0.51 | 1SG | 2161 |
| ATOM | 2161 | CG | GLN | 265 | 12.786 | 15.018 | 22.488 | 1.00 | 0.51 | 1SG | 2162 |
| ATOM | 2162 | CD | GLN | 265 | 11.906 | 15.550 | 23.614 | 1.00 | 0.51 | 1SG | 2163 |
| ATOM | 2163 | OE1 | GLN | 265 | 10.874 | 14.971 | 23.950 | 1.00 | 0.51 | 1SG | 2164 |
| ATOM | 2164 | NE2 | GLN | 265 | 12.341 | 16.680 | 24.234 | 1.00 | 0.51 | 1SG | 2165 |
| ATOM | 2165 | C | GLN | 265 | 14.187 | 12.418 | 21.406 | 1.00 | 0.51 | 1SG | 2166 |
| ATOM | 2166 | O | GLN | 265 | 14.122 | 11.257 | 21.798 | 1.00 | 0.51 | 1SG | 2167 |
| ATOM | 2167 | N | ALA | 266 | 13.668 | 12.780 | 20.224 | 1.00 | 0.31 | 1SG | 2168 |
| ATOM | 2168 | CA | ALA | 266 | 12.790 | 11.902 | 19.494 | 1.00 | 0.31 | 1SG | 2169 |
| ATOM | 2169 | CB | ALA | 266 | 12.273 | 12.551 | 18.200 | 1.00 | 0.31 | 1SG | 2170 |
| ATOM | 2170 | C | ALA | 266 | 13.438 | 10.605 | 19.110 | 1.00 | 0.31 | 1SG | 2171 |
| ATOM | 2171 | O | ALA | 266 | 12.828 | 9.544 | 19.225 | 1.00 | 0.31 | 1SG | 2172 |
| ATOM | 2172 | N | PHE | 267 | 14.699 | 10.624 | 18.660 | 1.00 | 0.27 | 1SG | 2173 |
| ATOM | 2173 | CA | PHE | 267 | 15.254 | 9.407 | 18.142 | 1.00 | 0.27 | 1SG | 2174 |
| ATOM | 2174 | CB | PHE | 267 | 16.695 | 9.557 | 17.612 | 1.00 | 0.27 | 1SG | 2175 |
| ATOM | 2175 | CG | PHE | 267 | 17.625 | 9.636 | 18.775 | 1.00 | 0.27 | 1SG | 2176 |
| ATOM | 2176 | CD1 | PHE | 267 | 17.852 | 10.826 | 19.421 | 1.00 | 0.27 | 1SG | 2177 |
| ATOM | 2177 | CD2 | PHE | 267 | 18.255 | 8.503 | 19.239 | 1.00 | 0.27 | 1SG | 2178 |
| ATOM | 2178 | CE1 | PHE | 267 | 18.706 | 10.890 | 20.498 | 1.00 | 0.27 | 1SG | 2179 |
| ATOM | 2179 | CE2 | PHE | 267 | 19.111 | 8.559 | 20.315 | 1.00 | 0.27 | 1SG | 2180 |
| ATOM | 2180 | CZ | PHE | 267 | 19.341 | 9.757 | 20.947 | 1.00 | 0.27 | 1SG | 2181 |
| ATOM | 2181 | C | PHE | 267 | 15.304 | 8.368 | 19.221 | 1.00 | 0.27 | 1SG | 2182 |
| ATOM | 2182 | O | PHE | 267 | 15.132 | 7.181 | 18.952 | 1.00 | 0.27 | 1SG | 2183 |
| ATOM | 2183 | N | VAL | 268 | 15.541 | 8.796 | 20.472 | 1.00 | 0.45 | 1SG | 2184 |
| ATOM | 2184 | CA | VAL | 268 | 15.777 | 7.894 | 21.561 | 1.00 | 0.45 | 1SG | 2185 |
| ATOM | 2185 | CB | VAL | 268 | 16.371 | 8.0557 | 22.764 | 1.00 | 0.45 | 1SG | 2186 |
| ATOM | 2186 | CG1 | VAL | 268 | 15.310 | 9.428 | 23.439 | 1.00 | 0.45 | 1SG | 2187 |
| ATOM | 2187 | CG2 | VAL | 268 | 16.954 | 7.458 | 23.659 | 1.00 | 0.45 | 1SG | 2188 |
| ATOM | 2188 | C | VAL | 268 | 14.573 | 7.102 | 21.992 | 1.00 | 0.45 | 1SG | 2189 |
| ATOM | 2189 | O | VAL | 268 | 14.741 | 5.991 | 22.495 | 1.00 | 0.45 | 1SG | 2190 |
| ATOM | 2190 | N | LYS | 269 | 13.335 | 7.626 | 21.859 | 1.00 | 0.56 | 1SG | 2191 |
| ATOM | 2191 | CA | LYS | 269 | 12.226 | 6.859 | 22.364 | 1.00 | 0.56 | 1SG | 2192 |
| ATOM | 2192 | CB | LYS | 269 | 10.898 | 7.636 | 22.377 | 1.00 | 0.56 | 1SG | 2193 |
| ATOM | 2193 | CG | LYS | 269 | 10.878 | 8.579 | 23.591 | 1.00 | 0.56 | 1SG | 2194 |
| ATOM | 2194 | CD | LYS | 269 | 9.880 | 9.736 | 23.541 | 1.00 | 0.56 | 1SG | 2195 |
| ATOM | 2195 | CE | LYS | 269 | 10.499 | 11.048 | 23.053 | 1.00 | 0.56 | 1SG | 2196 |
| ATOM | 2196 | NZ | LYS | 269 | 11.513 | 11.518 | 24.026 | 1.00 | 0.56 | 1SG | 2197 |
| ATOM | 2197 | C | LYS | 269 | 12.170 | 5.555 | 21.639 | 1.00 | 0.56 | 1SG | 2198 |
| ATOM | 2198 | O | LYS | 269 | 12.063 | 5.503 | 20.415 | 1.00 | 0.56 | 1SG | 2199 |
| ATOM | 2199 | N | TYR | 270 | 12.237 | 4.451 | 22.415 | 1.00 | 0.54 | 1SG | 2200 |
| ATOM | 2200 | CA | TYR | 270 | 12.449 | 3.159 | 21.831 | 1.00 | 0.54 | 1SG | 2201 |
| ATOM | 2201 | CB | TYR | 270 | 13.336 | 2.256 | 22.705 | 1.00 | 0.54 | 1SG | 2202 |
| ATOM | 2202 | CG | TYR | 270 | 13.859 | 1.152 | 21.854 | 1.00 | 0.54 | 1SG | 2203 |
| ATOM | 2203 | CD1 | TYR | 270 | 14.964 | 1.366 | 21.060 | 1.00 | 0.54 | 1SG | 2204 |
| ATOM | 2204 | CD2 | TYR | 270 | 13.264 | −0.087 | 21.845 | 1.00 | 0.54 | 1SG | 2205 |
| ATOM | 2205 | CE1 | TYR | 270 | 15.469 | 0.364 | 20.266 | 1.00 | 0.54 | 1SG | 2206 |
| ATOM | 2206 | CE2 | TYR | 270 | 13.764 | −1.093 | 21.052 | 1.00 | 0.54 | 1SG | 2207 |
| ATOM | 2207 | CZ | TYR | 270 | 14.867 | −0.869 | 20.261 | 1.00 | 0.54 | 1SG | 2208 |
| ATOM | 2208 | OH | TYR | 270 | 15.384 | −1.899 | 19.445 | 1.00 | 0.54 | 1SG | 2209 |
| ATOM | 2209 | C | TYR | 270 | 11.158 | 2.459 | 21.592 | 1.00 | 0.54 | 1SG | 2210 |
| ATOM | 2210 | O | TYR | 270 | 10.283 | 2.442 | 22.453 | 1.00 | 0.54 | 1SG | 2211 |
| ATOM | 2211 | N | ILE | 271 | 11.044 | 1.841 | 20.397 | 1.00 | 0.55 | 1SG | 2212 |
| ATOM | 2212 | CA | ILE | 271 | 9.883 | 1.127 | 19.960 | 1.00 | 0.55 | 1SG | 2213 |
| ATOM | 2213 | CB | ILE | 271 | 9.937 | 0.723 | 18.518 | 1.00 | 0.55 | 1SG | 2214 |
| ATOM | 2214 | CG2 | ILE | 271 | 11.110 | −0.258 | 18.332 | 1.00 | 0.55 | 1SG | 2215 |
| ATOM | 2215 | CG1 | ILE | 271 | 8.570 | 0.160 | 18.093 | 1.00 | 0.55 | 1SG | 2216 |
| ATOM | 2216 | CD1 | ILE | 271 | 8.405 | 0.008 | 16.584 | 1.00 | 0.55 | 1SG | 2217 |
| ATOM | 2217 | C | ILE | 271 | 9.711 | −0.113 | 20.758 | 1.00 | 0.55 | 1SG | 2218 |
| ATOM | 2218 | O | ILE | 271 | 10.683 | −0.752 | 21.156 | 1.00 | 0.55 | 1SG | 2219 |
| ATOM | 2219 | N | PHE | 272 | 8.444 | −0.497 | 21.010 | 1.00 | 0.54 | 1SG | 2220 |
| ATOM | 2220 | CA | PHE | 272 | 8.276 | −1.665 | 21.821 | 1.00 | 0.54 | 1SG | 2221 |
| ATOM | 2221 | CB | PHE | 272 | 6.915 | −1.762 | 22.521 | 1.00 | 0.54 | 1SG | 2222 |
| ATOM | 2222 | CG | PHE | 272 | 7.219 | −2.613 | 23.693 | 1.00 | 0.54 | 1SG | 2223 |
| ATOM | 2223 | CD1 | PHE | 272 | 7.283 | −3.979 | 23.584 | 1.00 | 0.54 | 1SG | 2224 |
| ATOM | 2224 | CD2 | PHE | 272 | 7.491 | −2.013 | 24.898 | 1.00 | 0.54 | 1SG | 2225 |
| ATOM | 2225 | CE1 | PHE | 272 | 7.586 | −4.743 | 24.681 | 1.00 | 0.54 | 1SG | 2226 |
| ATOM | 2226 | CE2 | PHE | 272 | 7.794 | −2.766 | 26.002 | 1.00 | 0.54 | 1SG | 2227 |
| ATOM | 2227 | CZ | PHE | 272 | 7.844 | −4.131 | 25.882 | 1.00 | 0.54 | 1SG | 2228 |
| ATOM | 2228 | C | PHE | 272 | 8.407 | −2.879 | 20.947 | 1.00 | 0.54 | 1SG | 2229 |
| ATOM | 2229 | O | PHE | 272 | 7.665 | −3.047 | 19.979 | 1.00 | 0.54 | 1SG | 2230 |
| ATOM | 2230 | N | ASN | 273 | 9.435 | −3.707 | 21.239 | 1.00 | 0.36 | 1SG | 2231 |
| ATOM | 2231 | CA | ASN | 273 | 9.718 | −4.948 | 20.563 | 1.00 | 0.36 | 1SG | 2232 |
| ATOM | 2232 | CB | ASN | 273 | 11.181 | −5.393 | 20.732 | 1.00 | 0.36 | 1SG | 2233 |
| ATOM | 2233 | CG | ASN | 273 | 12.046 | −4.509 | 19.846 | 1.00 | 0.36 | 1SG | 2234 |
| ATOM | 2234 | OD1 | ASN | 273 | 13.033 | −3.924 | 20.290 | 1.00 | 0.36 | 1SG | 2235 |
| ATOM | 2235 | ND2 | ASN | 273 | 11.664 | −4.413 | 18.544 | 1.00 | 0.36 | 1SG | 2236 |
| ATOM | 2236 | C | ASN | 273 | 8.859 | −6.091 | 21.033 | 1.00 | 0.36 | 1SG | 2237 |
| ATOM | 2237 | O | ASN | 273 | 8.395 | −6.889 | 20.220 | 1.00 | 0.36 | 1SG | 2238 |
| ATOM | 2238 | N | ASN | 274 | 8.625 | −6.209 | 22.358 | 1.00 | 0.27 | 1SG | 2239 |
| ATOM | 2239 | CA | ASN | 274 | 7.945 | −7.368 | 22.878 | 1.00 | 0.27 | 1SG | 2240 |
| ATOM | 2240 | CB | ASN | 274 | 8.202 | −7.614 | 24.377 | 1.00 | 0.27 | 1SG | 2241 |
| ATOM | 2241 | CG | ASN | 274 | 9.649 | −8.054 | 24.555 | 1.00 | 0.27 | 1SG | 2242 |
| ATOM | 2242 | OD1 | ASN | 274 | 10.111 | −8.982 | 23.895 | 1.00 | 0.27 | 1SG | 2243 |
| ATOM | 2243 | ND2 | ASN | 274 | 10.385 | −7.373 | 25.473 | 1.00 | 0.27 | 1SG | 2244 |
| ATOM | 2244 | C | ASN | 274 | 6.471 | −7.233 | 22.679 | 1.00 | 0.27 | 1SG | 2245 |
| ATOM | 2245 | O | ASN | 274 | 5.845 | −6.264 | 23.106 | 1.00 | 0.27 | 1SG | 2246 |
| ATOM | 2246 | N | SER | 275 | 5.873 | −8.245 | 22.021 | 1.00 | 0.40 | 1SG | 2247 |
| ATOM | 2247 | CA | SER | 275 | 4.469 | −8.195 | 21.744 | 1.00 | 0.40 | 1SG | 2248 |
| ATOM | 2248 | CB | SER | 275 | 4.023 | −9.230 | 20.698 | 1.00 | 0.40 | 1SG | 2249 |
| ATOM | 2249 | OG | SER | 275 | 4.650 | −8.963 | 19.453 | 1.00 | 0.40 | 1SG | 2250 |
| ATOM | 2250 | C | SER | 275 | 3.727 | −8.479 | 23.006 | 1.00 | 0.40 | 1SG | 2251 |
| ATOM | 2251 | O | SER | 275 | 4.031 | −9.435 | 23.717 | 1.00 | 0.40 | 1SG | 2252 |
| ATOM | 2252 | N | ILE | 276 | 2.730 | −7.634 | 23.327 | 1.00 | 0.50 | 1SG | 2253 |
| ATOM | 2253 | CA | ILE | 276 | 1.958 | −7.887 | 24.499 | 1.00 | 0.50 | 1SG | 2254 |
| ATOM | 2254 | CB | ILE | 276 | 2.109 | −6.838 | 25.561 | 1.00 | 0.50 | 1SG | 2255 |
| ATOM | 2255 | CG2 | ILE | 276 | 1.129 | −7.176 | 26.698 | 1.00 | 0.50 | 1SG | 2256 |
| ATOM | 2256 | CG1 | ILE | 276 | 3.575 | −6.777 | 26.027 | 1.00 | 0.50 | 1SG | 2257 |
| ATOM | 2257 | CD1 | ILE | 276 | 4.068 | −8.085 | 26.647 | 1.00 | 0.50 | 1SG | 2258 |
| ATOM | 2258 | C | ILE | 276 | 0.536 | −7.939 | 24.072 | 1.00 | 0.50 | 1SG | 2259 |
| ATOM | 2259 | O | ILE | 276 | 0.052 | −7.077 | 23.335 | 1.00 | 0.50 | 1SG | 2260 |
| ATOM | 2260 | N | VAL | 277 | −0.170 | −8.993 | 24.511 | 1.00 | 0.49 | 1SG | 2261 |
| ATOM | 2261 | CA | VAL | 277 | −1.542 | −9.093 | 24.143 | 1.00 | 0.49 | 1SG | 2262 |
| ATOM | 2262 | CB | VAL | 277 | −2.043 | −10.512 | 24.116 | 1.00 | 0.49 | 1SG | 2263 |
| ATOM | 2263 | CG1 | VAL | 277 | −1.376 | −11.225 | 22.927 | 1.00 | 0.49 | 1SG | 2264 |
| ATOM | 2264 | CG2 | VAL | 277 | −1.718 | −11.193 | 25.460 | 1.00 | 0.49 | 1SG | 2265 |
| ATOM | 2265 | C | VAL | 277 | −2.322 | −8.292 | 25.132 | 1.00 | 0.49 | 1SG | 2266 |
| ATOM | 2266 | O | VAL | 277 | −2.766 | −8.775 | 26.171 | 1.00 | 0.49 | 1SG | 2267 |
| ATOM | 2267 | N | GLN | 278 | −2.518 | −7.006 | 24.811 | 1.00 | 0.40 | 1SG | 2268 |
| ATOM | 2268 | CA | GLN | 278 | −3.303 | −6.177 | 25.667 | 1.00 | 0.40 | 1SG | 2269 |
| ATOM | 2269 | CB | GLN | 278 | −3.405 | −4.712 | 25.203 | 1.00 | 0.40 | 1SG | 2270 |
| ATOM | 2270 | CG | GLN | 278 | −2.119 | −3.902 | 25.369 | 1.00 | 0.40 | 1SG | 2271 |
| ATOM | 2271 | CD | GLN | 278 | −1.931 | −3.618 | 26.856 | 1.00 | 0.40 | 1SG | 2272 |
| ATOM | 2272 | OE1 | GLN | 278 | −2.607 | −4.189 | 27.711 | 1.00 | 0.40 | 1SG | 2273 |
| ATOM | 2273 | NE2 | GLN | 278 | −0.977 | −2.704 | 27.175 | 1.00 | 0.40 | 1SG | 2274 |
| ATOM | 2274 | C | GLN | 278 | −4.668 | −6.745 | 25.640 | 1.00 | 0.40 | 1SG | 2275 |
| ATOM | 2275 | O | GLN | 278 | −5.428 | −6.618 | 26.599 | 1.00 | 0.40 | 1SG | 2276 |
| ATOM | 2276 | N | ASP | 279 | −5.004 | −7.395 | 24.511 | 1.00 | 0.40 | 1SG | 2277 |
| ATOM | 2277 | CA | ASP | 279 | −6.311 | −7.944 | 24.350 | 1.00 | 0.40 | 1SG | 2278 |
| ATOM | 2278 | CB | ASP | 279 | −6.429 | −8.749 | 23.033 | 1.00 | 0.40 | 1SG | 2279 |
| ATOM | 2279 | CG | ASP | 279 | −6.302 | −7.762 | 21.861 | 1.00 | 0.40 | 1SG | 2280 |
| ATOM | 2280 | OD1 | ASP | 279 | −5.530 | −6.776 | 22.017 | 1.00 | 0.40 | 1SG | 2281 |
| ATOM | 2281 | OD2 | ASP | 279 | −7.017 | −7.949 | 20.839 | 1.00 | 0.40 | 1SG | 2282 |
| ATOM | 2282 | C | ASP | 279 | −6.603 | −8.848 | 25.510 | 1.00 | 0.40 | 1SG | 2283 |
| ATOM | 2283 | O | ASP | 279 | −7.669 | −8.764 | 26.114 | 1.00 | 0.40 | 1SG | 2284 |
| ATOM | 2284 | N | PHE | 280 | −5.679 | −9.741 | 25.897 | 1.00 | 0.56 | 1SG | 2285 |
| ATOM | 2285 | CA | PHE | 280 | −6.110 | −10.612 | 26.957 | 1.00 | 0.56 | 1SG | 2286 |
| ATOM | 2286 | CB | PHE | 280 | −5.623 | −12.063 | 26.871 | 1.00 | 0.56 | 1SG | 2287 |
| ATOM | 2287 | CG | PHE | 280 | −6.950 | −12.658 | 26.561 | 1.00 | 0.56 | 1SG | 2288 |
| ATOM | 2288 | CD1 | PHE | 280 | −7.764 | −12.971 | 27.629 | 1.00 | 0.56 | 1SG | 2289 |
| ATOM | 2289 | CD2 | PHE | 280 | −7.418 | −12.841 | 25.279 | 1.00 | 0.56 | 1SG | 2290 |
| ATOM | 2290 | CE1 | PHE | 280 | −9.015 | −13.507 | 27.444 | 1.00 | 0.56 | 1SG | 2291 |
| ATOM | 2291 | CE2 | PHE | 280 | −8.674 | −13.378 | 25.089 | 1.00 | 0.56 | 1SG | 2292 |
| ATOM | 2292 | CZ | PHE | 280 | −9.472 | −13.712 | 26.163 | 1.00 | 0.56 | 1SG | 2293 |
| ATOM | 2293 | C | PHE | 280 | −6.183 | −10.097 | 28.363 | 1.00 | 0.56 | 1SG | 2294 |
| ATOM | 2294 | O | PHE | 280 | −7.097 | −10.512 | 29.078 | 1.00 | 0.56 | 1SG | 2295 |
| ATOM | 2295 | N | PHE | 281 | −5.292 | −9.198 | 28.829 | 1.00 | 0.54 | 1SG | 2296 |
| ATOM | 2296 | CA | PHE | 281 | −5.508 | −8.930 | 30.219 | 1.00 | 0.54 | 1SG | 2297 |
| ATOM | 2297 | CB | PHE | 281 | −4.401 | −9.317 | 31.225 | 1.00 | 0.54 | 1SG | 2298 |
| ATOM | 2298 | CG | PHE | 281 | −3.026 | −9.025 | 30.752 | 1.00 | 0.54 | 1SG | 2299 |
| ATOM | 2299 | CD1 | PHE | 281 | −2.531 | −7.748 | 30.718 | 1.00 | 0.54 | 1SG | 2300 |
| ATOM | 2300 | CD2 | PHE | 281 | −2.216 | −10.077 | 30.387 | 1.00 | 0.54 | 1SG | 2301 |
| ATOM | 2301 | CE1 | PHE | 281 | −1.247 | −7.517 | 30.298 | 1.00 | 0.54 | 1SG | 2302 |
| ATOM | 2302 | CE2 | PHE | 281 | −0.931 | −9.864 | 29.965 | 1.00 | 0.54 | 1SG | 2303 |
| ATOM | 2303 | CZ | PHE | 281 | −0.450 | −8.573 | 29.920 | 1.00 | 0.54 | 1SG | 2304 |
| ATOM | 2304 | C | PHE | 281 | −6.130 | −7.630 | 30.575 | 1.00 | 0.54 | 1SG | 2305 |
| ATOM | 2305 | O | PHE | 281 | −6.095 | −6.649 | 29.844 | 1.00 | 0.54 | 1SG | 2306 |
| ATOM | 2306 | N | ALA | 282 | −6.736 | −7.679 | 31.777 | 1.00 | 0.47 | 1SG | 2307 |
| ATOM | 2307 | CA | ALA | 282 | −7.563 | −6.713 | 32.423 | 1.00 | 0.47 | 1SG | 2308 |
| ATOM | 2308 | CB | ALA | 282 | −7.792 | −7.012 | 33.915 | 1.00 | 0.47 | 1SG | 2309 |
| ATOM | 2309 | C | ALA | 282 | −6.973 | −5.356 | 32.357 | 1.00 | 0.47 | 1SG | 2310 |
| ATOM | 2310 | O | ALA | 282 | −5.834 | −5.130 | 32.766 | 1.00 | 0.47 | 1SG | 2311 |
| ATOM | 2311 | N | TRP | 283 | −7.778 | −4.405 | 31.840 | 1.00 | 0.50 | 1SG | 2312 |
| ATOM | 2312 | CA | TRP | 283 | −7.383 | −3.033 | 31.752 | 1.00 | 0.50 | 1SG | 2313 |
| ATOM | 2313 | CB | TRP | 283 | −8.021 | −2.261 | 30.579 | 1.00 | 0.50 | 1SG | 2314 |
| ATOM | 2314 | CG | TRP | 283 | −7.933 | −2.770 | 29.168 | 1.00 | 0.50 | 1SG | 2315 |
| ATOM | 2315 | CD2 | TRP | 283 | −6.825 | −2.550 | 28.278 | 1.00 | 0.50 | 1SG | 2316 |
| ATOM | 2316 | CD1 | TRP | 283 | −8.873 | −3.468 | 28.451 | 1.00 | 0.50 | 1SG | 2317 |
| ATOM | 2317 | NE1 | TRP | 283 | −8.432 | −3.667 | 27.169 | 1.00 | 0.50 | 1SG | 2318 |
| ATOM | 2318 | CE2 | TRP | 283 | −7.167 | −3.120 | 27.047 | 1.00 | 0.50 | 1SG | 2319 |
| ATOM | 2319 | CE3 | TRP | 283 | −5.625 | −1.930 | 28.475 | 1.00 | 0.50 | 1SG | 2320 |
| ATOM | 2320 | CZ2 | TRP | 283 | −6.301 | −3.072 | 25.995 | 1.00 | 0.50 | 1SG | 2321 |
| ATOM | 2321 | CZ3 | TRP | 283 | −4.766 | −1.885 | 27.396 | 1.00 | 0.50 | 1SG | 2322 |
| ATOM | 2322 | CH2 | TRP | 283 | −5.094 | −2.454 | 26.182 | 1.00 | 0.50 | 1SG | 2323 |
| ATOM | 2323 | C | TRP | 283 | −8.111 | −2.362 | 32.875 | 1.00 | 0.50 | 1SG | 2324 |
| ATOM | 2324 | O | TRP | 283 | −8.443 | −1.189 | 32.779 | 1.00 | 0.50 | 1SG | 2325 |
| ATOM | 2325 | N | SER | 284 | −8.384 | −3.071 | 33.975 | 1.00 | 0.46 | 1SG | 2326 |
| ATOM | 2326 | CA | SER | 284 | −9.262 | −2.560 | 34.992 | 1.00 | 0.46 | 1SG | 2327 |
| ATOM | 2327 | CB | SER | 284 | −9.510 | −3.593 | 36.115 | 1.00 | 0.46 | 1SG | 2328 |
| ATOM | 2328 | OG | SER | 284 | −8.308 | −3.845 | 36.830 | 1.00 | 0.46 | 1SG | 2329 |
| ATOM | 2329 | C | SER | 284 | −8.719 | −1.325 | 35.631 | 1.00 | 0.46 | 1SG | 2330 |
| ATOM | 2330 | O | SER | 284 | −9.464 | −0.378 | 35.886 | 1.00 | 0.46 | 1SG | 2331 |
| ATOM | 2331 | N | LYS | 285 | −7.407 | −1.289 | 35.920 | 1.00 | 0.53 | 1SG | 2332 |
| ATOM | 2332 | CA | LYS | 285 | −6.902 | −0.141 | 36.618 | 1.00 | 0.53 | 1SG | 2333 |
| ATOM | 2333 | CB | LYS | 285 | −5.519 | −0.248 | 37.295 | 1.00 | 0.53 | 1SG | 2334 |
| ATOM | 2334 | CG | LYS | 285 | −5.653 | −0.399 | 38.816 | 1.00 | 0.53 | 1SG | 2335 |
| ATOM | 2335 | CD | LYS | 285 | −4.326 | −0.422 | 39.587 | 1.00 | 0.53 | 1SG | 2336 |
| ATOM | 2336 | CE | LYS | 285 | −3.955 | −1.792 | 40.164 | 1.00 | 0.53 | 1SG | 2337 |
| ATOM | 2337 | NZ | LYS | 285 | −3.155 | −2.561 | 39.186 | 1.00 | 0.53 | 1SG | 2338 |
| ATOM | 2338 | C | LYS | 285 | −6.928 | 1.120 | 35.821 | 1.00 | 0.53 | 1SG | 2339 |
| ATOM | 2339 | O | LYS | 285 | −6.980 | 2.202 | 36.404 | 1.00 | 0.53 | 1SG | 2340 |
| ATOM | 2340 | N | ASP | 286 | −6.889 | 1.058 | 34.477 | 1.00 | 0.70 | 1SG | 2341 |
| ATOM | 2341 | CA | ASP | 286 | −6.775 | 2.311 | 33.769 | 1.00 | 0.70 | 1SG | 2342 |
| ATOM | 2342 | CB | ASP | 286 | −6.678 | 2.200 | 32.243 | 1.00 | 0.70 | 1SG | 2343 |
| ATOM | 2343 | CG | ASP | 286 | −8.071 | 2.097 | 31.629 | 1.00 | 0.70 | 1SG | 2344 |
| ATOM | 2344 | OD1 | ASP | 286 | −8.840 | 1.145 | 31.927 | 1.00 | 0.70 | 1SG | 2345 |
| ATOM | 2345 | OD2 | ASP | 286 | −8.392 | 3.018 | 30.834 | 1.00 | 0.70 | 1SG | 2346 |
| ATOM | 2346 | C | ASP | 286 | −7.986 | 3.131 | 34.021 | 1.00 | 0.70 | 1SG | 2347 |
| ATOM | 2347 | O | ASP | 286 | −7.939 | 4.357 | 33.937 | 1.00 | 0.70 | 1SG | 2348 |
| ATOM | 2348 | N | THR | 287 | −9.124 | 2.471 | 34.293 | 1.00 | 0.76 | 1SG | 2349 |
| ATOM | 2349 | CA | THR | 287 | −10.338 | 3.195 | 34.522 | 1.00 | 0.76 | 1SG | 2350 |
| ATOM | 2350 | CB | THR | 287 | −11.463 | 2.329 | 35.015 | 1.00 | 0.76 | 1SG | 2351 |
| ATOM | 2351 | OG1 | THR | 287 | −11.130 | 1.778 | 36.283 | 1.00 | 0.76 | 1SG | 2352 |
| ATOM | 2352 | CG2 | THR | 287 | −11.722 | 1.204 | 34.002 | 1.00 | 0.76 | 1SG | 2353 |
| ATOM | 2353 | C | THR | 287 | −10.105 | 4.208 | 35.591 | 1.00 | 0.76 | 1SG | 2354 |
| ATOM | 2354 | O | THR | 287 | −9.292 | 4.028 | 36.498 | 1.00 | 0.76 | 1SG | 2355 |
| ATOM | 2355 | N | TYR | 288 | −10.888 | 5.301 | 35.512 | 1.00 | 0.75 | 1SG | 2356 |
| ATOM | 2356 | CA | TYR | 288 | −11.002 | 6.411 | 36.424 | 1.00 | 0.75 | 1SG | 2357 |
| ATOM | 2357 | CB | TYR | 288 | −12.190 | 7.252 | 35.907 | 1.00 | 0.75 | 1SG | 2358 |
| ATOM | 2358 | CG | TYR | 288 | −12.372 | 8.627 | 36.447 | 1.00 | 0.75 | 1SG | 2359 |
| ATOM | 2359 | CD1 | TYR | 288 | −11.678 | 9.665 | 35.877 | 1.00 | 0.75 | 1SG | 2360 |
| ATOM | 2360 | CD2 | TYR | 288 | −13.256 | 8.891 | 37.472 | 1.00 | 0.75 | 1SG | 2361 |
| ATOM | 2361 | CE1 | TYR | 288 | −11.851 | 10.947 | 36.331 | 1.00 | 0.75 | 1SG | 2362 |
| ATOM | 2362 | CE2 | TYR | 288 | −13.433 | 10.177 | 37.932 | 1.00 | 0.75 | 1SG | 2363 |
| ATOM | 2363 | CZ | TYR | 288 | −12.727 | 11.210 | 37.357 | 1.00 | 0.75 | 1SG | 2364 |
| ATOM | 2364 | OH | TYR | 288 | −12.892 | 12.541 | 37.806 | 1.00 | 0.75 | 1SG | 2365 |
| ATOM | 2365 | C | TYR | 288 | −11.359 | 5.789 | 37.747 | 1.00 | 0.75 | 1SG | 2366 |
| ATOM | 2366 | O | TYR | 288 | −11.402 | 4.576 | 37.883 | 1.00 | 0.75 | 1SG | 2367 |
| ATOM | 2367 | N | SER | 289 | −11.619 | 6.563 | 38.801 | 1.00 | 0.82 | 1SG | 2368 |
| ATOM | 2368 | CA | SER | 289 | −11.999 | 5.892 | 40.015 | 1.00 | 0.82 | 1SG | 2369 |
| ATOM | 2369 | CB | SER | 289 | −13.366 | 5.176 | 39.903 | 1.00 | 0.82 | 1SG | 2370 |
| ATOM | 2370 | OG | SER | 289 | −14.419 | 6.110 | 39.697 | 1.00 | 0.82 | 1SG | 2371 |
| ATOM | 2371 | C | SER | 289 | −10.866 | 5.000 | 40.526 | 1.00 | 0.82 | 1SG | 2372 |
| ATOM | 2372 | O | SER | 289 | −10.167 | 5.513 | 41.394 | 1.00 | 0.82 | 1SG | 2373 |
| ATOM | 2373 | N | PRO | 290 | −10.589 | 3.746 | 40.149 | 1.00 | 0.70 | 1SG | 2374 |
| ATOM | 2374 | CA | PRO | 290 | −9.417 | 3.103 | 40.687 | 1.00 | 0.70 | 1SG | 2375 |
| ATOM | 2375 | CD | PRO | 290 | −11.645 | 2.745 | 39.980 | 1.00 | 0.70 | 1SG | 2376 |
| ATOM | 2376 | CB | PRO | 290 | −9.555 | 1.620 | 40.367 | 1.00 | 0.70 | 1SG | 2377 |
| ATOM | 2377 | CG | PRO | 290 | −11.066 | 1.390 | 40.382 | 1.00 | 0.70 | 1SG | 2378 |
| ATOM | 2378 | C | PRO | 290 | −8.058 | 3.639 | 40.346 | 1.00 | 0.70 | 1SG | 2379 |
| ATOM | 2379 | O | PRO | 290 | −7.164 | 3.509 | 41.181 | 1.00 | 0.70 | 1SG | 2380 |
| ATOM | 2380 | N | ASP | 291 | −7.832 | 4.203 | 39.149 | 1.00 | 0.49 | 1SG | 2381 |
| ATOM | 2381 | CA | ASP | 291 | −6.492 | 4.648 | 38.902 | 1.00 | 0.49 | 1SG | 2382 |
| ATOM | 2382 | CB | ASP | 291 | −6.275 | 5.254 | 37.503 | 1.00 | 0.49 | 1SG | 2383 |
| ATOM | 2383 | CG | ASP | 291 | −4.781 | 5.467 | 37.312 | 1.00 | 0.49 | 1SG | 2384 |
| ATOM | 2384 | OD1 | ASP | 291 | −4.008 | 4.897 | 38.125 | 1.00 | 0.49 | 1SG | 2385 |
| ATOM | 2385 | OD2 | ASP | 291 | −4.388 | 6.192 | 36.358 | 1.00 | 0.49 | 1SG | 2386 |
| ATOM | 2386 | C | ASP | 291 | −6.210 | 5.726 | 39.886 | 1.00 | 0.49 | 1SG | 2387 |
| ATOM | 2387 | O | ASP | 291 | −5.147 | 5.761 | 40.502 | 1.00 | 0.49 | 1SG | 2388 |
| ATOM | 2388 | N | GLU | 292 | −7.194 | 6.616 | 40.089 | 1.00 | 0.54 | 1SG | 2389 |
| ATOM | 2389 | CA | GLU | 292 | −6.979 | 7.730 | 40.961 | 1.00 | 0.54 | 1SG | 2390 |
| ATOM | 2390 | CB | GLU | 292 | −8.154 | 8.725 | 40.966 | 1.00 | 0.54 | 1SG | 2391 |
| ATOM | 2391 | CG | GLU | 292 | −9.387 | 8.283 | 41.756 | 1.00 | 0.54 | 1SG | 2392 |
| ATOM | 2392 | CD | GLU | 292 | −9.233 | 8.844 | 43.164 | 1.00 | 0.54 | 1SG | 2393 |
| ATOM | 2393 | OE1 | GLU | 292 | −8.951 | 10.067 | 43.279 | 1.00 | 0.54 | 1SG | 2394 |
| ATOM | 2394 | OE2 | GLU | 292 | −9.388 | 8.061 | 44.142 | 1.00 | 0.54 | 1SG | 2395 |
| ATOM | 2395 | C | GLU | 292 | −6.761 | 7.230 | 42.358 | 1.00 | 0.54 | 1SG | 2396 |
| ATOM | 2396 | O | GLU | 292 | −6.003 | 7.835 | 43.111 | 1.00 | 0.54 | 1SG | 2397 |
| ATOM | 2397 | N | HIS | 293 | −7.466 | 6.158 | 42.774 | 1.00 | 0.58 | 1SG | 2398 |
| ATOM | 2398 | CA | HIS | 293 | −7.299 | 5.655 | 44.113 | 1.00 | 0.58 | 1SG | 2399 |
| ATOM | 2399 | ND1 | HIS | 293 | −8.643 | 4.483 | 46.969 | 1.00 | 0.58 | 1SG | 2400 |
| ATOM | 2400 | CG | HIS | 293 | −8.153 | 3.945 | 45.801 | 1.00 | 0.58 | 1SG | 2401 |
| ATOM | 2401 | CB | HIS | 293 | −8.332 | 4.563 | 44.445 | 1.00 | 0.58 | 1SG | 2402 |
| ATOM | 2402 | NE2 | HIS | 293 | −7.594 | 2.603 | 47.528 | 1.00 | 0.58 | 1SG | 2403 |
| ATOM | 2403 | CD2 | HIS | 293 | −7.514 | 2.796 | 46.160 | 1.00 | 0.58 | 1SG | 2404 |
| ATOM | 2404 | CE1 | HIS | 293 | −8.281 | 3.642 | 47.970 | 1.00 | 0.58 | 1SG | 2405 |
| ATOM | 2405 | C | HIS | 293 | −5.952 | 5.029 | 44.339 | 1.00 | 0.58 | 1SG | 2406 |
| ATOM | 2406 | O | HIS | 293 | −5.151 | 5.523 | 45.132 | 1.00 | 0.58 | 1SG | 2407 |
| ATOM | 2407 | N | PHE | 294 | −5.661 | 3.919 | 43.626 | 1.00 | 0.48 | 1SG | 2408 |
| ATOM | 2408 | CA | PHE | 294 | −4.447 | 3.200 | 43.903 | 1.00 | 0.48 | 1SG | 2409 |
| ATOM | 2409 | CB | PHE | 294 | −4.432 | 1.732 | 43.433 | 1.00 | 0.48 | 1SG | 2410 |
| ATOM | 2410 | CG | PHE | 294 | −5.142 | 0.914 | 44.464 | 1.00 | 0.48 | 1SG | 2411 |
| ATOM | 2411 | CD1 | PHE | 294 | −4.454 | 0.437 | 45.559 | 1.00 | 0.48 | 1SG | 2412 |
| ATOM | 2412 | CD2 | PHE | 294 | −6.485 | 0.631 | 44.357 | 1.00 | 0.48 | 1SG | 2413 |
| ATOM | 2413 | CE1 | PHE | 294 | −5.084 | −0.317 | 46.522 | 1.00 | 0.48 | 1SG | 2414 |
| ATOM | 2414 | CE2 | PHE | 294 | −7.122 | −0.124 | 45.317 | 1.00 | 0.48 | 1SG | 2415 |
| ATOM | 2415 | CZ | PHE | 294 | −6.423 | −0.601 | 46.401 | 1.00 | 0.48 | 1SG | 2416 |
| ATOM | 2416 | C | PHE | 294 | −3.209 | 3.863 | 43.395 | 1.00 | 0.48 | 1SG | 2417 |
| ATOM | 2417 | O | PHE | 294 | −2.213 | 3.943 | 44.110 | 1.00 | 0.48 | 1SG | 2418 |
| ATOM | 2418 | N | TRP | 295 | −3.230 | 4.374 | 42.156 | 1.00 | 0.28 | 1SG | 2419 |
| ATOM | 2419 | CA | TRP | 295 | −2.003 | 4.859 | 41.600 | 1.00 | 0.28 | 1SG | 2420 |
| ATOM | 2420 | CB | TRP | 295 | −2.191 | 5.265 | 40.134 | 1.00 | 0.28 | 1SG | 2421 |
| ATOM | 2421 | CG | TRP | 295 | −0.922 | 5.301 | 39.329 | 1.00 | 0.28 | 1SG | 2422 |
| ATOM | 2422 | CD2 | TRP | 295 | −0.022 | 6.411 | 39.311 | 1.00 | 0.28 | 1SG | 2423 |
| ATOM | 2423 | CD1 | TRP | 295 | −0.386 | 4.347 | 38.513 | 1.00 | 0.28 | 1SG | 2424 |
| ATOM | 2424 | NE1 | TRP | 295 | 0.801 | 4.796 | 37.987 | 1.00 | 0.28 | 1SG | 2425 |
| ATOM | 2425 | CE2 | TRP | 295 | 1.035 | 6.066 | 38.471 | 1.00 | 0.28 | 1SG | 2426 |
| ATOM | 2426 | CE3 | TRP | 295 | −0.076 | 7.615 | 39.947 | 1.00 | 0.28 | 1SG | 2427 |
| ATOM | 2427 | CZ2 | TRP | 295 | 2.064 | 6.932 | 38.252 | 1.00 | 0.28 | 1SG | 2428 |
| ATOM | 2428 | CZ3 | TRP | 295 | 0.962 | 8.487 | 39.728 | 1.00 | 0.28 | 1SG | 2429 |
| ATOM | 2429 | CH2 | TRP | 295 | 2.003 | 8.146 | 38.896 | 1.00 | 0.28 | 1SG | 2430 |
| ATOM | 2430 | C | TRP | 295 | −1.512 | 6.030 | 42.404 | 1.00 | 0.28 | 1SG | 2431 |
| ATOM | 2431 | O | TRP | 295 | −0.340 | 6.095 | 42.772 | 1.00 | 0.28 | 1SG | 2432 |
| ATOM | 2432 | N | ALA | 296 | −2.414 | 6.976 | 42.727 | 1.00 | 0.24 | 1SG | 2433 |
| ATOM | 2433 | CA | ALA | 296 | −2.058 | 8.167 | 43.451 | 1.00 | 0.24 | 1SG | 2434 |
| ATOM | 2434 | CB | ALA | 296 | −3.236 | 9.143 | 43.600 | 1.00 | 0.24 | 1SG | 2435 |
| ATOM | 2435 | C | ALA | 296 | −1.597 | 7.809 | 44.830 | 1.00 | 0.24 | 1SG | 2436 |
| ATOM | 2436 | O | ALA | 296 | −0.644 | 8.392 | 45.344 | 1.00 | 0.24 | 1SG | 2437 |
| ATOM | 2437 | N | THR | 297 | −2.259 | 6.843 | 45.488 | 1.00 | 0.43 | 1SG | 2438 |
| ATOM | 2438 | CA | THR | 297 | −1.819 | 6.524 | 46.815 | 1.00 | 0.43 | 1SG | 2439 |
| ATOM | 2439 | CB | THR | 297 | −2.651 | 5.475 | 47.506 | 1.00 | 0.43 | 1SG | 2440 |
| ATOM | 2440 | OG1 | THR | 297 | −2.303 | 5.409 | 48.882 | 1.00 | 0.43 | 1SG | 2441 |
| ATOM | 2441 | CG2 | THR | 297 | −2.405 | 4.111 | 46.841 | 1.00 | 0.43 | 1SG | 2442 |
| ATOM | 2442 | C | THR | 297 | −0.428 | 6.007 | 46.687 | 1.00 | 0.43 | 1SG | 2443 |
| ATOM | 2443 | O | THR | 297 | 0.427 | 6.244 | 47.538 | 1.00 | 0.43 | 1SG | 2444 |
| ATOM | 2444 | N | LEU | 298 | −0.173 | 5.299 | 45.574 | 1.00 | 0.52 | 1SG | 2445 |
| ATOM | 2445 | CA | LEU | 298 | 1.085 | 4.676 | 45.318 | 1.00 | 0.52 | 1SG | 2446 |
| ATOM | 2446 | CB | LEU | 298 | 1.073 | 3.910 | 43.975 | 1.00 | 0.52 | 1SG | 2447 |
| ATOM | 2447 | CG | LEU | 298 | 2.273 | 2.973 | 43.689 | 1.00 | 0.52 | 1SG | 2448 |
| ATOM | 2448 | CD2 | LEU | 298 | 3.638 | 3.671 | 43.803 | 1.00 | 0.52 | 1SG | 2449 |
| ATOM | 2449 | CD1 | LEU | 298 | 2.105 | 2.293 | 42.320 | 1.00 | 0.52 | 1SG | 2450 |
| ATOM | 2450 | C | LEU | 298 | 2.146 | 5.732 | 45.269 | 1.00 | 0.52 | 1SG | 2451 |
| ATOM | 2451 | O | LEU | 298 | 3.220 | 5.495 | 45.803 | 1.00 | 0.52 | 1SG | 2452 |
| ATOM | 2452 | N | ILE | 299 | 1.906 | 6.880 | 44.597 | 1.00 | 0.50 | 1SG | 2453 |
| ATOM | 2453 | CA | ILE | 299 | 2.867 | 7.961 | 44.500 | 1.00 | 0.50 | 1SG | 2454 |
| ATOM | 2454 | CB | ILE | 299 | 2.566 | 8.989 | 43.454 | 1.00 | 0.50 | 1SG | 2455 |
| ATOM | 2455 | CG2 | ILE | 299 | 1.192 | 9.618 | 43.726 | 1.00 | 0.50 | 1SG | 2456 |
| ATOM | 2456 | CG1 | ILE | 299 | 3.709 | 10.014 | 43.455 | 1.00 | 0.50 | 1SG | 2457 |
| ATOM | 2457 | CD1 | ILE | 299 | 3.550 | 11.129 | 42.433 | 1.00 | 0.50 | 1SG | 2458 |
| ATOM | 2458 | C | ILE | 299 | 2.999 | 8.745 | 45.774 | 1.00 | 0.50 | 1SG | 2459 |
| ATOM | 2459 | O | ILE | 299 | 4.057 | 9.297 | 46.075 | 1.00 | 0.50 | 1SG | 2460 |
| ATOM | 2460 | N | ARG | 300 | 1.887 | 8.923 | 46.506 | 1.00 | 0.48 | 1SG | 2461 |
| ATOM | 2461 | CA | ARG | 300 | 1.904 | 9.686 | 47.724 | 1.00 | 0.48 | 1SG | 2462 |
| ATOM | 2462 | CB | ARG | 300 | 0.491 | 9.967 | 48.257 | 1.00 | 0.48 | 1SG | 2463 |
| ATOM | 2463 | CG | ARG | 300 | −0.400 | 10.742 | 47.288 | 1.00 | 0.48 | 1SG | 2464 |
| ATOM | 2464 | CD | ARG | 300 | −1.824 | 10.933 | 47.810 | 1.00 | 0.48 | 1SG | 2465 |
| ATOM | 2465 | NE | ARG | 300 | −2.590 | 11.683 | 46.777 | 1.00 | 0.48 | 1SG | 2466 |
| ATOM | 2466 | CZ | ARG | 300 | −3.913 | 11.406 | 46.589 | 1.00 | 0.48 | 1SG | 2467 |
| ATOM | 2467 | NH1 | ARG | 300 | −4.517 | 10.439 | 47.339 | 1.00 | 0.48 | 1SG | 2468 |
| ATOM | 2468 | NH2 | ARG | 300 | −4.630 | 12.094 | 45.654 | 1.00 | 0.48 | 1SG | 2469 |
| ATOM | 2469 | C | ARG | 300 | 2.603 | 8.916 | 48.801 | 1.00 | 0.48 | 1SG | 2470 |
| ATOM | 2470 | O | ARG | 300 | 3.267 | 9.493 | 49.659 | 1.00 | 0.48 | 1SG | 2471 |
| ATOM | 2471 | N | VAL | 301 | 2.417 | 7.584 | 48.814 | 1.00 | 0.44 | 1SG | 2472 |
| ATOM | 2472 | CA | VAL | 301 | 2.999 | 6.758 | 49.836 | 1.00 | 0.44 | 1SG | 2473 |
| ATOM | 2473 | CB | VAL | 301 | 2.533 | 5.332 | 49.777 | 1.00 | 0.44 | 1SG | 2474 |
| ATOM | 2474 | CG1 | VAL | 301 | 1.018 | 5.301 | 50.046 | 1.00 | 0.44 | 1SG | 2475 |
| ATOM | 2475 | CG2 | VAL | 301 | 2.950 | 4.732 | 48.425 | 1.00 | 0.44 | 1SG | 2476 |
| ATOM | 2476 | C | VAL | 301 | 4.513 | 6.734 | 49.824 | 1.00 | 0.44 | 1SG | 2477 |
| ATOM | 2477 | O | VAL | 301 | 5.086 | 6.920 | 50.896 | 1.00 | 0.44 | 1SG | 2478 |
| ATOM | 2478 | N | PRO | 302 | 5.225 | 6.534 | 48.729 | 1.00 | 0.34 | 1SG | 2479 |
| ATOM | 2479 | CA | PRO | 302 | 6.645 | 6.372 | 48.778 | 1.00 | 0.34 | 1SG | 2480 |
| ATOM | 2480 | CD | PRO | 302 | 4.858 | 7.020 | 47.432 | 1.00 | 0.34 | 1SG | 2481 |
| ATOM | 2481 | CB | PRO | 302 | 7.122 | 6.248 | 47.338 | 1.00 | 0.34 | 1SG | 2482 |
| ATOM | 2482 | CG | PRO | 302 | 6.145 | 7.168 | 46.610 | 1.00 | 0.34 | 1SG | 2483 |
| ATOM | 2483 | C | PRO | 302 | 7.307 | 7.544 | 49.413 | 1.00 | 0.34 | 1SG | 2484 |
| ATOM | 2484 | O | PRO | 302 | 6.725 | 8.627 | 49.453 | 1.00 | 0.34 | 1SG | 2485 |
| ATOM | 2485 | N | GLY | 303 | 8.539 | 7.331 | 49.907 | 1.00 | 0.34 | 1SG | 2486 |
| ATOM | 2486 | CA | GLY | 303 | 9.268 | 8.364 | 50.574 | 1.00 | 0.34 | 1SG | 2487 |
| ATOM | 2487 | C | GLY | 303 | 9.704 | 9.375 | 49.566 | 1.00 | 0.34 | 1SG | 2488 |
| ATOM | 2488 | O | GLY | 303 | 9.720 | 9.117 | 48.363 | 1.00 | 0.34 | 1SG | 2489 |
| ATOM | 2489 | N | ILE | 304 | 10.082 | 10.567 | 50.070 | 1.00 | 0.46 | 1SG | 2490 |
| ATOM | 2490 | CA | ILE | 304 | 10.506 | 11.673 | 49.260 | 1.00 | 0.46 | 1SG | 2491 |
| ATOM | 2491 | CB | ILE | 304 | 10.109 | 13.004 | 49.843 | 1.00 | 0.46 | 1SG | 2492 |
| ATOM | 2492 | CG2 | ILE | 304 | 10.714 | 14.090 | 48.939 | 1.00 | 0.46 | 1SG | 2493 |
| ATOM | 2493 | CG1 | ILE | 304 | 8.592 | 13.155 | 50.047 | 1.00 | 0.46 | 1SG | 2494 |
| ATOM | 2494 | CD1 | ILE | 304 | 7.794 | 13.319 | 48.759 | 1.00 | 0.46 | 1SG | 2495 |
| ATOM | 2495 | C | ILE | 304 | 12.003 | 11.715 | 49.324 | 1.00 | 0.46 | 1SG | 2496 |
| ATOM | 2496 | O | ILE | 304 | 12.554 | 11.896 | 50.408 | 1.00 | 0.46 | 1SG | 2497 |
| ATOM | 2497 | N | PRO | 305 | 12.700 | 11.507 | 48.239 | 1.00 | 0.39 | 1SG | 2498 |
| ATOM | 2498 | CA | PRO | 305 | 14.126 | 11.686 | 48.310 | 1.00 | 0.39 | 1SG | 2499 |
| ATOM | 2499 | CD | PRO | 305 | 12.363 | 10.396 | 47.366 | 1.00 | 0.39 | 1SG | 2500 |
| ATOM | 2500 | CB | PRO | 305 | 14.764 | 10.536 | 47.544 | 1.00 | 0.39 | 1SG | 2501 |
| ATOM | 2501 | CG | PRO | 305 | 13.656 | 10.104 | 46.586 | 1.00 | 0.39 | 1SG | 2502 |
| ATOM | 2502 | C | PRO | 305 | 14.504 | 12.999 | 47.708 | 1.00 | 0.39 | 1SG | 2503 |
| ATOM | 2503 | O | PRO | 305 | 13.743 | 13.524 | 46.897 | 1.00 | 0.39 | 1SG | 2504 |
| ATOM | 2504 | N | GLY | 306 | 15.692 | 13.530 | 48.046 | 1.00 | 0.43 | 1SG | 2505 |
| ATOM | 2505 | CA | GLY | 306 | 16.109 | 14.745 | 47.415 | 1.00 | 0.43 | 1SG | 2506 |
| ATOM | 2506 | C | GLY | 306 | 16.556 | 14.350 | 46.046 | 1.00 | 0.43 | 1SG | 2507 |
| ATOM | 2507 | O | GLY | 306 | 17.009 | 13.226 | 45.835 | 1.00 | 0.43 | 1SG | 2508 |
| ATOM | 2508 | N | GLU | 307 | 16.466 | 15.275 | 45.072 | 1.00 | 0.77 | 1SG | 2509 |
| ATOM | 2509 | CA | GLU | 307 | 16.867 | 14.922 | 43.741 | 1.00 | 0.77 | 1SG | 2510 |
| ATOM | 2510 | CB | GLU | 307 | 16.819 | 16.076 | 42.730 | 1.00 | 0.77 | 1SG | 2511 |
| ATOM | 2511 | CG | GLU | 307 | 17.875 | 15.926 | 41.620 | 1.00 | 0.77 | 1SG | 2512 |
| ATOM | 2512 | CD | GLU | 307 | 17.702 | 14.606 | 40.870 | 1.00 | 0.77 | 1SG | 2513 |
| ATOM | 2513 | OE1 | GLU | 307 | 16.757 | 13.848 | 41.215 | 1.00 | 0.77 | 1SG | 2514 |
| ATOM | 2514 | OE2 | GLU | 307 | 18.513 | 14.343 | 39.943 | 1.00 | 0.77 | 1SG | 2515 |
| ATOM | 2515 | C | GLU | 307 | 18.290 | 14.486 | 43.758 | 1.00 | 0.77 | 1SG | 2516 |
| ATOM | 2516 | O | GLU | 307 | 18.637 | 13.471 | 43.158 | 1.00 | 0.77 | 1SG | 2517 |
| ATOM | 2517 | N | ILE | 308 | 19.145 | 15.217 | 44.491 | 1.00 | 0.73 | 1SG | 2518 |
| ATOM | 2518 | CA | ILE | 308 | 20.552 | 14.952 | 44.463 | 1.00 | 0.73 | 1SG | 2519 |
| ATOM | 2519 | CB | ILE | 308 | 21.333 | 15.812 | 45.417 | 1.00 | 0.73 | 1SG | 2520 |
| ATOM | 2520 | CG2 | ILE | 308 | 21.129 | 17.277 | 44.996 | 1.00 | 0.73 | 1SG | 2521 |
| ATOM | 2521 | CG1 | ILE | 308 | 20.938 | 15.528 | 46.879 | 1.00 | 0.73 | 1SG | 2522 |
| ATOM | 2522 | CD1 | ILE | 308 | 19.486 | 15.865 | 47.217 | 1.00 | 0.73 | 1SG | 2523 |
| ATOM | 2523 | C | ILE | 308 | 20.777 | 13.532 | 44.862 | 1.00 | 0.73 | 1SG | 2524 |
| ATOM | 2524 | O | ILE | 308 | 21.632 | 12.848 | 44.302 | 1.00 | 0.73 | 1SG | 2525 |
| ATOM | 2525 | N | SER | 309 | 19.997 | 13.048 | 45.840 | 1.00 | 0.54 | 1SG | 2526 |
| ATOM | 2526 | CA | SER | 309 | 20.148 | 11.707 | 46.313 | 1.00 | 0.54 | 1SG | 2527 |
| ATOM | 2527 | CB | SER | 309 | 19.149 | 11.397 | 47.441 | 1.00 | 0.54 | 1SG | 2528 |
| ATOM | 2528 | OG | SER | 309 | 19.415 | 12.218 | 48.568 | 1.00 | 0.54 | 1SG | 2529 |
| ATOM | 2529 | C | SER | 309 | 19.874 | 10.758 | 45.183 | 1.00 | 0.54 | 1SG | 2530 |
| ATOM | 2530 | O | SER | 309 | 20.613 | 9.796 | 44.980 | 1.00 | 0.54 | 1SG | 2531 |
| ATOM | 2531 | N | ARG | 310 | 18.799 | 11.011 | 44.411 | 1.00 | 0.63 | 1SG | 2532 |
| ATOM | 2532 | CA | ARG | 310 | 18.403 | 10.121 | 43.356 | 1.00 | 0.63 | 1SG | 2533 |
| ATOM | 2533 | CB | ARG | 310 | 17.064 | 10.509 | 42.714 | 1.00 | 0.63 | 1SG | 2534 |
| ATOM | 2534 | CG | ARG | 310 | 16.630 | 9.537 | 41.617 | 1.00 | 0.63 | 1SG | 2535 |
| ATOM | 2535 | CD | ARG | 310 | 15.295 | 9.903 | 40.978 | 1.00 | 0.63 | 1SG | 2536 |
| ATOM | 2536 | NE | ARG | 310 | 14.286 | 9.948 | 42.073 | 1.00 | 0.63 | 1SG | 2537 |
| ATOM | 2537 | CZ | ARG | 310 | 14.031 | 11.124 | 42.715 | 1.00 | 0.63 | 1SG | 2538 |
| ATOM | 2538 | NH1 | ARG | 310 | 14.688 | 12.261 | 42.341 | 1.00 | 0.63 | 1SG | 2539 |
| ATOM | 2539 | NH2 | ARG | 310 | 13.113 | 11.165 | 43.724 | 100 | 0.63 | 1SG | 2540 |
| ATOM | 2540 | C | ARG | 310 | 19.426 | 10.091 | 42.260 | 1.00 | 0.63 | 1SG | 2541 |
| ATOM | 2541 | O | ARG | 310 | 19.784 | 9.019 | 41.773 | 1.00 | 0.63 | 1SG | 2542 |
| ATOM | 2542 | N | SER | 311 | 19.932 | 11.268 | 41.848 | 1.00 | 0.51 | 1SG | 2543 |
| ATOM | 2543 | CA | SER | 311 | 20.854 | 11.342 | 40.747 | 1.00 | 0.51 | 1SG | 2544 |
| ATOM | 2544 | CB | SER | 311 | 21.263 | 12.784 | 40.410 | 1.00 | 0.51 | 1SG | 2545 |
| ATOM | 2545 | OG | SER | 311 | 22.177 | 12.792 | 39.322 | 1.00 | 0.51 | 1SG | 2546 |
| ATOM | 2546 | C | SER | 311 | 22.106 | 10.599 | 41.110 | 1.00 | 0.51 | 1SG | 2547 |
| ATOM | 2547 | O | SER | 311 | 22.669 | 9.869 | 40.295 | 1.00 | 0.51 | 1SG | 2548 |
| ATOM | 2548 | N | ALA | 312 | 22.563 | 10.779 | 42.361 | 1.00 | 0.37 | 1SG | 2549 |
| ATOM | 2549 | CA | ALA | 312 | 23.756 | 10.191 | 42.898 | 1.00 | 0.37 | 1SG | 2550 |
| ATOM | 2550 | CB | ALA | 312 | 24.112 | 10.735 | 44.292 | 1.00 | 0.37 | 1SG | 2551 |
| ATOM | 2551 | C | ALA | 312 | 23.566 | 8.716 | 43.025 | 1.00 | 0.37 | 1SG | 2552 |
| ATOM | 2552 | O | ALA | 312 | 24.542 | 7.971 | 43.059 | 1.00 | 0.37 | 1SG | 2553 |
| ATOM | 2553 | N | GLN | 313 | 22.293 | 8.283 | 43.134 | 1.00 | 0.51 | 1SG | 2554 |
| ATOM | 2554 | CA | GLN | 313 | 21.856 | 6.929 | 43.336 | 1.00 | 0.51 | 1SG | 2555 |
| ATOM | 2555 | CB | GLN | 313 | 22.276 | 5.881 | 42.273 | 1.00 | 0.51 | 1SG | 2556 |
| ATOM | 2556 | CG | GLN | 313 | 23.756 | 5.514 | 42.185 | 1.00 | 0.51 | 1SG | 2557 |
| ATOM | 2557 | CD | GLN | 313 | 23.902 | 4.481 | 41.075 | 1.00 | 0.51 | 1SG | 2558 |
| ATOM | 2558 | OE1 | GLN | 313 | 22.911 | 3.977 | 40.550 | 1.00 | 0.51 | 1SG | 2559 |
| ATOM | 2559 | NE2 | GLN | 313 | 25.170 | 4.153 | 40.711 | 1.00 | 0.51 | 1SG | 2560 |
| ATOM | 2560 | C | GLN | 313 | 22.250 | 6.483 | 44.702 | 1.00 | 0.51 | 1SG | 2561 |
| ATOM | 2561 | O | GLN | 313 | 22.790 | 5.395 | 44.896 | 1.00 | 0.51 | 1SG | 2562 |
| ATOM | 2562 | N | ASP | 314 | 21.989 | 7.346 | 45.701 | 1.00 | 0.60 | 1SG | 2563 |
| ATOM | 2563 | CA | ASP | 314 | 22.228 | 6.941 | 47.053 | 1.00 | 0.60 | 1SG | 2564 |
| ATOM | 2564 | CB | ASP | 314 | 22.592 | 8.082 | 48.028 | 1.00 | 0.60 | 1SG | 2565 |
| ATOM | 2565 | CG | ASP | 314 | 21.424 | 9.046 | 48.201 | 1.00 | 0.60 | 1SG | 2566 |
| ATOM | 2566 | OD1 | ASP | 314 | 20.468 | 9.000 | 47.382 | 1.00 | 0.60 | 1SG | 2567 |
| ATOM | 2567 | OD2 | ASP | 314 | 21.479 | 9.853 | 49.167 | 1.00 | 0.60 | 1SG | 2568 |
| ATOM | 2568 | C | ASP | 314 | 20.947 | 6.319 | 47.507 | 1.00 | 0.60 | 1SG | 2569 |
| ATOM | 2569 | O | ASP | 314 | 19.954 | 6.371 | 46.784 | 1.00 | 0.60 | 1SG | 2570 |
| ATOM | 2570 | N | VAL | 315 | 20.951 | 5.727 | 48.717 | 1.00 | 0.51 | 1SG | 2571 |
| ATOM | 2571 | CA | VAL | 315 | 19.809 | 5.074 | 49.299 | 1.00 | 0.51 | 1SG | 2572 |
| ATOM | 2572 | CB | VAL | 315 | 18.810 | 6.016 | 49.920 | 1.00 | 0.51 | 1SG | 2573 |
| ATOM | 2573 | CG1 | VAL | 315 | 19.493 | 6.728 | 51.099 | 1.00 | 0.51 | 1SG | 2574 |
| ATOM | 2574 | CG2 | VAL | 315 | 18.262 | 6.977 | 48.852 | 1.00 | 0.51 | 1SG | 2575 |
| ATOM | 2575 | C | VAL | 315 | 19.136 | 4.225 | 48.266 | 1.00 | 0.51 | 1SG | 2576 |
| ATOM | 2576 | O | VAL | 315 | 17.960 | 4.403 | 47.952 | 1.00 | 0.51 | 1SG | 2577 |
| ATOM | 2577 | N | SER | 316 | 19.889 | 3.241 | 47.743 | 1.00 | 0.36 | 1SG | 2578 |
| ATOM | 2578 | CA | SER | 316 | 19.447 | 2.348 | 46.712 | 1.00 | 0.36 | 1SG | 2579 |
| ATOM | 2579 | CB | SER | 316 | 20.540 | 1.359 | 46.274 | 1.00 | 0.36 | 1SG | 2580 |
| ATOM | 2580 | OG | SER | 316 | 21.626 | 2.059 | 45.685 | 1.00 | 0.36 | 1SG | 2581 |
| ATOM | 2581 | C | SER | 316 | 18.298 | 1.546 | 47.226 | 1.00 | 0.36 | 1SG | 2582 |
| ATOM | 2582 | O | SER | 316 | 17.455 | 1.087 | 46.457 | 1.00 | 0.36 | 1SG | 2583 |
| ATOM | 2583 | N | ASP | 317 | 18.232 | 1.362 | 48.553 | 1.00 | 0.39 | 1SG | 2584 |
| ATOM | 2584 | CA | ASP | 317 | 17.221 | 0.535 | 49.139 | 1.00 | 0.39 | 1SG | 2585 |
| ATOM | 2585 | CB | ASP | 317 | 17.253 | 0.565 | 50.674 | 1.00 | 0.39 | 1SG | 2586 |
| ATOM | 2586 | CG | ASP | 317 | 16.355 | −0.551 | 51.178 | 1.00 | 0.39 | 1SG | 2587 |
| ATOM | 2587 | OD1 | ASP | 317 | 15.778 | −1.281 | 50.329 | 1.00 | 0.39 | 1SG | 2588 |
| ATOM | 2588 | OD2 | ASP | 317 | 16.233 | −0.692 | 52.424 | 1.00 | 0.39 | 1SG | 2589 |
| ATOM | 2589 | C | ASP | 317 | 15.878 | 1.032 | 48.699 | 1.00 | 0.39 | 1SG | 2590 |
| ATOM | 2590 | O | ASP | 317 | 14.971 | 0.236 | 48.461 | 1.00 | 0.39 | 1SG | 2591 |
| ATOM | 2591 | N | LEU | 318 | 15.706 | 2.361 | 48.569 | 1.00 | 0.55 | 1SG | 2592 |
| ATOM | 2592 | CA | LEU | 318 | 14.416 | 2.859 | 48.184 | 1.00 | 0.55 | 1SG | 2593 |
| ATOM | 2593 | CB | LEU | 318 | 14.236 | 4.357 | 48.472 | 1.00 | 0.55 | 1SG | 2594 |
| ATOM | 2594 | CG | LEU | 318 | 14.327 | 4.700 | 49.968 | 1.00 | 0.55 | 1SG | 2595 |
| ATOM | 2595 | CD2 | LEU | 318 | 13.840 | 6.129 | 50.240 | 1.00 | 0.55 | 1SG | 2596 |
| ATOM | 2596 | CD1 | LEU | 318 | 15.737 | 4.436 | 50.519 | 1.00 | 0.55 | 1SG | 2597 |
| ATOM | 2597 | C | LEU | 318 | 14.227 | 2.649 | 46.712 | 1.00 | 0.55 | 1SG | 2598 |
| ATOM | 2598 | O | LEU | 318 | 14.671 | 3.444 | 45.885 | 1.00 | 0.55 | 1SG | 2599 |
| ATOM | 2599 | N | GLN | 319 | 13.527 | 1.555 | 46.358 | 1.00 | 0.63 | 1SG | 2600 |
| ATOM | 2600 | CA | GLN | 319 | 13.270 | 1.181 | 44.996 | 1.00 | 0.63 | 1SG | 2601 |
| ATOM | 2601 | CB | GLN | 319 | 12.529 | −0.165 | 44.893 | 1.00 | 0.63 | 1SG | 2602 |
| ATOM | 2602 | CG | GLN | 319 | 12.230 | −0.611 | 43.460 | 1.00 | 0.63 | 1SG | 2603 |
| ATOM | 2603 | CD | GLN | 319 | 13.522 | −1.119 | 42.837 | 1.00 | 0.63 | 1SG | 2604 |
| ATOM | 2604 | OE1 | GLN | 319 | 13.518 | −1.680 | 41.742 | 1.00 | 0.63 | 1SG | 2605 |
| ATOM | 2605 | NE2 | GLN | 319 | 14.661 | −0.924 | 43.553 | 1.00 | 0.63 | 1SG | 2606 |
| ATOM | 2606 | C | GLN | 319 | 12.398 | 2.209 | 44.348 | 1.00 | 0.63 | 1SG | 2607 |
| ATOM | 2607 | O | GLN | 319 | 12.607 | 2.569 | 43.190 | 1.00 | 0.63 | 1SG | 2608 |
| ATOM | 2608 | N | SER | 320 | 11.386 | 2.701 | 45.089 | 1.00 | 0.62 | 1SG | 2609 |
| ATOM | 2609 | CA | SER | 320 | 10.436 | 3.641 | 44.558 | 1.00 | 0.62 | 1SG | 2610 |
| ATOM | 2610 | CB | SER | 320 | 8.992 | 3.129 | 44.667 | 1.00 | 0.62 | 1SG | 2611 |
| ATOM | 2611 | OG | SER | 320 | 8.073 | 4.198 | 44.507 | 1.00 | 0.62 | 1SG | 2612 |
| ATOM | 2612 | C | SER | 320 | 10.495 | 4.885 | 45.378 | 1.00 | 0.62 | 1SG | 2613 |
| ATOM | 2613 | O | SER | 320 | 10.399 | 4.833 | 46.603 | 1.00 | 0.62 | 1SG | 2614 |
| ATOM | 2614 | N | LYS | 321 | 10.631 | 6.056 | 44.724 | 1.00 | 0.65 | 1SG | 2615 |
| ATOM | 2615 | CA | LYS | 321 | 10.707 | 7.238 | 45.527 | 1.00 | 0.65 | 1SG | 2616 |
| ATOM | 2616 | CB | LYS | 321 | 12.155 | 7.607 | 45.857 | 1.00 | 0.65 | 1SG | 2617 |
| ATOM | 2617 | CG | LYS | 321 | 12.864 | 6.594 | 46.757 | 1.00 | 0.65 | 1SG | 2618 |
| ATOM | 2618 | CD | LYS | 321 | 14.379 | 6.794 | 46.816 | 1.00 | 0.65 | 1SG | 2619 |
| ATOM | 2619 | CE | LYS | 321 | 15.107 | 6.318 | 45.558 | 1.00 | 0.65 | 1SG | 2620 |
| ATOM | 2620 | NZ | LYS | 321 | 14.865 | 7.262 | 44.444 | 1.00 | 0.65 | 1SG | 2621 |
| ATOM | 2621 | C | LYS | 321 | 10.098 | 8.379 | 44.797 | 1.00 | 0.65 | 1SG | 2622 |
| ATOM | 2622 | O | LYS | 321 | 9.982 | 8.361 | 43.578 | 1.00 | 0.65 | 1SG | 2623 |
| ATOM | 2623 | N | THR | 322 | 9.675 | 9.423 | 45.533 | 1.00 | 0.68 | 1SG | 2624 |
| ATOM | 2624 | CA | THR | 322 | 9.055 | 10.543 | 44.892 | 1.00 | 0.68 | 1SG | 2625 |
| ATOM | 2625 | CB | THR | 322 | 7.673 | 10.821 | 45.391 | 1.00 | 0.68 | 1SG | 2626 |
| ATOM | 2626 | OG1 | THR | 322 | 7.113 | 11.939 | 44.720 | 1.00 | 0.68 | 1SG | 2627 |
| ATOM | 2627 | CG2 | THR | 322 | 7.722 | 11.039 | 46.907 | 1.00 | 0.68 | 1SG | 2628 |
| ATOM | 2628 | C | THR | 322 | 9.915 | 11.754 | 45.069 | 1.00 | 0.68 | 1SG | 2629 |
| ATOM | 2629 | O | THR | 322 | 10.424 | 12.041 | 46.152 | 1.00 | 0.68 | 1SG | 2630 |
| ATOM | 2630 | N | ARG | 323 | 10.105 | 12.479 | 43.955 | 1.00 | 0.52 | 1SG | 2631 |
| ATOM | 2631 | CA | ARG | 323 | 10.882 | 13.677 | 43.864 | 1.00 | 0.52 | 1SG | 2632 |
| ATOM | 2632 | CB | ARG | 323 | 11.670 | 13.645 | 42.553 | 1.00 | 0.52 | 1SG | 2633 |
| ATOM | 2633 | CG | ARG | 323 | 12.574 | 14.823 | 42.236 | 1.00 | 0.52 | 1SG | 2634 |
| ATOM | 2634 | CD | ARG | 323 | 13.195 | 14.616 | 40.858 | 1.00 | 0.52 | 1SG | 2635 |
| ATOM | 2635 | NE | ARG | 323 | 14.166 | 15.705 | 40.611 | 1.00 | 0.52 | 1SG | 2636 |
| ATOM | 2636 | CZ | ARG | 323 | 15.162 | 15.503 | 39.703 | 1.00 | 0.52 | 1SG | 2637 |
| ATOM | 2637 | NH1 | ARG | 323 | 15.251 | 14.302 | 39.066 | 1.00 | 0.52 | 1SG | 2638 |
| ATOM | 2638 | NH2 | ARG | 323 | 16.085 | 16.476 | 39.456 | 1.00 | 0.52 | 1SG | 2639 |
| ATOM | 2639 | C | ARG | 323 | 9.856 | 14.764 | 43.780 | 1.00 | 0.52 | 1SG | 2640 |
| ATOM | 2640 | O | ARG | 323 | 9.057 | 14.789 | 42.845 | 1.00 | 0.52 | 1SG | 2641 |
| ATOM | 2641 | N | LEU | 324 | 9.859 | 15.695 | 44.757 | 1.00 | 0.36 | 1SG | 2642 |
| ATOM | 2642 | CA | LEU | 324 | 8.822 | 16.686 | 44.851 | 1.00 | 0.36 | 1SG | 2643 |
| ATOM | 2643 | CB | LEU | 324 | 8.466 | 16.992 | 46.313 | 1.00 | 0.36 | 1SG | 2644 |
| ATOM | 2644 | CG | LEU | 324 | 7.370 | 18.049 | 46.505 | 1.00 | 0.36 | 1SG | 2645 |
| ATOM | 2645 | CD2 | LEU | 324 | 7.316 | 18.556 | 47.952 | 1.00 | 0.36 | 1SG | 2646 |
| ATOM | 2646 | CD1 | LEU | 324 | 6.016 | 17.519 | 46.013 | 1.00 | 0.36 | 1SG | 2647 |
| ATOM | 2647 | C | LEU | 324 | 9.288 | 17.979 | 44.247 | 1.00 | 0.36 | 1SG | 2648 |
| ATOM | 2648 | O | LEU | 324 | 10.408 | 18.420 | 44.503 | 1.00 | 0.36 | 1SG | 2649 |
| ATOM | 2649 | N | VAL | 325 | 8.424 | 18.615 | 43.419 | 1.00 | 0.61 | 1SG | 2650 |
| ATOM | 2650 | CA | VAL | 325 | 8.731 | 19.897 | 42.841 | 1.00 | 0.61 | 1SG | 2651 |
| ATOM | 2651 | CB | VAL | 325 | 8.809 | 19.882 | 41.343 | 1.00 | 0.61 | 1SG | 2652 |
| ATOM | 2652 | CG1 | VAL | 325 | 10.011 | 19.024 | 40.915 | 1.00 | 0.61 | 1SG | 2653 |
| ATOM | 2653 | CG2 | VAL | 325 | 7.461 | 19.376 | 40.804 | 1.00 | 0.61 | 1SG | 2654 |
| ATOM | 2654 | C | VAL | 325 | 7.627 | 20.847 | 43.217 | 1.00 | 0.61 | 1SG | 2655 |
| ATOM | 2655 | O | VAL | 325 | 6.444 | 20.534 | 43.076 | 1.00 | 0.61 | 1SG | 2656 |
| ATOM | 2656 | N | LYS | 326 | 7.999 | 22.014 | 43.788 | 1.00 | 0.94 | 1SG | 2657 |
| ATOM | 2657 | CA | LYS | 326 | 7.040 | 23.008 | 44.192 | 1.00 | 0.94 | 1SG | 2658 |
| ATOM | 2658 | CB | LYS | 326 | 7.544 | 23.900 | 45.340 | 1.00 | 0.94 | 1SG | 2659 |
| ATOM | 2659 | CG | LYS | 326 | 8.647 | 24.877 | 44.933 | 1.00 | 0.94 | 1SG | 2660 |
| ATOM | 2660 | CD | LYS | 326 | 9.921 | 24.203 | 44.423 | 1.00 | 0.94 | 1SG | 2661 |
| ATOM | 2661 | CE | LYS | 326 | 11.009 | 25.200 | 44.020 | 1.00 | 0.94 | 1SG | 2662 |
| ATOM | 2662 | NZ | LYS | 326 | 11.532 | 25.887 | 45.224 | 1.00 | 0.94 | 1SG | 2663 |
| ATOM | 2663 | C | LYS | 326 | 6.617 | 23.932 | 43.080 | 1.00 | 0.94 | 1SG | 2664 |
| ATOM | 2664 | O | LYS | 326 | 5.429 | 24.218 | 42.929 | 1.00 | 0.94 | 1SG | 2665 |
| ATOM | 2665 | N | TRP | 327 | 7.571 | 24.424 | 42.257 | 1.00 | 0.75 | 1SG | 2666 |
| ATOM | 2666 | CA | TRP | 327 | 7.213 | 25.466 | 41.329 | 1.00 | 0.75 | 1SG | 2667 |
| ATOM | 2667 | CB | TRP | 327 | 7.769 | 26.836 | 41.783 | 1.00 | 0.75 | 1SG | 2668 |
| ATOM | 2668 | CG | TRP | 327 | 7.321 | 28.050 | 41.000 | 1.00 | 0.75 | 1SG | 2669 |
| ATOM | 2669 | CD2 | TRP | 327 | 6.089 | 28.749 | 41.237 | 1.00 | 0.75 | 1SG | 2670 |
| ATOM | 2670 | CD1 | TRP | 327 | 7.944 | 28.701 | 39.976 | 1.00 | 0.75 | 1SG | 2671 |
| ATOM | 2671 | NE1 | TRP | 327 | 7.178 | 29.764 | 39.560 | 1.00 | 0.75 | 1SG | 2672 |
| ATOM | 2672 | CE2 | TRP | 327 | 6.033 | 29.805 | 40.328 | 1.00 | 0.75 | 1SG | 2673 |
| ATOM | 2673 | CE3 | TRP | 327 | 5.087 | 28.528 | 42.138 | 1.00 | 0.75 | 1SG | 2674 |
| ATOM | 2674 | CZ2 | TRP | 327 | 4.969 | 30.661 | 40.307 | 1.00 | 0.75 | 1SG | 2675 |
| ATOM | 2675 | CZ3 | TRP | 327 | 4.016 | 29.393 | 42.114 | 1.00 | 0.75 | 1SG | 2676 |
| ATOM | 2676 | CH2 | TRP | 327 | 3.958 | 30.439 | 41.216 | 1.00 | 0.75 | 1SG | 2677 |
| ATOM | 2677 | C | TRP | 327 | 7.752 | 25.136 | 39.967 | 1.00 | 0.75 | 1SG | 2678 |
| ATOM | 2678 | O | TRP | 327 | 8.297 | 24.058 | 39.744 | 1.00 | 0.75 | 1SG | 2679 |
| ATOM | 2679 | N | ASN | 328 | 7.574 | 26.072 | 39.010 | 1.00 | 0.47 | 1SG | 2680 |
| ATOM | 2680 | CA | ASN | 328 | 7.971 | 25.915 | 37.640 | 1.00 | 0.47 | 1SG | 2681 |
| ATOM | 2681 | CB | ASN | 328 | 7.657 | 27.160 | 36.792 | 1.00 | 0.47 | 1SG | 2682 |
| ATOM | 2682 | CG | ASN | 328 | 6.152 | 27.381 | 36.784 | 1.00 | 0.47 | 1SG | 2683 |
| ATOM | 2683 | OD1 | ASN | 328 | 5.543 | 27.582 | 37.834 | 1.00 | 0.47 | 1SG | 2684 |
| ATOM | 2684 | ND2 | ASN | 328 | 5.538 | 27.357 | 35.570 | 1.00 | 0.47 | 1SG | 2685 |
| ATOM | 2685 | C | ASN | 328 | 9.453 | 25.726 | 37.566 | 1.00 | 0.47 | 1SG | 2686 |
| ATOM | 2686 | O | ASN | 328 | 9.939 | 24.837 | 36.868 | 1.00 | 0.47 | 1SG | 2687 |
| ATOM | 2687 | N | TYR | 329 | 10.215 | 26.565 | 38.290 | 1.00 | 0.54 | 1SG | 2688 |
| ATOM | 2688 | CA | TYR | 329 | 11.647 | 26.501 | 38.222 | 1.00 | 0.54 | 1SG | 2689 |
| ATOM | 2689 | CB | TYR | 329 | 12.333 | 27.584 | 39.075 | 1.00 | 0.54 | 1SG | 2690 |
| ATOM | 2690 | CG | TYR | 329 | 11.994 | 28.909 | 38.483 | 1.00 | 0.54 | 1SG | 2691 |
| ATOM | 2691 | CD1 | TYR | 329 | 10.800 | 29.525 | 38.784 | 1.00 | 0.54 | 1SG | 2692 |
| ATOM | 2692 | CD2 | TYR | 329 | 12.870 | 29.537 | 37.628 | 1.00 | 0.54 | 1SG | 2693 |
| ATOM | 2693 | CE1 | TYR | 329 | 10.486 | 30.748 | 38.240 | 1.00 | 0.54 | 1SG | 2694 |
| ATOM | 2694 | CE2 | TYR | 329 | 12.561 | 30.760 | 37.081 | 1.00 | 0.54 | 1SG | 2695 |
| ATOM | 2695 | CZ | TYR | 329 | 11.367 | 31.368 | 37.387 | 1.00 | 0.54 | 1SG | 2696 |
| ATOM | 2696 | OH | TYR | 329 | 11.046 | 32.623 | 36.827 | 1.00 | 0.54 | 1SG | 2697 |
| ATOM | 2697 | C | TYR | 329 | 12.109 | 25.172 | 38.728 | 1.00 | 0.54 | 1SG | 2698 |
| ATOM | 2698 | O | TYR | 329 | 12.980 | 24.540 | 38.132 | 1.00 | 0.54 | 1SG | 2699 |
| ATOM | 2699 | N | TYR | 330 | 11.521 | 24.714 | 39.845 | 1.00 | 0.71 | 1SG | 2700 |
| ATOM | 2700 | CA | TYR | 330 | 11.921 | 23.485 | 40.467 | 1.00 | 0.71 | 1SG | 2701 |
| ATOM | 2701 | CB | TYR | 330 | 11.118 | 23.248 | 41.761 | 1.00 | 0.71 | 1SG | 2702 |
| ATOM | 2702 | CG | TYR | 330 | 11.793 | 22.260 | 42.651 | 1.00 | 0.71 | 1SG | 2703 |
| ATOM | 2703 | CD1 | TYR | 330 | 11.668 | 20.909 | 42.446 | 1.00 | 0.71 | 1SG | 2704 |
| ATOM | 2704 | CD2 | TYR | 330 | 12.543 | 22.698 | 43.719 | 1.00 | 0.71 | 1SG | 2705 |
| ATOM | 2705 | CE1 | TYR | 330 | 12.287 | 20.008 | 43.282 | 1.00 | 0.71 | 1SG | 2706 |
| ATOM | 2706 | CE2 | TYR | 330 | 13.165 | 21.806 | 44.560 | 1.00 | 0.71 | 1SG | 2707 |
| ATOM | 2707 | CZ | TYR | 330 | 13.036 | 20.456 | 44.343 | 1.00 | 0.71 | 1SG | 2708 |
| ATOM | 2708 | OH | TYR | 330 | 13.672 | 19.535 | 45.202 | 1.00 | 0.71 | 1SG | 2709 |
| ATOM | 2709 | C | TYR | 330 | 11.635 | 22.372 | 39.500 | 1.00 | 0.71 | 1SG | 2710 |
| ATOM | 2710 | O | TYR | 330 | 12.468 | 21.496 | 39.280 | 1.00 | 0.71 | 1SG | 2711 |
| ATOM | 2711 | N | GLU | 331 | 10.456 | 22.393 | 38.854 | 1.00 | 0.65 | 1SG | 2712 |
| ATOM | 2712 | CA | GLU | 331 | 10.125 | 21.330 | 37.948 | 1.00 | 0.65 | 1SG | 2713 |
| ATOM | 2713 | CB | GLU | 331 | 8.787 | 21.559 | 37.224 | 1.00 | 0.65 | 1SG | 2714 |
| ATOM | 2714 | CG | GLU | 331 | 7.543 | 21.310 | 38.074 | 1.00 | 0.65 | 1SG | 2715 |
| ATOM | 2715 | CD | GLU | 331 | 7.171 | 19.844 | 37.907 | 1.00 | 0.65 | 1SG | 2716 |
| ATOM | 2716 | OE1 | GLU | 331 | 7.974 | 19.099 | 37.284 | 1.00 | 0.65 | 1SG | 2717 |
| ATOM | 2717 | OE2 | GLU | 331 | 6.081 | 19.449 | 38.399 | 1.00 | 0.65 | 1SG | 2718 |
| ATOM | 2718 | C | GLU | 331 | 11.167 | 21.289 | 36.877 | 1.00 | 0.65 | 1SG | 2719 |
| ATOM | 2719 | O | GLU | 331 | 11.716 | 20.233 | 36.562 | 1.00 | 0.65 | 1SG | 2720 |
| ATOM | 2720 | N | GLY | 332 | 11.482 | 22.469 | 36.320 | 1.00 | 0.53 | 1SG | 2721 |
| ATOM | 2721 | CA | GLY | 332 | 12.392 | 22.596 | 35.223 | 1.00 | 0.53 | 1SG | 2722 |
| ATOM | 2722 | C | GLY | 332 | 13.799 | 22.274 | 35.610 | 1.00 | 0.53 | 1SG | 2723 |
| ATOM | 2723 | O | GLY | 332 | 14.617 | 22.039 | 34.729 | 1.00 | 0.53 | 1SG | 2724 |
| ATOM | 2724 | N | PHE | 333 | 14.170 | 22.406 | 36.897 | 1.00 | 0.49 | 1SG | 2725 |
| ATOM | 2725 | CA | PHE | 333 | 15.489 | 22.001 | 37.313 | 1.00 | 0.49 | 1SG | 2726 |
| ATOM | 2726 | CB | PHE | 333 | 15.910 | 22.632 | 38.650 | 1.00 | 0.49 | 1SG | 2727 |
| ATOM | 2727 | CG | PHE | 333 | 17.257 | 22.088 | 38.979 | 1.00 | 0.49 | 1SG | 2728 |
| ATOM | 2728 | CD1 | PHE | 333 | 18.394 | 22.638 | 38.434 | 1.00 | 0.49 | 1SG | 2729 |
| ATOM | 2729 | CD2 | PHE | 333 | 17.379 | 21.019 | 39.837 | 1.00 | 0.49 | 1SG | 2730 |
| ATOM | 2730 | CE1 | PHE | 333 | 19.634 | 22.129 | 38.742 | 1.00 | 0.49 | 1SG | 2731 |
| ATOM | 2731 | CE2 | PHE | 333 | 18.616 | 20.506 | 40.149 | 1.00 | 0.49 | 1SG | 2732 |
| ATOM | 2732 | CZ | PHE | 333 | 19.747 | 21.062 | 39.601 | 1.00 | 0.49 | 1SG | 2733 |
| ATOM | 2733 | C | PHE | 333 | 15.548 | 20.516 | 37.498 | 1.00 | 0.49 | 1SG | 2734 |
| ATOM | 2734 | O | PHE | 333 | 16.521 | 19.852 | 37.136 | 1.00 | 0.49 | 1SG | 2735 |
| ATOM | 2735 | N | PHE | 334 | 14.492 | 19.970 | 38.123 | 1.00 | 0.49 | 1SG | 2736 |
| ATOM | 2736 | CA | PHE | 334 | 14.452 | 18.584 | 38.468 | 1.00 | 0.49 | 1SG | 2737 |
| ATOM | 2737 | CB | PHE | 334 | 13.520 | 18.329 | 39.664 | 1.00 | 0.49 | 1SG | 2738 |
| ATOM | 2738 | CG | PHE | 334 | 14.246 | 18.963 | 40.813 | 1.00 | 0.49 | 1SG | 2739 |
| ATOM | 2739 | CD1 | PHE | 334 | 14.187 | 20.318 | 41.029 | 1.00 | 0.49 | 1SG | 2740 |
| ATOM | 2740 | CD2 | PHE | 334 | 15.010 | 18.218 | 41.678 | 1.00 | 0.49 | 1SG | 2741 |
| ATOM | 2741 | CE1 | PHE | 334 | 14.851 | 20.922 | 42.068 | 1.00 | 0.49 | 1SG | 2742 |
| ATOM | 2742 | CE2 | PHE | 334 | 15.681 | 18.806 | 42.725 | 1.00 | 0.49 | 1SG | 2743 |
| ATOM | 2743 | CZ | PHE | 334 | 15.603 | 20.162 | 42.927 | 1.00 | 0.49 | 1SG | 2744 |
| ATOM | 2744 | C | PHE | 334 | 14.152 | 17.704 | 37.288 | 1.00 | 0.49 | 1SG | 2745 |
| ATOM | 2745 | O | PHE | 334 | 14.634 | 16.572 | 37.234 | 1.00 | 0.49 | 1SG | 2746 |
| ATOM | 2746 | N | TYR | 335 | 13.345 | 18.170 | 36.311 | 1.00 | 0.74 | 1SG | 2747 |
| ATOM | 2747 | CA | TYR | 335 | 13.152 | 17.318 | 35.165 | 1.00 | 0.74 | 1SG | 2748 |
| ATOM | 2748 | CB | TYR | 335 | 12.139 | 17.812 | 34.070 | 1.00 | 0.74 | 1SG | 2749 |
| ATOM | 2749 | CG | TYR | 335 | 12.720 | 18.911 | 33.246 | 1.00 | 0.74 | 1SG | 2750 |
| ATOM | 2750 | CD1 | TYR | 335 | 13.619 | 18.625 | 32.242 | 1.00 | 0.74 | 1SG | 2751 |
| ATOM | 2751 | CD2 | TYR | 335 | 12.385 | 20.226 | 33.463 | 1.00 | 0.74 | 1SG | 2752 |
| ATOM | 2752 | CE1 | TYR | 335 | 14.200 | 19.611 | 31.492 | 1.00 | 0.74 | 1SG | 2753 |
| ATOM | 2753 | CE2 | TYR | 335 | 12.959 | 21.219 | 32.703 | 1.00 | 0.74 | 1SG | 2754 |
| ATOM | 2754 | CZ | TYR | 335 | 13.873 | 20.922 | 31.730 | 1.00 | 0.74 | 1SG | 2755 |
| ATOM | 2755 | OH | TYR | 335 | 14.458 | 21.953 | 30.964 | 1.00 | 0.74 | 1SG | 2756 |
| ATOM | 2756 | C | TYR | 335 | 14.500 | 17.045 | 34.542 | 1.00 | 0.74 | 1SG | 2757 |
| ATOM | 2757 | O | TYR | 335 | 14.796 | 15.874 | 34.313 | 1.00 | 0.74 | 1SG | 2758 |
| ATOM | 2758 | N | PRO | 336 | 15.382 | 18.003 | 34.257 | 1.00 | 0.98 | 1SG | 2759 |
| ATOM | 2759 | CA | PRO | 336 | 16.676 | 17.722 | 33.742 | 1.00 | 0.98 | 1SG | 2760 |
| ATOM | 2760 | CD | PRO | 336 | 15.314 | 19.388 | 34.642 | 1.00 | 0.98 | 1SG | 2761 |
| ATOM | 2761 | CB | PRO | 336 | 17.274 | 19.049 | 33.300 | 1.00 | 0.98 | 1SG | 2762 |
| ATOM | 2762 | CG | PRO | 336 | 16.678 | 20.016 | 34.323 | 1.00 | 0.98 | 1SG | 2763 |
| ATOM | 2763 | C | PRO | 336 | 17.312 | 17.273 | 35.003 | 1.00 | 0.98 | 1SG | 2764 |
| ATOM | 2764 | O | PRO | 336 | 16.705 | 17.388 | 36.060 | 1.00 | 0.98 | 1SG | 2765 |
| ATOM | 2765 | N | SER | 337 | 18.543 | 16.798 | 34.947 | 1.00 | 0.99 | 1SG | 2766 |
| ATOM | 2766 | CA | SER | 337 | 19.161 | 16.193 | 36.083 | 1.00 | 0.99 | 1SG | 2767 |
| ATOM | 2767 | CB | SER | 337 | 18.967 | 16.878 | 37.457 | 1.00 | 0.99 | 1SG | 2768 |
| ATOM | 2768 | OG | SER | 337 | 19.563 | 18.166 | 37.449 | 1.00 | 0.99 | 1SG | 2769 |
| ATOM | 2769 | C | SER | 337 | 18.624 | 14.798 | 36.117 | 1.00 | 0.99 | 1SG | 2770 |
| ATOM | 2770 | O | SER | 337 | 19.385 | 13.839 | 36.205 | 1.00 | 0.99 | 1SG | 2771 |
| ATOM | 2771 | N | CYS | 338 | 17.287 | 14.650 | 36.033 | 1.00 | 0.78 | 1SG | 2772 |
| ATOM | 2772 | CA | CYS | 338 | 16.672 | 13.355 | 35.977 | 1.00 | 0.78 | 1SG | 2773 |
| ATOM | 2773 | CB | CYS | 338 | 15.187 | 13.376 | 36.362 | 1.00 | 0.78 | 1SG | 2774 |
| ATOM | 2774 | SG | CYS | 338 | 14.449 | 11.721 | 36.278 | 1.00 | 0.78 | 1SG | 2775 |
| ATOM | 2775 | C | CYS | 338 | 16.734 | 12.843 | 34.568 | 1.00 | 0.78 | 1SG | 2776 |
| ATOM | 2776 | O | CYS | 338 | 16.742 | 11.633 | 34.346 | 1.00 | 0.78 | 1SG | 2777 |
| ATOM | 2777 | N | THR | 339 | 16.751 | 13.768 | 33.585 | 1.00 | 0.49 | 1SG | 2778 |
| ATOM | 2778 | CA | THR | 339 | 16.798 | 13.441 | 32.181 | 1.00 | 0.49 | 1SG | 2779 |
| ATOM | 2779 | CB | THR | 339 | 17.706 | 12.278 | 31.868 | 1.00 | 0.49 | 1SG | 2780 |
| ATOM | 2780 | OG1 | THR | 339 | 19.002 | 12.536 | 32.387 | 1.00 | 0.49 | 1SG | 2781 |
| ATOM | 2781 | CG2 | THR | 339 | 17.814 | 12.109 | 30.342 | 1.00 | 0.49 | 1SG | 2782 |
| ATOM | 2782 | C | THR | 339 | 15.399 | 13.156 | 31.706 | 1.00 | 0.49 | 1SG | 2783 |
| ATOM | 2783 | O | THR | 339 | 15.149 | 12.236 | 30.928 | 1.00 | 0.49 | 1SG | 2784 |
| ATOM | 2784 | N | GLY | 340 | 14.433 | 13.972 | 32.170 | 1.00 | 0.49 | 1SG | 2785 |
| ATOM | 2785 | CA | GLY | 340 | 13.071 | 13.823 | 31.743 | 1.00 | 0.49 | 1SG | 2786 |
| ATOM | 2786 | C | GLY | 340 | 12.777 | 14.970 | 30.833 | 1.00 | 0.49 | 1SG | 2787 |
| ATOM | 2787 | O | GLY | 340 | 13.441 | 16.004 | 30.890 | 1.00 | 0.49 | 1SG | 2788 |
| ATOM | 2788 | N | SER | 341 | 11.753 | 14.815 | 29.972 | 1.00 | 0.74 | 1SG | 2789 |
| ATOM | 2789 | CA | SER | 341 | 11.416 | 15.845 | 29.032 | 1.00 | 0.74 | 1SG | 2790 |
| ATOM | 2790 | CB | SER | 341 | 10.206 | 15.503 | 28.145 | 1.00 | 0.74 | 1SG | 2791 |
| ATOM | 2791 | OG | SER | 341 | 9.033 | 15.398 | 28.939 | 1.00 | 0.74 | 1SG | 2792 |
| ATOM | 2792 | C | SER | 341 | 11.092 | 17.093 | 29.786 | 1.00 | 0.74 | 1SG | 2793 |
| ATOM | 2793 | O | SER | 341 | 10.287 | 17.092 | 30.717 | 1.00 | 0.74 | 1SG | 2794 |
| ATOM | 2794 | N | HIS | 342 | 11.760 | 18.181 | 29.359 | 1.00 | 0.91 | 1SG | 2795 |
| ATOM | 2795 | CA | HIS | 342 | 11.754 | 19.531 | 29.851 | 1.00 | 0.91 | 1SG | 2796 |
| ATOM | 2796 | ND1 | HIS | 342 | 11.949 | 21.084 | 26.763 | 1.00 | 0.91 | 1SG | 2797 |
| ATOM | 2797 | CG | HIS | 342 | 12.696 | 20.255 | 27.570 | 1.00 | 0.91 | 1SG | 2798 |
| ATOM | 2798 | CB | HIS | 342 | 12.780 | 20.379 | 29.066 | 1.00 | 0.91 | 1SG | 2799 |
| ATOM | 2799 | NE2 | HIS | 342 | 12.937 | 19.612 | 25.420 | 1.00 | 0.91 | 1SG | 2800 |
| ATOM | 2800 | CD2 | HIS | 342 | 13.293 | 19.362 | 26.734 | 1.00 | 0.91 | 1SG | 2801 |
| ATOM | 2801 | CE1 | HIS | 342 | 12.129 | 20.655 | 25.488 | 1.00 | 0.91 | 1SG | 2802 |
| ATOM | 2802 | C | HIS | 342 | 10.465 | 20.243 | 29.602 | 1.00 | 0.91 | 1SG | 2803 |
| ATOM | 2803 | O | HIS | 342 | 9.930 | 20.907 | 30.489 | 1.00 | 0.91 | 1SG | 2804 |
| ATOM | 2804 | N | LEU | 343 | 9.954 | 20.107 | 28.369 | 1.00 | 0.87 | 1SG | 2805 |
| ATOM | 2805 | CA | LEU | 343 | 8.903 | 20.920 | 27.840 | 1.00 | 0.87 | 1SG | 2806 |
| ATOM | 2806 | CB | LEU | 343 | 8.596 | 20.592 | 26.370 | 1.00 | 0.87 | 1SG | 2807 |
| ATOM | 2807 | CG | LEU | 343 | 7.494 | 21.481 | 25.786 | 1.00 | 0.87 | 1SG | 2808 |
| ATOM | 2808 | CD2 | LEU | 343 | 7.010 | 20.972 | 24.421 | 1.00 | 0.87 | 1SG | 2809 |
| ATOM | 2809 | CD1 | LEU | 343 | 7.968 | 22.939 | 25.754 | 1.00 | 0.87 | 1SG | 2810 |
| ATOM | 2810 | C | LEU | 343 | 7.632 | 20.770 | 28.599 | 1.00 | 0.87 | 1SG | 2811 |
| ATOM | 2811 | O | LEU | 343 | 7.399 | 19.775 | 29.282 | 1.00 | 0.87 | 1SG | 2812 |
| ATOM | 2812 | N | ARG | 344 | 6.775 | 21.808 | 28.502 | 1.00 | 0.69 | 1SG | 2813 |
| ATOM | 2813 | CA | ARG | 344 | 5.501 | 21.758 | 29.147 | 1.00 | 0.69 | 1SG | 2814 |
| ATOM | 2814 | CB | ARG | 344 | 5.526 | 22.400 | 30.546 | 1.00 | 0.69 | 1SG | 2815 |
| ATOM | 2815 | CG | ARG | 344 | 4.230 | 22.244 | 31.339 | 1.00 | 0.69 | 1SG | 2816 |
| ATOM | 2816 | CD | ARG | 344 | 4.477 | 22.156 | 32.848 | 1.00 | 0.69 | 1SG | 2817 |
| ATOM | 2817 | NE | ARG | 344 | 5.820 | 22.745 | 33.115 | 1.00 | 0.69 | 1SG | 2818 |
| ATOM | 2818 | CZ | ARG | 344 | 6.523 | 22.369 | 34.224 | 1.00 | 0.69 | 1SG | 2819 |
| ATOM | 2819 | NH1 | ARG | 344 | 6.004 | 21.445 | 35.083 | 1.00 | 0.69 | 1SG | 2820 |
| ATOM | 2820 | NH2 | ARG | 344 | 7.741 | 22.929 | 34.481 | 1.00 | 0.69 | 1SG | 2821 |
| ATOM | 2821 | C | ARG | 344 | 4.521 | 22.458 | 28.266 | 1.00 | 0.69 | 1SG | 2822 |
| ATOM | 2822 | O | ARG | 344 | 4.818 | 23.502 | 27.689 | 1.00 | 0.69 | 1SG | 2823 |
| ATOM | 2823 | N | SER | 345 | 3.323 | 21.868 | 28.107 | 1.00 | 0.58 | 1SG | 2824 |
| ATOM | 2824 | CA | SER | 345 | 2.322 | 22.466 | 27.277 | 1.00 | 0.58 | 1SG | 2825 |
| ATOM | 2825 | CB | SER | 345 | 1.793 | 21.535 | 26.176 | 1.00 | 0.58 | 1SG | 2826 |
| ATOM | 2826 | OG | SER | 345 | 0.809 | 22.204 | 25.400 | 1.00 | 0.58 | 1SG | 2827 |
| ATOM | 2827 | C | SER | 345 | 1.176 | 22.817 | 28.155 | 1.00 | 0.58 | 1SG | 2828 |
| ATOM | 2828 | O | SER | 345 | 0.730 | 22.004 | 28.957 | 1.00 | 0.58 | 1SG | 2829 |
| ATOM | 2829 | N | VAL | 346 | 0.669 | 24.050 | 28.007 | 1.00 | 0.61 | 1SG | 2830 |
| ATOM | 2830 | CA | VAL | 346 | −0.410 | 24.563 | 28.795 | 1.00 | 0.61 | 1SG | 2831 |
| ATOM | 2831 | CB | VAL | 346 | −1.783 | 24.091 | 28.383 | 1.00 | 0.61 | 1SG | 2832 |
| ATOM | 2832 | CG1 | VAL | 346 | −2.035 | 24.553 | 26.938 | 1.00 | 0.61 | 1SG | 2833 |
| ATOM | 2833 | CG2 | VAL | 346 | −1.919 | 22.576 | 28.598 | 1.00 | 0.61 | 1SG | 2834 |
| ATOM | 2834 | C | VAL | 346 | −0.190 | 24.242 | 30.238 | 1.00 | 0.61 | 1SG | 2835 |
| ATOM | 2835 | O | VAL | 346 | −1.135 | 23.937 | 30.964 | 1.00 | 0.61 | 1SG | 2836 |
| ATOM | 2836 | N | CYS | 347 | 1.080 | 24.325 | 30.686 | 1.00 | 0.63 | 1SG | 2837 |
| ATOM | 2837 | CA | CYS | 347 | 1.418 | 24.150 | 32.071 | 1.00 | 0.63 | 1SG | 2838 |
| ATOM | 2838 | CB | CYS | 347 | 0.426 | 24.893 | 32.998 | 1.00 | 0.63 | 1SG | 2839 |
| ATOM | 2839 | SG | CYS | 347 | 0.779 | 24.769 | 34.779 | 1.00 | 0.63 | 1SG | 2840 |
| ATOM | 2840 | C | CYS | 347 | 1.473 | 22.696 | 32.451 | 1.00 | 0.63 | 1SG | 2841 |
| ATOM | 2841 | O | CYS | 347 | 1.773 | 22.369 | 33.596 | 1.00 | 0.63 | 1SG | 2842 |
| ATOM | 2842 | N | ILE | 348 | 1.232 | 21.758 | 31.516 | 1.00 | 0.61 | 1SG | 2843 |
| ATOM | 2843 | CA | ILE | 348 | 1.328 | 20.382 | 31.925 | 1.00 | 0.61 | 1SG | 2844 |
| ATOM | 2844 | CB | ILE | 348 | 0.217 | 19.503 | 31.461 | 1.00 | 0.61 | 1SG | 2845 |
| ATOM | 2845 | CG2 | ILE | 348 | 0.177 | 19.509 | 29.929 | 1.00 | 0.61 | 1SG | 2846 |
| ATOM | 2846 | CG1 | ILE | 348 | 0.421 | 18.109 | 32.064 | 1.00 | 0.61 | 1SG | 2847 |
| ATOM | 2847 | CD1 | ILE | 348 | −0.756 | 17.182 | 31.815 | 1.00 | 0.61 | 1SG | 2848 |
| ATOM | 2848 | C | ILE | 348 | 2.578 | 19.799 | 31.355 | 1.00 | 0.61 | 1SG | 2849 |
| ATOM | 2849 | O | ILE | 348 | 2.910 | 20.041 | 30.198 | 1.00 | 0.61 | 1SG | 2850 |
| ATOM | 2850 | N | TYR | 349 | 3.325 | 19.008 | 32.154 | 1.00 | 0.56 | 1SG | 2851 |
| ATOM | 2851 | CA | TYR | 349 | 4.531 | 18.482 | 31.585 | 1.00 | 0.56 | 1SG | 2852 |
| ATOM | 2852 | CB | TYR | 349 | 5.785 | 18.539 | 32.479 | 1.00 | 0.56 | 1SG | 2853 |
| ATOM | 2853 | CG | TYR | 349 | 5.590 | 17.812 | 33.759 | 1.00 | 0.56 | 1SG | 2854 |
| ATOM | 2854 | CD1 | TYR | 349 | 5.668 | 16.441 | 33.814 | 1.00 | 0.56 | 1SG | 2855 |
| ATOM | 2855 | CD2 | TYR | 349 | 5.354 | 18.519 | 34.915 | 1.00 | 0.56 | 1SG | 2856 |
| ATOM | 2856 | CE1 | TYR | 349 | 5.503 | 15.787 | 35.010 | 1.00 | 0.56 | 1SG | 2857 |
| ATOM | 2857 | CE2 | TYR | 349 | 5.189 | 17.870 | 36.114 | 1.00 | 0.56 | 1SG | 2858 |
| ATOM | 2858 | CZ | TYR | 349 | 5.264 | 16.500 | 36.159 | 1.00 | 0.56 | 1SG | 2859 |
| ATOM | 2859 | OH | TYR | 349 | 5.100 | 15.826 | 37.385 | 1.00 | 0.56 | 1SG | 2860 |
| ATOM | 2860 | C | TYR | 349 | 4.321 | 17.104 | 31.056 | 1.00 | 0.56 | 1SG | 2861 |
| ATOM | 2861 | O | TYR | 349 | 3.275 | 16.486 | 31.251 | 1.00 | 0.56 | 1SG | 2862 |
| ATOM | 2862 | N | GLY | 350 | 5.353 | 16.607 | 30.346 | 1.00 | 0.48 | 1SG | 2863 |
| ATOM | 2863 | CA | GLY | 350 | 5.313 | 15.359 | 29.647 | 1.00 | 0.48 | 1SG | 2864 |
| ATOM | 2864 | C | GLY | 350 | 4.955 | 14.248 | 30.577 | 1.00 | 0.48 | 1SG | 2865 |
| ATOM | 2865 | O | GLY | 350 | 5.528 | 14.095 | 31.655 | 1.00 | 0.48 | 1SG | 2866 |
| ATOM | 2866 | N | ALA | 351 | 3.984 | 13.428 | 30.135 | 1.00 | 0.41 | 1SG | 2867 |
| ATOM | 2867 | CA | ALA | 351 | 3.546 | 12.259 | 30.838 | 1.00 | 0.41 | 1SG | 2868 |
| ATOM | 2868 | CB | ALA | 351 | 4.669 | 11.226 | 31.041 | 1.00 | 0.41 | 1SG | 2869 |
| ATOM | 2869 | C | ALA | 351 | 3.005 | 12.624 | 32.182 | 1.00 | 0.41 | 1SG | 2870 |
| ATOM | 2870 | O | ALA | 351 | 2.831 | 11.752 | 33.031 | 1.00 | 0.41 | 1SG | 2871 |
| ATOM | 2871 | N | ALA | 352 | 2.686 | 13.910 | 32.417 | 1.00 | 0.46 | 1SG | 2872 |
| ATOM | 2872 | CA | ALA | 352 | 2.167 | 14.261 | 33.710 | 1.00 | 0.46 | 1SG | 2873 |
| ATOM | 2873 | CB | ALA | 352 | 1.962 | 15.773 | 33.905 | 1.00 | 0.46 | 1SG | 2874 |
| ATOM | 2874 | C | ALA | 352 | 0.833 | 13.602 | 33.843 | 1.00 | 0.46 | 1SG | 2875 |
| ATOM | 2875 | O | ALA | 352 | 0.070 | 13.552 | 32.879 | 1.00 | 0.46 | 1SG | 2876 |
| ATOM | 2876 | N | GLU | 353 | 0.505 | 13.059 | 35.038 | 1.00 | 0.63 | 1SG | 2877 |
| ATOM | 2877 | CA | GLU | 353 | −0.765 | 12.395 | 35.095 | 1.00 | 0.63 | 1SG | 2878 |
| ATOM | 2878 | CB | GLU | 353 | −0.720 | 10.934 | 34.646 | 1.00 | 0.63 | 1SG | 2879 |
| ATOM | 2879 | CG | GLU | 353 | 0.193 | 10.076 | 35.522 | 1.00 | 0.63 | 1SG | 2880 |
| ATOM | 2880 | CD | GLU | 353 | −0.164 | 8.609 | 35.319 | 1.00 | 0.63 | 1SG | 2881 |
| ATOM | 2881 | OE1 | GLU | 353 | −1.291 | 8.216 | 35.725 | 1.00 | 0.63 | 1SG | 2882 |
| ATOM | 2882 | OE2 | GLU | 353 | 0.674 | 7.867 | 34.741 | 1.00 | 0.63 | 1SG | 2883 |
| ATOM | 2883 | C | GLU | 353 | −1.313 | 12.333 | 36.482 | 1.00 | 0.63 | 1SG | 2884 |
| ATOM | 2884 | O | GLU | 353 | −0.598 | 12.490 | 37.471 | 1.00 | 0.63 | 1SG | 2885 |
| ATOM | 2885 | N | LEU | 354 | −2.636 | 12.059 | 36.535 | 1.00 | 0.83 | 1SG | 2886 |
| ATOM | 2886 | CA | LEU | 354 | −3.460 | 11.929 | 37.703 | 1.00 | 0.83 | 1SG | 2887 |
| ATOM | 2887 | CB | LEU | 354 | −2.724 | 11.657 | 39.028 | 1.00 | 0.83 | 1SG | 2888 |
| ATOM | 2888 | CG | LEU | 354 | −2.232 | 10.202 | 39.156 | 1.00 | 0.83 | 1SG | 2889 |
| ATOM | 2889 | CD2 | LEU | 354 | −1.883 | 9.850 | 40.609 | 1.00 | 0.83 | 1SG | 2890 |
| ATOM | 2890 | CD1 | LEU | 354 | −1.107 | 9.884 | 38.161 | 1.00 | 0.83 | 1SG | 2891 |
| ATOM | 2891 | C | LEU | 354 | −4.254 | 13.177 | 37.833 | 1.00 | 0.83 | 1SG | 2892 |
| ATOM | 2892 | O | LEU | 354 | −4.439 | 13.914 | 36.866 | 1.00 | 0.83 | 1SG | 2893 |
| ATOM | 2893 | N | ARG | 355 | −4.776 | 13.443 | 39.038 | 1.00 | 1.04 | 1SG | 2894 |
| ATOM | 2894 | CA | ARG | 355 | −5.491 | 14.669 | 39.170 | 1.00 | 1.04 | 1SG | 2895 |
| ATOM | 2895 | CB | ARG | 355 | −6.139 | 14.882 | 40.553 | 1.00 | 1.04 | 1SG | 2896 |
| ATOM | 2896 | CG | ARG | 355 | −5.164 | 14.965 | 41.730 | 1.00 | 1.04 | 1SG | 2897 |
| ATOM | 2897 | CD | ARG | 355 | −5.872 | 15.227 | 43.064 | 1.00 | 1.04 | 1SG | 2898 |
| ATOM | 2898 | NE | ARG | 355 | −4.834 | 15.342 | 44.127 | 1.00 | 1.04 | 1SG | 2899 |
| ATOM | 2899 | CZ | ARG | 355 | −5.210 | 15.604 | 45.413 | 1.00 | 1.04 | 1SG | 2900 |
| ATOM | 2900 | NH1 | ARG | 355 | −6.533 | 15.729 | 45.724 | 1.00 | 1.04 | 1SG | 2901 |
| ATOM | 2901 | NH2 | ARG | 355 | −4.265 | 15.741 | 46.388 | 1.00 | 1.04 | 1SG | 2902 |
| ATOM | 2902 | C | ARG | 355 | −4.463 | 15.723 | 38.968 | 1.00 | 1.04 | 1SG | 2903 |
| ATOM | 2903 | O | ARG | 355 | −4.748 | 16.805 | 38.459 | 1.00 | 1.04 | 1SG | 2904 |
| ATOM | 2904 | N | TRP | 356 | −3.214 | 15.395 | 39.353 | 1.00 | 1.11 | 1SG | 2905 |
| ATOM | 2905 | CA | TRP | 356 | −2.136 | 16.327 | 39.278 | 1.00 | 1.11 | 1SG | 2906 |
| ATOM | 2906 | CB | TRP | 356 | −1.446 | 16.612 | 40.623 | 1.00 | 1.11 | 1SG | 2907 |
| ATOM | 2907 | CG | TRP | 356 | −1.367 | 15.454 | 41.588 | 1.00 | 1.11 | 1SG | 2908 |
| ATOM | 2908 | CD2 | TRP | 356 | −0.729 | 14.197 | 41.325 | 1.00 | 1.11 | 1SG | 2909 |
| ATOM | 2909 | CD1 | TRP | 356 | −1.915 | 15.355 | 42.833 | 1.00 | 1.11 | 1SG | 2910 |
| ATOM | 2910 | NE1 | TRP | 356 | −1.624 | 14.130 | 43.380 | 1.00 | 1.11 | 1SG | 2911 |
| ATOM | 2911 | CE2 | TRP | 356 | −0.901 | 13.403 | 42.460 | 1.00 | 1.11 | 1SG | 2912 |
| ATOM | 2912 | CE3 | TRP | 356 | −0.071 | 13.736 | 40.225 | 1.00 | 1.11 | 1SG | 2913 |
| ATOM | 2913 | CZ2 | TRP | 356 | −0.406 | 12.133 | 42.514 | 1.00 | 1.11 | 1SG | 2914 |
| ATOM | 2914 | CZ3 | TRP | 356 | 0.444 | 12.462 | 40.291 | 1.00 | 1.11 | 1SG | 2915 |
| ATOM | 2915 | CH2 | TRP | 356 | 0.283 | 11.678 | 41.413 | 1.00 | 1.11 | 1SG | 2916 |
| ATOM | 2916 | C | TRP | 356 | −1.097 | 15.922 | 38.301 | 1.00 | 1.11 | 1SG | 2917 |
| ATOM | 2917 | O | TRP | 356 | −1.224 | 14.918 | 37.612 | 1.00 | 1.11 | 1SG | 2918 |
| ATOM | 2918 | N | LEU | 357 | −0.032 | 16.743 | 38.208 | 1.00 | 0.94 | 1SG | 2919 |
| ATOM | 2919 | CA | LEU | 357 | 1.022 | 16.530 | 37.262 | 1.00 | 0.94 | 1SG | 2920 |
| ATOM | 2920 | CB | LEU | 357 | 1.671 | 17.834 | 36.770 | 1.00 | 0.94 | 1SG | 2921 |
| ATOM | 2921 | CG | LEU | 357 | 0.700 | 18.770 | 36.028 | 1.00 | 0.94 | 1SG | 2922 |
| ATOM | 2922 | CD2 | LEU | 357 | 1.450 | 19.924 | 35.342 | 1.00 | 0.94 | 1SG | 2923 |
| ATOM | 2923 | CD1 | LEU | 357 | −0.420 | 19.258 | 36.961 | 1.00 | 0.94 | 1SG | 2924 |
| ATOM | 2924 | C | LEU | 357 | 2.111 | 15.724 | 37.886 | 1.00 | 0.94 | 1SG | 2925 |
| ATOM | 2925 | O | LEU | 357 | 2.909 | 16.249 | 38.661 | 1.00 | 0.94 | 1SG | 2926 |
| ATOM | 2926 | N | ILE | 358 | 2.157 | 14.419 | 37.559 | 1.00 | 0.94 | 1SG | 2927 |
| ATOM | 2927 | CA | ILE | 358 | 3.246 | 13.590 | 37.977 | 1.00 | 0.94 | 1SG | 2928 |
| ATOM | 2928 | CB | ILE | 358 | 2.869 | 12.295 | 38.630 | 1.00 | 0.94 | 1SG | 2929 |
| ATOM | 2929 | CG2 | ILE | 358 | 1.945 | 11.490 | 37.701 | 1.00 | 0.94 | 1SG | 2930 |
| ATOM | 2930 | CG1 | ILE | 358 | 4.168 | 11.567 | 38.996 | 1.00 | 0.94 | 1SG | 2931 |
| ATOM | 2931 | CD1 | ILE | 358 | 3.936 | 10.227 | 39.673 | 1.00 | 0.94 | 1SG | 2932 |
| ATOM | 2932 | C | ILE | 358 | 3.961 | 13.192 | 36.737 | 1.00 | 0.94 | 1SG | 2933 |
| ATOM | 2933 | O | ILE | 358 | 3.331 | 12.823 | 35.746 | 1.00 | 0.94 | 1SG | 2934 |
| ATOM | 2934 | N | LYS | 359 | 5.301 | 13.256 | 36.745 | 1.00 | 1.15 | 1SG | 2935 |
| ATOM | 2935 | CA | LYS | 359 | 5.955 | 12.856 | 35.538 | 1.00 | 1.15 | 1SG | 2936 |
| ATOM | 2936 | CB | LYS | 359 | 7.333 | 13.492 | 35.273 | 1.00 | 1.15 | 1SG | 2937 |
| ATOM | 2937 | CG | LYS | 359 | 8.540 | 12.600 | 35.595 | 1.00 | 1.15 | 1SG | 2938 |
| ATOM | 2938 | CD | LYS | 359 | 8.847 | 11.564 | 34.511 | 1.00 | 1.15 | 1SG | 2939 |
| ATOM | 2939 | CE | LYS | 359 | 9.356 | 12.188 | 33.210 | 1.00 | 1.15 | 1SG | 2940 |
| ATOM | 2940 | NZ | LYS | 359 | 9.634 | 11.131 | 32.212 | 1.00 | 1.15 | 1SG | 2941 |
| ATOM | 2941 | C | LYS | 359 | 6.165 | 11.415 | 35.723 | 1.00 | 1.15 | 1SG | 2942 |
| ATOM | 2942 | O | LYS | 359 | 6.661 | 10.981 | 36.766 | 1.00 | 1.15 | 1SG | 2943 |
| ATOM | 2943 | N | ASP | 360 | 5.778 | 10.627 | 34.714 | 1.00 | 1.09 | 1SG | 2944 |
| ATOM | 2944 | CA | ASP | 360 | 5.883 | 9.249 | 35.009 | 1.00 | 1.09 | 1SG | 2945 |
| ATOM | 2945 | CB | ASP | 360 | 4.844 | 8.838 | 36.068 | 1.00 | 1.09 | 1SG | 2946 |
| ATOM | 2946 | CG | ASP | 360 | 3.447 | 9.122 | 35.548 | 1.00 | 1.09 | 1SG | 2947 |
| ATOM | 2947 | OD1 | ASP | 360 | 3.319 | 9.698 | 34.436 | 1.00 | 1.09 | 1SG | 2948 |
| ATOM | 2948 | OD2 | ASP | 360 | 2.483 | 8.772 | 36.275 | 1.00 | 1.09 | 1SG | 2949 |
| ATOM | 2949 | C | ASP | 360 | 5.665 | 8.435 | 33.787 | 1.00 | 1.09 | 1SG | 2950 |
| ATOM | 2950 | O | ASP | 360 | 5.719 | 8.912 | 32.655 | 1.00 | 1.09 | 1SG | 2951 |
| ATOM | 2951 | N | GLY | 361 | 5.431 | 7.142 | 34.050 | 1.00 | 0.86 | 1SG | 2952 |
| ATOM | 2952 | CA | GLY | 361 | 5.160 | 6.117 | 33.101 | 1.00 | 0.86 | 1SG | 2953 |
| ATOM | 2953 | C | GLY | 361 | 4.127 | 5.329 | 33.817 | 1.00 | 0.86 | 1SG | 2954 |
| ATOM | 2954 | O | GLY | 361 | 3.333 | 5.907 | 34.559 | 1.00 | 0.86 | 1SG | 2955 |
| ATOM | 2955 | N | HIS | 362 | 4.073 | 4.003 | 33.616 | 1.00 | 0.86 | 1SG | 2956 |
| ATOM | 2956 | CA | HIS | 362 | 3.177 | 3.315 | 34.487 | 1.00 | 0.86 | 1SG | 2957 |
| ATOM | 2957 | ND1 | HIS | 362 | 2.564 | 0.872 | 36.580 | 1.00 | 0.86 | 1SG | 2958 |
| ATOM | 2958 | CG | HIS | 362 | 2.194 | 1.160 | 35.286 | 1.00 | 0.86 | 1SG | 2959 |
| ATOM | 2959 | CB | HIS | 362 | 3.111 | 1.796 | 34.282 | 1.00 | 0.86 | 1SG | 2960 |
| ATOM | 2960 | NE2 | HIS | 362 | 0.438 | 0.256 | 36.377 | 1.00 | 0.86 | 1SG | 2961 |
| ATOM | 2961 | CD2 | HIS | 362 | 0.893 | 0.776 | 35.178 | 1.00 | 0.86 | 1SG | 2962 |
| ATOM | 2962 | CE1 | HIS | 362 | 1.476 | 0.334 | 37.188 | 1.00 | 0.86 | 1SG | 2963 |
| ATOM | 2963 | C | HIS | 362 | 3.774 | 3.526 | 35.830 | 1.00 | 0.86 | 1SG | 2964 |
| ATOM | 2964 | O | HIS | 362 | 3.080 | 3.769 | 36.815 | 1.00 | 0.86 | 1SG | 2965 |
| ATOM | 2965 | N | TRP | 363 | 5.116 | 3.449 | 35.875 | 1.00 | 1.03 | 1SG | 2966 |
| ATOM | 2966 | CA | TRP | 363 | 5.820 | 3.644 | 37.098 | 1.00 | 1.03 | 1SG | 2967 |
| ATOM | 2967 | CB | TRP | 363 | 6.586 | 2.385 | 37.535 | 1.00 | 1.03 | 1SG | 2968 |
| ATOM | 2968 | CG | TRP | 363 | 7.074 | 1.566 | 36.365 | 1.00 | 1.03 | 1SG | 2969 |
| ATOM | 2969 | CD2 | TRP | 363 | 8.022 | 1.993 | 35.375 | 1.00 | 1.03 | 1SG | 2970 |
| ATOM | 2970 | CD1 | TRP | 363 | 6.640 | 0.333 | 35.974 | 1.00 | 1.03 | 1SG | 2971 |
| ATOM | 2971 | NE1 | TRP | 363 | 7.264 | −0.038 | 34.809 | 1.00 | 1.03 | 1SG | 2972 |
| ATOM | 2972 | CE2 | TRP | 363 | 8.113 | 0.977 | 34.427 | 1.00 | 1.03 | 1SG | 2973 |
| ATOM | 2973 | CE3 | TRP | 363 | 8.748 | 3.143 | 35.251 | 1.00 | 1.03 | 1SG | 2974 |
| ATOM | 2974 | CZ2 | TRP | 363 | 8.930 | 1.098 | 33.344 | 1.00 | 1.03 | 1SG | 2975 |
| ATOM | 2975 | CZ3 | TRP | 363 | 9.585 | 3.258 | 34.167 | 1.00 | 1.03 | 1SG | 2976 |
| ATOM | 2976 | CH2 | TRP | 363 | 9.673 | 2.253 | 33.230 | 1.00 | 1.03 | 1SG | 2977 |
| ATOM | 2977 | C | TRP | 363 | 6.788 | 4.782 | 36.999 | 1.00 | 1.03 | 1SG | 2978 |
| ATOM | 2978 | O | TRP | 363 | 7.944 | 4.632 | 37.393 | 1.00 | 1.03 | 1SG | 2979 |
| ATOM | 2979 | N | PHE | 364 | 6.348 | 5.966 | 36.508 | 1.00 | 0.94 | 1SG | 2980 |
| ATOM | 2980 | CA | PHE | 364 | 7.163 | 7.136 | 36.591 | 1.00 | 0.94 | 1SG | 2981 |
| ATOM | 2981 | CB | PHE | 364 | 7.795 | 7.297 | 37.981 | 1.00 | 0.94 | 1SG | 2982 |
| ATOM | 2982 | CG | PHE | 364 | 6.713 | 6.961 | 38.956 | 1.00 | 0.94 | 1SG | 2983 |
| ATOM | 2983 | CD1 | PHE | 364 | 5.591 | 7.737 | 39.090 | 1.00 | 0.94 | 1SG | 2984 |
| ATOM | 2984 | CD2 | PHE | 364 | 6.822 | 5.830 | 39.734 | 1.00 | 0.94 | 1SG | 2985 |
| ATOM | 2985 | CE1 | PHE | 364 | 4.621 | 7.385 | 40.001 | 1.00 | 0.94 | 1SG | 2986 |
| ATOM | 2986 | CE2 | PHE | 364 | 5.856 | 5.474 | 40.643 | 1.00 | 0.94 | 1SG | 2987 |
| ATOM | 2987 | CZ | PHE | 364 | 4.743 | 6.261 | 40.779 | 1.00 | 0.94 | 1SG | 2988 |
| ATOM | 2988 | C | PHE | 364 | 8.210 | 6.980 | 35.556 | 1.00 | 0.94 | 1SG | 2989 |
| ATOM | 2989 | O | PHE | 364 | 7.948 | 6.575 | 34.426 | 1.00 | 0.94 | 1SG | 2990 |
| ATOM | 2990 | N | ALA | 365 | 9.449 | 7.311 | 35.922 | 1.00 | 0.53 | 1SG | 2991 |
| ATOM | 2991 | CA | ALA | 365 | 10.498 | 7.093 | 34.988 | 1.00 | 0.53 | 1SG | 2992 |
| ATOM | 2992 | CB | ALA | 365 | 11.307 | 8.359 | 34.660 | 1.00 | 0.53 | 1SG | 2993 |
| ATOM | 2993 | C | ALA | 365 | 11.429 | 6.161 | 35.673 | 1.00 | 0.53 | 1SG | 2994 |
| ATOM | 2994 | O | ALA | 365 | 11.439 | 6.065 | 36.899 | 1.00 | 0.53 | 1SG | 2995 |
| ATOM | 2995 | N | ASN | 366 | 12.217 | 5.418 | 34.885 | 1.00 | 0.54 | 1SG | 2996 |
| ATOM | 2996 | CA | ASN | 366 | 13.163 | 4.540 | 35.492 | 1.00 | 0.54 | 1SG | 2997 |
| ATOM | 2997 | CB | ASN | 366 | 13.365 | 3.240 | 34.689 | 1.00 | 0.54 | 1SG | 2998 |
| ATOM | 2998 | CG | ASN | 366 | 14.043 | 2.205 | 35.563 | 1.00 | 0.54 | 1SG | 2999 |
| ATOM | 2999 | OD1 | ASN | 366 | 14.702 | 2.552 | 36.539 | 1.00 | 0.54 | 1SG | 3000 |
| ATOM | 3000 | ND2 | ASN | 366 | 13.881 | 0.902 | 35.209 | 1.00 | 0.54 | 1SG | 3001 |
| ATOM | 3001 | C | ASN | 366 | 14.451 | 5.299 | 35.490 | 1.00 | 0.54 | 1SG | 3002 |
| ATOM | 3002 | O | ASN | 366 | 14.996 | 5.609 | 34.433 | 1.00 | 0.54 | 1SG | 3003 |
| ATOM | 3003 | N | LYS | 367 | 14.954 | 5.660 | 36.684 | 1.00 | 0.78 | 1SG | 3004 |
| ATOM | 3004 | CA | LYS | 367 | 16.188 | 6.388 | 36.738 | 1.00 | 0.78 | 1SG | 3005 |
| ATOM | 3005 | CB | LYS | 367 | 16.081 | 7.690 | 37.551 | 1.00 | 0.78 | 1SG | 3006 |
| ATOM | 3006 | CG | LYS | 367 | 17.170 | 8.714 | 37.228 | 1.00 | 0.78 | 1SG | 3007 |
| ATOM | 3007 | CD | LYS | 367 | 18.588 | 8.217 | 37.496 | 1.00 | 0.78 | 1SG | 3008 |
| ATOM | 3008 | CE | LYS | 367 | 19.673 | 9.215 | 37.088 | 1.00 | 0.78 | 1SG | 3009 |
| ATOM | 3009 | NZ | LYS | 367 | 19.642 | 10.392 | 37.985 | 1.00 | 0.78 | 1SG | 3010 |
| ATOM | 3010 | C | LYS | 367 | 17.145 | 5.479 | 37.427 | 1.00 | 0.78 | 1SG | 3011 |
| ATOM | 3011 | O | LYS | 367 | 16.936 | 5.118 | 38.585 | 1.00 | 0.78 | 1SG | 3012 |
| ATOM | 3012 | N | PHE | 368 | 18.239 | 5.086 | 36.738 | 1.00 | 0.66 | 1SG | 3013 |
| ATOM | 3013 | CA | PHE | 368 | 19.108 | 4.065 | 37.260 | 1.00 | 0.66 | 1SG | 3014 |
| ATOM | 3014 | CB | PHE | 368 | 19.991 | 4.498 | 38.448 | 1.00 | 0.66 | 1SG | 3015 |
| ATOM | 3015 | CG | PHE | 368 | 21.094 | 5.347 | 37.911 | 1.00 | 0.66 | 1SG | 3016 |
| ATOM | 3016 | CD1 | PHE | 368 | 22.177 | 4.767 | 37.289 | 1.00 | 0.66 | 1SG | 3017 |
| ATOM | 3017 | CD2 | PHE | 368 | 21.062 | 6.716 | 38.038 | 1.00 | 0.66 | 1SG | 3018 |
| ATOM | 3018 | CE1 | PHE | 368 | 23.202 | 5.540 | 36.793 | 1.00 | 0.66 | 1SG | 3019 |
| ATOM | 3019 | CE2 | PHE | 368 | 22.082 | 7.495 | 37.544 | 1.00 | 0.66 | 1SG | 3020 |
| ATOM | 3020 | CZ | PHE | 368 | 23.155 | 6.907 | 36.919 | 1.00 | 0.66 | 1SG | 3021 |
| ATOM | 3021 | C | PHE | 368 | 18.258 | 2.897 | 37.666 | 1.00 | 0.66 | 1SG | 3022 |
| ATOM | 3022 | O | PHE | 368 | 17.110 | 2.781 | 37.260 | 1.00 | 0.66 | 1SG | 3023 |
| ATOM | 3023 | N | ASP | 369 | 18.791 | 1.969 | 38.469 | 1.00 | 0.54 | 1SG | 3024 |
| ATOM | 3024 | CA | ASP | 369 | 18.039 | 0.802 | 38.839 | 1.00 | 0.54 | 1SG | 3025 |
| ATOM | 3025 | CB | ASP | 369 | 18.792 | −0.076 | 39.857 | 1.00 | 0.54 | 1SG | 3026 |
| ATOM | 3026 | CG | ASP | 369 | 18.106 | −1.433 | 39.939 | 1.00 | 0.54 | 1SG | 3027 |
| ATOM | 3027 | OD1 | ASP | 369 | 17.148 | −1.668 | 39.156 | 1.00 | 0.54 | 1SG | 3028 |
| ATOM | 3028 | OD2 | ASP | 369 | 18.538 | −2.258 | 40.788 | 1.00 | 0.54 | 1SG | 3029 |
| ATOM | 3029 | C | ASP | 369 | 16.744 | 1.225 | 39.469 | 1.00 | 0.54 | 1SG | 3030 |
| ATOM | 3030 | O | ASP | 369 | 15.794 | 0.447 | 39.520 | 1.00 | 0.54 | 1SG | 3031 |
| ATOM | 3031 | N | SER | 370 | 16.671 | 2.478 | 39.967 | 1.00 | 0.65 | 1SG | 3032 |
| ATOM | 3032 | CA | SER | 370 | 15.473 | 2.968 | 40.596 | 1.00 | 0.65 | 1SG | 3033 |
| ATOM | 3033 | CB | SER | 370 | 15.650 | 4.341 | 41.264 | 1.00 | 0.65 | 1SG | 3034 |
| ATOM | 3034 | OG | SER | 370 | 14.432 | 4.756 | 41.863 | 1.00 | 0.65 | 1SG | 3035 |
| ATOM | 3035 | C | SER | 370 | 14.447 | 3.109 | 39.530 | 1.00 | 0.65 | 1SG | 3036 |
| ATOM | 3036 | O | SER | 370 | 14.283 | 4.165 | 38.923 | 1.00 | 0.65 | 1SG | 3037 |
| ATOM | 3037 | N | LYS | 371 | 13.711 | 2.011 | 39.315 | 1.00 | 0.66 | 1SG | 3038 |
| ATOM | 3038 | CA | LYS | 371 | 12.712 | 1.923 | 38.310 | 1.00 | 0.66 | 1SG | 3039 |
| ATOM | 3039 | CB | LYS | 371 | 12.156 | 0.489 | 38.201 | 1.00 | 0.66 | 1SG | 3040 |
| ATOM | 3040 | CG | LYS | 371 | 11.074 | 0.287 | 37.144 | 1.00 | 0.66 | 1SG | 3041 |
| ATOM | 3041 | CD | LYS | 371 | 9.735 | 0.918 | 37.518 | 1.00 | 0.66 | 1SG | 3042 |
| ATOM | 3042 | CE | LYS | 371 | 9.064 | 0.212 | 38.697 | 1.00 | 0.66 | 1SG | 3043 |
| ATOM | 3043 | NZ | LYS | 371 | 8.954 | −1.239 | 38.423 | 1.00 | 0.66 | 1SG | 3044 |
| ATOM | 3044 | C | LYS | 371 | 11.629 | 2.874 | 38.664 | 1.00 | 0.66 | 1SG | 3045 |
| ATOM | 3045 | O | LYS | 371 | 10.941 | 3.398 | 37.789 | 1.00 | 0.66 | 1SG | 3046 |
| ATOM | 3046 | N | VAL | 372 | 11.453 | 3.152 | 39.970 | 1.00 | 0.47 | 1SG | 3047 |
| ATOM | 3047 | CA | VAL | 372 | 10.338 | 3.982 | 40.291 | 1.00 | 0.47 | 1SG | 3048 |
| ATOM | 3048 | CB | VAL | 372 | 9.527 | 3.423 | 41.420 | 1.00 | 0.47 | 1SG | 3049 |
| ATOM | 3049 | CG1 | VAL | 372 | 8.416 | 4.421 | 41.761 | 1.00 | 0.47 | 1SG | 3050 |
| ATOM | 3050 | CG2 | VAL | 372 | 9.019 | 2.029 | 41.020 | 1.00 | 0.47 | 1SG | 3051 |
| ATOM | 3051 | C | VAL | 372 | 10.786 | 5.344 | 40.717 | 1.00 | 0.47 | 1SG | 3052 |
| ATOM | 3052 | O | VAL | 372 | 11.420 | 5.501 | 41.759 | 1.00 | 0.47 | 1SG | 3053 |
| ATOM | 3053 | N | ASP | 373 | 10.445 | 6.378 | 39.916 | 1.00 | 0.41 | 1SG | 3054 |
| ATOM | 3054 | CA | ASP | 373 | 10.803 | 7.717 | 40.287 | 1.00 | 0.41 | 1SG | 3055 |
| ATOM | 3055 | CB | ASP | 373 | 12.045 | 8.227 | 39.538 | 1.00 | 0.41 | 1SG | 3056 |
| ATOM | 3056 | CG | ASP | 373 | 12.339 | 9.647 | 39.997 | 1.00 | 0.41 | 1SG | 3057 |
| ATOM | 3057 | OD1 | ASP | 373 | 11.757 | 10.076 | 41.029 | 1.00 | 0.41 | 1SG | 3058 |
| ATOM | 3058 | OD2 | ASP | 373 | 13.159 | 10.324 | 39.320 | 1.00 | 0.41 | 1SG | 3059 |
| ATOM | 3059 | C | ASP | 373 | 9.696 | 8.657 | 39.910 | 1.00 | 0.41 | 1SG | 3060 |
| ATOM | 3060 | O | ASP | 373 | 9.663 | 9.131 | 38.776 | 1.00 | 0.41 | 1SG | 3061 |
| ATOM | 3061 | N | PRO | 374 | 8.770 | 8.931 | 40.791 | 1.00 | 0.57 | 1SG | 3062 |
| ATOM | 3062 | CA | PRO | 374 | 7.790 | 9.930 | 40.456 | 1.00 | 0.57 | 1SG | 3063 |
| ATOM | 3063 | CD | PRO | 374 | 8.152 | 7.848 | 41.532 | 1.00 | 0.57 | 1SG | 3064 |
| ATOM | 3064 | CB | PRO | 374 | 6.527 | 9.609 | 41.257 | 1.00 | 0.57 | 1SG | 3065 |
| ATOM | 3065 | CG | PRO | 374 | 6.957 | 8.505 | 42.237 | 1.00 | 0.57 | 1SG | 3066 |
| ATOM | 3066 | C | PRO | 374 | 8.253 | 11.326 | 40.716 | 1.00 | 0.57 | 1SG | 3067 |
| ATOM | 3067 | O | PRO | 374 | 8.603 | 11.624 | 41.856 | 1.00 | 0.57 | 1SG | 3068 |
| ATOM | 3068 | N | ILE | 375 | 8.219 | 12.223 | 39.713 | 1.00 | 0.80 | 1SG | 3069 |
| ATOM | 3069 | CA | ILE | 375 | 8.477 | 13.585 | 40.071 | 1.00 | 0.80 | 1SG | 3070 |
| ATOM | 3070 | CB | ILE | 375 | 9.271 | 14.394 | 39.066 | 1.00 | 0.80 | 1SG | 3071 |
| ATOM | 3071 | CG2 | ILE | 375 | 8.465 | 14.569 | 37.775 | 1.00 | 0.80 | 1SG | 3072 |
| ATOM | 3072 | CG1 | ILE | 375 | 9.698 | 15.740 | 39.674 | 1.00 | 0.80 | 1SG | 3073 |
| ATOM | 3073 | CD1 | ILE | 375 | 8.526 | 16.683 | 39.943 | 1.00 | 0.80 | 1SG | 3074 |
| ATOM | 3074 | C | ILE | 375 | 7.101 | 14.134 | 40.254 | 1.00 | 0.80 | 1SG | 3075 |
| ATOM | 3075 | O | ILE | 375 | 6.279 | 14.108 | 39.339 | 1.00 | 0.80 | 1SG | 3076 |
| ATOM | 3076 | N | LEU | 376 | 6.780 | 14.613 | 41.471 | 1.00 | 0.88 | 1SG | 3077 |
| ATOM | 3077 | CA | LEU | 376 | 5.413 | 15.002 | 41.640 | 1.00 | 0.88 | 1SG | 3078 |
| ATOM | 3078 | CB | LEU | 376 | 4.582 | 13.892 | 42.320 | 1.00 | 0.88 | 1SG | 3079 |
| ATOM | 3079 | CG | LEU | 376 | 3.052 | 14.017 | 42.161 | 1.00 | 0.88 | 1SG | 3080 |
| ATOM | 3080 | CD2 | LEU | 376 | 2.732 | 14.461 | 40.737 | 1.00 | 0.88 | 1SG | 3081 |
| ATOM | 3081 | CD1 | LEU | 376 | 2.375 | 14.934 | 43.181 | 1.00 | 0.88 | 1SG | 3082 |
| ATOM | 3082 | C | LEU | 376 | 5.349 | 16.265 | 42.440 | 1.00 | 0.88 | 1SG | 3083 |
| ATOM | 3083 | O | LEU | 376 | 6.317 | 16.657 | 43.091 | 1.00 | 0.88 | 1SG | 3084 |
| ATOM | 3084 | N | ILE | 377 | 4.186 | 16.944 | 42.372 | 1.00 | 0.85 | 1SG | 3085 |
| ATOM | 3085 | CA | ILE | 377 | 3.949 | 18.189 | 43.046 | 1.00 | 0.85 | 1SG | 3086 |
| ATOM | 3086 | CB | ILE | 377 | 2.843 | 18.969 | 42.368 | 1.00 | 0.85 | 1SG | 3087 |
| ATOM | 3087 | CG2 | ILE | 377 | 2.721 | 20.374 | 42.989 | 1.00 | 0.85 | 1SG | 3088 |
| ATOM | 3088 | CG1 | ILE | 377 | 3.156 | 19.074 | 40.868 | 1.00 | 0.85 | 1SG | 3089 |
| ATOM | 3089 | CD1 | ILE | 377 | 4.451 | 19.830 | 40.574 | 1.00 | 0.85 | 1SG | 3090 |
| ATOM | 3090 | C | ILE | 377 | 3.546 | 17.839 | 44.455 | 1.00 | 0.85 | 1SG | 3091 |
| ATOM | 3091 | O | ILE | 377 | 3.608 | 16.681 | 44.859 | 1.00 | 0.85 | 1SG | 3092 |
| ATOM | 3092 | N | LYS | 378 | 3.169 | 18.830 | 45.282 | 1.00 | 0.86 | 1SG | 3093 |
| ATOM | 3093 | CA | LYS | 378 | 2.756 | 18.497 | 46.611 | 1.00 | 0.86 | 1SG | 3094 |
| ATOM | 3094 | CB | LYS | 378 | 2.561 | 19.722 | 47.522 | 1.00 | 0.86 | 1SG | 3095 |
| ATOM | 3095 | CG | LYS | 378 | 3.855 | 20.490 | 47.798 | 1.00 | 0.86 | 1SG | 3096 |
| ATOM | 3096 | CD | LYS | 378 | 4.432 | 21.175 | 46.558 | 1.00 | 0.86 | 1SG | 3097 |
| ATOM | 3097 | CE | LYS | 378 | 3.588 | 22.349 | 46.060 | 1.00 | 0.86 | 1SG | 3098 |
| ATOM | 3098 | NZ | LYS | 378 | 2.283 | 21.860 | 45.563 | 1.00 | 0.86 | 1SG | 3099 |
| ATOM | 3099 | C | LYS | 378 | 1.440 | 17.808 | 46.488 | 1.00 | 0.86 | 1SG | 3100 |
| ATOM | 3100 | O | LYS | 378 | 0.712 | 18.013 | 45.517 | 1.00 | 0.86 | 1SG | 3101 |
| ATOM | 3101 | N | CYS | 379 | 1.107 | 16.948 | 47.470 | 1.00 | 0.84 | 1SG | 3102 |
| ATOM | 3102 | CA | CYS | 379 | −0.136 | 16.239 | 47.396 | 1.00 | 0.84 | 1SG | 3103 |
| ATOM | 3103 | CB | CYS | 379 | −0.174 | 14.986 | 48.289 | 1.00 | 0.84 | 1SG | 3104 |
| ATOM | 3104 | SG | CYS | 379 | 0.095 | 15.359 | 50.049 | 1.00 | 0.84 | 1SG | 3105 |
| ATOM | 3105 | C | CYS | 379 | −1.212 | 17.168 | 47.840 | 1.00 | 0.84 | 1SG | 3106 |
| ATOM | 3106 | O | CYS | 379 | −1.973 | 16.869 | 48.761 | 1.00 | 0.84 | 1SG | 3107 |
| ATOM | 3107 | N | LEU | 380 | −1.305 | 18.322 | 47.164 | 1.00 | 0.81 | 1SG | 3108 |
| ATOM | 3108 | CA | LEU | 380 | −2.309 | 19.285 | 47.478 | 1.00 | 0.81 | 1SG | 3109 |
| ATOM | 3109 | CB | LEU | 380 | −1.770 | 20.516 | 48.227 | 1.00 | 0.81 | 1SG | 3110 |
| ATOM | 3110 | CG | LEU | 380 | −2.864 | 21.551 | 48.555 | 1.00 | 0.81 | 1SG | 3111 |
| ATOM | 3111 | CD2 | LEU | 380 | −2.253 | 22.863 | 49.075 | 1.00 | 0.81 | 1SG | 3112 |
| ATOM | 3112 | CD1 | LEU | 380 | −3.921 | 20.964 | 49.513 | 1.00 | 0.81 | 1SG | 3113 |
| ATOM | 3113 | C | LEU | 380 | −2.804 | 19.756 | 46.159 | 1.00 | 0.81 | 1SG | 3114 |
| ATOM | 3114 | O | LEU | 380 | −3.855 | 19.332 | 45.680 | 1.00 | 0.81 | 1SG | 3115 |
| ATOM | 3115 | N | ALA | 381 | −2.030 | 20.657 | 45.530 | 1.00 | 0.83 | 1SG | 3116 |
| ATOM | 3116 | CA | ALA | 381 | −2.414 | 21.160 | 44.253 | 1.00 | 0.83 | 1SG | 3117 |
| ATOM | 3117 | CB | ALA | 381 | −1.555 | 22.342 | 43.773 | 1.00 | 0.83 | 1SG | 3118 |
| ATOM | 3118 | C | ALA | 381 | −2.257 | 20.061 | 43.270 | 1.00 | 0.83 | 1SG | 3119 |
| ATOM | 3119 | O | ALA | 381 | −1.470 | 19.138 | 43.467 | 1.00 | 0.83 | 1SG | 3120 |
| ATOM | 3120 | N | GLU | 382 | −3.042 | 20.120 | 42.182 | 1.00 | 1.00 | 1SG | 3121 |
| ATOM | 3121 | CA | GLU | 382 | −2.918 | 19.096 | 41.195 | 1.00 | 1.00 | 1SG | 3122 |
| ATOM | 3122 | CB | GLU | 382 | −3.960 | 17.971 | 41.327 | 1.00 | 1.00 | 1SG | 3123 |
| ATOM | 3123 | CG | GLU | 382 | −5.409 | 18.436 | 41.195 | 1.00 | 1.00 | 1SG | 3124 |
| ATOM | 3124 | CD | GLU | 382 | −5.863 | 18.970 | 42.544 | 1.00 | 1.00 | 1SG | 3125 |
| ATOM | 3125 | OE1 | GLU | 382 | −5.098 | 18.801 | 43.531 | 1.00 | 1.00 | 1SG | 3126 |
| ATOM | 3126 | OE2 | GLU | 382 | −6.981 | 19.547 | 42.609 | 1.00 | 1.00 | 1SG | 3127 |
| ATOM | 3127 | C | GLU | 382 | −3.114 | 19.733 | 39.864 | 1.00 | 1.00 | 1SG | 3128 |
| ATOM | 3128 | O | GLU | 382 | −3.172 | 20.954 | 39.740 | 1.00 | 1.00 | 1SG | 3129 |
| ATOM | 3129 | N | LYS | 383 | −3.204 | 18.887 | 38.826 | 1.00 | 1.10 | 1SG | 3130 |
| ATOM | 3130 | CA | LYS | 383 | −3.428 | 19.300 | 37.478 | 1.00 | 1.10 | 1SG | 3131 |
| ATOM | 3131 | CB | LYS | 383 | −3.408 | 18.146 | 36.464 | 1.00 | 1.10 | 1SG | 3132 |
| ATOM | 3132 | CG | LYS | 383 | −3.656 | 18.618 | 35.030 | 1.00 | 1.10 | 1SG | 3133 |
| ATOM | 3133 | CD | LYS | 383 | −2.573 | 19.550 | 34.483 | 1.00 | 1.10 | 1SG | 3134 |
| ATOM | 3134 | CE | LYS | 383 | −2.868 | 20.055 | 33.069 | 1.00 | 1.10 | 1SG | 3135 |
| ATOM | 3135 | NZ | LYS | 383 | −1.887 | 21.094 | 32.686 | 1.00 | 1.10 | 1SG | 3136 |
| ATOM | 3136 | C | LYS | 383 | −4.780 | 19.906 | 37.449 | 1.00 | 1.10 | 1SG | 3137 |
| ATOM | 3137 | O | LYS | 383 | −5.111 | 20.637 | 36.516 | 1.00 | 1.10 | 1SG | 3138 |
| ATOM | 3138 | N | LEU | 384 | −5.595 | 19.591 | 38.478 | 1.00 | 0.93 | 1SG | 3139 |
| ATOM | 3139 | CA | LEU | 384 | −6.917 | 20.137 | 38.545 | 1.00 | 0.93 | 1SG | 3140 |
| ATOM | 3140 | CB | LEU | 384 | −7.658 | 19.781 | 39.851 | 1.00 | 0.93 | 1SG | 3141 |
| ATOM | 3141 | CG | LEU | 384 | −9.095 | 20.333 | 39.975 | 1.00 | 0.93 | 1SG | 3142 |
| ATOM | 3142 | CD2 | LEU | 384 | −9.979 | 19.845 | 38.811 | 1.00 | 0.93 | 1SG | 3143 |
| ATOM | 3143 | CD1 | LEU | 384 | −9.130 | 21.856 | 40.151 | 1.00 | 0.93 | 1SG | 3144 |
| ATOM | 3144 | C | LEU | 384 | −6.707 | 21.607 | 38.503 | 1.00 | 0.93 | 1SG | 3145 |
| ATOM | 3145 | O | LEU | 384 | −7.426 | 22.338 | 37.822 | 1.00 | 0.93 | 1SG | 3146 |
| ATOM | 3146 | N | GLU | 385 | −5.680 | 22.079 | 39.230 | 1.00 | 0.67 | 1SG | 3147 |
| ATOM | 3147 | CA | GLU | 385 | −5.361 | 23.464 | 39.135 | 1.00 | 0.67 | 1SG | 3148 |
| ATOM | 3148 | CB | GLU | 385 | −5.118 | 24.135 | 40.497 | 1.00 | 0.67 | 1SG | 3149 |
| ATOM | 3149 | CG | GLU | 385 | −6.369 | 24.215 | 41.374 | 1.00 | 0.67 | 1SG | 3150 |
| ATOM | 3150 | CD | GLU | 385 | −6.600 | 22.847 | 42.002 | 1.00 | 0.67 | 1SG | 3151 |
| ATOM | 3151 | OE1 | GLU | 385 | −5.696 | 21.978 | 41.877 | 1.00 | 0.67 | 1SG | 3152 |
| ATOM | 3152 | OE2 | GLU | 385 | −7.682 | 22.655 | 42.619 | 1.00 | 0.67 | 1SG | 3153 |
| ATOM | 3153 | C | GLU | 385 | −4.083 | 23.533 | 38.369 | 1.00 | 0.67 | 1SG | 3154 |
| ATOM | 3154 | O | GLU | 385 | −3.006 | 23.351 | 38.934 | 1.00 | 0.67 | 1SG | 3155 |
| ATOM | 3155 | N | GLU | 386 | −4.175 | 23.775 | 37.047 | 1.00 | 0.48 | 1SG | 3156 |
| ATOM | 3156 | CA | GLU | 386 | −2.996 | 23.887 | 36.241 | 1.00 | 0.48 | 1SG | 3157 |
| ATOM | 3157 | CB | GLU | 386 | −3.305 | 23.948 | 34.737 | 1.00 | 0.48 | 1SG | 3158 |
| ATOM | 3158 | CG | GLU | 386 | −3.973 | 22.691 | 34.180 | 1.00 | 0.48 | 1SG | 3159 |
| ATOM | 3159 | CD | GLU | 386 | −4.408 | 23.006 | 32.755 | 1.00 | 0.48 | 1SG | 3160 |
| ATOM | 3160 | OE1 | GLU | 386 | −3.536 | 23.429 | 31.949 | 1.00 | 0.48 | 1SG | 3161 |
| ATOM | 3161 | OE2 | GLU | 386 | −5.622 | 22.841 | 32.457 | 1.00 | 0.48 | 1SG | 3162 |
| ATOM | 3162 | C | GLU | 386 | −2.411 | 25.210 | 36.593 | 1.00 | 0.48 | 1SG | 3163 |
| ATOM | 3163 | O | GLU | 386 | −2.578 | 26.184 | 35.861 | 1.00 | 0.48 | 1SG | 3164 |
| ATOM | 3164 | N | GLN | 387 | −1.718 | 25.284 | 37.741 | 1.00 | 0.38 | 1SG | 3165 |
| ATOM | 3165 | CA | GLN | 387 | −1.174 | 26.539 | 38.138 | 1.00 | 0.38 | 1SG | 3166 |
| ATOM | 3166 | CB | GLN | 387 | −1.912 | 27.154 | 39.341 | 1.00 | 0.38 | 1SG | 3167 |
| ATOM | 3167 | CG | GLN | 387 | −3.403 | 27.406 | 39.097 | 1.00 | 0.38 | 1SG | 3168 |
| ATOM | 3168 | CD | GLN | 387 | −3.997 | 28.015 | 40.358 | 1.00 | 0.38 | 1SG | 3169 |
| ATOM | 3169 | OE1 | GLN | 387 | −3.304 | 28.197 | 41.357 | 1.00 | 0.38 | 1SG | 3170 |
| ATOM | 3170 | NE2 | GLN | 387 | −5.318 | 28.336 | 40.316 | 1.00 | 0.38 | 1SG | 3171 |
| ATOM | 3171 | C | GLN | 387 | 0.225 | 26.303 | 38.592 | 1.00 | 0.38 | 1SG | 3172 |
| ATOM | 3172 | O | GLN | 387 | 0.478 | 26.263 | 39.796 | 1.00 | 0.38 | 1SG | 3173 |
| ATOM | 3173 | N | GLN | 388 | 1.176 | 26.114 | 37.654 | 1.00 | 0.53 | 1SG | 3174 |
| ATOM | 3174 | CA | GLN | 388 | 2.520 | 26.010 | 38.135 | 1.00 | 0.53 | 1SG | 3175 |
| ATOM | 3175 | CB | GLN | 388 | 3.559 | 25.873 | 37.017 | 1.00 | 0.53 | 1SG | 3176 |
| ATOM | 3176 | CG | GLN | 388 | 3.486 | 24.579 | 36.219 | 1.00 | 0.53 | 1SG | 3177 |
| ATOM | 3177 | CD | GLN | 388 | 4.559 | 24.690 | 35.150 | 1.00 | 0.53 | 1SG | 3178 |
| ATOM | 3178 | OE1 | GLN | 388 | 5.752 | 24.694 | 35.450 | 1.00 | 0.53 | 1SG | 3179 |
| ATOM | 3179 | NE2 | GLN | 388 | 4.121 | 24.813 | 33.869 | 1.00 | 0.53 | 1SG | 3180 |
| ATOM | 3180 | C | GLN | 388 | 2.766 | 27.338 | 38.749 | 1.00 | 0.53 | 1SG | 3181 |
| ATOM | 3181 | O | GLN | 388 | 2.823 | 27.480 | 39.969 | 1.00 | 0.53 | 1SG | 3182 |
| ATOM | 3182 | N | ARG | 389 | 2.902 | 28.362 | 37.886 | 1.00 | 0.74 | 1SG | 3183 |
| ATOM | 3183 | CA | ARG | 389 | 2.937 | 29.694 | 38.394 | 1.00 | 0.74 | 1SG | 3184 |
| ATOM | 3184 | CB | ARG | 389 | 3.339 | 30.733 | 37.336 | 1.00 | 0.74 | 1SG | 3185 |
| ATOM | 3185 | CG | ARG | 389 | 3.379 | 32.165 | 37.872 | 1.00 | 0.74 | 1SG | 3186 |
| ATOM | 3186 | CD | ARG | 389 | 3.776 | 33.202 | 36.820 | 1.00 | 0.74 | 1SG | 3187 |
| ATOM | 3187 | NE | ARG | 389 | 5.161 | 32.876 | 36.376 | 1.00 | 0.74 | 1SG | 3188 |
| ATOM | 3188 | CZ | ARG | 389 | 5.807 | 33.695 | 35.494 | 1.00 | 0.74 | 1SG | 3189 |
| ATOM | 3189 | NH1 | ARG | 389 | 5.189 | 34.819 | 35.027 | 1.00 | 0.74 | 1SG | 3190 |
| ATOM | 3190 | NH2 | ARG | 389 | 7.072 | 33.392 | 35.083 | 1.00 | 0.74 | 1SG | 3191 |
| ATOM | 3191 | C | ARG | 389 | 1.512 | 29.939 | 38.740 | 1.00 | 0.74 | 1SG | 3192 |
| ATOM | 3192 | O | ARG | 389 | 1.171 | 30.281 | 39.871 | 1.00 | 0.74 | 1SG | 3193 |
| ATOM | 3193 | N | ASP | 390 | 0.645 | 29.730 | 37.732 | 1.00 | 0.77 | 1SG | 3194 |
| ATOM | 3194 | CA | ASP | 390 | −0.767 | 29.840 | 37.910 | 1.00 | 0.77 | 1SG | 3195 |
| ATOM | 3195 | CB | ASP | 390 | −1.268 | 31.296 | 37.876 | 1.00 | 0.77 | 1SG | 3196 |
| ATOM | 3196 | CG | ASP | 390 | −0.876 | 31.976 | 39.181 | 1.00 | 0.77 | 1SG | 3197 |
| ATOM | 3197 | OD1 | ASP | 390 | −1.094 | 31.362 | 40.259 | 1.00 | 0.77 | 1SG | 3198 |
| ATOM | 3198 | OD2 | ASP | 390 | −0.350 | 33.120 | 39.116 | 1.00 | 0.77 | 1SG | 3199 |
| ATOM | 3199 | C | ASP | 390 | −1.425 | 29.136 | 36.763 | 1.00 | 0.77 | 1SG | 3200 |
| ATOM | 3200 | O | ASP | 390 | −2.593 | 28.768 | 36.861 | 1.00 | 0.77 | 1SG | 3201 |

TABLE 8
Core 3 model

| REMARK Produced by MODELLER: 21 Feb. 2000 18:26:46 | | | | | | | | | | | 1 |
|---|---|---|---|---|---|---|---|---|---|---|---|
| REMARK MODELLER OBJECTIVE FUNCTION: 3243.0485 | | | | | | | | | | | |
| ATOM | 1 | N | MET | 1 | −33.616 | −14.573 | 18.384 | 1.00 | 1.22 | 1SG | 2 |
| ATOM | 2 | CA | MET | 1 | −33.694 | −14.010 | 19.750 | 1.00 | 1.22 | 1SG | 3 |
| ATOM | 3 | CB | MET | 1 | −32.451 | −14.410 | 20.561 | 1.00 | 1.22 | 1SG | 4 |
| ATOM | 4 | CG | MET | 1 | −31.152 | −13.810 | 20.020 | 1.00 | 1.22 | 1SG | 5 |
| ATOM | 5 | SD | MET | 1 | −29.663 | −14.263 | 20.961 | 1.00 | 1.22 | 1SG | 6 |
| ATOM | 6 | CE | MET | 1 | −30.093 | −13.274 | 22.422 | 1.00 | 1.22 | 1SG | 7 |
| ATOM | 7 | C | MET | 1 | −33.772 | −12.521 | 19.694 | 1.00 | 1.22 | 1SG | 8 |
| ATOM | 8 | O | MET | 1 | −34.299 | −11.953 | 18.739 | 1.00 | 1.22 | 1SG | 9 |
| ATOM | 9 | N | ALA | 2 | −33.253 | −11.850 | 20.735 | 1.00 | 0.94 | 1SG | 10 |
| ATOM | 10 | CA | ALA | 2 | −33.294 | −10.420 | 20.745 | 1.00 | 0.94 | 1SG | 11 |
| ATOM | 11 | CB | ALA | 2 | −33.058 | −9.804 | 22.134 | 1.00 | 0.94 | 1SG | 12 |
| ATOM | 12 | C | ALA | 2 | −32.204 | −9.944 | 19.847 | 1.00 | 0.94 | 1SG | 13 |
| ATOM | 13 | O | ALA | 2 | −31.357 | −10.724 | 19.414 | 1.00 | 0.94 | 1SG | 14 |
| ATOM | 14 | N | PHE | 3 | −32.214 | −8.638 | 19.526 | 1.00 | 0.82 | 1SG | 15 |
| ATOM | 15 | CA | PHE | 3 | −31.193 | −8.108 | 18.677 | 1.00 | 0.82 | 1SG | 16 |
| ATOM | 16 | CB | PHE | 3 | −31.613 | −6.827 | 17.935 | 1.00 | 0.82 | 1SG | 17 |
| ATOM | 17 | CG | PHE | 3 | −31.903 | −5.791 | 18.965 | 1.00 | 0.82 | 1SG | 18 |
| ATOM | 18 | CG1 | PHE | 3 | −33.118 | −5.767 | 19.610 | 1.00 | 0.82 | 1SG | 19 |
| ATOM | 19 | CD2 | PHE | 3 | −30.960 | −4.841 | 19.283 | 1.00 | 0.82 | 1SG | 20 |
| ATOM | 20 | CE1 | PHE | 3 | −33.388 | −4.810 | 20.560 | 1.00 | 0.82 | 1SG | 21 |
| ATOM | 21 | CE2 | PHE | 3 | −31.224 | −3.882 | 20.232 | 1.00 | 0.82 | 1SG | 22 |
| ATOM | 22 | CZ | PHE | 3 | −32.440 | −3.866 | 20.873 | 1.00 | 0.82 | 1SG | 23 |
| ATOM | 23 | C | PHE | 3 | −30.034 | −7.770 | 19.550 | 1.00 | 0.82 | 1SG | 24 |
| ATOM | 24 | O | PHE | 3 | −30.178 | −7.534 | 20.748 | 1.00 | 0.82 | 1SG | 25 |
| ATOM | 25 | N | PRO | 4 | −28.873 | −7.781 | 18.963 | 1.00 | 0.79 | 1SG | 26 |
| ATOM | 26 | CA | PRO | 4 | −27.694 | −7.451 | 19.710 | 1.00 | 0.79 | 1SG | 27 |
| ATOM | 27 | CD | PRO | 4 | −28.593 | −8.720 | 17.891 | 1.00 | 0.79 | 1SG | 28 |
| ATOM | 28 | CB | PRO | 4 | −26.530 | −8.160 | 19.012 | 1.00 | 0.79 | 1SG | 29 |
| ATOM | 29 | CG | PRO | 4 | −27.088 | −8.565 | 17.637 | 1.00 | 0.79 | 1SG | 30 |
| ATOM | 30 | C | PRO | 4 | −27.547 | −5.968 | 19.750 | 1.00 | 0.79 | 1SG | 31 |
| ATOM | 31 | O | PRO | 4 | −28.113 | −5.292 | 18.892 | 1.00 | 0.79 | 1SG | 32 |
| ATOM | 32 | N | LYS | 5 | −26.807 | −5.439 | 20.741 | 1.00 | 0.94 | 1SG | 33 |
| ATOM | 33 | CA | LYS | 5 | −26.609 | −4.024 | 20.787 | 1.00 | 0.94 | 1SG | 34 |
| ATOM | 34 | CB | LYS | 5 | −25.941 | −3.541 | 22.086 | 1.00 | 0.94 | 1SG | 35 |
| ATOM | 35 | CG | LYS | 5 | −25.727 | −2.028 | 22.152 | 1.00 | 0.94 | 1SG | 36 |
| ATOM | 36 | CD | LYS | 5 | −27.022 | −1.217 | 22.250 | 1.00 | 0.94 | 1SG | 37 |
| ATOM | 37 | CE | LYS | 5 | −27.800 | −1.147 | 20.935 | 1.00 | 0.94 | 1SG | 38 |
| ATOM | 38 | NZ | LYS | 5 | −29.033 | −0.350 | 21.121 | 1.00 | 0.94 | 1SG | 39 |
| ATOM | 39 | C | LYS | 5 | −25.702 | −3.707 | 19.648 | 1.00 | 0.94 | 1SG | 40 |
| ATOM | 40 | O | LYS | 5 | −24.880 | −4.532 | 19.249 | 1.00 | 0.94 | 1SG | 41 |
| ATOM | 41 | N | MET | 6 | −25.841 | −2.502 | 19.069 | 1.00 | 1.04 | 1SG | 42 |
| ATOM | 42 | CA | MET | 6 | −25.011 | −2.176 | 17.953 | 1.00 | 1.04 | 1SG | 43 |
| ATOM | 43 | CB | MET | 6 | −25.735 | −1.311 | 16.906 | 1.00 | 1.04 | 1SG | 44 |
| ATOM | 44 | CG | MET | 6 | −24.960 | −1.145 | 15.598 | 1.00 | 1.04 | 1SG | 45 |
| ATOM | 45 | SD | MET | 6 | −24.888 | −2.641 | 14.565 | 1.00 | 1.04 | 1SG | 46 |
| ATOM | 46 | CE | MET | 6 | −26.624 | −2.572 | 14.035 | 1.00 | 1.04 | 1SG | 47 |
| ATOM | 47 | C | MET | 6 | −23.845 | −1.400 | 18.476 | 1.00 | 1.04 | 1SG | 48 |
| ATOM | 48 | O | MET | 6 | −23.962 | −0.213 | 18.778 | 1.00 | 1.04 | 1SG | 49 |
| ATOM | 49 | N | ARG | 7 | −22.678 | −2.065 | 18.601 | 1.00 | 0.90 | 1SG | 50 |
| ATOM | 50 | CA | ARG | 7 | −21.505 | −1.406 | 19.100 | 1.00 | 0.90 | 1SG | 51 |
| ATOM | 51 | CB | ARG | 7 | −20.458 | −2.373 | 19.678 | 1.00 | 0.90 | 1SG | 52 |
| ATOM | 52 | CG | ARG | 7 | −20.924 | −3.141 | 20.912 | 1.00 | 0.90 | 1SG | 53 |
| ATOM | 53 | CD | ARG | 7 | −19.932 | −4.216 | 21.355 | 1.00 | 0.90 | 1SG | 54 |
| ATOM | 54 | NE | ARG | 7 | −19.805 | −5.172 | 20.219 | 1.00 | 0.90 | 1SG | 55 |
| ATOM | 55 | CZ | ARG | 7 | −18.957 | −6.236 | 20.304 | 1.00 | 0.90 | 1SG | 56 |
| ATOM | 56 | NH1 | ARG | 7 | −18.224 | −6.434 | 21.438 | 1.00 | 0.90 | 1SG | 57 |
| ATOM | 57 | NH2 | ARG | 7 | −18.843 | −7.103 | 19.255 | 1.00 | 0.90 | 1SG | 58 |
| ATOM | 58 | C | ARG | 7 | −20.851 | −0.765 | 17.932 | 1.00 | 0.90 | 1SG | 59 |
| ATOM | 59 | O | ARG | 7 | −20.261 | −1.443 | 17.090 | 1.00 | 0.90 | 1SG | 60 |
| ATOM | 60 | N | LEU | 8 | −20.932 | 0.570 | 17.833 | 1.00 | 0.75 | 1SG | 61 |
| ATOM | 61 | CA | LEU | 8 | −20.311 | 1.139 | 16.685 | 1.00 | 0.75 | 1SG | 62 |
| ATOM | 62 | CB | LEU | 8 | −21.309 | 1.870 | 15.770 | 1.00 | 0.75 | 1SG | 63 |
| ATOM | 63 | CG | LEU | 8 | −20.767 | 2.155 | 14.361 | 1.00 | 0.75 | 1SG | 64 |
| ATOM | 64 | CD2 | LEU | 8 | −20.514 | 0.836 | 13.620 | 1.00 | 0.75 | 1SG | 65 |
| ATOM | 65 | CD1 | LEU | 8 | −19.539 | 3.074 | 14.386 | 1.00 | 0.75 | 1SG | 66 |
| ATOM | 66 | C | LEU | 8 | −19.323 | 2.143 | 17.172 | 1.00 | 0.75 | 1SG | 67 |
| ATOM | 67 | O | LEU | 8 | −19.677 | 3.090 | 17.872 | 1.00 | 0.75 | 1SG | 68 |
| ATOM | 68 | N | MET | 9 | −18.039 | 1.936 | 16.827 | 1.00 | 0.69 | 1SG | 69 |
| ATOM | 69 | CA | MET | 9 | −17.028 | 2.899 | 17.145 | 1.00 | 0.69 | 1SG | 70 |
| ATOM | 70 | CB | MET | 9 | −15.850 | 2.352 | 17.975 | 1.00 | 0.69 | 1SG | 71 |
| ATOM | 71 | CG | MET | 9 | −16.238 | 1.944 | 19.399 | 1.00 | 0.69 | 1SG | 72 |
| ATOM | 72 | SD | MET | 9 | −14.836 | 1.560 | 20.495 | 1.00 | 0.69 | 1SG | 73 |
| ATOM | 73 | CE | MET | 9 | −14.403 | −0.012 | 19.696 | 1.00 | 0.69 | 1SG | 74 |
| ATOM | 74 | C | MET | 9 | −16.498 | 3.298 | 15.818 | 1.00 | 0.69 | 1SG | 75 |
| ATOM | 75 | O | MET | 9 | −16.399 | 2.461 | 14.925 | 1.00 | 0.69 | 1SG | 76 |
| ATOM | 76 | N | TYR | 10 | −16.164 | 4.584 | 15.621 | 1.00 | 0.75 | 1SG | 77 |
| ATOM | 77 | CA | TYR | 10 | −15.740 | 4.913 | 14.298 | 1.00 | 0.75 | 1SG | 78 |
| ATOM | 78 | CB | TYR | 10 | −16.688 | 5.894 | 13.590 | 1.00 | 0.75 | 1SG | 79 |
| ATOM | 79 | CG | TYR | 10 | −16.519 | 7.249 | 14.200 | 1.00 | 0.75 | 1SG | 80 |
| ATOM | 80 | CD1 | TYR | 10 | −16.669 | 7.465 | 15.552 | 1.00 | 0.75 | 1SG | 81 |
| ATOM | 81 | CD2 | TYR | 10 | −16.230 | 8.330 | 13.396 | 1.00 | 0.75 | 1SG | 82 |
| ATOM | 82 | CE1 | TYR | 10 | −16.531 | 8.733 | 16.064 | 1.00 | 0.75 | 1SG | 83 |
| ATOM | 83 | CE2 | TYR | 10 | −16.086 | 9.600 | 13.900 | 1.00 | 0.75 | 1SG | 84 |
| ATOM | 84 | CZ | TYR | 10 | −16.234 | 9.791 | 15.250 | 1.00 | 0.75 | 1SG | 85 |
| ATOM | 85 | OH | TYR | 10 | −16.102 | 11.071 | 15.819 | 1.00 | 0.75 | 1SG | 86 |
| ATOM | 86 | C | TYR | 10 | −14.415 | 5.577 | 14.363 | 1.00 | 0.75 | 1SG | 87 |
| ATOM | 87 | O | TYR | 10 | −14.140 | 6.388 | 15.247 | 1.00 | 0.75 | 1SG | 88 |
| ATOM | 88 | N | ILE | 11 | −13.522 | 5.183 | 13.442 | 1.00 | 0.67 | 1SG | 89 |
| ATOM | 89 | CA | ILE | 11 | −12.321 | 5.937 | 13.333 | 1.00 | 0.67 | 1SG | 90 |
| ATOM | 90 | CB | ILE | 11 | −11.099 | 5.084 | 13.094 | 1.00 | 0.67 | 1SG | 91 |
| ATOM | 91 | CG2 | ILE | 11 | −9.870 | 6.008 | 13.039 | 1.00 | 0.67 | 1SG | 92 |
| ATOM | 92 | CG1 | ILE | 11 | −10.972 | 4.004 | 14.185 | 1.00 | 0.67 | 1SG | 93 |
| ATOM | 93 | CD1 | ILE | 11 | −12.006 | 2.884 | 14.070 | 1.00 | 0.67 | 1SG | 94 |
| ATOM | 94 | C | ILE | 11 | −12.593 | 6.735 | 12.102 | 1.00 | 0.67 | 1SG | 95 |
| ATOM | 95 | O | ILE | 11 | −11.715 | 6.996 | 11.283 | 1.00 | 0.67 | 1SG | 96 |
| ATOM | 96 | N | CYS | 12 | −13.864 | 7.153 | 11.949 | 1.00 | 0.69 | 1SG | 97 |
| ATOM | 97 | CA | CYS | 12 | −14.229 | 7.892 | 10.782 | 1.00 | 0.69 | 1SG | 98 |
| ATOM | 98 | CB | CYS | 12 | −15.739 | 8.003 | 10.522 | 1.00 | 0.69 | 1SG | 99 |
| ATOM | 99 | SG | CYS | 12 | −16.080 | 8.977 | 9.032 | 1.00 | 0.69 | 1SG | 100 |
| ATOM | 100 | C | CYS | 12 | −13.677 | 9.257 | 10.932 | 1.00 | 0.69 | 1SG | 101 |
| ATOM | 101 | O | CYS | 12 | −13.439 | 9.933 | 9.932 | 1.00 | 0.69 | 1SG | 102 |
| ATOM | 102 | N | LEU | 13 | −13.515 | 9.718 | 12.189 | 1.00 | 0.74 | 1SG | 103 |
| ATOM | 103 | CA | LEU | 13 | −12.873 | 10.986 | 12.328 | 1.00 | 0.74 | 1SG | 104 |
| ATOM | 104 | CB | LEU | 13 | −13.071 | 11.695 | 13.678 | 1.00 | 0.74 | 1SG | 105 |
| ATOM | 105 | CG | LEU | 13 | −14.484 | 12.261 | 13.879 | 1.00 | 0.74 | 1SG | 106 |
| ATOM | 106 | CD2 | LEU | 13 | −15.020 | 12.919 | 12.603 | 1.00 | 0.74 | 1SG | 107 |
| ATOM | 107 | CD1 | LEU | 13 | −14.518 | 13.217 | 15.076 | 1.00 | 0.74 | 1SG | 108 |
| ATOM | 108 | C | LEU | 13 | −11.425 | 10.710 | 12.169 | 1.00 | 0.74 | 1SG | 109 |
| ATOM | 109 | O | LEU | 13 | −10.637 | 10.785 | 13.112 | 1.00 | 0.74 | 1SG | 110 |
| ATOM | 110 | N | LEU | 14 | −11.077 | 10.375 | 10.919 | 1.00 | 0.54 | 1SG | 111 |
| ATOM | 111 | CA | LEU | 14 | −9.778 | 10.057 | 10.442 | 1.00 | 0.54 | 1SG | 112 |
| ATOM | 112 | CB | LEU | 14 | −9.729 | 8.630 | 9.874 | 1.00 | 0.54 | 1SG | 113 |
| ATOM | 113 | CG | LEU | 14 | −8.368 | 8.187 | 9.327 | 1.00 | 0.54 | 1SG | 114 |
| ATOM | 114 | CD2 | LEU | 14 | −8.525 | 6.881 | 8.538 | 1.00 | 0.54 | 1SG | 115 |
| ATOM | 115 | CD1 | LEU | 14 | −7.309 | 8.095 | 10.440 | 1.00 | 0.54 | 1SG | 116 |
| ATOM | 116 | C | LEU | 14 | −9.612 | 11.004 | 9.309 | 1.00 | 0.54 | 1SG | 117 |
| ATOM | 117 | O | LEU | 14 | −10.531 | 11.178 | 8.507 | 1.00 | 0.54 | 1SG | 118 |
| ATOM | 118 | N | VAL | 15 | −8.452 | 11.671 | 9.230 | 1.00 | 0.53 | 1SG | 119 |
| ATOM | 119 | CA | VAL | 15 | −8.315 | 12.596 | 8.155 | 1.00 | 0.53 | 1SG | 120 |
| ATOM | 120 | CB | VAL | 15 | −7.569 | 13.853 | 8.523 | 1.00 | 0.53 | 1SG | 121 |
| ATOM | 121 | CG1 | VAL | 15 | −6.181 | 13.498 | 9.090 | 1.00 | 0.53 | 1SG | 122 |
| ATOM | 122 | CG2 | VAL | 15 | −7.516 | 14.757 | 7.280 | 1.00 | 0.53 | 1SG | 123 |
| ATOM | 123 | C | VAL | 15 | −7.595 | 11.913 | 7.040 | 1.00 | 0.53 | 1SG | 124 |
| ATOM | 124 | O | VAL | 15 | −6.418 | 11.574 | 7.161 | 1.00 | 0.53 | 1SG | 125 |
| ATOM | 125 | N | LEU | 16 | −8.338 | 11.647 | 5.944 | 1.00 | 0.59 | 1SG | 126 |
| ATOM | 126 | CA | LEU | 16 | −7.841 | 11.072 | 4.724 | 1.00 | 0.59 | 1SG | 127 |
| ATOM | 127 | CB | LEU | 16 | −6.337 | 11.254 | 4.452 | 1.00 | 0.59 | 1SG | 128 |
| ATOM | 128 | CG | LEU | 16 | −5.960 | 12.733 | 4.245 | 1.00 | 0.59 | 1SG | 129 |
| ATOM | 129 | CD2 | LEU | 16 | −6.938 | 13.430 | 3.284 | 1.00 | 0.59 | 1SG | 130 |
| ATOM | 130 | CD1 | LEU | 16 | −4.491 | 12.881 | 3.820 | 1.00 | 0.59 | 1SG | 131 |
| ATOM | 131 | C | LEU | 16 | −8.190 | 9.619 | 4.683 | 1.00 | 0.59 | 1SG | 132 |
| ATOM | 132 | O | LEU | 16 | −9.220 | 9.215 | 5.222 | 1.00 | 0.59 | 1SG | 133 |
| ATOM | 133 | N | GLY | 17 | −7.333 | 8.801 | 4.023 | 1.00 | 0.54 | 1SG | 134 |
| ATOM | 134 | CA | GLY | 17 | −7.579 | 7.388 | 3.928 | 1.00 | 0.54 | 1SG | 135 |
| ATOM | 135 | C | GLY | 17 | −6.444 | 6.725 | 3.202 | 1.00 | 0.54 | 1SG | 136 |
| ATOM | 136 | O | GLY | 17 | −6.637 | 5.698 | 2.553 | 1.00 | 0.54 | 1SG | 137 |
| ATOM | 137 | N | ALA | 18 | −5.216 | 7.270 | 3.306 | 1.00 | 0.55 | 1SG | 138 |
| ATOM | 138 | CA | ALA | 18 | −4.134 | 6.681 | 2.571 | 1.00 | 0.55 | 1SG | 139 |
| ATOM | 139 | CB | ALA | 18 | −2.797 | 7.400 | 2.825 | 1.00 | 0.55 | 1SG | 140 |
| ATOM | 140 | C | ALA | 18 | −3.960 | 5.248 | 2.974 | 1.00 | 0.55 | 1SG | 141 |
| ATOM | 141 | O | ALA | 18 | −4.121 | 4.341 | 2.165 | 1.00 | 0.55 | 1SG | 142 |
| ATOM | 142 | N | LEU | 19 | −3.604 | 4.984 | 4.237 | 1.00 | 0.70 | 1SG | 143 |
| ATOM | 143 | CA | LEU | 19 | −3.436 | 3.618 | 4.630 | 1.00 | 0.70 | 1SG | 144 |
| ATOM | 144 | CB | LEU | 19 | −2.586 | 3.483 | 5.916 | 1.00 | 0.70 | 1SG | 145 |
| ATOM | 145 | CG | LEU | 19 | −2.087 | 2.060 | 6.259 | 1.00 | 0.70 | 1SG | 146 |
| ATOM | 146 | CD2 | LEU | 19 | −3.202 | 1.007 | 6.322 | 1.00 | 0.70 | 1SG | 147 |
| ATOM | 147 | CG1 | LEU | 19 | −1.264 | 2.093 | 7.551 | 1.00 | 0.70 | 1SG | 148 |
| ATOM | 148 | C | LEU | 19 | −4.782 | 3.006 | 4.870 | 1.00 | 0.70 | 1SG | 149 |
| ATOM | 149 | O | LEU | 19 | −5.119 | 1.978 | 4.285 | 1.00 | 0.70 | 1SG | 150 |
| ATOM | 150 | N | CYS | 20 | −5.578 | 3.687 | 5.723 | 1.00 | 1.03 | 1SG | 151 |
| ATOM | 151 | CA | CYS | 20 | −6.898 | 3.336 | 6.155 | 1.00 | 1.03 | 1SG | 152 |
| ATOM | 152 | CB | CYS | 20 | −8.017 | 3.968 | 5.311 | 1.00 | 1.03 | 1SG | 153 |
| ATOM | 153 | SG | CYS | 20 | −9.663 | 3.478 | 5.901 | 1.00 | 1.03 | 1SG | 154 |
| ATOM | 154 | C | CYS | 20 | −7.093 | 1.872 | 6.169 | 1.00 | 1.03 | 1SG | 155 |
| ATOM | 155 | O | CYS | 20 | −7.693 | 1.305 | 5.258 | 1.00 | 1.03 | 1SG | 156 |
| ATOM | 156 | N | LEU | 21 | −6.581 | 1.211 | 7.223 | 1.00 | 1.27 | 1SG | 157 |
| ATOM | 157 | CA | LEU | 21 | −6.812 | −0.195 | 7.338 | 1.00 | 1.27 | 1SG | 158 |
| ATOM | 158 | CB | LEU | 21 | −5.565 | −1.058 | 7.101 | 1.00 | 1.27 | 1SG | 159 |
| ATOM | 159 | CG | LEU | 21 | −5.842 | −2.566 | 7.230 | 1.00 | 1.27 | 1SG | 160 |
| ATOM | 160 | CD2 | LEU | 21 | −4.543 | −3.384 | 7.196 | 1.00 | 1.27 | 1SG | 161 |
| ATOM | 161 | CG1 | LEU | 21 | −6.848 | −3.025 | 6.167 | 1.00 | 1.27 | 1SG | 162 |
| ATOM | 162 | C | LEU | 21 | −7.275 | −0.439 | 8.741 | 1.00 | 1.27 | 1SG | 163 |
| ATOM | 163 | O | LEU | 21 | −7.138 | 0.426 | 9.601 | 1.00 | 1.27 | 1SG | 164 |
| ATOM | 164 | N | TYR | 22 | −7.852 | −1.627 | 9.009 | 1.00 | 1.13 | 1SG | 165 |
| ATOM | 165 | CA | TYR | 22 | −8.339 | −1.950 | 10.323 | 1.00 | 1.13 | 1SG | 166 |
| ATOM | 166 | CB | TYR | 22 | −9.749 | −1.401 | 10.619 | 1.00 | 1.13 | 1SG | 167 |
| ATOM | 167 | CG | TYR | 22 | −9.715 | 0.077 | 10.466 | 1.00 | 1.13 | 1SG | 168 |
| ATOM | 168 | CD1 | TYR | 22 | −9.357 | 0.875 | 11.526 | 1.00 | 1.13 | 1SG | 169 |
| ATOM | 169 | CD2 | TYR | 22 | −10.022 | 0.656 | 9.256 | 1.00 | 1.13 | 1SG | 170 |
| ATOM | 170 | CE1 | TYR | 22 | −9.313 | 2.240 | 11.386 | 1.00 | 1.13 | 1SG | 171 |
| ATOM | 171 | CE2 | TYR | 22 | −9.982 | 2.020 | 9.109 | 1.00 | 1.13 | 1SG | 172 |
| ATOM | 172 | CZ | TYR | 22 | −9.630 | 2.809 | 10.176 | 1.00 | 1.13 | 1SG | 173 |
| ATOM | 173 | OH | TYR | 22 | −9.588 | 4.209 | 10.020 | 1.00 | 1.13 | 1SG | 174 |
| ATOM | 174 | C | TYR | 22 | −8.502 | −3.431 | 10.328 | 1.00 | 1.13 | 1SG | 175 |
| ATOM | 175 | O | TYR | 22 | −7.800 | −4.143 | 9.611 | 1.00 | 1.13 | 1SG | 176 |
| ATOM | 176 | N | PHE | 23 | −9.413 | −3.939 | 11.184 | 1.00 | 0.72 | 1SG | 177 |
| ATOM | 177 | CA | PHE | 23 | −9.697 | −5.342 | 11.171 | 1.00 | 0.72 | 1SG | 178 |
| ATOM | 178 | CB | PHE | 23 | −10.629 | −5.787 | 12.314 | 1.00 | 0.72 | 1SG | 179 |
| ATOM | 179 | CG | PHE | 23 | −10.880 | −7.249 | 12.144 | 1.00 | 0.72 | 1SG | 180 |
| ATOM | 180 | CD1 | PHE | 23 | −11.936 | −7.689 | 11.379 | 1.00 | 0.72 | 1SG | 181 |
| ATOM | 181 | CD2 | PHE | 23 | −10.061 | −8.181 | 12.737 | 1.00 | 0.72 | 1SG | 182 |
| ATOM | 182 | CE1 | PHE | 23 | −12.172 | −9.034 | 11.212 | 1.00 | 0.72 | 1SG | 183 |
| ATOM | 183 | CE2 | PHE | 23 | −10.291 | −9.527 | 12.581 | 1.00 | 0.72 | 1SG | 184 |
| ATOM | 184 | CZ | PHE | 23 | −11.354 | −9.953 | 11.814 | 1.00 | 0.72 | 1SG | 185 |
| ATOM | 185 | C | PHE | 23 | −10.378 | −5.625 | 9.878 | 1.00 | 0.72 | 1SG | 186 |
| ATOM | 186 | O | PHE | 23 | −10.034 | −6.563 | 9.163 | 1.00 | 0.72 | 1SG | 187 |
| ATOM | 187 | N | SER | 24 | −11.374 | −4.786 | 9.533 | 1.00 | 0.53 | 1SG | 188 |
| ATOM | 188 | CA | SER | 24 | −12.062 | −4.947 | 8.290 | 1.00 | 0.53 | 1SG | 189 |
| ATOM | 189 | CB | SER | 24 | −13.277 | −5.886 | 8.373 | 1.00 | 0.53 | 1SG | 190 |
| ATOM | 190 | OG | SER | 24 | −13.921 | −5.953 | 7.112 | 1.00 | 0.53 | 1SG | 191 |
| ATOM | 191 | C | SER | 24 | −12.563 | −3.600 | 7.900 | 1.00 | 0.53 | 1SG | 192 |
| ATOM | 192 | O | SER | 24 | −13.090 | −2.859 | 8.725 | 1.00 | 0.53 | 1SG | 193 |
| ATOM | 193 | N | MET | 25 | −12.410 | −3.253 | 6.607 | 1.00 | 0.72 | 1SG | 194 |
| ATOM | 194 | CA | MET | 25 | −12.840 | −1.964 | 6.179 | 1.00 | 0.72 | 1SG | 195 |
| ATOM | 195 | CB | MET | 25 | −12.628 | −1.750 | 4.670 | 1.00 | 0.72 | 1SG | 196 |
| ATOM | 196 | CG | MET | 25 | −11.163 | −1.776 | 4.227 | 1.00 | 0.72 | 1SG | 197 |
| ATOM | 197 | SD | MET | 25 | −10.157 | −0.395 | 4.846 | 1.00 | 0.72 | 1SG | 198 |
| ATOM | 198 | CE | MET | 25 | −10.843 | 0.845 | 3.709 | 1.00 | 0.72 | 1SG | 199 |
| ATOM | 199 | C | MET | 25 | −14.303 | −1.894 | 6.452 | 1.00 | 0.72 | 1SG | 200 |
| ATOM | 200 | O | MET | 25 | −14.792 | −0.940 | 7.057 | 1.00 | 0.72 | 1SG | 201 |
| ATOM | 201 | N | TYR | 26 | −15.036 | −2.941 | 6.033 | 1.00 | 0.81 | 1SG | 202 |
| ATOM | 202 | CA | TYR | 26 | −16.440 | −2.968 | 6.285 | 1.00 | 0.81 | 1SG | 203 |
| ATOM | 203 | CB | TYR | 26 | −17.223 | −3.839 | 5.289 | 1.00 | 0.81 | 1SG | 204 |
| ATOM | 204 | CG | TYR | 26 | −18.621 | −3.960 | 5.790 | 1.00 | 0.81 | 1SG | 205 |
| ATOM | 205 | CD1 | TYR | 26 | −19.554 | −2.977 | 5.553 | 1.00 | 0.81 | 1SG | 206 |
| ATOM | 206 | CD2 | TYR | 26 | −18.996 | −5.075 | 6.503 | 1.00 | 0.81 | 1SG | 207 |
| ATOM | 207 | CE1 | TYR | 26 | −20.841 | −3.110 | 6.022 | 1.00 | 0.81 | 1SG | 208 |
| ATOM | 208 | CE2 | TYR | 26 | −20.278 | −5.213 | 6.973 | 1.00 | 0.81 | 1SG | 209 |
| ATOM | 209 | CZ | TYR | 26 | −21.205 | −4.229 | 6.733 | 1.00 | 0.81 | 1SG | 210 |
| ATOM | 210 | OH | TYR | 26 | −22.526 | −4.365 | 7.212 | 1.00 | 0.81 | 1SG | 211 |
| ATOM | 211 | C | TYR | 26 | −16.623 | −3.551 | 7.641 | 1.00 | 0.81 | 1SG | 212 |
| ATOM | 212 | O | TYR | 26 | −16.612 | −4.768 | 7.817 | 1.00 | 0.81 | 1SG | 213 |
| ATOM | 213 | N | SER | 27 | −16.775 | −2.678 | 8.652 | 1.00 | 0.66 | 1SG | 214 |
| ATOM | 214 | CA | SER | 27 | −16.991 | −3.158 | 9.982 | 1.00 | 0.66 | 1SG | 215 |
| ATOM | 215 | CB | SER | 27 | −15.704 | −3.517 | 10.740 | 1.00 | 0.66 | 1SG | 216 |
| ATOM | 216 | OG | SER | 27 | −15.039 | −4.598 | 10.103 | 1.00 | 0.66 | 1SG | 217 |
| ATOM | 217 | C | SER | 27 | −17.618 | −2.049 | 10.744 | 1.00 | 0.66 | 1SG | 218 |
| ATOM | 218 | O | SER | 27 | −17.607 | −0.896 | 10.315 | 1.00 | 0.66 | 1SG | 219 |
| ATOM | 219 | N | LEU | 28 | −18.211 | −2.388 | 11.900 | 1.00 | 0.42 | 1SG | 220 |
| ATOM | 220 | CA | LEU | 28 | −18.796 | −1.374 | 12.713 | 1.00 | 0.42 | 1SG | 221 |
| ATOM | 221 | CB | LEU | 28 | −19.508 | −1.938 | 13.949 | 1.00 | 0.42 | 1SG | 222 |
| ATOM | 222 | CG | LEU | 28 | −20.750 | −2.779 | 13.608 | 1.00 | 0.42 | 1SG | 223 |
| ATOM | 223 | CD2 | LEU | 28 | −21.710 | −2.005 | 12.696 | 1.00 | 0.42 | 1SG | 224 |
| ATOM | 224 | CD1 | LEU | 28 | −21.424 | −3.309 | 14.880 | 1.00 | 0.42 | 1SG | 225 |
| ATOM | 225 | C | LEU | 28 | −17.686 | −0.502 | 13.190 | 1.00 | 0.42 | 1SG | 226 |
| ATOM | 226 | O | LEU | 28 | −17.779 | 0.725 | 13.163 | 1.00 | 0.42 | 1SG | 227 |
| ATOM | 227 | N | ASN | 29 | −16.580 | −1.124 | 13.629 | 1.00 | 0.36 | 1SG | 228 |
| ATOM | 228 | CA | ASN | 29 | −15.497 | −0.348 | 14.147 | 1.00 | 0.36 | 1SG | 229 |
| ATOM | 229 | CB | ASN | 29 | −14.387 | −1.210 | 14.777 | 1.00 | 0.36 | 1SG | 230 |
| ATOM | 230 | CG | ASN | 29 | −14.939 | −1.841 | 16.049 | 1.00 | 0.36 | 1SG | 231 |
| ATOM | 231 | OD1 | ASN | 29 | −14.786 | −1.296 | 17.141 | 1.00 | 0.36 | 1SG | 232 |
| ATOM | 232 | ND2 | ASN | 29 | −15.609 | −3.016 | 15.908 | 1.00 | 0.36 | 1SG | 233 |
| ATOM | 233 | C | ASN | 29 | −14.928 | 0.482 | 13.039 | 1.00 | 0.36 | 1SG | 234 |
| ATOM | 234 | O | ASN | 29 | −14.699 | 1.674 | 13.230 | 1.00 | 0.36 | 1SG | 235 |
| ATOM | 235 | N | PRO | 30 | −14.659 | −0.042 | 11.879 | 1.00 | 0.63 | 1SG | 236 |
| ATOM | 236 | CA | PRO | 30 | −14.154 | 0.906 | 10.930 | 1.00 | 0.63 | 1SG | 237 |
| ATOM | 237 | CD | PRO | 30 | −13.926 | −1.298 | 11.796 | 1.00 | 0.63 | 1SG | 238 |
| ATOM | 238 | CB | PRO | 30 | −13.229 | 0.147 | 9.984 | 1.00 | 0.63 | 1SG | 239 |
| ATOM | 239 | CG | PRO | 30 | −12.755 | −1.035 | 10.838 | 1.00 | 0.63 | 1SG | 240 |
| ATOM | 240 | C | PRO | 30 | −15.200 | 1.718 | 10.234 | 1.00 | 0.63 | 1SG | 241 |
| ATOM | 241 | O | PRO | 30 | −16.206 | 1.170 | 9.785 | 1.00 | 0.63 | 1SG | 242 |
| ATOM | 242 | N | PHE | 31 | −14.948 | 3.036 | 10.135 | 1.00 | 0.86 | 1SG | 243 |
| ATOM | 243 | CA | PHE | 31 | −15.733 | 3.996 | 9.416 | 1.00 | 0.86 | 1SG | 244 |
| ATOM | 244 | CB | PHE | 31 | −16.847 | 4.674 | 10.234 | 1.00 | 0.86 | 1SG | 245 |
| ATOM | 245 | CG | PHE | 31 | −18.035 | 3.775 | 10.213 | 1.00 | 0.86 | 1SG | 246 |
| ATOM | 246 | CD1 | PHE | 31 | −18.841 | 3.761 | 9.095 | 1.00 | 0.86 | 1SG | 247 |
| ATOM | 247 | CD2 | PHE | 31 | −18.358 | 2.970 | 11.282 | 1.00 | 0.86 | 1SG | 248 |
| ATOM | 248 | CE1 | PHE | 31 | −19.949 | 2.953 | 9.032 | 1.00 | 0.86 | 1SG | 249 |
| ATOM | 249 | CE2 | PHE | 31 | −19.469 | 2.164 | 11.213 | 1.00 | 0.86 | 1SG | 250 |
| ATOM | 250 | CZ | PHE | 31 | −20.268 | 2.147 | 10.096 | 1.00 | 0.86 | 1SG | 251 |
| ATOM | 251 | C | PHE | 31 | −14.752 | 5.041 | 9.008 | 1.00 | 0.86 | 1SG | 252 |
| ATOM | 252 | O | PHE | 31 | −13.847 | 5.358 | 9.777 | 1.00 | 0.86 | 1SG | 253 |
| ATOM | 253 | N | LYS | 32 | −14.876 | 5.597 | 7.783 | 1.00 | 0.81 | 1SG | 254 |
| ATOM | 254 | CA | LYS | 32 | −13.866 | 6.536 | 7.374 | 1.00 | 0.81 | 1SG | 255 |
| ATOM | 255 | CB | LYS | 32 | −12.866 | 5.944 | 6.364 | 1.00 | 0.81 | 1SG | 256 |
| ATOM | 256 | CG | LYS | 32 | −13.478 | 5.489 | 5.031 | 1.00 | 0.81 | 1SG | 257 |
| ATOM | 257 | CD | LYS | 32 | −14.471 | 4.324 | 5.148 | 1.00 | 0.81 | 1SG | 258 |
| ATOM | 258 | CE | LYS | 32 | −14.849 | 3.706 | 3.794 | 1.00 | 0.81 | 1SG | 259 |
| ATOM | 259 | NZ | LYS | 32 | −15.798 | 2.583 | 3.974 | 1.00 | 0.81 | 1SG | 260 |
| ATOM | 260 | C | LYS | 32 | −14.488 | 7.750 | 6.739 | 1.00 | 0.81 | 1SG | 261 |
| ATOM | 261 | O | LYS | 32 | −15.474 | 7.644 | 6.010 | 1.00 | 0.81 | 1SG | 262 |
| ATOM | 262 | N | GLU | 33 | −13.910 | 8.944 | 7.042 | 1.00 | 0.67 | 1SG | 263 |
| ATOM | 263 | CA | GLU | 33 | −14.358 | 10.234 | 6.576 | 1.00 | 0.67 | 1SG | 264 |
| ATOM | 264 | CB | GLU | 33 | −14.312 | 11.338 | 7.650 | 1.00 | 0.67 | 1SG | 265 |
| ATOM | 265 | CG | GLU | 33 | −14.723 | 12.713 | 7.113 | 1.00 | 0.67 | 1SG | 266 |
| ATOM | 266 | CD | GLU | 33 | −14.256 | 13.792 | 8.080 | 1.00 | 0.67 | 1SG | 267 |
| ATOM | 267 | OE1 | GLU | 33 | −13.513 | 13.465 | 9.044 | 1.00 | 0.67 | 1SG | 268 |
| ATOM | 268 | OE2 | GLU | 33 | −14.639 | 14.972 | 7.858 | 1.00 | 0.67 | 1SG | 269 |
| ATOM | 269 | C | GLU | 33 | −13.413 | 10.697 | 5.518 | 1.00 | 0.67 | 1SG | 270 |
| ATOM | 270 | O | GLU | 33 | −12.199 | 10.558 | 5.661 | 1.00 | 0.67 | 1SG | 271 |
| ATOM | 271 | N | GLN | 34 | −13.957 | 11.278 | 4.429 | 1.00 | 0.59 | 1SG | 272 |
| ATOM | 272 | CA | GLN | 34 | −13.131 | 11.730 | 3.349 | 1.00 | 0.59 | 1SG | 273 |
| ATOM | 273 | CB | GLN | 34 | −13.608 | 11.270 | 1.959 | 1.00 | 0.59 | 1SG | 274 |
| ATOM | 274 | CG | GLN | 34 | −12.924 | 11.981 | 0.784 | 1.00 | 0.59 | 1SG | 275 |
| ATOM | 275 | CD | GLN | 34 | −11.455 | 11.587 | 0.718 | 1.00 | 0.59 | 1SG | 276 |
| ATOM | 276 | OE1 | GLN | 34 | −10.601 | 12.466 | 0.605 | 1.00 | 0.59 | 1SG | 277 |
| ATOM | 277 | NE2 | GLN | 34 | −11.150 | 10.261 | 0.764 | 1.00 | 0.59 | 1SG | 278 |
| ATOM | 278 | C | GLN | 34 | −13.123 | 13.216 | 3.329 | 1.00 | 0.59 | 1SG | 279 |
| ATOM | 279 | O | GLN | 34 | −14.156 | 13.872 | 3.442 | 1.00 | 0.59 | 1SG | 280 |
| ATOM | 280 | N | SER | 35 | −11.913 | 13.775 | 3.171 | 1.00 | 0.54 | 1SG | 281 |
| ATOM | 281 | CA | SER | 35 | −11.702 | 15.186 | 3.128 | 1.00 | 0.54 | 1SG | 282 |
| ATOM | 282 | CB | SER | 35 | −10.283 | 15.607 | 3.542 | 1.00 | 0.54 | 1SG | 283 |
| ATOM | 283 | OG | SER | 35 | −10.062 | 15.297 | 4.911 | 1.00 | 0.54 | 1SG | 284 |
| ATOM | 284 | C | SER | 35 | −11.904 | 15.623 | 1.723 | 1.00 | 0.54 | 1SG | 285 |
| ATOM | 285 | O | SER | 35 | −12.892 | 15.245 | 1.094 | 1.00 | 0.54 | 1SG | 286 |
| ATOM | 286 | N | PHE | 36 | −10.910 | 16.388 | 1.219 | 1.00 | 0.59 | 1SG | 287 |
| ATOM | 287 | CA | PHE | 36 | −10.877 | 17.118 | −0.019 | 1.00 | 0.59 | 1SG | 288 |
| ATOM | 288 | CB | PHE | 36 | −9.432 | 17.150 | −0.555 | 1.00 | 0.59 | 1SG | 289 |
| ATOM | 289 | CG | PHE | 36 | −8.529 | 17.512 | 0.580 | 1.00 | 0.59 | 1SG | 290 |
| ATOM | 290 | CD1 | PHE | 36 | −8.091 | 16.530 | 1.439 | 1.00 | 0.59 | 1SG | 291 |
| ATOM | 291 | CD2 | PHE | 36 | −8.127 | 18.806 | 0.807 | 1.00 | 0.59 | 1SG | 292 |
| ATOM | 292 | CE1 | PHE | 36 | −7.267 | 16.823 | 2.500 | 1.00 | 0.59 | 1SG | 293 |
| ATOM | 293 | CE2 | PHE | 36 | −7.301 | 19.103 | 1.866 | 1.00 | 0.59 | 1SG | 294 |
| ATOM | 294 | CZ | PHE | 36 | −6.864 | 18.117 | 2.715 | 1.00 | 0.59 | 1SG | 295 |
| ATOM | 295 | C | PHE | 36 | −11.683 | 16.379 | −1.033 | 1.00 | 0.59 | 1SG | 296 |
| ATOM | 296 | O | PHE | 36 | −12.858 | 16.670 | −1.246 | 1.00 | 0.59 | 1SG | 297 |
| ATOM | 297 | N | VAL | 37 | −11.067 | 15.363 | −1.641 | 1.00 | 0.69 | 1SG | 298 |
| ATOM | 298 | CA | VAL | 37 | −11.706 | 14.493 | −2.566 | 1.00 | 0.69 | 1SG | 299 |
| ATOM | 299 | CB | VAL | 37 | −12.691 | 15.145 | −3.502 | 1.00 | 0.69 | 1SG | 300 |
| ATOM | 300 | CG1 | VAL | 37 | −12.020 | 16.354 | −4.180 | 1.00 | 0.69 | 1SG | 301 |
| ATOM | 301 | CG2 | VAL | 37 | −13.183 | 14.066 | −4.487 | 1.00 | 0.69 | 1SG | 302 |
| ATOM | 302 | C | VAL | 37 | −10.580 | 13.925 | −3.339 | 1.00 | 0.69 | 1SG | 303 |
| ATOM | 303 | O | VAL | 37 | −10.449 | 14.142 | −4.544 | 1.00 | 0.69 | 1SG | 304 |
| ATOM | 304 | N | TYR | 38 | −9.698 | 13.187 | −2.646 | 1.00 | 0.62 | 1SG | 305 |
| ATOM | 305 | CA | TYR | 38 | −8.644 | 12.616 | −3.415 | 1.00 | 0.62 | 1SG | 306 |
| ATOM | 306 | CB | TYR | 38 | −7.341 | 12.309 | −2.653 | 1.00 | 0.62 | 1SG | 307 |
| ATOM | 307 | CG | TYR | 38 | −6.643 | 13.616 | −2.485 | 1.00 | 0.62 | 1SG | 308 |
| ATOM | 308 | CG1 | TYR | 38 | −7.097 | 14.522 | −1.559 | 1.00 | 0.62 | 1SG | 309 |
| ATOM | 309 | CD2 | TYR | 38 | −5.537 | 13.939 | −3.245 | 1.00 | 0.62 | 1SG | 310 |
| ATOM | 310 | CE1 | TYR | 38 | −6.467 | 15.732 | −1.398 | 1.00 | 0.62 | 1SG | 311 |
| ATOM | 311 | CE2 | TYR | 38 | −4.900 | 15.150 | −3.087 | 1.00 | 0.62 | 1SG | 312 |
| ATOM | 312 | CZ | TYR | 38 | −5.368 | 16.051 | −2.160 | 1.00 | 0.62 | 1SG | 313 |
| ATOM | 313 | OH | TYR | 38 | −4.727 | 17.298 | −1.984 | 1.00 | 0.62 | 1SG | 314 |
| ATOM | 314 | C | TYR | 38 | −9.201 | 11.385 | −4.025 | 1.00 | 0.62 | 1SG | 315 |
| ATOM | 315 | O | TYR | 38 | −9.903 | 10.597 | −3.393 | 1.00 | 0.62 | 1SG | 316 |
| ATOM | 316 | N | LYS | 39 | −8.893 | 11.259 | −5.325 | 1.00 | 0.63 | 1SG | 317 |
| ATOM | 317 | CA | LYS | 39 | −9.350 | 10.272 | −6.249 | 1.00 | 0.63 | 1SG | 318 |
| ATOM | 318 | CB | LYS | 39 | −8.700 | 10.562 | −7.622 | 1.00 | 0.63 | 1SG | 319 |
| ATOM | 319 | CG | LYS | 39 | −9.407 | 10.037 | −8.871 | 1.00 | 0.63 | 1SG | 320 |
| ATOM | 320 | CD | LYS | 39 | −8.806 | 10.639 | −10.139 | 1.00 | 0.63 | 1SG | 321 |
| ATOM | 321 | CE | LYS | 39 | −8.757 | 12.163 | −10.091 | 1.00 | 0.63 | 1SG | 322 |
| ATOM | 322 | NZ | LYS | 39 | −8.034 | 12.671 | −11.272 | 1.00 | 0.63 | 1SG | 323 |
| ATOM | 323 | C | LYS | 39 | −8.912 | 8.922 | −5.770 | 1.00 | 0.63 | 1SG | 324 |
| ATOM | 324 | O | LYS | 39 | −9.717 | 8.000 | −5.647 | 1.00 | 0.63 | 1SG | 325 |
| ATOM | 325 | N | LYS | 40 | −7.616 | 8.780 | −5.447 | 1.00 | 0.63 | 1SG | 326 |
| ATOM | 326 | CA | LYS | 40 | −7.089 | 7.500 | −5.055 | 1.00 | 0.63 | 1SG | 327 |
| ATOM | 327 | CB | LYS | 40 | −5.593 | 7.534 | −4.701 | 1.00 | 0.63 | 1SG | 328 |
| ATOM | 328 | CG | LYS | 40 | −4.651 | 7.928 | −5.843 | 1.00 | 0.63 | 1SG | 329 |
| ATOM | 329 | CD | LYS | 40 | −4.575 | 6.918 | −6.990 | 1.00 | 0.63 | 1SG | 330 |
| ATOM | 330 | CE | LYS | 40 | −3.514 | 7.286 | −8.031 | 1.00 | 0.63 | 1SG | 331 |
| ATOM | 331 | NZ | LYS | 40 | −2.162 | 6.971 | −7.515 | 1.00 | 0.63 | 1SG | 332 |
| ATOM | 332 | C | LYS | 40 | −7.758 | 7.070 | −3.789 | 1.00 | 0.63 | 1SG | 333 |
| ATOM | 333 | O | LYS | 40 | −8.081 | 5.898 | −3.594 | 1.00 | 0.63 | 1SG | 334 |
| ATOM | 334 | N | ASP | 41 | −7.956 | 8.025 | −2.880 | 1.00 | 0.34 | 1SG | 335 |
| ATOM | 335 | CA | ASP | 41 | −8.498 | 7.644 | −1.608 | 1.00 | 0.34 | 1SG | 336 |
| ATOM | 336 | CB | ASP | 41 | −8.442 | 8.767 | −0.556 | 1.00 | 0.34 | 1SG | 337 |
| ATOM | 337 | CG | ASP | 41 | −6.980 | 8.922 | −0.177 | 1.00 | 0.34 | 1SG | 338 |
| ATOM | 338 | OD1 | ASP | 41 | −6.272 | 7.877 | −0.192 | 1.00 | 0.34 | 1SG | 339 |
| ATOM | 339 | OD2 | ASP | 41 | −6.546 | 10.073 | 0.106 | 1.00 | 0.34 | 1SG | 340 |
| ATOM | 340 | C | ASP | 41 | −9.908 | 7.206 | −1.761 | 1.00 | 0.34 | 1SG | 341 |
| ATOM | 341 | O | ASP | 41 | −10.338 | 6.273 | −1.085 | 1.00 | 0.34 | 1SG | 342 |
| ATOM | 342 | N | GLY | 42 | −10.648 | 7.878 | −2.655 | 1.00 | 0.15 | 1SG | 343 |
| ATOM | 343 | CA | GLY | 42 | −12.030 | 7.573 | −2.828 | 1.00 | 0.15 | 1SG | 344 |
| ATOM | 344 | C | GLY | 42 | −12.149 | 6.144 | −3.259 | 1.00 | 0.15 | 1SG | 345 |
| ATOM | 345 | O | GLY | 42 | −13.044 | 5.423 | −2.819 | 1.00 | 0.15 | 1SG | 346 |
| ATOM | 346 | N | ASN | 43 | −11.225 | 5.699 | −4.125 | 1.00 | 0.27 | 1SG | 347 |
| ATOM | 347 | CA | ASN | 43 | −11.306 | 4.361 | −4.646 | 1.00 | 0.27 | 1SG | 348 |
| ATOM | 348 | CB | ASN | 43 | −10.111 | 4.006 | −5.544 | 1.00 | 0.27 | 1SG | 349 |
| ATOM | 349 | CG | ASN | 43 | −10.032 | 4.922 | −6.757 | 1.00 | 0.27 | 1SG | 350 |
| ATOM | 350 | OD1 | ASN | 43 | −8.971 | 5.494 | −7.010 | 1.00 | 0.27 | 1SG | 351 |
| ATOM | 351 | ND2 | ASN | 43 | −11.148 | 5.031 | −7.530 | 1.00 | 0.27 | 1SG | 352 |
| ATOM | 352 | C | ASN | 43 | −11.209 | 3.374 | −3.530 | 1.00 | 0.27 | 1SG | 353 |
| ATOM | 353 | O | ASN | 43 | −12.063 | 2.504 | −3.358 | 1.00 | 0.27 | 1SG | 354 |
| ATOM | 354 | N | PHE | 44 | −10.149 | 3.506 | −2.721 | 1.00 | 0.34 | 1SG | 355 |
| ATOM | 355 | CA | PHE | 44 | −9.926 | 2.549 | −1.684 | 1.00 | 0.34 | 1SG | 356 |
| ATOM | 356 | CB | PHE | 44 | −8.650 | 2.813 | −0.870 | 1.00 | 0.34 | 1SG | 357 |
| ATOM | 357 | CG | PHE | 44 | −7.497 | 2.175 | −1.565 | 1.00 | 0.34 | 1SG | 358 |
| ATOM | 358 | CD1 | PHE | 44 | −6.837 | 2.808 | −2.593 | 1.00 | 0.34 | 1SG | 359 |
| ATOM | 359 | CD2 | PHE | 44 | −7.069 | 0.931 | −1.161 | 1.00 | 0.34 | 1SG | 360 |
| ATOM | 360 | CE1 | PHE | 44 | −5.771 | 2.199 | −3.213 | 1.00 | 0.34 | 1SG | 361 |
| ATOM | 361 | CE2 | PHE | 44 | −6.005 | 0.316 | −1.772 | 1.00 | 0.34 | 1SG | 362 |
| ATOM | 362 | CZ | PHE | 44 | −5.353 | 0.957 | −2.798 | 1.00 | 0.34 | 1SG | 363 |
| ATOM | 363 | C | PHE | 44 | −11.061 | 2.500 | −0.726 | 1.00 | 0.34 | 1SG | 364 |
| ATOM | 364 | O | PHE | 44 | −11.541 | 1.414 | −0.406 | 1.00 | 0.34 | 1SG | 365 |
| ATOM | 365 | N | LEU | 45 | −11.518 | 3.663 | −0.237 | 1.00 | 0.31 | 1SG | 366 |
| ATOM | 366 | CA | LEU | 45 | −12.552 | 3.700 | 0.754 | 1.00 | 0.31 | 1SG | 367 |
| ATOM | 367 | CB | LEU | 45 | −12.688 | 5.087 | 1.406 | 1.00 | 0.31 | 1SG | 368 |
| ATOM | 368 | CG | LEU | 45 | −11.416 | 5.487 | 2.178 | 1.00 | 0.31 | 1SG | 369 |
| ATOM | 369 | CD2 | LEU | 45 | −10.937 | 4.356 | 3.105 | 1.00 | 0.31 | 1SG | 370 |
| ATOM | 370 | CD1 | LEU | 45 | −11.601 | 6.825 | 2.909 | 1.00 | 0.31 | 1SG | 371 |
| ATOM | 371 | C | LEU | 45 | −13.886 | 3.305 | 0.183 | 1.00 | 0.31 | 1SG | 372 |
| ATOM | 372 | O | LEU | 45 | −14.664 | 2.596 | 0.821 | 1.00 | 0.31 | 1SG | 373 |
| ATOM | 373 | N | LYS | 46 | −14.178 | 3.729 | −1.062 | 1.00 | 0.29 | 1SG | 374 |
| ATOM | 374 | CA | LYS | 46 | −15.484 | 3.527 | −1.626 | 1.00 | 0.29 | 1SG | 375 |
| ATOM | 375 | CB | LYS | 46 | −15.670 | 4.084 | −3.060 | 1.00 | 0.29 | 1SG | 376 |
| ATOM | 376 | CG | LYS | 46 | −15.798 | 5.611 | −3.128 | 1.00 | 0.29 | 1SG | 377 |
| ATOM | 377 | CD | LYS | 46 | −15.899 | 6.178 | −4.547 | 1.00 | 0.29 | 1SG | 378 |
| ATOM | 378 | CE | LYS | 46 | −16.229 | 7.674 | −4.564 | 1.00 | 0.29 | 1SG | 379 |
| ATOM | 379 | NZ | LYS | 46 | −16.585 | 8.115 | −5.932 | 1.00 | 0.29 | 1SG | 380 |
| ATOM | 380 | C | LYS | 46 | −15.815 | 2.078 | −1.647 | 1.00 | 0.29 | 1SG | 381 |
| ATOM | 381 | O | LYS | 46 | −16.963 | 1.705 | −1.422 | 1.00 | 0.29 | 1SG | 382 |
| ATOM | 382 | N | LEU | 47 | −14.831 | 1.204 | −1.904 | 1.00 | 0.35 | 1SG | 383 |
| ATOM | 383 | CA | LEU | 47 | −15.176 | −0.180 | −1.997 | 1.00 | 0.35 | 1SG | 384 |
| ATOM | 384 | CB | LEU | 47 | −13.993 | −1.048 | −2.451 | 1.00 | 0.35 | 1SG | 385 |
| ATOM | 385 | CG | LEU | 47 | −13.534 | −0.657 | −3.869 | 1.00 | 0.35 | 1SG | 386 |
| ATOM | 386 | CD2 | LEU | 47 | −14.737 | −0.495 | −4.815 | 1.00 | 0.35 | 1SG | 387 |
| ATOM | 387 | CD1 | LEU | 47 | −12.461 | −1.617 | −4.405 | 1.00 | 0.35 | 1SG | 388 |
| ATOM | 388 | C | LEU | 47 | −15.710 | −0.682 | −0.684 | 1.00 | 0.35 | 1SG | 389 |
| ATOM | 389 | O | LEU | 47 | −16.683 | −1.435 | −0.668 | 1.00 | 0.35 | 1SG | 390 |
| ATOM | 390 | N | PRO | 48 | −15.120 | −0.313 | 0.419 | 1.00 | 0.59 | 1SG | 391 |
| ATOM | 391 | CA | PRO | 48 | −15.596 | −0.791 | 1.683 | 1.00 | 0.59 | 1SG | 392 |
| ATOM | 392 | CD | PRO | 48 | −13.681 | −0.176 | 0.457 | 1.00 | 0.59 | 1SG | 393 |
| ATOM | 393 | CB | PRO | 48 | −14.517 | −0.443 | 2.704 | 1.00 | 0.59 | 1SG | 394 |
| ATOM | 394 | CG | PRO | 48 | −13.412 | 0.266 | 1.899 | 1.00 | 0.59 | 1SG | 395 |
| ATOM | 395 | C | PRO | 48 | −16.970 | −0.442 | 2.142 | 1.00 | 0.59 | 1SG | 396 |
| ATOM | 396 | O | PRO | 48 | −17.636 | −1.364 | 2.612 | 1.00 | 0.59 | 1SG | 397 |
| ATOM | 397 | N | ASP | 49 | −17.442 | 0.825 | 2.045 | 1.00 | 0.72 | 1SG | 398 |
| ATOM | 398 | CA | ASP | 49 | −18.763 | 1.020 | 2.578 | 1.00 | 0.72 | 1SG | 399 |
| ATOM | 399 | CB | ASP | 49 | −18.898 | 0.608 | 4.055 | 1.00 | 0.72 | 1SG | 400 |
| ATOM | 400 | CG | ASP | 49 | −20.373 | 0.348 | 4.305 | 1.00 | 0.72 | 1SG | 401 |
| ATOM | 401 | OD1 | ASP | 49 | −21.174 | 0.676 | 3.391 | 1.00 | 0.72 | 1SG | 402 |
| ATOM | 402 | OD2 | ASP | 49 | −20.716 | −0.180 | 5.397 | 1.00 | 0.72 | 1SG | 403 |
| ATOM | 403 | C | ASP | 49 | −19.158 | 2.465 | 2.484 | 1.00 | 0.72 | 1SG | 404 |
| ATOM | 404 | O | ASP | 49 | −18.972 | 3.112 | 1.456 | 1.00 | 0.72 | 1SG | 405 |
| ATOM | 405 | N | THR | 50 | −19.729 | 3.003 | 3.586 | 1.00 | 0.52 | 1SG | 406 |
| ATOM | 406 | CA | THR | 50 | −20.266 | 4.337 | 3.607 | 1.00 | 0.52 | 1SG | 407 |
| ATOM | 407 | CB | THR | 50 | −21.195 | 4.592 | 4.756 | 1.00 | 0.52 | 1SG | 408 |
| ATOM | 408 | OG1 | THR | 50 | −22.291 | 3.690 | 4.705 | 1.00 | 0.52 | 1SG | 409 |
| ATOM | 409 | CG2 | THR | 50 | −21.702 | 6.042 | 4.661 | 1.00 | 0.52 | 1SG | 410 |
| ATOM | 410 | C | THR | 50 | −19.164 | 5.337 | 3.688 | 1.00 | 0.52 | 1SG | 411 |
| ATOM | 411 | O | THR | 50 | −18.225 | 5.204 | 4.473 | 1.00 | 0.52 | 1SG | 412 |
| ATOM | 412 | N | ASP | 51 | −19.267 | 6.384 | 2.849 | 1.00 | 0.34 | 1SG | 413 |
| ATOM | 413 | CA | ASP | 51 | −18.245 | 7.381 | 2.831 | 1.00 | 0.34 | 1SG | 414 |
| ATOM | 414 | CB | ASP | 51 | −17.485 | 7.415 | 1.493 | 1.00 | 0.34 | 1SG | 415 |
| ATOM | 415 | CG | ASP | 51 | −16.830 | 6.057 | 1.304 | 1.00 | 0.34 | 1SG | 416 |
| ATOM | 416 | OD1 | ASP | 51 | −16.289 | 5.516 | 2.308 | 1.00 | 0.34 | 1SG | 417 |
| ATOM | 417 | OD2 | ASP | 51 | −16.880 | 5.522 | 0.163 | 1.00 | 0.34 | 1SG | 418 |
| ATOM | 418 | C | ASP | 51 | −18.917 | 8.703 | 2.988 | 1.00 | 0.34 | 1SG | 419 |
| ATOM | 419 | O | ASP | 51 | −19.929 | 8.984 | 2.345 | 1.00 | 0.34 | 1SG | 420 |
| ATOM | 420 | N | CYS | 52 | −18.360 | 9.556 | 3.867 | 1.00 | 0.46 | 1SG | 421 |
| ATOM | 421 | CA | CYS | 52 | −18.920 | 10.858 | 4.044 | 1.00 | 0.46 | 1SG | 422 |
| ATOM | 422 | CB | CYS | 52 | −18.650 | 11.458 | 5.431 | 1.00 | 0.46 | 1SG | 423 |
| ATOM | 423 | SG | CYS | 52 | −19.343 | 13.125 | 5.631 | 1.00 | 0.46 | 1SG | 424 |
| ATOM | 424 | C | CYS | 52 | −18.234 | 11.725 | 3.055 | 1.00 | 0.46 | 1SG | 425 |
| ATOM | 425 | O | CYS | 52 | −17.026 | 11.946 | 3.136 | 1.00 | 0.46 | 1SG | 426 |
| ATOM | 426 | N | ARG | 53 | −18.996 | 12.229 | 2.071 | 1.00 | 0.69 | 1SG | 427 |
| ATOM | 427 | CA | ARG | 53 | −18.360 | 13.053 | 1.098 | 1.00 | 0.69 | 1SG | 428 |
| ATOM | 428 | CB | ARG | 53 | −19.064 | 13.056 | −0.270 | 1.00 | 0.69 | 1SG | 429 |
| ATOM | 429 | CG | ARG | 53 | −19.271 | 11.666 | −0.875 | 1.00 | 0.69 | 1SG | 430 |
| ATOM | 430 | CD | ARG | 53 | −19.540 | 11.687 | −2.383 | 1.00 | 0.69 | 1SG | 431 |
| ATOM | 431 | NE | ARG | 53 | −20.669 | 12.625 | −2.640 | 1.00 | 0.69 | 1SG | 432 |
| ATOM | 432 | CZ | ARG | 53 | −20.967 | 12.991 | −3.920 | 1.00 | 0.69 | 1SG | 433 |
| ATOM | 433 | NH1 | ARG | 53 | −20.285 | 12.438 | −4.966 | 1.00 | 0.69 | 1SG | 434 |
| ATOM | 434 | NH2 | ARG | 53 | −21.940 | 13.919 | −4.159 | 1.00 | 0.69 | 1SG | 435 |
| ATOM | 435 | C | ARG | 53 | −18.453 | 14.439 | 1.616 | 1.00 | 0.69 | 1SG | 436 |
| ATOM | 436 | O | ARG | 53 | −19.490 | 15.089 | 1.499 | 1.00 | 0.69 | 1SG | 437 |
| ATOM | 437 | N | GLN | 54 | −17.368 | 14.934 | 2.229 | 1.00 | 0.77 | 1SG | 438 |
| ATOM | 438 | CA | GLN | 54 | −17.441 | 16.286 | 2.671 | 1.00 | 0.77 | 1SG | 439 |
| ATOM | 439 | CB | GLN | 54 | −16.208 | 16.737 | 3.465 | 1.00 | 0.77 | 1SG | 440 |
| ATOM | 440 | CG | GLN | 54 | −16.224 | 16.275 | 4.915 | 1.00 | 0.77 | 1SG | 441 |
| ATOM | 441 | CD | GLN | 54 | −17.161 | 17.230 | 5.629 | 1.00 | 0.77 | 1SG | 442 |
| ATOM | 442 | OE1 | GLN | 54 | −18.167 | 16.823 | 6.208 | 1.00 | 0.77 | 1SG | 443 |
| ATOM | 443 | NE2 | GLN | 54 | −16.818 | 18.546 | 5.595 | 1.00 | 0.77 | 1SG | 444 |
| ATOM | 444 | C | GLN | 54 | −17.472 | 17.109 | 1.441 | 1.00 | 0.77 | 1SG | 445 |
| ATOM | 445 | O | GLN | 54 | −16.539 | 17.054 | 0.641 | 1.00 | 0.77 | 1SG | 446 |
| ATOM | 446 | N | THR | 55 | −18.560 | 17.876 | 1.240 | 1.00 | 0.77 | 1SG | 447 |
| ATOM | 447 | CA | THR | 55 | −18.509 | 18.742 | 0.111 | 1.00 | 0.77 | 1SG | 448 |
| ATOM | 448 | CB | THR | 55 | −19.755 | 19.557 | −0.115 | 1.00 | 0.77 | 1SG | 449 |
| ATOM | 449 | OG1 | THR | 55 | −19.626 | 20.305 | −1.315 | 1.00 | 0.77 | 1SG | 450 |
| ATOM | 450 | CG2 | THR | 55 | −19.995 | 20.497 | 1.082 | 1.00 | 0.77 | 1SG | 451 |
| ATOM | 451 | C | THR | 55 | −17.384 | 19.643 | 0.460 | 1.00 | 0.77 | 1SG | 452 |
| ATOM | 452 | O | THR | 55 | −17.324 | 20.151 | 1.581 | 1.00 | 0.77 | 1SG | 453 |
| ATOM | 453 | N | PRO | 56 | −16.464 | 19.758 | −0.463 | 1.00 | 0.67 | 1SG | 454 |
| ATOM | 454 | CA | PRO | 56 | −15.255 | 20.506 | −0.271 | 1.00 | 0.67 | 1SG | 455 |
| ATOM | 455 | CD | PRO | 56 | −16.748 | 19.521 | −1.867 | 1.00 | 0.67 | 1SG | 456 |
| ATOM | 456 | CB | PRO | 56 | −14.629 | 20.676 | −1.656 | 1.00 | 0.67 | 1SG | 457 |
| ATOM | 457 | CG | PRO | 56 | −15.378 | 19.666 | −2.548 | 1.00 | 0.67 | 1SG | 458 |
| ATOM | 458 | C | PRO | 56 | −15.597 | 21.816 | 0.334 | 1.00 | 0.67 | 1SG | 459 |
| ATOM | 459 | O | PRO | 56 | −16.059 | 22.702 | −0.386 | 1.00 | 0.67 | 1SG | 460 |
| ATOM | 460 | N | PRO | 57 | −15.403 | 21.902 | 1.628 | 1.00 | 0.40 | 1SG | 461 |
| ATOM | 461 | CA | PRO | 57 | −15.678 | 23.109 | 2.347 | 1.00 | 0.40 | 1SG | 462 |
| ATOM | 462 | CD | PRO | 57 | −14.415 | 21.078 | 2.305 | 1.00 | 0.40 | 1SG | 463 |
| ATOM | 463 | CB | PRO | 57 | −15.134 | 22.883 | 3.755 | 1.00 | 0.40 | 1SG | 464 |
| ATOM | 464 | CG | PRO | 57 | −13.965 | 21.909 | 3.520 | 1.00 | 0.40 | 1SG | 465 |
| ATOM | 465 | C | PRO | 57 | −14.909 | 24.146 | 1.613 | 1.00 | 0.40 | 1SG | 466 |
| ATOM | 466 | O | PRO | 57 | −15.417 | 25.243 | 1.393 | 1.00 | 0.40 | 1SG | 467 |
| ATOM | 467 | N | PHE | 58 | −13.670 | 23.798 | 1.231 | 1.00 | 0.44 | 1SG | 468 |
| ATOM | 468 | CA | PHE | 58 | −12.906 | 24.651 | 0.385 | 1.00 | 0.44 | 1SG | 469 |
| ATOM | 469 | CB | PHE | 58 | −12.332 | 25.909 | 1.060 | 1.00 | 0.44 | 1SG | 470 |
| ATOM | 470 | CG | PHE | 58 | −11.949 | 26.836 | −0.047 | 1.00 | 0.44 | 1SG | 471 |
| ATOM | 471 | CD1 | PHE | 58 | −12.895 | 27.653 | −0.618 | 1.00 | 0.44 | 1SG | 472 |
| ATOM | 472 | CD2 | PHE | 58 | −10.660 | 26.899 | −0.522 | 1.00 | 0.44 | 1SG | 473 |
| ATOM | 473 | CE1 | PHE | 58 | −12.566 | 28.511 | −1.640 | 1.00 | 0.44 | 1SG | 474 |
| ATOM | 474 | CE2 | PHE | 58 | −10.319 | 27.754 | −1.545 | 1.00 | 0.44 | 1SG | 475 |
| ATOM | 475 | CZ | PHE | 58 | −11.275 | 28.563 | −2.106 | 1.00 | 0.44 | 1SG | 476 |
| ATOM | 476 | C | PHE | 58 | −11.762 | 23.820 | −0.080 | 1.00 | 0.44 | 1SG | 477 |
| ATOM | 477 | O | PHE | 58 | −11.407 | 22.825 | 0.551 | 1.00 | 0.44 | 1SG | 478 |
| ATOM | 478 | N | LEU | 59 | −11.177 | 24.197 | −1.226 | 1.00 | 0.77 | 1SG | 479 |
| ATOM | 479 | CA | LEU | 59 | −10.062 | 23.479 | −1.754 | 1.00 | 0.77 | 1SG | 480 |
| ATOM | 480 | CB | LEU | 59 | −9.588 | 24.073 | −3.090 | 1.00 | 0.77 | 1SG | 481 |
| ATOM | 481 | CG | LEU | 59 | −8.387 | 23.347 | −3.715 | 1.00 | 0.77 | 1SG | 482 |
| ATOM | 482 | CD2 | LEU | 59 | −7.773 | 24.185 | −4.846 | 1.00 | 0.77 | 1SG | 483 |
| ATOM | 483 | CD1 | LEU | 59 | −8.753 | 21.920 | −4.153 | 1.00 | 0.77 | 1SG | 484 |
| ATOM | 484 | C | LEU | 59 | −8.945 | 23.621 | −0.773 | 1.00 | 0.77 | 1SG | 485 |
| ATOM | 485 | O | LEU | 59 | −8.269 | 22.651 | −0.436 | 1.00 | 0.77 | 1SG | 486 |
| ATOM | 486 | N | VAL | 60 | −8.736 | 24.853 | −0.274 | 1.00 | 0.78 | 1SG | 487 |
| ATOM | 487 | CA | VAL | 60 | −7.677 | 25.107 | 0.654 | 1.00 | 0.78 | 1SG | 488 |
| ATOM | 488 | CB | VAL | 60 | −6.503 | 25.817 | 0.047 | 1.00 | 0.78 | 1SG | 489 |
| ATOM | 489 | CG1 | VAL | 60 | −5.883 | 24.908 | −1.030 | 1.00 | 0.78 | 1SG | 490 |
| ATOM | 490 | CG2 | VAL | 60 | −6.987 | 27.175 | −0.483 | 1.00 | 0.78 | 1SG | 491 |
| ATOM | 491 | C | VAL | 60 | −8.214 | 25.987 | 1.741 | 1.00 | 0.78 | 1SG | 492 |
| ATOM | 492 | O | VAL | 60 | −9.418 | 26.215 | 1.831 | 1.00 | 0.78 | 1SG | 493 |
| ATOM | 493 | N | LEU | 61 | −7.316 | 26.477 | 2.624 | 1.00 | 0.70 | 1SG | 494 |
| ATOM | 494 | CA | LEU | 61 | −7.723 | 27.320 | 3.715 | 1.00 | 0.70 | 1SG | 495 |
| ATOM | 495 | CB | LEU | 61 | −7.455 | 26.690 | 5.097 | 1.00 | 0.70 | 1SG | 496 |
| ATOM | 496 | CG | LEU | 61 | −8.199 | 25.362 | 5.335 | 1.00 | 0.70 | 1SG | 497 |
| ATOM | 497 | CD2 | LEU | 61 | −8.070 | 24.892 | 6.788 | 1.00 | 0.70 | 1SG | 498 |
| ATOM | 498 | CG1 | LEU | 61 | −7.757 | 24.280 | 4.339 | 1.00 | 0.70 | 1SG | 499 |
| ATOM | 499 | C | LEU | 61 | −6.896 | 28.562 | 3.658 | 1.00 | 0.70 | 1SG | 500 |
| ATOM | 500 | O | LEU | 61 | −5.714 | 28.521 | 3.326 | 1.00 | 0.70 | 1SG | 501 |
| ATOM | 501 | N | LEU | 62 | −7.514 | 29.716 | 3.986 | 1.00 | 0.71 | 1SG | 502 |
| ATOM | 502 | CA | LEU | 62 | −6.768 | 30.938 | 3.976 | 1.00 | 0.71 | 1SG | 503 |
| ATOM | 503 | CB | LEU | 62 | −7.465 | 32.086 | 3.234 | 1.00 | 0.71 | 1SG | 504 |
| ATOM | 504 | CG | LEU | 62 | −6.644 | 33.392 | 3.230 | 1.00 | 0.71 | 1SG | 505 |
| ATOM | 505 | CD2 | LEU | 62 | −7.482 | 34.573 | 2.708 | 1.00 | 0.71 | 1SG | 506 |
| ATOM | 506 | CD1 | LEU | 62 | −5.313 | 33.227 | 2.485 | 1.00 | 0.71 | 1SG | 507 |
| ATOM | 507 | C | LEU | 62 | −6.607 | 31.369 | 5.399 | 1.00 | 0.71 | 1SG | 508 |
| ATOM | 508 | O | LEU | 62 | −7.572 | 31.429 | 6.157 | 1.00 | 0.71 | 1SG | 509 |
| ATOM | 509 | N | VAL | 63 | −5.358 | 31.676 | 5.795 | 1.00 | 0.63 | 1SG | 510 |
| ATOM | 510 | CA | VAL | 63 | −5.112 | 32.105 | 7.138 | 1.00 | 0.63 | 1SG | 511 |
| ATOM | 511 | CB | VAL | 63 | −4.500 | 31.049 | 8.012 | 1.00 | 0.63 | 1SG | 512 |
| ATOM | 512 | CG1 | VAL | 63 | −5.519 | 29.909 | 8.174 | 1.00 | 0.63 | 1SG | 513 |
| ATOM | 513 | CG2 | VAL | 63 | −3.158 | 30.612 | 7.400 | 1.00 | 0.63 | 1SG | 514 |
| ATOM | 514 | C | VAL | 63 | −4.148 | 33.237 | 7.080 | 1.00 | 0.63 | 1SG | 515 |
| ATOM | 515 | O | VAL | 63 | −3.695 | 33.631 | 6.008 | 1.00 | 0.63 | 1SG | 516 |
| ATOM | 516 | N | THR | 64 | −3.833 | 33.805 | 8.258 | 1.00 | 0.55 | 1SG | 517 |
| ATOM | 517 | CA | THR | 64 | −2.912 | 34.895 | 8.321 | 1.00 | 0.55 | 1SG | 518 |
| ATOM | 518 | CB | THR | 64 | −2.812 | 35.511 | 9.686 | 1.00 | 0.55 | 1SG | 519 |
| ATOM | 519 | OG1 | THR | 64 | −1.985 | 36.665 | 9.645 | 1.00 | 0.55 | 1SG | 520 |
| ATOM | 520 | CG2 | THR | 64 | −2.235 | 34.468 | 10.657 | 1.00 | 0.55 | 1SG | 521 |
| ATOM | 521 | C | THR | 64 | −1.565 | 34.370 | 7.951 | 1.00 | 0.55 | 1SG | 522 |
| ATOM | 522 | O | THR | 64 | −1.332 | 33.162 | 7.950 | 1.00 | 0.55 | 1SG | 523 |
| ATOM | 523 | N | SER | 65 | −0.642 | 35.286 | 7.610 | 1.00 | 0.43 | 1SG | 524 |
| ATOM | 524 | CA | SER | 65 | 0.675 | 34.909 | 7.200 | 1.00 | 0.43 | 1SG | 525 |
| ATOM | 525 | CB | SER | 65 | 1.539 | 36.117 | 6.794 | 1.00 | 0.43 | 1SG | 526 |
| ATOM | 526 | OG | SER | 65 | 2.831 | 35.680 | 6.394 | 1.00 | 0.43 | 1SG | 527 |
| ATOM | 527 | C | SER | 65 | 1.354 | 34.238 | 8.348 | 1.00 | 0.43 | 1SG | 528 |
| ATOM | 528 | O | SER | 65 | 2.134 | 33.306 | 8.160 | 1.00 | 0.43 | 1SG | 529 |
| ATOM | 529 | N | SER | 66 | 1.074 | 34.709 | 9.574 | 1.00 | 0.29 | 1SG | 530 |
| ATOM | 530 | CA | SER | 66 | 1.726 | 34.209 | 10.748 | 1.00 | 0.29 | 1SG | 531 |
| ATOM | 531 | CB | SER | 66 | 1.346 | 34.989 | 12.021 | 1.00 | 0.29 | 1SG | 532 |
| ATOM | 532 | OG | SER | 66 | −0.027 | 34.797 | 12.324 | 1.00 | 0.29 | 1SG | 533 |
| ATOM | 533 | C | SER | 66 | 1.392 | 32.767 | 10.987 | 1.00 | 0.29 | 1SG | 534 |
| ATOM | 534 | O | SER | 66 | 2.265 | 31.991 | 11.372 | 1.00 | 0.29 | 1SG | 535 |
| ATOM | 535 | N | HIS | 67 | 0.130 | 32.350 | 10.763 | 1.00 | 0.36 | 1SG | 536 |
| ATOM | 536 | CA | HIS | 67 | −0.218 | 31.006 | 11.130 | 1.00 | 0.36 | 1SG | 537 |
| ATOM | 537 | ND1 | HIS | 67 | −2.715 | 32.134 | 13.771 | 1.00 | 0.36 | 1SG | 538 |
| ATOM | 538 | CG | HIS | 67 | −1.578 | 31.715 | 13.120 | 1.00 | 0.36 | 1SG | 539 |
| ATOM | 539 | CB | HIS | 67 | −1.576 | 30.933 | 11.841 | 1.00 | 0.36 | 1SG | 540 |
| ATOM | 540 | NE2 | HIS | 67 | −0.991 | 32.840 | 14.985 | 1.00 | 0.36 | 1SG | 541 |
| ATOM | 541 | CD2 | HIS | 67 | −0.533 | 32.155 | 13.874 | 1.00 | 0.36 | 1SG | 542 |
| ATOM | 542 | CE1 | HIS | 67 | −2.307 | 32.801 | 14.880 | 1.00 | 0.36 | 1SG | 543 |
| ATOM | 543 | C | HIS | 67 | −0.295 | 30.124 | 9.924 | 1.00 | 0.36 | 1SG | 544 |
| ATOM | 544 | O | HIS | 67 | −1.352 | 29.578 | 9.616 | 1.00 | 0.36 | 1SG | 545 |
| ATOM | 545 | N | LYS | 68 | 0.835 | 29.932 | 9.224 | 1.00 | 0.45 | 1SG | 546 |
| ATOM | 546 | CA | LYS | 68 | 0.824 | 29.091 | 8.063 | 1.00 | 0.45 | 1SG | 547 |
| ATOM | 547 | CB | LYS | 68 | 2.174 | 29.070 | 7.330 | 1.00 | 0.45 | 1SG | 548 |
| ATOM | 548 | CG | LYS | 68 | 2.550 | 30.411 | 6.699 | 1.00 | 0.45 | 1SG | 549 |
| ATOM | 549 | CD | LYS | 68 | 4.020 | 30.492 | 6.284 | 1.00 | 0.45 | 1SG | 550 |
| ATOM | 550 | CE | LYS | 68 | 4.409 | 31.828 | 5.646 | 1.00 | 0.45 | 1SG | 551 |
| ATOM | 551 | NZ | LYS | 68 | 3.819 | 31.939 | 4.292 | 1.00 | 0.45 | 1SG | 552 |
| ATOM | 552 | C | LYS | 68 | 0.532 | 27.682 | 8.484 | 1.00 | 0.45 | 1SG | 553 |
| ATOM | 553 | O | LYS | 68 | −0.273 | 26.993 | 7.860 | 1.00 | 0.45 | 1SG | 554 |
| ATOM | 554 | N | GLN | 69 | 1.183 | 27.228 | 9.569 | 1.00 | 0.39 | 1SG | 555 |
| ATOM | 555 | CA | GLN | 69 | 1.062 | 25.873 | 10.026 | 1.00 | 0.39 | 1SG | 556 |
| ATOM | 556 | CB | GLN | 69 | 2.057 | 25.530 | 11.147 | 1.00 | 0.39 | 1SG | 557 |
| ATOM | 557 | CG | GLN | 69 | 3.517 | 25.565 | 10.688 | 1.00 | 0.39 | 1SG | 558 |
| ATOM | 558 | CD | GLN | 69 | 4.400 | 25.212 | 11.878 | 1.00 | 0.39 | 1SG | 559 |
| ATOM | 559 | OE1 | GLN | 69 | 5.623 | 25.154 | 11.765 | 1.00 | 0.39 | 1SG | 560 |
| ATOM | 560 | NE2 | GLN | 69 | 3.762 | 24.961 | 13.053 | 1.00 | 0.39 | 1SG | 561 |
| ATOM | 561 | C | GLN | 69 | −0.318 | 25.587 | 10.543 | 1.00 | 0.39 | 1SG | 562 |
| ATOM | 562 | O | GLN | 69 | −0.856 | 24.504 | 10.317 | 1.00 | 0.39 | 1SG | 563 |
| ATOM | 563 | N | LEU | 70 | −0.929 | 26.561 | 11.240 | 1.00 | 0.36 | 1SG | 564 |
| ATOM | 564 | CA | LEU | 70 | −2.190 | 26.373 | 11.905 | 1.00 | 0.36 | 1SG | 565 |
| ATOM | 565 | CB | LEU | 70 | −2.615 | 27.606 | 12.720 | 1.00 | 0.36 | 1SG | 566 |
| ATOM | 566 | CG | LEU | 70 | −1.656 | 27.930 | 13.881 | 1.00 | 0.36 | 1SG | 567 |
| ATOM | 567 | CD2 | LEU | 70 | −2.248 | 28.996 | 14.816 | 1.00 | 0.36 | 1SG | 568 |
| ATOM | 568 | CD1 | LEU | 70 | −0.257 | 28.304 | 13.364 | 1.00 | 0.36 | 1SG | 569 |
| ATOM | 569 | C | LEU | 70 | −3.292 | 26.078 | 10.931 | 1.00 | 0.36 | 1SG | 570 |
| ATOM | 570 | O | LEU | 70 | −4.192 | 25.294 | 11.223 | 1.00 | 0.36 | 1SG | 571 |
| ATOM | 571 | N | ALA | 71 | −3.234 | 26.688 | 9.739 | 1.00 | 0.43 | 1SG | 572 |
| ATOM | 572 | CA | ALA | 71 | −4.249 | 26.641 | 8.721 | 1.00 | 0.43 | 1SG | 573 |
| ATOM | 573 | CB | ALA | 71 | −3.911 | 27.530 | 7.513 | 1.00 | 0.43 | 1SG | 574 |
| ATOM | 574 | C | ALA | 71 | −4.433 | 25.255 | 8.194 | 1.00 | 0.43 | 1SG | 575 |
| ATOM | 575 | O | ALA | 71 | −5.455 | 24.950 | 7.585 | 1.00 | 0.43 | 1SG | 576 |
| ATOM | 576 | N | GLU | 72 | −3.464 | 24.368 | 8.444 | 1.00 | 0.55 | 1SG | 577 |
| ATOM | 577 | CA | GLU | 72 | −3.360 | 23.124 | 7.748 | 1.00 | 0.55 | 1SG | 578 |
| ATOM | 578 | CB | GLU | 72 | −2.279 | 22.213 | 8.354 | 1.00 | 0.55 | 1SG | 579 |
| ATOM | 579 | CG | GLU | 72 | −1.881 | 21.052 | 7.443 | 1.00 | 0.55 | 1SG | 580 |
| ATOM | 580 | CD | GLU | 72 | −0.574 | 20.478 | 7.968 | 1.00 | 0.55 | 1SG | 581 |
| ATOM | 581 | OE1 | GLU | 72 | −0.231 | 20.759 | 9.147 | 1.00 | 0.55 | 1SG | 582 |
| ATOM | 582 | OE2 | GLU | 72 | 0.102 | 19.751 | 7.193 | 1.00 | 0.55 | 1SG | 583 |
| ATOM | 583 | C | GLU | 72 | −4.637 | 22.335 | 7.643 | 1.00 | 0.55 | 1SG | 584 |
| ATOM | 584 | O | GLU | 72 | −4.963 | 21.924 | 6.532 | 1.00 | 0.55 | 1SG | 585 |
| ATOM | 585 | N | ARG | 73 | −5.445 | 22.094 | 8.698 | 1.00 | 0.75 | 1SG | 586 |
| ATOM | 586 | CA | ARG | 73 | −6.461 | 21.157 | 8.294 | 1.00 | 0.75 | 1SG | 587 |
| ATOM | 587 | CB | ARG | 73 | −5.970 | 19.704 | 8.430 | 1.00 | 0.75 | 1SG | 588 |
| ATOM | 588 | CG | ARG | 73 | −4.755 | 19.390 | 7.554 | 1.00 | 0.75 | 1SG | 589 |
| ATOM | 589 | CD | ARG | 73 | −4.076 | 18.062 | 7.886 | 1.00 | 0.75 | 1SG | 590 |
| ATOM | 590 | NE | ARG | 73 | −2.922 | 17.905 | 6.957 | 1.00 | 0.75 | 1SG | 591 |
| ATOM | 591 | CZ | ARG | 73 | −3.064 | 17.184 | 5.804 | 1.00 | 0.75 | 1SG | 592 |
| ATOM | 592 | NH1 | ARG | 73 | −4.254 | 16.580 | 5.524 | 1.00 | 0.75 | 1SG | 593 |
| ATOM | 593 | NH2 | ARG | 73 | −2.018 | 17.072 | 4.936 | 1.00 | 0.75 | 1SG | 594 |
| ATOM | 594 | C | ARG | 73 | −7.741 | 21.229 | 9.072 | 1.00 | 0.75 | 1SG | 595 |
| ATOM | 595 | O | ARG | 73 | −8.642 | 22.015 | 8.783 | 1.00 | 0.75 | 1SG | 596 |
| ATOM | 596 | N | MET | 74 | −7.809 | 20.357 | 10.094 | 1.00 | 0.85 | 1SG | 597 |
| ATOM | 597 | CA | MET | 74 | −8.954 | 19.983 | 10.879 | 1.00 | 0.85 | 1SG | 598 |
| ATOM | 598 | CB | MET | 74 | −8.564 | 19.026 | 12.012 | 1.00 | 0.85 | 1SG | 599 |
| ATOM | 599 | CG | MET | 74 | −9.542 | 19.015 | 13.191 | 1.00 | 0.85 | 1SG | 600 |
| ATOM | 600 | SD | MET | 74 | −11.142 | 18.203 | 12.918 | 1.00 | 0.85 | 1SG | 601 |
| ATOM | 601 | CE | MET | 74 | −11.905 | 18.978 | 14.369 | 1.00 | 0.85 | 1SG | 602 |
| ATOM | 602 | C | MET | 74 | −9.671 | 21.097 | 11.554 | 1.00 | 0.85 | 1SG | 603 |
| ATOM | 603 | O | MET | 74 | −10.901 | 21.117 | 11.532 | 1.00 | 0.85 | 1SG | 604 |
| ATOM | 604 | N | ALA | 75 | −8.947 | 22.051 | 12.158 | 1.00 | 0.71 | 1SG | 605 |
| ATOM | 605 | CA | ALA | 75 | −9.616 | 22.982 | 13.017 | 1.00 | 0.71 | 1SG | 606 |
| ATOM | 606 | CB | ALA | 75 | −8.660 | 24.035 | 13.601 | 1.00 | 0.71 | 1SG | 607 |
| ATOM | 607 | C | ALA | 75 | −10.706 | 23.724 | 12.313 | 1.00 | 0.71 | 1SG | 608 |
| ATOM | 608 | O | ALA | 75 | −11.857 | 23.680 | 12.745 | 1.00 | 0.71 | 1SG | 609 |
| ATOM | 609 | N | ILE | 76 | −10.410 | 24.390 | 11.186 | 1.00 | 0.82 | 1SG | 610 |
| ATOM | 610 | CA | ILE | 76 | −11.470 | 25.194 | 10.663 | 1.00 | 0.82 | 1SG | 611 |
| ATOM | 611 | CB | ILE | 76 | −10.958 | 26.329 | 9.795 | 1.00 | 0.82 | 1SG | 612 |
| ATOM | 612 | CG2 | ILE | 76 | −10.053 | 27.175 | 10.704 | 1.00 | 0.82 | 1SG | 613 |
| ATOM | 613 | CG1 | ILE | 76 | −10.238 | 25.896 | 8.505 | 1.00 | 0.82 | 1SG | 614 |
| ATOM | 614 | CD1 | ILE | 76 | −11.131 | 25.728 | 7.275 | 1.00 | 0.82 | 1SG | 615 |
| ATOM | 615 | C | ILE | 76 | −12.525 | 24.364 | 9.989 | 1.00 | 0.82 | 1SG | 616 |
| ATOM | 616 | O | ILE | 76 | −13.663 | 24.305 | 10.451 | 1.00 | 0.82 | 1SG | 617 |
| ATOM | 617 | N | ARG | 77 | −12.169 | 23.681 | 8.889 | 1.00 | 0.87 | 1SG | 618 |
| ATOM | 618 | CA | ARG | 77 | −13.129 | 22.965 | 8.094 | 1.00 | 0.87 | 1SG | 619 |
| ATOM | 619 | CB | ARG | 77 | −12.604 | 22.607 | 6.695 | 1.00 | 0.87 | 1SG | 620 |
| ATOM | 620 | CG | ARG | 77 | −11.406 | 21.656 | 6.729 | 1.00 | 0.87 | 1SG | 621 |
| ATOM | 621 | CD | ARG | 77 | −11.172 | 20.920 | 5.410 | 1.00 | 0.87 | 1SG | 622 |
| ATOM | 622 | NE | ARG | 77 | −10.385 | 21.801 | 4.509 | 1.00 | 0.87 | 1SG | 623 |
| ATOM | 623 | CZ | ARG | 77 | −9.386 | 21.246 | 3.769 | 1.00 | 0.87 | 1SG | 624 |
| ATOM | 624 | NH1 | ARG | 77 | −9.079 | 19.928 | 3.950 | 1.00 | 0.87 | 1SG | 625 |
| ATOM | 625 | NH2 | ARG | 77 | −8.705 | 22.009 | 2.866 | 1.00 | 0.87 | 1SG | 626 |
| ATOM | 626 | C | ARG | 77 | −13.568 | 21.662 | 8.685 | 1.00 | 0.87 | 1SG | 627 |
| ATOM | 627 | O | ARG | 77 | −14.759 | 21.353 | 8.720 | 1.00 | 0.87 | 1SG | 628 |
| ATOM | 628 | N | GLN | 78 | −12.609 | 20.871 | 9.194 | 1.00 | 0.44 | 1SG | 629 |
| ATOM | 629 | CA | GLN | 78 | −12.886 | 19.506 | 9.533 | 1.00 | 0.44 | 1SG | 630 |
| ATOM | 630 | CB | GLN | 78 | −11.636 | 18.704 | 9.933 | 1.00 | 0.44 | 1SG | 631 |
| ATOM | 631 | CG | GLN | 78 | −11.930 | 17.212 | 10.116 | 1.00 | 0.44 | 1SG | 632 |
| ATOM | 632 | CD | GLN | 78 | −10.613 | 16.446 | 10.150 | 1.00 | 0.44 | 1SG | 633 |
| ATOM | 633 | OE1 | GLN | 78 | −9.690 | 16.791 | 10.885 | 1.00 | 0.44 | 1SG | 634 |
| ATOM | 634 | NE2 | GLN | 78 | −10.522 | 15.371 | 9.320 | 1.00 | 0.44 | 1SG | 635 |
| ATOM | 635 | C | GLN | 78 | −13.923 | 19.399 | 10.601 | 1.00 | 0.44 | 1SG | 636 |
| ATOM | 636 | O | GLN | 78 | −14.679 | 18.428 | 10.618 | 1.00 | 0.44 | 1SG | 637 |
| ATOM | 637 | N | THR | 79 | −13.998 | 20.386 | 11.511 | 1.00 | 0.16 | 1SG | 638 |
| ATOM | 638 | CA | THR | 79 | −14.960 | 20.320 | 12.572 | 1.00 | 0.16 | 1SG | 639 |
| ATOM | 639 | CB | THR | 79 | −14.977 | 21.565 | 13.406 | 1.00 | 0.16 | 1SG | 640 |
| ATOM | 640 | OG1 | THR | 79 | −13.701 | 21.777 | 13.991 | 1.00 | 0.16 | 1SG | 641 |
| ATOM | 641 | CG2 | THR | 79 | −16.041 | 21.410 | 14.503 | 1.00 | 0.16 | 1SG | 642 |
| ATOM | 642 | C | THR | 79 | −16.305 | 20.185 | 11.941 | 1.00 | 0.16 | 1SG | 643 |
| ATOM | 643 | O | THR | 79 | −17.139 | 19.410 | 12.408 | 1.00 | 0.16 | 1SG | 644 |
| ATOM | 644 | N | TRP | 80 | −16.540 | 20.918 | 10.843 | 1.00 | 0.24 | 1SG | 645 |
| ATOM | 645 | CA | TRP | 80 | −17.793 | 20.878 | 10.150 | 1.00 | 0.24 | 1SG | 646 |
| ATOM | 646 | CB | TRP | 80 | −17.852 | 21.854 | 8.961 | 1.00 | 0.24 | 1SG | 647 |
| ATOM | 647 | CG | TRP | 80 | −19.193 | 21.910 | 8.264 | 1.00 | 0.24 | 1SG | 648 |
| ATOM | 648 | CD2 | TRP | 80 | −19.673 | 20.922 | 7.336 | 1.00 | 0.24 | 1SG | 649 |
| ATOM | 649 | CD1 | TRP | 80 | −20.177 | 22.848 | 8.371 | 1.00 | 0.24 | 1SG | 650 |
| ATOM | 650 | NE1 | TRP | 80 | −21.239 | 22.512 | 7.564 | 1.00 | 0.24 | 1SG | 651 |
| ATOM | 651 | CE2 | TRP | 80 | −20.942 | 21.328 | 6.924 | 1.00 | 0.24 | 1SG | 652 |
| ATOM | 652 | CE3 | TRP | 80 | −19.104 | 19.774 | 6.868 | 1.00 | 0.24 | 1SG | 653 |
| ATOM | 653 | CZ2 | TRP | 80 | −21.665 | 20.585 | 6.031 | 1.00 | 0.24 | 1SG | 654 |
| ATOM | 654 | CZ3 | TRP | 80 | −19.834 | 19.028 | 5.970 | 1.00 | 0.24 | 1SG | 655 |
| ATOM | 655 | CH2 | TRP | 80 | −21.090 | 19.425 | 5.560 | 1.00 | 0.24 | 1SG | 656 |
| ATOM | 656 | C | TRP | 80 | −17.976 | 19.510 | 9.587 | 1.00 | 0.24 | 1SG | 657 |
| ATOM | 657 | O | TRP | 80 | −19.104 | 19.020 | 9.510 | 1.00 | 0.24 | 1SG | 658 |
| ATOM | 658 | N | GLY | 81 | −16.853 | 18.859 | 9.208 | 1.00 | 0.56 | 1SG | 659 |
| ATOM | 659 | CA | GLY | 81 | −16.891 | 17.595 | 8.531 | 1.00 | 0.56 | 1SG | 660 |
| ATOM | 660 | C | GLY | 81 | −17.727 | 16.678 | 9.327 | 1.00 | 0.56 | 1SG | 661 |
| ATOM | 661 | O | GLY | 81 | −18.666 | 16.085 | 8.797 | 1.00 | 0.56 | 1SG | 662 |
| ATOM | 662 | N | LYS | 82 | −17.413 | 16.597 | 10.629 | 1.00 | 1.02 | 1SG | 663 |
| ATOM | 663 | CA | LYS | 82 | −18.190 | 15.850 | 11.561 | 1.00 | 1.02 | 1SG | 664 |
| ATOM | 664 | CB | LYS | 82 | −19.540 | 16.524 | 11.839 | 1.00 | 1.02 | 1SG | 665 |
| ATOM | 665 | CG | LYS | 82 | −19.442 | 17.932 | 12.416 | 1.00 | 1.02 | 1SG | 666 |
| ATOM | 666 | CD | LYS | 82 | −18.963 | 17.979 | 13.862 | 1.00 | 1.02 | 1SG | 667 |
| ATOM | 667 | CE | LYS | 82 | −18.993 | 19.394 | 14.434 | 1.00 | 1.02 | 1SG | 668 |
| ATOM | 668 | NZ | LYS | 82 | −18.828 | 19.339 | 15.894 | 1.00 | 1.02 | 1SG | 669 |
| ATOM | 669 | C | LYS | 82 | −18.543 | 14.522 | 10.995 | 1.00 | 1.02 | 1SG | 670 |
| ATOM | 670 | O | LYS | 82 | −19.633 | 14.400 | 10.432 | 1.00 | 1.02 | 1SG | 671 |
| ATOM | 671 | N | GLU | 83 | −17.624 | 13.532 | 11.036 | 1.00 | 1.19 | 1SG | 672 |
| ATOM | 672 | CA | GLU | 83 | −18.159 | 12.240 | 10.749 | 1.00 | 1.19 | 1SG | 673 |
| ATOM | 673 | CB | GLU | 83 | −17.233 | 11.048 | 10.980 | 1.00 | 1.19 | 1SG | 674 |
| ATOM | 674 | CG | GLU | 83 | −18.055 | 9.775 | 11.240 | 1.00 | 1.19 | 1SG | 675 |
| ATOM | 675 | CD | GLU | 83 | −18.930 | 9.437 | 10.034 | 1.00 | 1.19 | 1SG | 676 |
| ATOM | 676 | OE1 | GLU | 83 | −18.987 | 10.247 | 9.071 | 1.00 | 1.19 | 1SG | 677 |
| ATOM | 677 | OE2 | GLU | 83 | −19.549 | 8.339 | 10.060 | 1.00 | 1.19 | 1SG | 678 |
| ATOM | 678 | C | GLU | 83 | −19.113 | 12.158 | 11.852 | 1.00 | 1.19 | 1SG | 679 |
| ATOM | 679 | O | GLU | 83 | −20.273 | 11.801 | 11.684 | 1.00 | 1.19 | 1SG | 680 |
| ATOM | 680 | N | ARG | 84 | −18.581 | 12.538 | 13.017 | 1.00 | 0.97 | 1SG | 681 |
| ATOM | 681 | CA | ARG | 84 | −19.338 | 12.800 | 14.182 | 1.00 | 0.97 | 1SG | 682 |
| ATOM | 682 | CB | ARG | 84 | −19.949 | 11.577 | 14.881 | 1.00 | 0.97 | 1SG | 683 |
| ATOM | 683 | CG | ARG | 84 | −19.129 | 10.354 | 15.283 | 1.00 | 0.97 | 1SG | 684 |
| ATOM | 684 | CD | ARG | 84 | −20.103 | 9.326 | 15.875 | 1.00 | 0.97 | 1SG | 685 |
| ATOM | 685 | NE | ARG | 84 | −19.480 | 7.978 | 15.939 | 1.00 | 0.97 | 1SG | 686 |
| ATOM | 686 | CZ | ARG | 84 | −19.478 | 7.172 | 14.837 | 1.00 | 0.97 | 1SG | 687 |
| ATOM | 687 | NH1 | ARG | 84 | −19.700 | 7.685 | 13.594 | 1.00 | 0.97 | 1SG | 688 |
| ATOM | 688 | NH2 | ARG | 84 | −19.190 | 5.849 | 14.985 | 1.00 | 0.97 | 1SG | 689 |
| ATOM | 689 | C | ARG | 84 | −18.446 | 13.546 | 15.089 | 1.00 | 0.97 | 1SG | 690 |
| ATOM | 690 | O | ARG | 84 | −18.148 | 13.082 | 16.189 | 1.00 | 0.97 | 1SG | 691 |
| ATOM | 691 | N | MET | 85 | −17.965 | 14.724 | 14.628 | 1.00 | 0.74 | 1SG | 692 |
| ATOM | 692 | CA | MET | 85 | −17.152 | 15.430 | 15.557 | 1.00 | 0.74 | 1SG | 693 |
| ATOM | 693 | CB | MET | 85 | −16.546 | 16.745 | 15.045 | 1.00 | 0.74 | 1SG | 694 |
| ATOM | 694 | CG | MET | 85 | −15.534 | 17.309 | 16.045 | 1.00 | 0.74 | 1SG | 695 |
| ATOM | 695 | SD | MET | 85 | −14.732 | 18.858 | 15.559 | 1.00 | 0.74 | 1SG | 696 |
| ATOM | 696 | CE | MET | 85 | −13.467 | 18.759 | 16.857 | 1.00 | 0.74 | 1SG | 697 |
| ATOM | 697 | C | MET | 85 | −18.061 | 15.765 | 16.679 | 1.00 | 0.74 | 1SG | 698 |
| ATOM | 698 | O | MET | 85 | −17.875 | 15.279 | 17.787 | 1.00 | 0.74 | 1SG | 699 |
| ATOM | 699 | N | VAL | 86 | −19.135 | 16.530 | 16.389 | 1.00 | 0.61 | 1SG | 700 |
| ATOM | 700 | CA | VAL | 86 | −20.071 | 16.830 | 17.429 | 1.00 | 0.61 | 1SG | 701 |
| ATOM | 701 | CB | VAL | 86 | −19.690 | 17.976 | 18.300 | 1.00 | 0.61 | 1SG | 702 |
| ATOM | 702 | CG1 | VAL | 86 | −20.824 | 18.138 | 19.322 | 1.00 | 0.61 | 1SG | 703 |
| ATOM | 703 | CG2 | VAL | 86 | −18.279 | 17.752 | 18.867 | 1.00 | 0.61 | 1SG | 704 |
| ATOM | 704 | C | VAL | 86 | −21.335 | 17.279 | 16.792 | 1.00 | 0.61 | 1SG | 705 |
| ATOM | 705 | O | VAL | 86 | −21.405 | 18.392 | 16.276 | 1.00 | 0.61 | 1SG | 706 |
| ATOM | 706 | N | LYS | 87 | −22.376 | 16.425 | 16.812 | 1.00 | 0.60 | 1SG | 707 |
| ATOM | 707 | CA | LYS | 87 | −23.661 | 16.775 | 16.267 | 1.00 | 0.60 | 1SG | 708 |
| ATOM | 708 | CB | LYS | 87 | −24.250 | 18.062 | 16.871 | 1.00 | 0.60 | 1SG | 709 |
| ATOM | 709 | CG | LYS | 87 | −24.677 | 17.936 | 18.335 | 1.00 | 0.60 | 1SG | 710 |
| ATOM | 710 | CD | LYS | 87 | −24.999 | 19.281 | 18.994 | 1.00 | 0.60 | 1SG | 711 |
| ATOM | 711 | CE | LYS | 87 | −26.162 | 20.030 | 18.336 | 1.00 | 0.60 | 1SG | 712 |
| ATOM | 712 | NZ | LYS | 87 | −25.754 | 20.547 | 17.009 | 1.00 | 0.60 | 1SG | 713 |
| ATOM | 713 | C | LYS | 87 | −23.588 | 16.988 | 14.785 | 1.00 | 0.60 | 1SG | 714 |
| ATOM | 714 | O | LYS | 87 | −24.621 | 17.036 | 14.119 | 1.00 | 0.60 | 1SG | 715 |
| ATOM | 715 | N | GLY | 88 | −22.377 | 17.109 | 14.212 | 1.00 | 0.66 | 1SG | 716 |
| ATOM | 716 | CA | GLY | 88 | −22.298 | 17.385 | 12.820 | 1.00 | 0.66 | 1SG | 717 |
| ATOM | 717 | C | GLY | 88 | −22.937 | 16.263 | 12.093 | 1.00 | 0.66 | 1SG | 718 |
| ATOM | 718 | O | GLY | 88 | −23.810 | 16.470 | 11.253 | 1.00 | 0.66 | 1SG | 719 |
| ATOM | 719 | N | LYS | 89 | −22.543 | 15.020 | 12.409 | 1.00 | 0.73 | 1SG | 720 |
| ATOM | 720 | CA | LYS | 89 | −23.155 | 13.977 | 11.655 | 1.00 | 0.73 | 1SG | 721 |
| ATOM | 721 | CB | LYS | 89 | −22.243 | 13.372 | 10.571 | 1.00 | 0.73 | 1SG | 722 |
| ATOM | 722 | CG | LYS | 89 | −22.930 | 12.258 | 9.774 | 1.00 | 0.73 | 1SG | 723 |
| ATOM | 723 | CD | LYS | 89 | −22.220 | 11.887 | 8.471 | 1.00 | 0.73 | 1SG | 724 |
| ATOM | 724 | CE | LYS | 89 | −22.925 | 10.778 | 7.685 | 1.00 | 0.73 | 1SG | 725 |
| ATOM | 725 | NZ | LYS | 89 | −24.227 | 11.265 | 7.171 | 1.00 | 0.73 | 1SG | 726 |
| ATOM | 726 | C | LYS | 89 | −23.549 | 12.871 | 12.571 | 1.00 | 0.73 | 1SG | 727 |
| ATOM | 727 | O | LYS | 89 | −24.668 | 12.824 | 13.072 | 1.00 | 0.73 | 1SG | 728 |
| ATOM | 728 | N | GLN | 90 | −22.602 | 11.954 | 12.809 | 1.00 | 0.62 | 1SG | 729 |
| ATOM | 729 | CA | GLN | 90 | −22.833 | 10.747 | 13.537 | 1.00 | 0.62 | 1SG | 730 |
| ATOM | 730 | CB | GLN | 90 | −21.688 | 9.733 | 13.405 | 1.00 | 0.62 | 1SG | 731 |
| ATOM | 731 | CG | GLN | 90 | −21.394 | 9.317 | 11.955 | 1.00 | 0.62 | 1SG | 732 |
| ATOM | 732 | CD | GLN | 90 | −22.647 | 8.741 | 11.314 | 1.00 | 0.62 | 1SG | 733 |
| ATOM | 733 | OE1 | GLN | 90 | −23.719 | 9.345 | 11.347 | 1.00 | 0.62 | 1SG | 734 |
| ATOM | 734 | NE2 | GLN | 90 | −22.500 | 7.540 | 10.693 | 1.00 | 0.62 | 1SG | 735 |
| ATOM | 735 | C | GLN | 90 | −23.146 | 11.031 | 14.973 | 1.00 | 0.62 | 1SG | 736 |
| ATOM | 736 | O | GLN | 90 | −23.858 | 10.259 | 15.612 | 1.00 | 0.62 | 1SG | 737 |
| ATOM | 737 | N | LEU | 91 | −22.608 | 12.115 | 15.559 | 1.00 | 0.42 | 1SG | 738 |
| ATOM | 738 | CA | LEU | 91 | −23.000 | 12.330 | 16.915 | 1.00 | 0.42 | 1SG | 739 |
| ATOM | 739 | CB | LEU | 91 | −22.292 | 13.498 | 17.612 | 1.00 | 0.42 | 1SG | 740 |
| ATOM | 740 | CG | LEU | 91 | −20.811 | 13.187 | 17.878 | 1.00 | 0.42 | 1SG | 741 |
| ATOM | 741 | CD2 | LEU | 91 | −20.634 | 11.748 | 18.390 | 1.00 | 0.42 | 1SG | 742 |
| ATOM | 742 | CD1 | LEU | 91 | −20.187 | 14.229 | 18.814 | 1.00 | 0.42 | 1SG | 743 |
| ATOM | 743 | C | LEU | 91 | −24.460 | 12.604 | 16.885 | 1.00 | 0.42 | 1SG | 744 |
| ATOM | 744 | O | LEU | 91 | −25.214 | 12.123 | 17.729 | 1.00 | 0.42 | 1SG | 745 |
| ATOM | 745 | N | LYS | 92 | −24.896 | 13.383 | 15.883 | 1.00 | 0.60 | 1SG | 746 |
| ATOM | 746 | CA | LYS | 92 | −26.292 | 13.651 | 15.774 | 1.00 | 0.60 | 1SG | 747 |
| ATOM | 747 | CB | LYS | 92 | −26.644 | 14.672 | 14.680 | 1.00 | 0.60 | 1SG | 748 |
| ATOM | 748 | CG | LYS | 92 | −28.136 | 15.011 | 14.655 | 1.00 | 0.60 | 1SG | 749 |
| ATOM | 749 | CD | LYS | 92 | −28.483 | 16.250 | 13.826 | 1.00 | 0.60 | 1SG | 750 |
| ATOM | 750 | CE | LYS | 92 | −28.715 | 15.952 | 12.343 | 1.00 | 0.60 | 1SG | 751 |
| ATOM | 751 | NZ | LYS | 92 | −29.065 | 17.199 | 11.627 | 1.00 | 0.60 | 1SG | 752 |
| ATOM | 752 | C | LYS | 92 | −26.989 | 12.370 | 15.436 | 1.00 | 0.60 | 1SG | 753 |
| ATOM | 753 | O | LYS | 92 | −28.072 | 12.097 | 15.950 | 1.00 | 0.60 | 1SG | 754 |
| ATOM | 754 | N | THR | 93 | −26.378 | 11.534 | 14.568 | 1.00 | 1.03 | 1SG | 755 |
| ATOM | 755 | CA | THR | 93 | −27.078 | 10.356 | 14.150 | 1.00 | 1.03 | 1SG | 756 |
| ATOM | 756 | CB | THR | 93 | −26.371 | 9.541 | 13.089 | 1.00 | 1.03 | 1SG | 757 |
| ATOM | 757 | OG1 | THR | 93 | −27.265 | 8.573 | 12.562 | 1.00 | 1.03 | 1SG | 758 |
| ATOM | 758 | CG2 | THR | 93 | −25.124 | 8.842 | 13.650 | 1.00 | 1.03 | 1SG | 759 |
| ATOM | 759 | C | THR | 93 | −27.387 | 9.505 | 15.344 | 1.00 | 1.03 | 1SG | 760 |
| ATOM | 760 | O | THR | 93 | −28.561 | 9.222 | 15.575 | 1.00 | 1.03 | 1SG | 761 |
| ATOM | 761 | N | PHE | 94 | −26.374 | 9.115 | 16.158 | 1.00 | 1.22 | 1SG | 762 |
| ATOM | 762 | CA | PHE | 94 | −26.669 | 8.347 | 17.340 | 1.00 | 1.22 | 1SG | 763 |
| ATOM | 763 | CB | PHE | 94 | −27.677 | 7.186 | 17.153 | 1.00 | 1.22 | 1SG | 764 |
| ATOM | 764 | CG | PHE | 94 | −27.208 | 6.171 | 16.174 | 1.00 | 1.22 | 1SG | 765 |
| ATOM | 765 | CD1 | PHE | 94 | −27.377 | 6.372 | 14.822 | 1.00 | 1.22 | 1SG | 766 |
| ATOM | 766 | CD2 | PHE | 94 | −26.611 | 5.014 | 16.611 | 1.00 | 1.22 | 1SG | 767 |
| ATOM | 767 | CE1 | PHE | 94 | −26.951 | 5.431 | 13.916 | 1.00 | 1.22 | 1SG | 768 |
| ATOM | 768 | CE2 | PHE | 94 | −26.186 | 4.069 | 15.710 | 1.00 | 1.22 | 1SG | 769 |
| ATOM | 769 | CZ | PHE | 94 | −26.353 | 4.276 | 14.360 | 1.00 | 1.22 | 1SG | 770 |
| ATOM | 770 | C | PHE | 94 | −25.428 | 7.828 | 18.012 | 1.00 | 1.22 | 1SG | 771 |
| ATOM | 771 | O | PHE | 94 | −25.135 | 8.238 | 19.134 | 1.00 | 1.22 | 1SG | 772 |
| ATOM | 772 | N | PHE | 95 | −24.659 | 6.921 | 17.364 | 1.00 | 0.99 | 1SG | 773 |
| ATOM | 773 | CA | PHE | 95 | −23.518 | 6.345 | 18.032 | 1.00 | 0.99 | 1SG | 774 |
| ATOM | 774 | CB | PHE | 95 | −22.774 | 5.238 | 17.264 | 1.00 | 0.99 | 1SG | 775 |
| ATOM | 775 | CG | PHE | 95 | −22.844 | 5.543 | 15.815 | 1.00 | 0.99 | 1SG | 776 |
| ATOM | 776 | CD1 | PHE | 95 | −22.348 | 6.704 | 15.293 | 1.00 | 0.99 | 1SG | 777 |
| ATOM | 777 | CD2 | PHE | 95 | −23.401 | 4.623 | 14.962 | 1.00 | 0.99 | 1SG | 778 |
| ATOM | 778 | CE1 | PHE | 95 | −22.432 | 6.938 | 13.943 | 1.00 | 0.99 | 1SG | 779 |
| ATOM | 779 | CE2 | PHE | 95 | −23.485 | 4.856 | 13.612 | 1.00 | 0.99 | 1SG | 780 |
| ATOM | 780 | CZ | PHE | 95 | −22.998 | 6.026 | 13.092 | 1.00 | 0.99 | 1SG | 781 |
| ATOM | 781 | C | PHE | 95 | −22.590 | 7.366 | 18.610 | 1.00 | 0.99 | 1SG | 782 |
| ATOM | 782 | O | PHE | 95 | −22.113 | 8.293 | 17.956 | 1.00 | 0.99 | 1SG | 783 |
| ATOM | 783 | N | LEU | 96 | −22.365 | 7.170 | 19.924 | 1.00 | 0.70 | 1SG | 784 |
| ATOM | 784 | CA | LEU | 96 | −21.681 | 7.996 | 20.880 | 1.00 | 0.70 | 1SG | 785 |
| ATOM | 785 | CB | LEU | 96 | −21.886 | 7.488 | 22.321 | 1.00 | 0.70 | 1SG | 786 |
| ATOM | 786 | CG | LEU | 96 | −23.357 | 7.424 | 22.789 | 1.00 | 0.70 | 1SG | 787 |
| ATOM | 787 | CD2 | LEU | 96 | −24.173 | 6.419 | 21.960 | 1.00 | 0.70 | 1SG | 788 |
| ATOM | 788 | CD1 | LEU | 96 | −24.009 | 8.814 | 22.827 | 1.00 | 0.70 | 1SG | 789 |
| ATOM | 789 | C | LEU | 96 | −20.191 | 8.107 | 20.714 | 1.00 | 0.70 | 1SG | 790 |
| ATOM | 790 | O | LEU | 96 | −19.636 | 9.188 | 20.907 | 1.00 | 0.70 | 1SG | 791 |
| ATOM | 791 | N | LEU | 97 | −19.488 | 7.013 | 20.363 | 1.00 | 0.51 | 1SG | 792 |
| ATOM | 792 | CA | LEU | 97 | −18.051 | 7.043 | 20.497 | 1.00 | 0.51 | 1SG | 793 |
| ATOM | 793 | CB | LEU | 97 | −17.510 | 5.819 | 21.260 | 1.00 | 0.51 | 1SG | 794 |
| ATOM | 794 | CG | LEU | 97 | −17.825 | 5.746 | 22.769 | 1.00 | 0.51 | 1SG | 795 |
| ATOM | 795 | CD2 | LEU | 97 | −19.263 | 6.197 | 23.060 | 1.00 | 0.51 | 1SG | 796 |
| ATOM | 796 | CD1 | LEU | 97 | −16.806 | 6.521 | 23.612 | 1.00 | 0.51 | 1SG | 797 |
| ATOM | 797 | C | LEU | 97 | −17.354 | 6.998 | 19.182 | 1.00 | 0.51 | 1SG | 798 |
| ATOM | 798 | O | LEU | 97 | −17.968 | 6.811 | 18.133 | 1.00 | 0.51 | 1SG | 799 |
| ATOM | 799 | N | GLY | 98 | −16.020 | 7.220 | 19.233 | 1.00 | 0.45 | 1SG | 800 |
| ATOM | 800 | CA | GLY | 98 | −15.195 | 7.089 | 18.069 | 1.00 | 0.45 | 1SG | 801 |
| ATOM | 801 | C | GLY | 98 | −13.871 | 7.748 | 18.297 | 1.00 | 0.45 | 1SG | 802 |
| ATOM | 802 | O | GLY | 98 | −13.651 | 8.412 | 19.308 | 1.00 | 0.45 | 1SG | 803 |
| ATOM | 803 | N | THR | 99 | −12.948 | 7.561 | 17.328 | 1.00 | 0.65 | 1SG | 804 |
| ATOM | 804 | CA | THR | 99 | −11.651 | 8.169 | 17.372 | 1.00 | 0.65 | 1SG | 805 |
| ATOM | 805 | CB | THR | 99 | −10.541 | 7.278 | 16.888 | 1.00 | 0.65 | 1SG | 806 |
| ATOM | 806 | OG1 | THR | 99 | −10.732 | 6.935 | 15.523 | 1.00 | 0.65 | 1SG | 807 |
| ATOM | 807 | CG2 | THR | 99 | −10.515 | 6.008 | 17.755 | 1.00 | 0.65 | 1SG | 808 |
| ATOM | 808 | C | THR | 99 | −11.729 | 9.345 | 16.451 | 1.00 | 0.65 | 1SG | 809 |
| ATOM | 809 | O | THR | 99 | −12.365 | 9.276 | 15.401 | 1.00 | 0.65 | 1SG | 810 |
| ATOM | 810 | N | THR | 100 | −11.068 | 10.461 | 16.814 | 1.00 | 0.80 | 1SG | 811 |
| ATOM | 811 | CA | THR | 100 | −11.244 | 11.653 | 16.034 | 1.00 | 0.80 | 1SG | 812 |
| ATOM | 812 | CB | THR | 100 | −11.839 | 12.783 | 16.829 | 1.00 | 0.80 | 1SG | 813 |
| ATOM | 813 | OG1 | THR | 100 | −12.002 | 13.941 | 16.022 | 1.00 | 0.80 | 1SG | 814 |
| ATOM | 814 | CG2 | THR | 100 | −10.929 | 13.092 | 18.026 | 1.00 | 0.80 | 1SG | 815 |
| ATOM | 815 | C | THR | 100 | −9.954 | 12.139 | 15.473 | 1.00 | 0.80 | 1SG | 816 |
| ATOM | 816 | O | THR | 100 | −8.866 | 11.744 | 15.880 | 1.00 | 0.80 | 1SG | 817 |
| ATOM | 817 | N | SER | 101 | −10.077 | 12.999 | 14.443 | 1.00 | 0.78 | 1SG | 818 |
| ATOM | 818 | CA | SER | 101 | −8.941 | 13.635 | 13.861 | 1.00 | 0.78 | 1SG | 819 |
| ATOM | 819 | CB | SER | 101 | −8.940 | 13.635 | 12.320 | 1.00 | 0.78 | 1SG | 820 |
| ATOM | 820 | OG | SER | 101 | −8.859 | 12.302 | 11.833 | 1.00 | 0.78 | 1SG | 821 |
| ATOM | 821 | C | SER | 101 | −9.038 | 15.064 | 14.269 | 1.00 | 0.78 | 1SG | 822 |
| ATOM | 822 | O | SER | 101 | −9.008 | 15.957 | 13.423 | 1.00 | 0.78 | 1SG | 823 |
| ATOM | 823 | N | SER | 102 | −9.163 | 15.315 | 15.586 | 1.00 | 0.70 | 1SG | 824 |
| ATOM | 824 | CA | SER | 102 | −9.205 | 16.663 | 16.067 | 1.00 | 0.70 | 1SG | 825 |
| ATOM | 825 | CB | SER | 102 | −10.628 | 17.185 | 16.334 | 1.00 | 0.70 | 1SG | 826 |
| ATOM | 826 | OG | SER | 102 | −11.245 | 16.412 | 17.352 | 1.00 | 0.70 | 1SG | 827 |
| ATOM | 827 | C | SER | 102 | −8.472 | 16.669 | 17.368 | 1.00 | 0.70 | 1SG | 828 |
| ATOM | 828 | O | SER | 102 | −8.875 | 16.002 | 18.318 | 1.00 | 0.70 | 1SG | 829 |
| ATOM | 829 | N | ALA | 103 | −7.359 | 17.423 | 17.436 | 1.00 | 0.61 | 1SG | 830 |
| ATOM | 830 | CA | ALA | 103 | −6.562 | 17.506 | 18.620 | 1.00 | 0.61 | 1SG | 831 |
| ATOM | 831 | CB | ALA | 103 | −5.673 | 16.274 | 18.850 | 1.00 | 0.61 | 1SG | 832 |
| ATOM | 832 | C | ALA | 103 | −5.647 | 18.668 | 18.428 | 1.00 | 0.61 | 1SG | 833 |
| ATOM | 833 | O | ALA | 103 | −5.867 | 19.512 | 17.562 | 1.00 | 0.61 | 1SG | 834 |
| ATOM | 834 | N | ALA | 104 | −4.580 | 18.736 | 19.242 | 1.00 | 0.57 | 1SG | 835 |
| ATOM | 835 | CA | ALA | 104 | −3.670 | 19.836 | 19.137 | 1.00 | 0.57 | 1SG | 836 |
| ATOM | 836 | CB | ALA | 104 | −2.497 | 19.743 | 20.127 | 1.00 | 0.57 | 1SG | 837 |
| ATOM | 837 | C | ALA | 104 | −3.096 | 19.817 | 17.760 | 1.00 | 0.57 | 1SG | 838 |
| ATOM | 838 | O | ALA | 104 | −3.027 | 20.847 | 17.091 | 1.00 | 0.57 | 1SG | 839 |
| ATOM | 839 | N | GLU | 105 | −2.683 | 18.627 | 17.288 | 1.00 | 0.66 | 1SG | 840 |
| ATOM | 840 | CA | GLU | 105 | −2.110 | 18.557 | 15.981 | 1.00 | 0.66 | 1SG | 841 |
| ATOM | 841 | CB | GLU | 105 | −1.634 | 17.145 | 15.598 | 1.00 | 0.66 | 1SG | 842 |
| ATOM | 842 | CG | GLU | 105 | −0.449 | 16.656 | 16.434 | 1.00 | 0.66 | 1SG | 843 |
| ATOM | 843 | CD | GLU | 105 | −0.081 | 15.255 | 15.967 | 1.00 | 0.66 | 1SG | 844 |
| ATOM | 844 | OE1 | GLU | 105 | −0.787 | 14.728 | 15.066 | 1.00 | 0.66 | 1SG | 845 |
| ATOM | 845 | OE2 | GLU | 105 | 0.908 | 14.692 | 16.508 | 1.00 | 0.66 | 1SG | 846 |
| ATOM | 846 | C | GLU | 105 | −3.171 | 18.969 | 15.018 | 1.00 | 0.66 | 1SG | 847 |
| ATOM | 847 | O | GLU | 105 | −4.332 | 18.583 | 15.150 | 1.00 | 0.66 | 1SG | 848 |
| ATOM | 848 | N | THR | 106 | −2.795 | 19.795 | 14.027 | 1.00 | 0.75 | 1SG | 849 |
| ATOM | 849 | CA | THR | 106 | −3.750 | 20.248 | 13.063 | 1.00 | 0.75 | 1SG | 850 |
| ATOM | 850 | CB | THR | 106 | −3.195 | 21.276 | 12.120 | 1.00 | 0.75 | 1SG | 851 |
| ATOM | 851 | OG1 | THR | 106 | −4.228 | 21.799 | 11.299 | 1.00 | 0.75 | 1SG | 852 |
| ATOM | 852 | CG2 | THR | 106 | −2.096 | 20.633 | 11.258 | 1.00 | 0.75 | 1SG | 853 |
| ATOM | 853 | C | THR | 106 | −4.181 | 19.060 | 12.272 | 1.00 | 0.75 | 1SG | 854 |
| ATOM | 854 | O | THR | 106 | −5.343 | 18.944 | 11.884 | 1.00 | 0.75 | 1SG | 855 |
| ATOM | 855 | N | LYS | 107 | −3.228 | 18.155 | 11.991 | 1.00 | 0.72 | 1SG | 856 |
| ATOM | 856 | CA | LYS | 107 | −3.535 | 16.980 | 11.238 | 1.00 | 0.72 | 1SG | 857 |
| ATOM | 857 | CB | LYS | 107 | −2.280 | 16.319 | 10.655 | 1.00 | 0.72 | 1SG | 858 |
| ATOM | 858 | CG | LYS | 107 | −1.738 | 17.024 | 9.413 | 1.00 | 0.72 | 1SG | 859 |
| ATOM | 859 | CD | LYS | 107 | −0.478 | 16.368 | 8.840 | 1.00 | 0.72 | 1SG | 860 |
| ATOM | 860 | CE | LYS | 107 | 0.038 | 17.027 | 7.560 | 1.00 | 0.72 | 1SG | 861 |
| ATOM | 861 | NZ | LYS | 107 | 1.201 | 16.280 | 7.029 | 1.00 | 0.72 | 1SG | 862 |
| ATOM | 862 | C | LYS | 107 | −4.294 | 15.989 | 12.077 | 1.00 | 0.72 | 1SG | 863 |
| ATOM | 863 | O | LYS | 107 | −5.215 | 15.340 | 11.585 | 1.00 | 0.72 | 1SG | 864 |
| ATOM | 864 | N | GLU | 108 | −3.906 | 15.825 | 13.357 | 1.00 | 0.70 | 1SG | 865 |
| ATOM | 865 | CA | GLU | 108 | −4.599 | 14.958 | 14.274 | 1.00 | 0.70 | 1SG | 866 |
| ATOM | 866 | CB | GLU | 108 | −6.105 | 15.179 | 14.341 | 1.00 | 0.70 | 1SG | 867 |
| ATOM | 867 | CG | GLU | 108 | −6.496 | 16.528 | 14.920 | 1.00 | 0.70 | 1SG | 868 |
| ATOM | 868 | CD | GLU | 108 | −6.685 | 17.492 | 13.772 | 1.00 | 0.70 | 1SG | 869 |
| ATOM | 869 | OE1 | GLU | 108 | −6.813 | 17.011 | 12.614 | 1.00 | 0.70 | 1SG | 870 |
| ATOM | 870 | OE2 | GLU | 108 | −6.717 | 18.724 | 14.035 | 1.00 | 0.70 | 1SG | 871 |
| ATOM | 871 | C | GLU | 108 | −4.402 | 13.528 | 13.901 | 1.00 | 0.70 | 1SG | 872 |
| ATOM | 872 | O | GLU | 108 | −3.323 | 13.142 | 13.459 | 1.00 | 0.70 | 1SG | 873 |
| ATOM | 873 | N | VAL | 109 | −5.451 | 12.685 | 14.081 | 1.00 | 0.59 | 1SG | 874 |
| ATOM | 874 | CA | VAL | 109 | −5.237 | 11.317 | 13.720 | 1.00 | 0.59 | 1SG | 875 |
| ATOM | 875 | CB | VAL | 109 | −6.245 | 10.321 | 14.257 | 1.00 | 0.59 | 1SG | 876 |
| ATOM | 876 | CG1 | VAL | 109 | −7.653 | 10.609 | 13.714 | 1.00 | 0.59 | 1SG | 877 |
| ATOM | 877 | CG2 | VAL | 109 | −5.741 | 8.902 | 13.927 | 1.00 | 0.59 | 1SG | 878 |
| ATOM | 878 | C | VAL | 109 | −5.205 | 11.313 | 12.231 | 1.00 | 0.59 | 1SG | 879 |
| ATOM | 879 | O | VAL | 109 | −6.219 | 11.355 | 11.534 | 1.00 | 0.59 | 1SG | 880 |
| ATOM | 880 | N | ASP | 110 | −3.978 | 11.318 | 11.696 | 1.00 | 0.49 | 1SG | 881 |
| ATOM | 881 | CA | ASP | 110 | −3.826 | 11.358 | 10.284 | 1.00 | 0.49 | 1SG | 882 |
| ATOM | 882 | CB | ASP | 110 | −2.453 | 11.841 | 9.810 | 1.00 | 0.49 | 1SG | 883 |
| ATOM | 883 | CG | ASP | 110 | −2.369 | 13.303 | 10.145 | 1.00 | 0.49 | 1SG | 884 |
| ATOM | 884 | OD1 | ASP | 110 | −3.403 | 13.837 | 10.628 | 1.00 | 0.49 | 1SG | 885 |
| ATOM | 885 | OD2 | ASP | 110 | −1.277 | 13.891 | 9.926 | 1.00 | 0.49 | 1SG | 886 |
| ATOM | 886 | C | ASP | 110 | −3.904 | 9.973 | 9.801 | 1.00 | 0.49 | 1SG | 887 |
| ATOM | 887 | O | ASP | 110 | −3.821 | 9.018 | 10.571 | 1.00 | 0.49 | 1SG | 888 |
| ATOM | 888 | N | GLN | 111 | −4.108 | 9.851 | 8.486 | 1.00 | 0.63 | 1SG | 889 |
| ATOM | 889 | CA | GLN | 111 | −4.052 | 8.558 | 7.907 | 1.00 | 0.63 | 1SG | 890 |
| ATOM | 890 | CB | GLN | 111 | −4.457 | 8.499 | 6.443 | 1.00 | 0.63 | 1SG | 891 |
| ATOM | 891 | CG | GLN | 111 | −4.339 | 7.064 | 5.970 | 1.00 | 0.63 | 1SG | 892 |
| ATOM | 892 | CD | GLN | 111 | −5.152 | 6.203 | 6.921 | 1.00 | 0.63 | 1SG | 893 |
| ATOM | 893 | OE1 | GLN | 111 | −4.614 | 5.611 | 7.855 | 1.00 | 0.63 | 1SG | 894 |
| ATOM | 894 | NE2 | GLN | 111 | −6.485 | 6.123 | 6.672 | 1.00 | 0.63 | 1SG | 895 |
| ATOM | 895 | C | GLN | 111 | −2.622 | 8.140 | 7.976 | 1.00 | 0.63 | 1SG | 896 |
| ATOM | 896 | O | GLN | 111 | −2.316 | 6.951 | 8.028 | 1.00 | 0.63 | 1SG | 897 |
| ATOM | 897 | N | GLU | 112 | −1.712 | 9.135 | 7.964 | 1.00 | 0.55 | 1SG | 898 |
| ATOM | 898 | CA | GLU | 112 | −0.300 | 8.892 | 7.966 | 1.00 | 0.55 | 1SG | 899 |
| ATOM | 899 | CB | GLU | 112 | 0.576 | 10.157 | 7.850 | 1.00 | 0.55 | 1SG | 900 |
| ATOM | 900 | CG | GLU | 112 | 0.480 | 11.127 | 9.029 | 1.00 | 0.55 | 1SG | 901 |
| ATOM | 901 | CD | GLU | 112 | 1.554 | 12.188 | 8.827 | 1.00 | 0.55 | 1SG | 902 |
| ATOM | 902 | OE1 | GLU | 112 | 2.703 | 11.804 | 8.479 | 1.00 | 0.55 | 1SG | 903 |
| ATOM | 903 | OE2 | GLU | 112 | 1.241 | 13.395 | 9.010 | 1.00 | 0.55 | 1SG | 904 |
| ATOM | 904 | C | GLU | 112 | 0.098 | 8.178 | 9.220 | 1.00 | 0.55 | 1SG | 905 |
| ATOM | 905 | O | GLU | 112 | 1.031 | 7.377 | 9.198 | 1.00 | 0.55 | 1SG | 906 |
| ATOM | 906 | N | SER | 113 | −0.569 | 8.454 | 10.356 | 1.00 | 0.37 | 1SG | 907 |
| ATOM | 907 | CA | SER | 113 | −0.174 | 7.784 | 11.565 | 1.00 | 0.37 | 1SG | 908 |
| ATOM | 908 | CB | SER | 113 | −0.814 | 8.370 | 12.833 | 1.00 | 0.37 | 1SG | 909 |
| ATOM | 909 | OG | SER | 113 | −2.212 | 8.118 | 12.837 | 1.00 | 0.37 | 1SG | 910 |
| ATOM | 910 | C | SER | 113 | −0.604 | 6.357 | 11.461 | 1.00 | 0.37 | 1SG | 911 |
| ATOM | 911 | O | SER | 113 | −1.729 | 6.066 | 11.057 | 1.00 | 0.37 | 1SG | 912 |
| ATOM | 912 | N | GLN | 114 | 0.289 | 5.411 | 11.818 | 1.00 | 0.67 | 1SG | 913 |
| ATOM | 913 | CA | GLN | 114 | −0.119 | 4.047 | 11.716 | 1.00 | 0.67 | 1SG | 914 |
| ATOM | 914 | CB | GLN | 114 | 0.874 | 3.131 | 10.979 | 1.00 | 0.67 | 1SG | 915 |
| ATOM | 915 | CG | GLN | 114 | 2.220 | 2.976 | 11.679 | 1.00 | 0.67 | 1SG | 916 |
| ATOM | 916 | CD | GLN | 114 | 3.013 | 1.938 | 10.898 | 1.00 | 0.67 | 1SG | 917 |
| ATOM | 917 | OE1 | GLN | 114 | 4.180 | 1.681 | 11.184 | 1.00 | 0.67 | 1SG | 918 |
| ATOM | 918 | NE2 | GLN | 114 | 2.361 | 1.318 | 9.878 | 1.00 | 0.67 | 1SG | 919 |
| ATOM | 919 | C | GLN | 114 | −0.277 | 3.523 | 13.094 | 1.00 | 0.67 | 1SG | 920 |
| ATOM | 920 | O | GLN | 114 | 0.628 | 3.614 | 13.921 | 1.00 | 0.67 | 1SG | 921 |
| ATOM | 921 | N | ARG | 115 | −1.464 | 2.980 | 13.394 | 1.00 | 0.96 | 1SG | 922 |
| ATOM | 922 | CA | ARG | 115 | −1.598 | 2.414 | 14.692 | 1.00 | 0.96 | 1SG | 923 |
| ATOM | 923 | CB | ARG | 115 | −2.998 | 2.600 | 15.301 | 1.00 | 0.96 | 1SG | 924 |
| ATOM | 924 | CG | ARG | 115 | −3.211 | 4.035 | 15.805 | 1.00 | 0.96 | 1SG | 925 |
| ATOM | 925 | CD | ARG | 115 | −4.560 | 4.289 | 16.481 | 1.00 | 0.96 | 1SG | 926 |
| ATOM | 926 | NE | ARG | 115 | −5.574 | 4.464 | 15.406 | 1.00 | 0.96 | 1SG | 927 |
| ATOM | 927 | CZ | ARG | 115 | −6.562 | 5.393 | 15.549 | 1.00 | 0.96 | 1SG | 928 |
| ATOM | 928 | NH1 | ARG | 115 | −6.599 | 6.163 | 16.676 | 1.00 | 0.96 | 1SG | 929 |
| ATOM | 929 | NH2 | ARG | 115 | −7.494 | 5.545 | 14.567 | 1.00 | 0.96 | 1SG | 930 |
| ATOM | 930 | C | ARG | 115 | −1.244 | 0.977 | 14.546 | 1.00 | 0.96 | 1SG | 931 |
| ATOM | 931 | O | ARG | 115 | −1.391 | 0.414 | 13.462 | 1.00 | 0.96 | 1SG | 932 |
| ATOM | 932 | N | HIS | 116 | −0.722 | 0.342 | 15.612 | 1.00 | 0.72 | 1SG | 933 |
| ATOM | 933 | CA | HIS | 116 | −0.366 | −1.017 | 15.373 | 1.00 | 0.72 | 1SG | 934 |
| ATOM | 934 | ND1 | HIS | 116 | 2.266 | −2.409 | 14.267 | 1.00 | 0.72 | 1SG | 935 |
| ATOM | 935 | CG | HIS | 116 | 1.472 | −2.682 | 15.361 | 1.00 | 0.72 | 1SG | 936 |
| ATOM | 936 | CB | HIS | 116 | 0.806 | −1.598 | 16.177 | 1.00 | 0.72 | 1SG | 937 |
| ATOM | 937 | NE2 | HIS | 116 | 2.191 | −4.622 | 14.448 | 1.00 | 0.72 | 1SG | 938 |
| ATOM | 938 | CD2 | HIS | 116 | 1.438 | −4.041 | 15.458 | 1.00 | 0.72 | 1SG | 939 |
| ATOM | 939 | CE1 | HIS | 116 | 2.669 | −3.603 | 13.759 | 1.00 | 0.72 | 1SG | 940 |
| ATOM | 940 | C | HIS | 116 | −1.576 | −1.890 | 15.500 | 1.00 | 0.72 | 1SG | 941 |
| ATOM | 941 | O | HIS | 116 | −2.709 | −1.421 | 15.592 | 1.00 | 0.72 | 1SG | 942 |
| ATOM | 942 | N | GLY | 117 | −1.355 | −3.212 | 15.500 | 1.00 | 0.51 | 1SG | 943 |
| ATOM | 943 | CA | GLY | 117 | −2.431 | −4.157 | 15.415 | 1.00 | 0.51 | 1SG | 944 |
| ATOM | 944 | C | GLY | 117 | −3.410 | −4.063 | 16.545 | 1.00 | 0.51 | 1SG | 945 |
| ATOM | 945 | O | GLY | 117 | −4.616 | −4.166 | 16.341 | 1.00 | 0.51 | 1SG | 946 |
| ATOM | 946 | N | ASP | 118 | −2.928 | −3.884 | 17.778 | 1.00 | 0.60 | 1SG | 947 |
| ATOM | 947 | CA | ASP | 118 | −3.770 | −3.962 | 18.940 | 1.00 | 0.60 | 1SG | 948 |
| ATOM | 948 | CB | ASP | 118 | −2.936 | −3.901 | 20.206 | 1.00 | 0.60 | 1SG | 949 |
| ATOM | 949 | CG | ASP | 118 | −2.013 | −5.059 | 19.984 | 1.00 | 0.60 | 1SG | 950 |
| ATOM | 950 | OD1 | ASP | 118 | −2.390 | −6.205 | 20.352 | 1.00 | 0.60 | 1SG | 951 |
| ATOM | 951 | OD2 | ASP | 118 | −0.897 | −4.824 | 19.446 | 1.00 | 0.60 | 1SG | 952 |
| ATOM | 952 | C | ASP | 118 | −4.720 | −2.826 | 19.010 | 1.00 | 0.60 | 1SG | 953 |
| ATOM | 953 | O | ASP | 118 | −5.817 | −2.932 | 19.557 | 1.00 | 0.60 | 1SG | 954 |
| ATOM | 954 | N | ILE | 119 | −4.287 | −1.689 | 18.473 | 1.00 | 0.76 | 1SG | 955 |
| ATOM | 955 | CA | ILE | 119 | −4.986 | −0.466 | 18.666 | 1.00 | 0.76 | 1SG | 956 |
| ATOM | 956 | CB | ILE | 119 | −4.404 | 0.581 | 17.768 | 1.00 | 0.76 | 1SG | 957 |
| ATOM | 957 | CG2 | ILE | 119 | −5.344 | 1.791 | 17.822 | 1.00 | 0.76 | 1SG | 958 |
| ATOM | 958 | CG1 | ILE | 119 | −2.947 | 0.917 | 18.132 | 1.00 | 0.76 | 1SG | 959 |
| ATOM | 959 | CG1 | ILE | 119 | −1.933 | −0.222 | 18.067 | 1.00 | 0.76 | 1SG | 960 |
| ATOM | 960 | C | ILE | 119 | −6.420 | −0.576 | 18.266 | 1.00 | 0.76 | 1SG | 961 |
| ATOM | 961 | O | ILE | 119 | −7.323 | −0.598 | 19.101 | 1.00 | 0.76 | 1SG | 962 |
| ATOM | 962 | N | ILE | 120 | −6.645 | −0.651 | 16.946 | 1.00 | 0.92 | 1SG | 963 |
| ATOM | 963 | CA | ILE | 120 | −7.960 | −0.632 | 16.391 | 1.00 | 0.92 | 1SG | 964 |
| ATOM | 964 | CB | ILE | 120 | −7.984 | −0.066 | 14.993 | 1.00 | 0.92 | 1SG | 965 |
| ATOM | 965 | CG2 | ILE | 120 | −7.579 | 1.416 | 15.070 | 1.00 | 0.92 | 1SG | 966 |
| ATOM | 966 | CG1 | ILE | 120 | −7.112 | −0.893 | 14.024 | 1.00 | 0.92 | 1SG | 967 |
| ATOM | 967 | CD1 | ILE | 120 | −5.609 | −0.868 | 14.325 | 1.00 | 0.92 | 1SG | 968 |
| ATOM | 968 | C | ILE | 120 | −8.595 | −1.978 | 16.351 | 1.00 | 0.92 | 1SG | 969 |
| ATOM | 969 | O | ILE | 120 | −9.814 | −2.059 | 16.489 | 1.00 | 0.92 | 1SG | 970 |
| ATOM | 970 | N | GLN | 121 | −7.776 | −3.039 | 16.162 | 1.00 | 0.88 | 1SG | 971 |
| ATOM | 971 | CA | GLN | 121 | −8.223 | −4.376 | 15.864 | 1.00 | 0.88 | 1SG | 972 |
| ATOM | 972 | CB | GLN | 121 | −7.062 | −5.309 | 15.460 | 1.00 | 0.88 | 1SG | 973 |
| ATOM | 973 | CG | GLN | 121 | −6.251 | −4.817 | 14.250 | 1.00 | 0.88 | 1SG | 974 |
| ATOM | 974 | CD | GLN | 121 | −6.750 | −5.449 | 12.960 | 1.00 | 0.88 | 1SG | 975 |
| ATOM | 975 | OE1 | GLN | 121 | −7.762 | −6.146 | 12.935 | 1.00 | 0.88 | 1SG | 976 |
| ATOM | 976 | NE2 | GLN | 121 | −6.000 | −5.216 | 11.847 | 1.00 | 0.88 | 1SG | 977 |
| ATOM | 977 | C | GLN | 121 | −8.928 | −5.020 | 17.016 | 1.00 | 0.88 | 1SG | 978 |
| ATOM | 978 | O | GLN | 121 | −8.476 | −6.052 | 17.508 | 1.00 | 0.88 | 1SG | 979 |
| ATOM | 979 | N | LYS | 122 | −10.086 | −4.452 | 17.416 | 1.00 | 0.77 | 1SG | 980 |
| ATOM | 980 | CA | LYS | 122 | −10.982 | −4.935 | 18.424 | 1.00 | 0.77 | 1SG | 981 |
| ATOM | 981 | CB | LYS | 122 | −11.401 | −6.407 | 18.217 | 1.00 | 0.77 | 1SG | 982 |
| ATOM | 982 | CG | LYS | 122 | −12.186 | −6.671 | 16.930 | 1.00 | 0.77 | 1SG | 983 |
| ATOM | 983 | CD | LYS | 122 | −12.385 | −8.163 | 16.635 | 1.00 | 0.77 | 1SG | 984 |
| ATOM | 984 | CE | LYS | 122 | −11.082 | −8.970 | 16.584 | 1.00 | 0.77 | 1SG | 985 |
| ATOM | 985 | NZ | LYS | 122 | −10.141 | −8.364 | 15.618 | 1.00 | 0.77 | 1SG | 986 |
| ATOM | 986 | C | LYS | 122 | −10.368 | −4.843 | 19.783 | 1.00 | 0.77 | 1SG | 987 |
| ATOM | 987 | O | LYS | 122 | −11.013 | −4.405 | 20.736 | 1.00 | 0.77 | 1SG | 988 |
| ATOM | 988 | N | ASP | 123 | −9.091 | −5.238 | 19.899 | 1.00 | 0.74 | 1SG | 989 |
| ATOM | 989 | CA | ASP | 123 | −8.471 | −5.416 | 21.174 | 1.00 | 0.74 | 1SG | 990 |
| ATOM | 990 | CB | ASP | 123 | −7.031 | −5.962 | 21.030 | 1.00 | 0.74 | 1SG | 991 |
| ATOM | 991 | CG | ASP | 123 | −6.490 | −6.386 | 22.387 | 1.00 | 0.74 | 1SG | 992 |
| ATOM | 992 | OD1 | ASP | 123 | −7.301 | −6.486 | 23.347 | 1.00 | 0.74 | 1SG | 993 |
| ATOM | 993 | OD2 | ASP | 123 | −5.257 | −6.628 | 22.481 | 1.00 | 0.74 | 1SG | 994 |
| ATOM | 994 | C | ASP | 123 | −8.397 | −4.158 | 21.983 | 1.00 | 0.74 | 1SG | 995 |
| ATOM | 995 | O | ASP | 123 | −9.004 | −4.076 | 23.050 | 1.00 | 0.74 | 1SG | 996 |
| ATOM | 996 | N | PHE | 124 | −7.678 | −3.138 | 21.478 | 1.00 | 0.63 | 1SG | 997 |
| ATOM | 997 | CA | PHE | 124 | −7.363 | −1.984 | 22.275 | 1.00 | 0.63 | 1SG | 998 |
| ATOM | 998 | CB | PHE | 124 | −6.296 | −1.103 | 21.592 | 1.00 | 0.63 | 1SG | 999 |
| ATOM | 999 | CG | PHE | 124 | −5.743 | −0.166 | 22.608 | 1.00 | 0.63 | 1SG | 1000 |
| ATOM | 1000 | CD1 | PHE | 124 | −6.311 | 1.065 | 22.824 | 1.00 | 0.63 | 1SG | 1001 |
| ATOM | 1001 | CD2 | PHE | 124 | −4.645 | −0.540 | 23.351 | 1.00 | 0.63 | 1SG | 1002 |
| ATOM | 1002 | CE1 | PHE | 124 | −5.781 | 1.907 | 23.774 | 1.00 | 0.63 | 1SG | 1003 |
| ATOM | 1003 | CE2 | PHE | 124 | −4.110 | 0.296 | 24.303 | 1.00 | 0.63 | 1SG | 1004 |
| ATOM | 1004 | CZ | PHE | 124 | −4.681 | 1.526 | 24.509 | 1.00 | 0.63 | 1SG | 1005 |
| ATOM | 1005 | C | PHE | 124 | −8.551 | −1.118 | 22.573 | 1.00 | 0.63 | 1SG | 1006 |
| ATOM | 1006 | O | PHE | 124 | −8.862 | −0.851 | 23.732 | 1.00 | 0.63 | 1SG | 1007 |
| ATOM | 1007 | N | LEU | 125 | −9.258 | −0.676 | 21.523 | 1.00 | 0.59 | 1SG | 1008 |
| ATOM | 1008 | CA | LEU | 125 | −10.269 | 0.330 | 21.682 | 1.00 | 0.59 | 1SG | 1009 |
| ATOM | 1009 | CB | LEU | 125 | −10.786 | 0.844 | 20.341 | 1.00 | 0.59 | 1SG | 1010 |
| ATOM | 1010 | CG | LEU | 125 | −9.670 | 1.444 | 19.482 | 1.00 | 0.59 | 1SG | 1011 |
| ATOM | 1011 | CD2 | LEU | 125 | −8.691 | 2.272 | 20.335 | 1.00 | 0.59 | 1SG | 1012 |
| ATOM | 1012 | CD1 | LEU | 125 | −10.261 | 2.199 | 18.280 | 1.00 | 0.59 | 1SG | 1013 |
| ATOM | 1013 | C | LEU | 125 | −11.478 | −0.146 | 22.409 | 1.00 | 0.59 | 1SG | 1014 |
| ATOM | 1014 | O | LEU | 125 | −11.983 | 0.534 | 23.299 | 1.00 | 0.59 | 1SG | 1015 |
| ATOM | 1015 | N | ASP | 126 | −11.979 | −1.340 | 22.065 | 1.00 | 0.75 | 1SG | 1016 |
| ATOM | 1016 | CA | ASP | 126 | −13.281 | −1.658 | 22.574 | 1.00 | 0.75 | 1SG | 1017 |
| ATOM | 1017 | CB | ASP | 126 | −14.167 | −2.352 | 21.518 | 1.00 | 0.75 | 1SG | 1018 |
| ATOM | 1018 | CG | ASP | 126 | −15.628 | −2.254 | 21.935 | 1.00 | 0.75 | 1SG | 1019 |
| ATOM | 1019 | OD1 | ASP | 126 | −15.968 | −2.742 | 23.043 | 1.00 | 0.75 | 1SG | 1020 |
| ATOM | 1020 | OD2 | ASP | 126 | −16.429 | −1.691 | 21.138 | 1.00 | 0.75 | 1SG | 1021 |
| ATOM | 1021 | C | ASP | 126 | −13.201 | −2.543 | 23.768 | 1.00 | 0.75 | 1SG | 1022 |
| ATOM | 1022 | O | ASP | 126 | −13.082 | −3.761 | 23.649 | 1.00 | 0.75 | 1SG | 1023 |
| ATOM | 1023 | N | VAL | 127 | −13.279 | −1.945 | 24.970 | 1.00 | 0.98 | 1SG | 1024 |
| ATOM | 1024 | CA | VAL | 127 | −13.328 | −2.735 | 26.163 | 1.00 | 0.98 | 1SG | 1025 |
| ATOM | 1025 | CB | VAL | 127 | −12.041 | −3.437 | 26.498 | 1.00 | 0.98 | 1SG | 1026 |
| ATOM | 1026 | CG1 | VAL | 127 | −10.930 | −2.413 | 26.798 | 1.00 | 0.98 | 1SG | 1027 |
| ATOM | 1027 | CG2 | VAL | 127 | −12.342 | −4.357 | 27.692 | 1.00 | 0.98 | 1SG | 1028 |
| ATOM | 1028 | C | VAL | 127 | −13.653 | −1.796 | 27.283 | 1.00 | 0.98 | 1SG | 1029 |
| ATOM | 1029 | O | VAL | 127 | −14.447 | −2.103 | 28.173 | 1.00 | 0.98 | 1SG | 1030 |
| ATOM | 1030 | N | TYR | 128 | −13.042 | −0.601 | 27.225 | 1.00 | 1.02 | 1SG | 1031 |
| ATOM | 1031 | CA | TYR | 128 | −13.167 | 0.446 | 28.191 | 1.00 | 1.02 | 1SG | 1032 |
| ATOM | 1032 | CB | TYR | 128 | −11.903 | 1.306 | 28.352 | 1.00 | 1.02 | 1SG | 1033 |
| ATOM | 1033 | CG | TYR | 128 | −11.375 | 1.766 | 27.042 | 1.00 | 1.02 | 1SG | 1034 |
| ATOM | 1034 | CG1 | TYR | 128 | −10.498 | 0.941 | 26.380 | 1.00 | 1.02 | 1SG | 1035 |
| ATOM | 1035 | CD2 | TYR | 128 | −11.721 | 2.977 | 26.481 | 1.00 | 1.02 | 1SG | 1036 |
| ATOM | 1036 | CE1 | TYR | 128 | −9.964 | 1.304 | 25.174 | 1.00 | 1.02 | 1SG | 1037 |
| ATOM | 1037 | CE2 | TYR | 128 | −11.186 | 3.351 | 25.268 | 1.00 | 1.02 | 1SG | 1038 |
| ATOM | 1038 | CZ | TYR | 128 | −10.311 | 2.507 | 24.621 | 1.00 | 1.02 | 1SG | 1039 |
| ATOM | 1039 | OH | TYR | 128 | −9.749 | 2.862 | 23.382 | 1.00 | 1.02 | 1SG | 1040 |
| ATOM | 1040 | C | TYR | 128 | −14.414 | 1.245 | 28.027 | 1.00 | 1.02 | 1SG | 1041 |
| ATOM | 1041 | O | TYR | 128 | −14.707 | 2.101 | 28.861 | 1.00 | 1.02 | 1SG | 1042 |
| ATOM | 1042 | N | TYR | 129 | −15.170 | 0.974 | 26.950 | 1.00 | 0.75 | 1SG | 1043 |
| ATOM | 1043 | CA | TYR | 129 | −16.342 | 1.716 | 26.605 | 1.00 | 0.75 | 1SG | 1044 |
| ATOM | 1044 | CB | TYR | 129 | −17.160 | 0.962 | 25.539 | 1.00 | 0.75 | 1SG | 1045 |
| ATOM | 1045 | CG | TYR | 129 | −18.343 | 1.729 | 25.060 | 1.00 | 0.75 | 1SG | 1046 |
| ATOM | 1046 | CG1 | TYR | 129 | −18.221 | 2.589 | 23.988 | 1.00 | 0.75 | 1SG | 1047 |
| ATOM | 1047 | CD2 | TYR | 129 | −19.575 | 1.569 | 25.650 | 1.00 | 0.75 | 1SG | 1048 |
| ATOM | 1048 | CE1 | TYR | 129 | −19.300 | 3.291 | 23.510 | 1.00 | 0.75 | 1SG | 1049 |
| ATOM | 1049 | CE2 | TYR | 129 | −20.657 | 2.270 | 25.170 | 1.00 | 0.75 | 1SG | 1050 |
| ATOM | 1050 | CZ | TYR | 129 | −20.528 | 3.134 | 24.105 | 1.00 | 0.75 | 1SG | 1051 |
| ATOM | 1051 | OH | TYR | 129 | −21.640 | 3.851 | 23.613 | 1.00 | 0.75 | 1SG | 1052 |
| ATOM | 1052 | C | TYR | 129 | −17.213 | 1.844 | 27.811 | 1.00 | 0.75 | 1SG | 1053 |
| ATOM | 1053 | O | TYR | 129 | −17.537 | 2.961 | 28.209 | 1.00 | 0.75 | 1SG | 1054 |
| ATOM | 1054 | N | ASN | 130 | −17.582 | 0.731 | 28.469 | 1.00 | 0.49 | 1SG | 1055 |
| ATOM | 1055 | CA | ASN | 130 | −18.489 | 0.863 | 29.574 | 1.00 | 0.49 | 1SG | 1056 |
| ATOM | 1056 | CB | ASN | 130 | −18.907 | −0.498 | 30.158 | 1.00 | 0.49 | 1SG | 1057 |
| ATOM | 1057 | CG | ASN | 130 | −19.994 | −0.335 | 31.222 | 1.00 | 0.49 | 1SG | 1058 |
| ATOM | 1058 | OD1 | ASN | 130 | −19.918 | 0.485 | 32.136 | 1.00 | 0.49 | 1SG | 1059 |
| ATOM | 1059 | ND2 | ASN | 130 | −21.051 | −1.181 | 31.100 | 1.00 | 0.49 | 1SG | 1060 |
| ATOM | 1060 | C | ASN | 130 | −17.884 | 1.639 | 30.708 | 1.00 | 0.49 | 1SG | 1061 |
| ATOM | 1061 | O | ASN | 130 | −18.156 | 2.824 | 30.901 | 1.00 | 0.49 | 1SG | 1062 |
| ATOM | 1062 | N | LEU | 131 | −17.010 | 0.963 | 31.472 | 1.00 | 0.50 | 1SG | 1063 |
| ATOM | 1063 | CA | LEU | 131 | −16.576 | 1.513 | 32.723 | 1.00 | 0.50 | 1SG | 1064 |
| ATOM | 1064 | CB | LEU | 131 | −15.893 | 0.490 | 33.642 | 1.00 | 0.50 | 1SG | 1065 |
| ATOM | 1065 | CG | LEU | 131 | −15.575 | 1.080 | 35.035 | 1.00 | 0.50 | 1SG | 1066 |
| ATOM | 1066 | CD2 | LEU | 131 | −14.692 | 0.163 | 35.881 | 1.00 | 0.50 | 1SG | 1067 |
| ATOM | 1067 | CD1 | LEU | 131 | −16.857 | 1.417 | 35.799 | 1.00 | 0.50 | 1SG | 1068 |
| ATOM | 1068 | C | LEU | 131 | −15.652 | 2.676 | 32.639 | 1.00 | 0.50 | 1SG | 1069 |
| ATOM | 1069 | O | LEU | 131 | −15.862 | 3.666 | 33.340 | 1.00 | 0.50 | 1SG | 1070 |
| ATOM | 1070 | N | THR | 132 | −14.610 | 2.625 | 31.789 | 1.00 | 0.78 | 1SG | 1071 |
| ATOM | 1071 | CA | THR | 132 | −13.644 | 3.664 | 31.989 | 1.00 | 0.78 | 1SG | 1072 |
| ATOM | 1072 | CB | THR | 132 | −12.295 | 3.418 | 31.316 | 1.00 | 0.78 | 1SG | 1073 |
| ATOM | 1073 | OG1 | THR | 132 | −11.297 | 4.199 | 31.952 | 1.00 | 0.78 | 1SG | 1074 |
| ATOM | 1074 | CG2 | THR | 132 | −12.292 | 3.799 | 29.829 | 1.00 | 0.78 | 1SG | 1075 |
| ATOM | 1075 | C | THR | 132 | −14.201 | 5.013 | 31.643 | 1.00 | 0.78 | 1SG | 1076 |
| ATOM | 1076 | O | THR | 132 | −14.313 | 5.872 | 32.517 | 1.00 | 0.78 | 1SG | 1077 |
| ATOM | 1077 | N | LEU | 133 | −14.613 | 5.230 | 30.381 | 1.00 | 1.02 | 1SG | 1078 |
| ATOM | 1078 | CA | LEU | 133 | −14.951 | 6.570 | 30.019 | 1.00 | 1.02 | 1SG | 1079 |
| ATOM | 1079 | CB | LEU | 133 | −15.097 | 6.783 | 28.499 | 1.00 | 1.02 | 1SG | 1080 |
| ATOM | 1080 | CG | LEU | 133 | −13.888 | 6.331 | 27.650 | 1.00 | 1.02 | 1SG | 1081 |
| ATOM | 1081 | CD2 | LEU | 133 | −13.825 | 4.798 | 27.514 | 1.00 | 1.02 | 1SG | 1082 |
| ATOM | 1082 | CD1 | LEU | 133 | −12.576 | 6.968 | 28.141 | 1.00 | 1.02 | 1SG | 1083 |
| ATOM | 1083 | C | LEU | 133 | −16.243 | 7.059 | 30.601 | 1.00 | 1.02 | 1SG | 1084 |
| ATOM | 1084 | O | LEU | 133 | −16.280 | 8.070 | 31.300 | 1.00 | 1.02 | 1SG | 1085 |
| ATOM | 1085 | N | LYS | 134 | −17.344 | 6.339 | 30.310 | 1.00 | 0.93 | 1SG | 1086 |
| ATOM | 1086 | CA | LYS | 134 | −18.641 | 6.883 | 30.588 | 1.00 | 0.93 | 1SG | 1087 |
| ATOM | 1087 | CB | LYS | 134 | −19.772 | 6.199 | 29.793 | 1.00 | 0.93 | 1SG | 1088 |
| ATOM | 1088 | CG | LYS | 134 | −20.239 | 4.839 | 30.311 | 1.00 | 0.93 | 1SG | 1089 |
| ATOM | 1089 | CD | LYS | 134 | −21.213 | 4.954 | 31.481 | 1.00 | 0.93 | 1SG | 1090 |
| ATOM | 1090 | CE | LYS | 134 | −21.744 | 3.612 | 31.973 | 1.00 | 0.93 | 1SG | 1091 |
| ATOM | 1091 | NZ | LYS | 134 | −22.938 | 3.826 | 32.822 | 1.00 | 0.93 | 1SG | 1092 |
| ATOM | 1092 | C | LYS | 134 | −18.997 | 6.945 | 32.038 | 1.00 | 0.93 | 1SG | 1093 |
| ATOM | 1093 | O | LYS | 134 | −19.405 | 8.003 | 32.514 | 1.00 | 0.93 | 1SG | 1094 |
| ATOM | 1094 | N | THR | 135 | −18.857 | 5.840 | 32.794 | 1.00 | 0.72 | 1SG | 1095 |
| ATOM | 1095 | CA | THR | 135 | −19.305 | 5.929 | 34.157 | 1.00 | 0.72 | 1SG | 1096 |
| ATOM | 1096 | CB | THR | 135 | −19.418 | 4.618 | 34.876 | 1.00 | 0.72 | 1SG | 1097 |
| ATOM | 1097 | OG1 | THR | 135 | −20.026 | 4.815 | 36.147 | 1.00 | 0.72 | 1SG | 1098 |
| ATOM | 1098 | CG2 | THR | 135 | −18.019 | 4.002 | 35.023 | 1.00 | 0.72 | 1SG | 1099 |
| ATOM | 1099 | C | THR | 135 | −18.397 | 6.800 | 34.949 | 1.00 | 0.72 | 1SG | 1100 |
| ATOM | 1100 | O | THR | 135 | −18.849 | 7.626 | 35.739 | 1.00 | 0.72 | 1SG | 1101 |
| ATOM | 1101 | N | MET | 136 | −17.078 | 6.662 | 34.747 | 1.00 | 0.61 | 1SG | 1102 |
| ATOM | 1102 | CA | MET | 136 | −16.215 | 7.441 | 35.576 | 1.00 | 0.61 | 1SG | 1103 |
| ATOM | 1103 | CB | MET | 136 | −14.854 | 6.763 | 35.796 | 1.00 | 0.61 | 1SG | 1104 |
| ATOM | 1104 | CG | MET | 136 | −14.268 | 7.003 | 37.190 | 1.00 | 0.61 | 1SG | 1105 |
| ATOM | 1105 | SD | MET | 136 | −13.821 | 8.715 | 37.594 | 1.00 | 0.61 | 1SG | 1106 |
| ATOM | 1106 | CE | MET | 136 | −12.176 | 8.604 | 36.849 | 1.00 | 0.61 | 1SG | 1107 |
| ATOM | 1107 | C | MET | 136 | −16.016 | 8.739 | 34.863 | 1.00 | 0.61 | 1SG | 1108 |
| ATOM | 1108 | O | MET | 136 | −15.390 | 8.799 | 33.807 | 1.00 | 0.61 | 1SG | 1109 |
| ATOM | 1109 | N | MET | 137 | −16.582 | 9.821 | 35.422 | 1.00 | 0.44 | 1SG | 1110 |
| ATOM | 1110 | CA | MET | 137 | −16.475 | 11.121 | 34.827 | 1.00 | 0.44 | 1SG | 1111 |
| ATOM | 1111 | CB | MET | 137 | −17.288 | 12.195 | 35.569 | 1.00 | 0.44 | 1SG | 1112 |
| ATOM | 1112 | CG | MET | 137 | −16.681 | 12.621 | 36.907 | 1.00 | 0.44 | 1SG | 1113 |
| ATOM | 1113 | SD | MET | 137 | −17.562 | 13.997 | 37.699 | 1.00 | 0.44 | 1SG | 1114 |
| ATOM | 1114 | CE | MET | 137 | −16.325 | 14.240 | 39.005 | 1.00 | 0.44 | 1SG | 1115 |
| ATOM | 1115 | C | MET | 137 | −15.042 | 11.534 | 34.884 | 1.00 | 0.44 | 1SG | 1116 |
| ATOM | 1116 | O | MET | 137 | −14.532 | 12.205 | 33.988 | 1.00 | 0.44 | 1SG | 1117 |
| ATOM | 1117 | N | GLY | 138 | −14.356 | 11.108 | 35.958 | 1.00 | 0.44 | 1SG | 1118 |
| ATOM | 1118 | CA | GLY | 138 | −12.996 | 11.474 | 36.215 | 1.00 | 0.44 | 1SG | 1119 |
| ATOM | 1119 | C | GLY | 138 | −12.136 | 11.064 | 35.066 | 1.00 | 0.44 | 1SG | 1120 |
| ATOM | 1120 | O | GLY | 138 | −11.107 | 11.687 | 34.814 | 1.00 | 0.44 | 1SG | 1121 |
| ATOM | 1121 | N | ILE | 139 | −12.495 | 9.973 | 34.363 | 1.00 | 0.81 | 1SG | 1122 |
| ATOM | 1122 | CA | ILE | 139 | −11.651 | 9.560 | 33.274 | 1.00 | 0.81 | 1SG | 1123 |
| ATOM | 1123 | CB | ILE | 139 | −10.931 | 8.294 | 33.590 | 1.00 | 0.81 | 1SG | 1124 |
| ATOM | 1124 | CG2 | ILE | 139 | −10.022 | 8.610 | 34.781 | 1.00 | 0.81 | 1SG | 1125 |
| ATOM | 1125 | CG1 | ILE | 139 | −11.920 | 7.155 | 33.874 | 1.00 | 0.81 | 1SG | 1126 |
| ATOM | 1126 | CD1 | ILE | 139 | −11.208 | 5.844 | 34.183 | 1.00 | 0.81 | 1SG | 1127 |
| ATOM | 1127 | C | ILE | 139 | −12.459 | 9.407 | 32.037 | 1.00 | 0.81 | 1SG | 1128 |
| ATOM | 1128 | O | ILE | 139 | −13.561 | 8.868 | 32.062 | 1.00 | 0.81 | 1SG | 1129 |
| ATOM | 1129 | N | GLU | 140 | −11.895 | 9.908 | 30.921 | 1.00 | 1.00 | 1SG | 1130 |
| ATOM | 1130 | CA | GLU | 140 | −12.459 | 9.986 | 29.597 | 1.00 | 1.00 | 1SG | 1131 |
| ATOM | 1131 | CB | GLU | 140 | −13.977 | 9.731 | 29.512 | 1.00 | 1.00 | 1SG | 1132 |
| ATOM | 1132 | CG | GLU | 140 | −14.795 | 10.682 | 30.393 | 1.00 | 1.00 | 1SG | 1133 |
| ATOM | 1133 | CD | GLU | 140 | −16.269 | 10.570 | 30.025 | 1.00 | 1.00 | 1SG | 1134 |
| ATOM | 1134 | OE1 | GLU | 140 | −16.579 | 10.049 | 28.919 | 1.00 | 1.00 | 1SG | 1135 |
| ATOM | 1135 | OE2 | GLU | 140 | −17.109 | 11.015 | 30.851 | 1.00 | 1.00 | 1SG | 1136 |
| ATOM | 1136 | C | GLU | 140 | −12.277 | 11.430 | 29.353 | 1.00 | 1.00 | 1SG | 1137 |
| ATOM | 1137 | O | GLU | 140 | −13.110 | 12.125 | 28.776 | 1.00 | 1.00 | 1SG | 1138 |
| ATOM | 1138 | N | TRP | 141 | −11.118 | 11.901 | 29.823 | 1.00 | 0.72 | 1SG | 1139 |
| ATOM | 1139 | CA | TRP | 141 | −10.927 | 13.300 | 29.938 | 1.00 | 0.72 | 1SG | 1140 |
| ATOM | 1140 | CB | TRP | 141 | −9.664 | 13.653 | 30.738 | 1.00 | 0.72 | 1SG | 1141 |
| ATOM | 1141 | CG | TRP | 141 | −8.344 | 13.803 | 30.033 | 1.00 | 0.72 | 1SG | 1142 |
| ATOM | 1142 | CD2 | TRP | 141 | −7.888 | 15.092 | 29.603 | 1.00 | 0.72 | 1SG | 1143 |
| ATOM | 1143 | CD1 | TRP | 141 | −7.376 | 12.910 | 29.682 | 1.00 | 0.72 | 1SG | 1144 |
| ATOM | 1144 | NE1 | TRP | 141 | −6.336 | 13.567 | 29.066 | 1.00 | 0.72 | 1SG | 1145 |
| ATOM | 1145 | CE2 | TRP | 141 | −6.643 | 14.909 | 29.012 | 1.00 | 0.72 | 1SG | 1146 |
| ATOM | 1146 | CE3 | TRP | 141 | −8.465 | 16.325 | 29.701 | 1.00 | 0.72 | 1SG | 1147 |
| ATOM | 1147 | CZ2 | TRP | 141 | −5.952 | 15.968 | 28.508 | 1.00 | 0.72 | 1SG | 1148 |
| ATOM | 1148 | CZ3 | TRP | 141 | −7.764 | 17.392 | 29.186 | 1.00 | 0.72 | 1SG | 1149 |
| ATOM | 1149 | CH2 | TRP | 141 | −6.529 | 17.214 | 28.601 | 1.00 | 0.72 | 1SG | 1150 |
| ATOM | 1150 | C | TRP | 141 | −10.847 | 14.025 | 28.639 | 1.00 | 0.72 | 1SG | 1151 |
| ATOM | 1151 | O | TRP | 141 | −11.682 | 14.895 | 28.413 | 1.00 | 0.72 | 1SG | 1152 |
| ATOM | 1152 | N | VAL | 142 | −9.923 | 13.596 | 27.745 | 1.00 | 0.45 | 1SG | 1153 |
| ATOM | 1153 | CA | VAL | 142 | −9.420 | 14.282 | 26.580 | 1.00 | 0.45 | 1SG | 1154 |
| ATOM | 1154 | CB | VAL | 142 | −9.310 | 13.443 | 25.346 | 1.00 | 0.45 | 1SG | 1155 |
| ATOM | 1155 | CG1 | VAL | 142 | −8.825 | 14.374 | 24.231 | 1.00 | 0.45 | 1SG | 1156 |
| ATOM | 1156 | CG2 | VAL | 142 | −8.321 | 12.293 | 25.600 | 1.00 | 0.45 | 1SG | 1157 |
| ATOM | 1157 | C | VAL | 142 | −10.145 | 15.550 | 26.251 | 1.00 | 0.45 | 1SG | 1158 |
| ATOM | 1158 | O | VAL | 142 | −11.353 | 15.569 | 26.032 | 1.00 | 0.45 | 1SG | 1159 |
| ATOM | 1159 | N | HIS | 143 | −9.396 | 16.671 | 26.193 | 1.00 | 0.56 | 1SG | 1160 |
| ATOM | 1160 | CA | HIS | 143 | −10.006 | 17.925 | 25.862 | 1.00 | 0.56 | 1SG | 1161 |
| ATOM | 1161 | ND1 | HIS | 143 | −9.101 | 21.633 | 25.692 | 1.00 | 0.56 | 1SG | 1162 |
| ATOM | 1162 | CG | HIS | 143 | −9.705 | 20.403 | 25.566 | 1.00 | 0.56 | 1SG | 1163 |
| ATOM | 1163 | CB | HIS | 143 | −9.008 | 19.089 | 25.755 | 1.00 | 0.56 | 1SG | 1164 |
| ATOM | 1164 | NE2 | HIS | 143 | −11.226 | 22.021 | 25.170 | 1.00 | 0.56 | 1SG | 1165 |
| ATOM | 1165 | CD2 | HIS | 143 | −11.002 | 20.660 | 25.249 | 1.00 | 0.56 | 1SG | 1166 |
| ATOM | 1166 | CE1 | HIS | 143 | −10.055 | 22.565 | 25.445 | 1.00 | 0.56 | 1SG | 1167 |
| ATOM | 1167 | C | HIS | 143 | −10.609 | 17.702 | 24.522 | 1.00 | 0.56 | 1SG | 1168 |
| ATOM | 1168 | O | HIS | 143 | −11.698 | 18.179 | 24.212 | 1.00 | 0.56 | 1SG | 1169 |
| ATOM | 1169 | N | ARG | 144 | −9.903 | 16.926 | 23.689 | 1.00 | 0.77 | 1SG | 1170 |
| ATOM | 1170 | CA | ARG | 144 | −10.496 | 16.560 | 22.447 | 1.00 | 0.77 | 1SG | 1171 |
| ATOM | 1171 | CB | ARG | 144 | −9.661 | 15.547 | 21.660 | 1.00 | 0.77 | 1SG | 1172 |
| ATOM | 1172 | CG | ARG | 144 | −8.394 | 16.090 | 21.016 | 1.00 | 0.77 | 1SG | 1173 |
| ATOM | 1173 | CD | ARG | 144 | −7.325 | 15.006 | 20.933 | 1.00 | 0.77 | 1SG | 1174 |
| ATOM | 1174 | NE | ARG | 144 | −8.015 | 13.730 | 21.260 | 1.00 | 0.77 | 1SG | 1175 |
| ATOM | 1175 | CZ | ARG | 144 | −7.442 | 12.848 | 22.125 | 1.00 | 0.77 | 1SG | 1176 |
| ATOM | 1176 | NH1 | ARG | 144 | −6.197 | 13.091 | 22.619 | 1.00 | 0.77 | 1SG | 1177 |
| ATOM | 1177 | NH2 | ARG | 144 | −8.132 | 11.741 | 22.526 | 1.00 | 0.77 | 1SG | 1178 |
| ATOM | 1178 | C | ARG | 144 | −11.724 | 15.806 | 22.799 | 1.00 | 0.77 | 1SG | 1179 |
| ATOM | 1179 | O | ARG | 144 | −12.830 | 16.204 | 22.438 | 1.00 | 0.77 | 1SG | 1180 |
| ATOM | 1180 | N | PHE | 145 | −11.549 | 14.714 | 23.577 | 1.00 | 0.80 | 1SG | 1181 |
| ATOM | 1181 | CA | PHE | 145 | −12.654 | 13.845 | 23.839 | 1.00 | 0.80 | 1SG | 1182 |
| ATOM | 1132 | CB | PHE | 145 | −12.857 | 12.834 | 22.707 | 1.00 | 0.80 | 1SG | 1183 |
| ATOM | 1183 | CG | PHE | 145 | −13.298 | 13.661 | 21.548 | 1.00 | 0.80 | 1SG | 1184 |
| ATOM | 1184 | CD1 | PHE | 145 | −14.600 | 14.101 | 21.504 | 1.00 | 0.80 | 1SG | 1185 |
| ATOM | 1185 | CD2 | PHE | 145 | −12.432 | 14.032 | 20.543 | 1.00 | 0.80 | 1SG | 1186 |
| ATOM | 1136 | CE1 | PHE | 145 | −15.055 | 14.869 | 20.463 | 1.00 | 0.80 | 1SG | 1187 |
| ATOM | 1187 | CE2 | PHE | 145 | −12.882 | 14.802 | 19.494 | 1.00 | 0.80 | 1SG | 1188 |
| ATOM | 1188 | CZ | PHE | 145 | −14.193 | 15.218 | 19.453 | 1.00 | 0.80 | 1SG | 1189 |
| ATOM | 1189 | C | PHE | 145 | −12.520 | 13.119 | 25.153 | 1.00 | 0.80 | 1SG | 1190 |
| ATOM | 1190 | O | PHE | 145 | −12.954 | 13.628 | 26.185 | 1.00 | 0.80 | 1SG | 1191 |
| ATOM | 1191 | N | CYS | 146 | −11.947 | 11.891 | 25.164 | 1.00 | 0.79 | 1SG | 1192 |
| ATOM | 1192 | CA | CYS | 146 | −11.925 | 11.115 | 26.373 | 1.00 | 0.79 | 1SG | 1193 |
| ATOM | 1193 | CB | CYS | 146 | −13.252 | 10.372 | 26.647 | 1.00 | 0.79 | 1SG | 1194 |
| ATOM | 1194 | SG | CYS | 146 | −14.733 | 11.416 | 26.532 | 1.00 | 0.79 | 1SG | 1195 |
| ATOM | 1195 | C | CYS | 146 | −10.746 | 10.170 | 26.326 | 1.00 | 0.79 | 1SG | 1196 |
| ATOM | 1196 | O | CYS | 146 | −9.779 | 10.474 | 27.022 | 1.00 | 0.79 | 1SG | 1197 |
| ATOM | 1197 | N | PRO | 147 | −10.682 | 9.033 | 25.649 | 1.00 | 0.79 | 1SG | 1198 |
| ATOM | 1198 | CA | PRO | 147 | −9.431 | 8.341 | 25.721 | 1.00 | 0.79 | 1SG | 1199 |
| ATOM | 1199 | CD | PRO | 147 | −11.779 | 8.091 | 25.439 | 1.00 | 0.79 | 1SG | 1200 |
| ATOM | 1200 | CB | PRO | 147 | −9.670 | 6.936 | 25.165 | 1.00 | 0.79 | 1SG | 1201 |
| ATOM | 1201 | CG | PRO | 147 | −11.155 | 6.688 | 25.471 | 1.00 | 0.79 | 1SG | 1202 |
| ATOM | 1202 | C | PRO | 147 | −8.368 | 9.097 | 25.002 | 1.00 | 0.79 | 1SG | 1203 |
| ATOM | 1203 | O | PRO | 147 | −8.549 | 9.446 | 23.837 | 1.00 | 0.79 | 1SG | 1204 |
| ATOM | 1204 | N | GLN | 148 | −7.235 | 9.328 | 25.686 | 1.00 | 0.64 | 1SG | 1205 |
| ATOM | 1205 | CA | GLN | 148 | −6.070 | 9.926 | 25.114 | 1.00 | 0.64 | 1SG | 1206 |
| ATOM | 1206 | CB | GLN | 148 | −5.075 | 10.428 | 26.163 | 1.00 | 0.64 | 1SG | 1207 |
| ATOM | 1207 | CG | GLN | 148 | −3.826 | 11.056 | 25.550 | 1.00 | 0.64 | 1SG | 1208 |
| ATOM | 1208 | CD | GLN | 148 | −3.004 | 11.547 | 26.725 | 1.00 | 0.64 | 1SG | 1209 |
| ATOM | 1209 | OE1 | GLN | 148 | −1.803 | 11.801 | 26.625 | 1.00 | 0.64 | 1SG | 1210 |
| ATOM | 1210 | NE2 | GLN | 148 | −3.693 | 11.668 | 27.891 | 1.00 | 0.64 | 1SG | 1211 |
| ATOM | 1211 | C | GLN | 148 | −5.418 | 8.827 | 24.355 | 1.00 | 0.64 | 1SG | 1212 |
| ATOM | 1212 | O | GLN | 148 | −4.421 | 9.028 | 23.663 | 1.00 | 0.64 | 1SG | 1213 |
| ATOM | 1213 | N | ALA | 149 | −6.025 | 7.635 | 24.492 | 1.00 | 0.43 | 1SG | 1214 |
| ATOM | 1214 | CA | ALA | 149 | −5.640 | 6.355 | 23.991 | 1.00 | 0.43 | 1SG | 1215 |
| ATOM | 1215 | CB | ALA | 149 | −6.598 | 5.838 | 22.905 | 1.00 | 0.43 | 1SG | 1216 |
| ATOM | 1216 | C | ALA | 149 | −4.252 | 6.313 | 23.439 | 1.00 | 0.43 | 1SG | 1217 |
| ATOM | 1217 | O | ALA | 149 | −3.269 | 6.239 | 24.172 | 1.00 | 0.43 | 1SG | 1218 |
| ATOM | 1218 | N | ALA | 150 | −4.162 | 6.349 | 22.106 | 1.00 | 0.39 | 1SG | 1219 |
| ATOM | 1219 | CA | ALA | 150 | −2.989 | 6.031 | 21.355 | 1.00 | 0.39 | 1SG | 1220 |
| ATOM | 1220 | CB | ALA | 150 | −3.165 | 6.391 | 19.867 | 1.00 | 0.39 | 1SG | 1221 |
| ATOM | 1221 | C | ALA | 150 | −1.726 | 6.713 | 21.791 | 1.00 | 0.39 | 1SG | 1222 |
| ATOM | 1222 | O | ALA | 150 | −0.721 | 6.030 | 21.982 | 1.00 | 0.39 | 1SG | 1223 |
| ATOM | 1223 | N | PHE | 151 | −1.707 | 8.039 | 22.010 | 1.00 | 0.47 | 1SG | 1224 |
| ATOM | 1224 | CA | PHE | 151 | −0.390 | 8.614 | 22.107 | 1.00 | 0.47 | 1SG | 1225 |
| ATOM | 1225 | CB | PHE | 151 | −0.273 | 9.943 | 21.344 | 1.00 | 0.47 | 1SG | 1226 |
| ATOM | 1226 | CG | PHE | 151 | 1.160 | 10.348 | 21.336 | 1.00 | 0.47 | 1SG | 1227 |
| ATOM | 1227 | CD1 | PHE | 151 | 1.674 | 11.130 | 22.344 | 1.00 | 0.47 | 1SG | 1228 |
| ATOM | 1228 | CD2 | PHE | 151 | 1.992 | 9.940 | 20.319 | 1.00 | 0.47 | 1SG | 1229 |
| ATOM | 1229 | CE1 | PHE | 151 | 2.996 | 11.504 | 22.334 | 1.00 | 0.47 | 1SG | 1230 |
| ATOM | 1230 | CE2 | PHE | 151 | 3.316 | 10.311 | 20.305 | 1.00 | 0.47 | 1SG | 1231 |
| ATOM | 1231 | CZ | PHE | 151 | 3.820 | 11.095 | 21.314 | 1.00 | 0.47 | 1SG | 1232 |
| ATOM | 1232 | C | PHE | 151 | 0.101 | 8.840 | 23.499 | 1.00 | 0.47 | 1SG | 1233 |
| ATOM | 1233 | O | PHE | 151 | −0.569 | 9.416 | 24.352 | 1.00 | 0.47 | 1SG | 1234 |
| ATOM | 1234 | N | VAL | 152 | 1.348 | 8.380 | 23.724 | 1.00 | 0.55 | 1SG | 1235 |
| ATOM | 1235 | CA | VAL | 152 | 2.084 | 8.510 | 24.944 | 1.00 | 0.55 | 1SG | 1236 |
| ATOM | 1236 | CB | VAL | 152 | 2.594 | 7.185 | 25.361 | 1.00 | 0.55 | 1SG | 1237 |
| ATOM | 1237 | CG1 | VAL | 152 | 1.427 | 6.413 | 25.990 | 1.00 | 0.55 | 1SG | 1238 |
| ATOM | 1238 | CG2 | VAL | 152 | 3.067 | 6.505 | 24.062 | 1.00 | 0.55 | 1SG | 1239 |
| ATOM | 1239 | C | VAL | 152 | 3.235 | 9.425 | 24.668 | 1.00 | 0.55 | 1SG | 1240 |
| ATOM | 1240 | O | VAL | 152 | 4.165 | 9.079 | 23.942 | 1.00 | 0.55 | 1SG | 1241 |
| ATOM | 1241 | N | MET | 153 | 3.208 | 10.625 | 25.277 | 1.00 | 0.58 | 1SG | 1242 |
| ATOM | 1242 | CA | MET | 153 | 4.219 | 11.608 | 25.015 | 1.00 | 0.58 | 1SG | 1243 |
| ATOM | 1243 | CB | MET | 153 | 4.058 | 12.884 | 25.843 | 1.00 | 0.58 | 1SG | 1244 |
| ATOM | 1244 | CG | MET | 153 | 5.169 | 13.901 | 25.566 | 1.00 | 0.58 | 1SG | 1245 |
| ATOM | 1245 | SD | MET | 153 | 5.344 | 15.231 | 26.796 | 1.00 | 0.58 | 1SG | 1246 |
| ATOM | 1246 | CE | MET | 153 | 6.827 | 15.935 | 26.020 | 1.00 | 0.58 | 1SG | 1247 |
| ATOM | 1247 | C | MET | 153 | 5.555 | 11.088 | 25.410 | 1.00 | 0.58 | 1SG | 1248 |
| ATOM | 1248 | O | MET | 153 | 6.538 | 11.270 | 24.695 | 1.00 | 0.58 | 1SG | 1249 |
| ATOM | 1249 | N | LYS | 154 | 5.602 | 10.459 | 26.594 | 1.00 | 0.53 | 1SG | 1250 |
| ATOM | 1250 | CA | LYS | 154 | 6.795 | 9.977 | 27.217 | 1.00 | 0.53 | 1SG | 1251 |
| ATOM | 1251 | CB | LYS | 154 | 6.564 | 9.603 | 28.688 | 1.00 | 0.53 | 1SG | 1252 |
| ATOM | 1252 | CG | LYS | 154 | 6.116 | 10.787 | 29.545 | 1.00 | 0.53 | 1SG | 1253 |
| ATOM | 1253 | CD | LYS | 154 | 5.541 | 10.368 | 30.899 | 1.00 | 0.53 | 1SG | 1254 |
| ATOM | 1254 | CE | LYS | 154 | 5.007 | 11.534 | 31.733 | 1.00 | 0.53 | 1SG | 1255 |
| ATOM | 1255 | NZ | LYS | 154 | 4.388 | 11.018 | 32.975 | 1.00 | 0.53 | 1SG | 1256 |
| ATOM | 1256 | C | LYS | 154 | 7.325 | 8.759 | 26.535 | 1.00 | 0.53 | 1SG | 1257 |
| ATOM | 1257 | O | LYS | 154 | 8.524 | 8.510 | 26.613 | 1.00 | 0.53 | 1SG | 1258 |
| ATOM | 1258 | N | THR | 155 | 6.463 | 7.966 | 25.863 | 1.00 | 0.56 | 1SG | 1259 |
| ATOM | 1259 | CA | THR | 155 | 6.883 | 6.671 | 25.396 | 1.00 | 0.56 | 1SG | 1260 |
| ATOM | 1260 | CB | THR | 155 | 5.763 | 5.794 | 24.956 | 1.00 | 0.56 | 1SG | 1261 |
| ATOM | 1261 | OG1 | THR | 155 | 4.836 | 5.660 | 26.022 | 1.00 | 0.56 | 1SG | 1262 |
| ATOM | 1262 | CG2 | THR | 155 | 6.336 | 4.413 | 24.591 | 1.00 | 0.56 | 1SG | 1263 |
| ATOM | 1263 | C | THR | 155 | 7.921 | 6.693 | 24.312 | 1.00 | 0.56 | 1SG | 1264 |
| ATOM | 1264 | O | THR | 155 | 7.626 | 6.506 | 23.133 | 1.00 | 0.56 | 1SG | 1265 |
| ATOM | 1265 | N | ASP | 156 | 9.196 | 6.881 | 24.698 | 1.00 | 0.64 | 1SG | 1266 |
| ATOM | 1266 | CA | ASP | 156 | 10.283 | 6.780 | 23.775 | 1.00 | 0.64 | 1SG | 1267 |
| ATOM | 1267 | CB | ASP | 156 | 11.463 | 7.722 | 24.077 | 1.00 | 0.64 | 1SG | 1268 |
| ATOM | 1268 | CG | ASP | 156 | 11.027 | 9.141 | 23.745 | 1.00 | 0.64 | 1SG | 1269 |
| ATOM | 1269 | OD1 | ASP | 156 | 9.919 | 9.298 | 23.166 | 1.00 | 0.64 | 1SG | 1270 |
| ATOM | 1270 | OD2 | ASP | 156 | 11.795 | 10.087 | 24.065 | 1.00 | 0.64 | 1SG | 1271 |
| ATOM | 1271 | C | ASP | 156 | 10.774 | 5.382 | 23.941 | 1.00 | 0.64 | 1SG | 1272 |
| ATOM | 1272 | O | ASP | 156 | 10.301 | 4.655 | 24.810 | 1.00 | 0.64 | 1SG | 1273 |
| ATOM | 1273 | N | SER | 157 | 11.741 | 4.960 | 23.111 | 1.00 | 0.55 | 1SG | 1274 |
| ATOM | 1274 | CA | SER | 157 | 12.191 | 3.603 | 23.192 | 1.00 | 0.55 | 1SG | 1275 |
| ATOM | 1275 | CB | SER | 157 | 13.299 | 3.293 | 22.172 | 1.00 | 0.55 | 1SG | 1276 |
| ATOM | 1276 | OG | SER | 157 | 14.459 | 4.061 | 22.461 | 1.00 | 0.55 | 1SG | 1277 |
| ATOM | 1277 | C | SER | 157 | 12.763 | 3.345 | 24.552 | 1.00 | 0.55 | 1SG | 1278 |
| ATOM | 1278 | O | SER | 157 | 12.425 | 2.356 | 25.199 | 1.00 | 0.55 | 1SG | 1279 |
| ATOM | 1279 | N | ASP | 158 | 13.644 | 4.244 | 25.026 | 1.00 | 0.45 | 1SG | 1280 |
| ATOM | 1280 | CA | ASP | 158 | 14.318 | 4.062 | 26.283 | 1.00 | 0.45 | 1SG | 1281 |
| ATOM | 1281 | CB | ASP | 158 | 15.432 | 5.099 | 26.504 | 1.00 | 0.45 | 1SG | 1282 |
| ATOM | 1282 | CG | ASP | 158 | 16.546 | 4.810 | 25.511 | 1.00 | 0.45 | 1SG | 1283 |
| ATOM | 1283 | OD1 | ASP | 158 | 16.871 | 3.608 | 25.320 | 1.00 | 0.45 | 1SG | 1284 |
| ATOM | 1284 | OD2 | ASP | 158 | 17.092 | 5.789 | 24.934 | 1.00 | 0.45 | 1SG | 1285 |
| ATOM | 1285 | C | ASP | 158 | 13.357 | 4.201 | 27.424 | 1.00 | 0.45 | 1SG | 1286 |
| ATOM | 1286 | O | ASP | 158 | 13.489 | 3.523 | 28.442 | 1.00 | 0.45 | 1SG | 1287 |
| ATOM | 1287 | N | MET | 159 | 12.374 | 5.106 | 27.274 | 1.00 | 0.67 | 1SG | 1288 |
| ATOM | 1288 | CA | MET | 159 | 11.434 | 5.479 | 28.295 | 1.00 | 0.67 | 1SG | 1289 |
| ATOM | 1289 | CB | MET | 159 | 10.596 | 6.705 | 27.893 | 1.00 | 0.67 | 1SG | 1290 |
| ATOM | 1290 | CG | MET | 159 | 11.427 | 7.974 | 27.685 | 1.00 | 0.67 | 1SG | 1291 |
| ATOM | 1291 | SD | MET | 159 | 12.239 | 8.600 | 29.184 | 1.00 | 0.67 | 1SG | 1292 |
| ATOM | 1292 | CE | MET | 159 | 10.700 | 9.143 | 29.978 | 1.00 | 0.67 | 1SG | 1293 |
| ATOM | 1293 | C | MET | 159 | 10.465 | 4.381 | 28.624 | 1.00 | 0.67 | 1SG | 1294 |
| ATOM | 1294 | O | MET | 159 | 9.945 | 4.346 | 29.737 | 1.00 | 0.67 | 1SG | 1295 |
| ATOM | 1295 | N | PHE | 160 | 10.152 | 3.486 | 27.668 | 1.00 | 0.82 | 1SG | 1296 |
| ATOM | 1296 | CA | PHE | 160 | 9.164 | 2.463 | 27.886 | 1.00 | 0.82 | 1SG | 1297 |
| ATOM | 1297 | CB | PHE | 160 | 8.317 | 2.217 | 26.626 | 1.00 | 0.82 | 1SG | 1298 |
| ATOM | 1298 | CG | PHE | 160 | 7.511 | 0.980 | 26.799 | 1.00 | 0.82 | 1SG | 1299 |
| ATOM | 1299 | CD1 | PHE | 160 | 6.324 | 0.998 | 27.491 | 1.00 | 0.82 | 1SG | 1300 |
| ATOM | 1300 | CD2 | PHE | 160 | 7.947 | −0.201 | 26.246 | 1.00 | 0.82 | 1SG | 1301 |
| ATOM | 1301 | CE1 | PHE | 160 | 5.590 | −0.155 | 27.631 | 1.00 | 0.82 | 1SG | 1302 |
| ATOM | 1302 | CE2 | PHE | 160 | 7.214 | −1.354 | 26.387 | 1.00 | 0.82 | 1SG | 1303 |
| ATOM | 1303 | CZ | PHE | 160 | 6.031 | −1.334 | 27.084 | 1.00 | 0.82 | 1SG | 1304 |
| ATOM | 1304 | C | PHE | 160 | 9.829 | 1.176 | 28.244 | 1.00 | 0.82 | 1SG | 1305 |
| ATOM | 1305 | O | PHE | 160 | 10.870 | 0.815 | 27.698 | 1.00 | 0.82 | 1SG | 1306 |
| ATOM | 1306 | N | ILE | 161 | 9.235 | 0.441 | 29.205 | 1.00 | 0.55 | 1SG | 1307 |
| ATOM | 1307 | CA | ILE | 161 | 9.815 | −0.820 | 29.550 | 1.00 | 0.55 | 1SG | 1308 |
| ATOM | 1308 | CB | ILE | 161 | 9.755 | −1.153 | 31.021 | 1.00 | 0.55 | 1SG | 1309 |
| ATOM | 1309 | CG2 | ILE | 161 | 8.292 | −1.183 | 31.493 | 1.00 | 0.55 | 1SG | 1310 |
| ATOM | 1310 | CG1 | ILE | 161 | 10.553 | −2.435 | 31.311 | 1.00 | 0.55 | 1SG | 1311 |
| ATOM | 1311 | CD1 | ILE | 161 | 10.801 | −2.666 | 32.802 | 1.00 | 0.55 | 1SG | 1312 |
| ATOM | 1312 | C | ILE | 161 | 9.093 | −1.851 | 28.752 | 1.00 | 0.55 | 1SG | 1313 |
| ATOM | 1313 | O | ILE | 161 | 7.902 | −2.087 | 28.938 | 1.00 | 0.55 | 1SG | 1314 |
| ATOM | 1314 | N | ASN | 162 | 9.823 | −2.479 | 27.813 | 1.00 | 0.27 | 1SG | 1315 |
| ATOM | 1315 | CA | ASN | 162 | 9.268 | −3.432 | 26.902 | 1.00 | 0.27 | 1SG | 1316 |
| ATOM | 1316 | CB | ASN | 162 | 10.283 | −3.869 | 25.833 | 1.00 | 0.27 | 1SG | 1317 |
| ATOM | 1317 | CG | ASN | 162 | 10.597 | −2.647 | 24.978 | 1.00 | 0.27 | 1SG | 1318 |
| ATOM | 1318 | OD1 | ASN | 162 | 9.694 | −1.960 | 24.503 | 1.00 | 0.27 | 1SG | 1319 |
| ATOM | 1319 | ND2 | ASN | 162 | 11.913 | −2.361 | 24.783 | 1.00 | 0.27 | 1SG | 1320 |
| ATOM | 1320 | C | ASN | 162 | 8.834 | −4.639 | 27.662 | 1.00 | 0.27 | 1SG | 1321 |
| ATOM | 1321 | O | ASN | 162 | 7.813 | −5.244 | 27.339 | 1.00 | 0.27 | 1SG | 1322 |
| ATOM | 1322 | N | VAL | 163 | 9.593 | −5.005 | 28.712 | 1.00 | 0.31 | 1SG | 1323 |
| ATOM | 1323 | CA | VAL | 163 | 9.274 | −6.187 | 29.452 | 1.00 | 0.31 | 1SG | 1324 |
| ATOM | 1324 | CB | VAL | 163 | 10.162 | −6.383 | 30.645 | 1.00 | 0.31 | 1SG | 1325 |
| ATOM | 1325 | CG1 | VAL | 163 | 9.711 | −7.652 | 31.387 | 1.00 | 0.31 | 1SG | 1326 |
| ATOM | 1326 | CG2 | VAL | 163 | 11.624 | −6.422 | 30.171 | 1.00 | 0.31 | 1SG | 1327 |
| ATOM | 1327 | C | VAL | 163 | 7.888 | −6.025 | 29.966 | 1.00 | 0.31 | 1SG | 1328 |
| ATOM | 1328 | O | VAL | 163 | 7.054 | −6.919 | 29.829 | 1.00 | 0.31 | 1SG | 1329 |
| ATOM | 1329 | N | ASP | 164 | 7.596 | −4.854 | 30.553 | 1.00 | 0.58 | 1SG | 1330 |
| ATOM | 1330 | CA | ASP | 164 | 6.273 | −4.643 | 31.043 | 1.00 | 0.58 | 1SG | 1331 |
| ATOM | 1331 | CB | ASP | 164 | 6.172 | −3.467 | 32.030 | 1.00 | 0.58 | 1SG | 1332 |
| ATOM | 1332 | CG | ASP | 164 | 6.952 | −3.854 | 33.280 | 1.00 | 0.58 | 1SG | 1333 |
| ATOM | 1333 | OD1 | ASP | 164 | 6.870 | −5.046 | 33.688 | 1.00 | 0.58 | 1SG | 1334 |
| ATOM | 1334 | OD2 | ASP | 164 | 7.657 | −2.966 | 33.835 | 1.00 | 0.58 | 1SG | 1335 |
| ATOM | 1335 | C | ASP | 164 | 5.453 | −4.328 | 29.846 | 1.00 | 0.58 | 1SG | 1336 |
| ATOM | 1336 | O | ASP | 164 | 5.772 | −3.433 | 29.070 | 1.00 | 0.58 | 1SG | 1337 |
| ATOM | 1337 | N | TYR | 165 | 4.372 | −5.083 | 29.634 | 1.00 | 0.80 | 1SG | 1338 |
| ATOM | 1338 | CA | TYR | 165 | 3.587 | −4.788 | 28.484 | 1.00 | 0.80 | 1SG | 1339 |
| ATOM | 1339 | CB | TYR | 165 | 2.633 | −5.939 | 28.141 | 1.00 | 0.80 | 1SG | 1340 |
| ATOM | 1340 | CG | TYR | 165 | 3.471 | −7.165 | 28.209 | 1.00 | 0.80 | 1SG | 1341 |
| ATOM | 1341 | CD1 | TYR | 165 | 4.257 | −7.568 | 27.154 | 1.00 | 0.80 | 1SG | 1342 |
| ATOM | 1342 | CD2 | TYR | 165 | 3.467 | −7.907 | 29.367 | 1.00 | 0.80 | 1SG | 1343 |
| ATOM | 1343 | CE1 | TYR | 165 | 5.022 | −8.706 | 27.263 | 1.00 | 0.80 | 1SG | 1344 |
| ATOM | 1344 | CE2 | TYR | 165 | 4.230 | −9.044 | 29.478 | 1.00 | 0.80 | 1SG | 1345 |
| ATOM | 1345 | CZ | TYR | 165 | 5.012 | −9.445 | 28.423 | 1.00 | 0.80 | 1SG | 1346 |
| ATOM | 1346 | OH | TYR | 165 | 5.801 | −10.611 | 28.528 | 1.00 | 0.80 | 1SG | 1347 |
| ATOM | 1347 | C | TYR | 165 | 2.762 | −3.644 | 28.934 | 1.00 | 0.80 | 1SG | 1348 |
| ATOM | 1348 | O | TYR | 165 | 1.588 | −3.805 | 29.240 | 1.00 | 0.80 | 1SG | 1349 |
| ATOM | 1349 | N | LEU | 166 | 3.353 | −2.440 | 28.987 | 1.00 | 0.79 | 1SG | 1350 |
| ATOM | 1350 | CA | LEU | 166 | 2.596 | −1.331 | 29.474 | 1.00 | 0.79 | 1SG | 1351 |
| ATOM | 1351 | CB | LEU | 166 | 3.387 | −0.019 | 29.579 | 1.00 | 0.79 | 1SG | 1352 |
| ATOM | 1352 | CG | LEU | 166 | 4.439 | −0.036 | 30.704 | 1.00 | 0.79 | 1SG | 1353 |
| ATOM | 1353 | CD2 | LEU | 166 | 4.977 | 1.370 | 30.998 | 1.00 | 0.79 | 1SG | 1354 |
| ATOM | 1354 | CD1 | LEU | 166 | 5.548 | −1.060 | 30.426 | 1.00 | 0.79 | 1SG | 1355 |
| ATOM | 1355 | C | LEU | 166 | 1.425 | −1.153 | 28.568 | 1.00 | 0.79 | 1SG | 1356 |
| ATOM | 1356 | O | LEU | 166 | 0.443 | −0.519 | 28.940 | 1.00 | 0.79 | 1SG | 1357 |
| ATOM | 1357 | N | THR | 167 | 1.511 | −1.695 | 27.339 | 1.00 | 0.72 | 1SG | 1358 |
| ATOM | 1358 | CA | THR | 167 | 0.390 | −1.651 | 26.452 | 1.00 | 0.72 | 1SG | 1359 |
| ATOM | 1359 | CB | THR | 167 | 0.613 | −2.472 | 25.215 | 1.00 | 0.72 | 1SG | 1360 |
| ATOM | 1360 | OG1 | THR | 167 | 0.806 | −3.841 | 25.548 | 1.00 | 0.72 | 1SG | 1361 |
| ATOM | 1361 | CG2 | THR | 167 | 1.853 | −1.922 | 24.487 | 1.00 | 0.72 | 1SG | 1362 |
| ATOM | 1362 | C | THR | 167 | −0.755 | −2.244 | 27.210 | 1.00 | 0.72 | 1SG | 1363 |
| ATOM | 1363 | O | THR | 167 | −1.824 | −1.643 | 27.302 | 1.00 | 0.72 | 1SG | 1364 |
| ATOM | 1364 | N | GLU | 168 | −0.553 | −3.440 | 27.796 | 1.00 | 0.78 | 1SG | 1365 |
| ATOM | 1365 | CA | GLU | 168 | −1.586 | −4.001 | 28.613 | 1.00 | 0.78 | 1SG | 1366 |
| ATOM | 1366 | CB | GLU | 168 | −1.340 | −5.453 | 29.084 | 1.00 | 0.78 | 1SG | 1367 |
| ATOM | 1367 | CG | GLU | 168 | −0.256 | −5.607 | 30.156 | 1.00 | 0.78 | 1SG | 1368 |
| ATOM | 1368 | CD | GLU | 168 | −0.135 | −7.074 | 30.549 | 1.00 | 0.78 | 1SG | 1369 |
| ATOM | 1369 | OE1 | GLU | 168 | −0.832 | −7.927 | 29.936 | 1.00 | 0.78 | 1SG | 1370 |
| ATOM | 1370 | OE2 | GLU | 168 | 0.668 | −7.361 | 31.476 | 1.00 | 0.78 | 1SG | 1371 |
| ATOM | 1371 | C | GLU | 168 | −1.676 | −3.164 | 29.848 | 1.00 | 0.78 | 1SG | 1372 |
| ATOM | 1372 | O | GLU | 168 | −2.766 | −2.838 | 30.313 | 1.00 | 0.78 | 1SG | 1373 |
| ATOM | 1373 | N | LEU | 169 | −0.511 | −2.770 | 30.404 | 1.00 | 0.74 | 1SG | 1374 |
| ATOM | 1374 | CA | LEU | 169 | −0.505 | −2.054 | 31.647 | 1.00 | 0.74 | 1SG | 1375 |
| ATOM | 1375 | CB | LEU | 169 | 0.816 | −2.164 | 32.426 | 1.00 | 0.74 | 1SG | 1376 |
| ATOM | 1376 | CG | LEU | 169 | 1.097 | −3.575 | 32.967 | 1.00 | 0.74 | 1SG | 1377 |
| ATOM | 1377 | CD2 | LEU | 169 | −0.111 | −4.118 | 33.743 | 1.00 | 0.74 | 1SG | 1378 |
| ATOM | 1378 | CD1 | LEU | 169 | 2.395 | −3.612 | 33.788 | 1.00 | 0.74 | 1SG | 1379 |
| ATOM | 1379 | C | LEU | 169 | −0.724 | −0.606 | 31.404 | 1.00 | 0.74 | 1SG | 1380 |
| ATOM | 1380 | O | LEU | 169 | 0.231 | 0.170 | 31.368 | 1.00 | 0.74 | 1SG | 1381 |
| ATOM | 1381 | N | LEU | 170 | −1.989 | −0.191 | 31.232 | 1.00 | 0.52 | 1SG | 1382 |
| ATOM | 1382 | CA | LEU | 170 | −2.207 | 1.211 | 31.066 | 1.00 | 0.52 | 1SG | 1383 |
| ATOM | 1383 | CB | LEU | 170 | −3.682 | 1.589 | 30.860 | 1.00 | 0.52 | 1SG | 1384 |
| ATOM | 1384 | CG | LEU | 170 | −4.289 | 0.981 | 29.584 | 1.00 | 0.52 | 1SG | 1385 |
| ATOM | 1385 | CD2 | LEU | 170 | −3.428 | 1.292 | 28.348 | 1.00 | 0.52 | 1SG | 1386 |
| ATOM | 1386 | CD1 | LEU | 170 | −5.763 | 1.387 | 29.420 | 1.00 | 0.52 | 1SG | 1387 |
| ATOM | 1387 | C | LEU | 170 | −1.757 | 1.807 | 32.351 | 1.00 | 0.52 | 1SG | 1388 |
| ATOM | 1388 | O | LEU | 170 | −1.990 | 1.244 | 33.420 | 1.00 | 0.52 | 1SG | 1389 |
| ATOM | 1389 | N | LEU | 171 | −1.074 | 2.961 | 32.297 | 1.00 | 0.56 | 1SG | 1390 |
| ATOM | 1390 | CA | LEU | 171 | −0.610 | 3.460 | 33.548 | 1.00 | 0.56 | 1SG | 1391 |
| ATOM | 1391 | CB | LEU | 171 | 0.832 | 3.996 | 33.520 | 1.00 | 0.56 | 1SG | 1392 |
| ATOM | 1392 | CG | LEU | 171 | 1.880 | 2.920 | 33.173 | 1.00 | 0.56 | 1SG | 1393 |
| ATOM | 1393 | CD2 | LEU | 171 | 1.726 | 2.443 | 31.721 | 1.00 | 0.56 | 1SG | 1394 |
| ATOM | 1394 | CD1 | LEU | 171 | 1.886 | 1.777 | 34.205 | 1.00 | 0.56 | 1SG | 1395 |
| ATOM | 1395 | C | LEU | 171 | −1.491 | 4.571 | 33.966 | 1.00 | 0.56 | 1SG | 1396 |
| ATOM | 1396 | O | LEU | 171 | −1.653 | 5.567 | 33.261 | 1.00 | 0.56 | 1SG | 1397 |
| ATOM | 1397 | N | LYS | 172 | −2.122 | 4.396 | 35.137 | 1.00 | 0.80 | 1SG | 1398 |
| ATOM | 1398 | CA | LYS | 172 | −2.909 | 5.469 | 35.632 | 1.00 | 0.80 | 1SG | 1399 |
| ATOM | 1399 | CB | LYS | 172 | −4.384 | 5.112 | 35.852 | 1.00 | 0.80 | 1SG | 1400 |
| ATOM | 1400 | CG | LYS | 172 | −5.230 | 6.367 | 36.035 | 1.00 | 0.80 | 1SG | 1401 |
| ATOM | 1401 | CD | LYS | 172 | −6.727 | 6.135 | 35.879 | 1.00 | 0.80 | 1SG | 1402 |
| ATOM | 1402 | CE | LYS | 172 | −7.484 | 7.448 | 35.700 | 1.00 | 0.80 | 1SG | 1403 |
| ATOM | 1403 | NZ | LYS | 172 | −7.307 | 8.314 | 36.889 | 1.00 | 0.80 | 1SG | 1404 |
| ATOM | 1404 | C | LYS | 172 | −2.329 | 5.846 | 36.950 | 1.00 | 0.80 | 1SG | 1405 |
| ATOM | 1405 | O | LYS | 172 | −2.988 | 6.497 | 37.759 | 1.00 | 0.80 | 1SG | 1406 |
| ATOM | 1406 | N | LYS | 173 | −1.063 | 5.445 | 37.194 | 1.00 | 0.82 | 1SG | 1407 |
| ATOM | 1407 | CA | LYS | 173 | −0.427 | 5.848 | 38.411 | 1.00 | 0.82 | 1SG | 1408 |
| ATOM | 1408 | CB | LYS | 173 | 1.048 | 5.426 | 38.500 | 1.00 | 0.82 | 1SG | 1409 |
| ATOM | 1409 | CG | LYS | 173 | 1.720 | 5.928 | 39.779 | 1.00 | 0.82 | 1SG | 1410 |
| ATOM | 1410 | CD | LYS | 173 | 3.115 | 5.356 | 40.031 | 1.00 | 0.82 | 1SG | 1411 |
| ATOM | 1411 | CE | LYS | 173 | 3.817 | 6.015 | 41.220 | 1.00 | 0.82 | 1SG | 1412 |
| ATOM | 1412 | NZ | LYS | 173 | 4.053 | 7.449 | 40.933 | 1.00 | 0.82 | 1SG | 1413 |
| ATOM | 1413 | C | LYS | 173 | −0.438 | 7.324 | 38.325 | 1.00 | 0.82 | 1SG | 1414 |
| ATOM | 1414 | O | LYS | 173 | −0.886 | 8.026 | 39.231 | 1.00 | 0.82 | 1SG | 1415 |
| ATOM | 1415 | N | ASN | 174 | 0.052 | 7.821 | 37.181 | 1.00 | 0.73 | 1SG | 1416 |
| ATOM | 1416 | CA | ASN | 174 | −0.041 | 9.214 | 36.926 | 1.00 | 0.73 | 1SG | 1417 |
| ATOM | 1417 | CB | ASN | 174 | 1.210 | 9.804 | 36.248 | 1.00 | 0.73 | 1SG | 1418 |
| ATOM | 1418 | CG | ASN | 174 | 2.365 | 9.758 | 37.243 | 1.00 | 0.73 | 1SG | 1419 |
| ATOM | 1419 | OD1 | ASN | 174 | 3.531 | 9.741 | 36.851 | 1.00 | 0.73 | 1SG | 1420 |
| ATOM | 1420 | ND2 | ASN | 174 | 2.038 | 9.733 | 38.563 | 1.00 | 0.73 | 1SG | 1421 |
| ATOM | 1421 | C | ASN | 174 | −1.177 | 9.317 | 35.968 | 1.00 | 0.73 | 1SG | 1422 |
| ATOM | 1422 | O | ASN | 174 | −0.998 | 9.085 | 34.773 | 1.00 | 0.73 | 1SG | 1423 |
| ATOM | 1423 | N | ARG | 175 | −2.383 | 9.617 | 36.499 | 1.00 | 0.67 | 1SG | 1424 |
| ATOM | 1424 | CA | ARG | 175 | −3.568 | 9.799 | 35.712 | 1.00 | 0.67 | 1SG | 1425 |
| ATOM | 1425 | CB | ARG | 175 | −4.686 | 10.472 | 36.528 | 1.00 | 0.67 | 1SG | 1426 |
| ATOM | 1426 | CG | ARG | 175 | −6.003 | 10.692 | 35.787 | 1.00 | 0.67 | 1SG | 1427 |
| ATOM | 1427 | CD | ARG | 175 | −6.763 | 11.925 | 36.287 | 1.00 | 0.67 | 1SG | 1428 |
| ATOM | 1428 | NE | ARG | 175 | −6.997 | 11.789 | 37.753 | 1.00 | 0.67 | 1SG | 1429 |
| ATOM | 1429 | CZ | ARG | 175 | −8.194 | 11.321 | 38.213 | 1.00 | 0.67 | 1SG | 1430 |
| ATOM | 1430 | NH1 | ARG | 175 | −9.142 | 10.895 | 37.329 | 1.00 | 0.67 | 1SG | 1431 |
| ATOM | 1431 | NH2 | ARG | 175 | −8.444 | 11.293 | 39.554 | 1.00 | 0.67 | 1SG | 1432 |
| ATOM | 1432 | C | ARG | 175 | −3.145 | 10.754 | 34.653 | 1.00 | 0.67 | 1SG | 1433 |
| ATOM | 1433 | O | ARG | 175 | −2.614 | 11.818 | 34.959 | 1.00 | 0.67 | 1SG | 1434 |
| ATOM | 1434 | N | THR | 176 | −3.336 | 10.400 | 33.370 | 1.00 | 0.50 | 1SG | 1435 |
| ATOM | 1435 | CA | THR | 176 | −2.781 | 11.271 | 32.382 | 1.00 | 0.50 | 1SG | 1436 |
| ATOM | 1436 | CB | THR | 176 | −2.631 | 10.653 | 31.030 | 1.00 | 0.50 | 1SG | 1437 |
| ATOM | 1437 | OG1 | THR | 176 | −1.830 | 9.486 | 31.116 | 1.00 | 0.50 | 1SG | 1438 |
| ATOM | 1438 | CG2 | THR | 176 | −1.922 | 11.680 | 30.135 | 1.00 | 0.50 | 1SG | 1439 |
| ATOM | 1439 | C | THR | 176 | −3.653 | 12.465 | 32.227 | 1.00 | 0.50 | 1SG | 1440 |
| ATOM | 1440 | O | THR | 176 | −4.662 | 12.435 | 31.524 | 1.00 | 0.50 | 1SG | 1441 |
| ATOM | 1441 | N | THR | 177 | −3.271 | 13.557 | 32.911 | 1.00 | 0.49 | 1SG | 1442 |
| ATOM | 1442 | CA | THR | 177 | −3.992 | 14.785 | 32.818 | 1.00 | 0.49 | 1SG | 1443 |
| ATOM | 1443 | CB | THR | 177 | −3.596 | 15.782 | 33.868 | 1.00 | 0.49 | 1SG | 1444 |
| ATOM | 1444 | OG1 | THR | 177 | −4.482 | 16.892 | 33.847 | 1.00 | 0.49 | 1SG | 1445 |
| ATOM | 1445 | CG2 | THR | 177 | −2.154 | 16.247 | 33.600 | 1.00 | 0.49 | 1SG | 1446 |
| ATOM | 1446 | C | THR | 177 | −3.693 | 15.393 | 31.485 | 1.00 | 0.49 | 1SG | 1447 |
| ATOM | 1447 | O | THR | 177 | −4.553 | 16.023 | 30.872 | 1.00 | 0.49 | 1SG | 1448 |
| ATOM | 1448 | N | ARG | 178 | −2.456 | 15.198 | 30.993 | 1.00 | 0.55 | 1SG | 1449 |
| ATOM | 1449 | CA | ARG | 178 | −2.047 | 15.835 | 29.778 | 1.00 | 0.55 | 1SG | 1450 |
| ATOM | 1450 | CB | ARG | 178 | −0.563 | 15.641 | 29.439 | 1.00 | 0.55 | 1SG | 1451 |
| ATOM | 1451 | CG | ARG | 178 | −0.093 | 16.567 | 28.318 | 1.00 | 0.55 | 1SG | 1452 |
| ATOM | 1452 | CD | ARG | 178 | −0.028 | 18.042 | 28.713 | 1.00 | 0.55 | 1SG | 1453 |
| ATOM | 1453 | NE | ARG | 178 | 0.475 | 18.738 | 27.527 | 1.00 | 0.55 | 1SG | 1454 |
| ATOM | 1454 | CZ | ARG | 178 | 1.820 | 18.891 | 27.317 | 1.00 | 0.55 | 1SG | 1455 |
| ATOM | 1455 | NH1 | ARG | 178 | 2.690 | 18.301 | 28.187 | 1.00 | 0.55 | 1SG | 1456 |
| ATOM | 1456 | NH2 | ARG | 178 | 2.290 | 19.579 | 26.236 | 1.00 | 0.55 | 1SG | 1457 |
| ATOM | 1457 | C | ARG | 178 | −2.870 | 15.354 | 28.623 | 1.00 | 0.55 | 1SG | 1458 |
| ATOM | 1458 | O | ARG | 178 | −3.310 | 14.208 | 28.558 | 1.00 | 0.55 | 1SG | 1459 |
| ATOM | 1459 | N | PHE | 179 | −3.055 | 16.285 | 27.671 | 1.00 | 0.63 | 1SG | 1460 |
| ATOM | 1460 | CA | PHE | 179 | −3.848 | 16.214 | 26.478 | 1.00 | 0.63 | 1SG | 1461 |
| ATOM | 1461 | CB | PHE | 179 | −3.775 | 17.557 | 25.730 | 1.00 | 0.63 | 1SG | 1462 |
| ATOM | 1462 | CG | PHE | 179 | −4.570 | 17.499 | 24.478 | 1.00 | 0.63 | 1SG | 1463 |
| ATOM | 1463 | CD1 | PHE | 179 | −5.921 | 17.764 | 24.487 | 1.00 | 0.63 | 1SG | 1464 |
| ATOM | 1464 | CD2 | PHE | 179 | −3.952 | 17.182 | 23.295 | 1.00 | 0.63 | 1SG | 1465 |
| ATOM | 1465 | CE1 | PHE | 179 | −6.642 | 17.711 | 23.318 | 1.00 | 0.63 | 1SG | 1466 |
| ATOM | 1466 | CE2 | PHE | 179 | −4.665 | 17.128 | 22.123 | 1.00 | 0.63 | 1SG | 1467 |
| ATOM | 1467 | CZ | PHE | 179 | −6.012 | 17.394 | 22.139 | 1.00 | 0.63 | 1SG | 1468 |
| ATOM | 1468 | C | PHE | 179 | −3.414 | 15.107 | 25.568 | 1.00 | 0.63 | 1SG | 1469 |
| ATOM | 1469 | O | PHE | 179 | −4.077 | 14.077 | 25.483 | 1.00 | 0.63 | 1SG | 1470 |
| ATOM | 1470 | N | PHE | 180 | −2.280 | 15.293 | 24.865 | 1.00 | 0.88 | 1SG | 1471 |
| ATOM | 1471 | CA | PHE | 180 | −1.767 | 14.388 | 23.874 | 1.00 | 0.88 | 1SG | 1472 |
| ATOM | 1472 | CB | PHE | 180 | −0.948 | 13.212 | 24.427 | 1.00 | 0.88 | 1SG | 1473 |
| ATOM | 1473 | CG | PHE | 180 | 0.219 | 13.927 | 25.002 | 1.00 | 0.88 | 1SG | 1474 |
| ATOM | 1474 | CD1 | PHE | 180 | 1.081 | 14.613 | 24.175 | 1.00 | 0.88 | 1SG | 1475 |
| ATOM | 1475 | CD2 | PHE | 180 | 0.452 | 13.910 | 26.355 | 1.00 | 0.88 | 1SG | 1476 |
| ATOM | 1476 | CE1 | PHE | 180 | 2.155 | 15.292 | 24.695 | 1.00 | 0.88 | 1SG | 1477 |
| ATOM | 1477 | CE2 | PHE | 180 | 1.526 | 14.584 | 26.878 | 1.00 | 0.88 | 1SG | 1478 |
| ATOM | 1478 | CZ | PHE | 180 | 2.372 | 15.280 | 26.051 | 1.00 | 0.88 | 1SG | 1479 |
| ATOM | 1479 | C | PHE | 180 | −2.841 | 13.940 | 22.936 | 1.00 | 0.88 | 1SG | 1480 |
| ATOM | 1480 | O | PHE | 180 | −3.949 | 14.464 | 22.958 | 1.00 | 0.88 | 1SG | 1481 |
| ATOM | 1481 | N | THR | 181 | −2.530 | 12.973 | 22.054 | 1.00 | 0.90 | 1SG | 1482 |
| ATOM | 1482 | CA | THR | 181 | −3.475 | 12.617 | 21.036 | 1.00 | 0.90 | 1SG | 1483 |
| ATOM | 1483 | CB | THR | 181 | −3.041 | 13.113 | 19.689 | 1.00 | 0.90 | 1SG | 1484 |
| ATOM | 1484 | OG1 | THR | 181 | −4.099 | 13.022 | 18.750 | 1.00 | 0.90 | 1SG | 1485 |
| ATOM | 1485 | CG2 | THR | 181 | −1.844 | 12.268 | 19.230 | 1.00 | 0.90 | 1SG | 1486 |
| ATOM | 1486 | C | THR | 181 | −3.585 | 11.120 | 20.973 | 1.00 | 0.90 | 1SG | 1487 |
| ATOM | 1487 | O | THR | 181 | −3.224 | 10.420 | 21.915 | 1.00 | 0.90 | 1SG | 1488 |
| ATOM | 1488 | N | GLY | 182 | −4.106 | 10.600 | 19.841 | 1.00 | 0.78 | 1SG | 1489 |
| ATOM | 1489 | CA | GLY | 182 | −4.346 | 9.202 | 19.628 | 1.00 | 0.78 | 1SG | 1490 |
| ATOM | 1490 | C | GLY | 182 | −5.813 | 9.137 | 19.426 | 1.00 | 0.78 | 1SG | 1491 |
| ATOM | 1491 | O | GLY | 182 | −6.291 | 8.597 | 18.432 | 1.00 | 0.78 | 1SG | 1492 |
| ATOM | 1492 | N | PHE | 183 | −6.557 | 9.647 | 20.424 | 1.00 | 0.82 | 1SG | 1493 |
| ATOM | 1493 | CA | PHE | 183 | −7.956 | 9.925 | 20.320 | 1.00 | 0.82 | 1SG | 1494 |
| ATOM | 1494 | CB | PHE | 183 | −8.464 | 10.186 | 18.895 | 1.00 | 0.82 | 1SG | 1495 |
| ATOM | 1495 | CG | PHE | 183 | −7.724 | 11.341 | 18.311 | 1.00 | 0.82 | 1SG | 1496 |
| ATOM | 1496 | CD1 | PHE | 183 | −8.079 | 12.631 | 18.619 | 1.00 | 0.82 | 1SG | 1497 |
| ATOM | 1497 | CD2 | PHE | 183 | −6.676 | 11.140 | 17.441 | 1.00 | 0.82 | 1SG | 1498 |
| ATOM | 1498 | CE1 | PHE | 183 | −7.405 | 13.695 | 18.070 | 1.00 | 0.82 | 1SG | 1499 |
| ATOM | 1499 | CE2 | PHE | 183 | −5.998 | 12.199 | 16.886 | 1.00 | 0.82 | 1SG | 1500 |
| ATOM | 1500 | CZ | PHE | 183 | −6.362 | 13.484 | 17.203 | 1.00 | 0.82 | 1SG | 1501 |
| ATOM | 1501 | C | PHE | 183 | −8.803 | 8.852 | 20.920 | 1.00 | 0.82 | 1SG | 1502 |
| ATOM | 1502 | O | PHE | 183 | −8.356 | 7.750 | 21.219 | 1.00 | 0.82 | 1SG | 1503 |
| ATOM | 1503 | N | LEU | 184 | −10.079 | 9.223 | 21.119 | 1.00 | 0.90 | 1SG | 1504 |
| ATOM | 1504 | CA | LEU | 184 | −11.233 | 8.501 | 21.572 | 1.00 | 0.90 | 1SG | 1505 |
| ATOM | 1505 | CB | LEU | 184 | −11.079 | 7.283 | 22.501 | 1.00 | 0.90 | 1SG | 1506 |
| ATOM | 1506 | CG | LEU | 184 | −11.203 | 5.931 | 21.760 | 1.00 | 0.90 | 1SG | 1507 |
| ATOM | 1507 | CD2 | LEU | 184 | −11.612 | 4.809 | 22.724 | 1.00 | 0.90 | 1SG | 1508 |
| ATOM | 1508 | CD1 | LEU | 184 | −9.996 | 5.589 | 20.881 | 1.00 | 0.90 | 1SG | 1509 |
| ATOM | 1509 | C | LEU | 184 | −12.119 | 9.489 | 22.212 | 1.00 | 0.90 | 1SG | 1510 |
| ATOM | 1510 | O | LEU | 184 | −11.667 | 10.370 | 22.942 | 1.00 | 0.90 | 1SG | 1511 |
| ATOM | 1511 | N | LYS | 185 | −13.426 | 9.332 | 21.962 | 1.00 | 0.92 | 1SG | 1512 |
| ATOM | 1512 | CA | LYS | 185 | −14.363 | 10.310 | 22.401 | 1.00 | 0.92 | 1SG | 1513 |
| ATOM | 1513 | CB | LYS | 185 | −14.828 | 11.187 | 21.243 | 1.00 | 0.92 | 1SG | 1514 |
| ATOM | 1514 | CG | LYS | 185 | −15.394 | 10.383 | 20.073 | 1.00 | 0.92 | 1SG | 1515 |
| ATOM | 1515 | CD | LYS | 185 | −16.115 | 11.248 | 19.042 | 1.00 | 0.92 | 1SG | 1516 |
| ATOM | 1516 | CE | LYS | 185 | −17.631 | 11.270 | 19.227 | 1.00 | 0.92 | 1SG | 1517 |
| ATOM | 1517 | NZ | LYS | 185 | −18.202 | 9.996 | 18.740 | 1.00 | 0.92 | 1SG | 1518 |
| ATOM | 1518 | C | LYS | 185 | −15.613 | 9.626 | 22.795 | 1.00 | 0.92 | 1SG | 1519 |
| ATOM | 1519 | O | LYS | 185 | −16.041 | 8.681 | 22.135 | 1.00 | 0.92 | 1SG | 1520 |
| ATOM | 1520 | N | LEU | 186 | −16.253 | 10.137 | 23.860 | 1.00 | 0.79 | 1SG | 1521 |
| ATOM | 1521 | CA | LEU | 186 | −17.534 | 9.635 | 24.239 | 1.00 | 0.79 | 1SG | 1522 |
| ATOM | 1522 | CB | LEU | 186 | −17.716 | 9.597 | 25.767 | 1.00 | 0.79 | 1SG | 1523 |
| ATOM | 1523 | CG | LEU | 186 | −18.781 | 8.602 | 26.262 | 1.00 | 0.79 | 1SG | 1524 |
| ATOM | 1524 | CD2 | LEU | 186 | −18.325 | 7.148 | 26.055 | 1.00 | 0.79 | 1SG | 1525 |
| ATOM | 1525 | CD1 | LEU | 186 | −20.159 | 8.896 | 25.661 | 1.00 | 0.79 | 1SG | 1526 |
| ATOM | 1526 | C | LEU | 186 | −18.434 | 10.687 | 23.671 | 1.00 | 0.79 | 1SG | 1527 |
| ATOM | 1527 | O | LEU | 186 | −18.028 | 11.840 | 23.593 | 1.00 | 0.79 | 1SG | 1528 |
| ATOM | 1528 | N | ASN | 187 | −19.666 | 10.354 | 23.256 | 1.00 | 0.55 | 1SG | 1529 |
| ATOM | 1529 | CA | ASN | 187 | −20.495 | 11.325 | 22.590 | 1.00 | 0.55 | 1SG | 1530 |
| ATOM | 1530 | CB | ASN | 187 | −21.888 | 10.770 | 22.247 | 1.00 | 0.55 | 1SG | 1531 |
| ATOM | 1531 | CG | ASN | 187 | −22.594 | 11.738 | 21.303 | 1.00 | 0.55 | 1SG | 1532 |
| ATOM | 1532 | OD1 | ASN | 187 | −22.142 | 12.857 | 21.067 | 1.00 | 0.55 | 1SG | 1533 |
| ATOM | 1533 | ND2 | ASN | 187 | −23.753 | 11.297 | 20.743 | 1.00 | 0.55 | 1SG | 1534 |
| ATOM | 1534 | C | ASN | 187 | −20.686 | 12.518 | 23.475 | 1.00 | 0.55 | 1SG | 1535 |
| ATOM | 1535 | O | ASN | 187 | −20.703 | 13.652 | 23.000 | 1.00 | 0.55 | 1SG | 1536 |
| ATOM | 1536 | N | GLU | 188 | −20.813 | 12.305 | 24.794 | 1.00 | 0.49 | 1SG | 1537 |
| ATOM | 1537 | CA | GLU | 188 | −21.098 | 13.396 | 25.681 | 1.00 | 0.49 | 1SG | 1538 |
| ATOM | 1538 | CB | GLU | 188 | −21.293 | 12.962 | 27.142 | 1.00 | 0.49 | 1SG | 1539 |
| ATOM | 1539 | CG | GLU | 188 | −22.693 | 12.409 | 27.419 | 1.00 | 0.49 | 1SG | 1540 |
| ATOM | 1540 | CD | GLU | 188 | −22.873 | 11.127 | 26.623 | 1.00 | 0.49 | 1SG | 1541 |
| ATOM | 1541 | OE1 | GLU | 188 | −22.225 | 10.112 | 26.987 | 1.00 | 0.49 | 1SG | 1542 |
| ATOM | 1542 | OE2 | GLU | 188 | −23.660 | 11.146 | 25.638 | 1.00 | 0.49 | 1SG | 1543 |
| ATOM | 1543 | C | GLU | 188 | −20.022 | 14.437 | 25.648 | 1.00 | 0.49 | 1SG | 1544 |
| ATOM | 1544 | O | GLU | 188 | −20.329 | 15.625 | 25.737 | 1.00 | 0.49 | 1SG | 1545 |
| ATOM | 1545 | N | PHE | 189 | −18.740 | 14.049 | 25.515 | 1.00 | 0.52 | 1SG | 1546 |
| ATOM | 1546 | CA | PHE | 189 | −17.717 | 15.055 | 25.588 | 1.00 | 0.52 | 1SG | 1547 |
| ATOM | 1547 | CB | PHE | 189 | −16.276 | 14.513 | 25.498 | 1.00 | 0.52 | 1SG | 1548 |
| ATOM | 1548 | CG | PHE | 189 | −15.390 | 15.678 | 25.782 | 1.00 | 0.52 | 1SG | 1549 |
| ATOM | 1549 | CD1 | PHE | 189 | −15.048 | 16.566 | 24.788 | 1.00 | 0.52 | 1SG | 1550 |
| ATOM | 1550 | CD2 | PHE | 189 | −14.906 | 15.886 | 27.052 | 1.00 | 0.52 | 1SG | 1551 |
| ATOM | 1551 | CE1 | PHE | 189 | −14.243 | 17.645 | 25.052 | 1.00 | 0.52 | 1SG | 1552 |
| ATOM | 1552 | CE2 | PHE | 189 | −14.097 | 16.966 | 27.320 | 1.00 | 0.52 | 1SG | 1553 |
| ATOM | 1553 | CZ | PHE | 189 | −13.764 | 17.850 | 26.322 | 1.00 | 0.52 | 1SG | 1554 |
| ATOM | 1554 | C | PHE | 189 | −17.923 | 16.057 | 24.491 | 1.00 | 0.52 | 1SG | 1555 |
| ATOM | 1555 | O | PHE | 189 | −17.761 | 17.258 | 24.703 | 1.00 | 0.52 | 1SG | 1556 |
| ATOM | 1556 | N | PRO | 190 | −18.285 | 15.610 | 23.327 | 1.00 | 0.57 | 1SG | 1557 |
| ATOM | 1557 | CA | PRO | 190 | −18.525 | 16.539 | 22.271 | 1.00 | 0.57 | 1SG | 1558 |
| ATOM | 1558 | CD | PRO | 190 | −17.586 | 14.473 | 22.773 | 1.00 | 0.57 | 1SG | 1559 |
| ATOM | 1559 | CB | PRO | 190 | −18.705 | 15.685 | 21.016 | 1.00 | 0.57 | 1SG | 1560 |
| ATOM | 1560 | CG | PRO | 190 | −18.283 | 14.263 | 21.428 | 1.00 | 0.57 | 1SG | 1561 |
| ATOM | 1561 | C | PRO | 190 | −19.657 | 17.459 | 22.571 | 1.00 | 0.57 | 1SG | 1562 |
| ATOM | 1562 | O | PRO | 190 | −19.821 | 18.438 | 21.847 | 1.00 | 0.57 | 1SG | 1563 |
| ATOM | 1563 | N | ILE | 191 | −20.440 | 17.193 | 23.628 | 1.00 | 0.63 | 1SG | 1564 |
| ATOM | 1564 | CA | ILE | 191 | −21.536 | 18.067 | 23.928 | 1.00 | 0.63 | 1SG | 1565 |
| ATOM | 1565 | CB | ILE | 191 | −22.279 | 17.703 | 25.181 | 1.00 | 0.63 | 1SG | 1566 |
| ATOM | 1566 | CG2 | ILE | 191 | −21.392 | 18.069 | 26.384 | 1.00 | 0.63 | 1SG | 1567 |
| ATOM | 1567 | CG1 | ILE | 191 | −23.642 | 18.416 | 25.207 | 1.00 | 0.63 | 1SG | 1568 |
| ATOM | 1568 | CD1 | ILE | 191 | −24.600 | 17.946 | 24.113 | 1.00 | 0.63 | 1SG | 1569 |
| ATOM | 1569 | C | ILE | 191 | −20.953 | 19.430 | 24.141 | 1.00 | 0.63 | 1SG | 1570 |
| ATOM | 1570 | O | ILE | 191 | −21.613 | 20.440 | 23.917 | 1.00 | 0.63 | 1SG | 1571 |
| ATOM | 1571 | N | ARG | 192 | −19.687 | 19.473 | 24.598 | 1.00 | 0.68 | 1SG | 1572 |
| ATOM | 1572 | CA | ARG | 192 | −18.936 | 20.666 | 24.884 | 1.00 | 0.68 | 1SG | 1573 |
| ATOM | 1573 | CB | ARG | 192 | −17.570 | 20.379 | 25.532 | 1.00 | 0.68 | 1SG | 1574 |
| ATOM | 1574 | CG | ARG | 192 | −17.676 | 19.965 | 27.001 | 1.00 | 0.68 | 1SG | 1575 |
| ATOM | 1575 | CD | ARG | 192 | −16.328 | 19.686 | 27.667 | 1.00 | 0.68 | 1SG | 1576 |
| ATOM | 1576 | NE | ARG | 192 | −16.580 | 19.540 | 29.128 | 1.00 | 0.68 | 1SG | 1577 |
| ATOM | 1577 | CZ | ARG | 192 | −16.533 | 20.643 | 29.932 | 1.00 | 0.68 | 1SG | 1578 |
| ATOM | 1578 | NH1 | ARG | 192 | −16.242 | 21.864 | 29.395 | 1.00 | 0.68 | 1SG | 1579 |
| ATOM | 1579 | NH2 | ARG | 192 | −16.776 | 20.525 | 31.270 | 1.00 | 0.68 | 1SG | 1580 |
| ATOM | 1580 | C | ARG | 192 | −18.682 | 21.446 | 23.625 | 1.00 | 0.68 | 1SG | 1581 |
| ATOM | 1581 | O | ARG | 192 | −18.349 | 22.628 | 23.679 | 1.00 | 0.68 | 1SG | 1582 |
| ATOM | 1582 | N | GLN | 193 | −18.843 | 20.791 | 22.460 | 1.00 | 0.91 | 1SG | 1583 |
| ATOM | 1583 | CA | GLN | 193 | −18.521 | 21.281 | 21.143 | 1.00 | 0.91 | 1SG | 1584 |
| ATOM | 1584 | CB | GLN | 193 | −19.005 | 20.337 | 20.047 | 1.00 | 0.91 | 1SG | 1585 |
| ATOM | 1585 | CG | GLN | 193 | −18.597 | 20.768 | 18.641 | 1.00 | 0.91 | 1SG | 1586 |
| ATOM | 1586 | CD | GLN | 193 | −17.124 | 20.430 | 18.493 | 1.00 | 0.91 | 1SG | 1587 |
| ATOM | 1587 | OE1 | GLN | 193 | −16.449 | 20.113 | 19.471 | 1.00 | 0.91 | 1SG | 1588 |
| ATOM | 1588 | NE2 | GLN | 193 | −16.620 | 20.472 | 17.233 | 1.00 | 0.91 | 1SG | 1589 |
| ATOM | 1589 | C | GLN | 193 | −19.101 | 22.617 | 20.750 | 1.00 | 0.91 | 1SG | 1590 |
| ATOM | 1590 | O | GLN | 193 | −18.324 | 23.353 | 20.143 | 1.00 | 0.91 | 1SG | 1591 |
| ATOM | 1591 | N | PRO | 194 | −20.354 | 22.999 | 20.984 | 1.00 | 1.01 | 1SG | 1592 |
| ATOM | 1592 | CA | PRO | 194 | −20.904 | 24.239 | 20.468 | 1.00 | 1.01 | 1SG | 1593 |
| ATOM | 1593 | CD | PRO | 194 | −21.078 | 22.588 | 22.176 | 1.00 | 1.01 | 1SG | 1594 |
| ATOM | 1594 | CB | PRO | 194 | −22.218 | 24.474 | 21.209 | 1.00 | 1.01 | 1SG | 1595 |
| ATOM | 1595 | CG | PRO | 194 | −22.003 | 23.756 | 22.545 | 1.00 | 1.01 | 1SG | 1596 |
| ATOM | 1596 | C | PRO | 194 | −20.009 | 25.430 | 20.580 | 1.00 | 1.01 | 1SG | 1597 |
| ATOM | 1597 | O | PRO | 194 | −19.081 | 25.416 | 21.387 | 1.00 | 1.01 | 1SG | 1598 |
| ATOM | 1598 | N | PHE | 195 | −20.286 | 26.468 | 19.764 | 1.00 | 0.79 | 1SG | 1599 |
| ATOM | 1599 | CA | PHE | 195 | −19.461 | 27.637 | 19.685 | 1.00 | 0.79 | 1SG | 1600 |
| ATOM | 1600 | CB | PHE | 195 | −19.816 | 28.497 | 18.454 | 1.00 | 0.79 | 1SG | 1601 |
| ATOM | 1601 | CG | PHE | 195 | −18.783 | 29.556 | 18.251 | 1.00 | 0.79 | 1SG | 1602 |
| ATOM | 1602 | CD1 | PHE | 195 | −17.527 | 29.221 | 17.799 | 1.00 | 0.79 | 1SG | 1603 |
| ATOM | 1603 | CD2 | PHE | 195 | −19.076 | 30.883 | 18.471 | 1.00 | 0.79 | 1SG | 1604 |
| ATOM | 1604 | CE1 | PHE | 195 | −16.571 | 30.189 | 17.600 | 1.00 | 0.79 | 1SG | 1605 |
| ATOM | 1605 | CE2 | PHE | 195 | −18.124 | 31.855 | 18.274 | 1.00 | 0.79 | 1SG | 1606 |
| ATOM | 1606 | CZ | PHE | 195 | −16.867 | 31.509 | 17.841 | 1.00 | 0.79 | 1SG | 1607 |
| ATOM | 1607 | C | PHE | 195 | −19.631 | 28.460 | 20.924 | 1.00 | 0.79 | 1SG | 1608 |
| ATOM | 1608 | O | PHE | 195 | −19.565 | 29.686 | 20.882 | 1.00 | 0.79 | 1SG | 1609 |
| ATOM | 1609 | N | SER | 196 | −19.853 | 27.809 | 22.078 | 1.00 | 0.60 | 1SG | 1610 |
| ATOM | 1610 | CA | SER | 196 | −19.879 | 28.551 | 23.295 | 1.00 | 0.60 | 1SG | 1611 |
| ATOM | 1611 | CB | SER | 196 | −20.204 | 27.680 | 24.520 | 1.00 | 0.60 | 1SG | 1612 |
| ATOM | 1612 | OG | SER | 196 | −20.216 | 28.476 | 25.696 | 1.00 | 0.60 | 1SG | 1613 |
| ATOM | 1613 | C | SER | 196 | −18.477 | 29.029 | 23.434 | 1.00 | 0.60 | 1SG | 1614 |
| ATOM | 1614 | O | SER | 196 | −18.223 | 30.200 | 23.710 | 1.00 | 0.60 | 1SG | 1615 |
| ATOM | 1615 | N | LYS | 197 | −17.526 | 28.104 | 23.213 | 1.00 | 0.48 | 1SG | 1616 |
| ATOM | 1616 | CA | LYS | 197 | −16.144 | 28.462 | 23.266 | 1.00 | 0.48 | 1SG | 1617 |
| ATOM | 1617 | CB | LYS | 197 | −15.492 | 28.164 | 24.626 | 1.00 | 0.48 | 1SG | 1618 |
| ATOM | 1618 | CG | LYS | 197 | −14.105 | 28.791 | 24.786 | 1.00 | 0.48 | 1SG | 1619 |
| ATOM | 1619 | CD | LYS | 197 | −13.606 | 28.823 | 26.231 | 1.00 | 0.48 | 1SG | 1620 |
| ATOM | 1620 | CE | LYS | 197 | −12.226 | 29.465 | 26.391 | 1.00 | 0.48 | 1SG | 1621 |
| ATOM | 1621 | NZ | LYS | 197 | −11.838 | 29.490 | 27.819 | 1.00 | 0.48 | 1SG | 1622 |
| ATOM | 1622 | C | LYS | 197 | −15.449 | 27.638 | 22.233 | 1.00 | 0.48 | 1SG | 1623 |
| ATOM | 1623 | O | LYS | 197 | −14.224 | 27.536 | 22.222 | 1.00 | 0.48 | 1SG | 1624 |
| ATOM | 1624 | N | TRP | 198 | −16.231 | 27.036 | 21.318 | 1.00 | 0.45 | 1SG | 1625 |
| ATOM | 1625 | CA | TRP | 198 | −15.660 | 26.210 | 20.299 | 1.00 | 0.45 | 1SG | 1626 |
| ATOM | 1626 | CB | TRP | 198 | −16.716 | 25.512 | 19.424 | 1.00 | 0.45 | 1SG | 1627 |
| ATOM | 1627 | CG | TRP | 198 | −16.127 | 24.689 | 18.310 | 1.00 | 0.45 | 1SG | 1628 |
| ATOM | 1628 | CD2 | TRP | 198 | −15.353 | 23.507 | 18.537 | 1.00 | 0.45 | 1SG | 1629 |
| ATOM | 1629 | CD1 | TRP | 198 | −16.134 | 24.896 | 16.961 | 1.00 | 0.45 | 1SG | 1630 |
| ATOM | 1630 | NE1 | TRP | 198 | −15.425 | 23.901 | 16.332 | 1.00 | 0.45 | 1SG | 1631 |
| ATOM | 1631 | CE2 | TRP | 198 | −14.932 | 23.042 | 17.292 | 1.00 | 0.45 | 1SG | 1632 |
| ATOM | 1632 | CE3 | TRP | 198 | −15.015 | 22.867 | 19.694 | 1.00 | 0.45 | 1SG | 1633 |
| ATOM | 1633 | CZ2 | TRP | 198 | −14.162 | 21.922 | 17.185 | 1.00 | 0.45 | 1SG | 1634 |
| ATOM | 1634 | CZ3 | TRP | 198 | −14.244 | 21.737 | 19.582 | 1.00 | 0.45 | 1SG | 1635 |
| ATOM | 1635 | CH2 | TRP | 198 | −13.826 | 21.273 | 18.352 | 1.00 | 0.45 | 1SG | 1636 |
| ATOM | 1636 | C | TRP | 198 | −14.810 | 27.070 | 19.428 | 1.00 | 0.45 | 1SG | 1637 |
| ATOM | 1637 | O | TRP | 198 | −15.167 | 28.201 | 19.106 | 1.00 | 0.45 | 1SG | 1638 |
| ATOM | 1638 | N | PHE | 199 | −13.636 | 26.545 | 19.029 | 1.00 | 0.56 | 1SG | 1639 |
| ATOM | 1639 | CA | PHE | 199 | −12.766 | 27.327 | 18.211 | 1.00 | 0.56 | 1SG | 1640 |
| ATOM | 1640 | CB | PHE | 199 | −11.353 | 26.744 | 18.069 | 1.00 | 0.56 | 1SG | 1641 |
| ATOM | 1641 | CG | PHE | 199 | −11.434 | 25.405 | 17.416 | 1.00 | 0.56 | 1SG | 1642 |
| ATOM | 1642 | CD1 | PHE | 199 | −11.531 | 25.276 | 16.048 | 1.00 | 0.56 | 1SG | 1643 |
| ATOM | 1643 | CD2 | PHE | 199 | −11.402 | 24.266 | 18.184 | 1.00 | 0.56 | 1SG | 1644 |
| ATOM | 1644 | CE1 | PHE | 199 | −11.594 | 24.040 | 15.453 | 1.00 | 0.56 | 1SG | 1645 |
| ATOM | 1645 | CE2 | PHE | 199 | −11.464 | 23.028 | 17.593 | 1.00 | 0.56 | 1SG | 1646 |
| ATOM | 1646 | CZ | PHE | 199 | −11.560 | 22.909 | 16.228 | 1.00 | 0.56 | 1SG | 1647 |
| ATOM | 1647 | C | PHE | 199 | −13.379 | 27.427 | 16.861 | 1.00 | 0.56 | 1SG | 1648 |
| ATOM | 1648 | O | PHE | 199 | −13.975 | 26.478 | 16.355 | 1.00 | 0.56 | 1SG | 1649 |
| ATOM | 1649 | N | VAL | 200 | −13.253 | 28.612 | 16.242 | 1.00 | 0.61 | 1SG | 1650 |
| ATOM | 1650 | CA | VAL | 200 | −13.833 | 28.818 | 14.954 | 1.00 | 0.61 | 1SG | 1651 |
| ATOM | 1651 | CB | VAL | 200 | −14.464 | 30.178 | 14.819 | 1.00 | 0.61 | 1SG | 1652 |
| ATOM | 1652 | CG1 | VAL | 200 | −13.346 | 31.231 | 14.759 | 1.00 | 0.61 | 1SG | 1653 |
| ATOM | 1653 | CG2 | VAL | 200 | −15.428 | 30.187 | 13.619 | 1.00 | 0.61 | 1SG | 1654 |
| ATOM | 1654 | C | VAL | 200 | −12.712 | 28.706 | 13.974 | 1.00 | 0.61 | 1SG | 1655 |
| ATOM | 1655 | O | VAL | 200 | −11.675 | 28.114 | 14.268 | 1.00 | 0.61 | 1SG | 1656 |
| ATOM | 1656 | N | SER | 201 | −12.902 | 29.269 | 12.770 | 1.00 | 0.53 | 1SG | 1657 |
| ATOM | 1657 | CA | SER | 201 | −11.891 | 29.224 | 11.761 | 1.00 | 0.53 | 1SG | 1658 |
| ATOM | 1658 | CB | SER | 201 | −12.293 | 29.981 | 10.484 | 1.00 | 0.53 | 1SG | 1659 |
| ATOM | 1659 | OG | SER | 201 | −13.417 | 29.358 | 9.879 | 1.00 | 0.53 | 1SG | 1660 |
| ATOM | 1660 | C | SER | 201 | −10.700 | 29.909 | 12.337 | 1.00 | 0.53 | 1SG | 1661 |
| ATOM | 1661 | O | SER | 201 | −9.561 | 29.632 | 11.966 | 1.00 | 0.53 | 1SG | 1662 |
| ATOM | 1662 | N | LYS | 202 | −10.954 | 30.816 | 13.294 | 1.00 | 0.41 | 1SG | 1663 |
| ATOM | 1663 | CA | LYS | 202 | −9.916 | 31.594 | 13.894 | 1.00 | 0.41 | 1SG | 1664 |
| ATOM | 1664 | CB | LYS | 202 | −10.439 | 32.575 | 14.957 | 1.00 | 0.41 | 1SG | 1665 |
| ATOM | 1665 | CG | LYS | 202 | −11.314 | 33.693 | 14.385 | 1.00 | 0.41 | 1SG | 1666 |
| ATOM | 1666 | CD | LYS | 202 | −12.075 | 34.482 | 15.454 | 1.00 | 0.41 | 1SG | 1667 |
| ATOM | 1667 | CE | LYS | 202 | −12.951 | 35.603 | 14.887 | 1.00 | 0.41 | 1SG | 1668 |
| ATOM | 1668 | NZ | LYS | 202 | −14.161 | 35.030 | 14.257 | 1.00 | 0.41 | 1SG | 1669 |
| ATOM | 1669 | C | LYS | 202 | −8.941 | 30.686 | 14.568 | 1.00 | 0.41 | 1SG | 1670 |
| ATOM | 1670 | O | LYS | 202 | −7.743 | 30.958 | 14.532 | 1.00 | 0.41 | 1SG | 1671 |
| ATOM | 1671 | N | SER | 203 | −9.450 | 29.590 | 15.177 | 1.00 | 0.49 | 1SG | 1672 |
| ATOM | 1672 | CA | SER | 203 | −8.716 | 28.629 | 15.965 | 1.00 | 0.49 | 1SG | 1673 |
| ATOM | 1673 | CB | SER | 203 | −9.342 | 27.233 | 15.976 | 1.00 | 0.49 | 1SG | 1674 |
| ATOM | 1674 | OG | SER | 203 | −9.310 | 26.673 | 14.671 | 1.00 | 0.49 | 1SG | 1675 |
| ATOM | 1675 | C | SER | 203 | −7.322 | 28.453 | 15.481 | 1.00 | 0.49 | 1SG | 1676 |
| ATOM | 1676 | O | SER | 203 | −7.076 | 28.249 | 14.294 | 1.00 | 0.49 | 1SG | 1677 |
| ATOM | 1677 | N | GLU | 204 | −6.368 | 28.538 | 16.423 | 1.00 | 0.73 | 1SG | 1678 |
| ATOM | 1678 | CA | GLU | 204 | −4.988 | 28.409 | 16.079 | 1.00 | 0.73 | 1SG | 1679 |
| ATOM | 1679 | CB | GLU | 204 | −4.033 | 28.800 | 17.219 | 1.00 | 0.73 | 1SG | 1680 |
| ATOM | 1680 | CG | GLU | 204 | −4.215 | 27.953 | 18.481 | 1.00 | 0.73 | 1SG | 1681 |
| ATOM | 1681 | CD | GLU | 204 | −3.214 | 28.433 | 19.522 | 1.00 | 0.73 | 1SG | 1682 |
| ATOM | 1682 | OE1 | GLU | 204 | −2.438 | 29.374 | 19.207 | 1.00 | 0.73 | 1SG | 1683 |
| ATOM | 1683 | OE2 | GLU | 204 | −3.214 | 27.865 | 20.647 | 1.00 | 0.73 | 1SG | 1684 |
| ATOM | 1684 | C | GLU | 204 | −4.738 | 26.980 | 15.751 | 1.00 | 0.73 | 1SG | 1685 |
| ATOM | 1685 | O | GLU | 204 | −5.662 | 26.209 | 15.493 | 1.00 | 0.73 | 1SG | 1686 |
| ATOM | 1686 | N | TYR | 205 | −3.447 | 26.603 | 15.740 | 1.00 | 0.73 | 1SG | 1687 |
| ATOM | 1687 | CA | TYR | 205 | −3.050 | 25.270 | 15.415 | 1.00 | 0.73 | 1SG | 1688 |
| ATOM | 1688 | CB | TYR | 205 | −1.513 | 25.139 | 15.355 | 1.00 | 0.73 | 1SG | 1689 |
| ATOM | 1689 | CG | TYR | 205 | −1.113 | 23.744 | 15.023 | 1.00 | 0.73 | 1SG | 1690 |
| ATOM | 1690 | CD1 | TYR | 205 | −1.024 | 23.335 | 13.710 | 1.00 | 0.73 | 1SG | 1691 |
| ATOM | 1691 | CD2 | TYR | 205 | −0.814 | 22.849 | 16.022 | 1.00 | 0.73 | 1SG | 1692 |
| ATOM | 1692 | CE1 | TYR | 205 | −0.645 | 22.048 | 13.404 | 1.00 | 0.73 | 1SG | 1693 |
| ATOM | 1693 | CE2 | TYR | 205 | −0.437 | 21.564 | 15.723 | 1.00 | 0.73 | 1SG | 1694 |
| ATOM | 1694 | CZ | TYR | 205 | −0.352 | 21.162 | 14.411 | 1.00 | 0.73 | 1SG | 1695 |
| ATOM | 1695 | OH | TYR | 205 | 0.036 | 19.842 | 14.098 | 1.00 | 0.73 | 1SG | 1696 |
| ATOM | 1696 | C | TYR | 205 | −3.623 | 24.349 | 16.456 | 1.00 | 0.73 | 1SG | 1697 |
| ATOM | 1697 | O | TYR | 205 | −4.155 | 23.294 | 16.111 | 1.00 | 0.73 | 1SG | 1698 |
| ATOM | 1698 | N | PRO | 206 | −3.548 | 24.697 | 17.714 | 1.00 | 0.57 | 1SG | 1699 |
| ATOM | 1699 | CA | PRO | 206 | −4.109 | 23.814 | 18.699 | 1.00 | 0.57 | 1SG | 1700 |
| ATOM | 1700 | CD | PRO | 206 | −2.379 | 25.394 | 18.229 | 1.00 | 0.57 | 1SG | 1701 |
| ATOM | 1701 | CB | PRO | 206 | −3.256 | 23.966 | 19.963 | 1.00 | 0.57 | 1SG | 1702 |
| ATOM | 1702 | CG | PRO | 206 | −2.487 | 25.281 | 19.755 | 1.00 | 0.57 | 1SG | 1703 |
| ATOM | 1703 | C | PRO | 206 | −5.534 | 24.174 | 18.943 | 1.00 | 0.57 | 1SG | 1704 |
| ATOM | 1704 | O | PRO | 206 | −5.929 | 25.287 | 18.600 | 1.00 | 0.57 | 1SG | 1705 |
| ATOM | 1705 | N | TRP | 207 | −6.334 | 23.252 | 19.514 | 1.00 | 0.68 | 1SG | 1706 |
| ATOM | 1706 | CA | TRP | 207 | −7.677 | 23.639 | 19.810 | 1.00 | 0.68 | 1SG | 1707 |
| ATOM | 1707 | CB | TRP | 207 | −8.692 | 23.244 | 18.721 | 1.00 | 0.68 | 1SG | 1708 |
| ATOM | 1708 | CG | TRP | 207 | −8.882 | 21.772 | 18.457 | 1.00 | 0.68 | 1SG | 1709 |
| ATOM | 1709 | CD2 | TRP | 207 | −9.925 | 20.979 | 19.043 | 1.00 | 0.68 | 1SG | 1710 |
| ATOM | 1710 | CD1 | TRP | 207 | −8.196 | 20.947 | 17.619 | 1.00 | 0.68 | 1SG | 1711 |
| ATOM | 1711 | NE1 | TRP | 207 | −8.738 | 19.685 | 17.654 | 1.00 | 0.68 | 1SG | 1712 |
| ATOM | 1712 | CE2 | TRP | 207 | −9.806 | 19.692 | 18.522 | 1.00 | 0.68 | 1SG | 1713 |
| ATOM | 1713 | CE3 | TRP | 207 | −10.909 | 21.303 | 19.929 | 1.00 | 0.68 | 1SG | 1714 |
| ATOM | 1714 | CZ2 | TRP | 207 | −10.674 | 18.702 | 18.884 | 1.00 | 0.68 | 1SG | 1715 |
| ATOM | 1715 | CZ3 | TRP | 207 | −11.776 | 20.300 | 20.300 | 1.00 | 0.68 | 1SG | 1716 |
| ATOM | 1716 | CH2 | TRP | 207 | −11.662 | 19.025 | 19.788 | 1.00 | 0.68 | 1SG | 1717 |
| ATOM | 1717 | C | TRP | 207 | −8.092 | 23.031 | 21.117 | 1.00 | 0.68 | 1SG | 1718 |
| ATOM | 1718 | O | TRP | 207 | −9.218 | 23.250 | 21.558 | 1.00 | 0.68 | 1SG | 1719 |
| ATOM | 1719 | N | ASP | 208 | −7.204 | 22.268 | 21.793 | 1.00 | 1.09 | 1SG | 1720 |
| ATOM | 1720 | CA | ASP | 208 | −7.615 | 21.711 | 23.056 | 1.00 | 1.09 | 1SG | 1721 |
| ATOM | 1721 | CB | ASP | 208 | −8.097 | 20.256 | 22.988 | 1.00 | 1.09 | 1SG | 1722 |
| ATOM | 1722 | CG | ASP | 208 | −9.501 | 20.197 | 22.424 | 1.00 | 1.09 | 1SG | 1723 |
| ATOM | 1723 | OD1 | ASP | 208 | −10.265 | 21.184 | 22.594 | 1.00 | 1.09 | 1SG | 1724 |
| ATOM | 1724 | OD2 | ASP | 208 | −9.828 | 19.141 | 21.825 | 1.00 | 1.09 | 1SG | 1725 |
| ATOM | 1725 | C | ASP | 208 | −6.462 | 21.659 | 24.009 | 1.00 | 1.09 | 1SG | 1726 |
| ATOM | 1726 | O | ASP | 208 | −5.497 | 20.937 | 23.766 | 1.00 | 1.09 | 1SG | 1727 |
| ATOM | 1727 | N | ARG | 209 | −6.539 | 22.424 | 25.122 | 1.00 | 1.08 | 1SG | 1728 |
| ATOM | 1728 | CA | ARG | 209 | −5.529 | 22.352 | 26.142 | 1.00 | 1.08 | 1SG | 1729 |
| ATOM | 1729 | CB | ARG | 209 | −4.236 | 23.107 | 25.777 | 1.00 | 1.08 | 1SG | 1730 |
| ATOM | 1730 | CG | ARG | 209 | −3.430 | 22.509 | 24.624 | 1.00 | 1.08 | 1SG | 1731 |
| ATOM | 1731 | CD | ARG | 209 | −2.666 | 21.238 | 25.000 | 1.00 | 1.08 | 1SG | 1732 |
| ATOM | 1732 | NE | ARG | 209 | −1.649 | 21.612 | 26.023 | 1.00 | 1.08 | 1SG | 1733 |
| ATOM | 1733 | CZ | ARG | 209 | −1.981 | 21.614 | 27.347 | 1.00 | 1.08 | 1SG | 1734 |
| ATOM | 1734 | NH1 | ARG | 209 | −3.242 | 21.263 | 27.736 | 1.00 | 1.08 | 1SG | 1735 |
| ATOM | 1735 | NH2 | ARG | 209 | −1.050 | 21.962 | 28.282 | 1.00 | 1.08 | 1SG | 1736 |
| ATOM | 1736 | C | ARG | 209 | −6.045 | 23.067 | 27.354 | 1.00 | 1.08 | 1SG | 1737 |
| ATOM | 1737 | O | ARG | 209 | −5.334 | 23.194 | 28.349 | 1.00 | 1.08 | 1SG | 1738 |
| ATOM | 1738 | N | TYR | 210 | −7.312 | 23.520 | 27.328 | 1.00 | 0.72 | 1SG | 1739 |
| ATOM | 1739 | CA | TYR | 210 | −7.780 | 24.330 | 28.417 | 1.00 | 0.72 | 1SG | 1740 |
| ATOM | 1740 | CB | TYR | 210 | −9.029 | 25.161 | 28.080 | 1.00 | 0.72 | 1SG | 1741 |
| ATOM | 1741 | CG | TYR | 210 | −8.606 | 26.184 | 27.084 | 1.00 | 0.72 | 1SG | 1742 |
| ATOM | 1742 | CD1 | TYR | 210 | −8.530 | 25.873 | 25.746 | 1.00 | 0.72 | 1SG | 1743 |
| ATOM | 1743 | CD2 | TYR | 210 | −8.280 | 27.455 | 27.494 | 1.00 | 0.72 | 1SG | 1744 |
| ATOM | 1744 | CE1 | TYR | 210 | −8.137 | 26.820 | 24.830 | 1.00 | 0.72 | 1SG | 1745 |
| ATOM | 1745 | CE2 | TYR | 210 | −7.885 | 28.406 | 26.583 | 1.00 | 0.72 | 1SG | 1746 |
| ATOM | 1746 | CZ | TYR | 210 | −7.814 | 28.088 | 25.248 | 1.00 | 0.72 | 1SG | 1747 |
| ATOM | 1747 | OH | TYR | 210 | −7.409 | 29.062 | 24.311 | 1.00 | 0.72 | 1SG | 1748 |
| ATOM | 1748 | C | TYR | 210 | −8.095 | 23.495 | 29.611 | 1.00 | 0.72 | 1SG | 1749 |
| ATOM | 1749 | O | TYR | 210 | −8.714 | 22.435 | 29.537 | 1.00 | 0.72 | 1SG | 1750 |
| ATOM | 1750 | N | PRO | 211 | −7.619 | 23.986 | 30.722 | 1.00 | 0.56 | 1SG | 1751 |
| ATOM | 1751 | CA | PRO | 211 | −7.904 | 23.356 | 31.976 | 1.00 | 0.56 | 1SG | 1752 |
| ATOM | 1752 | CD | PRO | 211 | −6.327 | 24.654 | 30.733 | 1.00 | 0.56 | 1SG | 1753 |
| ATOM | 1753 | CB | PRO | 211 | −6.904 | 23.928 | 32.977 | 1.00 | 0.56 | 1SG | 1754 |
| ATOM | 1754 | CG | PRO | 211 | −5.704 | 24.324 | 32.099 | 1.00 | 0.56 | 1SG | 1755 |
| ATOM | 1755 | C | PRO | 211 | −9.333 | 23.562 | 32.360 | 1.00 | 0.56 | 1SG | 1756 |
| ATOM | 1756 | O | PRO | 211 | −9.879 | 22.693 | 33.038 | 1.00 | 0.56 | 1SG | 1757 |
| ATOM | 1757 | N | PRO | 212 | −9.951 | 24.652 | 31.994 | 1.00 | 0.47 | 1SG | 1758 |
| ATOM | 1758 | CA | PRO | 212 | −11.335 | 24.783 | 32.336 | 1.00 | 0.47 | 1SG | 1759 |
| ATOM | 1759 | CD | PRO | 212 | −9.279 | 25.943 | 31.981 | 1.00 | 0.47 | 1SG | 1760 |
| ATOM | 1760 | CB | PRO | 212 | −11.695 | 26.240 | 32.060 | 1.00 | 0.47 | 1SG | 1761 |
| ATOM | 1761 | CG | PRO | 212 | −10.368 | 26.980 | 32.309 | 1.00 | 0.47 | 1SG | 1762 |
| ATOM | 1762 | C | PRO | 212 | −12.115 | 23.769 | 31.575 | 1.00 | 0.47 | 1SG | 1763 |
| ATOM | 1763 | O | PRO | 212 | −13.165 | 23.341 | 32.052 | 1.00 | 0.47 | 1SG | 1764 |
| ATOM | 1764 | N | PHE | 213 | −11.646 | 23.416 | 30.364 | 1.00 | 0.48 | 1SG | 1765 |
| ATOM | 1765 | CA | PHE | 213 | −12.296 | 22.398 | 29.598 | 1.00 | 0.48 | 1SG | 1766 |
| ATOM | 1766 | CB | PHE | 213 | −11.885 | 22.399 | 28.117 | 1.00 | 0.48 | 1SG | 1767 |
| ATOM | 1767 | CG | PHE | 213 | −12.544 | 23.589 | 27.508 | 1.00 | 0.48 | 1SG | 1768 |
| ATOM | 1768 | CD1 | PHE | 213 | −13.856 | 23.517 | 27.097 | 1.00 | 0.48 | 1SG | 1769 |
| ATOM | 1769 | CD2 | PHE | 213 | −11.862 | 24.774 | 27.349 | 1.00 | 0.48 | 1SG | 1770 |
| ATOM | 1770 | CE1 | PHE | 213 | −14.480 | 24.606 | 26.536 | 1.00 | 0.48 | 1SG | 1771 |
| ATOM | 1771 | CE2 | PHE | 213 | −12.481 | 25.866 | 26.788 | 1.00 | 0.48 | 1SG | 1772 |
| ATOM | 1772 | CZ | PHE | 213 | −13.791 | 25.784 | 26.380 | 1.00 | 0.48 | 1SG | 1773 |
| ATOM | 1773 | C | PHE | 213 | −11.994 | 21.055 | 30.186 | 1.00 | 0.48 | 1SG | 1774 |
| ATOM | 1774 | O | PHE | 213 | −12.887 | 20.222 | 30.325 | 1.00 | 0.48 | 1SG | 1775 |
| ATOM | 1775 | N | CYS | 214 | −10.719 | 20.809 | 30.559 | 1.00 | 0.54 | 1SG | 1776 |
| ATOM | 1776 | CA | CYS | 214 | −10.368 | 19.512 | 31.066 | 1.00 | 0.54 | 1SG | 1777 |
| ATOM | 1777 | CB | CYS | 214 | −8.856 | 19.308 | 31.285 | 1.00 | 0.54 | 1SG | 1778 |
| ATOM | 1778 | SG | CYS | 214 | −8.163 | 20.321 | 32.622 | 1.00 | 0.54 | 1SG | 1779 |
| ATOM | 1779 | C | CYS | 214 | −11.073 | 19.289 | 32.364 | 1.00 | 0.54 | 1SG | 1780 |
| ATOM | 1780 | O | CYS | 214 | −11.693 | 18.251 | 32.570 | 1.00 | 0.54 | 1SG | 1781 |
| ATOM | 1781 | N | SER | 215 | −11.040 | 20.278 | 33.269 | 1.00 | 0.41 | 1SG | 1782 |
| ATOM | 1782 | CA | SER | 215 | −11.714 | 20.132 | 34.526 | 1.00 | 0.41 | 1SG | 1783 |
| ATOM | 1783 | CB | SER | 215 | −13.217 | 19.832 | 34.357 | 1.00 | 0.41 | 1SG | 1784 |
| ATOM | 1784 | OG | SER | 215 | −13.858 | 19.742 | 35.622 | 1.00 | 0.41 | 1SG | 1785 |
| ATOM | 1785 | C | SER | 215 | −11.078 | 19.035 | 35.337 | 1.00 | 0.41 | 1SG | 1786 |
| ATOM | 1786 | O | SER | 215 | −11.740 | 18.373 | 36.135 | 1.00 | 0.41 | 1SG | 1787 |
| ATOM | 1787 | N | GLY | 216 | −9.766 | 18.796 | 35.148 | 1.00 | 0.40 | 1SG | 1788 |
| ATOM | 1788 | CA | GLY | 216 | −9.059 | 17.893 | 36.015 | 1.00 | 0.40 | 1SG | 1789 |
| ATOM | 1789 | C | GLY | 216 | −9.270 | 16.460 | 35.633 | 1.00 | 0.40 | 1SG | 1790 |
| ATOM | 1790 | O | GLY | 216 | −8.778 | 15.563 | 36.315 | 1.00 | 0.40 | 1SG | 1791 |
| ATOM | 1791 | N | THR | 217 | −10.004 | 16.184 | 34.543 | 1.00 | 0.60 | 1SG | 1792 |
| ATOM | 1792 | CA | THR | 217 | −10.189 | 14.812 | 34.164 | 1.00 | 0.60 | 1SG | 1793 |
| ATOM | 1793 | CB | THR | 217 | −11.394 | 14.608 | 33.282 | 1.00 | 0.60 | 1SG | 1794 |
| ATOM | 1794 | OG1 | THR | 217 | −11.469 | 13.268 | 32.823 | 1.00 | 0.60 | 1SG | 1795 |
| ATOM | 1795 | CG2 | THR | 217 | −11.365 | 15.592 | 32.111 | 1.00 | 0.60 | 1SG | 1796 |
| ATOM | 1796 | C | THR | 217 | −8.938 | 14.329 | 33.497 | 1.00 | 0.60 | 1SG | 1797 |
| ATOM | 1797 | O | THR | 217 | −8.077 | 15.128 | 33.129 | 1.00 | 0.60 | 1SG | 1798 |
| ATOM | 1798 | N | GLY | 218 | −8.795 | 12.990 | 33.331 | 1.00 | 0.81 | 1SG | 1799 |
| ATOM | 1799 | CA | GLY | 218 | −7.587 | 12.483 | 32.730 | 1.00 | 0.81 | 1SG | 1800 |
| ATOM | 1800 | C | GLY | 218 | −7.851 | 11.189 | 32.012 | 1.00 | 0.81 | 1SG | 1801 |
| ATOM | 1801 | O | GLY | 218 | −8.984 | 10.715 | 31.946 | 1.00 | 0.81 | 1SG | 1802 |
| ATOM | 1802 | N | TYR | 219 | −6.783 | 10.606 | 31.411 | 1.00 | 1.03 | 1SG | 1803 |
| ATOM | 1803 | CA | TYR | 219 | −6.912 | 9.356 | 30.713 | 1.00 | 1.03 | 1SG | 1804 |
| ATOM | 1804 | CB | TYR | 219 | −6.975 | 9.461 | 29.174 | 1.00 | 1.03 | 1SG | 1805 |
| ATOM | 1805 | CG | TYR | 219 | −7.641 | 8.200 | 28.718 | 1.00 | 1.03 | 1SG | 1806 |
| ATOM | 1806 | CD1 | TYR | 219 | −8.978 | 8.013 | 28.989 | 1.00 | 1.03 | 1SG | 1807 |
| ATOM | 1807 | CD2 | TYR | 219 | −6.973 | 7.224 | 28.013 | 1.00 | 1.03 | 1SG | 1808 |
| ATOM | 1808 | CE1 | TYR | 219 | −9.635 | 6.870 | 28.598 | 1.00 | 1.03 | 1SG | 1809 |
| ATOM | 1809 | CE2 | TYR | 219 | −7.624 | 6.077 | 27.618 | 1.00 | 1.03 | 1SG | 1810 |
| ATOM | 1810 | CZ | TYR | 219 | −8.956 | 5.896 | 27.910 | 1.00 | 1.03 | 1SG | 1811 |
| ATOM | 1811 | OH | TYR | 219 | −9.624 | 4.720 | 27.505 | 1.00 | 1.03 | 1SG | 1812 |
| ATOM | 1812 | C | TYR | 219 | −5.740 | 8.492 | 31.121 | 1.00 | 1.03 | 1SG | 1813 |
| ATOM | 1813 | O | TYR | 219 | −5.057 | 8.803 | 32.095 | 1.00 | 1.03 | 1SG | 1814 |
| ATOM | 1814 | N | VAL | 220 | −5.464 | 7.388 | 30.382 | 1.00 | 1.04 | 1SG | 1815 |
| ATOM | 1815 | CA | VAL | 220 | −4.508 | 6.392 | 30.818 | 1.00 | 1.04 | 1SG | 1816 |
| ATOM | 1816 | CB | VAL | 220 | −5.147 | 5.045 | 31.011 | 1.00 | 1.04 | 1SG | 1817 |
| ATOM | 1817 | CG1 | VAL | 220 | −6.244 | 5.185 | 32.085 | 1.00 | 1.04 | 1SG | 1818 |
| ATOM | 1818 | CG2 | VAL | 220 | −5.671 | 4.538 | 29.655 | 1.00 | 1.04 | 1SG | 1819 |
| ATOM | 1819 | C | VAL | 220 | −3.359 | 6.211 | 29.845 | 1.00 | 1.04 | 1SG | 1820 |
| ATOM | 1820 | O | VAL | 220 | −2.883 | 7.173 | 29.247 | 1.00 | 1.04 | 1SG | 1821 |
| ATOM | 1821 | N | PHE | 221 | −2.874 | 4.941 | 29.717 | 1.00 | 0.89 | 1SG | 1822 |
| ATOM | 1822 | CA | PHE | 221 | −1.784 | 4.420 | 28.912 | 1.00 | 0.89 | 1SG | 1823 |
| ATOM | 1823 | CB | PHE | 221 | −2.152 | 4.261 | 27.417 | 1.00 | 0.89 | 1SG | 1824 |
| ATOM | 1824 | CG | PHE | 221 | −2.505 | 5.627 | 26.946 | 1.00 | 0.89 | 1SG | 1825 |
| ATOM | 1825 | CD1 | PHE | 221 | −1.531 | 6.499 | 26.514 | 1.00 | 0.89 | 1SG | 1826 |
| ATOM | 1826 | CD2 | PHE | 221 | −3.817 | 6.037 | 26.952 | 1.00 | 0.89 | 1SG | 1827 |
| ATOM | 1827 | CE1 | PHE | 221 | −1.857 | 7.761 | 26.097 | 1.00 | 0.89 | 1SG | 1828 |
| ATOM | 1828 | CE2 | PHE | 221 | −4.140 | 7.303 | 26.538 | 1.00 | 0.89 | 1SG | 1829 |
| ATOM | 1829 | CZ | PHE | 221 | −3.162 | 8.173 | 26.124 | 1.00 | 0.89 | 1SG | 1830 |
| ATOM | 1830 | C | PHE | 221 | −0.575 | 5.288 | 29.000 | 1.00 | 0.89 | 1SG | 1831 |
| ATOM | 1831 | O | PHE | 221 | 0.336 | 5.171 | 28.184 | 1.00 | 0.89 | 1SG | 1832 |
| ATOM | 1832 | N | SER | 222 | −0.507 | 6.171 | 30.009 | 1.00 | 0.69 | 1SG | 1833 |
| ATOM | 1833 | CA | SER | 222 | 0.669 | 6.982 | 30.047 | 1.00 | 0.69 | 1SG | 1834 |
| ATOM | 1834 | CB | SER | 222 | 0.605 | 8.134 | 31.062 | 1.00 | 0.69 | 1SG | 1835 |
| ATOM | 1835 | OG | SER | 222 | 1.805 | 8.893 | 31.018 | 1.00 | 0.69 | 1SG | 1836 |
| ATOM | 1836 | C | SER | 222 | 1.802 | 6.087 | 30.425 | 1.00 | 0.69 | 1SG | 1837 |
| ATOM | 1837 | O | SER | 222 | 1.879 | 5.601 | 31.552 | 1.00 | 0.69 | 1SG | 1838 |
| ATOM | 1838 | N | GLY | 223 | 2.713 | 5.840 | 29.468 | 1.00 | 0.58 | 1SG | 1839 |
| ATOM | 1839 | CA | GLY | 223 | 3.831 | 5.010 | 29.785 | 1.00 | 0.58 | 1SG | 1840 |
| ATOM | 1840 | C | GLY | 223 | 3.731 | 3.733 | 29.017 | 1.00 | 0.58 | 1SG | 1841 |
| ATOM | 1841 | O | GLY | 223 | 4.732 | 3.037 | 28.856 | 1.00 | 0.58 | 1SG | 1842 |
| ATOM | 1842 | N | ASP | 224 | 2.537 | 3.375 | 28.504 | 1.00 | 0.57 | 1SG | 1843 |
| ATOM | 1843 | CA | ASP | 224 | 2.477 | 2.129 | 27.796 | 1.00 | 0.57 | 1SG | 1844 |
| ATOM | 1844 | CB | ASP | 224 | 1.057 | 1.665 | 27.414 | 1.00 | 0.57 | 1SG | 1845 |
| ATOM | 1845 | CG | ASP | 224 | 0.440 | 2.587 | 26.370 | 1.00 | 0.57 | 1SG | 1846 |
| ATOM | 1846 | OD1 | ASP | 224 | 0.941 | 3.727 | 26.185 | 1.00 | 0.57 | 1SG | 1847 |
| ATOM | 1847 | OD2 | ASP | 224 | −0.558 | 2.153 | 25.735 | 1.00 | 0.57 | 1SG | 1848 |
| ATOM | 1848 | C | ASP | 224 | 3.239 | 2.284 | 26.526 | 1.00 | 0.57 | 1SG | 1849 |
| ATOM | 1849 | O | ASP | 224 | 3.945 | 3.268 | 26.322 | 1.00 | 0.57 | 1SG | 1850 |
| ATOM | 1850 | N | VAL | 225 | 3.143 | 1.284 | 25.637 | 1.00 | 0.38 | 1SG | 1851 |
| ATOM | 1851 | CA | VAL | 225 | 3.751 | 1.457 | 24.358 | 1.00 | 0.38 | 1SG | 1852 |
| ATOM | 1852 | CB | VAL | 225 | 4.016 | 0.186 | 23.605 | 1.00 | 0.38 | 1SG | 1853 |
| ATOM | 1853 | CG1 | VAL | 225 | 4.495 | 0.574 | 22.195 | 1.00 | 0.38 | 1SG | 1854 |
| ATOM | 1854 | CG2 | VAL | 225 | 5.015 | −0.681 | 24.392 | 1.00 | 0.38 | 1SG | 1855 |
| ATOM | 1855 | C | VAL | 225 | 2.724 | 2.198 | 23.573 | 1.00 | 0.38 | 1SG | 1856 |
| ATOM | 1856 | O | VAL | 225 | 1.530 | 1.918 | 23.685 | 1.00 | 0.38 | 1SG | 1857 |
| ATOM | 1857 | N | ALA | 226 | 3.161 | 3.185 | 22.774 | 1.00 | 0.19 | 1SG | 1858 |
| ATOM | 1858 | CA | ALA | 226 | 2.214 | 3.969 | 22.043 | 1.00 | 0.19 | 1SG | 1859 |
| ATOM | 1859 | CB | ALA | 226 | 2.845 | 5.125 | 21.249 | 1.00 | 0.19 | 1SG | 1860 |
| ATOM | 1860 | C | ALA | 226 | 1.533 | 3.079 | 21.068 | 1.00 | 0.19 | 1SG | 1861 |
| ATOM | 1861 | O | ALA | 226 | 2.154 | 2.231 | 20.429 | 1.00 | 0.19 | 1SG | 1862 |
| ATOM | 1862 | N | SER | 227 | 0.203 | 3.224 | 20.977 | 1.00 | 0.25 | 1SG | 1863 |
| ATOM | 1863 | CA | SER | 227 | −0.583 | 2.459 | 20.066 | 1.00 | 0.25 | 1SG | 1864 |
| ATOM | 1864 | CB | SER | 227 | −2.071 | 2.480 | 20.440 | 1.00 | 0.25 | 1SG | 1865 |
| ATOM | 1865 | OG | SER | 227 | −2.691 | 3.654 | 19.945 | 1.00 | 0.25 | 1SG | 1866 |
| ATOM | 1866 | C | SER | 227 | −0.405 | 3.032 | 18.686 | 1.00 | 0.25 | 1SG | 1867 |
| ATOM | 1867 | O | SER | 227 | −0.545 | 2.325 | 17.689 | 1.00 | 0.25 | 1SG | 1868 |
| ATOM | 1868 | N | GLN | 228 | −0.103 | 4.347 | 18.589 | 1.00 | 0.40 | 1SG | 1869 |
| ATOM | 1869 | CA | GLN | 228 | −0.038 | 4.980 | 17.299 | 1.00 | 0.40 | 1SG | 1870 |
| ATOM | 1870 | CB | GLN | 228 | −1.067 | 6.118 | 17.149 | 1.00 | 0.40 | 1SG | 1871 |
| ATOM | 1871 | CG | GLN | 228 | −1.178 | 6.699 | 15.739 | 1.00 | 0.40 | 1SG | 1872 |
| ATOM | 1872 | CD | GLN | 228 | −2.233 | 7.796 | 15.789 | 1.00 | 0.40 | 1SG | 1873 |
| ATOM | 1873 | OE1 | GLN | 228 | −3.383 | 7.552 | 16.153 | 1.00 | 0.40 | 1SG | 1874 |
| ATOM | 1874 | NE2 | GLN | 228 | −1.837 | 9.043 | 15.424 | 1.00 | 0.40 | 1SG | 1875 |
| ATOM | 1875 | C | GLN | 228 | 1.332 | 5.538 | 17.049 | 1.00 | 0.40 | 1SG | 1876 |
| ATOM | 1876 | O | GLN | 228 | 2.190 | 5.564 | 17.929 | 1.00 | 0.40 | 1SG | 1877 |
| ATOM | 1877 | N | VAL | 229 | 1.549 | 5.989 | 15.783 | 1.00 | 0.64 | 1SG | 1878 |
| ATOM | 1878 | CA | VAL | 229 | 2.804 | 6.506 | 15.297 | 1.00 | 0.64 | 1SG | 1879 |
| ATOM | 1879 | CB | VAL | 229 | 3.249 | 5.763 | 14.065 | 1.00 | 0.64 | 1SG | 1880 |
| ATOM | 1880 | CG1 | VAL | 229 | 4.585 | 6.325 | 13.538 | 1.00 | 0.64 | 1SG | 1881 |
| ATOM | 1881 | CG2 | VAL | 229 | 3.300 | 4.271 | 14.414 | 1.00 | 0.64 | 1SG | 1882 |
| ATOM | 1882 | C | VAL | 229 | 2.598 | 7.969 | 14.948 | 1.00 | 0.64 | 1SG | 1883 |
| ATOM | 1883 | O | VAL | 229 | 1.468 | 8.436 | 14.837 | 1.00 | 0.64 | 1SG | 1884 |
| ATOM | 1884 | N | TYR | 230 | 3.717 | 8.703 | 14.765 | 1.00 | 0.90 | 1SG | 1885 |
| ATOM | 1885 | CA | TYR | 230 | 3.820 | 10.133 | 14.577 | 1.00 | 0.90 | 1SG | 1886 |
| ATOM | 1886 | CB | TYR | 230 | 5.218 | 10.589 | 14.124 | 1.00 | 0.90 | 1SG | 1887 |
| ATOM | 1887 | CG | TYR | 230 | 5.413 | 10.102 | 12.729 | 1.00 | 0.90 | 1SG | 1888 |
| ATOM | 1888 | CD1 | TYR | 230 | 5.783 | 8.800 | 12.479 | 1.00 | 0.90 | 1SG | 1889 |
| ATOM | 1889 | CD2 | TYR | 230 | 5.225 | 10.957 | 11.667 | 1.00 | 0.90 | 1SG | 1890 |
| ATOM | 1890 | CE1 | TYR | 230 | 5.962 | 8.356 | 11.190 | 1.00 | 0.90 | 1SG | 1891 |
| ATOM | 1891 | CE2 | TYR | 230 | 5.404 | 10.518 | 10.377 | 1.00 | 0.90 | 1SG | 1892 |
| ATOM | 1892 | CZ | TYR | 230 | 5.772 | 9.217 | 10.137 | 1.00 | 0.90 | 1SG | 1893 |
| ATOM | 1893 | OH | TYR | 230 | 5.956 | 8.766 | 8.813 | 1.00 | 0.90 | 1SG | 1894 |
| ATOM | 1894 | C | TYR | 230 | 2.857 | 10.685 | 13.578 | 1.00 | 0.90 | 1SG | 1895 |
| ATOM | 1895 | O | TYR | 230 | 2.184 | 9.960 | 12.847 | 1.00 | 0.90 | 1SG | 1896 |
| ATOM | 1896 | N | ASN | 231 | 2.781 | 12.037 | 13.565 | 1.00 | 1.04 | 1SG | 1897 |
| ATOM | 1897 | CA | ASN | 231 | 1.960 | 12.836 | 12.701 | 1.00 | 1.04 | 1SG | 1898 |
| ATOM | 1898 | CB | ASN | 231 | 0.573 | 13.143 | 13.285 | 1.00 | 1.04 | 1SG | 1899 |
| ATOM | 1899 | CG | ASN | 231 | −0.212 | 11.843 | 13.349 | 1.00 | 1.04 | 1SG | 1900 |
| ATOM | 1900 | OD1 | ASN | 231 | −0.360 | 11.141 | 12.349 | 1.00 | 1.04 | 1SG | 1901 |
| ATOM | 1901 | ND2 | ASN | 231 | −0.725 | 11.508 | 14.563 | 1.00 | 1.04 | 1SG | 1902 |
| ATOM | 1902 | C | ASN | 231 | 2.663 | 14.153 | 12.566 | 1.00 | 1.04 | 1SG | 1903 |
| ATOM | 1903 | O | ASN | 231 | 3.883 | 14.226 | 12.709 | 1.00 | 1.04 | 1SG | 1904 |
| ATOM | 1904 | N | VAL | 232 | 1.912 | 15.236 | 12.270 | 1.00 | 1.06 | 1SG | 1905 |
| ATOM | 1905 | CA | VAL | 232 | 2.550 | 16.517 | 12.145 | 1.00 | 1.06 | 1SG | 1906 |
| ATOM | 1906 | CB | VAL | 232 | 2.067 | 17.344 | 10.991 | 1.00 | 1.06 | 1SG | 1907 |
| ATOM | 1907 | CG1 | VAL | 232 | 2.345 | 16.580 | 9.687 | 1.00 | 1.06 | 1SG | 1908 |
| ATOM | 1908 | CG2 | VAL | 232 | 0.593 | 17.714 | 11.228 | 1.00 | 1.06 | 1SG | 1909 |
| ATOM | 1909 | C | VAL | 232 | 2.245 | 17.307 | 13.375 | 1.00 | 1.06 | 1SG | 1910 |
| ATOM | 1910 | O | VAL | 232 | 1.130 | 17.268 | 13.892 | 1.00 | 1.06 | 1SG | 1911 |
| ATOM | 1911 | N | SER | 233 | 3.261 | 18.029 | 13.882 | 1.00 | 1.12 | 1SG | 1912 |
| ATOM | 1912 | CA | SER | 233 | 3.115 | 18.867 | 15.035 | 1.00 | 1.12 | 1SG | 1913 |
| ATOM | 1913 | CB | SER | 233 | 2.296 | 18.238 | 16.177 | 1.00 | 1.12 | 1SG | 1914 |
| ATOM | 1914 | OG | SER | 233 | 2.950 | 17.081 | 16.679 | 1.00 | 1.12 | 1SG | 1915 |
| ATOM | 1915 | C | SER | 233 | 4.489 | 19.099 | 15.556 | 1.00 | 1.12 | 1SG | 1916 |
| ATOM | 1916 | O | SER | 233 | 5.473 | 18.697 | 14.937 | 1.00 | 1.12 | 1SG | 1917 |
| ATOM | 1917 | N | LYS | 234 | 4.596 | 19.782 | 16.710 | 1.00 | 1.27 | 1SG | 1918 |
| ATOM | 1918 | CA | LYS | 234 | 5.900 | 19.973 | 17.264 | 1.00 | 1.27 | 1SG | 1919 |
| ATOM | 1919 | CB | LYS | 234 | 6.091 | 21.328 | 17.964 | 1.00 | 1.27 | 1SG | 1920 |
| ATOM | 1920 | CG | LYS | 234 | 5.999 | 22.522 | 17.012 | 1.00 | 1.27 | 1SG | 1921 |
| ATOM | 1921 | CD | LYS | 234 | 6.989 | 22.448 | 15.847 | 1.00 | 1.27 | 1SG | 1922 |
| ATOM | 1922 | CE | LYS | 234 | 8.447 | 22.306 | 16.283 | 1.00 | 1.27 | 1SG | 1923 |
| ATOM | 1923 | NZ | LYS | 234 | 8.878 | 23.520 | 17.011 | 1.00 | 1.27 | 1SG | 1924 |
| ATOM | 1924 | C | LYS | 234 | 6.076 | 18.911 | 18.296 | 1.00 | 1.27 | 1SG | 1925 |
| ATOM | 1925 | O | LYS | 234 | 5.815 | 19.124 | 19.478 | 1.00 | 1.27 | 1SG | 1926 |
| ATOM | 1926 | N | SER | 235 | 6.520 | 17.719 | 17.866 | 1.00 | 1.18 | 1SG | 1927 |
| ATOM | 1927 | CA | SER | 235 | 6.701 | 16.649 | 18.799 | 1.00 | 1.18 | 1SG | 1928 |
| ATOM | 1928 | CB | SER | 235 | 5.736 | 15.472 | 18.553 | 1.00 | 1.18 | 1SG | 1929 |
| ATOM | 1929 | OG | SER | 235 | 5.949 | 14.433 | 19.499 | 1.00 | 1.18 | 1SG | 1930 |
| ATOM | 1930 | C | SER | 235 | 8.093 | 16.157 | 18.619 | 1.00 | 1.18 | 1SG | 1931 |
| ATOM | 1931 | O | SER | 235 | 8.833 | 16.648 | 17.768 | 1.00 | 1.18 | 1SG | 1932 |
| ATOM | 1932 | N | VAL | 236 | 8.501 | 15.182 | 19.448 | 1.00 | 1.13 | 1SG | 1933 |
| ATOM | 1933 | CA | VAL | 236 | 9.813 | 14.646 | 19.289 | 1.00 | 1.13 | 1SG | 1934 |
| ATOM | 1934 | CB | VAL | 236 | 10.178 | 13.648 | 20.346 | 1.00 | 1.13 | 1SG | 1935 |
| ATOM | 1935 | CG1 | VAL | 236 | 10.231 | 14.374 | 21.702 | 1.00 | 1.13 | 1SG | 1936 |
| ATOM | 1936 | CG2 | VAL | 236 | 9.157 | 12.500 | 20.303 | 1.00 | 1.13 | 1SG | 1937 |
| ATOM | 1937 | C | VAL | 236 | 9.822 | 13.973 | 17.959 | 1.00 | 1.13 | 1SG | 1938 |
| ATOM | 1938 | O | VAL | 236 | 8.778 | 13.771 | 17.338 | 1.00 | 1.13 | 1SG | 1939 |
| ATOM | 1939 | N | PRO | 237 | 10.987 | 13.649 | 17.490 | 1.00 | 1.27 | 1SG | 1940 |
| ATOM | 1940 | CA | PRO | 237 | 11.073 | 13.048 | 16.191 | 1.00 | 1.27 | 1SG | 1941 |
| ATOM | 1941 | CD | PRO | 237 | 12.147 | 14.475 | 17.784 | 1.00 | 1.27 | 1SG | 1942 |
| ATOM | 1942 | CB | PRO | 237 | 12.537 | 13.163 | 15.778 | 1.00 | 1.27 | 1SG | 1943 |
| ATOM | 1943 | CG | PRO | 237 | 13.020 | 14.422 | 16.521 | 1.00 | 1.27 | 1SG | 1944 |
| ATOM | 1944 | C | PRO | 237 | 10.524 | 11.659 | 16.132 | 1.00 | 1.27 | 1SG | 1945 |
| ATOM | 1945 | O | PRO | 237 | 10.136 | 11.231 | 15.045 | 1.00 | 1.27 | 1SG | 1946 |
| ATOM | 1946 | N | TYR | 238 | 10.490 | 10.925 | 17.260 | 1.00 | 1.25 | 1SG | 1947 |
| ATOM | 1947 | CA | TYR | 238 | 9.978 | 9.584 | 17.193 | 1.00 | 1.25 | 1SG | 1948 |
| ATOM | 1948 | CB | TYR | 238 | 11.052 | 8.501 | 16.976 | 1.00 | 1.25 | 1SG | 1949 |
| ATOM | 1949 | CG | TYR | 238 | 11.605 | 8.589 | 15.597 | 1.00 | 1.25 | 1SG | 1950 |
| ATOM | 1950 | CD1 | TYR | 238 | 10.924 | 8.027 | 14.541 | 1.00 | 1.25 | 1SG | 1951 |
| ATOM | 1951 | CD2 | TYR | 238 | 12.807 | 9.212 | 15.359 | 1.00 | 1.25 | 1SG | 1952 |
| ATOM | 1952 | CE1 | TYR | 238 | 11.428 | 8.096 | 13.264 | 1.00 | 1.25 | 1SG | 1953 |
| ATOM | 1953 | CE2 | TYR | 238 | 13.317 | 9.284 | 14.084 | 1.00 | 1.25 | 1SG | 1954 |
| ATOM | 1954 | CZ | TYR | 238 | 12.627 | 8.727 | 13.034 | 1.00 | 1.25 | 1SG | 1955 |
| ATOM | 1955 | OH | TYR | 238 | 13.149 | 8.800 | 11.724 | 1.00 | 1.25 | 1SG | 1956 |
| ATOM | 1956 | C | TYR | 238 | 9.343 | 9.229 | 18.509 | 1.00 | 1.25 | 1SG | 1957 |
| ATOM | 1957 | O | TYR | 238 | 9.547 | 9.898 | 19.521 | 1.00 | 1.25 | 1SG | 1958 |
| ATOM | 1958 | N | ILE | 239 | 8.533 | 8.146 | 18.497 | 1.00 | 0.99 | 1SG | 1959 |
| ATOM | 1959 | CA | ILE | 239 | 7.857 | 7.586 | 19.637 | 1.00 | 0.99 | 1SG | 1960 |
| ATOM | 1960 | CB | ILE | 239 | 6.487 | 8.185 | 19.822 | 1.00 | 0.99 | 1SG | 1961 |
| ATOM | 1961 | CG2 | ILE | 239 | 5.630 | 7.717 | 18.634 | 1.00 | 0.99 | 1SG | 1962 |
| ATOM | 1962 | CG1 | ILE | 239 | 5.878 | 7.870 | 21.204 | 1.00 | 0.99 | 1SG | 1963 |
| ATOM | 1963 | CD1 | ILE | 239 | 5.496 | 6.407 | 21.418 | 1.00 | 0.99 | 1SG | 1964 |
| ATOM | 1964 | C | ILE | 239 | 7.707 | 6.139 | 19.274 | 1.00 | 0.99 | 1SG | 1965 |
| ATOM | 1965 | O | ILE | 239 | 7.548 | 5.818 | 18.097 | 1.00 | 0.99 | 1SG | 1966 |
| ATOM | 1966 | N | LYS | 240 | 7.770 | 5.212 | 20.249 | 1.00 | 0.53 | 1SG | 1967 |
| ATOM | 1967 | CA | LYS | 240 | 7.679 | 3.833 | 19.855 | 1.00 | 0.53 | 1SG | 1968 |
| ATOM | 1968 | CB | LYS | 240 | 8.285 | 2.822 | 20.839 | 1.00 | 0.53 | 1SG | 1969 |
| ATOM | 1969 | CG | LYS | 240 | 9.773 | 2.568 | 20.606 | 1.00 | 0.53 | 1SG | 1970 |
| ATOM | 1970 | CD | LYS | 240 | 10.387 | 1.586 | 21.605 | 1.00 | 0.53 | 1SG | 1971 |
| ATOM | 1971 | CE | LYS | 240 | 11.729 | 1.023 | 21.135 | 1.00 | 0.53 | 1SG | 1972 |
| ATOM | 1972 | NZ | LYS | 240 | 12.260 | 0.071 | 22.135 | 1.00 | 0.53 | 1SG | 1973 |
| ATOM | 1973 | C | LYS | 240 | 6.266 | 3.430 | 19.623 | 1.00 | 0.53 | 1SG | 1974 |
| ATOM | 1974 | O | LYS | 240 | 5.338 | 3.925 | 20.259 | 1.00 | 0.53 | 1SG | 1975 |
| ATOM | 1975 | N | LEU | 241 | 6.098 | 2.489 | 18.670 | 1.00 | 0.31 | 1SG | 1976 |
| ATOM | 1976 | CA | LEU | 241 | 4.822 | 1.952 | 18.308 | 1.00 | 0.31 | 1SG | 1977 |
| ATOM | 1977 | CB | LEU | 241 | 4.636 | 1.835 | 16.785 | 1.00 | 0.31 | 1SG | 1978 |
| ATOM | 1978 | CG | LEU | 241 | 3.275 | 1.258 | 16.358 | 1.00 | 0.31 | 1SG | 1979 |
| ATOM | 1979 | CD2 | LEU | 241 | 3.248 | 0.966 | 14.847 | 1.00 | 0.31 | 1SG | 1980 |
| ATOM | 1980 | CD1 | LEU | 241 | 2.118 | 2.166 | 16.810 | 1.00 | 0.31 | 1SG | 1981 |
| ATOM | 1981 | C | LEU | 241 | 4.776 | 0.574 | 18.886 | 1.00 | 0.31 | 1SG | 1982 |
| ATOM | 1982 | O | LEU | 241 | 5.777 | −0.141 | 18.896 | 1.00 | 0.31 | 1SG | 1983 |
| ATOM | 1983 | N | GLU | 242 | 3.600 | 0.166 | 19.399 | 1.00 | 0.43 | 1SG | 1984 |
| ATOM | 1984 | CA | GLU | 242 | 3.506 | −1.129 | 20.003 | 1.00 | 0.43 | 1SG | 1985 |
| ATOM | 1985 | CB | GLU | 242 | 2.214 | −1.325 | 20.816 | 1.00 | 0.43 | 1SG | 1986 |
| ATOM | 1986 | CG | GLU | 242 | 0.926 | −1.077 | 20.027 | 1.00 | 0.43 | 1SG | 1987 |
| ATOM | 1987 | CD | GLU | 242 | −0.248 | −1.240 | 20.983 | 1.00 | 0.43 | 1SG | 1988 |
| ATOM | 1988 | OE1 | GLU | 242 | −0.068 | −1.911 | 22.032 | 1.00 | 0.43 | 1SG | 1989 |
| ATOM | 1989 | OE2 | GLU | 242 | −1.343 | −0.697 | 20.688 | 1.00 | 0.43 | 1SG | 1990 |
| ATOM | 1990 | C | GLU | 242 | 3.593 | −2.172 | 18.938 | 1.00 | 0.43 | 1SG | 1991 |
| ATOM | 1991 | O | GLU | 242 | 2.625 | −2.471 | 18.238 | 1.00 | 0.43 | 1SG | 1992 |
| ATOM | 1992 | N | ASP | 243 | 4.817 | −2.698 | 18.770 | 1.00 | 0.56 | 1SG | 1993 |
| ATOM | 1993 | CA | ASP | 243 | 5.146 | −3.745 | 17.854 | 1.00 | 0.56 | 1SG | 1994 |
| ATOM | 1994 | CB | ASP | 243 | 6.670 | −3.902 | 17.738 | 1.00 | 0.56 | 1SG | 1995 |
| ATOM | 1995 | CG | ASP | 243 | 6.971 | −4.942 | 16.677 | 1.00 | 0.56 | 1SG | 1996 |
| ATOM | 1996 | OD1 | ASP | 243 | 6.003 | −5.362 | 15.986 | 1.00 | 0.56 | 1SG | 1997 |
| ATOM | 1957 | OD2 | ASP | 243 | 8.163 | −5.332 | 16.538 | 1.00 | 0.56 | 1SG | 1998 |
| ATOM | 1998 | C | ASP | 243 | 4.609 | −5.039 | 18.380 | 1.00 | 0.56 | 1SG | 1999 |
| ATOM | 1999 | O | ASP | 243 | 4.073 | −5.859 | 17.637 | 1.00 | 0.56 | 1SG | 2000 |
| ATOM | 2000 | N | VAL | 244 | 4.732 | −5.236 | 19.705 | 1.00 | 0.59 | 1SG | 2001 |
| ATOM | 2001 | CA | VAL | 244 | 4.410 | −6.491 | 20.324 | 1.00 | 0.59 | 1SG | 2002 |
| ATOM | 2002 | CB | VAL | 244 | 5.166 | −6.691 | 21.612 | 1.00 | 0.59 | 1SG | 2003 |
| ATOM | 2003 | CG1 | VAL | 244 | 4.744 | −5.581 | 22.592 | 1.00 | 0.59 | 1SG | 2004 |
| ATOM | 2004 | CG2 | VAL | 244 | 4.911 | −8.111 | 22.148 | 1.00 | 0.59 | 1SG | 2005 |
| ATOM | 2005 | C | VAL | 244 | 2.948 | −6.580 | 20.617 | 1.00 | 0.59 | 1SG | 2006 |
| ATOM | 2006 | O | VAL | 244 | 2.264 | −5.563 | 20.726 | 1.00 | 0.59 | 1SG | 2007 |
| ATOM | 2007 | N | PHE | 245 | 2.480 | −7.844 | 20.736 | 1.00 | 0.66 | 1SG | 2008 |
| ATOM | 2008 | CA | PHE | 245 | 1.163 | −8.327 | 21.044 | 1.00 | 0.66 | 1SG | 2009 |
| ATOM | 2009 | CB | PHE | 245 | 1.286 | −9.613 | 21.890 | 1.00 | 0.66 | 1SG | 2010 |
| ATOM | 2010 | CG | PHE | 245 | 0.002 | −10.371 | 21.952 | 1.00 | 0.66 | 1SG | 2011 |
| ATOM | 2011 | CD1 | PHE | 245 | −0.303 | −11.309 | 20.994 | 1.00 | 0.66 | 1SG | 2012 |
| ATOM | 2012 | CD2 | PHE | 245 | −0.897 | −10.157 | 22.972 | 1.00 | 0.66 | 1SG | 2013 |
| ATOM | 2013 | CE1 | PHE | 245 | −1.482 | −12.022 | 21.046 | 1.00 | 0.66 | 1SG | 2014 |
| ATOM | 2014 | CE2 | PHE | 245 | −2.077 | −10.864 | 23.029 | 1.00 | 0.66 | 1SG | 2015 |
| ATOM | 2015 | CZ | PHE | 245 | −2.372 | −11.799 | 22.068 | 1.00 | 0.66 | 1SG | 2016 |
| ATOM | 2016 | C | PHE | 245 | 0.465 | −7.295 | 21.862 | 1.00 | 0.66 | 1SG | 2017 |
| ATOM | 2017 | O | PHE | 245 | −0.054 | −6.315 | 21.351 | 1.00 | 0.66 | 1SG | 2018 |
| ATOM | 2018 | N | VAL | 246 | 0.428 | −7.475 | 23.183 | 1.00 | 0.80 | 1SG | 2019 |
| ATOM | 2019 | CA | VAL | 246 | −0.260 | −6.518 | 23.988 | 1.00 | 0.80 | 1SG | 2020 |
| ATOM | 2020 | CB | VAL | 246 | −1.692 | −6.277 | 23.557 | 1.00 | 0.80 | 1SG | 2021 |
| ATOM | 2021 | CG1 | VAL | 246 | −2.464 | −7.608 | 23.384 | 1.00 | 0.80 | 1SG | 2022 |
| ATOM | 2022 | CG2 | VAL | 246 | −2.317 | −5.290 | 24.563 | 1.00 | 0.80 | 1SG | 2023 |
| ATOM | 2023 | C | VAL | 246 | −0.158 | −7.029 | 25.381 | 1.00 | 0.80 | 1SG | 2024 |
| ATOM | 2024 | O | VAL | 246 | 0.880 | −6.894 | 26.026 | 1.00 | 0.80 | 1SG | 2025 |
| ATOM | 2025 | N | GLY | 247 | −1.249 | −7.591 | 25.906 | 1.00 | 0.83 | 1SG | 2026 |
| ATOM | 2026 | CA | GLY | 247 | −1.230 | −8.217 | 27.183 | 1.00 | 0.83 | 1SG | 2027 |
| ATOM | 2027 | C | GLY | 247 | −2.418 | −9.088 | 27.110 | 1.00 | 0.83 | 1SG | 2028 |
| ATOM | 2028 | O | GLY | 247 | −3.167 | −9.197 | 28.079 | 1.00 | 0.83 | 1SG | 2029 |
| ATOM | 2029 | N | LEU | 248 | −2.550 | −9.790 | 25.955 | 1.00 | 0.72 | 1SG | 2030 |
| ATOM | 2030 | CA | LEU | 248 | −3.691 | −10.543 | 25.495 | 1.00 | 0.72 | 1SG | 2031 |
| ATOM | 2031 | CB | LEU | 248 | −3.803 | −12.024 | 25.954 | 1.00 | 0.72 | 1SG | 2032 |
| ATOM | 2032 | CG | LEU | 248 | −3.676 | −12.324 | 27.462 | 1.00 | 0.72 | 1SG | 2033 |
| ATOM | 2033 | CD2 | LEU | 248 | −2.280 | −11.921 | 27.968 | 1.00 | 0.72 | 1SG | 2034 |
| ATOM | 2034 | CD1 | LEU | 248 | −4.008 | −13.804 | 27.773 | 1.00 | 0.72 | 1SG | 2035 |
| ATOM | 2035 | C | LEU | 248 | −4.972 | −9.833 | 25.768 | 1.00 | 0.72 | 1SG | 2036 |
| ATOM | 2036 | O | LEU | 248 | −4.999 | −8.781 | 26.408 | 1.00 | 0.72 | 1SG | 2037 |
| ATOM | 2037 | N | CYS | 249 | −6.040 | −10.374 | 25.129 | 1.00 | 0.42 | 1SG | 2038 |
| ATOM | 2038 | CA | CYS | 249 | −7.394 | −9.892 | 25.098 | 1.00 | 0.42 | 1SG | 2039 |
| ATOM | 2039 | CB | CYS | 249 | −8.437 | −11.017 | 25.079 | 1.00 | 0.42 | 1SG | 2040 |
| ATOM | 2040 | SG | CYS | 249 | −8.276 | −12.057 | 23.598 | 1.00 | 0.42 | 1SG | 2041 |
| ATOM | 2041 | C | CYS | 249 | −7.636 | −9.035 | 26.285 | 1.00 | 0.42 | 1SG | 2042 |
| ATOM | 2042 | O | CYS | 249 | −7.946 | −9.504 | 27.378 | 1.00 | 0.42 | 1SG | 2043 |
| ATOM | 2043 | N | LEU | 250 | −7.373 | −7.730 | 26.089 | 1.00 | 0.27 | 1SG | 2044 |
| ATOM | 2044 | CA | LEU | 250 | −7.515 | −6.751 | 27.115 | 1.00 | 0.27 | 1SG | 2045 |
| ATOM | 2045 | CB | LEU | 250 | −7.179 | −5.340 | 26.601 | 1.00 | 0.27 | 1SG | 2046 |
| ATOM | 2046 | CG | LEU | 250 | −5.732 | −5.229 | 26.085 | 1.00 | 0.27 | 1SG | 2047 |
| ATOM | 2047 | CD2 | LEU | 250 | −4.731 | −5.860 | 27.067 | 1.00 | 0.27 | 1SG | 2048 |
| ATOM | 2048 | CD1 | LEU | 250 | −5.390 | −3.778 | 25.724 | 1.00 | 0.27 | 1SG | 2049 |
| ATOM | 2049 | C | LEU | 250 | −8.954 | −6.821 | 27.467 | 1.00 | 0.27 | 1SG | 2050 |
| ATOM | 2050 | O | LEU | 250 | −9.345 | −6.703 | 28.628 | 1.00 | 0.27 | 1SG | 2051 |
| ATOM | 2051 | N | GLU | 251 | −9.771 | −7.056 | 26.430 | 1.00 | 0.40 | 1SG | 2052 |
| ATOM | 2052 | CA | GLU | 251 | −11.181 | −7.211 | 26.574 | 1.00 | 0.40 | 1SG | 2053 |
| ATOM | 2053 | CB | GLU | 251 | −11.849 | −7.547 | 25.232 | 1.00 | 0.40 | 1SG | 2054 |
| ATOM | 2054 | CG | GLU | 251 | −13.367 | −7.701 | 25.305 | 1.00 | 0.40 | 1SG | 2055 |
| ATOM | 2055 | CD | GLU | 251 | −13.823 | −8.096 | 23.910 | 1.00 | 0.40 | 1SG | 2056 |
| ATOM | 2056 | OE1 | GLU | 251 | −13.140 | −8.965 | 23.303 | 1.00 | 0.40 | 1SG | 2057 |
| ATOM | 2057 | OE2 | GLU | 251 | −14.845 | −7.536 | 23.430 | 1.00 | 0.40 | 1SG | 2058 |
| ATOM | 2058 | C | GLU | 251 | −11.377 | −8.384 | 27.478 | 1.00 | 0.40 | 1SG | 2059 |
| ATOM | 2059 | O | GLU | 251 | −12.255 | −8.382 | 28.337 | 1.00 | 0.40 | 1SG | 2060 |
| ATOM | 2060 | N | ARG | 252 | −10.542 | −9.424 | 27.309 | 1.00 | 0.44 | 1SG | 2061 |
| ATOM | 2061 | CA | ARG | 252 | −10.637 | −10.624 | 28.090 | 1.00 | 0.44 | 1SG | 2062 |
| ATOM | 2062 | CB | ARG | 252 | −9.673 | −11.743 | 27.658 | 1.00 | 0.44 | 1SG | 2063 |
| ATOM | 2063 | CG | ARG | 252 | −9.969 | −13.039 | 28.416 | 1.00 | 0.44 | 1SG | 2064 |
| ATOM | 2064 | CD | ARG | 252 | −9.068 | −14.227 | 23.083 | 1.00 | 0.44 | 1SG | 2065 |
| ATOM | 2065 | NE | ARG | 252 | −9.531 | −15.347 | 28.951 | 1.00 | 0.44 | 1SG | 2066 |
| ATOM | 2066 | CZ | ARG | 252 | −8.790 | −16.483 | 29.113 | 1.00 | 0.44 | 1SG | 2067 |
| ATOM | 2067 | NH1 | ARG | 252 | −7.585 | −16.622 | 28.486 | 1.00 | 0.44 | 1SG | 2068 |
| ATOM | 2068 | NH2 | ARG | 252 | −9.262 | −17.481 | 29.915 | 1.00 | 0.44 | 1SG | 2069 |
| ATOM | 2069 | C | ARG | 252 | −10.355 | −10.370 | 29.543 | 1.00 | 0.44 | 1SG | 2070 |
| ATOM | 2070 | O | ARG | 252 | −11.029 | −10.934 | 30.404 | 1.00 | 0.44 | 1SG | 2071 |
| ATOM | 2071 | N | LEU | 253 | −9.362 | −9.519 | 29.872 | 1.00 | 0.35 | 1SG | 2072 |
| ATOM | 2072 | CA | LEU | 253 | −9.052 | −9.354 | 31.268 | 1.00 | 0.35 | 1SG | 2073 |
| ATOM | 2073 | CB | LEU | 253 | −7.595 | −8.962 | 31.571 | 1.00 | 0.35 | 1SG | 2074 |
| ATOM | 2074 | CG | LEU | 253 | −6.574 | −10.064 | 31.236 | 1.00 | 0.35 | 1SG | 2075 |
| ATOM | 2075 | CD2 | LEU | 253 | −7.047 | −11.446 | 31.719 | 1.00 | 0.35 | 1SG | 2076 |
| ATOM | 2076 | CD1 | LEU | 253 | −5.179 | −9.694 | 31.762 | 1.00 | 0.35 | 1SG | 2077 |
| ATOM | 2077 | C | LEU | 253 | −9.932 | −8.309 | 31.874 | 1.00 | 0.35 | 1SG | 2078 |
| ATOM | 2078 | O | LEU | 253 | −10.201 | −7.269 | 31.273 | 1.00 | 0.35 | 1SG | 2079 |
| ATOM | 2079 | N | ASN | 254 | −10.415 | −8.588 | 33.102 | 1.00 | 0.53 | 1SG | 2080 |
| ATOM | 2080 | CA | ASN | 254 | −11.278 | −7.691 | 33.815 | 1.00 | 0.53 | 1SG | 2081 |
| ATOM | 2081 | CB | ASN | 254 | −12.371 | −8.438 | 34.601 | 1.00 | 0.53 | 1SG | 2082 |
| ATOM | 2082 | CG | ASN | 254 | −13.228 | −7.402 | 35.307 | 1.00 | 0.53 | 1SG | 2083 |
| ATOM | 2083 | OD1 | ASN | 254 | −13.531 | −6.345 | 34.758 | 1.00 | 0.53 | 1SG | 2084 |
| ATOM | 2084 | ND2 | ASN | 254 | −13.615 | −7.710 | 36.575 | 1.00 | 0.53 | 1SG | 2085 |
| ATOM | 2085 | C | ASN | 254 | −10.467 | −6.950 | 34.832 | 1.00 | 0.53 | 1SG | 2086 |
| ATOM | 2086 | O | ASN | 254 | −10.598 | −5.736 | 34.991 | 1.00 | 0.53 | 1SG | 2087 |
| ATOM | 2087 | N | ILE | 255 | −9.575 | −7.678 | 35.535 | 1.00 | 0.84 | 1SG | 2088 |
| ATOM | 2088 | CA | ILE | 255 | −8.825 | −7.122 | 36.623 | 1.00 | 0.84 | 1SG | 2089 |
| ATOM | 2089 | CB | ILE | 255 | −7.789 | −8.060 | 37.178 | 1.00 | 0.84 | 1SG | 2090 |
| ATOM | 2090 | CG2 | ILE | 255 | −8.514 | −9.317 | 37.685 | 1.00 | 0.84 | 1SG | 2091 |
| ATOM | 2091 | CG1 | ILE | 255 | −6.687 | −8.357 | 36.143 | 1.00 | 0.84 | 1SG | 2092 |
| ATOM | 2092 | CD1 | ILE | 255 | −7.191 | −9.042 | 34.875 | 1.00 | 0.84 | 1SG | 2093 |
| ATOM | 2093 | C | ILE | 255 | −8.094 | −5.922 | 36.133 | 1.00 | 0.84 | 1SG | 2094 |
| ATOM | 2094 | O | ILE | 255 | −7.984 | −4.914 | 36.832 | 1.00 | 0.84 | 1SG | 2095 |
| ATOM | 2095 | N | ARG | 256 | −7.583 | −5.984 | 34.899 | 1.00 | 0.88 | 1SG | 2096 |
| ATOM | 2096 | CA | ARG | 256 | −6.806 | −4.892 | 34.410 | 1.00 | 0.88 | 1SG | 2097 |
| ATOM | 2097 | CB | ARG | 256 | −6.290 | −5.149 | 32.991 | 1.00 | 0.88 | 1SG | 2098 |
| ATOM | 2098 | CG | ARG | 256 | −5.578 | −6.499 | 32.890 | 1.00 | 0.88 | 1SG | 12099 |
| ATOM | 2099 | CD | ARG | 256 | −4.679 | −6.639 | 31.665 | 1.00 | 0.88 | 1SG | 2100 |
| ATOM | 2100 | NE | ARG | 256 | −3.325 | −6.205 | 32.100 | 1.00 | 0.88 | 1SG | 2101 |
| ATOM | 2101 | CZ | ARG | 256 | −2.481 | −7.100 | 32.687 | 1.00 | 0.88 | 1SG | 2102 |
| ATOM | 2102 | NH1 | ARG | 256 | −2.846 | −8.410 | 32.794 | 1.00 | 0.88 | 1SG | 2103 |
| ATOM | 2103 | NH2 | ARG | 256 | −1.275 | −6.677 | 33.168 | 1.00 | 0.88 | 1SG | 2104 |
| ATOM | 2104 | C | ARG | 256 | −7.681 | −3.686 | 34.395 | 1.00 | 0.88 | 1SG | 2105 |
| ATOM | 2105 | O | ARG | 256 | −7.233 | −2.577 | 34.678 | 1.00 | 0.88 | 1SG | 2106 |
| ATOM | 2106 | N | LEU | 257 | −8.971 | −3.887 | 34.083 | 1.00 | 0.74 | 1SG | 2107 |
| ATOM | 2107 | CA | LEU | 257 | −9.912 | −2.817 | 33.949 | 1.00 | 0.74 | 1SG | 2108 |
| ATOM | 2108 | CB | LEU | 257 | −11.272 | −3.322 | 33.412 | 1.00 | 0.74 | 1SG | 2109 |
| ATOM | 2109 | CG | LEU | 257 | −12.354 | −2.247 | 33.176 | 1.00 | 0.74 | 1SG | 2110 |
| ATOM | 2110 | CD2 | LEU | 257 | −11.831 | −1.119 | 32.269 | 1.00 | 0.74 | 1SG | 2111 |
| ATOM | 2111 | CD1 | LEU | 257 | −12.919 | −1.723 | 34.497 | 1.00 | 0.74 | 1SG | 2112 |
| ATOM | 2112 | C | LEU | 257 | −10.111 | −2.134 | 35.269 | 1.00 | 0.74 | 1SG | 2113 |
| ATOM | 2113 | O | LEU | 257 | −10.251 | −0.913 | 35.308 | 1.00 | 0.74 | 1SG | 2114 |
| ATOM | 2114 | N | GLU | 258 | −10.117 | −2.892 | 36.386 | 1.00 | 0.51 | 1SG | 2115 |
| ATOM | 2115 | CA | GLU | 258 | −10.423 | −2.316 | 37.668 | 1.00 | 0.51 | 1SG | 2116 |
| ATOM | 2116 | CB | GLU | 258 | −10.395 | −3.321 | 38.843 | 1.00 | 0.51 | 1SG | 2117 |
| ATOM | 2117 | CG | GLU | 258 | −8.995 | −3.866 | 39.151 | 1.00 | 0.51 | 1SG | 2118 |
| ATOM | 2118 | CD | GLU | 258 | −9.049 | −4.759 | 40.390 | 1.00 | 0.51 | 1SG | 2119 |
| ATOM | 2119 | OE1 | GLU | 258 | −9.619 | −5.877 | 40.280 | 1.00 | 0.51 | 1SG | 2120 |
| ATOM | 2120 | OE2 | GLU | 258 | −8.515 | −4.341 | 41.455 | 1.00 | 0.51 | 1SG | 2121 |
| ATOM | 2121 | C | GLU | 258 | −9.424 | −1.255 | 38.017 | 1.00 | 0.51 | 1SG | 2122 |
| ATOM | 2122 | O | GLU | 258 | −9.792 | −0.206 | 38.542 | 1.00 | 0.51 | 1SG | 2123 |
| ATOM | 2123 | N | GLU | 259 | −8.132 | −1.476 | 37.721 | 1.00 | 0.33 | 1SG | 2124 |
| ATOM | 2124 | CA | GLU | 259 | −7.145 | −0.524 | 38.152 | 1.00 | 0.33 | 1SG | 2125 |
| ATOM | 2125 | CB | GLU | 259 | −5.704 | −0.905 | 37.767 | 1.00 | 0.33 | 1SG | 2126 |
| ATOM | 2126 | CG | GLU | 259 | −5.458 | −1.008 | 36.259 | 1.00 | 0.33 | 1SG | 2127 |
| ATOM | 2127 | CD | GLU | 259 | −4.003 | −1.410 | 36.039 | 1.00 | 0.33 | 1SG | 2128 |
| ATOM | 2128 | OE1 | GLU | 259 | −3.097 | −0.610 | 36.396 | 1.00 | 0.33 | 1SG | 2129 |
| ATOM | 2129 | OE2 | GLU | 259 | −3.781 | −2.533 | 35.511 | 1.00 | 0.33 | 1SG | 2130 |
| ATOM | 2130 | C | GLU | 259 | −7.428 | 0.818 | 37.554 | 1.00 | 0.33 | 1SG | 2131 |
| ATOM | 2131 | O | GLU | 259 | −7.266 | 1.840 | 38.220 | 1.00 | 0.33 | 1SG | 2132 |
| ATOM | 2132 | N | LEU | 260 | −7.872 | 0.855 | 36.283 | 1.00 | 0.50 | 1SG | 2133 |
| ATOM | 2133 | CA | LEU | 260 | −8.084 | 2.101 | 35.605 | 1.00 | 0.50 | 1SG | 2134 |
| ATOM | 2134 | CB | LEU | 260 | −8.501 | 1.931 | 34.138 | 1.00 | 0.50 | 1SG | 2135 |
| ATOM | 2135 | CG | LEU | 260 | −7.363 | 1.394 | 33.253 | 1.00 | 0.50 | 1SG | 2136 |
| ATOM | 2136 | CD2 | LEU | 260 | −6.865 | 0.028 | 33.738 | 1.00 | 0.50 | 1SG | 2137 |
| ATOM | 2137 | CD1 | LEU | 260 | −6.220 | 2.413 | 33.129 | 1.00 | 0.50 | 1SG | 2138 |
| ATOM | 2138 | C | LEU | 260 | −9.138 | 2.921 | 36.271 | 1.00 | 0.50 | 1SG | 2139 |
| ATOM | 2139 | O | LEU | 260 | −8.970 | 4.131 | 36.415 | 1.00 | 0.50 | 1SG | 2140 |
| ATOM | 2140 | N | HIS | 261 | −10.252 | 2.311 | 36.715 | 1.00 | 0.79 | 1SG | 2141 |
| ATOM | 2141 | CA | HIS | 261 | −11.271 | 3.157 | 37.259 | 1.00 | 0.79 | 1SG | 2142 |
| ATOM | 2142 | ND1 | HIS | 261 | −12.880 | 1.368 | 39.754 | 1.00 | 0.79 | 1SG | 2143 |
| ATOM | 2143 | CG | HIS | 261 | −12.669 | 1.291 | 38.396 | 1.00 | 0.79 | 1SG | 2144 |
| ATOM | 2144 | CB | HIS | 261 | −12.646 | 2.476 | 37.474 | 1.00 | 0.79 | 1SG | 2145 |
| ATOM | 2145 | NE2 | HIS | 261 | −12.653 | −0.789 | 39.268 | 1.00 | 0.79 | 1SG | 2146 |
| ATOM | 2146 | CD2 | HIS | 261 | −12.534 | −0.033 | 38.115 | 1.00 | 0.79 | 1SG | 2147 |
| ATOM | 2147 | CE1 | HIS | 261 | −12.861 | 0.094 | 40.226 | 1.00 | 0.79 | 1SG | 2148 |
| ATOM | 2148 | C | HIS | 261 | −10.787 | 3.784 | 38.525 | 1.00 | 0.79 | 1SG | 2149 |
| ATOM | 2149 | O | HIS | 261 | −9.926 | 3.256 | 39.226 | 1.00 | 0.79 | 1SG | 2150 |
| ATOM | 2150 | N | SER | 262 | −11.334 | 4.979 | 38.814 | 1.00 | 0.86 | 1SG | 2151 |
| ATOM | 2151 | CA | SER | 262 | −11.006 | 5.765 | 39.964 | 1.00 | 0.86 | 1SG | 2152 |
| ATOM | 2152 | CB | SER | 262 | −11.701 | 7.141 | 39.868 | 1.00 | 0.86 | 1SG | 2153 |
| ATOM | 2153 | OG | SER | 262 | −11.356 | 8.004 | 40.941 | 1.00 | 0.86 | 1SG | 2154 |
| ATOM | 2154 | C | SER | 262 | −11.530 | 5.018 | 41.153 | 1.00 | 0.86 | 1SG | 2155 |
| ATOM | 2155 | O | SER | 262 | −11.647 | 3.792 | 41.122 | 1.00 | 0.86 | 1SG | 2156 |
| ATOM | 2156 | N | GLN | 263 | −11.823 | 5.736 | 42.252 | 1.00 | 0.82 | 1SG | 2157 |
| ATOM | 2157 | CA | GLN | 263 | −12.416 | 5.131 | 43.406 | 1.00 | 0.82 | 1SG | 2158 |
| ATOM | 2158 | CB | GLN | 263 | −13.602 | 4.227 | 42.986 | 1.00 | 0.82 | 1SG | 2159 |
| ATOM | 2159 | CG | GLN | 263 | −14.814 | 4.208 | 43.925 | 1.00 | 0.82 | 1SG | 2160 |
| ATOM | 2160 | CD | GLN | 263 | −15.875 | 3.354 | 43.250 | 1.00 | 0.82 | 1SG | 2161 |
| ATOM | 2161 | OE1 | GLN | 263 | −16.643 | 3.849 | 42.424 | 1.00 | 0.82 | 1SG | 2162 |
| ATOM | 2162 | NE2 | GLN | 263 | −15.907 | 2.038 | 43.591 | 1.00 | 0.82 | 1SG | 2163 |
| ATOM | 2163 | C | GLN | 263 | −11.281 | 4.427 | 44.116 | 1.00 | 0.82 | 1SG | 2164 |
| ATOM | 2164 | O | GLN | 263 | −10.167 | 4.935 | 44.049 | 1.00 | 0.82 | 1SG | 2165 |
| ATOM | 2165 | N | PRO | 264 | −11.434 | 3.317 | 44.797 | 1.00 | 0.74 | 1SG | 2166 |
| ATOM | 2166 | CA | PRO | 264 | −10.252 | 2.823 | 45.451 | 1.00 | 0.74 | 1SG | 2167 |
| ATOM | 2167 | CD | PRO | 264 | −12.606 | 3.084 | 45.622 | 1.00 | 0.74 | 1SG | 2168 |
| ATOM | 2168 | CB | PRO | 264 | −10.705 | 2.137 | 46.744 | 1.00 | 0.74 | 1SG | 2169 |
| ATOM | 2169 | CG | PRO | 264 | −12.215 | 1.939 | 46.567 | 1.00 | 0.74 | 1SG | 2170 |
| ATOM | 2170 | C | PRO | 264 | −9.414 | 1.925 | 44.610 | 1.00 | 0.74 | 1SG | 2171 |
| ATOM | 2171 | O | PRO | 264 | −9.932 | 1.293 | 43.692 | 1.00 | 0.74 | 1SG | 2172 |
| ATOM | 2172 | N | THR | 265 | −8.104 | 1.892 | 44.913 | 1.00 | 0.86 | 1SG | 2173 |
| ATOM | 2173 | CA | THR | 265 | −7.141 | 0.978 | 44.377 | 1.00 | 0.86 | 1SG | 2174 |
| ATOM | 2174 | CB | THR | 265 | −7.626 | −0.445 | 44.384 | 1.00 | 0.86 | 1SG | 2175 |
| ATOM | 2175 | OG1 | THR | 265 | −8.031 | −0.797 | 45.699 | 1.00 | 0.86 | 1SG | 2176 |
| ATOM | 2176 | CG2 | THR | 265 | −6.470 | −1.374 | 43.967 | 1.00 | 0.86 | 1SG | 2177 |
| ATOM | 2177 | C | THR | 265 | −6.666 | 1.355 | 43.010 | 1.00 | 0.86 | 1SG | 2178 |
| ATOM | 2178 | O | THR | 265 | −6.779 | 0.566 | 42.072 | 1.00 | 0.86 | 1SG | 2179 |
| ATOM | 2179 | N | PHE | 266 | −6.112 | 2.575 | 42.846 | 1.00 | 1.05 | 1SG | 2180 |
| ATOM | 2180 | CA | PHE | 266 | −5.504 | 2.835 | 41.574 | 1.00 | 1.05 | 1SG | 2181 |
| ATOM | 2181 | CB | PHE | 266 | −4.717 | 4.155 | 41.513 | 1.00 | 1.05 | 1SG | 2182 |
| ATOM | 2182 | CG | PHE | 266 | −5.552 | 5.351 | 41.239 | 1.00 | 1.05 | 1SG | 2183 |
| ATOM | 2183 | CD1 | PHE | 266 | −5.844 | 5.690 | 39.937 | 1.00 | 1.05 | 1SG | 2184 |
| ATOM | 2184 | CD2 | PHE | 266 | −6.006 | 6.146 | 42.265 | 1.00 | 1.05 | 1SG | 2185 |
| ATOM | 2185 | CE1 | PHE | 266 | −6.596 | 6.804 | 39.660 | 1.00 | 1.05 | 1SG | 2186 |
| ATOM | 2186 | CE2 | PHE | 266 | −6.760 | 7.263 | 41.991 | 1.00 | 1.05 | 1SG | 2187 |
| ATOM | 2187 | CZ | PHE | 266 | −7.057 | 7.590 | 40.689 | 1.00 | 1.05 | 1SG | 2188 |
| ATOM | 2188 | C | PHE | 266 | −4.391 | 1.852 | 41.453 | 1.00 | 1.05 | 1SG | 2189 |
| ATOM | 2189 | O | PHE | 266 | −4.376 | 0.971 | 40.594 | 1.00 | 1.05 | 1SG | 2190 |
| ATOM | 2190 | N | PHE | 267 | −3.434 | 2.005 | 42.390 | 1.00 | 0.98 | 1SG | 2191 |
| ATOM | 2191 | CA | PHE | 267 | −2.226 | 1.253 | 42.533 | 1.00 | 0.98 | 1SG | 2192 |
| ATOM | 2192 | CB | PHE | 267 | −1.234 | 1.406 | 41.358 | 1.00 | 0.98 | 1SG | 2193 |
| ATOM | 2193 | CG | PHE | 267 | 0.114 | 0.975 | 41.850 | 1.00 | 0.98 | 1SG | 2194 |
| ATOM | 2194 | CD1 | PHE | 267 | 0.373 | −0.351 | 42.120 | 1.00 | 0.98 | 1SG | 2195 |
| ATOM | 2195 | CD2 | PHE | 267 | 1.112 | 1.905 | 42.086 | 1.00 | 0.98 | 1SG | 2196 |
| ATOM | 2196 | CE1 | PHE | 267 | 1.609 | −0.743 | 42.583 | 1.00 | 0.98 | 1SG | 2197 |
| ATOM | 2197 | CE2 | PHE | 267 | 2.352 | 1.516 | 42.548 | 1.00 | 0.98 | 1SG | 2198 |
| ATOM | 2198 | CZ | PHE | 267 | 2.601 | 0.186 | 42.799 | 1.00 | 0.98 | 1SG | 2199 |
| ATOM | 2199 | C | PHE | 267 | −1.589 | 1.770 | 43.775 | 1.00 | 0.98 | 1SG | 2200 |
| ATOM | 2200 | O | PHE | 267 | −0.928 | 2.811 | 43.798 | 1.00 | 0.98 | 1SG | 2201 |
| ATOM | 2201 | N | PRO | 268 | −1.819 | 1.037 | 44.819 | 1.00 | 0.85 | 1SG | 2202 |
| ATOM | 2202 | CA | PRO | 268 | −1.316 | 1.460 | 46.094 | 1.00 | 0.85 | 1SG | 2203 |
| ATOM | 2203 | CD | PRO | 268 | −3.116 | 0.388 | 44.944 | 1.00 | 0.85 | 1SG | 2204 |
| ATOM | 2204 | CB | PRO | 268 | −2.142 | 0.712 | 47.138 | 1.00 | 0.85 | 1SG | 2205 |
| ATOM | 2205 | CG | PRO | 268 | −3.487 | 0.484 | 46.430 | 1.00 | 0.85 | 1SG | 2206 |
| ATOM | 2206 | C | PRO | 268 | 0.149 | 1.238 | 46.221 | 1.00 | 0.85 | 1SG | 2207 |
| ATOM | 2207 | O | PRO | 268 | 0.719 | 0.473 | 45.447 | 1.00 | 0.85 | 1SG | 2208 |
| ATOM | 2208 | N | GLY | 269 | 0.781 | 1.913 | 47.191 | 1.00 | 0.64 | 1SG | 2209 |
| ATOM | 2209 | CA | GLY | 269 | 2.188 | 1.746 | 47.370 | 1.00 | 0.64 | 1SG | 2210 |
| ATOM | 2210 | C | GLY | 269 | 2.863 | 2.869 | 46.666 | 1.00 | 0.64 | 1SG | 2211 |
| ATOM | 2211 | O | GLY | 269 | 4.059 | 3.092 | 46.845 | 1.00 | 0.64 | 1SG | 2212 |
| ATOM | 2212 | N | GLY | 270 | 2.087 | 3.615 | 45.858 | 1.00 | 0.64 | 1SG | 2213 |
| ATOM | 2213 | CA | GLY | 270 | 2.629 | 4.688 | 45.084 | 1.00 | 0.64 | 1SG | 2214 |
| ATOM | 2214 | C | GLY | 270 | 3.053 | 5.807 | 45.978 | 1.00 | 0.64 | 1SG | 2215 |
| ATOM | 2215 | O | GLY | 270 | 4.166 | 6.319 | 45.857 | 1.00 | 0.64 | 1SG | 2216 |
| ATOM | 2216 | N | LEU | 271 | 2.181 | 6.197 | 46.928 | 1.00 | 1.15 | 1SG | 2217 |
| ATOM | 2217 | CA | LEU | 271 | 2.446 | 7.370 | 47.708 | 1.00 | 1.15 | 1SG | 2218 |
| ATOM | 2218 | CB | LEU | 271 | 1.184 | 8.092 | 48.216 | 1.00 | 1.15 | 1SG | 2219 |
| ATOM | 2219 | CG | LEU | 271 | 0.331 | 8.690 | 47.078 | 1.00 | 1.15 | 1SG | 2220 |
| ATOM | 2220 | CD2 | LEU | 271 | 1.214 | 9.452 | 46.078 | 1.00 | 1.15 | 1SG | 2221 |
| ATOM | 2221 | CD1 | LEU | 271 | −0.839 | 9.532 | 47.617 | 1.00 | 1.15 | 1SG | 2222 |
| ATOM | 2222 | C | LEU | 271 | 3.323 | 7.093 | 48.871 | 1.00 | 1.15 | 1SG | 2223 |
| ATOM | 2223 | O | LEU | 271 | 4.272 | 6.306 | 48.793 | 1.00 | 1.15 | 1SG | 2224 |
| ATOM | 2224 | N | ARG | 272 | 3.017 | 7.793 | 49.983 | 1.00 | 1.54 | 1SG | 2225 |
| ATOM | 2225 | CA | ARG | 272 | 3.858 | 7.711 | 51.128 | 1.00 | 1.54 | 1SG | 2226 |
| ATOM | 2226 | CB | ARG | 272 | 3.418 | 8.550 | 52.336 | 1.00 | 1.54 | 1SG | 2227 |
| ATOM | 2227 | CG | ARG | 272 | 4.229 | 8.201 | 53.586 | 1.00 | 1.54 | 1SG | 2228 |
| ATOM | 2228 | CD | ARG | 272 | 4.356 | 9.336 | 54.605 | 1.00 | 1.54 | 1SG | 2229 |
| ATOM | 2229 | NE | ARG | 272 | 5.585 | 10.103 | 54.262 | 1.00 | 1.54 | 1SG | 2230 |
| ATOM | 2230 | CZ | ARG | 272 | 5.555 | 11.043 | 53.278 | 1.00 | 1.54 | 1SG | 2231 |
| ATOM | 2231 | NH1 | ARG | 272 | 4.401 | 11.269 | 52.584 | 1.00 | 1.54 | 1SG | 2232 |
| ATOM | 2232 | NH2 | ARG | 272 | 6.697 | 11.728 | 52.976 | 1.00 | 1.54 | 1SG | 2233 |
| ATOM | 2233 | C | ARG | 272 | 4.046 | 6.301 | 51.543 | 1.00 | 1.54 | 1SG | 2234 |
| ATOM | 2234 | O | ARG | 272 | 3.169 | 5.646 | 52.109 | 1.00 | 1.54 | 1SG | 2235 |
| ATOM | 2235 | N | PHE | 273 | 5.246 | 5.805 | 51.204 | 1.00 | 1.50 | 1SG | 2236 |
| ATOM | 2236 | CA | PHE | 273 | 5.697 | 4.516 | 51.599 | 1.00 | 1.50 | 1SG | 2237 |
| ATOM | 2237 | CB | PHE | 273 | 5.821 | 4.365 | 53.124 | 1.00 | 1.50 | 1SG | 2238 |
| ATOM | 2238 | CG | PHE | 273 | 6.977 | 5.236 | 53.494 | 1.00 | 1.50 | 1SG | 2239 |
| ATOM | 2239 | CD1 | PHE | 273 | 6.814 | 6.599 | 53.577 | 1.00 | 1.50 | 1SG | 2240 |
| ATOM | 2240 | CD2 | PHE | 273 | 8.219 | 4.703 | 53.757 | 1.00 | 1.50 | 1SG | 2241 |
| ATOM | 2241 | CE1 | PHE | 273 | 7.863 | 7.424 | 53.909 | 1.00 | 1.50 | 1SG | 2242 |
| ATOM | 2242 | CE2 | PHE | 273 | 9.273 | 5.524 | 54.088 | 1.00 | 1.50 | 1SG | 2243 |
| ATOM | 2243 | CZ | PHE | 273 | 9.097 | 6.884 | 54.167 | 1.00 | 1.50 | 1SG | 2244 |
| ATOM | 2244 | C | PHE | 273 | 4.850 | 3.443 | 51.020 | 1.00 | 1.50 | 1SG | 2245 |
| ATOM | 2245 | O | PHE | 273 | 3.874 | 3.683 | 50.312 | 1.00 | 1.50 | 1SG | 2246 |
| ATOM | 2246 | N | SER | 274 | 5.259 | 2.215 | 51.368 | 1.00 | 1.41 | 1SG | 2247 |
| ATOM | 2247 | CA | SER | 274 | 4.779 | 0.955 | 50.911 | 1.00 | 1.41 | 1SG | 2248 |
| ATOM | 2248 | CB | SER | 274 | 3.783 | 1.009 | 49.739 | 1.00 | 1.41 | 1SG | 2249 |
| ATOM | 2249 | OG | SER | 274 | 2.521 | 1.454 | 50.215 | 1.00 | 1.41 | 1SG | 2250 |
| ATOM | 2250 | C | SER | 274 | 6.049 | 0.340 | 50.476 | 1.00 | 1.41 | 1SG | 2251 |
| ATOM | 2251 | O | SER | 274 | 6.710 | 0.860 | 49.576 | 1.00 | 1.41 | 1SG | 2252 |
| ATOM | 2252 | N | VAL | 275 | 6.427 | −0.757 | 51.148 | 1.00 | 1.25 | 1SG | 2253 |
| ATOM | 2253 | CA | VAL | 275 | 7.723 | −1.346 | 51.016 | 1.00 | 1.25 | 1SG | 2254 |
| ATOM | 2254 | CB | VAL | 275 | 7.819 | −2.695 | 51.645 | 1.00 | 1.25 | 1SG | 2255 |
| ATOM | 2255 | CG1 | VAL | 275 | 7.616 | −2.525 | 53.162 | 1.00 | 1.25 | 1SG | 2256 |
| ATOM | 2256 | CG2 | VAL | 275 | 6.779 | −3.605 | 50.967 | 1.00 | 1.25 | 1SG | 2257 |
| ATOM | 2257 | C | VAL | 275 | 8.139 | −1.447 | 49.597 | 1.00 | 1.25 | 1SG | 2258 |
| ATOM | 2258 | O | VAL | 275 | 7.382 | −1.831 | 48.711 | 1.00 | 1.25 | 1SG | 2259 |
| ATOM | 2259 | N | CYS | 276 | 9.378 | −0.986 | 49.380 | 1.00 | 0.99 | 1SG | 2260 |
| ATOM | 2260 | CA | CYS | 276 | 10.098 | −1.018 | 48.149 | 1.00 | 0.99 | 1SG | 2261 |
| ATOM | 2261 | CB | CYS | 276 | 9.708 | 0.094 | 47.161 | 1.00 | 0.99 | 1SG | 2262 |
| ATOM | 2262 | SG | CYS | 276 | 10.280 | 1.733 | 47.705 | 1.00 | 0.99 | 1SG | 2263 |
| ATOM | 2263 | C | CYS | 276 | 11.469 | −0.697 | 48.628 | 1.00 | 0.99 | 1SG | 2264 |
| ATOM | 2264 | O | CYS | 276 | 11.695 | −0.651 | 49.836 | 1.00 | 0.99 | 1SG | 2265 |
| ATOM | 2265 | N | LEU | 277 | 12.444 | −0.477 | 47.730 | 1.00 | 0.98 | 1SG | 2266 |
| ATOM | 2266 | CA | LEU | 277 | 13.693 | −0.071 | 48.292 | 1.00 | 0.98 | 1SG | 2267 |
| ATOM | 2267 | CB | LEU | 277 | 14.902 | −0.326 | 47.373 | 1.00 | 0.98 | 1SG | 2268 |
| ATOM | 2268 | CG | LEU | 277 | 15.155 | −1.835 | 47.173 | 1.00 | 0.98 | 1SG | 2269 |
| ATOM | 2269 | CD2 | LEU | 277 | 16.505 | −2.098 | 46.487 | 1.00 | 0.98 | 1SG | 2270 |
| ATOM | 2270 | CD1 | LEU | 277 | 13.972 | −2.510 | 46.457 | 1.00 | 0.98 | 1SG | 2271 |
| ATOM | 2271 | C | LEU | 277 | 13.533 | 1.383 | 48.575 | 1.00 | 0.98 | 1SG | 2272 |
| ATOM | 2272 | O | LEU | 277 | 13.916 | 2.245 | 47.783 | 1.00 | 0.98 | 1SG | 2273 |
| ATOM | 2273 | N | PHE | 278 | 12.938 | 1.671 | 49.749 | 1.00 | 1.06 | 1SG | 2274 |
| ATOM | 2274 | CA | PHE | 278 | 12.594 | 2.997 | 50.166 | 1.00 | 1.06 | 1SG | 2275 |
| ATOM | 2275 | CB | PHE | 278 | 11.957 | 3.039 | 51.566 | 1.00 | 1.06 | 1SG | 2276 |
| ATOM | 2276 | CG | PHE | 278 | 12.901 | 2.427 | 52.541 | 1.00 | 1.06 | 1SG | 2277 |
| ATOM | 2277 | CD1 | PHE | 278 | 13.013 | 1.056 | 52.608 | 1.00 | 1.06 | 1SG | 2278 |
| ATOM | 2278 | CD2 | PHE | 278 | 13.652 | 3.212 | 53.389 | 1.00 | 1.06 | 1SG | 2279 |
| ATOM | 2279 | CE1 | PHE | 278 | 13.875 | 0.465 | 53.505 | 1.00 | 1.06 | 1SG | 2280 |
| ATOM | 2280 | CE2 | PHE | 278 | 14.514 | 2.630 | 54.290 | 1.00 | 1.06 | 1SG | 2281 |
| ATOM | 2281 | CZ | PHE | 278 | 14.622 | 1.261 | 54.343 | 1.00 | 1.06 | 1SG | 2282 |
| ATOM | 2282 | C | PHE | 278 | 13.783 | 3.896 | 50.147 | 1.00 | 1.06 | 1SG | 2283 |
| ATOM | 2283 | O | PHE | 278 | 13.745 | 4.963 | 49.535 | 1.00 | 1.06 | 1SG | 2284 |
| ATOM | 2284 | N | ARG | 279 | 14.885 | 3.495 | 50.799 | 1.00 | 1.02 | 1SG | 2285 |
| ATOM | 2285 | CA | ARG | 279 | 16.004 | 4.392 | 50.870 | 1.00 | 1.02 | 1SG | 2286 |
| ATOM | 2286 | CB | ARG | 279 | 17.143 | 3.893 | 51.773 | 1.00 | 1.02 | 1SG | 2287 |
| ATOM | 2287 | CG | ARG | 279 | 18.513 | 4.453 | 51.379 | 1.00 | 1.02 | 1SG | 2288 |
| ATOM | 2288 | CD | ARG | 279 | 18.617 | 5.980 | 51.396 | 1.00 | 1.02 | 1SG | 2289 |
| ATOM | 2289 | NE | ARG | 279 | 18.946 | 6.413 | 52.781 | 1.00 | 1.02 | 1SG | 2290 |
| ATOM | 2290 | CZ | ARG | 279 | 17.947 | 6.821 | 53.618 | 1.00 | 1.02 | 1SG | 2291 |
| ATOM | 2291 | NH1 | ARG | 279 | 16.651 | 6.788 | 53.194 | 1.00 | 1.02 | 1SG | 2292 |
| ATOM | 2292 | NH2 | ARG | 279 | 18.257 | 7.258 | 54.871 | 1.00 | 1.02 | 1SG | 2293 |
| ATOM | 2293 | C | ARG | 279 | 16.594 | 4.644 | 49.520 | 1.00 | 1.02 | 1SG | 2294 |
| ATOM | 2294 | O | ARG | 279 | 17.304 | 3.803 | 48.972 | 1.00 | 1.02 | 1SG | 295 |
| ATOM | 2295 | N | ARG | 280 | 16.305 | 5.830 | 48.943 | 1.00 | 0.98 | 1SG | 2296 |
| ATOM | 2296 | CA | ARG | 280 | 16.933 | 6.174 | 47.700 | 1.00 | 0.98 | 1SG | 2297 |
| ATOM | 2297 | CB | ARG | 280 | 16.263 | 5.509 | 46.486 | 1.00 | 0.98 | 1SG | 2298 |
| ATOM | 2298 | CG | ARG | 280 | 14.789 | 5.856 | 46.279 | 1.00 | 0.98 | 1SG | 2299 |
| ATOM | 2299 | CD | ARG | 280 | 14.546 | 7.078 | 45.393 | 1.00 | 0.98 | 1SG | 2300 |
| ATOM | 2300 | NE | ARG | 280 | 14.232 | 6.560 | 44.030 | 1.00 | 0.98 | 1SG | 2301 |
| ATOM | 2301 | CZ | ARG | 280 | 13.774 | 7.411 | 43.064 | 1.00 | 0.98 | 1SG | 2302 |
| ATOM | 2302 | NH1 | ARG | 280 | 13.709 | 8.750 | 43.319 | 1.00 | 0.98 | 1SG | 2303 |
| ATOM | 2303 | NH2 | ARG | 280 | 13.371 | 6.925 | 41.854 | 1.00 | 0.98 | 1SG | 2304 |
| ATOM | 2304 | C | ARG | 280 | 16.928 | 7.663 | 47.567 | 1.00 | 0.98 | 1SG | 2305 |
| ATOM | 2305 | O | ARG | 280 | 15.878 | 8.303 | 47.606 | 1.00 | 0.98 | 1SG | 2306 |
| ATOM | 2306 | N | ILE | 281 | 18.132 | 8.247 | 47.404 | 1.00 | 0.90 | 1SG | 2307 |
| ATOM | 2307 | CA | ILE | 281 | 18.277 | 9.673 | 47.345 | 1.00 | 0.90 | 1SG | 2308 |
| ATOM | 2308 | CB | ILE | 281 | 19.597 | 10.130 | 47.910 | 1.00 | 0.90 | 1sg | 2309 |
| ATOM | 2309 | CG2 | ILE | 281 | 19.718 | 11.657 | 47.774 | 1.00 | 0.90 | 1SG | 2310 |
| ATOM | 2310 | CG1 | ILE | 281 | 19.724 | 9.662 | 49.367 | 1.00 | 0.90 | 1sg | 2311 |
| ATOM | 2311 | CG1 | ILE | 281 | 18.652 | 10.248 | 50.284 | 1.00 | 0.90 | 1sG | 2312 |
| ATOM | 2312 | C | ILE | 281 | 18.196 | 10.097 | 45.914 | 1.00 | 0.90 | 1SG | 2313 |
| ATOM | 2313 | O | ILE | 281 | 18.444 | 9.308 | 45.003 | 1.00 | 0.90 | 1SG | 2314 |
| ATOM | 2314 | N | VAL | 282 | 17.830 | 11.378 | 45.702 | 1.00 | 0.72 | 1SG | 2315 |
| ATOM | 2315 | CA | VAL | 282 | 17.690 | 11.973 | 44.405 | 1.00 | 0.72 | 1SG | 2316 |
| ATOM | 2316 | CB | VAL | 282 | 17.345 | 13.435 | 44.480 | 1.00 | 0.72 | 1sg | 2317 |
| ATOM | 2317 | CG1 | VAL | 282 | 18.551 | 14.186 | 45.073 | 1.00 | 0.72 | 1SG | 2318 |
| ATOM | 2318 | CG2 | VAL | 282 | 16.913 | 13.941 | 43.093 | 1.00 | 0.72 | 1sg | 2319 |
| ATOM | 2319 | C | VAL | 282 | 18.993 | 11.851 | 43.689 | 1.00 | 0.72 | 1SG | 2320 |
| ATOM | 2320 | O | VAL | 282 | 20.000 | 11.441 | 44.265 | 1.00 | 0.72 | 1SG | 2321 |
| ATOM | 2321 | N | ALA | 283 | 18.983 | 12.167 | 42.381 | 1.00 | 0.60 | 1SG | 2322 |
| ATOM | 2322 | CA | ALA | 283 | 20.162 | 12.105 | 41.578 | 1.00 | 0.60 | 1SG | 2323 |
| ATOM | 2323 | CB | ALA | 283 | 19.877 | 12.356 | 40.086 | 1.00 | 0.60 | 1SG | 2324 |
| ATOM | 2324 | C | ALA | 283 | 21.111 | 13.156 | 42.039 | 1.00 | 0.60 | 1SG | 2325 |
| ATOM | 2325 | O | ALA | 283 | 22.304 | 12.898 | 42.183 | 1.00 | 0.60 | 1SG | 2326 |
| ATOM | 2326 | N | CYS | 284 | 20.594 | 14.369 | 42.316 | 1.00 | 0.72 | 1SG | 2327 |
| ATOM | 2327 | CA | CYS | 284 | 21.468 | 15.450 | 42.658 | 1.00 | 0.72 | 1SG | 2328 |
| ATOM | 2328 | CB | CYS | 284 | 20.762 | 16.814 | 42.760 | 1.00 | 0.72 | 1SG | 2329 |
| ATOM | 2329 | SG | CYS | 284 | 21.911 | 18.154 | 43.196 | 1.00 | 0.72 | 1SG | 2330 |
| ATOM | 2330 | C | CYS | 284 | 22.107 | 15.163 | 43.973 | 1.00 | 0.72 | 1SG | 2331 |
| ATOM | 2331 | O | CYS | 284 | 21.424 | 14.890 | 44.959 | 1.00 | 0.72 | 1SG | 2332 |
| ATOM | 2332 | N | HIS | 285 | 23.455 | 15.207 | 43.970 | 1.00 | 0.86 | 1SG | 2333 |
| ATOM | 2333 | CA | HIS | 285 | 24.333 | 15.004 | 45.086 | 1.00 | 0.86 | 1SG | 2334 |
| ATOM | 2334 | ND1 | HIS | 285 | 24.557 | 15.655 | 48.336 | 1.00 | 0.86 | 1SG | 2335 |
| ATOM | 2335 | CG | HIS | 285 | 24.570 | 14.518 | 47.559 | 1.00 | 0.86 | 1SG | 2336 |
| ATOM | 2336 | CB | HIS | 285 | 23.716 | 14.344 | 46.333 | 1.00 | 0.86 | 1SG | 2337 |
| ATOM | 2337 | NE2 | HIS | 285 | 26.027 | 14.272 | 49.268 | 1.00 | 0.86 | 1SG | 2338 |
| ATOM | 2338 | CD2 | HIS | 285 | 25.474 | 13.683 | 48.144 | 1.00 | 0.86 | 1SG | 2339 |
| ATOM | 2339 | CE1 | HIS | 285 | 25.445 | 15.455 | 49.342 | 1.00 | 0.86 | 1SG | 2340 |
| ATOM | 2340 | C | HIS | 285 | 25.388 | 14.086 | 44.575 | 1.00 | 0.86 | 1SG | 2341 |
| ATOM | 2341 | O | HIS | 285 | 25.914 | 14.278 | 43.480 | 1.00 | 0.86 | 1SG | 2342 |
| ATOM | 2342 | N | PHE | 286 | 25.724 | 13.056 | 45.368 | 1.00 | 0.81 | 1SG | 2343 |
| ATOM | 2343 | CA | PHE | 286 | 26.691 | 12.103 | 44.929 | 1.00 | 0.81 | 1SG | 2344 |
| ATOM | 2344 | CB | PHE | 286 | 27.105 | 11.129 | 46.046 | 1.00 | 0.81 | 1SG | 2345 |
| ATOM | 2345 | CG | PHE | 286 | 28.267 | 10.320 | 45.583 | 1.00 | 0.81 | 1SG | 2346 |
| ATOM | 2346 | CD1 | PHE | 286 | 29.533 | 10.861 | 45.578 | 1.00 | 0.81 | 1SG | 2347 |
| ATOM | 2347 | CD2 | PHE | 286 | 28.099 | 9.016 | 45.176 | 1.00 | 0.81 | 1SG | 2348 |
| ATOM | 2348 | CE1 | PHE | 286 | 30.612 | 10.120 | 45.159 | 1.00 | 0.81 | 1SG | 2349 |
| ATOM | 2349 | CE2 | PHE | 286 | 29.175 | 8.271 | 44.756 | 1.00 | 0.81 | 1SG | 2350 |
| ATOM | 2350 | CZ | PHE | 286 | 30.434 | 8.822 | 44.744 | 1.00 | 0.81 | 1SG | 2351 |
| ATOM | 2351 | C | PHE | 286 | 25.999 | 11.343 | 43.849 | 1.00 | 0.81 | 1SG | 2352 |
| ATOM | 2352 | O | PHE | 286 | 24.816 | 11.561 | 43.595 | 1.00 | 0.81 | 1SG | 2353 |
| ATOM | 2353 | N | ILE | 287 | 26.714 | 10.435 | 43.165 | 1.00 | 0.75 | 1SG | 2354 |
| ATOM | 2354 | CA | ILE | 287 | 26.064 | 9.724 | 42.107 | 1.00 | 0.75 | 1SG | 2355 |
| ATOM | 2355 | CB | ILE | 287 | 27.011 | 8.907 | 41.267 | 1.00 | 0.75 | 1SG | 2356 |
| ATOM | 2356 | CG2 | ILE | 287 | 27.714 | 7.876 | 42.165 | 1.00 | 0.75 | 1SG | 2357 |
| ATOM | 2357 | CG1 | ILE | 287 | 26.282 | 8.312 | 40.051 | 1.00 | 0.75 | 1SG | 2358 |
| ATOM | 2358 | CD1 | ILE | 287 | 27.227 | 7.753 | 38.987 | 1.00 | 0.75 | 1SG | 2359 |
| ATOM | 2359 | C | ILE | 287 | 25.020 | 8.842 | 42.715 | 1.00 | 0.75 | 1SG | 2360 |
| ATOM | 2360 | O | ILE | 287 | 25.319 | 7.941 | 43.497 | 1.00 | 0.75 | 1SG | 2361 |
| ATOM | 2361 | N | LYS | 288 | 23.743 | 9.120 | 42.380 | 1.00 | 0.80 | 1SG | 2362 |
| ATOM | 2362 | CA | LYS | 288 | 22.637 | 8.372 | 42.899 | 1.00 | 0.80 | 1SG | 2363 |
| ATOM | 2363 | CB | LYS | 288 | 21.800 | 9.219 | 43.871 | 1.00 | 0.80 | 1SG | 2364 |
| ATOM | 2364 | CG | LYS | 288 | 22.548 | 9.490 | 45.179 | 1.00 | 0.80 | 1SG | 2365 |
| ATOM | 2365 | CD | LYS | 288 | 21.922 | 10.587 | 46.042 | 1.00 | 0.80 | 1SG | 2366 |
| ATOM | 2366 | CE | LYS | 288 | 22.660 | 10.838 | 47.361 | 1.00 | 0.80 | 1SG | 2367 |
| ATOM | 2367 | NZ | LYS | 288 | 22.447 | 9.711 | 48.298 | 1.00 | 0.80 | 1SG | 2368 |
| ATOM | 2368 | C | LYS | 288 | 21.810 | 7.963 | 41.719 | 1.00 | 0.80 | 1SG | 2369 |
| ATOM | 2369 | O | LYS | 288 | 22.335 | 7.892 | 40.610 | 1.00 | 0.80 | 1SG | 2370 |
| ATOM | 2370 | N | PRO | 289 | 20.547 | 7.663 | 41.883 | 1.00 | 0.96 | 1SG | 2371 |
| ATOM | 2371 | CA | PRO | 289 | 19.787 | 7.321 | 40.711 | 1.00 | 0.96 | 1SG | 2372 |
| ATOM | 2372 | CD | PRO | 289 | 20.113 | 6.868 | 43.021 | 1.00 | 0.96 | 1SG | 2373 |
| ATOM | 2373 | CB | PRO | 289 | 18.591 | 6.494 | 41.186 | 1.00 | 0.96 | 1SG | 2374 |
| ATOM | 2374 | CG | PRO | 289 | 18.638 | 6.572 | 42.721 | 1.00 | 0.96 | 1SG | 2375 |
| ATOM | 2375 | C | PRO | 289 | 19.409 | 8.560 | 39.977 | 1.00 | 0.96 | 1SG | 2376 |
| ATOM | 2376 | O | PRO | 289 | 19.594 | 9.645 | 40.521 | 1.00 | 0.96 | 1SG | 2377 |
| ATOM | 2377 | N | ARG | 290 | 18.885 | 8.423 | 38.746 | 1.00 | 0.95 | 1SG | 2378 |
| ATOM | 2378 | CA | ARG | 290 | 18.505 | 9.556 | 37.954 | 1.00 | 0.95 | 1SG | 2379 |
| ATOM | 2379 | CB | ARG | 290 | 17.769 | 10.654 | 38.739 | 1.00 | 0.95 | 1SG | 2380 |
| ATOM | 2380 | CG | ARG | 290 | 17.385 | 11.835 | 37.844 | 1.00 | 0.95 | 1SG | 2381 |
| ATOM | 2381 | CD | ARG | 290 | 16.685 | 12.986 | 38.565 | 1.00 | 0.95 | 1SG | 2382 |
| ATOM | 2382 | NE | ARG | 290 | 16.464 | 14.057 | 37.554 | 1.00 | 0.95 | 1SG | 2383 |
| ATOM | 2383 | CZ | ARG | 290 | 15.911 | 15.248 | 37.928 | 1.00 | 0.95 | 1SG | 2384 |
| ATOM | 2384 | NH1 | ARG | 290 | 15.565 | 15.464 | 39.230 | 1.00 | 0.95 | 1SG | 2385 |
| ATOM | 2385 | NH2 | ARG | 290 | 15.710 | 16.223 | 36.995 | 1.00 | 0.95 | 1SG | 2386 |
| ATOM | 2386 | C | ARG | 290 | 19.733 | 10.169 | 37.372 | 1.00 | 0.95 | 1SG | 2387 |
| ATOM | 2387 | O | ARG | 290 | 19.669 | 10.840 | 36.344 | 1.00 | 0.95 | 1SG | 2388 |
| ATOM | 2388 | N | THR | 291 | 20.901 | 9.933 | 37.994 | 1.00 | 0.84 | 1SG | 2389 |
| ATOM | 2389 | CA | THR | 291 | 22.090 | 10.505 | 37.440 | 1.00 | 0.84 | 1SG | 2390 |
| ATOM | 2390 | CB | THR | 291 | 23.303 | 10.370 | 38.313 | 1.00 | 0.84 | 1SG | 2391 |
| ATOM | 2391 | OG1 | THR | 291 | 23.610 | 9.001 | 38.524 | 1.00 | 0.84 | 1SG | 2392 |
| ATOM | 2392 | CG2 | THR | 291 | 23.015 | 11.059 | 39.655 | 1.00 | 0.84 | 1SG | 2393 |
| ATOM | 2393 | C | THR | 291 | 22.363 | 9.804 | 36.153 | 1.00 | 0.84 | 1SG | 2394 |
| ATOM | 2394 | O | THR | 291 | 21.549 | 9.010 | 35.683 | 1.00 | 0.84 | 1SG | 2395 |
| ATOM | 2395 | N | LEU | 292 | 23.525 | 10.099 | 35.537 | 1.00 | 1.02 | 1SG | 2396 |
| ATOM | 2396 | CA | LEU | 292 | 23.853 | 9.475 | 34.292 | 1.00 | 1.02 | 1SG | 2397 |
| ATOM | 2397 | CB | LEU | 292 | 25.253 | 9.842 | 33.778 | 1.00 | 1.02 | 1SG | 2398 |
| ATOM | 2398 | CG | LEU | 292 | 25.611 | 9.177 | 32.438 | 1.00 | 1.02 | 1SG | 2399 |
| ATOM | 2399 | CD2 | LEU | 292 | 27.099 | 9.347 | 32.102 | 1.00 | 1.02 | 1SG | 2400 |
| ATOM | 2400 | CD1 | LEU | 292 | 24.684 | 9.664 | 31.311 | 1.00 | 1.02 | 1SG | 2401 |
| ATOM | 2401 | C | LEU | 292 | 23.832 | 8.009 | 34.549 | 1.00 | 1.02 | 1SG | 2402 |
| ATOM | 2402 | O | LEU | 292 | 23.177 | 7.253 | 33.835 | 1.00 | 1.02 | 1SG | 2403 |
| ATOM | 2403 | N | LEU | 293 | 24.540 | 7.579 | 35.602 | 1.00 | 1.13 | 1SG | 2404 |
| ATOM | 2404 | CA | LEU | 293 | 24.512 | 6.194 | 35.941 | 1.00 | 1.13 | 1SG | 2405 |
| ATOM | 2405 | CB | LEU | 293 | 25.880 | 5.629 | 36.355 | 1.00 | 1.13 | 1SG | 2406 |
| ATOM | 2406 | CG | LEU | 293 | 25.838 | 4.137 | 36.732 | 1.00 | 1.13 | 1SG | 2407 |
| ATOM | 2407 | CD2 | LEU | 293 | 27.166 | 3.689 | 37.364 | 1.00 | 1.13 | 1SG | 2408 |
| ATOM | 2408 | CG1 | LEU | 293 | 25.434 | 3.277 | 35.517 | 1.00 | 1.13 | 1SG | 2409 |
| ATOM | 2409 | C | LEU | 293 | 23.610 | 6.087 | 37.123 | 1.00 | 1.13 | 1SG | 2410 |
| ATOM | 2410 | O | LEU | 293 | 23.980 | 6.466 | 38.232 | 1.00 | 1.13 | 1SG | 2411 |
| ATOM | 2411 | N | ASP | 294 | 22.382 | 5.576 | 36.913 | 1.00 | 0.84 | 1SG | 2412 |
| ATOM | 2412 | CA | ASP | 294 | 21.466 | 5.472 | 38.005 | 1.00 | 0.84 | 1SG | 2413 |
| ATOM | 2413 | CB | ASP | 294 | 20.119 | 4.829 | 37.638 | 1.00 | 0.84 | 1SG | 2414 |
| ATOM | 2414 | CG | ASP | 294 | 20.369 | 3.402 | 37.175 | 1.00 | 0.84 | 1SG | 2415 |
| ATOM | 2415 | OD1 | ASP | 294 | 21.460 | 3.141 | 36.603 | 1.00 | 0.84 | 1SG | 2416 |
| ATOM | 2416 | OD2 | ASP | 294 | 19.466 | 2.550 | 37.386 | 1.00 | 0.84 | 1SG | 2417 |
| ATOM | 2417 | C | ASP | 294 | 22.114 | 4.624 | 39.040 | 1.00 | 0.84 | 1SG | 2418 |
| ATOM | 2418 | O | ASP | 294 | 22.796 | 3.650 | 38.725 | 1.00 | 0.84 | 1SG | 2419 |
| ATOM | 2419 | N | TYR | 295 | 21.946 | 5.002 | 40.317 | 1.00 | 0.51 | 1SG | 2420 |
| ATOM | 2420 | CA | TYR | 295 | 22.554 | 4.231 | 41.354 | 1.00 | 0.51 | 1SG | 2421 |
| ATOM | 2421 | CB | TYR | 295 | 23.285 | 5.112 | 42.383 | 1.00 | 0.51 | 1SG | 2422 |
| ATOM | 2422 | CG | TYR | 295 | 24.266 | 4.275 | 43.128 | 1.00 | 0.51 | 1SG | 2423 |
| ATOM | 2423 | CD1 | TYR | 295 | 25.507 | 4.044 | 42.581 | 1.00 | 0.51 | 1SG | 2424 |
| ATOM | 2424 | CD2 | TYR | 295 | 23.964 | 3.736 | 44.356 | 1.00 | 0.51 | 1SG | 2425 |
| ATOM | 2425 | CE1 | TYR | 295 | 26.439 | 3.284 | 43.244 | 1.00 | 0.51 | 1SG | 2426 |
| ATOM | 2426 | CE2 | TYR | 295 | 24.893 | 2.973 | 45.025 | 1.00 | 0.51 | 1SG | 2427 |
| ATOM | 2427 | CZ | TYR | 295 | 26.131 | 2.747 | 44.470 | 1.00 | 0.51 | 1SG | 2428 |
| ATOM | 2428 | OH | TYR | 295 | 27.085 | 1.966 | 45.155 | 1.00 | 0.51 | 1SG | 2429 |
| ATOM | 2429 | C | TYR | 295 | 21.412 | 3.557 | 42.037 | 1.00 | 0.51 | 1SG | 2430 |
| ATOM | 2430 | O | TYR | 295 | 20.274 | 4.006 | 41.916 | 1.00 | 0.51 | 1SG | 2431 |
| ATOM | 2431 | N | TRP | 296 | 21.680 | 2.452 | 42.761 | 1.00 | 0.34 | 1SG | 2432 |
| ATOM | 2432 | CA | TRP | 296 | 20.615 | 1.762 | 43.431 | 1.00 | 0.34 | 1SG | 2433 |
| ATOM | 2433 | CB | TRP | 296 | 19.964 | 2.623 | 44.526 | 1.00 | 0.34 | 1SG | 2434 |
| ATOM | 2434 | CG | TRP | 296 | 20.882 | 2.899 | 45.696 | 1.00 | 0.34 | 1SG | 2435 |
| ATOM | 2435 | CD2 | TRP | 296 | 20.795 | 4.059 | 46.537 | 1.00 | 0.34 | 1SG | 2436 |
| ATOM | 2436 | CD1 | TRP | 296 | 21.936 | 2.165 | 46.157 | 1.00 | 0.34 | 1SG | 2437 |
| ATOM | 2437 | NE1 | TRP | 296 | 22.509 | 2.793 | 47.237 | 1.00 | 0.34 | 1SG | 2438 |
| ATOM | 2438 | CE2 | TRP | 296 | 21.818 | 3.962 | 47.481 | 1.00 | 0.34 | 1SG | 2439 |
| ATOM | 2439 | CE3 | TRP | 296 | 19.937 | 5.122 | 46.522 | 1.00 | 0.34 | 1SG | 2440 |
| ATOM | 2440 | CZ2 | TRP | 296 | 21.999 | 4.929 | 48.427 | 1.00 | 0.34 | 1SG | 2441 |
| ATOM | 2441 | CZ3 | TRP | 296 | 20.119 | 6.094 | 47.481 | 1.00 | 0.34 | 1SG | 2442 |
| ATOM | 2442 | CH2 | TRP | 296 | 21.130 | 5.999 | 48.414 | 1.00 | 0.34 | 1SG | 2443 |
| ATOM | 2443 | C | TRP | 296 | 19.588 | 1.404 | 42.404 | 1.00 | 0.34 | 1SG | 2444 |
| ATOM | 2444 | O | TRP | 296 | 18.432 | 1.817 | 42.472 | 1.00 | 0.34 | 1SG | 2445 |
| ATOM | 2445 | N | GLN | 297 | 20.017 | 0.598 | 41.420 | 1.00 | 0.25 | 1SG | 2446 |
| ATOM | 2446 | CA | GLN | 297 | 19.226 | 0.177 | 40.302 | 1.00 | 0.25 | 1SG | 2447 |
| ATOM | 2447 | CB | GLN | 297 | 20.029 | −0.726 | 39.349 | 1.00 | 0.25 | 1SG | 2448 |
| ATOM | 2448 | CG | GLN | 297 | 21.285 | −0.053 | 38.792 | 1.00 | 0.25 | 1SG | 2449 |
| ATOM | 2449 | CD | GLN | 297 | 22.062 | −1.095 | 38.000 | 1.00 | 0.25 | 1SG | 2450 |
| ATOM | 2450 | OE1 | GLN | 297 | 23.215 | −0.881 | 37.629 | 1.00 | 0.25 | 1SG | 2451 |
| ATOM | 2451 | NE2 | GLN | 297 | 21.415 | −2.263 | 37.739 | 1.00 | 0.25 | 1SG | 2452 |
| ATOM | 2452 | C | GLN | 297 | 18.070 | −0.633 | 40.793 | 1.00 | 0.25 | 1SG | 2453 |
| ATOM | 2453 | O | GLN | 297 | 16.961 | −0.589 | 40.222 | 1.00 | 0.25 | 1SG | 2454 |
| ATOM | 2454 | N | ALA | 298 | 18.2 | −1.375 | 41.892 | 1.00 | 0.36 | 1SG | 2455 |
| ATOM | 2455 | CA | ALA | 298 | 17.30 | −2.324 | 42.343 | 1.00 | 0.36 | 1SG | 2456 |
| ATOM | 2456 | CB | ALA | 298 | 17.715 | −3.014 | 43.653 | 1.00 | 0.36 | 1SG | 2457 |
| ATOM | 2457 | C | ALA | 298 | 15.971 | −1.683 | 42.583 | 1.00 | 0.36 | 1SG | 2458 |
| ATOM | 2458 | O | ALA | 298 | 14.953 | −2.218 | 42.146 | 1.00 | 0.36 | 1SG | 2459 |
| ATOM | 2459 | N | LEU | 299 | 15.912 | −0.519 | 43.257 | 1.00 | 0.52 | 1SG | 2460 |
| ATOM | 2460 | CA | LEU | 299 | 14.597 | −0.015 | 43.542 | 1.00 | 0.52 | 1SG | 2461 |
| ATOM | 2461 | CB | LEU | 299 | 14.524 | 0.960 | 44.745 | 1.00 | 0.52 | 1SG | 2462 |
| ATOM | 2462 | CG | LEU | 299 | 15.556 | 2.110 | 44.795 | 1.00 | 0.52 | 1SG | 2463 |
| ATOM | 2463 | CD2 | LEU | 299 | 15.295 | 3.194 | 43.742 | 1.00 | 0.52 | 1SG | 2464 |
| ATOM | 2464 | CD1 | LEU | 299 | 16.996 | 1.578 | 44.813 | 1.00 | 0.52 | 1SG | 2465 |
| ATOM | 2465 | C | LEU | 299 | 13.989 | 0.587 | 42.316 | 1.00 | 0.52 | 1SG | 2466 |
| ATOM | 2466 | O | LEU | 299 | 14.513 | 1.526 | 41.721 | 1.00 | 0.52 | 1SG | 2467 |
| ATOM | 2467 | N | GLU | 300 | 12.851 | 0.010 | 41.882 | 1.00 | 0.37 | 1SG | 2468 |
| ATOM | 2468 | CA | GLU | 300 | 12.188 | 0.494 | 40.708 | 1.00 | 0.37 | 1SG | 2469 |
| ATOM | 2469 | CB | GLU | 300 | 10.953 | −0.338 | 40.321 | 1.00 | 0.37 | 1SG | 2470 |
| ATOM | 2470 | CG | GLU | 300 | 11.269 | −1.750 | 39.828 | 1.00 | 0.37 | 1SG | 2471 |
| ATOM | 2471 | CD | GLU | 300 | 9.947 | −2.425 | 39.488 | 1.00 | 0.37 | 1SG | 2472 |
| ATOM | 2472 | OE1 | GLU | 300 | 8.886 | −1.767 | 39.662 | 1.00 | 0.37 | 1SG | 2473 |
| ATOM | 2473 | OE2 | GLU | 300 | 9.979 | −3.606 | 39.049 | 1.00 | 0.37 | 1SG | 2474 |
| ATOM | 2474 | C | GLU | 300 | 11.692 | 1.874 | 40.980 | 1.00 | 0.37 | 1SG | 2475 |
| ATOM | 2475 | O | GLU | 300 | 11.994 | 2.813 | 40.244 | 1.00 | 0.37 | 1SG | 2476 |
| ATOM | 2476 | N | ASN | 301 | 10.922 | 2.032 | 42.072 | 1.00 | 0.35 | 1SG | 2477 |
| ATOM | 2477 | CA | ASN | 301 | 10.384 | 3.319 | 42.395 | 1.00 | 0.35 | 1SG | 2478 |
| ATOM | 2478 | CB | ASN | 301 | 8.887 | 3.471 | 42.068 | 1.00 | 0.35 | 1SG | 2479 |
| ATOM | 2479 | CG | ASN | 301 | 8.700 | 3.487 | 40.559 | 1.00 | 0.35 | 1SG | 2480 |
| ATOM | 2480 | OD1 | ASN | 301 | 9.161 | 2.595 | 39.849 | 1.00 | 0.35 | 1SG | 2481 |
| ATOM | 2481 | ND2 | ASN | 301 | 7.994 | 4.534 | 40.055 | 1.00 | 0.35 | 1SG | 2482 |
| ATOM | 2482 | C | ASN | 301 | 10.490 | 3.471 | 43.872 | 1.00 | 0.35 | 1SG | 2483 |
| ATOM | 2483 | O | ASN | 301 | 10.397 | 2.495 | 44.615 | 1.00 | 0.35 | 1SG | 2484 |
| ATOM | 2484 | N | SER | 302 | 10.701 | 4.710 | 44.348 | 1.00 | 0.57 | 1SG | 2485 |
| ATOM | 2485 | CA | SER | 302 | 10.770 | 4.854 | 45.767 | 1.00 | 0.57 | 1SG | 2486 |
| ATOM | 2486 | CB | SER | 302 | 12.111 | 4.395 | 46.361 | 1.00 | 0.57 | 1SG | 2487 |
| ATOM | 2487 | OG | SER | 302 | 11.994 | 4.284 | 47.772 | 1.00 | 0.57 | 1SG | 2488 |
| ATOM | 2488 | C | SER | 302 | 10.568 | 6.299 | 46.086 | 1.00 | 0.57 | 1SG | 2489 |
| ATOM | 2489 | O | SER | 302 | 9.892 | 7.019 | 45.350 | 1.00 | 0.57 | 1SG | 2490 |
| ATOM | 2490 | N | ARG | 303 | 11.154 | 6.741 | 47.216 | 1.00 | 0.69 | 1SG | 2491 |
| ATOM | 2491 | CA | ARG | 303 | 11.073 | 8.084 | 47.714 | 1.00 | 0.69 | 1SG | 2492 |
| ATOM | 2492 | CB | ARG | 303 | 12.134 | 8.349 | 48.805 | 1.00 | 0.69 | 1SG | 2493 |
| ATOM | 2493 | CG | ARG | 303 | 12.336 | 9.806 | 49.232 | 1.00 | 0.69 | 1SG | 2494 |
| ATOM | 2494 | CD | ARG | 303 | 13.624 | 10.400 | 48.658 | 1.00 | 0.69 | 1SG | 2495 |
| ATOM | 2495 | HE | ARG | 303 | 13.988 | 11.590 | 49.476 | 1.00 | 0.69 | 1SG | 2496 |
| ATOM | 2496 | CZ | ARG | 303 | 13.614 | 12.843 | 49.090 | 1.00 | 0.69 | 1SG | 2497 |
| ATOM | 2497 | NH1 | ARG | 303 | 12.861 | 13.017 | 47.965 | 1.00 | 0.69 | 1SG | 2498 |
| ATOM | 2498 | NH2 | ARG | 303 | 14.011 | 13.922 | 49.828 | 1.00 | 0.69 | 1SG | 2499 |
| ATOM | 2499 | C | ARG | 303 | 11.286 | 9.033 | 46.581 | 1.00 | 0.69 | 1SG | 2500 |
| ATOM | 2500 | O | ARG | 303 | 12.252 | 8.911 | 45.830 | 1.00 | 0.69 | 1SG | 2501 |
| ATOM | 2501 | N | GLY | 304 | 10.360 | 10.006 | 46.437 | 1.00 | 0.73 | 1SG | 2502 |
| ATOM | 2502 | CA | GLY | 304 | 10.423 | 10.979 | 45.387 | 1.00 | 0.73 | 1SG | 2503 |
| ATOM | 2503 | C | GLY | 304 | 9.446 | 12.050 | 45.744 | 1.00 | 0.73 | 1SG | 2504 |
| ATOM | 2504 | O | GLY | 304 | 8.351 | 11.768 | 46.228 | 1.00 | 0.73 | 1SG | 2505 |
| ATOM | 2505 | N | GLU | 305 | 9.025 | 13.318 | 45.504 | 1.00 | 0.80 | 1SG | 2506 |
| ATOM | 2506 | CA | GLU | 305 | 8.969 | 14.412 | 45.853 | 1.00 | 0.80 | 1SG | 507 |
| ATOM | 2507 | CB | GLU | 305 | 9.647 | 15.405 | 46.814 | 1.00 | 0.80 | 1SG | 2508 |
| ATOM | 2508 | CG | GLU | 305 | 8.774 | 16.590 | 47.227 | 1.00 | 0.80 | 1SG | 2509 |
| ATOM | 2509 | CD | GLU | 305 | 9.595 | 17.437 | 48.191 | 1.00 | 0.80 | 1SG | 2510 |
| ATOM | 2510 | OE1 | GLU | 305 | 10.787 | 17.091 | 48.409 | 1.00 | 0.80 | 1SG | 2511 |
| ATOM | 2511 | OE2 | GLU | 305 | 9.044 | 18.437 | 48.723 | 1.00 | 0.80 | 1SG | 2512 |
| ATOM | 2512 | C | GLU | 305 | 8.627 | 15.146 | 44.597 | 1.00 | 0.80 | 1sg | 2513 |
| ATOM | 2513 | O | GLU | 305 | 9.230 | 14.929 | 43.547 | 1.00 | 0.80 | 1SG | 2514 |
| ATOM | 2514 | N | ASP | 306 | 7.608 | 16.024 | 44.677 | 1.00 | 0.80 | 1SG | 2515 |
| ATOM | 2515 | CA | ASP | 306 | 7.215 | 16.812 | 43.547 | 1.00 | 0.80 | 1SG | 2516 |
| ATOM | 2516 | CB | ASP | 306 | 5.967 | 17.676 | 43.812 | 1.00 | 0.80 | 1SG | 2517 |
| ATOM | 2517 | CG | ASP | 306 | 5.545 | 18.349 | 42.508 | 1.00 | 0.80 | 1SG | 2518 |
| ATOM | 2518 | OD1 | ASP | 306 | 6.204 | 18.096 | 41.465 | 1.00 | 0.80 | 1SG | 2519 |
| ATOM | 2519 | OD2 | ASP | 306 | 4.555 | 19.129 | 42.541 | 1.00 | 0.80 | 1SG | 2520 |
| ATOM | 2520 | C | ASP | 306 | 8.349 | 17.739 | 43.260 | 1.00 | 0.80 | 1SG | 2521 |
| ATOM | 2521 | O | ASP | 306 | 8.660 | 18.030 | 42.106 | 1.00 | 0.80 | 1SG | 2522 |
| ATOM | 2522 | N | CYS | 307 | 9.019 | 18.208 | 44.327 | 1.00 | 0.74 | 1SG | 2523 |
| ATOM | 2523 | CA | CYS | 307 | 10.098 | 19.137 | 44.175 | 1.00 | 0.74 | 1SG | 2524 |
| ATOM | 2524 | CB | CYS | 307 | 10.800 | 19.463 | 45.504 | 1.00 | 0.74 | 1SG | 2525 |
| ATOM | 2525 | SG | CYS | 307 | 9.709 | 20.349 | 46.655 | 1.00 | 0.74 | 1SG | 2526 |
| ATOM | 2526 | C | CYS | 307 | 11.109 | 18.540 | 43.250 | 1.00 | 0.74 | 1SG | 2527 |
| ATOM | 2527 | O | CYS | 307 | 11.659 | 19.239 | 42.402 | 1.00 | 0.74 | 1SG | 2528 |
| ATOM | 2528 | N | PRO | 308 | 11.379 | 17.272 | 43.364 | 1.00 | 0.83 | 1SG | 2529 |
| ATOM | 2529 | CA | PRO | 308 | 12.337 | 16.707 | 42.462 | 1.00 | 0.83 | 1SG | 2530 |
| ATOM | 2530 | CD | PRO | 308 | 11.426 | 16.617 | 44.659 | 1.00 | 0.83 | 1SG | 2531 |
| ATOM | 2531 | CB | PRO | 308 | 12.848 | 15.419 | 43.114 | 1.00 | 0.83 | 1SG | 2532 |
| ATOM | 2532 | CG | PRO | 308 | 11.932 | 15.204 | 44.333 | 1.00 | 0.83 | 1SG | 2533 |
| ATOM | 2533 | C | PRO | 308 | 11.714 | 16.515 | 41.125 | 1.00 | 0.83 | 1SG | 2534 |
| ATOM | 2534 | O | PRO | 308 | 10.496 | 16.368 | 41.038 | 1.00 | 0.83 | 1SG | 2535 |
| ATOM | 2535 | N | PRO | 309 | 12.517 | 16.546 | 40.106 | 1.00 | 1.05 | 1SG | 2536 |
| ATOM | 2536 | CA | PRO | 309 | 11.969 | 16.361 | 38.796 | 1.00 | 1.05 | 1SG | 2537 |
| ATOM | 2537 | CD | PRO | 309 | 13.615 | 17.501 | 40.100 | 1.00 | 1.05 | 1SG | 2538 |
| ATOM | 2538 | CB | PRO | 309 | 12.977 | 16.967 | 37.824 | 1.00 | 1.05 | 1SG | 2539 |
| ATOM | 2539 | CG | PRO | 309 | 13.692 | 18.039 | 38.662 | 1.00 | 1.05 | 1SG | 2540 |
| ATOM | 2540 | C | PRO | 309 | 11.713 | 14.913 | 38.562 | 1.00 | 1.05 | 1SG | 2541 |
| ATOM | 2541 | O | PRO | 309 | 12.330 | 14.083 | 39.227 | 1.00 | 1.05 | 1SG | 2542 |
| ATOM | 2542 | N | VAL | 310 | 10.800 | 14.592 | 37.627 | 1.00 | 1.14 | 1SG | 2543 |
| ATOM | 2543 | CA | VAL | 310 | 10.499 | 13.224 | 37.339 | 1.00 | 1.14 | 1SG | 2544 |
| ATOM | 2544 | CB | VAL | 310 | 9.454 | 13.067 | 36.274 | 1.00 | 1.14 | 1SG | 2545 |
| ATOM | 2545 | CG1 | VAL | 310 | 9.251 | 11.568 | 35.996 | 1.00 | 1.14 | 1SG | 2546 |
| ATOM | 2546 | CG2 | VAL | 310 | 8.179 | 13.794 | 36.733 | 1.00 | 1.14 | 1SG | 2547 |
| ATOM | 2547 | C | VAL | 310 | 11.778 | 12.571 | 36.830 | 1.00 | 1.14 | 1SG | 2548 |
| ATOM | 2548 | O | VAL | 310 | 12.225 | 11.577 | 37.462 | 1.00 | 1.14 | 1SG | 2549 |
| ATOM | 2549 | OXT | VAL | 310 | 12.322 | 13.053 | 35.800 | 1.00 | 1.14 | 1SG | 2550 |
| END | | | | | | | | | | | |

TABLE 9
Consensus polar and hydrophobic interactions in UDP binding sites of glycosyltransferases-UDP complexes:

| Protein | Distance between protein atom and the corresponding ligand atom in A | | | | | | | | |
|---|---|---|---|---|---|---|---|---|---|
| Di- | Uracil | | | | Ribose | | | | |
| phosphate | O2 | N3 | O4 | Ring | O2(H) | O3(H) | Ring | PO11 | PO21 |
| GnTI* | His190 ND1 | Asp144 OD1 | Lys186NZ | Cys11-Cys145 | Asp212OD1 | Asp212OD1 | Ile113 | Asp213OD1 | Asp213OD1 |
| | 2.7 | 2.8 | 5.0 | Ile187 3.8 | 2.8 | 3.1 | 4.2 | 4.0 | 4.2 |
| SpSA** | | Asp39OD1 | Arg71NH1 | Tyr11 | Asp98OD2 | Asp98OD2 | Thr9 | Asp99OD1 | Asp99OD1 |
| | | 2.7 | 3.0 | 3.5 | 3.0 | 3.3 | 4.5 | 3.6 | 4.5 |
| Core2 | His131 ND1 | Asp99 OD1 | | Cys5-Cys100 | Asp160OD1 | Asp160OD1 | Ile57 | Glu159OE1 | Glu159OE2 |
| | 3.1 | 2.9 | | Val128, Ile133 | 2.9 | 3.1 | 4.1 | Glu252OE1 | Glu253NH |
| | | | | 3.3, 3.9, 4.0 | | | | 3.7, 3.6 | 3.7, 2.5 |

TABLE 10

| Atomic | Atomic Contact on | Atomic Contact on | Distance Between | Nature of |
|---|---|---|---|---|
| Interaction | Nucleotide Sugar Donor | Enzyme | Atomic Contacts | Interaction |
| 1 | Uracil O2 | His-190 ND1 | 2.7 ± 0.5 | H8 |
| 2 | Uracil N3 | Asp-144 OD1 | 2.8 ± 0.5 | HB |
| 3 | Uracil Ring (stacking) | Cys-115-Cys-145 | 4.2 ± 0.5 | HP |
| | | Ile-187 | 3.8 ± 0.5 | HP |
| 4 | Uracil O4 | Lys-186 NZ | 4.5 ± 0.5 | HB |
| 5 | Ribose ring (stacking) | Ile-113, Ala-114 | 4.2 ± 0.5 | HP |
| 6 | Ribose O3′ (H) and | Asp-212 OD1 | 2.8 ± 0.5 | HB |
| | O2′ (H) | | 3.1 ± 0.5 | HB |
| 7 | Phosphoryl(O5′-P)O | GIu-211 OE1 | 4.2 ± 0.5 | MM |
| 8 | GIcNAcNac | Leu-331 | 3.2 ± 0.5 | HP |
| 9 | Nac | Leu-269 | 3.3 ± 0.5 | HP |
| 10 | O4 | Trp-290 NE1 | 2.7 ± 0.5 | HB |
| 11 | O3 | Glu 211 | 2.7 ± 0.5 | HB |
| 12 | O4 | Glu 211 | 2.7 ± 0.5 | HB |

TABLE 11

| Atomic | Atomic Contact on Nucleotide | |
|---|---|---|
| Interaction | Sugar Donor | Atomic Contact on Enzyme |
| 2 | Uracil O2 (HB) | Asn 406 ND2 |
| | Uracil O2 (weak HB) | His 368 NE2 |
| 3 | Uracil N3 (HB) | Gln 370 OE1 |
| 4 | Uracil Ring (stacking) | Val 331 |
| 5 | Ribose O2 (HB) | His 430 NE2 |
| 6 | Uridine (HI) | Asp 333 |
| 7 | | Pro 407 |
| 8 | Pyrophosphate | Glu-428 via Mn²⁺ |
| 9 | GlcNAc O6 (HB) | Ser 543 N |
| 10 | O6 (HB) | Lys 542 N |
| 11 | O4 (HB) | Ser 544 N |
| 12 | HI contact | Pro 541 |
| 13 | | Trp 403 |

TABLE 12

| | | | Distance | | |
|---|---|---|---|---|---|
| | Atomic Contacts on | | Between Core2L | | Atomic Contact on |
| Atomic | Nucleotide | Atomic Contact on | and | Nature of | Model |
| Interaction | Sugar Donor | Core2L | UDP-GlcNAc | interaction | (Table 3, 4, and 5) |
| 1 | Uracil O2 | His-131 ND1 | 3.1 ± 0.5 | HB | His-84 ND1 |
| 2 | Uracil N3 | Asp-99 OD1 | 2.9 ± 0.5 | HB | Asp-52 OD1 |
| 3 | Uracil Ring (stacking) | Cys-59-Cys-100- | 3.3 ± 0.5 | HI | Cys-12-Cys-53- |
| 4 | | Val-128 | 3.9 ± 0.5 | HI | Val-81 |
| 5 | | Ile-133 | 4.0 ± 0.5 | HI | Ile-86 |
| 6 | Ribose ring (stacking) | Ile-57 | 4.1 ± 0.5 | HI | Ile-10 |
| 7 | Ribose O3′(H) and | Asp-160 OD1 | 2.9 ± 0.5 | HB | Asp-113 OD1 |
| | O2′(H) | | 3.1 ± 0.5 | HB | |
| 8 | Ribose O3′ | Glu-159 OE1 | 3.1 ± 0.5 | HB | Glu-112 OE1 |
| 9 | Phosphoryl (O5′-P)O | Glu-159-OE2 (Mn) | 3.4 ± 0.5 | MMI | Glu-112-OE2 (Mn) |
| 10 | POa | Glu-159 OE2 (Mn) | 3.6 ± 0.5 | MMI | Glu-112 OE2 (Mn) |
| 11 | PO | Glu-159 OE2 (Mn) | 2.9 ± 0.5 | MMI | Glu-112 OE2 (Mn) |
| 12 | POa | Glu-253 NH (loop) | 2.9 ± 0.5 | HB | Glu-206 NH (loop) |
| 13 | POb | Glu 252 OE1(loop) (Mn) | 2.7 ± 0.5 | MMI | Glu 205 OE1(loop) (Mn) |
| 14 | GlcNAc Nac | Leu-163 | 4.0 ± 0.5 | HI | Leu-116 |
| 15 | Nac | Val-128 | 2.5 ± 0.5 | HI | Val-81 |
| 16 | O4(H) | Ser-207 OG | 3.5 ± 0.5 | HB | Ser-160 OG |
| 17 | O3(H) | Lys-157 NZ | 3.1 ± 0.5 | HB | Lys-110 NZ |
| 18 | Ring | Met-135 SD | 4.5 ± 0.5 | HI | Met-88 SD |

HB: hydrogen bond interaction
MMI: metal mediated interaction
HI: hydrophobic interaction

TABLE 13

| | Atomic Contacts on Nucleotide | Atomic Contacts |
|---|---|---|
| Atomic Interaction | Sugar Donor | on Enzyme |
| 1 | Uridine | Cys-35-Cys-70 |
| 2 | | Asp-100 |
| 3 | | Leu-106 |
| 4 | | His-112 |
| 5 | | Asn-69 |
| 6 | | Thr-101 |
| 7 | | Leu-86 |
| 8 | | Asp-36 |
| 9 | | Glu-127 |
| 10 | | Leu-33 |
| 11 | Pyrophosphate | Glu-128 |
| 12 | | Pro-258 |
| 13 | | Glu-253 |
| 14 | | Met-252 |
| 15 | | Ser-190 |
| | Pyrophosphate | Loop Coordinates |
| 16 | GlcNAc | His-163 |
| 17 | | Leu-192 |

TABLE 14
CLUSTERING HISTOGRAM

| | Lowest | | Mean | Num | |
|---|---|---|---|---|---|
| Cluster | Docked | | Docked | in | Histogram |
| Rank | Energy | Run | Energy | Clus | 5 10 15 20 25 30 35 |
| 1 | −12.68 | 5 | −12.49 | 4 | #### |
| 2 | −11.99 | 10 | −11.84 | 4 | #### |
| 3 | −10.51 | 2 | −10.51 | 1 | # |
| 4 | −10.09 | 9 | −10.09 | 1 | # |

LOWEST ENERGY DOCKED CONFORMATION from EACH CLUSTER
Residue number will be set to the conformation's cluster rank.

| | |
|---|---|
| MODEL | 5 |

| USER | Run = 5 | |
|---|---|---|
| USER | Cluster Rank = 1 | |
| USER | Number of conformations in this cluster = 4 | |
| USER | | |
| USER | RMSD from reference structure | = 2.596 A |
| USER | | |
| USER | Estimated Free Energy of Binding | = −9.82 kcal/mol [= (1) + (3)] |
| USER | Estimated Inhibition Constant, Ki | = +6.34e−08 [Temperature = 298.15 K] |
| USER | | |
| USER | Final Docked Energy | = −12.68 kcal/mol [= (1) + (2)] |
| USER | | |
| USER | (1) Final Intermolecular Energy | = −12.00 kcal/mol |
| USER | (2) Final Internal Energy of Ligand | = −0.68 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran0 −0.755828 18.422711 8.990951 | | | | | | | | | | |
| USER | NEWDPF quat0 −0.930372 −0.207073 −0.302538 −49.521378 | | | | | | | | | | |
| USER | NEWDPF ndihe 7 | | | | | | | | | | |
| USER | NEWDPF dihe0 −68.02 −166.44 180.00 130.66 −11.25 20.96 −12.26 | | | | | | | | | | |
| USER | | | | | | | | | | | |
| USER | | | | Rank | x | y | z | vdW | Elec | q | RMS |
| ATOM | 1 | C1_{—} | UDP | 1 | −0.675 | 18.074 | 7.615 | −0.62 | +0.31 | +0.324 | 2.596 |
| ATOM | 2 | C5 | UDP | 1 | 0.154 | 21.501 | 6.381 | −0.59 | +0.00 | +0.000 | 2.596 |
| ATOM | 3 | C6 | UDP | 1 | 0.198 | 20.325 | 7.027 | −0.51 | +0.00 | +0.000 | 2.596 |
| ATOM | 4 | C2_{—} | UDP | 1 | 0.682 | 17.374 | 7.524 | −0.61 | +0.09 | +0.113 | 2.596 |
| ATOM | 5 | C3_{—} | UDP | 1 | 0.636 | 16.510 | 8.806 | −0.68 | +0.08 | +0.113 | 2.596 |
| ATOM | 6 | C4 | UDP | 1 | −0.904 | 21.757 | 5.455 | −0.67 | +0.25 | +0.396 | 2.596 |
| ATOM | 7 | O4_{—} | UDP | 1 | −0.756 | 18.423 | 8.991 | −0.08 | −0.23 | −0.227 | 2.596 |
| ATOM | 8 | O2 | UDP | 1 | −2.660 | 18.656 | 5.808 | −0.52 | −0.68 | −0.396 | 2.596 |
| ATOM | 9 | O4 | UDP | 1 | −1.084 | 22.780 | 4.795 | −0.13 | −0.26 | −0.396 | 2.596 |
| ATOM | 10 | C4_{—} | UDP | 1 | −0.166 | 17.360 | 9.790 | −0.64 | +0.10 | +0.113 | 2.596 |
| ATOM | 11 | N1 | UDP | 1 | −0.763 | 19.334 | 6.864 | −0.37 | −0.20 | −0.211 | 2.596 |
| ATOM | 12 | C2 | UDP | 1 | −1.796 | 19.518 | 5.987 | −0.78 | +0.39 | +0.396 | 2.596 |
| ATOM | 13 | N3 | UDP | 1 | −1.831 | 20.721 | 5.329 | −0.46 | −0.23 | −0.440 | 2.596 |
| ATOM | 14 | H3 | UDP | 1 | −2.615 | 20.873 | 4.678 | −0.26 | −0.12 | +0.440 | 2.596 |
| ATOM | 15 | O2_{—} | UDP | 1 | 0.735 | 16.544 | 6.388 | −0.22 | −0.31 | −0.537 | 2.596 |
| ATOM | 16 | HO2_{—} | UDP | 1 | 0.506 | 15.576 | 6.658 | −0.09 | +0.04 | +0.424 | 2.596 |
| ATOM | 17 | O3_{—} | UDP | 1 | −0.096 | 15.325 | 8.485 | −0.34 | −0.29 | −0.537 | 2.596 |
| ATOM | 18 | HO3_{—} | UDP | 1 | 0.533 | 14.642 | 8.040 | −0.28 | −0.11 | +0.424 | 2.596 |
| ATOM | 19 | C5_{—} | UDP | 1 | 0.700 | 17.993 | 10.878 | −0.43 | +0.12 | +0.113 | 2.596 |
| ATOM | 20 | O5_{—} | UDP | 1 | 1.861 | 18.516 | 10.261 | +0.00 | −0.42 | −0.368 | 2.596 |
| ATOM | 21 | PA | UDP | 1 | 3.402 | 18.219 | 10.424 | −0.53 | +1.57 | +1.019 | 2.596 |
| ATOM | 22 | O1A | UDP | 1 | 3.588 | 16.741 | 10.365 | −0.64 | −0.51 | −0.255 | 2.596 |
| ATOM | 23 | O2A | UDP | 1 | 4.157 | 19.066 | 9.449 | −0.44 | −0.51 | −0.255 | 2.596 |
| ATOM | 24 | O3A | UDP | 1 | 3.496 | 18.715 | 11.936 | +0.04 | −0.69 | −0.510 | 2.596 |
| ATOM | 25 | PB | UDP | 1 | 3.787 | 20.140 | 12.603 | −0.50 | +1.20 | +1.019 | 2.596 |
| ATOM | 26 | O2B | UDP | 1 | 5.247 | 20.270 | 12.363 | −0.30 | −0.36 | −0.255 | 2.596 |
| ATOM | 27 | O3B | UDP | 1 | 3.234 | 20.304 | 13.972 | +0.05 | −0.23 | −0.255 | 2.596 |
| ATOM | 28 | O1B | UDP | 1 | 2.944 | 21.191 | 11.710 | −0.08 | −0.32 | −0.255 | 2.596 |
| TER | | | | | | | | | | | |

ENDMDL

| | |
|---|---|
| MODEL | 6 |

| USER | Run = 6 | |
|---|---|---|
| USER | Cluster Rank = 1 | |
| USER | Number of conformations in this cluster = 4 | |
| USER | | |
| USER | RMSD from reference structure | = 2.637 A |
| USER | | |
| USER | Estimated Free Energy of Binding | = −9.66 kcal/mol [= (1) + (3)] |
| USER | Estimated Inhibition Constant, Ki | = +8.31e−08 [Temperature = 298.15 K] |
| USER | | |
| USER | Final Docked Energy | = −12.67 kcal/mol [= (1) + (2)] |
| USER | | |
| USER | (1) Final Intermolecular Energy | = −11.84 kcal/mol |
| USER | (2) Final Internal Energy of Ligand | = −0.84 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran −0.451299 18.203106 9.086235 | | | | | | | | | | |
| USER | NEWDPF quat0 0.941373 0.334495 −0.043941 54.667631 | | | | | | | | | | |
| USER | NEWDPF ndihe 7 | | | | | | | | | | |
| USER | NEWDPF dihe −10.49 −173.33 −27.76 −55.41 −66.29 10.40 23.25 | | | | | | | | | | |
| USER | | | | | | | | | | | |
| USER | | | | Rank | x | y | z | vdW | Elec | q | RMS |
| ATOM | 1 | C1_{—} | UDP | 1 | −0.784 | 17.952 | 7.726 | −0.63 | +0.31 | +0.324 | 2.637 |
| ATOM | 2 | C5 | UDP | 1 | 0.457 | 21.216 | 6.400 | −0.52 | +0.00 | +0.000 | 2.637 |
| ATOM | 3 | C6 | UDP | 1 | 0.396 | 20.011 | 6.988 | −0.52 | +0.00 | +0.000 | 2.637 |
| ATOM | 4 | C2_{—} | UDP | 1 | 0.323 | 16.994 | 7.281 | −0.69 | +0.08 | +0.113 | 2.637 |
| ATOM | 5 | C3_{—} | UDP | 1 | 0.399 | 16.063 | 8.513 | −0.69 | +0.07 | +0.113 | 2.637 |
| ATOM | 6 | C4 | UDP | 1 | −0.710 | 21.757 | 5.778 | −0.67 | +0.27 | +0.396 | 2.637 |
| ATOM | 7 | O4_{—} | UDP | 1 | −0.451 | 18.203 | 9.086 | −0.07 | −0.22 | −0.227 | 2.637 |
| ATOM | 8 | O2 | UDP | 1 | −2.967 | 19.070 | 6.492 | −0.47 | −0.78 | −0.396 | 2.637 |
| ATOM | 9 | O4 | UDP | 1 | −0.813 | 22.842 | 5.208 | −0.12 | −0.29 | −0.396 | 2.637 |
| ATOM | 10 | C4_{—} | UDP | 1 | 0.065 | 16.983 | 9.686 | −0.64 | +0.10 | +0.113 | 2.637 |
| ATOM | 11 | N1 | UDP | 1 | −0.771 | 19.256 | 7.050 | −0.37 | −0.20 | −0.211 | 2.637 |
| ATOM | 12 | C2 | UDP | 1 | −1.918 | 19.718 | 6.467 | −0.80 | +0.40 | +0.396 | 2.637 |
| ATOM | 13 | N3 | UDP | 1 | −1.844 | 20.948 | 5.866 | −0.51 | −0.27 | −0.440 | 2.637 |
| ATOM | 14 | H3 | UDP | 1 | −2.708 | 21.310 | 5.437 | −0.25 | +0.02 | +0.440 | 2.637 |
| ATOM | 15 | O2_{—} | UDP | 1 | −0.084 | 16.261 | 6.150 | −0.26 | −0.34 | −0.537 | 2.637 |
| ATOM | 16 | HO2 | UDP | 1 | −1.077 | 16.005 | 6.244 | +0.03 | +0.32 | +0.424 | 2.637 |
| ATOM | 17 | O3_{—} | UDP | 1 | −0.632 | 15.084 | 8.361 | −0.30 | −0.32 | −0.537 | 2.637 |
| ATOM | 18 | HO3_{—} | UDP | 1 | −0.339 | 14.382 | 7.667 | −0.31 | +0.02 | +0.424 | 2.637 |
| ATOM | 19 | C5_{—} | UDP | 1 | 1.286 | 17.337 | 10.535 | −0.46 | +0.11 | +0.113 | 2.637 |
| ATOM | 20 | O5_{—} | UDP | 1 | 1.996 | 18.363 | 9.869 | +0.00 | −0.42 | −0.368 | 2.637 |
| ATOM | 21 | PA | UDP | 1 | 3.511 | 18.549 | 9.470 | −0.55 | +1.63 | +1.019 | 2.637 |
| ATOM | 22 | O1A | UDP | 1 | 4.280 | 17.420 | 10.067 | −0.64 | −0.57 | −0.255 | 2.637 |
| ATOM | 23 | O2A | UDP | 1 | 3.592 | 18.756 | 7.990 | −0.17 | −0.36 | −0.255 | 2.637 |
| ATOM | 24 | O3A | UDP | 1 | 3.737 | 19.871 | 10.331 | −0.26 | −0.88 | −0.510 | 2.637 |
| ATOM | 25 | PB | UDP | 1 | 4.114 | 20.126 | 11.865 | −0.49 | +1.47 | +1.019 | 2.637 |
| ATOM | 26 | O2B | UDP | 1 | 5.576 | 19.861 | 11.843 | −0.33 | −0.39 | −0.255 | 2.637 |
| ATOM | 27 | O3B | UDP | 1 | 3.240 | 19.425 | 12.839 | +0.06 | −0.29 | −0.255 | 2.637 |
| ATOM | 28 | O1B | UDP | 1 | 3.800 | 21.692 | 12.110 | −0.32 | −0.35 | −0.255 | 2.637 |
| TER | | | | | | | | | | | |

ENDMDL

| | |
|---|---|
| MODEL | 3 |

| USER | Run = 3 | |
|---|---|---|
| USER | Cluster Rank = 1 | |
| USER | Number of conformations in this cluster = 4 | |
| USER | | |
| USER | RMSD from reference structure | = 2.692 A |
| USER | | |
| USER | Estimated Free Energy of Binding | = −9.78 kcal/mol [= (1) + (3)] |
| USER | Estimated Inhibition Constant, Ki | = +6.78e−08 [Temperature = 298.15 K] |
| USER | | |
| USER | Final Docked Energy | = −12.49 kcal/mol [= (1) + (2)] |
| USER | | |
| USER | (1) Final Intermolecular Energy | = −11.96 kcal/mol |
| USER | (2) Final Internal Energy of Ligand | = −0.53 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran −0.441262 18.326362 8.972435 | | | | | | | | | | |
| USER | NEWDPF quat0 0.924008 0.380234 −0.040389 54.117552 | | | | | | | | | | |
| USER | NEWDPF ndihe 7 | | | | | | | | | | |
| USER | NEWDPF dihe 172.93 −120.63 −164.74 −29.15 −23.91 −27.01 −0.87 | | | | | | | | | | |
| USER | | | | | | | | | | | |
| USER | | | | Rank | x | y | z | vdW | Elec | q | RMS |
| ATOM | 1 | C1_{—} | UDP | 1 | −0.811 | 18.038 | 7.630 | −0.63 | +0.31 | +0.324 | 2.692 |
| ATOM | 2 | C5 | UDP | 1 | 0.307 | 21.300 | 6.195 | −0.56 | +0.00 | +0.000 | 2.692 |
| ATOM | 3 | C6 | UDP | 1 | 0.294 | 20.106 | 6.809 | −0.54 | +0.00 | +0.000 | 2.692 |
| ATOM | 4 | C2_{—} | UDP | 1 | 0.303 | 17.096 | 7.169 | −0.69 | +0.08 | +0.113 | 2.692 |
| ATOM | 5 | C3_{—} | UDP | 1 | 0.441 | 16.194 | 8.417 | −0.68 | +0.07 | +0.113 | 2.692 |
| ATOM | 6 | C4 | UDP | 1 | −0.892 | 21.799 | 5.599 | −0.68 | +0.26 | +0.396 | 2.692 |
| ATOM | 7 | O4_{—} | UDP | 1 | −0.441 | 18.326 | 8.972 | −0.06 | −0.22 | −0.227 | 2.692 |
| ATOM | 8 | O2 | UDP | 1 | −3.060 | 19.077 | 6.445 | −0.44 | −0.81 | −0.396 | 2.692 |
| ATOM | 9 | O4 | UDP | 1 | −1.040 | 22.869 | 5.010 | −0.13 | −0.27 | −0.396 | 2.692 |
| ATOM | 10 | C4_{—} | UDP | 1 | 0.123 | 17.132 | 9.581 | −0.63 | +0.10 | +0.113 | 2.692 |
| ATOM | 11 | N1 | UDP | 1 | −0.852 | 19.327 | 6.926 | −0.37 | −0.20 | −0.211 | 2.692 |
| ATOM | 12 | C2 | UDP | 1 | −2.028 | 19.748 | 6.371 | −0.81 | +0.40 | +0.396 | 2.692 |
| ATOM | 13 | N3 | UDP | 1 | −2.003 | 20.966 | 5.742 | −0.50 | −0.24 | −0.440 | 2.692 |
| ATOM | 14 | H3 | UDP | 1 | −2.889 | 21.298 | 5.334 | −0.27 | −0.08 | +0.440 | 2.692 |
| ATOM | 15 | O2_{—} | UDP | 1 | −0.123 | 16.329 | 6.068 | −0.24 | −0.36 | −0.537 | 2.692 |
| ATOM | 16 | HO2_{—} | UDP | 1 | 0.669 | 16.181 | 5.424 | +0.02 | +0.27 | +0.424 | 2.692 |
| ATOM | 17 | O3_{—} | UDP | 1 | −0.570 | 15.188 | 8.321 | −0.30 | −0.31 | −0.537 | 2.692 |
| ATOM | 18 | HO3_{—} | UDP | 1 | −0.145 | 14.260 | 8.455 | −0.36 | +0.29 | +0.424 | 2.692 |
| ATOM | 19 | C5_{—} | UDP | 1 | 1.362 | 17.534 | 10.381 | −0.51 | +0.12 | +0.113 | 2.692 |
| ATOM | 20 | O5_{—} | UDP | 1 | 2.294 | 18.104 | 9.483 | −0.02 | −0.44 | −0.368 | 2.692 |
| ATOM | 21 | PA | UDP | 1 | 3.824 | 18.474 | 9.600 | −0.55 | +1.76 | +1.019 | 2.692 |
| ATOM | 22 | O1A | UDP | 1 | 4.480 | 17.433 | 10.442 | −0.66 | −0.61 | −0.255 | 2.692 |
| ATOM | 23 | O2A | UDP | 1 | 4.362 | 18.714 | 8.225 | −0.31 | −0.47 | −0.255 | 2.692 |
| ATOM | 24 | O3A | UDP | 1 | 3.597 | 19.798 | 10.457 | −0.21 | −0.83 | −0.510 | 2.692 |
| ATOM | 25 | PB | UDP | 1 | 3.931 | 20.191 | 11.972 | −0.51 | +1.40 | +1.019 | 2.692 |
| ATOM | 26 | O2B | UDP | 1 | 3.166 | 19.151 | 12.707 | +0.07 | −0.29 | −0.255 | 2.692 |
| ATOM | 27 | O3B | UDP | 1 | 3.740 | 21.629 | 12.292 | −0.32 | −0.33 | −0.255 | 2.692 |
| ATOM | 28 | O1B | UDP | 1 | 5.525 | 19.965 | 12.113 | −0.28 | −0.37 | −0.255 | 2.692 |
| TER | | | | | | | | | | | |

ENDMDL

| | |
|---|---|
| MODEL | 7 |

| USER | Run = 7 | |
|---|---|---|
| USER | Cluster Rank = 1 | |
| USER | Number of conformations in this cluster = 4 | |
| USER | | |
| USER | RMSD from reference structure | = 2.524 A |
| USER | | |
| USER | Estimated Free Energy of Binding | = −9.52 kcal/mol [= (1) + (3)] |
| USER | Estimated Inhibition Constant, Ki | = +1.04e−07 [Temperature = 298.15 K] |
| USER | | |
| USER | Final Docked Energy | = −12.10 kcal/mol [= (1) + (2)] |
| USER | | |
| USER | (1) Final Intermolecular Energy | = −11.70 kcal/mol |
| USER | (2) Final Internal Energy of Ligand | = −0.40 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran −0.795355 18.167744 8.740053 | | | | | | | | | | |
| USER | NEWDPF quat0 0.963898 0.193298 0.183131 52.736620 | | | | | | | | | | |
| USER | NEWDPF ndihe 7 | | | | | | | | | | |
| USER | NEWDPF dihe −109.32 −172.52 −147.46 −10.71 −1.98 −38.43 −10.62 | | | | | | | | | | |
| USER | | | | | | | | | | | |
| USER | | | | Rank | x | y | z | vdW | Elec | q | RMS |
| ATOM | 1 | C1_{—} | UDP | 1 | −0.804 | 17.916 | 7.340 | −0.66 | +0.31 | +0.324 | 2.524 |
| ATOM | 2 | C5 | UDP | 1 | 0.271 | 21.341 | 6.309 | −0.56 | +0.00 | +0.000 | 2.524 |
| ATOM | 3 | C6 | UDP | 1 | 0.239 | 20.121 | 6.869 | −0.54 | +0.00 | +0.000 | 2.524 |
| ATOM | 4 | C2_{—} | UDP | 1 | 0.481 | 17.111 | 7.135 | −0.68 | +0.08 | +0.113 | 2.524 |
| ATOM | 5 | C3_{—} | UDP | 1 | 0.414 | 16.163 | 8.355 | −0.66 | +0.07 | +0.113 | 2.524 |
| ATOM | 6 | C4 | UDP | 1 | −0.799 | 21.753 | 5.456 | −0.65 | +0.25 | +0.396 | 2.524 |
| ATOM | 7 | O4_{—} | UDP | 1 | −0.795 | 18.168 | 8.740 | −0.07 | −0.22 | −0.227 | 2.524 |
| ATOM | 8 | O2 | UDP | 1 | −2.805 | 18.795 | 5.678 | −0.35 | −0.71 | −0.396 | 2.524 |
| ATOM | 9 | O4 | UDP | 1 | −0.916 | 22.833 | 4.878 | −0.12 | −0.27 | −0.396 | 2.524 |
| ATOM | 10 | C4_{—} | UDP | 1 | −0.268 | 17.003 | 9.433 | −0.68 | +0.10 | +0.113 | 2.524 |
| ATOM | 11 | N1 | UDP | 1 | −0.813 | 19.230 | 6.684 | −0.38 | −0.20 | −0.211 | 2.524 |
| ATOM | 12 | C2 | UDP | 1 | −1.862 | 19.565 | 5.874 | −0.78 | +0.38 | +0.396 | 2.524 |
| ATOM | 13 | N3 | UDP | 1 | −1.818 | 20.811 | 5.303 | −0.46 | −0.22 | −0.440 | 2.524 |
| ATOM | 14 | H3 | UDP | 1 | −2.612 | 21.078 | 4.703 | −0.26 | −0.17 | +0.440 | 2.524 |
| ATOM | 15 | O2_{—} | UDP | 1 | 0.412 | 16.365 | 5.943 | −0.25 | −0.33 | −0.537 | 2.524 |
| ATOM | 16 | HO2_{—} | UDP | 1 | 0.793 | 15.422 | 6.105 | −0.07 | +0.01 | +0.424 | 2.524 |
| ATOM | 17 | O3_{—} | UDP | 1 | −0.433 | 15.070 | 7.989 | −0.21 | −0.23 | −0.537 | 2.524 |
| ATOM | 18 | HO3_{—} | UDP | 1 | 0.086 | 14.421 | 7.382 | −0.37 | −0.13 | +0.424 | 2.524 |
| ATOM | 19 | C5_{—} | UDP | 1 | 0.696 | 17.479 | 10.519 | −0.43 | +0.11 | +0.113 | 2.524 |
| ATOM | 20 | O5_{—} | UDP | 1 | 1.856 | 17.980 | 9.883 | −0.01 | −0.41 | −0.368 | 2.524 |
| ATOM | 21 | PA | UDP | 1 | 3.212 | 18.583 | 10.420 | −0.53 | +1.47 | +1.019 | 2.524 |
| ATOM | 22 | O1A | UDP | 1 | 3.849 | 17.558 | 11.294 | −0.34 | −0.49 | −0.255 | 2.524 |
| ATOM | 23 | O2A | UDP | 1 | 3.994 | 19.107 | 9.257 | −0.41 | −0.50 | −0.255 | 2.524 |
| ATOM | 24 | O3A | UDP | 1 | 2.534 | 19.712 | 11.318 | +0.00 | −0.62 | −0.510 | 2.524 |
| ATOM | 25 | PB | UDP | 1 | 2.979 | 20.452 | 12.665 | −0.51 | +1.13 | +1.019 | 2.524 |
| ATOM | 26 | O2B | UDP | 1 | 3.534 | 19.314 | 13.441 | +0.09 | −0.27 | −0.255 | 2.524 |
| ATOM | 27 | O3B | UDP | 1 | 1.937 | 21.328 | 13.260 | −0.03 | −0.26 | −0.255 | 2.524 |
| ATOM | 28 | O1B | UDP | 1 | 4.140 | 21.478 | 12.205 | −0.29 | −0.38 | −0.255 | 2.524 |
| TER | | | | | | | | | | | |

ENDMDL

| | |
|---|---|
| MODEL | 10 |

| USER | Run = 10 | |
|---|---|---|
| USER | Cluster Rank = 2 | |
| USER | Number of conformations in this cluster = 4 | |
| USER | | |
| USER | RMSD from reference structure | = 2.584 A |
| USER | | |
| USER | Estimated Free Energy of Binding | = −8.95 kcal/mol [= (1) + (3)] |
| USER | Estimated Inhibition Constant, Ki | = +2.77e−07 [Temperature = 298.15 K] |
| USER | | |
| USER | Final Docked Energy | = −11.99 kcal/mol [= (1) + (2)] |
| USER | | |
| USER | (1) Final Intermolecular Energy | = −11.12 kcal/mol |
| USER | (2) Final Internal Energy of Ligand | = −0.87 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran −0.180274 18.566620 9.252676 | | | | | | | | | | |
| USER | NEWDPF quat0 −0.838918 −0.544110 −0.012656 −50.768828 | | | | | | | | | | |
| USER | NEWDPF ndihe 7 | | | | | | | | | | |
| USER | NEWDPF dihe −87.11 −161.05 −27.98 155.46 −165.28 −35.08 −179.90 | | | | | | | | | | |
| USER | | | | | | | | | | | |
| USER | | | | Rank | x | y | z | vdW | Elec | q | RMS |
| ATOM | 1 | C1_{—} | UDP | 2 | −0.618 | 18.089 | 7.986 | −0.61 | +0.31 | +0.324 | 2.584 |
| ATOM | 2 | C5 | UDP | 2 | −0.011 | 21.271 | 6.126 | −0.60 | +0.00 | +0.000 | 2.584 |
| ATOM | 3 | C6 | UDP | 2 | 0.166 | 20.160 | 6.860 | −0.55 | +0.00 | +0.000 | 2.584 |
| ATOM | 4 | C2_{—} | UDP | 2 | 0.561 | 17.230 | 7.525 | −0.64 | +0.09 | +0.113 | 2.584 |
| ATOM | 5 | C3_{—} | UDP | 2 | 0.902 | 16.495 | 8.843 | −0.66 | +0.08 | +0.113 | 2.584 |
| ATOM | 6 | C4 | UDP | 2 | −1.304 | 21.566 | 5.594 | −0.72 | +0.24 | +0.396 | 2.584 |
| ATOM | 7 | O4_{—} | UDP | 2 | −0.180 | 18.567 | 9.253 | −0.05 | −0.22 | −0.227 | 2.584 |
| ATOM | 8 | O2 | UDP | 2 | −3.060 | 18.735 | 6.910 | −0.39 | −0.88 | −0.396 | 2.584 |
| ATOM | 9 | O4 | UDP | 2 | −1.624 | 22.539 | 4.914 | −0.16 | −0.20 | −0.396 | 2.584 |
| ATOM | 10 | C4_{—} | UDP | 2 | 0.568 | 17.518 | 9.927 | −0.57 | +0.11 | +0.113 | 2.584 |
| ATOM | 11 | N1 | UDP | 2 | −0.866 | 19.278 | 7.160 | −0.37 | −0.20 | −0.211 | 2.584 |
| ATOM | 12 | C2 | UDP | 2 | −2.124 | 19.503 | 6.674 | −0.82 | +0.46 | +0.396 | 2.584 |
| ATOM | 13 | N3 | UDP | 2 | −2.294 | 20.638 | 5.923 | −0.53 | −0.28 | −0.440 | 2.584 |
| ATOM | 14 | H3 | UDP | 2 | −3.242 | 20.823 | 5.565 | −0.28 | +0.02 | +0.440 | 2.584 |
| ATOM | 15 | O2_{—} | UDP | 2 | 0.144 | 16.300 | 6.553 | −0.27 | −0.31 | −0.537 | 2.584 |
| ATOM | 16 | HO2_{—} | UDP | 2 | 0.256 | 15.344 | 6.920 | −0.12 | +0.01 | +0.424 | 2.584 |
| ATOM | 17 | O3_{—} | UDP | 2 | 0.013 | 15.380 | 8.943 | −0.32 | −0.35 | −0.537 | 2.584 |
| ATOM | 18 | HO3_{—} | UDP | 2 | 0.432 | 14.564 | 8.475 | −0.32 | +0.12 | +0.424 | 2.584 |
| ATOM | 19 | C5_{—} | UDP | 2 | 1.809 | 18.140 | 10.566 | −0.49 | +0.13 | +0.113 | 2.584 |
| ATOM | 20 | O5_{—} | UDP | 2 | 1.743 | 17.911 | 11.960 | +0.01 | −0.44 | −0.368 | 2.584 |
| ATOM | 21 | PA | UDP | 2 | 2.639 | 18.367 | 13.177 | −0.33 | +1.14 | +1.019 | 2.584 |
| ATOM | 22 | O1A | UDP | 2 | 3.444 | 17.191 | 13.613 | +0.02 | −0.28 | −0.255 | 2.584 |
| ATOM | 23 | O2A | UDP | 2 | 3.363 | 19.618 | 12.793 | +0.04 | −0.29 | −0.255 | 2.584 |
| ATOM | 24 | O3A | UDP | 2 | 1.425 | 18.594 | 14.185 | −0.16 | −0.60 | −0.510 | 2.584 |
| ATOM | 25 | PB | UDP | 2 | 0.187 | 19.608 | 14.196 | −0.59 | +1.21 | +1.019 | 2.584 |
| ATOM | 26 | O2B | UDP | 2 | −0.715 | 18.961 | 13.209 | −0.43 | −0.45 | −0.255 | 2.584 |
| ATOM | 27 | O3B | UDP | 2 | 0.561 | 21.038 | 14.045 | −0.07 | −0.25 | −0.255 | 2.584 |
| ATOM | 28 | O1B | UDP | 2 | −0.408 | 19.513 | 15.695 | −0.16 | −0.17 | −0.255 | 2.584 |
| TER | | | | | | | | | | | |

ENDMDL

| | |
|---|---|
| MODEL | 4 |

| USER | Run = 4 | |
|---|---|---|
| USER | Cluster Rank = 2 | |
| USER | Number of conformations in this cluster = 4 | |
| USER | | |
| USER | RMSD from reference structure | = 2.673 A |
| USER | | |
| USER | Estimated Free Energy of Binding | = −9.07 kcal/mol [= (1) + (3)] |
| USER | Estimated Inhibition Constant, Ki | = +2.24e−07 [Temperature = 298.15 K] |
| USER | | |
| USER | Final Docked Energy | = −11.96 kcal/mol [= (1) + (2)] |
| USER | | |
| USER | (1) Final Intermolecular Energy | = −11.25 kcal/mol |
| USER | (2) Final Internal Energy of Ligand | = −0.71 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran −0.136219 18.367965 9.164478 | | | | | | | | | | |
| USER | NEWDPF quat0 0.856724 0.515677 −0.010049 54.901151 | | | | | | | | | | |
| USER | NEWDPF ndihe 7 | | | | | | | | | | |
| USER | NEWDPF dihe 180.00 170.97 −156.14 102.29 104.63 −38.50 −180.00 | | | | | | | | | | |
| USER | | | | | | | | | | | |
| USER | | | | Rank | x | y | z | vdW | Elec | q | RMS |
| ATOM | 1 | C1_{—} | UDP | 2 | −0.617 | 17.995 | 7.879 | −0.62 | +0.31 | +0.324 | 2.673 |
| ATOM | 2 | C5 | UDP | 2 | 0.051 | 21.281 | 6.232 | −0.59 | +0.00 | +0.000 | 2.673 |
| ATOM | 3 | C6 | UDP | 2 | 0.208 | 20.116 | 6.881 | −0.54 | +0.00 | +0.000 | 2.673 |
| ATOM | 4 | C2_{—} | UDP | 2 | 0.523 | 17.135 | 7.330 | −0.66 | +0.08 | +0.113 | 2.673 |
| ATOM | 5 | C3_{—} | UDP | 2 | 0.870 | 16.299 | 8.584 | −0.65 | +0.07 | +0.113 | 2.673 |
| ATOM | 6 | C4 | UDP | 2 | −1.243 | 21.651 | 5.754 | −0.73 | +0.25 | +0.396 | 2.673 |
| ATOM | 7 | O4_{—} | UDP | 2 | −0.136 | 18.368 | 9.164 | −0.04 | −0.22 | −0.227 | 2.673 |
| ATOM | 8 | O2 | UDP | 2 | −3.060 | 18.790 | 6.910 | −0.41 | −0.86 | −0.396 | 2.673 |
| ATOM | 9 | O4 | UDP | 2 | −1.547 | 22.680 | 5.151 | −0.15 | −0.23 | −0.396 | 2.673 |
| ATOM | 10 | C4_{—} | UDP | 2 | 0.593 | 17.252 | 9.746 | −0.59 | +0.10 | +0.113 | 2.673 |
| ATOM | 11 | N1 | UDP | 2 | −0.845 | 19.247 | 7.144 | −0.37 | −0.20 | −0.211 | 2.673 |
| ATOM | 12 | C2 | UDP | 2 | −2.105 | 19.544 | 6.706 | −0.82 | +0.45 | +0.396 | 2.673 |
| ATOM | 13 | N3 | UDP | 2 | −2.255 | 20.733 | 6.040 | −0.55 | −0.29 | −0.440 | 2.673 |
| ATOM | 14 | H3 | UDP | 2 | −3.205 | 20.972 | 5.720 | −0.28 | +0.07 | +0.440 | 2.673 |
| ATOM | 15 | O2_{—} | UDP | 2 | 0.056 | 16.290 | 6.305 | −0.27 | −0.33 | −0.537 | 2.673 |
| ATOM | 16 | HO2_{—} | UDP | 2 | 0.823 | 16.090 | 5.647 | +0.01 | +0.23 | +0.424 | 2.673 |
| ATOM | 17 | O3_{—} | UDP | 2 | −0.052 | 15.207 | 8.627 | −0.36 | −0.30 | −0.537 | 2.673 |
| ATOM | 18 | HO3_{—} | UDP | 2 | 0.057 | 14.632 | 7.780 | −0.35 | +0.00 | +0.424 | 2.673 |
| ATOM | 19 | C5_{—} | UDP | 2 | 1.867 | 17.789 | 10.396 | −0.51 | +0.13 | +0.113 | 2.673 |
| ATOM | 20 | O5_{—} | UDP | 2 | 1.823 | 17.466 | 11.772 | +0.04 | −0.41 | −0.368 | 2.673 |
| ATOM | 21 | PA | UDP | 2 | 2.794 | 17.750 | 12.983 | −0.27 | +1.15 | +1.019 | 2.673 |
| ATOM | 22 | O1A | UDP | 2 | 2.039 | 17.490 | 14.242 | −0.01 | −0.27 | −0.255 | 2.673 |
| ATOM | 23 | O2A | UDP | 2 | 4.070 | 17.003 | 12.757 | −0.28 | −0.47 | −0.255 | 2.673 |
| ATOM | 24 | O3A | UDP | 2 | 2.899 | 19.321 | 12.732 | +0.06 | −0.58 | −0.510 | 2.673 |
| ATOM | 25 | PB | UDP | 2 | 4.116 | 20.290 | 12.360 | −0.51 | +1.31 | +1.019 | 2.673 |
| ATOM | 26 | O2B | UDP | 2 | 5.116 | 19.890 | 13.384 | +0.01 | −0.26 | −0.255 | 2.673 |
| ATOM | 27 | O3B | UDP | 2 | 3.740 | 21.718 | 12.198 | −0.32 | −0.34 | −0.255 | 2.673 |
| ATOM | 28 | O1B | UDP | 2 | 4.569 | 19.831 | 10.879 | −0.43 | −0.47 | −0.255 | 2.673 |
| TER | | | | | | | | | | | |

ENDMDL

| | |
|---|---|
| MODEL | 8 |

| USER | Run = 8 | |
|---|---|---|
| USER | Cluster Rank = 2 | |
| USER | Number of conformations in this cluster = 4 | |
| USER | | |
| USER | RMSD from reference structure | = 2.584 A |
| USER | | |
| USER | Estimated Free Energy of Binding | = −8.75 kcal/mol [= (1) + (3)] |
| USER | Estimated Inhibition Constant, Ki | = +3.87e−07 [Temperature = 298.15 K] |
| USER | | |
| USER | Final Docked Energy | = −11.83 kcal/mol [= (1) + (2)] |
| USER | | |
| USER | (1) Final Intermolecular Energy | = −10.93 kcal/mol |
| USER | (2) Final Internal Energy of Ligand | = −0.91 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran −0.022664 18.446691 9.057004 | | | | | | | | | | |
| USER | NEWDPF quat0 0.767388 0.618852 −0.167745 57.739009 | | | | | | | | | | |
| USER | NEWDPF ndihe 7 | | | | | | | | | | |
| USER | NEWDPF dihe −58.71 178.16 19.94 37.66 155.46 23.04 178.89 | | | | | | | | | | |
| USER | | | | | | | | | | | |
| USER | | | | Rank | x | y | z | vdW | Elec | q | RMS |
| ATOM | 1 | C1_{—} | UDP | 2 | −0.747 | 18.034 | 7.905 | −0.62 | +0.31 | +0.324 | 2.584 |
| ATOM | 2 | C5 | UDP | 2 | −0.154 | 21.145 | 5.923 | −0.59 | +0.00 | +0.000 | 2.584 |
| ATOM | 3 | C6 | UDP | 2 | 0.034 | 20.014 | 6.622 | −0.56 | +0.00 | +0.000 | 2.584 |
| ATOM | 4 | C2_{—} | UDP | 2 | 0.216 | 17.051 | 7.236 | −0.70 | +0.08 | +0.113 | 2.584 |
| ATOM | 5 | C3_{—} | UDP | 2 | 0.720 | 16.270 | 8.472 | −0.66 | +0.07 | +0.113 | 2.584 |
| ATOM | 6 | C4 | UDP | 2 | −1.483 | 21.588 | 5.646 | −0.74 | +0.23 | +0.396 | 2.584 |
| ATOM | 7 | O4_{—} | UDP | 2 | −0.023 | 18.447 | 9.057 | −0.03 | −0.22 | −0.227 | 2.584 |
| ATOM | 8 | O2 | UDP | 2 | −3.262 | 18.963 | 7.310 | −0.41 | −0.79 | −0.396 | 2.584 |
| ATOM | 9 | O4 | UDP | 2 | −1.816 | 22.599 | 5.027 | −0.16 | −0.20 | −0.396 | 2.584 |
| ATOM | 10 | C4_{—} | UDP | 2 | 0.718 | 17.314 | 9.588 | −0.59 | +0.10 | +0.113 | 2.584 |
| ATOM | 11 | N1 | UDP | 2 | −1.014 | 19.251 | 7.127 | −0.38 | −0.21 | −0.211 | 2.584 |
| ATOM | 12 | C2 | UDP | 2 | −2.308 | 19.622 | 6.889 | −0.84 | +0.47 | +0.396 | 2.584 |
| ATOM | 13 | N3 | UDP | 2 | −2.490 | 20.776 | 6.170 | −0.56 | −0.29 | −0.440 | 2.584 |
| ATOM | 14 | H3 | UDP | 2 | −3.462 | 21.070 | 5.999 | −0.27 | +0.11 | +0.440 | 2.584 |
| ATOM | 15 | O2_{—} | UDP | 2 | −0.482 | 16.184 | 6.373 | −0.24 | −0.36 | −0.537 | 2.584 |
| ATOM | 16 | HO2_{—} | UDP | 2 | −0.823 | 15.371 | 6.905 | −0.04 | +0.20 | +0.424 | 2.584 |
| ATOM | 17 | O3_{—} | UDP | 2 | −0.254 | 15.262 | 8.755 | −0.37 | −0.35 | −0.537 | 2.584 |
| ATOM | 18 | HO3_{—} | UDP | 2 | −0.250 | 14.560 | 8.002 | −0.31 | +0.13 | +0.424 | 2.584 |
| ATOM | 19 | C5_{—} | UDP | 2 | 2.122 | 17.785 | 9.968 | −0.52 | +0.13 | +0.113 | 2.584 |
| ATOM | 20 | O5_{—} | UDP | 2 | 2.289 | 17.578 | 11.357 | −0.02 | −0.43 | −0.368 | 2.584 |
| ATOM | 21 | PA | UDP | 2 | 2.538 | 18.549 | 12.576 | −0.37 | +1.19 | +1.019 | 2.584 |
| ATOM | 22 | O1A | UDP | 2 | 3.440 | 17.852 | 13.536 | +0.07 | −0.29 | −0.255 | 2.584 |
| ATOM | 23 | O2A | UDP | 2 | 2.964 | 19.881 | 12.044 | −0.01 | −0.31 | −0.255 | 2.584 |
| ATOM | 24 | O3A | UDP | 2 | 1.038 | 18.507 | 13.115 | −0.13 | −0.78 | −0.510 | 2.584 |
| ATOM | 25 | PB | UDP | 2 | 0.310 | 19.172 | 14.375 | −0.53 | +1.25 | +1.019 | 2.584 |
| ATOM | 26 | O2B | UDP | 2 | −0.818 | 19.873 | 13.710 | −0.29 | −0.43 | −0.255 | 2.584 |
| ATOM | 27 | O3B | UDP | 2 | 1.217 | 19.910 | 15.292 | +0.01 | −0.18 | −0.255 | 2.584 |
| ATOM | 28 | O1B | UDP | 2 | −0.208 | 17.921 | 15.257 | −0.22 | −0.32 | −0.255 | 2.584 |
| TER | | | | | | | | | | | |

ENDMDL

| | |
|---|---|
| MODEL | 1 |

| USER | Run = 1 | |
|---|---|---|
| USER | Cluster Rank = 2 | |
| USER | Number of conformations in this cluster = 4 | |
| USER | | |
| USER | RMSD from reference structure | = 2.605 A |
| USER | | |
| USER | Estimated Free Energy of Binding | = −8.68 kcal/mol [= (1) + (3)] |
| USER | Estimated Inhibition Constant, Ki | = +4.35e−07 [Temperature = 298.15 K] |
| USER | | |
| USER | Final Docked Energy | = −11.56 kcal/mol [= (1) + (2)] |
| USER | | |
| USER | (1) Final Intermolecular Energy | = −10.86 kcal/mol |
| USER | (2) Final Internal Energy of Ligand | = −0.71 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran 0.159626 18.707810 8.915643 | | | | | | | | | | |
| USER | NEWDPF quat0 0.740916 0.642215 −0.196480 53.848304 | | | | | | | | | | |
| USER | NEWDPF ndihe 7 | | | | | | | | | | |
| USER | NEWDPF dihe −146.03 −167.95 149.21 −79.10 −145.02 −41.30 149.16 | | | | | | | | | | |
| USER | | | | | | | | | | | |
| USER | | | | Rank | x | y | z | vdW | Elec | q | RMS |
| ATOM | 1 | C1_{—} | UDP | 2 | −0.549 | 18.202 | 7.791 | −0.60 | +0.31 | +0.324 | 2.605 |
| ATOM | 2 | C5 | UDP | 2 | 0.058 | 21.149 | 5.577 | −0.51 | +0.00 | +0.000 | 2.605 |
| ATOM | 3 | C6 | UDP | 2 | 0.241 | 20.078 | 6.365 | −0.54 | +0.00 | +0.000 | 2.605 |
| ATOM | 4 | C2_{—} | UDP | 2 | 0.425 | 17.173 | 7.214 | −0.67 | +0.08 | +0.113 | 2.605 |
| ATOM | 5 | C3_{—} | UDP | 2 | 0.916 | 16.495 | 8.514 | −0.64 | +0.08 | +0.113 | 2.605 |
| ATOM | 6 | C4 | UDP | 2 | −1.270 | 21.564 | 5.250 | −0.66 | +0.21 | +0.396 | 2.605 |
| ATOM | 7 | O4_{—} | UDP | 2 | 0.160 | 18.708 | 8.916 | −0.02 | −0.22 | −0.227 | 2.605 |
| ATOM | 8 | O2 | UDP | 2 | −3.060 | 19.071 | 7.095 | −0.50 | −0.74 | −0.396 | 2.605 |
| ATOM | 9 | O4 | UDP | 2 | −1.598 | 22.521 | 4.549 | −0.14 | −0.18 | −0.396 | 2.605 |
| ATOM | 10 | C4_{—} | UDP | 2 | 0.898 | 17.623 | 9.543 | −0.56 | +0.11 | +0.113 | 2.605 |
| ATOM | 11 | N1 | UDP | 2 | −0.811 | 19.353 | 6.916 | −0.37 | −0.20 | −0.211 | 2.605 |
| ATOM | 12 | C2 | UDP | 2 | −2.103 | 19.699 | 6.635 | −0.82 | +0.43 | +0.396 | 2.605 |
| ATOM | 13 | N3 | UDP | 2 | −2.280 | 20.791 | 5.825 | −0.51 | −0.25 | −0.440 | 2.605 |
| ATOM | 14 | H3 | UDP | 2 | −3.251 | 21.067 | 5.620 | −0.27 | −0.03 | +0.440 | 2.605 |
| ATOM | 15 | O2_{—} | UDP | 2 | −0.260 | 16.239 | 6.414 | −0.27 | −0.34 | −0.537 | 2.605 |
| ATOM | 16 | HO2_{—} | UDP | 2 | 0.393 | 15.502 | 6.110 | −0.04 | +0.11 | +0.424 | 2.605 |
| ATOM | 17 | O3_{—} | UDP | 2 | −0.058 | 15.508 | 8.864 | −0.31 | −0.35 | −0.537 | 2.605 |
| ATOM | 18 | HO3_{—} | UDP | 2 | 0.110 | 14.651 | 8.318 | −0.31 | +0.15 | +0.424 | 2.605 |
| ATOM | 19 | C5_{—} | UDP | 2 | 2.295 | 18.129 | 9.902 | −0.51 | +0.14 | +0.113 | 2.605 |
| ATOM | 20 | O5_{—} | UDP | 2 | 2.237 | 18.683 | 11.202 | +0.02 | −0.44 | −0.368 | 2.605 |
| ATOM | 21 | PA | UDP | 2 | 3.316 | 19.335 | 12.151 | −0.45 | +1.26 | +1.019 | 2.605 |
| ATOM | 22 | O1A | UDP | 2 | 3.995 | 18.233 | 12.890 | +0.03 | −0.34 | −0.255 | 2.605 |
| ATOM | 23 | O2A | UDP | 2 | 4.160 | 20.269 | 11.342 | −0.43 | −0.42 | −0.255 | 2.605 |
| ATOM | 24 | O3A | UDP | 2 | 2.265 | 20.059 | 13.106 | +0.04 | −0.54 | −0.510 | 2.605 |
| ATOM | 25 | PB | UDP | 2 | 1.096 | 19.518 | 14.055 | −0.47 | +1.10 | +1.019 | 2.605 |
| ATOM | 26 | O2B | UDP | 2 | −0.103 | 19.862 | 13.248 | −0.18 | −0.34 | −0.255 | 2.605 |
| ATOM | 27 | 03B | UDP | 2 | 1.200 | 19.957 | 15.470 | +0.01 | −0.16 | −0.255 | 2.605 |
| ATOM | 28 | O1B | UDP | 2 | 1.327 | 17.920 | 14.120 | −0.22 | −0.36 | −0.255 | 2.605 |
| TER | | | | | | | | | | | |

ENDMDL

| | |
|---|---|
| MODEL | 2 |

| USER | Run = 2 | |
|---|---|---|
| USER | Cluster Rank = 3 | |
| USER | Number of conformations in this cluster = 1 | |
| USER | | |
| USER | RMSD from reference structure | = 4.338 A |
| USER | | |
| USER | Estimated Free Energy of Binding | = −7.33 kcal/mol [= (1) + (3)] |
| USER | Estimated Inhibition Constant, Ki | = +4.26e−06 [Temperature = 298.15 K] |
| USER | | |
| USER | Final Docked Energy | = −10.51 kcal/mol [= (1) + (2)] |
| USER | | |
| USER | (1) Final Intermolecular Energy | = −9.51 kcal/mol |
| USER | (2) Final Internal Energy of Ligand | = −1.01 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran 2.065615 17.873441 12.053676 | | | | | | | | | | |
| USER | NEWDPF quat0 −0.053248 −0.487642 −0.871418 −92.317993 | | | | | | | | | | |
| USER | NEWDPF ndihe 7 | | | | | | | | | | |
| USER | NEWDPF dihe −61.40 −99.56 −177.90 −53.55 163.72 47.50 140.73 | | | | | | | | | | |
| USER | | | | | | | | | | | |
| USER | | | | Rank | x | y | z | vdW | Elec | q | RMS |
| ATOM | 1 | C1_{—} | UDP | 3 | 2.754 | 17.400 | 10.903 | −0.50 | +0.43 | +0.324 | 4.338 |
| ATOM | 2 | C5 | UDP | 3 | 0.042 | 18.189 | 8.459 | −0.55 | +0.00 | +0.000 | 4.338 |
| ATOM | 3 | C6 | UDP | 3 | 0.996 | 18.302 | 9.397 | −0.52 | +0.00 | +0.000 | 4.338 |
| ATOM | 4 | C2_{—} | UDP | 3 | 3.758 | 18.521 | 10.626 | −0.52 | +0.18 | +0.113 | 4.338 |
| ATOM | 5 | C3_{—} | UDP | 3 | 4.239 | 18.825 | 12.064 | −0.42 | +0.16 | +0.113 | 4.338 |
| ATOM | 6 | C4 | UDP | 3 | −0.221 | 16.913 | 7.872 | −0.69 | +0.31 | +0.396 | 4.338 |
| ATOM | 7 | O4_{—} | UDP | 3 | 2.066 | 17.873 | 12.054 | +0.02 | −0.27 | −0.227 | 4.338 |
| ATOM | 8 | O2 | UDP | 3 | 2.184 | 14.995 | 9.706 | −0.28 | −0.24 | −0.396 | 4.338 |
| ATOM | 9 | O4 | UDP | 3 | −1.060 | 16.649 | 7.011 | −0.17 | −0.35 | −0.396 | 4.338 |
| ATOM | 10 | C4_{—} | UDP | 3 | 3.006 | 18.561 | 12.925 | −0.34 | +0.13 | +0.113 | 4.338 |
| ATOM | 11 | N1 | UDP | 3 | 1.737 | 17.219 | 9.858 | −0.35 | −0.22 | −0.211 | 4.338 |
| ATOM | 12 | C2 | UDP | 3 | 1.536 | 15.975 | 9.330 | −0.64 | +0.30 | +0.396 | 4.338 |
| ATOM | 13 | N3 | UDP | 3 | 0.562 | 15.870 | 8.370 | −0.43 | −0.22 | −0.440 | 4.338 |
| ATOM | 14 | H3 | UDP | 3 | 0.393 | 14.933 | 7.977 | −0.25 | −0.02 | +0.440 | 4.338 |
| ATOM | 15 | O2_{—} | UDP | 3 | 4.833 | 18.042 | 9.852 | −0.52 | −1.17 | −0.537 | 4.338 |
| ATOM | 16 | HO2_{—} | UDP | 3 | 5.576 | 17.692 | 10.474 | +0.05 | +1.03 | +0.424 | 4.338 |
| ATOM | 17 | O3_{—} | UDP | 3 | 5.257 | 17.870 | 12.375 | −0.27 | −1.08 | −0.537 | 4.338 |
| ATOM | 18 | HO3_{—} | UDP | 3 | 5.926 | 18.285 | 13.039 | +0.10 | +0.58 | +0.424 | 4.338 |
| ATOM | 19 | C5_{—} | UDP | 3 | 2.347 | 19.841 | 13.437 | −0.39 | +0.12 | +0.113 | 4.338 |
| ATOM | 20 | O5_{—} | UDP | 3 | 1.498 | 19.494 | 14.513 | +0.04 | −0.35 | −0.368 | 4.338 |
| ATOM | 21 | PA | UDP | 3 | −0.034 | 19.127 | 14.610 | −0.56 | +1.26 | +1.019 | 4.338 |
| ATOM | 22 | O1A | UDP | 3 | −0.601 | 19.849 | 15.784 | −0.13 | −0.14 | −0.255 | 4.338 |
| ATOM | 23 | O2A | UDP | 3 | −0.663 | 19.350 | 13.271 | −0.45 | −0.42 | −0.255 | 4.338 |
| ATOM | 24 | O3A | UDP | 3 | 0.199 | 17.594 | 14.979 | −0.16 | −0.77 | −0.510 | 4.338 |
| ATOM | 25 | PB | UDP | 3 | 0.427 | 16.824 | 16.363 | −0.62 | +0.92 | +1.019 | 4.338 |
| ATOM | 26 | O2B | UDP | 3 | 1.807 | 17.259 | 16.702 | +0.02 | −0.19 | −0.255 | 4.338 |
| ATOM | 27 | O3B | UDP | 3 | 0.083 | 15.380 | 16.330 | −0.35 | −0.32 | −0.255 | 4.338 |
| ATOM | 28 | O1B | UDP | 3 | −0.660 | 17.465 | 17.373 | −0.14 | −0.15 | −0.255 | 4.338 |
| TER | | | | | | | | | | | |

ENDMDL

| | |
|---|---|
| MODEL | 9 |

| USER | Run = 9 | |
|---|---|---|
| USER | Cluster Rank = 4 | |
| USER | Number of conformations in this cluster = 1 | |
| USER | | |
| USER | RMSD from reference structure | = 4.450 A |
| USER | | |
| USER | Estimated Free Energy of Binding | = −7.39 kcal/mol [= (1) + (3)] |
| USER | Estimated Inhibition Constant, Ki | = +3.80e−06 [Temperature = 298.15 K] |
| USER | | |
| USER | Final Docked Energy | = −10.09 kcal/mol [= (1) + (2)] |
| USER | | |
| USER | (1) Final Intermolecular Energy | = −9.57 kcal/mol |
| USER | (2) Final Internal Energy of Ligand | = −0.51 kcal/mol |
| USER | (3) Torsional Free Energy | = +2.18 kcal/mol |
| USER | | |
| USER | | |

| USER | DPF = upp_n.dpf | | | | | | | | | | |
|---|---|---|---|---|---|---|---|---|---|---|---|
| USER | NEWDPF move udp_n.pdbq | | | | | | | | | | |
| USER | NEWDPF about −2.429000 18.545000 9.631000 | | | | | | | | | | |
| USER | NEWDPF tran 2.419607 19.492911 10.414915 | | | | | | | | | | |
| USER | NEWDPF quat0 0.417319 −0.864199 0.281079 −169.015093 | | | | | | | | | | |
| USER | NEWDPF ndihe 7 | | | | | | | | | | |
| USER | NEWDPF dihe 139.84 29.04 97.10 15.86 0.63 −54.79 6.36 | | | | | | | | | | |
| USER | | | | | | | | | | | |
| USER | | | | Rank | x | y | z | vdW | Elec | q | RMS |
| ATOM | 1 | C1_{—} | UDP | 4 | 2.942 | 19.101 | 11.679 | −0.45 | +0.41 | +0.324 | 4.450 |
| ATOM | 2 | C5 | UDP | 4 | −0.260 | 19.470 | 13.567 | −0.57 | +0.00 | +0.000 | 4.450 |
| ATOM | 3 | C6 | UDP | 4 | 0.669 | 19.072 | 12.684 | −0.41 | +0.00 | +0.000 | 4.450 |
| ATOM | 4 | C2_{—} | UDP | 4 | 3.049 | 17.582 | 11.542 | −0.45 | +0.16 | +0.113 | 4.450 |
| ATOM | 5 | C3_{—} | UDP | 4 | 3.622 | 17.470 | 10.110 | −0.42 | +0.21 | +0.113 | 4.450 |
| ATOM | 6 | C4 | UDP | 4 | 0.091 | 20.411 | 14.583 | −0.63 | +0.38 | +0.396 | 4.450 |
| ATOM | 7 | O4_{—} | UDP | 4 | 2.420 | 19.493 | 10.415 | −0.01 | −0.28 | −0.227 | 4.450 |
| ATOM | 8 | O2 | UDP | 4 | 3.521 | 20.896 | 13.675 | +0.00 | −0.36 | −0.396 | 4.450 |
| ATOM | 9 | O4 | UDP | 4 | −0.644 | 20.873 | 15.455 | −0.20 | −0.25 | −0.396 | 4.450 |
| ATOM | 10 | C4_{—} | UDP | 4 | 3.003 | 18.659 | 9.376 | −0.54 | +0.16 | +0.113 | 4.450 |
| ATOM | 11 | N1 | UDP | 4 | 1.973 | 19.557 | 12.685 | −0.25 | −0.24 | −0.211 | 4.450 |
| ATOM | 12 | C2 | UDP | 4 | 2.370 | 20.455 | 13.636 | −0.44 | +0.39 | +0.396 | 4.450 |
| ATOM | 13 | N3 | UDP | 4 | 1.415 | 20.851 | 14.537 | −0.29 | −0.38 | −0.440 | 4.450 |
| ATOM | 14 | H3 | UDP | 4 | 1.702 | 21.539 | 15.248 | +0.08 | +0.34 | +0.440 | 4.450 |
| ATOM | 15 | O2_{—} | UDP | 4 | 3.974 | 17.066 | 12.470 | −0.29 | −1.04 | −0.537 | 4.450 |
| ATOM | 16 | HO2_{—} | UDP | 4 | 3.476 | 16.512 | 13.181 | +0.01 | +0.48 | +0.424 | 4.450 |
| ATOM | 17 | O3_{—} | UDP | 4 | 5.037 | 17.650 | 10.210 | −0.74 | −1.30 | −0.537 | 4.450 |
| ATOM | 18 | HO3_{—} | UDP | 4 | 5.252 | 18.655 | 10.265 | +0.06 | +0.87 | +0.424 | 4.450 |
| ATOM | 19 | C5_{—} | UDP | 4 | 1.899 | 18.251 | 8.402 | −0.51 | +0.12 | +0.113 | 4.450 |
| ATOM | 20 | O5_{—} | UDP | 4 | 0.852 | 17.664 | 9.151 | −0.02 | −0.34 | −0.368 | 4.450 |
| ATOM | 21 | PA | UDP | 4 | −0.652 | 17.318 | 8.824 | −0.65 | +0.93 | +1.019 | 4.450 |
| ATOM | 22 | O1A | UDP | 4 | −0.660 | 16.351 | 7.689 | −0.18 | −0.19 | −0.255 | 4.450 |
| ATOM | 23 | O2A | UDP | 4 | −1.337 | 16.923 | 10.094 | −0.19 | −0.24 | −0.255 | 4.450 |
| ATOM | 24 | O3A | UDP | 4 | −1.034 | 18.773 | 8.297 | −0.07 | −0.52 | −0.510 | 4.450 |
| ATOM | 25 | PB | UDP | 4 | −2.171 | 19.316 | 7.311 | −0.81 | +1.25 | +1.019 | 4.450 |
| ATOM | 26 | O2B | UDP | 4 | −1.565 | 20.594 | 6.859 | −0.16 | −0.22 | −0.255 | 4.450 |
| ATOM | 27 | O3B | UDP | 4 | −3.542 | 19.297 | 7.883 | −0.42 | −0.42 | −0.255 | 4.450 |
| ATOM | 28 | O1B | UDP | 4 | −2.229 | 18.232 | 6.114 | −0.53 | −0.38 | −0.255 | 4.450 |
| TER | | | | | | | | | | | |

ENDMDL

TABLE 15
UDP
CLUSTERING HISTOGRAM

| | Lowest | | Mean | Num | |
|---|---|---|---|---|---|
| Cluster | Docked | | Docked | in | Histogram |
| Rank | Energy | Run | Energy | Clus | 5 10 15 20 25 30 35 |
| 1 | −12.66 | 17 | −12.01 | 2 | ## |
| 2 | −12.64 | 29 | −11.78 | 15 | ############### |
| 3 | −12.55 | 21 | −12.55 | 1 | # |
| 4 | −12.08 | 6 | −12.08 | 1 | # |
| 5 | −12.07 | 50 | −12.07 | 1 | # |
| 6 | −11.99 | 2 | −11.99 | 1 | # |
| 7 | −11.81 | 42 | −11.18 | 7 | ####### |
| 8 | −11.51 | 34 | −11.00 | 7 | ####### |
| 9 | −11.32 | 38 | −11.09 | 5 | ##### |
| 10 | −11.12 | 3 | −11.12 | 1 | # |
| 11 | −10.98 | 5 | −10.81 | 2 | ## |
| 12 | −10.75 | 41 | −10.75 | 1 | # |
| 13 | −10.74 | 47 | −10.74 | 1 | # |
| 14 | −10.21 | 11 | −10.21 | 1 | # |
| 15 | −10.07 | 7 | −10.07 | 1 | # |
| 16 | −9.69 | 19 | −9.69 | 1 | # |
| 17 | −9.26 | 45 | −9.26 | 1 | # |
| 18 | −7.90 | 37 | −7.90 | 1 | # |

Number of multi-member conformational clusters found = 6, out of 50 runs.

RMSD TABLE

| | Sub- | | Docked | Cluster | Reference | Grep |
|---|---|---|---|---|---|---|
| Rank | Rank | Run | Energy | RMSD | RMSD | Pattern |
| 1 | 1 | 17 | −12.66 | 0.00 | 2.59 | RANKING |
| 1 | 2 | 33 | −11.37 | 0.90 | 2.72 | RANKING |
| 2 | 1 | 29 | −12.64 | 0.00 | 1.08 | RANKING |
| 2 | 2 | 24 | −12.29 | 0.31 | 1.03 | RANKING |
| 2 | 3 | 20 | −12.24 | 0.36 | 1.09 | RANKING |
| 2 | 4 | 43 | −12.18 | 0.35 | 1.02 | RANKING |
| 2 | 5 | 23 | −12.09 | 0.51 | 1.03 | RANKING |
| 2 | 6 | 49 | −12.05 | 0.42 | 1.01 | RANKING |
| 2 | 7 | 40 | −12.01 | 0.34 | 1.08 | RANKING |
| 2 | 8 | 25 | −11.88 | 0.49 | 1.07 | RANKING |
| 2 | 9 | 46 | −11.86 | 0.48 | 0.95 | RANKING |
| 2 | 10 | 9 | −11.52 | 0.71 | 1.08 | RANKING |
| 2 | 11 | 31 | −11.29 | 0.84 | 1.02 | RANKING |
| 2 | 12 | 4 | −11.25 | 0.86 | 1.07 | RANKING |
| 2 | 13 | 15 | −11.22 | 0.86 | 1.18 | RANKING |
| 2 | 14 | 35 | −11.19 | 0.63 | 1.01 | RANKING |
| 2 | 15 | 27 | −11.01 | 0.76 | 0.99 | RANKING |
| 3 | 1 | 21 | −12.55 | 0.00 | 2.54 | RANKING |
| 4 | 1 | 6 | −12.08 | 0.00 | 2.78 | RANKING |
| 5 | 1 | 50 | −12.07 | 0.00 | 2.81 | RANKING |
| 6 | 1 | 2 | −11.99 | 0.00 | 2.09 | RANKING |
| 7 | 1 | 42 | −11.81 | 0.00 | 1.40 | RANKING |
| 7 | 2 | 16 | −11.64 | 0.52 | 1.43 | RANKING |
| 7 | 3 | 8 | −11.51 | 0.68 | 1.53 | RANKING |
| 7 | 4 | 48 | −11.17 | 0.85 | 1.59 | RANKING |
| 7 | 5 | 1 | −10.79 | 0.66 | 1.27 | RANKING |
| 7 | 6 | 44 | −10.70 | 0.73 | 1.20 | RANKING |
| 7 | 7 | 39 | −10.63 | 0.93 | 1.13 | RANKING |
| 8 | 1 | 34 | −11.51 | 0.00 | 1.40 | RANKING |
| 8 | 2 | 22 | −11.42 | 0.21 | 1.48 | RANKING |
| 8 | 3 | 26 | −11.39 | 0.63 | 1.43 | RANKING |
| 8 | 4 | 10 | −11.15 | 0.33 | 1.49 | RANKING |
| 8 | 5 | 12 | −10.92 | 0.51 | 1.21 | RANKING |
| 8 | 6 | 14 | −10.80 | 0.83 | 0.99 | RANKING |
| 8 | 7 | 36 | −9.81 | 0.88 | 0.98 | RANKING |
| 9 | 1 | 38 | −11.32 | 0.00 | 1.48 | RANKING |
| 9 | 2 | 30 | −11.26 | 0.60 | 1.17 | RANKING |
| 9 | 3 | 32 | −11.15 | 0.43 | 1.34 | RANKING |
| 9 | 4 | 18 | −10.89 | 0.68 | 1.49 | RANKING |
| 9 | 5 | 13 | −10.82 | 0.87 | 1.13 | RANKING |
| 10 | 1 | 3 | −11.12 | 0.00 | 2.42 | RANKING |
| 11 | 1 | 5 | −10.98 | 0.00 | 2.08 | RANKING |
| 11 | 2 | 28 | −10.64 | 0.44 | 2.01 | RANKING |
| 12 | 1 | 41 | −10.75 | 0.00 | 2.12 | RANKING |
| 13 | 1 | 47 | −10.74 | 0.00 | 8.31 | RANKING |
| 14 | 1 | 11 | −10.21 | 0.00 | 9.26 | RANKING |
| 15 | 1 | 7 | −10.07 | 0.00 | 8.03 | RANKING |
| 16 | 1 | 19 | −9.69 | 0.00 | 4.21 | RANKING |
| 17 | 1 | 45 | −9.26 | 0.00 | 6.47 | RANKING |
| 18 | 1 | 37 | −7.90 | 0.00 | 5.91 | RANKING |

LOWEST ENERGY DOCKED CONFORMATION from EACH CLUSTER
Residue number will be set to the conformation's cluster rank.

| | |
|---|---|
| MODEL | 17 |

| USER | Run = 17 | | | | | | | | | | |
|---|---|---|---|---|---|---|---|---|---|---|---|
| USER | Cluster Rank = 1 | | | | | | | | | | |
| USER | Number of conformations in this cluster = 2 | | | | | | | | | | |
| USER | | | | | | | | | | | |
| USER | RMSD from reference structure | | | | | = 2.593 A | | | | | |
| USER | | | | | | | | | | | |
| USER | Estimated Free Energy of Binding | | | | | = −10.07 kcal/mol [= (1) + (3)] | | | | | |
| USER | Estimated Inhibition Constant, Ki | | | | | = +4.16e−08 [Temperature = 298.15 K] | | | | | |
| USER | | | | | | | | | | | |
| USER | Final Docked Energy | | | | | = −12.66 kcal/mol [= (1) + (2)] | | | | | |
| USER | | | | | | | | | | | |
| USER | (1) Final Intermolecular Energy | | | | | = −12.56 kcal/mol | | | | | |
| USER | (2) Final Internal Energy of Ligand | | | | | = −0.10 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 1.255825 −6.309941 −7.183958 | | | | | | | | | | |
| USER | NEWDPF quat0 0.325133 0.809854 0.488287 −36.731671 | | | | | | | | | | |
| USER | NEWDPF ndihe 8 | | | | | | | | | | |
| USER | NEWDPF dihe0 67.75 91.18 45.38 60.75 −0.80 −80.84 −18.16 86.53 | | | | | | | | | | |
| USER | | | | | | | | | | | |
| USER | | | | Rank | x | y | z | vdW | Elec | q | RMS |
| ATOM | 1 | C19 | UDP | 1 | 1.012 | −7.760 | −7.629 | −0.60 | +0.08 | +0.174 | 2.593 |
| ATOM | 2 | C1 | UDP | 1 | 0.351 | −6.397 | −7.839 | −0.39 | +0.01 | +0.113 | 2.593 |
| ATOM | 3 | C3 | UDP | 1 | 1.599 | −5.509 | −8.051 | −0.58 | +0.04 | +0.113 | 2.593 |
| ATOM | 4 | O16 | UDP | 1 | 2.109 | −7.446 | −6.779 | −0.01 | −0.12 | −0.227 | 2.593 |
| ATOM | 5 | C14 | UDP | 1 | 2.661 | −6.158 | −7.165 | −0.49 | +0.07 | +0.113 | 2.593 |
| ATOM | 6 | N10 | UDP | 1 | 0.188 | −8.732 | −6.898 | −0.38 | +0.02 | +0.039 | 2.593 |
| ATOM | 7 | C11 | UDP | 1 | −0.023 | −9.969 | −7.440 | −0.76 | −0.06 | −0.073 | 2.593 |
| ATOM | 8 | O17 | UDP | 1 | 0.461 | −10.294 | −8.527 | −0.32 | −0.03 | −0.026 | 2.593 |
| ATOM | 9 | N12 | UDP | 1 | −0.789 | −10.836 | −6.702 | −0.46 | −0.24 | −0.292 | 2.593 |
| ATOM | 10 | H12 | UDP | 1 | −0.948 | −11.830 | −7.119 | +0.04 | +0.38 | +0.393 | 2.593 |
| ATOM | 11 | C9 | UDP | 1 | −1.383 | −10.565 | −5.469 | −0.52 | −0.02 | −0.023 | 2.593 |
| ATOM | 12 | O18 | UDP | 1 | −2.038 | −11.460 | −4.931 | −0.20 | −0.02 | −0.026 | 2.593 |
| ATOM | 13 | C7 | UDP | 1 | −1.131 | −9.254 | −4.960 | −0.37 | +0.00 | +0.000 | 2.593 |
| ATOM | 14 | C5 | UDP | 1 | −0.388 | −8.397 | −5.676 | −0.43 | −0.02 | −0.050 | 2.593 |
| ATOM | 15 | O21 | UDP | 1 | −0.435 | −6.403 | −9.007 | −0.05 | −0.09 | −0.537 | 2.593 |
| ATOM | 16 | H21 | UDP | 1 | −1.190 | −5.708 | −8.917 | −0.22 | −0.07 | +0.424 | 2.593 |
| ATOM | 17 | O23 | UDP | 1 | 1.976 | −5.638 | −9.426 | −0.17 | −0.34 | −0.537 | 2.593 |
| ATOM | 18 | H23 | UDP | 1 | 2.384 | −4.753 | −9.755 | +0.04 | +0.29 | +0.424 | 2.593 |
| ATOM | 19 | C25 | UDP | 1 | 2.955 | −5.356 | −5.896 | −0.44 | +0.09 | +0.113 | 2.593 |
| ATOM | 20 | O28 | UDP | 1 | 3.753 | −4.246 | −6.262 | −0.01 | −0.46 | −0.368 | 2.593 |
| ATOM | 21 | P39 | UDP | 1 | 4.410 | −3.067 | −5.444 | −0.63 | +1.83 | +1.210 | 2.593 |
| ATOM | 22 | O30 | UDP | 1 | 5.512 | −3.644 | −4.623 | −0.26 | −1.57 | −0.850 | 2.593 |
| ATOM | 23 | O31 | UDP | 1 | 4.739 | −1.956 | −6.390 | −0.23 | −1.51 | −0.850 | 2.593 |
| ATOM | 24 | O32 | UDP | 1 | 3.138 | −2.790 | −4.524 | +0.02 | −0.52 | −0.510 | 2.593 |
| ATOM | 25 | P33 | UDP | 1 | 2.158 | −1.537 | −4.363 | −0.57 | +1.47 | +1.210 | 2.593 |
| ATOM | 26 | O36 | UDP | 1 | 3.138 | −0.434 | −4.182 | −0.20 | −0.72 | −0.850 | 2.593 |
| ATOM | 27 | O35 | UDP | 1 | 1.063 | −1.736 | −3.379 | −0.28 | −0.40 | −0.255 | 2.593 |
| ATOM | 28 | O34 | UDP | 1 | 1.395 | −1.425 | −5.783 | −0.68 | −1.56 | −0.850 | 2.593 |
| TER | | | | | | | | | | | |

ENDMDL

| | |
|---|---|
| MODEL | 33 |

| USER | Run = 33 | | | | | | | | | | |
|---|---|---|---|---|---|---|---|---|---|---|---|
| USER | Cluster Rank = 1 | | | | | | | | | | |
| USER | Number of conformations in this cluster = 2 | | | | | | | | | | |
| USER | | | | | | | | | | | |
| USER | RMSD from reference structure | | | | | = 2.725 A | | | | | |
| USER | | | | | | | | | | | |
| USER | Estimated Free Energy of Binding | | | | | = −9.84 kcal/mol [= (1) + (3)] | | | | | |
| USER | Estimated Inhibition Constant, Ki | | | | | = +6.15e−08 [Temperature = 298.15 K] | | | | | |
| USER | | | | | | | | | | | |
| USER | Final Docked Energy | | | | | = −11.37 kcal/mol [= (1) + (2)] | | | | | |
| USER | | | | | | | | | | | |
| USER | (1) Final Intermolecular Energy | | | | | = −12.33 kcal/mol | | | | | |
| USER | (2) Final Internal Energy of Ligand | | | | | = +0.96 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 1.403740 −6.110069 −7.397154 | | | | | | | | | | |
| USER | NEWDPF quat0 0.233059 0.699598 0.675460 −48.506884 | | | | | | | | | | |
| USER | NEWDPF ndihe 8 | | | | | | | | | | |
| USER | NEWDPF dihe0 63.88 34.01 90.25 15.93 40.57 −52.82 18.87 −144.85 | | | | | | | | | | |
| USER | | | | | | | | | | | |
| USER | | | | Rank | x | y | z | vdW | Elec | q | RMS |
| ATOM | 1 | C19 | UDP | 1 | 0.884 | −7.439 | −7.966 | −0.62 | +0.08 | +0.174 | 2.725 |
| ATOM | 2 | C1 | UDP | 1 | 0.584 | −5.951 | −8.144 | −0.35 | +0.01 | +0.113 | 2.725 |
| ATOM | 3 | C3 | UDP | 1 | 2.019 | −5.375 | −8.167 | −0.61 | +0.06 | +0.113 | 2.725 |
| ATOM | 4 | O16 | UDP | 1 | 1.920 | −7.434 | −6.990 | +0.02 | −0.12 | −0.227 | 2.725 |
| ATOM | 5 | C14 | UDP | 1 | 2.795 | −6.297 | −7.228 | −0.42 | +0.08 | +0.113 | 2.725 |
| ATOM | 6 | N10 | UDP | 1 | −0.219 | −8.220 | −7.391 | −0.34 | +0.02 | +0.039 | 2.725 |
| ATOM | 7 | C11 | UDP | 1 | −0.148 | −9.585 | −7.402 | −0.68 | −0.05 | −0.073 | 2.725 |
| ATOM | 8 | O17 | UDP | 1 | 0.820 | −10.181 | −7.880 | −0.19 | −0.02 | −0.026 | 2.725 |
| ATOM | 9 | N12 | UDP | 1 | −1.203 | −10.257 | −6.837 | −0.38 | −0.20 | −0.292 | 2.725 |
| ATOM | 10 | H12 | UDP | 1 | −1.146 | −11.345 | −6.827 | +0.06 | +0.33 | +0.393 | 2.725 |
| ATOM | 11 | C9 | UDP | 1 | −2.342 | −9.679 | −6.276 | −0.50 | −0.02 | −0.023 | 2.725 |
| ATOM | 12 | O18 | UDP | 1 | −3.199 | −10.426 | −5.802 | −0.30 | −0.03 | −0.026 | 2.725 |
| ATOM | 13 | C7 | UDP | 1 | −2.360 | −8.250 | −6.301 | −0.46 | +0.00 | +0.000 | 2.725 |
| ATOM | 14 | C5 | UDP | 1 | −1.336 | −7.584 | −6.855 | −0.49 | −0.02 | −0.050 | 2.725 |
| ATOM | 15 | O21 | UDP | 1 | −0.045 | −5.718 | −9.382 | −0.13 | −0.09 | −0.537 | 2.725 |
| ATOM | 16 | H21 | UDP | 1 | −0.674 | −4.906 | −9.301 | −0.16 | −0.08 | +0.424 | 2.725 |
| ATOM | 17 | O23 | UDP | 1 | 2.510 | −5.528 | −9.503 | −0.17 | −0.40 | −0.537 | 2.725 |
| ATOM | 18 | H23 | UDP | 1 | 2.279 | −4.689 | −10.052 | +0.04 | +0.29 | +0.424 | 2.725 |
| ATOM | 19 | C25 | UDP | 1 | 3.119 | −5.646 | −5.882 | −0.47 | +0.09 | +0.113 | 2.725 |
| ATOM | 20 | O28 | UDP | 1 | 3.460 | −4.294 | −6.128 | +0.01 | −0.40 | −0.368 | 2.725 |
| ATOM | 21 | P39 | UDP | 1 | 4.406 | −3.263 | −5.396 | −0.61 | +1.82 | +1.210 | 2.725 |
| ATOM | 22 | O30 | UDP | 1 | 5.441 | −4.043 | −4.659 | −0.22 | −1.53 | −0.850 | 2.725 |
| ATOM | 23 | O31 | UDP | 1 | 4.862 | −2.245 | −6.391 | −0.22 | −1.68 | −0.850 | 2.725 |
| ATOM | 24 | O32 | UDP | 1 | 3.299 | −2.736 | −4.377 | +0.01 | −0.51 | −0.510 | 2.725 |
| ATOM | 25 | P33 | UDP | 1 | 1.994 | −1.831 | −4.555 | −0.53 | +1.63 | +1.210 | 2.725 |
| ATOM | 26 | O36 | UDP | 1 | 2.551 | −0.676 | −5.307 | −0.34 | −1.24 | −0.850 | 2.725 |
| ATOM | 27 | O35 | UDP | 1 | 1.224 | −1.608 | −3.303 | −0.22 | −0.34 | −0.255 | 2.725 |
| ATOM | 28 | O34 | UDP | 1 | 1.001 | −2.713 | −5.475 | −0.33 | −1.37 | −0.850 | 2.725 |
| TER | | | | | | | | | | | |

ENDMDL

| | |
|---|---|
| MODEL | 29 |

| USER | Run = 29 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.082 A | | | | | |
| USER | | | | | | | | | | | |
| USER | Estimated Free Energy of Binding | | | | | = −9.80 kcal/mol [= (1) + (3)] | | | | | |
| USER | Estimated Inhibition Constant, Ki | | | | | = +6.60e−08 [Temperature = 298.15 K] | | | | | |
| USER | | | | | | | | | | | |
| USER | Final Docked Energy | | | | | = −12.64 kcal/mol [= (1) + (2)] | | | | | |
| USER | | | | | | | | | | | |
| USER | (1) Final Intermolecular Energy | | | | | = −12.29 kcal/mol | | | | | |
| USER | (2) Final Internal Energy of Ligand | | | | | = −0.36 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.481760 −7.953557 −5.161065 | | | | | | | | | | |
| USER | NEWDPF quat0 −0.502285 −0.582314 −0.639234 −7.048210 | | | | | | | | | | |
| USER | NEWDPF ndihe 8 | | | | | | | | | | |
| USER | NEWDPF dihe0 7.79 94.48 −17.41 −37.95 4.15 −122.90 −49.04 143.57 | | | | | | | | | | |
| USER | | | | | | | | | | | |
| USER | | | | Rank | x | y | z | vdW | Elec | q | RMS |
| ATOM | 1 | C19 | UDP | 2 | −1.508 | −9.297 | −5.905 | −0.45 | +0.10 | +0.174 | 1.082 |
| ATOM | 2 | C1 | UDP | 2 | −2.525 | −8.345 | −5.277 | −0.36 | +0.08 | +0.113 | 1.082 |
| ATOM | 3 | C3 | UDP | 2 | −1.960 | −6.983 | −5.744 | −0.38 | +0.04 | +0.113 | 1.082 |
| ATOM | 4 | O16 | UDP | 2 | −0.270 | −8.645 | −5.646 | +0.02 | −0.11 | −0.227 | 1.082 |
| ATOM | 5 | C14 | UDP | 2 | −0.449 | −7.209 | −5.775 | −0.36 | +0.03 | +0.113 | 1.082 |
| ATOM | 6 | N10 | UDP | 2 | −1.424 | −10.613 | −5.257 | −0.31 | +0.03 | +0.039 | 1.082 |
| ATOM | 7 | C11 | UDP | 2 | −1.440 | −11.740 | −6.031 | −0.65 | −0.06 | −0.073 | 1.082 |
| ATOM | 8 | O17 | UDP | 2 | −1.522 | −11.684 | −7.260 | −0.25 | −0.02 | −0.026 | 1.082 |
| ATOM | 9 | N12 | UDP | 2 | −1.344 | −12.938 | −5.368 | −0.42 | −0.20 | −0.292 | 1.082 |
| ATOM | 10 | H12 | UDP | 2 | −1.340 | −13.840 | −5.978 | −0.19 | +0.10 | +0.393 | 1.082 |
| ATOM | 11 | C9 | UDP | 2 | −1.252 | −13.109 | −3.986 | −0.70 | −0.02 | −0.023 | 1.082 |
| ATOM | 12 | O18 | UDP | 2 | −1.166 | −14.257 | −3.548 | −0.21 | −0.02 | −0.026 | 1.082 |
| ATOM | 13 | C7 | UDP | 2 | −1.247 | −11.894 | −3.233 | −0.51 | +0.00 | +0.000 | 1.082 |
| ATOM | 14 | C5 | UDP | 2 | −1.345 | −10.719 | −3.872 | −0.26 | −0.03 | −0.050 | 1.082 |
| ATOM | 15 | O21 | UDP | 2 | −3.802 | −8.548 | −5.835 | −0.16 | −0.66 | −0.537 | 1.082 |
| ATOM | 16 | H21 | UDP | 2 | −4.330 | −9.214 | −5.252 | +0.07 | +0.47 | +0.424 | 1.082 |
| ATOM | 17 | O23 | UDP | 2 | −2.440 | −6.763 | −7.075 | −0.27 | −0.26 | −0.537 | 1.082 |
| ATOM | 18 | H23 | UDP | 2 | −2.527 | −5.753 | −7.246 | −0.11 | −0.13 | +0.424 | 1.082 |
| ATOM | 19 | C25 | UDP | 2 | 0.283 | −6.523 | −4.621 | −0.30 | +0.04 | +0.113 | 1.082 |
| ATOM | 20 | O28 | UDP | 2 | 0.976 | −5.408 | −5.151 | +0.09 | −0.13 | −0.368 | 1.082 |
| ATOM | 21 | P39 | UDP | 2 | 1.209 | −3.932 | −4.640 | −0.28 | +1.08 | +1.210 | 1.082 |
| ATOM | 22 | O30 | UDP | 2 | 0.041 | −3.557 | −3.794 | −0.24 | −1.35 | −0.850 | 1.082 |
| ATOM | 23 | O31 | UDP | 2 | 2.575 | −3.846 | −4.039 | +0.09 | −0.72 | −0.850 | 1.082 |
| ATOM | 24 | O32 | UDP | 2 | 1.078 | −3.251 | −6.075 | −0.11 | −0.39 | −0.510 | 1.082 |
| ATOM | 25 | P33 | UDP | 2 | 1.197 | −1.740 | −6.582 | −0.50 | +1.69 | +1.210 | 1.082 |
| ATOM | 26 | O36 | UDP | 2 | 0.284 | −1.051 | −5.632 | −0.67 | −2.36 | −0.850 | 1.082 |
| ATOM | 27 | O35 | UDP | 2 | 1.022 | −1.566 | −8.047 | −0.36 | −0.28 | −0.255 | 1.082 |
| ATOM | 28 | O34 | UDP | 2 | 2.739 | −1.337 | −6.320 | −0.19 | −1.21 | −0.850 | 1.082 |
| TER | | | | | | | | | | | |

ENDMDL

| | |
|---|---|
| MODEL | 24 |

| USER | Run = 24 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.035 A | | | | | |
| USER | | | | | | | | | | | |
| USER | Estimated Free Energy of Binding | | | | | = −9.35 kcal/mol [= (1) + (3)] | | | | | |
| USER | Estimated Inhibition Constant, Ki | | | | | = +1.41e−07 [Temperature = 298.15 K] | | | | | |
| USER | | | | | | | | | | | |
| USER | Final Docked Energy | | | | | = −12.29 kcal/mol [= (1) + (2)] | | | | | |
| USER | | | | | | | | | | | |
| USER | (1) Final Intermolecular Energy | | | | | = −11.84 kcal/mol | | | | | |
| USER | (2) Final Internal Energy of Ligand | | | | | = −0.46 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.433060 −7.950955 −4.948938 | | | | | | | | | | |
| USER | NEWDPF quat0 0.863151 0.017361 0.504647 6.771903 | | | | | | | | | | |
| USER | NEWDPF ndihe 8 | | | | | | | | | | |
| USER | NEWDPF dihe0 35.77 90.68 −0.11 −24.12 3.52 −137.15 −51.07 −178.16 | | | | | | | | | | |
| USER | | | | | | | | | | | |
| USER | | | | Rank | x | y | z | vdW | Elec | q | RMS |
| ATOM | 1 | C19 | UDP | 2 | −1.437 | −9.262 | −5.749 | −0.44 | +0.10 | +0.174 | 1.035 |
| ATOM | 2 | C1 | UDP | 2 | −2.474 | −8.312 | −5.151 | −0.35 | +0.07 | +0.113 | 1.035 |
| ATOM | 3 | C3 | UDP | 2 | −1.850 | −6.946 | −5.521 | −0.37 | +0.04 | +0.113 | 1.035 |
| ATOM | 4 | O16 | UDP | 2 | −0.206 | −8.651 | −5.382 | +0.03 | −0.11 | −0.227 | 1.035 |
| ATOM | 5 | C14 | UDP | 2 | −0.346 | −7.206 | −5.460 | −0.35 | +0.03 | +0.113 | 1.035 |
| ATOM | 6 | N10 | UDP | 2 | −1.425 | −10.605 | −5.155 | −0.31 | +0.03 | +0.039 | 1.035 |
| ATOM | 7 | C11 | UDP | 2 | −1.426 | −11.698 | −5.976 | −0.65 | −0.06 | −0.073 | 1.035 |
| ATOM | 8 | O17 | UDP | 2 | −1.435 | −11.589 | −7.204 | −0.26 | −0.02 | −0.026 | 1.035 |
| ATOM | 9 | N12 | UDP | 2 | −1.399 | −12.924 | −5.359 | −0.42 | −0.20 | −0.292 | 1.035 |
| ATOM | 10 | H12 | UDP | 2 | −1.383 | −13.801 | −6.007 | −0.24 | +0.08 | +0.393 | 1.035 |
| ATOM | 11 | C9 | UDP | 2 | −1.392 | −13.155 | −3.984 | −0.68 | −0.02 | −0.023 | 1.035 |
| ATOM | 12 | O18 | UDP | 2 | −1.361 | −14.322 | −3.591 | −0.20 | −0.02 | −0.026 | 1.035 |
| ATOM | 13 | C7 | UDP | 2 | −1.399 | −11.973 | −3.181 | −0.52 | +0.00 | +0.000 | 1.035 |
| ATOM | 14 | C5 | UDP | 2 | −1.430 | −10.770 | −3.773 | −0.30 | −0.03 | −0.050 | 1.035 |
| ATOM | 15 | O21 | UDP | 2 | −3.715 | −8.461 | −5.801 | −0.13 | −0.67 | −0.537 | 1.035 |
| ATOM | 16 | H21 | UDP | 2 | −4.434 | −8.724 | −5.112 | +0.07 | +0.52 | +0.424 | 1.035 |
| ATOM | 17 | O23 | UDP | 2 | −2.235 | −6.660 | −6.870 | −0.18 | −0.16 | −0.537 | 1.035 |
| ATOM | 18 | H23 | UDP | 2 | −2.350 | −5.645 | −6.986 | −0.08 | −0.29 | +0.424 | 1.035 |
| ATOM | 19 | C25 | UDP | 2 | 0.321 | −6.586 | −4.231 | −0.29 | +0.04 | +0.113 | 1.035 |
| ATOM | 20 | O28 | UDP | 2 | 1.110 | −5.496 | −4.671 | +0.10 | −0.16 | −0.368 | 1.035 |
| ATOM | 21 | P39 | UDP | 2 | 1.163 | −3.968 | −4.280 | −0.27 | +1.09 | +1.210 | 1.035 |
| ATOM | 22 | O30 | UDP | 2 | −0.079 | −3.653 | −3.517 | −0.24 | −1.09 | −0.850 | 1.035 |
| ATOM | 23 | O31 | UDP | 2 | 2.481 | −3.687 | −3.633 | +0.09 | −0.70 | −0.850 | 1.035 |
| ATOM | 24 | O32 | UDP | 2 | 1.034 | −3.423 | −5.772 | −0.14 | −0.42 | −0.510 | 1.035 |
| ATOM | 25 | P33 | UDP | 2 | 1.363 | −2.014 | −6.452 | −0.60 | +1.55 | +1.210 | 1.035 |
| ATOM | 26 | O36 | UDP | 2 | 0.657 | −1.084 | −5.532 | −0.75 | −2.24 | −0.850 | 1.035 |
| ATOM | 27 | O35 | UDP | 2 | 1.104 | −1.962 | −7.914 | −0.27 | −0.24 | −0.255 | 1.035 |
| ATOM | 28 | O34 | UDP | 2 | 2.968 | −1.863 | −6.332 | −0.12 | −1.09 | −0.850 | 1.035 |
| TER | | | | | | | | | | | |

ENDMDL

| | |
|---|---|
| MODEL | 20 |

| USER | Run = 20 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.085 A | | | | | |
| USER | | | | | | | | | | | |
| USER | Estimated Free Energy of Binding | | | | | = −9.32 kcal/mol [= (1) + (3)] | | | | | |
| USER | Estimated Inhibition Constant, Ki | | | | | = +1.48e−07 [Temperature = 298.15 K] | | | | | |
| USER | | | | | | | | | | | |
| USER | Final Docked Energy | | | | | = −12.24 kcal/mol [= (1) + (2)] | | | | | |
| USER | | | | | | | | | | | |
| USER | (1) Final Intermolecular Energy | | | | | = −11.81 kcal/mol | | | | | |
| USER | (2) Final Internal Energy of Ligand | | | | | = −0.43 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.483852 −7.959618 −5.073061 | | | | | | | | | | |
| USER | NEWDPF quat0 0.714111 −0.332206 0.616186 9.578765 | | | | | | | | | | |
| USER | NEWDPF ndihe 8 | | | | | | | | | | |
| USER | NEWDPF dihe0 5.02 92.51 1.42 −17.32 2.05 −143.68 −56.84 143.32 | | | | | | | | | | |
| USER | | | | | | | | | | | |
| USER | | | | Rank | x | y | z | vdW | Elec | q | RMS |
| ATOM | 1 | C19 | UDP | 2 | −1.387 | −9.253 | −5.896 | −0.45 | +0.10 | +0.174 | 1.085 |
| ATOM | 2 | C1 | UDP | 2 | −2.496 | −8.357 | −5.344 | −0.37 | +0.08 | +0.113 | 1.085 |
| ATOM | 3 | C3 | UDP | 2 | −1.907 | −6.960 | −5.650 | −0.38 | +0.04 | +0.113 | 1.085 |
| ATOM | 4 | O16 | UDP | 2 | −0.206 | −8.601 | −5.445 | +0.02 | −0.11 | −0.227 | 1.085 |
| ATOM | 5 | C14 | UDP | 2 | −0.400 | −7.162 | −5.505 | −0.34 | +0.03 | +0.113 | 1.085 |
| ATOM | 6 | N10 | UDP | 2 | −1.355 | −10.606 | −5.326 | −0.32 | +0.03 | +0.039 | 1.085 |
| ATOM | 7 | C11 | UDP | 2 | −1.293 | −11.683 | −6.166 | −0.66 | −0.06 | −0.073 | 1.085 |
| ATOM | 8 | O17 | UDP | 2 | −1.263 | −11.551 | −7.392 | −0.28 | −0.02 | −0.026 | 1.085 |
| ATOM | 9 | N12 | UDP | 2 | −1.251 | −12.920 | −5.572 | −0.37 | −0.20 | −0.292 | 1.085 |
| ATOM | 10 | H12 | UDP | 2 | −1.186 | −13.783 | −6.234 | −0.06 | +0.09 | +0.393 | 1.085 |
| ATOM | 11 | C9 | UDP | 2 | −1.286 | −13.176 | −4.201 | −0.71 | −0.02 | −0.023 | 1.085 |
| ATOM | 12 | O18 | UDP | 2 | −1.233 | −14.349 | −3.829 | −0.26 | −0.02 | −0.026 | 1.085 |
| ATOM | 13 | C7 | UDP | 2 | −1.357 | −12.011 | −3.377 | −0.54 | +0.00 | +0.000 | 1.085 |
| ATOM | 14 | C5 | UDP | 2 | −1.403 | −10.798 | −3.948 | −0.32 | −0.03 | −0.050 | 1.085 |
| ATOM | 15 | O21 | UDP | 2 | −3.689 | −8.543 | −6.068 | −0.18 | −0.65 | −0.537 | 1.085 |
| ATOM | 16 | H21 | UDP | 2 | −4.249 | −9.285 | −5.624 | +0.07 | +0.46 | +0.424 | 1.085 |
| ATOM | 17 | O23 | UDP | 2 | −2.223 | −6.663 | −7.014 | −0.17 | −0.15 | −0.537 | 1.085 |
| ATOM | 18 | H23 | UDP | 2 | −2.343 | −5.647 | −7.125 | −0.10 | −0.30 | +0.424 | 1.085 |
| ATOM | 19 | C25 | UDP | 2 | 0.168 | −6.541 | −4.228 | −0.28 | +0.04 | +0.113 | 1.085 |
| ATOM | 20 | O28 | UDP | 2 | 1.043 | −5.495 | −4.608 | +0.10 | −0.16 | −0.368 | 1.085 |
| ATOM | 21 | P39 | UDP | 2 | 1.049 | −3.938 | −4.348 | −0.26 | +1.13 | +1.210 | 1.085 |
| ATOM | 22 | O30 | UDP | 2 | −0.247 | −3.581 | −3.703 | −0.24 | −1.31 | −0.850 | 1.085 |
| ATOM | 23 | O31 | UDP | 2 | 2.315 | −3.579 | −3.638 | +0.09 | −0.72 | −0.850 | 1.085 |
| ATOM | 24 | O32 | UDP | 2 | 1.019 | −3.526 | −5.887 | −0.08 | −0.36 | −0.510 | 1.085 |
| ATOM | 25 | P33 | UDP | 2 | 1.407 | −2.185 | −6.666 | −0.63 | +1.36 | +1.210 | 1.085 |
| ATOM | 26 | O36 | UDP | 2 | 0.657 | −1.172 | −5.877 | −0.72 | −2.00 | −0.850 | 1.085 |
| ATOM | 27 | O35 | UDP | 2 | 1.239 | −2.259 | −8.141 | −0.23 | −0.21 | −0.255 | 1.085 |
| ATOM | 28 | O34 | UDP | 2 | 3.003 | −2.035 | −6.460 | −0.10 | −1.08 | −0.850 | 1.085 |
| TER | | | | | | | | | | | |

ENDMDL

| | |
|---|---|
| MODEL | 43 |

| USER | Run = 43 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.024 A | | | | | |
| USER | | | | | | | | | | | |
| USER | Estimated Free Energy of Binding | | | | | = −9.73 kcal/mol [= (1) + (3)] | | | | | |
| USER | Estimated Inhibition Constant, Ki | | | | | = +7.40e−08 [Temperature = 298.15 K] | | | | | |
| USER | | | | | | | | | | | |
| USER | Final Docked Energy | | | | | = −12.18 kcal/mol [= (1) + (2)] | | | | | |
| USER | | | | | | | | | | | |
| USER | (1) Final Intermolecular Energy | | | | | = −12.22 kcal/mol | | | | | |
| USER | (2) Final Internal Energy of Ligand | | | | | = +0.04 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.357224 −7.944272 −5.130388 | | | | | | | | | | |
| USER | NEWDPF quat0 0.594424 0.793523 0.130314 7.278716 | | | | | | | | | | |
| USER | NEWDPF ndihe 8 | | | | | | | | | | |
| USER | NEWDPF dihe0 31.36 88.05 −36.88 −136.01 15.68 −46.12 −0.18 100.92 | | | | | | | | | | |
| USER | | | | | | | | | | | |
| USER | | | | Rank | x | y | z | vdW | Elec | q | RMS |
| ATOM | 1 | C19 | UDP | 2 | −1.489 | −9.275 | −5.885 | −0.45 | +0.10 | +0.174 | 1.024 |
| ATOM | 2 | C1 | UDP | 2 | −2.426 | −8.269 | −5.217 | −0.36 | +0.07 | +0.113 | 1.024 |
| ATOM | 3 | C3 | UDP | 2 | −1.793 | −6.939 | −5.689 | −0.36 | +0.04 | +0.113 | 1.024 |
| ATOM | 4 | O16 | UDP | 2 | −0.206 | −8.704 | −5.661 | +0.01 | −0.11 | −0.227 | 1.024 |
| ATOM | 5 | C14 | UDP | 2 | −0.301 | −7.257 | −5.770 | −0.37 | +0.03 | +0.113 | 1.024 |
| ATOM | 6 | N10 | UDP | 2 | −1.466 | −10.601 | −5.253 | −0.31 | +0.03 | +0.039 | 1.024 |
| ATOM | 7 | C11 | UDP | 2 | −1.336 | −11.713 | −6.038 | −0.66 | −0.06 | −0.073 | 1.024 |
| ATOM | 8 | O17 | UDP | 2 | −1.236 | −11.635 | −7.265 | −0.27 | −0.02 | −0.026 | 1.024 |
| ATOM | 9 | N12 | UDP | 2 | −1.307 | −12.920 | −5.387 | −0.43 | −0.21 | −0.292 | 1.024 |
| ATOM | 10 | H12 | UDP | 2 | −1.188 | −13.811 | −6.004 | −0.23 | +0.15 | +0.393 | 1.024 |
| ATOM | 11 | C9 | UDP | 2 | −1.417 | −13.116 | −4.010 | −0.68 | −0.02 | −0.023 | 1.024 |
| ATOM | 12 | O18 | UDP | 2 | −1.365 | −14.270 | −3.582 | −0.19 | −0.02 | −0.026 | 1.024 |
| ATOM | 13 | C7 | UDP | 2 | −1.557 | −11.917 | −3.246 | −0.51 | +0.00 | +0.000 | 1.024 |
| ATOM | 14 | C5 | UDP | 2 | −1.592 | −10.732 | −3.874 | −0.40 | −0.03 | −0.050 | 1.024 |
| ATOM | 15 | O21 | UDP | 2 | −3.730 | −8.388 | −5.735 | −0.09 | −0.69 | −0.537 | 1.024 |
| ATOM | 16 | H21 | UDP | 2 | −4.374 | −8.675 | −4.984 | +0.07 | +0.50 | +0.424 | 1.024 |
| ATOM | 17 | O23 | UDP | 2 | −2.300 | −6.676 | −7.001 | −0.23 | −0.18 | −0.537 | 1.024 |
| ATOM | 18 | H23 | UDP | 2 | −2.431 | −5.663 | −7.122 | −0.10 | −0.24 | +0.424 | 1.024 |
| ATOM | 19 | C25 | UDP | 2 | 0.508 | −6.630 | −4.633 | −0.31 | +0.04 | +0.113 | 1.024 |
| ATOM | 20 | O28 | UDP | 2 | 0.307 | −5.230 | −4.680 | +0.08 | −0.12 | −0.368 | 1.024 |
| ATOM | 21 | P39 | UDP | 2 | 1.240 | −3.990 | −4.394 | −0.27 | +1.06 | +1.210 | 1.024 |
| ATOM | 22 | O30 | UDP | 2 | 0.543 | −3.117 | −3.407 | −0.23 | −1.31 | −0.850 | 1.024 |
| ATOM | 23 | O31 | UDP | 2 | 2.608 | −4.490 | −4.055 | +0.10 | −0.67 | −0.850 | 1.024 |
| ATOM | 24 | O32 | UDP | 2 | 1.114 | −3.362 | −5.854 | −0.12 | −0.42 | −0.510 | 1.024 |
| ATOM | 25 | P33 | UDP | 2 | 1.406 | −1.907 | −6.446 | −0.62 | +1.62 | +1.210 | 1.024 |
| ATOM | 26 | O36 | UDP | 2 | 0.657 | −1.055 | −5.485 | −0.75 | −2.26 | −0.850 | 1.024 |
| ATOM | 27 | O35 | UDP | 2 | 1.165 | −1.777 | −7.907 | −0.28 | −0.27 | −0.255 | 1.024 |
| ATOM | 28 | O34 | UDP | 2 | 3.003 | −1.711 | −6.293 | −0.13 | −1.11 | −0.850 | 1.024 |
| TER | | | | | | | | | | | |

ENDMDL

| | |
|---|---|
| MODEL | 23 |

| USER | Run = 23 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.035 A | | | | | |
| USER | | | | | | | | | | | |
| USER | Estimated Free Energy of Binding | | | | | = −9.12 kcal/mol [= (1) + (3)] | | | | | |
| USER | Estimated Inhibition Constant, Ki | | | | | = +2.08e−07 [Temperature = 298.15 K] | | | | | |
| USER | | | | | | | | | | | |
| USER | Final Docked Energy | | | | | = −12.09 kcal/mol [= (1) + (2)] | | | | | |
| USER | | | | | | | | | | | |
| USER | (1) Final Intermolecular Energy | | | | | = −11.61 kcal/mol | | | | | |
| USER | (2) Final Internal Energy of Ligand | | | | | = −0.49 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.621461 −7.942733 −4.964907 | | | | | | | | | | |
| USER | NEWDPF quat0 0.490410 −0.625619 0.606712 11.744333 | | | | | | | | | | |
| USER | NEWDPF ndihe 8 | | | | | | | | | | |
| USER | NEWDPF dihe0 13.55 80.03 −5.28 −28.87 −12.31 −134.73 −57.12 −156.35 | | | | | | | | | | |
| USER | | | | | | | | | | | |
| USER | | | | Rank | x | y | z | vdW | Elec | q | RMS |
| ATOM | 1 | C19 | UDP | 2 | −1.443 | −9.246 | −5.757 | −0.44 | +0.10 | +0.174 | 1.035 |
| ATOM | 2 | C1 | UDP | 2 | −2.604 | −8.362 | −5.304 | −0.36 | +0.08 | +0.113 | 1.035 |
| ATOM | 3 | C3 | UDP | 2 | −2.017 | −6.959 | −5.588 | −0.37 | +0.05 | +0.113 | 1.035 |
| ATOM | 4 | O16 | UDP | 2 | −0.309 | −8.568 | −5.230 | +0.04 | −0.11 | −0.227 | 1.035 |
| ATOM | 5 | C14 | UDP | 2 | −0.521 | −7.133 | −5.328 | −0.33 | +0.03 | +0.113 | 1.035 |
| ATOM | 6 | N10 | UDP | 2 | −1.432 | −10.590 | −5.165 | −0.31 | +0.03 | +0.039 | 1.035 |
| ATOM | 7 | C11 | UDP | 2 | −1.589 | −11.679 | −5.977 | −0.63 | −0.06 | −0.073 | 1.035 |
| ATOM | 8 | O17 | UDP | 2 | −1.738 | −11.566 | −7.195 | −0.23 | −0.02 | −0.026 | 1.035 |
| ATOM | 9 | N12 | UDP | 2 | −1.555 | −12.907 | −5.365 | −0.38 | −0.20 | −0.292 | 1.035 |
| ATOM | 10 | H12 | UDP | 2 | −1.663 | −13.781 | −6.007 | −0.24 | +0.01 | +0.393 | 1.035 |
| ATOM | 11 | C9 | UDP | 2 | −1.396 | −13.142 | −3.999 | −0.68 | −0.02 | −0.023 | 1.035 |
| ATOM | 12 | O18 | UDP | 2 | −1.381 | −14.311 | −3.609 | −0.20 | −0.02 | −0.026 | 1.035 |
| ATOM | 13 | C7 | UDP | 2 | −1.244 | −11.965 | −3.204 | −0.52 | +0.00 | +0.000 | 1.035 |
| ATOM | 14 | C5 | UDP | 2 | −1.281 | −10.760 | −3.792 | −0.21 | −0.03 | −0.050 | 1.035 |
| ATOM | 15 | O21 | UDP | 2 | −3.737 | −8.578 | −6.112 | −0.18 | −0.65 | −0.537 | 1.035 |
| ATOM | 16 | H21 | UDP | 2 | −4.386 | −9.217 | −5.630 | +0.07 | +0.49 | +0.424 | 1.035 |
| ATOM | 17 | O23 | UDP | 2 | −2.234 | −6.688 | −6.976 | −0.20 | −0.17 | −0.537 | 1.035 |
| ATOM | 18 | H23 | UDP | 2 | −2.566 | −5.721 | −7.089 | −0.10 | −0.13 | +0.424 | 1.035 |
| ATOM | 19 | C25 | UDP | 2 | −0.060 | −6.484 | −4.022 | −0.26 | +0.04 | +0.113 | 1.035 |
| ATOM | 20 | O28 | UDP | 2 | 0.829 | −5.433 | −4.352 | +0.10 | −0.16 | −0.368 | 1.035 |
| ATOM | 21 | P39 | UDP | 2 | 0.889 | −3.903 | −3.970 | −0.23 | +1.18 | +1.210 | 1.035 |
| ATOM | 22 | O30 | UDP | 2 | −0.469 | −3.498 | −3.507 | −0.24 | −1.30 | −0.850 | 1.035 |
| ATOM | 23 | O31 | UDP | 2 | 2.045 | −3.688 | −3.046 | +0.11 | −0.64 | −0.850 | 1.035 |
| ATOM | 24 | O32 | UDP | 2 | 1.131 | −3.382 | −5.457 | −0.19 | −0.52 | −0.510 | 1.035 |
| ATOM | 25 | P33 | UDP | 2 | 1.593 | −1.976 | −6.060 | −0.61 | +1.70 | +1.210 | 1.035 |
| ATOM | 26 | O36 | UDP | 2 | 0.657 | −1.051 | −5.368 | −0.75 | −2.32 | −0.850 | 1.035 |
| ATOM | 27 | O35 | UDP | 2 | 1.711 | −1.951 | −7.541 | −0.17 | −0.29 | −0.255 | 1.035 |
| ATOM | 28 | O34 | UDP | 2 | 3.111 | −1.782 | −5.541 | −0.12 | −1.03 | −0.850 | 1.035 |
| TER | | | | | | | | | | | |

ENDMDL

| | |
|---|---|
| MODEL | 49 |

| USER | Run = 49 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.011 A | | | | | |
| USER | | | | | | | | | | | |
| USER | Estimated Free Energy of Binding | | | | | = −9.03 kcal/mol [= (1) + (3)] | | | | | |
| USER | Estimated Inhibition Constant, Ki | | | | | = +2.42e−07 [Temperature = 298.15 K] | | | | | |
| USER | | | | | | | | | | | |
| USER | Final Docked Energy | | | | | = −12.05 kcal/mol [= (1) + (2)] | | | | | |
| USER | | | | | | | | | | | |
| USER | (1) Final Intermolecular Energy | | | | | = −11.52 kcal/mol | | | | | |
| USER | (2) Final Internal Energy of Ligand | | | | | = −0.53 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.133363 −8.081972 −5.142666 | | | | | | | | | | |
| USER | NEWDPF quat0 0.357796 −0.710427 −0.606032 0.862853 | | | | | | | | | | |
| USER | NEWDPF ndihe 8 | | | | | | | | | | |
| USER | NEWDPF dihe0 23.79 88.05 9.77 −34.73 −1.43 −134.59 −34.20 −74.53 | | | | | | | | | | |
| USER | | | | | | | | | | | |
| USER | | | | Rank | x | y | z | vdW | Elec | q | RMS |
| ATOM | 1 | C19 | UDP | 2 | −1.222 | −9.461 | −5.814 | −0.46 | +0.10 | +0.174 | 1.011 |
| ATOM | 2 | C1 | UDP | 2 | −2.195 | −8.388 | −5.326 | −0.36 | +0.07 | +0.113 | 1.011 |
| ATOM | 3 | C3 | UDP | 2 | −1.474 | −7.110 | −5.814 | −0.38 | +0.03 | +0.113 | 1.011 |
| ATOM | 4 | O16 | UDP | 2 | 0.046 | −8.903 | −5.487 | +0.02 | −0.12 | −0.227 | 1.011 |
| ATOM | 5 | C14 | UDP | 2 | 0.007 | −7.466 | −5.706 | −0.39 | +0.04 | +0.113 | 1.011 |
| ATOM | 6 | N10 | UDP | 2 | −1.308 | −10.738 | −5.094 | −0.32 | +0.03 | +0.039 | 1.011 |
| ATOM | 7 | C11 | UDP | 2 | −1.481 | −11.894 | −5.803 | −0.64 | −0.06 | −0.073 | 1.011 |
| ATOM | 8 | O17 | UDP | 2 | −1.565 | −11.896 | −7.033 | −0.25 | −0.02 | −0.026 | 1.011 |
| ATOM | 9 | N12 | UDP | 2 | −1.540 | −13.055 | −5.073 | −0.44 | −0.20 | −0.292 | 1.011 |
| ATOM | 10 | H12 | UDP | 2 | −1.661 | −13.982 | −5.632 | −0.27 | +0.12 | +0.393 | 1.011 |
| ATOM | 11 | C9 | UDP | 2 | −1.460 | −13.159 | −3.684 | −0.65 | −0.02 | −0.023 | 1.011 |
| ATOM | 12 | O18 | UDP | 2 | −1.524 | −14.283 | −3.182 | −0.11 | −0.02 | −0.026 | 1.011 |
| ATOM | 13 | C7 | UDP | 2 | −1.287 | −11.916 | −3.001 | −0.51 | +0.00 | +0.000 | 1.011 |
| ATOM | 14 | C5 | UDP | 2 | −1.234 | −10.776 | −3.705 | −0.16 | −0.03 | −0.050 | 1.011 |
| ATOM | 15 | O21 | UDP | 2 | −3.438 | −8.514 | −5.977 | −0.23 | −0.60 | −0.537 | 1.011 |
| ATOM | 16 | H21 | UDP | 2 | −4.143 | −8.856 | −5.308 | +0.07 | −0.48 | +0.424 | 1.011 |
| ATOM | 17 | O23 | UDP | 2 | −1.826 | −6.930 | −7.190 | −0.09 | −0.11 | −0.537 | 1.011 |
| ATOM | 18 | H23 | UDP | 2 | −1.912 | −5.926 | −7.394 | −0.19 | −0.29 | +0.424 | 1.011 |
| ATOM | 19 | C25 | UDP | 2 | 0.705 | −6.778 | −4.532 | −0.33 | +0.04 | +0.113 | 1.011 |
| ATOM | 20 | O28 | UDP | 2 | 1.254 | −5.563 | −5.007 | +0.09 | −0.16 | −0.368 | 1.011 |
| ATOM | 21 | P39 | UDP | 2 | 1.149 | −4.063 | −4.526 | −0.27 | +1.02 | +1.210 | 1.011 |
| ATOM | 22 | O30 | UDP | 2 | −0.136 | −3.915 | −3.785 | −0.22 | −0.95 | −0.850 | 1.011 |
| ATOM | 23 | O31 | UDP | 2 | 2.418 | −3.698 | −3.824 | +0.09 | −0.72 | −0.850 | 1.011 |
| ATOM | 24 | O32 | UDP | 2 | 1.005 | −3.443 | −5.987 | −0.08 | −0.35 | −0.510 | 1.011 |
| ATOM | 25 | P33 | UDP | 2 | 1.401 | −2.028 | −6.617 | −0.62 | +1.48 | +1.210 | 1.011 |
| ATOM | 26 | O36 | UDP | 2 | 0.787 | −1.099 | −5.632 | −0.77 | −2.11 | −0.850 | 1.011 |
| ATOM | 27 | O35 | UDP | 2 | 1.096 | −1.892 | −8.065 | −0.29 | −0.24 | −0.255 | 1.011 |
| ATOM | 28 | O34 | UDP | 2 | 3.015 | −1.982 | −6.549 | −0.08 | −1.10 | −0.850 | 1.011 |
| TER | | | | | | | | | | | |

ENDMDL

| | |
|---|---|
| MODEL | 40 |

| USER | Run = 40 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.076 A | | | | | |
| USER | | | | | | | | | | | |
| USER | Estimated Free Energy of Binding | | | | | = −9.15 kcal/mol [= (1) + (3)] | | | | | |
| USER | Estimated Inhibition Constant, Ki | | | | | = +1.97e−07 [Temperature = 298.15 K] | | | | | |
| USER | | | | | | | | | | | |
| USER | Final Docked Energy | | | | | = −12.01 kcal/mol [= (1) + (2)] | | | | | |
| USER | | | | | | | | | | | |
| USER | (1) Final Intermolecular Energy | | | | | = −11.64 kcal/mol | | | | | |
| USER | (2) Final Internal Energy of Ligand | | | | | = −0.37 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.312436 −7.878495 −5.296677 | | | | | | | | | | |
| USER | NEWDPF quat0 −0.187984 −0.871430 0.453070 4.743227 | | | | | | | | | | |
| USER | NEWDPF ndihe 8 | | | | | | | | | | |
| USER | NEWDPF dihe0 32.74 63.17 −23.72 −27.42 4.98 −121.16 −49.13 98.21 | | | | | | | | | | |
| USER | | | | | | | | | | | |
| USER | | | | Rank | x | y | z | vdW | Elec | q | RMS |
| ATOM | 1 | C19 | UDP | 2 | −1.296 | −9.273 | −5.940 | −0.46 | +0.10 | +0.174 | 1.076 |
| ATOM | 2 | C1 | UDP | 2 | −2.346 | −8.238 | −5.537 | −0.38 | +0.07 | +0.113 | 1.076 |
| ATOM | 3 | C3 | UDP | 2 | −1.655 | −6.937 | −6.010 | −0.32 | +0.03 | +0.113 | 1.076 |
| ATOM | 4 | O16 | UDP | 2 | −0.078 | −8.650 | −5.550 | +0.01 | −0.11 | −0.227 | 1.076 |
| ATOM | 5 | C14 | UDP | 2 | −0.168 | −7.221 | −5.804 | −0.37 | +0.03 | +0.113 | 1.076 |
| ATOM | 6 | N10 | UDP | 2 | −1.369 | −10.538 | −5.198 | −0.31 | +0.03 | +0.039 | 1.076 |
| ATOM | 7 | C11 | UDP | 2 | −1.311 | −11.719 | −5.884 | −0.66 | −0.06 | −0.073 | 1.076 |
| ATOM | 8 | O17 | UDP | 2 | −1.198 | −11.754 | −7.111 | −0.24 | −0.02 | −0.026 | 1.076 |
| ATOM | 9 | N12 | UDP | 2 | −1.369 | −12.866 | −5.131 | −0.47 | −0.21 | −0.292 | 1.076 |
| ATOM | 10 | H12 | UDP | 2 | −1.307 | −13.812 | −5.669 | −0.25 | +0.19 | +0.393 | 1.076 |
| ATOM | 11 | C9 | UDP | 2 | −1.502 | −12.935 | −3.745 | −0.63 | −0.02 | −0.023 | 1.076 |
| ATOM | 12 | O18 | UDP | 2 | −1.532 | −14.049 | −3.219 | −0.12 | −0.02 | −0.026 | 1.076 |
| ATOM | 13 | C7 | UDP | 2 | −1.563 | −11.668 | −3.087 | −0.47 | +0.00 | +0.000 | 1.076 |
| ATOM | 14 | C5 | UDP | 2 | −1.512 | −10.541 | −3.814 | −0.33 | −0.03 | −0.050 | 1.076 |
| ATOM | 15 | O21 | UDP | 2 | −3.539 | −8.435 | −6.259 | −0.18 | −0.66 | −0.537 | 1.076 |
| ATOM | 16 | H21 | UDP | 2 | −4.305 | −8.651 | −5.606 | +0.07 | +0.56 | +0.424 | 1.076 |
| ATOM | 17 | O23 | UDP | 2 | −1.929 | −6.802 | −7.408 | −0.09 | −0.09 | −0.537 | 1.076 |
| ATOM | 18 | H23 | UDP | 2 | −2.443 | −5.927 | −7.574 | −0.15 | −0.05 | +0.424 | 1.076 |
| ATOM | 19 | C25 | UDP | 2 | 0.423 | −6.478 | −4.605 | −0.30 | +0.04 | +0.113 | 1.076 |
| ATOM | 20 | O28 | UDP | 2 | 1.240 | −5.434 | −5.104 | +0.09 | −0.15 | −0.368 | 1.076 |
| ATOM | 21 | P39 | UDP | 2 | 1.495 | −3.944 | −4.649 | −0.30 | +1.03 | +1.210 | 1.076 |
| ATOM | 22 | O30 | UDP | 2 | 0.369 | −3.544 | −3.757 | −0.24 | −1.23 | −0.850 | 1.076 |
| ATOM | 23 | O31 | UDP | 2 | 2.889 | −3.840 | −4.120 | +0.07 | −0.75 | −0.850 | 1.076 |
| ATOM | 24 | O32 | UDP | 2 | 1.291 | −3.306 | −6.095 | −0.06 | −0.39 | −0.510 | 1.076 |
| ATOM | 25 | P33 | UDP | 2 | 1.256 | −1.799 | −6.629 | −0.59 | +1.62 | +1.210 | 1.076 |
| ATOM | 26 | O36 | UDP | 2 | 0.282 | −1.189 | −5.687 | −0.43 | −2.37 | −0.850 | 1.076 |
| ATOM | 27 | O35 | UDP | 2 | 1.060 | −1.671 | −8.096 | −0.34 | −0.27 | −0.255 | 1.076 |
| ATOM | 28 | O34 | UDP | 2 | 2.752 | −1.239 | −6.382 | −0.18 | −1.24 | −0.850 | 1.076 |
| TER | | | | | | | | | | | |

ENDMDL

| | |
|---|---|
| MODEL | 25 |

| USER | Run = 25 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.069 A | | | | | |
| USER | | | | | | | | | | | |
| USER | Estimated Free Energy of Binding | | | | | = −9.16 kcal/mol [= (1) + (3)] | | | | | |
| USER | Estimated Inhibition Constant, Ki | | | | | = +1.92e−07 [Temperature = 298.15 K] | | | | | |
| USER | | | | | | | | | | | |
| USER | Final Docked Energy | | | | | = −11.88 kcal/mol [= (1) + (2)] | | | | | |
| USER | | | | | | | | | | | |
| USER | (1) Final Intermolecular Energy | | | | | = −11.65 kcal/mol | | | | | |
| USER | (2) Final Internal Energy of Ligand | | | | | = −0.23 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.156154 −8.058689 −5.522049 | | | | | | | | | | |
| USER | NEWDPF quat0 0.486652 −0.576502 0.656365 −0.562859 | | | | | | | | | | |
| USER | NEWDPF ndihe 8 | | | | | | | | | | |
| USER | NEWDPF dihe0 15.11 110.98 9.80 −54.97 11.25 −118.01 −24.54 −155.02 | | | | | | | | | | |
| USER | | | | | | | | | | | |
| USER | | | | Rank | x | y | z | vdW | Elec | q | RMS |
| ATOM | 1 | C19 | UDP | 2 | −1.252 | −9.444 | −6.178 | −0.49 | +0.10 | +0.174 | 1.069 |
| ATOM | 2 | C1 | UDP | 2 | −2.220 | −8.370 | −5.685 | −0.39 | +0.07 | +0.113 | 1.069 |
| ATOM | 3 | C3 | UDP | 2 | −1.511 | −7.095 | −6.198 | −0.39 | +0.03 | +0.113 | 1.069 |
| ATOM | 4 | O16 | UDP | 2 | 0.020 | −8.880 | −5.878 | −0.02 | −0.12 | −0.227 | 1.069 |
| ATOM | 5 | C14 | UDP | 2 | −0.026 | −7.446 | −6.110 | −0.42 | +0.03 | +0.113 | 1.069 |
| ATOM | 6 | N10 | UDP | 2 | −1.323 | −10.714 | −5.443 | −0.34 | +0.03 | +0.039 | 1.069 |
| ATOM | 7 | C11 | UDP | 2 | −1.241 | −11.891 | −6.135 | −0.63 | −0.06 | −0.073 | 1.069 |
| ATOM | 8 | O17 | UDP | 2 | −1.107 | −11.917 | −7.360 | −0.13 | −0.02 | −0.026 | 1.069 |
| ATOM | 9 | N12 | UDP | 2 | −1.299 | −13.042 | −5.390 | −0.41 | −0.20 | −0.292 | 1.069 |
| ATOM | 10 | H12 | UDP | 2 | −1.218 | −13.984 | −5.932 | −0.18 | +0.13 | +0.393 | 1.069 |
| ATOM | 11 | C9 | UDP | 2 | −1.454 | −13.121 | −4.006 | −0.67 | −0.02 | −0.023 | 1.069 |
| ATOM | 12 | O18 | UDP | 2 | −1.481 | −14.238 | −3.487 | −0.16 | −0.02 | −0.026 | 1.069 |
| ATOM | 13 | C7 | UDP | 2 | −1.541 | −11.858 | −3.342 | −0.51 | +0.00 | +0.000 | 1.069 |
| ATOM | 14 | C5 | UDP | 2 | −1.490 | −10.727 | −4.062 | −0.37 | −0.03 | −0.050 | 1.069 |
| ATOM | 15 | O21 | UDP | 2 | −3.473 | −8.506 | −6.314 | −0.24 | −0.62 | −0.537 | 1.069 |
| ATOM | 16 | H21 | UDP | 2 | −4.129 | −8.977 | −5.675 | +0.07 | +0.48 | +0.424 | 1.069 |
| ATOM | 17 | O23 | UDP | 2 | −1.885 | −6.930 | −7.569 | −0.12 | −0.12 | −0.537 | 1.069 |
| ATOM | 18 | H23 | UDP | 2 | −1.613 | −5.991 | −7.889 | −0.25 | −0.24 | +0.424 | 1.069 |
| ATOM | 19 | C25 | UDP | 2 | 0.689 | −6.744 | −4.955 | −0.34 | +0.04 | +0.113 | 1.069 |
| ATOM | 20 | O28 | UDP | 2 | 1.030 | −5.439 | −5.386 | +0.08 | −0.12 | −0.368 | 1.069 |
| ATOM | 21 | P39 | UDP | 2 | 1.085 | −4.038 | −4.663 | −0.27 | +1.01 | +1.210 | 1.069 |
| ATOM | 22 | O30 | UDP | 2 | −0.165 | −3.890 | −3.865 | −0.23 | −0.99 | −0.850 | 1.069 |
| ATOM | 23 | O31 | UDP | 2 | 2.396 | −3.919 | −3.953 | +0.10 | −0.70 | −0.850 | 1.069 |
| ATOM | 24 | O32 | UDP | 2 | 0.981 | −3.172 | −5.997 | −0.16 | −0.43 | −0.510 | 1.069 |
| ATOM | 25 | P33 | UDP | 2 | 1.508 | −1.718 | −6.400 | −0.53 | +1.76 | +1.210 | 1.069 |
| ATOM | 26 | O36 | UDP | 2 | 1.009 | −0.910 | −5.257 | −0.73 | −2.06 | −0.850 | 1.069 |
| ATOM | 27 | O35 | UDP | 2 | 1.195 | −1.314 | −7.796 | −0.29 | −0.34 | −0.255 | 1.069 |
| ATOM | 28 | O34 | UDP | 2 | 3.119 | −1.843 | −6.380 | −0.12 | −1.11 | −0.850 | 1.069 |
| TER | | | | | | | | | | | |

ENDMDL

| | |
|---|---|
| MODEL | 46 |

| USER | Run = 46 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 0.952 A | | | | | |
| USER | | | | | | | | | | | |
| USER | Estimated Free Energy of Binding | | | | | = −9.50 kcal/mol [= (1) + (3)] | | | | | |
| USER | Estimated Inhibition Constant, Ki | | | | | = +1.08e−07 [Temperature = 298.15 K] | | | | | |
| USER | | | | | | | | | | | |
| USER | Final Docked Energy | | | | | = −11.86 kcal/mol [= (1) + (2)] | | | | | |
| USER | | | | | | | | | | | |
| USER | (1) Final Intermolecular Energy | | | | | = −12.00 kcal/mol | | | | | |
| USER | (2) Final Internal Energy of Ligand | | | | | = +0.14 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.261732 −8.091114 −5.113627 | | | | | | | | | | |
| USER | NEWDPF quat0 0.142848 0.894298 −0.424058 2.522636 | | | | | | | | | | |
| USER | NEWDPF ndihe 8 | | | | | | | | | | |
| USER | NEWDPF dihe0 −2.88 93.27 −31.66 −147.50 7.10 −47.49 11.15 −142.38 | | | | | | | | | | |
| USER | | | | | | | | | | | |
| USER | | | | Rank | x | y | z | vdW | Elec | q | RMS |
| ATOM | 1 | C19 | UDP | 2 | −1.397 | −9.468 | −5.781 | −0.45 | +0.10 | +0.174 | 0.952 |
| ATOM | 2 | C1 | UDP | 2 | −2.334 | −8.387 | −5.244 | −0.36 | +0.07 | +0.113 | 0.952 |
| ATOM | 3 | C3 | UDP | 2 | −1.627 | −7.115 | −5.766 | −0.39 | +0.04 | +0.113 | 0.952 |
| ATOM | 4 | O16 | UDP | 2 | −0.109 | −8.923 | −5.518 | +0.01 | −0.12 | −0.227 | 0.952 |
| ATOM | 5 | C14 | UDP | 2 | −0.145 | −7.485 | −5.733 | −0.39 | +0.04 | +0.113 | 0.952 |
| ATOM | 6 | N10 | UDP | 2 | −1.459 | −10.745 | −5.058 | −0.31 | +0.03 | +0.039 | 0.952 |
| ATOM | 7 | C11 | UDP | 2 | −1.501 | −11.913 | −5.769 | −0.63 | −0.06 | −0.073 | 0.952 |
| ATOM | 8 | O17 | UDP | 2 | −1.487 | −11.925 | −7.002 | −0.24 | −0.02 | −0.026 | 0.952 |
| ATOM | 9 | N12 | UDP | 2 | −1.544 | −13.072 | −5.036 | −0.44 | −0.20 | −0.292 | 0.952 |
| ATOM | 10 | H12 | UDP | 2 | −1.561 | −14.008 | −5.594 | −0.27 | +0.14 | +0.393 | 0.952 |
| ATOM | 11 | C9 | UDP | 2 | −1.569 | −13.166 | −3.644 | −0.63 | −0.02 | −0.023 | 0.952 |
| ATOM | 12 | O18 | UDP | 2 | −1.601 | −14.289 | −3.139 | −0.10 | −0.02 | −0.026 | 0.952 |
| ATOM | 13 | C7 | UDP | 2 | −1.530 | −11.912 | −2.960 | −0.50 | +0.00 | +0.000 | 0.952 |
| ATOM | 14 | C5 | UDP | 2 | −1.493 | −10.773 | −3.668 | −0.32 | −0.03 | −0.050 | 0.952 |
| ATOM | 15 | O21 | UDP | 2 | −3.609 | −8.501 | −5.832 | −0.18 | −0.63 | −0.537 | 0.952 |
| ATOM | 16 | H21 | UDP | 2 | −4.156 | −9.216 | −5.331 | +0.08 | +0.45 | +0.424 | 0.952 |
| ATOM | 17 | O23 | UDP | 2 | −2.045 | −6.930 | −7.123 | −0.17 | −0.16 | −0.537 | 0.952 |
| ATOM | 18 | H23 | UDP | 2 | −2.048 | −5.927 | −7.347 | −0.18 | −0.24 | +0.424 | 0.952 |
| ATOM | 19 | C25 | UDP | 2 | 0.617 | −6.805 | −4.595 | −0.33 | +0.04 | +0.113 | 0.952 |
| ATOM | 20 | O28 | UDP | 2 | 0.258 | −5.436 | −4.592 | +0.09 | −0.11 | −0.368 | 0.952 |
| ATOM | 21 | P39 | UDP | 2 | 1.046 | −4.109 | −4.259 | −0.26 | +1.02 | +1.210 | 0.952 |
| ATOM | 22 | O30 | UDP | 2 | 0.138 | −3.230 | −3.468 | −0.18 | −1.48 | −0.850 | 0.952 |
| ATOM | 23 | O31 | UDP | 2 | 2.369 | −4.475 | −3.668 | +0.10 | −0.63 | −0.850 | 0.952 |
| ATOM | 24 | O32 | UDP | 2 | 1.119 | −3.593 | −5.766 | −0.07 | −0.37 | −0.510 | 0.952 |
| ATOM | 25 | P33 | UDP | 2 | 1.509 | −2.188 | −6.421 | −0.62 | +1.45 | +1.210 | 0.952 |
| ATOM | 26 | O36 | UDP | 2 | 0.656 | −1.262 | −5.632 | −0.76 | −2.17 | −0.850 | 0.952 |
| ATOM | 27 | O35 | UDP | 2 | 1.461 | −2.168 | −7.906 | −0.21 | −0.24 | −0.255 | 0.952 |
| ATOM | 28 | O34 | UDP | 2 | 3.075 | −1.992 | −6.076 | −0.11 | −1.06 | −0.850 | 0.952 |
| TER | | | | | | | | | | | |

ENDMDL

| | |
|---|---|
| MODEL | 9 |

| USER | Run = 9 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure | | | | | = 1.083 A | | | | | |
| USER | | | | | | | | | | | |
| USER | Estimated Free Energy of Binding | | | | | = −9.17 kcal/mol [= (1) + (3)] | | | | | |
| USER | Estimated Inhibition Constant, Ki | | | | | = +1.90e−07 (Temperature = 298.15 K] | | | | | |
| USER | | | | | | | | | | | |
| USER | Final Docked Energy | | | | | = −11.52 kcal/mol [= (1) + (2)] | | | | | |
| USER | | | | | | | | | | | |
| USER | (1) Final Intermolecular Energy | | | | | = −11.66 kcal/mol | | | | | |
| USER | (2) Final Internal Energy of Ligand | | | | | = +0.14 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.395621 −7.942467 −4.768348 | | | | | | | | | | |
| USER | NEWDPF quat0 −0.734431 −0.655024 −0.177634 −10.594018 | | | | | | | | | | |
| USER | NEWDPF ndihe8 | | | | | | | | | | |
| USER | NEWDPF dihe0 13.23 100.78 −16.16 174.84 13.07 −25.15 17.22 41.37 | | | | | | | | | | |
| USER | | | | | | | | | | | |
| USER | | | | Rank | x | y | z | vdW | Elec | q | RMS |
| ATOM | 1 | C19 | UDP | 2 | −1.525 | −9.227 | −5.600 | −0.42 | +0.10 | +0.174 | 1.083 |
| ATOM | 2 | C1 | UDP | 2 | −2.461 | −8.277 | −4.854 | −0.33 | +0.07 | +0.113 | 1.083 |
| ATOM | 3 | C3 | UDP | 2 | −1.856 | −6.912 | −5.256 | −0.35 | +0.04 | +0.113 | 1.083 |
| ATOM | 4 | O16 | UDP | 2 | −0.245 | −8.651 | −5.367 | +0.03 | −0.11 | −0.227 | 1.083 |
| ATOM | 5 | C14 | UDP | 2 | −0.362 | −7.202 | −5.386 | −0.34 | +0.03 | +0.113 | 1.083 |
| ATOM | 6 | N10 | UDP | 2 | −1.471 | −10.588 | −5.051 | −0.30 | +0.03 | +0.039 | 1.083 |
| ATOM | 7 | C11 | UDP | 2 | −1.397 | −11.651 | −5.907 | −0.65 | −0.06 | −0.073 | 1.083 |
| ATOM | 8 | O17 | UDP | 2 | −1.373 | −11.501 | −7.131 | −0.26 | −0.02 | −0.026 | 1.083 |
| ATOM | 9 | N12 | UDP | 2 | −1.336 | −12.896 | −5.331 | −0.45 | −0.21 | −0.292 | 1.083 |
| ATOM | 10 | H12 | UDP | 2 | −1.260 | −13.748 | −6.007 | −0.24 | +0.13 | +0.393 | 1.083 |
| ATOM | 11 | C9 | UDP | 2 | −1.362 | −13.173 | −3.965 | −0.69 | −0.02 | −0.023 | 1.083 |
| ATOM | 12 | O18 | UDP | 2 | −1.290 | −14.351 | −3.610 | −0.21 | −0.02 | −0.026 | 1.083 |
| ATOM | 13 | C7 | UDP | 2 | −1.447 | −12.021 | −3.123 | −0.52 | +0.00 | +0.000 | 1.083 |
| ATOM | 14 | C5 | UDP | 2 | −1.513 | −10.801 | −3.676 | −0.34 | −0.03 | −0.050 | 1.083 |
| ATOM | 15 | O21 | UDP | 2 | −3.774 | −8.383 | −5.352 | −0.11 | −0.63 | −0.537 | 1.083 |
| ATOM | 16 | H21 | UDP | 2 | −4.328 | −8.994 | −4.734 | +0.08 | +0.45 | +0.424 | 1.083 |
| ATOM | 17 | O23 | UDP | 2 | −2.394 | −6.577 | −6.540 | −0.18 | −0.23 | −0.537 | 1.083 |
| ATOM | 18 | H23 | UDP | 2 | −2.342 | −5.559 | −6.678 | −0.05 | −0.31 | +0.424 | 1.083 |
| ATOM | 19 | C25 | UDP | 2 | 0.462 | −6.634 | −4.229 | −0.30 | +0.04 | +0.113 | 1.083 |
| ATOM | 20 | O28 | UDP | 2 | −0.050 | −5.350 | −3.924 | +0.10 | −0.14 | −0.368 | 1.083 |
| ATOM | 21 | P39 | UDP | 2 | 0.596 | −3.911 | −3.866 | −0.19 | +1.24 | +1.210 | 1.083 |
| ATOM | 22 | O30 | UDP | 2 | −0.450 | −2.965 | −3.382 | −0.10 | −1.94 | −0.850 | 1.083 |
| ATOM | 23 | O31 | UDP | 2 | 1.881 | −3.995 | −3.105 | +0.12 | −0.61 | −0.850 | 1.083 |
| ATOM | 24 | O32 | UDP | 2 | 0.775 | −3.759 | −5.442 | −0.13 | −0.38 | −0.510 | 1.083 |
| ATOM | 25 | P33 | UDP | 2 | 1.412 | −2.619 | −6.365 | −0.52 | +1.23 | +1.210 | 1.083 |
| ATOM | 26 | O36 | UDP | 2 | 0.726 | −1.411 | −5.835 | −0.75 | −1.91 | −0.850 | 1.083 |
| ATOM | 27 | O35 | UDP | 2 | 1.384 | −2.918 | −7.820 | −0.13 | −0.16 | −0.255 | 1.083 |
| ATOM | 28 | O34 | UDP | 2 | 2.985 | −2.619 | −5.994 | −0.04 | −0.98 | −0.850 | 1.083 |
| TER | | | | | | | | | | | |

ENDMDL

| | |
|---|---|
| MODEL | 31 |

| USER | Run = 31 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure = 1.021 A | | | | | | | | | | |
| USER | | | | | | | | | | | |
| USER | Estimated Free Energy of Binding | | | | | = −8.83 kcal/mol [= (1) + (3)] | | | | | |
| USER | Estimated Inhibition Constant, Ki | | | | | = +3.39e−07 [Temperature = 298.15 K] | | | | | |
| USER | | | | | | | | | | | |
| USER | Final Docked Energy | | | | | = −11.29 kcal/mol [= (1) + (2)] | | | | | |
| USER | | | | | | | | | | | |
| USER | (1) Final Intermolecular Energy | | | | | = −11.32 kcal/mol | | | | | |
| USER | (2) Final Internal Energy of Ligand | | | | | = +0.03 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.208769 −8.028328 −5.321952 | | | | | | | | | | |
| USER | NEWDPF quat0 −0.916013 0.394792 −0.071126 −1.467934 | | | | | | | | | | |
| USER | NEWDPF ndihe8 | | | | | | | | | | |
| USER | NEWDPF dihe0 −7.98 118.37 −99.38 −100.30 11.17 −4.82 −31.41 −116.74 | | | | | | | | | | |
| USER | | | | | | | | | | | |
| USER | | | | Rank | x | y | z | vdW | Elec | q | RMS |
| ATOM | 1 | C19 | UDP | 2 | −1.282 | −9.396 | −6.018 | −0.47 | +0.10 | +0.174 | 1.021 |
| ATOM | 2 | C1 | UDP | 2 | −2.267 | −8.343 | −5.510 | −0.38 | +0.07 | +0.113 | 1.021 |
| ATOM | 3 | C3 | UDP | 2 | −1.561 | −7.048 | −5.976 | −0.38 | +0.03 | +0.113 | 1.021 |
| ATOM | 4 | O16 | UDP | 2 | −0.021 | −8.830 | −5.682 | +0.00 | −0.12 | −0.227 | 1.021 |
| ATOM | 5 | C14 | UDP | 2 | −0.075 | −7.390 | −5.875 | −0.39 | +0.03 | +0.113 | 1.021 |
| ATOM | 6 | N10 | UDP | 2 | −1.354 | −10.686 | −5.321 | −0.33 | +0.03 | +0.039 | 1.021 |
| ATOM | 7 | C11 | UDP | 2 | −1.253 | −11.842 | −6.044 | −0.66 | −0.06 | −0.073 | 1.021 |
| ATOM | 8 | O17 | UDP | 2 | −1.101 | −11.833 | −7.267 | −0.17 | −0.02 | −0.026 | 1.021 |
| ATOM | 9 | N12 | UDP | 2 | −1.313 | −13.014 | −5.333 | −0.43 | −0.20 | −0.292 | 1.021 |
| ATOM | 10 | H12 | UDP | 2 | −1.217 | −13.940 | −5.900 | −0.20 | +0.15 | +0.393 | 1.021 |
| ATOM | 11 | C9 | UDP | 2 | −1.488 | −13.133 | −3.954 | −0.66 | −0.02 | −0.023 | 1.021 |
| ATOM | 12 | O18 | UDP | 2 | −1.514 | −14.265 | −3.467 | −0.16 | −0.02 | −0.026 | 1.021 |
| ATOM | 13 | C7 | UDP | 2 | −1.594 | −11.890 | −3.256 | −0.51 | +0.00 | +0.000 | 1.021 |
| ATOM | 14 | C5 | UDP | 2 | −1.541 | −10.739 | −3.943 | −0.38 | −0.03 | −0.050 | 1.021 |
| ATOM | 15 | O21 | UDP | 2 | −3.509 | −8.471 | −6.163 | −0.21 | −0.64 | −0.537 | 1.021 |
| ATOM | 16 | H21 | UDP | 2 | −4.031 | −9.262 | −5.758 | +0.07 | +0.44 | +0.424 | 1.021 |
| ATOM | 17 | O23 | UDP | 2 | −1.915 | −6.848 | −7.348 | −0.10 | −0.10 | −0.537 | 1.021 |
| ATOM | 18 | H23 | UDP | 2 | −1.537 | −5.948 | −7.672 | −0.23 | −0.33 | +0.424 | 1.021 |
| ATOM | 19 | C25 | UDP | 2 | 0.616 | −6.715 | −4.689 | −0.32 | +0.04 | +0.113 | 1.021 |
| ATOM | 20 | O28 | UDP | 2 | 1.094 | −5.457 | −5.126 | +0.09 | −0.14 | −0.368 | 1.021 |
| ATOM | 21 | P39 | UDP | 2 | 2.523 | −4.919 | −5.527 | −0.36 | +0.88 | +1.210 | 1.021 |
| ATOM | 22 | O30 | UDP | 2 | 3.158 | −4.362 | −4.299 | +0.06 | −0.77 | −0.850 | 1.021 |
| ATOM | 23 | O31 | UDP | 2 | 3.246 | −5.997 | −6.271 | +0.00 | −0.69 | −0.850 | 1.021 |
| ATOM | 24 | O32 | UDP | 2 | 1.988 | −3.732 | −6.446 | +0.01 | −0.37 | −0.510 | 1.021 |
| ATOM | 25 | P33 | UDP | 2 | 1.793 | −2.165 | −6.202 | −0.62 | +1.51 | +1.210 | 1.021 |
| ATOM | 26 | O36 | UDP | 2 | 1.032 | −2.171 | −4.925 | −0.38 | −2.06 | −0.850 | 1.021 |
| ATOM | 27 | O35 | UDP | 2 | 1.275 | −1.418 | −7.378 | −0.33 | −0.35 | −0.255 | 1.021 |
| ATOM | 28 | O34 | UDP | 2 | 3.288 | −1.589 | −5.999 | −0.16 | −1.10 | −0.850 | 1.021 |
| TER | | | | | | | | | | | |

ENDMDL

| | |
|---|---|
| MODEL | 4 |

| USER | Run = 4 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure = 1.074 A | | | | | | | | | | |
| USER | | | | | | | | | | | |
| USER | Estimated Free Energy of Binding | | | | | = −8.77 kcal/mol [= (1) + (3)] | | | | | |
| USER | Estimated Inhibition Constant, Ki | | | | | = +3.72e−07 [Temperature = 298.15 K] | | | | | |
| USER | | | | | | | | | | | |
| USER | Final Docked Energy | | | | | = −11.25 kcal/mol [= (1) + (2)] | | | | | |
| USER | | | | | | | | | | | |
| USER | (1) Final Intermolecular Energy | | | | | = −11.26 kcal/mol | | | | | |
| USER | (2) Final Internal Energy of Ligand | | | | | = +0.01 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.899388 −8.052323 −5.406975 | | | | | | | | | | |
| USER | NEWDPF quat0 −0.333985 0.729098 −0.597386 5.807816 | | | | | | | | | | |
| USER | NEWDPF ndihe8 | | | | | | | | | | |
| USER | NEWDPF dihe0 62.50 95.79 −47.55 −29.58 8.74 −99.23 −46.88 18.99 | | | | | | | | | | |
| USER | | | | | | | | | | | |
| USER | | | | Rank | x | y | z | vdW | Elec | q | RMS |
| ATOM | 1 | C19 | UDP | 2 | −1.113 | −9.448 | −6.012 | −0.49 | +0.10 | +0.174 | 1.074 |
| ATOM | 2 | C1 | UDP | 2 | −1.987 | −8.308 | −5.488 | −0.37 | +0.06 | +0.113 | 1.074 |
| ATOM | 3 | C3 | UDP | 2 | −1.248 | −7.089 | −6.087 | −0.37 | +0.02 | +0.113 | 1.074 |
| ATOM | 4 | O16 | UDP | 2 | 0.204 | −8.947 | −5.816 | −0.03 | −0.12 | −0.227 | 1.074 |
| ATOM | 5 | C14 | UDP | 2 | 0.218 | −7.519 | −6.089 | −0.43 | +0.04 | +0.113 | 1.074 |
| ATOM | 6 | N10 | UDP | 2 | −1.201 | −10.692 | −5.236 | −0.33 | +0.03 | +0.039 | 1.074 |
| ATOM | 7 | C11 | UDP | 2 | −1.282 | −11.886 | −5.895 | −0.66 | −0.06 | −0.073 | 1.074 |
| ATOM | 8 | O17 | UDP | 2 | −1.281 | −11.950 | −7.127 | −0.20 | −0.02 | −0.026 | 1.074 |
| ATOM | 9 | N12 | UDP | 2 | −1.347 | −13.012 | −5.114 | −0.48 | −0.21 | −0.292 | 1.074 |
| ATOM | 10 | H12 | UDP | 2 | −1.395 | −13.970 | −5.632 | −0.26 | +0.17 | +0.393 | 1.074 |
| ATOM | 11 | C9 | UDP | 2 | −1.359 | −13.047 | −3.719 | −0.66 | −0.02 | −0.023 | 1.074 |
| ATOM | 12 | O18 | UDP | 2 | −1.415 | −14.147 | −3.167 | −0.11 | −0.02 | −0.026 | 1.074 |
| ATOM | 13 | C7 | UDP | 2 | −1.279 | −11.767 | −3.089 | −0.47 | −0.00 | +0.000 | 1.074 |
| ATOM | 14 | C5 | UDP | 2 | −1.220 | −10.660 | −3.844 | −0.18 | −0.03 | −0.050 | 1.074 |
| ATOM | 15 | O21 | UDP | 2 | −3.285 | −8.392 | −6.027 | −0.24 | −0.60 | −0.537 | 1.074 |
| ATOM | 16 | H21 | UDP | 2 | −3.966 | −8.030 | −5.343 | +0.08 | +0.62 | +0.424 | 1.074 |
| ATOM | 17 | O23 | UDP | 2 | −1.707 | −6.941 | −7.434 | −0.08 | −0.09 | −0.537 | 1.074 |
| ATOM | 18 | H23 | UDP | 2 | −1.638 | −5.954 | −7.714 | −0.23 | −0.30 | +0.424 | 1.074 |
| ATOM | 19 | O25 | UDP | 2 | 1.047 | −6.826 | −5.006 | −0.37 | +0.04 | +0.113 | 1.074 |
| ATOM | 20 | O28 | UDP | 2 | 1.843 | −5.839 | −5.636 | +0.07 | −0.18 | −0.368 | 1.074 |
| ATOM | 21 | P39 | UDP | 2 | 2.633 | −4.580 | −5.104 | −0.35 | +0.96 | +1.210 | 1.074 |
| ATOM | 22 | O30 | UDP | 2 | 2.058 | −4.212 | −3.779 | +0.11 | −0.65 | −0.850 | 1.074 |
| ATOM | 23 | O31 | UDP | 2 | 4.098 | −4.868 | −5.184 | −0.03 | −1.02 | −0.850 | 1.074 |
| ATOM | 24 | O32 | UDP | 2 | 2.098 | −3.580 | −6.223 | +0.02 | −0.41 | −0.510 | 1.074 |
| ATOM | 25 | P33 | UDP | 2 | 1.843 | −2.001 | −6.246 | −0.63 | +1.57 | +1.210 | 1.074 |
| ATOM | 26 | O36 | UDP | 2 | 1.032 | −1.829 | −5.012 | −0.48 | −2.10 | −0.850 | 1.074 |
| ATOM | 27 | O35 | UDP | 2 | 1.350 | −1.476 | −7.546 | −0.29 | −0.33 | −0.255 | 1.074 |
| ATOM | 28 | O34 | UDP | 2 | 3.310 | −1.344 | −6.088 | −0.15 | −1.13 | −0.850 | 1.074 |
| TER | | | | | | | | | | | |

ENDMDL

| | |
|---|---|
| MODEL | 15 |

| USER | Run = 15 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure = 1.183 A | | | | | | | | | | |
| USER | | | | | | | | | | | |
| USER | Estimated Free Energy of Binding | | | | | = −8.34 kcal/mol [= (1) + (3)] | | | | | |
| USER | Estimated Inhibition Constant, Ki | | | | | = +7.73e−07 [Temperature = 298.15 K] | | | | | |
| USER | | | | | | | | | | | |
| USER | Final Docked Energy | | | | | = −11.22 kcal/mol [= (1) + (2)] | | | | | |
| USER | | | | | | | | | | | |
| USER | (1) Final Intermolecular Energy | | | | | = −10.83 kcal/mol | | | | | |
| USER | (2) Final Internal Energy of Ligand | | | | | = −0.39 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWPDF move ligand.pdbq | | | | | | | | | | |
| USER | NEWDPF about −1.222000 −8.102000 −5.172000 | | | | | | | | | | |
| USER | NEWDPF tran0 −1.166887 −8.068167 −5.148550 | | | | | | | | | | |
| USER | NEWDPF quat0 −0.504662 −0.722276 0.472898 −0.546298 | | | | | | | | | | |
| USER | NEWDPF ndihe8 | | | | | | | | | | |
| USER | NEWDPF dihe0 19.41 109.97 23.82 −21.43 4.01 −150.51 −49.03 176.96 | | | | | | | | | | |
| USER | | | | | | | | | | | |
| USER | | | | Rank | x | y | z | vdW | Elec | q | RMS |
| ATOM | 1 | C19 | UDP | 2 | −1.261 | −9.448 | −5.818 | −0.46 | +0.10 | +0.174 | 1.183 |
| ATOM | 2 | C1 | UDP | 2 | −2.230 | −8.380 | −5.313 | −0.36 | +0.07 | +0.113 | 1.183 |
| ATOM | 3 | C3 | UDP | 2 | −1.524 | −7.098 | −5.814 | −0.38 | +0.03 | +0.113 | 1.183 |
| ATOM | 4 | O16 | UDP | 2 | 0.010 | −8.884 | −5.514 | +0.02 | −0.12 | −0.227 | 1.183 |
| ATOM | 5 | C14 | UDP | 2 | −0.039 | −7.447 | −5.732 | −0.39 | +0.04 | +0.113 | 1.183 |
| ATOM | 6 | N10 | UDP | 2 | −1.328 | −10.725 | −5.095 | −0.32 | +0.03 | +0.039 | 1.183 |
| ATOM | 7 | C11 | UDP | 2 | −1.395 | −11.891 | −5.806 | −0.65 | −0.06 | −0.073 | 1.183 |
| ATOM | 8 | O17 | UDP | 2 | −1.399 | −11.902 | −7.039 | −0.24 | −0.02 | −0.026 | 1.183 |
| ATOM | 9 | N12 | UDP | 2 | −1.442 | −13.050 | −5.073 | −0.46 | −0.20 | −0.292 | 1.183 |
| ATOM | 10 | H12 | UDP | 2 | −1.479 | −13.985 | −5.632 | −0.27 | +0.15 | +0.393 | 1.183 |
| ATOM | 11 | C9 | UDP | 2 | −1.448 | −13.146 | −3.681 | −0.65 | −0.02 | −0.023 | 1.183 |
| ATOM | 12 | O18 | UDP | 2 | −1.487 | −14.269 | −3.177 | −0.11 | −0.02 | −0.026 | 1.183 |
| ATOM | 13 | C7 | UDP | 2 | −1.384 | −11.893 | −2.997 | −0.50 | +0.00 | +0.000 | 1.183 |
| ATOM | 14 | C5 | UDP | 2 | −1.343 | −10.753 | −3.704 | −0.23 | −0.03 | −0.050 | 1.183 |
| ATOM | 15 | O21 | UDP | 2 | −3.483 | −8.512 | −5.942 | −0.22 | −0.61 | −0.537 | 1.183 |
| ATOM | 16 | H21 | UDP | 2 | −4.158 | −8.925 | −5.282 | +0.07 | +0.47 | +0.424 | 1.183 |
| ATOM | 17 | O23 | UDP | 2 | −1.901 | −6.921 | −7.184 | −0.11 | −0.12 | −0.537 | 1.183 |
| ATOM | 18 | H23 | UDP | 2 | −1.646 | −5.973 | −7.490 | −0.22 | −0.34 | +0.424 | 1.183 |
| ATOM | 19 | C25 | UDP | 2 | 0.676 | −6.755 | −4.571 | −0.33 | +0.04 | +0.113 | 1.183 |
| ATOM | 20 | O28 | UDP | 2 | 1.493 | −5.734 | −5.113 | +0.09 | −0.17 | −0.368 | 1.183 |
| ATOM | 21 | P39 | UDP | 2 | 1.440 | −4.158 | −5.055 | −0.30 | +0.88 | +1.210 | 1.183 |
| ATOM | 22 | O30 | UDP | 2 | 0.110 | −3.769 | −4.506 | −0.25 | −1.13 | −0.850 | 1.183 |
| ATOM | 23 | O31 | UDP | 2 | 2.666 | −3.666 | −4.353 | +0.08 | −0.77 | −0.850 | 1.183 |
| ATOM | 24 | O32 | UDP | 2 | 1.458 | −3.945 | −6.634 | +0.02 | −0.23 | −0.510 | 1.183 |
| ATOM | 25 | P33 | UDP | 2 | 2.229 | −2.922 | −7.591 | −0.53 | +1.10 | +1.210 | 1.183 |
| ATOM | 26 | O36 | UDP | 2 | 1.918 | −1.628 | −6.929 | −0.23 | −1.19 | −0.850 | 1.183 |
| ATOM | 27 | O35 | UDP | 2 | 1.955 | −3.108 | −9.039 | −0.13 | −0.18 | −0.255 | 1.183 |
| ATOM | 28 | O34 | UDP | 2 | 3.790 | −3.308 | −7.434 | −0.23 | −1.26 | −0.850 | 1.183 |
| TER | | | | | | | | | | | |

ENDMDL

| | |
|---|---|
| MODEL | 35 |

| USER | Run = 35 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure = 1.006 A | | | | | | | | | | |
| USER | | | | | | | | | | | |
| USER | Estimated Free Energy of Binding | | | | | = −8.67 kcal/mol [= (1) + (3)] | | | | | |
| USER | | | | | | | | | | | |
| USER | Estimated Inhibition Constant, Ki | | | | | = +4.43e−07 [Temperature = 298.15 K] | | | | | |
| USER | | | | | | | | | | | |
| USER | Final Docked Energy | | | | | = −11.19 kcal/mol [= (1) + (2)] | | | | | |
| USER | | | | | | | | | | | |
| USER | (1) Final Intermolecular Energy | | | | | = −11.16 kcal/mol | | | | | |
| USER | (2) Final Internal Energy of Ligand | | | | | = −0.03 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.396748 −8.114173 −5.430786 | | | | | | | | | | |
| USER | NEWDPF quat0 0.587115 −0.641976 −0.493116 −4.067627 | | | | | | | | | | |
| USER | NEWDPF ndihe8 | | | | | | | | | | |
| USER | NEWDPF dihe0 54.84 112.56 3.96 −111.74 3.92 −86.87 −1.81 133.94 | | | | | | | | | | |
| USER | | | | | | | | | | | |
| USER | | | | Rank | x | y | z | vdW | Elec | q | RMS |
| ATOM | 1 | C19 | UDP | 2 | −1.460 | −9.526 | −6.032 | −0.48 | +0.11 | +0.174 | 1.006 |
| ATOM | 2 | C1 | UDP | 2 | −2.452 | −8.474 | −5.538 | −0.38 | +0.08 | +0.113 | 1.006 |
| ATOM | 3 | C3 | UDP | 2 | −1.818 | −7.191 | −6.125 | −0.41 | +0.04 | +0.113 | 1.006 |
| ATOM | 4 | O16 | UDP | 2 | −0.202 | −8.900 | −5.805 | +0.00 | −0.12 | −0.227 | 1.006 |
| ATOM | 5 | C14 | UDP | 2 | −0.318 | −7.478 | −6.086 | −0.41 | +0.03 | +0.113 | 1.006 |
| ATOM | 6 | N10 | UDP | 2 | −1.448 | −10.770 | −5.250 | −0.32 | +0.03 | +0.039 | 1.006 |
| ATOM | 7 | C11 | UDP | 2 | −1.496 | −11.969 | −5.904 | −0.63 | −0.06 | −0.073 | 1.006 |
| ATOM | 8 | O17 | UDP | 2 | −1.547 | −12.039 | −7.134 | −0.21 | −0.02 | −0.026 | 1.006 |
| ATOM | 9 | N12 | UDP | 2 | −1.468 | −13.094 | −5.118 | −0.44 | −0.20 | −0.292 | 1.006 |
| ATOM | 10 | H12 | UDP | 2 | −1.489 | −14.055 | −5.632 | −0.27 | +0.15 | +0.393 | 1.006 |
| ATOM | 11 | C9 | UDP | 2 | −1.416 | −13.124 | −3.724 | −0.66 | −0.02 | −0.023 | 1.006 |
| ATOM | 12 | O18 | UDP | 2 | −1.390 | −14.223 | −3.168 | −0.11 | −0.02 | −0.026 | 1.006 |
| ATOM | 13 | C7 | UDP | 2 | −1.375 | −11.839 | −3.101 | −0.49 | +0.00 | +0.000 | 1.006 |
| ATOM | 14 | C5 | UDP | 2 | −1.407 | −10.733 | −3.860 | −0.30 | −0.03 | −0.050 | 1.006 |
| ATOM | 15 | O21 | UDP | 2 | −3.722 | −8.684 | −6.111 | −0.19 | −0.62 | −0.537 | 1.006 |
| ATOM | 16 | H21 | UDP | 2 | −4.451 | −8.509 | −5.404 | +0.07 | +0.58 | +0.424 | 1.006 |
| ATOM | 17 | O23 | UDP | 2 | −2.253 | −7.093 | −7.486 | −0.25 | −0.24 | −0.537 | 1.006 |
| ATOM | 18 | H23 | UDP | 2 | −2.010 | −6.166 | −7.858 | −0.21 | −0.07 | +0.424 | 1.006 |
| ATOM | 19 | C25 | UDP | 2 | 0.412 | −6.705 | −4.987 | −0.32 | +0.03 | +0.113 | 1.006 |
| ATOM | 20 | O28 | UDP | 2 | 0.218 | −5.324 | −5.229 | +0.12 | −0.04 | −0.368 | 1.006 |
| ATOM | 21 | P39 | UDP | 2 | 0.614 | −4.018 | −4.436 | −0.20 | +1.14 | +1.210 | 1.006 |
| ATOM | 22 | O30 | UDP | 2 | −0.609 | −3.519 | −3.745 | −0.24 | −1.41 | −0.850 | 1.006 |
| ATOM | 23 | O31 | UDP | 2 | 1.824 | −4.311 | −3.608 | +0.12 | −0.61 | −0.850 | 1.006 |
| ATOM | 24 | O32 | UDP | 2 | 0.902 | −3.133 | −5.730 | −0.24 | −0.55 | −0.510 | 1.006 |
| ATOM | 25 | P33 | UDP | 2 | 1.783 | −1.824 | −5.984 | −0.64 | +1.73 | +1.210 | 1.006 |
| ATOM | 26 | O36 | UDP | 2 | 1.298 | −0.941 | −4.891 | −0.73 | −1.72 | −0.850 | 1.006 |
| ATOM | 27 | O35 | UDP | 2 | 1.781 | −1.344 | −7.390 | −0.06 | −0.39 | −0.255 | 1.006 |
| ATOM | 28 | O34 | UDP | 2 | 3.308 | −2.296 | −5.733 | −0.07 | −1.02 | −0.850 | 1.006 |
| TER | | | | | | | | | | | |

ENDMDL

| | |
|---|---|
| MODEL | 27 |

| USER | Run = 27 | | | | | | | | | | |
|---|---|---|---|---|---|---|---|---|---|---|---|
| USER | Cluster Rank = 2 | | | | | | | | | | |
| USER | Number of conformations in this cluster = 15 | | | | | | | | | | |
| USER | | | | | | | | | | | |
| USER | RMSD from reference structure = 0.990 A | | | | | | | | | | |
| USER | | | | | | | | | | | |
| USER | Estimated Free Energy of Binding | | | | | = −8.57 kcal/mol [= (1) + (3)] | | | | | |
| USER | Estimated Inhibition Constant, Ki | | | | | = +5.23e−07 [Temperature = 298.15 K] | | | | | |
| USER | | | | | | | | | | | |
| USER | Final Docked Energy | | | | | = −11.01 kcal/mol [= (1) + (2)] | | | | | |
| USER | | | | | | | | | | | |
| USER | (1) Final Intermolecular Energy | | | | | = −11.06 kcal/mol | | | | | |
| USER | (2) Final Internal Energy of Ligand | | | | | = +0.05 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.188063 −8.021883 −5.111572 | | | | | | | | | | |
| USER | NEWDPF quat0 0.342713 −0.664948 −0.663620 1.279332 | | | | | | | | | | |
| USER | NEWDPF ndihe8 | | | | | | | | | | |
| USER | NEWDPF dihe0 23.90 111.73 −116.30 −138.88 8.77 −105.59 3.54 3.95 | | | | | | | | | | |
| USER | | | | | | | | | | | |
| USER | | | | Rank | x | y | z | vdW | Elec | q | RMS |
| ATOM | 1 | C19 | UDP | 2 | −1.282 | −9.398 | −5.786 | −0.45 | +0.10 | +0.174 | 0.990 |
| ATOM | 2 | C1 | UDP | 2 | −2.251 | −8.322 | −5.300 | −0.36 | +0.07 | +0.113 | 0.990 |
| ATOM | 3 | C3 | UDP | 2 | −1.521 | −7.047 | −5.782 | −0.38 | +0.03 | +0.113 | 0.990 |
| ATOM | 4 | O16 | UDP | 2 | −0.012 | −8.849 | −5.453 | +0.02 | −0.12 | −0.227 | 0.990 |
| ATOM | 5 | C14 | UDP | 2 | −0.041 | −7.411 | −5.669 | −0.38 | +0.04 | +0.113 | 0.990 |
| ATOM | 6 | N10 | UDP | 2 | −1.378 | −10.677 | −5.069 | −0.31 | +0.03 | +0.039 | 0.990 |
| ATOM | 7 | C11 | UDP | 2 | −1.343 | −11.845 | −5.779 | −0.65 | −0.06 | −0.073 | 0.990 |
| ATOM | 8 | O17 | UDP | 2 | −1.230 | −11.856 | −7.007 | −0.24 | −0.02 | −0.026 | 0.990 |
| ATOM | 9 | N12 | UDP | 2 | −1.423 | −13.005 | −5.050 | −0.47 | −0.21 | −0.292 | 0.990 |
| ATOM | 10 | H12 | UDP | 2 | −1.379 | −13.941 | −5.607 | −0.26 | +0.18 | +0.393 | 0.990 |
| ATOM | 11 | C9 | UDP | 2 | −1.557 | −13.099 | −3.665 | −0.63 | −0.02 | −0.023 | 0.990 |
| ATOM | 12 | O18 | UDP | 2 | −1.609 | −14.223 | −3.162 | −0.10 | −0.02 | −0.026 | 0.990 |
| ATOM | 13 | C7 | UDP | 2 | −1.595 | −11.845 | −2.982 | −0.49 | +0.00 | +0.000 | 0.990 |
| ATOM | 14 | C5 | UDP | 2 | −1.522 | −10.705 | −3.686 | −0.34 | −0.03 | −0.050 | 0.990 |
| ATOM | 15 | O21 | UDP | 2 | −3.491 | −8.439 | −5.956 | −0.21 | −0.64 | −0.537 | 0.990 |
| ATOM | 16 | H21 | UDP | 2 | −4.202 | −8.777 | −5.291 | +0.07 | +0.49 | +0.424 | 0.990 |
| ATOM | 17 | O23 | UDP | 2 | −1.866 | −6.862 | −7.159 | −0.08 | −0.10 | −0.537 | 0.990 |
| ATOM | 18 | H23 | UDP | 2 | −1.568 | −5.926 | −7.465 | −0.21 | −0.39 | +0.424 | 0.990 |
| ATOM | 19 | C25 | UDP | 2 | 0.655 | −6.730 | −4.490 | −0.32 | +0.04 | +0.113 | 0.990 |
| ATOM | 20 | O28 | UDP | 2 | 0.437 | −5.336 | −4.607 | +0.09 | −0.13 | −0.368 | 0.990 |
| ATOM | 21 | P39 | UDP | 2 | 0.444 | −4.147 | −3.569 | −0.23 | +1.01 | +1.210 | 0.990 |
| ATOM | 22 | O30 | UDP | 2 | −0.817 | −3.374 | −3.757 | −0.23 | −1.59 | −0.850 | 0.990 |
| ATOM | 23 | O31 | UDP | 2 | 0.739 | −4.700 | −2.211 | +0.16 | −0.39 | −0.850 | 0.990 |
| ATOM | 24 | O32 | UDP | 2 | 1.661 | −3.366 | −4.238 | −0.01 | −0.54 | −0.510 | 0.990 |
| ATOM | 25 | P33 | UDP | 2 | 1.764 | −2.353 | −5.471 | −0.52 | +1.61 | +1.210 | 0.990 |
| ATOM | 26 | O36 | UDP | 2 | 1.038 | −3.113 | −6.522 | −0.06 | −0.54 | −0.850 | 0.990 |
| ATOM | 27 | O35 | UDP | 2 | 3.132 | −1.837 | −5.731 | −0.12 | −0.31 | −0.255 | 0.990 |
| ATOM | 28 | O34 | UDP | 2 | 0.934 | −1.047 | −5.003 | −0.74 | −2.12 | −0.850 | 0.990 |
| TER | | | | | | | | | | | |

ENDMDL

| | |
|---|---|
| MODEL | 21 |

| USER | Run = 21 | | | | | | | | | | |
|---|---|---|---|---|---|---|---|---|---|---|---|
| USER | Cluster Rank = 3 | | | | | | | | | | |
| USER | Number of conformations in this cluster = 1 | | | | | | | | | | |
| USER | | | | | | | | | | | |
| USER | RMSD from reference structure = 2.539 A | | | | | | | | | | |
| USER | | | | | | | | | | | |
| USER | Estimated Free Energy of Binding | | | | | = −10.43 kcal/mol [= (1) + (3)] | | | | | |
| USER | Estimated Inhibition Constant, Ki | | | | | = +2.27e−08 [Temperature = 298.15 K] | | | | | |
| USER | | | | | | | | | | | |
| USER | Final Docked Energy | | | | | = −12.55 kcal/mol [= (1) + (2)] | | | | | |
| USER | | | | | | | | | | | |
| USER | (1) Final Intermolecular Energy | | | | | = −12.92 kcal/mol | | | | | |
| USER | (2) Final Internal Energy of Ligand | | | | | = +0.37 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 1.647507 −6.281227 −6.685684 | | | | | | | | | | |
| USER | NEWDPF quat0 −0.076797 −0.823729 −0.561758 40.378962 | | | | | | | | | | |
| USER | NEWDPF ndihe8 | | | | | | | | | | |
| USER | NEWDPF dihe0 43.50 29.10 −4.82 148.96 178.20 −175.22 −31.58 155.47 | | | | | | | | | | |
| USER | | | | | | | | | | | |
| USER | | | | Rank | x | y | z | vdW | Elec | q | RMS |
| ATOM | 1 | C19 | UDP | 3 | 1.407 | −7.635 | −7.369 | −0.45 | +0.08 | +0.174 | 2.539 |
| ATOM | 2 | C1 | UDP | 3 | 0.807 | −6.231 | −7.424 | −0.40 | +0.02 | +0.113 | 2.539 |
| ATOM | 3 | C3 | UDP | 3 | 2.093 | −5.373 | −7.385 | −0.51 | +0.06 | +0.113 | 2.539 |
| ATOM | 4 | O16 | UDP | 3 | 2.427 | −7.500 | −6.387 | −0.05 | −0.12 | −0.227 | 2.539 |
| ATOM | 5 | C14 | UDP | 3 | 3.048 | −6.192 | −6.519 | −0.52 | +0.08 | +0.113 | 2.539 |
| ATOM | 6 | N10 | UDP | 3 | 0.492 | −8.672 | −6.874 | −0.40 | +0.02 | +0.039 | 2.539 |
| ATOM | 7 | C11 | UDP | 3 | −0.131 | −8.492 | −5.670 | −0.44 | −0.03 | −0.073 | 2.539 |
| ATOM | 8 | O17 | UDP | 3 | 0.051 | −7.482 | −4.988 | +0.06 | −0.01 | −0.026 | 2.539 |
| ATOM | 9 | N12 | UDP | 3 | −0.984 | −9.490 | −5.270 | −0.25 | −0.17 | −0.292 | 2.539 |
| ATOM | 10 | H12 | UDP | 3 | −1.498 | −9.347 | −4.319 | +0.09 | +0.21 | +0.393 | 2.539 |
| ATOM | 11 | C9 | UDP | 3 | −1.255 | −10.670 | −5.963 | −0.59 | −0.02 | −0.023 | 2.539 |
| ATOM | 12 | O18 | UDP | 3 | −2.055 | −11.464 | −5.466 | −0.25 | −0.02 | −0.026 | 2.539 |
| ATOM | 13 | C7 | UDP | 3 | −0.568 | −10.801 | −7.209 | −0.75 | +0.00 | +0.000 | 2.539 |
| ATOM | 14 | C5 | UDP | 3 | 0.271 | −9.834 | −7.607 | −0.76 | −0.04 | −0.050 | 2.539 |
| ATOM | 15 | O21 | UDP | 3 | 0.134 | −6.028 | −8.645 | −0.10 | −0.03 | −0.537 | 2.539 |
| ATOM | 16 | H21 | UDP | 3 | −0.814 | −5.670 | −8.460 | −0.27 | −0.23 | +0.424 | 2.539 |
| ATOM | 17 | O23 | UDP | 3 | 2.595 | −5.308 | −8.724 | −0.12 | −0.38 | −0.537 | 2.539 |
| ATOM | 18 | H23 | UDP | 3 | 2.131 | −4.541 | −9.228 | +0.06 | +0.26 | +0.424 | 2.539 |
| ATOM | 19 | C25 | UDP | 3 | 3.243 | −5.605 | −5.120 | −0.47 | +0.09 | +0.113 | 2.539 |
| ATOM | 20 | O28 | UDP | 3 | 4.328 | −4.698 | −5.179 | −0.06 | −0.48 | −0.368 | 2.539 |
| ATOM | 21 | P39 | UDP | 3 | 4.443 | −3.166 | −5.541 | −0.59 | +1.89 | +1.210 | 2.539 |
| ATOM | 22 | O30 | UDP | 3 | 5.699 | −2.648 | −4.927 | −0.35 | −1.75 | −0.850 | 2.539 |
| ATOM | 23 | O31 | UDP | 3 | 4.260 | −3.010 | −7.017 | −0.26 | −1.57 | −0.850 | 2.539 |
| ATOM | 24 | O32 | UDP | 3 | 3.188 | −2.708 | −4.672 | +0.01 | −0.53 | −0.510 | 2.539 |
| ATOM | 25 | P33 | UDP | 3 | 2.329 | −1.361 | −4.619 | −0.62 | +1.48 | +1.210 | 2.539 |
| ATOM | 26 | O36 | UDP | 3 | 3.406 | −0.344 | −4.491 | −0.19 | −0.80 | −0.850 | 2.539 |
| ATOM | 27 | O35 | UDP | 3 | 1.203 | −1.386 | −3.649 | −0.31 | −0.41 | −0.255 | 2.539 |
| ATOM | 28 | O34 | UDP | 3 | 1.603 | −1.277 | −6.061 | −0.51 | −1.53 | −0.850 | 2.539 |
| TER | | | | | | | | | | | |

ENDMDL

| | |
|---|---|
| MODEL | 6 |

| USER | Run = 6 | | | | | | | | | | |
|---|---|---|---|---|---|---|---|---|---|---|---|
| USER | Cluster Rank = 4 | | | | | | | | | | |
| USER | Number of conformations in this cluster = 1 | | | | | | | | | | |
| USER | | | | | | | | | | | |
| USER | RMSD from reference structure = 2.779 A | | | | | | | | | | |
| USER | | | | | | | | | | | |
| USER | Estimated Free Energy of Binding | | | | | = −9.74 kcal/mol [= (1) + (3)] | | | | | |
| USER | Estimated Inhibition Constant, Ki | | | | | = +7.20e−08 [Temperature = 298.15 K] | | | | | |
| USER | | | | | | | | | | | |
| USER | Final Docked Energy | | | | | = −12.08 kcal/mol [= (1) + (2)] | | | | | |
| USER | | | | | | | | | | | |
| USER | (1) Final Intermolecular Energy | | | | | = −12.23 kcal/mol | | | | | |
| USER | (2) Final Internal Energy of Ligand | | | | | = +0.15 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 0.701191 −7.302628 −6.976651 | | | | | | | | | | |
| USER | NEWDPF quat0 0.590050 0.790148 0.165852 −159.3851956 | | | | | | | | | | |
| USER | NEWDPF ndihe8 | | | | | | | | | | |
| USER | NEWDPF dihe0 28.94 37.56 35.34 133.90 −30.75 −33.69 1.16 39.08 | | | | | | | | | | |
| USER | | | | | | | | | | | |
| USER | | | | Rank | x | y | z | vdW | Elec | q | RMS |
| ATOM | 1 | C19 | UDP | 4 | −0.547 | −8.056 | −6.494 | −0.47 | +0.07 | +0.174 | 2.779 |
| ATOM | 2 | C1 | UDP | 4 | 0.690 | −8.363 | −7.337 | −0.65 | +0.06 | +0.113 | 2.779 |
| ATOM | 3 | C3 | UDP | 4 | 1.782 | −7.651 | −6.505 | −0.51 | +0.05 | +0.113 | 2.779 |
| ATOM | 4 | O16 | UDP | 4 | −0.358 | −6.688 | −6.149 | +0.07 | −0.01 | −0.227 | 2.779 |
| ATOM | 5 | C14 | UDP | 4 | 1.056 | −6.447 | −5.909 | −0.38 | +0.04 | +0.113 | 2.779 |
| ATOM | 6 | N10 | UDP | 4 | −1.819 | −8.124 | −7.226 | −0.34 | +0.02 | +0.039 | 2.779 |
| ATOM | 7 | C11 | UDP | 4 | −2.570 | −9.263 | −7.154 | −0.59 | −0.05 | −0.073 | 2.779 |
| ATOM | 8 | O17 | UDP | 4 | −2.212 | −10.238 | −6.489 | −0.07 | −0.02 | −0.026 | 2.779 |
| ATOM | 9 | N12 | UDP | 4 | −3.754 | −9.259 | −7.849 | −0.52 | −0.21 | −0.292 | 2.779 |
| ATOM | 10 | H12 | UDP | 4 | 4.367 | −10.158 | −7.784 | +0.13 | +0.32 | +0.393 | 2.779 |
| ATOM | 11 | C9 | UDP | 4 | −4.245 | −8.214 | −8.632 | −0.92 | −0.02 | −0.023 | 2.779 |
| ATOM | 12 | O18 | UDP | 4 | −5.334 | −8.368 | −9.188 | −0.45 | −0.02 | −0.026 | 2.779 |
| ATOM | 13 | C7 | UDP | 4 | −3.411 | −7.055 | −8.672 | −0.57 | +0.00 | +0.000 | 2.779 |
| ATOM | 14 | C5 | UDP | 4 | −2.250 | −7.051 | −8.000 | −0.58 | −0.02 | −0.050 | 2.779 |
| ATOM | 15 | O21 | UDP | 4 | 0.937 | −9.749 | −7.365 | −0.20 | −0.39 | −0.537 | 2.779 |
| ATOM | 16 | H21 | UDP | 4 | 0.812 | −10.102 | −8.325 | +0.01 | +0.43 | +0.424 | 2.779 |
| ATOM | 17 | O23 | UDP | 4 | 2.162 | −8.550 | −5.458 | +0.05 | −0.27 | −0.537 | 2.779 |
| ATOM | 18 | H23 | UDP | 4 | 2.979 | −9.099 | −5.756 | −0.07 | +0.03 | +0.424 | 2.779 |
| ATOM | 19 | C25 | UDP | 4 | 1.442 | −5.125 | −6.574 | −0.33 | +0.03 | +0.113 | 2.779 |
| ATOM | 20 | O28 | UDP | 4 | 2.845 | −4.979 | −6.465 | +0.04 | −0.29 | −0.368 | 2.779 |
| ATOM | 21 | P39 | UDP | 4 | 3.781 | −3.797 | −5.998 | −0.52 | +1.56 | +1.210 | 2.779 |
| ATOM | 22 | O30 | UDP | 4 | 4.930 | −4.392 | −5.259 | −0.09 | −1.45 | −0.850 | 2.779 |
| ATOM | 23 | O31 | UDP | 4 | 4.073 | −2.926 | −7.178 | −0.28 | −1.44 | −0.850 | 2.779 |
| ATOM | 24 | O32 | UDP | 4 | 2.738 | −3.175 | −4.965 | +0.05 | −0.51 | −0.510 | 2.779 |
| ATOM | 25 | P33 | UDP | 4 | 2.122 | −1.711 | −4.782 | −0.58 | +1.60 | +1.210 | 2.779 |
| ATOM | 26 | O36 | UDP | 4 | 3.357 | −0.883 | −4.784 | −0.19 | −0.91 | −0.850 | 2.779 |
| ATOM | 27 | O35 | UDP | 4 | 1.141 | −1.590 | −3.673 | −0.31 | −0.44 | −0.255 | 2.779 |
| ATOM | 28 | O34 | UDP | 4 | 1.241 | −1.464 | −6.114 | −0.62 | −1.51 | −0.850 | 2.779 |
| TER | | | | | | | | | | | |

ENDMDL

| | |
|---|---|
| MODEL | 50 |

| USER | Run = 50 | | | | | | | | | | |
|---|---|---|---|---|---|---|---|---|---|---|---|
| USER | Cluster Rank = 50 | | | | | | | | | | |
| USER | Number of conformations in this cluster = 1 | | | | | | | | | | |
| USER | | | | | | | | | | | |
| USER | RMSD from reference structure = 2.809 A | | | | | | | | | | |
| USER | | | | | | | | | | | |
| USER | Estimated Free Energy of Binding | | | | | = −9.46 kcal/mol [= (1) + (3)] | | | | | |
| USER | Estimated Inhibition Constant, Ki | | | | | = +1.17e−07 [Temperature = 298.15 K] | | | | | |
| USER | | | | | | | | | | | |
| USER | Final Docked Energy | | | | | = −12.07 kcal/mol [= (1) + (2)] | | | | | |
| USER | | | | | | | | | | | |
| USER | (1) Final Intermolecular Energy | | | | | = −11.95 kcal/mol | | | | | |
| USER | (2) Final Internal Energy of Ligand | | | | | = −0.12 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 2.180910 −5.980346 −6.489515 | | | | | | | | | | |
| USER | NEWDPF quat0 0.302605 0.855562 0.420053 −85.787078 | | | | | | | | | | |
| USER | NEWDPF ndihe8 | | | | | | | | | | |
| USER | NEWDPF dihe0 13.75 14.76 57.22 −179.75 −2.85 −156.78 −10.65 93.61 | | | | | | | | | | |
| USER | | | | | | | | | | | |
| USER | | | | Rank | x | y | z | vdW | Elec | q | RMS |
| ATOM | 1 | C19 | UDP | 5 | 1.744 | −7.426 | −6.770 | −0.49 | +0.08 | +0.174 | 2.809 |
| ATOM | 2 | C1 | UDP | 5 | 1.929 | −6.137 | −7.570 | −0.54 | +0.06 | +0.113 | 2.809 |
| ATOM | 3 | C3 | UDP | 5 | 3.255 | −5.618 | −6.966 | −0.54 | +0.10 | +0.113 | 2.809 |
| ATOM | 4 | O16 | UDP | 5 | 2.093 | −7.027 | −5.450 | −0.02 | −0.12 | −0.227 | 2.809 |
| ATOM | 5 | C14 | UDP | 5 | 3.196 | −6.082 | −5.511 | −0.51 | +0.09 | +0.113 | 2.809 |
| ATOM | 6 | N10 | UDP | 5 | 0.359 | −7.909 | −6.703 | −0.33 | +0.02 | +0.039 | 2.809 |
| ATOM | 7 | C11 | UDP | 5 | 0.078 | −9.188 | −7.097 | −0.67 | −0.04 | −0.073 | 2.809 |
| ATOM | 8 | O17 | UDP | 5 | 0.956 | −9.950 | −7.508 | −0.20 | −0.02 | −0.026 | 2.809 |
| ATOM | 9 | N12 | UDP | 5 | −1.232 | −9.584 | −6.997 | −0.33 | −0.18 | −0.292 | 2.809 |
| ATOM | 10 | H12 | UDP | 5 | −1.462 | −10.607 | −7.294 | +0.07 | +0.27 | +0.393 | 2.809 |
| ATOM | 11 | C9 | UDP | 5 | −2.297 | −8.801 | −6.552 | −0.49 | −0.02 | −0.023 | 2.809 |
| ATOM | 12 | O18 | UDP | 5 | −3.418 | −9.312 | −6.515 | −0.11 | −0.02 | −0.026 | 2.809 |
| ATOM | 13 | C7 | UDP | 5 | −1.940 | −7.474 | −6.160 | −0.44 | +0.00 | +0.000 | 2.809 |
| ATOM | 14 | C5 | UDP | 5 | −0.663 | −7.075 | −6.260 | −0.37 | −0.01 | −0.050 | 2.809 |
| ATOM | 15 | O21 | UDP | 5 | 2.107 | −6.423 | −8.937 | −0.09 | −0.37 | −0.537 | 2.809 |
| ATOM | 16 | H21 | UDP | 5 | 1.217 | −6.286 | −9.436 | +0.04 | +0.23 | +0.424 | 2.809 |
| ATOM | 17 | O23 | UDP | 5 | 4.318 | −6.297 | −7.643 | +0.08 | −0.59 | −0.537 | 2.809 |
| ATOM | 18 | H23 | UDP | 5 | 4.388 | −5.948 | −8.608 | +0.01 | +0.45 | +0.424 | 2.809 |
| ATOM | 19 | C25 | UDP | 5 | 2.914 | −4.943 | −4.530 | −0.38 | +0.09 | +0.113 | 2.809 |
| ATOM | 20 | O28 | UDP | 5 | 4.049 | −4.097 | −4.512 | −0.04 | −0.43 | −0.368 | 2.809 |
| ATOM | 21 | P39 | UDP | 5 | 4.348 | −2.670 | −5.117 | −0.66 | +1.67 | +1.210 | 2.809 |
| ATOM | 22 | O30 | UDP | 5 | 5.806 | −2.405 | −4.954 | −0.34 | −1.78 | −0.850 | 2.809 |
| ATOM | 23 | O31 | UDP | 5 | 3.766 | −2.603 | −6.492 | −0.13 | −1.20 | −0.850 | 2.809 |
| ATOM | 24 | O32 | UDP | 5 | 3.525 | −1.861 | −4.016 | −0.05 | −0.46 | −0.510 | 2.809 |
| ATOM | 25 | P33 | UDP | 5 | 2.098 | −1.141 | −4.029 | −0.59 | +1.34 | +1.210 | 2.809 |
| ATOM | 26 | O36 | UDP | 5 | 2.213 | −0.310 | −5.257 | −0.15 | −1.48 | −0.850 | 2.809 |
| ATOM | 27 | O35 | UDP | 5 | 1.712 | −0.517 | −2.737 | −0.03 | −0.15 | −0.255 | 2.809 |
| ATOM | 28 | O34 | UDP | 5 | 1.032 | −2.339 | −4.221 | −0.30 | −1.93 | −0.850 | 2.809 |
| TER | | | | | | | | | | | |

ENDMDL

| | |
|---|---|
| MODEL | 2 |

| USER | Run = 2 | | | | | | | | | | |
|---|---|---|---|---|---|---|---|---|---|---|---|
| USER | Cluster Rank = 2 | | | | | | | | | | |
| USER | Number of conformations in this cluster = 1 | | | | | | | | | | |
| USER | | | | | | | | | | | |
| USER | RMSD from reference structure = 2.094 A | | | | | | | | | | |
| USER | | | | | | | | | | | |
| USER | Estimated Free Energy of Binding | | | | | = −9.35 kcal/mol [= (1) + (3)] | | | | | |
| USER | Estimated Inhibition Constant, Ki | | | | | = +1.39e−07 [Temperature = 298.15 K] | | | | | |
| USER | | | | | | | | | | | |
| USER | Final Docked Energy | | | | | = −11.99 kcal/mol [= (1) + (2)] | | | | | |
| USER | | | | | | | | | | | |
| USER | (1) Final Intermolecular Energy | | | | | = −11.84 kcal/mol | | | | | |
| USER | (2) Final Internal Energy of Ligand | | | | | = −0.14 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 0.210183 −7.499820 −6.214437 | | | | | | | | | | |
| USER | NEWDPF quat0 −0.220355 0.763463 0.607097 −21.532182 | | | | | | | | | | |
| USER | NEWDPF ndihe8 | | | | | | | | | | |
| USER | NEWDPF dihe0 70.53 60.05 53.56 −28.78 −2.13 −55.53 −41.01 −66.06 | | | | | | | | | | |
| USER | | | | | | | | | | | |
| USER | | | | Rank | x | y | z | vdW | Elec | q | RMS |
| ATOM | 1 | C19 | UDP | 6 | 0.033 | −8.791 | −7.027 | −0.61 | +0.09 | +0.174 | 2.094 |
| ATOM | 2 | C1 | UDP | 6 | −0.798 | −7.550 | −6.700 | −0.47 | +0.03 | +0.113 | 2.094 |
| ATOM | 3 | C3 | UDP | 6 | 0.275 | −6.447 | −6.845 | −0.13 | +0.00 | +0.113 | 2.094 |
| ATOM | 4 | O16 | UDP | 6 | 1.245 | −8.549 | −6.323 | −0.10 | −0.11 | −0.227 | 2.094 |
| ATOM | 5 | O14 | UDP | 6 | 1.563 | −7.132 | −6.392 | −0.51 | +0.05 | +0.113 | 2.094 |
| ATOM | 6 | N1O | UDP | 6 | −0.510 | −10.047 | −6.494 | −0.40 | +0.03 | +0.039 | 2.094 |
| ATOM | 7 | C11 | UDP | 6 | −0.724 | −11.096 | −7.344 | −0.65 | −0.07 | −0.073 | 2.094 |
| ATOM | 8 | O17 | UDP | 6 | −0.472 | −11.019 | −8.549 | −0.42 | −0.03 | −0.026 | 2.094 |
| ATOM | 9 | N12 | UDP | 6 | −1.218 | −12.247 | −6.782 | −0.10 | −0.23 | −0.292 | 2.094 |
| ATOM | 10 | H12 | UDP | 6 | −1.376 | −13.093 | −7.450 | −0.10 | +0.00 | +0.393 | 2.094 |
| ATOM | 11 | C9 | UDP | 6 | −1.539 | −12.435 | −5.437 | −0.57 | −0.02 | −0.023 | 2.094 |
| ATOM | 12 | O18 | UDP | 6 | −1.968 | −13.536 | −5.091 | +0.10 | −0.02 | −0.026 | 2.094 |
| ATOM | 13 | C7 | UDP | 6 | −1.299 | −11.300 | −4.602 | −0.50 | +0.00 | +0.000 | 2.094 |
| ATOM | 14 | C5 | UDP | 6 | −0.820 | −10.169 | −5.143 | −0.30 | −0.03 | −0.050 | 2.094 |
| ATOM | 15 | O21 | UDP | 6 | −1.809 | −7.364 | −7.663 | −0.13 | −0.19 | −0.537 | 2.094 |
| ATOM | 16 | H21 | UDP | 6 | −2.576 | −6.812 | −7.254 | +0.01 | +0.25 | +0.424 | 2.094 |
| ATOM | 17 | O23 | UDP | 6 | 0.365 | −6.129 | −8.238 | 0.06 | −0.03 | −0.537 | 2.094 |
| ATOM | 18 | H23 | UDP | 6 | 0.079 | −5.153 | −8.387 | 0.18 | −0.19 | +0.424 | 2.094 |
| ATOM | 19 | C25 | UDP | 6 | 2.032 | −6.673 | −5.010 | −0.43 | +0.06 | +0.113 | 2.094 |
| ATOM | 20 | O28 | UDP | 6 | 3.044 | −5.701 | −5.197 | +0.05 | −0.29 | −0.368 | 2.094 |
| ATOM | 21 | P39 | UDP | 6 | 4.270 | −5.230 | −4.322 | −0.61 | +1.41 | +1.210 | 2.094 |
| ATOM | 22 | O30 | UDP | 6 | 4.013 | −5.652 | −2.916 | −0.19 | −0.95 | −0.850 | 2.094 |
| ATOM | 23 | O31 | UDP | 6 | 5.531 | −5.692 | −4.980 | +0.35 | −1.45 | −0.850 | 2.094 |
| ATOM | 24 | O32 | UDP | 6 | 3.981 | −3.670 | −4.480 | −0.03 | −0.59 | −0.510 | 2.094 |
| ATOM | 25 | P33 | UDP | 6 | 4.704 | −2.502 | −5.297 | −0.66 | +1.91 | +1.210 | 2.094 |
| ATOM | 26 | O36 | UDP | 6 | 6.116 | −2.693 | −4.873 | −0.44 | −2.01 | −0.850 | 2.094 |
| ATOM | 27 | O35 | UDP | 6 | 4.058 | −1.170 | −5.169 | −0.13 | −0.31 | −0.255 | 2.094 |
| ATOM | 28 | O34 | UDP | 6 | 4.493 | −2.893 | −6.851 | −0.20 | −1.73 | −0.850 | 2.094 |
| TER | | | | | | | | | | | |

ENDMDL

| | |
|---|---|
| MODEL | 42 |

| USER | Run = 42 | | | | | | | | | | |
|---|---|---|---|---|---|---|---|---|---|---|---|
| USER | Cluster Rank = 7 | | | | | | | | | | |
| USER | Number of conformations in this cluster = 7 | | | | | | | | | | |
| USER | | | | | | | | | | | |
| USER | RMSD from reference structure = 1.403 A | | | | | | | | | | |
| USER | | | | | | | | | | | |
| USER | Estimated Free Energy of Binding | | | | | = −9.01 kcal/mol [= (1) + (3)] | | | | | |
| USER | Estimated Inhibition Constant, Ki | | | | | = +2.48e−07 [Temperature = 298.15 K] | | | | | |
| USER | | | | | | | | | | | |
| USER | Final Docked Energy | | | | | = −11.81 kcal/mol [= (1) + (2)] | | | | | |
| USER | | | | | | | | | | | |
| USER | (1) Final Intermolecular Energy | | | | | = −11.50 kcal/mol | | | | | |
| USER | (2) Final Internal Energy of Ligand | | | | | = −0.30 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.002207 −8.061057 −5.538286 | | | | | | | | | | |
| USER | NEWDPF quat0 −0.361604 0.832154 −0.420432 8.389416 | | | | | | | | | | |
| USER | NEWDPF ndihe8 | | | | | | | | | | |
| USER | NEWDPF dihe0 7.07 72.36 21.51 15.45 7.25 −64.77 −81.28 150.62 | | | | | | | | | | |
| USER | | | | | | | | | | | |
| USER | | | | Rank | x | y | z | vdW | Elec | q | RMS |
| ATOM | 1 | C19 | UDP | 7 | −1.246 | −9.467 | −6.107 | −0.49 | +0.10 | +0.174 | 1.403 |
| ATOM | 2 | C1 | UDP | 7 | −2.093 | −8.317 | −5.562 | −0.38 | +0.07 | +0.113 | 1.403 |
| ATOM | 3 | C3 | UDP | 7 | −1.383 | −7.109 | −6.217 | −0.38 | +0.03 | +0.113 | 1.403 |
| ATOM | 4 | O16 | UDP | 7 | 0.079 | −8.964 | −5.984 | −0.04 | −0.12 | −0.227 | 1.403 |
| ATOM | 5 | C14 | UDP | 7 | 0.081 | −7.541 | −6.283 | −0.45 | +0.04 | +0.113 | 1.403 |
| ATOM | 6 | N10 | UDP | 7 | −1.297 | −10.696 | −5.306 | −0.33 | +0.03 | +0.039 | 1.403 |
| ATOM | 7 | C11 | UDP | 7 | −1.441 | −11.900 | −5.938 | −0.64 | −0.06 | −0.073 | 1.403 |
| ATOM | 8 | O17 | UDP | 7 | −1.528 | −11.984 | −7.165 | −0.22 | −0.02 | −0.026 | 1.403 |
| ATOM | 9 | N12 | UDP | 7 | −1.468 | −13.012 | −5.135 | −0.45 | −0.20 | −0.292 | 1.403 |
| ATOM | 10 | H12 | UDP | 7 | −1.566 | −13.977 | −5.632 | −0.27 | +0.14 | +0.393 | 1.403 |
| ATOM | 11 | C9 | UDP | 7 | −1.382 | −13.025 | −3.742 | −0.66 | −0.02 | −0.023 | 1.403 |
| ATOM | 12 | O18 | UDP | 7 | −1.415 | −14.115 | −3.169 | −0.11 | −0.02 | −0.026 | 1.403 |
| ATOM | 13 | C7 | UDP | 7 | −1.240 | −11.736 | −3.141 | −0.46 | +0.00 | +0.000 | 1.403 |
| ATOM | 14 | C5 | UDP | 7 | −1.218 | −10.642 | −3.918 | −0.20 | −0.03 | −0.050 | 1.403 |
| ATOM | 15 | O21 | UDP | 7 | −3.416 | −8.408 | −6.036 | −0.23 | −0.63 | −0.537 | 1.403 |
| ATOM | 16 | H21 | UDP | 7 | −3.988 | −8.933 | −5.359 | +0.08 | +0.45 | +0.424 | 1.403 |
| ATOM | 17 | O23 | UDP | 7 | −1.906 | −6.984 | −7.543 | −0.14 | −0.14 | −0.537 | 1.403 |
| ATOM | 18 | H23 | UDP | 7 | −2.216 | −6.016 | −7.700 | −0.17 | −0.09 | +0.424 | 1.403 |
| ATOM | 19 | C25 | UDP | 7 | 0.963 | −6.831 | −5.254 | −0.37 | +0.04 | +0.113 | 1.403 |
| ATOM | 20 | O28 | UDP | 7 | 2.187 | −6.506 | −5.885 | +0.03 | −0.20 | −0.368 | 1.403 |
| ATOM | 21 | P39 | UDP | 7 | 3.614 | −6.105 | −5.342 | −0.57 | +1.08 | +1.210 | 1.403 |
| ATOM | 22 | O30 | UDP | 7 | 3.747 | −6.658 | −3.965 | −0.09 | −0.77 | −0.850 | 1.403 |
| ATOM | 23 | O31 | UDP | 7 | 4.638 | −6.491 | −6.362 | −0.04 | −1.02 | −0.850 | 1.403 |
| ATOM | 24 | O32 | UDP | 7 | 3.317 | −4.541 | −5.270 | +0.05 | −0.49 | −0.510 | 1.403 |
| ATOM | 25 | P33 | UDP | 7 | 4.185 | −3.246 | −5.625 | −0.59 | +1.76 | +1.210 | 1.403 |
| ATOM | 26 | O36 | UDP | 7 | 5.441 | −3.536 | −4.885 | −0.25 | −1.66 | −0.850 | 1.403 |
| ATOM | 27 | O35 | UDP | 7 | 3.477 | −1.955 | −5.422 | −0.10 | −0.31 | −0.255 | 1.403 |
| ATOM | 28 | O34 | UDP | 7 | 4.407 | −3.324 | −7.224 | −0.25 | −1.73 | −0.850 | 1.403 |
| TER | | | | | | | | | | | |

ENDMDL

| | |
|---|---|
| MODEL | 16 |

| USER | Run = 16 | | | | | | | | | | |
|---|---|---|---|---|---|---|---|---|---|---|---|
| USER | Cluster Rank = 7 | | | | | | | | | | |
| USER | Number of conformations in this cluster = 7 | | | | | | | | | | |
| USER | | | | | | | | | | | |
| USER | RMSD from reference structure = 1.428 A | | | | | | | | | | |
| USER | | | | | | | | | | | |
| USER | Estimated Free Energy of Binding | | | | | = −9.21 kcal/mol [= (1) + (3)] | | | | | |
| USER | Estimated Inhibition Constant, Ki | | | | | = +1.77e−07 [Temperature = 298.15 K] | | | | | |
| USER | | | | | | | | | | | |
| USER | Final Docked Energy | | | | | = −11.64 kcal/mol [= (1) + (2)] | | | | | |
| USER | | | | | | | | | | | |
| USER | (1) Final Intermolecular Energy | | | | | = −11.70 kcal/mol | | | | | |
| USER | (2) Final Internal Energy of Ligand | | | | | = +0.06 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.774347 −8.091821 −5.085392 | | | | | | | | | | |
| USER | NEWDPF quat0 −0.326205 −0.760199 0.561861 −10.744813 | | | | | | | | | | |
| USER | NEWDPF ndihe8 | | | | | | | | | | |
| USER | NEWDPF dihe0 42.38 122.92 5.66 −35.93 15.79 −34.19 −46.64 −178.37 | | | | | | | | | | |
| USER | | | | | | | | | | | |
| USER | | | | Rank | x | y | z | vdW | Elec | q | RMS |
| ATOM | 1 | C19 | UDP | 7 | −1.099 | −9.411 | −5.802 | −0.47 | +0.10 | +0.174 | 1.428 |
| ATOM | 2 | C1 | UDP | 7 | −1.877 | −8.288 | −5.116 | −0.34 | +0.06 | +0.113 | 1.428 |
| ATOM | 3 | C3 | UDP | 7 | −1.114 | −7.052 | −5.647 | −0.36 | +0.03 | +0.113 | 1.428 |
| ATOM | 4 | O16 | UDP | 7 | 0.253 | −8.992 | −5.649 | +0.00 | −0.12 | −0.227 | 1.426 |
| ATOM | 5 | C14 | UDP | 7 | 0.325 | −7.547 | −5.788 | −0.42 | +0.04 | +0.113 | 1.428 |
| ATOM | 6 | N10 | UDP | 7 | −1.203 | −10.719 | −5.141 | −0.33 | +0.03 | +0.039 | 1.428 |
| ATOM | 7 | C11 | UDP | 7 | −1.241 | −11.852 | −5.905 | −0.67 | −0.06 | −0.073 | 1.428 |
| ATOM | 8 | O17 | UDP | 7 | −1.188 | −11.808 | −7.136 | −0.23 | −0.02 | −0.026 | 1.428 |
| ATOM | 9 | N12 | UDP | 7 | −1.324 | −13.042 | −5.227 | −0.47 | −0.20 | −0.292 | 1.428 |
| ATOM | 10 | H12 | UDP | 7 | −1.339 | −13.951 | −5.829 | −0.14 | +0.13 | +0.393 | 1.428 |
| ATOM | 11 | C9 | UDP | 7 | −1.394 | −13.199 | −3.843 | −0.67 | −0.02 | −0.023 | 1.428 |
| ATOM | 12 | O18 | UDP | 7 | −1.458 | −14.343 | −3.391 | −0.15 | −0.02 | −0.026 | 1.428 |
| ATOM | 13 | C7 | UDP | 7 | −1.356 | −11.978 | −3.101 | −0.52 | +0.00 | +0.000 | 1.428 |
| ATOM | 14 | C5 | UDP | 7 | −1.280 | −10.809 | −3.755 | −0.21 | −0.03 | −0.050 | 1.428 |
| ATOM | 15 | O21 | UDP | 7 | −3.209 | −8.259 | −5.572 | −0.21 | −0.55 | −0.537 | 1.428 |
| ATOM | 16 | H21 | UDP | 7 | −3.849 | −8.269 | −4.765 | +0.08 | +0.42 | +0.424 | 1.428 |
| ATOM | 17 | O23 | UDP | 7 | −1.648 | −6.754 | −6.941 | +0.04 | −0.03 | −0.537 | 1.428 |
| ATOM | 18 | H23 | UDP | 7 | −1.173 | −5.926 | −7.326 | −0.19 | −0.45 | +0.424 | 1.428 |
| ATOM | 19 | C25 | UDP | 7 | 1.255 | −7.002 | −4.703 | −0.38 | +0.05 | +0.113 | 1.428 |
| ATOM | 20 | O28 | UDP | 7 | 2.057 | −5.993 | −5.289 | +0.07 | −0.20 | −0.368 | 1.428 |
| ATOM | 21 | P39 | UDP | 7 | 3.606 | −5.691 | −5.272 | −0.53 | +1.13 | +1.210 | 1.428 |
| ATOM | 22 | O30 | UDP | 7 | 4.163 | −6.256 | −4.010 | −0.13 | −0.91 | −0.850 | 1.428 |
| ATOM | 23 | O31 | UDP | 7 | 4.194 | −6.143 | −6.570 | −0.06 | −0.95 | −0.850 | 1.428 |
| ATOM | 24 | O32 | UDP | 7 | 3.453 | −4.110 | −5.135 | +0.03 | −0.53 | −0.510 | 1.428 |
| ATOM | 25 | P33 | UDP | 7 | 4.404 | −2.884 | −5.518 | −0.64 | +1.84 | +1.210 | 1.428 |
| ATOM | 26 | O36 | UDP | 7 | 5.679 | −3.305 | −4.882 | −0.29 | −1.81 | −0.850 | 1.428 |
| ATOM | 27 | O35 | UDP | 7 | 3.828 | −1.545 | −5.227 | −0.15 | −0.31 | −0.255 | 1.428 |
| ATOM | 28 | O34 | UDP | 7 | 4.502 | −2.926 | −7.130 | −0.15 | −1.82 | −0.850 | 1.428 |
| TER | | | | | | | | | | | |

ENDMDL

| | |
|---|---|
| MODEL | 8 |

| USER | Run = 8 | | | | | | | | | | |
|---|---|---|---|---|---|---|---|---|---|---|---|
| USER | Cluster Rank = 7 | | | | | | | | | | |
| USER | Number of conformations in this cluster = 7 | | | | | | | | | | |
| USER | | | | | | | | | | | |
| USER | RMSD from reference structure = 1.526 A | | | | | | | | | | |
| USER | | | | | | | | | | | |
| USER | Estimated Free Energy of Binding | | | | | = −8.80 kcal/mol [= (1) + (3)] | | | | | |
| USER | Estimated Inhibition Constant, Ki | | | | | = +3.53e−07 [Temperature = 298.15 K] | | | | | |
| USER | | | | | | | | | | | |
| USER | Final Docked Energy | | | | | = −11.51 kcal/mol [= (1) + (2)] | | | | | |
| USER | | | | | | | | | | | |
| USER | (1) Final Intermolecular Energy | | | | | = −11.29 kcal/mol | | | | | |
| USER | (2) Final Internal Energy of Ligand | | | | | = −0.22 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.879901 −7.949029 −5.794197 | | | | | | | | | | |
| USER | NEWDPF quat0 −0.681332 −0.681711 −0.266563 9.390671 | | | | | | | | | | |
| USER | NEWDPF ndihe8 | | | | | | | | | | |
| USER | NEWDPF dihe0 9.34 94.26 −29.92 172.88 −2.08 −178.30 −109.42 27.47 | | | | | | | | | | |
| USER | | | | | | | | | | | |
| USER | | | | Rank | x | y | z | vdW | Elec | q | RMS |
| ATOM | 1 | C19 | UDP | 7 | −0.959 | −9.394 | −6.308 | −0.52 | +0.10 | +0.174 | 1.526 |
| ATOM | 2 | C1 | UDP | 7 | −1.931 | −8.244 | −6.048 | −0.42 | +0.06 | +0.113 | 1.526 |
| ATOM | 3 | C3 | UDP | 7 | −1.113 | −7.054 | −6.604 | −0.36 | +0.02 | +0.113 | 1.526 |
| ATOM | 4 | O16 | UDP | 7 | 0.293 | −8.839 | −5.920 | −0.04 | −0.12 | −0.227 | 1.526 |
| ATOM | 5 | C14 | UDP | 7 | 0.335 | −7.438 | −6.305 | −0.45 | +0.04 | +0.113 | 1.526 |
| ATOM | 6 | N10 | UDP | 7 | −1.168 | −10.574 | −5.458 | −0.33 | +0.03 | +0.039 | 1.526 |
| ATOM | 7 | C11 | UDP | 7 | −1.331 | −11.798 | −6.044 | −0.66 | −0.06 | −0.073 | 1.526 |
| ATOM | 8 | O17 | UDP | 7 | −1.306 | −11.942 | −7.268 | −0.21 | −0.02 | −0.026 | 1.526 |
| ATOM | 9 | N12 | UDP | 7 | −1.508 | −12.862 | −5.195 | −0.44 | −0.21 | −0.292 | 1.526 |
| ATOM | 10 | H12 | UDP | 7 | −1.623 | −13.845 | −5.654 | −0.26 | +0.13 | +0.393 | 1.526 |
| ATOM | 11 | C9 | UDP | 7 | −1.557 | −12.806 | −3.803 | −0.62 | −0.02 | −0.023 | 1.526 |
| ATOM | 12 | O18 | UDP | 7 | −1.717 | −13.859 | −3.184 | −0.09 | −0.02 | −0.026 | 1.526 |
| ATOM | 13 | C7 | UDP | 7 | −1.387 | −11.498 | −3.252 | −0.41 | +0.00 | +0.000 | 1.526 |
| ATOM | 14 | C5 | UDP | 7 | −1.218 | −10.451 | −4.073 | −0.22 | −0.03 | −0.050 | 1.526 |
| ATOM | 15 | O21 | UDP | 7 | −3.107 | −8.409 | −6.804 | −0.30 | −0.52 | −0.537 | 1.526 |
| ATOM | 16 | H21 | UDP | 7 | −3.822 | −8.877 | −6.229 | +0.07 | +0.47 | +0.424 | 1.526 |
| ATOM | 17 | O23 | UDP | 7 | −1.321 | −7.029 | −8.020 | −0.10 | −0.10 | −0.537 | 1.526 |
| ATOM | 18 | H23 | UDP | 7 | −1.242 | −6.061 | −8.361 | −0.25 | −0.12 | +0.424 | 1.526 |
| ATOM | 19 | C25 | UDP | 7 | 0.942 | −6.636 | −5.153 | −0.35 | +0.04 | +0.113 | 1.526 |
| ATOM | 20 | O28 | UDP | 7 | 2.340 | −6.859 | −5.161 | −0.01 | −0.21 | −0.368 | 1.526 |
| ATOM | 21 | P39 | UDP | 7 | 3.537 | −6.210 | −5.960 | −0.57 | +1.05 | +1.210 | 1.526 |
| ATOM | 22 | O30 | UDP | 7 | 4.786 | −6.918 | −5.558 | −0.20 | −0.94 | −0.850 | 1.526 |
| ATOM | 23 | O31 | UDP | 7 | 3.179 | −6.177 | −7.411 | −0.01 | −0.67 | −0.850 | 1.526 |
| ATOM | 24 | O32 | UDP | 7 | 3.459 | −4.795 | −5.231 | +0.03 | −0.50 | −0.510 | 1.526 |
| ATOM | 25 | P33 | UDP | 7 | 4.428 | −3.524 | −5.207 | −0.61 | +1.77 | +1.210 | 1.526 |
| ATOM | 26 | O36 | UDP | 7 | 5.729 | −4.167 | −4.882 | −0.16 | −1.78 | −0.850 | 1.526 |
| ATOM | 27 | O35 | UDP | 7 | 3.931 | −2.387 | −4.390 | −0.06 | −0.28 | −0.255 | 1.526 |
| ATOM | 28 | O34 | UDP | 7 | 4.407 | −2.960 | −6.721 | −0.24 | −1.61 | −0.850 | 1.526 |
| TER | | | | | | | | | | | |

ENDMDL

| | |
|---|---|
| MODEL | 48 |

| USER | Run = 48 | | | | | | | | | | |
|---|---|---|---|---|---|---|---|---|---|---|---|
| USER | Cluster Rank = 7 | | | | | | | | | | |
| USER | Number of conformations in this cluster = 7 | | | | | | | | | | |
| USER | | | | | | | | | | | |
| USER | RMSD from reference structure = 1.587 A | | | | | | | | | | |
| USER | | | | | | | | | | | |
| USER | Estimated Free Energy of Binding | | | | | = −8.43 kcal/mol [= (1) + (3)] | | | | | |
| USER | Estimated Inhibition Constant, Ki | | | | | = +6.61e−07 [Temperature = 298.15 K] | | | | | |
| USER | | | | | | | | | | | |
| USER | Final Docked Energy | | | | | = −11.17 kcal/mol [= (1) + (2)] | | | | | |
| USER | | | | | | | | | | | |
| USER | (1) Final Intermolecular Energy | | | | | = −10.92 kcal/mol | | | | | |
| USER | (2) Final Internal Energy of Ligand | | | | | = −0.25 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.771455 −8.035994 −5.687962 | | | | | | | | | | |
| USER | NEWDPF quat0 −0.620735 −0.582754 −0.524486 8.627764 | | | | | | | | | | |
| USER | NEWDPF ndihe8 | | | | | | | | | | |
| USER | NEWDPF dihe0 11.34 96.63 −93.54 167.70 1.33 −177.52 −106.00 123.25 | | | | | | | | | | |
| USER | | | | | | | | | | | |
| USER | | | | Rank | x | y | z | vdW | Elec | q | RMS |
| ATOM | 1 | C19 | UDP | 7 | −0.913 | −9.466 | −6.229 | −0.52 | +0.10 | +0.174 | 1.587 |
| ATOM | 2 | C1 | UDP | 7 | −1.837 | −8.288 | −5.925 | −0.41 | +0.06 | +0.113 | 1.587 |
| ATOM | 3 | C3 | UDP | 7 | −0.990 | −7.117 | −6.474 | −0.37 | +0.02 | +0.113 | 1.587 |
| ATOM | 4 | O16 | UDP | 7 | 0.366 | −8.964 | −5.857 | −0.03 | −0.12 | −0.227 | 1.587 |
| ATOM | 5 | C14 | UDP | 7 | 0.450 | −7.558 | −6.215 | −0.46 | +0.04 | +0.113 | 1.587 |
| ATOM | 6 | N10 | UDP | 7 | −1.146 | −10.656 | −5.399 | −0.34 | +0.03 | +0.039 | 1.587 |
| ATOM | 7 | C11 | UDP | 7 | −1.295 | −11.873 | −6.003 | −0.66 | −0.06 | −0.073 | 1.587 |
| ATOM | 8 | O17 | UDP | 7 | −1.238 | −12.002 | −7.228 | −0.15 | −0.02 | −0.026 | 1.587 |
| ATOM | 9 | N12 | UDP | 7 | −1.496 | −12.946 | −5.172 | −0.44 | −0.21 | −0.292 | 1.587 |
| ATOM | 10 | E12 | UDP | 7 | −1.599 | −13.923 | −5.645 | −0.25 | +0.13 | +0.393 | 1.587 |
| ATOM | 11 | C9 | UDP | 7 | −1.581 | −12.906 | −3.781 | −0.62 | −0.02 | −0.023 | 1.587 |
| ATOM | 12 | O18 | UDP | 7 | −1.758 | −13.966 | −3.179 | −0.09 | −0.02 | −0.026 | 1.587 |
| ATOM | 13 | C7 | UDP | 7 | −1.424 | −11.605 | −3.210 | −0.45 | +0.00 | +0.000 | 1.587 |
| ATOM | 14 | C5 | UDP | 7 | −1.232 | −10.549 | −4.014 | −0.22 | −0.03 | −0.050 | 1.587 |
| ATOM | 15 | O21 | UDP | 7 | −3.035 | −8.396 | −6.658 | −0.29 | −0.51 | −0.537 | 1.587 |
| ATOM | 16 | H21 | UDP | 7 | −3.763 | −8.820 | −6.065 | +0.07 | +0.47 | +0.424 | 1.587 |
| ATOM | 17 | O23 | UDP | 7 | −1.226 | −7.054 | −7.885 | −0.10 | −0.09 | −0.537 | 1.587 |
| ATOM | 18 | H23 | UDP | 7 | −1.081 | −6.091 | −8.215 | −0.25 | −0.15 | +0.424 | 1.587 |
| ATOM | 19 | C25 | UDP | 7 | 1.109 | −6.802 | −5.060 | −0.37 | +0.04 | +0.113 | 1.587 |
| ATOM | 20 | O28 | UDP | 7 | 2.507 | −7.009 | −5.151 | −0.04 | −0.21 | −0.368 | 1.587 |
| ATOM | 21 | P39 | UDP | 7 | 3.657 | −6.302 | −5.968 | −0.59 | +1.07 | +1.210 | 1.587 |
| ATOM | 22 | O30 | UDP | 7 | 4.948 | −6.936 | −5.575 | −0.19 | −0.98 | −0.850 | 1.587 |
| ATOM | 23 | O31 | UDP | 7 | 3.282 | −6.301 | −7.416 | +0.02 | −0.68 | −0.850 | 1.587 |
| ATOM | 24 | O32 | UDP | 7 | 3.509 | −4.887 | −5.250 | +0.03 | −0.51 | −0.510 | 1.587 |
| ATOM | 25 | P33 | UDP | 7 | 4.383 | −4.123 | −4.151 | −0.39 | +1.50 | +1.210 | 1.587 |
| ATOM | 26 | O36 | UDP | 7 | 4.523 | −5.181 | −3.116 | −0.22 | −1.19 | −0.850 | 1.587 |
| ATOM | 27 | O35 | UDP | 7 | 3.869 | −2.778 | −3.782 | +0.11 | −0.26 | −0.255 | 1.587 |
| ATOM | 28 | O34 | UDP | 7 | 5.791 | −3.818 | −4.882 | −0.21 | −1.87 | −0.850 | 1.587 |
| TER | | | | | | | | | | | |

ENDMDL

| | |
|---|---|
| MODEL | 1 |

| USER | Run = 1 | |
|---|---|---|
| USER | Cluster Rank = 7 | |
| USER | Number of conformations in this cluster = 7 | |
| USER | | |
| USER | RMSD from reference structure | = 1.273 A |
| USER | | |
| USER | Estimated Free Energy of Binding | = −8.12 kcal/mol [= (1) + (3)] |
| USER | Estimated Inhibition Constant, Ki | = +1.11e−06 [Temperature = 298.15 K] |
| USER | | |
| USER | Final Docked Energy | = −10.79 kcal/mol [= (1) + (2)] |
| USER | | |
| USER | (1) Final Intermolecular Energy | = −10.61 kcal/mol |
| USER | (2) Final Internal Energy of Ligand | = −0.18 kcal/mol |
| USER | (3) Torsional Free Energy | = +2.49 kcal/mol |
| USER | | |
| USER | | |

| USER | DPF = ligand_3.dpf | | | | | | | | | | |
|---|---|---|---|---|---|---|---|---|---|---|---|
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.036091 −8.065637 −5.469619 | | | | | | | | | | |
| USER | NEWDPF quat0 −0.315820 −0.947573 0.048616 −2.804966 | | | | | | | | | | |
| USER | NEWDPF ndihe8 | | | | | | | | | | |
| USER | NEWDPF dihe0 47.56 72.38 −18.67 45.91 7.30 −119.48 −102.10 −16.59 | | | | | | | | | | |
| USER | | | | | | | | | | | |
| USER | | | | Rank | x | y | z | vdW | Elec | q | RMS |
| ATOM | 1 | C19 | UDP | 7 | −1.154 | −9.438 | −6.149 | −0.49 | +0.10 | +0.174 | 1.273 |
| ATOM | 2 | C1 | UDP | 7 | −2.105 | −8.378 | −5.595 | −0.38 | +0.07 | +0.113 | 1.273 |
| ATOM | 3 | C3 | UDP | 7 | −1.421 | −7.090 | −6.110 | −0.38 | +0.03 | +0.113 | 1.273 |
| ATOM | 4 | O16 | UDP | 7 | 0.127 | −8.874 | −5.890 | −0.03 | −0.12 | −0.227 | 1.273 |
| ATOM | 5 | C14 | UDP | 7 | 0.067 | −7.436 | −6.091 | −0.42 | +0.03 | +0.113 | 1.273 |
| ATOM | 6 | N10 | UDP | 7 | −1.190 | −10.723 | −5.439 | −0.34 | +0.03 | +0.039 | 1.273 |
| ATOM | 7 | C11 | UDP | 7 | −1.214 | −11.884 | −6.160 | −0.63 | −0.06 | −0.073 | 1.273 |
| ATOM | 8 | O17 | UDP | 7 | −1.204 | −11.884 | −7.393 | −0.21 | −0.02 | −0.026 | 1.273 |
| ATOM | 9 | N12 | UDP | 7 | −1.234 | −13.051 | −5.437 | −0.40 | −0.20 | −0.292 | 1.273 |
| ATOM | 10 | H12 | UDP | 7 | −1.237 | −13.982 | −6.005 | −0.13 | +0.11 | +0.393 | 1.273 |
| ATOM | 11 | C9 | UDP | 7 | −1.252 | −13.159 | −4.047 | −0.71 | −0.02 | −0.023 | 1.273 |
| ATOM | 12 | O18 | UDP | 7 | −1.261 | −14.287 | −3.552 | −0.20 | −0.02 | −0.026 | 1.273 |
| ATOM | 13 | C7 | UDP | 7 | −1.232 | −11.911 | −3.351 | −0.52 | +0.00 | +0.000 | 1.273 |
| ATOM | 14 | C5 | UDP | 7 | −1.218 | −10.764 | −4.048 | −0.25 | −0.03 | −0.050 | 1.273 |
| ATOM | 15 | O21 | UDP | 7 | −3.381 | −8.506 | −6.176 | −0.24 | −0.59 | −0.537 | 1.273 |
| ATOM | 16 | H21 | UDP | 7 | −4.100 | −8.465 | −5.439 | +0.07 | +0.55 | +0.424 | 1.273 |
| ATOM | 17 | O23 | UDP | 7 | −1.852 | −6.899 | −7.462 | −0.10 | −0.10 | −0.537 | 1.273 |
| ATOM | 18 | H23 | UDP | 7 | −2.206 | −5.940 | −7.577 | −0.18 | −0.14 | +0.424 | 1.273 |
| ATOM | 19 | C25 | UDP | 7 | 0.826 | −6.755 | −4.951 | −0.35 | +0.04 | +0.113 | 1.273 |
| ATOM | 20 | O28 | UDP | 7 | 2.201 | −6.755 | −5.287 | +0.01 | −0.20 | −0.368 | 1.273 |
| ATOM | 21 | P39 | UDP | 7 | 3.369 | −5.714 | −5.078 | −0.49 | +1.04 | +1.210 | 1.273 |
| ATOM | 22 | O30 | UDP | 7 | 3.437 | −5.395 | −3.623 | −0.01 | −0.76 | −0.850 | 1.273 |
| ATOM | 23 | O31 | UDP | 7 | 4.600 | −6.238 | −5.746 | −0.09 | −1.03 | −0.850 | 1.273 |
| ATOM | 24 | O32 | UDP | 7 | 2.654 | −4.523 | −5.860 | +0.07 | −0.41 | −0.510 | 1.273 |
| ATOM | 25 | P33 | UDP | 7 | 3.083 | −3.018 | −6.186 | −0.54 | +1.36 | +1.210 | 1.273 |
| ATOM | 26 | O36 | UDP | 7 | 3.487 | −2.540 | −4.838 | −0.03 | −0.94 | −0.850 | 1.273 |
| ATOM | 27 | O35 | UDP | 7 | 2.090 | −2.254 | −6.984 | −0.13 | −0.29 | −0.255 | 1.273 |
| ATOM | 28 | O34 | UDP | 7 | 4.356 | −3.156 | −7.173 | −0.26 | −1.68 | −0.850 | 1.273 |
| TER | | | | | | | | | | | |

ENDMDL

| | |
|---|---|
| MODEL | 44 |

| USER | Run = 44 | | | | | | | | | | |
|---|---|---|---|---|---|---|---|---|---|---|---|
| USER | Cluster Rank = 7 | | | | | | | | | | |
| USER | Number of conformations in this cluster = 7 | | | | | | | | | | |
| USER | | | | | | | | | | | |
| USER | RMSD from reference structure | | | | | = 1.201 A | | | | | |
| USER | | | | | | | | | | | |
| USER | Estimated Free Energy of Binding | | | | | = −8.06 kcal/mol [= (1) + (3)] | | | | | |
| USER | Estimated Inhibition Constant, Ki | | | | | = +1.24e−06 [Temperature = 298.15 K] | | | | | |
| USER | | | | | | | | | | | |
| USER | Final Docked Energy | | | | | = −10.70 kcal/mol [= (1) + (2)] | | | | | |
| USER | | | | | | | | | | | |
| USER | (1) Final Intermolecular Energy | | | | | = −10.55 kcal/mol | | | | | |
| USER | (2) Final Internal Energy of Ligand | | | | | = −0.15 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.098799 −8.237813 −5.185093 | | | | | | | | | | |
| USER | NEWDPF quat0 0.049048 −0.696987 0.715404 −4.227680 | | | | | | | | | | |
| USER | NEWDPF ndihe8 | | | | | | | | | | |
| USER | NEWDPF dihe0 37.20 107.81 −61.20 −75.12 4.82 27.50 −35.16 118.81 | | | | | | | | | | |
| USER | | | | | | | | | | | |
| USER | | | | Rank | x | y | z | vdW | Elec | q | RMS |
| ATOM | 1 | C19 | UDP | 7 | −1.289 | −9.616 | −5.836 | −0.48 | +0.11 | +0.174 | 1.201 |
| ATOM | 2 | C1 | UDP | 7 | −2.182 | −8.499 | −5.299 | −0.36 | +0.07 | +0.113 | 1.201 |
| ATOM | 3 | C3 | UDP | 7 | −1.439 | −7.257 | −5.844 | −0.39 | +0.04 | +0.113 | 1.201 |
| ATOM | 4 | O16 | UDP | 7 | 0.020 | −9.112 | −5.594 | +0.01 | −0.13 | −0.227 | 1.201 |
| ATOM | 5 | C14 | UDP | 7 | 0.030 | −7.677 | −5.824 | −0.42 | +0.04 | +0.113 | 1.201 |
| ATOM | 6 | N10 | UDP | 7 | −1.385 | −10.883 | −5.099 | −0.33 | +0.03 | +0.039 | 1.201 |
| ATOM | 7 | C11 | UDP | 7 | −1.523 | −12.051 | −5.796 | −0.61 | −0.06 | −0.073 | 1.201 |
| ATOM | 8 | O17 | UDP | 7 | −1.567 | −12.073 | −7.028 | −0.21 | −0.02 | −0.026 | 1.201 |
| ATOM | 9 | N12 | UDP | 7 | −1.593 | −13.202 | −5.050 | −0.40 | −0.20 | −0.292 | 1.201 |
| ATOM | 10 | H12 | UDP | 7 | −1.686 | −14.139 | −5.598 | −0.27 | +0.11 | +0.393 | 1.201 |
| ATOM | 11 | C9 | UDP | 7 | −1.558 | −13.285 | −3.658 | −0.64 | −0.02 | −0.023 | 1.201 |
| ATOM | 12 | O18 | UDP | 7 | −1.626 | −14.401 | −3.141 | −0.10 | −0.02 | −0.026 | 1.201 |
| ATOM | 13 | C7 | UDP | 7 | −1.419 | −12.029 | −2.989 | −0.52 | +0.00 | +0.000 | 1.201 |
| ATOM | 14 | C5 | UDP | 7 | −1.355 | −10.899 | −3.708 | −0.27 | −0.03 | −0.050 | 1.201 |
| ATOM | 15 | O21 | UDP | 7 | −3.467 | −8.575 | −5.870 | −0.22 | −0.58 | −0.537 | 1.201 |
| ATOM | 16 | H21 | UDP | 7 | −4.172 | −8.658 | −5.123 | +0.07 | +0.48 | +0.424 | 1.201 |
| ATOM | 17 | O23 | UDP | 7 | −1.867 | −7.072 | −7.197 | −0.15 | −0.16 | −0.537 | 1.201 |
| ATOM | 18 | H23 | UDP | 7 | −1.613 | −6.126 | −7.511 | −0.22 | −0.25 | +0.424 | 1.201 |
| ATOM | 19 | C25 | UDP | 7 | 0.829 | −7.011 | −4.702 | −0.36 | +0.04 | +0.113 | 1.201 |
| ATOM | 20 | O28 | UDP | 7 | 1.417 | −5.836 | −5.229 | +0.09 | −0.16 | −0.368 | 1.201 |
| ATOM | 21 | P39 | UDP | 7 | 2.582 | −5.576 | −6.262 | −0.43 | +0.81 | +1.210 | 1.201 |
| ATOM | 22 | O30 | UDP | 7 | 3.849 | −6.068 | −5.649 | −0.05 | −0.83 | −0.850 | 1.201 |
| ATOM | 23 | O31 | UDP | 7 | 2.176 | −6.132 | −7.589 | −0.03 | −0.47 | −0.850 | 1.201 |
| ATOM | 24 | O32 | UDP | 7 | 2.518 | −3.986 | −6.168 | +0.05 | −0.42 | −0.510 | 1.201 |
| ATOM | 25 | P33 | UDP | 7 | 3.640 | −2.850 | −6.087 | −0.59 | +1.57 | +1.210 | 1.201 |
| ATOM | 26 | O36 | UDP | 7 | 4.486 | −3.368 | −4.981 | −0.10 | −1.22 | −0.850 | 1.201 |
| ATOM | 27 | O35 | UDP | 7 | 3.109 | −1.463 | −6.038 | −0.17 | −0.34 | −0.255 | 1.201 |
| ATOM | 28 | O34 | UDP | 7 | 4.408 | −2.926 | −7.507 | −0.14 | −1.76 | −0.850 | 1.201 |
| TER | | | | | | | | | | | |

ENDMDL

| | |
|---|---|
| MODEL | 39 |

| USER | Run = 39 | | | | | | | | | | |
|---|---|---|---|---|---|---|---|---|---|---|---|
| USER | Cluster Rank = 7 | | | | | | | | | | |
| USER | Number of conformations in this cluster = 7 | | | | | | | | | | |
| USER | | | | | | | | | | | |
| USER | RMSD from reference structure | | | | | = 1.127 A | | | | | |
| USER | | | | | | | | | | | |
| USER | Estimated Free Energy of Binding | | | | | = −7.87 kcal/mol [= (1) + (3)] | | | | | |
| USER | Estimated Inhibition Constant, Ki | | | | | = +1.70e−06 [Temperature = 298.15 K] | | | | | |
| USER | | | | | | | | | | | |
| USER | Final Docked Energy | | | | | = −10.63 kcal/mol [= (1) + (2)] | | | | | |
| USER | | | | | | | | | | | |
| USER | (1) Final Intermolecular Energy | | | | | = −10.36 kcal/mol | | | | | |
| USER | (2) Final Internal Energy of Ligand | | | | | = −0.27 kcal/mol | | | | | |
| USER | (3) Torsional Free Energy | | | | | = +2.49 kca1/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.095795 −7.986866 −5.569457 | | | | | | | | | | |
| USER | NEWDPF quat0 0.789896 −0.527010 −0.313567 −2.537097 | | | | | | | | | | |
| USER | NEWDPF ndihe8 | | | | | | | | | | |
| USER | NEWDPF dihe0 6.09 100.37 −101.87 156.31 4.99 159.87 −92.67 −5.86 | | | | | | | | | | |
| USER | | | | | | | | | | | |
| USER | | | | Rank | x | y | z | vdW | Elec | q | RMS |
| ATOM | 1 | C19 | UDP | 7 | −1.174 | −9.393 | −6.182 | −0.49 | +0.10 | +0.174 | 1.127 |
| ATOM | 2 | C1 | UDP | 7 | −2.156 | −8.324 | −5.703 | −0.39 | +0.07 | +0.113 | 1.127 |
| ATOM | 3 | C3 | UDP | 7 | −1.482 | −7.051 | −6.267 | −0.38 | +0.03 | +0.113 | 1.127 |
| ATOM | 4 | O16 | UDP | 7 | 0.090 | −8.795 | −5.922 | −0.02 | −0.12 | −0.227 | 1.127 |
| ATOM | 5 | C14 | UDP | 7 | 0.011 | −7.369 | −6.196 | −0.42 | +0.03 | +0.113 | 1.127 |
| ATOM | 6 | N10 | UDP | 7 | −1.206 | −10.642 | −5.409 | −0.33 | +0.03 | +0.039 | 1.127 |
| ATOM | 7 | C11 | UDP | 7 | −1.242 | −11.837 | −6.071 | −0.65 | −0.06 | −0.073 | 1.127 |
| ATOM | 8 | O17 | UDP | 7 | −1.246 | −11.898 | −7.303 | −0.22 | −0.02 | −0.026 | 1.127 |
| ATOM | 9 | N12 | UDP | 7 | −1.256 | −12.967 | −5.292 | −0.47 | −0.21 | −0.292 | 1.127 |
| ATOM | 10 | H12 | UDP | 7 | −1.268 | −13.924 | −5.813 | −0.20 | +0.15 | +0.393 | 1.127 |
| ATOM | 11 | C9 | UDP | 7 | −1.258 | −13.006 | −3.898 | −0.69 | −0.02 | −0.023 | 1.127 |
| ATOM | 12 | O18 | UDP | 7 | −1.266 | −14.108 | −3.348 | −0.16 | −0.02 | −0.026 | 1.127 |
| ATOM | 13 | C7 | UDP | 7 | −1.227 | −11.725 | −3.265 | −0.45 | +0.00 | +0.000 | 1.127 |
| ATOM | 14 | C5 | UDP | 7 | −1.218 | −10.615 | −4.017 | −0.23 | −0.03 | −0.050 | 1.127 |
| ATOM | 15 | O21 | UDP | 7 | −3.416 | −8.504 | −6.305 | −0.25 | −0.60 | −0.537 | 1.127 |
| ATOM | 16 | H21 | UDP | 7 | −4.003 | −9.100 | −5.704 | +0.07 | +0.46 | +0.424 | 1.127 |
| ATOM | 17 | O23 | UDP | 7 | −1.884 | −6.935 | −7.636 | −0.13 | −0.12 | −0.537 | 1.127 |
| ATOM | 18 | H23 | UDP | 7 | −1.802 | −5.955 | −7.936 | −0.23 | −0.19 | +0.424 | 1.127 |
| ATOM | 19 | C25 | UDP | 7 | 0.731 | −6.619 | −5.075 | −0.34 | +0.04 | +0.113 | 1.127 |
| ATOM | 20 | O28 | UDP | 7 | 2.072 | −6.419 | −5.482 | +0.04 | −0.19 | −0.368 | 1.127 |
| ATOM | 21 | P39 | UDP | 7 | 2.738 | −5.637 | −6.681 | −0.47 | +0.85 | +1.210 | 1.127 |
| ATOM | 22 | O30 | UDP | 7 | 4.191 | −5.497 | −6.379 | −0.01 | −1.07 | −0.850 | 1.127 |
| ATOM | 23 | O31 | UDP | 7 | 2.341 | −6.301 | −7.961 | −0.04 | −0.52 | −0.850 | 1.127 |
| ATOM | 24 | O32 | UDP | 7 | 1.991 | −4.258 | −6.398 | +0.04 | −0.32 | −0.510 | 1.127 |
| ATOM | 25 | P33 | UDP | 7 | 2.413 | −2.927 | −5.618 | −0.49 | +1.26 | +1.210 | 1.127 |
| ATOM | 26 | O36 | UDP | 7 | 2.890 | −3.494 | −4.330 | +0.06 | −0.79 | −0.850 | 1.127 |
| ATOM | 27 | O35 | UDP | 7 | 1.383 | −1.856 | −5.629 | −0.56 | −0.43 | −0.255 | 1.127 |
| ATOM | 28 | O34 | UDP | 7 | 3.631 | −2.310 | −6.482 | −0.11 | −1.15 | −0.850 | 1.127 |
| TER | | | | | | | | | | | |

ENDMDL

| | |
|---|---|
| MODEL | 34 |

| USER | Run = 34 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 1.401 A | | | | | |
| USER | | | | | | | | | | | |
| USER | Estimated Free Energy of Binding | | | | | = −8.67 kcal/mol [= (1) + (3)] | | | | | |
| USER | Estimated Inhibition Constant, Ki | | | | | = +4.38e−07 [Temperature = 298.15 K] | | | | | |
| USER | | | | | | | | | | | |
| USER | Final Docked Energy | | | | | = −11.51 kcal/mol [= (1) + (2)] | | | | | |
| USER | | | | | | | | | | | |
| USER | (1) Final Intermolecular Energy | | | | | = −11.17 kcal/mol | | | | | |
| USER | (2) Final Internal Energy of Ligand | | | | | = −0.34 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.201586 −7.930685 −5.845214 | | | | | | | | | | |
| USER | NEWDPF quat0 −0.716298 −0.472628 0.513361 6.920271 | | | | | | | | | | |
| USER | NEWDPF ndihe8 | | | | | | | | | | |
| USER | NEWDPF dihe0 16.44 95.45 −107.70 161.32 −0.03 −92.36 −44.17 62.17 | | | | | | | | | | |
| USER | | | | | | | | | | | |
| USER | | | | Rank | x | y | z | vdW | Elec | q | RMS |
| ATOM | 1 | C19 | UDP | 8 | −1.163 | −9.367 | −6.387 | −0.51 | +0.10 | +0.174 | 1.401 |
| ATOM | 2 | C1 | UDP | 8 | −2.230 | −8.329 | −6.044 | −0.43 | +0.07 | +0.113 | 1.401 |
| ATOM | 3 | C3 | UDP | 8 | −1.575 | −7.051 | −6.619 | −0.39 | +0.02 | +0.113 | 1.401 |
| ATOM | 4 | O16 | UDP | 8 | 0.046 | −8.692 | −6.061 | −0.02 | −0.11 | −0.227 | 1.401 |
| ATOM | 5 | C14 | UDP | 8 | −0.079 | −7.288 | −6.415 | −0.43 | +0.03 | −0.113 | 1.401 |
| ATOM | 6 | N10 | UDP | 8 | −1.197 | −10.577 | −5.555 | −0.34 | +0.03 | +0.039 | 1.401 |
| ATOM | 7 | C11 | UDP | 8 | −1.226 | −11.803 | −6.159 | −0.64 | −0.06 | −0.073 | 1.401 |
| ATOM | 8 | O17 | UDP | 8 | −1.222 | −11.923 | −7.386 | −0.21 | −0.02 | −0.026 | 1.401 |
| ATOM | 9 | N12 | UDP | 8 | −1.243 | −12.893 | −5.326 | −0.47 | −0.21 | −0.292 | 1.401 |
| ATOM | 10 | H12 | UDP | 8 | −1.250 | −13.875 | −5.800 | −0.22 | +0.17 | +0.393 | 1.401 |
| ATOM | 11 | C9 | UDP | 8 | −1.254 | −12.865 | −3.932 | −0.68 | −0.02 | −0.023 | 1.401 |
| ATOM | 12 | O18 | UDP | 8 | −1.263 | −13.940 | −3.329 | −0.16 | −0.02 | −0.026 | 1.401 |
| ATOM | 13 | C7 | UDP | 8 | −1.230 | −11.555 | −3.361 | −0.40 | +0.00 | +0.000 | 1.401 |
| ATOM | 14 | C5 | UDP | 8 | −1.218 | −10.482 | −4.167 | −0.26 | −0.03 | −0.050 | 1.401 |
| ATOM | 15 | O21 | UDP | 8 | −3.427 | −8.603 | −6.733 | −0.26 | −0.56 | −0.537 | 1.401 |
| ATOM | 16 | H21 | UDP | 8 | −4.099 | −9.047 | −6.091 | +0.07 | +0.47 | +0.424 | 1.401 |
| ATOM | 17 | O23 | UDP | 8 | −1.869 | −7.023 | −8.019 | −0.16 | −0.15 | −0.537 | 1.401 |
| ATOM | 18 | H23 | UDP | 8 | −1.891 | −6.048 | −8.346 | −0.17 | −0.03 | +0.424 | 1.401 |
| ATOM | 19 | C25 | UDP | 8 | 0.511 | −6.447 | −5.281 | −0.32 | +0.03 | +0.113 | 1.401 |
| ATOM | 20 | O28 | UDP | 8 | 1.202 | −5.358 | −5.866 | +0.08 | −0.11 | −0.368 | 1.401 |
| ATOM | 21 | P39 | UDP | 8 | 1.842 | −4.039 | −5.280 | −0.33 | +0.92 | +1.210 | 1.401 |
| ATOM | 22 | O30 | UDP | 8 | 2.633 | −3.397 | −6.369 | +0.01 | −0.81 | −0.850 | 1.401 |
| ATOM | 23 | O31 | UDP | 8 | 0.764 | −3.239 | −4.622 | −0.26 | −1.48 | −0.850 | 1.401 |
| ATOM | 24 | O32 | UDP | 8 | 2.822 | −4.782 | −4.266 | +0.09 | −0.41 | −0.510 | 1.401 |
| ATOM | 25 | P33 | UDP | 8 | 4.327 | −5.306 | −4.387 | −0.62 | +1.43 | +1.210 | 1.401 |
| ATOM | 26 | O36 | UDP | 8 | 4.253 | −6.087 | −5.650 | −0.08 | −0.94 | −0.850 | 1.401 |
| ATOM | 27 | O35 | UDP | 8 | 4.863 | −5.932 | −3.151 | −0.37 | −0.40 | −0.255 | 1.401 |
| ATOM | 28 | O34 | UDP | 8 | 5.217 | −3.970 | −4.568 | −0.20 | −1.40 | −0.850 | 1.401 |
| TER | | | | | | | | | | | |

ENDMDL

| | |
|---|---|
| MODEL | 22 |

| USER | Run = 22 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 1.481 A | | | | | |
| USER | | | | | | | | | | | |
| USER | Estimated Free Energy of Binding | | | | | = −8.66 kcal/mol [= (1) + (3)] | | | | | |
| USER | Estimated Inhibition Constant, Ki | | | | | = +4.46e−07 [Temperature = 298.15 K] | | | | | |
| USER | | | | | | | | | | | |
| USER | Final Docked Energy | | | | | = −11.42 kcal/mol [= (1) + (2) ] | | | | | |
| USER | | | | | | | | | | | |
| USER | (1) Final Intermolecular Energy | | | | | = −11.15 kcal/mol | | | | | |
| USER | (2) Final Internal Energy of Ligand | | | | | = −0.27 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.176748 −7.860549 −5.992729 | | | | | | | | | | |
| USER | NEWDPF quat0 −0.594025 −0.717009 0.364735 7.634541 | | | | | | | | | | |
| USER | NEWDPF ndihe8 | | | | | | | | | | |
| USER | NEWDPF dihe0 40.60 105.50 −113.84 164.16 −2.25 −95.39 −45.52 −178.52 | | | | | | | | | | |
| USER | | | | | | | | | | | |
| USER | | | | Rank | x | y | z | vdW | Elec | q | RMS |
| ATOM | 1 | C19 | UDP | 8 | −1.133 | −9.293 | −6.546 | −0.52 | +0.10 | +0.174 | 1.481 |
| ATOM | 2 | C1 | UDP | 8 | −2.201 | −8.245 | −6.235 | −0.45 | +0.07 | +0.113 | 1.481 |
| ATOM | 3 | C3 | UDP | 8 | −1.509 | −6.970 | −6.772 | −0.21 | +0.02 | +0.113 | 1.481 |
| ATOM | 4 | O16 | UDP | 8 | 0.070 | −8.634 | −6.167 | −0.02 | −0.11 | −0.227 | 1.481 |
| ATOM | 5 | C14 | UDP | 8 | −0.026 | −7.226 | −6.513 | −0.43 | +0.03 | +0.113 | 1.481 |
| ATOM | 6 | N10 | UDP | 8 | −1.213 | −10.509 | −5.726 | −0.34 | +0.03 | +0.039 | 1.481 |
| ATOM | 7 | C11 | UDP | 8 | −1.279 | −11.727 | −6.344 | −0.64 | −0.06 | −0.073 | 1.481 |
| ATOM | 8 | O17 | UDP | 8 | −1.271 | −11.835 | −7.572 | −0.23 | −0.02 | −0.026 | 1.481 |
| ATOM | 9 | N12 | UDP | 8 | −1.338 | −12.825 | −5.523 | −0.40 | −0.21 | −0.292 | 1.481 |
| ATOM | 10 | H12 | UDP | 8 | −1.374 | −13.801 | −6.007 | −0.24 | +0.09 | +0.393 | 1.481 |
| ATOM | 11 | C9 | UDP | 8 | −1.358 | −12.811 | −4.128 | −0.67 | −0.02 | −0.023 | 1.481 |
| ATOM | 12 | O18 | UDP | 8 | −1.406 | −13.891 | −3.537 | −0.17 | −0.02 | −0.026 | 1.481 |
| ATOM | 13 | C7 | UDP | 8 | −1.294 | −11.508 | −3.544 | −0.41 | +0.00 | +0.000 | 1.481 |
| ATOM | 14 | C5 | UDP | 8 | −1.241 | −10.428 | −4.337 | −0.31 | −0.03 | −0.050 | 1.481 |
| ATOM | 15 | O21 | UDP | 8 | −3.374 | −8.499 | −6.972 | −0.27 | −0.54 | −0.537 | 1.481 |
| ATOM | 16 | H21 | UDP | 8 | −4.179 | −8.549 | −6.331 | +0.07 | +0.59 | +0.424 | 1.481 |
| ATOM | 17 | O23 | UDP | 8 | −1.749 | −6.926 | −8.183 | −0.14 | −0.13 | −0.537 | 1.481 |
| ATOM | 18 | H23 | UDP | 8 | −1.582 | −5.972 | −8.530 | −0.25 | −0.07 | −0.424 | 1.481 |
| ATOM | 19 | C25 | UDP | 8 | 0.530 | −6.402 | −5.350 | −0.32 | +0.03 | +0.113 | 1.481 |
| ATOM | 20 | O28 | UDP | 8 | 1.281 | −5.334 | −5.897 | +0.07 | −0.12 | −0.368 | 1.481 |
| ATOM | 21 | P39 | UDP | 8 | 1.877 | −4.004 | −5.290 | −0.34 | +0.94 | +1.210 | 1.481 |
| ATOM | 22 | O30 | UDP | 8 | 2.714 | −3.362 | −6.343 | +0.00 | −0.84 | −0.850 | 1.481 |
| ATOM | 23 | O31 | UDP | 8 | 0.759 | −3.210 | −4.693 | −0.25 | −1.51 | −0.850 | 1.481 |
| ATOM | 24 | O32 | UDP | 8 | 2.812 | −4.728 | −4.222 | +0.09 | −0.41 | −0.510 | 1.481 |
| ATOM | 25 | P33 | UDP | 8 | 4.284 | −5.348 | −4.301 | −0.62 | +1.41 | +1.210 | 1.481 |
| ATOM | 26 | O36 | UDP | 8 | 4.162 | −6.212 | −5.504 | −0.09 | −0.89 | −0.850 | 1.481 |
| ATOM | 27 | O35 | UDP | 8 | 4.782 | −5.914 | −3.020 | −0.41 | −0.40 | −0.255 | 1.481 |
| ATOM | 28 | O34 | UDP | 8 | 5.251 | −4.083 | −4.574 | −0.20 | −1.41 | −0.850 | 1.481 |
| TER | | | | | | | | | | | |

ENDMDL

| | |
|---|---|
| MODEL | 26 |

| USER | Run = 26 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 1.429 A | | | | | |
| USER | | | | | | | | | | | |
| USER | Estimated Free Energy of Binding | | | | | = −8.88 kcal/mol [= (1) + (3)] | | | | | |
| USER | Estimated Inhibition Constant, Ki | | | | | = +3.08e−07 [Temperature = 298.15 K] | | | | | |
| USER | | | | | | | | | | | |
| USER | Final Docked Energy | | | | | = −11.39 kcal/mol [= (1) + (2)] | | | | | |
| USER | | | | | | | | | | | |
| USER | (1) Final Intermolecular Energy | | | | | = −11.37 kcal/mol | | | | | |
| USER | (2) Final Internal Energy of Ligand | | | | | = −0.02 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.341901 −7.904705 −5.264523 | | | | | | | | | | |
| USER | NEWDPF quat0 0.228582 0.972304 0.048742 13.048923 | | | | | | | | | | |
| USER | NEWDPF ndihe8 | | | | | | | | | | |
| USER | NEWDPF dihe0 55.67 109.12 −109.63 163.66 13.93 −112.13 −54.80 −148.08 | | | | | | | | | | |
| USER | | | | | | | | | | | |
| USER | | | | Rank | x | y | z | vdW | Elec | q | RMS |
| ATOM | 1 | C19 | UDP | 8 | −1.561 | −9.254 | −5.965 | −0.45 | +0.10 | +0.174 | 1.429 |
| ATOM | 2 | C1 | UDP | 8 | −2.413 | −8.230 | −5.214 | −0.35 | +0.07 | +0.113 | 1.429 |
| ATOM | 3 | C3 | UDP | 8 | −1.843 | −6.913 | −5.791 | −0.35 | +0.04 | +0.113 | 1.429 |
| ATOM | 4 | O16 | UDP | 8 | −0.261 | −8.678 | −5.911 | +0.01 | −0.11 | −0.227 | 1.429 |
| ATOM | 5 | C14 | UDP | 8 | −0.371 | −7.235 | −6.043 | −0.39 | +0.03 | +0.113 | 1.429 |
| ATOM | 6 | N10 | UDP | 8 | −1.460 | −10.564 | −5.309 | −0.31 | +0.03 | +0.039 | 1.429 |
| ATOM | 7 | C11 | UDP | 8 | −1.460 | −11.696 | −6.075 | −0.65 | −0.06 | −0.073 | 1.429 |
| ATOM | 8 | O17 | UDP | 8 | −1.541 | −11.650 | −7.304 | −0.25 | −0.02 | −0.026 | 1.429 |
| ATOM | 9 | N12 | UDP | 8 | −1.348 | −12.888 | −5.403 | −0.42 | −0.21 | −0.292 | 1.429 |
| ATOM | 10 | H12 | UDP | 8 | −1.331 | −13.795 | −6.007 | −0.24 | +0.10 | +0.393 | 1.429 |
| ATOM | 11 | C9 | UDP | 8 | −1.256 | −13.048 | −4.020 | −0.70 | −0.02 | −0.023 | 1.429 |
| ATOM | 12 | O18 | UDP | 8 | −1.155 | −14.192 | −3.574 | −0.21 | −0.02 | −0.026 | 1.429 |
| ATOM | 13 | C7 | UDP | 8 | −1.269 | −11.828 | −3.275 | −0.49 | +0.00 | +0.000 | 1.429 |
| ATOM | 14 | C5 | UDP | 8 | −1.382 | −10.659 | −3.923 | −0.29 | −0.03 | −0.050 | 1.429 |
| ATOM | 15 | O21 | UDP | 8 | −3.769 | −8.360 | −5.569 | −0.10 | −0.68 | −0.537 | 1.429 |
| ATOM | 16 | H21 | UDP | 8 | −4.358 | −8.221 | −4.735 | +0.07 | +0.49 | +0.424 | 1.429 |
| ATOM | 17 | O23 | UDP | 8 | −2.505 | −6.681 | −7.039 | −0.25 | −0.28 | −0.537 | 1.429 |
| ATOM | 18 | H23 | UDP | 8 | −2.343 | −5.711 | −7.338 | −0.12 | −0.24 | +0.424 | 1.429 |
| ATOM | 19 | C25 | UDP | 8 | 0.568 | −6.581 | −5.027 | −0.32 | +0.04 | +0.113 | 1.429 |
| ATOM | 20 | O28 | UDP | 8 | 1.336 | −5.609 | −5.712 | +0.08 | −0.13 | −0.368 | 1.429 |
| ATOM | 21 | P39 | UDP | 8 | 1.992 | −4.242 | −5.276 | −0.33 | +0.89 | +1.210 | 1.429 |
| ATOM | 22 | O30 | UDP | 8 | 2.827 | −3.757 | −6.412 | +0.02 | −0.81 | −0.850 | 1.429 |
| ATOM | 23 | O31 | UDP | 8 | 0.918 | −3.342 | −4.753 | −0.26 | −1.22 | −0.850 | 1.429 |
| ATOM | 24 | O32 | UDP | 8 | 2.927 | −4.872 | −4.149 | +0.08 | −0.41 | −0.510 | 1.429 |
| ATOM | 25 | P33 | UDP | 8 | 4.407 | −5.476 | −4.169 | −0.64 | +1.45 | +1.210 | 1.429 |
| ATOM | 26 | O36 | UDP | 8 | 4.309 | −6.427 | −5.307 | −0.12 | −0.90 | −0.850 | 1.429 |
| ATOM | 27 | O35 | UDP | 8 | 4.900 | −5.939 | −2.846 | −0.42 | −0.44 | −0.255 | 1.429 |
| ATOM | 28 | O34 | UDP | 8 | 5.360 | −4.220 | −4.525 | −0.21 | −1.43 | −0.850 | 1.429 |
| TER | | | | | | | | | | | |

ENDMDL

| | |
|---|---|
| MODEL | 10 |

| USER | Run = 10 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 1.493 A | | | | | |
| USER | | | | | | | | | | | |
| USER | Estimated Free Energy of Binding | | | | | = −8.50 kcal/mol [= (1) + (3)] | | | | | |
| USER | Estimated Inhibition Constant, Ki | | | | | = +5.93e−07 [Temperature = 298.15 K] | | | | | |
| USER | | | | | | | | | | | |
| USER | Final Docked Energy | | | | | = −11.15 kcal/mol [= (1) + (2)] | | | | | |
| USER | | | | | | | | | | | |
| USER | (1) Final Intermolecular Energy | | | | | = −10.99 kcal/mol | | | | | |
| USER | (2) Final Internal Energy of Ligand | | | | | = −0.16 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.356727 −7.865096 −6.072977 | | | | | | | | | | |
| USER | NEWDPF quat0 −0.540705 −0.771116 0.336182 10.301571 | | | | | | | | | | |
| USER | NEWDPF ndihe8 | | | | | | | | | | |
| USER | NEWDPF dihe0 51.17 115.56 −111.77 −173.57 −5.38 −103.90 −56.53 96.76 | | | | | | | | | | |
| USER | | | | | | | | | | | |
| USER | | | | Rank | x | y | z | vdW | Elec | q | RMS |
| ATOM | 1 | C19 | UDP | 8 | −1.272 | −9.306 | −6.598 | −0.52 | +0.10 | +0.174 | 1.493 |
| ATOM | 2 | C1 | UDP | 8 | −2.365 | −8.267 | −6.352 | −0.46 | +0.08 | +0.113 | 1.493 |
| ATOM | 3 | C3 | UDP | 8 | −1.666 | −6.993 | −6.882 | −0.28 | +0.02 | +0.113 | 1.493 |
| ATOM | 4 | O16 | UDP | 8 | −0.094 | −8.625 | −6.179 | −0.01 | −0.11 | −0.227 | 1.493 |
| ATOM | 5 | C14 | UDP | 8 | −0.192 | −7.224 | −6.554 | −0.43 | +0.02 | +0.113 | 1.493 |
| ATOM | 6 | N10 | UDP | 8 | −1.372 | −10.509 | −5.761 | −0.33 | +0.03 | +0.039 | 1.493 |
| ATOM | 7 | C11 | UDP | 8 | −1.461 | −11.734 | −6.361 | −0.65 | −0.06 | −0.073 | 1.493 |
| ATOM | 8 | O17 | UDP | 8 | −1.458 | −11.859 | −7.588 | −0.25 | −0.02 | −0.026 | 1.493 |
| ATOM | 9 | N12 | UDP | 8 | −1.538 | −12.819 | −5.524 | −0.36 | −0.20 | −0.292 | 1.493 |
| ATOM | 10 | H12 | UDP | 8 | −1.593 | −13.801 | −5.994 | −0.25 | +0.02 | +0.393 | 1.493 |
| ATOM | 11 | C9 | UDP | 8 | −1.555 | −12.785 | −4.130 | −0.64 | −0.02 | −0.023 | 1.493 |
| ATOM | 12 | O18 | UDP | 8 | −1.622 | −13.855 | −3.523 | −0.14 | −0.02 | −0.026 | 1.493 |
| ATOM | 13 | C7 | UDP | 8 | −1.466 | −11.475 | −3.564 | −0.44 | +0.00 | +0.000 | 1.493 |
| ATOM | 14 | C5 | UDP | 8 | −1.395 | −10.408 | −4.373 | −0.36 | −0.03 | −0.050 | 1.493 |
| ATOM | 15 | O21 | UDP | 8 | −3.501 | −8.549 | −7.134 | −0.27 | −0.53 | −0.537 | 1.493 |
| ATOM | 16 | H21 | UDP | 8 | −4.351 | −8.425 | −6.564 | +0.08 | +0.65 | +0.424 | 1.493 |
| ATOM | 17 | O23 | UDP | 8 | −1.846 | −6.976 | −8.302 | −0.15 | −0.15 | −0.537 | 1.493 |
| ATOM | 18 | H23 | UDP | 8 | −1.515 | −6.078 | −8.679 | −0.19 | −0.02 | +0.424 | 1.493 |
| ATOM | 19 | C25 | UDP | 8 | 0.302 | −6.373 | −5.383 | −0.30 | +0.02 | +0.113 | 1.493 |
| ATOM | 20 | O28 | UDP | 8 | 1.265 | −5.465 | −5.886 | +0.07 | −0.11 | −0.368 | 1.493 |
| ATOM | 21 | P39 | UDP | 8 | 1.824 | −4.082 | −5.370 | −0.33 | +0.89 | +1.210 | 1.493 |
| ATOM | 22 | O30 | UDP | 8 | 2.532 | −3.428 | −6.507 | +0.00 | −0.78 | −0.850 | 1.493 |
| ATOM | 23 | O31 | UDP | 8 | 0.712 | −3.336 | −4.706 | −0.27 | −1.37 | −0.850 | 1.493 |
| ATOM | 24 | O32 | UDP | 8 | 2.886 | −4.712 | −4.363 | +0.08 | −0.42 | −0.510 | 1.493 |
| ATOM | 25 | P33 | UDP | 8 | 4.396 | −5.208 | −4.536 | −0.62 | +1.47 | +1.210 | 1.493 |
| ATOM | 26 | O36 | UDP | 8 | 4.268 | −6.093 | −5.723 | −0.08 | −0.95 | −0.850 | 1.493 |
| ATOM | 27 | O35 | UDP | 8 | 5.025 | −5.714 | −3.288 | −0.27 | −0.42 | −0.255 | 1.493 |
| ATOM | 28 | O34 | UDP | 8 | 5.233 | −3.870 | −4.882 | −0.20 | −1.52 | −0.850 | 1.493 |
| TER | | | | | | | | | | | |

ENDMDL

| | |
|---|---|
| MODEL | 12 |

| USER | Run = 12 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 1.208 A | | | | | |
| USER | | | | | | | | | | | |
| USER | Estimated Free Energy of Binding | | | | | = −8.44 kcal/mol [= (1) + (3)] | | | | | |
| USER | Estimated Inhibition Constant, Ki | | | | | = +6.54e−07 [Temperature = 298.15 K] | | | | | |
| USER | | | | | | | | | | | |
| USER | Final Docked Energy | | | | | = −10.92 kcal/mol [= (1) + (2)] | | | | | |
| USER | | | | | | | | | | | |
| USER | (1) Final Intermolecular Energy | | | | | = −10.93 kcal/mol | | | | | |
| USER | (2) Final Internal Energy of Ligand | | | | | = +0.01 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.031082 −8.105578 −5.575735 | | | | | | | | | | |
| USER | NEWDPF quat0 0.448568 −0.893551 0.018796 −0.606664 | | | | | | | | | | |
| USER | NEWDPF ndihe8 | | | | | | | | | | |
| USER | NEWDPF dihe0 28.65 92.28 −83.24 65.58 5.29 −18.34 5.63 −118.93 | | | | | | | | | | |
| USER | | | | | | | | | | | |
| USER | | | | Rank | x | y | z | vdW | Elec | q | RMS |
| ATOM | 1 | C19 | UDP | 8 | −1.121 | −9.492 | −6.231 | −0.51 | +0.10 | +0.174 | 1.208 |
| ATOM | 2 | C1 | UDP | 8 | −2.094 | −8.423 | −5.734 | −0.40 | +0.07 | +0.113 | 1.208 |
| ATOM | 3 | C3 | UDP | 8 | −1.394 | −7.144 | −6.250 | −0.39 | +0.03 | +0.113 | 1.208 |
| ATOM | 4 | O16 | UDP | 8 | 0.148 | −8.920 | −5.936 | −0.03 | −0.12 | −0.227 | 1.208 |
| ATOM | 5 | C14 | UDP | 8 | 0.092 | −7.486 | −6.168 | −0.43 | +0.04 | +0.113 | 1.208 |
| ATOM | 6 | N10 | UDP | 8 | −1.181 | −10.762 | −5.497 | −0.35 | +0.03 | +0.039 | 1.208 |
| ATOM | 7 | C11 | UDP | 8 | −1.218 | −11.936 | −6.196 | −0.60 | −0.06 | −0.073 | 1.208 |
| ATOM | 8 | O17 | UDP | 8 | −1.200 | −11.960 | −7.428 | −0.15 | −0.02 | −0.026 | 1.208 |
| ATOM | 9 | N12 | UDP | 8 | −1.259 | −13.089 | −5.452 | −0.34 | −0.20 | −0.292 | 1.208 |
| ATOM | 10 | H12 | UDP | 8 | −1.273 | −14.030 | −6.001 | −0.04 | +0.09 | +0.393 | 1.208 |
| ATOM | 11 | C9 | UDP | 8 | −1.287 | −13.170 | −4.059 | −0.70 | −0.02 | −0.023 | 1.208 |
| ATOM | 12 | O18 | UDP | 8 | −1.317 | −14.288 | −3.543 | −0.19 | −0.02 | −0.026 | 1.208 |
| ATOM | 13 | C7 | UDP | 8 | −1.253 | −11.909 | −3.387 | −0.52 | +0.00 | +0.000 | 1.208 |
| ATOM | 14 | C5 | UDP | 8 | −1.218 | −10.777 | −4.106 | −0.27 | −0.03 | −0.050 | 1.208 |
| ATOM | 15 | O21 | UDP | 8 | −3.348 | −8.567 | −6.359 | −0.26 | −0.57 | −0.537 | 1.208 |
| ATOM | 16 | H21 | UDP | 8 | −4.054 | −8.829 | −5.656 | +0.07 | +0.49 | +0.424 | 1.208 |
| ATOM | 17 | O23 | UDP | 8 | −1.775 | −6.981 | −7.620 | −0.12 | −0.12 | −0.537 | 1.208 |
| ATOM | 18 | H23 | UDP | 8 | −1.806 | −5.979 | −7.851 | −0.23 | −0.20 | +0.424 | 1.208 |
| ATOM | 19 | C25 | UDP | 8 | 0.808 | −6.779 | −5.015 | −0.35 | +0.04 | +0.113 | 1.208 |
| ATOM | 20 | O28 | UDP | 8 | 0.526 | −5.396 | −5.115 | +0.09 | −0.09 | −0.368 | 1.208 |
| ATOM | 21 | P39 | UDP | 8 | 1.422 | −4.122 | −5.372 | −0.30 | +0.81 | +1.210 | 1.208 |
| ATOM | 22 | O30 | UDP | 8 | 1.790 | −4.111 | −6.817 | +0.02 | −0.45 | −0.850 | 1.208 |
| ATOM | 23 | O31 | UDP | 8 | 0.723 | −2.926 | −4.810 | −0.21 | −1.92 | −0.850 | 1.208 |
| ATOM | 24 | O32 | UDP | 8 | 2.660 | −4.627 | −4.504 | +0.09 | −0.40 | −0.510 | 1.208 |
| ATOM | 25 | P33 | UDP | 8 | 4.167 | −5.012 | −4.870 | −0.57 | +1.42 | +1.210 | 1.208 |
| ATOM | 26 | O36 | UDP | 8 | 3.952 | −5.952 | −6.002 | −0.05 | −0.88 | −0.850 | 1.208 |
| ATOM | 27 | O35 | UDP | 8 | 4.999 | −5.418 | −3.708 | −0.20 | −0.38 | −0.255 | 1.208 |
| ATOM | 28 | O34 | UDP | 8 | 4.837 | −3.628 | −5.368 | −0.10 | −1.50 | −0.850 | 1.208 |
| TER | | | | | | | | | | | |

ENDMDL

| | |
|---|---|
| MODEL | 14 |

| USER | Run = 14 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 0.987 A | | | | | |
| USER | | | | | | | | | | | |
| USER | Estimated Free Energy of Binding | | | | | = −7.93 kcal/mol [= (1) + (3)] | | | | | |
| USER | Estimated Inhibition Constant, Ki | | | | | = +1.53e−06 [Temperature = 298.15 K] | | | | | |
| USER | | | | | | | | | | | |
| USER | Final Docked Energy | | | | | = −10.80 kcal/mol [= (1) + (2)] | | | | | |
| USER | | | | | | | | | | | |
| USER | (1) Final Intermolecular Energy | | | | | = −10.42 kcal/mol | | | | | |
| USER | (2) Final Internal Energy of Ligand | | | | | = −0.38 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.127703 −7.969744 −5.657029 | | | | | | | | | | |
| USER | NEWDPF quat0 0.795322 0.194661 −0.574082 −3.139641 | | | | | | | | | | |
| USER | NEWDPF ndihe8 | | | | | | | | | | |
| USER | NEWDPF dihe0 2.81 82.31 39.31 172.53 3.32 −162.25 −97.49 37.38 | | | | | | | | | | |
| USER | | | | | | | | | | | |
| USER | | | | Rank | x | y | z | vdW | Elec | q | RMS |
| ATOM | 1 | C19 | UDP | 8 | −1.160 | −9.382 | −6.260 | −0.50 | +0.10 | +0.174 | 0.987 |
| ATOM | 2 | C1 | UDP | 8 | −2.176 | −8.327 | −5.824 | −0.40 | +0.07 | +0.113 | 0.987 |
| ATOM | 3 | C3 | UDP | 8 | −1.507 | −7.047 | −6.375 | −0.38 | +0.03 | +0.113 | 0.987 |
| ATOM | 4 | O16 | UDP | 8 | 0.084 | −8.758 | −5.962 | −0.02 | −0.11 | −0.227 | 0.987 |
| ATOM | 5 | C14 | UDP | 8 | −0.012 | −7.337 | −6.251 | −0.42 | +0.03 | +0.113 | 0.987 |
| ATOM | 6 | N10 | UDP | 8 | −1.195 | −10.625 | −5.477 | −0.34 | +0.03 | +0.039 | 0.987 |
| ATOM | 7 | C11 | UDP | 8 | −1.222 | −11.825 | −6.132 | −0.64 | −0.06 | −0.073 | 0.987 |
| ATOM | 8 | O17 | UDP | 8 | −1.215 | −11.895 | −7.363 | −0.21 | −0.02 | −0.026 | 0.987 |
| ATOM | 9 | H12 | UDP | 8 | −1.240 | −12.949 | −5.344 | −0.46 | −0.21 | −0.292 | 0.987 |
| ATOM | 10 | H12 | UDP | 8 | −1.245 | −13.910 | −5.858 | −0.20 | +0.15 | +0.393 | 0.987 |
| ATOM | 11 | C9 | UDP | 8 | −1.254 | −12.978 | −3.950 | −0.69 | −0.02 | −0.023 | 0.987 |
| ATOM | 12 | O18 | UDP | 8 | −1.264 | −14.076 | −3.392 | −0.17 | −0.02 | −0.026 | 0.987 |
| ATOM | 13 | C7 | UDP | 8 | −1.232 | −11.692 | −3.326 | −0.45 | +0.00 | +0.000 | 0.987 |
| ATOM | 14 | C5 | UDP | 8 | −1.220 | −10.587 | −4.087 | −0.25 | −0.03 | −0.050 | 0.987 |
| ATOM | 15 | O21 | UDP | 8 | −3.412 | −8.535 | −6.466 | −0.26 | −0.59 | −0.537 | 0.987 |
| ATOM | 16 | H21 | UDP | 8 | −3.987 | −9.181 | −5.906 | +0.07 | +0.45 | +0.424 | 0.987 |
| ATOM | 17 | O23 | UDP | 8 | −1.864 | −6.949 | −7.758 | −0.14 | −0.13 | −0.537 | 0.987 |
| ATOM | 18 | H23 | UDP | 8 | −2.085 | −5.971 | −7.987 | −0.16 | −0.09 | +0.424 | 0.987 |
| ATOM | 19 | C25 | UDP | 8 | 0.656 | −6.564 | −5.113 | −0.33 | +0.04 | +0.113 | 0.987 |
| ATOM | 20 | O28 | UDP | 8 | 2.032 | −6.441 | −5.424 | +0.04 | −0.19 | −0.368 | 0.987 |
| ATOM | 21 | P39 | UDP | 8 | 2.906 | −5.276 | −6.032 | −0.43 | +0.95 | +1.210 | 0.987 |
| ATOM | 22 | O30 | UDP | 8 | 4.304 | −5.777 | −6.158 | −0.04 | −1.04 | −0.850 | 0.987 |
| ATOM | 23 | O31 | UDP | 8 | 2.226 | −4.761 | −7.260 | +0.01 | −0.51 | −0.850 | 0.987 |
| ATOM | 24 | O32 | UDP | 8 | 2.787 | −4.317 | −4.765 | +0.08 | −0.44 | −0.510 | 0.987 |
| ATOM | 25 | P33 | UDP | 8 | 2.239 | −2.828 | −4.568 | −0.41 | +1.30 | +1.210 | 0.987 |
| ATOM | 26 | O36 | UDP | 8 | 2.967 | −2.115 | −5.651 | −0.08 | −1.01 | −0.850 | 0.987 |
| ATOM | 27 | O35 | UDP | 8 | 2.300 | −2.329 | −3.170 | +0.05 | −0.21 | −0.255 | 0.987 |
| ATOM | 28 | O34 | UDP | 8 | 0.657 | −2.926 | −4.882 | −0.21 | −1.98 | −0.850 | 0.987 |
| TER | | | | | | | | | | | |

ENDMDL

| | |
|---|---|
| MODEL | 36 |

| USER | Run = 36 | | | | | | | | | | |
|---|---|---|---|---|---|---|---|---|---|---|---|
| USER | Cluster Rank = 8 | | | | | | | | | | |
| USER | Number of conformations in this cluster = 7 | | | | | | | | | | |
| USER | | | | | | | | | | | |
| USER | RMSD from reference structure | | | | | = 0.984 A | | | | | |
| USER | | | | | | | | | | | |
| USER | Estimated Free Energy of Binding | | | | | = −7.06 kcal/mol [= (1) + (3)] | | | | | |
| USER | Estimated Inhibition Constant, Ki | | | | | = +6.63e−06 [Temperature = 298.15 K] | | | | | |
| USER | | | | | | | | | | | |
| USER | Final Docked Energy | | | | | = −9.81 kcal/mol [= (1) + (2)] | | | | | |
| USER | | | | | | | | | | | |
| USER | (1) Final Intermolecular Energy | | | | | = −9.56 kcal/mol | | | | | |
| USER | (2) Final Internal Energy of Ligand | | | | | = −0.26 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.365593 −7.922776 −5.247683 | | | | | | | | | | |
| USER | NEWDPF quat0 −0.250453 0.647135 −0.720063 −5.807668 | | | | | | | | | | |
| USER | NEWDPF ndihe8 | | | | | | | | | | |
| USER | NEWDPF dihe0 56.95 62.74 57.91 179.99 3.09 172.35 −106.34 −125.40 | | | | | | | | | | |
| USER | | | | | | | | | | | |
| USER | | | | Rank | x | y | z | vdW | Elec | q | RMS |
| ATOM | 1 | C19 | UDP | 8 | −1.303 | −9.289 | −5.946 | −0.46 | +0.10 | +0.174 | 0.984 |
| ATOM | 2 | C1 | UDP | 8 | −2.387 | −8.311 | −5.495 | −0.38 | +0.08 | +0.113 | 0.984 |
| ATOM | 3 | C3 | UDP | 8 | −1.749 | −6.968 | −5.921 | −0.36 | +0.03 | +0.113 | 0.984 |
| ATOM | 4 | O16 | UDP | 8 | −0.106 | −8.637 | −5.540 | +0.01 | −0.11 | −0.227 | 0.984 |
| ATOM | 5 | C14 | UDP | 8 | −0.251 | −7.204 | −5.736 | −0.37 | +0.03 | +0.113 | 0.984 |
| ATOM | 6 | N10 | UDP | 8 | −1.323 | −10.584 | −5.254 | −0.32 | +0.03 | +0.039 | 0.984 |
| ATOM | 7 | C11 | UDP | 8 | −1.267 | −11.735 | −5.989 | −0.67 | −0.06 | −0.073 | 0.984 |
| ATOM | 8 | O17 | UDP | 8 | −1.198 | −11.718 | −7.220 | −0.25 | −0.02 | −0.026 | 0.984 |
| ATOM | 9 | N12 | UDP | 8 | −1.275 | −12.912 | −5.283 | −0.47 | −0.21 | −0.292 | 0.984 |
| ATOM | 10 | H12 | UDP | 8 | −1.214 | −13.834 | −5.861 | −0.23 | +0.17 | +0.393 | 0.984 |
| ATOM | 11 | C9 | UDP | 8 | −1.355 | −13.040 | −3.896 | −0.67 | −0.02 | −0.023 | 0.984 |
| ATOM | 12 | O18 | UDP | 8 | −1.344 | −14.174 | −3.416 | −0.17 | −0.02 | −0.026 | 0.984 |
| ATOM | 13 | C7 | UDP | 8 | −1.418 | −11.802 | −3.185 | −0.49 | +0.00 | +0.000 | 0.984 |
| ATOM | 14 | C5 | UDP | 8 | −1.416 | −10.646 | −3.867 | −0.29 | −0.03 | −0.050 | 0.984 |
| ATOM | 15 | O21 | UDP | 8 | −3.578 | −8.523 | −6.217 | −0.20 | −0.64 | −0.537 | 0.984 |
| ATOM | 16 | H21 | UDP | 8 | −4.387 | −8.390 | −5.593 | +0.07 | +0.63 | +0.424 | 0.984 |
| ATOM | 17 | O23 | UDP | 8 | −2.039 | −6.788 | −7.311 | −0.12 | −0.12 | −0.537 | 0.984 |
| ATOM | 18 | H23 | UDP | 8 | −2.592 | −5.930 | −7.438 | −0.15 | +0.00 | +0.424 | 0.984 |
| ATOM | 19 | C25 | UDP | 8 | 0.323 | −6.487 | −4.513 | −0.29 | +0.04 | +0.113 | 0.984 |
| ATOM | 20 | O28 | UDP | 8 | 1.733 | −6.469 | −4.645 | +0.06 | −0.18 | −0.368 | 0.984 |
| ATOM | 21 | P39 | UDP | 8 | 2.727 | −5.640 | −5.548 | −0.41 | +0.86 | +1.210 | 0.984 |
| ATOM | 22 | O30 | UDP | 8 | 4.085 | −6.233 | −5.388 | −0.08 | −0.86 | −0.850 | 0.984 |
| ATOM | 23 | O31 | UDP | 8 | 2.154 | −5.548 | −6.927 | +0.02 | −0.46 | −0.850 | 0.984 |
| ATOM | 24 | O32 | UDP | 8 | 2.621 | −4.295 | −4.701 | +0.09 | −0.42 | −0.510 | 0.984 |
| ATOM | 25 | P33 | UDP | 8 | 1.850 | −2.914 | −4.935 | −0.41 | +1.42 | +1.210 | 0.984 |
| ATOM | 26 | O36 | UDP | 8 | 2.278 | −2.584 | −6.320 | −0.06 | −0.92 | −0.850 | 0.984 |
| ATOM | 27 | O35 | UDP | 8 | 2.018 | −1.922 | −3.843 | −0.07 | −0.29 | −0.255 | 0.984 |
| ATOM | 28 | O34 | UDP | 8 | 0.282 | −3.301 | −4.876 | −0.20 | −1.71 | −0.850 | 0.984 |
| TER | | | | | | | | | | | |

ENDMDL

| | |
|---|---|
| MODEL | 38 |

| USER | Run = 38 | | | | | | | | | | |
|---|---|---|---|---|---|---|---|---|---|---|---|
| USER | Cluster Rank = 9 | | | | | | | | | | |
| USER | Number of conformations in this cluster = 5 | | | | | | | | | | |
| USER | | | | | | | | | | | |
| USER | RMSD from reference structure | | | | | = 1.478 A | | | | | |
| USER | | | | | | | | | | | |
| USER | Estimated Free Energy of Binding | | | | | = −8.41 kcal/mol [= (1) + (3)] | | | | | |
| USER | Estimated Inhibition Constant, Ki | | | | | = +6.84e−07 [Temperature = 298.15 K] | | | | | |
| USER | | | | | | | | | | | |
| USER | Final Docked Energy | | | | | = −11.32 kcal/mol [= (1) + (2)] | | | | | |
| USER | | | | | | | | | | | |
| USER | (1) Final Intermolecular Energy | | | | | = −10.90 kcal/mol | | | | | |
| USER | (2) Final Internal Energy of Ligand | | | | | = −0.42 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.046979 −7.996171 −5.541821 | | | | | | | | | | |
| USER | NEWDPF quat0 0.894621 0.368239 −0.253089 −1.612643 | | | | | | | | | | |
| USER | NEWDPF ndihe8 | | | | | | | | | | |
| USER | NEWDPF dihe0 19.73 86.06 52.40 −10.32 4.12 −147.72 −71.97 145.18 | | | | | | | | | | |
| USER | | | | | | | | | | | |
| USER | | | | Rank | x | y | z | vdW | Elec | q | RMS |
| ATOM | 1 | C19 | UDP | 9 | −1.113 | −9.396 | −6.171 | −0.49 | +0.10 | +0.174 | 1.478 |
| ATOM | 2 | C1 | UDP | 9 | −2.104 | −8.325 | −5.715 | −0.39 | +0.07 | +0.113 | 1.478 |
| ATOM | 3 | C3 | UDP | 9 | −1.404 | −7.051 | −6.243 | −0.38 | +0.02 | +0.113 | 1.478 |
| ATOM | 4 | O16 | UDP | 9 | 0.145 | −8.808 | −5.862 | −0.02 | −0.12 | −0.227 | 1.478 |
| ATOM | 5 | C14 | UDP | 9 | 0.084 | −7.380 | −6.125 | −0.42 | +0.03 | +0.113 | 1.478 |
| ATOM | 6 | N10 | UDP | 9 | −1.179 | −10.652 | −5.411 | −0.34 | +0.03 | +0.039 | 1.478 |
| ATOM | 7 | C11 | UDP | 9 | −1.218 | −11.839 | −6.087 | −0.65 | −0.06 | −0.073 | 1.478 |
| ATOM | 8 | O17 | UDP | 9 | −1.197 | −11.887 | −7.319 | −0.20 | −0.02 | −0.026 | 1.478 |
| ATOM | 9 | N12 | UDP | 9 | −1.264 | −12.977 | −5.321 | −0.46 | −0.20 | −0.292 | 1.478 |
| ATOM | 10 | H12 | UDP | 9 | −1.279 | −13.929 | −5.852 | −0.18 | +0.14 | +0.393 | 1.478 |
| ATOM | 11 | C9 | UDP | 9 | −1.296 | −13.030 | −3.928 | −0.69 | −0.02 | −0.023 | 1.478 |
| ATOM | 12 | O18 | UDP | 9 | −1.330 | −14.138 | −3.390 | −0.17 | −0.02 | −0.026 | 1.478 |
| ATOM | 13 | C7 | UDP | 9 | −1.260 | −11.757 | −3.280 | −0.47 | +0.00 | +0.000 | 1.478 |
| ATOM | 14 | C5 | UDP | 9 | −1.219 | −10.639 | −4.020 | −0.23 | −0.03 | −0.050 | 1.478 |
| ATOM | 15 | O21 | UDP | 9 | −3.344 | −8.491 | −6.361 | −0.26 | −0.59 | −0.537 | 1.478 |
| ATOM | 16 | H21 | UDP | 9 | −4.027 | −8.887 | −5.699 | +0.07 | +0.48 | +0.424 | 1.478 |
| ATOM | 17 | O23 | UDP | 9 | −1.758 | −6.919 | −7.623 | −0.10 | −0.10 | −0.537 | 1.478 |
| ATOM | 18 | H23 | UDP | 9 | −1.894 | −5.925 | −7.849 | −0.22 | −0.20 | +0.424 | 1.478 |
| ATOM | 19 | C25 | UDP | 9 | 0.771 | −6.645 | −4.973 | −0.34 | +0.04 | +0.113 | 1.478 |
| ATOM | 20 | O28 | UDP | 9 | 1.938 | −6.031 | −5.487 | +0.07 | −0.19 | −0.368 | 1.478 |
| ATOM | 21 | P39 | UDP | 9 | 2.381 | −4.527 | −5.672 | −0.36 | +0.89 | +1.210 | 1.478 |
| ATOM | 22 | O30 | UDP | 9 | 1.262 | −3.664 | −5.199 | −0.08 | −0.78 | −0.850 | 1.478 |
| ATOM | 23 | O31 | UDP | 9 | 3.728 | −4.347 | −5.046 | +0.01 | −0.93 | −0.850 | 1.478 |
| ATOM | 24 | O32 | UDP | 9 | 2.397 | −4.567 | −7.265 | +0.01 | −0.35 | −0.510 | 1.478 |
| ATOM | 25 | P33 | UDP | 9 | 3.574 | −4.543 | −8.347 | −0.71 | +1.27 | +1.210 | 1.478 |
| ATOM | 26 | O36 | UDP | 9 | 4.361 | −3.371 | −7.882 | −0.26 | −1.68 | −0.850 | 1.478 |
| ATOM | 27 | O35 | UDP | 9 | 3.115 | −4.626 | −9.757 | −0.21 | −0.20 | −0.255 | 1.478 |
| ATOM | 28 | O34 | UDP | 9 | 4.378 | −5.926 | −8.114 | −0.07 | −0.97 | −0.850 | 1.478 |
| TER | | | | | | | | | | | |

ENDMDL

| | |
|---|---|
| MODEL | 30 |

| USER | Run = 30 | | | | | | | | | | |
|---|---|---|---|---|---|---|---|---|---|---|---|
| USER | Cluster Rank = 9 | | | | | | | | | | |
| USER | Number of conformations in this cluster = 5 | | | | | | | | | | |
| USER | | | | | | | | | | | |
| USER | RMSD from reference structure | | | | | = 1.166 A | | | | | |
| USER | | | | | | | | | | | |
| USER | Estimated Free Energy of Binding | | | | | = −8.41 kcal/mol [= (1) + (3)] | | | | | |
| USER | Estimated Inhibition Constant, Ki | | | | | = +6.89e−07 [Temperature = 298.15 K] | | | | | |
| USER | | | | | | | | | | | |
| USER | Final Docked Energy | | | | | = −11.26 kcal/mol [= (1) + (2)] | | | | | |
| USER | | | | | | | | | | | |
| USER | (1) Final Intermolecular Energy | | | | | = −10.90 kcal/mol | | | | | |
| USER | (2) Final Internal Energy of Ligand | | | | | = −0.36 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.046941 −8.001134 −5.504904 | | | | | | | | | | |
| USER | NEWDPF quat0 −0.534048 −0.795776 0.285539 1.180865 | | | | | | | | | | |
| USER | NEWDPF ndihe8 | | | | | | | | | | |
| USER | NEWDPF dihe0 40.93 74.31 30.18 178.37 4.15 −20.95 −0.13 −151.79 | | | | | | | | | | |
| USER | | | | | | | | | | | |
| USER | | | | Rank | x | y | z | vdW | Elec | q | RMS |
| ATOM | 1 | C19 | UDP | 9 | −1.111 | −9.392 | −6.154 | −0.49 | +0.10 | +0.174 | 1.166 |
| ATOM | 2 | C1 | UDP | 9 | −2.103 | −8.326 | −5.689 | −0.39 | +0.07 | +0.113 | 1.166 |
| ATOM | 3 | C3 | UDP | 9 | −1.398 | −7.046 | −6.194 | −0.37 | +0.02 | +0.113 | 1.166 |
| ATOM | 4 | O16 | UDP | 9 | 0.146 | −8.810 | −5.829 | −0.02 | −0.12 | −0.227 | 1.166 |
| ATOM | 5 | C14 | UDP | 9 | 0.088 | −7.378 | −6.072 | −0.42 | +0.03 | +0.113 | 1.166 |
| ATOM | 6 | N10 | UDP | 9 | −1.183 | −10.658 | −5.413 | −0.34 | +0.03 | +0.039 | 1.166 |
| ATOM | 7 | C11 | UDP | 9 | −1.218 | −11.836 | −6.106 | −0.65 | −0.06 | −0.073 | 1.166 |
| ATOM | 8 | O17 | UDP | 9 | −1.190 | −11.867 | −7.338 | −0.20 | −0.02 | −0.026 | 1.166 |
| ATOM | 9 | N12 | UDP | 9 | −1.270 | −12.984 | −5.356 | −0.44 | −0.20 | −0.292 | 1.166 |
| ATOM | 10 | H12 | UDP | 9 | −1.282 | −13.928 | −5.901 | −0.16 | +0.13 | +0.393 | 1.166 |
| ATOM | 11 | C9 | UDP | 9 | −1.311 | −13.058 | −3.964 | −0.69 | −0.02 | −0.023 | 1.166 |
| ATOM | 12 | O18 | UDP | 9 | −1.349 | −14.173 | −3.442 | −0.17 | −0.02 | −0.026 | 1.166 |
| ATOM | 13 | C7 | UDP | 9 | −1.278 | −11.793 | −3.298 | −0.48 | +0.00 | +0.000 | 1.166 |
| ATOM | 14 | C5 | UDP | 9 | −1.232 | −10.665 | −4.022 | −0.24 | −0.03 | −0.050 | 1.166 |
| ATOM | 15 | O21 | UDP | 9 | −3.340 | −8.482 | −6.344 | −0.26 | −0.59 | −0.537 | 1.166 |
| ATOM | 16 | H21 | UDP | 9 | −4.095 | −8.539 | −5.646 | +0.07 | +0.55 | +0.424 | 1.166 |
| ATOM | 17 | O23 | UDP | 9 | −1.745 | −6.894 | −7.575 | −0.08 | −0.09 | −0.537 | 1.166 |
| ATOM | 18 | H23 | UDP | 9 | −2.068 | −5.932 | −7.744 | −0.19 | −0.17 | −0.424 | 1.166 |
| ATOM | 19 | C25 | UDP | 9 | 0.769 | −6.660 | −4.906 | −0.33 | +0.04 | +0.113 | 1.166 |
| ATOM | 20 | O28 | UDP | 9 | 0.595 | −5.268 | −5.094 | +0.09 | −0.10 | −0.368 | 1.166 |
| ATOM | 21 | P39 | UDP | 9 | 1.599 | −4.067 | −5.295 | −0.32 | +0.88 | +1.210 | 1.166 |
| ATOM | 22 | O30 | UDP | 9 | 0.808 | −2.804 | −5.257 | −0.26 | −1.66 | −0.850 | 1.166 |
| ATOM | 23 | O31 | UDP | 9 | 2.729 | −4.223 | −4.328 | +0.09 | −0.72 | −0.850 | 1.166 |
| ATOM | 24 | O32 | UDP | 9 | 1.970 | −4.427 | −6.803 | +0.03 | −0.28 | −0.510 | 1.166 |
| ATOM | 25 | P33 | UDP | 9 | 3.359 | −4.577 | −7.580 | −0.60 | +1.24 | +1.210 | 1.166 |
| ATOM | 26 | O36 | UDP | 9 | 4.031 | −3.305 | −7.205 | −0.29 | −1.40 | −0.850 | 1.166 |
| ATOM | 27 | O35 | UDP | 9 | 3.226 | −4.960 | −9.009 | −0.19 | −0.22 | −0.255 | 1.166 |
| ATOM | 28 | O34 | UDP | 9 | 4.079 | −5.852 | −6.897 | −0.05 | −0.96 | −0.850 | 1.166 |
| TER | | | | | | | | | | | |

ENDMDL

| | |
|---|---|
| MODEL | 32 |

| USER | Run = 32 | | | | | | | | | | |
|---|---|---|---|---|---|---|---|---|---|---|---|
| USER | Cluster Rank = 9 | | | | | | | | | | |
| USER | Number of conformations in this cluster = 5 | | | | | | | | | | |
| USER | | | | | | | | | | | |
| USER | RMSD from reference structure | | | | | = 1.337 A | | | | | |
| USER | | | | | | | | | | | |
| USER | Estimated Free Energy of Binding | | | | | = −8.45 kcal/mol [(1) + (3)] | | | | | |
| USER | Estimated Inhibition Constant, Ki | | | | | = 6.45e−07 [Temperature = 298.15 K] | | | | | |
| USER | | | | | | | | | | | |
| USER | Final Docked Energy | | | | | = −11.15 kcal/mol [= (1) + (2)] | | | | | |
| USER | | | | | | | | | | | |
| USER | (1) Final Intermolecular Energy | | | | | = −10.94 kcal/mol | | | | | |
| USER | (2) Final Internal Energy of Ligand | | | | | = −0.22 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.007618 −8.024051 −5.554527 | | | | | | | | | | |
| USER | NEWDPF quat0 0.038449 0.977474 −0.207523 4.243013 | | | | | | | | | | |
| USER | NEWDPF ndihe8 | | | | | | | | | | |
| USER | NEWDPF dihe0 57.72 87.38 31.32 −156.52 8.88 −34.37 −8.16 61.24 | | | | | | | | | | |
| USER | | | | | | | | | | | |
| USER | | | | Rank | x | y | z | vdW | Elec | q | RMS |
| ATOM | 1 | C19 | UDP | 9 | −1.160 | −9.403 | −6.213 | −0.50 | +0.10 | +0.174 | 1.337 |
| ATOM | 2 | C1 | UDP | 9 | −2.083 | −8.324 | −5.648 | −0.39 | +0.07 | +0.113 | 1.337 |
| ATOM | 3 | C3 | UDP | 9 | −1.397 | −7.051 | −6.198 | −0.38 | +0.02 | +0.113 | 1.337 |
| ATOM | 4 | O16 | UDP | 9 | 0.134 | −8.853 | −5.994 | −0.03 | −0.12 | −0.227 | 1.337 |
| ATOM | 5 | C14 | UDP | 9 | 0.086 | −7.416 | −6.212 | −0.43 | +0.03 | +0.113 | 1.337 |
| ATOM | 6 | N10 | UDP | 9 | −1.194 | −10.679 | −5.485 | −0.34 | +0.03 | +0.039 | 1.337 |
| ATOM | 7 | C11 | UDP | 9 | −1.218 | −11.849 | −6.190 | −0.63 | −0.06 | −0.073 | 1.337 |
| ATOM | 8 | O17 | UDP | 9 | −1.211 | −11.866 | −7.423 | −0.21 | −0.02 | −0.026 | 1.337 |
| ATOM | 9 | N12 | UDP | 9 | −1.235 | −13.006 | −5.452 | −0.40 | −0.20 | −0.292 | 1.337 |
| ATOM | 10 | H12 | UDP | 9 | −1.238 | −13.945 | −6.007 | −0.15 | +0.11 | +0.393 | 1.337 |
| ATOM | 11 | C9 | UDP | 9 | −1.250 | −13.095 | −4.060 | −0.71 | −0.02 | −0.023 | 1.337 |
| ATOM | 12 | O18 | UDP | 9 | −1.257 | −14.217 | −3.550 | −0.20 | −0.02 | −0.026 | 1.337 |
| ATOM | 13 | C7 | UDP | 9 | −1.230 | −11.838 | −3.381 | −0.49 | +0.00 | +0.000 | 1.337 |
| ATOM | 14 | C5 | UDP | 9 | −1.219 | −10.701 | −4.094 | −0.26 | −0.03 | −0.050 | 1.337 |
| ATOM | 15 | O21 | UDP | 9 | −3.376 | −8.444 | −6.193 | −0.24 | −0.61 | −0.537 | 1.337 |
| ATOM | 16 | H21 | UDP | 9 | −4.076 | −8.215 | −5.473 | +0.07 | +0.61 | +0.424 | 1.337 |
| ATOM | 17 | O23 | UDP | 9 | −1.861 | −6.872 | −7.540 | −0.10 | −0.10 | −0.537 | 1.337 |
| ATOM | 18 | H23 | UDP | 9 | −1.969 | −5.868 | −7.735 | −0.16 | −0.25 | +0.424 | 1.337 |
| ATOM | 19 | C25 | UDP | 9 | 0.883 | −6.730 | −5.102 | −0.35 | +0.04 | +0.113 | 1.337 |
| ATOM | 20 | O28 | UDP | 9 | 0.880 | −5.339 | −5.364 | +0.08 | −0.11 | −0.368 | 1.337 |
| ATOM | 21 | P39 | UDP | 9 | 2.010 | −4.237 | −5.388 | −0.34 | +0.88 | +1.210 | 1.337 |
| ATOM | 22 | O30 | UDP | 9 | 1.400 | −2.953 | −4.938 | −0.27 | −1.16 | −0.850 | 1.337 |
| ATOM | 23 | O31 | UDP | 9 | 3.198 | −4.756 | −4.643 | +0.06 | −0.76 | −0.850 | 1.337 |
| ATOM | 24 | O32 | UDP | 9 | 2.181 | −4.228 | −6.973 | +0.01 | −0.33 | −0.510 | 1.337 |
| ATOM | 25 | P33 | UDP | 9 | 3.454 | −4.336 | −7.935 | −0.65 | +1.31 | +1.210 | 1.337 |
| ATOM | 26 | O36 | UDP | 9 | 4.342 | −3.301 | −7.344 | −0.27 | −1.68 | −0.850 | 1.337 |
| ATOM | 27 | O35 | UDP | 9 | 3.133 | −4.308 | −9.385 | −0.19 | −0.21 | −0.255 | 1.337 |
| ATOM | 28 | O34 | UDP | 9 | 4.032 | −5.824 | −7.689 | −0.09 | −0.92 | −0.850 | 1.337 |
| TER | | | | | | | | | | | |

ENDMDL

| | |
|---|---|
| MODEL | 18 |

| USER | Run = 18 | | | | | | | | | | |
|---|---|---|---|---|---|---|---|---|---|---|---|
| USER | Cluster Rank = 9 | | | | | | | | | | |
| USER | Number of conformations in this cluster = 5 | | | | | | | | | | |
| USER | | | | | | | | | | | |
| USER | RMSD from reference structure | | | | | = 1.488 A | | | | | |
| USER | | | | | | | | | | | |
| USER | Estimated Free Energy of Binding | | | | | = −7.78 kcal/mol [= (1) + (3)] | | | | | |
| USER | Estimated Inhibition Constant, Ki | | | | | = +1.97e−06 [Temperature = 298.15 K.] | | | | | |
| USER | | | | | | | | | | | |
| USER | Final Docked Energy | | | | | = −10.89 kcal/mol [= (1) + (2)] | | | | | |
| USER | | | | | | | | | | | |
| USER | (1) Final Intermolecular Energy | | | | | = −10.27 kcal/mol | | | | | |
| USER | (2) Final Internal Energy of Ligand | | | | | = −0.62 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.933032 −7.725676 −5.704241 | | | | | | | | | | |
| USER | NEWDPF quat0 −0.183935 −0.981472 0.053677 21.916061 | | | | | | | | | | |
| USER | NEWDPF ndihe 8 | | | | | | | | | | |
| USER | NEWDPF dihe0 23.10 75.74 50.86 22.04 −13.62 180.00 −65.54 −176.89 | | | | | | | | | | |
| USER | | | | | | | | | | | |
| USER | | | | Rank | x | y | z | vdW | Elec | q | RMS |
| ATOM | 1 | C19 | UDP | 9 | −0.757 | −9.151 | −6.250 | −0.50 | +0.10 | +0.174 | 1.488 |
| ATOM | 2 | C1 | UDP | 9 | −1.855 | −8.088 | −6.227 | −0.44 | +0.06 | +0.113 | 1.488 |
| ATOM | 3 | C3 | UDP | 9 | −1.025 | −6.819 | −6.529 | +0.03 | +0.00 | +0.113 | 1.488 |
| ATOM | 4 | O16 | UDP | 9 | 0.303 | −8.519 | −5.540 | +0.00 | −0.11 | −0.227 | 1.488 |
| ATOM | 5 | C14 | UDP | 9 | 0.325 | −7.105 | −5.874 | −0.39 | +0.03 | +0.113 | 1.488 |
| ATOM | 6 | N10 | UDP | 9 | −1.078 | −10.376 | −5.506 | −0.32 | +0.03 | +0.039 | 1.488 |
| ATOM | 7 | C11 | UDP | 9 | −1.216 | −11.553 | −6.187 | −0.68 | −0.06 | −0.073 | 1.488 |
| ATOM | 8 | O17 | UDP | 9 | −1.077 | −11.617 | −7.411 | −0.25 | −0.02 | −0.026 | 1.488 |
| ATOM | 9 | N12 | UDP | 9 | −1.501 | −12.665 | −5.434 | −0.44 | −0.21 | −0.292 | 1.488 |
| ATOM | 10 | H12 | UDP | 9 | −1.597 | −13.610 | −5.968 | −0.25 | +0.08 | +0.393 | 1.488 |
| ATOM | 11 | C9 | UDP | 9 | −1.682 | −12.699 | −4.052 | −0.60 | −0.02 | −0.023 | 1.488 |
| ATOM | 12 | O18 | UDP | 9 | −1.928 | −13.785 | −3.525 | −0.09 | −0.02 | −0.026 | 1.488 |
| ATOM | 13 | C7 | UDP | 9 | −1.532 | −11.436 | −3.399 | −0.44 | +0.00 | +0.000 | 1.488 |
| ATOM | 14 | C5 | UDP | 9 | −1.258 | −10.343 | −4.127 | −0.26 | −0.03 | −0.050 | 1.488 |
| ATOM | 15 | O21 | UDP | 9 | −2.781 | −8.311 | −7.264 | −0.23 | −0.41 | −0.537 | 1.488 |
| ATOM | 16 | H21 | UDP | 9 | −3.671 | −8.644 | −6.865 | +0.07 | +0.46 | +0.424 | 1.488 |
| ATOM | 17 | O23 | UDP | 9 | −0.864 | −6.751 | −7.950 | −0.04 | −0.01 | −0.537 | 1.488 |
| ATOM | 18 | H23 | UDP | 9 | −1.090 | −5.802 | −8.276 | −0.27 | −0.25 | +0.424 | 1.488 |
| ATOM | 19 | C25 | UDP | 9 | 0.548 | −6.307 | −4.588 | −0.30 | +0.04 | +0.113 | 1.488 |
| ATOM | 20 | O28 | UDP | 9 | 1.745 | −5.566 | −4.741 | +0.10 | −0.20 | −0.368 | 1.488 |
| ATOM | 21 | P39 | UDP | 9 | 2.089 | −4.192 | −5.438 | −0.35 | +0.90 | +1.210 | 1.488 |
| ATOM | 22 | O30 | UDP | 9 | 0.886 | −3.318 | −5.333 | −0.27 | −1.01 | −0.850 | 1.488 |
| ATOM | 23 | O31 | UDP | 9 | 3.382 | −3.686 | −4.881 | +0.03 | −0.88 | −0.850 | 1.488 |
| ATOM | 24 | O32 | UDP | 9 | 2.197 | −4.790 | −6.911 | +0.03 | −0.30 | −0.510 | 1.488 |
| ATOM | 25 | P33 | UDP | 9 | 3.424 | −5.017 | −7.910 | −0.65 | +1.17 | +1.210 | 1.488 |
| ATOM | 26 | O36 | UDP | 9 | 4.064 | −3.676 | −7.882 | −0.33 | −1.36 | −0.850 | 1.488 |
| ATOM | 27 | O35 | UDP | 9 | 3.054 | −5.643 | −9.206 | −0.17 | −0.22 | −0.255 | 1.488 |
| ATOM | 28 | O34 | UDP | 9 | 4.345 | −6.130 | −7.186 | +0.01 | −1.00 | −0.850 | 1.488 |
| TER | | | | | | | | | | | |

ENDMDL

| | |
|---|---|
| MODEL | 13 |

| USER | Run = 13 | | | | | | | | | | |
|---|---|---|---|---|---|---|---|---|---|---|---|
| USER | Cluster Rank = 9 | | | | | | | | | | |
| USER | Number of conformations in this cluster = 5 | | | | | | | | | | |
| USER | | | | | | | | | | | |
| USER | RMSD from reference structure | | | | | = 1.129 A | | | | | |
| USER | | | | | | | | | | | |
| USER | Estimated Free Energy of Binding | | | | | = −8.12 kcal/mol [= (1) + (3)] | | | | | |
| USER | Estimated Inhibition Constant, Ki | | | | | = +1.11e−06 [Temperature = 298.15 K] | | | | | |
| USER | | | | | | | | | | | |
| USER | Final Docked Energy | | | | | = −10.82 kcal/mol [= (1) + (2)] | | | | | |
| USER | | | | | | | | | | | |
| USER | (1) Final Intermolecular Energy | | | | | = −10.61 kcal/mol | | | | | |
| USER | (2) Final Internal Energy of Ligand | | | | | = −0.20 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.302796 −7.926836 −5.187975 | | | | | | | | | | |
| USER | NEWDPF quat0 0.777049 0.235348 0.583786 6.172968 | | | | | | | | | | |
| USER | NEWDPF ndihe 8 | | | | | | | | | | |
| USER | NEWDPF dihe0 34.05 44.04 −38.20 179.83 4.99 −20.08 −14.04 178.49 | | | | | | | | | | |
| USER | | | | | | | | | | | |
| USER | | | | Rank | x | y | z | vdW | Elec | q | RMS |
| ATOM | 1 | C19 | UDP | 9 | −1.319 | −9.253 | −5.963 | −0.46 | +0.10 | +0.174 | 1.129 |
| ATOM | 2 | C1 | UDP | 9 | −2.347 | −8.296 | −5.360 | −0.37 | +0.07 | +0.113 | 1.129 |
| ATOM | 3 | C3 | UDP | 9 | −1.737 | −6.934 | −5.769 | −0.35 | +0.03 | +0.113 | 1.129 |
| ATOM | 4 | O16 | UDP | 9 | −0.082 | −8.630 | −5.635 | +0.01 | −0.11 | −0.227 | 1.129 |
| ATOM | 5 | C14 | UDP | 9 | −0.231 | −7.187 | −5.738 | −0.36 | +0.03 | +0.113 | 1.129 |
| ATOM | 6 | N10 | UDP | 9 | −1.287 | −10.584 | −5.344 | −0.32 | +0.03 | +0.039 | 1.129 |
| ATOM | 7 | C11 | UDP | 9 | −1.270 | −11.692 | −6.144 | −0.66 | −0.06 | −0.073 | 1.129 |
| ATOM | 8 | O17 | UDP | 9 | −1.280 | −11.607 | −7.374 | −0.27 | −0.02 | −0.026 | 1.129 |
| ATOM | 9 | N12 | UDP | 9 | −1.226 | −12.906 | −5.505 | −0.41 | −0.21 | −0.292 | 1.129 |
| ATOM | 10 | H12 | UDP | 9 | −1.196 | −13.795 | −6.136 | −0.13 | +0.11 | +0.393 | 1.129 |
| ATOM | 11 | C9 | UDP | 9 | −1.217 | −13.111 | −4.126 | −0.72 | −0.02 | −0.023 | 1.129 |
| ATOM | 12 | O18 | UDP | 9 | −1.169 | −14.270 | −3.711 | −0.24 | −0.02 | −0.026 | 1.129 |
| ATOM | 13 | C7 | UDP | 9 | −1.242 | −11.914 | −3.344 | −0.52 | +0.00 | +0.000 | 1.129 |
| ATOM | 14 | C5 | UDP | 9 | −1.290 | −10.723 | −3.959 | −0.26 | −0.03 | −0.050 | 1.129 |
| ATOM | 15 | O21 | UDP | 9 | −3.601 | −8.463 | −5.978 | −0.16 | −0.66 | −0.537 | 1.129 |
| ATOM | 16 | H21 | UDP | 9 | −4.297 | −8.743 | −5.273 | +0.07 | +0.51 | +0.424 | 1.129 |
| ATOM | 17 | O23 | UDP | 9 | −2.153 | −6.676 | −7.114 | −0.15 | −0.13 | −0.537 | 1.129 |
| ATOM | 18 | H23 | UDP | 9 | −2.943 | −6.017 | −7.109 | −0.06 | +0.19 | +0.424 | 1.129 |
| ATOM | 19 | C25 | UDP | 9 | 0.461 | −6.541 | −4.536 | −0.30 | +0.04 | +0.113 | 1.129 |
| ATOM | 20 | O28 | UDP | 9 | 0.483 | −5.142 | −4.751 | +0.08 | −0.13 | −0.368 | 1.129 |
| ATOM | 21 | P39 | UDP | 9 | 1.646 | −4.088 | −4.914 | −0.31 | +0.94 | +1.210 | 1.129 |
| ATOM | 22 | O30 | UDP | 9 | 1.032 | −2.730 | −4.896 | −0.31 | −1.66 | −0.850 | 1.129 |
| ATOM | 23 | O31 | UDP | 9 | 2.712 | −4.396 | −3.911 | +0.09 | −0.68 | −0.850 | 1.129 |
| ATOM | 24 | O32 | UDP | 9 | 2.014 | −4.496 | −6.410 | +0.05 | −0.30 | −0.510 | 1.129 |
| ATOM | 25 | P33 | UDP | 9 | 2.802 | −3.749 | −7.584 | −0.58 | +1.12 | +1.210 | 1.129 |
| ATOM | 26 | O36 | UDP | 9 | 2.110 | −2.433 | −7.595 | −0.09 | −0.88 | −0.850 | 1.129 |
| ATOM | 27 | O35 | UDP | 9 | 2.931 | −4.537 | −8.836 | −0.14 | −0.21 | −0.255 | 1.129 |
| ATOM | 28 | O34 | UDP | 9 | 4.319 | −3.617 | −7.044 | −0.18 | −1.60 | −0.850 | 1.129 |
| TER | | | | | | | | | | | |

ENDMDL

| | |
|---|---|
| MODEL | 3 |

| USER | Run = 3 | | | | | | | | | | |
|---|---|---|---|---|---|---|---|---|---|---|---|
| USER | Cluster Rank = 10 | | | | | | | | | | |
| USER | Number of conformations in this cluster = 1 | | | | | | | | | | |
| USER | | | | | | | | | | | |
| USER | RMSD from reference structure | | | | | = 2.420 A | | | | | |
| USER | | | | | | | | | | | |
| USER | Estimated Free Energy of Binding | | | | | = −8.29 kcal/mol [= (1) + (3)] | | | | | |
| USER | Estimated Inhibition Constant, Ki | | | | | = +8.34e−07 [Temperature = 298.15 K] | | | | | |
| USER | | | | | | | | | | | |
| USER | Final Docked Energy | | | | | = −11.12 kcal/mol [= (1) + (2)] | | | | | |
| USER | | | | | | | | | | | |
| USER | (1) Final Intermolecular Energy | | | | | = −10.78 kcal/mol | | | | | |
| USER | (2) Final Internal Energy of Ligand | | | | | = −0.34 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.045885 −7.584484 −4.823250 | | | | | | | | | | |
| USER | NEWDPF quat0 −0.810371 −0.575655 0.109182 −79.293267 | | | | | | | | | | |
| USER | NEWDPF ndihe 8. | | | | | | | | | | |
| USER | NEWDPF dihe0 37.74 40.69 86.97 −109.13 −6.73 −57.52 −25.38 −167.24 | | | | | | | | | | |
| USER | | | | | | | | | | | |
| USER | | | | Rank | x | y | z | vdW | Elec | q | RMS |
| ATOM | 1 | C19 | UDP | 10 | −1.107 | −7.675 | −5.930 | −0.40 | +0.06 | +0.174 | 2.420 |
| ATOM | 2 | C1 | UDP | 10 | −1.049 | −7.874 | −4.416 | −0.30 | +0.04 | +0.113 | 2.420 |
| ATOM | 3 | C3 | UDP | 10 | −0.165 | −6.672 | −4.008 | −0.27 | +0.04 | +0.113 | 2.420 |
| ATOM | 4 | O16 | UDP | 10 | 0.237 | −7.333 | −6.252 | +0.03 | −0.07 | −0.227 | 2.420 |
| ATOM | 5 | C14 | UDP | 10 | 0.785 | −6.502 | −5.192 | −0.34 | +0.04 | +0.113 | 2.420 |
| ATOM | 6 | N10 | UDP | 10 | −1.416 | −8.891 | −6.695 | −0.28 | +0.02 | +0.039 | 2.420 |
| ATOM | 7 | C11 | UDP | 10 | −2.515 | −8.907 | −7.507 | −0.59 | −0.05 | −0.073 | 2.420 |
| ATOM | 8 | O17 | UDP | 10 | −3.256 | −7.927 | −7.619 | −0.29 | −0.02 | −0.026 | 2.420 |
| ATOM | 9 | N12 | UDP | 10 | −2.742 | −10.067 | −8.205 | −0.41 | −0.17 | −0.292 | 2.420 |
| ATOM | 10 | H12 | UDP | 10 | −3.607 | −10.082 | −8.867 | +0.02 | +0.18 | +0.393 | 2.420 |
| ATOM | 11 | C9 | UDP | 10 | −1.971 | −11.228 | −8.145 | −0.85 | −0.01 | −0.023 | 2.420 |
| ATOM | 12 | O18 | UDP | 10 | −2.308 | −12.185 | −8.844 | −0.40 | −0.01 | −0.026 | 2.420 |
| ATOM | 13 | C7 | UDP | 10 | −0.841 | −11.150 | −7.274 | −0.71 | +0.00 | +0.000 | 2.420 |
| ATOM | 14 | C5 | UDP | 10 | −0.614 | −10.022 | −6.584 | −0.64 | −0.03 | −0.050 | 2.420 |
| ATOM | 15 | O21 | UDP | 10 | −2.331 | −7.739 | −3.850 | +0.12 | −0.24 | −0.537 | 2.420 |
| ATOM | 16 | H21 | UDP | 10 | −2.568 | −8.593 | −3.326 | +0.11 | +0.20 | +0.424 | 2.420 |
| ATOM | 17 | O23 | UDP | 10 | −1.026 | −5.532 | −3.916 | +0.09 | −0.14 | −0.537 | 2.420 |
| ATOM | 18 | H23 | UDP | 10 | −1.301 | −5.387 | −2.935 | +0.11 | +0.12 | +0.424 | 2.420 |
| ATOM | 19 | C25 | UDP | 10 | 2.208 | −6.977 | −4.891 | −0.47 | +0.06 | +0.113 | 2.420 |
| ATOM | 20 | O28 | UDP | 10 | 2.884 | −5.923 | −4.230 | +0.04 | −0.27 | −0.368 | 2.420 |
| ATOM | 21 | P39 | UDP | 10 | 4.407 | −5.534 | −4.086 | −0.64 | +1.45 | +1.210 | 2.420 |
| ATOM | 22 | O30 | UDP | 10 | 4.872 | −6.011 | −2.752 | −0.45 | −1.45 | −0.850 | 2.420 |
| ATOM | 23 | O31 | UDP | 10 | 5.137 | −6.007 | −5.301 | −0.04 | −1.25 | −0.850 | 2.420 |
| ATOM | 24 | O32 | UDP | 10 | 4.159 | −3.960 | −4.040 | −0.04 | −0.59 | −0.510 | 2.420 |
| ATOM | 25 | P33 | UDP | 10 | 3.771 | −2.882 | −5.156 | −0.58 | +1.45 | +1.210 | 2.420 |
| ATOM | 26 | O36 | UDP | 10 | 4.782 | −3.185 | −6.203 | −0.03 | −1.79 | −0.850 | 2.420 |
| ATOM | 27 | O35 | UDP | 10 | 3.615 | −1.496 | −4.644 | −0.12 | −0.27 | −0.255 | 2.420 |
| ATOM | 28 | O34 | UDP | 10 | 2.283 | −3.296 | −5.630 | +0.03 | −0.81 | −0.850 | 2.420 |
| TER | | | | | | | | | | | |

ENDMDL

| | |
|---|---|
| MODEL | 5 |

| USER | Run = 5 | | | | | | | | | | |
|---|---|---|---|---|---|---|---|---|---|---|---|
| USER | Cluster Rank = 11 | | | | | | | | | | |
| USER | Number of conformations in this cluster = 2 | | | | | | | | | | |
| USER | | | | | | | | | | | |
| USER | RMSD from reference structure | | | | | = 2.081 A | | | | | |
| USER | | | | | | | | | | | |
| USER | Estimated Free Energy of Binding | | | | | = −8.44 kcal/mol [= (1) + (3)] | | | | | |
| USER | Estimated Inhibition Constant, Ki | | | | | = +6.52e−07 [Temperature = 298.15 K] | | | | | |
| USER | | | | | | | | | | | |
| USER | Final Docked Energy | | | | | = −10.98 kcal/mol [= (1) + (2)] | | | | | |
| USER | | | | | | | | | | | |
| USER | (1) Final Intermolecular Energy | | | | | = −10.93 kcal/mol | | | | | |
| USER | (2) Final Internal Energy of Ligand | | | | | = −0.05 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.012566 −8.074526 −5.325992 | | | | | | | | | | |
| USER | NEWDPF quat0 0.706358 −0.697817 0.118782 0.464725 | | | | | | | | | | |
| USER | NEWDPF ndihe 8 | | | | | | | | | | |
| USER | NEWDPF dihe0 −5.40 87.91 3.44 172.65 5.48 −104.32 −116.53 −123.56 | | | | | | | | | | |
| USER | | | | | | | | | | | |
| USER | | | | Rank | x | y | z | vdW | Elec | q | RMS |
| ATOM | 1 | C19 | UDP | 11 | −1.090 | −9.454 | −5.997 | −0.49 | +0.10 | +0.174 | 2.081 |
| ATOM | 2 | C1 | UDP | 11 | −2.072 | −8.392 | −5.504 | −0.38 | +0.07 | +0.113 | 2.081 |
| ATOM | 3 | C3 | UDP | 11 | −1.367 | −7.106 | −5.995 | −0.38 | +0.03 | +0.113 | 2.081 |
| ATOM | 4 | O16 | UDP | 11 | 0.173 | −8.883 | −5.677 | −0.01 | −0.12 | −0.227 | 2.081 |
| ATOM | 5 | C14 | UDP | 11 | 0.119 | −7.447 | −5.895 | −0.41 | +0.04 | +0.113 | 2.081 |
| ATOM | 6 | N10 | UDP | 11 | −1.160 | −10.732 | −5.277 | −0.34 | +0.03 | +0.039 | 2.081 |
| ATOM | 7 | C11 | UDP | 11 | −1.181 | −11.899 | −5.989 | −0.64 | −0.06 | −0.073 | 2.081 |
| ATOM | 8 | O17 | UDP | 11 | −1.141 | −11.910 | −7.221 | −0.17 | −0.02 | −0.026 | 2.081 |
| ATOM | 9 | N12 | UDP | 11 | −1.233 | −13.059 | −5.257 | −0.49 | −0.20 | −0.292 | 2.081 |
| ATOM | 10 | H12 | UDP | 11 | −1.233 | −13.995 | −5.817 | −0.19 | +0.16 | +0.393 | 2.081 |
| ATOM | 11 | C9 | UDP | 11 | −1.286 | −13.155 | −3.867 | −0.69 | −0.02 | −0.023 | 2.081 |
| ATOM | 12 | O18 | UDP | 11 | −1.322 | −14.278 | −3.363 | −0.16 | −0.02 | −0.026 | 2.081 |
| ATOM | 13 | C7 | UDP | 11 | −1.268 | −11.901 | −3.181 | −0.51 | +0.00 | +0.000 | 2.081 |
| ATOM | 14 | C5 | UDP | 11 | −1.222 | −10.761 | −3.887 | −0.20 | −0.03 | −0.050 | 2.081 |
| ATOM | 15 | O21 | UDP | 11 | −3.317 | −8.530 | −6.149 | −0.25 | −0.57 | −0.537 | 2.081 |
| ATOM | 16 | H21 | UDP | 11 | −3.855 | −9.284 | −5.699 | +0.08 | +0.42 | +0.424 | 2.081 |
| ATOM | 17 | O23 | UDP | 11 | −1.727 | −6.930 | −7.370 | −0.07 | −0.09 | −0.537 | 2.081 |
| ATOM | 18 | H23 | UDP | 11 | −1.827 | −5.926 | −7.572 | −0.21 | −0.29 | +0.424 | 2.081 |
| ATOM | 19 | C25 | UDP | 11 | 0.816 | −6.752 | −4.724 | −0.34 | +0.04 | +0.113 | 2.081 |
| ATOM | 20 | O28 | UDP | 11 | 2.199 | −7.040 | −4.812 | −0.01 | −0.20 | −0.368 | 2.081 |
| ATOM | 21 | P39 | UDP | 11 | 3.492 | −6.332 | −4.248 | −0.56 | +1.02 | +1.210 | 2.081 |
| ATOM | 22 | O30 | UDP | 11 | 4.675 | −7.104 | −4.725 | −0.23 | −0.94 | −0.850 | 2.081 |
| ATOM | 23 | O31 | UDP | 11 | 3.419 | −4.875 | −4.577 | +0.04 | −0.80 | −0.850 | 2.081 |
| ATOM | 24 | O32 | UDP | 11 | 3.202 | −6.676 | −2.720 | −0.02 | −0.36 | −0.510 | 2.081 |
| ATOM | 25 | P33 | UDP | 11 | 3.687 | −6.025 | −1.342 | −0.61 | +1.07 | +1.210 | 2.081 |
| ATOM | 26 | O36 | UDP | 11 | 5.157 | −5.971 | −1.558 | −0.40 | −1.81 | −0.850 | 2.081 |
| ATOM | 27 | O35 | UDP | 11 | 3.132 | −6.674 | −0.126 | −0.09 | −0.14 | −0.255 | 2.081 |
| ATON | 28 | O34 | UDP | 11 | 3.035 | −4.547 | −1.332 | +0.06 | −0.57 | −0.850 | 2.081 |
| TER | | | | | | | | | | | |

ENDMDL

| | |
|---|---|
| MODEL | 28 |

| USER | Run = 28 | | | | | | | | | | |
|---|---|---|---|---|---|---|---|---|---|---|---|
| USER | Cluster Rank = 11 | | | | | | | | | | |
| USER | Number of conformations in this cluster = 2 | | | | | | | | | | |
| USER | | | | | | | | | | | |
| USER | RMSD from reference structure | | | | | = 2.009 A | | | | | |
| USER | | | | | | | | | | | |
| USER | Estimated Free Energy of Binding | | | | | = −8.27 kcal/mol [= (1) + (3)] | | | | | |
| USER | Estimated Inhibition Constant, Ki | | | | | = +8.63e−07 [Temperature = 298.15 K] | | | | | |
| USER | | | | | | | | | | | |
| USER | Final Docked Energy | | | | | = −10.64 kcal/mol [= (1) + (2)] | | | | | |
| USER | | | | | | | | | | | |
| USER | (1) Final Intermolecular Energy | | | | | = −10.76 kcal/mol | | | | | |
| USER | (2) Final Internal Energy of Ligand | | | | | = +0.13 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.048830 −8.018897 −5.305900 | | | | | | | | | | |
| USER | NEWDPF quat0 0.073887 −0.957739 −0.277987 −0.254508 | | | | | | | | | | |
| USER | NEWDPF ndihe 8 | | | | | | | | | | |
| USER | NEWDPF dihe0 30.49 117.86 −5.93 105.75 4.25 −52.96 −74.13 −107.76 | | | | | | | | | | |
| USER | | | | | | | | | | | |
| USER | | | | Rank | x | y | z | vdW | Elec | q | RMS |
| ATOM | 1 | C19 | UDP | 11 | −1.133 | −9.402 | −5.968 | −0.48 | +0.10 | +0.174 | 2.009 |
| ATOM | 2 | C1 | UDP | 11 | −2.110 | −8.337 | −5.471 | −0.37 | +0.07 | +0.113 | 2.009 |
| ATOM | 3 | C3 | UDP | 11 | −1.410 | −7.055 | −5.978 | −0.38 | +0.03 | +0.113 | 2.009 |
| ATOM | 4 | O16 | UDP | 11 | 0.134 | −8.830 | −5.664 | −0.01 | −0.12 | −0.227 | 2.009 |
| ATOM | 5 | C14 | UDP | 11 | 0.077 | −7.395 | −5.890 | −0.40 | +0.03 | +0.113 | 2.009 |
| ATOM | 6 | N10 | UDP | 11 | −1.195 | −10.676 | −5.239 | −0.33 | +0.03 | +0.039 | 2.009 |
| ATOM | 7 | C11 | UDP | 11 | −1.249 | −11.846 | −5.944 | −0.67 | −0.06 | −0.073 | 2.009 |
| ATOM | 8 | O17 | UDP | 11 | −1.244 | −11.864 | −7.177 | −0.23 | −0.02 | −0.026 | 2.009 |
| ATOM | 9 | N12 | UDP | 11 | −1.291 | −13.002 | −5.205 | −0.48 | −0.21 | −0.292 | 2.009 |
| ATOM | 10 | H12 | UDP | 11 | −1.317 | −13.941 | −5.760 | −0.19 | +0.15 | +0.393 | 2.009 |
| ATOM | 11 | C9 | UDP | 11 | −1.305 | −13.090 | −3.814 | −0.68 | −0.02 | −0.023 | 2.009 |
| ATOM | 12 | O18 | UDP | 11 | −1.338 | −14.211 | −3.303 | −0.14 | −0.02 | −0.026 | 2.009 |
| ATOM | 13 | C7 | UDP | 11 | −1.255 | −11.833 | −3.135 | −0.49 | +0.00 | +0.000 | 2.009 |
| ATOM | 14 | C5 | UDP | 11 | −1.218 | −10.697 | −3.848 | −0.18 | −0.03 | −0.050 | 2.009 |
| ATOM | 15 | O21 | UDP | 11 | −3.361 | −8.480 | −6.103 | −0.24 | −0.59 | −0.537 | 2.009 |
| ATOM | 16 | H21 | UDP | 11 | −4.076 | −8.716 | −5.400 | +0.07 | +0.49 | +0.424 | 2.009 |
| ATOM | 17 | O23 | UDP | 11 | −1.784 | −6.886 | −7.349 | −0.07 | −0.09 | −0.537 | 2.009 |
| ATOM | 18 | H23 | UDP | 11 | −1.417 | −5.991 | −7.698 | −0.24 | −0.31 | +0.424 | 2.009 |
| ATOM | 19 | C25 | UDP | 11 | 0.785 | −6.692 | −4.730 | −0.33 | +0.04 | +0.113 | 2.009 |
| ATOM | 20 | O28 | UDP | 11 | 1.975 | −6.116 | −5.236 | +0.07 | −0.19 | −0.368 | 2.009 |
| ATOM | 21 | P39 | UDP | 11 | 3.381 | −5.792 | −4.596 | −0.50 | +1.03 | +1.210 | 2.009 |
| ATOM | 22 | O30 | UDP | 11 | 4.395 | −6.648 | −5.275 | −0.14 | −0.89 | −0.850 | 2.009 |
| ATOM | 23 | O31 | UDP | 11 | 3.582 | −4.311 | −4.610 | +0.02 | −0.87 | −0.850 | 2.009 |
| ATOM | 24 | O32 | UDP | 11 | 3.038 | −6.395 | −3.161 | −0.03 | −0.36 | −0.510 | 2.009 |
| ATOM | 25 | P33 | UDP | 11 | 3.758 | −6.301 | −1.736 | −0.55 | +1.13 | +1.210 | 2.009 |
| ATOM | 26 | O36 | UDP | 11 | 5.151 | −6.671 | −2.099 | −0.54 | −1.69 | −0.850 | 2.009 |
| ATOM | 27 | O35 | UDP | 11 | 3.053 | −7.018 | −0.642 | −0.06 | −0.13 | −0.255 | 2.009 |
| ATOM | 28 | O34 | UDP | 11 | 3.647 | −4.744 | −1.320 | +0.04 | −0.75 | −0.850 | 2.009 |
| TER | | | | | | | | | | | |

ENDMDL

| | |
|---|---|
| MODEL | 41 |

| USER | Run = 41 | | | | | | | | | | |
|---|---|---|---|---|---|---|---|---|---|---|---|
| USER | Cluster Rank = 12 | | | | | | | | | | |
| USER | Number of conformations in this cluster = 1 | | | | | | | | | | |
| USER | | | | | | | | | | | |
| USER | RMSD from reference structure | | | | | = 2.122 A | | | | | |
| USER | | | | | | | | | | | |
| USER | Estimated Free Energy of Binding | | | | | = −8.78 kcal/mol [= (1) + (3)] | | | | | |
| USER | Estimated Inhibition Constant, Ki | | | | | = +3.64e−07 [Temperature = 298.15 K] | | | | | |
| USER | | | | | | | | | | | |
| USER | Final Docked Energy | | | | | = −10.75 kcal/mol [= (1) + (2)] | | | | | |
| USER | | | | | | | | | | | |
| USER | (1) Final Intermolecular Energy | | | | | = −11.27 kcal/mol | | | | | |
| USER | (2) Final Internal Energy of Ligand | | | | | = +0.52 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 0.614825 −7.020692 −6.505622 | | | | | | | | | | |
| USER | NEWDPF quat0 0.444788 0.808872 0.384564 −35.773088 | | | | | | | | | | |
| USER | NEWDPF ndihe 8 | | | | | | | | | | |
| USER | NEWDPF dihe0 45.04 77.48 87.45 −0.60 176.90 −18.84 −2.33 50.25 | | | | | | | | | | |
| USER | | | | | | | | | | | |
| USER | | | | Rank | x | y | z | vdW | Elec | q | RMS |
| ATOM | 1 | C19 | UDP | 12 | 0.432 | −8.512 | −6.826 | −0.60 | +0.09 | +0.174 | 2.122 |
| ATOM | 2 | C1 | UDP | 12 | −0.303 | −7.205 | −7.120 | −0.47 | +0.02 | +0.113 | 2.122 |
| ATOM | 3 | C3 | UDP | 12 | −0.891 | −6.277 | −7.445 | −0.46 | +0.02 | +0.113 | 2.122 |
| ATOM | 4 | O16 | UDP | 12 | −1.537 | −8.078 | −6.041 | −0.08 | −0.11 | −0.227 | 2.122 |
| ATOM | 5 | C14 | UDP | 12 | −2.010 | −6.800 | −6.547 | −0.49 | +0.06 | +0.113 | 2.122 |
| ATOM | 6 | N10 | UDP | 12 | −0.319 | −9.460 | −5.992 | −0.33 | +0.02 | +0.039 | 2.122 |
| ATOM | 7 | C11 | UDP | 12 | −0.796 | −9.050 | −4.779 | −0.28 | −0.04 | −0.073 | 2.122 |
| ATOM | 8 | O17 | UDP | 12 | −0.617 | −7.903 | −4.363 | +0.08 | −0.01 | −0.026 | 2.122 |
| ATOM | 9 | N12 | UDP | 12 | −1.501 | −9.978 | −4.054 | −0.22 | −0.17 | −0.292 | 2.122 |
| ATOM | 10 | H12 | UDP | 12 | −1.900 | −9.654 | −3.093 | +0.10 | +0.19 | +0.393 | 2.122 |
| ATOM | 11 | C9 | UDP | 12 | −1.755 | −11.297 | −4.431 | −0.48 | −0.02 | −0.023 | 2.122 |
| ATOM | 12 | O18 | UDP | 12 | −2.413 | −12.003 | −3.665 | +0.00 | −0.02 | −0.026 | 2.122 |
| ATOM | 13 | C7 | UDP | 12 | −1.224 | −11.666 | −5.705 | −0.66 | +0.00 | +0.000 | 2.122 |
| ATOM | 14 | C5 | UDP | 12 | −0.529 | −10.768 | −6.419 | −0.68 | −0.04 | −0.050 | 2.122 |
| ATOM | 15 | O21 | UDP | 12 | −1.123 | −7.344 | −8.257 | −0.01 | −0.16 | −0.537 | 2.122 |
| ATOM | 16 | H21 | UDP | 12 | −2.068 | −6.993 | −8.045 | +0.01 | +0.14 | +0.424 | 2.122 |
| ATOM | 17 | O23 | UDP | 12 | 1.233 | −6.499 | −8.817 | −0.14 | −0.26 | −0.537 | 2.122 |
| ATOM | 18 | H23 | UDP | 12 | 1.363 | −5.594 | −9.289 | +0.05 | +0.20 | +0.424 | 2.122 |
| ATOM | 19 | C25 | UDP | 12 | 2.301 | −5.885 | −5.356 | −0.39 | +0.07 | +0.113 | 2.122 |
| ATOM | 20 | O28 | UDP | 12 | 2.691 | −4.623 | −5.866 | +0.07 | −0.29 | −0.368 | 2.122 |
| ATOM | 21 | P39 | UDP | 12 | 4.025 | −3.784 | −5.780 | −0.55 | +1.66 | +1.210 | 2.122 |
| ATOM | 22 | O30 | UDP | 12 | 5.068 | −4.653 | −5.164 | −0.09 | −1.45 | −0.850 | 2.122 |
| ATOM | 23 | O31 | UDP | 12 | 4.302 | −3.191 | −7.125 | −0.26 | −1.63 | −0.850 | 2.122 |
| ATOM | 24 | O32 | UDP | 12 | 3.482 | −2.743 | −4.703 | −0.01 | −0.55 | −0.510 | 2.122 |
| ATOM | 25 | P33 | UDP | 12 | 2.458 | −1.518 | −4.786 | −0.63 | +1.47 | +1.210 | 2.122 |
| ATOM | 26 | O36 | UDP | 12 | 3.004 | −0.772 | −5.950 | −0.08 | −1.24 | −0.850 | 2.122 |
| ATOM | 27 | O35 | UDP | 12 | 2.224 | −0.823 | −3.494 | −0.09 | −0.20 | −0.255 | 2.122 |
| ATOM | 28 | O34 | UDP | 12 | 1.032 | −2.195 | −5.131 | −0.40 | −1.92 | −0.850 | 2.122 |
| TER | | | | | | | | | | | |

ENDMDL

| | |
|---|---|
| MODEL | 47 |

| USER | Run = 47 | | | | | | | | | | |
|---|---|---|---|---|---|---|---|---|---|---|---|
| USER | Cluster Rank = 13 | | | | | | | | | | |
| USER | Number of conformations in this cluster = 1 | | | | | | | | | | |
| USER | | | | | | | | | | | |
| USER | RMSD from reference structure | | | | | = 8.313 A | | | | | |
| USER | | | | | | | | | | | |
| USER | Estimated Free Energy of Binding | | | | | = −8.36 kcal/mol [= (1) + (3)] | | | | | |
| USER | Estimated Inhibition Constant, Ki | | | | | = +7.47e−07 [Temperature = 298.15 K] | | | | | |
| USER | | | | | | | | | | | |
| USER | Final Docked Energy | | | | | = −10.74 kcal/mol [= (1) + (2)] | | | | | |
| USER | | | | | | | | | | | |
| USER | (1) Final Intermolecular Energy | | | | | = −10.85 kcal/mol | | | | | |
| USER | (2) Final Internal Energy of Ligand | | | | | = +0.11 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.521974 −0.726960 −0.369204 | | | | | | | | | | |
| USER | NEWDPF quat0 0.912797 0.056531 −0.404483 −168.985079 | | | | | | | | | | |
| USER | NEWDPF ndihe 8 | | | | | | | | | | |
| USER | NEWDPF dihe0 10.54 21.96 −94.72 180.00 −12.32 168.27 −123.42 57.24 | | | | | | | | | | |
| USER | | | | | | | | | | | |
| USER | | | | Rank | x | y | z | vdW | Elec | q | RMS |
| ATOM | 1 | C19 | UDP | 13 | −1.120 | 0.521 | 0.430 | −0.45 | −0.08 | +0.174 | 8.313 |
| ATOM | 2 | C1 | UDP | 13 | −2.114 | −0.630 | 0.577 | −0.51 | −0.07 | +0.113 | 8.313 |
| ATOM | 3 | C3 | UDP | 13 | −1.237 | −1.819 | 0.119 | −0.31 | −0.03 | +0.113 | 8.313 |
| ATOM | 4 | O16 | UDP | 13 | −0.488 | 0.232 | −0.811 | +0.05 | +0.02 | −0.227 | 8.313 |
| ATOM | 5 | C14 | UDP | 13 | −0.321 | −1.207 | −0.939 | −0.31 | +0.01 | +0.113 | 8.313 |
| ATOM | 6 | N10 | UDP | 13 | −1.737 | 1.846 | 0.280 | −0.39 | −0.02 | +0.039 | 8.313 |
| ATOM | 7 | C11 | UDP | 13 | −1.533 | 2.791 | 1.246 | −0.59 | +0.07 | −0.073 | 8.313 |
| ATOM | 8 | O17 | UDP | 13 | −0.843 | 2.564 | 2.243 | −0.02 | +0.03 | −0.026 | 8.313 |
| ATOM | 9 | N12 | UDP | 13 | −2.125 | 4.012 | 1.041 | −0.50 | +0.25 | −0.292 | 8.313 |
| ATOM | 10 | H12 | UDP | 13 | −1.954 | 4.776 | 1.799 | +0.06 | −0.41 | +0.393 | 8.313 |
| ATOM | 11 | C9 | UDP | 13 | −2.925 | 4.367 | −0.045 | −0.85 | +0.01 | −0.023 | 8.313 |
| ATOM | 12 | O18 | UDP | 13 | −3.377 | 5.512 | −0.083 | −0.30 | +0.02 | −0.026 | 8.313 |
| ATOM | 13 | C7 | UDP | 13 | −3.105 | 3.334 | −1.017 | −0.82 | +0.00 | +0.000 | 8.313 |
| ATOM | 14 | C5 | UDP | 13 | −2.535 | 2.136 | −0.822 | −0.67 | +0.02 | −0.050 | 8.313 |
| ATOM | 15 | O21 | UDP | 13 | −2.480 | −0.799 | 1.926 | −0.09 | +0.59 | −0.537 | 8.313 |
| ATOM | 16 | H21 | UDP | 13 | −3.365 | −0.303 | 2.107 | −0.25 | −0.64 | +0.424 | 8.313 |
| ATOM | 17 | O23 | UDP | 13 | −0.460 | −2.227 | 1.249 | +0.16 | +0.10 | −0.537 | 8.313 |
| ATOM | 18 | H23 | UDP | 13 | −1.044 | −2.788 | 1.883 | +0.11 | −0.11 | +0.424 | 8.313 |
| ATOM | 19 | C25 | UDP | 13 | −0.695 | −1.614 | −2.365 | −0.35 | +0.11 | +0.113 | 8.313 |
| ATOM | 20 | O28 | UDP | 13 | 0.199 | −0.964 | −3.249 | −0.16 | −0.66 | −0.368 | 8.313 |
| ATOM | 21 | P39 | UDP | 13 | 1.735 | −1.142 | −3.564 | −0.50 | +1.30 | +1.210 | 8.313 |
| ATOM | 22 | O30 | UDP | 13 | 2.155 | −0.007 | −4.433 | −0.23 | −0.94 | −0.850 | 8.313 |
| ATOM | 23 | O31 | UDP | 13 | 2.464 | −1.354 | −2.275 | +0.05 | −0.34 | −0.850 | 8.313 |
| ATOM | 24 | O32 | UDP | 13 | 1.559 | −2.460 | −4.443 | −0.24 | −0.78 | −0.510 | 8.313 |
| ATOM | 25 | P33 | UDP | 13 | 1.603 | −2.737 | −6.017 | −0.50 | +1.31 | +1.210 | 8.313 |
| ATOM | 26 | O36 | UDP | 13 | 0.667 | −1.694 | −6.513 | −0.55 | −1.23 | −0.850 | 8.313 |
| ATOM | 27 | O35 | UDP | 13 | 1.407 | −4.160 | −6.397 | +0.03 | −0.11 | −0.255 | 8.313 |
| ATOM | 28 | O34 | UDP | 13 | 3.127 | −2.421 | −6.450 | −0.08 | −1.04 | −0.850 | 8.313 |
| TER | | | | | | | | | | | |

ENDMDL

| | |
|---|---|
| MODEL | 11 |

| USER | Run = 11 | | | | | | | | | | |
|---|---|---|---|---|---|---|---|---|---|---|---|
| USER | Cluster Rank = 14 | | | | | | | | | | |
| USER | Number of conformations in this cluster = 1 | | | | | | | | | | |
| USER | | | | | | | | | | | |
| USER | RMSD from reference structure | | | | | = 9.257 A | | | | | |
| USER | | | | | | | | | | | |
| USER | Estimated Free Energy of Binding | | | | | = −8.26 kcal/mol [=(1) + (3)] | | | | | |
| USER | Estimated Inhibition Constant, Ki | | | | | = +8.86e−07 [Temperature = 298.15 K] | | | | | |
| USER | | | | | | | | | | | |
| USER | Final Docked Energy | | | | | = −10.21 kcal/mol [=(1) + (2)] | | | | | |
| USER | | | | | | | | | | | |
| USER | (1) Final Intermolecular Energy | | | | | = −10.75 kcal/mol | | | | | |
| USER | (2) Final Internal Energy of Ligand | | | | | = +0.54 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −1.074714 1.274345 0.654726 | | | | | | | | | | |
| USER | NEWDPF quat0 −0.659616 0.137486 0.738921 −150.105716 | | | | | | | | | | |
| USER | NEWDPF ndihe 8 | | | | | | | | | | |
| USER | NEWDPF dihe0 50.00 55.60 −31.11 12.36 −178.25 180.00 −120.60 179.70 | | | | | | | | | | |
| USER | | | | | | | | | | | |
| USER | | | | Rank | x | y | z | vdW | Elec | q | RMS |
| ATOM | 1 | C19 | UDP | 14 | −0.697 | 2.561 | −0.093 | −0.54 | −0.09 | +0.174 | 9.257 |
| ATOM | 2 | C1 | UDP | 14 | −0.913 | 2.132 | 1.357 | −0.47 | −0.10 | +0.113 | 9.257 |
| ATOM | 3 | C3 | UDP | 14 | −0.205 | 0.757 | 1.353 | −0.31 | −0.06 | +0.113 | 9.257 |
| ATOM | 4 | O16 | UDP | 14 | −0.956 | 1.363 | −0.815 | −0.03 | +0.05 | −0.227 | 9.257 |
| ATOM | 5 | C14 | UDP | 14 | −0.446 | 0.230 | −0.061 | −0.35 | −0.03 | +0.113 | 9.257 |
| ATOM | 6 | N10 | UDP | 14 | −1.659 | 3.556 | −0.587 | −0.44 | −0.02 | +0.039 | 9.257 |
| ATOM | 7 | C11 | UDP | 14 | −2.996 | 3.335 | −0.410 | −0.79 | +0.03 | −0.073 | 9.257 |
| ATOM | 8 | O17 | UDP | 14 | −3.420 | 2.324 | 0.155 | −0.13 | +0.01 | −0.026 | 9.257 |
| ATOM | 9 | N12 | UDP | 14 | −3.843 | 4.308 | −0.880 | −0.55 | +0.13 | −0.292 | 9.257 |
| ATOM | 10 | H12 | UDP | 14 | −4.910 | 4.148 | −0.728 | −0.14 | −0.31 | +0.393 | 9.257 |
| ATOM | 11 | C9 | UDP | 14 | −3.467 | 5.481 | −1.534 | −0.96 | +0.01 | −0.023 | 9.257 |
| ATOM | 12 | O18 | UDP | 14 | −4.354 | 6.258 | −1.889 | −0.21 | +0.01 | −0.026 | 9.257 |
| ATOM | 13 | C7 | UDP | 14 | −2.057 | 5.650 | −1.694 | −0.85 | +0.00 | +0.000 | 9.257 |
| ATOM | 14 | C5 | UDP | 14 | −1.221 | 4.702 | −1.243 | −0.55 | +0.02 | −0.050 | 9.257 |
| ATOM | 15 | O21 | UDP | 14 | −0.250 | 3.009 | 2.237 | −0.02 | +0.66 | −0.537 | 9.257 |
| ATOM | 16 | H21 | UDP | 14 | −0.872 | 3.241 | 3.025 | −0.01 | −0.72 | +0.424 | 9.257 |
| ATOM | 17 | O23 | UDP | 14 | 1.193 | 1.006 | 1.541 | +0.14 | +0.20 | −0.537 | 9.257 |
| ATOM | 18 | H23 | UDP | 14 | 1.483 | 0.650 | 2.461 | +0.11 | −0.16 | +0.424 | 9.257 |
| ATOM | 19 | C25 | UDP | 14 | −1.483 | −0.893 | −0.112 | −0.42 | −0.04 | +0.113 | 9.257 |
| ATOM | 20 | O28 | UDP | 14 | −1.593 | −1.318 | −1.458 | −0.01 | +0.02 | −0.368 | 9.257 |
| ATOM | 21 | P39 | UDP | 14 | −0.749 | −2.303 | −2.357 | −0.24 | +1.11 | +1.210 | 9.257 |
| ATOM | 22 | O30 | UDP | 14 | 0.000 | −3.217 | −1.448 | +0.16 | −0.32 | −0.850 | 9.257 |
| ATOM | 23 | O31 | UDP | 14 | −1.655 | −2.910 | −3.380 | −0.36 | −1.03 | −0.850 | 9.257 |
| ATOM | 24 | O32 | UDP | 14 | 0.233 | −1.186 | −2.929 | −0.28 | −0.75 | −0.510 | 9.257 |
| ATOM | 25 | P33 | UDP | 14 | 1.607 | −1.252 | −3.744 | −0.46 | +1.54 | +1.210 | 9.257 |
| ATOM | 26 | O36 | UDP | 14 | 2.386 | −2.200 | −2.906 | +0.06 | −0.61 | −0.850 | 9.257 |
| ATOM | 27 | O35 | UDP | 14 | 2.175 | 0.074 | −4.101 | −0.18 | −0.22 | −0.255 | 9.257 |
| ATOM | 28 | O34 | UDP | 14 | 1.211 | −1.905 | −5.168 | −0.48 | −1.77 | −0.850 | 9.257 |
| TER | | | | | | | | | | | |

ENDMDL

| | |
|---|---|
| MODEL | 7 |

| USER | Run = 7 | | | | | | | | | | |
|---|---|---|---|---|---|---|---|---|---|---|---|
| USER | Cluster Rank = 15 | | | | | | | | | | |
| USER | Number of conformations in this cluster = 1 | | | | | | | | | | |
| USER | | | | | | | | | | | |
| USER | RMSD from reference structure | | | | | = 8.035 A | | | | | |
| USER | | | | | | | | | | | |
| USER | Estimated Free Energy of Binding | | | | | = −9.09 kcal/mol [=(1) + (3)] | | | | | |
| USER | Estimated Inhibition Constant, Ki | | | | | = +2.18e−07 [Temperature = 298.15 K] | | | | | |
| USER | | | | | | | | | | | |
| USER | Final Docked Energy | | | | | = −10.07 kcal/mol [=(1) + (2)] | | | | | |
| USER | | | | | | | | | | | |
| USER | (1) Final Intermolecular Energy | | | | | = −11.58 kcal/mol | | | | | |
| USER | (2) Final Internal Energy of Ligand | | | | | = +1.51 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.323475 −0.046387 −1.524826 | | | | | | | | | | |
| USER | NEWDPF quat0 0.962843 0.192599 −0.189314 −162.709260 | | | | | | | | | | |
| USER | NEWDPF ndihe 8 | | | | | | | | | | |
| USER | NEWDPF dihe0 40.34 30.33 −46.04 −179.95 62.45 −78.60 10.17 179.76 | | | | | | | | | | |
| USER | | | | | | | | | | | |
| USER | | | | Rank | x | y | z | vdW | Elec | q | RMS |
| ATOM | 1 | C19 | UDP | 15 | −0.544 | −0.997 | −0.419 | −0.43 | −0.04 | +0.174 | 8.035 |
| ATOM | 2 | C1 | UDP | 15 | −1.260 | −0.248 | −0.944 | −0.48 | −0.01 | +0.113 | 8.035 |
| ATOM | 3 | C3 | UDP | 15 | −0.056 | −1.192 | −1.168 | −0.30 | +0.02 | +0.113 | 8.035 |
| ATOM | 4 | O16 | UDP | 15 | 0.588 | 1.090 | −1.276 | +0.03 | +0.00 | −0.227 | 8.035 |
| ATOM | 5 | C14 | UDP | 15 | 1.075 | −0.248 | −1.573 | −0.36 | +0.03 | +0.113 | 8.035 |
| ATOM | 6 | N10 | UDP | 15 | −1.293 | 2.249 | −0.588 | −0.37 | −0.01 | +0.039 | 8.035 |
| ATOM | 7 | C11 | UDP | 15 | −0.662 | 3.440 | −0.358 | −0.56 | +0.04 | −0.073 | 8.035 |
| ATOM | 8 | O17 | UDP | 15 | 0.521 | 3.494 | −0.015 | +0.02 | +0.02 | −0.026 | 8.035 |
| ATOM | 9 | N12 | UDP | 15 | −1.411 | 4.574 | −0.547 | −0.32 | +0.20 | −0.292 | 8.035 |
| ATOM | 10 | H12 | UDP | 15 | −0.911 | 5.528 | −0.382 | +0.03 | −0.56 | +0.393 | 8.035 |
| ATOM | 11 | C9 | UDP | 15 | −2.752 | 4.621 | −0.928 | −0.89 | +0.01 | −0.023 | 8.035 |
| ATOM | 12 | O18 | UDP | 15 | −3.280 | 5.727 | −1.061 | −0.29 | +0.01 | −0.026 | 8.035 |
| ATOM | 13 | C7 | UDP | 15 | −3.356 | 3.344 | −1.146 | −0.75 | +0.00 | +0.000 | 8.035 |
| ATOM | 14 | C5 | UDP | 15 | −2.634 | 2.230 | −0.959 | −0.63 | +0.02 | −0.050 | 8.035 |
| ATOM | 15 | O21 | UDP | 15 | −2.110 | −0.783 | 0.043 | −0.05 | +0.28 | −0.537 | 8.035 |
| ATOM | 16 | H21 | UDP | 15 | −3.062 | −0.879 | −0.338 | −0.27 | −0.37 | +0.424 | 8.035 |
| ATOM | 17 | O23 | UDP | 15 | 0.248 | −1.790 | 0.097 | +0.17 | +0.00 | −0.537 | 8.035 |
| ATOM | 18 | H23 | UDP | 15 | −0.328 | −2.632 | 0.226 | +0.11 | −0.01 | +0.424 | 8.035 |
| ATOM | 19 | C25 | UDP | 15 | 1.412 | −0.321 | −3.063 | −0.22 | +0.08 | +0.113 | 8.035 |
| ATOM | 20 | O28 | UDP | 15 | 1.531 | −1.688 | −3.412 | −0.16 | −0.44 | −0.368 | 8.035 |
| ATOM | 21 | P39 | UDP | 15 | 1.925 | −2.437 | −4.744 | −0.46 | +1.56 | +1.210 | 8.035 |
| ATOM | 22 | O30 | UDP | 15 | 1.649 | −3.889 | −4.546 | +0.05 | −0.74 | −0.850 | 8.035 |
| ATOM | 23 | O31 | UDP | 15 | 1.267 | −1.743 | −5.894 | −0.59 | −1.45 | −0.850 | 8.035 |
| ATOM | 24 | O32 | UDP | 15 | 3.489 | −2.161 | −4.619 | −0.05 | −0.54 | −0.510 | 8.035 |
| ATOM | 25 | P33 | UDP | 15 | 4.782 | −2.940 | −5.147 | −0.65 | +1.95 | +1.210 | 8.035 |
| ATOM | 26 | O36 | UDP | 15 | 4.492 | −4.320 | −4.675 | −0.10 | −1.14 | −0.850 | 8.035 |
| ATOM | 27 | O35 | UDP | 15 | 6.074 | −2.289 | −4.808 | −0.41 | −0.55 | −0.255 | 8.035 |
| ATOM | 28 | O34 | UDP | 15 | 4.699 | −2.834 | −6.757 | −0.13 | −1.89 | −0.850 | 8.035 |
| TER | | | | | | | | | | | |

ENDMDL

| | |
|---|---|
| MODEL | 19 |

| USER | Run = 19 | | | | | | | | | | |
|---|---|---|---|---|---|---|---|---|---|---|---|
| USER | Cluster Rank = 16 | | | | | | | | | | |
| USER | Number of conformations in this cluster = 1 | | | | | | | | | | |
| USER | | | | | | | | | | | |
| USER | RMSD from reference structure | | | | | = 4.207 A | | | | | |
| USER | | | | | | | | | | | |
| USER | Estimated Free Energy of Binding | | | | | = −7.01 kcal/mol [=(1) + (3)] | | | | | |
| USER | Estimated Inhibition Constant, Ki | | | | | = +7.33e−06 [Temperature = 298.15 K] | | | | | |
| USER | | | | | | | | | | | |
| USER | Final Docked Energy | | | | | = −9.69 kcal/mol [=(1) + (2)] | | | | | |
| USER | | | | | | | | | | | |
| USER | (1) Final Intermolecular Energy | | | | | = −9.50 kcal/mol | | | | | |
| USER | (2) Final Internal Energy of Ligand | | | | | = −0.20 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 −0.979803 −4.544452 −2.904171 | | | | | | | | | | |
| USER | NEWDPF quat0 0.536869 0.454790 −0.710589 95.326407 | | | | | | | | | | |
| USER | NEWDPF ndihe 8 | | | | | | | | | | |
| USER | NEWDPF dihe0 −6.06 40.91 −28.38 30.29 6.49 −154.36 −81.51 22.93 | | | | | | | | | | |
| USER | | | | | | | | | | | |
| USER | | | | Rank | x | y | z | vdW | Elec | q | RMS |
| ATOM | 1 | C19 | UDP | 16 | −2.370 | −4.104 | −3.387 | −0.42 | +0.04 | +0.174 | 4.207 |
| ATOM | 2 | C1 | UDP | 16 | −1.530 | −3.968 | −2.117 | −0.27 | +0.03 | +0.113 | 4.207 |
| ATOM | 3 | C3 | UDP | 16 | −0.143 | −3.661 | −2.728 | −0.23 | +0.09 | +0.113 | 4.207 |
| ATOM | 4 | O16 | UDP | 16 | −1.521 | −4.861 | −4.242 | +0.02 | −0.04 | −0.227 | 4.207 |
| ATOM | 5 | C14 | UDP | 16 | −0.143 | −4.445 | −4.039 | −0.26 | +0.07 | +0.113 | 4.207 |
| ATOM | 6 | N10 | UDP | 16 | −3.600 | −4.890 | −3.226 | −0.30 | +0.00 | +0.039 | 4.207 |
| ATOM | 7 | C11 | UDP | 16 | −4.768 | −4.419 | −3.757 | −0.56 | +0.00 | −0.073 | 4.207 |
| ATOM | 8 | O17 | UDP | 16 | −4.819 | −3.351 | −4.371 | +0.25 | +0.00 | −0.026 | 4.207 |
| ATOM | 9 | N12 | UDP | 16 | −5.878 | −5.206 | −3.585 | −0.43 | +0.02 | −0.292 | 4.207 |
| ATOM | 10 | H12 | UDP | 16 | −6.811 | −4.846 | −4.019 | −0.26 | −0.18 | +0.393 | 4.207 |
| ATOM | 11 | C9 | UDP | 16 | −5.928 | −6.423 | −2.905 | −0.54 | −0.01 | −0.023 | 4.207 |
| ATOM | 12 | O18 | UDP | 16 | −7.008 | −7.012 | −2.846 | −0.17 | −0.01 | −0.026 | 4.207 |
| ATOM | 13 | C7 | UDP | 16 | −4.677 | −6.858 | −2.367 | −0.30 | +0.00 | +0.000 | 4.207 |
| ATOM | 14 | C5 | UDP | 16 | −3.586 | −6.093 | −2.526 | −0.27 | −0.01 | −0.050 | 4.207 |
| ATOM | 15 | O21 | UDP | 16 | −1.968 | −2.872 | −1.349 | +0.12 | +0.08 | −0.537 | 4.207 |
| ATOM | 16 | H21 | UDP | 16 | −2.809 | −3.137 | −0.818 | −0.16 | −0.22 | +0.424 | 4.207 |
| ATOM | 17 | O23 | UDP | 16 | −0.109 | −2.258 | −3.007 | −0.28 | −1.11 | −0.537 | 4.207 |
| ATOM | 18 | H23 | UDP | 16 | 0.363 | −1.766 | −2.237 | +0.10 | +0.36 | +0.424 | 4.207 |
| ATOM | 19 | C25 | UDP | 16 | 0.738 | −5.694 | −3.990 | −0.26 | +0.05 | +0.113 | 4.207 |
| ATOM | 20 | O28 | UDP | 16 | 1.420 | −5.791 | −5.227 | +0.09 | −0.16 | −0.368 | 4.207 |
| ATOM | 21 | P39 | UDP | 16 | 2.920 | −5.563 | −5.660 | −0.43 | +0.92 | +1.210 | 4.207 |
| ATOM | 22 | O30 | UDP | 16 | 3.791 | −6.113 | −4.583 | −0.05 | −0.80 | −0.850 | 4.207 |
| ATOM | 23 | O31 | UDP | 16 | 3.100 | −6.084 | −7.050 | −0.02 | −0.66 | −0.850 | 4.207 |
| ATOM | 24 | O32 | UDP | 16 | 2.870 | −3.973 | −5.556 | +0.06 | −0.47 | −0.510 | 4.207 |
| ATOM | 25 | P33 | UDP | 16 | 3.993 | −2.838 | −5.623 | −0.62 | +1.64 | +1.210 | 4.207 |
| ATOM | 26 | O36 | UDP | 16 | 4.978 | −3.358 | −4.639 | −0.17 | −1.33 | −0.850 | 4.207 |
| ATOM | 27 | O35 | UDP | 16 | 3.474 | −1.451 | −5.501 | −0.16 | −0.31 | −0.255 | 4.207 |
| ATOM | 28 | O34 | UDP | 16 | 4.566 | −2.912 | −7.132 | −0.07 | −1.90 | −0.850 | 4.207 |
| TER | | | | | | | | | | | |

ENDMDL

| | |
|---|---|
| MODEL | 45 |

| USER | Run = 45 | | | | | | | | | | |
|---|---|---|---|---|---|---|---|---|---|---|---|
| USER | Cluster Rank = 17 | | | | | | | | | | |
| USER | Number of conformations in this cluster = 1 | | | | | | | | | | |
| USER | | | | | | | | | | | |
| USER | RMSD from reference structure | | | | | = 6.471 A | | | | | |
| USER | | | | | | | | | | | |
| USER | Estimated Free Energy of Binding | | | | | = −7.96 kcal/mol [=(1) + (3)] | | | | | |
| USER | Estimated Inhibition Constant, Ki | | | | | = +1.47e−06 [Temperature = 298.15 K] | | | | | |
| USER | | | | | | | | | | | |
| USER | Final Docked Energy | | | | | = −9.26 kcal/mol [=(1) + (2)] | | | | | |
| USER | | | | | | | | | | | |
| USER | (1) Final Intermolecular Energy | | | | | = −10.45 kcal/mol | | | | | |
| USER | (2) Final Internal Energy of Ligand | | | | | = +1.19 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 2.363581 −2.545108 −3.968424 | | | | | | | | | | |
| USER | NEWDPF quat0 −0.722216 0.139035 −0.677549 146.873699 | | | | | | | | | | |
| USER | NEWDPF ndihe 8 | | | | | | | | | | |
| USER | NEWDPF dihe0 55.50 79.14 142.50 −48.91 −165.73 −80.26 −108.82 −24.10 | | | | | | | | | | |
| USER | | | | | | | | | | | |
| USER | | | | Rank | x | y | z | vdW | Elec | q | RMS |
| ATOM | 1 | C19 | UDP | 17 | 1.450 | −1.537 | −3.256 | −0.42 | +0.20 | +0.174 | 6.471 |
| ATOM | 2 | C1 | UDP | 17 | 2.011 | −1.740 | −4.663 | −0.56 | +0.15 | +0.113 | 6.471 |
| ATOM | 3 | C3 | UDP | 17 | 1.843 | −3.270 | −4.814 | −0.37 | +0.12 | +0.113 | 6.471 |
| ATOM | 4 | O16 | UDP | 17 | 2.012 | −2.628 | −2.536 | +0.09 | −0.16 | −0.227 | 6.471 |
| ATOM | 5 | C14 | UDP | 17 | 2.051 | −3.799 | −3.396 | −0.29 | +0.09 | +0.113 | 6.471 |
| ATOM | 6 | N10 | UDP | 17 | 1.915 | −0.317 | −2.583 | −0.32 | +0.02 | +0.039 | 6.471 |
| ATOM | 7 | C11 | UDP | 17 | 3.207 | 0.092 | −2.761 | −0.54 | −0.02 | −0.073 | 6.471 |
| ATOM | 8 | O17 | UDP | 17 | 3.996 | −0.534 | −3.473 | −0.08 | −0.01 | −0.026 | 6.471 |
| ATOM | 9 | N12 | UDP | 17 | 3.576 | 1.247 | −2.117 | −0.44 | +0.02 | −0.292 | 6.471 |
| ATOM | 10 | H12 | UDP | 17 | 4.601 | 1.589 | −2.267 | −0.27 | −0.10 | +0.393 | 6.471 |
| ATOM | 11 | C9 | UDP | 17 | 2.766 | 2.023 | −1.289 | −0.54 | +0.00 | −0.023 | 6.471 |
| ATOM | 12 | O18 | UDP | 17 | 3.250 | 3.040 | −0.790 | −0.16 | +0.00 | −0.026 | 6.471 |
| ATOM | 13 | C7 | UDP | 17 | 1.429 | 1.540 | −1.138 | −0.50 | +0.00 | +0.000 | 6.471 |
| ATOM | 14 | C5 | UDP | 17 | 1.060 | 0.409 | −1.759 | −0.33 | −0.01 | −0.050 | 6.471 |
| ATOM | 15 | O21 | UDP | 17 | 1.212 | −1.076 | −5.614 | −0.74 | −1.18 | −0.537 | 6.471 |
| ATOM | 16 | H21 | UDP | 17 | 1.815 | −0.654 | −6.335 | +0.05 | +1.00 | +0.424 | 6.471 |
| ATOM | 17 | O23 | UDP | 17 | 0.491 | −3.506 | −5.221 | −0.21 | −0.64 | −0.537 | 6.471 |
| ATOM | 18 | H23 | UDP | 17 | 0.478 | −4.199 | −5.981 | −0.17 | −0.02 | +0.424 | 6.471 |
| ATOM | 19 | C25 | UDP | 17 | 3.401 | −4.494 | −3.208 | −0.32 | +0.10 | +0.113 | 6.471 |
| ATOM | 20 | O28 | UDP | 17 | 3.356 | −5.200 | −1.981 | −0.03 | −0.31 | −0.368 | 6.471 |
| ATOM | 21 | P39 | UDP | 17 | 4.408 | −6.074 | −1.194 | −0.59 | +1.54 | +1.210 | 6.471 |
| ATOM | 22 | O30 | UDP | 17 | 5.157 | −6.890 | −2.192 | −0.38 | −1.72 | −0.850 | 6.471 |
| ATOM | 23 | O31 | UDP | 17 | 5.186 | −5.179 | −0.284 | −0.35 | −1.30 | −0.850 | 6.471 |
| ATOM | 24 | O32 | UDP | 17 | 3.328 | −6.984 | −0.454 | −0.06 | −0.29 | −0.510 | 6.471 |
| ATOM | 25 | P33 | UDP | 17 | 1.765 | −6.809 | −0.167 | −0.37 | +0.34 | +1.210 | 6.471 |
| ATOM | 26 | O36 | UDP | 17 | 1.737 | −5.445 | 0.423 | +0.14 | −0.25 | −0.850 | 6.471 |
| ATOM | 27 | O35 | UDP | 17 | 1.141 | −7.949 | 0.553 | −0.01 | −0.03 | −0.255 | 6.471 |
| ATOM | 28 | O34 | UDP | 17 | 1.071 | −6.839 | −1.626 | +0.03 | −0.26 | −0.850 | 6.471 |
| TER | | | | | | | | | | | |

ENDMDL

| | |
|---|---|
| MODEL | 37 |

| USER | Run = 37 | | | | | | | | | | |
|---|---|---|---|---|---|---|---|---|---|---|---|
| USER | Cluster Rank = 18 | | | | | | | | | | |
| USER | Number of conformations in this cluster = 1 | | | | | | | | | | |
| USER | | | | | | | | | | | |
| USER | RMSD from reference structure | | | | | = 5.907 A | | | | | |
| USER | | | | | | | | | | | |
| USER | Estimated Free Energy of Binding | | | | | = −5.38 kcal/mol [=(1) + (3)] | | | | | |
| USER | Estimated Inhibition Constant, Ki | | | | | = +1.13e−04 [Temperature = 298.15 K] | | | | | |
| USER | | | | | | | | | | | |
| USER | Final Docked Energy | | | | | = −7.90 kcal/mol [=(1) + (2)] | | | | | |
| USER | | | | | | | | | | | |
| USER | (1) Final Intermolecular Energy | | | | | = −7.87 kcal/mol | | | | | |
| USER | (2) Final Internal Energy of Ligand | | | | | = −0.02 kcal/mol | | | | | |
| USER | (3) Torsional Free Energy | | | | | = +2.49 kcal/mol | | | | | |
| USER | | | | | | | | | | | |
| USER | | | | | | | | | | | |
| USER | DPF = ligand_3.dpf | | | | | | | | | | |
| USER | NEWDPF move ligand.pdbq | | | | | | | | | | |
| USER | NEWDPF about −1.222000 −8.102000 −5.172000 | | | | | | | | | | |
| USER | NEWDPF tran0 2.658573 −5.712394 −1.136308 | | | | | | | | | | |
| USER | NEWDPF quat0 0.602972 0.510813 −0.612777 −175.487763 | | | | | | | | | | |
| USER | NEWDPF ndihe 8 | | | | | | | | | | |
| USER | NEWDPF dihe0 −8.75 31.27 −8.80 82.55 −9.84 28.21 −33.98 65.05 | | | | | | | | | | |
| USER | | | | | | | | | | | |
| USER | | | | Rank | x | y | z | vdW | Elec | q | RMS |
| ATOM | 1 | C19 | UDP | 18 | 2.413 | −4.726 | 0.015 | −0.25 | +0.07 | +0.174 | 5.907 |
| ATOM | 2 | C1 | UDP | 18 | 2.899 | −6.167 | −0.141 | −0.46 | +0.06 | +0.113 | 5.907 |
| ATOM | 3 | C3 | UDP | 18 | 3.826 | −6.020 | −1.370 | −0.63 | +0.11 | +0.113 | 5.907 |
| ATOM | 4 | O16 | UDP | 18 | 2.152 | −4.337 | −1.328 | +0.12 | −0.11 | −0.227 | 5.907 |
| ATOM | 5 | C14 | UDP | 18 | 3.156 | −4.926 | −2.200 | −0.38 | +0.09 | +0.113 | 5.907 |
| ATOM | 6 | N10 | UDP | 18 | 1.143 | −4.585 | 0.739 | −0.06 | +0.01 | +0.039 | 5.907 |
| ATOM | 7 | C11 | UDP | 18 | 1.116 | −3.879 | 1.910 | −0.09 | −0.01 | −0.073 | 5.907 |
| ATOM | 8 | O17 | UDP | 18 | 2.130 | −3.355 | 2.376 | +0.20 | +0.00 | −0.026 | 5.907 |
| ATOM | 9 | N12 | UDP | 18 | −0.104 | −3.772 | 2.530 | −0.04 | +0.02 | −0.292 | 5.907 |
| ATOM | 10 | H12 | UDP | 18 | −0.137 | −3.197 | 3.455 | +0.11 | −0.06 | +0.393 | 5.907 |
| ATOM | 11 | C9 | UDP | 18 | −1.304 | −4.329 | 2.089 | −0.12 | +0.00 | −0.023 | 5.907 |
| ATOM | 12 | O18 | UDP | 18 | −2.317 | −4.133 | 2.762 | +0.13 | +0.01 | −0.026 | 5.907 |
| ATOM | 13 | C7 | UDP | 18 | −1.209 | −5.060 | 0.865 | −0.09 | +0.00 | +0.000 | 5.907 |
| ATOM | 14 | C5 | UDP | 18 | −0.020 | −5.175 | 0.253 | −0.10 | −0.01 | −0.050 | 5.907 |
| ATOM | 15 | O21 | UDP | 18 | 3.652 | −6.560 | 0.982 | −0.02 | −0.32 | −0.537 | 5.907 |
| ATOM | 16 | H21 | UDP | 18 | 3.018 | −6.768 | 1.766 | +0.08 | +0.16 | +0.424 | 5.907 |
| ATOM | 17 | O23 | UDP | 18 | 5.090 | −5.551 | −0.888 | −0.39 | −0.97 | −0.537 | 5.907 |
| ATOM | 18 | H23 | UDP | 18 | 5.674 | −6.354 | −0.621 | +0.08 | +0.80 | +0.424 | 5.907 |
| ATOM | 19 | C25 | UDP | 18 | 2.458 | −5.464 | −3.450 | −0.35 | +0.07 | +0.113 | 5.907 |
| ATOM | 20 | O28 | UDP | 18 | 3.385 | −5.413 | −4.518 | +0.03 | −0.32 | −0.368 | 5.907 |
| ATOM | 21 | P39 | UDP | 18 | 4.037 | −4.229 | −5.334 | −0.55 | +1.52 | +1.210 | 5.907 |
| ATOM | 22 | O30 | UDP | 18 | 5.316 | −3.865 | −4.660 | −0.22 | −1.49 | −0.850 | 5.907 |
| ATOM | 23 | O31 | UDP | 18 | 4.082 | −4.613 | −6.778 | −0.04 | −1.20 | −0.850 | 5.907 |
| ATOM | 24 | O32 | UDP | 18 | 2.913 | −3.158 | −4.973 | +0.05 | −0.52 | −0.510 | 5.907 |
| ATOM | 25 | P33 | UDP | 18 | 2.593 | −1.687 | −5.512 | −0.65 | +1.54 | +1.210 | 5.907 |
| ATOM | 26 | O36 | UDP | 18 | 3.929 | −1.046 | −5.394 | −0.16 | −1.05 | −0.850 | 5.907 |
| ATOM | 27 | O35 | UDP | 18 | 1.401 | −1.051 | −4.893 | −0.76 | −0.49 | −0.255 | 5.907 |
| ATOM | 28 | O34 | UDP | 18 | 2.168 | −1.895 | −7.057 | −0.12 | −1.09 | 0.850 | 5.907 |
| TER | | | | | | | | | | | |

ENDMDL

TABLE 16

| USER | Run = 1 | | | | | | | | | | |
|---|---|---|---|---|---|---|---|---|---|---|---|
| USER | Cluster Rank = 1 | | | | | | | | | | |
| USER | Number of conformations in this cluster = 4 | | | | | | | | | | |
| USER | | | | | | | | | | | |
| USER | RMSD from reference structure = 1.369 A | | | | | | | | | | |
| USER | | | | | | | | | | | |
| USER | Estimated Free Energy of Binding = −10.40 kcal/mol [= (1) + (3)] | | | | | | | | | | |
| USER | Estimated Inhibition Constant, Ki = +2.38e−08 [Temperature = 298.15 K] | | | | | | | | | | |
| USER | | | | | | | | | | | |
| USER | Final Docked Energy = −13.24 kcal/mol [= (1) + (2)] | | | | | | | | | | |
| USER | | | | | | | | | | | |
| USER | (1) Final Intermolecular Energy = −12.58 kcal/mol | | | | | | | | | | |
| USER | (2) Final Internal Energy of Ligand = −0.66 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tranO −1.632364 19.512117 9.441649 | | | | | | | | | | |
| USER | NEWDPF quatO −0.465894 0.713117 0.523838 −10.780137 | | | | | | | | | | |
| USER | NEWDPF ndihe 7 | | | | | | | | | | |
| USER | NEWDPF dihe0 179.95 136.12 179.58 35.98 90.64 41.18 9.31 | | | | | | | | | | |
| USER | | | | | | | | | | | |
| USER | | | | Rank | x | y | z | vdW | Elec | q | RMS |
| ATOM | 1 | N1 | UD1 | 1 | −1.824 | 19.845 | 7.132 | −0.42 | −0.29 | −0.211 | 1.369 |
| ATOM | 2 | C8 | UD1 | 1 | −2.934 | 20.410 | 6.502 | −0.78 | +0.43 | +0.396 | 1.369 |
| ATOM | 3 | N2 | UD1 | 1 | −2.631 | 21.275 | 5.477 | −0.46 | −0.28 | −0.440 | 1.369 |
| ATOM | 4 | H2 | UD1 | 1 | −3.431 | 21.710 | 4.995 | −0.31 | −0.09 | −0.440 | 1.369 |
| ATOM | 5 | C9 | UD1 | 1 | −1.361 | 21.630 | 5.017 | −0.64 | +0.33 | +0.396 | 1.369 |
| ATOM | 6 | C1O | UD1 | 1 | −0.270 | 21.012 | 5.713 | −0.60 | +0.00 | +0.000 | 1.369 |
| ATOM | 7 | C11 | UD1 | 1 | −0.530 | 20.159 | 6.724 | −0.61 | +0.00 | +0.000 | 1.369 |
| ATOM | 8 | O6 | UD1 | 1 | −4.093 | 20.165 | 6.828 | −0.32 | −0.45 | −0.396 | 1.369 |
| ATOM | 9 | O7 | UD1 | 1 | −1.253 | 22.422 | 4.077 | +0.00 | −0.25 | −0.396 | 1.369 |
| ATOM | 10 | C12 | UD1 | 1 | −2.066 | 18.909 | 8.242 | −0.75 | +0.50 | +0.324 | 1.369 |
| ATOM | 11 | C13 | UD1 | 1 | −1.315 | 17.578 | 8.148 | −0.81 | +0.14 | +0.113 | 1.369 |
| ATOM | 12 | C14 | UD1 | 1 | −1.263 | 17.155 | 9.615 | −0.83 | +0.15 | +0.113 | 1.369 |
| ATOM | 13 | C15 | UD1 | 1 | −1.262 | 18.479 | 10.389 | −0.69 | +0.19 | +0.113 | 1.369 |
| ATOM | 14 | O9 | UD1 | 1 | −1.632 | 19.512 | 9.442 | −0.17 | −0.44 | −0.227 | 1.369 |
| ATOM | 15 | O8 | UD1 | 1 | −1.974 | 16.669 | 7.289 | −0.21 | −0.44 | −0.537 | 1.369 |
| ATOM | 16 | H8 | UD1 | 1 | −1.516 | 15.748 | 7.347 | −0.27 | +0.11 | +0.424 | 1.369 |
| ATOM | 17 | O10 | UD1 | 1 | −2.344 | 16.313 | 10.033 | −0.15 | −0.55 | −0.537 | 1.369 |
| ATOM | 18 | HO10 | UD1 | 1 | −2.424 | 15.513 | 9.389 | −0.18 | +0.11 | +0.424 | 1.369 |
| ATOM | 19 | C16 | UD1 | 1 | 0.092 | 18.847 | 10.951 | −0.61 | +0.22 | +0.113 | 1.369 |
| ATOM | 20 | O11 | UD1 | 1 | 0.209 | 18.367 | 12.296 | −0.28 | −0.97 | −0.368 | 1.369 |
| ATOM | 21 | PA | UD1 | 1 | −0.060 | 19.320 | 13.554 | −0.50 | +2.45 | +1.019 | 1.369 |
| ATOM | 22 | O1A | UD1 | 1 | −0.058 | 18.406 | 14.718 | −0.41 | −0.71 | −0.255 | 1.369 |
| ATOM | 23 | 02A | UD1 | 1 | −1.384 | 19.973 | 13.396 | −0.09 | −0.46 | −0.255 | 1.369 |
| ATOM | 24 | 03A | UD1 | 1 | 1.061 | 20.319 | 13.595 | −0.09 | −1.14 | −0.510 | 1.369 |
| ATOM | 25 | PB | UD1 | 1 | 2.633 | 20.205 | 13.643 | −0.59 | +2.49 | +1.019 | 1.369 |
| ATOM | 26 | O1B | UD1 | 1 | 3.087 | 19.447 | 14.832 | −0.17 | −0.66 | −0.255 | 1.369 |
| ATOM | 27 | O2B | UD1 | 1 | 3.280 | 21.537 | 13.510 | −0.17 | −0.47 | −0.255 | 1.369 |
| ATOM | 28 | O | UD1 | 1 | 3.064 | 19.465 | 12.291 | −0.59 | −0.81 | −0.255 | 1.369 |
| TER | | | | | | | | | | | |
| ENDMDL | | | | | | | | | | | |
| MODEL | 4 | | | | | | | | | | |
| USER | Run = 4 | | | | | | | | | | |
| USER | Cluster Rank = 1 | | | | | | | | | | |
| USER | Number of conformations in this cluster = 4 | | | | | | | | | | |
| USER | | | | | | | | | | | |
| USER | RMSD from reference structure = 1.337 A | | | | | | | | | | |
| USER | | | | | | | | | | | |
| USER | Estimated Free Energy of Binding = −10.09 kcal/mol [= (1) + (3)] | | | | | | | | | | |
| USER | Estimated Inhibition Constant, Ki = +4.03e−08 [Temperature = 298.15 K] | | | | | | | | | | |
| USER | | | | | | | | | | | |
| USER | Final Docked Energy = −13.00 kcal/mol [= (1) + (2)] | | | | | | | | | | |
| USER | | | | | | | | | | | |
| USER | (1) Final Intermolecular Energy = −12.27 kcal/mol | | | | | | | | | | |
| USER | (2) Final Internal Energy of Ligand = −0.74 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 −1.265923 19.405158 9.226950 | | | | | | | | | | |
| USER | NEWDPF quat0 −0.608979 0.494057 0.620526 −12.489947 | | | | | | | | | | |
| USER | NEWDPF ndihe 7 | | | | | | | | | | |
| USER | NEWDPF dihe0 −105.04 135.16 −48.92 33.68 −14.24 −0.03 −21.94 | | | | | | | | | | |
| USER | | | | | | | | | | | |
| USER | | | | Rank | x | y | z | vdW | Elec | q | RMS |
| ATOM | 1 | N1 | UD1 | 1 | −1.504 | 19.857 | 6.942 | −0.39 | −0.28 | −0.211 | 1.337 |
| ATOM | 2 | C8 | UD1 | 1 | −2.609 | 20.487 | 6.369 | −0.73 | +0.43 | +0.396 | 1.337 |
| ATOM | 3 | N2 | UD1 | 1 | −2.304 | 21.391 | 5.380 | −0.45 | −0.30 | −0.440 | 1.337 |
| ATOM | 4 | H2 | UD1 | 1 | −3.101 | 21.874 | 4.940 | −0.29 | −0.03 | +0.440 | 1.337 |
| ATOM | 5 | C9 | UD1 | 1 | −1.034 | 21.728 | 4.904 | −0.62 | +0.36 | +0.396 | 1.337 |
| ATOM | 6 | C10 | UD1 | 1 | 0.053 | 21.042 | 5.541 | −0.59 | +0.00 | +0.000 | 1.337 |
| ATOM | 7 | C11 | UD1 | 1 | −0.211 | 20.149 | 6.515 | −0.61 | +0.00 | +0.000 | 1.337 |
| ATOM | 8 | O6 | UD1 | 1 | −3.768 | 20.264 | 6.714 | −0.31 | −0.44 | −0.396 | 1.337 |
| ATOM | 9 | O7 | UD1 | 1 | −0.924 | 22.561 | 4.000 | −0.01 | −0.29 | −0.396 | 1.337 |
| ATOM | 10 | C12 | UD1 | 1 | −1.749 | 18.876 | 8.011 | −0.74 | +0.47 | +0.324 | 1.337 |
| ATOM | 11 | C13 | UD1 | 1 | −1.044 | 17.528 | 7.834 | −0.68 | +0.14 | +0.113 | 1.337 |
| ATOM | 12 | C14 | UD1 | 1 | −0.971 | 17.032 | 9.277 | −0.84 | +0.15 | +0.113 | 1.337 |
| ATOM | 13 | C15 | UD1 | 1 | −0.906 | 18.316 | 10.113 | −0.67 | +0.19 | +0.113 | 1.337 |
| ATOM | 14 | O9 | UD1 | 1 | −1.266 | 19.405 | 9.227 | −0.09 | −0.40 | −0.227 | 1.337 |
| ATOM | 15 | O8 | UD1 | 1 | −1.754 | 16.683 | 6.950 | −0.06 | −0.49 | −0.537 | 1.337 |
| ATOM | 16 | H8 | UD1 | 1 | −2.338 | 16.030 | 7.492 | −0.15 | +0.19 | +0.424 | 1.337 |
| ATOM | 17 | O10 | UD1 | 1 | −2.068 | 16.205 | 9.681 | −0.20 | −0.49 | −0.537 | 1.337 |
| ATOM | 18 | HO10 | UD1 | 1 | −2.201 | 15.450 | 8.994 | −0.30 | +0.03 | +0.424 | 1.337 |
| ATOM | 19 | C16 | UD1 | 1 | 0.472 | 18.612 | 10.656 | −0.64 | +0.23 | +0.113 | 1.337 |
| ATOM | 20 | O11 | UD1 | 1 | 0.415 | 18.755 | 12.082 | −0.37 | −0.93 | −0.368 | 1.337 |
| ATOM | 21 | PA | UD1 | 1 | 1.243 | 19.893 | 12.845 | −0.41 | +2.62 | +1.019 | 1.337 |
| ATOM | 22 | O1A | UD1 | 1 | 0.633 | 21.162 | 12.390 | −0.03 | −0.45 | −0.255 | 1.337 |
| ATOM | 23 | O2A | UD1 | 1 | 2.668 | 19.815 | 12.436 | −0.53 | −0.73 | −0.255 | 1.337 |
| ATOM | 24 | O3A | UD1 | 1 | 1.064 | 19.654 | 14.317 | −0.28 | −1.36 | −0.510 | 1.337 |
| ATOM | 25 | PB | UD1 | 1 | −0.188 | 19.563 | 15.270 | −0.57 | +2.24 | +1.019 | 1.337 |
| ATOM | 26 | O1B | UD1 | 1 | −0.942 | 18.305 | 15.061 | −0.40 | −0.67 | −0.255 | 1.337 |
| ATOM | 27 | O2B | UD1 | 1 | −1.036 | 20.782 | 15.174 | −0.05 | −0.46 | −0.255 | 1.337 |
| ATOM | 28 | O | UD1 | 1 | 0.385 | 19.648 | 16.762 | −0.30 | −0.67 | −0.255 | 1.337 |
| TER | | | | | | | | | | | |
| ENDMDL | | | | | | | | | | | |
| MODEL | 6 | | | | | | | | | | |
| USER | Run = 6 | | | | | | | | | | |
| USER | Cluster Rank = 1 | | | | | | | | | | |
| USER | Number of conformations in this cluster = 4 | | | | | | | | | | |
| USER | | | | | | | | | | | |
| USER | RMSD from reference structure = 1.224 A | | | | | | | | | | |
| USER | | | | | | | | | | | |
| USER | Estimated Free Energy of Binding = −9.83 kcal/mol [= (1) + (3)] | | | | | | | | | | |
| USER | Estimated Inhibition Constant, Ki = +6.19e−08 [Temperature = 298.15 K] | | | | | | | | | | |
| USER | | | | | | | | | | | |
| USER | Final Docked Energy = −12.51 kcal/mol [= (1) + (2)] | | | | | | | | | | |
| USER | | | | | | | | | | | |
| USER | (1) Final Intermolecular Energy = −12.01 kcal/mol | | | | | | | | | | |
| USER | (2) Final Internal Energy of Ligand = −0.50 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 −1.501670 19.962002 9.532995 | | | | | | | | | | |
| USER | NEWDPF quat0 0.323925 0.675547 0.662351 −9.134749 | | | | | | | | | | |
| USER | NEWDPF ndihe 7 | | | | | | | | | | |
| USER | NEWDPF dihe0 −176.62 96.89 −48.25 81.53 −50.86 −6.24 7.25 | | | | | | | | | | |
| USER | | | | | | | | | | | |
| USER | | | | Rank | x | y | z | vdW | Elec | q | RMS |
| ATOM | 1 | N1 | UD1 | 1 | −1.769 | 19.981 | 7.207 | −0.41 | −0.29 | −0.211 | 1.224 |
| ATOM | 2 | C8 | UD1 | 1 | −2.892 | 20.474 | 6.542 | −0.80 | +0.44 | +0.396 | 1.224 |
| ATOM | 3 | N2 | UD1 | 1 | −2.614 | 21.185 | 5.399 | −0.43 | −0.28 | −0.440 | 1.224 |
| ATOM | 4 | H2 | UD1 | 1 | −3.425 | 21.564 | 4.888 | −0.33 | −0.11 | +0.440 | 1.224 |
| ATOM | 5 | C9 | UD1 | 1 | −1.356 | 21.452 | 4.854 | −0.55 | +0.34 | +0.396 | 1.224 |
| ATOM | 6 | C10 | UD1 | 1 | −0.249 | 20.916 | 5.592 | −0.60 | +0.00 | +0.000 | 1.224 |
| ATOM | 7 | C11 | UD1 | 1 | −0.486 | 20.213 | 6.718 | −0.61 | +0.00 | +0.000 | 1.224 |
| ATOM | 8 | O6 | UD1 | 1 | −4.043 | 20.296 | 6.936 | −0.24 | −0.47 | −0.396 | 1.224 |
| ATOM | 9 | O7 | UD1 | 1 | −1.271 | 22.107 | 3.811 | −0.09 | −0.24 | −0.396 | 1.224 |
| ATOM | 10 | C12 | UD1 | 1 | −1.984 | 19.209 | 8.442 | −0.71 | +0.53 | +0.324 | 1.224 |
| ATOM | 11 | C13 | UD1 | 1 | −1.254 | 17.865 | 8.506 | −0.77 | +0.15 | +0.113 | 1.224 |
| ATOM | 12 | C14 | UD1 | 1 | −1.157 | 17.645 | 10.015 | −0.75 | +0.17 | +0.113 | 1.224 |
| ATOM | 13 | C15 | UD1 | 1 | −1.112 | 19.062 | 10.600 | −0.64 | +0.20 | +0.113 | 1.224 |
| ATOM | 14 | O9 | UD1 | 1 | −1.502 | 19.962 | 9.533 | −0.25 | −0.45 | −0.227 | 1.224 |
| ATOM | 15 | O8 | UD1 | 1 | −1.953 | 16.858 | 7.802 | −0.33 | −0.49 | −0.537 | 1.224 |
| ATOM | 16 | H8 | UD1 | 1 | −1.541 | 15.939 | 8.016 | −0.18 | +0.19 | +0.424 | 1.224 |
| ATOM | 17 | O10 | UD1 | 1 | −2.233 | 16.886 | 10.579 | −0.25 | −0.70 | −0.537 | 1.224 |
| ATOM | 18 | HO10 | UD1 | 1 | −2.773 | 16.442 | 9.823 | +0.00 | +0.42 | +0.424 | 1.224 |
| ATOM | 19 | C16 | UD1 | 1 | 0.265 | 19.479 | 11.062 | −0.59 | +0.22 | +0.113 | 1.224 |
| ATOM | 20 | O11 | UD1 | 1 | 0.405 | 19.229 | 12.467 | −0.33 | −0.92 | −0.368 | 1.224 |
| ATOM | 21 | PA | UD1 | 1 | 1.265 | 20.197 | 13.410 | −0.48 | +2.41 | +1.019 | 1.224 |
| ATOM | 22 | O1A | UD1 | 1 | 1.157 | 21.527 | 12.770 | −0.04 | −0.45 | −0.255 | 1.224 |
| ATOM | 23 | O2A | UD1 | 1 | 2.674 | 19.732 | 13.429 | −0.44 | −0.74 | −0.255 | 1.224 |
| ATOM | 24 | O3A | UD1 | 1 | 0.636 | 20.151 | 14.773 | +0.01 | −1.10 | −0.510 | 1.224 |
| ATOM | 25 | PB | UD1 | 1 | −0.705 | 19.552 | 15.345 | −0.63 | +2.15 | +1.019 | 1.224 |
| ATOM | 26 | O1B | UD1 | 1 | −0.649 | 18.074 | 15.428 | −0.46 | −0.68 | −0.255 | 1.224 |
| ATOM | 27 | O2B | UD1 | 1 | −1.889 | 20.056 | 14.599 | −0.09 | −0.44 | −0.255 | 1.224 |
| ATOM | 28 | O | UD1 | 1 | −0.898 | 20.203 | 16.794 | −0.25 | −0.64 | −0.255 | 1.224 |
| TER | | | | | | | | | | | |
| ENDMDL | | | | | | | | | | | |
| MODEL | 9 | | | | | | | | | | |
| USER | Run = 9 | | | | | | | | | | |
| USER | Cluster Rank = 1 | | | | | | | | | | |
| USER | Number of conformations in this cluster = 4 | | | | | | | | | | |
| USER | | | | | | | | | | | |
| USER | RMSD from reference structure = 1.336 A | | | | | | | | | | |
| USER | | | | | | | | | | | |
| USER | Estimated Free Energy of Binding = −9.64 kcal/mol [= (1) + (3)] | | | | | | | | | | |
| USER | Estimated Inhibition Constant, Ki = +8.57e−08 [Temperature = 298.15 K] | | | | | | | | | | |
| USER | | | | | | | | | | | |
| USER | Final Docked Energy = −12.51 kcal/mol [= (1) + (2)] | | | | | | | | | | |
| USER | | | | | | | | | | | |
| USER | (1) Final Intermolecular Energy = −11.82 kcal/mol | | | | | | | | | | |
| USER | (2) Final Internal Energy of Ligand = −0.69 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 −1.801914 19.676305 9.934226 | | | | | | | | | | |
| USER | NEWDPF quat0 0.278019 0.743884 0.607736 −15.232066 | | | | | | | | | | |
| USER | NEWDPF ndihe 7 | | | | | | | | | | |
| USER | NEWDPF dihe0 −174.00 109.35 87.54 151.81 −135.47 −94.79 −43.87 | | | | | | | | | | |
| USER | | | | | | | | | | | |
| USER | | | | Rank | x | y | z | vdW | Elec | q | RMS |
| ATOM | 1 | N1 | UD1 | 1 | −1.859 | 19.648 | 7.594 | −0.43 | −0.31 | −0.211 | 1.336 |
| ATOM | 2 | C8 | UD1 | 1 | −2.889 | 20.183 | 6.819 | −0.84 | +0.48 | +0.396 | 1.336 |
| ATOM | 3 | N2 | UD1 | 1 | −2.471 | 20.848 | 5.691 | −0.36 | −0.38 | −0.440 | 1.336 |
| ATOM | 4 | H2 | UD1 | 1 | −3.210 | 21.256 | 5.101 | −0.20 | +0.09 | +0.440 | 1.336 |
| ATOM | 5 | C9 | UD1 | 1 | −1.155 | 21.032 | 5.257 | −0.54 | +0.42 | +0.396 | 1.336 |
| ATOM | 6 | C10 | UD1 | 1 | −0.151 | 20.457 | 6.104 | −0.61 | +0.00 | +0.000 | 1.336 |
| ATOM | 7 | C11 | UD1 | 1 | −0.526 | 19.798 | 7.218 | −0.61 | +0.00 | +0.000 | 1.336 |
| ATOM | 8 | O6 | UD1 | 1 | −4.078 | 20.077 | 7.111 | −0.20 | −0.51 | −0.396 | 1.336 |
| ATOM | 9 | O7 | UD1 | 1 | −0.941 | 21.654 | 4.212 | −0.10 | −0.34 | −0.396 | 1.336 |
| ATOM | 10 | C12 | UD1 | 1 | −2.226 | 18.922 | 8.820 | −0.62 | +0.56 | +0.324 | 1.336 |
| ATOM | 11 | C13 | UD1 | 1 | −1.581 | 17.542 | 8.980 | −0.83 | +0.15 | +0.113 | 1.336 |
| ATOM | 12 | C14 | UD1 | 1 | −1.632 | 17.358 | 10.495 | −0.78 | +0.16 | +0.113 | 1.336 |
| ATOM | 13 | C15 | UD1 | 1 | −1.560 | 18.785 | 11.051 | −0.71 | +0.20 | +0.113 | 1.336 |
| ATOM | 14 | O9 | UD1 | 1 | −1.802 | 19.676 | 9.934 | −0.24 | −0.49 | −0.227 | 1.336 |
| ATOM | 15 | O8 | UD1 | 1 | −2.270 | 16.556 | 8.236 | −0.29 | −0.40 | −0.537 | 1.336 |
| ATOM | 16 | H8 | UD1 | 1 | −1.962 | 15.621 | 8.538 | −0.34 | +0.05 | +0.424 | 1.336 |
| ATOM | 17 | O10 | UD1 | 1 | −2.795 | 16.672 | 10.975 | −0.17 | −0.70 | −0.537 | 1.336 |
| ATOM | 18 | HO10 | UD1 | 1 | −3.171 | 16.070 | 10.230 | −0.06 | +0.40 | +0.424 | 1.336 |
| ATOM | 19 | C16 | UD1 | 1 | −0.209 | 19.140 | 11.627 | −0.59 | +0.23 | +0.113 | 1.336 |
| ATOM | 20 | O11 | UD1 | 1 | −0.371 | 20.030 | 12.739 | −0.05 | −0.72 | −0.368 | 1.336 |
| ATOM | 21 | PA | UD1 | 1 | 0.271 | 19.709 | 14.171 | −0.48 | +2.37 | +1.019 | 1.336 |
| ATOM | 22 | O1A | UD1 | 1 | −0.252 | 18.370 | 14.519 | −0.34 | −0.73 | −0.255 | 1.336 |
| ATOM | 23 | O2A | UD1 | 1 | −0.188 | 20.727 | 15.149 | +0.02 | −0.49 | −0.255 | 1.336 |
| ATOM | 24 | O3A | UD1 | 1 | 1.763 | 19.726 | 13.996 | −0.33 | −1.48 | −0.510 | 1.336 |
| ATOM | 25 | PB | UD1 | 1 | 2.803 | 20.819 | 13.536 | −0.64 | +2.16 | +1.019 | 1.336 |
| ATOM | 26 | O1B | UD1 | 1 | 3.984 | 20.205 | 12.886 | −0.55 | −0.71 | −0.255 | 1.336 |
| ATOM | 27 | O2B | UD1 | 1 | 3.154 | 21.740 | 14.650 | −0.02 | −0.44 | −0.255 | 1.336 |
| ATOM | 28 | O | UD1 | 1 | 2.018 | 21.775 | 12.520 | −0.07 | −0.45 | −0.255 | 1.336 |
| TER | | | | | | | | | | | |
| ENDMDL | | | | | | | | | | | |
| MODEL | 3 | | | | | | | | | | |
| USER | Run = 3 | | | | | | | | | | |
| USER | Cluster Rank = 2 | | | | | | | | | | |
| USER | Number of conformations in this cluster = 1 | | | | | | | | | | |
| USER | | | | | | | | | | | |
| USER | RMSD from reference structure = 1.112 A | | | | | | | | | | |
| USER | | | | | | | | | | | |
| USER | Estimated Free Energy of Binding = −9.92 kcal/mol [= (1) + (3)] | | | | | | | | | | |
| USER | Estimated Inhibition Constant, Ki = +5.31e−08 [Temperature = 298.15 K] | | | | | | | | | | |
| USER | | | | | | | | | | | |
| USER | Final Docked Energy = −12.49 kcal/mol [= (1) + (2)] | | | | | | | | | | |
| USER | | | | | | | | | | | |
| USER | (1) Final Intermolecular Energy = −12.10 kcal/mol | | | | | | | | | | |
| USER | (2) Final Internal Energy of Ligand −0.39 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDFF move m_udp.pdbq | | | | | | | | | | |
| USER | NEWDPF about −1.182000 20.257999 9.670000 | | | | | | | | | | |
| USER | NEWDPF tran0 −1.497053 19.925561 9.322984 | | | | | | | | | | |
| USER | NEWDPF quat0 0.015430 0.595437 0.803254 −12.063578 | | | | | | | | | | |
| USER | NEWDPF ndihe 7 | | | | | | | | | | |
| USER | NEWDPF dihe0 −156.03 −180.00 −96.01 −166.78 37.73 −66.89 −57.92 | | | | | | | | | | |
| USER | | | | | | | | | | | |
| USER | | | | Rank | x | y | z | vdW | Elec | q | RMS |
| ATOM | 1 | N1 | UD1 | 2 | −1.708 | 20.076 | 6.996 | −0.41 | −0.28 | −0.211 | 1.112 |
| ATOM | 2 | C8 | UD1 | 2 | −2.784 | 20.669 | 6.335 | −0.81 | +0.40 | +0.396 | 1.112 |
| ATOM | 3 | N2 | UD1 | 2 | −2.438 | 21.419 | 5.236 | −0.37 | −0.25 | −0.440 | 1.112 |
| ATOM | 4 | H2 | UD1 | 2 | −3.212 | 21.871 | 4.728 | −0.29 | −0.18 | +0.440 | 1.112 |
| ATOM | 5 | C9 | UD1 | 2 | −1.153 | 21.636 | 4.730 | −0.61 | +0.34 | +0.396 | 1.112 |
| ATOM | 6 | C10 | UD1 | 2 | −0.098 | 20.997 | 5.463 | −0.60 | +0.00 | +0.000 | 1.112 |
| ATOM | 7 | C11 | UD1 | 2 | −0.402 | 20.255 | 6.546 | −0.61 | +0.00 | +0.000 | 1.112 |
| ATOM | 8 | O6 | UD1 | 2 | −3.952 | 20.542 | 6.695 | −0.30 | −0.43 | −0.396 | 1.112 |
| ATOM | 9 | O7 | UD1 | 2 | −1.005 | 22.335 | 3.724 | −0.05 | −0.27 | −0.396 | 1.112 |
| ATOM | 10 | C12 | UD1 | 2 | −1.998 | 19.259 | 8.185 | −0.73 | +0.51 | +0.324 | 1.112 |
| ATOM | 11 | C13 | UD1 | 2 | −1.351 | 17.871 | 8.196 | −0.79 | +0.15 | +0.113 | 1.112 |
| ATOM | 12 | C14 | UD1 | 2 | −1.303 | 17.571 | 9.692 | −0.79 | +0.16 | +0.113 | 1.112 |
| ATOM | 13 | C15 | UD1 | 2 | −1.187 | 18.951 | 10.350 | −0.65 | +0.20 | +0.113 | 1.112 |
| ATOM | 14 | O9 | UD1 | 2 | −1.497 | 19.926 | 9.323 | −0.21 | −0.43 | −0.227 | 1.112 |
| ATOM | 15 | O8 | UD1 | 2 | −2.092 | 16.945 | 7.426 | −0.24 | −0.49 | −0.537 | 1.112 |
| ATOM | 16 | H8 | UD1 | 2 | −1.998 | 16.004 | 7.835 | −0.20 | +0.17 | +0.424 | 1.112 |
| ATOM | 17 | O10 | UD1 | 2 | −2.435 | 16.851 | 10.195 | −0.26 | −0.67 | −0.537 | 1.112 |
| ATOM | 18 | HO10 | UD1 | 2 | −2.217 | 15.845 | 10.225 | +0.01 | +0.10 | +0.424 | 1.112 |
| ATOM | 19 | C16 | UD1 | 2 | 0.201 | 19.260 | 10.861 | −0.59 | +0.22 | +0.113 | 1.112 |
| ATOM | 20 | O11 | UD1 | 2 | 0.141 | 20.313 | 11.833 | −0.03 | −0.70 | −0.368 | 1.112 |
| ATOM | 21 | PA | UD1 | 2 | 1.255 | 20.467 | 12.973 | −0.54 | +2.22 | +1.019 | 1.112 |
| ATOM | 22 | O1A | UD1 | 2 | 1.870 | 21.787 | 12.712 | −0.09 | −0.44 | −0.255 | 1.112 |
| ATOM | 23 | O2A | UD1 | 2 | 2.254 | 19.379 | 12.828 | −0.42 | −0.88 | −0.255 | 1.112 |
| ATOM | 24 | O3A | UD1 | 2 | 0.541 | 20.405 | 14.293 | +0.01 | −1.05 | −0.510 | 1.112 |
| ATOM | 25 | PB | UD1 | 2 | 0.353 | 19.288 | 15.389 | −0.55 | +2.42 | +1.019 | 1.112 |
| ATOM | 26 | O1B | UD1 | 2 | 1.461 | 18.305 | 15.360 | −0.36 | −0.80 | −0.255 | 1.112 |
| ATOM | 27 | O2B | UD1 | 2 | −0.994 | 18.662 | 15.304 | −0.38 | −0.60 | −0.255 | 1.112 |
| ATOM | 28 | O | UD1 | 2 | 0.299 | 20.051 | 16.795 | −0.32 | −0.67 | −0.255 | 1.112 |
| TER | | | | | | | | | | | |
| ENDMDL | | | | | | | | | | | |
| MODEL | 7 | | | | | | | | | | |
| USER | Run = 7 | | | | | | | | | | |
| USER | Cluster Rank = 3 | | | | | | | | | | |
| USER | Number of conformations in this cluster = 2 | | | | | | | | | | |
| USER | | | | | | | | | | | |
| USER | RMSD from reference structure = 7.924 A | | | | | | | | | | |
| USER | | | | | | | | | | | |
| USER | Estimated Free Energy of Binding = −8.22 kcal/mol [= (1) + (3)] | | | | | | | | | | |
| USER | Estimated Inhibition Constant, Ki = +9.39e−07 [Temperature = 298.15 K] | | | | | | | | | | |
| USER | | | | | | | | | | | |
| USER | Final Docked Energy = −11.47 kcal/mol [= (1) + (2)] | | | | | | | | | | |
| USER | | | | | | | | | | | |
| USER | (1) Final Intermolecular Energy = −10.40 kcal/mol | | | | | | | | | | |
| USER | (2) Final Internal Energy of Ligand = −1.06 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 4.351957 15.649956 19.507160 | | | | | | | | | | |
| USER | NEWDPF quat0 −0.493337 −0.186735 −0.849558 −60.910644 | | | | | | | | | | |
| USER | NEWDPF ndihe 7 | | | | | | | | | | |
| USER | NEWDPF dihe0 133.56 70.58 13.73 −1.17 −119.13 17.14 −53.91 | | | | | | | | | | |
| USER | | | | | | | | | | | |
| USER | | | | Rank | x | y | z | vdW | Elec | q | RMS |
| ATOM | 1 | N1 | UD1 | 3 | 3.103 | 16.083 | 17.575 | −0.33 | −0.37 | −0.211 | 7.924 |
| ATOM | 2 | C8 | UD1 | 3 | 1.877 | 15.490 | 17.274 | −0.83 | +0.67 | +0.396 | 7.924 |
| ATOM | 3 | N2 | UD1 | 3 | 0.950 | 16.340 | 16.720 | −0.45 | −0.83 | −0.440 | 7.924 |
| ATOM | 4 | H2 | UD1 | 3 | 0.033 | 15.930 | 16.489 | −0.03 | +0.72 | +0.440 | 7.924 |
| ATOM | 5 | C9 | UD1 | 3 | 1.112 | 17.699 | 16.435 | −0.65 | +0.95 | +0.396 | 7.924 |
| ATOM | 6 | C10 | UD1 | 3 | 2.396 | 18.239 | 16.773 | −0.43 | +0.00 | +0.000 | 7.924 |
| ATOM | 7 | C11 | UD1 | 3 | 3.329 | 17.434 | 17.318 | −0.42 | +0.00 | +0.000 | 7.924 |
| ATOM | 8 | O6 | UD1 | 3 | 1.630 | 14.304 | 17.484 | −0.21 | −0.61 | −0.396 | 7.924 |
| ATOM | 9 | O7 | UD1 | 3 | 0.179 | 18.330 | 15.929 | −0.29 | −0.98 | −0.396 | 7.924 |
| ATOM | 10 | C12 | UD1 | 3 | 4.148 | 15.237 | 18.173 | −0.57 | +0.50 | +0.324 | 7.924 |
| ATOM | 11 | C13 | UD1 | 3 | 5.519 | 15.319 | 17.497 | −0.62 | +0.16 | +0.113 | 7.924 |
| ATOM | 12 | C14 | UD1 | 3 | 6.444 | 14.888 | 18.634 | −0.65 | +0.17 | +0.113 | 7.924 |
| ATOM | 13 | C15 | UD1 | 3 | 5.708 | 15.328 | 19.905 | −0.43 | +0.17 | +0.113 | 7.924 |
| ATOM | 14 | O9 | UD1 | 3 | 4.352 | 15.650 | 19.507 | +0.02 | −0.34 | −0.227 | 7.924 |
| ATOM | 15 | O8 | UD1 | 3 | 5.578 | 14.506 | 16.341 | −0.54 | −0.81 | −0.537 | 7.924 |
| ATOM | 16 | H8 | UD1 | 3 | 6.114 | 14.990 | 15.607 | −0.55 | +0.35 | +0.424 | 7.924 |
| ATOM | 17 | O10 | UD1 | 3 | 6.740 | 13.486 | 18.665 | −0.20 | −0.89 | −0.537 | 7.924 |
| ATOM | 18 | HO10 | UD1 | 3 | 6.092 | 12.984 | 18.043 | +0.04 | +0.76 | +0.424 | 7.924 |
| ATOM | 19 | C16 | UD1 | 3 | 6.296 | 16.563 | 20.545 | −0.46 | +0.17 | +0.113 | 7.924 |
| ATOM | 20 | O11 | UD1 | 3 | 5.958 | 16.601 | 21.937 | −0.03 | −0.54 | −0.368 | 7.924 |
| ATOM | 21 | PA | UD1 | 3 | 5.153 | 17.832 | 22.572 | −0.55 | +1.78 | +1.019 | 7.924 |
| ATOM | 22 | O1A | UD1 | 3 | 5.709 | 19.025 | 21.896 | +0.03 | −0.53 | −0.255 | 7.924 |
| ATOM | 23 | O2A | UD1 | 3 | 5.414 | 17.882 | 24.033 | −0.14 | −0.46 | −0.255 | 7.924 |
| ATOM | 24 | O3A | UD1 | 3 | 3.700 | 17.610 | 22.265 | −0.13 | −0.89 | −0.510 | 7.924 |
| ATOM | 25 | PB | UD1 | 3 | 2.741 | 18.065 | 21.099 | −0.69 | +2.19 | +1.019 | 7.924 |
| ATOM | 26 | O1B | UD1 | 3 | 1.894 | 16.947 | 20.624 | −0.14 | −0.55 | −0.255 | 7.924 |
| ATOM | 27 | O2B | UD1 | 3 | 3.494 | 18.740 | 20.008 | +0.03 | −0.53 | −0.255 | 7.924 |
| ATOM | 28 | O | UD1 | 3 | 1.848 | 19.250 | 21.698 | −0.65 | −0.80 | −0.255 | 7.924 |
| TER | | | | | | | | | | | |
| ENDMDL | | | | | | | | | | | |
| MODEL | 8 | | | | | | | | | | |
| USER | Run = 8 | | | | | | | | | | |
| USER | Cluster Rank = 3 | | | | | | | | | | |
| USER | Number of conformations in this cluster = 2 | | | | | | | | | | |
| USER | | | | | | | | | | | |
| USER | RMSD from reference structure = 7.896 A | | | | | | | | | | |
| USER | | | | | | | | | | | |
| USER | Estimated Free Energy of Binding = −8.11 kcal/mol [= (1) + (3)] | | | | | | | | | | |
| USER | Estimated Inhibition Constant, Ki = +1.13e−06 [Temperature = 298.15 K] | | | | | | | | | | |
| USER | | | | | | | | | | | |
| USER | Final Docked Energy = −11.16 kcal/mol [= (1) + (2)] | | | | | | | | | | |
| USER | | | | | | | | | | | |
| USER | (1) Final Intermolecular Energy = −10.29 kcal/mol | | | | | | | | | | |
| USER | (2) Final Internal Energy of Ligand = −0.87 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 4.600642 15.689170 19.538800 | | | | | | | | | | |
| USER | NEWDPF quat0 0.567262 0.320468 0.758626 54.723788 | | | | | | | | | | |
| USER | NEWDPF ndihe 7 | | | | | | | | | | |
| USER | NEWDPF dihe0 123.18 37.99 −89.31 154.06 −99.82 15.33 −89.05 | | | | | | | | | | |
| USER | | | | | | | | | | | |
| USER | | | | Rank | x | y | z | vdW | Elec | q | RMS |
| ATOM | 1 | N1 | UD1 | 3 | 3.165 | 16.207 | 17.764 | −0.30 | −0.37 | −0.211 | 7.896 |
| ATOM | 2 | C8 | UD1 | 3 | 1.840 | 15.781 | 17.665 | −0.79 | +0.70 | +0.396 | 7.896 |
| ATOM | 3 | N2 | UD1 | 3 | 0.968 | 16.729 | 17.184 | −0.53 | −0.91 | −0.440 | 7.896 |
| ATOM | 4 | H2 | UD1 | 3 | −0.018 | 16.443 | 17.103 | +0.05 | +0.93 | +0.440 | 7.896 |
| ATOM | 5 | C9 | UD1 | 3 | 1.271 | 18.036 | 16.793 | −0.62 | +0.95 | +0.396 | 7.896 |
| ATOM | 6 | C10 | UD1 | 3 | 2.651 | 18.403 | 16.923 | −0.42 | +0.00 | +0.000 | 7.896 |
| ATOM | 7 | C11 | UD1 | 3 | 3.533 | 17.498 | 17.392 | −0.40 | +0.00 | +0.000 | 7.896 |
| ATOM | 8 | O6 | UD1 | 3 | 1.467 | 14.654 | 17.982 | −0.13 | −0.65 | −0.396 | 7.896 |
| ATOM | 9 | O7 | UD1 | 3 | 0.371 | 18.770 | 16.374 | −0.18 | −0.98 | −0.396 | 7.896 |
| ATOM | 10 | C12 | UD1 | 3 | 4.159 | 15.249 | 18.273 | −0.56 | +0.50 | +0.324 | 7.896 |
| ATOM | 11 | C13 | UD1 | 3 | 5.422 | 15.103 | 17.418 | −0.66 | +0.17 | +0.113 | 7.896 |
| ATOM | 12 | C14 | UD1 | 3 | 6.429 | 14.597 | 18.449 | −0.70 | +0.17 | +0.113 | 7.896 |
| ATOM | 13 | C15 | UD1 | 3 | 5.943 | 15.195 | 19.775 | −0.48 | +0.17 | +0.113 | 7.896 |
| ATOM | 14 | O9 | UD1 | 3 | 4.601 | 15.689 | 19.539 | +0.03 | −0.34 | −0.227 | 7.896 |
| ATOM | 15 | O8 | UD1 | 3 | 5.209 | 14.238 | 16.320 | −0.49 | −0.97 | −0.537 | 7.896 |
| ATOM | 16 | H8 | UD1 | 3 | 5.546 | 14.689 | 15.457 | −0.57 | +0.41 | +0.424 | 7.896 |
| ATOM | 17 | O10 | UD1 | 3 | 6.534 | 13.171 | 18.532 | −0.15 | −0.91 | −0.537 | 7.896 |
| ATOM | 18 | HO10 | UD1 | 3 | 5.591 | 12.757 | 18.557 | +0.05 | +0.71 | +0.424 | 7.896 |
| ATOM | 19 | C16 | UD1 | 3 | 6.776 | 16.363 | 20.251 | −0.50 | +0.17 | +0.113 | 7.896 |
| ATOM | 20 | O11 | UD1 | 3 | 6.112 | 17.028 | 21.333 | +0.02 | −0.58 | −0.368 | 7.896 |
| ATOM | 21 | PA | UD1 | 3 | 5.765 | 18.590 | 21.271 | −0.38 | +1.94 | +1.019 | 7.896 |
| ATOM | 22 | O1A | UD1 | 3 | 6.663 | 19.127 | 20.225 | +0.03 | −0.52 | −0.255 | 7.896 |
| ATOM | 23 | O2A | UD1 | 3 | 6.058 | 19.207 | 22.589 | −0.05 | −0.57 | −0.255 | 7.896 |
| ATOM | 24 | O3A | UD1 | 3 | 4.312 | 18.702 | 20.906 | +0.04 | −1.01 | −0.510 | 7.896 |
| ATOM | 25 | PB | UD1 | 3 | 2.968 | 18.251 | 21.597 | −0.73 | +2.12 | +1.019 | 7.896 |
| ATOM | 26 | O1B | UD1 | 3 | 3.073 | 18.297 | 23.074 | −0.23 | −0.47 | −0.255 | 7.896 |
| ATOM | 27 | O2B | UD1 | 3 | 2.505 | 16.935 | 21.079 | −0.19 | −0.47 | −0.255 | 7.896 |
| ATOM | 28 | O | UD1 | 3 | 1.840 | 19.255 | 21.068 | −0.73 | −0.88 | −0.255 | 7.896 |
| TER | | | | | | | | | | | |
| ENDMDL | | | | | | | | | | | |
| MODEL | 10 | | | | | | | | | | |
| USER | Run = 10 | | | | | | | | | | |
| USER | Cluster Rank = 4 | | | | | | | | | | |
| USER | Number of conformations in this cluster = 1 | | | | | | | | | | |
| USER | | | | | | | | | | | |
| USER | RMSD from reference structure = 8.626 A | | | | | | | | | | |
| USER | | | | | | | | | | | |
| USER | Estimated Free Energy of Binding = −7.75 kcal/mol [= (1) + (3)] | | | | | | | | | | |
| USER | Estimated Inhibition Constant, Ki = +2.08e−06 [Temperature = 298.15 K] | | | | | | | | | | |
| USER | | | | | | | | | | | |
| USER | Final Docked Energy = −10.63 kcal/mol [= (1) + (2)] | | | | | | | | | | |
| USER | | | | | | | | | | | |
| USER | (1) Final Intermolecular Energy = −9.93 kcal/mol | | | | | | | | | | |
| USER | (2) Final Internal Energy of Ligand = −0.70 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 4.652540 15.568594 19.549531 | | | | | | | | | | |
| USER | NEWDPF quat0 −0.485208 −0.203504 −0.850388 −61.166312 | | | | | | | | | | |
| USER | NEWDPF ndihe 7 | | | | | | | | | | |
| USER | NEWDPF dihe0 109.54 59.29 −76.53 29.66 71.64 21.75 −54.46 | | | | | | | | | | |
| USER | | | | | | | | | | | |
| USER | | | | Rank | x | y | z | vdW | Elec | q | RMS |
| ATOM | 1 | N1 | UD1 | 4 | 3.377 | 15.966 | 17.627 | −0.31 | −0.36 | −0.211 | 8.626 |
| ATOM | 2 | C8 | UD1 | 4 | 2.153 | 15.358 | 17.348 | −0.81 | +0.66 | +0.396 | 8.626 |
| ATOM | 3 | N2 | UD1 | 4 | 1.211 | 16.192 | 16.795 | −0.50 | −0.82 | −0.440 | 8.626 |
| ATOM | 4 | H2 | UD1 | 4 | 0.296 | 15.771 | 16.580 | +0.02 | +0.69 | +0.440 | 8.626 |
| ATOM | 5 | C9 | UD1 | 4 | 1.357 | 17.549 | 16.491 | −0.62 | +0.95 | +0.396 | 8.626 |
| ATOM | 6 | C10 | UD1 | 4 | 2.640 | 18.106 | 16.807 | −0.39 | +0.00 | +0.000 | 8.626 |
| ATOM | 7 | C11 | UD1 | 4 | 3.587 | 17.315 | 17.351 | −0.41 | +0.00 | +0.000 | 8.626 |
| ATOM | 8 | O6 | UD1 | 4 | 1.920 | 14.172 | 17.575 | −0.22 | −0.61 | −0.396 | 8.626 |
| ATOM | 9 | O7 | UD1 | 4 | 0.412 | 18.165 | 15.989 | −0.27 | −0.98 | −0.396 | 8.626 |
| ATOM | 10 | C12 | UD1 | 4 | 4.437 | 15.138 | 18.223 | −0.58 | +0.49 | +0.324 | 8.626 |
| ATOM | 11 | C13 | UD1 | 4 | 5.800 | 15.224 | 17.530 | −0.65 | +0.16 | +0.113 | 8.626 |
| ATOM | 12 | C14 | UD1 | 4 | 6.741 | 14.815 | 18.661 | −0.66 | +0.17 | +0.113 | 8.626 |
| ATOM | 13 | C15 | UD1 | 4 | 6.016 | 15.264 | 19.935 | −0.46 | +0.17 | +0.113 | 8.626 |
| ATOM | 14 | O9 | UD1 | 4 | 4.653 | 15.569 | 19.550 | +0.02 | −0.34 | −0.227 | 8.626 |
| ATOM | 15 | O8 | UD1 | 4 | 5.853 | 14.398 | 16.384 | −0.49 | −0.84 | −0.537 | 8.626 |
| ATOM | 16 | H8 | UD1 | 4 | 5.995 | 14.980 | 15.546 | −0.56 | +0.35 | +0.424 | 8.626 |
| ATOM | 17 | O10 | UD1 | 4 | 7.051 | 13.418 | 18.706 | −0.17 | −0.91 | −0.537 | 8.626 |
| ATOM | 18 | HO10 | UD1 | 4 | 6.295 | 12.883 | 18.255 | +0.05 | +0.75 | +0.424 | 8.626 |
| ATOM | 19 | C16 | UD1 | 4 | 6.600 | 16.513 | 20.553 | −0.48 | +0.17 | +0.113 | 8.626 |
| ATOM | 20 | O11 | UD1 | 4 | 6.267 | 16.571 | 21.946 | −0.03 | −0.54 | −0.368 | 8.626 |
| ATOM | 21 | PA | UD1 | 4 | 5.365 | 17.752 | 22.545 | −0.54 | +1.76 | +1.019 | 8.626 |
| ATOM | 22 | O1A | UD1 | 4 | 4.694 | 17.147 | 23.717 | −0.10 | −0.42 | −0.255 | 8.626 |
| ATOM | 23 | O2A | UD1 | 4 | 4.372 | 18.171 | 21.524 | +0.02 | −0.47 | −0.255 | 8.626 |
| ATOM | 24 | O3A | UD1 | 4 | 6.297 | 18.869 | 22.917 | −0.06 | −1.08 | −0.510 | 8.626 |
| ATOM | 25 | PB | UD1 | 4 | 7.495 | 19.008 | 23.934 | −0.68 | +2.06 | +1.019 | 8.626 |
| ATOM | 26 | O1B | UD1 | 4 | 8.407 | 17.842 | 23.862 | −0.13 | −0.40 | −0.255 | 8.626 |
| ATOM | 27 | O2B | UD1 | 4 | 7.010 | 19.295 | 25.311 | −0.22 | −0.57 | −0.255 | 8.626 |
| ATOM | 28 | O | UD1 | 4 | 8.256 | 20.359 | 23.538 | −0.14 | −0.59 | −0.255 | 8.626 |
| TER | | | | | | | | | | | |
| ENDMDL | | | | | | | | | | | |
| MODEL | 5 | | | | | | | | | | |
| USER | Run = 5 | | | | | | | | | | |
| USER | Cluster Rank = 5 | | | | | | | | | | |
| USER | Number of conformations in this cluster = 1 | | | | | | | | | | |
| USER | | | | | | | | | | | |
| USER | RMSD from reference structure = 8.587 A | | | | | | | | | | |
| USER | | | | | | | | | | | |
| USER | Estimated Free Energy of Binding = −7.45 kcal/mol [= (1) + (3)] | | | | | | | | | | |
| USER | Estimated Inhibition Constant, Ki = +3.45e−06 [Temperature = 298.15 K] | | | | | | | | | | |
| USER | | | | | | | | | | | |
| USER | Final Docked Energy = −10.28 kcal/mol [= (1) + (2)] | | | | | | | | | | |
| USER | | | | | | | | | | | |
| USER | (1) Final Intermolecular Energy = −9.63 kcal/mol | | | | | | | | | | |
| USER | (2) Final Internal Energy of Ligand = −0.65 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 4.070648 14.903325 18.004813 | | | | | | | | | | |
| USER | NEWDPF quat0 −0.481332 −0.652037 0.585805 166.647551 | | | | | | | | | | |
| USER | NEWDPF ndihe 7 | | | | | | | | | | |
| USER | NEWDPF dihe0 −58.14 −180.00 111.23 −109.19 91.71 15.34 179.95 | | | | | | | | | | |
| USER | | | | | | | | | | | |
| USER | | | | Rank | x | y | z | vdW | Elec | q | RMS |
| ATOM | 1 | N1 | UD1 | 5 | 5.998 | 15.961 | 18.808 | −0.26 | −0.32 | −0.211 | 8.587 |
| ATOM | 2 | C8 | UD1 | 5 | 7.192 | 15.308 | 19.116 | −0.66 | +0.61 | +0.396 | 8.587 |
| ATOM | 3 | N2 | UD1 | 5 | 8.328 | 15.981 | 18.735 | −0.49 | −0.75 | −0.440 | 8.587 |
| ATOM | 4 | H2 | UD1 | 5 | 9.225 | 15.520 | 18.948 | +0.00 | +0.86 | +0.440 | 8.587 |
| ATOM | 5 | C9 | UD1 | 5 | 8.403 | 17.219 | 18.092 | −0.80 | +0.71 | +0.396 | 8.587 |
| ATOM | 6 | C10 | UD1 | 5 | 7.137 | 17.830 | 17.806 | −0.45 | +0.00 | +0.000 | 8.587 |
| ATOM | 7 | C11 | UD1 | 5 | 6.002 | 17.197 | 18.166 | −0.42 | +0.00 | +0.000 | 8.587 |
| ATOM | 8 | O6 | UD1 | 5 | 7.238 | 14.217 | 19.680 | −0.05 | −0.63 | −0.396 | 8.587 |
| ATOM | 9 | O7 | UD1 | 5 | 9.507 | 17.697 | 17.819 | −0.30 | −0.77 | −0.396 | 8.587 |
| ATOM | 10 | C12 | UD1 | 5 | 4.737 | 15.303 | 19.183 | −0.49 | +0.48 | +0.324 | 8.587 |
| ATOM | 11 | C13 | UD1 | 5 | 3.743 | 16.183 | 19.946 | −0.49 | +0.18 | +0.113 | 8.587 |
| ATOM | 12 | C14 | UD1 | 5 | 2.420 | 15.487 | 19.634 | −0.73 | +0.19 | +0.113 | 8.587 |
| ATOM | 13 | C15 | UD1 | 5 | 2.644 | 14.852 | 18.256 | −0.70 | +0.18 | +0.113 | 8.587 |
| ATOM | 14 | O9 | UD1 | 5 | 4.071 | 14.903 | 18.005 | −0.06 | −0.35 | −0.227 | 8.587 |
| ATOM | 15 | O8 | UD1 | 5 | 4.065 | 16.260 | 21.321 | −0.08 | −0.79 | −0.537 | 8.587 |
| ATOM | 16 | H8 | UD1 | 5 | 4.202 | 15.311 | 21.696 | +0.05 | +0.52 | +0.424 | 8.587 |
| ATOM | 17 | O10 | UD1 | 5 | 2.018 | 14.506 | 20.598 | −0.18 | −0.93 | −0.537 | 8.587 |
| ATOM | 18 | HO10 | UD1 | 5 | 1.365 | 14.932 | 21.270 | −0.17 | +0.90 | +0.424 | 8.587 |
| ATOM | 19 | C16 | UD1 | 5 | 1.961 | 15.594 | 17.132 | −0.80 | +0.20 | +0.113 | 8.587 |
| ATOM | 20 | O11 | UD1 | 5 | 2.710 | 16.769 | 16.795 | −0.35 | −0.85 | −0.368 | 8.587 |
| ATOM | 21 | PA | UD1 | 5 | 2.045 | 18.225 | 16.823 | −0.48 | +2.43 | +1.019 | 8.587 |
| ATOM | 22 | O1A | UD1 | 5 | 3.179 | 19.153 | 16.614 | +0.02 | −0.55 | −0.255 | 8.587 |
| ATOM | 23 | O2A | UD1 | 5 | 1.416 | 18.448 | 18.149 | +0.00 | −0.69 | −0.255 | 8.587 |
| ATOM | 24 | O3A | UD1 | 5 | 1.041 | 18.270 | 15.707 | −0.30 | −1.38 | −0.510 | 8.587 |
| ATOM | 25 | PB | UD1 | 5 | −0.219 | 19.167 | 15.398 | −0.60 | +2.35 | +1.019 | 8.587 |
| ATOM | 26 | O1B | UD1 | 5 | −1.422 | 18.343 | 15.135 | −0.39 | −0.62 | −0.255 | 8.587 |
| ATOM | 27 | O2B | UD1 | 5 | 0.067 | 20.155 | 14.324 | +0.00 | −0.53 | −0.255 | 8.587 |
| ATOM | 28 | O | UD1 | 5 | −0.428 | 20.095 | 16.685 | −0.27 | −0.65 | −0.255 | 8.587 |
| TER | | | | | | | | | | | |
| ENDMDL | | | | | | | | | | | |
| MODEL | 2 | | | | | | | | | | |
| USER | Run = 2 | | | | | | | | | | |
| USER | Cluster Rank = 6 | | | | | | | | | | |
| USER | Number of conformations in this cluster = 1 | | | | | | | | | | |
| USER | | | | | | | | | | | |
| USER | RMSD from reference structure = 6.324 A | | | | | | | | | | |
| USER | | | | | | | | | | | |
| USER | Estimated Free Energy of Binding = −7.48 kcal/mol [= (1) + (3)] | | | | | | | | | | |
| USER | Estimated Inhibition Constant, Ki = +3.27e−06 [Temperature = 298.15 K] | | | | | | | | | | |
| USER | | | | | | | | | | | |
| USER | Final Docked Energy = −9.91 kcal/mol [= (1) + (2)] | | | | | | | | | | |
| USER | | | | | | | | | | | |
| USER | (1) Final Intermolecular Energy = −9.66 kcal/mol | | | | | | | | | | |
| USER | (2) Final Internal Energy of Ligand = −0.25 kcal/mol | | | | | | | | | | |
| USER | (3) Torsional Free Energy = +2.18 kcal/mol | | | | | | | | | | |
| USER | | | | | | | | | | | |
| USER | | | | | | | | | | | |
| USER | DPF = test.dpf | | | | | | | | | | |
| USER | NEWDPF move m_udp.pdbq | | | | | | | | | | |
| USER | NEWDPF about −1.182000 20.257999 9.670000 | | | | | | | | | | |
| USER | NEWDPF tran0 0.122584 17.840770 16.939086 | | | | | | | | | | |
| USER | NEWDPF quat0 0.703706 −0.228448 −0.672763 −157.282277 | | | | | | | | | | |
| USER | NEWDPF ndihe 7 | | | | | | | | | | |
| USER | NEWDPF dihe0 −97.11 −85.18 −176.90 −165.81 72.22 3.63 132.07 | | | | | | | | | | |
| USER | | | | | | | | | | | |
| USER | | | | Rank | x | y | z | vdW | Elec | q | RMS |
| ATOM | 1 | N1 | UD1 | 6 | 1.926 | 16.494 | 17.582 | −0.45 | −0.41 | −0.211 | 6.324 |
| ATOM | 2 | C8 | UD1 | 6 | 2.086 | 15.935 | 18.850 | −0.72 | +0.71 | +0.396 | 6.324 |
| ATOM | 3 | N2 | UD1 | 6 | 2.563 | 14.646 | 18.852 | −0.36 | −0.71 | −0.440 | 6.324 |
| ATOM | 4 | H2 | UD1 | 6 | 2.688 | 14.206 | 19.775 | +0.05 | +0.71 | +0.440 | 6.324 |
| ATOM | 5 | C9 | UD1 | 6 | 2.895 | 13.865 | 17.741 | −0.82 | +0.60 | +0.396 | 6.324 |
| ATOM | 6 | C10 | UD1 | 6 | 2.704 | 14.505 | 16.472 | −0.92 | +0.00 | +0.000 | 6.324 |
| ATOM | 7 | C11 | UD1 | 6 | 2.238 | 15.769 | 16.434 | −0.50 | +0.00 | +0.000 | 6.324 |
| ATOM | 8 | O6 | UD1 | 6 | 1.824 | 16.534 | 19.891 | −0.17 | −0.81 | −0.396 | 6.324 |
| ATOM | 9 | O7 | UD1 | 6 | 3.318 | 12.718 | 17.908 | −0.22 | −0.60 | −0.396 | 6.324 |
| ATOM | 10 | C12 | UD1 | 6 | 1.418 | 17.873 | 17.497 | −0.58 | +0.77 | +0.324 | 6.324 |
| ATOM | 11 | C13 | UD1 | 6 | 2.233 | 18.814 | 16.606 | −0.44 | +0.27 | +0.113 | 6.324 |
| ATOM | 12 | C14 | UD1 | 6 | 1.187 | 19.865 | 16.239 | −0.45 | +0.26 | +0.113 | 6.324 |
| ATOM | 13 | C15 | UD1 | 6 | −0.142 | 19.101 | 16.273 | −0.52 | +0.27 | +0.113 | 6.324 |
| ATOM | 14 | O9 | UD1 | 6 | 0.123 | 17.841 | 16.939 | −0.09 | −0.56 | −0.227 | 6.324 |
| ATOM | 15 | O8 | UD1 | 6 | 3.369 | 19.317 | 17.282 | +0.08 | −1.10 | −0.537 | 6.324 |
| ATOM | 16 | H8 | UD1 | 6 | 3.120 | 20.188 | 17.772 | +0.10 | +0.87 | +0.424 | 6.324 |
| ATOM | 17 | O10 | UD1 | 6 | 1.152 | 21.004 | 17.107 | −0.13 | −1.24 | −0.537 | 6.324 |
| ATOM | 18 | HO10 | UD1 | 6 | 0.315 | 21.567 | 16.902 | +0.08 | +0.92 | +0.424 | 6.324 |
| ATOM | 19 | C16 | UD1 | 6 | −0.689 | 18.780 | 14.901 | −0.60 | +0.27 | +0.113 | 6.324 |
| ATOM | 20 | O11 | UD1 | 6 | 0.390 | 18.629 | 13.969 | −0.30 | −1.22 | −0.368 | 6.324 |
| ATOM | 21 | PA | UD1 | 6 | 0.340 | 19.295 | 12.513 | −0.48 | +2.50 | +1.019 | 6.324 |
| ATOM | 22 | O1A | UD1 | 6 | 1.759 | 19.514 | 12.158 | −0.42 | −0.73 | −0.255 | 6.324 |
| ATOM | 23 | O2A | UD1 | 6 | −0.390 | 20.586 | 12.594 | −0.03 | −0.46 | −0.255 | 6.324 |
| ATOM | 24 | O3A | UD1 | 6 | −0.346 | 18.315 | 11.606 | −0.11 | −1.02 | −0.510 | 6.324 |
| ATOM | 25 | PB | UD1 | 6 | −1.795 | 18.213 | 10.992 | −0.73 | +1.73 | +1.019 | 6.324 |
| ATOM | 26 | O1B | UD1 | 6 | −2.835 | 18.247 | 12.046 | −0.16 | −0.45 | −0.255 | 6.324 |
| ATOM | 27 | O2B | UD1 | 6 | −1.919 | 17.046 | 10.078 | −0.25 | −0.33 | −0.255 | 6.324 |
| ATOM | 28 | O | UD1 | 6 | −1.942 | 19.458 | 9.998 | −0.22 | −0.54 | −0.255 | 6.324 |
| TER | | | | | | | | | | | |
| ENDMDL | | | | | | | | | | | |

TABLE 17
CLUSTERING HISTOGRAM

| | Lowest | | Mean | Num | | | | | | | |
|---|---|---|---|---|---|---|---|---|---|---|---|
| Cluster | Docked | | Docked | in | Histogram | | | | | | |
| Rank | Energy | Run | Energy | Clus | 5 | 10 | 15 | 20 | 25 | 30 | 35 |
| 1 | −15.93 | 74 | −15.07 | 2 | ## | | | | | | |
| 2 | −14.82 | 86 | −14.82 | 1 | # | | | | | | |
| 3 | −14.49 | 71 | −14.49 | 1 | # | | | | | | |
| 4 | −14.27 | 15 | −14.27 | 1 | # | | | | | | |
| 5 | −14.04 | 82 | −14.04 | 1 | # | | | | | | |
| 6 | −14.02 | 67 | −14.02 | 1 | # | | | | | | |
| 7 | −13.98 | 55 | −13.98 | 1 | # | | | | | | |
| 8 | −13.86 | 72 | −11.86 | 2 | ## | | | | | | |
| 9 | −13.83 | 90 | −13.83 | 1 | # | | | | | | |
| 10 | −13.67 | 66 | −13.67 | 1 | # | | | | | | |

LOWEST ENERGY DOCKED CONFORMATION from EACH CLUSTER
Residue number will be set to the conformation's cluster rank.

| MODEL | 74 | | | | | | | | | | |
|---|---|---|---|---|---|---|---|---|---|---|---|
| USER | Run = 74 | | | | | | | | | | |
| USER | Cluster Rank = 1 | | | | | | | | | | |
| USER | Number of conformations in this cluster = 2 | | | | | | | | | | |
| USER | | | | | | | | | | | |
| USER | RMSD from reference structure = 1.625 A | | | | | | | | | | |
| USER | | | | | | | | | | | |
| USER | Estimated Free Energy of Binding = −12.17 kcal/mol [= (1) + (3)] | | | | | | | | | | |
| USER | Estimated Inhibition Constant, Ki = +1.19e−09 [Temperature = 298.15 K] | | | | | | | | | | |
| USER | | | | | | | | | | | |
| USER | Final Docked Energy = −15.93 kcal/mol [= (1) + (2)] | | | | | | | | | | |
| USER | | | | | | | | | | | |
| USER | (1) Final Intermolecular Energy = −16.53 kcal/mol | | | | | | | | | | |
| USER | (2) Final Internal Energy of Ligand = +0.60 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 0.599466 19.158218 13.267607 | | | | | | | | | | |
| USER | NEWDPF quat0 0.972175 0.233954 −0.011893 −10.711334 | | | | | | | | | | |
| USER | NEWDPF ndihe 14 | | | | | | | | | | |
| USER | NEWDPF dihe0 179.90 179.99 −96.55 −93.70 −177.20 −158.17 174.40 −38.27 | | | | | | | | | | |
| | 28.40 40.85 −111.90 −1.16 64.91 2.21 | | | | | | | | | | |
| USER | | | | | | | | | | | |
| USER | | | | Rank | x | y | z | vdW | Elec | q | RMS |
| ATOM | 1 | N1 | MOL | 1 | −0.542 | 19.605 | 7.026 | −0.35 | −0.19 | −0.211 | 1.625 |
| ATOM | 2 | C8 | MOL | 1 | 0.263 | 20.673 | 6.650 | −0.54 | +0.00 | +0.000 | 1.625 |
| ATOM | 3 | C6 | MOL | 1 | −0.188 | 21.713 | 5.933 | −0.62 | +0.00 | +0.000 | 1.625 |
| ATOM | 4 | C5 | MOL | 1 | −1.549 | 21.731 | 5.497 | −0.72 | +0.21 | +0.396 | 1.625 |
| ATOM | 5 | O11 | MOL | 1 | −2.110 | 22.609 | 4.841 | −0.07 | −0.14 | −0.396 | 1.625 |
| ATOM | 6 | N3 | MOL | 1 | −2.301 | 20.626 | 5.897 | −0.53 | −0.28 | −0.440 | 1.625 |
| ATOM | 7 | H3 | MOL | 1 | −3.291 | 20.610 | 5.612 | −0.28 | +0.11 | +0.440 | 1.625 |
| ATOM | 8 | C2 | MOL | 1 | −1.851 | 19.561 | 6.634 | −0.79 | +0.42 | +0.396 | 1.625 |
| ATOM | 9 | O10 | MOL | 1 | −2.589 | 18.623 | 6.940 | −0.52 | −0.67 | −0.396 | 1.625 |
| ATOM | 10 | C12 | MOL | 1 | 0.001 | 18.503 | 7.833 | −0.55 | +0.30 | +0.324 | 1.625 |
| ATOM | 11 | O22 | MOL | 1 | 0.375 | 19.070 | 9.084 | +0.00 | −0.23 | −0.227 | 1.625 |
| ATOM | 12 | C20 | MOL | 1 | 1.368 | 18.219 | 9.718 | −0.52 | +0.12 | +0.113 | 1.625 |
| ATOM | 13 | C25 | MOL | 1 | 2.465 | 19.105 | 10.309 | −0.49 | +0.14 | +0.113 | 1.625 |
| ATOM | 14 | O28 | MOL | 1 | 2.573 | 18.802 | 11.687 | +0.03 | −0.45 | −0.368 | 1.625 |
| ATOM | 15 | P29 | MOL | 1 | 2.784 | 19.684 | 12.977 | −0.41 | +1.10 | +1.019 | 1.625 |
| ATOM | 16 | O30 | MOL | 1 | 4.033 | 19.220 | 13.645 | +0.09 | −0.27 | −0.255 | 1.625 |
| ATOM | 17 | O31 | MOL | 1 | 2.680 | 21.127 | 12.591 | −0.08 | −0.28 | −0.255 | 1.625 |
| ATOM | 18 | O32 | MOL | 1 | 1.513 | 19.121 | 13.760 | −0.03 | −0.58 | −0.510 | 1.625 |
| ATOM | 19 | P33 | MOL | 1 | 0.113 | 19.769 | 14.180 | −0.60 | +1.20 | +1.019 | 1.625 |
| ATOM | 20 | O35 | MOL | 1 | −0.660 | 19.607 | 12.922 | −0.27 | −0.39 | −0.255 | 1.625 |
| ATOM | 21 | O36 | MOL | 1 | 0.219 | 21.100 | 14.828 | −0.12 | −0.21 | −0.255 | 1.625 |
| ATOM | 22 | O34 | MOL | 1 | −0.451 | 18.803 | 15.351 | −0.20 | −0.37 | −0.368 | 1.625 |
| ATOM | 23 | C37 | MOL | 1 | 1.157 | 16.647 | 15.594 | −0.39 | +0.21 | +0.227 | 1.625 |
| ATOM | 24 | O39 | MOL | 1 | 2.042 | 17.425 | 16.021 | +0.04 | −0.18 | −0.227 | 1.625 |
| ATOM | 25 | C40 | MOL | 1 | 1.987 | 18.072 | 17.313 | −0.25 | +0.05 | +0.113 | 1.625 |
| ATOM | 26 | C50 | MOL | 1 | 3.394 | 18.600 | 17.522 | −0.29 | +0.08 | +0.113 | 1.625 |
| ATOM | 27 | O61 | MOL | 1 | 3.451 | 19.458 | 18.606 | +0.06 | −0.38 | −0.537 | 1.625 |
| ATOM | 28 | H61 | MOL | 1 | 2.540 | 19.620 | 18.897 | −0.21 | +0.11 | +0.424 | 1.625 |
| ATOM | 29 | C41 | MOL | 1 | 1.541 | 17.040 | 18.334 | −0.57 | −0.08 | +0.113 | 1.625 |
| ATOM | 30 | O48 | MOL | 1 | 1.335 | 17.630 | 19.594 | −0.27 | −0.56 | −0.537 | 1.625 |
| ATOM | 31 | H48 | MOL | 1 | 0.659 | 18.288 | 19.529 | −0.26 | +0.10 | +0.424 | 1.625 |
| ATOM | 32 | C42 | MOL | 1 | 0.213 | 16.442 | 17.902 | −0.73 | +0.10 | +0.113 | 1.625 |
| ATOM | 33 | O46 | MOL | 1 | −0.222 | 15.470 | 18.804 | −0.22 | −0.62 | −0.537 | 1.625 |
| ATOM | 34 | H46 | MOL | 1 | 0.214 | 15.635 | 19.631 | −0.16 | +0.31 | −0.424 | 1.625 |
| ATOM | 35 | C43 | MOL | 1 | 0.369 | 15.772 | 16.537 | −0.65 | +0.23 | +0.211 | 1.625 |
| ATOM | 36 | N51 | MOL | 1 | −0.888 | 15.369 | 15.977 | −0.56 | −0.88 | −0.650 | 1.625 |
| ATOM | 37 | H51 | MOL | 1 | −1.601 | 16.091 | 15.801 | +0.07 | +0.64 | −0.440 | 1.625 |
| ATOM | 38 | C53 | MOL | 1 | −1.179 | 14.081 | 15.667 | −0.89 | +0.63 | +0.396 | 1.625 |
| ATOM | 39 | O54 | MOL | 1 | −0.372 | 13.195 | 15.693 | −0.02 | −0.65 | −0.396 | 1.625 |
| ATOM | 40 | C55 | MOL | 1 | −2.629 | 13.838 | 15.309 | −0.67 | +0.00 | +0.000 | 1.625 |
| ATOM | 41 | C18 | MOL | 1 | 1.878 | 17.294 | 8.612 | −0.49 | +0.11 | +0.113 | 1.625 |
| ATOM | 42 | O23 | MOL | 1 | 1.266 | 16.007 | 8.741 | −0.12 | −0.32 | −0.537 | 1.625 |
| ATOM | 43 | H23 | MOL | 1 | 1.053 | 15.633 | 7.804 | −0.21 | +0.00 | −0.424 | 1.625 |
| ATOM | 44 | C14 | MOL | 1 | 1.323 | 17.930 | 7.316 | −0.56 | +0.10 | +0.113 | 1.625 |
| ATOM | 45 | O16 | MOL | 1 | 1.085 | 16.929 | 6.355 | −0.17 | −0.36 | −0.537 | 1.625 |
| ATOM | 46 | H16 | MOL | 1 | 0.441 | 16.226 | 6.744 | +0.01 | +0.20 | +0.424 | 1.625 |
| TER | | | | | | | | | | | |
| ENDMDL | | | | | | | | | | | |
| MODEL | 13 | | | | | | | | | | |
| USER | Run = 13 | | | | | | | | | | |
| USER | Cluster Rank = 1 | | | | | | | | | | |
| USER | Number of conformations in this cluster = 2 | | | | | | | | | | |
| USER | | | | | | | | | | | |
| USER | RMSD from reference structure = 1.728 A | | | | | | | | | | |
| USER | | | | | | | | | | | |
| USER | Estimated Free Energy of Binding = −10.31 kcal/mol [= (1) + (3)] | | | | | | | | | | |
| USER | Estimated Inhibition Constant, Ki = +2.75e−08 [Temperature = 298.15 K] | | | | | | | | | | |
| USER | | | | | | | | | | | |
| USER | Final Docked Energy = −14.21 kcal/mol [= (1) + (2)] | | | | | | | | | | |
| USER | | | | | | | | | | | |
| USER | (1) Final Intermolecular Energy = −14.67 kcal/mol | | | | | | | | | | |
| USER | (2) Final Internal Energy of Ligand = +0.47 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 0.239632 19.390169 12.990590 | | | | | | | | | | |
| USER | NEWDPF quat0 −0.646734 −0.649829 0.399322 14.597981 | | | | | | | | | | |
| USER | NEWDPF ndihe 14 | | | | | | | | | | |
| USER | NEWDPF dihe0 172.13 −102.40 178.85 −111.85 −22.15 107.13 153.11 −36.41 | | | | | | | | | | |
| | 19.36 39.05 −57.47 81.23 −26.13 −20.57 | | | | | | | | | | |
| USER | | | | | | | | | | | |
| USER | | | | Rank | x | y | z | vdW | Elec | q | RMS |
| ATOM | 1 | N1 | MOL | 1 | −0.245 | 19.837 | 6.664 | −0.34 | −0.18 | −0.211 | 1.728 |
| ATOM | 2 | C8 | MOL | 1 | 0.474 | 20.994 | 6.390 | −0.53 | +0.00 | +0.000 | 1.728 |
| ATOM | 3 | C6 | MOL | 1 | −0.007 | 21.994 | 5.635 | −0.60 | +0.00 | +0.000 | 1.728 |
| ATOM | 4 | C5 | MOL | 1 | −1.305 | 21.872 | 5.048 | −0.64 | +0.20 | +0.396 | 1.728 |
| ATOM | 5 | O11 | MOL | 1 | −1.883 | 22.697 | 4.341 | −0.13 | −0.16 | −0.396 | 1.728 |
| ATOM | 6 | N3 | MOL | 1 | −1.970 | 20.684 | 5.353 | −0.44 | −0.22 | −0.440 | 1.728 |
| ATOM | 7 | H3 | MOL | 1 | −2.914 | 20.566 | 4.958 | −0.27 | −0.07 | +0.440 | 1.728 |
| ATOM | 8 | C2 | MOL | 1 | −1.490 | 19.659 | 6.127 | −0.74 | +0.36 | +0.396 | 1.728 |
| ATOM | 9 | O10 | MOL | 1 | −2.149 | 18.640 | 6.340 | −0.47 | −0.55 | −0.396 | 1.728 |
| ATOM | 10 | C12 | MOL | 1 | 0.324 | 18.785 | 7.518 | −0.53 | +0.29 | +0.324 | 1.728 |
| ATOM | 11 | O22 | MOL | 1 | 0.492 | 19.364 | 8.808 | −0.04 | −0.22 | −0.227 | 1.728 |
| ATOM | 12 | C20 | MOL | 1 | 1.496 | 18.613 | 9.542 | −0.50 | −0.12 | +0.113 | 1.728 |
| ATOM | 13 | C25 | MOL | 1 | 2.416 | 19.602 | 10.260 | −0.24 | +0.14 | +0.113 | 1.728 |
| ATOM | 14 | O28 | MOL | 1 | 2.021 | 19.654 | 11.618 | +0.10 | −0.43 | −0.368 | 1.728 |
| ATOM | 15 | P29 | MOL | 1 | 2.807 | 19.521 | 12.979 | −0.39 | +1.11 | +1.019 | 1.728 |
| ATOM | 16 | O30 | MOL | 1 | 3.841 | 18.461 | 12.806 | +0.06 | −0.33 | −0.255 | 1.728 |
| ATOM | 17 | O31 | MOL | 1 | 3.252 | 20.884 | 13.409 | −0.04 | −0.25 | −0.255 | 1.728 |
| ATOM | 18 | O32 | MOL | 1 | 1.575 | 18.941 | 13.813 | −0.05 | −0.58 | −0.510 | 1.728 |
| ATOM | 19 | P33 | MOL | 1 | 0.096 | 19.479 | 14.095 | −0.58 | +1.30 | +1.019 | 1.728 |
| ATOM | 20 | O35 | MOL | 1 | −0.592 | 19.078 | 12.842 | −0.35 | −0.40 | −0.255 | 1.728 |
| ATOM | 21 | O36 | MOL | 1 | 0.032 | 20.884 | 14.570 | −0.13 | −0.24 | −0.255 | 1.728 |
| ATOM | 22 | O34 | MOL | 1 | −0.421 | 18.609 | 15.360 | −0.20 | −0.38 | −0.368 | 1.728 |
| ATOM | 23 | C37 | MOL | 1 | 1.175 | 16.457 | 15.689 | −0.41 | +0.21 | +0.227 | 1.728 |
| ATOM | 24 | O39 | MOL | 1 | 2.075 | 17.249 | 16.054 | +0.04 | −0.18 | −0.227 | 1.728 |
| ATOM | 25 | C40 | MOL | 1 | 2.052 | 17.967 | 17.308 | −0.31 | +0.06 | +0.113 | 1.728 |
| ATOM | 26 | C50 | MOL | 1 | 3.467 | 18.493 | 17.459 | −0.30 | +0.08 | +0.113 | 1.728 |
| ATOM | 27 | O61 | MOL | 1 | 3.699 | 19.559 | 16.608 | +0.13 | −0.39 | −0.537 | 1.728 |
| ATOM | 28 | H61 | MOL | 1 | 2.996 | 19.556 | 15.940 | +0.11 | +0.30 | +0.424 | 1.728 |
| ATOM | 29 | C41 | MOL | 1 | 1.620 | 16.996 | 18.394 | −0.57 | +0.08 | +0.113 | 1.728 |
| ATOM | 30 | O48 | MOL | 1 | 1.445 | 17.657 | 19.624 | −0.29 | −0.59 | −0.537 | 1.728 |
| ATOM | 31 | H48 | MOL | 1 | 0.667 | 17.329 | 20.051 | +0.02 | +0.55 | −0.424 | 1.728 |
| ATOM | 32 | C42 | MOL | 1 | 0.278 | 16.387 | 18.023 | −0.74 | +0.10 | +0.113 | 1.728 |
| ATOM | 33 | O46 | MOL | 1 | −0.145 | 15.470 | 18.986 | −0.13 | −0.57 | −0.537 | 1.728 |
| ATOM | 34 | H46 | MOL | 1 | 0.489 | 14.764 | 19.005 | −0.24 | +0.40 | +0.424 | 1.728 |
| ATOM | 35 | C43 | MOL | 1 | 0.400 | 15.641 | 16.694 | −0.63 | +0.24 | +0.211 | 1.728 |
| ATOM | 36 | N51 | MOL | 1 | −0.871 | 15.220 | 16.183 | −0.58 | −0.93 | −0.650 | 1.728 |
| ATOM | 37 | H51 | MOL | 1 | −1.678 | 15.850 | 16.291 | +0.04 | +0.63 | +0.440 | 1.728 |
| ATOM | 38 | C53 | MOL | 1 | −1.059 | 14.027 | 15.563 | −0.86 | +0.62 | +0.396 | 1.728 |
| ATOM | 39 | O54 | MOL | 1 | −0.163 | 13.293 | 15.257 | −0.28 | −0.57 | −0.396 | 1.728 |
| ATOM | 40 | C55 | MOL | 1 | −2.509 | 13.677 | 15.310 | −0.81 | +0.00 | +0.000 | 1.728 |
| ATOM | 41 | C18 | MOL | 1 | 2.225 | 17.772 | 8.493 | −0.48 | +0.12 | +0.113 | 1.728 |
| ATOM | 42 | O23 | MOL | 1 | 1.749 | 16.424 | 8.542 | −0.07 | −0.42 | −0.537 | 1.728 |
| ATOM | 43 | H23 | MOL | 1 | 1.396 | 16.151 | 7.613 | −0.15 | +0.16 | +0.424 | 1.728 |
| ATOM | 44 | C14 | MOL | 1 | 1.751 | 18.371 | 7.149 | −0.54 | +0.10 | +0.113 | 1.728 |
| ATOM | 45 | O16 | MOL | 1 | 1.732 | 17.370 | 6.159 | −0.14 | −0.41 | −0.537 | 1.728 |
| ATOM | 46 | H16 | MOL | 1 | 1.946 | 17.789 | 5.242 | +0.06 | +0.36 | +0.424 | 1.728 |
| TER | | | | | | | | | | | |
| ENDMOL | | | | | | | | | | | |
| MODEL | 86 | | | | | | | | | | |
| USER | Run = 86 | | | | | | | | | | |
| USER | Cluster Rank = 2 | | | | | | | | | | |
| USER | Number of conformations in this cluster = 1 | | | | | | | | | | |
| USER | | | | | | | | | | | |
| USER | RMSD from reference structure = 2.106 A | | | | | | | | | | |
| USER | | | | | | | | | | | |
| USER | Estimated Free Energy of Binding = −11.06 kcal/mol [= (1) + (3)] | | | | | | | | | | |
| USER | Estimated Inhibition Constant, Ki = +7.86e−09 [Temperature 298.15 K] | | | | | | | | | | |
| USER | | | | | | | | | | | |
| USER | Final Docked Energy = −14.82 kcal/mol [= (1) + (2)] | | | | | | | | | | |
| USER | | | | | | | | | | | |
| USER | (1) Final Intermolecular Energy = −15.41 kcal/mol | | | | | | | | | | |
| USER | (2) Final Internal Energy of Ligand = +0.59 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −2.629603 18.598510 12.778890 | | | | | | | | | | |
| USER | NEWDPF quat0 0.240744 0.891367 0.384067 −30.553694 | | | | | | | | | | |
| USER | NEWDPF ndihe 14 | | | | | | | | | | |
| USER | NEWDPF dihe0 166.12 180.00 −179.69 −29.10 78.48 178.55 −166.94 40.75 | | | | | | | | | | |
| | −27.36 97.83 151.59 166.60 93.29 172.74 | | | | | | | | | | |
| USER | | | | | | | | | | | |
| USER | | | | Rank | x | y | z | vdW | Elec | q | RMS |
| ATOM | 1 | N1 | MOL | 2 | −0.859 | 19.332 | 6.714 | −0.38 | −0.20 | −0.211 | 2.106 |
| ATOM | 2 | C8 | MOL | 2 | 0.178 | 20.252 | 6.814 | −0.55 | +0.00 | +0.000 | 2.106 |
| ATOM | 3 | C6 | MOL | 2 | 0.243 | 21.366 | 6.068 | −0.57 | +0.00 | +0.000 | 2.106 |
| ATOM | 4 | C5 | MOL | 2 | −0.773 | 21.623 | 5.096 | −0.56 | +0.23 | +0.396 | 2.106 |
| ATOM | 5 | O11 | MOL | 2 | −0.856 | 22.594 | 4.344 | −0.08 | −0.24 | −0.396 | 2.106 |
| ATOM | 6 | N3 | MOL | 2 | −1.778 | 20.657 | 5.037 | −0.41 | −0.20 | −0.440 | 2.106 |
| ATOM | 7 | H3 | MOL | 2 | −2.535 | 20.815 | 4.357 | −0.25 | −0.15 | +0.440 | 2.106 |
| ATOM | 8 | C2 | MOL | 2 | −1.857 | 19.519 | 5.799 | −0.77 | +0.38 | +0.396 | 2.106 |
| ATOM | 9 | O10 | MOL | 2 | −2.783 | 18.717 | 5.675 | −0.35 | −0.72 | −0.396 | 2.106 |
| ATOM | 10 | C12 | MOL | 2 | −0.890 | 18.141 | 7.575 | −0.64 | +0.32 | +0.324 | 2.106 |
| ATOM | 11 | O22 | MOL | 2 | −1.019 | 18.616 | 8.910 | −0.09 | −0.23 | −0.227 | 2.106 |
| ATOM | 12 | C20 | MOL | 2 | −0.539 | 17.598 | 9.829 | −0.66 | +0.11 | +0.113 | 2.106 |
| ATOM | 13 | C25 | MOL | 2 | 0.308 | 18.274 | 10.908 | −0.50 | +0.12 | +0.113 | 2.106 |
| ATOM | 14 | O28 | MOL | 2 | 1.458 | 18.812 | 10.283 | +0.00 | −0.40 | −0.368 | 2.106 |
| ATOM | 15 | P29 | MOL | 2 | 2.991 | 18.445 | 10.337 | −0.53 | +1.41 | +1.019 | 2.106 |
| ATOM | 16 | O30 | MOL | 2 | 3.110 | 16.968 | 10.185 | −0.37 | −0.41 | −0.255 | 2.106 |
| ATOM | 17 | O31 | MOL | 2 | 3.726 | 19.321 | 9.370 | −0.36 | −0.47 | −0.255 | 2.106 |
| ATOM | 18 | O32 | MOL | 2 | 3.192 | 18.847 | 11.868 | +0.04 | −0.66 | −0.510 | 2.106 |
| ATOM | 19 | P33 | MOL | 2 | 4.205 | 18.356 | 13.004 | −0.32 | +1.33 | +1.019 | 2.106 |
| ATOM | 20 | O35 | MOL | 2 | 4.528 | 19.645 | 13.668 | +0.06 | −0.25 | −0.255 | 2.106 |
| ATOM | 21 | O36 | MOL | 2 | 5.293 | 17.470 | 12.518 | −0.23 | −0.61 | −0.255 | 2.106 |
| ATOM | 22 | O34 | MOL | 2 | 3.315 | 17.401 | 13.962 | +0.07 | −0.37 | −0.368 | 2.106 |
| ATOM | 23 | C37 | MOL | 2 | 1.325 | 18.844 | 15.077 | −0.41 | +0.20 | +0.227 | 2.106 |
| ATOM | 24 | O39 | MOL | 2 | 0.622 | 17.807 | 15.030 | −0.22 | −0.29 | −0.227 | 2.106 |
| ATOM | 25 | C40 | MOL | 2 | 0.841 | 16.651 | 15.869 | −0.52 | +0.11 | +0.113 | 2.106 |
| ATOM | 26 | C50 | MOL | 2 | −0.429 | 15.835 | 15.712 | −0.61 | +0.14 | +0.113 | 2.106 |
| ATOM | 27 | O61 | MOL | 2 | −0.162 | 14.478 | 15.766 | −0.37 | −0.75 | −0.537 | 2.106 |
| ATOM | 28 | H61 | MOL | 2 | 0.773 | 14.356 | 15.540 | +0.05 | +0.46 | +0.424 | 2.106 |
| ATOM | 29 | C41 | MOL | 2 | 1.106 | 17.142 | 17.282 | −0.52 | +0.08 | +0.113 | 2.106 |
| ATOM | 30 | O48 | MOL | 2 | 1.460 | 16.077 | 18.130 | −0.10 | −0.46 | −0.537 | 2.106 |
| ATOM | 31 | H48 | MOL | 2 | 2.250 | 15.667 | 17.810 | +0.07 | +0.36 | +0.424 | 2.106 |
| ATOM | 32 | C42 | MOL | 2 | 2.277 | 18.109 | 17.271 | −0.30 | +0.06 | +0.113 | 2.106 |
| ATOM | 33 | O46 | MOL | 2 | 2.540 | 18.601 | 18.549 | +0.09 | −0.27 | −0.537 | 2.106 |
| ATOM | 34 | H46 | MOL | 2 | 1.766 | 19.070 | 18.837 | −0.20 | −0.21 | +0.424 | 2.106 |
| ATOM | 35 | C43 | MOL | 2 | 1.954 | 19.301 | 16.370 | −0.20 | +0.09 | +0.211 | 2.106 |
| ATOM | 36 | N51 | MOL | 2 | 3.089 | 20.146 | 16.150 | −0.16 | −0.43 | −0.650 | 2.106 |
| ATOM | 37 | H51 | MOL | 2 | 3.798 | 19.849 | 15.464 | +0.10 | +0.35 | +0.440 | 2.106 |
| ATOM | 38 | C53 | MOL | 2 | 3.274 | 21.321 | 16.803 | −0.34 | +0.26 | +0.396 | 2.106 |
| ATOM | 39 | O54 | MOL | 2 | 2.443 | 21.826 | 17.504 | +0.03 | −0.15 | −0.396 | 2.106 |
| ATOM | 40 | C55 | MOL | 2 | 4.639 | 21.941 | 16.599 | −0.29 | +0.00 | +0.000 | 2.106 |
| ATOM | 41 | C18 | MOL | 2 | 0.259 | 16.617 | 8.970 | −0.69 | +0.09 | +0.113 | 2.106 |
| ATOM | 42 | O23 | MOL | 2 | −0.529 | 15.452 | 8.711 | −0.30 | −0.37 | −0.537 | 2.106 |
| ATOM | 43 | H23 | MOL | 2 | 0.093 | 14.648 | 8.538 | −0.28 | +0.21 | +0.424 | 2.106 |
| ATOM | 44 | C14 | MOL | 2 | 0.425 | 17.359 | 7.623 | −0.62 | +0.09 | +0.113 | 2.106 |
| ATOM | 45 | O16 | MOL | 2 | 0.485 | 16.429 | 6.568 | −0.23 | −0.29 | −0.537 | 2.106 |
| ATOM | 46 | H16 | MOL | 2 | 1.148 | 15.678 | 6.808 | −0.15 | −0.06 | +0.424 | 2.106 |
| TER | | | | | | | | | | | |
| ENDMDL | | | | | | | | | | | |
| MODEL | 71 | | | | | | | | | | |
| USER | Run = 71 | | | | | | | | | | |
| USER | Cluster Rank = 3 | | | | | | | | | | |
| USER | Number of conformations in this cluster = 1 | | | | | | | | | | |
| USER | | | | | | | | | | | |
| USER | RMSD from reference structure = 2.639 A | | | | | | | | | | |
| USER | | | | | | | | | | | |
| USER | Estimated Free Energy of Binding = −10.36 kcal/mol [= (1) + (3)] | | | | | | | | | | |
| USER | Estimated Inhibition Constant, Ki = +2.53e−08 [Temperature = 298.15 K] | | | | | | | | | | |
| USER | | | | | | | | | | | |
| USER | Final Docked Energy = −14.49 kcal/mol [= (1) + (2)] | | | | | | | | | | |
| USER | | | | | | | | | | | |
| USER | (1) Final Intermolecular Energy = −14.72 kcal/mol | | | | | | | | | | |
| USER | (2) Final Internal Energy of Ligand = +0.23 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −1.433666 17.572181 13.035157 | | | | | | | | | | |
| USER | NEWDPF quat0 0.027984 −0.613581 −0.789136 23.555567 | | | | | | | | | | |
| USER | NEWDPF ndihe 14 | | | | | | | | | | |
| USER | NEWDPF dihe0 −177.88 −63.40 162.06 −96.64 58.90 4.20 −152.40 89.03 | | | | | | | | | | |
| | −120.12 −98.99 43.02 132.86 64.29 −171.52 | | | | | | | | | | |
| USER | | | | | | | | | | | |
| USER | | | | Rank | x | y | z | vdW | Elec | q | RMS |
| ATOM | 1 | N1 | MOL | 3 | −0.750 | 19.327 | 6.960 | −0.36 | −0.20 | −0.211 | 2.639 |
| ATOM | 2 | C8 | MOL | 3 | 0.382 | 20.131 | 7.006 | −0.52 | +0.00 | +0.000 | 2.639 |
| ATOM | 3 | C6 | MOL | 3 | 0.431 | 21.359 | 6.469 | −0.51 | +0.00 | +0.000 | 2.639 |
| ATOM | 4 | C5 | MOL | 3 | −0.713 | 21.874 | 5.783 | −0.68 | +0.27 | +0.396 | 2.639 |
| ATOM | 5 | O11 | MOL | 3 | −0.825 | 22.975 | 5.246 | −0.12 | −0.29 | −0.396 | 2.639 |
| ATOM | 6 | N3 | MOL | 3 | −1.812 | 21.016 | 5.763 | −0.50 | −0.26 | −0.440 | 2.639 |
| ATOM | 7 | H3 | MOL | 3 | −2.660 | 21.358 | 5.289 | −0.26 | −0.01 | +0.440 | 2.639 |
| ATOM | 8 | C2 | MOL | 3 | −1.874 | 19.761 | 6.314 | −0.79 | +0.39 | +0.396 | 2.639 |
| ATOM | 9 | O10 | MOL | 3 | −2.891 | 19.069 | 6.244 | −0.46 | −0.74 | −0.396 | 2.639 |
| ATOM | 10 | C12 | MOL | 3 | −0.753 | 17.998 | 7.588 | −0.63 | +0.31 | +0.324 | 2.639 |
| ATOM | 11 | O22 | MOL | 3 | −0.579 | 18.215 | 8.984 | −0.07 | −0.22 | −0.227 | 2.639 |
| ATOM | 12 | C20 | MOL | 3 | −0.053 | 17.006 | 9.594 | −0.65 | +0.10 | +0.113 | 2.639 |
| ATOM | 13 | C25 | MOL | 3 | 1.050 | 17.396 | 10.579 | −0.40 | +0.11 | +0.113 | 2.639 |
| ATOM | 14 | O28 | MOL | 3 | 1.940 | 18.269 | 9.910 | −0.01 | −0.42 | −0.368 | 2.639 |
| ATOM | 15 | P29 | MOL | 3 | 3.510 | 18.422 | 9.908 | −0.56 | +1.62 | +1.019 | 2.639 |
| ATOM | 16 | O30 | MOL | 3 | 4.085 | 17.213 | 10.565 | −0.65 | −0.58 | −0.255 | 2.639 |
| ATOM | 17 | O31 | MOL | 3 | 3.955 | 18.755 | 8.519 | −0.32 | −0.44 | −0.255 | 2.639 |
| ATOM | 18 | O32 | MOL | 3 | 3.547 | 19.654 | 10.921 | −0.12 | −0.76 | −0.510 | 2.639 |
| ATOM | 19 | P33 | MOL | 3 | 2.788 | 19.964 | 12.295 | −0.45 | +1.18 | +1.019 | 2.639 |
| ATOM | 20 | O35 | MOL | 3 | 1.371 | 19.866 | 11.860 | +0.66 | −0.30 | −0.255 | 2.639 |
| ATOM | 21 | O36 | MOL | 3 | 3.272 | 21.174 | 13.006 | −0.11 | −0.27 | −0.255 | 2.639 |
| ATOM | 22 | O34 | MOL | 3 | 3.195 | 18.740 | 13.273 | +0.09 | −0.41 | −0.368 | 2.639 |
| ATOM | 23 | C37 | MOL | 3 | 1.291 | 18.420 | 15.160 | −0.41 | +0.21 | +0.227 | 2.639 |
| ATOM | 24 | O39 | MOL | 3 | 2.114 | 17.717 | 15.792 | +0.06 | −0.18 | −0.227 | 2.639 |
| ATOM | 25 | C40 | MOL | 3 | 3.288 | 18.249 | 16.447 | −0.27 | +0.09 | +0.113 | 2.639 |
| ATOM | 26 | C50 | MOL | 3 | 3.753 | 17.119 | 17.347 | −0.39 | +0.10 | +0.113 | 2.639 |
| ATOM | 27 | O61 | MOL | 3 | 2.692 | 16.322 | 17.740 | +0.02 | −0.44 | −0.537 | 2.639 |
| ATOM | 28 | H61 | MOL | 3 | 1.879 | 16.740 | 17.416 | +0.08 | +0.32 | +0.424 | 2.639 |
| ATOM | 29 | C41 | MOL | 3 | 2.880 | 19.515 | 17.181 | −0.20 | +0.05 | +0.113 | 2.639 |
| ATOM | 30 | O48 | MOL | 3 | 4.000 | 20.163 | 17.734 | +0.11 | −0.40 | −0.537 | 2.639 |
| ATOM | 31 | H48 | MOL | 3 | 3.727 | 20.967 | 18.150 | +0.10 | +0.29 | +0.424 | 2.639 |
| ATOM | 32 | C42 | MOL | 3 | 2.245 | 20.486 | 16.201 | −0.32 | +0.06 | +0.113 | 2.639 |
| ATOM | 33 | O46 | MOL | 3 | 1.835 | 21.656 | 16.841 | +0.01 | −0.14 | −0.537 | 2.639 |
| ATOM | 34 | H46 | MOL | 3 | 1.008 | 21.471 | 17.268 | −0.36 | −0.25 | +0.424 | 2.639 |
| ATOM | 35 | C43 | MOL | 3 | 1.010 | 19.846 | 15.568 | −0.43 | +0.12 | +0.211 | 2.639 |
| ATOM | 36 | N51 | MOL | 3 | 0.483 | 20.623 | 14.485 | −0.37 | −0.60 | −0.650 | 2.639 |
| ATOM | 37 | H51 | MOL | 3 | 0.797 | 20.409 | 13.528 | +0.08 | +0.48 | +0.440 | 2.639 |
| ATOM | 38 | C53 | MOL | 3 | −0.412 | 21.628 | 14.664 | −0.77 | +0.37 | +0.396 | 2.639 |
| ATOM | 39 | O54 | MOL | 3 | −0.949 | 21.856 | 15.711 | −0.31 | −0.24 | −0.396 | 2.639 |
| ATOM | 40 | C55 | MOL | 3 | −0.661 | 22.468 | 13.431 | −0.52 | +0.00 | +0.000 | 2.639 |
| ATOM | 41 | C18 | MOL | 3 | 0.458 | 16.152 | 8.433 | −0.67 | +0.07 | +0.113 | 2.639 |
| ATOM | 42 | O23 | MOL | 3 | −0.489 | 15.121 | 8.141 | −0.28 | −0.27 | −0.537 | 2.639 |
| ATOM | 43 | H23 | MOL | 3 | −0.582 | 15.019 | 7.119 | −0.13 | +0.10 | +0.424 | 2.639 |
| ATOM | 44 | C14 | MOL | 3 | 0.452 | 17.125 | 7.231 | −0.68 | +0.08 | +0.113 | 2.639 |
| ATOM | 45 | O16 | MOL | 3 | 0.211 | 16.414 | 6.040 | −0.26 | −0.34 | −0.537 | 2.639 |
| ATOM | 46 | H16 | MOL | 3 | 1.011 | 15.796 | 5.840 | −0.05 | +0.13 | +0.424 | 2.639 |
| TER | | | | | | | | | | | |
| ENDMDL | | | | | | | | | | | |
| MODEL | 15 | | | | | | | | | | |
| USER | Run = 15 | | | | | | | | | | |
| USER | Cluster Rank = 4 | | | | | | | | | | |
| USER | Number of conformations in this cluster = 1 | | | | | | | | | | |
| USER | | | | | | | | | | | |
| USER | RMSD from reference structure = 2.357 A | | | | | | | | | | |
| USER | | | | | | | | | | | |
| USER | Estimated Free Energy of Binding = −11.14 kcal/mol [= (1) + (3)] | | | | | | | | | | |
| USER | Estimated Inhibition Constant, Ki = +6.83e−09 [Temperature = 298.15 K] | | | | | | | | | | |
| USER | | | | | | | | | | | |
| USER | Final Docked Energy = −14.27 kcal/mol [= (1) + (2)] | | | | | | | | | | |
| USER | | | | | | | | | | | |
| USER | (1) Final Intermolecular Energy = −15.50 kcal/mol | | | | | | | | | | |
| USER | (2) Final Internal Energy of Ligand = +1.23 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −1.885183 18.542385 13.095416 | | | | | | | | | | |
| USER | NEWDPF quat0 −0.244916 −0.731261 −0.636611 26.291954 | | | | | | | | | | |
| USER | NEWDPF ndihe 14 | | | | | | | | | | |
| USER | NEWDPF dihe0 −80.30 103.41 90.05 −73.90 69.78 −148.03 −11.12 −57.83 −140.70 | | | | | | | | | | |
| | −113.73 44.74 −113.80 110.17 178.98 | | | | | | | | | | |
| USER | | | | | | | | | | | |
| USER | | | | Rank | x | y | z | vdW | Elec | q | RMS |
| ATOM | 1 | N1 | MOL | 4 | −0.839 | 19.463 | 6.890 | −0.37 | −0.20 | −0.211 | 2.357 |
| ATOM | 2 | C8 | MOL | 4 | 0.281 | 20.286 | 6.893 | −0.54 | +0.00 | +0.000 | 2.357 |
| ATOM | 3 | C6 | MOL | 4 | 0.351 | 21.430 | 6.196 | −0.55 | +0.00 | +0.000 | 2.357 |
| ATOM | 4 | C5 | MOL | 4 | −0.753 | 21.826 | 5.378 | −0.64 | +0.25 | +0.396 | 2.357 |
| ATOM | 5 | O11 | MOL | 4 | −0.842 | 22.841 | 4.688 | −0.11 | −0.27 | −0.396 | 2.357 |
| ATOM | 6 | N3 | MOL | 4 | −1.840 | 20.952 | 5.409 | −0.47 | −0.22 | −0.440 | 2.357 |
| ATOM | 7 | H3 | MOL | 4 | −2.660 | 21.211 | 4.841 | −0.26 | −0.16 | +0.440 | 2.357 |
| ATOM | 8 | C2 | MOL | 4 | −1.924 | 19.784 | 6.122 | −0.80 | +0.38 | +0.396 | 2.357 |
| ATOM | 9 | O10 | MOL | 4 | −2.927 | 19.070 | 6.086 | −0.43 | −0.74 | −0.396 | 2.357 |
| ATOM | 10 | C12 | MOL | 4 | −0.868 | 18.233 | 7.693 | −0.63 | +0.32 | +0.324 | 2.357 |
| ATOM | 11 | O22 | MOL | 4 | −0.784 | 18.641 | 9.053 | −0.08 | −0.23 | −0.227 | 2.357 |
| ATOM | 12 | C20 | MOL | 4 | −0.285 | 17.537 | 9.855 | −0.64 | +0.11 | +0.113 | 2.357 |
| ATOM | 13 | C25 | MOL | 4 | 0.750 | 18.078 | 10.843 | −0.47 | +0.12 | +0.113 | 2.357 |
| ATOM | 14 | O28 | MOL | 4 | 1.788 | 18.682 | 10.095 | +0.00 | −0.41 | −0.368 | 2.357 |
| ATOM | 15 | P29 | MOL | 4 | 3.179 | 18.165 | 9.561 | −0.52 | +1.51 | +1.019 | 2.357 |
| ATOM | 16 | O30 | MOL | 4 | 3.397 | 16.797 | 10.110 | −0.48 | −0.48 | −0.255 | 2.357 |
| ATOM | 17 | O31 | MOL | 4 | 3.218 | 18.349 | 8.075 | −0.08 | −0.33 | −0.255 | 2.357 |
| ATOM | 18 | O32 | MOL | 4 | 4.059 | 19.219 | 10.374 | −0.30 | −0.90 | −0.510 | 2.357 |
| ATOM | 19 | P33 | MOL | 4 | 4.034 | 19.725 | 11.891 | −0.53 | +1.41 | +1.019 | 2.357 |
| ATOM | 20 | O35 | MOL | 4 | 2.797 | 20.548 | 11.891 | −0.04 | −0.31 | −0.255 | 2.357 |
| ATOM | 21 | O36 | MOL | 4 | 5.316 | 20.298 | 12.371 | −0.27 | −0.36 | −0.255 | 2.357 |
| ATOM | 22 | O34 | MOL | 4 | 3.849 | 18.377 | 12.770 | +0.05 | −0.48 | −0.368 | 2.357 |
| ATOM | 23 | C37 | MOL | 4 | 3.591 | 19.027 | 15.377 | −0.24 | +0.19 | +0.227 | 2.357 |
| ATOM | 24 | O39 | MOL | 4 | 2.742 | 19.879 | 15.024 | +0.10 | −0.18 | −0.227 | 2.357 |
| ATOM | 25 | C40 | MOL | 4 | 1.426 | 19.542 | 14.529 | −0.42 | +0.11 | +0.113 | 2.357 |
| ATOM | 26 | C50 | MOL | 4 | 0.666 | 20.854 | 14.577 | −0.58 | +0.10 | +0.113 | 2.357 |
| ATOM | 27 | O61 | MOL | 4 | −0.629 | 20.701 | 14.112 | −0.22 | −0.65 | −0.537 | 2.357 |
| ATOM | 28 | H61 | MOL | 4 | −1.190 | 20.496 | 14.875 | +0.06 | +0.50 | +0.424 | 2.357 |
| ATOM | 29 | C41 | MOL | 4 | 0.858 | 18.450 | 15.419 | −0.47 | +0.10 | +0.113 | 2.357 |
| ATOM | 30 | O48 | MOL | 4 | −0.369 | 17.975 | 14.918 | −0.22 | −0.81 | −0.537 | 2.357 |
| ATOM | 31 | H48 | MOL | 4 | −0.707 | 18.586 | 14.280 | +0.03 | +0.75 | +0.424 | 2.357 |
| ATOM | 32 | C42 | MOL | 4 | 1.813 | 17.269 | 15.443 | −0.39 | +0.10 | +0.113 | 2.357 |
| ATOM | 33 | O46 | MOL | 4 | 1.338 | 16.250 | 16.269 | −0.05 | −0.47 | −0.537 | 2.357 |
| ATOM | 34 | H46 | MOL | 4 | 2.092 | 15.778 | 16.601 | +0.08 | +0.37 | +0.424 | 2.357 |
| ATOM | 35 | C43 | MOL | 4 | 3.170 | 17.714 | 15.988 | −0.30 | +0.17 | +0.211 | 2.357 |
| ATOM | 36 | N51 | MOL | 4 | 4.177 | 16.704 | 15.846 | −0.22 | −0.58 | −0.650 | 2.357 |
| ATOM | 37 | H51 | MOL | 4 | 4.699 | 16.409 | 16.683 | +0.08 | +0.40 | +0.440 | 2.357 |
| ATOM | 38 | C53 | MOL | 4 | 4.474 | 16.120 | 14.658 | −0.42 | +0.40 | +0.396 | 2.357 |
| ATOM | 39 | O54 | MOL | 4 | 3.829 | 16.283 | 13.661 | −0.12 | −0.44 | −0.396 | 2.357 |
| ATOM | 40 | C53 | MOL | 4 | 5.716 | 15.257 | 14.680 | −0.61 | +0.00 | +0.000 | 2.357 |
| ATOM | 41 | C18 | MOL | 4 | 0.307 | 16.542 | 8.856 | −0.70 | +0.09 | +0.113 | 2.357 |
| ATOM | 42 | O23 | MOL | 4 | −0.610 | 15.463 | 8.651 | −0.29 | −0.38 | −0.537 | 2.357 |
| ATOM | 43 | H23 | MOL | 4 | −0.373 | 14.973 | 7.775 | −0.16 | +0.13 | +0.424 | 2.357 |
| ATOM | 44 | C14 | MOL | 4 | 0.367 | 17.341 | 7.533 | −0.63 | +0.09 | +0.113 | 2.357 |
| ATOM | 45 | O16 | MOL | 4 | 0.208 | 16.470 | 6.439 | −0.25 | −0.33 | −0.537 | 2.357 |
| ATOM | 46 | H16 | MOL | 4 | 0.925 | 15.732 | 6.480 | −0.11 | +0.03 | +0.424 | 2.357 |
| TER | | | | | | | | | | | |
| ENDMDL | | | | | | | | | | | |
| MODEL | 82 | | | | | | | | | | |
| USER | Run = 82 | | | | | | | | | | |
| USER | Cluster Rank = 5 | | | | | | | | | | |
| USER | Number of conformations in this cluster = 1 | | | | | | | | | | |
| USER | | | | | | | | | | | |
| USER | RMSD from reference structure = 1.930 A | | | | | | | | | | |
| USER | | | | | | | | | | | |
| USER | Estimated Free Energy of Binding = −10.21 kcal/mol [= (1) + (3)] | | | | | | | | | | |
| USER | Estimated Inhibition Constant, Ki = +3.29e−08 [Temperature = 298.15 K] | | | | | | | | | | |
| USER | | | | | | | | | | | |
| USER | Final Docked Energy = −14.04 kcal/mol [= (1) + (2)] | | | | | | | | | | |
| USER | | | | | | | | | | | |
| USER | (1) Final Intermolecular Energy = −14.57 kcal/mol | | | | | | | | | | |
| USER | (2) Final Internal Energy of Ligand = +0.53 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 0.193903 17.421421 13.547938 | | | | | | | | | | |
| USER | NEWDPF quat0 −0.008747 −0.059915 −0.998165 18.282000 | | | | | | | | | | |
| USER | NEWDPF ndihe 14 | | | | | | | | | | |
| USER | NEWDPF dihe0 13.49 37.08 −171.27 −65.94 93.80 −171.40 161.25 −47.65 −75.60 | | | | | | | | | | |
| | −32.21 −150.60 163.10 54.42 −74.79 | | | | | | | | | | |
| USER | | | | | | | | | | | |
| USER | | | | Rank | x | y | z | vdW | Elec | q | RMS |
| ATOM | 1 | N1 | MOL | 5 | −0.543 | 19.314 | 7.520 | −0.34 | −0.20 | −0.211 | 1.930 |
| ATOM | 2 | C8 | MOL | 5 | 0.567 | 20.127 | 7.324 | −0.46 | +0.00 | +0.000 | 1.930 |
| ATOM | 3 | C6 | MOL | 5 | 0.486 | 21.367 | 6.818 | −0.34 | +0.00 | +0.000 | 1.930 |
| ATOM | 4 | C5 | MOL | 5 | −0.788 | 21.886 | 6.426 | −0.70 | +0.30 | +0.396 | 1.930 |
| ATOM | 5 | O11 | MOL | 5 | −1.026 | 22.998 | 5.954 | −0.13 | −0.31 | −0.396 | 1.930 |
| ATOM | 6 | N3 | MOL | 5 | −1.859 | 21.018 | 6.638 | −0.51 | −0.34 | −0.440 | 1.930 |
| ATOM | 7 | H3 | MOL | 5 | −2.794 | 21.362 | 6.379 | −0.04 | +0.24 | +0.440 | 1.930 |
| ATOM | 8 | C2 | MOL | 5 | −1.787 | 19.752 | 7.159 | −0.74 | +0.42 | +0.396 | 1.930 |
| ATOM | 9 | O10 | MOL | 5 | −2.789 | 19.051 | 7.308 | −0.50 | −0.63 | −0.396 | 1.930 |
| ATOM | 10 | C12 | MOL | 5 | −0.396 | 17.972 | 8.101 | −0.60 | +0.30 | +0.324 | 1.930 |
| ATOM | 11 | O22 | MOL | 5 | 0.093 | 18.160 | 9.424 | −0.05 | −0.22 | −0.227 | 1.930 |
| ATOM | 12 | C20 | MOL | 5 | 0.751 | 16.943 | 9.869 | −0.56 | +0.10 | +0.113 | 1.930 |
| ATOM | 13 | C25 | MOL | 5 | 2.049 | 17.322 | 10.584 | −0.51 | +0.13 | +0.113 | 1.930 |
| ATOM | 14 | O28 | MOL | 5 | 1.755 | 18.368 | 11.490 | +0.00 | −0.43 | −0.368 | 1.930 |
| ATOM | 15 | P29 | MOL | 5 | 2.594 | 19.577 | 12.057 | −0.43 | +1.20 | +1.019 | 1.930 |
| ATOM | 16 | O30 | MOL | 5 | 3.351 | 20.177 | 10.923 | −0.11 | −0.38 | −0.255 | 1.930 |
| ATOM | 17 | O31 | MOL | 5 | 1.682 | 20.462 | 12.849 | +0.09 | −0.28 | −0.255 | 1.930 |
| ATOM | 18 | O32 | MOL | 5 | 3.572 | 18.683 | 12.946 | +0.09 | −0.60 | −0.510 | 1.930 |
| ATOM | 19 | P33 | MOL | 5 | 4.346 | 18.944 | 14.321 | −0.27 | +0.99 | +1.019 | 1.930 |
| ATOM | 20 | O35 | MOL | 5 | 3.734 | 20.229 | 14.747 | +0.08 | −0.21 | −0.255 | 1.930 |
| ATOM | 21 | O36 | MOL | 5 | 5.821 | 18.809 | 14.233 | +0.07 | −0.26 | −0.255 | 1.930 |
| ATOM | 22 | O34 | MOL | 5 | 3.893 | 17.724 | 15.284 | +0.12 | −0.33 | −0.368 | 1.930 |
| ATOM | 23 | C37 | MOL | 5 | 2.319 | 18.670 | 17.263 | −0.09 | +0.09 | +0.227 | 1.930 |
| ATOM | 24 | O39 | MOL | 5 | 1.548 | 19.095 | 16.370 | +0.08 | −0.09 | −0.227 | 1.930 |
| ATOM | 25 | C40 | MOL | 5 | 0.732 | 18.239 | 15.540 | −0.49 | +0.10 | +0.113 | 1.930 |
| ATOM | 26 | C50 | MOL | 5 | −0.259 | 19.188 | 14.891 | −0.60 | +0.13 | +0.113 | 1.930 |
| ATOM | 27 | O61 | MOL | 5 | −0.745 | 18.670 | 13.704 | −0.40 | −1.04 | −0.537 | 1.930 |
| ATOM | 28 | H61 | MOL | 5 | −1.303 | 19.351 | 13.296 | +0.04 | +0.90 | +0.424 | 1.930 |
| ATOM | 29 | C41 | MOL | 5 | 0.104 | 17.183 | 16.433 | −0.62 | +0.10 | +0.113 | 1.930 |
| ATOM | 30 | O48 | MOL | 5 | −0.600 | 16.231 | 15.673 | −0.17 | −0.65 | −0.537 | 1.930 |
| ATOM | 31 | H48 | MOL | 5 | −1.460 | 16.565 | 15.464 | +0.02 | +0.63 | +0.424 | 1.930 |
| ATOM | 32 | C42 | MOL | 5 | 1.196 | 16.436 | 17.179 | −0.56 | +0.09 | +0.113 | 1.930 |
| ATOM | 33 | O46 | MOL | 5 | 0.659 | 15.469 | 18.029 | −0.30 | −0.61 | −0.537 | 1.930 |
| ATOM | 34 | H46 | MOL | 5 | 0.015 | 15.900 | 18.578 | +0.03 | +0.42 | +0.424 | 1.930 |
| ATOM | 35 | C43 | MOL | 5 | 1.997 | 17.415 | 18.036 | −0.45 | +0.14 | +0.211 | 1.930 |
| ATOM | 36 | N51 | MOL | 5 | 3.169 | 16.816 | 18.604 | −0.30 | −0.59 | −0.650 | 1.930 |
| ATOM | 37 | H51 | MOL | 5 | 3.777 | 16.251 | 17.994 | +0.07 | +0.40 | +0.440 | 1.930 |
| ATOM | 38 | C53 | MOL | 5 | 3.514 | 16.959 | 19.909 | −0.60 | +0.48 | +0.396 | 1.930 |
| ATOM | 39 | O54 | MOL | 5 | 2.960 | 17.712 | 20.660 | −0.42 | −0.71 | −0.396 | 1.930 |
| ATOM | 40 | C55 | MOL | 5 | 4.647 | 16.064 | 20.357 | −0.61 | +0.00 | +0.000 | 1.930 |
| ATOM | 41 | C18 | MOL | 5 | 0.985 | 16.119 | 8.602 | −0.50 | +0.07 | +0.113 | 1.930 |
| ATOM | 42 | O23 | MOL | 5 | 0.001 | 15.086 | 8.511 | −0.37 | −0.26 | −0.537 | 1.930 |
| ATOM | 43 | H23 | MOL | 5 | 0.047 | 14.650 | 7.578 | −0.34 | −0.05 | +0.424 | 1.930 |
| ATOM | 44 | C14 | MOL | 5 | 0.700 | 17.118 | 7.457 | −0.65 | +0.08 | +0.113 | 1.930 |
| ATOM | 45 | O16 | MOL | 5 | 0.195 | 16.431 | 6.336 | −0.26 | −0.33 | −0.537 | 1.930 |
| ATOM | 46 | H16 | MOL | 5 | 0.609 | 15.489 | 6.295 | −0.08 | +0.04 | +0.424 | 1.930 |
| TER | | | | | | | | | | | |
| ENDMDL | | | | | | | | | | | |
| MODEL | 67 | | | | | | | | | | |
| USER | Run = 67 | | | | | | | | | | |
| USER | Cluster Rank = 6 | | | | | | | | | | |
| USER | Number of conformations in this cluster = 1 | | | | | | | | | | |
| USER | | | | | | | | | | | |
| USER | RMSD from reference structure = 2.315 A | | | | | | | | | | |
| USER | | | | | | | | | | | |
| USER | Estimated Free Energy of Binding = −10.51 kcal/mol [= (1) + (3)] | | | | | | | | | | |
| USER | Estimated Inhibition Constant, Ki = +1.98e−08 [Temperature = 298.15 K] | | | | | | | | | | |
| USER | | | | | | | | | | | |
| USER | Final Docked Energy = −14.02 kcal/mol [= (1) + (2)] | | | | | | | | | | |
| USER | | | | | | | | | | | |
| USER | (1) Final Intermolecular Energy = −14.87 kcal/mol | | | | | | | | | | |
| USER | (2) Final Internal Energy of Ligand = +0.85 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −1.841907 18.296664 12.801292 | | | | | | | | | | |
| USER | NEWDPF quat0 0.212135 0.883799 0.417012 −24.189452 | | | | | | | | | | |
| USER | NEWDPF ndihe 14 | | | | | | | | | | |
| USER | NEWDPF dihe0 −173.02 −25.87 34.48 −36.19 −123.99 17.62 157.83 −14.42 −143.56 | | | | | | | | | | |
| | 164.52 86.74 123.61 90.14 176.84 | | | | | | | | | | |
| USER | | | | | | | | | | | |
| USER | | | | Rank | x | y | z | vdW | Elec | q | RMS |
| ATOM | 1 | N1 | MOL | 6 | −0.712 | 19.333 | 6.628 | −0.37 | −0.19 | −0.211 | 2.315 |
| ATOM | 2 | C8 | MOL | 6 | 0.302 | 20.283 | 6.665 | −0.54 | +0.00 | +0.000 | 2.315 |
| ATOM | 3 | C6 | MOL | 6 | 0.258 | 21.427 | 5.965 | −0.56 | +0.00 | +0.000 | 2.315 |
| ATOM | 4 | C5 | MOL | 6 | −0.858 | 21.689 | 5.111 | −0.58 | +0.23 | +0.396 | 2.315 |
| ATOM | 5 | O11 | MOL | 6 | −1.045 | 22.686 | 4.414 | −0.11 | −0.24 | −0.396 | 2.315 |
| ATOM | 6 | N3 | MOL | 6 | −1.834 | 20.693 | 5.110 | −0.42 | −0.20 | −0.440 | 2.315 |
| ATOM | 7 | H3 | MOL | 6 | −2.660 | 20.852 | 4.516 | −0.26 | −0.19 | +0.440 | 2.315 |
| ATOM | 8 | C2 | MOL | 6 | −1.802 | 19.523 | 5.825 | −0.76 | +0.38 | +0.396 | 2.315 |
| ATOM | 9 | O10 | MOL | 6 | −2.712 | 18.695 | 5.760 | −0.43 | −0.70 | −0.396 | 2.315 |
| ATOM | 10 | C12 | MOL | 6 | −0.621 | 18.108 | 7.435 | −0.63 | +0.31 | +0.324 | 2.315 |
| ATOM | 11 | O22 | MOL | 6 | −0.630 | 18.524 | 8.796 | −0.06 | −0.22 | −0.227 | 2.315 |
| ATOM | 12 | C20 | MOL | 6 | −0.030 | 17.487 | 9.618 | −0.63 | +0.10 | +0.113 | 2.315 |
| ATOM | 13 | C25 | MOL | 6 | 0.901 | 18.148 | 10.636 | −0.51 | +0.12 | +0.113 | 2.315 |
| ATOM | 14 | O28 | MOL | 6 | 1.913 | 18.829 | 9.920 | +0.00 | −0.42 | −0.368 | 2.315 |
| ATOM | 15 | P29 | MOL | 6 | 3.473 | 18.631 | 9.796 | −0.56 | +1.60 | +1.019 | 2.315 |
| ATOM | 16 | O30 | MOL | 6 | 3.839 | 17.411 | 10.571 | −0.54 | −0.50 | −0.255 | 2.315 |
| ATOM | 17 | O31 | MOL | 6 | 3.848 | 18.691 | 8.348 | −0.26 | −0.41 | −0.255 | 2.315 |
| ATOM | 18 | O32 | MOL | 6 | 3.862 | 19.935 | 10.629 | −0.25 | −0.87 | −0.510 | 2.315 |
| ATOM | 19 | P33 | MOL | 6 | 4.085 | 20.212 | 12.188 | −0.53 | +1.36 | +1.019 | 2.315 |
| ATOM | 20 | O35 | MOL | 6 | 3.763 | 21.659 | 12.265 | −0.31 | −0.33 | −0.255 | 2.315 |
| ATOM | 21 | O36 | MOL | 6 | 5.369 | 19.699 | 12.727 | −0.08 | −0.31 | −0.255 | 2.315 |
| ATOM | 22 | O34 | MOL | 6 | 2.958 | 19.314 | 12.928 | +0.07 | −0.41 | −0.368 | 2.315 |
| ATOM | 23 | C37 | MOL | 6 | 3.118 | 19.684 | 15.597 | −0.27 | +0.17 | +0.227 | 2.315 |
| ATOM | 24 | O39 | MOL | 6 | 2.275 | 20.602 | 15.464 | +0.05 | −0.16 | −0.227 | 2.315 |
| ATOM | 25 | C40 | MOL | 6 | 0.885 | 20.374 | 15.141 | −0.51 | +0.08 | +0.113 | 2.315 |
| ATOM | 26 | C50 | MOL | 6 | 0.214 | 21.702 | 15.437 | −0.64 | +0.07 | +0.113 | 2.315 |
| ATOM | 27 | O61 | MOL | 6 | 1.031 | 22.518 | 16.199 | −0.10 | −0.29 | −0.537 | 2.315 |
| ATOM | 28 | H61 | MOL | 6 | 1.847 | 22.026 | 16.380 | +0.07 | +0.23 | +0.424 | 2.315 |
| ATOM | 29 | C41 | MOL | 6 | 0.392 | 19.214 | 15.989 | −0.31 | +0.05 | +0.113 | 2.315 |
| ATOM | 30 | O48 | MOL | 6 | −0.919 | 18.847 | 15.633 | +0.01 | −0.48 | −0.537 | 2.315 |
| ATOM | 31 | H48 | MOL | 6 | −1.390 | 18.570 | 16.405 | +0.07 | +0.23 | +0.424 | 2.315 |
| ATOM | 32 | C42 | MOL | 6 | 1.273 | 18.001 | 15.749 | −0.43 | +0.09 | +0.113 | 2.315 |
| ATOM | 33 | O46 | MOL | 6 | 0.867 | 16.917 | 16.528 | −0.06 | −0.44 | −0.537 | 2.315 |
| ATOM | 34 | H46 | MOL | 6 | 1.124 | 16.126 | 16.071 | +0.06 | +0.39 | +0.424 | 2.315 |
| ATOM | 35 | C43 | MOL | 6 | 2.717 | 18.329 | 16.127 | −0.29 | +0.15 | +0.211 | 2.315 |
| ATOM | 36 | N51 | MOL | 6 | 3.635 | 17.302 | 15.732 | −0.18 | −0.56 | −0.650 | 2.315 |
| ATOM | 37 | H51 | MOL | 6 | 3.530 | 16.882 | 14.798 | +0.09 | +0.40 | +0.440 | 2.315 |
| ATOM | 38 | C53 | MOL | 6 | 4.637 | 16.858 | 16.534 | −0.40 | +0.36 | +0.396 | 2.315 |
| ATOM | 39 | O54 | MOL | 6 | 4.934 | 17.380 | 17.571 | +0.02 | −0.38 | −0.396 | 2.315 |
| ATOM | 40 | C55 | MOL | 6 | 5.340 | 15.619 | 16.025 | −0.45 | +0.00 | +0.000 | 2.315 |
| ATOM | 41 | C18 | MOL | 6 | 0.708 | 16.569 | 8.642 | −0.67 | +0.08 | +0.113 | 2.315 |
| ATOM | 42 | O23 | MOL | 6 | −0.068 | 15.389 | 8.413 | −0.32 | −0.28 | −0.537 | 2.315 |
| ATOM | 43 | H23 | MOL | 6 | 0.540 | 14.648 | 8.030 | −0.28 | −0.11 | +0.424 | 2.315 |
| ATOM | 44 | C14 | MOL | 6 | 0.715 | 17.370 | 7.319 | −0.63 | +0.09 | +0.113 | 2.315 |
| ATOM | 45 | O16 | MOL | 6 | 0.696 | 16.485 | 6.224 | −0.23 | −0.31 | −0.537 | 2.315 |
| ATOM | 46 | H16 | MOL | 6 | −0.218 | 16.541 | 5.752 | +0.03 | +0.32 | +0.424 | 2.315 |
| TER | | | | | | | | | | | |
| ENDMDL | | | | | | | | | | | |
| MODEL | 55 | | | | | | | | | | |
| USER | Run = 55 | | | | | | | | | | |
| USER | Cluster Rank = 7 | | | | | | | | | | |
| USER | Number of conformations in this cluster = 1 | | | | | | | | | | |
| USER | | | | | | | | | | | |
| USER | RMSD from reference structure = 2.537 A | | | | | | | | | | |
| USER | | | | | | | | | | | |
| USER | Estimated Free Energy of Binding = −10.49 kcal/mol [= (1) + (3)] | | | | | | | | | | |
| USER | Estimated Inhibition Constant, Ki = +2.06e−08 [Temperature = 298.15 K] | | | | | | | | | | |
| USER | | | | | | | | | | | |
| USER | Final Docked Energy = −13.98 kcal/mol [= (1) + (2)] | | | | | | | | | | |
| USER | | | | | | | | | | | |
| USER | (1) Final Intermolecular Energy = −14.84 kcal/mol | | | | | | | | | | |
| USER | (2) Final Internal Energy of Ligand = +0.86 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −0.652910 18.021718 13.498063 | | | | | | | | | | |
| USER | NEWDPF quat0 −0.275211 −0.299395 −0.913576 22.617879 | | | | | | | | | | |
| USER | NEWDPF ndihe 14 | | | | | | | | | | |
| USER | NEWDPF dihe0 30.21 5.52 45.90 −49.02 −170.17 90.62 173.88 55.56 −57.61 | | | | | | | | | | |
| | 176.17 93.35 −123.78 −37.83 160.53 | | | | | | | | | | |
| USER | | | | | | | | | | | |
| USER | | | | Rank | x | y | z | vdW | Elec | q | RMS |
| ATOM | 1 | N1 | MOL | 7 | −0.826 | 19.201 | 7.250 | −0.37 | −0.20 | −0.211 | 2.537 |
| ATOM | 2 | C8 | MOL | 7 | 0.333 | 19.942 | 7.053 | −0.52 | +0.00 | +0.000 | 2.537 |
| ATOM | 3 | C6 | MOL | 7 | 0.352 | 21.114 | 6.401 | −0.54 | +0.00 | +0.000 | 2.537 |
| ATOM | 4 | C5 | MOL | 7 | −0.860 | 21.631 | 5.846 | −0.69 | +0.27 | +0.396 | 2.537 |
| ATOM | 5 | O11 | MOL | 7 | −1.007 | 22.686 | 5.229 | −0.15 | −0.27 | −0.396 | 2.537 |
| ATOM | 6 | N3 | MOL | 7 | −1.985 | 20.836 | 6.069 | −0.53 | −0.30 | −0.440 | 2.537 |
| ATOM | 7 | H3 | MOL | 7 | −2.880 | 21.183 | 5.694 | −0.24 | +0.08 | +0.440 | 2.537 |
| ATOM | 8 | C2 | MOL | 7 | −2.015 | 19.640 | 6.738 | −0.80 | +0.43 | +0.396 | 2.537 |
| ATOM | 9 | O10 | MOL | 7 | −3.059 | 19.002 | 6.884 | −0.46 | −0.80 | −0.396 | 2.537 |
| ATOM | 10 | C12 | MOL | 7 | −0.789 | 17.935 | 7.994 | −0.63 | +0.31 | +0.324 | 2.537 |
| ATOM | 11 | O22 | MOL | 7 | −0.396 | 18.262 | 9.322 | −0.07 | −0.22 | −0.227 | 2.537 |
| ATOM | 12 | C20 | MOL | 7 | 0.165 | 17.082 | 9.960 | −0.56 | +0.10 | +0.113 | 2.537 |
| ATOM | 13 | C25 | MOL | 7 | 1.420 | 17.495 | 10.730 | −0.45 | +0.12 | +0.113 | 2.537 |
| ATOM | 14 | O28 | MOL | 7 | 2.470 | 17.655 | 9.796 | −0.06 | −0.46 | −0.368 | 2.537 |
| ATOM | 15 | P29 | MOL | 7 | 3.355 | 18.886 | 9.362 | −0.51 | +1.58 | +1.019 | 2.537 |
| ATOM | 16 | O30 | MOL | 7 | 4.767 | 18.583 | 9.733 | −0.49 | −0.54 | −0.255 | 2.537 |
| ATOM | 17 | O31 | MOL | 7 | 3.051 | 19.214 | 7.934 | −0.05 | −0.33 | −0.255 | 2.537 |
| ATOM | 18 | O32 | MOL | 7 | 2.728 | 19.906 | 10.417 | −0.02 | −0.68 | −0.510 | 2.537 |
| ATOM | 19 | P33 | MOL | 7 | 3.162 | 20.365 | 11.887 | −0.44 | +1.28 | +1.019 | 2.537 |
| ATOM | 20 | O35 | MOL | 7 | 1.837 | 20.480 | 12.547 | +0.18 | −0.28 | −0.255 | 2.537 |
| ATOM | 21 | O36 | MOL | 7 | 4.119 | 21.499 | 11.921 | −0.20 | −0.43 | −0.255 | 2.537 |
| ATOM | 22 | O34 | MOL | 7 | 4.010 | 19.115 | 12.471 | +0.04 | −0.47 | −0.368 | 2.537 |
| ATOM | 23 | C37 | MOL | 7 | 2.364 | 17.470 | 13.841 | −0.14 | +0.23 | +0.227 | 2.537 |
| ATOM | 24 | O39 | MOL | 7 | 3.244 | 16.584 | 13.725 | +0.08 | −0.21 | −0.227 | 2.537 |
| ATOM | 25 | C40 | MOL | 7 | 4.508 | 16.613 | 14.424 | −0.37 | +0.12 | +0.113 | 2.537 |
| ATOM | 26 | C50 | MOL | 7 | 5.057 | 15.208 | 14.254 | −0.60 | +0.11 | +0.113 | 2.537 |
| ATOM | 27 | O61 | MOL | 7 | 5.184 | 14.879 | 12.916 | −0.39 | −0.67 | −0.537 | 2.537 |
| ATOM | 28 | H61 | MOL | 7 | 5.190 | 13.911 | 12.854 | −0.24 | +0.10 | +0.424 | 2.537 |
| ATOM | 29 | C41 | MOL | 7 | 4.237 | 17.015 | 15.864 | −0.34 | +0.10 | +0.113 | 2.537 |
| ATOM | 30 | O48 | MOL | 7 | 5.438 | 17.194 | 16.575 | +0.08 | −0.52 | −0.537 | 2.537 |
| ATOM | 31 | H48 | MOL | 7 | 5.509 | 16.531 | 17.246 | +0.07 | +0.41 | +0.424 | 2.537 |
| ATOM | 32 | C42 | MOL | 7 | 3.505 | 18.345 | 15.888 | −0.25 | +0.09 | +0.113 | 2.537 |
| ATOM | 33 | O46 | MOL | 7 | 3.216 | 18.741 | 17.195 | +0.10 | −0.35 | −0.537 | 2.537 |
| ATOM | 34 | H46 | MOL | 7 | 2.993 | 19.663 | 17.170 | +0.07 | +0.21 | +0.424 | 2.537 |
| ATOM | 35 | C43 | MOL | 7 | 2.179 | 18.218 | 15.138 | −0.33 | +0.19 | +0.211 | 2.537 |
| ATOM | 36 | N51 | MOL | 7 | 1.535 | 19.484 | 14.943 | −0.22 | −0.54 | −0.650 | 2.537 |
| ATOM | 37 | H51 | MOL | 7 | 2.036 | 20.215 | 14.420 | +0.10 | +0.39 | +0.440 | 2.537 |
| ATOM | 38 | C53 | MOL | 7 | 0.294 | 19.762 | 15.417 | −0.54 | +0.27 | +0.396 | 2.537 |
| ATOM | 39 | O54 | MOL | 7 | −0.427 | 18.938 | 15.906 | −0.15 | −0.26 | −0.396 | 2.537 |
| ATOM | 40 | C55 | MOL | 7 | −0.104 | 21.217 | 15.309 | −0.68 | +0.00 | +0.000 | 2.537 |
| ATOM | 41 | C18 | MOL | 7 | 0.457 | 16.104 | 8.821 | −0.72 | +0.08 | +0.113 | 2.537 |
| ATOM | 42 | O23 | MOL | 7 | −0.566 | 15.106 | 8.770 | −0.37 | −0.37 | −0.537 | 2.537 |
| ATOM | 43 | H23 | MOL | 7 | −0.313 | 14.390 | 8.072 | −0.33 | +0.17 | +0.424 | 2.537 |
| ATOM | 44 | C14 | MOL | 7 | 0.310 | 16.968 | 7.547 | −0.68 | +0.08 | +0.113 | 2.537 |
| ATOM | 45 | O16 | MOL | 7 | −0.139 | 16.171 | 6.477 | −0.28 | −0.32 | −0.537 | 2.537 |
| ATOM | 46 | H16 | MOL | 7 | −1.080 | 15.809 | 6.689 | +0.02 | +0.30 | +0.424 | 2.537 |
| TER | | | | | | | | | | | |
| ENDMDL | | | | | | | | | | | |
| MODEL | 72 | | | | | | | | | | |
| USER | Run = 72 | | | | | | | | | | |
| USER | Cluster Rank = 8 | | | | | | | | | | |
| USER | Number of conformations in this cluster = 2 | | | | | | | | | | |
| USER | | | | | | | | | | | |
| USER | RMSD from reference structure = 2.922 A | | | | | | | | | | |
| USER | | | | | | | | | | | |
| USER | Estimated Free Energy of Binding = −10.64 kcal/mol [= (1) + (3)] | | | | | | | | | | |
| USER | Estimated Inhibition Constant, Ki = +1.59e−08 [Temperature = 298.15 K] | | | | | | | | | | |
| USER | | | | | | | | | | | |
| USER | Final Docked Energy = −13.86 kcal/mol [= (1) + (2)] | | | | | | | | | | |
| USER | | | | | | | | | | | |
| USER | (1) Final Intermolecular Energy = −15.00 kcal/mol | | | | | | | | | | |
| USER | (2) Final Internal Energy of Ligand = +1.14 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −1.524094 17.653868 12.293702 | | | | | | | | | | |
| USER | NEWDPF quat0 0.070250 −0.728192 −0.681763 22.721636 | | | | | | | | | | |
| USER | NEWDPF ndihe 14 | | | | | | | | | | |
| USER | NEWDPF dihe0 100.39 −38.45 −111.32 30.63 106.78 127.39 175.81 93.21 −143.01 | | | | | | | | | | |
| | 31.02 122.18 −164.69 17.90 153.28 | | | | | | | | | | |
| USER | | | | | | | | | | | |
| USER | | | | Rank | x | y | z | vdW | Elec | q | RMS |
| ATOM | 1 | N1 | MOL | 8 | −0.707 | 19.470 | 6.253 | −0.37 | −0.19 | −0.211 | 2.922 |
| ATOM | 2 | C8 | MOL | 8 | 0.378 | 20.334 | 6.344 | −0.54 | +0.00 | +0.000 | 2.922 |
| ATOM | 3 | C6 | MOL | 8 | 0.380 | 21.565 | 5.811 | −0.55 | +0.00 | +0.000 | 2.922 |
| ATOM | 4 | C8 | MOL | 8 | −0.764 | 22.020 | 5.084 | −0.61 | +0.24 | +0.396 | 2.922 |
| ATOM | 5 | O11 | MOL | 8 | −0.916 | 23.116 | 4.545 | −0.10 | −0.28 | −0.396 | 2.922 |
| ATOM | 6 | N3 | MOL | 8 | −1.814 | 21.103 | 5.020 | −0.37 | −0.16 | −0.440 | 2.922 |
| ATOM | 7 | H3 | MOL | 8 | −2.660 | 21.401 | 4.514 | −0.23 | −0.30 | +0.440 | 2.922 |
| ATOM | 8 | C2 | MOL | 8 | −1.827 | 19.845 | 5.565 | −0.74 | +0.32 | +0.396 | 2.922 |
| ATOM | 9 | O10 | MOL | 8 | −2.801 | 19.099 | 5.454 | −0.41 | −0.56 | −0.396 | 2.922 |
| ATOM | 10 | C12 | MOL | 8 | −0.661 | 18.140 | 6.877 | −0.66 | +0.30 | +0.324 | 2.922 |
| ATOM | 11 | O22 | MOL | 8 | −0.552 | 18.360 | 8.279 | −0.04 | −0.22 | −0.227 | 2.922 |
| ATOM | 12 | C20 | MOL | 8 | 0.016 | 17.179 | 8.906 | −0.65 | +0.10 | +0.113 | 2.922 |
| ATOM | 13 | C25 | MOL | 8 | 1.057 | 17.624 | 9.934 | −0.55 | +0.11 | +0.113 | 2.922 |
| ATOM | 14 | O28 | MOL | 8 | 2.282 | 17.820 | 9.253 | −0.03 | −0.43 | −0.368 | 2.922 |
| ATOM | 15 | P29 | MOL | 8 | 3.685 | 18.393 | 9.691 | −0.56 | +1.70 | +1.019 | 2.922 |
| ATOM | 16 | O30 | MOL | 8 | 4.366 | 17.350 | 10.509 | −0.66 | −0.61 | −0.255 | 2.922 |
| ATOM | 17 | O31 | MOL | 8 | 4.388 | 18.914 | 8.477 | −0.32 | −0.52 | −0.255 | 2.922 |
| ATOM | 18 | O32 | MOL | 8 | 3.097 | 19.529 | 10.646 | −0.07 | −0.71 | −0.510 | 2.922 |
| ATOM | 19 | P33 | MOL | 8 | 3.309 | 19.875 | 12.193 | −0.50 | +1.24 | +1.019 | 2.922 |
| ATOM | 20 | O35 | MOL | 8 | 4.685 | 20.434 | 12.169 | −0.39 | −0.38 | −0.255 | 2.922 |
| ATOM | 21 | O36 | MOL | 8 | 2.965 | 18.771 | 13.124 | +0.09 | −0.28 | −0.255 | 2.922 |
| ATOM | 22 | O34 | MOL | 8 | 2.209 | 21.019 | 12.511 | +0.09 | −0.40 | −0.368 | 2.922 |
| ATOM | 23 | C37 | MOL | 8 | 2.454 | 21.818 | 15.078 | −0.22 | +0.21 | +0.227 | 2.922 |
| ATOM | 24 | O39 | MOL | 8 | 1.288 | 22.272 | 14.990 | −0.04 | −0.21 | −0.227 | 2.922 |
| ATOM | 25 | C40 | MOL | 8 | 0.108 | 21.441 | 14.905 | −0.70 | +0.09 | +0.113 | 2.922 |
| ATOM | 26 | C50 | MOL | 8 | −1.038 | 22.399 | 15.174 | −0.89 | +0.11 | +0.113 | 2.922 |
| ATOM | 27 | O61 | MOL | 8 | −1.571 | 22.886 | 13.993 | −0.40 | −0.71 | −0.537 | 2.922 |
| ATOM | 28 | H61 | MOL | 8 | −1.064 | 23.676 | 13.750 | +0.00 | +0.61 | +0.424 | 2.922 |
| ATOM | 29 | C41 | MOL | 8 | 0.250 | 20.323 | 15.924 | +0.00 | +0.03 | +0.113 | 2.922 |
| ATOM | 30 | O48 | MOL | 8 | −0.802 | 19.396 | 15.808 | −0.08 | −0.36 | −0.537 | 2.922 |
| ATOM | 31 | H48 | MOL | 8 | −0.901 | 18.926 | 16.623 | +0.04 | +0.13 | +0.424 | 2.922 |
| ATOM | 32 | C42 | MOL | 8 | 1.540 | 19.565 | 15.667 | −0.37 | +0.07 | +0.113 | 2.922 |
| ATOM | 33 | O46 | MOL | 8 | 1.720 | 18.540 | 16.597 | +0.06 | −0.25 | −0.537 | 2.922 |
| ATOM | 34 | H46 | MOL | 8 | 0.928 | 18.488 | 17.118 | −0.06 | +0.02 | +0.424 | 2.922 |
| ATOM | 35 | C43 | MOL | 8 | 2.731 | 20.516 | 15.788 | −0.34 | +0.14 | +0.211 | 2.922 |
| ATOM | 36 | N51 | MOL | 8 | 3.959 | 19.917 | 15.356 | −0.15 | −0.52 | −0.650 | 2.922 |
| ATOM | 37 | H51 | MOL | 8 | 4.035 | 19.602 | 14.378 | +0.10 | +0.40 | +0.440 | 2.922 |
| ATOM | 38 | C53 | MOL | 8 | 5.026 | 19.745 | 16.176 | −0.29 | +0.34 | +0.396 | 2.922 |
| ATOM | 39 | O54 | MOL | 8 | 5.092 | 20.207 | 17.280 | +0.09 | −0.35 | −0.396 | 2.922 |
| ATOM | 40 | C55 | MOL | 8 | 6.129 | 18.889 | 15.595 | −0.37 | +0.00 | +0.000 | 2.922 |
| ATOM | 41 | C18 | MOL | 8 | 0.616 | 16.359 | 7.763 | −0.58 | +0.06 | +0.113 | 2.922 |
| ATOM | 42 | O23 | MOL | 8 | −0.261 | 15.279 | 7.433 | −0.13 | −0.16 | −0.537 | 2.922 |
| ATOM | 43 | H23 | MOL | 8 | 0.027 | 14.433 | 7.947 | −0.39 | +0.07 | +0.424 | 2.922 |
| ATOM | 44 | C14 | MOL | 8 | 0.603 | 17.336 | 6.565 | −0.68 | +0.09 | +0.113 | 2.922 |
| ATOM | 45 | O16 | MOL | 8 | 0.447 | 16.618 | 5.363 | −0.02 | −0.41 | −0.537 | 2.922 |
| ATOM | 46 | H16 | MOL | 8 | 0.699 | 15.631 | 5.517 | −0.01 | +0.19 | +0.424 | 2.922 |
| TER | | | | | | | | | | | |
| ENDMDL | | | | | | | | | | | |
| MODEL | 97 | | | | | | | | | | |
| USER | Run = 97 | | | | | | | | | | |
| USER | Cluster Rank = 8 | | | | | | | | | | |
| USER | Number of conformations in this cluster = 2 | | | | | | | | | | |
| USER | | | | | | | | | | | |
| USER | RMSD from reference structure = 2.881 A | | | | | | | | | | |
| USER | | | | | | | | | | | |
| USER | Estimated Free Energy of Binding = −7.70 kcal/mol [= (1) + (3)] | | | | | | | | | | |
| USER | Estimated Inhibition Constant, Ki = +2.29e−06 [Temperature = 298.15 K] | | | | | | | | | | |
| USER | | | | | | | | | | | |
| USER | Final Docked Energy = −9.87 kcal/mol [= (1) + (2)] | | | | | | | | | | |
| USER | | | | | | | | | | | |
| USER | (1) Final Intermolecular Energy = −12.05 kcal/mol | | | | | | | | | | |
| USER | (2) Final Internal Energy of Ligand = +2.18 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −3.090057 16.985686 11.648671 | | | | | | | | | | |
| USER | NEWDPF quat0 −0.328188 0.860514 0.389626 −36.276032 | | | | | | | | | | |
| USER | NEWDPF ndihe 14 | | | | | | | | | | |
| USER | NEWDPF dihe0 157.96 59.07 −104.02 −174.50 9.03 58.34 −174.77 106.42 −160.68 | | | | | | | | | | |
| | 50.24 137.09 124.83 61.90 165.61 | | | | | | | | | | |
| USER | | | | | | | | | | | |
| USER | | | | Rank | x | y | z | vdW | Elec | q | RMS |
| ATOM | 1 | N1 | MOL | 8 | −0.775 | 19.649 | 6.357 | −0.38 | −0.19 | −0.211 | 2.881 |
| ATOM | 2 | C8 | MOL | 8 | 0.193 | 20.513 | 6.855 | −0.54 | +0.00 | +0.000 | 2.881 |
| ATOM | 3 | C6 | MOL | 8 | 0.268 | 21.807 | 6.508 | −0.56 | +0.00 | +0.000 | 2.881 |
| ATOM | 4 | C5 | MOL | 8 | −0.661 | 22.337 | 5.559 | −0.67 | +0.28 | +0.396 | 2.881 |
| ATOM | 5 | O11 | MOL | 8 | −0.724 | 23.495 | 5.146 | −0.04 | −0.33 | −0.396 | 2.881 |
| ATOM | 6 | N3 | MOL | 8 | −1.599 | 21.417 | 5.089 | −0.41 | −0.19 | −0.440 | 2.881 |
| ATOM | 7 | H3 | MOL | 8 | −2.292 | 21.766 | 4.413 | −0.21 | −0.08 | +0.440 | 2.881 |
| ATOM | 8 | C2 | MOL | 8 | −1.687 | 20.095 | 5.441 | −0.69 | +0.28 | +0.396 | 2.881 |
| ATOM | 9 | O10 | MOL | 8 | −2.550 | 19.353 | 4.970 | −0.26 | −0.36 | −0.396 | 2.881 |
| ATOM | 10 | C12 | MOL | 8 | −0.823 | 18.246 | 6.792 | −0.67 | +0.31 | +0.324 | 2.881 |
| ATOM | 11 | O22 | MOL | 8 | −1.110 | 18.269 | 8.185 | −0.07 | −0.23 | −0.227 | 2.881 |
| ATOM | 12 | C20 | MOL | 8 | −0.666 | 17.022 | 8.786 | −0.65 | +0.10 | +0.113 | 2.881 |
| ATOM | 13 | C25 | MOL | 8 | 0.031 | 17.340 | 10.110 | −0.57 | +0.10 | +0.113 | 2.881 |
| ATOM | 14 | O28 | MOL | 8 | 1.283 | 17.926 | 9.811 | −0.02 | −0.38 | −0.368 | 2.881 |
| ATOM | 15 | P29 | MOL | 8 | 2.713 | 17.820 | 10.468 | −0.52 | +1.35 | +1.019 | 2.881 |
| ATOM | 16 | O30 | MOL | 8 | 2.640 | 16.800 | 11.552 | −0.10 | −0.29 | −0.255 | 2.881 |
| ATOM | 17 | O31 | MOL | 8 | 3.725 | 17.630 | 9.381 | −0.13 | −0.48 | −0.255 | 2.881 |
| ATOM | 18 | O32 | MOL | 8 | 2.701 | 19.283 | 11.103 | +0.00 | −0.65 | −0.510 | 2.881 |
| ATOM | 19 | P33 | MOL | 8 | 3.167 | 19.865 | 12.519 | −0.47 | +1.17 | +1.019 | 2.881 |
| ATOM | 20 | O35 | MOL | 8 | 4.566 | 20.260 | 12.213 | −0.37 | −0.36 | −0.255 | 2.881 |
| ATOM | 21 | O36 | MOL | 8 | 2.872 | 18.982 | 13.675 | −0.10 | −0.26 | −0.255 | 2.881 |
| ATOM | 22 | O34 | MOL | 8 | 2.225 | 21.160 | 12.759 | +0.01 | −0.39 | −0.368 | 2.881 |
| ATOM | 23 | C37 | MOL | 8 | 2.344 | 21.895 | 15.354 | −0.31 | +0.20 | +0.227 | 2.881 |
| ATOM | 24 | O39 | MOL | 8 | 1.152 | 22.268 | 15.245 | −0.07 | −0.19 | −0.227 | 2.881 |
| ATOM | 25 | C40 | MOL | 8 | 0.036 | 21.361 | 15.103 | −0.69 | +0.08 | +0.113 | 2.881 |
| ATOM | 26 | C50 | MOL | 8 | −1.181 | 22.228 | 15.366 | −0.76 | +0.10 | +0.113 | 2.881 |
| ATOM | 27 | O61 | MOL | 8 | −0.922 | 23.557 | 15.078 | −0.29 | −0.71 | −0.537 | 2.881 |
| ATOM | 28 | H61 | MOL | 8 | 0.021 | 23.711 | 15.243 | +0.04 | +0.52 | +0.424 | 2.881 |
| ATOM | 29 | C41 | MOL | 8 | 0.228 | 20.225 | 16.092 | +0.35 | +0.02 | +0.113 | 2.881 |
| ATOM | 30 | O48 | MOL | 8 | −0.752 | 19.231 | 15.919 | −0.10 | −0.33 | −0.537 | 2.881 |
| ATOM | 31 | H48 | MOL | 8 | −1.610 | 19.606 | 16.055 | +0.11 | +0.20 | +0.424 | 2.881 |
| ATOM | 32 | C42 | MOL | 8 | 1.576 | 19.566 | 15.851 | −0.33 | +0.06 | +0.113 | 2.881 |
| ATOM | 33 | O46 | MOL | 8 | 1.802 | 18.528 | 16.756 | +0.08 | −0.23 | −0.537 | 2.881 |
| ATOM | 34 | H46 | MOL | 8 | 0.972 | 18.328 | 17.171 | −0.06 | +0.06 | +0.424 | 2.881 |
| ATOM | 35 | C43 | MOL | 8 | 2.692 | 20.593 | 16.033 | −0.34 | +0.13 | +0.211 | 2.881 |
| ATOM | 36 | N51 | MOL | 8 | 3.971 | 20.094 | 15.620 | −0.15 | −0.51 | −0.650 | 2.881 |
| ATOM | 37 | H51 | MOL | 8 | 4.142 | 19.955 | 14.614 | +0.08 | +0.37 | +0.440 | 2.881 |
| ATOM | 38 | C53 | MOL | 8 | 4.966 | 19.796 | 16.493 | −0.29 | +0.34 | +0.396 | 2.881 |
| ATOM | 39 | O54 | MOL | 8 | 4.921 | 20.061 | 17.662 | +0.10 | −0.35 | −0.396 | 2.881 |
| ATOM | 40 | C55 | MOL | 8 | 6.140 | 19.069 | 15.877 | −0.38 | +0.00 | +0.000 | 2.881 |
| ATOM | 41 | C18 | MOL | 8 | 0.268 | 16.384 | 7.757 | −0.69 | +0.07 | +0.113 | 2.881 |
| ATOM | 42 | O23 | MOL | 8 | −0.424 | 15.344 | 7.060 | +0.04 | −0.18 | −0.537 | 2.881 |
| ATOM | 43 | H23 | MOL | 8 | −1.237 | 15.743 | 6.568 | +0.03 | +0.30 | +0.424 | 2.881 |
| ATOM | 44 | C14 | MOL | 8 | 0.523 | 17.519 | 6.739 | −0.65 | +0.09 | +0.113 | 2.881 |
| ATOM | 45 | O16 | MOL | 8 | 0.739 | 16.974 | 5.458 | +0.01 | −0.43 | −0.537 | 2.881 |
| ATOM | 46 | H16 | MOL | 8 | 1.693 | 17.203 | 5.144 | +0.05 | +0.37 | +0.424 | 2.881 |
| TER | | | | | | | | | | | |
| ENDMDL | | | | | | | | | | | |
| MODEL | 90 | | | | | | | | | | |
| USER | Run = 90 | | | | | | | | | | |
| USER | Cluster Rank = 9 | | | | | | | | | | |
| USER | Number of conformations in this cluster = 1 | | | | | | | | | | |
| USER | | | | | | | | | | | |
| USER | RMSD from reference structure = 1.901 A | | | | | | | | | | |
| USER | | | | | | | | | | | |
| USER | Estimated Free Energy of Binding = −9.53 kcal/mol [= (1) + (3)] | | | | | | | | | | |
| USER | Estimated Inhibition Constant, Ki = +1.04e−07 [Temperature = 298.15 K] | | | | | | | | | | |
| USER | | | | | | | | | | | |
| USER | Final Docked Energy = −13.83 kcal/mol [= (1) + (2)] | | | | | | | | | | |
| USER | | | | | | | | | | | |
| USER | (1) Final Intermolecular Energy = −13.88 kcal/mol | | | | | | | | | | |
| USER | (2) Final Internal Energy of Ligand = +0.05 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 1.490671 19.819369 12.866707 | | | | | | | | | | |
| USER | NEWDPF quat0 0.663104 −0.361566 0.655410 −29.208596 | | | | | | | | | | |
| USER | NEWDPF ndihe 14 | | | | | | | | | | |
| USER | NEWDPF dihe0 93.86 −175.64 −14.28 −163.19 170.56 163.57 −174.24 −57.45 | | | | | | | | | | |
| | 22.38 36.00 58.92 76.86 53.61 −7.32 | | | | | | | | | | |
| USER | | | | | | | | | | | |
| USER | | | | Rank | x | y | z | vdW | Elec | q | RMS |
| ATOM | 1 | N1 | MOL | 9 | −0.755 | 19.943 | 6.917 | −0.38 | −0.19 | −0.211 | 1.901 |
| ATOM | 2 | C8 | MOL | 9 | 0.258 | 20.599 | 6.228 | −0.55 | +0.00 | +0.000 | 1.901 |
| ATOM | 3 | C6 | MOL | 9 | 0.035 | 21.644 | 5.417 | −0.54 | +0.00 | +0.000 | 1.901 |
| ATOM | 4 | C5 | MOL | 9 | −1.303 | 22.103 | 5.211 | −0.68 | +0.22 | +0.396 | 1.901 |
| ATOM | 5 | O11 | MOL | 9 | −1.667 | 23.045 | 4.508 | −0.10 | −0.24 | −0.396 | 1.901 |
| ATOM | 6 | N3 | MOL | 9 | −2.274 | 21.398 | 5.922 | −0.51 | −0.21 | −0.440 | 1.901 |
| ATOM | 7 | H3 | MOL | 9 | −3.250 | 21.708 | 5.809 | −0.26 | +0.00 | +0.440 | 1.901 |
| ATOM | 8 | C2 | MOL | 9 | −2.056 | 20.332 | 6.757 | −0.80 | +0.37 | +0.396 | 1.901 |
| ATOM | 9 | O10 | MOL | 9 | −2.979 | 19.765 | 7.343 | −0.41 | −0.54 | −0.396 | 1.901 |
| ATOM | 10 | C12 | MOL | 9 | −0.448 | 18.818 | 7.811 | −0.57 | +0.31 | +0.324 | 1.901 |
| ATOM | 11 | O22 | MOL | 9 | 0.353 | 19.348 | 8.861 | +0.05 | −0.23 | −0.227 | 1.901 |
| ATOM | 12 | C20 | MOL | 9 | 1.119 | 18.273 | 9.469 | −0.52 | +0.11 | +0.113 | 1.901 |
| ATOM | 13 | C25 | MOL | 9 | 2.548 | 18.764 | 9.704 | −0.52 | +0.14 | +0.113 | 1.901 |
| ATOM | 14 | O28 | MOL | 9 | 2.768 | 18.808 | 11.101 | +0.01 | −0.47 | −0.368 | 1.901 |
| ATOM | 15 | P29 | MOL | 9 | 3.685 | 19.697 | 12.025 | −0.51 | +1.33 | +1.019 | 1.901 |
| ATOM | 16 | O30 | MOL | 9 | 5.016 | 19.816 | 11.365 | −0.41 | −0.44 | −0.255 | 1.901 |
| ATOM | 17 | O31 | MOL | 9 | 2.946 | 20.954 | 12.364 | −0.11 | −0.29 | −0.255 | 1.901 |
| ATOM | 18 | O32 | MOL | 9 | 3.769 | 18.644 | 13.221 | +0.10 | −0.59 | −0.510 | 1.901 |
| ATOM | 19 | P33 | MOL | 9 | 4.311 | 17.142 | 13.319 | −0.23 | +1.53 | +1.019 | 1.901 |
| ATOM | 20 | O35 | MOL | 9 | 5.776 | 17.377 | 13.379 | −0.22 | −0.40 | −0.255 | 1.901 |
| ATOM | 21 | O36 | MOL | 9 | 3.740 | 16.214 | 12.311 | −0.37 | −0.46 | −0.255 | 1.901 |
| ATOM | 22 | O34 | MOL | 9 | 3.740 | 16.593 | 14.732 | +0.06 | −0.34 | −0.368 | 1.901 |
| ATOM | 23 | C37 | MOL | 9 | 2.782 | 18.658 | 16.183 | −0.27 | +0.16 | +0.227 | 1.901 |
| ATOM | 24 | O39 | MOL | 9 | 2.159 | 19.018 | 15.157 | +0.09 | −0.19 | −0.227 | 1.901 |
| ATOM | 25 | C40 | MOL | 9 | 1.024 | 18.305 | 14.615 | −0.33 | +0.14 | +0.113 | 1.901 |
| ATOM | 26 | C50 | MOL | 9 | 0.409 | 19.282 | 13.630 | −0.50 | +0.15 | +0.113 | 1.901 |
| ATOM | 27 | O61 | MOL | 9 | 0.174 | 18.678 | 12.407 | −0.05 | −0.83 | −0.537 | 1.901 |
| ATOM | 28 | H61 | MOL | 9 | 0.942 | 18.856 | 11.842 | +0.06 | +0.52 | +0.424 | 1.901 |
| ATOM | 29 | C41 | MOL | 9 | 0.118 | 17.922 | 15.773 | −0.58 | +0.10 | +0.113 | 1.901 |
| ATOM | 30 | O48 | MOL | 9 | −0.944 | 17.108 | 15.337 | +0.05 | −0.73 | −0.537 | 1.901 |
| ATOM | 31 | H48 | MOL | 9 | −0.602 | 16.284 | 15.022 | −0.20 | +0.58 | +0.424 | 1.901 |
| ATOM | 32 | C42 | MOL | 9 | 0.908 | 17.114 | 16.788 | −0.54 | +0.08 | +0.113 | 1.901 |
| ATOM | 33 | O46 | MOL | 9 | 0.116 | 16.760 | 17.881 | −0.17 | −0.41 | −0.537 | 1.901 |
| ATOM | 34 | H46 | MOL | 9 | −0.107 | 15.843 | 17.785 | +0.05 | +0.49 | +0.424 | 1.901 |
| ATOM | 35 | C43 | MOL | 9 | 2.077 | 17.948 | 17.313 | −0.33 | +0.11 | +0.211 | 1.901 |
| ATOM | 36 | N51 | MOL | 9 | 2.982 | 17.180 | 18.116 | −0.27 | −0.53 | −0.650 | 1.901 |
| ATOM | 37 | H51 | MOL | 9 | 3.581 | 16.483 | 17.651 | +0.08 | +0.38 | +0.440 | 1.901 |
| ATOM | 38 | C53 | MOL | 9 | 3.086 | 17.329 | 19.460 | −0.54 | +0.44 | +0.396 | 1.901 |
| ATOM | 39 | O54 | MOL | 9 | 2.547 | 18.208 | 20.072 | −0.46 | −0.59 | −0.396 | 1.901 |
| ATOM | 40 | C55 | MOL | 9 | 3.922 | 16.270 | 20.144 | −0.60 | +0.00 | +0.000 | 1.901 |
| ATOM | 41 | C18 | MOL | 9 | 1.042 | 17.108 | 8.481 | −0.59 | +0.09 | +0.113 | 1.901 |
| ATOM | 42 | O23 | MOL | 9 | 0.081 | 16.153 | 8.939 | −0.20 | −0.39 | −0.537 | 1.901 |
| ATOM | 43 | H23 | MOL | 9 | −0.869 | 16.537 | 8.819 | +0.06 | +0.37 | +0.424 | 1.901 |
| ATOM | 44 | C14 | MOL | 9 | 0.465 | 17.752 | 7.199 | −0.60 | +0.09 | +0.113 | 1.901 |
| ATOM | 45 | O16 | MOL | 9 | −0.294 | 16.804 | 6.486 | −0.23 | −0.41 | −0.537 | 1.901 |
| ATOM | 46 | H16 | MOL | 9 | −0.896 | 16.283 | 7.141 | +0.04 | +0.34 | +0.424 | 1.901 |
| TER | | | | | | | | | | | |
| ENDMDL | | | | | | | | | | | |
| MODEL | 66 | | | | | | | | | | |
| USER | Run = 66 | | | | | | | | | | |
| USER | Cluster Rank = 10 | | | | | | | | | | |
| USER | Number of conformations in this cluster = 1 | | | | | | | | | | |
| USER | | | | | | | | | | | |
| USER | RMSD from reference structure = 2.182 A | | | | | | | | | | |
| USER | | | | | | | | | | | |
| USER | Estimated Free Energy of Binding = −9.79 kcal/mol [= (1) + (3)] | | | | | | | | | | |
| USER | Estimated Inhibition Constant, Ki = +6.72e−08 [Temperature = 298.15 K] | | | | | | | | | | |
| USER | | | | | | | | | | | |
| USER | Final Docked Energy = −13.67 kcal/mol [= (1) + (2)] | | | | | | | | | | |
| USER | | | | | | | | | | | |
| USER | (1) Final Intermolecular Energy = −14.14 kcal/mol | | | | | | | | | | |
| USER | (2) Final Internal Energy of Ligand = +0.48 kcal/mol | | | | | | | | | | |
| USER | (3) Torsional Free Energy = +4.36 kcal/mol | | | | | | | | | | |
| USER | | | | | | | | | | | |
| USER | | | | | | | | | | | |
| USER | DPF = gglc.dpf | | | | | | | | | | |
| USER | NEWDPF move gglc.pdbq | | | | | | | | | | |
| USER | NEWDPF about 1.374000 18.170000 13.289000 | | | | | | | | | | |
| USER | NEWDPF tran0 −0.939938 19.145584 13.627042 | | | | | | | | | | |
| USER | NEWDPF quat0 0.566730 0.397973 0.721412 −23.305526 | | | | | | | | | | |
| USER | NEWDPF ndihe 14 | | | | | | | | | | |
| USER | NEWDPF dihe0 22.47 179.97 52.78 −60.27 −179.98 175.37 −158.71 −49.97 −114.52 | | | | | | | | | | |
| | −132.20 109.02 30.33 92.09 −2.77 | | | | | | | | | | |
| USER | | | | | | | | | | | |
| USER | | | | Rank | x | y | z | vdW | Elec | q | RMS |
| ATOM | 1 | N1 | MOL | 10 | −1.050 | 19.493 | 7.277 | −0.39 | −0.21 | −0.211 | 2.182 |
| ATOM | 2 | C8 | MOL | 10 | 0.068 | 20.271 | 7.000 | −0.53 | +0.00 | +0.000 | 2.182 |
| ATOM | 3 | C6 | MOL | 10 | 0.031 | 21.348 | 6.201 | −0.60 | +0.00 | +0.000 | 2.182 |
| ATOM | 4 | C5 | MOL | 10 | −1.198 | 21.713 | 5.567 | −0.71 | +0.24 | +0.396 | 2.182 |
| ATOM | 5 | O11 | MOL | 10 | −1.393 | 22.668 | 4.816 | −0.15 | −0.23 | −0.396 | 2.182 |
| ATOM | 6 | N3 | MOL | 10 | −2.278 | 20.887 | 5.877 | −0.52 | −0.24 | −0.440 | 2.182 |
| ATOM | 7 | H3 | MOL | 10 | −3.184 | 21.126 | 5.448 | −0.26 | −0.11 | +0.440 | 2.182 |
| ATOM | 8 | C2 | MOL | 10 | −2.252 | 19.788 | 6.696 | −0.84 | +0.43 | +0.396 | 2.182 |
| ATOM | 9 | O10 | MOL | 10 | −3.259 | 19.111 | 6.910 | −0.24 | −0.85 | −0.396 | 2.182 |
| ATOM | 10 | C12 | MOL | 10 | −0.954 | 18.338 | 8.180 | −0.63 | +0.33 | +0.324 | 2.182 |
| ATOM | 11 | O22 | MOL | 10 | −0.609 | 18.858 | 9.459 | −0.03 | −0.23 | −0.227 | 2.182 |
| ATOM | 12 | C20 | MOL | 10 | 0.007 | 17.807 | 10.253 | −0.57 | +0.11 | +0.113 | 2.182 |
| ATOM | 13 | C25 | MOL | 10 | 1.219 | 18.393 | 10.980 | −0.47 | +0.13 | +0.113 | 2.182 |
| ATOM | 14 | O28 | MOL | 10 | 0.994 | 18.263 | 12.371 | +0.01 | −0.54 | −0.368 | 2.182 |
| ATOM | 15 | P29 | MOL | 10 | 0.740 | 19.303 | 13.528 | −0.45 | +1.30 | +1.019 | 2.182 |
| ATOM | 16 | O30 | MOL | 10 | 2.065 | 19.692 | 14.089 | +0.07 | −0.25 | −0.255 | 2.182 |
| ATOM | 17 | O31 | MOL | 10 | −0.157 | 20.384 | 13.010 | +0.10 | −0.33 | −0.255 | 2.182 |
| ATOM | 18 | O32 | MOL | 10 | 0.022 | 18.270 | 14.510 | −0.30 | −0.85 | −0.510 | 2.182 |
| ATOM | 19 | P33 | MOL | 10 | 0.452 | 17.600 | 15.897 | −0.55 | +0.92 | +1.019 | 2.182 |
| ATOM | 20 | O35 | MOL | 10 | 1.125 | 16.367 | 15.416 | +0.02 | −0.24 | −0.255 | 2.182 |
| ATOM | 21 | O36 | MOL | 10 | −0.635 | 17.517 | 16.904 | −0.12 | −0.17 | −0.255 | 2.182 |
| ATOM | 22 | O34 | MOL | 10 | 1.512 | 18.641 | 16.543 | +0.06 | −0.15 | −0.368 | 2.182 |
| ATOM | 23 | C37 | MOL | 10 | 3.303 | 17.289 | 18.045 | −0.41 | +0.19 | +0.227 | 2.182 |
| ATOM | 24 | O39 | MOL | 10 | 3.561 | 16.608 | 17.025 | +0.05 | −0.19 | −0.227 | 2.182 |
| ATOM | 25 | C40 | MOL | 10 | 4.264 | 17.124 | 15.872 | −0.33 | +0.10 | +0.113 | 2.182 |
| ATOM | 26 | C50 | MOL | 10 | 4.644 | 15.884 | 15.085 | −0.42 | +0.11 | +0.113 | 2.182 |
| ATOM | 27 | O61 | MOL | 10 | 4.544 | 16.105 | 13.722 | −0.29 | −0.71 | −0.537 | 2.182 |
| ATOM | 28 | H61 | MOL | 10 | 4.571 | 15.240 | 13.284 | +0.05 | +0.52 | +0.424 | 2.182 |
| ATOM | 29 | C41 | MOL | 10 | 5.443 | 17.942 | 16.371 | −0.32 | +0.11 | +0.113 | 2.182 |
| ATOM | 30 | O48 | MOL | 10 | 6.098 | 18.588 | 15.307 | +0.08 | −0.52 | −0.537 | 2.182 |
| ATOM | 31 | H48 | MOL | 10 | 5.496 | 19.176 | 14.875 | +0.10 | +0.39 | +0.424 | 2.182 |
| ATOM | 32 | C42 | MOL | 10 | 4.945 | 19.028 | 17.309 | −0.32 | +0.10 | +0.113 | 2.182 |
| ATOM | 33 | O46 | MOL | 10 | 6.001 | 19.790 | 17.809 | +0.07 | −0.56 | −0.537 | 2.182 |
| ATOM | 34 | H46 | MOL | 10 | 5.625 | 20.529 | 18.270 | +0.09 | +0.42 | +0.424 | 2.182 |
| ATOM | 35 | C43 | MOL | 10 | 4.227 | 18.392 | 18.500 | −0.39 | +0.20 | +0.211 | 2.182 |
| ATOM | 36 | N51 | MOL | 10 | 3.554 | 19.359 | 19.315 | −0.26 | −0.62 | −0.650 | 2.182 |
| ATOM | 37 | H51 | MOL | 10 | 2.594 | 19.631 | 19.063 | −0.17 | +0.17 | +0.440 | 2.182 |
| ATOM | 38 | C53 | MOL | 10 | 4.124 | 19.931 | 20.406 | −0.52 | +0.56 | +0.396 | 2.182 |
| ATOM | 39 | O54 | MOL | 10 | 5.172 | 19.575 | 20.868 | −0.28 | −0.72 | −0.396 | 2.182 |
| ATOM | 40 | C55 | MOL | 10 | 3.340 | 21.087 | 20.986 | −0.52 | +0.00 | +0.000 | 2.182 |
| ATOM | 41 | C18 | MOL | 10 | 0.381 | 16.709 | 9.256 | −0.67 | +0.09 | +0.113 | 2.182 |
| ATOM | 42 | O23 | MOL | 10 | −0.580 | 15.652 | 9.322 | −0.25 | −0.43 | −0.537 | 2.182 |
| ATOM | 43 | H23 | MOL | 10 | −0.374 | 14.955 | 8.591 | −0.08 | +0.26 | +0.424 | 2.182 |
| ATOM | 44 | C14 | MOL | 10 | 0.210 | 17.390 | 7.878 | −0.62 | +0.09 | +0.113 | 2.182 |
| ATOM | 45 | O16 | MOL | 10 | −0.168 | 16.435 | 6.915 | −0.24 | −0.35 | −0.537 | 2.182 |
| ATOM | 46 | H16 | MOL | 10 | −1.005 | 15.930 | 7.242 | +0.03 | +0.32 | +0.424 | 2.182 |
| TER | | | | | | | | | | | |
| ENDMDL | | | | | | | | | | | |

TABLE 18
UDPGlcNAc
CLUSTERING HISTOGRAM

| | Lowest | | Mean | Num | | | | | | | |
|---|---|---|---|---|---|---|---|---|---|---|---|
| Cluster | Docked | | Docked | in | Histogram | | | | | | |
| Rank | Energy | Run | Energy | Clus | 5 | 10 | 15 | 20 | 25 | 30 | 35 |
| 1 | −11.34 | 30 | −11.34 | 1 | # | | | | | | |
| 2 | −11.18 | 31 | −11.18 | 1 | # | | | | | | |
| 3 | −11.15 | 5 | −11.15 | 1 | # | | | | | | |
| 4 | −11.01 | 33 | −11.01 | 1 | # | | | | | | |
| 5 | −10.99 | 38 | −10.79 | 2 | ## | | | | | | |
| 6 | −10.52 | 3 | −9.86 | 2 | ## | | | | | | |
| 7 | −10.39 | 48 | −10.39 | 1 | # | | | | | | |
| 8 | −10.39 | 12 | −10.39 | 1 | # | | | | | | |
| 9 | −9.79 | 39 | −9.79 | 1 | # | | | | | | |
| 10 | −9.65 | 28 | −9.65 | 1 | # | | | | | | |
| 11 | −9.40 | 44 | −9.40 | 1 | # | | | | | | |
| 12 | −9.27 | 36 | −9.27 | 1 | # | | | | | | |
| 13 | −9.21 | 11 | −9.21 | 1 | # | | | | | | |
| 14 | −9.20 | 27 | −9.20 | 1 | # | | | | | | |
| 15 | −9.07 | 9 | −9.07 | 1 | # | | | | | | |
| 16 | −9.00 | 45 | −9.00 | 1 | # | | | | | | |
| 17 | −8.64 | 15 | −8.64 | 1 | # | | | | | | |
| 18 | −8.60 | 14 | −8.60 | 1 | # | | | | | | |
| 19 | −8.58 | 1 | −8.58 | 1 | # | | | | | | |
| 20 | −8.47 | 23 | −8.47 | 1 | # | | | | | | |
| 21 | −8.37 | 32 | −8.37 | 1 | # | | | | | | |
| 22 | −8.27 | 8 | −8.27 | 1 | # | | | | | | |
| 23 | −8.20 | 41 | −8.20 | 1 | # | | | | | | |
| 24 | −8.17 | 21 | −8.17 | 1 | # | | | | | | |
| 25 | −8.08 | 42 | −7.14 | 2 | ## | | | | | | |
| 26 | −8.06 | 4 | −8.06 | 1 | # | | | | | | |
| 27 | −8.05 | 29 | −8.05 | 1 | # | | | | | | |
| 28 | −7.94 | 13 | −7.94 | 1 | # | | | | | | |
| 29 | −7.90 | 16 | −7.90 | 1 | # | | | | | | |
| 30 | −7.81 | 22 | −7.81 | 1 | # | | | | | | |
| 31 | −7.81 | 17 | −7.81 | 1 | # | | | | | | |
| 32 | −7.75 | 6 | −7.75 | 1 | # | | | | | | |
| 33 | −7.72 | 49 | −7.72 | 1 | # | | | | | | |
| 34 | −7.72 | 26 | −7.72 | 1 | # | | | | | | |
| 35 | −7.71 | 47 | −7.71 | 1 | # | | | | | | |
| 36 | −7.38 | 24 | −7.38 | 1 | # | | | | | | |
| 37 | −7.32 | 34 | −7.32 | 1 | # | | | | | | |
| 38 | −7.09 | 43 | −7.09 | 1 | # | | | | | | |
| 39 | −6.96 | 25 | −6.96 | 1 | # | | | | | | |
| 40 | −6.68 | 46 | −6.68 | 1 | # | | | | | | |
| 41 | −6.63 | 19 | −6.63 | 1 | # | | | | | | |
| 42 | −6.58 | 50 | −6.58 | 1 | # | | | | | | |
| 43 | −6.56 | 7 | −6.56 | 1 | # | | | | | | |
| 44 | −6.55 | 2 | −6.55 | 1 | # | | | | | | |
| 45 | −6.48 | 35 | −6.48 | 1 | # | | | | | | |
| 46 | −5.82 | 18 | −5.82 | 1 | # | | | | | | |
| 47 | −5.45 | 10 | −5.45 | 1 | # | | | | | | |

Number of multi-member conformational clusters found = 3, out of 50 runs.

RMSD TABLE

| | Sub- | | Docked | Cluster | Reference | Grep |
|---|---|---|---|---|---|---|
| Rank | Rank | Run | Energy | RMSD | RMSD | Pattern |
| 1 | 1 | 30 | −11.34 | 0.00 | 2.63 | RANKING |
| 2 | 1 | 31 | −11.18 | 0.00 | 2.69 | RANKING |
| 3 | 1 | 5 | −11.15 | 0.00 | 2.45 | RANKING |
| 4 | 1 | 33 | −11.01 | 0.00 | 3.26 | RANKING |
| 5 | 1 | 38 | −10.99 | 0.00 | 2.24 | RANKING |
| 5 | 2 | 37 | −10.58 | 1.12 | 2.43 | RANKING |
| 6 | 1 | 3 | −10.52 | 0.00 | 3.26 | RANKING |
| 6 | 2 | 20 | −9.20 | 1.45 | 3.72 | RANKING |
| 7 | 1 | 48 | −10.39 | 0.00 | 2.80 | RANKING |
| 8 | 1 | 12 | −10.39 | 0.00 | 2.44 | RANKING |
| 9 | 1 | 39 | −9.79 | 0.00 | 5.53 | RANKING |
| 10 | 1 | 28 | −9.65 | 0.00 | 4.36 | RANKING |
| 11 | 1 | 44 | −9.40 | 0.00 | 3.88 | RANKING |
| 12 | 1 | 36 | −9.27 | 0.00 | 3.26 | RANKING |
| 13 | 1 | 11 | −9.21 | 0.00 | 6.22 | RANKING |
| 14 | 1 | 27 | −9.20 | 0.00 | 2.64 | RANKING |
| 15 | 1 | 9 | −9.07 | 0.00 | 3.50 | RANKING |
| 16 | 1 | 45 | −9.00 | 0.00 | 4.11 | RANKING |
| 17 | 1 | 15 | −8.64 | 0.00 | 2.48 | RANKING |
| 18 | 1 | 14 | −8.60 | 0.00 | 2.56 | RANKING |
| 19 | 1 | 1 | −8.58 | 0.00 | 4.13 | RANKING |
| 20 | 1 | 23 | −8.47 | 0.00 | 6.00 | RANKING |
| 21 | 1 | 32 | −8.37 | 0.00 | 3.57 | RANKING |
| 22 | 1 | 8 | −8.27 | 0.00 | 2.84 | RANKING |
| 23 | 1 | 41 | −8.20 | 0.00 | 4.25 | RANKING |
| 24 | 1 | 21 | −8.17 | 0.00 | 2.76 | RANKING |
| 25 | 1 | 42 | −8.08 | 0.00 | 4.06 | RANKING |
| 25 | 2 | 40 | −6.19 | 1.49 | 3.93 | RANKING |
| 26 | 1 | 4 | −8.06 | 0.00 | 3.50 | RANKING |
| 27 | 1 | 29 | −8.05 | 0.00 | 3.82 | RANKING |
| 28 | 1 | 13 | −7.94 | 0.00 | 3.89 | RANKING |
| 29 | 1 | 16 | −7.90 | 0.00 | 2.61 | RANKING |
| 30 | 1 | 22 | −7.81 | 0.00 | 4.98 | RANKING |
| 31 | 1 | 17 | −7.81 | 0.00 | 3.10 | RANKING |
| 32 | 1 | 6 | −7.75 | 0.00 | 3.04 | RANKING |
| 33 | 1 | 49 | −7.72 | 0.00 | 4.28 | RANKING |
| 34 | 1 | 26 | −7.72 | 0.00 | 4.46 | RANKING |
| 35 | 1 | 47 | −7.71 | 0.00 | 3.63 | RANKING |
| 36 | 1 | 24 | −7.38 | 0.00 | 2.64 | RANKING |
| 37 | 1 | 34 | −7.32 | 0.00 | 3.41 | RANKING |
| 38 | 1 | 43 | −7.09 | 0.00 | 6.14 | RANKING |
| 39 | 1 | 25 | −6.96 | 0.00 | 4.18 | RANKING |
| 40 | 1 | 46 | −6.68 | 0.00 | 3.65 | RANKING |
| 41 | 1 | 19 | −6.63 | 0.00 | 4.04 | RANKING |
| 42 | 1 | 50 | −6.58 | 0.00 | 5.44 | RANKING |
| 43 | 1 | 7 | −6.56 | 0.00 | 4.46 | RANKING |
| 44 | 1 | 2 | −6.55 | 0.00 | 3.45 | RANKING |
| 45 | 1 | 35 | −6.48 | 0.00 | 3.84 | RANKING |
| 46 | 1 | 18 | −5.82 | 0.00 | 4.04 | RANKING |
| 47 | 1 | 10 | −5.45 | 0.00 | 7.17 | RANKING |

LOWEST ENERGY DOCKED CONFORMATION from EACH CLUSTER
Keeping original residue number (specified in the input PDBQ file) for outputing.

| MODEL | 30 | | | | | | | | | | |
|---|---|---|---|---|---|---|---|---|---|---|---|
| USER | Run = 30 | | | | | | | | | | |
| USER | Cluster Rank = 1 | | | | | | | | | | |
| USER | Number of conformations in this cluster = 1 | | | | | | | | | | |
| USER | | | | | | | | | | | |
| USER | RMSD from reference structure = 2.634 A | | | | | | | | | | |
| USER | | | | | | | | | | | |
| USER | Estimated Free Energy of Binding = −8.89 kcal/mol [= (1) + (3)] | | | | | | | | | | |
| USER | Estimated Inhibition Constant, Ki = +3.05e−07 [Temperature = 298.15 K] | | | | | | | | | | |
| USER | | | | | | | | | | | |
| USER | Final Docked Energy = −11.34 kcal/mol [= (1) + (2)] | | | | | | | | | | |
| USER | | | | | | | | | | | |
| USER | (1) Final Intermolecular Energy = −13.56 kcal/mol | | | | | | | | | | |
| USER | (2) Final Internal Energy of Ligand = +2.21 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −1.771499 −3.854861 −3.264925 | | | | | | | | | | |
| USER | NEWDPF quat0 −0.157682 −0.879023 −0.449950 −31.528235 | | | | | | | | | | |
| USER | NEWDPF ndihe 15 | | | | | | | | | | |
| USER | NEWDPF dihe0 −176.12 100.88 −121.76 −2.75 64.17 156.47 0.22 −6.95 −34.89 | | | | | | | | | | |
| | −38.42 119.42 −28.27 −86.37 −86.48 62.15 | | | | | | | | | | |
| USER | | | | | | | | | | | |
| USER | | | | | x | y | z | vdW | Elec | q | RMS |
| ATOM | 1 | C2 | URI | 1 | −1.246 | −11.758 | −6.106 | −0.66 | +0.25 | +0.296 | 2.634 |
| ATOM | 2 | N3 | URI | 1 | −1.176 | −12.992 | −5.509 | −0.38 | −0.20 | −0.292 | 2.634 |
| ATOM | 3 | C4 | URI | 1 | −1.633 | −13.319 | −4.231 | −0.65 | +0.25 | +0.346 | 2.634 |
| ATOM | 4 | C5 | URI | 1 | −2.210 | −12.228 | −3.510 | −0.45 | +0.00 | +0.000 | 2.634 |
| ATOM | 5 | C6 | URI | 1 | −2.287 | −11.019 | −4.086 | −0.39 | −0.04 | −0.050 | 2.634 |
| ATOM | 6 | N1 | URI | 1 | −1.802 | −10.755 | −5.363 | −0.31 | +0.03 | +0.039 | 2.634 |
| ATOM | 7 | O8 | URI | 1 | −1.487 | −14.477 | −3.841 | −0.23 | −0.33 | −0.396 | 2.634 |
| ATOM | 8 | O7 | URI | 1 | −0.819 | −11.564 | −7.245 | −0.26 | −0.39 | −0.396 | 2.634 |
| ATOM | 9 | H3 | URI | 1 | −0.754 | −13.730 | −6.044 | −0.14 | +0.32 | +0.393 | 2.634 |
| ATOM | 10 | C9 | URI | 1 | −1.895 | −9.407 | −5.935 | −0.45 | +0.11 | +0.174 | 2.634 |
| ATOM | 11 | O14 | URI | 1 | −0.570 | −9.029 | −6.286 | −0.01 | −0.12 | −0.227 | 2.634 |
| ATOM | 12 | C13 | URI | 1 | −0.487 | −7.580 | −6.357 | −0.44 | +0.03 | +0.113 | 2.634 |
| ATOM | 13 | C16 | URI | 1 | 0.810 | −7.133 | −5.683 | −0.41 | +0.04 | +0.113 | 2.634 |
| ATOM | 14 | O17 | URI | 1 | 1.815 | −7.066 | −6.678 | −0.02 | −0.17 | −0.368 | 2.634 |
| ATOM | 15 | P18 | URI | 1 | 2.804 | −5.929 | −7.144 | −0.50 | +0.85 | +1.210 | 2.634 |
| ATOM | 16 | O19 | URI | 1 | 3.054 | −6.112 | −8.602 | +0.02 | −0.70 | −0.850 | 2.634 |
| ATOM | 17 | O20 | URI | 1 | 2.266 | −4.611 | −6.676 | +0.04 | −0.55 | −0.850 | 2.634 |
| ATOM | 18 | O21 | URI | 1 | 4.056 | −6.459 | −6.310 | −0.09 | −0.50 | −0.510 | 2.634 |
| ATOM | 19 | P22 | URI | 1 | 4.555 | −6.213 | −4.811 | −0.55 | +1.42 | +1.210 | 2.634 |
| ATOM | 20 | O25 | URI | 1 | 5.025 | −7.434 | −4.110 | −0.26 | −1.13 | −0.850 | 2.634 |
| ATOM | 21 | O24 | URI | 1 | 5.473 | −5.062 | −5.006 | +0.07 | −1.54 | −0.850 | 2.634 |
| ATOM | 22 | O23 | URI | 1 | 3.226 | −5.781 | −4.002 | +0.01 | −0.30 | −0.368 | 2.634 |
| ATOM | 23 | C37 | URI | 1 | 3.750 | −4.876 | −3.057 | −0.42 | +0.23 | +0.227 | 2.634 |
| ATOM | 24 | C45 | URI | 1 | 2.695 | −4.537 | −1.958 | −0.30 | +0.14 | +0.211 | 2.634 |
| ATOM | 25 | N41 | URI | 1 | 2.184 | −5.800 | −1.357 | −0.21 | −0.31 | −0.650 | 2.634 |
| ATOM | 26 | H41 | URI | 1 | 2.259 | −6.623 | −1.956 | +0.08 | +0.20 | +0.440 | 2.634 |
| ATOM | 27 | C49 | URI | 1 | 1.742 | −5.892 | −0.099 | −0.28 | +0.13 | +0.396 | 2.634 |
| ATOM | 28 | O38 | URI | 1 | 1.652 | −4.935 | 0.671 | +0.17 | −0.10 | −0.396 | 2.634 |
| ATOM | 29 | C50 | URI | 1 | 1.343 | −7.291 | 0.360 | −0.34 | +0.00 | +0.000 | 2.634 |
| ATOM | 30 | C44 | URI | 1 | 1.562 | −3.644 | −2.552 | −0.24 | +0.08 | +0.113 | 2.634 |
| ATOM | 31 | O47 | URI | 1 | 0.704 | −3.180 | −1.514 | +0.16 | −0.23 | −0.537 | 2.634 |
| ATOM | 32 | H47 | URI | 1 | 0.121 | −2.491 | −1.898 | +0.11 | +0.26 | +0.424 | 2.634 |
| ATOM | 33 | C43 | URI | 1 | 2.114 | −2.412 | −3.313 | −0.36 | +0.10 | +0.113 | 2.634 |
| ATOM | 34 | O48 | URI | 1 | 1.032 | −1.739 | −3.946 | −0.33 | −1.17 | −0.537 | 2.634 |
| ATOM | 35 | H48 | URI | 1 | 1.255 | −0.790 | −3.876 | +0.06 | +0.61 | +0.424 | 2.634 |
| ATOM | 36 | C46 | URI | 1 | 3.177 | −2.839 | −4.363 | −0.45 | +0.11 | +0.113 | 2.634 |
| ATOM | 37 | C42 | URI | 1 | 3.876 | −1.652 | −5.044 | −0.63 | +0.13 | +0.113 | 2.634 |
| ATOM | 38 | O39 | URI | 1 | 4.172 | −1.982 | −6.397 | −0.16 | −0.81 | −0.537 | 2.634 |
| ATOM | 39 | H39 | URI | 1 | 3.324 | −2.209 | −6.844 | +0.06 | +0.56 | +0.424 | 2.634 |
| ATOM | 40 | O40 | URI | 1 | 4.200 | −3.657 | −3.719 | +0.13 | −0.27 | −0.227 | 2.634 |
| ATOM | 41 | C12 | URI | 1 | −1.739 | −7.065 | −5.647 | −0.38 | +0.04 | +0.113 | 2.634 |
| ATOM | 42 | O15 | URI | 1 | −2.712 | −6.662 | −6.617 | −0.21 | −0.43 | −0.537 | 2.634 |
| ATOM | 43 | H15 | URI | 1 | −2.466 | −5.751 | −6.861 | −0.08 | −0.15 | +0.424 | 2.634 |
| ATOM | 44 | C10 | URI | 1 | −2.300 | −8.318 | −4.938 | −0.33 | +0.07 | +0.113 | 2.634 |
| ATOM | 45 | O11 | URI | 1 | −3.703 | −8.230 | −4.844 | −0.09 | −0.53 | −0.537 | 2.634 |
| ATOM | 46 | H11 | URI | 1 | −4.029 | −8.632 | −5.676 | +0.07 | +0.53 | +0.424 | 2.634 |
| TER | | | | | | | | | | | |
| ENDMDL | | | | | | | | | | | |
| MODEL | 31 | | | | | | | | | | |
| USER | Run = 31 | | | | | | | | | | |
| USER | Cluster Rank = 2 | | | | | | | | | | |
| USER | Number of conformations in this cluster = 1 | | | | | | | | | | |
| USER | | | | | | | | | | | |
| USER | RMSD from reference structure = 2.687 A | | | | | | | | | | |
| USER | | | | | | | | | | | |
| USER | Estimated Free Energy of Binding = −8.03 kcal/mol [= (1) + (3)] | | | | | | | | | | |
| USER | Estimated Inhibition Constant, Ki = +1.29e−06 [Temperature = 298.15 K] | | | | | | | | | | |
| USER | | | | | | | | | | | |
| USER | Final Docked Energy = −11.18 kcal/mol [= (1) + (2)] | | | | | | | | | | |
| USER | | | | | | | | | | | |
| USER | (1) Final Intermolecular Energy = −12.70 kcal/mol | | | | | | | | | | |
| USER | (2) Final Internal Energy of Ligand = +1.52 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.559303 −4.038746 −5.204860 | | | | | | | | | | |
| USER | NEWDPF quat0 0.800329 0.386363 0.458473 −7.718583 | | | | | | | | | | |
| USER | NEWDPF ndihe 15 | | | | | | | | | | |
| USER | NEWDPF dihe0 132.17 28.44 114.10 38.17 −172.65 179.25 1.60 23.84 −28.82 | | | | | | | | | | |
| | 179.95 −95.75 −88.86 −77.69 −119.86 9.41 | | | | | | | | | | |
| USER | | | | | | | | | | | |
| USER | | | | | x | y | z | vdW | Elec | q | RMS |
| ATOM | 1 | C2 | URI | 1 | −1.442 | −11.845 | −6.135 | −0.64 | +0.24 | +0.296 | 2.687 |
| ATOM | 2 | N3 | URI | 1 | −1.476 | −12.964 | −5.341 | −0.40 | −0.20 | −0.292 | 2.687 |
| ATOM | 3 | C4 | URI | 1 | −1.415 | −12.988 | −3.946 | −0.66 | +0.24 | +0.346 | 2.687 |
| ATOM | 4 | C5 | URI | 1 | −1.293 | −11.702 | −3.334 | −0.46 | +0.00 | +0.000 | 2.687 |
| ATOM | 5 | C6 | URI | 1 | −1.264 | −10.603 | −4.102 | −0.27 | −0.03 | −0.050 | 2.687 |
| ATOM | 6 | N1 | URI | 1 | −1.318 | −10.646 | −5.491 | −0.33 | +0.03 | +0.039 | 2.687 |
| ATOM | 7 | O8 | URI | 1 | −1.450 | −14.081 | −3.382 | −0.15 | −0.30 | −0.396 | 2.687 |
| ATOM | 8 | O7 | URI | 1 | −1.507 | −11.921 | −7.362 | −0.25 | −0.26 | −0.396 | 2.687 |
| ATOM | 9 | H3 | URI | 1 | −1.551 | −13.849 | −5.808 | −0.19 | +0.09 | +0.393 | 2.687 |
| ATOM | 10 | C9 | URI | 1 | −1.260 | −9.411 | −6.281 | −0.50 | +0.10 | +0.174 | 2.687 |
| ATOM | 11 | O14 | URI | 1 | 0.073 | −8.927 | −6.171 | −0.04 | −0.12 | −0.227 | 2.687 |
| ATOM | 12 | C13 | URI | 1 | 0.090 | −7.502 | −6.456 | −0.46 | +0.04 | +0.113 | 2.687 |
| ATOM | 13 | C16 | URI | 1 | 0.994 | −6.811 | −5.434 | −0.38 | +0.04 | +0.113 | 2.687 |
| ATOM | 14 | O17 | URI | 1 | 2.280 | −7.393 | −5.530 | −0.07 | −0.20 | −0.368 | 2.687 |
| ATOM | 15 | P18 | URI | 1 | 3.739 | −6.794 | −5.588 | −0.65 | +1.02 | +1.210 | 2.687 |
| ATOM | 16 | O19 | URI | 1 | 4.666 | −7.793 | −4.987 | −0.29 | −0.83 | −0.850 | 2.687 |
| ATOM | 17 | O20 | URI | 1 | 4.006 | −6.331 | −6.987 | −0.04 | −0.85 | −0.850 | 2.687 |
| ATOM | 18 | O21 | URI | 1 | 3.465 | −5.616 | −4.549 | +0.01 | −0.45 | −0.510 | 2.687 |
| ATOM | 19 | P22 | URI | 1 | 2.962 | −5.592 | −3.031 | −0.44 | +0.90 | +1.210 | 2.687 |
| ATOM | 20 | O25 | URI | 1 | 2.583 | −6.921 | −2.488 | +0.07 | −0.43 | −0.850 | 2.687 |
| ATOM | 21 | O24 | URI | 1 | 4.042 | −4.801 | −2.390 | −0.06 | −1.00 | −0.850 | 2.687 |
| ATOM | 22 | O23 | URI | 1 | 1.571 | −4.772 | −3.074 | +0.13 | −0.22 | −0.368 | 2.687 |
| ATOM | 23 | C37 | URI | 1 | 1.656 | −4.008 | −1.892 | −0.22 | +0.12 | +0.227 | 2.687 |
| ATOM | 24 | C45 | URI | 1 | 0.292 | −3.990 | −1.135 | −0.14 | +0.06 | +0.211 | 2.687 |
| ATOM | 25 | N41 | URI | 1 | −0.167 | −5.389 | −0.910 | −0.06 | −0.14 | −0.650 | 2.687 |
| ATOM | 26 | H41 | URI | 1 | 0.221 | −6.078 | −1.555 | +0.11 | +0.12 | +0.440 | 2.687 |
| ATOM | 27 | C49 | URI | 1 | −0.952 | −5.752 | 0.109 | −0.10 | +0.04 | +0.396 | 2.687 |
| ATOM | 28 | O38 | URI | 1 | −1.428 | −4.964 | 0.927 | +0.20 | +0.01 | −0.396 | 2.687 |
| ATOM | 29 | C50 | URI | 1 | −1.241 | −7.246 | 0.219 | −0.13 | +0.00 | +0.000 | 2.687 |
| ATOM | 30 | C44 | URI | 1 | −0.748 | −3.126 | −1.914 | −0.26 | +0.05 | +0.113 | 2.687 |
| ATOM | 31 | O47 | URI | 1 | −1.929 | −2.954 | −1.135 | +0.10 | +0.09 | −0.537 | 2.687 |
| ATOM | 32 | H47 | URI | 1 | −2.028 | −1.995 | −0.955 | −0.17 | −0.16 | +0.424 | 2.687 |
| ATOM | 33 | C43 | URI | 1 | −0.200 | −1.724 | −2.282 | −0.34 | +0.10 | +0.113 | 2.687 |
| ATOM | 34 | O48 | URI | 1 | −1.147 | −1.064 | −3.113 | −0.37 | −0.82 | −0.537 | 2.687 |
| ATOM | 35 | H48 | URI | 1 | −1.893 | −0.849 | −2.518 | −0.02 | +0.11 | +0.424 | 2.687 |
| ATOM | 36 | C46 | URI | 1 | 1.171 | −1.837 | −3.003 | −0.35 | +0.13 | +0.113 | 2.687 |
| ATOM | 37 | C42 | URI | 1 | 1.842 | −0.478 | −3.257 | −0.49 | +0.08 | +0.113 | 2.687 |
| ATOM | 38 | O39 | URI | 1 | 2.097 | −0.321 | −4.649 | −0.35 | −0.72 | −0.537 | 2.687 |
| ATOM | 39 | H39 | URI | 1 | 2.767 | −0.993 | −4.910 | +0.06 | +0.51 | +0.424 | 2.687 |
| ATOM | 40 | O40 | URI | 1 | 2.082 | −2.647 | −2.201 | +0.10 | −0.13 | −0.227 | 2.687 |
| ATOM | 41 | C12 | URI | 1 | −1.367 | −7.050 | −6.368 | −0.37 | +0.02 | +0.113 | 2.687 |
| ATOM | 42 | O15 | URI | 1 | −1.908 | −6.907 | −7.686 | −0.13 | −0.12 | −0.537 | 2.687 |
| ATOM | 43 | H15 | URI | 1 | −1.211 | −6.458 | −8.200 | −0.16 | −0.03 | +0.424 | 2.687 |
| ATOM | 44 | C10 | URI | 1 | −2.085 | −8.253 | −5.714 | −0.39 | +0.07 | +0.113 | 2.687 |
| ATOM | 45 | O11 | URI | 1 | −3.416 | −8.324 | −6.170 | −0.23 | −0.66 | −0.537 | 2.687 |
| ATOM | 46 | H11 | URI | 1 | −3.914 | −8.695 | −5.413 | +0.08 | +0.48 | +0.424 | 2.687 |
| TER | | | | | | | | | | | |
| ENDMDL | | | | | | | | | | | |
| MODEL | 5 | | | | | | | | | | |
| USER | Run = 5 | | | | | | | | | | |
| USER | Cluster Rank = 3 | | | | | | | | | | |
| USER | Number of conformations in this cluster = 1 | | | | | | | | | | |
| USER | | | | | | | | | | | |
| USER | RMSD from reference structure = 2.450 A | | | | | | | | | | |
| USER | | | | | | | | | | | |
| USER | Estimated Free Energy of Binding = −9.04 kcal/mol [= (1) + (3)] | | | | | | | | | | |
| USER | Estimated Inhibition Constant, Ki = +2.37e−07 [Temperature = 298.15 K] | | | | | | | | | | |
| USER | | | | | | | | | | | |
| USER | Final Docked Energy = −11.15 kcal/mol [= (1) + (2)] | | | | | | | | | | |
| USER | | | | | | | | | | | |
| USER | (1) Final Intermolecular Energy = −13.71 kcal/mol | | | | | | | | | | |
| USER | (2) Final Internal Energy of Ligand = +2.56 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 0.430595 −4.211854 −2.602465 | | | | | | | | | | |
| USER | NEWDPF quat0 −0.328266 0.943878 −0.036541 −20.430492 | | | | | | | | | | |
| USER | NEWDPF ndihe 15 | | | | | | | | | | |
| USER | NEWDPF dihe0 −170.85 161.31 −96.28 111.81 −31.46 179.73 127.64 24.59 −54.25 | | | | | | | | | | |
| | −133.58 −67.41 −10.09 40.58 −77.37 −11.69 | | | | | | | | | | |
| USER | | | | | | | | | | | |
| USER | | | | | x | y | z | vdW | Elec | q | RMS |
| ATOM | 1 | C2 | URI | 1 | −1.220 | −11.791 | −5.864 | −0.67 | +0.25 | +0.296 | 2.450 |
| ATOM | 2 | N3 | URI | 1 | −1.329 | −13.058 | −5.347 | −0.41 | −0.20 | −0.292 | 2.450 |
| ATOM | 3 | C4 | URI | 1 | −1.646 | −13.387 | −4.028 | −0.65 | +0.25 | +0.346 | 2.450 |
| ATOM | 4 | C5 | URI | 1 | −1.854 | −12.263 | −3.169 | −0.50 | +0.00 | +0.000 | 2.450 |
| ATOM | 5 | C6 | URI | 1 | −1.754 | −11.021 | −3.666 | −0.42 | −0.03 | −0.050 | 2.450 |
| ATOM | 6 | N1 | URI | 1 | −1.424 | −10.759 | −4.992 | −0.31 | +0.03 | +0.039 | 2.450 |
| ATOM | 7 | O8 | URI | 1 | −1.698 | −14.578 | −3.723 | −0.19 | −0.32 | −0.396 | 2.450 |
| ATOM | 8 | O7 | URI | 1 | −0.940 | −11.597 | −7.048 | −0.27 | −0.38 | −0.396 | 2.450 |
| ATOM | 9 | H3 | URI | 1 | −1.166 | −13.821 | −5.979 | −0.23 | +0.16 | +0.393 | 2.450 |
| ATOM | 10 | C9 | URI | 1 | −1.307 | −9.378 | −5.475 | −0.43 | +0.10 | +0.174 | 2.450 |
| ATOM | 11 | O14 | URI | 1 | −0.462 | −8.701 | −4.552 | +0.08 | −0.11 | −0.227 | 2.450 |
| ATOM | 12 | C13 | URI | 1 | −0.708 | −7.272 | −4.638 | −0.30 | +0.04 | +0.113 | 2.450 |
| ATOM | 13 | C16 | URI | 1 | −0.759 | −6.701 | −3.221 | −0.22 | +0.03 | +0.113 | 2.450 |
| ATOM | 14 | O17 | URI | 1 | 0.489 | −6.090 | −2.954 | +0.13 | −0.14 | −0.368 | 2.450 |
| ATOM | 15 | P18 | URI | 1 | 1.714 | −6.482 | −2.040 | −0.34 | +0.49 | +1.210 | 2.450 |
| ATOM | 16 | O19 | URI | 1 | 2.068 | −5.291 | −1.217 | +0.10 | −0.39 | −0.850 | 2.450 |
| ATOM | 17 | O20 | URI | 1 | 1.387 | −7.760 | −1.331 | +0.03 | −0.18 | −0.850 | 2.450 |
| ATOM | 18 | O21 | URI | 1 | 2.756 | −6.643 | −3.235 | −0.01 | −0.32 | −0.510 | 2.450 |
| ATOM | 19 | P22 | URI | 1 | 3.701 | −5.610 | −4.009 | −0.56 | +1.15 | +1.210 | 2.450 |
| ATOM | 20 | O25 | URI | 1 | 4.377 | −4.620 | −3.133 | −0.01 | −1.10 | −0.850 | 2.450 |
| ATOM | 21 | O24 | URI | 1 | 4.501 | −6.536 | −4.849 | −0.15 | −0.94 | −0.850 | 2.450 |
| ATOM | 22 | O23 | URI | 1 | 2.691 | −4.721 | −4.902 | +0.09 | −0.29 | −0.368 | 2.450 |
| ATOM | 23 | C37 | URI | 1 | 3.583 | −4.079 | −5.785 | −0.50 | +0.26 | +0.227 | 2.450 |
| ATOM | 24 | C45 | URI | 1 | 3.219 | −2.571 | −5.953 | −0.58 | +0.25 | +0.211 | 2.450 |
| ATOM | 25 | N41 | URI | 1 | 3.138 | −1.929 | −4.612 | −0.36 | −0.68 | −0.650 | 2.450 |
| ATOM | 26 | H41 | URI | 1 | 3.943 | −1.357 | −4.360 | +0.05 | +0.44 | +0.440 | 2.450 |
| ATOM | 27 | C49 | URI | 1 | 2.063 | −2.007 | −3.821 | −0.45 | +0.43 | +0.396 | 2.450 |
| ATOM | 28 | O38 | URI | 1 | 1.057 | −2.667 | −4.088 | −0.27 | −0.76 | −0.396 | 2.450 |
| ATOM | 29 | C50 | URI | 1 | 2.139 | −1.209 | −2.523 | −0.41 | +0.00 | +0.000 | 2.450 |
| ATOM | 30 | C44 | URI | 1 | 1.904 | −2.421 | −6.779 | −0.61 | +0.12 | +0.113 | 2.450 |
| ATOM | 31 | O47 | URI | 1 | 1.671 | −1.051 | −7.094 | −0.17 | −0.93 | −0.537 | 2.450 |
| ATOM | 32 | H47 | URI | 1 | 0.891 | −1.011 | −7.687 | +0.02 | +0.60 | +0.424 | 2.450 |
| ATOM | 33 | C43 | URI | 1 | 1.939 | −3.224 | −8.104 | −0.65 | +0.08 | +0.113 | 2.450 |
| ATOM | 34 | O48 | URI | 1 | 0.658 | −3.156 | −8.718 | −0.18 | −0.16 | −0.537 | 2.450 |
| ATOM | 35 | H48 | URI | 1 | 0.614 | −3.949 | −9.290 | +0.03 | +0.10 | +0.424 | 2.450 |
| ATOM | 36 | C46 | URI | 1 | 2.340 | −4.703 | −7.845 | −0.58 | +0.07 | +0.113 | 2.450 |
| ATOM | 37 | C42 | URI | 1 | 2.544 | −5.517 | −9.133 | −0.65 | +0.08 | +0.113 | 2.450 |
| ATOM | 38 | O39 | URI | 1 | 1.676 | −6.645 | −9.130 | −0.12 | −0.35 | −0.537 | 2.450 |
| ATOM | 39 | H39 | URI | 1 | 0.797 | −6.351 | −9.462 | +0.05 | +0.20 | +0.424 | 2.450 |
| ATOM | 40 | O40 | URI | 1 | 3.582 | −4.748 | −7.081 | −0.04 | −0.25 | −0.227 | 2.450 |
| ATOM | 41 | C12 | URI | 1 | −2.032 | −7.130 | −5.388 | −0.37 | +0.05 | +0.113 | 2.450 |
| ATOM | 42 | O15 | URI | 1 | −1.779 | −6.768 | −6.749 | −0.03 | −0.07 | −0.537 | 2.450 |
| ATOM | 43 | H15 | URI | 1 | −0.918 | −7.173 | −6.965 | +0.04 | +0.07 | +0.424 | 2.450 |
| ATOM | 44 | C10 | URI | 1 | −2.603 | −8.567 | −5.402 | −0.38 | +0.08 | +0.113 | 2.450 |
| ATOM | 45 | O11 | URI | 1 | −3.378 | −8.765 | −6.562 | −0.22 | −0.53 | −0.537 | 2.450 |
| ATOM | 46 | H11 | URI | 1 | −3.424 | −7.880 | −6.979 | +0.07 | +0.60 | +0.424 | 2.450 |
| TER | | | | | | | | | | | |
| ENDMDL | | | | | | | | | | | |
| MODEL | 33 | | | | | | | | | | |
| USER | Run = 33 | | | | | | | | | | |
| USER | Cluster Rank = 4 | | | | | | | | | | |
| USER | Number of conformations in this cluster = 1 | | | | | | | | | | |
| USER | | | | | | | | | | | |
| USER | RMSD from reference structure = 3.263 A | | | | | | | | | | |
| USER | | | | | | | | | | | |
| USER | Estimated Free Energy of Binding = −9.64 kcal/mol [= (1) + (3)] | | | | | | | | | | |
| USER | Estimated Inhibition Constant, Ki = +8.65e−08 [Temperature = 298.15 K] | | | | | | | | | | |
| USER | | | | | | | | | | | |
| USER | Final Docked Energy = −11.01 kcal/mol [= (1) + (2)] | | | | | | | | | | |
| USER | | | | | | | | | | | |
| USER | (1) Final Intermolecular Energy = −14.31 kcal/mol | | | | | | | | | | |
| USER | (2) Final Internal Energy of Ligand = +3.29 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 4.590098 −2.756369 −5.137102 | | | | | | | | | | |
| USER | NEWDPF quat0 −0.210769 −0.830206 −0.516076 45.524502 | | | | | | | | | | |
| USER | NEWDPF ndihe 15 | | | | | | | | | | |
| USER | NEWDPF dihe0 −174.82 58.13 75.01 1.22 147.75 178.69 126.18 46.98 21.95 | | | | | | | | | | |
| | 175.91 −179.99 −180.00 1.25 1.77 −4.63 | | | | | | | | | | |
| USER | | | | | | | | | | | |
| USER | | | | | x | y | z | vdW | Elec | q | RMS |
| ATOM | 1 | C2 | URI | 1 | 0.359 | −9.506 | −7.850 | −0.60 | +0.22 | +0.296 | 3.263 |
| ATOM | 2 | N3 | URI | 1 | −0.464 | −10.428 | −7.255 | −0.49 | −0.23 | −0.292 | 3.263 |
| ATOM | 3 | C4 | URI | 1 | −1.207 | −10.240 | −6.088 | −0.56 | +0.24 | +0.346 | 3.263 |
| ATOM | 4 | C5 | URI | 1 | −1.053 | −8.953 | −5.486 | −0.41 | +0.00 | +0.000 | 3.263 |
| ATOM | 5 | C6 | URI | 1 | −0.254 | −8.043 | −6.063 | −0.45 | −0.02 | −0.050 | 3.263 |
| ATOM | 6 | N1 | URI | 1 | 0.470 | −8.296 | −7.223 | −0.39 | +0.02 | +0.039 | 3.263 |
| ATOM | 7 | O8 | URI | 1 | −1.894 | −11.176 | −5.680 | −0.23 | −0.34 | −0.396 | 3.263 |
| ATOM | 8 | O7 | URI | 1 | 0.974 | −9.760 | −8.886 | +0.30 | −0.44 | −0.396 | 3.263 |
| ATOM | 9 | H3 | URI | 1 | −0.538 | −11.324 | −7.701 | +0.04 | +0.41 | +0.393 | 3.263 |
| ATOM | 10 | C9 | URI | 1 | 1.346 | −7.268 | −7.798 | −0.49 | +0.08 | +0.174 | 3.263 |
| ATOM | 11 | O14 | URI | 1 | 2.270 | −6.913 | −6.777 | −0.01 | −0.12 | −0.227 | 3.263 |
| ATOM | 12 | C13 | URI | 1 | 2.805 | −5.590 | −7.054 | −0.52 | +0.08 | +0.113 | 3.263 |
| ATOM | 13 | C16 | URI | 1 | 2.843 | −4.797 | −5.747 | −0.40 | +0.09 | +0.113 | 3.263 |
| ATOM | 14 | O17 | URI | 1 | 3.370 | −3.516 | −6.033 | +0.00 | −0.42 | −0.368 | 3.263 |
| ATOM | 15 | P18 | URI | 1 | 4.263 | −2.504 | −5.215 | −0.66 | +1.65 | +1.210 | 3.263 |
| ATOM | 16 | O19 | URI | 1 | 5.547 | −3.189 | −4.891 | −0.30 | −1.71 | −0.850 | 3.263 |
| ATOM | 17 | O20 | URI | 1 | 4.321 | −1.209 | −5.965 | −0.09 | −1.19 | −0.850 | 3.263 |
| ATOM | 18 | O21 | URI | 1 | 3.325 | −2.475 | −3.926 | +0.01 | −0.47 | −0.510 | 3.263 |
| ATOM | 19 | P22 | URI | 1 | 1.761 | −2.726 | −3.706 | −0.34 | +1.34 | +1.210 | 3.263 |
| ATOM | 20 | O25 | URI | 1 | 0.977 | −2.855 | −4.960 | −0.25 | −1.59 | −0.850 | 3.263 |
| ATOM | 21 | O24 | URI | 1 | 1.792 | −3.839 | −2.724 | +0.12 | −0.57 | −0.850 | 3.263 |
| ATOM | 22 | O23 | URI | 1 | 1.217 | −1.360 | −3.039 | −0.14 | −0.41 | −0.368 | 3.263 |
| ATOM | 23 | C37 | URI | 1 | 1.381 | −1.630 | −1.665 | −0.28 | +0.10 | +0.227 | 3.263 |
| ATOM | 24 | C45 | URI | 1 | 0.003 | −1.708 | −0.938 | −0.25 | +0.04 | +0.211 | 3.263 |
| ATOM | 25 | N41 | URI | 1 | −0.872 | −2.687 | −1.639 | −0.17 | −0.22 | −0.650 | 3.263 |
| ATOM | 26 | H41 | URI | 1 | −0.942 | −3.600 | −1.190 | +0.11 | +0.08 | +0.440 | 3.263 |
| ATOM | 27 | C49 | URI | 1 | −1.593 | −2.387 | −2.724 | −0.31 | +0.32 | +0.396 | 3.263 |
| ATOM | 28 | O38 | URI | 1 | −1.561 | −1.294 | −3.292 | −0.42 | −0.59 | −0.396 | 3.263 |
| ATOM | 29 | C50 | URI | 1 | −2.495 | −3.501 | −3.246 | −0.37 | +0.00 | +0.000 | 3.263 |
| ATOM | 30 | C44 | URI | 1 | −0.635 | −0.287 | −0.836 | −0.39 | +0.00 | +0.113 | 3.263 |
| ATOM | 31 | O47 | URI | 1 | −1.797 | −0.325 | −0.011 | −0.05 | +0.24 | −0.537 | 3.263 |
| ATOM | 32 | H47 | URI | 1 | −2.075 | 0.601 | 0.152 | +0.07 | −0.23 | +0.424 | 3.263 |
| ATOM | 33 | C43 | URI | 1 | 0.342 | 0.763 | −0.248 | −0.33 | −0.02 | +0.113 | 3.263 |
| ATOM | 34 | O48 | URI | 1 | −0.261 | 2.049 | −0.329 | +0.01 | +0.19 | −0.537 | 3.263 |
| ATOM | 35 | H48 | URI | 1 | −0.597 | 2.222 | 0.573 | +0.10 | −0.25 | +0.424 | 3.263 |
| ATOM | 36 | C46 | URI | 1 | 1.696 | 0.748 | −1.012 | −0.41 | +0.00 | +0.113 | 3.263 |
| ATOM | 37 | C42 | URI | 1 | 2.763 | 1.656 | −0.382 | −0.49 | −0.02 | +0.113 | 3.263 |
| ATOM | 38 | O39 | URI | 1 | 3.227 | 2.593 | −1.348 | −0.09 | +0.07 | −0.537 | 3.263 |
| ATOM | 39 | H39 | URI | 1 | 4.121 | 2.304 | −1.643 | −0.28 | −0.18 | +0.424 | 3.263 |
| ATOM | 40 | O40 | URI | 1 | 2.224 | −0.612 | −1.047 | +0.08 | −0.02 | −0.227 | 3.263 |
| ATOM | 41 | C12 | URI | 1 | 1.867 | −4.982 | −8.096 | −0.59 | +0.05 | +0.113 | 3.263 |
| ATOM | 42 | O15 | URI | 1 | 2.469 | −5.064 | −9.392 | −0.17 | −0.37 | −0.537 | 3.263 |
| ATOM | 43 | H15 | URI | 1 | 3.101 | −4.322 | −9.428 | +0.04 | +0.35 | +0.424 | 3.263 |
| ATOM | 44 | C10 | URI | 1 | 0.653 | −5.938 | −8.101 | −0.41 | +0.01 | +0.113 | 3.263 |
| ATOM | 45 | O11 | URI | 1 | 0.067 | −5.965 | −9.382 | −0.04 | −0.13 | −0.537 | 3.263 |
| ATOM | 46 | H11 | URI | 1 | −0.602 | −6.678 | −9.330 | +0.05 | +0.12 | +0.424 | 3.263 |
| TER | | | | | | | | | | | |
| ENDMDL | | | | | | | | | | | |
| MODEL | 38 | | | | | | | | | | |
| USER | Run = 38 | | | | | | | | | | |
| USER | Cluster Rank = 5 | | | | | | | | | | |
| USER | Number of conformations in this cluster = 2 | | | | | | | | | | |
| USER | | | | | | | | | | | |
| USER | RMSD from reference structure = 2.239 A | | | | | | | | | | |
| USER | | | | | | | | | | | |
| USER | Estimated Free Energy of Binding = −7.90 kcal/mol [= (1) + (3)] | | | | | | | | | | |
| USER | Estimated Inhibition Constant, Ki = +1.63e−06 [Temperature = 298.15 K] | | | | | | | | | | |
| USER | | | | | | | | | | | |
| USER | Final Docked Energy = −10.99 kcal/mol [= (1) + (2)] | | | | | | | | | | |
| USER | | | | | | | | | | | |
| USER | (1) Final Intermolecular Energy = −12.57 kcal/mol | | | | | | | | | | |
| USER | (2) Final Internal Energy of Ligand = +1.58 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 0.595786 −3.729313 −4.138938 | | | | | | | | | | |
| USER | NEWDPF quat0 0.558811 0.351350 0.751188 4.244783 | | | | | | | | | | |
| USER | NEWDPF ndihe 15 | | | | | | | | | | |
| USER | NEWDPF dihe0 176.73 32.66 159.58 34.03 −179.83 −165.22 −60.39 −16.68 −2.69 | | | | | | | | | | |
| | 158.61 92.79 −135.05 −61.02 −79.89 22.96 | | | | | | | | | | |
| USER | | | | | | | | | | | |
| USER | | | | | x | y | z | vdW | Elec | q | RMS |
| ATOM | 1 | C2 | URI | 1 | −1.596 | −11.630 | −6.032 | −0.64 | +0.24 | +0.296 | 2.239 |
| ATOM | 2 | N3 | URI | 1 | −1.436 | −12.846 | −5.417 | −0.40 | −0.21 | −0.292 | 2.239 |
| ATOM | 3 | C4 | URI | 1 | −1.251 | −13.062 | −4.050 | −0.70 | +0.24 | +0.346 | 2.239 |
| ATOM | 4 | C5 | URI | 1 | −1.218 | −11.872 | −3.260 | −0.50 | +0.00 | +0.000 | 2.239 |
| ATOM | 5 | C6 | URI | 1 | −1.378 | −10.679 | −3.851 | −0.27 | −0.03 | −0.050 | 2.239 |
| ATOM | 6 | N1 | URI | 1 | −1.548 | −10.529 | −5.223 | −0.30 | +0.03 | +0.039 | 2.239 |
| ATOM | 7 | O8 | URI | 1 | −1.115 | −14.221 | −3.658 | −0.23 | −0.31 | −0.396 | 2.239 |
| ATOM | 8 | O7 | URI | 1 | −1.760 | −11.537 | −7.249 | −0.23 | −0.27 | −0.396 | 2.239 |
| ATOM | 9 | H3 | URI | 1 | −1.454 | −13.658 | −6.007 | −0.24 | +0.09 | +0.393 | 2.239 |
| ATOM | 10 | C9 | URI | 1 | −1.697 | −9.196 | −5.819 | −0.43 | +0.10 | +0.174 | 2.239 |
| ATOM | 11 | O14 | URI | 1 | −0.380 | −8.700 | −6.024 | +0.01 | −0.11 | −0.227 | 2.239 |
| ATOM | 12 | C13 | URI | 1 | −0.421 | −7.250 | −6.102 | −0.39 | +0.03 | +0.113 | 2.239 |
| ATOM | 13 | C16 | URI | 1 | 0.746 | −6.685 | −5.292 | −0.35 | +0.04 | +0.113 | 2.239 |
| ATOM | 14 | O17 | URI | 1 | 1.795 | −6.385 | −6.193 | +0.04 | −0.17 | −0.368 | 2.239 |
| ATOM | 15 | P18 | URI | 1 | 2.959 | −5.320 | −6.186 | −0.44 | +0.97 | +1.210 | 2.239 |
| ATOM | 16 | O19 | URI | 1 | 4.225 | −6.031 | −5.849 | −0.07 | −0.95 | −0.850 | 2.239 |
| ATOM | 17 | O20 | URI | 1 | 2.901 | −4.544 | −7.465 | −0.02 | −0.72 | −0.850 | 2.239 |
| ATOM | 18 | O21 | URI | 1 | 2.431 | −4.521 | −4.910 | +0.10 | −0.39 | −0.510 | 2.239 |
| ATOM | 19 | P22 | URI | 1 | 1.856 | −3.044 | −4.707 | −0.38 | +1.37 | +1.210 | 2.239 |
| ATOM | 20 | O25 | URI | 1 | 2.323 | −2.057 | −5.713 | −0.12 | −1.06 | −0.850 | 2.239 |
| ATOM | 21 | O24 | URI | 1 | 0.406 | −3.326 | −4.553 | −0.22 | −1.70 | −0.850 | 2.239 |
| ATOM | 22 | O23 | URI | 1 | 2.512 | −2.535 | −3.322 | +0.05 | −0.31 | −0.368 | 2.239 |
| ATOM | 23 | C37 | URI | 1 | 1.375 | −2.413 | −2.497 | −0.29 | +0.18 | +0.227 | 2.239 |
| ATOM | 24 | C45 | URI | 1 | 1.761 | −1.874 | −1.084 | −0.24 | +0.06 | +0.211 | 2.239 |
| ATOM | 25 | N41 | URI | 1 | 2.532 | −0.609 | −1.231 | −0.19 | −0.04 | −0.650 | 2.239 |
| ATOM | 26 | H41 | URI | 1 | 3.533 | −0.730 | −1.383 | −0.21 | −0.21 | +0.440 | 2.239 |
| ATOM | 27 | C49 | URI | 1 | 1.964 | 0.602 | −1.260 | −0.44 | +0.01 | +0.396 | 2.239 |
| ATOM | 28 | O38 | URI | 1 | 0.758 | 0.801 | −1.103 | +0.05 | +0.00 | −0.396 | 2.239 |
| ATOM | 29 | C50 | URI | 1 | 2.913 | 1.770 | −1.507 | −0.54 | +0.00 | +0.000 | 2.239 |
| ATOM | 30 | C44 | URI | 1 | 2.532 | −2.966 | −0.280 | −0.17 | +0.03 | +0.113 | 2.239 |
| ATOM | 31 | O47 | URI | 1 | 2.719 | −2.547 | 1.069 | +0.14 | −0.09 | −0.537 | 2.239 |
| ATOM | 32 | H47 | URI | 1 | 2.932 | −3.344 | 1.600 | +0.11 | +0.10 | +0.424 | 2.239 |
| ATOM | 33 | C43 | URI | 1 | 1.792 | −4.328 | −0.267 | −0.19 | +0.04 | +0.113 | 2.239 |
| ATOM | 34 | O48 | URI | 1 | 2.627 | −5.300 | 0.352 | +0.09 | −0.23 | −0.537 | 2.239 |
| ATOM | 35 | H48 | URI | 1 | 2.578 | −5.096 | 1.307 | +0.11 | +0.14 | +0.424 | 2.239 |
| ATOM | 36 | C46 | URI | 1 | 1.421 | −4.771 | −1.710 | −0.20 | +0.05 | +0.113 | 2.239 |
| ATOM | 37 | C42 | URI | 1 | 0.551 | −6.036 | −1.757 | −0.22 | +0.04 | +0.113 | 2.239 |
| ATOM | 38 | O39 | URI | 1 | 1.327 | −7.136 | −2.221 | +0.00 | −0.18 | −0.537 | 2.239 |
| ATOM | 39 | H39 | URI | 1 | 2.106 | −6.773 | −2.701 | +0.08 | +0.20 | +0.424 | 2.239 |
| ATOM | 40 | O40 | URI | 1 | 0.694 | −3.698 | −2.380 | +0.13 | −0.15 | −0.227 | 2.239 |
| ATOM | 41 | C12 | URI | 1 | −1.786 | −6.849 | −5.544 | −0.35 | +0.03 | +0.113 | 2.239 |
| ATOM | 42 | O15 | URI | 1 | −2.675 | −6.539 | −6.622 | −0.16 | −0.36 | −0.537 | 2.239 |
| ATOM | 43 | H15 | URI | 1 | −2.167 | −5.952 | −7.211 | −0.16 | −0.19 | +0.424 | 2.239 |
| ATOM | 44 | C10 | URI | 1 | −2.309 | −8.147 | −4.887 | −0.33 | +0.07 | +0.113 | 2.239 |
| ATOM | 45 | O11 | URI | 1 | −3.716 | −8.187 | −4.953 | −0.10 | −0.56 | −0.537 | 2.239 |
| ATOM | 46 | H11 | URI | 1 | −3.979 | −8.769 | −4.211 | +0.09 | +0.36 | +0.424 | 2.239 |
| TER | | | | | | | | | | | |
| ENDMDL | | | | | | | | | | | |
| MODEL | 37 | | | | | | | | | | |
| USER | Run = 37 | | | | | | | | | | |
| USER | Cluster Rank = 5 | | | | | | | | | | |
| USER | Number of conformations in this cluster = 2 | | | | | | | | | | |
| USER | | | | | | | | | | | |
| USER | RMSD from reference structure = 2.432 A | | | | | | | | | | |
| USER | | | | | | | | | | | |
| USER | Estimated Free Energy of Binding = −7.34 kcal/mol [= (1) + (3)] | | | | | | | | | | |
| USER | Estimated Inhibition Constant, Ki = +4.16e−06 [Temperature = 298.15 K] | | | | | | | | | | |
| USER | | | | | | | | | | | |
| USER | Final Docked Energy = −10.58 kcal/mol [= (1) + (2)] | | | | | | | | | | |
| USER | | | | | | | | | | | |
| USER | (1) Final Intermolecular Energy = −12.01 kcal/mol | | | | | | | | | | |
| USER | (2) Final Internal Energy of Ligand = +1.43 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.260240 −4.383989 −4.352814 | | | | | | | | | | |
| USER | NEWDPF quat0 −0.584135 0.573845 −0.574011 1.877497 | | | | | | | | | | |
| USER | NEWDPF ndihe 15 | | | | | | | | | | |
| USER | NEWDPF dihe0 91.27 39.47 −82.09 −1.78 30.95 144.26 −89.01 30.19 −23.00 | | | | | | | | | | |
| | −47.61 −49.98 156.38 −41.68 −87.83 7.75 | | | | | | | | | | |
| USER | | | | | | | | | | | |
| USER | | | | | x | y | z | vdW | Elec | q | RMS |
| ATOM | 1 | C2 | URI | 1 | −1.500 | −12.204 | −5.782 | −0.58 | +0.24 | +0.296 | 2.432 |
| ATOM | 2 | N3 | URI | 1 | −1.433 | −13.388 | −5.092 | −0.39 | −0.19 | −0.292 | 2.432 |
| ATOM | 3 | C4 | URI | 1 | −1.271 | −13.533 | −3.713 | −0.70 | +0.24 | +0.346 | 2.432 |
| ATOM | 4 | C5 | URI | 1 | −1.155 | −12.302 | −2.996 | −0.57 | +0.00 | +0.000 | 2.432 |
| ATOM | 5 | C6 | URI | 1 | −1.223 | −11.139 | −3.661 | −0.26 | −0.03 | −0.050 | 2.432 |
| ATOM | 6 | N1 | URI | 1 | −1.375 | −11.062 | −5.041 | −0.34 | +0.03 | +0.039 | 2.432 |
| ATOM | 7 | O8 | URI | 1 | −1.224 | −14.672 | −3.250 | −0.11 | −0.35 | −0.396 | 2.432 |
| ATOM | 8 | O7 | URI | 1 | −1.650 | −12.174 | −7.004 | −0.19 | −0.24 | −0.396 | 2.432 |
| ATOM | 9 | H3 | URI | 1 | −1.509 | −14.232 | −5.632 | −0.26 | +0.13 | +0.393 | 2.432 |
| ATOM | 10 | C9 | URI | 1 | −1.422 | −9.760 | −5.718 | −0.47 | +0.11 | +0.174 | 2.432 |
| ATOM | 11 | O14 | URI | 1 | −0.113 | −9.212 | −5.621 | −0.01 | −0.13 | −0.227 | 2.432 |
| ATOM | 12 | C13 | URI | 1 | −0.186 | −7.769 | −5.777 | −0.41 | +0.04 | +0.113 | 2.432 |
| ATOM | 13 | C16 | URI | 1 | 0.727 | −7.120 | −4.736 | −0.35 | +0.04 | +0.113 | 2.432 |
| ATOM | 14 | O17 | URI | 1 | 2.010 | −6.975 | −5.315 | −0.01 | −0.19 | −0.368 | 2.432 |
| ATOM | 15 | P18 | URI | 1 | 3.321 | −6.221 | −4.866 | −0.54 | +0.97 | +1.210 | 2.432 |
| ATOM | 16 | O19 | URI | 1 | 3.428 | −6.334 | −3.384 | −0.06 | −0.71 | −0.850 | 2.432 |
| ATOM | 17 | O20 | URI | 1 | 4.463 | −6.718 | −5.699 | −0.15 | −0.89 | −0.850 | 2.432 |
| ATOM | 18 | O21 | URI | 1 | 2.807 | −4.764 | −5.262 | +0.08 | −0.41 | −0.510 | 2.432 |
| ATOM | 19 | P22 | URI | 1 | 2.261 | −3.528 | −4.407 | −0.34 | +1.11 | +1.210 | 2.432 |
| ATOM | 20 | O25 | URI | 1 | 2.945 | −2.240 | −4.682 | −0.02 | −0.90 | −0.850 | 2.432 |
| ATOM | 21 | O24 | URI | 1 | 0.798 | −3.660 | −4.626 | −0.23 | −1.01 | −0.850 | 2.432 |
| ATOM | 22 | O23 | URI | 1 | 2.666 | −3.873 | −2.883 | +0.09 | −0.28 | −0.368 | 2.432 |
| ATOM | 23 | C37 | URI | 1 | 1.475 | −3.562 | −2.195 | −0.23 | +0.14 | +0.227 | 2.432 |
| ATOM | 24 | C45 | URI | 1 | 1.761 | −2.660 | −0.954 | −0.21 | +0.07 | +0.211 | 2.432 |
| ATOM | 25 | N41 | URI | 1 | 2.535 | −1.462 | −1.382 | −0.14 | −0.12 | −0.650 | 2.432 |
| ATOM | 26 | H41 | URI | 1 | 3.486 | −1.411 | −1.018 | −0.14 | −0.17 | +0.440 | 2.432 |
| ATOM | 27 | C49 | URI | 1 | 2.071 | −0.558 | −2.251 | −0.45 | +0.14 | +0.396 | 2.432 |
| ATOM | 28 | O38 | URI | 1 | 0.939 | −0.585 | −2.736 | +0.00 | −0.31 | −0.396 | 2.432 |
| ATOM | 29 | C50 | URI | 1 | 3.047 | 0.552 | −2.631 | −0.50 | +0.00 | +0.000 | 2.432 |
| ATOM | 30 | C44 | URI | 1 | 2.481 | −3.484 | 0.159 | −0.22 | +0.04 | +0.113 | 2.432 |
| ATOM | 31 | O47 | URI | 1 | 2.574 | −2.719 | 1.357 | +0.16 | −0.08 | −0.537 | 2.432 |
| ATOM | 32 | H47 | URI | 1 | 1.690 | −2.725 | 1.782 | +0.11 | +0.02 | +0.424 | 2.432 |
| ATOM | 33 | C43 | URI | 1 | 1.749 | −4.810 | 0.485 | −0.18 | +0.03 | +0.113 | 2.432 |
| ATOM | 34 | O48 | URI | 1 | 2.544 | −5.564 | 1.392 | +0.11 | −0.18 | −0.537 | 2.432 |
| ATOM | 35 | H48 | URI | 1 | 2.343 | −5.186 | 2.271 | +0.11 | +0.10 | +0.424 | 2.432 |
| ATOM | 36 | C46 | URI | 1 | 1.479 | −5.627 | −0.809 | −0.24 | +0.04 | +0.113 | 2.432 |
| ATOM | 37 | C42 | URI | 1 | 0.620 | −6.879 | −0.574 | −0.28 | +0.03 | +0.113 | 2.432 |
| ATOM | 38 | O39 | URI | 1 | 0.834 | −7.813 | −1.627 | +0.00 | −0.13 | −0.537 | 2.432 |
| ATOM | 39 | H39 | URI | 1 | 1.489 | −8.477 | −1.312 | +0.11 | +0.04 | +0.424 | 2.432 |
| ATOM | 40 | O40 | URI | 1 | 0.794 | −4.786 | −1.786 | +0.17 | −0.09 | −0.227 | 2.432 |
| ATOM | 41 | C12 | URI | 1 | −1.660 | −7.412 | −5.586 | −0.38 | +0.05 | +0.113 | 2.432 |
| ATOM | 42 | O15 | URI | 1 | −2.270 | −7.184 | −6.861 | −0.30 | −0.29 | −0.537 | 2.432 |
| ATOM | 43 | H15 | URI | 1 | −2.052 | −6.260 | −7.086 | −0.16 | −0.07 | +0.424 | 2.432 |
| ATOM | 44 | C10 | URI | 1 | −2.281 | −8.707 | −5.014 | −0.35 | +0.07 | +0.113 | 2.432 |
| ATOM | 45 | O11 | URI | 1 | −3.626 | −8.814 | −5.417 | −0.11 | −0.53 | −0.537 | 2.432 |
| ATOM | 46 | H11 | URI | 1 | −3.593 | −8.758 | −6.394 | +0.07 | +0.46 | +0.424 | 2.432 |
| TER | | | | | | | | | | | |
| ENDMDL | | | | | | | | | | | |
| MODEL | 3 | | | | | | | | | | |
| USER | Run = 3 | | | | | | | | | | |
| USER | Cluster Rank = 6 | | | | | | | | | | |
| USER | Number of conformations in this cluster = 2 | | | | | | | | | | |
| USER | | | | | | | | | | | |
| USER | RMSD from reference structure = 3.258 A | | | | | | | | | | |
| USER | | | | | | | | | | | |
| USER | Estimated Free Energy of Binding = −6.96 kcal/mol [= (1) + (3)] | | | | | | | | | | |
| USER | Estimated Inhibition Constant, Ki = +7.89e−06 [Temperature = 298.15 K] | | | | | | | | | | |
| USER | | | | | | | | | | | |
| USER | Final Docked Energy = −10.52 kcal/mol [= (1) + (2)] | | | | | | | | | | |
| USER | | | | | | | | | | | |
| USER | (1) Final Intermolecular Energy = −11.63 kcal/mol | | | | | | | | | | |
| USER | (2) Final Internal Energy of Ligand = +1.11 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −2.176665 −1.312923 −0.615493 | | | | | | | | | | |
| USER | NEWDPF quat0 0.496769 −0.858574 0.126773 28.359330 | | | | | | | | | | |
| USER | NEWDPF ndihe 15 | | | | | | | | | | |
| USER | NEWDPF dihe0 −180.00 35.74 −110.29 75.46 180.00 −133.61 −76.62 −2.60 | | | | | | | | | | |
| | −36.64 5.92 116.52 −5.93 −8.61 −15.00 40.60 | | | | | | | | | | |
| USER | | | | | | | | | | | |
| USER | | | | | x | y | z | vdW | Elec | q | RMS |
| ATOM | 1 | C2 | URI | 1 | −3.002 | −8.524 | −4.873 | −0.35 | +0.22 | +0.296 | 3.258 |
| ATOM | 2 | N3 | URI | 1 | −3.067 | −9.848 | −4.519 | −0.20 | −0.22 | −0.292 | 3.258 |
| ATOM | 3 | C4 | URI | 1 | −3.473 | −10.353 | −3.283 | −0.27 | +0.22 | +0.346 | 3.258 |
| ATOM | 4 | C5 | URI | 1 | −3.833 | −9.357 | −2.323 | −0.17 | +0.00 | +0.000 | 3.258 |
| ATOM | 5 | C6 | URI | 1 | −3.777 | −8.060 | −2.662 | −0.21 | −0.02 | −0.050 | 3.258 |
| ATOM | 6 | N1 | URI | 1 | −3.352 | −7.619 | −3.911 | −0.18 | +0.02 | +0.039 | 3.258 |
| ATOM | 7 | O8 | URI | 1 | −3.468 | −11.573 | −3.124 | +0.09 | −0.27 | −0.396 | 3.258 |
| ATOM | 8 | O7 | URI | 1 | −2.634 | −8.171 | −5.995 | −0.26 | −0.32 | −0.396 | 3.258 |
| ATOM | 9 | H3 | URI | 1 | −2.795 | −10.518 | −5.217 | +0.09 | +0.37 | +0.393 | 3.258 |
| ATOM | 10 | C9 | URI | 1 | −3.291 | −6.186 | −4.218 | −0.29 | +0.07 | +0.174 | 3.258 |
| ATOM | 11 | O14 | URI | 1 | −1.921 | −5.815 | −4.125 | +0.05 | −0.05 | −0.227 | 3.258 |
| ATOM | 12 | C13 | URI | 1 | −1.826 | −4.384 | −3.893 | −0.32 | +0.03 | +0.113 | 3.258 |
| ATOM | 13 | C16 | URI | 1 | −0.761 | −4.133 | −2.825 | −0.26 | +0.06 | +0.113 | 3.258 |
| ATOM | 14 | O17 | URI | 1 | −0.397 | −2.767 | −2.888 | −0.26 | −0.57 | −0.368 | 3.258 |
| ATOM | 15 | P18 | URI | 1 | 0.890 | −1.967 | −2.449 | −0.29 | +1.08 | +1.210 | 3.258 |
| ATOM | 16 | O19 | URI | 1 | 0.453 | −0.854 | −1.559 | +0.08 | −0.29 | −0.850 | 3.258 |
| ATOM | 17 | O20 | URI | 1 | 1.897 | −2.942 | −1.922 | +0.12 | −0.46 | −0.850 | 3.258 |
| ATOM | 18 | O21 | URI | 1 | 1.224 | −1.399 | −3.901 | −0.36 | −0.90 | −0.510 | 3.258 |
| ATOM | 19 | P22 | URI | 1 | 2.110 | −1.949 | −5.113 | −0.58 | +1.60 | +1.210 | 3.258 |
| ATOM | 20 | O25 | URI | 1 | 2.162 | −1.051 | −6.294 | −0.37 | −1.45 | −0.850 | 3.258 |
| ATOM | 21 | O24 | URI | 1 | 3.359 | −2.323 | −4.401 | −0.02 | −0.86 | −0.850 | 3.258 |
| ATOM | 22 | O23 | URI | 1 | 1.316 | −3.252 | −5.642 | −0.13 | −0.36 | −0.368 | 3.258 |
| ATOM | 23 | C37 | URI | 1 | 2.375 | −4.064 | −6.098 | −0.42 | +0.18 | +0.227 | 3.258 |
| ATOM | 24 | C45 | URI | 1 | 1.850 | −5.206 | −7.022 | −0.45 | +0.09 | +0.211 | 3.258 |
| ATOM | 25 | N41 | URI | 1 | 1.032 | −4.619 | −8.118 | −0.22 | −0.06 | −0.650 | 3.258 |
| ATOM | 26 | H41 | URI | 1 | 0.023 | −4.690 | −7.988 | +0.09 | −0.49 | +0.440 | 3.258 |
| ATOM | 27 | C49 | URI | 1 | 1.556 | −3.962 | −9.158 | −0.75 | +0.20 | +0.396 | 3.258 |
| ATOM | 28 | O38 | URI | 1 | 2.766 | −3.830 | −9.351 | −0.11 | −0.32 | −0.396 | 3.258 |
| ATOM | 29 | C50 | URI | 1 | 0.550 | −3.357 | −10.132 | −0.67 | +0.00 | +0.000 | 3.258 |
| ATOM | 30 | C44 | URI | 1 | 1.071 | −6.266 | −6.182 | −0.38 | +0.03 | +0.113 | 3.258 |
| ATOM | 31 | O47 | URI | 1 | 0.755 | −7.399 | −6.986 | −0.03 | −0.19 | −0.537 | 3.258 |
| ATOM | 32 | H47 | URI | 1 | 0.488 | −8.125 | −6.384 | +0.08 | +0.18 | +0.424 | 3.258 |
| ATOM | 33 | C43 | URI | 1 | 1.873 | −6.755 | −4.949 | −0.42 | +0.06 | +0.113 | 3.258 |
| ATOM | 34 | O48 | URI | 1 | 1.039 | −7.597 | −4.162 | +0.05 | −0.24 | −0.537 | 3.258 |
| ATOM | 35 | H48 | URI | 1 | 1.042 | −8.455 | −4.630 | +0.16 | +0.25 | +0.424 | 3.258 |
| ATOM | 36 | C46 | URI | 1 | 2.381 | −5.552 | −4.107 | −0.34 | +0.07 | +0.113 | 3.258 |
| ATOM | 37 | C42 | URI | 1 | 3.314 | −5.962 | −2.956 | −0.52 | +0.09 | +0.113 | 3.258 |
| ATOM | 38 | O39 | URI | 1 | 2.549 | −6.524 | −1.895 | −0.03 | −0.27 | −0.537 | 3.258 |
| ATOM | 39 | H39 | URI | 1 | 2.206 | −7.395 | −2.200 | +0.05 | +0.15 | +0.424 | 3.258 |
| ATOM | 40 | O40 | URI | 1 | 3.107 | −4.622 | −4.967 | +0.07 | −0.20 | −0.227 | 3.258 |
| ATOM | 41 | C12 | URI | 1 | −3.225 | −3.948 | −3.457 | −0.45 | +0.00 | +0.113 | 3.258 |
| ATOM | 42 | O15 | URI | 1 | −3.900 | −3.326 | −4.555 | +0.03 | −0.08 | −0.537 | 3.258 |
| ATOM | 43 | H15 | URI | 1 | −3.234 | −3.269 | −5.265 | +0.03 | −0.20 | +0.424 | 3.258 |
| ATOM | 44 | C10 | URI | 1 | −3.954 | −5.282 | −3.176 | −0.45 | +0.01 | +0.113 | 3.258 |
| ATOM | 45 | O11 | URI | 1 | −5.330 | −5.144 | −3.444 | −0.16 | +0.00 | −0.537 | 3.258 |
| ATOM | 46 | H11 | URI | 1 | −5.757 | −5.831 | −2.892 | +0.07 | +0.03 | +0.424 | 3.258 |
| TER | | | | | | | | | | | |
| ENDMDL | | | | | | | | | | | |
| MODEL | 20 | | | | | | | | | | |
| USER | Run = 20 | | | | | | | | | | |
| USER | Cluster Rank = 6 | | | | | | | | | | |
| USER | Number of conformations in this cluster = 2 | | | | | | | | | | |
| USER | | | | | | | | | | | |
| USER | RMSD from reference structure = 3.718 A | | | | | | | | | | |
| USER | | | | | | | | | | | |
| USER | Estimated Free Energy of Binding = −6.08 kcal/mol [= (1) + (3)] | | | | | | | | | | |
| USER | Estimated Inhibition Constant, Ki = +3.49e−05 [Temperature = 298.15 K] | | | | | | | | | | |
| USER | | | | | | | | | | | |
| USER | Final Docked Energy = −9.20 kcal/mol [= (1) + (2)] | | | | | | | | | | |
| USER | | | | | | | | | | | |
| USER | (1) Final Intermolecular Energy = −10.75 kcal/mol | | | | | | | | | | |
| USER | (2) Final Internal Energy of Ligand = +1.55 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.375337 −2.251698 −1.808440 | | | | | | | | | | |
| USER | NEWDPF quat0 −0.055625 0.125720 −0.990505 24.182573 | | | | | | | | | | |
| USER | NEWDPF ndihe 15 | | | | | | | | | | |
| USER | NEWDPF dihe0 106.16 127.61 110.94 44.57 −158.74 157.62 −75.54 −23.00 −0.70 | | | | | | | | | | |
| | 154.13 −15.99 −89.63 41.08 16.72 0.43 | | | | | | | | | | |
| USER | | | | | | | | | | | |
| USER | | | | | x | y | z | vdW | Elec | q | RMS |
| ATOM | 1 | C2 | URI | 1 | −4.251 | −8.385 | −3.050 | −0.27 | +0.18 | +0.296 | 3.718 |
| ATOM | 2 | N3 | URI | 1 | −4.629 | −9.500 | −2.346 | −0.12 | −0.16 | −0.292 | 3.718 |
| ATOM | 3 | C4 | URI | 1 | −4.492 | −9.694 | −0.970 | −0.11 | +0.12 | +0.346 | 3.718 |
| ATOM | 4 | C5 | URI | 1 | −3.884 | −8.604 | −0.274 | −0.08 | +0.00 | +0.000 | 3.718 |
| ATOM | 5 | C6 | URI | 1 | −3.515 | −7.507 | −0.953 | −0.11 | −0.01 | −0.050 | 3.718 |
| ATOM | 6 | N1 | URI | 1 | −3.669 | −7.379 | −2.329 | −0.10 | +0.01 | +0.039 | 3.718 |
| ATOM | 7 | O8 | URI | 1 | −4.877 | −10.761 | −0.493 | +0.20 | −0.13 | −0.396 | 3.718 |
| ATOM | 8 | O7 | URI | 1 | −4.416 | −8.301 | −4.268 | −0.12 | −0.39 | −0.396 | 3.718 |
| ATOM | 9 | H3 | URI | 1 | −5.043 | −10.249 | −2.872 | +0.10 | +0.31 | +0.393 | 3.718 |
| ATOM | 10 | C9 | URI | 1 | −3.228 | −6.164 | −3.023 | −0.28 | +0.04 | +0.174 | 3.718 |
| ATOM | 11 | O14 | URI | 1 | −1.822 | −6.077 | −2.822 | +0.15 | −0.05 | −0.227 | 3.718 |
| ATOM | 12 | C13 | URI | 1 | −1.397 | −4.698 | −3.000 | −0.27 | +0.04 | +0.113 | 3.718 |
| ATOM | 13 | C16 | URI | 1 | −0.401 | −4.349 | −1.893 | −0.17 | +0.04 | +0.113 | 3.718 |
| ATOM | 14 | O17 | URI | 1 | −0.260 | −2.942 | −1.865 | +0.09 | −0.20 | −0.368 | 3.718 |
| ATOM | 15 | P18 | URI | 1 | 0.892 | −1.959 | −2.311 | −0.29 | +0.97 | +1.210 | 3.718 |
| ATOM | 16 | O19 | URI | 1 | 0.259 | −0.675 | −2.725 | −0.05 | −0.83 | −0.850 | 3.718 |
| ATOM | 17 | O20 | URI | 1 | 1.929 | −1.923 | −1.231 | +0.11 | −0.25 | −0.850 | 3.718 |
| ATOM | 18 | O21 | URI | 1 | 1.317 | −2.793 | −3.602 | −0.16 | −0.67 | −0.510 | 3.718 |
| ATOM | 19 | P22 | URI | 1 | 1.702 | −2.375 | −5.096 | −0.44 | +1.73 | +1.210 | 3.718 |
| ATOM | 20 | O25 | URI | 1 | 1.027 | −1.145 | −5.583 | −0.79 | −1.96 | −0.850 | 3.718 |
| ATOM | 21 | O24 | URI | 1 | 3.183 | −2.441 | −5.034 | −0.02 | −0.93 | −0.850 | 3.718 |
| ATOM | 22 | O23 | URI | 1 | 1.093 | −3.550 | −6.021 | −0.04 | −0.23 | −0.368 | 3.718 |
| ATOM | 23 | C37 | URI | 1 | 2.265 | −4.158 | −6.516 | −0.44 | +0.16 | +0.227 | 3.718 |
| ATOM | 24 | C45 | URI | 1 | 1.926 | −5.418 | −7.371 | −0.50 | +0.10 | +0.211 | 3.718 |
| ATOM | 25 | N41 | URI | 1 | 0.951 | −5.048 | −8.433 | −0.31 | −0.08 | −0.650 | 3.718 |
| ATOM | 26 | H41 | URI | 1 | −0.019 | −5.293 | −8.232 | −0.20 | −0.25 | +0.440 | 3.718 |
| ATOM | 27 | C49 | URI | 1 | 1.283 | −4.362 | −9.532 | −0.75 | +0.17 | +0.396 | 3.718 |
| ATOM | 28 | O38 | URI | 1 | 2.434 | −4.023 | −9.811 | +0.01 | −0.28 | −0.396 | 3.718 |
| ATOM | 29 | C50 | URI | 1 | 0.126 | −4.002 | −10.459 | −0.41 | +0.00 | +0.000 | 3.718 |
| ATOM | 30 | C44 | URI | 1 | 1.418 | −6.574 | −6.454 | −0.45 | +0.04 | +0.113 | 3.718 |
| ATOM | 31 | O47 | URI | 1 | 1.288 | −7.779 | −7.204 | −0.06 | −0.25 | −0.537 | 3.718 |
| ATOM | 32 | H47 | URI | 1 | 2.137 | −8.264 | −7.133 | +0.05 | +0.22 | +0.424 | 3.718 |
| ATOM | 33 | C43 | URI | 1 | 2.366 | −6.846 | −5.259 | −0.49 | +0.06 | +0.113 | 3.718 |
| ATOM | 34 | O48 | URI | 1 | 1.758 | −7.801 | −4.397 | +0.05 | −0.27 | −0.537 | 3.718 |
| ATOM | 35 | H48 | URI | 1 | 2.461 | −8.045 | −3.763 | +0.09 | +0.22 | +0.424 | 3.718 |
| ATOM | 36 | C46 | URI | 1 | 2.672 | −5.533 | −4.485 | −0.37 | +0.08 | +0.113 | 3.718 |
| ATOM | 37 | C42 | URI | 1 | 3.727 | −5.706 | −3.381 | −0.57 | +0.11 | +0.113 | 3.718 |
| ATOM | 38 | O39 | URI | 1 | 3.405 | −4.871 | −2.274 | −0.01 | −0.47 | −0.537 | 3.718 |
| ATOM | 39 | H39 | URI | 1 | 3.538 | −3.936 | −2.553 | +0.10 | +0.40 | +0.424 | 3.718 |
| ATOM | 40 | O40 | URI | 1 | 3.152 | −4.516 | −5.415 | +0.06 | −0.21 | −0.227 | 3.718 |
| ATOM | 41 | C12 | URI | 1 | −2.677 | −3.866 | −2.938 | −0.38 | +0.01 | +0.113 | 3.718 |
| ATOM | 42 | O15 | URI | 1 | −3.078 | −3.498 | −4.262 | +0.00 | −0.02 | −0.537 | 3.718 |
| ATOM | 43 | H15 | URI | 1 | −2.246 | −3.331 | −4.743 | −0.10 | −0.09 | +0.424 | 3.718 |
| ATOM | 44 | C10 | URI | 1 | −3.736 | −4.859 | −2.405 | −0.39 | +0.00 | +0.113 | 3.718 |
| ATOM | 45 | O11 | URI | 1 | −5.005 | −4.529 | −2.919 | −0.16 | +0.07 | −0.537 | 3.718 |
| ATOM | 46 | H11 | URI | 1 | −5.602 | −4.575 | −2.143 | +0.05 | −0.10 | +0.424 | 3.718 |
| TER | | | | | | | | | | | |
| ENDMDL | | | | | | | | | | | |
| MODEL | 48 | | | | | | | | | | |
| USER | Run = 48 | | | | | | | | | | |
| USER | Cluster Rank = 7 | | | | | | | | | | |
| USER | Number of conformations in this cluster = 1 | | | | | | | | | | |
| USER | | | | | | | | | | | |
| USER | RMSD from reference structure = 2.803 A | | | | | | | | | | |
| USER | | | | | | | | | | | |
| USER | Estimated Free Energy of Binding = −7.10 kcal/mol [= (1) + (3)] | | | | | | | | | | |
| USER | Estimated Inhibition Constant, Ki = +6.25e−06 [Temperature = 298.15 K] | | | | | | | | | | |
| USER | | | | | | | | | | | |
| USER | Final Docked Energy = −10.39 kcal/mol [= (1) + (2)] | | | | | | | | | | |
| USER | | | | | | | | | | | |
| USER | (1) Final Intermolecular Energy = −11.77 kcal/mol | | | | | | | | | | |
| USER | (2) Final Internal Energy of Ligand = +1.38 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.734235 −3.854925 −5.487807 | | | | | | | | | | |
| USER | NEWDPF quat0 0.600624 0.720507 0.346585 −14.963886 | | | | | | | | | | |
| USER | NEWDPF ndihe 15 | | | | | | | | | | |
| USER | NEWDPF dihe0 61.40 40.92 115.43 −103.53 35.09 178.17 −73.73 7.54 −10.13 | | | | | | | | | | |
| | 177.18 114.02 −160.77 −4.13 −9.26 −5.27 | | | | | | | | | | |
| USER | | | | | | | | | | | |
| USER | | | | | x | y | z | vdW | Elec | q | RMS |
| ATOM | 1 | C2 | URI | 1 | −1.319 | −11.641 | −6.418 | −0.66 | +0.24 | +0.296 | 2.803 |
| ATOM | 2 | N3 | URI | 1 | −1.489 | −12.714 | −5.579 | −0.41 | −0.21 | −0.292 | 2.803 |
| ATOM | 3 | C4 | URI | 1 | −1.617 | −12.664 | −4.190 | −0.62 | +0.25 | +0.346 | 2.803 |
| ATOM | 4 | C5 | URI | 1 | −1.545 | −11.350 | −3.634 | −0.45 | +0.00 | +0.000 | 2.803 |
| ATOM | 5 | C6 | URI | 1 | −1.383 | −10.294 | −4.446 | −0.36 | −0.03 | −0.050 | 2.803 |
| ATOM | 6 | N1 | URI | 1 | −1.251 | −10.411 | −5.825 | −0.33 | +0.03 | +0.039 | 2.803 |
| ATOM | 7 | O8 | URI | 1 | −1.757 | −13.725 | −3.582 | −0.12 | −0.29 | −0.396 | 2.803 |
| ATOM | 8 | O7 | URI | 1 | −1.220 | −11.782 | −7.637 | −0.24 | −0.29 | −0.396 | 2.803 |
| ATOM | 9 | H3 | URI | 1 | −1.524 | −13.622 | −6.007 | −0.25 | +0.08 | +0.393 | 2.803 |
| ATOM | 10 | C9 | URI | 1 | −1.055 | −9.222 | −6.662 | −0.53 | +0.10 | +0.174 | 2.803 |
| ATOM | 11 | O14 | URI | 1 | 0.100 | −8.564 | −6.157 | −0.02 | −0.11 | −0.227 | 2.803 |
| ATOM | 12 | C13 | URI | 1 | 0.066 | −7.165 | −6.552 | −0.44 | +0.03 | +0.113 | 2.803 |
| ATOM | 13 | C16 | URI | 1 | 0.485 | −6.311 | −5.356 | −0.31 | +0.03 | +0.113 | 2.803 |
| ATOM | 14 | O17 | URI | 1 | 0.700 | −4.991 | −5.820 | +0.20 | −0.03 | −0.368 | 2.803 |
| ATOM | 15 | P18 | URI | 1 | 1.621 | −3.807 | −5.329 | −0.33 | +0.98 | +1.210 | 2.803 |
| ATOM | 16 | O19 | URI | 1 | 3.006 | −4.078 | −5.809 | +0.05 | −0.81 | −0.850 | 2.803 |
| ATOM | 17 | O20 | URI | 1 | 0.977 | −2.513 | −5.720 | −0.37 | −1.34 | −0.850 | 2.803 |
| ATOM | 18 | O21 | URI | 1 | 1.508 | −4.154 | −3.777 | +0.10 | −0.40 | −0.510 | 2.803 |
| ATOM | 19 | P22 | URI | 1 | 0.525 | −3.662 | −2.615 | −0.20 | +0.90 | +1.210 | 2.803 |
| ATOM | 20 | O25 | URI | 1 | −0.384 | −2.556 | −3.007 | −0.28 | −1.65 | −0.850 | 2.803 |
| ATOM | 21 | O24 | URI | 1 | −0.029 | −4.957 | −2.149 | +0.17 | −0.31 | −0.850 | 2.803 |
| ATOM | 22 | O23 | URI | 1 | 1.498 | −3.000 | −1.509 | +0.14 | −0.16 | −0.368 | 2.803 |
| ATOM | 23 | C37 | URI | 1 | 1.194 | −3.768 | −0.367 | −0.15 | +0.06 | +0.227 | 2.803 |
| ATOM | 24 | C45 | URI | 1 | 1.469 | −2.964 | 0.941 | −0.12 | +0.02 | +0.211 | 2.803 |
| ATOM | 25 | N41 | URI | 1 | 0.745 | −1.664 | 0.884 | −0.06 | +0.04 | −0.650 | 2.803 |
| ATOM | 26 | H41 | URI | 1 | 1.313 | −0.867 | 0.598 | +0.11 | −0.03 | +0.440 | 2.803 |
| ATOM | 27 | C49 | URI | 1 | −0.569 | −1.543 | 1.098 | −0.23 | −0.11 | +0.396 | 2.803 |
| ATOM | 28 | O38 | URI | 1 | −1.303 | −2.476 | 1.425 | +0.11 | +0.13 | −0.396 | 2.803 |
| ATOM | 29 | C50 | URI | 1 | −1.142 | −0.141 | 0.908 | −0.41 | +0.00 | +0.000 | 2.803 |
| ATOM | 30 | C44 | URI | 1 | 3.005 | −2.796 | 1.159 | −0.21 | +0.02 | +0.113 | 2.803 |
| ATOM | 31 | O47 | URI | 1 | 3.263 | −2.230 | 2.441 | +0.17 | −0.06 | −0.537 | 2.803 |
| ATOM | 32 | H47 | URI | 1 | 2.716 | −2.715 | 3.095 | +0.11 | +0.04 | +0.424 | 2.803 |
| ATOM | 33 | C43 | URI | 1 | 3.771 | −4.139 | 1.059 | −0.34 | +0.06 | +0.113 | 2.803 |
| ATOM | 34 | O48 | URI | 1 | 5.168 | −3.879 | 1.118 | −0.14 | −0.49 | −0.537 | 2.803 |
| ATOM | 35 | H48 | URI | 1 | 5.367 | −3.813 | 2.073 | +0.06 | +0.30 | +0.424 | 2.803 |
| ATOM | 36 | C46 | URI | 1 | 3.412 | −4.884 | −0.258 | −0.39 | +0.07 | +0.113 | 2.803 |
| ATOM | 37 | C42 | URI | 1 | 4.018 | −6.293 | −0.348 | −0.51 | +0.10 | +0.113 | 2.803 |
| ATOM | 38 | O39 | URI | 1 | 4.797 | −6.406 | −1.533 | −0.58 | −0.87 | −0.537 | 2.803 |
| ATOM | 39 | H39 | URI | 1 | 4.190 | −6.316 | −2.303 | +0.05 | +0.51 | +0.424 | 2.803 |
| ATOM | 40 | O40 | URI | 1 | 1.963 | −5.007 | −0.372 | +0.13 | −0.09 | −0.227 | 2.803 |
| ATOM | 41 | C12 | URI | 1 | −1.372 | −6.904 | −7.000 | −0.02 | +0.01 | +0.113 | 2.803 |
| ATOM | 42 | O15 | URI | 1 | −1.439 | −6.900 | −8.429 | −0.07 | −0.11 | −0.537 | 2.803 |
| ATOM | 43 | H15 | URI | 1 | −2.387 | −6.806 | −8.640 | +0.01 | +0.18 | +0.424 | 2.803 |
| ATOM | 44 | C10 | URI | 1 | −2.141 | −8.154 | −6.515 | −0.48 | +0.07 | +0.113 | 2.803 |
| ATOM | 45 | O11 | URI | 1 | −3.219 | −8.421 | −7.381 | −0.26 | −0.46 | −0.537 | 2.803 |
| ATOM | 46 | H11 | URI | 1 | −2.801 | −8.563 | −8.255 | +0.06 | +0.25 | +0.424 | 2.803 |
| TER | | | | | | | | | | | |
| ENDMDL | | | | | | | | | | | |
| MODEL | 12 | | | | | | | | | | |
| USER | Run = 12 | | | | | | | | | | |
| USER | Cluster Rank = 8 | | | | | | | | | | |
| USER | Number of conformations in this cluster = 1 | | | | | | | | | | |
| USER | | | | | | | | | | | |
| USER | RMSD from reference structure = 2.438 A | | | | | | | | | | |
| USER | | | | | | | | | | | |
| USER | Estimated Free Energy of Binding = −9.86 kcal/mol [= (1) + (3)] | | | | | | | | | | |
| USER | Estimated Inhibition Constant, Ki = +5.96e−08 [Temperature = 298.15 K] | | | | | | | | | | |
| USER | | | | | | | | | | | |
| USER | Final Docked Energy = −10.39 kcal/mol [= (1) + (2)] | | | | | | | | | | |
| USER | | | | | | | | | | | |
| USER | (1) Final Intermolecular Energy = −14.53 kcal/mol | | | | | | | | | | |
| USER | (2) Final Internal Energy of Ligand = +4.13 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 2.027481 −4.418641 −5.669839 | | | | | | | | | | |
| USER | NEWDPF quat0 0.715626 −0.186973 0.672994 −11.767848 | | | | | | | | | | |
| USER | NEWDPF ndihe 15 | | | | | | | | | | |
| USER | NEWDPF dihe0 78.04 51.41 −179.93 −35.75 120.30 −174.84 120.63 34.34 162.64 | | | | | | | | | | |
| | 142.58 1.56 179.38 −20.06 −76.82 9.58 | | | | | | | | | | |
| USER | | | | | | | | | | | |
| USER | | | | | x | y | z | vdW | Elec | q | RMS |
| ATOM | 1 | C2 | URI | 1 | −1.663 | −11.972 | −6.043 | −0.55 | +0.24 | +0.296 | 2.438 |
| ATOM | 2 | N3 | URI | 1 | −1.716 | −13.059 | −5.207 | −0.32 | −0.20 | −0.292 | 2.438 |
| ATOM | 3 | C4 | URI | 1 | −1.535 | −13.045 | −3.822 | −0.64 | +0.24 | +0.346 | 2.438 |
| ATOM | 4 | C5 | URI | 1 | −1.256 | −11.756 | −3.272 | −0.47 | +0.00 | +0.000 | 2.438 |
| ATOM | 5 | C6 | URI | 1 | −1.206 | −10.687 | −4.081 | −0.22 | −0.03 | −0.050 | 2.438 |
| ATOM | 6 | N1 | URI | 1 | −1.386 | −10.768 | −5.458 | −0.34 | +0.03 | +0.039 | 2.438 |
| ATOM | 7 | O8 | URI | 1 | −1.608 | −14.114 | −3.217 | −0.11 | −0.30 | −0.396 | 2.438 |
| ATOM | 8 | O7 | URI | 1 | −1.842 | −12.080 | −7.257 | −0.21 | −0.22 | −0.396 | 2.438 |
| ATOM | 9 | H3 | URI | 1 | −1.904 | −13.949 | −5.632 | −0.28 | +0.10 | +0.393 | 2.438 |
| ATOM | 10 | C9 | URI | 1 | −1.299 | −9.567 | −6.297 | −0.51 | +0.11 | +0.174 | 2.438 |
| ATOM | 11 | O14 | URI | 1 | 0.074 | −9.195 | −6.322 | −0.07 | −0.13 | −0.227 | 2.438 |
| ATOM | 12 | C13 | URI | 1 | 0.182 | −7.786 | −6.662 | −0.51 | +0.04 | +0.113 | 2.438 |
| ATOM | 13 | C16 | URI | 1 | 1.228 | −7.145 | −5.751 | −0.45 | +0.05 | +0.113 | 2.438 |
| ATOM | 14 | O17 | URI | 1 | 2.428 | −7.018 | −6.490 | −0.02 | −0.21 | −0.368 | 2.438 |
| ATOM | 15 | P18 | URI | 1 | 3.915 | −6.671 | −6.091 | −0.54 | +1.10 | +1.210 | 2.438 |
| ATOM | 16 | O19 | URI | 1 | 4.373 | −7.694 | −5.108 | −0.25 | −0.77 | −0.850 | 2.438 |
| ATOM | 17 | O20 | URI | 1 | 4.712 | −6.488 | −7.345 | +0.36 | −1.04 | −0.850 | 2.438 |
| ATOM | 18 | O21 | URI | 1 | 3.562 | −5.313 | −5.334 | +0.02 | −0.49 | −0.510 | 2.438 |
| ATOM | 19 | P22 | URI | 1 | 4.289 | −4.486 | −4.176 | −0.54 | +1.46 | +1.210 | 2.438 |
| ATOM | 20 | O25 | URI | 1 | 4.841 | −5.319 | −3.078 | −0.18 | −1.39 | −0.850 | 2.438 |
| ATOM | 21 | O24 | URI | 1 | 5.185 | −3.612 | −4.974 | −0.19 | −1.55 | −0.850 | 2.438 |
| ATOM | 22 | O23 | URI | 1 | 3.107 | −3.640 | −3.472 | +0.06 | −0.32 | −0.368 | 2.438 |
| ATOM | 23 | C37 | URI | 1 | 2.150 | −4.644 | −3.221 | −0.29 | +0.15 | +0.227 | 2.438 |
| ATOM | 24 | C45 | URI | 1 | 0.757 | −4.252 | −3.806 | −0.25 | +0.17 | +0.211 | 2.438 |
| ATOM | 25 | N41 | URI | 1 | 0.904 | −3.913 | −5.248 | −0.18 | −0.49 | −0.650 | 2.438 |
| ATOM | 26 | H41 | URI | 1 | 0.699 | −4.676 | −5.892 | −0.12 | +0.03 | +0.440 | 2.438 |
| ATOM | 27 | C49 | URI | 1 | 1.191 | −2.684 | −5.691 | −0.36 | +0.54 | +0.396 | 2.438 |
| ATOM | 28 | O38 | URI | 1 | 1.426 | −1.724 | −4.955 | −0.49 | −0.74 | +0.396 | 2.438 |
| ATOM | 29 | C50 | URI | 1 | 1.212 | −2.518 | −7.207 | −0.56 | +0.00 | +0.000 | 2.438 |
| ATOM | 30 | C44 | URI | 1 | 0.126 | −3.095 | −2.970 | −0.12 | +0.15 | +0.113 | 2.438 |
| ATOM | 31 | O47 | URI | 1 | −1.218 | −2.864 | −3.382 | −0.26 | −1.00 | −0.537 | 2.438 |
| ATOM | 32 | H47 | URI | 1 | −1.731 | −3.677 | −3.188 | +0.07 | +0.23 | +0.424 | 2.438 |
| ATOM | 33 | C43 | URI | 1 | 0.123 | −3.393 | −1.449 | −0.17 | +0.04 | +0.113 | 2.438 |
| ATOM | 34 | O48 | URI | 1 | −0.326 | −2.236 | −0.754 | +0.14 | −0.07 | −0.537 | 2.438 |
| ATOM | 35 | H48 | URI | 1 | −1.287 | −2.375 | −0.637 | +0.08 | −0.04 | +0.424 | 2.438 |
| ATOM | 36 | C46 | URI | 1 | 1.540 | −3.804 | −0.962 | −0.19 | +0.04 | +0.113 | 2.438 |
| ATOM | 37 | C42 | URI | 1 | 1.574 | −4.262 | 0.505 | −0.15 | +0.03 | +0.113 | 2.438 |
| ATOM | 38 | O39 | URI | 1 | 2.391 | −3.379 | 1.265 | +0.16 | −0.11 | −0.537 | 2.438 |
| ATOM | 39 | H39 | URI | 1 | 2.734 | −2.685 | 0.656 | +0.10 | +0.09 | +0.424 | 2.438 |
| ATOM | 40 | O40 | URI | 1 | 2.045 | −4.896 | −1.788 | +0.12 | −0.12 | −0.227 | 2.438 |
| ATOM | 41 | C12 | URI | 1 | −1.219 | −7.210 | −6.463 | −0.40 | +0.02 | +0.113 | 2.438 |
| ATOM | 42 | O15 | URI | 1 | −1.864 | −7.061 | −7.733 | −0.15 | −0.15 | −0.537 | 2.438 |
| ATOM | 43 | H15 | URI | 1 | −2.016 | −6.102 | −7.831 | −0.21 | −0.10 | +0.424 | 2.438 |
| ATOM | 44 | C10 | URI | 1 | −1.972 | −8.326 | −5.704 | −0.39 | +0.07 | +0.113 | 2.438 |
| ATOM | 45 | O11 | URI | 1 | −3.340 | −8.297 | −6.038 | −0.23 | −0.64 | −0.537 | 2.438 |
| ATOM | 46 | H11 | URI | 1 | −3.611 | −9.239 | −6.037 | +0.08 | +0.41 | +0.424 | 2.438 |
| TER | | | | | | | | | | | |
| ENDMDL | | | | | | | | | | | |
| MODEL | 39 | | | | | | | | | | |
| USER | Run = 39 | | | | | | | | | | |
| USER | Cluster Rank = 9 | | | | | | | | | | |
| USER | Number of conformations in this cluster = 1 | | | | | | | | | | |
| USER | | | | | | | | | | | |
| USER | RMSD from reference structure = 5.527 A | | | | | | | | | | |
| USER | | | | | | | | | | | |
| USER | Estimated Free Energy of Binding = −6.98 kcal/mol [= (1) + (3)] | | | | | | | | | | |
| USER | Estimated Inhibition Constant, Ki = +7.71e−06 [Temperature = 298.15 K] | | | | | | | | | | |
| USER | | | | | | | | | | | |
| USER | Final Docked Energy = −9.79 kcal/mol [= (1) + (2)] | | | | | | | | | | |
| USER | | | | | | | | | | | |
| USER | (1) Final Intermolecular Energy = −11.64 kcal/mol | | | | | | | | | | |
| USER | (2) Final Internal Energy of Ligand = +1.86 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −2.581537 −7.991511 −5.345211 | | | | | | | | | | |
| USER | NEWDPF quat0 −0.672399 0.359027 −0.647286 107.092580 | | | | | | | | | | |
| USER | NEWDPF ndihe 15 | | | | | | | | | | |
| USER | NEWDPF dihe0 179.88 −176.50 135.25 132.56 128.87 10.03 −32.61 −5.92 −41.19 | | | | | | | | | | |
| | −23.36 118.22 −42.55 −86.27 −89.07 32.56 | | | | | | | | | | |
| USER | | | | | | | | | | | |
| USER | | | | | x | y | z | vdW | Elec | q | RMS |
| ATOM | 1 | C2 | URI | 1 | −7.212 | −5.146 | 1.079 | −0.65 | −0.14 | +0.296 | 5.527 |
| ATOM | 2 | N3 | URI | 1 | −6.951 | −4.836 | 2.390 | −0.41 | +0.13 | −0.292 | 5.527 |
| ATOM | 3 | C4 | URI | 1 | −5.712 | −4.473 | 2.921 | −0.57 | −0.12 | +0.346 | 5.527 |
| ATOM | 4 | C5 | URI | 1 | −4.643 | −4.448 | 1.973 | −0.35 | +0.00 | +0.000 | 5.527 |
| ATOM | 5 | C6 | URI | 1 | −4.890 | −4.745 | 0.689 | −0.37 | +0.02 | −0.050 | 5.527 |
| ATOM | 6 | N1 | URI | 1 | −6.148 | −5.111 | 0.222 | −0.30 | −0.01 | +0.039 | 5.527 |
| ATOM | 7 | O8 | URI | 1 | −5.645 | −4.232 | 4.126 | −0.19 | −0.01 | −0.396 | 5.527 |
| ATOM | 8 | O7 | URI | 1 | −8.343 | −5.452 | 0.699 | −0.19 | +0.26 | −0.396 | 5.527 |
| ATOM | 9 | H3 | URI | 1 | −7.725 | −4.875 | 3.028 | +0.08 | −0.24 | +0.393 | 5.527 |
| ATOM | 10 | C9 | URI | 1 | −6.354 | −5.446 | −1.191 | −0.54 | −0.04 | +0.174 | 5.527 |
| ATOM | 11 | O14 | URI | 1 | −6.227 | −6.860 | −1.290 | +0.07 | −0.02 | −0.227 | 5.527 |
| ATOM | 12 | C13 | URI | 1 | −5.895 | −7.217 | −2.659 | −0.42 | +0.05 | +0.113 | 5.527 |
| ATOM | 13 | C16 | URI | 1 | −4.806 | −8.289 | −2.632 | −0.27 | +0.06 | +0.113 | 5.527 |
| ATOM | 14 | O17 | URI | 1 | −5.442 | −9.552 | −2.578 | +0.11 | −0.28 | −0.368 | 5.527 |
| ATOM | 15 | P18 | URI | 1 | −5.343 | −10.854 | −3.464 | −0.14 | +1.22 | +1.210 | 5.527 |
| ATOM | 16 | O19 | URI | 1 | −6.540 | −11.694 | −3.176 | −0.20 | −1.05 | −0.850 | 5.527 |
| ATOM | 17 | O20 | URI | 1 | −5.091 | −10.444 | −4.883 | −0.07 | −1.05 | −0.850 | 5.527 |
| ATOM | 18 | O21 | URI | 1 | −4.068 | −11.454 | −2.718 | +0.12 | −0.31 | −0.510 | 5.527 |
| ATOM | 19 | P22 | URI | 1 | −2.493 | −11.277 | −2.930 | −0.34 | +0.70 | +1.210 | 5.527 |
| ATOM | 20 | O25 | URI | 1 | −1.683 | −11.456 | −1.699 | +0.01 | −0.43 | −0.850 | 5.527 |
| ATOM | 21 | O24 | URI | 1 | −2.268 | −12.153 | −4.108 | −0.07 | −0.69 | −0.850 | 5.527 |
| ATOM | 22 | O23 | URI | 1 | −2.303 | −9.718 | −3.305 | +0.11 | −0.19 | −0.368 | 5.527 |
| ATOM | 23 | C37 | URI | 1 | −1.119 | −9.752 | −4.071 | −0.08 | +0.13 | +0.227 | 5.527 |
| ATOM | 24 | C45 | URI | 1 | −0.548 | −8.317 | −4.286 | −0.25 | +0.09 | +0.211 | 5.527 |
| ATOM | 25 | N41 | URI | 1 | −0.386 | −7.646 | −2.967 | −0.18 | −0.22 | −0.650 | 5.527 |
| ATOM | 26 | H41 | URI | 1 | −1.239 | −7.579 | −2.411 | +0.11 | +0.13 | +0.440 | 5.527 |
| ATOM | 27 | C49 | URI | 1 | 0.793 | −7.237 | −2.487 | −0.31 | +0.13 | +0.396 | 5.527 |
| ATOM | 28 | O38 | URI | 1 | 1.854 | −7.314 | −3.108 | +0.06 | −0.17 | −0.396 | 5.527 |
| ATOM | 29 | C50 | URI | 1 | 0.765 | −6.651 | −1.078 | −0.28 | +0.00 | +0.000 | 5.527 |
| ATOM | 30 | C44 | URI | 1 | −1.455 | −7.515 | −5.270 | −0.35 | +0.04 | +0.113 | 5.527 |
| ATOM | 31 | O47 | URI | 1 | −0.838 | −6.277 | −5.614 | +0.15 | +0.03 | −0.537 | 5.527 |
| ATOM | 32 | H47 | URI | 1 | −1.306 | −5.921 | −6.398 | −0.15 | −0.35 | +0.424 | 5.527 |
| ATOM | 33 | C43 | URI | 1 | −1.746 | −8.292 | −6.579 | −0.48 | +0.06 | +0.113 | 5.527 |
| ATOM | 34 | O48 | URI | 1 | −2.694 | −7.561 | −7.347 | −0.34 | −0.41 | −0.537 | 5.527 |
| ATOM | 35 | H48 | URI | 1 | −3.348 | −8.229 | −7.634 | +0.06 | +0.37 | +0.424 | 5.527 |
| ATOM | 36 | C46 | URI | 1 | −2.282 | −9.718 | −6.268 | −0.50 | +0.08 | +0.113 | 5.527 |
| ATOM | 37 | C42 | URI | 1 | −2.444 | −10.596 | −7.519 | −0.65 | +0.07 | +0.113 | 5.527 |
| ATOM | 38 | O39 | URI | 1 | −1.164 | −10.898 | −8.064 | −0.23 | −0.39 | −0.537 | 5.527 |
| ATOM | 39 | H39 | URI | 1 | −0.636 | −10.067 | −8.070 | +0.06 | +0.32 | +0.424 | 5.527 |
| ATOM | 40 | O40 | URI | 1 | −1.368 | −10.397 | −5.356 | −0.02 | −0.16 | −0.227 | 5.527 |
| ATOM | 41 | C12 | URI | 1 | −5.439 | −5.917 | −3.320 | −0.52 | +0.02 | +0.113 | 5.527 |
| ATOM | 42 | O15 | URI | 1 | −6.495 | −5.391 | −4.131 | +0.48 | +0.10 | −0.537 | 5.527 |
| ATOM | 43 | H15 | URI | 1 | −7.278 | −5.371 | −3.548 | −0.21 | −0.17 | +0.424 | 5.527 |
| ATOM | 44 | C10 | URI | 1 | −5.256 | −4.945 | −2.132 | −0.57 | −0.02 | +0.113 | 5.527 |
| ATOM | 45 | O11 | URI | 1 | −5.523 | −3.625 | −2.545 | −0.09 | +0.21 | −0.537 | 5.527 |
| ATOM | 46 | H11 | URI | 1 | −5.813 | −3.167 | −1.729 | −0.28 | −0.27 | +0.424 | 5.527 |
| TER | | | | | | | | | | | |
| ENDMDL | | | | | | | | | | | |
| MODEL | 28 | | | | | | | | | | |
| USER | Run = 28 | | | | | | | | | | |
| USER | Cluster Rank = 10 | | | | | | | | | | |
| USER | Number of conformations in this cluster = 1 | | | | | | | | | | |
| USER | | | | | | | | | | | |
| USER | RMSD from reference structure = 4.357 A | | | | | | | | | | |
| USER | | | | | | | | | | | |
| USER | Estimated Free Energy of Binding = −6.17 kcal/mol [= (1) + (3)] | | | | | | | | | | |
| USER | Estimated Inhibition Constant, Ki = +3.00e−05 [Temperature = 298.15 K] | | | | | | | | | | |
| USER | | | | | | | | | | | |
| USER | Final Docked Energy = −9.65 kcal/mol [= (1) + (2)] | | | | | | | | | | |
| USER | | | | | | | | | | | |
| USER | (1) Final intermolecular Energy = −10.84 kcal/mol | | | | | | | | | | |
| USER | (2) Final Internal Energy of Ligand = +1.19 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −3.037622 −8.510808 −5.038720 | | | | | | | | | | |
| USER | NEWDPF quat0 0.695391 −0.504554 −0.511720 −156.491601 | | | | | | | | | | |
| USER | NEWDPF ndihe 15 | | | | | | | | | | |
| USER | NEWDPF dihe0 −149.28 180.00 −16.61 29.62 −70.80 118.86 −46.37 51.32 1.56 | | | | | | | | | | |
| | 113.23 121.73 −170.31 −41.23 −90.40 −2.06 | | | | | | | | | | |
| USER | | | | | | | | | | | |
| USER | | | | | x | y | z | vdW | Elec | q | RMS |
| ATOM | 1 | C2 | URI | 1 | 4.594 | −5.195 | −3.786 | −0.60 | +0.39 | +0.296 | 4.357 |
| ATOM | 2 | N3 | URI | 1 | 5.322 | −4.201 | −4.390 | −0.40 | −0.48 | −0.292 | 4.357 |
| ATOM | 3 | C4 | URI | 1 | 4.808 | −3.129 | −5.122 | −0.66 | +0.57 | +0.346 | 4.357 |
| ATOM | 4 | C5 | URI | 1 | 3.383 | −3.117 | −5.236 | −0.51 | +0.00 | +0.000 | 4.357 |
| ATOM | 5 | C6 | URI | 1 | 2.663 | −4.081 | −4.644 | −0.36 | −0.04 | −0.050 | 4.357 |
| ATOM | 6 | N1 | URI | 1 | 3.237 | −5.131 | −3.936 | −0.25 | +0.03 | +0.039 | 4.357 |
| ATOM | 7 | O8 | URI | 1 | 5.602 | −2.324 | −5.608 | −0.10 | −0.89 | −0.396 | 4.357 |
| ATOM | 8 | O7 | URI | 1 | 5.137 | −6.102 | −3.154 | −0.39 | −0.69 | −0.396 | 4.357 |
| ATOM | 9 | H3 | URI | 1 | 6.321 | −4.251 | −4.297 | +0.01 | +0.77 | +0.393 | 4.357 |
| ATOM | 10 | C9 | URI | 1 | 2.399 | −6.171 | −3.327 | −0.39 | +0.10 | +0.174 | 4.357 |
| ATOM | 11 | O14 | URI | 1 | 1.670 | −6.771 | −4.391 | +0.04 | −0.11 | −0.227 | 4.357 |
| ATOM | 12 | C13 | URI | 1 | 0.479 | −7.408 | −3.855 | −0.30 | +0.04 | +0.113 | 4.357 |
| ATOM | 13 | C16 | URI | 1 | −0.699 | −7.092 | −4.778 | −0.30 | +0.03 | +0.113 | 4.357 |
| ATOM | 14 | O17 | URI | 1 | −0.731 | −8.078 | −5.793 | +0.05 | −0.15 | −0.368 | 4.357 |
| ATOM | 15 | P18 | URI | 1 | −1.774 | −8.439 | −6.921 | −0.50 | +0.66 | +1.210 | 4.357 |
| ATOM | 16 | O19 | URI | 1 | −1.761 | −7.336 | −7.923 | −0.12 | −0.29 | −0.850 | 4.357 |
| ATOM | 17 | O20 | URI | 1 | −1.495 | −9.832 | −7.396 | −0.05 | −0.53 | −0.850 | 4.357 |
| ATOM | 18 | O21 | URI | 1 | −3.062 | −8.309 | −5.990 | −0.27 | −0.52 | −0.510 | 4.357 |
| ATOM | 19 | P22 | URI | 1 | −3.795 | −9.319 | −4.990 | −0.42 | +1.11 | +1.210 | 4.357 |
| ATOM | 20 | O25 | URI | 1 | −4.521 | −10.428 | −5.660 | −0.16 | −1.03 | −0.850 | 4.357 |
| ATOM | 21 | O24 | URI | 1 | −2.698 | −9.613 | −4.034 | +0.10 | −0.53 | −0.850 | 4.357 |
| ATOM | 22 | O23 | URI | 1 | −4.962 | −8.441 | −4.303 | −0.20 | −0.48 | −0.368 | 4.357 |
| ATOM | 23 | C37 | URI | 1 | −4.489 | −8.324 | −2.980 | −0.28 | +0.14 | +0.227 | 4.357 |
| ATOM | 24 | C45 | URI | 1 | −5.365 | −9.157 | −1.994 | −0.21 | +0.11 | +0.211 | 4.357 |
| ATOM | 25 | N41 | URI | 1 | −5.452 | −10.563 | −2.477 | −0.16 | −0.49 | −0.650 | 4.357 |
| ATOM | 26 | H41 | URI | 1 | −6.019 | −10.697 | −3.314 | +0.08 | +0.60 | +0.440 | 4.357 |
| ATOM | 27 | C49 | URI | 1 | −4.773 | −11.575 | −1.925 | −0.18 | +0.21 | +0.396 | 4.357 |
| ATOM | 28 | O38 | URI | 1 | −4.059 | −11.463 | −0.928 | +0.19 | −0.15 | −0.396 | 4.357 |
| ATOM | 29 | C50 | URI | 1 | −4.926 | −12.926 | −2.617 | −0.24 | +0.00 | +0.000 | 4.357 |
| ATOM | 30 | C44 | URI | 1 | −6.764 | −8.488 | −1.816 | −0.22 | +0.05 | +0.113 | 4.357 |
| ATOM | 31 | O47 | URI | 1 | −7.493 | −9.137 | −0.778 | +0.10 | −0.16 | −0.537 | 4.357 |
| ATOM | 32 | H47 | URI | 1 | −8.433 | −8.873 | −0.867 | +0.09 | +0.10 | +0.424 | 4.357 |
| ATOM | 33 | C43 | URI | 1 | −6.661 | −6.981 | −1.467 | −0.41 | +0.01 | +0.113 | 4.357 |
| ATOM | 34 | O48 | URI | 1 | −7.968 | −6.418 | −1.471 | −0.08 | +0.16 | −0.537 | 4.357 |
| ATOM | 35 | H48 | URI | 1 | −7.866 | −5.560 | −1.928 | −0.23 | −0.23 | +0.424 | 4.357 |
| ATOM | 36 | C46 | URI | 1 | −5.747 | −6.239 | −2.481 | −0.51 | +0.02 | +0.113 | 4.357 |
| ATOM | 37 | C42 | URI | 1 | −5.480 | −4.773 | −2.105 | −0.62 | −0.02 | +0.113 | 4.357 |
| ATOM | 38 | O39 | URI | 1 | −4.577 | −4.194 | −3.040 | −0.14 | +0.07 | −0.537 | 4.357 |
| ATOM | 39 | H39 | URI | 1 | −4.630 | −3.216 | −2.945 | −0.23 | −0.18 | +0.424 | 4.357 |
| ATOM | 40 | O40 | URI | 1 | −4.462 | −6.923 | −2.575 | +0.09 | −0.07 | −0.227 | 4.357 |
| ATOM | 41 | C12 | URI | 1 | 0.307 | −6.835 | −2.449 | −0.27 | +0.04 | +0.113 | 4.357 |
| ATOM | 42 | O15 | URI | 1 | 0.769 | −7.784 | −1.482 | −0.01 | −0.13 | −0.537 | 4.357 |
| ATOM | 43 | H15 | URI | 1 | 0.312 | −8.617 | −1.704 | +0.06 | +0.11 | +0.424 | 4.357 |
| ATOM | 44 | C10 | URI | 1 | 1.289 | −5.642 | −2.416 | −0.25 | +0.05 | +0.113 | 4.357 |
| ATOM | 45 | O11 | URI | 1 | 1.772 | −5.452 | −1.106 | +0.12 | −0.22 | −0.537 | 4.357 |
| ATOM | 46 | H11 | URI | 1 | 0.972 | −5.500 | −0.542 | +0.11 | +0.12 | +0.424 | 4.357 |
| TER | | | | | | | | | | | |
| ENDMDL | | | | | | | | | | | |
| MODEL | 44 | | | | | | | | | | |
| USER | Run = 44 | | | | | | | | | | |
| USER | Cluster Rank = 11 | | | | | | | | | | |
| USER | Number of conformations in this cluster = 1 | | | | | | | | | | |
| USER | | | | | | | | | | | |
| USER | RMSD from reference structure = 3.877 A | | | | | | | | | | |
| USER | | | | | | | | | | | |
| USER | Estimated Free Energy of Binding = −6.56 kcal/mol [= (1) + (3)] | | | | | | | | | | |
| USER | Estimated Inhibition Constant, Ki = +1.55e−05 [Temperature = 298.15 K] | | | | | | | | | | |
| USER | | | | | | | | | | | |
| USER | Final Docked Energy = −9.40 kcal/mol [= (1) + (2)] | | | | | | | | | | |
| USER | | | | | | | | | | | |
| USER | (1) Final Intermolecular Energy = −11.23 kcal/mol | | | | | | | | | | |
| USER | (2) Final Internal Energy of Ligand = +1.83 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.067488 −10.479574 −1.686235 | | | | | | | | | | |
| USER | NEWDPF quat0 −0.579835 −0.808480 0.100755 −60.250381 | | | | | | | | | | |
| USER | NEWDPF ndihe 15 | | | | | | | | | | |
| USER | NEWDPF dihe0 28.25 −120.32 21.68 7.87 163.91 −185.40 −47.71 47.91 13.93 | | | | | | | | | | |
| | −177.91 55.17 −97.61 −132.85 −113.21 34.54 | | | | | | | | | | |
| USER | | | | | | | | | | | |
| USER | | | | | x | y | z | vdW | Elec | q | RMS |
| ATOM | 1 | C2 | URI | 1 | −4.286 | −16.450 | −4.239 | −0.35 | +0.14 | +0.296 | 3.877 |
| ATOM | 2 | N3 | URI | 1 | −4.173 | −17.789 | −4.512 | −0.15 | −0.08 | −0.292 | 3.877 |
| ATOM | 3 | C4 | URI | 1 | −3.207 | −18.658 | −4.000 | −0.28 | +0.13 | +0.346 | 3.877 |
| ATOM | 4 | C5 | URI | 1 | −2.261 | −18.044 | −3.123 | −0.41 | +0.00 | +0.000 | 3.877 |
| ATOM | 5 | C6 | URI | 1 | −2.366 | −16.736 | −2.846 | −0.43 | −0.03 | −0.050 | 3.877 |
| ATOM | 6 | N1 | URI | 1 | −3.347 | −15.920 | −3.398 | −0.23 | +0.02 | +0.039 | 3.877 |
| ATOM | 7 | O8 | URI | 1 | −3.241 | −19.837 | −4.353 | +0.13 | −0.13 | −0.396 | 3.877 |
| ATOM | 8 | O7 | URI | 1 | −5.177 | −15.760 | −4.735 | −0.12 | −0.19 | −0.396 | 3.877 |
| ATOM | 9 | H3 | URI | 1 | −4.850 | −18.182 | −5.141 | +0.04 | +0.03 | +0.393 | 3.877 |
| ATOM | 10 | C9 | URI | 1 | −3.400 | −14.489 | −3.075 | −0.32 | +0.12 | +0.174 | 3.877 |
| ATOM | 11 | O14 | URI | 1 | −2.717 | −13.816 | −4.126 | +0.06 | −0.21 | −0.227 | 3.877 |
| ATOM | 12 | C13 | URI | 1 | −2.269 | −12.518 | −3.652 | −0.46 | +0.08 | +0.113 | 3.877 |
| ATOM | 13 | C16 | URI | 1 | −0.841 | −12.285 | −4.147 | −0.50 | +0.08 | +0.113 | 3.877 |
| ATOM | 14 | O17 | URI | 1 | −0.879 | −12.210 | −5.559 | −0.28 | −0.32 | −0.368 | 3.877 |
| ATOM | 15 | P18 | URI | 1 | −1.282 | −11.071 | −6.573 | −0.69 | +0.95 | +1.210 | 3.877 |
| ATOM | 16 | O19 | URI | 1 | −0.345 | −11.138 | −7.731 | −0.38 | −0.90 | −0.850 | 3.877 |
| ATOM | 17 | O20 | URI | 1 | −2.751 | −11.175 | −6.844 | −0.22 | −0.79 | −0.850 | 3.877 |
| ATOM | 18 | O21 | URI | 1 | −0.876 | −9.860 | −5.618 | −0.01 | −0.32 | −0.510 | 3.877 |
| ATOM | 19 | P22 | URI | 1 | −1.404 | −8.358 | −5.472 | −0.37 | +0.59 | +1.210 | 3.877 |
| ATOM | 20 | O25 | URI | 1 | −2.456 | −7.978 | −6.448 | −0.26 | −0.64 | −0.850 | 3.877 |
| ATOM | 21 | O24 | URI | 1 | −1.679 | −8.298 | −4.014 | +0.11 | −0.38 | −0.850 | 3.877 |
| ATOM | 22 | O23 | URI | 1 | −0.140 | −7.438 | −5.876 | +0.05 | −0.11 | −0.368 | 3.877 |
| ATOM | 23 | C37 | URI | 1 | 0.384 | −7.104 | −4.611 | −0.33 | +0.08 | +0.227 | 3.877 |
| ATOM | 24 | C45 | URI | 1 | 0.232 | −5.577 | −4.325 | −0.26 | +0.07 | +0.211 | 3.877 |
| ATOM | 25 | N41 | URI | 1 | −1.191 | −5.180 | −4.507 | −0.17 | −0.07 | −0.650 | 3.877 |
| ATOM | 26 | H41 | URI | 1 | −1.510 | −5.153 | −5.475 | −0.15 | −0.30 | +0.440 | 3.877 |
| ATOM | 27 | C49 | URI | 1 | −2.030 | −4.960 | −3.490 | −0.34 | +0.09 | +0.396 | 3.877 |
| ATOM | 28 | O38 | URI | 1 | −1.701 | −5.000 | −2.304 | +0.14 | −0.08 | −0.396 | 3.877 |
| ATOM | 29 | C50 | URI | 1 | −3.472 | −4.648 | −3.880 | −0.53 | +0.00 | +0.000 | 3.877 |
| ATOM | 30 | C44 | URI | 1 | 1.212 | −4.759 | −5.222 | −0.30 | +0.05 | +0.113 | 3.877 |
| ATOM | 31 | O47 | URI | 1 | 1.211 | −3.389 | −4.832 | −0.19 | −0.65 | −0.537 | 3.877 |
| ATOM | 32 | H47 | URI | 1 | 0.764 | −3.329 | −3.962 | +0.09 | +0.64 | +0.424 | 3.877 |
| ATOM | 33 | C43 | URI | 1 | 2.668 | −5.285 | −5.145 | −0.38 | +0.08 | +0.113 | 3.877 |
| ATOM | 34 | O48 | URI | 1 | 3.461 | −4.586 | −6.099 | +0.02 | −0.57 | −0.537 | 3.877 |
| ATOM | 35 | H48 | URI | 1 | 3.238 | −5.001 | −6.956 | +0.09 | +0.39 | +0.424 | 3.877 |
| ATOM | 36 | C46 | URI | 1 | 2.720 | −6.815 | −5.412 | −0.53 | +0.07 | +0.113 | 3.877 |
| ATOM | 37 | C42 | URI | 1 | 4.114 | −7.426 | −5.199 | −0.74 | +0.10 | +0.113 | 3.877 |
| ATOM | 38 | O39 | URI | 1 | 4.944 | −7.129 | −6.316 | −0.15 | −0.59 | −0.537 | 3.877 |
| ATOM | 39 | H39 | URI | 1 | 5.673 | −7.791 | −6.334 | +0.01 | +0.43 | +0.424 | 3.877 |
| ATOM | 40 | O40 | URI | 1 | 1.786 | −7.499 | −4.524 | −0.01 | −0.11 | −0.227 | 3.877 |
| ATOM | 41 | C12 | URI | 1 | −2.371 | −12.583 | −2.128 | −0.38 | +0.06 | +0.113 | 3.877 |
| ATOM | 42 | O15 | URI | 1 | −3.548 | −11.894 | −1.694 | +0.16 | −0.25 | −0.537 | 3.877 |
| ATOM | 43 | H15 | URI | 1 | −3.347 | −10.947 | −1.809 | +0.11 | +0.19 | +0.424 | 3.877 |
| ATOM | 44 | C10 | URI | 1 | −2.594 | −14.085 | −1.839 | −0.25 | +0.07 | +0.113 | 3.877 |
| ATOM | 45 | O11 | URI | 1 | −3.367 | −14.240 | −0.671 | +0.13 | −0.23 | −0.537 | 3.877 |
| ATOM | 46 | H11 | URI | 1 | −3.342 | −15.202 | −0.488 | +0.10 | +0.17 | +0.424 | 3.877 |
| TER | | | | | | | | | | | |
| ENDMDL | | | | | | | | | | | |
| MODEL | 36 | | | | | | | | | | |
| USER | Run = 36 | | | | | | | | | | |
| USER | Cluster Rank = 12 | | | | | | | | | | |
| USER | Number of conformations in this cluster = 1 | | | | | | | | | | |
| USER | | | | | | | | | | | |
| USER | RMSD from reference structure = 3.256 A | | | | | | | | | | |
| USER | | | | | | | | | | | |
| USER | Estimated Free Energy of Binding = −6.70 kcal/mol [= (1) + (3)] | | | | | | | | | | |
| USER | Estimated Inhibition Constant, Ki = +1.22e−05 [Temperature = 298.15 K] | | | | | | | | | | |
| USER | | | | | | | | | | | |
| USER | Final Docked Energy = −9.27 kcal/mol [= (1) + (2)] | | | | | | | | | | |
| USER | | | | | | | | | | | |
| USER | (1) Final Intermolecular Energy = −11.37 kcal/mol | | | | | | | | | | |
| USER | (2) Final Internal Energy of Ligand = +2.10 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 2.641363 −5.603286 −2.454735 | | | | | | | | | | |
| USER | NEWDPF quat0 −0.755154 −0.647598 −0.101781 −88.633354 | | | | | | | | | | |
| USER | NEWDPF ndihe 15 | | | | | | | | | | |
| USER | NEWDPF dihe0 −128.58 −166.55 −120.86 −6.96 −121.92 −15.02 −32.21 36.24 | | | | | | | | | | |
| | 55.26 −128.98 111.30 −170.04 −110.40 −74.38 17.54 | | | | | | | | | | |
| USER | | | | | | | | | | | |
| USER | | | | | x | y | z | vdW | Elec | q | RMS |
| ATOM | 1 | C2 | URI | 1 | −2.981 | −9.372 | −7.455 | −0.66 | +0.20 | +0.296 | 3.256 |
| ATOM | 2 | N3 | URI | 1 | −2.903 | −10.307 | −8.456 | −0.34 | −0.15 | −0.292 | 3.256 |
| ATOM | 3 | C4 | URI | 1 | −1.889 | −11.252 | −8.626 | −0.79 | +0.19 | +0.346 | 3.256 |
| ATOM | 4 | C5 | URI | 1 | −0.849 | −11.190 | −7.648 | −0.74 | +0.00 | +0.000 | 3.256 |
| ATOM | 5 | C6 | URI | 1 | −0.919 | −10.282 | −6.663 | −0.64 | −0.04 | −0.050 | 3.256 |
| ATOM | 6 | N1 | URI | 1 | −1.953 | −9.358 | −6.554 | −0.28 | −0.02 | +0.039 | 3.256 |
| ATOM | 7 | O8 | URI | 1 | −1.966 | −12.019 | −9.585 | −0.37 | −0.14 | −0.396 | 3.256 |
| ATOM | 8 | O7 | URI | 1 | −3.920 | −8.578 | −7.382 | −0.23 | −0.40 | −0.396 | 3.256 |
| ATOM | 9 | H3 | URI | 1 | −3.647 | −10.312 | −9.130 | +0.03 | +0.15 | +0.393 | 3.256 |
| ATOM | 10 | C9 | URI | 1 | −1.966 | −8.373 | −5.467 | −0.37 | −0.10 | +0.174 | 3.256 |
| ATOM | 11 | O14 | URI | 1 | −1.063 | −7.342 | −5.851 | +0.04 | −0.07 | −0.227 | 3.256 |
| ATOM | 12 | C13 | URI | 1 | −0.619 | −6.628 | −4.666 | −0.28 | +0.03 | +0.113 | 3.256 |
| ATOM | 13 | C16 | URI | 1 | 0.886 | −6.385 | −4.779 | −0.32 | +0.04 | +0.113 | 3.256 |
| ATOM | 14 | O17 | URI | 1 | 1.081 | −5.029 | −5.131 | +0.08 | −0.16 | −0.368 | 3.256 |
| ATOM | 15 | P18 | URI | 1 | 1.495 | −3.745 | −4.312 | −0.29 | +1.14 | +1.210 | 3.256 |
| ATOM | 16 | O19 | URI | 1 | 2.924 | −3.897 | −3.919 | +0.07 | −0.74 | −0.850 | 3.256 |
| ATOM | 17 | O20 | URI | 1 | 1.101 | −2.533 | −5.098 | −0.34 | −1.65 | −0.850 | 3.256 |
| ATOM | 18 | O21 | URI | 1 | 0.554 | −4.050 | −3.060 | +0.08 | −0.38 | −0.51 | 3.256 |
| ATOM | 19 | P22 | URI | 1 | −0.917 | −3.572 | −2.657 | −0.27 | +0.84 | +1.210 | 3.256 |
| ATOM | 20 | O25 | URI | 1 | −1.913 | −3.654 | −3.755 | −0.26 | −0.47 | −0.850 | 3.256 |
| ATOM | 21 | O24 | URI | 1 | −1.125 | −4.335 | −1.400 | +0.16 | −0.15 | −0.850 | 3.256 |
| ATOM | 22 | O23 | URI | 1 | −0.771 | −1.989 | −2.373 | −0.24 | −0.35 | −0.368 | 3.256 |
| ATOM | 23 | C37 | URI | 1 | 0.228 | −1.970 | −1.378 | −0.24 | +0.09 | +0.227 | 3.256 |
| ATOM | 24 | C45 | URI | 1 | −0.257 | −1.202 | −0.110 | −0.25 | −0.01 | +0.211 | 3.256 |
| ATOM | 25 | N41 | URI | 1 | −1.550 | −1.777 | 0.353 | −0.22 | +0.22 | −0.650 | 3.256 |
| ATOM | 26 | H41 | URI | 1 | −2.296 | −1.774 | −0.344 | −0.19 | −0.25 | +0.440 | 3.256 |
| ATOM | 27 | C49 | URI | 1 | −1.718 | −2.343 | 1.552 | −0.33 | −0.17 | +0.396 | 3.256 |
| ATOM | 28 | O38 | URI | 1 | −0.845 | −2.391 | 2.420 | +0.13 | +0.13 | −0.396 | 3.256 |
| ATOM | 29 | C50 | URI | 1 | −3.093 | −2.954 | 1.807 | −0.17 | −0.00 | +0.000 | 3.256 |
| ATOM | 30 | C44 | URI | 1 | −0.342 | 0.329 | −0.404 | −0.37 | −0.02 | −0.113 | 3.256 |
| ATOM | 31 | O47 | URI | 1 | −0.601 | 1.049 | 0.798 | +0.05 | +0.28 | −0.537 | 3.256 |
| ATOM | 32 | H47 | URI | 1 | −1.197 | 1.795 | 0.575 | +0.09 | −0.25 | +0.424 | 3.256 |
| ATOM | 33 | C43 | URI | 1 | 0.959 | 0.886 | −1.033 | −0.40 | +0.00 | +0.113 | 3.256 |
| ATOM | 34 | O48 | URI | 1 | 0.746 | 2.243 | −1.406 | −0.07 | +0.09 | −0.537 | 3.256 |
| ATOM | 35 | H48 | URI | 1 | 0.961 | 2.274 | −2.359 | −0.25 | −0.09 | +0.424 | 3.256 |
| ATOM | 36 | C46 | URI | 1 | 1.383 | 0.044 | −2.268 | −0.36 | +0.04 | +0.113 | 3.256 |
| ATOM | 37 | C42 | URI | 1 | 2.747 | 0.449 | −2.847 | −0.44 | +0.02 | +0.113 | 3.256 |
| ATOM | 38 | O39 | URI | 1 | 3.671 | 0.671 | −1.787 | −0.02 | +0.05 | −0.537 | 3.256 |
| ATOM | 39 | H39 | URI | 1 | 3.744 | −0.164 | −1.270 | −0.18 | −0.19 | +0.424 | 3.256 |
| ATOM | 40 | O40 | URI | 1 | 1.449 | −1.367 | −1.900 | +0.08 | −0.10 | −0.227 | 3.256 |
| ATOM | 41 | C12 | URI | 1 | −0.993 | −7.525 | −3.486 | −0.25 | −0.04 | +0.113 | 3.256 |
| ATOM | 42 | O15 | URI | 1 | −2.179 | −7.026 | −2.859 | +0.16 | −0.16 | −0.537 | 3.256 |
| ATOM | 43 | H15 | URI | 1 | −1.867 | −6.382 | −2.196 | +0.11 | +0.09 | +0.424 | 3.256 |
| ATOM | 44 | C10 | URI | 1 | −1.365 | −8.870 | −4.150 | −0.30 | +0.05 | +0.113 | 3.256 |
| ATOM | 45 | O11 | URI | 1 | −2.341 | −9.533 | −3.380 | +0.12 | −0.28 | −0.537 | 3.256 |
| ATOM | 46 | H11 | URI | 1 | −1.832 | −10.169 | −2.836 | +0.09 | +0.21 | +0.424 | 3.256 |
| TER | | | | | | | | | | | |
| ENDMDL | | | | | | | | | | | |
| MODEL | 11 | | | | | | | | | | |
| USER | Run = 11 | | | | | | | | | | |
| USER | Cluster Rank = 13 | | | | | | | | | | |
| USER | Number of conformations in this cluster = 1 | | | | | | | | | | |
| USER | | | | | | | | | | | |
| USER | RMSD from reference structure = 6.215 A | | | | | | | | | | |
| USER | | | | | | | | | | | |
| USER | Estimated Free Energy of Binding = −5.39 kcal/mol [= (1) + (3)] | | | | | | | | | | |
| USER | Estimated Inhibition Constant, Ki = +1.12e−04 [Temperature = 298.15 K] | | | | | | | | | | |
| USER | | | | | | | | | | | |
| USER | Final Docked Energy = −9.21 kcal/mol [= (1) + (2)] | | | | | | | | | | |
| USER | | | | | | | | | | | |
| USER | (1) Final Intermolecular Energy = −10.06 kcal/mol | | | | | | | | | | |
| USER | (2) Final Internal Energy of Ligand = +0.85 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −1.854730 −9.249351 −3.853351 | | | | | | | | | | |
| USER | NEWDPF quat0 0.449693 −0.319549 0.834065 −116.092132 | | | | | | | | | | |
| USER | NEWDPF ndihe 15 | | | | | | | | | | |
| USER | NEWDPF dihe0 −104.47 164.55 −51.62 114.60 148.94 140.39 −21.81 11.02 | | | | | | | | | | |
| | 18.06 −156.47 −103.77 −50.90 −129.20 −34.16 36.21 | | | | | | | | | | |
| USER | | | | | | | | | | | |
| USER | | | | | x | y | z | vdW | Elec | q | RMS |
| ATOM | 1 | C2 | URI | 1 | −7.069 | −4.516 | 0.753 | −0.74 | −0.18 | +0.296 | 6.215 |
| ATOM | 2 | N3 | URI | 1 | −7.176 | −4.225 | 2.090 | −0.55 | +0.17 | −0.292 | 6.215 |
| ATOM | 3 | C4 | URI | 1 | −6.148 | −4.281 | 3.032 | −0.68 | −0.11 | +0.346 | 6.215 |
| ATOM | 4 | C5 | URI | 1 | −4.885 | −4.700 | 2.509 | −0.40 | +0.00 | +0.000 | 6.215 |
| ATOM | 5 | C6 | URI | 1 | −4.769 | −4.982 | 1.203 | −0.32 | +0.02 | −0.050 | 6.215 |
| ATOM | 6 | N1 | URI | 1 | −5.838 | −4.917 | 0.315 | −0.29 | −0.02 | +0.039 | 6.215 |
| ATOM | 7 | O8 | URI | 1 | −6.413 | −3.994 | 4.199 | −0.42 | −0.08 | −0.396 | 6.215 |
| ATOM | 8 | O7 | URI | 1 | −8.035 | −4.433 | −0.007 | −0.19 | +0.29 | −0.396 | 6.215 |
| ATOM | 9 | H3 | URI | 1 | −8.082 | −3.945 | 2.421 | −0.25 | −0.28 | +0.393 | 6.215 |
| ATOM | 10 | C9 | URI | 1 | −5.657 | −5.258 | −1.101 | −0.48 | −0.04 | +0.174 | 6.215 |
| ATOM | 11 | O14 | URI | 1 | −6.092 | −6.604 | −1.246 | +0.08 | −0.01 | −0.227 | 6.215 |
| ATOM | 12 | C13 | URI | 1 | −5.481 | −7.179 | −2.433 | −0.37 | 0.04 | +0.113 | 6.215 |
| ATOM | 13 | C16 | URI | 1 | −5.009 | −8.595 | −2.100 | −0.22 | +0.05 | +0.113 | 6.215 |
| ATOM | 14 | O17 | URI | 1 | −5.212 | −9.401 | −3.245 | +0.09 | −0.37 | −0.368 | 6.215 |
| ATOM | 15 | P18 | URI | 1 | −4.377 | −9.641 | −4.562 | −0.39 | +1.23 | +1.210 | 6.215 |
| ATOM | 16 | P19 | URI | 1 | −5.218 | −10.444 | −5.495 | −0.07 | −1.07 | −0.850 | 6.215 |
| ATOM | 17 | O20 | URI | 1 | −3.852 | − 8.322 | −5.039 | −0.09 | −0.93 | −0.850 | 6.215 |
| ATOM | 18 | O21 | URI | 1 | −3.279 | −10.577 | −3.883 | +0.03 | −0.39 | −0.510 | 6.215 |
| ATOM | 19 | P22 | URI | 1 | −3.372 | −11.862 | −2.937 | −0.28 | +0.77 | +1.210 | 6.215 |
| ATOM | 20 | O25 | URI | 1 | −4.687 | −12.552 | −2.964 | +0.10 | −0.55 | −0.850 | 6.215 |
| ATOM | 21 | O24 | URI | 1 | −2.138 | −12.589 | −3.328 | −0.01 | −0.57 | −0.850 | 6.215 |
| ATOM | 22 | O23 | URI | 1 | −3.265 | −11.275 | −1.436 | +0.16 | −0.15 | −0.368 | 6.215 |
| ATOM | 23 | C37 | URI | 1 | −1.882 | −11.391 | −1.184 | −0.34 | +0.10 | +0.227 | 6.215 |
| ATOM | 24 | C45 | URI | 1 | −1.604 | −11.609 | 0.336 | −0.29 | +0.08 | +0.211 | 6.215 |
| ATOM | 25 | N41 | URI | 1 | −2.400 | −12.770 | 0.822 | −0.18 | −0.23 | −0.650 | 6.215 |
| ATOM | 26 | H41 | URI | 1 | −3.377 | −12.774 | 0.531 | +0.11 | +0.14 | +0.440 | 6.215 |
| ATOM | 27 | C49 | URI | 1 | −1.873 | −13.788 | 1.510 | −0.38 | +0.17 | +0.396 | 6.215 |
| ATOM | 28 | O38 | URI | 1 | −0.695 | −13.842 | 1.868 | −0.27 | −0.24 | −0.396 | 6.215 |
| ATOM | 29 | C50 | URI | 1 | −2.833 | −14.926 | 1.843 | −0.28 | +0.00 | +0.000 | 6.215 |
| ATOM | 30 | C44 | URI | 1 | −1.892 | −10.298 | 1.132 | −0.20 | +0.03 | +0.113 | 6.215 |
| ATOM | 31 | O47 | URI | 1 | −1.461 | −10.437 | 2.483 | +0.12 | −0.11 | −0.537 | 6.215 |
| ATOM | 32 | H47 | URI | 1 | −2.248 | −10.326 | 3.057 | +0.11 | +0.07 | +0.424 | 6.215 |
| ATOM | 33 | C43 | URI | 1 | −1.182 | −9.064 | 0.520 | −0.22 | +0.02 | +0.113 | 6.215 |
| ATOM | 34 | O48 | URI | 1 | −1.613 | −7.897 | 1.210 | +0.18 | −0.07 | −0.537 | 6.215 |
| ATOM | 35 | H48 | URI | 1 | −1.518 | −7.174 | 0.558 | +0.11 | +0.05 | +0.424 | 6.215 |
| ATOM | 36 | C46 | URI | 1 | −1.490 | −8.943 | −0.998 | −0.24 | +0.03 | +0.113 | 6.215 |
| ATOM | 37 | C42 | URI | 1 | −0.693 | −7.830 | −1.697 | −0.26 | +0.03 | +0.113 | 6.215 |
| ATOM | 38 | O39 | URI | 1 | −1.312 | −7.502 | −2.936 | +0.14 | −0.17 | −0.537 | 6.215 |
| ATOM | 39 | H39 | URI | 1 | −1.443 | −6.527 | −2.960 | +0.11 | +0.12 | +0.424 | 6.215 |
| ATOM | 40 | O40 | URI | 1 | −1.180 | −10.205 | −1.663 | +0.02 | −0.10 | −0.227 | 6.215 |
| ATOM | 41 | C12 | URI | 1 | −4.336 | −6.237 | −2.799 | −0.35 | +0.02 | +0.113 | 6.215 |
| ATOM | 42 | O15 | URI | 1 | −4.731 | −5.399 | −3.891 | +0.05 | −0.07 | −0.537 | 6.215 |
| ATOM | 43 | H15 | URI | 1 | −5.686 | −5.250 | −3.765 | +0.03 | −0.01 | +0.424 | 6.215 |
| ATOM | 44 | C10 | URI | 1 | −4.198 | −5.326 | −1.558 | −0.32 | −0.01 | +0.113 | 6.215 |
| ATOM | 45 | O11 | URI | 1 | −3.741 | −4.051 | −1.946 | +0.16 | +0.17 | −0.537 | 6.215 |
| ATOM | 46 | H11 | URI | 1 | −3.785 | −3.516 | −1.126 | −0.17 | −0.29 | +0.424 | 6.215 |
| TER | | | | | | | | | | | |
| ENDMDL | | | | | | | | | | | |
| MODEL | 27 | | | | | | | | | | |
| USER | Run = 27 | | | | | | | | | | |
| USER | Cluster Rank = 14 | | | | | | | | | | |
| USER | Number of conformations in this cluster = 1 | | | | | | | | | | |
| USER | | | | | | | | | | | |
| USER | RMSD from reference structure = 2.640 A | | | | | | | | | | |
| USER | | | | | | | | | | | |
| USER | Estimated Free Energy of Binding = −5.76 kcal/mol [= (1) + (3)] | | | | | | | | | | |
| USER | Estimated Inhibition Constant, Ki = +5.96e−05 [Temperature = 298.15 K] | | | | | | | | | | |
| USER | | | | | | | | | | | |
| USER | Final Docked Energy = −9.20 kcal/mol [= (1) + (2)] | | | | | | | | | | |
| USER | | | | | | | | | | | |
| USER | (1) Final Intermolecular Energy = −10.43 kcal/mol | | | | | | | | | | |
| USER | (2) Final Internal Energy of Ligand = +1.23 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 0.509397 −3.452187 −2.565839 | | | | | | | | | | |
| USER | NEWDPF quat0 0.952279 0.192807 0.236624 14.672749 | | | | | | | | | | |
| USER | NEWDPF ndihe 15 | | | | | | | | | | |
| USER | NEWDPF dihe0 −47.35 −90.76 −28.98 −19.78 44.27 −166.76 −30.49 30.92 | | | | | | | | | | |
| | −17.35 4.18 104.63 −86.18 −27.11 −120.42 29.50 | | | | | | | | | | |
| USER | | | | | | | | | | | |
| USER | | | | | x | y | z | vdW | Elec | q | RMS |
| ATOM | 1 | C2 | URI | 1 | −1.735 | −10.827 | −5.940 | −0.57 | +0.23 | +0.296 | 2.640 |
| ATOM | 2 | N3 | URI | 1 | −1.557 | −12.140 | −5.586 | −0.44 | −0.24 | −0.292 | 2.640 |
| ATOM | 3 | C4 | URI | 1 | −1.332 | −12.625 | −4.296 | −0.66 | +0.24 | +0.346 | 2.640 |
| ATOM | 4 | C5 | URI | 1 | −1.277 | −11.616 | −3.285 | −0.43 | +0.00 | +0.000 | 2.640 |
| ATOM | 5 | C6 | URI | 1 | −1.454 | −10.330 | −3.621 | −0.25 | −0.03 | −0.050 | 2.640 |
| ATOM | 6 | N1 | URI | 1 | −1.665 | −9.910 | −4.929 | −0.24 | +0.02 | +0.039 | 2.640 |
| ATOM | 7 | O8 | URI | 1 | −1.186 | −13.838 | −4.149 | −0.26 | −0.29 | −0.396 | 2.640 |
| ATOM | 8 | O7 | URI | 1 | −1.934 | −10.492 | −7.109 | −0.10 | −0.27 | −0.396 | 2.640 |
| ATOM | 9 | H3 | URI | 1 | −1.593 | −1.818 | −6.327 | −0.17 | +0.19 | +0.393 | 2.640 |
| ATOM | 10 | C9 | URI | 1 | −1.831 | −8.485 | −5.241 | −0.35 | +0.10 | +0.174 | 2.640 |
| ATOM | 11 | O14 | URI | 1 | −0.531 | −7.985 | −5.531 | +0.06 | −0.09 | −0.227 | 2.640 |
| ATOM | 12 | C13 | URI | 1 | −0.517 | −6.545 | −5.334 | −0.26 | +0.01 | +0.113 | 2.640 |
| ATOM | 13 | C16 | URI | 1 | 0.772 | −6.167 | −4.602 | −0.30 | +0.04 | +0.113 | 2.640 |
| ATOM | 14 | O17 | URI | 1 | 1.862 | −6.622 | −5.381 | +0.04 | −0.18 | −0.368 | 2.640 |
| ATOM | 15 | P18 | URI | 1 | 3.423 | −6.640 | −5.154 | −0.60 | +0.95 | +1.210 | 2.640 |
| ATOM | 16 | O19 | URI | 1 | 3.987 | −7.765 | −5.953 | −0.16 | −0.62 | −0.850 | 2.640 |
| ATOM | 17 | O20 | URI | 1 | 3.953 | −5.261 | −5.400 | −0.02 | −0.94 | −0.850 | 2.640 |
| ATOM | 18 | O21 | URI | 1 | 3.353 | −7.051 | −3.615 | +0.10 | −0.39 | −0.510 | 2.640 |
| ATOM | 19 | P22 | URI | 1 | 3.514 | −6.227 | −2.255 | −0.51 | +1.03 | +1.210 | 2.640 |
| ATOM | 20 | O25 | URI | 1 | 3.357 | −7.038 | −1.022 | +0.05 | −0.51 | −0.850 | 2.640 |
| ATOM | 21 | O24 | URI | 1 | 4.781 | −5.499 | −2.515 | −0.33 | −1.52 | −0.850 | 2.640 |
| ATOM | 22 | O23 | URI | 1 | 2.248 | −5.224 | −2.231 | +0.09 | −0.21 | −0.368 | 2.640 |
| ATOM | 23 | C37 | URI | 1 | 2.872 | −3.985 | −1.977 | −0.04 | +0.16 | +0.227 | 2.640 |
| ATOM | 24 | C45 | URI | 1 | 2.168 | −3.231 | −0.807 | −0.21 | +0.08 | +0.211 | 2.640 |
| ATOM | 25 | N41 | URI | 1 | 2.100 | −4.121 | 0.385 | +0.11 | −0.20 | −0.650 | 2.640 |
| ATOM | 26 | H41 | URI | 1 | 1.652 | −5.023 | 0.223 | −0.11 | +0.13 | +0.440 | 2.640 |
| ATOM | 27 | C49 | URI | 1 | 2.660 | −3.822 | 1.562 | −0.18 | +0.10 | +0.396 | 2.640 |
| ATOM | 28 | O38 | URI | 1 | 3.231 | −2.757 | 1.802 | +0.14 | −0.08 | −0.396 | 2.640 |
| ATOM | 29 | C50 | URI | 1 | 2.558 | −4.902 | 2.635 | −0.15 | +0.00 | +0.000 | 2.640 |
| ATOM | 30 | C44 | URI | 1 | 0.768 | −2.714 | −1.260 | −0.19 | +0.04 | +0.113 | 2.640 |
| ATOM | 31 | O47 | URI | 1 | 0.206 | −1.868 | −0.261 | +0.16 | −0.04 | −0.537 | 2.640 |
| ATOM | 32 | H47 | URI | 1 | 0.087 | −2.406 | 0.550 | +0.11 | −0.01 | +0.424 | 2.640 |
| ATOM | 33 | C43 | URI | 1 | 0.829 | −1.920 | −2.590 | −0.31 | +0.11 | +0.113 | 2.640 |
| ATOM | 34 | O48 | URI | 1 | −0.497 | −1.617 | −3.007 | −0.32 | −1.13 | −0.537 | 2.640 |
| ATOM | 35 | H48 | URI | 1 | −0.979 | −2.465 | −2.932 | +0.08 | +0.69 | +0.424 | 2.640 |
| ATOM | 36 | C46 | URI | 1 | 1.572 | −2.733 | −3.687 | −0.33 | +0.14 | +0.113 | 2.640 |
| ATOM | 37 | C42 | URI | 1 | 1.803 | −1.941 | −4.984 | −0.55 | +0.17 | +0.113 | 2.640 |
| ATOM | 38 | O39 | URI | 1 | 0.961 | −2.450 | −6.013 | −0.39 | −0.72 | −0.537 | 2.640 |
| ATOM | 39 | H39 | URI | 1 | 0.763 | −1.711 | −6.633 | +0.03 | +0.58 | +0.424 | 2.640 |
| ATOM | 40 | O40 | URI | 1 | 2.871 | −3.163 | −3.183 | +0.07 | −0.18 | −0.227 | 2.640 |
| ATOM | 41 | C12 | URI | 1 | −1.777 | −6.232 | −4.527 | −0.32 | +0.03 | +0.113 | 2.640 |
| ATOM | 42 | O15 | URI | 1 | −2.786 | −5.706 | −5.394 | +0.00 | −0.13 | −0.537 | 2.640 |
| ATOM | 43 | H15 | URI | 1 | −2.766 | −4.741 | −5.254 | +0.02 | −0.08 | +0.424 | 2.640 |
| ATOM | 44 | C10 | URI | 1 | −2.266 | −7.621 | −4.055 | −0.26 | +0.05 | +0.113 | 2.640 |
| ATOM | 45 | O11 | URI | 1 | −3.669 | −7.620 | −3.932 | +0.04 | −0.34 | −0.537 | 2.640 |
| ATOM | 46 | H11 | URI | 1 | −3.995 | −7.551 | −4.853 | +0.08 | +0.51 | +0.424 | 2.640 |
| TER | | | | | | | | | | | |
| ENDMDL | | | | | | | | | | | |
| MODEL | 9 | | | | | | | | | | |
| USER | Run = 9 | | | | | | | | | | |
| USER | Cluster Rank = 15 | | | | | | | | | | |
| USER | Number of conformations in this cluster = 1 | | | | | | | | | | |
| USER | | | | | | | | | | | |
| USER | RMSD from reference structure = 3.504 A | | | | | | | | | | |
| USER | | | | | | | | | | | |
| USER | Estimated Free Energy of Binding = −5.27 kcal/mol [= (1) + (3)] | | | | | | | | | | |
| USER | Estimated Inhibition Constant, Ki = +1.37e−04 [Temperature = 298.15 K] | | | | | | | | | | |
| USER | | | | | | | | | | | |
| USER | Final Docked Energy = −9.07 kcal/mol [= (1) + (2)] | | | | | | | | | | |
| USER | | | | | | | | | | | |
| USER | (1) Final Intermolecular Energy = −9.94 kcal/mol | | | | | | | | | | |
| USER | (2) Final Internal Energy of Ligand = +0.87 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.744622 −4.517434 −3.381963 | | | | | | | | | | |
| USER | NEWDPF quat0 0.653836 0.651103 −0.385440 13.824376 | | | | | | | | | | |
| USER | NEWDPF ndihe 15 | | | | | | | | | | |
| USER | NEWDPF dihe0 −114.67 10.05 −26.09 7.71 52.91 −150.08 −62.58 51.17 | | | | | | | | | | |
| | 2.01 −125.85 −40.31 54.53 14.54 3.71 1.79 | | | | | | | | | | |
| USER | | | | | | | | | | | |
| USER | | | | | x | y | z | vdW | Elec | q | RMS |
| ATOM | 1 | C2 | URI | 1 | −1.857 | −11.755 | −5.718 | −0.42 | +0.26 | +0.296 | 3.504 |
| ATOM | 2 | N3 | URI | 1 | −1.808 | −13.046 | −5.257 | −0.23 | −0.21 | −0.292 | 3.504 |
| ATOM | 3 | C4 | URI | 1 | −1.484 | −13.448 | −3.960 | −0.68 | +0.24 | +0.346 | 3.504 |
| ATOM | 4 | C5 | URI | 1 | −1.168 | −12.377 | −3.067 | −0.58 | +0.00 | +0.000 | 3.504 |
| ATOM | 5 | C6 | URI | 1 | −1.218 | −11.111 | −3.508 | −0.22 | −0.03 | −0.050 | 3.504 |
| ATOM | 6 | N1 | URI | 1 | −1.538 | −10.776 | −4.819 | −0.30 | +0.03 | +0.039 | 3.504 |
| ATOM | 7 | O8 | URI | 1 | −1.480 | −14.653 | −3.709 | −0.23 | −0.34 | −0.396 | 3.504 |
| ATOM | 8 | O7 | URI | 1 | −2.159 | −11.495 | −6.883 | −0.23 | −0.31 | −0.396 | 3.504 |
| ATOM | 9 | H3 | URI | 1 | −2.026 | −13.772 | −5.916 | −0.24 | +0.00 | +0.393 | 3.504 |
| ATOM | 10 | C9 | URI | 1 | −1.556 | −9.373 | −5.251 | −0.40 | +0.10 | +0.174 | 3.504 |
| ATOM | 11 | O14 | URI | 1 | −0.223 | −8.896 | −5.113 | +0.07 | −0.12 | −0.227 | 3.504 |
| ATOM | 12 | C13 | URI | 1 | −0.242 | −7.447 | −5.006 | −0.34 | +0.04 | +0.113 | 3.504 |
| ATOM | 13 | C16 | URI | 1 | 0.734 | −7.027 | −3.906 | −0.33 | +0.04 | +0.113 | 3.504 |
| ATOM | 14 | O17 | URI | 1 | 0.674 | −5.618 | −3.788 | +0.12 | −0.16 | −0.368 | 3.504 |
| ATOM | 15 | P18 | URI | 1 | 1.774 | −4.492 | −3.686 | −0.26 | +0.84 | +1.210 | 3.504 |
| ATOM | 16 | O19 | URI | 1 | 2.501 | −4.680 | −2.399 | +0.09 | −0.56 | −0.850 | 3.504 |
| ATOM | 17 | O20 | URI | 1 | 2.569 | −4.489 | −4.955 | +0.09 | −0.68 | −0.850 | 3.504 |
| ATOM | 18 | O21 | URI | 1 | 0.745 | −3.280 | −3.554 | −0.21 | −0.70 | −0.510 | 3.504 |
| ATOM | 19 | P22 | URI | 1 | 0.402 | −2.262 | −2.371 | −0.24 | +1.16 | +1.210 | 3.504 |
| ATOM | 20 | O25 | URI | 1 | 1.587 | −1.611 | −1.756 | +0.09 | −0.36 | −0.850 | 3.504 |
| ATOM | 21 | O24 | URI | 1 | −0.657 | −1.439 | −3.007 | −0.37 | −1.65 | −0.850 | 3.504 |
| ATOM | 22 | O23 | URI | 1 | −0.195 | −3.189 | −1.192 | +0.16 | −0.11 | −0.368 | 3.504 |
| ATOM | 23 | C37 | URI | 1 | −1.554 | −2.815 | −1.197 | −0.23 | +0.00 | +0.227 | 3.504 |
| ATOM | 24 | C45 | URI | 1 | −1.918 | −2.003 | 0.085 | −0.26 | −0.08 | +0.211 | 3.504 |
| ATOM | 25 | N41 | URI | 1 | −0.983 | −0.853 | 0.224 | −0.20 | +0.18 | −0.650 | 3.504 |
| ATOM | 26 | H41 | URI | 1 | −0.118 | −1.060 | 0.725 | +0.11 | −0.09 | +0.440 | 3.504 |
| ATOM | 27 | C49 | URI | 1 | −1.195 | 0.342 | −0.336 | −0.48 | −0.11 | +0.396 | 3.504 |
| ATOM | 28 | O38 | URI | 1 | −2.211 | 0.636 | −0.968 | −0.10 | +0.13 | −0.396 | 3.504 |
| ATOM | 29 | C50 | URI | 1 | −0.078 | 1.365 | −0.153 | −0.40 | +0.00 | +0.000 | 3.504 |
| ATOM | 30 | C44 | URI | 1 | −1.924 | −2.938 | 1.334 | −0.29 | −0.04 | +0.113 | 3.504 |
| ATOM | 31 | O47 | URI | 1 | −2.438 | −2.243 | 2.468 | −0.03 | +0.37 | −0.537 | 3.504 |
| ATOM | 32 | H47 | URI | 1 | −1.801 | −2.365 | 3.203 | +0.08 | −0.23 | +0.424 | 3.504 |
| ATOM | 33 | C43 | URI | 1 | −2.778 | −4.213 | 1.119 | −0.24 | −0.03 | +0.113 | 3.504 |
| ATOM | 34 | O48 | URI | 1 | −2.609 | −5.075 | 2.238 | +0.17 | +0.07 | −0.537 | 3.504 |
| ATOM | 35 | H48 | URI | 1 | −2.804 | −4.513 | 3.015 | +0.11 | −0.08 | +0.424 | 3.504 |
| ATOM | 36 | C46 | URI | 1 | −2.368 | −4.939 | −0.193 | −0.16 | −0.01 | +0.113 | 3.504 |
| ATOM | 37 | C42 | URI | 1 | −3.279 | −6.126 | −0.545 | −0.14 | +0.00 | +0.113 | 3.504 |
| ATOM | 38 | O39 | URI | 1 | −2.740 | −7.324 | 0.003 | +0.21 | −0.06 | −0.537 | 3.504 |
| ATOM | 39 | H39 | URI | 1 | −3.484 | −7.953 | 0.144 | +0.12 | +0.06 | +0.424 | 3.504 |
| ATOM | 40 | O40 | URI | 1 | −2.403 | −3.997 | −1.306 | +0.10 | +0.02 | −0.227 | 3.504 |
| ATOM | 41 | C12 | URI | 1 | −1.692 | −7.080 | −4.692 | −0.31 | +0.04 | +0.113 | 3.504 |
| ATOM | 42 | O15 | URI | 1 | −2.337 | −6.606 | −5.878 | −0.23 | −0.23 | −0.537 | 3.504 |
| ATOM | 43 | H15 | URI | 1 | −1.970 | −5.715 | −6.030 | −0.10 | −0.23 | +0.424 | 3.504 |
| ATOM | 44 | C10 | URI | 1 | −2.344 | −8.435 | −4.334 | −0.29 | +0.06 | +0.113 | 3.504 |
| ATOM | 45 | O11 | URI | 1 | −3.707 | −8.422 | −4.691 | −0.04 | −0.49 | −0.537 | 3.504 |
| ATOM | 46 | H11 | URI | 1 | −3.705 | −8.553 | −5.662 | +0.08 | +0.49 | +0.424 | 3.504 |
| TER | | | | | | | | | | | |
| ENDMDL | | | | | | | | | | | |
| MODEL | 45 | | | | | | | | | | |
| USER | Run = 45 | | | | | | | | | | |
| USER | Cluster Rank = 16 | | | | | | | | | | |
| USER | Number of conformations in this cluster = 1 | | | | | | | | | | |
| USER | | | | | | | | | | | |
| USER | RMSD from reference structure = 4.106 A | | | | | | | | | | |
| USER | | | | | | | | | | | |
| USER | Estimated Free Energy of Binding = −5.33 kcal/mol [= (1) + (3)] | | | | | | | | | | |
| USER | Estimated Inhibition Constant, Ki = +1.23e−04 [Temperature = 298.15 K] | | | | | | | | | | |
| USER | | | | | | | | | | | |
| USER | Final Docked Energy = −9.00 kcal/mol [= (1) + (2)] | | | | | | | | | | |
| USER | | | | | | | | | | | |
| USER | (1) Final Intermolecular Energy = −10.00 kcal/mol | | | | | | | | | | |
| USER | (2) Final Internal Energy of Ligand = +1.01 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 0.961640 −3.486293 0.100002 | | | | | | | | | | |
| USER | NEWDPF quat0 0.220305 0.456224 0.862163 −60.001263 | | | | | | | | | | |
| USER | NEWDPF ndihe 15 | | | | | | | | | | |
| USER | NEWDPF dihe0 −179.36 129.92 −67.45 −129.17 125.46 122.82 −69.41 23.36 13.28 | | | | | | | | | | |
| | 154.17 −4.39 152.02 −145.72 −48.09 −12.93 | | | | | | | | | | |
| USER | | | | | | | | | | | |
| USER | | | | | x | y | z | vdW | Elec | q | RMS |
| ATOM | 1 | C2 | URI | 1 | −6.153 | −7.054 | −2.632 | −0.47 | +0.12 | +0.296 | 4.106 |
| ATOM | 2 | N3 | URI | 1 | −7.267 | −7.571 | −2.020 | −0.30 | −0.09 | −0.292 | 4.106 |
| ATOM | 3 | C4 | URI | 1 | −7.741 | −7.235 | −0.751 | −0.44 | −0.03 | +0.346 | 4.106 |
| ATOM | 4 | C5 | URI | 1 | −6.951 | −6.267 | −0.058 | −0.42 | +0.00 | +0.000 | 4.106 |
| ATOM | 5 | C6 | URI | 1 | −5.863 | −5.753 | −0.650 | −0.39 | +0.01 | −0.050 | 4.106 |
| ATOM | 6 | N1 | URI | 1 | −5.435 | −6.138 | −1.916 | −0.23 | +0.00 | +0.039 | 4.106 |
| ATOM | 7 | O8 | URI | 1 | −8.757 | −7.798 | −0.343 | −0.01 | +0.04 | −0.396 | 4.106 |
| ATOM | 8 | O7 | URI | 1 | −5.812 | −7.409 | −3.762 | −0.17 | −0.43 | −0.396 | 4.106 |
| ATOM | 9 | H3 | URI | 1 | −7.790 | −8.256 | −2.534 | +0.08 | +0.22 | +0.393 | 4.106 |
| ATOM | 10 | C9 | URI | 1 | −4.223 | −5.557 | −2.507 | −0.38 | +0.01 | +0.174 | 4.106 |
| ATOM | 11 | O14 | URI | 1 | −3.211 | −5.645 | −1.511 | +0.15 | −0.01 | −0.227 | 4.106 |
| ATOM | 12 | C13 | URI | 1 | −2.179 | −4.660 | −1.789 | −0.23 | +0.01 | +0.113 | 4.106 |
| ATOM | 13 | C16 | URI | 1 | −1.781 | −3.988 | −0.474 | −0.20 | −0.01 | +0.113 | 4.106 |
| ATOM | 14 | O17 | URI | 1 | −0.370 | −4.027 | −0.377 | +0.19 | −0.05 | −0.368 | 4.106 |
| ATOM | 15 | P18 | URI | 1 | 0.792 | −3.350 | −1.201 | −0.15 | +0.44 | +1.210 | 4.106 |
| ATOM | 16 | O19 | URI | 1 | 0.296 | −2.032 | −1.689 | +0.10 | −0.44 | −0.850 | 4.106 |
| ATOM | 17 | O20 | URI | 1 | 2.039 | −3.374 | −0.372 | +0.14 | −0.27 | −0.850 | 4.106 |
| ATOM | 18 | O21 | URI | 1 | 0.776 | −4.409 | −2.393 | +0.16 | −0.27 | −0.510 | 4.106 |
| ATOM | 19 | P22 | URI | 1 | 1.105 | −4.303 | −3.954 | −0.25 | +0.91 | +1.210 | 4.106 |
| ATOM | 20 | O25 | URI | 1 | 0.817 | −2.976 | −4.555 | −0.21 | −1.79 | −0.850 | 4.106 |
| ATOM | 21 | O24 | URI | 1 | 2.482 | −4.858 | −3.989 | +0.10 | −0.62 | −0.850 | 4.106 |
| ATOM | 22 | O23 | URI | 1 | 0.047 | −5.295 | −4.664 | +0.08 | −0.10 | −0.368 | 4.106 |
| ATOM | 23 | C37 | URI | 1 | 0.828 | −6.453 | −4.856 | −0.33 | +0.08 | +0.227 | 4.106 |
| ATOM | 24 | C45 | URI | 1 | 0.570 | −7.078 | −6.262 | −0.43 | +0.06 | +0.211 | 4.106 |
| ATOM | 25 | N41 | URI | 1 | 0.771 | −6.040 | −7.310 | −0.21 | −0.05 | −0.650 | 4.106 |
| ATOM | 26 | H41 | URI | 1 | −0.075 | −5.551 | −7.600 | −0.21 | −0.35 | +0.440 | 4.106 |
| ATOM | 27 | C49 | URI | 1 | 1.973 | −5.693 | −7.782 | −0.56 | +0.20 | +0.396 | 4.106 |
| ATOM | 28 | O38 | URI | 1 | 3.027 | −6.237 | −7.449 | +0.00 | −0.30 | −0.396 | 4.106 |
| ATOM | 29 | C50 | URI | 1 | 1.981 | −4.546 | −8.789 | −0.70 | +0.00 | +0.000 | 4.106 |
| ATOM | 30 | C44 | URI | 1 | −0.849 | −7.727 | −6.313 | −0.43 | +0.04 | +0.113 | 4.106 |
| ATOM | 31 | O47 | URI | 1 | −1.014 | −8.459 | −7.525 | −0.01 | −0.26 | −0.537 | 4.106 |
| ATOM | 32 | H47 | URI | 1 | −1.806 | −9.027 | −7.422 | +0.08 | +0.24 | +0.424 | 4.106 |
| ATOM | 33 | C43 | URI | 1 | −1.106 | −8.689 | −5.126 | −0.37 | +0.06 | +0.113 | 4.106 |
| ATOM | 34 | O48 | URI | 1 | −2.462 | −9.116 | −5.165 | +0.06 | −0.36 | −0.537 | 4.106 |
| ATOM | 35 | H48 | URI | 1 | −2.525 | −9.805 | −4.474 | +0.10 | +0.28 | +0.424 | 4.106 |
| ATOM | 36 | C46 | URI | 1 | −0.794 | −7.993 | −3.772 | −0.28 | +0.04 | +0.113 | 4.106 |
| ATOM | 37 | C42 | URI | 1 | −0.880 | −8.940 | −2.564 | −0.24 | +0.04 | +0.113 | 4.106 |
| ATOM | 38 | O39 | URI | 1 | −0.596 | −8.221 | −1.369 | +0.05 | −0.13 | −0.537 | 4.106 |
| ATOM | 39 | H39 | URI | 1 | −1.423 | −8.181 | −0.837 | +0.11 | +0.09 | +0.424 | 4.106 |
| ATOM | 40 | O40 | URI | 1 | 0.551 | −7.430 | −3.809 | +0.05 | −0.09 | −0.227 | 4.106 |
| ATOM | 41 | C12 | URI | 1 | −2.800 | −3.690 | −2.793 | −0.26 | +0.00 | +0.113 | 4.106 |
| ATOM | 42 | O15 | URI | 1 | −2.319 | −3.985 | −4.108 | −0.08 | −0.10 | −0.537 | 4.106 |
| ATOM | 43 | H15 | URI | 1 | −3.045 | −3.731 | −4.708 | −0.04 | −0.05 | +0.424 | 4.106 |
| ATOM | 44 | C10 | URI | 1 | −4.303 | −4.051 | −2.771 | −0.32 | −0.02 | +0.113 | 4.106 |
| ATOM | 45 | O11 | URI | 1 | −4.877 | −3.799 | −4.033 | −0.16 | +0.00 | −0.537 | 4.106 |
| ATOM | 46 | H11 | URI | 1 | −5.600 | −4.456 | −4.107 | +0.01 | −0.04 | +0.424 | 4.106 |
| TER | | | | | | | | | | | |
| ENDMDL | | | | | | | | | | | |
| MODEL | 15 | | | | | | | | | | |
| USER | Run = 15 | | | | | | | | | | |
| USER | Cluster Rank = 17 | | | | | | | | | | |
| USER | Number of conformations in this cluster = 1 | | | | | | | | | | |
| USER | | | | | | | | | | | |
| USER | RMSD from reference structure = 2.484 A | | | | | | | | | | |
| USER | | | | | | | | | | | |
| USER | Estimated Free Energy of Binding = −7.26 kcal/mol [= (1) + (3)] | | | | | | | | | | |
| USER | Estimated Inhibition Constant, Ki = +4.81e−06 [Temperature = 298.15 K] | | | | | | | | | | |
| USER | | | | | | | | | | | |
| USER | Final Docked Energy = −8.64 kcal/mol [= (1) + (2)] | | | | | | | | | | |
| USER | | | | | | | | | | | |
| USER | (1) Final Intermolecular Energy = −11.92 kcal/mol | | | | | | | | | | |
| USER | (2) Final Internal Energy of Ligand = +3.29 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −0.658315 −5.012790 −2.126138 | | | | | | | | | | |
| USER | NEWDPF quat0 0.010389 0.987568 −0.156848 −28.123127 | | | | | | | | | | |
| USER | NEWDPF ndihe 15 | | | | | | | | | | |
| USER | NEWDPF dihe0 −102.34 139.37 −141.35 42.13 −90.11 −176.24 127.88 36.20 20.36 | | | | | | | | | | |
| | −147.83 49.18 −148.48 78.68 27.01 −19.80 | | | | | | | | | | |
| USER | | | | | | | | | | | |
| USER | | | | | x | y | z | vdW | Elec | q | RMS |
| ATOM | 1 | C2 | URI | 1 | −1.608 | −13.003 | −4.588 | −0.63 | +0.21 | +0.296 | 2.484 |
| ATOM | 2 | N3 | URI | 1 | −1.765 | −14.199 | −3.934 | −0.45 | −0.23 | −0.292 | 2.484 |
| ATOM | 3 | C4 | URI | 1 | −2.273 | −14.375 | −2.645 | −0.39 | +0.24 | +0.346 | 2.484 |
| ATOM | 4 | C5 | URI | 1 | −2.637 | −13.163 | −1.980 | −0.34 | +0.00 | +0.000 | 2.484 |
| ATOM | 5 | C6 | URI | 1 | −2.491 | −11.990 | −2.613 | −0.36 | −0.03 | −0.050 | 2.484 |
| ATOM | 6 | N1 | URI | 1 | −1.968 | −11.880 | −3.897 | −0.31 | +0.03 | +0.039 | 2.484 |
| ATOM | 7 | O8 | URI | 1 | −2.342 | −15.520 | −2.200 | +0.07 | −0.27 | −0.396 | 2.484 |
| ATOM | 8 | O7 | URI | 1 | −1.155 | −12.946 | −5.732 | −0.06 | −0.28 | −0.396 | 2.484 |
| ATOM | 9 | H3 | URI | 1 | −1.489 | −15.027 | −4.430 | +0.00 | +0.41 | +0.393 | 2.484 |
| ATOM | 10 | C9 | URI | 1 | −1.811 | −10.567 | −4.533 | −0.42 | +0.12 | +0.174 | 2.484 |
| ATOM | 11 | O14 | URI | 1 | −1.310 | −9.693 | −3.528 | +0.06 | −0.12 | −0.227 | 2.484 |
| ATOM | 12 | C13 | URI | 1 | −1.617 | −8.320 | −3.891 | −0.27 | +0.05 | +0.113 | 2.484 |
| ATOM | 13 | C16 | URI | 1 | −2.105 | −7.584 | −2.643 | −0.18 | +0.04 | +0.113 | 2.484 |
| ATOM | 14 | O17 | URI | 1 | −2.920 | −6.507 | −3.065 | +0.12 | −0.10 | −0.368 | 2.484 |
| ATOM | 15 | P18 | URI | 1 | −2.621 | −5.150 | −3.812 | −0.40 | +0.20 | +1.210 | 2.484 |
| ATOM | 16 | O19 | URI | 1 | −2.765 | −5.393 | −5.275 | +0.02 | −0.07 | −0.850 | 2.484 |
| ATOM | 17 | O20 | URI | 1 | −3.452 | −4.072 | −3.188 | −0.07 | +0.02 | −0.850 | 2.484 |
| ATOM | 18 | O21 | URI | 1 | −1.076 | −5.095 | −3.422 | +0.09 | −0.17 | −0.510 | 2.484 |
| ATOM | 19 | P22 | URI | 1 | −0.016 | −3.902 | −3.318 | −0.18 | +1.15 | +1.210 | 2.484 |
| ATOM | 20 | O25 | URI | 1 | −0.610 | −2.577 | −3.007 | −0.29 | −1.60 | −0.850 | 2.484 |
| ATOM | 21 | O24 | URI | 1 | 0.994 | −4.501 | −2.410 | +0.16 | −0.45 | −0.850 | 2.484 |
| ATOM | 22 | O23 | URI | 1 | 0.560 | −3.734 | −4.817 | −0.24 | −0.42 | −0.368 | 2.484 |
| ATOM | 23 | C37 | URI | 1 | 1.681 | −4.588 | −4.789 | −0.31 | +0.15 | +0.227 | 2.484 |
| ATOM | 24 | C45 | URI | 1 | 2.977 | −3.829 | −5.211 | −0.42 | +0.20 | +0.211 | 2.484 |
| ATOM | 25 | N41 | URI | 1 | 3.132 | −2.610 | −4.370 | −0.28 | −0.65 | −0.650 | 2.484 |
| ATOM | 26 | H41 | URI | 1 | 3.828 | −2.684 | −3.628 | +0.09 | +0.43 | +0.440 | 2.484 |
| ATOM | 27 | C49 | URI | 1 | 2.487 | −1.463 | −4.607 | −0.62 | +0.46 | +0.396 | 2.484 |
| ATOM | 28 | O38 | URI | 1 | 1.653 | −1.315 | −5.502 | −0.59 | −0.70 | −0.396 | 2.484 |
| ATOM | 29 | C50 | URI | 1 | 2.845 | −0.301 | −3.686 | −0.65 | +0.00 | +0.000 | 2.484 |
| ATOM | 30 | C44 | URI | 1 | 2.946 | −3.514 | −6.739 | −0.53 | +0.12 | +0.113 | 2.484 |
| ATOM | 31 | O47 | URI | 1 | 4.203 | −2.992 | −7.161 | −0.27 | −0.98 | −0.537 | 2.484 |
| ATOM | 32 | H47 | URI | 1 | 4.052 | −2.078 | −7.482 | +0.14 | +0.69 | +0.424 | 2.484 |
| ATOM | 33 | C43 | URI | 1 | 2.627 | −4.764 | −7.597 | −0.56 | +0.08 | +0.113 | 2.484 |
| ATOM | 34 | O48 | URI | 1 | 2.469 | −4.363 | −8.953 | −0.13 | −0.38 | −0.537 | 2.484 |
| ATOM | 35 | H48 | URI | 1 | 2.240 | −5.186 | −9.429 | +0.05 | +0.28 | +0.424 | 2.484 |
| ATOM | 36 | C46 | URI | 1 | 1.341 | −5.472 | −7.087 | −0.34 | +0.03 | +0.113 | 2.484 |
| ATOM | 37 | C42 | URI | 1 | 1.054 | −6.804 | −7.797 | −0.60 | +0.04 | +0.113 | 2.484 |
| ATOM | 38 | O39 | URI | 1 | −0.195 | −6.728 | −8.475 | −0.10 | −0.10 | −0.537 | 2.484 |
| ATOM | 39 | H39 | URI | 1 | −0.272 | −5.827 | −8.864 | −0.10 | −0.02 | +0.424 | 2.484 |
| ATOM | 40 | O40 | URI | 1 | 1.462 | −5.739 | −5.658 | +0.08 | −0.09 | −0.227 | 2.484 |
| ATOM | 41 | C12 | URI | 1 | −2.680 | −8.415 | −4.984 | −0.35 | +0.08 | +0.113 | 2.484 |
| ATOM | 42 | O15 | URI | 1 | −2.076 | −8.201 | −6.264 | −0.06 | −0.33 | −0.537 | 2.484 |
| ATOM | 43 | H15 | URI | 1 | −1.420 | −7.494 | −6.122 | +0.09 | +0.15 | +0.424 | 2.484 |
| ATOM | 44 | C10 | URI | 1 | −3.126 | −9.894 | −4.936 | −0.40 | +0.09 | +0.113 | 2.484 |
| ATOM | 45 | O11 | URI | 1 | −3.525 | −10.318 | −6.219 | −0.24 | −0.56 | −0.537 | 2.484 |
| ATOM | 46 | H11 | URI | 1 | −4.324 | −9.786 | −6.414 | +0.06 | +0.44 | +0.424 | 2.484 |
| TER | | | | | | | | | | | |
| ENDMDL | | | | | | | | | | | |
| MODEL | 14 | | | | | | | | | | |
| USER | Run = 14 | | | | | | | | | | |
| USER | Cluster Rank = 18 | | | | | | | | | | |
| USER | Number of conformations in this cluster = 1 | | | | | | | | | | |
| USER | | | | | | | | | | | |
| USER | RMSD from reference structure = 2.557 A | | | | | | | | | | |
| USER | | | | | | | | | | | |
| USER | Estimated Free Energy of Binding = −6.70 kcal/mol [= (1) + (3)] | | | | | | | | | | |
| USER | Estimated Inhibition Constant, Ki = +1.23e−05 [Temperature = 298.15 K] | | | | | | | | | | |
| USER | | | | | | | | | | | |
| USER | Final Docked Energy = −8.60 kcal/mol [= (1) + (2)] | | | | | | | | | | |
| USER | | | | | | | | | | | |
| USER | (1) Final Intermolecular Energy = −11.37 kcal/mol | | | | | | | | | | |
| USER | (2) Final Internal Energy of Ligand = +2.77 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 4.431529 −6.318014 −3.201728 | | | | | | | | | | |
| USER | NEWDPF quat0 0.521421 0.539137 0.661401 −89.209601 | | | | | | | | | | |
| USER | NEWDPF ndihe 15 | | | | | | | | | | |
| USER | NEWDPF dihe0 −52.90 20.66 −22.37 −37.72 −142.56 −47.19 12.68 3.01 −8.22 | | | | | | | | | | |
| | −103.33 −13.14 165.90 −35.48 −35.05 −16.41 | | | | | | | | | | |
| USER | | | | | | | | | | | |
| USER | | | | | x | y | z | vdW | Elec | q | RMS |
| ATOM | 1 | C2 | URI | 1 | −3.332 | −9.104 | −4.868 | −0.38 | +0.24 | +0.296 | 2.557 |
| ATOM | 2 | N3 | URI | 1 | −4.566 | −8.909 | −4.301 | −0.26 | −0.31 | −0.292 | 2.557 |
| ATOM | 3 | C4 | URI | 1 | −4.959 | −7.809 | −3.536 | −0.31 | +0.29 | +0.346 | 2.557 |
| ATOM | 4 | C5 | URI | 1 | −3.940 | −6.826 | −3.343 | −0.32 | +0.00 | +0.000 | 2.557 |
| ATOM | 5 | C6 | URI | 1 | −2.732 | −7.005 | −3.897 | −0.28 | −0.02 | −0.050 | 2.557 |
| ATOM | 6 | N1 | URI | 1 | −2.399 | −8.131 | −4.642 | −0.18 | +0.02 | +0.039 | 2.557 |
| ATOM | 7 | O8 | URI | 1 | −6.107 | −7.791 | −3.092 | −0.04 | −0.39 | −0.396 | 2.557 |
| ATOM | 8 | O7 | URI | 1 | −3.077 | −10.108 | −5.534 | −0.16 | −0.36 | −0.396 | 2.557 |
| ATOM | 9 | H3 | URI | 1 | −5.252 | −9.627 | −4.451 | +0.07 | +0.56 | +0.393 | 2.557 |
| ATOM | 10 | C9 | URI | 1 | −1.053 | −8.284 | −5.207 | −0.36 | +0.08 | +0.174 | 2.557 |
| ATOM | 11 | O14 | URI | 1 | −0.142 | −7.929 | −4.173 | +0.07 | −0.09 | −0.227 | 2.557 |
| ATOM | 12 | C13 | URI | 1 | 1.129 | −7.547 | −4.766 | −0.22 | +0.05 | +0.113 | 2.557 |
| ATOM | 13 | C16 | URI | 1 | 1.643 | −6.297 | −4.050 | −0.34 | +0.06 | +0.113 | 2.557 |
| ATOM | 14 | O17 | URI | 1 | 3.050 | −6.407 | −3.942 | −0.01 | −0.27 | −0.368 | 2.557 |
| ATOM | 15 | P18 | URI | 1 | 4.172 | −5.447 | −3.388 | −0.59 | +1.41 | +1.210 | 2.557 |
| ATOM | 16 | O19 | URI | 1 | 3.703 | −4.904 | −2.081 | −0.05 | −0.84 | −0.850 | 2.557 |
| ATOM | 17 | O20 | URI | 1 | 5.480 | −6.176 | −3.423 | +0.05 | −1.56 | −0.850 | 2.557 |
| ATOM | 18 | O21 | URI | 1 | 4.007 | −4.341 | −4.524 | −0.03 | −0.58 | −0.510 | 2.557 |
| ATOM | 19 | P22 | URI | 1 | 4.039 | −2.743 | −4.507 | −0.49 | +1.40 | +1.210 | 2.557 |
| ATOM | 20 | O25 | URI | 1 | 5.386 | −2.156 | −4.296 | −0.01 | −1.33 | −0.850 | 2.557 |
| ATOM | 21 | O24 | URI | 1 | 3.273 | −2.433 | −5.740 | −0.05 | −1.01 | −0.850 | 2.557 |
| ATOM | 22 | O23 | URI | 1 | 3.217 | −2.331 | −3.179 | +0.16 | −0.28 | −0.368 | 2.557 |
| ATOM | 23 | C37 | URI | 1 | 2.167 | −1.562 | −3.722 | −0.50 | +0.23 | +0.227 | 2.557 |
| ATOM | 24 | C45 | URI | 1 | 1.554 | −0.610 | −2.647 | −0.44 | +0.13 | +0.211 | 2.557 |
| ATOM | 25 | N41 | URI | 1 | 2.638 | 0.205 | −2.033 | −0.34 | −0.07 | −0.650 | 2.557 |
| ATOM | 26 | H41 | URI | 1 | 3.585 | −0.104 | −2.250 | −0.20 | −0.02 | +0.440 | 2.557 |
| ATOM | 27 | C49 | URI | 1 | 2.412 | 1.317 | −1.326 | −0.53 | −0.03 | +0.396 | 2.557 |
| ATOM | 28 | O38 | URI | 1 | 1.289 | 1.746 | −1.055 | −0.03 | +0.05 | −0.396 | 2.557 |
| ATOM | 29 | C50 | URI | 1 | 3.657 | 2.063 | −0.853 | −0.44 | +0.00 | +0.000 | 2.557 |
| ATOM | 30 | C44 | URI | 1 | 0.742 | −1.432 | −1.598 | −0.28 | +0.05 | +0.113 | 2.557 |
| ATOM | 31 | O47 | URI | 1 | 0.029 | −0.555 | −0.730 | +0.10 | −0.02 | −0.537 | 2.557 |
| ATOM | 32 | H47 | URI | 1 | 0.689 | −0.075 | −0.186 | +0.11 | −0.03 | +0.424 | 2.557 |
| ATOM | 33 | C43 | URI | 1 | −0.277 | −2.396 | −2.258 | −0.30 | +0.10 | +0.113 | 2.557 |
| ATOM | 34 | O48 | URI | 1 | −0.862 | −3.208 | −1.247 | +0.13 | −0.11 | −0.537 | 2.557 |
| ATOM | 35 | H48 | URI | 1 | −1.341 | −2.579 | −0.670 | +0.08 | −0.03 | +0.424 | 2.557 |
| ATOM | 36 | C46 | URI | 1 | −0.414 | −3.281 | −3.333 | −0.09 | +0.16 | +0.113 | 2.557 |
| ATOM | 37 | C42 | URI | 1 | −0.568 | −4.151 | −4.132 | −0.17 | +0.09 | +0.113 | 2.557 |
| ATOM | 38 | O39 | URI | 1 | −1.904 | −3.748 | −3.852 | −0.21 | −0.27 | −0.537 | 2.557 |
| ATOM | 39 | H39 | URI | 1 | −2.471 | −4.040 | −4.602 | −0.06 | −0.03 | +0.424 | 2.557 |
| ATOM | 40 | O40 | URI | 1 | 1.136 | −2.433 | −4.275 | −0.30 | −0.47 | −0.227 | 2.557 |
| ATOM | 41 | C12 | URI | 1 | 0.834 | −7.310 | −6.246 | −0.47 | +0.04 | +0.113 | 2.557 |
| ATOM | 42 | O15 | URI | 1 | 1.267 | −8.439 | −7.011 | −0.10 | −0.27 | −0.537 | 2.557 |
| ATOM | 43 | H15 | URI | 1 | 1.943 | −8.091 | −7.621 | +0.11 | +0.25 | +0.424 | 2.557 |
| ATOM | 44 | C10 | URI | 1 | −0.710 | −7.284 | −6.314 | −0.40 | +0.03 | +0.113 | 2.557 |
| ATOM | 45 | O11 | URI | 1 | −1.145 | −7.764 | −7.566 | −0.04 | −0.20 | −0.537 | 2.557 |
| ATOM | 46 | H11 | URI | 1 | −1.618 | −7.005 | −7.967 | −0.00 | +0.09 | +0.424 | 2.557 |
| TER | | | | | | | | | | | |
| ENDMDL | | | | | | | | | | | |
| MODEL | 1 | | | | | | | | | | |
| USER | Run = 1 | | | | | | | | | | |
| USER | Cluster Rank = 19 | | | | | | | | | | |
| USER | Number of conformations in this cluster = 1 | | | | | | | | | | |
| USER | | | | | | | | | | | |
| USER | RMSD from reference structure = 4.128 A | | | | | | | | | | |
| USER | | | | | | | | | | | |
| USER | Estimated Free Energy of Binding = −5.80 kcal/mol [= (1) + (3)] | | | | | | | | | | |
| USER | Estimated Inhibition Constant, Ki = +5.59e−05 [Temperature = 298.15 K] | | | | | | | | | | |
| USER | | | | | | | | | | | |
| USER | Final Docked Energy = −8.58 kcal/mol [= (1) + (2)] | | | | | | | | | | |
| USER | | | | | | | | | | | |
| USER | (1) Final Intermolecular Energy = −10.47 kcal/mol | | | | | | | | | | |
| USER | (2) Final Internal Energy of Ligand = +1.90 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −2.895035 −10.943470 −4.306050 | | | | | | | | | | |
| USER | NEWDPF quat0 −0.741392 0.539450 0.399163 −165.493708 | | | | | | | | | | |
| USER | NEWDPF ndihe 15 | | | | | | | | | | |
| USER | NEWDPF dihe0 39.64 −129.63 −180.00 19.15 151.11 148.22 −19.49 2.51 2.54 | | | | | | | | | | |
| | 179.98 67.99 −120.45 −119.11 −139.40 −15.68 | | | | | | | | | | |
| USER | | | | | | | | | | | |
| USER | | | | | x | y | z | vdW | Elec | q | RMS |
| ATOM | 1 | C2 | URI | 1 | 3.368 | −5.939 | −6.863 | −0.54 | +0.25 | +0.296 | 4.128 |
| ATOM | 2 | N3 | URI | 1 | 3.719 | −5.374 | −8.063 | −0.45 | −0.30 | −0.292 | 4.128 |
| ATOM | 3 | C4 | URI | 1 | 2.861 | −5.103 | −9.130 | −0.75 | +0.27 | +0.346 | 4.128 |
| ATOM | 4 | C5 | URI | 1 | 1.500 | −5.479 | −8.909 | −0.70 | +0.00 | +0.000 | 4.128 |
| ATOM | 5 | C6 | URI | 1 | 1.147 | −6.027 | −7.737 | −0.52 | −0.01 | −0.050 | 4.128 |
| ATOM | 6 | N1 | URI | 1 | 2.054 | −6.283 | −6.714 | −0.28 | +0.02 | +0.039 | 4.128 |
| ATOM | 7 | O8 | URI | 1 | 3.339 | −4.597 | −10.146 | −0.17 | −0.32 | −0.396 | 4.128 |
| ATOM | 8 | O7 | URI | 1 | 4.197 | −6.137 | −5.974 | −0.08 | −0.43 | −0.396 | 4.128 |
| ATOM | 9 | H3 | URI | 1 | 4.686 | −5.134 | −8.185 | +0.02 | +0.58 | +0.393 | 4.128 |
| ATOM | 10 | C9 | URI | 1 | 1.613 | −6.905 | −5.460 | −0.44 | +0.08 | +0.174 | 4.128 |
| ATOM | 11 | O14 | URI | 1 | 0.781 | −7.999 | −5.827 | −0.02 | −0.10 | −0.227 | 4.128 |
| ATOM | 12 | C13 | URI | 1 | −0.093 | −8.330 | −4.714 | −0.13 | +0.05 | +0.113 | 4.128 |
| ATOM | 13 | C16 | URI | 1 | −1.498 | −8.588 | −5.259 | −0.36 | +0.06 | +0.113 | 4.128 |
| ATOM | 14 | O17 | URI | 1 | −1.367 | −9.282 | −6.485 | +0.01 | −0.21 | −0.368 | 4.128 |
| ATOM | 15 | P18 | URI | 1 | −1.356 | −10.812 | −6.869 | −0.70 | +0.89 | +1.210 | 4.128 |
| ATOM | 16 | O19 | URI | 1 | −2.499 | −11.058 | −7.794 | −0.21 | −0.52 | −0.850 | 4.128 |
| ATOM | 17 | O20 | URI | 1 | 0.021 | −11.176 | −7.331 | −0.44 | −1.09 | −0.850 | 4.128 |
| ATOM | 18 | O21 | URI | 1 | −1.719 | −11.342 | −5.409 | −0.18 | −0.42 | −0.510 | 4.128 |
| ATOM | 19 | P22 | URI | 1 | −1.190 | −12.566 | −4.527 | −0.67 | +0.86 | +1.210 | 4.128 |
| ATOM | 20 | O25 | URI | 1 | −0.227 | −13.455 | −5.224 | −0.10 | −0.69 | −0.850 | 4.128 |
| ATOM | 21 | O24 | URI | 1 | −0.794 | −11.864 | −3.281 | −0.06 | −0.55 | −0.850 | 4.128 |
| ATOM | 22 | O23 | URI | 1 | −2.490 | −13.497 | −4.297 | +0.17 | −0.32 | −0.368 | 4.128 |
| ATOM | 23 | C37 | URI | 1 | −2.746 | −13.292 | −2.926 | −0.39 | +0.15 | +0.227 | 4.128 |
| ATOM | 24 | C45 | URI | 1 | −3.600 | −14.456 | −2.334 | −0.28 | +0.12 | +0.211 | 4.128 |
| ATOM | 25 | N41 | URI | 1 | −2.937 | −15.756 | −2.631 | −0.22 | −0.39 | −0.650 | 4.128 |
| ATOM | 26 | H41 | URI | 1 | −2.585 | −15.852 | −3.583 | +0.08 | +0.33 | +0.440 | 4.128 |
| ATOM | 27 | C49 | URI | 1 | −2.725 | −16.702 | −1.710 | −0.31 | +0.20 | +0.396 | 4.128 |
| ATOM | 28 | O38 | URI | 1 | −3.110 | −16.626 | −0.543 | +0.10 | −0.15 | −0.396 | 4.128 |
| ATOM | 29 | C50 | URI | 1 | −1.951 | −17.927 | −2.189 | −0.44 | +0.00 | +0.000 | 4.128 |
| ATOM | 30 | C44 | URI | 1 | −5.063 | −14.383 | −2.872 | −0.22 | +0.06 | +0.113 | 4.128 |
| ATOM | 31 | O47 | URI | 1 | −5.888 | −15.325 | −2.192 | +0.18 | −0.19 | −0.537 | 4.128 |
| ATOM | 32 | H47 | URI | 1 | −5.479 | −15.499 | −1.318 | +0.12 | +0.13 | +0.424 | 4.128 |
| ATOM | 33 | C43 | URI | 1 | −5.690 | −12.976 | −2.700 | −0.26 | +0.07 | +0.113 | 4.128 |
| ATOM | 34 | O48 | URI | 1 | −6.956 | −12.954 | −3.348 | −0.05 | −0.43 | −0.537 | 4.128 |
| ATOM | 35 | H48 | URI | 1 | −6.757 | −12.715 | −4.275 | +0.05 | +0.38 | +0.424 | 4.128 |
| ATOM | 36 | C46 | URI | 1 | −4.759 | −11.879 | −3.289 | −0.26 | +0.09 | +0.113 | 4.128 |
| ATOM | 37 | C42 | URI | 1 | −5.250 | −10.449 | −3.014 | −0.22 | +0.10 | +0.113 | 4.128 |
| ATOM | 38 | O39 | URI | 1 | −4.758 | −9.571 | −4.021 | −0.01 | −0.57 | −0.537 | 4.128 |
| ATOM | 39 | H39 | URI | 1 | −4.220 | −10.104 | −4.650 | +0.08 | +0.43 | +0.424 | 4.128 |
| ATOM | 40 | O40 | URI | 1 | −3.421 | −12.015 | −2.723 | +0.11 | −0.14 | −0.227 | 4.128 |
| ATOM | 41 | C12 | URI | 1 | −0.028 | −7.128 | −3.773 | −0.28 | +0.04 | +0.113 | 4.128 |
| ATOM | 42 | O15 | URI | 1 | 0.835 | −7.426 | −2.670 | +0.02 | −0.18 | −0.537 | 4.128 |
| ATOM | 43 | H15 | URI | 1 | 0.415 | −8.179 | −2.214 | +0.06 | +0.12 | +0.424 | 4.128 |
| ATOM | 44 | C10 | URI | 1 | 0.676 | −6.039 | −4.615 | −0.29 | +0.04 | +0.113 | 4.128 |
| ATOM | 45 | O11 | URI | 1 | 1.415 | −5.184 | −3.774 | +0.13 | −0.30 | −0.537 | 4.128 |
| ATOM | 46 | H11 | URI | 1 | 1.879 | −4.578 | −4.389 | +0.11 | +0.30 | +0.424 | 4.128 |
| TER | | | | | | | | | | | |
| ENDMDL | | | | | | | | | | | |
| MODEL | 23 | | | | | | | | | | |
| USER | Run = 23 | | | | | | | | | | |
| USER | Cluster Rank = 20 | | | | | | | | | | |
| USER | Number of conformations in this cluster = 1 | | | | | | | | | | |
| USER | | | | | | | | | | | |
| USER | RMSD from reference structure = 6.003 A | | | | | | | | | | |
| USER | | | | | | | | | | | |
| USER | Estimated Free Energy of Binding = −5.16 kcal/mol [= (1) + (3)] | | | | | | | | | | |
| USER | Estimated Inhibition Constant, Ki = +1.65e−04 [Temperature = 298.15 K] | | | | | | | | | | |
| USER | | | | | | | | | | | |
| USER | Final Docked Energy = −8.47 kcal/mol [= (1) + (2)] | | | | | | | | | | |
| USER | | | | | | | | | | | |
| USER | (1) Final Intermolecular Energy = −9.83 kcal/mol | | | | | | | | | | |
| USER | (2) Final Internal Energy of Ligand = +1.36 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −1.800622 −3.744887 −3.813789 | | | | | | | | | | |
| USER | NEWDPF quat0 −0.759842 −0.527953 0.379350 122.770807 | | | | | | | | | | |
| USER | NEWDPF ndihe 15 | | | | | | | | | | |
| USER | NEWDPF dihe0 −87.69 −94.64 128.31 −36.84 −173.45 −174.34 8.08 43.68 −5.64 | | | | | | | | | | |
| | −167.44 139.06 −177.78 −89.17 −87.11 −26.44 | | | | | | | | | | |
| USER | | | | | | | | | | | |
| USER | | | | | x | y | z | vdW | Elec | q | RMS |
| ATOM | 1 | C2 | URI | 1 | −3.602 | −5.829 | 4.138 | −0.18 | −0.01 | +0.296 | 6.003 |
| ATOM | 2 | N3 | URI | 1 | −4.526 | −5.404 | 5.057 | −0.17 | −0.02 | −0.292 | 6.003 |
| ATOM | 3 | C4 | URI | 1 | −5.753 | −4.799 | 4.776 | −0.31 | +0.07 | +0.346 | 6.003 |
| ATOM | 4 | C5 | URI | 1 | −6.019 | −4.615 | 3.384 | −0.59 | +0.00 | +0.000 | 6.003 |
| ATOM | 5 | C6 | URI | 1 | −5.123 | −5.032 | 2.477 | −0.38 | +0.01 | −0.050 | 6.003 |
| ATOM | 6 | N1 | URI | 1 | −3.911 | −5.620 | 2.823 | −0.10 | +0.00 | +0.039 | 6.003 |
| ATOM | 7 | O8 | URI | 1 | −6.468 | −4.469 | 5.722 | −0.63 | −0.33 | −0.396 | 6.003 |
| ATOM | 8 | O7 | URI | 1 | −2.544 | −6.359 | 4.480 | +0.20 | −0.01 | −0.396 | 6.003 |
| ATOM | 9 | H3 | URI | 1 | −4.298 | −5.540 | 6.026 | +0.09 | +0.03 | +0.393 | 6.003 |
| ATOM | 10 | C9 | URI | 1 | −2.958 | −6.037 | 1.787 | −0.10 | −0.01 | +0.174 | 6.003 |
| ATOM | 11 | O14 | URI | 1 | −3.063 | −5.081 | 0.739 | +0.15 | +0.03 | −0.227 | 6.003 |
| ATOM | 12 | C13 | URI | 1 | −2.581 | −5.672 | −0.498 | −0.14 | +0.00 | +0.113 | 6.003 |
| ATOM | 13 | C16 | URI | 1 | −3.538 | −5.286 | −1.627 | −0.27 | +0.00 | +0.113 | 6.003 |
| ATOM | 14 | O17 | URI | 1 | −3.065 | −4.083 | −2.203 | +0.05 | +0.05 | −0.368 | 6.003 |
| ATOM | 15 | P18 | URI | 1 | −2.646 | −3.676 | −3.669 | −0.30 | +0.23 | +1.210 | 6.003 |
| ATOM | 16 | O19 | URI | 1 | −3.843 | −3.828 | −4.544 | −0.15 | −0.05 | −0.850 | 6.003 |
| ATOM | 17 | O20 | URI | 1 | −1.974 | −2.338 | −3.615 | −0.31 | −1.10 | −0.850 | 6.003 |
| ATOM | 18 | O21 | URI | 1 | −1.652 | −4.901 | −3.894 | +0.03 | −0.12 | −0.510 | 6.003 |
| ATOM | 19 | P22 | URI | 1 | −0.249 | −5.306 | −3.241 | −0.21 | +0.47 | +1.210 | 6.003 |
| ATOM | 20 | O25 | URI | 1 | 0.157 | −4.465 | −2.086 | +0.17 | −0.36 | −0.850 | 6.003 |
| ATOM | 21 | O24 | URI | 1 | −0.429 | −6.770 | −3.074 | +0.13 | −0.27 | −0.850 | 6.003 |
| ATOM | 22 | O23 | URI | 1 | 0.845 | −4.968 | −4.379 | +0.09 | −0.19 | −0.368 | 6.003 |
| ATOM | 23 | C37 | URI | 1 | 1.386 | −6.243 | −4.641 | −0.34 | +0.10 | +0.227 | 6.003 |
| ATOM | 24 | C45 | URI | 1 | 2.906 | −6.292 | −4.292 | −0.47 | +0.15 | +0.211 | 6.003 |
| ATOM | 25 | N41 | URI | 1 | 3.106 | −5.834 | −2.890 | −0.30 | −0.50 | −0.650 | 6.003 |
| ATOM | 26 | H41 | URI | 1 | 2.319 | −5.328 | −2.486 | +0.10 | +0.26 | +0.440 | 6.003 |
| ATOM | 27 | C49 | URI | 1 | 4.189 | −6.133 | −2.165 | −0.63 | +0.48 | +0.396 | 6.003 |
| ATOM | 28 | O38 | URI | 1 | 5.157 | −6.760 | −2.598 | −0.50 | −0.76 | −0.396 | 6.003 |
| ATOM | 29 | C50 | URI | 1 | 4.170 | −5.646 | −0.719 | −0.61 | +0.00 | +0.000 | 6.003 |
| ATOM | 30 | C44 | URI | 1 | 3.725 | −5.460 | −5.327 | −0.53 | +0.11 | +0.113 | 6.003 |
| ATOM | 31 | O47 | URI | 1 | 5.122 | −5.656 | −5.124 | −0.07 | −0.82 | −0.537 | 6.003 |
| ATOM | 32 | H47 | URI | 1 | 5.467 | −4.867 | −4.655 | +0.02 | +0.73 | +0.424 | 6.003 |
| ATOM | 33 | C43 | URI | 1 | 3.390 | −5.838 | −6.793 | −0.54 | +0.10 | +0.113 | 6.003 |
| ATOM | 34 | O48 | URI | 1 | 4.062 | −4.937 | −7.665 | −0.09 | −0.69 | −0.537 | 6.003 |
| ATOM | 35 | H48 | URI | 1 | 3.816 | −4.048 | −7.340 | +0.07 | +0.58 | +0.424 | 6.003 |
| ATOM | 36 | C46 | URI | 1 | 1.857 | −5.792 | −7.040 | −0.47 | +0.05 | +0.113 | 6.003 |
| ATOM | 37 | C42 | URI | 1 | 1.446 | −6.305 | −8.429 | −0.65 | +0.05 | +0.113 | 6.003 |
| ATOM | 38 | O39 | URI | 1 | 0.847 | −5.249 | −9.173 | −0.17 | −0.14 | −0.537 | 6.003 |
| ATOM | 39 | H39 | URI | 1 | 0.138 | −4.853 | −8.616 | −0.16 | −0.14 | +0.424 | 6.003 |
| ATOM | 40 | O40 | URI | 1 | 1.174 | −6.605 | −6.038 | +0.04 | −0.08 | −0.227 | 6.003 |
| ATOM | 41 | C12 | URI | 1 | −2.537 | −7.178 | −0.240 | −0.09 | +0.01 | +0.113 | 6.003 |
| ATOM | 42 | O15 | URI | 1 | −1.188 | −7.583 | 0.016 | +0.16 | −0.09 | −0.537 | 6.003 |
| ATOM | 43 | H15 | URI | 1 | −0.969 | −8.210 | −0.699 | +0.10 | +0.09 | +0.424 | 6.003 |
| ATOM | 44 | C10 | URI | 1 | −3.318 | −7.348 | 1.083 | −0.06 | +0.01 | +0.113 | 6.003 |
| ATOM | 45 | O11 | URI | 1 | −2.815 | −8.450 | 1.801 | +0.21 | −0.06 | −0.537 | 6.003 |
| ATOM | 46 | H11 | URI | 1 | −3.590 | −8.799 | 2.288 | +0.12 | +0.05 | +0.424 | 6.003 |
| TER | | | | | | | | | | | |
| ENDMDL | | | | | | | | | | | |
| MODEL | 32 | | | | | | | | | | |
| USER | Run = 32 | | | | | | | | | | |
| USER | Cluster Rank = 21 | | | | | | | | | | |
| USER | Number of conformations in this cluster = 1 | | | | | | | | | | |
| USER | | | | | | | | | | | |
| USER | RMSD from reference structure = 3.573 A | | | | | | | | | | |
| USER | | | | | | | | | | | |
| USER | Estimated Free Energy of Binding = −5.94 kcal/mol [= (1) + (3)] | | | | | | | | | | |
| USER | Estimated Inhibition Constant, Ki = +4.40e−05 [Temperature = 298.15 K] | | | | | | | | | | |
| USER | | | | | | | | | | | |
| USER | Final Docked Energy = −8.37 kcal/mol [= (1) + (2)] | | | | | | | | | | |
| USER | | | | | | | | | | | |
| USER | (1) Final Intermolecular Energy = −10.61 kcal/mol | | | | | | | | | | |
| USER | (2) Final Internal Energy of Ligand = +2.24 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −1.226783 −3.904388 −0.459467 | | | | | | | | | | |
| USER | NEWDPF quat0 −0.586843 −0.659745 −0.469416 −86.980885 | | | | | | | | | | |
| USER | NEWDPF ndihe 15 | | | | | | | | | | |
| USER | NEWDPF dihe0 8.75 −1.27 87.11 −3.68 149.00 107.80 −41.27 24.28 27.06 −154.17 | | | | | | | | | | |
| | 33.47 −151.89 1.73 120.59 32.74 | | | | | | | | | | |
| USER | | | | | | | | | | | |
| USER | | | | | x | y | z | vdW | Elec | q | RMS |
| ATOM | 1 | C2 | URI | 1 | −2.905 | −9.233 | −6.753 | −0.56 | +0.23 | +0.296 | 3.573 |
| ATOM | 2 | N3 | URI | 1 | −2.139 | −9.921 | −7.660 | −0.37 | −0.18 | −0.292 | 3.573 |
| ATOM | 3 | C4 | URI | 1 | −0.801 | −10.287 | −7.503 | −0.71 | +0.25 | +0.346 | 3.573 |
| ATOM | 4 | C5 | URI | 1 | −0.213 | −9.870 | −6.268 | −0.59 | +0.00 | +0.000 | 3.573 |
| ATOM | 5 | C6 | URI | 1 | −0.954 | −9.201 | −5.373 | −0.41 | −0.03 | −0.050 | 3.573 |
| ATOM | 6 | N1 | URI | 1 | −2.283 | −8.857 | −5.595 | −0.22 | +0.03 | +0.039 | 3.573 |
| ATOM | 7 | O8 | URI | 1 | −0.255 | −10.899 | −8.420 | −0.42 | −0.42 | −0.396 | 3.573 |
| ATOM | 8 | O7 | URI | 1 | −4.084 | −8.958 | −6.977 | −0.20 | −0.40 | −0.396 | 3.573 |
| ATOM | 9 | H3 | URI | 1 | −2.585 | −10.187 | −8.520 | +0.05 | +0.21 | +0.393 | 3.573 |
| ATOM | 10 | C9 | URI | 1 | −3.040 | −8.104 | −4.587 | −0.32 | +0.12 | +0.174 | 3.573 |
| ATOM | 11 | O14 | URI | 1 | −2.932 | −6.732 | −4.948 | −0.10 | −0.16 | −0.227 | 3.573 |
| ATOM | 12 | C13 | URI | 1 | −3.179 | −5.908 | −3.777 | −0.37 | +0.03 | +0.113 | 3.573 |
| ATOM | 13 | C16 | URI | 1 | −2.149 | −4.779 | −3.751 | −0.38 | +0.02 | +0.113 | 3.573 |
| ATOM | 14 | O17 | URI | 1 | −0.864 | −5.365 | −3.655 | +0.10 | −0.11 | −0.368 | 3.573 |
| ATOM | 15 | P18 | URI | 1 | 0.478 | −4.925 | −2.952 | −0.19 | +0.60 | +1.210 | 3.573 |
| ATOM | 16 | O19 | URI | 1 | 0.444 | −5.429 | −1.550 | +0.17 | −0.27 | −0.850 | 3.573 |
| ATOM | 17 | O20 | URI | 1 | 1.626 | −5.331 | −3.824 | +0.12 | −0.48 | −0.850 | 3.573 |
| ATOM | 18 | O21 | URI | 1 | 0.160 | −3.362 | −2.972 | −0.16 | −0.57 | −0.510 | 3.573 |
| ATOM | 19 | P22 | URI | 1 | 1.017 | −2.067 | −2.592 | −0.30 | +1.15 | +1.210 | 3.573 |
| ATOM | 20 | O25 | URI | 1 | 2.487 | −2.277 | −2.604 | +0.07 | −0.53 | −0.850 | 3.573 |
| ATOM | 21 | O24 | URI | 1 | 0.399 | −1.051 | −3.481 | −0.23 | −1.78 | −0.850 | 3.573 |
| ATOM | 22 | O23 | URI | 1 | 0.671 | −1.788 | −1.040 | +0.15 | −0.10 | −0.368 | 3.573 |
| ATOM | 23 | C37 | URI | 1 | −0.514 | −1.032 | −1.145 | −0.35 | +0.03 | +0.227 | 3.573 |
| ATOM | 24 | C45 | URI | 1 | −0.500 | 0.176 | −0.157 | −0.36 | −0.04 | +0.211 | 3.573 |
| ATOM | 25 | N41 | URI | 1 | 0.739 | 0.974 | −0.369 | −0.19 | +0.10 | −0.650 | 3.573 |
| ATOM | 26 | H41 | URI | 1 | 1.606 | 0.494 | −0.130 | +0.11 | −0.04 | +0.440 | 3.573 |
| ATOM | 27 | C49 | URI | 1 | 0.745 | 2.193 | −0.917 | −0.54 | −0.09 | +0.396 | 3.573 |
| ATOM | 28 | O38 | URI | 1 | −0.272 | 2.807 | −1.243 | −0.10 | +0.10 | −0.396 | 3.573 |
| ATOM | 29 | C50 | URI | 1 | 2.122 | 2.813 | −1.132 | −0.55 | +0.00 | +0.000 | 3.573 |
| ATOM | 30 | C44 | URI | 1 | −0.657 | −0.326 | 1.313 | −0.31 | −0.06 | +0.113 | 3.573 |
| ATOM | 31 | O47 | URI | 1 | −0.833 | 0.779 | 2.195 | +0.12 | +0.58 | −0.537 | 3.573 |
| ATOM | 32 | H47 | URI | 1 | −0.716 | 1.599 | 1.671 | +0.09 | −0.37 | +0.424 | 3.573 |
| ATOM | 33 | C43 | URI | 1 | −1.861 | −1.285 | 1.488 | −0.45 | −0.08 | +0.113 | 3.573 |
| ATOM | 34 | O48 | URI | 1 | −1.842 | −1.810 | 2.811 | −0.01 | +0.39 | −0.537 | 3.573 |
| ATOM | 35 | H48 | URI | 1 | −1.605 | −1.049 | 3.376 | −0.23 | −0.54 | +0.424 | 3.573 |
| ATOM | 36 | C46 | URI | 1 | −1.809 | −2.436 | 0.446 | −0.31 | −0.04 | +0.113 | 3.573 |
| ATOM | 37 | C42 | URI | 1 | −3.056 | −3.335 | 0.465 | −0.16 | −0.06 | +0.113 | 3.573 |
| ATOM | 38 | O39 | URI | 1 | −3.087 | −4.137 | −0.710 | +0.09 | +0.14 | −0.537 | 3.573 |
| ATOM | 39 | H39 | URI | 1 | −3.621 | −3.661 | −1.386 | −0.19 | −0.23 | +0.424 | 3.573 |
| ATOM | 40 | O40 | URI | 1 | −1.676 | −1.877 | −0.896 | +0.09 | +0.05 | −0.227 | 3.573 |
| ATOM | 41 | C12 | URI | 1 | −3.061 | −6.853 | −2.581 | −0.20 | +0.03 | +0.113 | 3.573 |
| ATOM | 42 | O15 | URI | 1 | −4.367 | −7.209 | −2.117 | +0.13 | −0.15 | −0.537 | 3.573 |
| ATOM | 43 | H15 | URI | 1 | −4.629 | −6.489 | −1.514 | +0.11 | +0.04 | +0.424 | 3.573 |
| ATOM | 44 | C10 | URI | 1 | −2.432 | −8.131 | −3.183 | −0.21 | +0.05 | +0.113 | 3.573 |
| ATOM | 45 | O11 | URI | 1 | −2.873 | −9.264 | −2.473 | +0.16 | −0.23 | −0.537 | 3.573 |
| ATOM | 46 | H11 | URI | 1 | −2.518 | −10.024 | −2.980 | +0.10 | +0.22 | +0.424 | 3.573 |
| TER | | | | | | | | | | | |
| ENDMDL | | | | | | | | | | | |
| MODEL | 8 | | | | | | | | | | |
| USER | Run = 8 | | | | | | | | | | |
| USER | Cluster Rank = 22 | | | | | | | | | | |
| USER | Number of conformations in this cluster = 1 | | | | | | | | | | |
| USER | | | | | | | | | | | |
| USER | RMSD from reference structure = 2.841 A | | | | | | | | | | |
| USER | | | | | | | | | | | |
| USER | Estimated Free Energy of Binding = −6.22 kcal/mol [= (1) + (3)] | | | | | | | | | | |
| USER | Estimated Inhibition Constant, Ki = +2.76e−05 [Temperature =298.15 K] | | | | | | | | | | |
| USER | | | | | | | | | | | |
| USER | Final Docked Energy = −8.27 kcal/mol [= (1) + (2)] | | | | | | | | | | |
| USER | | | | | | | | | | | |
| USER | (1) Final Intermolecular Energy = −10.89 kcal/mol | | | | | | | | | | |
| USER | (2) Final Internal Energy of Ligand = +2.62 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 2.383864 −6.391182 −5.360228 | | | | | | | | | | |
| USER | NEWDPF quat0 0.673976 0.701601 0.231329 −66.198628 | | | | | | | | | | |
| USER | NEWDPF ndihe 15 | | | | | | | | | | |
| USER | NEWDPF dihe0 −95.31 149.55 25.88 −87.16 174.85 −136.14 4.97 −20.89 9.95 | | | | | | | | | | |
| | −116.13 −173.41 100.90 −142.48 −49.38 −42.44 | | | | | | | | | | |
| USER | | | | | | | | | | | |
| USER | | | | | x | y | z | vdW | Elec | q | RMS |
| ATOM | 1 | C2 | URI | 1 | −2.336 | −13.202 | −3.892 | −0.39 | +0.23 | +0.296 | 2.841 |
| ATOM | 2 | N3 | URI | 1 | −3.242 | −13.554 | −2.924 | −0.20 | −0.20 | −0.292 | 2.841 |
| ATOM | 3 | C4 | URI | 1 | −3.988 | −12.677 | −2.134 | −0.22 | +0.18 | +0.346 | 2.841 |
| ATOM | 4 | C5 | URI | 1 | −3.739 | −11.292 | −2.386 | −0.22 | +0.00 | +0.000 | 2.841 |
| ATOM | 5 | C6 | URI | 1 | −2.859 | −10.939 | −3.334 | −0.31 | −0.03 | −0.050 | 2.841 |
| ATOM | 6 | N1 | URI | 1 | −2.137 | −11.863 | −4.082 | −0.29 | +0.03 | +0.039 | 2.841 |
| ATOM | 7 | O8 | URI | 1 | −4.756 | −13.157 | −1.301 | +0.18 | −0.16 | −0.396 | 2.841 |
| ATOM | 8 | O7 | URI | 1 | −1.725 | −14.048 | −4.546 | −0.19 | −0.30 | −0.396 | 2.841 |
| ATOM | 9 | H3 | URI | 1 | −3.381 | −14.536 | −2.769 | +0.10 | +0.25 | +0.393 | 2.841 |
| ATOM | 10 | C9 | URI | 1 | −1.170 | −11.417 | −5.092 | −0.56 | +0.13 | +0.174 | 2.841 |
| ATOM | 11 | O14 | URI | 1 | −0.857 | −10.066 | −4.777 | +0.06 | −0.14 | −0.227 | 2.841 |
| ATOM | 12 | C13 | URI | 1 | −0.351 | −9.402 | −5.967 | −0.50 | +0.07 | +0.113 | 2.841 |
| ATOM | 13 | C16 | URI | 1 | −0.983 | −8.012 | −6.055 | −0.42 | +0.05 | +0.113 | 2.841 |
| ATOM | 14 | O17 | URI | 1 | 0.060 | −7.072 | −6.228 | +0.04 | −0.08 | −0.368 | 2.841 |
| ATOM | 15 | P18 | URI | 1 | 1.046 | −6.755 | −7.419 | −0.54 | +0.39 | +1.210 | 2.841 |
| ATOM | 16 | O19 | URI | 1 | 1.073 | −7.945 | −8.316 | −0.09 | −0.50 | −0.850 | 2.841 |
| ATOM | 17 | O20 | URI | 1 | 0.669 | −5.431 | −8.008 | +0.13 | +0.06 | −0.850 | 2.841 |
| ATOM | 18 | O21 | URI | 1 | 2.366 | −6.723 | −6.524 | −0.01 | −0.29 | −0.510 | 2.841 |
| ATOM | 19 | P22 | URI | 1 | 2.620 | −6.530 | −4.957 | −0.47 | +0.76 | +1.210 | 2.841 |
| ATOM | 20 | O25 | URI | 1 | 1.962 | −7.547 | −4.098 | +0.04 | −0.43 | −0.850 | 2.841 |
| ATOM | 21 | O24 | URI | 1 | 4.095 | −6.370 | −4.930 | −0.10 | −0.85 | −0.850 | 2.841 |
| ATOM | 22 | O23 | URI | 1 | 1.866 | −5.152 | −4.586 | +0.11 | −0.22 | −0.368 | 2.841 |
| ATOM | 23 | C37 | URI | 1 | 2.945 | −4.247 | −4.516 | −0.38 | +0.20 | +0.227 | 2.841 |
| ATOM | 24 | C45 | URI | 1 | 2.447 | −2.799 | −4.216 | −0.40 | +0.21 | +0.211 | 2.841 |
| ATOM | 25 | N41 | URI | 1 | 1.598 | −2.812 | −2.993 | −0.17 | −0.59 | −0.650 | 2.841 |
| ATOM | 26 | H41 | URI | 1 | 1.219 | −3.726 | −2.741 | +0.11 | +0.32 | +0.440 | 2.841 |
| ATOM | 27 | C49 | URI | 1 | 1.408 | −1.741 | −2.216 | −0.31 | +0.25 | +0.396 | 2.841 |
| ATOM | 28 | O38 | URI | 1 | 1.860 | −0.622 | −2.463 | −0.01 | −0.19 | −0.396 | 2.841 |
| ATOM | 29 | C50 | URI | 1 | 0.577 | −1.977 | −0.958 | −0.21 | +0.00 | +0.000 | 2.841 |
| ATOM | 30 | C44 | URI | 1 | 1.712 | −2.211 | −5.461 | −0.54 | +0.16 | +0.113 | 2.841 |
| ATOM | 31 | O47 | URI | 1 | 1.424 | −0.831 | −5.257 | −0.66 | −1.13 | −0.537 | 2.841 |
| ATOM | 32 | H47 | URI | 1 | 0.453 | −0.743 | −5.149 | +0.02 | +1.21 | +0.424 | 2.841 |
| ATOM | 33 | C43 | URI | 1 | 2.544 | −2.347 | −6.761 | −0.52 | +0.13 | +0.113 | 2.841 |
| ATOM | 34 | O48 | URI | 1 | 1.747 | −1.927 | −7.863 | −0.09 | −0.63 | −0.537 | 2.841 |
| ATOM | 35 | H48 | URI | 1 | 2.243 | −2.220 | −8.653 | +0.06 | +0.46 | +0.424 | 2.841 |
| ATOM | 36 | C46 | URI | 1 | 3.022 | −3.811 | −6.963 | −0.54 | +0.11 | +0.113 | 2.841 |
| ATOM | 37 | C42 | URI | 1 | 3.981 | −3.984 | −8.152 | −0.56 | +0.16 | +0.113 | 2.841 |
| ATOM | 38 | O39 | URI | 1 | 3.240 | −3.997 | −9.367 | −0.17 | −0.48 | −0.537 | 2.841 |
| ATOM | 39 | H39 | URI | 1 | 3.760 | −4.504 | −10.032 | −0.07 | +0.35 | +0.424 | 2.841 |
| ATOM | 40 | O40 | URI | 1 | 3.707 | −4.273 | −5.760 | +0.01 | −0.27 | −0.227 | 2.841 |
| ATOM | 41 | C12 | URI | 1 | −0.736 | −10.309 | −7.135 | −0.71 | +0.08 | +0.113 | 2.841 |
| ATOM | 42 | O15 | URI | 1 | 0.405 | −11.066 | −7.553 | −0.42 | −0.70 | −0.537 | 2.841 |
| ATOM | 43 | H15 | URI | 1 | 0.146 | −11.470 | −8.402 | +0.03 | +0.58 | +0.424 | 2.841 |
| ATOM | 44 | C10 | URI | 1 | −1.736 | −11.308 | −6.510 | −0.65 | +0.09 | +0.113 | 2.841 |
| ATOM | 45 | O11 | URI | 1 | −1.654 | −12.549 | −7.173 | +0.29 | −0.22 | −0.537 | 2.841 |
| ATOM | 46 | H11 | URI | 1 | −2.496 | −12.999 | −6.953 | −0.22 | +0.01 | +0.424 | 2.841 |
| TER | | | | | | | | | | | |
| ENDMDL | | | | | | | | | | | |
| MODEL | 41 | | | | | | | | | | |
| USER | Run = 41 | | | | | | | | | | |
| USER | Cluster Rank = 23 | | | | | | | | | | |
| USER | Number of conformations in this cluster = 1 | | | | | | | | | | |
| USER | | | | | | | | | | | |
| USER | RMSD from reference structure = 4.246 A | | | | | | | | | | |
| USER | | | | | | | | | | | |
| USER | Estimated Free Energy of Binding = −6.41 kcal/mol [= (1) + (3)] | | | | | | | | | | |
| USER | Estimated Inhibition Constant, Ki = +2.00e−05 [Temperature = 298.15 K] | | | | | | | | | | |
| USER | | | | | | | | | | | |
| USER | Final Docked Energy = −8.20 kcal/mol [= (1) + (2)] | | | | | | | | | | |
| USER | | | | | | | | | | | |
| USER | (1) Final Intermolecular Energy = −11.08 kcal/mol | | | | | | | | | | |
| USER | (2) Final Internal Energy of Ligand = +2.88 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −0.398767 −2.228535 −0.687848 | | | | | | | | | | |
| USER | NEWDPF quat0 −0.979119 0.091400 −0.181579 −27.327284 | | | | | | | | | | |
| USER | NEWDPF ndihe 15 | | | | | | | | | | |
| USER | NEWDPF dihe0 −153.99 16.76 91.70 −54.46 8.55 154.79 124.82 −11.24 −11.92 | | | | | | | | | | |
| | 137.64 −179.94 59.98 18.73 3.12 38.88 | | | | | | | | | | |
| USER | | | | | | | | | | | |
| USER | | | | | x | y | z | vdW | Elec | q | RMS |
| ATOM | 1 | C2 | URI | 1 | −2.203 | −8.656 | −5.811 | −0.41 | +0.19 | +0.296 | 4.246 |
| ATOM | 2 | N3 | URI | 1 | −2.030 | −10.016 | −5.745 | −0.27 | −0.21 | −0.292 | 4.246 |
| ATOM | 3 | C4 | URI | 1 | −1.907 | −10.777 | −4.582 | −0.44 | +0.25 | +0.346 | 4.246 |
| ATOM | 4 | C5 | URI | 1 | −1.957 | −10.022 | −3.369 | −0.32 | +0.00 | +0.000 | 4.246 |
| ATOM | 5 | C6 | URI | 1 | −2.130 | −8.693 | −3.423 | −0.25 | −0.02 | −0.050 | 4.246 |
| ATOM | 6 | N1 | URI | 1 | −2.237 | −7.989 | −4.618 | −0.17 | +0.02 | +0.039 | 4.246 |
| ATOM | 7 | O8 | URI | 1 | −1.750 | −11.992 | −4.697 | −0.14 | −0.32 | −0.396 | 4.246 |
| ATOM | 8 | O7 | URI | 1 | −2.308 | −8.068 | −6.888 | −0.18 | −0.26 | −0.396 | 4.246 |
| ATOM | 9 | H3 | URI | 1 | −1.989 | −10.510 | −6.619 | +0.08 | +0.29 | +0.393 | 4.246 |
| ATOM | 10 | C9 | URI | 1 | −2.404 | −6.531 | −4.616 | −0.34 | +0.07 | +0.174 | 4.246 |
| ATOM | 11 | O14 | URI | 1 | −1.121 | −5.982 | −4.894 | +0.07 | −0.02 | −0.227 | 4.246 |
| ATOM | 12 | C13 | URI | 1 | −1.070 | −4.614 | −4.405 | +0.00 | +0.03 | +0.113 | 4.246 |
| ATOM | 13 | C16 | URI | 1 | 0.274 | −4.395 | −3.710 | −0.24 | +0.08 | +0.113 | 4.246 |
| ATOM | 14 | O17 | URI | 1 | 0.297 | −3.068 | −3.219 | −0.22 | −0.57 | −0.368 | 4.246 |
| ATOM | 15 | P18 | URI | 1 | 1.333 | −1.885 | −3.342 | −0.30 | +1.59 | +1.210 | 4.246 |
| ATOM | 16 | O19 | URI | 1 | 2.619 | −2.340 | −2.742 | +0.08 | −0.56 | −0.850 | 4.246 |
| ATOM | 17 | O20 | URI | 1 | 1.343 | −1.409 | −4.762 | −0.57 | −1.69 | −0.850 | 4.246 |
| ATOM | 18 | O21 | URI | 1 | 0.579 | −0.910 | −2.330 | −0.01 | −0.37 | −0.510 | 4.246 |
| ATOM | 19 | P22 | URI | 1 | −0.950 | −0.769 | −1.883 | −0.33 | +0.39 | +1.210 | 4.246 |
| ATOM | 20 | O25 | URI | 1 | −1.922 | −0.767 | −3.005 | −0.11 | −0.45 | −0.850 | 4.246 |
| ATOM | 21 | O24 | URI | 1 | −1.030 | −1.808 | −0.825 | +0.07 | +0.01 | −0.850 | 4.246 |
| ATOM | 22 | O23 | URI | 1 | −1.060 | 0.718 | −1.264 | −0.04 | +0.02 | −0.368 | 4.246 |
| ATOM | 23 | C37 | URI | 1 | −1.560 | 0.453 | 0.028 | −0.53 | −0.10 | +0.227 | 4.246 |
| ATOM | 24 | C45 | URI | 1 | −1.931 | 1.774 | 0.770 | −0.63 | −0.15 | +0.211 | 4.246 |
| ATOM | 25 | N41 | URI | 1 | −2.858 | 2.573 | −0.078 | −0.52 | +0.32 | −0.650 | 4.246 |
| ATOM | 26 | H41 | URI | 1 | −3.843 | 2.503 | 0.178 | +0.04 | −0.20 | +0.440 | 4.246 |
| ATOM | 27 | C49 | URI | 1 | −2.446 | 3.400 | −1.045 | −0.80 | −0.12 | +0.396 | 4.246 |
| ATOM | 28 | O38 | URI | 1 | −1.268 | 3.537 | −1.377 | −0.15 | +0.10 | −0.396 | 4.246 |
| ATOM | 29 | C50 | URI | 1 | −3.543 | 4.198 | −1.743 | −0.87 | +0.00 | +0.000 | 4.246 |
| ATOM | 30 | C44 | URI | 1 | −0.638 | 2.556 | 1.162 | −0.48 | −0.09 | +0.113 | 4.246 |
| ATOM | 31 | O47 | URI | 1 | −0.964 | 3.661 | 2.000 | −0.03 | +0.69 | −0.537 | 4.246 |
| ATOM | 32 | H47 | URI | 1 | −0.165 | 4.223 | 2.073 | +0.09 | −0.86 | +0.424 | 4.246 |
| ATOM | 33 | C43 | URI | 1 | 0.390 | 1.668 | 1.907 | −0.32 | −0.08 | +0.113 | 4.246 |
| ATOM | 34 | O48 | URI | 1 | 1.590 | 2.410 | 2.093 | +0.09 | +0.35 | −0.537 | 4.246 |
| ATOM | 35 | H48 | URI | 1 | 1.320 | 3.191 | 2.615 | −0.06 | −0.46 | +0.424 | 4.246 |
| ATOM | 36 | C46 | URI | 1 | 0.675 | 0.363 | 1.112 | −0.21 | −0.04 | +0.113 | 4.246 |
| ATOM | 37 | C42 | URI | 1 | 1.569 | −0.632 | 1.870 | −0.13 | −0.02 | +0.113 | 4.246 |
| ATOM | 38 | O39 | URI | 1 | 2.375 | −1.353 | 0.945 | +0.15 | −0.01 | −0.537 | 4.246 |
| ATOM | 39 | H39 | URI | 1 | 1.867 | −2.145 | 0.656 | +0.11 | +0.04 | +0.424 | 4.246 |
| ATOM | 40 | O40 | URI | 1 | −0.583 | −0.306 | 0.801 | +0.09 | +0.09 | −0.227 | 4.246 |
| ATOM | 41 | C12 | URI | 1 | −2.262 | −4.474 | −3.459 | −0.39 | +0.02 | +0.113 | 4.246 |
| ATOM | 42 | O15 | URI | 1 | −3.325 | −3.780 | −4.122 | −0.15 | −0.03 | −0.537 | 4.246 |
| ATOM | 43 | H15 | URI | 1 | −3.654 | −3.135 | −3.469 | −0.05 | +0.00 | +0.424 | 4.246 |
| ATOM | 44 | C10 | URI | 1 | −2.737 | −5.930 | −3.249 | −0.29 | +0.03 | +0.113 | 4.246 |
| ATOM | 45 | O11 | URI | 1 | −4.128 | −5.955 | −3.023 | +0.04 | −0.10 | −0.537 | 4.246 |
| ATOM | 46 | H11 | URI | 1 | −4.475 | −5.215 | −3.562 | +0.07 | +0.04 | +0.424 | 4.246 |
| TER | | | | | | | | | | | |
| ENDMDL | | | | | | | | | | | |
| MODEL | 21 | | | | | | | | | | |
| USER | Run = 21 | | | | | | | | | | |
| USER | Cluster Rank = 24 | | | | | | | | | | |
| USER | Number of conformations in this cluster = 1 | | | | | | | | | | |
| USER | | | | | | | | | | | |
| USER | RMSD from reference structure = 2.765 A | | | | | | | | | | |
| USER | | | | | | | | | | | |
| USER | Estimated Free Energy of Binding = −5.92 kcal/mol [= (1) + (3)] | | | | | | | | | | |
| USER | Estimated Inhibition Constant, Ki = +4.56e−05 [Temperature = 298.15 K] | | | | | | | | | | |
| USER | | | | | | | | | | | |
| USER | Final Docked Energy = −8.17 kcal/mol [= (1) + (2)] | | | | | | | | | | |
| USER | | | | | | | | | | | |
| USER | (1) Final Intermolecular Energy = −10.59 kcal/mol | | | | | | | | | | |
| USER | (2) Final Internal Energy of Ligand = +2.42 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 0.964891 −3.752031 −4.416769 | | | | | | | | | | |
| USER | NEWDPF quat0 0.123849 0.970112 −0.208674 −6.493818 | | | | | | | | | | |
| USER | NEWDPF ndihe 15 | | | | | | | | | | |
| USER | NEWDPF dihe0 83.98 −153.69 167.36 26.39 53.80 127.71 −100.18 0.03 −54.90 | | | | | | | | | | |
| | −114.03 143.34 −18.16 −98.51 −76.73 −10.42 | | | | | | | | | | |
| USER | | | | | | | | | | | |
| USER | | | | | x | y | z | vdW | Elec | q | RMS |
| ATOM | 1 | C2 | URI | 1 | −1.245 | −11.674 | −6.199 | −0.66 | +0.25 | +0.296 | 2.765 |
| ATOM | 2 | N3 | URI | 1 | −1.215 | −12.859 | −5.508 | −0.42 | −0.21 | −0.292 | 2.765 |
| ATOM | 3 | C4 | URI | 1 | −1.225 | −13.006 | −4.120 | −0.71 | +0.24 | +0.346 | 2.765 |
| ATOM | 4 | C5 | URI | 1 | −1.255 | −11.776 | −3.392 | −0.48 | +0.00 | +0.000 | 2.765 |
| ATOM | 5 | C6 | URI | 1 | −1.288 | −10.613 | −4.059 | −0.27 | −0.03 | −0.050 | 2.765 |
| ATOM | 6 | N1 | URI | 1 | −1.266 | −10.532 | −5.447 | −0.32 | +0.03 | +0.039 | 2.765 |
| ATOM | 7 | O8 | URI | 1 | −1.188 | −14.145 | −3.657 | −0.22 | −0.31 | −0.396 | 2.765 |
| ATOM | 8 | O7 | URI | 1 | −1.239 | −11.641 | −7.430 | −0.27 | −0.30 | −0.396 | 2.765 |
| ATOM | 9 | H3 | URI | 1 | −1.184 | −13.701 | −6.054 | −0.20 | +0.15 | +0.393 | 2.765 |
| ATOM | 10 | C9 | URI | 1 | −1.283 | −9.230 | −6.123 | −0.47 | +0.10 | +0.174 | 2.765 |
| ATOM | 11 | O14 | URI | 1 | −0.246 | −8.458 | −5.529 | +0.03 | −0.10 | −0.227 | 2.765 |
| ATOM | 12 | C13 | URI | 1 | −0.518 | −7.046 | −5.741 | −0.35 | +0.02 | +0.113 | 2.765 |
| ATOM | 13 | C16 | URI | 1 | −0.228 | −6.295 | −4.441 | −0.27 | +0.03 | +0.113 | 2.765 |
| ATOM | 14 | O17 | URI | 1 | 1.036 | −5.671 | −4.573 | +0.10 | −0.16 | −0.368 | 2.765 |
| ATOM | 15 | P18 | URI | 1 | 1.498 | −4.165 | −4.647 | −0.29 | +0.94 | +1.210 | 2.765 |
| ATOM | 16 | O19 | URI | 1 | 2.554 | −4.063 | −5.694 | +0.07 | −0.72 | −0.850 | 2.765 |
| ATOM | 17 | O20 | URI | 1 | 0.282 | −3.301 | −4.781 | −0.20 | −1.75 | −0.850 | 2.765 |
| ATOM | 18 | O21 | URI | 1 | 2.167 | −4.136 | −3.200 | +0.11 | −0.37 | −0.510 | 2.765 |
| ATOM | 19 | P22 | URI | 1 | 1.586 | −4.322 | −1.722 | −0.20 | +0.58 | +1.210 | 2.765 |
| ATOM | 20 | O25 | URI | 1 | 0.967 | −5.647 | −1.467 | +0.14 | −0.29 | −0.850 | 2.765 |
| ATOM | 21 | O24 | URI | 1 | 2.739 | −3.865 | −0.907 | +0.12 | −0.43 | −0.850 | 2.765 |
| ATOM | 22 | O23 | URI | 1 | 0.349 | −3.286 | −1.635 | +0.16 | −0.17 | −0.368 | 2.765 |
| ATOM | 23 | C37 | URI | 1 | 0.091 | −3.268 | −0.249 | −0.13 | +0.03 | +0.227 | 2.765 |
| ATOM | 24 | C45 | URI | 1 | −1.337 | −2.716 | 0.049 | −0.25 | −0.03 | +0.211 | 2.765 |
| ATOM | 25 | N41 | URI | 1 | −2.339 | −3.489 | −0.735 | −0.11 | +0.16 | −0.650 | 2.765 |
| ATOM | 26 | H41 | URI | 1 | −2.948 | −2.926 | −1.330 | −0.19 | −0.35 | +0.440 | 2.765 |
| ATOM | 27 | C49 | URI | 1 | −2.396 | −4.825 | −0.746 | −0.19 | −0.01 | +0.396 | 2.765 |
| ATOM | 28 | O38 | URI | 1 | −1.671 | −5.551 | −0.064 | +0.20 | −0.02 | −0.396 | 2.765 |
| ATOM | 29 | C50 | URI | 1 | −3.436 | −5.434 | −1.682 | −0.25 | +0.00 | +0.000 | 2.765 |
| ATOM | 30 | C44 | URI | 1 | −1.395 | −1.183 | −0.233 | −0.38 | −0.03 | +0.113 | 2.765 |
| ATOM | 31 | O47 | URI | 1 | −2.636 | −0.644 | 0.215 | −0.03 | +0.37 | −0.537 | 2.765 |
| ATOM | 32 | H47 | URI | 1 | −2.636 | −0.676 | 1.196 | +0.02 | −0.39 | +0.424 | 2.765 |
| ATOM | 33 | C43 | URI | 1 | −0.249 | −0.406 | 0.464 | −0.27 | −0.03 | +0.113 | 2.765 |
| ATOM | 34 | O48 | URI | 1 | −0.286 | 0.950 | 0.036 | +0.07 | +0.16 | −0.537 | 2.765 |
| ATOM | 35 | H48 | URI | 1 | 0.517 | 1.059 | −0.511 | +0.10 | −0.06 | +0.424 | 2.765 |
| ATOM | 36 | C46 | URI | 1 | 1.130 | −1.042 | 0.136 | −0.19 | +0.00 | +0.113 | 2.765 |
| ATOM | 37 | C42 | URI | 1 | 2.295 | −0.415 | 0.918 | −0.19 | −0.01 | +0.113 | 2.765 |
| ATOM | 38 | O39 | URI | 1 | 3.528 | −0.964 | 0.467 | +0.18 | +0.05 | −0.537 | 2.765 |
| ATOM | 39 | H39 | URI | 1 | 3.322 | −1.752 | −0.087 | −0.02 | −0.01 | +0.424 | 2.765 |
| ATOM | 40 | O40 | URI | 1 | 1.098 | −2.469 | 0.441 | +0.19 | −0.02 | −0.227 | 2.765 |
| ATOM | 41 | C12 | URI | 1 | −1.986 | −6.972 | −6.160 | −0.36 | +0.04 | +0.113 | 2.765 |
| ATOM | 42 | O15 | URI | 1 | −2.074 | −6.797 | −7.578 | −0.14 | −0.13 | −0.537 | 2.765 |
| ATOM | 43 | H15 | URI | 1 | −1.536 | −6.006 | −7.771 | −0.24 | −0.27 | +0.424 | 2.765 |
| ATOM | 44 | C10 | URI | 1 | −2.529 | −8.385 | −5.846 | −0.40 | +0.08 | +0.113 | 2.765 |
| ATOM | 45 | O11 | URI | 1 | −3.561 | −8.715 | −6.747 | −0.23 | −0.56 | −0.537 | 2.765 |
| ATOM | 46 | H11 | URI | 1 | −3.094 | −8.971 | −7.569 | +0.07 | +0.30 | +0.424 | 2.765 |
| TER | | | | | | | | | | | |
| ENDMDL | | | | | | | | | | | |
| MODEL | 42 | | | | | | | | | | |
| USER | Run = 42 | | | | | | | | | | |
| USER | Cluster Rank = 25 | | | | | | | | | | |
| USER | Number of conformations in this cluster = 2 | | | | | | | | | | |
| USER | | | | | | | | | | | |
| USER | RMSD from reference structure = 4.064 A | | | | | | | | | | |
| USER | | | | | | | | | | | |
| USER | Estimated Free Energy of Binding = −5.25 kcal/mol [= (1) + (3)] | | | | | | | | | | |
| USER | Estimated Inhibition Constant, Ki = +1.43e−04 [Temperature = 298.15 K] | | | | | | | | | | |
| USER | | | | | | | | | | | |
| USER | Final Docked Energy = −8.08 kcal/mol [= (1) + (2)] | | | | | | | | | | |
| USER | | | | | | | | | | | |
| USER | (1) Final Intermolecular Energy = −9.92 kcal/mol | | | | | | | | | | |
| USER | (2) Final Internal Energy of Ligand = +1.83 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −4.421231 −2.595916 −1.735647 | | | | | | | | | | |
| USER | NEWDPF quat0 0.491085 −0.425063 −0.760367 −15.543889 | | | | | | | | | | |
| USER | NEWDPF ndihe 15 | | | | | | | | | | |
| USER | NEWDPF dihe0 −105.10 −166.13 −145.80 147.36 −112.17 120.92 −55.25 43.24 | | | | | | | | | | |
| | 3.19 177.00 −93.95 103.47 82.54 −89.22 44.60 | | | | | | | | | | |
| USER | | | | | | | | | | | |
| USER | | | | | x | y | z | vdW | Elec | q | RMS |
| ATOM | 1 | C2 | URI | 1 | −5.439 | −10.942 | −2.071 | −0.21 | +0.18 | +0.296 | 4.064 |
| ATOM | 2 | N3 | URI | 1 | −5.046 | −11.992 | −1.279 | −0.07 | −0.13 | −0.292 | 4.064 |
| ATOM | 3 | C4 | URI | 1 | −4.737 | −11.930 | 0.081 | −0.07 | +0.10 | +0.346 | 4.064 |
| ATOM | 4 | C5 | URI | 1 | −4.842 | −10.625 | 0.655 | −0.05 | +0.00 | +0.000 | 4.064 |
| ATOM | 5 | C6 | URI | 1 | −5.231 | −9.594 | −0.110 | −0.08 | −0.01 | −0.050 | 4.064 |
| ATOM | 6 | N1 | URI | 1 | −5.515 | −9.719 | −1.465 | −0.10 | +0.02 | +0.039 | 4.064 |
| ATOM | 7 | O8 | URI | 1 | −4.392 | −12.967 | 0.646 | +0.21 | −0.11 | −0.396 | 4.064 |
| ATOM | 8 | O7 | URI | 1 | −5.697 | −11.094 | −3.266 | −0.04 | −0.42 | −0.396 | 4.064 |
| ATOM | 9 | H3 | URI | 1 | −4.973 | −12.890 | −1.722 | +0.11 | +0.18 | +0.393 | 4.064 |
| ATOM | 10 | C9 | URI | 1 | −5.915 | −8.551 | −2.258 | −0.18 | +0.11 | −0.174 | 4.064 |
| ATOM | 11 | O14 | URI | 1 | −4.796 | −8.216 | −3.070 | −0.01 | −0.16 | −0.227 | 4.064 |
| ATOM | 12 | C13 | URI | 1 | −4.897 | −6.823 | −3.472 | −0.32 | +0.05 | +0.113 | 4.064 |
| ATOM | 13 | C16 | URI | 1 | −3.517 | −6.177 | −3.339 | −0.33 | +0.03 | +0.113 | 4.064 |
| ATOM | 14 | O17 | URI | 1 | −2.830 | −6.365 | −4.561 | −0.03 | −0.16 | −0.368 | 4.064 |
| ATOM | 15 | P18 | URI | 1 | −2.292 | −7.642 | −5.317 | −0.34 | +0.73 | +1.210 | 4.064 |
| ATOM | 16 | O19 | URI | 1 | −2.247 | −8.765 | −4.338 | +0.10 | −0.47 | −0.850 | 4.064 |
| ATOM | 17 | O20 | URI | 1 | −3.093 | −7.825 | −6.569 | −0.29 | −1.07 | −0.850 | 4.064 |
| ATOM | 18 | O21 | URI | 1 | −0.821 | −7.074 | −5.556 | +0.06 | −0.12 | −0.510 | 4.064 |
| ATOM | 19 | P22 | URI | 1 | 0.333 | −6.578 | −4.567 | −0.29 | +0.39 | +1.210 | 4.064 |
| ATOM | 20 | O25 | URI | 1 | −0.075 | −6.484 | −3.143 | +0.13 | −0.28 | −0.850 | 4.064 |
| ATOM | 21 | O24 | URI | 1 | 1.450 | −7.470 | −4.969 | −0.01 | −0.40 | −0.850 | 4.064 |
| ATOM | 22 | O23 | URI | 1 | 0.612 | −5.047 | −4.999 | +0.08 | −0.12 | −0.368 | 4.064 |
| ATOM | 23 | C37 | URI | 1 | 1.876 | −5.150 | −5.614 | −0.34 | +0.12 | +0.227 | 4.064 |
| ATOM | 24 | C45 | URI | 1 | 2.943 | −4.303 | −4.853 | −0.39 | +0.19 | +0.211 | 4.064 |
| ATOM | 25 | N41 | URI | 1 | 2.944 | −4.687 | −3.415 | −0.21 | −0.52 | −0.650 | 4.064 |
| ATOM | 26 | H41 | URI | 1 | 2.200 | −4.268 | −2.856 | +0.10 | +0.30 | +0.440 | 4.064 |
| ATOM | 27 | C49 | URI | 1 | 3.789 | −5.582 | −2.891 | −0.58 | +0.40 | +0.396 | 4.064 |
| ATOM | 28 | O38 | URI | 1 | 4.690 | −6.132 | −3.526 | −0.28 | −0.54 | −0.396 | 4.064 |
| ATOM | 29 | C50 | URI | 1 | 3.576 | −5.907 | −1.416 | −0.59 | +0.00 | +0.000 | 4.064 |
| ATOM | 30 | C44 | URI | 1 | 2.684 | −2.781 | −5.076 | −0.47 | +0.12 | +0.113 | 4.064 |
| ATOM | 31 | O47 | URI | 1 | 3.760 | −2.014 | −4.542 | −0.08 | −0.58 | −0.537 | 4.064 |
| ATOM | 32 | H47 | URI | 1 | 3.389 | −1.423 | −3.853 | +0.06 | +0.35 | +0.424 | 4.064 |
| ATOM | 33 | C43 | URI | 1 | 2.526 | −2.418 | −6.575 | −0.57 | +0.13 | +0.113 | 4.064 |
| ATOM | 34 | O48 | URI | 1 | 2.146 | −1.051 | −6.680 | −0.23 | −0.92 | −0.537 | 4.064 |
| ATOM | 35 | H48 | URI | 1 | 1.312 | −1.064 | −7.189 | +0.02 | +0.71 | +0.424 | 4.064 |
| ATOM | 36 | C46 | URI | 1 | 1.469 | −3.330 | −7.257 | −0.49 | +0.06 | +0.113 | 4.064 |
| ATOM | 37 | C42 | URI | 1 | 1.375 | −3.126 | −8.777 | −0.75 | +0.06 | +0.113 | 4.064 |
| ATOM | 38 | O39 | URI | 1 | 0.409 | −4.017 | −9.323 | −0.22 | −0.08 | −0.537 | 4.064 |
| ATOM | 39 | H39 | URI | 1 | −0.310 | −4.126 | −8.659 | −0.11 | −0.29 | +0.424 | 4.064 |
| ATOM | 40 | O40 | URI | 1 | 1.796 | −4.730 | −7.009 | +0.03 | −0.10 | −0.227 | 4.064 |
| ATOM | 41 | C12 | URI | 1 | −5.940 | −6.204 | −2.543 | −0.55 | +0.01 | +0.113 | 4.064 |
| ATOM | 42 | O15 | URI | 1 | −7.188 | −6.082 | −3.232 | −0.31 | −0.03 | −0.537 | 4.064 |
| ATOM | 43 | H15 | URI | 1 | −7.479 | −6.999 | −3.396 | +0.02 | +0.29 | +0.424 | 4.064 |
| ATOM | 44 | C10 | URI | 1 | −6.133 | −7.273 | −1.443 | −0.32 | +0.02 | +0.113 | 4.064 |
| ATOM | 45 | O11 | URI | 1 | −7.451 | −7.221 | −0.948 | +0.00 | +0.01 | −0.537 | 4.064 |
| ATOM | 46 | H11 | URI | 1 | −7.415 | −6.556 | −0.229 | +0.09 | −0.10 | +0.424 | 4.064 |
| TER | | | | | | | | | | | |
| ENDMDL | | | | | | | | | | | |
| MODEL | 40 | | | | | | | | | | |
| USER | Run = 40 | | | | | | | | | | |
| USER | Cluster Rank = 25 | | | | | | | | | | |
| USER | Number of conformations in this cluster = 2 | | | | | | | | | | |
| USER | | | | | | | | | | | |
| USER | RMSD from reference structure = 3.932 A | | | | | | | | | | |
| USER | | | | | | | | | | | |
| USER | Estimated Free Energy of Binding = −3.57 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −6.19 kcal/mol [= (1) + (2)] | | | | | | | | | | |
| USER | | | | | | | | | | | |
| USER | (1) Final Intermolecular Energy = −8.24 kcal/mol | | | | | | | | | | |
| USER | (2) Final Internal Energy of Ligand = +2.05 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −2.994607 −2.352659 −1.172688 | | | | | | | | | | |
| USER | NEWDPF quat0 −0.223679 −0.666115 −0.711519 −5.938739 | | | | | | | | | | |
| USER | NEWDPF ndihe 15 | | | | | | | | | | |
| USER | NEWDPF dihe0 166.81 34.55 45.69 76.52 174.28 −152.53 −52.12 32.99 −67.27 | | | | | | | | | | |
| | −147.08 −2.44 −15.78 67.69 −99.24 −1.96 | | | | | | | | | | |
| USER | | | | | | | | | | | |
| USER | | | | | x | y | z | vdW | Elec | q | RMS |
| ATOM | 1 | C2 | URI | 1 | −5.108 | −10.329 | −2.823 | −0.24 | +0.23 | +0.296 | 3.932 |
| ATOM | 2 | N3 | URI | 1 | −4.899 | −11.530 | −2.193 | −0.12 | −0.17 | −0.292 | 3.932 |
| ATOM | 3 | C4 | URI | 1 | −4.652 | −11.715 | −0.831 | −0.10 | +0.13 | +0.346 | 3.932 |
| ATOM | 4 | C5 | URI | 1 | −4.609 | −10.509 | −0.065 | −0.07 | +0.00 | +0.000 | 3.932 |
| ATOM | 5 | C6 | URI | 1 | −4.818 | −9.331 | −0.672 | −0.10 | −0.01 | −0.050 | 3.932 |
| ATOM | 6 | N1 | URI | 1 | −5.049 | −9.211 | −2.038 | −0.12 | +0.02 | +0.039 | 3.932 |
| ATOM | 7 | O8 | URI | 1 | −4.478 | −12.863 | −0.424 | +0.20 | −0.14 | −0.396 | 3.932 |
| ATOM | 8 | O7 | URI | 1 | −5.325 | −10.262 | −4.034 | +0.14 | −0.49 | −0.396 | 3.932 |
| ATOM | 9 | H3 | URI | 1 | −4.926 | −12.353 | −2.766 | +0.10 | +0.25 | +0.393 | 3.932 |
| ATOM | 10 | C9 | URI | 1 | −5.248 | −7.893 | −2.651 | −0.31 | +0.10 | +0.174 | 3.932 |
| ATOM | 11 | O14 | URI | 1 | −4.066 | −7.147 | −2.391 | +0.13 | −0.07 | −0.227 | 3.932 |
| ATOM | 12 | C13 | URI | 1 | −4.365 | −5.728 | −2.482 | −0.37 | +0.01 | +0.113 | 3.932 |
| ATOM | 13 | C16 | URI | 1 | −3.677 | −5.010 | −1.320 | −0.29 | −0.01 | +0.113 | 3.932 |
| ATOM | 14 | O17 | URI | 1 | −2.310 | −4.857 | −1.653 | +0.13 | −0.02 | −0.368 | 3.932 |
| ATOM | 15 | P18 | URI | 1 | −1.066 | −5.825 | −1.594 | −0.13 | +0.25 | +1.210 | 3.932 |
| ATOM | 16 | O19 | URI | 1 | 0.080 | −5.060 | −1.025 | +0.19 | −0.22 | −0.850 | 3.932 |
| ATOM | 17 | O20 | URI | 1 | −1.482 | −7.091 | −0.909 | +0.18 | −0.16 | −0.850 | 3.932 |
| ATOM | 18 | O21 | URI | 1 | −0.897 | −5.959 | −3.173 | +0.15 | −0.15 | −0.510 | 3.932 |
| ATOM | 19 | P22 | URI | 1 | 0.365 | −6.227 | −4.119 | −0.27 | +0.42 | +1.210 | 3.932 |
| ATOM | 20 | O25 | URI | 1 | 1.454 | −7.011 | −3.484 | +0.03 | −0.37 | −0.850 | 3.932 |
| ATOM | 21 | O24 | URI | 1 | −0.305 | −6.732 | −5.344 | +0.08 | −0.17 | −0.850 | 3.932 |
| ATOM | 22 | O23 | URI | 1 | 1.015 | −4.768 | −4.357 | +0.09 | −0.21 | −0.368 | 3.932 |
| ATOM | 23 | O37 | URI | 1 | 2.098 | −5.075 | −5.206 | −0.34 | +0.14 | +0.227 | 3.932 |
| ATOM | 24 | C45 | URI | 1 | 3.430 | −4.476 | −4.657 | −0.45 | +0.21 | +0.211 | 3.932 |
| ATOM | 25 | N41 | URI | 1 | 3.629 | −4.920 | −3.250 | −0.30 | −0.63 | −0.650 | 3.932 |
| ATOM | 26 | H41 | URI | 1 | 3.060 | −4.431 | −2.560 | +0.09 | +0.35 | +0.440 | 3.932 |
| ATOM | 27 | C49 | URI | 1 | 4.419 | −5.942 | −2.904 | −0.66 | +0.52 | +0.396 | 3.932 |
| ATOM | 28 | O38 | URI | 1 | 5.110 | −6.579 | −3.701 | −0.28 | −0.60 | −0.396 | 3.932 |
| ATOM | 29 | C50 | URI | 1 | 4.423 | −6.300 | −1.421 | −0.65 | +0.00 | +0.000 | 3.932 |
| ATOM | 30 | C44 | URI | 1 | 3.429 | −2.923 | −4.811 | −0.50 | +0.12 | +0.113 | 3.932 |
| ATOM | 31 | O47 | URI | 1 | 4.711 | −2.394 | −4.488 | −0.13 | −0.72 | −0.537 | 3.932 |
| ATOM | 32 | H47 | URI | 1 | 4.825 | −1.563 | −4.995 | +0.05 | +0.63 | +0.424 | 3.932 |
| ATOM | 33 | C43 | URI | 1 | 3.064 | −2.468 | −6.247 | −0.59 | +0.14 | +0.113 | 3.932 |
| ATOM | 34 | O48 | URI | 1 | 2.938 | −1.051 | −6.263 | −0.15 | −0.78 | −0.537 | 3.932 |
| ATOM | 35 | H48 | URI | 1 | 3.859 | −0.721 | −6.264 | +0.02 | +0.58 | +0.424 | 3.932 |
| ATOM | 36 | C46 | URI | 1 | 1.744 | −3.137 | −6.721 | −0.53 | +0.09 | +0.113 | 3.932 |
| ATOM | 37 | C42 | URI | 1 | 1.406 | −2.848 | −8.193 | −0.71 | +0.07 | +0.113 | 3.932 |
| ATOM | 38 | O39 | URI | 1 | 0.527 | −1.732 | −8.274 | −0.36 | −0.46 | −0.537 | 3.932 |
| ATOM | 39 | H39 | URI | 1 | −0.194 | −1.962 | −8.904 | +0.03 | +0.26 | +0.424 | 3.932 |
| ATOM | 40 | O40 | URI | 1 | 1.839 | −4.583 | −6.555 | +0.04 | −0.11 | −0.227 | 3.932 |
| ATOM | 41 | C12 | URI | 1 | −5.889 | −5.628 | −2.430 | −0.60 | +0.00 | +0.113 | 3.932 |
| ATOM | 42 | O15 | URI | 1 | −6.403 | −5.419 | −3.749 | −0.20 | +0.05 | −0.537 | 3.932 |
| ATOM | 43 | H15 | URI | 1 | −5.617 | −5.321 | −4.317 | +0.07 | −0.02 | +0.424 | 3.932 |
| ATOM | 44 | C10 | URI | 1 | −6.335 | −7.042 | −1.990 | −0.43 | +0.03 | +0.113 | 3.932 |
| ATOM | 45 | O11 | URI | 1 | −7.598 | −7.339 | −2.537 | −0.06 | −0.19 | −0.537 | 3.932 |
| ATOM | 46 | H11 | URI | 1 | −7.936 | −8.076 | −1.987 | +0.07 | +0.15 | +0.424 | 3.932 |
| TER | | | | | | | | | | | |
| ENDMDL | | | | | | | | | | | |
| MODEL | 4 | | | | | | | | | | |
| USER | Run = 4 | | | | | | | | | | |
| USER | Cluster Rank = 26 | | | | | | | | | | |
| USER | Number of conformations in this cluster = 1 | | | | | | | | | | |
| USER | | | | | | | | | | | |
| USER | RMSD from reference structure = 3.496 A | | | | | | | | | | |
| USER | | | | | | | | | | | |
| USER | Estimated Free Energy of Binding = −6.12 kcal/mol [= (1) + (3)] | | | | | | | | | | |
| USER | Estimated Inhibition Constant, Ki = +3.26e−05 [Temperature = 298.15 K] | | | | | | | | | | |
| USER | | | | | | | | | | | |
| USER | Final Docked Energy = −8.06 kcal/mol [= (1) + (2)] | | | | | | | | | | |
| USER | | | | | | | | | | | |
| USER | (1) Final Intermolecular Energy = −10.79 kcal/mol | | | | | | | | | | |
| USER | (2) Final Internal Energy of Ligand = +2.73 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 5.777373 −3.742958 −5.614121 | | | | | | | | | | |
| USER | NEWDPF quat0 −0.373944 −0.595390 −0.711110 44.815710 | | | | | | | | | | |
| USER | NEWDPF ndihe 15 | | | | | | | | | | |
| USER | NEWDPF dihe0 −178.30 −166.94 179.64 −105.88 −141.16 −145.33 −72.12 39.48 | | | | | | | | | | |
| | 2.46 −2.12 −12.15 −151.19 −82.48 78.87 −3.97 | | | | | | | | | | |
| USER | | | | | | | | | | | |
| USER | | | | | x | y | z | vdW | Elec | q | RMS |
| ATOM | 1 | C2 | URI | 1 | −0.040 | −9.615 | −7.191 | −0.71 | +0.20 | +0.296 | 3.496 |
| ATOM | 2 | N3 | URI | 1 | −0.889 | −10.361 | −6.413 | −0.39 | −0.21 | −0.292 | 3.496 |
| ATOM | 3 | C4 | URI | 1 | −1.352 | −10.025 | −5.140 | −0.44 | +0.22 | +0.346 | 3.496 |
| ATOM | 4 | C5 | URI | 1 | −0.856 | −8.783 | −4.636 | −0.31 | +0.00 | +0.000 | 3.496 |
| ATOM | 5 | C6 | URI | 1 | −0.030 | −8.044 | −5.391 | −0.39 | −0.02 | −0.050 | 3.496 |
| ATOM | 6 | N1 | URI | 1 | 0.404 | −8.440 | −6.652 | −0.36 | +0.02 | +0.039 | 3.496 |
| ATOM | 7 | O8 | URI | 1 | −2.107 | −10.813 | −4.570 | −0.05 | −0.30 | −0.396 | 3.496 |
| ATOM | 8 | O7 | URI | 1 | 0.312 | −9.991 | −8.310 | −0.24 | −0.36 | −0.396 | 3.496 |
| ATOM | 9 | H3 | URI | 1 | −1.207 | −11.233 | −6.798 | +0.06 | +0.32 | +0.393 | 3.496 |
| ATOM | 10 | C9 | URI | 1 | 1.327 | −7.600 | −7.425 | −0.48 | +0.08 | +0.174 | 3.496 |
| ATOM | 11 | O14 | URI | 1 | 2.483 | −7.425 | −6.614 | +0.03 | −0.13 | −0.227 | 3.496 |
| ATOM | 12 | C13 | URI | 1 | 3.190 | −6.230 | −7.041 | −0.44 | +0.09 | +0.113 | 3.496 |
| ATOM | 13 | C16 | URI | 1 | 3.631 | −5.455 | −5.798 | −0.52 | +0.11 | +0.113 | 3.496 |
| ATOM | 14 | O17 | URI | 1 | 2.752 | −4.357 | −5.638 | +0.07 | −0.31 | −0.368 | 3.496 |
| ATOM | 15 | P18 | URI | 1 | 1.913 | −3.811 | −4.418 | −0.31 | +1.06 | +1.210 | 3.496 |
| ATOM | 16 | O19 | URI | 1 | 2.776 | −2.863 | −3.659 | +0.05 | −0.75 | −0.850 | 3.496 |
| ATOM | 17 | O20 | URI | 1 | 0.597 | −3.315 | −4.933 | −0.26 | −1.40 | −0.850 | 3.496 |
| ATOM | 18 | O21 | URI | 1 | 1.832 | −5.205 | −3.648 | +0.12 | −0.30 | −0.510 | 3.496 |
| ATOM | 19 | P22 | URI | 1 | 1.910 | −5.606 | −2.102 | −0.30 | +0.60 | +1.210 | 3.496 |
| ATOM | 20 | O25 | URI | 1 | 1.628 | −7.036 | −1.822 | −0.01 | −0.29 | −0.850 | 3.496 |
| ATOM | 21 | O24 | URI | 1 | 3.218 | −5.013 | −1.724 | +0.00 | −0.66 | −0.850 | 3.496 |
| ATOM | 22 | O23 | URI | 1 | 0.681 | −4.819 | −1.411 | +0.18 | −0.13 | −0.368 | 3.496 |
| ATOM | 23 | C37 | URI | 1 | 1.358 | −3.839 | −0.658 | −0.17 | +0.07 | +0.227 | 3.496 |
| ATOM | 24 | C45 | URI | 1 | 1.002 | −3.950 | 0.857 | −0.11 | +0.03 | +0.211 | 3.496 |
| ATOM | 25 | N41 | URI | 1 | 1.253 | −5.341 | 1.324 | −0.07 | −0.13 | −0.650 | 3.496 |
| ATOM | 26 | H41 | URI | 1 | 0.430 | −5.944 | 1.341 | +0.11 | +0.06 | +0.440 | 3.496 |
| ATOM | 27 | C49 | URI | 1 | 2.469 | −5.813 | 1.614 | −0.26 | +0.12 | +0.396 | 3.496 |
| ATOM | 28 | O38 | URI | 1 | 3.496 | −5.133 | 1.589 | +0.07 | −0.18 | −0.396 | 3.496 |
| ATOM | 29 | C50 | URI | 1 | 2.531 | −7.291 | 1.990 | −0.33 | +0.00 | +0.000 | 3.496 |
| ATOM | 30 | C44 | URI | 1 | −0.465 | −3.480 | 1.104 | −0.11 | −0.01 | +0.113 | 3.496 |
| ATOM | 31 | O47 | URI | 1 | −0.730 | −3.401 | 2.502 | +0.18 | +0.09 | −0.537 | 3.496 |
| ATOM | 32 | H47 | URI | 1 | −1.571 | −2.910 | 2.617 | +0.10 | −0.15 | +0.424 | 3.496 |
| ATOM | 33 | C43 | URI | 1 | −0.760 | −2.094 | 0.475 | −0.22 | −0.02 | +0.113 | 3.496 |
| ATOM | 34 | O48 | URI | 1 | −2.147 | −1.812 | 0.614 | −0.01 | +0.29 | −0.537 | 3.496 |
| ATOM | 35 | H48 | URI | 1 | −2.457 | −1.637 | −0.297 | −0.21 | −0.27 | +0.424 | 3.496 |
| ATOM | 36 | C46 | URI | 1 | −0.347 | −2.067 | −1.022 | −0.25 | +0.02 | +0.113 | 3.496 |
| ATOM | 37 | C42 | URI | 1 | −0.470 | −0.676 | −1.665 | −0.37 | +0.04 | +0.113 | 3.496 |
| ATOM | 38 | O39 | URI | 1 | −1.788 | −0.494 | −2.168 | −0.02 | −0.05 | −0.537 | 3.496 |
| ATOM | 39 | H39 | URI | 1 | −2.298 | −1.316 | −1.982 | −0.21 | −0.16 | +0.424 | 3.496 |
| ATOM | 40 | O40 | URI | 1 | 1.038 | −2.506 | −1.157 | +0.15 | −0.08 | −0.227 | 3.496 |
| ATOM | 41 | C12 | URI | 1 | 2.197 | −5.456 | −7.908 | −0.59 | +0.06 | +0.113 | 3.496 |
| ATOM | 42 | O15 | URI | 1 | 2.508 | −5.655 | −9.291 | −0.16 | −0.40 | −0.537 | 3.496 |
| ATOM | 43 | H15 | URI | 1 | 1.776 | −5.234 | −9.780 | +0.04 | +0.25 | +0.424 | 3.496 |
| ATOM | 44 | C10 | URI | 1 | 0.848 | −6.163 | −7.642 | −0.43 | +0.02 | +0.113 | 3.496 |
| ATOM | 45 | O11 | URI | 1 | 0.029 | −6.081 | −8.785 | −0.11 | −0.05 | −0.537 | 3.496 |
| ATOM | 46 | H11 | URI | 1 | −0.744 | −5.553 | −8.494 | −0.27 | −0.25 | +0.424 | 3.496 |
| TER | | | | | | | | | | | |
| ENDMDL | | | | | | | | | | | |
| MODEL | 29 | | | | | | | | | | |
| USER | Run = 29 | | | | | | | | | | |
| USER | Cluster Rank = 27 | | | | | | | | | | |
| USER | Number of conformations in this cluster = 1 | | | | | | | | | | |
| USER | | | | | | | | | | | |
| USER | RMSD from reference structure = 3.818 A | | | | | | | | | | |
| USER | | | | | | | | | | | |
| USER | Estimated Free Energy of Binding = −3.91 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −8.05 kcal/mol [= (1) + (2)] | | | | | | | | | | |
| USER | | | | | | | | | | | |
| USER | (1) Final Intermolecular Energy = −8.58 kcal/mol | | | | | | | | | | |
| USER | (2) Final Internal Energy of Ligand = +0.53 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 2.734475 −4.604975 −0.879625 | | | | | | | | | | |
| USER | NEWDPF quat0 0.287351 0.567231 −0.771802 23.111460 | | | | | | | | | | |
| USER | NEWDPF ndihe 15 | | | | | | | | | | |
| USER | NEWDPF dihe0 172.70 36.53 −80.43 −87.68 180.00 180.00 −8.03 35.40 −32.96 | | | | | | | | | | |
| | −114.27 −14.17 160.07 −98.17 −68.13 −4.02 | | | | | | | | | | |
| USER | | | | | | | | | | | |
| USER | | | | | x | y | z | vdW | Elec | q | RMS |
| ATOM | 1 | C2 | URI | 1 | −2.471 | −11.029 | −2.443 | −0.30 | +0.15 | +0.296 | 3.818 |
| ATOM | 2 | N3 | URI | 1 | −2.642 | −12.281 | −1.908 | −0.19 | −0.15 | −0.292 | 3.818 |
| ATOM | 3 | C4 | URI | 1 | −2.284 | −12.684 | −0.620 | −0.32 | +0.16 | +0.346 | 3.818 |
| ATOM | 4 | C5 | URI | 1 | −1.672 | −11.664 | 0.173 | −0.29 | +0.00 | +0.000 | 3.818 |
| ATOM | 5 | C6 | URI | 1 | −1.505 | −10.436 | −0.340 | −0.18 | −0.02 | −0.050 | 3.818 |
| ATOM | 6 | N1 | URI | 1 | −1.875 | −10.099 | −1.638 | −0.19 | +0.02 | +0.039 | 3.818 |
| ATOM | 7 | O8 | URI | 1 | −2.500 | −13.851 | −0.296 | +0.10 | −0.20 | −0.396 | 3.818 |
| ATOM | 8 | O7 | URI | 1 | −2.824 | −10.763 | −3.593 | +0.06 | −0.26 | −0.396 | 3.818 |
| ATOM | 9 | H3 | URI | 1 | −3.065 | −12.973 | −2.500 | +0.10 | +0.23 | +0.393 | 3.818 |
| ATOM | 10 | C9 | URI | 1 | −1.648 | −8.744 | −2.153 | −0.25 | +0.06 | +0.174 | 3.818 |
| ATOM | 11 | O14 | URI | 1 | −0.252 | −8.499 | −2.032 | +0.03 | −0.07 | −0.227 | 3.818 |
| ATOM | 12 | C13 | URI | 1 | −0.019 | −7.064 | −2.013 | −0.26 | +0.03 | +0.113 | 3.818 |
| ATOM | 13 | C16 | URI | 1 | 1.016 | −6.754 | −0.931 | −0.31 | +0.03 | +0.113 | 3.818 |
| ATOM | 14 | O17 | URI | 1 | 2.213 | −6.361 | −1.575 | +0.00 | −0.17 | −0.368 | 3.818 |
| ATOM | 15 | P18 | URI | 1 | 3.013 | −5.002 | −1.639 | −0.37 | +0.85 | +1.210 | 3.818 |
| ATOM | 16 | O19 | URI | 1 | 2.973 | −4.386 | −0.283 | +0.08 | −0.45 | −0.850 | 3.818 |
| ATOM | 17 | O20 | URI | 1 | 4.351 | −5.269 | −2.257 | −0.30 | −1.21 | −0.850 | 3.818 |
| ATOM | 18 | O21 | URI | 1 | 1.982 | −4.259 | −2.602 | +0.12 | −0.32 | −0.510 | 3.818 |
| ATOM | 19 | P22 | URI | 1 | 1.400 | −2.771 | −2.640 | −0.26 | +0.98 | +1.210 | 3.818 |
| ATOM | 20 | O25 | URI | 1 | 2.425 | −1.714 | −2.830 | +0.03 | −0.54 | −0.850 | 3.818 |
| ATOM | 21 | O24 | URI | 1 | 0.295 | −2.926 | −3.620 | −0.07 | −1.97 | −0.850 | 3.818 |
| ATOM | 22 | O23 | URI | 1 | 0.838 | −2.512 | −1.148 | +0.16 | −0.13 | −0.368 | 3.818 |
| ATOM | 23 | C37 | URI | 1 | −0.293 | −1.710 | −1.406 | −0.30 | +0.07 | +0.227 | 3.818 |
| ATOM | 24 | C45 | URI | 1 | −0.239 | −0.383 | −0.588 | −0.32 | −0.01 | +0.211 | 3.818 |
| ATOM | 25 | N41 | URI | 1 | 1.055 | 0.306 | −0.849 | −0.19 | −0.01 | −0.650 | 3.818 |
| ATOM | 26 | H41 | URI | 1 | 1.846 | −0.314 | −1.022 | +0.10 | +0.04 | +0.440 | 3.818 |
| ATOM | 27 | C49 | URI | 1 | 1.175 | 1.634 | −0.948 | −0.48 | −0.05 | +0.396 | 3.818 |
| ATOM | 28 | O38 | URI | 1 | 0.248 | 2.426 | −0.770 | −0.05 | +0.12 | −0.396 | 3.818 |
| ATOM | 29 | C50 | URI | 1 | 2.567 | 2.146 | −1.308 | −0.49 | +0.00 | +0.000 | 3.818 |
| ATOM | 30 | C44 | URI | 1 | −0.480 | −0.670 | 0.927 | −0.27 | −0.04 | +0.113 | 3.818 |
| ATOM | 31 | O47 | URI | 1 | −0.618 | 0.552 | 1.646 | +0.05 | +0.39 | −0.537 | 3.818 |
| ATOM | 32 | H47 | URI | 1 | −1.105 | 0.355 | 2.474 | −0.09 | −0.57 | +0.424 | 3.818 |
| ATOM | 33 | C43 | URI | 1 | −1.748 | −1.526 | 1.175 | −0.41 | −0.06 | +0.113 | 3.818 |
| ATOM | 34 | O48 | URI | 1 | −1.810 | −1.866 | 2.555 | +0.00 | +0.36 | −0.537 | 3.818 |
| ATOM | 35 | H48 | URI | 1 | −2.158 | −1.064 | 2.993 | −0.23 | −0.55 | +0.424 | 3.818 |
| ATOM | 36 | C46 | URI | 1 | −1.731 | −2.809 | 0.299 | −0.27 | −0.03 | +0.113 | 3.818 |
| ATOM | 37 | C42 | URI | 1 | −3.031 | −3.624 | 0.383 | −0.25 | −0.05 | +0.113 | 3.818 |
| ATOM | 38 | O39 | URI | 1 | −2.742 | −4.946 | 0.824 | +0.16 | +0.07 | −0.537 | 3.818 |
| ATOM | 39 | H39 | URI | 1 | −2.944 | −4.994 | 1.786 | +0.11 | −0.07 | +0.424 | 3.818 |
| ATOM | 40 | O40 | URI | 1 | −1.515 | −2.446 | −1.098 | +0.07 | +0.01 | −0.227 | 3.818 |
| ATOM | 41 | C12 | URI | 1 | −1.382 | −6.432 | −1.735 | −0.14 | +0.02 | +0.113 | 3.818 |
| ATOM | 42 | O15 | URI | 1 | −1.936 | −5.927 | −2.954 | +0.14 | −0.13 | −0.537 | 3.818 |
| ATOM | 43 | H15 | URI | 1 | −2.703 | −5.388 | −2.683 | +0.11 | +0.06 | +0.424 | 3.818 |
| ATOM | 44 | C10 | URI | 1 | −2.260 | −7.629 | −1.302 | −0.13 | +0.02 | +0.113 | 3.818 |
| ATOM | 45 | O11 | URI | 1 | −3.600 | −7.401 | −1.674 | +0.18 | −0.12 | −0.537 | 3.818 |
| ATOM | 46 | H11 | URI | 1 | −4.122 | −7.949 | −1.052 | +0.11 | +0.09 | +0.424 | 3.818 |
| TER | | | | | | | | | | | |
| ENDMDL | | | | | | | | | | | |
| MODEL | 13 | | | | | | | | | | |
| USER | Run = 13 | | | | | | | | | | |
| USER | Cluster Rank = 28 | | | | | | | | | | |
| USER | Number of conformations in this cluster = 1 | | | | | | | | | | |
| USER | | | | | | | | | | | |
| USER | RMSD from reference structure = 3.887 A | | | | | | | | | | |
| USER | | | | | | | | | | | |
| USER | Estimated Free Energy of Binding = −5.65 kcal/mol [= (1) + (3)] | | | | | | | | | | |
| USER | Estimated Inhibition Constant, Ki = +7.17e−05 [Temperature = 298.15 K ] | | | | | | | | | | |
| USER | | | | | | | | | | | |
| USER | Final Docked Energy = −7.94 kcal/mol [= (1) + (2)] | | | | | | | | | | |
| USER | | | | | | | | | | | |
| USER | (1) Final Intermolecular Energy = −10.32 kcal/mol | | | | | | | | | | |
| USER | (2) Final Internal Energy of Ligand = +2.38 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −0.279451 −2.553801 −5.404457 | | | | | | | | | | |
| USER | NEWDPF quat0 0.788751 0.240877 −0.565553 −115.411093 | | | | | | | | | | |
| USER | NEWDPF ndihe 15 | | | | | | | | | | |
| USER | NEWDPF dihe0 −81.13 −130.23 −94.40 −104.04 −73.67 −177.63 11.61 −28.82 | | | | | | | | | | |
| | −2.60 −162.39 −26.87 39.07 −5.92 −15.10 29.49 | | | | | | | | | | |
| USER | | | | | | | | | | | |
| USER | | | | | x | y | z | vdW | Elec | q | RMS |
| ATOM | 1 | C2 | URI | 1 | 1.801 | −2.704 | 2.748 | −0.08 | +0.00 | +0.296 | 3.887 |
| ATOM | 2 | N3 | URI | 1 | 1.551 | −1.885 | 3.820 | −0.04 | +0.04 | −0.292 | 3.887 |
| ATOM | 3 | C4 | URI | 1 | 0.476 | −1.004 | 3.952 | −0.19 | −0.16 | +0.346 | 3.887 |
| ATOM | 4 | C5 | URI | 1 | −0.413 | −0.979 | 2.834 | −0.22 | +0.00 | +0.000 | 3.887 |
| ATOM | 5 | C6 | URI | 1 | −0.179 | −1.780 | 1.783 | −0.18 | +0.01 | −0.050 | 3.887 |
| ATOM | 6 | N1 | URI | 1 | 0.919 | −2.630 | 1.707 | −0.04 | +0.00 | +0.039 | 3.887 |
| ATOM | 7 | O8 | URI | 1 | 0.401 | −0.331 | 4.980 | +0.09 | +0.24 | −0.396 | 3.887 |
| ATOM | 8 | O7 | URI | 1 | 2.777 | −3.456 | 2.721 | +0.19 | −0.07 | −0.396 | 3.887 |
| ATOM | 9 | H3 | URI | 1 | 2.205 | −1.923 | 4.581 | +0.12 | −0.02 | +0.393 | 3.887 |
| ATOM | 10 | C9 | URI | 1 | 1.136 | −3.469 | 0.522 | −0.12 | +0.03 | +0.174 | 3.887 |
| ATOM | 11 | O14 | URI | 1 | 1.950 | −2.709 | −0.362 | +0.14 | −0.06 | −0.227 | 3.887 |
| ATOM | 12 | C13 | URI | 1 | 1.782 | −3.208 | −1.717 | −0.23 | +0.06 | +0.113 | 3.887 |
| ATOM | 13 | C16 | URI | 1 | 1.667 | −2.014 | −2.665 | −0.34 | +0.09 | +0.113 | 3.887 |
| ATOM | 14 | O17 | URI | 1 | 1.902 | −2.480 | −3.980 | −0.06 | −0.43 | −0.368 | 3.887 |
| ATOM | 15 | P18 | URI | 1 | 2.437 | −1.790 | −5.294 | −0.63 | +1.53 | +1.210 | 3.887 |
| ATOM | 16 | O19 | URI | 1 | 1.297 | −1.064 | −5.923 | −0.59 | −1.82 | −0.850 | 3.887 |
| ATOM | 17 | O20 | URI | 1 | 3.671 | −1.013 | −4.954 | −0.18 | −0.95 | −0.850 | 3.887 |
| ATOM | 18 | O21 | URI | 1 | 2.714 | −3.149 | −6.082 | +0.01 | −0.52 | −0.510 | 3.887 |
| ATOM | 19 | P22 | URI | 1 | 2.258 | −3.695 | −7.514 | −0.56 | +0.90 | +1.210 | 3.887 |
| ATOM | 20 | O25 | URI | 1 | 1.665 | −2.665 | −8.404 | −0.17 | −0.67 | −0.850 | 3.887 |
| ATOM | 21 | O24 | URI | 1 | 3.475 | −4.435 | −7.933 | −0.11 | −0.91 | −0.850 | 3.887 |
| ATOM | 22 | O23 | URI | 1 | 1.032 | −4.697 | −7.199 | +0.24 | +0.00 | −0.368 | 3.887 |
| ATOM | 23 | C37 | URI | 1 | 1.571 | −5.938 | −7.597 | −0.53 | +0.08 | +0.227 | 3.887 |
| ATOM | 24 | C45 | URI | 1 | 0.653 | −7.118 | −7.151 | −0.53 | +0.06 | +0.211 | 3.887 |
| ATOM | 25 | N41 | URI | 1 | −0.732 | −6.882 | −7.644 | −0.26 | −0.04 | −0.650 | 3.887 |
| ATOM | 26 | H41 | URI | 1 | −0.911 | −5.938 | −7.988 | −0.26 | −0.28 | +0.440 | 3.887 |
| ATOM | 27 | C49 | URI | 1 | −1.659 | −7.842 | −7.727 | −0.39 | +0.17 | +0.396 | 3.887 |
| ATOM | 28 | O38 | URI | 1 | −1.487 | −9.001 | −7.346 | −0.01 | −0.22 | −0.396 | 3.887 |
| ATOM | 29 | C50 | URI | 1 | −2.991 | −7.422 | −8.342 | −0.68 | +0.00 | +0.000 | 3.887 |
| ATOM | 30 | C44 | URI | 1 | 0.723 | −7.303 | −5.603 | −0.42 | +0.04 | +0.113 | 3.887 |
| ATOM | 31 | O47 | URI | 1 | 0.039 | −8.492 | −5.216 | +0.03 | −0.26 | −0.537 | 3.887 |
| ATOM | 32 | H47 | URI | 1 | −0.870 | −8.237 | −4.950 | +0.10 | +0.18 | +0.424 | 3.887 |
| ATOM | 33 | C43 | URI | 1 | 2.180 | −7.390 | −5.080 | −0.49 | +0.06 | +0.113 | 3.887 |
| ATOM | 34 | O48 | URI | 1 | 2.158 | −7.406 | −3.658 | +0.04 | −0.27 | −0.537 | 3.887 |
| ATOM | 35 | H48 | URI | 1 | 2.167 | −6.463 | −3.400 | +0.09 | +0.23 | +0.424 | 3.887 |
| ATOM | 36 | C46 | URI | 1 | 3.026 | −6.194 | −5.598 | −0.50 | +0.08 | +0.113 | 3.887 |
| ATOM | 37 | C42 | URI | 1 | 4.518 | −6.302 | −5.243 | −0.62 | +0.13 | +0.113 | 3.887 |
| ATOM | 38 | O39 | URI | 1 | 4.903 | −5.187 | −4.446 | −0.16 | −0.76 | −0.537 | 3.887 |
| ATOM | 39 | H39 | URI | 1 | 4.700 | −5.403 | −3.507 | +0.04 | +0.60 | +0.424 | 3.887 |
| ATOM | 40 | O40 | URI | 1 | 2.913 | −6.105 | −7.050 | −0.01 | −0.16 | −0.227 | 3.887 |
| ATOM | 41 | C12 | URI | 1 | 0.519 | −4.068 | −1.680 | −0.16 | +0.05 | +0.113 | 3.887 |
| ATOM | 42 | O15 | URI | 1 | 0.880 | −5.453 | −1.637 | +0.15 | −0.19 | −0.537 | 3.887 |
| ATOM | 43 | H15 | URI | 1 | 0.037 | −5.932 | −1.530 | +0.11 | +0.12 | +0.424 | 3.887 |
| ATOM | 44 | C10 | URI | 1 | −0.120 | −3.725 | −0.315 | −0.13 | +0.02 | +0.113 | 3.887 |
| ATOM | 45 | O11 | URI | 1 | −0.826 | −4.840 | 0.179 | +0.20 | −0.04 | −0.537 | 3.887 |
| ATOM | 46 | H11 | URI | 1 | −1.381 | −5.130 | −0.574 | +0.11 | +0.04 | +0.424 | 3.887 |
| TER | | | | | | | | | | | |
| ENDMDL | | | | | | | | | | | |
| MODEL | 16 | | | | | | | | | | |
| USER | Run = 16 | | | | | | | | | | |
| USER | Cluster Rank = 29 | | | | | | | | | | |
| USER | Number of conformations in this cluster = 1 | | | | | | | | | | |
| USER | | | | | | | | | | | |
| USER | RMSD from reference structure = 2.612 A | | | | | | | | | | |
| USER | | | | | | | | | | | |
| USER | Estimated Free Energy of Binding = −5.13 kcal/mol [= (1) + (3)] | | | | | | | | | | |
| USER | Estimated Inhibition Constant, Ki = +1.74e−04 [Temperature = 298.15 K] | | | | | | | | | | |
| USER | | | | | | | | | | | |
| USER | Final Docked Energy = −7.90 kcal/mol [= (1) + (2)] | | | | | | | | | | |
| USER | | | | | | | | | | | |
| USER | (1) Final Intermolecular Energy = −9.80 kcal/mol | | | | | | | | | | |
| USER | (2) Final Internal Energy of Ligand = +1.89 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.462267 −4.497147 −3.154757 | | | | | | | | | | |
| USER | NEWDPF quat0 0.633712 −0.479467 −0.607059 7.115815 | | | | | | | | | | |
| USER | NEWDPF ndihe 15 | | | | | | | | | | |
| USER | NEWDPF dihe0 52.94 102.11 −92.55 −23.69 19.25 −157.53 1.84 16.21 25.14 | | | | | | | | | | |
| | −121.40 136.29 −178.65 85.65 −16.37 −39.64 | | | | | | | | | | |
| USER | | | | | | | | | | | |
| USER | | | | | x | y | z | vdW | Elec | q | RMS |
| ATOM | 1 | C2 | URI | 1 | −1.580 | −11.967 | −5.555 | −0.61 | +0.24 | +0.296 | 2.612 |
| ATOM | 2 | N3 | URI | 1 | −1.633 | −13.213 | −4.982 | −0.40 | −0.20 | −0.292 | 2.612 |
| ATOM | 3 | C4 | URI | 1 | −1.593 | −13.496 | −3.616 | −0.64 | +0.24 | +0.346 | 2.612 |
| ATOM | 4 | C5 | URI | 1 | −1.470 | −12.347 | −2.774 | −0.54 | +0.00 | +0.000 | 2.612 |
| ATOM | 5 | C6 | URI | 1 | −1.422 | −11.125 | −3.325 | −0.32 | −0.03 | −0.050 | 2.612 |
| ATOM | 6 | N1 | URI | 1 | −1.456 | −10.909 | −4.698 | −0.31 | +0.03 | +0.039 | 2.612 |
| ATOM | 7 | O8 | URI | 1 | −1.644 | −14.674 | −3.265 | −0.12 | −0.32 | −0.396 | 2.612 |
| ATOM | 8 | O7 | URI | 1 | −1.627 | −11.813 | −6.776 | −0.24 | −0.29 | −0.396 | 2.612 |
| ATOM | 9 | H3 | URI | 1 | −1.707 | −13.996 | −5.607 | −0.27 | +0.12 | +0.393 | 2.612 |
| ATOM | 10 | C9 | URI | 1 | −1.379 | −9.549 | −5.245 | −0.42 | +0.10 | +0.174 | 2.612 |
| ATOM | 11 | O14 | URI | 1 | −0.772 | −8.747 | −4.239 | +0.08 | −0.11 | −0.227 | 2.612 |
| ATOM | 12 | C13 | URI | 1 | −1.124 | −7.355 | −4.460 | −0.29 | +0.04 | +0.113 | 2.612 |
| ATOM | 13 | C16 | URI | 1 | −1.474 | −6.722 | −3.112 | −0.20 | +0.03 | +0.113 | 2.612 |
| ATOM | 14 | O17 | URI | 1 | −1.395 | −5.317 | −3.260 | +0.11 | −0.10 | −0.368 | 2.612 |
| ATOM | 15 | P18 | URI | 1 | −0.218 | −4.329 | −3.616 | −0.22 | +0.87 | +1.210 | 2.612 |
| ATOM | 16 | O19 | URI | 1 | 0.284 | −4.687 | −4.972 | +0.06 | −0.29 | −0.850 | 2.612 |
| ATOM | 17 | O20 | URI | 1 | −0.688 | −2.926 | −3.382 | −0.13 | −1.92 | −0.850 | 2.612 |
| ATOM | 18 | O21 | URI | 1 | 0.787 | −4.877 | −2.505 | +0.16 | −0.24 | −0.510 | 2.612 |
| ATOM | 19 | P22 | URI | 1 | 0.808 | −4.785 | −0.909 | −0.13 | +0.37 | +1.210 | 2.612 |
| ATOM | 20 | O25 | URI | 1 | −0.434 | −5.260 | −0.249 | +0.20 | −0.13 | −0.850 | 2.612 |
| ATOM | 21 | O24 | URI | 1 | 2.110 | −5.431 | −0.608 | +0.10 | −0.36 | −0.850 | 2.612 |
| ATOM | 22 | O23 | URI | 1 | 0.843 | −3.202 | −0.590 | +0.18 | −0.09 | −0.368 | 2.612 |
| ATOM | 23 | C37 | URI | 1 | 2.227 | −2.935 | −0.628 | −0.19 | +0.07 | +0.227 | 2.612 |
| ATOM | 24 | C45 | URI | 1 | 2.618 | −1.847 | 0.419 | −0.19 | +0.02 | +0.211 | 2.612 |
| ATOM | 25 | N41 | URI | 1 | 2.124 | −2.253 | 1.763 | −0.06 | −0.03 | −0.650 | 2.612 |
| ATOM | 26 | H41 | URI | 1 | 1.345 | −2.912 | 1.755 | +0.11 | +0.02 | +0.440 | 2.612 |
| ATOM | 27 | C49 | URI | 1 | 2.700 | −1.870 | 2.907 | −0.10 | +0.01 | +0.396 | 2.612 |
| ATOM | 28 | O38 | URI | 1 | 3.655 | −1.095 | 2.972 | +0.18 | −0.02 | −0.396 | 2.612 |
| ATOM | 29 | C50 | URI | 1 | 2.101 | −2.463 | 4.179 | −0.06 | +0.00 | +0.000 | 2.612 |
| ATOM | 30 | C44 | URI | 1 | 2.086 | −0.450 | −0.028 | −0.24 | +0.00 | +0.113 | 2.612 |
| ATOM | 31 | O47 | URI | 1 | 2.600 | 0.571 | 0.823 | +0.13 | +0.07 | −0.537 | 2.612 |
| ATOM | 32 | H47 | URI | 1 | 3.528 | 0.338 | 1.037 | +0.10 | −0.03 | +0.424 | 2.612 |
| ATOM | 33 | C43 | URI | 1 | 2.475 | −0.105 | −1.489 | −0.39 | +0.01 | +0.113 | 2.612 |
| ATOM | 34 | O48 | URI | 1 | 1.828 | 1.105 | −1.863 | −0.05 | +0.00 | −0.537 | 2.612 |
| ATOM | 35 | H48 | URI | 1 | 2.469 | 1.558 | −2.447 | −0.11 | −0.08 | +0.424 | 2.612 |
| ATOM | 36 | C46 | URI | 1 | 2.076 | −1.256 | −2.454 | −0.40 | +0.06 | +0.113 | 2.612 |
| ATOM | 37 | C42 | URI | 1 | 2.571 | −1.045 | −3.894 | −0.61 | +0.10 | +0.113 | 2.612 |
| ATOM | 38 | O39 | URI | 1 | 1.511 | −0.541 | −4.698 | −0.48 | −0.92 | −0.537 | 2.612 |
| ATOM | 39 | H39 | URI | 1 | 1.514 | 0.440 | −4.620 | −0.08 | +0.60 | +0.424 | 2.612 |
| ATOM | 40 | O40 | URI | 1 | 2.625 | −2.517 | −1.967 | +0.19 | −0.11 | −0.227 | 2.612 |
| ATOM | 41 | C12 | URI | 1 | −2.303 | −7.381 | −5.432 | −0.36 | +0.07 | +0.113 | 2.612 |
| ATOM | 42 | O15 | URI | 1 | −1.847 | −7.064 | −6.751 | −0.15 | −0.17 | −0.537 | 2.612 |
| ATOM | 43 | H15 | URI | 1 | −2.144 | −6.149 | −6.910 | −0.13 | −0.09 | +0.424 | 2.612 |
| ATOM | 44 | C10 | URI | 1 | −2.733 | −8.866 | −5.447 | −0.39 | +0.08 | +0.113 | 2.612 |
| ATOM | 45 | O11 | URI | 1 | −3.272 | −9.197 | −6.706 | −0.12 | −0.45 | −0.537 | 2.612 |
| ATOM | 46 | H11 | URI | 1 | −2.660 | −8.784 | −7.350 | +0.08 | +0.29 | +0.424 | 2.612 |
| TER | | | | | | | | | | | |
| ENDMDL | | | | | | | | | | | |
| MODEL | 22 | | | | | | | | | | |
| USER | Run = 22 | | | | | | | | | | |
| USER | Cluster Rank = 30 | | | | | | | | | | |
| USER | Number of conformations in this cluster = 1 | | | | | | | | | | |
| USER | | | | | | | | | | | |
| USER | RMSD from reference structure = 4.982 A | | | | | | | | | | |
| USER | | | | | | | | | | | |
| USER | Estimated Free Energy of Binding = −5.13 kcal/mol [= (1) + (3)] | | | | | | | | | | |
| USER | Estimated Inhibition Constant, Ki = +1.73e−04 [Temperature = 298.15 K] | | | | | | | | | | |
| USER | | | | | | | | | | | |
| USER | Final Docked Energy = −7.81 kcal/mol [= (1) + (2)] | | | | | | | | | | |
| USER | | | | | | | | | | | |
| USER | (1) Final Intermolecular Energy = −9.80 kcal/mol | | | | | | | | | | |
| USER | (2) Final Internal Energy of Ligand = +1.99 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −0.485394 −5.438017 −4.745988 | | | | | | | | | | |
| USER | NEWDPF quat0 0.413962 0.905599 −0.092341 −69.691357 | | | | | | | | | | |
| USER | NEWDPF ndihe 15 | | | | | | | | | | |
| USER | NEWDPF dihe0 −179.68 107.35 129.95 −180.00 −150.73 −179.67 −35.05 | | | | | | | | | | |
| | −7.92 13.45 −179.85 −18.23 25.20 −174.20 −127.80 −25.01 | | | | | | | | | | |
| USER | | | | | | | | | | | |
| USER | | | | | x | y | z | vdW | Elec | q | RMS |
| ATOM | 1 | C2 | URI | 1 | −1.487 | −13.758 | −3.978 | −0.69 | +0.22 | +0.296 | 4.982 |
| ATOM | 2 | N3 | URI | 1 | −2.226 | −14.565 | −3.150 | −0.33 | −0.22 | −0.292 | 4.982 |
| ATOM | 3 | C4 | URI | 1 | −3.394 | −14.207 | −2.475 | −0.30 | +0.20 | +0.346 | 4.982 |
| ATOM | 4 | C5 | URI | 1 | −3.813 | −12.858 | −2.687 | −0.26 | +0.00 | +0.000 | 4.982 |
| ATOM | 5 | C6 | URI | 1 | −3.100 | −12.062 | −3.499 | −0.26 | −0.04 | −0.050 | 4.982 |
| ATOM | 6 | N1 | URI | 1 | −1.936 | −12.476 | −4.136 | −0.36 | +0.03 | +0.039 | 4.982 |
| ATOM | 7 | O8 | URI | 1 | −3.934 | −15.052 | −1.762 | +0.14 | −0.18 | −0.396 | 4.982 |
| ATOM | 8 | O7 | URI | 1 | −0.468 | −14.164 | −4.538 | −0.38 | −0.33 | −0.396 | 4.982 |
| ATOM | 9 | H3 | URI | 1 | −1.893 | −15.503 | −3.018 | +0.07 | +0.33 | +0.393 | 4.982 |
| ATOM | 10 | C9 | URI | 1 | −1.184 | −11.555 | −4.996 | −0.57 | +0.13 | +0.174 | 4.982 |
| ATOM | 11 | O14 | URI | 1 | −1.276 | −10.273 | −4.386 | +0.03 | −0.14 | −0.227 | 4.982 |
| ATOM | 12 | C13 | URI | 1 | −1.036 | −9.248 | −5.387 | −0.42 | +0.06 | +0.113 | 4.982 |
| ATOM | 13 | C16 | URI | 1 | −2.056 | −8.126 | −5.189 | −0.35 | +0.06 | +0.113 | 4.982 |
| ATOM | 14 | O17 | URI | 1 | −2.037 | −7.759 | −3.822 | +0.13 | −0.15 | −0.368 | 4.982 |
| ATOM | 15 | P18 | URI | 1 | −0.885 | −7.625 | −2.752 | −0.25 | +0.38 | +1.210 | 4.982 |
| ATOM | 16 | O19 | URI | 1 | −0.820 | −8.905 | −1.991 | +0.04 | −0.28 | −0.850 | 4.982 |
| ATOM | 17 | O20 | URI | 1 | 0.355 | −7.150 | −3.446 | +0.07 | −0.31 | −0.850 | 4.982 |
| ATOM | 18 | O21 | URI | 1 | −1.614 | −6.510 | −1.876 | +0.18 | −0.11 | −0.510 | 4.982 |
| ATOM | 19 | P22 | URI | 1 | −1.821 | −6.319 | −0.302 | −0.09 | +0.13 | +1.210 | 4.982 |
| ATOM | 20 | O25 | URI | 1 | −1.310 | −7.440 | 0.526 | +0.18 | −0.12 | −0.850 | 4.982 |
| ATOM | 21 | O24 | URI | 1 | −1.289 | −4.946 | −0.114 | +0.19 | −0.05 | −0.850 | 4.982 |
| ATOM | 22 | O23 | URI | 1 | −3.421 | −6.370 | −0.091 | +0.19 | −0.01 | −0.368 | 4.982 |
| ATOM | 23 | C37 | URI | 1 | −3.743 | −4.998 | −0.027 | −0.25 | −0.04 | +0.227 | 4.982 |
| ATOM | 24 | C45 | URI | 1 | −5.215 | −4.788 | 0.446 | −0.41 | −0.08 | +0.211 | 4.982 |
| ATOM | 25 | N41 | URI | 1 | −5.430 | −5.513 | 1.729 | −0.17 | +0.17 | −0.650 | 4.982 |
| ATOM | 26 | H41 | URI | 1 | −5.274 | −6.520 | 1.686 | +0.11 | −0.05 | +0.440 | 4.982 |
| ATOM | 27 | C49 | URI | 1 | −5.715 | −4.900 | 2.883 | −0.49 | −0.11 | +0.396 | 4.982 |
| ATOM | 28 | O38 | URI | 1 | −5.892 | −3.686 | 2.993 | −0.24 | +0.18 | −0.396 | 4.982 |
| ATOM | 29 | C50 | URI | 1 | −5.813 | −5.808 | 4.105 | −0.30 | +0.00 | +0.000 | 4.982 |
| ATOM | 30 | C44 | URI | 1 | −6.211 | −5.223 | −0.673 | −0.53 | −0.03 | +0.113 | 4.982 |
| ATOM | 31 | O47 | URI | 1 | −7.542 | −4.858 | −0.320 | −0.16 | +0.31 | −0.537 | 4.982 |
| ATOM | 32 | H47 | URI | 1 | −8.094 | −4.931 | −1.127 | −0.22 | −0.33 | +0.424 | 4.982 |
| ATOM | 33 | C43 | URI | 1 | −5.880 | −4.582 | −2.045 | −0.65 | −0.03 | +0.113 | 4.982 |
| ATOM | 34 | O48 | URI | 1 | −6.735 | −5.144 | −3.034 | −0.25 | +0.13 | −0.537 | 4.982 |
| ATOM | 35 | H48 | URI | 1 | −6.850 | −4.433 | −3.695 | −0.24 | −0.19 | +0.424 | 4.982 |
| ATOM | 36 | C46 | URI | 1 | −4.390 | −4.815 | −2.420 | −0.48 | −0.01 | +0.113 | 4.982 |
| ATOM | 37 | C42 | URI | 1 | −3.957 | −4.074 | −3.695 | −0.53 | +0.00 | +0.113 | 4.982 |
| ATOM | 38 | O39 | URI | 1 | −3.468 | −5.009 | −4.651 | −0.01 | −0.08 | −0.537 | 4.982 |
| ATOM | 39 | H39 | URI | 1 | −2.808 | −5.583 | −4.200 | +0.08 | +0.09 | +0.424 | 4.982 |
| ATOM | 40 | O40 | URI | 1 | −3.534 | −4.369 | −1.326 | +0.08 | +0.05 | −0.227 | 4.982 |
| ATOM | 41 | C12 | URI | 1 | −1.177 | −9.953 | −6.735 | −0.60 | +0.07 | +0.113 | 4.982 |
| ATOM | 42 | O15 | URI | 1 | 0.121 | −10.225 | −7.275 | −0.30 | −0.44 | −0.537 | 4.982 |
| ATOM | 43 | H15 | URI | 1 | 0.298 | −11.159 | −7.055 | +0.02 | +0.58 | +0.424 | 4.982 |
| ATOM | 44 | C10 | URI | 1 | −1.810 | −11.316 | −6.373 | −0.62 | +0.09 | +0.113 | 4.982 |
| ATOM | 45 | O11 | URI | 1 | −1.387 | −12.301 | −7.287 | +0.00 | −0.33 | −0.537 | 4.982 |
| ATOM | 46 | H11 | URI | 1 | −2.218 | −12.696 | −7.622 | −0.24 | +0.07 | +0.424 | 4.982 |
| TER | | | | | | | | | | | |
| ENDMDL | | | | | | | | | | | |
| MODEL | 17 | | | | | | | | | | |
| USER | Run = 17 | | | | | | | | | | |
| USER | Cluster Rank = 31 | | | | | | | | | | |
| USER | Number of conformations in this cluster = 1 | | | | | | | | | | |
| USER | | | | | | | | | | | |
| USER | RMSD from reference structure = 3.099 A | | | | | | | | | | |
| USER | | | | | | | | | | | |
| USER | Estimated Free Energy of Binding = −6.05 kcal/mol [= (1) + (3)] | | | | | | | | | | |
| USER | Estimated Inhibition Constant, Ki = +3.68e−05 [Temperature = 298.15 K] | | | | | | | | | | |
| USER | | | | | | | | | | | |
| USER | Final Docked Energy = −7.81 kcal/mol [= (1) + (2)] | | | | | | | | | | |
| USER | | | | | | | | | | | |
| USER | (1) Final Intermolecular Energy = −10.72 kcal/mol | | | | | | | | | | |
| USER | (2) Final Internal Energy of Ligand = +2.91 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dPf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 0.807588 −4.565979 −1.554939 | | | | | | | | | | |
| USER | NEWDPF quat0 0.981366 0.148714 0.121673 21.768899 | | | | | | | | | | |
| USER | NEWDPF ndihe 15 | | | | | | | | | | |
| USER | NEWDPF dihe0 114.99 35.53 175.70 59.08 91.17 −171.09 −41.57 32.14 | | | | | | | | | | |
| | 167.42 137.89 −64.19 −49.43 −5.37 7.19 −25.98 | | | | | | | | | | |
| USER | | | | | | | | | | | |
| USER | | | | | x | y | z | vdW | Elec | q | RMS |
| ATOM | 1 | C2 | URI | 1 | −1.601 | −11.413 | −5.813 | −0.61 | +0.24 | +0.296 | 3.099 |
| ATOM | 2 | N3 | URI | 1 | −1.441 | −12.764 | −5.632 | −0.38 | −0.21 | −0.292 | 3.099 |
| ATOM | 3 | C4 | URI | 1 | −1.209 | −13.412 | −4.418 | −0.75 | +0.24 | +0.346 | 3.099 |
| ATOM | 4 | C5 | URI | 1 | −1.124 | −12.543 | −3.287 | −0.61 | +0.00 | +0.000 | 3.099 |
| ATOM | 5 | C6 | URI | 1 | −1.284 | −11.222 | −3.453 | −0.29 | −0.03 | −0.050 | 3.099 |
| ATOM | 6 | N1 | URI | 1 | −1.503 | −10.634 | −4.694 | −0.28 | +0.03 | +0.039 | 3.099 |
| ATOM | 7 | O8 | URI | 1 | −1.081 | −14.636 | −4.429 | −0.43 | −0.38 | −0.396 | 3.099 |
| ATOM | 8 | O7 | URI | 1 | −1.808 | −10.929 | −6.926 | −0.15 | −0.29 | −0.396 | 3.099 |
| ATOM | 9 | H3 | URI | 1 | −1.497 | −13.340 | −6.452 | −0.19 | +0.04 | +0.393 | 3.099 |
| ATOM | 10 | C9 | URI | 1 | −1.649 | −9.179 | −4.819 | −0.35 | +0.10 | +0.174 | 3.099 |
| ATOM | 11 | O14 | URI | 1 | −0.765 | −8.605 | −3.863 | +0.08 | −0.10 | −0.227 | 3.099 |
| ATOM | 12 | C13 | URI | 1 | −1.209 | −7.257 | −3.549 | −0.24 | +0.04 | +0.113 | 3.099 |
| ATOM | 13 | C16 | URI | 1 | −1.109 | −7.051 | −2.037 | −0.18 | +0.03 | +0.113 | 3.099 |
| ATOM | 14 | O17 | URI | 1 | −1.657 | −5.782 | −1.736 | +0.18 | −0.07 | −0.368 | 3.099 |
| ATOM | 15 | P18 | URI | 1 | −1.415 | −4.747 | −0.569 | −0.13 | +0.08 | +1.210 | 3.099 |
| ATOM | 16 | O19 | URI | 1 | −2.472 | −3.701 | −0.662 | +0.10 | +0.20 | −0.850 | 3.099 |
| ATOM | 17 | O20 | URI | 1 | −1.279 | −5.505 | 0.716 | +0.20 | −0.04 | −0.850 | 3.099 |
| ATOM | 18 | O21 | URI | 1 | −0.041 | −4.180 | −1.145 | +0.18 | −0.14 | −0.510 | 3.099 |
| ATOM | 19 | P22 | URI | 1 | 0.315 | −2.988 | −2.149 | −0.22 | +0.86 | +1.210 | 3.099 |
| ATOM | 20 | O25 | URI | 1 | −0.511 | −2.955 | −3.382 | −0.10 | −1.94 | −0.850 | 3.099 |
| ATOM | 21 | O24 | URI | 1 | 0.319 | −1.830 | −1.220 | +0.13 | −0.27 | −0.850 | 3.099 |
| ATOM | 22 | O23 | URI | 1 | 1.802 | −3.329 | −2.679 | +0.11 | −0.26 | −0.368 | 3.099 |
| ATOM | 23 | C37 | URI | 1 | 1.628 | −4.629 | −3.196 | −0.25 | +0.14 | +0.227 | 3.099 |
| ATOM | 24 | C45 | URI | 1 | 2.706 | −5.606 | −2.632 | −0.40 | +0.14 | +0.211 | 3.099 |
| ATOM | 25 | N41 | URI | 1 | 2.697 | −5.547 | −1.144 | −0.25 | −0.37 | −0.650 | 3.099 |
| ATOM | 26 | H41 | URI | 1 | 2.996 | −4.657 | −0.747 | +0.09 | +0.26 | +0.440 | 3.099 |
| ATOM | 27 | C49 | URI | 1 | 2.248 | −6.540 | −0.370 | −0.41 | +0.15 | +0.396 | 3.099 |
| ATOM | 28 | O38 | URI | 1 | 1.856 | −7.626 | −0.800 | +0.05 | −0.08 | −0.396 | 3.099 |
| ATOM | 29 | C50 | URI | 1 | 2.238 | −6.256 | 1.129 | −0.31 | +0.00 | +0.000 | 3.099 |
| ATOM | 30 | C44 | URI | 1 | 4.103 | −5.282 | −3.246 | −0.57 | +0.13 | +0.113 | 3.099 |
| ATOM | 31 | O47 | URI | 1 | 5.048 | −6.285 | −2.881 | −0.48 | −0.94 | −0.537 | 3.099 |
| ATOM | 32 | H47 | URI | 1 | 5.143 | −6.894 | −3.643 | +0.02 | +0.65 | +0.424 | 3.099 |
| ATOM | 33 | C43 | URI | 1 | 4.067 | −5.190 | −4.792 | −0.57 | +0.13 | +0.113 | 3.099 |
| ATOM | 34 | O48 | URI | 1 | 5.336 | −4.746 | −5.257 | +0.08 | −1.02 | −0.537 | 3.099 |
| ATOM | 35 | H48 | URI | 1 | 5.907 | −5.539 | −5.214 | +0.11 | +0.85 | +0.424 | 3.099 |
| ATOM | 36 | C46 | URI | 1 | 2.947 | −4.221 | −5.263 | −0.40 | +0.10 | +0.113 | 3.099 |
| ATOM | 37 | C42 | URI | 1 | 2.755 | −4.202 | −6.788 | −0.49 | +0.10 | +0.113 | 3.099 |
| ATOM | 38 | O39 | URI | 1 | 3.133 | −2.930 | −7.303 | −0.08 | −0.64 | −0.537 | 3.099 |
| ATOM | 39 | H39 | URI | 1 | 3.360 | −2.351 | −6.540 | +0.07 | +0.55 | +0.424 | 3.099 |
| ATOM | 40 | O40 | URI | 1 | 1.678 | −4.605 | −4.653 | +0.10 | −0.15 | −0.227 | 3.099 |
| ATOM | 41 | C12 | URI | 1 | −2.643 | −7.168 | −4.071 | −0.29 | +0.05 | +0.113 | 3.099 |
| ATOM | 42 | O15 | URI | 1 | −2.659 | −6.460 | −5.315 | −0.15 | −0.30 | −0.537 | 3.099 |
| ATOM | 43 | H15 | URI | 1 | −1.842 | −5.928 | −5.317 | −0.02 | −0.01 | +0.424 | 3.099 |
| ATOM | 44 | C10 | URI | 1 | −3.012 | −8.636 | −4.383 | −0.31 | +0.07 | +0.113 | 3.099 |
| ATOM | 45 | O11 | URI | 1 | −3.929 | −8.685 | −5.452 | −0.12 | −0.61 | −0.537 | 3.099 |
| ATOM | 46 | H11 | URI | 1 | −3.446 | −9.149 | −6.167 | +0.08 | +0.39 | +0.424 | 3.099 |
| TER | | | | | | | | | | | |
| ENDMDL | | | | | | | | | | | |
| MODEL | 6 | | | | | | | | | | |
| USER | Run = 6 | | | | | | | | | | |
| USER | Cluster Rank = 32 | | | | | | | | | | |
| USER | Number of conformations in this cluster = 1 | | | | | | | | | | |
| USER | | | | | | | | | | | |
| USER | RMSD from reference structure = 3.037 A | | | | | | | | | | |
| USER | | | | | | | | | | | |
| USER | Estimated Free Energy of Binding = −5.88 kcal/mol [= (1) + (3)] | | | | | | | | | | |
| USER | Estimated Inhibition Constant, Ki = +4.89e−05 [Temperature = 298.15 K] | | | | | | | | | | |
| USER | | | | | | | | | | | |
| USER | Final Docked Energy = −7.75 kcal/mol [= (1) + (2)] | | | | | | | | | | |
| USER | | | | | | | | | | | |
| USER | (1) Final Intermolecular Energy = −10.55 kcal/mol | | | | | | | | | | |
| USER | (2) Final Internal Energy of Ligand = +2.80 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 5.209304 −5.097529 −5.554239 | | | | | | | | | | |
| USER | NEWDPF quat0 0.809619 −0.115982 0.575383 −127.490564 | | | | | | | | | | |
| USER | NEWDPF ndihe 15 | | | | | | | | | | |
| USER | NEWDPF dihe0 −103.43 4.76 139.10 52.38 −43.19 151.82 −94.84 −10.89 −43.30 | | | | | | | | | | |
| | 112.06 −94.73 −64.15 139.05 −77.82 36.39 | | | | | | | | | | |
| USER | | | | | | | | | | | |
| USER | | | | | x | y | z | vdW | Elec | q | RMS |
| ATOM | 1 | C2 | URI | 1 | 0.291 | 0.204 | −1.250 | −0.33 | +0.03 | +0.296 | 3.037 |
| ATOM | 2 | N3 | URI | 1 | 0.522 | 1.217 | −0.354 | −0.21 | +0.06 | −0.292 | 3.037 |
| ATOM | 3 | C4 | URI | 1 | 1.693 | 1.969 | −0.242 | −0.46 | −0.08 | +0.346 | 3.037 |
| ATOM | 4 | C5 | URI | 1 | 2.725 | 1.602 | −1.160 | −0.57 | +0.00 | +0.000 | 3.037 |
| ATOM | 5 | C6 | URI | 1 | 2.507 | 0.615 | −2.042 | −0.51 | +0.00 | −0.050 | 3.037 |
| ATOM | 6 | N1 | URI | 1 | 1.319 | −0.105 | −2.097 | −0.25 | +0.01 | +0.039 | 3.037 |
| ATOM | 7 | O8 | URI | 1 | 1.741 | 2.842 | 0.624 | +0.01 | +0.22 | −0.396 | 3.037 |
| ATOM | 8 | O7 | URI | 1 | −0.780 | −0.404 | −1.279 | +0.01 | −0.03 | −0.396 | 3.037 |
| ATOM | 9 | H3 | URI | 1 | −0.223 | 1.438 | 0.281 | +0.10 | −0.16 | +0.393 | 3.037 |
| ATOM | 10 | C9 | URI | 1 | 1.144 | −1.189 | −3.070 | −0.40 | +0.19 | +0.174 | 3.037 |
| ATOM | 11 | O14 | URI | 1 | 1.416 | −2.400 | −2.374 | +0.08 | −0.16 | −0.227 | 3.037 |
| ATOM | 12 | C13 | URI | 1 | 1.779 | −3.432 | −3.330 | −0.29 | +0.10 | +0.113 | 3.037 |
| ATOM | 13 | C16 | URI | 1 | 2.973 | −4.210 | −2.776 | −0.22 | +0.09 | +0.113 | 3.037 |
| ATOM | 14 | O17 | URI | 1 | 2.483 | −5.414 | −2.216 | +0.06 | −0.22 | −0.368 | 3.037 |
| ATOM | 15 | P18 | URI | 1 | 1.033 | −5.958 | −1.915 | −0.25 | +0.45 | +1.210 | 3.037 |
| ATOM | 16 | O19 | URI | 1 | 1.165 | −7.355 | −1.414 | −0.02 | −0.22 | −0.850 | 3.037 |
| ATOM | 17 | O20 | URI | 1 | 0.321 | −4.960 | −1.054 | +0.18 | −0.24 | −0.850 | 3.037 |
| ATOM | 18 | O21 | URI | 1 | 0.552 | −5.972 | −3.436 | +0.12 | −0.20 | −0.510 | 3.037 |
| ATOM | 19 | P22 | URI | 1 | 1.095 | −6.709 | −4.747 | −0.35 | +0.49 | +1.210 | 3.037 |
| ATOM | 20 | O25 | URI | 1 | 2.363 | −6.155 | −5.284 | +0.05 | −0.51 | −0.850 | 3.037 |
| ATOM | 21 | O24 | URI | 1 | 1.019 | −8.130 | −4.322 | +0.42 | −0.43 | −0.850 | 3.037 |
| ATOM | 22 | O23 | URI | 1 | 0.003 | −6.372 | −5.888 | +0.09 | −0.02 | −0.368 | 3.037 |
| ATOM | 23 | C37 | URI | 1 | 0.175 | −7.454 | −6.775 | −0.50 | +0.07 | +0.227 | 3.037 |
| ATOM | 24 | C45 | URI | 1 | −0.651 | −7.247 | −8.082 | −0.29 | +0.05 | +0.211 | 3.037 |
| ATOM | 25 | N41 | URI | 1 | −0.311 | −5.926 | −8.678 | −0.16 | +0.06 | −0.650 | 3.037 |
| ATOM | 26 | H41 | URI | 1 | −1.094 | −5.278 | −8.764 | −0.26 | −0.25 | +0.440 | 3.037 |
| ATOM | 27 | C49 | URI | 1 | 0.934 | −5.560 | −8.997 | −0.68 | +0.12 | +0.396 | 3.037 |
| ATOM | 28 | O38 | URI | 1 | 1.917 | −6.296 | −8.892 | −0.14 | −0.25 | −0.396 | 3.037 |
| ATOM | 29 | C50 | URI | 1 | 1.091 | −4.133 | −9.514 | −0.78 | +0.00 | +0.000 | 3.037 |
| ATOM | 30 | C44 | URI | 1 | −2.175 | −7.409 | −7.789 | −0.62 | +0.05 | +0.113 | 3.037 |
| ATOM | 31 | O47 | URI | 1 | −2.915 | −7.408 | −9.007 | −0.14 | −0.30 | −0.537 | 3.037 |
| ATOM | 32 | H47 | URI | 1 | −3.521 | −6.638 | −8.985 | −0.01 | +0.36 | +0.424 | 3.037 |
| ATOM | 33 | C43 | URI | 1 | −2.499 | −8.718 | −7.026 | −0.56 | +0.08 | +0.113 | 3.037 |
| ATOM | 34 | O48 | URI | 1 | −3.876 | −8.714 | −6.669 | −0.19 | −0.62 | −0.537 | 3.037 |
| ATOM | 35 | H48 | URI | 1 | −4.343 | −8.454 | −7.487 | +0.05 | +0.45 | +0.424 | 3.037 |
| ATOM | 36 | C46 | URI | 1 | −1.610 | −8.855 | −5.759 | −0.41 | +0.06 | +0.113 | 3.037 |
| ATOM | 37 | C42 | URI | 1 | −1.771 | −10.204 | −5.041 | −0.44 | +0.08 | +0.113 | 3.037 |
| ATOM | 38 | O39 | URI | 1 | −1.529 | −10.039 | −3.648 | +0.06 | −0.30 | −0.537 | 3.037 |
| ATOM | 39 | H39 | URI | 1 | −0.761 | −9.432 | −3.545 | +0.08 | +0.22 | +0.424 | 3.037 |
| ATOM | 40 | O40 | URI | 1 | −0.206 | −8.704 | −6.127 | −0.01 | −0.11 | −0.227 | 3.037 |
| ATOM | 41 | C12 | URI | 1 | 2.098 | −2.691 | −4.628 | −0.43 | +0.13 | +0.113 | 3.037 |
| ATOM | 42 | O15 | URI | 1 | 0.992 | −2.799 | −5.530 | −0.33 | −0.80 | −0.537 | 3.037 |
| ATOM | 43 | H15 | URI | 1 | 0.562 | −3.645 | −5.306 | −0.03 | +0.39 | +0.424 | 3.037 |
| ATOM | 44 | C10 | URI | 1 | 2.184 | −1.209 | −4.194 | −0.60 | +0.13 | +0.113 | 3.037 |
| ATOM | 45 | O11 | URI | 1 | 1.782 | −0.374 | −5.255 | −0.22 | −1.10 | −0.537 | 3.037 |
| ATOM | 46 | H11 | URI | 1 | 1.854 | −0.939 | −6.052 | +0.04 | +0.83 | +0.424 | 3.037 |
| TER | | | | | | | | | | | |
| ENDMDL | | | | | | | | | | | |
| MODEL | 49 | | | | | | | | | | |
| USER | Run = 49 | | | | | | | | | | |
| USER | Cluster Rank = 33 | | | | | | | | | | |
| USER | Number of conformations in this cluster = 1 | | | | | | | | | | |
| USER | | | | | | | | | | | |
| USER | RMSD from reference structure = 4.285 A | | | | | | | | | | |
| USER | | | | | | | | | | | |
| USER | Estimated Free Energy of Binding = −3.95 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −7.72 kcal/mol [= (1) + (2)] | | | | | | | | | | |
| USER | | | | | | | | | | | |
| USER | (1) Final Intermolecular Energy = −8.61 kcal/mol | | | | | | | | | | |
| USER | (2) Final Internal Energy of Ligand = +0.89 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −2.817486 −5.492072 0.550288 | | | | | | | | | | |
| USER | NEWDPF quat0 0.541074 −0.176667 −0.822209 166.340355 | | | | | | | | | | |
| USER | NEWDPF ndihe 15 | | | | | | | | | | |
| USER | NEWDPF dihe0 −63.77 −134.96 18.43 −40.73 −163.72 125.87 −40.71 0.82 | | | | | | | | | | |
| | −10.31 −109.79 −180.00 73.55 −84.35 −29.56 20.90 | | | | | | | | | | |
| USER | | | | | | | | | | | |
| USER | | | | | x | y | z | vdW | Elec | q | RMS |
| ATOM | 1 | C2 | URI | 1 | −0.346 | 2.375 | −1.127 | −0.55 | −0.06 | +0.296 | 4.285 |
| ATOM | 2 | N3 | URI | 1 | −0.997 | 3.540 | −1.445 | −0.47 | +0.07 | −0.292 | 4.285 |
| ATOM | 3 | C4 | URI | 1 | −2.319 | 3.859 | −1.132 | −0.83 | −0.10 | +0.346 | 4.285 |
| ATOM | 4 | C5 | URI | 1 | −3.026 | 2.836 | −0.426 | −0.76 | +0.00 | +0.000 | 4.285 |
| ATOM | 5 | C6 | URI | 1 | −2.397 | 1.696 | −0.107 | −0.67 | +0.02 | −0.050 | 4.285 |
| ATOM | 6 | N1 | URI | 1 | −1.077 | 1.434 | −0.457 | −0.32 | −0.01 | +0.039 | 4.285 |
| ATOM | 7 | O8 | URI | 1 | −2.756 | 4.950 | −1.498 | −0.24 | +0.09 | −0.396 | 4.285 |
| ATOM | 8 | O7 | URI | 1 | 0.830 | 2.183 | −1.441 | −0.07 | +0.06 | −0.396 | 4.285 |
| ATOM | 9 | H3 | URI | 1 | −0.470 | 4.230 | −1.950 | +0.03 | −0.06 | +0.393 | 4.285 |
| ATOM | 10 | C9 | URI | 1 | −0.446 | 0.158 | −0.100 | −0.35 | −0.04 | +0.174 | 4.285 |
| ATOM | 11 | O14 | URI | 1 | −0.841 | −0.777 | −1.097 | +0.02 | −0.01 | −0.227 | 4.285 |
| ATOM | 12 | C13 | URI | 1 | −0.718 | −2.124 | −0.566 | −0.25 | +0.00 | +0.113 | 4.285 |
| ATOM | 13 | C16 | URI | 1 | −1.952 | −2.926 | −0.982 | −0.03 | −0.02 | +0.113 | 4.285 |
| ATOM | 14 | O17 | URI | 1 | −1.558 | −4.276 | −1.143 | +0.14 | −0.03 | −0.368 | 4.285 |
| ATOM | 15 | P18 | URI | 1 | −2.103 | −5.639 | −0.566 | −0.11 | +0.07 | +1.210 | 4.285 |
| ATOM | 16 | O19 | URI | 1 | −2.314 | −5.462 | 0.899 | +0.19 | +0.03 | −0.850 | 4.285 |
| ATOM | 17 | O20 | URI | 1 | −3.266 | −6.077 | −1.401 | +0.17 | −0.07 | −0.850 | 4.285 |
| ATOM | 18 | O21 | URI | 1 | −0.763 | −6.465 | −0.823 | +0.17 | −0.10 | −0.510 | 4.285 |
| ATOM | 19 | P22 | URI | 1 | 0.433 | −6.308 | −1.873 | −0.23 | +0.37 | +1.210 | 4.285 |
| ATOM | 20 | O25 | URI | 1 | 1.241 | −5.074 | −1.706 | +0.15 | −0.35 | −0.850 | 4.285 |
| ATOM | 21 | O24 | URI | 1 | 1.075 | −7.643 | −1.768 | +0.01 | −0.22 | −0.850 | 4.285 |
| ATOM | 22 | O23 | URI | 1 | −0.298 | −6.112 | −3.300 | +0.14 | −0.12 | −0.368 | 4.285 |
| ATOM | 23 | C37 | URI | 1 | 0.237 | −7.183 | −4.043 | −0.31 | +0.08 | +0.227 | 4.285 |
| ATOM | 24 | C45 | URI | 1 | 0.075 | −6.940 | −5.576 | −0.35 | +0.05 | +0.211 | 4.285 |
| ATOM | 25 | N41 | URI | 1 | 0.657 | −5.617 | −5.931 | −0.09 | −0.05 | −0.650 | 4.285 |
| ATOM | 26 | H41 | URI | 1 | 0.175 | −4.813 | −5.529 | −0.19 | −0.04 | +0.440 | 4.285 |
| ATOM | 27 | C49 | URI | 1 | 1.779 | −5.472 | −6.644 | −0.41 | +0.17 | +0.396 | 4.285 |
| ATOM | 28 | O38 | URI | 1 | 2.410 | −6.410 | −7.133 | −0.01 | −0.23 | −0.396 | 4.285 |
| ATOM | 29 | C50 | URI | 1 | 2.263 | −4.037 | −6.828 | −0.48 | +0.00 | +0.000 | 4.285 |
| ATOM | 30 | C44 | URI | 1 | −1.423 | −7.077 | −5.990 | −0.38 | +0.03 | +0.113 | 4.285 |
| ATOM | 31 | O47 | URI | 1 | −1.549 | −7.043 | −7.409 | −0.09 | −0.10 | −0.537 | 4.285 |
| ATOM | 32 | H47 | URI | 1 | −1.482 | −6.106 | −7.690 | −0.24 | −0.24 | +0.424 | 4.285 |
| ATOM | 33 | C43 | URI | 1 | −2.064 | −8.392 | −5.477 | −0.37 | +0.07 | +0.113 | 4.285 |
| ATOM | 34 | O48 | URI | 1 | −3.457 | −8.368 | −5.765 | −0.20 | −0.62 | −0.537 | 4.285 |
| ATOM | 35 | H48 | URI | 1 | −3.869 | −7.991 | −4.962 | +0.08 | +0.49 | +0.424 | 4.285 |
| ATOM | 36 | C46 | URI | 1 | −1.827 | −8.565 | −3.951 | −0.27 | +0.05 | +0.113 | 4.285 |
| ATOM | 37 | C42 | URI | 1 | −2.303 | −9.922 | −3.409 | −0.29 | +0.06 | +0.113 | 4.285 |
| ATOM | 38 | O39 | URI | 1 | −2.128 | −9.963 | −1.997 | +0.11 | −0.22 | −0.537 | 4.285 |
| ATOM | 39 | H39 | URI | 1 | −2.149 | −10.907 | −1.717 | +0.10 | +0.19 | +0.424 | 4.285 |
| ATOM | 40 | O40 | URI | 1 | −0.404 | −8.436 | −3.658 | +0.11 | −0.10 | −0.227 | 4.285 |
| ATOM | 41 | C12 | URI | 1 | −0.596 | −1.957 | 0.948 | −0.21 | −0.02 | +0.113 | 4.285 |
| ATOM | 42 | O15 | URI | 1 | 0.773 | −2.101 | 1.339 | +0.19 | +0.03 | −0.537 | 4.285 |
| ATOM | 43 | H15 | URI | 1 | 0.779 | −2.836 | 1.981 | +0.12 | −0.01 | +0.424 | 4.285 |
| ATOM | 44 | C10 | URI | 1 | −0.979 | −0.478 | 1.186 | −0.35 | −0.06 | +0.113 | 4.285 |
| ATOM | 45 | O11 | URI | 1 | −0.291 | 0.022 | 2.308 | +0.09 | +0.40 | −0.537 | 4.285 |
| ATOM | 46 | H11 | URI | 1 | −0.955 | 0.567 | 2.779 | −0.12 | −0.66 | +0.424 | 4.285 |
| TER | | | | | | | | | | | |
| ENDMDL | | | | | | | | | | | |
| MODEL | 26 | | | | | | | | | | |
| USER | Run = 26 | | | | | | | | | | |
| USER | Cluster Rank = 34 | | | | | | | | | | |
| USER | Number of conformations in this cluster = 1 | | | | | | | | | | |
| USER | | | | | | | | | | | |
| USER | RMSD from reference structure = 4.457 A | | | | | | | | | | |
| USER | | | | | | | | | | | |
| USER | Estimated Free Energy of Binding = −5.16 kcal/mol [= (1) + (3)] | | | | | | | | | | |
| USER | Estimated Inhibition Constant, Ki = +1.65e−04 [Temperature = 298.15 K] | | | | | | | | | | |
| USER | | | | | | | | | | | |
| USER | Final Docked Energy = −7.72 kcal/mol [= (1) + (2)] | | | | | | | | | | |
| USER | | | | | | | | | | | |
| USER | (1) Final Intermolecular Energy = −9.83 kcal/mol | | | | | | | | | | |
| USER | (2) Final Internal Energy of Ligand = +2.11 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −0.826106 −4.019805 1.292108 | | | | | | | | | | |
| USER | NEWDPF quat0 0.002668 −0.689388 −0.724387 −158.242020 | | | | | | | | | | |
| USER | NEWDPF ndihe 15 | | | | | | | | | | |
| USER | NEWDPF dihe0 53.79 −108.21 5.32 −21.14 161.37 −147.95 124.53 15.41 33.71 | | | | | | | | | | |
| | −172.82 −69.36 167.93 40.34 −100.82 10.63 | | | | | | | | | | |
| USER | | | | | | | | | | | |
| USER | | | | | x | y | z | vdW | Elec | q | RMS |
| ATOM | 1 | C2 | URI | 1 | 3.297 | −6.180 | −5.718 | −0.54 | +0.24 | +0.296 | 4.457 |
| ATOM | 2 | N3 | URI | 1 | 3.720 | −5.502 | −6.833 | −0.34 | −0.31 | −0.292 | 4.457 |
| ATOM | 3 | C4 | URI | 1 | 3.984 | −4.133 | −6.916 | −0.50 | +0.50 | +0.346 | 4.457 |
| ATOM | 4 | C5 | URI | 1 | 3.765 | −3.411 | −5.702 | −0.55 | +0.00 | +0.000 | 4.457 |
| ATOM | 5 | C6 | URI | 1 | 3.356 | −4.067 | −4.606 | −0.43 | −0.05 | −0.050 | 4.457 |
| ATOM | 6 | N1 | URI | 1 | 3.099 | −5.434 | −4.590 | −0.24 | +0.03 | +0.039 | 4.457 |
| ATOM | 7 | O8 | URI | 1 | 4.351 | −3.677 | −7.998 | −0.31 | −0.73 | −0.396 | 4.457 |
| ATOM | 8 | O7 | URI | 1 | 3.094 | −7.395 | −5.738 | −0.12 | −0.25 | −0.396 | 4.457 |
| ATOM | 9 | H3 | URI | 1 | 3.852 | −6.041 | −7.669 | +0.06 | +0.39 | +0.393 | 4.457 |
| ATOM | 10 | C9 | URI | 1 | 2.632 | −6.095 | −3.366 | −0.41 | +0.11 | +0.174 | 4.457 |
| ATOM | 11 | O14 | URI | 1 | 1.283 | −5.684 | −3.179 | +0.12 | −0.11 | −0.227 | 4.457 |
| ATOM | 12 | C13 | URI | 1 | 0.923 | −5.844 | −1.780 | −0.23 | +0.04 | +0.113 | 4.457 |
| ATOM | 13 | C16 | URI | 1 | 0.136 | −4.611 | −1.336 | −0.14 | +0.03 | +0.113 | 4.457 |
| ATOM | 14 | O17 | URI | 1 | −1.180 | −4.732 | −1.839 | +0.16 | −0.08 | −0.368 | 4.457 |
| ATOM | 15 | P18 | URI | 1 | −1.972 | −3.998 | −2.990 | −0.36 | +0.38 | +1.210 | 4.457 |
| ATOM | 16 | O19 | URI | 1 | −1.101 | −3.974 | −4.199 | −0.20 | −0.65 | −0.850 | 4.457 |
| ATOM | 17 | O20 | URI | 1 | −3.328 | −4.623 | −3.106 | −0.02 | −0.04 | −0.850 | 4.457 |
| ATOM | 18 | O21 | URI | 1 | −1.968 | −2.564 | −2.293 | +0.00 | −0.09 | −0.510 | 4.457 |
| ATOM | 19 | P22 | URI | 1 | −0.903 | −1.372 | −2.249 | −0.39 | +0.84 | +1.210 | 4.457 |
| ATOM | 20 | O25 | URI | 1 | 0.319 | −1.610 | −3.057 | −0.26 | −1.57 | −0.850 | 4.457 |
| ATOM | 21 | O24 | URI | 1 | −1.775 | −0.206 | −2.537 | +0.07 | −0.21 | −0.850 | 4.457 |
| ATOM | 22 | O23 | URI | 1 | −0.371 | −1.353 | −0.725 | +0.11 | −0.02 | −0.368 | 4.457 |
| ATOM | 23 | C37 | URI | 1 | −1.431 | −0.701 | −0.062 | −0.43 | −0.08 | +0.227 | 4.457 |
| ATOM | 24 | C45 | URI | 1 | −0.902 | 0.179 | 1.113 | −0.38 | −0.12 | +0.211 | 4.457 |
| ATOM | 25 | N41 | URI | 1 | 0.146 | 1.106 | 0.603 | −0.17 | +0.25 | −0.650 | 4.457 |
| ATOM | 26 | H41 | URI | 1 | −0.173 | 2.058 | 0.421 | +0.10 | −0.23 | +0.440 | 4.457 |
| ATOM | 27 | C49 | URI | 1 | 1.432 | 0.766 | 0.475 | −0.25 | −0.08 | +0.396 | 4.457 |
| ATOM | 28 | O38 | URI | 1 | 1.872 | −0.362 | 0.702 | +0.16 | +0.03 | −0.396 | 4.457 |
| ATOM | 29 | C50 | URI | 1 | 2.368 | 1.882 | 0.020 | −0.44 | +0.00 | +0.000 | 4.457 |
| ATOM | 30 | C44 | URI | 1 | −0.399 | −0.724 | 2.282 | −0.25 | −0.07 | +0.113 | 4.457 |
| ATOM | 31 | O47 | URI | 1 | −0.093 | 0.071 | 3.424 | +0.13 | +0.52 | −0.537 | 4.457 |
| ATOM | 32 | H47 | URI | 1 | −0.775 | 0.773 | 3.488 | −0.16 | −0.84 | +0.424 | 4.457 |
| ATOM | 33 | C43 | URI | 1 | −1.442 | −1.792 | 2.699 | −0.33 | −0.07 | +0.113 | 4.457 |
| ATOM | 34 | O48 | URI | 1 | −0.851 | −2.662 | 3.657 | +0.11 | +0.17 | −0.537 | 4.457 |
| ATOM | 35 | H48 | URI | 1 | −0.142 | −3.124 | 3.168 | +0.11 | −0.07 | +0.424 | 4.457 |
| ATOM | 36 | C46 | URI | 1 | −1.934 | −2.598 | 1.464 | −0.33 | −0.05 | +0.113 | 4.457 |
| ATOM | 37 | C42 | URI | 1 | −3.081 | −3.568 | 1.785 | −0.31 | −0.06 | +0.113 | 4.457 |
| ATOM | 38 | O39 | URI | 1 | −2.549 | −4.835 | 2.156 | +0.17 | +0.08 | −0.537 | 4.457 |
| ATOM | 39 | H39 | URI | 1 | −3.304 | −5.457 | 2.271 | +0.11 | −0.05 | +0.424 | 4.457 |
| ATOM | 40 | O40 | URI | 1 | −2.396 | −1.677 | 0.431 | +0.00 | +0.14 | −0.227 | 4.457 |
| ATOM | 41 | C12 | URI | 1 | 2.247 | −6.010 | −1.035 | −0.38 | +0.05 | +0.113 | 4.457 |
| ATOM | 42 | O15 | URI | 1 | 2.459 | −7.392 | −0.730 | +0.02 | −0.15 | −0.537 | 4.457 |
| ATOM | 43 | H15 | URI | 1 | 2.234 | −7.483 | 0.214 | +0.05 | +0.10 | +0.424 | 4.457 |
| ATOM | 44 | C10 | URI | 1 | 3.311 | −5.611 | −2.083 | −0.51 | +0.09 | +0.113 | 4.457 |
| ATOM | 45 | O11 | URI | 1 | 4.504 | −6.325 | −1.854 | −0.51 | −0.76 | −0.537 | 4.457 |
| ATOM | 46 | H11 | URI | 1 | 5.143 | −5.945 | −2.491 | +0.01 | +0.89 | +0.424 | 4.457 |
| TER | | | | | | | | | | | |
| ENDMDL | | | | | | | | | | | |
| MODEL | 47 | | | | | | | | | | |
| USER | Run = 47 | | | | | | | | | | |
| USER | Cluster Rank = 35 | | | | | | | | | | |
| USER | Number of conformations in this cluster = 1 | | | | | | | | | | |
| USER | | | | | | | | | | | |
| USER | RMSD from reference structure = 3.633 A | | | | | | | | | | |
| USER | | | | | | | | | | | |
| USER | Estimated Free Energy of Binding = −5.70 kcal/mol [= (1) + (3)] | | | | | | | | | | |
| USER | Estimated Inhibition Constant, Ki = +6.62e−05 [Temperature = 298.15 K] | | | | | | | | | | |
| USER | | | | | | | | | | | |
| USER | Final Docked Energy = −7.71 kcal/mol [= (1) + (2)] | | | | | | | | | | |
| USER | | | | | | | | | | | |
| USER | (1) Final Intermolecular Energy = −10.37 kcal/mol | | | | | | | | | | |
| USER | (2) Final Internal Energy of Ligand = +2.66 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −3.017886 −8.050104 0.277154 | | | | | | | | | | |
| USER | NEWDPF quat0 −0.528134 0.629479 0.569938 −179.977435 | | | | | | | | | | |
| USER | NEWDPF ndihe 15 | | | | | | | | | | |
| USER | NEWDPF dihe0 −141.10 −155.95 −178.38 168.74 127.12 142.94 125.20 −12.71 | | | | | | | | | | |
| | −1.74 −116.04 −175.76 88.32 62.26 −124.91 −10.70 | | | | | | | | | | |
| USER | | | | | | | | | | | |
| USER | | | | | x | y | z | vdW | Elec | q | RMS |
| ATOM | 1 | C2 | URI | 1 | 4.317 | −5.873 | −3.225 | −0.66 | +0.36 | +0.296 | 3.633 |
| ATOM | 2 | N3 | URI | 1 | 4.676 | −5.196 | −4.363 | −0.44 | −0.39 | −0.292 | 3.633 |
| ATOM | 3 | C4 | URI | 1 | 3.896 | −4.264 | −5.050 | −0.53 | +0.40 | +0.346 | 3.633 |
| ATOM | 4 | C5 | URI | 1 | 2.604 | −4.038 | −4.484 | −0.35 | +0.00 | +0.000 | 3.633 |
| ATOM | 5 | C6 | URI | 1 | 2.246 | −4.692 | −3.368 | −0.30 | −0.03 | −0.050 | 3.633 |
| ATOM | 6 | N1 | URI | 1 | 3.067 | −5.618 | −2.735 | −0.28 | +0.03 | +0.039 | 3.633 |
| ATOM | 7 | O8 | URI | 1 | 4.366 | −3.749 | −6.065 | −0.08 | −0.66 | −0.396 | 3.633 |
| ATOM | 8 | O7 | URI | 1 | 5.077 | −6.676 | −2.683 | −0.53 | −0.72 | −0.396 | 3.633 |
| ATOM | 9 | H3 | URI | 1 | 5.588 | −5.390 | −4.737 | +0.06 | +0.69 | +0.393 | 3.633 |
| ATOM | 10 | C9 | URI | 1 | 2.613 | −6.320 | −1.528 | −0.44 | +0.09 | +0.174 | 3.633 |
| ATOM | 11 | O14 | URI | 1 | 1.334 | −6.861 | −1.837 | +0.01 | −0.08 | −0.227 | 3.633 |
| ATOM | 12 | C13 | URI | 1 | 0.605 | −7.098 | −0.603 | −0.30 | +0.02 | +0.113 | 3.633 |
| ATOM | 13 | C16 | URI | 1 | −0.839 | −6.631 | −0.793 | −0.14 | +0.02 | +0.113 | 3.633 |
| ATOM | 14 | O17 | URI | 1 | −1.348 | −7.257 | −1.956 | +0.16 | −0.09 | −0.368 | 3.633 |
| ATOM | 15 | P18 | URI | 1 | −1.296 | −6.898 | −3.491 | −0.22 | +0.37 | +1.210 | 3.633 |
| ATOM | 16 | O19 | URI | 1 | −1.288 | −5.412 | −3.611 | +0.09 | −0.23 | −0.850 | 3.633 |
| ATOM | 17 | O20 | URI | 1 | −0.176 | −7.666 | −4.121 | +0.07 | −0.32 | −0.850 | 3.633 |
| ATOM | 18 | O21 | URI | 1 | −2.749 | −7.459 | −3.832 | +0.10 | −0.24 | −0.510 | 3.633 |
| ATOM | 19 | P22 | URI | 1 | −3.704 | −8.526 | −3.119 | −0.24 | +0.65 | +1.210 | 3.633 |
| ATOM | 20 | O25 | URI | 1 | −4.074 | −8.184 | −1.723 | +0.18 | −0.27 | −0.850 | 3.633 |
| ATOM | 21 | O24 | URI | 1 | −4.757 | −8.694 | −4.152 | −0.15 | −0.96 | −0.850 | 3.633 |
| ATOM | 22 | O23 | URI | 1 | −2.797 | −9.854 | −2.974 | +0.13 | −0.19 | −0.368 | 3.633 |
| ATOM | 23 | C37 | URI | 1 | −3.462 | −10.748 | −3.838 | −0.31 | +0.18 | +0.227 | 3.633 |
| ATOM | 24 | C45 | URI | 1 | −2.844 | −12.178 | −3.751 | −0.24 | +0.17 | +0.211 | 3.633 |
| ATOM | 25 | N41 | URI | 1 | −2.826 | −12.623 | −2.331 | −0.19 | −0.36 | −0.650 | 3.633 |
| ATOM | 26 | H41 | URI | 1 | −3.577 | −13.261 | −2.070 | +0.11 | +0.23 | +0.440 | 3.633 |
| ATOM | 27 | C49 | URI | 1 | −1.856 | −12.299 | −1.470 | −0.41 | +0.21 | +0.396 | 3.633 |
| ATOM | 28 | O38 | URI | 1 | −0.915 | −11.550 | −1.741 | +0.00 | −0.26 | −0.396 | 3.633 |
| ATOM | 29 | C50 | URI | 1 | −1.968 | −12.925 | −0.083 | −0.20 | +0.00 | +0.000 | 3.633 |
| ATOM | 30 | C44 | URI | 1 | −1.432 | −12.198 | −4.414 | −0.61 | +0.08 | +0.113 | 3.633 |
| ATOM | 31 | O47 | URI | 1 | −0.948 | −13.535 | −4.505 | −0.28 | −0.38 | −0.537 | 3.633 |
| ATOM | 32 | H47 | URI | 1 | 0.031 | −13.494 | −4.545 | +0.02 | +0.27 | +0.424 | 3.633 |
| ATOM | 33 | C43 | URI | 1 | −1.434 | −11.586 | −5.838 | −0.64 | +0.09 | +0.113 | 3.633 |
| ATOM | 34 | O48 | URI | 1 | −0.093 | −11.505 | −6.306 | −0.38 | −0.71 | −0.537 | 3.633 |
| ATOM | 35 | H48 | URI | 1 | 0.091 | −10.547 | −6.367 | +0.04 | +0.37 | +0.424 | 3.633 |
| ATOM | 36 | C46 | URI | 1 | −2.094 | −10.179 | −5.834 | −0.49 | +0.08 | +0.113 | 3.633 |
| ATOM | 37 | C42 | URI | 1 | −2.267 | −9.581 | −7.239 | −0.58 | +0.07 | +0.113 | 3.633 |
| ATOM | 38 | O39 | URI | 1 | −2.411 | −8.168 | −7.145 | −0.17 | −0.36 | −0.537 | 3.633 |
| ATOM | 39 | H39 | URI | 1 | −2.336 | −7.919 | −6.196 | +0.09 | +0.29 | +0.424 | 3.633 |
| ATOM | 40 | O40 | URI | 1 | −3.411 | −10.256 | −5.211 | −0.22 | −0.23 | −0.227 | 3.633 |
| ATOM | 41 | C12 | URI | 1 | 1.352 | −6.310 | 0.471 | −0.26 | +0.03 | +0.113 | 3.633 |
| ATOM | 42 | O15 | URI | 1 | 2.165 | −7.199 | 1.244 | −0.01 | −0.13 | −0.537 | 3.633 |
| ATOM | 43 | H15 | URI | 1 | 3.012 | −7.255 | 0.763 | +0.05 | +0.17 | +0.424 | 3.633 |
| ATOM | 44 | C10 | URI | 1 | 2.313 | −5.408 | −0.337 | −0.31 | +0.05 | +0.113 | 3.633 |
| ATOM | 45 | O11 | URI | 1 | 3.484 | −5.164 | 0.408 | +0.02 | −0.32 | −0.537 | 3.633 |
| ATOM | 46 | H11 | URI | 1 | 4.178 | −5.022 | −0.269 | +0.06 | +0.40 | +0.424 | 3.633 |
| TER | | | | | | | | | | | |
| ENDMDL | | | | | | | | | | | |
| MODEL | 24 | | | | | | | | | | |
| USER | Run = 24 | | | | | | | | | | |
| USER | Cluster Rank = 36 | | | | | | | | | | |
| USER | Number of conformations in this cluster = 1 | | | | | | | | | | |
| USER | | | | | | | | | | | |
| USER | RMSD from reference structure = 2.636 A | | | | | | | | | | |
| USER | | | | | | | | | | | |
| USER | Estimated Free Energy of Binding = −4.28 kcal/mol [= (1) + (3)] | | | | | | | | | | |
| USER | Estimated Inhibition Constant, Ki = +7.35e−04 [Temperature = 298.15 K] | | | | | | | | | | |
| USER | | | | | | | | | | | |
| USER | Final Docked Energy = −7.38 kcal/mol [= (1) + (2)] | | | | | | | | | | |
| USER | | | | | | | | | | | |
| USER | (1) Final Intermolecular Energy = −8.94 kcal/mol | | | | | | | | | | |
| USER | (2) Final Internal Energy of Ligand = +1.57 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 −1.398304 −5.315962 0.129812 | | | | | | | | | | |
| USER | NEWDPF quat0 0.848705 0.106733 0.517984 36.219485 | | | | | | | | | | |
| USER | NEWDPF ndihe 15 | | | | | | | | | | |
| USER | NEWDPF dihe0 129.28 −60.88 −123.19 −11.57 179.97 −169.85 −58.63 27.71 | | | | | | | | | | |
| | −8.55 12.58 11.81 −179.99 141.34 −46.67 −20.41 | | | | | | | | | | |
| USER | | | | | | | | | | | |
| USER | | | | | x | y | z | vdW | Elec | q | RMS |
| ATOM | 1 | C2 | URI | 1 | −1.821 | −11.696 | −5.341 | −0.47 | +0.26 | +0.296 | 2.636 |
| ATOM | 2 | N3 | URI | 1 | −1.305 | −12.966 | −5.379 | −0.42 | −0.20 | −0.292 | 2.636 |
| ATOM | 3 | C4 | URI | 1 | −0.921 | −13.734 | −4.278 | −0.76 | +0.25 | +0.346 | 2.636 |
| ATOM | 4 | C5 | URI | 1 | −1.087 | −13.086 | −3.014 | −0.58 | +0.00 | +0.000 | 2.636 |
| ATOM | 5 | C6 | URI | 1 | −1.595 | −11.846 | −2.968 | −0.49 | −0.03 | −0.050 | 2.636 |
| ATOM | 6 | N1 | URI | 1 | −1.950 | −11.128 | −4.105 | −0.27 | +0.03 | +0.039 | 2.636 |
| ATOM | 7 | O8 | URI | 1 | −0.468 | −14.860 | −4.484 | −0.50 | −0.47 | −0.396 | 2.636 |
| ATOM | 8 | O7 | URI | 1 | −2.138 | −11.095 | −6.369 | −0.24 | −0.34 | −0.396 | 2.636 |
| ATOM | 9 | H3 | URI | 1 | −1.193 | −13.384 | −6.285 | −0.20 | +0.16 | +0.393 | 2.636 |
| ATOM | 10 | C9 | URI | 1 | −2.481 | −9.764 | −3.994 | −0.31 | +0.11 | +0.174 | 2.636 |
| ATOM | 11 | O14 | URI | 1 | −1.707 | −9.113 | −2.994 | +0.10 | −0.10 | −0.227 | 2.636 |
| ATOM | 12 | C13 | URI | 1 | −2.462 | −7.994 | −2.456 | −0.17 | +0.04 | +0.113 | 2.636 |
| ATOM | 13 | C16 | URI | 1 | −2.302 | −7.984 | −0.936 | −0.13 | +0.02 | +0.113 | 2.636 |
| ATOM | 14 | O17 | URI | 1 | −1.961 | −6.667 | −0.545 | +0.20 | −0.05 | −0.368 | 2.636 |
| ATOM | 15 | P18 | URI | 1 | −1.607 | −5.356 | −1.347 | −0.14 | +0.17 | +1.210 | 2.636 |
| ATOM | 16 | O19 | URI | 1 | −2.775 | −5.021 | −2.210 | +0.10 | −0.06 | −0.850 | 2.636 |
| ATOM | 17 | O20 | URI | 1 | −1.122 | −4.322 | −0.378 | +0.18 | −0.05 | −0.850 | 2.636 |
| ATOM | 18 | O21 | URI | 1 | −0.456 | −6.019 | −2.230 | +0.16 | −0.15 | −0.510 | 2.636 |
| ATOM | 19 | P22 | URI | 1 | 0.309 | −5.557 | −3.556 | −0.22 | +0.49 | +1.210 | 2.636 |
| ATOM | 20 | O25 | URI | 1 | 1.198 | −6.590 | −4.145 | +0.07 | −0.37 | −0.850 | 2.636 |
| ATOM | 21 | O24 | URI | 1 | −0.816 | −4.998 | −4.348 | +0.05 | −0.20 | −0.850 | 2.636 |
| ATOM | 22 | O23 | URI | 1 | 1.321 | −4.399 | −3.063 | +0.14 | −0.24 | −0.368 | 2.636 |
| ATOM | 23 | C37 | URI | 1 | 0.889 | −3.298 | −3.831 | −0.16 | +0.32 | +0.227 | 2.636 |
| ATOM | 24 | C45 | URI | 1 | 2.099 | −2.553 | −4.476 | −0.43 | +0.25 | +0.211 | 2.636 |
| ATOM | 25 | N41 | URI | 1 | 2.929 | −3.526 | −5.238 | −0.23 | −0.64 | −0.650 | 2.636 |
| ATOM | 26 | H41 | URI | 1 | 3.603 | −4.049 | −4.679 | +0.09 | +0.46 | +0.440 | 2.636 |
| ATOM | 27 | C49 | URI | 1 | 2.759 | −3.779 | −6.540 | −0.49 | +0.37 | +0.396 | 2.636 |
| ATOM | 28 | O38 | URI | 1 | 1.946 | −3.192 | −7.255 | −0.06 | −0.30 | −0.396 | 2.636 |
| ATOM | 29 | C50 | URI | 1 | 3.650 | −4.873 | −7.122 | −0.56 | +0.00 | +0.000 | 2.636 |
| ATOM | 30 | C44 | URI | 1 | 2.908 | −1.789 | −3.382 | −0.42 | +0.08 | +0.113 | 2.636 |
| ATOM | 31 | O47 | URI | 1 | 3.896 | −0.960 | −3.988 | −0.13 | −0.45 | −0.537 | 2.636 |
| ATOM | 32 | H47 | URI | 1 | 3.658 | −0.028 | −3.798 | +0.01 | +0.25 | +0.424 | 2.636 |
| ATOM | 33 | C43 | URI | 1 | 2.007 | −0.898 | −2.490 | −0.44 | +0.06 | +0.113 | 2.636 |
| ATOM | 34 | O48 | URI | 1 | 2.794 | −0.359 | −1.434 | +0.08 | +0.00 | −0.537 | 2.636 |
| ATOM | 35 | H48 | URI | 1 | 3.587 | −0.930 | −1.401 | −0.19 | −0.23 | +0.424 | 2.636 |
| ATOM | 36 | C46 | URI | 1 | 0.815 | −1.713 | −1.918 | −0.29 | +0.07 | +0.113 | 2.636 |
| ATOM | 37 | C42 | URI | 1 | −0.202 | −0.853 | −1.150 | −0.33 | +0.02 | +0.113 | 2.636 |
| ATOM | 38 | O39 | URI | 1 | −0.162 | −1.183 | 0.234 | +0.14 | +0.06 | −0.537 | 2.636 |
| ATOM | 39 | H39 | URI | 1 | 0.762 | −1.053 | 0.548 | +0.11 | −0.03 | +0.424 | 2.636 |
| ATOM | 40 | O40 | URI | 1 | 0.110 | −2.381 | −3.007 | −0.26 | −0.42 | −0.227 | 2.636 |
| ATOM | 41 | C12 | URI | 1 | −3.906 | −8.224 | −2.904 | −0.23 | +0.06 | +0.113 | 2.636 |
| ATOM | 42 | O15 | URI | 1 | −4.204 | −7.378 | −4.019 | +0.01 | −0.39 | −0.537 | 2.636 |
| ATOM | 43 | H15 | URI | 1 | −4.590 | −6.571 | −3.632 | +0.08 | +0.18 | +0.424 | 2.636 |
| ATOM | 44 | C10 | URI | 1 | −3.901 | −9.678 | −3.428 | −0.28 | +0.08 | +0.113 | 2.636 |
| ATOM | 45 | O11 | URI | 1 | −4.853 | −9.822 | −4.456 | −0.04 | −0.63 | −0.537 | 2.636 |
| ATOM | 46 | H11 | URI | 1 | −5.076 | −10.776 | −4.450 | +0.07 | +0.49 | +0.424 | 2.636 |
| TER | | | | | | | | | | | |
| ENDMDL | | | | | | | | | | | |
| MODEL | 34 | | | | | | | | | | |
| USER | Run = 34 | | | | | | | | | | |
| USER | Cluster Rank = 37 | | | | | | | | | | |
| USER | Number of conformations in this cluster = 1 | | | | | | | | | | |
| USER | | | | | | | | | | | |
| USER | RMSD from reference structure = 3.409 A | | | | | | | | | | |
| USER | | | | | | | | | | | |
| USER | Estimated Free Energy of Binding = −4.27 kcal/mol [= (1) + (3)] | | | | | | | | | | |
| USER | Estimated Inhibition Constant, Ki = +7.44e−04 [Temperature = 298.15 K] | | | | | | | | | | |
| USER | | | | | | | | | | | |
| USER | Final Docked Energy = −7.32 kcal/mol [= (1) + (2)] | | | | | | | | | | |
| USER | | | | | | | | | | | |
| USER | (1) Final Intermolecular Energy = −8.94 kcal/mol | | | | | | | | | | |
| USER | (2) Final Internal Energy of Ligand = +1.62 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 −4.439000 | | | | | | | | | | |
| USER | NEWDPF tran0 1.816965 −4.775832 0.174741 | | | | | | | | | | |
| USER | NEWDPF quat0 0.294466 −0.657499 −0.693530 63.981762 | | | | | | | | | | |
| USER | NEWDPF ndihe 15 | | | | | | | | | | |
| USER | NEWDPF dihe0 −179.99 −115.98 107.34 164.56 162.03 −132.01 −62.34 29.66 | | | | | | | | | | |
| | 34.37 104.93 −38.83 −78.43 −146.10 −50.60 29.54 | | | | | | | | | | |
| USER | | | | | | | | | | | |
| USER | | | | | x | y | z | vdW | Elec | q | RMS |
| ATOM | 1 | C2 | URI | 1 | −2.328 | −8.213 | −6.292 | −0.46 | +0.20 | +0.296 | 3.409 |
| ATOM | 2 | N3 | URI | 1 | −3.377 | −9.089 | −6.405 | −0.33 | −0.27 | −0.292 | 3.409 |
| ATOM | 3 | C4 | URI | 1 | −4.369 | −9.317 | −5.449 | −0.48 | +0.39 | +0.346 | 3.409 |
| ATOM | 4 | C5 | URI | 1 | −4.226 | −8.552 | −4.251 | −0.36 | +0.00 | +0.000 | 3.409 |
| ATOM | 5 | C6 | URI | 1 | −3.206 | −7.689 | −4.133 | −0.30 | −0.03 | −0.050 | 3.409 |
| ATOM | 6 | N1 | URI | 1 | −2.242 | −7.515 | −5.120 | −0.21 | +0.02 | +0.039 | 3.409 |
| ATOM | 7 | O8 | URI | 1 | −5.242 | −10.146 | −5.705 | −0.07 | −0.50 | −0.396 | 3.409 |
| ATOM | 8 | O7 | URI | 1 | −1.500 | −8.074 | −7.194 | −0.03 | −0.18 | −0.396 | 3.409 |
| ATOM | 9 | H3 | URI | 1 | −3.437 | −9.617 | −7.257 | +0.07 | +0.30 | +0.393 | 3.409 |
| ATOM | 10 | C9 | URI | 1 | −1.134 | −6.571 | −4.927 | −0.29 | +0.03 | +0.174 | 3.409 |
| ATOM | 11 | O14 | URI | 1 | −0.072 | −7.316 | −4.343 | +0.08 | −0.08 | −0.227 | 3.409 |
| ATOM | 12 | C13 | URI | 1 | 0.827 | −6.406 | −3.653 | −0.29 | +0.05 | +0.113 | 3.409 |
| ATOM | 13 | C16 | URI | 1 | 1.208 | −7.026 | −2.308 | −0.34 | +0.04 | +0.113 | 3.409 |
| ATOM | 14 | O17 | URI | 1 | 2.620 | −7.031 | −2.217 | +0.11 | −0.17 | −0.368 | 3.409 |
| ATOM | 15 | P18 | URI | 1 | 3.704 | −5.887 | −2.129 | −0.61 | +1.16 | +1.210 | 3.409 |
| ATOM | 16 | O19 | URI | 1 | 4.960 | −6.491 | −1.601 | −0.55 | −1.51 | −0.850 | 3.409 |
| ATOM | 17 | O20 | URI | 1 | 3.757 | −5.180 | −3.448 | −0.05 | −0.86 | −0.850 | 3.409 |
| ATOM | 18 | O21 | URI | 1 | 2.971 | −5.067 | −0.974 | +0.04 | −0.32 | −0.510 | 3.409 |
| ATOM | 19 | P22 | URI | 1 | 3.355 | −4.732 | 0.541 | −0.37 | +0.64 | +1.210 | 3.409 |
| ATOM | 20 | O25 | URI | 1 | 3.098 | −5.836 | 1.500 | +0.06 | −0.36 | −0.850 | 3.409 |
| ATOM | 21 | O24 | URI | 1 | 4.729 | −4.194 | 0.371 | +0.16 | −0.85 | −0.850 | 3.409 |
| ATOM | 22 | O23 | URI | 1 | 2.316 | −3.577 | 0.982 | +0.16 | −0.09 | −0.368 | 3.409 |
| ATOM | 23 | C37 | URI | 1 | 2.874 | −2.441 | 0.361 | −0.16 | +0.04 | +0.227 | 3.409 |
| ATOM | 24 | C45 | URI | 1 | 2.976 | −1.247 | 1.360 | −0.19 | +0.00 | +0.211 | 3.409 |
| ATOM | 25 | N41 | URI | 1 | 3.711 | −1.684 | 2.579 | −0.10 | −0.07 | −0.650 | 3.409 |
| ATOM | 26 | H41 | URI | 1 | 3.132 | −2.111 | 3.301 | +0.11 | +0.03 | +0.440 | 3.409 |
| ATOM | 27 | C49 | URI | 1 | 5.041 | −1.617 | 2.701 | −0.31 | +0.08 | +0.396 | 3.409 |
| ATOM | 28 | O38 | URI | 1 | 5.787 | −1.130 | 1.850 | −0.05 | −0.08 | −0.396 | 3.409 |
| ATOM | 29 | C50 | URI | 1 | 5.615 | −2.195 | 3.990 | −0.20 | +0.00 | +0.000 | 3.409 |
| ATOM | 30 | C44 | URI | 1 | 1.555 | −0.683 | 1.676 | −0.14 | −0.02 | +0.113 | 3.409 |
| ATOM | 31 | O47 | URI | 1 | 1.659 | 0.518 | 2.435 | +0.17 | +0.17 | −0.537 | 3.409 |
| ATOM | 32 | H47 | URI | 1 | 0.784 | 0.962 | 2.409 | +0.10 | −0.25 | +0.424 | 3.409 |
| ATOM | 33 | C43 | URI | 1 | 0.738 | −0.379 | 0.395 | −0.21 | −0.01 | +0.113 | 3.409 |
| ATOM | 34 | O48 | URI | 1 | −0.586 | −0.016 | 0.769 | +0.09 | +0.22 | −0.537 | 3.409 |
| ATOM | 35 | H48 | URI | 1 | −1.003 | −0.855 | 1.050 | +0.10 | −0.19 | +0.424 | 3.409 |
| ATOM | 36 | C46 | URI | 1 | 0.716 | −1.610 | −0.553 | −0.20 | +0.02 | +0.113 | 3.409 |
| ATOM | 37 | C42 | URI | 1 | 0.052 | −1.326 | −1.910 | −0.34 | +0.07 | +0.113 | 3.409 |
| ATOM | 38 | O39 | URI | 1 | −1.359 | −1.245 | −1.746 | −0.04 | −0.09 | −0.537 | 3.409 |
| ATOM | 39 | H39 | URI | 1 | −1.697 | −2.157 | −1.589 | −0.05 | −0.01 | +0.424 | 3.409 |
| ATOM | 40 | O40 | URI | 1 | 2.081 | −2.061 | −0.804 | +0.12 | −0.05 | −0.227 | 3.409 |
| ATOM | 41 | C12 | URI | 1 | 0.052 | −5.097 | −3.510 | −0.23 | +0.05 | +0.113 | 3.409 |
| ATOM | 42 | O15 | URI | 1 | 0.499 | −4.161 | −4.496 | −0.12 | −0.45 | −0.537 | 3.409 |
| ATOM | 43 | H15 | URI | 1 | 0.003 | −4.387 | −5.305 | −0.19 | +0.05 | +0.424 | 3.409 |
| ATOM | 44 | C10 | URI | 1 | −1.398 | −5.485 | −3.881 | −0.28 | +0.03 | +0.113 | 3.409 |
| ATOM | 45 | O11 | URI | 1 | −2.056 | −4.385 | −4.465 | +0.04 | −0.02 | −0.537 | 3.409 |
| ATOM | 46 | H11 | URI | 1 | −2.984 | −4.682 | −4.567 | +0.05 | +0.02 | +0.424 | 3.409 |
| TER | | | | | | | | | | | |
| ENDMDL | | | | | | | | | | | |
| MODEL | 43 | | | | | | | | | | |
| USER | Run = 43 | | | | | | | | | | |
| USER | Cluster Rank = 38 | | | | | | | | | | |
| USER | Number of conformations in this cluster = 1 | | | | | | | | | | |
| USER | | | | | | | | | | | |
| USER | RMSD from reference structure = 6.135 A | | | | | | | | | | |
| USER | | | | | | | | | | | |
| USER | Estimated Free Energy of Binding = −3.55 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −7.09 kcal/mol [= (1) + (2)] | | | | | | | | | | |
| USER | | | | | | | | | | | |
| USER | (1) Final Intermolecular Energy = −8.22 kcal/mol | | | | | | | | | | |
| USER | (2) Final Internal Energy of Ligand = +1.12 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = doublecheck.dpf | | | | | | | | | | |
| USER | NEWDPF move doublecheck.pdbq | | | | | | | | | | |
| USER | NEWDPF about 1.052000 −3.904000 4.439000 | | | | | | | | | | |
| USER | NEWDPF tran0 −2.862016 −2.604417 0.788429 | | | | | | | | | | |
| USER | NEWDPF quat0 0.556389 0.791880 −0.251709 54.591137 | | | | | | | | | | |
| USER | NEWDPF ndihe 15 | | | | | | | | | | |
| USER | NEWDPF dihe0 134.88 17.23 75.18 22.66 43.89 −149.12 −36.06 14.38 −24.34 | | | | | | | | | | |
| | 13.27 131.06 121.36 −172.11 −124.55 13.83 | | | | | | | | | | |
| USER | | | | | | | | | | | |
| USER | | | | | x | y | z | vdW | Elec | q | RMS |
| ATOM | 1 | C2 | URI | 1 | −8.713 | −8.289 | −1.277 | −0.44 | +0.06 | +0.296 | 6.135 |
| ATOM | 2 | N3 | URI | 1 | −8.734 | −9.658 | −1.368 | −0.23 | −0.14 | −0.292 | 6.135 |
| ATOM | 3 | C4 | URI | 1 | −7.892 | −10.543 | −0.692 | −0.21 | +0.14 | +0.346 | 6.135 |
| ATOM | 4 | C5 | URI | 1 | −6.924 | −9.916 | 0.152 | −0.12 | +0.00 | +0.000 | 6.135 |
| ATOM | 5 | C6 | URI | 1 | −6.899 | −8.578 | 0.250 | −0.17 | −0.01 | −0.050 | 6.135 |
| ATOM | 6 | N1 | URI | 1 | −7.760 | −7.749 | −0.459 | −0.23 | +0.00 | +0.039 | 6.135 |
| ATOM | 7 | O8 | URI | 1 | −8.038 | −11.749 | −0.891 | +0.14 | −0.19 | −0.396 | 6.135 |
| ATOM | 8 | O7 | URI | 1 | −9.501 | −7.586 | −1.910 | −0.05 | −0.04 | −0.396 | 6.135 |
| ATOM | 9 | H3 | URI | 1 | −9.421 | −10.061 | −1.979 | +0.07 | +0.28 | +0.393 | 6.135 |
| ATOM | 10 | C9 | URI | 1 | −7.672 | −6.289 | −0.328 | −0.50 | −0.06 | +0.174 | 6.135 |
| ATOM | 11 | O14 | URI | 1 | −6.528 | −5.889 | −1.072 | −0.01 | +0.04 | −0.227 | 6.135 |
| ATOM | 12 | C13 | URI | 1 | −6.068 | −4.599 | −0.586 | −0.60 | −0.05 | +0.113 | 6.135 |
| ATOM | 13 | C16 | URI | 1 | −4.543 | −4.634 | −0.480 | −0.36 | −0.03 | +0.113 | 6.135 |
| ATOM | 14 | O17 | URI | 1 | −4.030 | −5.047 | −1.733 | +0.06 | +0.03 | −0.368 | 6.135 |
| ATOM | 15 | P18 | URI | 1 | −4.427 | −4.723 | −3.225 | −0.58 | −0.02 | +1.210 | 6.135 |
| ATOM | 16 | O19 | URI | 1 | −5.569 | −3.766 | −3.196 | −0.26 | +0.19 | −0.850 | 6.135 |
| ATOM | 17 | O20 | URI | 1 | −3.189 | −4.327 | −3.970 | −0.11 | −0.06 | −0.850 | 6.135 |
| ATOM | 18 | O21 | URI | 1 | −4.957 | −6.187 | −3.567 | −0.03 | −0.15 | −0.510 | 6.135 |
| ATOM | 19 | P22 | URI | 1 | −4.221 | −7.574 | −3.868 | −0.20 | +0.86 | +1.210 | 6.135 |
| ATOM | 20 | O25 | URI | 1 | −5.119 | −8.658 | −4.339 | −0.02 | −1.27 | −0.850 | 6.135 |
| ATOM | 21 | O24 | URI | 1 | −3.093 | −7.115 | −4.717 | −0.09 | −0.63 | −0.850 | 6.135 |
| ATOM | 22 | O23 | URI | 1 | −3.707 | −8.087 | −2.425 | +0.16 | −0.14 | −0.368 | 6.135 |
| ATOM | 23 | C37 | URI | 1 | −2.401 | −8.523 | −2.728 | −0.20 | +0.09 | +0.227 | 6.135 |
| ATOM | 24 | C45 | URI | 1 | −1.995 | −9.741 | −1.841 | −0.26 | +0.08 | +0.211 | 6.135 |
| ATOM | 25 | N41 | URI | 1 | −3.026 | −10.808 | −1.966 | −0.11 | −0.30 | −0.650 | 6.135 |
| ATOM | 26 | H41 | URI | 1 | −3.963 | −10.539 | −1.666 | +0.11 | +0.19 | +0.440 | 6.135 |
| ATOM | 27 | C49 | URI | 1 | −2.786 | −12.004 | −2.513 | −0.32 | +0.22 | +0.396 | 6.135 |
| ATOM | 28 | O38 | URI | 1 | −1.678 | −12.370 | −2.910 | −0.05 | −0.24 | −0.396 | 6.135 |
| ATOM | 29 | C50 | URI | 1 | −3.992 | −12.930 | −2.631 | −0.25 | +0.00 | +0.000 | 6.135 |
| ATOM | 30 | C44 | URI | 1 | −1.768 | −9.285 | −0.366 | −0.22 | +0.03 | +0.113 | 6.135 |
| ATOM | 31 | O47 | URI | 1 | −1.218 | −10.352 | 0.402 | +0.09 | −0.15 | −0.537 | 6.135 |
| ATOM | 32 | H47 | URI | 1 | −0.352 | −10.051 | 0.750 | +0.09 | +0.07 | +0.424 | 6.135 |
| ATOM | 33 | C43 | URI | 1 | −0.813 | −8.068 | −0.260 | −0.23 | +0.02 | +0.113 | 6.135 |
| ATOM | 34 | O48 | URI | 1 | −0.780 | −7.626 | 1.091 | +0.15 | −0.07 | −0.537 | 6.135 |
| ATOM | 35 | H48 | URI | 1 | −0.453 | −8.397 | 1.596 | +0.10 | +0.06 | +0.424 | 6.135 |
| ATOM | 36 | C46 | URI | 1 | −1.276 | −6.918 | −1.198 | −0.14 | +0.02 | +0.113 | 6.135 |
| ATOM | 37 | C42 | URI | 1 | −0.288 | −5.743 | −1.257 | −0.14 | +0.03 | +0.113 | 6.135 |
| ATOM | 38 | O39 | URI | 1 | −0.630 | −4.778 | −0.268 | +0.19 | −0.06 | −0.537 | 6.135 |
| ATOM | 39 | H39 | URI | 1 | −1.055 | −4.016 | −0.724 | +0.11 | +0.04 | +0.424 | 6.135 |
| ATOM | 40 | O40 | URI | 1 | −1.450 | −7.430 | −2.553 | +0.15 | −0.07 | −0.227 | 6.135 |
| ATOM | 41 | C12 | URI | 1 | −6.758 | −4.402 | 0.763 | −0.70 | −0.07 | +0.113 | 6.135 |
| ATOM | 42 | O15 | URI | 1 | −7.865 | −3.507 | 0.612 | −0.20 | +0.48 | −0.537 | 6.135 |
| ATOM | 43 | H15 | URI | 1 | −7.481 | −2.682 | 0.262 | −0.25 | −0.58 | +0.424 | 6.135 |
| ATOM | 44 | C10 | URI | 1 | −7.344 | −5.796 | 1.083 | −0.54 | −0.04 | +0.113 | 6.135 |
| ATOM | 45 | O11 | URI | 1 | −8.521 | −5.659 | 1.846 | −0.06 | +0.38 | −0.537 | 6.135 |
| ATOM | 46 | H11 | URI | 1 | −9.167 | −5.258 | 1.228 | −0.30 | −0.42 | +0.424 | 6.135 |

| | |
|---|---|
| TER | |
| ENDMDL | |
| MODEL | 25 |
| USER | Run = 25 |
| USER | Cluster Rank = 39 |
| USER | Number of conformations in this cluster = 1 |
| USER | |
| USER | RMSD from reference structure = 4.180 A |
| USER | |
| USER | Estimated Free Energy of Binding = −5.49 kcal/mol [= (1) + (3)] |
| USER | Estimated Inhibition Constant, Ki = +9.41e−05 [Temperature = 298.15 K] |
| USER | |
| USER | Final Docked Energy = −6.96 kcal/mol [= (1) + (2)] |
| USER | |
| USER | (1) Final Intermolecular Energy = −10.16 kcal/mol |
| USER | (2) Final Internal Energy of Ligand = +3.20 kcal/mol |
| USER | (3) Torsional Free Energy = +4.67 kcal/mol |
| USER | |
| USER | |
| USER | DPF = doublecheck.dpf |
| USER | NEWDPF move doublecheck.pdbq |
| USER | NEWDPF about 1.052000 −3.904000 −4.439000 |
| USER | NEWDPF tran0 −8.485990 −14.554324 −6.446734 |
| USER | NEWDPF quat0 0.301213 −0.919115 0.253964 165.205355 |
| USER | NEWDPF ndihe 15 |
| USER | NEWDPF dihe0 178.03 −179.51 125.06 157.87 28.20 142.11 −1.08 29.36 |
| | −45.80 49.43 22.00 24.29 178.09 −65.54 −169.25 |
| USER | |

TABLE 19
LOWEST ENERGY DOCKED CONFORMATION from EACH CLUSTER
Residue number will be set to the conformation's cluster rank.

| MODEL | 1 | | | | | | | | | | |
|---|---|---|---|---|---|---|---|---|---|---|---|
| USER | Run = 1 | | | | | | | | | | |
| USER | Cluster Rank = 1 | | | | | | | | | | |
| USER | Number of conformations in this cluster = 1 | | | | | | | | | | |
| USER | | | | | | | | | | | |
| USER | RMSD from reference structure = 3.720 A | | | | | | | | | | |
| USER | | | | | | | | | | | |
| USER | Estimated Free Energy of Binding = −6.83 kcal/mol [= (l) + (3)] | | | | | | | | | | |
| USER | Estimated Inhibition Constant, Ki = +9.91e−06 [Temperature = 298.15 K] | | | | | | | | | | |
| USER | | | | | | | | | | | |
| USER | Final Docked Energy = −10.82 kcal/mol [= (l) + (2)] | | | | | | | | | | |
| USER | | | | | | | | | | | |
| USER | (1) Final Intermolecular Energy = −11.50 kcal/mol | | | | | | | | | | |
| USER | (2) Final Internal Energy of Ligand = +0.67 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWUFF move udpg_ts.pdbq | | | | | | | | | | |
| USER | NEWDPF about −1.182000 20.257999 9.670000 | | | | | | | | | | |
| USER | NEWDPF tran0 6.332435 20.507966 18.600877 | | | | | | | | | | |
| USER | NEWDPF quat0 −0.573187 −0.516558 −0.636101 99.290823 − | | | | | | | | | | |
| USER | NEWDPF ndihe 15 | | | | | | | | | | |
| USER | NEWDPF dihe0 −88.28 −179.98 41.74 177.53 18.76 −13.82 −170.61 83.42 | | | | | | | | | | |
| 104.83 | 13.29 −101.72 97.93 107.26 −178.59 158.24 | | | | | | | | | | |
| USER | | | | | | | | | | | |
| USER | | | | Rank | x | y | z | vdW | Elec | q | RMS |
| ATOM | 1 | N1 | MOL | 1 | 6.283 | 17.847 | 19.033 | −0.20 | −0.26 | −0.211 | 3.720 |
| ATOM | 2 | C8 | MOL | 1 | 7.564 | 17.587 | 19.504 | −0.47 | +0.00 | +0.000 | 3.720 |
| ATOM | 3 | C6 | MOL | 1 | 8.622 | 17.422 | 18.695 | −0.68 | +0.00 | +0.000 | 3.720 |
| ATOM | 4 | CS | MOL | 1 | 8.441 | 17.481 | 17.278 | −0.37 | +0.57 | +0.396 | 3.720 |
| ATOM | 5 | O11 | MOL | 1 | 9.306 | 17.355 | 16.411 | −0.07 | −0.57 | −0.396 | 3.720 |
| ATOM | 6 | N3 | MOL | 1 | 7.131 | 17.734 | 16.870 | +0.64 | −0.60 | −0.440 | 3.720 |
| ATOM | 7 | H3 | MOL | 1 | 6.965 | 17.796 | 15.855 | −0.07 | +0.53 | +0.440 | 3.720 |
| ATOM | 8 | C2 | MOL | 1 | 6.045 | 17.909 | 17.688 | −0.41 | +0.47 | +0.396 | 3.720 |
| ATOM | 9 | O10 | MOL | 1 | 4.919 | 18.125 | 17.238 | +0.03 | −0.41 | −0.396 | 3.720 |
| ATOM | 10 | C12 | MOL | 1 | 5.166 | 18.043 | 19.968 | −0.31 | +0.40 | +0.324 | 3.720 |
| ATOM | 11 | O22 | MOL | 1 | 4.057 | 17.338 | 19.420 | +0.10 | −0.26 | −0.227 | 3.720 |
| ATOM | 12 | C20 | MOL | 1 | 2.826 | 17.888 | 19.961 | −0.52 | +0.18 | +0.113 | 3.720 |
| ATOM | 13 | C25 | MOL | 1 | 1.811 | 18.024 | 18.824 | −0.37 | +0.21 | +0.113 | 3.720 |
| ATOM | 14 | O28 | MOL | 1 | 2.392 | 18.831 | 17.818 | +0.04 | −0.56 | −0.368 | 3.720 |
| ATOM | 15 | P29 | MOL | 1 | 2.287 | 20.365 | 17.466 | −0.39 | +1.59 | +1.019 | 3.720 |
| ATOM | 16 | O30 | MOL | 1 | 1.017 | 20.880 | 18.053 | −0.03 | −0.62 | −0.255 | 3.720 |
| ATOM | 17 | O31 | MOL | 1 | 3.569 | 21.033 | 17.851 | +0.05 | −0.33 | −0.255 | 3.720 |
| ATOM | 18 | O32 | MOL | 1 | 2.106 | 20.151 | 15.895 | +0.06 | −0.70 | −0.510 | 3.720 |
| ATOM | 19 | P33 | MOL | 1 | 3.108 | 20.263 | 14.653 | −0.52 | +1.45 | +1.019 | 3.720 |
| ATOM | 20 | O35 | MOL | 1 | 4.188 | 19.336 | 15.080 | −0.06 | −0.32 | −0.255 | 3.720 |
| ATOM | 21 | O36 | MOL | 1 | 3.431 | 21.653 | 14.246 | −0.10 | −0.30 | −0.255 | 3.720 |
| ATOM | 22 | O34 | MOL | 1 | 2.275 | 19.666 | 13.399 | −0.35 | −0.84 | −0.368 | 3.720 |
| ATOM | 23 | C37 | MOL | 1 | 0.284 | 21.368 | 12.746 | −0.38 | +0.27 | +0.227 | 3.720 |
| ATOM | 24 | O39 | MOL | 1 | −0.153 | 20.475 | 11.983 | −0.03 | −0.29 | −0.227 | 3.720 |
| ATOM | 25 | C40 | MOL | 1 | −0.765 | 19.251 | 12.447 | −0.61 | +0.14 | +0.113 | 3.720 |
| ATOM | 26 | C50 | MOL | 1 | −1.436 | 18.681 | 11.211 | −0.70 | +0.13 | +0.113 | 3.720 |
| ATOM | 27 | O61 | MOL | 1 | −2.152 | 19.654 | 10.536 | −0.12 | −1.02 | −0.537 | 3.720 |
| ATOM | 28 | H61 | MOL | 1 | −1.529 | 20.131 | 9.967 | +0.06 | +0.69 | +0.424 | 3.720 |
| ATOM | 29 | C41 | MOL | 1 | −1.717 | 19.603 | 13.577 | −0.71 | +0.12 | +0.113 | 3.720 |
| ATOM | 30 | O48 | MOL | 1 | −2.251 | 18.444 | 14.170 | −0.14 | −0.37 | −0.537 | 3.720 |
| ATOM | 31 | H48 | MOL | 1 | −1.549 | 17.922 | 14.531 | −0.23 | +0.18 | +0.424 | 3.720 |
| ATOM | 32 | C42 | MOL | 1 | −0.959 | 20.348 | 14.663 | −0.60 | +0.13 | +0.113 | 3.720 |
| ATOM | 33 | O46 | MOL | 1 | −1.804 | 20.710 | 15.712 | −0.10 | −0.67 | −0.537 | 3.720 |
| ATOM | 34 | H46 | MOL | 1 | −1.259 | 20.877 | 16.471 | +0.06 | +0.66 | +0.424 | 3.720 |
| ATOM | 35 | C43 | MOL | 1 | −0.358 | 21.629 | 14.086 | −0.43 | +0.24 | +0.211 | 3.720 |
| ATOM | 36 | N51 | MOL | 1 | 0.540 | 22.274 | 14.999 | −0.28 | −0.76 | −0.650 | 3.720 |
| ATOM | 37 | H51 | MOL | 1 | 1.473 | 21.864 | 15.141 | +0.10 | +0.52 | +0.440 | 3.720 |
| ATOM | 38 | C53 | MOL | 1 | 0.216 | 23.400 | 15.683 | −0.70 | +0.52 | +0.396 | 3.720 |
| ATOM | 39 | O54 | MOL | 1 | −0.781 | 24.034 | 15.478 | −0.39 | −0.58 | −0.396 | 3.720 |
| ATOM | 40 | C55 | MOL | 1 | 1.206 | 23.783 | 16.760 | −0.66 | +0.00 | +0.000 | 3.720 |
| ATOM | 41 | C18 | MOL | 1 | 3.216 | 19.232 | 20.577 | −0.56 | +0.21 | +0.113 | 3.720 |
| ATOM | 42 | O23 | MOL | 1 | 3.317 | 19.099 | 21.998 | −0.02 | −0.97 | −0.537 | 3.720 |
| ATOM | 43 | H23 | MOL | 1 | 3.944 | 18.313 | 22.225 | +0.07 | +0.62 | +0.424 | 3.720 |
| ATOM | 44 | C14 | MOL | 1 | 4.646 | 19.480 | 20.043 | −0.37 | +0.16 | +0.113 | 3.720 |
| ATOM | 45 | O16 | MOL | 1 | 5.385 | 20.224 | 20.983 | −0.02 | −0.91 | −0.537 | 3.720 |
| ATOM | 46 | H16 | MOL | 1 | 5.511 | 21.186 | 20.637 | +0.03 | +0.61 | +0.424 | 3.720 |
| TER | | | | | | | | | | | |
| ENDMDL | | | | | | | | | | | |
| MODEL | 6 | | | | | | | | | | |
| USER | Run = 6 | | | | | | | | | | |
| USER | Cluster Rank = 2 | | | | | | | | | | |
| USER | Number of conformations in this cluster = 1 | | | | | | | | | | |
| USER | | | | | | | | | | | |
| USER | RMSD from reference structure = 4.547 A | | | | | | | | | | |
| USER | | | | | | | | | | | |
| USER | Estimated Free Energy of Binding = −8.00 kcal/MOL [= (1) + (3)] | | | | | | | | | | |
| USER | Estimated Inhibition Constant, Ki = +1.36e−06[Temperature = 298.15 K] | | | | | | | | | | |
| USER | | | | | | | | | | | |
| USER | Final Docked Energy = −9.99 kcal/mol [= (1) + (2)] | | | | | | | | | | |
| USER | | | | | | | | | | | |
| USER | (1) Final Intermolecular Energy = −12.67 kcal/mol | | | | | | | | | | |
| USER | (2) Final Internal Energy of Ligand = +2.68 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 8.207197 17.387842 21.188238 | | | | | | | | | | |
| USER | NEWDPF quat0 0.682501 0.655153 0.323987 143.529647 | | | | | | | | | | |
| USER | NEWDPF ndihe 15 | | | | | | | | | | |
| USER | NEWDPF dihe0 82.68 −2.46 −95.91 −179.97 −64.21 179.57 180.00 149.33 −112.22 | | | | | | | | | | |
| | −149.66 −85.97 −179.45 −24.07 −32.21 148.52 | | | | | | | | | | |
| USER | | | | | | | | | | | |
| USER | | | | Rank | x | y | z | vdW | Elec | q | RMS |
| ATOM | 1 | N1 | MOL | 2 | 6.049 | 18.605 | 22.252 | −0.26 | −0.32 | −0.211 | 4.547 |
| ATOM | 2 | C8 | MOL | 2 | 6.598 | 19.371 | 23.273 | −0.50 | +0.00 | +0.000 | 4.547 |
| ATOM | 3 | C6 | MOL | 2 | 6.897 | 18.873 | 24.482 | −0.71 | +0.00 | +0.000 | 4.547 |
| ATOM | 4 | C5 | MOL | 2 | 6.616 | 17.501 | 24.768 | −0.74 | +0.45 | +0.396 | 4.547 |
| ATOM | 5 | O11 | MOL | 2 | 6.834 | 16.899 | 25.820 | −0.01 | −0.38 | −0.396 | 4.547 |
| ATOM | 6 | N3 | MOL | 2 | 6.056 | 16.789 | 23.707 | −0.42 | −0.45 | −0.440 | 4.547 |
| ATOM | 7 | H3 | MOL | 2 | 5.851 | 15.794 | 23.872 | +0.05 | +0.39 | +0.440 | 4.547 |
| ATOM | 8 | C2 | MOL | 2 | 5.752 | 17.288 | 22.466 | −0.53 | +0.45 | +0.396 | 4.547 |
| ATOM | 9 | O10 | MOL | 2 | 5.253 | 16.584 | 21.587 | −0.01 | −0.40 | −0.396 | 4.547 |
| ATOM | 10 | C12 | MOL | 2 | 5.765 | 19.199 | 20.938 | −0.38 | +0.49 | +0.324 | 4.547 |
| ATOM | 11 | O22 | MOL | 2 | 4.618 | 18.524 | 20.433 | +0.09 | −0.31 | −0.227 | 4.547 |
| ATOM | 12 | C20 | MOL | 2 | 4.583 | 18.656 | 18.986 | −0.30 | +0.14 | +0.113 | 4.547 |
| ATOM | 13 | C25 | MOL | 2 | 4.205 | 17.302 | 18.383 | −0.35 | +0.12 | +0.113 | 4.547 |
| ATOM | 14 | O28 | MOL | 2 | 2.795 | 17.252 | 18.282 | +0.03 | −0.44 | −0.368 | 4.547 |
| ATOM | 15 | P29 | MOL | 2 | 1.806 | 16.854 | 17.119 | −0.67 | +1.02 | +1.019 | 4.547 |
| ATOM | 16 | O30 | MOL | 2 | 2.079 | 15.435 | 16.756 | −0.23 | −0.16 | −0.255 | 4.547 |
| ATOM | 17 | O31 | MOL | 2 | 0.415 | 17.229 | 17.526 | −0.05 | −0.39 | −0.255 | 4.547 |
| ATOM | 18 | O32 | MOL | 2 | 2.453 | 17.823 | 16.028 | +0.09 | −0.50 | −0.510 | 4.547 |
| ATOM | 19 | P33 | MOL | 2 | 2.242 | 19.369 | 15.676 | −0.37 | +1.45 | +1.019 | 4.547 |
| ATOM | 20 | O35 | MOL | 2 | 1.506 | 19.266 | 14.391 | −0.33 | −0.57 | −0.255 | 4.547 |
| ATOM | 21 | O36 | MOL | 2 | 3.472 | 20.195 | 15.773 | +0.02 | −0.33 | −0.255 | 4.547 |
| ATOM | 22 | O34 | MOL | 2 | 1.293 | 19.933 | 16.861 | −0.14 | −0.63 | −0.368 | 4.547 |
| ATOM | 23 | C37 | MOL | 2 | −0.315 | 21.870 | 15.884 | −0.61 | +0.30 | +0.227 | 4.547 |
| ATOM | 24 | O39 | MOL | 2 | 0.426 | 22.699 | 16.463 | −0.06 | −0.32 | −0.227 | 4.547 |
| ATOM | 25 | C40 | MOL | 2 | 1.775 | 23.002 | 16.041 | −0.51 | +0.13 | +0.113 | 4.547 |
| ATOM | 26 | C50 | MOL | 2 | 2.121 | 24.274 | 16.793 | −0.67 | +0.14 | +0.113 | 4.547 |
| ATOM | 27 | O61 | MOL | 2 | 3.466 | 24.315 | 17.116 | −0.07 | −0.53 | −0.537 | 4.547 |
| ATOM | 28 | H61 | MOL | 2 | 3.543 | 24.134 | 18.065 | −0.01 | +0.36 | +0.424 | 4.547 |
| ATOM | 29 | C41 | MOL | 2 | 1.771 | 23.143 | 14.529 | −0.60 | +0.11 | +0.113 | 4.547 |
| ATOM | 30 | O48 | MOL | 2 | 3.077 | 23.307 | 14.032 | −0.02 | −0.37 | −0.537 | 4.547 |
| ATOM | 31 | H48 | MOL | 2 | 3.134 | 24.121 | 13.554 | −0.23 | +0.09 | +0.424 | 4.547 |
| ATOM | 32 | C42 | MOL | 2 | 1.214 | 21.875 | 13.904 | −0.52 | +0.13 | +0.113 | 4.547 |
| ATOM | 33 | O46 | MOL | 2 | 1.172 | 21.972 | 12.512 | −0.06 | −0.61 | −0.537 | 4.547 |
| ATOM | 34 | H46 | MOL | 2 | 1.802 | 22.634 | 12.255 | +0.03 | +0.42 | +0.424 | 4.547 |
| ATOM | 35 | C43 | MOL | 2 | −0.214 | 21.641 | 14.396 | −0.51 | +0.24 | +0.211 | 4.547 |
| ATOM | 36 | N51 | MOL | 2 | −0.725 | 20.359 | 14.011 | −0.34 | −0.76 | −0.650 | 4.547 |
| ATOM | 37 | H51 | MOL | 2 | −0.281 | 19.516 | 14.402 | +0.09 | +0.56 | +0.440 | 4.547 |
| ATOM | 38 | C53 | MOL | 2 | −1.766 | 20.205 | 13.155 | −0.72 | +0.44 | +0.396 | 4.547 |
| ATOM | 39 | O54 | MOL | 2 | −2.441 | 21.115 | 12.764 | +0.04 | −0.47 | −0.396 | 4.547 |
| ATOM | 40 | C55 | MOL | 2 | −1.995 | 18.780 | 12.701 | −0.71 | +0.00 | +0.000 | 4.547 |
| ATOM | 41 | C18 | MOL | 2 | 5.985 | 19.118 | 18.585 | −0.36 | +0.16 | +0.113 | 4.547 |
| ATOM | 42 | O23 | MOL | 2 | 5.969 | 20.524 | 18.319 | −0.09 | −0.87 | −0.537 | 4.547 |
| ATOM | 43 | H23 | MOL | 2 | 5.595 | 21.024 | 19.139 | +0.06 | +0.56 | +0.424 | 4.547 |
| ATOM | 44 | C14 | MOL | 2 | 6.821 | 18.910 | 19.869 | −0.41 | +0.17 | +0.113 | 4.547 |
| ATOM | 45 | O16 | MOL | 2 | 7.855 | 19.864 | 19.927 | −0.11 | −1.02 | −0.537 | 4.547 |
| ATOM | 46 | H16 | MOL | 2 | 8.299 | 19.835 | 20.856 | +0.06 | +0.86 | +0.424 | 4.547 |
| TER | | | | | | | | | | | |
| ENDMDL | | | | | | | | | | | |
| MODEL | 10 | | | | | | | | | | |
| USER | Run = 10 | | | | | | | | | | |
| USER | Cluster Rank = 3 | | | | | | | | | | |
| USER | Number of conformations in this cluster = 1 | | | | | | | | | | |
| USER | | | | | | | | | | | |
| USER | RMSD from reference structure = 4.894 A | | | | | | | | | | |
| USER | | | | | | | | | | | |
| USER | Estimated Free Energy of Binding = −6.73 kcal/mol [= (1) + (3)] | | | | | | | | | | |
| USER | Estimated Inhibition Constant, Ki = +1.16e−05 [Temperature = 298.15 K] | | | | | | | | | | |
| USER | | | | | | | | | | | |
| USER | Final Docked Energy = −9.97 kcal/mol [= (1) + (2)] | | | | | | | | | | |
| USER | | | | | | | | | | | |
| USER | (1) Final Intermolecular Energy = −11.40 kcal/mol | | | | | | | | | | |
| USER | (2) Final Internal Energy of Ligand = +1.43 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 3.329947 22.332643 18.584840 | | | | | | | | | | |
| USER | NEWDPF quat0 0.564695 0.327976 −0.757332 116.616515 | | | | | | | | | | |
| USER | NEWDPF ndihe 15 | | | | | | | | | | |
| USER | NEWDPF dihe0 −172.37 −173.55 −128.85 −177.28 80.75 −175.42 −179.96 64.33 | | | | | | | | | | |
| | 146.62 −150.11 160.48 114.44 23.67 −126.19 33.99 | | | | | | | | | | |
| USER | | | | | | | | | | | |
| USER | | | | Rank | x | y | z | vdW | Elec | q | RMS |
| ATOM | 1 | N1 | MOL | 3 | 3.628 | 24.076 | 16.550 | −0.33 | −0.21 | −0.211 | 4.894 |
| ATOM | 2 | C8 | MOL | 3 | 4.767 | 23.607 | 15.906 | −0.39 | +0.00 | +0.000 | 4.894 |
| ATOM | 3 | C6 | MOL | 3 | 6.011 | 23.894 | 16.317 | −0.46 | +0.00 | +0.000 | 4.894 |
| ATOM | 4 | C5 | MOL | 3 | 6.201 | 24.747 | 17.449 | −0.40 | +0.42 | +0.396 | 4.894 |
| ATOM | 5 | O11 | MOL | 3 | 7.270 | 25.097 | 17.947 | +0.09 | −0.42 | −0.396 | 4.894 |
| ATOM | 6 | N3 | MOL | 3 | 5.020 | 25.193 | 18.043 | −0.27 | −0.35 | −0.440 | 4.894 |
| ATOM | 7 | H3 | MOL | 3 | 5.117 | 25.800 | 18.870 | +0.08 | +0.32 | +0.440 | 4.894 |
| ATOM | 8 | C2 | MOL | 3 | 3.744 | 24.901 | 17.634 | −0.56 | +0.35 | +0.396 | 4.894 |
| ATOM | 9 | O10 | MOL | 3 | 2.756 | 25.345 | 18.221 | −0.18 | −0.41 | −0.396 | 4.894 |
| ATOM | 10 | C12 | MOL | 3 | 2.290 | 23.709 | 16.069 | −0.57 | +0.35 | +0.324 | 4.894 |
| ATOM | 11 | O22 | MOL | 3 | 1.857 | 22.623 | 16.880 | +0.01 | −0.30 | −0.227 | 4.894 |
| ATOM | 12 | C20 | MOL | 3 | 0.831 | 21.875 | 16.172 | −0.48 | +0.15 | +0.113 | 4.894 |
| ATOM | 13 | C25 | MOL | 3 | 1.103 | 20.381 | 16.354 | −0.42 | +0.17 | +0.113 | 4.894 |
| ATOM | 14 | O28 | MOL | 3 | 2.319 | 20.245 | 17.065 | +0.06 | −0.54 | −0.368 | 4.894 |
| ATOM | 15 | P29 | MOL | 3 | 3.139 | 19.000 | 17.580 | −0.36 | +1.34 | +1.019 | 4.894 |
| ATOM | 16 | O30 | MOL | 3 | 2.331 | 17.779 | 17.305 | −0.01 | −0.31 | −0.255 | 4.894 |
| ATOM | 17 | O31 | MOL | 3 | 4.525 | 19.076 | 17.018 | +0.03 | −0.30 | −0.255 | 4.894 |
| ATOM | 18 | O32 | MOL | 3 | 3.038 | 19.377 | 19.127 | +0.02 | −0.84 | −0.510 | 4.894 |
| ATOM | 19 | P33 | MOL | 3 | 3.242 | 18.537 | 20.473 | −0.53 | +1.65 | +1.019 | 4.894 |
| ATOM | 20 | O35 | MOL | 3 | 1.840 | 18.427 | 20.950 | −0.37 | −0.65 | −0.255 | 4.894 |
| ATOM | 21 | O36 | MOL | 3 | 4.302 | 19.061 | 21.371 | +0.00 | −0.42 | −0.255 | 4.894 |
| ATOM | 22 | O34 | MOL | 3 | 3.814 | 17.105 | 19.979 | +0.06 | −0.43 | −0.368 | 4.894 |
| ATOM | 23 | C37 | MOL | 3 | 6.382 | 16.837 | 20.770 | −0.44 | +0.25 | +0.227 | 4.894 |
| ATOM | 24 | O39 | MOL | 3 | 6.312 | 15.640 | 20.403 | −0.03 | −0.22 | −0.227 | 4.894 |
| ATOM | 25 | C40 | MOL | 3 | 6.239 | 15.228 | 19.019 | −0.58 | +0.12 | +0.113 | 4.894 |
| ATOM | 26 | C50 | MOL | 3 | 6.528 | 13.739 | 19.061 | −0.72 | +0.13 | +0.113 | 4.894 |
| ATOM | 27 | O61 | MOL | 3 | 5.917 | 13.080 | 18.009 | −0.11 | −0.72 | −0.537 | 4.894 |
| ATOM | 28 | H61 | MOL | 3 | 5.433 | 13.743 | 17.493 | +0.04 | +0.55 | +0.424 | 4.894 |
| ATOM | 29 | C41 | MOL | 3 | 7.251 | 16.046 | 18.235 | −0.61 | +0.13 | +0.113 | 4.894 |
| ATOM | 30 | O48 | MOL | 3 | 7.145 | 15.794 | 16.854 | −0.17 | −0.49 | −0.537 | 4.894 |
| ATOM | 31 | H48 | MOL | 3 | 6.318 | 16.125 | 16.535 | −0.14 | +0.25 | +0.424 | 4.894 |
| ATOM | 32 | C42 | MOL | 3 | 6.972 | 17.526 | 18.438 | −0.50 | +0.14 | +0.113 | 4.894 |
| ATOM | 33 | O46 | MOL | 3 | 7.895 | 18.314 | 17.750 | +0.13 | −0.90 | −0.537 | 4.894 |
| ATOM | 34 | H46 | MOL | 3 | 8.587 | 17.740 | 17.445 | +0.04 | +0.65 | +0.424 | 4.894 |
| ATOM | 35 | C43 | MOL | 3 | 7.081 | 17.872 | 19.923 | −0.42 | +0.30 | +0.211 | 4.894 |
| ATOM | 36 | N51 | MOL | 3 | 6.629 | 19.201 | 20.212 | −0.23 | −0.99 | −0.650 | 4.894 |
| ATOM | 37 | H51 | MOL | 3 | 5.634 | 19.423 | 20.069 | +0.10 | +0.63 | +0.440 | 4.894 |
| ATOM | 38 | C53 | MOL | 3 | 7.453 | 20.179 | 20.665 | −0.48 | +0.69 | +0.396 | 4.894 |
| ATOM | 39 | O54 | MOL | 3 | 8.587 | 19.985 | 21.002 | −0.13 | −0.83 | −0.396 | 4.894 |
| ATOM | 40 | C55 | MOL | 3 | 6.833 | 21.559 | 20.683 | −0.42 | +0.00 | +0.000 | 4.894 |
| ATOM | 41 | C18 | MOL | 3 | 0.929 | 22.328 | 14.715 | −0.51 | +0.13 | +0.113 | 4.894 |
| ATOM | 42 | O23 | MOL | 3 | −0.114 | 23.264 | 14.431 | +0.01 | −0.61 | −0.537 | 4.894 |
| ATOM | 43 | H23 | MOL | 3 | −0.039 | 24.067 | 15.074 | +0.06 | +0.54 | +0.424 | 4.894 |
| ATOM | 44 | C14 | MOL | 3 | 2.264 | 23.107 | 14.661 | −0.58 | +0.11 | +0.113 | 4.894 |
| ATOM | 45 | O16 | MOL | 3 | 2.187 | 24.124 | 13.691 | −0.09 | −0.38 | −0.537 | 4.894 |
| ATOM | 46 | H16 | MOL | 3 | 1.765 | 23.747 | 12.830 | −0.01 | +0.32 | +0.424 | 4.894 |
| TER | | | | | | | | | | | |
| ENDMDL | | | | | | | | | | | |
| MODEL | 9 | | | | | | | | | | |
| USER | Run = 9 | | | | | | | | | | |
| USER | Cluster Rank = 4 | | | | | | | | | | |
| USER | Number of conformations in this cluster = 1 | | | | | | | | | | |
| USER | | | | | | | | | | | |
| USER | RMSD from reference structure = 5.679 A | | | | | | | | | | |
| USER | | | | | | | | | | | |
| USER | Estimated Free Energy of Binding = −7.35 kcal/mol [= (1) + (3)] | | | | | | | | | | |
| USER | Estimated Inhibition Constant, Ki = +4.09e−06 [Temperature = 298.15 K] | | | | | | | | | | |
| USER | | | | | | | | | | | |
| USER | Final Docked Energy = −9.65 kcal/mol [= (1) + (2)] | | | | | | | | | | |
| USER | | | | | | | | | | | |
| USER | (1) Final Intermolecular Energy = −12.02 kcal/mol | | | | | | | | | | |
| USER | (2) Final Internal Energy of Ligand = +2.37 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 1.020053 17.655890 10.395599 | | | | | | | | | | |
| USER | NEWDPF quat0 0.640783 0.642240 0.420625 112.634496 | | | | | | | | | | |
| USER | NEWDPF ndihe 15 | | | | | | | | | | |
| USER | NEWDPF dihe0 −177.74 −178.71 −111.09 −45.36 −180.00 −152.94 9.28 −35.96 | | | | | | | | | | |
| | −131.27 46.97 −162.47 −163.51 120.50 89.83 4.23 | | | | | | | | | | |
| USER | | | | | | | | | | | |
| USER | | | | Rank | x | y | z | vdW | Elec | q | RMS |
| ATOM | 1 | N1 | MOL | 4 | −1.166 | 19.176 | 9.972 | −0.37 | −0.28 | −0.211 | 5.679 |
| ATOM | 2 | C8 | MOL | 4 | −1.021 | 20.193 | 10.908 | −0.50 | +0.00 | +0.000 | 5.679 |
| ATOM | 3 | C6 | MOL | 4 | −1.379 | 20.060 | 12.194 | −0.66 | +0.00 | +0.000 | 5.679 |
| ATOM | 4 | C5 | MOL | 4 | −1.969 | 18.836 | 12.639 | −0.73 | +0.38 | +0.396 | 5.679 |
| ATOM | 5 | O11 | MOL | 4 | −2.344 | 18.561 | 13.779 | −0.11 | −0.29 | −0.396 | 5.679 |
| ATOM | 6 | N3 | MOL | 4 | −2.097 | 17.861 | 11.650 | −0.46 | −0.38 | −0.440 | 5.679 |
| ATOM | 7 | H3 | MOL | 4 | −2.507 | 16.960 | 11.936 | −0.16 | +0.21 | +0.440 | 5.679 |
| ATOM | 8 | C2 | MOL | 4 | −1.729 | 17.985 | 10.335 | −0.76 | +0.42 | +0.396 | 5.679 |
| ATOM | 9 | O10 | MOL | 4 | −1.884 | 17.067 | 9.527 | −0.25 | −0.30 | −0.396 | 5.679 |
| ATOM | 10 | C12 | MOL | 4 | −0.734 | 19.371 | 8.581 | −0.61 | +0.39 | +0.324 | 5.679 |
| ATOM | 11 | O22 | MOL | 4 | 0.686 | 19.460 | 8.612 | −0.05 | −0.33 | −0.227 | 5.679 |
| ATOM | 12 | C20 | MOL | 4 | 1.149 | 20.157 | 7.424 | −0.53 | +0.17 | +0.113 | 5.679 |
| ATOM | 13 | C25 | MOL | 4 | 2.241 | 21.147 | 7.832 | −0.46 | +0.19 | +0.113 | 5.679 |
| ATOM | 14 | O28 | MOL | 4 | 2.170 | 22.256 | 6.957 | −0.08 | −0.43 | −0.368 | 5.679 |
| ATOM | 15 | P29 | MOL | 4 | 2.753 | 22.541 | 5.520 | −0.45 | +1.04 | +1.019 | 5.679 |
| ATOM | 16 | O30 | MOL | 4 | 1.913 | 21.802 | 4.535 | +0.06 | −0.25 | −0.255 | 5.679 |
| ATOM | 17 | O31 | MOL | 4 | 2.908 | 24.020 | 5.352 | +0.06 | −0.22 | −0.255 | 5.679 |
| ATOM | 18 | O32 | MOL | 4 | 4.125 | 21.760 | 5.752 | +0.01 | −0.69 | −0.510 | 5.679 |
| ATOM | 19 | P33 | MOL | 4 | 5.541 | 21.792 | 5.009 | −0.30 | +1.15 | +1.019 | 5.679 |
| ATOM | 20 | O35 | MOL | 4 | 6.226 | 22.875 | 5.759 | +0.10 | −0.22 | −0.255 | 5.679 |
| ATOM | 21 | O36 | MOL | 4 | 5.458 | 21.842 | 3.527 | +0.17 | −0.23 | −0.255 | 5.679 |
| ATOM | 22 | O34 | MOL | 4 | 6.199 | 20.346 | 5.320 | −0.09 | −0.63 | −0.368 | 5.679 |
| ATOM | 23 | C37 | MOL | 4 | 8.822 | 20.392 | 4.680 | −0.14 | +0.20 | +0.227 | 5.679 |
| ATOM | 24 | O39 | MOL | 4 | 8.992 | 21.150 | 5.665 | +0.16 | −0.18 | −0.227 | 5.679 |
| ATOM | 25 | C40 | MOL | 4 | 8.888 | 20.716 | 7.039 | −0.36 | +0.09 | +0.113 | 5.679 |
| ATOM | 26 | C50 | MOL | 4 | 9.510 | 21.850 | 7.833 | −0.44 | +0.06 | +0.113 | 5.679 |
| ATOM | 27 | O61 | MOL | 4 | 9.374 | 21.642 | 9.194 | −0.05 | −0.14 | −0.537 | 5.679 |
| ATOM | 28 | H61 | MOL | 4 | 8.823 | 20.853 | 9.315 | −0.33 | −0.03 | +0.424 | 5.679 |
| ATOM | 29 | C41 | MOL | 4 | 9.612 | 19.386 | 7.165 | −0.42 | +0.11 | +0.113 | 5.679 |
| ATOM | 30 | O48 | MOL | 4 | 9.440 | 18.835 | 8.448 | −0.25 | −0.58 | −0.537 | 5.679 |
| ATOM | 31 | H48 | MOL | 4 | 8.522 | 18.665 | 8.598 | −0.29 | +0.49 | +0.424 | 5.679 |
| ATOM | 32 | C42 | MOL | 4 | 9.025 | 18.395 | 6.174 | −0.31 | +0.15 | +0.113 | 5.679 |
| ATOM | 33 | O46 | MOL | 4 | 9.679 | 17.164 | 6.242 | +0.06 | −0.72 | −0.537 | 5.679 |
| ATOM | 34 | H46 | MOL | 4 | 10.274 | 17.199 | 6.980 | +0.08 | +0.52 | +0.424 | 5.679 |
| ATOM | 35 | C43 | MOL | 4 | 9.189 | 18.930 | 4.751 | −0.21 | +0.22 | +0.211 | 5.679 |
| ATOM | 36 | N51 | MOL | 4 | 8.476 | 18.148 | 3.786 | −0.16 | −0.76 | −0.650 | 5.679 |
| ATOM | 37 | H51 | MOL | 4 | 9.011 | 17.518 | 3.171 | +0.11 | +0.42 | +0.440 | 5.679 |
| ATOM | 38 | C53 | MOL | 4 | 7.127 | 18.198 | 3.644 | −0.23 | +0.64 | +0.396 | 5.679 |
| ATOM | 39 | O54 | MOL | 4 | 6.404 | 18.780 | 4.402 | −0.24 | −0.92 | −0.396 | 5.679 |
| ATOM | 40 | C55 | MOL | 4 | 6.601 | 17.477 | 2.422 | −0.47 | +0.00 | +0.000 | 5.679 |
| ATOM | 41 | C18 | MOL | 4 | −0.090 | 20.840 | 6.844 | −0.59 | +0.12 | +0.113 | 5.679 |
| ATOM | 42 | O23 | MOL | 4 | −0.579 | 20.085 | 5.731 | −0.08 | −0.57 | −0.537 | 5.679 |
| ATOM | 43 | H23 | MOL | 4 | −1.307 | 20.627 | 5.242 | +0.04 | +0.33 | +0.424 | 5.679 |
| ATOM | 44 | C14 | MOL | 4 | −1.141 | 20.715 | 7.972 | −0.67 | +0.13 | +0.113 | 5.679 |
| ATOM | 45 | O16 | MOL | 4 | −2.432 | 20.634 | 7.417 | −0.19 | −0.57 | −0.537 | 5.679 |
| ATOM | 46 | H16 | MOL | 4 | −2.463 | 19.870 | 6.727 | +0.06 | +0.37 | +0.424 | 5.679 |
| TER | | | | | | | | | | | |
| ENDMDL | | | | | | | | | | | |
| MODEL | 5 | | | | | | | | | | |
| USER | Run = 5 | | | | | | | | | | |
| USER | Cluster Rank = 5 | | | | | | | | | | |
| USER | Number of conformations in this cluster = 1 | | | | | | | | | | |
| USER | | | | | | | | | | | |
| USER | RMSD from reference structure = 7.334 A | | | | | | | | | | |
| USER | | | | | | | | | | | |
| USER | Estimated Free Energy of Binding = −5.24 kcal/mol [= (1) + (3)] | | | | | | | | | | |
| USER | Estimated Inhibition Constant, Ki = +1.43e−04 [Temperature = 298.15 K] | | | | | | | | | | |
| USER | | | | | | | | | | | |
| USER | Final Docked Energy = −9.02 kcal/mol [= (1) + (2)] | | | | | | | | | | |
| USER | | | | | | | | | | | |
| USER | (1) Final Intermolecular Energy = −9.91 kcal/mol | | | | | | | | | | |
| USER | (2) Final Internal Energy of Ligand = +0.89 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 4.258166 20.961412 22.698509 | | | | | | | | | | |
| USER | NEWDPF quat0 0.940329 −0.334487 −0.062448 −84.022096 | | | | | | | | | | |
| USER | NEWDPF ndihe 15 | | | | | | | | | | |
| USER | NEWDPF dihe0 −23.42 76.46 −179.77 −58.39 −10.37 −170.73 176.51 −46.58 | | | | | | | | | | |
| | 124.47 36.92 −166.07 71.70 143.39 −62.34 −6.60 | | | | | | | | | | |
| USER | | | | | | | | | | | |
| USER | | | | Rank | x | y | z | vdW | Elec | q | RMS |
| ATOM | 1 | N1 | MOL | 5 | 4.800 | 18.329 | 22.479 | −0.32 | −0.30 | −0.211 | 7.334 |
| ATOM | 2 | C8 | MOL | 5 | 5.068 | 18.181 | 21.123 | −0.38 | +0.00 | +0.000 | 7.334 |
| ATOM | 3 | C6 | MOL | 5 | 4.107 | 18.047 | 20.197 | −0.37 | +0.00 | +0.000 | 7.334 |
| ATOM | 4 | C5 | MOL | 5 | 2.736 | 18.020 | 20.603 | −0.61 | +0.66 | +0.396 | 7.334 |
| ATOM | 5 | O11 | MOL | 5 | 1.747 | 17.909 | 19.879 | +0.01 | −0.86 | −0.396 | 7.334 |
| ATOM | 6 | N3 | MOL | 5 | 2.534 | 18.162 | 21.976 | −0.56 | −0.73 | −0.440 | 7.334 |
| ATOM | 7 | H3 | MOL | 5 | 1.558 | 18.160 | 22.305 | −0.28 | +0.82 | +0.440 | 7.334 |
| ATOM | 8 | C2 | MOL | 5 | 3.509 | 18.305 | 22.930 | −0.61 | +0.57 | +0.396 | 7.334 |
| ATOM | 9 | O10 | MOL | 5 | 3.237 | 18.421 | 24.126 | +0.25 | −0.53 | −0.396 | 7.334 |
| ATOM | 10 | C12 | MOL | 5 | 5.893 | 18.496 | 23.446 | −0.57 | +0.51 | +0.324 | 7.334 |
| ATOM | 11 | O22 | MOL | 5 | 6.629 | 19.640 | 23.024 | −0.20 | −0.45 | −0.227 | 7.334 |
| ATOM | 12 | C20 | MOL | 5 | 7.973 | 19.573 | 23.571 | −0.65 | +0.20 | +0.113 | 7.334 |
| ATOM | 13 | C25 | MOL | 5 | 8.965 | 19.977 | 22.480 | −0.25 | +0.22 | +0.113 | 7.334 |
| ATOM | 14 | O28 | MOL | 5 | 8.646 | 21.293 | 22.070 | −0.12 | −0.71 | −0.368 | 7.334 |
| ATOM | 15 | P29 | MOL | 5 | 7.364 | 21.945 | 21.423 | −0.49 | +1.74 | +1.019 | 7.334 |
| ATOM | 16 | O30 | MOL | 5 | 6.944 | 21.085 | 20.280 | −0.01 | −0.41 | −0.255 | 7.334 |
| ATOM | 17 | O31 | MOL | 5 | 6.370 | 22.221 | 22.507 | −0.25 | −0.60 | −0.255 | 7.334 |
| ATOM | 18 | O32 | MOL | 5 | 8.112 | 23.243 | 20.873 | +0.02 | −0.80 | −0.510 | 7.334 |
| ATOM | 19 | P33 | MOL | 5 | 7.594 | 24.656 | 20.330 | −0.27 | +1.18 | +1.019 | 7.334 |
| ATOM | 20 | O35 | MOL | 5 | 7.288 | 25.352 | 21.606 | +0.03 | −0.33 | −0.255 | 7.334 |
| ATOM | 21 | O36 | MOL | 5 | 6.568 | 24.566 | 19.260 | +0.09 | −0.23 | −0.255 | 7.334 |
| ATOM | 22 | O34 | MOL | 5 | 8.880 | 25.307 | 19.592 | +0.16 | −0.42 | −0.368 | 7.334 |
| ATOM | 23 | C37 | MOL | 5 | 8.911 | 24.554 | 17.000 | −0.40 | +0.30 | +0.227 | 7.334 |
| ATOM | 24 | O39 | MOL | 5 | 7.790 | 25.104 | 16.884 | −0.05 | −0.29 | −0.227 | 7.334 |
| ATOM | 25 | C40 | MOL | 5 | 7.590 | 26.535 | 16.917 | −0.27 | +0.11 | +0.113 | 7.334 |
| ATOM | 26 | C50 | MOL | 5 | 6.179 | 26.724 | 16.392 | +0.12 | +0.11 | +0.113 | 7.334 |
| ATOM | 27 | O61 | MOL | 5 | 5.638 | 27.921 | 16.828 | −0.04 | −0.38 | −0.537 | 7.334 |
| ATOM | 28 | H61 | MOL | 5 | 5.034 | 28.235 | 16.138 | −0.12 | +0.22 | +0.424 | 7.334 |
| ATOM | 29 | C41 | MOL | 5 | 8.672 | 27.177 | 16.066 | −0.22 | +0.09 | +0.113 | 7.334 |
| ATOM | 30 | O48 | MOL | 5 | 8.623 | 28.580 | 16.158 | +0.16 | −0.35 | −0.537 | 7.334 |
| ATOM | 31 | H48 | MOL | 5 | 7.723 | 28.868 | 16.118 | +0.10 | +0.28 | +0.424 | 7.334 |
| ATOM | 32 | C42 | MOL | 5 | 10.038 | 26.747 | 16.574 | −0.18 | +0.10 | +0.113 | 7.334 |
| ATOM | 33 | O46 | MOL | 5 | 11.062 | 27.299 | 15.804 | +0.16 | −0.39 | −0.537 | 7.334 |
| ATOM | 34 | H46 | MOL | 5 | 10.956 | 26.976 | 14.918 | +0.10 | +0.30 | +0.424 | 7.334 |
| ATOM | 35 | C43 | MOL | 5 | 10.163 | 25.226 | 16.492 | −0.36 | +0.24 | +0.211 | 7.334 |
| ATOM | 36 | N51 | MOL | 5 | 11.341 | 24.736 | 17.145 | −0.22 | −0.86 | −0.650 | 7.334 |
| ATOM | 37 | H51 | MOL | 5 | 11.444 | 24.896 | 18.157 | +0.10 | +0.57 | +0.440 | 7.334 |
| ATOM | 38 | C53 | MOL | 5 | 12.326 | 24.071 | 16.491 | −0.39 | +0.61 | +0.396 | 7.334 |
| ATOM | 39 | O54 | MOL | 5 | 12.235 | 23.706 | 15.353 | −0.13 | −0.62 | −0.396 | 7.334 |
| ATOM | 40 | C55 | MOL | 5 | 13.579 | 23.848 | 17.309 | −0.48 | +0.00 | +0.000 | 7.334 |
| ATOM | 41 | C18 | MOL | 5 | 8.153 | 18.127 | 24.036 | −0.64 | +0.13 | +0.113 | 7.334 |
| ATOM | 42 | O23 | MOL | 5 | 7.996 | 18.055 | 25.456 | −0.14 | −0.60 | −0.537 | 7.334 |
| ATOM | 43 | H23 | MOL | 5 | 8.064 | 17.072 | 25.759 | −0.24 | +0.23 | +0.424 | 7.334 |
| ATOM | 44 | C14 | MOL | 5 | 6.948 | 17.387 | 23.410 | −0.59 | +0.12 | +0.113 | 7.334 |
| ATOM | 45 | O16 | MOL | 5 | 6.563 | 16.313 | 24.235 | −0.16 | −0.46 | −0.537 | 7.334 |
| ATOM | 46 | H16 | MOL | 5 | 6.374 | 15.484 | 23.654 | +0.05 | +0.34 | +0.424 | 7.334 |
| TER | | | | | | | | | | | |
| ENDMDL | | | | | | | | | | | |
| MODEL | 7 | | | | | | | | | | |
| USER | Run = 7 | | | | | | | | | | |
| USER | Cluster Rank = 6 | | | | | | | | | | |
| USER | Number of conformations in this cluster = 1 | | | | | | | | | | |
| USER | | | | | | | | | | | |
| USER | RMSD from reference structure = 5.935 A | | | | | | | | | | |
| USER | | | | | | | | | | | |
| USER | Estimated Free Energy of Binding = −4.64 kcal/mol [= (1) + (3)] | | | | | | | | | | |
| USER | Estimated Inhibition Constant, Ki = +3.98e−04 [Temperature = 298.15 K] | | | | | | | | | | |
| USER | | | | | | | | | | | |
| USER | Final Docked Energy = −8.40 kcal/mol [= (1) + (2)] | | | | | | | | | | |
| USER | | | | | | | | | | | |
| USER | (1) Final Intermolecular Energy = −9.31 kcal/mol | | | | | | | | | | |
| USER | (2) Final Internal Energy of Ligand = +0.91 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 0.728396 20.563911 17.361005 | | | | | | | | | | |
| USER | NEWDPF quat0 0.187393 −0.291625 −0.937997 −94.040358 | | | | | | | | | | |
| USER | NEWDPF ndihe 15 | | | | | | | | | | |
| USER | NEWDPF dihe0 −75.09 −100.25 −4.22 −6.61 175.81 38.81 179.10 100.24 88.37 | | | | | | | | | | |
| | −17.46 −111.35 35.87 −70.02 43.92 55.17 | | | | | | | | | | |
| USER | | | | | | | | | | | |
| USER | | | | Rank | x | y | z | vdW | Elec | q | RMS |
| ATOM | 1 | N1 | MOL | 6 | 0.966 | 20.417 | 14.679 | −0.22 | −0.29 | −0.211 | 5.935 |
| ATOM | 2 | C8 | MOL | 6 | −0.196 | 21.036 | 14.232 | −0.53 | +0.00 | +0.000 | 5.935 |
| ATOM | 3 | C6 | MOL | 6 | −1.376 | 20.404 | 14.149 | −0.63 | +0.00 | +0.000 | 5.935 |
| ATOM | 4 | C5 | MOL | 6 | −1.462 | 19.021 | 14.500 | −0.69 | +0.37 | +0.396 | 5.935 |
| ATOM | 5 | O11 | MOL | 6 | −2.466 | 18.309 | 14.482 | −0.18 | −0.27 | −0.396 | 5.935 |
| ATOM | 6 | N3 | MOL | 6 | −0.261 | 18.456 | 14.930 | −0.39 | −0.50 | −0.440 | 5.935 |
| ATOM | 7 | H3 | MOL | 6 | −0.287 | 17.464 | 15.204 | −0.36 | +0.26 | +0.440 | 5.935 |
| ATOM | 8 | C2 | MOL | 6 | 0.949 | 19.094 | 15.024 | −0.38 | +0.67 | +0.396 | 5.935 |
| ATOM | 9 | O10 | MOL | 6 | 1.963 | 18.512 | 15.412 | −0.22 | −0.61 | −0.396 | 5.935 |
| ATOM | 10 | C12 | MOL | 6 | 2.226 | 21.166 | 14.772 | −0.43 | +0.41 | +0.324 | 5.935 |
| ATOM | 11 | O22 | MOL | 6 | 2.609 | 21.136 | 16.143 | +0.09 | −0.29 | −0.227 | 5.935 |
| ATOM | 12 | C20 | MOL | 6 | 3.507 | 22.247 | 16.414 | −0.44 | +0.13 | +0.113 | 5.935 |
| ATOM | 13 | C25 | MOL | 6 | 3.106 | 22.885 | 17.745 | −0.25 | +0.12 | +0.113 | 5.935 |
| ATOM | 14 | O28 | MOL | 6 | 4.164 | 23.729 | 18.156 | +0.32 | −0.26 | −0.368 | 5.935 |
| ATOM | 15 | P29 | MOL | 6 | 4.540 | 25.227 | 17.837 | −0.48 | +0.83 | +1.019 | 5.935 |
| ATOM | 16 | O30 | MOL | 6 | 5.304 | 25.240 | 16.556 | +0.07 | −0.30 | −0.255 | 5.935 |
| ATOM | 17 | O31 | MOL | 6 | 3.302 | 26.062 | 17.933 | −0.07 | −0.21 | −0.255 | 5.935 |
| ATOM | 18 | O32 | MOL | 6 | 5.545 | 25.400 | 19.065 | +0.07 | −0.36 | −0.510 | 5.935 |
| ATOM | 19 | P33 | MOL | 6 | 5.477 | 26.241 | 20.423 | −0.26 | +0.73 | +1.019 | 5.935 |
| ATOM | 20 | O35 | MOL | 6 | 4.008 | 26.380 | 20.594 | −0.08 | −0.18 | −0.255 | 5.935 |
| ATOM | 21 | O36 | MOL | 6 | 6.352 | 27.440 | 20.449 | +0.16 | −0.20 | −0.255 | 5.935 |
| ATOM | 22 | O34 | MOL | 6 | 6.115 | 25.262 | 21.544 | −0.06 | −0.36 | −0.368 | 5.935 |
| ATOM | 23 | C37 | MOL | 6 | 8.545 | 24.417 | 20.727 | −0.27 | +0.31 | +0.227 | 5.935 |
| ATOM | 24 | O39 | MOL | 6 | 8.370 | 23.400 | 21.439 | +0.02 | −0.37 | −0.227 | 5.935 |
| ATOM | 25 | C40 | MOL | 6 | 7.421 | 22.356 | 21.127 | −0.44 | +0.18 | +0.113 | 5.935 |
| ATOM | 26 | C50 | MOL | 6 | 7.815 | 21.213 | 22.044 | −0.54 | +0.21 | +0.113 | 5.935 |
| ATOM | 27 | O61 | MOL | 6 | 8.812 | 21.596 | 22.923 | −0.22 | −1.09 | −0.537 | 5.935 |
| ATOM | 28 | H61 | MOL | 6 | 8.385 | 21.844 | 23.758 | +0.08 | +0.87 | +0.424 | 5.935 |
| ATOM | 29 | C41 | MOL | 6 | 7.538 | 22.046 | 19.645 | −0.14 | +0.20 | +0.113 | 5.935 |
| ATOM | 30 | O48 | MOL | 6 | 6.550 | 21.130 | 19.239 | −0.16 | −0.89 | −0.537 | 5.935 |
| ATOM | 31 | H48 | MOL | 6 | 6.221 | 21.376 | 18.387 | +0.07 | +0.70 | +0.424 | 5.935 |
| ATOM | 32 | C42 | MOL | 6 | 7.319 | 23.318 | 18.843 | −0.26 | +0.16 | +0.113 | 5.935 |
| ATOM | 33 | O46 | MOL | 6 | 7.441 | 23.081 | 17.474 | +0.07 | −0.89 | −0.537 | 5.935 |
| ATOM | 34 | H46 | MOL | 6 | 6.587 | 23.222 | 17.084 | +0.07 | +0.59 | +0.424 | 5.935 |
| ATOM | 35 | C43 | MOL | 6 | 8.371 | 24.358 | 19.230 | −0.26 | +0.28 | +0.211 | 5.935 |
| ATOM | 36 | N51 | MOL | 6 | 8.100 | 25.648 | 18.666 | −0.10 | −0.66 | −0.650 | 5.935 |
| ATOM | 37 | H51 | MOL | 6 | 7.249 | 26.145 | 18.964 | +0.11 | +0.39 | +0.440 | 5.935 |
| ATOM | 38 | C53 | MOL | 6 | 8.914 | 26.243 | 17.758 | −0.17 | +0.38 | +0.396 | 5.935 |
| ATOM | 39 | O54 | MOL | 6 | 9.995 | 25.818 | 17.460 | +0.15 | −0.41 | −0.396 | 5.935 |
| ATOM | 40 | C55 | MOL | 6 | 8.334 | 27.485 | 17.119 | −0.17 | +0.00 | +0.000 | 5.935 |
| ATOM | 41 | C18 | MOL | 6 | 3.351 | 23.196 | 15.225 | −0.53 | +0.11 | +0.113 | 5.935 |
| ATOM | 42 | O23 | MOL | 6 | 4.456 | 23.035 | 14.331 | +0.00 | −0.42 | −0.537 | 5.935 |
| ATOM | 43 | H23 | MOL | 6 | 4.842 | 23.960 | 14.093 | −0.23 | +0.16 | +0.424 | 5.935 |
| ATOM | 44 | C14 | MOL | 6 | 2.096 | 22.666 | 14.493 | −0.55 | +0.11 | +0.113 | 5.935 |
| ATOM | 45 | O16 | MOL | 6 | 2.202 | 22.921 | 13.113 | −0.15 | −0.45 | −0.537 | 5.935 |
| ATOM | 46 | H16 | MOL | 6 | 3.120 | 22.600 | 12.773 | −0.20 | +0.34 | +0.424 | 5.935 |
| TER | | | | | | | | | | | |
| ENDMDL | | | | | | | | | | | |
| MODEL | 4 | | | | | | | | | | |
| USER | Run = 4 | | | | | | | | | | |
| USER | Cluster Rank = 7 | | | | | | | | | | |
| USER | Number of conformations in this cluster = 1 | | | | | | | | | | |
| USER | | | | | | | | | | | |
| USER | RMSD from reference structure = 7.581 A | | | | | | | | | | |
| USER | | | | | | | | | | | |
| USER | Estimated Free Energy of Binding = −3.96 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −8.34 kcal/mol [= (1) + (2)] | | | | | | | | | | |
| USER | | | | | | | | | | | |
| USER | (1) Final Intermolecular Energy = −8.63 kcal/mol | | | | | | | | | | |
| USER | (2) Final Internal Energy of Ligand = +0.29 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | MEWDPF tran0 0.687993 22.244981 19.074452 | | | | | | | | | | |
| USER | NEWDPF quat0 −0.185683 0.632655 0.751844 15.538127 | | | | | | | | | | |
| USER | NEWDPF ndihe 15 | | | | | | | | | | |
| USER | NEWDPF dihe0 16.30 169.45 −109.54 −46.75 145.39 −176.15 179.92 100.54 −132.69 | | | | | | | | | | |
| | −69.08 171.23 −52.40 −1.52 −174.35 −13.13 | | | | | | | | | | |
| USER | | | | | | | | | | | |
| USER | | | | Rank | x | y | z | vdW | Elec | q | RMS |
| ATOM | 1 | N1 | MOL | 7 | 1.511 | 21.800 | 16.546 | −0.24 | −0.29 | −0.211 | 7.581 |
| ATOM | 2 | C8 | MOL | 7 | 2.013 | 23.038 | 16.162 | −0.51 | +0.00 | +0.000 | 7.581 |
| ATOM | 3 | C6 | MOL | 7 | 1.236 | 24.037 | 15.719 | −0.70 | +0.00 | +0.000 | 7.581 |
| ATOM | 4 | C5 | MOL | 7 | −0.174 | 23.833 | 15.596 | −0.78 | +0.55 | +0.396 | 7.581 |
| ATOM | 5 | O11 | MOL | 7 | −1.014 | 24.647 | 15.213 | −0.53 | −0.64 | −0.396 | 7.581 |
| ATOM | 6 | N3 | MOL | 7 | −0.613 | 22.568 | 15.987 | −0.45 | −0.59 | −0.440 | 7.581 |
| ATOM | 7 | H3 | MOL | 7 | −1.625 | 22.387 | 15.924 | +0.04 | +0.56 | +0.440 | 7.581 |
| ATOM | 8 | C2 | MOL | 7 | 0.173 | 21.541 | 16.446 | −0.55 | +0.60 | +0.396 | 7.581 |
| ATOM | 9 | O10 | MOL | 7 | −0.304 | 20.451 | 16.765 | −0.30 | −0.77 | −0.396 | 7.581 |
| ATOM | 10 | C12 | MOL | 7 | 2.409 | 20.749 | 17.047 | −0.39 | +0.46 | +0.324 | 7.581 |
| ATOM | 11 | O22 | MOL | 7 | 3.199 | 21.351 | 18.066 | +0.03 | −0.30 | −0.227 | 7.581 |
| ATOM | 12 | C20 | MOL | 7 | 4.421 | 20.585 | 18.239 | −0.35 | +0.15 | +0.113 | 7.581 |
| ATOM | 13 | C25 | MOL | 7 | 5.592 | 21.559 | 18.378 | −0.35 | +0.15 | +0.113 | 7.581 |
| ATOM | 14 | O28 | MOL | 7 | 5.547 | 22.449 | 17.279 | −0.02 | −0.47 | −0.368 | 7.581 |
| ATOM | 15 | P29 | MOL | 7 | 6.063 | 23.924 | 17.059 | −0.48 | +1.21 | +1.019 | 7.581 |
| ATOM | 16 | O30 | MOL | 7 | 5.550 | 24.392 | 15.740 | +0.05 | −0.36 | −0.255 | 7.581 |
| ATOM | 17 | O31 | MOL | 7 | 5.733 | 24.731 | 18.276 | +0.06 | −0.22 | −0.255 | 7.581 |
| ATOM | 18 | O32 | MOL | 7 | 7.604 | 23.532 | 16.931 | −0.04 | −0.77 | −0.510 | 7.581 |
| ATOM | 19 | P33 | MOL | 7 | 8.914 | 24.360 | 16.536 | −0.40 | +1.40 | +1.019 | 7.581 |
| ATOM | 20 | O35 | MOL | 7 | 8.329 | 25.413 | 15.668 | +0.10 | −0.38 | −0.255 | 7.581 |
| ATOM | 21 | O36 | MOL | 7 | 10.045 | 23.530 | 16.050 | +0.22 | −0.42 | −0.255 | 7.581 |
| ATOM | 22 | O34 | MOL | 7 | 9.447 | 24.974 | 17.937 | +0.10 | −0.46 | −0.368 | 7.581 |
| ATOM | 23 | C37 | MOL | 7 | 11.332 | 26.843 | 17.444 | −0.11 | +0.19 | +0.227 | 7.581 |
| ATOM | 24 | O39 | MOL | 7 | 11.805 | 26.584 | 18.575 | +0.21 | −0.20 | −0.227 | 7.581 |
| ATOM | 25 | C40 | MOL | 7 | 12.490 | 25.353 | 18.896 | −0.17 | +0.13 | +0.113 | 7.581 |
| ATOM | 26 | C50 | MOL | 7 | 13.172 | 25.642 | 20.221 | −0.10 | +0.11 | +0.113 | 7.581 |
| ATOM | 27 | O61 | MOL | 7 | 13.316 | 24.490 | 20.973 | +0.15 | −0.63 | −0.537 | 7.581 |
| ATOM | 28 | H61 | MOL | 7 | 13.635 | 24.753 | 21.850 | +0.11 | +0.45 | +0.424 | 7.581 |
| ATOM | 29 | C41 | MOL | 7 | 13.439 | 25.034 | 17.754 | −0.26 | +0.13 | +0.113 | 7.581 |
| ATOM | 30 | O48 | MOL | 7 | 14.044 | 23.777 | 17.933 | −0.02 | −0.77 | −0.537 | 7.581 |
| ATOM | 31 | H48 | MOL | 7 | 13.380 | 23.129 | 18.118 | +0.06 | +0.83 | +0.424 | 7.581 |
| ATOM | 32 | C42 | MOL | 7 | 12.659 | 24.969 | 16.451 | −0.40 | +0.13 | +0.113 | 7.581 |
| ATOM | 33 | O46 | MOL | 7 | 13.499 | 24.700 | 15.371 | +0.03 | −0.61 | −0.537 | 7.581 |
| ATOM | 34 | H46 | MOL | 7 | 14.354 | 24.485 | 15.722 | +0.07 | +0.52 | +0.424 | 7.581 |
| ATOM | 35 | C43 | MOL | 7 | 11.980 | 26.313 | 16.189 | −0.25 | +0.18 | +0.211 | 7.581 |
| ATOM | 36 | N51 | MOL | 7 | 11.063 | 26.259 | 15.089 | −0.23 | −0.53 | −0.650 | 7.581 |
| ATOM | 37 | H51 | MOL | 7 | 10.223 | 25.670 | 15.178 | +0.09 | +0.43 | +0.440 | 7.581 |
| ATOM | 38 | C53 | MOL | 7 | 11.250 | 26.949 | 13.936 | −0.38 | +0.23 | +0.396 | 7.581 |
| ATOM | 39 | O54 | MOL | 7 | 12.110 | 27.772 | 13.790 | +0.02 | −0.20 | −0.396 | 7.581 |
| ATOM | 40 | C55 | MOL | 7 | 10.297 | 26.576 | 12.822 | −0.42 | +0.00 | +0.000 | 7.581 |
| ATOM | 41 | C18 | MOL | 7 | 4.525 | 19.697 | 16.998 | −0.39 | +0.14 | +0.113 | 7.581 |
| ATOM | 42 | O23 | MOL | 7 | 4.112 | 18.367 | 17.322 | +0.05 | −0.58 | −0.537 | 7.581 |
| ATOM | 43 | H23 | MOL | 7 | 4.317 | 17.741 | 16.528 | −0.07 | +0.32 | +0.424 | 7.581 |
| ATOM | 44 | C14 | MOL | 7 | 3.462 | 20.280 | 16.040 | −0.41 | +0.14 | +0.113 | 7.581 |
| ATOM | 45 | O16 | MOL | 7 | 2.942 | 19.259 | 15.222 | −0.14 | −0.76 | −0.537 | 7.581 |
| ATOM | 46 | H16 | MOL | 7 | 3.135 | 18.343 | 15.652 | −0.11 | +0.43 | +0.424 | 7.581 |
| TER | | | | | | | | | | | |
| ENDMDL | | | | | | | | | | | |
| MODEL | 8 | | | | | | | | | | |
| USER | Run = 8 | | | | | | | | | | |
| USER | Cluster Rank = 8 | | | | | | | | | | |
| USER | Number of conformations in this cluster = 1 | | | | | | | | | | |
| USER | | | | | | | | | | | |
| USER | RMSD from reference structure = 6.030 A | | | | | | | | | | |
| USER | | | | | | | | | | | |
| USER | Estimated Free Energy of Binding = −4.59 kcal/mol [= (1) + (3)] | | | | | | | | | | |
| USER | Estimated Inhibition Constant, Ki = +4.31e−04 [Temperature = 298.15 K] | | | | | | | | | | |
| USER | | | | | | | | | | | |
| USER | Final Docked Energy = −8.23 kcal/mol [= (1) + (2)] | | | | | | | | | | |
| USER | | | | | | | | | | | |
| USER | (1) Final Intermolecular Energy = −9.26 kcal/mol | | | | | | | | | | |
| USER | (2) Final Internal Energy of Ligand = +1.03 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 −2.524830 21.682421 9.725723 | | | | | | | | | | |
| USER | NEWDPF quat0 −0.593757 0.591535 0.545472 −179.267390 | | | | | | | | | | |
| USER | NEWDPF ndihe 15 | | | | | | | | | | |
| USER | NEWDPF dihe0 119.04 86.54 −179.91 −10.45 161.02 −165.63 135.14 34.90 −64.03 | | | | | | | | | | |
| | 173.46 −62.67 57.63 −14.25 22.76 −179.97 | | | | | | | | | | |
| USER | | | | | | | | | | | |
| USER | | | | Rank | x | y | z | vdW | Elec | q | RMS |
| ATOM | 1 | N1 | MOL | 8 | −0.938 | 19.505 | 9.614 | −0.36 | −0.28 | −0.211 | 6.030 |
| ATOM | 2 | C8 | MOL | 8 | −1.812 | 18.470 | 9.922 | −0.72 | +0.00 | +0.000 | 6.030 |
| ATOM | 3 | C6 | MOL | 8 | −2.148 | 18.144 | 11.180 | −0.71 | +0.00 | +0.000 | 6.030 |
| ATOM | 4 | C5 | MOL | 8 | −1.567 | 18.861 | 12.272 | −0.69 | +0.41 | +0.396 | 6.030 |
| ATOM | 5 | O11 | MOL | 8 | −1.775 | 18.686 | 13.472 | −0.07 | −0.31 | −0.396 | 6.030 |
| ATOM | 6 | N3 | MOL | 8 | −0.690 | 19.880 | 11.897 | −0.36 | −0.56 | −0.440 | 6.030 |
| ATOM | 7 | H3 | MOL | 8 | −0.262 | 20.426 | 12.657 | +0.08 | +0.55 | +0.440 | 6.030 |
| ATOM | 8 | C2 | MOL | 8 | −0.345 | 20.223 | 10.615 | −0.54 | +0.52 | +0.396 | 6.030 |
| ATOM | 9 | O10 | MOL | 8 | 0.444 | 21.139 | 10.378 | +0.21 | −0.50 | −0.396 | 6.030 |
| ATOM | 10 | C12 | MOL | 8 | −0.626 | 19.832 | 8.216 | −0.61 | +0.38 | +0.324 | 6.030 |
| ATOM | 11 | O22 | MOL | 8 | 0.764 | 19.576 | 8.046 | −0.04 | −0.34 | −0.227 | 6.030 |
| ATOM | 12 | C20 | MOL | 8 | 1.256 | 20.341 | 6.913 | −0.52 | +0.16 | +0.113 | 6.030 |
| ATOM | 13 | C25 | MOL | 8 | 2.605 | 20.957 | 7.289 | −0.30 | +0.21 | +0.113 | 6.030 |
| ATOM | 14 | O28 | MOL | 8 | 3.419 | 20.958 | 6.132 | +0.06 | −0.67 | −0.368 | 6.030 |
| ATOM | 15 | P29 | MOL | 8 | 4.457 | 21.986 | 5.539 | −0.41 | +1.25 | +1.019 | 6.030 |
| ATOM | 16 | O30 | MOL | 8 | 3.723 | 23.245 | 5.227 | +0.08 | −0.24 | −0.255 | 6.030 |
| ATOM | 17 | O31 | MOL | 8 | 5.635 | 22.061 | 6.461 | −0.07 | −0.32 | −0.255 | 6.030 |
| ATOM | 18 | O32 | MOL | 8 | 4.730 | 21.173 | 4.193 | +0.10 | −0.60 | −0.510 | 6.030 |
| ATOM | 19 | P33 | MOL | 8 | 5.322 | 19.718 | 3.895 | −0.41 | +1.58 | +1.019 | 6.030 |
| ATOM | 20 | O35 | MOL | 8 | 6.067 | 19.970 | 2.636 | +0.14 | −0.25 | −0.255 | 6.030 |
| ATOM | 21 | O36 | MOL | 8 | 4.325 | 18.620 | 3.970 | −0.15 | −0.52 | −0.255 | 6.030 |
| ATOM | 22 | O34 | MOL | 8 | 6.327 | 19.424 | 5.131 | −0.21 | −0.83 | −0.368 | 6.030 |
| ATOM | 23 | C37 | MOL | 8 | 8.866 | 19.936 | 4.370 | −0.14 | +0.21 | +0.227 | 6.030 |
| ATOM | 24 | O39 | MOL | 8 | 9.052 | 20.418 | 5.512 | +0.15 | −0.20 | −0.227 | 6.030 |
| ATOM | 25 | C40 | MOL | 8 | 9.113 | 19.625 | 6.719 | −0.39 | +0.11 | +0.113 | 6.030 |
| ATOM | 26 | C50 | MOL | 8 | 9.695 | 20.569 | 7.754 | −0.44 | +0.08 | +0.113 | 6.030 |
| ATOM | 27 | O61 | MOL | 8 | 9.448 | 20.118 | 9.039 | +0.01 | −0.26 | −0.537 | 6.030 |
| ATOM | 28 | H61 | MOL | 8 | 8.496 | 20.205 | 9.199 | −0.39 | −0.09 | +0.424 | 6.030 |
| ATOM | 29 | C41 | MOL | 8 | 9.965 | 18.401 | 6.430 | −0.35 | +0.12 | +0.113 | 6.030 |
| ATOM | 30 | O48 | MOL | 8 | 9.960 | 17.512 | 7.520 | +0.01 | −0.70 | −0.537 | 6.030 |
| ATOM | 31 | H48 | MOL | 8 | 9.891 | 18.001 | 8.327 | +0.05 | +0.55 | +0.424 | 6.030 |
| ATOM | 32 | C42 | MOL | 8 | 9.387 | 17.650 | 5.243 | −0.22 | +0.14 | +0.113 | 6.030 |
| ATOM | 33 | O46 | MOL | 8 | 10.155 | 16.528 | 4.930 | +0.07 | −0.58 | −0.537 | 6.030 |
| ATOM | 34 | H46 | MOL | 8 | 11.022 | 16.834 | 4.695 | +0.11 | +0.39 | +0.424 | 6.030 |
| ATOM | 35 | C43 | MOL | 8 | 9.372 | 18.560 | 4.015 | −0.18 | +0.20 | +0.211 | 6.030 |
| ATOM | 36 | N51 | MOL | 8 | 8.651 | 17.987 | 2.916 | −0.11 | −0.61 | −0.650 | 6.030 |
| ATOM | 37 | H51 | MOL | 8 | 7.969 | 17.241 | 3.111 | +0.09 | +0.51 | +0.440 | 6.030 |
| ATOM | 38 | C53 | MOL | 8 | 8.827 | 18.384 | 1.630 | −0.11 | +0.28 | +0.396 | 6.030 |
| ATOM | 39 | O54 | MOL | 8 | 9.481 | 19.338 | 1.316 | +0.22 | −0.24 | −0.396 | 6.030 |
| ATOM | 40 | C55 | MOL | 8 | 8.148 | 17.501 | 0.606 | −0.18 | +0.00 | +0.000 | 6.030 |
| ATOM | 41 | C18 | MOL | 8 | 0.179 | 21.389 | 6.628 | −0.58 | +0.11 | +0.113 | 6.030 |
| ATOM | 42 | O23 | MOL | 8 | −0.607 | 20.978 | 5.506 | −0.06 | −0.46 | −0.537 | 6.030 |
| ATOM | 43 | H23 | MOL | 8 | −0.747 | 21.780 | 4.873 | +0.07 | +0.26 | +0.424 | 6.030 |
| ATOM | 44 | C14 | MOL | 8 | −0.737 | 21.320 | 7.872 | −0.61 | +0.12 | +0.113 | 6.030 |
| ATOM | 45 | O16 | MOL | 8 | −2.059 | 21.645 | 7.512 | −0.24 | −0.51 | −0.537 | 6.030 |
| ATOM | 46 | H16 | MOL | 8 | −2.254 | 21.293 | 6.564 | +0.06 | +0.29 | +0.424 | 6.030 |
| TER | | | | | | | | | | | |
| ENDMDL | | | | | | | | | | | |
| MODEL | 2 | | | | | | | | | | |
| USER | Run = 2 | | | | | | | | | | |
| USER | Cluster Rank = 9 | | | | | | | | | | |
| USER | Number of conformations in this cluster = 1 | | | | | | | | | | |
| USER | | | | | | | | | | | |
| USER | RMSD from reference structure = 6.255 A | | | | | | | | | | |
| USER | | | | | | | | | | | |
| USER | Estimated Free Energy of Binding = −3.12 kcal/mol [= (1) + (3)] | | | | | | | | | | |
| USER | Estimated Inhibition Constant, Ki = +0.01 [Temperature = 298.15 K] | | | | | | | | | | |
| USER | | | | | | | | | | | |
| USER | Final Docked Energy = −6.79 kcal/mol [= (1) + (2)] | | | | | | | | | | |
| USER | | | | | | | | | | | |
| USER | (1) Final Intermolecular Energy = −7.79 kcal/mol | | | | | | | | | | |
| USER | (2) Final Internal Energy of Ligand = +1.00 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 4.648066 21.814162 20.321720 | | | | | | | | | | |
| USER | NEWDPF quat0 0.730047 −0.671498 0.126969 −68.129830 | | | | | | | | | | |
| USER | NEWDPF ndihe 15 | | | | | | | | | | |
| USER | NEWDPF dihe0 146.22 −18.60 168.63 −55.42 −87.95 76.92 159.06 −61.03 | | | | | | | | | | |
| | 179.91 −154.80 37.43 72.09 −176.34 −24.18 16.94 | | | | | | | | | | |
| USER | | | | | | | | | | | |
| USER | | | | Rank | x | y | z | vdW | Elec | q | RMS |
| ATOM | 1 | N1 | MOL | 9 | 3.935 | 19.496 | 19.144 | −0.21 | −0.30 | −0.211 | 6.255 |
| ATOM | 2 | C8 | MOL | 9 | 4.140 | 19.760 | 17.795 | −0.33 | +0.00 | +0.000 | 6.255 |
| ATOM | 3 | C6 | MOL | 9 | 3.247 | 20.407 | 17.031 | −0.36 | +0.00 | +0.000 | 6.255 |
| ATOM | 4 | C5 | MOL | 9 | 2.005 | 20.826 | 17.601 | −0.44 | +0.65 | +0.396 | 6.255 |
| ATOM | 5 | O11 | MOL | 9 | 1.093 | 21.430 | 17.037 | −0.07 | −0.64 | −0.396 | 6.255 |
| ATOM | 6 | N3 | MOL | 9 | 1.856 | 20.523 | 18.955 | −0.26 | −1.00 | −0.440 | 6.255 |
| ATOM | 7 | H3 | MOL | 9 | 0.979 | 20.821 | 19.403 | +0.09 | +1.46 | +0.440 | 6.255 |
| ATOM | 8 | C2 | MOL | 9 | 2.765 | 19.867 | 19.745 | −0.44 | +0.77 | +0.396 | 6.255 |
| ATOM | 9 | O10 | MOL | 9 | 2.545 | 19.638 | 20.935 | −0.46 | −0.92 | −0.396 | 6.255 |
| ATOM | 10 | C12 | MOL | 9 | 4.960 | 18.804 | 19.937 | −0.33 | +0.44 | +0.324 | 6.255 |
| ATOM | 11 | O22 | MOL | 9 | 5.924 | 19.789 | 20.292 | +0.05 | −0.34 | −0.227 | 6.255 |
| ATOM | 12 | C20 | MOL | 9 | 7.186 | 19.136 | 20.599 | −0.40 | +0.19 | +0.113 | 6.255 |
| ATOM | 13 | C25 | MOL | 9 | 8.319 | 19.937 | 19.955 | −0.13 | +0.23 | +0.113 | 6.255 |
| ATOM | 14 | O28 | MOL | 9 | 8.870 | 20.782 | 20.947 | −0.04 | −0.74 | −0.368 | 6.255 |
| ATOM | 15 | P29 | MOL | 9 | 8.328 | 21.337 | 22.320 | −0.55 | +1.94 | +1.019 | 6.255 |
| ATOM | 16 | O30 | MOL | 9 | 6.956 | 21.871 | 22.084 | −0.15 | −0.50 | −0.255 | 6.255 |
| ATOM | 17 | O31 | MOL | 9 | 8.509 | 20.283 | 23.367 | −0.16 | −0.48 | −0.255 | 6.255 |
| ATOM | 18 | O32 | MOL | 9 | 9.366 | 22.545 | 22.420 | −0.26 | −1.01 | −0.510 | 6.255 |
| ATOM | 19 | P33 | MOL | 9 | 9.985 | 23.547 | 21.338 | −0.37 | +1.70 | +1.019 | 6.255 |
| ATOM | 20 | O35 | MOL | 9 | 11.409 | 23.125 | 21.348 | +0.24 | −0.48 | −0.255 | 6.255 |
| ATOM | 21 | O36 | MOL | 9 | 9.624 | 24.973 | 21.541 | +0.14 | −0.34 | −0.255 | 6.255 |
| ATOM | 22 | O34 | MOL | 9 | 9.258 | 23.149 | 19.947 | −0.06 | −0.74 | −0.368 | 6.255 |
| ATOM | 23 | C37 | MOL | 9 | 8.864 | 25.371 | 18.465 | −0.20 | +0.25 | +0.227 | 6.255 |
| ATOM | 24 | O39 | MOL | 9 | 10.040 | 25.700 | 18.747 | +0.17 | −0.24 | −0.227 | 6.255 |
| ATOM | 25 | C40 | MOL | 9 | 11.209 | 25.030 | 18.222 | −0.26 | +0.14 | +0.113 | 6.255 |
| ATOM | 26 | C50 | MOL | 9 | 12.352 | 25.975 | 18.543 | −0.12 | +0.11 | +0.113 | 6.255 |
| ATOM | 27 | O61 | MOL | 9 | 12.253 | 26.462 | 19.834 | +0.22 | −0.48 | −0.537 | 6.255 |
| ATOM | 28 | H61 | MOL | 9 | 11.320 | 26.675 | 19.994 | +0.12 | +0.38 | +0.424 | 6.255 |
| ATOM | 29 | C41 | MOL | 9 | 10.984 | 24.792 | 16.739 | −0.38 | +0.14 | +0.113 | 6.255 |
| ATOM | 30 | O48 | MOL | 9 | 12.024 | 24.024 | 16.185 | −0.07 | −0.81 | −0.537 | 6.255 |
| ATOM | 31 | H48 | MOL | 9 | 12.267 | 24.380 | 15.343 | +0.05 | +0.54 | +0.424 | 6.255 |
| ATOM | 32 | C42 | MOL | 9 | 9.699 | 24.005 | 16.542 | −0.24 | +0.17 | +0.113 | 6.255 |
| ATOM | 33 | O46 | MOL | 9 | 9.443 | 23.783 | 15.188 | −0.39 | −0.92 | −0.537 | 6.255 |
| ATOM | 34 | H46 | MOL | 9 | 9.163 | 24.609 | 14.814 | +0.05 | +0.59 | +0.424 | 6.255 |
| ATOM | 35 | C43 | MOL | 9 | 8.520 | 24.791 | 17.115 | −0.37 | +0.27 | +0.211 | 6.255 |
| ATOM | 36 | N51 | MOL | 9 | 7.312 | 24.021 | 17.150 | −0.30 | −0.87 | −0.650 | 6.255 |
| ATOM | 37 | H51 | MOL | 9 | 7.358 | 23.050 | 17.489 | +0.06 | +0.72 | +0.440 | 6.255 |
| ATOM | 38 | C53 | MOL | 9 | 6.110 | 24.514 | 16.758 | −0.45 | +0.49 | +0.396 | 6.255 |
| ATOM | 39 | O54 | MOL | 9 | 5.923 | 25.668 | 16.491 | −0.02 | −0.50 | −0.396 | 6.255 |
| ATOM | 40 | C55 | MOL | 9 | 5.014 | 23.476 | 16.653 | −0.49 | +0.00 | +0.000 | 6.255 |
| ATOM | 41 | C18 | MOL | 9 | 7.060 | 17.720 | 20.035 | −0.43 | +0.16 | +0.113 | 6.255 |
| ATOM | 42 | O23 | MOL | 9 | 6.779 | 16.803 | 21.097 | −0.04 | −0.60 | −0.537 | 6.255 |
| ATOM | 43 | H23 | MOL | 9 | 6.691 | 15.850 | 20.712 | +0.08 | +0.42 | +0.424 | 6.255 |
| ATOM | 44 | C14 | MOL | 9 | 5.793 | 17.788 | 19.150 | −0.33 | +0.14 | +0.113 | 6.255 |
| ATOM | 45 | O16 | MOL | 9 | 5.153 | 16.534 | 19.131 | +0.09 | −0.55 | −0.537 | 6.255 |
| ATOM | 46 | H16 | MOL | 9 | 4.134 | 16.669 | 19.074 | +0.10 | +0.45 | +0.424 | 6.255 |
| TER | | | | | | | | | | | |
| ENDMDL | | | | | | | | | | | |
| MODEL | 3 | | | | | | | | | | |
| USER | Run = 3 | | | | | | | | | | |
| USER | Cluster Rank = 10 | | | | | | | | | | |
| USER | Number of conformations in this cluster = 1 | | | | | | | | | | |
| USER | | | | | | | | | | | |
| USER | RMSD from reference structure = 6.976 A | | | | | | | | | | |
| USER | | | | | | | | | | | |
| USER | Estimated Free Energy of Binding = −2.58 kcal/mol [= (1) + (3)] | | | | | | | | | | |
| USER | Estimated Inhibition Constant, Ki = +0.01 [Temperature = 298.15 K] | | | | | | | | | | |
| USER | | | | | | | | | | | |
| USER | Final Docked Energy = −5.75 kcal/mol [= (1) + (2)] | | | | | | | | | | |
| USER | | | | | | | | | | | |
| USER | (1) Final Intermolecular Energy = −7.25 kcal/mol | | | | | | | | | | |
| USER | (2) Final Internal Energy of Ligand = +1.50 kcal/mol | | | | | | | | | | |
| USER | (3) Torsional Free Energy = +4.67 kcal/mol | | | | | | | | | | |
| USER | | | | | | | | | | | |
| USER | | | | | | | | | | | |
| USER | DPF = mx.dpf | | | | | | | | | | |
| USER | NEWDPF move udpg_ts.pdbq | | | | | | | | | | |
| USER | NEWDPF about −1.182000 20.257999 9.670000 | | | | | | | | | | |
| USER | NEWDPF tran0 1.794389 24.856810 6.189465 | | | | | | | | | | |
| USER | NEWDPF quat0 −0.641019 0.660766 0.390492 108.131136 | | | | | | | | | | |
| USER | NEWDPF ndihe 15 | | | | | | | | | | |
| USER | NEWDPF dihe0 −168.84 26.97 161.52 −61.69 164.44 −153.50 −175.69 −33.16 | | | | | | | | | | |
| | 117.08 12.90 −159.50 122.88 −63.07 68.07 −67.91 | | | | | | | | | | |
| USER | | | | | | | | | | | |
| USER | | | | Rank | x | y | z | vdW | Elec | q | RMS |
| ATOM | 1 | N1 | MOL | 10 | 1.826 | 22.251 | 5.499 | −0.27 | −0.21 | −0.211 | 6.976 |
| ATOM | 2 | C8 | MOL | 10 | 0.906 | 22.196 | 4.459 | −0.45 | +0.00 | +0.000 | 6.976 |
| ATOM | 3 | C6 | MOL | 10 | −0.419 | 22.114 | 4.656 | −0.56 | +0.00 | +0.000 | 6.976 |
| ATOM | 4 | C5 | MOL | 10 | −0.929 | 22.045 | 5.990 | −0.58 | +0.27 | +0.396 | 6.976 |
| ATOM | 5 | O11 | MOL | 10 | −2.109 | 21.972 | 6.333 | −0.21 | −0.22 | −0.396 | 6.976 |
| ATOM | 6 | N3 | MOL | 10 | 0.045 | 22.094 | 6.987 | −0.38 | −0.40 | −0.440 | 6.976 |
| ATOM | 7 | H3 | MOL | 10 | −0.287 | 22.060 | 7.962 | +0.05 | +0.44 | +0.440 | 6.976 |
| ATOM | 8 | C2 | MOL | 10 | 1.400 | 22.183 | 6.796 | −0.61 | +0.42 | +0.396 | 6.976 |
| ATOM | 9 | O10 | MOL | 10 | 2.189 | 22.216 | 7.741 | −0.11 | −0.50 | −0.396 | 6.976 |
| ATOM | 10 | C12 | MOL | 10 | 3.264 | 22.365 | 5.222 | −0.40 | +0.34 | +0.324 | 6.976 |
| ATOM | 11 | O22 | MOL | 10 | 3.406 | 22.299 | 3.807 | +0.13 | −0.21 | −0.227 | 6.976 |
| ATOM | 12 | C20 | MOL | 10 | 4.759 | 21.880 | 3.480 | −0.20 | +0.10 | +0.113 | 6.976 |
| ATOM | 13 | C25 | MOL | 10 | 4.691 | 20.849 | 2.353 | −0.22 | +0.10 | +0.113 | 6.976 |
| ATOM | 14 | O28 | MOL | 10 | 5.979 | 20.284 | 2.204 | +0.16 | −0.32 | −0.368 | 6.976 |
| ATOM | 15 | P29 | MOL | 10 | 6.680 | 19.009 | 2.814 | −0.28 | +1.16 | +1.019 | 6.976 |
| ATOM | 16 | O30 | MOL | 10 | 5.677 | 18.298 | 3.656 | −0.26 | −0.55 | −0.255 | 6.976 |
| ATOM | 17 | O31 | MOL | 10 | 7.340 | 18.251 | 1.705 | +0.15 | −0.21 | −0.255 | 6.976 |
| ATOM | 18 | O32 | MOL | 10 | 7.691 | 19.818 | 3.746 | +0.15 | −0.55 | −0.510 | 6.976 |
| ATOM | 19 | P33 | MOL | 10 | 9.089 | 19.445 | 4.426 | −0.15 | +0.98 | +1.019 | 6.976 |
| ATOM | 20 | O35 | MOL | 10 | 9.868 | 19.004 | 3.241 | +0.21 | −0.20 | −0.255 | 6.976 |
| ATOM | 21 | O36 | MOL | 10 | 8.980 | 18.570 | 5.620 | +0.06 | −0.32 | −0.255 | 6.976 |
| ATOM | 22 | O34 | MOL | 10 | 9.647 | 20.848 | 5.013 | +0.20 | −0.28 | −0.368 | 6.976 |
| ATOM | 23 | C37 | MOL | 10 | 8.492 | 21.349 | 7.401 | −0.36 | +0.14 | +0.227 | 6.976 |
| ATOM | 24 | O39 | MOL | 10 | 9.215 | 20.443 | 7.878 | −0.01 | −0.15 | −0.227 | 6.976 |
| ATOM | 25 | C40 | MOL | 10 | 10.644 | 20.564 | 8.054 | −0.44 | +0.09 | +0.113 | 6.976 |
| ATOM | 26 | C50 | MOL | 10 | 11.005 | 19.404 | 8.964 | −0.59 | +0.11 | +0.113 | 6.976 |
| ATOM | 27 | O61 | MOL | 10 | 12.376 | 19.297 | 9.120 | −0.15 | −0.68 | −0.537 | 6.976 |
| ATOM | 28 | H61 | MOL | 10 | 12.782 | 20.034 | 8.637 | +0.09 | +0.45 | +0.424 | 6.976 |
| ATOM | 29 | C41 | MOL | 10 | 10.928 | 21.937 | 8.638 | −0.40 | +0.08 | +0.113 | 6.976 |
| ATOM | 30 | O48 | MOL | 10 | 12.312 | 22.180 | 8.713 | +0.01 | −0.41 | −0.537 | 6.976 |
| ATOM | 31 | H48 | MOL | 10 | 12.634 | 21.914 | 9.562 | +0.05 | +0.40 | +0.424 | 6.976 |
| ATOM | 32 | C42 | MOL | 10 | 10.339 | 23.005 | 7.731 | −0.34 | +0.07 | +0.113 | 6.976 |
| ATOM | 33 | O46 | MOL | 10 | 10.559 | 24.283 | 8.245 | +0.12 | −0.43 | −0.537 | 6.976 |
| ATOM | 34 | H46 | MOL | 10 | 9.906 | 24.435 | 8.916 | +0.07 | +0.32 | +0.424 | 6.976 |
| ATOM | 35 | C43 | MOL | 10 | 8.828 | 22.805 | 7.613 | −0.32 | +0.10 | +0.211 | 6.976 |
| ATOM | 36 | N51 | MOL | 10 | 8.240 | 23.646 | 6.613 | −0.15 | −0.38 | −0.650 | 6.976 |
| ATOM | 37 | H51 | MOL | 10 | 8.446 | 23.450 | 5.624 | +0.11 | +0.28 | +0.440 | 6.976 |
| ATOM | 38 | C53 | MOL | 10 | 7.424 | 24.689 | 6.911 | −0.33 | +0.22 | +0.396 | 6.976 |
| ATOM | 39 | O54 | MOL | 10 | 7.006 | 24.907 | 8.013 | −0.05 | −0.12 | −0.396 | 6.976 |
| ATOM | 40 | C55 | MOL | 10 | 7.101 | 25.584 | 5.736 | −0.20 | +0.00 | +0.000 | 6.976 |
| ATOM | 41 | C18 | MOL | 10 | 5.334 | 21.311 | 4.778 | −0.32 | +0.14 | +0.113 | 6.976 |
| ATOM | 42 | O23 | MOL | 10 | 6.201 | 22.275 | 5.383 | +0.12 | −0.52 | −0.537 | 6.976 |
| ATOM | 43 | H23 | MOL | 10 | 6.501 | 21.933 | 6.309 | +0.09 | +0.45 | +0.424 | 6.976 |
| ATOM | 44 | C14 | MOL | 10 | 4.099 | 21.173 | 5.698 | −0.42 | +0.18 | +0.113 | 6.976 |
| ATOM | 45 | O16 | MOL | 10 | 4.481 | 21.343 | 7.042 | −0.03 | −1.09 | −0.537 | 6.976 |
| ATOM | 46 | H16 | MOL | 10 | 5.195 | 22.083 | 7.105 | +0.05 | +0.60 | +0.424 | 6.976 |
| TER | | | | | | | | | | | |

TABLE 20
CLUSTERING HISTOGRAM

| | Lowest | | Mean | Num | | | | | | | |
|---|---|---|---|---|---|---|---|---|---|---|---|
| Cluster | Docked | | Docked | in | Histogram | | | | | | |
| Rank | Energy | Run | Energy | Clus | 5 | 10 | 15 | 20 | 25 | 30 | 35 |
| 1 | −4.88 | 62 | −4.88 | 1 | # | | | | | | |
| 2 | −3.49 | 27 | −3.49 | 1 | # | | | | | | |
| 3 | −3.31 | 44 | −3.31 | 1 | # | | | | | | |
| 4 | −3.10 | 87 | −3.10 | 1 | # | | | | | | |
| 5 | −2.97 | 63 | −2.97 | 1 | # | | | | | | |
| 6 | −2.93 | 49 | −2.93 | 1 | # | | | | | | |
| 7 | −2.64 | 47 | −2.64 | 1 | # | | | | | | |
| 8 | −2.39 | 32 | −2.39 | 1 | # | | | | | | |
| 9 | −2.23 | 28 | −2.23 | 1 | # | | | | | | |
| 10 | −2.08 | 82 | −2.08 | 1 | # | | | | | | |
| 11 | −2.05 | 84 | −2.05 | 1 | # | | | | | | |
| 12 | −2.03 | 55 | −2.03 | 1 | # | | | | | | |
| 13 | −1.88 | 99 | −1.88 | 1 | # | | | | | | |
| 14 | −1.75 | 8 | −1.75 | 1 | # | | | | | | |
| 15 | −1.75 | 88 | −1.75 | 1 | # | | | | | | |
| 16 | −1.68 | 58 | −1.68 | 1 | # | | | | | | |
| 17 | −1.66 | 34 | −1.66 | 1 | # | | | | | | |
| 18 | −0.87 | 77 | −0.87 | 1 | # | | | | | | |
| 19 | −0.82 | 29 | −0.82 | 1 | # | | | | | | |
| 20 | −0.67 | 38 | −0.67 | 1 | # | | | | | | |

LOWEST ENERGY DOCKED CONFORMATION from EACH CLUSTER
Residue number will be set to the conformation's cluster rank.

| MODEL | 62 | | | | | | | | | | |
|---|---|---|---|---|---|---|---|---|---|---|---|
| USER | Run = 62 | | | | | | | | | | |
| USER | Cluster Rank = 1 | | | | | | | | | | |
| USER | Number of conformations in this cluster = 1 | | | | | | | | | | |
| USER | | | | | | | | | | | |
| USER | RMSD from reference structure = 3.876 A | | | | | | | | | | |
| USER | | | | | | | | | | | |
| USER | Estimated Free Energy of Binding = −1.78 kcal/mol [= (1) + (3)] | | | | | | | | | | |
| USER | Estimated Inhibition Constant, Ki = +0.05 [Temperature = 298.15 K] | | | | | | | | | | |
| USER | | | | | | | | | | | |
| USER | Final Docked Energy = −4.88 kcal/mol [= (1) + (2)] | | | | | | | | | | |
| USER | | | | | | | | | | | |
| USER | (1) Final Intermolecular Energy = −6.76 kcal/mol | | | | | | | | | | |
| USER | (2) Final Internal Energy of Ligand = +1.88 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 4.544220 27.873043 21.501519 | | | | | | | | | | |
| USER | NEWDPF quat0 0.566684 0.230230 −0.791115 35.808387 | | | | | | | | | | |
| USER | NEWDPF ndihe 16 | | | | | | | | | | |
| USER | NEWDPF dihe0 −166.16 −114.38 178.16 −68.11 −15.89 −14.65 −9.73 −13.78 | | | | | | | | | | |
| | 88.51 −27.33 −13.36 65.20 29.45 −21.54 −14.43 0.01 | | | | | | | | | | |
| USER | | | | | | | | | | | |
| USER | | | | Rank | x | y | z | vdW | Elec | q | RMS |
| ATOM | 1 | C5 | MAN | 1 | 5.127 | 22.402 | 17.233 | −0.31 | +0.11 | +0.113 | 3.876 |
| ATOM | 2 | HO4 | MAN | 1 | 6.555 | 22.327 | 19.496 | +0.09 | +0.43 | +0.424 | 3.876 |
| ATOM | 3 | O4 | MAN | 1 | 6.139 | 21.437 | 19.185 | +0.05 | −0.54 | −0.537 | 3.876 |
| ATOM | 4 | O5 | MAN | 1 | 3.885 | 22.914 | 16.698 | +0.01 | −0.23 | −0.227 | 3.876 |
| ATOM | 5 | HO2 | MAN | 1 | 0.974 | 22.288 | 18.411 | −0.24 | −0.26 | +0.424 | 3.876 |
| ATOM | 6 | O2 | MAN | 1 | 1.945 | 21.960 | 18.501 | +0.07 | +0.00 | −0.537 | 3.876 |
| ATOM | 7 | C2 | MAN | 1 | 2.782 | 23.050 | 18.834 | −0.45 | +0.07 | +0.113 | 3.876 |
| ATOM | 8 | C1 | MAN | 1 | 3.180 | 23.798 | 17.573 | −0.43 | +0.20 | +0.227 | 3.876 |
| ATOM | 9 | C4 | MAN | 1 | 4.886 | 21.688 | 18.574 | −0.29 | +0.10 | +0.113 | 3.876 |
| ATOM | 10 | O3 | MAN | 1 | 3.673 | 21.775 | 20.662 | −0.09 | −0.63 | −0.537 | 3.876 |
| ATOM | 11 | HO3 | MAN | 1 | 3.200 | 22.380 | 21.348 | −0.04 | +0.54 | +0.424 | 3.876 |
| ATOM | 12 | C3 | MAN | 1 | 4.038 | 22.542 | 19.527 | −0.37 | +0.11 | +0.113 | 3.876 |
| ATOM | 13 | C6 | MAN | 1 | 5.715 | 21.472 | 16.185 | +0.11 | +0.09 | +0.113 | 3.876 |
| ATOM | 14 | O6 | MAN | 1 | 4.839 | 20.388 | 15.903 | +0.09 | −0.42 | −0.537 | 3.876 |
| ATOM | 15 | H6 | MAN | 1 | 5.012 | 19.624 | 16.571 | +0.10 | +0.37 | +0.424 | 3.876 |
| ATOM | 16 | O3 | MAN | 1 | 3.958 | 24.891 | 17.945 | +0.05 | −0.22 | −0.227 | 3.876 |
| ATOM | 17 | C3 | MAN | 1 | 3.271 | 26.144 | 17.991 | −0.36 | +0.10 | +0.113 | 3.876 |
| ATOM | 18 | C2 | MAN | 1 | 4.273 | 27.235 | 17.628 | −0.37 | +0.11 | +0.113 | 3.876 |
| ATOM | 19 | O2 | MAN | 1 | 5.465 | 27.074 | 18.386 | +0.15 | −0.51 | −0.537 | 3.876 |
| ATOM | 20 | HO2 | MAN | 1 | 5.896 | 27.995 | 18.548 | +0.11 | +0.39 | +0.424 | 3.876 |
| ATOM | 21 | C1 | MAN | 1 | 3.680 | 28.598 | 17.898 | −0.33 | +0.19 | +0.227 | 3.876 |
| ATOM | 22 | O4 | NAG | 1 | 4.628 | 29.570 | 17.644 | +0.02 | −0.20 | −0.227 | 3.876 |
| ATOM | 23 | C4 | NAG | 1 | 4.533 | 30.798 | 18.376 | −0.26 | +0.10 | +0.113 | 3.876 |
| ATOM | 24 | C5 | NAG | 1 | 3.760 | 31.824 | 17.520 | −0.28 | +0.10 | +0.113 | 3.876 |
| ATOM | 25 | C6 | NAG | 1 | 2.258 | 31.594 | 17.485 | −0.50 | +0.13 | +0.113 | 3.876 |
| ATOM | 26 | O6 | NAG | 1 | 1.547 | 32.647 | 18.123 | −0.13 | −0.82 | −0.537 | 3.876 |
| ATOM | 27 | H6 | NAG | 1 | 2.089 | 33.521 | 18.050 | +0.08 | +0.48 | +0.424 | 3.876 |
| ATOM | 28 | O5 | NAG | 1 | 3.938 | 33.134 | 18.041 | +0.06 | −0.19 | −0.227 | 3.876 |
| ATOM | 29 | C1 | NAG | 1 | 5.292 | 33.537 | 18.020 | −0.22 | +0.10 | +0.113 | 3.876 |
| ATOM | 30 | C2 | NAG | 1 | 6.027 | 32.720 | 19.095 | −0.11 | +0.17 | +0.211 | 3.876 |
| ATOM | 31 | N2 | NAG | 1 | 7.414 | 33.113 | 19.205 | −0.03 | −0.51 | −0.650 | 3.876 |
| ATOM | 32 | HN2 | NAG | 1 | 8.087 | 32.825 | 18.482 | +0.11 | +0.39 | +0.440 | 3.876 |
| ATOM | 33 | C7 | NAG | 1 | 7.824 | 33.846 | 20.237 | −0.02 | +0.27 | +0.396 | 3.876 |
| ATOM | 34 | O7 | NAG | 1 | 7.070 | 34.221 | 21.134 | +0.23 | −0.25 | −0.396 | 3.876 |
| ATOM | 35 | C8 | NAG | 1 | 9.293 | 34.227 | 20.282 | −0.01 | +0.00 | +0.000 | 3.876 |
| ATOM | 36 | C3 | NAG | 1 | 5.957 | 31.248 | 18.706 | −0.15 | +0.10 | +0.113 | 3.876 |
| ATOM | 37 | O3 | NAG | 1 | 6.461 | 30.445 | 19.763 | +0.21 | −0.43 | −0.537 | 3.876 |
| ATOM | 38 | HO3 | NAG | 1 | 5.793 | 30.448 | 20.547 | +0.12 | +0.32 | +0.424 | 3.876 |
| ATOM | 39 | O5 | MAN | 1 | 3.264 | 28.683 | 19.270 | +0.09 | −0.18 | −0.227 | 3.876 |
| ATOM | 40 | C5 | MAN | 1 | 2.169 | 27.800 | 19.536 | −0.36 | +0.08 | +0.113 | 3.876 |
| ATOM | 41 | C6 | MAN | 1 | 1.521 | 28.224 | 20.861 | −0.30 | +0.08 | +0.113 | 3.876 |
| ATOM | 42 | O6 | MAN | 1 | 2.427 | 28.061 | 21.982 | +0.13 | −0.18 | −0.227 | 3.876 |
| ATOM | 43 | C1 | MAN | 1 | 2.000 | 28.652 | 23.193 | −0.15 | +0.18 | +0.227 | 3.876 |
| ATOM | 44 | O5 | MAN | 1 | 1.042 | 27.877 | 23.936 | +0.11 | −0.19 | −0.227 | 3.876 |
| ATOM | 45 | C5 | MAN | 1 | 1.570 | 26.707 | 24.611 | −0.40 | +0.11 | +0.113 | 3.876 |
| ATOM | 46 | C6 | MAN | 1 | 0.413 | 25.959 | 25.267 | −0.57 | +0.11 | +0.113 | 3.876 |
| ATOM | 47 | O6 | MAN | 1 | −0.776 | 26.775 | 25.276 | −0.03 | −0.22 | −0.227 | 3.876 |
| ATOM | 48 | C1 | MAN | 1 | −1.424 | 26.787 | 26.523 | −0.54 | +0.26 | +0.227 | 3.876 |
| ATOM | 49 | O5 | MAN | 1 | −0.520 | 26.994 | 27.611 | −0.12 | −0.36 | −0.227 | 3.876 |
| ATOM | 50 | C5 | MAN | 1 | 0.217 | 28.239 | 27.550 | −0.35 | +0.11 | +0.113 | 3.876 |
| ATOM | 51 | C6 | MAN | 1 | 1.108 | 28.294 | 28.774 | −0.48 | +0.09 | +0.113 | 3.876 |
| ATOM | 52 | O6 | MAN | 1 | 2.061 | 27.241 | 28.767 | −0.27 | −0.45 | −0.537 | 3.876 |
| ATOM | 53 | H6 | MAN | 1 | 1.629 | 26.388 | 28.383 | +0.01 | +0.69 | +0.424 | 3.876 |
| ATOM | 54 | C4 | MAN | 1 | −0.738 | 29.437 | 27.500 | −0.21 | +0.10 | +0.113 | 3.876 |
| ATOM | 55 | O4 | MAN | 1 | 0.018 | 30.602 | 27.214 | +0.20 | −0.38 | −0.537 | 3.876 |
| ATOM | 56 | HO4 | MAN | 1 | −0.508 | 31.203 | 26.564 | +0.12 | +0.31 | +0.424 | 3.876 |
| ATOM | 57 | C3 | MAN | 1 | −1.824 | 29.264 | 26.432 | −0.26 | +0.12 | +0.113 | 3.876 |
| ATOM | 58 | O3 | MAN | 1 | −2.808 | 30.277 | 26.585 | +0.13 | −0.52 | −0.537 | 3.876 |
| ATOM | 59 | HO3 | MAN | 1 | −2.983 | 30.726 | 25.674 | +0.10 | +0.44 | +0.424 | 3.876 |
| ATOM | 60 | C2 | MAN | 1 | −2.490 | 27.887 | 26.527 | −0.47 | +0.15 | +0.113 | 3.876 |
| ATOM | 61 | O2 | MAN | 1 | −3.269 | 27.805 | 27.712 | +0.04 | −0.84 | −0.537 | 3.876 |
| ATOM | 62 | HO2 | MAN | 1 | −3.591 | 28.747 | 27.977 | +0.09 | +0.50 | +0.424 | 3.876 |
| ATOM | 63 | C4 | MAN | 1 | 2.673 | 27.115 | 25.593 | −0.36 | +0.11 | +0.113 | 3.876 |
| ATOM | 64 | O4 | MAN | 1 | 3.182 | 25.968 | 26.257 | −0.01 | −0.52 | −0.537 | 3.876 |
| ATOM | 65 | HO4 | MAN | 1 | 2.725 | 25.869 | 27.175 | −0.24 | +0.30 | +0.424 | 3.876 |
| ATOM | 66 | C3 | MAN | 1 | 3.773 | 27.781 | 24.763 | −0.21 | +0.11 | +0.113 | 3.876 |
| ATOM | 67 | O3 | MAN | 1 | 4.964 | 28.082 | 25.542 | +0.16 | −0.16 | −0.227 | 3.876 |
| ATOM | 68 | C1 | BDG | 1 | 6.229 | 27.728 | 24.980 | −0.14 | +0.17 | +0.227 | 3.876 |
| ATOM | 69 | C2 | BDG | 1 | 7.321 | 27.781 | 26.073 | −0.17 | +0.06 | +0.113 | 3.876 |
| ATOM | 70 | O22 | BDG | 1 | 8.543 | 27.273 | 25.531 | +0.19 | −0.07 | −0.113 | 3.876 |
| ATOM | 71 | C3 | BDG | 1 | 7.560 | 29.233 | 26.551 | −0.13 | +0.05 | +0.113 | 3.876 |
| ATOM | 72 | O3 | BDG | 1 | 8.657 | 29.271 | 27.430 | +0.15 | −0.18 | −0.537 | 3.876 |
| ATOM | 73 | HO3 | BDG | 1 | 8.752 | 30.195 | 27.728 | +0.11 | +0.16 | +0.424 | 3.876 |
| ATOM | 74 | C4 | BDG | 1 | 7.801 | 30.211 | 25.373 | −0.03 | +0.06 | +0.113 | 3.876 |
| ATOM | 75 | O4 | BDG | 1 | 7.843 | 31.530 | 25.864 | +0.22 | −0.28 | −0.537 | 3.876 |
| ATOM | 76 | HO4 | BDG | 1 | 8.020 | 32.114 | 25.105 | +0.12 | +0.23 | +0.424 | 3.876 |
| ATOM | 77 | C5 | BDG | 1 | 6.694 | 30.056 | 24.287 | −0.02 | +0.07 | +0.113 | 3.876 |
| ATOM | 78 | C6 | BDG | 1 | 6.973 | 30.868 | 22.990 | +0.00 | +0.07 | +0.113 | 3.876 |
| ATOM | 79 | O6 | BDG | 1 | 6.930 | 32.258 | 23.234 | +0.24 | −0.33 | −0.537 | 3.876 |
| ATOM | 80 | H6 | BDG | 1 | 7.101 | 32.690 | 22.395 | +0.12 | +0.26 | +0.424 | 3.876 |
| ATOM | 81 | O | BDG | 1 | 6.554 | 28.663 | 23.911 | +0.22 | −0.16 | −0.227 | 3.876 |
| ATOM | 82 | C2 | MAN | 1 | 3.210 | 29.032 | 24.073 | −0.09 | +0.09 | +0.113 | 3.876 |
| ATOM | 83 | O2 | MAN | 1 | 2.800 | 29.973 | 25.055 | +0.22 | −0.37 | −0.537 | 3.876 |
| ATOM | 84 | HO2 | MAN | 1 | 2.657 | 30.893 | 24.614 | +0.12 | +0.28 | +0.424 | 3.876 |
| ATOM | 85 | C4 | MAN | 1 | 2.652 | 26.350 | 19.396 | −0.39 | +0.07 | +0.113 | 3.876 |
| ATOM | 86 | O4 | MAN | 1 | 1.555 | 25.464 | 19.565 | +0.33 | −0.12 | −0.537 | 3.876 |
| ATOM | 87 | HO4 | MAN | 1 | 0.705 | 26.006 | 19.778 | −0.08 | +0.12 | +0.424 | 3.876 |
| TER | | | | | | | | | | | |
| ENDMDL | | | | | | | | | | | |
| MODEL | 27 | | | | | | | | | | |
| USER | Run = 27 | | | | | | | | | | |
| USER | Cluster Rank = 2 | | | | | | | | | | |
| USER | Number of conformations in this cluster = 1 | | | | | | | | | | |
| USER | | | | | | | | | | | |
| USER | RMSD from reference structure = 3.931 A | | | | | | | | | | |
| USER | | | | | | | | | | | |
| USER | Estimated Free Energy of Binding = −1.62 kcal/mol [= (1) + (3)] | | | | | | | | | | |
| USER | Estimated Inhibition Constant, Ki = +0.06 [Temperature = 298.15 K] | | | | | | | | | | |
| USER | | | | | | | | | | | |
| USER | Final Docked Energy = −3.49 kcal/mol [= (1) + (2)] | | | | | | | | | | |
| USER | | | | | | | | | | | |
| USER | (1) Final Intermolecular Energy = −6.60 kcal/mol | | | | | | | | | | |
| USER | (2) Final Internal Energy of Ligand = +3.11 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 1.694189 32.372144 19.337776 | | | | | | | | | | |
| USER | NEWDPF quat0 0.714653 −0.681071 −0.159419 −108.396039 | | | | | | | | | | |
| USER | NEWDPF ndihe 16 | | | | | | | | | | |
| USER | NEWDPF dihe0 167.89 69.65 140.96 −36.25 57.89 42.00 −34.82 2.36 36.25 −118.50 | | | | | | | | | | |
| | 140.55 0.05 79.72 −39.12 32.62 −69.59 | | | | | | | | | | |
| USER | | | | | | | | | | | |
| USER | | | | Rank | x | y | z | vdW | Elec | q | RMS |
| ATOM | 1 | C5 | MAN | 2 | 6.438 | 27.291 | 19.756 | −0.07 | +0.10 | +0.113 | 3.931 |
| ATOM | 2 | HO4 | MAN | 2 | 6.774 | 29.102 | 17.813 | +0.10 | +0.41 | +0.424 | 3.931 |
| ATOM | 3 | O4 | MAN | 2 | 7.629 | 28.766 | 18.282 | +0.19 | −0.50 | −0.537 | 3.931 |
| ATOM | 4 | O5 | MAN | 2 | 5.915 | 27.158 | 21.099 | +0.20 | −0.22 | −0.227 | 3.931 |
| ATOM | 5 | HO2 | MAN | 2 | 6.934 | 29.405 | 23.489 | +0.12 | +0.29 | +0.424 | 3.931 |
| ATOM | 6 | O2 | MAN | 2 | 7.222 | 29.205 | 22.521 | +0.23 | −0.38 | −0.537 | 3.931 |
| ATOM | 7 | C2 | MAN | 2 | 6.152 | 29.492 | 21.642 | −0.02 | +0.09 | +0.113 | 3.931 |
| ATOM | 8 | C1 | MAN | 2 | 5.208 | 28.305 | 21.576 | −0.08 | +0.20 | +0.227 | 3.931 |
| ATOM | 9 | C4 | MAN | 2 | 7.353 | 28.522 | 19.650 | −0.04 | +0.09 | +0.113 | 3.931 |
| ATOM | 10 | O3 | MAN | 2 | 7.637 | 30.829 | 20.312 | +0.23 | −0.40 | −0.537 | 3.931 |
| ATOM | 11 | HO3 | MAN | 2 | 7.165 | 31.695 | 20.609 | +0.12 | +0.30 | +0.424 | 3.931 |
| ATOM | 12 | C3 | MAN | 2 | 6.693 | 29.773 | 20.248 | −0.04 | +0.09 | +0.113 | 3.931 |
| ATOM | 13 | C6 | MAN | 2 | 7.171 | 26.001 | 19.433 | −0.09 | +0.11 | +0.113 | 3.931 |
| ATOM | 14 | O6 | MAN | 2 | 6.659 | 25.393 | 18.253 | +0.17 | −0.55 | −0.537 | 3.931 |
| ATOM | 15 | H6 | MAN | 2 | 6.192 | 24.509 | 18.498 | +0.11 | +0.44 | +0.424 | 3.931 |
| ATOM | 16 | O3 | MAN | 2 | 4.143 | 28.643 | 20.745 | +0.19 | −0.19 | −0.227 | 3.931 |
| ATOM | 17 | C3 | MAN | 2 | 3.041 | 27.732 | 20.758 | −0.23 | +0.09 | +0.113 | 3.931 |
| ATOM | 18 | C2 | MAN | 2 | 3.140 | 26.867 | 19.506 | −0.33 | +0.09 | +0.113 | 3.931 |
| ATOM | 19 | O2 | MAN | 2 | 3.342 | 27.688 | 18.363 | +0.04 | −0.43 | −0.537 | 3.931 |
| ATOM | 20 | HO2 | MAN | 2 | 2.917 | 27.239 | 17.539 | +0.08 | +0.35 | +0.424 | 3.931 |
| ATOM | 21 | C1 | MAN | 2 | 1.877 | 26.056 | 19.332 | −0.36 | +0.11 | +0.227 | 3.931 |
| ATOM | 22 | O4 | NAG | 2 | 1.948 | 25.344 | 18.151 | +0.05 | −0.18 | −0.227 | 3.931 |
| ATOM | 23 | C4 | NAG | 2 | 1.940 | 23.913 | 18.238 | −0.58 | +0.06 | +0.113 | 3.931 |
| ATOM | 24 | C5 | NAG | 2 | 3.353 | 23.435 | 18.630 | −0.38 | +0.09 | +0.113 | 3.931 |
| ATOM | 25 | C6 | NAG | 2 | 3.691 | 23.637 | 20.098 | −0.32 | +0.12 | +0.113 | 3.931 |
| ATOM | 26 | O6 | NAG | 2 | 3.850 | 22.400 | 20.780 | −0.13 | −0.70 | −0.537 | 3.931 |
| ATOM | 27 | H6 | NAG | 2 | 4.172 | 21.681 | 20.116 | +0.09 | +0.46 | +0.424 | 3.931 |
| ATOM | 28 | O5 | NAG | 2 | 3.485 | 22.040 | 18.393 | +0.07 | −0.16 | −0.227 | 3.931 |
| ATOM | 29 | C1 | NAG | 2 | 3.318 | 21.712 | 17.029 | −0.35 | +0.08 | +0.113 | 3.931 |
| ATOM | 30 | C2 | NAG | 2 | 1.829 | 21.901 | 16.695 | −0.40 | +0.08 | +0.211 | 3.931 |
| ATOM | 31 | N2 | NAG | 2 | 1.537 | 21.518 | 15.332 | −0.31 | −0.48 | −0.650 | 3.931 |
| ATOM | 32 | HN2 | NAG | 2 | 2.176 | 21.794 | 14.574 | +0.08 | +0.44 | +0.440 | 3.931 |
| ATOM | 33 | C7 | NAG | 2 | 0.446 | 20.808 | 15.052 | −0.60 | +0.29 | +0.396 | 3.931 |
| ATOM | 34 | O7 | NAG | 2 | −0.356 | 20.447 | 15.912 | +0.02 | −0.13 | −0.396 | 3.931 |
| ATOM | 35 | C8 | NAG | 2 | 0.210 | 20.437 | 13.599 | −0.44 | +0.00 | +0.000 | 3.931 |
| ATOM | 36 | C3 | NAG | 2 | 1.480 | 23.373 | 16.883 | −0.61 | +0.07 | +0.113 | 3.931 |
| ATOM | 37 | O3 | NAG | 2 | 0.077 | 23.555 | 16.765 | −0.19 | −0.33 | −0.537 | 3.931 |
| ATOM | 38 | HO3 | NAG | 2 | −0.392 | 23.160 | 17.592 | −0.29 | −0.13 | +0.424 | 3.931 |
| ATOM | 39 | O5 | MAN | 2 | 0.740 | 26.932 | 19.295 | +0.24 | −0.12 | −0.227 | 3.931 |
| ATOM | 40 | C5 | MAN | 2 | 0.536 | 27.573 | 20.559 | −0.43 | +0.06 | +0.113 | 3.931 |
| ATOM | 41 | C6 | MAN | 2 | −0.884 | 28.153 | 20.580 | −0.46 | +0.09 | +0.113 | 3.931 |
| ATOM | 42 | O6 | MAN | 2 | −0.955 | 29.436 | 19.906 | +0.03 | −0.20 | −0.227 | 3.931 |
| ATOM | 43 | C1 | MAN | 2 | −1.789 | 30.395 | 20.524 | −0.31 | +0.24 | +0.227 | 3.931 |
| ATOM | 44 | O5 | MAN | 2 | −2.159 | 30.089 | 21.880 | +0.04 | −0.32 | −0.227 | 3.931 |
| ATOM | 45 | C5 | MAN | 2 | −1.111 | 30.259 | 22.869 | −0.16 | +0.12 | +0.113 | 3.931 |
| ATOM | 46 | C6 | MAN | 2 | −1.644 | 29.806 | 24.225 | −0.19 | +0.14 | +0.113 | 3.931 |
| ATOM | 47 | O6 | MAN | 2 | −0.622 | 29.106 | 24.962 | +0.16 | −0.21 | −0.227 | 3.931 |
| ATOM | 48 | C1 | MAN | 2 | −1.019 | 28.771 | 26.268 | −0.25 | +0.22 | +0.227 | 3.931 |
| ATOM | 49 | O5 | MAN | 2 | −1.367 | 27.390 | 26.401 | −0.02 | −0.26 | −0.227 | 3.931 |
| ATOM | 50 | C5 | MAN | 2 | −0.300 | 26.468 | 26.076 | −0.58 | +0.12 | +0.113 | 3.931 |
| ATOM | 51 | C6 | MAN | 2 | −0.839 | 25.067 | 26.279 | −0.68 | +0.11 | +0.113 | 3.931 |
| ATOM | 52 | O6 | MAN | 2 | −2.251 | 25.030 | 26.130 | −0.31 | −0.33 | −0.537 | 3.931 |
| ATOM | 53 | H6 | MAN | 2 | −2.661 | 25.888 | 26.525 | −0.19 | +0.35 | +0.424 | 3.931 |
| ATOM | 54 | C4 | MAN | 2 | 0.931 | 26.726 | 26.952 | −0.41 | +0.14 | +0.113 | 3.931 |
| ATOM | 55 | O4 | MAN | 2 | 2.011 | 25.960 | 26.447 | −0.09 | −0.58 | −0.537 | 3.931 |
| ATOM | 56 | HO4 | MAN | 2 | 2.879 | 26.513 | 26.498 | +0.04 | +0.36 | +0.424 | 3.931 |
| ATOM | 57 | C3 | MAN | 2 | 1.325 | 28.208 | 26.970 | −0.29 | +0.10 | +0.113 | 3.931 |
| ATOM | 58 | O3 | MAN | 2 | 2.311 | 28.426 | 27.969 | +0.05 | −0.35 | −0.537 | 3.931 |
| ATOM | 59 | HO3 | MAN | 2 | 3.099 | 28.954 | 27.565 | +0.10 | +0.24 | +0.424 | 3.931 |
| ATOM | 60 | C2 | MAN | 2 | 0.115 | 29.108 | 27.242 | −0.22 | +0.09 | +0.113 | 3.931 |
| ATOM | 61 | O2 | MAN | 2 | −0.327 | 28.936 | 28.580 | +0.05 | −0.47 | −0.537 | 3.931 |
| ATOM | 62 | HO2 | MAN | 2 | 0.468 | 28.652 | 29.171 | +0.08 | +0.32 | +0.424 | 3.931 |
| ATOM | 63 | C4 | MAN | 2 | −0.605 | 31.705 | 22.860 | −0.07 | +0.10 | +0.113 | 3.931 |
| ATOM | 64 | O4 | MAN | 2 | 0.416 | 31.869 | 23.834 | +0.23 | −0.39 | −0.537 | 3.931 |
| ATOM | 65 | HO4 | MAN | 2 | 0.010 | 32.264 | 24.695 | +0.12 | +0.30 | +0.424 | 3.931 |
| ATOM | 66 | C3 | MAN | 2 | −0.047 | 31.964 | 21.458 | −0.10 | +0.10 | +0.113 | 3.931 |
| ATOM | 67 | O3 | MAN | 2 | 0.617 | 33.253 | 21.344 | +0.20 | −0.17 | −0.227 | 3.931 |
| ATOM | 68 | C1 | BDG | 2 | 2.041 | 33.255 | 21.228 | −0.07 | +0.16 | +0.227 | 3.931 |
| ATOM | 69 | C2 | BDG | 2 | 2.681 | 33.365 | 22.631 | −0.01 | +0.07 | +0.113 | 3.931 |
| ATOM | 70 | O22 | BDG | 2 | 4.097 | 33.206 | 22.507 | +0.23 | −0.07 | −0.113 | 3.931 |
| ATOM | 71 | C3 | BDG | 2 | 2.394 | 34.746 | 23.267 | −0.01 | +0.06 | +0.113 | 3.931 |
| ATOM | 72 | O3 | BDG | 2 | 3.110 | 34.875 | 24.470 | +0.24 | −0.30 | −0.537 | 3.931 |
| ATOM | 73 | HO3 | BDG | 2 | 2.877 | 35.743 | 24.849 | +0.12 | +0.22 | +0.424 | 3.931 |
| ATOM | 74 | C4 | BDG | 2 | 2.751 | 35.920 | 22.318 | −0.02 | +0.06 | +0.113 | 3.931 |
| ATOM | 75 | O4 | BDG | 2 | 2.311 | 37.129 | 22.889 | +0.23 | −0.27 | −0.537 | 3.931 |
| ATOM | 76 | HO4 | BDG | 2 | 2.573 | 37.845 | 22.282 | +0.12 | +0.20 | +0.424 | 3.931 |
| ATOM | 77 | C5 | BDG | 2 | 2.120 | 35.706 | 20.910 | −0.10 | +0.06 | +0.113 | 3.931 |
| ATOM | 78 | C6 | BDG | 2 | 2.591 | 36.745 | 19.851 | −0.17 | +0.05 | +0.113 | 3.931 |
| ATOM | 79 | O6 | BDG | 2 | 2.135 | 38.043 | 20.166 | +0.16 | −0.20 | −0.537 | 3.931 |
| ATOM | 80 | H6 | BDG | 2 | 2.453 | 38.622 | 19.471 | +0.10 | +0.16 | +0.424 | 3.931 |
| ATOM | 81 | O | BDG | 2 | 2.451 | 34.387 | 20.407 | +0.18 | −0.15 | −0.227 | 3.931 |
| ATOM | 82 | C2 | MAN | 2 | −1.169 | 31.806 | 20.421 | −0.22 | +0.12 | +0.113 | 3.931 |
| ATOM | 83 | O2 | MAN | 2 | −2.176 | 32.780 | 20.658 | +0.16 | −0.52 | −0.537 | 3.931 |
| ATOM | 84 | HO2 | MAN | 2 | −2.795 | 32.843 | 19.837 | +0.10 | +0.44 | +0.424 | 3.931 |
| ATOM | 85 | C4 | MAN | 2 | 1.711 | 28.521 | 20.832 | −0.26 | +0.08 | +0.113 | 3.931 |
| ATOM | 86 | O4 | MAN | 2 | 1.570 | 29.094 | 22.124 | +0.17 | −0.41 | −0.537 | 3.931 |
| ATOM | 87 | HO4 | MAN | 2 | 0.694 | 28.763 | 22.553 | +0.11 | +0.33 | +0.424 | 3.931 |
| TER | | | | | | | | | | | |
| ENDMDL | | | | | | | | | | | |
| MODEL | 44 | | | | | | | | | | |
| USER | Run = 44 | | | | | | | | | | |
| USER | Cluster Rank = 3 | | | | | | | | | | |
| USER | Number of conformations in this cluster = 1 | | | | | | | | | | |
| USER | | | | | | | | | | | |
| USER | RMSD from reference structure = 3.305 A | | | | | | | | | | |
| USER | | | | | | | | | | | |
| USER | Estimated Free Energy of Binding = −1.02 kcal/mol [= (1) + (3)] | | | | | | | | | | |
| USER | Estimated Inhibition Constant, Ki = +0.18 [Temperature = 298.15 K] | | | | | | | | | | |
| USER | | | | | | | | | | | |
| USER | Final Docked Energy = −3.31 kcal/mol [= (1) + (2)] | | | | | | | | | | |
| USER | | | | | | | | | | | |
| USER | (1) Final Intermolecular Energy = −6.00 kcal/mol | | | | | | | | | | |
| USER | (2) Final Internal Energy of Ligand = +2.69 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran 0 0.307770 24.956877 25.547332 | | | | | | | | | | |
| USER | NEWDPF quat 0 −0.005335 0.366988 −0.930211 −175.317889 | | | | | | | | | | |
| USER | NEWDPF ndihe 16 | | | | | | | | | | |
| USER | NEWDPF dihe 0 21.44 9.17 89.87 −13.53 −29.04 110.30 −8.62 −48.09 −20.19 −52.05 | | | | | | | | | | |
| | 81.95 75.07 136.16 180.00 63.07 −43.38 | | | | | | | | | | |
| USER | | | | | | | | | | | |
| USER | | | | Rank | x | y | z | vdW | Elec | q | RMS |
| ATOM | 1 | C5 | MAN | 3 | −3.045 | 30.539 | 28.016 | −0.17 | +0.10 | +0.113 | 3.305 |
| ATOM | 2 | HO4 | MAN | 3 | −3.972 | 28.142 | 28.767 | +0.04 | +0.54 | +0.424 | 3.305 |
| ATOM | 3 | O4 | MAN | 3 | −3.986 | 28.884 | 29.481 | +0.01 | −0.55 | −0.537 | 3.305 |
| ATOM | 4 | O5 | MAN | 3 | −1.817 | 31.213 | 27.657 | +0.20 | −0.17 | −0.227 | 3.305 |
| ATOM | 5 | HO2 | MAN | 3 | 0.893 | 30.916 | 29.747 | +0.09 | +0.18 | +0.424 | 3.305 |
| ATOM | 6 | O2 | MAN | 3 | −0.098 | 30.646 | 29.812 | +0.08 | −0.26 | −0.537 | 3.305 |
| ATOM | 7 | C2 | MAN | 3 | −0.387 | 29.671 | 28.829 | −0.30 | +0.08 | +0.113 | 3.305 |
| ATOM | 8 | C1 | MAN | 3 | −0.688 | 30.349 | 27.505 | −0.12 | +0.17 | +0.227 | 3.305 |
| ATOM | 9 | C4 | MAN | 3 | −2.860 | 29.727 | 29.309 | −0.30 | +0.10 | +0.113 | 3.305 |
| ATOM | 10 | O3 | MAN | 3 | −1.357 | 28.282 | 30.530 | −0.08 | −0.51 | −0.537 | 3.305 |
| ATOM | 11 | HO3 | MAN | 3 | −0.577 | 27.612 | 30.464 | +0.00 | +0.45 | +0.424 | 3.305 |
| ATOM | 12 | C3 | MAN | 3 | −1.598 | 28.854 | 29.256 | −0.44 | +0.12 | +0.113 | 3.305 |
| ATOM | 13 | C6 | MAN | 3 | −4.116 | 31.607 | 28.157 | −0.10 | +0.09 | +0.113 | 3.305 |
| ATOM | 14 | O6 | MAN | 3 | −4.351 | 32.274 | 26.923 | +0.19 | −0.43 | −0.537 | 3.305 |
| ATOM | 15 | H6 | MAN | 3 | −5.204 | 31.899 | 26.487 | +0.11 | +0.38 | +0.424 | 3.305 |
| ATOM | 16 | O3 | MAN | 3 | −0.915 | 29.359 | 26.553 | +0.16 | −0.21 | −0.227 | 3.305 |
| ATOM | 17 | C3 | MAN | 3 | −0.420 | 29.644 | 25.242 | −0.13 | +0.10 | +0.113 | 3.305 |
| ATOM | 18 | C2 | MAN | 3 | −1.297 | 30.738 | 24.643 | −0.12 | +0.11 | +0.113 | 3.305 |
| ATOM | 19 | O2 | MAN | 3 | −2.669 | 30.405 | 24.804 | +0.11 | −0.67 | −0.537 | 3.305 |
| ATOM | 20 | HO2 | MAN | 3 | −3.213 | 30.802 | 24.024 | +0.10 | +0.62 | +0.424 | 3.305 |
| ATOM | 21 | C1 | MAN | 3 | −0.985 | 30.910 | 23.175 | −0.12 | +0.22 | +0.227 | 3.305 |
| ATOM | 22 | O4 | NAG | 3 | −1.844 | 31.845 | 22.630 | +0.18 | −0.23 | −0.227 | 3.305 |
| ATOM | 23 | C4 | NAG | 3 | −1.752 | 32.089 | 21.221 | −0.15 | +0.11 | +0.113 | 3.305 |
| ATOM | 24 | C5 | NAG | 3 | −1.646 | 33.612 | 20.990 | −0.14 | +0.09 | +0.113 | 3.305 |
| ATOM | 25 | C6 | NAG | 3 | −0.275 | 34.191 | 21.298 | −0.11 | +0.07 | +0.113 | 3.305 |
| ATOM | 26 | O6 | NAG | 3 | 0.367 | 34.682 | 20.128 | +0.09 | −0.30 | −0.537 | 3.305 |
| ATOM | 27 | H6 | NAG | 3 | −0.345 | 34.982 | 19.446 | −0.18 | +0.07 | +0.424 | 3.305 |
| ATOM | 28 | O5 | NAG | 3 | −1.899 | 33.923 | 19.627 | +0.06 | −0.19 | −0.227 | 3.305 |
| ATOM | 29 | C1 | NAG | 3 | −3.202 | 33.549 | 19.227 | −0.25 | +0.11 | +0.113 | 3.305 |
| ATOM | 30 | C2 | NAG | 3 | −3.234 | 32.013 | 19.162 | −0.31 | +0.25 | +0.211 | 3.305 |
| ATOM | 31 | N2 | NAG | 3 | −4.511 | 31.529 | 18.688 | −0.19 | −0.74 | −0.650 | 3.305 |
| ATOM | 32 | HN2 | NAG | 3 | −5.341 | 32.136 | 18.738 | +0.11 | +0.47 | +0.440 | 3.305 |
| ATOM | 33 | C7 | NAG | 3 | −4.620 | 30.301 | 18.185 | −0.53 | +0.43 | +0.396 | 3.305 |
| ATOM | 34 | O7 | NAG | 3 | −3.667 | 29.527 | 18.098 | −0.09 | −0.43 | −0.396 | 3.305 |
| ATOM | 35 | C8 | NAG | 3 | −5.990 | 29.864 | 17.700 | −0.61 | +0.00 | +0.000 | 3.305 |
| ATOM | 36 | C3 | NAG | 3 | −2.987 | 31.470 | 20.564 | −0.23 | +0.13 | +0.113 | 3.305 |
| ATOM | 37 | O3 | NAG | 3 | −2.825 | 30.061 | 20.514 | +0.06 | −0.66 | −0.537 | 3.305 |
| ATOM | 38 | HO3 | NAG | 3 | −1.920 | 29.833 | 20.079 | +0.09 | +0.44 | +0.424 | 3.305 |
| ATOM | 39 | O5 | MAN | 3 | −1.147 | 29.657 | 22.494 | +0.06 | −0.25 | −0.227 | 3.305 |
| ATOM | 40 | C5 | MAN | 3 | −0.166 | 28.705 | 22.919 | −0.24 | +0.10 | +0.113 | 3.305 |
| ATOM | 41 | C6 | MAN | 3 | −0.129 | 27.563 | 21.894 | −0.37 | +0.07 | +0.113 | 3.305 |
| ATOM | 42 | O6 | MAN | 3 | 1.230 | 27.160 | 21.584 | +0.12 | −0.10 | −0.227 | 3.305 |
| ATOM | 43 | C1 | MAN | 3 | 1.875 | 27.930 | 20.590 | −0.32 | +0.15 | +0.227 | 3.305 |
| ATOM | 44 | O5 | MAN | 3 | 2.609 | 29.063 | 21.087 | +0.17 | −0.17 | −0.227 | 3.305 |
| ATOM | 45 | C5 | MAN | 3 | 3.857 | 28.755 | 21.759 | −0.10 | +0.09 | +0.113 | 3.305 |
| ATOM | 46 | C6 | MAN | 3 | 4.452 | 30.053 | 22.298 | −0.03 | +0.08 | +0.113 | 3.305 |
| ATOM | 47 | O6 | MAN | 3 | 3.852 | 30.399 | 23.562 | +0.23 | −0.16 | −0.227 | 3.305 |
| ATOM | 48 | C1 | MAN | 3 | 4.777 | 30.434 | 24.618 | −0.03 | +0.14 | +0.227 | 3.305 |
| ATOM | 49 | O5 | MAN | 3 | 4.218 | 30.963 | 25.823 | +0.22 | −0.13 | −0.227 | 3.305 |
| ATOM | 50 | C5 | MAN | 3 | 3.083 | 30.224 | 26.333 | −0.10 | +0.07 | +0.113 | 3.305 |
| ATOM | 51 | C6 | MAN | 3 | 2.631 | 30.913 | 27.604 | −0.15 | +0.06 | +0.113 | 3.305 |
| ATOM | 52 | O6 | MAN | 3 | 3.697 | 31.631 | 28.210 | +0.15 | −0.25 | −0.537 | 3.305 |
| ATOM | 53 | H6 | MAN | 3 | 4.574 | 31.101 | 28.099 | +0.09 | +0.19 | +0.424 | 3.305 |
| ATOM | 54 | C4 | MAN | 3 | 3.455 | 28.760 | 26.594 | −0.21 | +0.07 | +0.113 | 3.305 |
| ATOM | 55 | O4 | MAN | 3 | 2.268 | 28.036 | 26.871 | +0.10 | −0.41 | −0.537 | 3.305 |
| ATOM | 56 | HO4 | MAN | 3 | 2.317 | 27.111 | 26.421 | +0.09 | +0.37 | +0.424 | 3.305 |
| ATOM | 57 | C3 | MAN | 3 | 4.169 | 28.125 | 25.395 | −0.18 | +0.09 | +0.113 | 3.305 |
| ATOM | 58 | O3 | MAN | 3 | 4.696 | 26.860 | 25.769 | +0.01 | −0.48 | −0.537 | 3.305 |
| ATOM | 59 | HO3 | MAN | 3 | 4.417 | 26.152 | 25.074 | +0.08 | +0.68 | +0.424 | 3.305 |
| ATOM | 60 | C2 | MAN | 3 | 5.302 | 29.017 | 24.877 | −0.08 | +0.08 | +0.113 | 3.305 |
| ATOM | 61 | O2 | MAN | 3 | 6.360 | 29.056 | 25.823 | +0.19 | −0.30 | −0.537 | 3.305 |
| ATOM | 62 | HO2 | MAN | 3 | 6.365 | 28.182 | 26.370 | +0.11 | +0.21 | +0.424 | 3.305 |
| ATOM | 63 | C4 | MAN | 3 | 4.794 | 27.996 | 20.814 | −0.11 | +0.10 | +0.113 | 3.305 |
| ATOM | 64 | O4 | MAN | 3 | 6.017 | 27.703 | 21.474 | +0.21 | −0.48 | −0.537 | 3.305 |
| ATOM | 65 | HO4 | MAN | 3 | 6.722 | 28.412 | 21.225 | +0.12 | +0.34 | +0.424 | 3.305 |
| ATOM | 66 | C3 | MAN | 3 | 4.074 | 26.701 | 20.432 | −0.22 | +0.11 | +0.113 | 3.305 |
| ATOM | 67 | O3 | MAN | 3 | 4.910 | 25.796 | 19.659 | +0.14 | −0.23 | −0.227 | 3.305 |
| ATOM | 68 | C1 | BDG | 3 | 4.909 | 24.416 | 20.027 | −0.24 | +0.28 | +0.227 | 3.305 |
| ATOM | 69 | C2 | BDG | 3 | 5.880 | 23.629 | 19.117 | −0.22 | +0.12 | +0.113 | 3.305 |
| ATOM | 70 | O22 | BDG | 3 | 5.984 | 22.288 | 19.601 | +0.07 | −0.12 | −0.113 | 3.305 |
| ATOM | 71 | C3 | BDG | 3 | 5.365 | 23.588 | 17.658 | −0.29 | +0.12 | +0.113 | 3.305 |
| ATOM | 72 | O3 | BDG | 3 | 6.183 | 22.744 | 16.888 | +0.04 | −0.58 | −0.537 | 3.305 |
| ATOM | 73 | HO3 | BDG | 3 | 5.842 | 22.772 | 15.974 | −0.22 | +0.48 | +0.424 | 3.305 |
| ATOM | 74 | C4 | BDG | 3 | 3.891 | 23.115 | 17.565 | −0.35 | +0.10 | +0.113 | 3.305 |
| ATOM | 75 | O4 | BDG | 3 | 3.439 | 23.262 | 16.240 | +0.05 | −0.63 | −0.537 | 3.305 |
| ATOM | 76 | HO4 | BDG | 3 | 2.522 | 22.933 | 16.211 | +0.08 | +0.39 | +0.424 | 3.305 |
| ATOM | 77 | C5 | BDG | 3 | 2.988 | 23.912 | 18.553 | −0.43 | +0.08 | +0.113 | 3.305 |
| ATOM | 78 | C6 | BDG | 3 | 1.535 | 23.365 | 18.653 | −0.58 | +0.03 | +0.113 | 3.305 |
| ATOM | 79 | O6 | BDG | 3 | 0.837 | 23.536 | 17.438 | −0.17 | −0.23 | −0.537 | 3.305 |
| ATOM | 80 | H6 | BDG | 3 | −0.045 | 23.187 | 17.577 | −0.28 | −0.07 | +0.424 | 3.305 |
| ATOM | 81 | O | BDG | 3 | 3.559 | 23.886 | 19.886 | −0.02 | −0.21 | −0.227 | 3.305 |
| ATOM | 82 | C2 | MAN | 3 | 2.766 | 27.037 | 19.699 | −0.35 | +0.08 | +0.113 | 3.305 |
| ATOM | 83 | O2 | MAN | 3 | 3.062 | 27.724 | 18.491 | +0.03 | −0.42 | −0.537 | 3.305 |
| ATOM | 84 | HO2 | MAN | 3 | 2.228 | 27.732 | 17.886 | +0.15 | +0.27 | +0.424 | 3.305 |
| ATOM | 85 | C4 | MAN | 3 | −0.420 | 28.350 | 24.391 | −0.26 | +0.11 | +0.113 | 3.305 |
| ATOM | 86 | O4 | MAN | 3 | 0.601 | 27.478 | 24.853 | +0.08 | −0.47 | −0.537 | 3.305 |
| ATOM | 87 | HO4 | MAN | 3 | 1.246 | 27.263 | 24.079 | +0.09 | +0.37 | +0.424 | 3.305 |
| TER | | | | | | | | | | | |
| ENDMDL | | | | | | | | | | | |
| MODEL | 87 | | | | | | | | | | |
| USER | Run = 87 | | | | | | | | | | |
| USER | Cluster Rank = 4 | | | | | | | | | | |
| USER | Number of conformations in this cluster = 1 | | | | | | | | | | |
| USER | | | | | | | | | | | |
| USER | RMSD from reference structure = 4.101 A | | | | | | | | | | |
| USER | | | | | | | | | | | |
| USER | Estimated Free Energy of Binding = −3.61 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −3.10 kcal/mol [= (1) + (2)] | | | | | | | | | | |
| USER | | | | | | | | | | | |
| USER | (1) Final Intermolecular Energy = −8.59 kcal/mol | | | | | | | | | | |
| USER | (2) Final Internal Energy of Ligand = +5.49 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran 0 3.844980 31.727590 24.726726 | | | | | | | | | | |
| USER | NEWDPF quat 0 0.766579 −0.628720 −0.130648 −47.615333 | | | | | | | | | | |
| USER | NEWDPF ndihe 16 | | | | | | | | | | |
| USER | NEWDPF dihe 0 −64.60 −42.96 −18.43 −41.54 −4.93 74.07 161.30 −85.42 | | | | | | | | | | |
| | 125.74 179.85 39.40 17.85 −4.84 102.66 14.76 −54.09 | | | | | | | | | | |
| USER | | | | | | | | | | | |
| USER | | | | Rank | x | y | z | vdW | Elec | q | RMS |
| ATOM | 1 | C5 | MAN | 4 | 7.724 | 25.951 | 24.456 | −0.21 | +0.08 | +0.113 | 4.101 |
| ATOM | 2 | HO4 | MAN | 4 | 9.019 | 28.259 | 24.859 | +0.12 | +0.25 | +0.424 | 4.101 |
| ATOM | 3 | O4 | MAN | 4 | 9.369 | 27.438 | 25.376 | +0.20 | −0.32 | −0.537 | 4.101 |
| ATOM | 4 | O5 | MAN | 4 | 6.458 | 25.304 | 24.725 | −0.03 | −0.23 | −0.227 | 4.101 |
| ATOM | 5 | HO2 | MAN | 4 | 5.259 | 25.314 | 27.944 | −0.12 | +0.03 | +0.424 | 4.101 |
| ATOM | 6 | O2 | MAN | 4 | 6.149 | 25.590 | 27.508 | −0.29 | −0.32 | −0.537 | 4.101 |
| ATOM | 7 | C2 | MAN | 4 | 5.928 | 26.676 | 26.630 | −0.39 | +0.06 | +0.113 | 4.101 |
| ATOM | 8 | C1 | MAN | 4 | 5.459 | 26.161 | 25.281 | −0.32 | +0.25 | +0.227 | 4.101 |
| ATOM | 9 | C4 | MAN | 4 | 8.281 | 26.604 | 25.732 | −0.20 | +0.07 | +0.113 | 4.101 |
| ATOM | 10 | O3 | MAN | 4 | 7.709 | 27.874 | 27.706 | +0.13 | −0.04 | −0.537 | 4.101 |
| ATOM | 11 | HO3 | MAN | 4 | 7.042 | 28.535 | 28.131 | −0.19 | −0.08 | +0.424 | 4.101 |
| ATOM | 12 | C3 | MAN | 4 | 7.221 | 27.457 | 26.442 | −0.23 | +0.06 | +0.113 | 4.101 |
| ATOM | 13 | C6 | MAN | 4 | 8.658 | 24.890 | 23.899 | −0.24 | +0.08 | +0.113 | 4.101 |
| ATOM | 14 | O6 | MAN | 4 | 7.957 | 23.695 | 23.580 | +0.03 | −0.34 | −0.537 | 4.101 |
| ATOM | 15 | H6 | MAN | 4 | 6.946 | 23.885 | 23.559 | +0.03 | +0.39 | +0.424 | 4.101 |
| ATOM | 16 | O3 | MAN | 4 | 5.202 | 27.258 | 24.464 | +0.04 | −0.24 | −0.227 | 4.101 |
| ATOM | 17 | C3 | MAN | 4 | 4.243 | 27.038 | 23.427 | +0.27 | +0.17 | +0.113 | 4.101 |
| ATOM | 18 | C2 | MAN | 4 | 4.941 | 27.278 | 22.092 | −0.14 | +0.13 | +0.113 | 4.101 |
| ATOM | 19 | O2 | MAN | 4 | 5.648 | 28.511 | 22.127 | +0.22 | −0.45 | −0.537 | 4.101 |
| ATOM | 20 | HO2 | MAN | 4 | 5.674 | 28.919 | 21.181 | +0.12 | +0.34 | +0.424 | 4.101 |
| ATOM | 21 | C1 | MAN | 4 | 3.927 | 27.306 | 20.972 | −0.19 | +0.21 | +0.227 | 4.101 |
| ATOM | 22 | O4 | NAG | 4 | 4.565 | 27.607 | 19.784 | +0.17 | −0.20 | −0.227 | 4.101 |
| ATOM | 23 | C4 | NAG | 4 | 5.663 | 26.774 | 19.390 | −0.13 | +0.11 | +0.113 | 4.101 |
| ATOM | 24 | C5 | NAG | 4 | 6.420 | 27.469 | 18.239 | −0.15 | +0.11 | +0.113 | 4.101 |
| ATOM | 25 | C6 | NAG | 4 | 7.303 | 28.623 | 18.685 | −0.09 | +0.10 | +0.113 | 4.101 |
| ATOM | 26 | O6 | NAG | 4 | 8.682 | 28.342 | 18.481 | +0.21 | −0.48 | −0.537 | 4.101 |
| ATOM | 27 | H6 | NAG | 4 | 8.787 | 27.692 | 17.689 | +0.11 | +0.41 | +0.424 | 4.101 |
| ATOM | 28 | O5 | NAG | 4 | 7.286 | 26.548 | 17.592 | +0.17 | −0.23 | −0.227 | 4.101 |
| ATOM | 29 | C1 | NAG | 4 | 6.582 | 25.469 | 17.012 | −0.29 | +0.14 | +0.113 | 4.101 |
| ATOM | 30 | C2 | NAG | 4 | 6.067 | 24.590 | 18.163 | −0.21 | +0.22 | +0.211 | 4.101 |
| ATOM | 31 | N2 | NAG | 4 | 5.391 | 23.411 | 17.671 | −0.17 | −0.70 | −0.650 | 4.101 |
| ATOM | 32 | HN2 | NAG | 4 | 4.679 | 23.499 | 16.932 | +0.08 | +0.53 | +0.440 | 4.101 |
| ATOM | 33 | C7 | NAG | 4 | 5.684 | 22.211 | 18.167 | −0.29 | +0.38 | +0.396 | 4.101 |
| ATOM | 34 | O7 | NAG | 4 | 6.524 | 22.039 | 19.049 | +0.05 | −0.39 | −0.396 | 4.101 |
| ATOM | 35 | C8 | NAG | 4 | 4.939 | 21.019 | 17.595 | −0.29 | +0.00 | +0.000 | 4.101 |
| ATOM | 36 | C3 | NAG | 4 | 5.089 | 25.413 | 18.993 | −0.20 | +0.11 | +0.113 | 4.101 |
| ATOM | 37 | O3 | NAG | 4 | 4.735 | 24.701 | 20.169 | −0.02 | −0.68 | −0.537 | 4.101 |
| ATOM | 38 | HO3 | NAG | 4 | 5.536 | 24.683 | 20.815 | +0.10 | +0.62 | +0.424 | 4.101 |
| ATOM | 39 | O5 | MAN | 4 | 2.929 | 28.301 | 21.245 | +0.15 | −0.18 | −0.227 | 4.101 |
| ATOM | 40 | C5 | MAN | 4 | 2.138 | 27.943 | 22.383 | −0.23 | +0.09 | +0.113 | 4.101 |
| ATOM | 41 | C6 | MAN | 4 | 0.868 | 28.805 | 22.374 | −0.19 | +0.09 | +0.113 | 4.101 |
| ATOM | 42 | C6 | MAN | 4 | 0.084 | 28.601 | 21.170 | +0.06 | −0.17 | −0.227 | 4.101 |
| ATOM | 43 | C1 | MAN | 4 | −1.305 | 28.441 | 21.375 | −0.43 | +0.21 | +0.227 | 4.101 |
| ATOM | 44 | O5 | MAN | 4 | −1.842 | 29.191 | 22.479 | −0.18 | −0.34 | −0.227 | 4.101 |
| ATOM | 45 | C5 | MAN | 4 | −1.966 | 30.620 | 22.264 | −0.10 | +0.14 | +0.113 | 4.101 |
| ATOM | 46 | C6 | MAN | 4 | −2.460 | 31.265 | 23.556 | −0.01 | +0.14 | +0.113 | 4.101 |
| ATOM | 47 | O6 | MAN | 4 | −1.821 | 30.663 | 24.699 | +0.14 | −0.24 | −0.227 | 4.101 |
| ATOM | 48 | C1 | MAN | 4 | −2.593 | 29.647 | 25.287 | −0.19 | +0.29 | +0.227 | 4.101 |
| ATOM | 49 | O5 | MAN | 4 | −1.824 | 28.484 | 25.603 | +0.02 | −0.27 | −0.227 | 4.101 |
| ATOM | 50 | C5 | MAN | 4 | −0.733 | 28.718 | 26.524 | −0.25 | +0.11 | +0.113 | 4.101 |
| ATOM | 51 | C6 | MAN | 4 | −0.041 | 27.389 | 26.750 | −0.42 | +0.13 | +0.113 | 4.101 |
| ATOM | 52 | O6 | MAN | 4 | 0.201 | 27.160 | 28.130 | −0.19 | −0.94 | −0.537 | 4.101 |
| ATOM | 53 | H6 | MAN | 4 | 0.455 | 28.048 | 28.587 | +0.08 | +0.44 | +0.424 | 4.101 |
| ATOM | 54 | C4 | MAN | 4 | −1.246 | 29.311 | 27.841 | −0.26 | +0.10 | +0.113 | 4.101 |
| ATOM | 55 | O4 | MAN | 4 | −0.132 | 29.729 | 28.611 | +0.12 | −0.38 | −0.537 | 4.101 |
| ATOM | 56 | HO4 | MAN | 4 | −0.351 | 30.620 | 29.078 | +0.11 | +0.25 | +0.424 | 4.101 |
| ATOM | 57 | C3 | MAN | 4 | −2.180 | 30.505 | 27.610 | −0.14 | +0.09 | +0.113 | 4.101 |
| ATOM | 58 | O3 | MAN | 4 | −2.806 | 30.863 | 28.833 | +0.18 | −0.41 | −0.537 | 4.101 |
| ATOM | 59 | HO3 | MAN | 4 | −2.708 | 31.878 | 28.986 | +0.11 | +0.28 | +0.424 | 4.101 |
| ATOM | 60 | C2 | MAN | 4 | −3.251 | 30.187 | 26.562 | −0.24 | +0.11 | +0.113 | 4.101 |
| ATOM | 61 | O2 | MAN | 4 | −4.166 | 29.232 | 27.077 | +0.02 | −0.61 | −0.537 | 4.101 |
| ATOM | 62 | HO2 | MAN | 4 | −4.209 | 29.317 | 28.104 | +0.10 | +0.44 | +0.424 | 4.101 |
| ATOM | 63 | C4 | MAN | 4 | −2.866 | 30.897 | 21.056 | −0.16 | +0.15 | +0.113 | 4.101 |
| ATOM | 64 | O4 | MAN | 4 | −2.991 | 32.297 | 20.853 | +0.15 | −0.58 | −0.537 | 4.101 |
| ATOM | 65 | HO4 | MAN | 4 | −3.865 | 32.632 | 21.286 | +0.11 | +0.48 | +0.424 | 4.101 |
| ATOM | 66 | C3 | MAN | 4 | −2.190 | 30.244 | 19.848 | −0.37 | +0.12 | +0.113 | 4.101 |
| ATOM | 67 | O3 | MAN | 4 | −2.853 | 30.559 | 18.592 | −0.01 | −0.28 | −0.227 | 4.101 |
| ATOM | 68 | C1 | BDG | 4 | −4.246 | 30.262 | 18.486 | −0.51 | +0.25 | +0.227 | 4.101 |
| ATOM | 69 | C2 | BDG | 4 | −4.625 | 30.044 | 17.003 | −0.57 | +0.12 | +0.113 | 4.101 |
| ATOM | 70 | O22 | BDG | 4 | −5.980 | 29.595 | 16.933 | −0.32 | −0.16 | −0.113 | 4.101 |
| ATOM | 71 | C3 | BDG | 4 | −4.500 | 31.360 | 16.198 | −0.63 | +0.14 | +0.113 | 4.101 |
| ATOM | 72 | O3 | BDG | 4 | −4.993 | 31.171 | 14.896 | −0.27 | −0.60 | −0.537 | 4.101 |
| ATOM | 73 | HO3 | BDG | 4 | −4.867 | 32.012 | 14.418 | −0.15 | +0.44 | +0.424 | 4.101 |
| ATOM | 74 | C4 | BDG | 4 | −5.249 | 32.539 | 16.873 | −0.44 | +0.13 | +0.113 | 4.101 |
| ATOM | 75 | O4 | BDG | 4 | −4.955 | 33.731 | 16.184 | −0.06 | −0.63 | −0.537 | 4.101 |
| ATOM | 76 | HO4 | BDG | 4 | −5.460 | 34.444 | 16.616 | +0.08 | +0.40 | +0.424 | 4.101 |
| ATOM | 77 | C5 | BDG | 4 | −4.860 | 32.655 | 18.377 | −0.27 | +0.12 | +0.113 | 4.101 |
| ATOM | 78 | C6 | BDG | 4 | −5.711 | 33.693 | 19.165 | −0.13 | +0.10 | +0.113 | 4.101 |
| ATOM | 79 | O6 | BDG | 4 | −5.468 | 35.007 | 18.712 | +0.16 | −0.39 | −0.537 | 4.101 |
| ATOM | 80 | H6 | BDG | 4 | −6.015 | 35.587 | 19.244 | +0.11 | +0.28 | +0.424 | 4.101 |
| ATOM | 81 | O | BDG | 4 | −5.013 | 31.372 | 19.035 | +0.10 | −0.26 | −0.227 | 4.101 |
| ATOM | 82 | C2 | MAN | 4 | −2.087 | 28.729 | 20.075 | −0.41 | +0.11 | +0.113 | 4.101 |
| ATOM | 83 | O2 | MAN | 4 | −3.390 | 28.172 | 20.176 | −0.16 | −0.51 | −0.537 | 4.101 |
| ATOM | 84 | HO2 | MAN | 4 | −3.336 | 27.148 | 20.080 | −0.04 | +0.29 | +0.424 | 4.101 |
| ATOM | 85 | C4 | MAN | 4 | 3.024 | 27.970 | 23.635 | −0.19 | +0.11 | +0.113 | 4.101 |
| ATOM | 86 | O4 | MAN | 4 | 2.275 | 27.527 | 24.758 | +0.09 | −0.51 | −0.537 | 4.101 |
| ATOM | 87 | HO4 | MAN | 4 | 1.306 | 27.330 | 24.469 | +0.10 | +0.38 | +0.424 | 4.101 |
| TER | | | | | | | | | | | |
| ENDMDL | | | | | | | | | | | |
| MODEL | 63 | | | | | | | | | | |
| USER | Run = 63 | | | | | | | | | | |
| USER | Cluster Rank = 5 | | | | | | | | | | |
| USER | Number of conformations in this cluster = 1 | | | | | | | | | | |
| USER | | | | | | | | | | | |
| USER | RMSD from reference structure = 3.770 A | | | | | | | | | | |
| USER | | | | | | | | | | | |
| USER | Estimated Free Energy of Binding = −1.15 kcal/mol [= (1) + (3)] | | | | | | | | | | |
| USER | Estimated Inhibition Constant, Ki = +0.14 [Temperature = 298.15 K] | | | | | | | | | | |
| USER | | | | | | | | | | | |
| USER | Final Docked Energy = −2.97 kcal/mol [= (1) + (2)] | | | | | | | | | | |
| USER | | | | | | | | | | | |
| USER | (1) Final Intermolecular Energy = −6.13 kcal/mol | | | | | | | | | | |
| USER | (2) Final Internal Energy of Ligand = +3.16 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 0.072417 31.451833 22.390484 | | | | | | | | | | |
| USER | NEWDPF quat0 0.769409 0.622464 −0.143347 −49.772934 | | | | | | | | | | |
| USER | NEWDPF ndihe 16 | | | | | | | | | | |
| USER | NEWDPF dihe0 40.95 −58.87 −72.81 −69.30 102.81 88.81 7.56 −36.16 −57.21 | | | | | | | | | | |
| | −141.46 60.70 106.53 −62.74 −47.54 7.24 −27.23 | | | | | | | | | | |
| USER | | | | | | | | | | | |
| USER | | | | Rank | x | y | z | vdW | Elec | q | RMS |
| ATOM | 1 | C5 | MAN | 5 | 4.025 | 27.161 | 26.192 | −0.35 | +0.09 | +0.113 | 3.770 |
| ATOM | 2 | HO4 | MAN | 5 | 3.639 | 29.626 | 27.162 | +0.11 | +0.23 | +0.424 | 3.770 |
| ATOM | 3 | O4 | MAN | 5 | 3.651 | 28.858 | 27.850 | +0.07 | −0.26 | −0.537 | 3.770 |
| ATOM | 4 | O5 | MAN | 5 | 3.370 | 26.144 | 25.398 | −0.03 | −0.31 | −0.227 | 3.770 |
| ATOM | 5 | HO2 | MAN | 5 | 0.165 | 25.224 | 26.223 | +0.03 | +0.49 | +0.424 | 3.770 |
| ATOM | 6 | O2 | MAN | 5 | 0.880 | 25.816 | 26.669 | −0.21 | −0.74 | −0.537 | 3.770 |
| ATOM | 7 | C2 | MAN | 5 | 1.142 | 26.937 | 25.847 | −0.44 | +0.11 | +0.113 | 3.770 |
| ATOM | 8 | C1 | MAN | 5 | 2.154 | 26.567 | 24.778 | −0.30 | +0.27 | +0.227 | 3.770 |
| ATOM | 9 | C4 | MAN | 5 | 3.074 | 27.699 | 27.274 | −0.40 | +0.07 | +0.113 | 3.770 |
| ATOM | 10 | O3 | MAN | 5 | 0.800 | 28.371 | 27.741 | +0.05 | −0.48 | −0.537 | 3.770 |
| ATOM | 11 | HO3 | MAN | 5 | −0.082 | 28.727 | 27.346 | +0.11 | +0.39 | +0.424 | 3.770 |
| ATOM | 12 | C3 | MAN | 5 | 1.704 | 28.072 | 26.691 | −0.29 | +0.09 | +0.113 | 3.770 |
| ATOM | 13 | C6 | MAN | 5 | 5.272 | 26.529 | 26.787 | −0.41 | +0.06 | +0.113 | 3.770 |
| ATOM | 14 | O6 | MAN | 5 | 5.348 | 25.142 | 26.482 | −0.13 | −0.36 | −0.537 | 3.770 |
| ATOM | 15 | H6 | MAN | 5 | 6.213 | 24.952 | 25.958 | +0.06 | +0.31 | +0.424 | 3.770 |
| ATOM | 16 | O3 | MAN | 5 | 2.346 | 27.676 | 23.960 | +0.10 | −0.22 | −0.227 | 3.770 |
| ATOM | 17 | C3 | MAN | 5 | 2.175 | 27.443 | 22.559 | −0.21 | +0.09 | +0.113 | 3.770 |
| ATOM | 18 | C2 | MAN | 5 | 3.429 | 27.942 | 21.850 | −0.17 | +0.10 | +0.113 | 3.770 |
| ATOM | 19 | O2 | MAN | 5 | 3.752 | 29.253 | 22.296 | +0.21 | −0.42 | −0.537 | 3.770 |
| ATOM | 20 | HO2 | MAN | 5 | 4.214 | 29.770 | 21.534 | +0.12 | +0.32 | +0.424 | 3.770 |
| ATOM | 21 | C1 | MAN | 5 | 3.212 | 27.953 | 20.355 | −0.23 | +0.18 | +0.227 | 3.770 |
| ATOM | 22 | O4 | NAG | 5 | 4.325 | 28.486 | 19.734 | +0.17 | −0.19 | −0.227 | 3.770 |
| ATOM | 23 | C4 | NAG | 5 | 5.328 | 27.572 | 19.272 | −0.14 | +0.10 | +0.113 | 3.770 |
| ATOM | 24 | C5 | NAG | 5 | 6.642 | 27.861 | 20.028 | −0.05 | +0.10 | +0.113 | 3.770 |
| ATOM | 25 | C6 | NAG | 5 | 6.663 | 27.343 | 21.457 | −0.06 | +0.10 | +0.113 | 3.770 |
| ATOM | 26 | O6 | NAG | 5 | 7.617 | 26.304 | 21.629 | +0.21 | −0.47 | −0.537 | 3.770 |
| ATOM | 27 | H6 | NAG | 5 | 8.387 | 26.429 | 20.955 | +0.12 | +0.35 | +0.424 | 3.770 |
| ATOM | 28 | O5 | NAG | 5 | 7.736 | 27.232 | 19.374 | +0.22 | −0.20 | −0.227 | 3.770 |
| ATOM | 29 | C1 | NAG | 5 | 7.919 | 27.712 | 18.057 | −0.10 | +0.11 | +0.113 | 3.770 |
| ATOM | 30 | C2 | NAG | 5 | 6.754 | 27.174 | 17.211 | −0.23 | +0.22 | +0.211 | 3.770 |
| ATOM | 31 | N2 | NAG | 5 | 6.889 | 27.550 | 15.821 | −0.26 | −0.82 | −0.650 | 3.770 |
| ATOM | 32 | HN2 | NAG | 5 | 6.675 | 28.513 | 15.528 | +0.12 | +0.67 | +0.440 | 3.770 |
| ATOM | 33 | C7 | NAG | 5 | 7.289 | 26.657 | 14.919 | −0.45 | +0.55 | +0.396 | 3.770 |
| ATOM | 34 | O7 | NAG | 5 | 7.559 | 25.491 | 15.205 | −0.10 | −0.60 | −0.396 | 3.770 |
| ATOM | 35 | C8 | NAG | 5 | 7.414 | 27.127 | 13.481 | −0.57 | +0.00 | +0.000 | 3.770 |
| ATOM | 36 | C3 | NAG | 5 | 5.456 | 27.755 | 17.759 | −0.26 | +0.11 | +0.113 | 3.770 |
| ATOM | 37 | O3 | NAG | 5 | 4.346 | 27.139 | 17.124 | −0.03 | −0.54 | −0.537 | 3.770 |
| ATOM | 38 | HO3 | NAG | 5 | 4.253 | 26.169 | 17.457 | +0.09 | +0.43 | +0.424 | 3.770 |
| ATOM | 39 | O5 | MAN | 5 | 2.060 | 28.749 | 20.038 | +0.07 | −0.17 | −0.227 | 3.770 |
| ATOM | 40 | C5 | MAN | 5 | 0.860 | 28.145 | 20.530 | −0.38 | +0.07 | +0.113 | 3.770 |
| ATOM | 41 | C6 | MAN | 5 | −0.334 | 28.803 | 19.825 | −0.15 | +0.09 | +0.113 | 3.770 |
| ATOM | 42 | O6 | MAN | 5 | −0.825 | 29.955 | 20.557 | +0.08 | −0.21 | −0.227 | 3.770 |
| ATOM | 43 | C1 | MAN | 5 | −0.044 | 31.127 | 20.432 | −0.22 | +0.22 | +0.227 | 3.770 |
| ATOM | 44 | O5 | MAN | 5 | −0.748 | 32.268 | 19.910 | +0.01 | −0.27 | −0.227 | 3.770 |
| ATOM | 45 | C5 | MAN | 5 | −1.680 | 32.901 | 20.824 | −0.16 | +0.10 | +0.113 | 3.770 |
| ATOM | 46 | C6 | MAN | 5 | −2.399 | 34.025 | 20.084 | −0.21 | +0.09 | +0.113 | 3.770 |
| ATOM | 47 | O6 | MAN | 5 | −3.146 | 33.500 | 18.969 | +0.07 | −0.23 | −0.227 | 3.770 |
| ATOM | 48 | C1 | MAN | 5 | −4.388 | 32.961 | 19.345 | −0.20 | +0.23 | +0.227 | 3.770 |
| ATOM | 49 | O5 | MAN | 5 | −5.372 | 33.073 | 18.313 | +0.14 | −0.22 | −0.227 | 3.770 |
| ATOM | 50 | C5 | MAN | 5 | −5.023 | 32.407 | 17.076 | −0.40 | +0.13 | +0.113 | 3.770 |
| ATOM | 51 | C6 | MAN | 5 | −6.172 | 32.620 | 16.112 | −0.51 | +0.12 | +0.113 | 3.770 |
| ATOM | 52 | O6 | MAN | 5 | −5.845 | 32.158 | 14.809 | −0.14 | −0.60 | −0.537 | 3.770 |
| ATOM | 53 | H6 | MAN | 5 | −4.855 | 32.362 | 14.610 | −0.23 | +0.47 | +0.424 | 3.770 |
| ATOM | 54 | C4 | MAN | 5 | −4.762 | 30.915 | 17.313 | −0.57 | +0.13 | +0.113 | 3.770 |
| ATOM | 55 | O4 | MAN | 5 | −4.210 | 30.362 | 16.130 | −0.08 | −0.66 | −0.537 | 3.770 |
| ATOM | 56 | HO4 | MAN | 5 | −3.471 | 29.690 | 16.378 | +0.07 | +0.53 | +0.424 | 3.770 |
| ATOM | 57 | C3 | MAN | 5 | −3.799 | 30.677 | 18.482 | −0.47 | +0.13 | +0.113 | 3.770 |
| ATOM | 58 | O3 | MAN | 5 | −3.776 | 29.295 | 18.806 | −0.10 | −0.53 | −0.537 | 3.770 |
| ATOM | 59 | HO3 | MAN | 5 | −2.799 | 28.971 | 18.863 | +0.05 | +0.45 | +0.424 | 3.770 |
| ATOM | 60 | C2 | MAN | 5 | −4.205 | 31.486 | 19.719 | −0.27 | +0.13 | +0.113 | 3.770 |
| ATOM | 61 | O2 | MAN | 5 | −5.411 | 30.969 | 20.261 | −0.06 | −0.79 | −0.537 | 3.770 |
| ATOM | 62 | HO2 | MAN | 5 | −5.487 | 29.966 | 20.037 | +0.08 | +0.57 | +0.424 | 3.770 |
| ATOM | 63 | C4 | MAN | 5 | −0.948 | 33.367 | 22.087 | −0.07 | +0.09 | +0.113 | 3.770 |
| ATOM | 64 | O4 | MAN | 5 | −1.862 | 33.993 | 22.976 | +0.22 | −0.38 | −0.537 | 3.770 |
| ATOM | 65 | HO4 | MAN | 5 | −1.816 | 35.015 | 22.854 | +0.12 | +0.26 | +0.424 | 3.770 |
| ATOM | 66 | C3 | MAN | 5 | −0.352 | 32.114 | 22.734 | −0.06 | +0.09 | +0.113 | 3.770 |
| ATOM | 67 | O3 | MAN | 5 | 0.243 | 32.380 | 24.035 | +0.23 | −0.16 | −0.227 | 3.770 |
| ATOM | 68 | C1 | BDG | 5 | 0.249 | 31.316 | 24.988 | −0.04 | +0.17 | +0.227 | 3.770 |
| ATOM | 69 | C2 | BDG | 5 | −1.149 | 30.655 | 25.045 | −0.11 | +0.10 | +0.113 | 3.770 |
| ATOM | 70 | O22 | BDG | 5 | −1.080 | 29.499 | 25.882 | +0.16 | −0.11 | −0.113 | 3.770 |
| ATOM | 71 | C3 | BDG | 5 | −2.200 | 31.627 | 25.631 | −0.10 | +0.10 | +0.113 | 3.770 |
| ATOM | 72 | O3 | BDG | 5 | −3.422 | 30.956 | 25.811 | +0.12 | −0.54 | −0.537 | 3.770 |
| ATOM | 73 | HO3 | BDG | 5 | −4.060 | 31.610 | 26.152 | +0.10 | +0.39 | +0.424 | 3.770 |
| ATOM | 74 | C4 | BDG | 5 | −1.744 | 32.250 | 26.975 | −0.04 | +0.08 | +0.113 | 3.770 |
| ATOM | 75 | O4 | BDG | 5 | −2.664 | 33.243 | 27.364 | +0.23 | −0.35 | −0.537 | 3.770 |
| ATOM | 76 | HO4 | BDG | 5 | −2.362 | 33.594 | 28.221 | +0.12 | +0.25 | +0.424 | 3.770 |
| ATOM | 77 | C5 | BDG | 5 | −0.307 | 32.840 | 26.854 | −0.02 | +0.07 | +0.113 | 3.770 |
| ATOM | 78 | C6 | BDG | 5 | 0.291 | 33.312 | 28.211 | −0.04 | +0.06 | +0.113 | 3.770 |
| ATOM | 79 | O6 | BDG | 5 | −0.419 | 34.416 | 28.730 | +0.23 | −0.28 | −0.537 | 3.770 |
| ATOM | 80 | H6 | BDG | 5 | 0.008 | 34.655 | 29.555 | +0.12 | +0.21 | +0.424 | 3.770 |
| ATOM | 81 | O | BDG | 5 | 0.596 | 31.851 | 26.298 | +0.23 | −0.15 | −0.227 | 3.770 |
| ATOM | 82 | C2 | MAN | 5 | 0.652 | 31.466 | 21.768 | −0.08 | +0.09 | +0.113 | 3.770 |
| ATOM | 83 | O2 | MAN | 5 | 1.726 | 32.363 | 21.525 | +0.21 | −0.40 | −0.537 | 3.770 |
| ATOM | 84 | HO2 | MAN | 5 | 2.508 | 31.854 | 21.089 | +0.12 | +0.32 | +0.424 | 3.770 |
| ATOM | 85 | C4 | MAN | 5 | 0.891 | 28.152 | 22.065 | −0.27 | +0.08 | +0.113 | 3.770 |
| ATOM | 86 | O4 | MAN | 5 | −0.251 | 27.471 | 22.567 | +0.03 | −0.37 | −0.537 | 3.770 |
| ATOM | 87 | HO4 | MAN | 5 | −0.844 | 27.169 | 21.781 | +0.06 | +0.27 | +0.424 | 3.770 |
| TER | | | | | | | | | | | |
| ENDMDL | | | | | | | | | | | |
| MODEL | 49 | | | | | | | | | | |
| USER | Run = 49 | | | | | | | | | | |
| USER | Cluster Rank = 6 | | | | | | | | | | |
| USER | Number of conformations in this cluster = 1 | | | | | | | | | | |
| USER | | | | | | | | | | | |
| USER | RMSD from reference structure = 3.587 A | | | | | | | | | | |
| USER | | | | | | | | | | | |
| USER | Estimated Free Energy of Binding = −4.81 kcal/mol [= (1) + (3)] | | | | | | | | | | |
| USER | Estimated Inhibition Constant, Ki = +2.98e−04 [Temperature = 298.15 K] | | | | | | | | | | |
| USER | | | | | | | | | | | |
| USER | Final Docked Energy = −2.93 kcal/mol [= (1) + (2)] | | | | | | | | | | |
| USER | | | | | | | | | | | |
| USER | (1) Final Intermolecular Energy = −9.79 kcal/mol | | | | | | | | | | |
| USER | (2) Final Internal Energy of Ligand = +6.87 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 3.639617 29.106320 23.969369 | | | | | | | | | | |
| USER | NEWDPF quat0 −0.269301 0.698560 0.662941 41.537230 | | | | | | | | | | |
| USER | NEWDPF ndihe 16 | | | | | | | | | | |
| USER | NEWDPF dihe0 −52.58 −114.10 167.58 139.08 7.77 49.39 171.09 −50.01 | | | | | | | | | | |
| | 111.44 −168.95 2.16 167.80 133.01 −140.85 73.32 −30.14 | | | | | | | | | | |
| USER | | | | | | | | | | | |
| USER | | | | Rank | x | y | z | vdW | Elec | q | RMS |
| ATOM | 1 | C5 | MAN | 6 | 8.442 | 25.272 | 20.695 | −0.10 | +0.10 | +0.113 | 3.587 |
| ATOM | 2 | HO4 | MAN | 6 | 9.326 | 27.366 | 22.109 | +0.12 | +0.30 | +0.424 | 3.587 |
| ATOM | 3 | O4 | MAN | 6 | 9.906 | 26.515 | 22.137 | +0.22 | −0.38 | −0.537 | 3.587 |
| ATOM | 4 | O5 | MAN | 6 | 7.385 | 24.285 | 20.679 | +0.15 | −0.22 | −0.227 | 3.587 |
| ATOM | 5 | HO2 | MAN | 6 | 6.756 | 22.503 | 23.549 | −0.20 | +0.26 | +0.424 | 3.587 |
| ATOM | 6 | O2 | MAN | 6 | 7.493 | 23.160 | 23.257 | −0.01 | −0.38 | −0.537 | 3.587 |
| ATOM | 7 | C2 | MAN | 6 | 6.928 | 24.437 | 23.038 | −0.30 | +0.13 | +0.113 | 3.587 |
| ATOM | 8 | C1 | MAN | 6 | 6.348 | 24.510 | 21.637 | −0.19 | +0.31 | +0.227 | 3.587 |
| ATOM | 9 | C4 | MAN | 6 | 9.070 | 25.372 | 22.095 | −0.10 | +0.09 | +0.113 | 3.587 |
| ATOM | 10 | O3 | MAN | 6 | 8.603 | 25.395 | 24.466 | +0.13 | −0.35 | −0.537 | 3.587 |
| ATOM | 11 | HO3 | MAN | 6 | 7.903 | 25.595 | 25.195 | +0.10 | +0.28 | +0.424 | 3.587 |
| ATOM | 12 | C3 | MAN | 6 | 8.000 | 25.507 | 23.188 | −0.17 | +0.09 | +0.113 | 3.587 |
| ATOM | 13 | C6 | MAN | 6 | 9.455 | 24.858 | 19.641 | −0.17 | +0.10 | +0.113 | 3.587 |
| ATOM | 14 | O6 | MAN | 6 | 9.997 | 23.573 | 19.917 | +0.05 | −0.48 | −0.537 | 3.587 |
| ATOM | 15 | H6 | MAN | 6 | 9.317 | 23.022 | 20.459 | +0.08 | +0.37 | +0.424 | 3.587 |
| ATOM | 16 | O3 | MAN | 6 | 5.756 | 25.760 | 21.477 | +0.12 | −0.31 | −0.227 | 3.587 |
| ATOM | 17 | C3 | MAN | 6 | 4.419 | 25.745 | 20.970 | +0.09 | +0.16 | +0.113 | 3.587 |
| ATOM | 18 | C2 | MAN | 6 | 4.454 | 25.095 | 19.591 | −0.26 | +0.12 | +0.113 | 3.587 |
| ATOM | 19 | O2 | MAN | 6 | 5.483 | 25.680 | 18.804 | +0.16 | −0.53 | −0.537 | 3.587 |
| ATOM | 20 | HO2 | MAN | 6 | 5.212 | 25.653 | 17.810 | +0.10 | +0.45 | +0.424 | 3.587 |
| ATOM | 21 | C1 | MAN | 6 | 3.123 | 25.271 | 18.898 | −0.39 | +0.17 | +0.227 | 3.587 |
| ATOM | 22 | O4 | NAG | 6 | 3.198 | 24.751 | 17.621 | −0.01 | −0.21 | −0.227 | 3.587 |
| ATOM | 23 | C4 | NAG | 6 | 3.369 | 23.334 | 17.489 | −0.40 | +0.10 | +0.113 | 3.587 |
| ATOM | 24 | C5 | NAG | 6 | 4.514 | 23.067 | 16.489 | −0.27 | +0.14 | +0.113 | 3.587 |
| ATOM | 25 | C6 | NAG | 6 | 5.903 | 23.299 | 17.061 | −0.33 | +0.13 | +0.113 | 3.587 |
| ATOM | 26 | O6 | NAG | 6 | 6.647 | 22.090 | 17.151 | +0.04 | −0.54 | −0.537 | 3.587 |
| ATOM | 27 | H6 | NAG | 6 | 6.328 | 21.436 | 16.422 | +0.04 | +0.38 | +0.424 | 3.587 |
| ATOM | 28 | O5 | NAG | 6 | 4.493 | 21.711 | 16.067 | +0.11 | −0.19 | −0.227 | 3.587 |
| ATOM | 29 | C1 | NAG | 6 | 3.293 | 21.379 | 15.397 | −0.35 | +0.09 | +0.113 | 3.587 |
| ATOM | 30 | C2 | NAG | 6 | 2.177 | 21.348 | 16.453 | −0.38 | +0.10 | +0.211 | 3.587 |
| ATOM | 31 | N2 | NAG | 6 | 0.913 | 20.943 | 15.879 | −0.27 | −0.25 | −0.650 | 3.587 |
| ATOM | 32 | HN2 | NAG | 6 | 0.344 | 20.224 | 16.346 | −0.01 | −0.02 | +0.440 | 3.587 |
| ATOM | 33 | C7 | NAG | 6 | 0.481 | 21.496 | 14.748 | −0.65 | +0.34 | −0.396 | 3.587 |
| ATOM | 34 | O7 | NAG | 6 | 1.113 | 22.360 | 14.141 | +0.06 | −0.43 | −0.396 | 3.587 |
| ATOM | 35 | C8 | NAG | 6 | −0.858 | 21.026 | 14.209 | −0.49 | +0.00 | +0.000 | 3.587 |
| ATOM | 36 | C3 | NAG | 6 | 2.031 | 22.749 | 17.036 | −0.50 | +0.06 | +0.113 | 3.587 |
| ATOM | 37 | O3 | NAG | 6 | 1.141 | 22.722 | 18.142 | +0.18 | +0.06 | −0.537 | 3.587 |
| ATOM | 38 | HO3 | NAG | 6 | 1.594 | 22.251 | 18.937 | −0.09 | −0.05 | +0.424 | 3.587 |
| ATOM | 39 | O5 | MAN | 6 | 2.793 | 26.666 | 18.829 | +0.01 | −0.17 | −0.227 | 3.587 |
| ATOM | 40 | C5 | MAN | 6 | 2.549 | 27.207 | 20.132 | −0.34 | +0.08 | +0.113 | 3.587 |
| ATOM | 41 | C6 | MAN | 6 | 1.820 | 28.548 | 19.966 | −0.33 | +0.08 | +0.113 | 3.587 |
| ATOM | 42 | O6 | MAN | 6 | 1.456 | 29.127 | 21.246 | +0.15 | −0.17 | −0.227 | 3.587 |
| ATOM | 43 | C1 | MAN | 6 | 0.209 | 28.710 | 21.763 | −0.27 | +0.18 | +0.227 | 3.587 |
| ATOM | 44 | O5 | MAN | 6 | −0.175 | 29.343 | 22.996 | +0.16 | −0.20 | −0.227 | 3.587 |
| ATOM | 45 | C5 | MAN | 6 | −0.603 | 30.725 | 22.884 | −0.11 | +0.10 | +0.113 | 3.587 |
| ATOM | 46 | C6 | MAN | 6 | −0.881 | 31.263 | 24.285 | −0.07 | +0.10 | +0.113 | 3.587 |
| ATOM | 47 | O6 | MAN | 6 | −0.574 | 30.267 | 25.280 | +0.20 | −0.19 | −0.227 | 3.587 |
| ATOM | 48 | C1 | MAN | 6 | −1.717 | 29.756 | 25.916 | −0.20 | +0.23 | +0.227 | 3.587 |
| ATOM | 49 | O5 | MAN | 6 | −1.981 | 28.395 | 25.565 | −0.03 | −0.28 | −0.227 | 3.587 |
| ATOM | 50 | C5 | MAN | 6 | −0.914 | 27.473 | 25.894 | −0.43 | +0.12 | +0.113 | 3.587 |
| ATOM | 51 | C6 | MAN | 6 | −1.365 | 26.092 | 25.464 | −0.71 | +0.10 | +0.113 | 3.587 |
| ATOM | 52 | O6 | MAN | 6 | −1.789 | 26.086 | 24.109 | −0.07 | −0.42 | −0.537 | 3.587 |
| ATOM | 53 | H6 | MAN | 6 | −2.284 | 26.965 | 23.899 | +0.05 | +0.53 | +0.424 | 3.587 |
| ATOM | 54 | C4 | MAN | 6 | −0.597 | 27.516 | 27.393 | −0.41 | +0.14 | +0.113 | 3.587 |
| ATOM | 55 | O4 | MAN | 6 | 0.589 | 26.775 | 27.623 | −0.15 | −0.96 | −0.537 | 3.587 |
| ATOM | 56 | HO4 | MAN | 6 | 1.167 | 27.260 | 28.325 | +0.07 | +0.56 | +0.424 | 3.587 |
| ATOM | 57 | C3 | MAN | 6 | −0.407 | 28.950 | 27.901 | −0.30 | +0.10 | +0.113 | 3.587 |
| ATOM | 58 | O3 | MAN | 6 | −0.351 | 28.949 | 29.320 | −0.02 | −0.42 | −0.537 | 3.587 |
| ATOM | 59 | HO3 | MAN | 6 | 0.472 | 29.485 | 29.632 | +0.07 | +0.21 | +0.424 | 3.587 |
| ATOM | 60 | C2 | MAN | 6 | −1.543 | 29.867 | 27.435 | −0.18 | +0.10 | +0.113 | 3.587 |
| ATOM | 61 | O2 | MAN | 6 | −2.752 | 29.513 | 28.090 | +0.10 | −0.53 | −0.537 | 3.587 |
| ATOM | 62 | HO2 | MAN | 6 | −2.536 | 29.093 | 29.006 | +0.09 | +0.44 | +0.424 | 3.587 |
| ATOM | 63 | C4 | MAN | 6 | −1.801 | 30.833 | 21.936 | −0.17 | +0.13 | +0.113 | 3.587 |
| ATOM | 64 | O4 | MAN | 6 | −2.221 | 32.186 | 21.834 | +0.17 | −0.54 | −0.537 | 3.587 |
| ATOM | 65 | HO4 | MAN | 6 | −3.011 | 32.350 | 22.475 | +0.10 | +0.46 | +0.424 | 3.587 |
| ATOM | 66 | C3 | MAN | 6 | −1.327 | 30.321 | 20.573 | −0.29 | +0.12 | +0.113 | 3.587 |
| ATOM | 67 | O3 | MAN | 6 | −2.316 | 30.510 | 19.524 | +0.03 | −0.26 | −0.227 | 3.587 |
| ATOM | 68 | C1 | BDG | 6 | −3.668 | 30.152 | 19.817 | −0.40 | +0.27 | +0.227 | 3.587 |
| ATOM | 69 | C2 | BDG | 6 | −4.319 | 29.504 | 18.574 | −0.53 | +0.11 | +0.113 | 3.587 |
| ATOM | 70 | O22 | BDG | 6 | −5.612 | 29.011 | 18.933 | −0.11 | −0.11 | −0.113 | 3.587 |
| ATOM | 71 | C3 | BDG | 6 | −4.485 | 30.534 | 17.431 | −0.60 | +0.13 | +0.113 | 3.587 |
| ATOM | 72 | O3 | BDG | 6 | −5.213 | 29.961 | 16.374 | −0.13 | −0.64 | −0.537 | 3.587 |
| ATOM | 73 | HO3 | BDG | 6 | −5.267 | 30.631 | 15.668 | +0.03 | +0.50 | +0.424 | 3.587 |
| ATOM | 74 | C4 | BDG | 6 | −5.187 | 31.833 | 17.904 | −0.36 | +0.13 | +0.113 | 3.587 |
| ATOM | 75 | O4 | BDG | 6 | −5.153 | 32.784 | 16.866 | +0.02 | −0.62 | −0.537 | 3.587 |
| ATOM | 76 | HO4 | BDG | 6 | −5.622 | 33.575 | 17.187 | +0.10 | +0.42 | +0.424 | 3.587 |
| ATOM | 77 | C5 | BDG | 6 | −4.511 | 32.391 | 19.193 | −0.24 | +0.12 | +0.113 | 3.587 |
| ATOM | 78 | C6 | BDG | 6 | −5.277 | 33.585 | 19.833 | −0.12 | +0.10 | +0.113 | 3.587 |
| ATOM | 79 | O6 | BDG | 6 | −5.255 | 34.720 | 18.993 | +0.17 | −0.40 | −0.537 | 3.587 |
| ATOM | 80 | H6 | BDG | 6 | −5.734 | 35.411 | 19.454 | +0.11 | +0.28 | +0.424 | 3.587 |
| ATOM | 81 | O | BDG | 6 | −4.407 | 31.349 | 20.195 | +0.02 | −0.30 | −0.227 | 3.587 |
| ATOM | 82 | C2 | MAN | 6 | −0.900 | 28.851 | 20.697 | −0.39 | +0.10 | +0.113 | 3.587 |
| ATOM | 83 | O2 | MAN | 6 | −2.019 | 28.061 | 21.075 | −0.09 | −0.52 | −0.537 | 3.587 |
| ATOM | 84 | HO2 | MAN | 6 | −1.806 | 27.065 | 20.921 | +0.05 | +0.30 | +0.424 | 3.587 |
| ATOM | 85 | C4 | MAN | 6 | 3.857 | 27.187 | 20.933 | −0.21 | +0.11 | +0.113 | 3.587 |
| ATOM | 86 | O4 | MAN | 6 | 3.610 | 27.634 | 22.259 | +0.13 | −0.54 | −0.537 | 3.587 |
| ATOM | 87 | HO4 | MAN | 6 | 2.619 | 27.900 | 22.353 | +0.10 | +0.36 | +0.424 | 3.587 |
| TER | | | | | | | | | | | |
| ENDMDL | | | | | | | | | | | |
| MODEL | 47 | | | | | | | | | | |
| USER | Run = 47 | | | | | | | | | | |
| USER | Cluster Rank = 7 | | | | | | | | | | |
| USER | Number of conformations in this cluster = 1 | | | | | | | | | | |
| USER | | | | | | | | | | | |
| USER | RMSD from reference structure = 3.664 A | | | | | | | | | | |
| USER | | | | | | | | | | | |
| USER | Estimated Free Energy of Binding = +0.18 kcal/mol [= (1) + (3)] | | | | | | | | | | |
| USER | Estimated Inhibition Constant, Ki = +1.35 [Temperature = 298.15 K] | | | | | | | | | | |
| USER | | | | | | | | | | | |
| USER | Final Docked Energy = −2.64 kcal/mol [= (1) + (2)] | | | | | | | | | | |
| USER | | | | | | | | | | | |
| USER | (1) Final Intermolecular Energy = −4.80 kcal/mol | | | | | | | | | | |
| USER | (2) Final Internal Energy of Ligand = +2.16 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 0.067706 31.212406 16.183295 | | | | | | | | | | |
| USER | NEWDPF quat0 −0.516215 −0.855680 0.036514 168.292099 | | | | | | | | | | |
| USER | NEWDPF ndihe 16 | | | | | | | | | | |
| USER | NEWDPF dihe0 −180.00 46.15 −155.29 −50.59 −0.37 26.15 25.24 −45.23 | | | | | | | | | | |
| | 122.92 −45.12 151.09 120.60 −48.82 124.46 −37.97 −52.70 | | | | | | | | | | |
| USER | | | | | | | | | | | |
| USER | | | | Rank | x | y | z | vdW | Elec | q | RMS |
| ATOM | 1 | C5 | MAN | 7 | −6.007 | 31.943 | 19.509 | −0.20 | +0.13 | +0.113 | 3.664 |
| ATOM | 2 | HO4 | MAN | 7 | −5.691 | 33.341 | 17.247 | +0.10 | +0.42 | +0.424 | 3.664 |
| ATOM | 3 | O4 | MAN | 7 | −6.627 | 32.944 | 17.416 | +0.12 | −0.53 | −0.537 | 3.664 |
| ATOM | 4 | O5 | MAN | 7 | −5.610 | 30.700 | 20.134 | −0.04 | −0.32 | −0.227 | 3.664 |
| ATOM | 5 | HO2 | MAN | 7 | −6.005 | 27.776 | 18.374 | −0.24 | +0.30 | +0.424 | 3.664 |
| ATOM | 6 | O2 | MAN | 7 | −6.297 | 28.752 | 18.222 | −0.16 | −0.59 | −0.537 | 3.664 |
| ATOM | 7 | C2 | MAN | 7 | −5.152 | 29.566 | 18.060 | −0.52 | +0.11 | +0.113 | 3.664 |
| ATOM | 8 | C1 | MAN | 7 | −4.610 | 29.970 | 19.420 | −0.46 | +0.26 | +0.227 | 3.664 |
| ATOM | 9 | C4 | MAN | 7 | −6.500 | 31.696 | 18.074 | −0.31 | +0.12 | +0.113 | 3.664 |
| ATOM | 10 | O3 | MAN | 7 | −6.101 | 30.452 | 16.041 | −0.17 | −0.74 | −0.537 | 3.664 |
| ATOM | 11 | HO3 | MAN | 7 | −5.403 | 29.960 | 15.466 | +0.03 | +0.48 | +0.424 | 3.664 |
| ATOM | 12 | C3 | MAN | 7 | −5.519 | 30.820 | 17.280 | −0.57 | +0.13 | +0.113 | 3.664 |
| ATOM | 13 | C6 | MAN | 7 | −7.082 | 32.561 | 20.387 | −0.13 | +0.12 | +0.113 | 3.664 |
| ATOM | 14 | O6 | MAN | 7 | −6.550 | 33.585 | 21.217 | +0.20 | −0.51 | −0.537 | 3.664 |
| ATOM | 15 | H6 | MAN | 7 | −6.398 | 33.215 | 22.165 | +0.11 | +0.46 | +0.424 | 3.664 |
| ATOM | 16 | O3 | MAN | 7 | −3.457 | 30.724 | 19.220 | +0.03 | −0.26 | −0.227 | 3.664 |
| ATOM | 17 | C3 | MAN | 7 | −2.501 | 30.674 | 20.282 | −0.31 | +0.13 | +0.113 | 3.664 |
| ATOM | 18 | C2 | MAN | 7 | −1.416 | 31.702 | 19.975 | −0.28 | +0.13 | +0.113 | 3.664 |
| ATOM | 19 | O2 | MAN | 7 | −0.966 | 31.550 | 18.635 | −0.10 | −0.90 | −0.537 | 3.664 |
| ATOM | 20 | HO2 | MAN | 7 | 0.024 | 31.827 | 18.572 | +0.06 | +0.83 | +0.424 | 3.664 |
| ATOM | 21 | C1 | MAN | 7 | −0.252 | 31.528 | 20.924 | −0.16 | +0.21 | +0.227 | 3.664 |
| ATOM | 22 | O4 | NAG | 7 | 0.755 | 32.409 | 20.580 | +0.16 | −0.20 | −0.227 | 3.664 |
| ATOM | 23 | C4 | NAG | 7 | 2.029 | 32.242 | 21.216 | −0.08 | +0.09 | +0.113 | 3.664 |
| ATOM | 24 | C5 | NAG | 7 | 2.445 | 33.587 | 21.847 | −0.04 | +0.07 | +0.113 | 3.664 |
| ATOM | 25 | C6 | NAG | 7 | 1.714 | 33.919 | 23.138 | −0.01 | +0.07 | +0.113 | 3.664 |
| ATOM | 26 | O6 | NAG | 7 | 2.593 | 33.928 | 24.255 | +0.24 | −0.31 | −0.537 | 3.664 |
| ATOM | 27 | H6 | NAG | 7 | 3.542 | 34.181 | 23.944 | +0.12 | +0.24 | +0.424 | 3.664 |
| ATOM | 28 | O5 | NAG | 7 | 3.826 | 33.570 | 22.179 | +0.23 | −0.14 | −0.227 | 3.664 |
| ATOM | 29 | C1 | NAG | 7 | 4.647 | 33.392 | 21.042 | −0.03 | +0.08 | +0.113 | 3.664 |
| ATOM | 30 | C2 | NAG | 7 | 4.465 | 31.939 | 20.573 | −0.06 | +0.15 | +0.211 | 3.664 |
| ATOM | 31 | N2 | NAG | 7 | 5.324 | 31.632 | 19.451 | −0.06 | −0.51 | −0.650 | 3.664 |
| ATOM | 32 | HN2 | NAG | 7 | 5.265 | 32.194 | 18.591 | +0.10 | +0.37 | +0.440 | 3.664 |
| ATOM | 33 | C7 | NAG | 7 | 6.193 | 30.626 | 19.526 | −0.08 | +0.32 | +0.396 | 3.664 |
| ATOM | 34 | O7 | NAG | 7 | 6.313 | 29.918 | 20.524 | +0.22 | −0.30 | −0.396 | 3.664 |
| ATOM | 35 | C8 | NAG | 7 | 7.067 | 30.368 | 18.312 | −0.14 | +0.00 | +0.000 | 3.664 |
| ATOM | 36 | C3 | NAG | 7 | 3.013 | 31.750 | 20.153 | −0.12 | +0.09 | +0.113 | 3.664 |
| ATOM | 37 | O3 | NAG | 7 | 2.761 | 30.378 | 19.889 | +0.15 | −0.43 | −0.537 | 3.664 |
| ATOM | 38 | HO3 | NAG | 7 | 2.753 | 29.853 | 20.775 | +0.11 | +0.33 | +0.424 | 3.664 |
| ATOM | 39 | O5 | MAN | 7 | 0.241 | 30.182 | 20.845 | +0.13 | −0.19 | −0.227 | 3.664 |
| ATOM | 40 | C5 | MAN | 7 | −0.720 | 29.247 | 21.345 | −0.30 | +0.10 | +0.113 | 3.664 |
| ATOM | 41 | C6 | MAN | 7 | −0.002 | 27.917 | 21.614 | −0.37 | +0.08 | +0.113 | 3.664 |
| ATOM | 42 | O6 | MAN | 7 | 0.683 | 27.922 | 22.893 | +0.08 | −0.17 | −0.227 | 3.664 |
| ATOM | 43 | C1 | MAN | 7 | −0.080 | 27.455 | 23.987 | −0.37 | +0.19 | +0.227 | 3.664 |
| ATOM | 44 | O5 | MAN | 7 | 0.618 | 26.561 | 24.871 | −0.04 | −0.21 | −0.227 | 3.664 |
| ATOM | 45 | C5 | MAN | 7 | 1.608 | 27.179 | 25.733 | −0.38 | +0.11 | +0.113 | 3.664 |
| ATOM | 46 | C6 | MAN | 7 | 2.311 | 26.083 | 26.530 | −0.10 | +0.11 | +0.113 | 3.664 |
| ATOM | 47 | O6 | MAN | 7 | 3.653 | 26.483 | 26.869 | +0.03 | −0.14 | −0.227 | 3.664 |
| ATOM | 48 | C1 | MAN | 7 | 4.621 | 25.948 | 26.003 | −0.39 | +0.22 | +0.227 | 3.664 |
| ATOM | 49 | O5 | MAN | 7 | 4.839 | 26.763 | 24.848 | −0.20 | −0.28 | −0.227 | 3.664 |
| ATOM | 50 | C5 | MAN | 7 | 5.287 | 28.108 | 25.140 | −0.15 | +0.09 | +0.113 | 3.664 |
| ATOM | 51 | C6 | MAN | 7 | 5.473 | 28.817 | 23.814 | −0.06 | +0.09 | +0.113 | 3.664 |
| ATOM | 52 | O6 | MAN | 7 | 4.277 | 29.461 | 23.400 | +0.22 | −0.40 | −0.537 | 3.664 |
| ATOM | 53 | H6 | MAN | 7 | 3.467 | 28.878 | 23.655 | +0.11 | +0.34 | +0.424 | 3.664 |
| ATOM | 54 | C4 | MAN | 7 | 6.589 | 28.087 | 25.950 | −0.16 | +0.06 | +0.113 | 3.664 |
| ATOM | 55 | O4 | MAN | 7 | 6.860 | 29.404 | 26.399 | +0.18 | −0.25 | −0.537 | 3.664 |
| ATOM | 56 | HO4 | MAN | 7 | 7.225 | 29.371 | 27.361 | +0.09 | +0.13 | +0.424 | 3.664 |
| ATOM | 57 | C3 | MAN | 7 | 6.500 | 27.147 | 27.158 | −0.36 | +0.04 | +0.113 | 3.664 |
| ATOM | 58 | O3 | MAN | 7 | 7.790 | 26.985 | 27.730 | +0.04 | −0.12 | −0.537 | 3.664 |
| ATOM | 59 | HO3 | MAN | 7 | 7.737 | 27.143 | 28.747 | −0.09 | −0.34 | +0.424 | 3.664 |
| ATOM | 60 | C2 | MAN | 7 | 5.940 | 25.776 | 26.764 | −0.51 | +0.07 | +0.113 | 3.664 |
| ATOM | 61 | O2 | MAN | 7 | 6.879 | 25.082 | 25.957 | −0.03 | −0.35 | −0.537 | 3.664 |
| ATOM | 62 | HO2 | MAN | 7 | 7.832 | 25.396 | 26.191 | +0.08 | +0.26 | +0.424 | 3.664 |
| ATOM | 63 | C4 | MAN | 7 | 0.953 | 28.251 | 26.609 | −0.27 | +0.10 | +0.113 | 3.664 |
| ATOM | 64 | O4 | MAN | 7 | 1.924 | 28.847 | 27.456 | +0.12 | −0.37 | −0.537 | 3.664 |
| ATOM | 65 | HO4 | MAN | 7 | 1.891 | 28.402 | 28.385 | +0.09 | +0.29 | +0.424 | 3.664 |
| ATOM | 66 | C3 | MAN | 7 | 0.366 | 29.295 | 25.656 | −0.13 | +0.09 | +0.113 | 3.664 |
| ATOM | 67 | O3 | MAN | 7 | −0.155 | 30.463 | 26.350 | +0.21 | −0.17 | −0.227 | 3.664 |
| ATOM | 68 | C1 | BDG | 7 | −0.495 | 31.604 | 25.560 | −0.04 | +0.17 | +0.227 | 3.664 |
| ATOM | 69 | C2 | BDG | 7 | −0.294 | 32.893 | 26.389 | −0.02 | +0.07 | +0.113 | 3.664 |
| ATOM | 70 | O22 | BDG | 7 | −0.485 | 34.025 | 25.537 | +0.23 | −0.07 | −0.113 | 3.664 |
| ATOM | 71 | C3 | BDG | 7 | −1.313 | 32.973 | 27.550 | −0.03 | +0.07 | +0.113 | 3.664 |
| ATOM | 72 | O3 | BDG | 7 | −1.209 | 34.219 | 28.192 | +0.23 | −0.30 | −0.537 | 3.664 |
| ATOM | 73 | HO3 | BDG | 7 | −1.844 | 34.214 | 28.932 | +0.12 | +0.23 | +0.424 | 3.664 |
| ATOM | 74 | C4 | BDG | 7 | −2.772 | 32.753 | 27.072 | −0.04 | +0.08 | +0.113 | 3.664 |
| ATOM | 75 | O4 | BDG | 7 | −3.621 | 32.670 | 28.193 | +0.22 | −0.36 | −0.537 | 3.664 |
| ATOM | 76 | HO4 | BDG | 7 | −4.528 | 32.558 | 27.857 | +0.12 | +0.31 | +0.424 | 3.664 |
| ATOM | 77 | C5 | BDG | 7 | −2.881 | 31.469 | 26.196 | −0.12 | +0.10 | +0.113 | 3.664 |
| ATOM | 78 | C6 | BDG | 7 | −4.273 | 31.287 | 25.524 | −0.09 | +0.12 | +0.113 | 3.664 |
| ATOM | 79 | O6 | BDG | 7 | −5.280 | 31.054 | 26.485 | +0.09 | −0.54 | −0.537 | 3.664 |
| ATOM | 80 | H6 | BDG | 7 | −6.101 | 30.943 | 26.002 | +0.08 | +0.48 | +0.424 | 3.664 |
| ATOM | 81 | O | BDG | 7 | −1.886 | 31.497 | 25.141 | +0.19 | −0.20 | −0.227 | 3.664 |
| ATOM | 82 | C2 | MAN | 7 | −0.699 | 28.637 | 24.766 | −0.24 | +0.11 | +0.113 | 3.664 |
| ATOM | 83 | O2 | MAN | 7 | −1.766 | 28.161 | 25.573 | −0.01 | −0.63 | −0.537 | 3.664 |
| ATOM | 84 | HO2 | MAN | 7 | −2.580 | 27.948 | 24.978 | +0.07 | +0.73 | +0.424 | 3.664 |
| ATOM | 85 | C4 | MAN | 7 | −1.939 | 29.238 | 20.412 | −0.41 | +0.12 | +0.113 | 3.664 |
| ATOM | 86 | O4 | MAN | 7 | −2.940 | 28.383 | 20.947 | −0.09 | −0.62 | −0.537 | 3.664 |
| ATOM | 87 | HO4 | MAN | 7 | −2.593 | 27.946 | 21.812 | +0.07 | +0.54 | +0.424 | 3.664 |
| TER | | | | | | | | | | | |
| ENDMDL | | | | | | | | | | | |
| MODEL | 32 | | | | | | | | | | |
| USER | Run = 32 | | | | | | | | | | |
| USER | Cluster Rank = 8 | | | | | | | | | | |
| USER | Number of conformations in this cluster = 1 | | | | | | | | | | |
| USER | | | | | | | | | | | |
| USER | RMSD from reference structure = 4.670 A | | | | | | | | | | |
| USER | | | | | | | | | | | |
| USER | Estimated Free Energy of Binding = +0.55 kcal/mol [= (1) + (3)] | | | | | | | | | | |
| USER | Estimated Inhibition Constant, Ki = +2.54 [Temperature = 298.15 K] | | | | | | | | | | |
| USER | | | | | | | | | | | |
| USER | Final Docked Energy = −2.39 kcal/mol [= (1) + (2)] | | | | | | | | | | |
| USER | | | | | | | | | | | |
| USER | (1) Final Intermolecular Energy = −4.43 kcal/mol | | | | | | | | | | |
| USER | (2) Final Internal Energy of Ligand = +2.04 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 −1.744661 30.964082 22.434648 | | | | | | | | | | |
| USER | NEWDPF quat0 0.765741 0.619767 0.171843 −112.841517 | | | | | | | | | | |
| USER | NEWDPF ndihe 16 | | | | | | | | | | |
| USER | NEWDPF dihe0 −99.04 −56.54 −127.16 −69.61 22.73 86.40 167.00 −17.99 8.60 | | | | | | | | | | |
| | 6.72 147.07 124.33 121.60 48.35 25.36 −66.80 | | | | | | | | | | |
| USER | | | | | | | | | | | |
| USER | | | | Rank | x | y | z | vdW | Elec | q | RMS |
| ATOM | 1 | C5 | MAN | 8 | −3.832 | 30.441 | 29.057 | −0.20 | +0.09 | +0.113 | 4.670 |
| ATOM | 2 | HO4 | MAN | 8 | −3.287 | 32.901 | 28.153 | +0.12 | +0.28 | +0.424 | 4.670 |
| ATOM | 3 | O4 | MAN | 8 | −4.091 | 32.814 | 28.792 | +0.22 | −0.35 | −0.537 | 4.670 |
| ATOM | 4 | O5 | MAN | 8 | −4.329 | 29.158 | 28.612 | +0.04 | −0.24 | −0.227 | 4.670 |
| ATOM | 5 | HO2 | MAN | 8 | −7.268 | 29.061 | 26.835 | +0.06 | +0.66 | +0.424 | 4.670 |
| ATOM | 6 | O2 | MAN | 8 | −6.764 | 29.796 | 27.350 | −0.02 | −0.63 | −0.537 | 4.670 |
| ATOM | 7 | C2 | MAN | 8 | −5.523 | 30.048 | 26.720 | −0.16 | +0.13 | +0.113 | 4.670 |
| ATOM | 8 | C1 | MAN | 8 | −4.490 | 29.042 | 27.196 | −0.23 | +0.26 | +0.227 | 4.670 |
| ATOM | 9 | C4 | MAN | 8 | −4.743 | 31.577 | 28.565 | −0.11 | +0.09 | +0.113 | 4.670 |
| ATOM | 10 | O3 | MAN | 8 | −6.041 | 32.391 | 26.696 | +0.18 | −0.45 | −0.537 | 4.670 |
| ATOM | 11 | HO3 | MAN | 8 | −6.162 | 32.381 | 25.673 | +0.11 | +0.40 | +0.424 | 4.670 |
| ATOM | 12 | C3 | MAN | 8 | −5.046 | 31.451 | 27.064 | −0.17 | +0.10 | +0.113 | 4.670 |
| ATOM | 13 | C6 | MAN | 8 | −3.751 | 30.387 | 30.574 | −0.25 | +0.09 | +0.113 | 4.670 |
| ATOM | 14 | O6 | MAN | 8 | −4.234 | 29.146 | 31.076 | +0.17 | −0.47 | −0.537 | 4.670 |
| ATOM | 15 | H6 | MAN | 8 | −4.823 | 28.694 | 30.364 | +0.09 | +0.42 | +0.424 | 4.670 |
| ATOM | 16 | O3 | MAN | 8 | −3.300 | 29.288 | 26.516 | +0.04 | −0.26 | −0.227 | 4.670 |
| ATOM | 17 | C3 | MAN | 8 | −2.313 | 28.259 | 26.615 | −0.42 | +0.14 | +0.113 | 4.670 |
| ATOM | 18 | C2 | MAN | 8 | −1.187 | 28.781 | 27.501 | −0.31 | +0.12 | +0.113 | 4.670 |
| ATOM | 19 | O2 | MAN | 8 | −0.790 | 30.077 | 27.071 | +0.19 | −0.43 | −0.537 | 4.670 |
| ATOM | 20 | HO2 | MAN | 8 | 0.211 | 30.214 | 27.273 | +0.11 | +0.30 | +0.424 | 4.670 |
| ATOM | 21 | C1 | MAN | 8 | −0.003 | 27.843 | 27.445 | −0.39 | +0.26 | +0.227 | 4.670 |
| ATOM | 22 | O4 | NAG | 8 | 1.037 | 28.372 | 28.185 | +0.01 | −0.19 | −0.227 | 4.670 |
| ATOM | 23 | C4 | NAG | 8 | 2.250 | 28.691 | 27.491 | −0.29 | +0.08 | +0.113 | 4.670 |
| ATOM | 24 | C5 | NAG | 8 | 3.261 | 27.547 | 27.716 | −0.46 | +0.06 | +0.113 | 4.670 |
| ATOM | 25 | C6 | NAG | 8 | 2.980 | 26.301 | 26.892 | −0.03 | +0.08 | +0.113 | 4.670 |
| ATOM | 26 | O6 | NAG | 8 | 3.999 | 26.064 | 25.928 | −0.13 | −0.56 | −0.537 | 4.670 |
| ATOM | 27 | H6 | NAG | 8 | 4.891 | 26.449 | 26.270 | +0.07 | +0.31 | +0.424 | 4.670 |
| ATOM | 28 | O5 | NAG | 8 | 4.568 | 27.965 | 27.350 | +0.04 | −0.10 | −0.227 | 4.670 |
| ATOM | 29 | C1 | NAG | 8 | 5.019 | 29.054 | 28.130 | +0.14 | +0.04 | +0.113 | 4.670 |
| ATOM | 30 | C2 | NAG | 8 | 4.211 | 30.289 | 27.697 | −0.20 | +0.10 | +0.211 | 4.670 |
| ATOM | 31 | N2 | NAG | 8 | 4.650 | 31.480 | 28.389 | −0.12 | −0.28 | −0.650 | 4.670 |
| ATOM | 32 | HN2 | NAG | 8 | 4.377 | 31.633 | 29.369 | +0.09 | +0.15 | +0.440 | 4.670 |
| ATOM | 33 | C7 | NAG | 8 | 5.407 | 32.380 | 27.764 | −0.12 | +0.19 | +0.396 | 4.670 |
| ATOM | 34 | O7 | NAG | 8 | 5.766 | 32.255 | 26.595 | +0.21 | −0.20 | −0.396 | 4.670 |
| ATOM | 35 | C8 | NAG | 8 | 5.835 | 33.601 | 28.557 | −0.09 | +0.00 | +0.000 | 4.670 |
| ATOM | 36 | C3 | NAG | 8 | 2.744 | 30.038 | 28.023 | −0.16 | +0.06 | +0.113 | 4.670 |
| ATOM | 37 | O3 | NAG | 8 | 1.943 | 31.073 | 27.471 | +0.18 | −0.30 | −0.537 | 4.670 |
| ATOM | 38 | HO3 | NAG | 8 | 1.912 | 30.975 | 26.447 | +0.12 | +0.26 | +0.424 | 4.670 |
| ATOM | 39 | O5 | MAN | 8 | 0.417 | 27.673 | 26.083 | +0.08 | −0.22 | −0.227 | 4.670 |
| ATOM | 40 | C5 | MAN | 8 | −0.575 | 26.981 | 25.317 | −0.48 | +0.11 | +0.113 | 4.670 |
| ATOM | 41 | C6 | MAN | 8 | 0.081 | 26.478 | 24.024 | −0.26 | +0.08 | +0.113 | 4.670 |
| ATOM | 42 | O6 | MAN | 8 | 1.511 | 26.727 | 24.011 | −0.02 | −0.22 | −0.227 | 4.670 |
| ATOM | 43 | C1 | MAN | 8 | 1.916 | 27.871 | 23.287 | −0.24 | +0.19 | +0.227 | 4.670 |
| ATOM | 44 | O5 | MAN | 8 | 2.964 | 28.637 | 23.908 | +0.18 | −0.19 | −0.227 | 4.670 |
| ATOM | 45 | C5 | MAN | 8 | 4.288 | 28.046 | 23.854 | −0.14 | +0.11 | +0.113 | 4.670 |
| ATOM | 46 | C6 | MAN | 8 | 5.250 | 28.939 | 24.633 | −0.08 | +0.08 | +0.113 | 4.670 |
| ATOM | 47 | O6 | MAN | 8 | 6.284 | 28.148 | 25.253 | +0.19 | −0.15 | −0.227 | 4.670 |
| ATOM | 48 | C1 | MAN | 8 | 7.517 | 28.818 | 25.328 | −0.07 | +0.13 | +0.227 | 4.670 |
| ATOM | 49 | O5 | MAN | 8 | 8.273 | 28.452 | 26.485 | +0.17 | −0.10 | −0.227 | 4.670 |
| ATOM | 50 | C5 | MAN | 8 | 8.597 | 27.044 | 26.569 | −0.23 | +0.06 | +0.113 | 4.670 |
| ATOM | 51 | C6 | MAN | 8 | 9.399 | 26.842 | 27.838 | −0.34 | +0.07 | +0.113 | 4.670 |
| ATOM | 52 | O6 | MAN | 8 | 8.724 | 25.980 | 28.744 | −0.22 | −0.38 | −0.537 | 4.670 |
| ATOM | 53 | H6 | MAN | 8 | 7.711 | 26.163 | 28.703 | −0.09 | +0.09 | +0.424 | 4.670 |
| ATOM | 54 | C4 | MAN | 8 | 9.382 | 26.588 | 25.334 | −0.14 | +0.07 | +0.113 | 4.670 |
| ATOM | 55 | O4 | MAN | 8 | 9.474 | 25.174 | 25.357 | +0.11 | −0.33 | −0.537 | 4.670 |
| ATOM | 56 | HO4 | MAN | 8 | 9.357 | 24.806 | 24.402 | +0.10 | +0.26 | +0.424 | 4.670 |
| ATOM | 57 | C3 | MAN | 8 | 8.710 | 27.031 | 24.029 | −0.07 | +0.08 | +0.113 | 4.670 |
| ATOM | 58 | O3 | MAN | 8 | 9.587 | 26.793 | 22.937 | +0.22 | −0.37 | −0.537 | 4.670 |
| ATOM | 59 | HO3 | MAN | 8 | 9.082 | 26.288 | 22.194 | +0.12 | +0.31 | +0.424 | 4.670 |
| ATOM | 60 | C2 | MAN | 8 | 8.333 | 28.516 | 24.066 | −0.03 | +0.07 | +0.113 | 4.670 |
| ATOM | 61 | O2 | MAN | 8 | 9.507 | 29.316 | 24.045 | +0.23 | −0.32 | −0.537 | 4.670 |
| ATOM | 62 | HO2 | MAN | 8 | 10.260 | 28.807 | 23.559 | +0.12 | +0.26 | +0.424 | 4.670 |
| ATOM | 63 | C4 | MAN | 8 | 4.706 | 27.816 | 22.399 | −0.13 | +0.11 | +0.113 | 4.670 |
| ATOM | 64 | O4 | MAN | 8 | 6.005 | 27.244 | 22.350 | +0.19 | −0.54 | −0.537 | 4.670 |
| ATOM | 65 | HO4 | MAN | 8 | 6.702 | 27.986 | 22.188 | +0.12 | +0.35 | +0.424 | 4.670 |
| ATOM | 66 | C3 | MAN | 8 | 3.679 | 26.853 | 21.796 | −0.11 | +0.13 | +0.113 | 4.670 |
| ATOM | 67 | O3 | MAN | 8 | 4.036 | 26.414 | 20.456 | +0.11 | −0.23 | −0.227 | 4.670 |
| ATOM | 68 | C1 | BDG | 8 | 4.394 | 27.419 | 19.505 | −0.19 | +0.20 | +0.227 | 4.670 |
| ATOM | 69 | C2 | BDG | 8 | 3.537 | 27.260 | 18.228 | −0.34 | +0.10 | +0.113 | 4.670 |
| ATOM | 70 | O22 | BDG | 8 | 3.797 | 28.363 | 17.357 | +0.00 | −0.10 | −0.113 | 4.670 |
| ATOM | 71 | C3 | BDG | 8 | 3.888 | 25.950 | 17.482 | −0.42 | +0.11 | +0.113 | 4.670 |
| ATOM | 72 | O3 | BDG | 8 | 3.208 | 25.905 | 16.253 | +0.21 | −0.60 | −0.537 | 4.670 |
| ATOM | 73 | HO3 | BDG | 8 | 3.433 | 25.054 | 15.833 | +0.18 | +0.56 | +0.424 | 4.670 |
| ATOM | 74 | C4 | BDG | 8 | 5.412 | 25.798 | 17.241 | −0.33 | +0.13 | +0.113 | 4.670 |
| ATOM | 75 | O4 | BDG | 8 | 5.674 | 24.518 | 16.714 | −0.20 | −0.84 | −0.537 | 4.670 |
| ATOM | 76 | HO4 | BDG | 8 | 6.633 | 24.465 | 16.552 | +0.08 | +0.61 | +0.424 | 4.670 |
| ATOM | 77 | C5 | BDG | 8 | 6.209 | 26.030 | 18.560 | −0.14 | +0.11 | +0.113 | 4.670 |
| ATOM | 78 | C6 | BDG | 8 | 7.751 | 26.081 | 18.358 | −0.12 | +0.11 | +0.113 | 4.670 |
| ATOM | 79 | O6 | BDG | 8 | 8.256 | 24.830 | 17.945 | +0.15 | −0.54 | −0.537 | 4.670 |
| ATOM | 80 | H6 | BDG | 8 | 9.205 | 24.934 | 17.850 | +0.11 | +0.43 | +0.424 | 4.670 |
| ATOM | 81 | O | BDG | 8 | 5.805 | 27.280 | 19.174 | +0.19 | −0.21 | −0.227 | 4.670 |
| ATOM | 82 | C2 | MAN | 8 | 2.286 | 27.498 | 21.835 | −0.20 | +0.08 | +0.113 | 4.670 |
| ATOM | 83 | O2 | MAN | 8 | 2.281 | 28.670 | 21.032 | +0.14 | −0.40 | −0.537 | 4.670 |
| ATOM | 84 | HO2 | MAN | 8 | 1.310 | 28.963 | 20.855 | +0.10 | +0.31 | +0.424 | 4.670 |
| ATOM | 85 | C4 | MAN | 8 | −1.823 | 27.867 | 25.200 | −0.43 | +0.14 | +0.113 | 4.670 |
| ATOM | 86 | O4 | MAN | 8 | −2.849 | 27.156 | 24.522 | −0.13 | −1.03 | −0.537 | 4.670 |
| ATOM | 87 | HO4 | MAN | 8 | −2.497 | 26.230 | 24.239 | −0.01 | +0.40 | +0.424 | 4.670 |
| TER | | | | | | | | | | | |
| ENDMDL | | | | | | | | | | | |
| MODEL | 28 | | | | | | | | | | |
| USER | Run = 28 | | | | | | | | | | |
| USER | Cluster Rank = 9 | | | | | | | | | | |
| USER | Number of conformations in this cluster = 1 | | | | | | | | | | |
| USER | | | | | | | | | | | |
| USER | RMSD from reference structure = 3.635 A | | | | | | | | | | |
| USER | | | | | | | | | | | |
| USER | Estimated Free Energy of Binding = −0.43 kcal/mol [= (1) + (3)] | | | | | | | | | | |
| USER | Estimated Inhibition Constant, Ki = +0.48 [Temperature = 298.15 K] | | | | | | | | | | |
| USER | | | | | | | | | | | |
| USER | Final Docked Energy = −2.23 kcal/mol [= (1) + (2)] | | | | | | | | | | |
| USER | | | | | | | | | | | |
| USER | (1) Final Intermolecular Energy = −5.41 kcal/mol | | | | | | | | | | |
| USER | (2) Final Internal Energy of Ligand = +3.18 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 −3.637806 31.531266 22.281049 | | | | | | | | | | |
| USER | NEWDPF quat0 −0.590159 −0.783732 0.193590 50.454746 | | | | | | | | | | |
| USER | NEWDPF ndihe 16 | | | | | | | | | | |
| USER | NEWDPF dihe0 179.59 178.97 −106.37 −104.04 13.20 12.48 −43.63 −25.81 | | | | | | | | | | |
| | 106.01 −69.71 −173.93 28.93 65.52 10.79 67.39 −35.49 | | | | | | | | | | |
| USER | | | | | | | | | | | |
| USER | | | | Rank | x | y | z | vdW | Elec | q | RMS |
| ATOM | 1 | C5 | MAN | 9 | 0.442 | 27.268 | 25.979 | −0.40 | +0.11 | +0.113 | 3.635 |
| ATOM | 2 | HO4 | MAN | 9 | −0.360 | 29.520 | 27.184 | +0.11 | +0.35 | +0.424 | 3.635 |
| ATOM | 3 | O4 | MAN | 9 | −0.316 | 28.675 | 27.772 | +0.08 | −0.51 | −0.537 | 3.635 |
| ATOM | 4 | O5 | MAN | 9 | 0.009 | 26.296 | 24.999 | −0.08 | −0.20 | −0.227 | 3.635 |
| ATOM | 5 | HO2 | MAN | 9 | −3.114 | 24.904 | 25.330 | −0.26 | +0.09 | +0.424 | 3.635 |
| ATOM | 6 | O2 | MAN | 9 | −2.531 | 25.512 | 25.923 | −0.23 | −0.38 | −0.537 | 3.635 |
| ATOM | 7 | C2 | MAN | 9 | −2.337 | 26.755 | 25.277 | −0.58 | +0.14 | +0.113 | 3.635 |
| ATOM | 8 | C1 | MAN | 9 | −1.183 | 26.651 | 24.296 | −0.43 | +0.20 | +0.227 | 3.635 |
| ATOM | 9 | C4 | MAN | 9 | −0.676 | 27.543 | 26.999 | −0.43 | +0.13 | +0.113 | 3.635 |
| ATOM | 10 | O3 | MAN | 9 | −3.053 | 27.875 | 27.275 | −0.06 | −0.77 | −0.537 | 3.635 |
| ATOM | 11 | HO3 | MAN | 9 | −3.930 | 28.174 | 26.825 | +0.06 | +0.57 | +0.424 | 3.635 |
| ATOM | 12 | C3 | MAN | 9 | −2.018 | 27.827 | 26.309 | −0.46 | +0.14 | +0.113 | 3.635 |
| ATOM | 13 | C6 | MAN | 9 | 1.695 | 26.714 | 26.636 | −0.50 | +0.12 | +0.113 | 3.635 |
| ATOM | 14 | O6 | MAN | 9 | 1.576 | 26.699 | 28.053 | −0.36 | −0.75 | −0.537 | 3.635 |
| ATOM | 15 | H6 | MAN | 9 | 2.084 | 27.502 | 28.448 | +0.07 | +0.34 | +0.424 | 3.635 |
| ATOM | 16 | O3 | MAN | 9 | −1.057 | 27.875 | 23.644 | +0.01 | −0.23 | −0.227 | 3.635 |
| ATOM | 17 | C3 | MAN | 9 | −0.870 | 27.798 | 22.228 | −0.41 | +0.09 | +0.113 | 3.635 |
| ATOM | 18 | C2 | MAN | 9 | 0.492 | 27.161 | 21.975 | −0.04 | +0.05 | +0.113 | 3.635 |
| ATOM | 19 | O2 | MAN | 9 | 1.484 | 27.801 | 22.768 | +0.09 | −0.40 | −0.537 | 3.635 |
| ATOM | 20 | HO2 | MAN | 9 | 2.389 | 27.768 | 22.278 | +0.09 | +0.35 | +0.424 | 3.635 |
| ATOM | 21 | C1 | MAN | 9 | 0.858 | 27.278 | 20.514 | −0.37 | +0.11 | +0.227 | 3.635 |
| ATOM | 22 | O4 | NAG | 9 | 2.128 | 26.772 | 20.317 | +0.05 | −0.11 | −0.227 | 3.635 |
| ATOM | 23 | C4 | NAG | 9 | 2.536 | 26.506 | 18.969 | −0.37 | +0.08 | +0.113 | 3.635 |
| ATOM | 24 | C5 | NAG | 9 | 2.562 | 24.978 | 18.755 | −0.46 | +0.08 | +0.113 | 3.635 |
| ATOM | 25 | C6 | NAG | 9 | 1.188 | 24.352 | 18.585 | −0.64 | +0.05 | +0.113 | 3.635 |
| ATOM | 26 | O6 | NAG | 9 | 1.005 | 23.823 | 17.278 | −0.18 | −0.34 | −0.537 | 3.635 |
| ATOM | 27 | H6 | NAG | 9 | 1.917 | 23.540 | 16.892 | +0.07 | +0.32 | +0.424 | 3.635 |
| ATOM | 28 | O5 | NAG | 9 | 3.280 | 24.659 | 17.570 | −0.01 | −0.21 | −0.227 | 3.635 |
| ATOM | 29 | C1 | NAG | 9 | 4.629 | 25.076 | 17.634 | −0.34 | +0.12 | +0.113 | 3.635 |
| ATOM | 30 | C2 | NAG | 9 | 4.635 | 26.610 | 17.546 | −0.36 | +0.21 | +0.211 | 3.635 |
| ATOM | 31 | N2 | NAG | 9 | 5.982 | 27.136 | 17.529 | −0.14 | −0.66 | −0.650 | 3.635 |
| ATOM | 32 | HN2 | NAG | 9 | 6.337 | 27.663 | 18.338 | +0.11 | +0.41 | +0.440 | 3.635 |
| ATOM | 33 | C7 | NAG | 9 | 6.765 | 26.942 | 16.471 | −0.33 | +0.47 | +0.396 | 3.635 |
| ATOM | 34 | O7 | NAG | 9 | 6.401 | 26.321 | 15.473 | +0.13 | −0.56 | −0.396 | 3.635 |
| ATOM | 35 | C8 | NAG | 9 | 8.169 | 27.517 | 16.528 | −0.24 | +0.00 | +0.000 | 3.635 |
| ATOM | 36 | C3 | NAG | 9 | 3.902 | 27.162 | 18.763 | −0.29 | +0.10 | +0.113 | 3.635 |
| ATOM | 37 | O3 | NAG | 9 | 3.725 | 28.564 | 18.625 | +0.07 | −0.44 | −0.537 | 3.635 |
| ATOM | 38 | HO3 | NAG | 9 | 3.019 | 28.752 | 17.900 | +0.07 | +0.33 | +0.424 | 3.635 |
| ATOM | 39 | O5 | MAN | 9 | 0.824 | 28.656 | 20.114 | +0.01 | −0.16 | −0.227 | 3.635 |
| ATOM | 40 | C5 | MAN | 9 | −0.509 | 29.175 | 20.151 | −0.35 | +0.09 | +0.113 | 3.635 |
| ATOM | 41 | C6 | MAN | 9 | −0.539 | 30.480 | 19.344 | −0.23 | +0.12 | +0.113 | 3.635 |
| ATOM | 42 | O6 | MAN | 9 | 0.722 | 31.192 | 19.420 | +0.02 | −0.24 | −0.227 | 3.635 |
| ATOM | 43 | C1 | MAN | 9 | 0.640 | 32.587 | 19.207 | −0.06 | +0.33 | +0.227 | 3.635 |
| ATOM | 44 | O5 | MAN | 9 | −0.446 | 33.238 | 19.890 | −0.07 | −0.25 | −0.227 | 3.635 |
| ATOM | 45 | C5 | MAN | 9 | −0.282 | 33.394 | 21.323 | −0.10 | +0.09 | +0.113 | 3.635 |
| ATOM | 46 | C6 | MAN | 9 | −1.554 | 34.014 | 21.893 | −0.07 | +0.08 | +0.113 | 3.635 |
| ATOM | 47 | O6 | MAN | 9 | −1.381 | 34.333 | 23.288 | +0.23 | −0.15 | −0.227 | 3.635 |
| ATOM | 48 | C1 | MAN | 9 | −2.225 | 33.591 | 24.130 | −0.04 | +0.17 | +0.227 | 3.635 |
| ATOM | 49 | O5 | MAN | 9 | −1.814 | 32.228 | 24.260 | +0.20 | −0.19 | −0.227 | 3.635 |
| ATOM | 50 | C5 | MAN | 9 | −0.482 | 32.052 | 24.801 | −0.03 | +0.08 | +0.113 | 3.635 |
| ATOM | 51 | C6 | MAN | 9 | −0.220 | 30.562 | 24.875 | −0.07 | +0.09 | +0.113 | 3.635 |
| ATOM | 52 | O6 | MAN | 9 | 1.102 | 30.248 | 24.462 | +0.22 | −0.40 | −0.537 | 3.635 |
| ATOM | 53 | H6 | MAN | 9 | 1.372 | 30.861 | 23.679 | +0.12 | +0.31 | +0.424 | 3.635 |
| ATOM | 54 | C4 | MAN | 9 | −0.360 | 32.711 | 26.179 | −0.02 | +0.07 | +0.113 | 3.635 |
| ATOM | 55 | O4 | MAN | 9 | 1.005 | 32.709 | 26.559 | +0.23 | −0.32 | −0.537 | 3.635 |
| ATOM | 56 | HO4 | MAN | 9 | 1.234 | 33.598 | 27.024 | +0.12 | +0.23 | +0.424 | 3.635 |
| ATOM | 57 | C3 | MAN | 9 | −0.885 | 34.151 | 26.178 | −0.01 | +0.07 | +0.113 | 3.635 |
| ATOM | 58 | O3 | MAN | 9 | −0.967 | 34.629 | 27.512 | +0.23 | −0.30 | −0.537 | 3.635 |
| ATOM | 59 | HO3 | MAN | 9 | −0.507 | 35.549 | 27.577 | +0.12 | +0.22 | +0.424 | 3.635 |
| ATOM | 60 | C2 | MAN | 9 | −2.262 | 34.248 | 25.514 | −0.02 | +0.07 | +0.113 | 3.635 |
| ATOM | 61 | O2 | MAN | 9 | −3.238 | 33.613 | 26.327 | +0.22 | −0.37 | −0.537 | 3.635 |
| ATOM | 62 | HO2 | MAN | 9 | −2.943 | 33.654 | 27.314 | +0.12 | +0.27 | +0.424 | 3.635 |
| ATOM | 63 | C4 | MAN | 9 | 0.984 | 34.202 | 21.625 | −0.07 | +0.07 | +0.113 | 3.635 |
| ATOM | 64 | O4 | MAN | 9 | 1.137 | 34.359 | 23.028 | +0.23 | −0.33 | −0.537 | 3.635 |
| ATOM | 65 | HO4 | MAN | 9 | 0.765 | 35.276 | 23.315 | +0.12 | +0.24 | +0.4$$4 | 3.635 |
| ATOM | 66 | C3 | MAN | 9 | 2.159 | 33.408 | 21.049 | −0.08 | +0.08 | +0.1$$3 | 3.635 |
| ATOM | 67 | O3 | MAN | 9 | 3.451 | 33.976 | 21.399 | +0.22 | −0.14 | −0.227 | 3.635 |
| ATOM | 68 | C1 | BDG | 9 | 4.165 | 33.376 | 22.481 | +0.00 | +0.14 | +0.227 | 3.635 |
| ATOM | 69 | C2 | BDG | 9 | 4.758 | 34.477 | 23.391 | +0.00 | +0.06 | +0.113 | 3.635 |
| ATOM | 70 | O22 | BDG | 9 | 5.330 | 33.862 | 24.548 | +0.23 | −0.06 | −0.113 | 3.635 |
| ATOM | 71 | C3 | BDG | 9 | 5.864 | 35.271 | 22.657 | +0.00 | +0.06 | +0.113 | 3.635 |
| ATOM | 72 | O3 | BDG | 9 | 6.487 | 36.159 | 23.550 | +0.24 | −0.28 | −0.537 | 3.635 |
| ATOM | 73 | HO3 | BDG | 9 | 7.153 | 36.656 | 23.040 | +0.12 | +0.22 | +0.424 | 3.635 |
| ATOM | 74 | C4 | BDG | 9 | 6.929 | 34.344 | 22.015 | +0.00 | +0.07 | +0.113 | 3.635 |
| ATOM | 75 | O4 | BDG | 9 | 7.804 | 35.116 | 21.227 | +0.23 | −0.32 | −0.537 | 3.635 |
| ATOM | 76 | HO4 | BDG | 9 | 8.472 | 34.509 | 20.861 | +0.12 | +0.27 | +0.424 | 3.635 |
| ATOM | 77 | C5 | BDG | 9 | 6.253 | 33.227 | 21.164 | −0.01 | +0.07 | +0.113 | 3.635 |
| ATOM | 78 | C6 | BDG | 9 | 7.246 | 32.148 | 20.645 | −0.02 | +0.08 | +0.113 | 3.635 |
| ATOM | 79 | O6 | BDG | 9 | 8.157 | 32.694 | 19.715 | +0.22 | −0.40 | −0.537 | 3.635 |
| ATOM | 80 | H6 | BDG | 9 | 8.727 | 31.975 | 19.434 | +0.12 | +0.33 | +0.424 | 3.635 |
| ATOM | 81 | O | BDG | 9 | 5.237 | 32.549 | 21.945 | +0.23 | −0.15 | −0.227 | 3.635 |
| ATOM | 82 | C2 | MAN | 9 | 1.988 | 33.270 | 19.528 | −0.23 | +0.10 | +0.113 | 3.635 |
| ATOM | 83 | O2 | MAN | 9 | 2.019 | 34.557 | 18.928 | +0.03 | −0.38 | −0.537 | 3.635 |
| ATOM | 84 | HO2 | MAN | 9 | 2.153 | 34.459 | 17.911 | +0.06 | +0.34 | +0.424 | 3.635 |
| ATOM | 85 | C4 | MAN | 9 | −0.989 | 29.212 | 21.608 | −0.29 | +0.11 | +0.113 | 3.635 |
| ATOM | 86 | O4 | MAN | 9 | −2.344 | 29.637 | 21.652 | +0.02 | −0.80 | −0.537 | 3.635 |
| ATOM | 87 | HO4 | MAN | 9 | −2.667 | 29.854 | 20.699 | +0.09 | +0.52 | +0.424 | 3.635 |
| TER | | | | | | | | | | | |
| ENDMDL | | | | | | | | | | | |
| MODEL | 82 | | | | | | | | | | |
| USER | Run = 82 | | | | | | | | | | |
| USER | Cluster Rank = 10 | | | | | | | | | | |
| USER | Number of conformations in this cluster = 1 | | | | | | | | | | |
| USER | | | | | | | | | | | |
| USER | RMSD from reference structure = 3.976 A | | | | | | | | | | |
| USER | | | | | | | | | | | |
| USER | Estimated Free Energy of Binding = −3.82 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −2.08 kcal/mol [= (1) + (2)] | | | | | | | | | | |
| USER | | | | | | | | | | | |
| USER | (1) Final Intermolecular Energy = −8.80 kcal/mol | | | | | | | | | | |
| USER | (2) Final Internal Energy of Ligand = +6.72 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 3.160860 29.290699 21.244501 | | | | | | | | | | |
| USER | NEWDPF quat0 0.653117 −0.511031 −0.558826 39.979468 | | | | | | | | | | |
| USER | NEWDPF ndihe 16 | | | | | | | | | | |
| USER | NEWDPF dihe0 74.12 −103.33 5.49 −41.34 −5.08 95.14 11.09 152.81 | | | | | | | | | | |
| | −16.96 −16.83 −110.81 108.10 124.89 −4.74 −0.29 23.47 | | | | | | | | | | |
| USER | | | | | | | | | | | |
| USER | | | | Rank | x | y | z | vdW | Elec | q | RMS |
| ATOM | 1 | C5 | MAN | 10 | 5.749 | 23.764 | 17.891 | −0.26 | +0.12 | +0.113 | 3.976 |
| ATOM | 2 | HO4 | MAN | 10 | 5.882 | 23.727 | 20.564 | +0.10 | +0.54 | +0.424 | 3.976 |
| ATOM | 3 | O4 | MAN | 10 | 5.670 | 22.830 | 20.102 | +0.06 | −0.62 | −0.537 | 3.976 |
| ATOM | 4 | O5 | MAN | 10 | 4.931 | 24.263 | 16.807 | −0.22 | −0.33 | −0.227 | 3.976 |
| ATOM | 5 | HO2 | MAN | 10 | 1.552 | 23.647 | 16.880 | +0.06 | +0.31 | +0.424 | 3.976 |
| ATOM | 6 | O2 | MAN | 10 | 2.354 | 23.325 | 17.440 | −0.04 | −0.36 | −0.537 | 3.976 |
| ATOM | 7 | C2 | MAN | 10 | 2.923 | 24.423 | 18.125 | −0.45 | +0.09 | +0.113 | 3.976 |
| ATOM | 8 | C1 | MAN | 10 | 3.890 | 25.155 | 17.212 | −0.42 | +0.25 | +0.227 | 3.976 |
| ATOM | 9 | C4 | MAN | 10 | 4.880 | 23.068 | 18.951 | −0.26 | +0.11 | +0.113 | 3.976 |
| ATOM | 10 | O3 | MAN | 10 | 2.798 | 23.177 | 20.173 | −0.01 | −0.32 | −0.537 | 3.976 |
| ATOM | 11 | HO3 | MAN | 10 | 2.050 | 23.789 | 20.530 | +0.01 | −0.15 | +0.424 | 3.976 |
| ATOM | 12 | C3 | MAN | 10 | 3.675 | 23.931 | 19.353 | −0.35 | +0.10 | +0.113 | 3.976 |
| ATOM | 13 | C6 | MAN | 10 | 6.774 | 22.823 | 17.280 | −0.36 | +0.12 | +0.113 | 3.976 |
| ATOM | 14 | O6 | MAN | 10 | 6.159 | 21.878 | 16.413 | +0.10 | −0.49 | −0.537 | 3.976 |
| ATOM | 15 | H6 | MAN | 10 | 6.839 | 21.569 | 15.704 | +0.03 | +0.35 | +0.424 | 3.976 |
| ATOM | 16 | O3 | MAN | 10 | 4.388 | 26.257 | 17.901 | +0.06 | −0.22 | −0.227 | 3.976 |
| ATOM | 17 | C3 | MAN | 10 | 3.494 | 27.367 | 18.010 | −0.28 | +0.10 | +0.113 | 3.976 |
| ATOM | 18 | C2 | MAN | 10 | 4.282 | 28.631 | 17.680 | −0.37 | +0.10 | +0.113 | 3.976 |
| ATOM | 19 | O2 | MAN | 10 | 5.498 | 28.651 | 18.415 | +0.14 | −0.48 | −0.537 | 3.976 |
| ATOM | 20 | HO2 | MAN | 10 | 5.765 | 29.627 | 18.610 | +0.11 | +0.37 | +0.424 | 3.976 |
| ATOM | 21 | C1 | MAN | 10 | 3.464 | 29.857 | 18.017 | −0.38 | +0.19 | +0.227 | 3.976 |
| ATOM | 22 | O4 | NAG | 10 | 4.222 | 30.989 | 17.791 | +0.02 | −0.19 | −0.227 | 3.976 |
| ATOM | 23 | C4 | NAG | 10 | 4.093 | 32.071 | 18.721 | −0.23 | +0.09 | +0.113 | 3.976 |
| ATOM | 24 | C5 | NAG | 10 | 4.760 | 31.661 | 20.051 | −0.08 | +0.09 | +0.113 | 3.976 |
| ATOM | 25 | C6 | NAG | 10 | 6.279 | 31.712 | 20.024 | −0.05 | +0.09 | +0.113 | 3.976 |
| ATOM | 26 | O6 | NAG | 10 | 6.790 | 32.715 | 20.893 | +0.23 | −0.37 | −0.537 | 3.976 |
| ATOM | 27 | H6 | NAG | 10 | 6.132 | 32.861 | 21.672 | +0.12 | +0.28 | +0.424 | 3.976 |
| ATOM | 28 | O5 | NAG | 10 | 4.363 | 32.540 | 21.094 | +0.22 | −0.16 | −0.227 | 3.976 |
| ATOM | 29 | C1 | NAG | 10 | 2.968 | 32.504 | 21.320 | −0.05 | +0.08 | +0.113 | 3.976 |
| ATOM | 30 | C2 | NAG | 10 | 2.295 | 33.190 | 20.120 | −0.15 | +0.17 | +0.211 | 3.976 |
| ATOM | 31 | N2 | NAG | 10 | 0.862 | 33.272 | 20.291 | −0.11 | −0.57 | −0.650 | 3.976 |
| ATOM | 32 | HN2 | NAG | 10 | 0.332 | 32.440 | 20.583 | +0.11 | +0.41 | +0.440 | 3.976 |
| ATOM | 33 | C7 | NAG | 10 | 0.224 | 34.420 | 20.072 | −0.21 | +0.26 | +0.396 | 3.976 |
| ATOM | 34 | O7 | NAG | 10 | 0.799 | 35.449 | 19.721 | +0.11 | −0.11 | −0.396 | 3.976 |
| ATOM | 35 | C8 | NAG | 10 | −1.280 | 34.431 | 20.278 | −0.19 | +0.00 | +0.000 | 3.976 |
| ATOM | 36 | C3 | NAG | 10 | 2.601 | 32.375 | 18.870 | −0.29 | +0.11 | +0.113 | 3.976 |
| ATOM | 37 | O3 | NAG | 10 | 2.155 | 33.073 | 17.717 | −0.06 | −0.66 | −0.537 | 3.976 |
| ATOM | 38 | HO3 | NAG | 10 | 2.767 | 33.884 | 17.547 | +0.07 | +0.39 | +0.424 | 3.976 |
| ATOM | 39 | O5 | MAN | 10 | 3.069 | 29.812 | 19.396 | +0.11 | −0.18 | −0.227 | 3.976 |
| ATOM | 40 | C5 | MAN | 10 | 2.151 | 28.741 | 19.639 | −0.32 | +0.08 | +0.113 | 3.976 |
| ATOM | 41 | C6 | MAN | 10 | 1.467 | 28.991 | 20.989 | −0.22 | +0.08 | +0.113 | 3.976 |
| ATOM | 42 | O6 | MAN | 10 | 2.424 | 29.334 | 22.025 | +0.19 | −0.17 | −0.227 | 3.976 |
| ATOM | 43 | C1 | MAN | 10 | 2.853 | 28.254 | 22.829 | −0.17 | +0.20 | +0.227 | 3.976 |
| ATOM | 44 | O5 | MAN | 10 | 4.276 | 28.049 | 22.854 | +0.17 | −0.22 | −0.227 | 3.976 |
| ATOM | 45 | C5 | MAN | 10 | 5.035 | 29.017 | 23.624 | −0.06 | +0.09 | +0.113 | 3.976 |
| ATOM | 46 | C6 | MAN | 10 | 6.521 | 28.705 | 23.467 | −0.04 | +0.08 | +0.113 | 3.976 |
| ATOM | 47 | O6 | MAN | 10 | 7.066 | 28.199 | 24.702 | +0.21 | −0.15 | −0.227 | 3.976 |
| ATOM | 48 | C1 | MAN | 10 | 7.628 | 26.919 | 24.571 | −0.13 | +0.16 | +0.227 | 3.976 |
| ATOM | 49 | O5 | MAN | 10 | 8.608 | 26.642 | 25.575 | +0.16 | −0.14 | −0.227 | 3.976 |
| ATOM | 50 | C5 | MAN | 10 | 8.110 | 26.704 | 26.932 | −0.32 | +0.06 | +0.113 | 3.976 |
| ATOM | 51 | C6 | MAN | 10 | 9.269 | 26.379 | 27.852 | −0.40 | +0.08 | +0.113 | 3.976 |
| ATOM | 52 | O6 | MAN | 10 | 9.101 | 25.108 | 28.465 | −0.27 | −0.74 | −0.537 | 3.976 |
| ATOM | 53 | H6 | MAN | 10 | 8.106 | 24.963 | 28.688 | +0.03 | +0.49 | +0.424 | 3.976 |
| ATOM | 54 | C4 | MAN | 10 | 6.946 | 25.727 | 27.133 | −0.46 | +0.07 | +0.113 | 3.976 |
| ATOM | 55 | O4 | MAN | 10 | 6.354 | 25.987 | 28.395 | −0.46 | −0.23 | −0.537 | 3.976 |
| ATOM | 56 | HO4 | MAN | 10 | 5.330 | 25.906 | 28.317 | −0.27 | +0.05 | +0.424 | 3.976 |
| ATOM | 57 | C3 | MAN | 10 | 5.884 | 25.864 | 26.036 | −0.46 | +0.09 | +0.113 | 3.976 |
| ATOM | 58 | O3 | MAN | 10 | 4.958 | 24.792 | 26.134 | −0.13 | −0.47 | −0.537 | 3.976 |
| ATOM | 59 | HO3 | MAN | 10 | 3.997 | 25.165 | 26.122 | −0.03 | +0.46 | +0.424 | 3.976 |
| ATOM | 60 | C2 | MAN | 10 | 6.515 | 25.868 | 24.640 | −0.29 | +0.11 | +0.113 | 3.976 |
| ATOM | 61 | O2 | MAN | 10 | 7.040 | 24.584 | 24.340 | +0.01 | −0.43 | −0.537 | 3.976 |
| ATOM | 62 | HO2 | MAN | 10 | 6.508 | 23.867 | 24.855 | −0.19 | +0.22 | +0.424 | 3.976 |
| ATOM | 63 | C4 | MAN | 10 | 4.560 | 29.025 | 25.080 | −0.10 | +0.08 | +0.113 | 3.976 |
| ATOM | 64 | O4 | MAN | 10 | 5.310 | 29.968 | 25.832 | +0.20 | −0.31 | −0.537 | 3.976 |
| ATOM | 65 | HO4 | MAN | 10 | 6.073 | 29.485 | 26.328 | +0.11 | +0.21 | +0.424 | 3.976 |
| ATOM | 66 | C3 | MAN | 10 | 3.081 | 29.421 | 25.055 | −0.08 | +0.08 | +0.113 | 3.976 |
| ATOM | 67 | O3 | MAN | 10 | 2.529 | 29.626 | 26.385 | +0.19 | −0.15 | −0.227 | 3.976 |
| ATOM | 68 | C1 | BDG | 10 | 1.407 | 30.501 | 26.508 | −0.08 | +0.15 | +0.227 | 3.976 |
| ATOM | 69 | C2 | BDG | 10 | 1.357 | 31.093 | 27.935 | −0.13 | +0.06 | +0.113 | 3.976 |
| ATOM | 70 | O22 | BDG | 10 | 0.326 | 32.082 | 27.990 | +0.21 | −0.07 | −0.113 | 3.976 |
| ATOM | 71 | C3 | BDG | 10 | 1.044 | 29.997 | 28.981 | −0.27 | +0.06 | +0.113 | 3.976 |
| ATOM | 72 | O3 | BDG | 10 | 0.855 | 30.583 | 30.245 | +0.08 | −0.17 | −0.537 | 3.976 |
| ATOM | 73 | HO3 | BDG | 10 | 0.691 | 29.857 | 30.875 | −0.19 | −0.03 | +0.424 | 3.976 |
| ATOM | 74 | C4 | BDG | 10 | −0.205 | 29.158 | 28.603 | −0.34 | +0.09 | +0.113 | 3.976 |
| ATOM | 75 | O4 | BDG | 10 | −0.331 | 28.081 | 29.502 | −0.11 | −0.55 | −0.537 | 3.976 |
| ATOM | 76 | HO4 | BDG | 10 | −1.136 | 27.593 | 29.252 | +0.06 | +0.68 | +0.424 | 3.976 |
| ATOM | 77 | C5 | BDG | 10 | −0.107 | 28.649 | 27.134 | −0.27 | +0.10 | +0.113 | 3.976 |
| ATOM | 78 | C6 | BDG | 10 | −1.406 | 27.962 | 26.623 | −0.40 | +0.13 | +0.113 | 3.976 |
| ATOM | 79 | O6 | BDG | 10 | −1.660 | 26.760 | 27.318 | +0.18 | −0.83 | −0.537 | 3.976 |
| ATOM | 80 | H6 | BDG | 10 | −2.464 | 26.392 | 26.946 | −0.13 | +0.50 | +0.424 | 3.976 |
| ATOM | 81 | O | BDG | 10 | 0.187 | 29.752 | 26.240 | +0.19 | −0.18 | −0.227 | 3.976 |
| ATOM | 82 | C2 | MAN | 10 | 2.283 | 28.377 | 24.260 | −0.16 | +0.09 | +0.113 | 3.976 |
| ATOM | 83 | O2 | MAN | 10 | 2.368 | 27.117 | 24.910 | +0.05 | −0.56 | −0.537 | 3.976 |
| ATOM | 84 | HO2 | MAN | 10 | 1.649 | 26.485 | 24.531 | +0.07 | +0.45 | +0.424 | 3.976 |
| ATOM | 85 | C4 | MAN | 10 | 2.878 | 27.405 | 19.430 | −0.34 | +0.08 | +0.113 | 3.976 |
| ATOM | 86 | O4 | MAN | 10 | 1.956 | 26.334 | 19.577 | −0.03 | −0.27 | −0.537 | 3.976 |
| ATOM | 87 | HO4 | MAN | 10 | 1.030 | 26.710 | 19.824 | +0.01 | +0.18 | +0.424 | 3.976 |
| TER | | | | | | | | | | | |
| ENDMDL | | | | | | | | | | | |
| MODEL | 84 | | | | | | | | | | |
| USER | Run = 84 | | | | | | | | | | |
| USER | Cluster Rank = 11 | | | | | | | | | | |
| USER | Number of conformations in this cluster = 1 | | | | | | | | | | |
| USER | | | | | | | | | | | |
| USER | RMSD from reference structure = 3.445 A | | | | | | | | | | |
| USER | | | | | | | | | | | |
| USER | Estimated Free Energy of Binding = −0.46 kcal/mol [= (1) + (3)] | | | | | | | | | | |
| USER | Estimated Inhibition Constant, Ki = +0.46 [Temperature = 298.15 K] | | | | | | | | | | |
| USER | | | | | | | | | | | |
| USER | Final Docked Energy = −2.05 kcal/mol [= (1) + (2)] | | | | | | | | | | |
| USER | | | | | | | | | | | |
| USER | (1) Final Intermolecular Energy = −5.44 kcal/mol | | | | | | | | | | |
| USER | (2) Final Internal Energy of Ligand = +3.39 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 4.347370 27.163730 21.896105 | | | | | | | | | | |
| USER | NEWDPF quat0 0.996282 0.076147 0.040306 43.401175 | | | | | | | | | | |
| USER | NEWDPF ndihe 16 | | | | | | | | | | |
| USER | NEWDPF dihe0 −52.06 −10.45 179.33 −19.96 60.12 116.71 −4.34 −92.00 | | | | | | | | | | |
| | 108.31 −87.78 −5.82 63.85 109.95 26.07 −10.80 −8.04 | | | | | | | | | | |
| USER | | | | | | | | | | | |
| USER | | | | Rank | x | y | z | vdW | Elec | q | RMS |
| ATOM | 1 | C5 | MAN | 11 | 8.097 | 24.685 | 16.577 | −0.37 | +0.14 | +0.113 | 3.445 |
| ATOM | 2 | HO4 | MAN | 11 | 9.007 | 24.169 | 19.042 | +0.10 | +0.40 | +0.424 | 3.445 |
| ATOM | 3 | O4 | MAN | 11 | 9.111 | 23.416 | 18.346 | +0.05 | −0.56 | −0.537 | 3.445 |
| ATOM | 4 | O5 | MAN | 11 | 6.865 | 24.888 | 15.848 | −0.01 | −0.38 | −0.227 | 3.445 |
| ATOM | 5 | HO2 | MAN | 11 | 4.347 | 22.577 | 16.199 | +0.01 | +0.46 | +0.424 | 3.445 |
| ATOM | 6 | O2 | MAN | 11 | 5.330 | 22.620 | 16.501 | +0.03 | −0.58 | −0.537 | 3.445 |
| ATOM | 7 | C2 | MAN | 11 | 5.496 | 23.683 | 17.419 | −0.31 | +0.13 | +0.113 | 3.445 |
| ATOM | 8 | C1 | MAN | 11 | 5.698 | 24.987 | 16.668 | −0.09 | +0.35 | +0.227 | 3.445 |
| ATOM | 9 | C4 | MAN | 11 | 8.003 | 23.432 | 17.463 | −0.36 | +0.12 | +0.113 | 3.445 |
| ATOM | 10 | O3 | MAN | 11 | 6.569 | 22.166 | 18.939 | +0.06 | −0.53 | −0.537 | 3.445 |
| ATOM | 11 | HO3 | MAN | 11 | 5.764 | 22.199 | 19.581 | +0.10 | +0.45 | +0.424 | 3.445 |
| ATOM | 12 | C3 | MAN | 11 | 6.713 | 23.421 | 18.295 | −0.26 | +0.12 | +0.113 | 3.445 |
| ATOM | 13 | C6 | MAN | 11 | 9.212 | 24.580 | 15.551 | −0.53 | +0.15 | +0.113 | 3.445 |
| ATOM | 14 | O6 | MAN | 11 | 9.078 | 25.567 | 14.535 | −0.40 | −1.00 | −0.537 | 3.445 |
| ATOM | 15 | H6 | MAN | 11 | 8.584 | 26.386 | 14.915 | +0.07 | +0.65 | +0.424 | 3.445 |
| ATOM | 16 | O3 | MAN | 11 | 5.805 | 26.013 | 17.602 | +0.11 | −0.25 | −0.227 | 3.445 |
| ATOM | 17 | C3 | MAN | 11 | 4.678 | 26.891 | 17.679 | −0.34 | +0.11 | +0.113 | 3.445 |
| ATOM | 18 | C2 | MAN | 11 | 5.203 | 28.292 | 17.978 | −0.26 | +0.10 | +0.113 | 3.445 |
| ATOM | 19 | O2 | MAN | 11 | 6.117 | 28.247 | 19.066 | +0.19 | −0.47 | −0.537 | 3.445 |
| ATOM | 20 | HO2 | MAN | 11 | 6.076 | 29.135 | 19.586 | +0.11 | +0.35 | +0.424 | 3.445 |
| ATOM | 21 | C1 | MAN | 11 | 4.056 | 29.215 | 18.317 | −0.31 | +0.19 | +0.227 | 3.445 |
| ATOM | 22 | O4 | NAG | 11 | 4.554 | 30.455 | 18.667 | +0.11 | −0.19 | −0.227 | 3.445 |
| ATOM | 23 | C4 | NAG | 11 | 3.759 | 31.604 | 18.346 | −0.31 | +0.10 | +0.113 | 3.445 |
| ATOM | 24 | C5 | NAG | 11 | 4.573 | 32.873 | 18.675 | −0.20 | +0.09 | +0.113 | 3.445 |
| ATOM | 25 | C6 | NAG | 11 | 5.656 | 33.195 | 17.658 | −0.14 | +0.11 | +0.113 | 3.445 |
| ATOM | 26 | O6 | NAG | 11 | 5.385 | 34.404 | 16.961 | +0.08 | −0.52 | −0.537 | 3.445 |
| ATOM | 27 | H6 | NAG | 11 | 4.829 | 35.029 | 17.561 | +0.10 | +0.32 | +0.424 | 3.445 |
| ATOM | 28 | O5 | NAG | 11 | 3.722 | 34.011 | 18.704 | +0.13 | −0.17 | −0.227 | 3.445 |
| ATOM | 29 | C1 | NAG | 11 | 2.726 | 33.907 | 19.702 | −0.17 | +0.08 | +0.113 | 3.445 |
| ATOM | 30 | C2 | NAG | 11 | 1.726 | 32.836 | 19.236 | −0.27 | +0.22 | +0.211 | 3.445 |
| ATOM | 31 | N2 | NAG | 11 | 0.621 | 32.705 | 20.159 | −0.12 | −0.64 | −0.650 | 3.445 |
| ATOM | 32 | HN2 | NAG | 11 | 0.748 | 32.950 | 21.150 | +0.11 | +0.35 | +0.440 | 3.445 |
| ATOM | 33 | C7 | NAG | 11 | −0.562 | 32.269 | 19.732 | −0.28 | +0.49 | +0.396 | 3.445 |
| ATOM | 34 | O7 | NAG | 11 | −0.779 | 31.960 | 18.561 | +0.02 | −0.84 | −0.396 | 3.445 |
| ATOM | 35 | C8 | NAG | 11 | −1.678 | 32.165 | 20.756 | −0.18 | +0.00 | +0.000 | 3.445 |
| ATOM | 36 | C3 | NAG | 11 | 2.460 | 31.503 | 19.148 | −0.27 | +0.10 | +0.113 | 3.445 |
| ATOM | 37 | O3 | NAG | 11 | 1.621 | 30.530 | 18.546 | −0.01 | −0.49 | −0.537 | 3.445 |
| ATOM | 38 | HO3 | NAG | 11 | 1.518 | 30.736 | 17.542 | +0.06 | +0.44 | +0.424 | 3.445 |
| ATOM | 39 | O5 | MAN | 11 | 3.305 | 28.674 | 19.415 | +0.10 | −0.18 | −0.227 | 3.445 |
| ATOM | 40 | C5 | MAN | 11 | 2.639 | 27.463 | 19.041 | −0.34 | +0.08 | +0.113 | 3.445 |
| ATOM | 41 | C6 | MAN | 11 | 1.558 | 27.166 | 20.089 | −0.39 | +0.06 | +0.113 | 3.445 |
| ATOM | 42 | O6 | MAN | 11 | 1.766 | 27.924 | 21.310 | +0.09 | −0.15 | −0.227 | 3.445 |
| ATOM | 43 | C1 | MAN | 11 | 2.156 | 27.160 | 22.433 | −0.08 | +0.18 | +0.227 | 3.445 |
| ATOM | 44 | O5 | MAN | 11 | 3.548 | 26.798 | 22.463 | +0.17 | −0.33 | −0.227 | 3.445 |
| ATOM | 45 | C5 | MAN | 11 | 4.468 | 27.873 | 22.785 | −0.14 | +0.11 | +0.113 | 3.445 |
| ATOM | 46 | C6 | MAN | 11 | 5.894 | 27.340 | 22.683 | −0.11 | +0.11 | +0.113 | 3.445 |
| ATOM | 47 | O6 | MAN | 11 | 5.887 | 25.922 | 22.421 | +0.13 | −0.33 | −0.227 | 3.445 |
| ATOM | 48 | C1 | MAN | 11 | 6.400 | 25.165 | 23.486 | −0.19 | +0.31 | +0.227 | 3.445 |
| ATOM | 49 | O5 | MAN | 11 | 6.345 | 23.757 | 23.239 | +0.01 | −0.31 | −0.227 | 3.445 |
| ATOM | 50 | C5 | MAN | 11 | 7.082 | 23.325 | 22.070 | −0.38 | +0.12 | +0.113 | 3.445 |
| ATOM | 51 | C6 | MAN | 11 | 6.922 | 21.822 | 21.969 | −0.47 | +0.13 | +0.113 | 3.445 |
| ATOM | 52 | O6 | MAN | 11 | 5.567 | 21.461 | 21.740 | −0.12 | −0.89 | −0.537 | 3.445 |
| ATOM | 53 | H6 | MAN | 11 | 4.949 | 22.091 | 22.272 | +0.06 | +0.81 | +0.424 | 3.445 |
| ATOM | 54 | C4 | MAN | 11 | 8.558 | 23.723 | 22.176 | −0.29 | +0.09 | +0.113 | 3.445 |
| ATOM | 55 | O4 | MAN | 11 | 9.179 | 23.483 | 20.924 | +0.07 | −0.45 | −0.537 | 3.445 |
| ATOM | 56 | HO4 | MAN | 11 | 9.838 | 24.246 | 20.715 | +0.11 | +0.34 | +0.424 | 3.445 |
| ATOM | 57 | C3 | MAN | 11 | 8.731 | 25.199 | 22.552 | −0.13 | +0.09 | +0.113 | 3.445 |
| ATOM | 58 | O3 | MAN | 11 | 10.092 | 25.455 | 22.865 | +0.20 | −0.36 | −0.537 | 3.445 |
| ATOM | 59 | HO3 | MAN | 11 | 10.415 | 26.285 | 22.345 | +0.12 | +0.29 | +0.424 | 3.445 |
| ATOM | 60 | C2 | MAN | 11 | 7.852 | 25.581 | 23.748 | −0.20 | +0.09 | +0.113 | 3.445 |
| ATOM | 61 | O2 | MAN | 11 | 8.333 | 24.950 | 24.925 | +0.08 | −0.32 | −0.537 | 3.445 |
| ATOM | 62 | HO2 | MAN | 11 | 9.347 | 24.788 | 24.838 | +0.10 | +0.25 | +0.424 | 3.445 |
| ATOM | 63 | C4 | MAN | 11 | 4.130 | 28.465 | 24.157 | −0.12 | +0.10 | +0.113 | 3.445 |
| ATOM | 64 | O4 | MAN | 11 | 5.038 | 29.512 | 24.472 | +0.22 | −0.37 | −0.537 | 3.445 |
| ATOM | 65 | HO4 | MAN | 11 | 5.790 | 29.147 | 25.074 | +0.12 | +0.27 | +0.424 | 3.445 |
| ATOM | 66 | C3 | MAN | 11 | 2.704 | 29.011 | 24.060 | −0.10 | +0.09 | +0.113 | 3.445 |
| ATOM | 67 | O3 | MAN | 11 | 2.299 | 29.749 | 25.246 | +0.21 | −0.16 | −0.227 | 3.445 |
| ATOM | 68 | C1 | BDG | 11 | 1.553 | 30.952 | 25.053 | −0.03 | +0.16 | +0.227 | 3.445 |
| ATOM | 69 | C2 | BDG | 11 | 2.520 | 32.152 | 24.918 | −0.01 | +0.07 | +0.113 | 3.445 |
| ATOM | 70 | O22 | BDG | 11 | 1.766 | 33.315 | 24.570 | +0.23 | −0.07 | −0.113 | 3.445 |
| ATOM | 71 | C3 | BDG | 11 | 3.259 | 32.423 | 26.250 | −0.03 | +0.06 | +0.113 | 3.445 |
| ATOM | 72 | O3 | BDG | 11 | 4.012 | 33.605 | 26.145 | +0.23 | −0.29 | −0.537 | 3.445 |
| ATOM | 73 | HO3 | BDG | 11 | 4.482 | 33.721 | 26.992 | +0.12 | +0.21 | +0.424 | 3.445 |
| ATOM | 74 | C4 | BDG | 11 | 2.287 | 32.530 | 27.454 | −0.06 | +0.06 | +0.113 | 3.445 |
| ATOM | 75 | O4 | BDG | 11 | 3.031 | 32.608 | 28.647 | +0.17 | −0.25 | −0.537 | 3.445 |
| ATOM | 76 | HO4 | BDG | 11 | 2.393 | 32.700 | 29.377 | +0.10 | +0.19 | +0.424 | 3.445 |
| ATOM | 77 | C5 | BDG | 11 | 1.308 | 31.318 | 27.487 | −0.10 | +0.07 | +0.113 | 3.445 |
| ATOM | 78 | C6 | BDG | 11 | 0.183 | 31.452 | 28.553 | −0.13 | +0.06 | +0.113 | 3.445 |
| ATOM | 79 | O6 | BDG | 11 | 0.709 | 31.420 | 29.862 | +0.11 | −0.23 | −0.537 | 3.445 |
| ATOM | 80 | H6 | BDG | 11 | −0.037 | 31.498 | 30.460 | +0.10 | +0.18 | +0.424 | 3.445 |
| ATOM | 81 | O | BDG | 11 | 0.671 | 31.154 | 26.194 | +0.22 | −0.15 | −0.227 | 3.445 |
| ATOM | 82 | C2 | MAN | 11 | 1.735 | 27.861 | 23.744 | −0.25 | +0.10 | +0.113 | 3.445 |
| ATOM | 83 | O2 | MAN | 11 | 1.751 | 26.920 | 24.807 | +0.02 | −0.54 | −0.537 | 3.445 |
| ATOM | 84 | HO2 | MAN | 11 | 0.940 | 26.290 | 24.722 | +0.07 | +0.40 | +0.424 | 3.445 |
| ATOM | 85 | C4 | MAN | 11 | 3.692 | 26.382 | 18.759 | −0.32 | +0.10 | +0.113 | 3.445 |
| ATOM | 86 | O4 | MAN | 11 | 3.050 | 25.202 | 18.297 | +0.00 | −0.44 | −0.537 | 3.445 |
| ATOM | 87 | HO4 | MAN | 11 | 2.030 | 25.343 | 18.298 | +0.07 | +0.31 | +0.424 | 3.445 |
| TER | | | | | | | | | | | |
| ENDMDL | | | | | | | | | | | |
| MODEL | 55 | | | | | | | | | | |
| USER | Run = 55 | | | | | | | | | | |
| USER | Cluster Rank = 12 | | | | | | | | | | |
| USER | Number of conformations in this cluster = 1 | | | | | | | | | | |
| USER | | | | | | | | | | | |
| USER | RMSD from reference structure = 3.732 A | | | | | | | | | | |
| USER | | | | | | | | | | | |
| USER | Estimated Free Energy of Binding = +0.22 kcal/mol [= (1) + (3)] | | | | | | | | | | |
| USER | Estimated Inhibition Constant, Ki = +1.45 [Temperature = 298.15 K] | | | | | | | | | | |
| USER | | | | | | | | | | | |
| USER | Final Docked Energy = −2.03 kcal/mol [= (1) + (2)] | | | | | | | | | | |
| USER | | | | | | | | | | | |
| USER | (1) Final Intermolecular Energy = −4.76 kcal/mol | | | | | | | | | | |
| USER | (2) Final Internal Energy of Ligand = +2.72 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 −1.041717 29.565840 16.927652 | | | | | | | | | | |
| USER | NEWDPF quat0 0.695145 0.716580 −0.057317 −179.977341 | | | | | | | | | | |
| USER | NEWDPF ndihe 16 | | | | | | | | | | |
| USER | NSWDPF dihe0 122.95 52.93 180.00 −34.66 48.68 36.86 −17.57 −65.43 −74.44 | | | | | | | | | | |
| | −125.42 118.88 123.27 35.01 20.90 21.35 −28.16 | | | | | | | | | | |
| USER | | | | | | | | | | | |
| USER | | | | Rank | x | y | z | vdW | Elec | q | RMS |
| ATOM | 1 | C5 | MAN | 12 | −6.471 | 33.404 | 18.996 | −0.13 | +0.10 | +0.113 | 3.732 |
| ATOM | 2 | HO4 | MAN | 12 | −5.371 | 34.037 | 16.639 | +0.09 | +0.43 | +0.424 | 3.732 |
| ATOM | 3 | O4 | MAN | 12 | −6.398 | 34.104 | 16.700 | +0.08 | −0.49 | −0.537 | 3.732 |
| ATOM | 4 | O5 | MAN | 12 | −6.714 | 32.271 | 19.861 | +0.17 | −0.24 | −0.227 | 3.732 |
| ATOM | 5 | HO2 | MAN | 12 | −8.178 | 29.466 | 18.523 | +0.06 | +0.56 | +0.424 | 3.732 |
| ATOM | 6 | O2 | MAN | 12 | −8.001 | 30.419 | 18.177 | −0.07 | −0.70 | −0.537 | 3.732 |
| ATOM | 7 | C2 | MAN | 12 | −6.606 | 30.639 | 18.094 | −0.46 | +0.13 | +0.113 | 3.732 |
| ATOM | 8 | C1 | MAN | 12 | −6.071 | 31.062 | 19.451 | −0.29 | +0.27 | +0.227 | 3.732 |
| ATOM | 9 | C4 | MAN | 12 | −6.888 | 33.083 | 17.551 | −0.24 | +0.11 | +0.113 | 3.732 |
| ATOM | 10 | O3 | MAN | 12 | −6.884 | 31.387 | 15.831 | +0.06 | −0.72 | −0.537 | 3.732 |
| ATOM | 11 | HO3 | MAN | 12 | −6.420 | 30.542 | 15.467 | +0.05 | +0.62 | +0.424 | 3.732 |
| ATOM | 12 | C3 | MAN | 12 | −6.317 | 31.737 | 17.082 | −0.45 | +0.13 | +0.113 | 3.732 |
| ATOM | 13 | C6 | MAN | 12 | −7.247 | 34.578 | 19.570 | −0.06 | +0.09 | +0.113 | 3.732 |
| ATOM | 14 | O6 | MAN | 12 | −6.379 | 35.524 | 20.182 | +0.22 | −0.36 | −0.537 | 3.732 |
| ATOM | 15 | H6 | MAN | 12 | −6.909 | 36.084 | 20.865 | +0.12 | +0.27 | +0.424 | 3.732 |
| ATOM | 16 | O3 | MAN | 12 | −4.692 | 31.217 | 19.343 | +0.09 | −0.26 | −0.227 | 3.732 |
| ATOM | 17 | C3 | MAN | 12 | −3.917 | 30.511 | 20.316 | −0.17 | +0.15 | +0.113 | 3.732 |
| ATOM | 18 | C2 | MAN | 12 | −3.249 | 31.545 | 21.215 | −0.13 | +0.15 | +0.113 | 3.732 |
| ATOM | 19 | O2 | MAN | 12 | −2.617 | 32.543 | 20.424 | +0.15 | −0.55 | −0.537 | 3.732 |
| ATOM | 20 | HO2 | MAN | 12 | −1.794 | 32.908 | 20.925 | +0.11 | +0.39 | +0.424 | 3.732 |
| ATOM | 21 | C1 | MAN | 12 | −2.224 | 30.878 | 22.104 | −0.04 | +0.29 | +0.227 | 3.732 |
| ATOM | 22 | O4 | NAG | 12 | −1.571 | 31.838 | 22.851 | +0.18 | −0.22 | −0.227 | 3.732 |
| ATOM | 23 | C4 | NAG | 12 | −1.834 | 31.876 | 24.259 | −0.09 | +0.10 | +0.113 | 3.732 |
| ATOM | 24 | C5 | NAG | 12 | −1.753 | 33.342 | 24.736 | −0.03 | +0.08 | +0.113 | 3.732 |
| ATOM | 25 | C6 | NAG | 12 | −2.966 | 34.181 | 24.368 | −0.03 | +0.08 | +0.113 | 3.732 |
| ATOM | 26 | O6 | NAG | 12 | −3.696 | 34.587 | 25.519 | +0.23 | −0.36 | −0.537 | 3.732 |
| ATOM | 27 | H6 | NAG | 12 | −3.057 | 34.657 | 26.324 | +0.12 | +0.26 | +0.424 | 3.732 |
| ATOM | 28 | O5 | NAG | 12 | −1.662 | 33.395 | 26.153 | +0.23 | −0.15 | −0.227 | 3.732 |
| ATOM | 29 | C1 | NAG | 12 | −0.500 | 32.753 | 26.638 | −0.02 | +0.07 | +0.113 | 3.732 |
| ATOM | 30 | C2 | NAG | 12 | −0.698 | 31.242 | 26.438 | −0.06 | +0.16 | +0.211 | 3.732 |
| ATOM | 31 | N2 | NAG | 12 | 0.411 | 30.485 | 26.974 | −0.04 | −0.45 | −0.650 | 3.732 |
| ATOM | 32 | HN2 | NAG | 12 | 1.375 | 30.820 | 26.835 | +0.11 | +0.28 | +0.440 | 3.732 |
| ATOM | 33 | C7 | NAG | 12 | 0.194 | 29.358 | 27.646 | −0.22 | +0.31 | +0.396 | 3.732 |
| ATOM | 34 | O7 | NAG | 12 | −0.932 | 28.905 | 27.846 | +0.08 | −0.39 | −0.396 | 3.732 |
| ATOM | 35 | C8 | NAG | 12 | 1.407 | 28.622 | 28.185 | −0.37 | +0.00 | +0.000 | 3.732 |
| ATOM | 36 | C3 | NAG | 12 | −0.811 | 30.968 | 24.944 | −0.08 | +0.10 | +0.113 | 3.732 |
| ATOM | 37 | O3 | NAG | 12 | −1.179 | 29.614 | 24.728 | +0.13 | −0.56 | −0.537 | 3.732 |
| ATOM | 38 | HO3 | NAG | 12 | −2.162 | 29.476 | 25.003 | +0.09 | +0.54 | +0.424 | 3.732 |
| ATOM | 39 | O5 | MAN | 12 | −1.272 | 30.168 | 21.297 | +0.11 | −0.23 | −0.227 | 3.732 |
| ATOM | 40 | C5 | MAN | 12 | −1.883 | 29.060 | 20.627 | −0.43 | +0.12 | +0.113 | 3.732 |
| ATOM | 41 | C6 | MAN | 12 | −0.771 | 28.127 | 20.130 | −0.26 | +0.08 | +0.113 | 3.732 |
| ATOM | 42 | O6 | MAN | 12 | 0.499 | 28.818 | 20.004 | +0.00 | −0.17 | −0.227 | 3.732 |
| ATOM | 43 | C1 | MAN | 12 | 1.600 | 27.998 | 19.668 | −0.38 | +0.15 | +0.227 | 3.732 |
| ATOM | 44 | O5 | MAN | 12 | 2.502 | 28.567 | 18.702 | +0.02 | −0.16 | −0.227 | 3.732 |
| ATOM | 45 | C5 | MAN | 12 | 3.354 | 29.633 | 19.192 | −0.25 | +0.09 | +0.113 | 3.732 |
| ATOM | 46 | C6 | MAN | 12 | 4.172 | 30.178 | 18.025 | −0.33 | +0.10 | +0.113 | 3.732 |
| ATOM | 47 | O6 | MAN | 12 | 3.890 | 31.576 | 17.817 | +0.01 | −0.20 | −0.227 | 3.732 |
| ATOM | 48 | C1 | MAN | 12 | 4.271 | 32.380 | 18.903 | −0.19 | +0.18 | +0.227 | 3.732 |
| ATOM | 49 | O5 | MAN | 12 | 5.542 | 33.005 | 18.707 | +0.15 | −0.19 | −0.227 | 3.732 |
| ATOM | 50 | C5 | MAN | 12 | 5.614 | 33.868 | 17.547 | −0.21 | +0.10 | +0.113 | 3.732 |
| ATOM | 51 | C6 | MAN | 12 | 7.014 | 34.445 | 17.501 | −0.17 | +0.11 | +0.113 | 3.732 |
| ATOM | 52 | O6 | MAN | 12 | 7.984 | 33.430 | 17.285 | +0.14 | −0.60 | −0.537 | 3.732 |
| ATOM | 53 | H6 | MAN | 12 | 7.708 | 32.577 | 17.792 | +0.10 | +0.44 | +0.424 | 3.732 |
| ATOM | 54 | C4 | MAN | 12 | 4.556 | 34.975 | 17.622 | −0.30 | +0.08 | +0.113 | 3.732 |
| ATOM | 55 | O4 | MAN | 12 | 4.517 | 35.645 | 16.373 | +0.04 | −0.35 | −0.537 | 3.732 |
| ATOM | 56 | HO4 | MAN | 12 | 3.540 | 35.862 | 16.130 | −0.25 | +0.01 | +0.424 | 3.732 |
| ATOM | 57 | C3 | MAN | 12 | 3.163 | 34.419 | 17.943 | −0.40 | +0.08 | +0.113 | 3.732 |
| ATOM | 58 | O3 | MAN | 12 | 2.278 | 35.491 | 18.232 | −0.03 | −0.24 | −0.537 | 3.732 |
| ATOM | 59 | HO3 | MAN | 12 | 1.415 | 35.383 | 17.679 | −0.09 | +0.11 | +0.424 | 3.732 |
| ATOM | 60 | C2 | MAN | 12 | 3.203 | 33.455 | 19.134 | −0.21 | +0.09 | +0.113 | 3.732 |
| ATOM | 61 | O2 | MAN | 12 | 3.484 | 34.170 | 20.328 | +0.20 | −0.35 | −0.537 | 3.732 |
| ATOM | 62 | HO2 | MAN | 12 | 3.146 | 35.139 | 20.236 | +0.11 | +0.25 | +0.424 | 3.732 |
| ATOM | 63 | C4 | MAN | 12 | 4.209 | 29.133 | 20.361 | −0.11 | +0.09 | +0.113 | 3.732 |
| ATOM | 64 | O4 | MAN | 12 | 5.047 | 30.177 | 20.833 | +0.22 | −0.41 | −0.537 | 3.732 |
| ATOM | 65 | HO4 | MAN | 12 | 5.983 | 30.085 | 20.413 | +0.12 | +0.33 | +0.424 | 3.732 |
| ATOM | 66 | C3 | MAN | 12 | 3.236 | 28.695 | 21.459 | −0.13 | +0.09 | +0.113 | 3.732 |
| ATOM | 67 | O3 | MAN | 12 | 3.907 | 28.341 | 22.699 | +0.18 | −0.20 | −0.227 | 3.732 |
| ATOM | 68 | C1 | BDG | 12 | 3.482 | 29.002 | 23.893 | −0.09 | +0.18 | +0.227 | 3.732 |
| ATOM | 69 | C2 | BDG | 12 | 4.475 | 30.133 | 24.248 | −0.03 | +0.08 | +0.113 | 3.732 |
| ATOM | 70 | O22 | BDG | 12 | 3.951 | 30.880 | 25.348 | +0.22 | −0.07 | −0.113 | 3.732 |
| ATOM | 71 | C3 | BDG | 12 | 5.852 | 29.557 | 24.655 | −0.05 | +0.07 | +0.113 | 3.732 |
| ATOM | 72 | O3 | BDG | 12 | 6.684 | 30.591 | 25.116 | +0.22 | −0.31 | −0.537 | 3.732 |
| ATOM | 73 | HO3 | BDG | 12 | 7.546 | 30.190 | 25.332 | +0.12 | +0.23 | +0.424 | 3.732 |
| ATOM | 74 | C4 | BDG | 12 | 5.735 | 28.457 | 25.742 | −0.14 | +0.07 | +0.113 | 3.732 |
| ATOM | 75 | O4 | BDG | 12 | 6.996 | 27.864 | 25.941 | +0.17 | −0.30 | −0.537 | 3.732 |
| ATOM | 76 | HO4 | BDG | 12 | 6.892 | 27.199 | 26.646 | +0.10 | +0.20 | +0.424 | 3.732 |
| ATOM | 77 | C5 | BDG | 12 | 4.674 | 27.389 | 25.340 | −0.25 | +0.10 | +0.113 | 3.732 |
| ATOM | 78 | C6 | BDG | 12 | 4.365 | 26.360 | 26.465 | −0.37 | +0.08 | +0.113 | 3.732 |
| ATOM | 79 | O6 | BDG | 12 | 5.486 | 25.549 | 26.742 | −0.08 | −0.31 | −0.537 | 3.732 |
| ATOM | 80 | H6 | BDG | 12 | 5.220 | 24.939 | 27.433 | −0.18 | +0.09 | +0.424 | 3.732 |
| ATOM | 81 | O | BDG | 12 | 3.426 | 28.034 | 24.979 | +0.15 | −0.20 | −0.227 | 3.732 |
| ATOM | 82 | C2 | MAN | 12 | 2.356 | 27.549 | 20.938 | −0.27 | +0.08 | +0.113 | 3.732 |
| ATOM | 83 | O2 | MAN | 12 | 3.170 | 26.427 | 20.629 | +0.08 | −0.43 | −0.537 | 3.732 |
| ATOM | 84 | HO2 | MAN | 12 | 2.577 | 25.591 | 20.519 | −0.18 | +0.11 | +0.424 | 3.732 |
| ATOM | 85 | C4 | MAN | 12 | −2.894 | 29.593 | 19.603 | −0.42 | +0.12 | +0.113 | 3.732 |
| ATOM | 86 | O4 | MAN | 12 | −3.576 | 28.504 | 18.996 | −0.12 | −0.45 | −0.537 | 3.732 |
| ATOM | 87 | HO4 | MAN | 12 | −3.201 | 27.618 | 19.362 | −0.01 | +0.32 | +0.424 | 3.732 |
| TER | | | | | | | | | | | |
| ENDMDL | | | | | | | | | | | |
| MODEL | 99 | | | | | | | | | | |
| USER | Run = 99 | | | | | | | | | | |
| USER | Cluster Rank = 13 | | | | | | | | | | |
| USER | Number of conformations in this cluster = 1 | | | | | | | | | | |
| USER | | | | | | | | | | | |
| USER | RMSD from reference structure = 4.594 A | | | | | | | | | | |
| USER | | | | | | | | | | | |
| USER | Estimated Free Energy of Binding = −0.77 kcal/mol [= (1) + (3)] | | | | | | | | | | |
| USER | Estimated Inhibition Constant, Ki = +0.27 [Temperature = 298.15 K] | | | | | | | | | | |
| USER | | | | | | | | | | | |
| USER | Final Docked Energy = −1.88 kcal/mol [= (1) + (2)] | | | | | | | | | | |
| USER | | | | | | | | | | | |
| USER | (1) Final Intermolecular Energy = −5.75 kcal/mol | | | | | | | | | | |
| USER | (2) Final Internal Energy of Ligand = +3.87 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 −1.365723 29.931822 18.699576 | | | | | | | | | | |
| USER | NEWDPF quat0 −0.883084 0.137804 0.448522 146.061609 | | | | | | | | | | |
| USER | NEWDPF ndihe 16 | | | | | | | | | | |
| USER | NEWDPF dihe0 115.37 −13.69 76.94 −82.80 4.79 −1.12 −12.15 −11.97 −79.35 | | | | | | | | | | |
| | −59.31 84.08 148.76 24.56 158.71 −14.48 −68.21 | | | | | | | | | | |
| USER | | | | | | | | | | | |
| USER | | | | Rank | x | y | z | vdW | Elec | q | RMS |
| ATOM | 1 | C5 | MAN | 13 | 4.773 | 33.218 | 18.593 | −0.19 | +0.09 | +0.113 | 4.594 |
| ATOM | 2 | HO4 | MAN | 13 | 3.194 | 33.536 | 16.454 | +0.04 | +0.43 | +0.424 | 4.594 |
| ATOM | 3 | O4 | MAN | 13 | 3.727 | 34.364 | 16.760 | −0.02 | −0.44 | −0.537 | 4.594 |
| ATOM | 4 | O5 | MAN | 13 | 4.709 | 33.006 | 20.023 | +0.21 | −0.16 | −0.227 | 4.594 |
| ATOM | 5 | HO2 | MAN | 13 | 2.721 | 35.081 | 21.906 | +0.12 | +0.24 | +0.424 | 4.594 |
| ATOM | 6 | O2 | MAN | 13 | 3.028 | 35.077 | 20.923 | +0.21 | −0.31 | −0.537 | 4.594 |
| ATOM | 7 | C2 | MAN | 13 | 2.503 | 33.938 | 20.269 | −0.13 | +0.08 | +0.113 | 4.594 |
| ATOM | 8 | C1 | MAN | 13 | 3.398 | 32.738 | 20.525 | −0.08 | +0.17 | +0.227 | 4.594 |
| ATOM | 9 | C4 | MAN | 13 | 3.834 | 34.362 | 18.172 | −0.30 | +0.08 | +0.113 | 4.594 |
| ATOM | 10 | O3 | MAN | 13 | 1.662 | 35.359 | 18.527 | +0.00 | −0.20 | −0.537 | 4.594 |
| ATOM | 11 | HO3 | MAN | 13 | 0.691 | 35.201 | 18.831 | −0.21 | +0.05 | +0.424 | 4.594 |
| ATOM | 12 | C3 | MAN | 13 | 2.430 | 34.193 | 18.771 | −0.33 | +0.09 | +0.113 | 4.594 |
| ATOM | 13 | C6 | MAN | 13 | 6.223 | 33.511 | 18.250 | −0.16 | +0.10 | +0.113 | 4.594 |
| ATOM | 14 | O6 | MAN | 13 | 7.110 | 32.679 | 18.988 | +0.20 | −0.44 | −0.537 | 4.594 |
| ATOM | 15 | H6 | MAN | 13 | 8.065 | 33.057 | 18.928 | +0.12 | +0.35 | +0.424 | 4.594 |
| ATOM | 16 | O3 | MAN | 13 | 2.822 | 31.629 | 19.912 | +0.17 | −0.18 | −0.227 | 4.594 |
| ATOM | 17 | C3 | MAN | 13 | 3.386 | 30.366 | 20.274 | −0.12 | +0.09 | +0.113 | 4.594 |
| ATOM | 18 | C2 | MAN | 13 | 3.262 | 29.441 | 19.068 | −0.27 | +0.09 | +0.113 | 4.594 |
| ATOM | 19 | O2 | MAN | 13 | 1.936 | 29.483 | 18.557 | +0.01 | −0.40 | −0.537 | 4.594 |
| ATOM | 20 | HO2 | MAN | 13 | 1.701 | 28.571 | 18.138 | +0.07 | +0.24 | +0.424 | 4.594 |
| ATOM | 21 | C1 | MAN | 13 | 3.610 | 28.024 | 19.462 | −0.24 | +0.18 | +0.227 | 4.594 |
| ATOM | 22 | O4 | NAG | 13 | 3.413 | 27.187 | 18.381 | +0.04 | −0.19 | −0.227 | 4.594 |
| ATOM | 23 | C4 | NAG | 13 | 3.770 | 25.808 | 18.536 | −0.34 | +0.10 | +0.113 | 4.594 |
| ATOM | 24 | C5 | NAG | 13 | 5.215 | 25.613 | 18.029 | −0.25 | +0.12 | +0.113 | 4.594 |
| ATOM | 25 | C6 | NAG | 13 | 6.281 | 26.118 | 18.987 | −0.12 | +0.11 | +0.113 | 4.594 |
| ATOM | 26 | O6 | NAG | 13 | 7.071 | 25.055 | 19.506 | +0.19 | −0.52 | −0.537 | 4.594 |
| ATOM | 27 | H6 | NAG | 13 | 7.101 | 24.284 | 18.823 | +0.11 | +0.42 | +0.424 | 4.594 |
| ATOM | 28 | O5 | NAG | 13 | 5.489 | 24.232 | 17.839 | +0.10 | −0.25 | −0.227 | 4.594 |
| ATOM | 29 | C1 | NAG | 13 | 4.644 | 23.648 | 16.868 | −0.34 | +0.14 | +0.113 | 4.594 |
| ATOM | 30 | C2 | NAG | 13 | 3.238 | 23.561 | 17.483 | −0.42 | +0.18 | +0.211 | 4.594 |
| ATOM | 31 | N2 | NAG | 13 | 2.308 | 22.905 | 16.591 | −0.32 | −0.50 | −0.650 | 4.594 |
| ATOM | 32 | HN2 | NAG | 13 | 1.777 | 23.461 | 15.906 | +0.07 | +0.43 | +0.440 | 4.594 |
| ATOM | 33 | C7 | NAG | 13 | 2.135 | 21.587 | 16.650 | −0.38 | +0.17 | +0.396 | 4.594 |
| ATOM | 34 | O7 | NAG | 13 | 2.738 | 20.868 | 17.445 | +0.08 | −0.14 | −0.396 | 4.594 |
| ATOM | 35 | C8 | NAG | 13 | 1.148 | 20.969 | 15.677 | −0.47 | +0.00 | +0.000 | 4.594 |
| ATOM | 36 | C3 | NAG | 13 | 2.747 | 24.977 | 17.759 | −0.45 | +0.10 | +0.113 | 4.594 |
| ATOM | 37 | O3 | NAG | 13 | 1.534 | 24.932 | 18.495 | −0.11 | −0.33 | −0.537 | 4.594 |
| ATOM | 38 | HO3 | NAG | 13 | 1.728 | 24.626 | 19.460 | +0.02 | +0.09 | +0.424 | 4.594 |
| ATOM | 39 | O5 | MAN | 13 | 2.776 | 27.604 | 20.552 | +0.09 | −0.17 | −0.227 | 4.594 |
| ATOM | 40 | C5 | MAN | 13 | 3.065 | 28.346 | 21.742 | −0.16 | +0.09 | +0.113 | 4.594 |
| ATOM | 41 | C6 | MAN | 13 | 2.458 | 27.595 | 22.935 | −0.25 | +0.10 | +0.113 | 4.594 |
| ATOM | 42 | O6 | MAN | 13 | 3.477 | 27.127 | 23.854 | +0.00 | −0.31 | −0.227 | 4.594 |
| ATOM | 43 | C1 | MAN | 13 | 3.007 | 26.745 | 25.131 | −0.33 | +0.27 | +0.227 | 4.594 |
| ATOM | 44 | O5 | MAN | 13 | 3.932 | 25.960 | 25.904 | −0.15 | −0.25 | −0.227 | 4.594 |
| ATOM | 45 | C5 | MAN | 13 | 5.060 | 26.685 | 26.458 | −0.40 | +0.07 | +0.113 | 4.594 |
| ATOM | 46 | C6 | MAN | 13 | 5.979 | 25.691 | 27.163 | −0.44 | +0.06 | +0.113 | 4.594 |
| ATOM | 47 | O6 | MAN | 13 | 6.886 | 25.085 | 26.221 | −0.03 | −0.14 | −0.227 | 4.594 |
| ATOM | 48 | C1 | MAN | 13 | 7.694 | 26.022 | 25.556 | −0.28 | +0.15 | +0.227 | 4.594 |
| ATOM | 49 | O5 | MAN | 13 | 8.510 | 26.781 | 26.453 | +0.12 | −0.13 | −0.227 | 4.594 |
| ATOM | 50 | C5 | MAN | 13 | 9.420 | 25.988 | 27.252 | −0.37 | +0.08 | +0.113 | 4.594 |
| ATOM | 51 | C6 | MAN | 13 | 10.202 | 26.947 | 28.126 | −0.31 | +0.08 | +0.113 | 4.594 |
| ATOM | 52 | O6 | MAN | 13 | 10.521 | 28.141 | 27.427 | +0.17 | −0.30 | −0.537 | 4.594 |
| ATOM | 53 | H6 | MAN | 13 | 10.740 | 27.917 | 26.446 | +0.12 | +0.25 | +0.424 | 4.594 |
| ATOM | 54 | C4 | MAN | 13 | 10.352 | 25.159 | 26.360 | −0.31 | +0.08 | +0.113 | 4.594 |
| ATOM | 55 | O4 | MAN | 13 | 11.059 | 24.243 | 27.178 | −0.09 | −0.49 | −0.537 | 4.594 |
| ATOM | 56 | HO4 | MAN | 13 | 11.136 | 23.338 | 26.692 | +0.06 | +0.25 | +0.424 | 4.594 |
| ATOM | 57 | C3 | MAN | 13 | 9.581 | 24.388 | 25.282 | −0.07 | +0.06 | +0.113 | 4.594 |
| ATOM | 58 | O3 | MAN | 13 | 10.496 | 23.832 | 24.349 | +0.09 | −0.32 | −0.537 | 4.594 |
| ATOM | 59 | HO3 | MAN | 13 | 10.282 | 22.834 | 24.210 | +0.08 | +0.25 | +0.424 | 4.594 |
| ATOM | 60 | C2 | MAN | 13 | 8.587 | 25.292 | 24.546 | −0.24 | +0.07 | +0.113 | 4.594 |
| ATOM | 61 | O2 | MAN | 13 | 9.286 | 26.234 | 23.747 | +0.20 | −0.36 | −0.537 | 4.594 |
| ATOM | 62 | HO2 | MAN | 13 | 10.197 | 25.840 | 23.469 | +0.11 | +0.28 | +0.424 | 4.594 |
| ATOM | 63 | C4 | MAN | 13 | 4.568 | 27.817 | 27.366 | −0.38 | +0.05 | +0.113 | 4.594 |
| ATOM | 64 | O4 | MAN | 13 | 5.675 | 28.519 | 27.911 | +0.04 | −0.11 | −0.537 | 4.594 |
| ATOM | 65 | HO4 | MAN | 13 | 5.877 | 28.164 | 28.857 | −0.23 | −0.16 | +0.424 | 4.594 |
| ATOM | 66 | C3 | MAN | 13 | 3.728 | 28.748 | 26.487 | −0.20 | +0.07 | +0.113 | 4.594 |
| ATOM | 67 | O3 | MAN | 13 | 3.321 | 29.961 | 27.179 | +0.15 | −0.12 | −0.227 | 4.594 |
| ATOM | 68 | C1 | BDG | 13 | 3.533 | 31.206 | 26.511 | −0.08 | +0.13 | +0.227 | 4.594 |
| ATOM | 69 | C2 | BDG | 13 | 3.905 | 32.297 | 27.542 | −0.10 | +0.06 | +0.113 | 4.594 |
| ATOM | 70 | O22 | BDG | 13 | 4.267 | 33.490 | 26.843 | +0.22 | −0.06 | −0.113 | 4.594 |
| ATOM | 71 | C3 | BDG | 13 | 2.710 | 32.614 | 28.472 | −0.12 | +0.05 | +0.113 | 4.594 |
| ATOM | 72 | O3 | BDG | 13 | 3.024 | 33.709 | 29.296 | +0.19 | −0.24 | −0.537 | 4.594 |
| ATOM | 73 | HO3 | BDG | 13 | 2.258 | 33.853 | 29.883 | +0.11 | +0.19 | +0.424 | 4.594 |
| ATOM | 74 | C4 | BDG | 13 | 1.409 | 32.911 | 27.682 | −0.04 | +0.06 | +0.113 | 4.594 |
| ATOM | 75 | O4 | BDG | 13 | 0.333 | 33.024 | 28.583 | +0.22 | −0.29 | −0.537 | 4.594 |
| ATOM | 76 | HO4 | BDG | 13 | −0.460 | 33.235 | 28.058 | +0.12 | +0.24 | +0.424 | 4.594 |
| ATOM | 77 | C5 | BDG | 13 | 1.136 | 31.800 | 26.624 | −0.04 | +0.07 | +0.113 | 4.594 |
| ATOM | 78 | C6 | BDG | 13 | −0.046 | 32.127 | 25.666 | −0.02 | +0.08 | +0.113 | 4.594 |
| ATOM | 79 | O6 | BDG | 13 | −1.275 | 32.160 | 26.359 | +0.22 | −0.38 | −0.537 | 4.594 |
| ATOM | 80 | H6 | BDG | 13 | −1.952 | 32.356 | 25.709 | +0.12 | +0.32 | +0.424 | 4.594 |
| ATOM | 81 | O | BDG | 13 | 2.315 | 31.584 | 25.809 | +0.23 | −0.14 | −0.227 | 4.594 |
| ATOM | 82 | C2 | MAN | 13 | 2.523 | 27.977 | 25.927 | −0.25 | +0.09 | +0.113 | 4.594 |
| ATOM | 83 | O2 | MAN | 13 | 1.693 | 27.550 | 26.997 | +0.00 | −0.49 | −0.537 | 4.594 |
| ATOM | 84 | HO2 | MAN | 13 | 0.777 | 27.258 | 26.627 | +0.09 | +0.45 | +0.424 | 4.594 |
| ATOM | 85 | C4 | MAN | 13 | 2.663 | 29.811 | 21.526 | −0.09 | +0.09 | +0.113 | 4.594 |
| ATOM | 86 | O4 | MAN | 13 | 3.030 | 30.578 | 22.665 | +0.22 | −0.39 | −0.537 | 4.594 |
| ATOM | 87 | HO4 | MAN | 13 | 3.444 | 29.959 | 23.376 | +0.12 | +0.31 | +0.424 | 4.594 |
| TER | | | | | | | | | | | |
| ENDMDL | | | | | | | | | | | |
| MODEL | 8 | | | | | | | | | | |
| USER | Run = 8 | | | | | | | | | | |
| USER | Cluster Rank = 14 | | | | | | | | | | |
| USER | Number of conformations in this cluster = 1 | | | | | | | | | | |
| USER | | | | | | | | | | | |
| USER | RMSD from reference structure = 3.514 A | | | | | | | | | | |
| USER | | | | | | | | | | | |
| USER | Estimated Free Energy of Binding = −4.37 kcal/mol [= (1) + (3)] | | | | | | | | | | |
| USER | Estimated Inhibition Constant, Ki = +6.26e−04 [Temperature = 298.15 K] | | | | | | | | | | |
| USER | | | | | | | | | | | |
| USER | Final Docked Energy = −1.75 kcal/mol [= (1) + (2)] | | | | | | | | | | |
| USER | | | | | | | | | | | |
| USER | (1) Final Intermolecular Energy = −9.35 kcal/mol | | | | | | | | | | |
| USER | (2) Final Internal Energy of Ligand = +7.60 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 0.576064 26.670510 22.402362 | | | | | | | | | | |
| USER | NEWDPF quat0 −0.768156 0.624230 0.142386 −57.621239 | | | | | | | | | | |
| USER | NEWDPF ndihe 16 | | | | | | | | | | |
| USER | NEWDPF dihe0 −84.39 0.17 4.20 124.41 41.37 91.95 −29.52 9.45 32.29 −126.75 | | | | | | | | | | |
| | 61.41 −105.77 77.80 93.71 25.51 −23.43 | | | | | | | | | | |
| USER | | | | | | | | | | | |
| USER | | | | Rank | x | y | z | vdW | Elec | q | RMS |
| ATOM | 1 | C5 | MAN | 14 | 5.366 | 22.605 | 19.400 | −0.29 | +0.12 | +0.113 | 3.514 |
| ATOM | 2 | HO4 | MAN | 14 | 4.592 | 21.936 | 21.873 | +0.06 | +0.76 | +0.424 | 3.514 |
| ATOM | 3 | O4 | MAN | 14 | 4.765 | 21.158 | 21.220 | −0.24 | −0.86 | −0.537 | 3.514 |
| ATOM | 4 | O5 | MAN | 14 | 4.870 | 23.220 | 18.188 | +0.12 | −0.22 | −0.227 | 3.514 |
| ATOM | 5 | HO2 | MAN | 14 | 1.910 | 22.087 | 16.862 | +0.05 | +0.17 | +0.424 | 3.514 |
| ATOM | 6 | O2 | MAN | 14 | 2.527 | 21.766 | 17.621 | +0.04 | −0.20 | −0.537 | 3.514 |
| ATOM | 7 | C2 | MAN | 14 | 2.556 | 22.738 | 18.648 | −0.46 | +0.05 | +0.113 | 3.514 |
| ATOM | 8 | C1 | MAN | 14 | 3.575 | 23.813 | 18.311 | −0.36 | +0.19 | +0.227 | 3.514 |
| ATOM | 9 | C4 | MAN | 14 | 4.379 | 21.543 | 19.913 | −0.41 | +0.12 | +0.113 | 3.514 |
| ATOM | 10 | O3 | MAN | 14 | 2.042 | 21.026 | 20.249 | +0.06 | −0.41 | −0.537 | 3.514 |
| ATOM | 11 | HO3 | MAN | 14 | 1.096 | 21.408 | 20.393 | −0.09 | +0.01 | +0.424 | 3.514 |
| ATOM | 12 | C3 | MAN | 14 | 2.942 | 22.084 | 19.966 | −0.51 | +0.08 | +0.113 | 3.514 |
| ATOM | 13 | C6 | MAN | 14 | 6.727 | 22.012 | 19.075 | −0.31 | +0.11 | +0.113 | 3.514 |
| ATOM | 14 | O6 | MAN | 14 | 7.596 | 22.987 | 18.511 | +0.07 | −0.53 | −0.537 | 3.514 |
| ATOM | 15 | H6 | MAN | 14 | 7.081 | 23.867 | 18.371 | +0.11 | +0.43 | +0.424 | 3.514 |
| ATOM | 16 | O3 | MAN | 14 | 3.537 | 24.776 | 19.315 | +0.05 | −0.19 | −0.227 | 3.514 |
| ATOM | 17 | C3 | MAN | 14 | 3.270 | 26.110 | 18.875 | −0.36 | +0.09 | +0.113 | 3.514 |
| ATOM | 18 | C2 | MAN | 14 | 4.561 | 26.911 | 19.014 | −0.21 | +0.10 | +0.113 | 3.514 |
| ATOM | 19 | O2 | MAN | 14 | 5.121 | 26.710 | 20.305 | +0.17 | −0.53 | −0.537 | 3.514 |
| ATOM | 20 | HO2 | MAN | 14 | 5.633 | 27.557 | 20.593 | +0.11 | +0.38 | +0.424 | 3.514 |
| ATOM | 21 | C1 | MAN | 14 | 4.286 | 28.382 | 18.803 | −0.24 | +0.19 | +0.227 | 3.514 |
| ATOM | 22 | O4 | NAG | 14 | 5.447 | 29.100 | 19.015 | +0.17 | −0.19 | −0.227 | 3.514 |
| ATOM | 23 | C4 | NAG | 14 | 6.088 | 29.678 | 17.871 | −0.21 | +0.11 | +0.113 | 3.514 |
| ATOM | 24 | C5 | NAG | 14 | 7.113 | 28.666 | 17.317 | −0.20 | +0.12 | +0.113 | 3.514 |
| ATOM | 25 | C6 | NAG | 14 | 6.493 | 27.519 | 16.535 | −0.34 | +0.13 | +0.113 | 3.514 |
| ATOM | 26 | O6 | NAG | 14 | 6.850 | 27.563 | 15.159 | −0.04 | −0.73 | −0.537 | 3.514 |
| ATOM | 27 | H6 | NAG | 14 | 7.769 | 28.017 | 15.058 | +0.08 | +0.63 | +0.424 | 3.514 |
| ATOM | 28 | O5 | NAG | 14 | 8.004 | 29.309 | 16.416 | +0.11 | −0.28 | −0.227 | 3.514 |
| ATOM | 29 | C1 | NAG | 14 | 8.752 | 30.331 | 17.043 | −0.16 | +0.13 | +0.113 | 3.514 |
| ATOM | 30 | C2 | NAG | 14 | 7.784 | 31.489 | 17.336 | −0.12 | +0.25 | +0.211 | 3.514 |
| ATOM | 31 | N2 | NAG | 14 | 8.470 | 32.622 | 17.914 | −0.07 | −0.64 | −0.650 | 3.514 |
| ATOM | 32 | HN2 | NAG | 14 | 8.483 | 33.523 | 17.416 | +0.11 | +0.46 | +0.440 | 3.514 |
| ATOM | 33 | C7 | NAG | 14 | 9.086 | 32.507 | 19.089 | −0.04 | +0.31 | +0.396 | 3.514 |
| ATOM | 34 | O7 | NAG | 14 | 9.107 | 31.459 | 19.733 | +0.23 | −0.30 | −0.396 | 3.514 |
| ATOM | 35 | C8 | NAG | 14 | 9.778 | 33.741 | 19.637 | −0.02 | +0.00 | +0.000 | 3.514 |
| ATOM | 36 | C3 | NAG | 14 | 6.728 | 30.993 | 18.317 | −0.15 | +0.10 | +0.113 | 3.514 |
| ATOM | 37 | O3 | NAG | 14 | 5.712 | 31.973 | 18.469 | +0.14 | −0.47 | −0.537 | 3.514 |
| ATOM | 38 | HO3 | NAG | 14 | 5.143 | 32.015 | 17.611 | +0.08 | +0.39 | +0.424 | 3.514 |
| ATOM | 39 | O5 | MAN | 14 | 3.276 | 28.823 | 19.722 | +0.13 | −0.18 | −0.227 | 3.514 |
| ATOM | 40 | C5 | MAN | 14 | 2.011 | 28.218 | 19.432 | −0.35 | +0.08 | +0.113 | 3.514 |
| ATOM | 41 | C6 | MAN | 14 | 0.919 | 29.017 | 20.158 | −0.34 | +0.08 | +0.113 | 3.514 |
| ATOM | 42 | O6 | MAN | 14 | 1.022 | 30.439 | 19.889 | +0.08 | −0.20 | −0.227 | 3.514 |
| ATOM | 43 | C1 | MAN | 14 | 0.723 | 31.282 | 20.984 | −0.13 | +0.19 | +0.227 | 3.514 |
| ATOM | 44 | O5 | MAN | 14 | −0.658 | 31.671 | 21.086 | +0.17 | −0.21 | −0.227 | 3.514 |
| ATOM | 45 | C5 | MAN | 14 | −1.568 | 30.637 | 21.541 | −0.21 | +0.12 | +0.113 | 3.514 |
| ATOM | 46 | C6 | MAN | 14 | −2.990 | 31.190 | 21.502 | +0.09 | +0.16 | +0.113 | 3.514 |
| ATOM | 47 | O6 | MAN | 14 | −3.361 | 31.538 | 20.153 | +0.11 | −0.27 | −0.227 | 3.514 |
| ATOM | 48 | C1 | MAN | 14 | −4.724 | 31.853 | 20.023 | −0.22 | +0.27 | +0.227 | 3.514 |
| ATOM | 49 | O5 | MAN | 14 | −4.981 | 32.746 | 18.936 | +0.15 | −0.23 | −0.227 | 3.514 |
| ATOM | 50 | C5 | MAN | 14 | −4.577 | 32.245 | 17.639 | −0.29 | +0.14 | +0.113 | 3.514 |
| ATOM | 51 | C6 | MAN | 14 | −4.929 | 33.306 | 16.617 | −0.34 | +0.13 | +0.113 | 3.514 |
| ATOM | 52 | O6 | MAN | 14 | −5.992 | 32.881 | 15.776 | −0.06 | −0.58 | −0.537 | 3.514 |
| ATOM | 53 | H6 | MAN | 14 | −5.892 | 31.874 | 15.579 | +0.06 | +0.49 | +0.424 | 3.514 |
| ATOM | 54 | C4 | MAN | 14 | −5.273 | 30.915 | 17.326 | −0.56 | +0.13 | +0.113 | 3.514 |
| ATOM | 55 | O4 | MAN | 14 | −4.679 | 30.360 | 16.165 | −0.16 | −0.63 | −0.537 | 3.514 |
| ATOM | 56 | HO4 | MAN | 14 | −4.587 | 29.341 | 16.279 | −0.06 | +0.43 | +0.424 | 3.514 |
| ATOM | 57 | C3 | MAN | 14 | −5.154 | 29.916 | 18.484 | −0.53 | +0.12 | +0.113 | 3.514 |
| ATOM | 58 | O3 | MAN | 14 | −6.004 | 28.805 | 18.242 | −0.16 | −0.54 | −0.537 | 3.514 |
| ATOM | 59 | HO3 | MAN | 14 | −5.472 | 27.931 | 18.371 | −0.26 | +0.19 | +0.424 | 3.514 |
| ATOM | 60 | C2 | MAN | 14 | −5.524 | 30.561 | 19.823 | −0.33 | +0.15 | +0.113 | 3.514 |
| ATOM | 61 | O2 | MAN | 14 | −6.916 | 30.842 | 19.858 | +0.07 | −0.67 | −0.537 | 3.514 |
| ATOM | 62 | HO2 | MAN | 14 | −7.416 | 30.176 | 19.252 | +0.09 | +0.51 | +0.424 | 3.514 |
| ATOM | 63 | C4 | MAN | 14 | −1.144 | 30.130 | 22.923 | −0.17 | +0.12 | +0.113 | 3.514 |
| ATOM | 64 | O4 | MAN | 14 | −2.042 | 29.123 | 23.368 | −0.22 | −0.94 | −0.537 | 3.514 |
| ATOM | 65 | HO4 | MAN | 14 | −2.745 | 29.541 | 23.995 | +0.16 | +0.98 | +0.424 | 3.514 |
| ATOM | 66 | C3 | MAN | 14 | 0.264 | 29.552 | 22.763 | −0.14 | +0.09 | +0.113 | 3.514 |
| ATOM | 67 | O3 | MAN | 14 | 0.740 | 28.885 | 23.965 | +0.18 | −0.18 | −0.227 | 3.514 |
| ATOM | 68 | C1 | BDG | 14 | 0.943 | 27.472 | 23.901 | −0.32 | +0.19 | +0.227 | 3.514 |
| ATOM | 69 | C2 | BDG | 14 | −0.155 | 26.748 | 24.715 | −0.49 | +0.10 | +0.113 | 3.514 |
| ATOM | 70 | O22 | BDG | 14 | −0.029 | 25.339 | 24.509 | −0.10 | −0.09 | −0.113 | 3.514 |
| ATOM | 71 | C3 | BDG | 14 | −0.010 | 27.040 | 26.227 | −0.47 | +0.12 | +0.113 | 3.514 |
| ATOM | 72 | O3 | BDG | 14 | −0.921 | 26.254 | 26.954 | −0.13 | −0.68 | −0.537 | 3.514 |
| ATOM | 73 | HO3 | BDG | 14 | −0.817 | 26.491 | 27.894 | +0.01 | +0.80 | +0.424 | 3.514 |
| ATOM | 74 | C4 | BDG | 14 | 1.429 | 26.783 | 26.744 | −0.50 | +0.12 | +0.113 | 3.514 |
| ATOM | 75 | O4 | BDG | 14 | 1.531 | 27.227 | 28.077 | −0.34 | −0.61 | −0.537 | 3.514 |
| ATOM | 76 | HO4 | BDG | 14 | 2.436 | 27.028 | 28.377 | +0.02 | +0.30 | +0.424 | 3.514 |
| ATOM | 77 | C5 | BDG | 14 | 2.479 | 27.495 | 25.839 | −0.32 | +0.10 | +0.113 | 3.514 |
| ATOM | 78 | C6 | BDG | 14 | 3.952 | 27.128 | 26.182 | −0.36 | +0.09 | +0.113 | 3.514 |
| ATOM | 79 | O6 | BDG | 14 | 4.316 | 27.606 | 27.460 | +0.01 | −0.24 | −0.537 | 3.514 |
| ATOM | 80 | H6 | BDG | 14 | 5.230 | 27.352 | 27.597 | +0.07 | +0.13 | +0.424 | 3.514 |
| ATOM | 81 | O | BDG | 14 | 2.255 | 27.153 | 24.448 | +0.05 | −0.24 | −0.227 | 3.514 |
| ATOM | 82 | C2 | MAN | 14 | 1.225 | 30.660 | 22.306 | −0.07 | +0.09 | +0.113 | 3.514 |
| ATOM | 83 | O2 | MAN | 14 | 1.297 | 31.669 | 23.303 | +0.23 | −0.38 | −0.537 | 3.514 |
| ATOM | 84 | HO2 | MAN | 14 | 2.109 | 32.276 | 23.121 | +0.12 | +0.29 | +0.424 | 3.514 |
| ATOM | 85 | C4 | MAN | 14 | 2.107 | 26.710 | 19.702 | −0.38 | +0.06 | +0.113 | 3.514 |
| ATOM | 86 | O4 | MAN | 14 | 0.887 | 26.083 | 19.330 | −0.05 | −0.25 | −0.537 | 3.514 |
| ATOM | 87 | HO4 | MAN | 14 | 0.219 | 26.796 | 19.005 | +0.18 | +0.26 | +0.424 | 3.514 |
| TER | | | | | | | | | | | |
| ENDMDL | | | | | | | | | | | |
| MODEL | 88 | | | | | | | | | | |
| USER | Run = 88 | | | | | | | | | | |
| USER | Cluster Rank = 15 | | | | | | | | | | |
| USER | Number of conformations in this cluster = 1 | | | | | | | | | | |
| USER | | | | | | | | | | | |
| USER | RMSD from reference structure = 4.209 A | | | | | | | | | | |
| USER | | | | | | | | | | | |
| USER | Estimated Free Energy of Binding = −2.37 kcal/mol [= (1) + (3)] | | | | | | | | | | |
| USER | Estimated Inhibition Constant, Ki = +0.02 [Temperature = 298.15 K] | | | | | | | | | | |
| USER | | | | | | | | | | | |
| USER | Final Docked Energy = −1.75 kcal/mol [= (1) + (2)] | | | | | | | | | | |
| USER | | | | | | | | | | | |
| USER | (1) Final Intermolecular Energy = −7.35 kcal/mol | | | | | | | | | | |
| USER | (2) Final Internal Energy of Ligand = +5.60 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 2.732461 31.342615 23.008132 | | | | | | | | | | |
| USER | NEWDPF quat0 −0.815776 0.577094 0.038369 73.064788 | | | | | | | | | | |
| USER | NEWDPF ndihe 16 | | | | | | | | | | |
| USER | NEWDPF dihe0 −23.72 112.74 −155.26 −4.74 39.56 116.70 4.83 135.75 27.70 | | | | | | | | | | |
| | −73.11 16.25 113.48 −34.80 −39.86 36.54 33.16 | | | | | | | | | | |
| USER | | | | | | | | | | | |
| USER | | | | Rank | x | y | z | vdW | Elec | q | RMS |
| ATOM | 1 | C5 | MAN | 15 | 6.666 | 25.774 | 24.424 | −0.28 | +0.11 | +0.113 | 4.209 |
| ATOM | 2 | HO4 | MAN | 15 | 7.945 | 27.984 | 23.619 | +0.12 | +0.30 | +0.424 | 4.209 |
| ATOM | 3 | O4 | MAN | 15 | 8.407 | 27.427 | 24.354 | +0.21 | −0.36 | −0.537 | 4.209 |
| ATOM | 4 | O5 | MAN | 15 | 5.490 | 25.351 | 25.152 | −0.21 | −0.30 | −0.227 | 4.209 |
| ATOM | 5 | HO2 | MAN | 15 | 4.892 | 26.718 | 28.246 | −0.13 | +0.05 | +0.424 | 4.209 |
| ATOM | 6 | O2 | MAN | 15 | 5.681 | 26.752 | 27.586 | −0.06 | −0.17 | −0.537 | 4.209 |
| ATOM | 7 | C2 | MAN | 15 | 5.273 | 27.400 | 26.397 | −0.30 | +0.07 | +0.113 | 4.209 |
| ATOM | 8 | C1 | MAN | 15 | 4.583 | 26.406 | 25.479 | −0.31 | +0.26 | +0.227 | 4.209 |
| ATOM | 9 | C4 | MAN | 15 | 7.426 | 26.860 | 25.204 | −0.18 | +0.08 | +0.113 | 4.209 |
| ATOM | 10 | O3 | MAN | 15 | 7.182 | 28.845 | 26.560 | +0.16 | −0.23 | −0.537 | 4.209 |
| ATOM | 11 | HO3 | MAN | 15 | 6.585 | 29.652 | 26.791 | +0.11 | +0.18 | +0.424 | 4.209 |
| ATOM | 12 | C3 | MAN | 15 | 6.486 | 27.976 | 25.682 | −0.15 | +0.07 | +0.113 | 4.209 |
| ATOM | 13 | C6 | MAN | 15 | 7.516 | 24.536 | 24.189 | −0.34 | +0.08 | +0.113 | 4.209 |
| ATOM | 14 | O6 | MAN | 15 | 8.536 | 24.780 | 23.229 | +0.14 | −0.40 | −0.537 | 4.209 |
| ATOM | 15 | H6 | MAN | 15 | 9.105 | 25.583 | 23.530 | +0.11 | +0.29 | +0.424 | 4.209 |
| ATOM | 16 | O3 | MAN | 15 | 4.150 | 27.090 | 24.346 | −0.10 | −0.30 | −0.227 | 4.209 |
| ATOM | 17 | C3 | MAN | 15 | 3.118 | 28.054 | 24.569 | −0.19 | +0.10 | +0.113 | 4.209 |
| ATOM | 18 | C2 | MAN | 15 | 1.787 | 27.396 | 24.222 | −0.31 | +0.11 | +0.113 | 4.209 |
| ATOM | 19 | O2 | MAN | 15 | 1.874 | 26.761 | 22.953 | +0.06 | −0.45 | −0.537 | 4.209 |
| ATOM | 20 | HO2 | MAN | 15 | 0.950 | 26.776 | 22.498 | −0.16 | +0.16 | +0.424 | 4.209 |
| ATOM | 21 | C1 | MAN | 15 | 0.686 | 28.431 | 24.197 | −0.20 | +0.19 | +0.227 | 4.209 |
| ATOM | 22 | O4 | NAG | 15 | −0.495 | 27.834 | 23.800 | +0.06 | −0.20 | −0.227 | 4.209 |
| ATOM | 23 | C4 | NAG | 15 | −1.717 | 28.530 | 24.077 | −0.31 | +0.15 | +0.113 | 4.209 |
| ATOM | 24 | C5 | NAG | 15 | −1.617 | 29.953 | 23.487 | −0.13 | +0.15 | +0.113 | 4.209 |
| ATOM | 25 | C6 | NAG | 15 | −1.790 | 30.011 | 21.978 | −0.15 | +0.14 | +0.113 | 4.209 |
| ATOM | 26 | O6 | NAG | 15 | −2.976 | 30.703 | 21.607 | +0.39 | −0.83 | −0.537 | 4.209 |
| ATOM | 27 | H6 | NAG | 15 | −3.208 | 31.398 | 22.331 | +0.10 | +0.64 | +0.424 | 4.209 |
| ATOM | 28 | O5 | NAG | 15 | −2.639 | 30.782 | 24.024 | +0.22 | −0.31 | −0.227 | 4.209 |
| ATOM | 29 | C1 | NAG | 15 | −2.529 | 30.923 | 25.426 | −0.15 | +0.11 | +0.113 | 4.209 |
| ATOM | 30 | C2 | NAG | 15 | −2.922 | 29.575 | 26.051 | −0.27 | +0.24 | +0.211 | 4.209 |
| ATOM | 31 | N2 | NAG | 15 | −2.914 | 29.636 | 27.495 | −0.16 | −0.65 | −0.650 | 4.209 |
| ATOM | 32 | HN2 | NAG | 15 | −2.500 | 30.450 | 27.972 | −0.11 | +0.37 | +0.440 | 4.209 |
| ATOM | 33 | C7 | NAG | 15 | −3.440 | 28.647 | 28.213 | −0.40 | +0.48 | +0.396 | 4.209 |
| ATOM | 34 | O7 | NAG | 15 | −3.949 | 27.649 | 27.705 | +0.07 | −0.56 | −0.396 | 4.209 |
| ATOM | 35 | C8 | NAG | 15 | −3.402 | 28.785 | 29.724 | −0.44 | +0.00 | +0.000 | 4.209 |
| ATOM | 36 | C3 | NAG | 15 | −1.917 | 28.525 | 25.593 | −0.35 | +0.14 | +0.113 | 4.209 |
| ATOM | 37 | O3 | NAG | 15 | −2.349 | 27.234 | 25.997 | −0.19 | −0.68 | −0.537 | 4.209 |
| ATOM | 38 | HO3 | NAG | 15 | −3.163 | 26.951 | 25.433 | +0.05 | +0.73 | +0.424 | 4.209 |
| ATOM | 39 | O5 | MAN | 15 | 1.026 | 29.479 | 23.278 | +0.19 | −0.18 | −0.227 | 4.209 |
| ATOM | 40 | C5 | MAN | 15 | 2.156 | 30.227 | 23.738 | −0.04 | +0.08 | +0.113 | 4.209 |
| ATOM | 41 | C6 | MAN | 15 | 2.212 | 31.545 | 22.953 | −0.02 | +0.08 | +0.113 | 4.209 |
| ATOM | 42 | O6 | MAN | 15 | 2.855 | 31.379 | 21.663 | +0.22 | −0.16 | −0.227 | 4.209 |
| ATOM | 43 | C1 | MAN | 15 | 1.981 | 31.366 | 20.552 | −0.13 | +0.18 | +0.227 | 4.209 |
| ATOM | 44 | O5 | MAN | 15 | 2.341 | 32.270 | 19.492 | +0.13 | −0.20 | −0.227 | 4.209 |
| ATOM | 45 | C5 | MAN | 15 | 3.482 | 31.873 | 18.690 | −0.27 | +0.10 | +0.113 | 4.209 |
| ATOM | 46 | C6 | MAN | 15 | 3.778 | 32.980 | 17.682 | −0.34 | +0.10 | +0.113 | 4.209 |
| ATOM | 47 | O6 | MAN | 15 | 3.128 | 34.206 | 18.073 | +0.02 | −0.18 | −0.227 | 4.209 |
| ATOM | 48 | C1 | MAN | 15 | 3.605 | 35.327 | 17.374 | −0.35 | +0.13 | +0.227 | 4.209 |
| ATOM | 49 | O5 | MAN | 15 | 2.923 | 36.532 | 17.732 | +0.05 | −0.08 | −0.227 | 4.209 |
| ATOM | 50 | C5 | MAN | 15 | 3.022 | 36.883 | 19.133 | −0.21 | +0.05 | +0.113 | 4.209 |
| ATOM | 51 | C6 | MAN | 15 | 2.256 | 38.176 | 19.328 | −0.16 | +0.04 | +0.113 | 4.209 |
| ATOM | 52 | O6 | MAN | 15 | 0.892 | 37.927 | 19.635 | +0.13 | −0.07 | −0.537 | 4.209 |
| ATOM | 53 | H6 | MAN | 15 | 0.560 | 37.115 | 19.096 | −0.18 | −0.17 | +0.424 | 4.209 |
| ATOM | 54 | C4 | MAN | 15 | 4.487 | 37.032 | 19.558 | −0.08 | +0.06 | +0.113 | 4.209 |
| ATOM | 55 | O4 | MAN | 15 | 4.536 | 37.150 | 20.970 | +0.22 | −0.28 | −0.537 | 4.209 |
| ATOM | 56 | HO4 | MAN | 15 | 5.342 | 36.620 | 21.331 | +0.12 | +0.23 | +0.424 | 4.209 |
| ATOM | 57 | C3 | MAN | 15 | 5.338 | 35.835 | 19.119 | −0.08 | +0.07 | +0.113 | 4.209 |
| ATOM | 58 | O3 | MAN | 15 | 6.713 | 36.127 | 19.325 | +0.22 | −0.35 | −0.537 | 4.209 |
| ATOM | 59 | HO3 | MAN | 15 | 7.157 | 35.350 | 19.836 | +0.12 | +0.28 | +0.424 | 4.209 |
| ATOM | 60 | C2 | MAN | 15 | 5.105 | 35.490 | 17.644 | −0.24 | +0.08 | +0.113 | 4.209 |
| ATOM | 61 | O2 | MAN | 15 | 5.640 | 36.513 | 16.816 | +0.13 | −0.39 | −0.537 | 4.209 |
| ATOM | 62 | HO2 | MAN | 15 | 6.423 | 36.973 | 17.301 | +0.11 | +0.30 | +0.424 | 4.209 |
| ATOM | 63 | C4 | MAN | 15 | 3.226 | 30.507 | 18.044 | −0.40 | +0.10 | +0.113 | 4.209 |
| ATOM | 64 | O4 | MAN | 15 | 4.342 | 30.126 | 17.254 | +0.01 | −0.46 | −0.537 | 4.209 |
| ATOM | 65 | HO4 | MAN | 15 | 4.165 | 30.364 | 16.268 | +0.13 | +0.32 | +0.424 | 4.209 |
| ATOM | 66 | C3 | MAN | 15 | 3.022 | 29.513 | 19.190 | −0.27 | +0.09 | +0.113 | 4.209 |
| ATOM | 67 | O3 | MAN | 15 | 2.924 | 28.136 | 18.732 | +0.03 | −0.17 | −0.227 | 4.209 |
| ATOM | 68 | C1 | BDG | 15 | 3.418 | 27.114 | 19.600 | −0.29 | +0.18 | +0.227 | 4.209 |
| ATOM | 69 | C2 | BDG | 15 | 4.705 | 26.499 | 19.005 | −0.21 | +0.10 | +0.113 | 4.209 |
| ATOM | 70 | O22 | BDG | 15 | 5.269 | 25.595 | 19.958 | +0.14 | −0.12 | −0.113 | 4.209 |
| ATOM | 71 | C3 | BDG | 15 | 4.400 | 25.718 | 17.705 | −0.35 | +0.12 | +0.113 | 4.209 |
| ATOM | 72 | O3 | BDG | 15 | 5.555 | 25.045 | 17.270 | +0.01 | −0.67 | −0.537 | 4.209 |
| ATOM | 73 | HO3 | BDG | 15 | 5.325 | 24.593 | 16.436 | +0.02 | +0.80 | +0.424 | 4.209 |
| ATOM | 74 | C4 | BDG | 15 | 3.241 | 24.702 | 17.884 | −0.43 | +0.10 | +0.113 | 4.209 |
| ATOM | 75 | O4 | BDG | 15 | 2.906 | 24.153 | 16.632 | +0.02 | −0.56 | −0.537 | 4.209 |
| ATOM | 76 | HO4 | BDG | 15 | 2.196 | 23.503 | 16.784 | +0.07 | +0.35 | +0.424 | 4.209 |
| ATOM | 77 | C5 | BDG | 15 | 2.004 | 25.382 | 18.543 | −0.33 | +0.08 | +0.113 | 4.209 |
| ATOM | 78 | C6 | BDG | 15 | 0.873 | 24.385 | 18.928 | −0.56 | +0.04 | +0.113 | 4.209 |
| ATOM | 79 | O6 | BDG | 15 | 0.292 | 23.800 | 17.782 | −0.13 | −0.20 | −0.537 | 4.209 |
| ATOM | 80 | H6 | BDG | 15 | −0.396 | 23.209 | 18.094 | −0.24 | −0.21 | +0.424 | 4.209 |
| ATOM | 81 | O | BDG | 15 | 2.400 | 26.083 | 19.749 | +0.03 | −0.12 | −0.227 | 4.209 |
| ATOM | 82 | C2 | MAN | 15 | 1.796 | 29.929 | 20.018 | −0.24 | +0.09 | +0.113 | 4.209 |
| ATOM | 83 | O2 | MAN | 15 | 0.633 | 29.875 | 19.205 | +0.03 | −0.45 | −0.537 | 4.209 |
| ATOM | 84 | HO2 | MAN | 15 | −0.206 | 29.908 | 19.802 | +0.08 | +0.38 | +0.424 | 4.209 |
| ATOM | 85 | C4 | MAN | 15 | 3.394 | 29.320 | 23.722 | −0.07 | +0.09 | +0.113 | 4.209 |
| ATOM | 86 | O4 | MAN | 15 | 4.504 | 30.022 | 24.265 | +0.22 | −0.36 | −0.537 | 4.209 |
| ATOM | 87 | HO4 | MAN | 15 | 4.220 | 30.983 | 24.504 | +0.12 | +0.27 | +0.424 | 4.209 |
| TER | | | | | | | | | | | |
| ENDMDL | | | | | | | | | | | |
| MODEL | 58 | | | | | | | | | | |
| USER | Run = 58 | | | | | | | | | | |
| USER | Cluster Rank = 16 | | | | | | | | | | |
| USER | Number of conformations in this cluster = 1 | | | | | | | | | | |
| USER | | | | | | | | | | | |
| USER | RMSD from reference structure = 3.787 A | | | | | | | | | | |
| USER | | | | | | | | | | | |
| USER | Estimated Free Energy of Binding = −3.38 kcal/mol [= (1) + (3)] | | | | | | | | | | |
| USER | Estimated Inhibition Constant, Ki = +0.00 [Temperature = 298.15 K] | | | | | | | | | | |
| USER | | | | | | | | | | | |
| USER | Final Docked Energy = −1.68 kcal/mol [= (1) + (2)] | | | | | | | | | | |
| USER | | | | | | | | | | | |
| USER | (1) Final Intermolecular Energy = −8.36 kcal/mol | | | | | | | | | | |
| USER | (2) Final Internal Energy of Ligand = +6.69 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 2.722859 27.133442 23.362630 | | | | | | | | | | |
| USER | NEWDPF quat0 −0.819265 0.093724 0.565704 −55.508895 | | | | | | | | | | |
| USER | NEWDPF ndihe 16 | | | | | | | | | | |
| USER | NEWDPF dihe0 0.64 93.30 −179.73 −31.22 −24.02 106.56 144.05 −73.89 | | | | | | | | | | |
| | 113.03 −101.20 −176.61 −178.15 −2.23 −152.26 34.42 −70.96 | | | | | | | | | | |
| USER | | | | | | | | | | | |
| USER | | | | Rank | x | y | z | vdW | Elec | q | RMS |
| ATOM | 1 | C5 | MAN | 16 | 4.185 | 23.354 | 17.699 | −0.33 | +0.11 | +0.113 | 3.787 |
| ATOM | 2 | HO4 | MAN | 16 | 4.807 | 22.273 | 20.068 | +0.10 | +0.49 | +0.424 | 3.787 |
| ATOM | 3 | O4 | MAN | 16 | 4.441 | 21.630 | 19.351 | +0.08 | −0.52 | −0.537 | 3.787 |
| ATOM | 4 | O5 | MAN | 16 | 3.223 | 24.240 | 17.080 | −0.03 | −0.23 | −0.227 | 3.787 |
| ATOM | 5 | HO2 | MAN | 16 | −0.122 | 23.653 | 17.594 | −0.23 | +0.14 | +0.424 | 3.787 |
| ATOM | 6 | O2 | MAN | 16 | 0.740 | 23.136 | 17.812 | +0.18 | −0.02 | −0.537 | 3.787 |
| ATOM | 7 | C2 | MAN | 16 | 1.514 | 23.877 | 18.735 | −0.60 | +0.04 | +0.113 | 3.787 |
| ATOM | 8 | C1 | MAN | 16 | 2.352 | 24.905 | 17.996 | −0.42 | +0.18 | +0.227 | 3.787 |
| ATOM | 9 | C4 | MAN | 16 | 3.473 | 22.301 | 18.564 | −0.35 | +0.08 | +0.113 | 3.787 |
| ATOM | 10 | O3 | MAN | 16 | 1.669 | 21.929 | 20.129 | −0.10 | −0.05 | −0.537 | 3.787 |
| ATOM | 11 | HO3 | MAN | 16 | 1.054 | 22.354 | 20.837 | +0.19 | −0.34 | +0.424 | 3.787 |
| ATOM | 12 | O3 | MAN | 16 | 2.438 | 22.941 | 19.501 | −0.45 | +0.05 | +0.113 | 3.787 |
| ATOM | 13 | C6 | MAN | 16 | 4.997 | 22.725 | 16.581 | −0.23 | +0.12 | +0.113 | 3.787 |
| ATOM | 14 | O6 | MAN | 16 | 6.337 | 22.475 | 16.989 | +0.05 | −0.55 | −0.537 | 3.787 |
| ATOM | 15 | H6 | MAN | 16 | 6.372 | 21.608 | 17.542 | +0.09 | +0.41 | +0.424 | 3.787 |
| ATOM | 16 | O3 | MAN | 16 | 3.057 | 25.648 | 18.939 | +0.02 | −0.17 | −0.227 | 3.787 |
| ATOM | 17 | C3 | MAN | 16 | 3.738 | 26.797 | 18.429 | −0.33 | +0.10 | +0.113 | 3.787 |
| ATOM | 18 | C2 | MAN | 16 | 5.206 | 26.423 | 18.248 | −0.22 | +0.11 | +0.113 | 3.787 |
| ATOM | 19 | O2 | MAN | 16 | 5.697 | 25.797 | 19.426 | +0.18 | −0.54 | −0.537 | 3.787 |
| ATOM | 20 | HO2 | MAN | 16 | 6.701 | 26.001 | 19.530 | +0.11 | +0.40 | +0.424 | 3.787 |
| ATOM | 21 | C1 | MAN | 16 | 6.025 | 27.658 | 17.951 | −0.20 | +0.22 | +0.227 | 3.787 |
| ATOM | 22 | O4 | NAG | 16 | 7.360 | 27.314 | 17.872 | +0.18 | −0.22 | −0.227 | 3.787 |
| ATOM | 23 | C4 | NAG | 16 | 8.118 | 27.841 | 16.776 | −0.21 | +0.13 | +0.113 | 3.787 |
| ATOM | 24 | C5 | NAG | 16 | 9.317 | 26.906 | 16.512 | −0.23 | +0.13 | +0.113 | 3.787 |
| ATOM | 25 | C6 | NAG | 16 | 8.953 | 25.618 | 15.792 | −0.43 | +0.15 | +0.113 | 3.787 |
| ATOM | 26 | O6 | NAG | 16 | 9.524 | 25.560 | 14.491 | −0.25 | −1.00 | −0.537 | 3.787 |
| ATOM | 27 | H6 | NAG | 16 | 10.397 | 26.108 | 14.475 | +0.08 | +0.72 | +0.424 | 3.787 |
| ATOM | 28 | O5 | NAG | 16 | 10.274 | 27.554 | 15.685 | +0.10 | −0.30 | −0.227 | 3.787 |
| ATOM | 29 | C1 | NAG | 16 | 10.801 | 28.718 | 16.287 | −0.15 | +0.12 | +0.113 | 3.787 |
| ATOM | 30 | C2 | NAG | 16 | 9.686 | 29.776 | 16.286 | −0.18 | +0.25 | +0.211 | 3.787 |
| ATOM | 31 | N2 | NAG | 16 | 10.150 | 31.037 | 16.819 | −0.06 | −0.70 | −0.650 | 3.787 |
| ATOM | 32 | HN2 | NAG | 16 | 10.804 | 31.049 | 17.613 | +0.12 | +0.39 | +0.440 | 3.787 |
| ATOM | 33 | C7 | NAG | 16 | 9.736 | 32.185 | 16.286 | −0.18 | +0.53 | +0.396 | 3.787 |
| ATOM | 34 | O7 | NAG | 16 | 8.956 | 32.238 | 15.336 | −0.16 | −0.89 | −0.396 | 3.787 |
| ATOM | 35 | C8 | NAG | 16 | 10.271 | 33.469 | 16.893 | −0.10 | +0.00 | +0.000 | 3.787 |
| ATOM | 36 | C3 | NAG | 16 | 8.540 | 29.263 | 17.150 | −0.16 | +0.12 | +0.113 | 3.787 |
| ATOM | 37 | O3 | NAG | 16 | 7.419 | 30.125 | 17.025 | +0.13 | −0.62 | −0.537 | 3.787 |
| ATOM | 38 | HO3 | NAG | 16 | 6.997 | 30.008 | 16.092 | +0.12 | +0.61 | +0.424 | 3.787 |
| ATOM | 39 | O5 | MAN | 16 | 5.839 | 28.626 | 18.994 | +0.18 | −0.20 | −0.227 | 3.787 |
| ATOM | 40 | C5 | MAN | 16 | 4.499 | 29.128 | 19.005 | −0.20 | +0.09 | +0.113 | 3.787 |
| ATOM | 41 | C6 | MAN | 16 | 4.474 | 30.415 | 19.839 | −0.11 | +0.09 | +0.113 | 3.787 |
| ATOM | 42 | O6 | MAN | 16 | 3.258 | 31.177 | 19.623 | +0.16 | −0.18 | −0.227 | 3.787 |
| ATOM | 43 | C1 | MAN | 16 | 2.670 | 31.716 | 20.789 | −0.09 | +0.17 | +0.227 | 3.787 |
| ATOM | 44 | O5 | MAN | 16 | 1.298 | 32.123 | 20.639 | +0.18 | −0.19 | −0.227 | 3.787 |
| ATOM | 45 | C5 | MAN | 16 | 0.329 | 31.046 | 20.562 | −0.20 | +0.10 | +0.113 | 3.787 |
| ATOM | 46 | C6 | MAN | 16 | −1.050 | 31.650 | 20.314 | −0.24 | +0.12 | +0.113 | 3.787 |
| ATOM | 47 | O6 | MAN | 16 | −2.061 | 30.623 | 20.323 | +0.08 | −0.25 | −0.227 | 3.787 |
| ATOM | 48 | C1 | MAN | 16 | −2.589 | 30.360 | 19.048 | −0.24 | +0.27 | +0.227 | 3.787 |
| ATOM | 49 | O5 | MAN | 16 | −3.376 | 29.166 | 19.012 | −0.09 | −0.23 | −0.227 | 3.787 |
| ATOM | 50 | C5 | MAN | 16 | −4.515 | 29.173 | 19.905 | −0.52 | +0.13 | +0.113 | 3.787 |
| ATOM | 51 | C6 | MAN | 16 | −5.230 | 27.849 | 19.730 | −0.71 | +0.10 | +0.113 | 3.787 |
| ATOM | 52 | O6 | MAN | 16 | −4.421 | 26.763 | 20.159 | −0.18 | −0.37 | −0.537 | 3.787 |
| ATOM | 53 | H6 | MAN | 16 | −3.438 | 26.947 | 19.907 | −0.15 | +0.25 | +0.424 | 3.787 |
| ATOM | 54 | C4 | MAN | 16 | −5.443 | 30.354 | 19.599 | −0.38 | +0.14 | +0.113 | 3.787 |
| ATOM | 55 | O4 | MAN | 16 | −6.410 | 30.445 | 20.632 | −0.08 | −0.87 | −0.537 | 3.787 |
| ATOM | 56 | HO4 | MAN | 16 | −6.577 | 31.436 | 20.859 | +0.09 | +0.64 | +0.424 | 3.787 |
| ATOM | 57 | C3 | MAN | 16 | −4.677 | 31.678 | 19.502 | −0.26 | +0.13 | +0.113 | 3.787 |
| ATOM | 58 | O3 | MAN | 16 | −5.535 | 32.688 | 18.990 | +0.16 | −0.54 | −0.537 | 3.787 |
| ATOM | 59 | HO3 | MAN | 16 | −5.484 | 33.521 | 19.595 | +0.11 | +0.38 | +0.424 | 3.787 |
| ATOM | 60 | C2 | MAN | 16 | −3.445 | 31.550 | 18.600 | −0.37 | +0.14 | +0.113 | 3.787 |
| ATOM | 61 | O2 | MAN | 16 | −3.847 | 31.378 | 17.250 | −0.03 | −0.73 | −0.537 | 3.787 |
| ATOM | 62 | HO2 | MAN | 16 | −4.764 | 31.825 | 17.105 | +0.08 | +0.52 | +0.424 | 3.787 |
| ATOM | 63 | C4 | MAN | 16 | 0.402 | 30.178 | 21.822 | −0.13 | +0.09 | +0.113 | 3.787 |
| ATOM | 64 | O4 | MAN | 16 | −0.553 | 29.129 | 21.746 | +0.11 | −0.48 | −0.537 | 3.787 |
| ATOM | 65 | HO4 | MAN | 16 | −1.402 | 29.398 | 22.263 | +0.10 | +0.50 | +0.424 | 3.787 |
| ATOM | 66 | C3 | MAN | 16 | 1.820 | 29.602 | 21.876 | −0.11 | +0.09 | +0.113 | 3.787 |
| ATOM | 67 | O3 | MAN | 16 | 1.992 | 28.625 | 22.939 | +0.16 | −0.18 | −0.227 | 3.787 |
| ATOM | 68 | C1 | BDG | 16 | 2.041 | 29.111 | 24.282 | −0.10 | +0.18 | +0.227 | 3.787 |
| ATOM | 69 | C2 | BDG | 16 | 2.389 | 27.953 | 25.246 | −0.23 | +0.10 | +0.113 | 3.787 |
| ATOM | 70 | O22 | BDG | 16 | 2.589 | 28.487 | 26.557 | +0.14 | −0.08 | −0.113 | 3.787 |
| ATOM | 71 | C3 | BDG | 16 | 1.243 | 26.916 | 25.305 | −0.43 | +0.11 | +0.113 | 3.787 |
| ATOM | 72 | O3 | BDG | 16 | 1.517 | 25.959 | 26.297 | −0.13 | −0.62 | −0.537 | 3.787 |
| ATOM | 73 | HO3 | BDG | 16 | 0.788 | 25.311 | 26.279 | +0.03 | +0.53 | +0.424 | 3.787 |
| ATOM | 74 | C4 | BDG | 16 | −0.135 | 27.572 | 25.581 | −0.35 | +0.11 | +0.113 | 3.787 |
| ATOM | 75 | O4 | BDG | 16 | −1.146 | 26.601 | 25.455 | −0.08 | −0.52 | −0.537 | 3.787 |
| ATOM | 76 | HO4 | BDG | 16 | −1.992 | 27.039 | 25.659 | +0.07 | +0.49 | +0.424 | 3.787 |
| ATOM | 77 | C5 | BDG | 16 | −0.385 | 28.766 | 24.611 | −0.20 | +0.10 | +0.113 | 3.787 |
| ATOM | 78 | C6 | BDG | 16 | −1.660 | 29.591 | 24.950 | −0.21 | +0.13 | +0.113 | 3.787 |
| ATOM | 79 | O6 | BDG | 16 | −2.832 | 28.828 | 24.761 | −0.01 | −0.99 | −0.537 | 3.787 |
| ATOM | 80 | H6 | BDG | 16 | −3.570 | 29.399 | 24.979 | +0.09 | +0.71 | +0.424 | 3.787 |
| ATOM | 81 | O | BDG | 16 | 0.745 | 29.674 | 24.633 | +0.20 | −0.18 | −0.227 | 3.787 |
| ATOM | 82 | C2 | MAN | 16 | 2.836 | 30.749 | 21.983 | −0.05 | +0.08 | +0.113 | 3.787 |
| ATOM | 83 | O2 | MAN | 16 | 2.626 | 31.459 | 23.195 | +0.23 | −0.37 | −0.537 | 3.787 |
| ATOM | 84 | HO2 | MAN | 16 | 3.442 | 32.057 | 23.390 | +0.12 | +0.27 | +0.424 | 3.787 |
| ATOM | 85 | C4 | MAN | 16 | 3.544 | 27.991 | 19.396 | −0.26 | +0.09 | +0.113 | 3.787 |
| ATOM | 86 | O4 | MAN | 16 | 2.202 | 28.450 | 19.320 | +0.02 | −0.38 | −0.537 | 3.787 |
| ATOM | 87 | HO4 | MAN | 16 | 2.194 | 29.446 | 19.057 | +0.08 | +0.32 | +0.424 | 3.787 |
| TER | | | | | | | | | | | |
| ENDMDL | | | | | | | | | | | |
| MODEL | 34 | | | | | | | | | | |
| USER | Run = 34 | | | | | | | | | | |
| USER | Cluster Rank = 17 | | | | | | | | | | |
| USER | Number of conformations in this cluster = 1 | | | | | | | | | | |
| USER | | | | | | | | | | | |
| USER | RMSD from reference structure = 3.802 A | | | | | | | | | | |
| USER | | | | | | | | | | | |
| USER | Estimated Free Energy of Binding = +2.25 kcal/mol [= (1) + (3)] | | | | | | | | | | |
| USER | Estimated Inhibition Constant, Ki = +44.25 [Temperature = 298.15 K] | | | | | | | | | | |
| USER | | | | | | | | | | | |
| USER | Final Docked Energy = −1.66 kcal/mol [= (1) + (2)] | | | | | | | | | | |
| USER | | | | | | | | | | | |
| USER | (1) Final Intermolecular Energy = −2.74 kcal/mol | | | | | | | | | | |
| USER | (2) Final Internal Energy of Ligand = +1.07 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 1.594402 26.475761 23.221122 | | | | | | | | | | |
| USER | NEWDPF quat0 −0.988132 0.030134 0.150624 −44.250026 | | | | | | | | | | |
| USER | NEWDPF ndihe 16 | | | | | | | | | | |
| USER | NEWDPF dihe0 120.90 61.79 −179.98 −10.77 2.30 36.26 25.09 −17.08 31.77 | | | | | | | | | | |
| | −16.96 −16.15 82.83 67.17 100.89 23.62 −37.06 | | | | | | | | | | |
| USER | | | | | | | | | | | |
| USER | | | | Rank | x | y | z | vdW | Elec | q | RMS |
| ATOM | 1 | C5 | MAN | 17 | 5.004 | 23.447 | 17.958 | −0.28 | +0.12 | +0.113 | 3.802 |
| ATOM | 2 | HO4 | MAN | 17 | 5.767 | 22.780 | 20.436 | +0.10 | +0.52 | +0.424 | 3.802 |
| ATOM | 3 | O4 | MAN | 17 | 5.762 | 22.023 | 19.737 | +0.06 | −0.57 | −0.537 | 3.802 |
| ATOM | 4 | O5 | MAN | 17 | 3.836 | 23.850 | 17.206 | +0.00 | −0.24 | −0.227 | 3.802 |
| ATOM | 5 | HO2 | MAN | 17 | 0.971 | 21.976 | 17.490 | −0.31 | −0.21 | +0.424 | 3.802 |
| ATOM | 6 | O2 | MAN | 17 | 1.942 | 21.858 | 17.811 | +0.10 | −0.03 | −0.537 | 3.802 |
| ATOM | 7 | C2 | MAN | 17 | 2.258 | 22.875 | 18.741 | −0.50 | +00.4 | +0.113 | 3.802 |
| ATOM | 8 | C1 | MAN | 17 | 2.684 | 24.132 | 18.004 | −0.48 | +0.17 | +0.227 | 3.802 |
| ATOM | 9 | C4 | MAN | 17 | 4.690 | 22.222 | 18.832 | −0.28 | +0.11 | +0.113 | 3.802 |
| ATOM | 10 | O3 | MAN | 17 | 3.039 | 21.198 | 20.270 | −0.21 | −0.52 | −0.537 | 3.802 |
| ATOM | 11 | HO3 | MAN | 17 | 2.237 | 21.358 | 20.896 | −0.06 | +0.42 | +0.424 | 3.802 |
| ATOM | 12 | C3 | MAN | 17 | 3.397 | 22.416 | 19.638 | −0.44 | +0.09 | +0.113 | 3.602 |
| ATOM | 13 | C6 | MAN | 17 | 6.108 | 23.168 | 16.953 | −0.34 | +0.13 | +0.113 | 3.802 |
| ATOM | 14 | O6 | MAN | 17 | 7.193 | 24.075 | 17.107 | +0.07 | −0.63 | −0.537 | 3.602 |
| ATOM | 15 | H6 | MAN | 17 | 7.746 | 24.098 | 16.240 | +0.09 | +0.54 | +0.424 | 3.802 |
| ATOM | 16 | O3 | MAN | 17 | 2.935 | 25.124 | 18.948 | +0.01 | −0.16 | −0.227 | 3.802 |
| ATOM | 17 | C3 | MAN | 17 | 2.424 | 26.420 | 18.627 | −0.15 | +0.08 | +0.113 | 3.802 |
| ATOM | 18 | C2 | MAN | 17 | 3.614 | 27.327 | 18.330 | −0.33 | +0.09 | +0.113 | 3.802 |
| ATOM | 19 | O2 | MAN | 17 | 4.587 | 27.206 | 19.360 | +0.15 | −0.47 | −0.537 | 3.802 |
| ATOM | 20 | HO2 | MAN | 17 | 5.090 | 28.098 | 19.463 | +0.11 | +0.36 | +0.424 | 3.802 |
| ATOM | 21 | C1 | MAN | 17 | 3.160 | 28.765 | 18.228 | −0.29 | +0.18 | +0.227 | 3.802 |
| ATOM | 22 | O4 | NAG | 17 | 4.259 | 29.580 | 18.043 | +0.04 | −0.19 | −0.227 | 3.802 |
| ATOM | 23 | C4 | NAG | 17 | 4.266 | 30.847 | 18.714 | −0.23 | +0.09 | +0.113 | 3.802 |
| ATOM | 24 | C5 | NAG | 17 | 3.821 | 31.936 | 17.715 | −0.34 | +0.10 | +0.113 | 3.802 |
| ATOM | 25 | C6 | NAG | 17 | 2.325 | 31.956 | 17.446 | −0.52 | +0.13 | +0.113 | 3.802 |
| ATOM | 26 | O6 | NAG | 17 | 1.717 | 33.147 | 17.930 | −0.02 | −0.79 | −0.537 | 3.802 |
| ATOM | 27 | H6 | NAG | 17 | 2.405 | 33.913 | 17.920 | +0.08 | +0.40 | +0.424 | 3.802 |
| ATOM | 28 | O5 | NAG | 17 | 4.140 | 33.226 | 18.218 | +0.09 | −0.19 | −0.227 | 3.802 |
| ATOM | 29 | C1 | NAG | 17 | 5.530 | 33.399 | 18.402 | −0.18 | +0.09 | +0.113 | 3.802 |
| ATOM | 30 | C2 | NAG | 17 | 5.940 | 32.533 | 19.605 | −0.08 | +0.16 | +0.211 | 3.802 |
| ATOM | 31 | N2 | NAG | 17 | 7.341 | 32.698 | 19.924 | −0.02 | −0.48 | −0.650 | 3.802 |
| ATOM | 32 | HN2 | NAG | 17 | 7.980 | 33.110 | 19.231 | +0.12 | +0.34 | +0.440 | 3.802 |
| ATOM | 33 | C7 | NAG | 17 | 7.808 | 32.320 | 21.112 | −0.01 | +0.27 | +0.396 | 3.802 |
| ATOM | 34 | O7 | NAG | 17 | 7.097 | 31.821 | 21.983 | +0.23 | −0.26 | −0.396 | 3.802 |
| ATOM | 35 | C8 | NAG | 17 | 9.287 | 32.530 | 21.378 | +0.00 | +0.00 | +0.000 | 3.802 |
| ATOM | 36 | C3 | NAG | 17 | 5.679 | 31.073 | 19.254 | −0.12 | +0.09 | +0.113 | 3.802 |
| ATOM | 37 | O3 | NAG | 17 | 5.870 | 30.260 | 20.402 | +0.22 | −0.41 | −0.537 | 3.802 |
| ATOM | 38 | HO3 | NAG | 17 | 5.100 | 30.417 | 21.068 | +0.12 | +0.32 | +0.424 | 3.802 |
| ATOM | 39 | O5 | MAN | 17 | 2.478 | 29.145 | 19.433 | +0.06 | −0.17 | −0.227 | 3.802 |
| ATOM | 40 | C5 | MAN | 17 | 1.246 | 28.431 | 19.580 | −0.33 | +0.08 | +0.113 | 3.802 |
| ATOM | 41 | C6 | MAN | 17 | 0.394 | 29.156 | 20.630 | −0.30 | +0.09 | +0.113 | 3.802 |
| ATOM | 42 | O6 | MAN | 17 | 0.139 | 30.536 | 20.261 | +0.09 | −0.21 | −0.227 | 3.802 |
| ATOM | 43 | C1 | MAN | 17 | −0.093 | 31.415 | 21.343 | −0.12 | +0.20 | +0.227 | 3.802 |
| ATOM | 44 | O5 | MAN | 17 | 1.053 | 32.183 | 21.752 | +0.21 | −0.17 | −0.227 | 3.802 |
| ATOM | 45 | C5 | MAN | 17 | 1.433 | 33.264 | 20.862 | −0.11 | +0.09 | +0.113 | 3.802 |
| ATOM | 46 | C6 | MAN | 17 | 2.710 | 33.907 | 21.396 | −0.05 | +0.07 | +0.113 | 3.802 |
| ATOM | 47 | O6 | MAN | 17 | 2.941 | 33.514 | 22.763 | +0.23 | −0.14 | −0.227 | 3.802 |
| ATOM | 48 | C1 | MAN | 17 | 3.992 | 32.591 | 22.898 | +0.00 | +0.14 | +0.227 | 3.802 |
| ATOM | 49 | O5 | MAN | 17 | 5.228 | 33.213 | 23.261 | +0.24 | −0.14 | −0.227 | 3.802 |
| ATOM | 50 | C5 | MAN | 17 | 5.190 | 33.937 | 24.514 | +0.00 | +0.06 | +0.113 | 3.802 |
| ATOM | 51 | C6 | MAN | 17 | 6.572 | 34.516 | 24.740 | +0.00 | +0.06 | +0.113 | 3.802 |
| ATOM | 52 | O6 | MAN | 17 | 6.896 | 35.481 | 23.749 | +0.24 | −0.29 | −0.537 | 3.802 |
| ATOM | 53 | H6 | MAN | 17 | 6.519 | 35.179 | 22.839 | +0.12 | +0.24 | +0.424 | 3.802 |
| ATOM | 54 | C4 | MAN | 17 | 4.775 | 33.016 | 25.666 | −0.02 | +0.06 | +0.113 | 3.802 |
| ATOM | 55 | O4 | MAN | 17 | 4.535 | 33.813 | 26.814 | +0.23 | −0.27 | −0.537 | 3.802 |
| ATOM | 56 | HO4 | MAN | 17 | 3.712 | 33.451 | 27.317 | +0.12 | +0.21 | +0.424 | 3.802 |
| ATOM | 57 | C3 | MAN | 17 | 3.513 | 32.211 | 25.334 | −0.02 | +0.07 | +0.113 | 3.802 |
| ATOM | 58 | O3 | MAN | 17 | 3.309 | 31.215 | 26.325 | +0.21 | −0.31 | −0.537 | 3.802 |
| ATOM | 59 | HO3 | MAN | 17 | 2.335 | 31.256 | 26.659 | +0.12 | +0.25 | +0.424 | 3.802 |
| ATOM | 60 | C2 | MAN | 17 | 3.615 | 31.548 | 23.956 | −0.01 | +0.07 | +0.113 | 3.802 |
| ATOM | 61 | O2 | MAN | 17 | 4.588 | 30.514 | 23.986 | +0.23 | −0.36 | −0.537 | 3.802 |
| ATOM | 62 | HO2 | MAN | 17 | 4.655 | 30.134 | 24.942 | +0.12 | +0.27 | +0.424 | 3.802 |
| ATOM | 63 | C4 | MAN | 17 | 0.271 | 34.249 | 20.699 | −0.16 | +0.08 | +0.113 | 3.802 |
| ATOM | 64 | O4 | MAN | 17 | 0.650 | 35.309 | 19.833 | +0.11 | −0.17 | −0.537 | 3.802 |
| ATOM | 65 | HO4 | MAN | 17 | 0.951 | 36.120 | 20.392 | +0.08 | +0.14 | +0.424 | 3.802 |
| ATOM | 66 | C3 | MAN | 17 | −0.891 | 33.457 | 20.092 | −0.23 | +0.11 | +0.113 | 3.802 |
| ATOM | 67 | O3 | MAN | 17 | −2.020 | 34.300 | 19.731 | +0.07 | −0.15 | −0.227 | 3.802 |
| ATOM | 68 | C1 | BDG | 17 | −3.050 | 33.717 | 18.930 | −0.24 | +0.22 | +0.227 | 3.802 |
| ATOM | 69 | C2 | BDG | 17 | −3.660 | 34.791 | 18.000 | −0.27 | +0.08 | +0.113 | 3.802 |
| ATOM | 70 | O22 | BDG | 17 | −4.573 | 34.158 | 17.100 | +0.01 | −0.12 | −0.113 | 3.802 |
| ATOM | 71 | C3 | BDG | 17 | −4.430 | 35.859 | 18.813 | −0.24 | +0.06 | +0.113 | 3.802 |
| ATOM | 72 | O3 | BDG | 17 | −5.099 | 36.733 | 17.938 | +0.07 | −0.26 | −0.537 | 3.802 |
| ATOM | 73 | HO3 | BDG | 17 | −5.544 | 37.403 | 18.490 | +0.11 | +0.20 | +0.424 | 3.802 |
| ATOM | 74 | C4 | BDG | 17 | −5.447 | 35.230 | 19.801 | −0.08 | +0.08 | +0.113 | 3.802 |
| ATOM | 75 | O4 | BDG | 17 | −5.985 | 36.241 | 20.620 | +0.22 | −0.33 | −0.537 | 3.802 |
| ATOM | 76 | HO4 | BDG | 17 | −6.635 | 35.817 | 21.208 | +0.12 | +0.28 | +0.424 | 3.802 |
| ATOM | 77 | C5 | BDG | 17 | −4.772 | 34.118 | 20.659 | −0.09 | +0.09 | +0.113 | 3.802 |
| ATOM | 78 | C6 | BDG | 17 | −5.772 | 33.313 | 21.538 | −0.10 | +0.12 | +0.113 | 3.802 |
| ATOM | 79 | O6 | BDG | 17 | −6.347 | 34.126 | 22.538 | +0.21 | −0.46 | −0.537 | 3.802 |
| ATOM | 80 | H6 | BDG | 17 | −6.940 | 33.565 | 23.041 | +0.11 | +0.40 | +0.424 | 3.802 |
| ATOM | 81 | O | BDG | 17 | −4.082 | 33.174 | 19.802 | +0.16 | −0.22 | −0.227 | 3.802 |
| ATOM | 82 | C2 | MAN | 17 | −1.302 | 32.332 | 21.053 | −0.14 | +0.11 | +0.113 | 3.802 |
| ATOM | 83 | O2 | MAN | 17 | −1.769 | 32.894 | 22.272 | +0.20 | −0.46 | −0.537 | 3.802 |
| ATOM | 84 | HO2 | MAN | 17 | −2.269 | 32.174 | 22.813 | +0.11 | +0.42 | +0.424 | 3.802 |
| ATOM | 85 | C4 | MAN | 17 | 1.555 | 26.944 | 19.796 | −0.39 | +0.06 | +0.113 | 3.802 |
| ATOM | 86 | O4 | MAN | 17 | 0.340 | 26.209 | 19.850 | +0.03 | −0.19 | −0.537 | 3.802 |
| ATOM | 87 | HO4 | MAN | 17 | −0.457 | 26.856 | 19.769 | +0.06 | +0.26 | +0.424 | 3.802 |
| TER | | | | | | | | | | | |
| ENDMDL | | | | | | | | | | | |
| MODEL | 77 | | | | | | | | | | |
| USER | Run = 77 | | | | | | | | | | |
| USER | Cluster Rank = 18 | | | | | | | | | | |
| USER | Number of conformations in this cluster = 1 | | | | | | | | | | |
| USER | | | | | | | | | | | |
| USER | RMSD from reference structure = 3.879 A | | | | | | | | | | |
| USER | | | | | | | | | | | |
| USER | Estimated Free Energy of Binding = +2.14 kcal/mol [= (1) + (3)] | | | | | | | | | | |
| USER | Estimated Inhibition Constant, Ki = +37.33 [Temperature = 298.15 K] | | | | | | | | | | |
| USER | | | | | | | | | | | |
| USER | Final Docked Energy = −0.87 kcal/mol [= (1) + (2)] | | | | | | | | | | |
| USER | | | | | | | | | | | |
| USER | (1) Final Intermolecular Energy = −2.84 kcal/mol | | | | | | | | | | |
| USER | (2) Final Internal Energy of Ligand = +1.97 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 3.248817 27.654631 18.967217 | | | | | | | | | | |
| USER | NEWDPF quat0 0.671278 0.740263 −0.037362 −161.428510 | | | | | | | | | | |
| USER | NEWDPF ndihe 16 | | | | | | | | | | |
| USER | NEWDPF dihe0 −179.57 101.19 157.70 −64.86 −61.07 5.10 −37.65 −16.22 | | | | | | | | | | |
| | 97.65 15.98 −19.43 60.90 87.45 −54.23 56.63 −44.59 | | | | | | | | | | |
| USER | | | | | | | | | | | |
| USER | | | | Rank | x | y | z | vdW | Elec | q | RMS |
| ATOM | 1 | C5 | MAN | 18 | −1.705 | 30.455 | 22.980 | −0.10 | +0.14 | +0.113 | 3.879 |
| ATOM | 2 | HO4 | MAN | 18 | −1.137 | 31.729 | 20.695 | +0.11 | +0.43 | +0.424 | 3.879 |
| ATOM | 3 | O4 | MAN | 18 | −2.130 | 31.703 | 20.972 | +0.15 | −0.57 | −0.537 | 3.879 |
| ATOM | 4 | O5 | MAN | 18 | −1.738 | 29.129 | 23.556 | −0.21 | −0.33 | −0.227 | 3.879 |
| ATOM | 5 | HO2 | MAN | 18 | −3.392 | 26.641 | 21.860 | +0.02 | +0.38 | +0.424 | 3.879 |
| ATOM | 6 | O2 | MAN | 18 | −3.311 | 27.660 | 21.741 | −0.15 | −0.69 | −0.537 | 3.879 |
| ATOM | 7 | C2 | MAN | 18 | −1.969 | 27.995 | 21.445 | −0.55 | +0.11 | +0.113 | 3.879 |
| ATOM | 8 | C1 | MAN | 18 | −1.170 | 28.109 | 22.731 | −0.40 | +0.23 | +0.227 | 3.879 |
| ATOM | 9 | C4 | MAN | 18 | −2.409 | 30.471 | 21.613 | −0.01 | +0.15 | +0.113 | 3.879 |
| ATOM | 10 | O3 | MAN | 18 | −2.730 | 29.256 | 19.549 | −0.04 | −0.56 | −0.537 | 3.879 |
| ATOM | 11 | HO3 | MAN | 18 | −2.335 | 28.563 | 18.897 | +0.09 | +0.43 | +0.424 | 3.879 |
| ATOM | 12 | C3 | MAN | 18 | −1.922 | 29.328 | 20.711 | −0.39 | +0.12 | +0.113 | 3.879 |
| ATOM | 13 | C6 | MAN | 18 | −2.368 | 31.391 | 23.977 | −0.10 | +0.12 | +0.113 | 3.879 |
| ATOM | 14 | O6 | MAN | 18 | −1.912 | 32.729 | 23.815 | +0.21 | −0.44 | −0.537 | 3.879 |
| ATOM | 15 | H6 | MAN | 18 | −1.198 | 32.937 | 24.527 | +0.12 | +0.31 | +0.424 | 3.879 |
| ATOM | 16 | O3 | MAN | 18 | 0.152 | 28.387 | 22.397 | +0.10 | −0.18 | −0.227 | 3.879 |
| ATOM | 17 | C3 | MAN | 18 | 1.137 | 27.786 | 23.241 | −0.28 | +0.09 | +0.113 | 3.879 |
| ATOM | 18 | C2 | MAN | 18 | 1.203 | 28.600 | 24.528 | −0.16 | +0.09 | +0.113 | 3.879 |
| ATOM | 19 | O2 | MAN | 18 | 1.309 | 29.984 | 24.224 | +0.21 | −0.40 | −0.537 | 3.879 |
| ATOM | 20 | HO2 | MAN | 18 | 1.842 | 30.459 | 24.967 | +0.12 | +0.30 | +0.424 | 3.879 |
| ATOM | 21 | C1 | MAN | 18 | 2.392 | 28.170 | 25.356 | −0.20 | +0.19 | +0.227 | 3.879 |
| ATOM | 22 | O4 | NAG | 18 | 2.485 | 28.972 | 26.477 | +0.17 | −0.16 | −0.227 | 3.879 |
| ATOM | 23 | C4 | NAG | 18 | 3.557 | 29.921 | 26.526 | −0.13 | +0.07 | +0.113 | 3.879 |
| ATOM | 24 | C5 | NAG | 18 | 4.710 | 29.320 | 27.358 | −0.24 | +0.05 | +0.113 | 3.879 |
| ATOM | 25 | C6 | NAG | 18 | 5.522 | 28.267 | 26.622 | −0.24 | +0.06 | +0.113 | 3.879 |
| ATOM | 26 | O6 | NAG | 18 | 6.857 | 28.697 | 26.386 | +0.16 | −0.25 | −0.537 | 3.879 |
| ATOM | 27 | H6 | NAG | 18 | 7.138 | 29.361 | 27.123 | +0.10 | +0.15 | +0.424 | 3.879 |
| ATOM | 28 | O5 | NAG | 18 | 5.635 | 30.335 | 27.723 | +0.10 | −0.09 | −0.227 | 3.879 |
| ATOM | 29 | C1 | NAG | 18 | 5.042 | 31.334 | 28.528 | +0.02 | +0.05 | +0.113 | 3.879 |
| ATOM | 30 | C2 | NAG | 18 | 4.099 | 32.144 | 27.624 | −0.12 | +0.10 | +0.211 | 3.879 |
| ATOM | 31 | N2 | NAG | 18 | 3.494 | 33.246 | 28.340 | −0.06 | −0.31 | −0.650 | 3.879 |
| ATOM | 32 | HN2 | NAG | 18 | 2.775 | 33.061 | 29.053 | +0.11 | +0.20 | +0.440 | 3.879 |
| ATOM | 33 | C7 | NAG | 18 | 3.863 | 34.498 | 28.082 | −0.04 | +0.19 | +0.396 | 3.879 |
| ATOM | 34 | O7 | NAG | 18 | 4.715 | 34.785 | 27.242 | +0.23 | −0.19 | −0.396 | 3.879 |
| ATOM | 35 | C8 | NAG | 18 | 3.190 | 35.602 | 28.877 | −0.02 | +0.00 | +0.000 | 3.879 |
| ATOM | 36 | C3 | NAG | 18 | 3.000 | 31.214 | 27.124 | −0.11 | +0.06 | +0.113 | 3.879 |
| ATOM | 37 | O3 | NAG | 18 | 2.217 | 31.877 | 26.142 | +0.22 | −0.32 | −0.537 | 3.879 |
| ATOM | 38 | HO3 | NAG | 18 | 2.765 | 31.978 | 25.275 | +0.12 | +0.26 | +0.424 | 3.879 |
| ATOM | 39 | O5 | MAN | 18 | 3.593 | 28.290 | 24.579 | +0.17 | −0.20 | −0.227 | 3.879 |
| ATOM | 40 | C5 | MAN | 18 | 3.606 | 27.357 | 23.494 | −0.16 | +0.14 | +0.113 | 3.879 |
| ATOM | 41 | C6 | MAN | 18 | 5.044 | 27.250 | 22.969 | −0.14 | +0.14 | +0.113 | 3.879 |
| ATOM | 42 | O6 | MAN | 18 | 5.261 | 28.099 | 21.812 | +0.20 | −0.20 | −0.227 | 3.879 |
| ATOM | 43 | C1 | MAN | 18 | 5.923 | 27.480 | 20.727 | −0.08 | +0.21 | +0.227 | 3.879 |
| ATOM | 44 | O5 | MAN | 18 | 5.103 | 26.590 | 19.950 | +0.17 | −0.22 | −0.227 | 3.879 |
| ATOM | 45 | C5 | MAN | 18 | 4.130 | 27.229 | 19.084 | −0.24 | +0.10 | +0.113 | 3.879 |
| ATOM | 46 | C6 | MAN | 18 | 3.292 | 26.146 | 18.410 | −0.38 | +0.09 | +0.113 | 3.879 |
| ATOM | 47 | O6 | MAN | 18 | 3.966 | 24.873 | 18.471 | +0.08 | −0.21 | −0.227 | 3.879 |
| ATOM | 48 | C1 | MAN | 18 | 4.152 | 24.289 | 17.208 | −0.39 | +0.26 | +0.227 | 3.879 |
| ATOM | 49 | O5 | MAN | 18 | 5.051 | 25.035 | 16.383 | +0.00 | −0.42 | −0.227 | 3.879 |
| ATOM | 50 | C5 | MAN | 18 | 6.381 | 25.191 | 16.933 | −0.31 | +0.15 | +0.113 | 3.879 |
| ATOM | 51 | C6 | MAN | 18 | 7.191 | 25.990 | 15.933 | −0.39 | +0.15 | +0.113 | 3.879 |
| ATOM | 52 | O6 | MAN | 18 | 6.437 | 27.071 | 15.404 | −0.03 | −0.69 | −0.537 | 3.879 |
| ATOM | 53 | H6 | MAN | 18 | 5.457 | 26.778 | 15.278 | −0.17 | +0.53 | +0.424 | 3.879 |
| ATOM | 54 | C4 | MAN | 18 | 7.022 | 23.826 | 17.208 | −0.33 | +0.13 | +0.113 | 3.879 |
| ATOM | 55 | O4 | MAN | 18 | 8.215 | 24.031 | 17.948 | +0.11 | −0.55 | −0.537 | 3.879 |
| ATOM | 56 | HO4 | MAN | 18 | 8.304 | 23.298 | 18.665 | +0.09 | +0.42 | +0.424 | 3.879 |
| ATOM | 57 | C3 | MAN | 18 | 6.089 | 22.900 | 17.996 | −0.28 | +0.11 | +0.113 | 3.879 |
| ATOM | 58 | O3 | MAN | 18 | 6.630 | 21.587 | 18.019 | +0.04 | −0.53 | −0.537 | 3.879 |
| ATOM | 59 | HO3 | MAN | 18 | 6.636 | 21.234 | 18.988 | +0.07 | +0.42 | +0.424 | 3.879 |
| ATOM | 60 | C2 | MAN | 18 | 4.685 | 22.863 | 17.384 | −0.32 | +0.11 | +0.113 | 3.879 |
| ATOM | 61 | O2 | MAN | 18 | 4.719 | 22.199 | 16.130 | +0.12 | −0.51 | −0.537 | 3.879 |
| ATOM | 62 | HO2 | MAN | 18 | 5.493 | 21.518 | 16.123 | −0.06 | +0.33 | +0.424 | 3.879 |
| ATOM | 63 | C4 | MAN | 18 | 4.834 | 28.165 | 18.096 | −0.28 | +0.10 | +0.113 | 3.879 |
| ATOM | 64 | O4 | MAN | 18 | 3.879 | 28.783 | 17.246 | −0.04 | −0.47 | −0.537 | 3.879 |
| ATOM | 65 | HO4 | MAN | 18 | 3.823 | 28.266 | 16.357 | +0.04 | +0.43 | +0.424 | 3.879 |
| ATOM | 66 | C3 | MAN | 18 | 5.560 | 29.220 | 18.935 | −0.15 | +0.10 | +0.113 | 3.879 |
| ATOM | 67 | O3 | MAN | 18 | 6.148 | 30.276 | 18.126 | +0.13 | −0.21 | −0.227 | 3.879 |
| ATOM | 68 | C1 | BDG | 18 | 5.499 | 31.549 | 18.128 | −0.19 | +0.20 | +0.227 | 3.879 |
| ATOM | 69 | C2 | BDG | 18 | 6.543 | 32.670 | 17.921 | −0.15 | +0.11 | +0.113 | 3.879 |
| ATOM | 70 | O22 | BDG | 18 | 5.860 | 33.916 | 17.767 | +0.12 | −0.10 | −0.113 | 3.879 |
| ATOM | 71 | C3 | BDG | 18 | 7.495 | 32.776 | 19.136 | −0.07 | +0.09 | +0.113 | 3.879 |
| ATOM | 72 | O3 | BDG | 18 | 8.333 | 33.895 | 18.989 | +0.22 | −0.42 | −0.537 | 3.879 |
| ATOM | 73 | HO3 | BDG | 18 | 8.933 | 33.905 | 19.758 | +0.12 | +0.30 | +0.424 | 3.879 |
| ATOM | 74 | C4 | BDG | 18 | 6.729 | 32.871 | 20.480 | −0.03 | +0.08 | +0.113 | 3.879 |
| ATOM | 75 | O4 | BDG | 18 | 7.647 | 32.795 | 21.545 | +0.23 | −0.35 | −0.537 | 3.879 |
| ATOM | 76 | HO4 | BDG | 18 | 7.137 | 32.883 | 22.371 | +0.12 | +0.26 | +0.424 | 3.879 |
| ATOM | 77 | C5 | BDG | 18 | 5.656 | 31.747 | 20.588 | −0.04 | +0.08 | +0.113 | 3.879 |
| ATOM | 78 | C6 | BDG | 18 | 4.720 | 31.892 | 21.823 | −0.02 | +0.08 | +0.113 | 3.879 |
| ATOM | 79 | O6 | BDG | 18 | 5.430 | 31.716 | 23.030 | +0.24 | −0.34 | −0.537 | 3.879 |
| ATOM | 80 | H6 | BDG | 18 | 4.793 | 31.810 | 23.740 | +0.12 | +0.26 | +0.424 | 3.879 |
| ATOM | 81 | O | BDG | 18 | 4.820 | 31.736 | 19.403 | +0.18 | −0.18 | −0.227 | 3.879 |
| ATOM | 82 | C2 | MAN | 18 | 6.605 | 28.535 | 19.828 | −0.06 | +0.09 | +0.113 | 3.879 |
| ATOM | 83 | O2 | MAN | 18 | 7.583 | 27.900 | 19.017 | +0.21 | −0.47 | −0.537 | 3.879 |
| ATOM | 84 | HO2 | MAN | 18 | 8.403 | 27.658 | 19.591 | +0.12 | +0.35 | +0.424 | 3.879 |
| ATOM | 85 | C4 | MAN | 18 | 2.497 | 27.732 | 22.501 | −0.23 | +0.10 | +0.113 | 3.879 |
| ATOM | 86 | O4 | MAN | 18 | 2.436 | 26.761 | 21.467 | +0.29 | −0.33 | −0.537 | 3.879 |
| ATOM | 87 | HO4 | MAN | 18 | 3.176 | 26.058 | 21.611 | −0.17 | +0.53 | +0.424 | 3.879 |
| TER | | | | | | | | | | | |
| ENDMDL | | | | | | | | | | | |
| MODEL | 29 | | | | | | | | | | |
| USER | Run = 29 | | | | | | | | | | |
| USER | Cluster Rank = 19 | | | | | | | | | | |
| USER | Number of conformations in this cluster = 1 | | | | | | | | | | |
| USER | | | | | | | | | | | |
| USER | RMSD from reference structure = 4.102 A | | | | | | | | | | |
| USER | | | | | | | | | | | |
| USER | Estimated Free Energy of Binding = −0.98 kcal/mol [= (1) + (3)] | | | | | | | | | | |
| USER | Estimated Inhibition Constant, Ki = +0.19 [Temperature = 298.15 K] | | | | | | | | | | |
| USER | | | | | | | | | | | |
| USER | Final Docked Energy = −0.82 kcal/mol [= (1) + (2)] | | | | | | | | | | |
| USER | | | | | | | | | | | |
| USER | (1) Final Intermolecular Energy = −5.96 kcal/mol | | | | | | | | | | |
| USER | (2) Final Internal Energy of Ligand = +5.14 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 3.377977 28.795214 21.588594 | | | | | | | | | | |
| USER | NEWDPF quat0 −0.744163 −0.500500 0.442404 91.259198 | | | | | | | | | | |
| USER | NEWDPF ndihe 16 | | | | | | | | | | |
| USER | NEWDPF dihe0 163.62 120.79 −120.43 −18.35 −11.24 −35.17 12.90 −38.64 | | | | | | | | | | |
| | 102.40 −55.53 −179.95 −179.37 35.84 −175.02 19.39 16.24 | | | | | | | | | | |
| USER | | | | | | | | | | | |
| USER | | | | Rank | x | y | z | vdW | Elec | q | RMS |
| ATOM | 1 | C5 | MAN | 19 | 7.425 | 29.674 | 27.187 | −0.17 | +0.04 | +0.113 | 4.102 |
| ATOM | 2 | HO4 | MAN | 19 | 5.926 | 31.762 | 26.439 | +0.12 | +0.22 | +0.424 | 4.102 |
| ATOM | 3 | O4 | MAN | 19 | 6.028 | 31.609 | 27.453 | +0.17 | −0.26 | −0.537 | 4.102 |
| ATOM | 4 | O5 | MAN | 19 | 7.424 | 28.227 | 27.193 | +0.11 | −0.06 | −0.227 | 4.102 |
| ATOM | 5 | HO2 | MAN | 19 | 4.674 | 26.590 | 28.440 | −0.20 | +0.01 | +0.424 | 4.102 |
| ATOM | 6 | O2 | MAN | 19 | 4.977 | 27.573 | 28.420 | −0.07 | −0.07 | −0.537 | 4.102 |
| ATOM | 7 | C2 | MAN | 19 | 5.028 | 28.024 | 27.081 | −0.32 | +0.05 | +0.113 | 4.102 |
| ATOM | 8 | C1 | MAN | 19 | 6.355 | 27.631 | 26.455 | −0.25 | +0.12 | +0.227 | 4.102 |
| ATOM | 9 | C4 | MAN | 19 | 6.089 | 30.219 | 27.718 | −0.20 | +0.04 | +0.113 | 4.102 |
| ATOM | 10 | O3 | MAN | 19 | 3.691 | 29.920 | 27.695 | +0.12 | −0.26 | −0.537 | 4.102 |
| ATOM | 11 | HO3 | MAN | 19 | 2.882 | 29.552 | 27.173 | +0.11 | +0.25 | +0.424 | 4.102 |
| ATOM | 12 | C3 | MAN | 19 | 4.889 | 29.539 | 27.042 | −0.20 | +0.05 | +0.113 | 4.102 |
| ATOM | 13 | C6 | MAN | 19 | 8.604 | 30.123 | 28.033 | −0.16 | +0.04 | +0.113 | 4.102 |
| ATOM | 14 | O6 | MAN | 19 | 8.764 | 31.535 | 27.993 | +0.19 | −0.25 | −0.537 | 4.102 |
| ATOM | 15 | H6 | MAN | 19 | 9.671 | 31.767 | 27.564 | +0.12 | +0.20 | +0.424 | 4.102 |
| ATOM | 16 | O3 | MAN | 19 | 6.346 | 28.044 | 25.126 | +0.19 | −0.16 | −0.227 | 4.102 |
| ATOM | 17 | C3 | MAN | 19 | 5.709 | 27.149 | 24.210 | −0.17 | +0.12 | +0.113 | 4.102 |
| ATOM | 18 | C2 | MAN | 19 | 6.777 | 26.625 | 23.256 | −0.13 | +0.11 | +0.113 | 4.102 |
| ATOM | 19 | O2 | MAN | 19 | 7.544 | 27.706 | 22.742 | +0.22 | −0.42 | −0.537 | 4.102 |
| ATOM | 20 | HO2 | MAN | 19 | 7.873 | 27.473 | 21.794 | +0.12 | +0.34 | +0.424 | 4.102 |
| ATOM | 21 | C1 | MAN | 19 | 6.132 | 25.873 | 22.115 | −0.14 | +0.30 | +0.227 | 4.102 |
| ATOM | 22 | O4 | NAG | 19 | 7.105 | 25.477 | 21.218 | +0.18 | −0.23 | −0.227 | 4.102 |
| ATOM | 23 | C4 | NAG | 19 | 6.684 | 24.745 | 20.061 | −0.15 | +0.12 | +0.113 | 4.102 |
| ATOM | 24 | C5 | NAG | 19 | 7.380 | 23.368 | 20.069 | −0.27 | +0.11 | +0.113 | 4.102 |
| ATOM | 25 | C6 | NAG | 19 | 6.784 | 22.377 | 21.056 | −0.43 | +0.12 | +0.113 | 4.102 |
| ATOM | 26 | O6 | NAG | 19 | 6.172 | 21.277 | 20.396 | −0.03 | −0.61 | −0.537 | 4.102 |
| ATOM | 27 | H6 | NAG | 19 | 6.637 | 21.117 | 19.491 | +0.07 | +0.42 | +0.424 | 4.102 |
| ATOM | 28 | O5 | NAG | 19 | 7.271 | 22.753 | 18.792 | +0.07 | −0.22 | −0.227 | 4.102 |
| ATOM | 29 | C1 | NAG | 19 | 7.899 | 23.512 | 17.779 | −0.32 | +0.12 | +0.113 | 4.102 |
| ATOM | 30 | C2 | NAG | 19 | 7.037 | 24.764 | 17.548 | −0.25 | +0.23 | +0.211 | 4.102 |
| ATOM | 31 | N2 | NAG | 19 | 7.558 | 25.576 | 16.471 | −0.20 | −0.81 | −0.650 | 4.102 |
| ATOM | 32 | HN2 | NAG | 19 | 8.557 | 25.525 | 16.227 | +0.10 | +0.54 | +0.440 | 4.102 |
| ATOM | 33 | C7 | NAG | 19 | 6.752 | 26.388 | 15.791 | −0.36 | +0.53 | +0.396 | 4.102 |
| ATOM | 34 | O7 | NAG | 19 | 5.550 | 26.494 | 16.033 | +0.00 | −0.53 | −0.396 | 4.102 |
| ATOM | 35 | C8 | NAG | 19 | 7.368 | 27.204 | 14.669 | −0.52 | +0.00 | +0.000 | 4.102 |
| ATOM | 36 | C3 | NAG | 19 | 7.034 | 25.585 | 18.832 | −0.12 | +0.11 | +0.113 | 4.102 |
| ATOM | 37 | O3 | NAG | 19 | 6.103 | 26.651 | 18.720 | +0.18 | −0.51 | −0.537 | 4.102 |
| ATOM | 38 | HO3 | NAG | 19 | 5.144 | 26.276 | 18.753 | +0.11 | +0.41 | +0.424 | 4.102 |
| ATOM | 39 | O5 | MAN | 19 | 5.184 | 26.720 | 21.450 | +0.16 | −0.26 | −0.227 | 4.102 |
| ATOM | 40 | C5 | MAN | 19 | 4.073 | 27.024 | 22.300 | −0.05 | +0.15 | +0.113 | 4.102 |
| ATOM | 41 | C6 | MAN | 19 | 2.934 | 27.568 | 21.427 | −0.23 | +0.09 | +0.113 | 4.102 |
| ATOM | 42 | O6 | MAN | 19 | 1.636 | 27.106 | 21.882 | +0.26 | −0.11 | −0.227 | 4.102 |
| ATOM | 43 | C1 | MAN | 19 | 0.528 | 27.647 | 21.191 | −0.37 | +0.13 | +0.227 | 4.102 |
| ATOM | 44 | O5 | MAN | 19 | −0.737 | 27.481 | 21.855 | −0.01 | −0.16 | −0.227 | 4.102 |
| ATOM | 45 | C5 | MAN | 19 | −0.953 | 28.330 | 23.012 | −0.34 | +0.11 | +0.113 | 4.102 |
| ATOM | 46 | C6 | MAN | 19 | −2.292 | 27.960 | 23.643 | −0.10 | +0.19 | +0.113 | 4.102 |
| ATOM | 47 | O6 | MAN | 19 | −2.558 | 28.791 | 24.790 | −0.16 | −0.38 | −0.227 | 4.102 |
| ATOM | 48 | C1 | MAN | 19 | −2.689 | 28.057 | 25.981 | −0.27 | +0.32 | +0.227 | 4.102 |
| ATOM | 49 | O5 | MAN | 19 | −1.981 | 26.815 | 25.943 | −0.14 | −0.25 | −0.227 | 4.102 |
| ATOM | 50 | C5 | MAN | 19 | −0.556 | 26.944 | 25.727 | −0.50 | +0.11 | +0.113 | 4.102 |
| ATOM | 51 | C6 | MAN | 19 | 0.027 | 25.545 | 25.725 | −0.65 | +0.11 | +0.113 | 4.102 |
| ATOM | 52 | O6 | MAN | 19 | 0.750 | 25.288 | 24.530 | +0.14 | −0.51 | −0.537 | 4.102 |
| ATOM | 53 | H6 | MAN | 19 | 0.269 | 25.733 | 23.735 | +0.05 | +0.24 | +0.424 | 4.102 |
| ATOM | 54 | C4 | MAN | 19 | 0.086 | 27.815 | 26.812 | −0.36 | +0.12 | +0.113 | 4.102 |
| ATOM | 55 | O4 | MAN | 19 | 1.423 | 28.094 | 26.433 | +0.12 | −0.46 | −0.537 | 4.102 |
| ATOM | 56 | HO4 | MAN | 19 | 1.652 | 29.069 | 26.671 | +0.11 | +0.31 | +0.424 | 4.102 |
| ATOM | 57 | C3 | MAN | 19 | −0.670 | 29.133 | 27.014 | −0.22 | +0.10 | +0.113 | 4.102 |
| ATOM | 58 | O3 | MAN | 19 | −0.182 | 29.788 | 28.176 | +0.15 | −0.40 | −0.537 | 4.102 |
| ATOM | 59 | HO3 | MAN | 19 | 0.036 | 30.770 | 27.952 | +0.11 | +0.27 | +0.424 | 4.102 |
| ATOM | 60 | C2 | MAN | 19 | −2.178 | 28.901 | 27.153 | −0.33 | +0.12 | +0.113 | 4.102 |
| ATOM | 61 | O2 | MAN | 19 | −2.457 | 28.246 | 28.382 | −0.21 | −0.83 | −0.537 | 4.102 |
| ATOM | 62 | HO2 | MAN | 19 | −1.729 | 28.485 | 29.071 | +0.07 | +0.52 | +0.424 | 4.102 |
| ATOM | 63 | C4 | MAN | 19 | −0.848 | 29.805 | 22.613 | −0.19 | +0.11 | +0.113 | 4.102 |
| ATOM | 64 | O4 | MAN | 19 | −1.068 | 30.633 | 23.746 | +0.16 | −0.53 | −0.537 | 4.102 |
| ATOM | 65 | HO4 | MAN | 19 | −2.049 | 30.947 | 23.759 | +0.10 | +0.50 | +0.424 | 4.102 |
| ATOM | 66 | C3 | MAN | 19 | 0.568 | 30.012 | 22.071 | −0.12 | +0.09 | +0.113 | 4.102 |
| ATOM | 67 | O3 | MAN | 19 | 0.869 | 31.409 | 21.796 | +0.21 | −0.18 | −0.227 | 4.102 |
| ATOM | 68 | C1 | BDG | 19 | 2.243 | 31.801 | 21.796 | −0.05 | +0.17 | +0.227 | 4.102 |
| ATOM | 69 | C2 | BDG | 19 | 2.349 | 33.334 | 21.972 | −0.03 | +0.07 | +0.113 | 4.102 |
| ATOM | 70 | O22 | BDG | 19 | 3.726 | 33.687 | 22.128 | +0.23 | −0.07 | −0.113 | 4.102 |
| ATOM | 71 | C3 | BDG | 19 | 1.788 | 34.076 | 20.736 | −0.12 | +0.08 | +0.113 | 4.102 |
| ATOM | 72 | O3 | BDG | 19 | 2.034 | 35.455 | 20.855 | +0.19 | −0.28 | −0.537 | 4.102 |
| ATOM | 73 | HO3 | BDG | 19 | 1.639 | 35.884 | 20.073 | +0.10 | +0.17 | +0.424 | 4.102 |
| ATOM | 74 | C4 | BDG | 19 | 2.390 | 33.551 | 19.407 | −0.23 | +0.09 | +0.113 | 4.102 |
| ATOM | 75 | O4 | BDG | 19 | 1.717 | 34.147 | 18.324 | −0.07 | −0.53 | −0.537 | 4.102 |
| ATOM | 76 | HO4 | BDG | 19 | 2.136 | 33.816 | 17.509 | +0.06 | +0.47 | +0.424 | 4.102 |
| ATOM | 77 | C5 | BDG | 19 | 2.299 | 31.997 | 19.332 | −0.24 | +0.10 | +0.113 | 4.102 |
| ATOM | 78 | C6 | BDG | 19 | 3.053 | 31.385 | 18.116 | −0.40 | +0.10 | +0.113 | 4.102 |
| ATOM | 79 | O6 | BDG | 19 | 2.450 | 31.758 | 16.896 | −0.16 | −0.60 | −0.537 | 4.102 |
| ATOM | 80 | H6 | BDG | 19 | 2.958 | 31.340 | 16.199 | +0.02 | +0.40 | +0.424 | 4.102 |
| ATOM | 81 | O | BDG | 19 | 2.852 | 31.405 | 20.535 | +0.20 | −0.18 | −0.227 | 4.102 |
| ATOM | 82 | C2 | MAN | 19 | 0.778 | 29.129 | 20.832 | −0.26 | +0.09 | +0.113 | 4.102 |
| ATOM | 83 | O2 | MAN | 19 | −0.124 | 29.523 | 19.808 | +0.02 | −0.45 | −0.537 | 4.102 |
| ATOM | 84 | HO2 | MAN | 19 | 0.173 | 29.113 | 18.911 | +0.07 | +0.29 | +0.424 | 4.102 |
| ATOM | 85 | C4 | MAN | 19 | 4.563 | 27.884 | 23.473 | −0.14 | +0.11 | +0.113 | 4.102 |
| ATOM | 86 | O4 | MAN | 19 | 3.490 | 28.115 | 24.375 | +0.15 | −0.49 | −0.537 | 4.102 |
| ATOM | 87 | HO4 | MAN | 19 | 2.632 | 27.681 | 24.006 | +0.10 | +0.42 | +0.424 | 4.102 |
| TER | | | | | | | | | | | |
| ENDMDL | | | | | | | | | | | |
| MODEL | 38 | | | | | | | | | | |
| USER | Run = 38 | | | | | | | | | | |
| USER | Cluster Rank = 20 | | | | | | | | | | |
| USER | Number of conformations in this cluster = 1 | | | | | | | | | | |
| USER | | | | | | | | | | | |
| USER | RMSD from reference structure = 3.524 A | | | | | | | | | | |
| USER | | | | | | | | | | | |
| USER | Estimated Free Energy of Binding = −2.13 kcal/mol [= (1)+(3)] | | | | | | | | | | |
| USER | Estimated Inhibition Constant, Ki = +0.03 [Temperature = 298.15 K] | | | | | | | | | | |
| USER | | | | | | | | | | | |
| USER | Final Docked Energy = −0.67 kcal/mol [= (1)+(2)] | | | | | | | | | | |
| USER | | | | | | | | | | | |
| USER | (1) Final Intermolecular Energy = −7.11 kcal/mol | | | | | | | | | | |
| USER | (2) Final Internal Energy of Ligand = +6.44 kcal/mol | | | | | | | | | | |
| USER | (3) Torsional Free Energy = +4.98 kcal/mol | | | | | | | | | | |
| USER | | | | | | | | | | | |
| USER | | | | | | | | | | | |
| USER | DPF = new_oli.dpf | | | | | | | | | | |
| USER | NEWDPF move mohan_oligo.pdbq | | | | | | | | | | |
| USER | NEWDPF about 0.538000 27.870001 21.982000 | | | | | | | | | | |
| USER | NEWDPF tran0 1.809902 28.713868 20.809544 | | | | | | | | | | |
| USER | NEWDPF quat0 0.412073 −0.865080 −0.286064 90.572917 | | | | | | | | | | |
| USER | NEWDPF ndihe 16 | | | | | | | | | | |
| USER | NEWDPF dihe0 3.59 57.56 0.75 −73.86 −20.11 74.70 141.64 −33.42 100.34 | | | | | | | | | | |
| | −132.59 151.73 −29.72 33.66 −109.07 18.36 30.97 | | | | | | | | | | |
| USER | | | | | | | | | | | |
| USER | | | | Rank | x | y | z | vdW | Elec | q | RMS |
| ATOM | 1 | C5 | MAN | 20 | 4.740 | 22.461 | 19.910 | −0.35 | +0.13 | +0.113 | 3.524 |
| ATOM | 2 | HO4 | MAN | 20 | 2.602 | 22.574 | 21.518 | −0.26 | +0.30 | +0.424 | 3.524 |
| ATOM | 3 | O4 | MAN | 20 | 2.678 | 21.744 | 20.911 | −0.13 | −0.49 | −0.537 | 3.524 |
| ATOM | 4 | O5 | MAN | 20 | 5.308 | 23.094 | 18.740 | +0.13 | −0.23 | −0.227 | 3.524 |
| ATOM | 5 | HO2 | MAN | 20 | 3.338 | 23.466 | 15.951 | +0.08 | +0.56 | +0.424 | 3.524 |
| ATOM | 6 | O2 | MAN | 20 | 3.230 | 22.970 | 16.846 | +0.01 | −0.48 | −0.537 | 3.524 |
| ATOM | 7 | C2 | MAN | 20 | 3.202 | 23.908 | 17.904 | −0.42 | +0.09 | +0.113 | 3.524 |
| ATOM | 8 | C1 | MAN | 20 | 4.620 | 24.270 | 18.308 | −0.28 | +0.23 | +0.227 | 3.524 |
| ATOM | 9 | C4 | MAN | 20 | 3.260 | 22.115 | 19.674 | −0.46 | +0.09 | +0.113 | 3.524 |
| ATOM | 10 | O3 | MAN | 20 | 1.183 | 22.894 | 18.715 | −0.04 | +0.03 | −0.537 | 3.524 |
| ATOM | 11 | HO3 | MAN | 20 | 0.628 | 23.715 | 18.437 | −0.06 | +0.09 | +0.424 | 3.524 |
| ATOM | 12 | C3 | MAN | 20 | 2.482 | 23.309 | 19.104 | −0.48 | +0.06 | +0.113 | 3.524 |
| ATOM | 13 | C6 | MAN | 20 | 5.578 | 21.228 | 20.202 | −0.42 | +0.13 | +0.113 | 3.524 |
| ATOM | 14 | O6 | MAN | 20 | 6.319 | 21.377 | 21.407 | −0.12 | −0.71 | −0.537 | 3.524 |
| ATOM | 15 | H6 | MAN | 20 | 5.721 | 21.140 | 22.210 | +0.08 | +0.83 | +0.424 | 3.524 |
| ATOM | 16 | O3 | MAN | 20 | 4.552 | 25.232 | 19.313 | +0.12 | −0.22 | −0.227 | 3.524 |
| ATOM | 17 | C3 | MAN | 20 | 4.069 | 26.514 | 18.903 | −0.28 | +0.10 | +0.113 | 3.524 |
| ATOM | 18 | C2 | MAN | 20 | 5.204 | 27.516 | 19.087 | −0.15 | +0.10 | +0.113 | 3.524 |
| ATOM | 19 | O2 | MAN | 20 | 5.771 | 27.376 | 20.383 | +0.20 | −0.49 | −0.537 | 3.524 |
| ATOM | 20 | HO2 | MAN | 20 | 6.128 | 28.288 | 20.702 | +0.12 | +0.36 | +0.424 | 3.524 |
| ATOM | 21 | C1 | MAN | 20 | 4.687 | 28.925 | 18.909 | −0.19 | +0.19 | +0.227 | 3.524 |
| ATOM | 22 | O4 | NAG | 20 | 5.706 | 29.823 | 19.162 | +0.18 | −0.19 | −0.227 | 3.524 |
| ATOM | 23 | C4 | NAG | 20 | 6.343 | 30.438 | 18.035 | −0.17 | +0.11 | +0.113 | 3.524 |
| ATOM | 24 | C5 | NAG | 20 | 7.814 | 29.974 | 17.987 | −0.13 | +0.11 | +0.113 | 3.524 |
| ATOM | 25 | C6 | NAG | 20 | 8.001 | 28.558 | 17.466 | −0.15 | +0.12 | +0.113 | 3.524 |
| ATOM | 26 | O6 | NAG | 20 | 8.691 | 28.538 | 16.223 | +0.12 | −0.66 | −0.537 | 3.524 |
| ATOM | 27 | H6 | NAG | 20 | 9.313 | 29.357 | 16.163 | +0.11 | +0.53 | +0.424 | 3.524 |
| ATOM | 28 | O5 | NAG | 20 | 8.566 | 30.807 | 17.115 | +0.15 | −0.26 | −0.227 | 3.524 |
| ATOM | 29 | C1 | NAG | 20 | 8.586 | 32.152 | 17.548 | −0.14 | +0.12 | +0.113 | 3.524 |
| ATOM | 30 | C2 | NAG | 20 | 7.178 | 32.725 | 17.319 | −0.05 | +0.25 | +0.211 | 3.524 |
| ATOM | 31 | N2 | NAG | 20 | 7.113 | 34.128 | 17.664 | −0.10 | −0.62 | −0.650 | 3.524 |
| ATOM | 32 | HN2 | NAG | 20 | 7.011 | 34.409 | 18.649 | +0.11 | +0.35 | +0.440 | 3.524 |
| ATOM | 33 | C7 | NAG | 20 | 7.183 | 35.058 | 16.714 | −0.22 | +0.41 | +0.396 | 3.524 |
| ATOM | 34 | O7 | NAG | 20 | 7.304 | 34.786 | 15.520 | +0.05 | +0.60 | −0.396 | 3.524 |
| ATOM | 35 | C8 | NAG | 20 | 7.115 | 36.509 | 17.153 | −0.11 | +0.00 | +0.000 | 3.524 |
| ATOM | 36 | C3 | NAG | 20 | 6.199 | 31.952 | 18.195 | −0.16 | +0.10 | +0.113 | 3.524 |
| ATOM | 37 | O3 | NAG | 20 | 4.867 | 32.326 | 17.875 | +0.09 | −0.47 | −0.537 | 3.524 |
| ATOM | 38 | HO3 | NAG | 20 | 4.614 | 31.942 | 16.954 | +0.09 | +0.37 | +0.424 | 3.524 |
| ATOM | 39 | O5 | MAN | 20 | 3.603 | 29.163 | 19.820 | +0.15 | −0.18 | −0.227 | 3.524 |
| ATOM | 40 | C5 | MAN | 20 | 2.463 | 28.362 | 19.491 | −0.32 | +0.08 | +0.113 | 3.524 |
| ATOM | 41 | C6 | MAN | 20 | 1.242 | 28.943 | 20.216 | −0.32 | +0.08 | +0.113 | 3.524 |
| ATOM | 42 | O6 | MAN | 20 | 0.246 | 27.926 | 20.498 | −0.03 | −0.15 | −0.227 | 3.524 |
| ATOM | 43 | C1 | MAN | 20 | −0.840 | 28.352 | 21.296 | −0.41 | +0.19 | +0.227 | 3.524 |
| ATOM | 44 | O5 | MAN | 20 | −0.638 | 29.608 | 21.968 | +0.13 | −0.22 | −0.227 | 3.524 |
| ATOM | 45 | C5 | MAN | 20 | −0.703 | 30.787 | 21.124 | −0.19 | +0.11 | +0.113 | 3.524 |
| ATOM | 46 | C6 | MAN | 20 | −0.379 | 32.012 | 21.975 | −0.08 | +0.10 | +0.113 | 3.524 |
| ATOM | 47 | O6 | MAN | 20 | 0.194 | 33.055 | 21.162 | +0.19 | −0.18 | −0.227 | 3.524 |
| ATOM | 48 | C1 | MAN | 20 | 1.475 | 33.444 | 21.589 | −0.06 | +0.16 | +0.227 | 3.524 |
| ATOM | 49 | O5 | MAN | 20 | 2.395 | 33.596 | 20.505 | +0.19 | −0.16 | −0.227 | 3.524 |
| ATOM | 50 | C5 | MAN | 20 | 2.004 | 34.582 | 19.520 | −0.25 | +0.07 | +0.113 | 3.524 |
| ATOM | 51 | C6 | MAN | 20 | 3.087 | 34.615 | 18.462 | −0.33 | +0.08 | +0.113 | 3.524 |
| ATOM | 52 | O6 | MAN | 20 | 3.762 | 35.865 | 18.453 | +0.10 | −0.30 | −0.537 | 3.524 |
| ATOM | 53 | H6 | MAN | 20 | 3.088 | 36.621 | 18.638 | +0.09 | +0.18 | +0.424 | 3.524 |
| ATOM | 54 | C4 | MAN | 20 | 1.814 | 35.958 | 20.169 | −0.18 | +0.05 | +0.113 | 3.524 |
| ATOM | 55 | O4 | MAN | 20 | 1.218 | 36.825 | 19.219 | +0.24 | −0.01 | −0.537 | 3.524 |
| ATOM | 56 | HO4 | MAN | 20 | 0.521 | 37.420 | 19.687 | −0.01 | +0.00 | +0.424 | 3.524 |
| ATOM | 57 | C3 | MAN | 20 | 0.927 | 35.888 | 21.418 | −0.09 | +0.05 | +0.113 | 3.524 |
| ATOM | 58 | O3 | MAN | 20 | 0.976 | 37.130 | 22.106 | +0.21 | −0.25 | −0.537 | 3.524 |
| ATOM | 59 | HO3 | MAN | 20 | 0.017 | 37.447 | 22.311 | +0.12 | +0.19 | +0.424 | 3.524 |
| ATOM | 60 | C2 | MAN | 20 | 1.370 | 34.764 | 22.360 | −0.03 | +0.07 | +0.113 | 3.524 |
| ATOM | 61 | O2 | MAN | 20 | 2.624 | 35.086 | 22.944 | +0.23 | −0.30 | −0.537 | 3.524 |
| ATOM | 62 | HO2 | MAN | 20 | 2.726 | 36.110 | 22.996 | +0.12 | +0.23 | +0.424 | 3.524 |
| ATOM | 63 | C4 | MAN | 20 | −2.066 | 30.865 | 20.429 | −0.29 | +0.13 | +0.113 | 3.524 |
| ATOM | 64 | O4 | MAN | 20 | −2.129 | 32.024 | 19.610 | +0.07 | −0.64 | −0.537 | 3.524 |
| ATOM | 65 | HO4 | MAN | 20 | −2.606 | 32.781 | 20.120 | +0.11 | +0.43 | +0.424 | 3.524 |
| ATOM | 66 | C3 | MAN | 20 | −2.194 | 29.603 | 19.573 | −0.16 | +0.12 | +0.113 | 3.524 |
| ATOM | 67 | O3 | MAN | 20 | −3.376 | 29.613 | 18.726 | −0.05 | −0.25 | −0.227 | 3.524 |
| ATOM | 68 | C1 | BDG | 20 | −4.638 | 29.370 | 19.350 | −0.56 | +0.24 | +0.227 | 3.524 |
| ATOM | 69 | C2 | BDG | 20 | −5.726 | 29.162 | 18.270 | −0.51 | +0.11 | +0.113 | 3.524 |
| ATOM | 70 | O22 | BDG | 20 | −6.942 | 28.766 | 18.908 | +0.02 | −0.13 | −0.113 | 3.524 |
| ATOM | 71 | C3 | BDG | 20 | −5.985 | 30.469 | 17.483 | −0.57 | +0.14 | +0.113 | 3.524 |
| ATOM | 72 | O3 | BDG | 20 | −7.077 | 30.295 | 16.616 | −0.31 | −0.95 | −0.537 | 3.524 |
| ATOM | 73 | HO3 | BDG | 20 | −7.185 | 31.129 | 16.122 | +0.05 | +0.62 | +0.424 | 3.524 |
| ATOM | 74 | C4 | BDG | 20 | −6.248 | 31.679 | 18.417 | −0.28 | +0.12 | +0.113 | 3.524 |
| ATOM | 75 | O4 | BDG | 20 | −6.308 | 32.856 | 17.648 | +0.13 | −0.53 | −0.537 | 3.524 |
| ATOM | 76 | HO4 | BDG | 20 | −6.499 | 33.590 | 18.260 | +0.11 | +0.38 | +0.424 | 3.524 |
| ATOM | 77 | C5 | BDG | 20 | −5.145 | 31.785 | 19.512 | −0.24 | +0.13 | +0.113 | 3.524 |
| ATOM | 78 | C6 | BDG | 20 | −5.444 | 32.858 | 20.599 | −0.13 | +0.12 | +0.113 | 3.524 |
| ATOM | 79 | O6 | BDG | 20 | −5.423 | 34.160 | 20.056 | +0.20 | −0.44 | −0.537 | 3.524 |
| ATOM | 80 | H6 | BDG | 20 | −5.604 | 34.763 | 20.779 | +0.12 | +0.32 | +0.424 | 3.524 |
| ATOM | 81 | O | BDG | 20 | −4.984 | 30.511 | 20.186 | −0.03 | −0.33 | −0.227 | 3.524 |
| ATOM | 82 | C2 | MAN | 20 | −2.149 | 28.362 | 20.477 | −0.52 | +0.11 | +0.113 | 3.524 |
| ATOM | 83 | O2 | MAN | 20 | −3.259 | 28.380 | 21.364 | +0.07 | −0.75 | −0.537 | 3.524 |
| ATOM | 84 | HO2 | MAN | 20 | −3.372 | 27.450 | 21.793 | +0.04 | +0.51 | +0.424 | 3.524 |
| ATOM | 85 | C4 | MAN | 20 | 2.809 | 26.884 | 19.722 | −0.35 | +0.08 | +0.113 | 3.524 |
| ATOM | 86 | O4 | MAN | 20 | 1.720 | 26.071 | 19.311 | −0.06 | −0.26 | −0.537 | 3.524 |
| ATOM | 87 | HO4 | MAN | 20 | 0.945 | 26.670 | 18.992 | +0.05 | +0.24 | +0.424 | 3.524 |
| TER | | | | | | | | | | | |
| ENDMDL | | | | | | | | | | | |

TABLE 21

| | | | | | | | | | | |
|---|---|---|---|---|---|---|---|---|---|---|
| ATOM | 3508 | N | GLY | 317 | 9.095 | 12.623 | 10.296 | 1.00 | 0.00 | N |
| ATOM | 3509 | CA | GLY | 317 | 8.861 | 13.910 | 9.641 | 1.00 | 0.00 | C |
| ATOM | 3510 | C | GLY | 317 | 9.796 | 15.061 | 10.005 | 1.00 | 0.00 | C |
| ATOM | 3511 | O | GLY | 317 | 9.382 | 16.088 | 10.594 | 1.00 | 0.00 | O |
| ATOM | 3512 | H | GLY | 317 | 8.455 | 12.320 | 11.044 | 1.00 | 0.00 | H |
| ATOM | 3513 | 1HA | GLY | 317 | 7.853 | 14.233 | 9.902 | 1.00 | 0.00 | H |
| ATOM | 3514 | 2HA | GLY | 317 | 8.961 | 13.755 | 8.566 | 1.00 | 0.00 | H |
| ATOM | 3515 | N | ARG | 318G | 11.062 | 14.887 | 9.623 | 1.00 | 0.00 | N |
| ATOM | 3516 | CA | ARG | 318G | 12.138 | 15.853 | 9.839 | 1.00 | 0.00 | C |
| ATOM | 3517 | C | ARG | 318G | 11.754 | 17.241 | 9.327 | 1.00 | 0.00 | C |
| ATOM | 3518 | O | ARG | 318G | 11.783 | 18.238 | 10.066 | 1.00 | 0.00 | O |
| ATOM | 3519 | CB | ARG | 318G | 13.382 | 15.375 | 9.089 | 1.00 | 0.00 | C |
| ATOM | 3520 | CG | ARG | 318G | 13.733 | 13.931 | 9.383 | 1.00 | 0.00 | C |
| ATOM | 3521 | CD | ARG | 318G | 14.989 | 13.492 | 8.629 | 1.00 | 0.00 | C |
| ATOM | 3522 | NE | ARG | 318G | 15.768 | 12.530 | 9.409 | 1.00 | 0.00 | N |
| ATOM | 3523 | CZ | ARG | 318G | 16.349 | 12.827 | 10.567 | 1.00 | 0.00 | C |
| ATOM | 3524 | NH1 | ARG | 318G | 16.228 | 14.053 | 11.061 | 1.00 | 0.00 | N |
| ATOM | 3525 | NH2 | ARG | 318G | 17.049 | 11.912 | 11.228 | 1.00 | 0.00 | N |
| ATOM | 3526 | H | ARG | 318G | 11.298 | 14.008 | 9.140 | 1.00 | 0.00 | H |
| ATOM | 3527 | HA | ARG | 318G | 12.379 | 15.950 | 10.897 | 1.00 | 0.00 | H |
| ATOM | 3528 | HO | ARG | 318G | 12.571 | 18.844 | 9.795 | 1.00 | 0.00 | H |
| ATOM | 3529 | 1HB | ARG | 318G | 14.274 | 15.950 | 9.333 | 1.00 | 0.00 | H |
| ATOM | 3530 | 2HB | ARG | 318G | 13.277 | 15.438 | 8.006 | 1.00 | 0.00 | H |
| ATOM | 3531 | 1HG | ARG | 318G | 12.928 | 13.255 | 9.092 | 1.00 | 0.00 | H |
| ATOM | 3532 | 2HG | ARG | 318G | 13.921 | 13.771 | 10.444 | 1.00 | 0.00 | H |
| ATOM | 3533 | 1HD | ARG | 318G | 15.637 | 14.341 | 8.411 | 1.00 | 0.00 | H |
| ATOM | 3534 | 2HD | ARG | 318G | 14.736 | 13.018 | 7.680 | 1.00 | 0.00 | H |
| ATOM | 3535 | HE | ARG | 318G | 15.871 | 11.573 | 9.041 | 1.00 | 0.00 | H |
| ATOM | 3536 | 1HH1 | ARG | 318G | 16.674 | 14.293 | 11.957 | 1.00 | 0.00 | H |
| ATOM | 3537 | 2HH1 | ARG | 318G | 15.687 | 14.764 | 10.547 | 1.00 | 0.00 | H |
| ATOM | 3538 | 1HH2 | ARG | 318G | 17.495 | 12.153 | 12.124 | 1.00 | 0.00 | H |
| ATOM | 3539 | 2HH2 | ARG | 318G | 17.145 | 10.961 | 10.844 | 1.00 | 0.00 | H |
| ATOM | 3540 | N | LYS | 319S | 11.404 | 17.279 | 8.045 | 1.00 | 0.00 | N |
| ATOM | 3541 | CA | LYS | 319S | 10.993 | 18.499 | 7.353 | 1.00 | 0.00 | C |
| ATOM | 3542 | C | LYS | 319S | 9.467 | 18.503 | 7.312 | 1.00 | 0.00 | C |
| ATOM | 3543 | O | LYS | 319S | 8.849 | 17.531 | 6.874 | 1.00 | 0.00 | O |
| ATOM | 3544 | CB | LYS | 319S | 11.571 | 18.496 | 5.924 | 1.00 | 0.00 | C |
| ATOM | 3545 | CG | LYS | 319S | 10.933 | 19.481 | 4.946 | 1.00 | 0.00 | C |
| ATOM | 3546 | CD | LYS | 319S | 11.331 | 20.919 | 5.226 | 1.00 | 0.00 | C |
| ATOM | 3547 | CE | LYS | 319S | 10.614 | 21.862 | 4.274 | 1.00 | 0.00 | C |
| ATOM | 3548 | NZ | LYS | 319S | 9.133 | 21.724 | 4.420 | 1.00 | 0.00 | N |
| ATOM | 3549 | H | LYS | 319S | 11.423 | 16.399 | 7.509 | 1.00 | 0.00 | H |
| ATOM | 3550 | HA | LYS | 319S | 11.381 | 19.341 | 7.925 | 1.00 | 0.00 | H |
| ATOM | 3551 | 1HB | LYS | 319S | 11.433 | 17.497 | 5.509 | 1.00 | 0.00 | H |
| ATOM | 3552 | 2HB | LYS | 319S | 12.629 | 18.749 | 5.989 | 1.00 | 0.00 | H |
| ATOM | 3553 | 1HG | LYS | 319S | 9.843 | 19.454 | 4.977 | 1.00 | 0.00 | H |
| ATOM | 3554 | 2HG | LYS | 319S | 11.213 | 19.283 | 3.911 | 1.00 | 0.00 | H |
| ATOM | 3555 | 1HD | LYS | 319S | 12.405 | 21.047 | 5.097 | 1.00 | 0.00 | H |
| ATOM | 3556 | 2HD | LYS | 319S | 11.069 | 21.193 | 6.248 | 1.00 | 0.00 | H |
| ATOM | 3557 | 1HE | LYS | 319S | 10.882 | 21.636 | 3.241 | 1.00 | 0.00 | H |
| ATOM | 3558 | 2HE | LYS | 319S | 10.887 | 22.895 | 4.485 | 1.00 | 0.00 | H |
| ATOM | 3559 | 1HZ | LYS | 319S | 8.703 | 22.659 | 4.458 | 1.00 | 0.00 | H |
| ATOM | 3560 | 2HZ | LYS | 319S | 8.755 | 21.205 | 3.615 | 1.00 | 0.00 | H |
| ATOM | 3561 | N | GLY | 320 | 8.866 | 19.590 | 7.782 | 1.00 | 0.00 | N |
| ATOM | 3562 | CA | GLY | 320 | 7.420 | 19.697 | 7.784 | 1.00 | 0.00 | C |
| ATOM | 3563 | C | GLY | 320 | 6.964 | 21.130 | 7.951 | 1.00 | 0.00 | C |
| ATOM | 3564 | O | GLY | 320 | 7.638 | 22.068 | 7.492 | 1.00 | 0.00 | O |
| ATOM | 3565 | H | GLY | 320 | 9.434 | 20.366 | 8.149 | 1.00 | 0.00 | H |
| ATOM | 3566 | 1HA | GLY | 320 | 6.973 | 19.122 | 8.595 | 1.00 | 0.00 | H |
| ATOM | 3567 | 2HA | GLY | 320 | 6.986 | 19.329 | 6.854 | 1.00 | 0.00 | H |
| ATOM | 3568 | N | VAL | 321 | 5.809 | 21.317 | 8.582 | 1.00 | 0.00 | N |
| ATOM | 3569 | CA | VAL | 321 | 5.312 | 22.665 | 8.817 | 1.00 | 0.00 | C |
| ATOM | 3570 | C | VAL | 321 | 6.254 | 23.296 | 9.830 | 1.00 | 0.00 | C |
| ATOM | 3571 | O | VAL | 321 | 6.976 | 24.247 | 9.512 | 1.00 | 0.00 | O |
| ATOM | 3572 | CB | VAL | 321 | 3.883 | 22.646 | 9.373 | 1.00 | 0.00 | C |
| ATOM | 3573 | CG1 | VAL | 321 | 3.473 | 24.040 | 9.841 | 1.00 | 0.00 | C |
| ATOM | 3574 | CG2 | VAL | 321 | 2.942 | 22.152 | 8.294 | 1.00 | 0.00 | C |
| ATOM | 3575 | H | VAL | 321 | 5.263 | 20.505 | 8.905 | 1.00 | 0.00 | H |
| ATOM | 3576 | HA | VAL | 321 | 5.337 | 23.172 | 7.852 | 1.00 | 0.00 | H |
| ATOM | 3577 | HO | VAL | 321 | 7.924 | 24.099 | 9.886 | 1.00 | 0.00 | H |
| ATOM | 3578 | HB | VAL | 321 | 3.812 | 21.963 | 10.220 | 1.00 | 0.00 | H |
| ATOM | 3579 | 1HG1 | VAL | 321 | 2.456 | 24.007 | 10.232 | 1.00 | 0.00 | H |
| ATOM | 3580 | 2HG1 | VAL | 321 | 4.152 | 24.375 | 10.624 | 1.00 | 0.00 | H |
| ATOM | 3581 | 3HG1 | VAL | 321 | 3.516 | 24.733 | 9.001 | 1.00 | 0.00 | H |
| ATOM | 3582 | 1HG2 | VAL | 321 | 1.922 | 22.134 | 8.680 | 1.00 | 0.00 | H |
| ATOM | 3583 | 2HG2 | VAL | 321 | 2.992 | 22.819 | 7.433 | 1.00 | 0.00 | H |
| ATOM | 3584 | 3HG2 | VAL | 321 | 3.232 | 21.146 | 7.991 | 1.00 | 0.00 | H |
| ATOM | 3585 | N | SER | 322 | 6.255 | 22.759 | 11.047 | 1.00 | 0.00 | N |
| ATOM | 3586 | CA | SER | 322 | 7.144 | 23.272 | 12.083 | 1.00 | 0.00 | C |
| ATOM | 3587 | C | SER | 322 | 8.582 | 23.088 | 11.627 | 1.00 | 0.00 | C |
| ATOM | 3588 | O | SER | 322 | 8.883 | 22.248 | 10.771 | 1.00 | 0.00 | O |
| ATOM | 3589 | CB | SER | 322 | 6.973 | 22.510 | 13.406 | 1.00 | 0.00 | C |
| ATOM | 3590 | OG | SER | 322 | 5.654 | 22.604 | 13.912 | 1.00 | 0.00 | O |
| ATOM | 3591 | H | SER | 322 | 5.622 | 21.974 | 11.259 | 1.00 | 0.00 | H |
| ATOM | 3592 | HA | SER | 322 | 6.929 | 24.329 | 12.235 | 1.00 | 0.00 | H |
| ATOM | 3593 | 1HB | SER | 322 | 7.633 | 22.886 | 14.187 | 1.00 | 0.00 | H |
| ATOM | 3594 | 2HB | SER | 322 | 7.190 | 21.447 | 13.301 | 1.00 | 0.00 | H |
| ATOM | 3595 | HG | SER | 322 | 5.398 | 23.595 | 14.023 | 1.00 | 0.00 | H |
| ATOM | 3596 | N | HIS | 323S | 9.468 | 23.882 | 12.211 | 1.00 | 0.00 | N |
| ATOM | 3597 | CA | HIS | 323S | 10.882 | 23.788 | 11.909 | 1.00 | 0.00 | C |
| ATOM | 3598 | C | HIS | 323S | 11.392 | 22.543 | 12.658 | 1.00 | 0.00 | C |
| ATOM | 3599 | O | HIS | 323S | 10.748 | 22.069 | 13.603 | 1.00 | 0.00 | O |
| ATOM | 3600 | CB | HIS | 323S | 11.577 | 25.075 | 12.358 | 1.00 | 0.00 | C |
| ATOM | 3601 | CG | HIS | 323S | 10.923 | 26.313 | 11.822 | 1.00 | 0.00 | C |
| ATOM | 3602 | ND1 | HIS | 323S | 10.671 | 26.494 | 10.477 | 1.00 | 0.00 | N |
| ATOM | 3603 | CD2 | HIS | 323S | 10.432 | 27.410 | 12.448 | 1.00 | 0.00 | C |
| ATOM | 3604 | CE1 | HIS | 323S | 10.052 | 27.649 | 10.299 | 1.00 | 0.00 | C |
| ATOM | 3605 | NE2 | HIS | 323S | 9.894 | 28.225 | 11.478 | 1.00 | 0.00 | N |
| ATOM | 3606 | H | HIS | 323S | 9.144 | 24.581 | 12.894 | 1.00 | 0.00 | H |
| ATOM | 3607 | HA | HIS | 323S | 10.950 | 23.686 | 10.826 | 1.00 | 0.00 | H |
| ATOM | 3608 | 1HB | HIS | 323S | 12.618 | 25.131 | 12.040 | 1.00 | 0.00 | H |
| ATOM | 3609 | 2HB | HIS | 323S | 11.592 | 25.195 | 13.441 | 1.00 | 0.00 | H |
| ATOM | 3610 | HD1 | HIS | 323S | 10.925 | 25.833 | 9.729 | 1.00 | 0.00 | H |
| ATOM | 3611 | HD2 | HIS | 323S | 10.458 | 27.609 | 13.519 | 1.00 | 0.00 | H |
| ATOM | 3612 | HE1 | HIS | 323S | 9.727 | 28.056 | 9.342 | 1.00 | 0.00 | H |
| ATOM | 3613 | N | GLY | 324 | 12.534 | 22.013 | 12.231 | 1.00 | 0.00 | N |
| ATOM | 3614 | CA | GLY | 324 | 13.054 | 20.799 | 12.833 | 1.00 | 0.00 | C |
| ATOM | 3615 | C | GLY | 324 | 13.766 | 20.773 | 14.178 | 1.00 | 0.00 | C |
| ATOM | 3616 | O | GLY | 324 | 14.357 | 19.738 | 14.502 | 1.00 | 0.00 | O |
| ATOM | 3617 | H | GLY | 324 | 13.053 | 22.467 | 11.467 | 1.00 | 0.00 | H |
| ATOM | 3618 | 1HA | GLY | 324 | 13.786 | 20.403 | 12.129 | 1.00 | 0.00 | H |
| ATOM | 3619 | 2HA | GLY | 324 | 12.198 | 20.137 | 12.965 | 1.00 | 0.00 | H |
| ATOM | 3620 | N | GLN | 325 | 13.728 | 21.843 | 14.973 | 1.00 | 0.00 | N |
| ATOM | 3621 | CA | GLN | 325 | 14.439 | 21.791 | 16.253 | 1.00 | 0.00 | C |
| ATOM | 3622 | C | GLN | 325 | 13.811 | 20.848 | 17.271 | 1.00 | 0.00 | C |
| ATOM | 3623 | O | GLN | 325 | 14.502 | 20.013 | 17.863 | 1.00 | 0.00 | O |
| ATOM | 3624 | CB | GLN | 325 | 14.596 | 23.177 | 16.878 | 1.00 | 0.00 | C |
| ATOM | 3625 | CG | GLN | 325 | 15.436 | 23.139 | 18.156 | 1.00 | 0.00 | C |
| ATOM | 3626 | CD | GLN | 325 | 16.160 | 24.449 | 18.447 | 1.00 | 0.00 | C |
| ATOM | 3627 | OE1 | GLN | 325 | 15.533 | 25.478 | 18.719 | 1.00 | 0.00 | O |
| ATOM | 3628 | NE2 | GLN | 325 | 17.492 | 24.415 | 18.384 | 1.00 | 0.00 | N |
| ATOM | 3629 | H | GLN | 325 | 13.209 | 22.688 | 14.693 | 1.00 | 0.00 | H |
| ATOM | 3630 | HA | GLN | 325 | 15.457 | 21.450 | 16.067 | 1.00 | 0.00 | H |
| ATOM | 3631 | HO | GLN | 325 | 14.038 | 19.744 | 18.742 | 1.00 | 0.00 | H |
| ATOM | 3632 | 1HB | GLN | 325 | 13.635 | 23.616 | 17.146 | 1.00 | 0.00 | H |
| ATOM | 3633 | 2HB | GLN | 325 | 15.084 | 23.878 | 16.201 | 1.00 | 0.00 | H |
| ATOM | 3634 | 1HG | GLN | 325 | 16.217 | 22.379 | 18.140 | 1.00 | 0.00 | H |
| ATOM | 3635 | 2HG | GLN | 325 | 14.853 | 22.930 | 19.053 | 1.00 | 0.00 | H |
| ATOM | 3636 | HE1 | GLN | 325 | 16.167 | 26.288 | 18.677 | 1.00 | 0.00 | H |
| ATOM | 3637 | 1HE2 | GLN | 325 | 17.974 | 23.534 | 18.153 | 1.00 | 0.00 | H |
| ATOM | 3638 | 2HE2 | GLN | 325 | 18.038 | 25.269 | 18.565 | 1.00 | 0.00 | H |
| ATOM | 3639 | N | PHE | 326 | 12.509 | 20.976 | 17.485 | 1.00 | 0.00 | N |
| ATOM | 3640 | CA | PHE | 326 | 11.829 | 20.109 | 18.428 | 1.00 | 0.00 | C |
| ATOM | 3641 | C | PHE | 326 | 11.946 | 18.659 | 17.964 | 1.00 | 0.00 | C |
| ATOM | 3642 | O | PHE | 326 | 12.147 | 17.757 | 18.786 | 1.00 | 0.00 | O |
| ATOM | 3643 | CB | PHE | 326 | 10.357 | 20.511 | 18.564 | 1.00 | 0.00 | C |
| ATOM | 3644 | CG | PHE | 326 | 9.642 | 19.801 | 19.672 | 1.00 | 0.00 | C |
| ATOM | 3645 | CD1 | PHE | 326 | 10.205 | 19.736 | 20.947 | 1.00 | 0.00 | C |
| ATOM | 3646 | CD2 | PHE | 326 | 8.427 | 19.169 | 19.443 | 1.00 | 0.00 | C |
| ATOM | 3647 | CE1 | PHE | 326 | 9.576 | 19.050 | 21.968 | 1.00 | 0.00 | C |
| ATOM | 3648 | CE2 | PHE | 326 | 7.788 | 18.480 | 20.459 | 1.00 | 0.00 | C |
| ATOM | 3649 | CZ | PHE | 326 | 8.361 | 18.416 | 21.724 | 1.00 | 0.00 | C |
| ATOM | 3650 | H | PHE | 326 | 11.976 | 21.696 | 16.977 | 1.00 | 0.00 | H |
| ATOM | 3651 | HA | PHE | 326 | 12.285 | 20.212 | 19.412 | 1.00 | 0.00 | H |
| ATOM | 3652 | 1HB | PHE | 326 | 9.765 | 20.309 | 17.670 | 1.00 | 0.00 | H |
| ATOM | 3653 | 2HB | PHE | 326 | 10.211 | 21.572 | 18.767 | 1.00 | 0.00 | H |
| ATOM | 3654 | HD1 | PHE | 326 | 11.155 | 20.233 | 21.140 | 1.00 | 0.00 | H |
| ATOM | 3655 | HD2 | PHE | 326 | 7.972 | 19.216 | 18.453 | 1.00 | 0.00 | H |
| ATOM | 3656 | HE1 | PHE | 326 | 10.028 | 19.005 | 22.958 | 1.00 | 0.00 | H |
| ATOM | 3657 | HE2 | PHE | 326 | 6.834 | 17.987 | 20.267 | 1.00 | 0.00 | H |
| ATOM | 3658 | HZ | PHE | 326 | 7.859 | 17.870 | 22.523 | 1.00 | 0.00 | H |
| ATOM | 3659 | N | PHE | 327 | 11.847 | 18.437 | 16.653 | 1.00 | 0.00 | N |
| ATOM | 3660 | CA | PHE | 327 | 11.964 | 17.086 | 16.092 | 1.00 | 0.00 | C |
| ATOM | 3661 | C | PHE | 327 | 13.385 | 16.564 | 16.263 | 1.00 | 0.00 | C |
| ATOM | 3662 | O | PHE | 327 | 13.592 | 15.379 | 16.554 | 1.00 | 0.00 | O |
| ATOM | 3663 | CB | PHE | 327 | 11.633 | 17.061 | 14.584 | 1.00 | 0.00 | C |
| ATOM | 3664 | CG | PHE | 327 | 11.923 | 15.726 | 13.923 | 1.00 | 0.00 | C |
| ATOM | 3665 | CD1 | PHE | 327 | 10.920 | 14.762 | 13.796 | 1.00 | 0.00 | C |
| ATOM | 3666 | CD2 | PHE | 327 | 13.222 | 15.402 | 13.519 | 1.00 | 0.00 | C |
| ATOM | 3667 | CE1 | PHE | 327 | 11.202 | 13.468 | 13.282 | 1.00 | 0.00 | C |
| ATOM | 3668 | CE2 | PHE | 327 | 13.526 | 14.135 | 13.004 | 1.00 | 0.00 | C |
| ATOM | 3669 | CZ | PHE | 327 | 12.512 | 13.171 | 12.886 | 1.00 | 0.00 | C |
| ATOM | 3670 | H | PHE | 327 | 11.684 | 19.232 | 16.019 | 1.00 | 0.00 | H |
| ATOM | 3671 | HA | PHE | 327 | 11.279 | 16.405 | 16.598 | 1.00 | 0.00 | H |
| ATOM | 3672 | 1HB | PHE | 327 | 12.193 | 17.794 | 14.004 | 1.00 | 0.00 | H |
| ATOM | 3673 | 2HB | PHE | 327 | 10.584 | 17.265 | 14.365 | 1.00 | 0.00 | H |
| ATOM | 3674 | HD1 | PHE | 327 | 9.902 | 15.003 | 14.101 | 1.00 | 0.00 | H |
| ATOM | 3675 | HD2 | PHE | 327 | 14.011 | 16.148 | 13.606 | 1.00 | 0.00 | H |
| ATOM | 3676 | HE1 | PHE | 327 | 10.408 | 12.746 | 13.190 | 1.00 | 0.00 | H |
| ATOM | 3677 | HE2 | PHE | 327 | 14.544 | 13.898 | 12.696 | 1.00 | 0.00 | H |
| ATOM | 3678 | HZ | PHE | 327 | 12.740 | 12.181 | 12.489 | 1.00 | 0.00 | H |

TABLE 22

| | | | | | | | |
|---|---|---|---|---|---|---|---|
| ATOM | 3282 | N | SER | 249 | 5.485 | 11.820 | 15.035 |
| ATOM | 3283 | HN | SER | 249 | 5.423 | 12.606 | 15.696 |
| ATOM | 3284 | CA | SER | 249 | 5.843 | 12.092 | 13.657 |
| ATOM | 3285 | HA | SER | 249 | 5.931 | 11.158 | 13.101 |
| ATOM | 3286 | C | SER | 249 | 7.158 | 12.818 | 13.598 |
| ATOM | 3287 | O | SER | 249 | 7.321 | 13.887 | 14.186 |
| ATOM | 3288 | CB | SER | 249 | 4.844 | 13.002 | 12.927 |
| ATOM | 3289 | HB1 | SER | 249 | 3.840 | 12.580 | 12.989 |
| ATOM | 3290 | HB2 | SER | 249 | 5.123 | 13.096 | 11.877 |
| ATOM | 3291 | OG | SER | 249 | 4.839 | 14.292 | 13.520 |
| ATOM | 3292 | HG | SER | 249 | 5.720 | 14.776 | 13.296 |
| ATOM | 3293 | N | HIS | 250 | 8.134 | 12.245 | 12.861 |
| ATOM | 3294 | HN | HIS | 250 | 7.939 | 11.349 | 12.390 |
| ATOM | 3295 | CA | HIS | 250 | 9.435 | 12.839 | 12.709 |
| ATOM | 3296 | HA | HIS | 250 | 9.863 | 13.073 | 13.683 |
| ATOM | 3297 | C | HIS | 250 | 9.343 | 14.104 | 11.916 |
| ATOM | 3298 | O | HIS | 250 | 9.857 | 15.142 | 12.331 |
| ATOM | 3299 | CB | HIS | 250 | 10.429 | 11.932 | 11.962 |
| ATOM | 3300 | HB1 | HIS | 250 | 11.309 | 12.523 | 11.710 |
| ATOM | 3301 | HB2 | HIS | 250 | 9.943 | 11.563 | 11.058 |
| ATOM | 3302 | CG | HIS | 250 | 10.889 | 10.742 | 12.751 |
| ATOM | 3303 | ND1 | HIS | 250 | 10.132 | 9.614 | 12.972 |
| ATOM | 3304 | HD1 | HIS | 250 | 9.172 | 9.443 | 12.639 |
| ATOM | 3305 | CD2 | HIS | 250 | 12.079 | 10.519 | 13.374 |
| ATOM | 3306 | HD2 | HIS | 250 | 12.911 | 11.222 | 13.391 |
| ATOM | 3307 | CE1 | HIS | 250 | 10.894 | 8.770 | 13.712 |
| ATOM | 3308 | HE1 | HIS | 250 | 10.562 | 7.787 | 14.046 |
| ATOM | 3309 | NE2 | HIS | 250 | 12.084 | 9.276 | 13.981 |
| ATOM | 3310 | HE2 | HIS | 250 | 12.845 | 8.842 | 14.521 |
| ATOM | 3311 | N | LYS | 251 | 8.665 | 14.062 | 10.753 |
| ATOM | 3312 | HN | LYS | 251 | 8.179 | 13.202 | 10.459 |
| ATOM | 3313 | CA | LYS | 251 | 8.632 | 15.236 | 9.933 |
| ATOM | 3314 | HA | LYS | 251 | 9.381 | 15.929 | 10.314 |
| ATOM | 3315 | C | LYS | 251 | 7.267 | 15.818 | 10.027 |
| ATOM | 3316 | O | LYS | 251 | 6.270 | 15.099 | 10.034 |
| ATOM | 3317 | CB | LYS | 251 | 8.960 | 14.968 | 8.449 |
| ATOM | 3318 | HB1 | LYS | 251 | 9.911 | 14.438 | 8.403 |
| ATOM | 3319 | HB2 | LYS | 251 | 9.026 | 15.928 | 7.938 |
| ATOM | 3320 | CG | LYS | 251 | 7.930 | 14.121 | 7.694 |
| ATOM | 3321 | HG1 | LYS | 251 | 7.633 | 13.289 | 8.333 |
| ATOM | 3322 | HG2 | LYS | 251 | 8.392 | 13.752 | 6.778 |
| ATOM | 3323 | CD | LYS | 251 | 6.663 | 14.885 | 7.303 |
| ATOM | 3324 | HD1 | LYS | 251 | 6.176 | 15.376 | 8.145 |
| ATOM | 3325 | HD2 | LYS | 251 | 5.895 | 14.252 | 6.857 |
| ATOM | 3326 | CE | LYS | 251 | 6.913 | 15.995 | 6.279 |
| ATOM | 3327 | HE1 | LYS | 251 | 7.324 | 15.573 | 5.362 |
| ATOM | 3328 | HE2 | LYS | 251 | 7.619 | 16.722 | 6.679 |
| ATOM | 3329 | NZ | LYS | 251 | 5.645 | 16.687 | 5.959 |
| ATOM | 3330 | HZ1 | LYS | 251 | 5.823 | 17.431 | 5.269 |
| ATOM | 3331 | HZ2 | LYS | 251 | 4.973 | 16.011 | 5.567 |
| ATOM | 3332 | HZ3 | LYS | 251 | 5.253 | 17.101 | 6.816 |
| ATOM | 3333 | N | GLU | 252 | 7.195 | 17.157 | 10.115 |
| ATOM | 3334 | HN | GLU | 252 | 8.053 | 17.725 | 10.084 |
| ATOM | 3335 | CA | GLU | 252 | 5.921 | 17.787 | 10.251 |
| ATOM | 3336 | HA | GLU | 252 | 5.229 | 17.236 | 9.613 |
| ATOM | 3337 | C | GLU | 252 | 6.091 | 19.200 | 9.804 |
| ATOM | 3338 | O | GLU | 252 | 7.048 | 19.541 | 9.112 |
| ATOM | 3339 | CB | GLU | 252 | 5.436 | 17.791 | 11.712 |
| ATOM | 3340 | HB1 | GLU | 252 | 4.703 | 18.590 | 11.822 |
| ATOM | 3341 | HB2 | GLU | 252 | 6.299 | 17.965 | 12.354 |
| ATOM | 3342 | CG | GLU | 252 | 4.772 | 16.495 | 12.182 |
| ATOM | 3343 | HG1 | GLU | 252 | 5.499 | 15.694 | 12.050 |
| ATOM | 3344 | HG2 | GLU | 252 | 3.888 | 16.342 | 11.561 |
| ATOM | 3345 | CD | GLU | 252 | 4.404 | 16.684 | 13.647 |
| ATOM | 3346 | OE1 | GLU | 252 | 5.329 | 16.611 | 14.500 |
| ATOM | 3347 | OE2 | GLU | 252 | 3.197 | 16.904 | 13.934 |
| ATOM | 3349 | N | GLU | 253 | 5.118 | 20.052 | 10.161 |
| ATOM | 3350 | HN | GLU | 253 | 4.306 | 19.698 | 10.687 |
| ATOM | 3351 | CA | GLU | 253 | 5.184 | 21.439 | 9.827 |
| ATOM | 3352 | HA | GLU | 253 | 5.300 | 21.500 | 8.745 |
| ATOM | 3353 | C | GLU | 253 | 6.365 | 22.005 | 10.547 |
| ATOM | 3354 | O | GLU | 253 | 7.167 | 22.741 | 9.974 |
| ATOM | 3355 | CB | GLU | 253 | 3.923 | 22.184 | 10.303 |
| ATOM | 3356 | HB1 | GLU | 253 | 3.866 | 22.096 | 11.388 |
| ATOM | 3357 | HB2 | GLU | 253 | 3.056 | 21.720 | 9.832 |
| ATOM | 3358 | CG | GLU | 253 | 3.888 | 23.673 | 9.961 |
| ATOM | 3359 | HG1 | GLU | 253 | 2.855 | 24.019 | 10.009 |
| ATOM | 3360 | HG2 | GLU | 253 | 4.283 | 23.811 | 8.954 |
| ATOM | 3361 | CD | GLU | 253 | 4.743 | 24.427 | 10.969 |
| ATOM | 3362 | OE1 | GLU | 253 | 4.525 | 24.234 | 12.194 |
| ATOM | 3363 | OE2 | GLU | 253 | 5.632 | 25.200 | 10.523 |
| ATOM | 3365 | N | ARG | 254 | 6.512 | 21.644 | 11.836 |
| ATOM | 3366 | HN | ARG | 254 | 5.839 | 20.988 | 12.260 |
| ATOM | 3367 | CA | ARG | 254 | 7.590 | 22.159 | 12.625 |
| ATOM | 3368 | HA | ARG | 254 | 7.638 | 23.237 | 12.473 |
| ATOM | 3369 | C | ARG | 254 | 8.851 | 21.506 | 12.173 |
| ATOM | 3370 | O | ARG | 254 | 8.833 | 20.571 | 11.373 |
| ATOM | 3371 | CB | ARG | 254 | 7.432 | 21.887 | 14.130 |
| ATOM | 3372 | HB1 | ARG | 254 | 6.524 | 22.315 | 14.554 |
| ATOM | 3373 | HB2 | ARG | 254 | 8.247 | 22.289 | 14.731 |
| ATOM | 3374 | CG | ARG | 254 | 7.374 | 20.398 | 14.476 |
| ATOM | 3375 | HG1 | ARG | 254 | 7.862 | 19.758 | 13.740 |
| ATOM | 3376 | HG2 | ARG | 254 | 6.359 | 20.007 | 14.559 |
| ATOM | 3377 | CD | ARG | 254 | 8.042 | 20.050 | 15.808 |
| ATOM | 3378 | HD1 | ARG | 254 | 7.880 | 18.987 | 15.989 |
| ATOM | 3379 | HD2 | ARG | 254 | 7.573 | 20.659 | 16.580 |
| ATOM | 3380 | NE | ARG | 254 | 9.493 | 20.362 | 15.679 |
| ATOM | 3381 | HE | ARG | 254 | 10.014 | 20.055 | 14.845 |
| ATOM | 3382 | CZ | ARG | 254 | 10.130 | 21.055 | 16.668 |
| ATOM | 3383 | NH1 | ARG | 254 | 9.437 | 21.460 | 17.772 |
| ATOM | 3384 | HH1 | ARG | 254 | 9.917 | 21.982 | 18.518 |
| ATOM | 3385 | HH1 | ARG | 254 | 8.433 | 21.242 | 17.858 |
| ATOM | 3386 | NH2 | ARG | 254 | 11.459 | 21.344 | 16.552 |
| ATOM | 3387 | HH2 | ARG | 254 | 11.939 | 21.866 | 17.298 |
| ATOM | 3388 | HH2 | ARG | 254 | 11.981 | 21.039 | 15.718 |
| ATOM | 3389 | N | TRP | 255 | 9.990 | 22.014 | 12.678 |
| ATOM | 3390 | HN | TRP | 255 | 9.938 | 22.787 | 13.355 |
| ATOM | 3391 | CA | TRP | 255 | 11.271 | 21.504 | 12.295 |
| ATOM | 3392 | HA | TRP | 255 | 11.288 | 21.348 | 11.216 |
| ATOM | 3393 | C | TRP | 255 | 11.514 | 20.205 | 12.995 |
| ATOM | 3394 | O | TRP | 255 | 10.968 | 19.935 | 14.064 |
| ATOM | 3395 | CB | TRP | 255 | 12.436 | 22.449 | 12.636 |
| ATOM | 3396 | HB1 | TRP | 255 | 13.371 | 21.920 | 12.453 |
| ATOM | 3397 | HB2 | TRP | 255 | 12.354 | 22.730 | 13.686 |
| ATOM | 3398 | CG | TRP | 255 | 12.456 | 23.719 | 11.819 |
| ATOM | 3399 | CD1 | TRP | 255 | 11.606 | 24.131 | 10.834 |
| ATOM | 3400 | HD1 | TRP | 255 | 10.739 | 23.571 | 10.481 |
| ATOM | 3401 | CD2 | TRP | 255 | 13.443 | 24.751 | 11.962 |
| ATOM | 3402 | NE1 | TRP | 255 | 12.004 | 25.355 | 10.353 |
| ATOM | 3403 | HE1 | TRP | 255 | 11.537 | 25.888 | 9.605 |
| ATOM | 3404 | CE2 | TRP | 255 | 13.133 | 25.749 | 11.039 |
| ATOM | 3405 | CE3 | TRP | 255 | 14.520 | 24.858 | 12.794 |
| ATOM | 3406 | HE3 | TRP | 255 | 14.760 | 24.075 | 13.514 |
| ATOM | 3407 | CZ2 | TRP | 255 | 13.900 | 26.875 | 10.933 |
| ATOM | 3408 | HZ2 | TRP | 255 | 13.661 | 27.656 | 10.211 |
| ATOM | 3409 | CZ3 | TRP | 255 | 15.291 | 25.994 | 12.687 |
| ATOM | 3410 | HZ3 | TRP | 255 | 16.158 | 26.114 | 13.336 |
| ATOM | 3411 | CH2 | TRP | 255 | 14.987 | 26.983 | 11.774 |
| ATOM | 3412 | HH2 | TRP | 255 | 15.619 | 27.868 | 11.716 |
| ATOM | 3413 | N | LYS | 256 | 12.348 | 19.363 | 12.356 |
| ATOM | 3414 | HN | LYS | 256 | 12.749 | 19.698 | 11.468 |
| ATOM | 3415 | CA | LYS | 256 | 12.738 | 18.049 | 12.778 |
| ATOM | 3416 | HA | LYS | 256 | 11.871 | 17.450 | 13.059 |
| ATOM | 3417 | C | LYS | 256 | 13.653 | 18.108 | 13.963 |
| ATOM | 3418 | O | LYS | 256 | 13.708 | 17.164 | 14.745 |
| ATOM | 3419 | CB | LYS | 256 | 13.468 | 17.271 | 11.668 |
| ATOM | 3420 | HB1 | LYS | 256 | 14.412 | 17.722 | 11.364 |
| ATOM | 3421 | HB2 | LYS | 256 | 12.893 | 17.177 | 10.746 |
| ATOM | 3422 | CG | LYS | 256 | 13.825 | 15.836 | 12.055 |
| ATOM | 3423 | HG1 | LYS | 256 | 13.005 | 15.297 | 12.531 |
| ATOM | 3424 | HG2 | LYS | 256 | 14.656 | 15.769 | 12.756 |
| ATOM | 3425 | CD | LYS | 256 | 14.236 | 14.966 | 10.863 |
| ATOM | 3426 | HD1 | LYS | 256 | 13.544 | 15.020 | 10.022 |
| ATOM | 3427 | HD2 | LYS | 256 | 14.307 | 13.904 | 11.100 |
| ATOM | 3428 | CE | LYS | 256 | 15.601 | 15.333 | 10.276 |
| ATOM | 3429 | HE1 | LYS | 256 | 16.375 | 15.222 | 11.035 |
| ATOM | 3430 | HE2 | LYS | 256 | 15.591 | 16.365 | 9.927 |
| ATOM | 3431 | NZ | LYS | 256 | 15.917 | 14.444 | 9.135 |
| ATOM | 3432 | HZ1 | LYS | 256 | 16.836 | 14.697 | 8.746 |
| ATOM | 3433 | HZ2 | LYS | 256 | 15.938 | 13.465 | 9.457 |
| ATOM | 3434 | HZ3 | LYS | 256 | 15.195 | 14.549 | 8.407 |
| ATOM | 3435 | N | LYS | 257 | 14.401 | 19.212 | 14.134 |
| ATOM | 3436 | HN | LYS | 257 | 14.235 | 20.031 | 13.532 |
| ATOM | 3437 | CA | LYS | 257 | 15.428 | 19.273 | 15.139 |
| ATOM | 3438 | HA | LYS | 257 | 16.232 | 18.567 | 14.932 |
| ATOM | 3439 | C | LYS | 257 | 14.887 | 18.948 | 16.499 |
| ATOM | 3440 | O | LYS | 257 | 15.469 | 18.140 | 17.221 |
| ATOM | 3441 | CB | LYS | 257 | 16.070 | 20.670 | 15.222 |
| ATOM | 3442 | HB1 | LYS | 257 | 15.290 | 21.390 | 15.469 |
| ATOM | 3443 | HB2 | LYS | 257 | 16.512 | 20.901 | 14.253 |
| ATOM | 3444 | CG | LYS | 257 | 17.171 | 20.796 | 16.277 |
| ATOM | 3445 | HG1 | LYS | 257 | 17.899 | 19.986 | 16.234 |
| ATOM | 3446 | HG2 | LYS | 257 | 16.788 | 20.792 | 17.297 |
| ATOM | 3447 | CD | LYS | 257 | 17.983 | 22.087 | 16.147 |
| ATOM | 3448 | HD1 | LYS | 257 | 17.384 | 22.993 | 16.234 |
| ATOM | 3449 | HD2 | LYS | 257 | 18.501 | 22.186 | 15.193 |
| ATOM | 3450 | CE | LYS | 257 | 19.078 | 22.237 | 17.205 |
| ATOM | 3451 | HE1 | LYS | 257 | 19.783 | 21.409 | 17.130 |
| ATOM | 3452 | HE2 | LYS | 257 | 18.635 | 22.236 | 18.201 |
| ATOM | 3453 | NZ | LYS | 257 | 19.806 | 23.510 | 17.002 |
| ATOM | 3454 | HZ1 | LYS | 257 | 20.540 | 23.603 | 17.717 |
| ATOM | 3455 | HZ2 | LYS | 257 | 20.236 | 23.514 | 16.066 |
| ATOM | 3456 | HZ3 | LYS | 257 | 19.148 | 24.298 | 17.080 |
| ATOM | 3457 | N | ARG | 258 | 13.750 | 19.542 | 16.894 |
| ATOM | 3458 | HN | ARG | 258 | 13.236 | 20.166 | 16.255 |
| ATOM | 3459 | CA | ARG | 258 | 13.261 | 19.294 | 18.219 |
| ATOM | 3460 | HA | ARG | 258 | 14.028 | 19.593 | 18.932 |
| ATOM | 3461 | C | ARG | 258 | 12.967 | 17.833 | 18.351 |
| ATOM | 3462 | O | ARG | 258 | 13.325 | 17.200 | 19.343 |
| ATOM | 3463 | CB | ARG | 258 | 11.964 | 20.066 | 18.511 |
| ATOM | 3464 | HB1 | ARG | 258 | 11.177 | 19.669 | 17.869 |
| ATOM | 3465 | HB2 | ARG | 258 | 12.137 | 21.120 | 18.297 |
| ATOM | 3466 | CG | ARG | 258 | 11.483 | 19.957 | 19.958 |
| ATOM | 3467 | HG1 | ARG | 258 | 11.418 | 18.931 | 20.322 |
| ATOM | 3468 | HG2 | ARG | 258 | 10.490 | 20.376 | 20.122 |
| ATOM | 3469 | CD | ARG | 258 | 12.386 | 20.675 | 20.962 |
| ATOM | 3470 | HD1 | ARG | 258 | 12.446 | 21.720 | 20.658 |
| ATOM | 3471 | HD2 | ARG | 258 | 13.362 | 20.192 | 20.925 |
| ATOM | 3472 | NE | ARG | 258 | 11.759 | 20.534 | 22.307 |
| ATOM | 3473 | HE | ARG | 258 | 11.142 | 21.276 | 22.667 |
| ATOM | 3474 | CZ | ARG | 258 | 12.001 | 19.423 | 23.061 |
| ATOM | 3475 | NH1 | ARG | 258 | 12.819 | 18.440 | 22.585 |
| ATOM | 3476 | HH1 | ARG | 258 | 13.001 | 17.601 | 23.154 |
| ATOM | 3477 | HH1 | ARG | 258 | 13.253 | 18.536 | 21.656 |
| ATOM | 3478 | NH2 | ARG | 258 | 11.422 | 19.295 | 24.290 |
| ATOM | 3479 | HH2 | ARG | 258 | 11.603 | 18.456 | 24.860 |
| ATOM | 3480 | HH2 | ARG | 258 | 10.803 | 20.036 | 24.647 |
| ATOM | 3481 | N | TYR | 259 | 12.314 | 17.254 | 17.331 |
| ATOM | 3482 | HN | TYR | 259 | 12.083 | 17.813 | 16.497 |
| ATOM | 3483 | CA | TYR | 259 | 11.931 | 15.875 | 17.376 |
| ATOM | 3484 | HA | TYR | 259 | 11.329 | 15.699 | 18.268 |
| ATOM | 3485 | C | TYR | 259 | 13.163 | 15.024 | 17.420 |
| ATOM | 3486 | O | TYR | 259 | 13.263 | 14.099 | 18.225 |
| ATOM | 3487 | CB | TYR | 259 | 11.132 | 15.462 | 16.128 |
| ATOM | 3488 | HB1 | TYR | 259 | 11.772 | 15.587 | 15.255 |
| ATOM | 3489 | HB2 | TYR | 259 | 10.254 | 16.104 | 16.056 |
| ATOM | 3490 | CG | TYR | 259 | 10.729 | 14.037 | 16.283 |
| ATOM | 3491 | CD1 | TYR | 259 | 9.549 | 13.718 | 16.915 |
| ATOM | 3492 | HD1 | TYR | 259 | 8.912 | 14.514 | 17.301 |
| ATOM | 3493 | CD2 | TYR | 259 | 11.526 | 13.023 | 15.806 |
| ATOM | 3494 | HD2 | TYR | 259 | 12.465 | 13.261 | 15.307 |
| ATOM | 3495 | CE1 | TYR | 259 | 9.163 | 12.407 | 17.065 |
| ATOM | 3496 | HE1 | TYR | 259 | 8.224 | 12.167 | 17.565 |
| ATOM | 3497 | CE2 | TYR | 259 | 11.145 | 11.710 | 15.954 |
| ATOM | 3498 | HE2 | TYR | 259 | 11.782 | 10.912 | 15.571 |
| ATOM | 3499 | CZ | TYR | 259 | 9.963 | 11.400 | 16.583 |
| ATOM | 3500 | OH | TYR | 259 | 9.572 | 10.053 | 16.733 |
| ATOM | 3501 | HH | TYR | 259 | 8.549 | 10.002 | 16.846 |
| ATOM | 3502 | N | GLU | 260 | 14.148 | 15.339 | 16.558 |
| ATOM | 3503 | HN | GLU | 260 | 14.041 | 16.173 | 15.963 |
| ATOM | 3504 | CA | GLU | 260 | 15.342 | 14.551 | 16.439 |
| ATOM | 3505 | HA | GLU | 260 | 15.077 | 13.518 | 16.210 |
| ATOM | 3506 | C | GLU | 260 | 16.099 | 14.594 | 17.721 |
| ATOM | 3507 | O | GLU | 260 | 16.596 | 13.570 | 18.186 |
| ATOM | 3508 | CB | GLU | 260 | 16.279 | 15.034 | 15.315 |
| ATOM | 3509 | HB1 | GLU | 260 | 15.729 | 15.006 | 14.374 |
| ATOM | 3510 | HB2 | GLU | 260 | 17.140 | 14.366 | 15.275 |
| ATOM | 3511 | CG | GLU | 260 | 16.801 | 16.459 | 15.509 |
| ATOM | 3512 | HG1 | GLU | 260 | 17.357 | 16.488 | 16.445 |
| ATOM | 3513 | HG2 | GLU | 260 | 15.942 | 17.129 | 15.544 |
| ATOM | 3514 | CD | GLU | 260 | 17.704 | 16.793 | 14.329 |
| ATOM | 3515 | OE1 | GLU | 260 | 17.854 | 15.919 | 13.433 |
| ATOM | 3516 | OE2 | GLU | 260 | 18.260 | 17.924 | 14.312 |

TABLE 23

| | | | | | | | | | | | | |
|---|---|---|---|---|---|---|---|---|---|---|---|---|
| ATOM | 3905 | N | PHE | 569 | −12.735 | −0.114 | −4.694 | 0.00 | 0.00 | −0.650 | 9.00 | −17.40 |
| ATOM | 3906 | HN | PHE | 569 | −12.725 | 0.834 | −4.292 | 0.00 | 0.00 | 0.439 | 0.00 | 0.00 |
| ATOM | 3907 | CA | PHE | 569 | −11.996 | −0.393 | −5.884 | 0.00 | 0.00 | 0.158 | 9.40 | 4.00 |
| ATOM | 3908 | HA | PHE | 569 | −12.674 | −0.839 | −6.611 | 0.00 | 0.00 | 0.053 | 0.00 | 0.00 |
| ATOM | 3909 | C | PHE | 569 | −10.861 | −1.330 | −5.692 | 0.00 | 0.00 | 0.396 | 9.82 | 4.00 |
| ATOM | 3910 | O | PHE | 569 | −10.731 | −2.262 | −6.480 | 0.00 | 0.00 | −0.396 | 8.17 | −17.40 |
| ATOM | 3911 | CB | PHE | 569 | −11.533 | 0.861 | −6.631 | 0.00 | 0.00 | −0.106 | 12.77 | 4.00 |
| ATOM | 3912 | HB1 | PHE | 569 | −10.749 | 0.631 | −7.352 | 0.00 | 0.00 | 0.053 | 0.00 | 0.00 |
| ATOM | 3913 | HB2 | PHE | 569 | −11.135 | 1.610 | −5.947 | 0.00 | 0.00 | 0.053 | 0.00 | 0.00 |
| ATOM | 3914 | CG | PHE | 569 | −12.795 | 1.296 | −7.281 | 0.00 | 0.00 | 0.000 | 7.26 | 0.60 |
| ATOM | 3915 | CD1 | PHE | 569 | −13.156 | 0.777 | −8.502 | 0.00 | 0.00 | −0.127 | 10.80 | 0.60 |
| ATOM | 3916 | HD1 | PHE | 569 | −12.506 | 0.054 | −8.995 | 0.00 | 0.00 | 0.127 | 0.00 | 0.00 |
| ATOM | 3917 | CD2 | PHE | 569 | −13.632 | 2.187 | −6.659 | 0.00 | 0.00 | −0.127 | 10.80 | 0.60 |
| ATOM | 3918 | HD2 | PHE | 569 | −13.364 | 2.589 | −5.682 | 0.00 | 0.00 | 0.127 | 0.00 | 0.00 |
| ATOM | 3919 | CE1 | PHE | 569 | −14.328 | 1.162 | −9.106 | 0.00 | 0.00 | −0.127 | 10.80 | 0.60 |
| ATOM | 3920 | HE1 | PHE | 569 | −14.602 | 0.750 | −10.077 | 0.00 | 0.00 | 0.127 | 0.00 | 0.00 |
| ATOM | 3921 | CE2 | PHE | 569 | −14.806 | 2.577 | −7.259 | 0.00 | 0.00 | −0.127 | 10.80 | 0.60 |
| ATOM | 3922 | HE2 | PHE | 569 | −15.461 | 3.292 | −6.761 | 0.00 | 0.00 | 0.127 | 0.00 | 0.00 |
| ATOM | 3923 | CZ | PHE | 569 | −15.153 | 2.066 | −8.486 | 0.00 | 0.00 | −0.127 | 10.80 | 0.60 |
| ATOM | 3924 | HZ | PHE | 569 | −16.081 | 2.378 | −8.966 | 0.00 | 0.00 | 0.127 | 0.00 | 0.00 |
| ATOM | 3925 | N | ILE | 570 | −10.054 | −1.202 | −4.615 | 0.00 | 0.00 | −0.650 | 9.00 | −17.40 |
| ATOM | 3926 | HN | ILE | 570 | −10.257 | −0.508 | −3.882 | 0.00 | 0.00 | 0.439 | 0.00 | 0.00 |
| ATOM | 3927 | CA | ILE | 570 | −8.900 | −2.064 | −4.520 | 0.00 | 0.00 | 0.158 | 9.40 | 4.00 |
| ATOM | 3928 | HA | ILE | 570 | −8.327 | −1.912 | −5.434 | 0.00 | 0.00 | 0.053 | 0.00 | 0.00 |
| ATOM | 3929 | C | ILE | 570 | −9.422 | −3.452 | −4.403 | 0.00 | 0.00 | 0.396 | 9.82 | 4.00 |
| ATOM | 3930 | O | ILE | 570 | −8.710 | −4.429 | −4.622 | 0.00 | 0.00 | −0.396 | 8.17 | −17.40 |
| ATOM | 3931 | CB | ILE | 570 | −8.024 | −1.914 | −3.301 | 0.00 | 0.00 | −0.053 | 9.40 | 4.00 |
| ATOM | 3932 | HB | ILE | 570 | −7.077 | −2.412 | −3.510 | 0.00 | 0.00 | 0.053 | 0.00 | 0.00 |
| ATOM | 3933 | CG1 | ILE | 570 | −8.549 | −2.696 | −2.074 | 0.00 | 0.00 | −0.106 | 12.77 | 4.00 |
| ATOM | 3934 | HG11 | ILE | 570 | −7.876 | −2.484 | −1.243 | 0.00 | 0.00 | 0.053 | 0.00 | 0.00 |
| ATOM | 3935 | HG12 | ILE | 570 | −8.541 | −3.754 | −2.336 | 0.00 | 0.00 | 0.053 | 0.00 | 0.00 |
| ATOM | 3936 | CG2 | ILE | 570 | −7.818 | −0.411 | −3.056 | 0.00 | 0.00 | −0.159 | 16.15 | 4.00 |
| ATOM | 3937 | HG21 | ILE | 570 | −7.187 | −0.268 | −2.179 | 0.00 | 0.00 | 0.053 | 0.00 | 0.00 |
| ATOM | 3938 | HG22 | ILE | 570 | −7.337 | 0.037 | −3.926 | 0.00 | 0.00 | 0.053 | 0.00 | 0.00 |
| ATOM | 3939 | HG23 | ILE | 570 | −8.784 | 0.066 | −2.890 | 0.00 | 0.00 | 0.053 | 0.00 | 0.00 |
| ATOM | 3940 | CD1 | ILE | 570 | −9.954 | −2.393 | −1.572 | 0.00 | 0.00 | −0.159 | 16.15 | 4.00 |
| ATOM | 3941 | HD11 | ILE | 570 | −10.175 | −3.021 | −0.709 | 0.00 | 0.00 | 0.053 | 0.00 | 0.00 |
| ATOM | 3942 | HD12 | ILE | 570 | −10.019 | −1.344 | −1.284 | 0.00 | 0.00 | 0.053 | 0.00 | 0.00 |
| ATOM | 3943 | HD13 | ILE | 570 | −10.675 | −2.597 | −2.363 | 0.00 | 0.00 | 0.053 | 0.00 | 0.00 |
| ATOM | 3944 | N | GLY | 571 | −10.703 | −3.559 | −4.031 | 0.00 | 0.00 | −0.650 | 9.00 | −17.40 |
| ATOM | 3945 | HN | GLY | 571 | −11.269 | −2.709 | −3.897 | 0.00 | 0.00 | 0.439 | 0.00 | 0.00 |
| ATOM | 3946 | CA | GLY | 571 | −11.287 | −4.831 | −3.820 | 0.00 | 0.00 | 0.105 | 9.40 | 4.00 |
| ATOM | 3947 | HA1 | GLY | 571 | −10.745 | −5.613 | −4.352 | 0.00 | 0.00 | 0.053 | 0.00 | 0.00 |
| ATOM | 3948 | HA2 | GLY | 571 | −12.320 | −4.859 | −4.166 | 0.00 | 0.00 | 0.053 | 0.00 | 0.00 |
| ATOM | 3949 | C | GLY | 571 | −11.178 | −4.965 | −2.354 | 0.00 | 0.00 | 0.396 | 9.82 | 4.00 |
| ATOM | 3950 | O | GLY | 571 | −10.077 | −5.124 | −1.829 | 0.00 | 0.00 | −0.396 | 8.17 | −17.40 |
| ATOM | 3951 | N | ARG+ | 572 | −12.317 | −4.873 | −1.646 | 0.00 | 0.00 | −0.650 | 9.00 | −17.40 |
| ATOM | 3952 | HN | ARG+ | 572 | −13.233 | −4.750 | −2.101 | 0.00 | 0.00 | 0.439 | 0.00 | 0.00 |
| ATOM | 3953 | CA | ARG+ | 572 | −12.183 | −4.956 | −0.232 | 0.00 | 0.00 | 0.158 | 9.40 | 4.00 |
| ATOM | 3954 | HA | ARG+ | 572 | −11.507 | −4.162 | 0.085 | 0.00 | 0.00 | 0.053 | 0.00 | 0.00 |
| ATOM | 3955 | C | ARG+ | 572 | −11.623 | −6.302 | 0.094 | 0.00 | 0.00 | 0.396 | 9.82 | 4.00 |
| ATOM | 3956 | O | ARG+ | 572 | −10.832 | −6.407 | 1.030 | 0.00 | 0.00 | −0.396 | 8.17 | −17.40 |
| ATOM | 3957 | CB | ARG+ | 572 | −13.448 | −4.583 | 0.589 | 0.00 | 0.00 | −0.106 | 12.77 | 4.00 |
| ATOM | 3958 | HB1 | ARG+ | 572 | −13.660 | −3.532 | 0.394 | 0.00 | 0.00 | 0.053 | 0.00 | 0.00 |
| ATOM | 3959 | HB2 | ARG+ | 572 | −13.214 | −4.758 | 1.639 | 0.00 | 0.00 | 0.053 | 0.00 | 0.00 |
| ATOM | 3960 | CG | ARG+ | 572 | −14.750 | −5.336 | 0.314 | 0.00 | 0.00 | −0.106 | 12.77 | 4.00 |
| ATOM | 3961 | HG1 | ARG+ | 572 | −14.704 | −6.393 | 0.577 | 0.00 | 0.00 | 0.053 | 0.00 | 0.00 |
| ATOM | 3962 | HG2 | ARG+ | 572 | −15.051 | −5.315 | −0.733 | 0.00 | 0.00 | 0.053 | 0.00 | 0.00 |
| ATOM | 3963 | CD | ARG+ | 572 | −15.948 | −4.783 | 1.086 | 0.00 | 0.00 | 0.374 | 12.77 | 4.00 |
| ATOM | 3964 | HD1 | ARG+ | 572 | −15.631 | −4.656 | 2.121 | 0.00 | 0.00 | 0.053 | 0.00 | 0.00 |
| ATOM | 3965 | HD2 | ARG+ | 572 | −16.752 | −5.514 | 0.996 | 0.00 | 0.00 | 0.053 | 0.00 | 0.00 |
| ATOM | 3966 | NE | ARG+ | 572 | −16.312 | −3.482 | 0.457 | 0.00 | 0.00 | −0.819 | 9.00 | −24.67 |
| ATOM | 3967 | HE | ARG+ | 572 | −15.775 | −3.134 | −0.350 | 0.00 | 0.00 | 0.407 | 0.00 | 0.00 |
| ATOM | 3968 | CZ | ARG+ | 572 | −17.355 | −2.751 | 0.948 | 0.00 | 0.00 | 0.796 | 6.95 | 4.00 |
| ATOM | 3969 | NH1 | ARG+ | 572 | −18.058 | −3.203 | 2.027 | 0.00 | 0.00 | −0.746 | 9.00 | −24.67 |
| ATOM | 3970 | HH11 | ARG+ | 572 | −18.845 | −2.651 | 2.397 | 0.00 | 0.00 | 0.407 | 0.00 | 0.00 |
| ATOM | 3971 | HH12 | ARG+ | 572 | −17.802 | −4.096 | 2.471 | 0.00 | 0.00 | 0.407 | 0.00 | 0.00 |
| ATOM | 3972 | NH2 | ARG+ | 572 | −17.693 | −1.567 | 0.360 | 0.00 | 0.00 | −0.746 | 9.00 | −24.67 |
| ATOM | 3973 | HH21 | ARG+ | 572 | −18.480 | −1.015 | 0.730 | 0.00 | 0.00 | 0.407 | 0.00 | 0.00 |
| ATOM | 3974 | HH22 | ARG+ | 572 | −17.161 | −1.226 | −0.453 | 0.00 | 0.00 | 0.407 | 0.00 | 0.00 |
| ATOM | 3975 | N | PRO | 573 | −11.961 | −7.344 | −0.622 | 0.00 | 0.00 | −0.422 | 9.00 | −17.40 |
| ATOM | 3976 | CA | PRO | 573 | −11.291 | −8.579 | −0.339 | 0.00 | 0.00 | 0.158 | 9.40 | 4.00 |
| ATOM | 3977 | HA | PRO | 573 | −11.154 | −8.632 | 0.741 | 0.00 | 0.00 | 0.053 | 0.00 | 0.00 |
| ATOM | 3978 | CD | PRO | 573 | −13.319 | −7.573 | −1.087 | 0.00 | 0.00 | 0.105 | 12.77 | 4.00 |
| ATOM | 3979 | HD1 | PRO | 573 | −13.317 | −7.311 | −2.145 | 0.00 | 0.00 | 0.053 | 0.00 | 0.00 |
| ATOM | 3980 | HD2 | PRO | 573 | −13.951 | −6.917 | −0.488 | 0.00 | 0.00 | 0.053 | 0.00 | 0.00 |
| ATOM | 3981 | C | PRO | 573 | −9.985 | −8.548 | −1.058 | 0.00 | 0.00 | 0.396 | 9.82 | 4.00 |
| ATOM | 3982 | O | PRO | 573 | −9.935 | −8.034 | −2.174 | 0.00 | 0.00 | −0.396 | 8.17 | −17.40 |
| ATOM | 3983 | CB | PRO | 573 | −12.201 | −9.700 | −0.834 | 0.00 | 0.00 | −0.106 | 12.77 | 4.00 |
| ATOM | 3984 | HB1 | PRO | 573 | −12.156 | −10.561 | −0.167 | 0.00 | 0.00 | 0.053 | 0.00 | 0.00 |
| ATOM | 3985 | HB2 | PRO | 573 | −11.906 | −10.030 | −1.830 | 0.00 | 0.00 | 0.053 | 0.00 | 0.00 |
| ATOM | 3986 | CG | PRO | 573 | −13.598 | −9.062 | −0.840 | 0.00 | 0.00 | −0.106 | 12.77 | 4.00 |
| ATOM | 3987 | HG1 | PRO | 573 | −14.104 | −9.223 | 0.112 | 0.00 | 0.00 | 0.053 | 0.00 | 0.00 |
| ATOM | 3988 | HG2 | PRO | 573 | −14.218 | −9.490 | −1.627 | 0.00 | 0.00 | 0.053 | 0.00 | 0.00 |
| ATOM | 3989 | N | HIS | 574 | −8.918 | −9.088 | −0.446 | 0.00 | 0.00 | −0.650 | 9.00 | −17.40 |
| ATOM | 3990 | HN | HIS | 574 | −9.011 | −9.464 | 0.508 | 0.00 | 0.00 | 0.439 | 0.00 | 0.00 |
| ATOM | 3991 | CA | HIS | 574 | −7.654 | −9.144 | −1.112 | 0.00 | 0.00 | 0.158 | 9.40 | 4.00 |
| ATOM | 3992 | HA | HIS | 574 | −7.815 | −9.682 | −2.046 | 0.00 | 0.00 | 0.053 | 0.00 | 0.00 |
| ATOM | 3993 | C | HIS | 574 | −6.721 | −9.869 | −0.202 | 0.00 | 0.00 | 0.396 | 9.82 | 4.00 |
| ATOM | 3994 | O | HIS | 574 | −7.129 | −10.385 | 0.837 | 0.00 | 0.00 | −0.396 | 8.17 | −17.40 |
| ATOM | 3995 | CB | HIS | 574 | −7.059 | −7.769 | −1.467 | 0.00 | 0.00 | −0.106 | 12.77 | 4.00 |
| ATOM | 3996 | HB1 | HIS | 574 | −7.694 | −7.307 | −2.223 | 0.00 | 0.00 | 0.053 | 0.00 | 0.00 |
| ATOM | 3997 | HB2 | HIS | 574 | −6.051 | −7.922 | −1.852 | 0.00 | 0.00 | 0.053 | 0.00 | 0.00 |
| ATOM | 3998 | CG | HIS | 574 | −6.964 | −6.819 | −0.312 | 0.00 | 0.00 | 0.241 | 7.26 | 0.60 |
| ATOM | 3999 | ND1 | HIS | 574 | −8.030 | −6.101 | 0.183 | 0.00 | 0.00 | −0.481 | 9.25 | −17.40 |
| ATOM | 4000 | CD2 | HIS | 574 | −5.890 | −6.457 | 0.441 | 0.00 | 0.00 | −0.177 | 10.80 | 0.60 |
| ATOM | 4001 | HD2 | HIS | 574 | −4.880 | −6.845 | 0.309 | 0.00 | 0.00 | 0.127 | 0.00 | 0.00 |
| ATOM | 4002 | CE1 | HIS | 574 | −7.552 | −5.346 | 1.204 | 0.00 | 0.00 | 0.064 | 10.80 | 0.60 |
| ATOM | 4003 | HE1 | HIS | 574 | −8.165 | −4.667 | 1.796 | 0.00 | 0.00 | 0.127 | 0.00 | 0.00 |
| ATOM | 4004 | NE2 | HIS | 574 | −6.258 | −5.528 | 1.398 | 0.00 | 0.00 | −0.292 | 9.25 | −17.40 |
| ATOM | 4005 | HE2 | HIS | 574 | −5.657 | −5.079 | 2.104 | 0.00 | 0.00 | 0.393 | 0.00 | 0.00 |
| ATOM | 4006 | N | VAL | 575 | −5.435 | −9.962 | −0.590 | 0.00 | 0.00 | −0.650 | 9.00 | −17.40 |
| ATOM | 4007 | HN | VAL | 575 | −5.128 | −9.541 | −1.479 | 0.00 | 0.00 | 0.439 | 0.00 | 0.00 |
| ATOM | 4008 | CA | VAL | 575 | −4.500 | −10.651 | 0.245 | 0.00 | 0.00 | 0.158 | 9.40 | 4.00 |
| ATOM | 4009 | HA | VAL | 575 | −4.879 | −11.659 | 0.413 | 0.00 | 0.00 | 0.053 | 0.00 | 0.00 |
| ATOM | 4010 | C | VAL | 575 | −4.391 | −9.900 | 1.531 | 0.00 | 0.00 | 0.396 | 9.82 | 4.00 |
| ATOM | 4011 | O | VAL | 575 | −4.487 | −10.475 | 2.614 | 0.00 | 0.00 | −0.396 | 8.17 | −17.40 |
| ATOM | 4012 | CB | VAL | 575 | −3.126 | −10.715 | −0.352 | 0.00 | 0.00 | −0.053 | 9.40 | 4.00 |
| ATOM | 4013 | HB | VAL | 575 | −2.785 | −9.698 | −0.546 | 0.00 | 0.00 | 0.053 | 0.00 | 0.00 |
| ATOM | 4014 | CG1 | VAL | 575 | −2.190 | −11.413 | 0.650 | 0.00 | 0.00 | −0.159 | 16.15 | 4.00 |
| ATOM | 4015 | HG11 | VAL | 575 | −1.185 | −11.468 | 0.231 | 0.00 | 0.00 | 0.053 | 0.00 | 0.00 |
| ATOM | 4016 | HG12 | VAL | 575 | −2.165 | −10.846 | 1.580 | 0.00 | 0.00 | 0.053 | 0.00 | 0.00 |
| ATOM | 4017 | HG13 | VAL | 575 | −2.556 | −12.420 | 0.849 | 0.00 | 0.00 | 0.053 | 0.00 | 0.00 |
| ATOM | 4018 | CG2 | VAL | 575 | −3.217 | −11.421 | −1.715 | 0.00 | 0.00 | −0.159 | 16.15 | 4.00 |
| ATOM | 4019 | HG21 | VAL | 575 | −2.225 | −11.476 | −2.163 | 0.00 | 0.00 | 0.053 | 0.00 | 0.00 |
| ATOM | 4020 | HG22 | VAL | 575 | −3.609 | −12.429 | −1.577 | 0.00 | 0.00 | 0.053 | 0.00 | 0.00 |
| ATOM | 4021 | HG23 | VAL | 575 | −3.881 | −10.860 | −2.372 | 0.00 | 0.00 | 0.053 | 0.00 | 0.00 |
| ATOM | 4022 | N | TRP | 576 | −4.225 | −8.570 | 1.437 | 0.00 | 0.00 | −0.650 | 9.00 | −17.40 |
| ATOM | 4023 | HN | TRP | 576 | −4.213 | −8.117 | 0.512 | 0.00 | 0.00 | 0.439 | 0.00 | 0.00 |
| ATOM | 4024 | CA | TRP | 576 | −4.065 | −7.781 | 2.621 | 0.00 | 0.00 | 0.158 | 9.40 | 4.00 |
| ATOM | 4025 | HA | TRP | 576 | −3.401 | −8.329 | 3.289 | 0.00 | 0.00 | 0.053 | 0.00 | 0.00 |
| ATOM | 4026 | C | TRP | 576 | −5.422 | −7.601 | 3.223 | 0.00 | 0.00 | 0.396 | 9.82 | 4.00 |
| ATOM | 4027 | O | TRP | 576 | −6.425 | −8.044 | 2.667 | 0.00 | 0.00 | −0.396 | 8.17 | −17.40 |
| ATOM | 4028 | CB | TRP | 576 | −3.467 | −6.389 | 2.347 | 0.00 | 0.00 | −0.106 | 12.77 | 4.00 |
| ATOM | 4029 | HB1 | TRP | 576 | −4.222 | −5.783 | 1.847 | 0.00 | 0.00 | 0.053 | 0.00 | 0.00 |
| ATOM | 4030 | HB2 | TRP | 576 | −2.591 | −6.510 | 1.710 | 0.00 | 0.00 | 0.053 | 0.00 | 0.00 |
| ATOM | 4031 | CG | TRP | 576 | −3.034 | −5.654 | 3.590 | 0.00 | 0.00 | 0.000 | 7.26 | 0.60 |
| ATOM | 4032 | CD1 | TRP | 576 | −3.706 | −4.757 | 4.366 | 0.00 | 0.00 | −0.177 | 10.80 | 0.60 |
| ATOM | 4033 | HD1 | TRP | 576 | −4.724 | −4.411 | 4.185 | 0.00 | 0.00 | 0.127 | 0.00 | 0.00 |
| ATOM | 4034 | CD2 | TRP | 576 | −1.738 | −5.817 | 4.184 | 0.00 | 0.00 | 0.000 | 6.80 | 0.60 |
| ATOM | 4035 | NE1 | TRP | 576 | −2.909 | −4.353 | 5.412 | 0.00 | 0.00 | −0.292 | 9.00 | −17.40 |
| ATOM | 4036 | HE1 | TRP | 576 | −3.175 | −3.682 | 6.147 | 0.00 | 0.00 | 0.393 | 0.00 | 0.00 |
| ATOM | 4037 | CE2 | TRP | 576 | −1.695 | −4.997 | 5.312 | 0.00 | 0.00 | −0.050 | 6.80 | 0.60 |
| ATOM | 4038 | CE3 | TRP | 576 | −0.672 | −6.589 | 3.822 | 0.00 | 0.00 | −0.127 | 10.80 | 0.60 |
| ATOM | 4039 | HE3 | TRP | 576 | −0.710 | −7.231 | 2.942 | 0.00 | 0.00 | 0.127 | 0.00 | 0.00 |
| ATOM | 4040 | CZ2 | TRP | 576 | −0.579 | −4.937 | 6.097 | 0.00 | 0.00 | −0.127 | 10.80 | 0.60 |
| ATOM | 4041 | HZ2 | TRP | 576 | −0.541 | −4.299 | 6.980 | 0.00 | 0.00 | 0.127 | 0.00 | 0.00 |
| ATOM | 4042 | CZ3 | TRP | 576 | 0.453 | −6.524 | 4.614 | 0.00 | 0.00 | −0.127 | 10.80 | 0.60 |
| ATOM | 4043 | HZ3 | TRP | 576 | 1.325 | −7.124 | 4.354 | 0.00 | 0.00 | 0.127 | 0.00 | 0.00 |
| ATOM | 4044 | CH2 | TRP | 576 | 0.497 | −5.714 | 5.730 | 0.00 | 0.00 | −0.127 | 10.80 | 0.60 |
| ATOM | 4045 | HH2 | TRP | 576 | 1.404 | −5.689 | 6.335 | 0.00 | 0.00 | 0.127 | 0.00 | 0.00 |
| ATOM | 4046 | N | THR | 577 | −5.478 | −6.960 | 4.406 | 0.00 | 0.00 | −0.650 | 9.00 | −17.40 |
| ATOM | 4047 | HN | THR | 577 | −4.609 | −6.605 | 4.830 | 0.00 | 0.00 | 0.439 | 0.00 | 0.00 |
| ATOM | 4048 | CA | THR | 577 | −6.724 | −6.763 | 5.087 | 0.00 | 0.00 | 0.158 | 9.40 | 4.00 |
| ATOM | 4049 | HA | THR | 577 | −7.361 | −7.638 | 4.956 | 0.00 | 0.00 | 0.053 | 0.00 | 0.00 |
| ATOM | 4050 | C | THR | 577 | −7.427 | −5.557 | 4.530 | 0.00 | 0.00 | 0.396 | 9.82 | 4.00 |
| ATOM | 4051 | O | THR | 577 | −6.826 | −4.710 | 3.870 | 0.00 | 0.00 | −0.396 | 8.17 | −17.40 |
| ATOM | 4052 | CB | THR | 577 | −6.570 | −6.553 | 6.565 | 0.00 | 0.00 | 0.060 | 9.40 | 4.00 |
| ATOM | 4053 | HB | THR | 577 | −5.976 | −7.370 | 6.975 | 0.00 | 0.00 | 0.053 | 0.00 | 0.00 |
| ATOM | 4054 | OG1 | THR | 577 | −7.841 | −6.556 | 7.197 | 0.00 | 0.00 | −0.537 | 11.04 | −17.40 |
| ATOM | 4055 | HG1 | THR | 577 | −8.151 | −5.586 | 7.351 | 0.00 | 0.00 | 0.424 | 0.00 | 0.00 |
| ATOM | 4056 | CG2 | THR | 577 | −5.859 | −5.209 | 6.803 | 0.00 | 0.00 | −0.159 | 16.15 | 4.00 |
| ATOM | 4057 | HG21 | THR | 577 | −5.741 | −5.044 | 7.874 | 0.00 | 0.00 | 0.053 | 0.00 | 0.00 |
| ATOM | 4058 | HG22 | THR | 577 | −4.878 | −5.227 | 6.328 | 0.00 | 0.00 | 0.053 | 0.00 | 0.00 |
| ATOM | 4059 | HG23 | THR | 577 | −6.454 | −4.402 | 6.375 | 0.00 | 0.00 | 0.053 | 0.00 | 0.00 |
| ATOM | 4060 | N | VAL | 578 | −8.752 | −5.496 | 4.776 | 0.00 | 0.00 | −0.650 | 9.00 | −17.40 |
| ATOM | 4061 | HN | VAL | 578 | −9.146 | −6.262 | 5.340 | 0.00 | 0.00 | 0.439 | 0.00 | 0.00 |
| ATOM | 4062 | CA | VAL | 578 | −9.680 | −4.483 | 4.341 | 0.00 | 0.00 | 0.158 | 9.40 | 4.00 |
| ATOM | 4063 | HA | VAL | 578 | −9.565 | −4.236 | 3.286 | 0.00 | 0.00 | 0.053 | 0.00 | 0.00 |
| ATOM | 4064 | C | VAL | 578 | −9.519 | −3.195 | 5.093 | 0.00 | 0.00 | 0.396 | 9.82 | 4.00 |
| ATOM | 4065 | O | VAL | 578 | −9.875 | −2.130 | 4.588 | 0.00 | 0.00 | −0.396 | 8.17 | −17.40 |
| ATOM | 4066 | CB | VAL | 578 | −11.111 | −4.910 | 4.503 | 0.00 | 0.00 | −0.053 | 9.40 | 4.00 |
| ATOM | 4067 | HB | VAL | 578 | −11.293 | −5.133 | 5.554 | 0.00 | 0.00 | 0.053 | 0.00 | 0.00 |
| ATOM | 4068 | CG1 | VAL | 578 | −12.025 | −3.762 | 4.037 | 0.00 | 0.00 | −0.159 | 16.15 | 4.00 |
| ATOM | 4069 | HG11 | VAL | 578 | −13.068 | −4.059 | 4.149 | 0.00 | 0.00 | 0.053 | 0.00 | 0.00 |
| ATOM | 4070 | HG12 | VAL | 578 | −11.833 | −2.876 | 4.642 | 0.00 | 0.00 | 0.053 | 0.00 | 0.00 |
| ATOM | 4071 | HG13 | VAL | 578 | −11.822 | −3.538 | 2.990 | 0.00 | 0.00 | 0.053 | 0.00 | 0.00 |
| ATOM | 4072 | CG2 | VAL | 578 | −11.316 | −6.226 | 3.730 | 0.00 | 0.00 | −0.159 | 16.15 | 4.00 |
| ATOM | 4073 | HG21 | VAL | 578 | −12.351 | −6.553 | 3.836 | 0.00 | 0.00 | 0.053 | 0.00 | 0.00 |
| ATOM | 4074 | HG22 | VAL | 578 | −11.092 | −6.067 | 2.675 | 0.00 | 0.00 | 0.053 | 0.00 | 0.00 |
| ATOM | 4075 | HG23 | VAL | 578 | −10.651 | −6.991 | 4.131 | 0.00 | 0.00 | 0.053 | 0.00 | 0.00 |
| ATOM | 4076 | N | ASP− | 579 | −9.013 | −3.248 | 6.336 | 0.00 | 0.00 | −0.650 | 9.00 | −17.40 |
| ATOM | 4077 | HN | ASP− | 579 | −8.654 | −4.137 | 6.714 | 0.00 | 0.00 | 0.439 | 0.00 | 0.00 |
| ATOM | 4078 | CA | ASP− | 579 | −8.977 | −2.054 | 7.134 | 0.00 | 0.00 | 0.158 | 9.40 | 4.00 |
| ATOM | 4079 | HA | ASP− | 579 | −9.990 | −1.690 | 7.308 | 0.00 | 0.00 | 0.053 | 0.00 | 0.00 |
| ATOM | 4080 | C | ASP− | 579 | −8.193 | −0.989 | 6.430 | 0.00 | 0.00 | 0.396 | 9.82 | 4.00 |
| ATOM | 4081 | O | ASP− | 579 | −8.634 | 0.156 | 6.351 | 0.00 | 0.00 | −0.396 | 8.17 | −17.40 |
| ATOM | 4082 | CB | ASP− | 579 | −8.321 | −2.272 | 8.508 | 0.00 | 0.00 | −0.336 | 12.77 | 4.00 |
| ATOM | 4083 | HB1 | ASP− | 579 | −7.252 | −2.424 | 8.357 | 0.00 | 0.00 | 0.053 | 0.00 | 0.00 |
| ATOM | 4084 | HB2 | ASP− | 579 | −8.773 | −3.152 | 8.967 | 0.00 | 0.00 | 0.053 | 0.00 | 0.00 |
| ATOM | 4085 | CG | ASP− | 579 | −8.572 | −1.033 | 9.360 | 0.00 | 0.00 | 0.297 | 9.82 | 4.00 |
| ATOM | 4086 | OD1 | ASP− | 579 | −9.160 | −0.055 | 8.826 | 0.00 | 0.00 | −0.534 | 8.17 | −18.95 |
| ATOM | 4087 | OD2 | ASP− | 579 | −8.178 | −1.048 | 10.557 | 0.00 | 0.00 | −0.534 | 8.17 | −18.95 |
| ATOM | 4088 | N | LEU | 580 | −7.025 | −1.341 | 5.867 | 0.00 | 0.00 | −0.650 | 9.00 | −17.40 |
| ATOM | 4089 | HN | LEU | 580 | −6.724 | −2.326 | 5.882 | 0.00 | 0.00 | 0.439 | 0.00 | 0.00 |
| ATOM | 4090 | CA | LEU | 580 | −6.196 | −0.350 | 5.246 | 0.00 | 0.00 | 0.158 | 9.40 | 4.00 |
| ATOM | 4091 | HA | LEU | 580 | −5.920 | 0.408 | 5.980 | 0.00 | 0.00 | 0.053 | 0.00 | 0.00 |
| ATOM | 4092 | C | LEU | 580 | −6.944 | 0.297 | 4.121 | 0.00 | 0.00 | 0.396 | 9.82 | 4.00 |
| ATOM | 4093 | O | LEU | 580 | −6.917 | 1.519 | 3.977 | 0.00 | 0.00 | −0.396 | 8.17 | −17.40 |
| ATOM | 4094 | CB | LEU | 580 | −4.891 | −0.941 | 4.683 | 0.00 | 0.00 | −0.106 | 12.77 | 4.00 |
| ATOM | 4095 | HB1 | LEU | 580 | −4.315 | −0.230 | 4.091 | 0.00 | 0.00 | 0.053 | 0.00 | 0.00 |
| ATOM | 4096 | HB2 | LEU | 580 | −5.052 | −1.797 | 4.028 | 0.00 | 0.00 | 0.053 | 0.00 | 0.00 |
| ATOM | 4097 | CG | LEU | 580 | −3.925 | −1.439 | 5.776 | 0.00 | 0.00 | −0.053 | 9.40 | 4.00 |
| ATOM | 4098 | HG | LEU | 580 | −3.081 | −1.976 | 5.343 | 0.00 | 0.00 | 0.053 | 0.00 | 0.00 |
| ATOM | 4099 | CD1 | LEU | 580 | −3.360 | −0.268 | 6.597 | 0.00 | 0.00 | −0.159 | 16.15 | 4.00 |
| ATOM | 4100 | HD11 | LEU | 580 | −2.682 | −0.652 | 7.360 | 0.00 | 0.00 | 0.053 | 0.00 | 0.00 |
| ATOM | 4101 | HD12 | LEU | 580 | −2.818 | 0.410 | 5.938 | 0.00 | 0.00 | 0.053 | 0.00 | 0.00 |
| ATOM | 4102 | HD13 | LEU | 580 | −4.179 | 0.269 | 7.076 | 0.00 | 0.00 | 0.053 | 0.00 | 0.00 |
| ATOM | 4103 | CD2 | LEU | 580 | −4.580 | −2.512 | 6.662 | 0.00 | 0.00 | −0.159 | 16.15 | 4.00 |
| ATOM | 4104 | HD21 | LEU | 580 | −3.870 | −2.840 | 7.421 | 0.00 | 0.00 | 0.053 | 0.00 | 0.00 |
| ATOM | 4105 | HD22 | LEU | 580 | −5.463 | −2.095 | 7.147 | 0.00 | 0.00 | 0.053 | 0.00 | 0.00 |
| ATOM | 4106 | HD23 | LEU | 580 | −4.872 | −3.363 | 6.047 | 0.00 | 0.00 | 0.053 | 0.00 | 0.00 |
| ATOM | 4107 | N | ASN | 581 | −7.656 | −0.492 | 3.295 | 0.00 | 0.00 | −0.650 | 9.00 | −17.40 |
| ATOM | 4108 | HN | ASN | 581 | −7.701 | −1.512 | 3.435 | 0.00 | 0.00 | 0.439 | 0.00 | 0.00 |
| ATOM | 4109 | CA | ASN | 581 | −8.348 | 0.143 | 2.215 | 0.00 | 0.00 | 0.158 | 9.40 | 4.00 |
| ATOM | 4110 | HA | ASN | 581 | −7.614 | 0.690 | 1.623 | 0.00 | 0.00 | 0.053 | 0.00 | 0.00 |
| ATOM | 4111 | C | ASN | 581 | −9.358 | 1.067 | 2.801 | 0.00 | 0.00 | 0.396 | 9.82 | 4.00 |
| ATOM | 4112 | O | ASN | 581 | −9.577 | 2.164 | 2.290 | 0.00 | 0.00 | −0.396 | 8.17 | −17.40 |
| ATOM | 4113 | CB | ASN | 581 | −9.043 | −0.820 | 1.223 | 0.00 | 0.00 | −0.106 | 12.77 | 4.00 |
| ATOM | 4114 | HB1 | ASN | 581 | −8.340 | −1.519 | 0.769 | 0.00 | 0.00 | 0.053 | 0.00 | 0.00 |
| ATOM | 4115 | HB2 | ASN | 581 | −9.527 | −0.287 | 0.405 | 0.00 | 0.00 | 0.053 | 0.00 | 0.00 |
| ATOM | 4116 | CG | ASN | 581 | −10.120 | −1.658 | 1.898 | 0.00 | 0.00 | 0.396 | 9.82 | 4.00 |
| ATOM | 4117 | OD1 | ASN | 581 | −9.918 | −2.843 | 2.153 | 0.00 | 0.00 | −0.396 | 8.17 | −17.40 |
| ATOM | 4118 | ND2 | ASN | 581 | −11.299 | −1.042 | 2.175 | 0.00 | 0.00 | −0.879 | 13.25 | −17.40 |
| ATOM | 4119 | HD21 | ASN | 581 | −11.426 | −0.046 | 1.945 | 0.00 | 0.00 | 0.439 | 0.00 | 0.00 |
| ATOM | 4120 | HD22 | ASN | 581 | −12.065 | −1.571 | 2.615 | 0.00 | 0.00 | 0.439 | 0.00 | 0.00 |
| ATOM | 4121 | N | ASN | 582 | −9.999 | 0.644 | 3.904 | 0.00 | 0.00 | −0.650 | 9.00 | −17.40 |
| ATOM | 4122 | HN | ASN | 582 | −9.776 | −0.274 | 4.315 | 0.00 | 0.00 | 0.439 | 0.00 | 0.00 |
| ATOM | 4123 | CA | ASN | 582 | −10.992 | 1.480 | 4.500 | 0.00 | 0.00 | 0.158 | 9.40 | 4.00 |
| ATOM | 4124 | HA | ASN | 582 | −11.757 | 1.737 | 3.768 | 0.00 | 0.00 | 0.053 | 0.00 | 0.00 |
| ATOM | 4125 | C | ASN | 582 | −10.368 | 2.745 | 5.008 | 0.00 | 0.00 | 0.396 | 9.82 | 4.00 |
| ATOM | 4126 | O | ASN | 582 | −10.850 | 3.834 | 4.701 | 0.00 | 0.00 | −0.396 | 8.17 | −17.40 |
| ATOM | 4127 | CB | ASN | 582 | −11.689 | 0.825 | 5.707 | 0.00 | 0.00 | −0.106 | 12.77 | 4.00 |
| ATOM | 4128 | HB1 | ASN | 582 | −12.291 | 1.578 | 6.215 | 0.00 | 0.00 | 0.053 | 0.00 | 0.00 |
| ATOM | 4129 | HB2 | ASN | 582 | −10.928 | 0.434 | 6.382 | 0.00 | 0.00 | 0.053 | 0.00 | 0.00 |
| ATOM | 4130 | CG | ASN | 582 | −12.576 | −0.306 | 5.206 | 0.00 | 0.00 | 0.396 | 9.82 | 4.00 |
| ATOM | 4131 | OD1 | ASN | 582 | −13.022 | −0.296 | 4.060 | 0.00 | 0.00 | −0.396 | 8.17 | −17.40 |
| ATOM | 4132 | ND2 | ASN | 582 | −12.851 | −1.303 | 6.088 | 0.00 | 0.00 | −0.879 | 13.25 | −17.40 |
| ATOM | 4133 | HD21 | ASN | 582 | −12.457 | −1.273 | 7.039 | 0.00 | 0.00 | 0.439 | 0.00 | 0.00 |
| ATOM | 4134 | HD22 | ASN | 582 | −13.454 | −2.089 | 5.805 | 0.00 | 0.00 | 0.439 | 0.00 | 0.00 |

TABLE 24
N-acetylglucosyaminyltransferases

| Enzyme | Donor | Acceptor |
|---|---|---|
| α-1,3-mannosyl-glycoprotein | UDP-D-GlcNAc | α-D-mannosyl-1,3 (R1)- |
| β-1,2 N-acetylglucosaminyl | | β-D-mannosyl-R2 |
| transferase | | |
| (GnT I) (EC 2.4.1.101) | | |
| α-1,6-mannosyl-glycoprotein-β | UDP-D-GlcNAc | α-D-mannosyl-1,6 (N-acetyl- |
| 1,2 N-acetylglucosaminyl transferase | | β-D glucosaminyl-1,2-α-D- |
| (GAT) (GnT II) (EC 2.4.1.143) | | (mannosyl-1,3-)-β-D- |
| | | mannosyl-R |
| α-1,3(6)-mannosylglycoprotein | UDP-D-GlcNAc | N-acetyl-β-D-glucosaminyl- |
| β 1,6-N-acetyl-glucosaminyl transferase | | 1,2 alpha-D-mannosyl- |
| (EC 2.4.1.155) (GnT V) | | 1,3(6)-(N-acetyl-β-D- |
| | | glucosaminyl-1,2-α-D-mannosyl- |
| | | 1,6(3)-βD-mannosyl-1,4-N-acetyl-β-D- |
| | | glucosaminyl-R |
| β-1,3-galactosyl-O-glycosyl | UDP-D-GlcNAc | β-D-galactosyl-1,3 N- |
| glycoprotein-β1,6-N acetylglucosaminyl | | acetyl-D-galactosaminyl-R |
| transferase (EC 2.4.1.102) | | |
| (β1,6 (O-linked, core 2)) | | |
| β-1,4-mannosyl-glycoprotein | UDP-D-GlcNAc | N-acetyl-β-D-glucosaminyl-1,2 |
| β-1,4-N-acetylglucosaminyl transferase | | alpha-D-mannosyl-1,3-(N-acetyl- |
| (GnT III) (EC 2.4.1.144) | | β-D-glucosaminyl-1,2-α-D- |
| | | mannosyl-1,6)-β-D-mannosyl-1,4- |
| | | N-acetyl-β-D-glucosaminyl-R |

TABLE 25
^{a}Ab initio Calculated Geometrical Parameters of the Points observed on PES's described
on FIGS. 1 (a) through 3 (b) at the HF/6-31G* and DFT/B3LYP/6-31G* levels

| | | | | | | | | | | | | | Torsional | |
|---|---|---|---|---|---|---|---|---|---|---|---|---|---|---|
| | Bond Lengths | | | | | | | | | | Bond Angles | | Angles | |
| | Cl-Oa | | Ha-Oa | | HA-O1 | | Cl-O1 | | Cl-O5 | | Cl-O5-C5 | | Cl-O5-C5-C4 | |
| Conformer | HF | B3LYP | HF | B3LYP | HF | B3LYP | HF | B3LYP | HF | B3LYP | HF | B3LYP | HF | B3LYP |
| R | 2.800 | 2.800 | 0.959 | 0.985 | 1.847 | 1.818 | 1.478 | 1.519 | 1.359 | 1.371 | 120.2 | 119.8 | 58.3 | 59.3 |
| INT1 | 1.551 | 1.532 | 0.994 | 1.055 | 1.832 | 1.801 | 2.985 | 2.829 | 1.333 | 1.369 | 125.9 | 124.3 | 9.7 | 46.5 |
| INT2 | 2.800 | 2.800 | 2.026 | 1.981 | 1.848 | 1.848 | 1.492 | 1.529 | 1.368 | 1.381 | 120.1 | 120.5 | 56.4 | 54.2 |
| INT3 | 2.800 | 2.800 | 2.044 | 2.021 | 0.990 | 1.112 | 2.731 | 2.663 | 1.230 | 1.253 | 127.4 | 125.4 | 20.4 | 21.0 |
| INT4 | 2.800 | 2.800 | 0.961 | 0.988 | 0.987 | 1.038 | 3.288 | 3.733 | 1.233 | 1.259 | 125.5 | 122.2 | 42.4 | 48.0 |
| INT5 | 1.502 | 1.653 | 1.000 | 1.032 | 0.988 | 1.035 | 3.306 | 3.467 | 1.350 | 1.330 | 125.1 | 123.6 | 43.4 | 7.5 |
| TS1 | 2.003 | 2.158 | 0.973 | 1.000 | 1.834 | 1.806 | 2.686 | 2.535 | 1.277 | 1.290 | 124.3 | 123.6 | 51.3 | 52.2 |
| TS2 | 1.447 | 1.499 | 1.380 | 1.361 | 1.832 | 1.800 | 3.009 | 3.014 | 1.371 | 1.379 | 123.8 | 124.9 | 47.6 | 27.5 |
| TS3 | 2.800 | 2.800 | 1.599 | 1.638 | 1.848 | 1.848 | 1.489 | 1.524 | 1.364 | 1.378 | 119.6 | 120.0 | 56.5 | 56.6 |
| TS4 | 2.490 | 2.426 | 2.030 | 1.993 | 1.848 | 1.811 | 1.821 | 1.863 | 1.301 | 1.331 | 120.3 | 118.8 | 47.7 | 55.0 |
| TS5 | 1.399 | 1.423 | 2.067 | 2.044 | 1.291 | 1.221 | 3.229 | 3.150 | 1.391 | 1.412 | 122.9 | 121.5 | 47.6 | 48.0 |
| TS6 | 2.800 | 2.800 | 2.035 | 2.013 | 1.280 | 1.228 | 2.454 | 2.668 | 1.251 | 1.289 | 123.6 | 121.3 | 43.6 | 47.2 |
| TS7 | 2.476 | 2.379 | 2.040 | 2.027 | 1.005 | 1.081 | 2.728 | 2.540 | 1.254 | 1.296 | 125.1 | 123.9 | 34.4 | 39.0 |
| TS8 | 1.546 | 1.626 | 0.996 | 1.036 | 1.304 | 1.247 | 2.908 | 2.843 | 1.333 | 1.337 | 125.7 | 123.9 | 13.3 | 5.9 |
| TS9 | 2.800 | 2.800 | 0.961 | 0.987 | 1.239 | 1.169 | 1.846 | 1.950 | 1.282 | 1.286 | 122.1 | 121.9 | 58.0 | 57.3 |
| TS10 | 2.656 | 2.653 | 0.960 | 0.991 | 0.992 | 1.141 | 3.403 | 2.068 | 1.237 | 1.276 | 125.2 | 123.4 | 45.9 | 51.6 |
| TS11 | 1.457 | 1.518 | 1.300 | 1.362 | 1.006 | 1.070 | 3.063 | 2.897 | 1.364 | 1.363 | 124.0 | 123.1 | 52.5 | 20.4 |
| PC1 | 1.397 | 1.424 | 2.067 | 2.044 | 1.833 | 1.802 | 3.206 | 3.260 | 1.394 | 1.413 | 122.2 | 121.0 | 51.5 | 49.4 |
| PC2 | 1.395 | 1.428 | 2.067 | 2.046 | 1.007 | 1.076 | 3.270 | 3.253 | 1.392 | 1.410 | 122.3 | 122.5 | 53.0 | 43.1 |

^{a}Lengths in angstroms, angles in degrees.

TABLE 26
Comparison of the ab initio Relative Energies (kcal/mol)
Calculated by Various Methods for the Points observed
on PES's described on FIGS. 1 (a) through 3 (b).

| Geometry | HF/6-31G* | HF/6-31G* | B3LYP/6-31G* | B3LYP/6-31G* |
|---|---|---|---|---|
| Energy | 6-31G* | 6-31++G** | 6-31G* | 6-31++G** |
| R | 0.00^{a} | 0.00^{b} | 0.00^{c} | 0.00^{d} |
| INT1 | 11.52 | 11.95 | 10.28 | 7.60 |
| INT2 | 35.33 | 34.18 | 32.82 | 30.41 |
| INT3 | 60.10 | 56.65 | 57.86 | 54.36 |
| INT4 | 33.76 | 30.63 | 29.25 | 25.77 |
| INT5 | 30.33 | 28.29 | 19.90 | 17.34 |
| TS1 | 19.32 | 18.38 | 16.06 | 13.35 |
| TS2 | 26.20 | 27.11 | 14.39 | 14.65 |
| TS3 | 50.45 | 50.53 | 37.63 | 36.34 |
| TS4 | 39.17 | 38.55 | 34.94 | 32.20 |
| TS5 | 6.30 | 8.22 | 1.32 | −0.22 |
| TS6 | 81.78 | 77.92 | 66.96 | 59.80 |
| TS7 | 70.84 | 64.89 | 62.43 | 54.68 |
| TS8 | 43.18 | 42.98 | 28.12 | 26.36 |
| TS9 | 54.59 | 53.27 | 39.88 | 37.55 |
| TS10 | 47.68 | 41.42 | 40.96 | 37.64 |
| TS11 | 53.50 | 52.28 | 37.92 | 34.35 |
| PC1 | −26.22 | −23.87 | −21.98 | −22.37 |
| PC2 | −1.35 | −1.81 | 0.98 | −2.99 |

^{a}E = −2225097.02 kcal/mol,
^{b}E = −2225204.97 kcal/mol,
^{c}E = −2235455.59 kcal/mol,
^{d}E = −2235585.22 kcal/mol.

We claim

1. A model or secondary, tertiary, and/or quanternary structure for a ligand binding domain of a glycosyltransferase.

2. A model as claimed in claim 1 wherein the ligand binding domain is a binding domain for a disphosphate group of a sugar nucleotide donor, a nucleotide of a sugar nucleotide donor, a nitrogeneous heterocyclic base of a sugar nucleotide donor, a sugar of a nucleotide of a sugar nucleotide donor, a selected sugar of a sugar nucleotide donor that is transferred to an acceptor, or an acceptor.

3. A model as claimed in claim 1 wherein the ligand binding domain is defined by (a) one or more amino acid residues of a GnTI shown in Table 10; (b) one or more amino acid residues of a GnTV shown in Table 11; (c) one or more amino acid residues of a core 2L/T1 shown in Table 12; and (d) one or more amino acid residues of a core 2b/2M/T2 shown in Table 13.

4. A model as claimed in claim 1 defined by the structural coordinates of one or more of the atomic contacts or atomic interactions as shown in Table 10, Table 11, Table 12, or Table 13.

5. A model as claimed in claim 4 wherein each of the atomic interactions is defined in Table 10, 11, 12, or 13 by an atomic contact (more preferably a specific atom where indicated) on a sugar nucleotide donor or part thereof and an atomic contact (more preferably a specific atom where indicated) on the glycosyltransferase.

6. A model as claimed in claim 1 for a transition state of a glycosyltransferase.

7. A model as claimed in claim 1 wherein the glycosyltransferases is selected from the group consisting of GnT1, GnTV, Core 2L/T1, and Core 2b/M/T2, and the ligand binding domain is defined by selected atomic interactions or contacts in the ligand binding domain, as follows:
(a) one or more of atomic interactions or atomic contacts for GnTI shown in Table 10;
(b) one or more of atomic interactions or atomic contacts for GnTV shown in Table 11;
(c) one or more of atomic interactions or atomic contacts for Core 2L/T1 shown in Table 12; or
(d) one or more of atomic interactions or atomic contacts for Core 2b/T shown in Table 13.

8. A model as claimed in claim 1 wherein the glycosyltransferase is selected from the group consisting of GnT1, GnTV, Core 2L/T1, Core 2b/M/T2, Core 2c, and Core 3 comprising the following atomic structural coordinates:
Table 1—structural coordinates for GnTI:
Table 2—Structural coordinates for GnTV.
Table 3, 4, or 5—Structural coordinates for core 2L or T1
Table 6—Structural coordinates for core 2b/core M/core 2 T2.
Table 7—Structural coordinates for core 2C (human)
Table 8—Structural coordinates for core 3.

9. A model as claimed in claim 2 wherein the ligand binding domain associates with a diphosphate of a sugar nucleotide donor and comprises (a) atomic interaction 7 listed in Table 10 (GnTI Table); (b) at least two of atomic interactions 9, 10, 11, 12, and 13 listed in Table 12 (Core 2L Table); (c) at least two of atomic interactions 11, 12, 13, 14, and 15 listed in Table 13 (Core2b/M); or (d) atomic interaction 8 listed in Table 11 (GNTV Table), each atomic interaction defined therein by a residue (more preferably a specific atom where indicated) on the diphosphate of the sugar nucleotide donor and an amino acid, (more preferably a specific atom where indicated), on the glycosyltransferase.

10. A model as claimed in claim 2 wherein the ligand binding domain associates with a heterocyclic amine base, preferably uracil, of a sugar nucleotide donor and comprises at least two of the following atomic interactions (a) 1, 2, 3, and 4 listed in Table 10 (GnTI Table); (b) 1, 2, 3, 4, and 5 listed in Table 12 (Core 2L Table); (c) 1, 2, 3, and 4 listed in Table 13 (Core2b/M); or (d) 1, 2, 3, and 4, listed in Table 11 (GNTV Table), each atomic interaction defined therein by a residue (more preferably a specific atom where indicated) on the heterocyclic amine base of the sugar nucleotide donor and an amino acid, (more preferably a specific atom where indicated), on the glycosyltransferase.

11. A model as claimed in claim 2 wherein the ligand binding domain associates with a sugar, preferably ribose, of the nucleotide of a sugar nucleotide donor and comprises atomic interactions 5 and 6 listed in Table 10 (GnTI Table); at least two of atomic interactions 6, 7, and 8 listed in Table 12 (Core 2L Table), or atomic interaction 5 listed in Table 11 (GNTV Table), each atomic interaction defined therein by a residue (more preferably a specific atom where indicated) on the sugar of the nucleotide of the sugar nucleotide donor and an amino acid, (more preferably a specific atom where indicated), on the glycosyltransferase.

12. A model as claimed in claim 2 wherein the ligand binding domain associates with the sugar, preferably GlcNAc, of a sugar nucleotide donor and comprises at least two of atomic interactions 8, 9, 10, 11, or 12 listed in Table 10 (GnTI Table); at least two of atomic interactions 14, 15, 16, 17, and 18 listed in Table 12 (Core 2L Table), atomic interactions 16 and/or 17 listed in Table 13 (Core2b/M), or at least two of atomic interactions 9, 10, 11, 12 and 13 listed in Table 11 (GNTV Table), each atomic interaction defined therein by a residue (more preferably a specific atom where indicated) on the sugar of the sugar nucleotide donor and an amino acid, (more preferably a specific atom where indicated), on the glycosyltransferase.

13. A model as claimed in claim 2 wherein the ligand binding domain associates with UDP and is characterized by (a) a hydrogen bond between an Asp side chain of the glycosyltransferase with position 3 of the uracil ring of UDP; (b) a stacking interaction between either a disulfide or an aromatic group (Phe or Tyr) of the glycosyltransferase and the uracil ring of the UDP; (c) a stacking interaction between either an Ile or a Thr of the glycosyltransferase and the ribose ring of the UDP; and (d) metal mediated charge interactions between a well-conserved Asp/Glu of the glycosyltransferase and a pyrophosphate oxygen of the UDP.

14. A model as claimed in claim 2 wherein the ligand binding domain associates with a nucleotide, preferably UDP, of a sugar nucleotide donor comprising at least two of (a) atomic interactions 1, 2, 3, 4, 5, 6, and/or 7 listed in Table 10 (GnTI Table); (b) atomic interactions 1, 2, 3, 4, 5, 6, 7, 8, 9, 10, 11, 12, and 13 listed in Table 12 (Core 2L Table); (c) atomic interactions 1, 2, 3, 4, 5, 6, 7, 8, 9, 10, 11, 12, 13, 14, and 15 listed in Table 13 (Core2b/M); or (d) atomic interactions 1, 2, 3, 4, 5, 6, 7, and 8 listed in Table 11 (GNTV Table), each atomic interaction defined therein by a residue (more preferably a specific atom where indicated) on the nucleotide of the sugar nucleotide donor and an amino acid, (more preferably a specific atom where indicated), on the glycosyltransferase.

15. A model as claimed in claim 2 wherein the ligand binding domain associates with a sugar nucleotide donor, preferably UDP-GlcNAc comprising at least two of (a) atomic interactions 1, 2,3, 4, 5, 6, 7, 8, 9, 10, 11, and 12 listed in Table 10 (GnTI Table); (b) atomic interactions 1, 2, 3, 4, 5, 6, 7, 8, 9, 10, 11, 12, 13, 14, 15, 16, 17, and 18 listed in Table 12 (Core 2L Table); (c) atomic interactions 1, 2, 3, 4, 5, 6, 9, 10, 11, 12, 13, 14, 15, 16, and 17 listed in Table 13 (Core2b/M), or (d) atomic interactions 1, 2, 3, 4, 5, 6, 7, 8, 9, 10, 11, 12, and 13 listed in Table 13 (GNTV Table), each atomic interaction defined therein by a residue (more preferably a specific atom where indicated) on the sugar nucleotide donor and an amino acid, (more preferably a specific atom where indicated), on the glycosyltransferase.

16. A model as claimed in claim 2 wherein the ligand binding domain is a loop structure that associates with a pyrophosphate of a sugar nucleotide donor comprising the structural coordinates for the loop structure of GnTI listed in Table 21; Core 2L listed in Table 22; or GnTV listed in Table 24.

17. A model as claimed in claim 6 wherein the ligand binding domain is a GlcNAc transition state ligand binding domain of a glycosyltransferase comprising a hydrophobic pocket that is 1.9 to 3.5 Å, preferably 2.2 to 3.0 Å, from the pyrophosphate binding cavity for the glycosyltransferase.

18. A model as claimed in claim 17 wherein the ligand binding domain is further characterized as follows:
amino acid residues in the domain that associate with C2 and C4 positions of the sugar comprise the structural coordinates of Leu-331, and Leu 269 in Table 1, or the structural coordinates of Leu -116 and Val-81 of Table 3, 4, or 5.

19. A model as claimed in claim 18 wherein the ligand binding domain comprises atomic interactions 14 to 18 in Table 12, atomic interactions 9 to 12 of Table 10, or the particular structural coordinates for the atoms of the atomic contacts of the atomic interactions as set out in Tables 1, 3, 4, or 5.

20. A model according to any preceding claims in association with a ligand or substrate.

21. A computer readable medium having stored thereon a model according to any preceding claim.

22. A computerized representation of a model according to any of the preceding claims.

23. A method of screening for a ligand capable of binding a ligand binding domain of a glycosyltransferase comprising the use of a model according to any preceding claim.

24. A ligand identified by a method according to claim 23.

25. A ligand according to claim 24 that is capable of associating with one or more atomic contacts of a glycosyltransferase as shown in Table 10, 11, 12, or 13.

26. A method of identifying a modulator of a glycosyltransferase or a ligand binding domain thereof comprising the step of using the structural coordinates of a glycosyltransferase or a ligand binding domain thereof as shown in Table 1, 2, 3, 4, 5, 6, 7, or 8, or a model according to any preceding claim to computationally evaluate a test compound for its ability to associate with the glycosyltransferase or binding domain or binding site thereof.

27. A method for identifying a potential modulator of a glycosyltransferase by determining binding interactions between a test compound and atomic contacts of a model of a ligand binding domain of a glycosyltransferase as claimed in any preceding claim comprising:
(a) generating the atomic contacts on a computer screen;
(b) generating test compounds with their spatial structure on the computer screen; and
(c) determining whether the compounds associate or interact with the atomic contacts defining the glycosyltransferase;
(d) identifying test compounds that are potential modulators by their ability to enter into a selected number of atomic contacts.

28. A method for identifying a potential modulator of a glycosyltransferase function by docking a computer representation of a test compound with a computer representation of a model of a glycosyltransferase or a ligand binding domain as claimed in any preceding claim.

29. A method for the design of ligands for glycosyltransferase based on a secondary, tertiary, or quanternary structure or model of a sugar nucleotide donor or part thereof comprising using the structural coordinates shown in Table 14, 15, or 16.

30. A method as claimed in claim 29 comprising (a) generating a computer representation of a sugar nucleotide donor, or part thereof, defined by the structural coordinates shown in Table 14, 15, or 16; (b) searching for molecules in a data base that are similar to the defined sugar nucleotide donor, or part thereof, using a searching computer program, or replacing portions of the compound with similar chemical structures from a database using a compound building computer program.

31. A method as claimed in claim 30 comprising one or more of the following additional steps:
(a) testing whether the ligand is a modulator of the activity of a glycosyltransferase in cellular assays and animal model assays;
(b) modifying the ligand;
(c) optionally rerunning steps (a) or (b); and
(d) preparing a pharmaceutical composition comprising the modulator.

32. A method for designing potential modulators that are inhibitors of a glycosyltransferase, preferably GnT I, GnT V, and/or Core 2L GnT, comprising the step of using one or more (preferably all) of the structural coordinates of uracil, uridine, ribose, pyrophosphate, or UDP of Tables 14, 15 or 16, as follows:
Table 14 for GnTI Ground State
Table 15 for GntV
Table 16 for core 2L
to generate a compound for associating with a ligand binding domain of a glycosyltransferase that associates with uracil, uridine, ribose, pyrophosphate, or UDP.

33. A method for generating a compound for associating with the active site of a glycosyltransferase comprising the following steps: (a) generating a computer representation of uracil, uridine, or UDP defined by structural coordinates of Tables 14, 15 or 16; (b) searching for molecules in a data base that are structurally or chemically similar to the defined uracil, uridine, or UDP using a searching computer program, or replacing portions of the compound with similar chemical structures from a database using a compound-building computer program.

34. A method for designing potential modulators that are inhibitors of a glycosyltransferase preferably GnT I, GnT V. and/or Core 2L GnT, comprising the step of using one or more (preferably all) of the structural coordinates of UDP-GlcNAc of Tables 17, 18, or 19 as follows:
Table 17 for GnTI transition state
Table 18 for GnTV
Table 19 for core 2 L transition state
to generate a compound for associating with a ligand binding domain of a glycosyltransferase that associates with UDP-GlcNAc.

35. A method for designing potential modulators that are inhibitors of a glycosyltransferase preferably GnT I, GnT V, and/or Core 2L GnT, comprising : (a) generating a computer representation of UDP-GlcNAc defined by the one or more (preferably all) of the structural coordinates of Table 17, 18, or 19 appropriate for a specific glycosyltransferase; (b) searching for molecules in a data base that are structurally or chemically similar to the defined UDP-GlcNAc using a searching computer program, or replacing portions of the compound with similar chemical structures from a database using a compound building computer program.

36. A method for designing potential modulators that are inhibitors of GnT I comprising the step of using one or more (preferably all) of the structural coordinates of Table 20 for an oligosaccharide acceptor, to generate a compound for associating with a ligand binding domain of a glycosyltransferase that associates with the acceptor.

37. A method as claimed in claim 36 comprising: (a) generating a computer representation of an oligosaccharide acceptor defined by the one or more (preferably all) of the structural coordinates of Table 20 appropriate for a specific glycosyltransferase; (b) searching for molecules in a data base that are structurally or chemically similar to the defined oligosaccharide acceptor using a searching computer program, or replacing portions of the compound with similar chemical structures from a database using a compound building computer program.

38. A modulator identified by a method of claim 27, 28, 31, 32, 34, 35, 36, or 37.

39. A modulator of a glycosyltransferase, preferably GnT I, GnT V, and/or Core 2L GnT, comprising the structure of uracil, uridine, ribose, pyrophosphate, or UDP with one or more (preferably all) of the structural coordinates of uracil, uridine, ribose, pyrophosphate, or UDP of Tables 14, 15 or 16 as follows:
Table 14 for GntI Ground State
Table 15 for GnTV
Table 16 for core 2L

40. A modulator of a glycosyltransferase, preferably GnT I, GnT V, and/or Core 2L GnT, comprising the structure of UDP-GlcNAc and having one or more (preferably all) of the structural coordinates of UDP-GlcNAc of Tables 17, 18, or 19 as follows:
Table 17 for GnTI transition state
Table 18 for GnTV,
Table 19 for core 2L

41. A modulator of a glycosyltransferase, preferably GnT I, GnT V, and/or Core 2L, of the Formula I having the structural coordinates of uracil of Table 14, 15 or 16 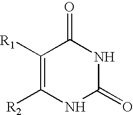
wherein R₁ and R₂ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, pyrophophate, gallic acid, phosphonates, thioamide, and —OR₁₀ where R₁₀ is alkyl, cycloalkyl, alkenyl, alkynyl, or heterocyclic ring;
and salts and optically active and racemic forms of a compound of the formula I.

42. A modulator of a glycosyltransferase, preferably GnT I, GnT V, and/or Core 2L, of the formula II having the structural coordinates of uridine of Table 14, 15, or 16 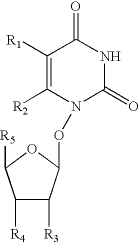
wherein R₁, R₂, R₃, R₄, and R₅ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, pyrophosphate, gallic acid, phosphonates, thioamide, and —OR₁₀ where R₁₀ is alkyl, cycloalkyl, alkenyl, alkynyl, or heterocyclic ring,
and salts and optically active and racemic forms of a compound of the formula II.

43. A modulator of a glycosyltransferase, preferably GnT I, GnT V, and/or Core 2L of the formula III having the structural coordinates of UDP of Tables 14, 15, or 16 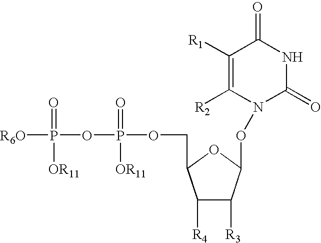
wherein R₁, R₂, R₃, R₄, R₅, and R₆ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate, gallic acid, phosphonates, thioamide, and —OR₁₀ where R₁₀ is alkyl, cycloalkyl, alkenyl, alklynyl, or heterocyclic ring, R₆ may be a monosaccharide or disaccharide, preferably a monosaccharide, including GlcNAc, glucose, and mannose,
and salts and optically active and racemic forms of a compound of the formula III.

44. A modulator of a glycosyltransferase, preferably GnT I, GnT V, and/or Core 2L of the formula IV having the structural coordinates of UDP-GlcNAc of Table 17, 18, or 19 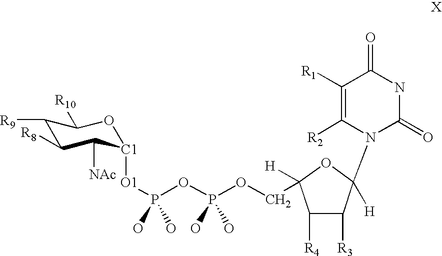
wherein R₁, R₂, R₃, R₄, and R₅ are each independently hydrogen, alkyl, cycloalkyl, alkenyl, alkynyl, heterocyclic rings, aryl, alkoxy, aryloxy, hydroxyl, thiol, thioaryl, amino, halogen, carboxylic acid or esters or thioesters thereof, amines, sulfate, sulfonic or sulfinic acid or esters thereof, phosphate,
gallic acid, phosphonates, thioamide, and —OR₁₀ where R₁₀ is alkyl, cycloalkyl, alkenyl, alkynyl, or heterocyclic ring,
and salts and optically active and racemic forms of a compound of the formula IV.

45. A modulator of a glycosyltransferase comprising the structure of an acceptor of a glycosyltransferase, preferably as shown in FIG. 19A or 33, and the structural coordinates as shown in Table 20.

46. A modulator of a transition state of a glycosyltransferase comprising the structural coordinates of GlcNAc in the transition state of a reaction catalyzed by a glycosyltransferase, preferably Core 2 GnT-L and GnT-I, wherein the GlcNAc has a half chair or distorted chair conformation, a partial double bond between C1 and 05, and a hybridization Sp² at C1.

47. A modulator as claimed in claim 46 wherein the GlcNAc is directly or indirectly linked to a pyrophosphate group and the distance between the pyrophosphate group and the GlcNAc is about 1.9 to 3.5 Å, preferably 2.2 to 3.0 Å.

48. A peptide of the following formula which interferes with the association of the loop structure of a Core 2 transferase and a pyrophosphate group of a sugar nucleotide donor for the Core 2 transferase:
X-X¹-X²-X³-X⁴
wherein X represents 0 to 70, preferably 0 to 50 amino acids, more preferably 2 to 20 amino acids, X¹ and X² independently represent an amino acid with a charged polar group, preferably Glu, Asp, Asn, or Gln, X³ represents a basic amino acid, preferably Arg, His, or Lys, and X⁴ represents 0 to 70, preferably 0 to 50 amino acids, more preferably 2 to 20 amino acids.

49. A pharmaceutical composition comprising a ligand or modulator according to any preceding claim, and optionally a pharmaceutically acceptable carrier, diluent, excipient, or adjuvant or any combination thereof.

50. A method of treating and/or preventing disease comprising the step of administering a ligand or modulator according to any preceding claim or pharmaceutical composition comprising a modulator to a mammalian patient.

51. A method of treating a disease associated with a glycosyltransferase with inappropriate activity in a cellular organism, comprising:
(a) administering a modulator as claimed in any of the preceding claims in an acceptable pharmaceutical preparation; and
(b) activating or inhibiting a glycosyltransferase to treat the disease.

52. Use of a modulator as claimed in any of the preceding claims in the preparation of a medicament to treat a disease associated with a glycosyltransferase with inappropriate activity in a cellular organism.

53. Use of the structural coordinates of a glycosyltransferase as shown in Table 1, 2, 3, 4, 5, 6, 7, or 8 in the manufacture of a medicament.

54. A computer for producing a model or three-dimensional representation of a molecule or molecular complex, wherein said molecule or molecular complex comprises a glycosyltransferase or ligand binding domain thereof defined by structural coordinates of glycosyltransferase amino acids or a ligand binding domain thereof, or comprises structural coordinates of atoms of a ligand or substrate, or a three-dimensional representation of a homologue of said molecule or molecular complex, wherein said computer comprises:
(a) a machine-readable data storage medium comprising a data storage material encoded with machine readable data wherein said data comprises the structural coordinates of glycosyltransferase amino acids according to Table 1, 3, 4, 5, 6, 7, or 8 or a ligand binding domain thereof, or a ligand according to any one of Table 14 through 23;
(b) a working memory for storing instructions for processing said machine-readable data;
(c) a central-processing unit coupled to said working memory and to said machine-readable data storage medium for processing said machine readable data into said three-dimensional representation; and
(d) a display coupled to said central-processing unit for displaying said three-dimensional representation.

55. A method of conducting a drug discovery business comprising:
(a) providing one or more systems or methods for identifying modulators based on a model according to any preceding claim;
(b) conducting therapeutic profiling of modulators identified in step (a), or further analogs thereof, for efficacy and toxicity in animals; and
(c) formulating a pharmaceutical composition including one or more agents identified in step (b) as having an acceptable therapeutic profile.

56. A method as claimed in claim 55 including establishing a distribution system for distributing the pharmaceutical composition for sale, and optionally establishing a sales group for marketing the pharmaceutical composition.

57. A method of conducting a target discovery business comprising:
(a) providing one or more system or method for identifying modulators based on a model as claimed in any preceding claim;
(b) optionally conducting therapeutic profiling of modulators identified in (a) for efficacy and toxicity in animals; and
(c) licensing to a third party the rights for further drug development and/or sales for agents identified in step (a), or analogs thereof.
